(19) United States
(12) Patent Application Publication
(10) Pub. No.: US 2024/0254386 A1
(43) Pub. Date: Aug. 1, 2024

(54) LIGHT EMITTING ELEMENT AND AMINE COMPOUND FOR THE SAME

(71) Applicant: Samsung Display Co., Ltd., Yongin-si (KR)

(72) Inventors: XiuLan JIN, Yokohama (JP); Ichinori TAKADA, Yokohama (JP)

(21) Appl. No.: 18/461,930

(22) Filed: Sep. 6, 2023

(30) Foreign Application Priority Data
Dec. 7, 2022 (KR) 10-2022-0170099

(51) Int. Cl. C09K 11/06; C07D 405/14; C07D 407/14; C07D 409/12; C07D 409/14; H10K 50/13; H10K 85/60

(52) U.S. Cl. CPC C09K 11/06; C07D 405/14; C07D 407/14; C07D 409/12; C07D 409/14; H10K 50/13; H10K 85/6574; H10K 85/6576; C09K 2211/1022

(57) ABSTRACT

A light emitting element includes a first electrode, a second electrode on the first electrode, and at least one functional layer (e.g., hole transport layer) disposed between the first electrode and the second electrode. The functional layer includes a tertiary amine compound represented by Formula 1. The tertiary amine compound is configured so that the hole transport properties of the functional layer (e.g., hole transport layer) and the emission efficiency and lifetime of the light emitting element may be enhanced or improved.

### CROSS-REFERENCE TO RELATED APPLICATIONS

This application claims priority to and the benefit of Korean Patent Application No. 10-2022-0170099, filed on Dec. 7, 2022, the entire content of which is hereby incorporated by reference.

### BACKGROUND

### 1. Field

One or more aspects of embodiments of the present disclosure relate to a light emitting element, for example, a light emitting element with enhanced or improved emission efficiency and lifetime (lifespan), a novel amine compound utilized therein, and a display device including the light emitting element.

### 2. Description of the Related Art

Recently, the development of an organic electroluminescence display device to be utilized as an image display device has been actively conducted. The organic electroluminescence display device includes a “self-luminescent light emitting element” that enables display of images by recombining holes and electrons injected from a first electrode and a second electrode in an emission layer. Subsequently, a light emitting material located in the emission layer emits light to achieve display.

Implementation of the organic electroluminescence device in a display device requires (or there is a desire) that the light emitting element (e.g., self-luminescent light emitting element) possess reduced driving voltage and an improved efficiency and/or long lifetime. Therefore, the need exists for the development of a material for a light emitting element which is capable of stably (or suitably) implementing these properties. For example, in an effort to implement a light emitting element having high luminance emission efficiency and long lifetime, the development of materials for a hole transport region having excellent or suitable hole transport properties and stability is being pursued.

### SUMMARY

One or more aspects of embodiments of the present disclosure is directed toward a light emitting element having enhanced or increased efficiency (e.g., luminance emission efficiency) and/or lifetime, and an amine compound that is included in the light emitting element. Additional aspects will be set forth in part in the description which follows and, in part, will be apparent from the description, or may be learned by practice of the presented embodiments of the disclosure.

An embodiment of the present disclosure relates to a light emitting element that includes a first electrode, a second electrode located or disposed on the first electrode. In one or more embodiments, at least one functional layer may be located or disposed between the first electrode and the second electrode. In one or more embodiments, the at least one functional layer may include an amine compound represented by Formula 1.

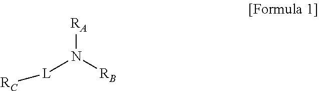

In Formula 1, R_{A} is represented by Formula 2, R_{B} is represented by Formula 3, R_{C} is a substituted or unsubstituted aryl group of 6 to 30 ring-forming carbon atoms, or a substituted or unsubstituted heteroaryl group of 2 to 30 ring-forming carbon atoms, L is a direct linkage, or a substituted or unsubstituted arylene group of 6 to 30 ring-forming carbon atoms. In Formula 2 and Formula 3, in a case where R_{C} is a substituted or unsubstituted benzonaphthofuran group, a substituted or unsubstituted benzonaphthothiophene group, or a substituted or unsubstituted benzofluorene group is excluded, (i.e., R_{C} may not be a substituted or unsubstituted benzonaphthofuran group, a substituted or unsubstituted benzonaphthothiophene group, or a substituted or unsubstituted benzofluorene group). In Formula 2 and Formula 3, in a case where, or when, R_{C} is a substituted or unsubstituted dibenzofuran group, or a substituted or unsubstituted dibenzothiophene group, then L is a direct linkage. In Formula 2 and Formula 3, in a case where, or when. R_{C} is a substituted or unsubstituted carbazole group, then L is a direct linkage, or a substituted or unsubstituted phenylene group.

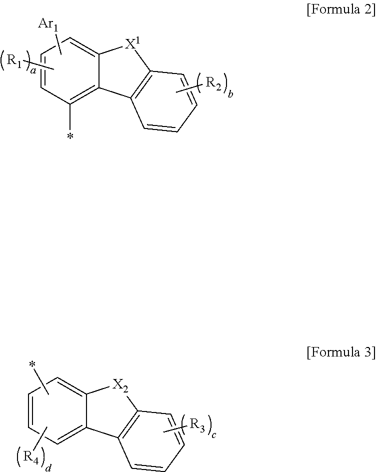

In Formula 2 and Formula 3, Ar₁ is a substituted or unsubstituted aryl group of 6 to 30 ring-forming carbon atoms, X₁ and X₂ may each independently be O or S, and R₁ to R₄ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula 2 and Formula 3, a case where R₁ to R₄ each includes a substituted or unsubstituted nitrogen-containing six-member heterocycle is excluded, (i.e., R₁ to R₄ may not each be a substituted or unsubstituted nitrogen-containing six-member heterocycle), a case where Ar₁ includes a substituted or unsubstituted dihydronaphthyl group, or a substituted or unsubstituted p-aminophenyl group is excluded, (i.e., Ar₁ may not be a substituted or unsubstituted dihydronaphthyl group, or a substituted or unsubstituted p-aminophenyl group), and a case where R₃ includes a substituted or unsubstituted fluorene group is excluded, (i.e., R₃ may not be a substituted or unsubstituted fluorene group). In Formula 2 and Formula 3, a is an integer of 0 to 2, d is an integer of 0 to 3, b and c may each independently be an integer of 0 to 4.

In Formula 2 when X₁ is O, Formula 3 may not be represented by Formula 3-a.

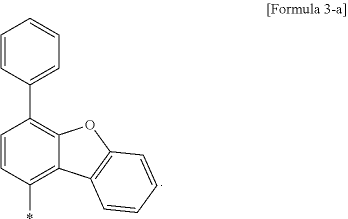 [SMILES: *c1ccc(-c2ccccc2)c2oc3ccccc3c12]

In an embodiment, the at least one functional layer may include an emission layer, a hole transport region located or disposed between the first electrode and the emission layer, and an electron transport region located or disposed between the emission layer and the second electrode. In an embodiment, the hole transport region may include the amine compound represented by Formula 1.

In an embodiment, the hole transport region may include a hole injection layer located or disposed on the first electrode, and a hole transport layer located or disposed on the hole injection layer, and the hole transport layer may include the amine compound represented by Formula 1.

In an embodiment, the hole transport region comprises multiple layers, and a layer of the multiple layers may be adjacent to the emission layer. In an embodiment, the layer of the multiple layers adjacent to the emission layer may include the amine compound represented by Formula 1.

In an embodiment, the amine compound represented by Formula 1 may be a monoamine compound.

In an embodiment, the amine compound represented by Formula 1 may be represented by Formula 1-1.

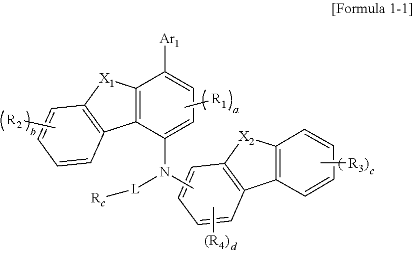

In Formula 1-1, the same explanations defined in Formula 1, Formula 2 and Formula 3 may be applied for R_{C}, L, Ar₁, X₁, X₂, R₁ to R₄, and a to d.

In an embodiment, the amine compound represented by Formula 1 may be represented by Formula 1-2-1 or Formula 1-2-2.

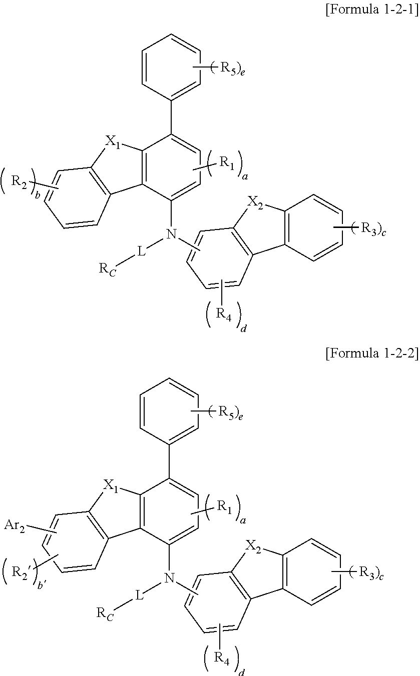

In Formula 1-2-1 and Formula 1-2-2, R₂′ and R₅ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, Ar₂ is a substituted or unsubstituted aryl group of 6 to 30 ring-forming carbon atoms, or a substituted or unsubstituted heteroaryl group of 2 to 30 ring-forming carbon atoms, b′ is an integer of 0 to 3, and e is an integer of 0 to 5.

In Formula 1-2-1 and Formula 1-2-2, the same explanations defined in Formula 1, Formula 2, and Formula 3 may be applied for R_{C}, L, X₁, X₂, R₁ to R₄, and a to d.

In an embodiment, the substituent represented by Formula R_{C} may be represented by any one selected from among Formula A-1 to Formula A-8.

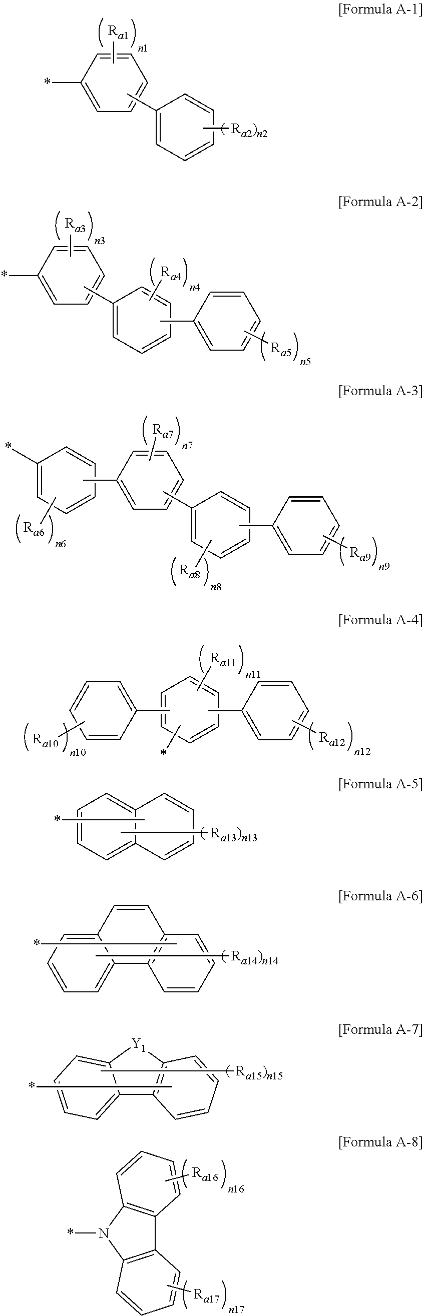

In Formula A-1 to Formula A-8, Y1 may be O, S, CRₐ₁₈Rₐ₁₉, or NRₐ₂₀, and Rₐ₁ to Rₐ₁₉ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula A-1 to Formula A-8, Rₐ₂₀ is a substituted or unsubstituted aryl group of 6 to 30 ring-forming carbon atoms, or a substituted or unsubstituted heteroaryl group of 2 to 30 ring-forming carbon atoms, n1, n3, n4, and n6 to n8 may each independently be an integer of 0 to 4, n2, n5, n9, n10, and n12 may each independently be an integer of 0 to 5, n11 is an integer of 0 to 3, n13 and n15 may each independently be an integer of 0 to 7, n14 is an integer of 0 to 9, n16 and n17 may each independently be an integer of 0 to 4, and -* is a position connected with L of Formula 1.

In an embodiment, the amine compound represented by Formula 1 may be represented by any one selected from among Formula 1-4-1 to Formula 1-4-3.

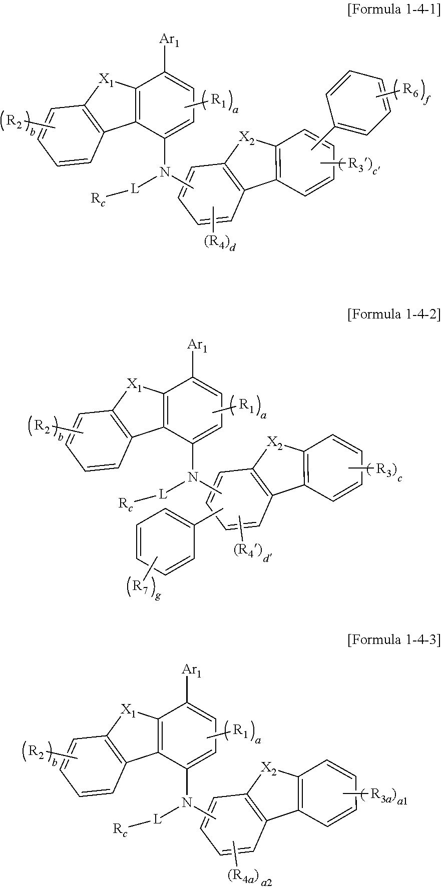

In Formula 1-4-1 to Formula 1-4-3, R₃′, R₄′, R₆ and R₇ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, R₃ₐ and R₄ₐ may each independently be a hydrogen atom, or a deuterium atom, f and g may each independently be an integer of 0 to 5, c′ and a2 may each independently be an integer of 0 to 3, d′ is an integer of 0 to 2, and a1 is an integer of 0 to 4.

In Formula 1-4-1 to Formula 1-4-3, the same explanations defined in Formula 1, Formula 2, and Formula 3 may be applied for R_{C}, L, Ar₁, X₁, X₂, R₁ to R₄, and a to d.

In an embodiment, the amine compound represented by Formula 1 may be represented by any one selected from among Formula 1-5-1 or Formula 1-5-2.

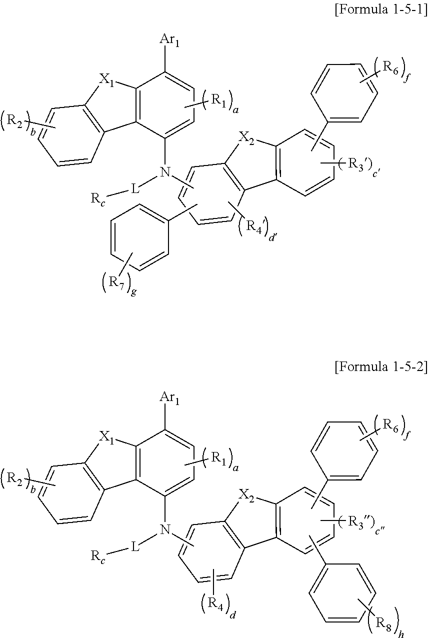

In Formula 1-5-1 and Formula 1-5-2, R₃′, R₃″, R₄′, and R₆ to R₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, f to h may each independently be an integer of 0 to 5, c′ is an integer of 0 to 3, and c″ and d′ may each independently be an integer of 0 to 2.

In Formula 1-5-1 and Formula 1-5-2, the same explanations defined in Formula 1, Formula 2, and Formula 3 may be applied for R_{C}, L, Ar₁, X₁, X₂, R₁ R₂, R₄, a, b, and d.

In an embodiment, the amine compound represented by Formula 1 may be represented by any one selected from among Formula 1-6-1 to Formula 1-6-4.

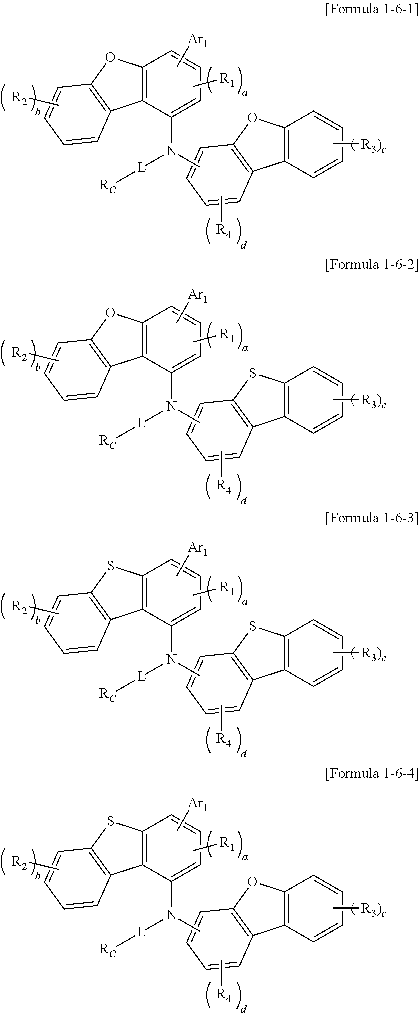

In Formula 1-6-1 to Formula 1-6-4, the same explanations defined in Formula 1, Formula 2, and Formula 3 may be applied for R_{C}, L, Ar₁, R₁ to R₄, and a to d.

In an embodiment, the amine compound represented by Formula 1 may be represented by any one selected from among Formula 1-7-1 or Formula 1-7-2.

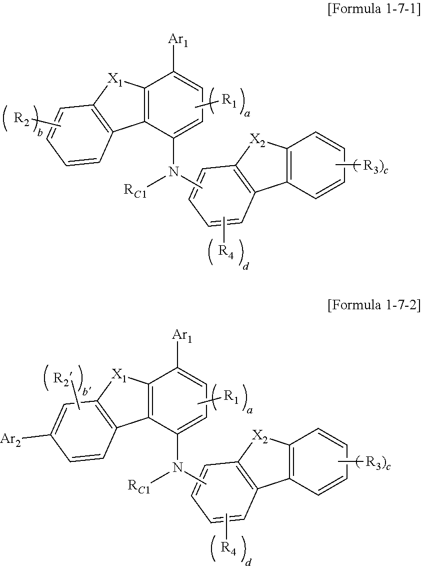

In Formula 1-7-1 and Formula 1-7-2, R₂′ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, Ar₂ is a substituted or unsubstituted aryl group of 6 to 30 ring-forming carbon atoms, or a substituted or unsubstituted heteroaryl group of 2 to 30 ring-forming carbon atoms, b′ is an integer of 0 to 3, and R_{C1} is selected from Compound Group A.

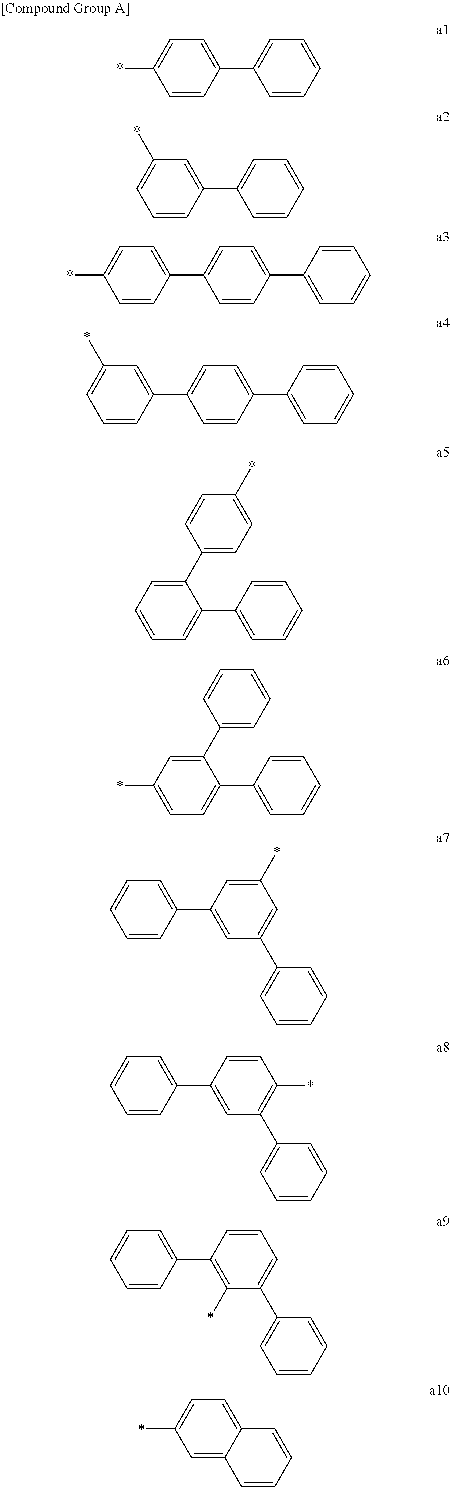

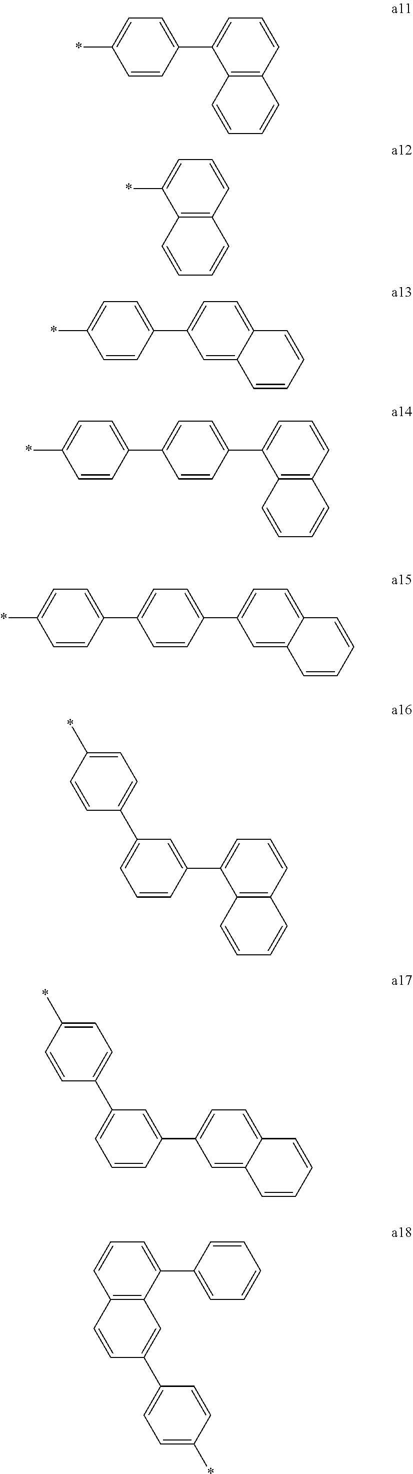

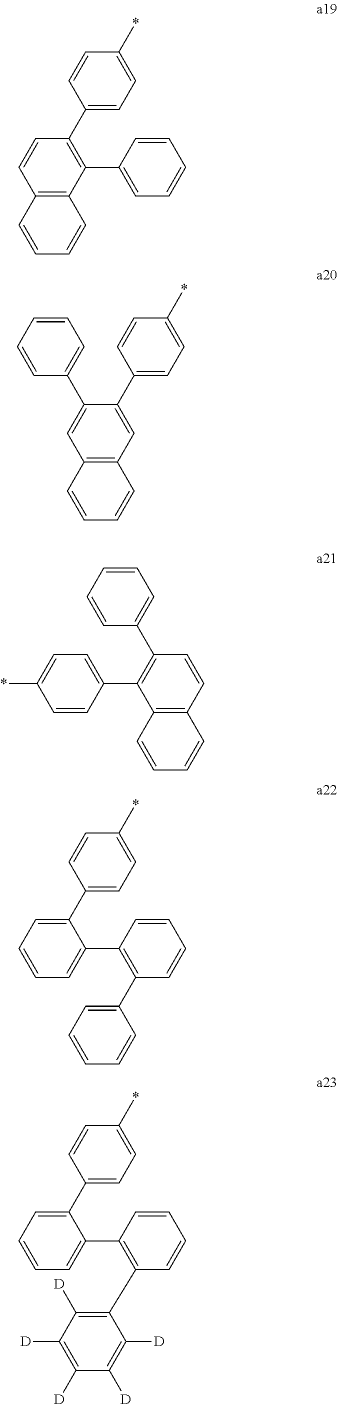

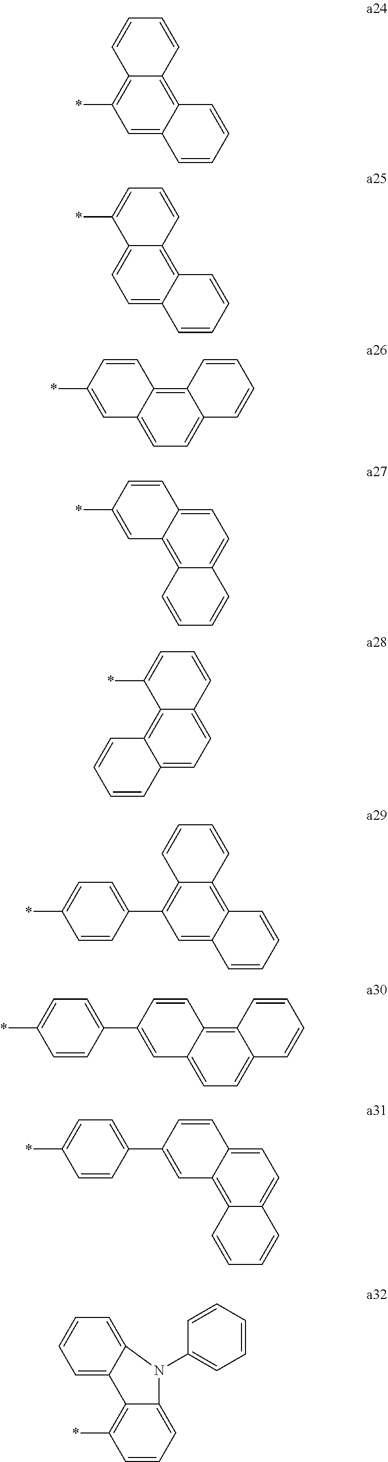

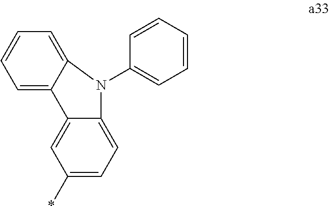 [SMILES: *c1ccc2c(c1)c1ccccc1n2-c1ccccc1]

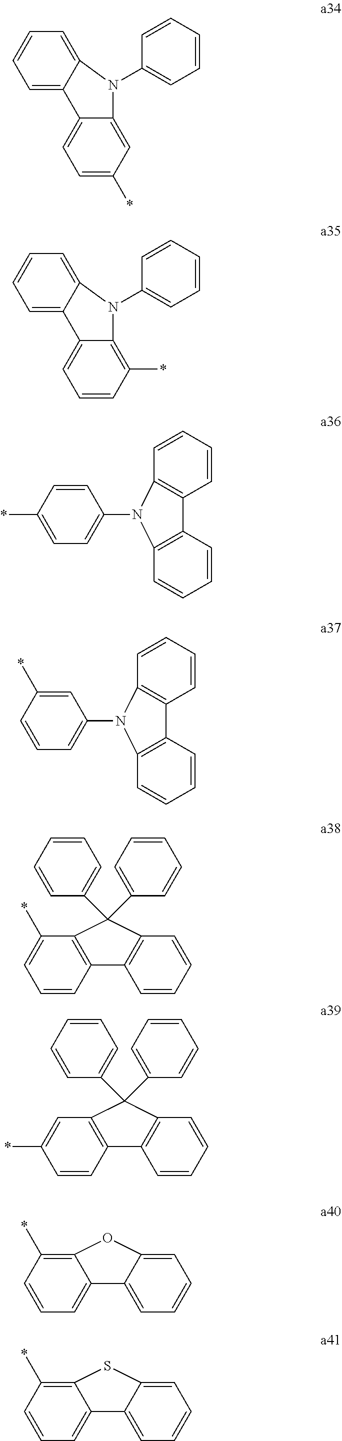

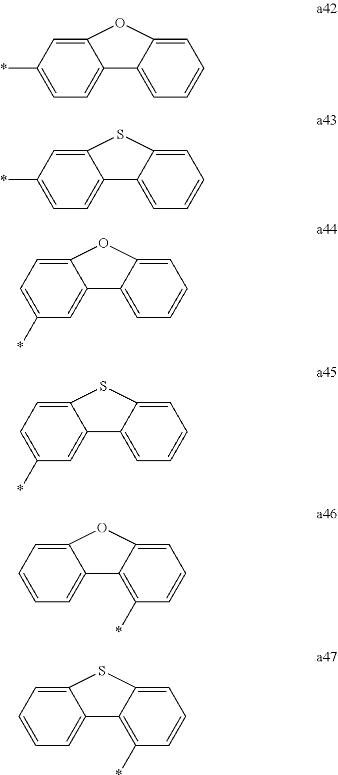

In Formula 1-7-1 and Formula 1-7-2, the same explanations defined in Formula 1, Formula 2, and Formula 3 may be applied for Ar₁, X₁, X₂, R₁ to R₄, and a to d.

In an embodiment, L may be represented by any one selected from among Formula L-1 to Formula L-6.

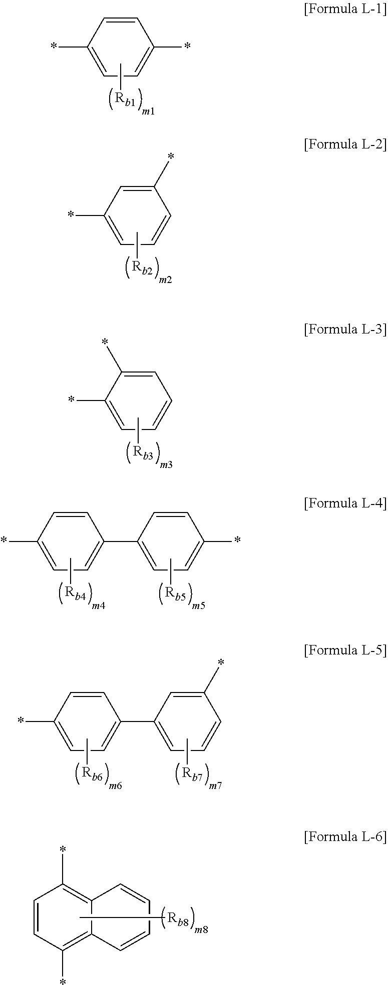

In Formula L-1 to Formula L-6, R_{b1} to R_{b8} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, m1 to m7 may each independently be an integer of 0 to 4, and m8 may be an integer of 0 to 6.

An amine compound according to an embodiment of the present disclosure is represented by Formula 1.

### BRIEF DESCRIPTION OF THE DRAWINGS

The accompanying drawings are included to provide a further understanding of the present disclosure and are incorporated in and constitute a part of this specification. The drawings illustrate example embodiments of the present disclosure and, together with the description, serve to explain principles of the present disclosure. In the drawings:

FIG. 1 is a plan view of a display device according to one or more embodiments of the present disclosure;

FIG. 2 is a cross-sectional view of a display device according to one or more embodiments of the present disclosure;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure;

FIG. 10 is a cross-sectional view showing a display device according to one or more embodiments of the present disclosure; and

FIG. 11 is a diagram showing a vehicle including display apparatus according to one or more embodiments of the present disclosure.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure. Unless otherwise defined, all chemical names, technical and scientific terms, and terms defined in common dictionaries should be interpreted as having meanings consistent with the context of the related art, and should not be interpreted in an ideal or overly formal sense.

Like reference numerals refer to like elements throughout, and duplicative descriptions thereof may not be provided. In the drawings, the dimensions of structures are exaggerated for clarity of illustration. It will be understood that, although the terms first, second, etc. may be utilized herein to describe one or more suitable elements, these elements should not be limited by these terms. These terms are only utilized to distinguish one element from another element. Thus, a first element could be termed a second element without departing from the teachings of the present disclosure. Similarly, a second element could be termed a first element. As utilized herein, the singular forms are intended to include the plural forms as well, unless the context clearly indicates otherwise.

In the application, it will be further understood that the terms “comprise,” “comprises,” “comprising,” “has,” “have,” “having,” “include,” “includes,” and/or “including,” when utilized in this specification, specify the presence of stated features, numerals, steps, operations, elements, parts, or the combination thereof, but do not preclude the presence or addition of one or more other features, numerals, steps, operations, elements, parts, or the combination thereof.

As used herein, the terms “use,” “using,” and “used” may be considered synonymous with the terms “utilize,” “utilizing,” and “utilized,” respectively.

As used herein, expressions such as “at least one of,” “one of,” and “selected from,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

As used herein, the term “and/or” includes any and all combinations of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “coupled to” another element, it may be directly on, connected, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

Unless otherwise defined, all terms (including technical and scientific terms) used herein have the same meaning as commonly understood by those of ordinary skill in the art to which the present disclosure pertains. It is also to be understood that terms defined in commonly used dictionaries should be interpreted as having meanings consistent with meanings in the context of the related art, unless expressly defined herein, and should not be interpreted in an ideal or overly formal sense.

In this context, “consisting essentially of” means that any additional components will not materially affect the chemical, physical, optical, or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

### Definitions

In the description, the term “substituted or unsubstituted” corresponds to substituted or unsubstituted with at least one substituent selected from the group consisting of a deuterium atom, a halogen atom, a cyano group, a nitro group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, an alkoxy group, a hydrocarbon ring group, an aryl group, and a heterocyclic group. In some embodiments, each of the exemplified substituents may be substituted or unsubstituted. For example, a biphenyl group may be interpreted as an aryl group or a phenyl group substituted with a phenyl group.

In the description, the term “forming a ring via the combination with an adjacent group” may refer to forming a substituted or unsubstituted hydrocarbon ring, or a substituted or unsubstituted heterocycle via the combination with an adjacent group. The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In some embodiments, the ring formed via the combination with an adjacent group may be combined with another ring to form a spiro structure.

In the description, the term “adjacent group” may refer to a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In some embodiments, in 4,5-dimethylphenanthrene, two methyl groups may be interpreted as “adjacent groups” to each other.

In the description, a halogen atom may be a fluorine atom, a chlorine atom, a bromine atom or an iodine atom.

In the description, an alkyl group may be a linear, branched, or cyclic type or kind. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, a cycloalkyl group may refer to a ring-type or kind alkyl group. The carbon number of the cycloalkyl group may be 3 to 50, 3 to 30, 3 to 20, or 3 to 10. Examples of the cycloalkyl group include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group etc., without limitation.

In the description, an alkenyl group refers to a hydrocarbon group including one or more carbon double bonds in the middle or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, an allyl group, a 1-butenyl group, a 1-pentenyl group, a 1,3-butadienyl aryl group, a styrenyl group, a styrylvinyl group, etc., without limitation.

In the description, an aryl group refers to an optional functional group or substituent derived from an aromatic hydrocarbon ring. The aryl group may be a monocyclic aryl group or a polycyclic aryl group. The carbon number for forming rings in the aryl group may be 6 to 40, 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysene, etc., without limitation.

In the description, a fluorenyl group may be substituted, and two substituents may be combined with each other to form a spiro structure. Examples of a substituted fluorenyl group are as follows, but an embodiment of the present disclosure is not limited thereto.

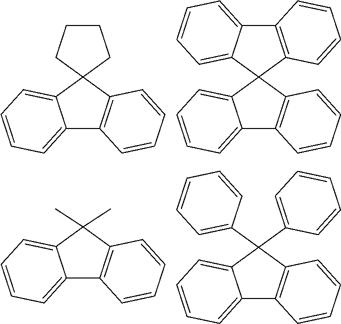

In the description, a heterocyclic group refers to an optional functional group or substituent derived from a ring including one or more among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, a heteroaryl group may include one or more among B, O, N, P, Si, and S as heteroatoms. When the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 40, 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation on the above-described aryl group may be applied to an arylene group except that the arylene group is a divalent group. The same explanation on the above-described heteroaryl group may be applied to a heteroarylene group except that the heteroarylene group is a divalent group.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, a thio group may include an alkyl thio group and an aryl thio group. The thio group may refer to the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may refer to the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, an embodiment of the present disclosure is not limited thereto.

In the description, a boron group may refer to the above-defined alkyl group or aryl group combined with a boron atom. The boron group includes an alkyl boron group and an aryl boron group. Examples of the boron group include a dimethylboron group, a diethylboron group, a t-butylboron group, a diphenylboron group, a diphenylboron group, a phenylboron group, and/or the like, without limitation.

In the description, the carbon number of an amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation.

In the description, a direct linkage may refer to a single bond.

In some embodiments, in the description,

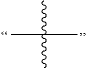

and “-•” refer to positions to be connected.

Hereinafter, embodiments of the present disclosure will be explained referring to the drawings.

### Display Device

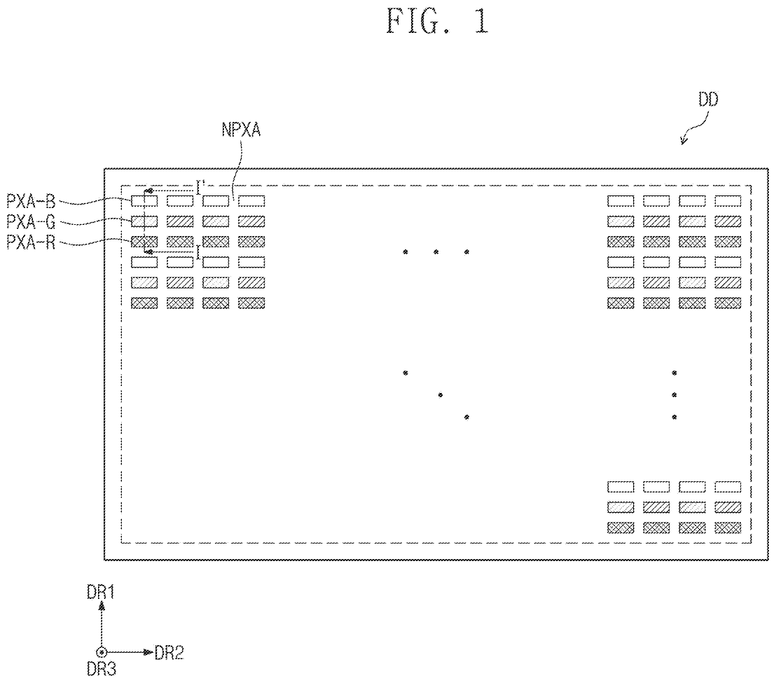

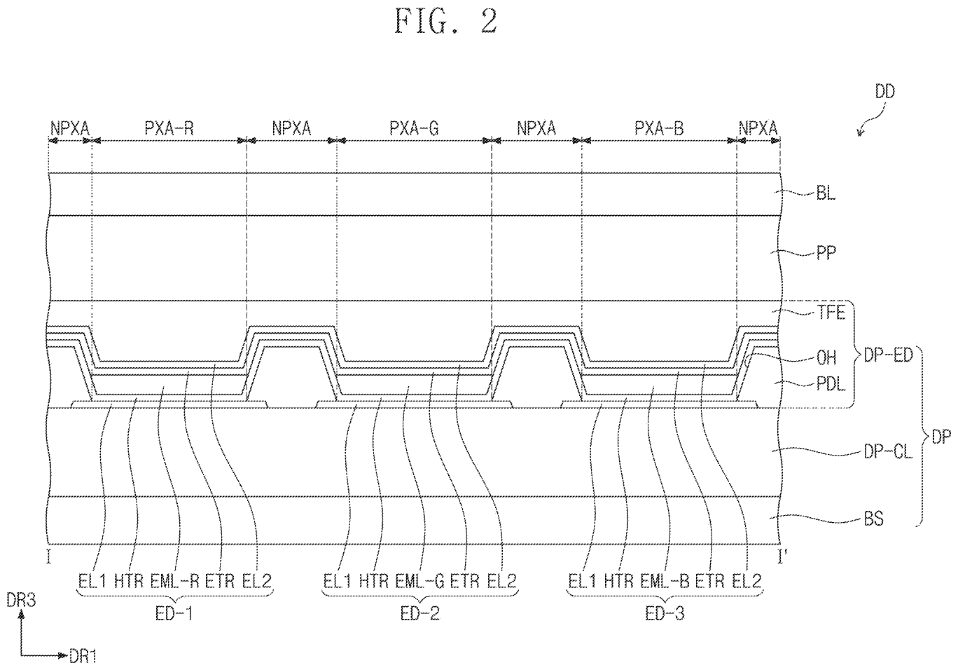

FIG. 1 is a plan view showing an embodiment of a display device DD. FIG. 2 is a cross-sectional view of a display device DD of an embodiment showing a part corresponding to line I-I′ of FIG. 1.

The display device DD may include a display panel DP and an optical layer PP located or disposed on the display panel DP. The display panel DP may include light emitting elements ED-1, ED-2 and ED-3. The display device DD may include multiple light emitting elements ED-1, ED-2 and ED-3. The optical layer PP may be located or disposed on the display panel DP and control reflected light by external light at the display panel DP. The optical layer PP may include, for example, a polarization layer or a color filter layer. In some embodiments, different from the drawings, the optical layer PP may not be included in the display device DD of an embodiment.

On the optical layer PP, a base substrate BL may be located or disposed. The base substrate BL may be a member providing a base surface where the optical layer PP is located or disposed. The base substrate BL may be a glass substrate, a metal substrate, a plastic substrate, and/or the like. However, an embodiment of the present disclosure is not limited thereto, and the base substrate BL may be an inorganic layer, an organic layer and/or a composite material layer. In some embodiments, different from the drawings, the base substrate BL may not be included in an embodiment.

The display device DD according to an embodiment may further include a plugging layer. The plugging layer may be located or disposed between a display element layer DP-ED and a base substrate BL. The plugging layer may be an organic layer. The plugging layer may include at least one among an acrylic resin, a silicon-based resin and an epoxy-based resin.

The display panel DP may include a base layer BS, a circuit layer DP-CL located or provided on the base layer BS and a display element layer DP-ED. The display element layer DP-ED may include a pixel definition layer PDL, light emitting elements ED-1, ED-2 and ED-3 located or disposed in the pixel definition layer PDL, and an encapsulating layer TFE located or disposed on the light emitting elements ED-1, ED-2 and ED-3.

The base layer BS may be a member providing a base surface where the display element layer DP-ED is located or disposed. The base layer BS may be a glass substrate, a metal substrate, a plastic substrate, and/or the like. However, an embodiment of the present disclosure is not limited thereto, and the base layer BS may be an inorganic layer, an organic layer and/or a composite material layer.

In an embodiment, the circuit layer DP-CL is located or disposed on the base layer BS, and the circuit layer DP-CL may include multiple transistors. Each of the transistors may include a control electrode, an input electrode, and an output electrode. For example, the circuit layer DP-CL may include switching transistors and driving transistors for driving the light emitting elements ED-1, ED-2 and ED-3 of the display element layer DP-ED.

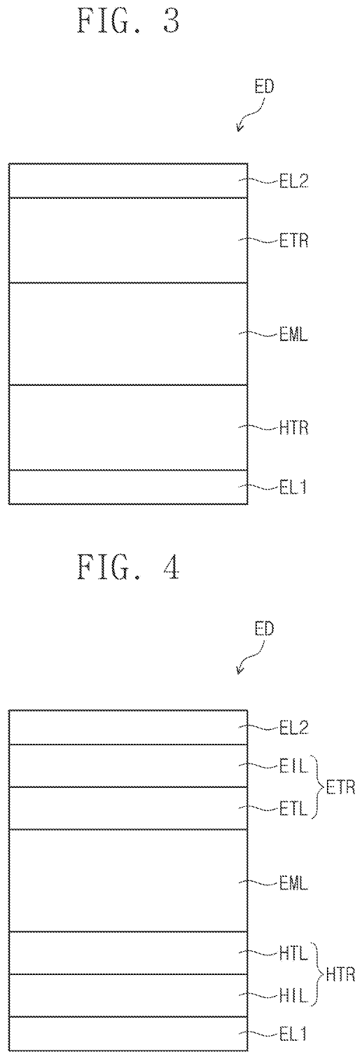

The light emitting elements ED-1, ED-2 and ED-3 may have the structures of the light emitting elements ED of embodiments according to FIG. 3 to FIG. 6, as described in more detail elsewhere herein. The light emitting elements ED-1, ED-2 and ED-3 may include a first electrode EL1, a hole transport region HTR, emission layers EML-R, EML-G and EML-B, an electron transport region ETR and a second electrode EL2.

In FIG. 2, shown is an embodiment where the emission layers EML-R, EML-G and EML-B of light emitting elements ED-1, ED-2 and ED-3 are located or disposed in opening portions OH defined in a pixel definition layer PDL, and a hole transport region HTR, an electron transport region ETR and a second electrode EL2 are provided as common layers in all light emitting elements ED-1, ED-2 and ED-3. However, an embodiment of the present disclosure is not limited thereto. Different from FIG. 2, in an embodiment, the hole transport region HTR and the electron transport region ETR may be patterned or formed and provided in the opening portions OH defined in the pixel definition layer PDL. For example, in an embodiment, the hole transport region HTR, the emission layers EML-R, EML-G and EML-B, and the electron transport region ETR of the light emitting elements ED-1, ED-2 and ED-3 may be patterned or formed by an ink jet printing method and provided.

An encapsulating layer TFE may cover the light emitting elements ED-1, ED-2 and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, encapsulating inorganic layer). In some embodiments, the encapsulating layer TFE according to an embodiment may include at least one organic layer (hereinafter, encapsulating organic layer) and at least one encapsulating inorganic layer.

The encapsulating inorganic layer protects the display element layer DP-ED from moisture and/or oxygen, and the encapsulating organic layer protects the display element layer DP-ED from foreign materials such as dust particles. The encapsulating inorganic layer may include silicon nitride, silicon oxy nitride, silicon oxide, titanium oxide, aluminum oxide, and/or the like. The encapsulating organic layer may include an acrylic compound, an epoxy-based compound, and/or the like. The encapsulating organic layer may include a photopolymerizable organic material, and/or the like.

The encapsulating layer TFE may be located or disposed on the second electrode EL2 and may be located or disposed while filling the opening portion OH.

Referring to FIG. 1 and FIG. 2, the display device DD may include a non-luminous area NPXA and luminous areas PXA-R, PXA-G and PXA-B. The luminous areas PXA-R, PXA-G and PXA-B may be areas emitting light produced from the light emitting elements ED-1, ED-2 and ED-3, respectively. The luminous areas PXA-R, PXA-G and PXA-B may be separated from each other, e.g., when viewed on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. In some embodiments, in the disclosure, each of the luminous areas PXA-R, PXA-G and PXA-B may correspond to each pixel. The pixel definition layer PDL may divide the light emitting elements ED-1, ED-2 and ED-3. The emission layers EML-R, EML-G and EML-B of the light emitting elements ED-1, ED-2 and ED-3 may be located or disposed and divided in the opening portions OH defined in the pixel definition layer PDL.

The luminous areas PXA-R, PXA-G and PXA-B may be divided into multiple groups according to the color of light produced from the light emitting elements ED-1, ED-2 and ED-3. In the display device DD of an embodiment, shown in FIG. 1 and FIG. 2, three luminous areas PXA-R, PXA-G and PXA-B emitting red light, green light and blue light are illustrated as an embodiment. For example, the display device DD of an embodiment may include a red luminous area PXA-R, a green luminous area PXA-G and a blue luminous area PXA-B, which are separated from each other.

In the display device DD according to an embodiment, multiple light emitting elements ED-1, ED-2 and ED-3 may be configured to emit light having different wavelength regions. For example, in an embodiment, the display device DD may include a first light emitting element ED-1 emitting red light, a second light emitting element ED-2 emitting green light, and a third light emitting element ED-3 emitting blue light. For example, each of the red luminous area PXA-R, the green luminous area PXA-G, and the blue luminous area PXA-B of the display device DD may correspond to the first light emitting element ED-1, the second light emitting element ED-2, and the third light emitting element ED-3, respectively.

However, an embodiment of the present disclosure is not limited thereto, and the first to third light emitting elements ED-1, ED-2 and ED-3 may be configured to emit light in substantially the same wavelength region, or at least one thereof may be configured to emit light in a different wavelength region. For example, all the first to third light emitting elements ED-1, ED-2 and ED-3 may be configured to emit blue light.

The luminous areas PXA-R, PXA-G and PXA-B in the display device DD according to an embodiment may be arranged in a stripe shape. Referring to FIG. 1, multiple red luminous areas PXA-R may be arranged with each other along a second directional axis DR2, multiple green luminous areas PXA-G may be arranged with each other along the second directional axis DR2 and multiple blue luminous areas PXA-B may be arranged with each other along the second directional axis DR2. In some embodiments, the (one) red luminous area PXA-R, the (one) green luminous area PXA-G and the (one) blue luminous area PXA-B may be arranged with each other by turns along a first directional axis DR1.

In FIG. 1 and FIG. 2, the areas of the luminous areas PXA-R, PXA-G and PXA-B are shown similar, but an embodiment of the present disclosure is not limited thereto. The areas of the luminous areas PXA-R, PXA-G and PXA-B may be different from each other according to the wavelength region of light emitted. In some embodiments, the areas of the luminous areas PXA-R, PXA-G and PXA-B may refer to areas on a plane defined by the first directional axis DR1 and the second directional axis DR2 (or areas in a plan view).

In some embodiments, the arrangement type or kind of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in one or more suitable combinations according to the properties of display quality required for the display device DD. For example, the arrangement type or kind of the luminous areas PXA-R, PXA-G and PXA-B may be the PENTILE® arrangement form or the Diamond Pixel™ arrangement form (PENTILE® is a registered trademark owned by Samsung Display Co., Ltd.).

In some embodiments, the size(s) or area(s) of the luminous areas PXA-R, PXA-G and PXA-B may be different from each other. For example, in an embodiment, the area of the green luminous area PXA-G may be smaller than the area of the blue luminous area PXA-B, but an embodiment of the present disclosure is not limited thereto.

### Light Emitting Element

FIG. 3 to FIG. 6 are each a cross-sectional view schematically showing light emitting element according to an embodiment of the present disclosure. The light emitting element ED according to an embodiment may include a first electrode EL1, a second electrode EL2 oppositely located or disposed to the first electrode EL1, and at least one functional layer located or disposed between the first electrode EL1 and the second electrode EL2. The light emitting element ED of an embodiment may include an amine compound of an embodiment, as described elsewhere herein, in the at least one functional layer.

The light emitting element ED may include a hole transport region HTR, an emission layer EML, an electron transport region ETR, and/or the like, stacked in order, as the at least one functional layer.

Referring to FIG. 3, the light emitting element ED of an embodiment may include a first electrode EL1, a hole transport region HTR, an emission layer EML, an electron transport region ETR, and a second electrode EL2, stacked in order (e.g., sequentially laminated).

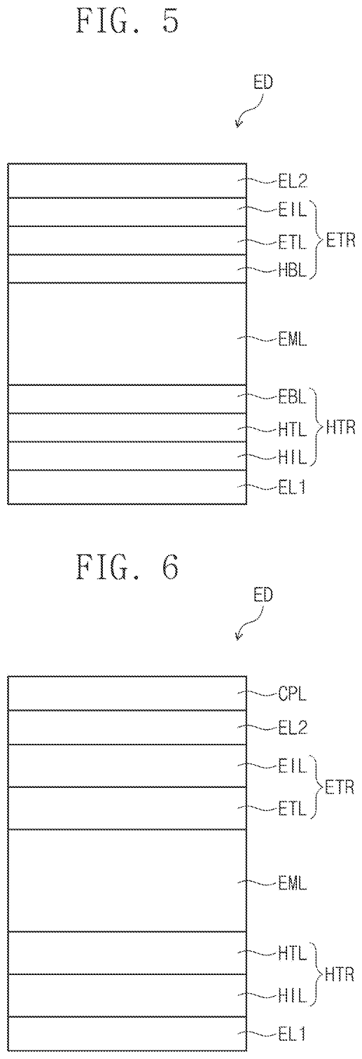

When compared to FIG. 3, FIG. 4 shows the cross-sectional view of a light emitting element ED of an embodiment, wherein a hole transport region HTR includes a hole injection layer HIL and a hole transport layer HTL, and an electron transport region ETR includes an electron injection layer EIL and an electron transport layer ETL. In some embodiments, when compared to FIG. 3, FIG. 5 shows the cross-sectional view of a light emitting element ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared to FIG. 4, FIG. 6 shows the cross-sectional view of a light emitting element ED of an embodiment, including a capping layer CPL located or disposed on the second electrode EL2.

The light emitting element ED of an embodiment may include an amine compound of an embodiment, as described elsewhere herein, in a hole transport region HTR. The light emitting element ED of an embodiment may include an amine compound of an embodiment in at least one among the hole injection layer HIL, hole transport layer HTL and electron blocking layer EBL of the hole transport region HTR. For example, in the light emitting element ED of an embodiment, the hole transport layer HTL may include the amine compound of an embodiment.

In the light emitting element ED according to an embodiment, the first electrode EL1 has conductivity (e.g., is a conductor). The first electrode EL1 may be formed utilizing a metal material, a metal alloy or a conductive compound. The first electrode EL1 may be an anode or a cathode. However, an embodiment of the present disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected among Ag, Mg, Cu, Al, Pt, Pd, Au, Ni, Nd, Ir, Cr, Li, Ca, LiF, Mo, Ti, W, In, Sn, Zn, compounds of two or more selected therefrom, mixtures of two or more selected therefrom, and/or oxides thereof.

When the first electrode EL1 is the transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compound(s) thereof, or mixture(s) thereof (for example, a mixture of Ag and Mg). In some embodiments, the first electrode EL1 may have a structure including multiple layers including a reflective layer or a transflective layer formed utilizing the above materials, and a transmissive conductive layer formed utilizing ITO, IZO, ZnO, and/or ITZO. For example, the first electrode EL1 may have a three-layer structure of ITO/Ag/ITO. However, an embodiment of the present disclosure is not limited thereto. The first electrode EL1 may include the described metal materials, combinations of two or more metal materials selected from the described metal materials, or oxides of the described metal materials. The thickness of the first electrode EL1 may be from about 700 angstrom (Å) to about 10,000 Å. For example, the thickness of the first electrode EL1 may be from about 1,000 Å to about 3,000 Å.

The hole transport region HTR is located or provided on the first electrode EL1. The hole transport region HTR may have a single layer formed utilizing a single material, a single layer formed utilizing multiple different materials, or a multilayer structure including multiple layers formed utilizing multiple different materials.

The hole transport region HTR may include at least one of a hole injection layer HIL, a hole transport layer HTL, or an electron blocking layer EBL. In some embodiments, though not shown, the hole transport region HTR may include multiple hole transport layers that are stacked.

In some embodiments, the hole transport region HTR may have the structure of a single layer of a hole injection layer HIL or a hole transport layer HTL, and may have a structure of a single layer formed utilizing a hole injection material and a hole transport material. In an embodiment, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, or hole transport layer HTL/buffer layer.

The thickness of the hole transport region HTR may be, for example, about 50 Å to about 15,000 Å. The hole transport region HTR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The light emitting element ED of an embodiment may include the amine compound of an embodiment in a hole transport region HTR. In the light emitting element ED of an embodiment, the hole transport region HTR may include an hole injection layer HIL, and a hole transport layer HTL, and the hole transport layer HTL may include the amine compound of an embodiment. The amine compound of an embodiment may be included in a layer adjacent to an emission layer EML among the layers included in the hole transport region HTR.

### Amine Compound

The amine compound of an embodiment includes a structure in which a first substituent, a second substituent and a third substituent are connected to or with a core nitrogen atom. The amine compound of an embodiment includes an amine group, i.e., a core nitrogen atom, and the first substituent, the second substituent, and the third substituent may be connected to or combined with the core nitrogen atom of the amine compound of an embodiment. In an embodiment, each of the first substituent and the second substituent includes (e.g., essentially includes) a dibenzoheterole moiety. In the description, the term “dibenzoheterole” moiety refers to a dibenzofuran moiety or a dibenzothiophene moiety.

The first substituent has or makes a direct linkage to or with the core nitrogen atom at a carbon position 1 (i.e., the carbon atom at position 1) of the first substituent. The first substituent includes a first aryl group connected to (i.e., substituted in) at least one among carbon atoms at positions 2 to 4 of the first substituent, and excluding position 1. The second substituent has or makes a direct linkage to or with the core nitrogen atom. The third substituent may be an aryl group or a heteroaryl group connected to or with the core nitrogen atom via an arylene linker, or directly connected to or with the core nitrogen atom without a separate (e.g., arylene) linker.

In the description, the carbon position numbers of the first substituent is provided as designated in Formula S1.

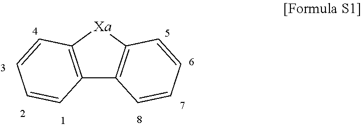

On the provision of the carbon atom position numbers of the first substituent, positioning the first substituent so that Xₐ is positioned at the upper part of Formula S1, the position numbering is provided clockwise from the carbon atom at position 1, (i.e., positioned at the lower part among carbon atoms constituting a left benzene ring), which is the meta position with respect to Xₐ, and the position numbers of carbon atoms at fused positions are excluded. For convenience of explanation, substituents connected with both (e.g., simultaneously) of the benzene rings in Formula S1 are omitted. In an embodiment different from Formula S1 as shown, the first substituent may have at least one substituent other than hydrogen atoms. In an embodiment, the first aryl group may be connected to (i.e., substituted in) at least one among carbon atoms at positions 2 to 4 of the first substituent represented by Formula S1.

In Formula S1, Xₐ is O or S. In Formula S1, when Xₐ is O, the first substituent may include a dibenzofuran moiety. In Formula S1, when Xₐ is S, the first substituent may include a dibenzothiophene moiety.

The amine compound of an embodiment may be a monoamine compound including a single amine group. The amine compound of an embodiment may be a monoamine compound in which only one amine group is present in a state of not forming a ring in a molecular structure.

In an embodiment, the amine compound may be represented by Formula 1.

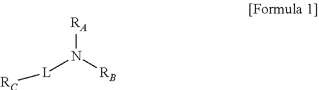

In Formula 1, R_{C} is a substituted or unsubstituted aryl group of 6 to 30 ring-forming carbon atoms, or a substituted or unsubstituted heteroaryl group of 2 to 30 ring-forming carbon atoms. For example, R_{C} may be a substituted or unsubstituted carbazole group, a substituted or unsubstituted phenanthrene group, a substituted or unsubstituted phenanthrylphenyl group, a substituted or unsubstituted biphenyl group, a substituted or unsubstituted terphenyl group, a substituted or unsubstituted quaterphenyl group, a substituted or unsubstituted naphthyl group, a substituted or unsubstituted naphthylphenyl group, a substituted or unsubstituted phenylnaphthyl group, a substituted or unsubstituted dibenzofuran group, a substituted or unsubstituted dibenzothiophene group, or a substituted or unsubstituted fluorene group.

In the amine compound of an embodiment, represented by Formula 1, a case where R_{C} is a substituted or unsubstituted benzonaphthofuran group, a substituted or unsubstituted benzonaphthothiophene group, or a substituted or unsubstituted benzofluorene group is excluded. In other words, R_{C} may not be a substituted or unsubstituted benzonaphthofuran group, a substituted or unsubstituted benzonaphthothiophene group, or a substituted or unsubstituted benzofluorene group.

In Formula 1, L is a direct linkage, or a substituted or unsubstituted arylene group of 6 to 30 ring-forming carbon atoms. For example, L may be a direct linkage, a substituted or unsubstituted phenylene group, a substituted or unsubstituted biphenylene group, or a substituted or unsubstituted naphthylene group.

In the amine compound of an embodiment, represented by Formula 1, in a case where, or when, R_{C} is a substituted or unsubstituted dibenzofuran group, or a substituted or unsubstituted dibenzothiophene group, L is a direct linkage. In some embodiments, in a case where, or when, R_{C} is a substituted or unsubstituted carbazole group, L is a direct linkage, or a substituted or unsubstituted phenylene group.

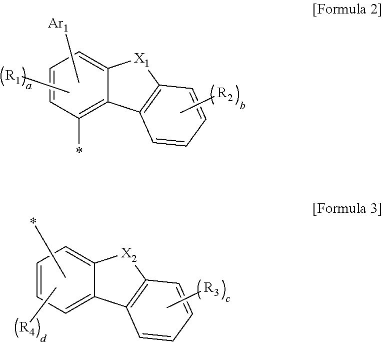

In some embodiments, in the description, the substituent represented by Formula 2 may be a substituent corresponding to the first substituent. In some embodiments, the substituent represented by Formula 3 may be a substituent corresponding to the second substituent.

In Formula 2, Ar₁ is a substituted or unsubstituted aryl group of 6 to 30 ring-forming carbon atoms. In some embodiments, in the amine compound of an embodiment, in a case where Ar₁ is a substituted or unsubstituted dihydronaphthyl group, or a substituted or unsubstituted p-aminophenyl group, is excluded. In other words, Ar₁ may not be a substituted or unsubstituted dihydronaphthyl group, or a substituted or unsubstituted p-aminophenyl group. For example, Ar₁ may be a substituted or unsubstituted phenyl group. In some embodiments, the substituent represented by Ar₁ in Formula 2 may be a substituent corresponding to the first aryl group as described elsewhere herein.

In the description, the term “dihydronaphthyl group” refers to a 1,4-dihydronaphthyl group having a structure of S3 and/or a 1,2-dihydronaphthyl group having a structure of S4.

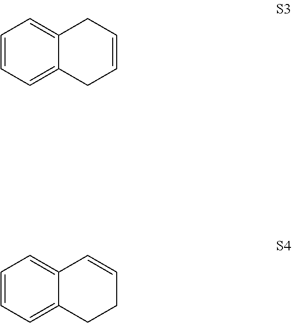 [SMILES: C1=CCc2ccccc2C1 ; C1=Cc2ccccc2CC1]

In the description, the term “p-aminophenyl group” refers to a phenyl group substituted with a substituted or unsubstituted amine group.

In Formula 2 and Formula 3, X₁ and X₂ may each independently be O or S. X₁ and X₂ may be the same or different. In an embodiment, both (e.g., simultaneously) X₁ and X₂ may be O or S. In some embodiments, one among X₁ and X₂ may be O, and the remaining one may be S.

In Formula 2 and Formula 3, R₁ to R₄ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. In some embodiments, in the amine compound of an embodiment, a case where R₃ includes a substituted or unsubstituted fluorene group, is excluded. In other words, R₃ may not be a substituted or unsubstituted fluorene group. In some embodiments, in the amine compound of an embodiment, a case where each of R₁ to R₄ includes a substituted or unsubstituted nitrogen-containing six-member heterocycle, is excluded. In other words, R₁ to R₄ may not each be a substituted or unsubstituted nitrogen-containing six-member heterocycle. For example, in the amine compound of an embodiment, a case where each of R₁ to R₄ is a six-member heterocycle containing at least one or more N as ring-forming atoms is excluded. In other words, R₁ to R₄ may not each be a six-member heterocycle containing at least one or more N as ring-forming atoms. For example, in the amine compound according to an embodiment, a case where each of R₁ to R₄ is a substituted or unsubstituted pyridine group, a substituted or unsubstituted pyrimidine group, a substituted or unsubstituted pyridazine group, a substituted or unsubstituted pyrazine group, or a substituted or unsubstituted 1,3,5-triazine group may be excluded. In other words, R₁ to R₄ may not each be a substituted or unsubstituted pyridine group, a substituted or unsubstituted pyrimidine group, a substituted or unsubstituted pyridazine group, a substituted or unsubstituted pyrazine group, or a substituted or unsubstituted 1,3,5-triazine group.

In the amine compound of an embodiment, a case of Formula 2 where X₁ is O, a case where Formula 3 is represented by Formula 3-a is excluded. In other words, when X₁ is O, Formula 3 may not be represented by Formula 3-a. For example, in the amine compound of an embodiment, when the first substituent includes a dibenzofuran moiety, a case where Formula 3 is represented by Formula 3-a is excluded. In other words, when the first substituent includes a dibenzofuran moiety, Formula 3 may not be represented by Formula 3-a.

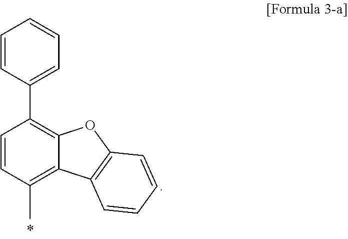 [SMILES: Cc1ccc(-c2ccccc2)c2oc3ccccc3c12]

In Formula 2, a is an integer of 0 to 2. In Formula 2, when a is 0, the amine compound of an embodiment may be unsubstituted with R₁. In Formula 2, a case where a is 2, and all R₁ are hydrogen atoms, may be the same as a case where a in Formula 2 is 0. When a is 2, multiple R₁ may be all the same, or at least one among multiple R₁ may be different.

In Formula 2 and Formula 3, b and c may each independently be an integer of 0 to 4. In Formula 2 and Formula 3, when b and c are 0, the amine compound of an embodiment may be unsubstituted with R₂ and R₃, respectively. In Formula 2 and Formula 3, a case where each of b and c is 4, and each of all R₂ and R₃ are hydrogen atoms, may be the same as a case where each of b and c in Formula 2 and Formula 3 is 0, respectively. When b and c are 2, each of multiple R₂ and R₃ may be all the same, or at least one among each of multiple R₂ and R₃ may be different.

In Formula 3, d is an integer of 0 to 3. In Formula 3, when d is 0, the amine compound of an embodiment may be unsubstituted with R₄. In Formula 3, a case where d is 3, and all R₄ are hydrogen atoms, may be the same as a case where d in Formula 3 is 0. When d is an integer of 2 or more, multiple R₄ may be all the same, or at least one among multiple R₄ may be different.

In an embodiment, the amine compound represented by Formula 1 may be represented by Formula 1-1.

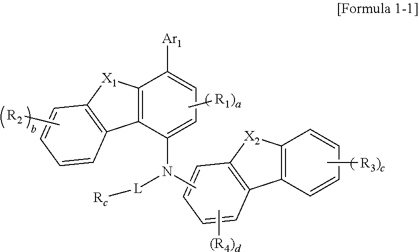

Formula 1-1 represents a case of Formula 1 where the substitution position of the substituent represented by Ar₁ in R_{A} represented by Formula 2, is specified.

In Formula 1-1, the same definitions or contents as those explained in Formula 1, Formula 2 and Formula 3 may be applied for R_{C}, L, Ar₁, X₁, X₂, R₁ to R₄, and a to d.

In the amine compound of an embodiment, the first substituent may further include a second aryl group in addition to the first aryl group. In the amine compound of an embodiment, the first substituent may be connected to or combined with the core nitrogen atom at carbon position 1, the first aryl group may be connected to (i.e., substituted in) at least one among carbon atoms at positions 2 to 4 of the first substituent, and the second aryl group may be connected to (i.e., substituted in) at least one among carbon atoms at positions 5 to 8 of the first substituent.

In an embodiment, the amine compound represented by Formula 1 may be represented by Formula 1-2-1 or Formula 1-2-2.

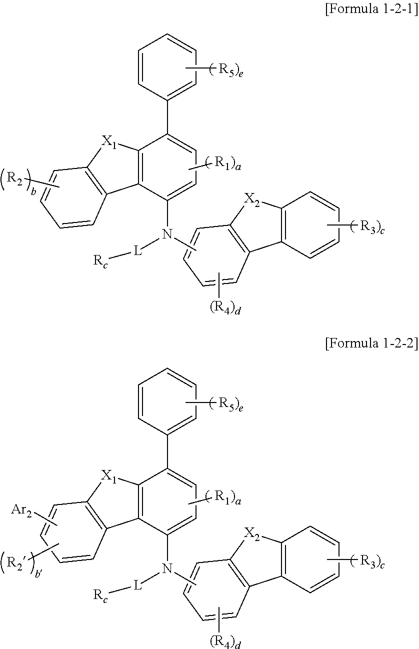

Formula 1-2-1 represents a case of Formula 1 where the type or kind and substitution position of the substituent represented by Ar₁ in R_{A} represented by Formula 2 are described or specified. Formula 1-2-2 represents a case of Formula 1 where the type or kind and substitution position of the substituent represented by Ar₁ in R_{A} represented by Formula 2 are described or specified, and the type or kind of the substituent represented by R₂ are described or specified. In some embodiments, in Formula 1-2-2, the substituent represented by Ar₂ may be a substituent corresponding to the second aryl group as described herein.

In Formula 1-2-1 and Formula 1-2-2, R₂′ and R₅ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 1 ring-forming carbon atoms. For example, R₂′ and R₅ may each independently be a hydrogen atom, or a deuterium atom.

In Formula 1-2-2, Ar₂ may be a substituted or unsubstituted aryl group of 6 to 30 ring-forming carbon atoms, or a substituted or unsubstituted heteroaryl group of 2 to 30 ring-forming carbon atoms. For example, Ar₂ may be a substituted or unsubstituted phenyl group.

In Formula 1-2-2, b′ may be an integer of 0 to 3. In Formula 1-2-2, when b′ is 0, the amine compound of an embodiment may be unsubstituted with R₂′. In Formula 1-2-2, a case where b′ is 3, and all R₂′ are hydrogen atoms, may be the same as a case where b′ in Formula 1-2-2 is 0. When b′ is an integer of 2 or more, multiple R₂′ may be all the same, or at least one among multiple R₂′ may be different.

In Formula 1-2-1 and Formula 1-2-2, e may be an integer of 0 to 5. In Formula 1-2-1 and Formula 1-2-2, when e is 0, the amine compound of an embodiment may be unsubstituted with R₅. In Formula 1-2-1 and Formula 1-2-2, a case where e is 5, and all R₅ are hydrogen atoms, may be the same as a case where e in Formula 1-2-1 and Formula 1-2-2 is 0. When e is an integer of 2 or more, multiple R₅ may be all the same, or at least one among multiple R₅ may be different.

In Formula 1-2-1 and Formula 1-2-2, the same definitions or contents as those explained in Formula 1, Formula 2, and Formula 3 may be applied for R_{C}, L, X₁, X₂, R₁ to R₄, and a to d.

In an embodiment, the substituent represented by Formula R_{C} may be represented by any one selected from among Formula A-1 to Formula A-8.

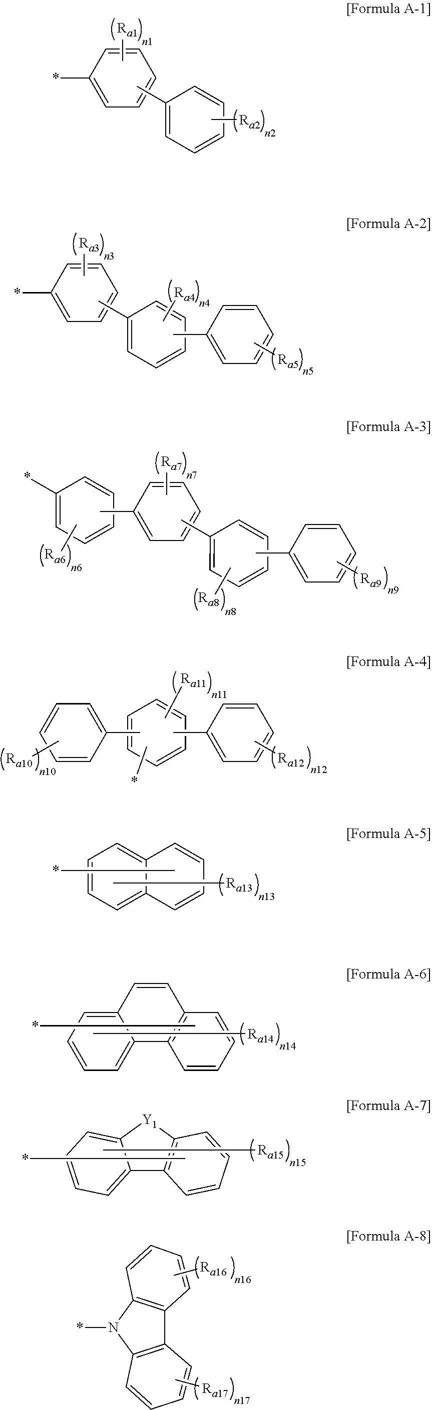

In Formula A-7, Y1 may be O, S, CRₐ₁₈Rₐ₁₉, or NRₐ₂₀.

In Formula A-1 to Formula A-8, Rₐ₁ to Rₐ₁₉ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Rₐ₁ to Rₐ₁₉ may each independently be a hydrogen atom, a deuterium atom, or a substituted or unsubstituted phenyl group.

In Formula A-7, Rₐ₂₀ may be a substituted or unsubstituted aryl group of 6 to 30 ring-forming carbon atoms, or a substituted or unsubstituted heteroaryl group of 2 to 30 ring-forming carbon atoms. For example, Rₐ₂₀ may be a substituted or unsubstituted phenyl group.

In Formula A-1 to Formula A-3, n1, n3, n4, and n6 to n8 may each independently be an integer of 0 to 4. In Formula A-1 to Formula A-3, when n1, n3, n4, and n6 to n8 are 0, the amine compound of an embodiment may be unsubstituted with Rₐ₁, Rₐ₃, Rₐ₄, and Rₐ₆ to Rₐ₈, respectively. In Formula A-1 to Formula A-3, a case where each of n1, n3, n4, and n6 to n8 is 4, and each of all Rₐ₁, Rₐ₃, Rₐ₄, and Rₐ₆ to Rₐ₈ are hydrogen atoms, may be the same as a case where each of n1, n3, n4, and n6 to n8 in Formula A-1 to Formula A-3 is 0, respectively. When each of n1, n3, n4, and n6 to n8 is an integer of 2 or more, each of multiple Rₐ₁, Rₐ₃, Rₐ₄, and Rₐ₆ to Rₐ₈ may be all the same, or at least one among each of multiple Rₐ₁, Rₐ₃, Rₐ₄, and Rₐ₆ to Rₐ₈ may be different, respectively.

In Formula A-1 to Formula A-4, n2, n5, n9, n10, and n12 may each independently be an integer of 0 to 5. In Formula A-1 to Formula A-4, when n2, n5, n9, n10, and n12 are 0, the amine compound of an embodiment may be unsubstituted with Rₐ₂, Rₐ₅, Rₐ₉, Rₐ₁₀, and Rₐ₁₂, respectively. In Formula A-1 to Formula A-4, a case where each of n2, n5, n9, n10, and n12 is 5, and each of all Rₐ₂, Rₐ₅, Rₐ₉, Rₐ₁₀, and Rₐ₁₂ are hydrogen atoms, may be the same as a case where each of n2, n5, n9, n10, and n12 in Formula A-1 to Formula A-4 is 0, respectively. When each of n2, n5, n9, n10, and n12 is an integer of 2 or more, each of multiple Rₐ₂, Rₐ₅, Rₐ₉, Rₐ₁₀, and Rₐ₁₂ may be all the same, or at least one among each of multiple Rₐ₂, Rₐ₅, Rₐ₉, Rₐ₁₀, and Rₐ₁₂ may be different, respectively.

In Formula A-4, n11 may be an integer of 0 to 3. In Formula A-4, when n11 is 0, the amine compound of an embodiment may be unsubstituted with Rₐ₁₁. In Formula A-4, a case where n11 is 3, and all Rₐ₁₁ are hydrogen atoms, may be the same as a case where n11 in Formula A-4 is 0. When n11 is an integer of 2 or more, multiple Rₐ₁₁ may be all the same, or at least one among multiple Rₐ₁₁ may be different.

In Formula A-5 and Formula A-7, n13 and n15 may each independently be an integer of 0 to 7. In Formula A-5 and Formula A-7, when n13 and n15 are 0, the amine compound of an embodiment may be unsubstituted with Rₐ₁₃ and Rₐ₁₅, respectively. In Formula A-5 and Formula A-7, a case where each of n13 and n15 is 7, and each of all Rₐ₁₃ and Rₐ₁₅ are hydrogen atoms, may be the same as a case where each of n13 and n15 in Formula A-5 and Formula A-7 is 0, respectively. When n13 and n15 are integers of 2 or more, each of multiple Rₐ₁₃ and Rₐ₁₅ may be all the same, or at least one among each of multiple Rₐ₁₃ and Rₐ₁₅ may be different, respectively.

In Formula A-6, n14 may be an integer of 0 to 9. In Formula A-6, when n14 is 0, the amine compound of an embodiment may be unsubstituted with Rₐ₁₄. In Formula A-6, a case where n14 is 9, and all Rₐ₁₄ are hydrogen atoms, may be the same as a case where n14 in Formula A-6 is 0. When n14 is an integer of 2 or more, multiple Rₐ₁₄ may be all the same, or at least one among multiple Rₐ₁₄ may be different.

In Formula A-8, n16 and n17 may each independently be an integer of 0 to 4. In Formula A-8, when n16 and n17 are 0, the amine compound of an embodiment may be unsubstituted with Rₐ₁₆ and Rₐ₁₇, respectively. In Formula A-8, a case where each of n16 and n17 is 4, and each of all Rₐ₁₆ and Rₐ₁₇ are hydrogen atoms, may be the same as a case where each of n16 and n17 in Formula A-8 is 0, respectively. When n16 and n17 are integers of 2 or more, each of multiple Rₐ₁₆ and Rₐ₁₇ may be all the same, or at least one among each of multiple Rₐ₁₆ and Rₐ₁₇ may be different, respectively.

In Formula A-1 to Formula A-8, -* is a position connected with L of Formula 1.

In an embodiment, the amine compound represented by Formula 1 may be represented by any one selected from among Formula 1-4-1 to Formula 1-4-3.

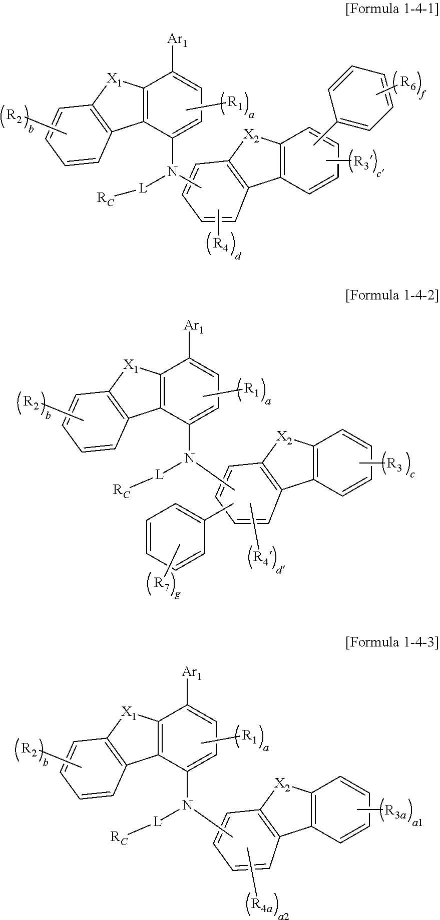

Formula 1-4-1 to Formula 1-4-3 represent Formula 1 where the substitution position of the substituent represented by Ar₁ in R_{A} represented by Formula 2 is described or specified, and the types (kinds) of the substituents represented by R₃ and/or R₄ of Formula 3 are described or specified.

In Formula 1-4-1 and Formula 1-4-2, R₃′, R₄′, R₆ and R₇ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₃′, R₄′, R₆ and R₇ may be hydrogen atoms or deuterium atoms.

In Formula 1-4-3, R₃ₐ and R₄ₐ may each independently be a hydrogen atom, or a deuterium atom. For example, R₃ₐ and R₄ₐ may be hydrogen atoms.

In Formula 1-4-1 and Formula 1-4-2, f and g may each independently be an integer of 0 to 5. In Formula 1-4-1 and Formula 1-4-2, when f and g are 0, the amine compound of an embodiment may be unsubstituted with R₆ and R₇, respectively. In Formula 1-4-1 and Formula 1-4-2, a case where each of f and g is 5, and each of all R₆ and R₇ are hydrogen atoms, may be the same as a case where each of f and g in Formula 1-4-1 and Formula 1-4-2 is 0, respectively. When f and g are integers of 2 or more, each of multiple R₆ and R₇ may be all the same, or at least one among each of multiple R₆ and R₇ may be different, respectively.

In Formula 1-4-1 and Formula 1-4-3, c′ and a2 may each independently be an integer of 0 to 3. In Formula 1-4-1 and Formula 1-4-3, when c′ and a2 are 0, the amine compound of an embodiment may be unsubstituted with R₃′ and R₄ₐ, respectively. In Formula 1-4-1 and Formula 1-4-3, a case where each of c′ and a2 is 3, and each of all R₃′ and R₄ₐ are hydrogen atoms, may be the same as a case where each of c′ and a2 in Formula 1-4-1 and Formula 1-4-3 is 0, respectively. When c′ and a2 are integers of 2 or more, each of multiple R₃′ and R₄ₐ may be all the same, or at least one among each of multiple R₃′ and R₄ₐ may be different, respectively.

In Formula 1-4-2, d′ may be an integer of 0 to 2. In Formula 1-4-2, when d′ is 0, the amine compound of an embodiment may be unsubstituted with R₄′. In Formula 1-4-2, a case where d′ is 2, and all R₄′ are hydrogen atoms, may be the same as a case where d′ in Formula 1-4-2 is 0. When d′ is 2, multiple R₄′ may be all the same, or at least one among multiple R₄′ may be different.

In Formula 1-4-3, a1 may be an integer of 0 to 4. In Formula 1-4-3, when a1 is 0, the amine compound of an embodiment may be unsubstituted with R₄ₐ. In Formula 1-4-3, a case where a1 is 4, and all R₄ₐ are hydrogen atoms, may be the same as a case where a1 in Formula 1-4-3 is 0. When a1 is 2 or more, multiple R₄ₐ may be all the same, or at least one among multiple R₄ₐ may be different.

In Formula 1-4-1 to Formula 1-4-3, the same definitions or contents as those explained in Formula 1, Formula 2, and Formula 3 may be applied for R_{C}, L, Ar₁, X₁, X₂, R₁ to R₄, and a to d.

In an embodiment, the amine compound represented by Formula 1 may be represented by Formula 1-5-1 or Formula 1-5-2.

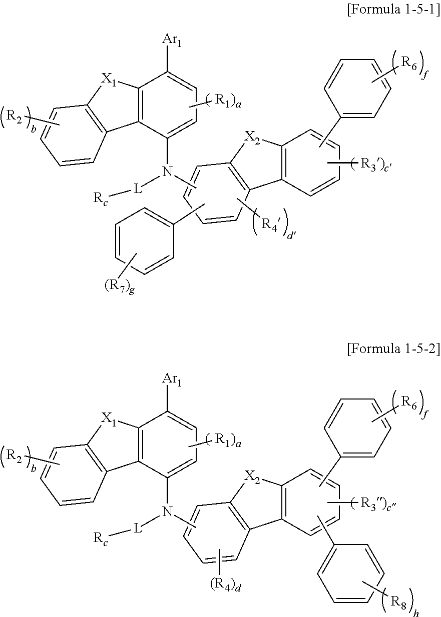

Formula 1-5-1 and Formula 1-5-2 represent Formula 1 where the substitution position of the substituent represented by Ar₁ in R_{A} represented by Formula 2 is described or specified, and the types (kinds) of the substituents represented by R₃ and/or R₄ of Formula 3 are described or specified.

In Formula 1-5-1 and Formula 1-5-2, R₃′, R₃″, R₄′, and R₆ to R₈ may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₃′, R₃″, R₄′, and R₆ to R₈ may each independently be a hydrogen atom or a deuterium atom.

In Formula 1-5-1 and Formula 1-5-2, f to h may each independently be an integer of 0 to 5. In Formula 1-5-1 and Formula 1-5-2, when f to h are 0, the amine compound of an embodiment may be unsubstituted with R₆ to R₈, respectively. In Formula 1-5-1 and Formula 1-5-2, a case where each of f to h is 5, and each of all R₆ to R₈ are hydrogen atoms, may be the same as a case where each of f to h in Formula 1-5-1 and Formula 1-5-2 is 0, respectively. When f to h are integers of 2 or more, each of multiple R₆ to R₈ may be all the same, or at least one among each of multiple R₆ to R₈ may be different, respectively.

In Formula 1-5-1, c′ is an integer of 0 to 3. In Formula 1-5-1, when c′ is 0, the amine compound of an embodiment may be unsubstituted with R₃′. In Formula 1-5-1, a case where c′ is 3, and all R₃′ are hydrogen atoms, may be the same as a case where c′ in Formula 1-5-1 is 0. When c′ is an integer of 2 or more, multiple R₃′ may be all the same, or at least one among multiple R₃′ may be different.

In Formula 1-5-1 and Formula 1-5-2, c″ and d′ may each independently be an integer of 0 to 2. In Formula 1-5-1 and Formula 1-5-2, when c″ and d′ are 0, the amine compound of an embodiment may be unsubstituted with R₃″ and R₄′, respectively. In Formula 1-5-1 and Formula 1-5-2, a case where each of c″ and d′ is 2, and each of all R₃″ and R₄′ are hydrogen atoms, may be the same as a case where each of c″ and d′ in Formula 1-5-1 and Formula 1-5-2 are 0, respectively. When c″ and d′ are 2, each of multiple R₃″ and R₄′ may be all the same, or at least one among each of multiple R₃″ and R₄′ may be different, respectively.

In Formula 1-5-1 and Formula 1-5-2, the same definitions or contents as those explained in Formula 1, Formula 2, and Formula 3 may be applied for R_{C}, L, Ar₁, X₁, X₂, R₁ R₂, R₄, a, b, and d.

In an embodiment, the amine compound represented by Formula 1 may be represented by any one selected from among Formula 1-6-1 to Formula 1-6-4.

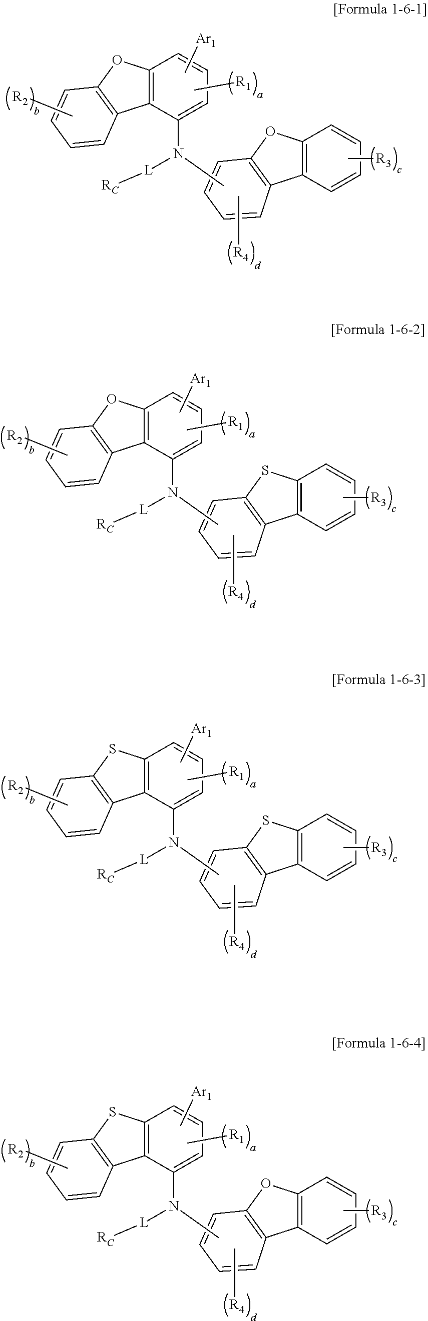

Formula 1-6-1 to Formula 1-6-4 represent Formula 1 where X₁ in R_{A} represented by Formula 2 and X₂ in R_{B} represented by Formula 3 are defined or specified as O or S. Formula 1-6-1 is a case of Formula 1 where both (e.g., simultaneously) X₁ of Formula 2 and X₃ of Formula 3 are O, Formula 1-6-2 is a case of Formula 1 where X₁ of Formula 2 is 0, and X₂ of Formula 3 is S, Formula 1-6-3 is a case of Formula 1 where both (e.g., simultaneously) X₁ of Formula 2 and X₂ of Formula 3 are S, and Formula 1-6-4 is a case of Formula 1 where X₁ of Formula 2 is S, and X₂ of Formula 3 is O.

In Formula 1-6-1 to Formula 1-6-4, the same definitions ore contents as those explained in Formula 1, Formula 2, and Formula 3 may be applied for R_{C}, L, Ar₁, R₁ to R₄, and a to d.

In an embodiment, the amine compound represented by Formula 1 may be represented by Formula 1-7-1 or Formula 1-7-2.

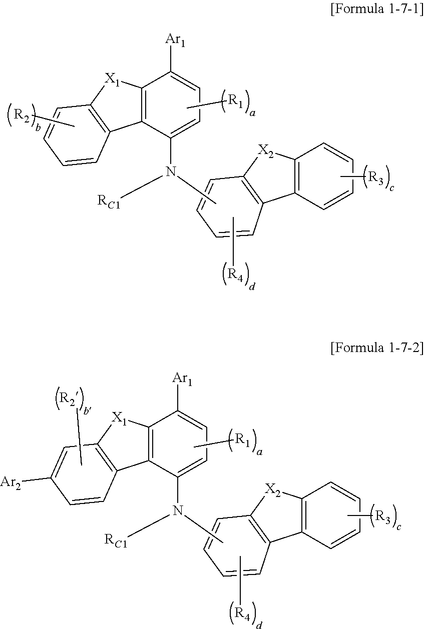

Formula 1-7-1 represents a case of Formula 1 where the substitution position of the substituent represented by Ar₁ of Formula 2 is described or specified, L is defined or specified as a direct linkage, and the type or kind of the substituent represented by R_{C} is defined or specified. Formula 1-7-2 represents a case of Formula 1 where the substitution position of the substituent represented by Ar₁ of Formula 2 is described or specified, the type or kind of the substituent represented by R₂ is described or specified, L is specified as a direct linkage, and the type or kind of the substituent represented by R_{C} is described or specified.

In Formula 1-7-2, R₂′ may be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R₂′ may be a hydrogen atom.

In Formula 1-7-2, Ar₂ may be a substituted or unsubstituted aryl group of 6 to 30 ring-forming carbon atoms, or a substituted or unsubstituted heteroaryl group of 2 to 30 ring-forming carbon atoms. For example, Ar₂ may be a substituted or unsubstituted phenyl group.

In Formula 1-7-2, b′ may be an integer of 0 to 3. In Formula 1-7-2, when b′ is 0, the amine compound of an embodiment may be unsubstituted with R₂′. In Formula 1-7-2, a case where b′ is 3, and all R₂′ are hydrogen atoms, may be the same as a case where b′ in Formula 1-7-2 is 0. When b′ is an integer of 2 or more, multiple R₂′ may be all the same, or at least one among multiple R₂′ may be different.

In Formula 1-7-1 and Formula 1-7-2, R_{C1} may be selected from Compound Group A.

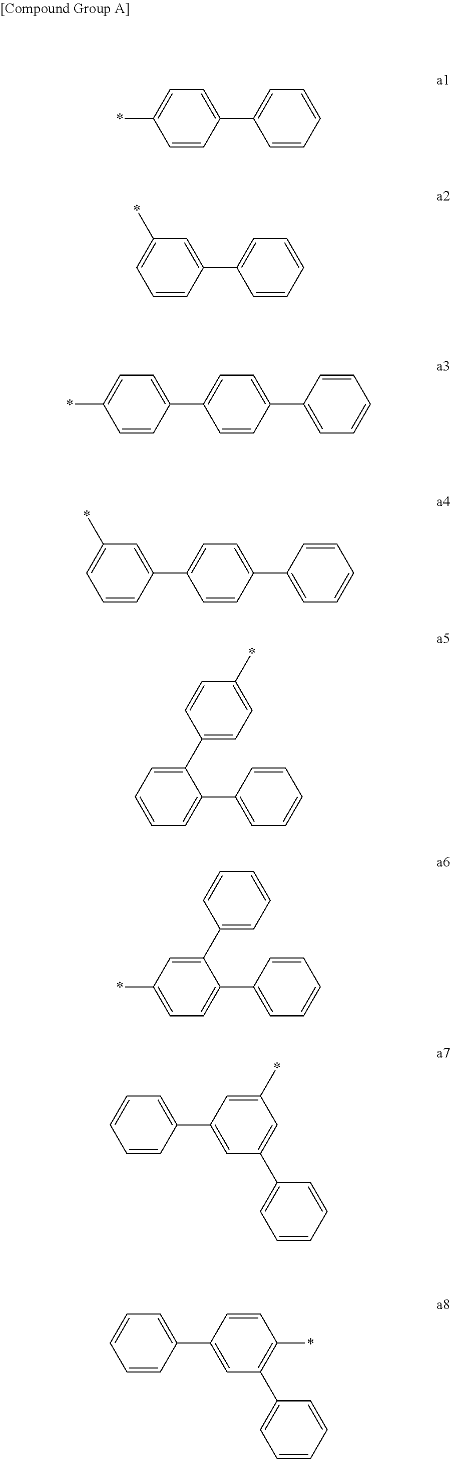

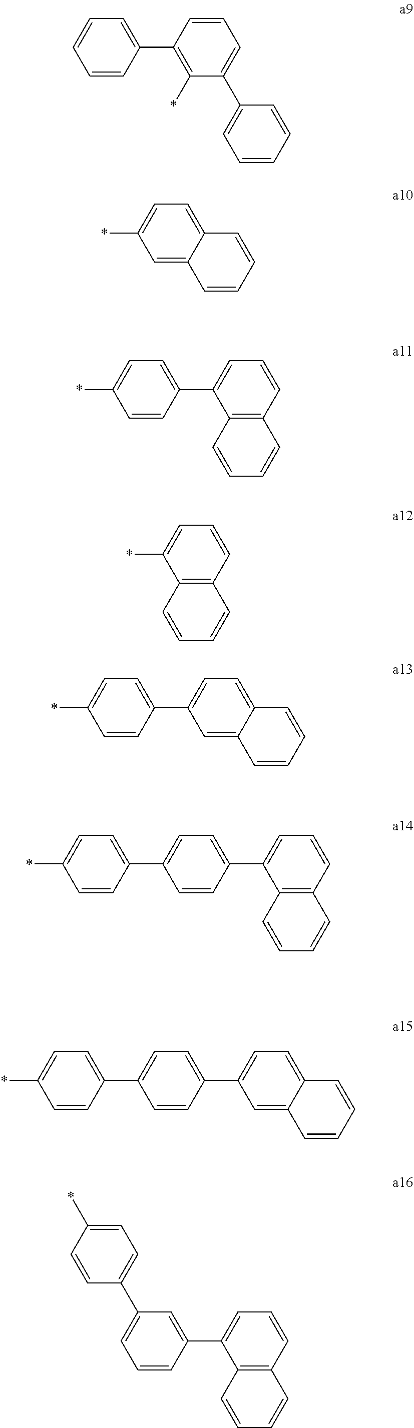

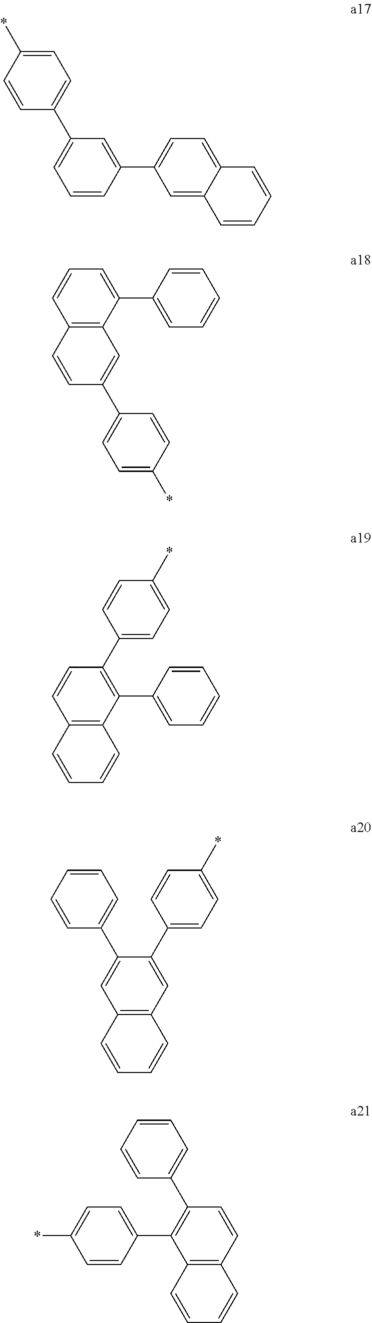 [SMILES: *c1ccc(-c2cccc(-c3ccc4ccccc4c3)c2)cc1 ; *c1ccc(-c2ccc3cccc(-c4ccccc4)c3c2)cc1 ; *c1ccc(-c2ccc3ccccc3c2-c2ccccc2)cc1 ; *c1ccc(-c2cc3ccccc3cc2-c2ccccc2)cc1 ; *c1ccc(-c2c(-c3ccccc3)ccc3ccccc23)cc1]

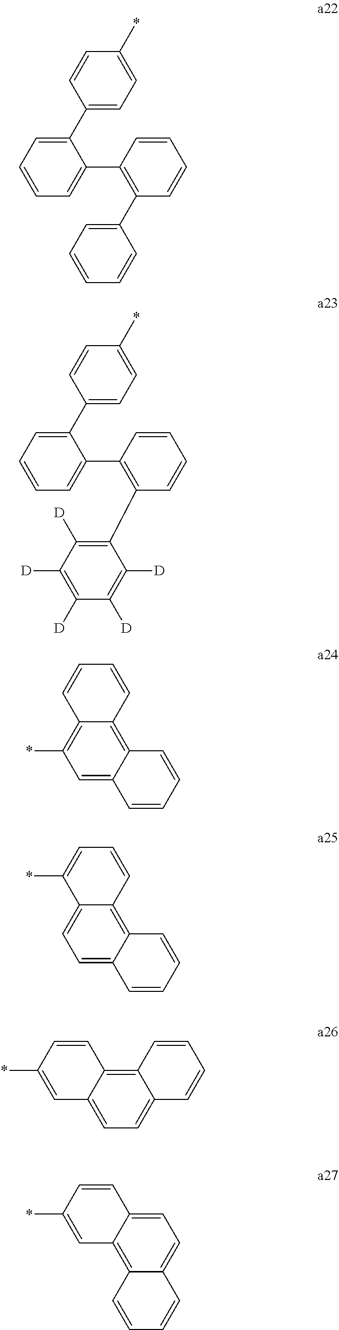

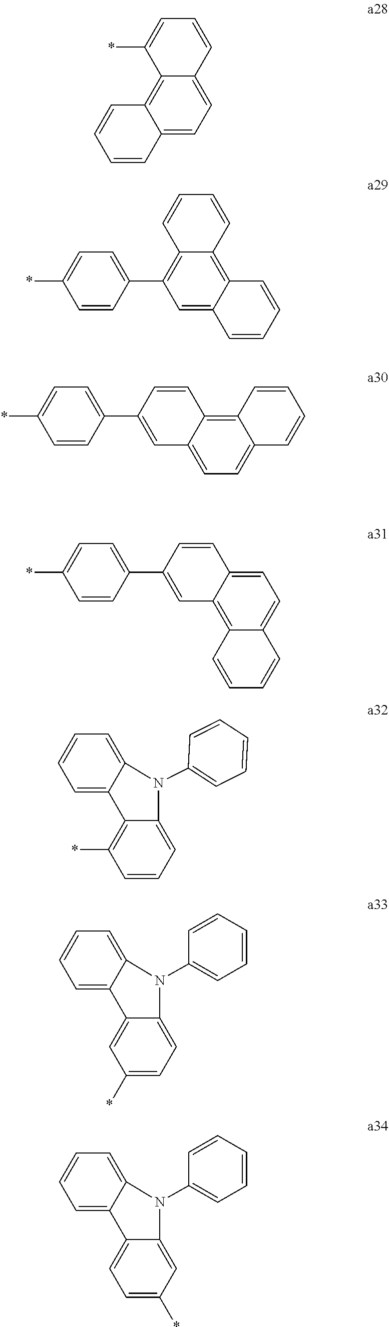

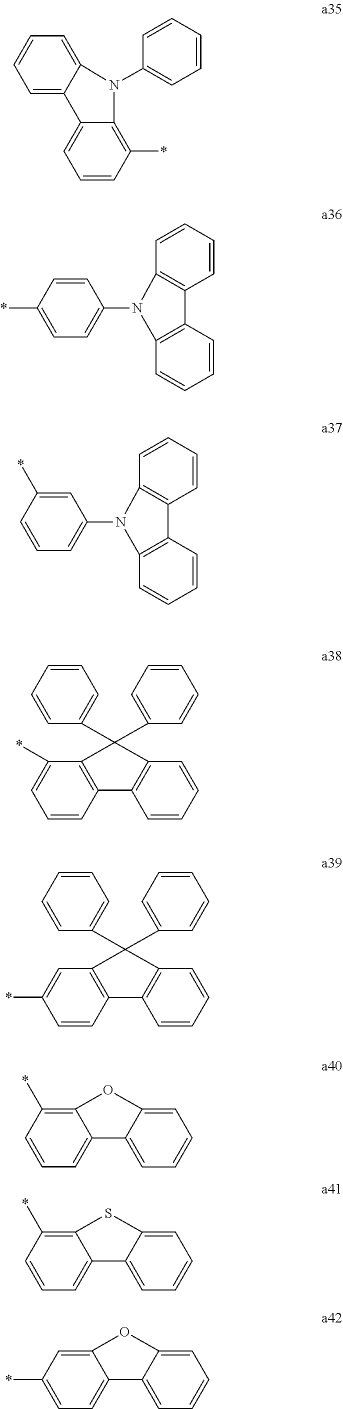

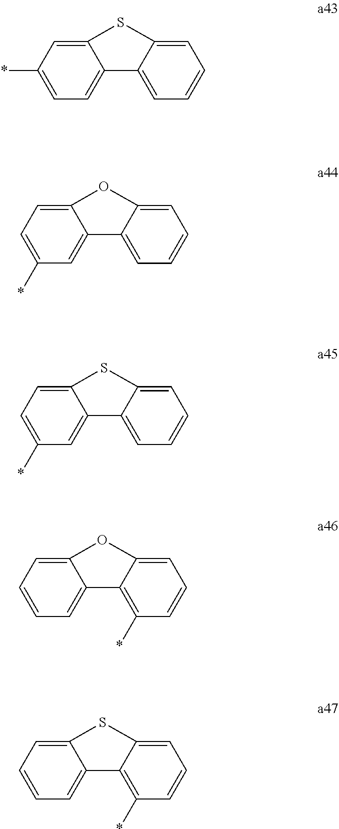 [SMILES: *c1ccc2c(c1)sc1ccccc12 ; *c1ccc2oc3ccccc3c2c1 ; *c1ccc2sc3ccccc3c2c1 ; *c1cccc2oc3ccccc3c12 ; *c1cccc2sc3ccccc3c12]

In Formula 1-7-1 and Formula 1-7-2, the same definitions or contents as those explained in Formula 1, Formula 2, and Formula 3 may be applied for Ar₁, X₁, X₂, R₁ to R₄, and a to d.

In an embodiment, L may be represented by any one selected from among Formula L-1 to Formula L-6.

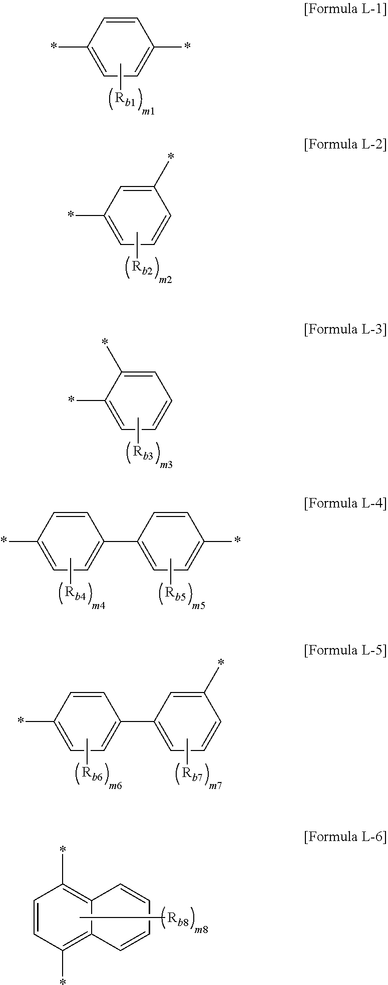

In Formula L-1 to Formula L-6, R_{b1} to R_{b8} may each independently be a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, R_{b1} to R_{b8} may be hydrogen atoms.

In Formula L-1 to Formula L-5, m1 to m7 may each independently be an integer of 0 to 4. In Formula L-1 to Formula L-5, when m1 to m7 are 0, the amine compound of an embodiment may be unsubstituted with R_{b1} to R_{b7}, respectively. In Formula L-1 to Formula L-5, a case where each of m1 to m7 is 4, and each of all R_{b1} to R_{b7} are hydrogen atoms, may be the same as a case where m1 to m7 in Formula L-1 to Formula L-5 are 0, respectively. When m1 to m7 are integers of 2 or more, each (of the multiple R_{b(each of 1-7)}) of the multiple R_{b1} to the multiple R_{b7} may be all the same, or at least one (among the multiple R_{b(one of 1-7)}) among any of (e.g., among each of) the multiple R_{b1} to the multiple R_{b7} may be different.

In Formula L-6, m8 may be an integer of 0 to 6. In Formula L-6, when m8 is 0, the amine compound of an embodiment may be unsubstituted with R_{b8}. In Formula L-6, a case where m8 is 6, and all R_{b8} are hydrogen atoms, may be the same as a case where m8 in Formula L-6 is 0. When m8 is an integer of 2 or more, the multiple R_{b8} may be all the same, or at least one among the multiple R_{b8} may be different.

The amine compound of an embodiment may be represented by any one selected from among the compounds in Compound Group 1. The hole transport region HTR of the light emitting element ED of an embodiment may include at least one selected from among the amine compounds shown in Compound Group 1. For example, the hole transport layer HTL of the light emitting element ED may include at least one selected from among the amine compounds shown in Compound Group 1.

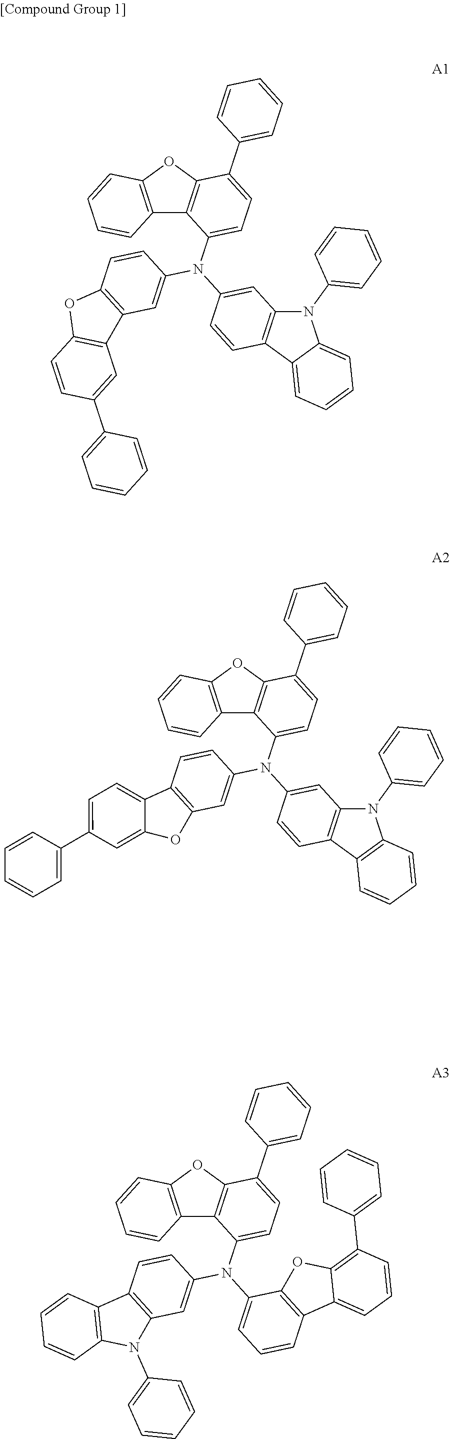 [SMILES: c1ccc(-c2ccc3oc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)oc2cc(N(c4ccc5c6ccccc6n(-c6ccccc6)c5c4)c4ccc(-c5ccccc5)c5oc6ccccc6c45)ccc23)cc1 ; c1ccc(-c2cccc3c2oc2c(N(c4ccc5c6ccccc6n(-c6ccccc6)c5c4)c4ccc(-c5ccccc5)c5oc6ccccc6c45)cccc23)cc1]

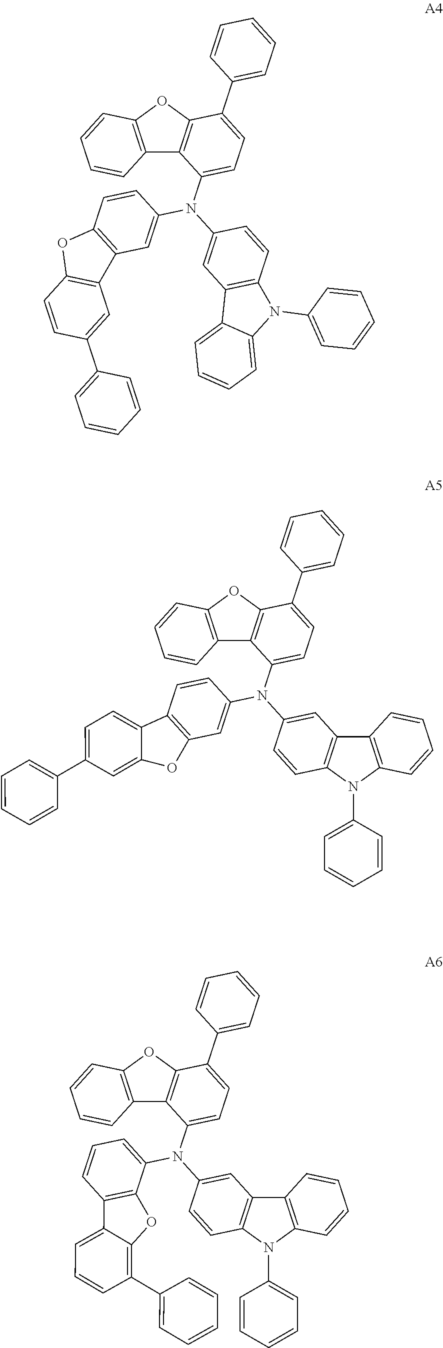 [SMILES: c1ccc(-c2ccc3oc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)oc2cc(N(c4ccc5c(c4)c4ccccc4n5-c4ccccc4)c4ccc(-c5ccccc5)c5oc6ccccc6c45)ccc23)cc1 ; c1ccc(-c2cccc3c2oc2c(N(c4ccc5c(c4)c4ccccc4n5-c4ccccc4)c4ccc(-c5ccccc5)c5oc6ccccc6c45)cccc23)cc1]

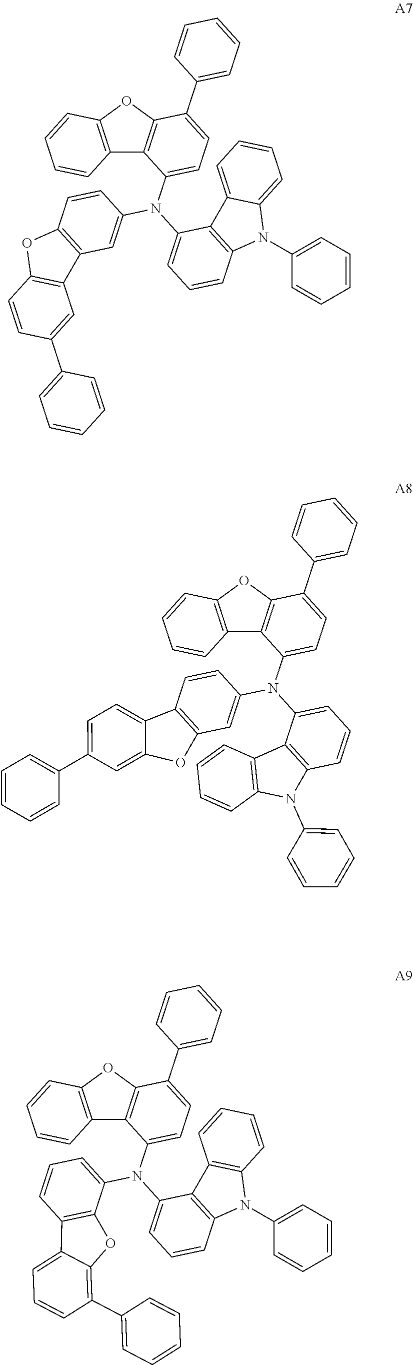 [SMILES: c1ccc(-c2ccc3oc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)oc2cc(N(c4ccc(-c5ccccc5)c5oc6ccccc6c45)c4cccc5c4c4ccccc4n5-c4ccccc4)ccc23)cc1 ; c1ccc(-c2cccc3c2oc2c(N(c4ccc(-c5ccccc5)c5oc6ccccc6c45)c4cccc5c4c4ccccc4n5-c4ccccc4)cccc23)cc1]

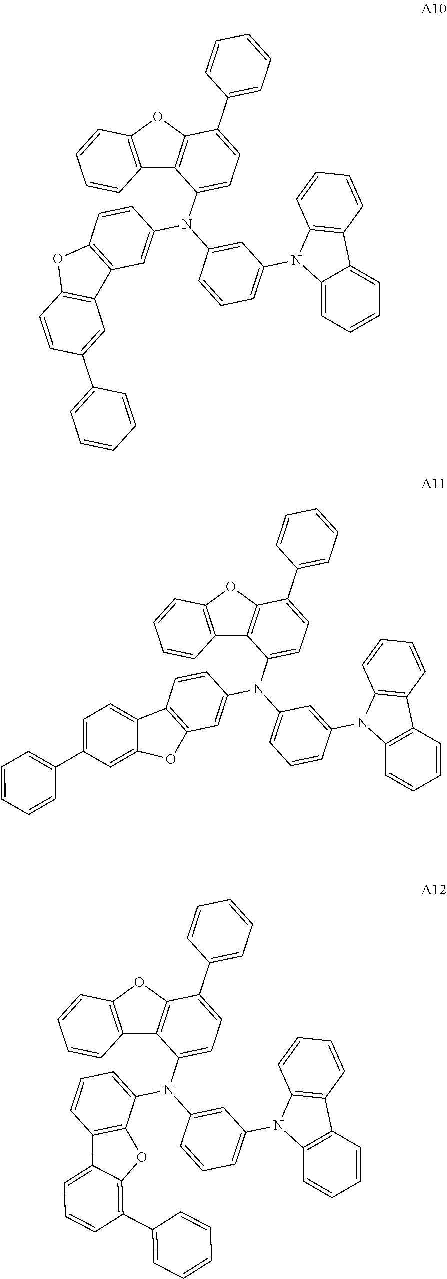 [SMILES: c1ccc(-c2ccc3oc4ccc(N(c5cccc(-n6c7ccccc7c7ccccc76)c5)c5ccc(-c6ccccc6)c6oc7ccccc7c56)cc4c3c2)cc1 ; c1ccc(-c2ccc3c(c2)oc2cc(N(c4cccc(-n5c6ccccc6c6ccccc65)c4)c4ccc(-c5ccccc5)c5oc6ccccc6c45)ccc23)cc1 ; c1ccc(-c2cccc3c2oc2c(N(c4cccc(-n5c6ccccc6c6ccccc65)c4)c4ccc(-c5ccccc5)c5oc6ccccc6c45)cccc23)cc1]

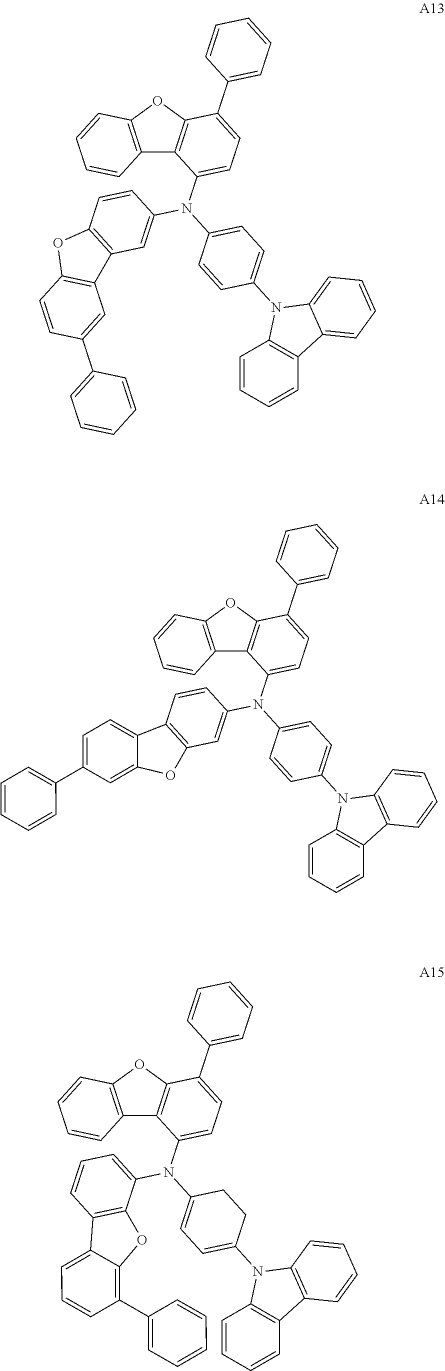 [SMILES: c1ccc(-c2ccc3oc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)oc2cc(N(c4ccc(-n5c6ccccc6c6ccccc65)cc4)c4ccc(-c5ccccc5)c5oc6ccccc6c45)ccc23)cc1 ; C1=C(N(c2cccc3c2oc2c(-c4ccccc4)cccc23)c2ccc(-c3ccccc3)c3oc4ccccc4c23)CCC(n2c3ccccc3c3ccccc32)=C1]

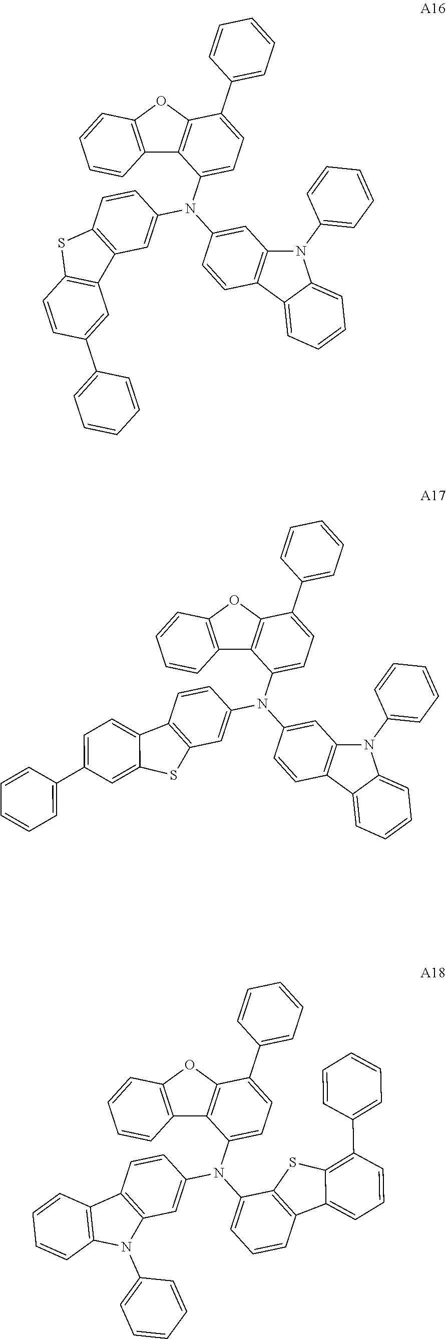 [SMILES: c1ccc(-c2ccc3sc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)sc2cc(N(c4ccc5c6ccccc6n(-c6ccccc6)c5c4)c4ccc(-c5ccccc5)c5oc6ccccc6c45)ccc23)cc1 ; c1ccc(-c2ccc(N(c3ccc4c5ccccc5n(-c5ccccc5)c4c3)c3cccc4c3sc3c(-c5ccccc5)cccc34)c3c2oc2ccccc23)cc1]

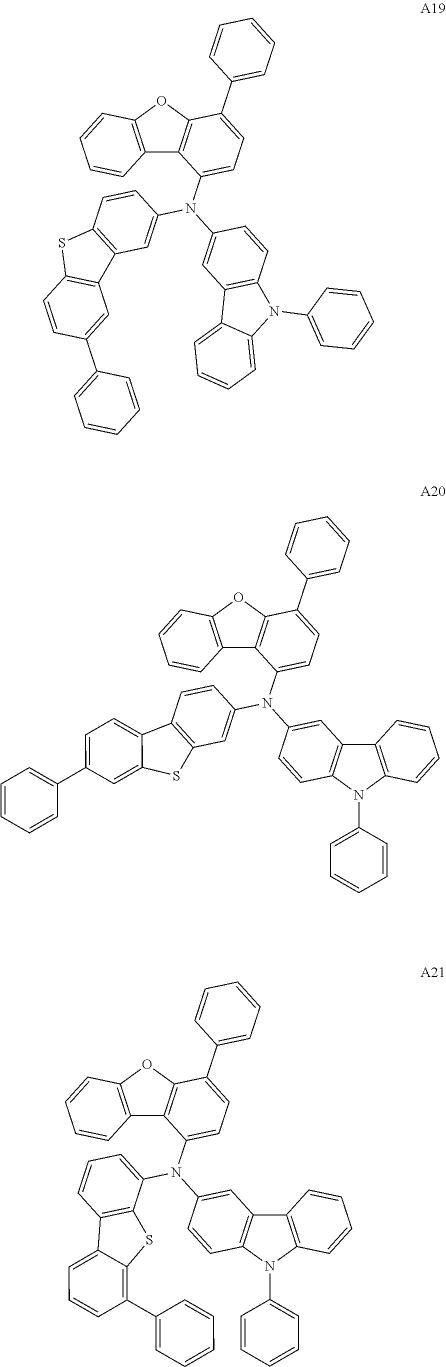 [SMILES: c1ccc(-c2ccc3sc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)sc2cc(N(c4ccc5c(c4)c4ccccc4n5-c4ccccc4)c4ccc(-c5ccccc5)c5oc6ccccc6c45)ccc23)cc1 ; c1ccc(-c2ccc(N(c3ccc4c(c3)c3ccccc3n4-c3ccccc3)c3cccc4c3sc3c(-c5ccccc5)cccc34)c3c2oc2ccccc23)cc1]

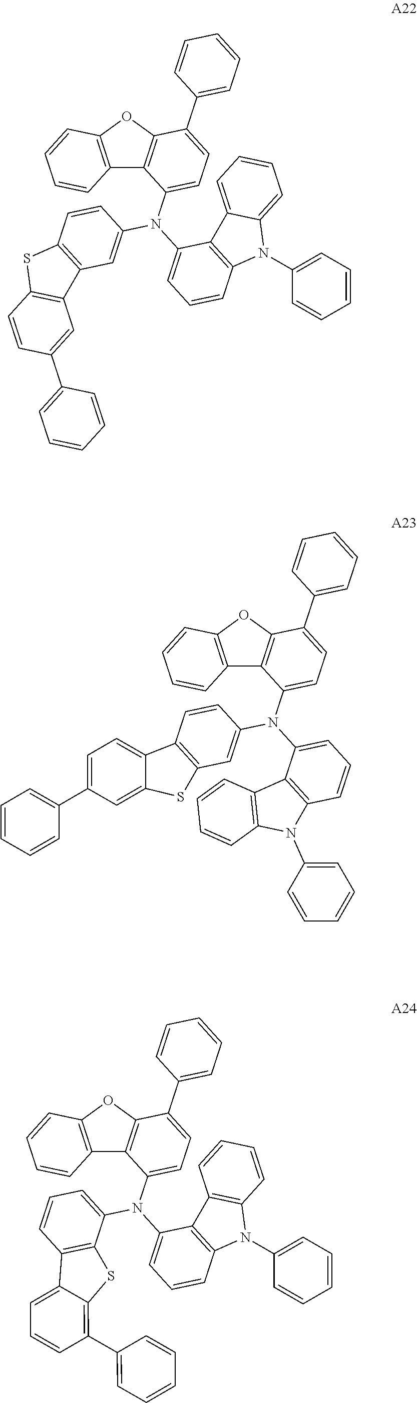 [SMILES: c1ccc(-c2ccc3sc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)sc2cc(N(c4ccc(-c5ccccc5)c5oc6ccccc6c45)c4cccc5c4c4ccccc4n5-c4ccccc4)ccc23)cc1 ; c1ccc(-c2ccc(N(c3cccc4c3sc3c(-c5ccccc5)cccc34)c3cccc4c3c3ccccc3n4-c3ccccc3)c3c2oc2ccccc23)cc1]

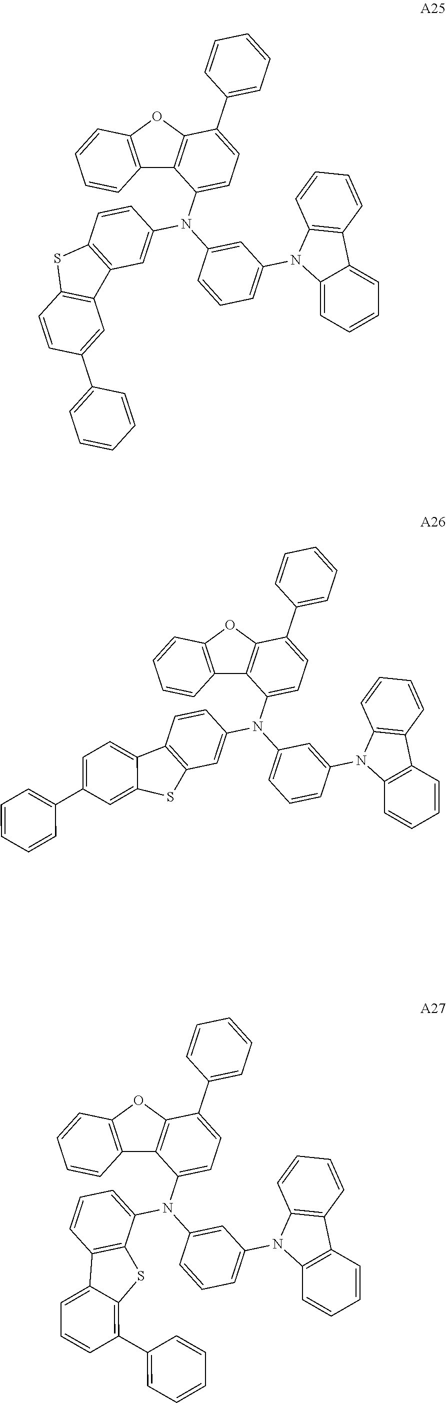 [SMILES: c1ccc(-c2ccc3sc4ccc(N(c5cccc(-n6c7ccccc7c7ccccc76)c5)c5ccc(-c6ccccc6)c6oc7ccccc7c56)cc4c3c2)cc1 ; c1ccc(-c2ccc3c(c2)sc2cc(N(c4cccc(-n5c6ccccc6c6ccccc65)c4)c4ccc(-c5ccccc5)c5oc6ccccc6c45)ccc23)cc1 ; c1ccc(-c2ccc(N(c3cccc(-n4c5ccccc5c5ccccc54)c3)c3cccc4c3sc3c(-c5ccccc5)cccc34)c3c2oc2ccccc23)cc1]

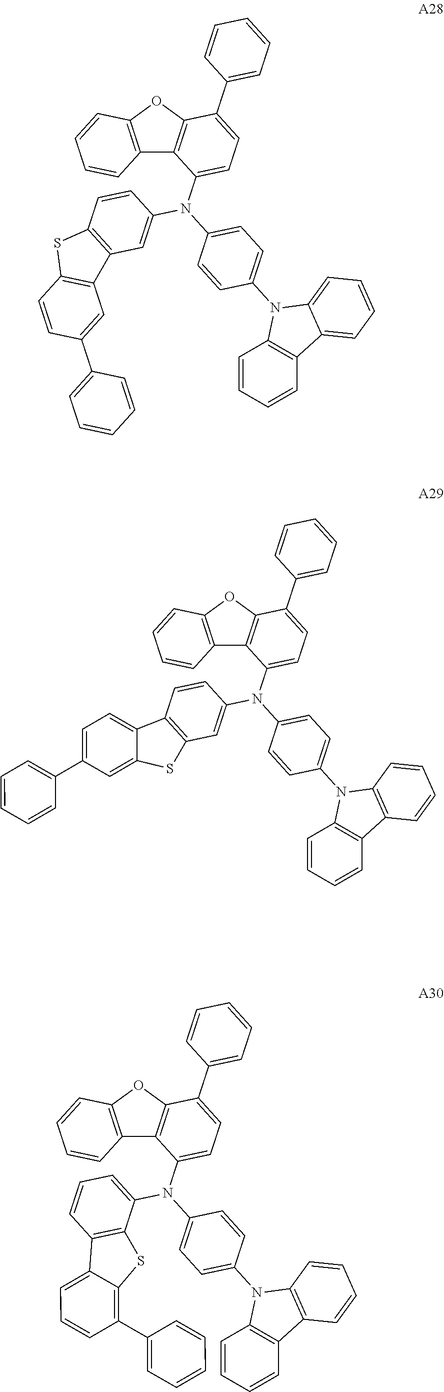 [SMILES: c1ccc(-c2ccc3sc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5oc6ccccc6c45)ccc23)cc1 ; c1ccc(-c2ccc(N(c3ccc(-n4c5ccccc5c5ccccc54)cc3)c3cccc4c3sc3c(-c5ccccc5)cccc34)c3c2oc2ccccc23)cc1]

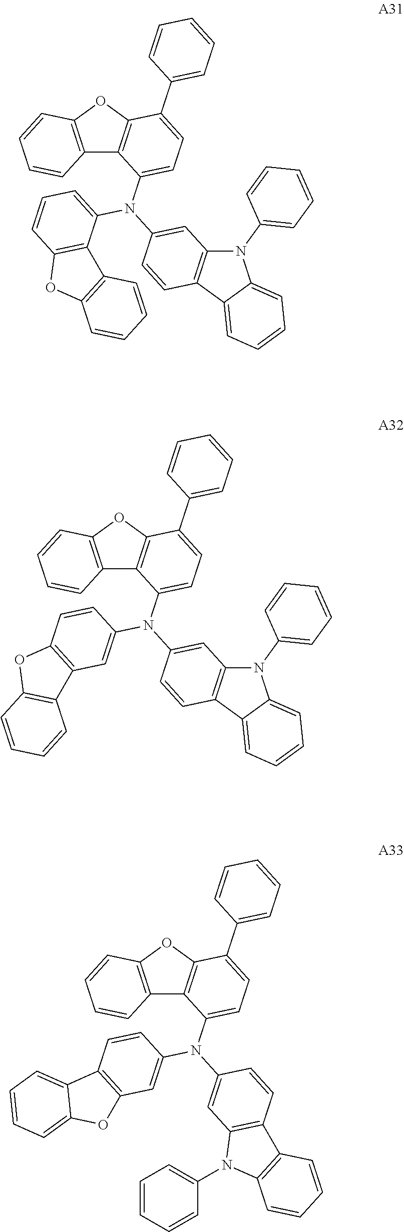 [SMILES: c1ccc(-c2ccc(N(c3ccc4c5ccccc5n(-c5ccccc5)c4c3)c3cccc4oc5ccccc5c34)c3c2oc2ccccc23)cc1 ; c1ccc(-c2ccc(N(c3ccc4oc5ccccc5c4c3)c3ccc4c5ccccc5n(-c5ccccc5)c4c3)c3c2oc2ccccc23)cc1 ; c1ccc(-c2ccc(N(c3ccc4c(c3)oc3ccccc34)c3ccc4c5ccccc5n(-c5ccccc5)c4c3)c3c2oc2ccccc23)cc1]

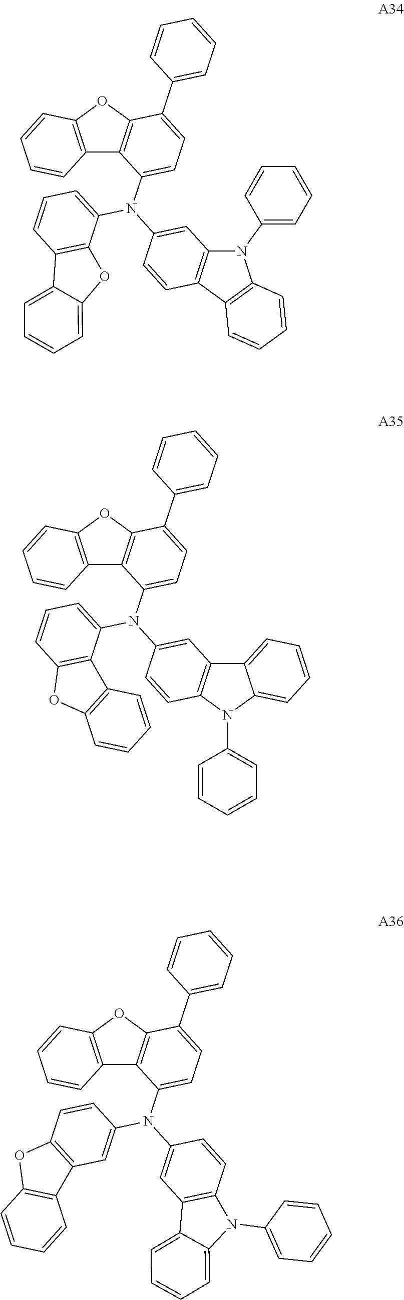 [SMILES: c1ccc(-c2ccc(N(c3ccc4c5ccccc5n(-c5ccccc5)c4c3)c3cccc4c3oc3ccccc34)c3c2oc2ccccc23)cc1 ; c1ccc(-c2ccc(N(c3ccc4c(c3)c3ccccc3n4-c3ccccc3)c3cccc4oc5ccccc5c34)c3c2oc2ccccc23)cc1 ; c1ccc(-c2ccc(N(c3ccc4oc5ccccc5c4c3)c3ccc4c(c3)c3ccccc3n4-c3ccccc3)c3c2oc2ccccc23)cc1]

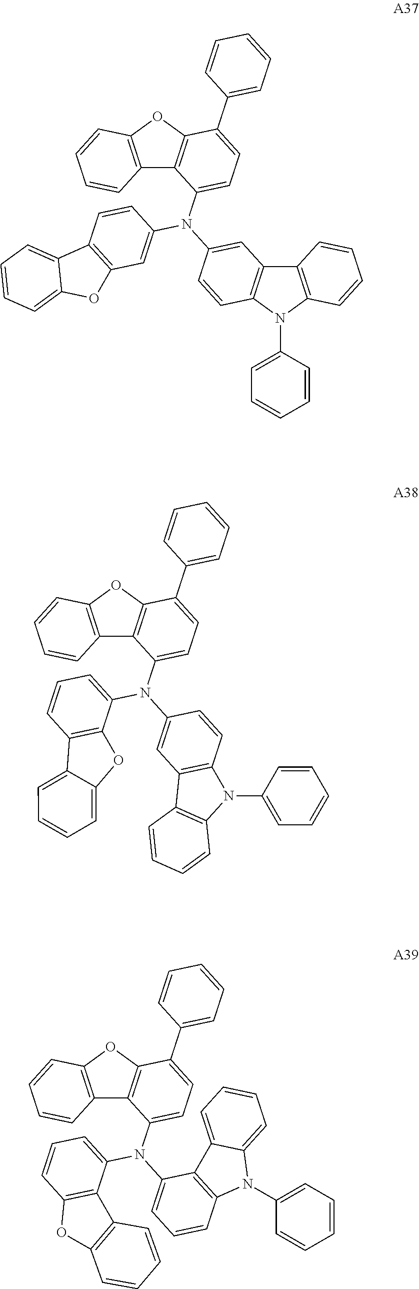 [SMILES: c1ccc(-c2ccc(N(c3ccc4c(c3)oc3ccccc34)c3ccc4c(c3)c3ccccc3n4-c3ccccc3)c3c2oc2ccccc23)cc1 ; c1ccc(-c2ccc(N(c3ccc4c(c3)c3ccccc3n4-c3ccccc3)c3cccc4c3oc3ccccc34)c3c2oc2ccccc23)cc1 ; c1ccc(-c2ccc(N(c3cccc4oc5ccccc5c34)c3cccc4c3c3ccccc3n4-c3ccccc3)c3c2oc2ccccc23)cc1]

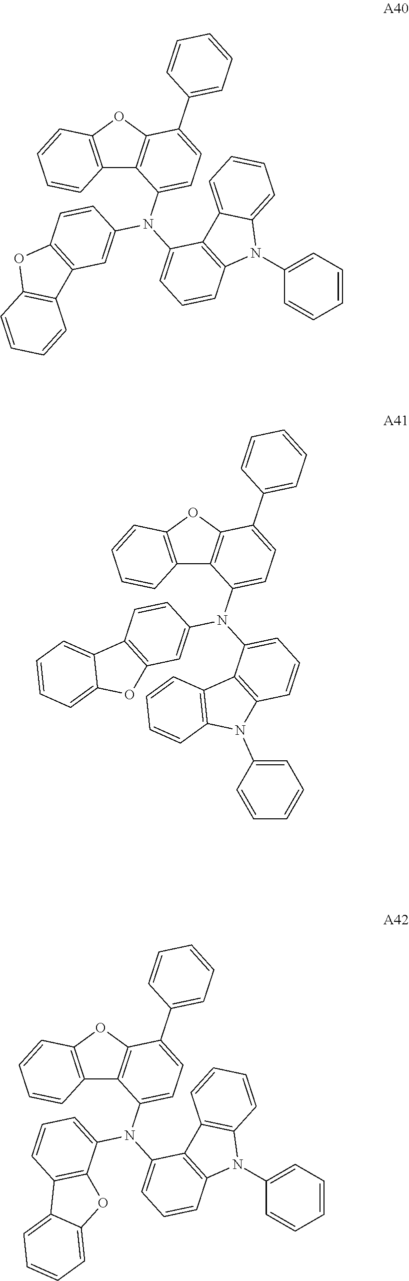 [SMILES: c1ccc(-c2ccc(N(c3ccc4oc5ccccc5c4c3)c3cccc4c3c3ccccc3n4-c3ccccc3)c3c2oc2ccccc23)cc1 ; c1ccc(-c2ccc(N(c3ccc4c(c3)oc3ccccc34)c3cccc4c3c3ccccc3n4-c3ccccc3)c3c2oc2ccccc23)cc1 ; c1ccc(-c2ccc(N(c3cccc4c3oc3ccccc34)c3cccc4c3c3ccccc3n4-c3ccccc3)c3c2oc2ccccc23)cc1]

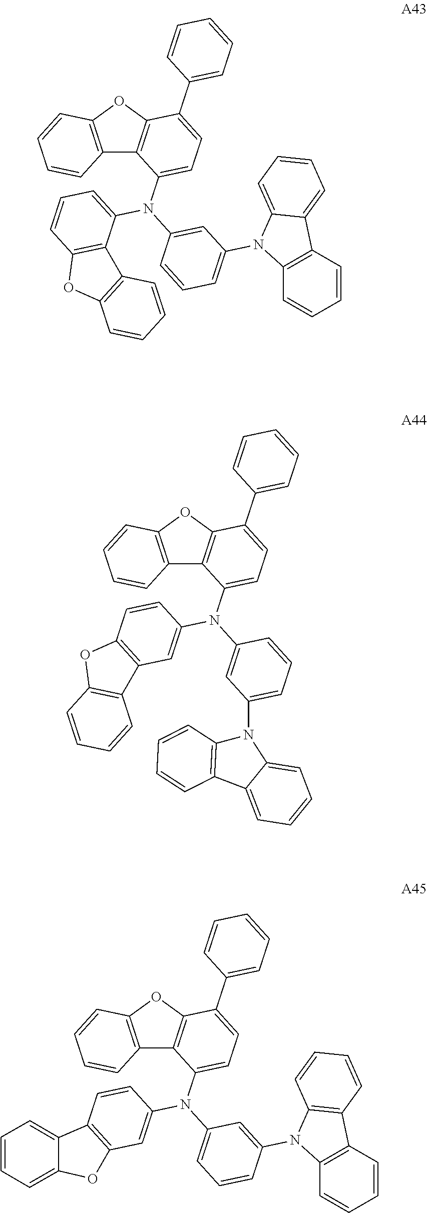 [SMILES: c1ccc(-c2ccc(N(c3cccc(-n4c5ccccc5c5ccccc54)c3)c3cccc4oc5ccccc5c34)c3c2oc2ccccc23)cc1 ; c1ccc(-c2ccc(N(c3cccc(-n4c5ccccc5c5ccccc54)c3)c3ccc4oc5ccccc5c4c3)c3c2oc2ccccc23)cc1 ; c1ccc(-c2ccc(N(c3cccc(-n4c5ccccc5c5ccccc54)c3)c3ccc4c(c3)oc3ccccc34)c3c2oc2ccccc23)cc1]

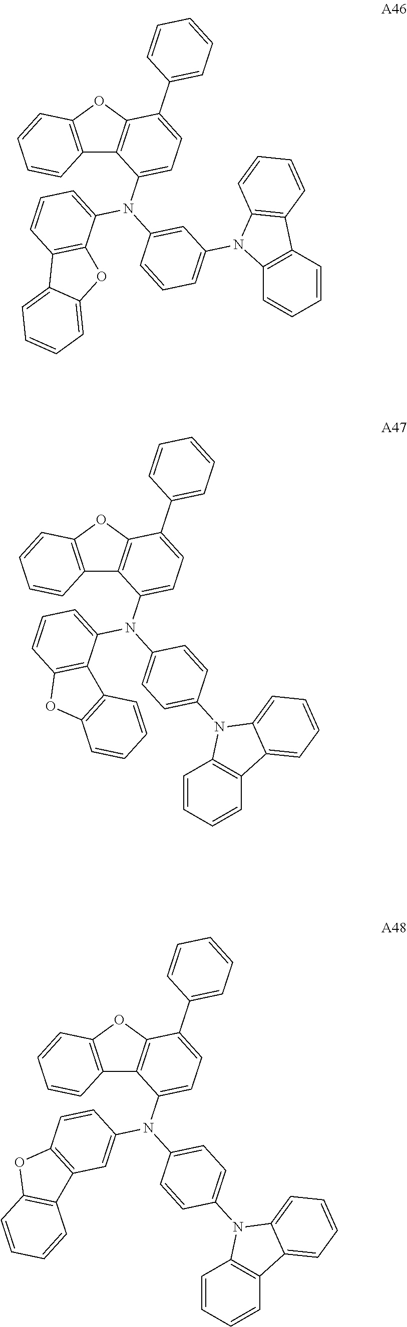 [SMILES: c1ccc(-c2ccc(N(c3cccc(-n4c5ccccc5c5ccccc54)c3)c3cccc4c3oc3ccccc34)c3c2oc2ccccc23)cc1 ; c1ccc(-c2ccc(N(c3ccc(-n4c5ccccc5c5ccccc54)cc3)c3cccc4oc5ccccc5c34)c3c2oc2ccccc23)cc1 ; c1ccc(-c2ccc(N(c3ccc(-n4c5ccccc5c5ccccc54)cc3)c3ccc4oc5ccccc5c4c3)c3c2oc2ccccc23)cc1]

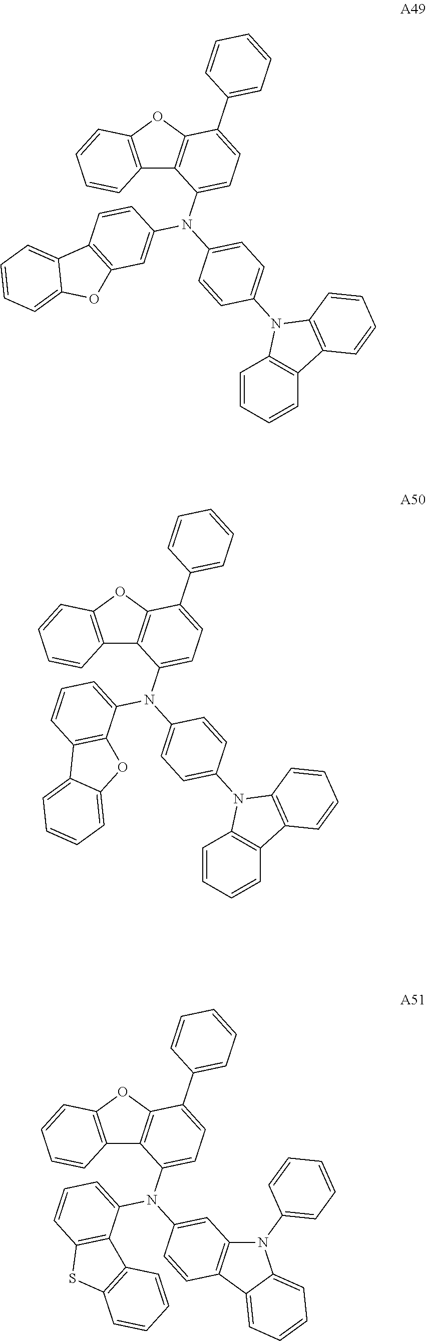 [SMILES: c1ccc(-c2ccc(N(c3ccc(-n4c5ccccc5c5ccccc54)cc3)c3ccc4c(c3)oc3ccccc34)c3c2oc2ccccc23)cc1 ; c1ccc(-c2ccc(N(c3ccc(-n4c5ccccc5c5ccccc54)cc3)c3cccc4c3oc3ccccc34)c3c2oc2ccccc23)cc1 ; c1ccc(-c2ccc(N(c3ccc4c5ccccc5n(-c5ccccc5)c4c3)c3cccc4sc5ccccc5c34)c3c2oc2ccccc23)cc1]

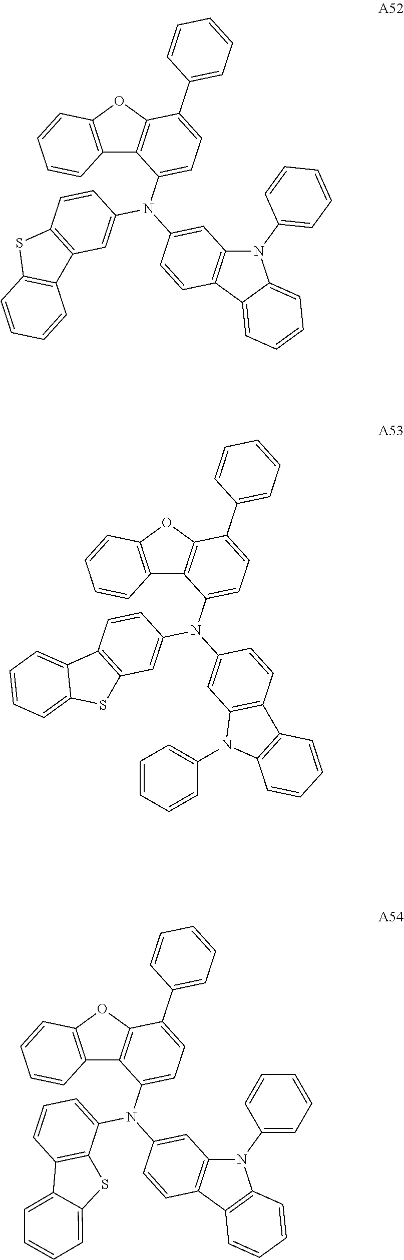 [SMILES: c1ccc(-c2ccc(N(c3ccc4sc5ccccc5c4c3)c3ccc4c5ccccc5n(-c5ccccc5)c4c3)c3c2oc2ccccc23)cc1 ; c1ccc(-c2ccc(N(c3ccc4c(c3)sc3ccccc34)c3ccc4c5ccccc5n(-c5ccccc5)c4c3)c3c2oc2ccccc23)cc1 ; c1ccc(-c2ccc(N(c3ccc4c5ccccc5n(-c5ccccc5)c4c3)c3cccc4c3sc3ccccc34)c3c2oc2ccccc23)cc1]

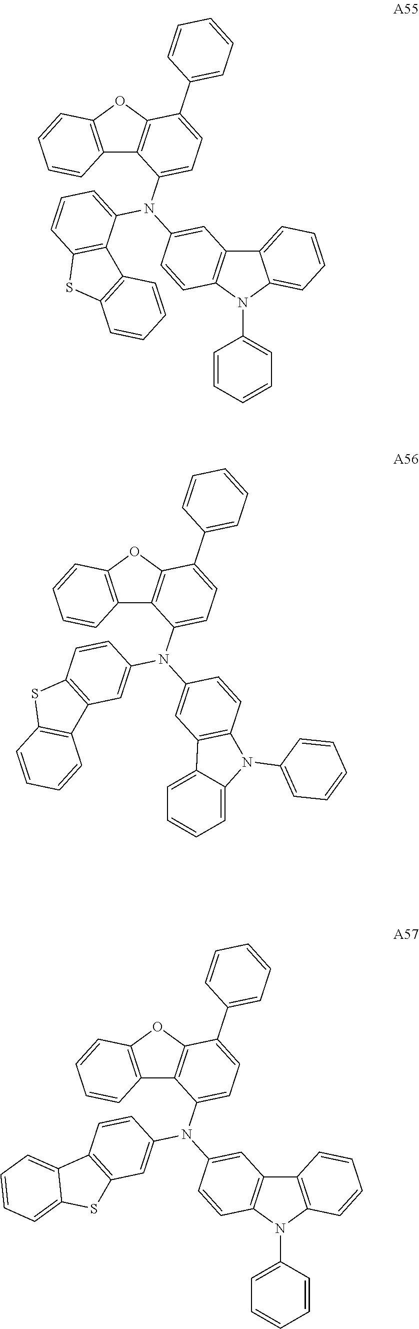 [SMILES: c1ccc(-c2ccc(N(c3ccc4c(c3)c3ccccc3n4-c3ccccc3)c3cccc4sc5ccccc5c34)c3c2oc2ccccc23)cc1 ; c1ccc(-c2ccc(N(c3ccc4sc5ccccc5c4c3)c3ccc4c(c3)c3ccccc3n4-c3ccccc3)c3c2oc2ccccc23)cc1 ; c1ccc(-c2ccc(N(c3ccc4c(c3)sc3ccccc34)c3ccc4c(c3)c3ccccc3n4-c3ccccc3)c3c2oc2ccccc23)cc1]

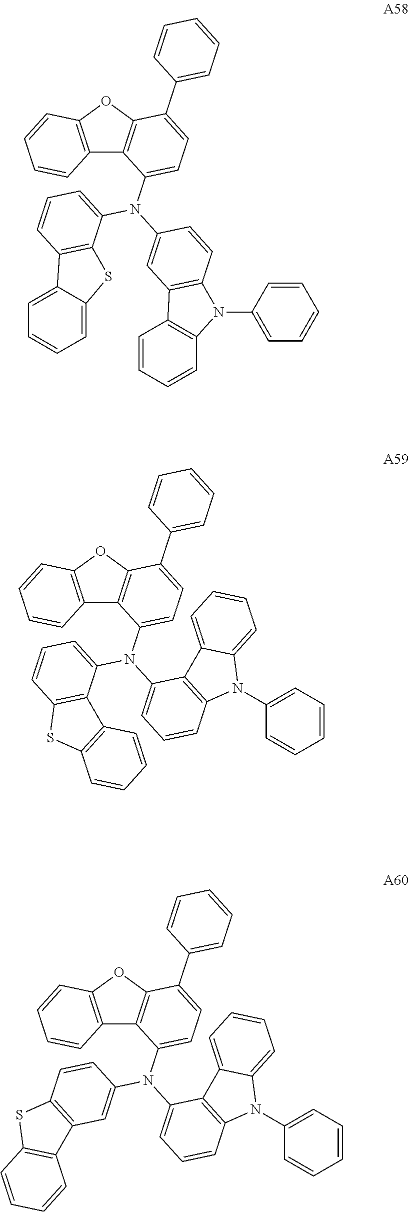 [SMILES: c1ccc(-c2ccc(N(c3ccc4c(c3)c3ccccc3n4-c3ccccc3)c3cccc4c3sc3ccccc34)c3c2oc2ccccc23)cc1 ; c1ccc(-c2ccc(N(c3cccc4sc5ccccc5c34)c3cccc4c3c3ccccc3n4-c3ccccc3)c3c2oc2ccccc23)cc1 ; c1ccc(-c2ccc(N(c3ccc4sc5ccccc5c4c3)c3cccc4c3c3ccccc3n4-c3ccccc3)c3c2oc2ccccc23)cc1]

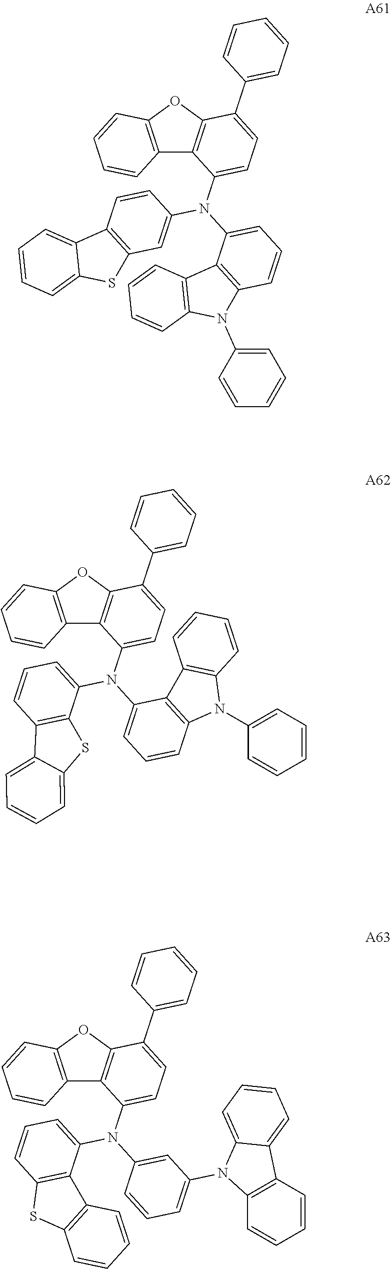 [SMILES: c1ccc(-c2ccc(N(c3ccc4c(c3)sc3ccccc34)c3cccc4c3c3ccccc3n4-c3ccccc3)c3c2oc2ccccc23)cc1 ; c1ccc(-c2ccc(N(c3cccc4c3sc3ccccc34)c3cccc4c3c3ccccc3n4-c3ccccc3)c3c2oc2ccccc23)cc1 ; c1ccc(-c2ccc(N(c3cccc(-n4c5ccccc5c5ccccc54)c3)c3cccc4sc5ccccc5c34)c3c2oc2ccccc23)cc1]

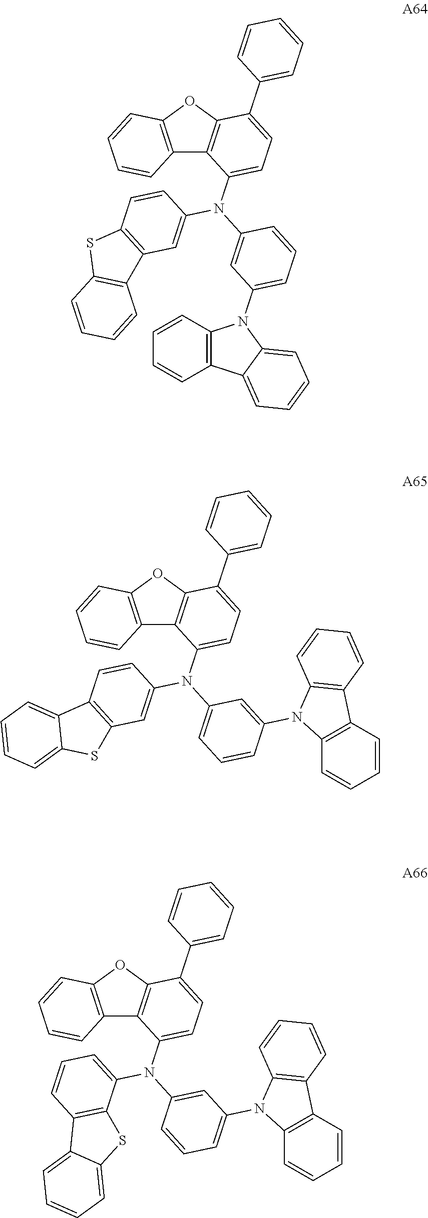 [SMILES: c1ccc(-c2ccc(N(c3cccc(-n4c5ccccc5c5ccccc54)c3)c3ccc4sc5ccccc5c4c3)c3c2oc2ccccc23)cc1 ; c1ccc(-c2ccc(N(c3cccc(-n4c5ccccc5c5ccccc54)c3)c3ccc4c(c3)sc3ccccc34)c3c2oc2ccccc23)cc1 ; c1ccc(-c2ccc(N(c3cccc(-n4c5ccccc5c5ccccc54)c3)c3cccc4c3sc3ccccc34)c3c2oc2ccccc23)cc1]

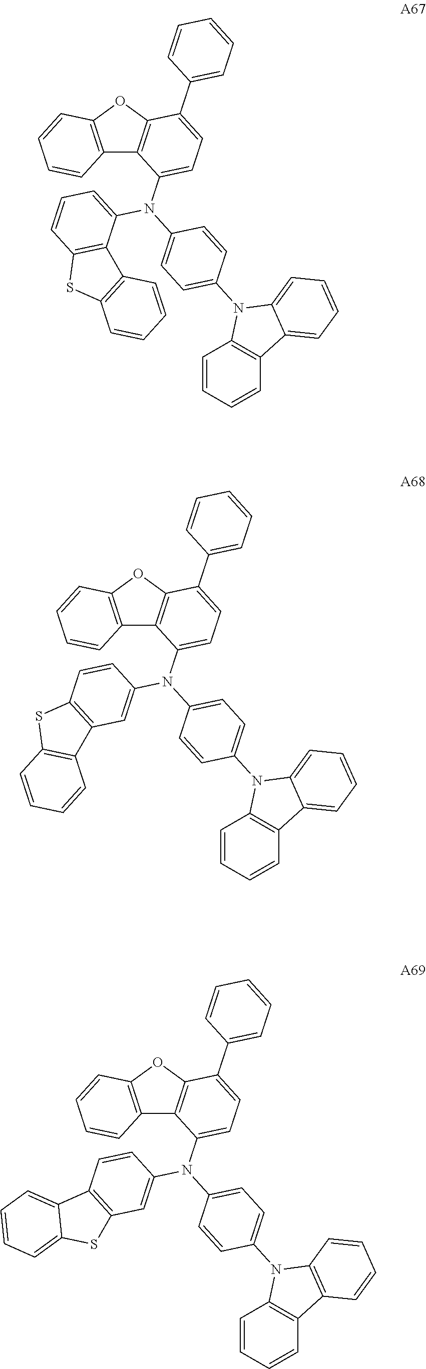 [SMILES: c1ccc(-c2ccc(N(c3ccc(-n4c5ccccc5c5ccccc54)cc3)c3cccc4sc5ccccc5c34)c3c2oc2ccccc23)cc1 ; c1ccc(-c2ccc(N(c3ccc(-n4c5ccccc5c5ccccc54)cc3)c3ccc4sc5ccccc5c4c3)c3c2oc2ccccc23)cc1 ; c1ccc(-c2ccc(N(c3ccc(-n4c5ccccc5c5ccccc54)cc3)c3ccc4c(c3)sc3ccccc34)c3c2oc2ccccc23)cc1]

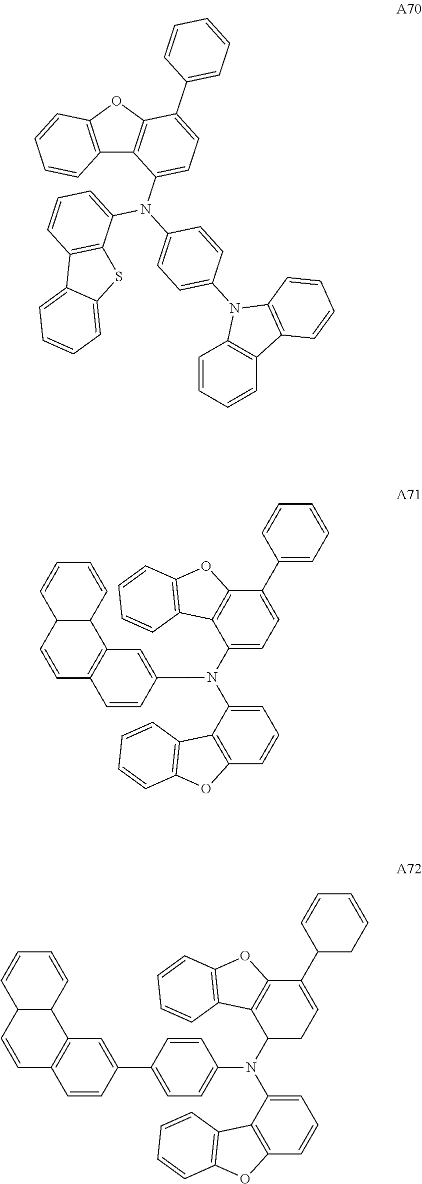 [SMILES: c1ccc(-c2ccc(N(c3ccc(-n4c5ccccc5c5ccccc54)cc3)c3cccc4c3sc3ccccc34)c3c2oc2ccccc23)cc1 ; C1=CC2C=Cc3ccc(N(c4cccc5oc6ccccc6c45)c4ccc(-c5ccccc5)c5oc6ccccc6c45)cc3C2C=C1 ; C1=CCC(C2=CCC(N(c3ccc(-c4ccc5c(c4)C4C=CC=CC4C=C5)cc3)c3cccc4oc5ccccc5c34)c3c2oc2ccccc32)C=C1]

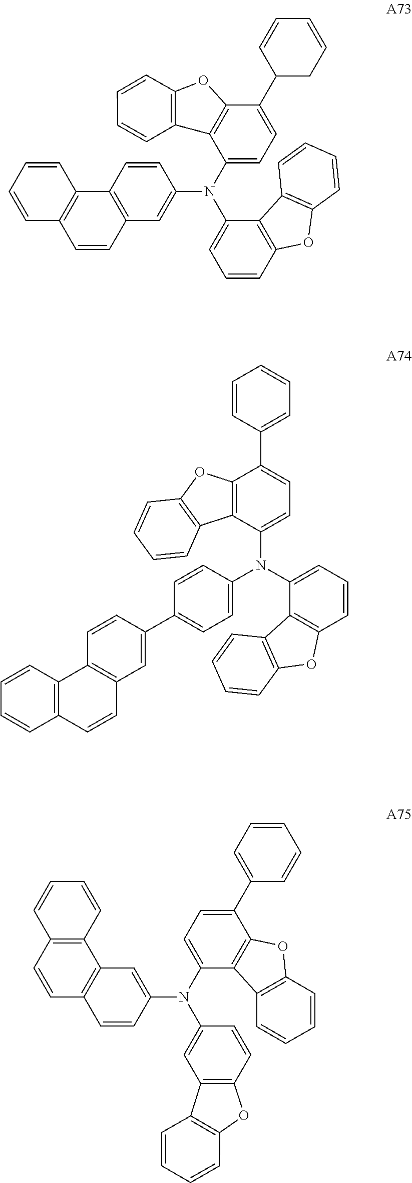 [SMILES: C1=CCC(c2ccc(N(c3ccc4c(ccc5ccccc54)c3)c3cccc4oc5ccccc5c34)c3c2oc2ccccc23)C=C1 ; c1ccc(-c2ccc(N(c3ccc(-c4ccc5c(ccc6ccccc65)c4)cc3)c3cccc4oc5ccccc5c34)c3c2oc2ccccc23)cc1 ; c1ccc(-c2ccc(N(c3ccc4ccc5ccccc5c4c3)c3ccc4oc5ccccc5c4c3)c3c2oc2ccccc23)cc1]

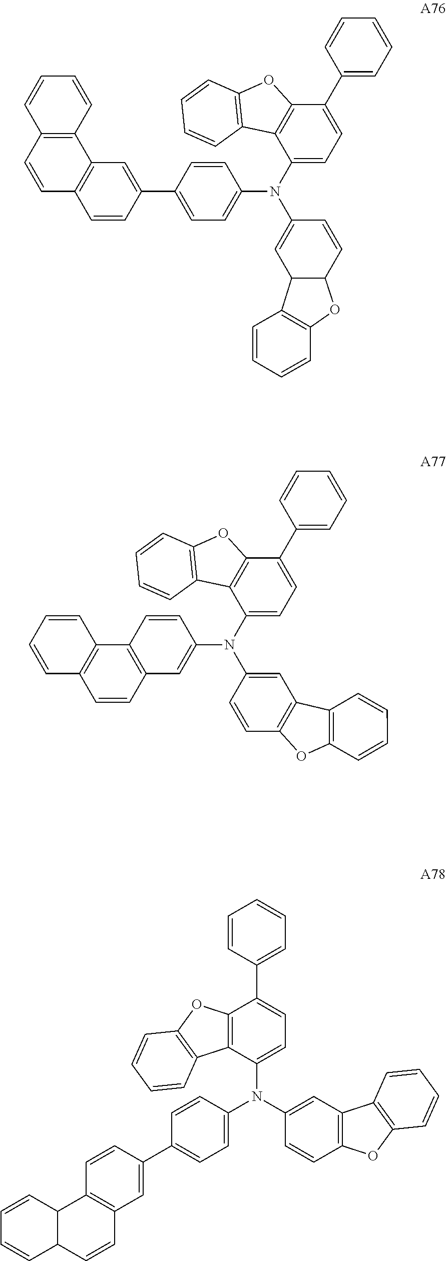 [SMILES: C1=CC2Oc3ccccc3C2C=C1N(c1ccc(-c2ccc3ccc4ccccc4c3c2)cc1)c1ccc(-c2ccccc2)c2oc3ccccc3c12 ; c1ccc(-c2ccc(N(c3ccc4c(ccc5ccccc54)c3)c3ccc4oc5ccccc5c4c3)c3c2oc2ccccc23)cc1 ; C1=CC2C=Cc3cc(-c4ccc(N(c5ccc6oc7ccccc7c6c5)c5ccc(-c6ccccc6)c6oc7ccccc7c56)cc4)ccc3C2C=C1]

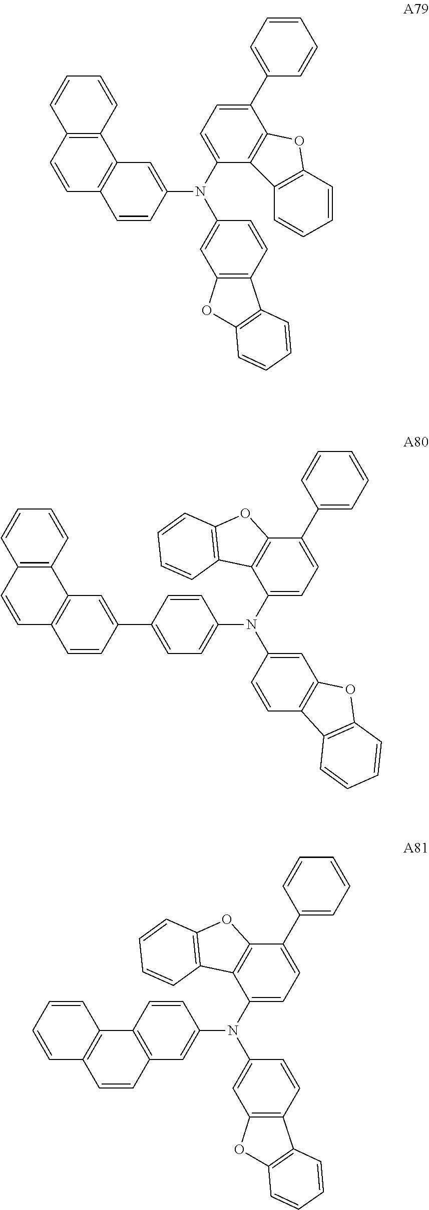 [SMILES: c1ccc(-c2ccc(N(c3ccc4c(c3)oc3ccccc34)c3ccc4ccc5ccccc5c4c3)c3c2oc2ccccc23)cc1 ; c1ccc(-c2ccc(N(c3ccc(-c4ccc5ccc6ccccc6c5c4)cc3)c3ccc4c(c3)oc3ccccc34)c3c2oc2ccccc23)cc1 ; c1ccc(-c2ccc(N(c3ccc4c(ccc5ccccc54)c3)c3ccc4c(c3)oc3ccccc34)c3c2oc2ccccc23)cc1]

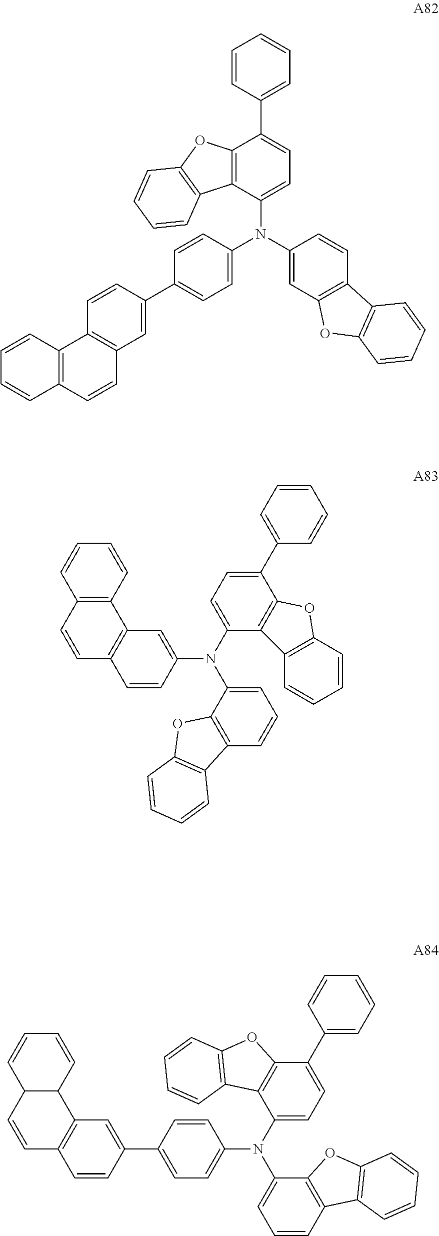 [SMILES: c1ccc(-c2ccc(N(c3ccc(-c4ccc5c(ccc6ccccc65)c4)cc3)c3ccc4c(c3)oc3ccccc34)c3c2oc2ccccc23)cc1 ; c1ccc(-c2ccc(N(c3ccc4ccc5ccccc5c4c3)c3cccc4c3oc3ccccc34)c3c2oc2ccccc23)cc1 ; C1=CC2C=Cc3ccc(-c4ccc(N(c5cccc6c5oc5ccccc56)c5ccc(-c6ccccc6)c6oc7ccccc7c56)cc4)cc3C2C=C1]

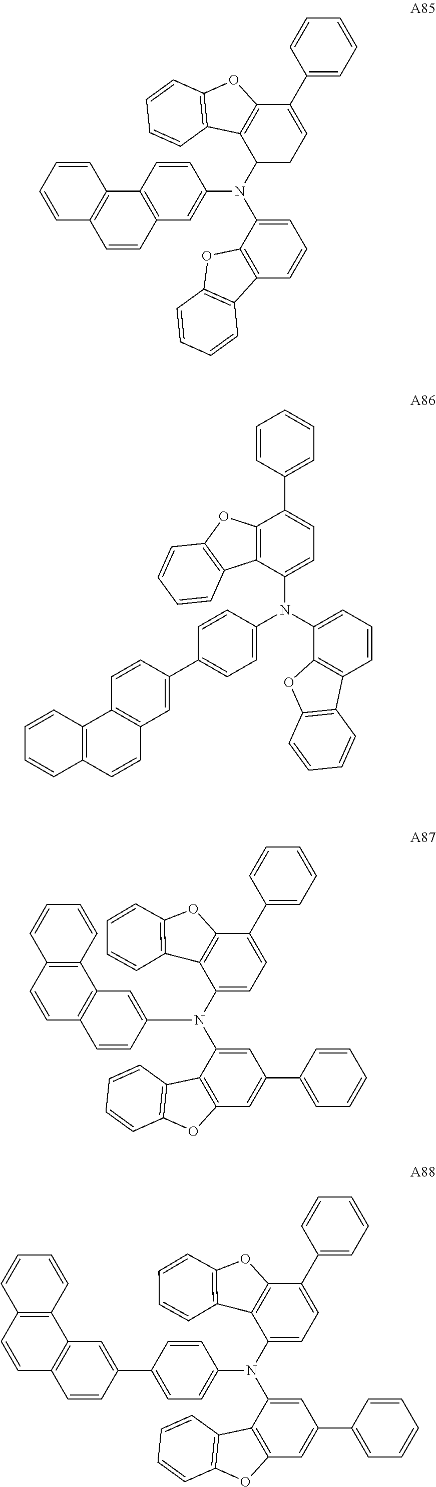 [SMILES: C1=C(c2ccccc2)c2oc3ccccc3c2C(N(c2ccc3c(ccc4ccccc43)c2)c2cccc3c2oc2ccccc23)C1 ; c1ccc(-c2ccc(N(c3ccc(-c4ccc5c(ccc6ccccc65)c4)cc3)c3cccc4c3oc3ccccc34)c3c2oc2ccccc23)cc1 ; c1ccc(-c2cc(N(c3ccc4ccc5ccccc5c4c3)c3ccc(-c4ccccc4)c4oc5ccccc5c34)c3c(c2)oc2ccccc23)cc1 ; c1ccc(-c2cc(N(c3ccc(-c4ccc5ccc6ccccc6c5c4)cc3)c3ccc(-c4ccccc4)c4oc5ccccc5c34)c3c(c2)oc2ccccc23)cc1]

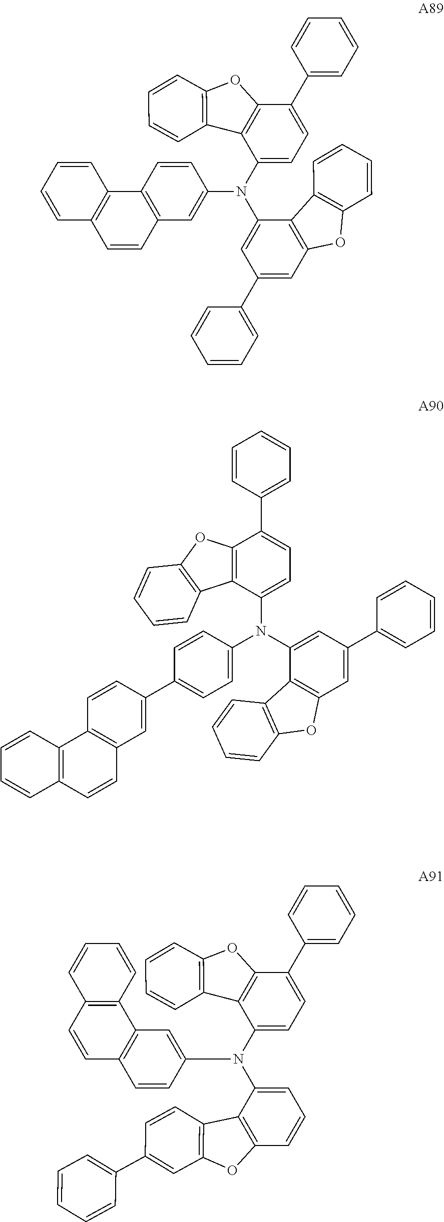 [SMILES: c1ccc(-c2cc(N(c3ccc4c(ccc5ccccc54)c3)c3ccc(-c4ccccc4)c4oc5ccccc5c34)c3c(c2)oc2ccccc23)cc1 ; c1ccc(-c2cc(N(c3ccc(-c4ccc5c(ccc6ccccc65)c4)cc3)c3ccc(-c4ccccc4)c4oc5ccccc5c34)c3c(c2)oc2ccccc23)cc1 ; c1ccc(-c2ccc3c(c2)oc2cccc(N(c4ccc5ccc6ccccc6c5c4)c4ccc(-c5ccccc5)c5oc6ccccc6c45)c23)cc1]

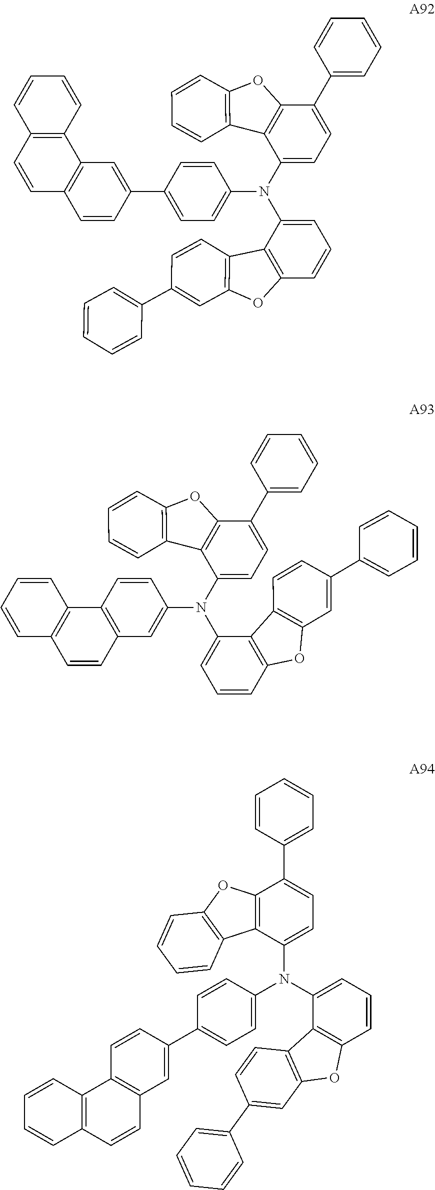 [SMILES: c1ccc(-c2ccc3c(c2)oc2cccc(N(c4ccc(-c5ccc6ccc7ccccc7c6c5)cc4)c4ccc(-c5ccccc5)c5oc6ccccc6c45)c23)cc1 ; c1ccc(-c2ccc3c(c2)oc2cccc(N(c4ccc5c(ccc6ccccc65)c4)c4ccc(-c5ccccc5)c5oc6ccccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5oc6ccccc6c45)c23)cc1]

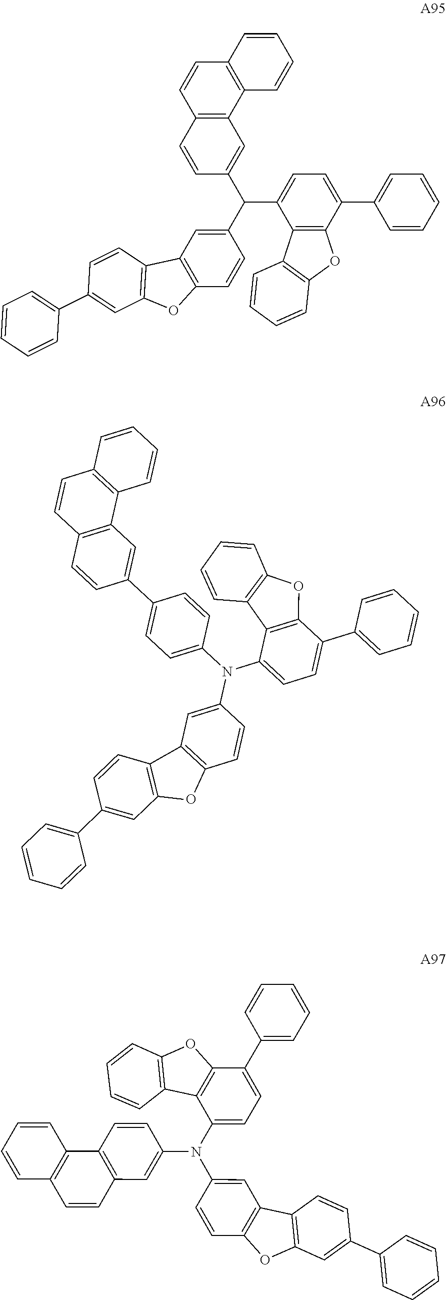 [SMILES: c1ccc(-c2ccc3c(c2)oc2ccc(C(c4ccc5ccc6ccccc6c5c4)c4ccc(-c5ccccc5)c5oc6ccccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5oc6ccccc6c45)cc23)cc1 ; c1ccc(-c2ccc3c(c2)oc2ccc(N(c4ccc5c(ccc6ccccc65)c4)c4ccc(-c5ccccc5)c5oc6ccccc6c45)cc23)cc1]

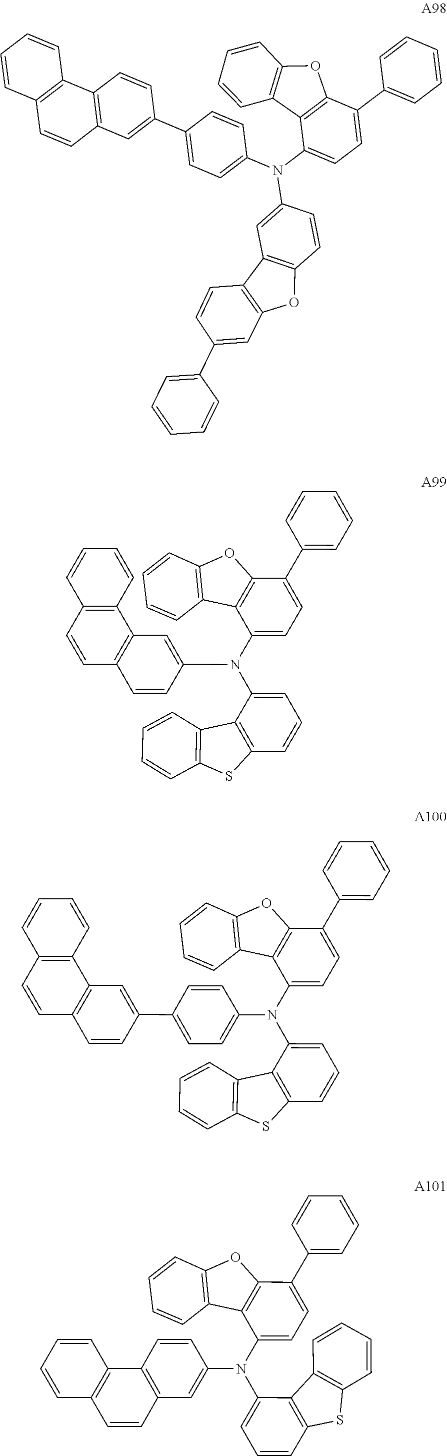 [SMILES: c1ccc(-c2ccc3c(c2)oc2ccc(N(c4ccc(-c5ccc6c(ccc7ccccc76)c5)cc4)c4ccc(-c5ccccc5)c5oc6ccccc6c45)cc23)cc1 ; c1ccc(-c2ccc(N(c3ccc4ccc5ccccc5c4c3)c3cccc4sc5ccccc5c34)c3c2oc2ccccc23)cc1 ; c1ccc(-c2ccc(N(c3ccc(-c4ccc5ccc6ccccc6c5c4)cc3)c3cccc4sc5ccccc5c34)c3c2oc2ccccc23)cc1 ; c1ccc(-c2ccc(N(c3ccc4c(ccc5ccccc54)c3)c3cccc4sc5ccccc5c34)c3c2oc2ccccc23)cc1]

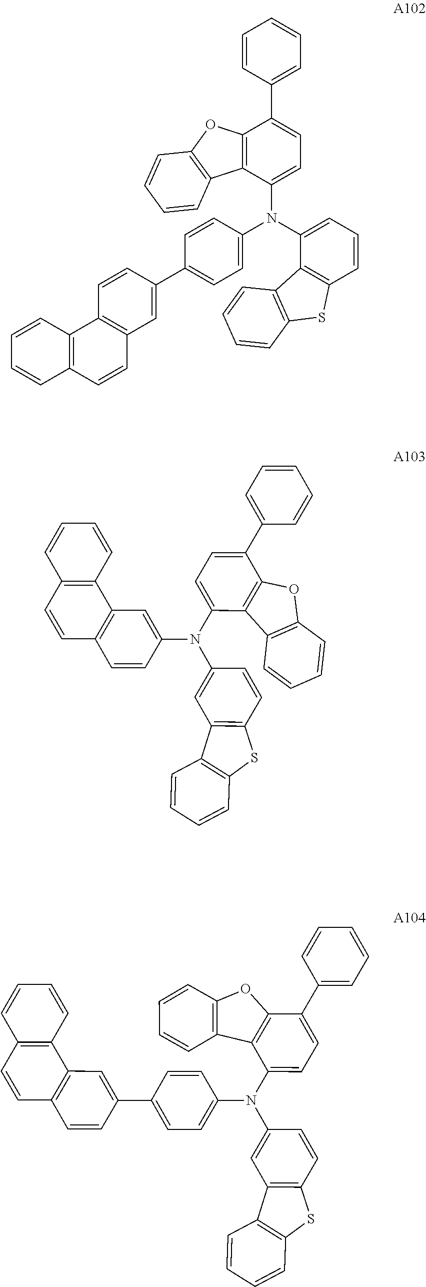 [SMILES: c1ccc(-c2ccc(N(c3ccc(-c4ccc5c(ccc6ccccc65)c4)cc3)c3cccc4sc5ccccc5c34)c3c2oc2ccccc23)cc1 ; c1ccc(-c2ccc(N(c3ccc4ccc5ccccc5c4c3)c3ccc4sc5ccccc5c4c3)c3c2oc2ccccc23)cc1 ; c1ccc(-c2ccc(N(c3ccc(-c4ccc5ccc6ccccc6c5c4)cc3)c3ccc4sc5ccccc5c4c3)c3c2oc2ccccc23)cc1]

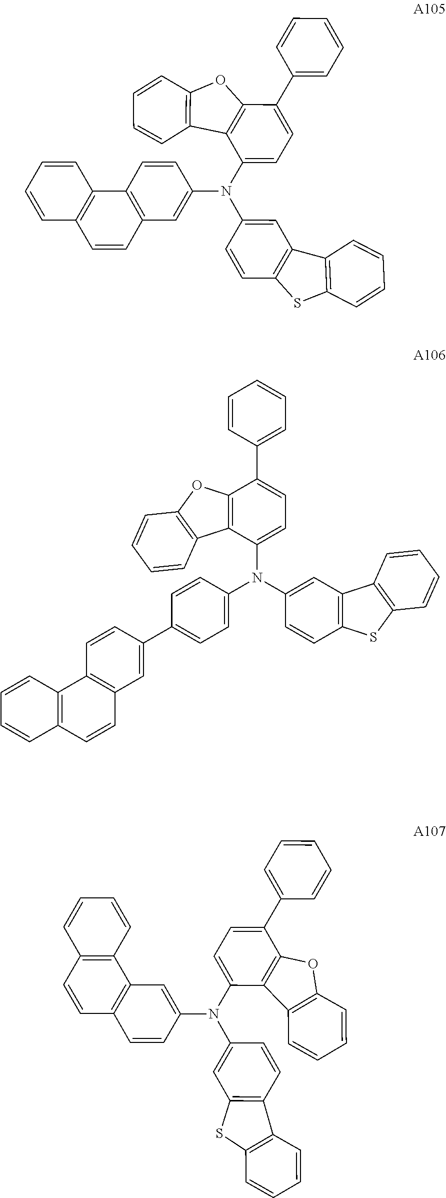 [SMILES: c1ccc(-c2ccc(N(c3ccc4c(ccc5ccccc54)c3)c3ccc4sc5ccccc5c4c3)c3c2oc2ccccc23)cc1 ; c1ccc(-c2ccc(N(c3ccc(-c4ccc5c(ccc6ccccc65)c4)cc3)c3ccc4sc5ccccc5c4c3)c3c2oc2ccccc23)cc1 ; c1ccc(-c2ccc(N(c3ccc4c(c3)sc3ccccc34)c3ccc4ccc5ccccc5c4c3)c3c2oc2ccccc23)cc1]

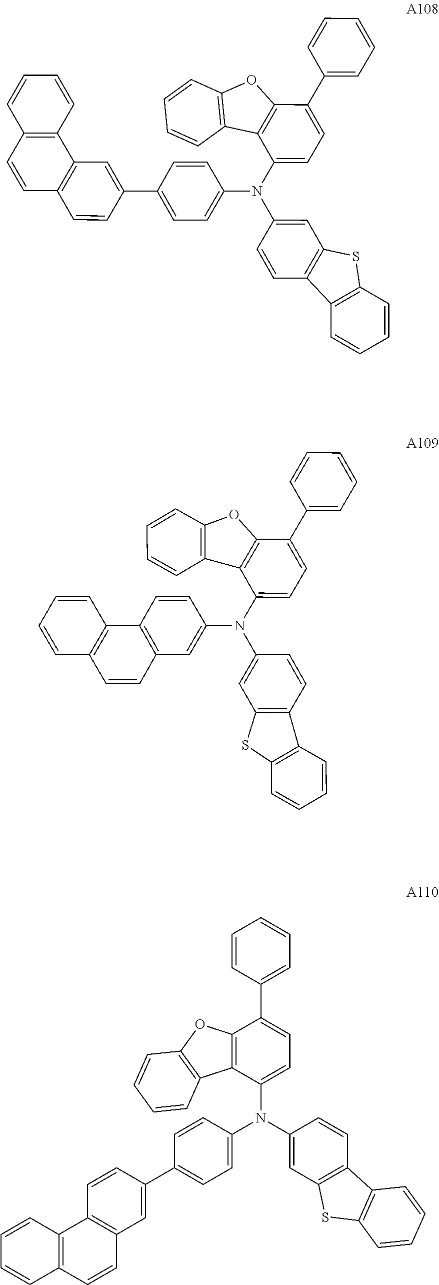 [SMILES: c1ccc(-c2ccc(N(c3ccc(-c4ccc5ccc6ccccc6c5c4)cc3)c3ccc4c(c3)sc3ccccc34)c3c2oc2ccccc23)cc1 ; c1ccc(-c2ccc(N(c3ccc4c(ccc5ccccc54)c3)c3ccc4c(c3)sc3ccccc34)c3c2oc2ccccc23)cc1 ; c1ccc(-c2ccc(N(c3ccc(-c4ccc5c(ccc6ccccc65)c4)cc3)c3ccc4c(c3)sc3ccccc34)c3c2oc2ccccc23)cc1]

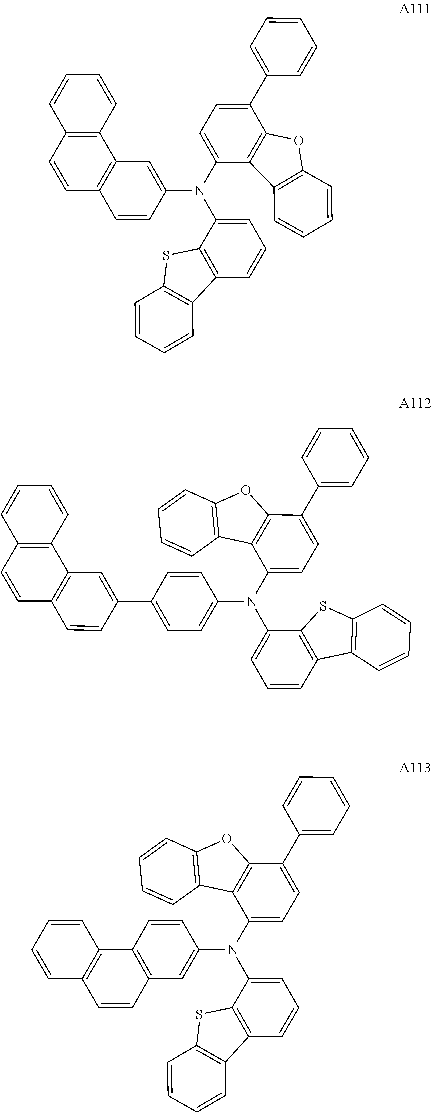 [SMILES: c1ccc(-c2ccc(N(c3ccc4ccc5ccccc5c4c3)c3cccc4c3sc3ccccc34)c3c2oc2ccccc23)cc1 ; c1ccc(-c2ccc(N(c3ccc(-c4ccc5ccc6ccccc6c5c4)cc3)c3cccc4c3sc3ccccc34)c3c2oc2ccccc23)cc1 ; c1ccc(-c2ccc(N(c3ccc4c(ccc5ccccc54)c3)c3cccc4c3sc3ccccc34)c3c2oc2ccccc23)cc1]

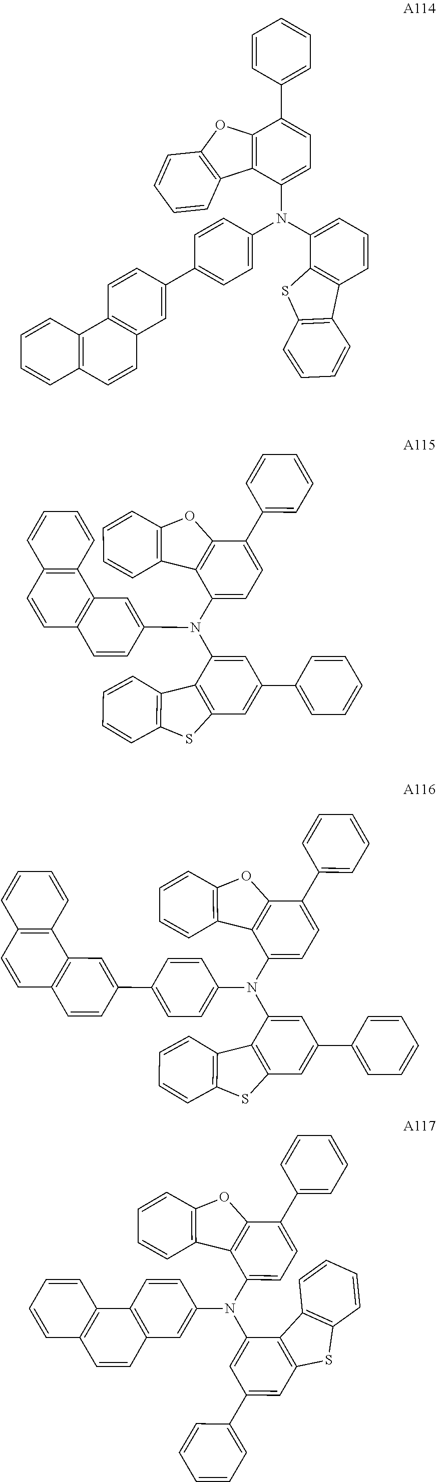 [SMILES: c1ccc(-c2ccc(N(c3ccc(-c4ccc5c(ccc6ccccc65)c4)cc3)c3cccc4c3sc3ccccc34)c3c2oc2ccccc23)cc1 ; c1ccc(-c2cc(N(c3ccc4ccc5ccccc5c4c3)c3ccc(-c4ccccc4)c4oc5ccccc5c34)c3c(c2)sc2ccccc23)cc1 ; c1ccc(-c2cc(N(c3ccc(-c4ccc5ccc6ccccc6c5c4)cc3)c3ccc(-c4ccccc4)c4oc5ccccc5c34)c3c(c2)sc2ccccc23)cc1 ; c1ccc(-c2cc(N(c3ccc4c(ccc5ccccc54)c3)c3ccc(-c4ccccc4)c4oc5ccccc5c34)c3c(c2)sc2ccccc23)cc1]

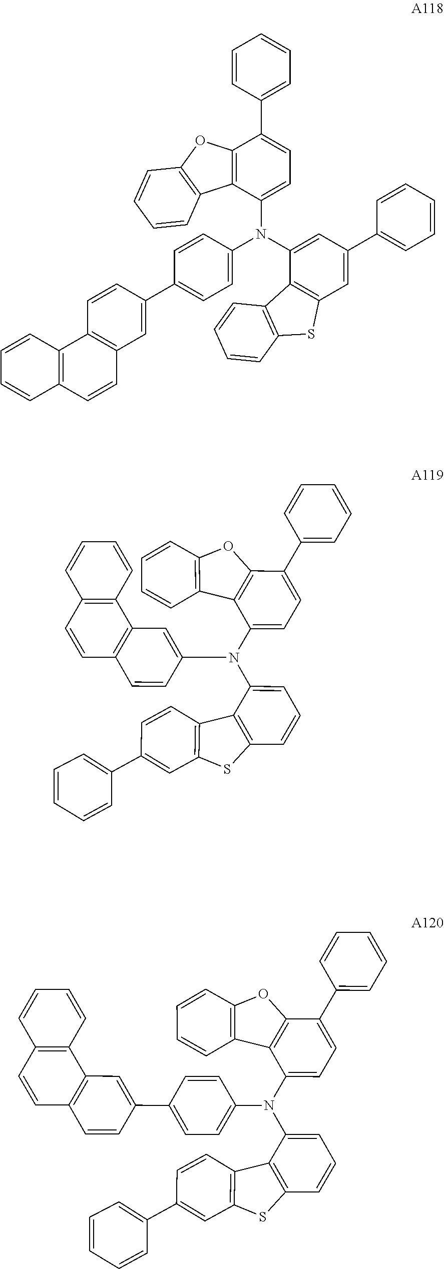 [SMILES: c1ccc(-c2cc(N(c3ccc(-c4ccc5c(ccc6ccccc65)c4)cc3)c3ccc(-c4ccccc4)c4oc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5oc6ccccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5oc6ccccc6c45)c23)cc1]

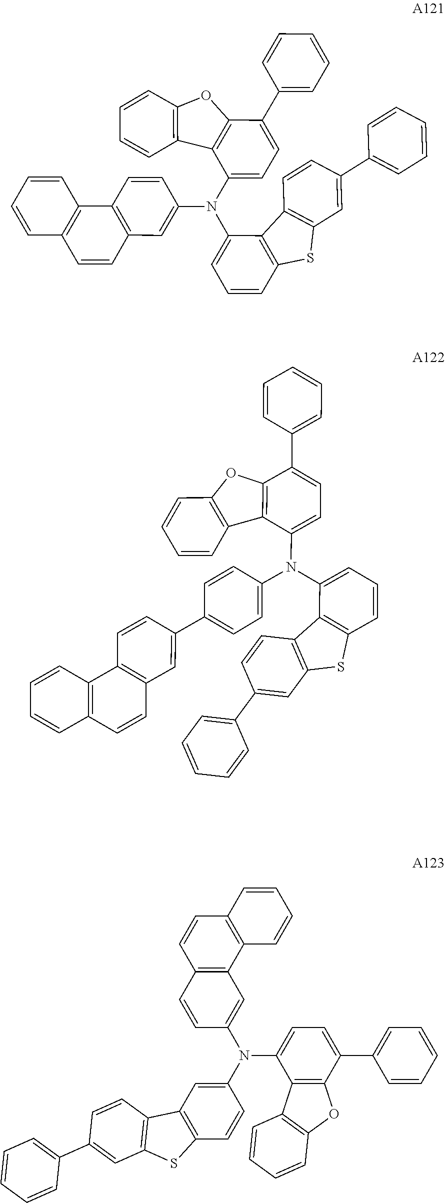 [SMILES: c1ccc(-c2ccc3c(c2)sc2cccc(N(c4ccc5c(ccc6ccccc65)c4)c4ccc(-c5ccccc5)c5oc6ccccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5oc6ccccc6c45)c23)cc1 ; c1ccc(-c2ccc3c(c2)sc2ccc(N(c4ccc5ccc6ccccc6c5c4)c4ccc(-c5ccccc5)c5oc6ccccc6c45)cc23)cc1]

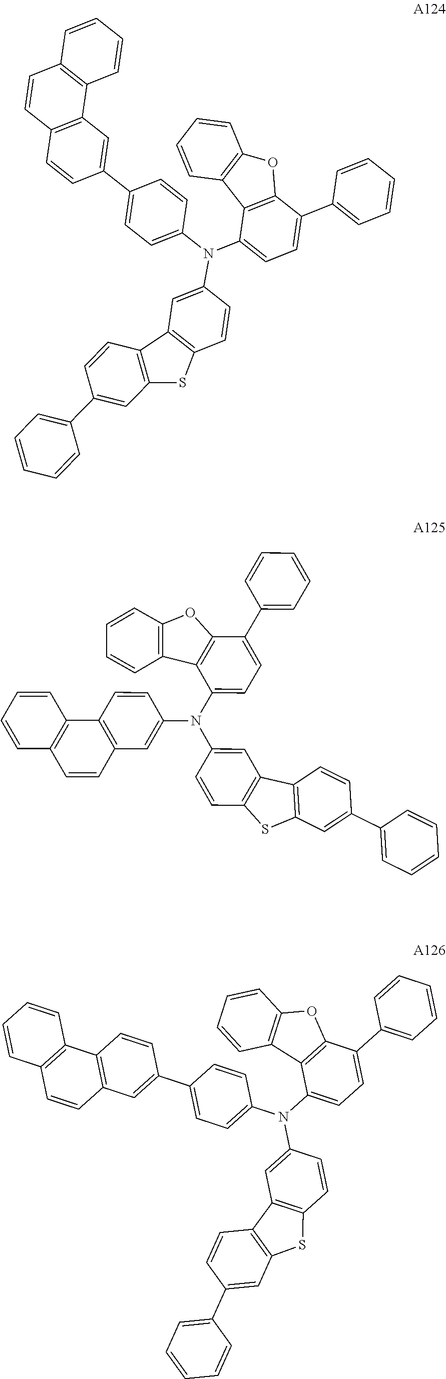 [SMILES: c1ccc(-c2ccc3c(c2)sc2ccc(N(c4ccc(-c5ccc6ccc7ccccc7c6c5)cc4)c4ccc(-c5ccccc5)c5oc6ccccc6c45)cc23)cc1 ; c1ccc(-c2ccc3c(c2)sc2ccc(N(c4ccc5c(ccc6ccccc65)c4)c4ccc(-c5ccccc5)c5oc6ccccc6c45)cc23)cc1 ; c1ccc(-c2ccc3c(c2)sc2ccc(N(c4ccc(-c5ccc6c(ccc7ccccc76)c5)cc4)c4ccc(-c5ccccc5)c5oc6ccccc6c45)cc23)cc1]

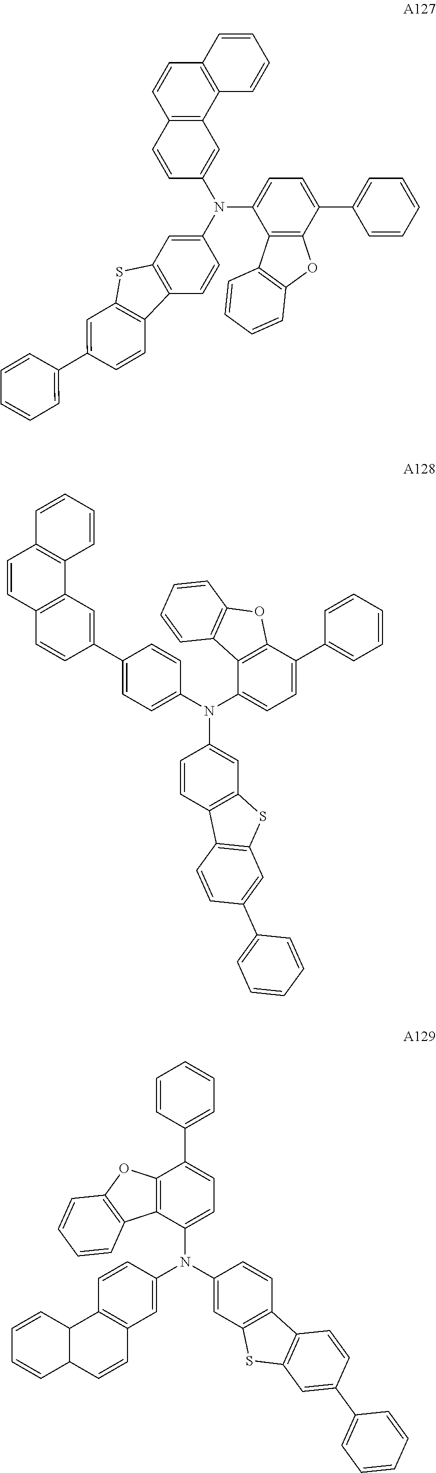 [SMILES: c1ccc(-c2ccc3c(c2)sc2cc(N(c4ccc5ccc6ccccc6c5c4)c4ccc(-c5ccccc5)c5oc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5oc6ccccc6c45)ccc23)cc1 ; C1=CC2C=Cc3cc(N(c4ccc5c(c4)sc4cc(-c6ccccc6)ccc45)c4ccc(-c5ccccc5)c5oc6ccccc6c45)ccc3C2C=C1]

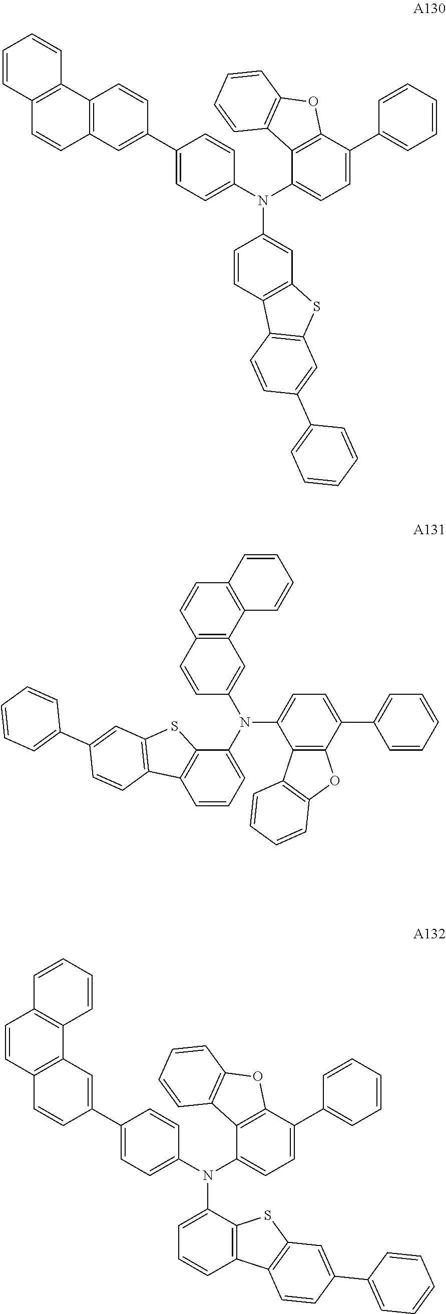 [SMILES: c1ccc(-c2ccc3c(c2)sc2cc(N(c4ccc(-c5ccc6c(ccc7ccccc76)c5)cc4)c4ccc(-c5ccccc5)c5oc6ccccc6c45)ccc23)cc1 ; c1ccc(-c2ccc3c(c2)sc2c(N(c4ccc5ccc6ccccc6c5c4)c4ccc(-c5ccccc5)c5oc6ccccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5oc6ccccc6c45)cccc23)cc1]

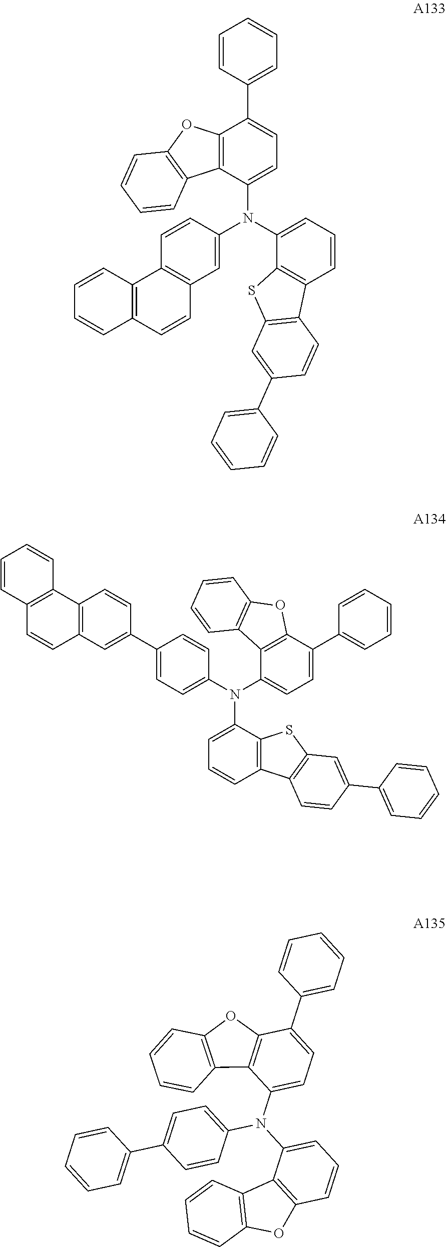 [SMILES: c1ccc(-c2ccc3c(c2)sc2c(N(c4ccc5c(ccc6ccccc65)c4)c4ccc(-c5ccccc5)c5oc6ccccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5oc6ccccc6c45)cccc23)cc1 ; c1ccc(-c2ccc(N(c3cccc4oc5ccccc5c34)c3ccc(-c4ccccc4)c4oc5ccccc5c34)cc2)cc1]

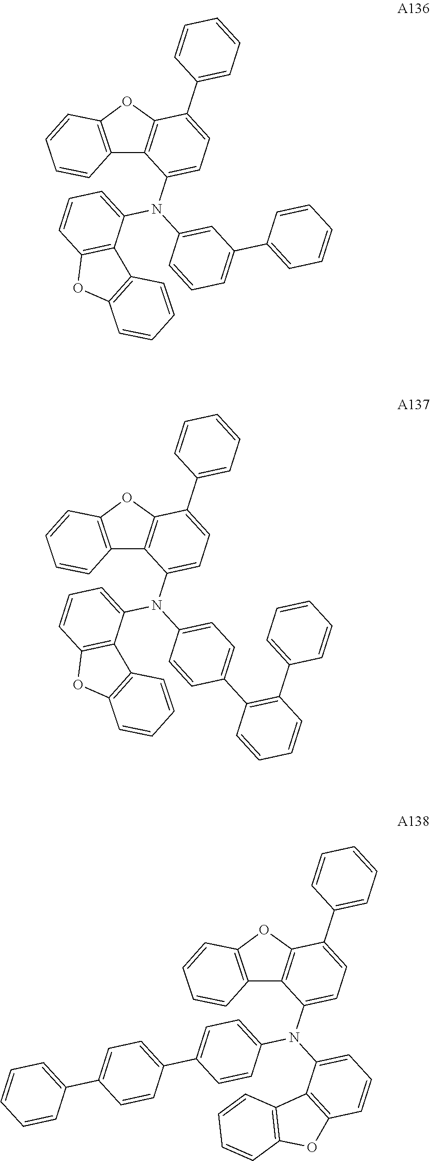 [SMILES: c1ccc(-c2cccc(N(c3cccc4oc5ccccc5c34)c3ccc(-c4ccccc4)c4oc5ccccc5c34)c2)cc1 ; c1ccc(-c2ccccc2-c2ccc(N(c3cccc4oc5ccccc5c34)c3ccc(-c4ccccc4)c4oc5ccccc5c34)cc2)cc1 ; c1ccc(-c2ccc(-c3ccc(N(c4cccc5oc6ccccc6c45)c4ccc(-c5ccccc5)c5oc6ccccc6c45)cc3)cc2)cc1]

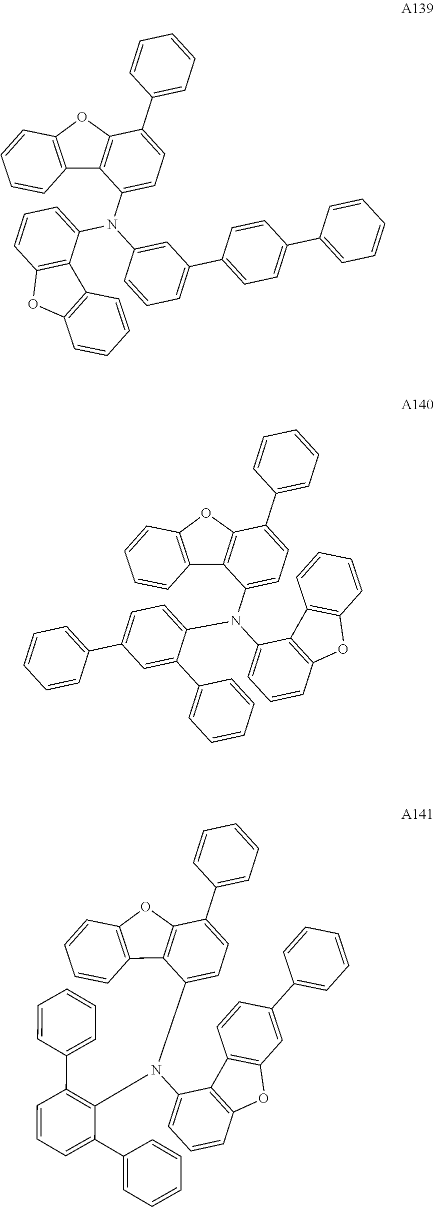 [SMILES: c1ccc(-c2ccc(-c3cccc(N(c4cccc5oc6ccccc6c45)c4ccc(-c5ccccc5)c5oc6ccccc6c45)c3)cc2)cc1 ; c1ccc(-c2ccc(N(c3cccc4oc5ccccc5c34)c3ccc(-c4ccccc4)c4oc5ccccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5oc6ccccc6c45)c23)cc1]

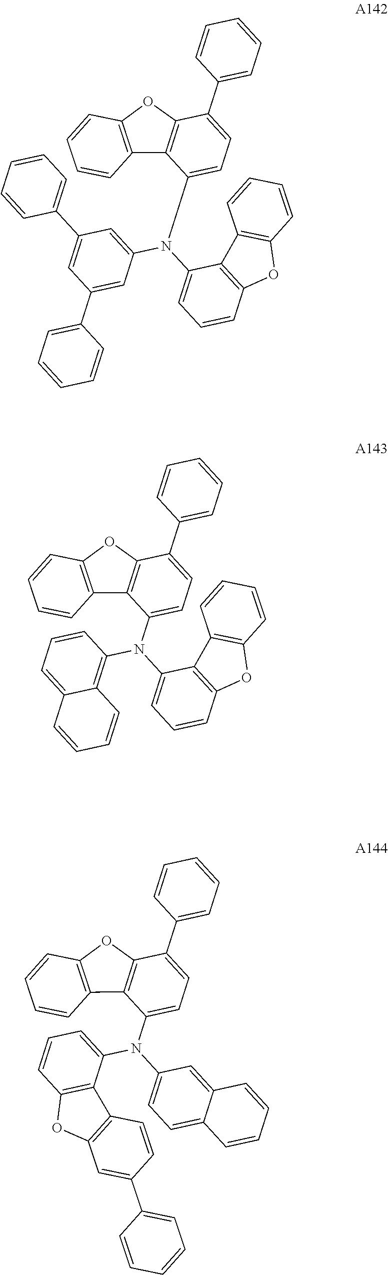 [SMILES: c1ccc(-c2cc(-c3ccccc3)cc(N(c3cccc4oc5ccccc5c34)c3ccc(-c4ccccc4)c4oc5ccccc5c34)c2)cc1 ; c1ccc(-c2ccc(N(c3cccc4ccccc34)c3cccc4oc5ccccc5c34)c3c2oc2ccccc23)cc1 ; c1ccc(-c2ccc3c(c2)oc2cccc(N(c4ccc5ccccc5c4)c4ccc(-c5ccccc5)c5oc6ccccc6c45)c23)cc1]

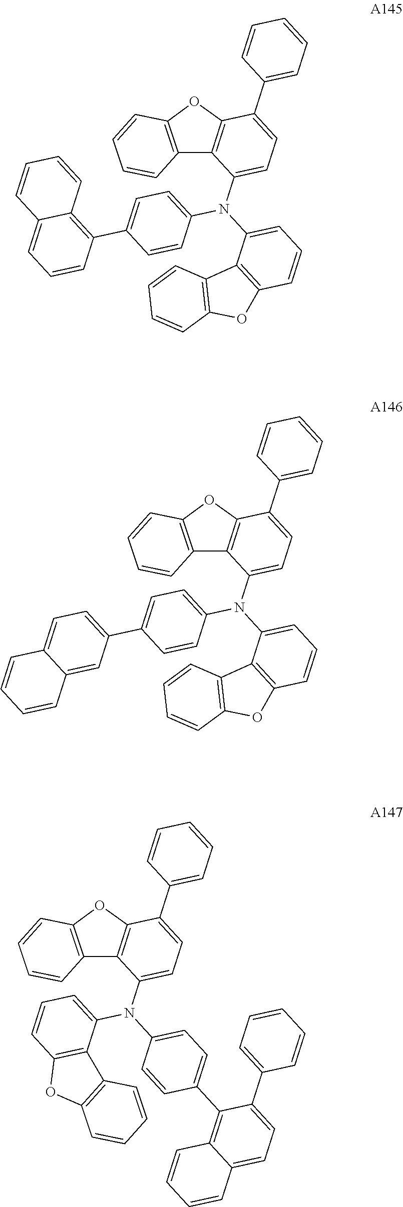 [SMILES: c1ccc(-c2ccc(N(c3ccc(-c4cccc5ccccc45)cc3)c3cccc4oc5ccccc5c34)c3c2oc2ccccc23)cc1 ; c1ccc(-c2ccc(N(c3ccc(-c4ccc5ccccc5c4)cc3)c3cccc4oc5ccccc5c34)c3c2oc2ccccc23)cc1 ; c1ccc(-c2ccc3ccccc3c2-c2ccc(N(c3cccc4oc5ccccc5c34)c3ccc(-c4ccccc4)c4oc5ccccc5c34)cc2)cc1]

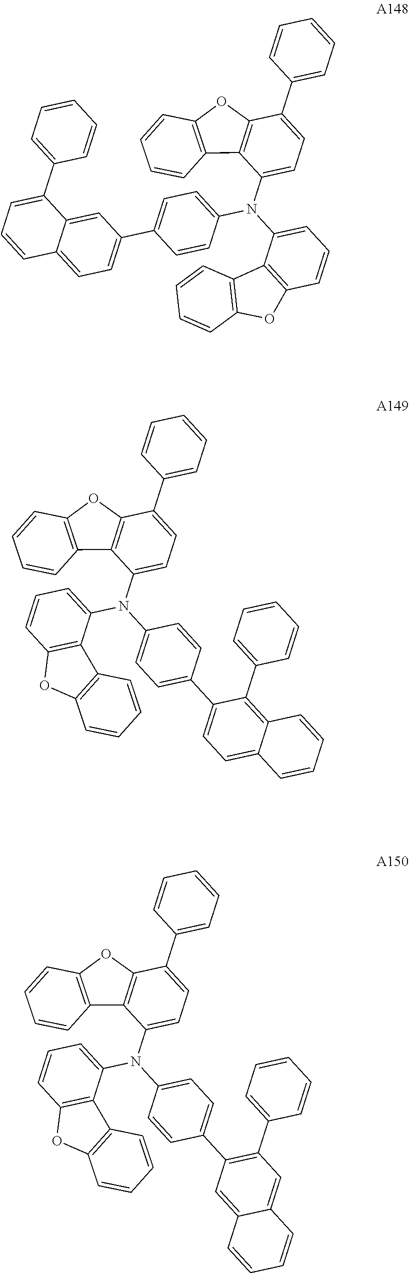 [SMILES: c1ccc(-c2cccc3ccc(-c4ccc(N(c5cccc6oc7ccccc7c56)c5ccc(-c6ccccc6)c6oc7ccccc7c56)cc4)cc23)cc1 ; c1ccc(-c2c(-c3ccc(N(c4cccc5oc6ccccc6c45)c4ccc(-c5ccccc5)c5oc6ccccc6c45)cc3)ccc3ccccc23)cc1 ; c1ccc(-c2cc3ccccc3cc2-c2ccc(N(c3cccc4oc5ccccc5c34)c3ccc(-c4ccccc4)c4oc5ccccc5c34)cc2)cc1]

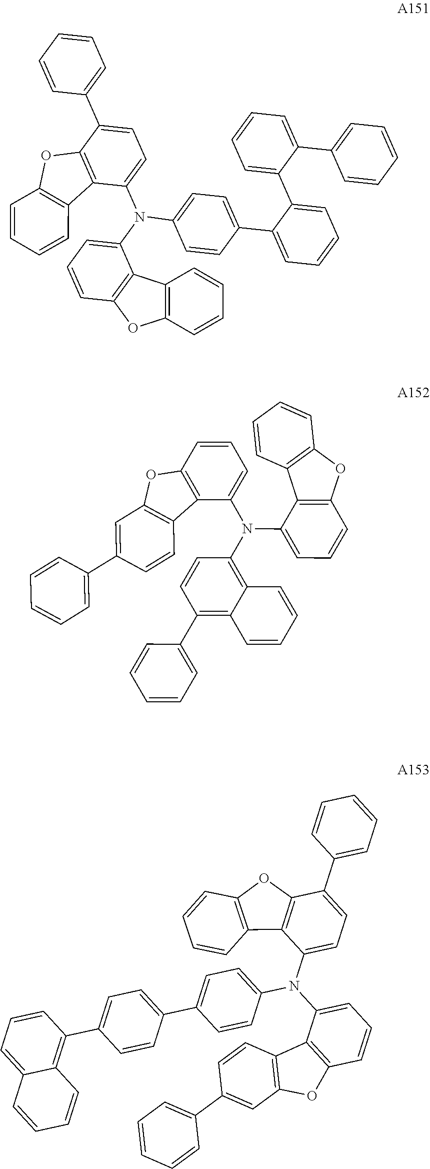 [SMILES: c1ccc(-c2ccccc2-c2ccccc2-c2ccc(N(c3cccc4oc5ccccc5c34)c3ccc(-c4ccccc4)c4oc5ccccc5c34)cc2)cc1 ; c1ccc(-c2ccc3c(c2)oc2cccc(N(c4ccc(-c5ccccc5)c5ccccc45)c4cccc5oc6ccccc6c45)c23)cc1 ; c1ccc(-c2ccc3c(c2)oc2cccc(N(c4ccc(-c5ccc(-c6cccc7ccccc67)cc5)cc4)c4ccc(-c5ccccc5)c5oc6ccccc6c45)c23)cc1]

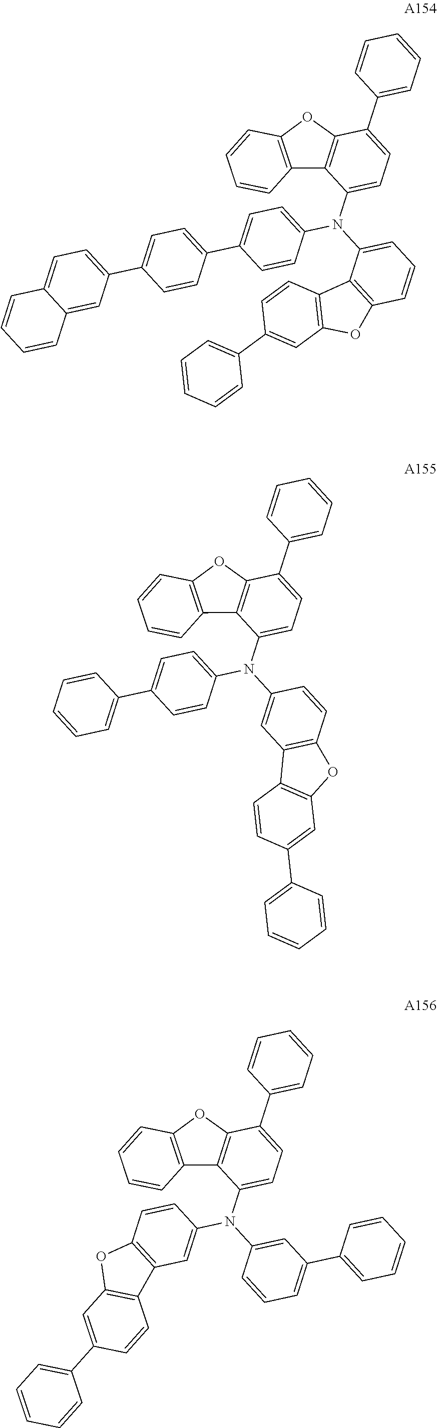 [SMILES: c1ccc(-c2ccc3c(c2)oc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4oc5cc(-c6ccccc6)ccc5c4c3)c3ccc(-c4ccccc4)c4oc5ccccc5c34)cc2)cc1 ; c1ccc(-c2cccc(N(c3ccc4oc5cc(-c6ccccc6)ccc5c4c3)c3ccc(-c4ccccc4)c4oc5ccccc5c34)c2)cc1]

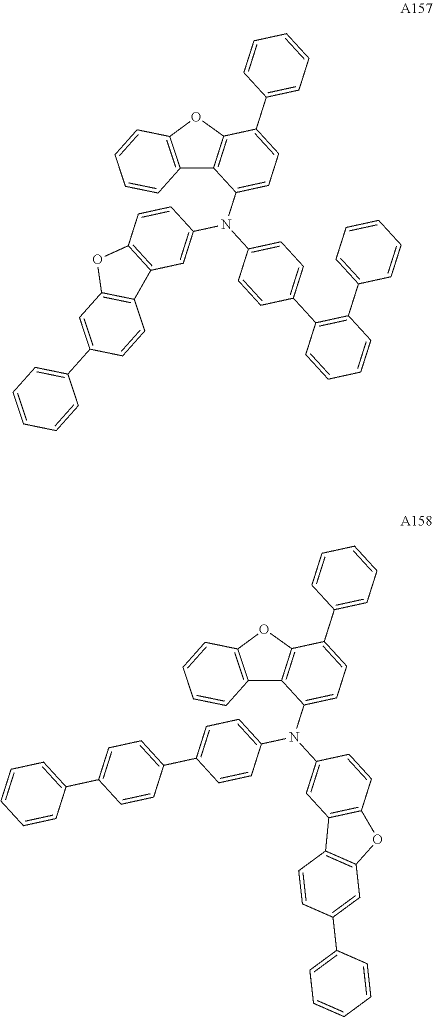 [SMILES: c1ccc(-c2ccc3c(c2)oc2ccc(N(c4ccc(-c5ccccc5-c5ccccc5)cc4)c4ccc(-c5ccccc5)c5oc6ccccc6c45)cc23)cc1 ; c1ccc(-c2ccc(-c3ccc(N(c4ccc5oc6cc(-c7ccccc7)ccc6c5c4)c4ccc(-c5ccccc5)c5oc6ccccc6c45)cc3)cc2)cc1]

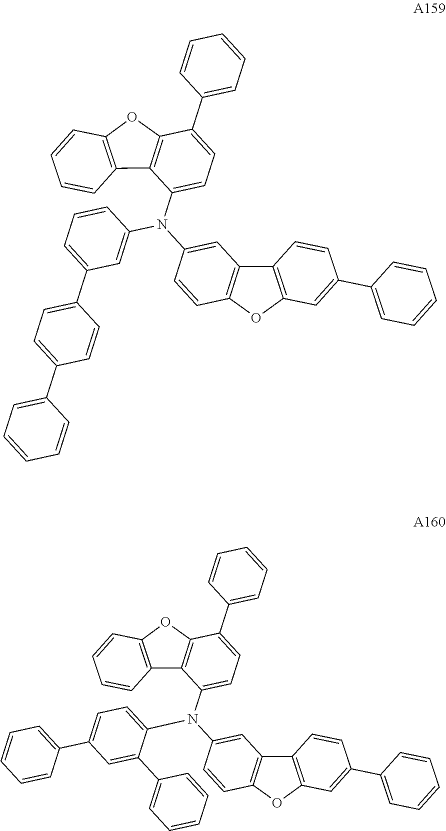 [SMILES: c1ccc(-c2ccc(-c3cccc(N(c4ccc5oc6cc(-c7ccccc7)ccc6c5c4)c4ccc(-c5ccccc5)c5oc6ccccc6c45)c3)cc2)cc1 ; c1ccc(-c2ccc(N(c3ccc4oc5cc(-c6ccccc6)ccc5c4c3)c3ccc(-c4ccccc4)c4oc5ccccc5c34)c(-c3ccccc3)c2)cc1]

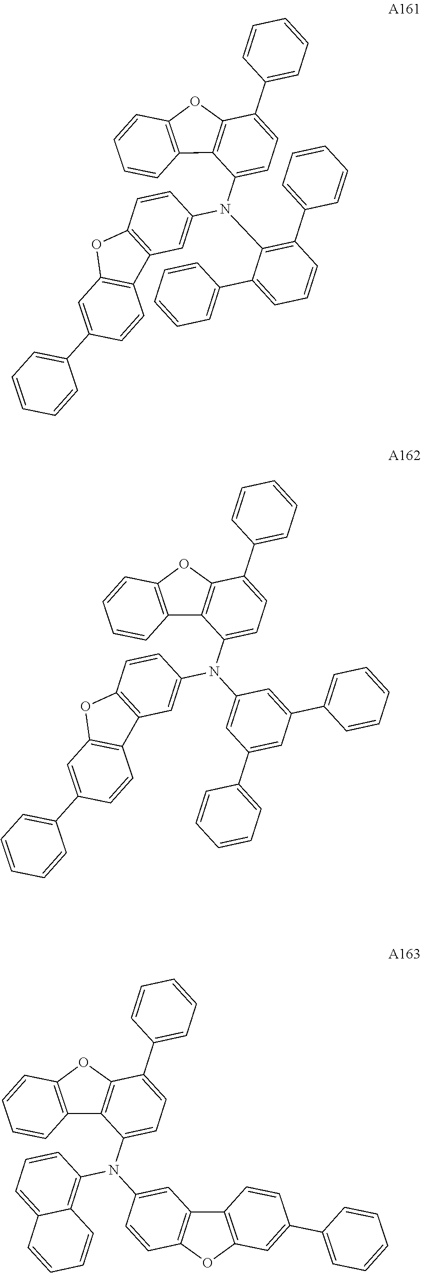 [SMILES: c1ccc(-c2ccc3c(c2)oc2ccc(N(c4c(-c5ccccc5)cccc4-c4ccccc4)c4ccc(-c5ccccc5)c5oc6ccccc6c45)cc23)cc1 ; c1ccc(-c2cc(-c3ccccc3)cc(N(c3ccc4oc5cc(-c6ccccc6)ccc5c4c3)c3ccc(-c4ccccc4)c4oc5ccccc5c34)c2)cc1 ; c1ccc(-c2ccc3c(c2)oc2ccc(N(c4cccc5ccccc45)c4ccc(-c5ccccc5)c5oc6ccccc6c45)cc23)cc1]

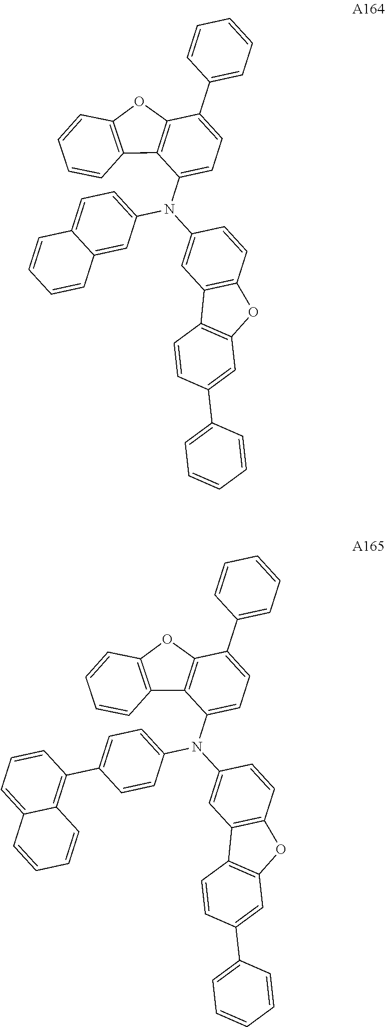 [SMILES: c1ccc(-c2ccc3c(c2)oc2ccc(N(c4ccc5ccccc5c4)c4ccc(-c5ccccc5)c5oc6ccccc6c45)cc23)cc1 ; c1ccc(-c2ccc3c(c2)oc2ccc(N(c4ccc(-c5cccc6ccccc56)cc4)c4ccc(-c5ccccc5)c5oc6ccccc6c45)cc23)cc1]

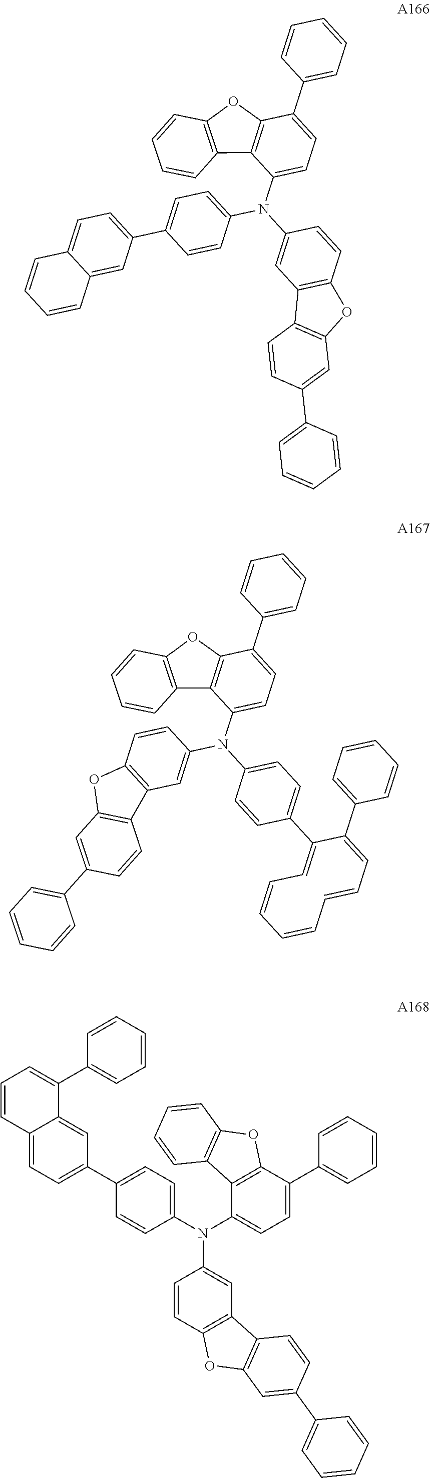 [SMILES: c1ccc(-c2ccc3c(c2)oc2ccc(N(c4ccc(-c5ccc6ccccc6c5)cc4)c4ccc(-c5ccccc5)c5oc6ccccc6c45)cc23)cc1 ; c1ccccc(-c2ccc(N(c3ccc4oc5cc(-c6ccccc6)ccc5c4c3)c3ccc(-c4ccccc4)c4oc5ccccc5c34)cc2)c(-c2ccccc2)ccc1 ; c1ccc(-c2ccc3c(c2)oc2ccc(N(c4ccc(-c5ccc6cccc(-c7ccccc7)c6c5)cc4)c4ccc(-c5ccccc5)c5oc6ccccc6c45)cc23)cc1]

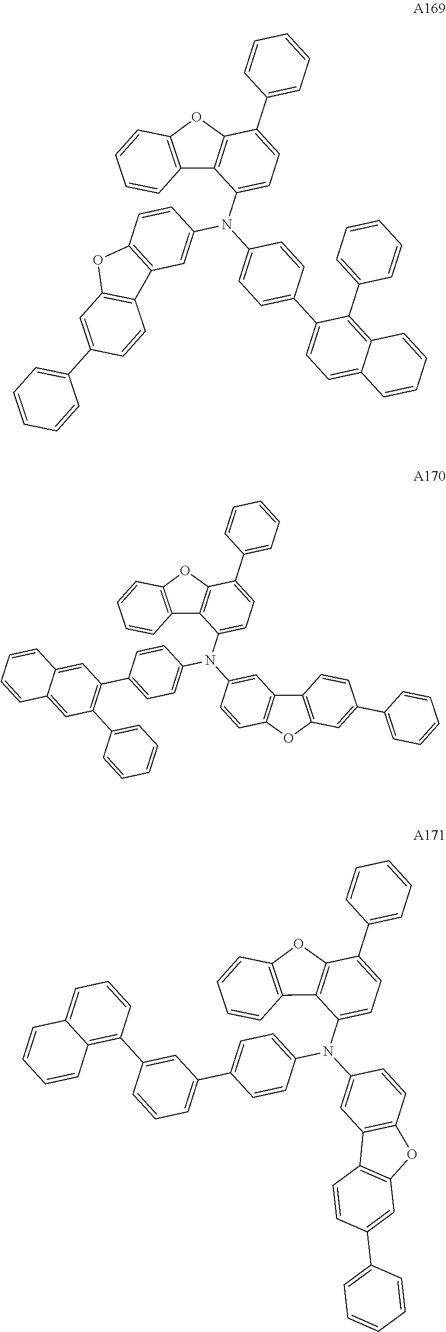 [SMILES: c1ccc(-c2ccc3c(c2)oc2ccc(N(c4ccc(-c5ccc6ccccc6c5-c5ccccc5)cc4)c4ccc(-c5ccccc5)c5oc6ccccc6c45)cc23)cc1 ; c1ccc(-c2ccc3c(c2)oc2ccc(N(c4ccc(-c5cc6ccccc6cc5-c5ccccc5)cc4)c4ccc(-c5ccccc5)c5oc6ccccc6c45)cc23)cc1 ; c1ccc(-c2ccc3c(c2)oc2ccc(N(c4ccc(-c5cccc(-c6cccc7ccccc67)c5)cc4)c4ccc(-c5ccccc5)c5oc6ccccc6c45)cc23)cc1]

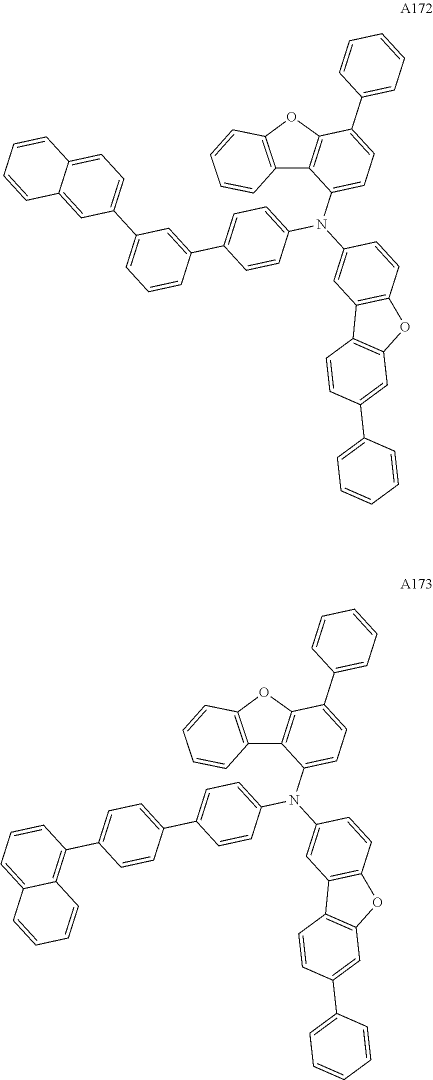 [SMILES: c1ccc(-c2ccc3c(c2)oc2ccc(N(c4ccc(-c5cccc(-c6ccc7ccccc7c6)c5)cc4)c4ccc(-c5ccccc5)c5oc6ccccc6c45)cc23)cc1 ; c1ccc(-c2ccc3c(c2)oc2ccc(N(c4ccc(-c5ccc(-c6cccc7ccccc67)cc5)cc4)c4ccc(-c5ccccc5)c5oc6ccccc6c45)cc23)cc1]

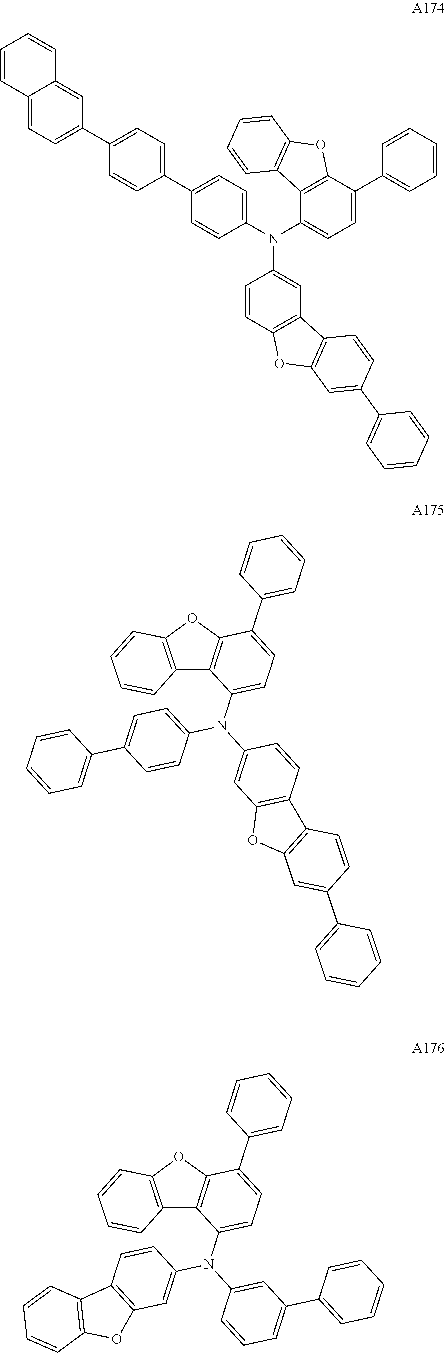 [SMILES: c1ccc(-c2ccc3c(c2)oc2ccc(N(c4ccc(-c5ccc(-c6ccc7ccccc7c6)cc5)cc4)c4ccc(-c5ccccc5)c5oc6ccccc6c45)cc23)cc1 ; c1ccc(-c2ccc(N(c3ccc4c(c3)oc3cc(-c5ccccc5)ccc34)c3ccc(-c4ccccc4)c4oc5ccccc5c34)cc2)cc1 ; c1ccc(-c2cccc(N(c3ccc4c(c3)oc3ccccc34)c3ccc(-c4ccccc4)c4oc5ccccc5c34)c2)cc1]

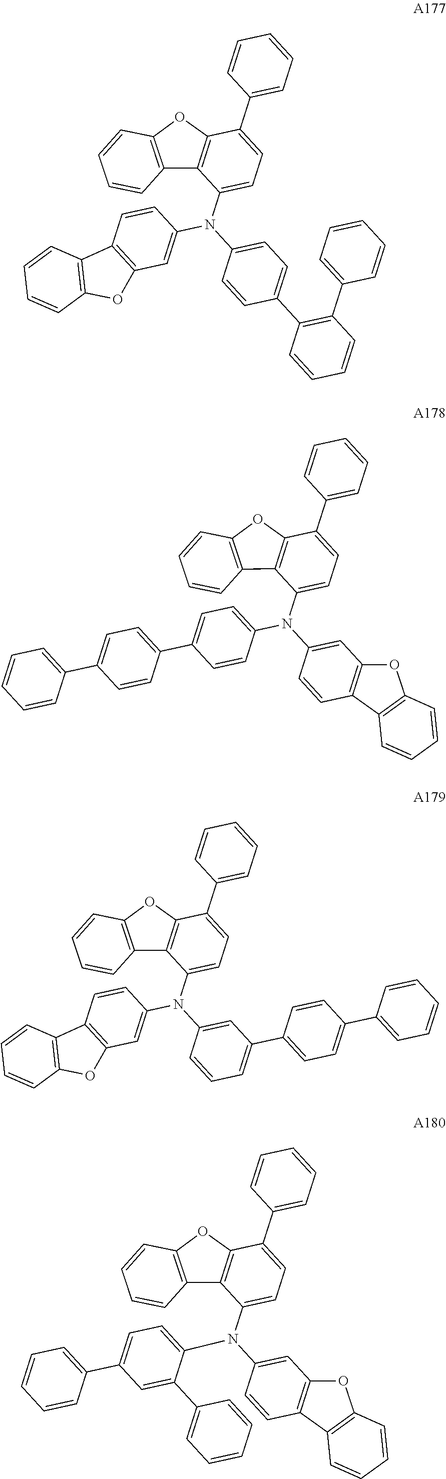 [SMILES: c1ccc(-c2ccccc2-c2ccc(N(c3ccc4c(c3)oc3ccccc34)c3ccc(-c4ccccc4)c4oc5ccccc5c34)cc2)cc1 ; c1ccc(-c2ccc(-c3ccc(N(c4ccc5c(c4)oc4ccccc45)c4ccc(-c5ccccc5)c5oc6ccccc6c45)cc3)cc2)cc1 ; c1ccc(-c2ccc(-c3cccc(N(c4ccc5c(c4)oc4ccccc45)c4ccc(-c5ccccc5)c5oc6ccccc6c45)c3)cc2)cc1 ; c1ccc(-c2ccc(N(c3ccc4c(c3)oc3ccccc34)c3ccc(-c4ccccc4)c4oc5ccccc5c34)c(-c3ccccc3)c2)cc1]

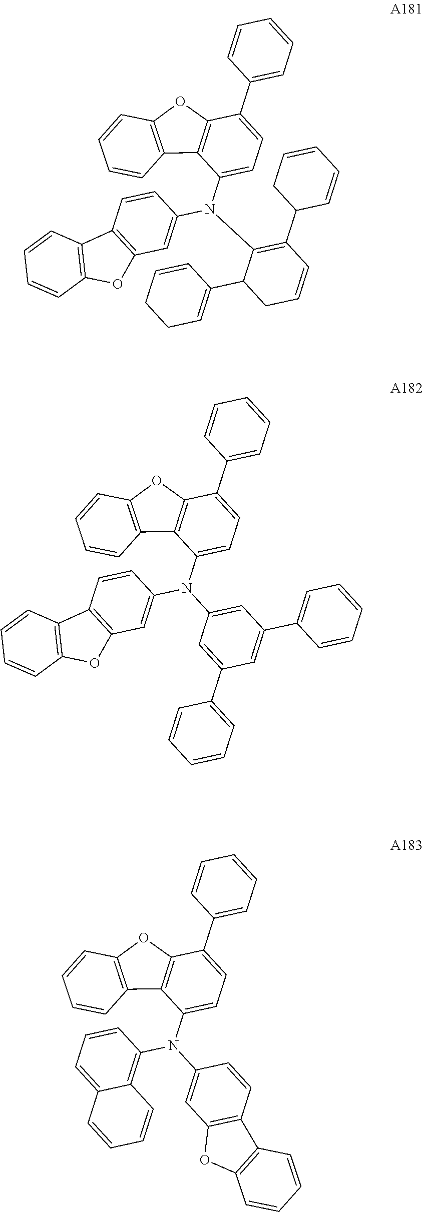 [SMILES: C1=CCC(C2=C(N(c3ccc4c(c3)oc3ccccc34)c3ccc(-c4ccccc4)c4oc5ccccc5c34)C(C3=CCCC=C3)CC=C2)C=C1 ; c1ccc(-c2cc(-c3ccccc3)cc(N(c3ccc4c(c3)oc3ccccc34)c3ccc(-c4ccccc4)c4oc5ccccc5c34)c2)cc1 ; c1ccc(-c2ccc(N(c3ccc4c(c3)oc3ccccc34)c3cccc4ccccc34)c3c2oc2ccccc23)cc1]

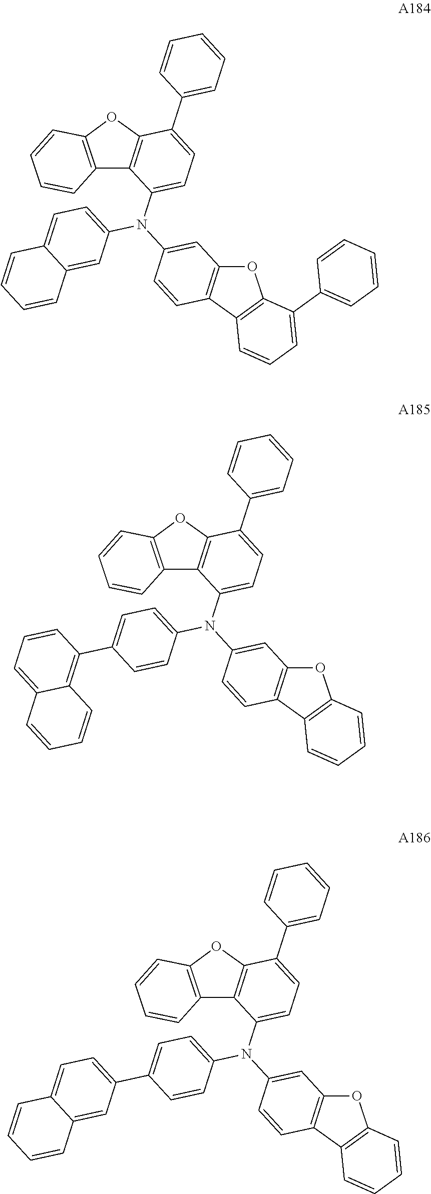 [SMILES: c1ccc(-c2cccc3c2oc2cc(N(c4ccc5ccccc5c4)c4ccc(-c5ccccc5)c5oc6ccccc6c45)ccc23)cc1 ; c1ccc(-c2ccc(N(c3ccc(-c4cccc5ccccc45)cc3)c3ccc4c(c3)oc3ccccc34)c3c2oc2ccccc23)cc1 ; c1ccc(-c2ccc(N(c3ccc(-c4ccc5ccccc5c4)cc3)c3ccc4c(c3)oc3ccccc34)c3c2oc2ccccc23)cc1]

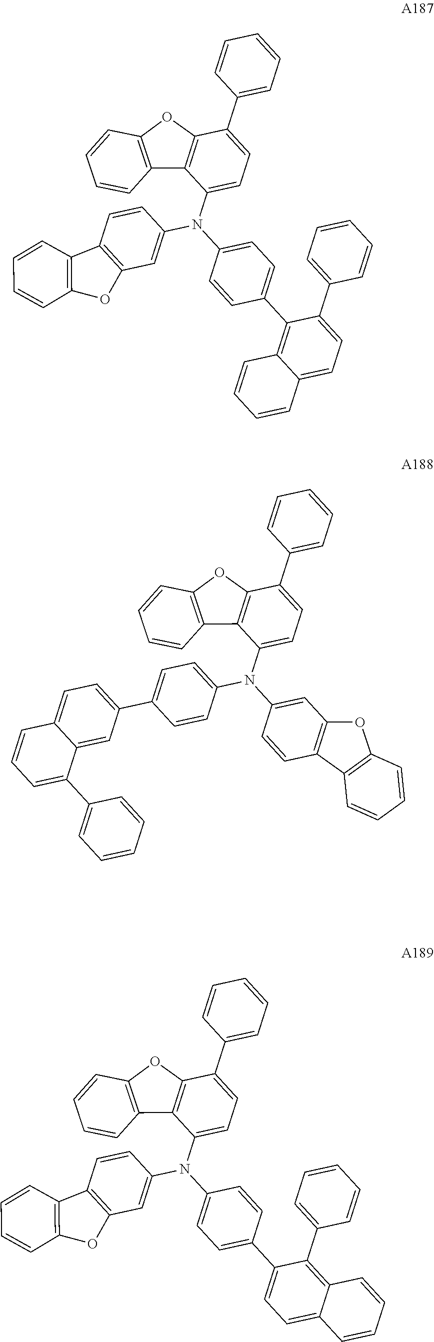 [SMILES: c1ccc(-c2ccc3ccccc3c2-c2ccc(N(c3ccc4c(c3)oc3ccccc34)c3ccc(-c4ccccc4)c4oc5ccccc5c34)cc2)cc1 ; c1ccc(-c2cccc3ccc(-c4ccc(N(c5ccc6c(c5)oc5ccccc56)c5ccc(-c6ccccc6)c6oc7ccccc7c56)cc4)cc23)cc1 ; c1ccc(-c2c(-c3ccc(N(c4ccc5c(c4)oc4ccccc45)c4ccc(-c5ccccc5)c5oc6ccccc6c45)cc3)ccc3ccccc23)cc1]

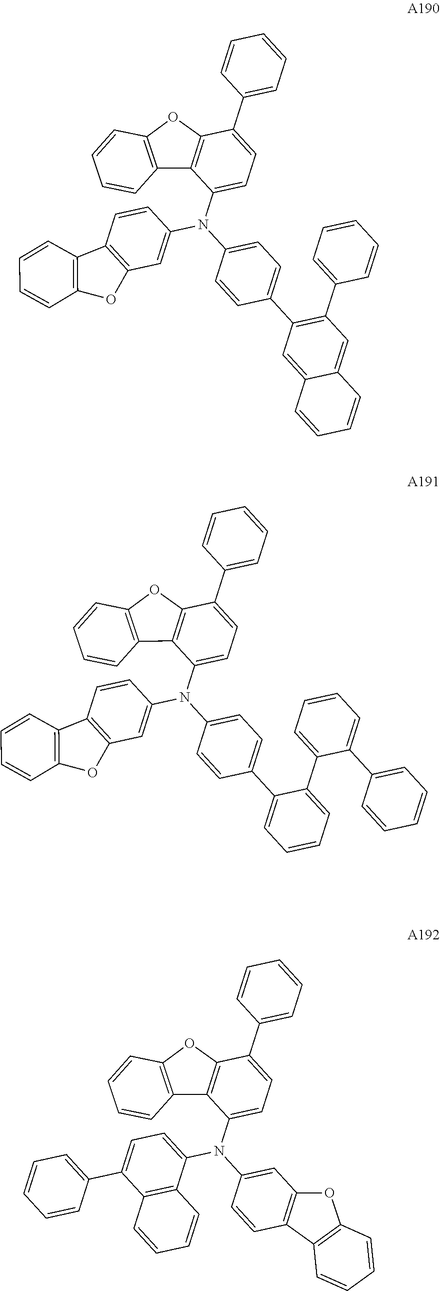 [SMILES: c1ccc(-c2cc3ccccc3cc2-c2ccc(N(c3ccc4c(c3)oc3ccccc34)c3ccc(-c4ccccc4)c4oc5ccccc5c34)cc2)cc1 ; c1ccc(-c2ccccc2-c2ccccc2-c2ccc(N(c3ccc4c(c3)oc3ccccc34)c3ccc(-c4ccccc4)c4oc5ccccc5c34)cc2)cc1 ; c1ccc(-c2ccc(N(c3ccc4c(c3)oc3ccccc34)c3ccc(-c4ccccc4)c4oc5ccccc5c34)c3ccccc23)cc1]

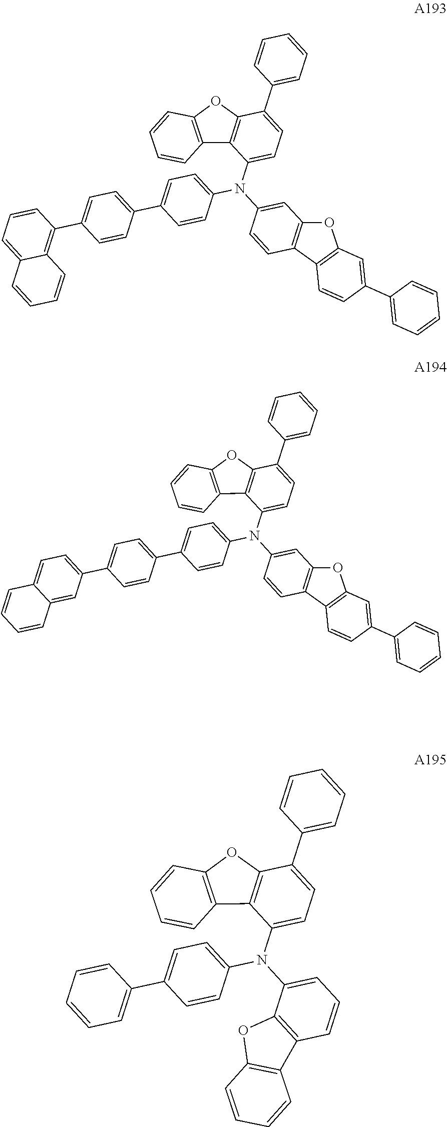 [SMILES: c1ccc(-c2ccc3c(c2)oc2cc(N(c4ccc(-c5ccc(-c6cccc7ccccc67)cc5)cc4)c4ccc(-c5ccccc5)c5oc6ccccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5oc6ccccc6c45)ccc23)cc1 ; c1ccc(-c2ccc(N(c3cccc4c3oc3ccccc34)c3ccc(-c4ccccc4)c4oc5ccccc5c34)cc2)cc1]

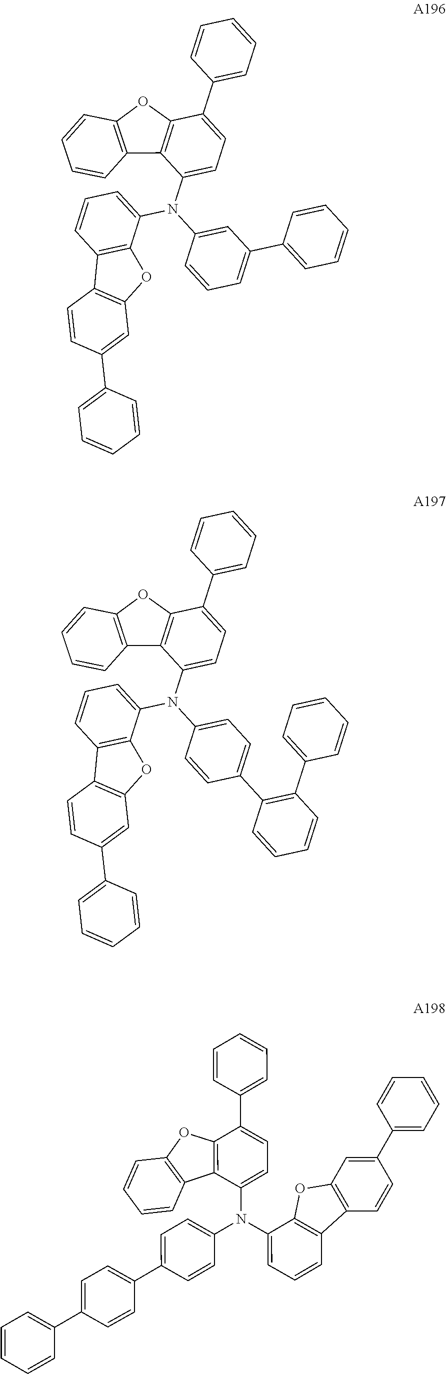 [SMILES: c1ccc(-c2cccc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4ccc(-c5ccccc5-c5ccccc5)cc4)c4ccc(-c5ccccc5)c5oc6ccccc6c45)cccc23)cc1 ; c1ccc(-c2ccc(-c3ccc(N(c4cccc5c4oc4cc(-c6ccccc6)ccc45)c4ccc(-c5ccccc5)c5oc6ccccc6c45)cc3)cc2)cc1]

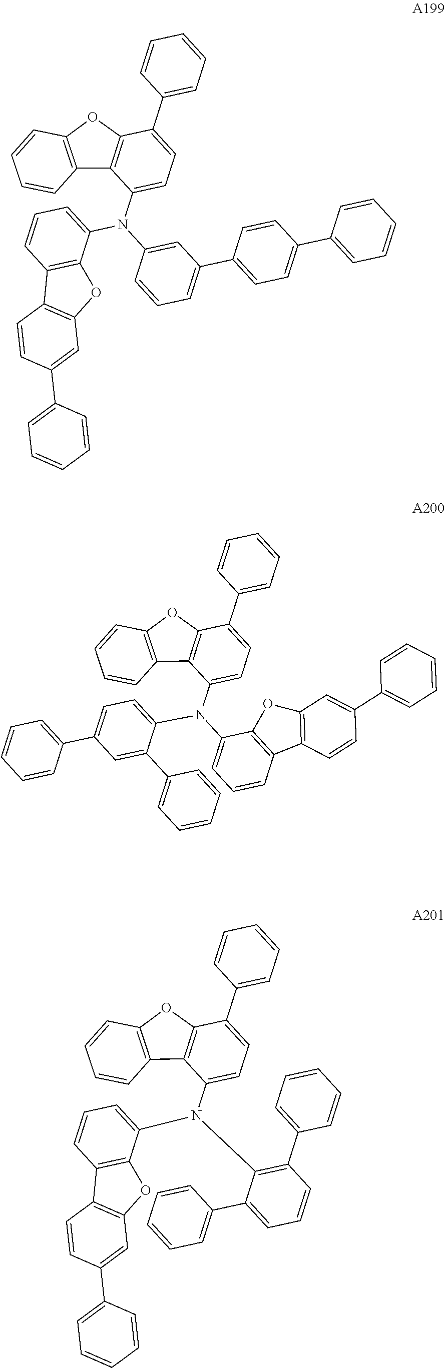 [SMILES: c1ccc(-c2ccc(-c3cccc(N(c4cccc5c4oc4cc(-c6ccccc6)ccc45)c4ccc(-c5ccccc5)c5oc6ccccc6c45)c3)cc2)cc1 ; c1ccc(-c2ccc(N(c3cccc4c3oc3cc(-c5ccccc5)ccc34)c3ccc(-c4ccccc4)c4oc5ccccc5c34)c(-c3ccccc3)c2)cc1 ; c1ccc(-c2ccc3c(c2)oc2c(N(c4c(-c5ccccc5)cccc4-c4ccccc4)c4ccc(-c5ccccc5)c5oc6ccccc6c45)cccc23)cc1]

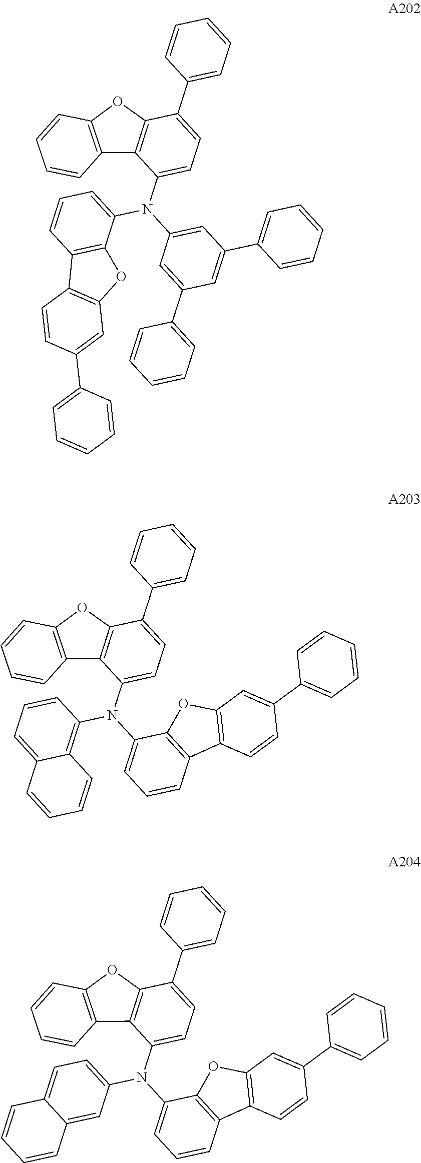 [SMILES: c1ccc(-c2cc(-c3ccccc3)cc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4cccc5ccccc45)c4ccc(-c5ccccc5)c5oc6ccccc6c45)cccc23)cc1 ; c1ccc(-c2ccc3c(c2)oc2c(N(c4ccc5ccccc5c4)c4ccc(-c5ccccc5)c5oc6ccccc6c45)cccc23)cc1]

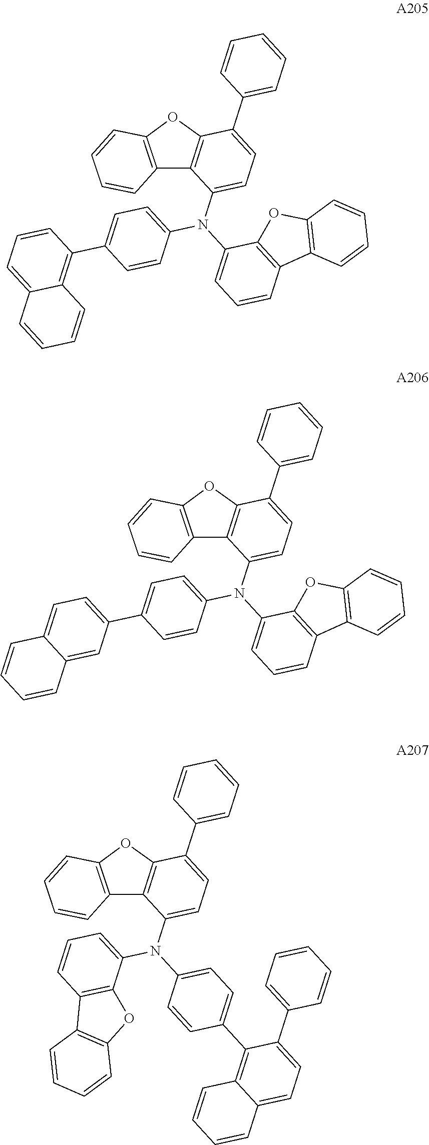 [SMILES: c1ccc(-c2ccc(N(c3ccc(-c4cccc5ccccc45)cc3)c3cccc4c3oc3ccccc34)c3c2oc2ccccc23)cc1 ; c1ccc(-c2ccc(N(c3ccc(-c4ccc5ccccc5c4)cc3)c3cccc4c3oc3ccccc34)c3c2oc2ccccc23)cc1 ; c1ccc(-c2ccc3ccccc3c2-c2ccc(N(c3cccc4c3oc3ccccc34)c3ccc(-c4ccccc4)c4oc5ccccc5c34)cc2)cc1]

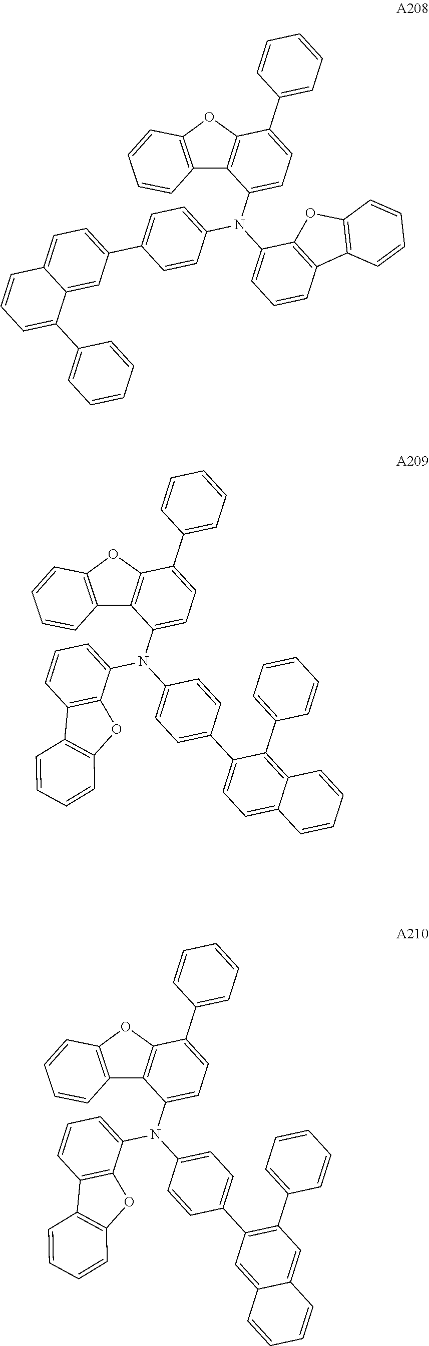 [SMILES: c1ccc(-c2cccc3ccc(-c4ccc(N(c5cccc6c5oc5ccccc56)c5ccc(-c6ccccc6)c6oc7ccccc7c56)cc4)cc23)cc1 ; c1ccc(-c2c(-c3ccc(N(c4cccc5c4oc4ccccc45)c4ccc(-c5ccccc5)c5oc6ccccc6c45)cc3)ccc3ccccc23)cc1 ; c1ccc(-c2cc3ccccc3cc2-c2ccc(N(c3cccc4c3oc3ccccc34)c3ccc(-c4ccccc4)c4oc5ccccc5c34)cc2)cc1]

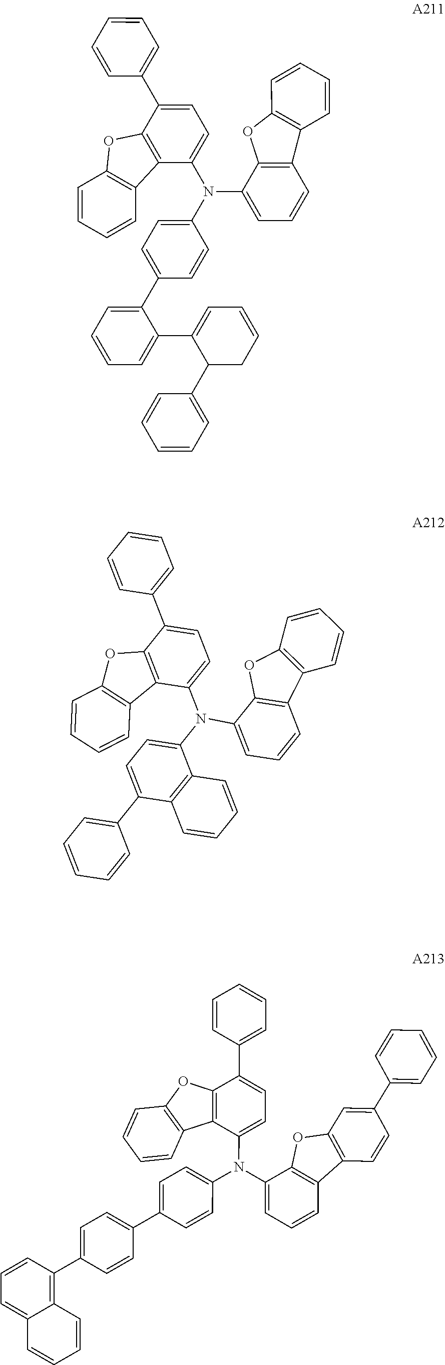 [SMILES: C1=CCC(c2ccccc2)C(c2ccccc2-c2ccc(N(c3cccc4c3oc3ccccc34)c3ccc(-c4ccccc4)c4oc5ccccc5c34)cc2)=C1 ; c1ccc(-c2ccc(N(c3cccc4c3oc3ccccc34)c3ccc(-c4ccccc4)c4oc5ccccc5c34)c3ccccc23)cc1 ; c1ccc(-c2ccc3c(c2)oc2c(N(c4ccc(-c5ccc(-c6cccc7ccccc67)cc5)cc4)c4ccc(-c5ccccc5)c5oc6ccccc6c45)cccc23)cc1]

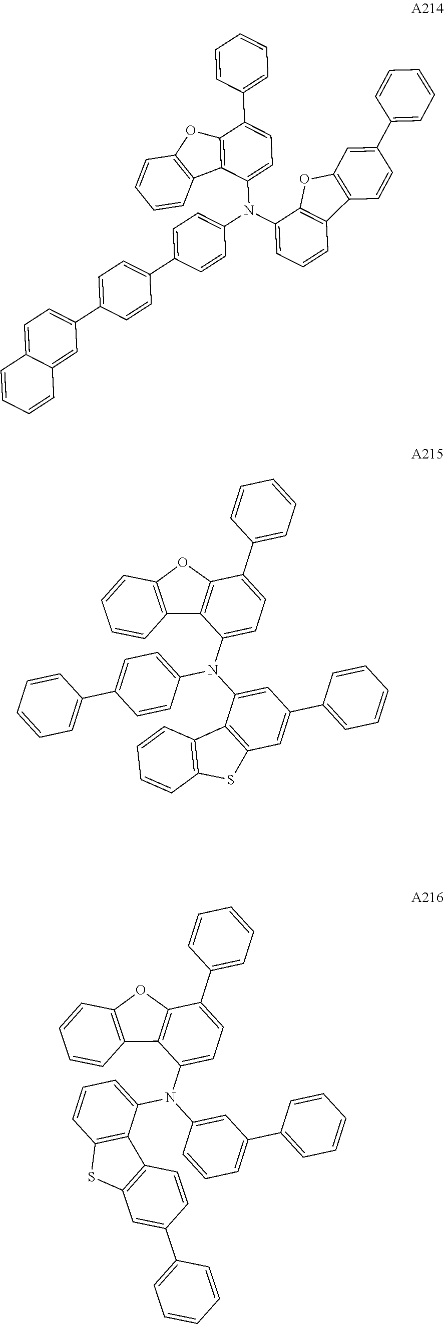 [SMILES: c1ccc(-c2ccc3c(c2)oc2c(N(c4ccc(-c5ccc(-c6ccc7ccccc7c6)cc5)cc4)c4ccc(-c5ccccc5)c5oc6ccccc6c45)cccc23)cc1 ; c1ccc(-c2ccc(N(c3cc(-c4ccccc4)cc4sc5ccccc5c34)c3ccc(-c4ccccc4)c4oc5ccccc5c34)cc2)cc1 ; c1ccc(-c2cccc(N(c3cccc4sc5cc(-c6ccccc6)ccc5c34)c3ccc(-c4ccccc4)c4oc5ccccc5c34)c2)cc1]

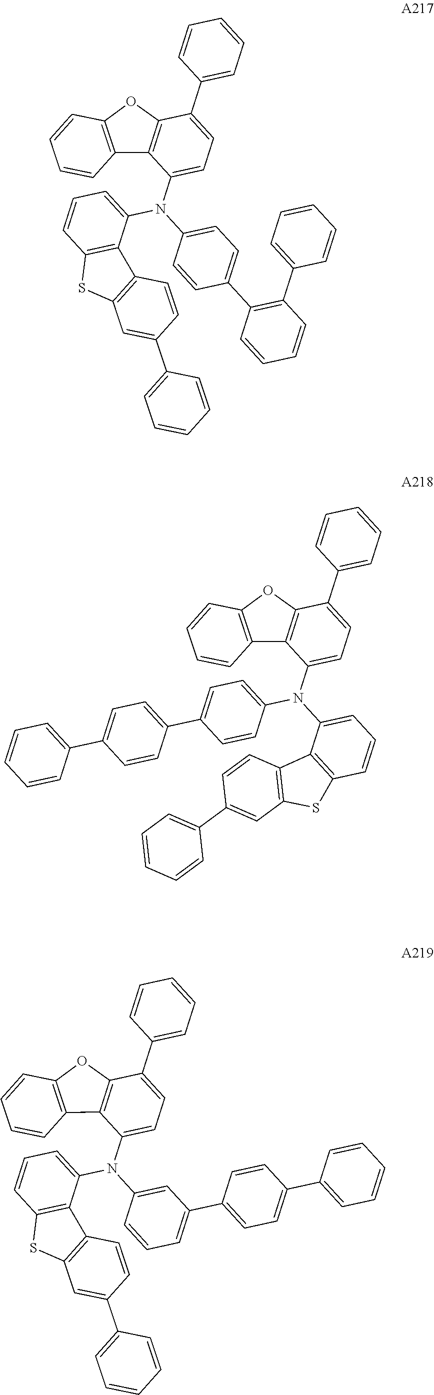 [SMILES: c1ccc(-c2ccc3c(c2)sc2cccc(N(c4ccc(-c5ccccc5-c5ccccc5)cc4)c4ccc(-c5ccccc5)c5oc6ccccc6c45)c23)cc1 ; c1ccc(-c2ccc(-c3ccc(N(c4cccc5sc6cc(-c7ccccc7)ccc6c45)c4ccc(-c5ccccc5)c5oc6ccccc6c45)cc3)cc2)cc1 ; c1ccc(-c2ccc(-c3cccc(N(c4cccc5sc6cc(-c7ccccc7)ccc6c45)c4ccc(-c5ccccc5)c5oc6ccccc6c45)c3)cc2)cc1]

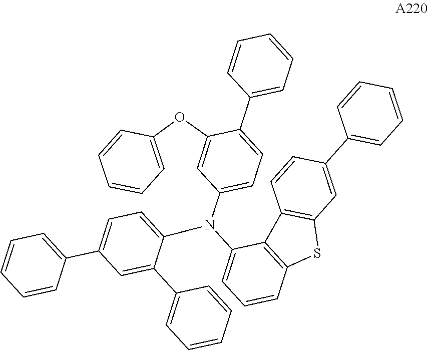 [SMILES: c1ccc(Oc2cc(N(c3ccc(-c4ccccc4)cc3-c3ccccc3)c3cccc4sc5cc(-c6ccccc6)ccc5c34)ccc2-c2ccccc2)cc1]

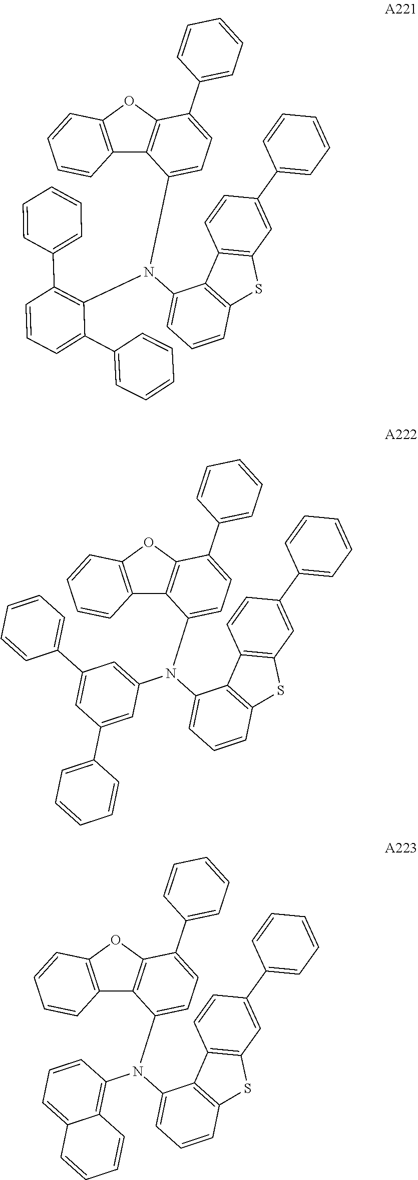 [SMILES: c1ccc(-c2ccc3c(c2)sc2cccc(N(c4c(-c5ccccc5)cccc4-c4ccccc4)c4ccc(-c5ccccc5)c5oc6ccccc6c45)c23)cc1 ; c1ccc(-c2cc(-c3ccccc3)cc(N(c3cccc4sc5cc(-c6ccccc6)ccc5c34)c3ccc(-c4ccccc4)c4oc5ccccc5c34)c2)cc1 ; c1ccc(-c2ccc3c(c2)sc2cccc(N(c4cccc5ccccc45)c4ccc(-c5ccccc5)c5oc6ccccc6c45)c23)cc1]

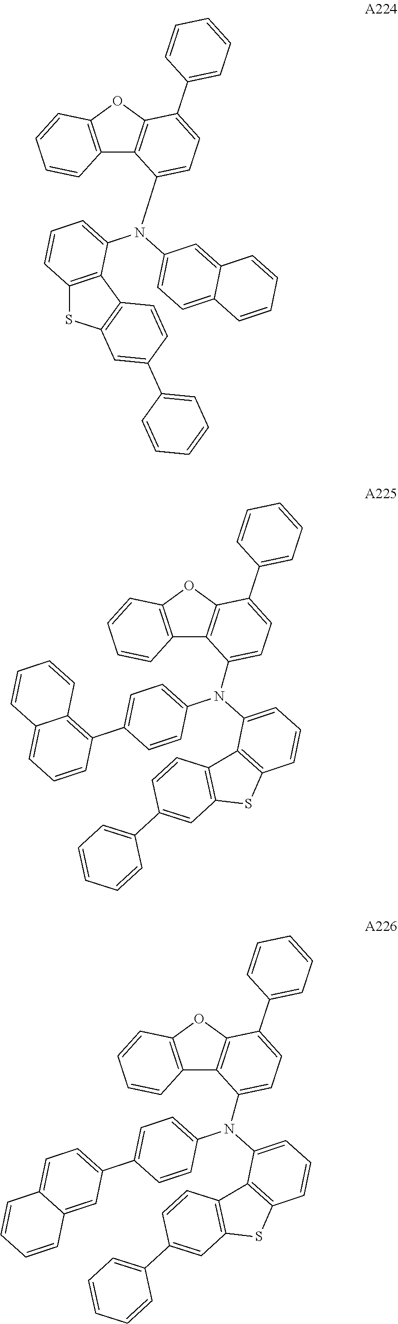 [SMILES: c1ccc(-c2ccc3c(c2)sc2cccc(N(c4ccc5ccccc5c4)c4ccc(-c5ccccc5)c5oc6ccccc6c45)c23)cc1 ; c1ccc(-c2ccc3c(c2)sc2cccc(N(c4ccc(-c5cccc6ccccc56)cc4)c4ccc(-c5ccccc5)c5oc6ccccc6c45)c23)cc1 ; c1ccc(-c2ccc3c(c2)sc2cccc(N(c4ccc(-c5ccc6ccccc6c5)cc4)c4ccc(-c5ccccc5)c5oc6ccccc6c45)c23)cc1]

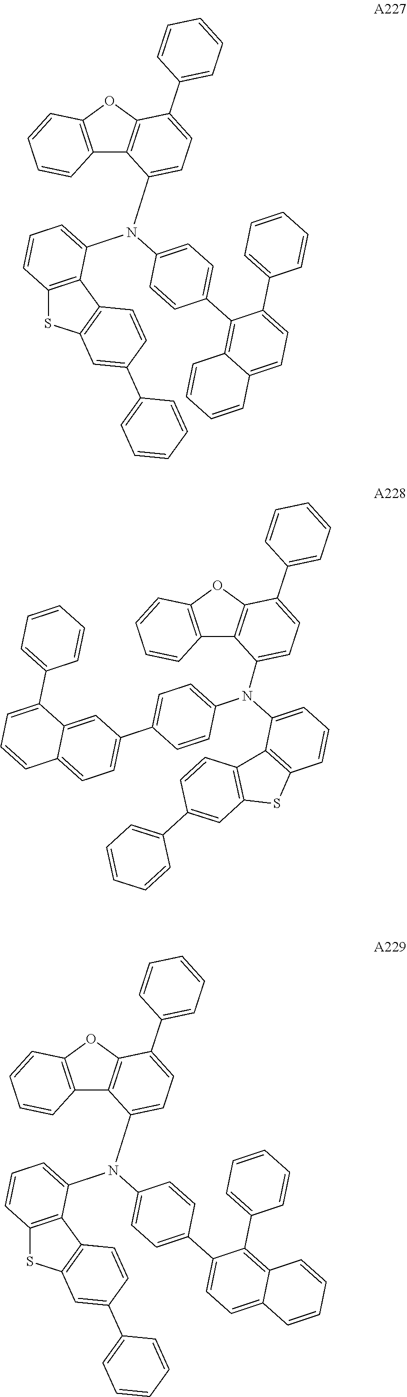 [SMILES: c1ccc(-c2ccc3c(c2)sc2cccc(N(c4ccc(-c5c(-c6ccccc6)ccc6ccccc56)cc4)c4ccc(-c5ccccc5)c5oc6ccccc6c45)c23)cc1 ; c1ccc(-c2ccc3c(c2)sc2cccc(N(c4ccc(-c5ccc6cccc(-c7ccccc7)c6c5)cc4)c4ccc(-c5ccccc5)c5oc6ccccc6c45)c23)cc1 ; c1ccc(-c2ccc3c(c2)sc2cccc(N(c4ccc(-c5ccc6ccccc6c5-c5ccccc5)cc4)c4ccc(-c5ccccc5)c5oc6ccccc6c45)c23)cc1]

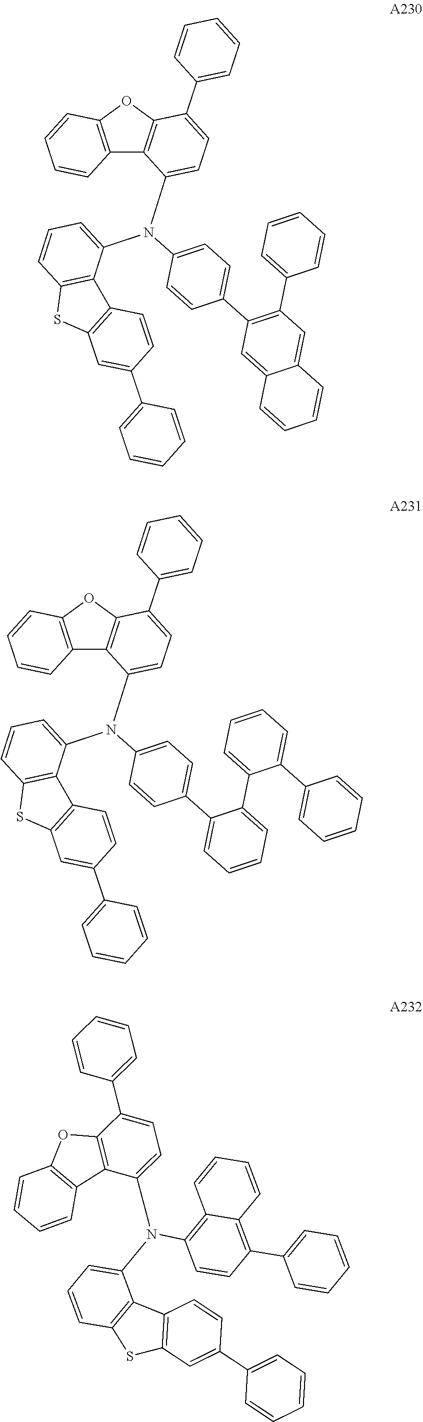 [SMILES: c1ccc(-c2ccc3c(c2)sc2cccc(N(c4ccc(-c5cc6ccccc6cc5-c5ccccc5)cc4)c4ccc(-c5ccccc5)c5oc6ccccc6c45)c23)cc1 ; c1ccc(-c2ccc3c(c2)sc2cccc(N(c4ccc(-c5ccccc5-c5ccccc5-c5ccccc5)cc4)c4ccc(-c5ccccc5)c5oc6ccccc6c45)c23)cc1 ; c1ccc(-c2ccc3c(c2)sc2cccc(N(c4ccc(-c5ccccc5)c5ccccc45)c4ccc(-c5ccccc5)c5oc6ccccc6c45)c23)cc1]

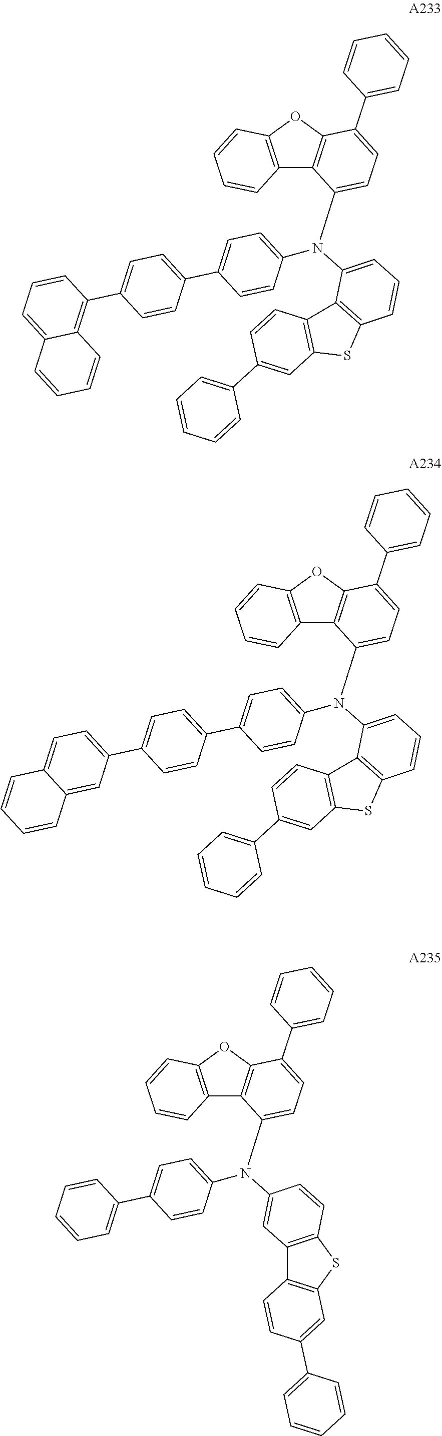 [SMILES: c1ccc(-c2ccc3c(c2)sc2cccc(N(c4ccc(-c5ccc(-c6cccc7ccccc67)cc5)cc4)c4ccc(-c5ccccc5)c5oc6ccccc6c45)c23)cc1 ; c1ccc(-c2ccc3c(c2)sc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4sc5cc(-c6ccccc6)ccc5c4c3)c3ccc(-c4ccccc4)c4oc5ccccc5c34)cc2)cc1]

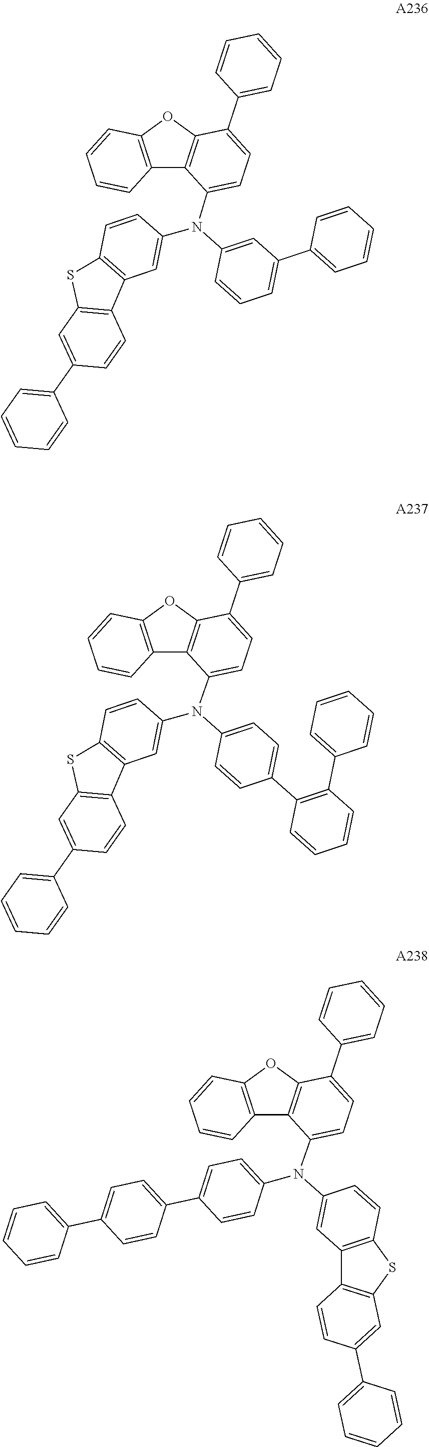 [SMILES: c1ccc(-c2cccc(N(c3ccc4sc5cc(-c6ccccc6)ccc5c4c3)c3ccc(-c4ccccc4)c4oc5ccccc5c34)c2)cc1 ; c1ccc(-c2ccc3c(c2)sc2ccc(N(c4ccc(-c5ccccc5-c5ccccc5)cc4)c4ccc(-c5ccccc5)c5oc6ccccc6c45)cc23)cc1 ; c1ccc(-c2ccc(-c3ccc(N(c4ccc5sc6cc(-c7ccccc7)ccc6c5c4)c4ccc(-c5ccccc5)c5oc6ccccc6c45)cc3)cc2)cc1]

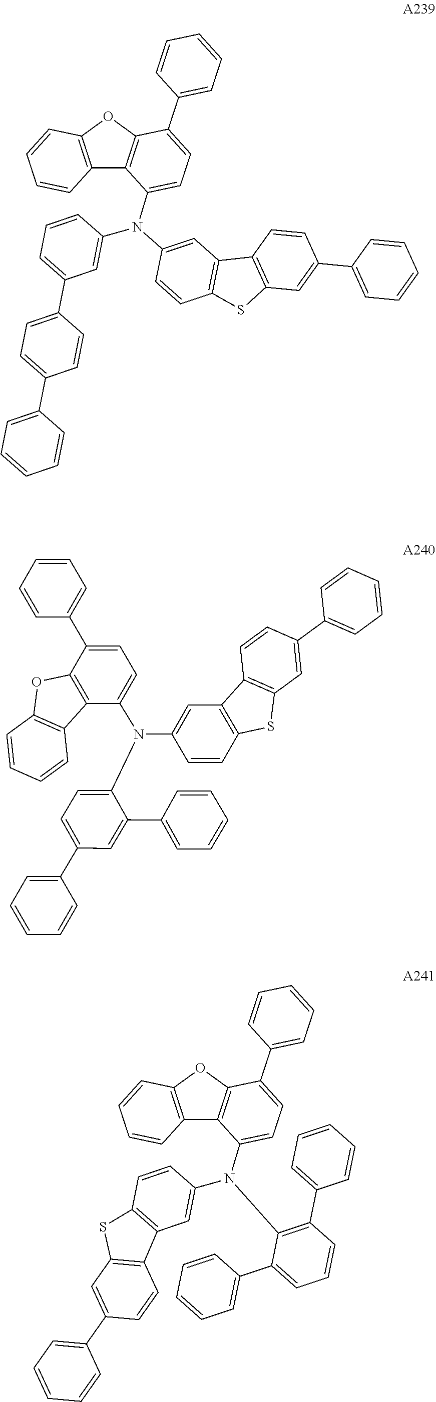 [SMILES: c1ccc(-c2ccc(-c3cccc(N(c4ccc5sc6cc(-c7ccccc7)ccc6c5c4)c4ccc(-c5ccccc5)c5oc6ccccc6c45)c3)cc2)cc1 ; c1ccc(-c2ccc(N(c3ccc4sc5cc(-c6ccccc6)ccc5c4c3)c3ccc(-c4ccccc4)c4oc5ccccc5c34)c(-c3ccccc3)c2)cc1 ; c1ccc(-c2ccc3c(c2)sc2ccc(N(c4c(-c5ccccc5)cccc4-c4ccccc4)c4ccc(-c5ccccc5)c5oc6ccccc6c45)cc23)cc1]

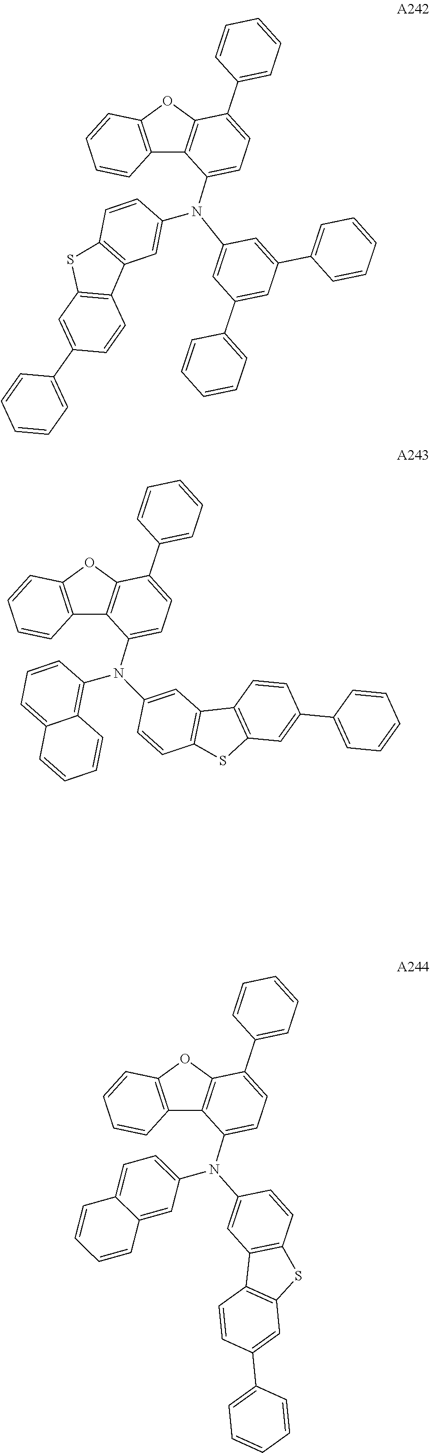 [SMILES: c1ccc(-c2cc(-c3ccccc3)cc(N(c3ccc4sc5cc(-c6ccccc6)ccc5c4c3)c3ccc(-c4ccccc4)c4oc5ccccc5c34)c2)cc1 ; c1ccc(-c2ccc3c(c2)sc2ccc(N(c4cccc5ccccc45)c4ccc(-c5ccccc5)c5oc6ccccc6c45)cc23)cc1 ; c1ccc(-c2ccc3c(c2)sc2ccc(N(c4ccc5ccccc5c4)c4ccc(-c5ccccc5)c5oc6ccccc6c45)cc23)cc1]

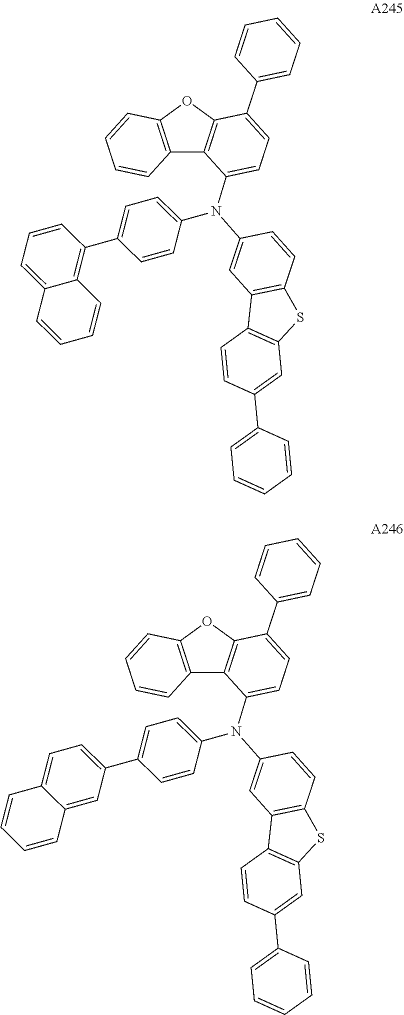 [SMILES: c1ccc(-c2ccc3c(c2)sc2ccc(N(c4ccc(-c5cccc6ccccc56)cc4)c4ccc(-c5ccccc5)c5oc6ccccc6c45)cc23)cc1 ; c1ccc(-c2ccc3c(c2)sc2ccc(N(c4ccc(-c5ccc6ccccc6c5)cc4)c4ccc(-c5ccccc5)c5oc6ccccc6c45)cc23)cc1]

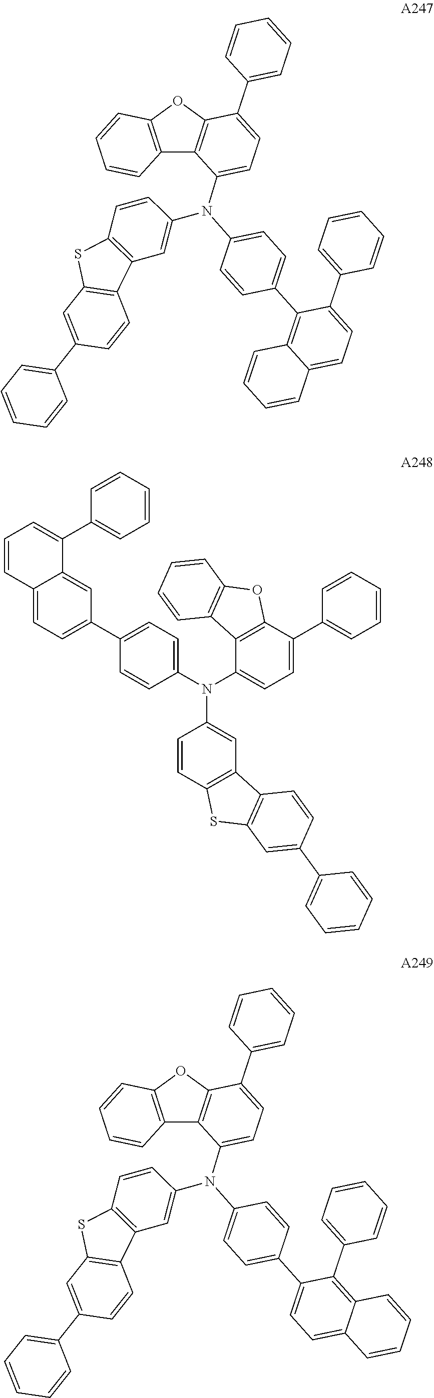 [SMILES: c1ccc(-c2ccc3c(c2)sc2ccc(N(c4ccc(-c5c(-c6ccccc6)ccc6ccccc56)cc4)c4ccc(-c5ccccc5)c5oc6ccccc6c45)cc23)cc1 ; c1ccc(-c2ccc3c(c2)sc2ccc(N(c4ccc(-c5ccc6cccc(-c7ccccc7)c6c5)cc4)c4ccc(-c5ccccc5)c5oc6ccccc6c45)cc23)cc1 ; c1ccc(-c2ccc3c(c2)sc2ccc(N(c4ccc(-c5ccc6ccccc6c5-c5ccccc5)cc4)c4ccc(-c5ccccc5)c5oc6ccccc6c45)cc23)cc1]

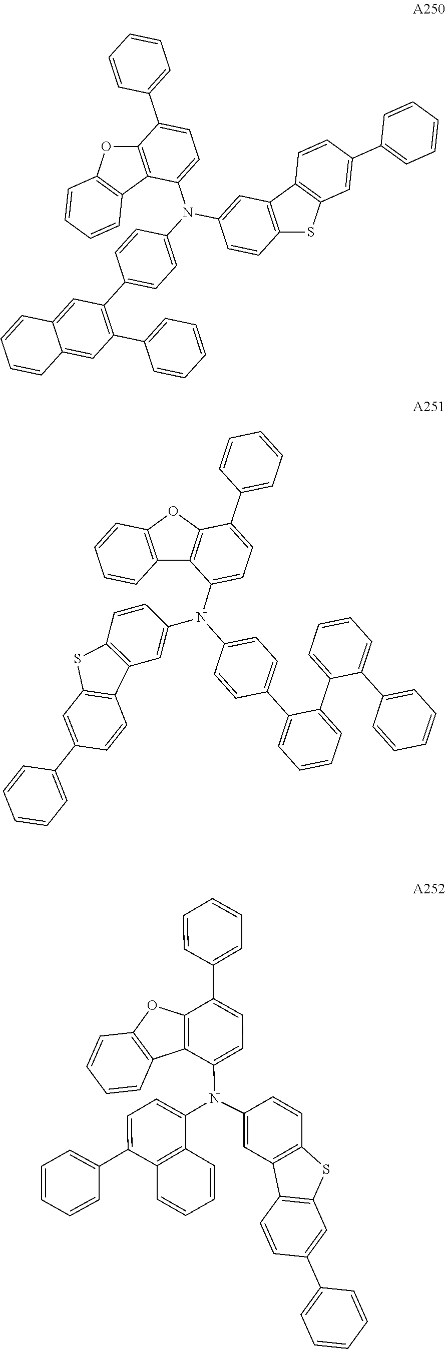 [SMILES: c1ccc(-c2ccc3c(c2)sc2ccc(N(c4ccc(-c5cc6ccccc6cc5-c5ccccc5)cc4)c4ccc(-c5ccccc5)c5oc6ccccc6c45)cc23)cc1 ; c1ccc(-c2ccc3c(c2)sc2ccc(N(c4ccc(-c5ccccc5-c5ccccc5-c5ccccc5)cc4)c4ccc(-c5ccccc5)c5oc6ccccc6c45)cc23)cc1 ; c1ccc(-c2ccc3c(c2)sc2ccc(N(c4ccc(-c5ccccc5)c5ccccc45)c4ccc(-c5ccccc5)c5oc6ccccc6c45)cc23)cc1]

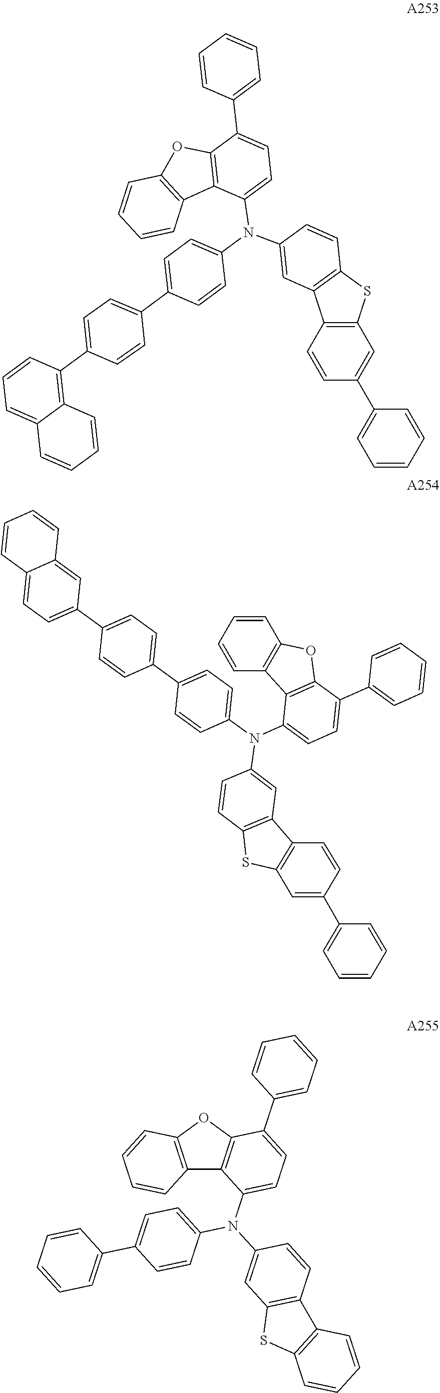 [SMILES: c1ccc(-c2ccc3c(c2)sc2ccc(N(c4ccc(-c5ccc(-c6cccc7ccccc67)cc5)cc4)c4ccc(-c5ccccc5)c5oc6ccccc6c45)cc23)cc1 ; c1ccc(-c2ccc3c(c2)sc2ccc(N(c4ccc(-c5ccc(-c6ccc7ccccc7c6)cc5)cc4)c4ccc(-c5ccccc5)c5oc6ccccc6c45)cc23)cc1 ; c1ccc(-c2ccc(N(c3ccc4c(c3)sc3ccccc34)c3ccc(-c4ccccc4)c4oc5ccccc5c34)cc2)cc1]

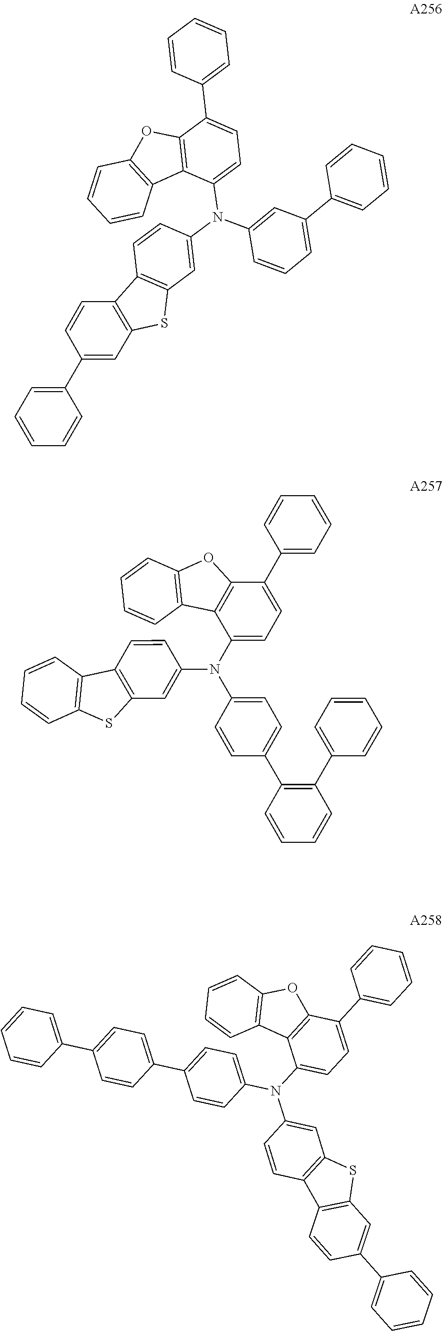 [SMILES: c1ccc(-c2cccc(N(c3ccc4c(c3)sc3cc(-c5ccccc5)ccc34)c3ccc(-c4ccccc4)c4oc5ccccc5c34)c2)cc1 ; c1ccc(-c2ccccc2-c2ccc(N(c3ccc4c(c3)sc3ccccc34)c3ccc(-c4ccccc4)c4oc5ccccc5c34)cc2)cc1 ; c1ccc(-c2ccc(-c3ccc(N(c4ccc5c(c4)sc4cc(-c6ccccc6)ccc45)c4ccc(-c5ccccc5)c5oc6ccccc6c45)cc3)cc2)cc1]

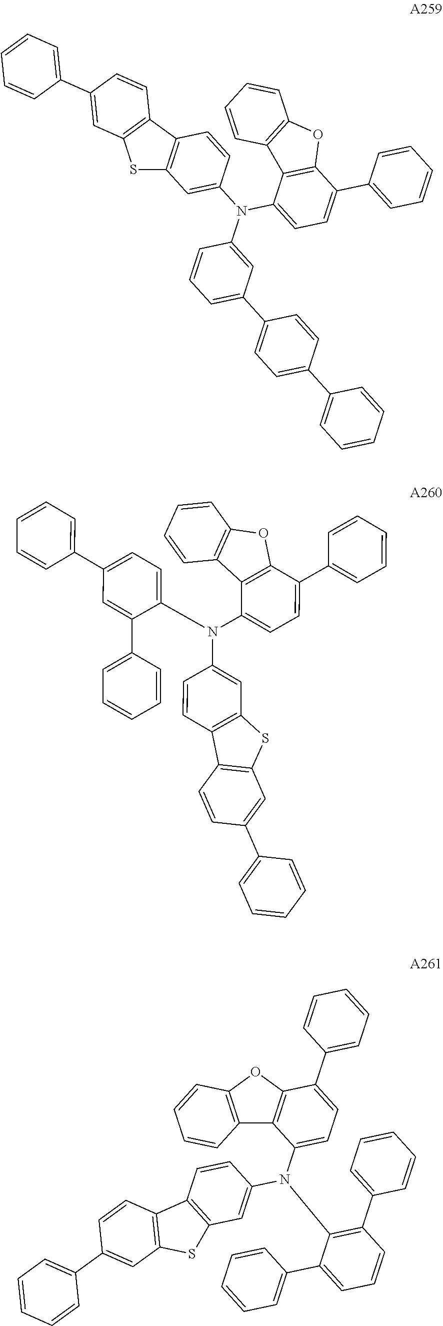 [SMILES: c1ccc(-c2ccc(-c3cccc(N(c4ccc5c(c4)sc4cc(-c6ccccc6)ccc45)c4ccc(-c5ccccc5)c5oc6ccccc6c45)c3)cc2)cc1 ; c1ccc(-c2ccc(N(c3ccc4c(c3)sc3cc(-c5ccccc5)ccc34)c3ccc(-c4ccccc4)c4oc5ccccc5c34)c(-c3ccccc3)c2)cc1 ; c1ccc(-c2ccc3c(c2)sc2cc(N(c4c(-c5ccccc5)cccc4-c4ccccc4)c4ccc(-c5ccccc5)c5oc6ccccc6c45)ccc23)cc1]

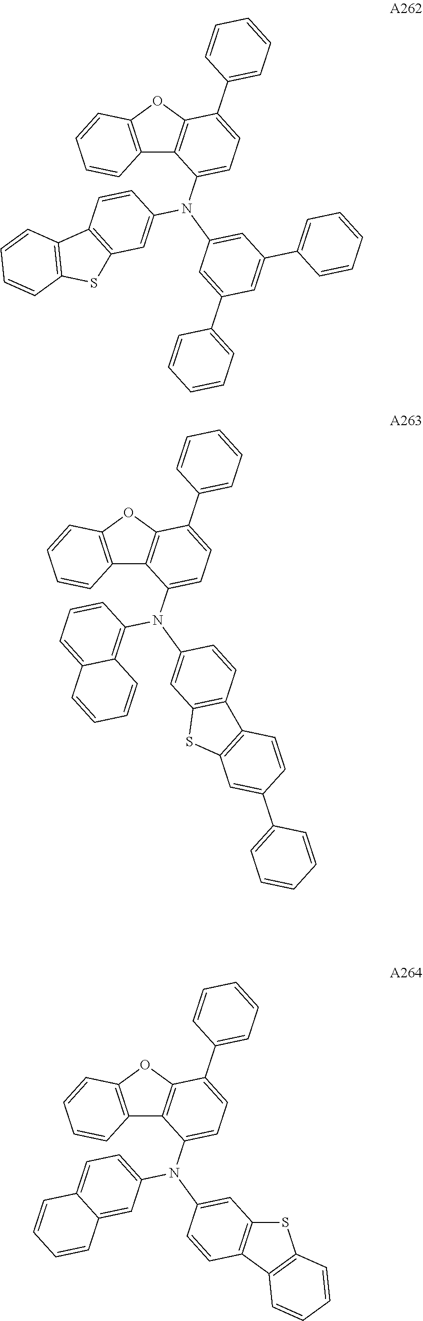

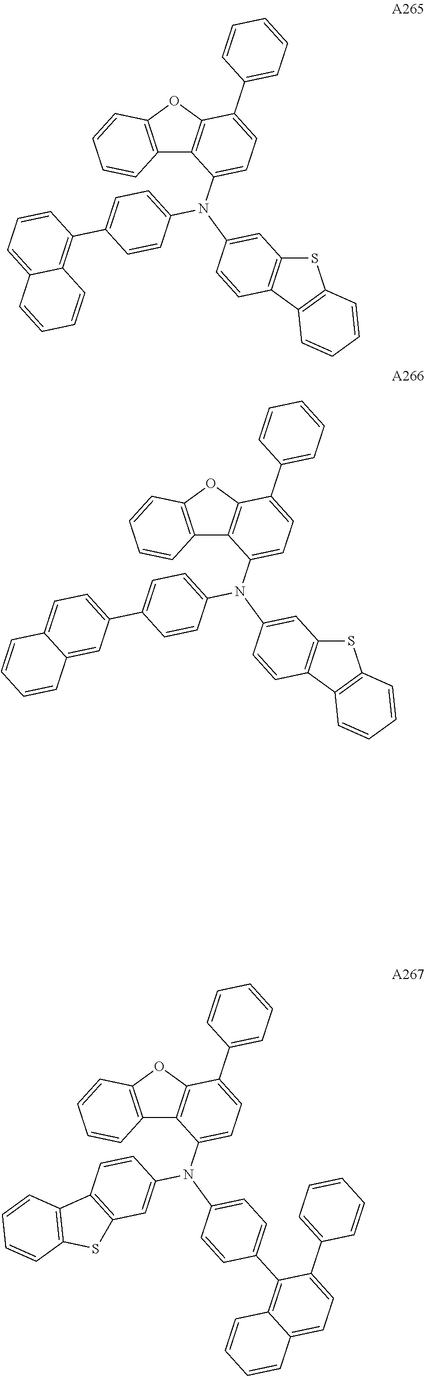 [SMILES: c1ccc(-c2ccc(N(c3ccc(-c4cccc5ccccc45)cc3)c3ccc4c(c3)sc3ccccc34)c3c2oc2ccccc23)cc1 ; c1ccc(-c2ccc(N(c3ccc(-c4ccc5ccccc5c4)cc3)c3ccc4c(c3)sc3ccccc34)c3c2oc2ccccc23)cc1 ; c1ccc(-c2ccc3ccccc3c2-c2ccc(N(c3ccc4c(c3)sc3ccccc34)c3ccc(-c4ccccc4)c4oc5ccccc5c34)cc2)cc1]

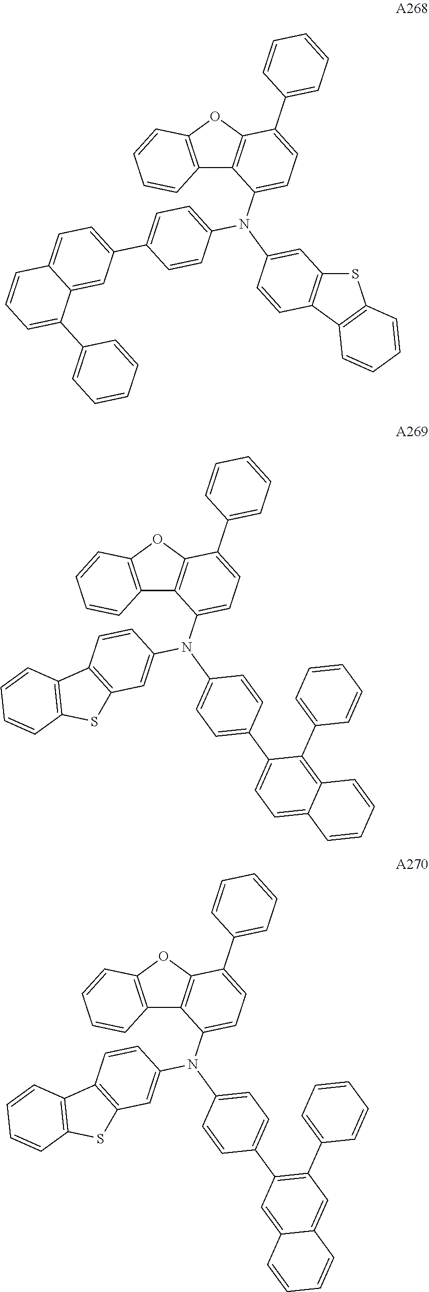 [SMILES: c1ccc(-c2cccc3ccc(-c4ccc(N(c5ccc6c(c5)sc5ccccc56)c5ccc(-c6ccccc6)c6oc7ccccc7c56)cc4)cc23)cc1 ; c1ccc(-c2c(-c3ccc(N(c4ccc5c(c4)sc4ccccc45)c4ccc(-c5ccccc5)c5oc6ccccc6c45)cc3)ccc3ccccc23)cc1 ; c1ccc(-c2cc3ccccc3cc2-c2ccc(N(c3ccc4c(c3)sc3ccccc34)c3ccc(-c4ccccc4)c4oc5ccccc5c34)cc2)cc1]

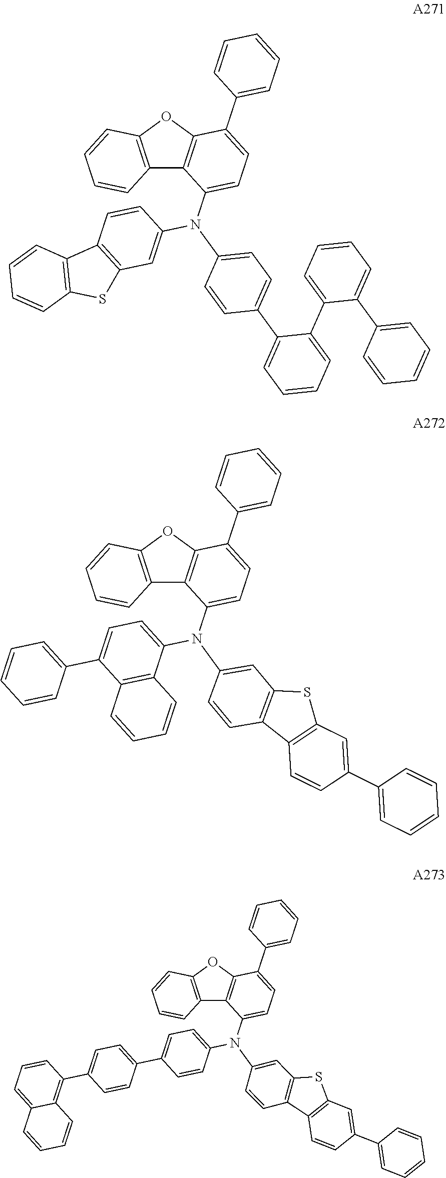 [SMILES: c1ccc(-c2ccccc2-c2ccccc2-c2ccc(N(c3ccc4c(c3)sc3ccccc34)c3ccc(-c4ccccc4)c4oc5ccccc5c34)cc2)cc1 ; c1ccc(-c2ccc3c(c2)sc2cc(N(c4ccc(-c5ccccc5)c5ccccc45)c4ccc(-c5ccccc5)c5oc6ccccc6c45)ccc23)cc1 ; c1ccc(-c2ccc3c(c2)sc2cc(N(c4ccc(-c5ccc(-c6cccc7ccccc67)cc5)cc4)c4ccc(-c5ccccc5)c5oc6ccccc6c45)ccc23)cc1]

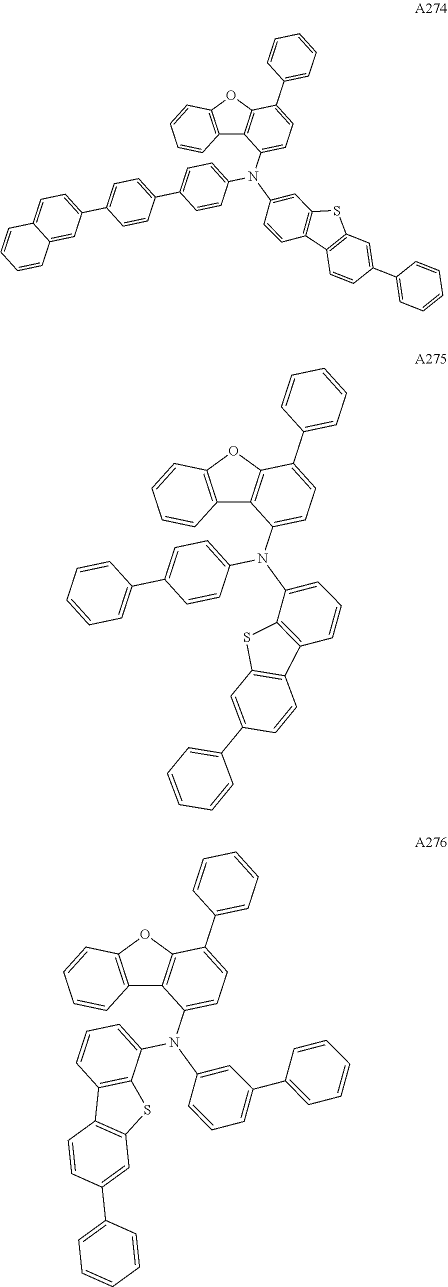 [SMILES: c1ccc(-c2ccc3c(c2)sc2cc(N(c4ccc(-c5ccc(-c6ccc7ccccc7c6)cc5)cc4)c4ccc(-c5ccccc5)c5oc6ccccc6c45)ccc23)cc1 ; c1ccc(-c2ccc(N(c3cccc4c3sc3cc(-c5ccccc5)ccc34)c3ccc(-c4ccccc4)c4oc5ccccc5c34)cc2)cc1 ; c1ccc(-c2cccc(N(c3cccc4c3sc3cc(-c5ccccc5)ccc34)c3ccc(-c4ccccc4)c4oc5ccccc5c34)c2)cc1]

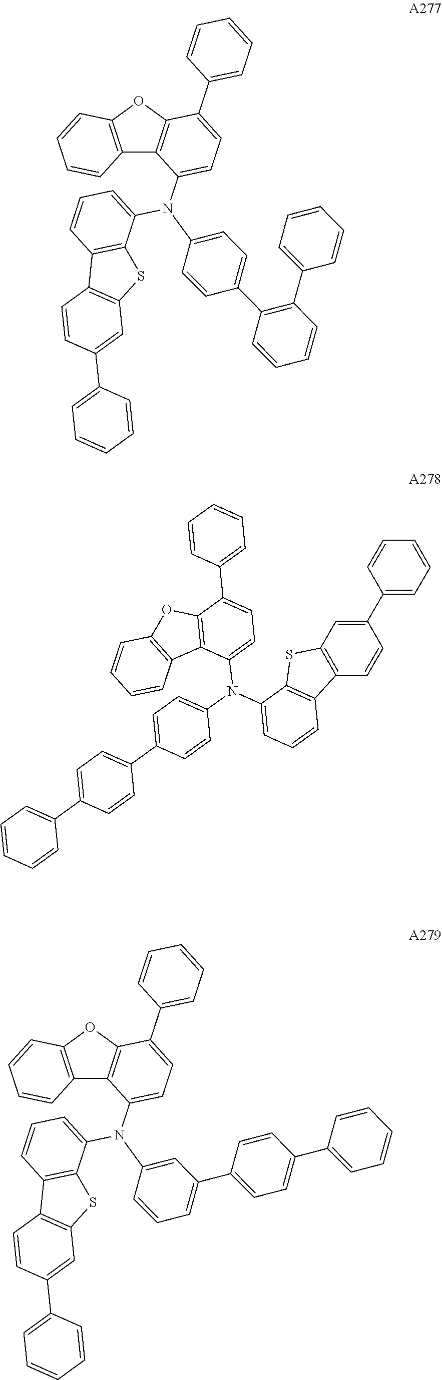 [SMILES: c1ccc(-c2ccc3c(c2)sc2c(N(c4ccc(-c5ccccc5-c5ccccc5)cc4)c4ccc(-c5ccccc5)c5oc6ccccc6c45)cccc23)cc1 ; c1ccc(-c2ccc(-c3ccc(N(c4cccc5c4sc4cc(-c6ccccc6)ccc45)c4ccc(-c5ccccc5)c5oc6ccccc6c45)cc3)cc2)cc1 ; c1ccc(-c2ccc(-c3cccc(N(c4cccc5c4sc4cc(-c6ccccc6)ccc45)c4ccc(-c5ccccc5)c5oc6ccccc6c45)c3)cc2)cc1]

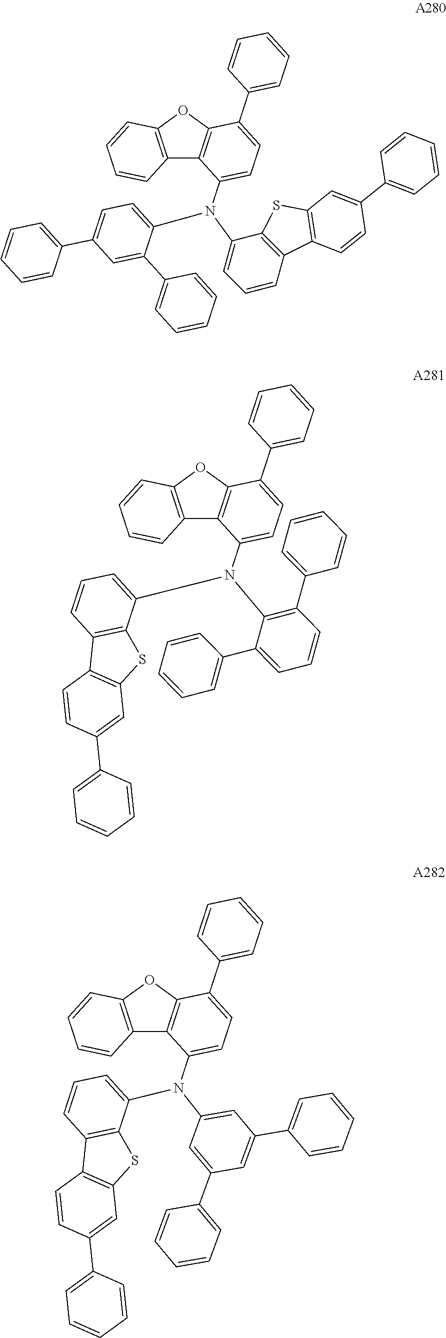 [SMILES: c1ccc(-c2ccc(N(c3cccc4c3sc3cc(-c5ccccc5)ccc34)c3ccc(-c4ccccc4)c4oc5ccccc5c34)c(-c3ccccc3)c2)cc1 ; c1ccc(-c2ccc3c(c2)sc2c(N(c4c(-c5ccccc5)cccc4-c4ccccc4)c4ccc(-c5ccccc5)c5oc6ccccc6c45)cccc23)cc1 ; c1ccc(-c2cc(-c3ccccc3)cc(N(c3cccc4c3sc3cc(-c5ccccc5)ccc34)c3ccc(-c4ccccc4)c4oc5ccccc5c34)c2)cc1]

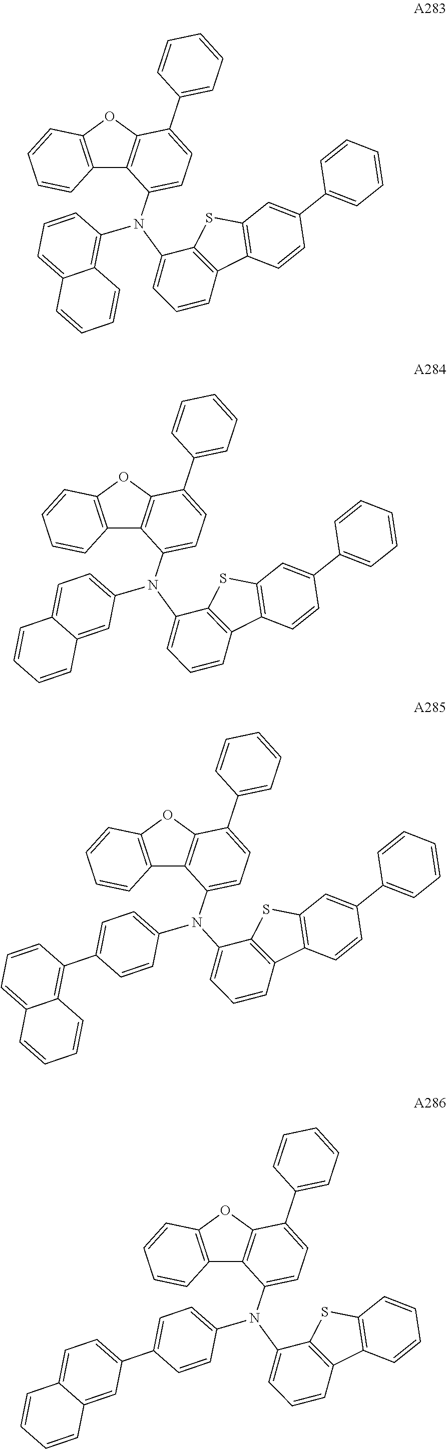 [SMILES: c1ccc(-c2ccc3c(c2)sc2c(N(c4cccc5ccccc45)c4ccc(-c5ccccc5)c5oc6ccccc6c45)cccc23)cc1 ; c1ccc(-c2ccc3c(c2)sc2c(N(c4ccc5ccccc5c4)c4ccc(-c5ccccc5)c5oc6ccccc6c45)cccc23)cc1 ; c1ccc(-c2ccc3c(c2)sc2c(N(c4ccc(-c5cccc6ccccc56)cc4)c4ccc(-c5ccccc5)c5oc6ccccc6c45)cccc23)cc1 ; c1ccc(-c2ccc(N(c3ccc(-c4ccc5ccccc5c4)cc3)c3cccc4c3sc3ccccc34)c3c2oc2ccccc23)cc1]

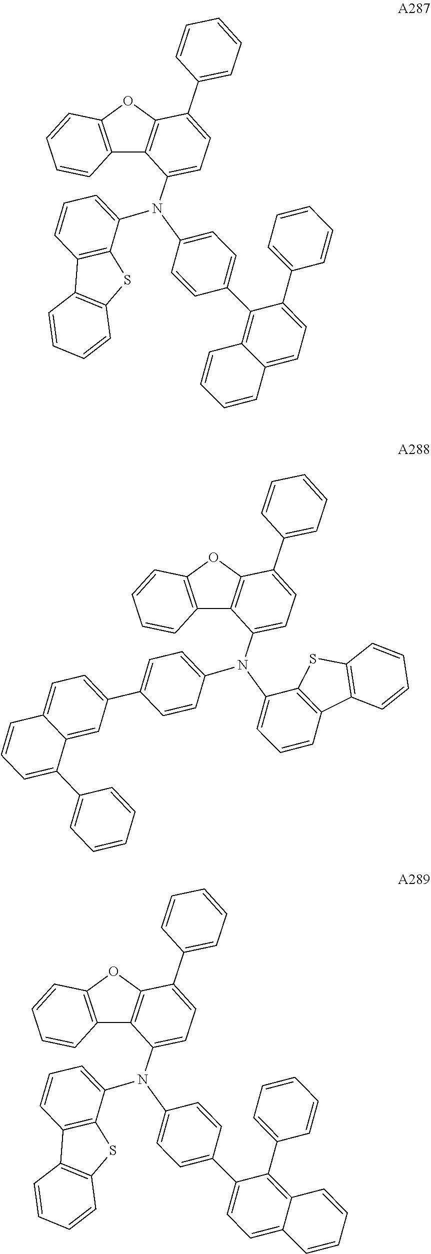 [SMILES: c1ccc(-c2ccc3ccccc3c2-c2ccc(N(c3cccc4c3sc3ccccc34)c3ccc(-c4ccccc4)c4oc5ccccc5c34)cc2)cc1 ; c1ccc(-c2cccc3ccc(-c4ccc(N(c5cccc6c5sc5ccccc56)c5ccc(-c6ccccc6)c6oc7ccccc7c56)cc4)cc23)cc1 ; c1ccc(-c2c(-c3ccc(N(c4cccc5c4sc4ccccc45)c4ccc(-c5ccccc5)c5oc6ccccc6c45)cc3)ccc3ccccc23)cc1]

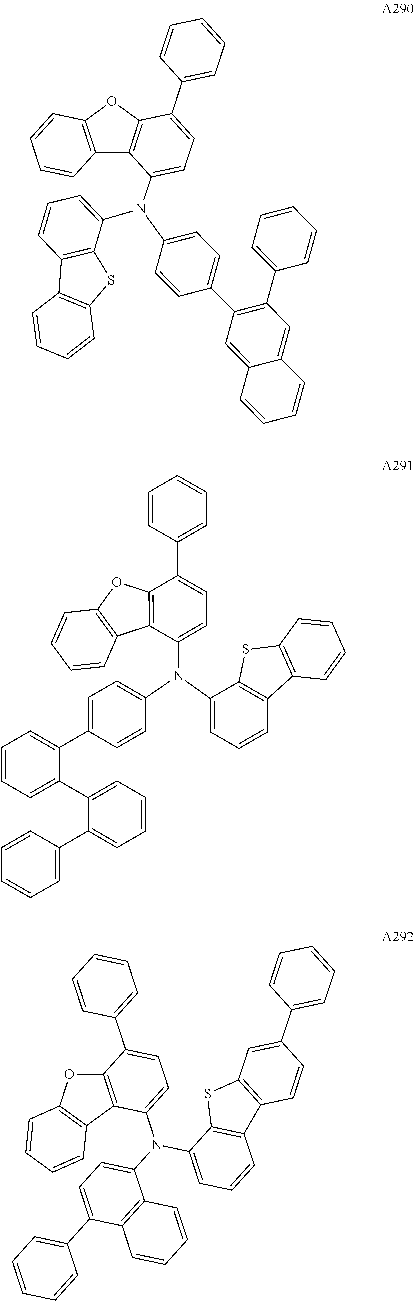 [SMILES: c1ccc(-c2cc3ccccc3cc2-c2ccc(N(c3cccc4c3sc3ccccc34)c3ccc(-c4ccccc4)c4oc5ccccc5c34)cc2)cc1 ; c1ccc(-c2ccccc2-c2ccccc2-c2ccc(N(c3cccc4c3sc3ccccc34)c3ccc(-c4ccccc4)c4oc5ccccc5c34)cc2)cc1 ; c1ccc(-c2ccc3c(c2)sc2c(N(c4ccc(-c5ccccc5)c5ccccc45)c4ccc(-c5ccccc5)c5oc6ccccc6c45)cccc23)cc1]

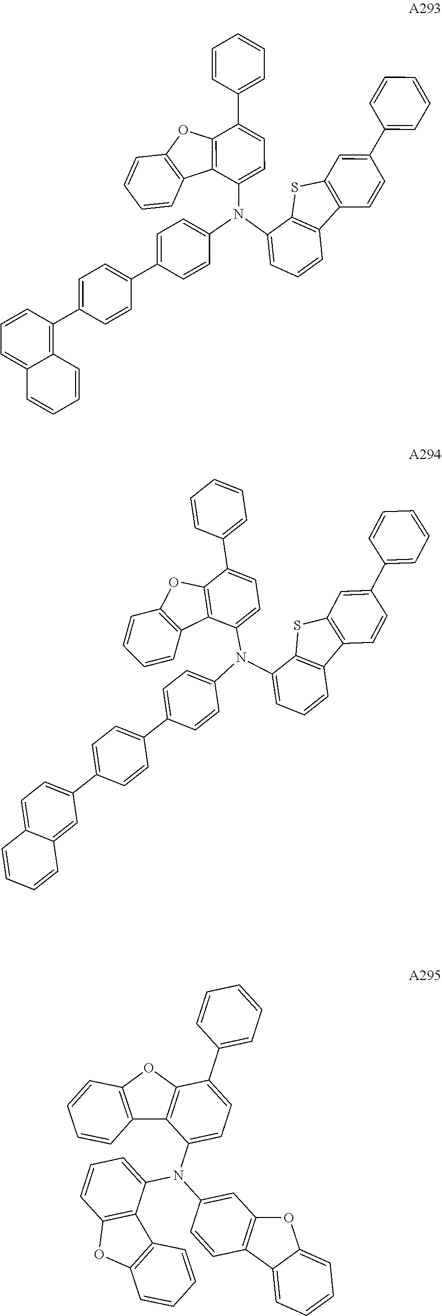 [SMILES: c1ccc(-c2ccc3c(c2)sc2c(N(c4ccc(-c5ccc(-c6cccc7ccccc67)cc5)cc4)c4ccc(-c5ccccc5)c5oc6ccccc6c45)cccc23)cc1 ; c1ccc(-c2ccc3c(c2)sc2c(N(c4ccc(-c5ccc(-c6ccc7ccccc7c6)cc5)cc4)c4ccc(-c5ccccc5)c5oc6ccccc6c45)cccc23)cc1 ; c1ccc(-c2ccc(N(c3ccc4c(c3)oc3ccccc34)c3cccc4oc5ccccc5c34)c3c2oc2ccccc23)cc1]

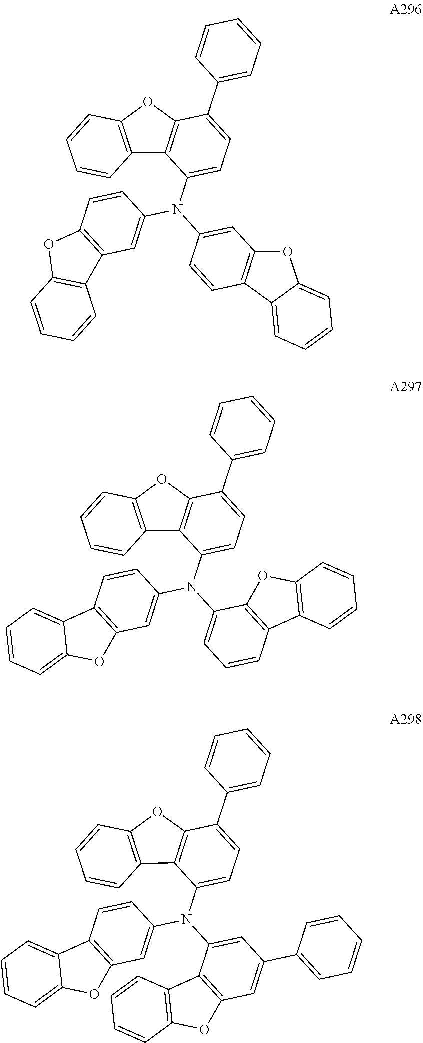 [SMILES: c1ccc(-c2ccc(N(c3ccc4c(c3)oc3ccccc34)c3ccc4oc5ccccc5c4c3)c3c2oc2ccccc23)cc1 ; c1ccc(-c2ccc(N(c3ccc4c(c3)oc3ccccc34)c3cccc4c3oc3ccccc34)c3c2oc2ccccc23)cc1 ; c1ccc(-c2cc(N(c3ccc4c(c3)oc3ccccc34)c3ccc(-c4ccccc4)c4oc5ccccc5c34)c3c(c2)oc2ccccc23)cc1]

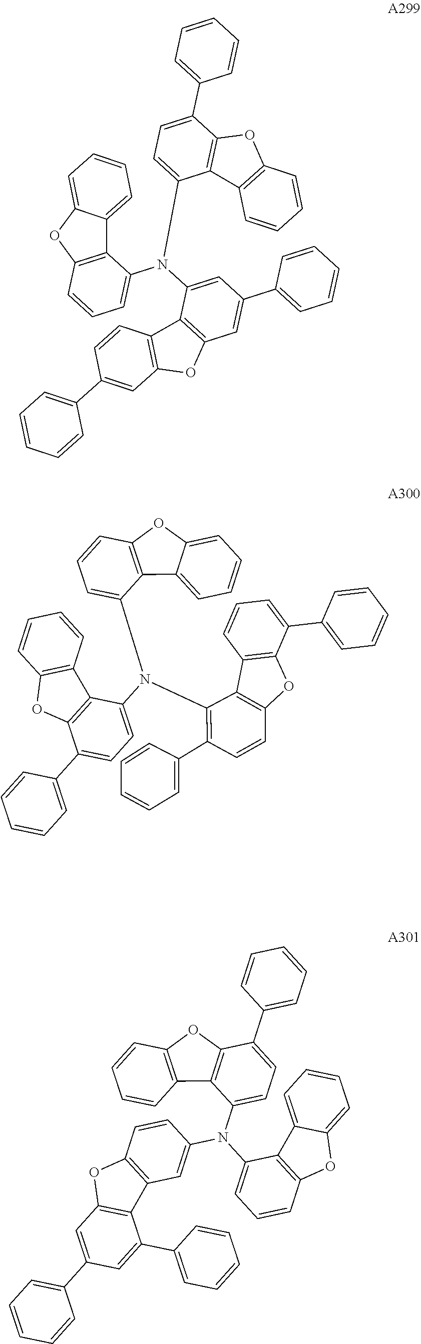 [SMILES: c1ccc(-c2ccc3c(c2)oc2cc(-c4ccccc4)cc(N(c4cccc5oc6ccccc6c45)c4ccc(-c5ccccc5)c5oc6ccccc6c45)c23)cc1 ; c1ccc(-c2ccc3oc4c(-c5ccccc5)cccc4c3c2N(c2cccc3oc4ccccc4c23)c2ccc(-c3ccccc3)c3oc4ccccc4c23)cc1 ; c1ccc(-c2cc(-c3ccccc3)c3c(c2)oc2ccc(N(c4cccc5oc6ccccc6c45)c4ccc(-c5ccccc5)c5oc6ccccc6c45)cc23)cc1]

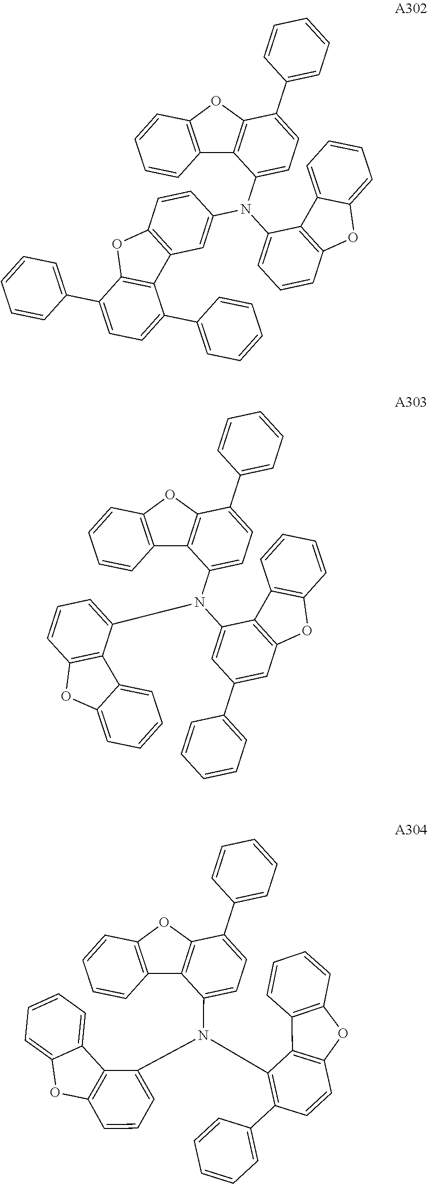 [SMILES: c1ccc(-c2ccc(-c3ccccc3)c3c2oc2ccc(N(c4cccc5oc6ccccc6c45)c4ccc(-c5ccccc5)c5oc6ccccc6c45)cc23)cc1 ; c1ccc(-c2cc(N(c3cccc4oc5ccccc5c34)c3ccc(-c4ccccc4)c4oc5ccccc5c34)c3c(c2)oc2ccccc23)cc1 ; c1ccc(-c2ccc3oc4ccccc4c3c2N(c2cccc3oc4ccccc4c23)c2ccc(-c3ccccc3)c3oc4ccccc4c23)cc1]

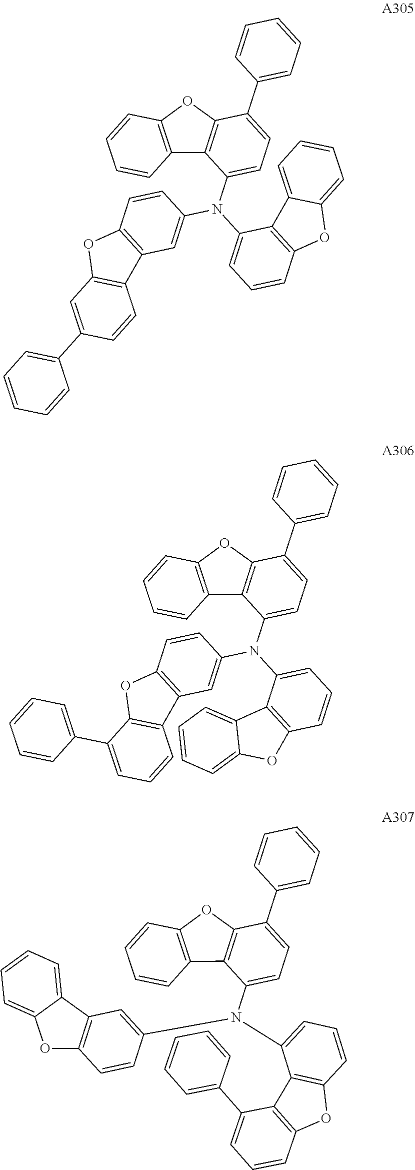 [SMILES: c1ccc(-c2ccc3c(c2)oc2ccc(N(c4cccc5oc6ccccc6c45)c4ccc(-c5ccccc5)c5oc6ccccc6c45)cc23)cc1 ; c1ccc(-c2cccc3c2oc2ccc(N(c4cccc5oc6ccccc6c45)c4ccc(-c5ccccc5)c5oc6ccccc6c45)cc23)cc1 ; c1ccc(-c2ccc(N(c3ccc4oc5ccccc5c4c3)c3cccc4oc5cccc(-c6ccccc6)c5c34)c3c2oc2ccccc23)cc1]

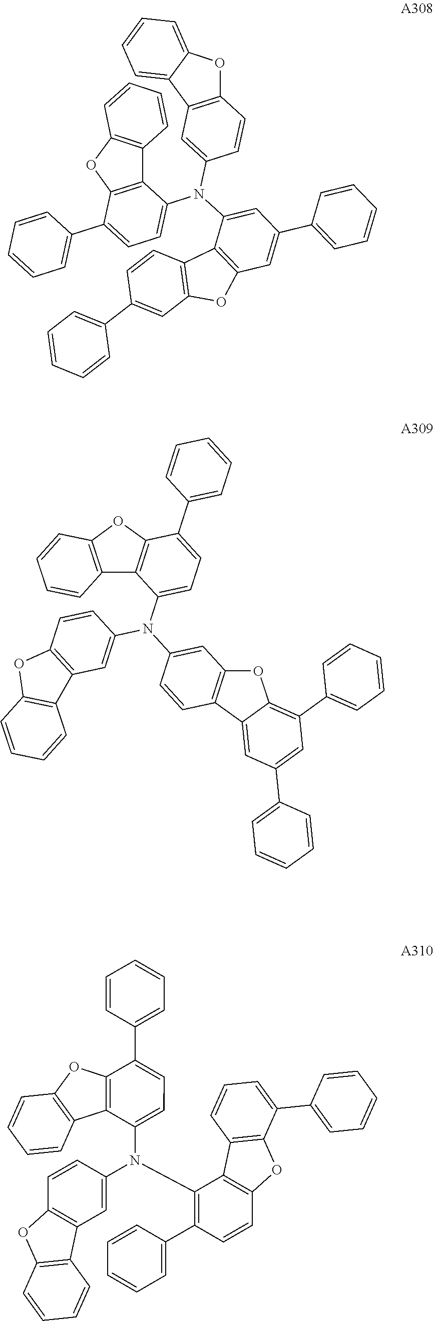 [SMILES: c1ccc(-c2ccc3c(c2)oc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3oc4cc(N(c5ccc6oc7ccccc7c6c5)c5ccc(-c6ccccc6)c6oc7ccccc7c56)ccc4c3c2)cc1 ; c1ccc(-c2ccc3oc4c(-c5ccccc5)cccc4c3c2N(c2ccc3oc4ccccc4c3c2)c2ccc(-c3ccccc3)c3oc4ccccc4c23)cc1]

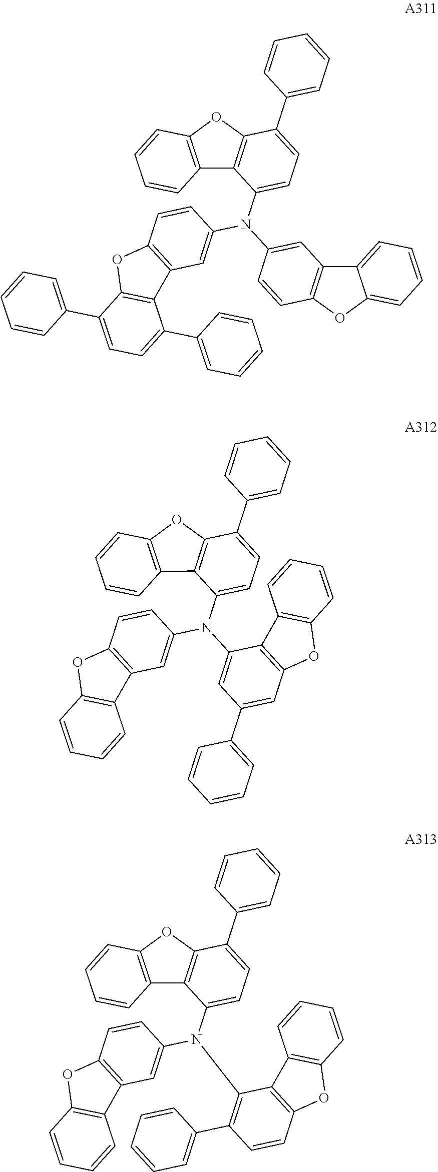 [SMILES: c1ccc(-c2ccc(-c3ccccc3)c3c2oc2ccc(N(c4ccc5oc6ccccc6c5c4)c4ccc(-c5ccccc5)c5oc6ccccc6c45)cc23)cc1 ; c1ccc(-c2cc(N(c3ccc4oc5ccccc5c4c3)c3ccc(-c4ccccc4)c4oc5ccccc5c34)c3c(c2)oc2ccccc23)cc1 ; c1ccc(-c2ccc3oc4ccccc4c3c2N(c2ccc3oc4ccccc4c3c2)c2ccc(-c3ccccc3)c3oc4ccccc4c23)cc1]

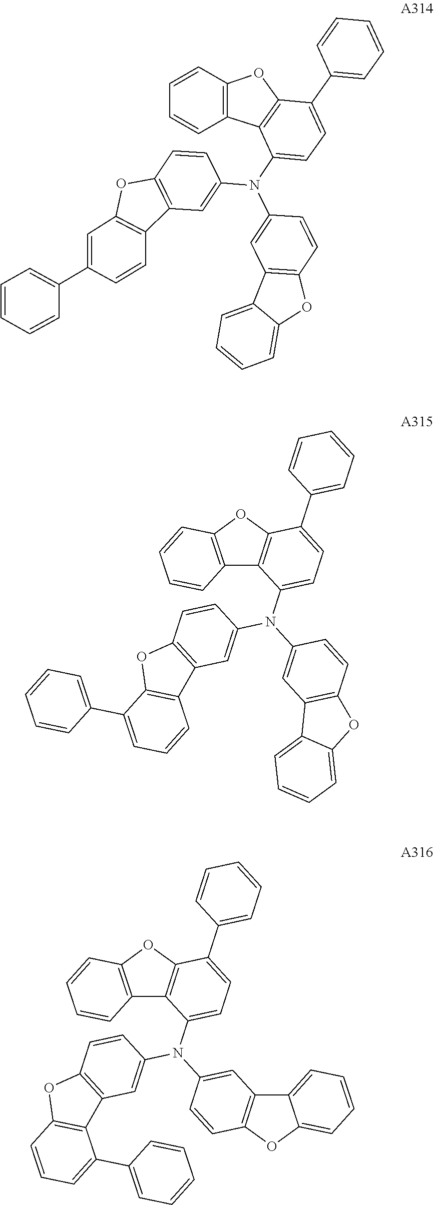 [SMILES: c1ccc(-c2ccc3c(c2)oc2ccc(N(c4ccc5oc6ccccc6c5c4)c4ccc(-c5ccccc5)c5oc6ccccc6c45)cc23)cc1 ; c1ccc(-c2cccc3c2oc2ccc(N(c4ccc5oc6ccccc6c5c4)c4ccc(-c5ccccc5)c5oc6ccccc6c45)cc23)cc1 ; c1ccc(-c2ccc(N(c3ccc4oc5ccccc5c4c3)c3ccc4oc5cccc(-c6ccccc6)c5c4c3)c3c2oc2ccccc23)cc1]

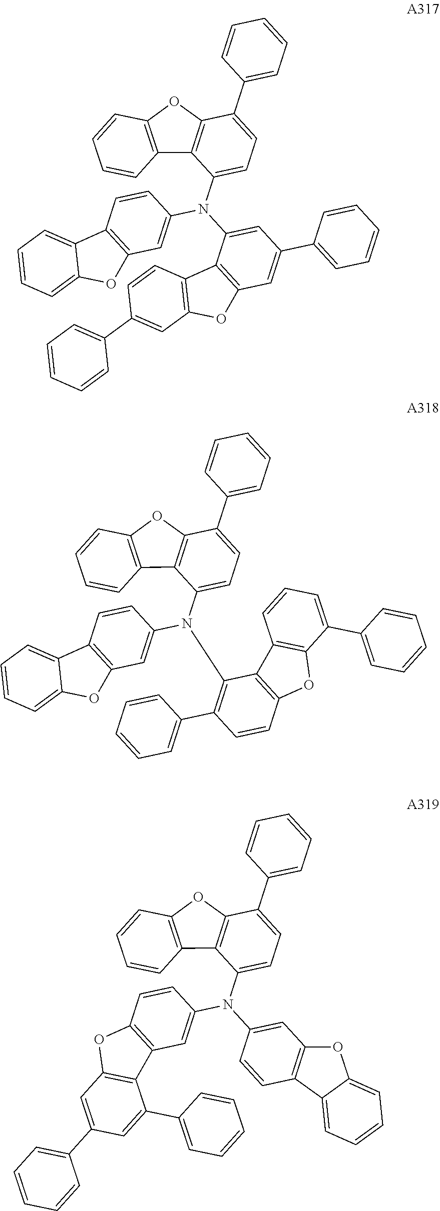 [SMILES: c1ccc(-c2ccc3c(c2)oc2cc(-c4ccccc4)cc(N(c4ccc5c(c4)oc4ccccc45)c4ccc(-c5ccccc5)c5oc6ccccc6c45)c23)cc1 ; c1ccc(-c2ccc3oc4c(-c5ccccc5)cccc4c3c2N(c2ccc3c(c2)oc2ccccc23)c2ccc(-c3ccccc3)c3oc4ccccc4c23)cc1 ; c1ccc(-c2cc(-c3ccccc3)c3c(c2)oc2ccc(N(c4ccc5c(c4)oc4ccccc45)c4ccc(-c5ccccc5)c5oc6ccccc6c45)cc23)cc1]

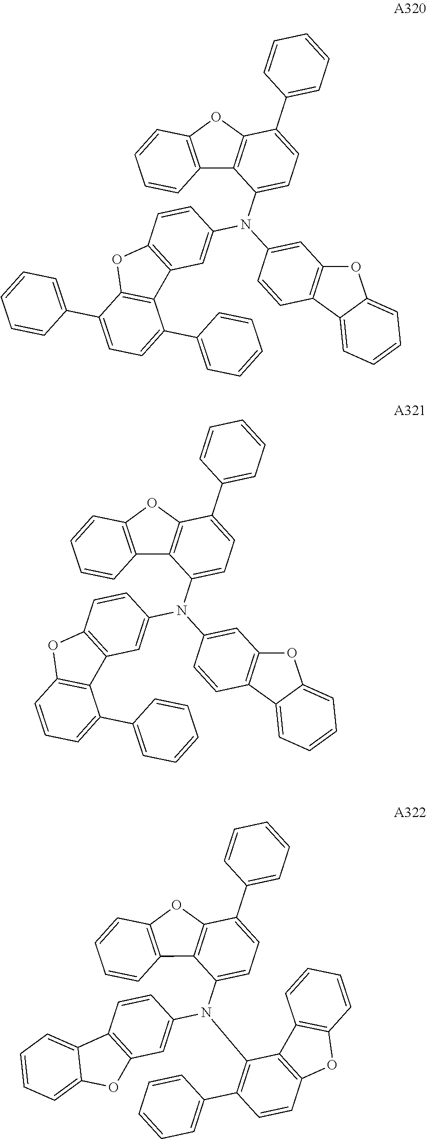

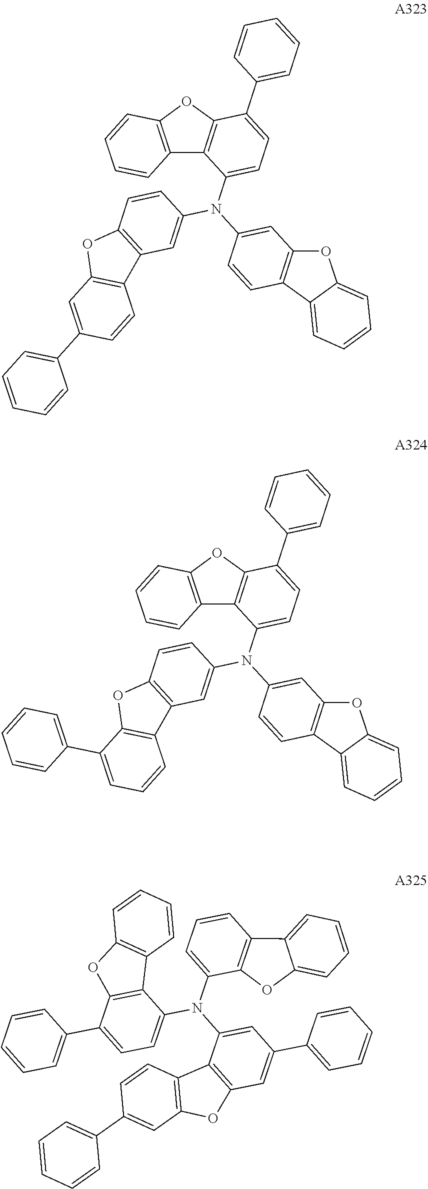 [SMILES: c1ccc(-c2ccc3c(c2)oc2ccc(N(c4ccc5c(c4)oc4ccccc45)c4ccc(-c5ccccc5)c5oc6ccccc6c45)cc23)cc1 ; c1ccc(-c2cccc3c2oc2ccc(N(c4ccc5c(c4)oc4ccccc45)c4ccc(-c5ccccc5)c5oc6ccccc6c45)cc23)cc1 ; c1ccc(-c2ccc3c(c2)oc2cc(-c4ccccc4)cc(N(c4cccc5c4oc4ccccc45)c4ccc(-c5ccccc5)c5oc6ccccc6c45)c23)cc1]

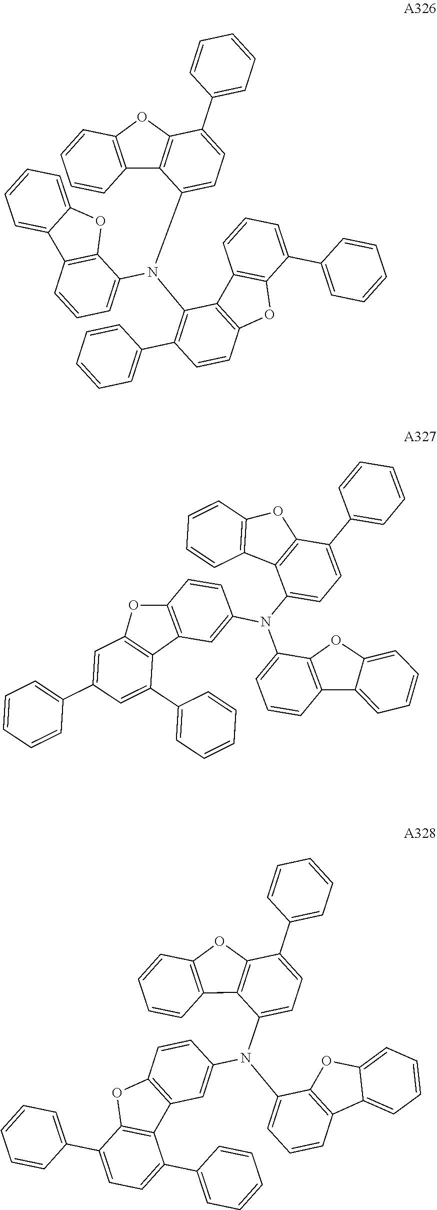 [SMILES: c1ccc(-c2ccc3oc4c(-c5ccccc5)cccc4c3c2N(c2cccc3c2oc2ccccc23)c2ccc(-c3ccccc3)c3oc4ccccc4c23)cc1 ; c1ccc(-c2cc(-c3ccccc3)c3c(c2)oc2ccc(N(c4cccc5c4oc4ccccc45)c4ccc(-c5ccccc5)c5oc6ccccc6c45)cc23)cc1 ; c1ccc(-c2ccc(-c3ccccc3)c3c2oc2ccc(N(c4cccc5c4oc4ccccc45)c4ccc(-c5ccccc5)c5oc6ccccc6c45)cc23)cc1]

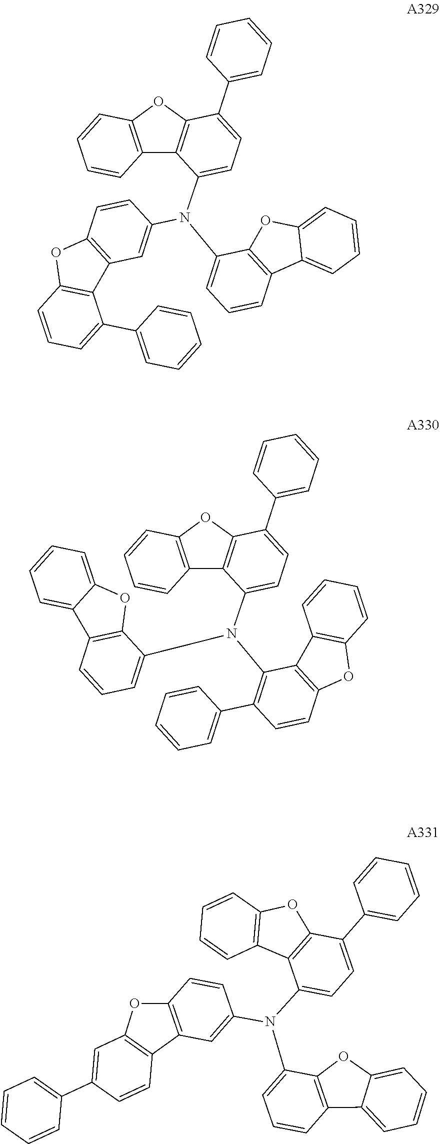 [SMILES: c1ccc(-c2ccc(N(c3ccc4oc5cccc(-c6ccccc6)c5c4c3)c3cccc4c3oc3ccccc34)c3c2oc2ccccc23)cc1 ; c1ccc(-c2ccc3oc4ccccc4c3c2N(c2cccc3c2oc2ccccc23)c2ccc(-c3ccccc3)c3oc4ccccc4c23)cc1 ; c1ccc(-c2ccc3c(c2)oc2ccc(N(c4cccc5c4oc4ccccc45)c4ccc(-c5ccccc5)c5oc6ccccc6c45)cc23)cc1]

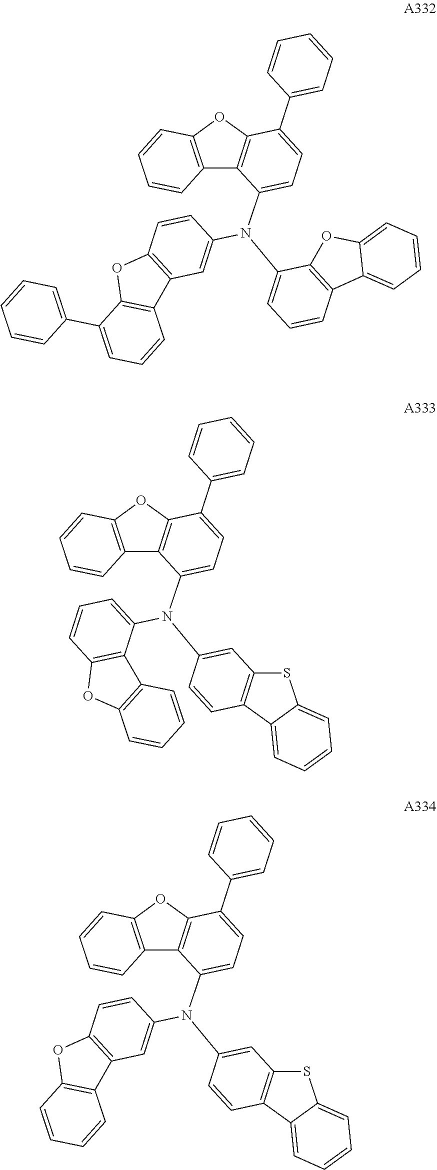 [SMILES: c1ccc(-c2cccc3c2oc2ccc(N(c4cccc5c4oc4ccccc45)c4ccc(-c5ccccc5)c5oc6ccccc6c45)cc23)cc1 ; c1ccc(-c2ccc(N(c3ccc4c(c3)sc3ccccc34)c3cccc4oc5ccccc5c34)c3c2oc2ccccc23)cc1 ; c1ccc(-c2ccc(N(c3ccc4c(c3)sc3ccccc34)c3ccc4oc5ccccc5c4c3)c3c2oc2ccccc23)cc1]

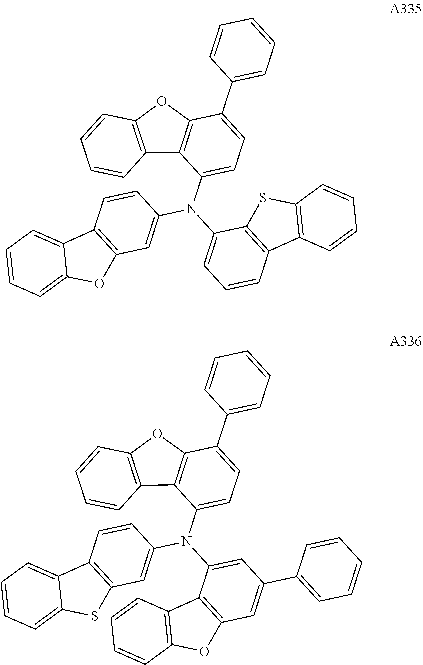 [SMILES: c1ccc(-c2ccc(N(c3ccc4c(c3)oc3ccccc34)c3cccc4c3sc3ccccc34)c3c2oc2ccccc23)cc1 ; c1ccc(-c2cc(N(c3ccc4c(c3)sc3ccccc34)c3ccc(-c4ccccc4)c4oc5ccccc5c34)c3c(c2)oc2ccccc23)cc1]

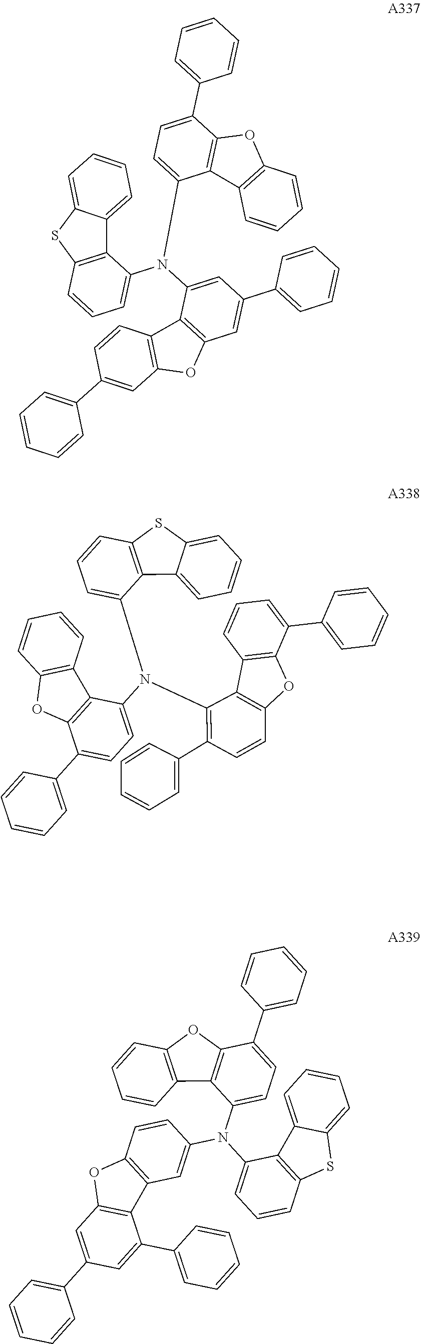 [SMILES: c1ccc(-c2ccc3c(c2)oc2cc(-c4ccccc4)cc(N(c4cccc5sc6ccccc6c45)c4ccc(-c5ccccc5)c5oc6ccccc6c45)c23)cc1 ; c1ccc(-c2ccc3oc4c(-c5ccccc5)cccc4c3c2N(c2cccc3sc4ccccc4c23)c2ccc(-c3ccccc3)c3oc4ccccc4c23)cc1 ; c1ccc(-c2cc(-c3ccccc3)c3c(c2)oc2ccc(N(c4cccc5sc6ccccc6c45)c4ccc(-c5ccccc5)c5oc6ccccc6c45)cc23)cc1]

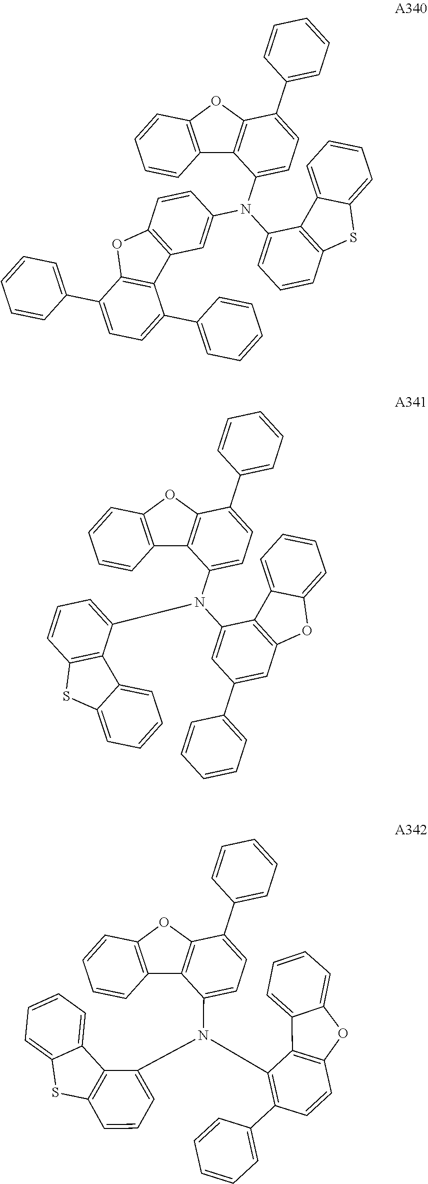 [SMILES: c1ccc(-c2ccc(-c3ccccc3)c3c2oc2ccc(N(c4cccc5sc6ccccc6c45)c4ccc(-c5ccccc5)c5oc6ccccc6c45)cc23)cc1 ; c1ccc(-c2cc(N(c3cccc4sc5ccccc5c34)c3ccc(-c4ccccc4)c4oc5ccccc5c34)c3c(c2)oc2ccccc23)cc1 ; c1ccc(-c2ccc3oc4ccccc4c3c2N(c2cccc3sc4ccccc4c23)c2ccc(-c3ccccc3)c3oc4ccccc4c23)cc1]

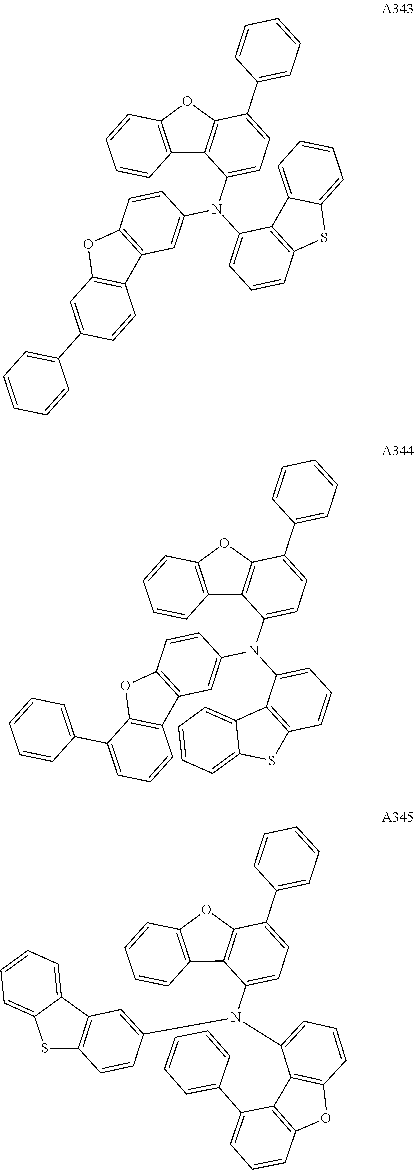 [SMILES: c1ccc(-c2ccc3c(c2)oc2ccc(N(c4cccc5sc6ccccc6c45)c4ccc(-c5ccccc5)c5oc6ccccc6c45)cc23)cc1 ; c1ccc(-c2cccc3c2oc2ccc(N(c4cccc5sc6ccccc6c45)c4ccc(-c5ccccc5)c5oc6ccccc6c45)cc23)cc1 ; c1ccc(-c2ccc(N(c3ccc4sc5ccccc5c4c3)c3cccc4oc5cccc(-c6ccccc6)c5c34)c3c2oc2ccccc23)cc1]

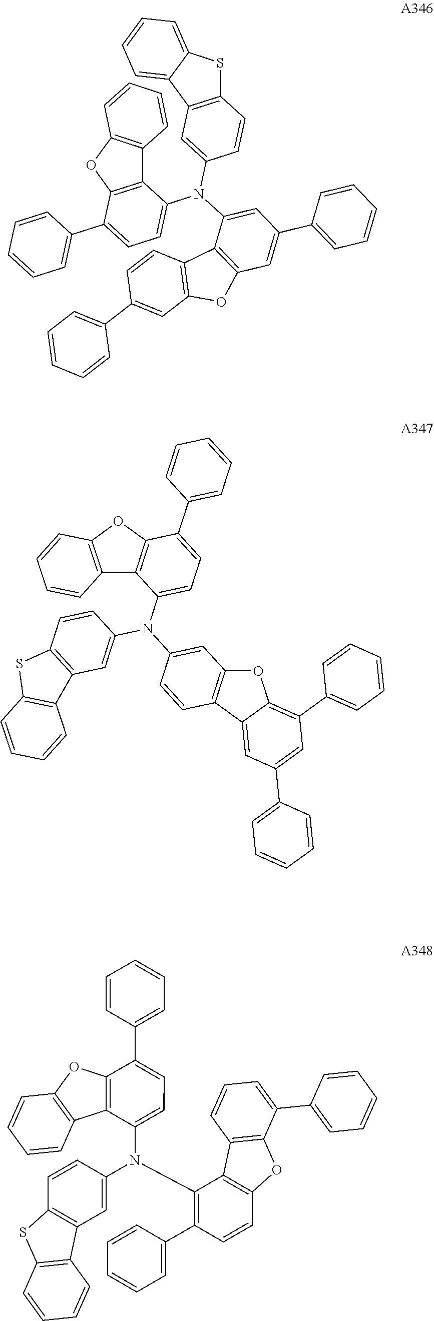 [SMILES: c1ccc(-c2ccc3c(c2)oc2cc(-c4ccccc4)cc(N(c4ccc5sc6ccccc6c5c4)c4ccc(-c5ccccc5)c5oc6ccccc6c45)c23)cc1 ; c1ccc(-c2cc(-c3ccccc3)c3oc4cc(N(c5ccc6sc7ccccc7c6c5)c5ccc(-c6ccccc6)c6oc7ccccc7c56)ccc4c3c2)cc1 ; c1ccc(-c2ccc3oc4c(-c5ccccc5)cccc4c3c2N(c2ccc3sc4ccccc4c3c2)c2ccc(-c3ccccc3)c3oc4ccccc4c23)cc1]

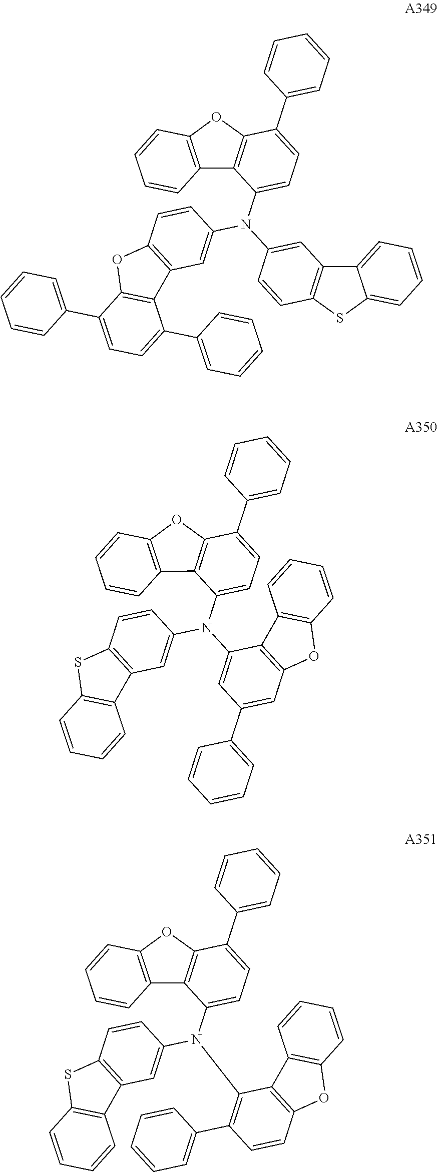 [SMILES: c1ccc(-c2ccc(-c3ccccc3)c3c2oc2ccc(N(c4ccc5sc6ccccc6c5c4)c4ccc(-c5ccccc5)c5oc6ccccc6c45)cc23)cc1 ; c1ccc(-c2cc(N(c3ccc4sc5ccccc5c4c3)c3ccc(-c4ccccc4)c4oc5ccccc5c34)c3c(c2)oc2ccccc23)cc1 ; c1ccc(-c2ccc3oc4ccccc4c3c2N(c2ccc3sc4ccccc4c3c2)c2ccc(-c3ccccc3)c3oc4ccccc4c23)cc1]

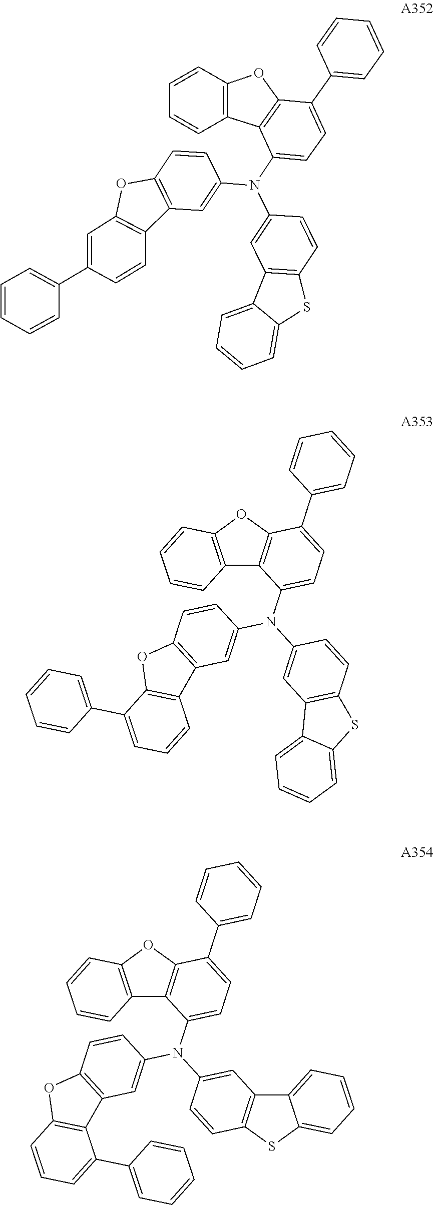 [SMILES: c1ccc(-c2ccc3c(c2)oc2ccc(N(c4ccc5sc6ccccc6c5c4)c4ccc(-c5ccccc5)c5oc6ccccc6c45)cc23)cc1 ; c1ccc(-c2cccc3c2oc2ccc(N(c4ccc5sc6ccccc6c5c4)c4ccc(-c5ccccc5)c5oc6ccccc6c45)cc23)cc1 ; c1ccc(-c2ccc(N(c3ccc4sc5ccccc5c4c3)c3ccc4oc5cccc(-c6ccccc6)c5c4c3)c3c2oc2ccccc23)cc1]

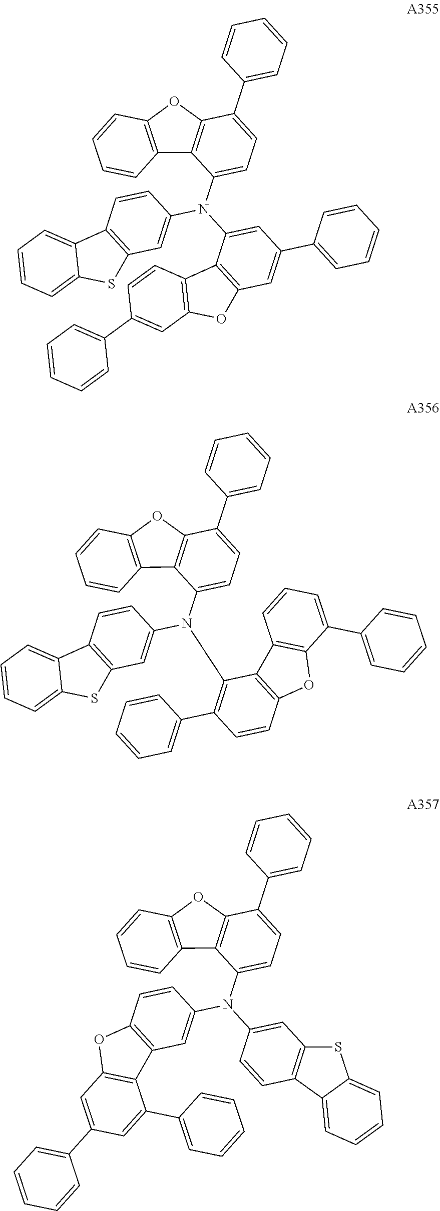 [SMILES: c1ccc(-c2ccc3c(c2)oc2cc(-c4ccccc4)cc(N(c4ccc5c(c4)sc4ccccc45)c4ccc(-c5ccccc5)c5oc6ccccc6c45)c23)cc1 ; c1ccc(-c2ccc3oc4c(-c5ccccc5)cccc4c3c2N(c2ccc3c(c2)sc2ccccc23)c2ccc(-c3ccccc3)c3oc4ccccc4c23)cc1 ; c1ccc(-c2cc(-c3ccccc3)c3c(c2)oc2ccc(N(c4ccc5c(c4)sc4ccccc45)c4ccc(-c5ccccc5)c5oc6ccccc6c45)cc23)cc1]

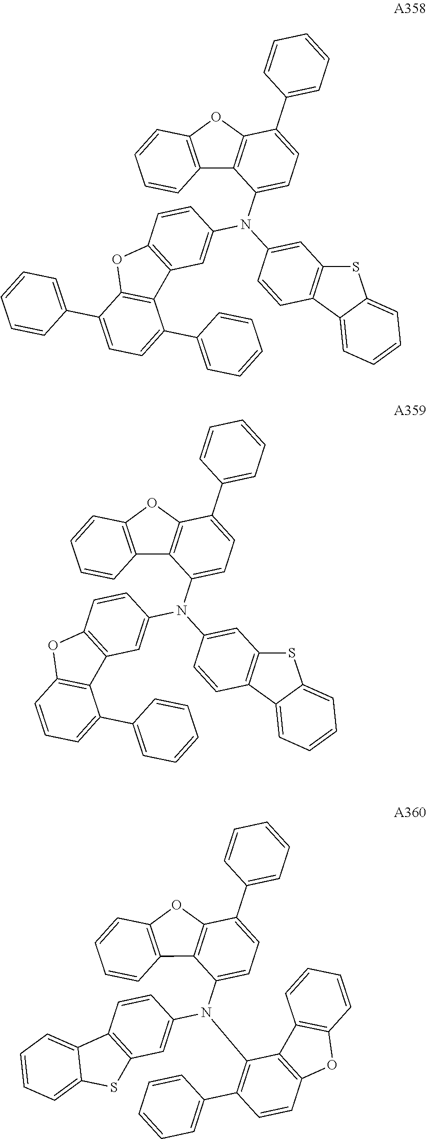

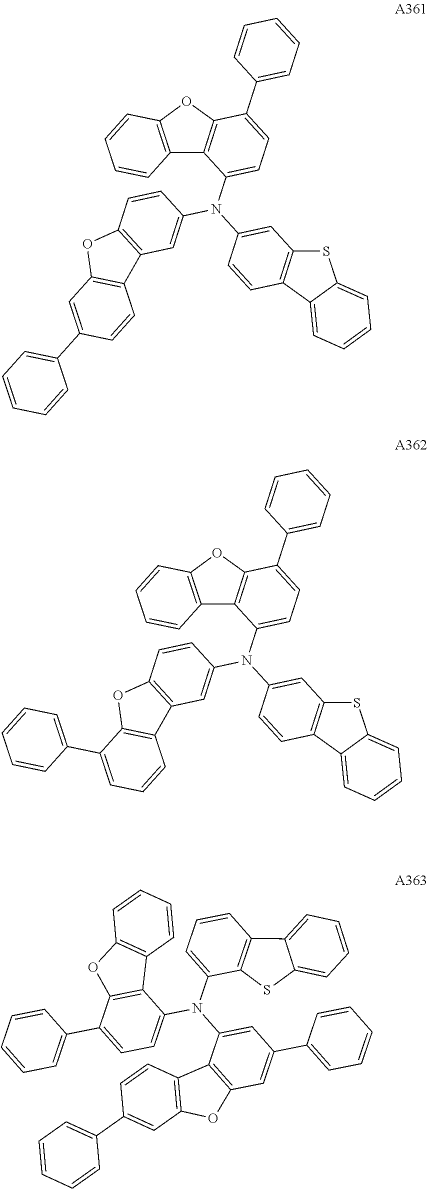 [SMILES: c1ccc(-c2ccc3c(c2)oc2ccc(N(c4ccc5c(c4)sc4ccccc45)c4ccc(-c5ccccc5)c5oc6ccccc6c45)cc23)cc1 ; c1ccc(-c2cccc3c2oc2ccc(N(c4ccc5c(c4)sc4ccccc45)c4ccc(-c5ccccc5)c5oc6ccccc6c45)cc23)cc1 ; c1ccc(-c2ccc3c(c2)oc2cc(-c4ccccc4)cc(N(c4cccc5c4sc4ccccc45)c4ccc(-c5ccccc5)c5oc6ccccc6c45)c23)cc1]

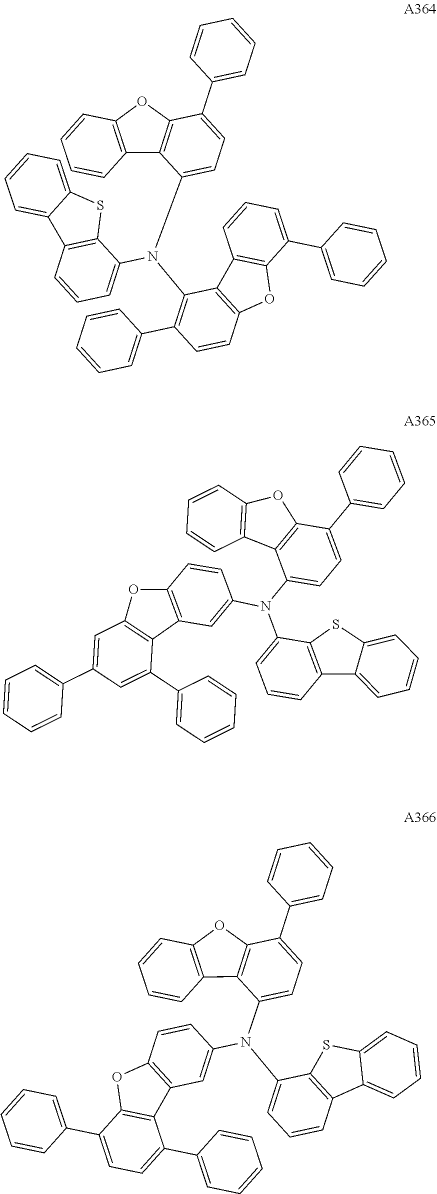 [SMILES: c1ccc(-c2ccc3oc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)oc2ccc(N(c4cccc5c4sc4ccccc45)c4ccc(-c5ccccc5)c5oc6ccccc6c45)cc23)cc1 ; c1ccc(-c2ccc(-c3ccccc3)c3c2oc2ccc(N(c4cccc5c4sc4ccccc45)c4ccc(-c5ccccc5)c5oc6ccccc6c45)cc23)cc1]

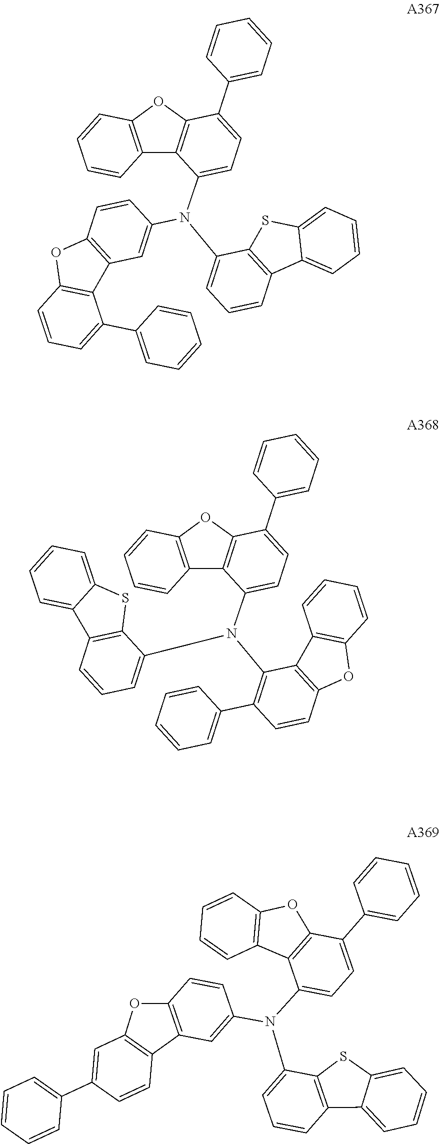 [SMILES: c1ccc(-c2ccc(N(c3ccc4oc5cccc(-c6ccccc6)c5c4c3)c3cccc4c3sc3ccccc34)c3c2oc2ccccc23)cc1 ; c1ccc(-c2ccc3oc4ccccc4c3c2N(c2cccc3c2sc2ccccc23)c2ccc(-c3ccccc3)c3oc4ccccc4c23)cc1 ; c1ccc(-c2ccc3c(c2)oc2ccc(N(c4cccc5c4sc4ccccc45)c4ccc(-c5ccccc5)c5oc6ccccc6c45)cc23)cc1]

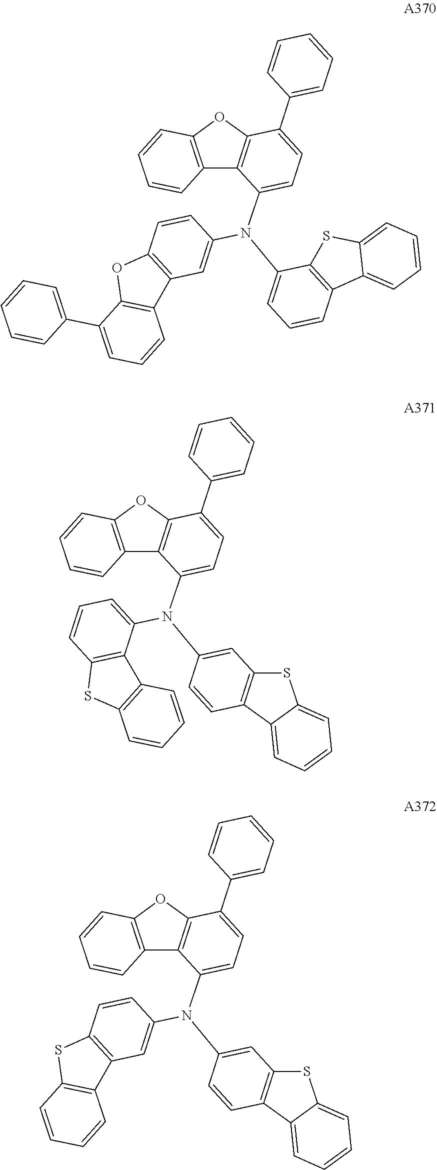 [SMILES: c1ccc(-c2cccc3c2oc2ccc(N(c4cccc5c4sc4ccccc45)c4ccc(-c5ccccc5)c5oc6ccccc6c45)cc23)cc1 ; c1ccc(-c2ccc(N(c3ccc4c(c3)sc3ccccc34)c3cccc4sc5ccccc5c34)c3c2oc2ccccc23)cc1 ; c1ccc(-c2ccc(N(c3ccc4c(c3)sc3ccccc34)c3ccc4sc5ccccc5c4c3)c3c2oc2ccccc23)cc1]

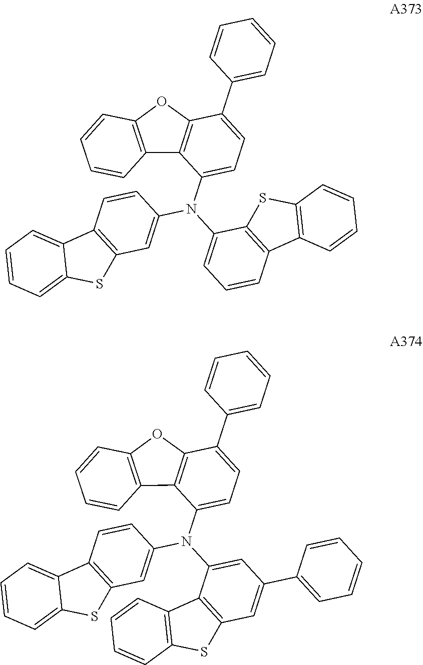 [SMILES: c1ccc(-c2ccc(N(c3ccc4c(c3)sc3ccccc34)c3cccc4c3sc3ccccc34)c3c2oc2ccccc23)cc1 ; c1ccc(-c2cc(N(c3ccc4c(c3)sc3ccccc34)c3ccc(-c4ccccc4)c4oc5ccccc5c34)c3c(c2)sc2ccccc23)cc1]

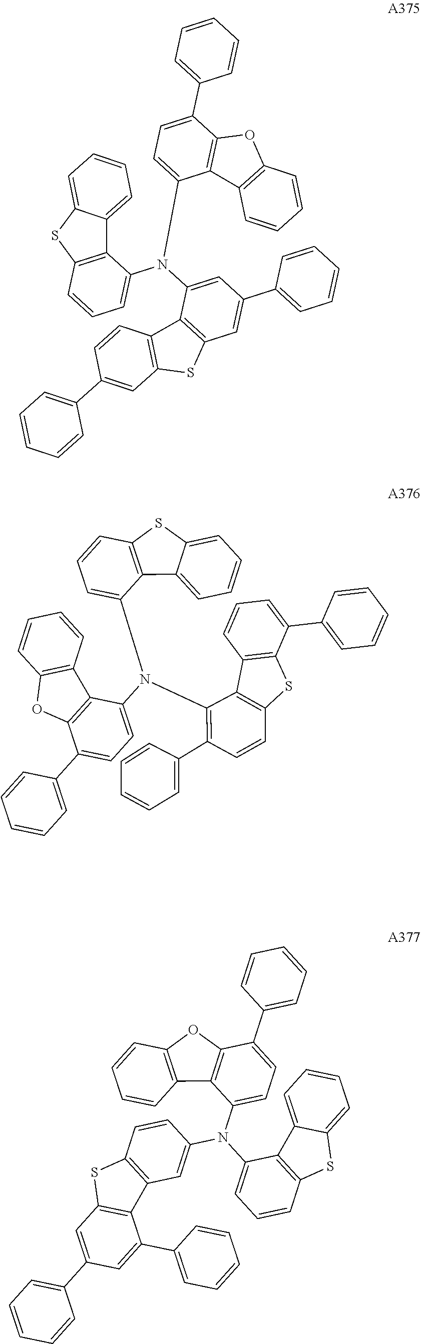 [SMILES: c1ccc(-c2ccc3c(c2)sc2cc(-c4ccccc4)cc(N(c4cccc5sc6ccccc6c45)c4ccc(-c5ccccc5)c5oc6ccccc6c45)c23)cc1 ; c1ccc(-c2ccc3sc4c(-c5ccccc5)cccc4c3c2N(c2cccc3sc4ccccc4c23)c2ccc(-c3ccccc3)c3oc4ccccc4c23)cc1 ; c1ccc(-c2cc(-c3ccccc3)c3c(c2)sc2ccc(N(c4cccc5sc6ccccc6c45)c4ccc(-c5ccccc5)c5oc6ccccc6c45)cc23)cc1]

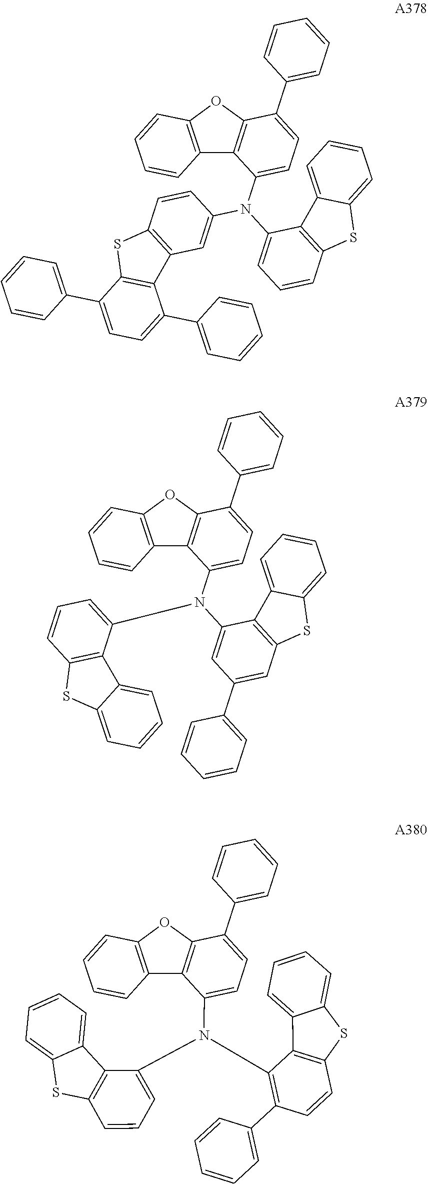 [SMILES: c1ccc(-c2ccc(N(c3ccc4sc5c(-c6ccccc6)ccc(-c6ccccc6)c5c4c3)c3cccc4sc5ccccc5c34)c3c2oc2ccccc23)cc1 ; c1ccc(-c2cc(N(c3cccc4sc5ccccc5c34)c3ccc(-c4ccccc4)c4oc5ccccc5c34)c3c(c2)sc2ccccc23)cc1 ; c1ccc(-c2ccc3sc4ccccc4c3c2N(c2cccc3sc4ccccc4c23)c2ccc(-c3ccccc3)c3oc4ccccc4c23)cc1]

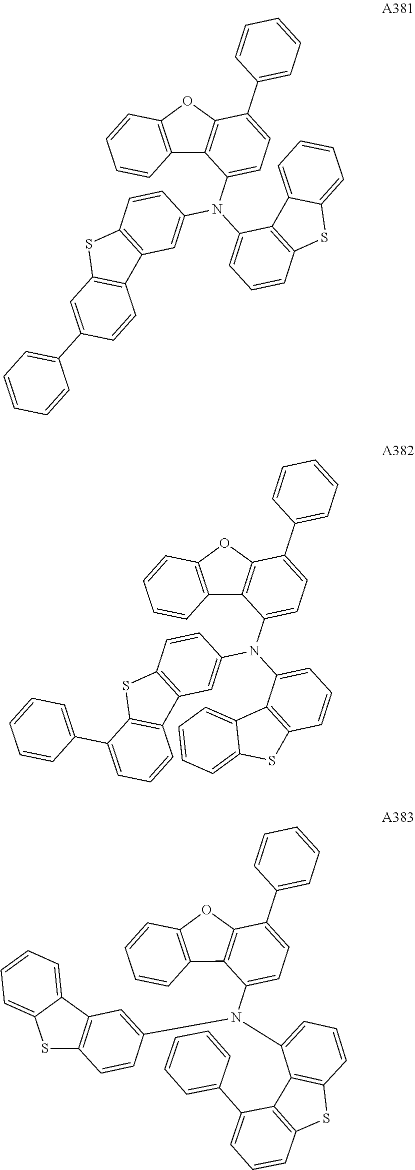 [SMILES: c1ccc(-c2ccc3c(c2)sc2ccc(N(c4cccc5sc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4sc5ccccc5c34)c3c2oc2ccccc23)cc1 ; c1ccc(-c2ccc(N(c3ccc4sc5ccccc5c4c3)c3cccc4sc5cccc(-c6ccccc6)c5c34)c3c2oc2ccccc23)cc1]

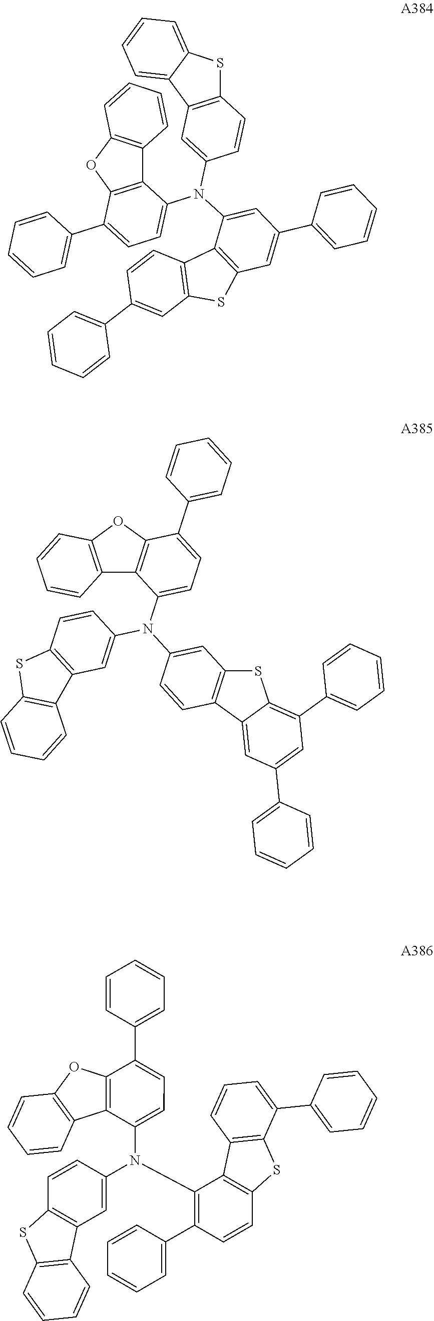 [SMILES: c1ccc(-c2ccc3c(c2)sc2cc(-c4ccccc4)cc(N(c4ccc5sc6ccccc6c5c4)c4ccc(-c5ccccc5)c5oc6ccccc6c45)c23)cc1 ; c1ccc(-c2cc(-c3ccccc3)c3sc4cc(N(c5ccc6sc7ccccc7c6c5)c5ccc(-c6ccccc6)c6oc7ccccc7c56)ccc4c3c2)cc1 ; c1ccc(-c2ccc3sc4c(-c5ccccc5)cccc4c3c2N(c2ccc3sc4ccccc4c3c2)c2ccc(-c3ccccc3)c3oc4ccccc4c23)cc1]

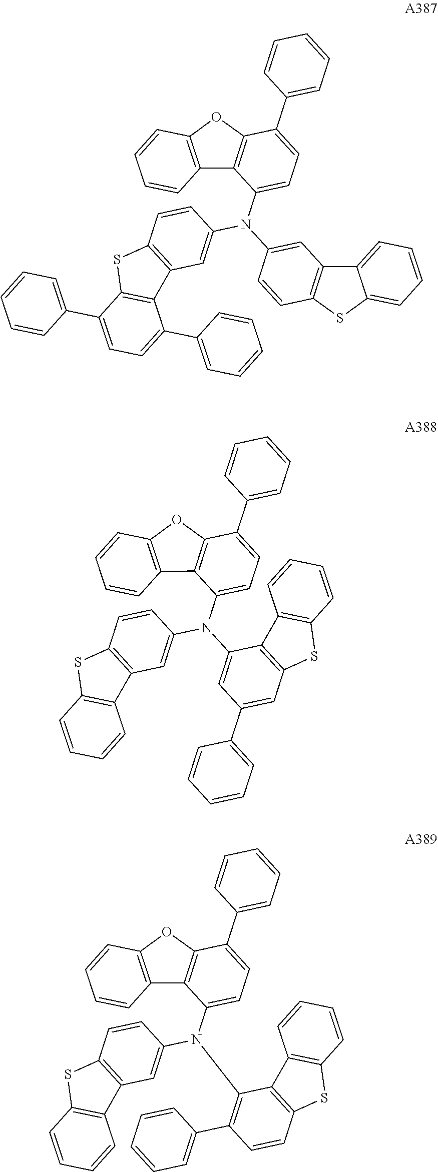 [SMILES: c1ccc(-c2ccc(N(c3ccc4sc5ccccc5c4c3)c3ccc4sc5c(-c6ccccc6)ccc(-c6ccccc6)c5c4c3)c3c2oc2ccccc23)cc1 ; c1ccc(-c2cc(N(c3ccc4sc5ccccc5c4c3)c3ccc(-c4ccccc4)c4oc5ccccc5c34)c3c(c2)sc2ccccc23)cc1 ; c1ccc(-c2ccc3sc4ccccc4c3c2N(c2ccc3sc4ccccc4c3c2)c2ccc(-c3ccccc3)c3oc4ccccc4c23)cc1]

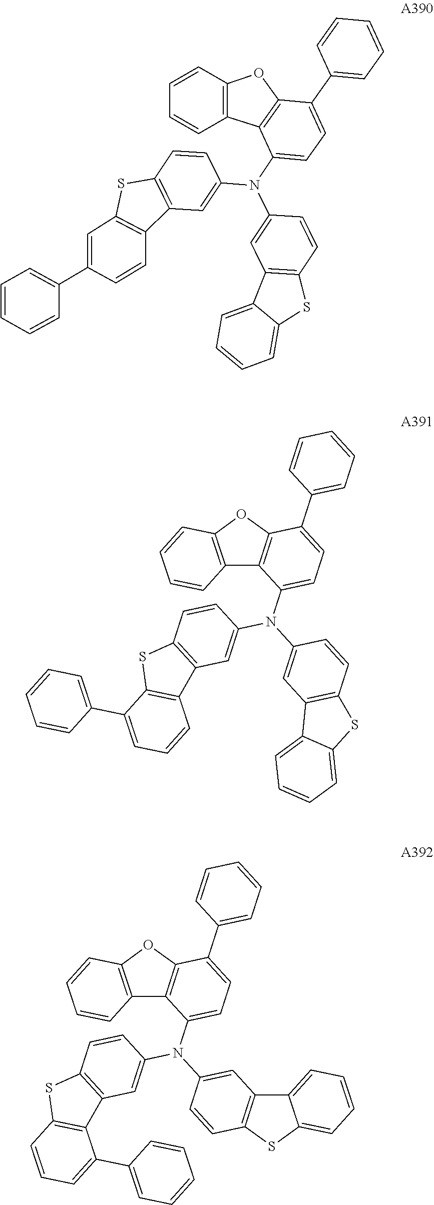 [SMILES: c1ccc(-c2ccc3c(c2)sc2ccc(N(c4ccc5sc6ccccc6c5c4)c4ccc(-c5ccccc5)c5oc6ccccc6c45)cc23)cc1 ; c1ccc(-c2ccc(N(c3ccc4sc5ccccc5c4c3)c3ccc4sc5c(-c6ccccc6)cccc5c4c3)c3c2oc2ccccc23)cc1 ; c1ccc(-c2ccc(N(c3ccc4sc5ccccc5c4c3)c3ccc4sc5cccc(-c6ccccc6)c5c4c3)c3c2oc2ccccc23)cc1]

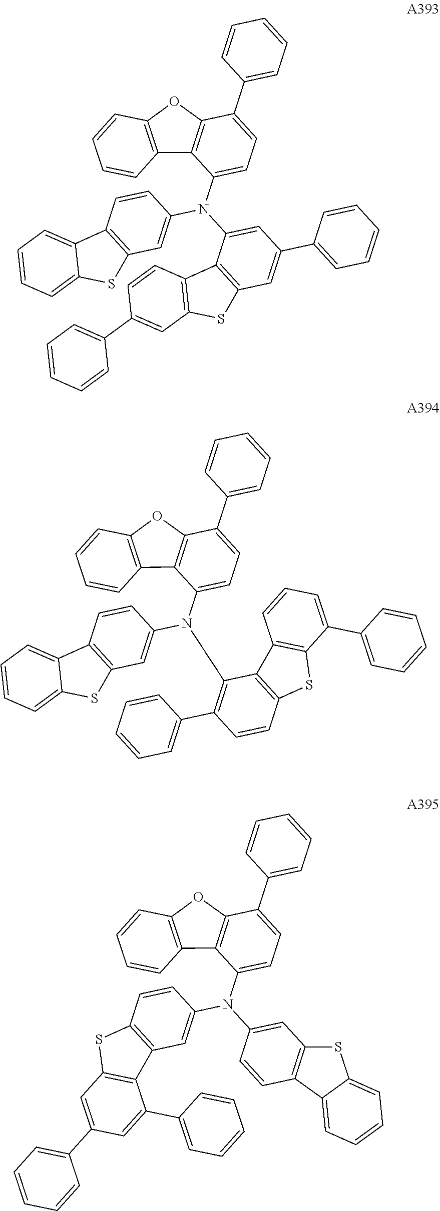 [SMILES: c1ccc(-c2ccc3c(c2)sc2cc(-c4ccccc4)cc(N(c4ccc5c(c4)sc4ccccc45)c4ccc(-c5ccccc5)c5oc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3oc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5oc6ccccc6c45)cc23)cc1]

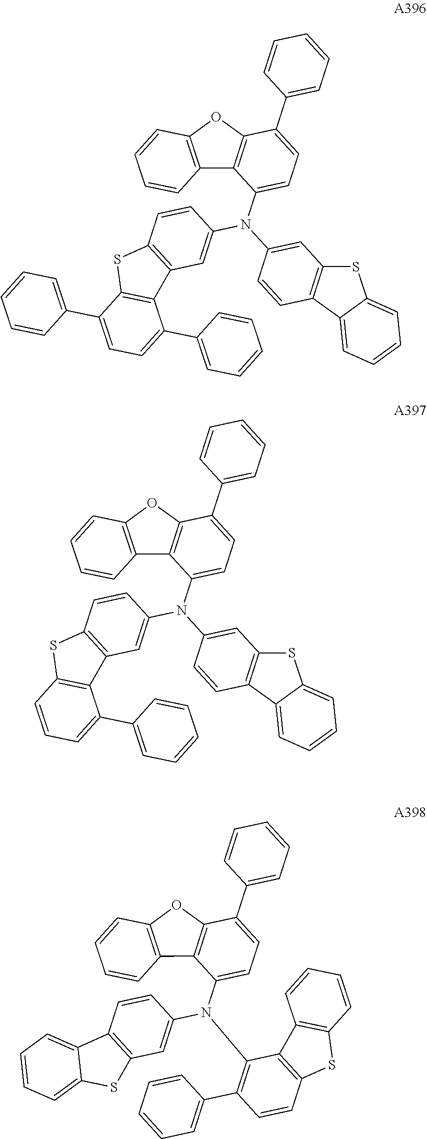

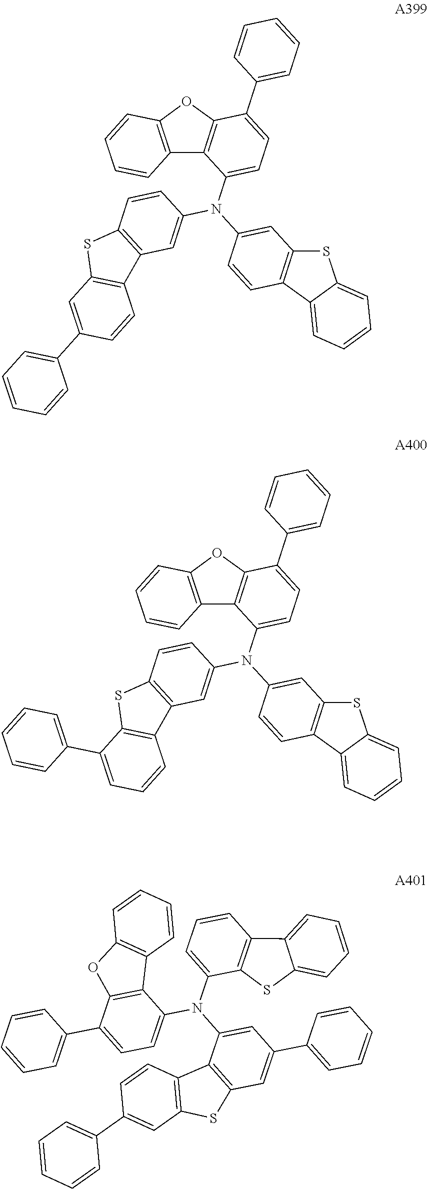 [SMILES: c1ccc(-c2ccc3c(c2)sc2ccc(N(c4ccc5c(c4)sc4ccccc45)c4ccc(-c5ccccc5)c5oc6ccccc6c45)cc23)cc1 ; c1ccc(-c2ccc(N(c3ccc4c(c3)sc3ccccc34)c3ccc4sc5c(-c6ccccc6)cccc5c4c3)c3c2oc2ccccc23)cc1 ; c1ccc(-c2ccc3c(c2)sc2cc(-c4ccccc4)cc(N(c4cccc5c4sc4ccccc45)c4ccc(-c5ccccc5)c5oc6ccccc6c45)c23)cc1]

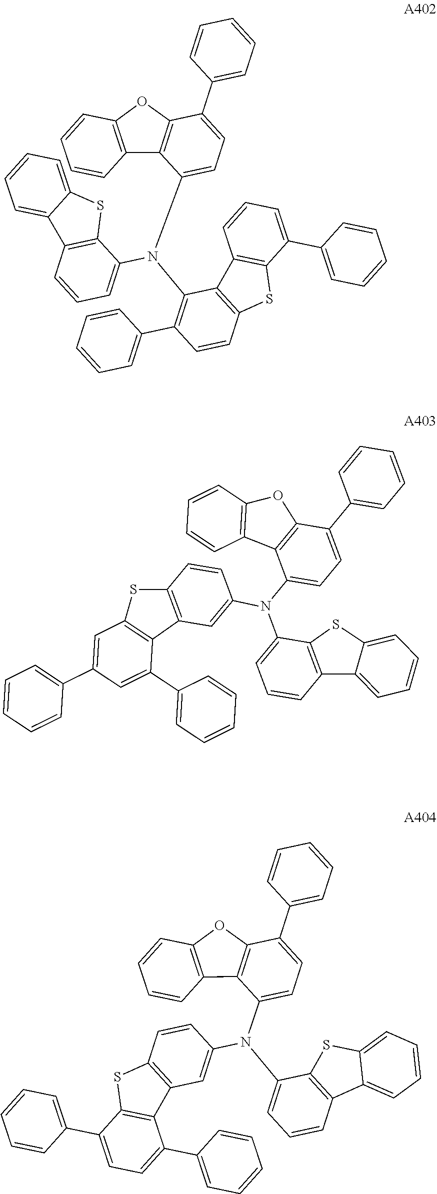 [SMILES: c1ccc(-c2ccc3sc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)sc2ccc(N(c4cccc5c4sc4ccccc45)c4ccc(-c5ccccc5)c5oc6ccccc6c45)cc23)cc1 ; c1ccc(-c2ccc(N(c3ccc4sc5c(-c6ccccc6)ccc(-c6ccccc6)c5c4c3)c3cccc4c3sc3ccccc34)c3c2oc2ccccc23)cc1]

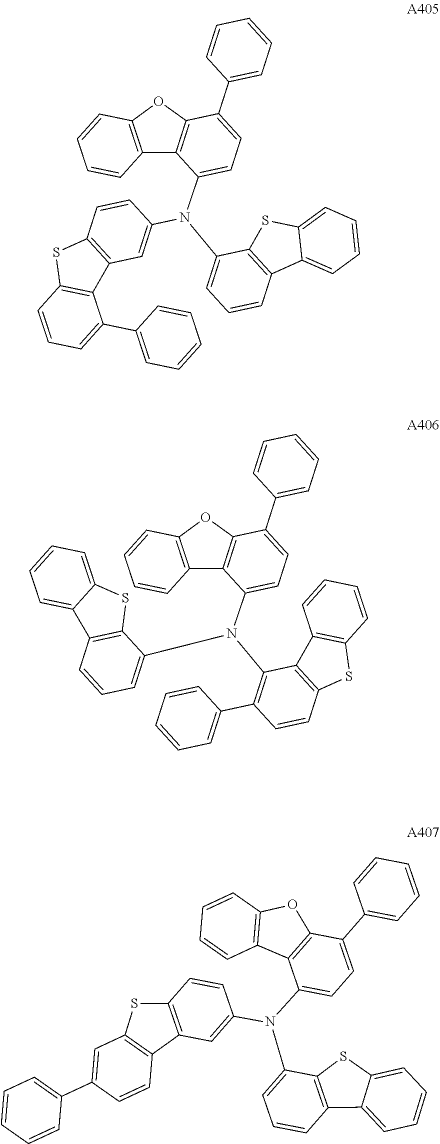 [SMILES: c1ccc(-c2ccc(N(c3ccc4sc5cccc(-c6ccccc6)c5c4c3)c3cccc4c3sc3ccccc34)c3c2oc2ccccc23)cc1 ; c1ccc(-c2ccc3sc4ccccc4c3c2N(c2cccc3c2sc2ccccc23)c2ccc(-c3ccccc3)c3oc4ccccc4c23)cc1 ; c1ccc(-c2ccc3c(c2)sc2ccc(N(c4cccc5c4sc4ccccc45)c4ccc(-c5ccccc5)c5oc6ccccc6c45)cc23)cc1]

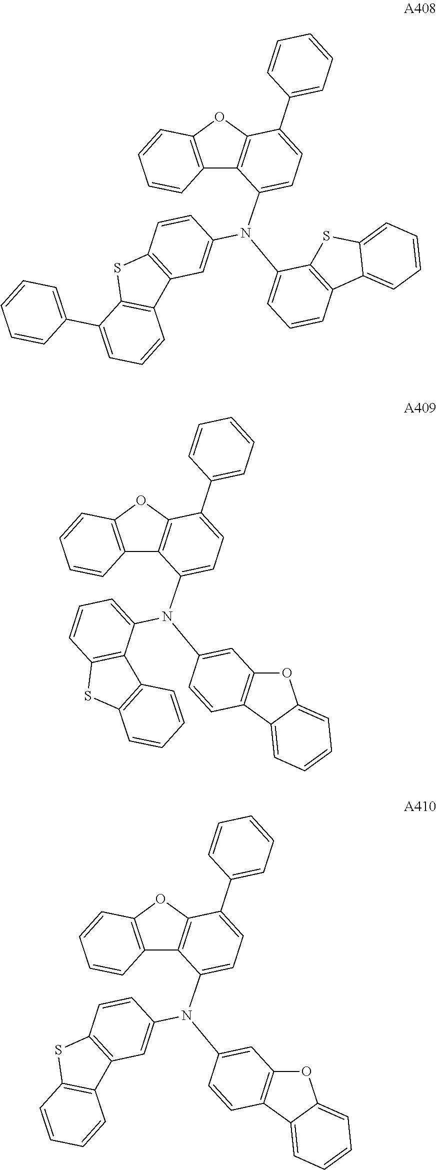 [SMILES: c1ccc(-c2ccc(N(c3ccc4sc5c(-c6ccccc6)cccc5c4c3)c3cccc4c3sc3ccccc34)c3c2oc2ccccc23)cc1 ; c1ccc(-c2ccc(N(c3ccc4c(c3)oc3ccccc34)c3cccc4sc5ccccc5c34)c3c2oc2ccccc23)cc1 ; c1ccc(-c2ccc(N(c3ccc4c(c3)oc3ccccc34)c3ccc4sc5ccccc5c4c3)c3c2oc2ccccc23)cc1]

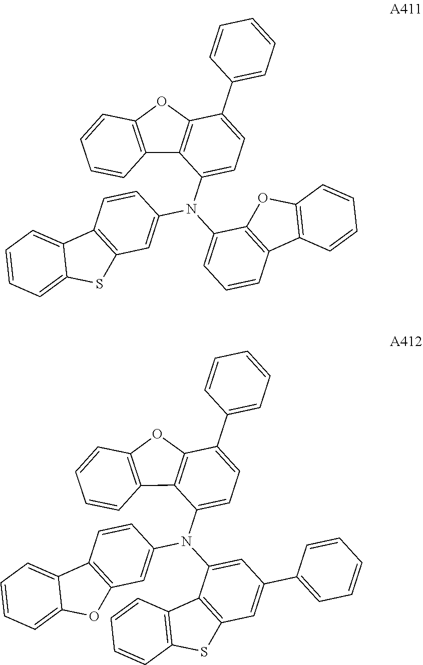 [SMILES: c1ccc(-c2ccc(N(c3ccc4c(c3)sc3ccccc34)c3cccc4c3oc3ccccc34)c3c2oc2ccccc23)cc1 ; c1ccc(-c2cc(N(c3ccc4c(c3)oc3ccccc34)c3ccc(-c4ccccc4)c4oc5ccccc5c34)c3c(c2)sc2ccccc23)cc1]

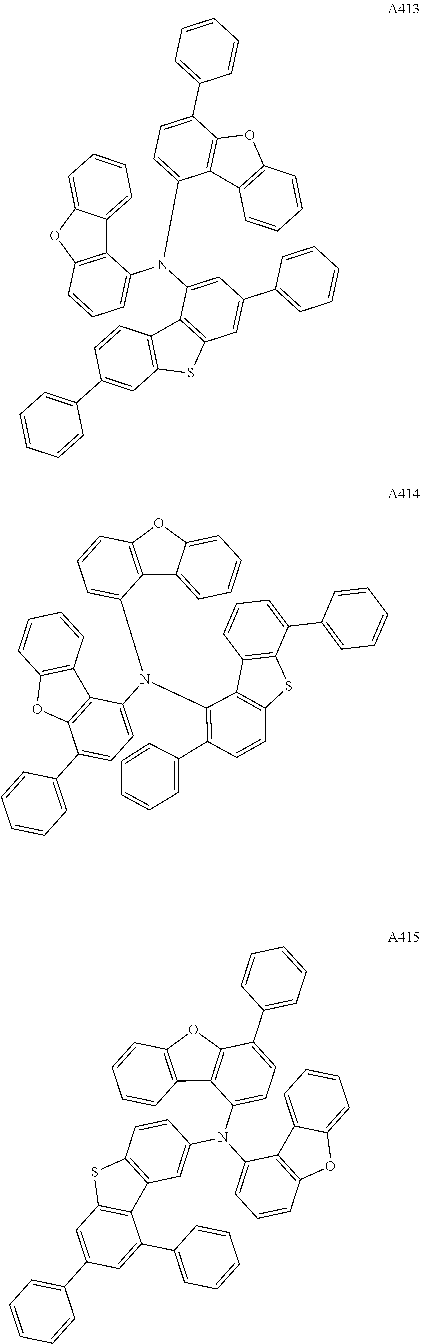 [SMILES: c1ccc(-c2ccc3c(c2)sc2cc(-c4ccccc4)cc(N(c4cccc5oc6ccccc6c45)c4ccc(-c5ccccc5)c5oc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3oc4ccccc4c23)cc1 ; c1ccc(-c2cc(-c3ccccc3)c3c(c2)sc2ccc(N(c4cccc5oc6ccccc6c45)c4ccc(-c5ccccc5)c5oc6ccccc6c45)cc23)cc1]

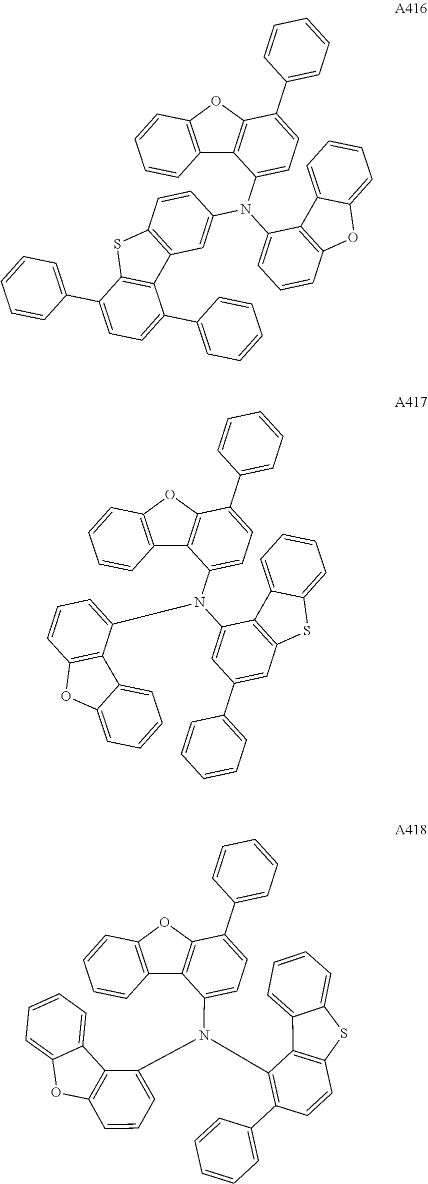 [SMILES: c1ccc(-c2ccc(N(c3ccc4sc5c(-c6ccccc6)ccc(-c6ccccc6)c5c4c3)c3cccc4oc5ccccc5c34)c3c2oc2ccccc23)cc1 ; c1ccc(-c2cc(N(c3cccc4oc5ccccc5c34)c3ccc(-c4ccccc4)c4oc5ccccc5c34)c3c(c2)sc2ccccc23)cc1 ; c1ccc(-c2ccc3sc4ccccc4c3c2N(c2cccc3oc4ccccc4c23)c2ccc(-c3ccccc3)c3oc4ccccc4c23)cc1]

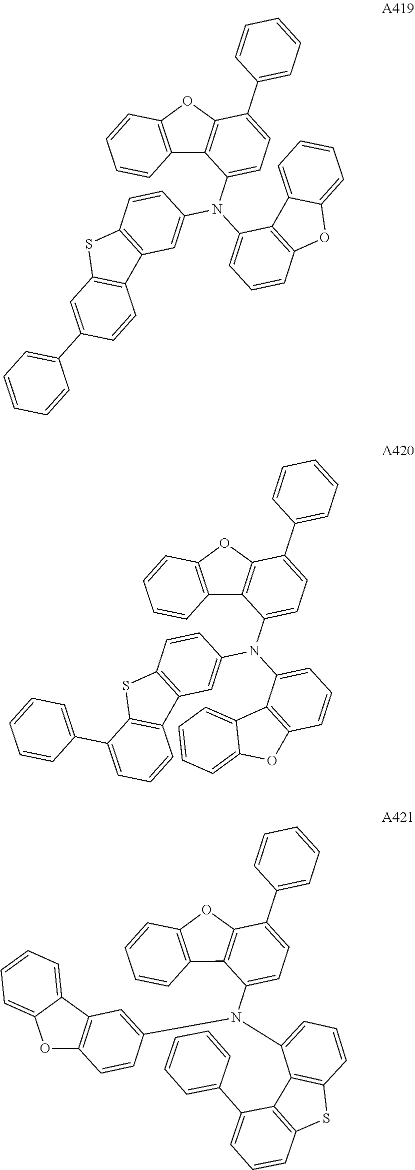 [SMILES: c1ccc(-c2ccc3c(c2)sc2ccc(N(c4cccc5oc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4oc5ccccc5c34)c3c2oc2ccccc23)cc1 ; c1ccc(-c2ccc(N(c3ccc4oc5ccccc5c4c3)c3cccc4sc5cccc(-c6ccccc6)c5c34)c3c2oc2ccccc23)cc1]

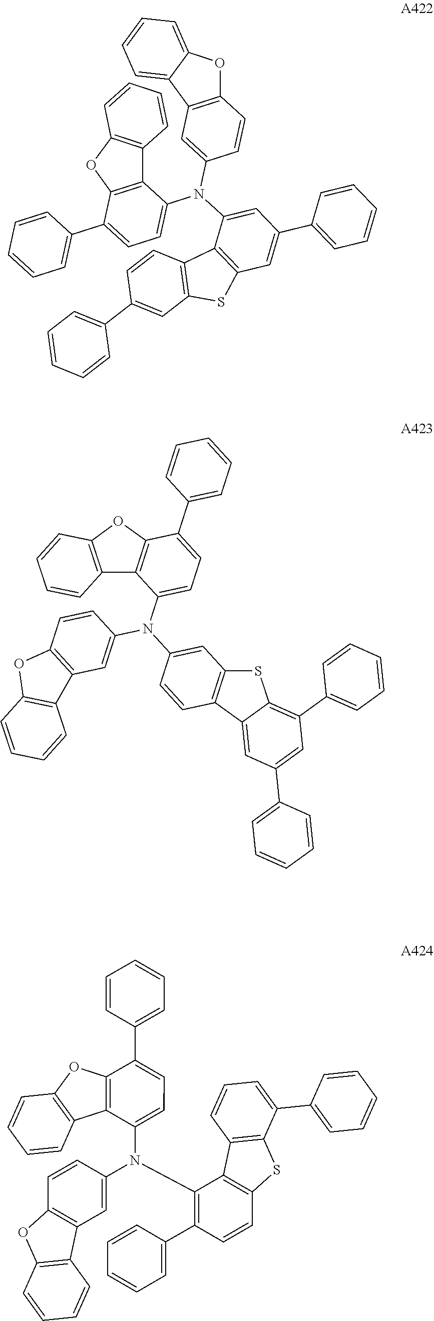 [SMILES: c1ccc(-c2ccc3c(c2)sc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3sc4cc(N(c5ccc6oc7ccccc7c6c5)c5ccc(-c6ccccc6)c6oc7ccccc7c56)ccc4c3c2)cc1 ; c1ccc(-c2ccc3sc4c(-c5ccccc5)cccc4c3c2N(c2ccc3oc4ccccc4c3c2)c2ccc(-c3ccccc3)c3oc4ccccc4c23)cc1]

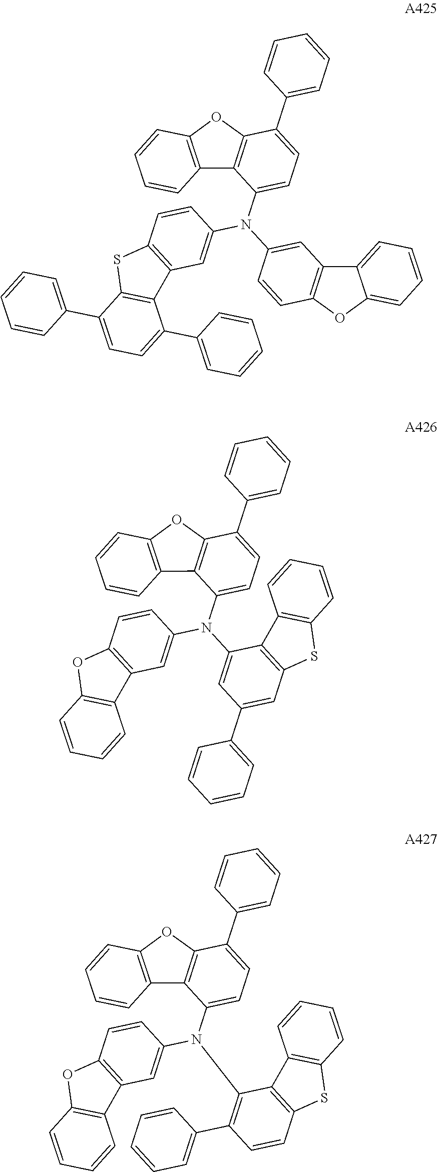 [SMILES: c1ccc(-c2ccc(N(c3ccc4oc5ccccc5c4c3)c3ccc4sc5c(-c6ccccc6)ccc(-c6ccccc6)c5c4c3)c3c2oc2ccccc23)cc1 ; c1ccc(-c2cc(N(c3ccc4oc5ccccc5c4c3)c3ccc(-c4ccccc4)c4oc5ccccc5c34)c3c(c2)sc2ccccc23)cc1 ; c1ccc(-c2ccc3sc4ccccc4c3c2N(c2ccc3oc4ccccc4c3c2)c2ccc(-c3ccccc3)c3oc4ccccc4c23)cc1]

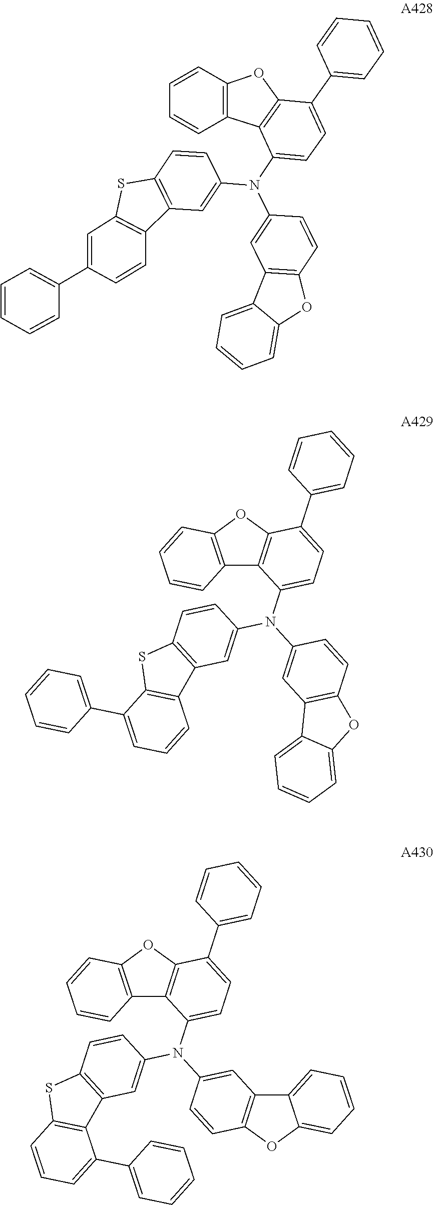 [SMILES: c1ccc(-c2ccc3c(c2)sc2ccc(N(c4ccc5oc6ccccc6c5c4)c4ccc(-c5ccccc5)c5oc6ccccc6c45)cc23)cc1 ; c1ccc(-c2ccc(N(c3ccc4oc5ccccc5c4c3)c3ccc4sc5c(-c6ccccc6)cccc5c4c3)c3c2oc2ccccc23)cc1 ; c1ccc(-c2ccc(N(c3ccc4oc5ccccc5c4c3)c3ccc4sc5cccc(-c6ccccc6)c5c4c3)c3c2oc2ccccc23)cc1]

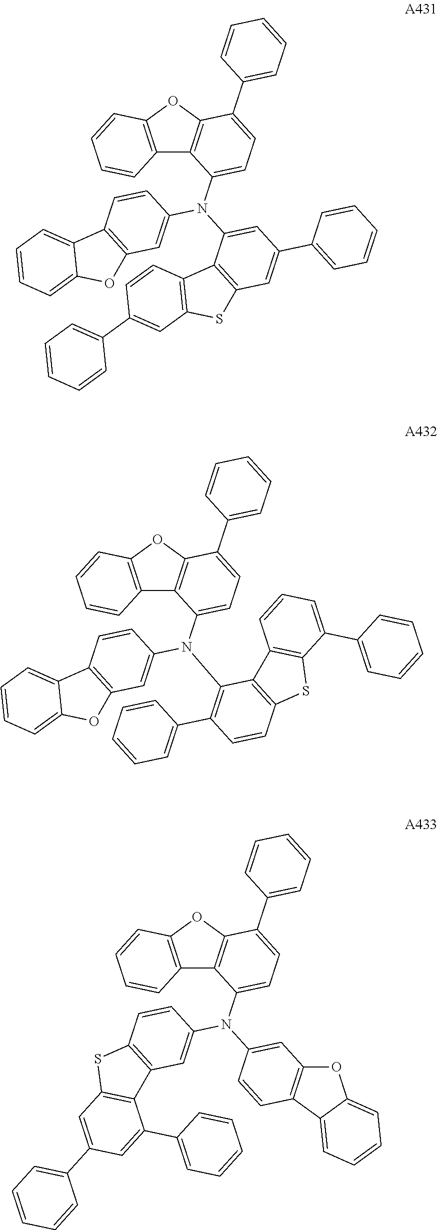 [SMILES: c1ccc(-c2ccc3c(c2)sc2cc(-c4ccccc4)cc(N(c4ccc5c(c4)oc4ccccc45)c4ccc(-c5ccccc5)c5oc6ccccc6c45)c23)cc1 ; c1ccc(-c2ccc3sc4c(-c5ccccc5)cccc4c3c2N(c2ccc3c(c2)oc2ccccc23)c2ccc(-c3ccccc3)c3oc4ccccc4c23)cc1 ; c1ccc(-c2cc(-c3ccccc3)c3c(c2)sc2ccc(N(c4ccc5c(c4)oc4ccccc45)c4ccc(-c5ccccc5)c5oc6ccccc6c45)cc23)cc1]

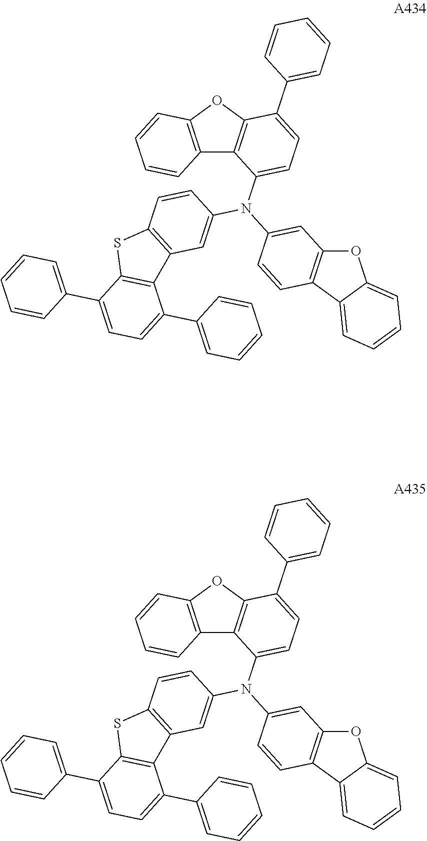 [SMILES: c1ccc(-c2ccc(N(c3ccc4c(c3)oc3ccccc34)c3ccc4sc5c(-c6ccccc6)ccc(-c6ccccc6)c5c4c3)c3c2oc2ccccc23)cc1 ; c1ccc(-c2ccc(N(c3ccc4c(c3)oc3ccccc34)c3ccc4sc5c(-c6ccccc6)ccc(-c6ccccc6)c5c4c3)c3c2oc2ccccc23)cc1]

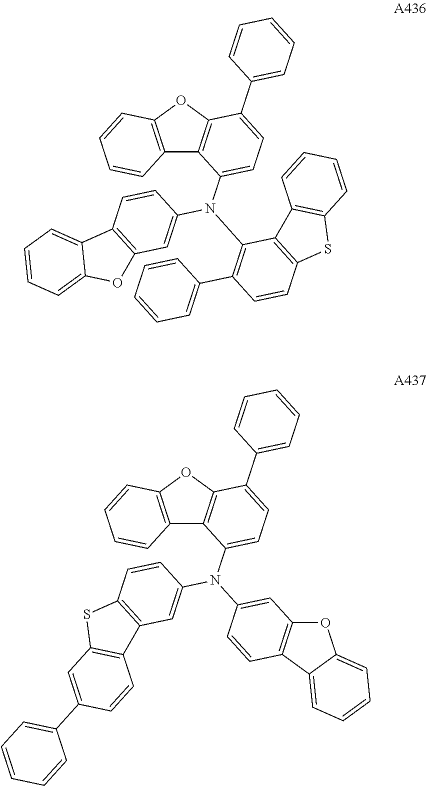 [SMILES: c1ccc(-c2ccc3sc4ccccc4c3c2N(c2ccc3c(c2)oc2ccccc23)c2ccc(-c3ccccc3)c3oc4ccccc4c23)cc1 ; c1ccc(-c2ccc3c(c2)sc2ccc(N(c4ccc5c(c4)oc4ccccc45)c4ccc(-c5ccccc5)c5oc6ccccc6c45)cc23)cc1]

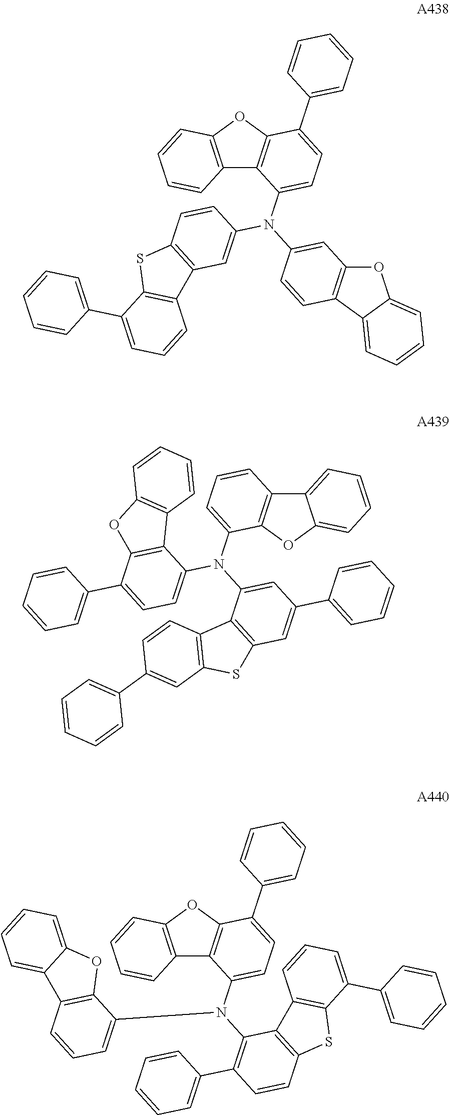 [SMILES: c1ccc(-c2ccc(N(c3ccc4c(c3)oc3ccccc34)c3ccc4sc5c(-c6ccccc6)cccc5c4c3)c3c2oc2ccccc23)cc1 ; c1ccc(-c2ccc3c(c2)sc2cc(-c4ccccc4)cc(N(c4cccc5c4oc4ccccc45)c4ccc(-c5ccccc5)c5oc6ccccc6c45)c23)cc1 ; c1ccc(-c2ccc3sc4c(-c5ccccc5)cccc4c3c2N(c2cccc3c2oc2ccccc23)c2ccc(-c3ccccc3)c3oc4ccccc4c23)cc1]

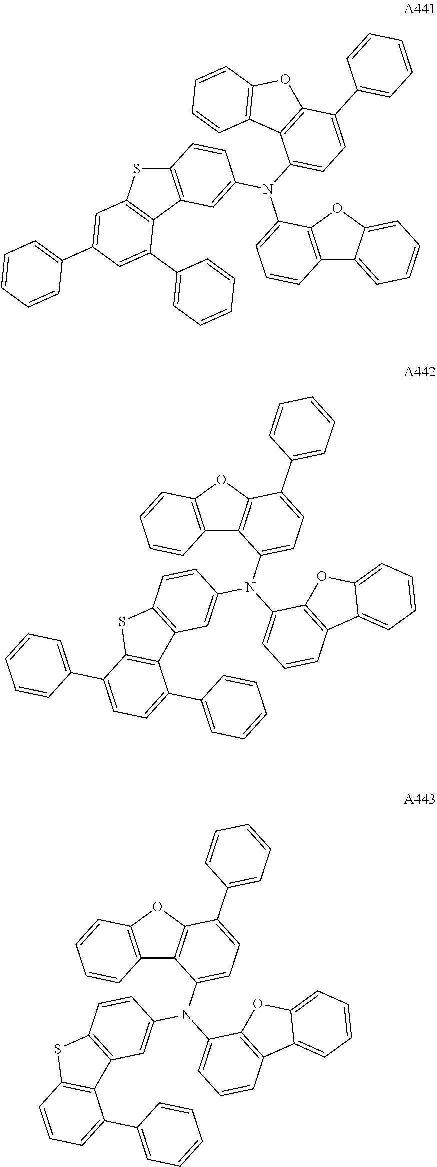 [SMILES: c1ccc(-c2cc(-c3ccccc3)c3c(c2)sc2ccc(N(c4cccc5c4oc4ccccc45)c4ccc(-c5ccccc5)c5oc6ccccc6c45)cc23)cc1 ; c1ccc(-c2ccc(N(c3ccc4sc5c(-c6ccccc6)ccc(-c6ccccc6)c5c4c3)c3cccc4c3oc3ccccc34)c3c2oc2ccccc23)cc1 ; c1ccc(-c2ccc(N(c3ccc4sc5cccc(-c6ccccc6)c5c4c3)c3cccc4c3oc3ccccc34)c3c2oc2ccccc23)cc1]

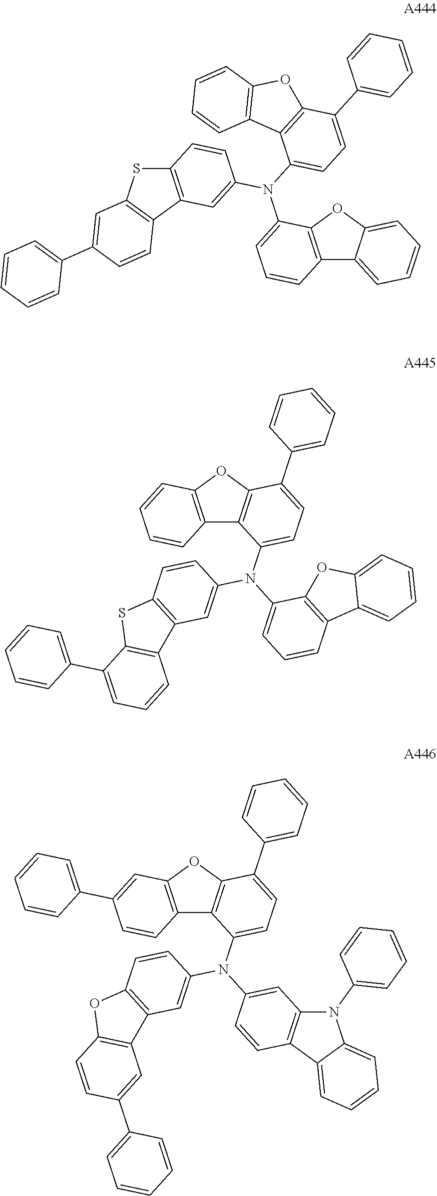 [SMILES: c1ccc(-c2ccc3c(c2)sc2ccc(N(c4cccc5c4oc4ccccc45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4c3oc3ccccc34)c3c2oc2ccccc23)cc1 ; c1ccc(-c2ccc3c(c2)oc2c(-c4ccccc4)ccc(N(c4ccc5oc6ccc(-c7ccccc7)cc6c5c4)c4ccc5c6ccccc6n(-c6ccccc6)c5c4)c23)cc1]

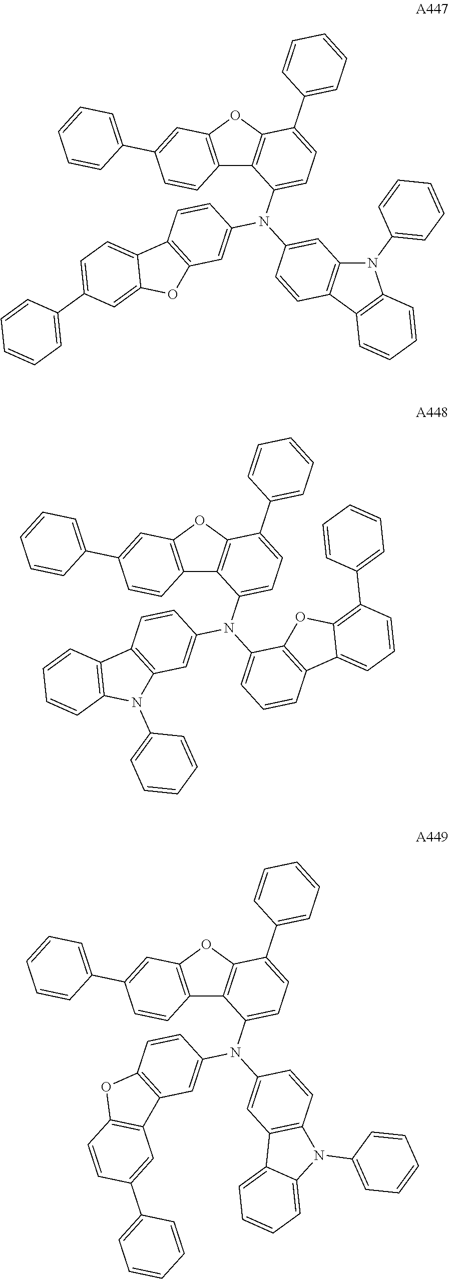 [SMILES: c1ccc(-c2ccc3c(c2)oc2cc(N(c4ccc5c6ccccc6n(-c6ccccc6)c5c4)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4ccc5c6ccccc6n(-c6ccccc6)c5c4)c4cccc5c4oc4c(-c6ccccc6)cccc45)c23)cc1 ; c1ccc(-c2ccc3c(c2)oc2c(-c4ccccc4)ccc(N(c4ccc5oc6ccc(-c7ccccc7)cc6c5c4)c4ccc5c(c4)c4ccccc4n5-c4ccccc4)c23)cc1]

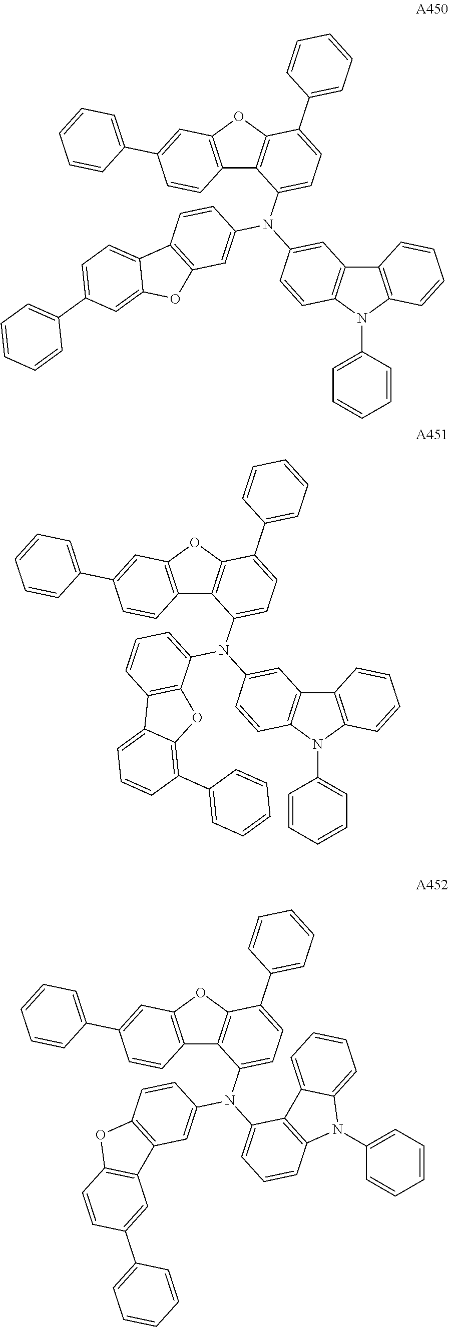 [SMILES: c1ccc(-c2ccc3c(c2)oc2cc(N(c4ccc5c(c4)c4ccccc4n5-c4ccccc4)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4ccc5c(c4)c4ccccc4n5-c4ccccc4)c4cccc5c4oc4c(-c6ccccc6)cccc45)c23)cc1 ; c1ccc(-c2ccc3c(c2)oc2c(-c4ccccc4)ccc(N(c4ccc5oc6ccc(-c7ccccc7)cc6c5c4)c4cccc5c4c4ccccc4n5-c4ccccc4)c23)cc1]

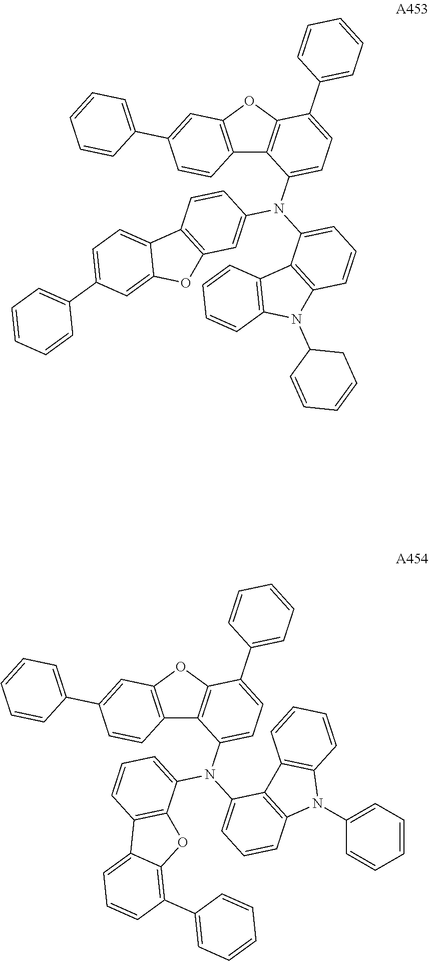 [SMILES: C1=CCC(n2c3ccccc3c3c(N(c4ccc5c(c4)oc4cc(-c6ccccc6)ccc45)c4ccc(-c5ccccc5)c5oc6cc(-c7ccccc7)ccc6c45)cccc32)C=C1 ; c1ccc(-c2ccc3c(c2)oc2c(-c4ccccc4)ccc(N(c4cccc5c4oc4c(-c6ccccc6)cccc45)c4cccc5c4c4ccccc4n5-c4ccccc4)c23)cc1]

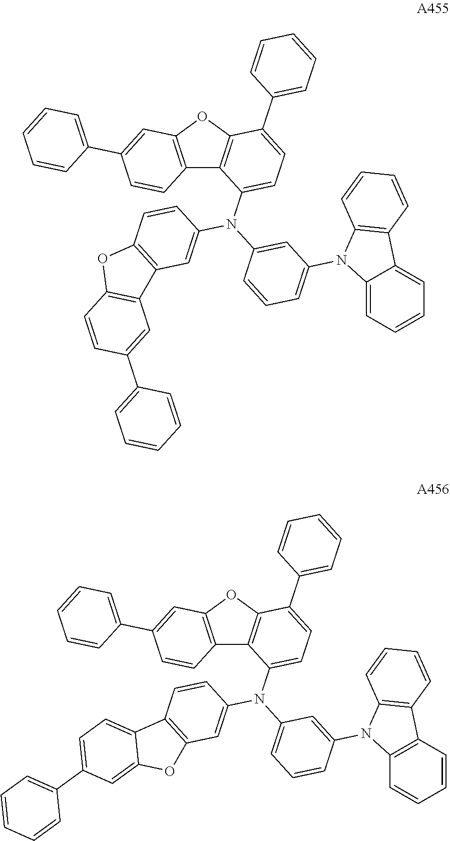 [SMILES: c1ccc(-c2ccc3c(c2)oc2c(-c4ccccc4)ccc(N(c4cccc(-n5c6ccccc6c6ccccc65)c4)c4ccc5oc6ccc(-c7ccccc7)cc6c5c4)c23)cc1 ; c1ccc(-c2ccc3c(c2)oc2cc(N(c4cccc(-n5c6ccccc6c6ccccc65)c4)c4ccc(-c5ccccc5)c5oc6cc(-c7ccccc7)ccc6c45)ccc23)cc1]

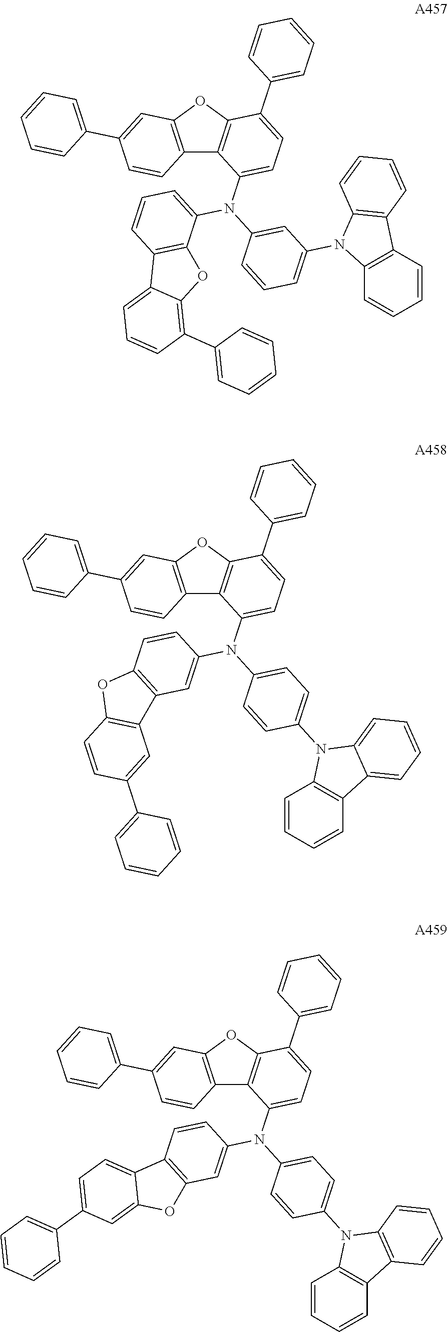 [SMILES: c1ccc(-c2ccc3c(c2)oc2c(-c4ccccc4)ccc(N(c4cccc(-n5c6ccccc6c6ccccc65)c4)c4cccc5c4oc4c(-c6ccccc6)cccc45)c23)cc1 ; c1ccc(-c2ccc3c(c2)oc2c(-c4ccccc4)ccc(N(c4ccc(-n5c6ccccc6c6ccccc65)cc4)c4ccc5oc6ccc(-c7ccccc7)cc6c5c4)c23)cc1 ; c1ccc(-c2ccc3c(c2)oc2cc(N(c4ccc(-n5c6ccccc6c6ccccc65)cc4)c4ccc(-c5ccccc5)c5oc6cc(-c7ccccc7)ccc6c45)ccc23)cc1]

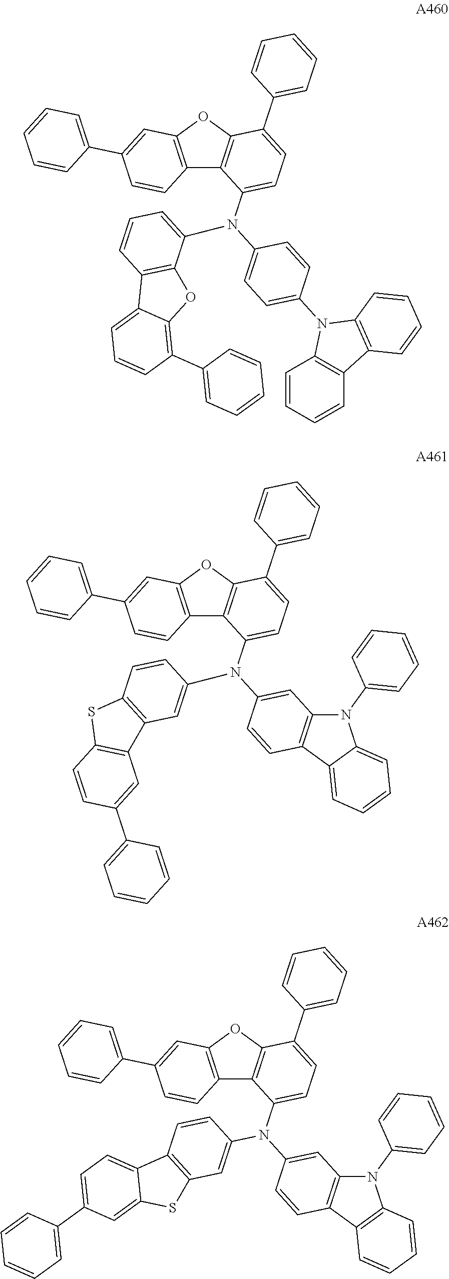 [SMILES: c1ccc(-c2ccc3c(c2)oc2c(-c4ccccc4)ccc(N(c4ccc(-n5c6ccccc6c6ccccc65)cc4)c4cccc5c4oc4c(-c6ccccc6)cccc45)c23)cc1 ; c1ccc(-c2ccc3c(c2)oc2c(-c4ccccc4)ccc(N(c4ccc5sc6ccc(-c7ccccc7)cc6c5c4)c4ccc5c6ccccc6n(-c6ccccc6)c5c4)c23)cc1 ; c1ccc(-c2ccc3c(c2)oc2c(-c4ccccc4)ccc(N(c4ccc5c(c4)sc4cc(-c6ccccc6)ccc45)c4ccc5c6ccccc6n(-c6ccccc6)c5c4)c23)cc1]

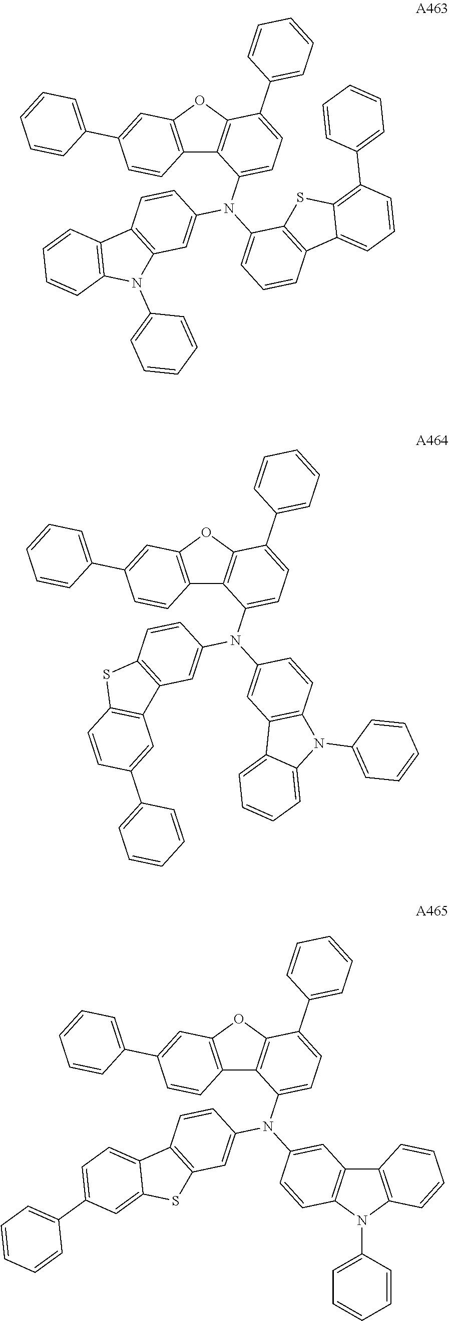 [SMILES: c1ccc(-c2ccc3c(c2)oc2c(-c4ccccc4)ccc(N(c4ccc5c6ccccc6n(-c6ccccc6)c5c4)c4cccc5c4sc4c(-c6ccccc6)cccc45)c23)cc1 ; c1ccc(-c2ccc3c(c2)oc2c(-c4ccccc4)ccc(N(c4ccc5sc6ccc(-c7ccccc7)cc6c5c4)c4ccc5c(c4)c4ccccc4n5-c4ccccc4)c23)cc1 ; c1ccc(-c2ccc3c(c2)oc2c(-c4ccccc4)ccc(N(c4ccc5c(c4)sc4cc(-c6ccccc6)ccc45)c4ccc5c(c4)c4ccccc4n5-c4ccccc4)c23)cc1]

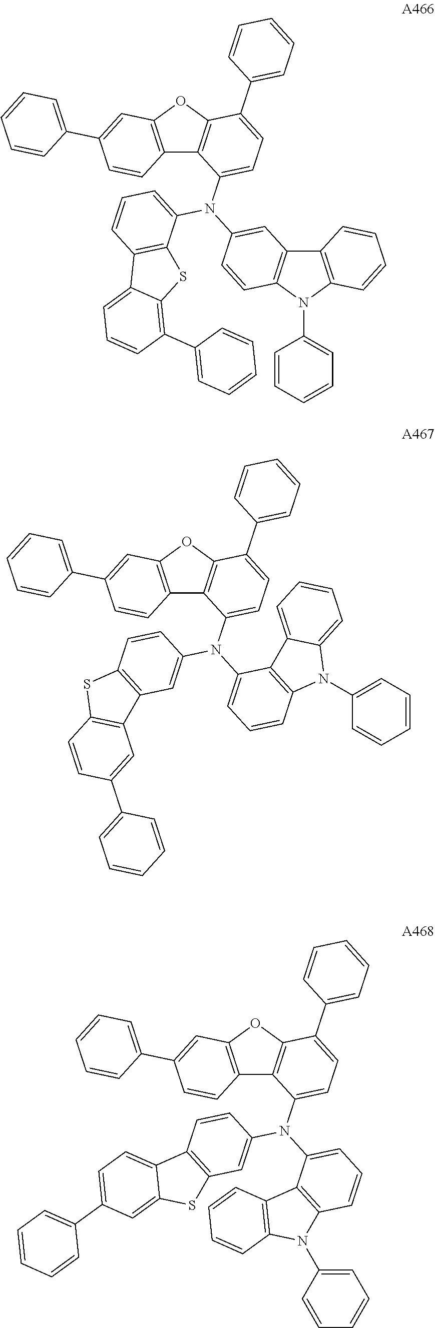 [SMILES: c1ccc(-c2ccc3c(c2)oc2c(-c4ccccc4)ccc(N(c4ccc5c(c4)c4ccccc4n5-c4ccccc4)c4cccc5c4sc4c(-c6ccccc6)cccc45)c23)cc1 ; c1ccc(-c2ccc3c(c2)oc2c(-c4ccccc4)ccc(N(c4ccc5sc6ccc(-c7ccccc7)cc6c5c4)c4cccc5c4c4ccccc4n5-c4ccccc4)c23)cc1 ; c1ccc(-c2ccc3c(c2)oc2c(-c4ccccc4)ccc(N(c4ccc5c(c4)sc4cc(-c6ccccc6)ccc45)c4cccc5c4c4ccccc4n5-c4ccccc4)c23)cc1]

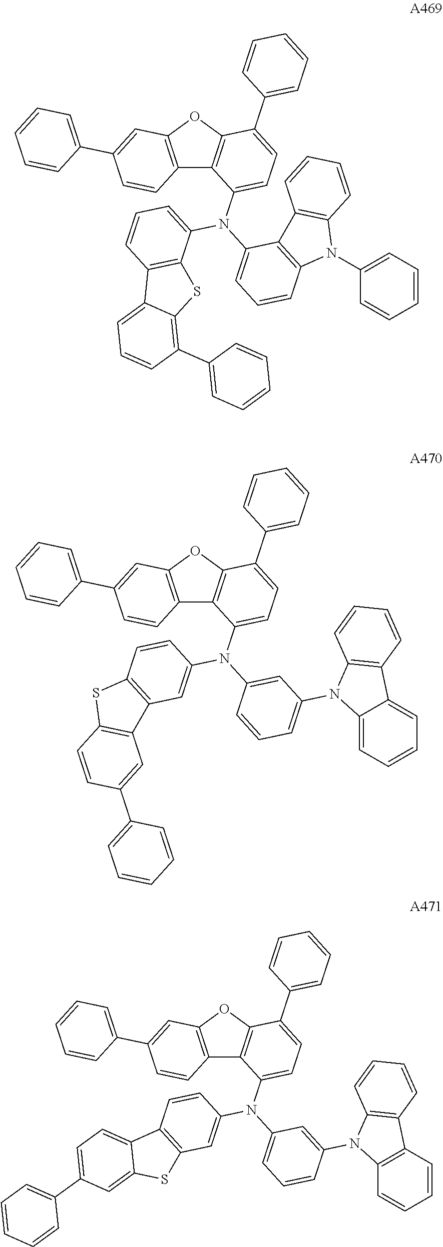 [SMILES: c1ccc(-c2ccc3c(c2)oc2c(-c4ccccc4)ccc(N(c4cccc5c4sc4c(-c6ccccc6)cccc45)c4cccc5c4c4ccccc4n5-c4ccccc4)c23)cc1 ; c1ccc(-c2ccc3c(c2)oc2c(-c4ccccc4)ccc(N(c4cccc(-n5c6ccccc6c6ccccc65)c4)c4ccc5sc6ccc(-c7ccccc7)cc6c5c4)c23)cc1 ; c1ccc(-c2ccc3c(c2)oc2c(-c4ccccc4)ccc(N(c4cccc(-n5c6ccccc6c6ccccc65)c4)c4ccc5c(c4)sc4cc(-c6ccccc6)ccc45)c23)cc1]

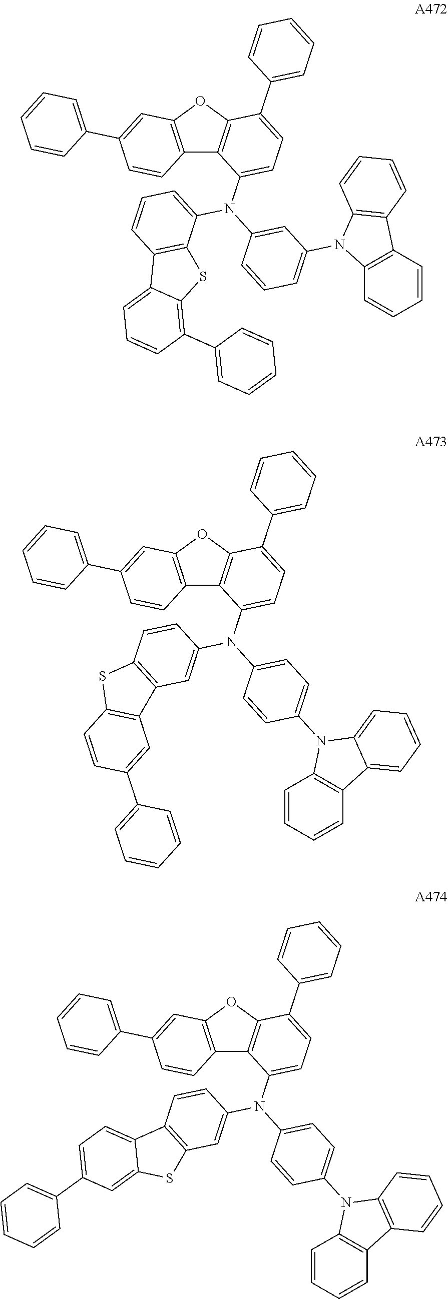 [SMILES: c1ccc(-c2ccc3c(c2)oc2c(-c4ccccc4)ccc(N(c4cccc(-n5c6ccccc6c6ccccc65)c4)c4cccc5c4sc4c(-c6ccccc6)cccc45)c23)cc1 ; c1ccc(-c2ccc3c(c2)oc2c(-c4ccccc4)ccc(N(c4ccc(-n5c6ccccc6c6ccccc65)cc4)c4ccc5sc6ccc(-c7ccccc7)cc6c5c4)c23)cc1 ; c1ccc(-c2ccc3c(c2)oc2c(-c4ccccc4)ccc(N(c4ccc(-n5c6ccccc6c6ccccc65)cc4)c4ccc5c(c4)sc4cc(-c6ccccc6)ccc45)c23)cc1]

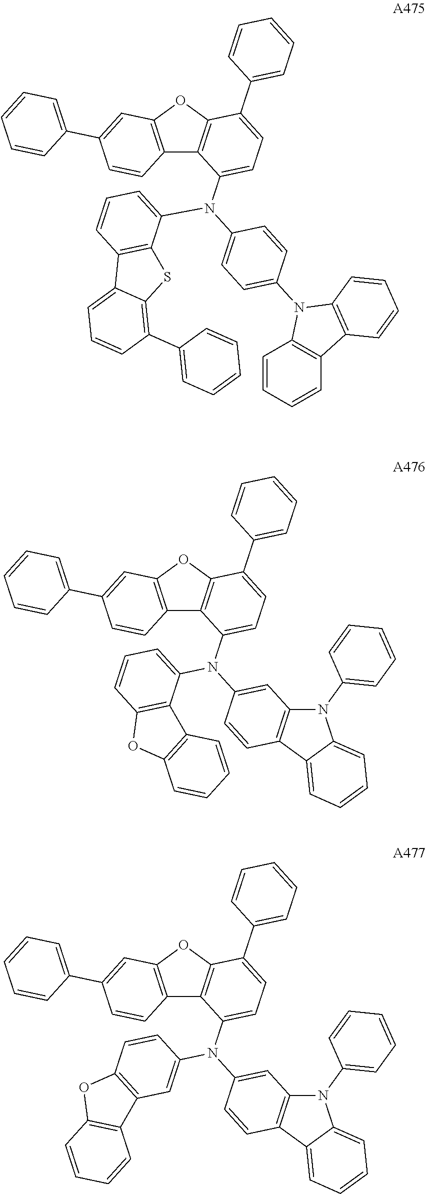 [SMILES: c1ccc(-c2ccc3c(c2)oc2c(-c4ccccc4)ccc(N(c4ccc(-n5c6ccccc6c6ccccc65)cc4)c4cccc5c4sc4c(-c6ccccc6)cccc45)c23)cc1 ; c1ccc(-c2ccc3c(c2)oc2c(-c4ccccc4)ccc(N(c4ccc5c6ccccc6n(-c6ccccc6)c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4ccc5c6ccccc6n(-c6ccccc6)c5c4)c23)cc1]

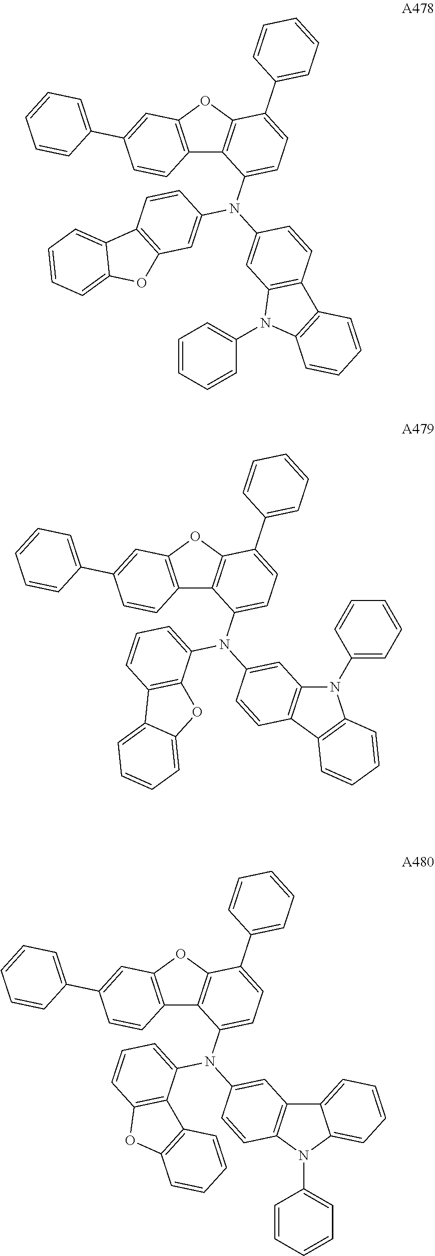 [SMILES: c1ccc(-c2ccc3c(c2)oc2c(-c4ccccc4)ccc(N(c4ccc5c(c4)oc4ccccc45)c4ccc5c6ccccc6n(-c6ccccc6)c5c4)c23)cc1 ; c1ccc(-c2ccc3c(c2)oc2c(-c4ccccc4)ccc(N(c4ccc5c6ccccc6n(-c6ccccc6)c5c4)c4cccc5c4oc4ccccc45)c23)cc1 ; c1ccc(-c2ccc3c(c2)oc2c(-c4ccccc4)ccc(N(c4ccc5c(c4)c4ccccc4n5-c4ccccc4)c4cccc5oc6ccccc6c45)c23)cc1]

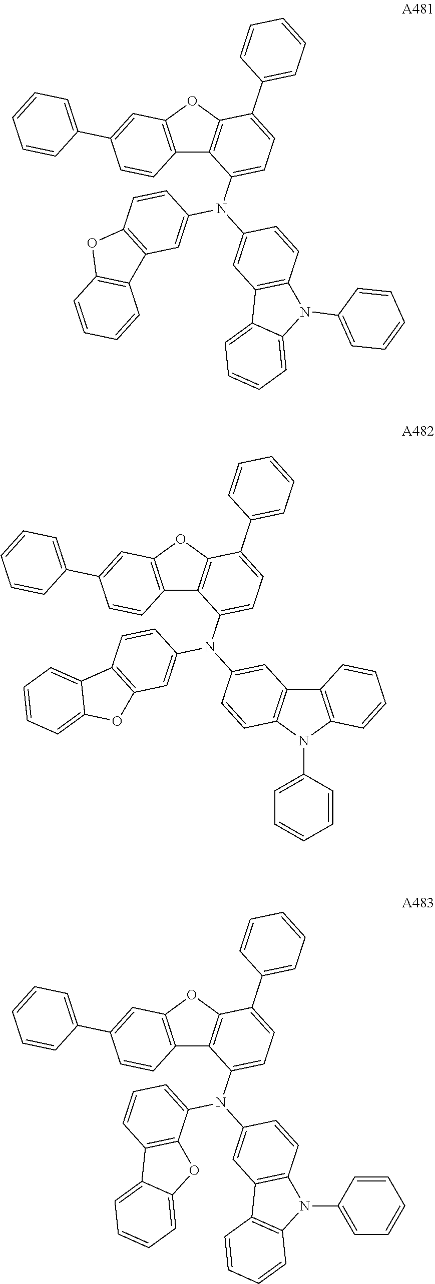 [SMILES: c1ccc(-c2ccc3c(c2)oc2c(-c4ccccc4)ccc(N(c4ccc5oc6ccccc6c5c4)c4ccc5c(c4)c4ccccc4n5-c4ccccc4)c23)cc1 ; c1ccc(-c2ccc3c(c2)oc2c(-c4ccccc4)ccc(N(c4ccc5c(c4)oc4ccccc45)c4ccc5c(c4)c4ccccc4n5-c4ccccc4)c23)cc1 ; c1ccc(-c2ccc3c(c2)oc2c(-c4ccccc4)ccc(N(c4ccc5c(c4)c4ccccc4n5-c4ccccc4)c4cccc5c4oc4ccccc45)c23)cc1]

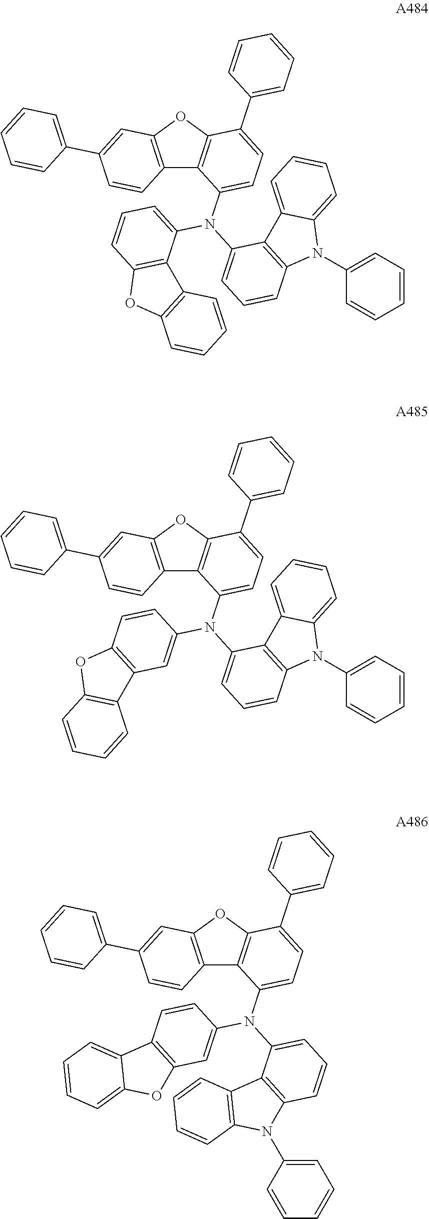 [SMILES: c1ccc(-c2ccc3c(c2)oc2c(-c4ccccc4)ccc(N(c4cccc5oc6ccccc6c45)c4cccc5c4c4ccccc4n5-c4ccccc4)c23)cc1 ; c1ccc(-c2ccc3c(c2)oc2c(-c4ccccc4)ccc(N(c4ccc5oc6ccccc6c5c4)c4cccc5c4c4ccccc4n5-c4ccccc4)c23)cc1 ; c1ccc(-c2ccc3c(c2)oc2c(-c4ccccc4)ccc(N(c4ccc5c(c4)oc4ccccc45)c4cccc5c4c4ccccc4n5-c4ccccc4)c23)cc1]

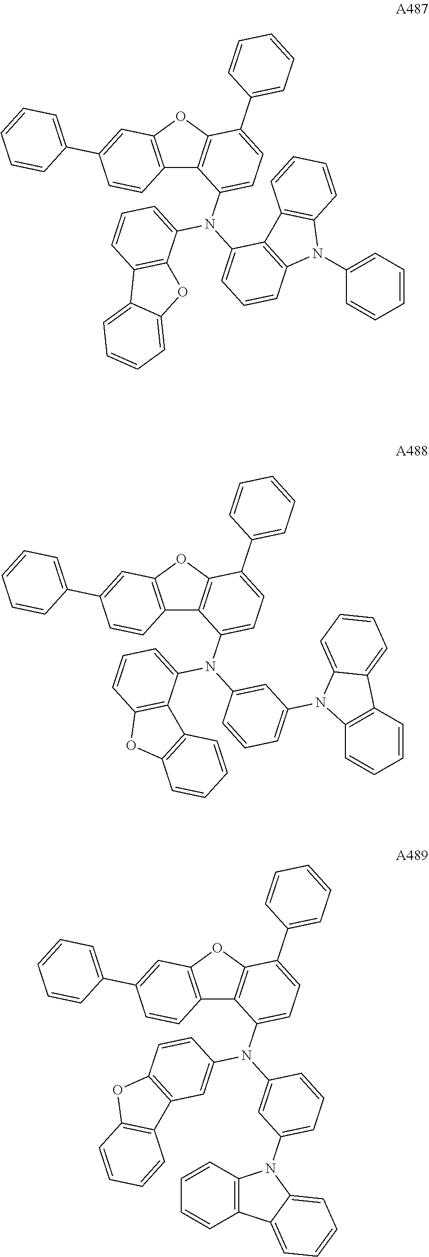 [SMILES: c1ccc(-c2ccc3c(c2)oc2c(-c4ccccc4)ccc(N(c4cccc5c4oc4ccccc45)c4cccc5c4c4ccccc4n5-c4ccccc4)c23)cc1 ; c1ccc(-c2ccc3c(c2)oc2c(-c4ccccc4)ccc(N(c4cccc(-n5c6ccccc6c6ccccc65)c4)c4cccc5oc6ccccc6c45)c23)cc1 ; c1ccc(-c2ccc3c(c2)oc2c(-c4ccccc4)ccc(N(c4cccc(-n5c6ccccc6c6ccccc65)c4)c4ccc5oc6ccccc6c5c4)c23)cc1]

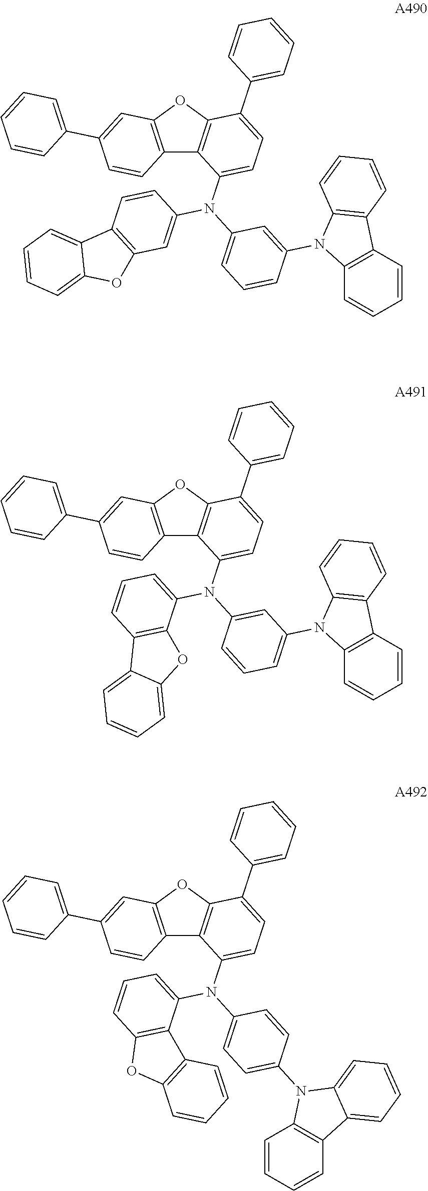 [SMILES: c1ccc(-c2ccc3c(c2)oc2c(-c4ccccc4)ccc(N(c4cccc(-n5c6ccccc6c6ccccc65)c4)c4ccc5c(c4)oc4ccccc45)c23)cc1 ; c1ccc(-c2ccc3c(c2)oc2c(-c4ccccc4)ccc(N(c4cccc(-n5c6ccccc6c6ccccc65)c4)c4cccc5c4oc4ccccc45)c23)cc1 ; c1ccc(-c2ccc3c(c2)oc2c(-c4ccccc4)ccc(N(c4ccc(-n5c6ccccc6c6ccccc65)cc4)c4cccc5oc6ccccc6c45)c23)cc1]

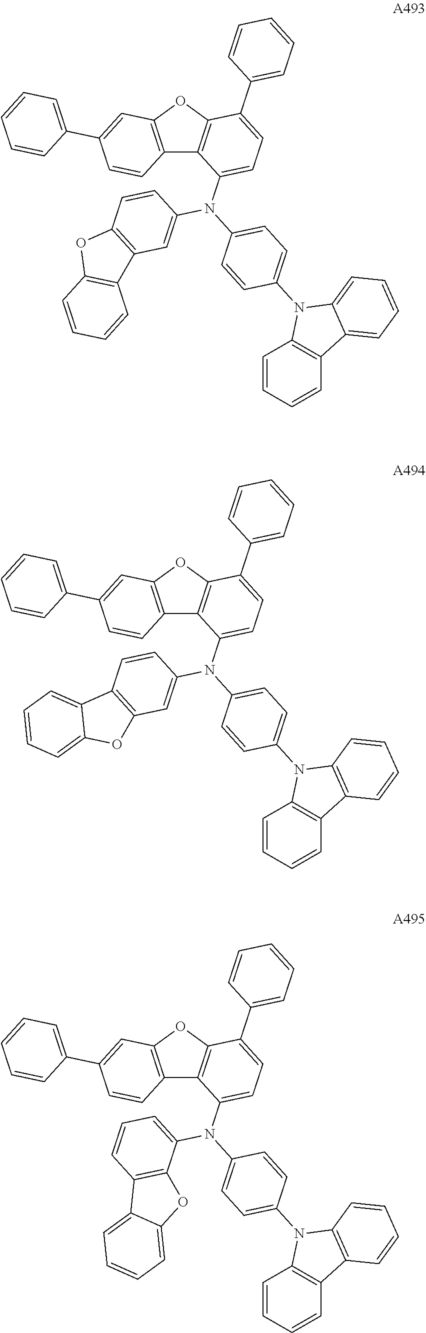 [SMILES: c1ccc(-c2ccc3c(c2)oc2c(-c4ccccc4)ccc(N(c4ccc(-n5c6ccccc6c6ccccc65)cc4)c4ccc5oc6ccccc6c5c4)c23)cc1 ; c1ccc(-c2ccc3c(c2)oc2c(-c4ccccc4)ccc(N(c4ccc(-n5c6ccccc6c6ccccc65)cc4)c4ccc5c(c4)oc4ccccc45)c23)cc1 ; c1ccc(-c2ccc3c(c2)oc2c(-c4ccccc4)ccc(N(c4ccc(-n5c6ccccc6c6ccccc65)cc4)c4cccc5c4oc4ccccc45)c23)cc1]

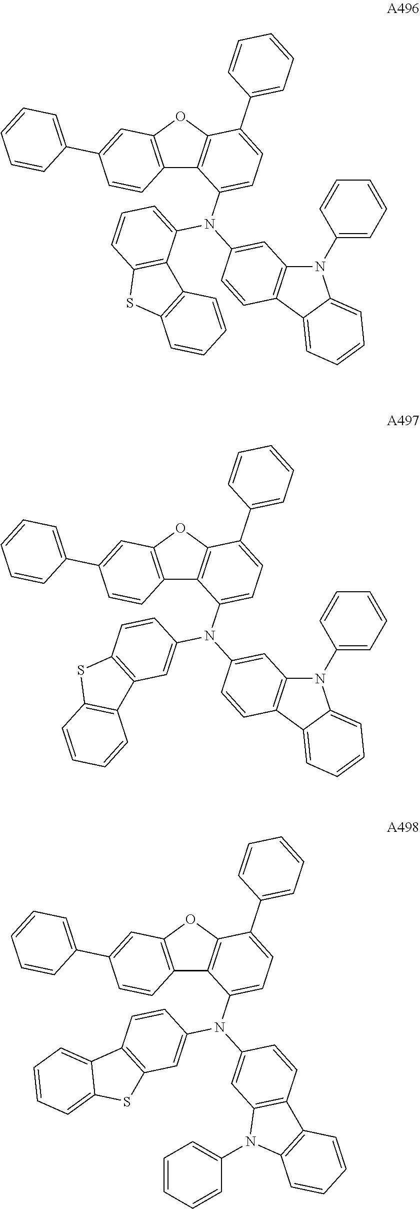 [SMILES: c1ccc(-c2ccc3c(c2)oc2c(-c4ccccc4)ccc(N(c4ccc5c6ccccc6n(-c6ccccc6)c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4ccc5c6ccccc6n(-c6ccccc6)c5c4)c23)cc1 ; c1ccc(-c2ccc3c(c2)oc2c(-c4ccccc4)ccc(N(c4ccc5c(c4)sc4ccccc45)c4ccc5c6ccccc6n(-c6ccccc6)c5c4)c23)cc1]

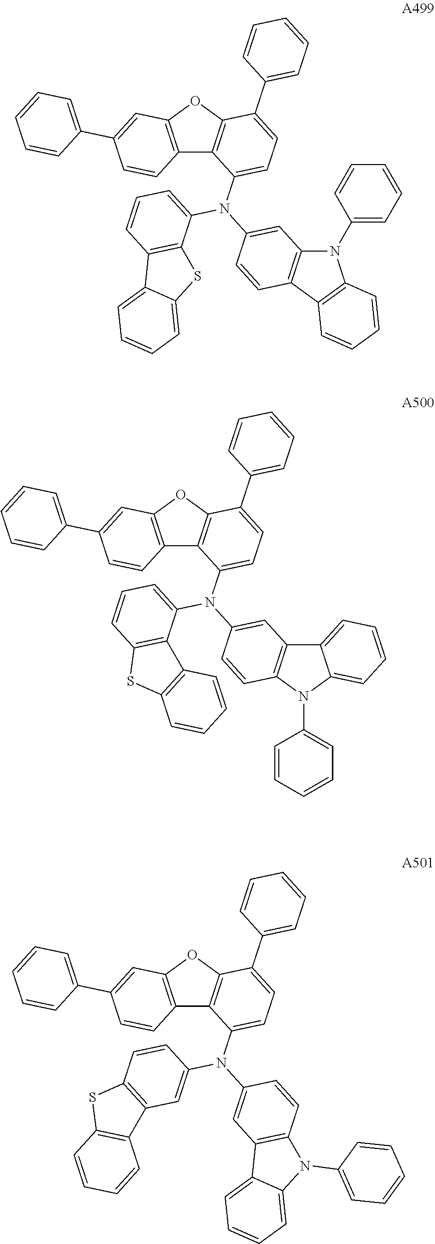 [SMILES: c1ccc(-c2ccc3c(c2)oc2c(-c4ccccc4)ccc(N(c4ccc5c6ccccc6n(-c6ccccc6)c5c4)c4cccc5c4sc4ccccc45)c23)cc1 ; c1ccc(-c2ccc3c(c2)oc2c(-c4ccccc4)ccc(N(c4ccc5c(c4)c4ccccc4n5-c4ccccc4)c4cccc5sc6ccccc6c45)c23)cc1 ; c1ccc(-c2ccc3c(c2)oc2c(-c4ccccc4)ccc(N(c4ccc5sc6ccccc6c5c4)c4ccc5c(c4)c4ccccc4n5-c4ccccc4)c23)cc1]

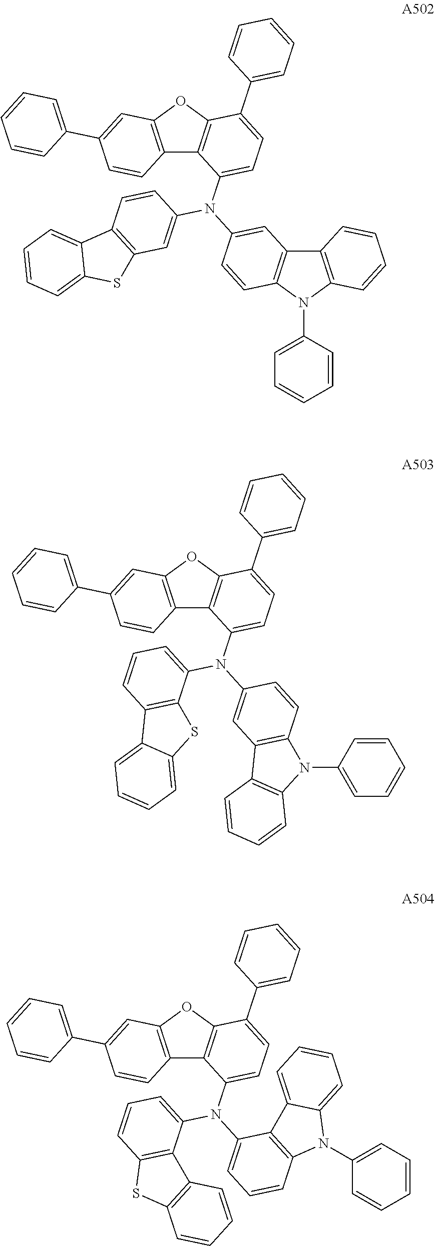 [SMILES: c1ccc(-c2ccc3c(c2)oc2c(-c4ccccc4)ccc(N(c4ccc5c(c4)sc4ccccc45)c4ccc5c(c4)c4ccccc4n5-c4ccccc4)c23)cc1 ; c1ccc(-c2ccc3c(c2)oc2c(-c4ccccc4)ccc(N(c4ccc5c(c4)c4ccccc4n5-c4ccccc4)c4cccc5c4sc4ccccc45)c23)cc1 ; c1ccc(-c2ccc3c(c2)oc2c(-c4ccccc4)ccc(N(c4cccc5sc6ccccc6c45)c4cccc5c4c4ccccc4n5-c4ccccc4)c23)cc1]

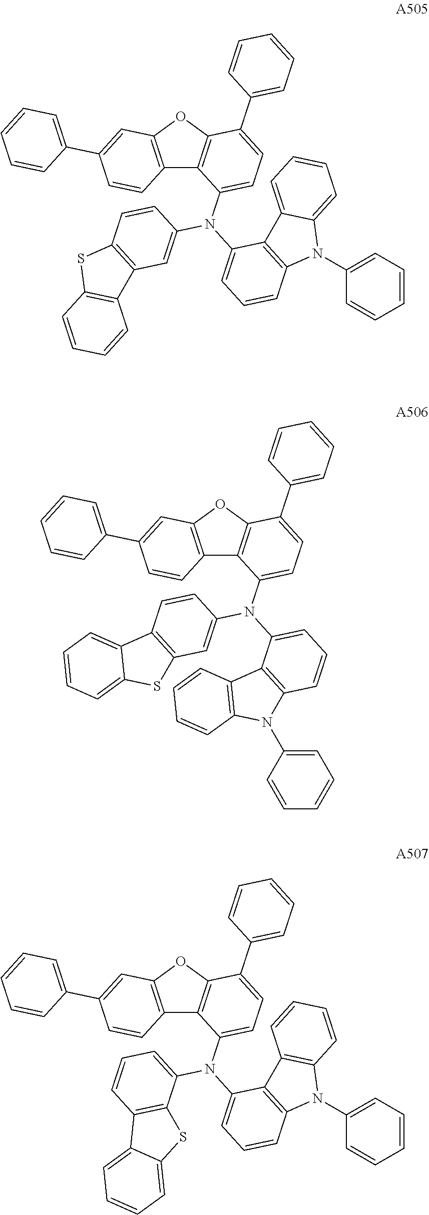 [SMILES: c1ccc(-c2ccc3c(c2)oc2c(-c4ccccc4)ccc(N(c4ccc5sc6ccccc6c5c4)c4cccc5c4c4ccccc4n5-c4ccccc4)c23)cc1 ; c1ccc(-c2ccc3c(c2)oc2c(-c4ccccc4)ccc(N(c4ccc5c(c4)sc4ccccc45)c4cccc5c4c4ccccc4n5-c4ccccc4)c23)cc1 ; c1ccc(-c2ccc3c(c2)oc2c(-c4ccccc4)ccc(N(c4cccc5c4sc4ccccc45)c4cccc5c4c4ccccc4n5-c4ccccc4)c23)cc1]

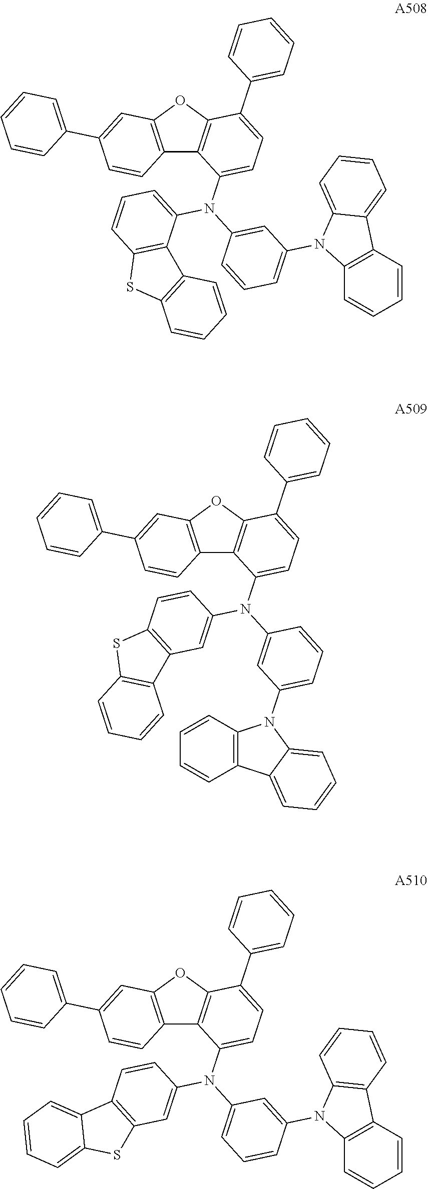 [SMILES: c1ccc(-c2ccc3c(c2)oc2c(-c4ccccc4)ccc(N(c4cccc(-n5c6ccccc6c6ccccc65)c4)c4cccc5sc6ccccc6c45)c23)cc1 ; c1ccc(-c2ccc3c(c2)oc2c(-c4ccccc4)ccc(N(c4cccc(-n5c6ccccc6c6ccccc65)c4)c4ccc5sc6ccccc6c5c4)c23)cc1 ; c1ccc(-c2ccc3c(c2)oc2c(-c4ccccc4)ccc(N(c4cccc(-n5c6ccccc6c6ccccc65)c4)c4ccc5c(c4)sc4ccccc45)c23)cc1]

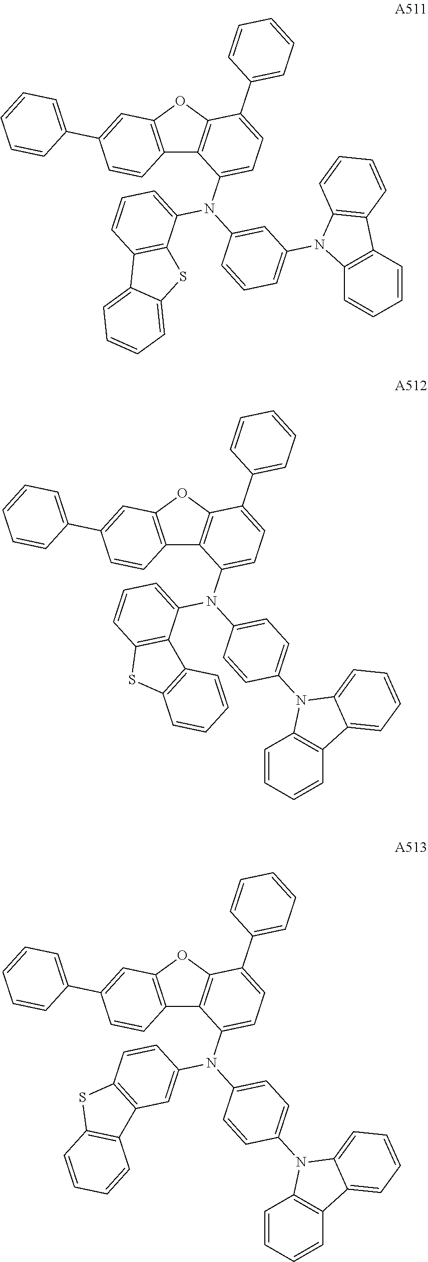 [SMILES: c1ccc(-c2ccc3c(c2)oc2c(-c4ccccc4)ccc(N(c4cccc(-n5c6ccccc6c6ccccc65)c4)c4cccc5c4sc4ccccc45)c23)cc1 ; c1ccc(-c2ccc3c(c2)oc2c(-c4ccccc4)ccc(N(c4ccc(-n5c6ccccc6c6ccccc65)cc4)c4cccc5sc6ccccc6c45)c23)cc1 ; c1ccc(-c2ccc3c(c2)oc2c(-c4ccccc4)ccc(N(c4ccc(-n5c6ccccc6c6ccccc65)cc4)c4ccc5sc6ccccc6c5c4)c23)cc1]

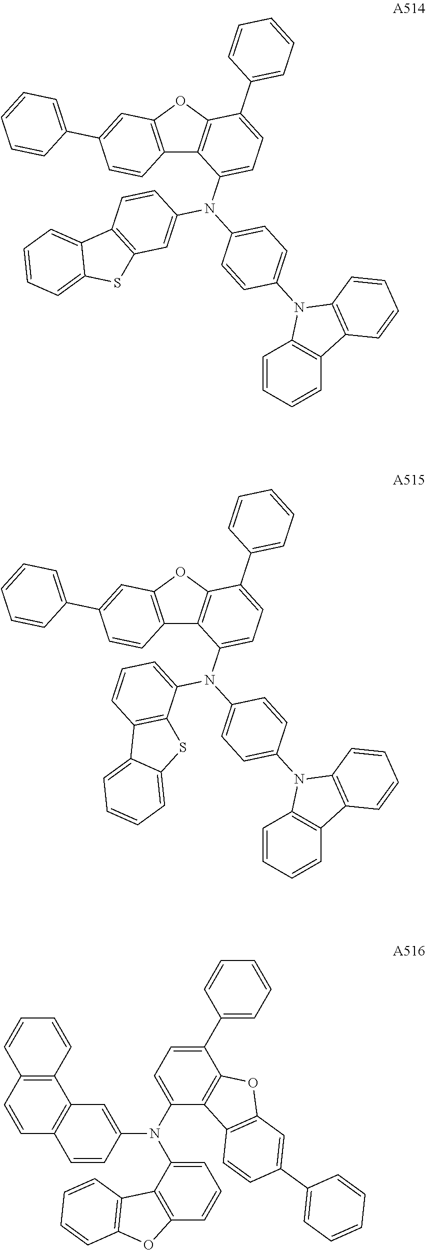 [SMILES: c1ccc(-c2ccc3c(c2)oc2c(-c4ccccc4)ccc(N(c4ccc(-n5c6ccccc6c6ccccc65)cc4)c4ccc5c(c4)sc4ccccc45)c23)cc1 ; c1ccc(-c2ccc3c(c2)oc2c(-c4ccccc4)ccc(N(c4ccc(-n5c6ccccc6c6ccccc65)cc4)c4cccc5c4sc4ccccc45)c23)cc1 ; c1ccc(-c2ccc3c(c2)oc2c(-c4ccccc4)ccc(N(c4ccc5ccc6ccccc6c5c4)c4cccc5oc6ccccc6c45)c23)cc1]

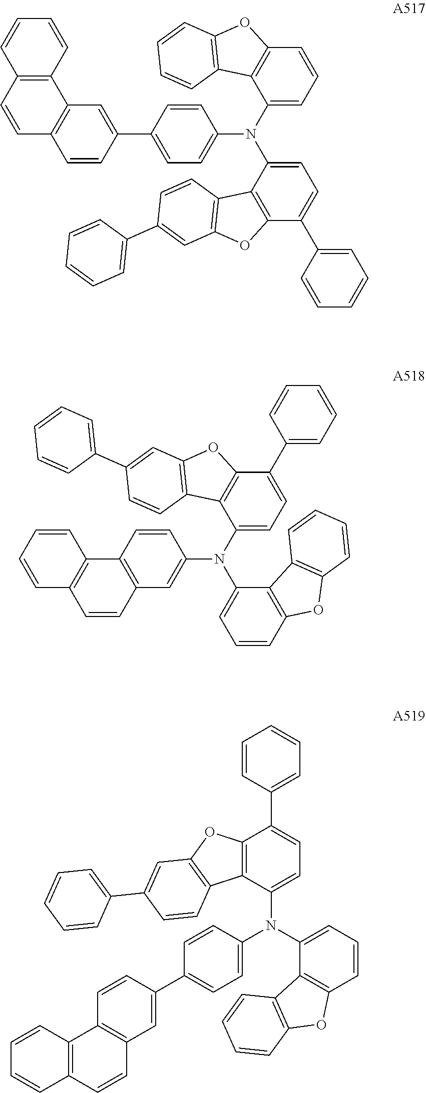 [SMILES: c1ccc(-c2ccc3c(c2)oc2c(-c4ccccc4)ccc(N(c4ccc(-c5ccc6ccc7ccccc7c6c5)cc4)c4cccc5oc6ccccc6c45)c23)cc1 ; c1ccc(-c2ccc3c(c2)oc2c(-c4ccccc4)ccc(N(c4ccc5c(ccc6ccccc65)c4)c4cccc5oc6ccccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5oc6ccccc6c45)c23)cc1]

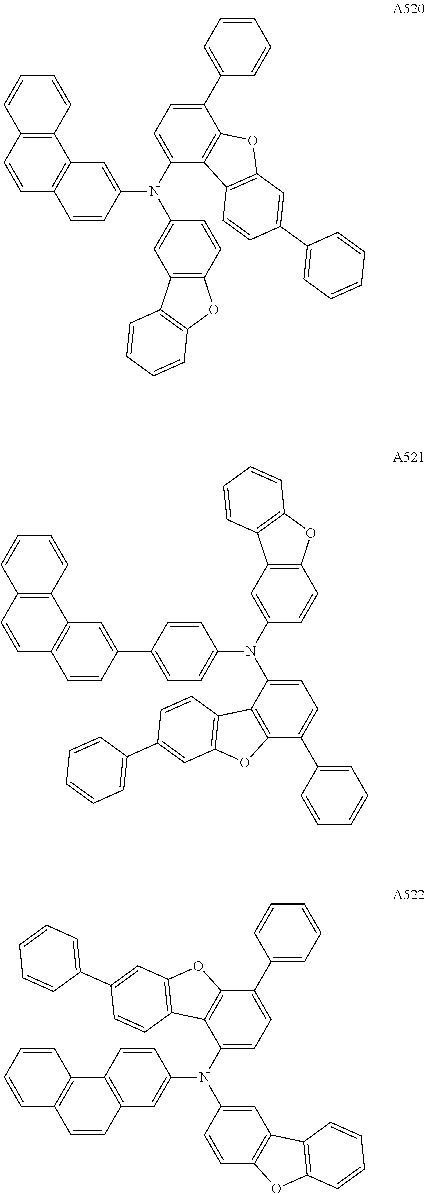 [SMILES: c1ccc(-c2ccc3c(c2)oc2c(-c4ccccc4)ccc(N(c4ccc5ccc6ccccc6c5c4)c4ccc5oc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5oc6ccccc6c5c4)c23)cc1 ; c1ccc(-c2ccc3c(c2)oc2c(-c4ccccc4)ccc(N(c4ccc5c(ccc6ccccc65)c4)c4ccc5oc6ccccc6c5c4)c23)cc1]

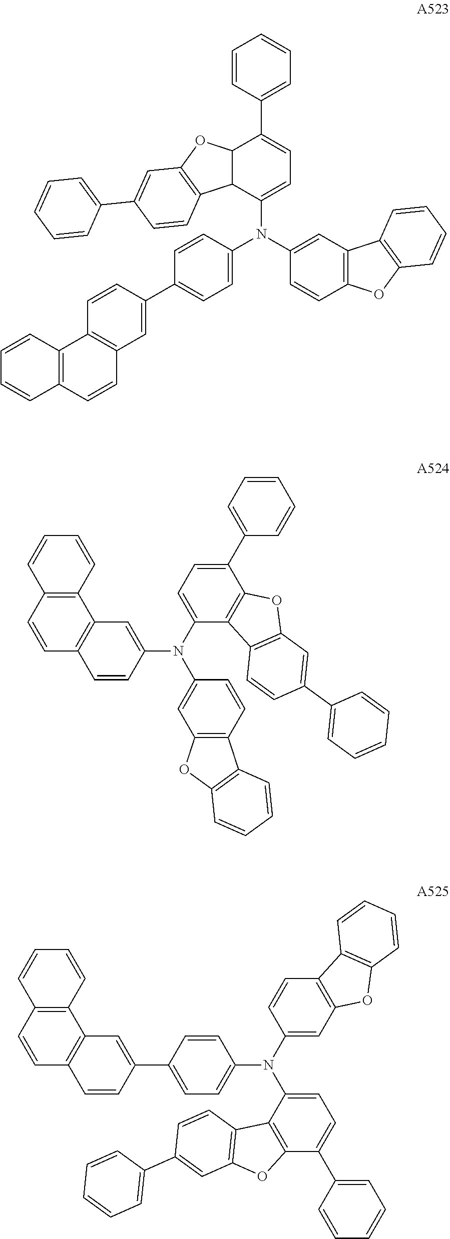 [SMILES: C1=C(c2ccccc2)C2Oc3cc(-c4ccccc4)ccc3C2C(N(c2ccc(-c3ccc4c(ccc5ccccc54)c3)cc2)c2ccc3oc4ccccc4c3c2)=C1 ; c1ccc(-c2ccc3c(c2)oc2c(-c4ccccc4)ccc(N(c4ccc5c(c4)oc4ccccc45)c4ccc5ccc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5c(c4)oc4ccccc45)c23)cc1]

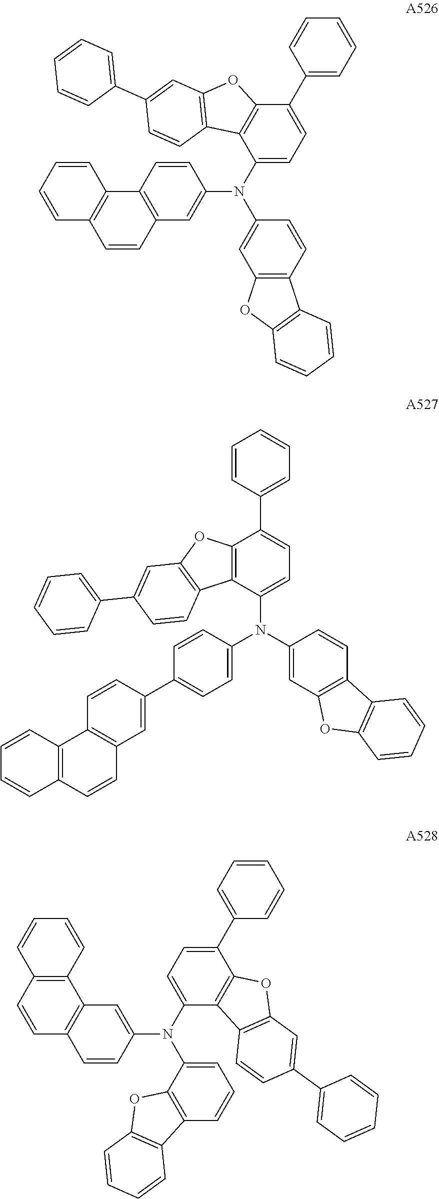 [SMILES: c1ccc(-c2ccc3c(c2)oc2c(-c4ccccc4)ccc(N(c4ccc5c(ccc6ccccc65)c4)c4ccc5c(c4)oc4ccccc45)c23)cc1 ; c1ccc(-c2ccc3c(c2)oc2c(-c4ccccc4)ccc(N(c4ccc(-c5ccc6c(ccc7ccccc76)c5)cc4)c4ccc5c(c4)oc4ccccc45)c23)cc1 ; c1ccc(-c2ccc3c(c2)oc2c(-c4ccccc4)ccc(N(c4ccc5ccc6ccccc6c5c4)c4cccc5c4oc4ccccc45)c23)cc1]

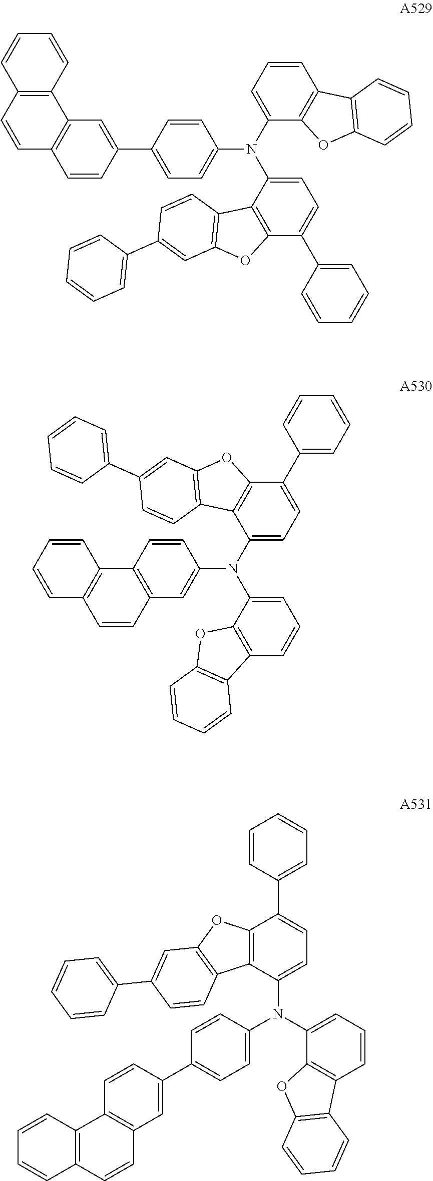 [SMILES: c1ccc(-c2ccc3c(c2)oc2c(-c4ccccc4)ccc(N(c4ccc(-c5ccc6ccc7ccccc7c6c5)cc4)c4cccc5c4oc4ccccc45)c23)cc1 ; c1ccc(-c2ccc3c(c2)oc2c(-c4ccccc4)ccc(N(c4ccc5c(ccc6ccccc65)c4)c4cccc5c4oc4ccccc45)c23)cc1 ; c1ccc(-c2ccc3c(c2)oc2c(-c4ccccc4)ccc(N(c4ccc(-c5ccc6c(ccc7ccccc76)c5)cc4)c4cccc5c4oc4ccccc45)c23)cc1]

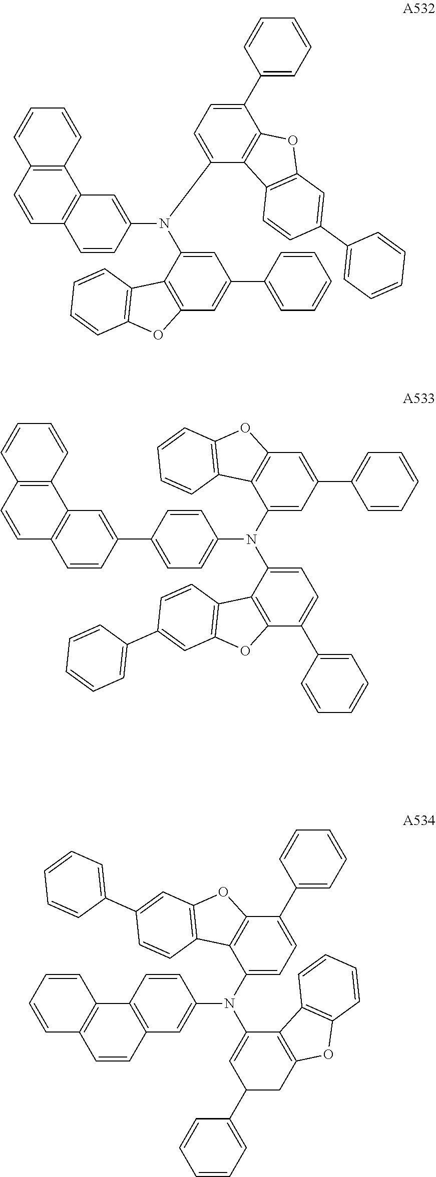 [SMILES: c1ccc(-c2ccc3c(c2)oc2c(-c4ccccc4)ccc(N(c4ccc5ccc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc(-c5ccc6ccc7ccccc7c6c5)cc4)c4cc(-c5ccccc5)cc5oc6ccccc6c45)c23)cc1 ; C1=C(N(c2ccc3c(ccc4ccccc43)c2)c2ccc(-c3ccccc3)c3oc4cc(-c5ccccc5)ccc4c23)c2c(oc3ccccc23)CC1c1ccccc1]

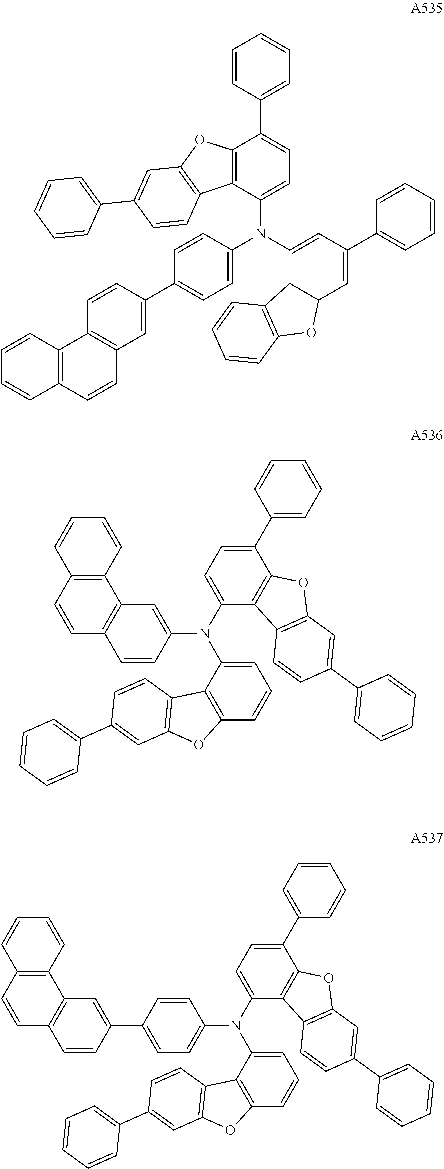 [SMILES: C(=C\N(c1ccc(-c2ccc3c(ccc4ccccc43)c2)cc1)c1ccc(-c2ccccc2)c2oc3cc(-c4ccccc4)ccc3c12)/C(=C\C1Cc2ccccc2O1)c1ccccc1 ; c1ccc(-c2ccc3c(c2)oc2cccc(N(c4ccc5ccc6ccccc6c5c4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5oc6cc(-c7ccccc7)ccc6c45)c23)cc1]

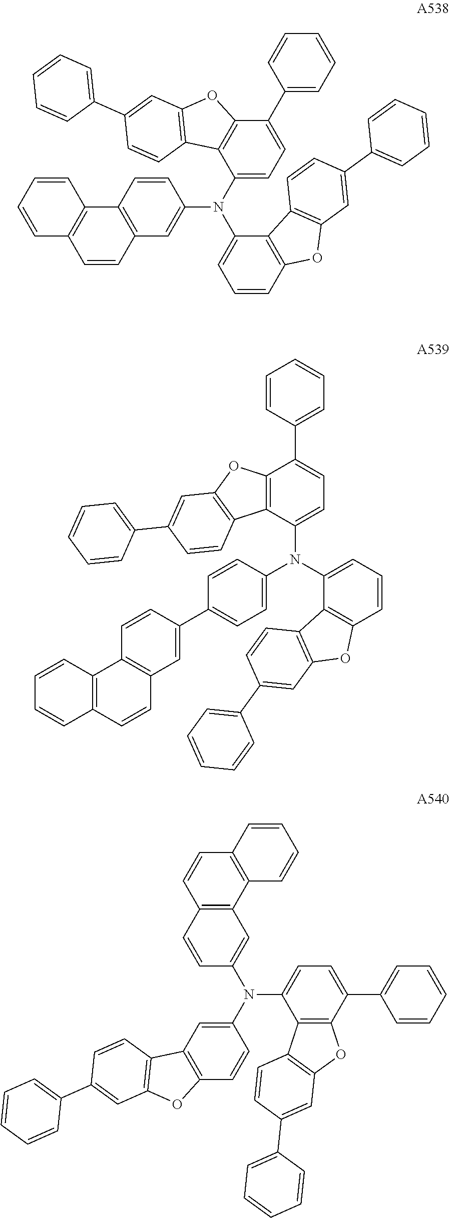 [SMILES: c1ccc(-c2ccc3c(c2)oc2cccc(N(c4ccc5c(ccc6ccccc65)c4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5oc6cc(-c7ccccc7)ccc6c45)c23)cc1 ; c1ccc(-c2ccc3c(c2)oc2ccc(N(c4ccc5ccc6ccccc6c5c4)c4ccc(-c5ccccc5)c5oc6cc(-c7ccccc7)ccc6c45)cc23)cc1]

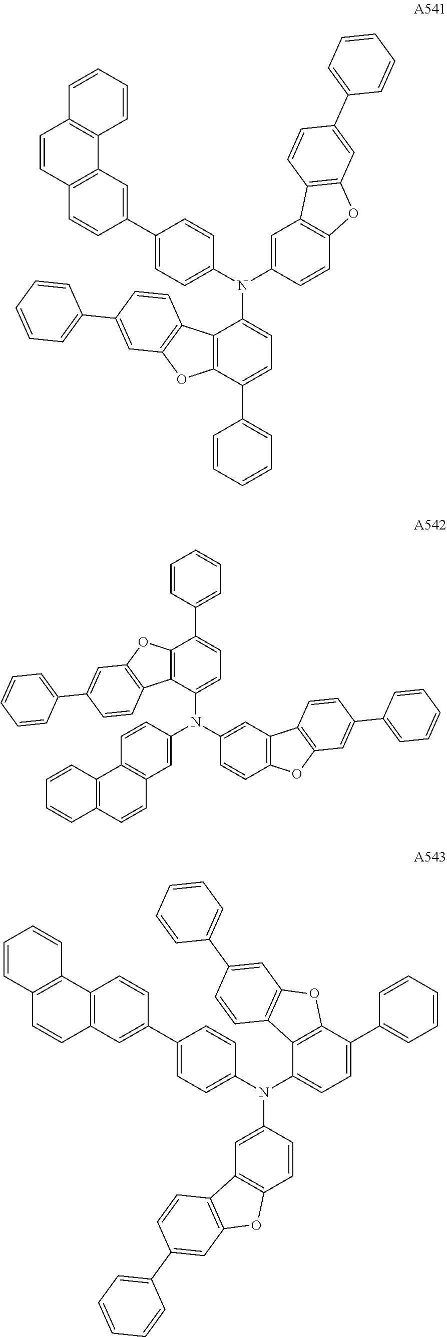 [SMILES: c1ccc(-c2ccc3c(c2)oc2ccc(N(c4ccc(-c5ccc6ccc7ccccc7c6c5)cc4)c4ccc(-c5ccccc5)c5oc6cc(-c7ccccc7)ccc6c45)cc23)cc1 ; c1ccc(-c2ccc3c(c2)oc2ccc(N(c4ccc5c(ccc6ccccc65)c4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5oc6cc(-c7ccccc7)ccc6c45)cc23)cc1]

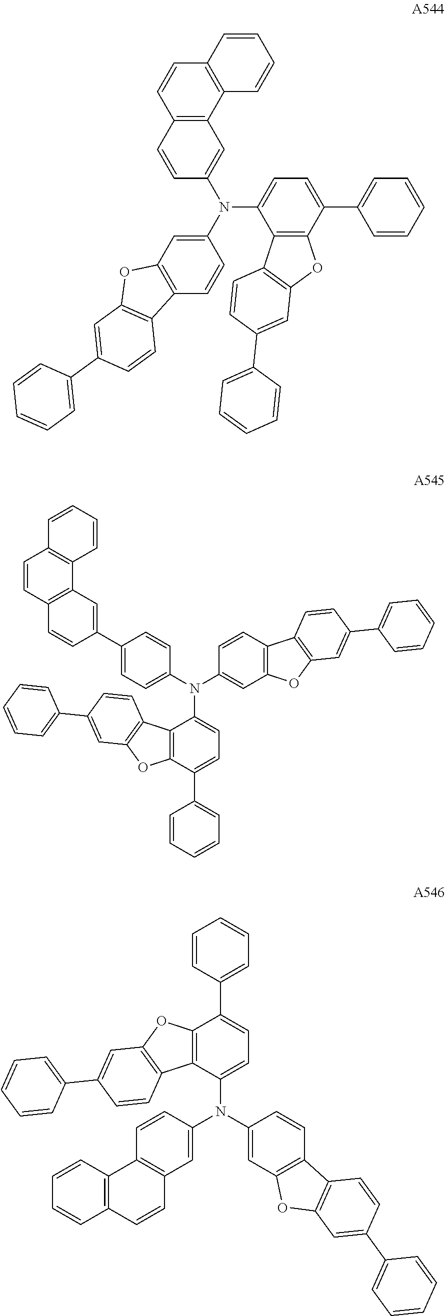 [SMILES: c1ccc(-c2ccc3c(c2)oc2cc(N(c4ccc5ccc6ccccc6c5c4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6ccc7ccccc7c6c5)cc4)c4ccc(-c5ccccc5)c5oc6cc(-c7ccccc7)ccc6c45)ccc23)cc1 ; c1ccc(-c2ccc3c(c2)oc2cc(N(c4ccc5c(ccc6ccccc65)c4)c4ccc(-c5ccccc5)c5oc6cc(-c7ccccc7)ccc6c45)ccc23)cc1]

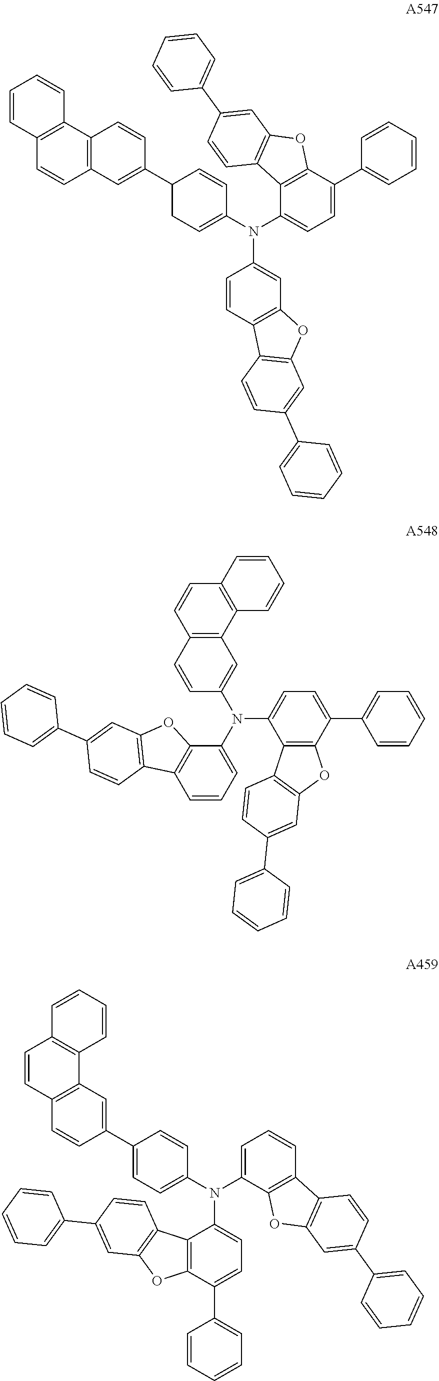 [SMILES: C1=CC(c2ccc3c(ccc4ccccc43)c2)CC=C1N(c1ccc2c(c1)oc1cc(-c3ccccc3)ccc12)c1ccc(-c2ccccc2)c2oc3cc(-c4ccccc4)ccc3c12 ; c1ccc(-c2ccc3c(c2)oc2c(N(c4ccc5ccc6ccccc6c5c4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5oc6cc(-c7ccccc7)ccc6c45)cccc23)cc1]

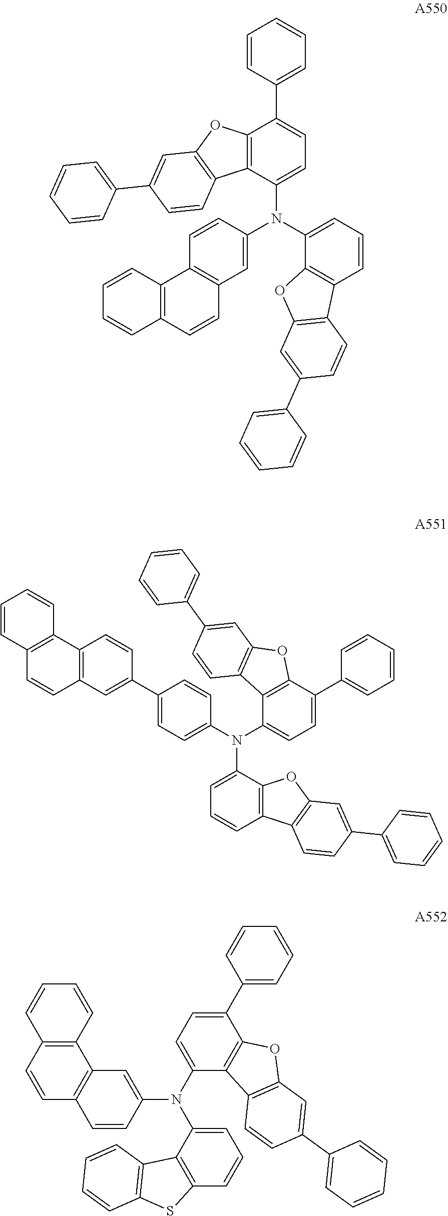 [SMILES: c1ccc(-c2ccc3c(c2)oc2c(N(c4ccc5c(ccc6ccccc65)c4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6c(ccc7ccccc76)c5)cc4)c4ccc(-c5ccccc5)c5oc6cc(-c7ccccc7)ccc6c45)cccc23)cc1 ; c1ccc(-c2ccc3c(c2)oc2c(-c4ccccc4)ccc(N(c4ccc5ccc6ccccc6c5c4)c4cccc5sc6ccccc6c45)c23)cc1]

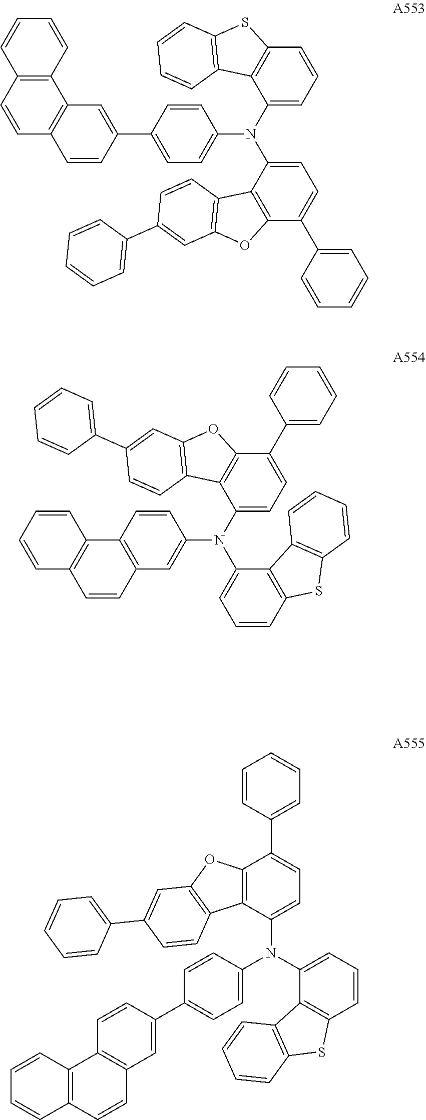 [SMILES: c1ccc(-c2ccc3c(c2)oc2c(-c4ccccc4)ccc(N(c4ccc(-c5ccc6ccc7ccccc7c6c5)cc4)c4cccc5sc6ccccc6c45)c23)cc1 ; c1ccc(-c2ccc3c(c2)oc2c(-c4ccccc4)ccc(N(c4ccc5c(ccc6ccccc65)c4)c4cccc5sc6ccccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5sc6ccccc6c45)c23)cc1]

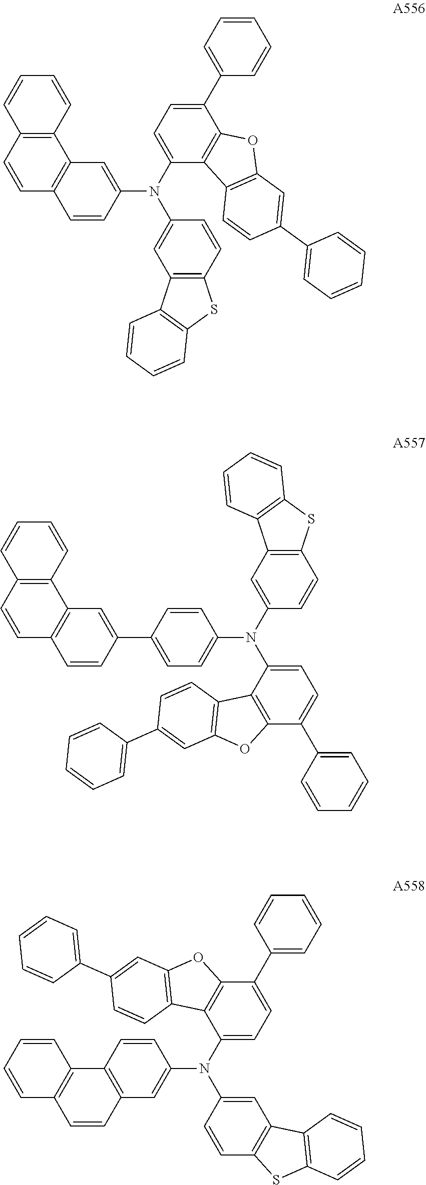 [SMILES: c1ccc(-c2ccc3c(c2)oc2c(-c4ccccc4)ccc(N(c4ccc5ccc6ccccc6c5c4)c4ccc5sc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5sc6ccccc6c5c4)c23)cc1 ; c1ccc(-c2ccc3c(c2)oc2c(-c4ccccc4)ccc(N(c4ccc5c(ccc6ccccc65)c4)c4ccc5sc6ccccc6c5c4)c23)cc1]

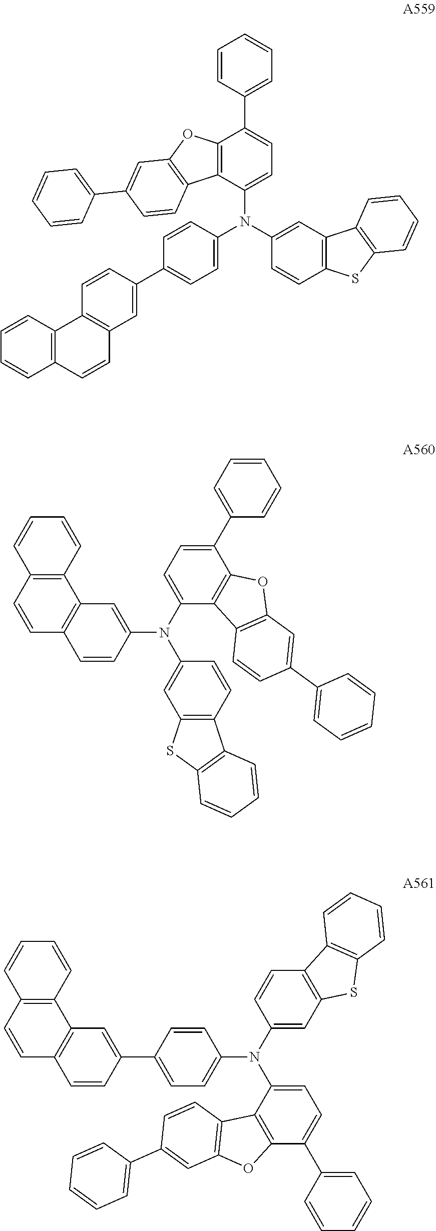 [SMILES: c1ccc(-c2ccc3c(c2)oc2c(-c4ccccc4)ccc(N(c4ccc(-c5ccc6c(ccc7ccccc76)c5)cc4)c4ccc5sc6ccccc6c5c4)c23)cc1 ; c1ccc(-c2ccc3c(c2)oc2c(-c4ccccc4)ccc(N(c4ccc5c(c4)sc4ccccc45)c4ccc5ccc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5c(c4)sc4ccccc45)c23)cc1]

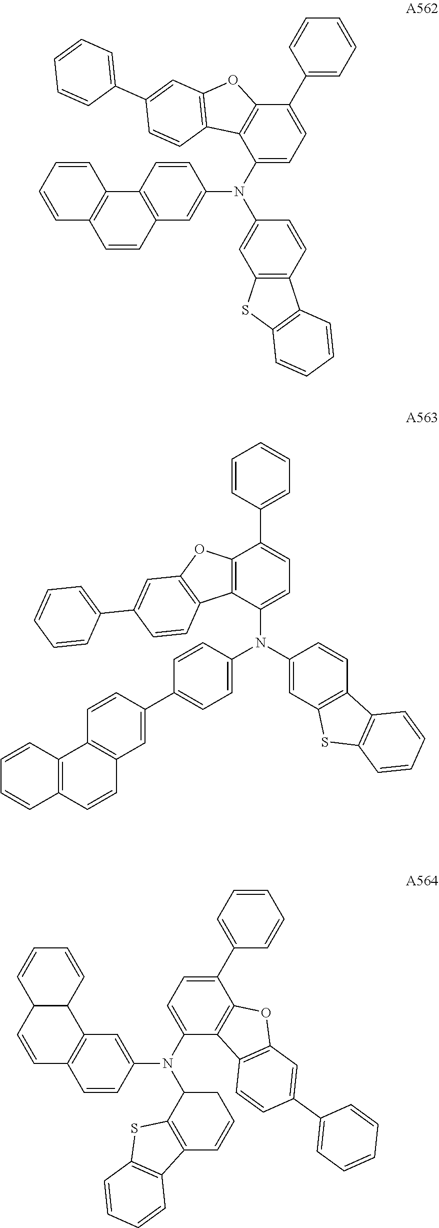 [SMILES: c1ccc(-c2ccc3c(c2)oc2c(-c4ccccc4)ccc(N(c4ccc5c(ccc6ccccc65)c4)c4ccc5c(c4)sc4ccccc45)c23)cc1 ; c1ccc(-c2ccc3c(c2)oc2c(-c4ccccc4)ccc(N(c4ccc(-c5ccc6c(ccc7ccccc76)c5)cc4)c4ccc5c(c4)sc4ccccc45)c23)cc1 ; C1=CC2C=Cc3ccc(N(c4ccc(-c5ccccc5)c5oc6cc(-c7ccccc7)ccc6c45)C4CC=Cc5c4sc4ccccc54)cc3C2C=C1]

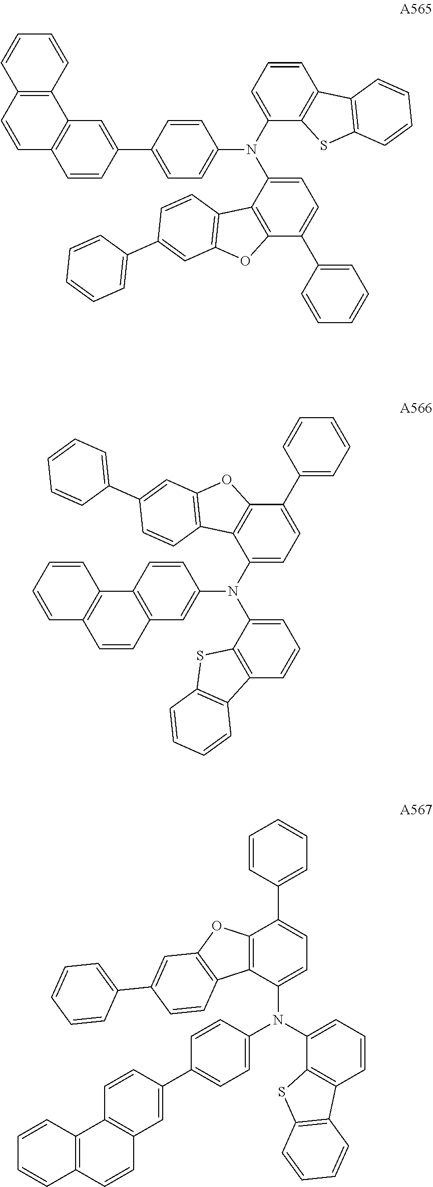 [SMILES: c1ccc(-c2ccc3c(c2)oc2c(-c4ccccc4)ccc(N(c4ccc(-c5ccc6ccc7ccccc7c6c5)cc4)c4cccc5c4sc4ccccc45)c23)cc1 ; c1ccc(-c2ccc3c(c2)oc2c(-c4ccccc4)ccc(N(c4ccc5c(ccc6ccccc65)c4)c4cccc5c4sc4ccccc45)c23)cc1 ; c1ccc(-c2ccc3c(c2)oc2c(-c4ccccc4)ccc(N(c4ccc(-c5ccc6c(ccc7ccccc76)c5)cc4)c4cccc5c4sc4ccccc45)c23)cc1]

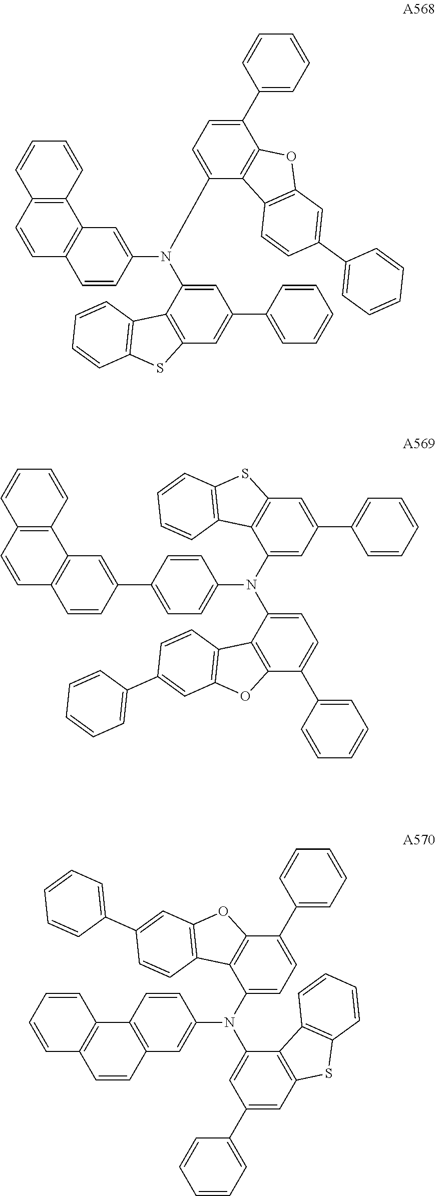 [SMILES: c1ccc(-c2ccc3c(c2)oc2c(-c4ccccc4)ccc(N(c4ccc5ccc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc(-c5ccc6ccc7ccccc7c6c5)cc4)c4cc(-c5ccccc5)cc5sc6ccccc6c45)c23)cc1 ; c1ccc(-c2ccc3c(c2)oc2c(-c4ccccc4)ccc(N(c4ccc5c(ccc6ccccc65)c4)c4cc(-c5ccccc5)cc5sc6ccccc6c45)c23)cc1]

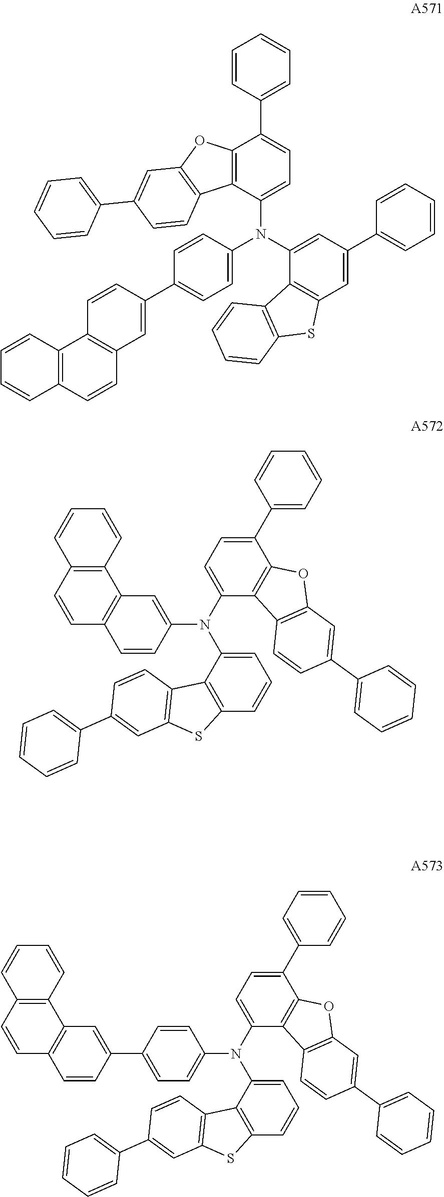 [SMILES: c1ccc(-c2ccc3c(c2)oc2c(-c4ccccc4)ccc(N(c4ccc(-c5ccc6c(ccc7ccccc76)c5)cc4)c4cc(-c5ccccc5)cc5sc6ccccc6c45)c23)cc1 ; c1ccc(-c2ccc3c(c2)oc2c(-c4ccccc4)ccc(N(c4ccc5ccc6ccccc6c5c4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6ccc7ccccc7c6c5)cc4)c4cccc5sc6cc(-c7ccccc7)ccc6c45)c23)cc1]

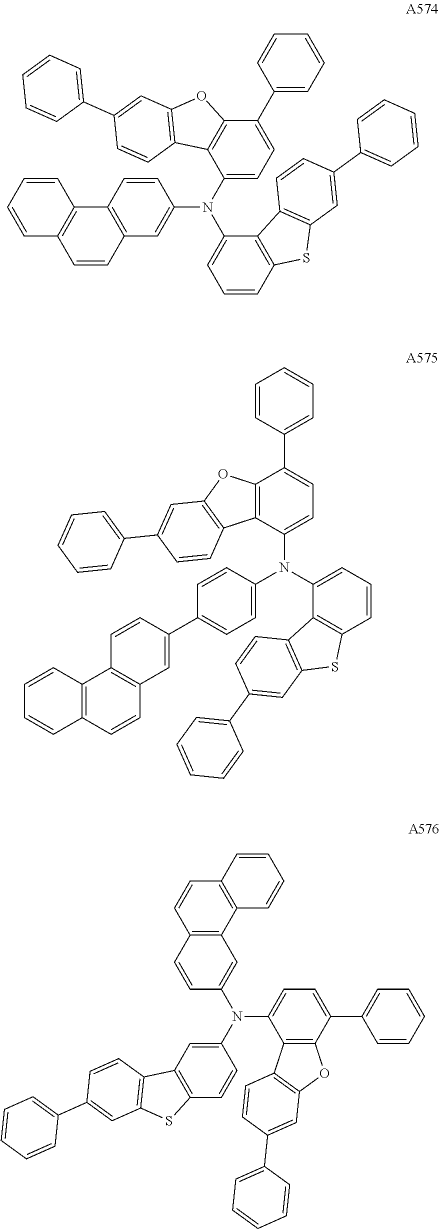 [SMILES: c1ccc(-c2ccc3c(c2)oc2c(-c4ccccc4)ccc(N(c4ccc5c(ccc6ccccc65)c4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5sc6cc(-c7ccccc7)ccc6c45)c23)cc1 ; c1ccc(-c2ccc3c(c2)oc2c(-c4ccccc4)ccc(N(c4ccc5ccc6ccccc6c5c4)c4ccc5sc6cc(-c7ccccc7)ccc6c5c4)c23)cc1]

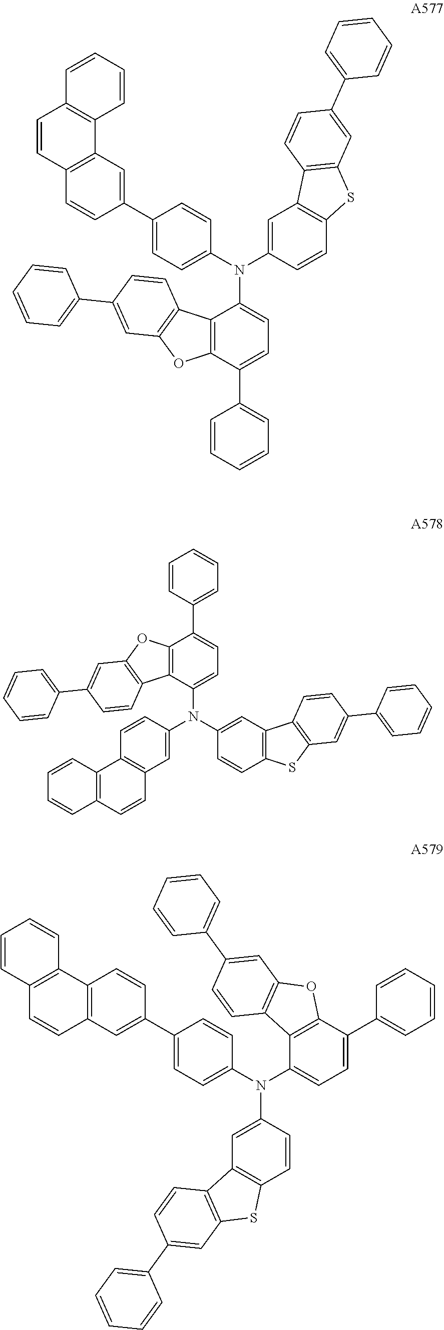 [SMILES: c1ccc(-c2ccc3c(c2)oc2c(-c4ccccc4)ccc(N(c4ccc(-c5ccc6ccc7ccccc7c6c5)cc4)c4ccc5sc6cc(-c7ccccc7)ccc6c5c4)c23)cc1 ; c1ccc(-c2ccc3c(c2)oc2c(-c4ccccc4)ccc(N(c4ccc5c(ccc6ccccc65)c4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6c(ccc7ccccc76)c5)cc4)c4ccc5sc6cc(-c7ccccc7)ccc6c5c4)c23)cc1]

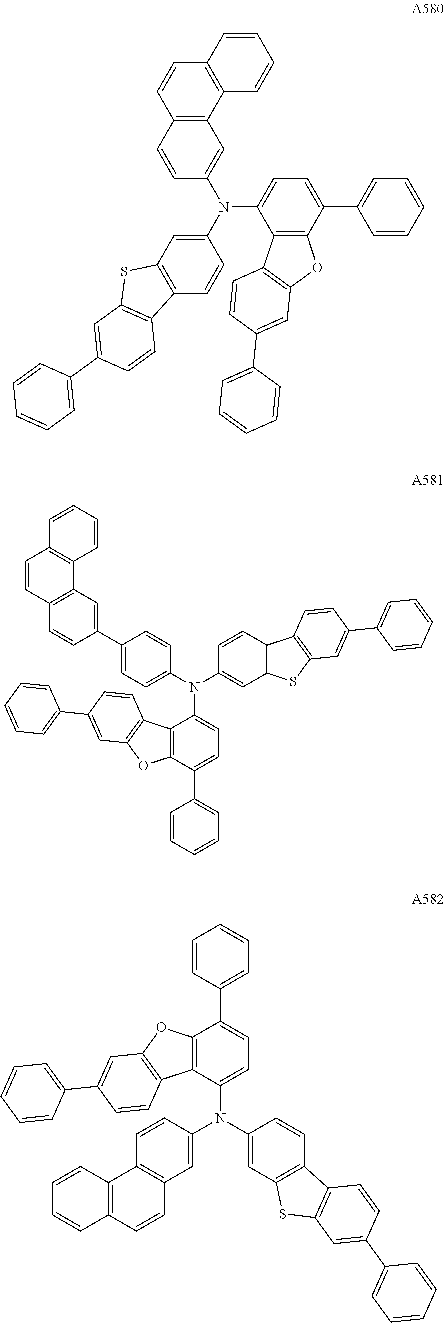 [SMILES: c1ccc(-c2ccc3c(c2)oc2c(-c4ccccc4)ccc(N(c4ccc5c(c4)sc4cc(-c6ccccc6)ccc45)c4ccc5ccc6ccccc6c5c4)c23)cc1 ; C1=CC2c3ccc(-c4ccccc4)cc3SC2C=C1N(c1ccc(-c2ccc3ccc4ccccc4c3c2)cc1)c1ccc(-c2ccccc2)c2oc3cc(-c4ccccc4)ccc3c12 ; c1ccc(-c2ccc3c(c2)oc2c(-c4ccccc4)ccc(N(c4ccc5c(ccc6ccccc65)c4)c4ccc5c(c4)sc4cc(-c6ccccc6)ccc45)c23)cc1]

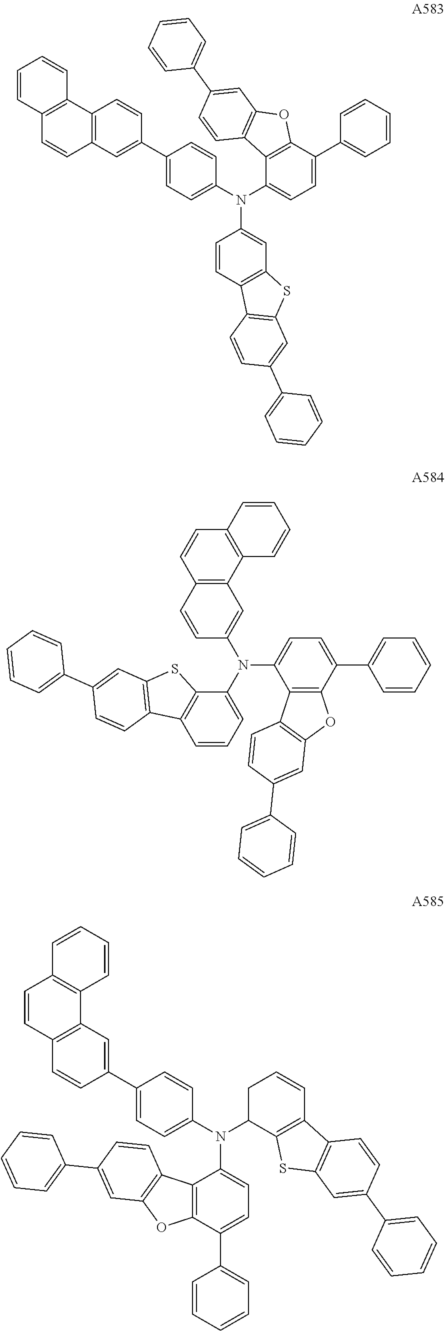 [SMILES: c1ccc(-c2ccc3c(c2)oc2c(-c4ccccc4)ccc(N(c4ccc(-c5ccc6c(ccc7ccccc76)c5)cc4)c4ccc5c(c4)sc4cc(-c6ccccc6)ccc45)c23)cc1 ; c1ccc(-c2ccc3c(c2)oc2c(-c4ccccc4)ccc(N(c4ccc5ccc6ccccc6c5c4)c4cccc5c4sc4cc(-c6ccccc6)ccc45)c23)cc1 ; C1=Cc2c(sc3cc(-c4ccccc4)ccc23)C(N(c2ccc(-c3ccc4ccc5ccccc5c4c3)cc2)c2ccc(-c3ccccc3)c3oc4cc(-c5ccccc5)ccc4c23)C1]

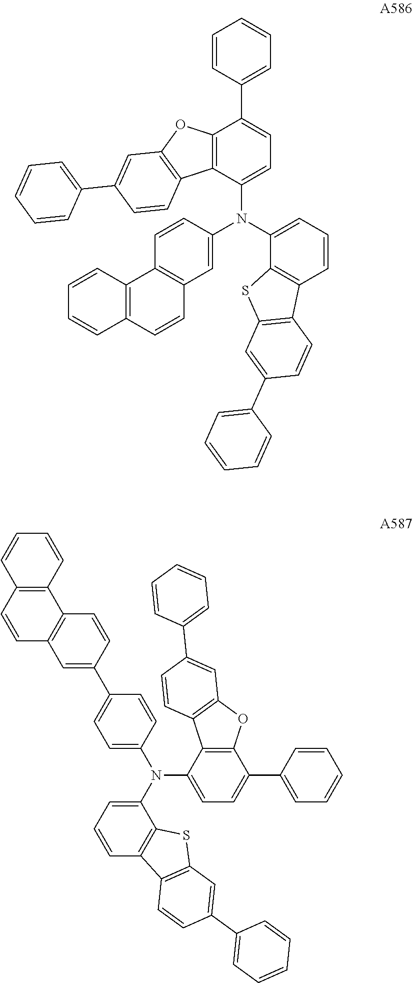 [SMILES: c1ccc(-c2ccc3c(c2)oc2c(-c4ccccc4)ccc(N(c4ccc5c(ccc6ccccc65)c4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6c(ccc7ccccc76)c5)cc4)c4cccc5c4sc4cc(-c6ccccc6)ccc45)c23)cc1]

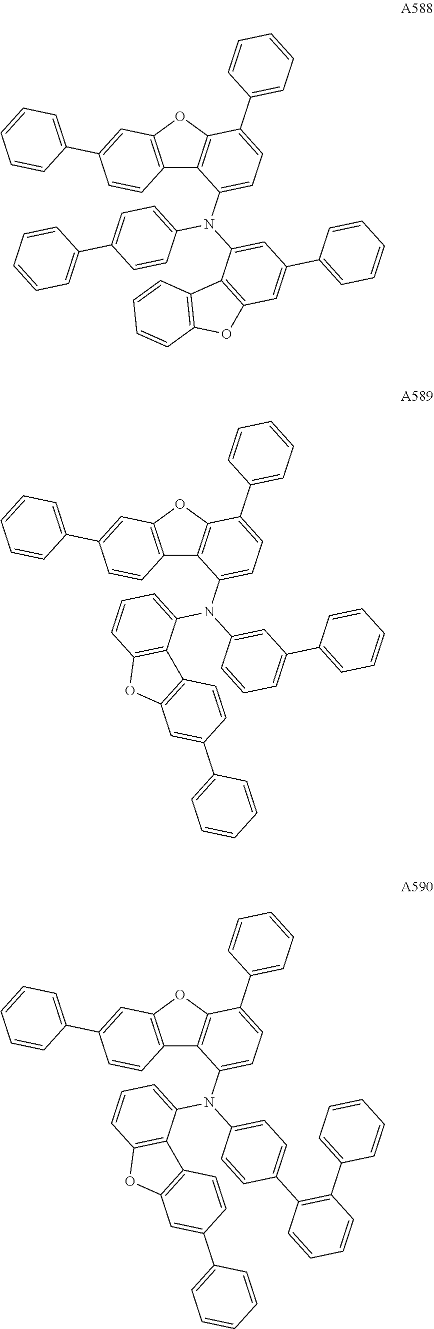 [SMILES: c1ccc(-c2ccc(N(c3cc(-c4ccccc4)cc4oc5ccccc5c34)c3ccc(-c4ccccc4)c4oc5cc(-c6ccccc6)ccc5c34)cc2)cc1 ; c1ccc(-c2cccc(N(c3cccc4oc5cc(-c6ccccc6)ccc5c34)c3ccc(-c4ccccc4)c4oc5cc(-c6ccccc6)ccc5c34)c2)cc1 ; c1ccc(-c2ccc3c(c2)oc2cccc(N(c4ccc(-c5ccccc5-c5ccccc5)cc4)c4ccc(-c5ccccc5)c5oc6cc(-c7ccccc7)ccc6c45)c23)cc1]

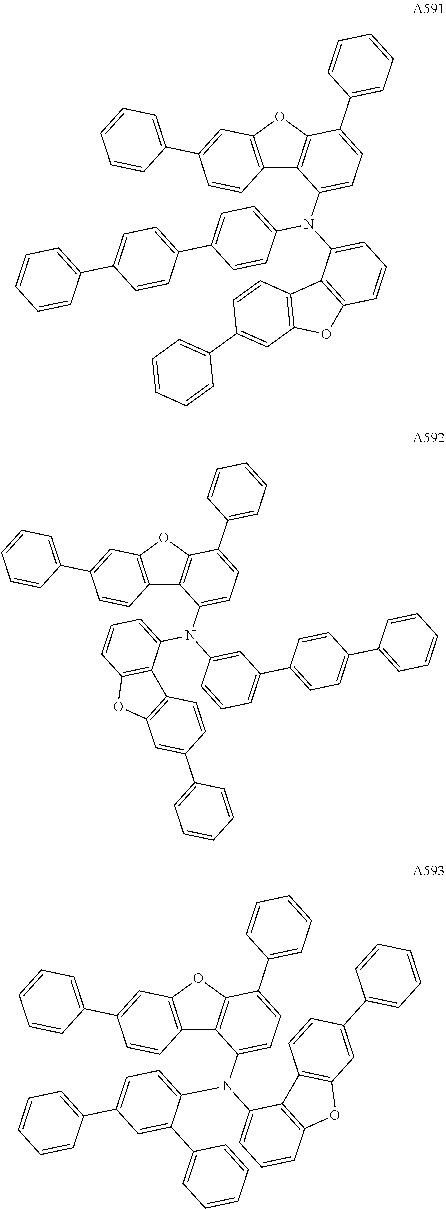 [SMILES: c1ccc(-c2ccc(-c3ccc(N(c4cccc5oc6cc(-c7ccccc7)ccc6c45)c4ccc(-c5ccccc5)c5oc6cc(-c7ccccc7)ccc6c45)cc3)cc2)cc1 ; c1ccc(-c2ccc(-c3cccc(N(c4cccc5oc6cc(-c7ccccc7)ccc6c45)c4ccc(-c5ccccc5)c5oc6cc(-c7ccccc7)ccc6c45)c3)cc2)cc1 ; c1ccc(-c2ccc(N(c3cccc4oc5cc(-c6ccccc6)ccc5c34)c3ccc(-c4ccccc4)c4oc5cc(-c6ccccc6)ccc5c34)c(-c3ccccc3)c2)cc1]

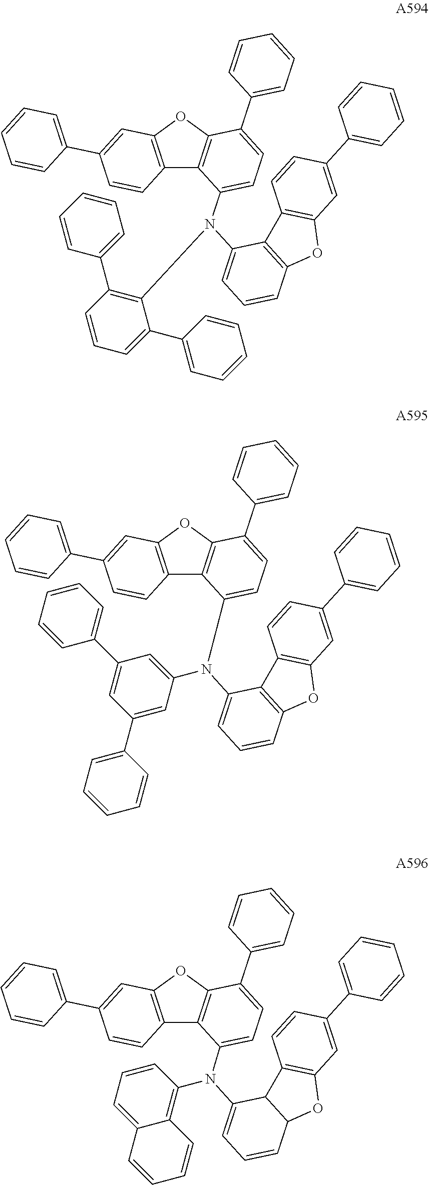 [SMILES: c1ccc(-c2ccc3c(c2)oc2cccc(N(c4c(-c5ccccc5)cccc4-c4ccccc4)c4ccc(-c5ccccc5)c5oc6cc(-c7ccccc7)ccc6c45)c23)cc1 ; c1ccc(-c2cc(-c3ccccc3)cc(N(c3cccc4oc5cc(-c6ccccc6)ccc5c34)c3ccc(-c4ccccc4)c4oc5cc(-c6ccccc6)ccc5c34)c2)cc1 ; C1=CC2Oc3cc(-c4ccccc4)ccc3C2C(N(c2cccc3ccccc23)c2ccc(-c3ccccc3)c3oc4cc(-c5ccccc5)ccc4c23)=C1]

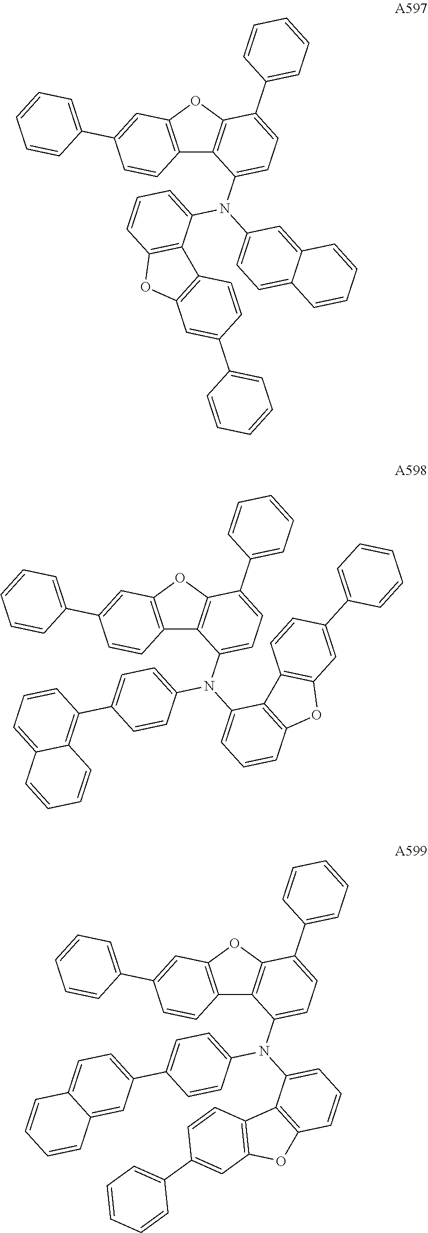 [SMILES: c1ccc(-c2ccc3c(c2)oc2cccc(N(c4ccc5ccccc5c4)c4ccc(-c5ccccc5)c5oc6cc(-c7ccccc7)ccc6c45)c23)cc1 ; c1ccc(-c2ccc3c(c2)oc2cccc(N(c4ccc(-c5cccc6ccccc56)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6ccccc6c5)cc4)c4ccc(-c5ccccc5)c5oc6cc(-c7ccccc7)ccc6c45)c23)cc1]

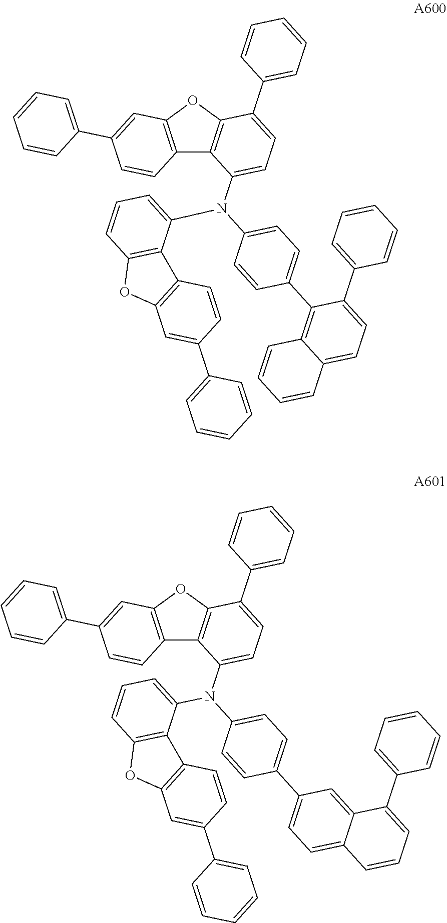 [SMILES: c1ccc(-c2ccc3c(c2)oc2cccc(N(c4ccc(-c5c(-c6ccccc6)ccc6ccccc56)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6cccc(-c7ccccc7)c6c5)cc4)c4ccc(-c5ccccc5)c5oc6cc(-c7ccccc7)ccc6c45)c23)cc1]

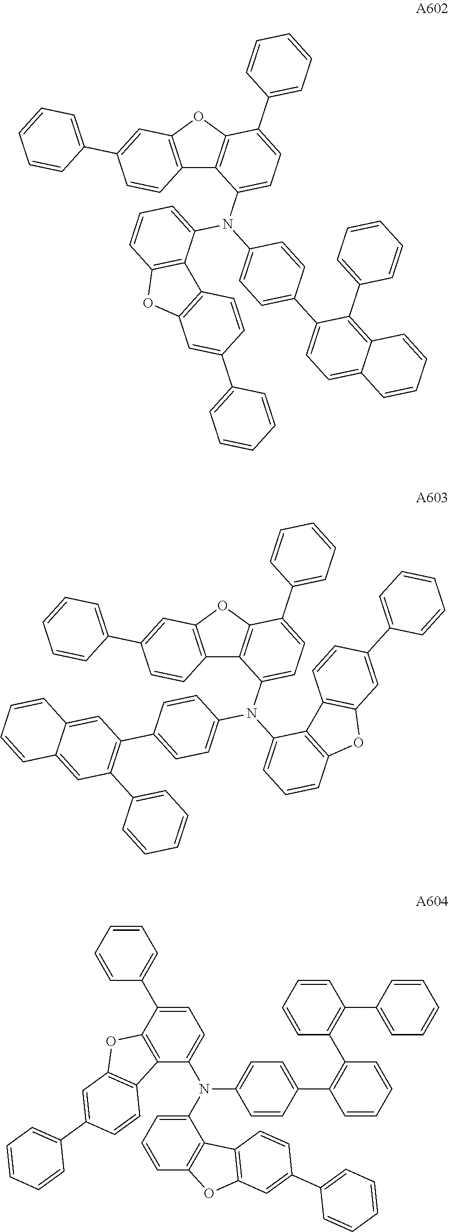 [SMILES: c1ccc(-c2ccc3c(c2)oc2cccc(N(c4ccc(-c5ccc6ccccc6c5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5cc6ccccc6cc5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccccc5-c5ccccc5-c5ccccc5)cc4)c4ccc(-c5ccccc5)c5oc6cc(-c7ccccc7)ccc6c45)c23)cc1]

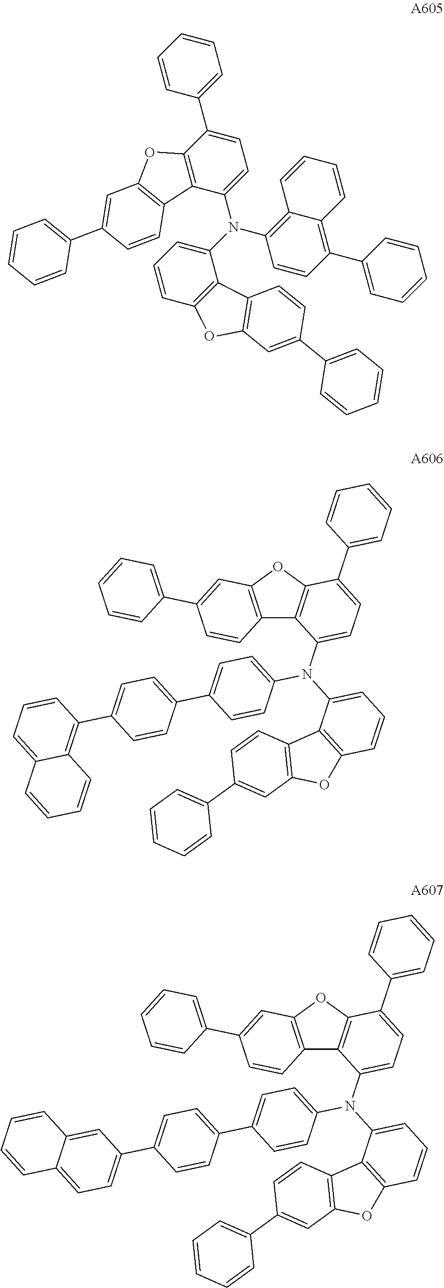 [SMILES: c1ccc(-c2ccc3c(c2)oc2cccc(N(c4ccc(-c5ccccc5)c5ccccc45)c4ccc(-c5ccccc5)c5oc6cc(-c7ccccc7)ccc6c45)c23)cc1 ; c1ccc(-c2ccc3c(c2)oc2cccc(N(c4ccc(-c5ccc(-c6cccc7ccccc67)cc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5oc6cc(-c7ccccc7)ccc6c45)c23)cc1]

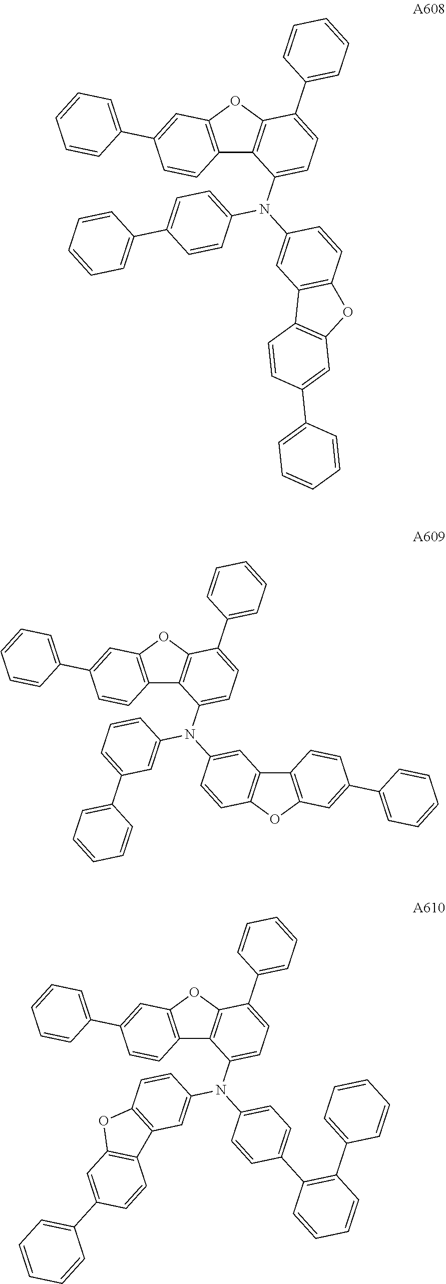 [SMILES: c1ccc(-c2ccc(N(c3ccc4oc5cc(-c6ccccc6)ccc5c4c3)c3ccc(-c4ccccc4)c4oc5cc(-c6ccccc6)ccc5c34)cc2)cc1 ; c1ccc(-c2cccc(N(c3ccc4oc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2ccc(N(c4ccc(-c5ccccc5-c5ccccc5)cc4)c4ccc(-c5ccccc5)c5oc6cc(-c7ccccc7)ccc6c45)cc23)cc1]

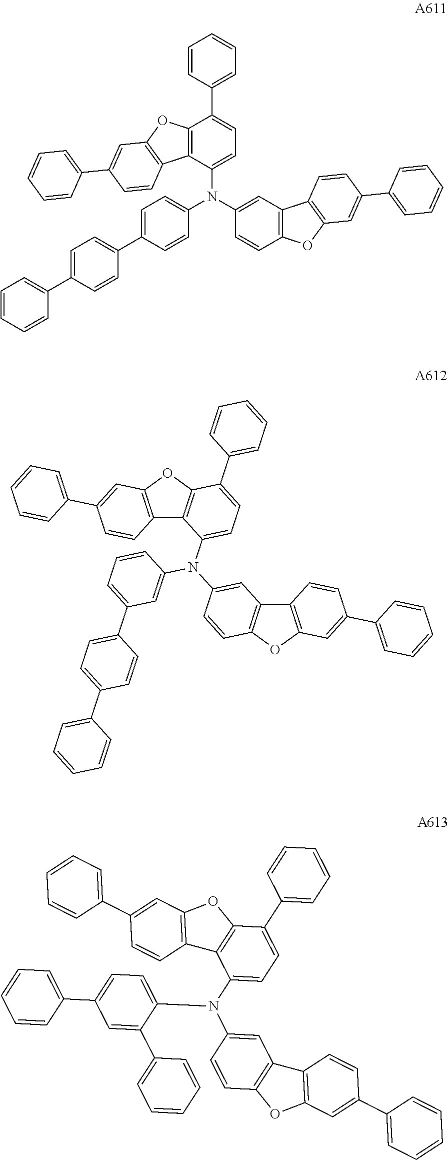 [SMILES: c1ccc(-c2ccc(-c3ccc(N(c4ccc5oc6cc(-c7ccccc7)ccc6c5c4)c4ccc(-c5ccccc5)c5oc6cc(-c7ccccc7)ccc6c45)cc3)cc2)cc1 ; c1ccc(-c2ccc(-c3cccc(N(c4ccc5oc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4oc5cc(-c6ccccc6)ccc5c4c3)c3ccc(-c4ccccc4)c4oc5cc(-c6ccccc6)ccc5c34)c(-c3ccccc3)c2)cc1]

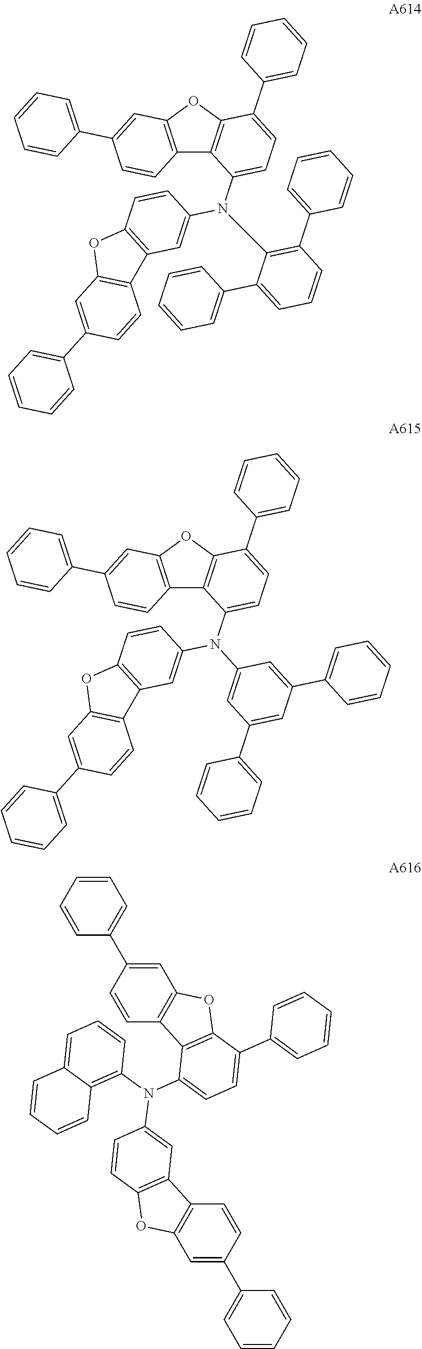

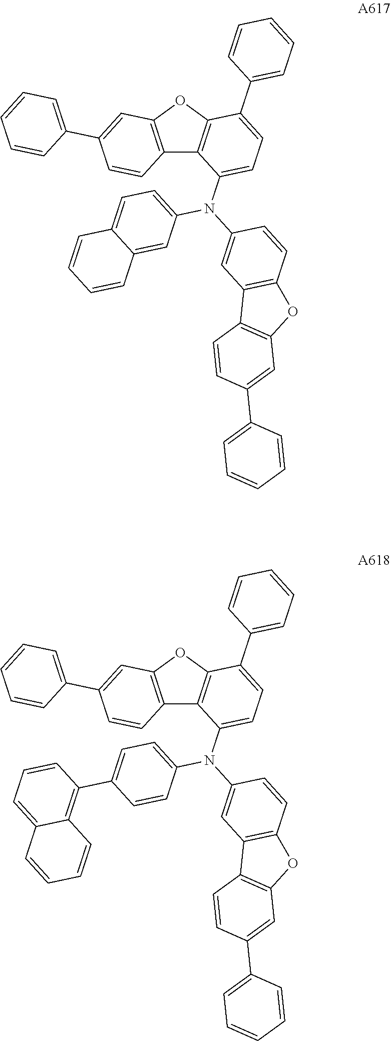 [SMILES: c1ccc(-c2ccc3c(c2)oc2ccc(N(c4ccc5ccccc5c4)c4ccc(-c5ccccc5)c5oc6cc(-c7ccccc7)ccc6c45)cc23)cc1 ; c1ccc(-c2ccc3c(c2)oc2ccc(N(c4ccc(-c5cccc6ccccc56)cc4)c4ccc(-c5ccccc5)c5oc6cc(-c7ccccc7)ccc6c45)cc23)cc1]

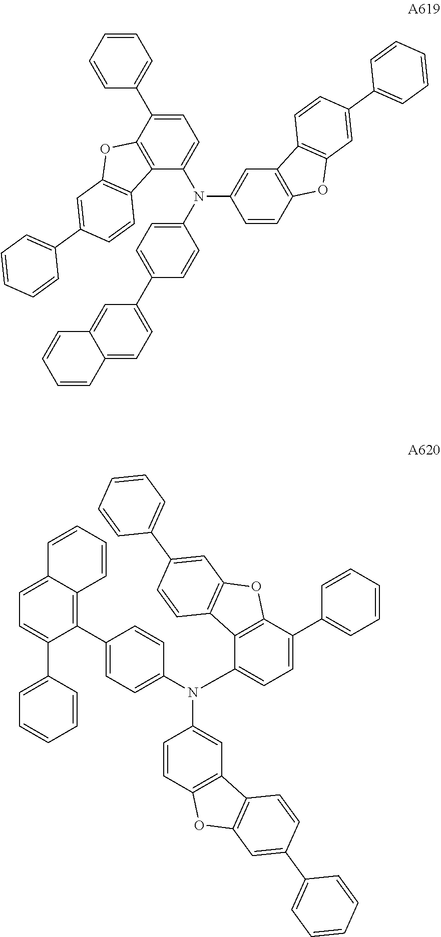 [SMILES: c1ccc(-c2ccc3c(c2)oc2ccc(N(c4ccc(-c5ccc6ccccc6c5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5c(-c6ccccc6)ccc6ccccc56)cc4)c4ccc(-c5ccccc5)c5oc6cc(-c7ccccc7)ccc6c45)cc23)cc1]

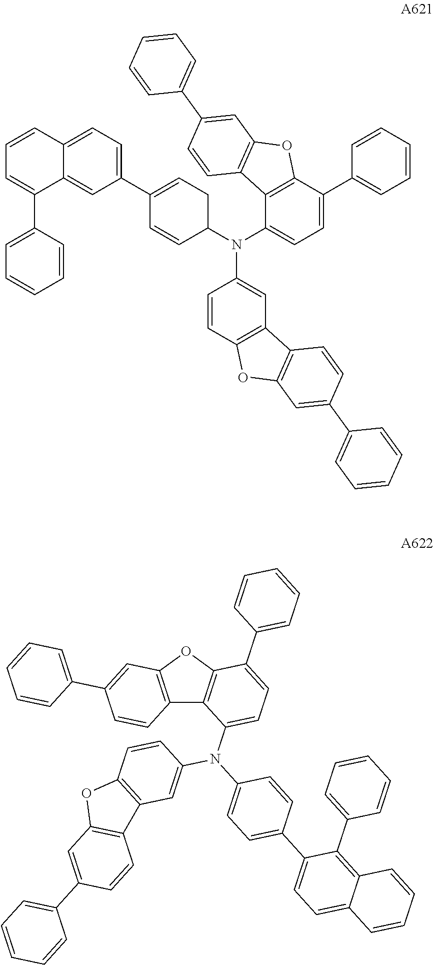 [SMILES: C1=CC(N(c2ccc3oc4cc(-c5ccccc5)ccc4c3c2)c2ccc(-c3ccccc3)c3oc4cc(-c5ccccc5)ccc4c23)CC=C1c1ccc2cccc(-c3ccccc3)c2c1 ; c1ccc(-c2ccc3c(c2)oc2ccc(N(c4ccc(-c5ccc6ccccc6c5-c5ccccc5)cc4)c4ccc(-c5ccccc5)c5oc6cc(-c7ccccc7)ccc6c45)cc23)cc1]

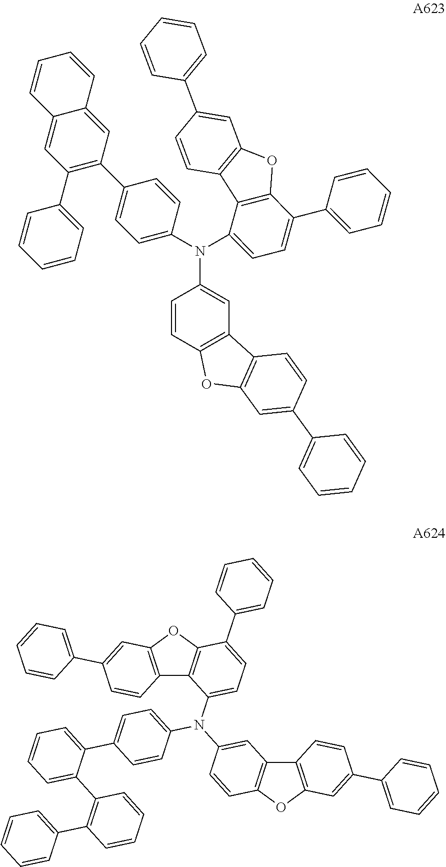 [SMILES: c1ccc(-c2ccc3c(c2)oc2ccc(N(c4ccc(-c5cc6ccccc6cc5-c5ccccc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccccc5-c5ccccc5-c5ccccc5)cc4)c4ccc(-c5ccccc5)c5oc6cc(-c7ccccc7)ccc6c45)cc23)cc1]

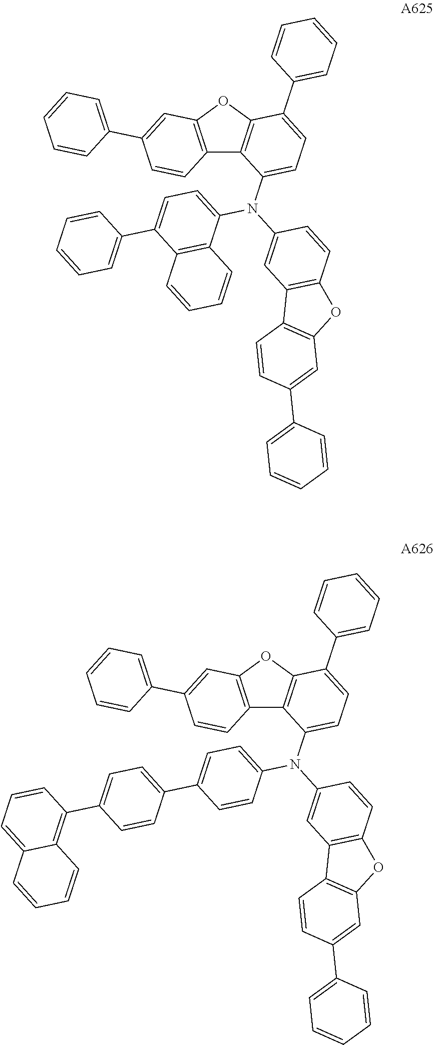 [SMILES: c1ccc(-c2ccc3c(c2)oc2ccc(N(c4ccc(-c5ccccc5)c5ccccc45)c4ccc(-c5ccccc5)c5oc6cc(-c7ccccc7)ccc6c45)cc23)cc1 ; c1ccc(-c2ccc3c(c2)oc2ccc(N(c4ccc(-c5ccc(-c6cccc7ccccc67)cc5)cc4)c4ccc(-c5ccccc5)c5oc6cc(-c7ccccc7)ccc6c45)cc23)cc1]

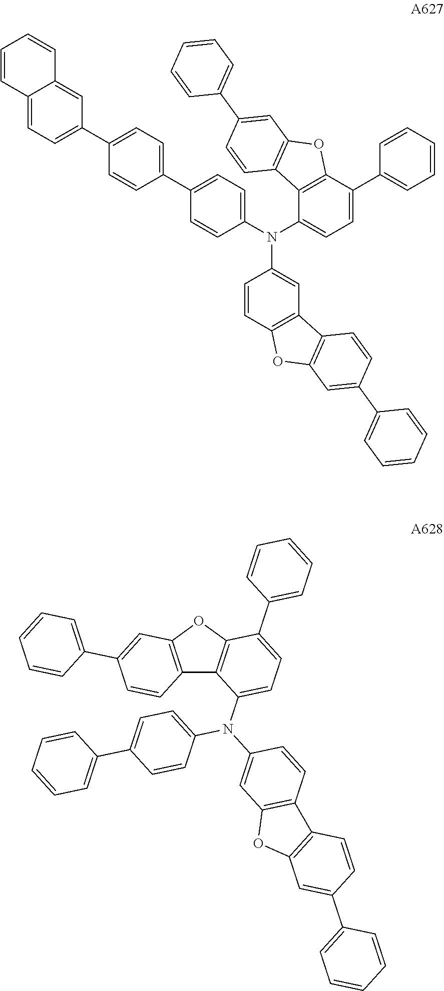 [SMILES: c1ccc(-c2ccc3c(c2)oc2ccc(N(c4ccc(-c5ccc(-c6ccc7ccccc7c6)cc5)cc4)c4ccc(-c5ccccc5)c5oc6cc(-c7ccccc7)ccc6c45)cc23)cc1 ; c1ccc(-c2ccc(N(c3ccc4c(c3)oc3cc(-c5ccccc5)ccc34)c3ccc(-c4ccccc4)c4oc5cc(-c6ccccc6)ccc5c34)cc2)cc1]

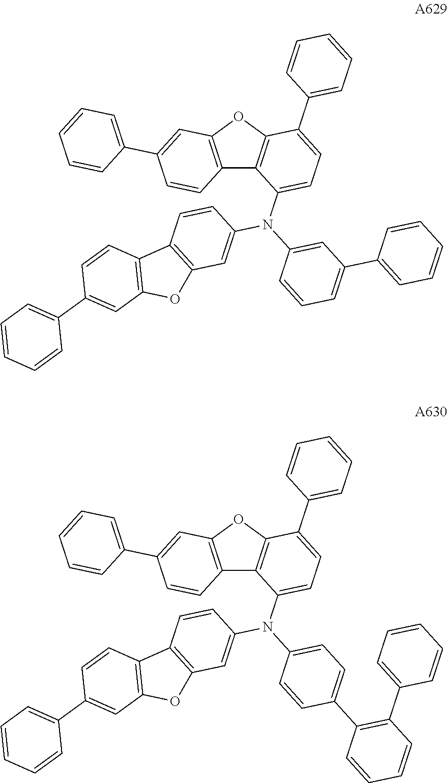 [SMILES: c1ccc(-c2cccc(N(c3ccc4c(c3)oc3cc(-c5ccccc5)ccc34)c3ccc(-c4ccccc4)c4oc5cc(-c6ccccc6)ccc5c34)c2)cc1 ; c1ccc(-c2ccc3c(c2)oc2cc(N(c4ccc(-c5ccccc5-c5ccccc5)cc4)c4ccc(-c5ccccc5)c5oc6cc(-c7ccccc7)ccc6c45)ccc23)cc1]

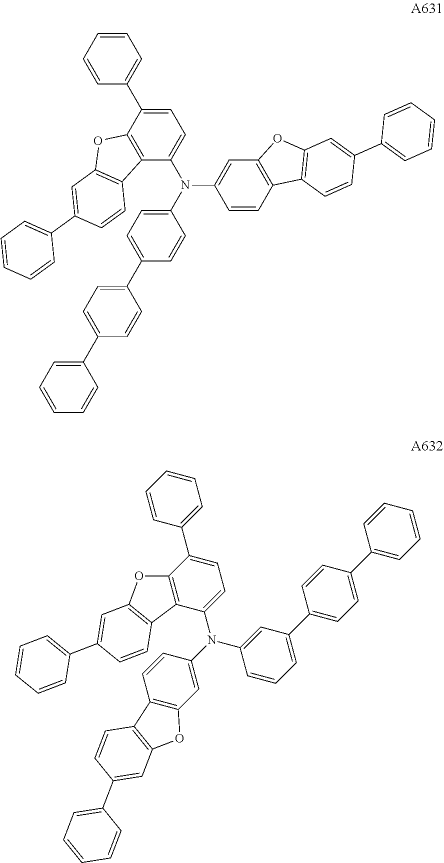 [SMILES: c1ccc(-c2ccc(-c3ccc(N(c4ccc5c(c4)oc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)oc4cc(-c6ccccc6)ccc45)c4ccc(-c5ccccc5)c5oc6cc(-c7ccccc7)ccc6c45)c3)cc2)cc1]

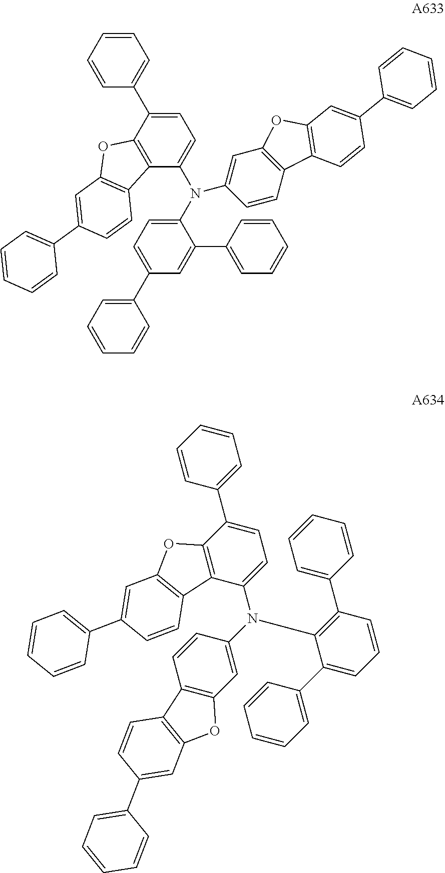 [SMILES: c1ccc(-c2ccc(N(c3ccc4c(c3)oc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2cc(N(c4c(-c5ccccc5)cccc4-c4ccccc4)c4ccc(-c5ccccc5)c5oc6cc(-c7ccccc7)ccc6c45)ccc23)cc1]

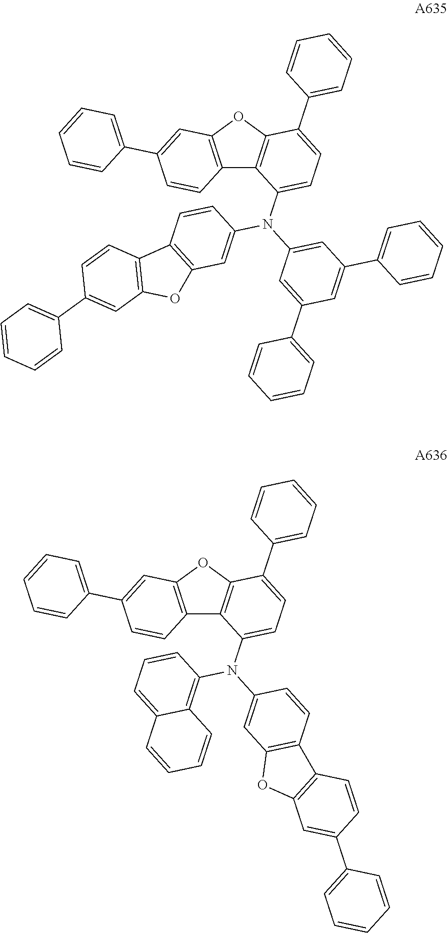 [SMILES: c1ccc(-c2cc(-c3ccccc3)cc(N(c3ccc4c(c3)oc3cc(-c5ccccc5)ccc34)c3ccc(-c4ccccc4)c4oc5cc(-c6ccccc6)ccc5c34)c2)cc1 ; c1ccc(-c2ccc3c(c2)oc2cc(N(c4cccc5ccccc45)c4ccc(-c5ccccc5)c5oc6cc(-c7ccccc7)ccc6c45)ccc23)cc1]

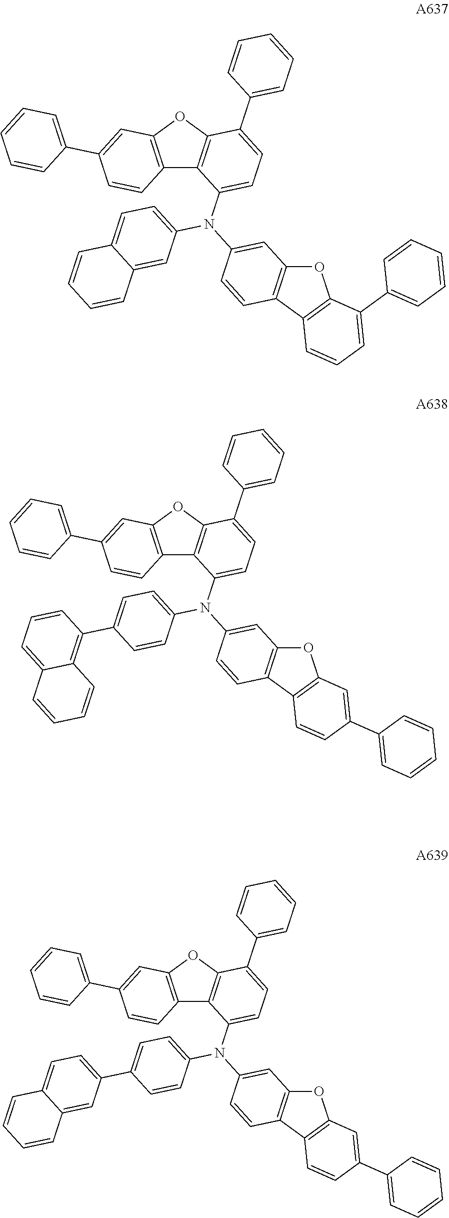 [SMILES: c1ccc(-c2ccc3c(c2)oc2c(-c4ccccc4)ccc(N(c4ccc5ccccc5c4)c4ccc5c(c4)oc4c(-c6ccccc6)cccc45)c23)cc1 ; c1ccc(-c2ccc3c(c2)oc2cc(N(c4ccc(-c5cccc6ccccc56)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6ccccc6c5)cc4)c4ccc(-c5ccccc5)c5oc6cc(-c7ccccc7)ccc6c45)ccc23)cc1]

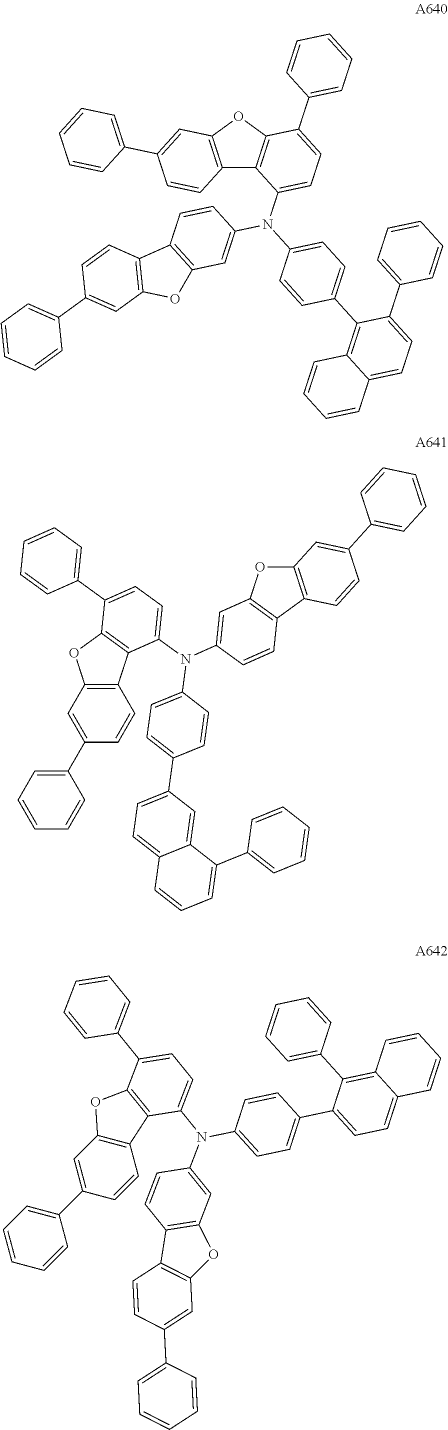 [SMILES: c1ccc(-c2ccc3c(c2)oc2cc(N(c4ccc(-c5c(-c6ccccc6)ccc6ccccc56)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6cccc(-c7ccccc7)c6c5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6ccccc6c5-c5ccccc5)cc4)c4ccc(-c5ccccc5)c5oc6cc(-c7ccccc7)ccc6c45)ccc23)cc1]

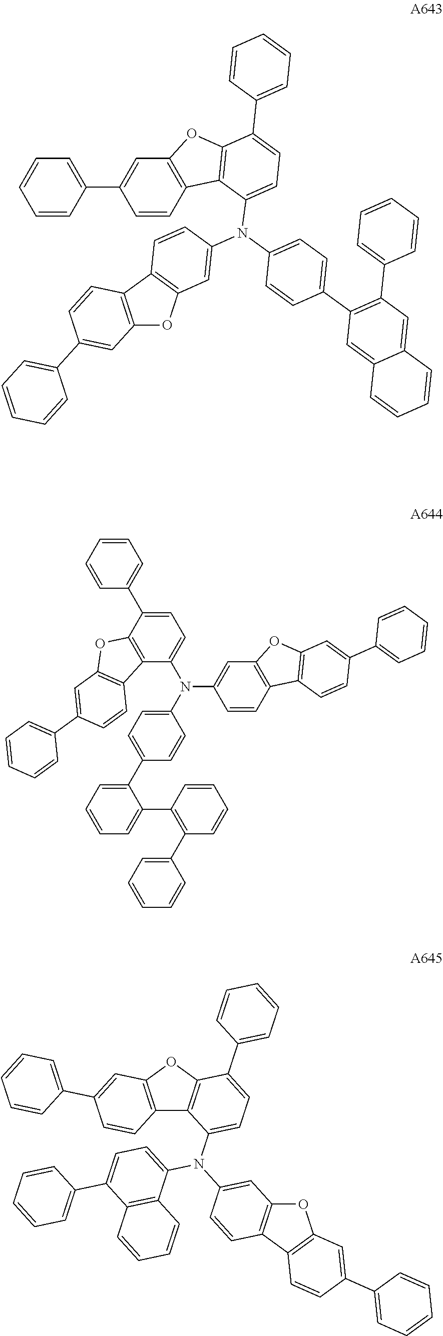 [SMILES: c1ccc(-c2ccc3c(c2)oc2cc(N(c4ccc(-c5cc6ccccc6cc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5-c5ccccc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5)c5ccccc45)c4ccc(-c5ccccc5)c5oc6cc(-c7ccccc7)ccc6c45)ccc23)cc1]

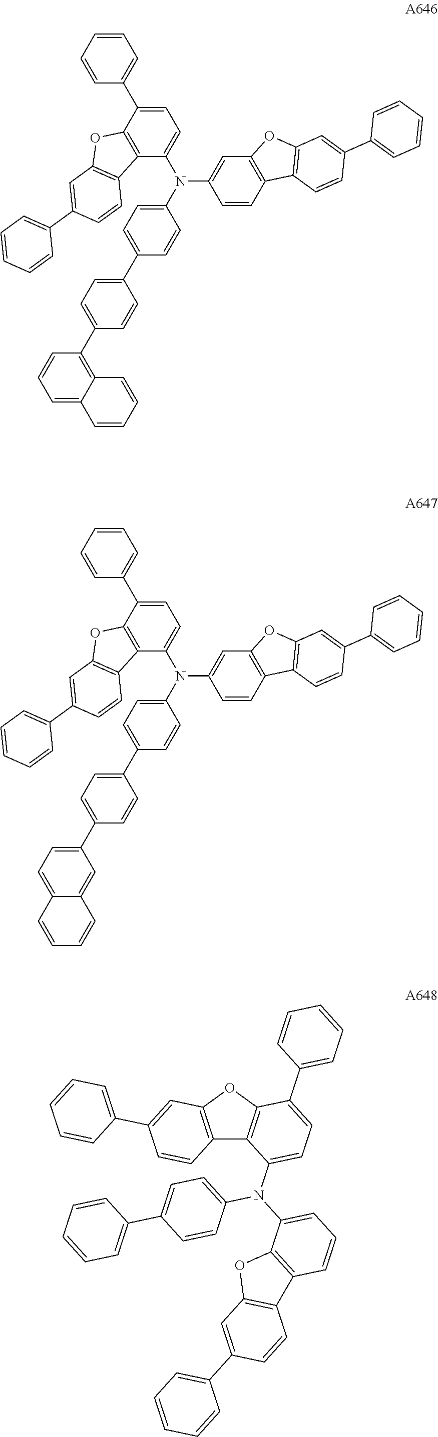 [SMILES: c1ccc(-c2ccc3c(c2)oc2cc(N(c4ccc(-c5ccc(-c6cccc7ccccc67)cc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5oc6cc(-c7ccccc7)ccc6c45)ccc23)cc1 ; c1ccc(-c2ccc(N(c3cccc4c3oc3cc(-c5ccccc5)ccc34)c3ccc(-c4ccccc4)c4oc5cc(-c6ccccc6)ccc5c34)cc2)cc1]

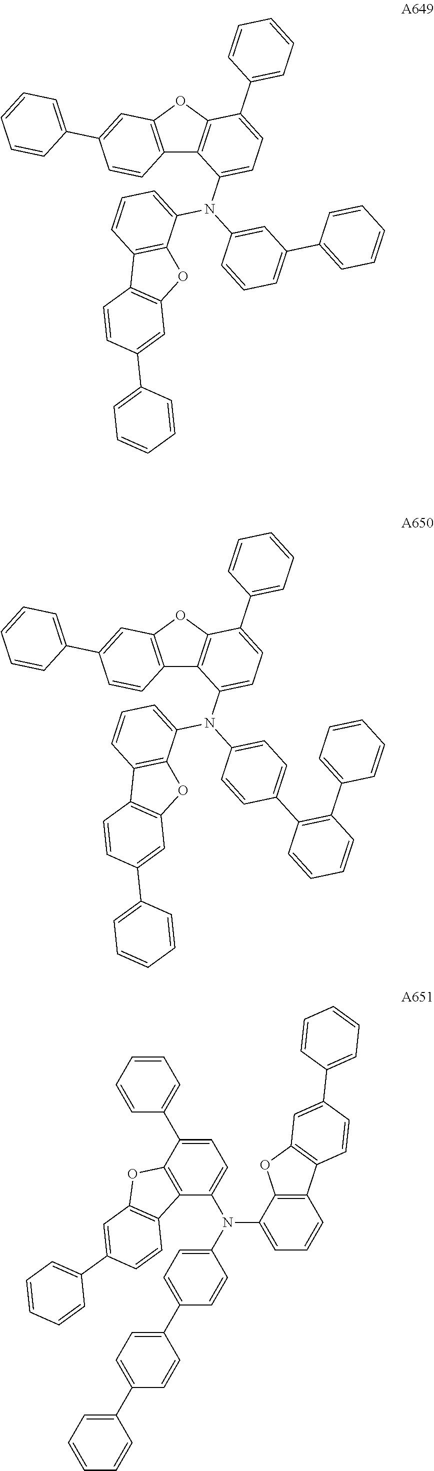 [SMILES: c1ccc(-c2cccc(N(c3cccc4c3oc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(N(c4ccc(-c5ccccc5-c5ccccc5)cc4)c4ccc(-c5ccccc5)c5oc6cc(-c7ccccc7)ccc6c45)cccc23)cc1 ; c1ccc(-c2ccc(-c3ccc(N(c4cccc5c4oc4cc(-c6ccccc6)ccc45)c4ccc(-c5ccccc5)c5oc6cc(-c7ccccc7)ccc6c45)cc3)cc2)cc1]

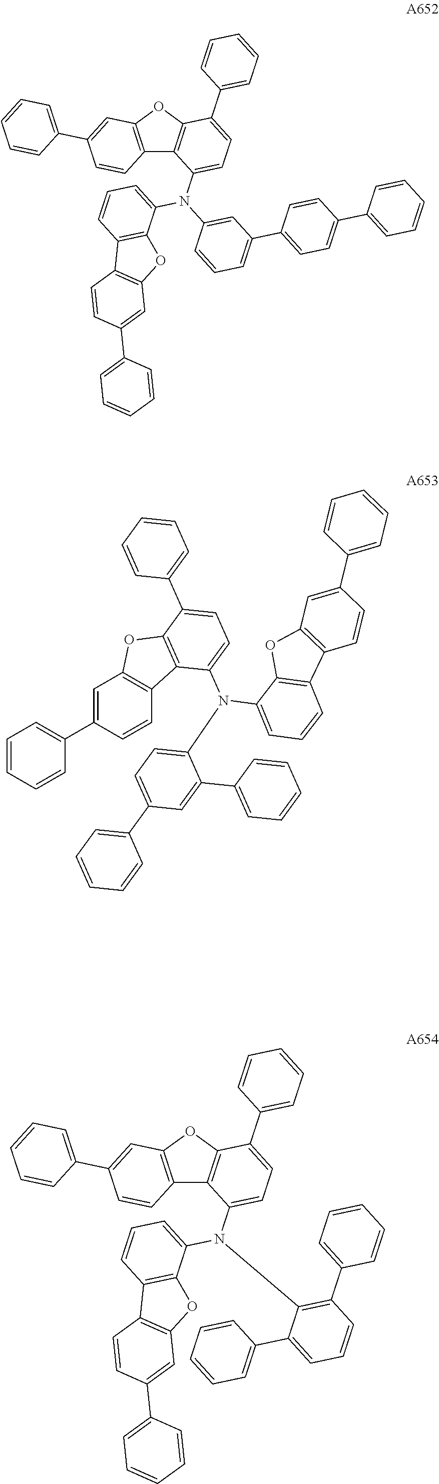 [SMILES: c1ccc(-c2ccc(-c3cccc(N(c4cccc5c4oc4cc(-c6ccccc6)ccc45)c4ccc(-c5ccccc5)c5oc6cc(-c7ccccc7)ccc6c45)c3)cc2)cc1 ; c1ccc(-c2ccc(N(c3cccc4c3oc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(N(c4c(-c5ccccc5)cccc4-c4ccccc4)c4ccc(-c5ccccc5)c5oc6cc(-c7ccccc7)ccc6c45)cccc23)cc1]

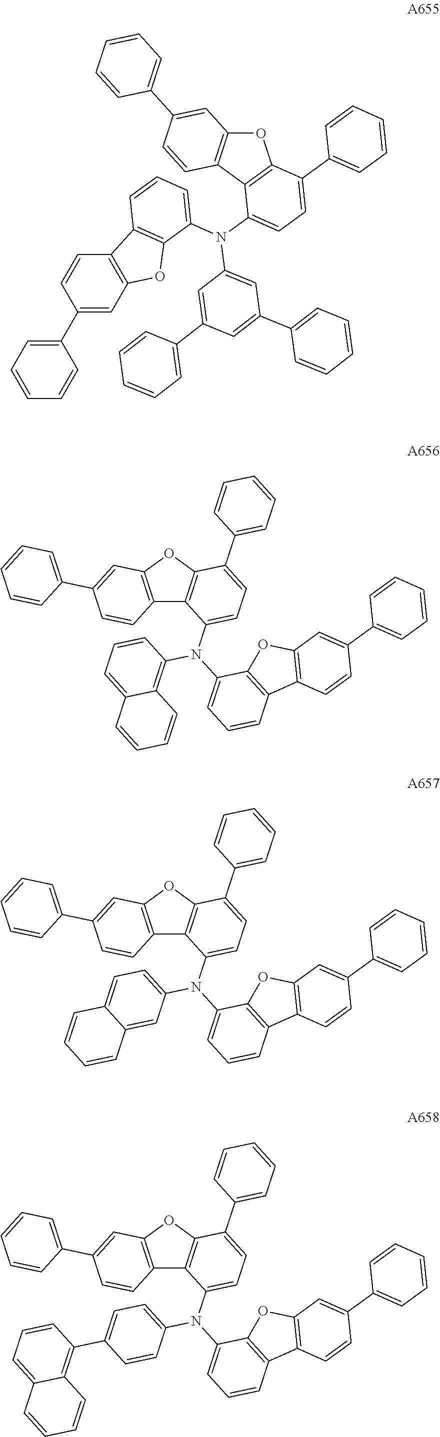 [SMILES: c1ccc(-c2cc(-c3ccccc3)cc(N(c3cccc4c3oc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(N(c4cccc5ccccc45)c4ccc(-c5ccccc5)c5oc6cc(-c7ccccc7)ccc6c45)cccc23)cc1 ; c1ccc(-c2ccc3c(c2)oc2c(N(c4ccc5ccccc5c4)c4ccc(-c5ccccc5)c5oc6cc(-c7ccccc7)ccc6c45)cccc23)cc1 ; c1ccc(-c2ccc3c(c2)oc2c(N(c4ccc(-c5cccc6ccccc56)cc4)c4ccc(-c5ccccc5)c5oc6cc(-c7ccccc7)ccc6c45)cccc23)cc1]

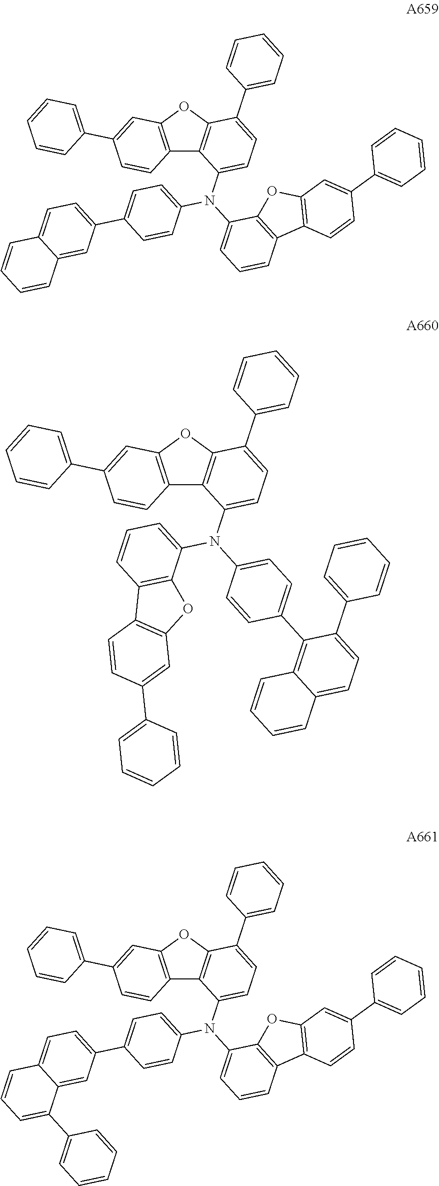 [SMILES: c1ccc(-c2ccc3c(c2)oc2c(N(c4ccc(-c5ccc6ccccc6c5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5c(-c6ccccc6)ccc6ccccc56)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6cccc(-c7ccccc7)c6c5)cc4)c4ccc(-c5ccccc5)c5oc6cc(-c7ccccc7)ccc6c45)cccc23)cc1]

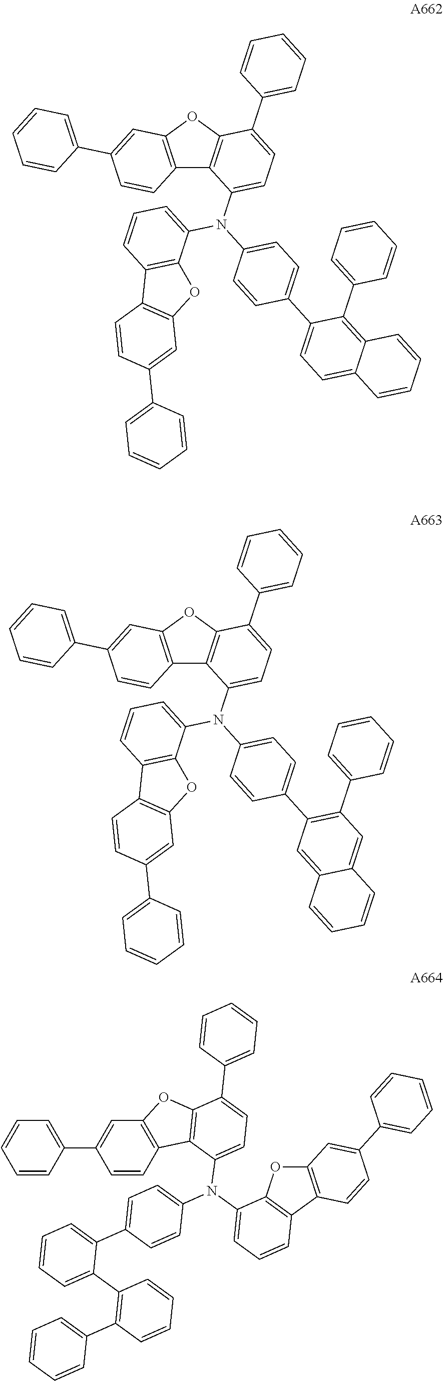 [SMILES: c1ccc(-c2ccc3c(c2)oc2c(N(c4ccc(-c5ccc6ccccc6c5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5cc6ccccc6cc5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccccc5-c5ccccc5-c5ccccc5)cc4)c4ccc(-c5ccccc5)c5oc6cc(-c7ccccc7)ccc6c45)cccc23)cc1]

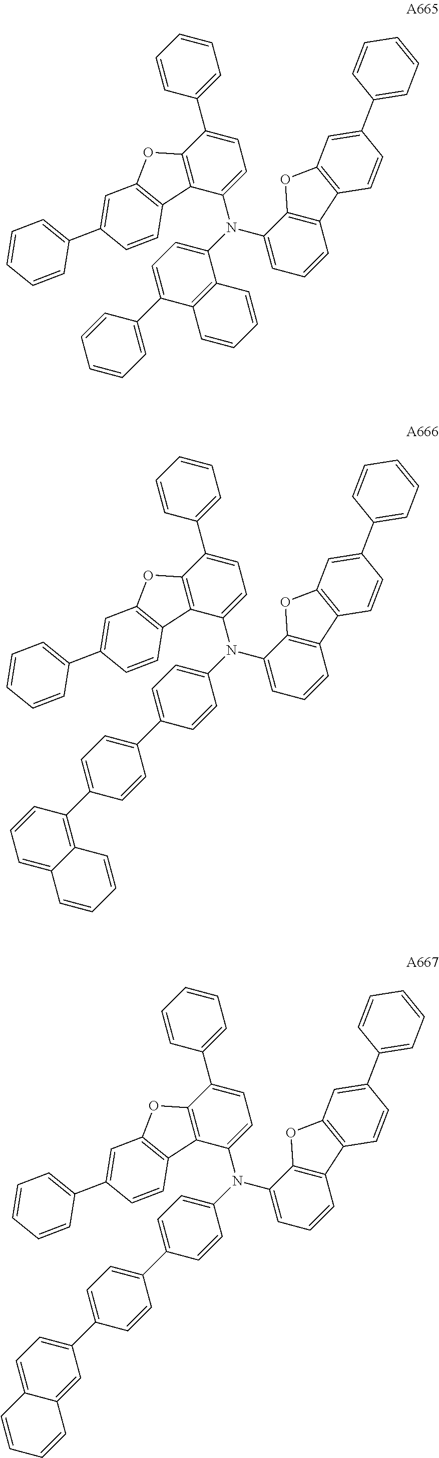 [SMILES: c1ccc(-c2ccc3c(c2)oc2c(N(c4ccc(-c5ccccc5)c5ccccc45)c4ccc(-c5ccccc5)c5oc6cc(-c7ccccc7)ccc6c45)cccc23)cc1 ; c1ccc(-c2ccc3c(c2)oc2c(N(c4ccc(-c5ccc(-c6cccc7ccccc67)cc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc(-c6ccc7ccccc7c6)cc5)cc4)c4ccc(-c5ccccc5)c5oc6cc(-c7ccccc7)ccc6c45)cccc23)cc1]

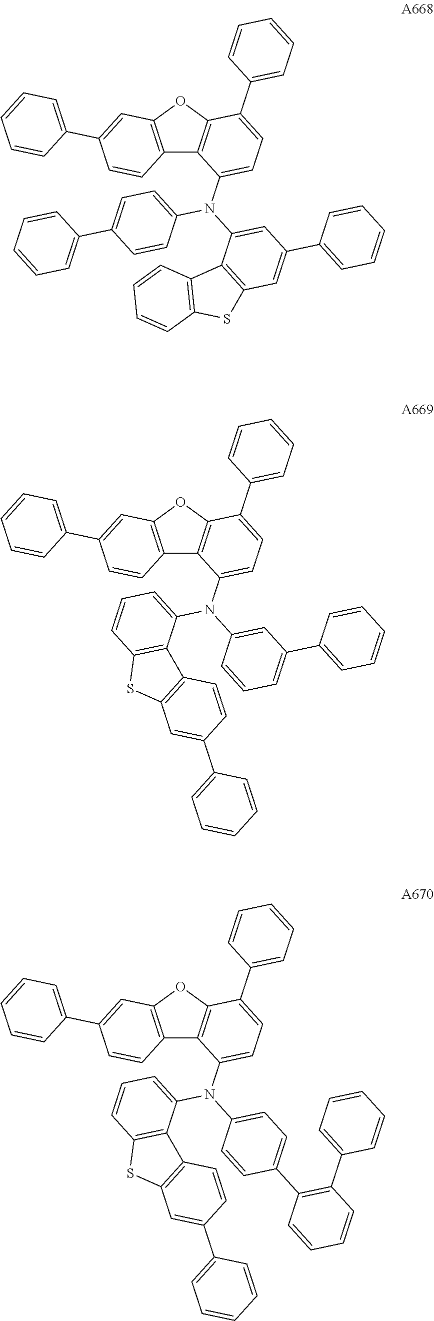 [SMILES: c1ccc(-c2ccc(N(c3cc(-c4ccccc4)cc4sc5ccccc5c34)c3ccc(-c4ccccc4)c4oc5cc(-c6ccccc6)ccc5c34)cc2)cc1 ; c1ccc(-c2cccc(N(c3cccc4sc5cc(-c6ccccc6)ccc5c34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4cccc5sc6cc(-c7ccccc7)ccc6c45)c23)cc1]

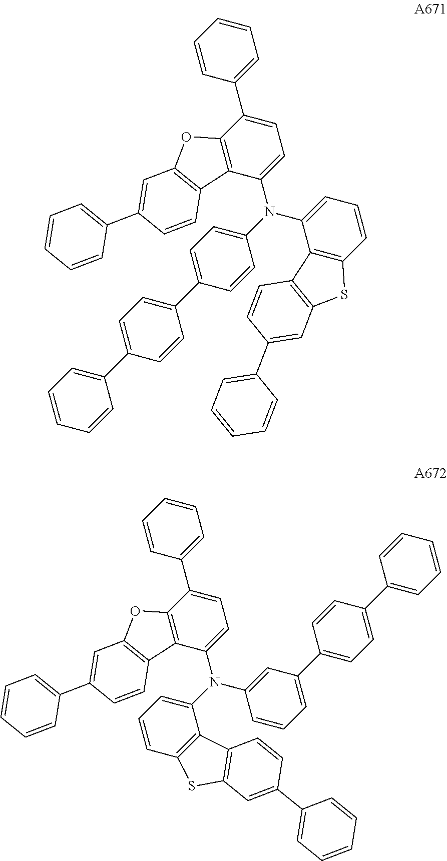 [SMILES: c1ccc(-c2ccc(-c3ccc(N(c4cccc5sc6cc(-c7ccccc7)ccc6c45)c4ccc(-c5ccccc5)c5oc6cc(-c7ccccc7)ccc6c45)cc3)cc2)cc1 ; c1ccc(-c2ccc(-c3cccc(N(c4cccc5sc6cc(-c7ccccc7)ccc6c45)c4ccc(-c5ccccc5)c5oc6cc(-c7ccccc7)ccc6c45)c3)cc2)cc1]

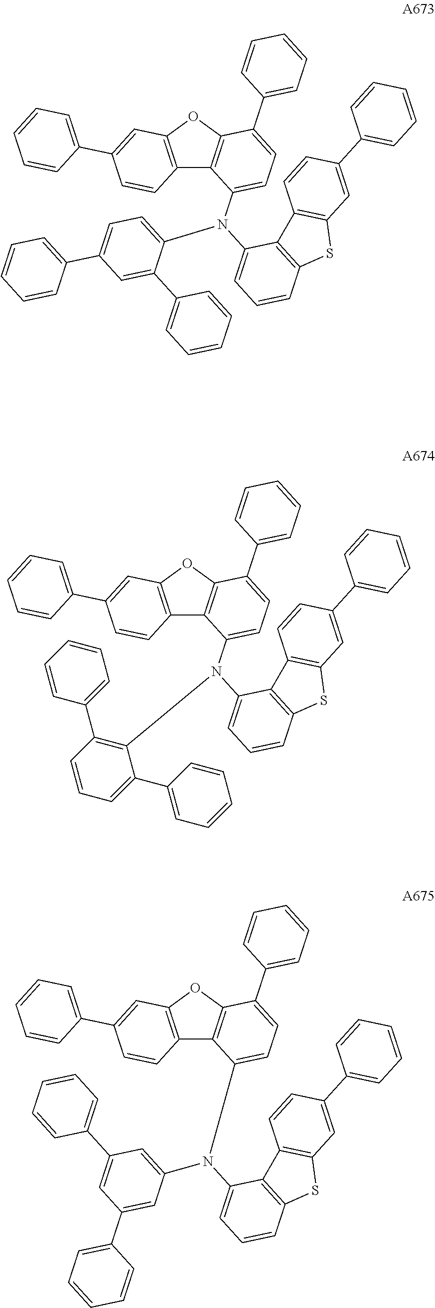 [SMILES: c1ccc(-c2ccc(N(c3cccc4sc5cc(-c6ccccc6)ccc5c34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5sc6cc(-c7ccccc7)ccc6c45)c23)cc1 ; c1ccc(-c2cc(-c3ccccc3)cc(N(c3cccc4sc5cc(-c6ccccc6)ccc5c34)c3ccc(-c4ccccc4)c4oc5cc(-c6ccccc6)ccc5c34)c2)cc1]

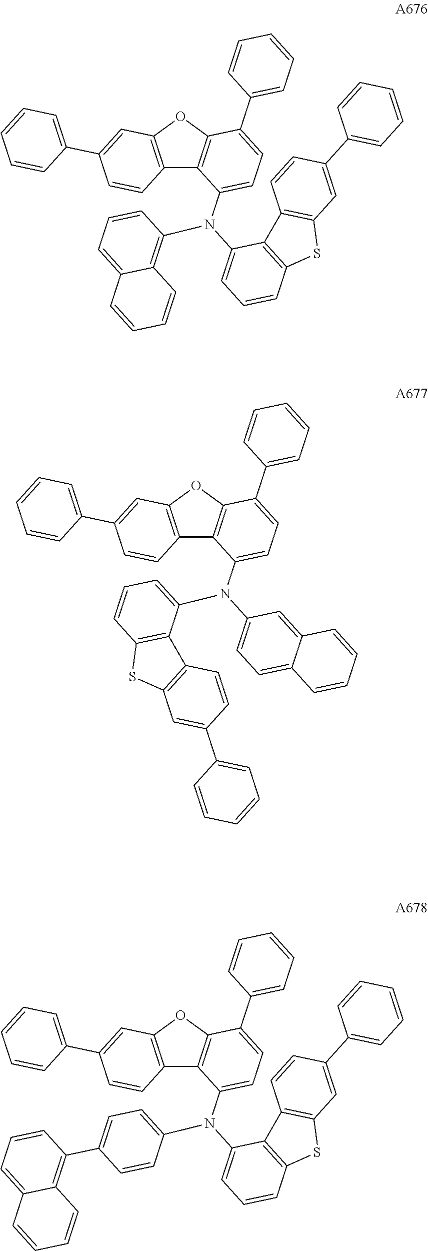 [SMILES: c1ccc(-c2ccc3c(c2)oc2c(-c4ccccc4)ccc(N(c4cccc5ccccc45)c4cccc5sc6cc(-c7ccccc7)ccc6c45)c23)cc1 ; c1ccc(-c2ccc3c(c2)oc2c(-c4ccccc4)ccc(N(c4ccc5ccccc5c4)c4cccc5sc6cc(-c7ccccc7)ccc6c45)c23)cc1 ; c1ccc(-c2ccc3c(c2)oc2c(-c4ccccc4)ccc(N(c4ccc(-c5cccc6ccccc56)cc4)c4cccc5sc6cc(-c7ccccc7)ccc6c45)c23)cc1]

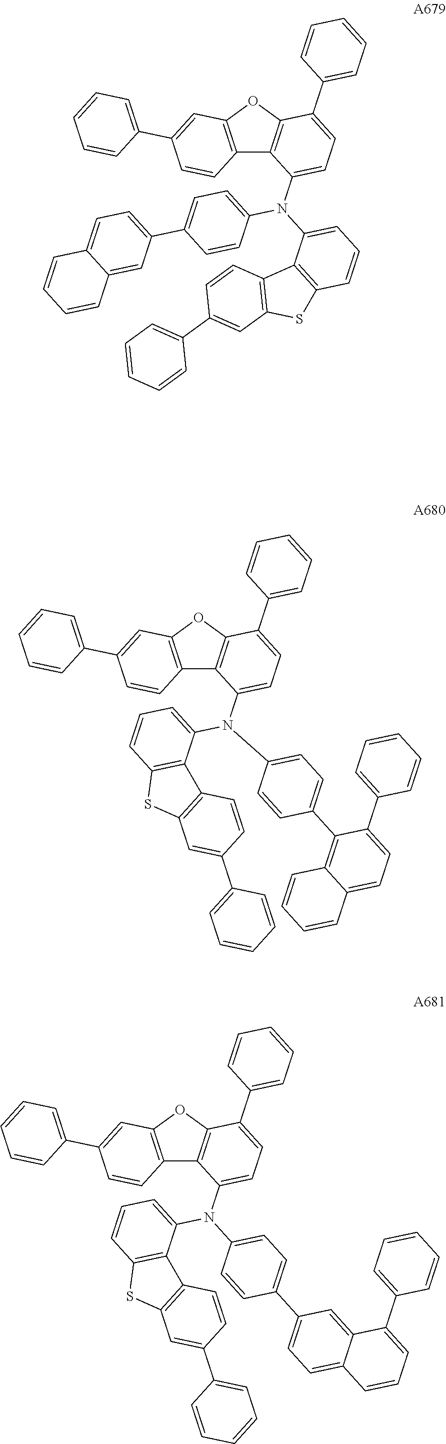 [SMILES: c1ccc(-c2ccc3c(c2)oc2c(-c4ccccc4)ccc(N(c4ccc(-c5ccc6ccccc6c5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5c(-c6ccccc6)ccc6ccccc56)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6cccc(-c7ccccc7)c6c5)cc4)c4cccc5sc6cc(-c7ccccc7)ccc6c45)c23)cc1]

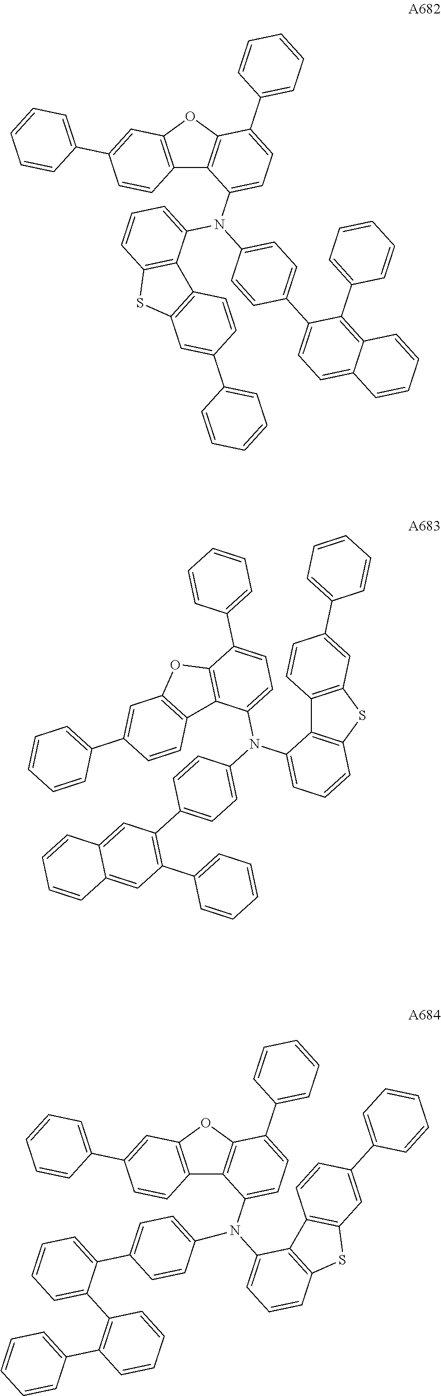 [SMILES: c1ccc(-c2ccc3c(c2)oc2c(-c4ccccc4)ccc(N(c4ccc(-c5ccc6ccccc6c5-c5ccccc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5cc6ccccc6cc5-c5ccccc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccccc5-c5ccccc5-c5ccccc5)cc4)c4cccc5sc6cc(-c7ccccc7)ccc6c45)c23)cc1]

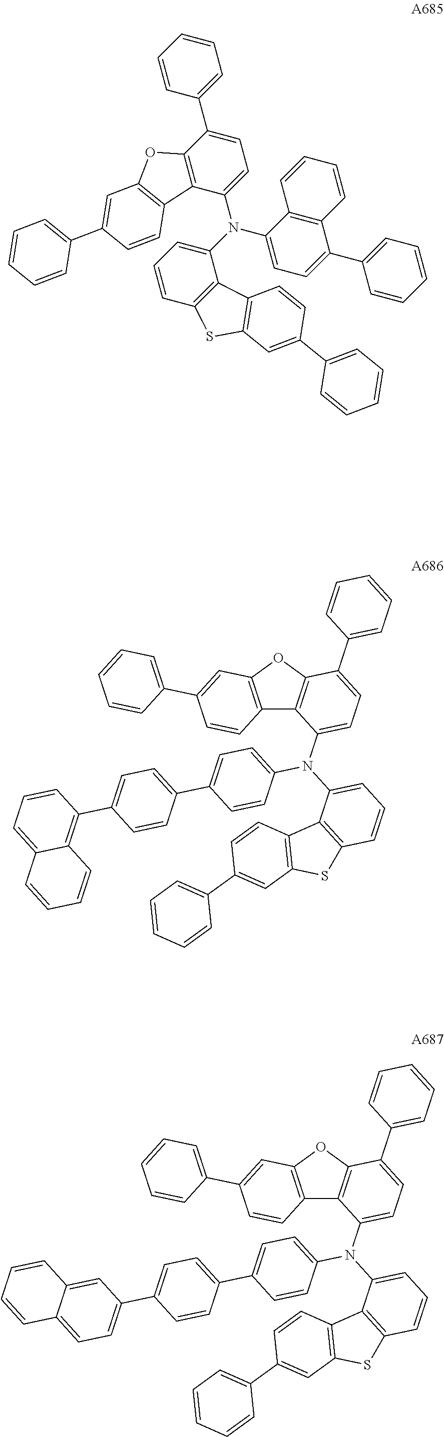 [SMILES: c1ccc(-c2ccc3c(c2)oc2c(-c4ccccc4)ccc(N(c4ccc(-c5ccccc5)c5ccccc45)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6cccc7ccccc67)cc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6ccc7ccccc7c6)cc5)cc4)c4cccc5sc6cc(-c7ccccc7)ccc6c45)c23)cc1]

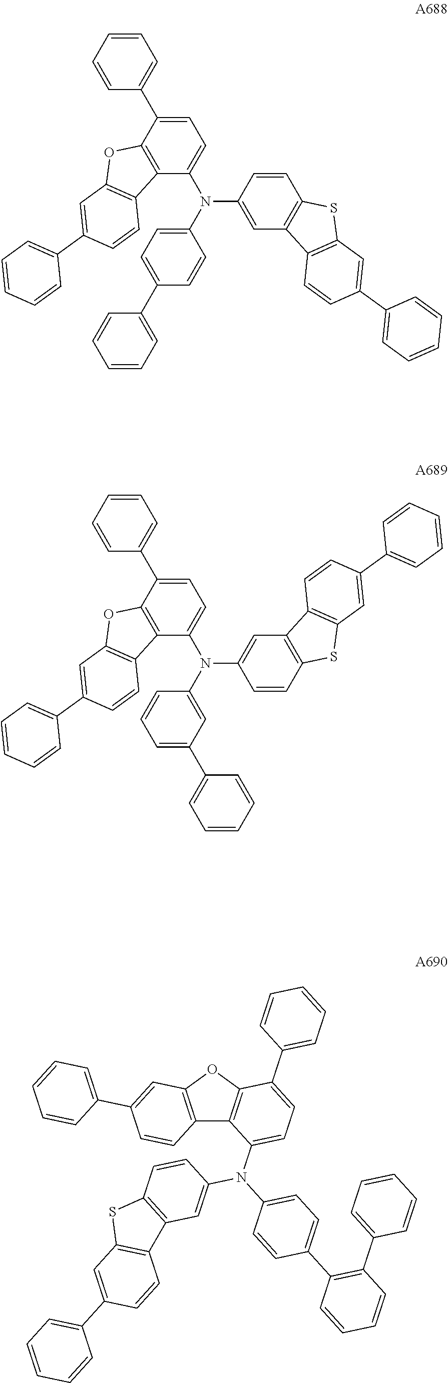 [SMILES: c1ccc(-c2ccc(N(c3ccc4sc5cc(-c6ccccc6)ccc5c4c3)c3ccc(-c4ccccc4)c4oc5cc(-c6ccccc6)ccc5c34)cc2)cc1 ; c1ccc(-c2cccc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4ccc5sc6cc(-c7ccccc7)ccc6c5c4)c23)cc1]

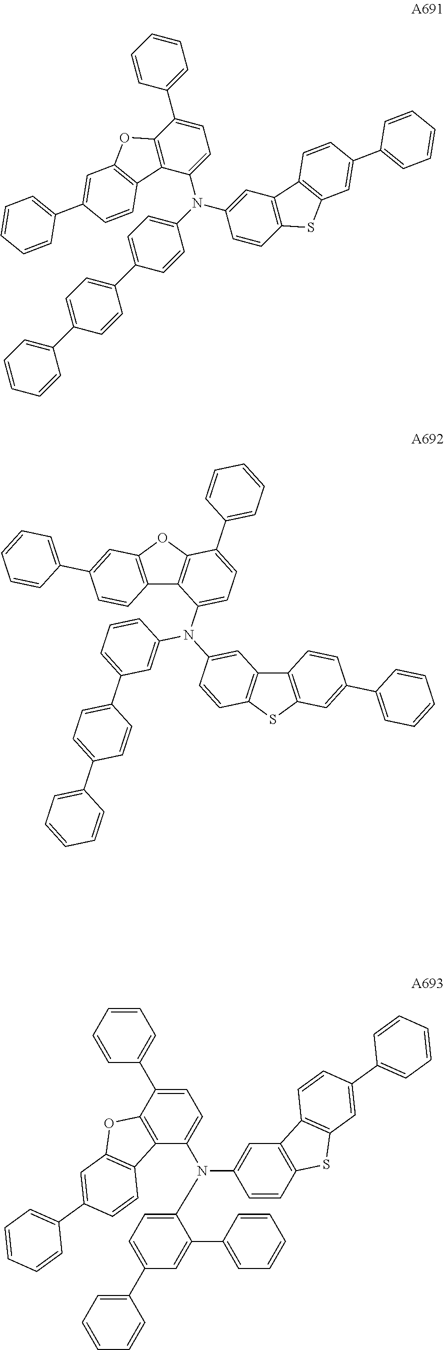 [SMILES: c1ccc(-c2ccc(-c3ccc(N(c4ccc5sc6cc(-c7ccccc7)ccc6c5c4)c4ccc(-c5ccccc5)c5oc6cc(-c7ccccc7)ccc6c45)cc3)cc2)cc1 ; c1ccc(-c2ccc(-c3cccc(N(c4ccc5sc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4sc5cc(-c6ccccc6)ccc5c4c3)c3ccc(-c4ccccc4)c4oc5cc(-c6ccccc6)ccc5c34)c(-c3ccccc3)c2)cc1]

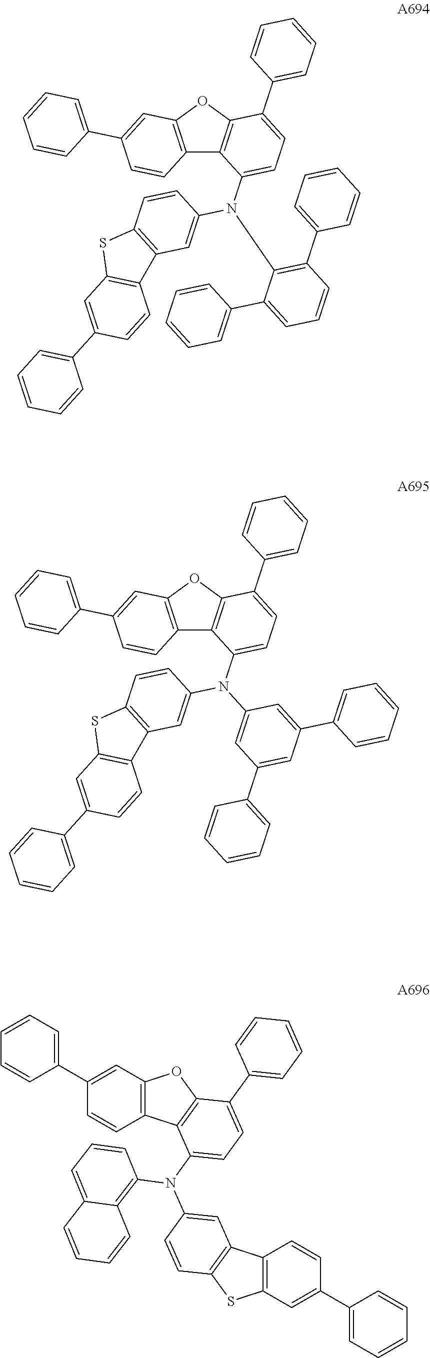 [SMILES: c1ccc(-c2ccc3c(c2)oc2c(-c4ccccc4)ccc(N(c4ccc5sc6cc(-c7ccccc7)ccc6c5c4)c4c(-c5ccccc5)cccc4-c4ccccc4)c23)cc1 ; c1ccc(-c2cc(-c3ccccc3)cc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5sc6cc(-c7ccccc7)ccc6c5c4)c4cccc5ccccc45)c23)cc1]

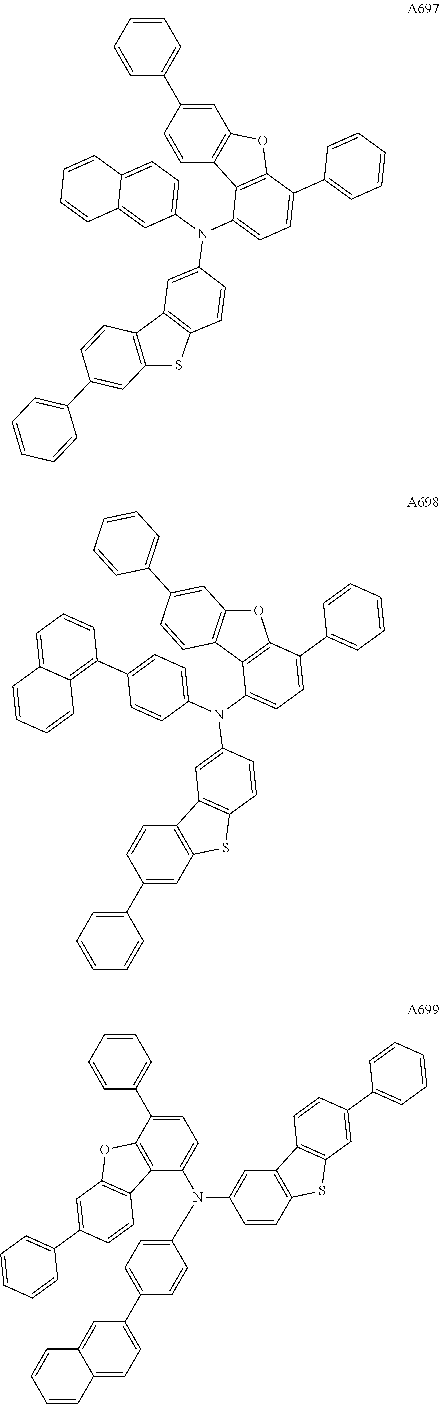 [SMILES: c1ccc(-c2ccc3c(c2)oc2c(-c4ccccc4)ccc(N(c4ccc5ccccc5c4)c4ccc5sc6cc(-c7ccccc7)ccc6c5c4)c23)cc1 ; c1ccc(-c2ccc3c(c2)oc2c(-c4ccccc4)ccc(N(c4ccc(-c5cccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5)cc4)c4ccc5sc6cc(-c7ccccc7)ccc6c5c4)c23)cc1]

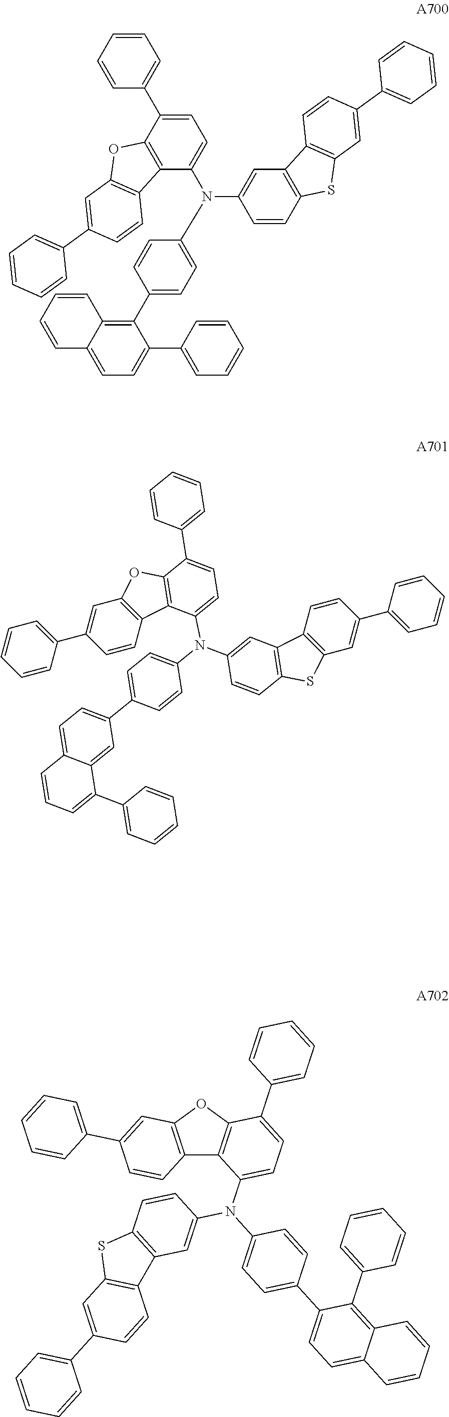 [SMILES: c1ccc(-c2ccc3c(c2)oc2c(-c4ccccc4)ccc(N(c4ccc(-c5c(-c6ccccc6)ccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6cccc(-c7ccccc7)c6c5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5-c5ccccc5)cc4)c4ccc5sc6cc(-c7ccccc7)ccc6c5c4)c23)cc1]

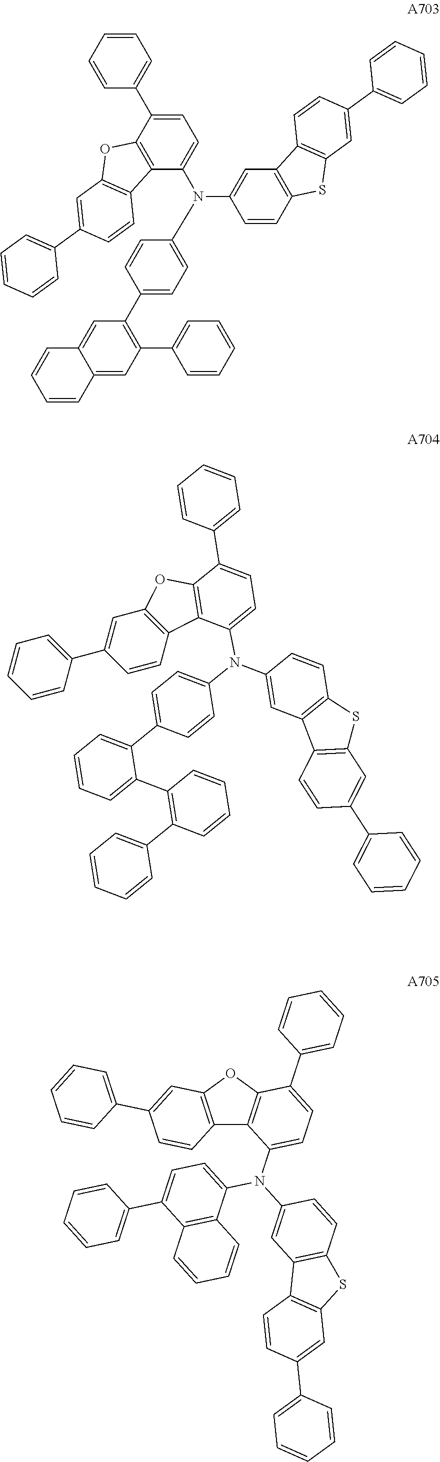 [SMILES: c1ccc(-c2ccc3c(c2)oc2c(-c4ccccc4)ccc(N(c4ccc(-c5cc6ccccc6cc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccccc5-c5ccccc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc5sc6cc(-c7ccccc7)ccc6c5c4)c4ccc(-c5ccccc5)c5ccccc45)c23)cc1]

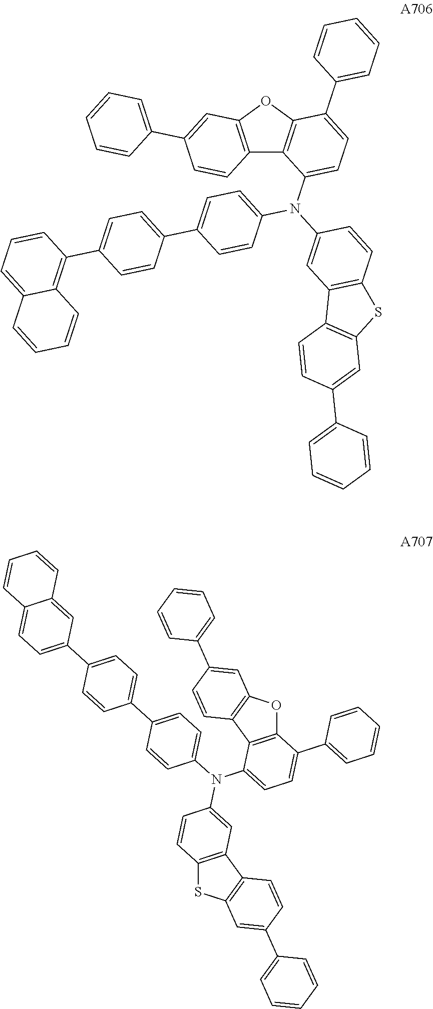 [SMILES: c1ccc(-c2ccc3c(c2)oc2c(-c4ccccc4)ccc(N(c4ccc(-c5ccc(-c6cccc7ccccc67)cc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5sc6cc(-c7ccccc7)ccc6c5c4)c23)cc1]

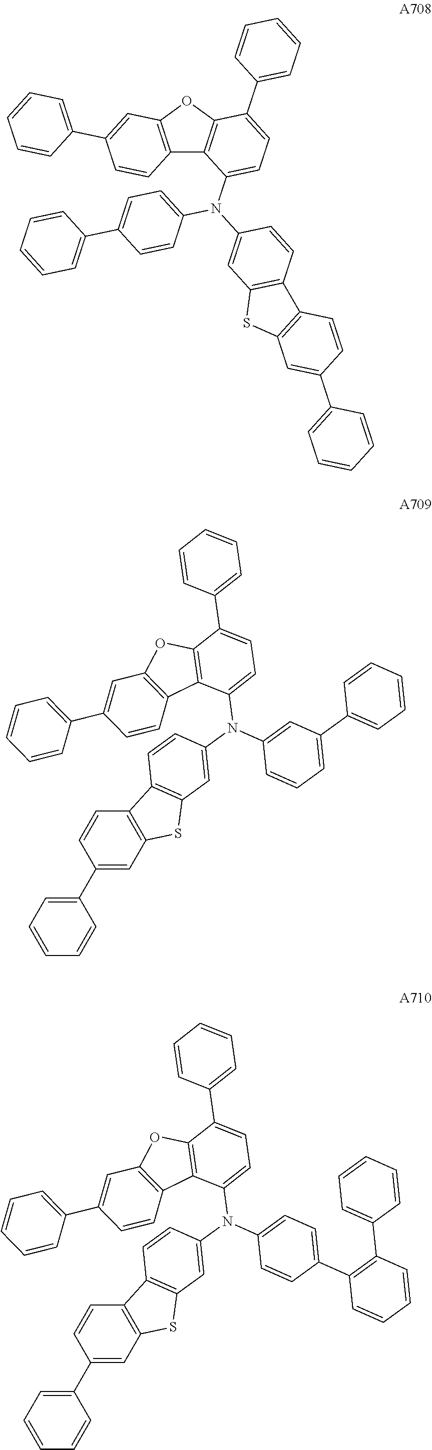 [SMILES: c1ccc(-c2ccc(N(c3ccc4c(c3)sc3cc(-c5ccccc5)ccc34)c3ccc(-c4ccccc4)c4oc5cc(-c6ccccc6)ccc5c34)cc2)cc1 ; c1ccc(-c2cccc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4ccc5c(c4)sc4cc(-c6ccccc6)ccc45)c23)cc1]

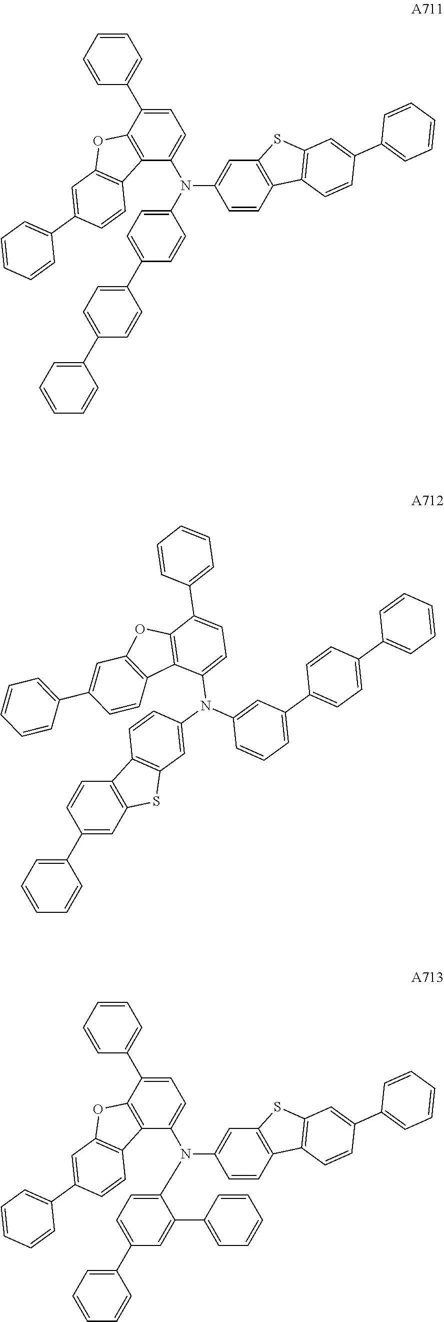 [SMILES: c1ccc(-c2ccc(-c3ccc(N(c4ccc5c(c4)sc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)sc4cc(-c6ccccc6)ccc45)c4ccc(-c5ccccc5)c5oc6cc(-c7ccccc7)ccc6c45)c3)cc2)cc1 ; c1ccc(-c2ccc(N(c3ccc4c(c3)sc3cc(-c5ccccc5)ccc34)c3ccc(-c4ccccc4)c4oc5cc(-c6ccccc6)ccc5c34)c(-c3ccccc3)c2)cc1]

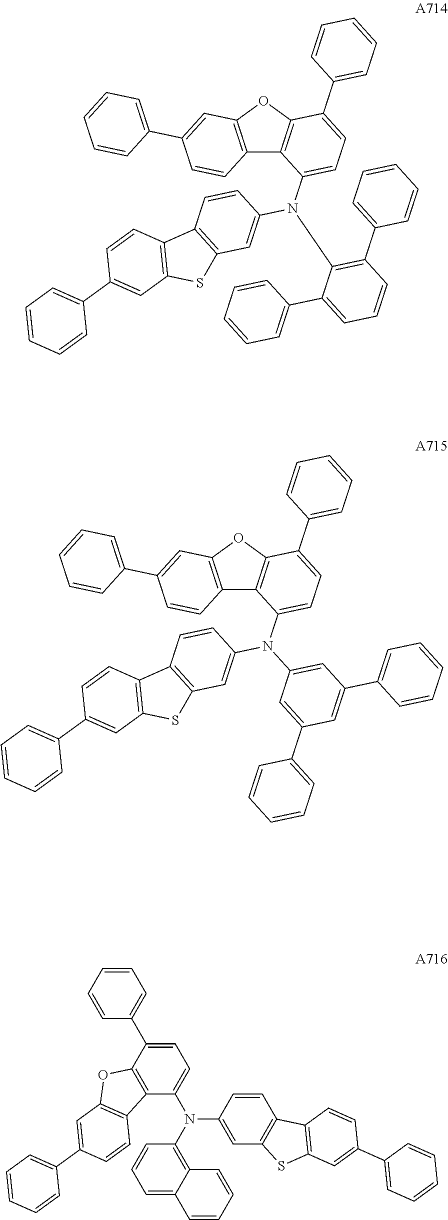 [SMILES: c1ccc(-c2ccc3c(c2)oc2c(-c4ccccc4)ccc(N(c4ccc5c(c4)sc4cc(-c6ccccc6)ccc45)c4c(-c5ccccc5)cccc4-c4ccccc4)c23)cc1 ; c1ccc(-c2cc(-c3ccccc3)cc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5c(c4)sc4cc(-c6ccccc6)ccc45)c4cccc5ccccc45)c23)cc1]

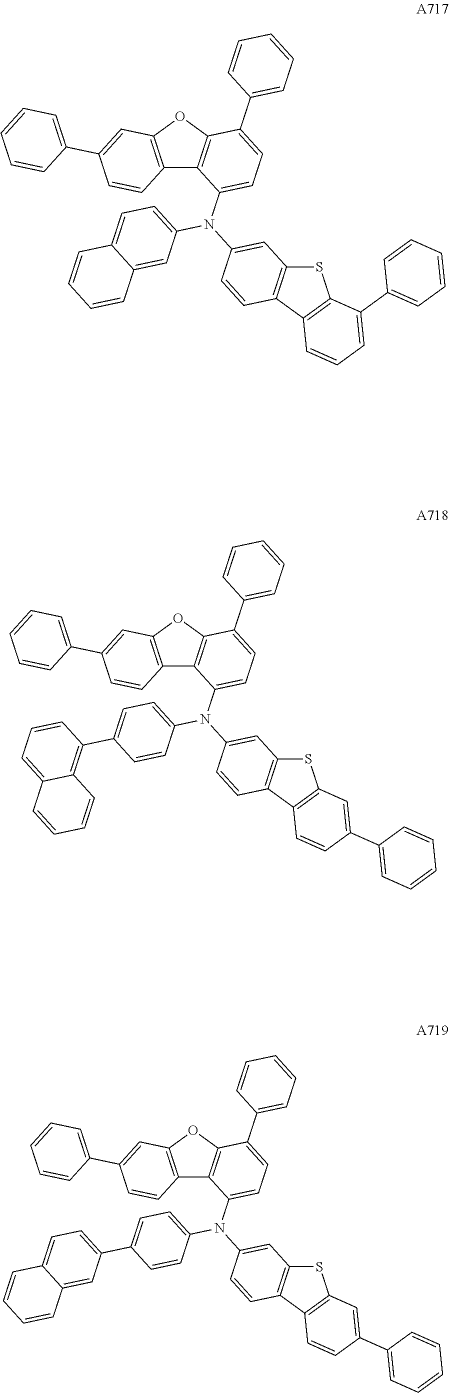 [SMILES: c1ccc(-c2ccc3c(c2)oc2c(-c4ccccc4)ccc(N(c4ccc5ccccc5c4)c4ccc5c(c4)sc4c(-c6ccccc6)cccc45)c23)cc1 ; c1ccc(-c2ccc3c(c2)oc2c(-c4ccccc4)ccc(N(c4ccc(-c5cccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5)cc4)c4ccc5c(c4)sc4cc(-c6ccccc6)ccc45)c23)cc1]

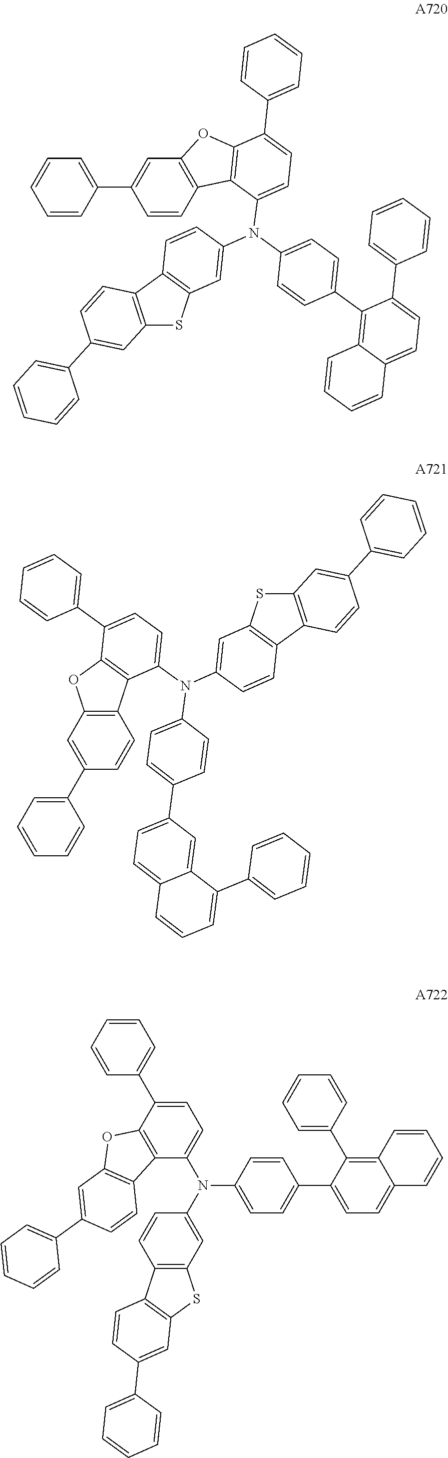 [SMILES: c1ccc(-c2ccc3c(c2)oc2c(-c4ccccc4)ccc(N(c4ccc(-c5c(-c6ccccc6)ccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6cccc(-c7ccccc7)c6c5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5-c5ccccc5)cc4)c4ccc5c(c4)sc4cc(-c6ccccc6)ccc45)c23)cc1]

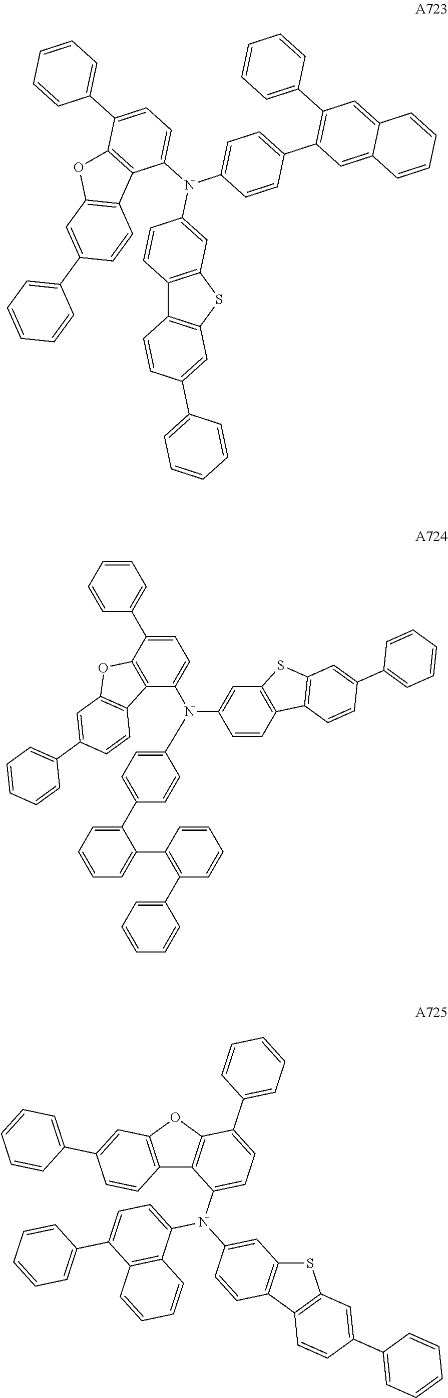 [SMILES: c1ccc(-c2ccc3c(c2)oc2c(-c4ccccc4)ccc(N(c4ccc(-c5cc6ccccc6cc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccccc5-c5ccccc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc5c(c4)sc4cc(-c6ccccc6)ccc45)c4ccc(-c5ccccc5)c5ccccc45)c23)cc1]

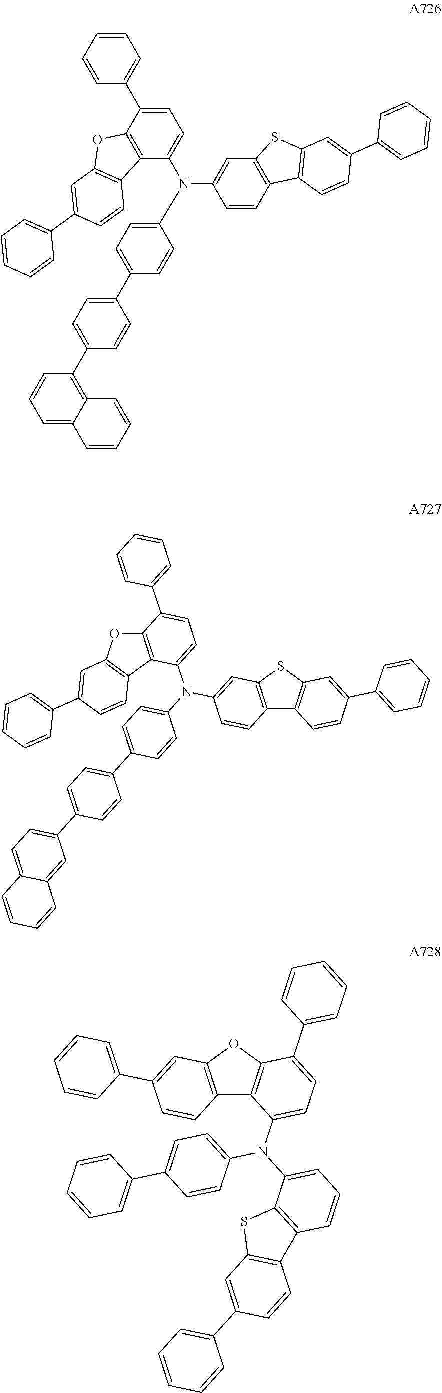 [SMILES: c1ccc(-c2ccc3c(c2)oc2c(-c4ccccc4)ccc(N(c4ccc(-c5ccc(-c6cccc7ccccc67)cc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5c(c4)sc4cc(-c6ccccc6)ccc45)c23)cc1 ; c1ccc(-c2ccc(N(c3cccc4c3sc3cc(-c5ccccc5)ccc34)c3ccc(-c4ccccc4)c4oc5cc(-c6ccccc6)ccc5c34)cc2)cc1]

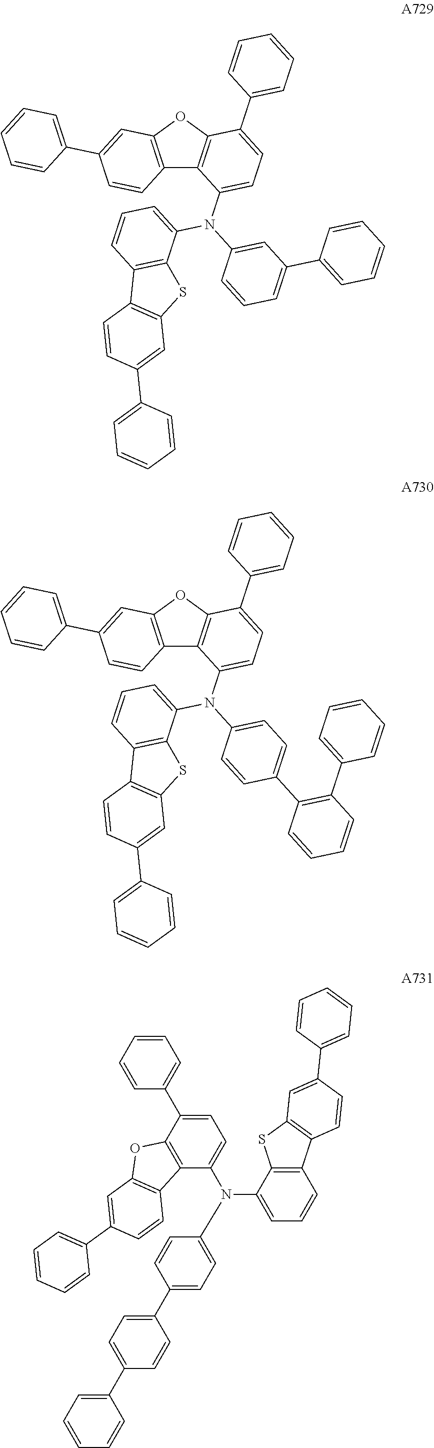 [SMILES: c1ccc(-c2cccc(N(c3cccc4c3sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4cccc5c4sc4cc(-c6ccccc6)ccc45)c23)cc1 ; c1ccc(-c2ccc(-c3ccc(N(c4cccc5c4sc4cc(-c6ccccc6)ccc45)c4ccc(-c5ccccc5)c5oc6cc(-c7ccccc7)ccc6c45)cc3)cc2)cc1]

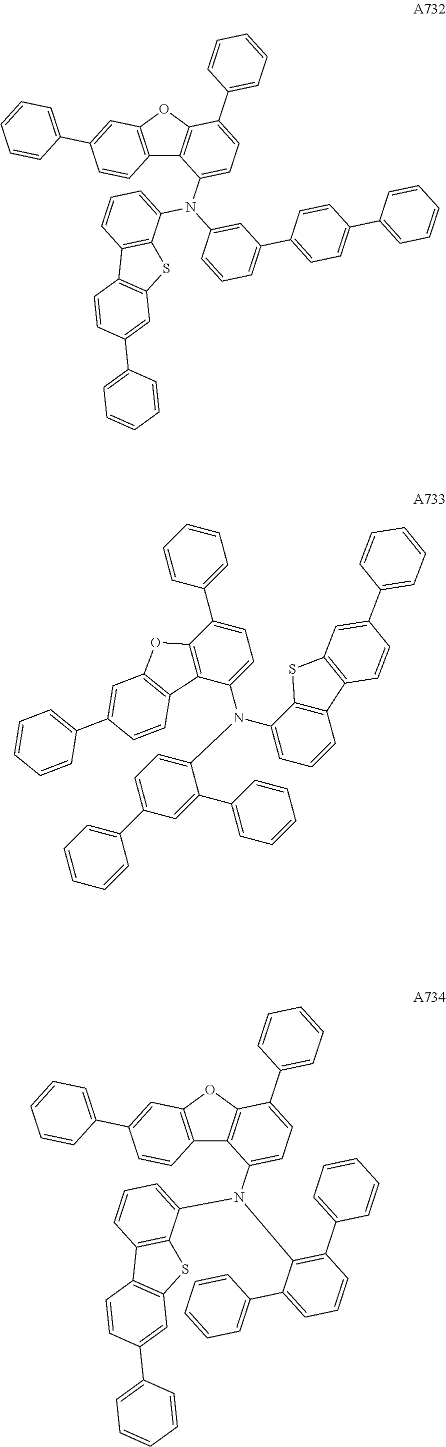 [SMILES: c1ccc(-c2ccc(-c3cccc(N(c4cccc5c4sc4cc(-c6ccccc6)ccc45)c4ccc(-c5ccccc5)c5oc6cc(-c7ccccc7)ccc6c45)c3)cc2)cc1 ; c1ccc(-c2ccc(N(c3cccc4c3sc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5c4sc4cc(-c6ccccc6)ccc45)c23)cc1]

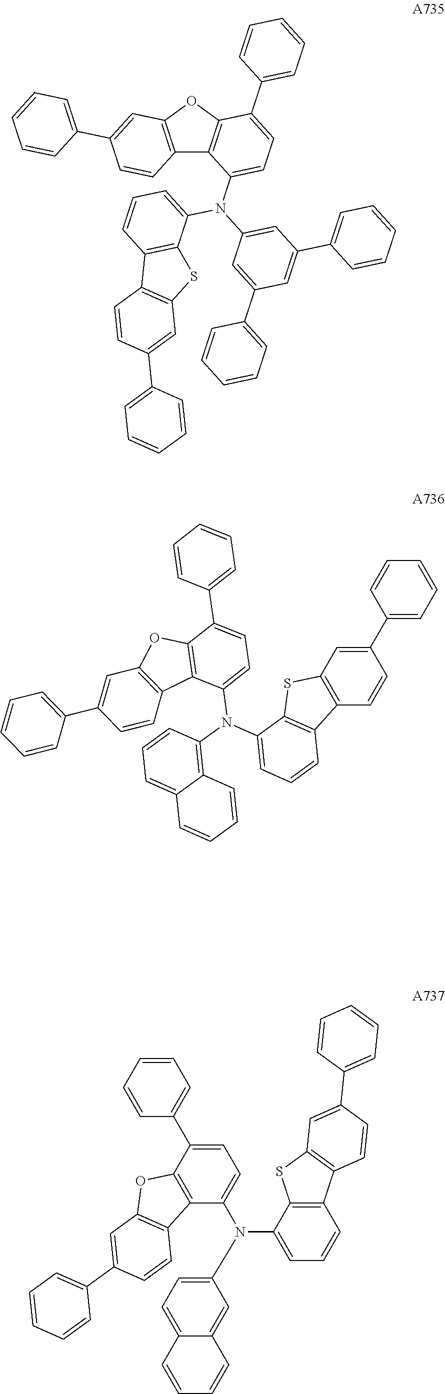 [SMILES: c1ccc(-c2cc(-c3ccccc3)cc(N(c3cccc4c3sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4cccc5ccccc45)c4cccc5c4sc4cc(-c6ccccc6)ccc45)c23)cc1 ; c1ccc(-c2ccc3c(c2)oc2c(-c4ccccc4)ccc(N(c4ccc5ccccc5c4)c4cccc5c4sc4cc(-c6ccccc6)ccc45)c23)cc1]

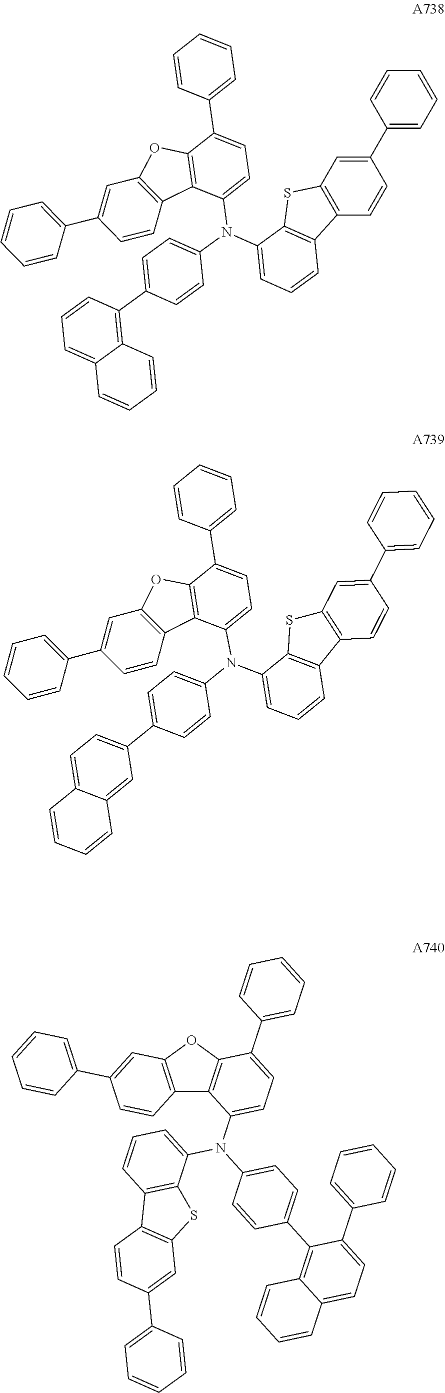 [SMILES: c1ccc(-c2ccc3c(c2)oc2c(-c4ccccc4)ccc(N(c4ccc(-c5cccc6ccccc56)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6ccccc6c5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5c(-c6ccccc6)ccc6ccccc56)cc4)c4cccc5c4sc4cc(-c6ccccc6)ccc45)c23)cc1]

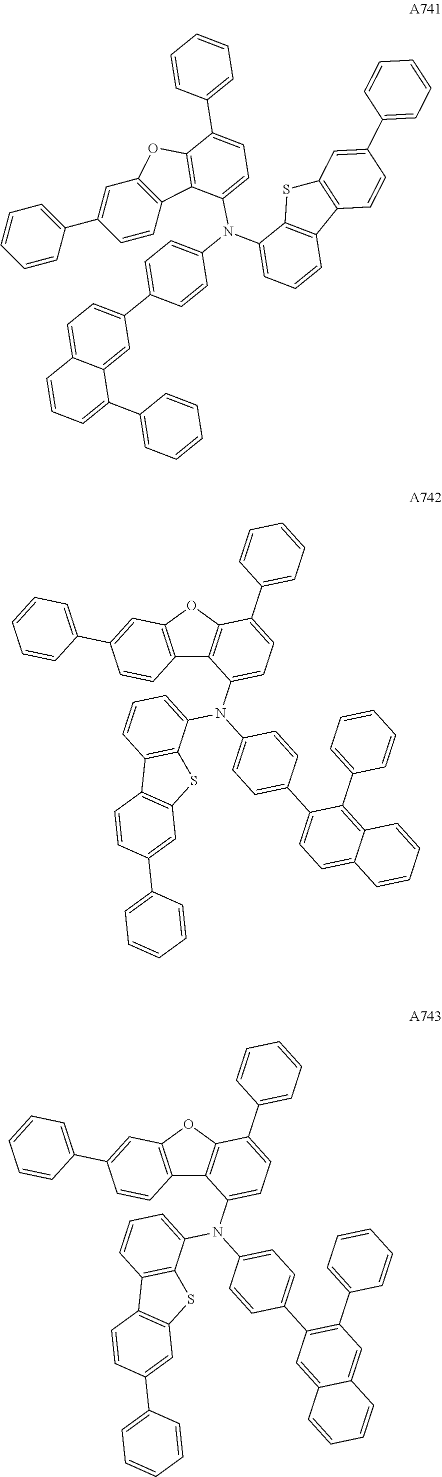 [SMILES: c1ccc(-c2ccc3c(c2)oc2c(-c4ccccc4)ccc(N(c4ccc(-c5ccc6cccc(-c7ccccc7)c6c5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6ccccc6c5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5cc6ccccc6cc5-c5ccccc5)cc4)c4cccc5c4sc4cc(-c6ccccc6)ccc45)c23)cc1]

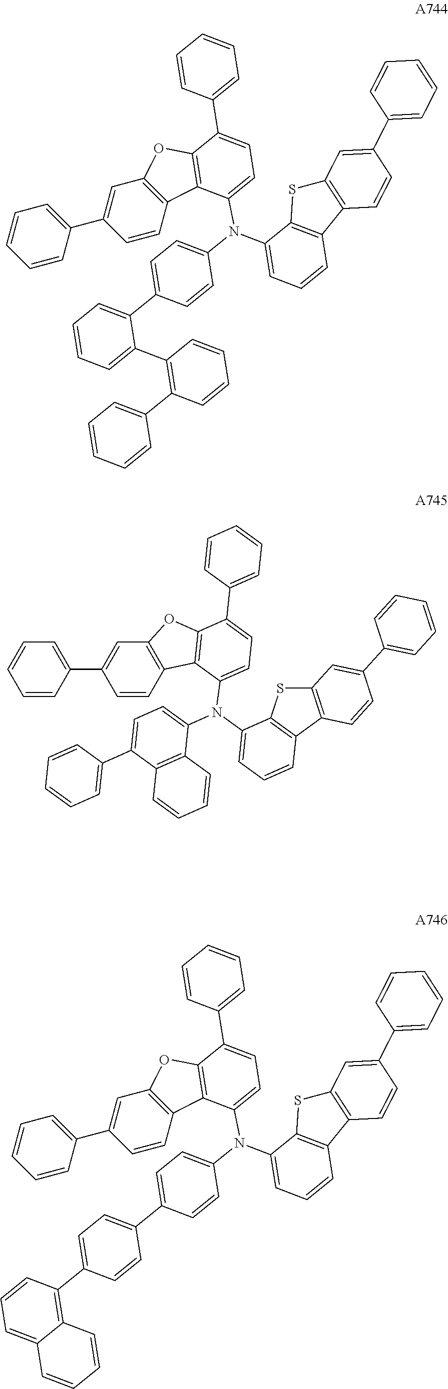 [SMILES: c1ccc(-c2ccc3c(c2)oc2c(-c4ccccc4)ccc(N(c4ccc(-c5ccccc5-c5ccccc5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccccc5)c5ccccc45)c4cccc5c4sc4cc(-c6ccccc6)ccc45)c23)cc1 ; c1ccc(-c2ccc3c(c2)oc2c(-c4ccccc4)ccc(N(c4ccc(-c5ccc(-c6cccc7ccccc67)cc5)cc4)c4cccc5c4sc4cc(-c6ccccc6)ccc45)c23)cc1]

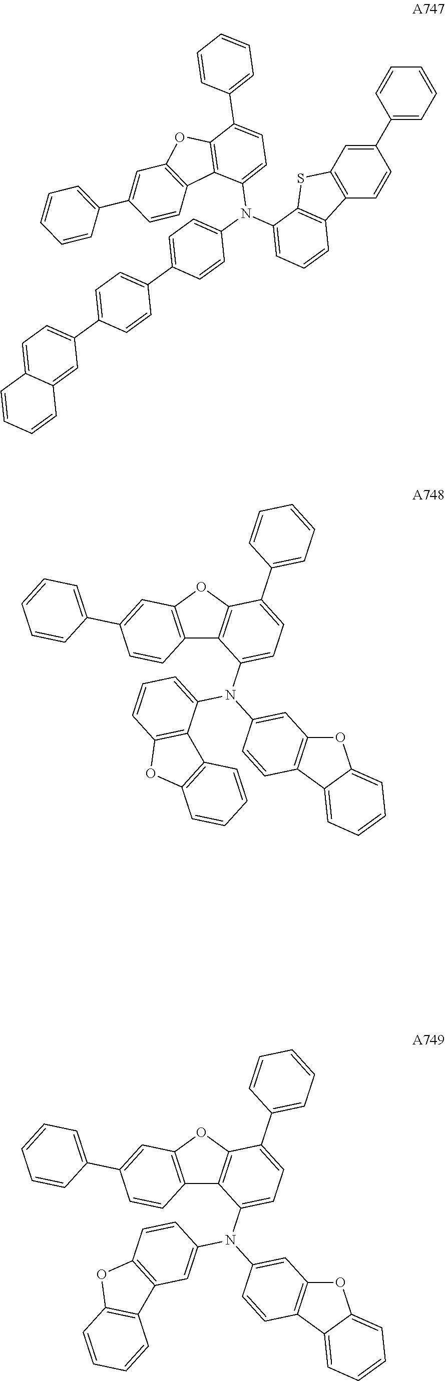 [SMILES: c1ccc(-c2ccc3c(c2)oc2c(-c4ccccc4)ccc(N(c4ccc(-c5ccc(-c6ccc7ccccc7c6)cc5)cc4)c4cccc5c4sc4cc(-c6ccccc6)ccc45)c23)cc1 ; c1ccc(-c2ccc3c(c2)oc2c(-c4ccccc4)ccc(N(c4ccc5c(c4)oc4ccccc45)c4cccc5oc6ccccc6c45)c23)cc1 ; c1ccc(-c2ccc3c(c2)oc2c(-c4ccccc4)ccc(N(c4ccc5c(c4)oc4ccccc45)c4ccc5oc6ccccc6c5c4)c23)cc1]

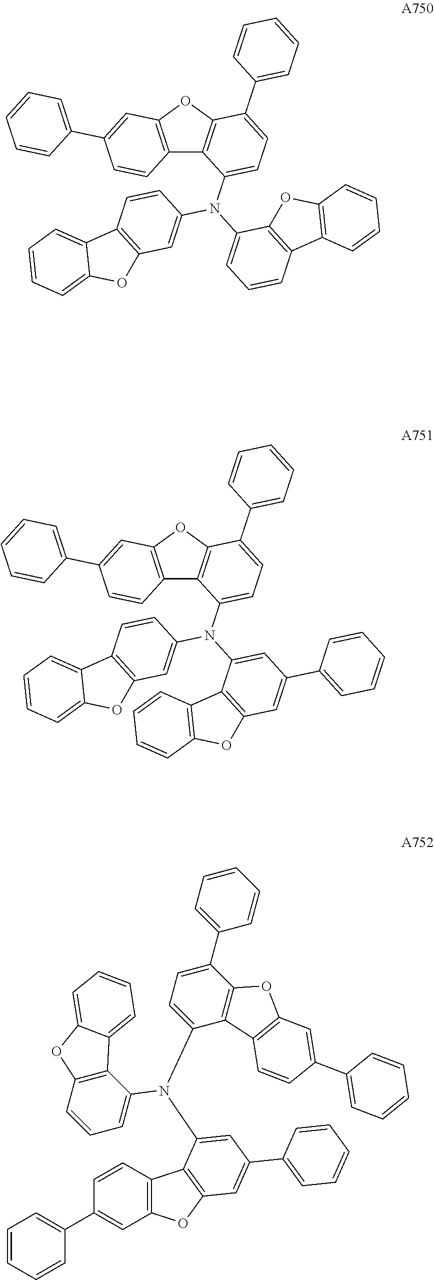 [SMILES: c1ccc(-c2ccc3c(c2)oc2c(-c4ccccc4)ccc(N(c4ccc5c(c4)oc4ccccc45)c4cccc5c4oc4ccccc45)c23)cc1 ; c1ccc(-c2ccc3c(c2)oc2c(-c4ccccc4)ccc(N(c4ccc5c(c4)oc4ccccc45)c4cc(-c5ccccc5)cc5oc6ccccc6c45)c23)cc1 ; c1ccc(-c2ccc3c(c2)oc2cc(-c4ccccc4)cc(N(c4cccc5oc6ccccc6c45)c4ccc(-c5ccccc5)c5oc6cc(-c7ccccc7)ccc6c45)c23)cc1]

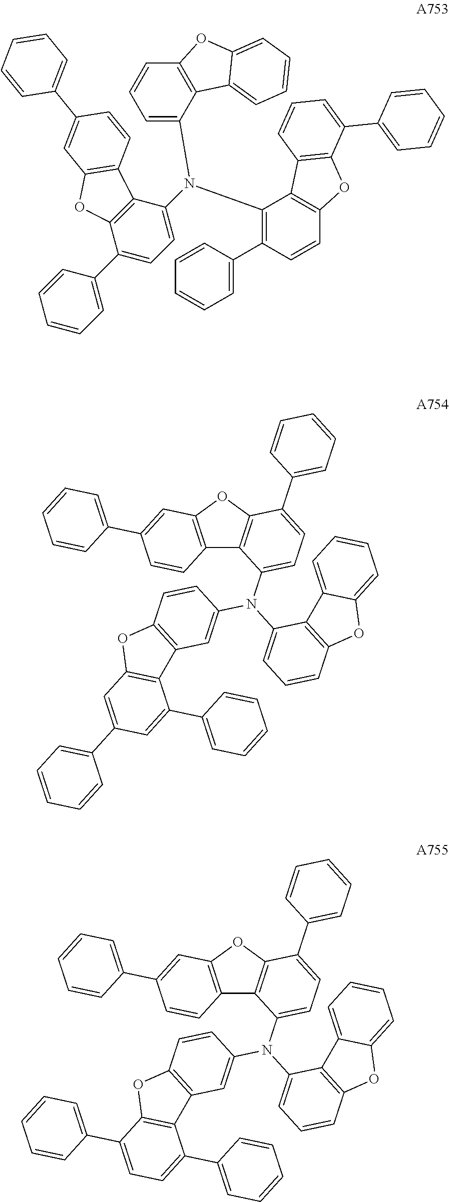 [SMILES: c1ccc(-c2ccc3c(c2)oc2c(-c4ccccc4)ccc(N(c4cccc5oc6ccccc6c45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5oc6ccccc6c45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5oc6ccccc6c45)c23)cc1]

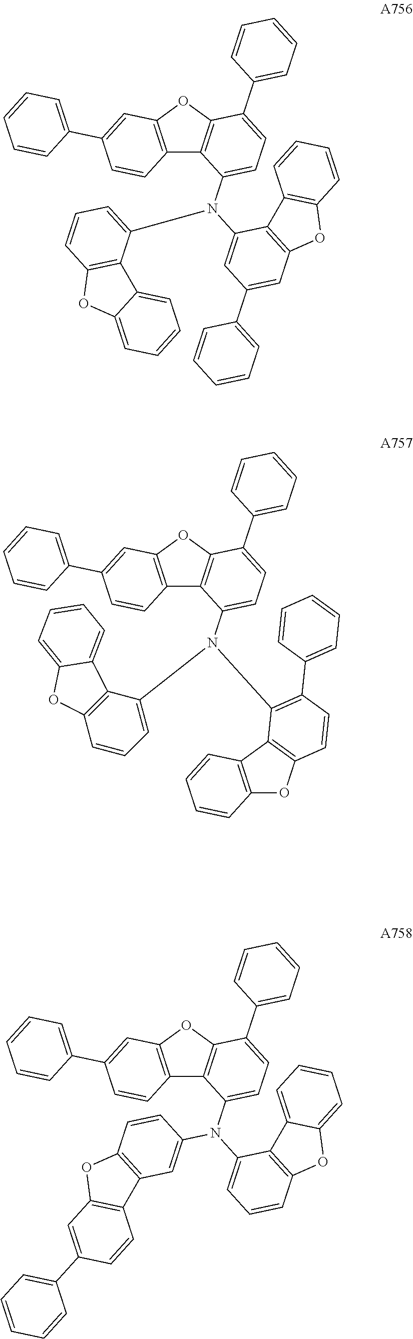 [SMILES: c1ccc(-c2ccc3c(c2)oc2c(-c4ccccc4)ccc(N(c4cccc5oc6ccccc6c45)c4cc(-c5ccccc5)cc5oc6ccccc6c45)c23)cc1 ; c1ccc(-c2ccc3c(c2)oc2c(-c4ccccc4)ccc(N(c4cccc5oc6ccccc6c45)c4c(-c5ccccc5)ccc5oc6ccccc6c45)c23)cc1 ; c1ccc(-c2ccc3c(c2)oc2ccc(N(c4cccc5oc6ccccc6c45)c4ccc(-c5ccccc5)c5oc6cc(-c7ccccc7)ccc6c45)cc23)cc1]

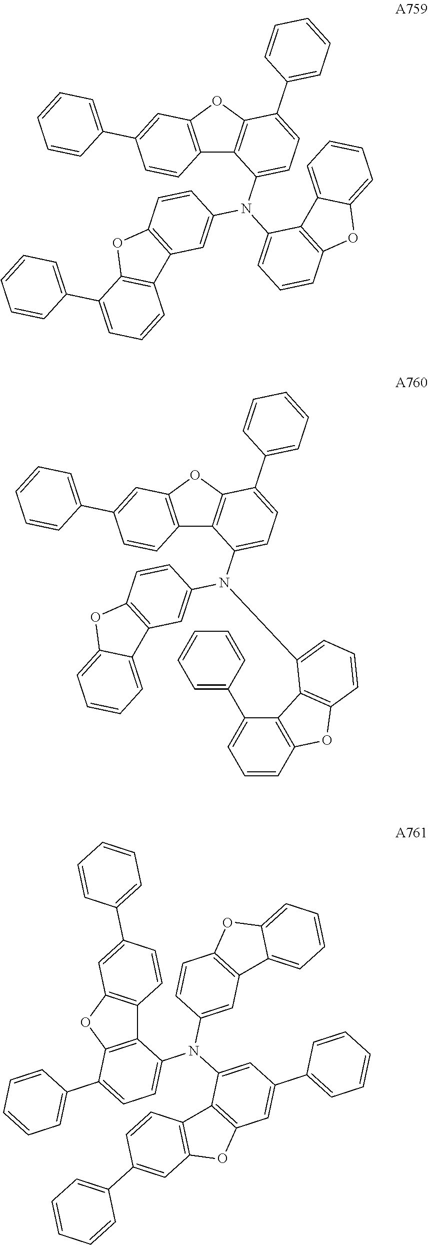 [SMILES: c1ccc(-c2ccc3c(c2)oc2c(-c4ccccc4)ccc(N(c4ccc5oc6c(-c7ccccc7)cccc6c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5oc6ccccc6c5c4)c4ccc(-c5ccccc5)c5oc6cc(-c7ccccc7)ccc6c45)c23)cc1]

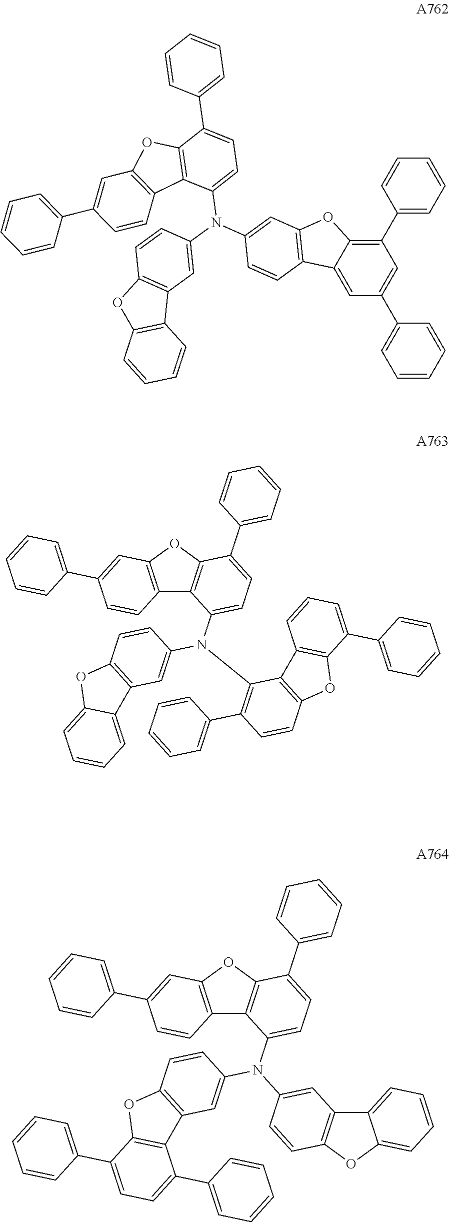 [SMILES: c1ccc(-c2ccc3c(c2)oc2c(-c4ccccc4)ccc(N(c4ccc5c(c4)oc4c(-c6ccccc6)cc(-c6ccccc6)cc45)c4ccc5oc6ccccc6c5c4)c23)cc1 ; c1ccc(-c2ccc3c(c2)oc2c(-c4ccccc4)ccc(N(c4ccc5oc6ccccc6c5c4)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6ccccc6c5c4)c4ccc5oc6c(-c7ccccc7)ccc(-c7ccccc7)c6c5c4)c23)cc1]

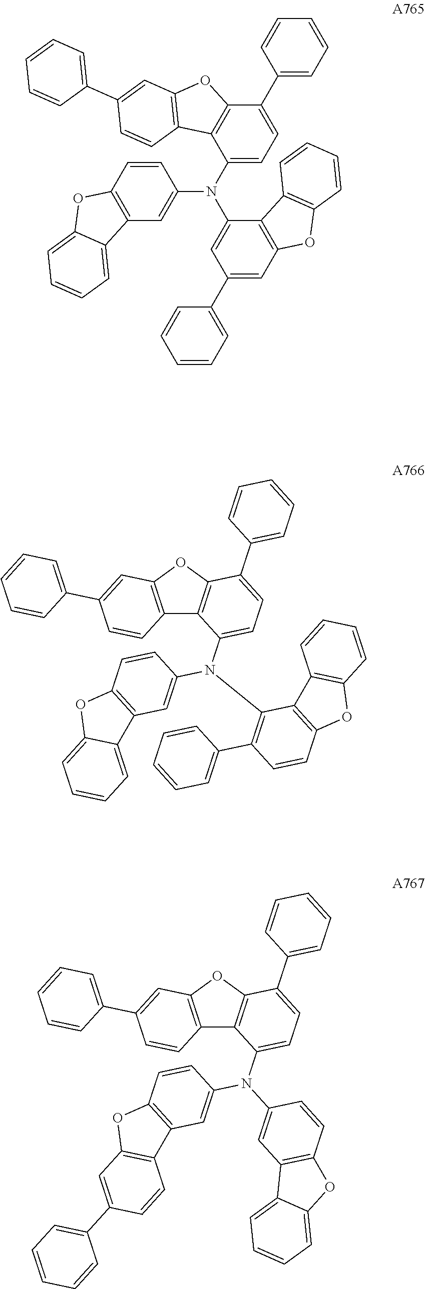 [SMILES: c1ccc(-c2ccc3c(c2)oc2c(-c4ccccc4)ccc(N(c4ccc5oc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc5oc6ccccc6c5c4)c4c(-c5ccccc5)ccc5oc6ccccc6c45)c23)cc1 ; c1ccc(-c2ccc3c(c2)oc2ccc(N(c4ccc5oc6ccccc6c5c4)c4ccc(-c5ccccc5)c5oc6cc(-c7ccccc7)ccc6c45)cc23)cc1]

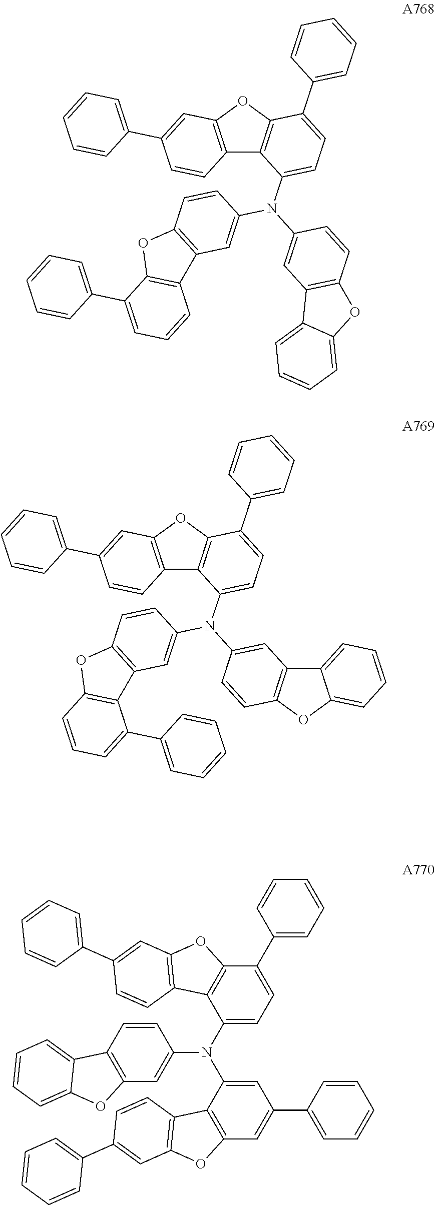 [SMILES: c1ccc(-c2ccc3c(c2)oc2c(-c4ccccc4)ccc(N(c4ccc5oc6ccccc6c5c4)c4ccc5oc6c(-c7ccccc7)cccc6c5c4)c23)cc1 ; c1ccc(-c2ccc3c(c2)oc2c(-c4ccccc4)ccc(N(c4ccc5oc6ccccc6c5c4)c4ccc5oc6cccc(-c7ccccc7)c6c5c4)c23)cc1 ; c1ccc(-c2ccc3c(c2)oc2cc(-c4ccccc4)cc(N(c4ccc5c(c4)oc4ccccc45)c4ccc(-c5ccccc5)c5oc6cc(-c7ccccc7)ccc6c45)c23)cc1]

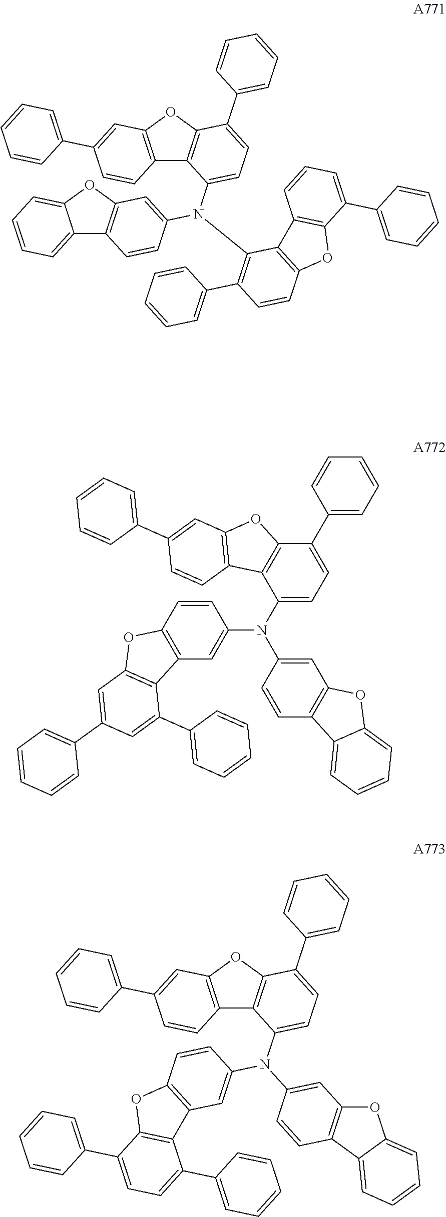 [SMILES: c1ccc(-c2ccc3c(c2)oc2c(-c4ccccc4)ccc(N(c4ccc5c(c4)oc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5c(c4)oc4ccccc45)c4ccc5oc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5oc6c(-c7ccccc7)ccc(-c7ccccc7)c6c5c4)c23)cc1]

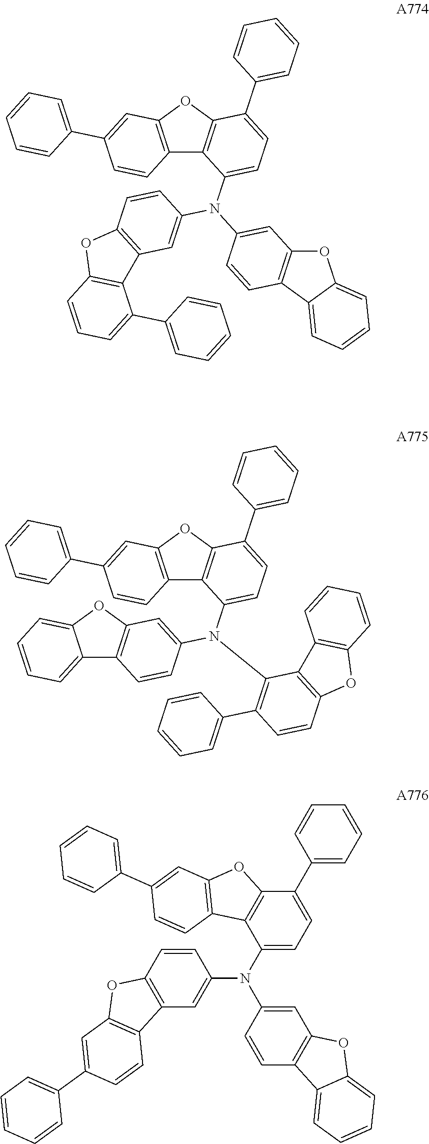 [SMILES: c1ccc(-c2ccc3c(c2)oc2c(-c4ccccc4)ccc(N(c4ccc5c(c4)oc4ccccc45)c4ccc5oc6cccc(-c7ccccc7)c6c5c4)c23)cc1 ; c1ccc(-c2ccc3c(c2)oc2c(-c4ccccc4)ccc(N(c4ccc5c(c4)oc4ccccc45)c4c(-c5ccccc5)ccc5oc6ccccc6c45)c23)cc1 ; c1ccc(-c2ccc3c(c2)oc2ccc(N(c4ccc5c(c4)oc4ccccc45)c4ccc(-c5ccccc5)c5oc6cc(-c7ccccc7)ccc6c45)cc23)cc1]

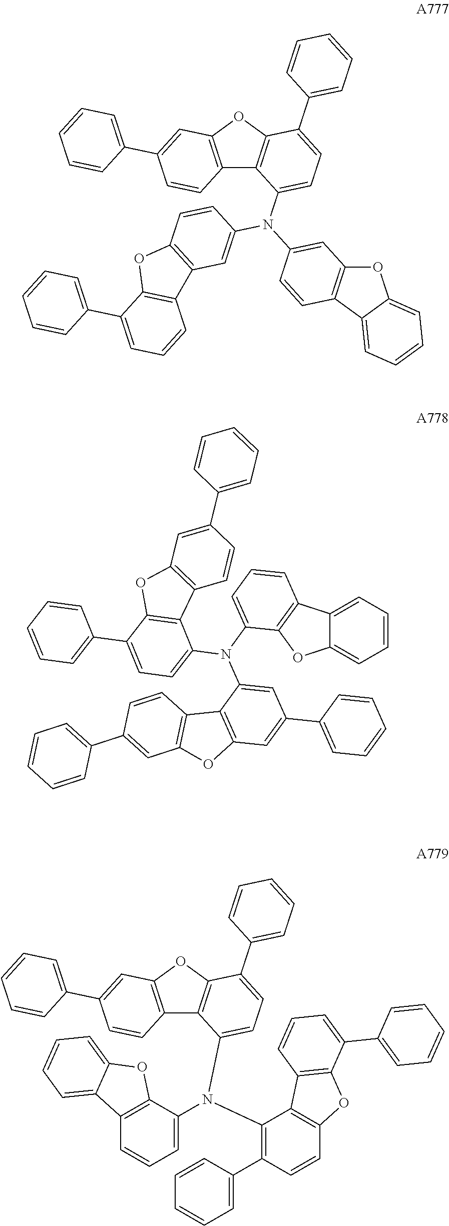 [SMILES: c1ccc(-c2ccc3c(c2)oc2c(-c4ccccc4)ccc(N(c4ccc5c(c4)oc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4oc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4cccc5c4oc4ccccc45)c4c(-c5ccccc5)ccc5oc6c(-c7ccccc7)cccc6c45)c23)cc1]

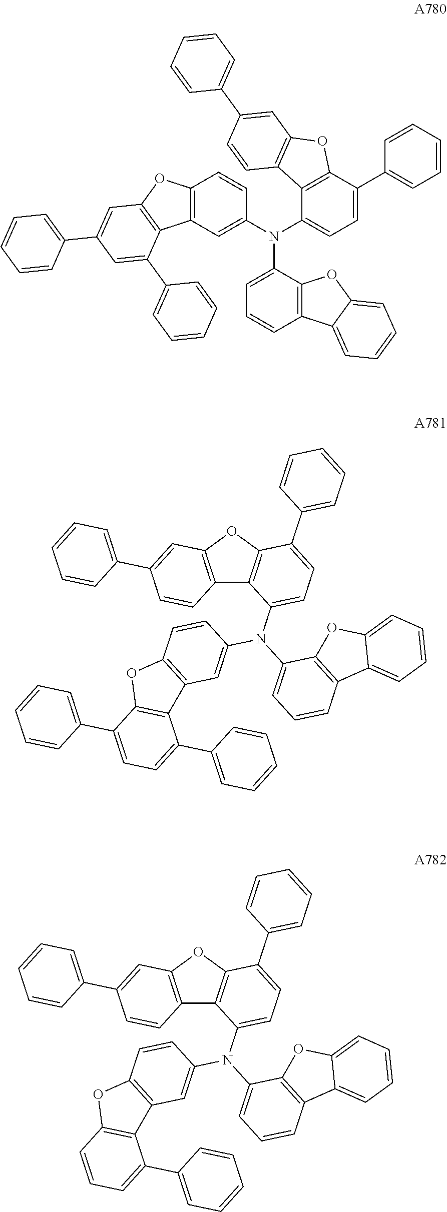 [SMILES: c1ccc(-c2ccc3c(c2)oc2c(-c4ccccc4)ccc(N(c4ccc5oc6cc(-c7ccccc7)cc(-c7ccccc7)c6c5c4)c4cccc5c4oc4ccccc45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5c4oc4ccccc45)c23)cc1 ; c1ccc(-c2ccc3c(c2)oc2c(-c4ccccc4)ccc(N(c4ccc5oc6cccc(-c7ccccc7)c6c5c4)c4cccc5c4oc4ccccc45)c23)cc1]

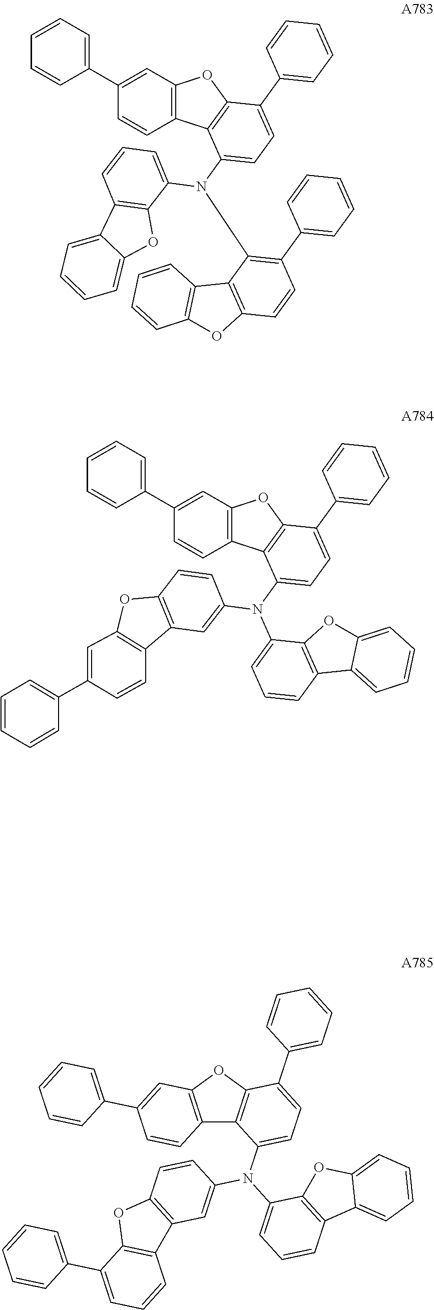 [SMILES: c1ccc(-c2ccc3c(c2)oc2c(-c4ccccc4)ccc(N(c4cccc5c4oc4ccccc45)c4c(-c5ccccc5)ccc5oc6ccccc6c45)c23)cc1 ; c1ccc(-c2ccc3c(c2)oc2ccc(N(c4cccc5c4oc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4cccc5c4oc4ccccc45)c23)cc1]

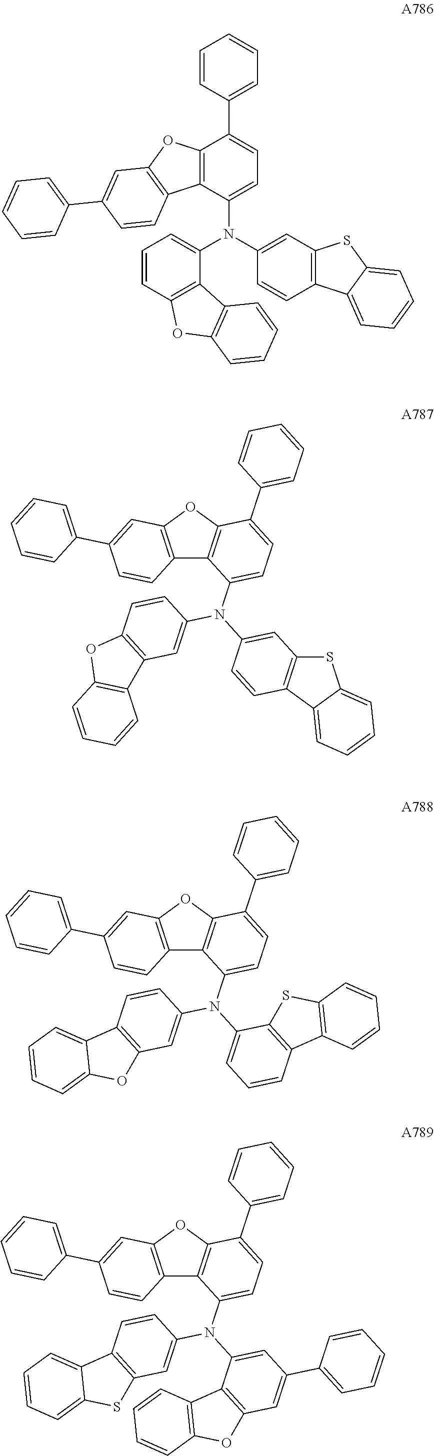 [SMILES: c1ccc(-c2ccc3c(c2)oc2c(-c4ccccc4)ccc(N(c4ccc5c(c4)sc4ccccc45)c4cccc5oc6ccccc6c45)c23)cc1 ; c1ccc(-c2ccc3c(c2)oc2c(-c4ccccc4)ccc(N(c4ccc5c(c4)sc4ccccc45)c4ccc5oc6ccccc6c5c4)c23)cc1 ; c1ccc(-c2ccc3c(c2)oc2c(-c4ccccc4)ccc(N(c4ccc5c(c4)oc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5oc6ccccc6c45)c23)cc1]

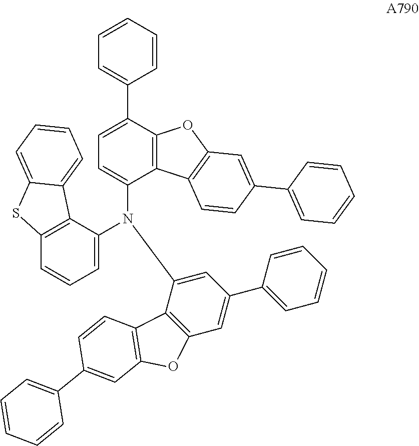 [SMILES: c1ccc(-c2ccc3c(c2)oc2cc(-c4ccccc4)cc(N(c4cccc5sc6ccccc6c45)c4ccc(-c5ccccc5)c5oc6cc(-c7ccccc7)ccc6c45)c23)cc1]

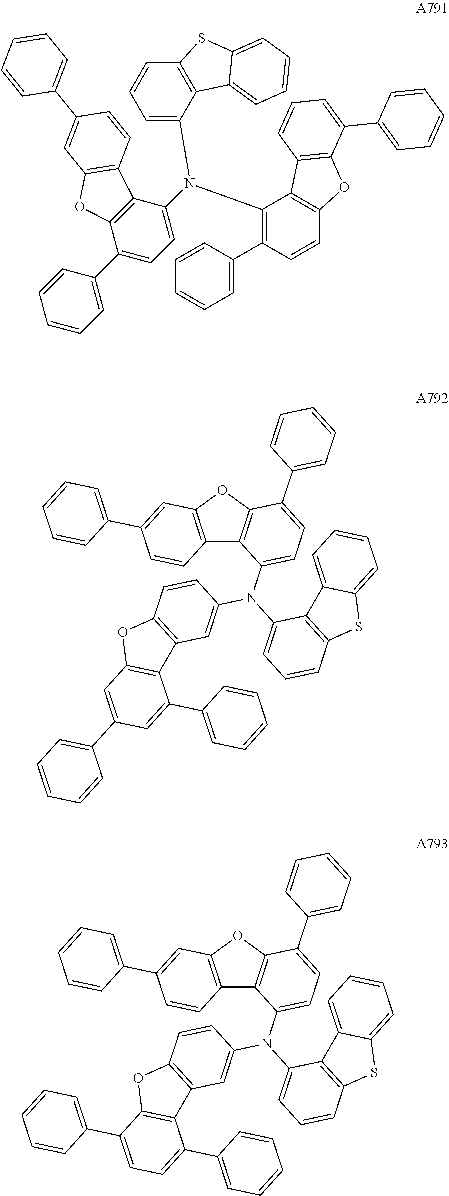 [SMILES: c1ccc(-c2ccc3c(c2)oc2c(-c4ccccc4)ccc(N(c4c(-c5ccccc5)ccc5oc6c(-c7ccccc7)cccc6c45)c4cccc5sc6ccccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5sc6ccccc6c45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5sc6ccccc6c45)c23)cc1]

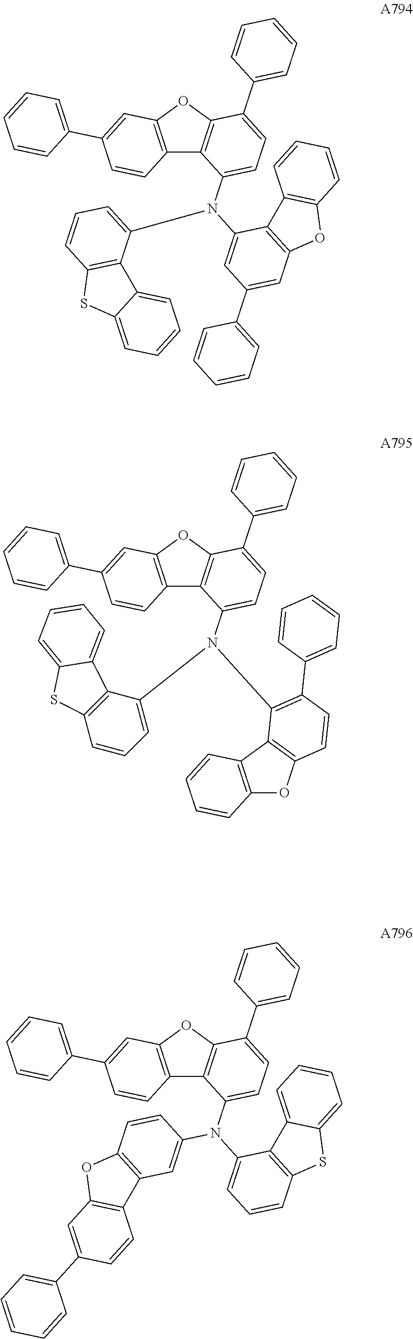 [SMILES: c1ccc(-c2ccc3c(c2)oc2c(-c4ccccc4)ccc(N(c4cc(-c5ccccc5)cc5oc6ccccc6c45)c4cccc5sc6ccccc6c45)c23)cc1 ; c1ccc(-c2ccc3c(c2)oc2c(-c4ccccc4)ccc(N(c4c(-c5ccccc5)ccc5oc6ccccc6c45)c4cccc5sc6ccccc6c45)c23)cc1 ; c1ccc(-c2ccc3c(c2)oc2ccc(N(c4cccc5sc6ccccc6c45)c4ccc(-c5ccccc5)c5oc6cc(-c7ccccc7)ccc6c45)cc23)cc1]

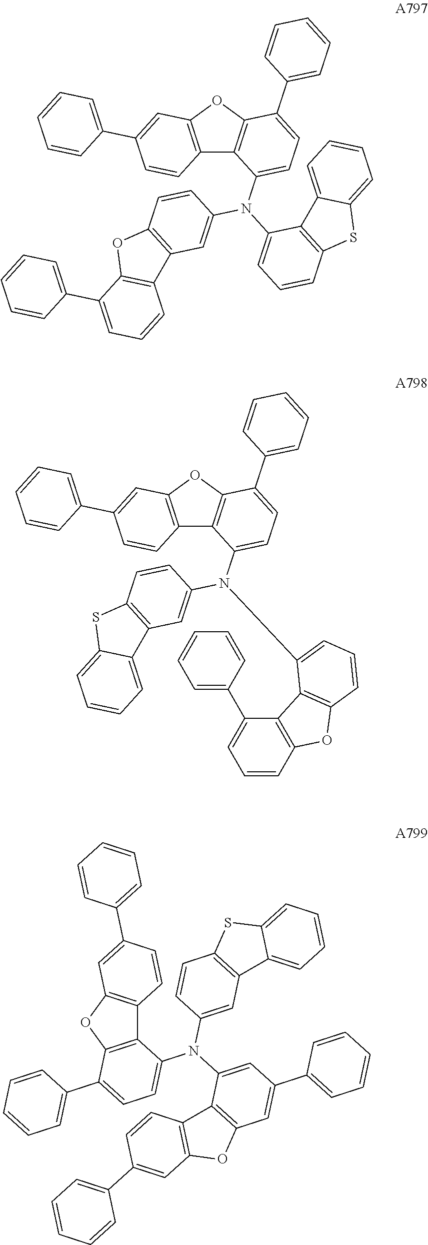 [SMILES: c1ccc(-c2ccc3c(c2)oc2c(-c4ccccc4)ccc(N(c4ccc5oc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5sc6ccccc6c5c4)c4ccc(-c5ccccc5)c5oc6cc(-c7ccccc7)ccc6c45)c23)cc1]

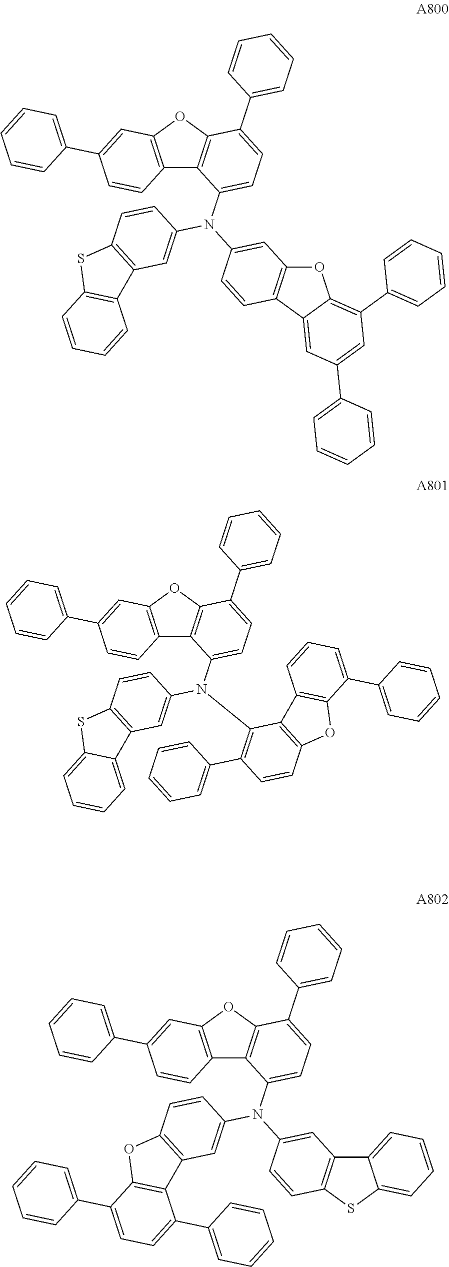 [SMILES: c1ccc(-c2ccc3c(c2)oc2c(-c4ccccc4)ccc(N(c4ccc5c(c4)oc4c(-c6ccccc6)cc(-c6ccccc6)cc45)c4ccc5sc6ccccc6c5c4)c23)cc1 ; c1ccc(-c2ccc3c(c2)oc2c(-c4ccccc4)ccc(N(c4ccc5sc6ccccc6c5c4)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5sc6ccccc6c5c4)c4ccc5oc6c(-c7ccccc7)ccc(-c7ccccc7)c6c5c4)c23)cc1]

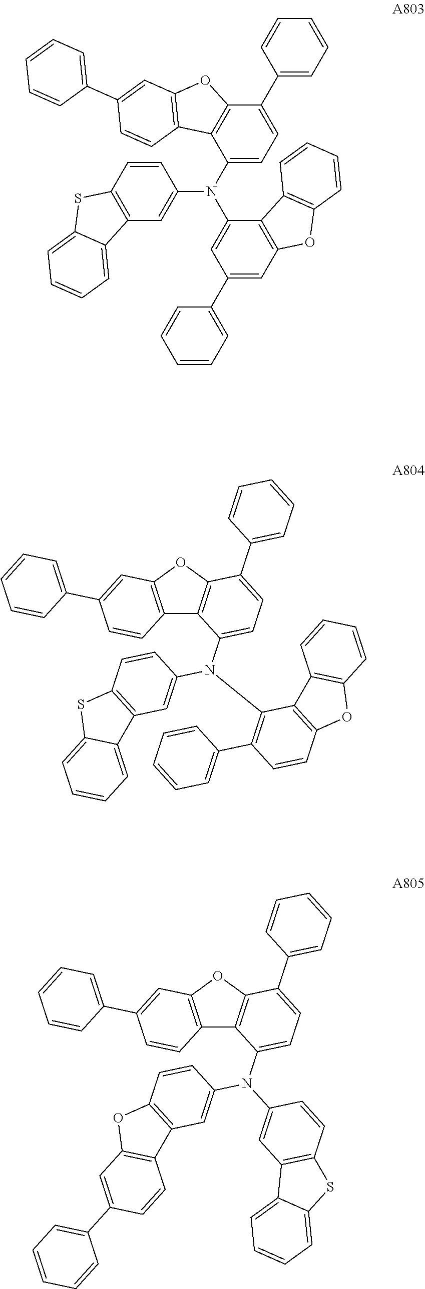 [SMILES: c1ccc(-c2ccc3c(c2)oc2c(-c4ccccc4)ccc(N(c4ccc5sc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc5sc6ccccc6c5c4)c4c(-c5ccccc5)ccc5oc6ccccc6c45)c23)cc1 ; c1ccc(-c2ccc3c(c2)oc2ccc(N(c4ccc5sc6ccccc6c5c4)c4ccc(-c5ccccc5)c5oc6cc(-c7ccccc7)ccc6c45)cc23)cc1]

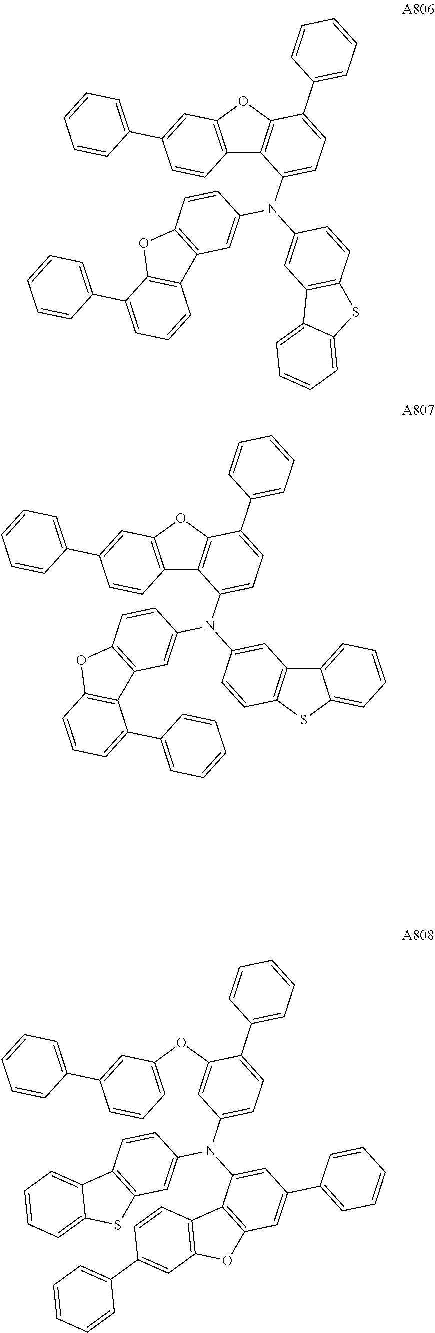 [SMILES: c1ccc(-c2ccc3c(c2)oc2c(-c4ccccc4)ccc(N(c4ccc5oc6c(-c7ccccc7)cccc6c5c4)c4ccc5sc6ccccc6c5c4)c23)cc1 ; c1ccc(-c2ccc3c(c2)oc2c(-c4ccccc4)ccc(N(c4ccc5sc6ccccc6c5c4)c4ccc5oc6cccc(-c7ccccc7)c6c5c4)c23)cc1 ; c1ccc(-c2cccc(Oc3cc(N(c4ccc5c(c4)sc4ccccc45)c4cc(-c5ccccc5)cc5oc6cc(-c7ccccc7)ccc6c45)ccc3-c3ccccc3)c2)cc1]

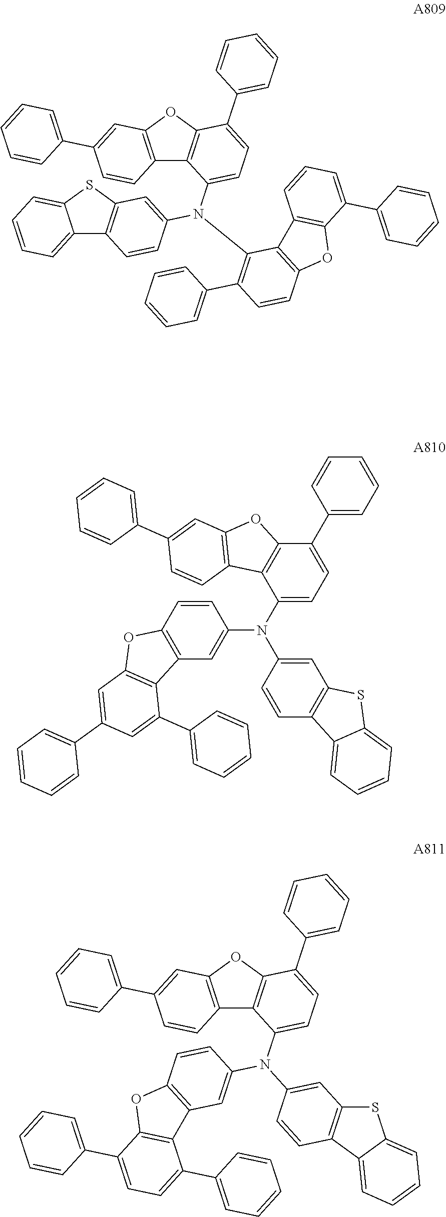 [SMILES: c1ccc(-c2ccc3c(c2)oc2c(-c4ccccc4)ccc(N(c4ccc5c(c4)sc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5oc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5oc6c(-c7ccccc7)ccc(-c7ccccc7)c6c5c4)c23)cc1]

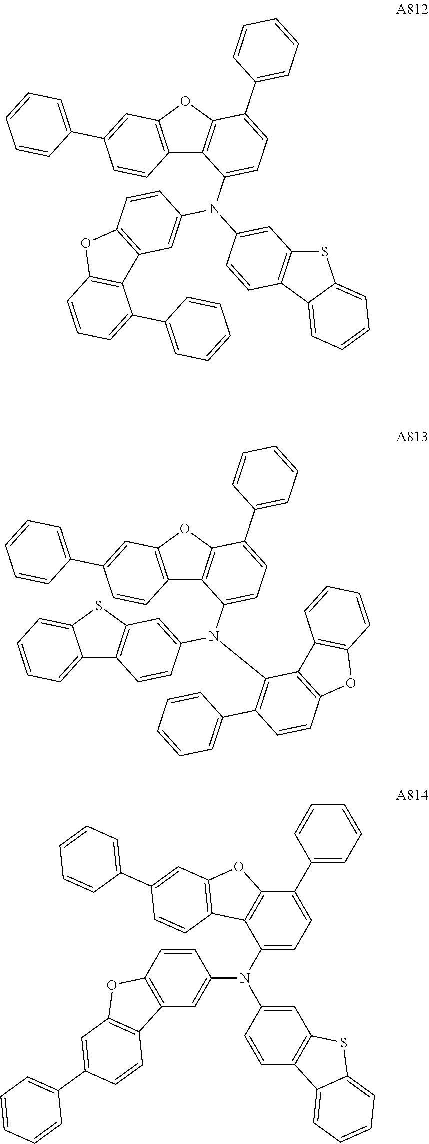 [SMILES: c1ccc(-c2ccc3c(c2)oc2c(-c4ccccc4)ccc(N(c4ccc5c(c4)sc4ccccc45)c4ccc5oc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5oc6ccccc6c45)c23)cc1 ; c1ccc(-c2ccc3c(c2)oc2ccc(N(c4ccc5c(c4)sc4ccccc45)c4ccc(-c5ccccc5)c5oc6cc(-c7ccccc7)ccc6c45)cc23)cc1]

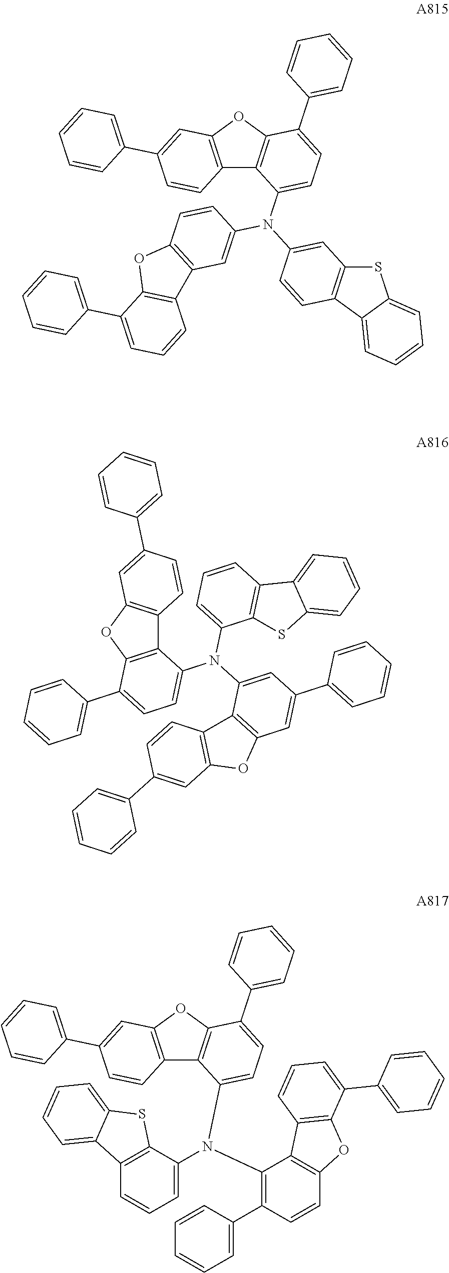 [SMILES: c1ccc(-c2ccc3c(c2)oc2c(-c4ccccc4)ccc(N(c4ccc5c(c4)sc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4sc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4cccc5c4sc4ccccc45)c4c(-c5ccccc5)ccc5oc6c(-c7ccccc7)cccc6c45)c23)cc1]

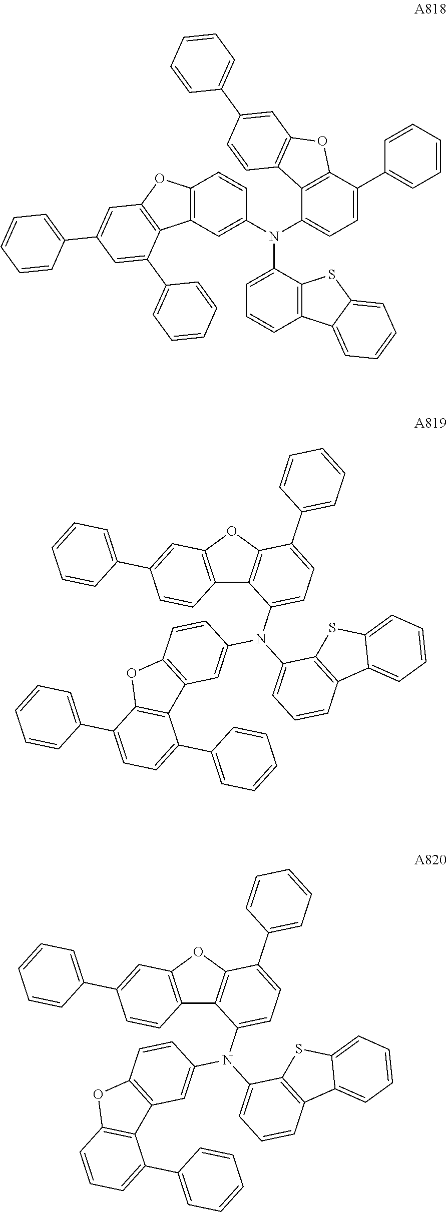 [SMILES: c1ccc(-c2ccc3c(c2)oc2c(-c4ccccc4)ccc(N(c4ccc5oc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5c4sc4ccccc45)c23)cc1 ; c1ccc(-c2ccc3c(c2)oc2c(-c4ccccc4)ccc(N(c4ccc5oc6cccc(-c7ccccc7)c6c5c4)c4cccc5c4sc4ccccc45)c23)cc1]

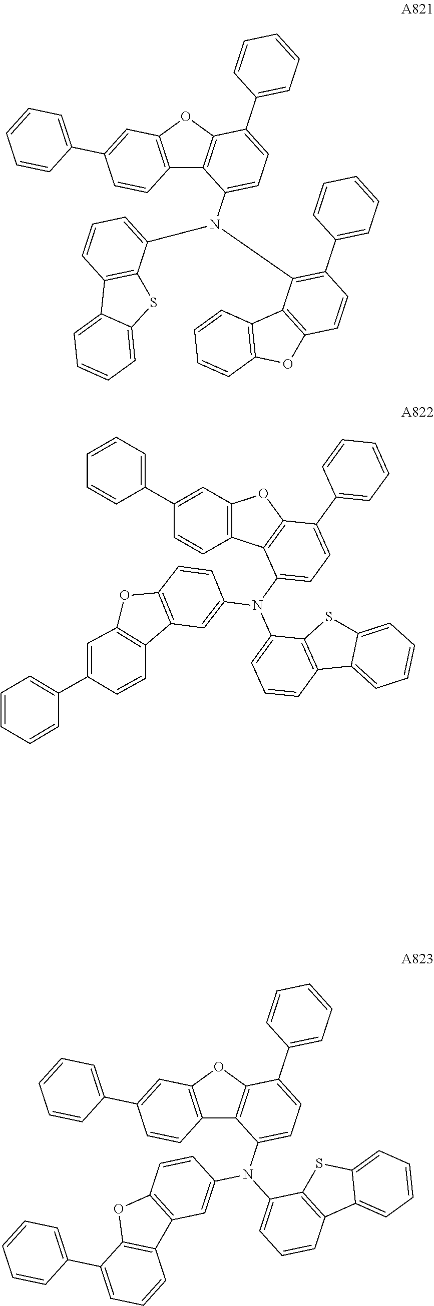 [SMILES: c1ccc(-c2ccc3c(c2)oc2c(-c4ccccc4)ccc(N(c4cccc5c4sc4ccccc45)c4c(-c5ccccc5)ccc5oc6ccccc6c45)c23)cc1 ; c1ccc(-c2ccc3c(c2)oc2ccc(N(c4cccc5c4sc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4cccc5c4sc4ccccc45)c23)cc1]

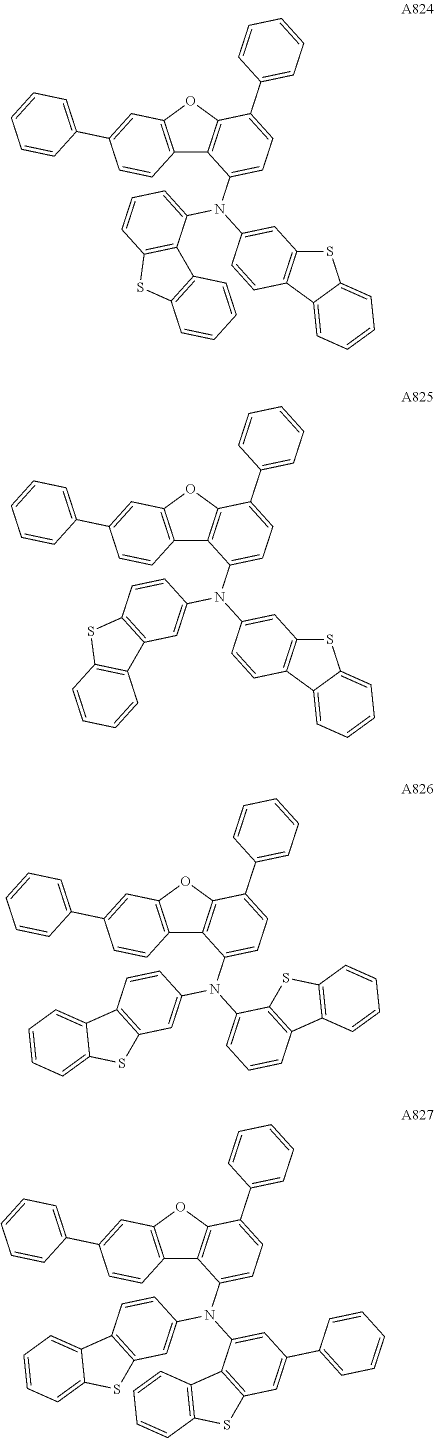 [SMILES: c1ccc(-c2ccc3c(c2)oc2c(-c4ccccc4)ccc(N(c4ccc5c(c4)sc4ccccc45)c4cccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6ccccc6c5c4)c23)cc1 ; c1ccc(-c2ccc3c(c2)oc2c(-c4ccccc4)ccc(N(c4ccc5c(c4)sc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5sc6ccccc6c45)c23)cc1]

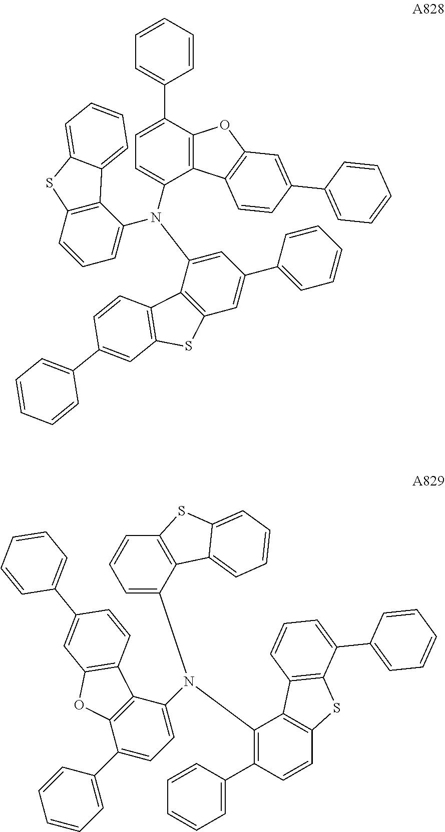 [SMILES: c1ccc(-c2ccc3c(c2)oc2c(-c4ccccc4)ccc(N(c4cccc5sc6ccccc6c45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5sc6ccccc6c45)c4c(-c5ccccc5)ccc5sc6c(-c7ccccc7)cccc6c45)c23)cc1]

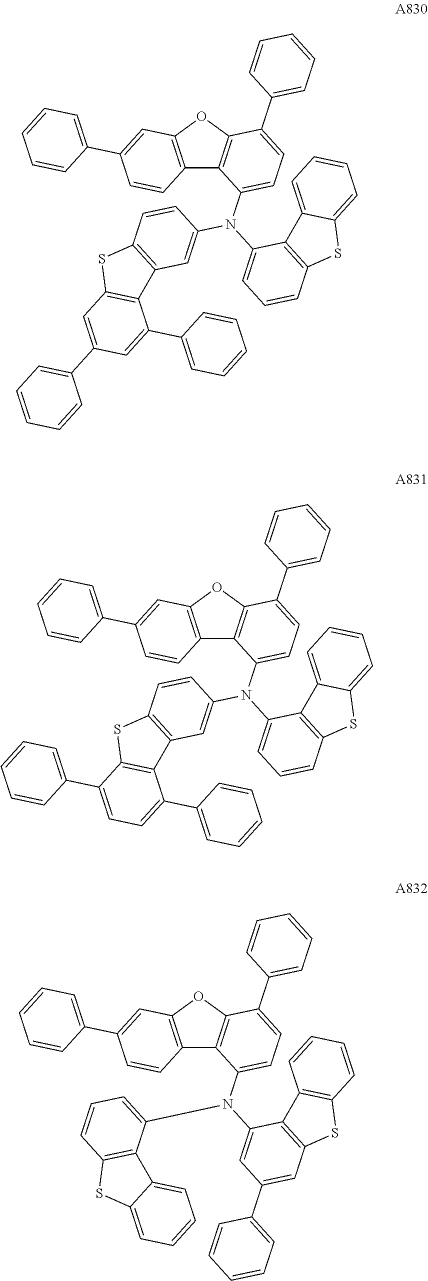 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5sc6ccccc6c45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5sc6ccccc6c45)c23)cc1 ; c1ccc(-c2ccc3c(c2)oc2c(-c4ccccc4)ccc(N(c4cccc5sc6ccccc6c45)c4cc(-c5ccccc5)cc5sc6ccccc6c45)c23)cc1]

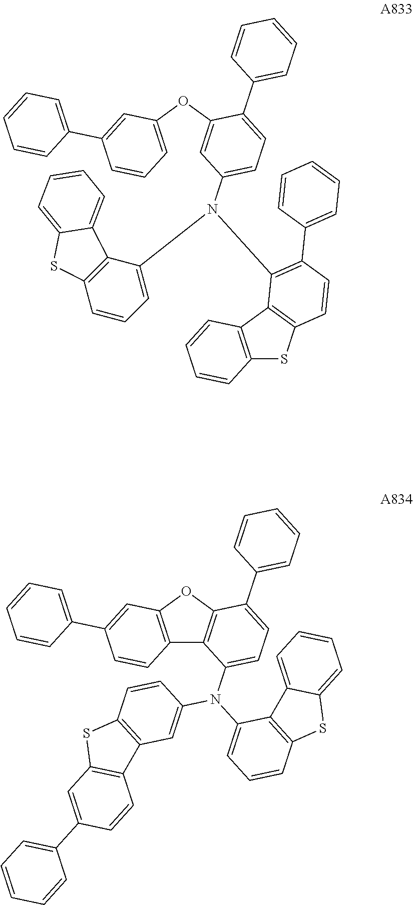 [SMILES: c1ccc(-c2cccc(Oc3cc(N(c4cccc5sc6ccccc6c45)c4c(-c5ccccc5)ccc5sc6ccccc6c45)ccc3-c3ccccc3)c2)cc1 ; c1ccc(-c2ccc3c(c2)oc2c(-c4ccccc4)ccc(N(c4ccc5sc6cc(-c7ccccc7)ccc6c5c4)c4cccc5sc6ccccc6c45)c23)cc1]

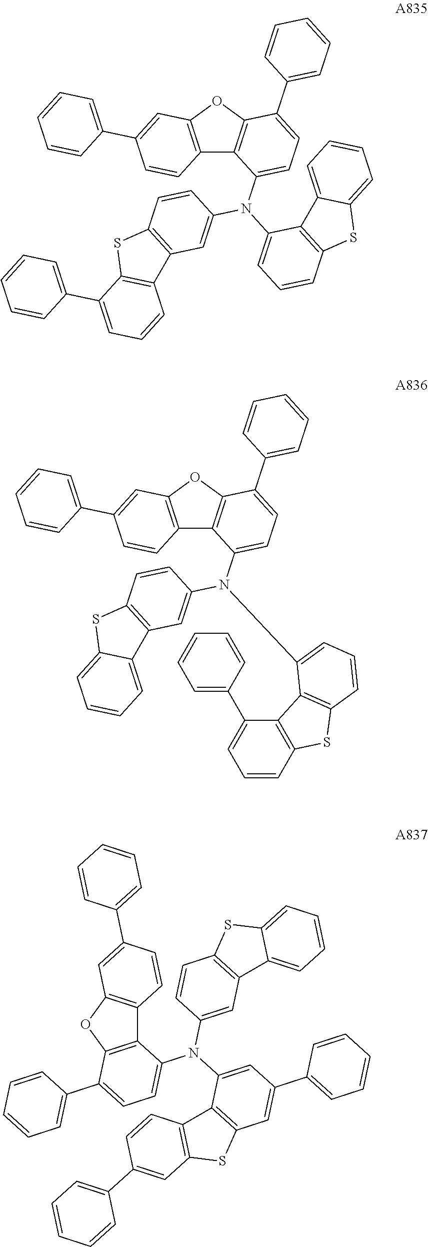 [SMILES: c1ccc(-c2ccc3c(c2)oc2c(-c4ccccc4)ccc(N(c4ccc5sc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5sc6cccc(-c7ccccc7)c6c45)c23)cc1 ; c1ccc(-c2ccc3c(c2)oc2c(-c4ccccc4)ccc(N(c4ccc5sc6ccccc6c5c4)c4cc(-c5ccccc5)cc5sc6cc(-c7ccccc7)ccc6c45)c23)cc1]

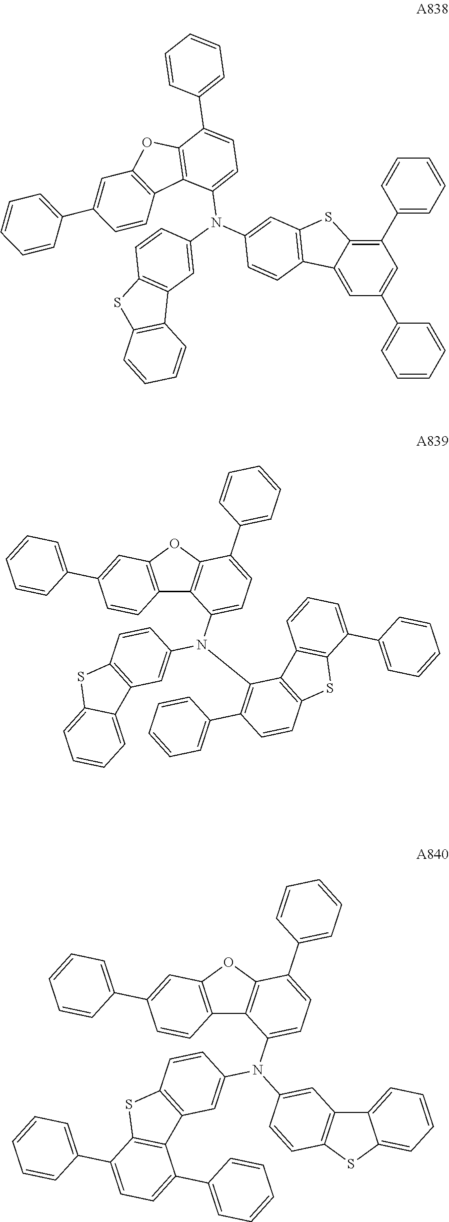 [SMILES: c1ccc(-c2ccc3c(c2)oc2c(-c4ccccc4)ccc(N(c4ccc5c(c4)sc4c(-c6ccccc6)cc(-c6ccccc6)cc45)c4ccc5sc6ccccc6c5c4)c23)cc1 ; c1ccc(-c2ccc3c(c2)oc2c(-c4ccccc4)ccc(N(c4ccc5sc6ccccc6c5c4)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6ccccc6c5c4)c4ccc5sc6c(-c7ccccc7)ccc(-c7ccccc7)c6c5c4)c23)cc1]

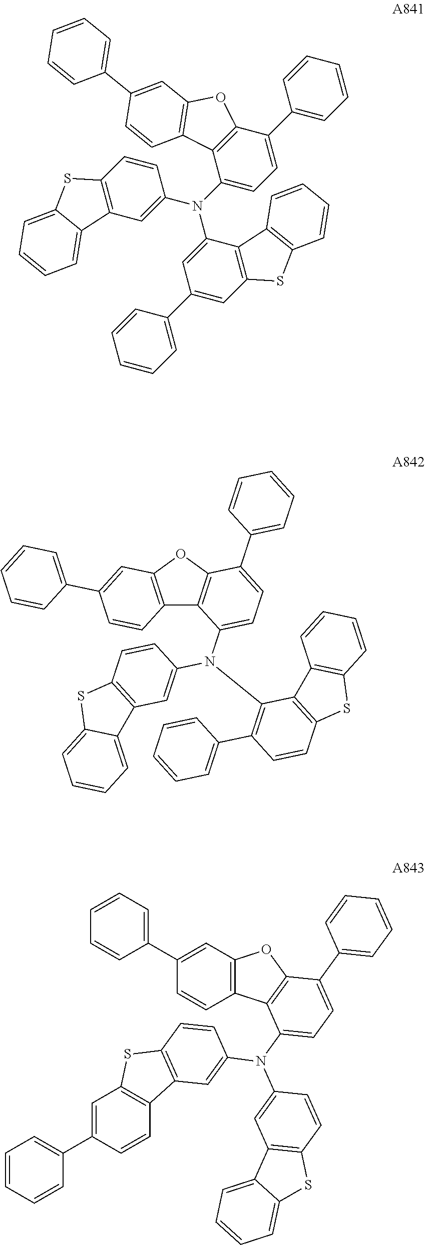 [SMILES: c1ccc(-c2ccc3c(c2)oc2c(-c4ccccc4)ccc(N(c4ccc5sc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc5sc6ccccc6c5c4)c4c(-c5ccccc5)ccc5sc6ccccc6c45)c23)cc1 ; c1ccc(-c2ccc3c(c2)oc2c(-c4ccccc4)ccc(N(c4ccc5sc6ccccc6c5c4)c4ccc5sc6cc(-c7ccccc7)ccc6c5c4)c23)cc1]

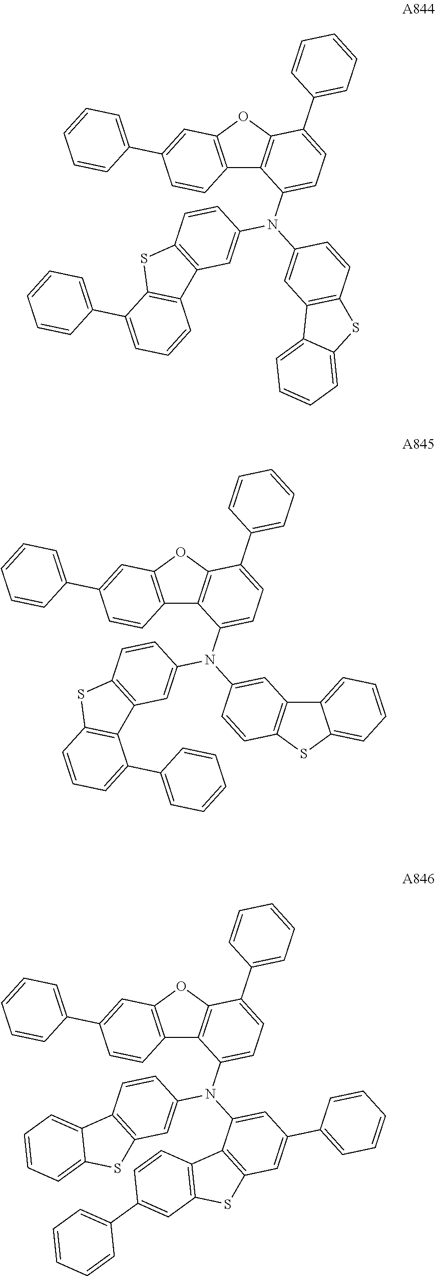 [SMILES: c1ccc(-c2ccc3c(c2)oc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)oc2c(-c4ccccc4)ccc(N(c4ccc5sc6ccccc6c5c4)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4cc(-c5ccccc5)cc5sc6cc(-c7ccccc7)ccc6c45)c23)cc1]

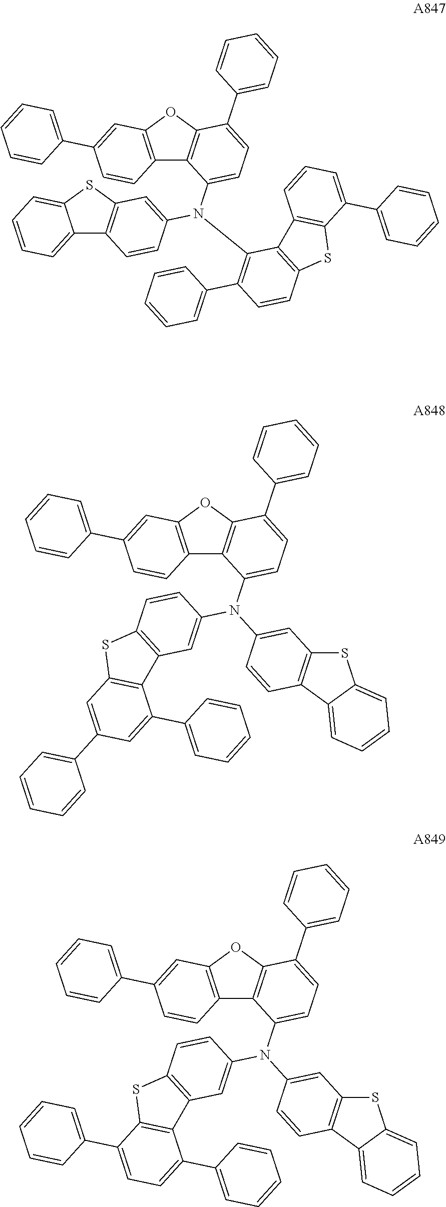 [SMILES: c1ccc(-c2ccc3c(c2)oc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5sc6c(-c7ccccc7)ccc(-c7ccccc7)c6c5c4)c23)cc1]

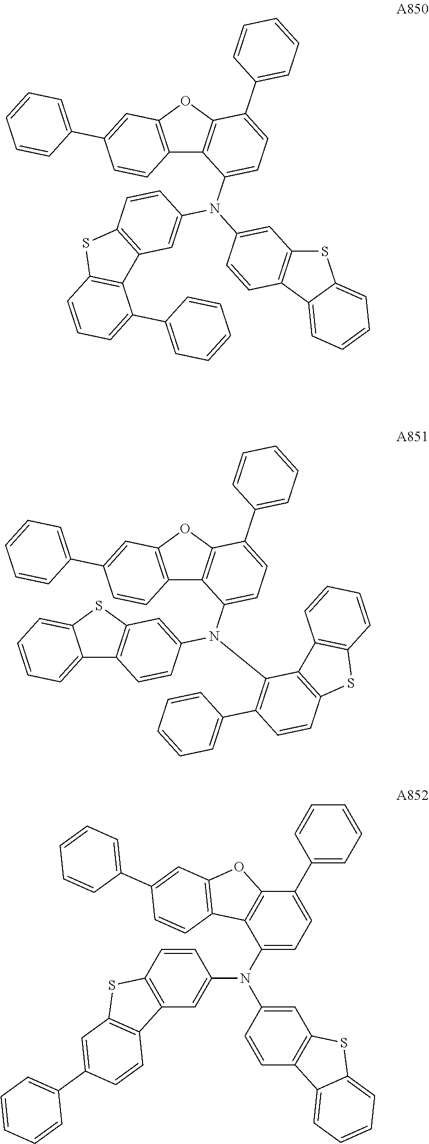 [SMILES: c1ccc(-c2ccc3c(c2)oc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)ccc6c5c4)c23)cc1]

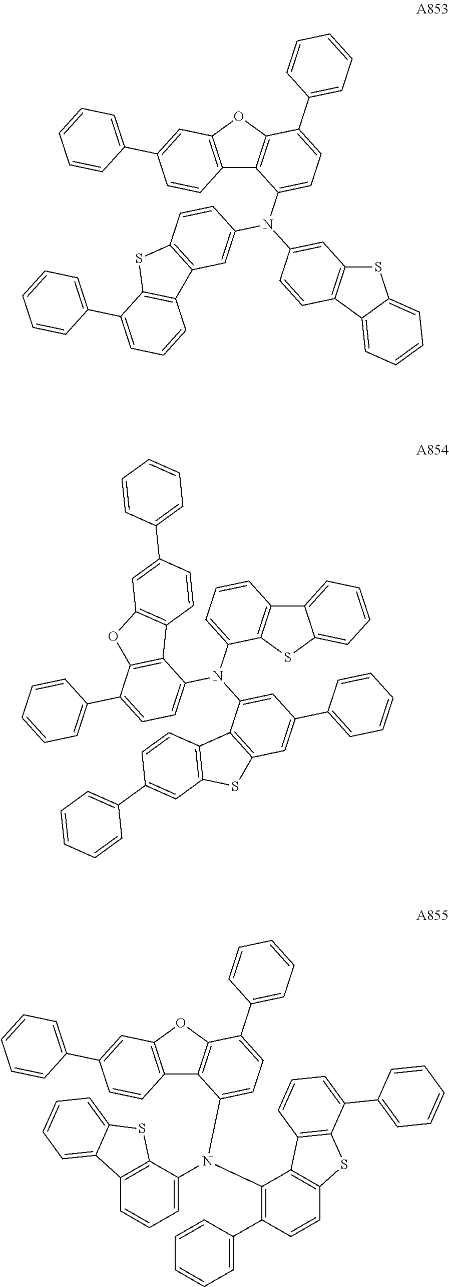 [SMILES: c1ccc(-c2ccc3c(c2)oc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)oc2c(-c4ccccc4)ccc(N(c4cccc5c4sc4ccccc45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5c4sc4ccccc45)c4c(-c5ccccc5)ccc5sc6c(-c7ccccc7)cccc6c45)c23)cc1]

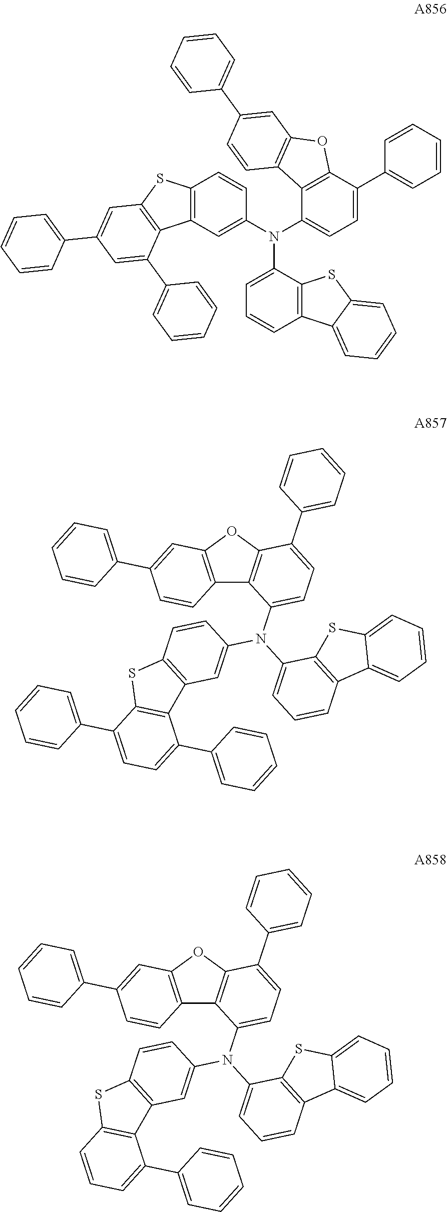 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6cccc(-c7ccccc7)c6c5c4)c4cccc5c4sc4ccccc45)c23)cc1]

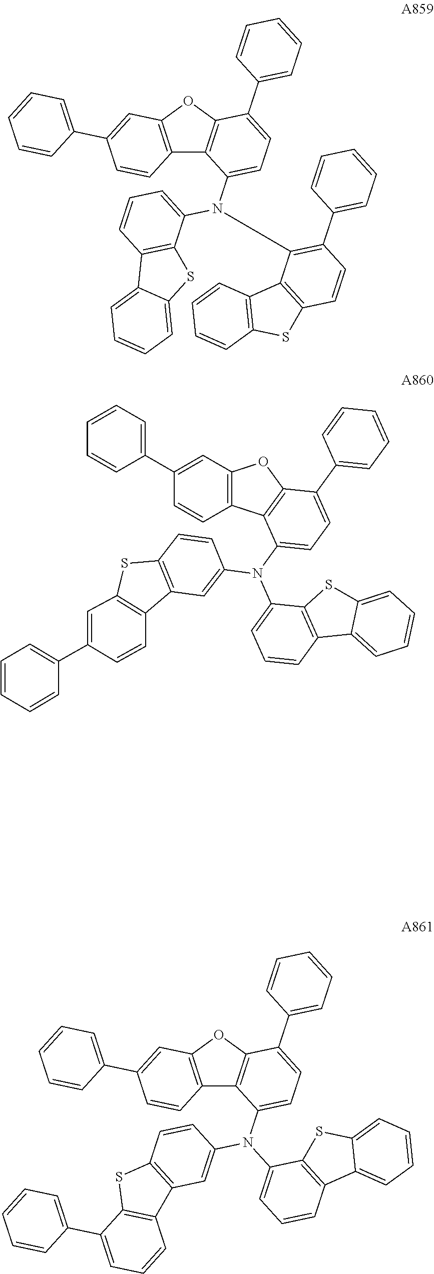 [SMILES: c1ccc(-c2ccc3c(c2)oc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5sc6cc(-c7ccccc7)ccc6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)cccc6c5c4)c4cccc5c4sc4ccccc45)c23)cc1]

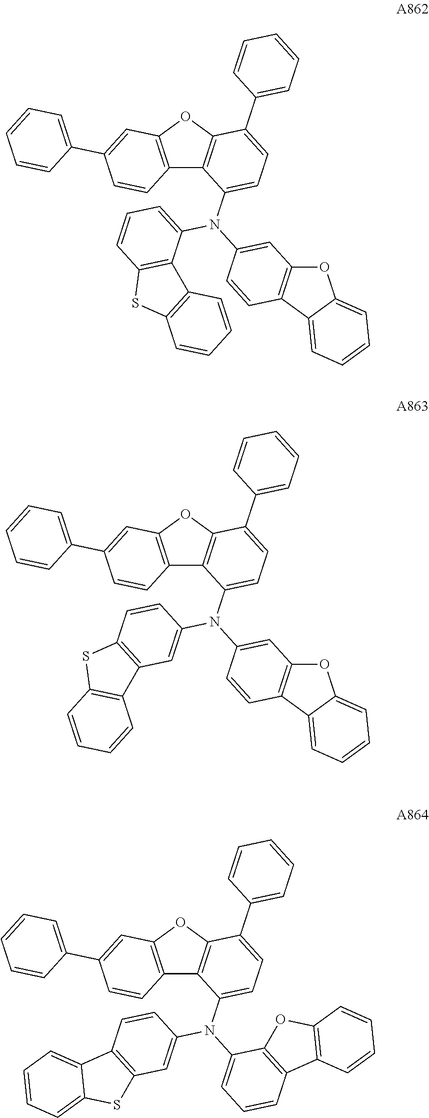 [SMILES: c1ccc(-c2ccc3c(c2)oc2c(-c4ccccc4)ccc(N(c4ccc5c(c4)oc4ccccc45)c4cccc5sc6ccccc6c45)c23)cc1 ; c1ccc(-c2ccc3c(c2)oc2c(-c4ccccc4)ccc(N(c4ccc5c(c4)oc4ccccc45)c4ccc5sc6ccccc6c5c4)c23)cc1 ; c1ccc(-c2ccc3c(c2)oc2c(-c4ccccc4)ccc(N(c4ccc5c(c4)sc4ccccc45)c4cccc5c4oc4ccccc45)c23)cc1]

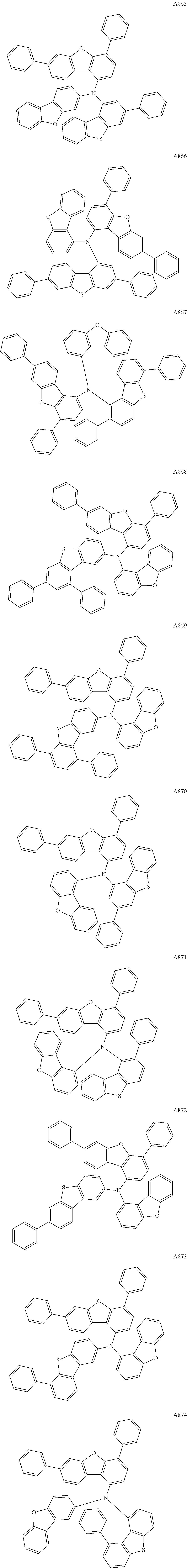

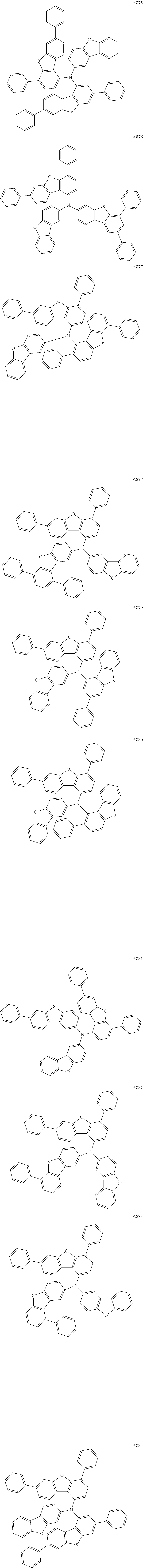

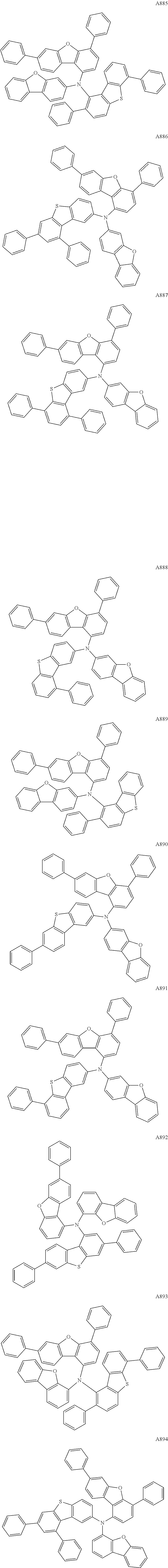

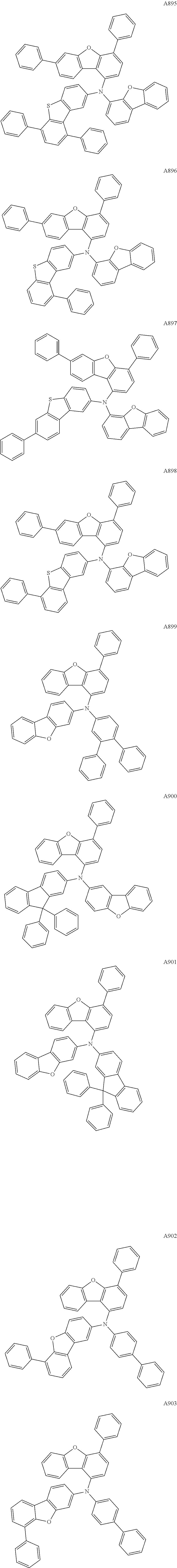

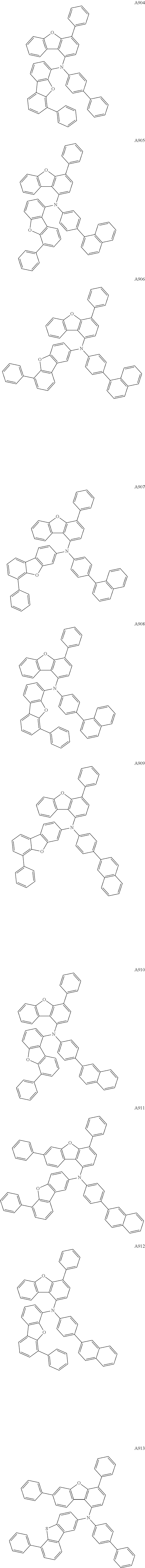

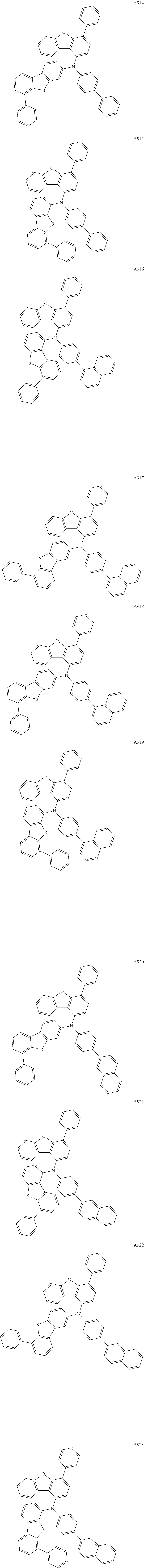

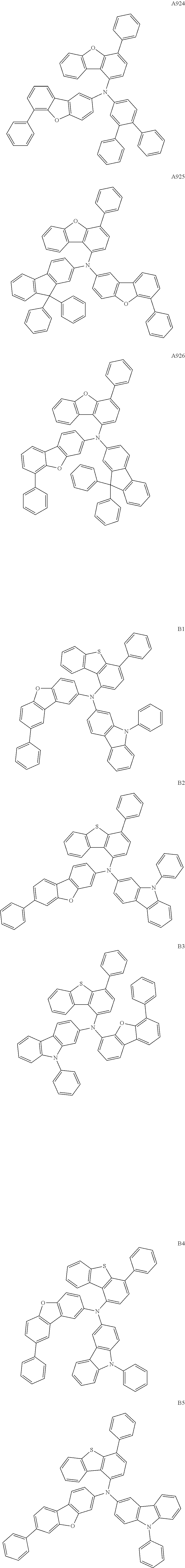

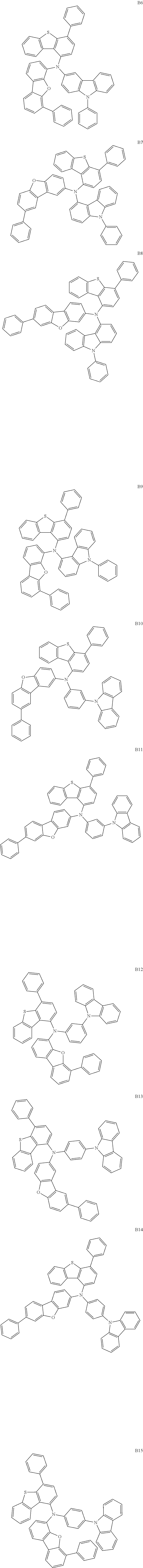

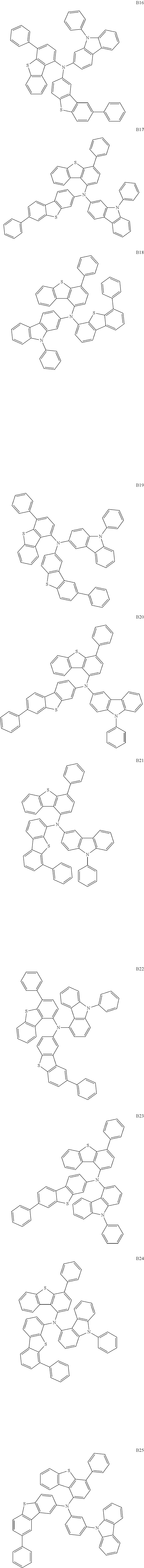

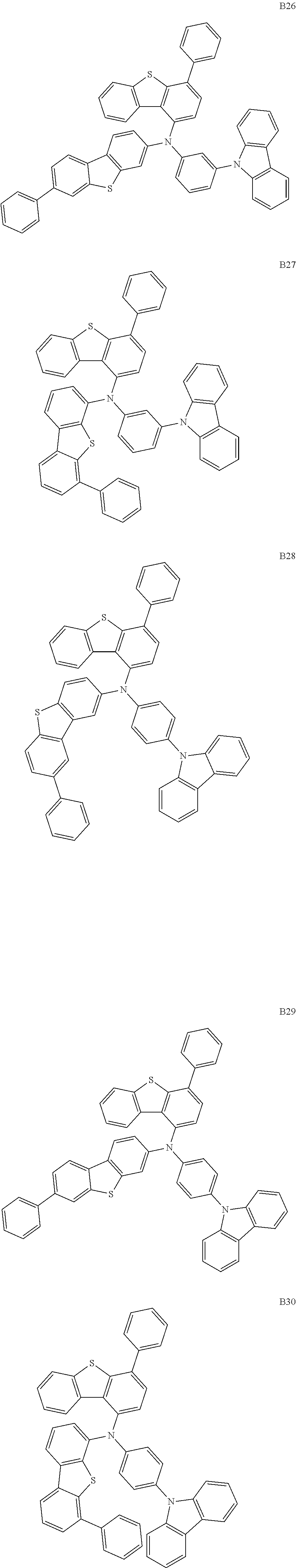 [SMILES: c1ccc(-c2ccc3c(c2)sc2cc(N(c4cccc(-n5c6ccccc6c6ccccc65)c4)c4ccc(-c5ccccc5)c5sc6ccccc6c45)ccc23)cc1 ; c1ccc(-c2cccc3c2sc2c(N(c4cccc(-n5c6ccccc6c6ccccc65)c4)c4ccc(-c5ccccc5)c5sc6ccccc6c45)cccc23)cc1 ; c1ccc(-c2ccc3sc4ccc(N(c5ccc(-n6c7ccccc7c7ccccc76)cc5)c5ccc(-c6ccccc6)c6sc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5sc6ccccc6c45)ccc23)cc1 ; c1ccc(-c2cccc3c2sc2c(N(c4ccc(-n5c6ccccc6c6ccccc65)cc4)c4ccc(-c5ccccc5)c5sc6ccccc6c45)cccc23)cc1]

[chemical structure]

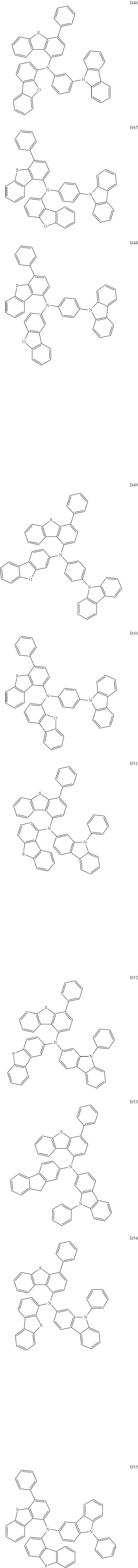

[chemical structure]

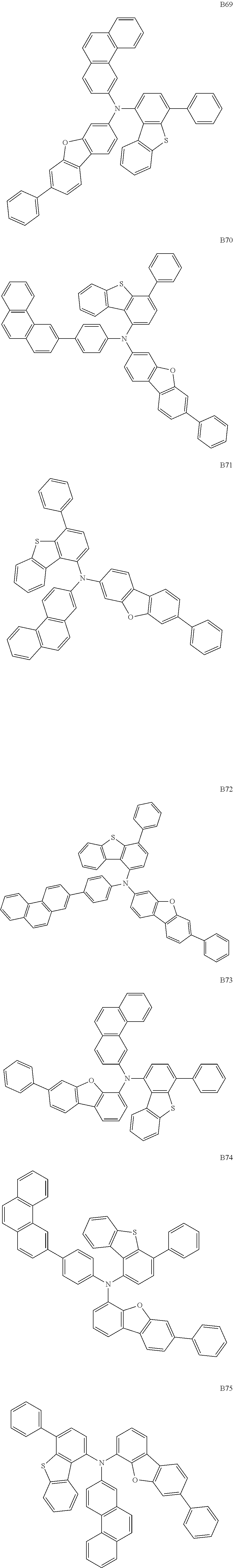

[chemical structure]

[chemical structure]

[chemical structure]

[chemical structure]

[chemical structure]

[chemical structure]

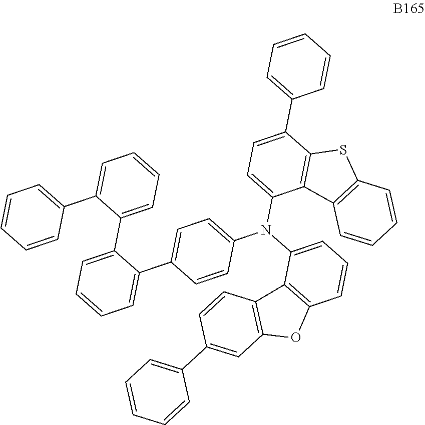 [SMILES: c1ccc(-c2ccc3c(c2)oc2cccc(N(c4ccc(-c5ccccc5-c5ccccc5-c5ccccc5)cc4)c4ccc(-c5ccccc5)c5sc6ccccc6c45)c23)cc1]

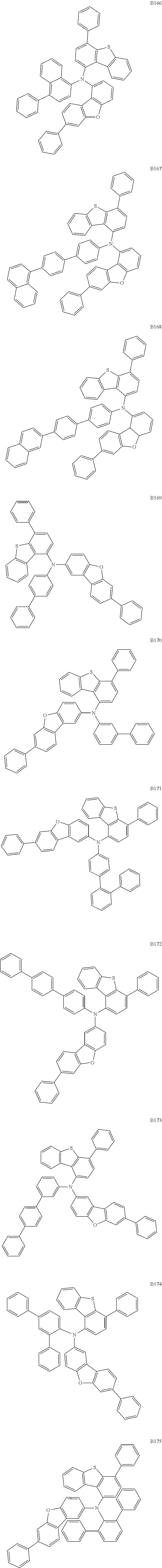

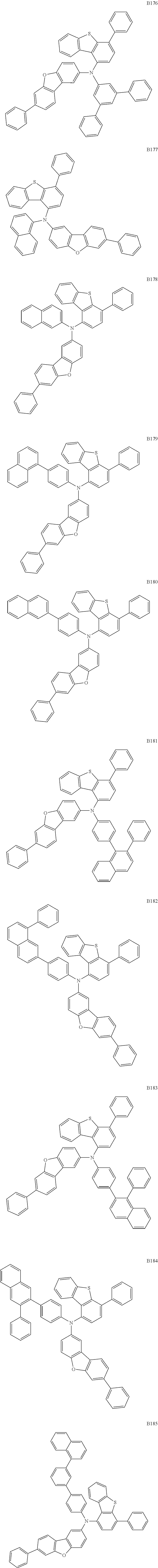

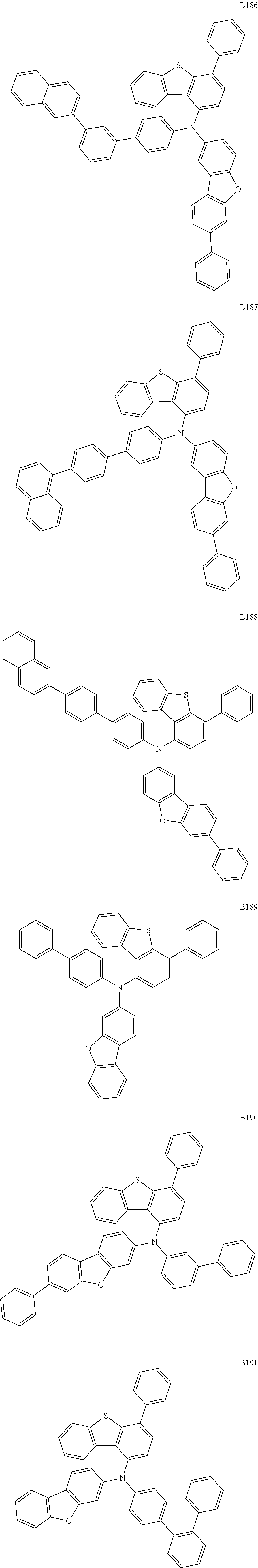

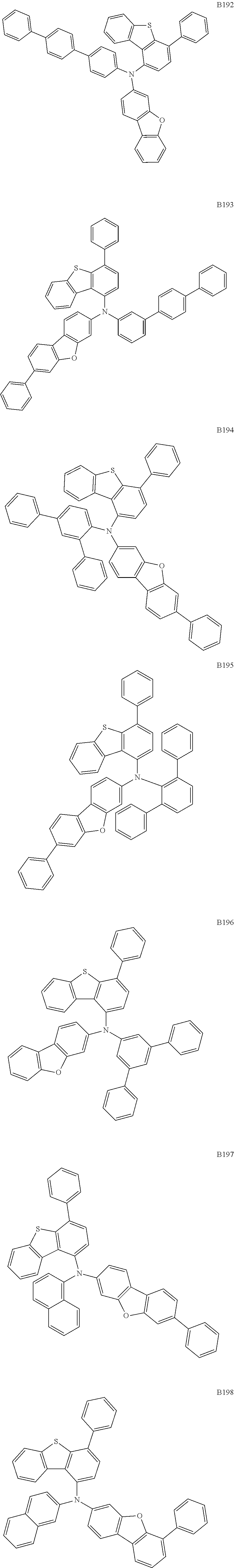

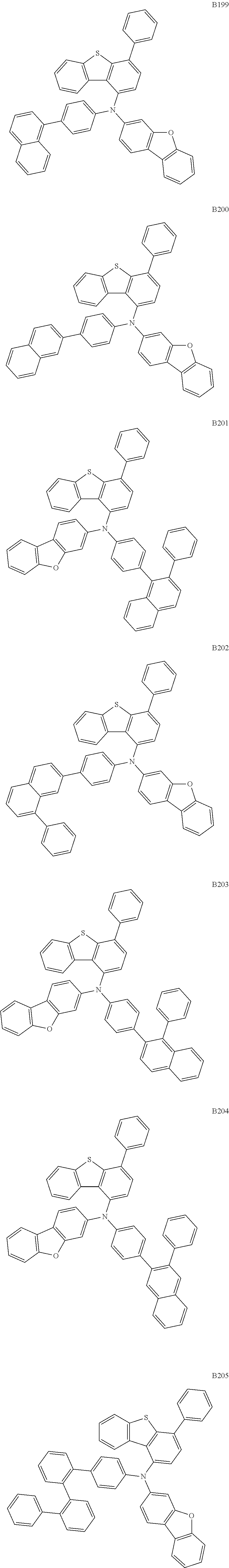

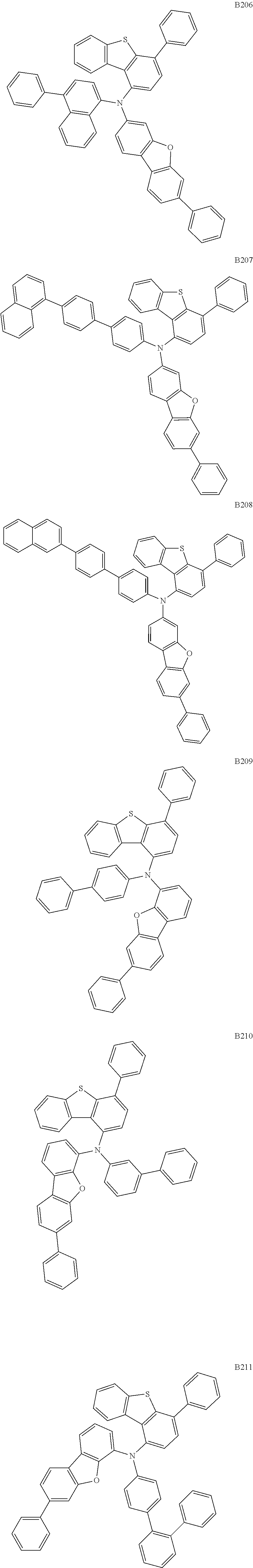

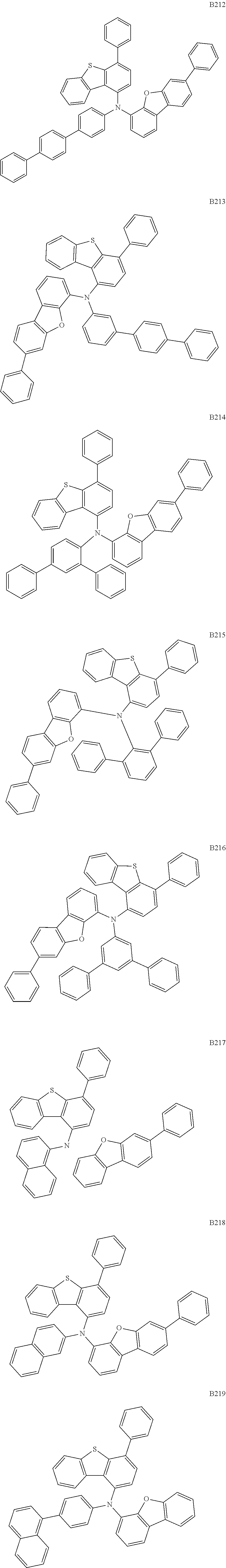

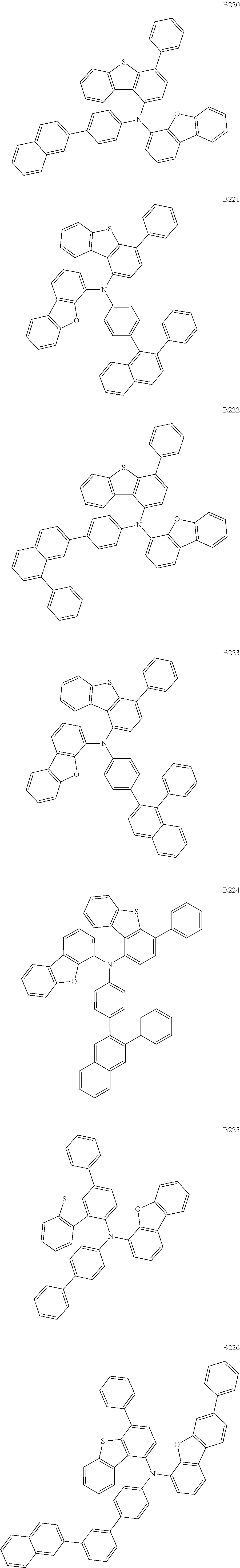

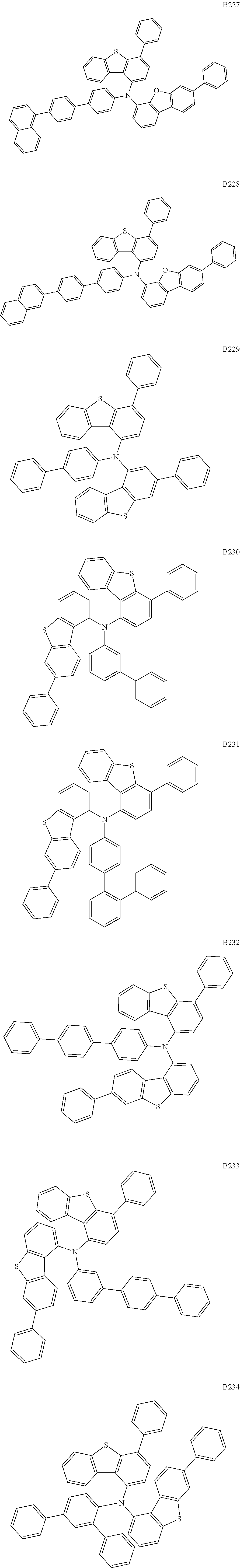

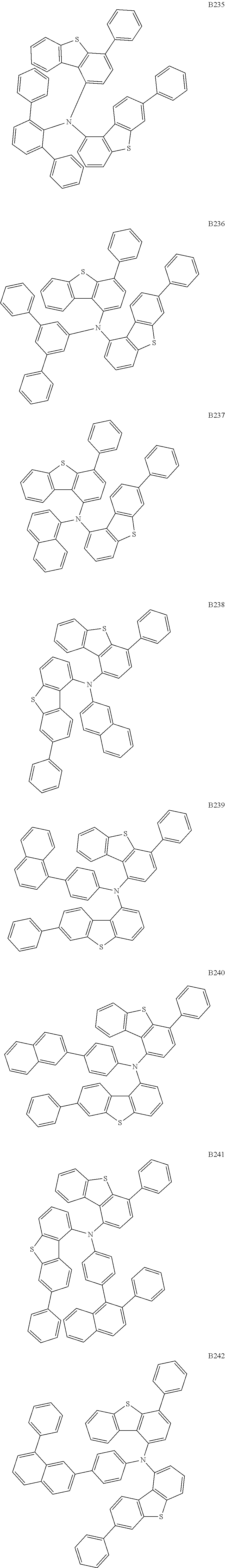

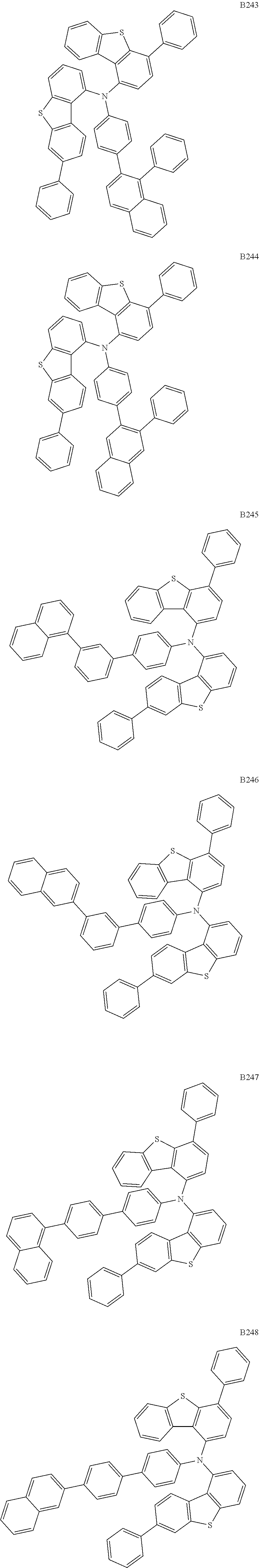

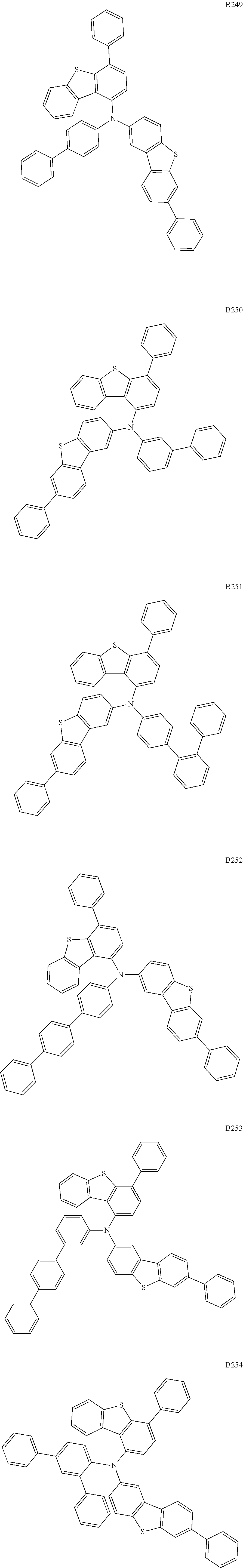

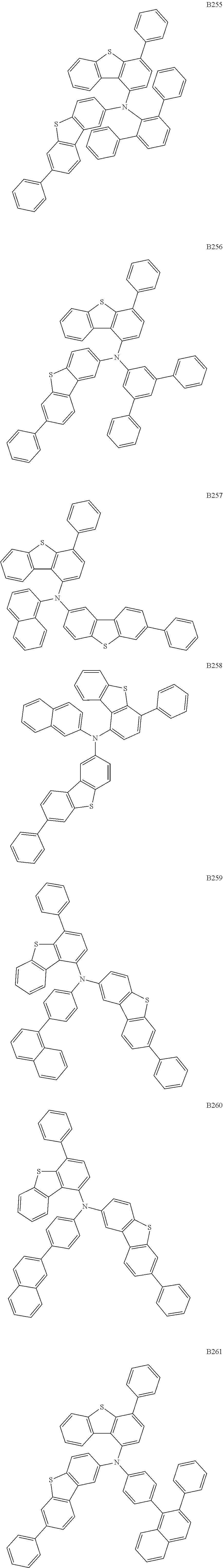

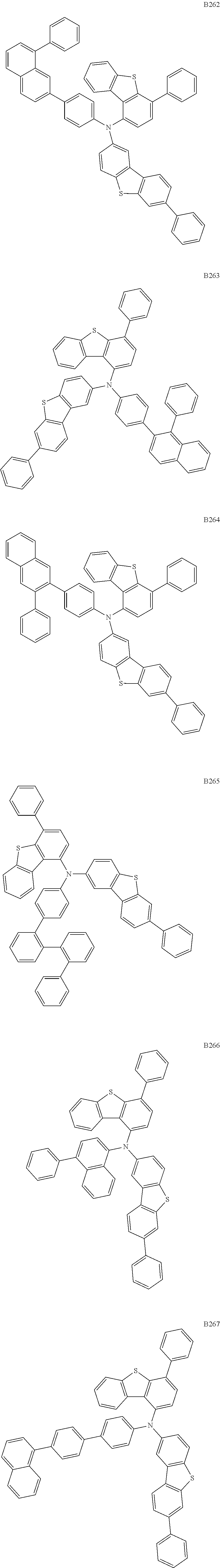

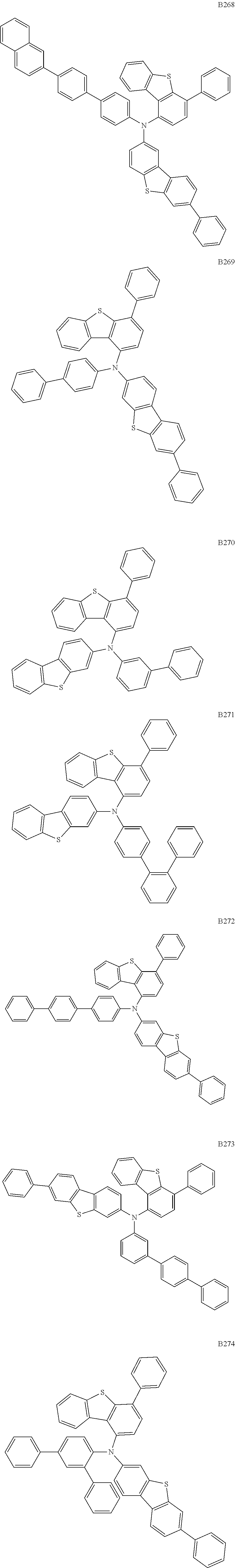

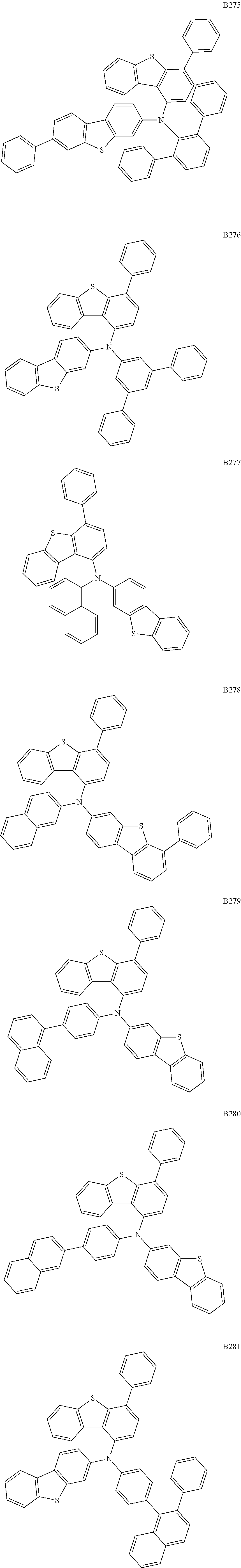

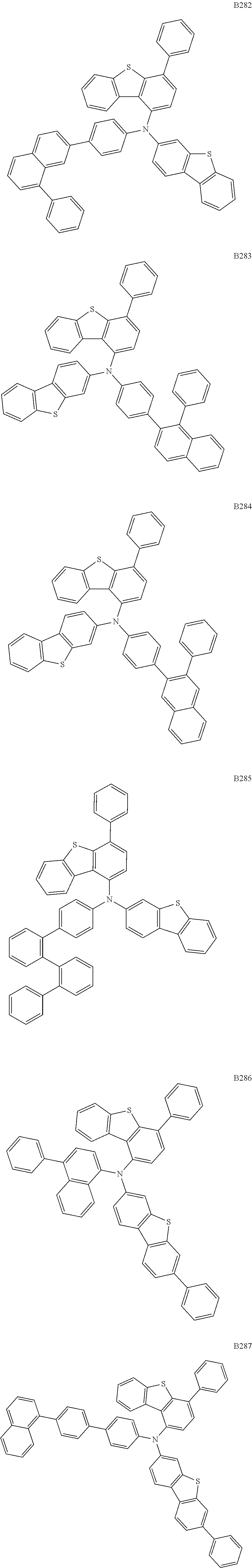

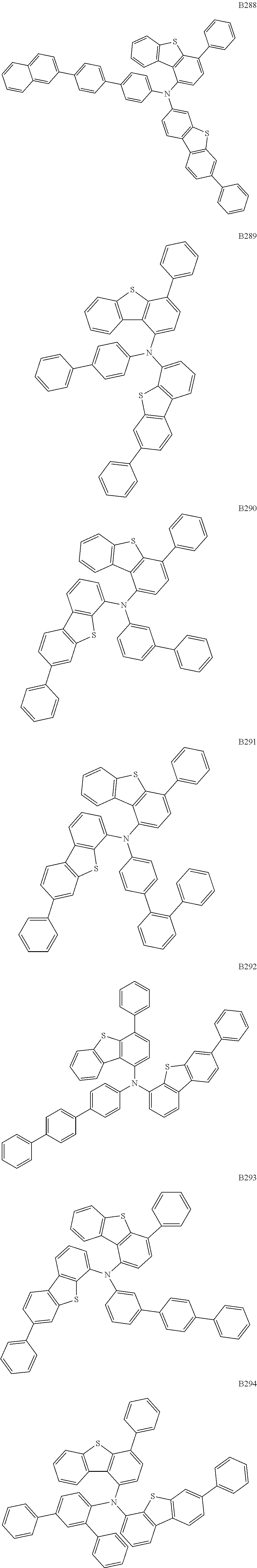

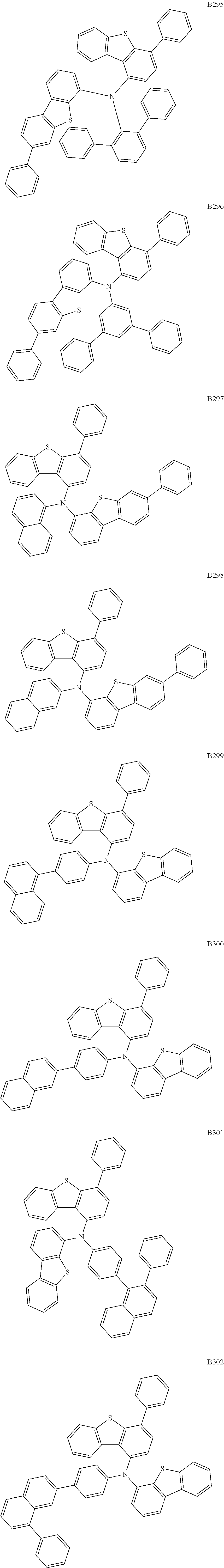

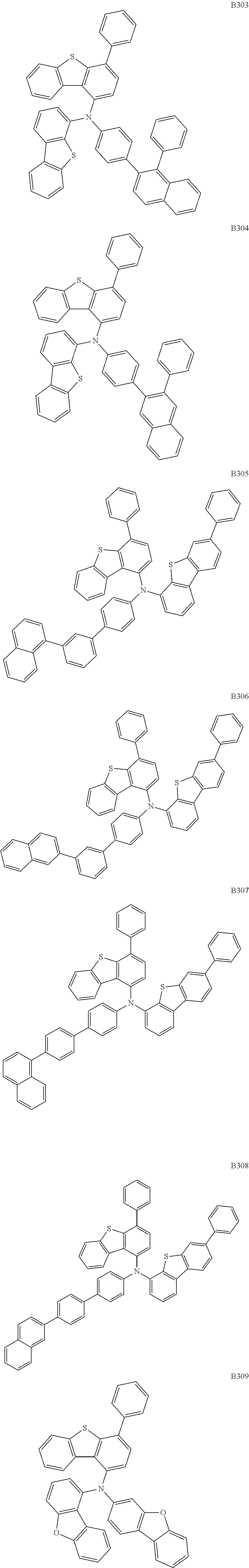

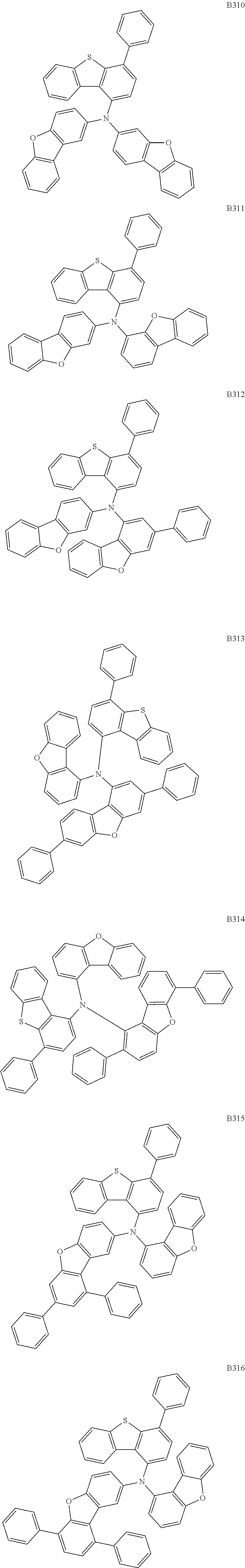

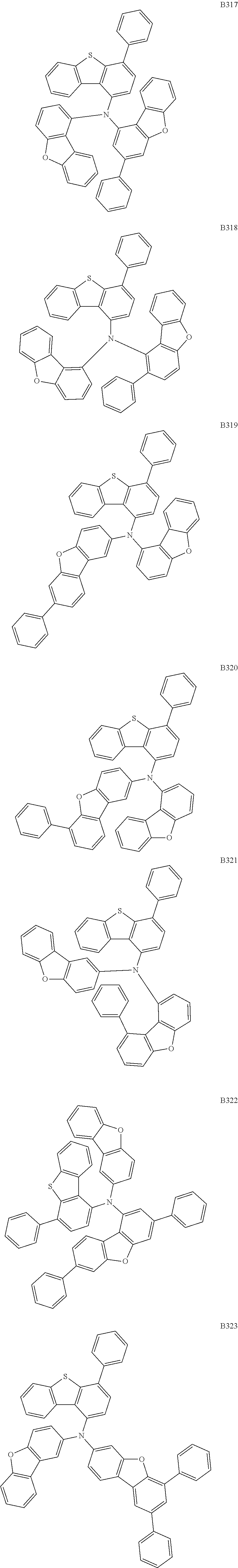

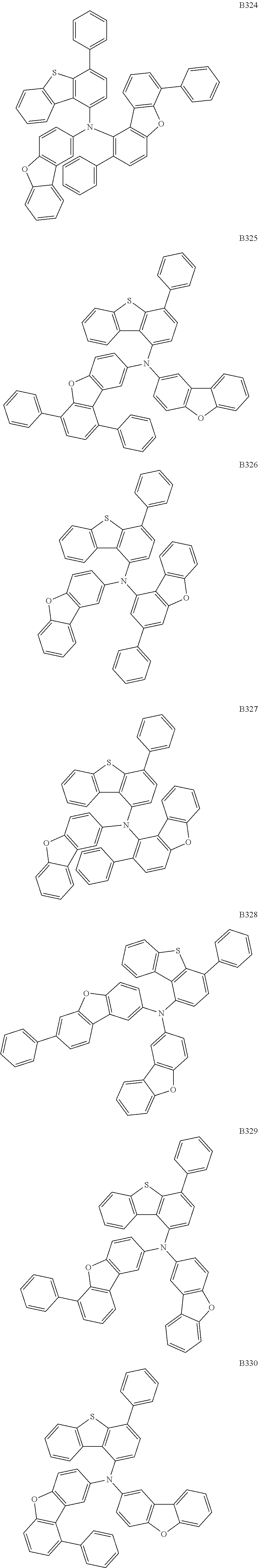

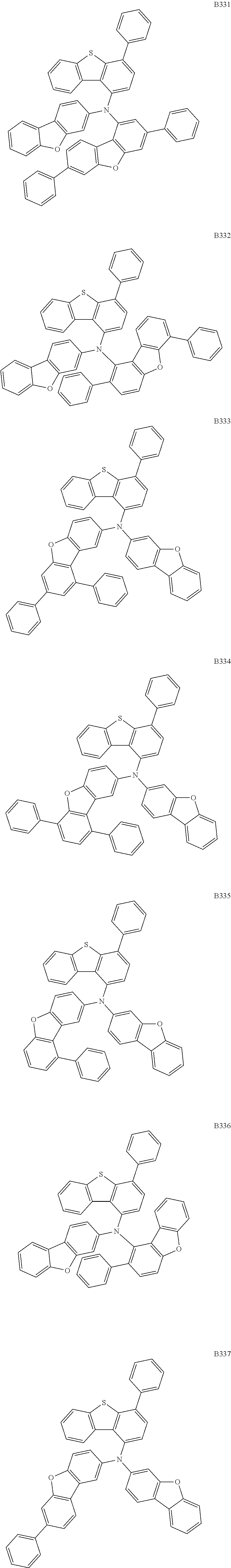

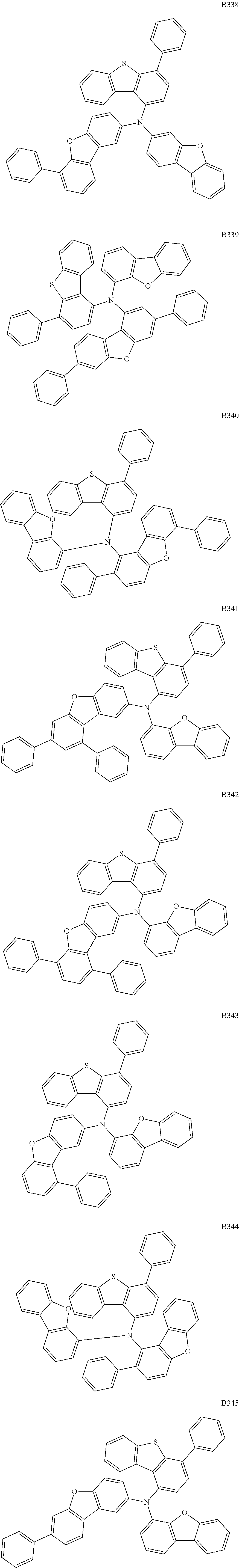

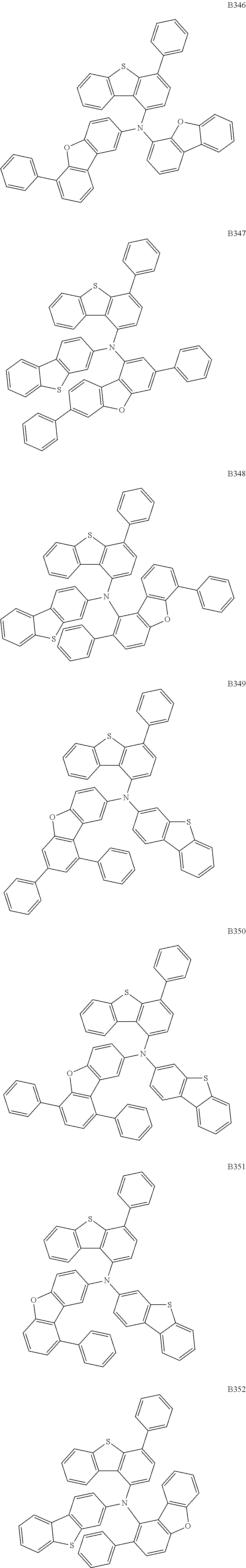

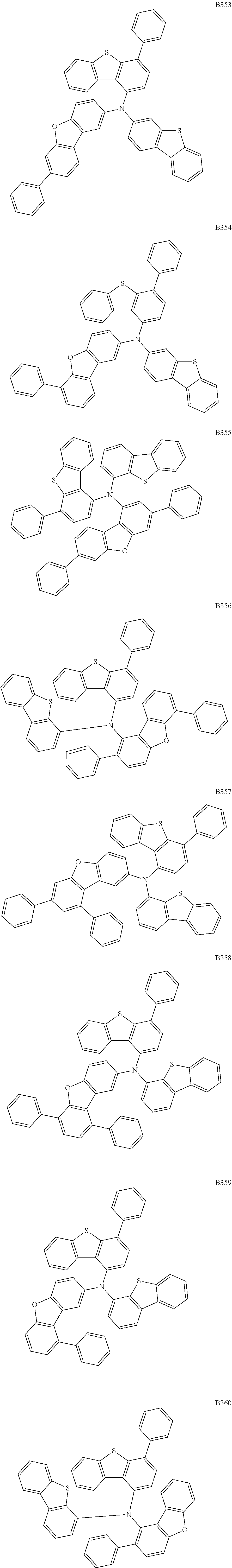

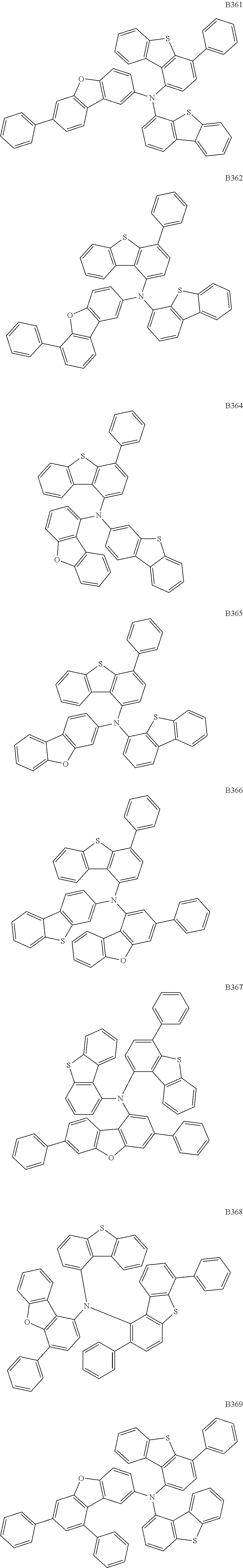

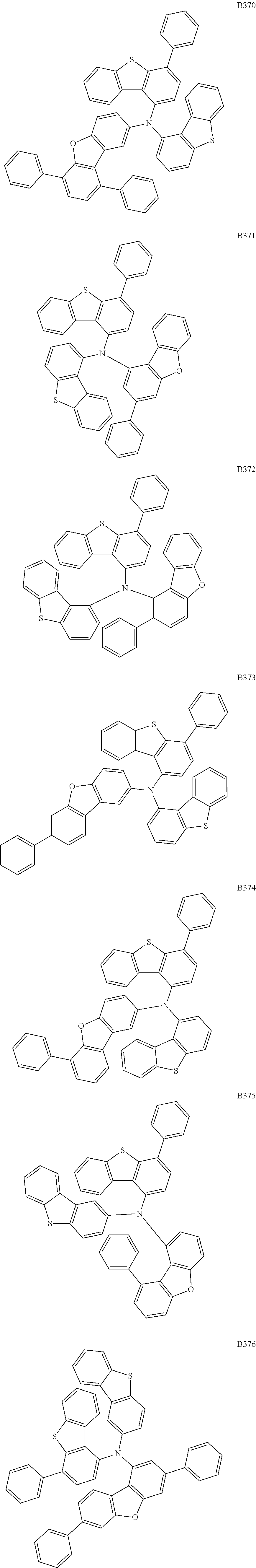

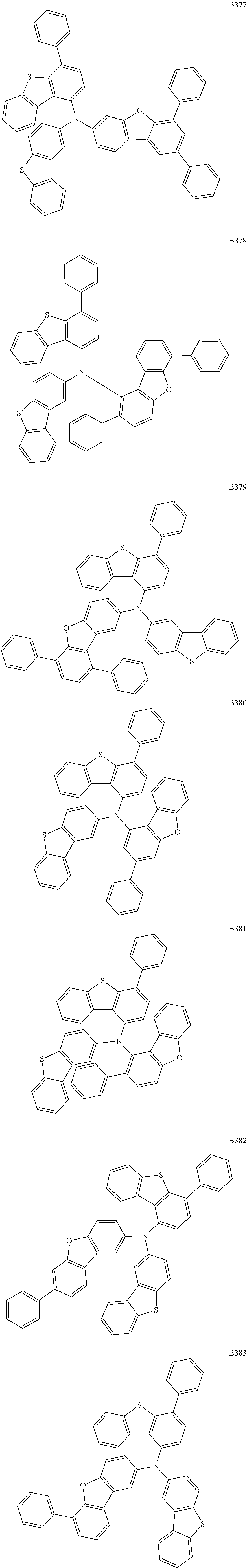

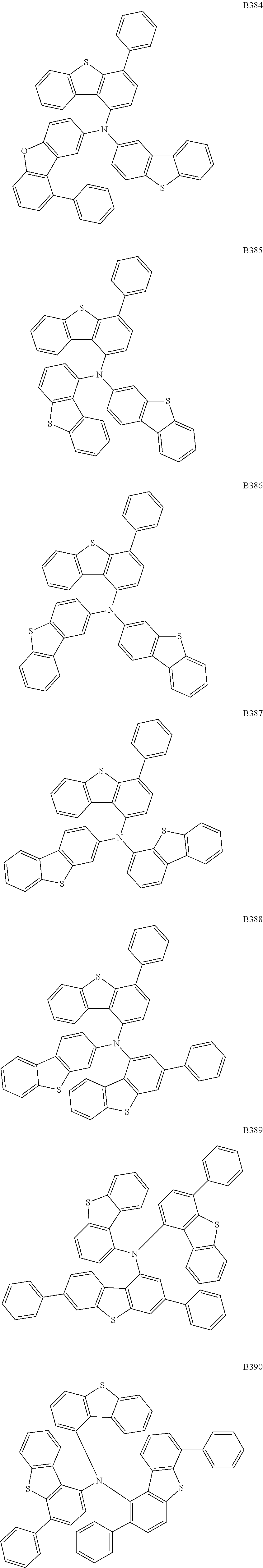

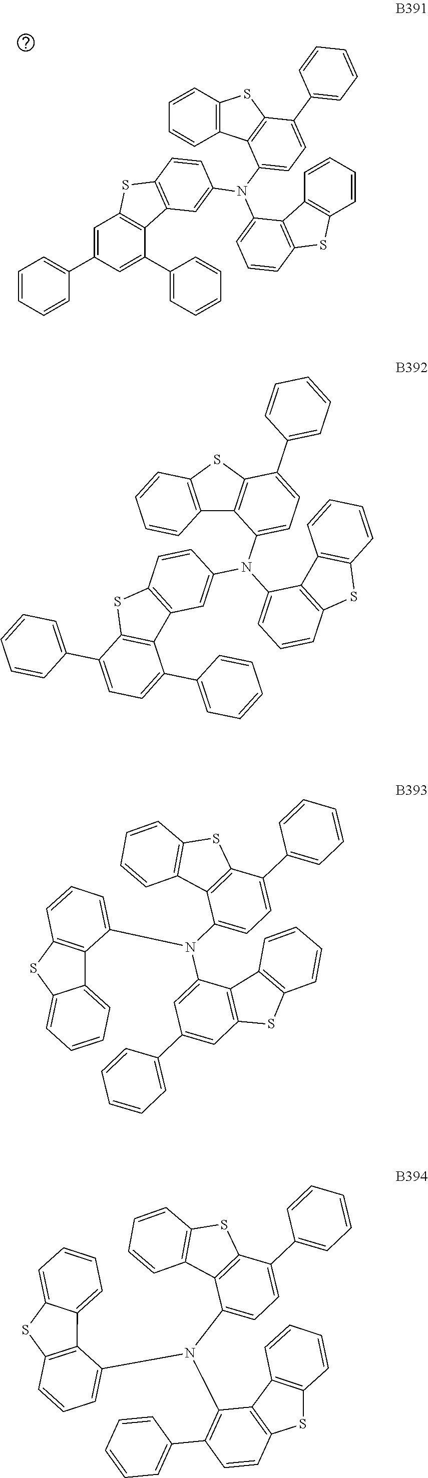 [SMILES: c1ccc(-c2cc(-c3ccccc3)c3c(c2)sc2ccc(N(c4cccc5sc6ccccc6c45)c4ccc(-c5ccccc5)c5sc6ccccc6c45)cc23)cc1 ; c1ccc(-c2ccc(-c3ccccc3)c3c2sc2ccc(N(c4cccc5sc6ccccc6c45)c4ccc(-c5ccccc5)c5sc6ccccc6c45)cc23)cc1 ; c1ccc(-c2cc(N(c3cccc4sc5ccccc5c34)c3ccc(-c4ccccc4)c4sc5ccccc5c34)c3c(c2)sc2ccccc23)cc1 ; c1ccc(-c2ccc3sc4ccccc4c3c2N(c2cccc3sc4ccccc4c23)c2ccc(-c3ccccc3)c3sc4ccccc4c23)cc1]

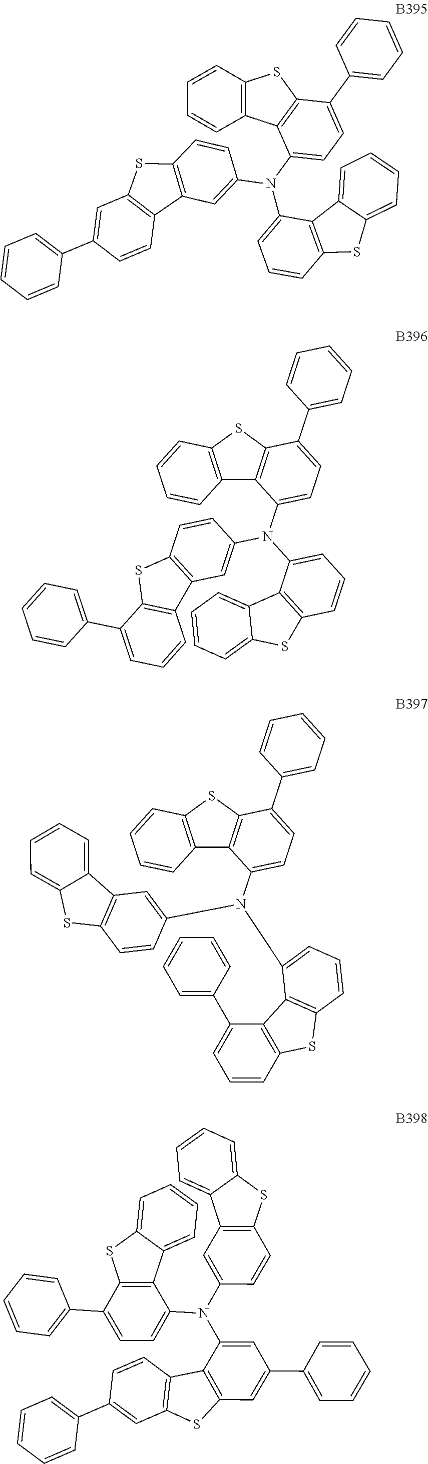 [SMILES: c1ccc(-c2ccc3c(c2)sc2ccc(N(c4cccc5sc6ccccc6c45)c4ccc(-c5ccccc5)c5sc6ccccc6c45)cc23)cc1 ; c1ccc(-c2cccc3c2sc2ccc(N(c4cccc5sc6ccccc6c45)c4ccc(-c5ccccc5)c5sc6ccccc6c45)cc23)cc1 ; c1ccc(-c2ccc(N(c3ccc4sc5ccccc5c4c3)c3cccc4sc5cccc(-c6ccccc6)c5c34)c3c2sc2ccccc23)cc1 ; c1ccc(-c2ccc3c(c2)sc2cc(-c4ccccc4)cc(N(c4ccc5sc6ccccc6c5c4)c4ccc(-c5ccccc5)c5sc6ccccc6c45)c23)cc1]

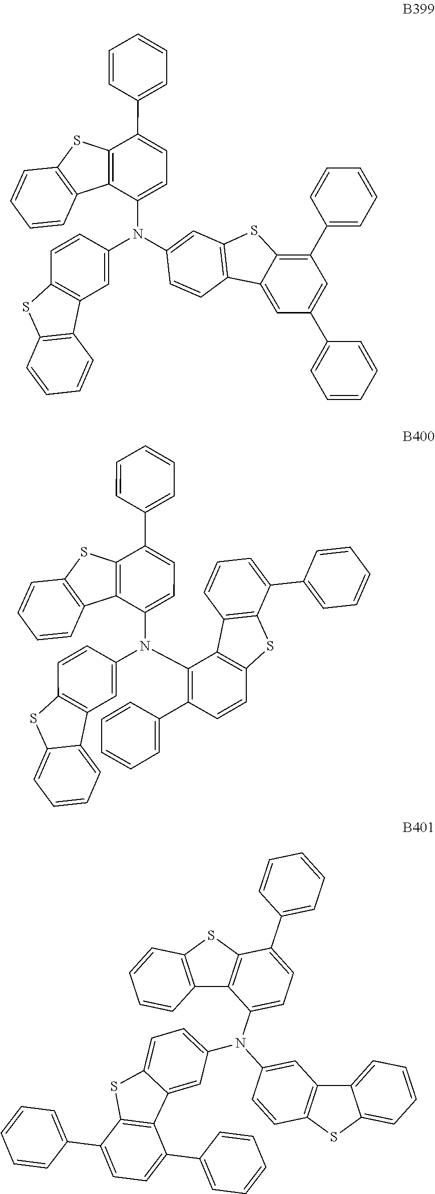 [SMILES: c1ccc(-c2cc(-c3ccccc3)c3sc4cc(N(c5ccc6sc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3sc4ccccc4c3c2)c2ccc(-c3ccccc3)c3sc4ccccc4c23)cc1 ; c1ccc(-c2ccc(-c3ccccc3)c3c2sc2ccc(N(c4ccc5sc6ccccc6c5c4)c4ccc(-c5ccccc5)c5sc6ccccc6c45)cc23)cc1]

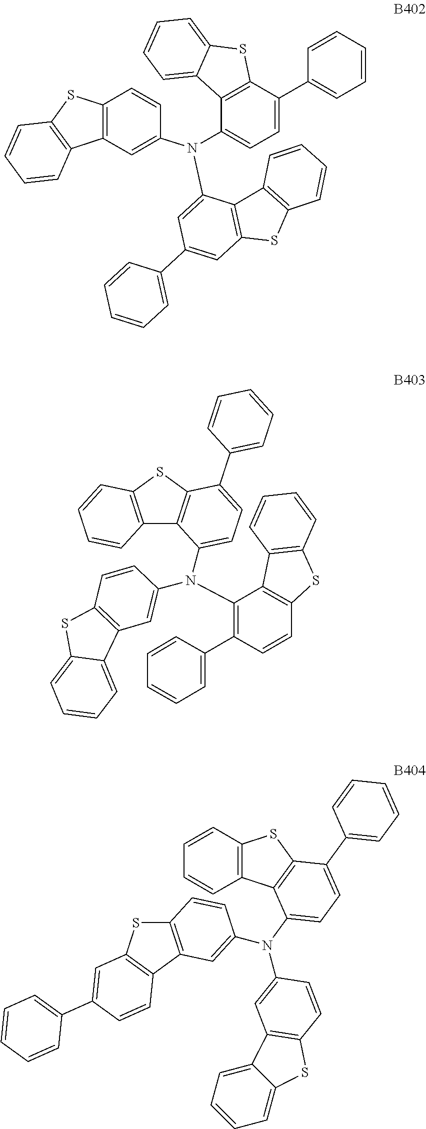 [SMILES: c1ccc(-c2cc(N(c3ccc4sc5ccccc5c4c3)c3ccc(-c4ccccc4)c4sc5ccccc5c34)c3c(c2)sc2ccccc23)cc1 ; c1ccc(-c2ccc3sc4ccccc4c3c2N(c2ccc3sc4ccccc4c3c2)c2ccc(-c3ccccc3)c3sc4ccccc4c23)cc1 ; c1ccc(-c2ccc3c(c2)sc2ccc(N(c4ccc5sc6ccccc6c5c4)c4ccc(-c5ccccc5)c5sc6ccccc6c45)cc23)cc1]

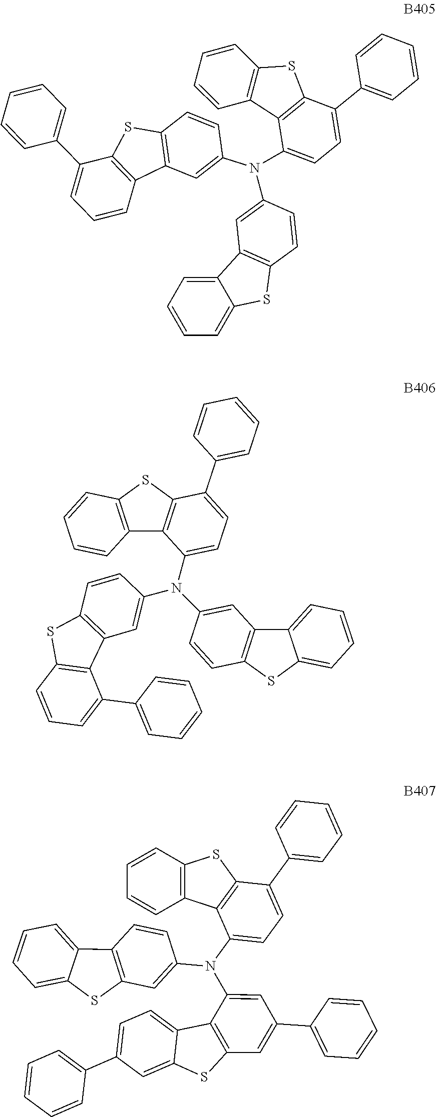 [SMILES: c1ccc(-c2cccc3c2sc2ccc(N(c4ccc5sc6ccccc6c5c4)c4ccc(-c5ccccc5)c5sc6ccccc6c45)cc23)cc1 ; c1ccc(-c2ccc(N(c3ccc4sc5ccccc5c4c3)c3ccc4sc5cccc(-c6ccccc6)c5c4c3)c3c2sc2ccccc23)cc1 ; c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1]

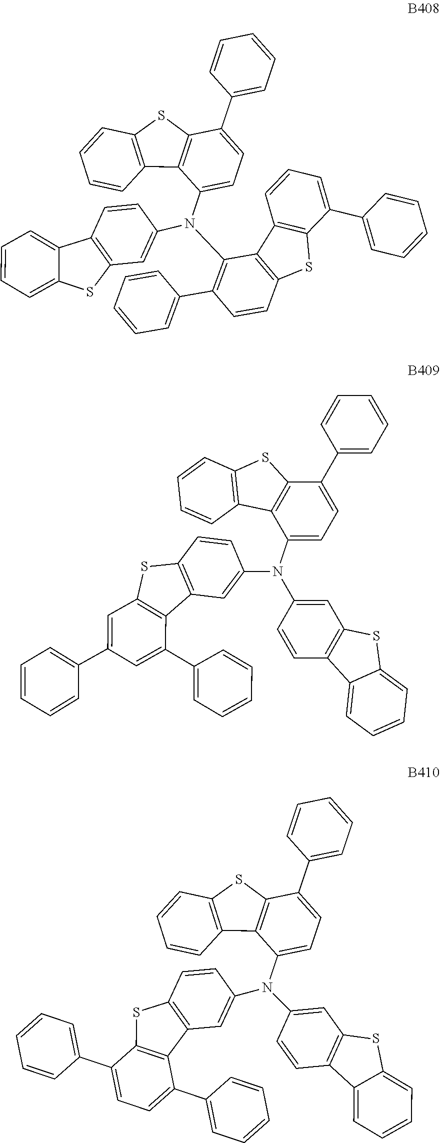 [SMILES: c1ccc(-c2ccc3sc4c(-c5ccccc5)cccc4c3c2N(c2ccc3c(c2)sc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1 ; c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1]

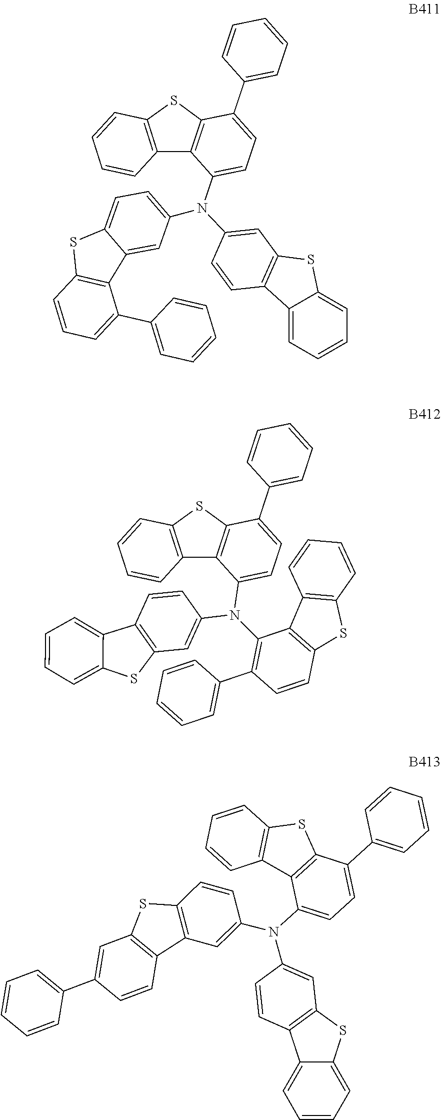 [SMILES: c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1 ; c1ccc(-c2ccc3c(c2)sc2ccc(N(c4ccc5c(c4)sc4ccccc45)c4ccc(-c5ccccc5)c5sc6ccccc6c45)cc23)cc1]

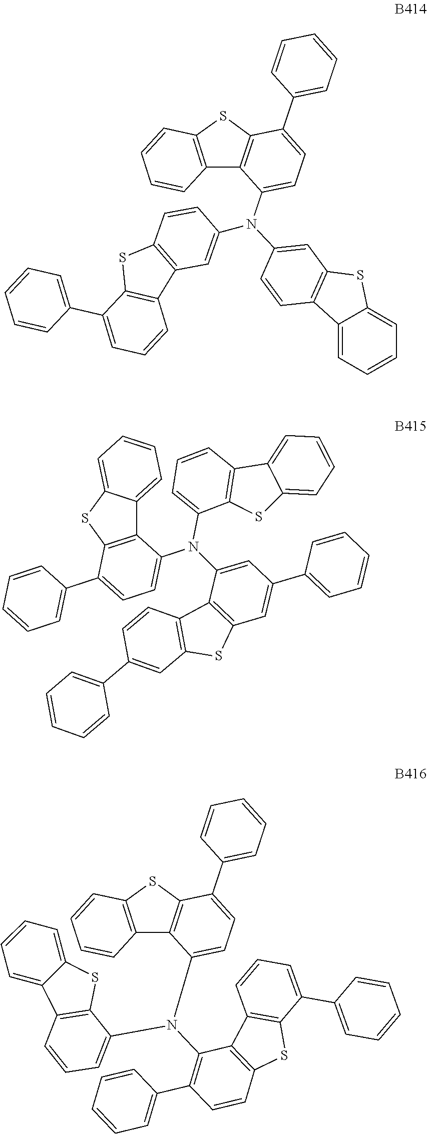 [SMILES: c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4ccc(-c5ccccc5)c5sc6ccccc6c45)c23)cc1 ; c1ccc(-c2ccc3sc4c(-c5ccccc5)cccc4c3c2N(c2cccc3c2sc2ccccc23)c2ccc(-c3ccccc3)c3sc4ccccc4c23)cc1]

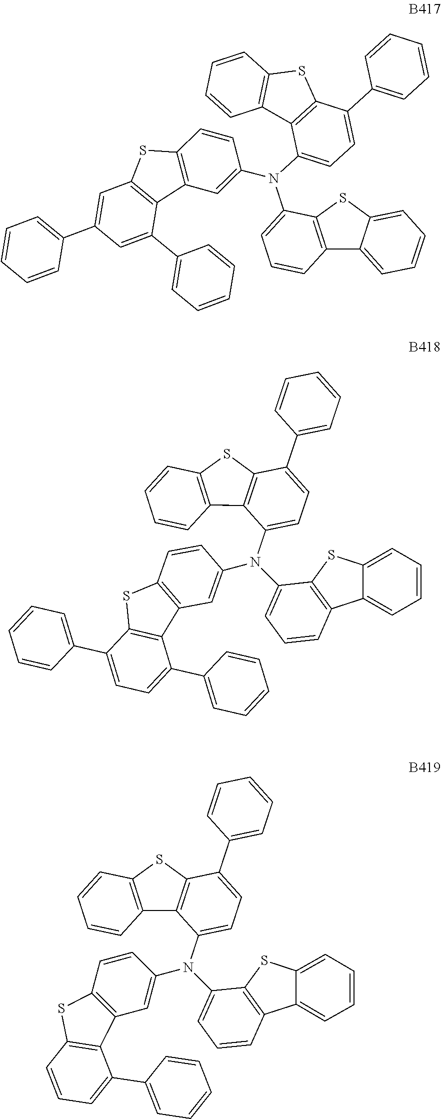 [SMILES: c1ccc(-c2cc(-c3ccccc3)c3c(c2)sc2ccc(N(c4cccc5c4sc4ccccc45)c4ccc(-c5ccccc5)c5sc6ccccc6c45)cc23)cc1 ; c1ccc(-c2ccc(-c3ccccc3)c3c2sc2ccc(N(c4cccc5c4sc4ccccc45)c4ccc(-c5ccccc5)c5sc6ccccc6c45)cc23)cc1 ; c1ccc(-c2ccc(N(c3ccc4sc5cccc(-c6ccccc6)c5c4c3)c3cccc4c3sc3ccccc34)c3c2sc2ccccc23)cc1]

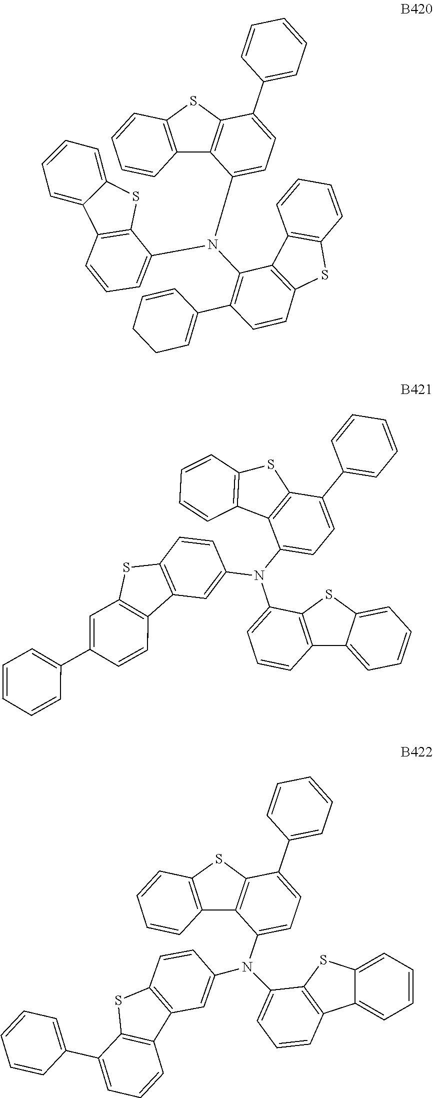 [SMILES: C1=CC(c2ccc3sc4ccccc4c3c2N(c2cccc3c2sc2ccccc23)c2ccc(-c3ccccc3)c3sc4ccccc4c23)=CCC1 ; c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2cccc3c2sc2ccc(N(c4cccc5c4sc4ccccc45)c4ccc(-c5ccccc5)c5sc6ccccc6c45)cc23)cc1]

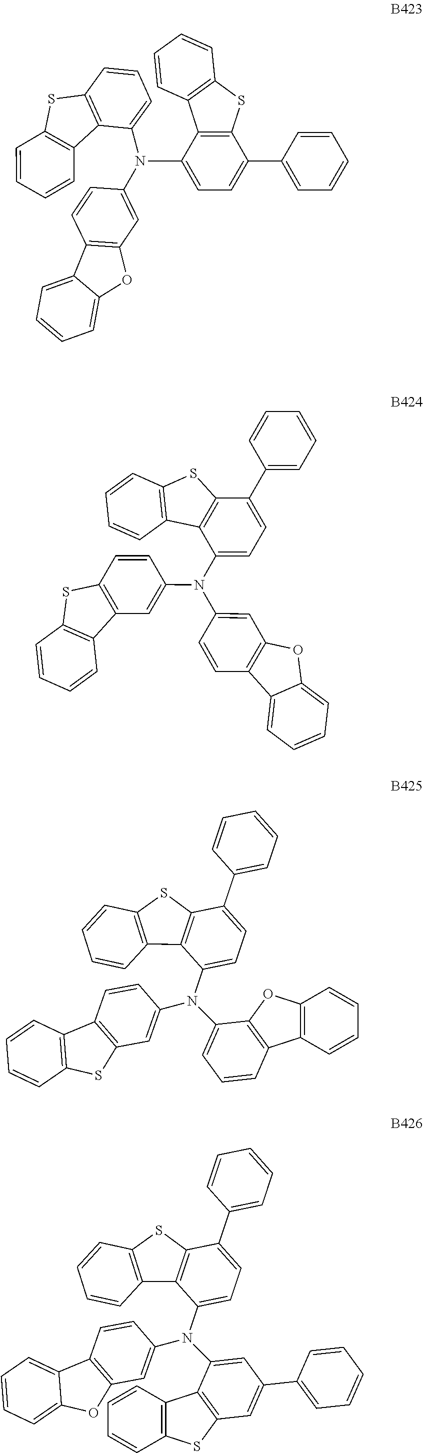 [SMILES: c1ccc(-c2ccc(N(c3ccc4c(c3)oc3ccccc34)c3cccc4sc5ccccc5c34)c3c2sc2ccccc23)cc1 ; c1ccc(-c2ccc(N(c3ccc4c(c3)oc3ccccc34)c3ccc4sc5ccccc5c4c3)c3c2sc2ccccc23)cc1 ; c1ccc(-c2ccc(N(c3ccc4c(c3)sc3ccccc34)c3cccc4c3oc3ccccc34)c3c2sc2ccccc23)cc1 ; c1ccc(-c2cc(N(c3ccc4c(c3)oc3ccccc34)c3ccc(-c4ccccc4)c4sc5ccccc5c34)c3c(c2)sc2ccccc23)cc1]

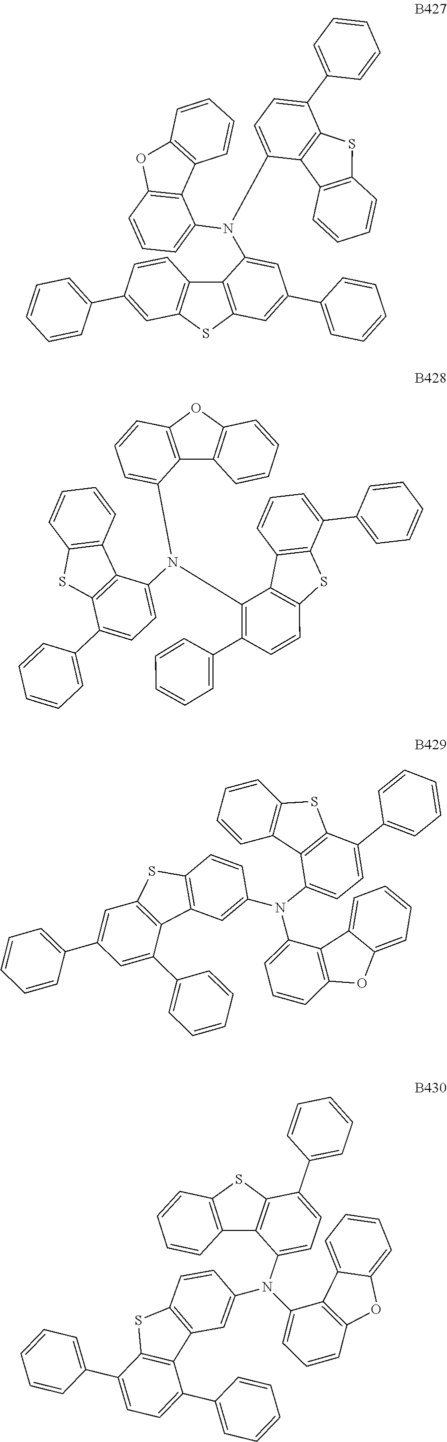 [SMILES: c1ccc(-c2ccc3c(c2)sc2cc(-c4ccccc4)cc(N(c4cccc5oc6ccccc6c45)c4ccc(-c5ccccc5)c5sc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3sc4ccccc4c23)cc1 ; c1ccc(-c2cc(-c3ccccc3)c3c(c2)sc2ccc(N(c4cccc5oc6ccccc6c45)c4ccc(-c5ccccc5)c5sc6ccccc6c45)cc23)cc1 ; c1ccc(-c2ccc(-c3ccccc3)c3c2sc2ccc(N(c4cccc5oc6ccccc6c45)c4ccc(-c5ccccc5)c5sc6ccccc6c45)cc23)cc1]

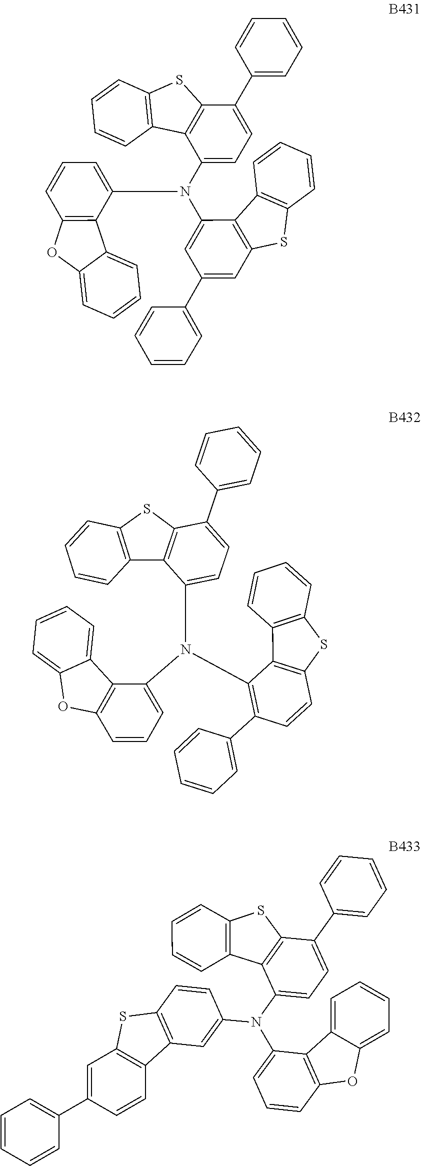 [SMILES: c1ccc(-c2cc(N(c3cccc4oc5ccccc5c34)c3ccc(-c4ccccc4)c4sc5ccccc5c34)c3c(c2)sc2ccccc23)cc1 ; c1ccc(-c2ccc3sc4ccccc4c3c2N(c2cccc3oc4ccccc4c23)c2ccc(-c3ccccc3)c3sc4ccccc4c23)cc1 ; c1ccc(-c2ccc3c(c2)sc2ccc(N(c4cccc5oc6ccccc6c45)c4ccc(-c5ccccc5)c5sc6ccccc6c45)cc23)cc1]

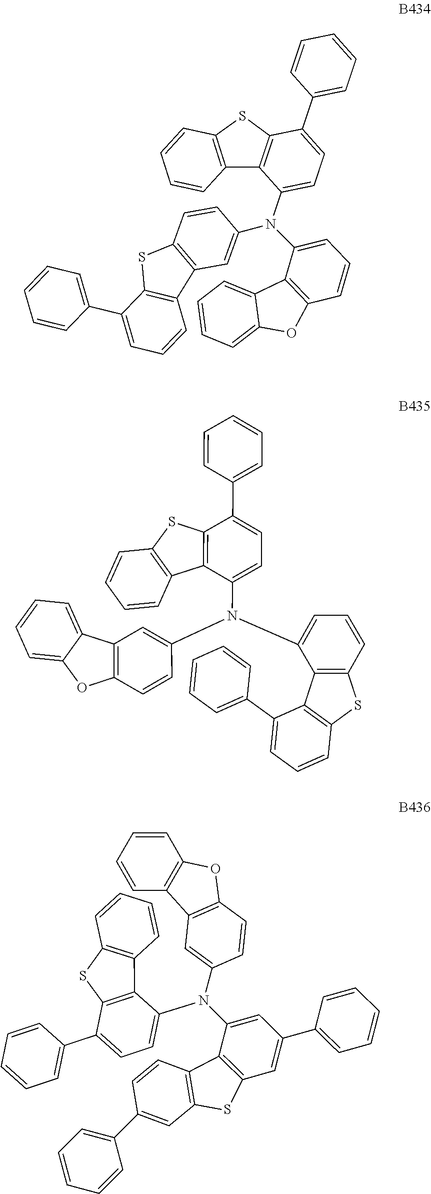 [SMILES: c1ccc(-c2cccc3c2sc2ccc(N(c4cccc5oc6ccccc6c45)c4ccc(-c5ccccc5)c5sc6ccccc6c45)cc23)cc1 ; c1ccc(-c2ccc(N(c3ccc4oc5ccccc5c4c3)c3cccc4sc5cccc(-c6ccccc6)c5c34)c3c2sc2ccccc23)cc1 ; c1ccc(-c2ccc3c(c2)sc2cc(-c4ccccc4)cc(N(c4ccc5oc6ccccc6c5c4)c4ccc(-c5ccccc5)c5sc6ccccc6c45)c23)cc1]

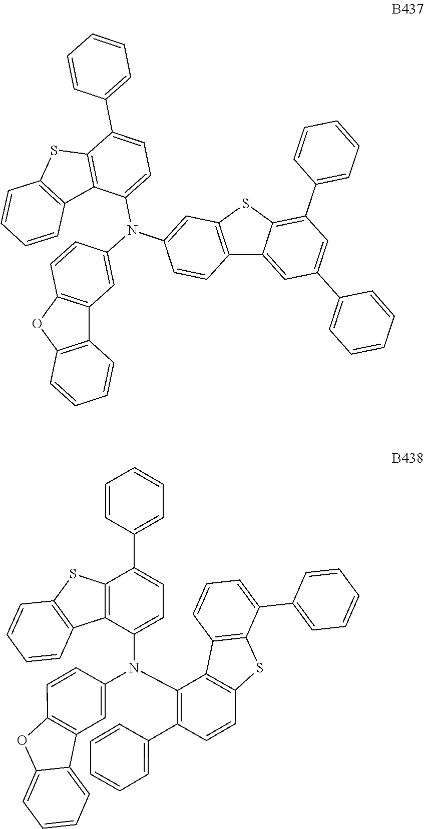 [SMILES: c1ccc(-c2cc(-c3ccccc3)c3sc4cc(N(c5ccc6oc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3oc4ccccc4c3c2)c2ccc(-c3ccccc3)c3sc4ccccc4c23)cc1]

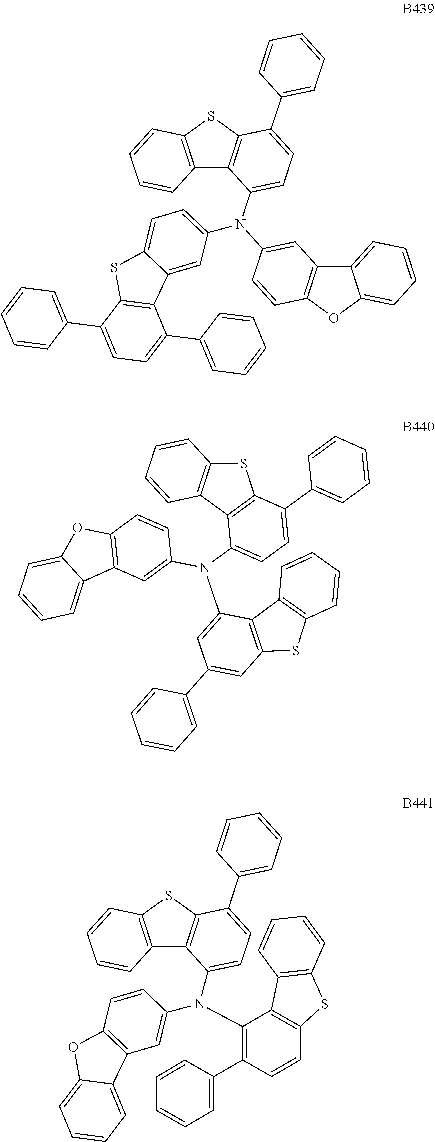 [SMILES: c1ccc(-c2ccc(-c3ccccc3)c3c2sc2ccc(N(c4ccc5oc6ccccc6c5c4)c4ccc(-c5ccccc5)c5sc6ccccc6c45)cc23)cc1 ; c1ccc(-c2cc(N(c3ccc4oc5ccccc5c4c3)c3ccc(-c4ccccc4)c4sc5ccccc5c34)c3c(c2)sc2ccccc23)cc1 ; c1ccc(-c2ccc3sc4ccccc4c3c2N(c2ccc3oc4ccccc4c3c2)c2ccc(-c3ccccc3)c3sc4ccccc4c23)cc1]

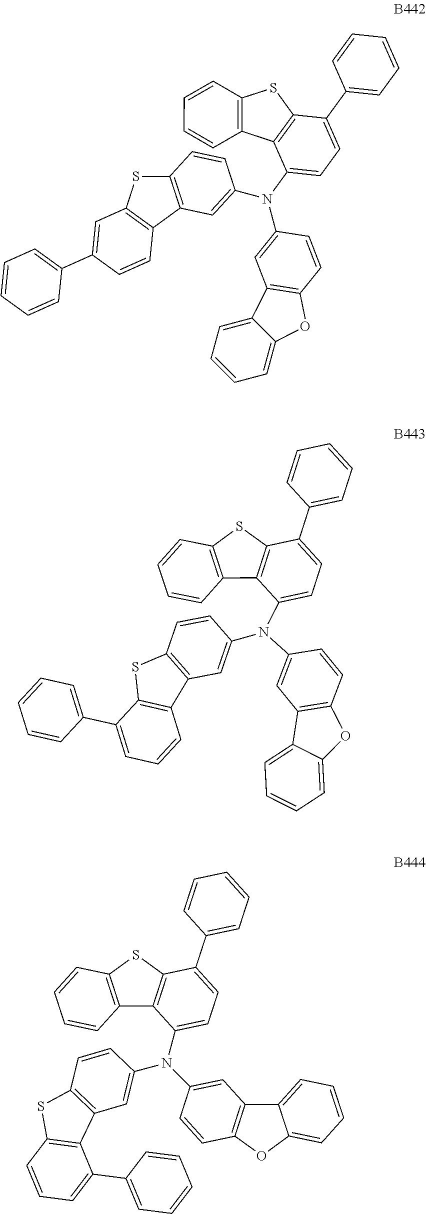 [SMILES: c1ccc(-c2ccc3c(c2)sc2ccc(N(c4ccc5oc6ccccc6c5c4)c4ccc(-c5ccccc5)c5sc6ccccc6c45)cc23)cc1 ; c1ccc(-c2cccc3c2sc2ccc(N(c4ccc5oc6ccccc6c5c4)c4ccc(-c5ccccc5)c5sc6ccccc6c45)cc23)cc1 ; c1ccc(-c2ccc(N(c3ccc4oc5ccccc5c4c3)c3ccc4sc5cccc(-c6ccccc6)c5c4c3)c3c2sc2ccccc23)cc1]

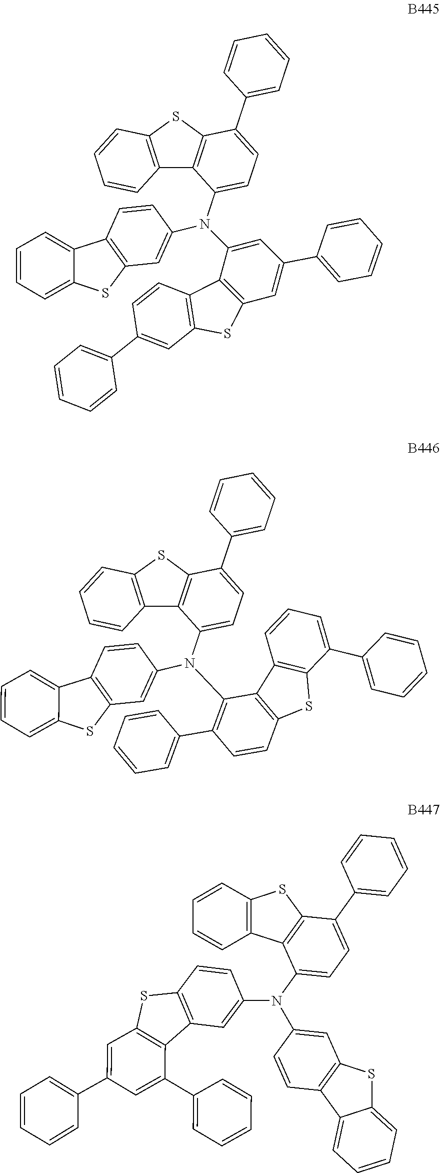 [SMILES: c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1]

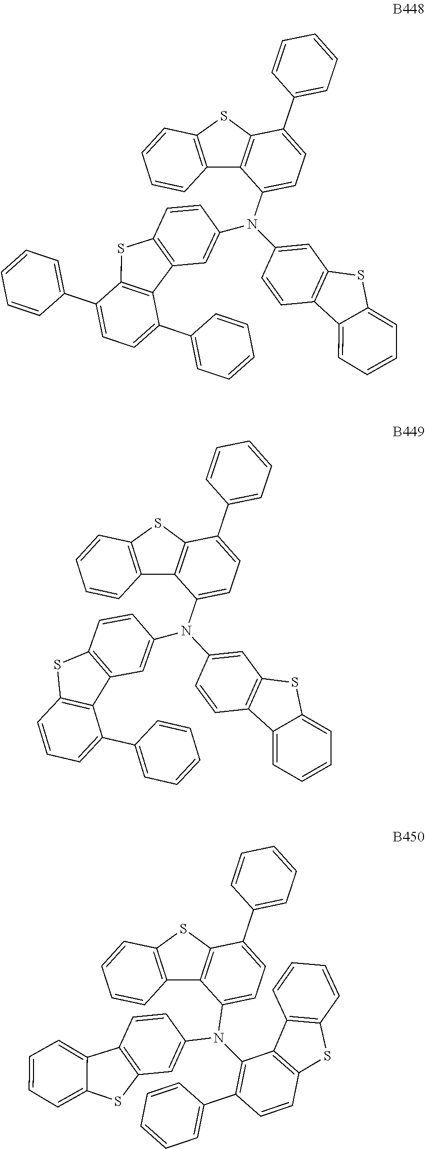 [SMILES: c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1 ; c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1]

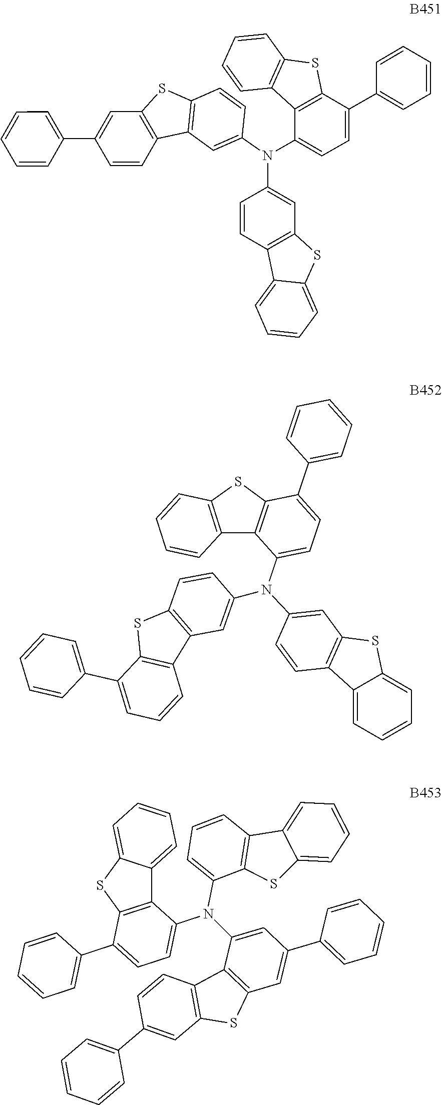 [SMILES: c1ccc(-c2ccc3c(c2)sc2ccc(N(c4ccc5c(c4)sc4ccccc45)c4ccc(-c5ccccc5)c5sc6ccccc6c45)cc23)cc1 ; c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4ccc(-c5ccccc5)c5sc6ccccc6c45)c23)cc1]

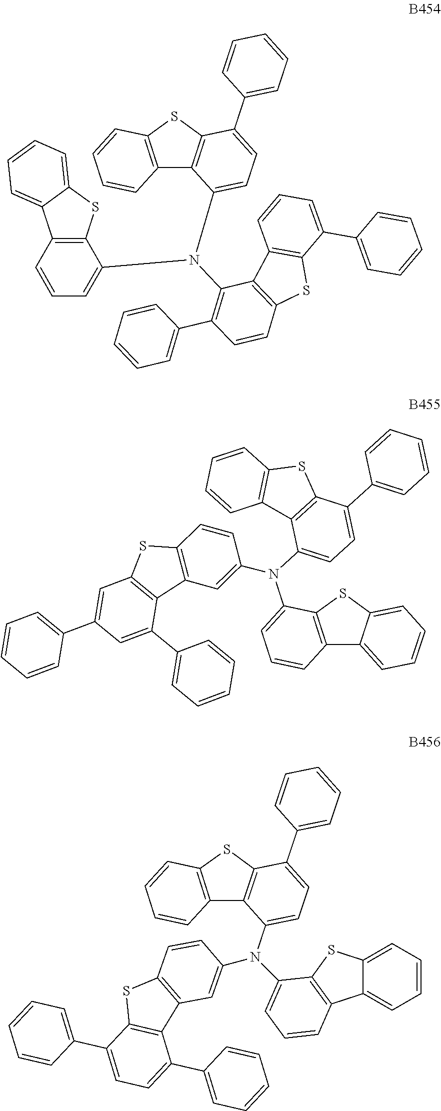 [SMILES: c1ccc(-c2ccc3sc4c(-c5ccccc5)cccc4c3c2N(c2cccc3c2sc2ccccc23)c2ccc(-c3ccccc3)c3sc4ccccc4c23)cc1 ; c1ccc(-c2cc(-c3ccccc3)c3c(c2)sc2ccc(N(c4cccc5c4sc4ccccc45)c4ccc(-c5ccccc5)c5sc6ccccc6c45)cc23)cc1 ; c1ccc(-c2ccc(-c3ccccc3)c3c2sc2ccc(N(c4cccc5c4sc4ccccc45)c4ccc(-c5ccccc5)c5sc6ccccc6c45)cc23)cc1]

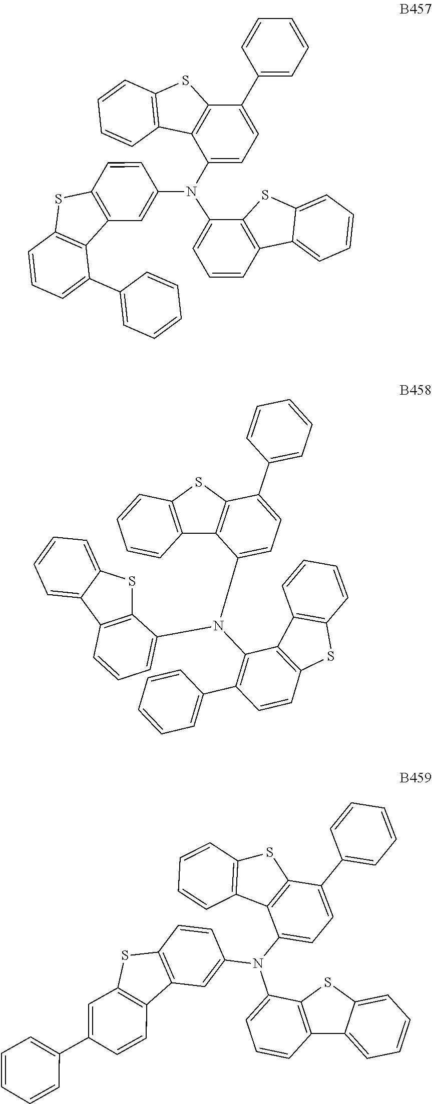 [SMILES: c1ccc(-c2ccc(N(c3ccc4sc5cccc(-c6ccccc6)c5c4c3)c3cccc4c3sc3ccccc34)c3c2sc2ccccc23)cc1 ; c1ccc(-c2ccc3sc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4sc4ccccc45)c4ccc(-c5ccccc5)c5sc6ccccc6c45)cc23)cc1]

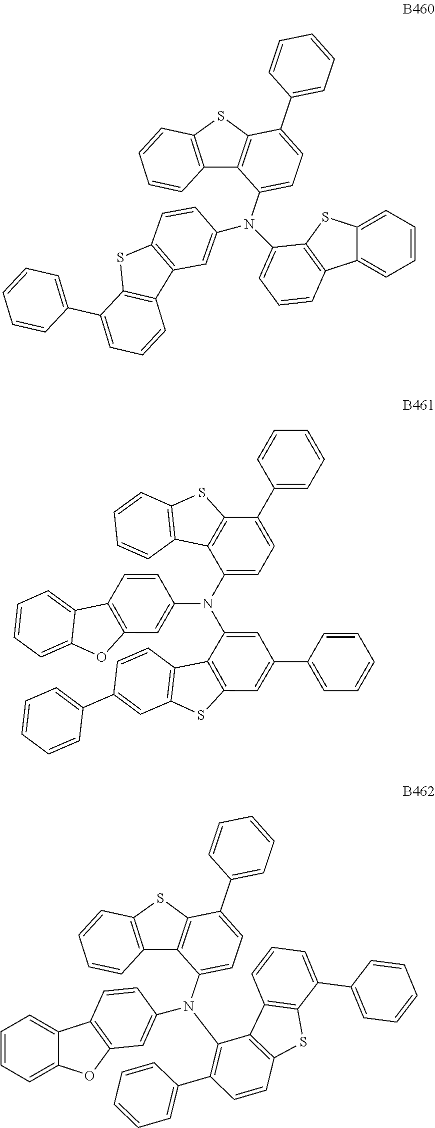 [SMILES: c1ccc(-c2cccc3c2sc2ccc(N(c4cccc5c4sc4ccccc45)c4ccc(-c5ccccc5)c5sc6ccccc6c45)cc23)cc1 ; c1ccc(-c2ccc3c(c2)sc2cc(-c4ccccc4)cc(N(c4ccc5c(c4)oc4ccccc45)c4ccc(-c5ccccc5)c5sc6ccccc6c45)c23)cc1 ; c1ccc(-c2ccc3sc4c(-c5ccccc5)cccc4c3c2N(c2ccc3c(c2)oc2ccccc23)c2ccc(-c3ccccc3)c3sc4ccccc4c23)cc1]

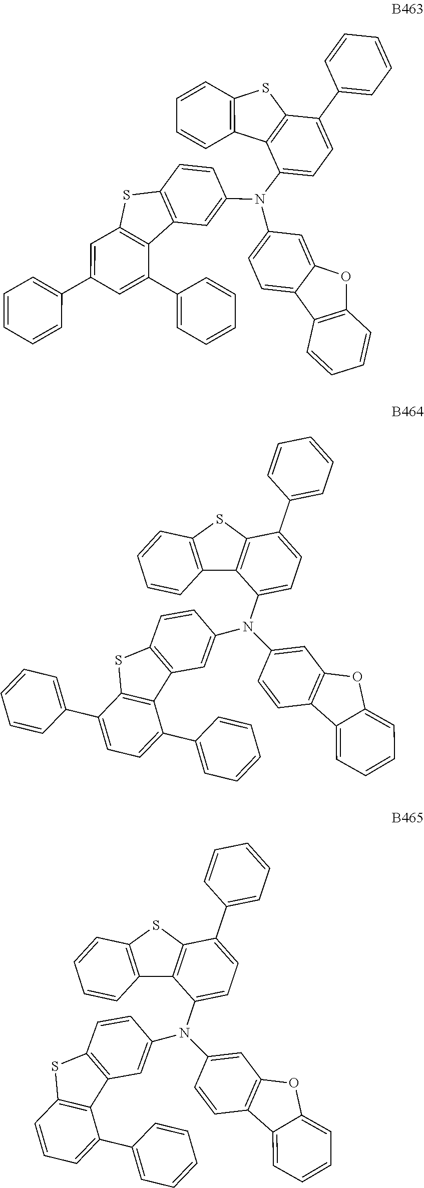 [SMILES: c1ccc(-c2cc(-c3ccccc3)c3c(c2)sc2ccc(N(c4ccc5c(c4)oc4ccccc45)c4ccc(-c5ccccc5)c5sc6ccccc6c45)cc23)cc1 ; c1ccc(-c2ccc(-c3ccccc3)c3c2sc2ccc(N(c4ccc5c(c4)oc4ccccc45)c4ccc(-c5ccccc5)c5sc6ccccc6c45)cc23)cc1 ; c1ccc(-c2ccc(N(c3ccc4c(c3)oc3ccccc34)c3ccc4sc5cccc(-c6ccccc6)c5c4c3)c3c2sc2ccccc23)cc1]

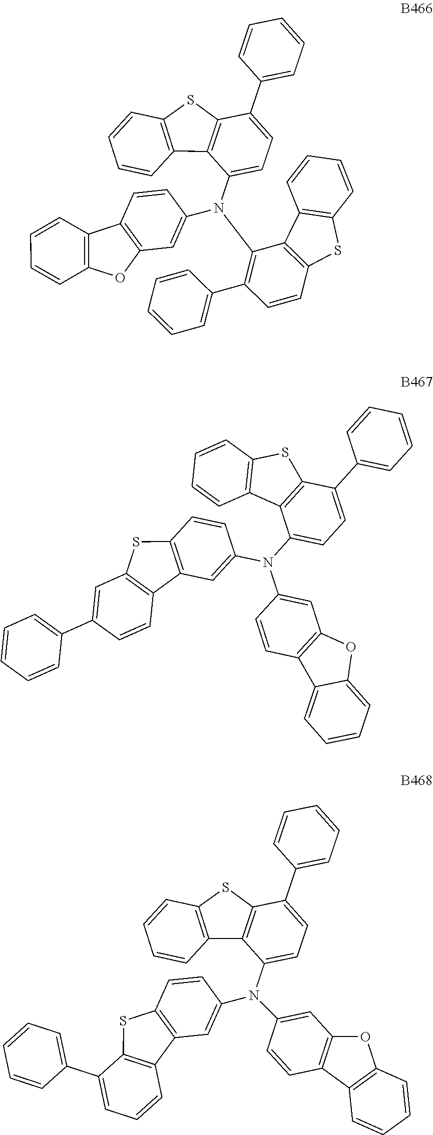 [SMILES: c1ccc(-c2ccc3sc4ccccc4c3c2N(c2ccc3c(c2)oc2ccccc23)c2ccc(-c3ccccc3)c3sc4ccccc4c23)cc1 ; c1ccc(-c2ccc3c(c2)sc2ccc(N(c4ccc5c(c4)oc4ccccc45)c4ccc(-c5ccccc5)c5sc6ccccc6c45)cc23)cc1 ; c1ccc(-c2cccc3c2sc2ccc(N(c4ccc5c(c4)oc4ccccc45)c4ccc(-c5ccccc5)c5sc6ccccc6c45)cc23)cc1]

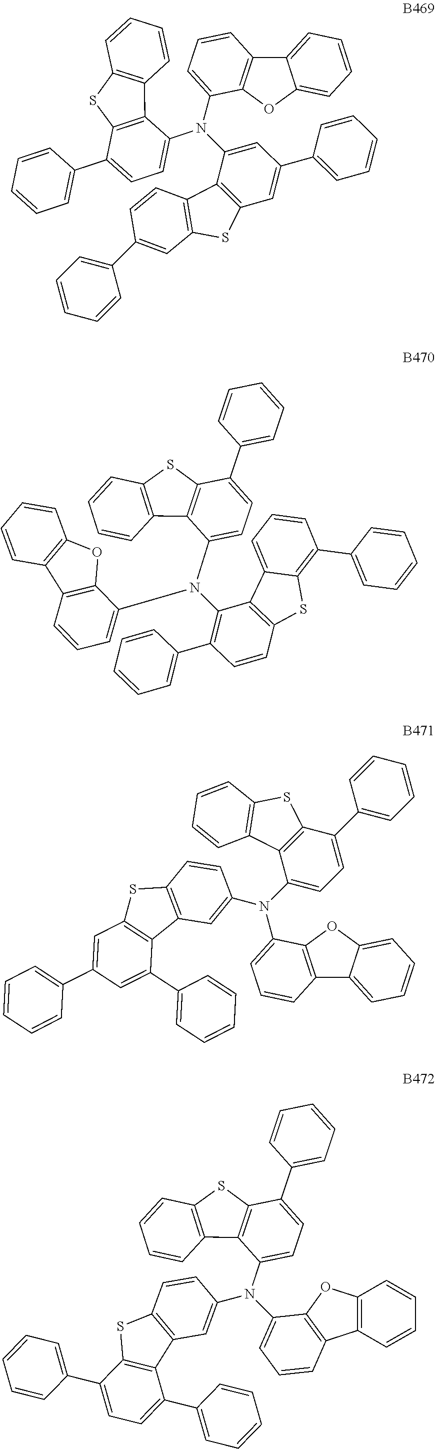 [SMILES: c1ccc(-c2ccc3c(c2)sc2cc(-c4ccccc4)cc(N(c4cccc5c4oc4ccccc45)c4ccc(-c5ccccc5)c5sc6ccccc6c45)c23)cc1 ; c1ccc(-c2ccc3sc4c(-c5ccccc5)cccc4c3c2N(c2cccc3c2oc2ccccc23)c2ccc(-c3ccccc3)c3sc4ccccc4c23)cc1 ; c1ccc(-c2cc(-c3ccccc3)c3c(c2)sc2ccc(N(c4cccc5c4oc4ccccc45)c4ccc(-c5ccccc5)c5sc6ccccc6c45)cc23)cc1 ; c1ccc(-c2ccc(-c3ccccc3)c3c2sc2ccc(N(c4cccc5c4oc4ccccc45)c4ccc(-c5ccccc5)c5sc6ccccc6c45)cc23)cc1]

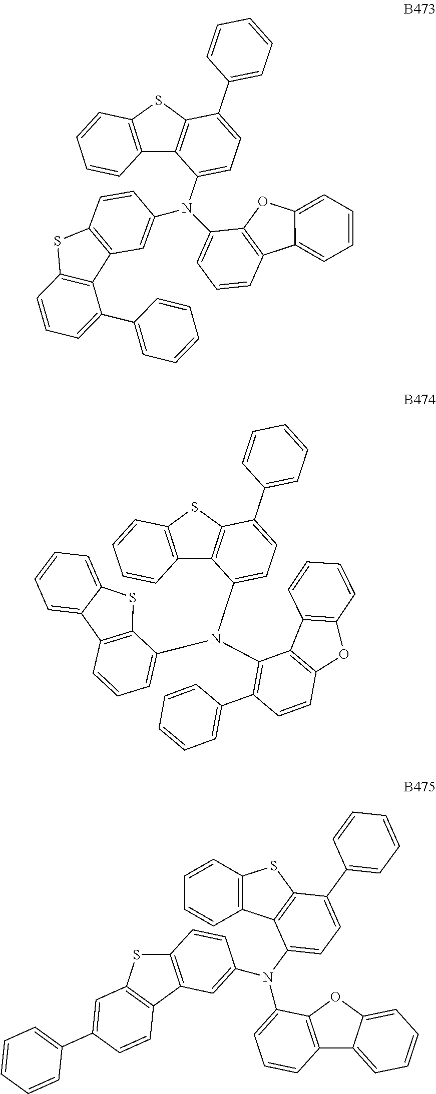 [SMILES: c1ccc(-c2ccc(N(c3ccc4sc5cccc(-c6ccccc6)c5c4c3)c3cccc4c3oc3ccccc34)c3c2sc2ccccc23)cc1 ; c1ccc(-c2ccc3oc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4oc4ccccc45)c4ccc(-c5ccccc5)c5sc6ccccc6c45)cc23)cc1]

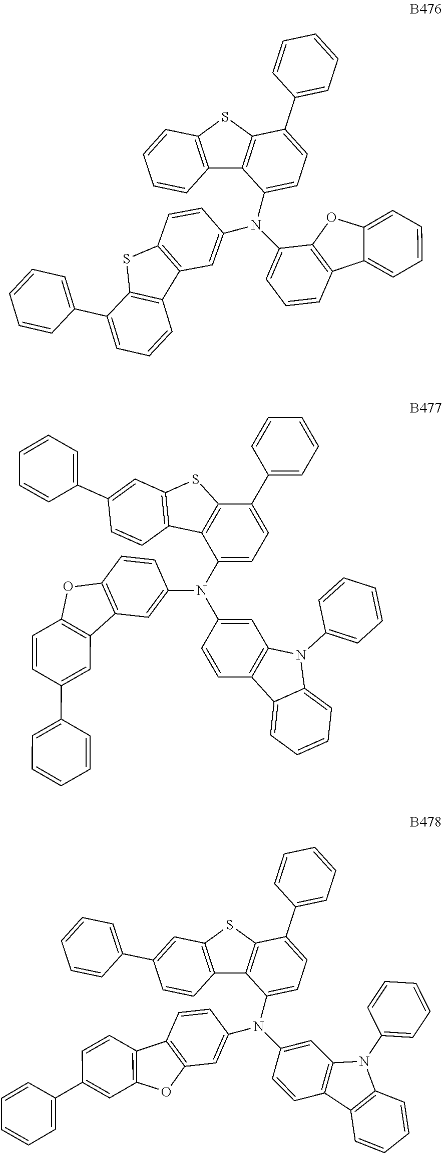 [SMILES: c1ccc(-c2cccc3c2sc2ccc(N(c4cccc5c4oc4ccccc45)c4ccc(-c5ccccc5)c5sc6ccccc6c45)cc23)cc1 ; c1ccc(-c2ccc3c(c2)sc2c(-c4ccccc4)ccc(N(c4ccc5oc6ccc(-c7ccccc7)cc6c5c4)c4ccc5c6ccccc6n(-c6ccccc6)c5c4)c23)cc1 ; c1ccc(-c2ccc3c(c2)oc2cc(N(c4ccc5c6ccccc6n(-c6ccccc6)c5c4)c4ccc(-c5ccccc5)c5sc6cc(-c7ccccc7)ccc6c45)ccc23)cc1]

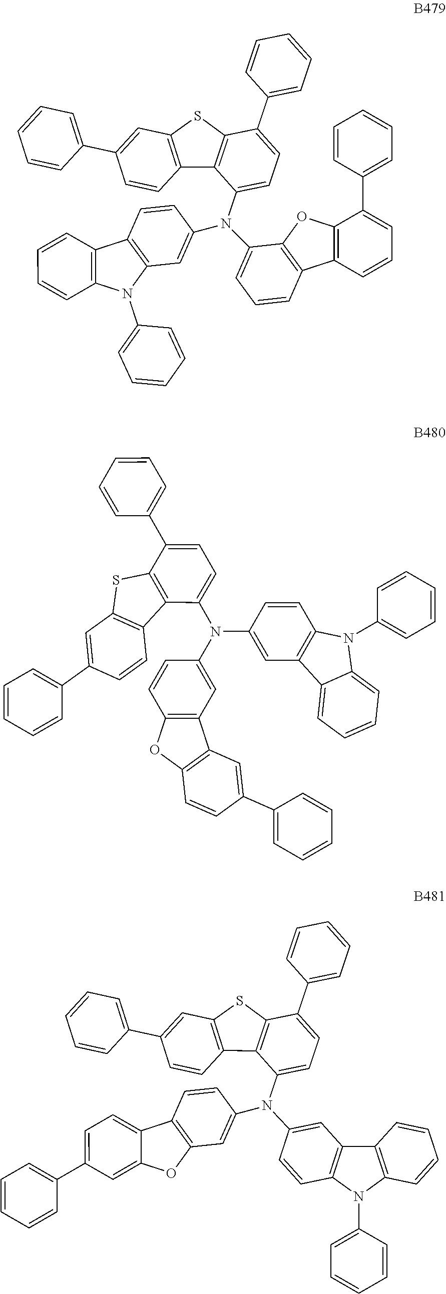 [SMILES: c1ccc(-c2ccc3c(c2)sc2c(-c4ccccc4)ccc(N(c4ccc5c6ccccc6n(-c6ccccc6)c5c4)c4cccc5c4oc4c(-c6ccccc6)cccc45)c23)cc1 ; c1ccc(-c2ccc3c(c2)sc2c(-c4ccccc4)ccc(N(c4ccc5oc6ccc(-c7ccccc7)cc6c5c4)c4ccc5c(c4)c4ccccc4n5-c4ccccc4)c23)cc1 ; c1ccc(-c2ccc3c(c2)oc2cc(N(c4ccc5c(c4)c4ccccc4n5-c4ccccc4)c4ccc(-c5ccccc5)c5sc6cc(-c7ccccc7)ccc6c45)ccc23)cc1]

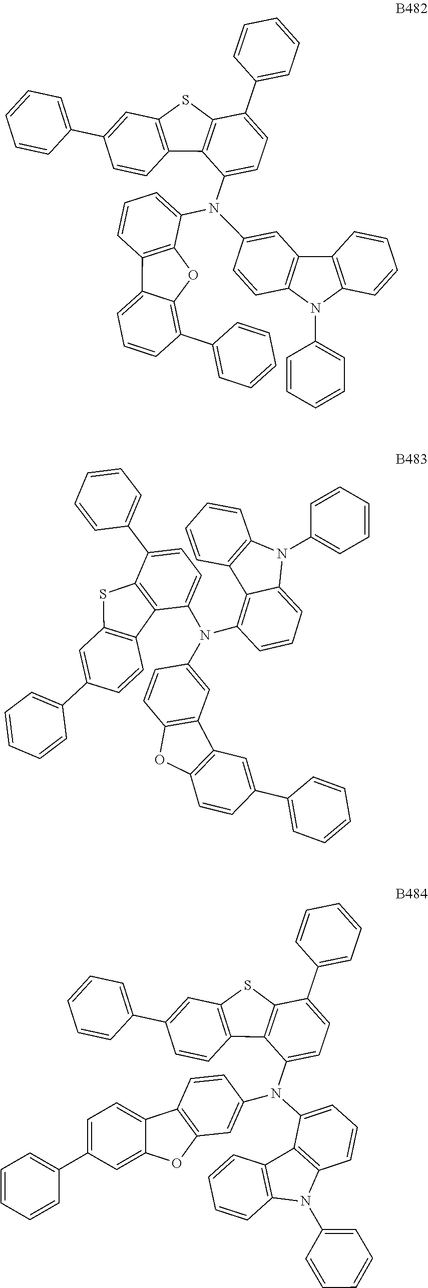 [SMILES: c1ccc(-c2ccc3c(c2)sc2c(-c4ccccc4)ccc(N(c4ccc5c(c4)c4ccccc4n5-c4ccccc4)c4cccc5c4oc4c(-c6ccccc6)cccc45)c23)cc1 ; c1ccc(-c2ccc3c(c2)sc2c(-c4ccccc4)ccc(N(c4ccc5oc6ccc(-c7ccccc7)cc6c5c4)c4cccc5c4c4ccccc4n5-c4ccccc4)c23)cc1 ; c1ccc(-c2ccc3c(c2)oc2cc(N(c4ccc(-c5ccccc5)c5sc6cc(-c7ccccc7)ccc6c45)c4cccc5c4c4ccccc4n5-c4ccccc4)ccc23)cc1]

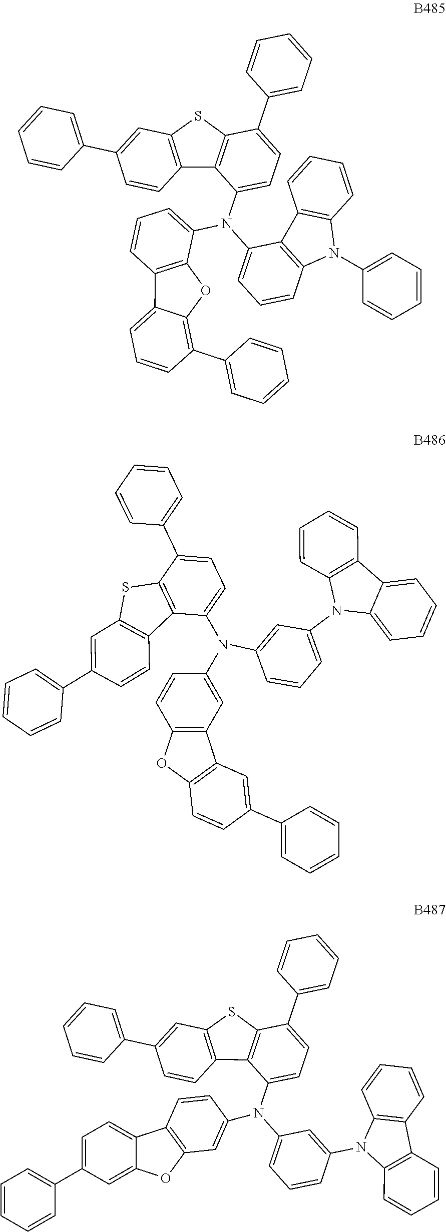 [SMILES: c1ccc(-c2ccc3c(c2)sc2c(-c4ccccc4)ccc(N(c4cccc5c4oc4c(-c6ccccc6)cccc45)c4cccc5c4c4ccccc4n5-c4ccccc4)c23)cc1 ; c1ccc(-c2ccc3c(c2)sc2c(-c4ccccc4)ccc(N(c4cccc(-n5c6ccccc6c6ccccc65)c4)c4ccc5oc6ccc(-c7ccccc7)cc6c5c4)c23)cc1 ; c1ccc(-c2ccc3c(c2)oc2cc(N(c4cccc(-n5c6ccccc6c6ccccc65)c4)c4ccc(-c5ccccc5)c5sc6cc(-c7ccccc7)ccc6c45)ccc23)cc1]

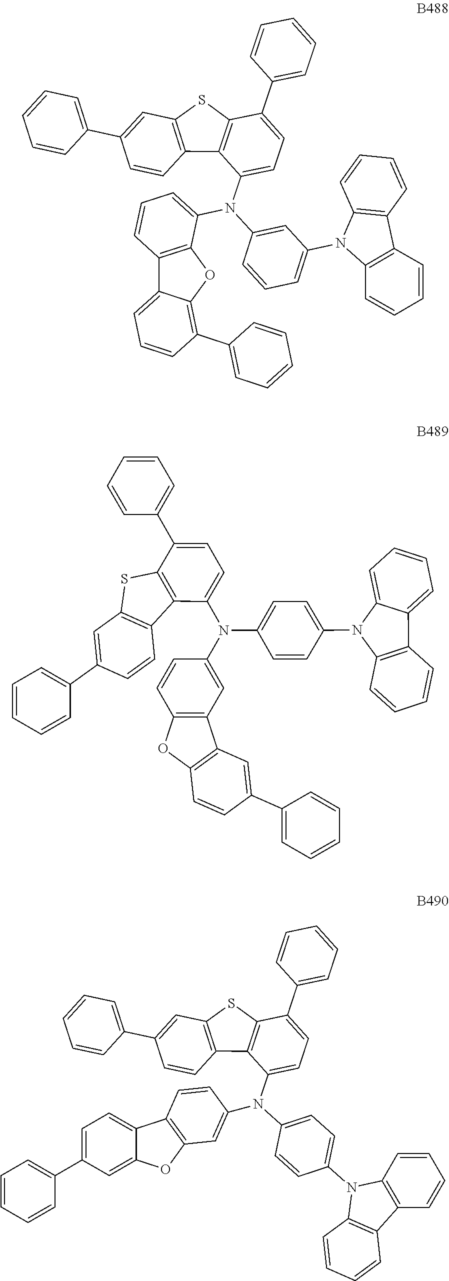 [SMILES: c1ccc(-c2ccc3c(c2)sc2c(-c4ccccc4)ccc(N(c4cccc(-n5c6ccccc6c6ccccc65)c4)c4cccc5c4oc4c(-c6ccccc6)cccc45)c23)cc1 ; c1ccc(-c2ccc3c(c2)sc2c(-c4ccccc4)ccc(N(c4ccc(-n5c6ccccc6c6ccccc65)cc4)c4ccc5oc6ccc(-c7ccccc7)cc6c5c4)c23)cc1 ; c1ccc(-c2ccc3c(c2)oc2cc(N(c4ccc(-n5c6ccccc6c6ccccc65)cc4)c4ccc(-c5ccccc5)c5sc6cc(-c7ccccc7)ccc6c45)ccc23)cc1]

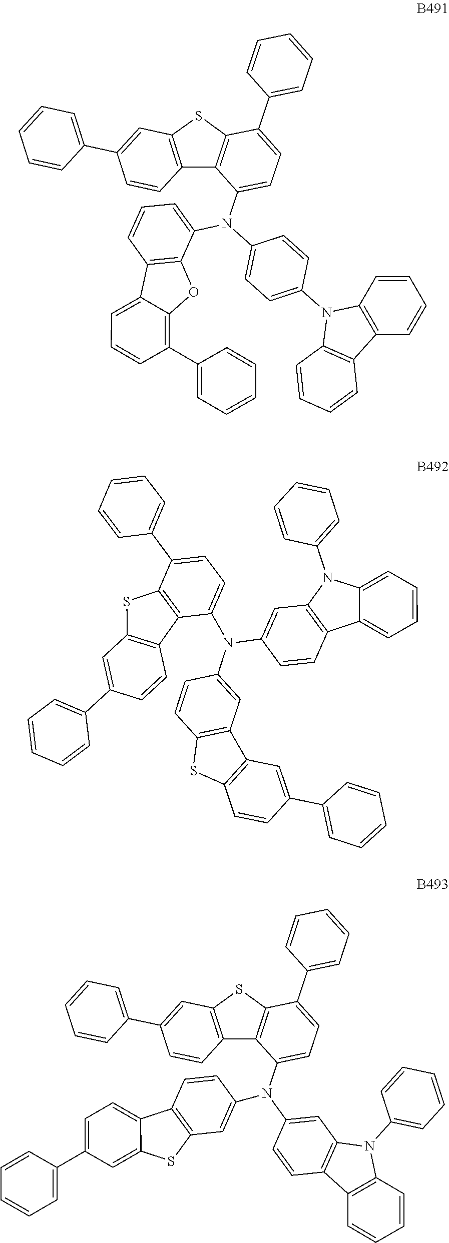 [SMILES: c1ccc(-c2ccc3c(c2)sc2c(-c4ccccc4)ccc(N(c4ccc(-n5c6ccccc6c6ccccc65)cc4)c4cccc5c4oc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4ccc5c6ccccc6n(-c6ccccc6)c5c4)c23)cc1 ; c1ccc(-c2ccc3c(c2)sc2cc(N(c4ccc5c6ccccc6n(-c6ccccc6)c5c4)c4ccc(-c5ccccc5)c5sc6cc(-c7ccccc7)ccc6c45)ccc23)cc1]

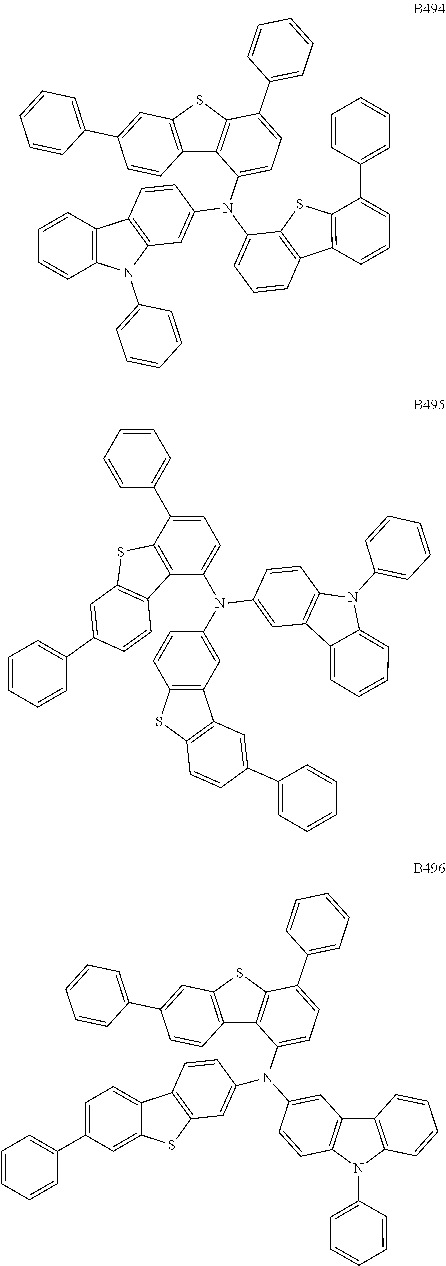 [SMILES: c1ccc(-c2ccc3c(c2)sc2c(-c4ccccc4)ccc(N(c4ccc5c6ccccc6n(-c6ccccc6)c5c4)c4cccc5c4sc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4ccc5c(c4)c4ccccc4n5-c4ccccc4)c23)cc1 ; c1ccc(-c2ccc3c(c2)sc2cc(N(c4ccc5c(c4)c4ccccc4n5-c4ccccc4)c4ccc(-c5ccccc5)c5sc6cc(-c7ccccc7)ccc6c45)ccc23)cc1]

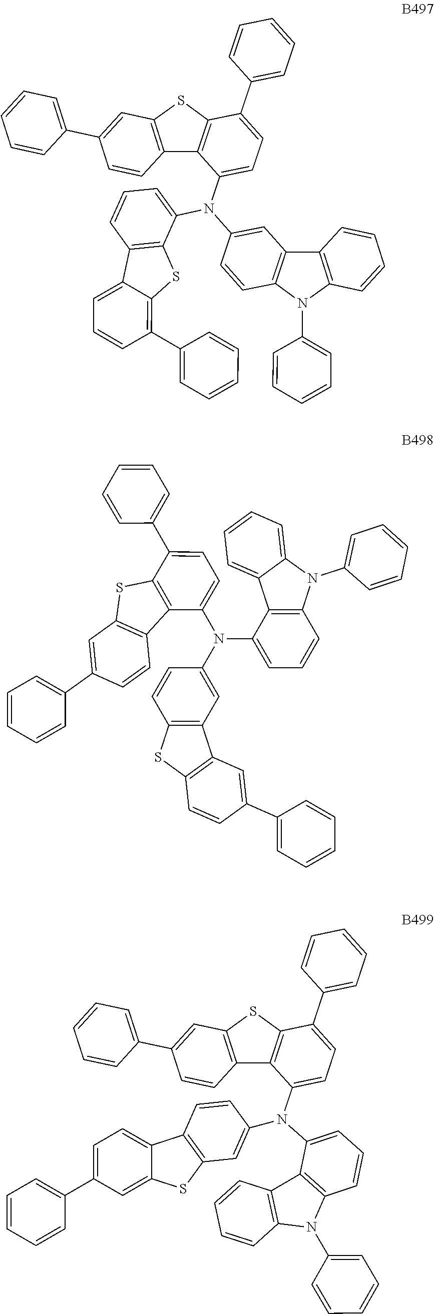 [SMILES: c1ccc(-c2ccc3c(c2)sc2c(-c4ccccc4)ccc(N(c4ccc5c(c4)c4ccccc4n5-c4ccccc4)c4cccc5c4sc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4cccc5c4c4ccccc4n5-c4ccccc4)c23)cc1 ; c1ccc(-c2ccc3c(c2)sc2cc(N(c4ccc(-c5ccccc5)c5sc6cc(-c7ccccc7)ccc6c45)c4cccc5c4c4ccccc4n5-c4ccccc4)ccc23)cc1]

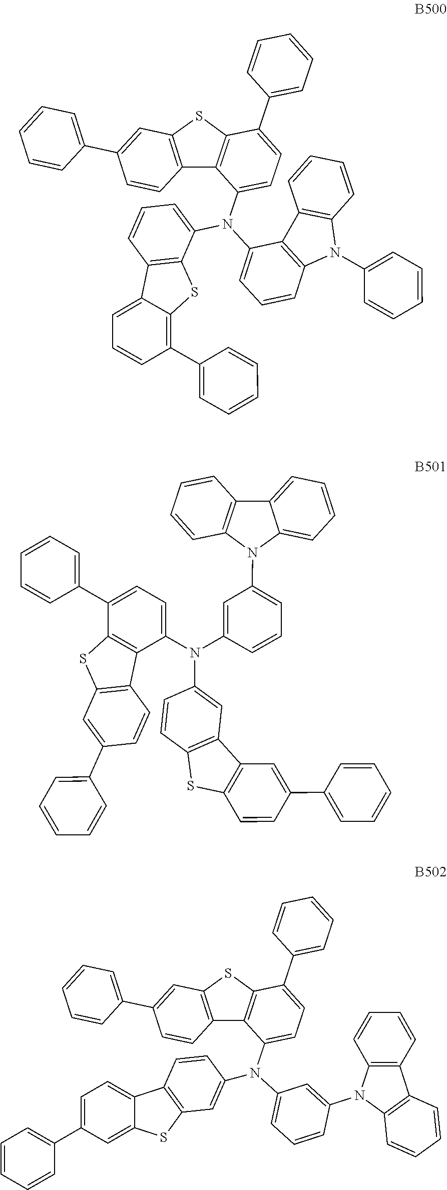 [SMILES: c1ccc(-c2ccc3c(c2)sc2c(-c4ccccc4)ccc(N(c4cccc5c4sc4c(-c6ccccc6)cccc45)c4cccc5c4c4ccccc4n5-c4ccccc4)c23)cc1 ; c1ccc(-c2ccc3c(c2)sc2c(-c4ccccc4)ccc(N(c4cccc(-n5c6ccccc6c6ccccc65)c4)c4ccc5sc6ccc(-c7ccccc7)cc6c5c4)c23)cc1 ; c1ccc(-c2ccc3c(c2)sc2cc(N(c4cccc(-n5c6ccccc6c6ccccc65)c4)c4ccc(-c5ccccc5)c5sc6cc(-c7ccccc7)ccc6c45)ccc23)cc1]

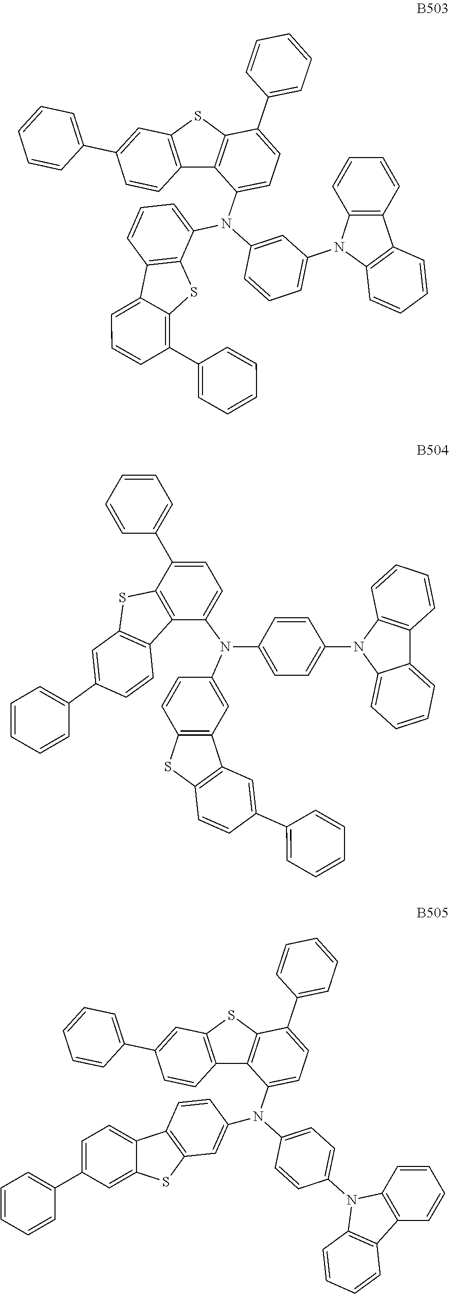 [SMILES: c1ccc(-c2ccc3c(c2)sc2c(-c4ccccc4)ccc(N(c4cccc(-n5c6ccccc6c6ccccc65)c4)c4cccc5c4sc4c(-c6ccccc6)cccc45)c23)cc1 ; c1ccc(-c2ccc3c(c2)sc2c(-c4ccccc4)ccc(N(c4ccc(-n5c6ccccc6c6ccccc65)cc4)c4ccc5sc6ccc(-c7ccccc7)cc6c5c4)c23)cc1 ; c1ccc(-c2ccc3c(c2)sc2cc(N(c4ccc(-n5c6ccccc6c6ccccc65)cc4)c4ccc(-c5ccccc5)c5sc6cc(-c7ccccc7)ccc6c45)ccc23)cc1]

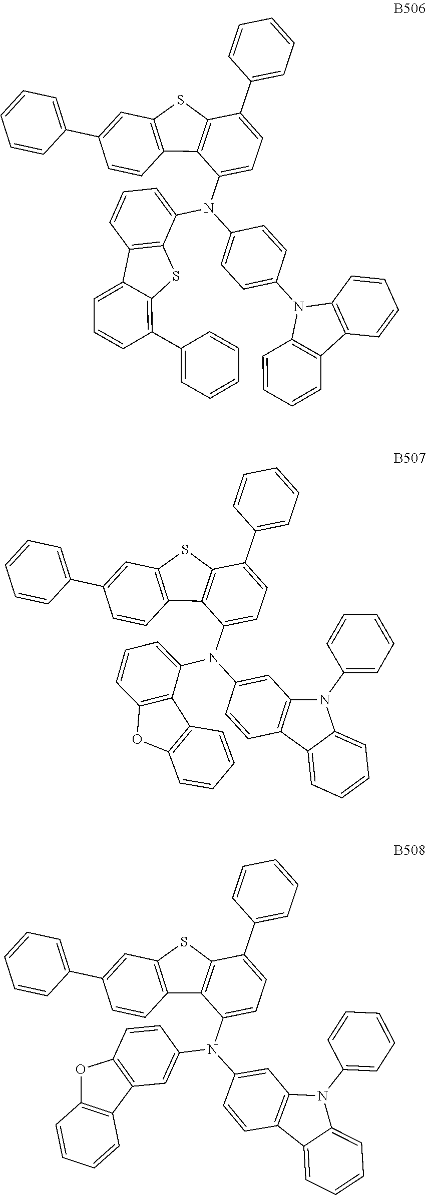 [SMILES: c1ccc(-c2ccc3c(c2)sc2c(-c4ccccc4)ccc(N(c4ccc(-n5c6ccccc6c6ccccc65)cc4)c4cccc5c4sc4c(-c6ccccc6)cccc45)c23)cc1 ; c1ccc(-c2ccc3c(c2)sc2c(-c4ccccc4)ccc(N(c4ccc5c6ccccc6n(-c6ccccc6)c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4ccc5c6ccccc6n(-c6ccccc6)c5c4)c23)cc1]

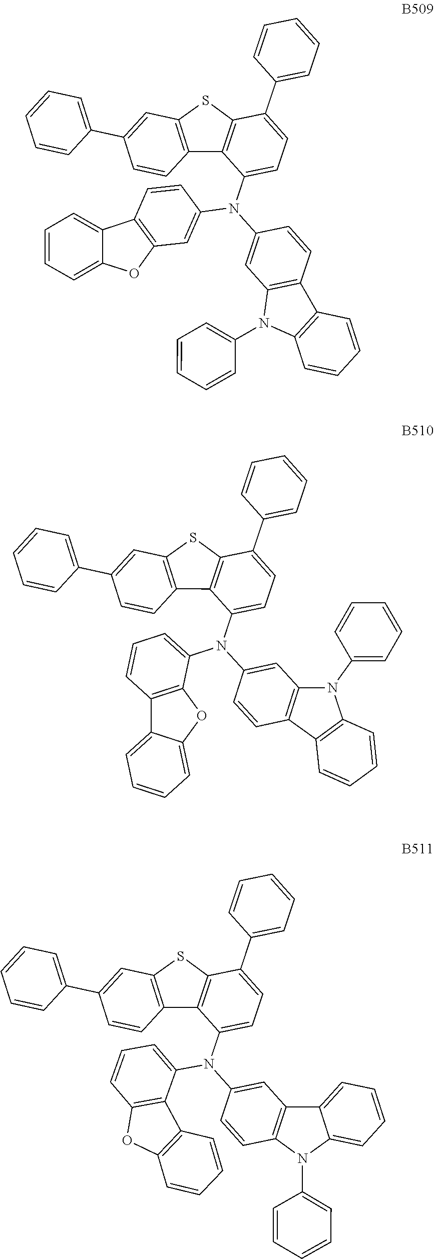 [SMILES: c1ccc(-c2ccc3c(c2)sc2c(-c4ccccc4)ccc(N(c4ccc5c(c4)oc4ccccc45)c4ccc5c6ccccc6n(-c6ccccc6)c5c4)c23)cc1 ; c1ccc(-c2ccc3c(c2)sc2c(-c4ccccc4)ccc(N(c4ccc5c6ccccc6n(-c6ccccc6)c5c4)c4cccc5c4oc4ccccc45)c23)cc1 ; c1ccc(-c2ccc3c(c2)sc2c(-c4ccccc4)ccc(N(c4ccc5c(c4)c4ccccc4n5-c4ccccc4)c4cccc5oc6ccccc6c45)c23)cc1]

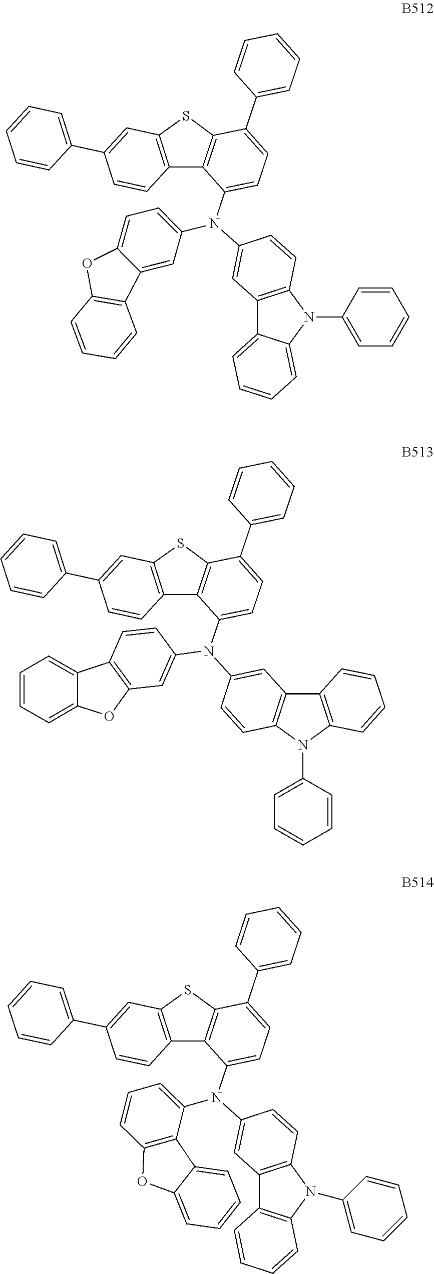 [SMILES: c1ccc(-c2ccc3c(c2)sc2c(-c4ccccc4)ccc(N(c4ccc5oc6ccccc6c5c4)c4ccc5c(c4)c4ccccc4n5-c4ccccc4)c23)cc1 ; c1ccc(-c2ccc3c(c2)sc2c(-c4ccccc4)ccc(N(c4ccc5c(c4)oc4ccccc45)c4ccc5c(c4)c4ccccc4n5-c4ccccc4)c23)cc1 ; c1ccc(-c2ccc3c(c2)sc2c(-c4ccccc4)ccc(N(c4ccc5c(c4)c4ccccc4n5-c4ccccc4)c4cccc5oc6ccccc6c45)c23)cc1]

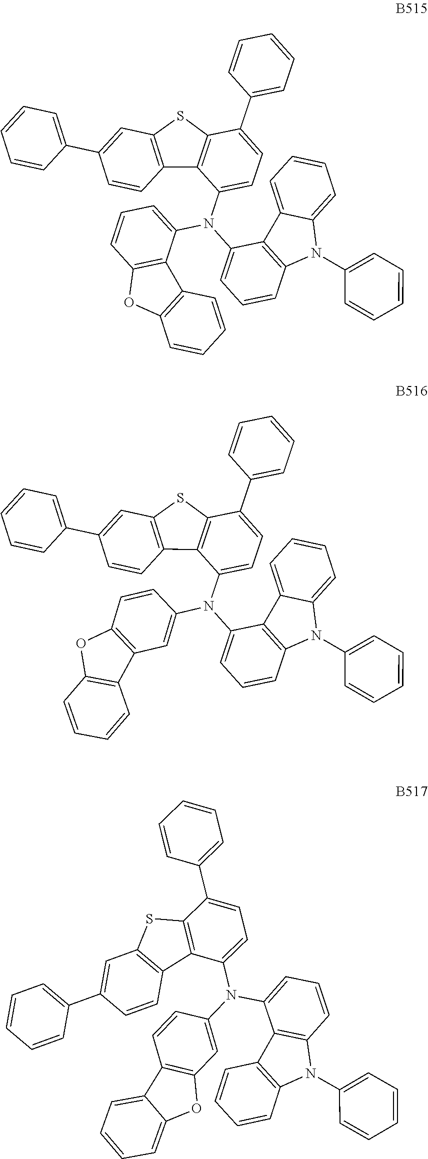 [SMILES: c1ccc(-c2ccc3c(c2)sc2c(-c4ccccc4)ccc(N(c4cccc5oc6ccccc6c45)c4cccc5c4c4ccccc4n5-c4ccccc4)c23)cc1 ; c1ccc(-c2ccc3c(c2)sc2c(-c4ccccc4)ccc(N(c4ccc5oc6ccccc6c5c4)c4cccc5c4c4ccccc4n5-c4ccccc4)c23)cc1 ; c1ccc(-c2ccc3c(c2)sc2c(-c4ccccc4)ccc(N(c4ccc5c(c4)oc4ccccc45)c4cccc5c4c4ccccc4n5-c4ccccc4)c23)cc1]

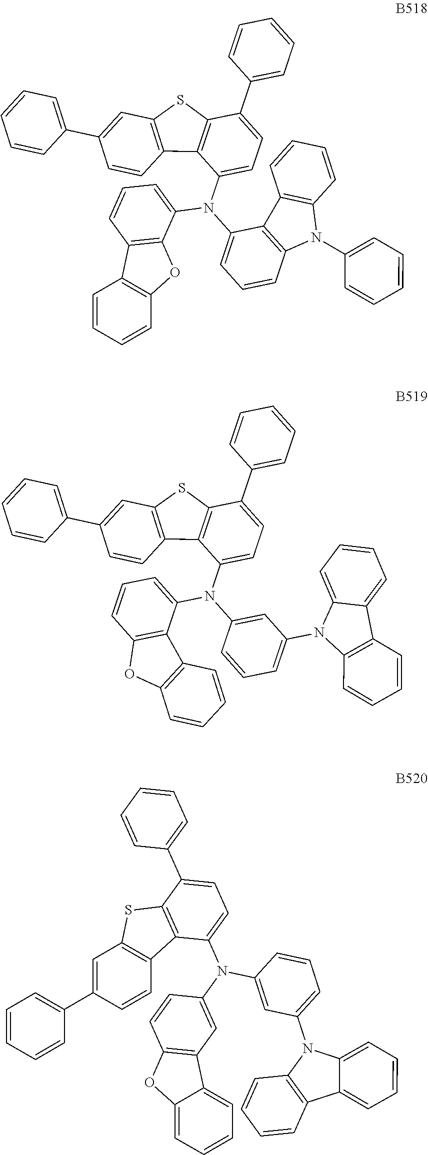 [SMILES: c1ccc(-c2ccc3c(c2)sc2c(-c4ccccc4)ccc(N(c4cccc5c4oc4ccccc45)c4cccc5c4c4ccccc4n5-c4ccccc4)c23)cc1 ; c1ccc(-c2ccc3c(c2)sc2c(-c4ccccc4)ccc(N(c4cccc(-n5c6ccccc6c6ccccc65)c4)c4cccc5oc6ccccc6c45)c23)cc1 ; c1ccc(-c2ccc3c(c2)sc2c(-c4ccccc4)ccc(N(c4cccc(-n5c6ccccc6c6ccccc65)c4)c4ccc5oc6ccccc6c5c4)c23)cc1]

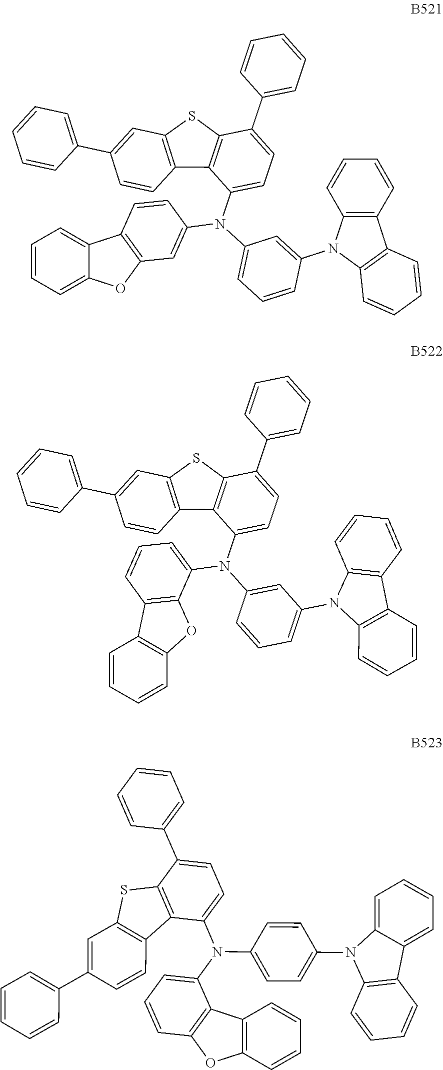 [SMILES: c1ccc(-c2ccc3c(c2)sc2c(-c4ccccc4)ccc(N(c4cccc(-n5c6ccccc6c6ccccc65)c4)c4ccc5c(c4)oc4ccccc45)c23)cc1 ; c1ccc(-c2ccc3c(c2)sc2c(-c4ccccc4)ccc(N(c4cccc(-n5c6ccccc6c6ccccc65)c4)c4cccc5c4oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5oc6ccccc6c45)c23)cc1]

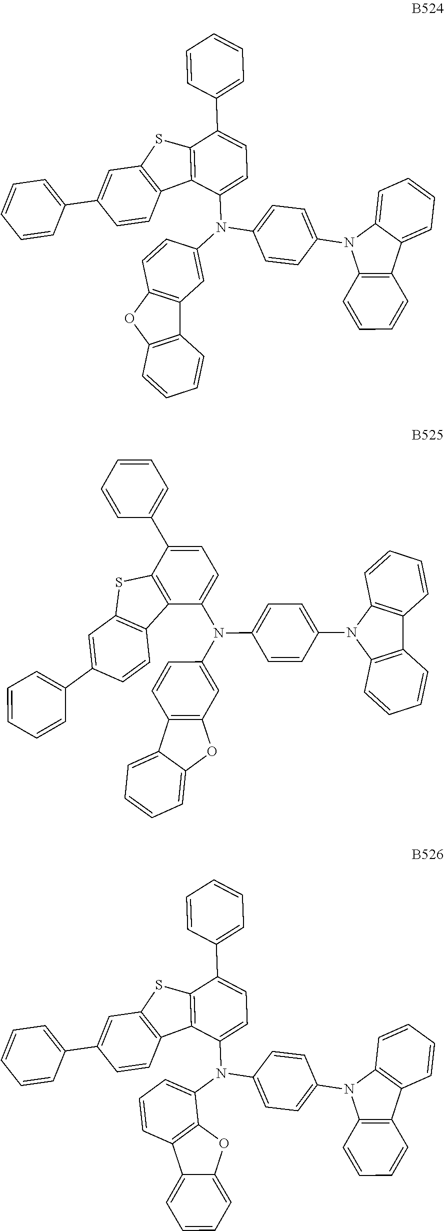 [SMILES: c1ccc(-c2ccc3c(c2)sc2c(-c4ccccc4)ccc(N(c4ccc(-n5c6ccccc6c6ccccc65)cc4)c4ccc5oc6ccccc6c5c4)c23)cc1 ; c1ccc(-c2ccc3c(c2)sc2c(-c4ccccc4)ccc(N(c4ccc(-n5c6ccccc6c6ccccc65)cc4)c4ccc5c(c4)oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5c4oc4ccccc45)c23)cc1]

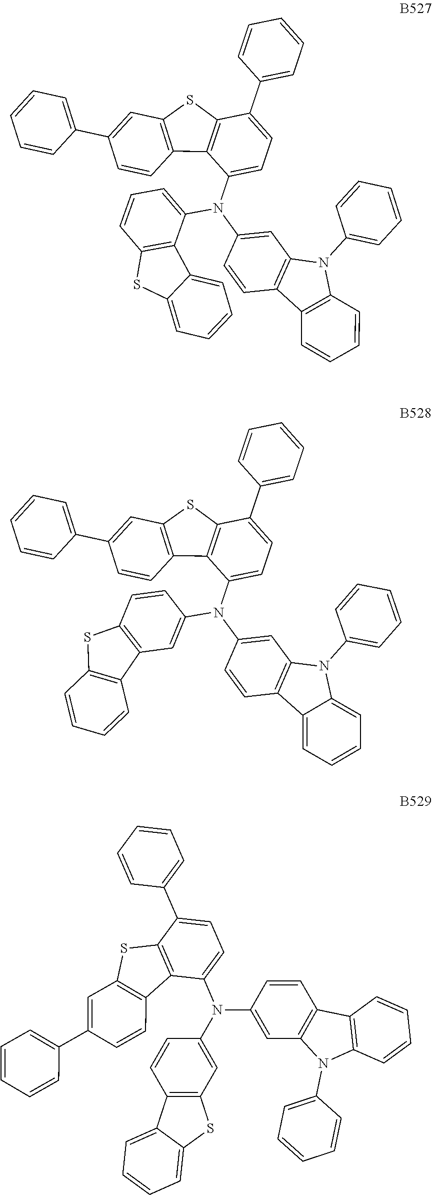 [SMILES: c1ccc(-c2ccc3c(c2)sc2c(-c4ccccc4)ccc(N(c4ccc5c6ccccc6n(-c6ccccc6)c5c4)c4cccc5sc6ccccc6c45)c23)cc1 ; c1ccc(-c2ccc3c(c2)sc2c(-c4ccccc4)ccc(N(c4ccc5sc6ccccc6c5c4)c4ccc5c6ccccc6n(-c6ccccc6)c5c4)c23)cc1 ; c1ccc(-c2ccc3c(c2)sc2c(-c4ccccc4)ccc(N(c4ccc5c(c4)sc4ccccc45)c4ccc5c6ccccc6n(-c6ccccc6)c5c4)c23)cc1]

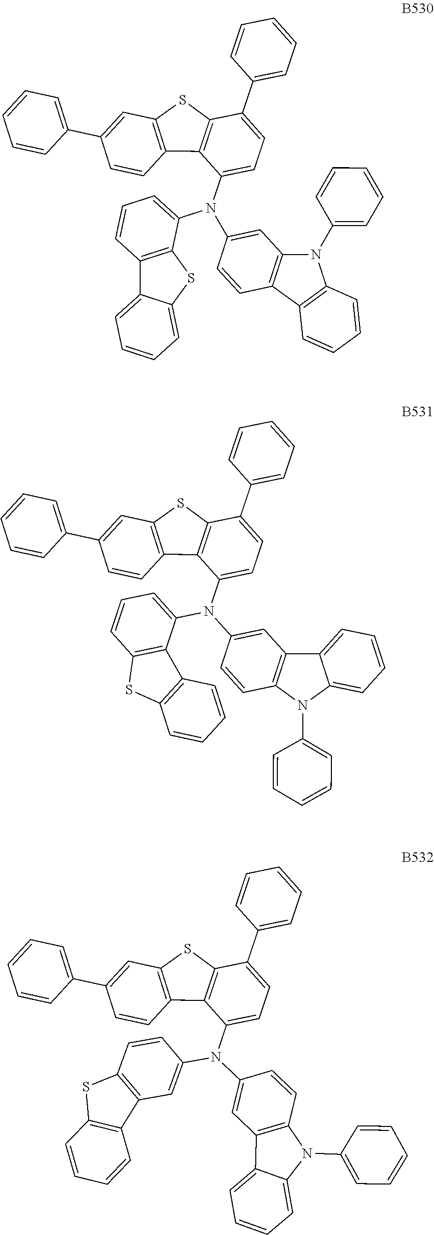 [SMILES: c1ccc(-c2ccc3c(c2)sc2c(-c4ccccc4)ccc(N(c4ccc5c6ccccc6n(-c6ccccc6)c5c4)c4cccc5c4sc4ccccc45)c23)cc1 ; c1ccc(-c2ccc3c(c2)sc2c(-c4ccccc4)ccc(N(c4ccc5c(c4)c4ccccc4n5-c4ccccc4)c4cccc5sc6ccccc6c45)c23)cc1 ; c1ccc(-c2ccc3c(c2)sc2c(-c4ccccc4)ccc(N(c4ccc5sc6ccccc6c5c4)c4ccc5c(c4)c4ccccc4n5-c4ccccc4)c23)cc1]

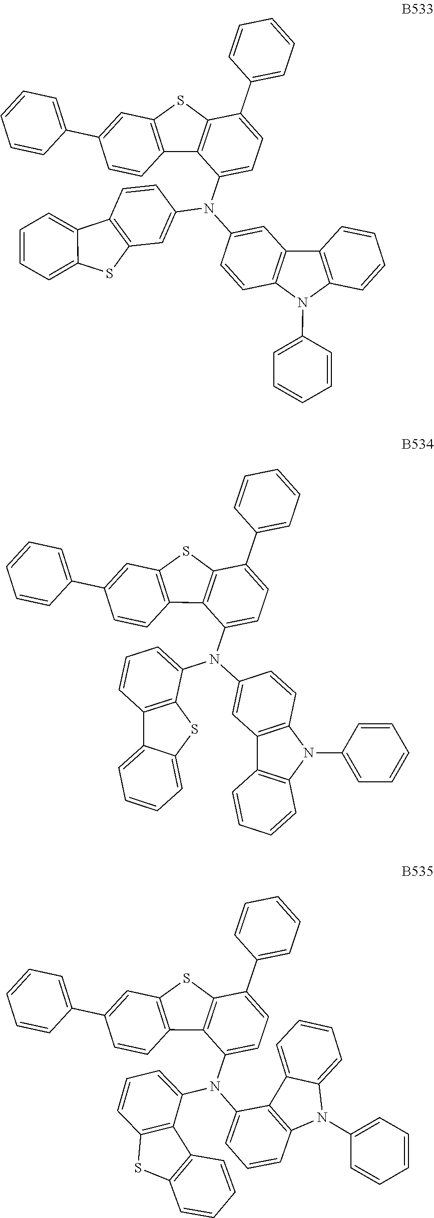 [SMILES: c1ccc(-c2ccc3c(c2)sc2c(-c4ccccc4)ccc(N(c4ccc5c(c4)sc4ccccc45)c4ccc5c(c4)c4ccccc4n5-c4ccccc4)c23)cc1 ; c1ccc(-c2ccc3c(c2)sc2c(-c4ccccc4)ccc(N(c4ccc5c(c4)c4ccccc4n5-c4ccccc4)c4cccc5c4sc4ccccc45)c23)cc1 ; c1ccc(-c2ccc3c(c2)sc2c(-c4ccccc4)ccc(N(c4cccc5sc6ccccc6c45)c4cccc5c4c4ccccc4n5-c4ccccc4)c23)cc1]

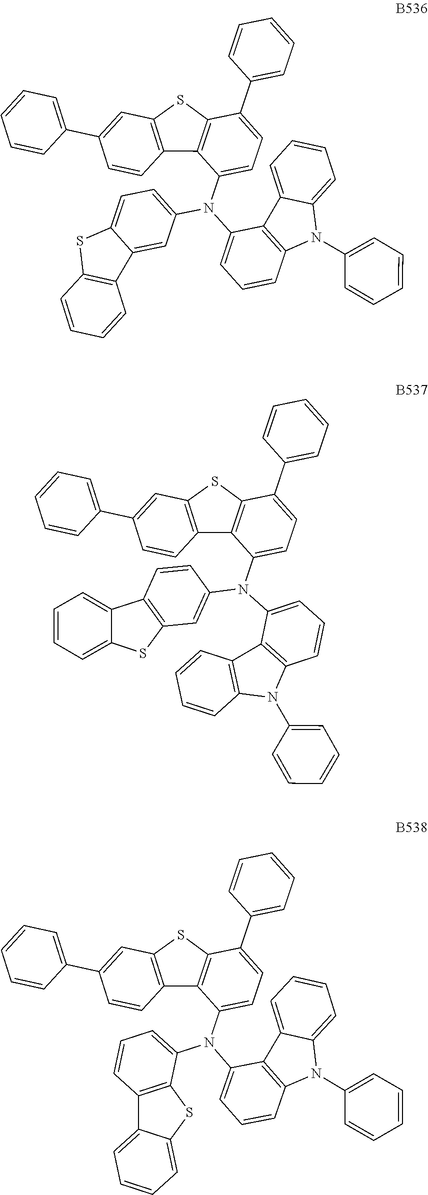 [SMILES: c1ccc(-c2ccc3c(c2)sc2c(-c4ccccc4)ccc(N(c4ccc5sc6ccccc6c5c4)c4cccc5c4c4ccccc4n5-c4ccccc4)c23)cc1 ; c1ccc(-c2ccc3c(c2)sc2c(-c4ccccc4)ccc(N(c4ccc5c(c4)sc4ccccc45)c4cccc5c4c4ccccc4n5-c4ccccc4)c23)cc1 ; c1ccc(-c2ccc3c(c2)sc2c(-c4ccccc4)ccc(N(c4cccc5c4sc4ccccc45)c4cccc5c4c4ccccc4n5-c4ccccc4)c23)cc1]

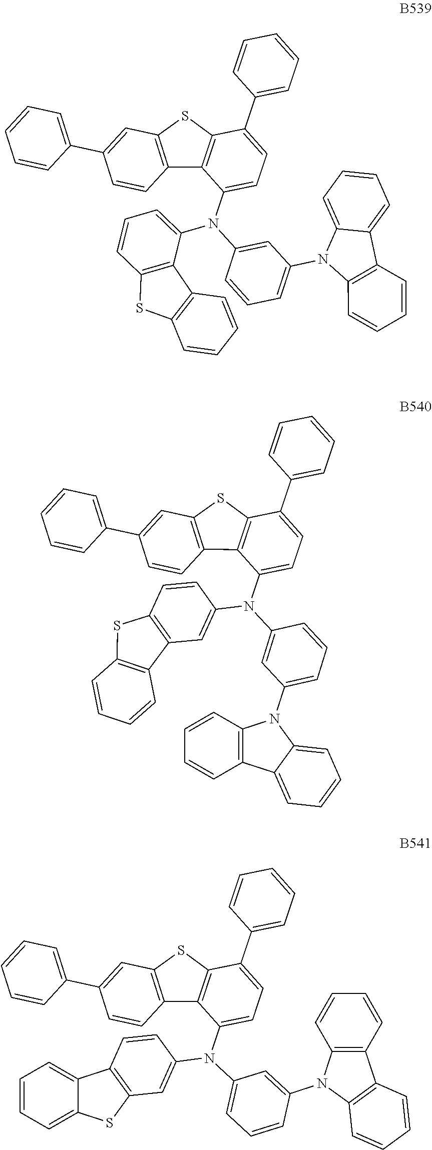 [SMILES: c1ccc(-c2ccc3c(c2)sc2c(-c4ccccc4)ccc(N(c4cccc(-n5c6ccccc6c6ccccc65)c4)c4cccc5sc6ccccc6c45)c23)cc1 ; c1ccc(-c2ccc3c(c2)sc2c(-c4ccccc4)ccc(N(c4cccc(-n5c6ccccc6c6ccccc65)c4)c4ccc5sc6ccccc6c5c4)c23)cc1 ; c1ccc(-c2ccc3c(c2)sc2c(-c4ccccc4)ccc(N(c4cccc(-n5c6ccccc6c6ccccc65)c4)c4ccc5c(c4)sc4ccccc45)c23)cc1]

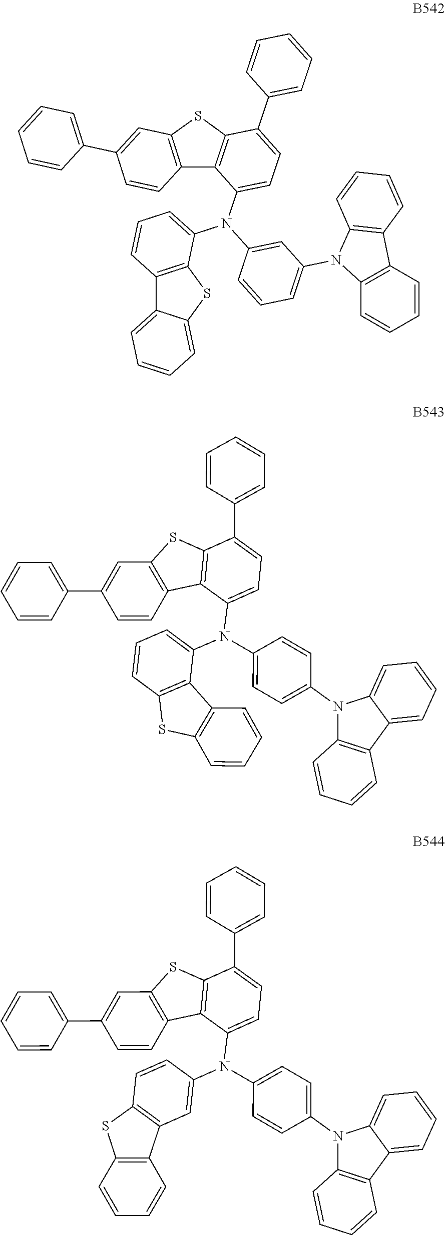 [SMILES: c1ccc(-c2ccc3c(c2)sc2c(-c4ccccc4)ccc(N(c4cccc(-n5c6ccccc6c6ccccc65)c4)c4cccc5c4sc4ccccc45)c23)cc1 ; c1ccc(-c2ccc3c(c2)sc2c(-c4ccccc4)ccc(N(c4ccc(-n5c6ccccc6c6ccccc65)cc4)c4cccc5sc6ccccc6c45)c23)cc1 ; c1ccc(-c2ccc3c(c2)sc2c(-c4ccccc4)ccc(N(c4ccc(-n5c6ccccc6c6ccccc65)cc4)c4ccc5sc6ccccc6c5c4)c23)cc1]

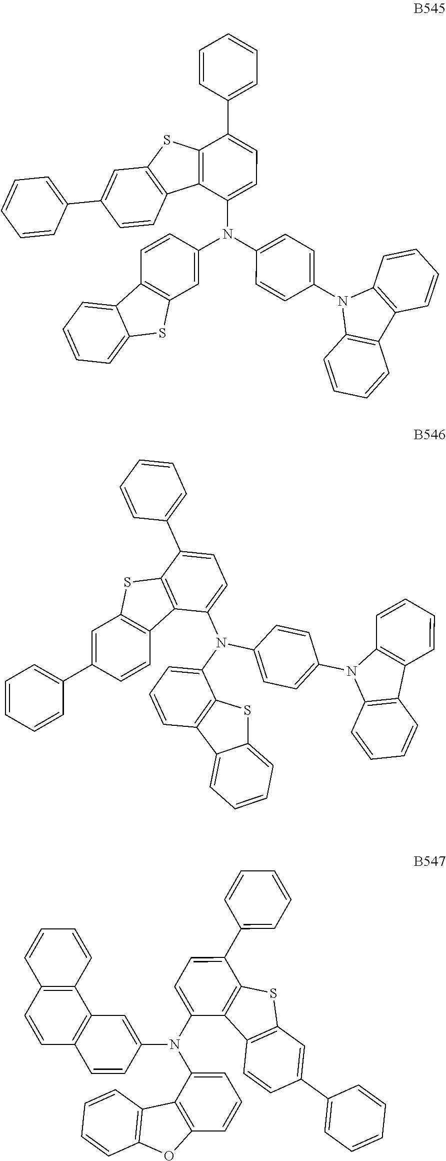 [SMILES: c1ccc(-c2ccc3c(c2)sc2c(-c4ccccc4)ccc(N(c4ccc(-n5c6ccccc6c6ccccc65)cc4)c4ccc5c(c4)sc4ccccc45)c23)cc1 ; c1ccc(-c2ccc3c(c2)sc2c(-c4ccccc4)ccc(N(c4ccc(-n5c6ccccc6c6ccccc65)cc4)c4cccc5c4sc4ccccc45)c23)cc1 ; c1ccc(-c2ccc3c(c2)sc2c(-c4ccccc4)ccc(N(c4ccc5ccc6ccccc6c5c4)c4cccc5oc6ccccc6c45)c23)cc1]

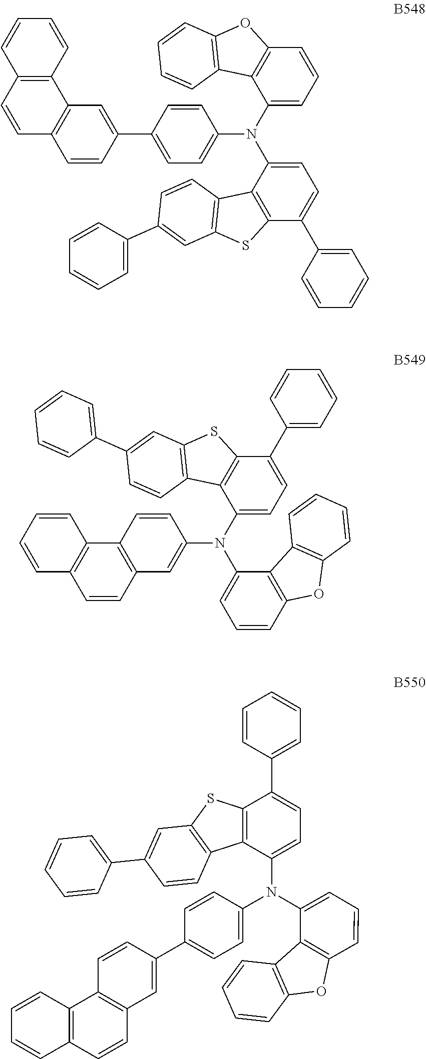 [SMILES: c1ccc(-c2ccc3c(c2)sc2c(-c4ccccc4)ccc(N(c4ccc(-c5ccc6ccc7ccccc7c6c5)cc4)c4cccc5oc6ccccc6c45)c23)cc1 ; c1ccc(-c2ccc3c(c2)sc2c(-c4ccccc4)ccc(N(c4ccc5c(ccc6ccccc65)c4)c4cccc5oc6ccccc6c45)c23)cc1 ; c1ccc(-c2ccc3c(c2)sc2c(-c4ccccc4)ccc(N(c4ccc(-c5ccc6c(ccc7ccccc76)c5)cc4)c4cccc5oc6ccccc6c45)c23)cc1]

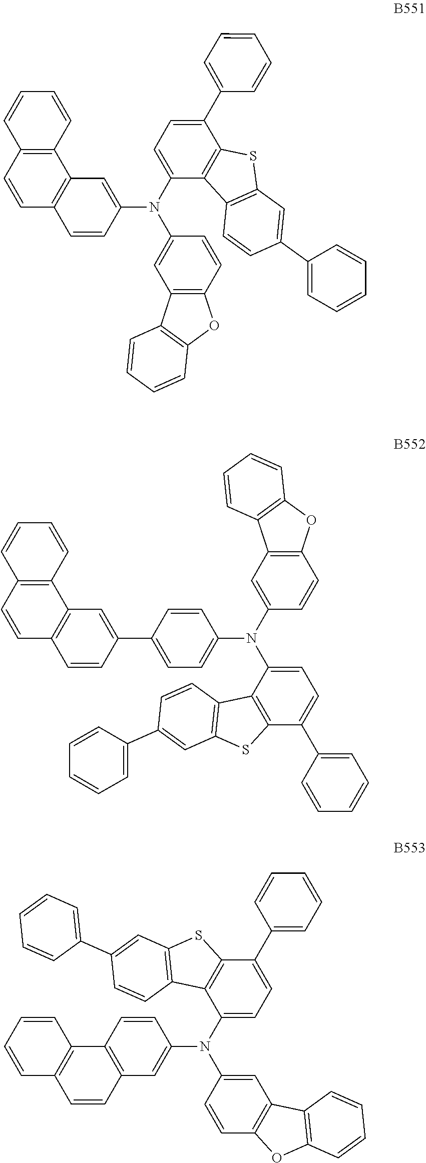 [SMILES: c1ccc(-c2ccc3c(c2)sc2c(-c4ccccc4)ccc(N(c4ccc5ccc6ccccc6c5c4)c4ccc5oc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5oc6ccccc6c5c4)c23)cc1 ; c1ccc(-c2ccc3c(c2)sc2c(-c4ccccc4)ccc(N(c4ccc5c(ccc6ccccc65)c4)c4ccc5oc6ccccc6c5c4)c23)cc1]

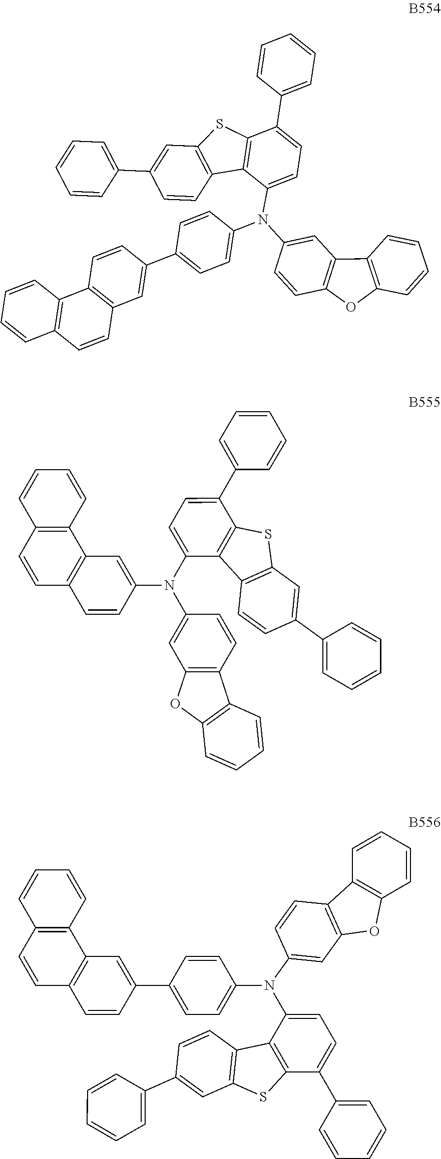 [SMILES: c1ccc(-c2ccc3c(c2)sc2c(-c4ccccc4)ccc(N(c4ccc(-c5ccc6c(ccc7ccccc76)c5)cc4)c4ccc5oc6ccccc6c5c4)c23)cc1 ; c1ccc(-c2ccc3c(c2)sc2c(-c4ccccc4)ccc(N(c4ccc5c(c4)oc4ccccc45)c4ccc5ccc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5c(c4)oc4ccccc45)c23)cc1]

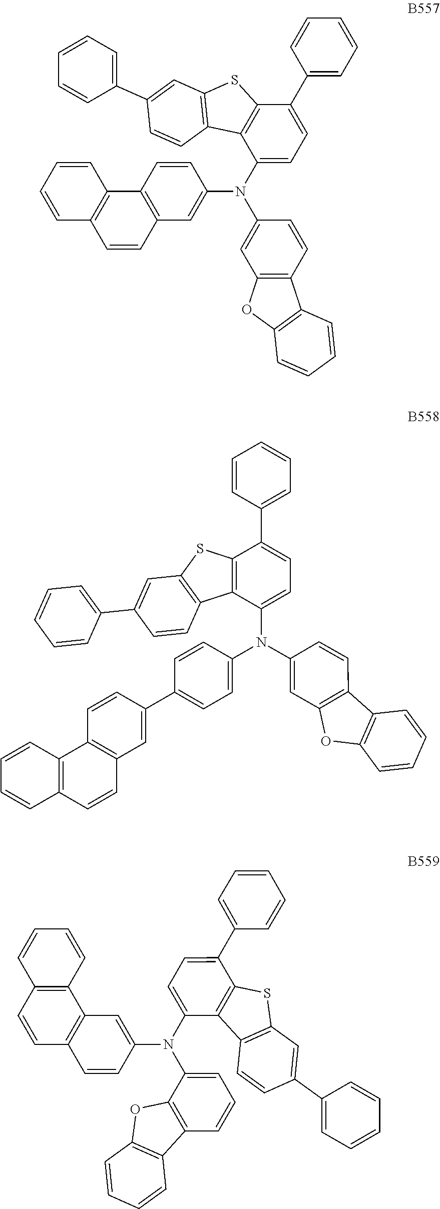 [SMILES: c1ccc(-c2ccc3c(c2)sc2c(-c4ccccc4)ccc(N(c4ccc5c(ccc6ccccc65)c4)c4ccc5c(c4)oc4ccccc45)c23)cc1 ; c1ccc(-c2ccc3c(c2)sc2c(-c4ccccc4)ccc(N(c4ccc(-c5ccc6c(ccc7ccccc76)c5)cc4)c4ccc5c(c4)oc4ccccc45)c23)cc1 ; c1ccc(-c2ccc3c(c2)sc2c(-c4ccccc4)ccc(N(c4ccc5ccc6ccccc6c5c4)c4cccc5c4oc4ccccc45)c23)cc1]

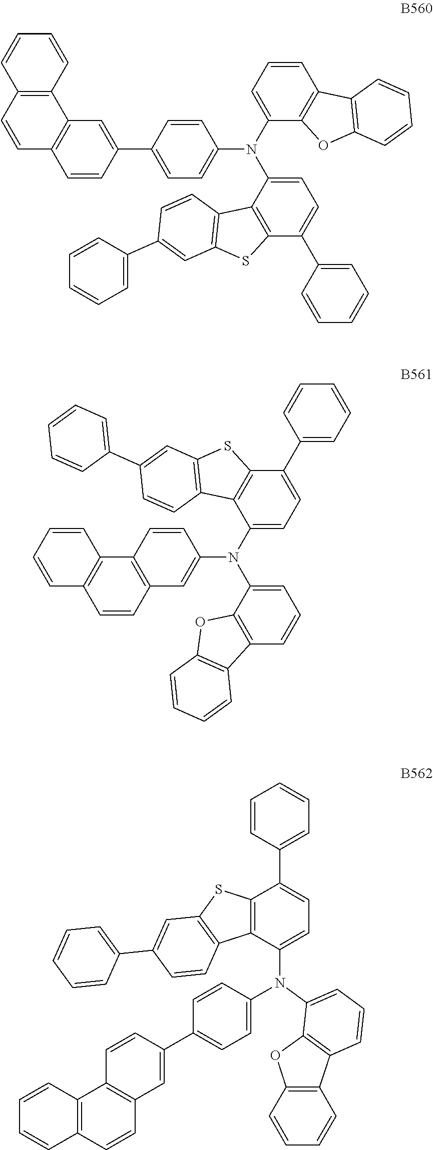 [SMILES: c1ccc(-c2ccc3c(c2)sc2c(-c4ccccc4)ccc(N(c4ccc(-c5ccc6ccc7ccccc7c6c5)cc4)c4cccc5c4oc4ccccc45)c23)cc1 ; c1ccc(-c2ccc3c(c2)sc2c(-c4ccccc4)ccc(N(c4ccc5c(ccc6ccccc65)c4)c4cccc5c4oc4ccccc45)c23)cc1 ; c1ccc(-c2ccc3c(c2)sc2c(-c4ccccc4)ccc(N(c4ccc(-c5ccc6c(ccc7ccccc76)c5)cc4)c4cccc5c4oc4ccccc45)c23)cc1]

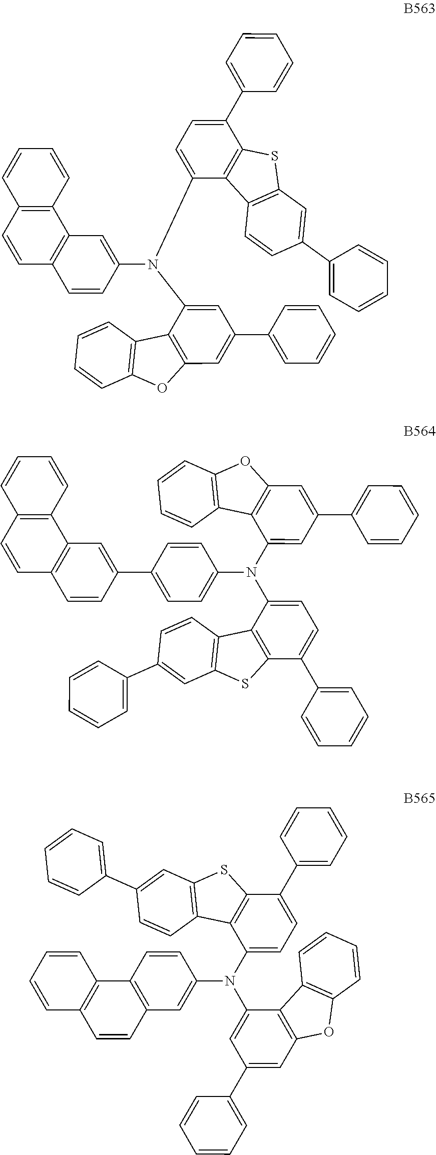 [SMILES: c1ccc(-c2cc(N(c3ccc4ccc5ccccc5c4c3)c3ccc(-c4ccccc4)c4sc5cc(-c6ccccc6)ccc5c34)c3c(c2)oc2ccccc23)cc1 ; c1ccc(-c2cc(N(c3ccc(-c4ccc5ccc6ccccc6c5c4)cc3)c3ccc(-c4ccccc4)c4sc5cc(-c6ccccc6)ccc5c34)c3c(c2)oc2ccccc23)cc1 ; c1ccc(-c2cc(N(c3ccc4c(ccc5ccccc54)c3)c3ccc(-c4ccccc4)c4sc5cc(-c6ccccc6)ccc5c34)c3c(c2)oc2ccccc23)cc1]

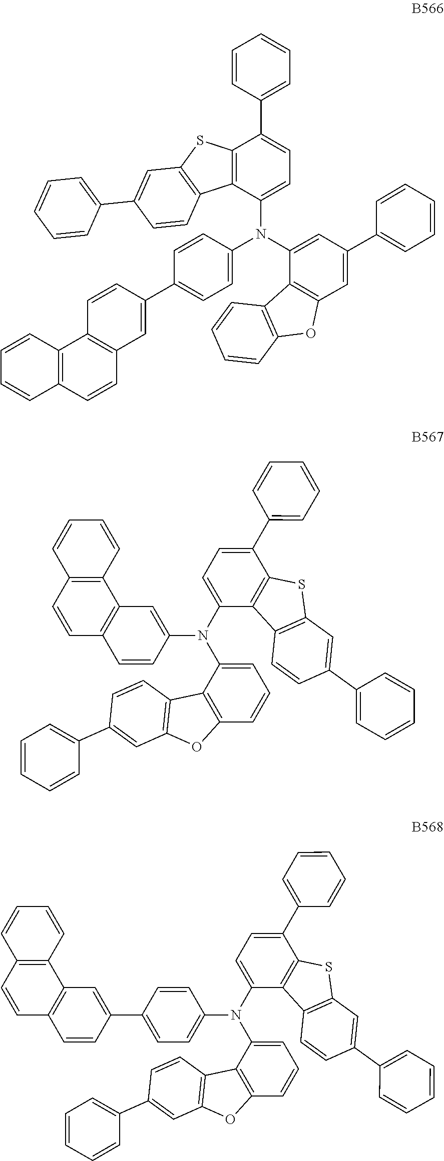 [SMILES: c1ccc(-c2cc(N(c3ccc(-c4ccc5c(ccc6ccccc65)c4)cc3)c3ccc(-c4ccccc4)c4sc5cc(-c6ccccc6)ccc5c34)c3c(c2)oc2ccccc23)cc1 ; c1ccc(-c2ccc3c(c2)oc2cccc(N(c4ccc5ccc6ccccc6c5c4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5sc6cc(-c7ccccc7)ccc6c45)c23)cc1]

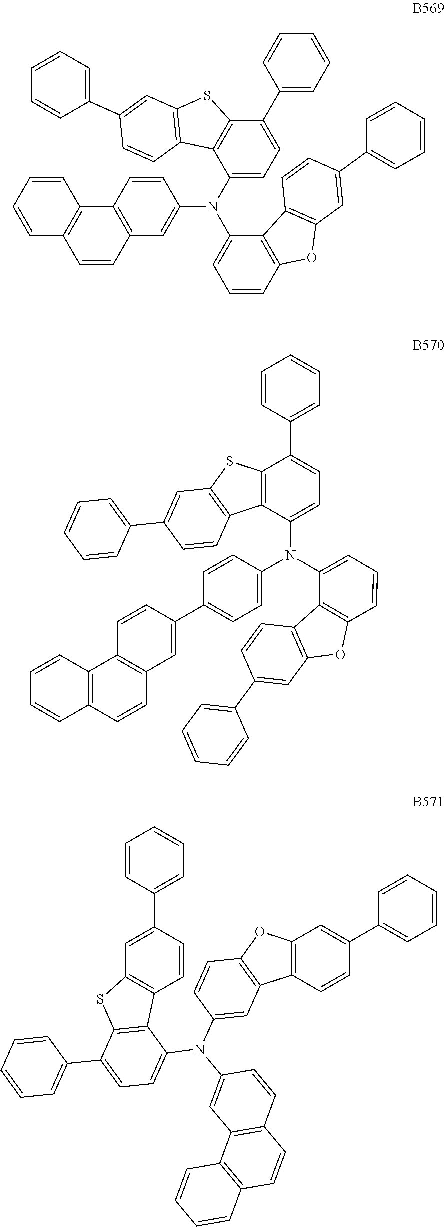 [SMILES: c1ccc(-c2ccc3c(c2)oc2cccc(N(c4ccc5c(ccc6ccccc65)c4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5sc6cc(-c7ccccc7)ccc6c45)c23)cc1 ; c1ccc(-c2ccc3c(c2)oc2ccc(N(c4ccc5ccc6ccccc6c5c4)c4ccc(-c5ccccc5)c5sc6cc(-c7ccccc7)ccc6c45)cc23)cc1]

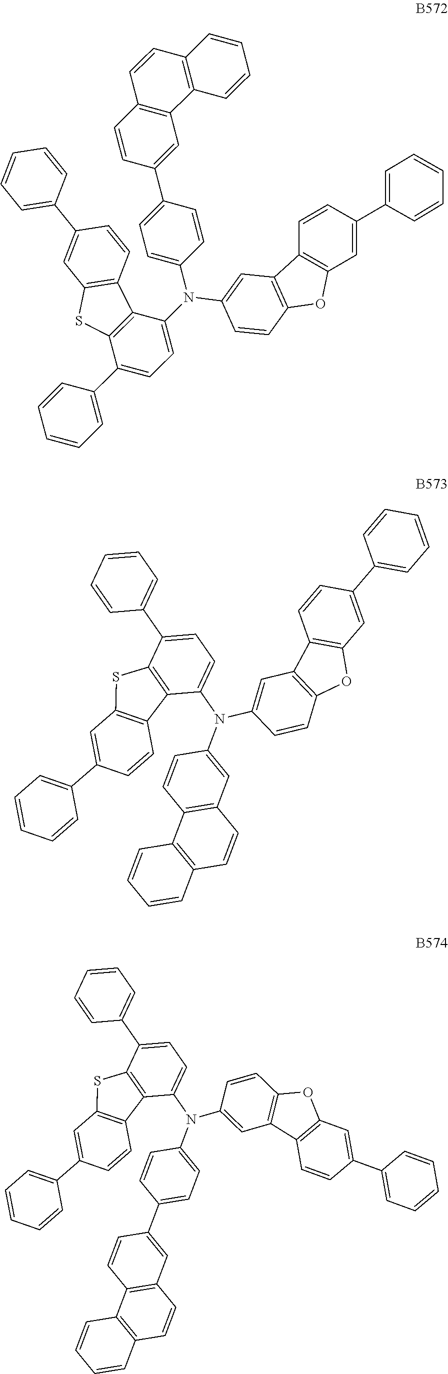 [SMILES: c1ccc(-c2ccc3c(c2)oc2ccc(N(c4ccc(-c5ccc6ccc7ccccc7c6c5)cc4)c4ccc(-c5ccccc5)c5sc6cc(-c7ccccc7)ccc6c45)cc23)cc1 ; c1ccc(-c2ccc3c(c2)oc2ccc(N(c4ccc5c(ccc6ccccc65)c4)c4ccc(-c5ccccc5)c5sc6cc(-c7ccccc7)ccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5sc6cc(-c7ccccc7)ccc6c45)cc23)cc1]

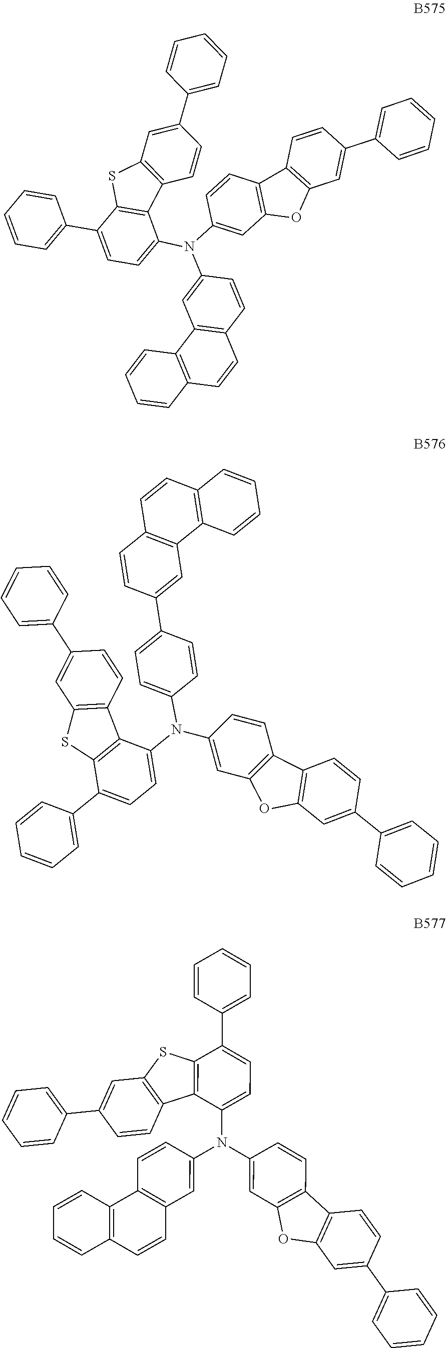 [SMILES: c1ccc(-c2ccc3c(c2)oc2cc(N(c4ccc5ccc6ccccc6c5c4)c4ccc(-c5ccccc5)c5sc6cc(-c7ccccc7)ccc6c45)ccc23)cc1 ; c1ccc(-c2ccc3c(c2)oc2cc(N(c4ccc(-c5ccc6ccc7ccccc7c6c5)cc4)c4ccc(-c5ccccc5)c5sc6cc(-c7ccccc7)ccc6c45)ccc23)cc1 ; c1ccc(-c2ccc3c(c2)oc2cc(N(c4ccc5c(ccc6ccccc65)c4)c4ccc(-c5ccccc5)c5sc6cc(-c7ccccc7)ccc6c45)ccc23)cc1]

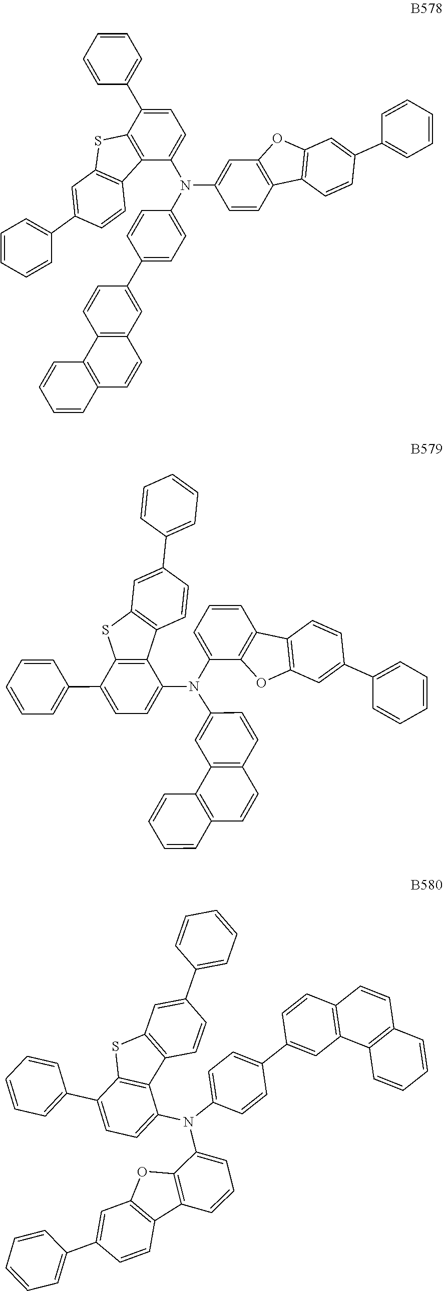 [SMILES: c1ccc(-c2ccc3c(c2)oc2cc(N(c4ccc(-c5ccc6c(ccc7ccccc76)c5)cc4)c4ccc(-c5ccccc5)c5sc6cc(-c7ccccc7)ccc6c45)ccc23)cc1 ; c1ccc(-c2ccc3c(c2)oc2c(N(c4ccc5ccc6ccccc6c5c4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5sc6cc(-c7ccccc7)ccc6c45)cccc23)cc1]

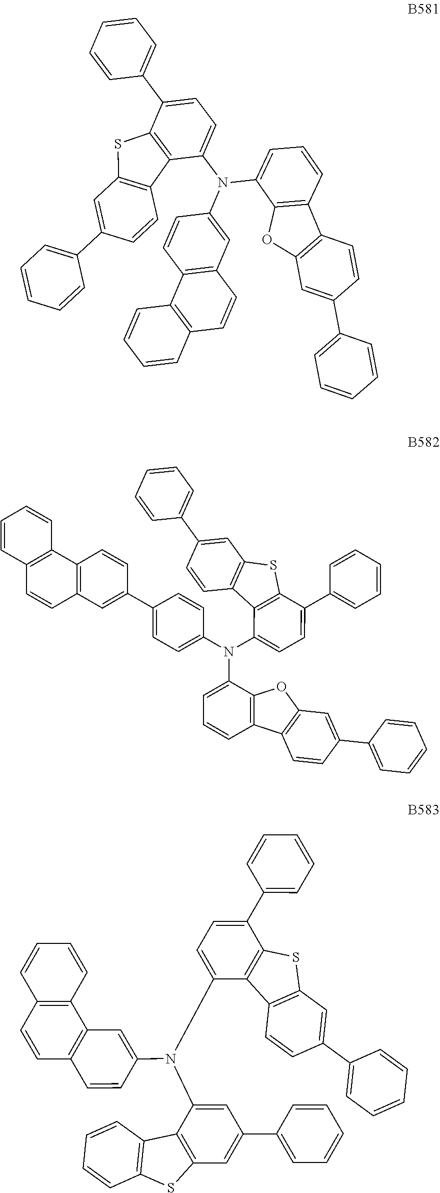 [SMILES: c1ccc(-c2ccc3c(c2)oc2c(N(c4ccc5c(ccc6ccccc65)c4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6c(ccc7ccccc76)c5)cc4)c4ccc(-c5ccccc5)c5sc6cc(-c7ccccc7)ccc6c45)cccc23)cc1 ; c1ccc(-c2ccc3c(c2)sc2c(-c4ccccc4)ccc(N(c4ccc5ccc6ccccc6c5c4)c4cc(-c5ccccc5)cc5sc6ccccc6c45)c23)cc1]

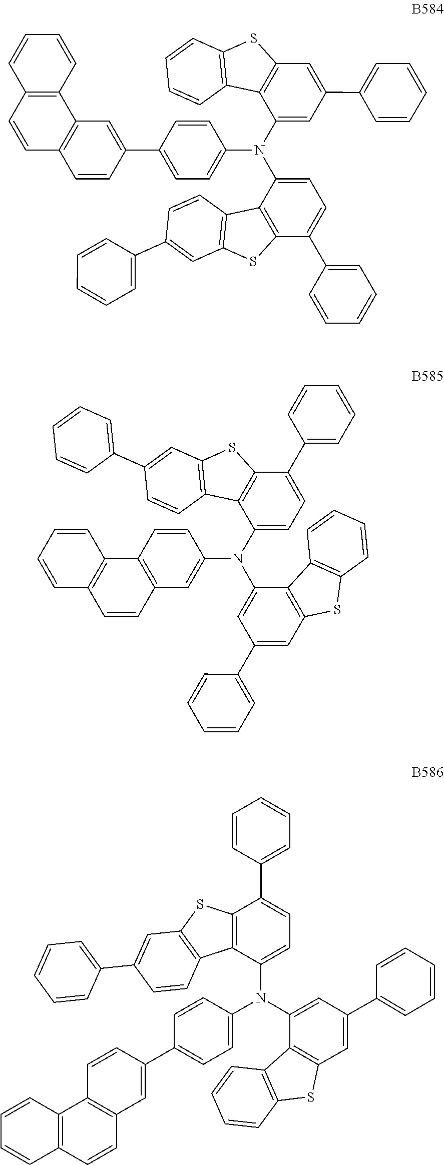 [SMILES: c1ccc(-c2ccc3c(c2)sc2c(-c4ccccc4)ccc(N(c4ccc(-c5ccc6ccc7ccccc7c6c5)cc4)c4cc(-c5ccccc5)cc5sc6ccccc6c45)c23)cc1 ; c1ccc(-c2ccc3c(c2)sc2c(-c4ccccc4)ccc(N(c4ccc5c(ccc6ccccc65)c4)c4cc(-c5ccccc5)cc5sc6ccccc6c45)c23)cc1 ; c1ccc(-c2ccc3c(c2)sc2c(-c4ccccc4)ccc(N(c4ccc(-c5ccc6c(ccc7ccccc76)c5)cc4)c4cc(-c5ccccc5)cc5sc6ccccc6c45)c23)cc1]

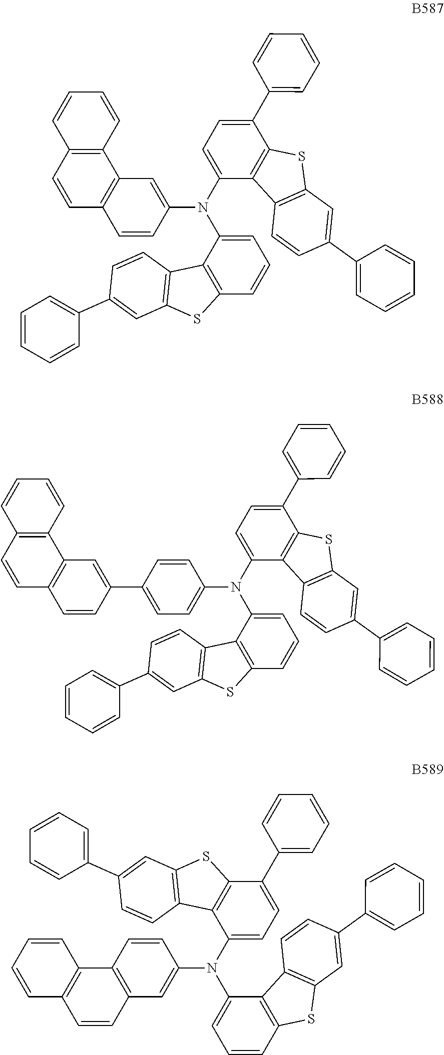 [SMILES: c1ccc(-c2ccc3c(c2)sc2cccc(N(c4ccc5ccc6ccccc6c5c4)c4ccc(-c5ccccc5)c5sc6cc(-c7ccccc7)ccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5sc6cc(-c7ccccc7)ccc6c45)c23)cc1 ; c1ccc(-c2ccc3c(c2)sc2cccc(N(c4ccc5c(ccc6ccccc65)c4)c4ccc(-c5ccccc5)c5sc6cc(-c7ccccc7)ccc6c45)c23)cc1]

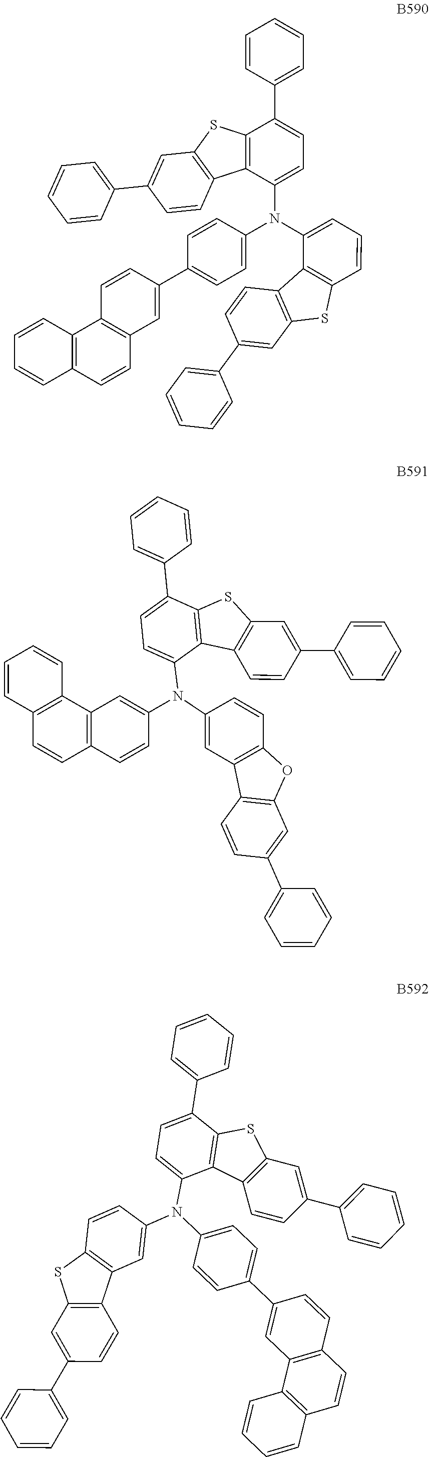 [SMILES: c1ccc(-c2ccc3c(c2)sc2cccc(N(c4ccc(-c5ccc6c(ccc7ccccc76)c5)cc4)c4ccc(-c5ccccc5)c5sc6cc(-c7ccccc7)ccc6c45)c23)cc1 ; c1ccc(-c2ccc3c(c2)oc2ccc(N(c4ccc5ccc6ccccc6c5c4)c4ccc(-c5ccccc5)c5sc6cc(-c7ccccc7)ccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5sc6cc(-c7ccccc7)ccc6c45)cc23)cc1]

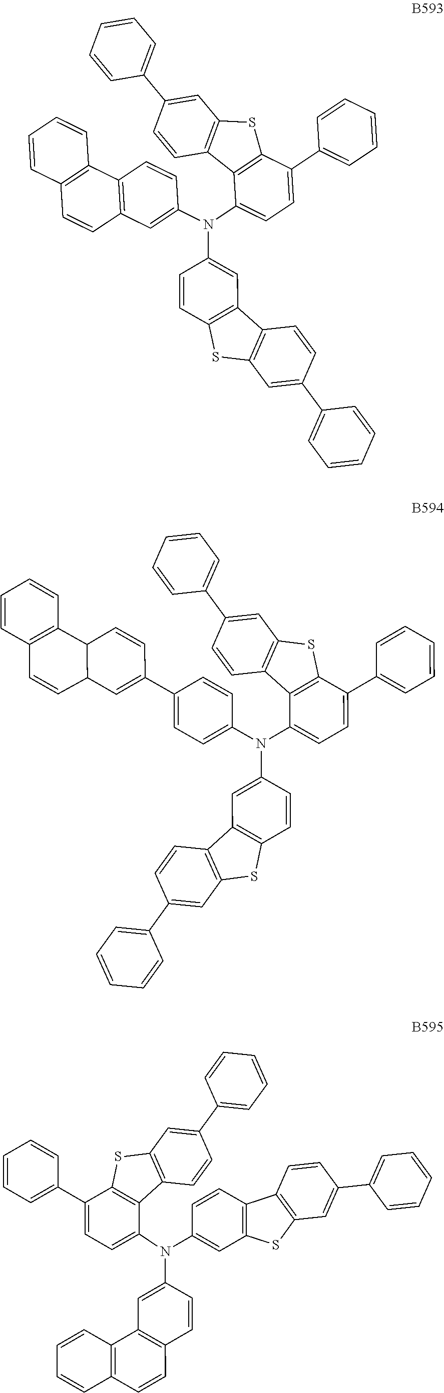 [SMILES: c1ccc(-c2ccc3c(c2)sc2ccc(N(c4ccc5c(ccc6ccccc65)c4)c4ccc(-c5ccccc5)c5sc6cc(-c7ccccc7)ccc6c45)cc23)cc1 ; C1=CC2c3ccccc3C=CC2C=C1c1ccc(N(c2ccc3sc4cc(-c5ccccc5)ccc4c3c2)c2ccc(-c3ccccc3)c3sc4cc(-c5ccccc5)ccc4c23)cc1 ; c1ccc(-c2ccc3c(c2)sc2cc(N(c4ccc5ccc6ccccc6c5c4)c4ccc(-c5ccccc5)c5sc6cc(-c7ccccc7)ccc6c45)ccc23)cc1]

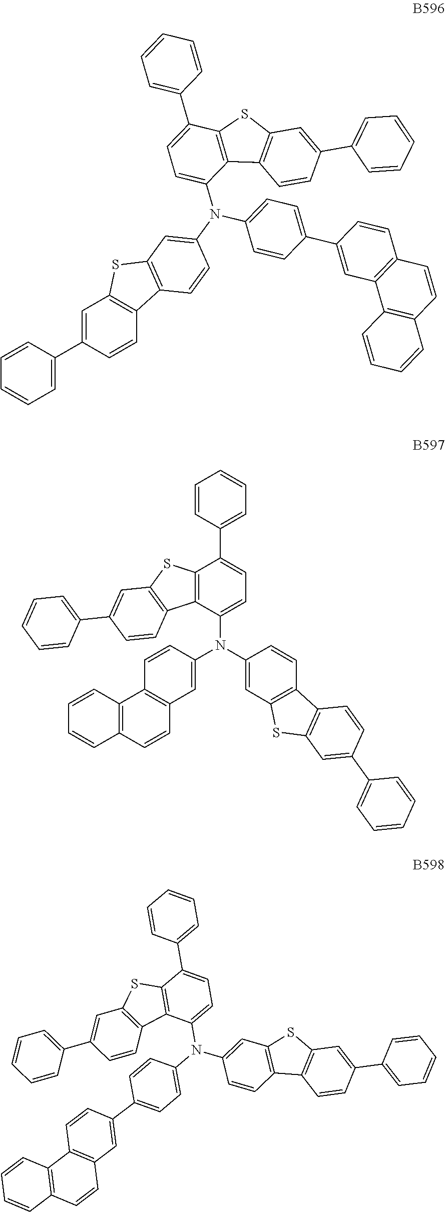 [SMILES: c1ccc(-c2ccc3c(c2)sc2cc(N(c4ccc(-c5ccc6ccc7ccccc7c6c5)cc4)c4ccc(-c5ccccc5)c5sc6cc(-c7ccccc7)ccc6c45)ccc23)cc1 ; c1ccc(-c2ccc3c(c2)sc2cc(N(c4ccc5c(ccc6ccccc65)c4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5sc6cc(-c7ccccc7)ccc6c45)ccc23)cc1]

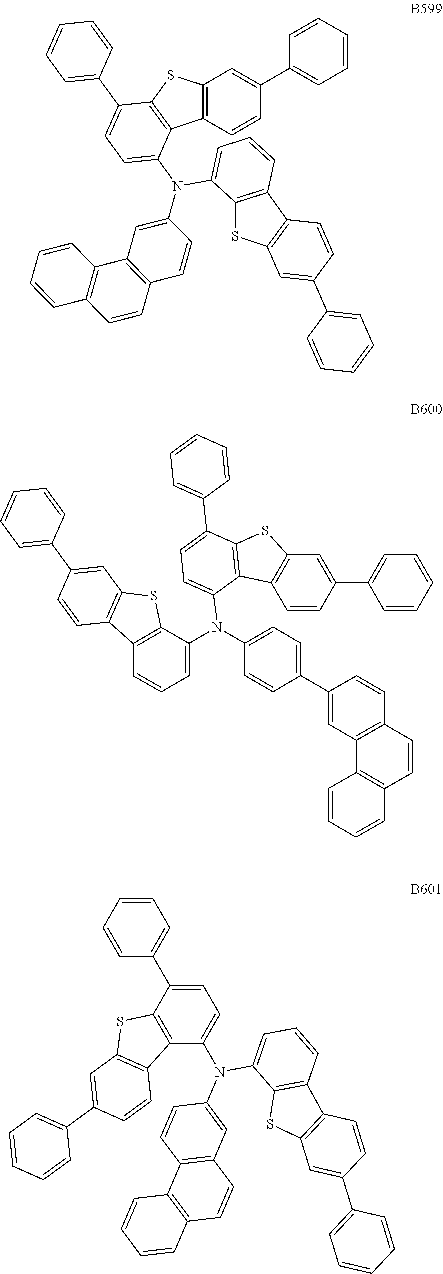 [SMILES: c1ccc(-c2ccc3c(c2)sc2c(N(c4ccc5ccc6ccccc6c5c4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5sc6cc(-c7ccccc7)ccc6c45)cccc23)cc1 ; c1ccc(-c2ccc3c(c2)sc2c(N(c4ccc5c(ccc6ccccc65)c4)c4ccc(-c5ccccc5)c5sc6cc(-c7ccccc7)ccc6c45)cccc23)cc1]

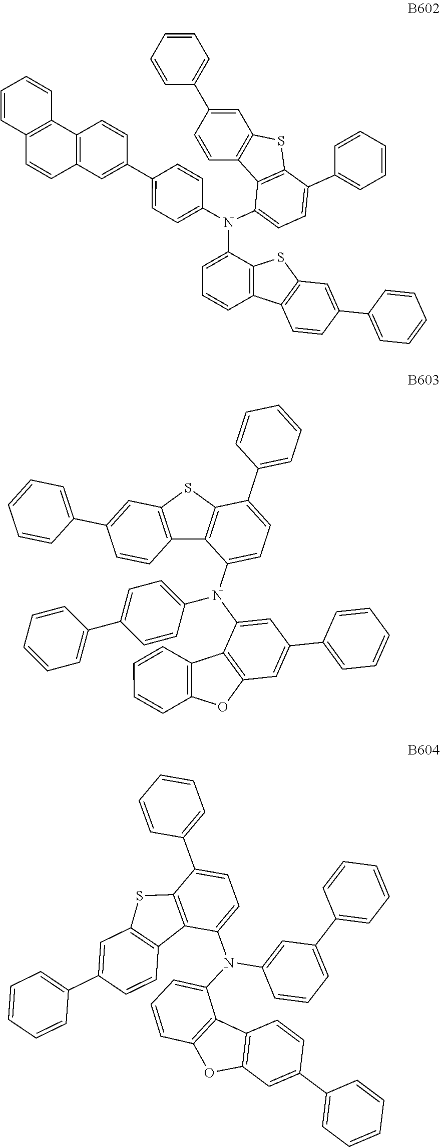 [SMILES: c1ccc(-c2ccc3c(c2)sc2c(N(c4ccc(-c5ccc6c(ccc7ccccc76)c5)cc4)c4ccc(-c5ccccc5)c5sc6cc(-c7ccccc7)ccc6c45)cccc23)cc1 ; c1ccc(-c2ccc(N(c3cc(-c4ccccc4)cc4oc5ccccc5c34)c3ccc(-c4ccccc4)c4sc5cc(-c6ccccc6)ccc5c34)cc2)cc1 ; c1ccc(-c2cccc(N(c3cccc4oc5cc(-c6ccccc6)ccc5c34)c3ccc(-c4ccccc4)c4sc5cc(-c6ccccc6)ccc5c34)c2)cc1]

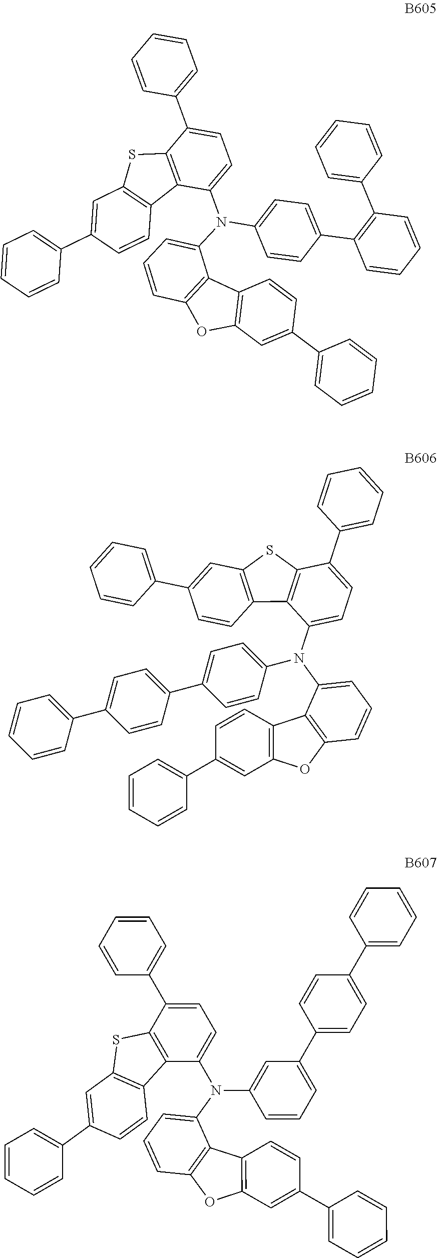 [SMILES: c1ccc(-c2ccc3c(c2)oc2cccc(N(c4ccc(-c5ccccc5-c5ccccc5)cc4)c4ccc(-c5ccccc5)c5sc6cc(-c7ccccc7)ccc6c45)c23)cc1 ; c1ccc(-c2ccc(-c3ccc(N(c4cccc5oc6cc(-c7ccccc7)ccc6c45)c4ccc(-c5ccccc5)c5sc6cc(-c7ccccc7)ccc6c45)cc3)cc2)cc1 ; c1ccc(-c2ccc(-c3cccc(N(c4cccc5oc6cc(-c7ccccc7)ccc6c45)c4ccc(-c5ccccc5)c5sc6cc(-c7ccccc7)ccc6c45)c3)cc2)cc1]

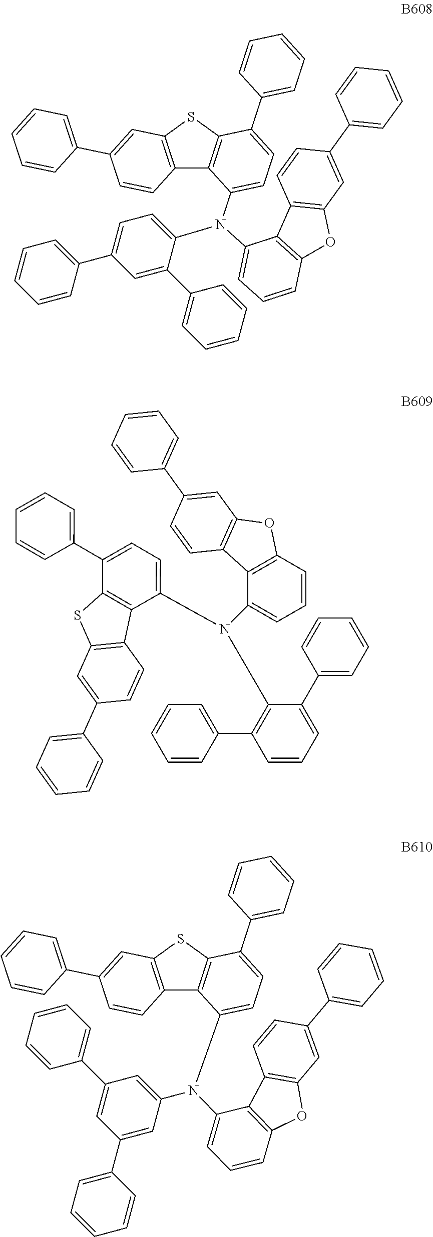 [SMILES: c1ccc(-c2ccc(N(c3cccc4oc5cc(-c6ccccc6)ccc5c34)c3ccc(-c4ccccc4)c4sc5cc(-c6ccccc6)ccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5sc6cc(-c7ccccc7)ccc6c45)c23)cc1 ; c1ccc(-c2cc(-c3ccccc3)cc(N(c3cccc4oc5cc(-c6ccccc6)ccc5c34)c3ccc(-c4ccccc4)c4sc5cc(-c6ccccc6)ccc5c34)c2)cc1]

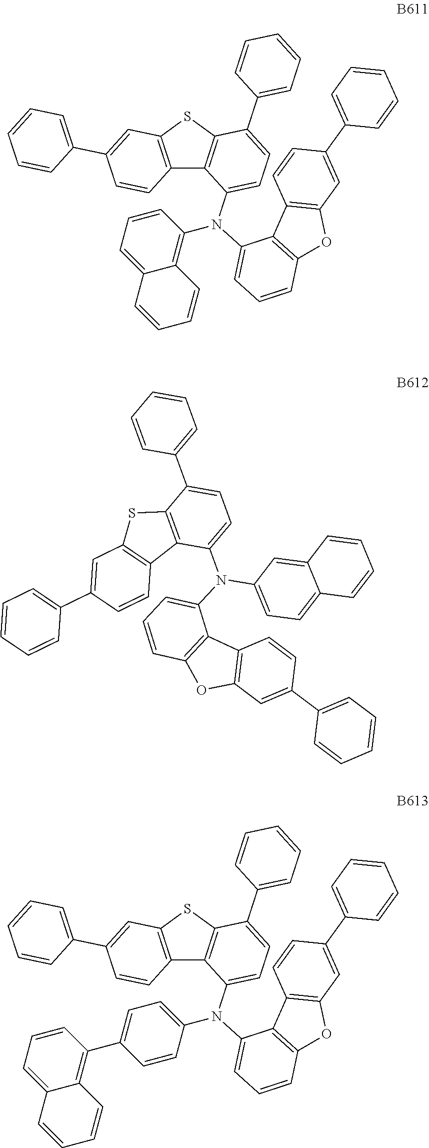 [SMILES: c1ccc(-c2ccc3c(c2)oc2cccc(N(c4cccc5ccccc45)c4ccc(-c5ccccc5)c5sc6cc(-c7ccccc7)ccc6c45)c23)cc1 ; c1ccc(-c2ccc3c(c2)oc2cccc(N(c4ccc5ccccc5c4)c4ccc(-c5ccccc5)c5sc6cc(-c7ccccc7)ccc6c45)c23)cc1 ; c1ccc(-c2ccc3c(c2)oc2cccc(N(c4ccc(-c5cccc6ccccc56)cc4)c4ccc(-c5ccccc5)c5sc6cc(-c7ccccc7)ccc6c45)c23)cc1]

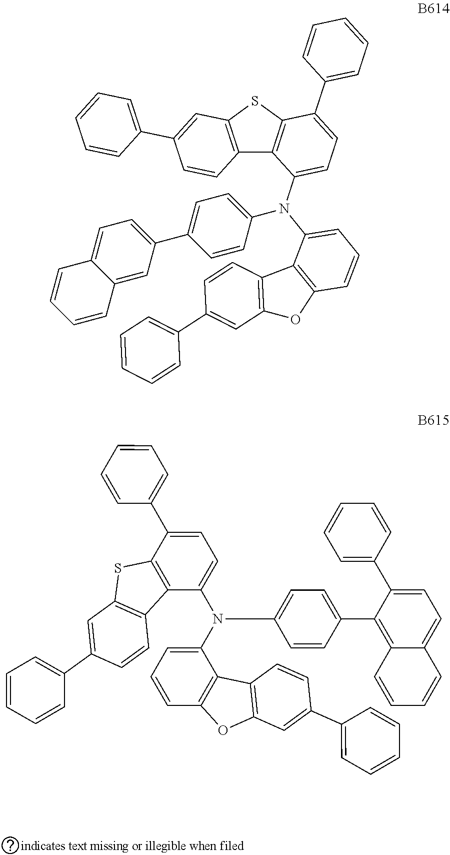 [SMILES: c1ccc(-c2ccc3c(c2)oc2cccc(N(c4ccc(-c5ccc6ccccc6c5)cc4)c4ccc(-c5ccccc5)c5sc6cc(-c7ccccc7)ccc6c45)c23)cc1 ; c1ccc(-c2ccc3c(c2)oc2cccc(N(c4ccc(-c5c(-c6ccccc6)ccc6ccccc56)cc4)c4ccc(-c5ccccc5)c5sc6cc(-c7ccccc7)ccc6c45)c23)cc1]

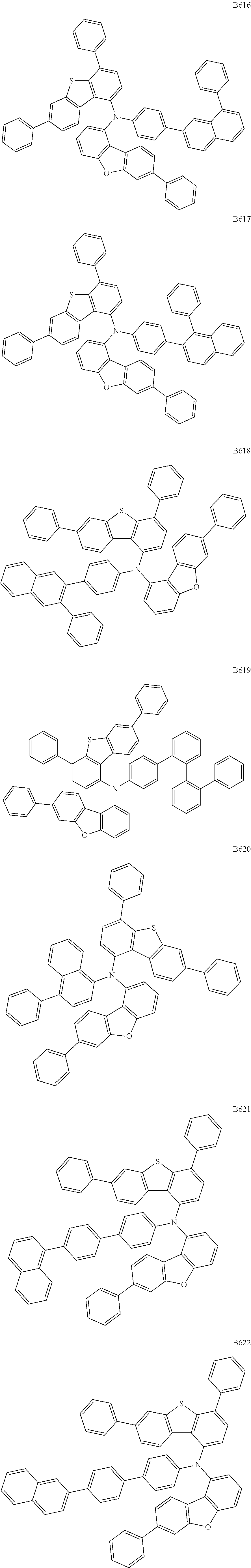

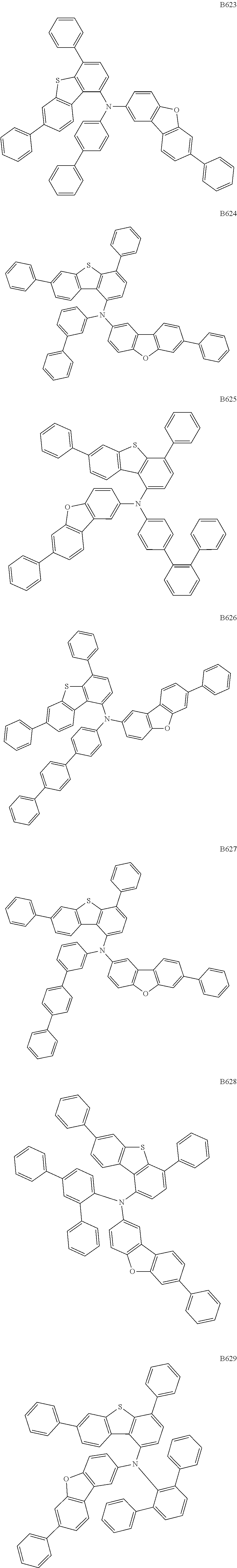

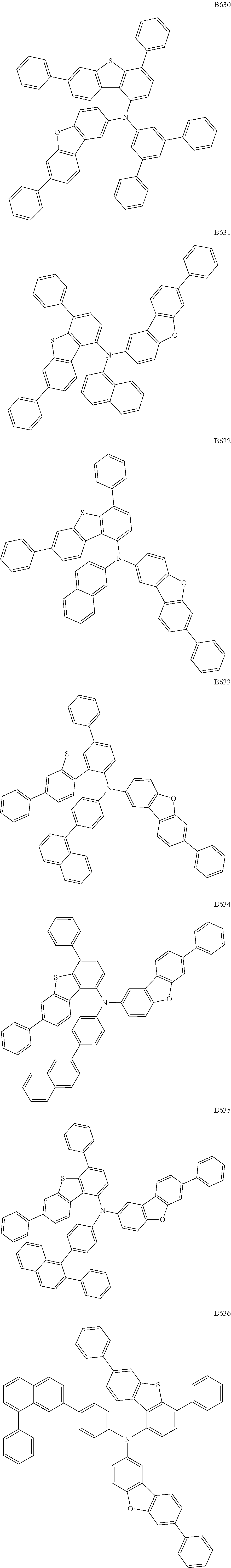

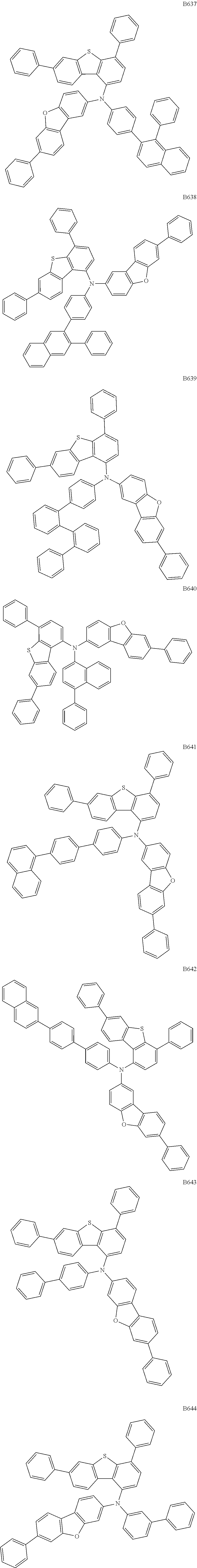

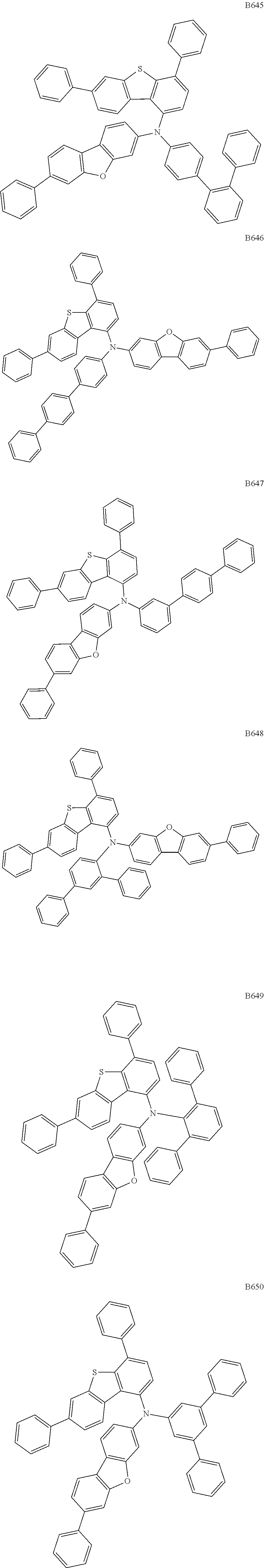

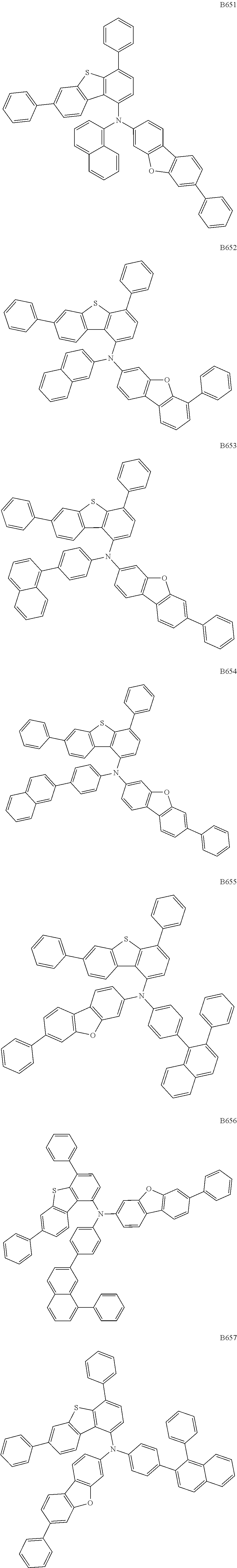

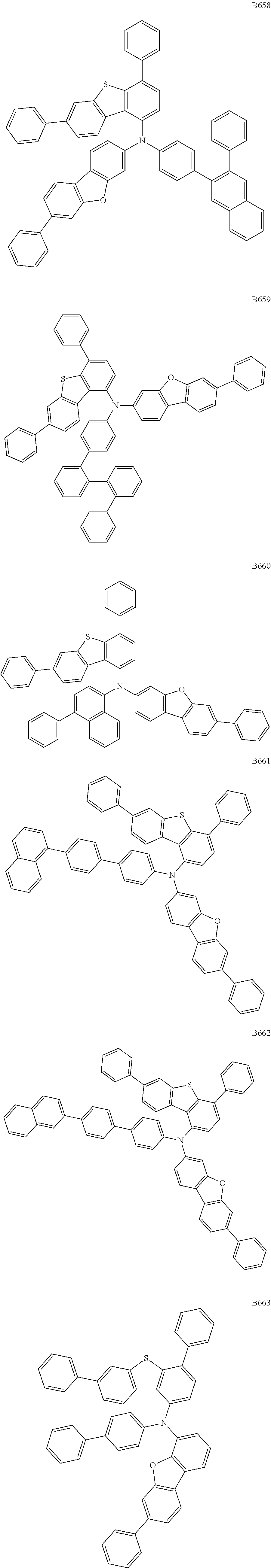

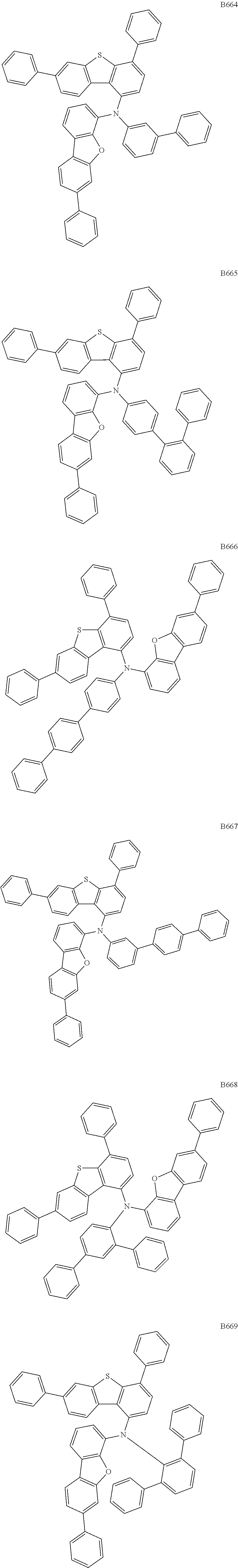

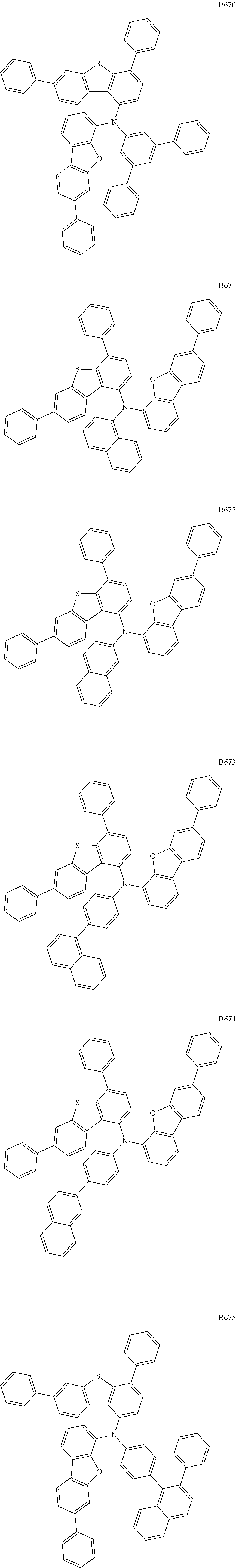

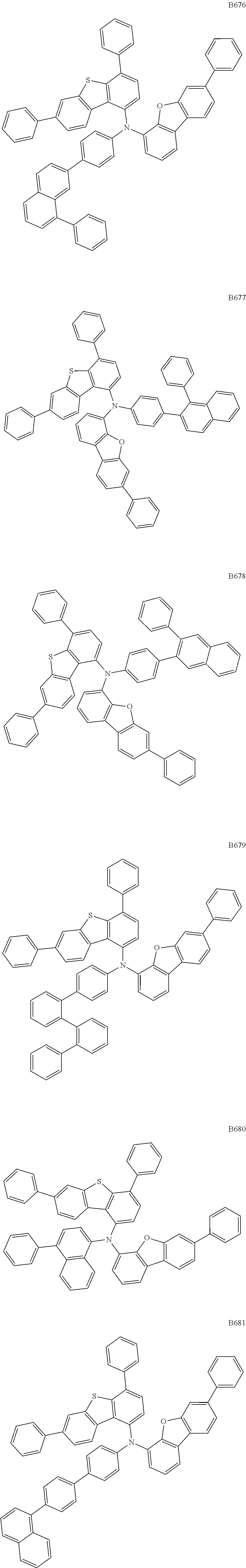

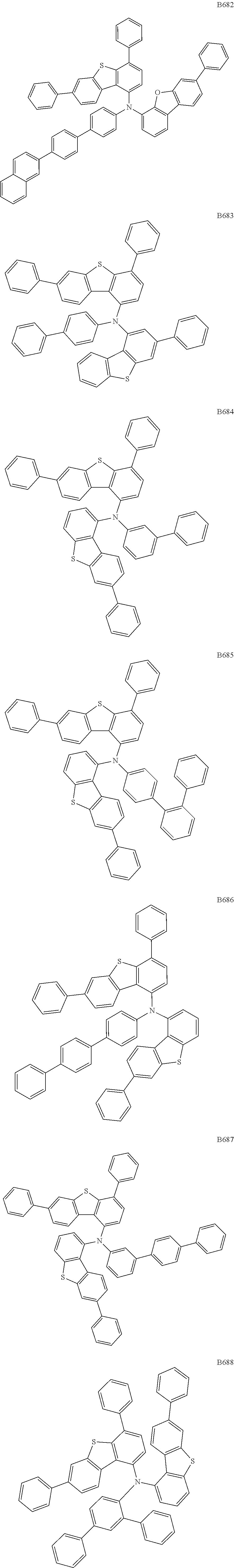

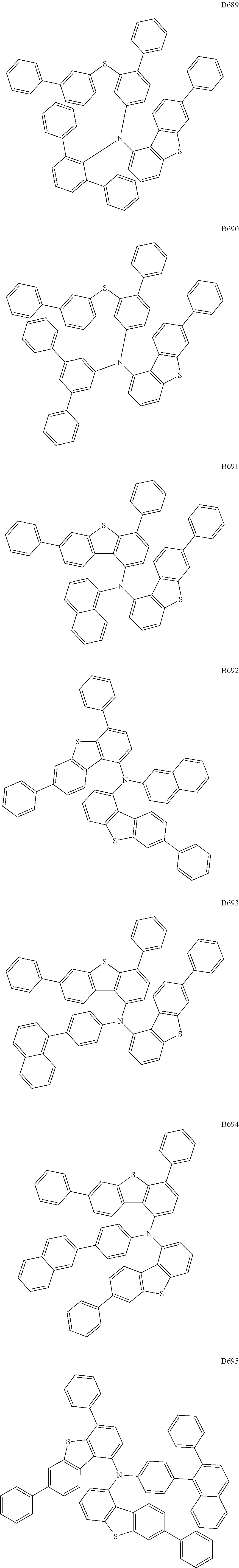

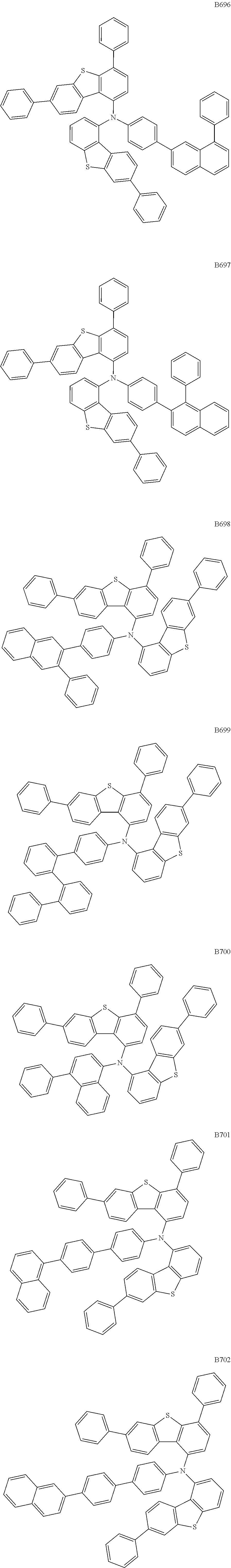

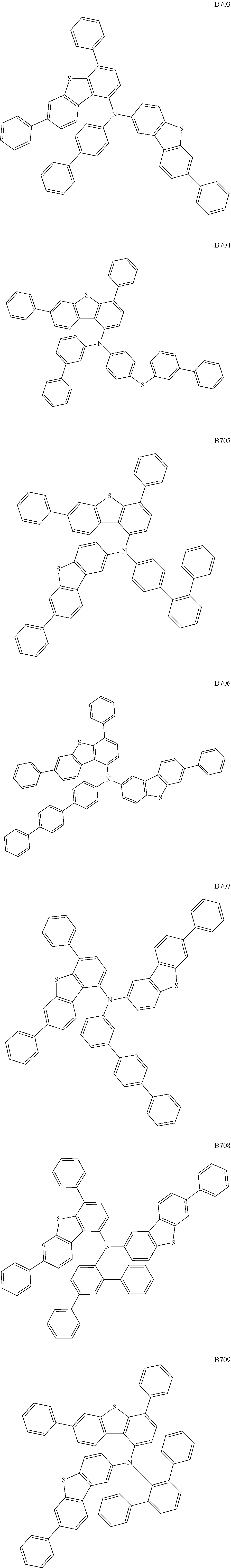

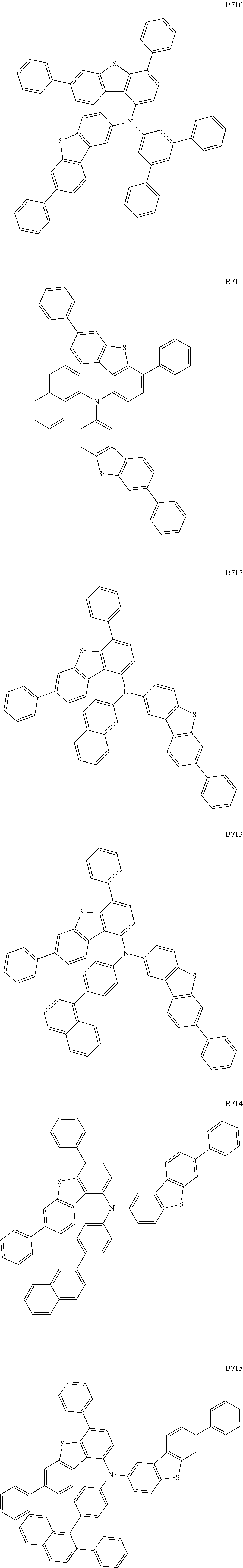

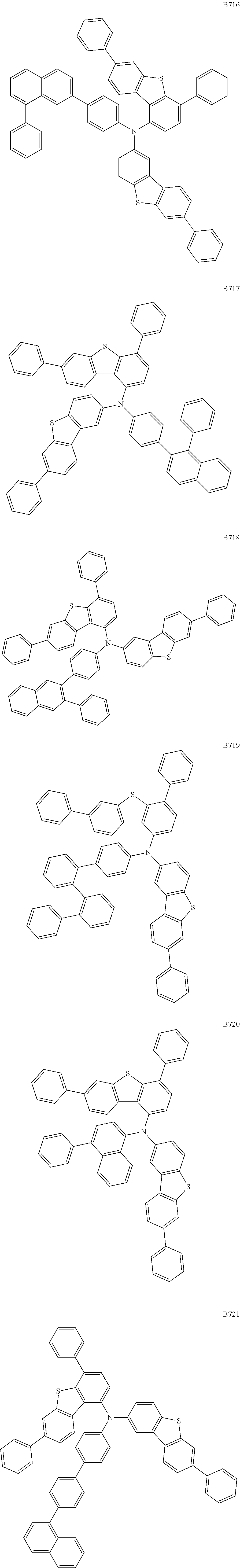

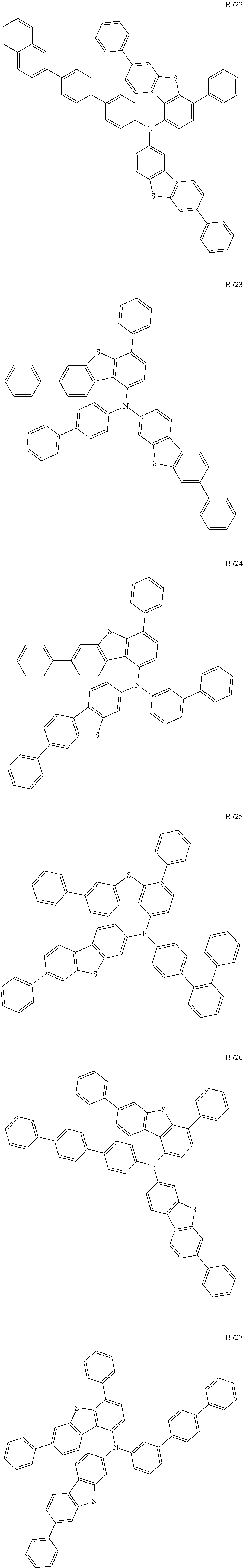

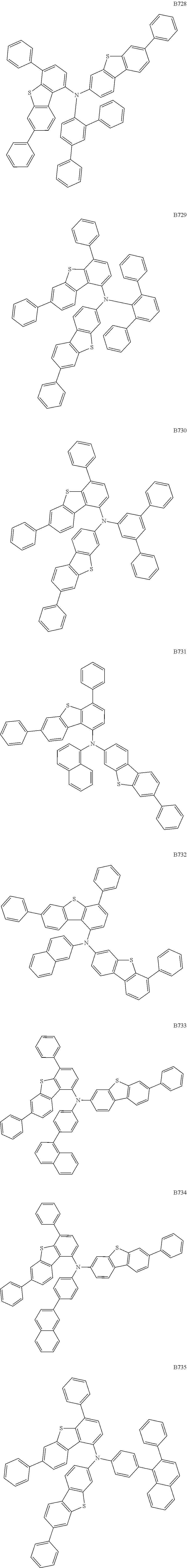

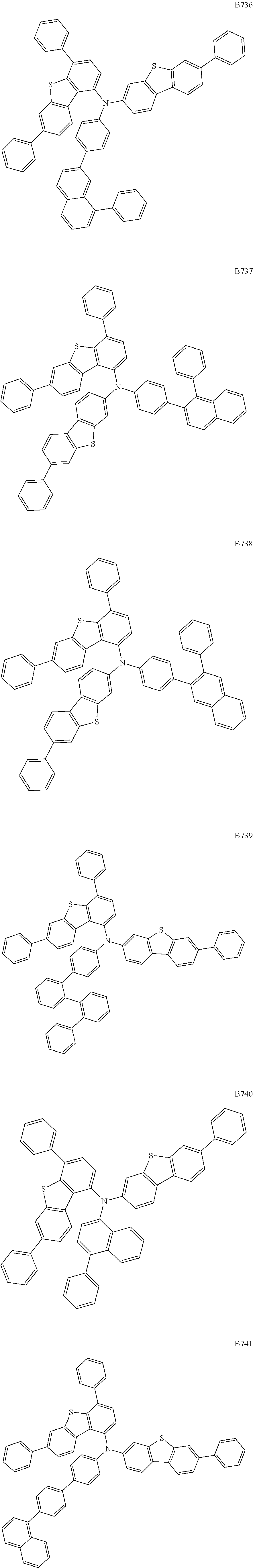

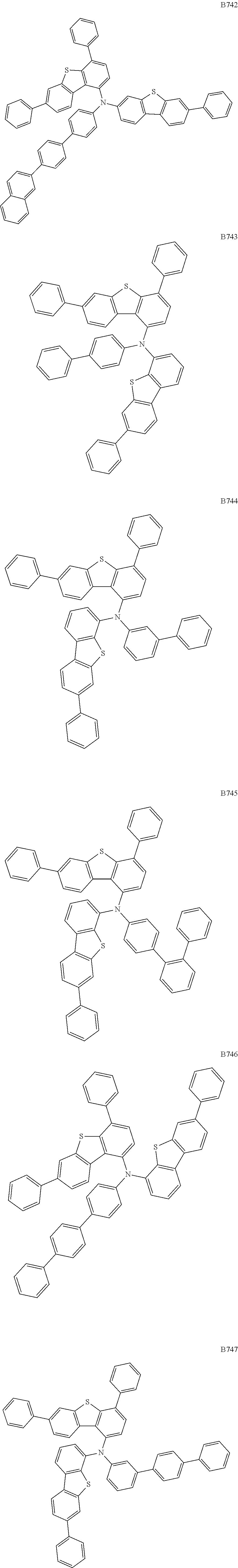

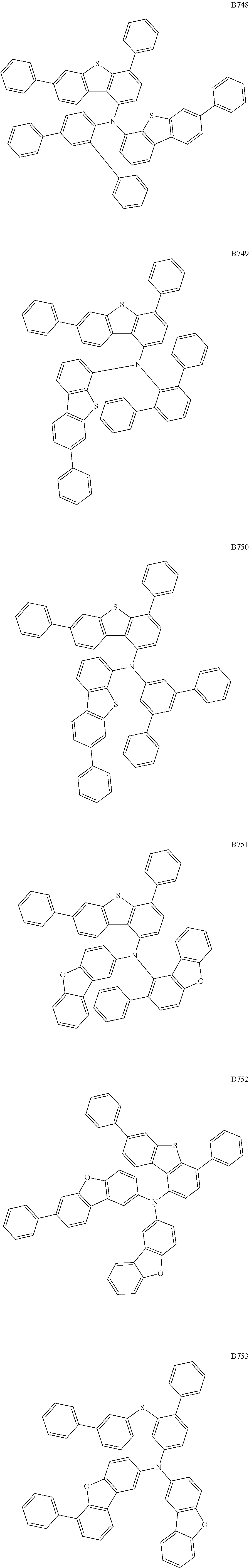

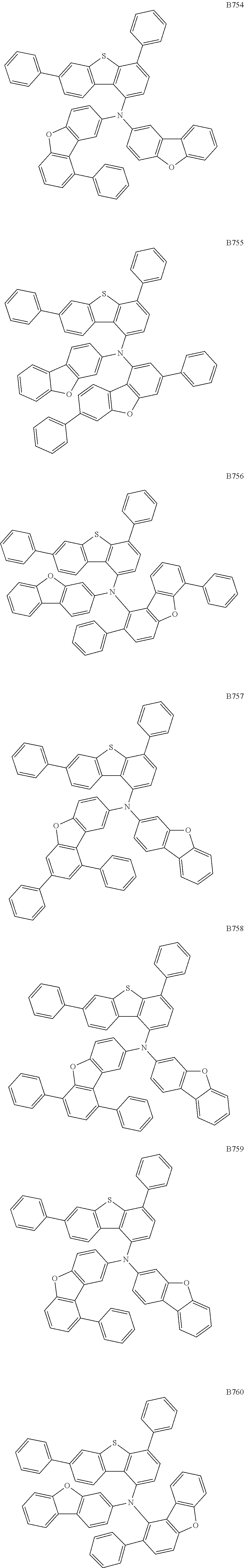

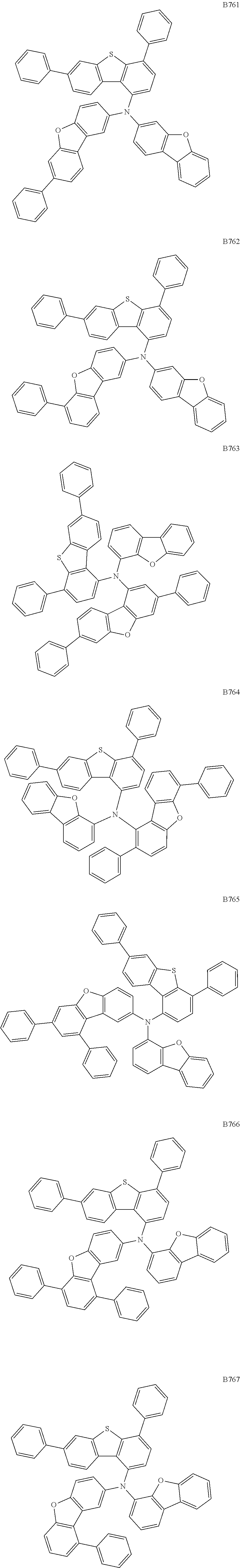

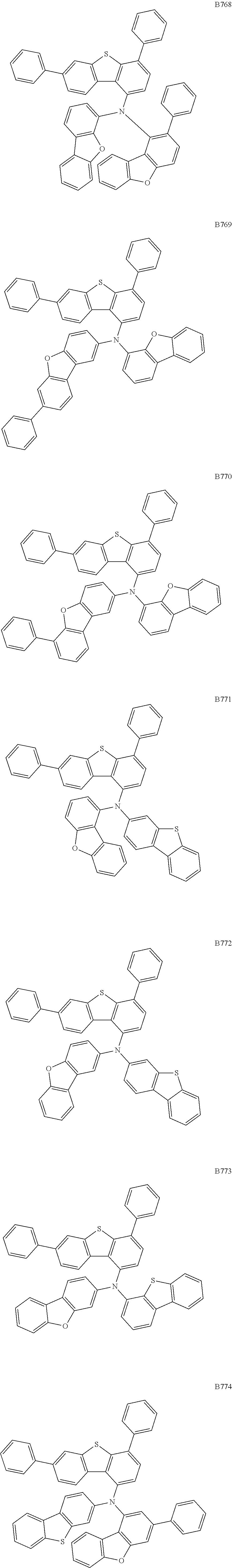

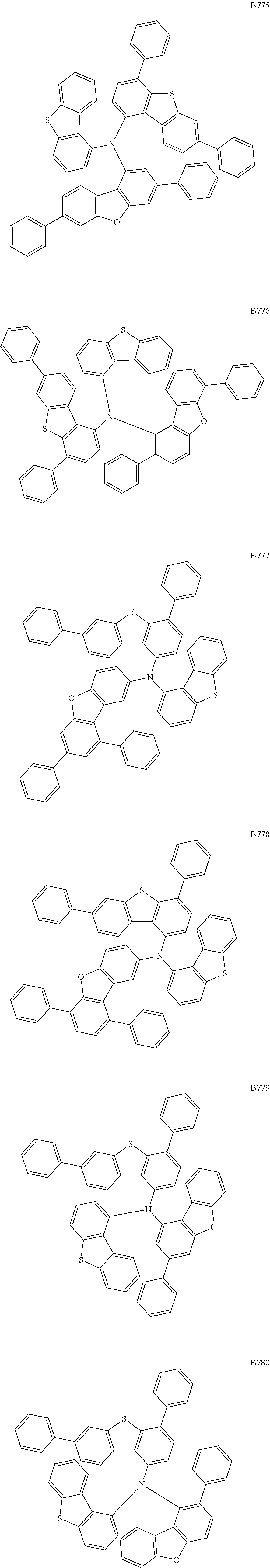

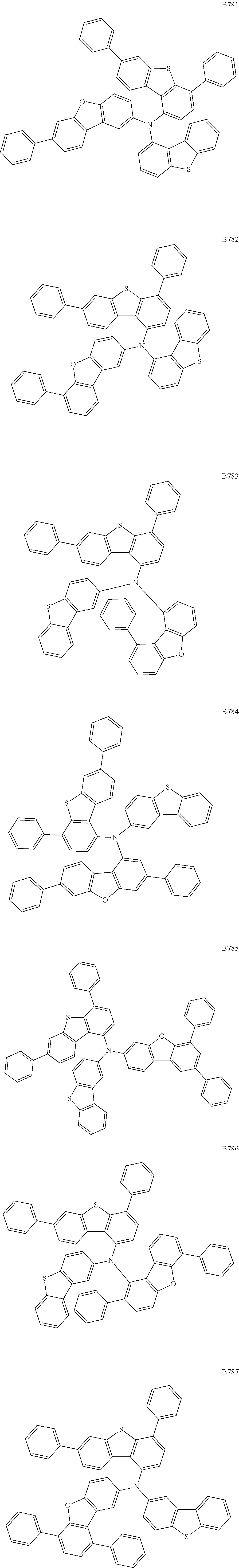

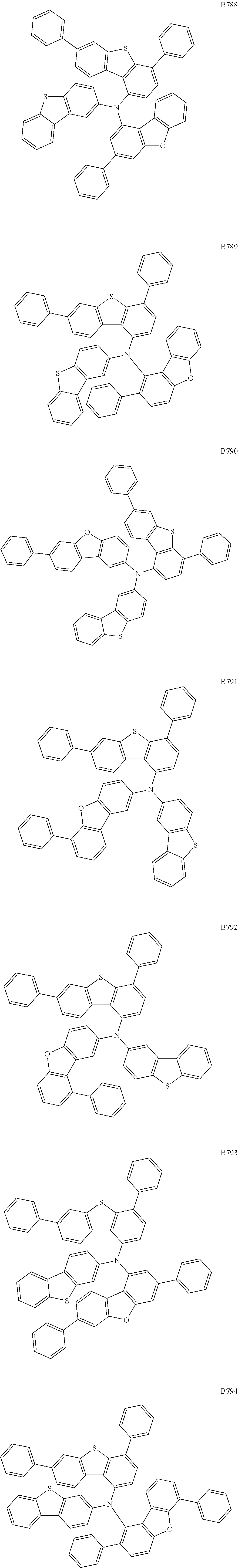

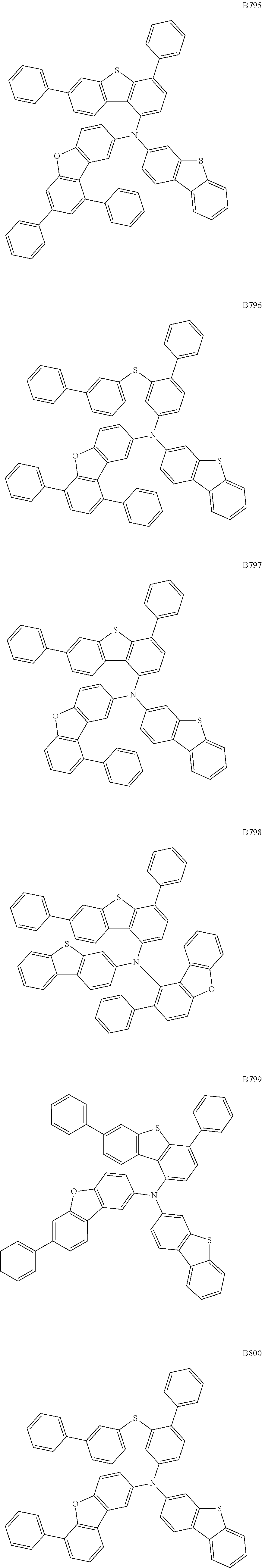

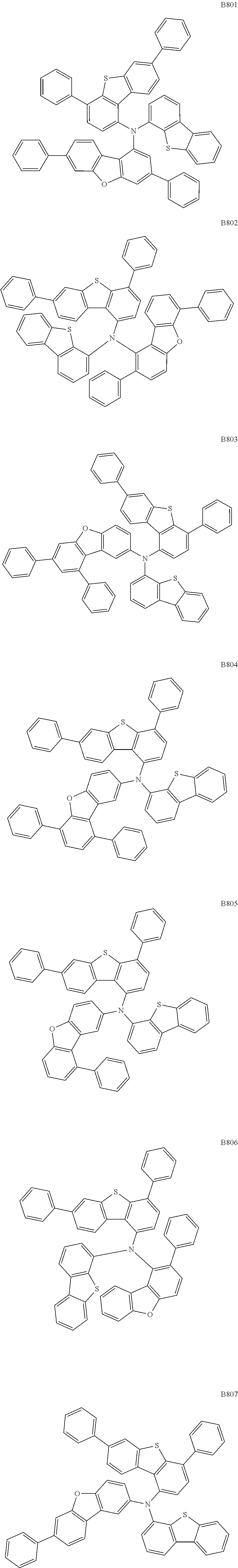

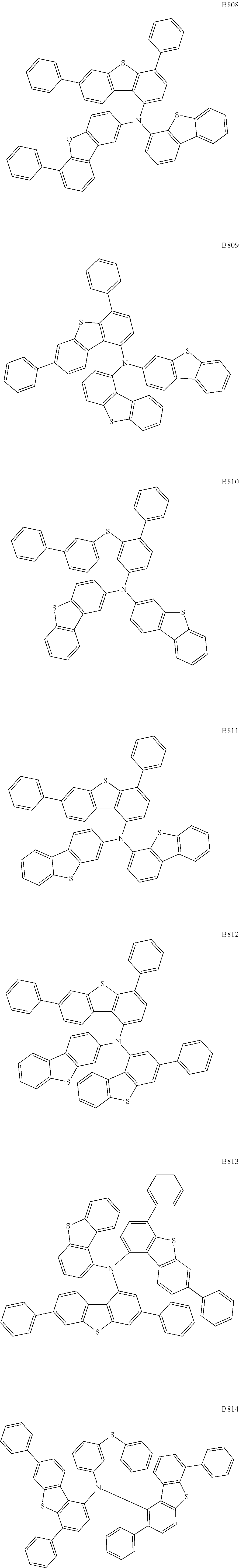

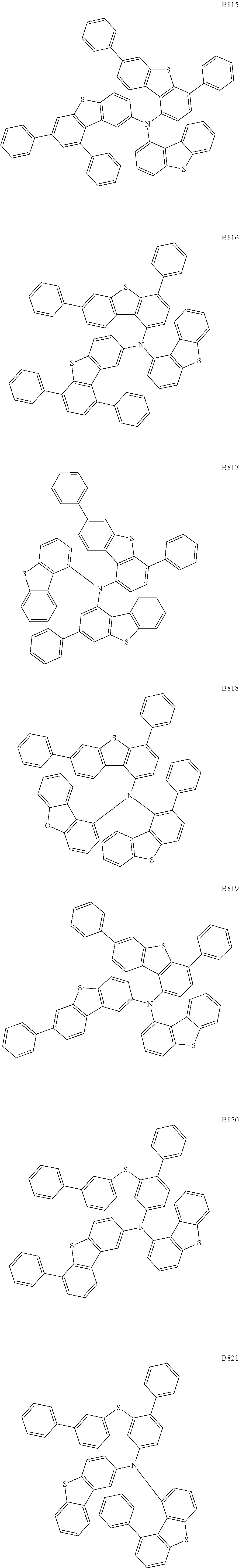

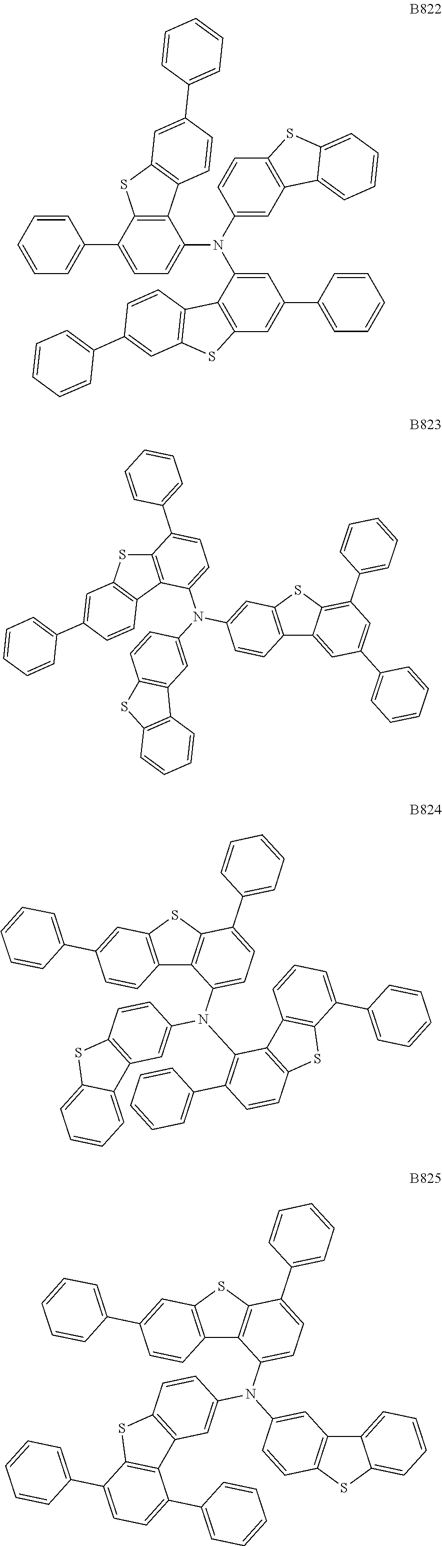 [SMILES: c1ccc(-c2ccc3c(c2)sc2cc(-c4ccccc4)cc(N(c4ccc5sc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4c(-c6ccccc6)cc(-c6ccccc6)cc45)c4ccc5sc6ccccc6c5c4)c23)cc1 ; c1ccc(-c2ccc3c(c2)sc2c(-c4ccccc4)ccc(N(c4ccc5sc6ccccc6c5c4)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6ccccc6c5c4)c4ccc5sc6c(-c7ccccc7)ccc(-c7ccccc7)c6c5c4)c23)cc1]

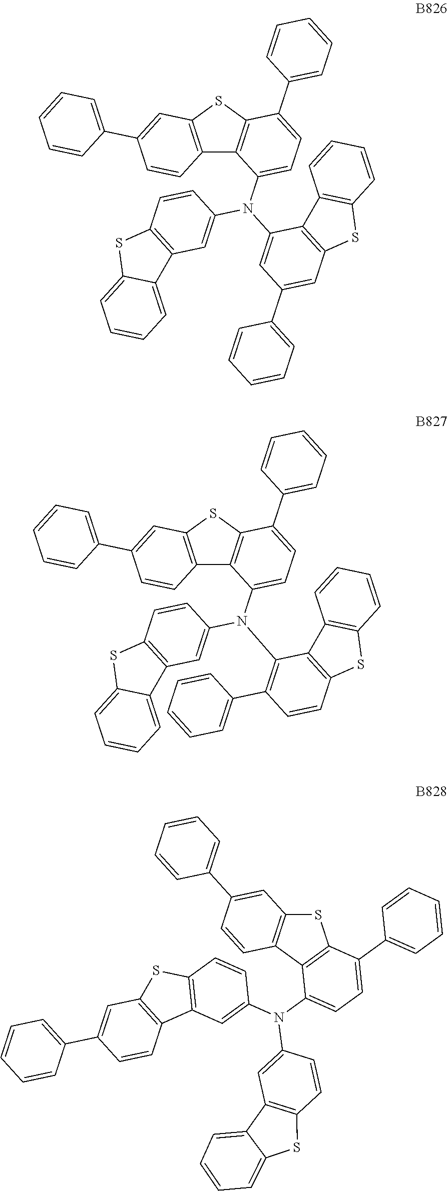 [SMILES: c1ccc(-c2ccc3c(c2)sc2c(-c4ccccc4)ccc(N(c4ccc5sc6ccccc6c5c4)c4cc(-c5ccccc5)cc5sc6ccccc6c45)c23)cc1 ; c1ccc(-c2ccc3c(c2)sc2c(-c4ccccc4)ccc(N(c4ccc5sc6ccccc6c5c4)c4c(-c5ccccc5)ccc5sc6ccccc6c45)c23)cc1 ; c1ccc(-c2ccc3c(c2)sc2ccc(N(c4ccc5sc6ccccc6c5c4)c4ccc(-c5ccccc5)c5sc6cc(-c7ccccc7)ccc6c45)cc23)cc1]

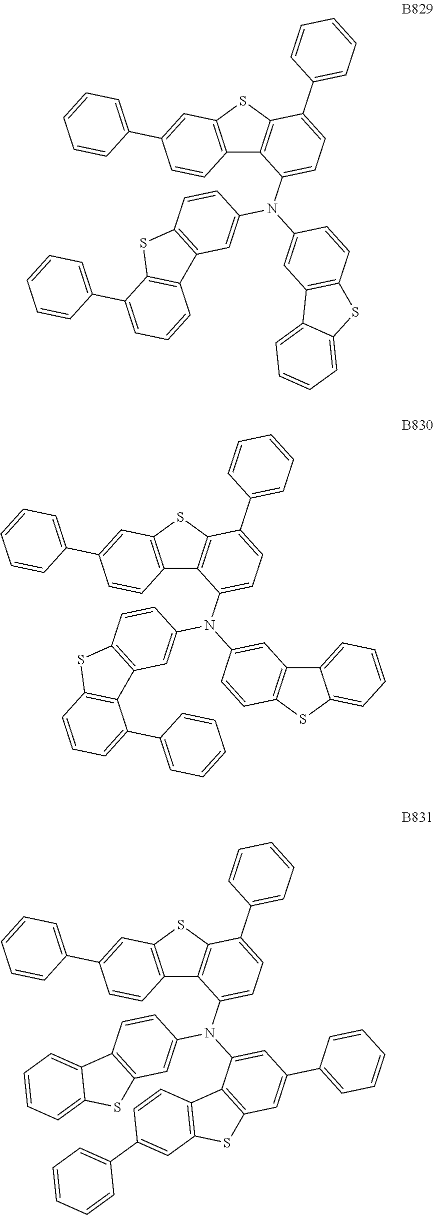 [SMILES: c1ccc(-c2ccc3c(c2)sc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)sc2c(-c4ccccc4)ccc(N(c4ccc5sc6ccccc6c5c4)c4ccc5sc6cccc(-c7ccccc7)c6c5c4)c23)cc1 ; c1ccc(-c2ccc3c(c2)sc2cc(-c4ccccc4)cc(N(c4ccc5c(c4)sc4ccccc45)c4ccc(-c5ccccc5)c5sc6cc(-c7ccccc7)ccc6c45)c23)cc1]

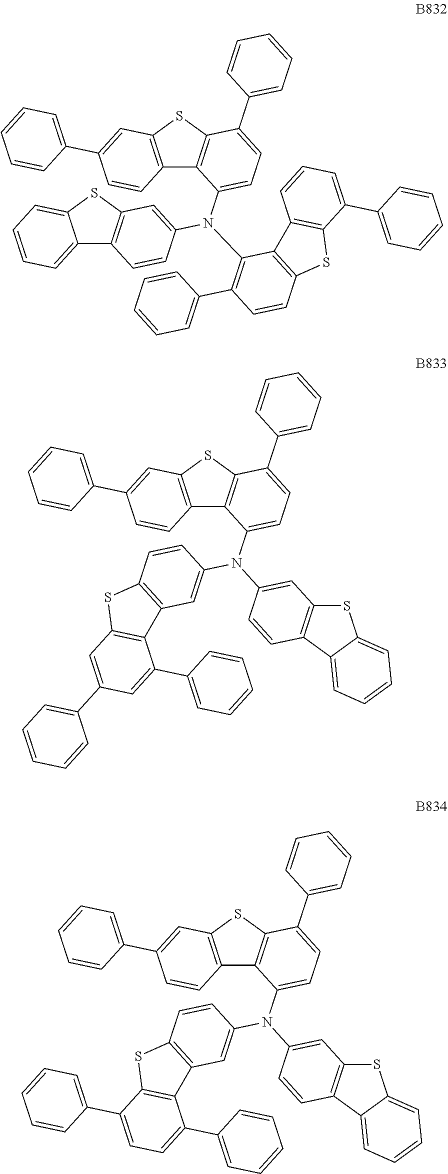 [SMILES: c1ccc(-c2ccc3c(c2)sc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5c(c4)sc4ccccc45)c4ccc5sc6cc(-c7ccccc7)cc(-c7ccccc7)c6c5c4)c23)cc1 ; c1ccc(-c2ccc3c(c2)sc2c(-c4ccccc4)ccc(N(c4ccc5c(c4)sc4ccccc45)c4ccc5sc6c(-c7ccccc7)ccc(-c7ccccc7)c6c5c4)c23)cc1]

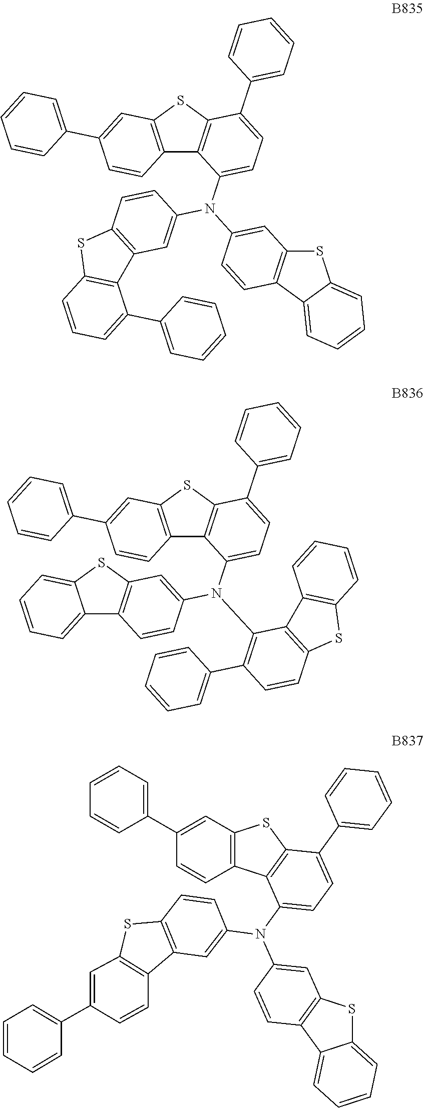 [SMILES: c1ccc(-c2ccc3c(c2)sc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)sc2c(-c4ccccc4)ccc(N(c4ccc5c(c4)sc4ccccc45)c4c(-c5ccccc5)ccc5sc6ccccc6c45)c23)cc1 ; c1ccc(-c2ccc3c(c2)sc2ccc(N(c4ccc5c(c4)sc4ccccc45)c4ccc(-c5ccccc5)c5sc6cc(-c7ccccc7)ccc6c45)cc23)cc1]

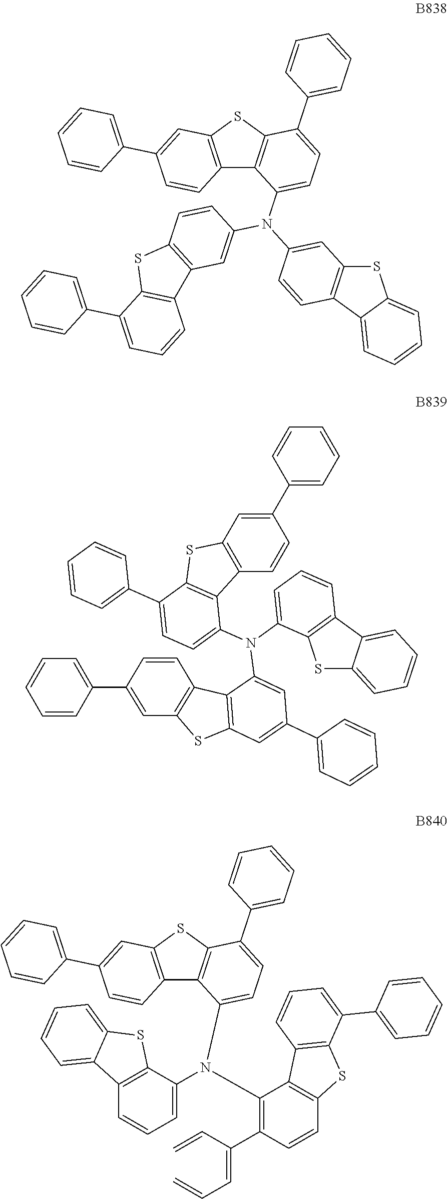 [SMILES: c1ccc(-c2ccc3c(c2)sc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)sc2cc(-c4ccccc4)cc(N(c4cccc5c4sc4ccccc45)c4ccc(-c5ccccc5)c5sc6cc(-c7ccccc7)ccc6c45)c23)cc1 ; C=C/C=C(\C=C)c1ccc2sc3c(-c4ccccc4)cccc3c2c1N(c1cccc2c1sc1ccccc12)c1ccc(-c2ccccc2)c2sc3cc(-c4ccccc4)ccc3c12]

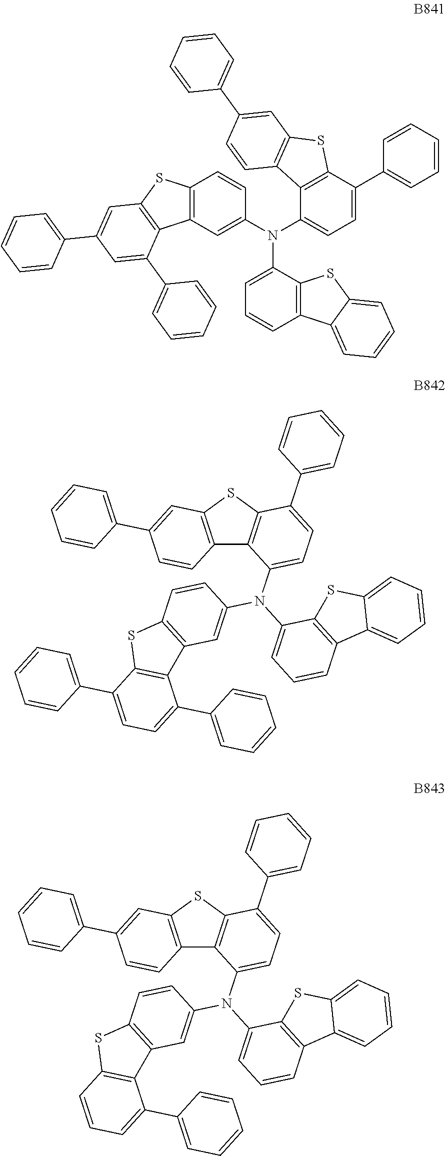 [SMILES: c1ccc(-c2ccc3c(c2)sc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5c4sc4ccccc45)c23)cc1 ; c1ccc(-c2ccc3c(c2)sc2c(-c4ccccc4)ccc(N(c4ccc5sc6cccc(-c7ccccc7)c6c5c4)c4cccc5c4sc4ccccc45)c23)cc1]

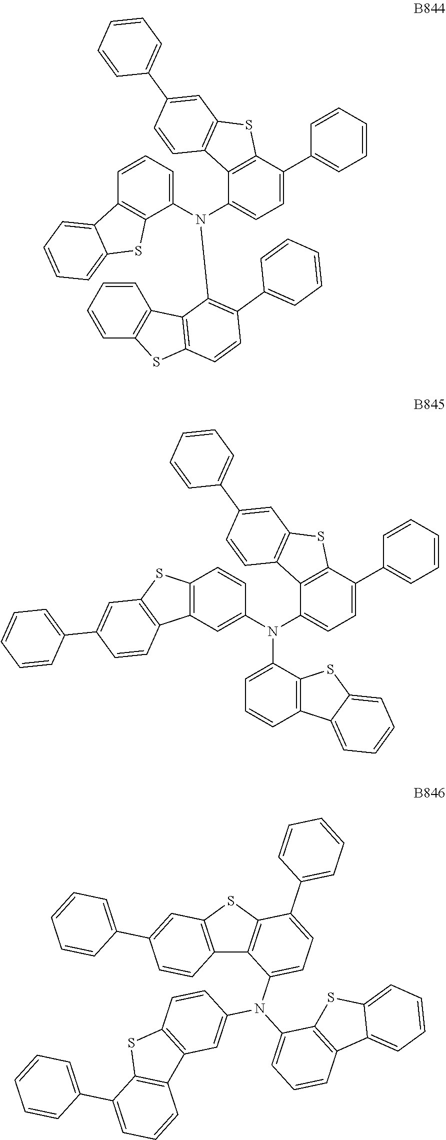 [SMILES: c1ccc(-c2ccc3c(c2)sc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)sc2ccc(N(c4cccc5c4sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5c4sc4ccccc45)c23)cc1]

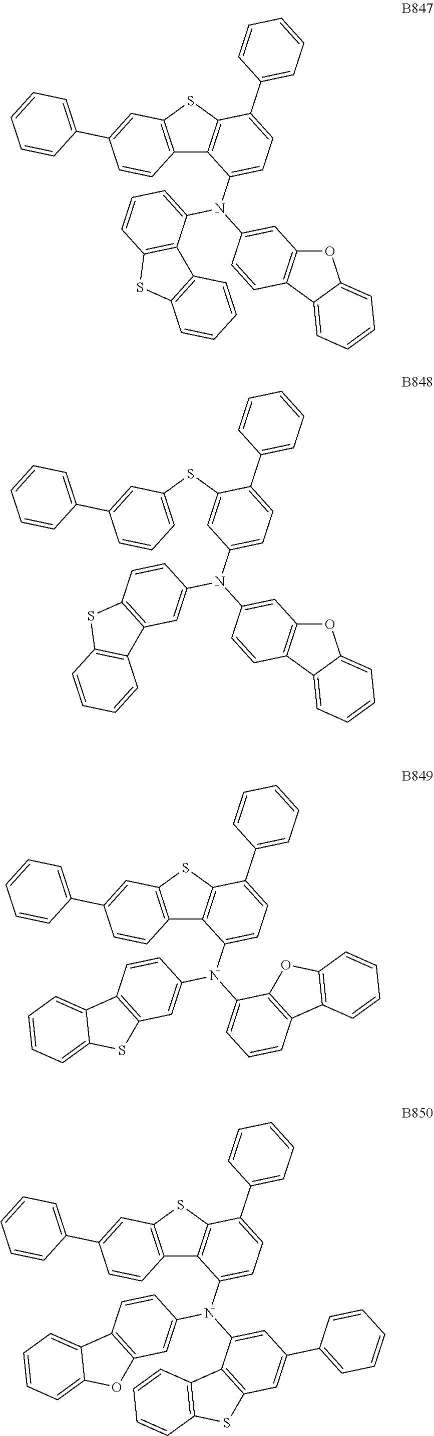 [SMILES: c1ccc(-c2ccc3c(c2)sc2c(-c4ccccc4)ccc(N(c4ccc5c(c4)oc4ccccc45)c4cccc5sc6ccccc6c45)c23)cc1 ; c1ccc(-c2cccc(Sc3cc(N(c4ccc5c(c4)oc4ccccc45)c4ccc5sc6ccccc6c5c4)ccc3-c3ccccc3)c2)cc1 ; c1ccc(-c2ccc3c(c2)sc2c(-c4ccccc4)ccc(N(c4ccc5c(c4)sc4ccccc45)c4cccc5c4oc4ccccc45)c23)cc1 ; c1ccc(-c2ccc3c(c2)sc2c(-c4ccccc4)ccc(N(c4ccc5c(c4)oc4ccccc45)c4cc(-c5ccccc5)cc5sc6ccccc6c45)c23)cc1]

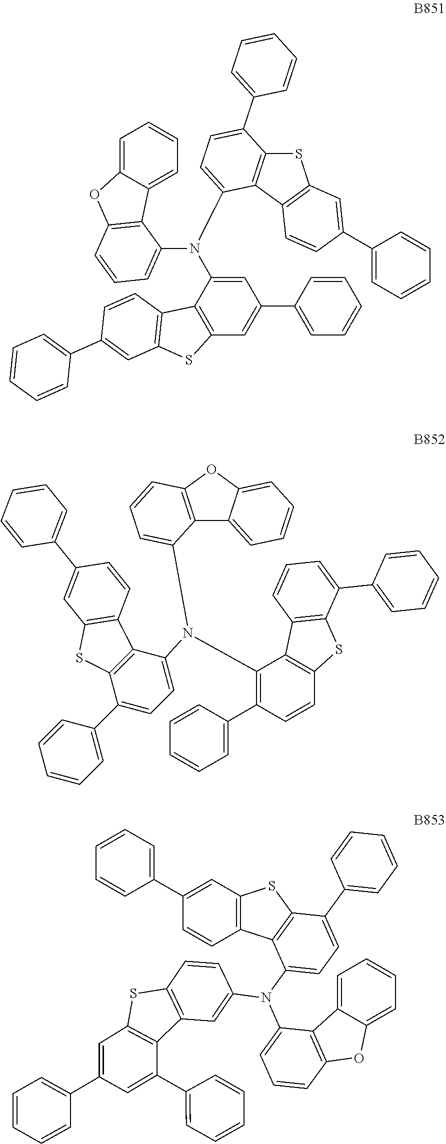 [SMILES: c1ccc(-c2ccc3c(c2)sc2cc(-c4ccccc4)cc(N(c4cccc5oc6ccccc6c45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5oc6ccccc6c45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5oc6ccccc6c45)c23)cc1]

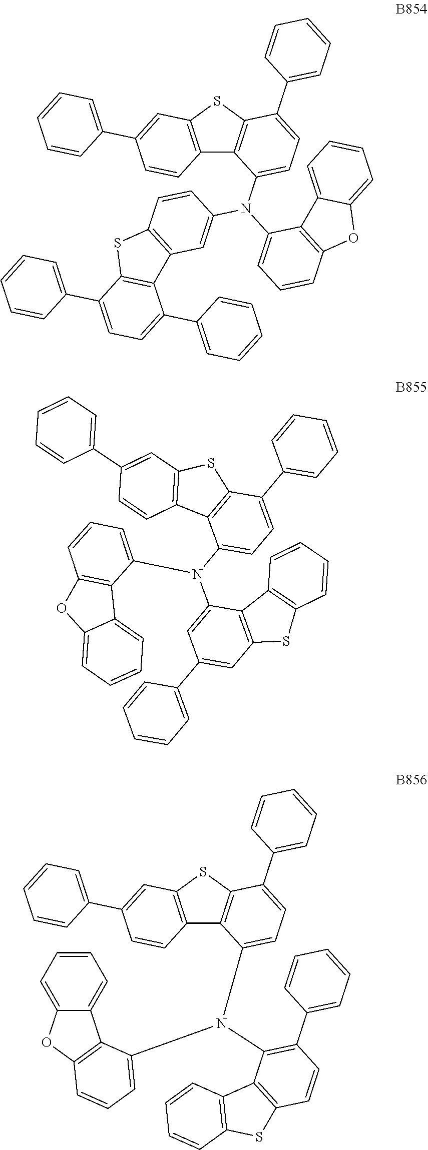 [SMILES: c1ccc(-c2ccc3c(c2)sc2c(-c4ccccc4)ccc(N(c4ccc5sc6c(-c7ccccc7)ccc(-c7ccccc7)c6c5c4)c4cccc5oc6ccccc6c45)c23)cc1 ; c1ccc(-c2ccc3c(c2)sc2c(-c4ccccc4)ccc(N(c4cccc5oc6ccccc6c45)c4cc(-c5ccccc5)cc5sc6ccccc6c45)c23)cc1 ; c1ccc(-c2ccc3c(c2)sc2c(-c4ccccc4)ccc(N(c4cccc5oc6ccccc6c45)c4c(-c5ccccc5)ccc5sc6ccccc6c45)c23)cc1]

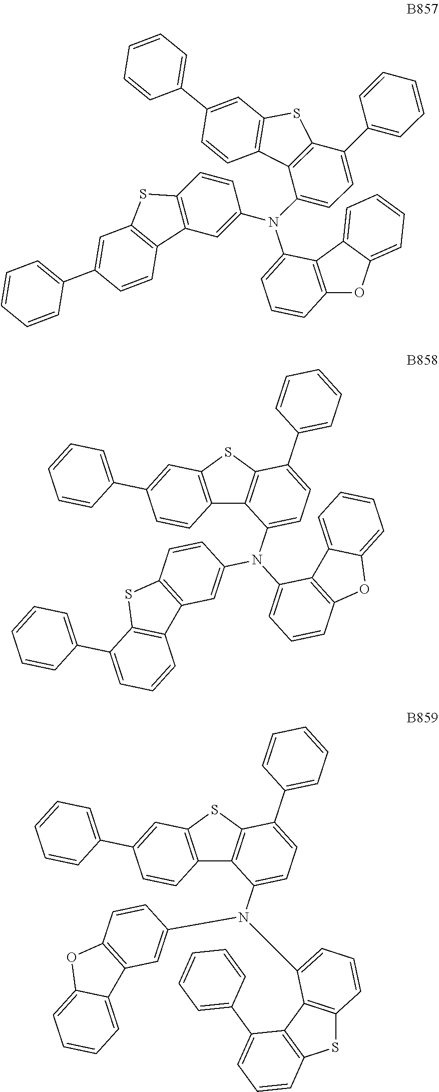 [SMILES: c1ccc(-c2ccc3c(c2)sc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4cccc5sc6cccc(-c7ccccc7)c6c45)c23)cc1]

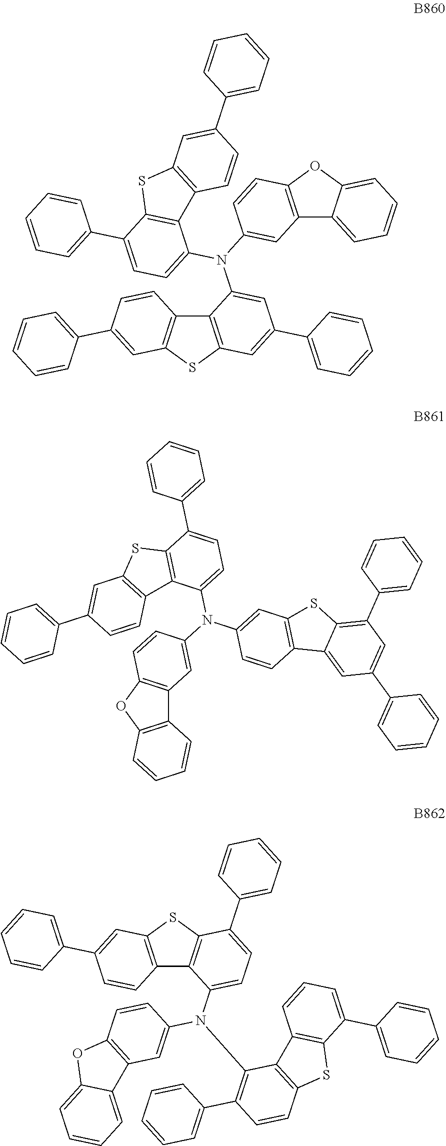 [SMILES: c1ccc(-c2ccc3c(c2)sc2cc(-c4ccccc4)cc(N(c4ccc5oc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4c(-c6ccccc6)cc(-c6ccccc6)cc45)c4ccc5oc6ccccc6c5c4)c23)cc1 ; c1ccc(-c2ccc3c(c2)sc2c(-c4ccccc4)ccc(N(c4ccc5oc6ccccc6c5c4)c4c(-c5ccccc5)ccc5sc6c(-c7ccccc7)cccc6c45)c23)cc1]

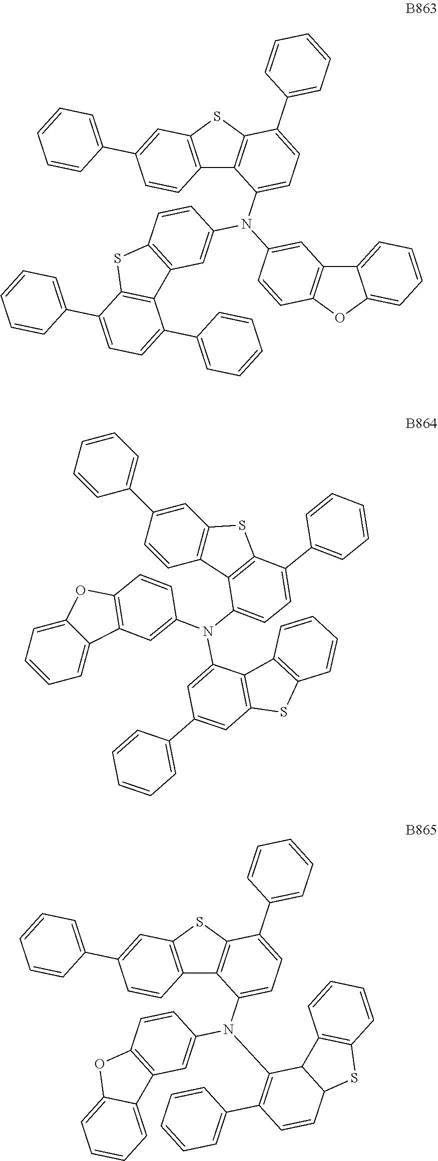 [SMILES: c1ccc(-c2ccc3c(c2)sc2c(-c4ccccc4)ccc(N(c4ccc5oc6ccccc6c5c4)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5oc6ccccc6c5c4)c4cc(-c5ccccc5)cc5sc6ccccc6c45)c23)cc1 ; C1=CC2Sc3ccccc3C2C(N(c2ccc3oc4ccccc4c3c2)c2ccc(-c3ccccc3)c3sc4cc(-c5ccccc5)ccc4c23)=C1c1ccccc1]

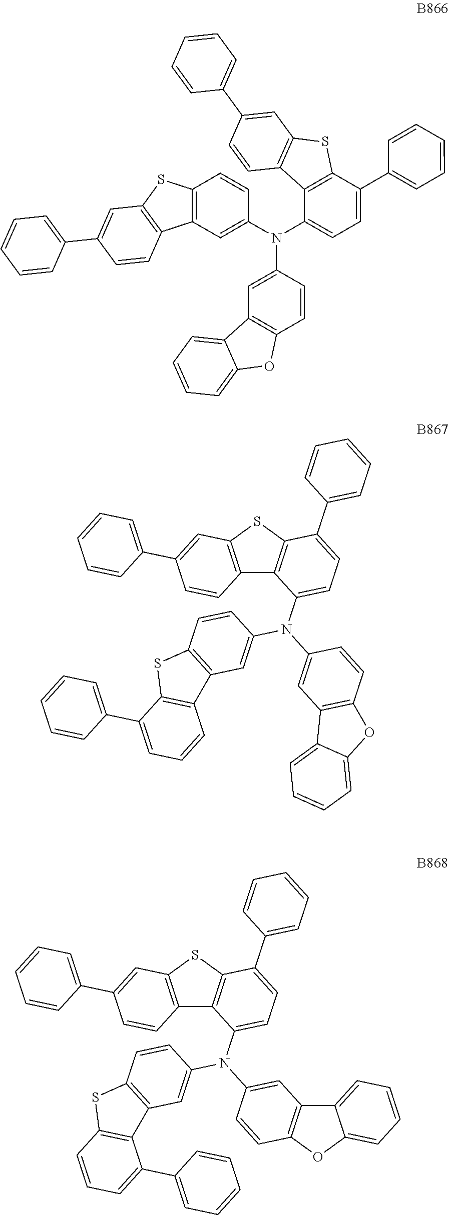 [SMILES: c1ccc(-c2ccc3c(c2)sc2ccc(N(c4ccc5oc6ccccc6c5c4)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6ccccc6c5c4)c4ccc5sc6c(-c7ccccc7)cccc6c5c4)c23)cc1 ; c1ccc(-c2ccc3c(c2)sc2c(-c4ccccc4)ccc(N(c4ccc5oc6ccccc6c5c4)c4ccc5sc6cccc(-c7ccccc7)c6c5c4)c23)cc1]

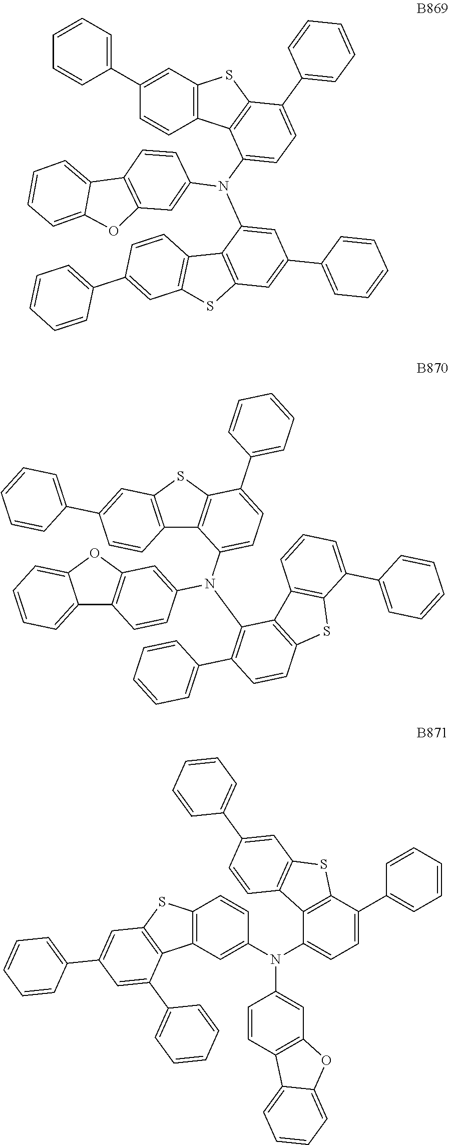 [SMILES: c1ccc(-c2ccc3c(c2)sc2cc(-c4ccccc4)cc(N(c4ccc5c(c4)oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)oc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5c(c4)oc4ccccc45)c4ccc5sc6cc(-c7ccccc7)cc(-c7ccccc7)c6c5c4)c23)cc1]

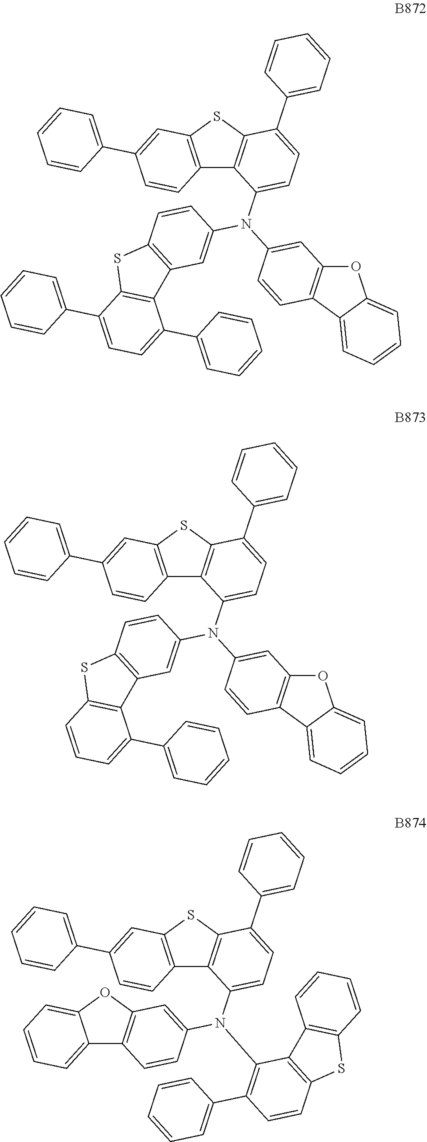 [SMILES: c1ccc(-c2ccc3c(c2)sc2c(-c4ccccc4)ccc(N(c4ccc5c(c4)oc4ccccc45)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5c(c4)oc4ccccc45)c4ccc5sc6cccc(-c7ccccc7)c6c5c4)c23)cc1 ; c1ccc(-c2ccc3c(c2)sc2c(-c4ccccc4)ccc(N(c4ccc5c(c4)oc4ccccc45)c4c(-c5ccccc5)ccc5sc6ccccc6c45)c23)cc1]

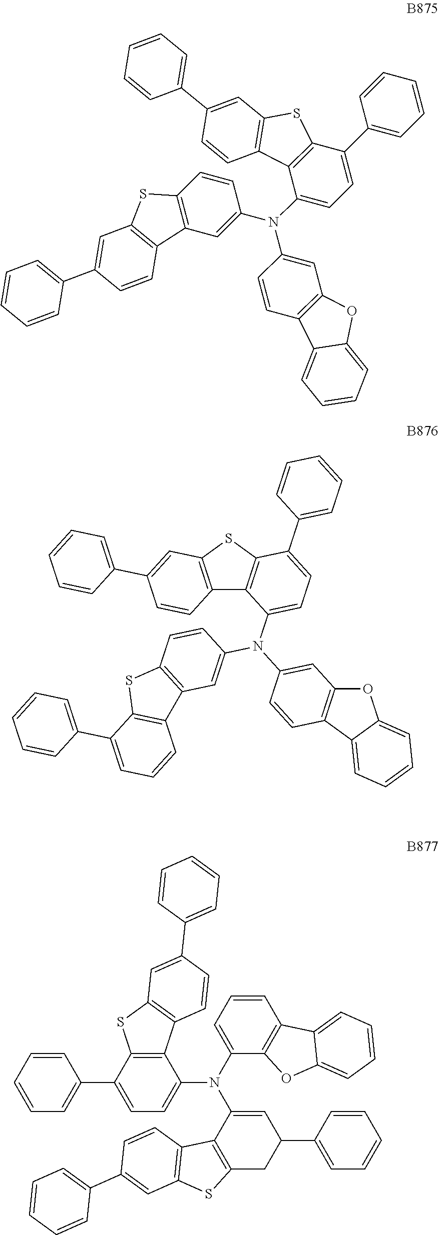 [SMILES: c1ccc(-c2ccc3c(c2)sc2ccc(N(c4ccc5c(c4)oc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5c(c4)oc4ccccc45)c4ccc5sc6c(-c7ccccc7)cccc6c5c4)c23)cc1 ; C1=C(N(c2cccc3c2oc2ccccc23)c2ccc(-c3ccccc3)c3sc4cc(-c5ccccc5)ccc4c23)c2c(sc3cc(-c4ccccc4)ccc23)CC1c1ccccc1]

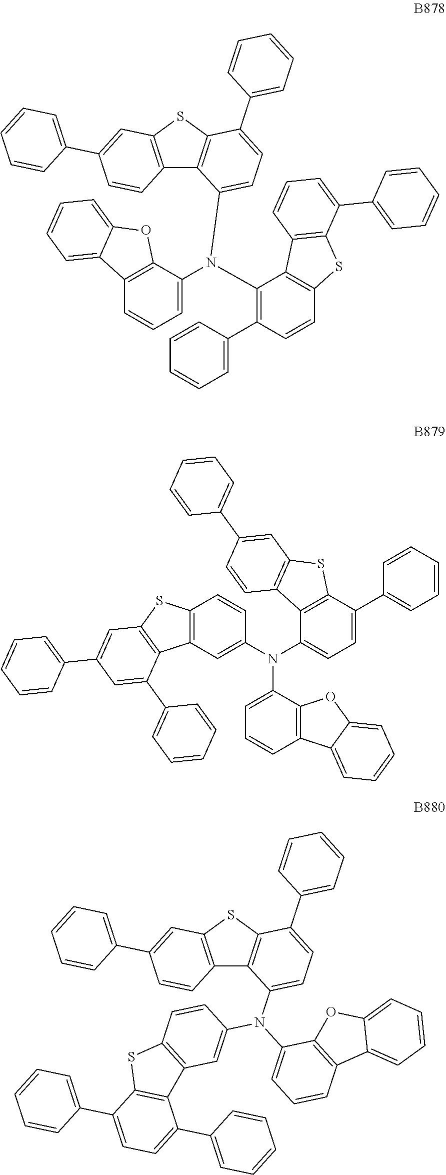 [SMILES: c1ccc(-c2ccc3c(c2)sc2c(-c4ccccc4)ccc(N(c4cccc5c4oc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5c4oc4ccccc45)c23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5c4oc4ccccc45)c23)cc1]

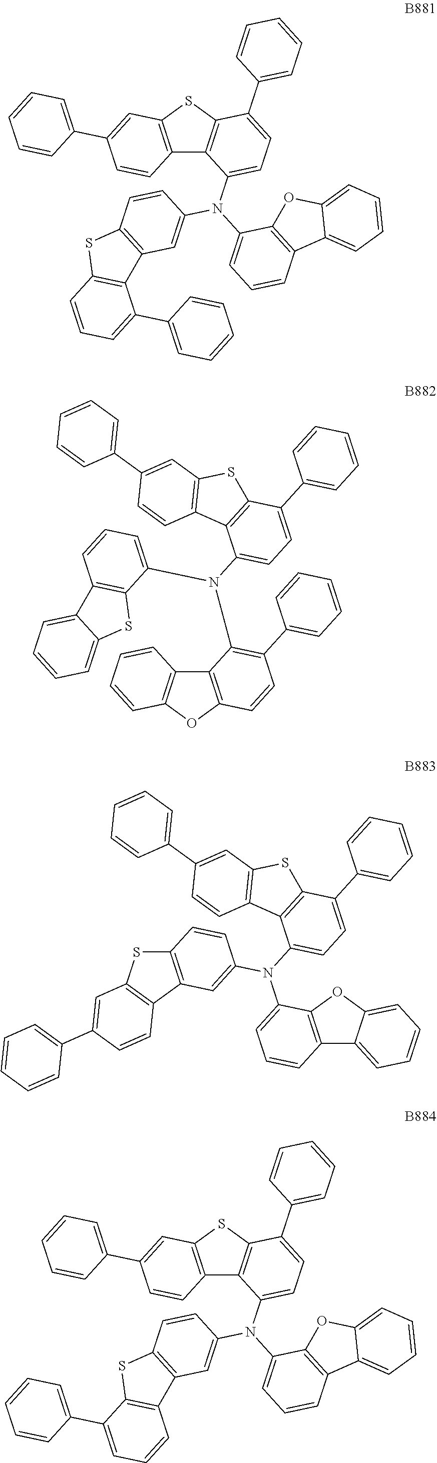 [SMILES: c1ccc(-c2ccc3c(c2)sc2c(-c4ccccc4)ccc(N(c4ccc5sc6cccc(-c7ccccc7)c6c5c4)c4cccc5c4oc4ccccc45)c23)cc1 ; c1ccc(-c2ccc3c(c2)sc2c(-c4ccccc4)ccc(N(c4cccc5c4sc4ccccc45)c4c(-c5ccccc5)ccc5oc6ccccc6c45)c23)cc1 ; c1ccc(-c2ccc3c(c2)sc2ccc(N(c4cccc5c4oc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5c4oc4ccccc45)c23)cc1]

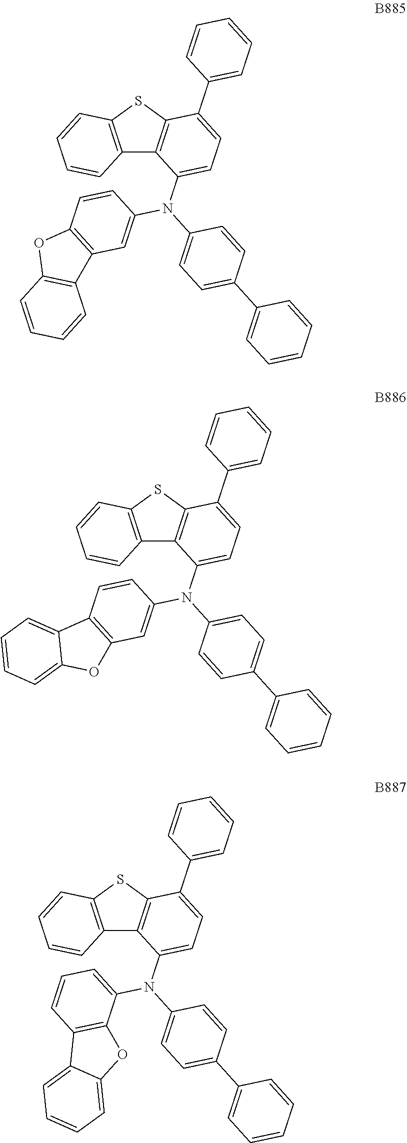 [SMILES: c1ccc(-c2ccc(N(c3ccc4oc5ccccc5c4c3)c3ccc(-c4ccccc4)c4sc5ccccc5c34)cc2)cc1 ; c1ccc(-c2ccc(N(c3ccc4c(c3)oc3ccccc34)c3ccc(-c4ccccc4)c4sc5ccccc5c34)cc2)cc1 ; c1ccc(-c2ccc(N(c3cccc4c3oc3ccccc34)c3ccc(-c4ccccc4)c4sc5ccccc5c34)cc2)cc1]

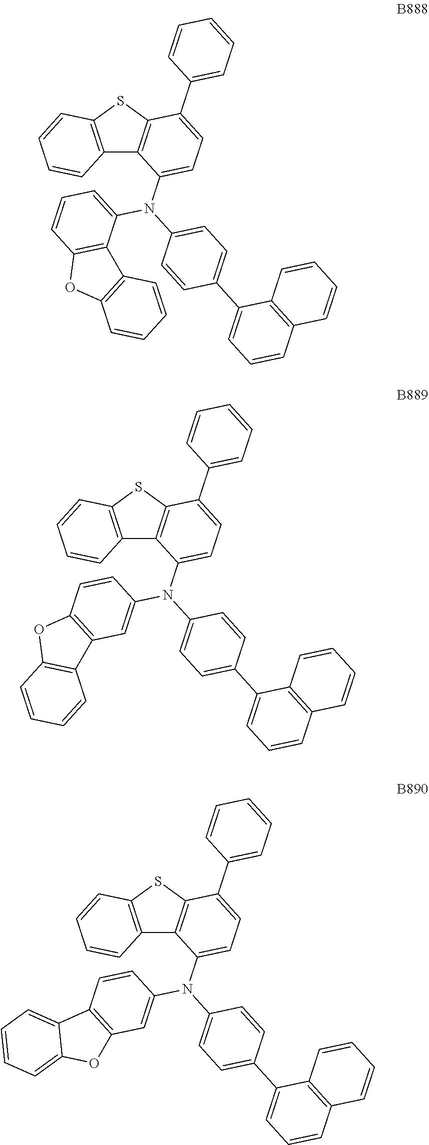

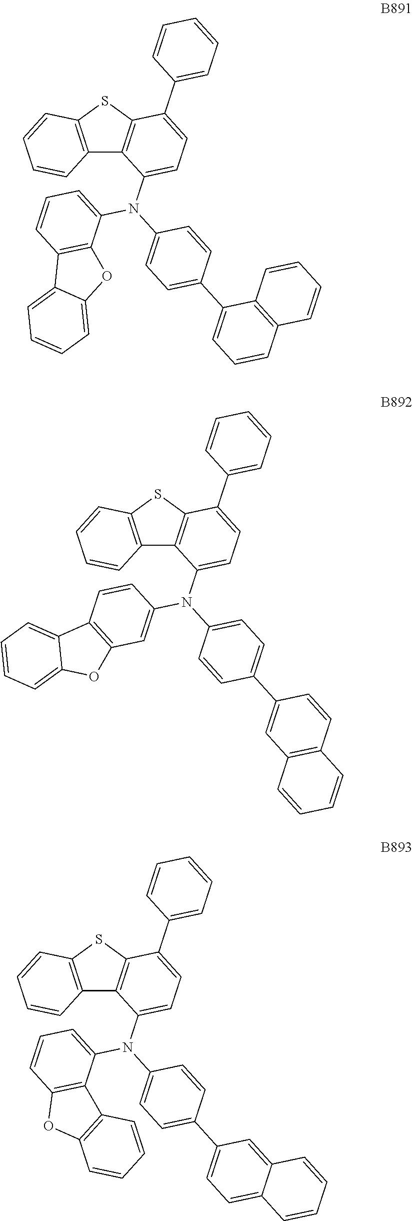 [SMILES: c1ccc(-c2ccc(N(c3ccc(-c4cccc5ccccc45)cc3)c3cccc4c3oc3ccccc34)c3c2sc2ccccc23)cc1 ; c1ccc(-c2ccc(N(c3ccc(-c4ccc5ccccc5c4)cc3)c3ccc4c(c3)oc3ccccc34)c3c2sc2ccccc23)cc1 ; c1ccc(-c2ccc(N(c3ccc(-c4ccc5ccccc5c4)cc3)c3cccc4oc5ccccc5c34)c3c2sc2ccccc23)cc1]

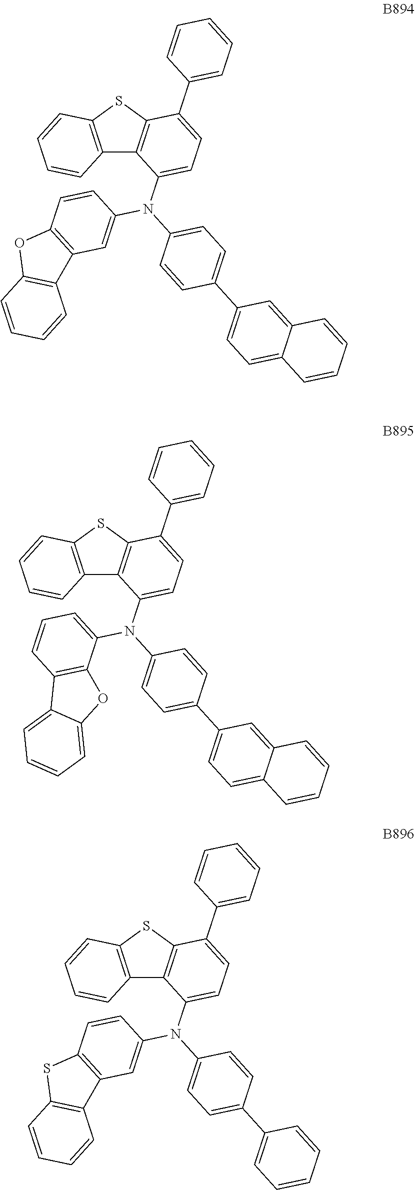 [SMILES: c1ccc(-c2ccc(N(c3ccc(-c4ccc5ccccc5c4)cc3)c3ccc4oc5ccccc5c4c3)c3c2sc2ccccc23)cc1 ; c1ccc(-c2ccc(N(c3ccc(-c4ccc5ccccc5c4)cc3)c3cccc4c3oc3ccccc34)c3c2sc2ccccc23)cc1 ; c1ccc(-c2ccc(N(c3ccc4sc5ccccc5c4c3)c3ccc(-c4ccccc4)c4sc5ccccc5c34)cc2)cc1]

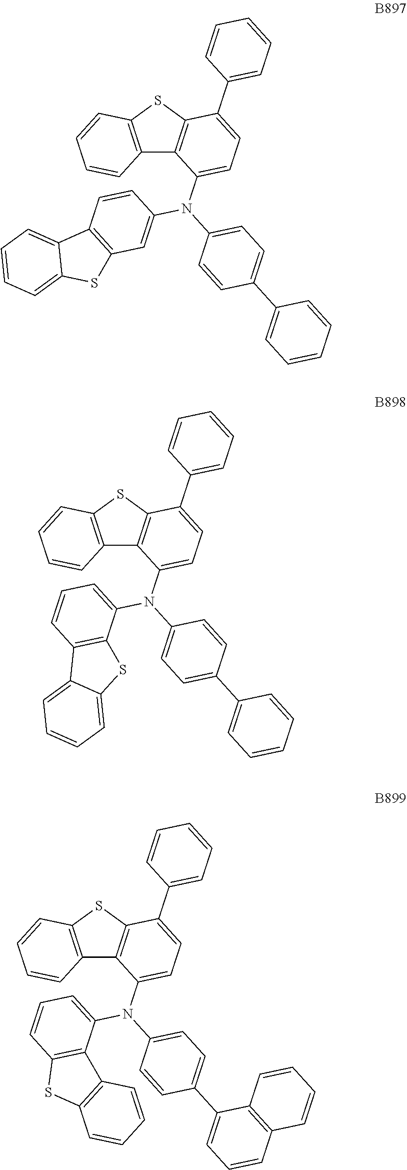 [SMILES: c1ccc(-c2ccc(N(c3ccc4c(c3)sc3ccccc34)c3ccc(-c4ccccc4)c4sc5ccccc5c34)cc2)cc1 ; c1ccc(-c2ccc(N(c3cccc4c3sc3ccccc34)c3ccc(-c4ccccc4)c4sc5ccccc5c34)cc2)cc1 ; c1ccc(-c2ccc(N(c3ccc(-c4cccc5ccccc45)cc3)c3cccc4sc5ccccc5c34)c3c2sc2ccccc23)cc1]

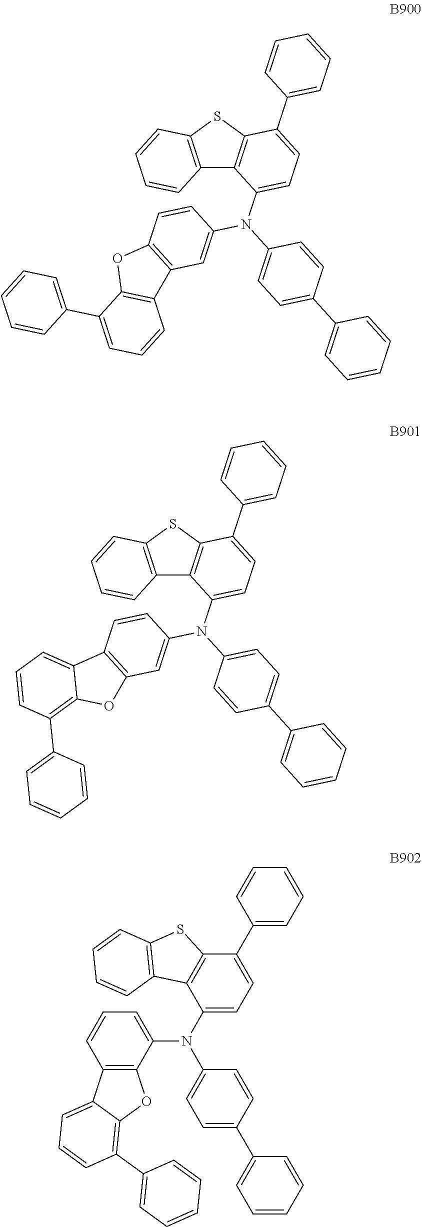 [SMILES: c1ccc(-c2ccc(N(c3ccc4oc5c(-c6ccccc6)cccc5c4c3)c3ccc(-c4ccccc4)c4sc5ccccc5c34)cc2)cc1 ; c1ccc(-c2ccc(N(c3ccc4c(c3)oc3c(-c5ccccc5)cccc34)c3ccc(-c4ccccc4)c4sc5ccccc5c34)cc2)cc1 ; c1ccc(-c2ccc(N(c3cccc4c3oc3c(-c5ccccc5)cccc34)c3ccc(-c4ccccc4)c4sc5ccccc5c34)cc2)cc1]

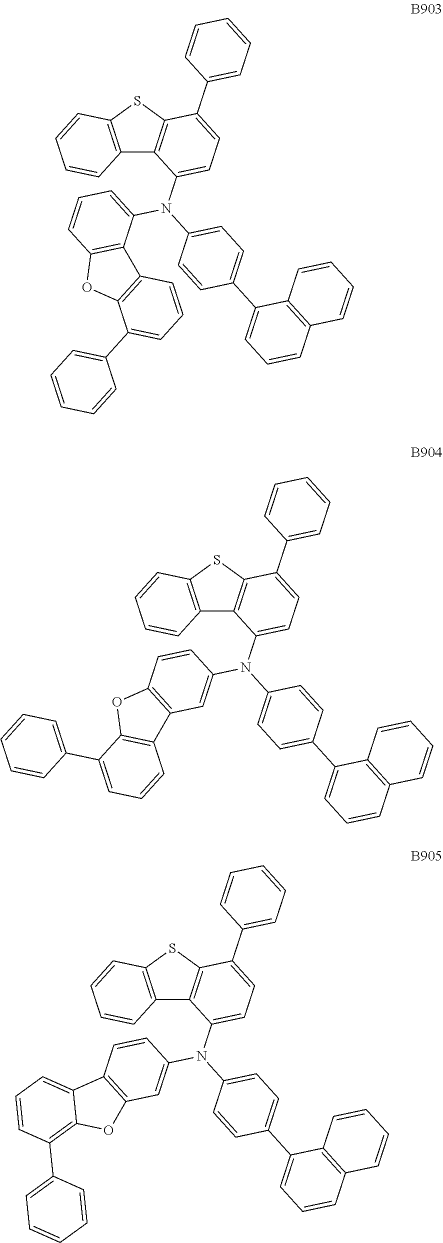 [SMILES: c1ccc(-c2cccc3c2oc2cccc(N(c4ccc(-c5cccc6ccccc56)cc4)c4ccc(-c5ccccc5)c5sc6ccccc6c45)c23)cc1 ; c1ccc(-c2cccc3c2oc2ccc(N(c4ccc(-c5cccc6ccccc56)cc4)c4ccc(-c5ccccc5)c5sc6ccccc6c45)cc23)cc1 ; c1ccc(-c2cccc3c2oc2cc(N(c4ccc(-c5cccc6ccccc56)cc4)c4ccc(-c5ccccc5)c5sc6ccccc6c45)ccc23)cc1]

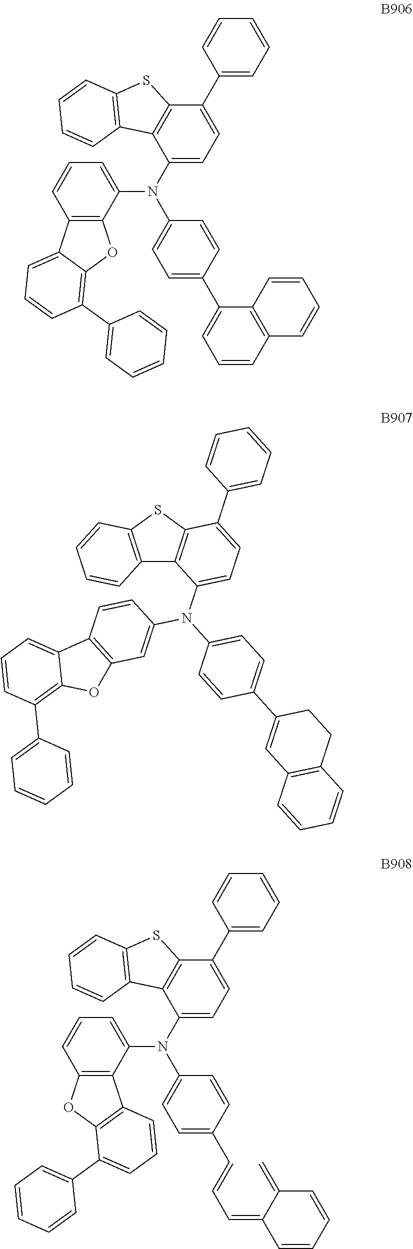 [SMILES: c1ccc(-c2cccc3c2oc2c(N(c4ccc(-c5cccc6ccccc56)cc4)c4ccc(-c5ccccc5)c5sc6ccccc6c45)cccc23)cc1 ; C1=C(c2ccc(N(c3ccc4c(c3)oc3c(-c5ccccc5)cccc34)c3ccc(-c4ccccc4)c4sc5ccccc5c34)cc2)CCc2ccccc21 ; C=c1cccc/c1=C/C=C/c1ccc(N(c2cccc3oc4c(-c5ccccc5)cccc4c23)c2ccc(-c3ccccc3)c3sc4ccccc4c23)cc1]

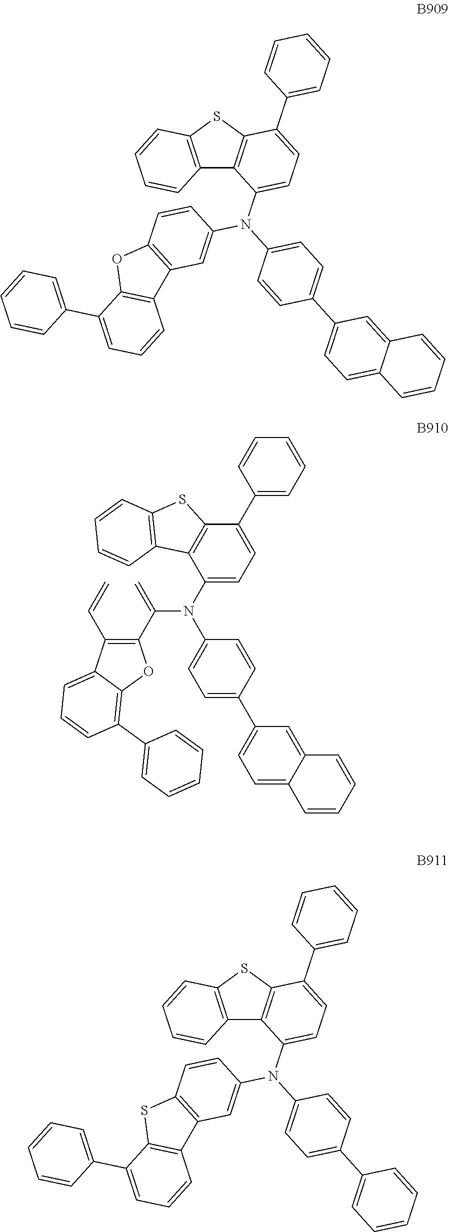 [SMILES: c1ccc(-c2cccc3c2oc2ccc(N(c4ccc(-c5ccc6ccccc6c5)cc4)c4ccc(-c5ccccc5)c5sc6ccccc6c45)cc23)cc1 ; C=Cc1c(C(=C)N(c2ccc(-c3ccc4ccccc4c3)cc2)c2ccc(-c3ccccc3)c3sc4ccccc4c23)oc2c(-c3ccccc3)cccc12 ; c1ccc(-c2ccc(N(c3ccc4sc5c(-c6ccccc6)cccc5c4c3)c3ccc(-c4ccccc4)c4sc5ccccc5c34)cc2)cc1]

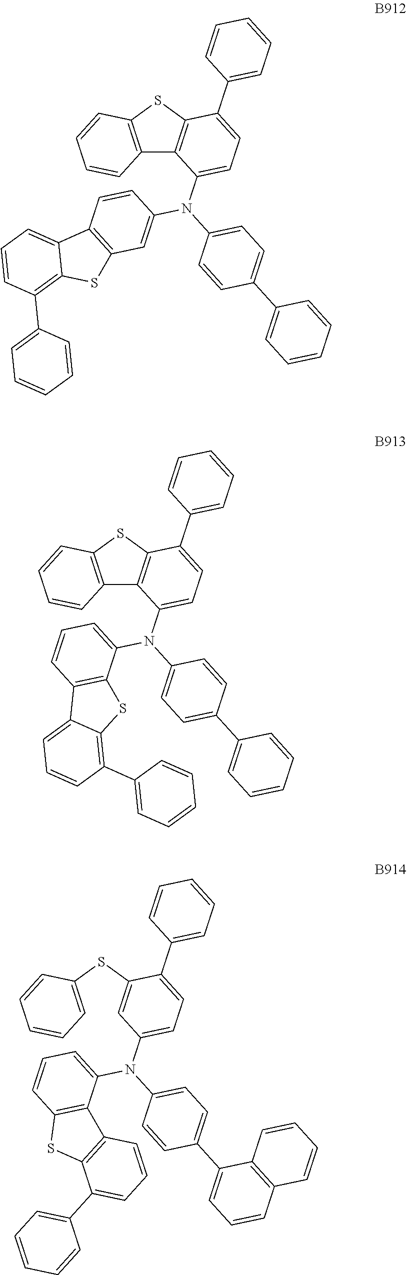 [SMILES: c1ccc(-c2ccc(N(c3ccc4c(c3)sc3c(-c5ccccc5)cccc34)c3ccc(-c4ccccc4)c4sc5ccccc5c34)cc2)cc1 ; c1ccc(-c2ccc(N(c3cccc4c3sc3c(-c5ccccc5)cccc34)c3ccc(-c4ccccc4)c4sc5ccccc5c34)cc2)cc1 ; c1ccc(Sc2cc(N(c3ccc(-c4cccc5ccccc45)cc3)c3cccc4sc5c(-c6ccccc6)cccc5c34)ccc2-c2ccccc2)cc1]

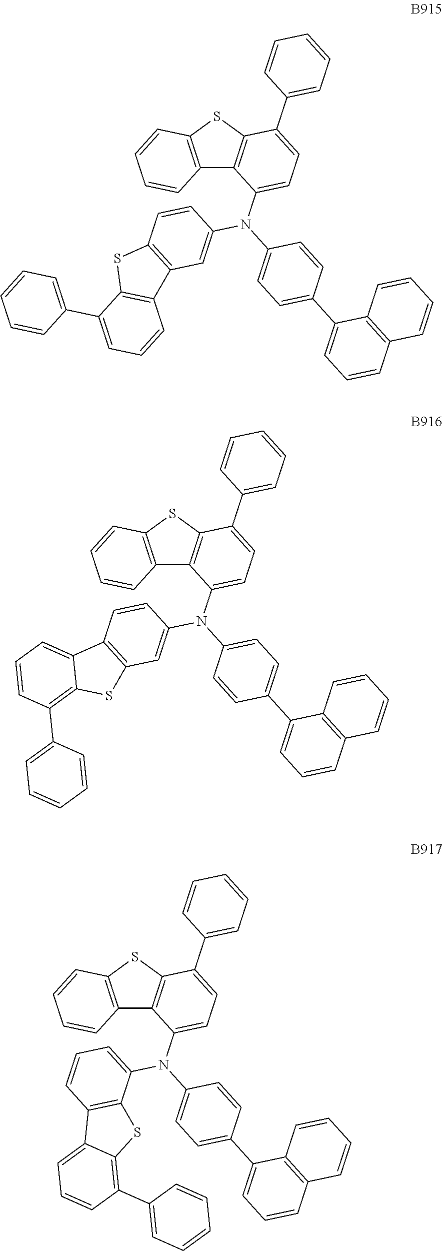 [SMILES: c1ccc(-c2cccc3c2sc2ccc(N(c4ccc(-c5cccc6ccccc56)cc4)c4ccc(-c5ccccc5)c5sc6ccccc6c45)cc23)cc1 ; c1ccc(-c2cccc3c2sc2cc(N(c4ccc(-c5cccc6ccccc56)cc4)c4ccc(-c5ccccc5)c5sc6ccccc6c45)ccc23)cc1 ; c1ccc(-c2cccc3c2sc2c(N(c4ccc(-c5cccc6ccccc56)cc4)c4ccc(-c5ccccc5)c5sc6ccccc6c45)cccc23)cc1]

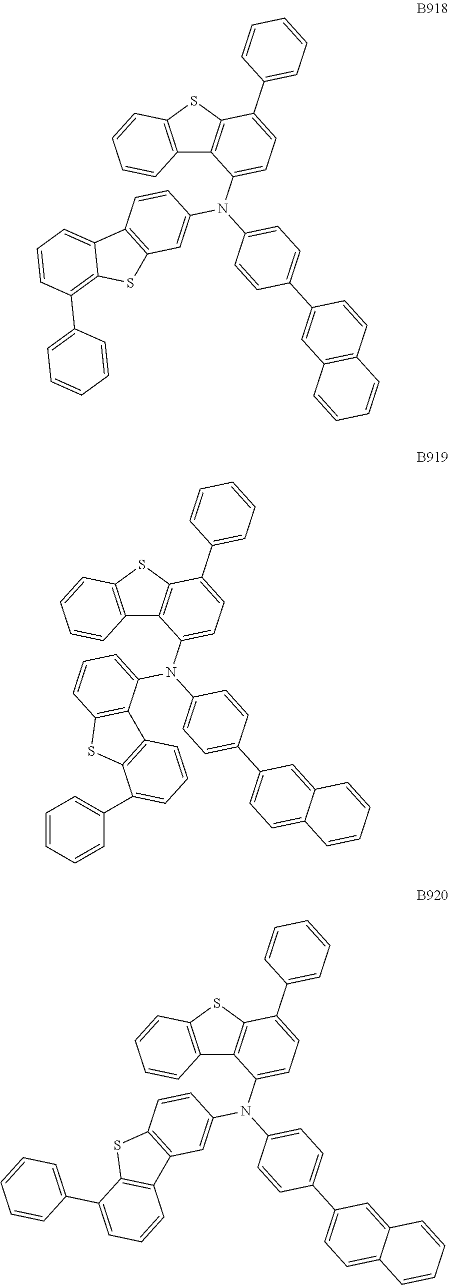 [SMILES: c1ccc(-c2cccc3c2sc2cc(N(c4ccc(-c5ccc6ccccc6c5)cc4)c4ccc(-c5ccccc5)c5sc6ccccc6c45)ccc23)cc1 ; c1ccc(-c2cccc3c2sc2cccc(N(c4ccc(-c5ccc6ccccc6c5)cc4)c4ccc(-c5ccccc5)c5sc6ccccc6c45)c23)cc1 ; c1ccc(-c2cccc3c2sc2ccc(N(c4ccc(-c5ccc6ccccc6c5)cc4)c4ccc(-c5ccccc5)c5sc6ccccc6c45)cc23)cc1]

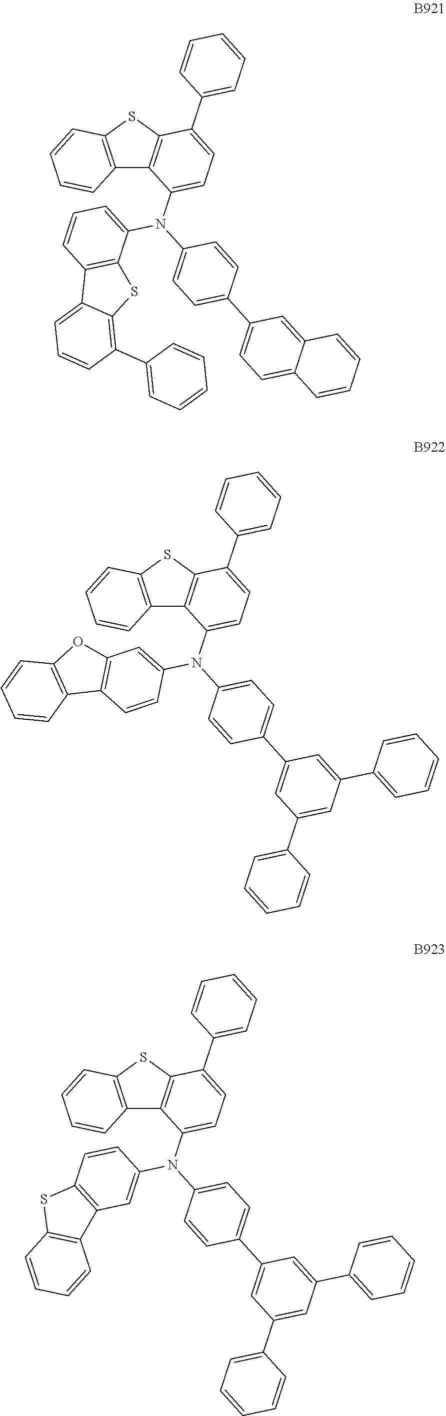 [SMILES: c1ccc(-c2cccc3c2sc2c(N(c4ccc(-c5ccc6ccccc6c5)cc4)c4ccc(-c5ccccc5)c5sc6ccccc6c45)cccc23)cc1 ; c1ccc(-c2cc(-c3ccccc3)cc(-c3ccc(N(c4ccc5c(c4)oc4ccccc45)c4ccc(-c5ccccc5)c5sc6ccccc6c45)cc3)c2)cc1 ; c1ccc(-c2cc(-c3ccccc3)cc(-c3ccc(N(c4ccc5sc6ccccc6c5c4)c4ccc(-c5ccccc5)c5sc6ccccc6c45)cc3)c2)cc1]

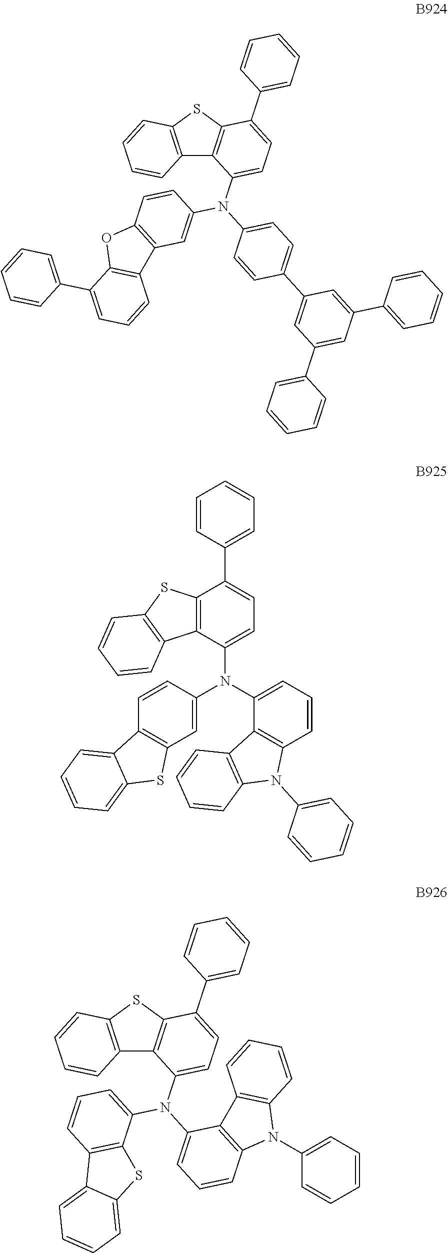 [SMILES: c1ccc(-c2cc(-c3ccccc3)cc(-c3ccc(N(c4ccc5oc6c(-c7ccccc7)cccc6c5c4)c4ccc(-c5ccccc5)c5sc6ccccc6c45)cc3)c2)cc1 ; c1ccc(-c2ccc(N(c3ccc4c(c3)sc3ccccc34)c3cccc4c3c3ccccc3n4-c3ccccc3)c3c2sc2ccccc23)cc1 ; c1ccc(-c2ccc(N(c3cccc4c3sc3ccccc34)c3cccc4c3c3ccccc3n4-c3ccccc3)c3c2sc2ccccc23)cc1]

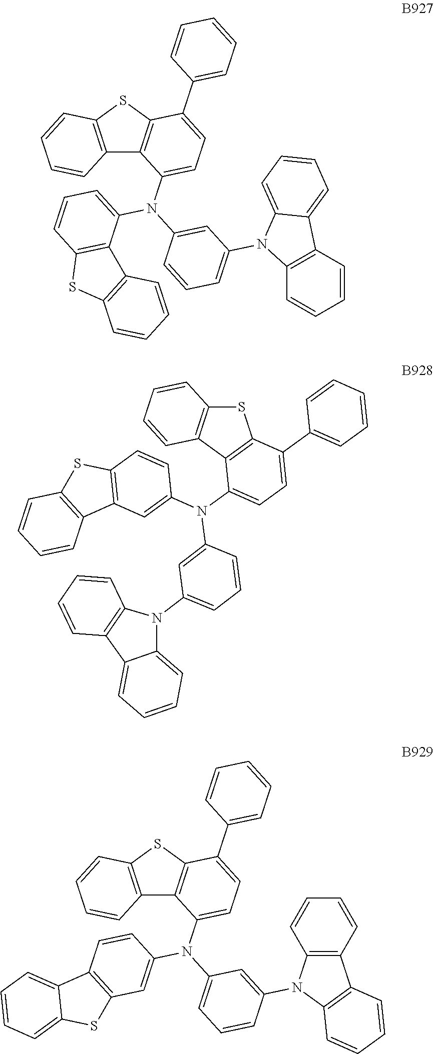 [SMILES: c1ccc(-c2ccc(N(c3cccc(-n4c5ccccc5c5ccccc54)c3)c3cccc4sc5ccccc5c34)c3c2sc2ccccc23)cc1 ; c1ccc(-c2ccc(N(c3cccc(-n4c5ccccc5c5ccccc54)c3)c3ccc4sc5ccccc5c4c3)c3c2sc2ccccc23)cc1 ; c1ccc(-c2ccc(N(c3cccc(-n4c5ccccc5c5ccccc54)c3)c3ccc4c(c3)sc3ccccc34)c3c2sc2ccccc23)cc1]

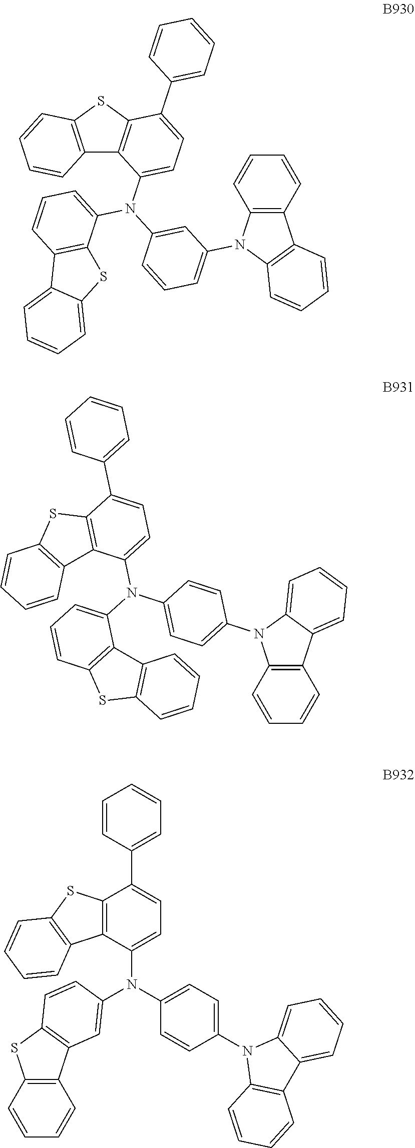 [SMILES: c1ccc(-c2ccc(N(c3cccc(-n4c5ccccc5c5ccccc54)c3)c3cccc4c3sc3ccccc34)c3c2sc2ccccc23)cc1 ; c1ccc(-c2ccc(N(c3ccc(-n4c5ccccc5c5ccccc54)cc3)c3cccc4sc5ccccc5c34)c3c2sc2ccccc23)cc1 ; c1ccc(-c2ccc(N(c3ccc(-n4c5ccccc5c5ccccc54)cc3)c3ccc4sc5ccccc5c4c3)c3c2sc2ccccc23)cc1]

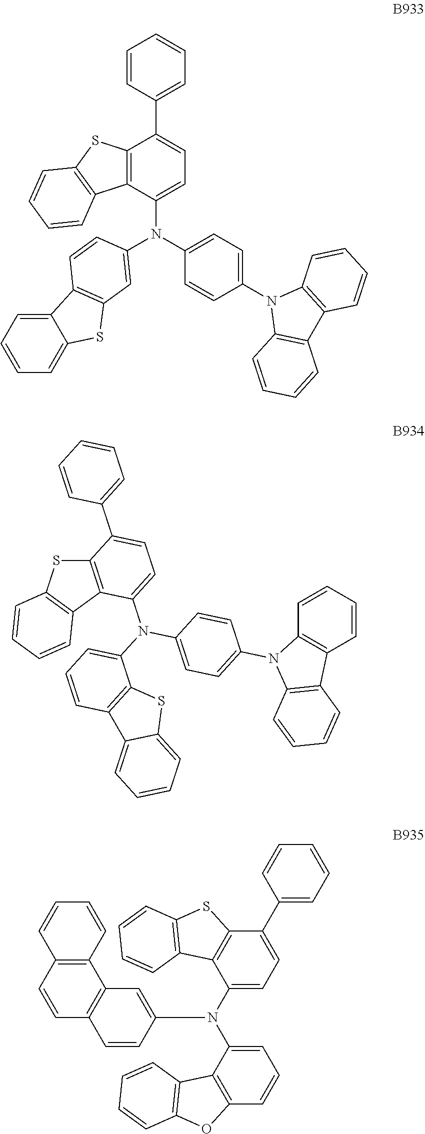 [SMILES: c1ccc(-c2ccc(N(c3ccc(-n4c5ccccc5c5ccccc54)cc3)c3ccc4c(c3)sc3ccccc34)c3c2sc2ccccc23)cc1 ; c1ccc(-c2ccc(N(c3ccc(-n4c5ccccc5c5ccccc54)cc3)c3cccc4c3sc3ccccc34)c3c2sc2ccccc23)cc1 ; c1ccc(-c2ccc(N(c3ccc4ccc5ccccc5c4c3)c3cccc4oc5ccccc5c34)c3c2sc2ccccc23)cc1]

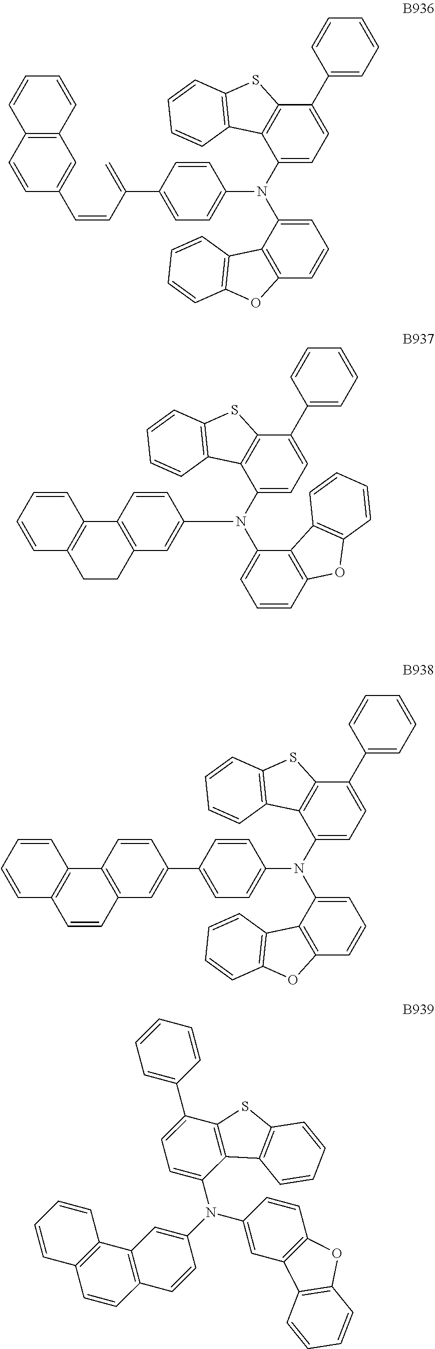 [SMILES: C=C(/C=C\c1ccc2ccccc2c1)c1ccc(N(c2cccc3oc4ccccc4c23)c2ccc(-c3ccccc3)c3sc4ccccc4c23)cc1 ; c1ccc(-c2ccc(N(c3ccc4c(c3)CCc3ccccc3-4)c3cccc4oc5ccccc5c34)c3c2sc2ccccc23)cc1 ; c1ccc(-c2ccc(N(c3ccc(-c4ccc5c(ccc6ccccc65)c4)cc3)c3cccc4oc5ccccc5c34)c3c2sc2ccccc23)cc1 ; c1ccc(-c2ccc(N(c3ccc4ccc5ccccc5c4c3)c3ccc4oc5ccccc5c4c3)c3c2sc2ccccc23)cc1]

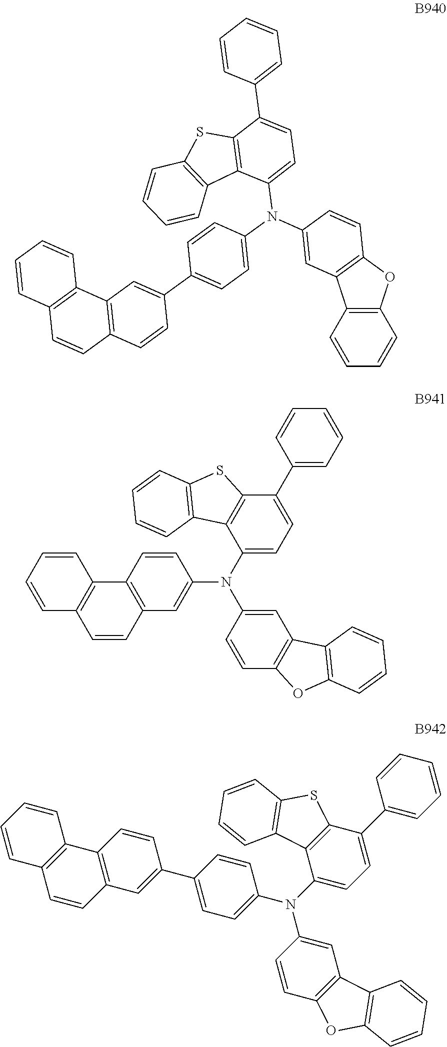 [SMILES: c1ccc(-c2ccc(N(c3ccc(-c4ccc5ccc6ccccc6c5c4)cc3)c3ccc4oc5ccccc5c4c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1 ; c1ccc(-c2ccc(N(c3ccc(-c4ccc5c(ccc6ccccc65)c4)cc3)c3ccc4oc5ccccc5c4c3)c3c2sc2ccccc23)cc1]

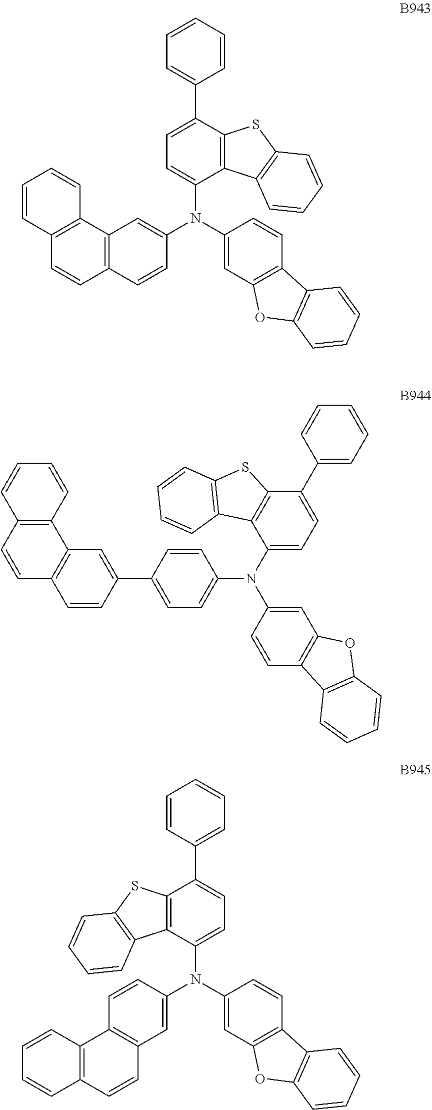 [SMILES: c1ccc(-c2ccc(N(c3ccc4c(c3)oc3ccccc34)c3ccc4ccc5ccccc5c4c3)c3c2sc2ccccc23)cc1 ; c1ccc(-c2ccc(N(c3ccc(-c4ccc5ccc6ccccc6c5c4)cc3)c3ccc4c(c3)oc3ccccc34)c3c2sc2ccccc23)cc1 ; c1ccc(-c2ccc(N(c3ccc4c(ccc5ccccc54)c3)c3ccc4c(c3)oc3ccccc34)c3c2sc2ccccc23)cc1]

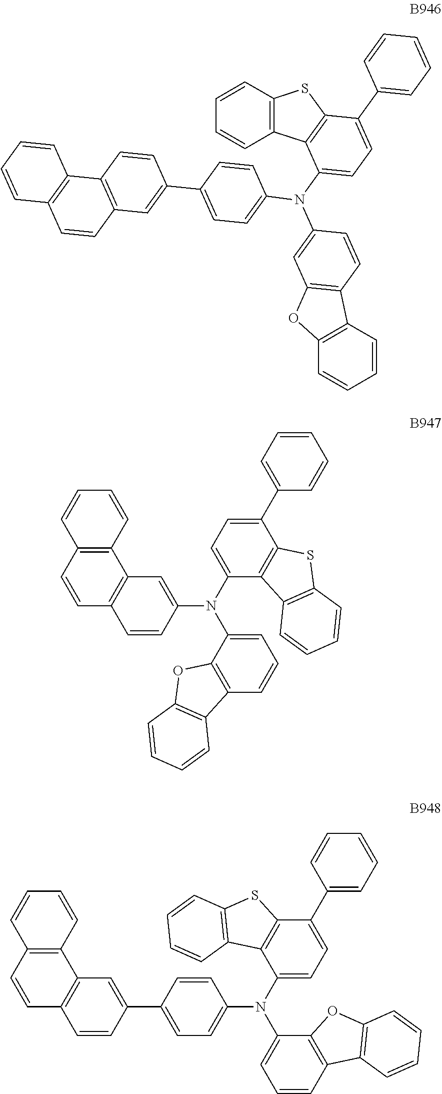 [SMILES: c1ccc(-c2ccc(N(c3ccc(-c4ccc5c(ccc6ccccc65)c4)cc3)c3ccc4c(c3)oc3ccccc34)c3c2sc2ccccc23)cc1 ; c1ccc(-c2ccc(N(c3ccc4ccc5ccccc5c4c3)c3cccc4c3oc3ccccc34)c3c2sc2ccccc23)cc1 ; c1ccc(-c2ccc(N(c3ccc(-c4ccc5ccc6ccccc6c5c4)cc3)c3cccc4c3oc3ccccc34)c3c2sc2ccccc23)cc1]

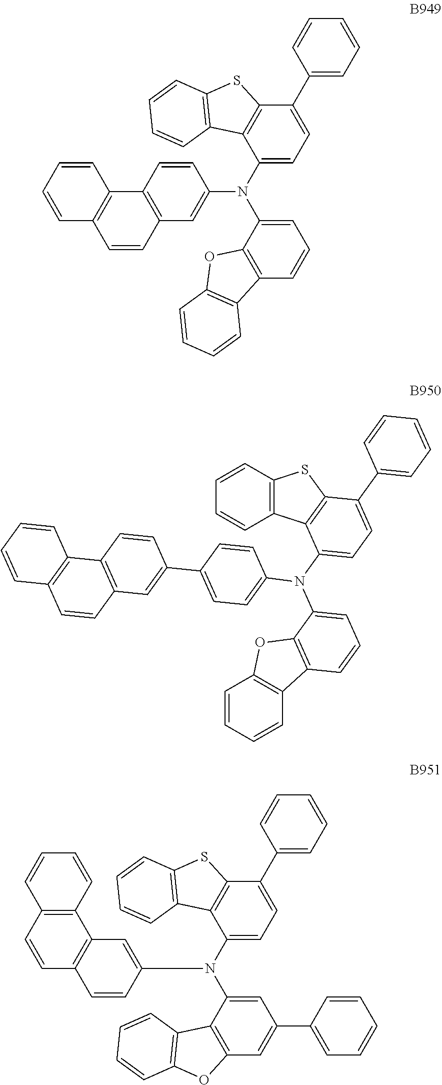 [SMILES: c1ccc(-c2ccc(N(c3ccc4c(ccc5ccccc54)c3)c3cccc4c3oc3ccccc34)c3c2sc2ccccc23)cc1 ; c1ccc(-c2ccc(N(c3ccc(-c4ccc5c(ccc6ccccc65)c4)cc3)c3cccc4c3oc3ccccc34)c3c2sc2ccccc23)cc1 ; c1ccc(-c2cc(N(c3ccc4ccc5ccccc5c4c3)c3ccc(-c4ccccc4)c4sc5ccccc5c34)c3c(c2)oc2ccccc23)cc1]

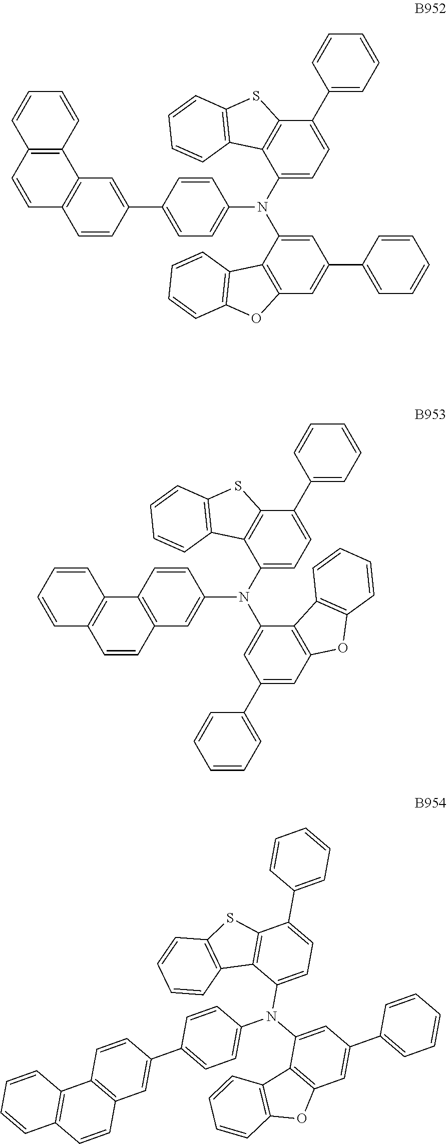 [SMILES: c1ccc(-c2cc(N(c3ccc(-c4ccc5ccc6ccccc6c5c4)cc3)c3ccc(-c4ccccc4)c4sc5ccccc5c34)c3c(c2)oc2ccccc23)cc1 ; c1ccc(-c2cc(N(c3ccc4c(ccc5ccccc54)c3)c3ccc(-c4ccccc4)c4sc5ccccc5c34)c3c(c2)oc2ccccc23)cc1 ; c1ccc(-c2cc(N(c3ccc(-c4ccc5c(ccc6ccccc65)c4)cc3)c3ccc(-c4ccccc4)c4sc5ccccc5c34)c3c(c2)oc2ccccc23)cc1]

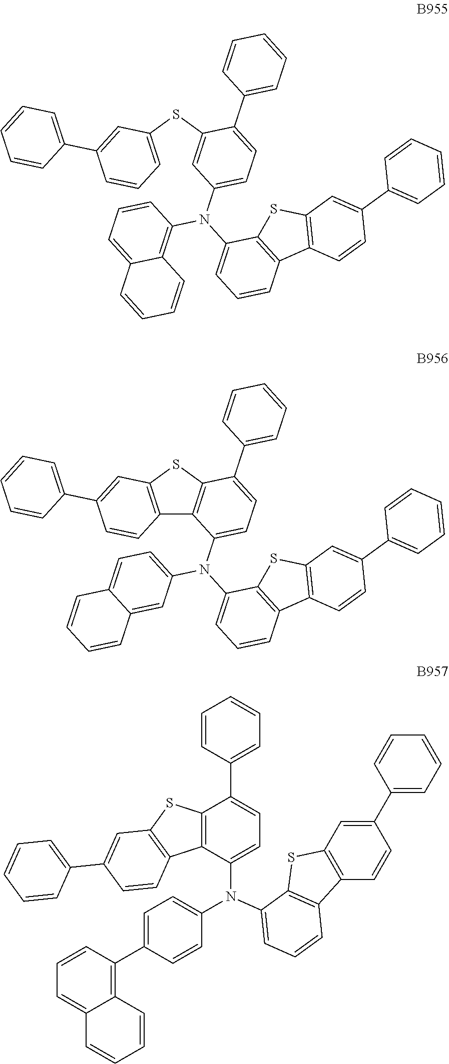 [SMILES: c1ccc(-c2cccc(Sc3cc(N(c4cccc5ccccc45)c4cccc5c4sc4cc(-c6ccccc6)ccc45)ccc3-c3ccccc3)c2)cc1 ; c1ccc(-c2ccc3c(c2)sc2c(N(c4ccc5ccccc5c4)c4ccc(-c5ccccc5)c5sc6cc(-c7ccccc7)ccc6c45)cccc23)cc1 ; c1ccc(-c2ccc3c(c2)sc2c(N(c4ccc(-c5cccc6ccccc56)cc4)c4ccc(-c5ccccc5)c5sc6cc(-c7ccccc7)ccc6c45)cccc23)cc1]

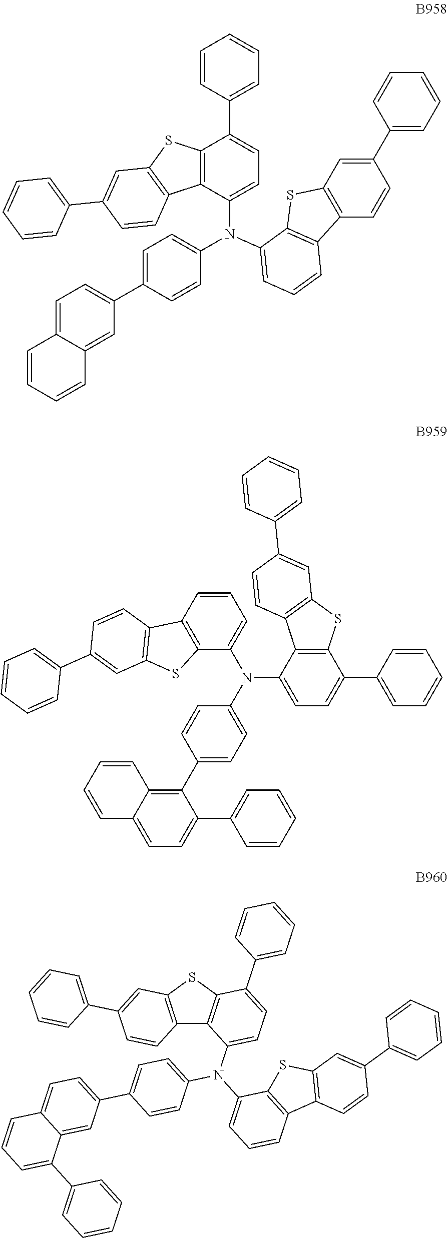 [SMILES: c1ccc(-c2ccc3c(c2)sc2c(N(c4ccc(-c5ccc6ccccc6c5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5c(-c6ccccc6)ccc6ccccc56)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6cccc(-c7ccccc7)c6c5)cc4)c4ccc(-c5ccccc5)c5sc6cc(-c7ccccc7)ccc6c45)cccc23)cc1]

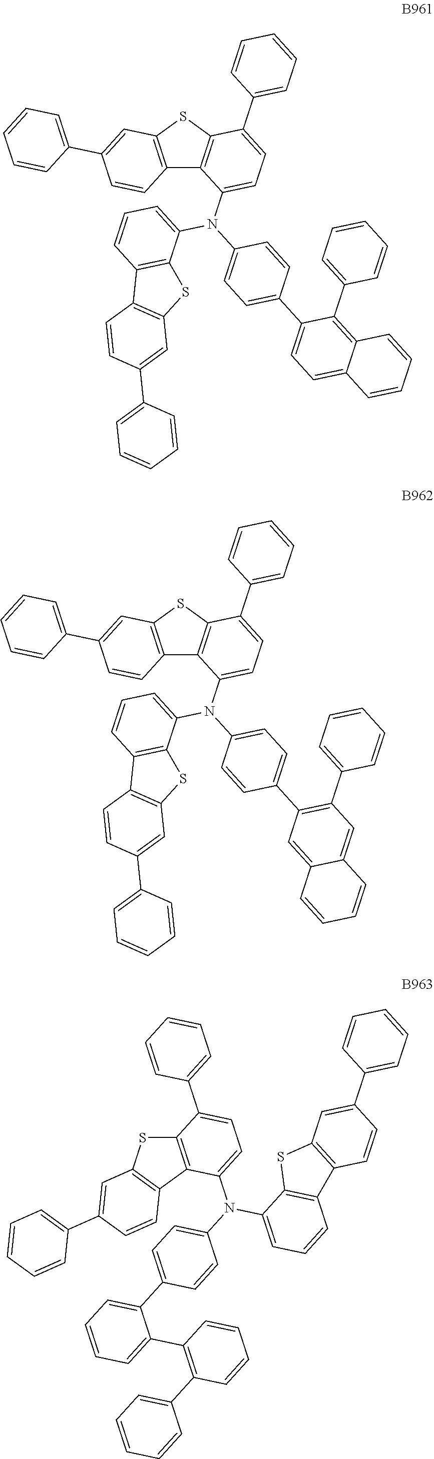 [SMILES: c1ccc(-c2ccc3c(c2)sc2c(N(c4ccc(-c5ccc6ccccc6c5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5cc6ccccc6cc5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccccc5-c5ccccc5-c5ccccc5)cc4)c4ccc(-c5ccccc5)c5sc6cc(-c7ccccc7)ccc6c45)cccc23)cc1]

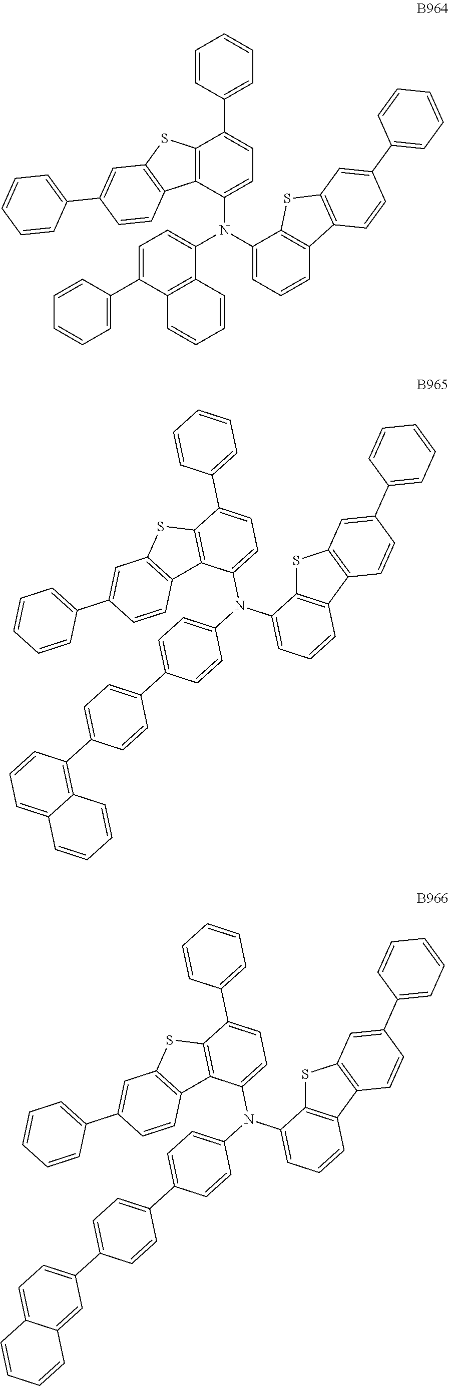 [SMILES: c1ccc(-c2ccc3c(c2)sc2c(N(c4ccc(-c5ccccc5)c5ccccc45)c4ccc(-c5ccccc5)c5sc6cc(-c7ccccc7)ccc6c45)cccc23)cc1 ; c1ccc(-c2ccc3c(c2)sc2c(N(c4ccc(-c5ccc(-c6cccc7ccccc67)cc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccc(-c6ccc7ccccc7c6)cc5)cc4)c4ccc(-c5ccccc5)c5sc6cc(-c7ccccc7)ccc6c45)cccc23)cc1]

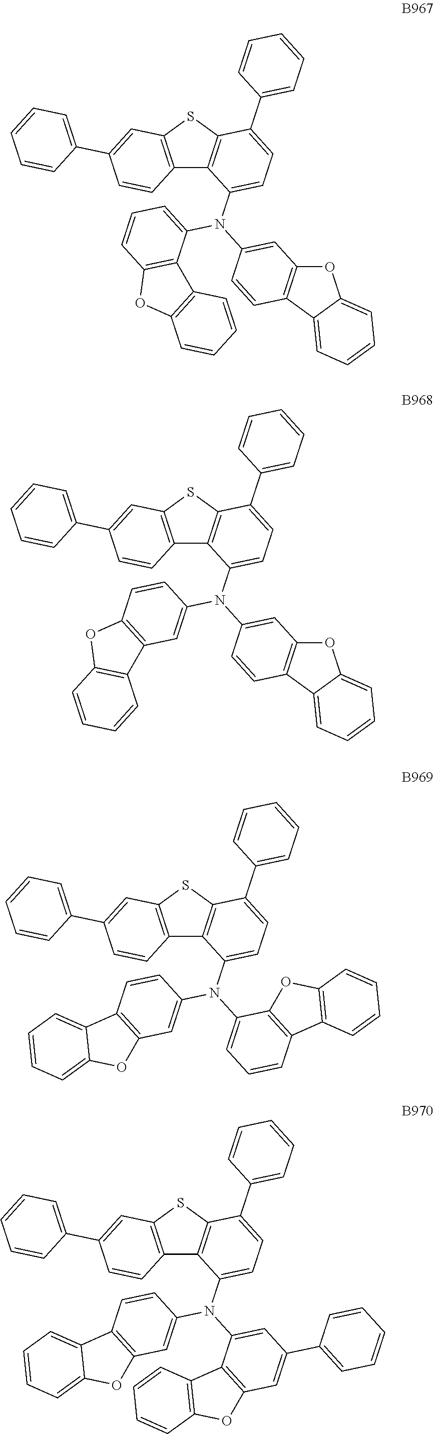 [SMILES: c1ccc(-c2ccc3c(c2)sc2c(-c4ccccc4)ccc(N(c4ccc5c(c4)oc4ccccc45)c4cccc5oc6ccccc6c45)c23)cc1 ; c1ccc(-c2ccc3c(c2)sc2c(-c4ccccc4)ccc(N(c4ccc5c(c4)oc4ccccc45)c4ccc5oc6ccccc6c5c4)c23)cc1 ; c1ccc(-c2ccc3c(c2)sc2c(-c4ccccc4)ccc(N(c4ccc5c(c4)oc4ccccc45)c4cccc5c4oc4ccccc45)c23)cc1 ; c1ccc(-c2cc(N(c3ccc4c(c3)oc3ccccc34)c3ccc(-c4ccccc4)c4sc5cc(-c6ccccc6)ccc5c34)c3c(c2)oc2ccccc23)cc1]

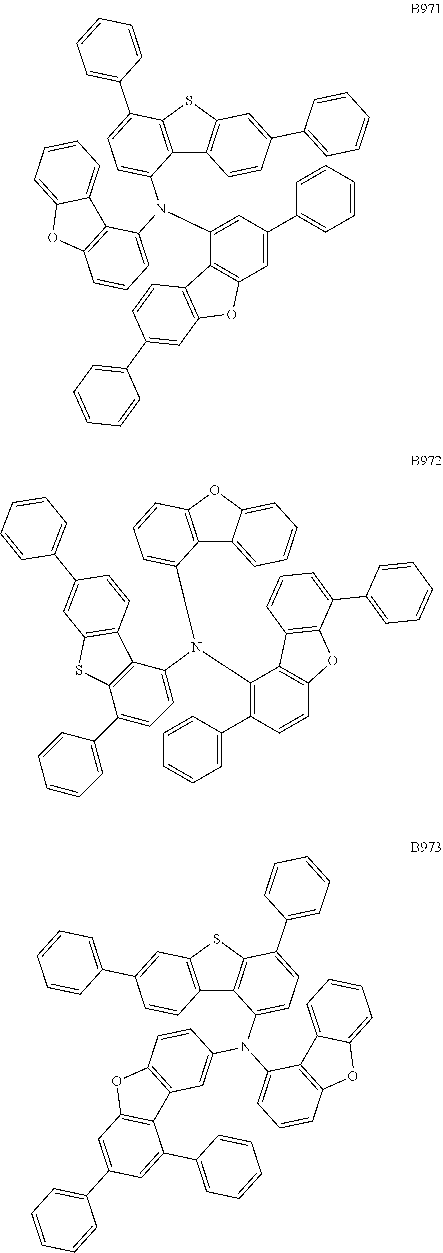 [SMILES: c1ccc(-c2ccc3c(c2)oc2cc(-c4ccccc4)cc(N(c4cccc5oc6ccccc6c45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5oc6ccccc6c45)c4c(-c5ccccc5)ccc5oc6c(-c7ccccc7)cccc6c45)c23)cc1 ; c1ccc(-c2cc(-c3ccccc3)c3c(c2)oc2ccc(N(c4cccc5oc6ccccc6c45)c4ccc(-c5ccccc5)c5sc6cc(-c7ccccc7)ccc6c45)cc23)cc1]

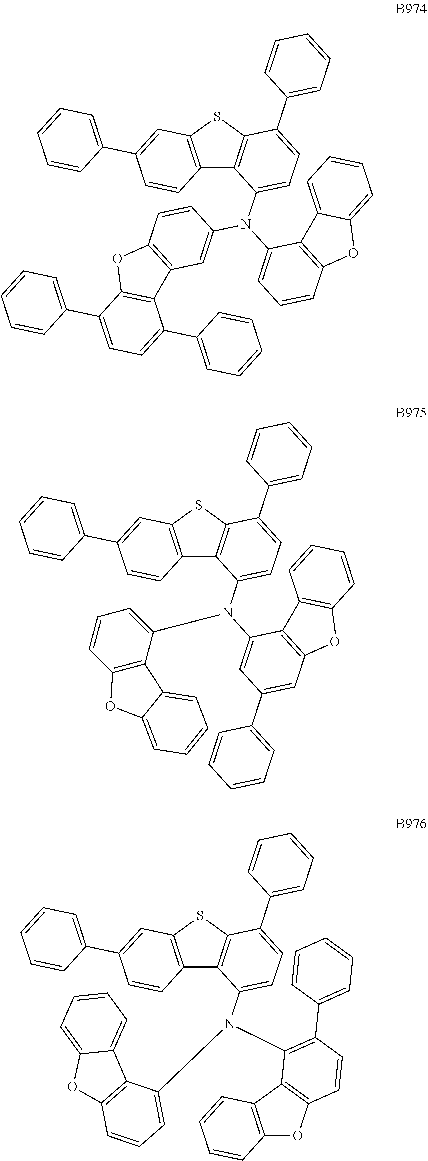 [SMILES: c1ccc(-c2ccc3c(c2)sc2c(-c4ccccc4)ccc(N(c4ccc5oc6c(-c7ccccc7)ccc(-c7ccccc7)c6c5c4)c4cccc5oc6ccccc6c45)c23)cc1 ; c1ccc(-c2cc(N(c3cccc4oc5ccccc5c34)c3ccc(-c4ccccc4)c4sc5cc(-c6ccccc6)ccc5c34)c3c(c2)oc2ccccc23)cc1 ; c1ccc(-c2ccc3c(c2)sc2c(-c4ccccc4)ccc(N(c4cccc5oc6ccccc6c45)c4c(-c5ccccc5)ccc5oc6ccccc6c45)c23)cc1]

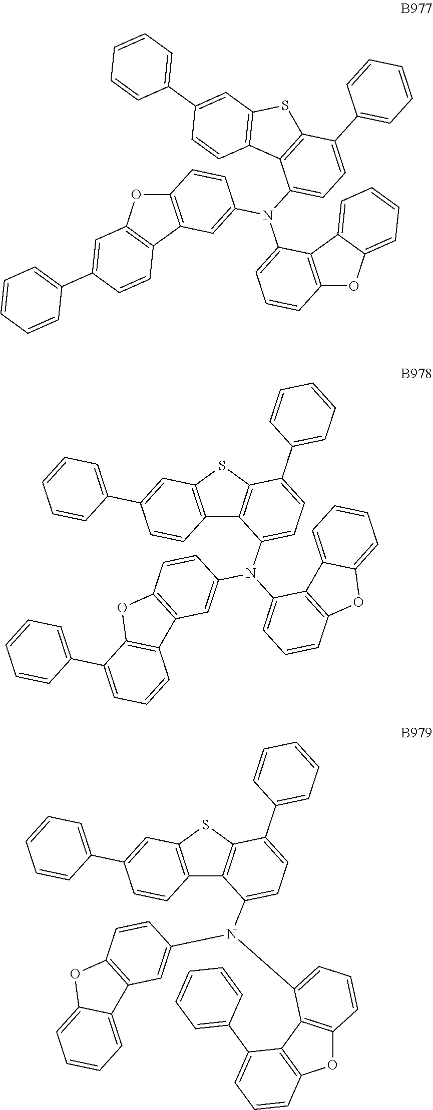 [SMILES: c1ccc(-c2ccc3c(c2)oc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4cccc5oc6cccc(-c7ccccc7)c6c45)c23)cc1]

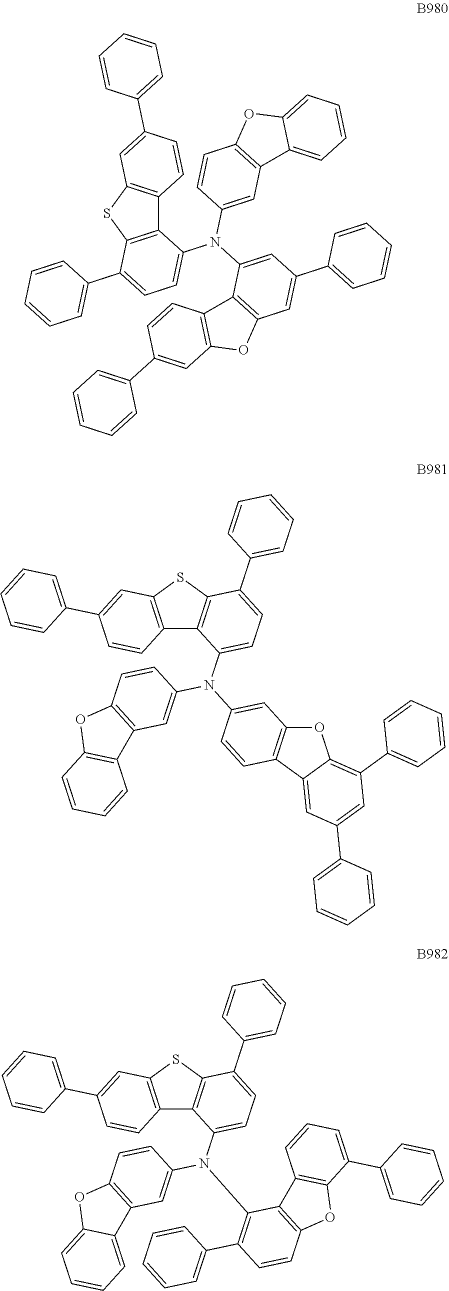 [SMILES: c1ccc(-c2ccc3c(c2)oc2cc(-c4ccccc4)cc(N(c4ccc5oc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)oc4c(-c6ccccc6)cc(-c6ccccc6)cc45)c4ccc5oc6ccccc6c5c4)c23)cc1 ; c1ccc(-c2ccc3c(c2)sc2c(-c4ccccc4)ccc(N(c4ccc5oc6ccccc6c5c4)c4c(-c5ccccc5)ccc5oc6c(-c7ccccc7)cccc6c45)c23)cc1]

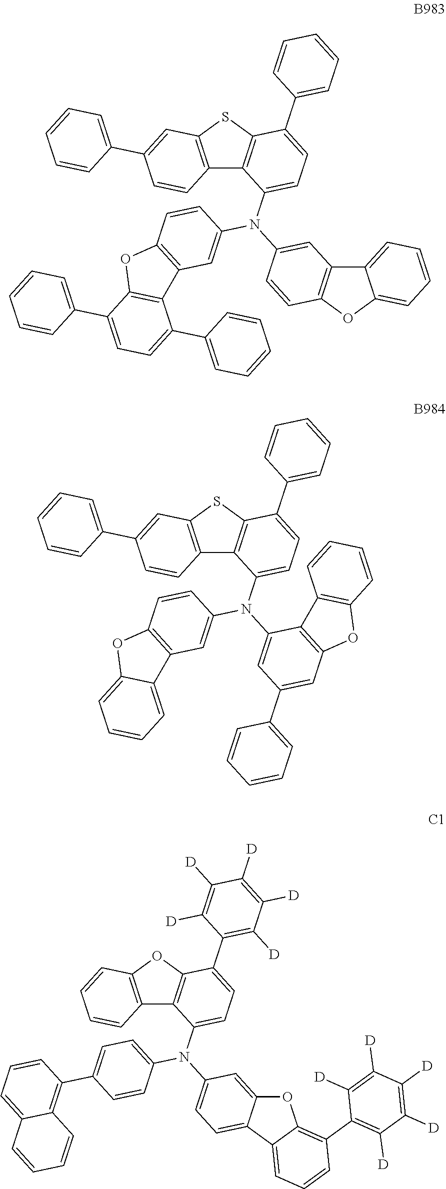 [SMILES: c1ccc(-c2ccc3c(c2)sc2c(-c4ccccc4)ccc(N(c4ccc5oc6ccccc6c5c4)c4ccc5oc6c(-c7ccccc7)ccc(-c7ccccc7)c6c5c4)c23)cc1 ; c1ccc(-c2cc(N(c3ccc4oc5ccccc5c4c3)c3ccc(-c4ccccc4)c4sc5cc(-c6ccccc6)ccc5c34)c3c(c2)oc2ccccc23)cc1 ; [2H]c1c([2H])c([2H])c(-c2cccc3c2oc2cc(N(c4ccc(-c5cccc6ccccc56)cc4)c4ccc(-c5c([2H])c([2H])c([2H])c([2H])c5[2H])c5oc6ccccc6c45)ccc23)c([2H])c1[2H]]

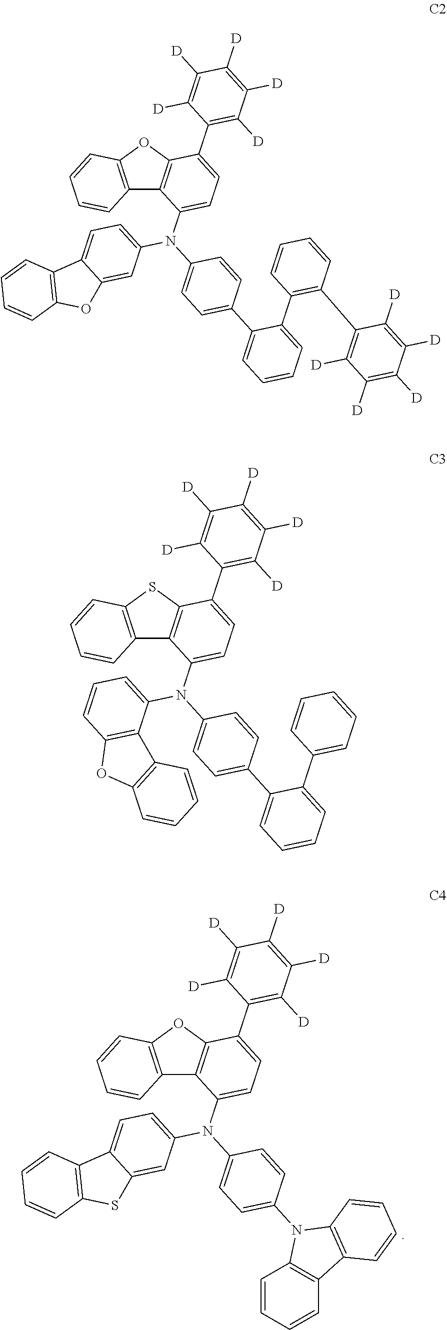 [SMILES: [2H]c1c([2H])c([2H])c(-c2ccccc2-c2ccccc2-c2ccc(N(c3ccc4c(c3)oc3ccccc34)c3ccc(-c4c([2H])c([2H])c([2H])c([2H])c4[2H])c4oc5ccccc5c34)cc2)c([2H])c1[2H] ; [2H]c1c([2H])c([2H])c(-c2ccc(N(c3ccc(-c4ccccc4-c4ccccc4)cc3)c3cccc4oc5ccccc5c34)c3c2sc2ccccc23)c([2H])c1[2H] ; [2H]c1c([2H])c([2H])c(-c2ccc(N(c3ccc(-n4c5ccccc5c5ccccc54)cc3)c3ccc4c(c3)sc3ccccc34)c3c2oc2ccccc23)c([2H])c1[2H]]

The amine compound according to an embodiment including a first substituent, a second substituent and a third substituent, that are connected to or with a core nitrogen atom, may achieve or accomplish high efficiency (e.g., high luminance emission efficiency), low voltage, and/or long lifetime when utilized in a light emitting element.

The amine compound of an embodiment includes an amine group and has a structure in which the first substituent, the second substituent, and the third substituent are connected to or combined with the amine group (i.e., the core nitrogen atom) of the amine compound. In an embodiment, each of the first and second substituents includes, or essentially includes, a dibenzoheterole moiety. The first substituent is directly connected, or bonded, to the core nitrogen atom at a carbon position 1, and an aryl group may be substituted, or essentially substituted, in any position among carbon atoms at positions 2 to 4 of the first substituent. The second substituent is directly connected to or with the core nitrogen atom. The third substituent is connected to or with the core nitrogen atom via an arylene linker, or directly connected to or with the core nitrogen atom without a separate (e.g., arylene) linker.

The amine compound of an embodiment, having the structure described herein includes the first substituent that is, or having, a dibenzoheterole structure in which an aryl group is connected to (i.e., substituted in) any one carbon among carbon atoms at positions 2 to 4, and the first substituent is connected to or with the core nitrogen atom at carbon position 1. Accordingly, conjugation in a molecule of the amine compound of an embodiment increases to achieve, or show, excellent or suitable electrical stability and high charge transport capacity, and thus, when utilized in, or applied to, a light emitting element, the luminance emission efficiency and lifetime of the element may be enhanced or improved. In some embodiments, in the first substituent, an aryl group is connected to (i.e., substituted in) any one carbon among carbon atoms at positions 2 to 4, and, regarding the amine compound of an embodiment, intermolecular stacking may be enhanced or improved (e.g., may occur easily), intermolecular distance may be reduced or improved, and/or hole transport capacity may be enhanced or improved. The amine compound of an embodiment includes the second substituent having a dibenzoheterole structure. Accordingly, the hole transport capacity of the amine compound may be additionally enhanced or increased, and the stability of a radical cation state of the amine compound may be enhanced or improved. Accordingly, when the amine compound according to an embodiment of the present disclosure is utilized in, or applied to, the hole transport region HTR of the light emitting element ED, a light emitting element with high efficiency (e.g., luminance emission efficiency), low driving voltage, high luminance and long lifetime may be enabled or accomplished.

In the light emitting element ED of an embodiment, the hole transport region HTR may further include a compound represented by Formula H-1.

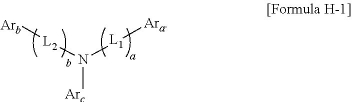

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. In Formula H, -a and b may each independently be an integer of 0 to 10. In some embodiments, when a or b is an integer of 2 or more, multiple L₁ and L₂ may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ara and Arb may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Arc may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one among Ara to Arc includes an amine group as a substituent. In some embodiments, the compound represented by Formula H-1 may be a carbazole-based compound in which at least one among Ara and Arb includes a substituted or unsubstituted carbazole group, or a fluorene-based compound in which at least one among Ara and Arb includes a substituted or unsubstituted fluorene group.

The compound represented by Formula H-1 may be represented by any one selected from among the compounds in Compound Group H. However, the compounds listed in Compound Group H are only illustrations, and the compound represented by Formula H-1 is not limited to the compounds represented in Compound Group H.

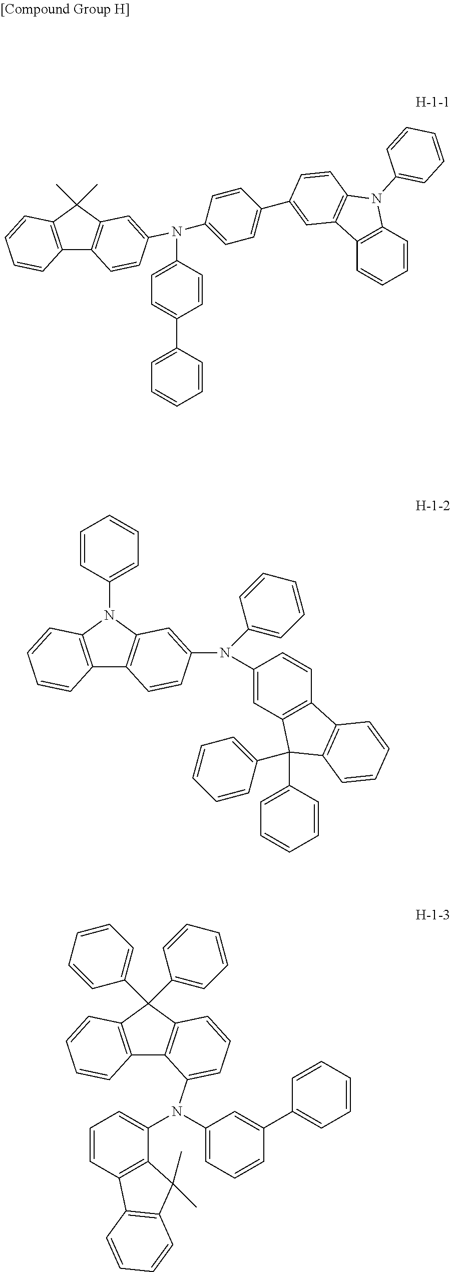 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

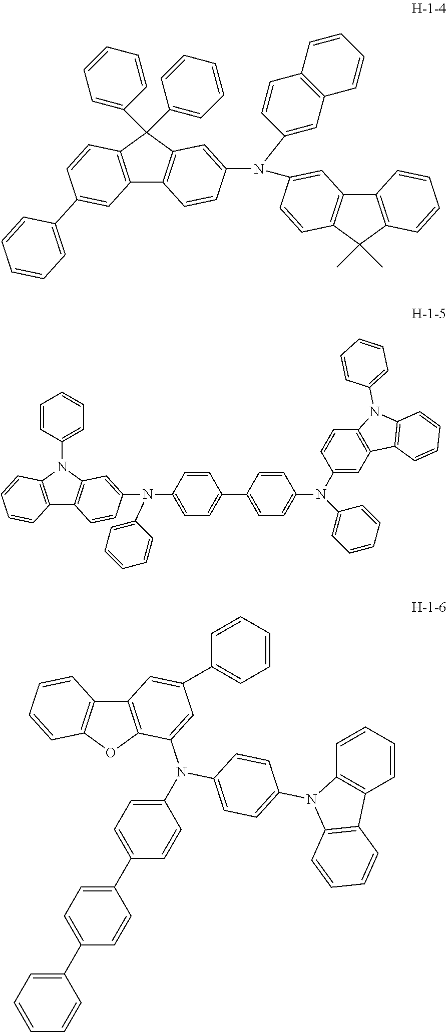 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1]

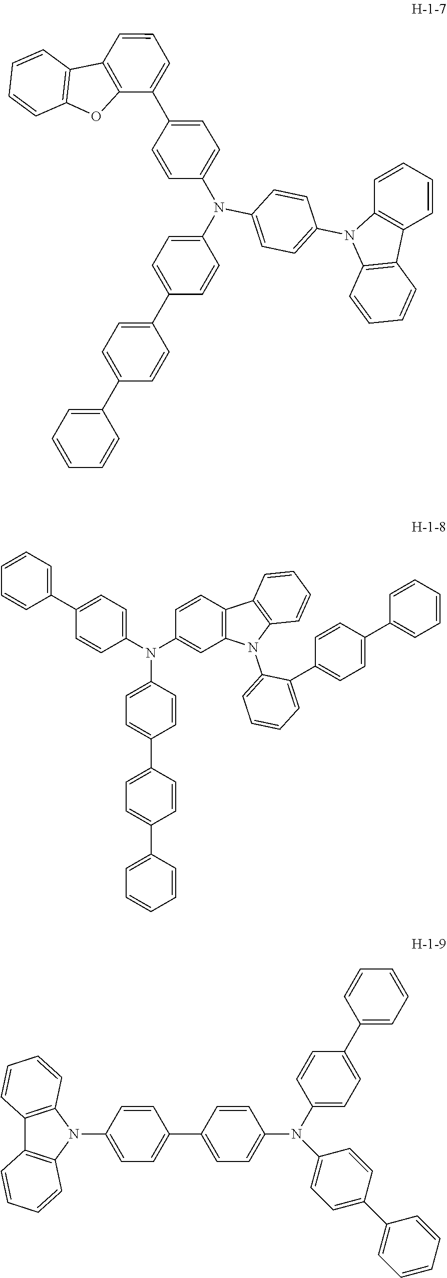 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

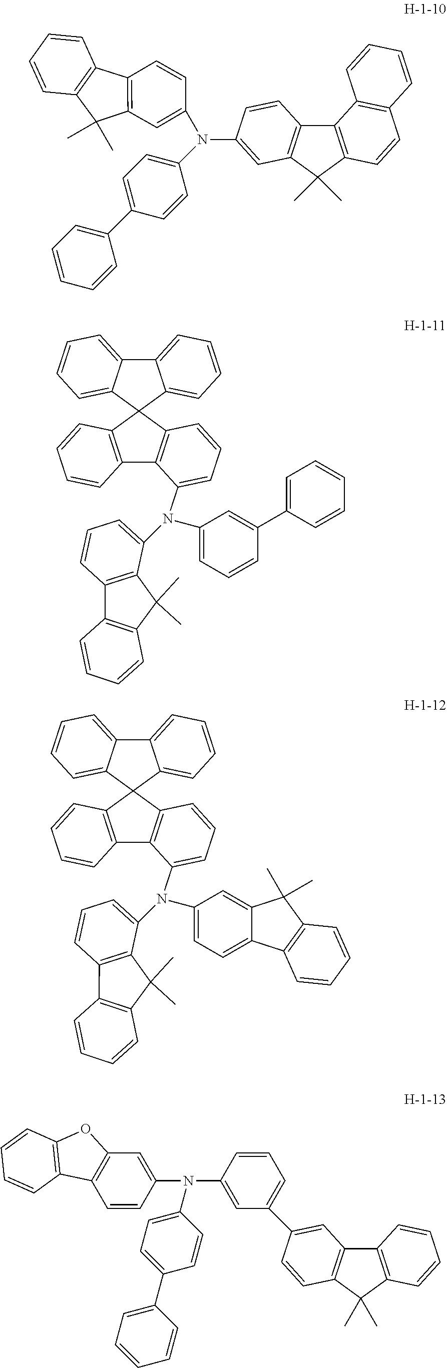

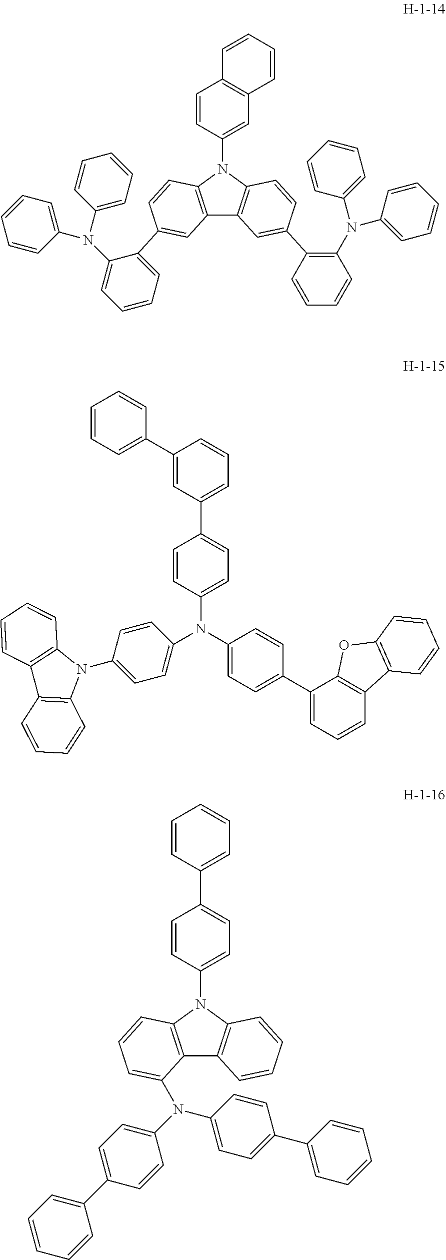 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

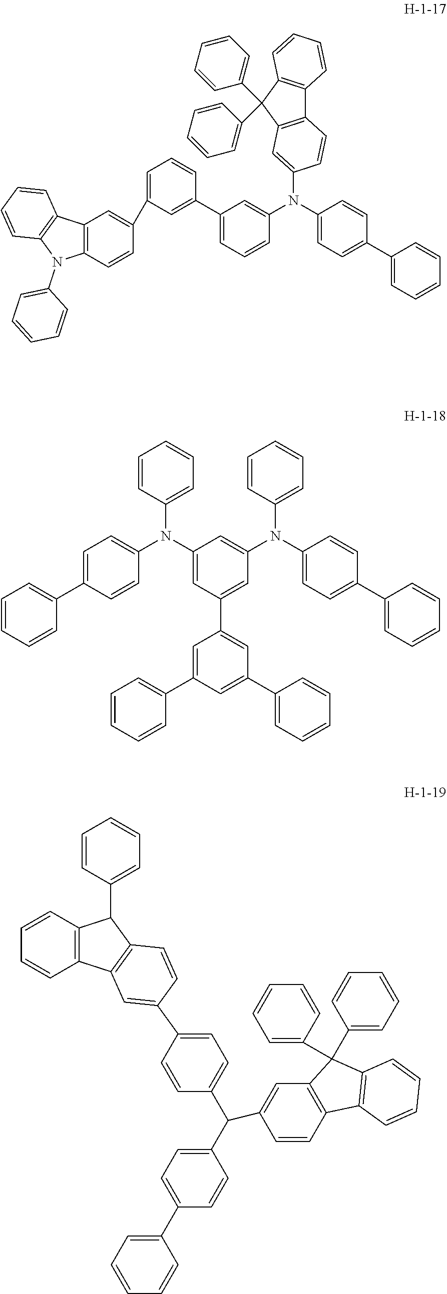

In an embodiment, the hole transport region HTR may further include a suitable hole transport material.

For example, the hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalene-1-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], and/or dipyrazino[2,3-f:2′,3′-h] quinoxaline-2,3,6,7,10,11-hexacarbonitrile (HAT-CN).

The hole transport region HTR may include carbazole derivatives such as N-phenyl carbazole and polyvinyl carbazole, fluorene-based derivatives, triphenylamine-based derivatives such as 4,4′,4″-tris(N-carbazolyl)triphenylamine (TCTA), N,N′-bis(3-methylphenyl)-N,N′-diphenyl-[1,1′-biphenyl]-4,4′-diamine (TPD), N,N′-di(naphthalene-1-yl)-N,N′-diphenyl-benzidine (NPB), 4,4′-cyclohexylidene bis[N,N-bis(4-methylphenyl)benzeneamine] (TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the compounds of the hole transport region in at least one among a hole injection layer HIL, a hole transport layer HTL, and/or an electron blocking layer EBL.

The thickness of the hole transport region HTR may be from about 100 angstrom (Å) to about 10,000 Å, for example, from about 100 Å to about 5,000 Å. When the hole transport region HTR includes a hole injection layer HIL, the thickness of the hole injection region HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL and the electron blocking layer EBL satisfy the above-described ranges, satisfactory hole transport properties may be achieved without substantial increase of a driving voltage.

The hole transport region HTR may further include a charge generating material that is configured to enhance or increase conductivity in addition to the materials described herein. The charge generating material may be dispersed uniformly or non-uniformly in the hole transport region HTR. The charge generating material may be, for example, a p-dopant. The p-dopant may include at least one of metal halide compounds, quinone derivatives, metal oxides, and cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7′,8,8-tetracyanoquinodimethane (F4-TCNQ), metal oxides such as tungsten oxide and molybdenum oxide, cyano group-containing compounds such as dipyrazino[2,3-f: 2′,3′-h] quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but is not limited thereto.

As described elsewhere herein, the hole transport region HTR may further include a buffer layer in addition to the hole injection layer HIL, the hole transport layer HTL and the electron blocking layer EBL. The buffer layer may compensate resonance distance according to the wavelength of light emitted from an emission layer EML and may enhance or increase emission efficiency (e.g., luminance emitting efficiency). The materials included in the buffer layer may utilize materials which may be included in the hole transport region HTR.

The emission layer EML is located or provided on the hole transport region HTR. The emission layer EML may have a thickness of, for example, about 100 Å to about 1,000 Å or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED of an embodiment, the emission layer EML may be configured to emit blue light. The light emitting element ED of an embodiment may include the amine compound of an embodiment in a hole transport region HTR and may show high efficiency (e.g., luminance emitting efficiency) and long lifetime characteristics in a blue emission region. However, an embodiment of the present disclosure is not limited thereto.

In the light emitting element ED of an embodiment, the emission layer EML may include anthracene derivatives, pyrene derivatives, fluoranthene derivatives, chrysene derivatives, dihydrobenzanthracene derivatives, and/or triphenylene derivatives. For example, the emission layer EML may include anthracene derivatives or pyrene derivatives.

In the light emitting elements ED of embodiments, shown in FIG. 3 to FIG. 6, the emission layer EML may include a host and a dopant, and the emission layer EML may include a compound represented by Formula E-1. In an embodiment, the compound represented by Formula E-1 may be utilized as a fluorescence host material.

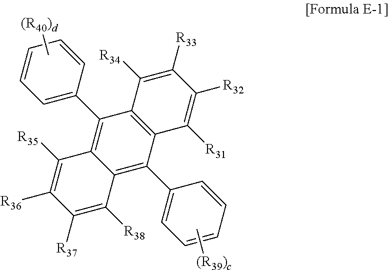

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, c and d may each independently be an integer of 0 to 5.

Formula E-1 may be represented by any one among selected from Compound E1 to Compound E19.

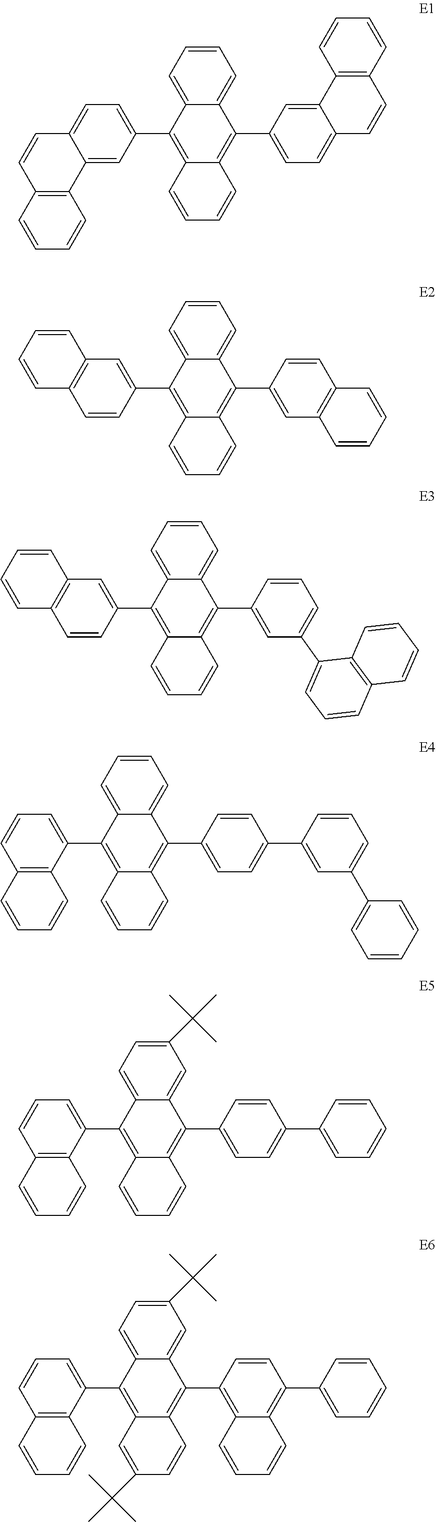

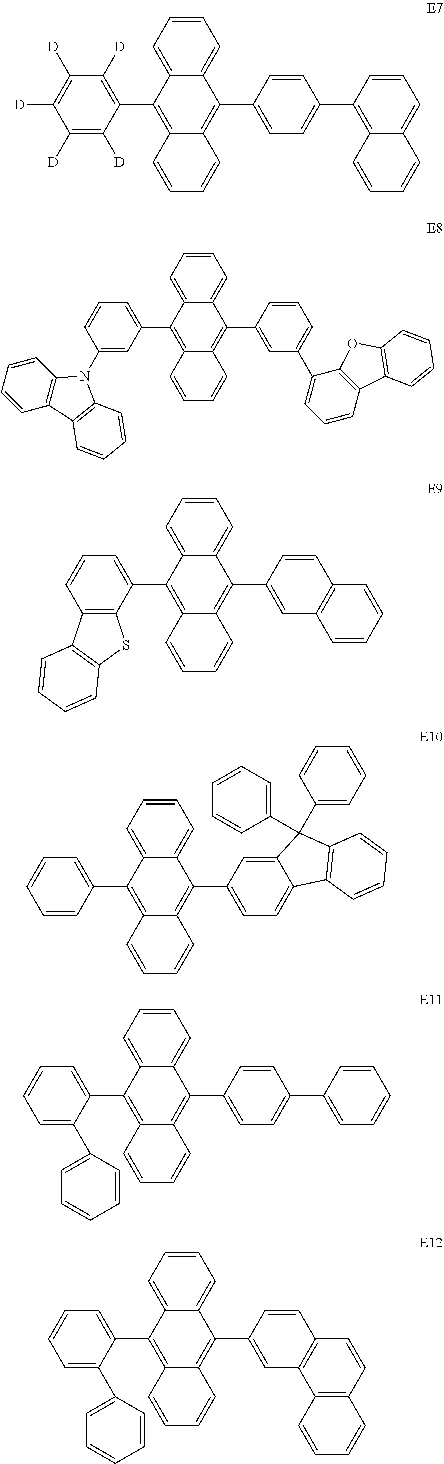

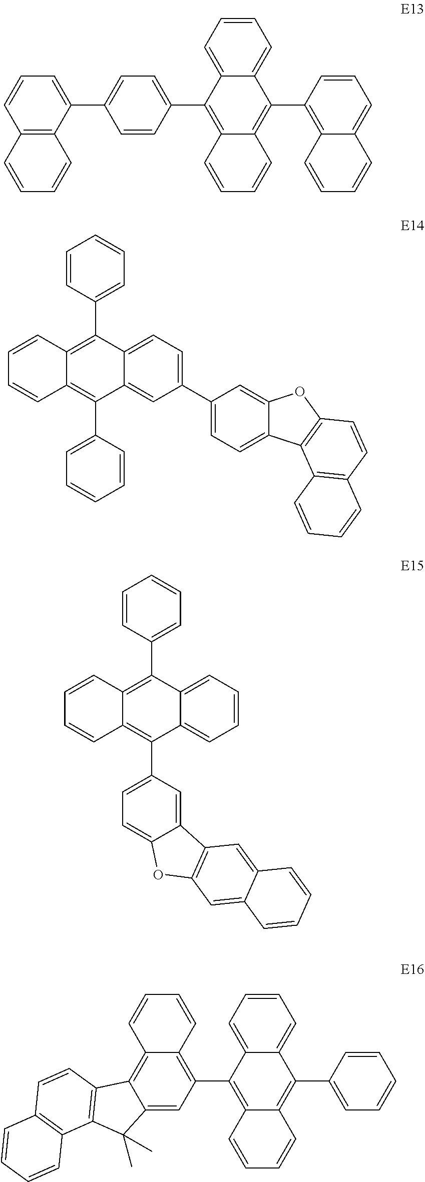

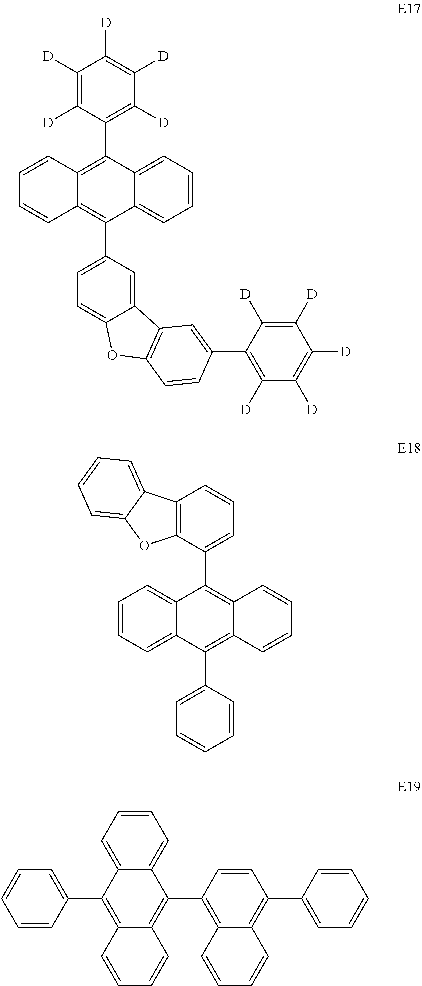

In an embodiment, the emission layer EML may include a compound represented by Formula E-2a or Formula E-2b. In some embodiments, the compound represented by Formula E-2a or Formula E-2b may be utilized as a phosphorescence host material.

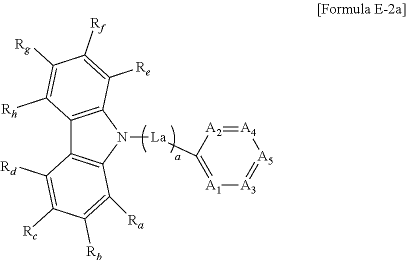

In Formula E-2a, a may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when a is an integer of 2 or more, multiple La may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A5 may each independently be N or CRi. Rₐ to R₁ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. For example, Rₐ to R₁ may be combined with an adjacent group to form a hydrocarbon ring or a heterocycle including N, O, S, etc. as a ring-forming atom.

In some embodiments, in Formula E-2a, two or three selected from A₁ to A5 may be N, and the remainder may be CRI.

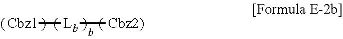

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. In Formula E-2b, b is an integer of 0 to 10, and when b is an integer of 2 or more, multiple L_{b} may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among the compounds in Compound Group E-2. However, the compounds listed in Compound Group E-2 are only illustrations, and the compound represented by Formula E-2a or Formula E-2b is not limited to the compounds represented in Compound Group E-2.

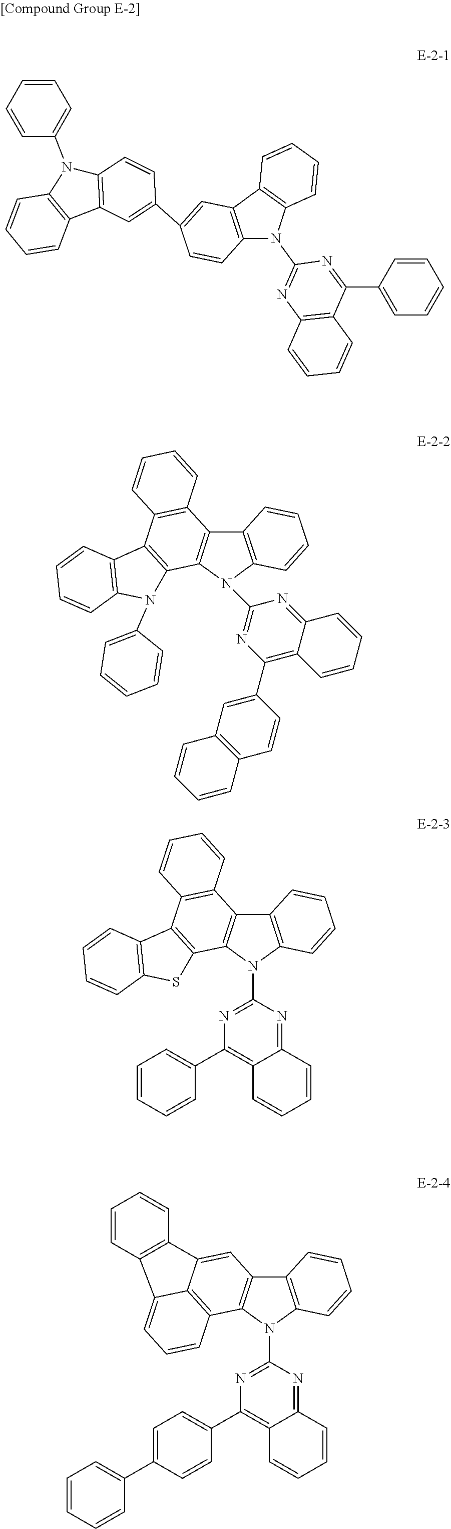

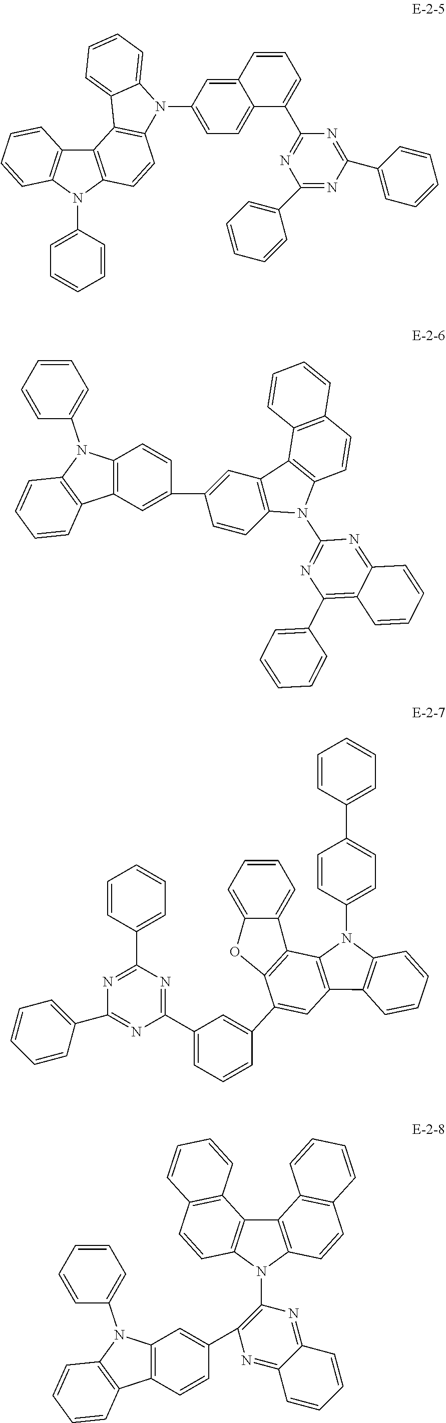 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

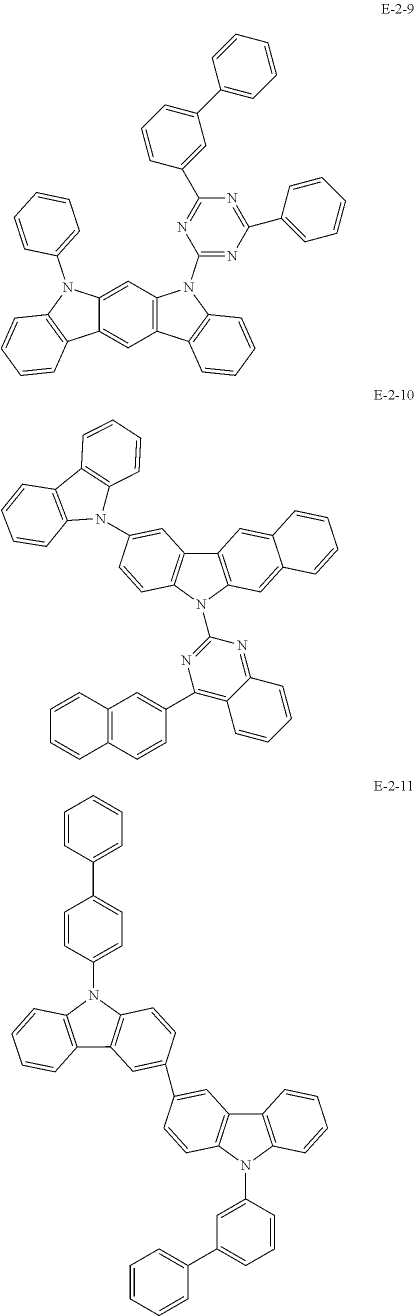

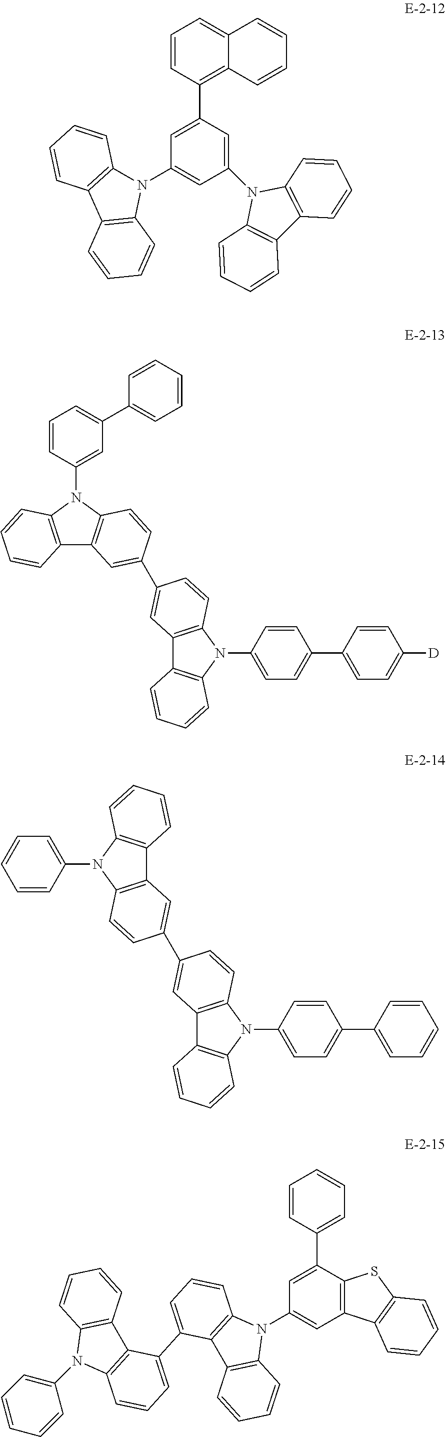 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

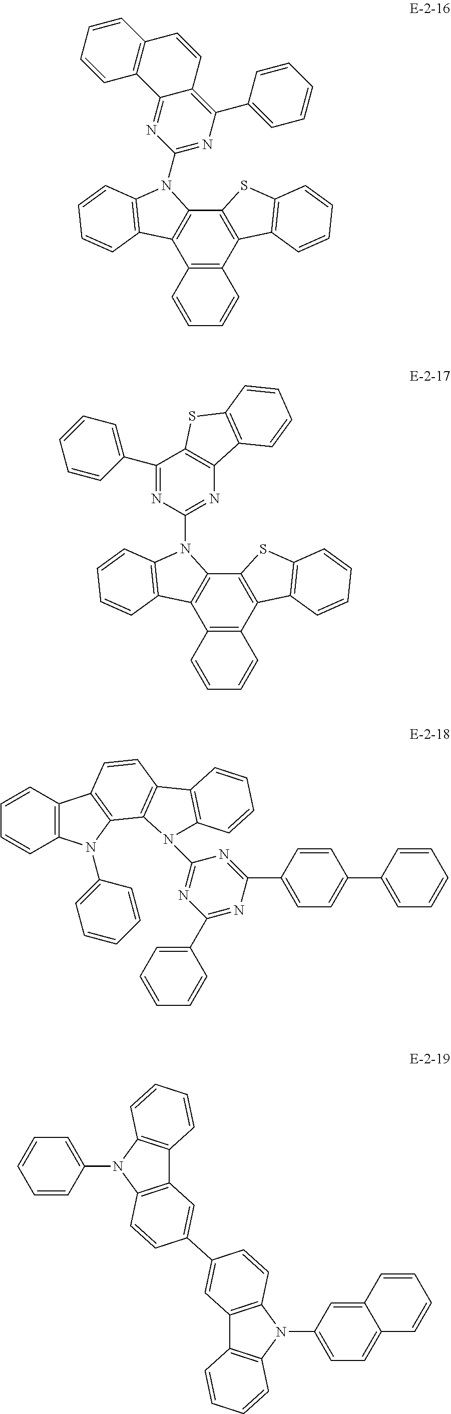 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-n2c3ccccc3c3cc(-c4ccc5c(c4)c4ccccc4n5-c4ccc5ccccc5c4)ccc32)cc1]

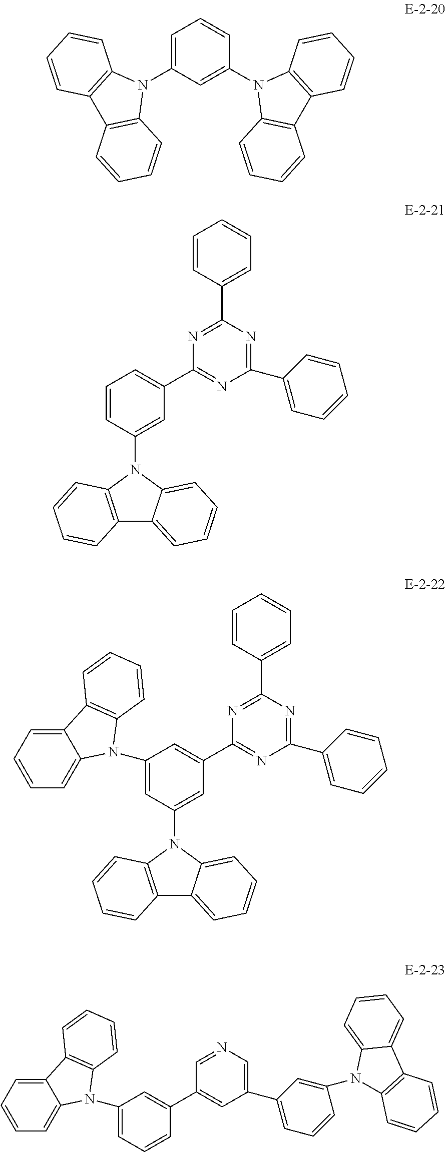 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

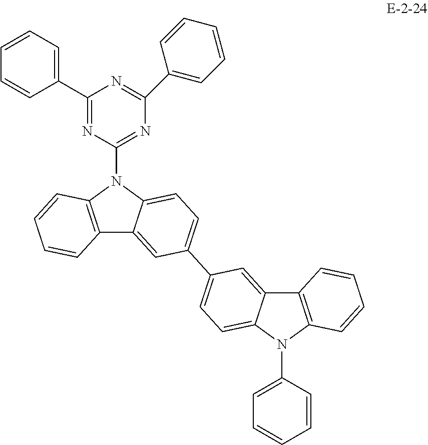 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-suitable in the art as a host material. For example, the emission layer EML may include as a host material, at least one of bis (4-(9H-carbazol-9-yl) phenyl) diphenylsilane (BCPDS), (4-(1-(4-(diphenylamino) phenyl) cyclohexyl) phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, an embodiment of the present disclosure is not limited thereto. For example, tris(8-hydroxyquinolinato)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

The emission layer EML may include a compound represented by Formula M-a or Formula M-b. In an embodiment, the compound represented by Formula M-a or Formula M-b may be utilized as a phosphorescence dopant material. In an embodiment, the compound represented by Formula M-a or Formula M-b may be utilized as an auxiliary dopant material.

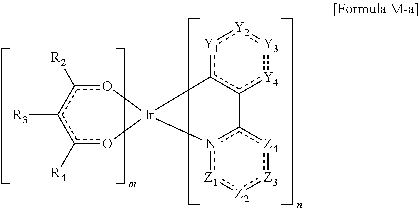

In Formula M-a, Y₁ to Y₄, and Z₁ to Z₄ may each independently be CR₁ or N, and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, m is 0 or 1, and n is 2 or 3. In Formula M-a, when m is 0, then n is 3, and when m is 1, then n is 2.

The compound represented by Formula M-a may be utilized as a phosphorescence dopant.

The compound represented by Formula M-a may be represented by any one selected from among Compounds M-a1 to M-a25. However, Compounds M-a1 to M-a25 are illustrations, and the compound represented by Formula M-a is not limited to the compounds represented b Compounds M-a1 to M-a25.

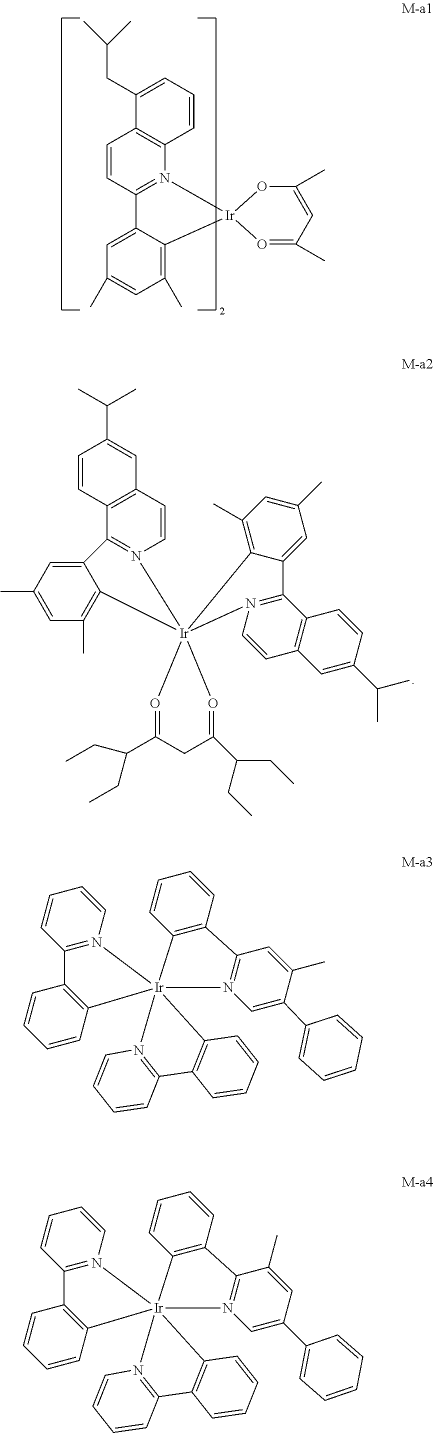

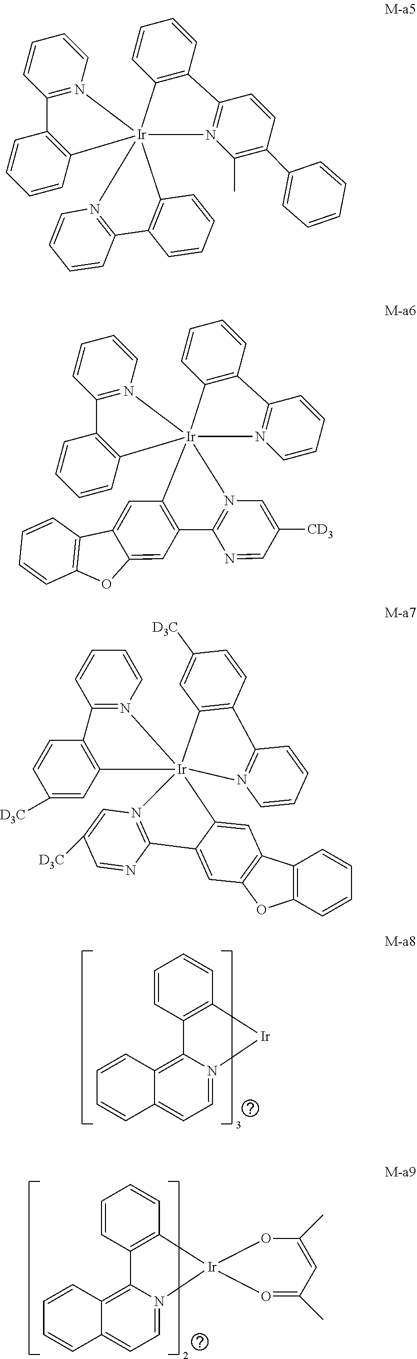

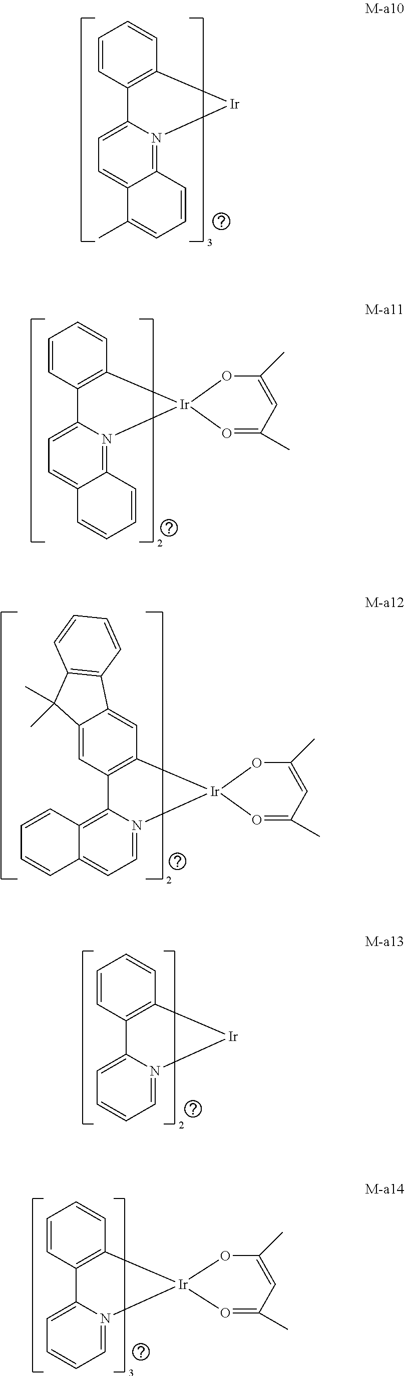

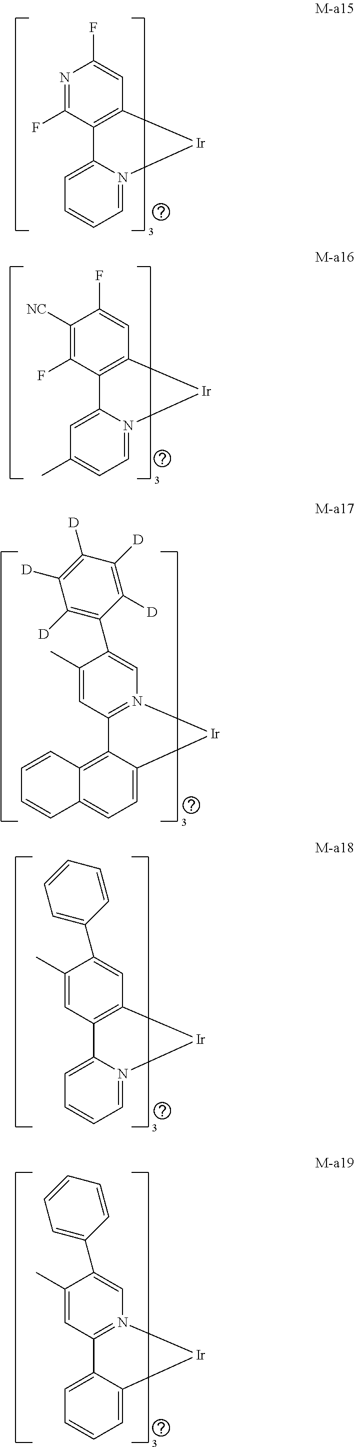

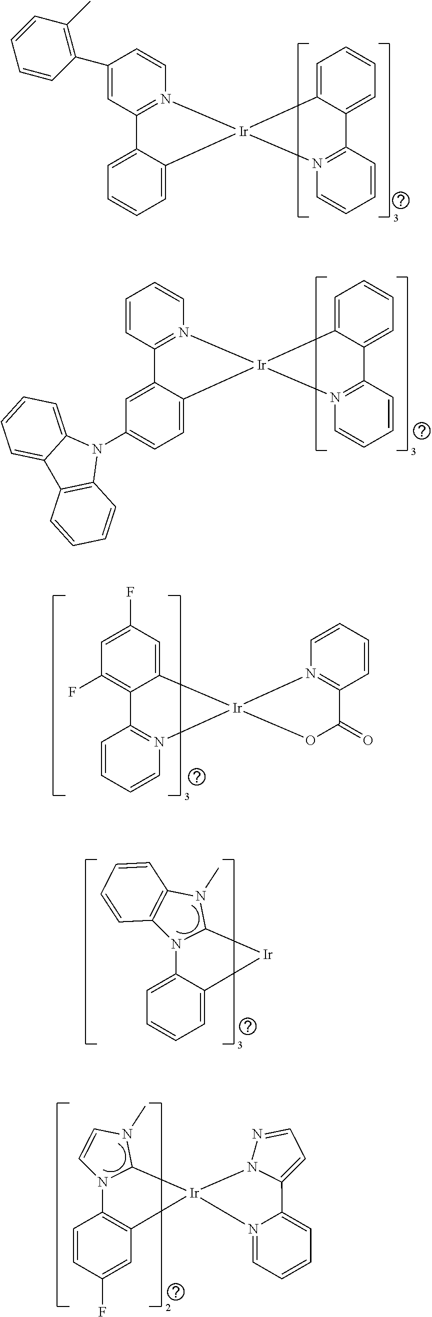

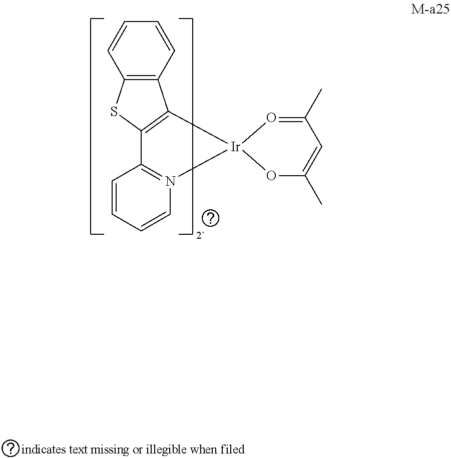

In an embodiment, Compound M-a1 and Compound M-a2 may be utilized as red dopant materials, and Compound M-a3 to Compound M-a7 may be utilized as green dopant materials.

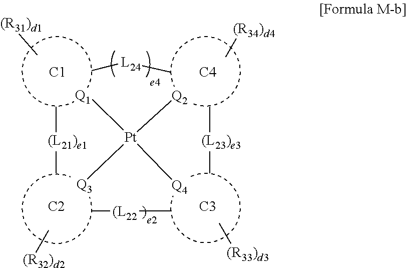

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage, *—O—*, *—S—*,

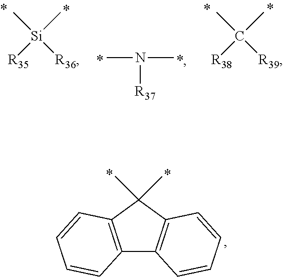

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or bonded to or combined with an adjacent group to form a ring, and d1 to d4 may each independently be an integer of 0 to 4.

In some embodiments, the compound represented by Formula M-b may be utilized as a blue phosphorescence dopant or a green phosphorescence dopant. In some embodiments, the compound represented by Formula M-b may be an auxiliary dopant in an embodiment and may be further included in the emission layer EML.

The compound represented by Formula M-b may be represented by any one selected from among Compound M-b-1 to Compound M-b-11. However, the compounds are illustrations, and the compound represented by Formula M-b is not limited to Compound M-b-1 to Compound M-b-11.

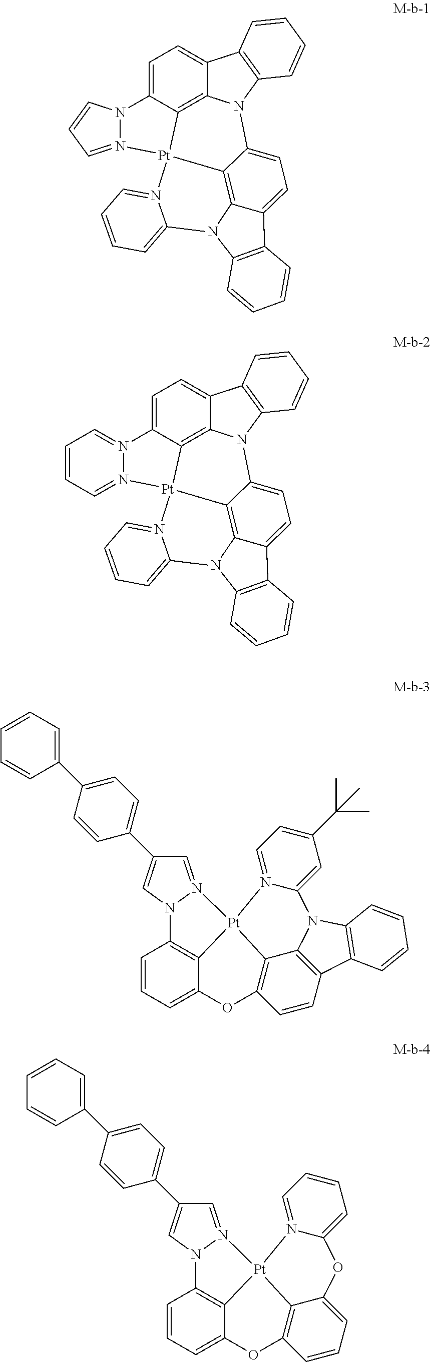

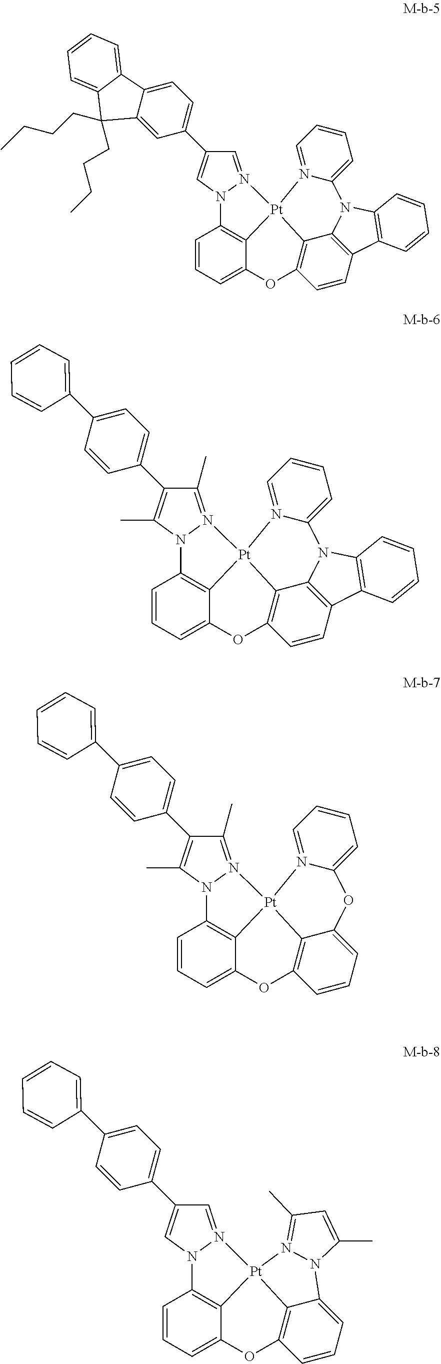

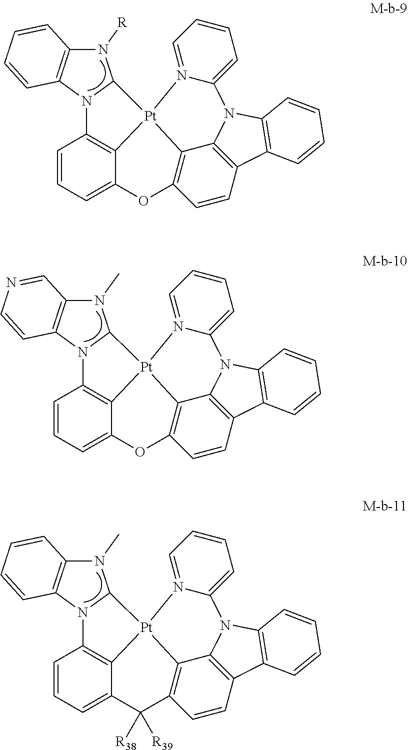

In compounds M-b-9 and M-b-11, R, R₃₈, and R₃₉ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

The emission layer EML may include any one selected from among Formula F-a to Formula F-c. In an embodiment, the compounds represented by Formula F-a to Formula F-c may be utilized as fluorescence dopant materials.

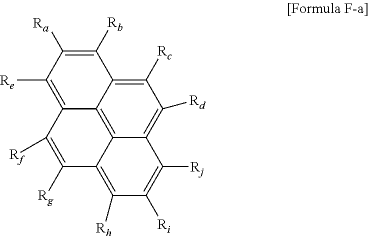

In Formula F-a, two selected from Rₐ to Rⱼ may each independently be substituted with *—NAr₁Ar₂. The remainder not substituted with *—NAr₁Ar₂ among Rₐ to Rⱼ may each independently be a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

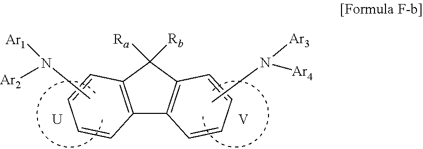

In Formula F-b, Rₐ and R_{b} may each independently be a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring.

In Formula F-b, Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the value of U or V is 1, one ring forms a fused ring at the designated part by U or V, and when the value of U or V is 0, a ring is not present at the designated part by U or V. For example, when the value of U is 0, and the value of V is 1, or when the value of U is 1, and the value of V is 0, a fused ring having the fluorene core of Formula F-b may be a ring compound with four rings. In some embodiments, when the value of both (e.g., simultaneously) U and V is 0, the fused ring of Formula F-b may be a ring compound with three rings. In some embodiments, when the value of both (e.g., simultaneously) U and V is 1, a fused ring having the fluorene core of Formula F-b may be a ring compound with five rings.

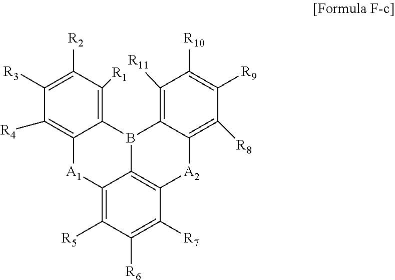

In Formula F-c, A₁ and A₂ may each independently be O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may each independently be combined with the substituents of an adjacent ring to form a fused ring. For example, when A₁ and A₂ may each independently be NRₘ, A₁ may be combined with R₄ or R₅ to form a ring. In some embodiments, A₂ may be combined with R₇ or R₈ to form a ring.

In an embodiment, the emission layer EML may include as a suitable dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), and/or the like.

In an embodiment, when multiple emission layers EML are included, at least one emission layer EML may include a suitable phosphorescence dopant material. For example, the phosphorescence dopant may utilize a metal complex including iridium (Ir), platinum (Pt), osmium (Os), gold (Au), titanium (Ti), zirconium (Zr), hafnium (Hf), europium (Eu), terbium (Tb) and/or thulium (Tm). For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), and/or platinum octaethyl porphyrin (PtOEP) may be utilized as the phosphorescence dopant. However, an embodiment of the present disclosure is not limited thereto.

In an embodiment, the emission layer EML may include a hole transport host and an electron transport host. In some embodiments, the emission layer EML may include an auxiliary dopant and a light emitting dopant. In some embodiments, the auxiliary dopant may include a phosphorescence dopant material or a thermally activated delayed fluorescence dopant. For example, in an embodiment, the emission layer EML may include a hole transport host, an electron transport host, an auxiliary dopant, and a light emitting dopant.

In some embodiments, an exciplex may be formed by the hole transport host and the electron transport host in the emission layer EML. In this case, the triplet energy of the exciplex formed by the hole transport host and the electron transport host may correspond to T1 which is a gap between the LUMO energy level of the electron transport host and the HOMO energy level of the hole transport host.

In an embodiment, the triplet energy (T1) of the exciplex formed by the hole transport host and the electron transport host may be about 2.4 eV to about 3.0 eV. In some embodiments, the triplet energy of the exciplex may be a value less than or smaller than the energy gap of each host material. Accordingly, the exciplex may have a triplet energy of about 3.0 eV or less, which is the energy gap between the hole transport host and the electron transport host.

In some embodiments, at least one emission layer EML may include a quantum dot material. The core of the quantum dot may be selected from II-VI group compounds, III-VI group compounds, I-III-VI group compounds, III-V group compounds, III-II-V group compounds, IV-VI group compounds, IV group elements, IV group compounds, and/or combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

The III-VI group compound may include a binary compound such as In₂S₃ and/or In₂Se₃, a ternary compound such as InGaS₃ and/or InGaSe₃, or optional combination(s) thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, and/or a quaternary compound such as AgInGaS₂ and/or CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and mixtures thereof. In some embodiments, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In some embodiments, the binary compound, the ternary compound or the quaternary compound may be present at substantially uniform concentration in a particle or may be present at a partially different concentration distribution state in substantially the same particle. In some embodiments, a core/shell structure in which one quantum dot wraps another quantum dot may be possible. The interface of the core and the shell may have a concentration gradient in which the concentration of an element present in the shell is decreased toward the core or center.

In some embodiments, the quantum dot may have the described core-shell structure including a core including a nanocrystal and a shell wrapping around the core. The shell of the quantum dot may be configured to be (or play the role of) a protection layer for preventing or reducing the chemical deformation of the core to maintain or enhance the semiconductor properties and/or a charging layer for imparting or configuring the quantum dot with electrophoretic properties. The shell may have a single layer or a multilayer. Examples of the shell of the quantum dot may include a metal or non-metal oxide, a semiconductor compound, and/or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and/or NiO, or a ternary compound such as MgAl₂O₄, CoFe₂O₄, NiFe₂O₄ and/or CoMn₂O₄, but an embodiment of the present disclosure is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and/or the like, but an embodiment of the present disclosure is not limited thereto.

The quantum dot may have a full width of half maximum (FWHM) of emission wavelength spectrum of about 45 nm or less, , about 40 nm or less, more, about 30 nm or less. Within this range, color purity and/or color reproducibility may be enhanced or improved. In some embodiments, light emitted by or via such quantum dot is emitted in all directions, and the light viewing angle properties of a display device and/or light emitting element having the quantum dot as described herein may be enhanced or improved (e.g., the size or width of the viewing angle may be enhanced or increased).

In some embodiments, the shape of the quantum dot may be generally utilized shapes in the art, without specific limitation. In some embodiments, the shape of spherical, pyramidal, multi-arm, or cubic nanoparticle, nanotube, nanowire, nanofiber, nanoplate particle, etc. may be utilized.

The quantum dot may determine or control the color of light emitted according to the particle size, and accordingly, the quantum dot may have one or more suitable emission colors such as blue, red, green, and/or the like.

In the light emitting elements ED of embodiments, as shown in FIG. 3 to FIG. 6, the electron transport region ETR is located or provided on the emission layer EML. The electron transport region ETR may include at least one of a hole blocking layer HBL, an electron transport layer ETL or an electron injection layer EIL. However, an embodiment of the present disclosure is not limited thereto.

The electron transport region ETR may have a single layer formed utilizing a single material, a single layer formed utilizing multiple different materials, and/or a multilayer structure having multiple layers formed utilizing multiple different materials.

For example, the electron transport region ETR may have a single layer structure of an electron injection layer EIL or an electron transport layer ETL, or a single layer structure formed utilizing an electron injection material and an electron transport material. Further, the electron transport region ETR may have a single layer structure formed utilizing multiple different materials, and/or a structure stacked from the emission layer EML of electron transport layer ETL/electron injection layer EIL, hole blocking layer HBL/electron transport layer ETL/electron injection layer EIL, electron transport layer ETL/buffer layer/electron injection layer EIL, without limitation. The thickness of the electron transport region ETR may be, for example, from about 1,000 Å to about 1,500 Å.

The electron transport region ETR may be formed utilizing one or more suitable methods such as a vacuum deposition method, a spin coating method, a cast method, a Langmuir-Blodgett (LB) method, an inkjet printing method, a laser printing method, and/or a laser induced thermal imaging (LITI) method.

The electron transport region ETR may include a compound represented by Formula ET-1.

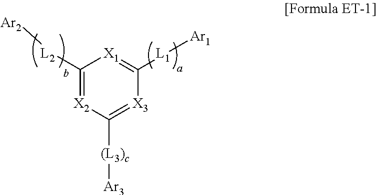

In Formula ET-1, at least one among X₁ to X₃ is N, and the remainder are CRₐ. Each Rₐ may independently be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-1, a to c may each independently be an integer of 0 to 10. In Formula ET-1, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when a to c are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, an embodiment of the present disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and mixtures thereof, without limitation.

The electron transport region ETR may include at least one selected from among Compounds ET1 to ET36.

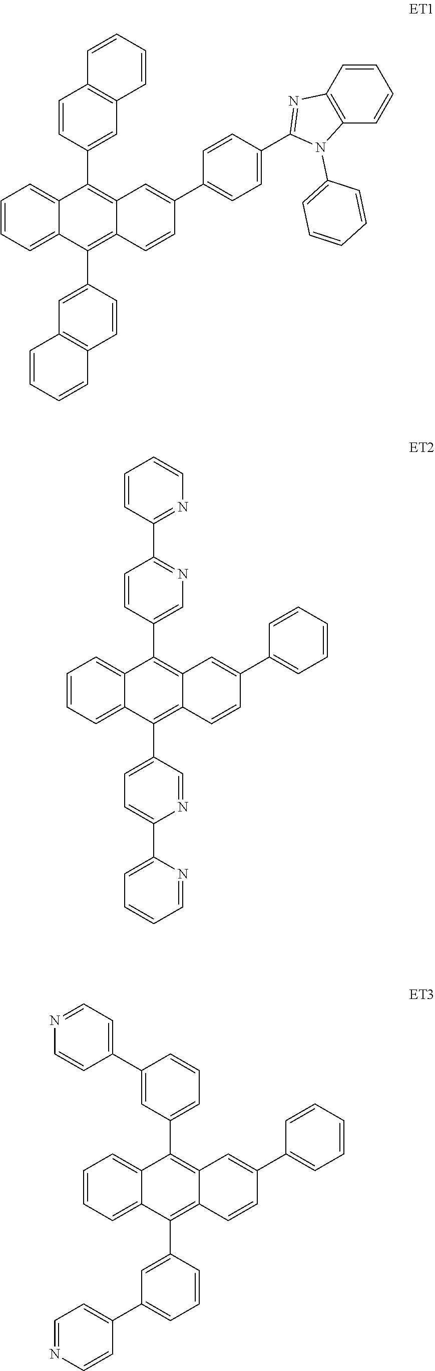 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

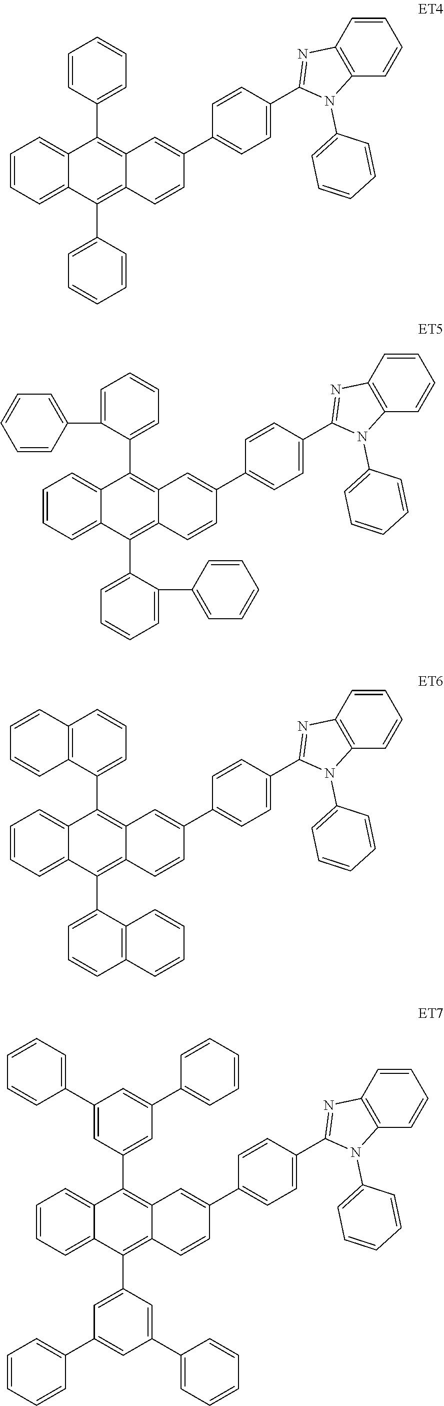 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

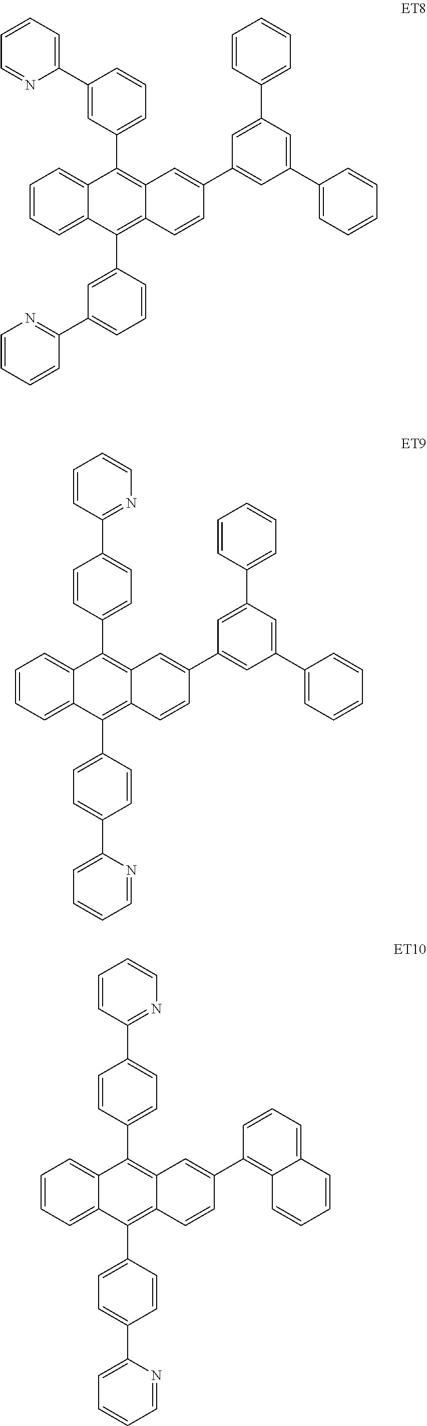

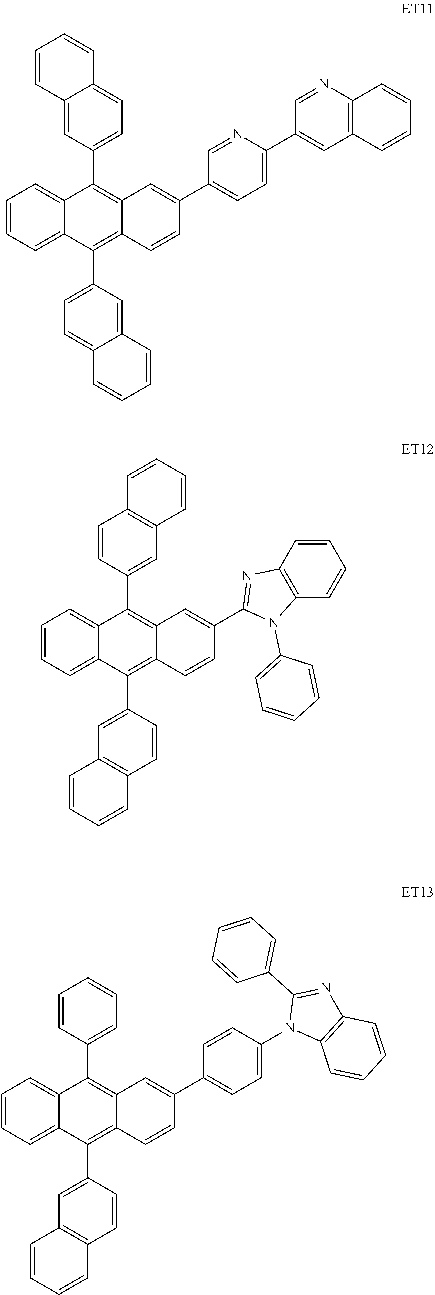 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

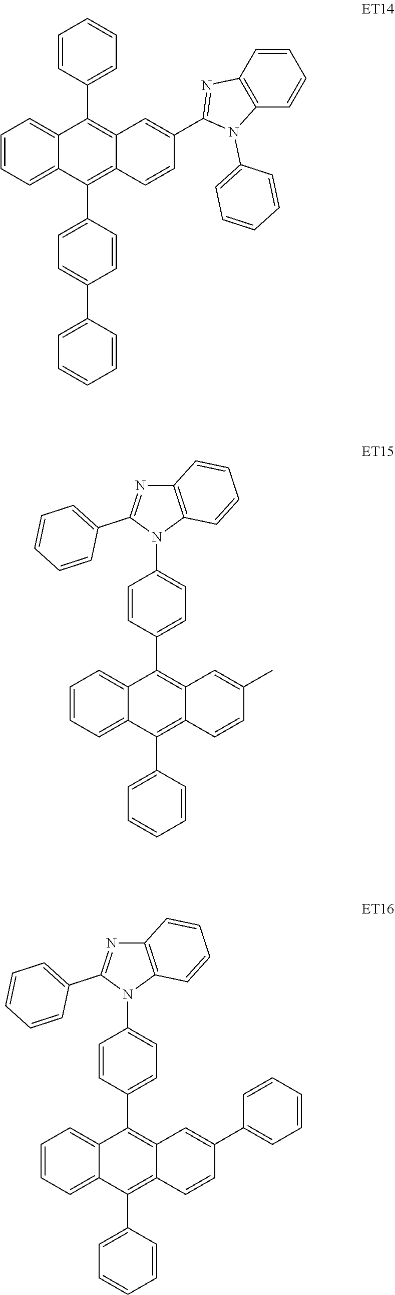 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

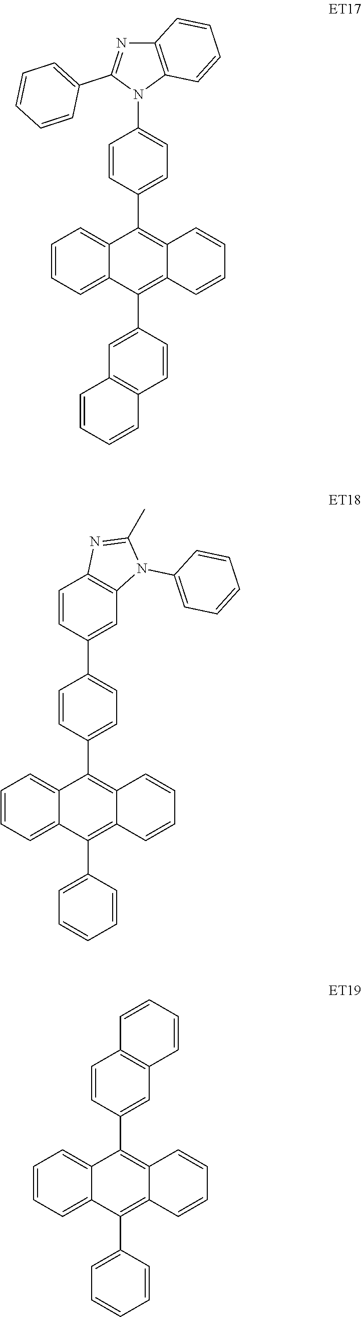

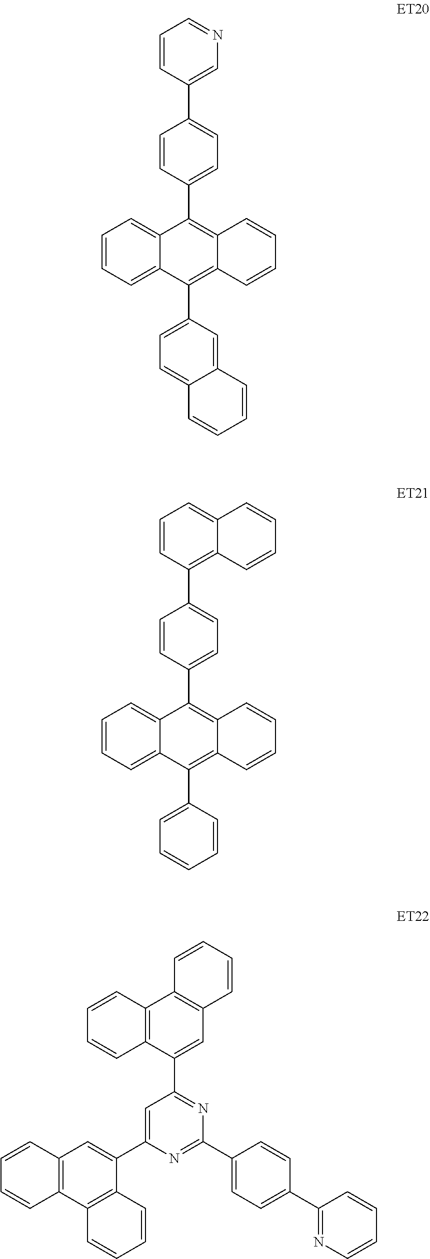

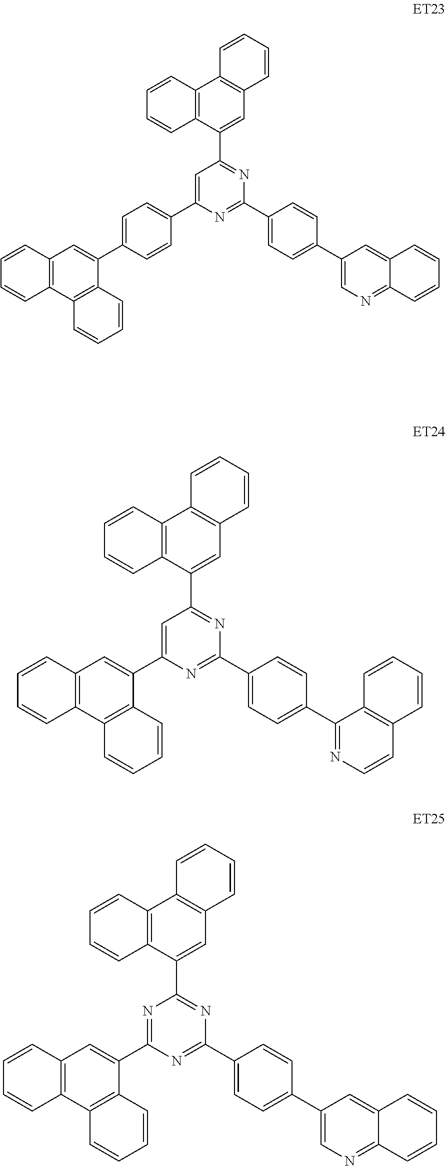 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

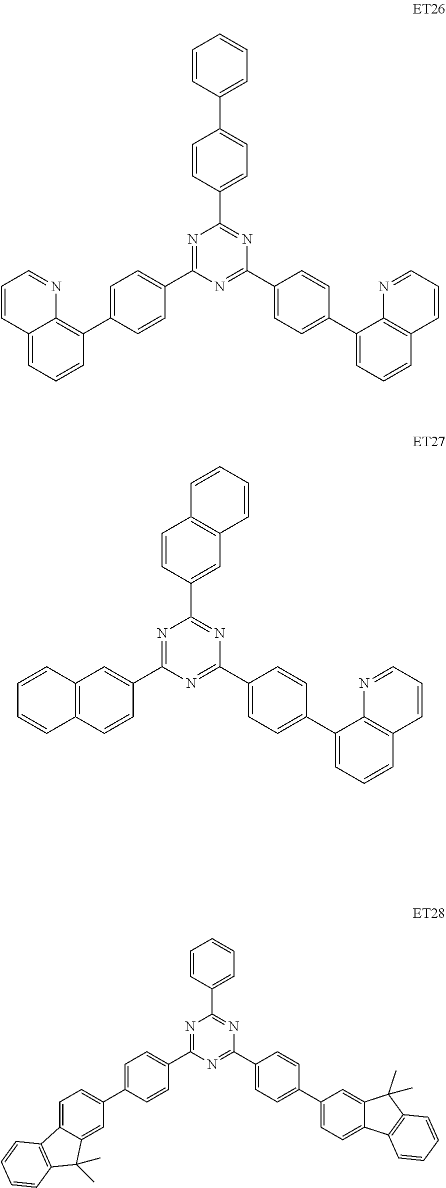 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

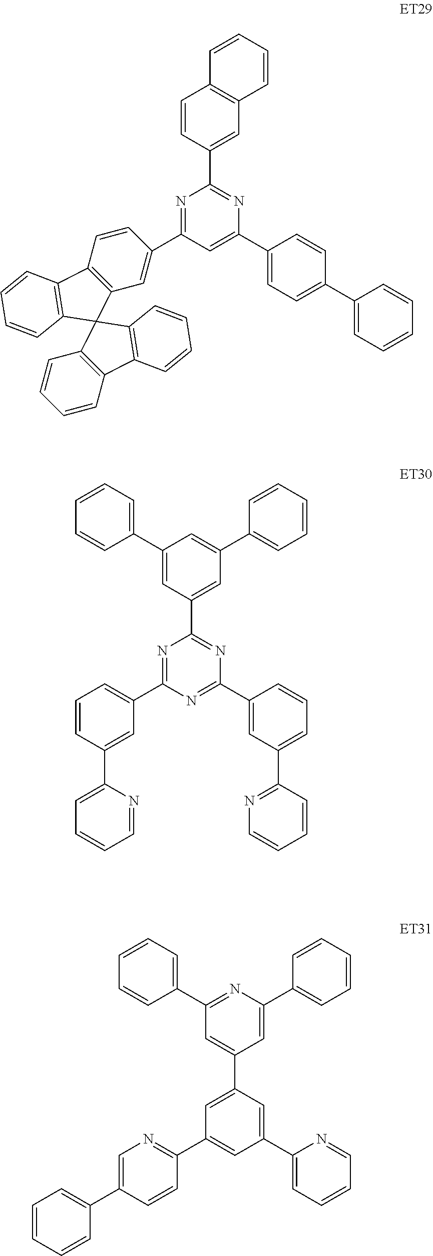 [SMILES: c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1]

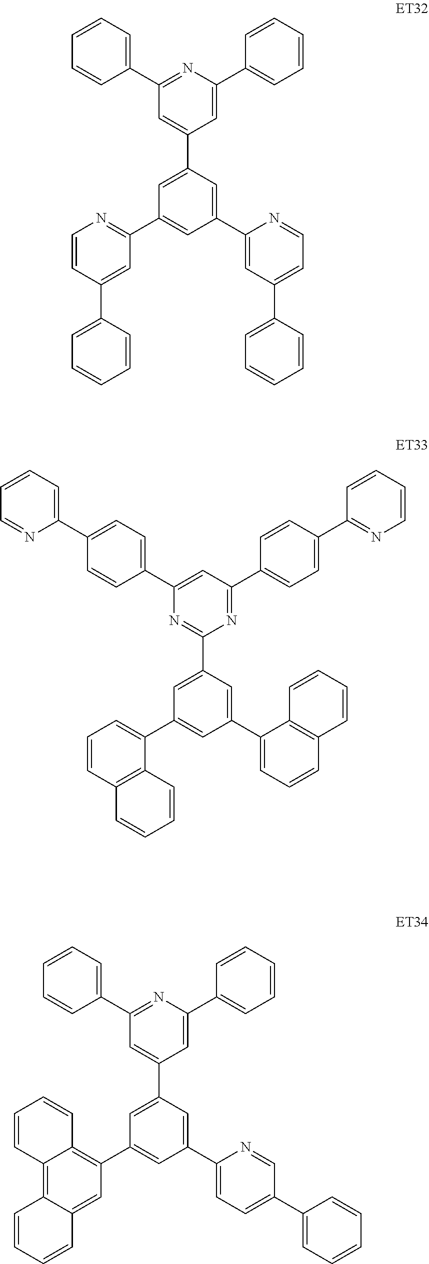 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

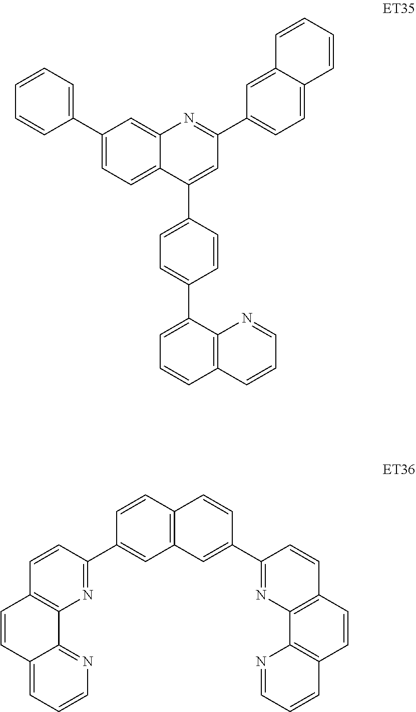 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, KI, and/or the like, a lanthanide metal such as Yb, and/or the like, or a co-depositing material of the metal halide and the lanthanide metal. For example, the electron transport region ETR may include KI:Yb, RbI:Yb, LiF:Yb, and/or the like, as the co-depositing material. In some embodiments, the electron transport region ETR may utilize a metal oxide such as Li₂O and BaO, or 8-hydroxy-lithium quinolate (Liq). However, an embodiment of the present disclosure is not limited thereto. The electron transport region ETR also may be formed utilizing a mixture material of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, metal stearates, and/or the like.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, an embodiment of the present disclosure is not limited thereto.

The electron transport region ETR may include the compounds of the electron transport region in at least one among an electron injection layer EIL, an electron transport layer ETL, and a hole blocking layer HBL.

When the electron transport region ETR includes the electron transport layer ETL, the thickness of the electron transport layer ETL may be from about 100 Å to about 1,000 Å, for example, from about 150 Å to about 500 Å. When the thickness of the electron transport layer ETL satisfies the described range, satisfactory electron transport properties may be obtained without substantial increase of a driving voltage. When the electron transport region ETR includes the electron injection layer EIL, the thickness of the electron injection layer EIL may be from about 1 Å to about 100 Å, and from about 3 Å to about 90 Å. When the thickness of the electron injection layer EIL satisfies the described range, satisfactory electron injection properties may be obtained without inducing substantial increase of a driving voltage.

The second electrode EL2 is located or provided on the electron transport region ETR. The second electrode EL2 may be a common electrode. The second electrode EL2 may be a cathode or an anode, but an embodiment of the present disclosure is not limited thereto. For example, when the first electrode EL1 is an anode, the second cathode EL2 may be a cathode, and when the first electrode EL1 is a cathode, the second electrode EL2 may be an anode. The second electrode EL2 may include at least one selected among Ag, Mg, Cu, Al, Pt, Pd, Au, Ni, Nd, Ir, Cr, Li, Ca, LiF, Mo, Ti, W, In, Sn, Zn, compounds of two or more selected therefrom, mixtures of two or more selected therefrom, and/or oxides thereof.

The second electrode EL2 may be a transmissive electrode, a transflective electrode or a reflective electrode. When the second electrode EL2 is the transmissive electrode, the second electrode EL2 may include a transparent metal oxide, for example, ITO, IZO, ZnO, ITZO, and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, Yb, W, compound(s) including thereof, or mixture(s) thereof (for example, AgMg, AgYb, or MgAg). In some embodiments, the second electrode EL2 may have a multilayered structure including a reflective layer or a transflective layer formed utilizing the above-described materials and a transparent conductive layer formed utilizing ITO, IZO, ZnO, ITZO, etc. For example, the second electrode EL2 may include the aforementioned metal material(s), combination(s) of two or more metal materials selected from the aforementioned metal materials, or oxide(s) of the aforementioned metal materials.

In some embodiments, the second electrode EL2 may be connected to or with an auxiliary electrode. When the second electrode EL2 is connected to or with the auxiliary electrode, the resistance of the second electrode EL2 may decrease.

In some embodiments, on the second electrode EL2 in the light emitting element ED of an embodiment, a capping layer CPL may be further located or disposed. The capping layer CPL may include a multilayer or a single layer.

In an embodiment, the capping layer CPL may be an organic layer or an inorganic layer. For example, when the capping layer CPL includes an inorganic material, the inorganic material may include an alkali metal compound such as LiF, and/or an alkaline earth metal compound such as SiON, SiNx, SiOy, and/or the like.

For example, when the capping layer CPL includes an organic material, the organic material may include 2,2′-dimethyl-N,N′-di-[(1-naphthyl)-N,N′-diphenyl]-1,1′-biphenyl-4,4′-diamine(a-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl) triphenylamine (TCTA), and/or the like, or may include an epoxy resin and/or acrylate such as methacrylate. In some embodiments, a capping layer CPL may include at least one selected from among Compounds P1 to P5, but an embodiment of the present disclosure is not limited thereto.

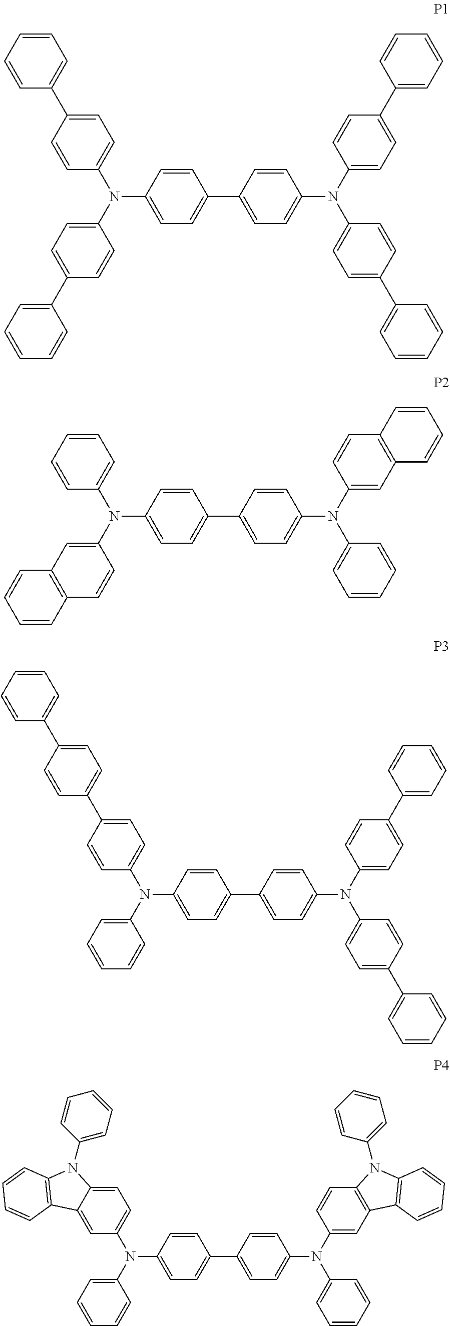 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

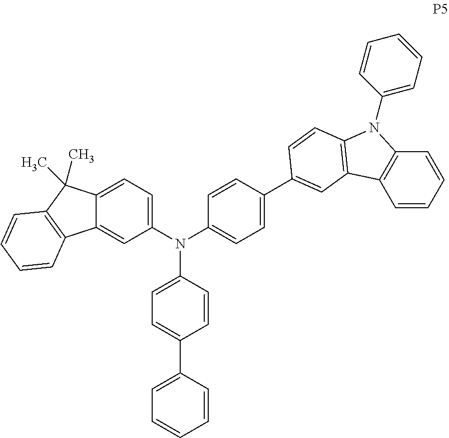 [SMILES: CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, the refractive index of the capping layer CPL with respect to light in a wavelength range of about 550 nm to about 660 nm may be about 1.6 or more.

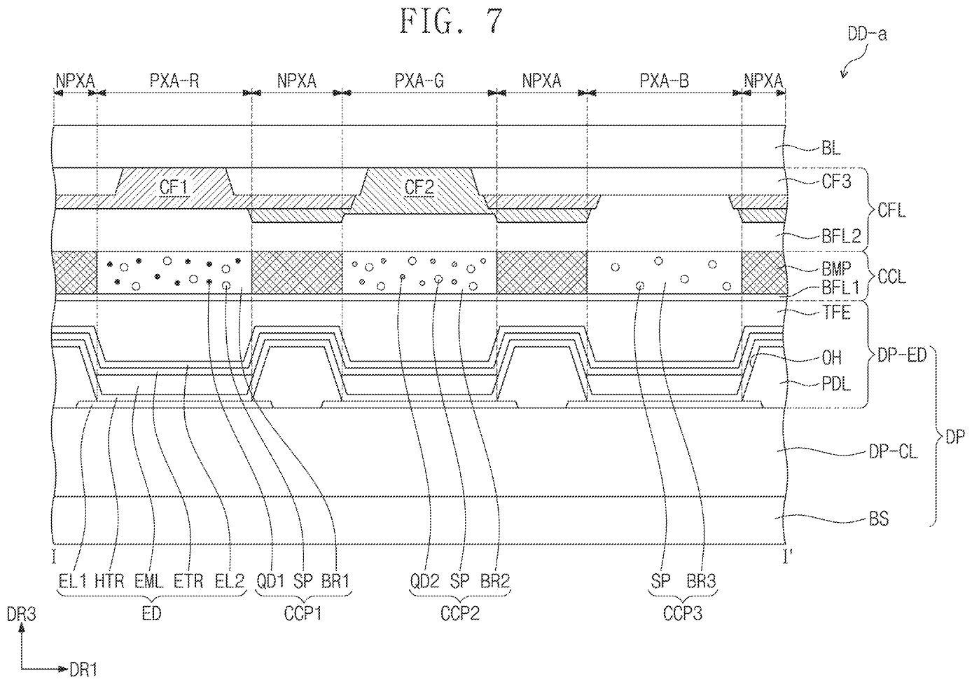

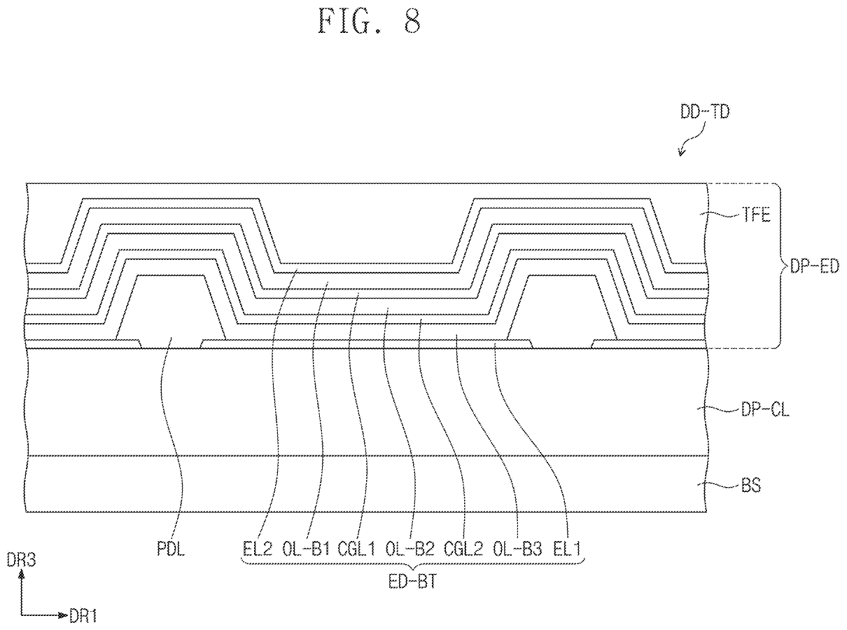

FIG. 7 to FIG. 10 are each a cross-sectional view of a display device according to an embodiment. Hereinafter, in the explanation of the display devices of the embodiments, referring to FIG. 7 to FIG. 10, parts overlapping with the explanation on FIG. 1 to FIG. 6 will not be explained again, and, instead, the different features will be explained chiefly.

Referring to FIG. 7, a display device DD-a according to an embodiment may include a display panel DP including a display element layer DP-ED, a light controlling layer CCL located or disposed on the display panel DP, and a color filter layer CFL.

In an embodiment shown in FIG. 7, the display panel DP includes a base layer BS, a circuit layer DP-CL located or provided on the base layer BS and a display element layer DP-ED, and the display element layer DP-ED may include a light emitting element ED.

The light emitting element ED may include a first electrode EL1, a hole transport region HTR located or disposed on the first electrode EL1, an emission layer EML located or disposed on the hole transport region HTR, an electron transport region ETR disposed on the emission layer EML, and a second electrode EL2 located or disposed on the electron transport region ETR. In some embodiments, the structures of the light emitting elements of FIG. 3 to FIG. 6 may be applied to the structure of the light emitting element ED shown in FIG. 7.

The hole transport region HTR of the light emitting element ED included in the display device DD-a according to an embodiment may include the amine compound of an embodiment.

Referring to FIG. 7, the emission layer EML may be located or disposed in an opening part OH defined in a pixel definition layer PDL. For example, the emission layer EML that is divided by the pixel definition layer PDL and correspondingly located or provided to each of luminous areas PXA-R, PXA-G and PXA-B may be configured to emit light in substantially the same wavelength region. In the display device DD-a of an embodiment, the emission layer EML may be configured to emit blue light. In some embodiments, different from the drawings, in an embodiment, the emission layer EML may be located or provided as a common layer for all luminous areas PXA-R, PXA-G and PXA-B.

The light controlling layer CCL may be located or disposed on the display panel DP. The light controlling layer CCL may include a light converter. The light converter may be a quantum dot or a phosphor. The light converter may transform the wavelength of light incident (e.g., incoming or provided) and then emit the light having a transformed wavelength. For example, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be located or disposed between the separated light controlling parts CCP1, CCP2 and CCP3, but an embodiment of the present disclosure is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In an embodiment, the first light controlling part CCP1 may emit or provide red light which is the second color light, and the second light controlling part CCP2 may emit or provide green light which is the third color light. The third color controlling part CCP3 may be configured to transmit and emit or provide blue light which is the first color light emitted or provided from the light emitting element ED. For example, the first quantum dot QD1 may be a red quantum dot, and the second quantum dot QD2 may be a green quantum dot. On the quantum dots QD1 and QD2, the same contents as those described herein may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a (any) quantum dot but include the scatterer SP.

The scatterer SP may be an inorganic particle. For example, the scatterer SP may include at least one selected from among TiO₂, ZnO, Al₂O₃, SiO₂, and hollow silica. The scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are each a composition or medium in which the quantum dots QD1 and QD2 and the scatterer SP are dispersed, and may be composed of one or more suitable resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may be located or disposed on the light controlling parts CCP1, CCP2 and CCP3 and may block or reduce the exposure of the light controlling parts CCP1, CCP2 and CCP3 to humidity/oxygen. In some embodiments, the barrier layer BFL1 may cover the light controlling parts CCP1, CCP2 and CCP3. In some embodiments, the barrier layer BFL2 may be located or provided between the light controlling parts CCP1, CCP2 and CCP3 and a color filter layer CFL.

The barrier layers BFL1 and BFL2 may include at least one inorganic layer. For example, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and/or silicon oxynitride, or a metal thin film configured to secure light transmittance. In some embodiments, the barrier layers BFL1 and BFL2 may further include an organic layer. The barrier layers BFL1 and BFL2 may include or be composed of a single layer of multiple layers.

In the display device DD-a of an embodiment, the color filter layer CFL may be located or disposed on the light controlling layer CCL. For example, the color filter layer CFL may be located or disposed directly on the light controlling layer CCL. In this case, the barrier layer BFL2 may not be provided.

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and/or a pigment and/or a dye. The first filter CF1 may include a red pigment or and/dye, the second filter CF2 may include a green pigment and/or dye, and the third filter CF3 may include a blue pigment and/or dye. In some embodiments, an embodiment of the present disclosure is not limited thereto, and the third filter CF3 may not include (e.g., may exclude (e.g., exclude any of)) the pigment and/or dye. The third filter CF3 may include a polymer photosensitive resin and not include a (any) pigment and/or dye. The third filter CF3 may be transparent. The third filter CF3 may be formed utilizing a transparent photosensitive resin.

In some embodiments, in an embodiment, the first filter CF1 and/or the second filter CF2 may be yellow filters. The first filter CF1 and/or the second filter CF2 may be located or provided in one body without distinction. The first to third filters CF1, CF2 and CF3 may be located or disposed corresponding to the red luminous area PXA-R, the green luminous area PXA-G and the blue luminous area PXA-B, respectively.

In some embodiments, the color filter layer CFL may further include a light blocking part. The color filter layer CFL may include a light blocking part located or disposed at the boundaries to overlap with adjacent filters CF1, CF2 and CF3. The light blocking part may be a black matrix. The light blocking part may be formed by including an organic light blocking material or an inorganic light blocking material, including a black pigment and/or black dye. The light blocking part may divide or separate the boundaries among adjacent filters CF1, CF2 and CF3. In some embodiments, in an embodiment, the light blocking part may be formed as a blue filter.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, an embodiment of the present disclosure is not limited thereto, and the base substrate BL may be an inorganic layer, an organic layer or a composite material layer. In some embodiments, different from the drawing, the base substrate BL may not be provided in an embodiment.

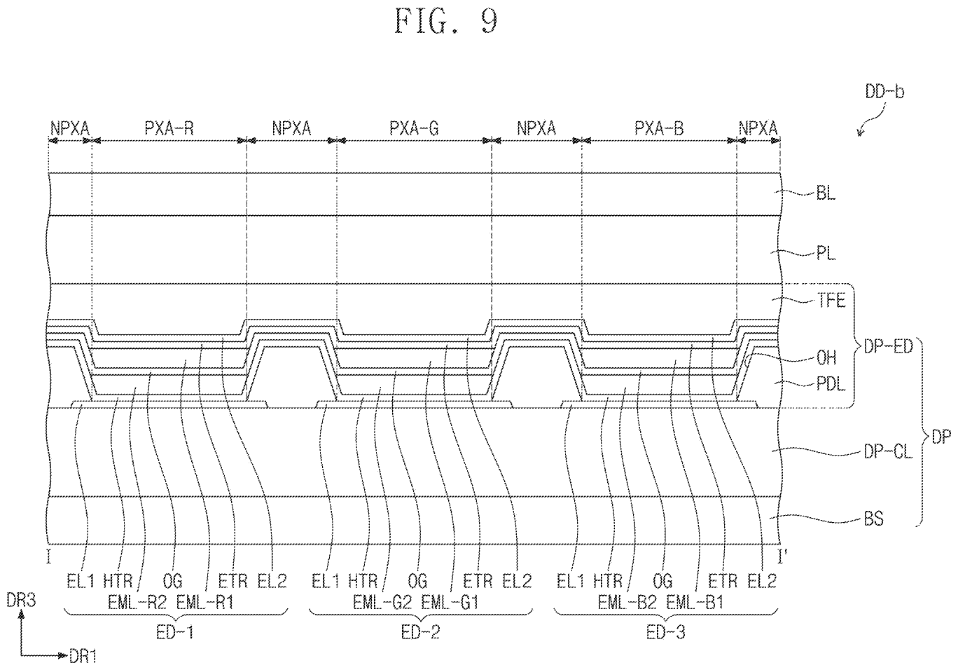

Referring to FIG. 8, a display device DD-TD of an embodiment includes the light emitting element ED-BT that may include multiple light emitting structures OL-B1, OL-B2 and OL-B3. The light emitting element ED-BT may include oppositely disposed first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include the emission layer EML (FIG. 7), and a hole transport region HTR and an electron transport region ETR, located or disposed with the emission layer EML (FIG. 7) therebetween.

For example, the light emitting element ED-BT included in the display device DD-TD of an embodiment may be a light emitting element of a tandem structure including multiple emission layers.

In an embodiment shown in FIG. 8, light emitted from the light emitting structures OL-B1, OL-B2 and OL-B3 may be all blue light. However, an embodiment of the present disclosure is not limited thereto, and the wavelength regions of light emitted from the light emitting structures OL-B1, OL-B2 and OL-B3 may be different from each other. For example, the light emitting element ED-BT including the multiple light emitting structures OL-B1, OL-B2 and OL-B3 emitting light in different wavelength regions may be configured to emit white light.

Between neighboring light emitting structures OL-B1, OL-B2 and OL-B3, charge generating layers CGL1 and CGL2 may be located or disposed. The charge generating layers CGL1 and CGL2 may include a p-type or kind charge generating layer and/or an n-type or kind charge generating layer.

In at least one among the light emitting structures OL-B1, OL-B2 and OL-B3, included in the display device DD-TD of an embodiment, the amine compound of an embodiment may be included.

Referring to FIG. 9, a display device DD-b according to an embodiment may include light emitting elements ED-1, ED-2 and ED-3, formed by stacking two emission layers. Compared to the display device DD of an embodiment, shown in FIG. 2, an embodiment shown in FIG. 9 is different in that first to third light emitting elements ED-1, ED-2 and ED-3 each include two emission layers stacked in a thickness direction. In the first to third light emitting elements ED-1, ED-2 and ED-3, two emission layers may be configured to emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and/or between the first blue emission layer EML-B1 and the second blue emission layer EML-B2, an emission auxiliary part OG may be located or disposed.

The emission auxiliary part OG may include a single layer or a multilayer. The emission auxiliary part OG may include a charge generating layer. More particularly, the emission auxiliary part OG may include an electron transport region, a charge generating layer, and a hole transport region stacked in order. The emission auxiliary part OG may be provided as a common layer in all of the first to third light emitting elements ED-1, ED-2 and ED-3. However, an embodiment of the present disclosure is not limited thereto, and the emission auxiliary part OG may be patterned and provided in an opening part OH defined in a pixel definition layer PDL.

The first red emission layer EML-R1, the first green emission layer EML-G1 and/or the first blue emission layer EML-B1 may be located or disposed between the electron transport region ETR and the emission auxiliary part OG. The second red emission layer EML-R2, the second green emission layer EML-G2 and the second blue emission layer EML-B2 may be located or disposed between the emission auxiliary part OG and the hole transport region HTR.

For example, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order. The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

In some embodiments, an optical auxiliary layer PL may be located or disposed on a display element layer DP-ED. The optical auxiliary layer PL may include a polarization layer. The optical auxiliary layer PL may be located or disposed on a display panel DP and may control reflected light at the display panel DP by external light. Different from the drawings, the optical auxiliary layer PL may not be provided from the display device according to an embodiment.

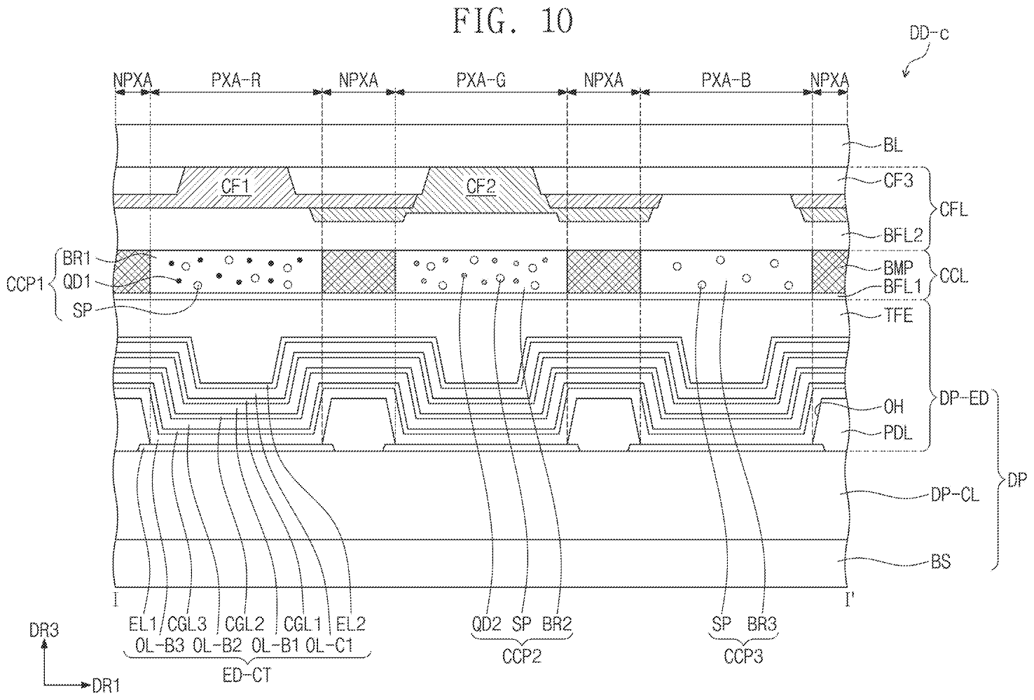

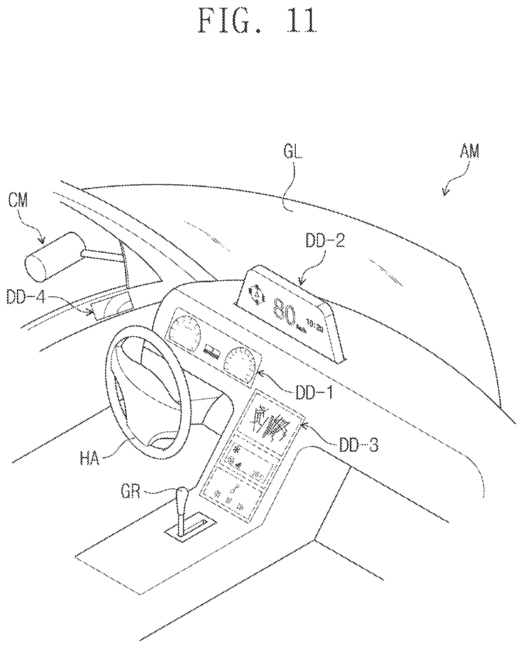

Referring to FIG. 10, a display device DD-c is configured to include four light emitting structures OL-B1, OL-B2, OL-B3 and OL-C1, different from FIG. 8 and FIG. 9. A light emitting element ED-CT may include oppositely disposed first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be located or disposed. Among the four light emitting structures, the first to third light emitting structures OL-B1, OL-B2 and OL-B3 may be configured to emit blue light, and the fourth light emitting structure OL-C1 may be configured to emit green light. However, an embodiment of the present disclosure is not limited thereto, and the first to fourth light emitting structures OL-B1, OL-B2, OL-B3 and OL-C1 may be configured to emit different wavelengths of light.

Charge generating layers CGL1, CGL2 and CGL3 located or disposed among neighboring light emitting structures OL-B1, OL-B2, OL-B3 and OL-C1 may include a p-type or kind charge generating layer and/or an n-type or kind charge generating layer.

In at least one among the light emitting structures OL-B1, OL-B2, OL-B3 and OL-C1, included in the display device DD-c of an embodiment, the amine compound of an embodiment may be included.

The light emitting element ED according to an embodiment of the present disclosure may include the amine compound of an embodiment in at least one functional layer located or disposed between the first electrode EL1 and the second electrode EL2 to show enhanced or improved emission efficiency (e.g., luminance emission efficiency) and/or enhanced or improved lifetime characteristics. The light emitting element ED according to an embodiment may include the amine compound of an embodiment in at least one among a hole transport region HTR, an emission layer EML, and an electron transport region ETR, disposed between the first electrode EL1 and the second electrode EL2, or in a capping layer CPL. For example, the amine compound according to an embodiment may be included in the hole transport region HTR of the light emitting element ED of an embodiment, and the light emitting element of an embodiment may show high efficiency (e.g., luminance emission efficiency) and/or long lifetime characteristics.

The amine compound of an embodiment including a core nitrogen atom connected to first, second, and third substituents may enhance or improve the stability of a material (e.g., a material utilized in a hole transport region or layer) and improve the hole transport properties thereof. Accordingly, the lifetime and efficiency (e.g., luminance emission efficiency) of the light emitting element including the amine compound of an embodiment may be enhanced or improved. In some embodiments, the light emitting element of an embodiment that includes the amine compound according to an embodiment in a hole transport layer may have (i.e., exhibit or show) enhanced or improved efficiency (e.g., luminance emission efficiency) and/or lifetime characteristics.

FIG. 11 is a diagram showing a vehicle (e.g., an automobile) AM in which first to fourth display apparatuses DD-1, DD-2, DD-3 and DD-4 are located or disposed. At least one among the first to fourth display apparatuses DD-1, DD-2, DD-3 and DD-4 may include the same configurations of the display apparatuses DD, DD-TD, DD-a, DD-b and DD-c of embodiments, explained referring to FIGS. 1, 2, and 7 to 10.

In FIG. 11, a vehicle is shown as an automobile AM, but this is an illustration, and the first to fourth display apparatuses DD-1, DD-2, DD-3 and DD-4 may be located or disposed on other transportation vehicles such as bicycles, motorcycles, trains, ships (e.g., ocean ships) and airplanes. In some embodiments, at least one among the first to fourth display apparatuses DD-1, DD-2, DD-3 and DD-4 including the same configurations of the display apparatuses DD, DD-TD, DD-a, DD-b and DD-c may be introduced in personal computers, laptop computers, personal digital terminals, game consoles, portable electronic devices, televisions, monitors, external billboards, and/or the like. In some embodiments, these are suggested as examples, and the display apparatus may be introduced in other electronic devices as long as not deviated from the present disclosure.

At least one among the first to fourth display apparatuses DD-1, DD-2, DD-3 and DD-4 may include the light emitting element ED of an embodiment, explained referring to FIG. 3 to FIG. 6. The light emitting element ED of an embodiment may include the amine compound of an embodiment. At least one among the first to fourth display apparatuses DD-1, DD-2, DD-3 and DD-4 may include the light emitting element ED including the amine compound of an embodiment and may exhibit or show enhanced or improved display lifetime.

Referring to FIG. 11, an automobile AM may include a steering wheel HA for the operation of the automobile AM and a gear GR. In some embodiments, the automobile AM may include a front window GL disposed to face a driver.

A first display apparatus DD-1 may be located or disposed in a first region overlapping with the steering wheel HA. For example, the first display apparatus DD-1 may be a digital cluster displaying the first information of the automobile AM. The first information may include a first graduation showing the running speed of the automobile AM, a second graduation showing the number of revolution of an engine (i.e., revolutions per minute (RPM)), and images showing a fuel state. First graduation and second graduation may be represented by digital images.

A second display apparatus DD-2 may be located or disposed in a second region facing a driver's seat and overlapping with the front window GL. The driver's seat may be a seat where the steering wheel HA is disposed. For example, the second display apparatus DD-2 may be a head up display (HUD) showing the second information of the automobile AM. The second display apparatus DD-2 may be optically clear. The second information may include digital numbers showing the running speed of the automobile AM and may further include information including the current time. Different from the drawing, the second information of the second display apparatus DD-2 may be projected and displayed on the front window GL.

A third display apparatus DD-3 may be located or disposed in a third region adjacent to the gear GR. For example, the third display apparatus DD-3 may be a center information display (CID) for an automobile, located or disposed between a driver's seat and a passenger seat and showing third information. The passenger seat may be a seat separated from the driver's seat with the gear GR therebetween. The third information may include information on road conditions (for example, navigation information), on playing music or radio, on playing a dynamic image (or image), on the temperature in the automobile AM, and/or the like.

A fourth display apparatus DD-4 may be located or disposed in a fourth region separated from the steering wheel HA and the gear GR and adjacent to the side of the automobile AM. For example, the fourth display apparatus DD-4 may be a digital wing mirror displaying fourth information. The fourth display apparatus DD-4 may display the external image of the automobile AM, taken by a camera module CM disposed at the outside of the automobile AM. The fourth information may include the external image of the automobile AM.

The described first to fourth information is for illustration, and the first to fourth display apparatuses DD-1, DD-2, DD-3 and DD-4 may further display information on the inside and outside of the automobile. The first to fourth information may include different information from each other. However, an embodiment of the present disclosure is not limited thereto, and a portion of the first to fourth information may include the same information.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting element, the display device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting element and/or the display device may be formed on one integrated circuit (IC) chip or on separate IC chips. Further, the various components of the elements and/or devices may be implemented on a flexible printed circuit film, a tape carrier package (TCP), a printed circuit board (PCB), or formed on one substrate. Further, the various components of the elements and/or devices may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when particles are spherical, “diameter” indicates an average particle diameter, and when the particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Hereinafter, referring to example embodiments and comparative example embodiments, the amine compound and the light emitting element according to one or more embodiments of the present disclosure will be described and/or explained in particular. In some embodiments, the example embodiments are illustrations to assist the understanding of the present disclosure, but the scope of the present disclosure is not limited thereto.

### EXAMPLES

### 1. Synthesis of Amine Compounds

First, the synthetic methods of the amine compounds according to embodiments will be described to illustrate the synthetic methods of Compound A185, Compound A186, Compound A899, Compound A900, Compound A924, Compound B299, and Compound B922. In some embodiments, the synthetic methods of the amine compounds disclosed hereinafter are embodiments, and the synthetic method of the amine compound according to an embodiment of the present disclosure is not limited to the embodiments. In some embodiments, in the synthesis of the amine compounds, the molecular weight of the amine compounds were measured by FAB-MS utilizing JMS-700V of JEOL Co.

### (1) Synthesis of Compound A185

Amine Compound A185 according to an embodiment may be synthesized by, for example, the reactions as described.

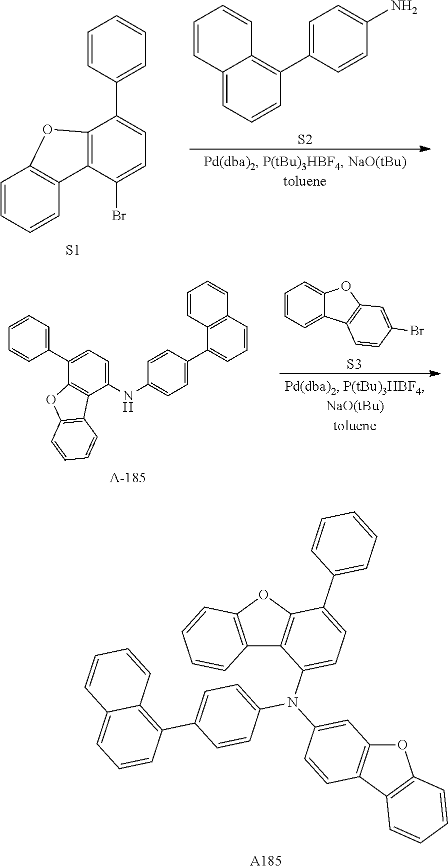

### (Synthesis of Compound A-185)

Under an Ar atmosphere, to a 1000 mL three-neck flask, Compound S1 (10.18 g, 46.41 mmol), Pd(dba)₂ (1.33 g, 0.05 equiv, 2.32 mmol), NaOtBu (4.46 g, 1 equiv, 46.41 mmol), toluene (464 mL), Compound S2 (15.48 g, 1.0 equiv, 46.41 mmol) and P(tBu)₃HBF₄ (2.69 g, 0.2 equiv, 9.28 mmol) were added one by one, followed by heating, refluxing and stirring for about 6 hours. After cooling to room temperature, water was added to the reaction mixture, and an organic layer was separately taken. Toluene was added to the aqueous layer, and an organic layer was additionally extracted. Organic layers were collected, washed with a saline solution and dried over MgSO₄. MgSO₄ was filtered, and an organic layer was concentrated. The crude product thus obtained was purified by silica gel column chromatography to obtain Compound A-185 as a white solid (18.6 g, yield 87%).

By FAB-MS measurement, mass number of m/z=461 was observed as a molecular ion peak, and Compound A-185 was identified.

### (Synthesis of Compound A185)

Under an Ar atmosphere, to a 500 mL three-neck flask, Compound A-185 (10.0 g, 21.67 mmol), Pd(dba)₂ (0.62 g, 0.05 equiv, 1.08 mmol), NaOtBu (2.08 g, 1 equiv, 21.67 mmol), toluene (216 mL), Compound S3 (6.98 g, 1.0 equiv, 21.67 mmol) and P(tBu)₃HBF₄ (1.261 g, 0.2 equiv, 4.33 mmol) were added one by one, followed by heating, refluxing and stirring for about 6 hours. After cooling to room temperature, water was added to the reaction mixture, and an organic layer was separately taken. Toluene was added to the aqueous layer, and an organic layer was additionally extracted. Organic layers were collected, washed with a saline solution and dried over MgSO₄. MgSO₄ was filtered, and an organic layer was concentrated. The crude product thus obtained was purified by silica gel column chromatography to obtain Compound A185 as a white solid (9.5 g, yield 70%).

By FAB-MS measurement, mass number of m/z=627 was observed as a molecular ion peak, and Compound A185 was identified.

### (2) Synthesis of Compound A186

Amine Compound A186 according to an embodiment may be synthesized by, for example, the reactions as described.

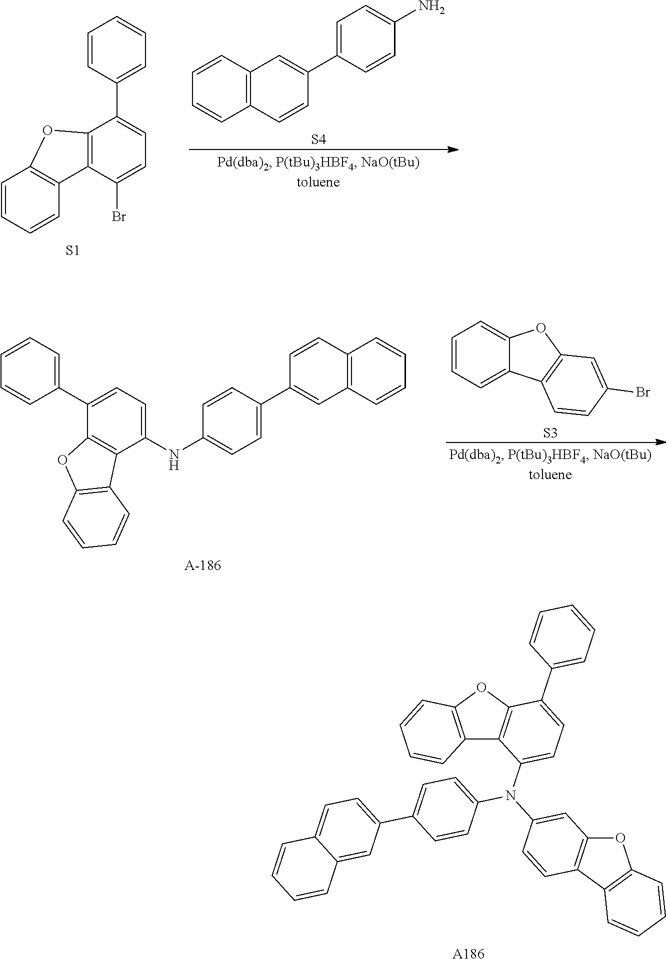

Compound A186 was synthesized by the same method as the synthetic method of Compound A185 except for utilizing Compound S4 instead of Compound S2.

By FAB-MS measurement, mass number of m/z=627 was observed as a molecular ion peak, and Compound A186 was identified.

### (3) Synthesis of Compound A899

Amine Compound A899 according to an embodiment may be synthesized by, for example, the reactions as 150 escribed.

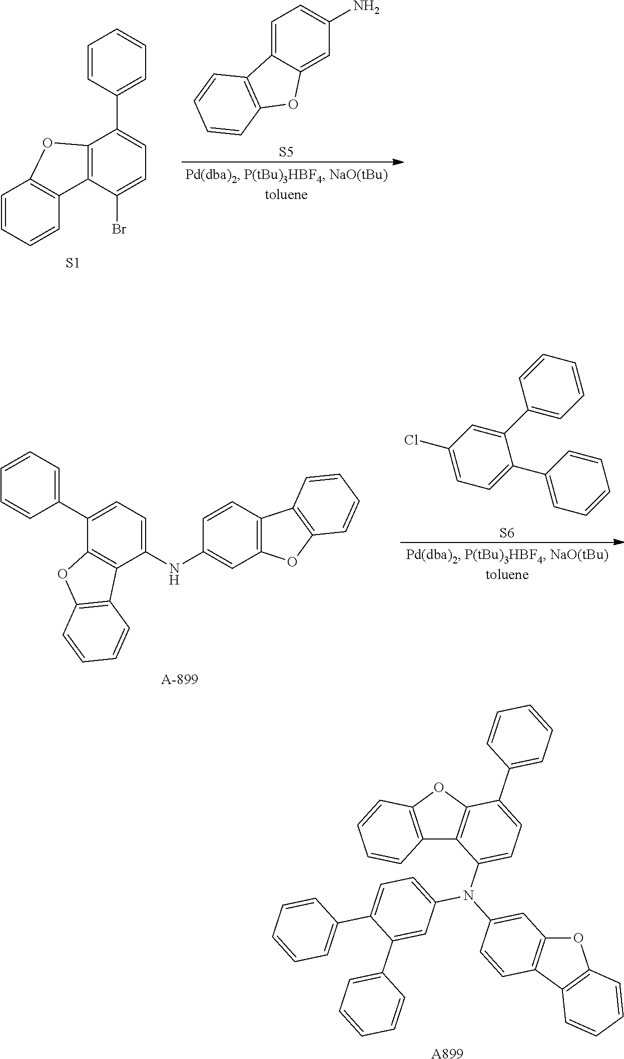

Compound A899 was synthesized by the same method as the synthetic method of Compound A185 except for utilizing Compound S5 instead of Compound S2 and utilizing Compound S6 instead of Compound S3.

By FAB-MS measurement, mass number of m/z=653 was observed as a molecular ion peak, and Compound A899 was identified.

### (4) Synthesis of Compound A900

Amine Compound A900 according to an embodiment may be synthesized by, for example, the reactions as described.

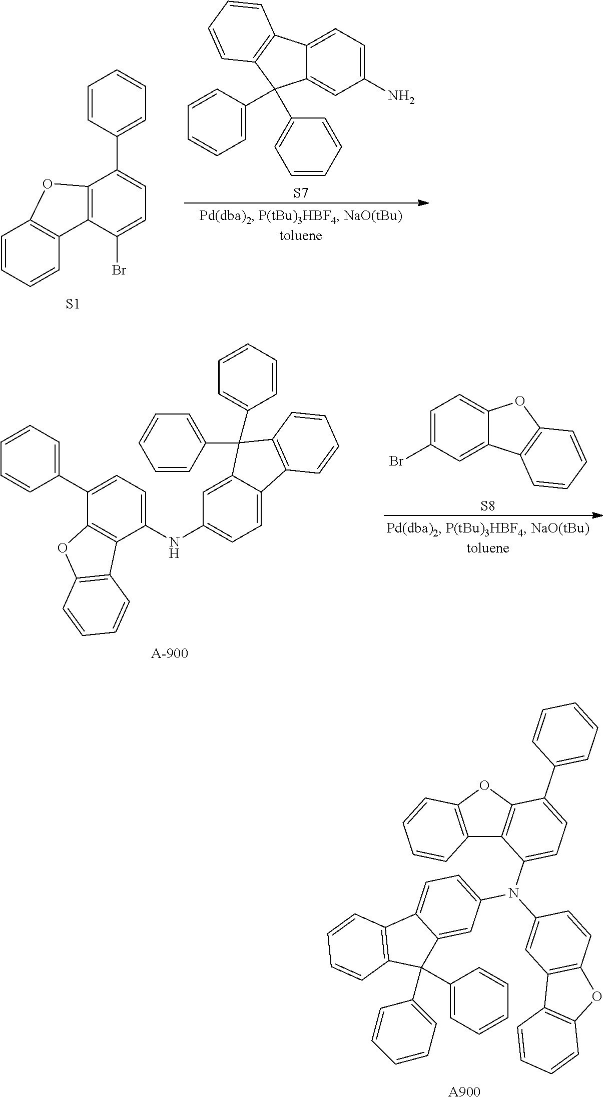

Compound A900 was synthesized by the same method as the synthetic method of Compound A185 except for utilizing Compound S7 instead of Compound S2, and utilizing Compound S8 instead of Compound S3.

By FAB-MS measurement, mass number of m/z=741 was observed as a molecular ion peak, and Compound A900 was identified.

### (5) Synthesis of Compound A924

Amine Compound A924 according to an embodiment may be synthesized by, for example, the reactions as described.

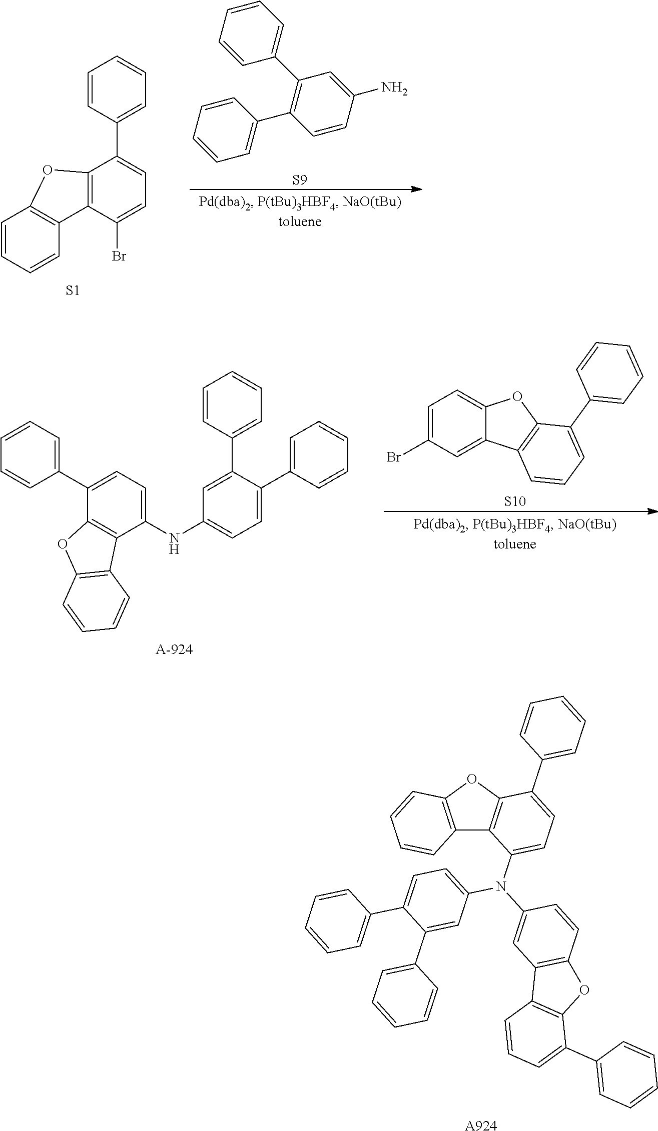

Compound A924 was synthesized by the same method as the synthetic method of Compound A185 except for utilizing Compound S9 instead of Compound S2, and utilizing Compound S10 instead of Compound S3.

By FAB-MS measurement, mass number of m/z=729 was observed as a molecular ion peak, and Compound A924 was identified.

### (6) Synthesis of Compound B299

Amine Compound B299 according to an embodiment may be synthesized by, for example, the reactions as described.

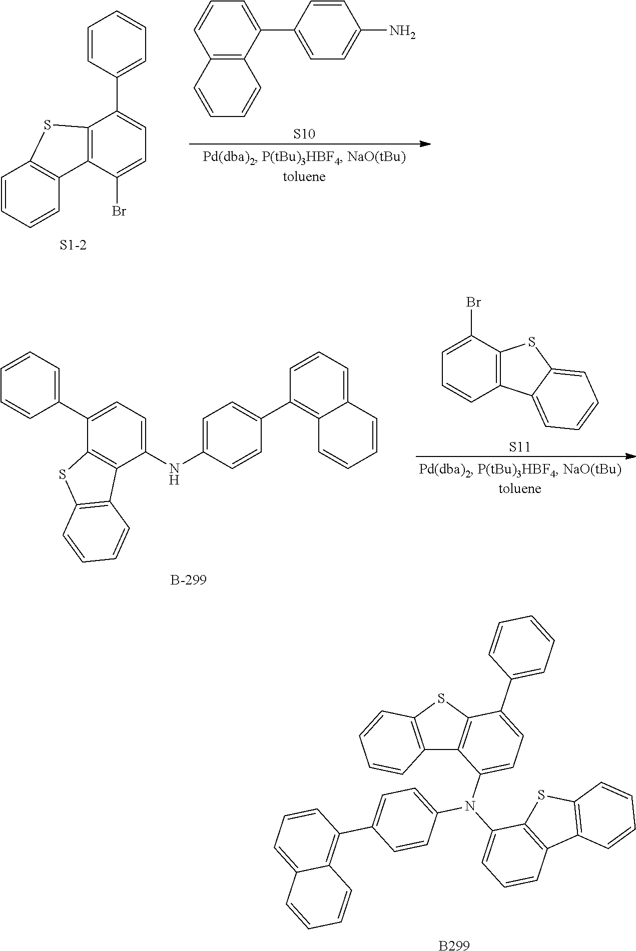

Compound B299 was synthesized by the same method as the synthetic method of Compound A185 except for utilizing Compound S1-2 instead of Compound S1, utilizing Compound S10 instead of Compound S2, and utilizing Compound S11 instead of Compound S3.

By FAB-MS measurement, mass number of m/z=659 was observed as a molecular ion peak, and Compound B299 was identified.

### (7) Synthesis of Compound B922

Amine Compound B922 according to an embodiment may be synthesized by, for example, the reactions as described.

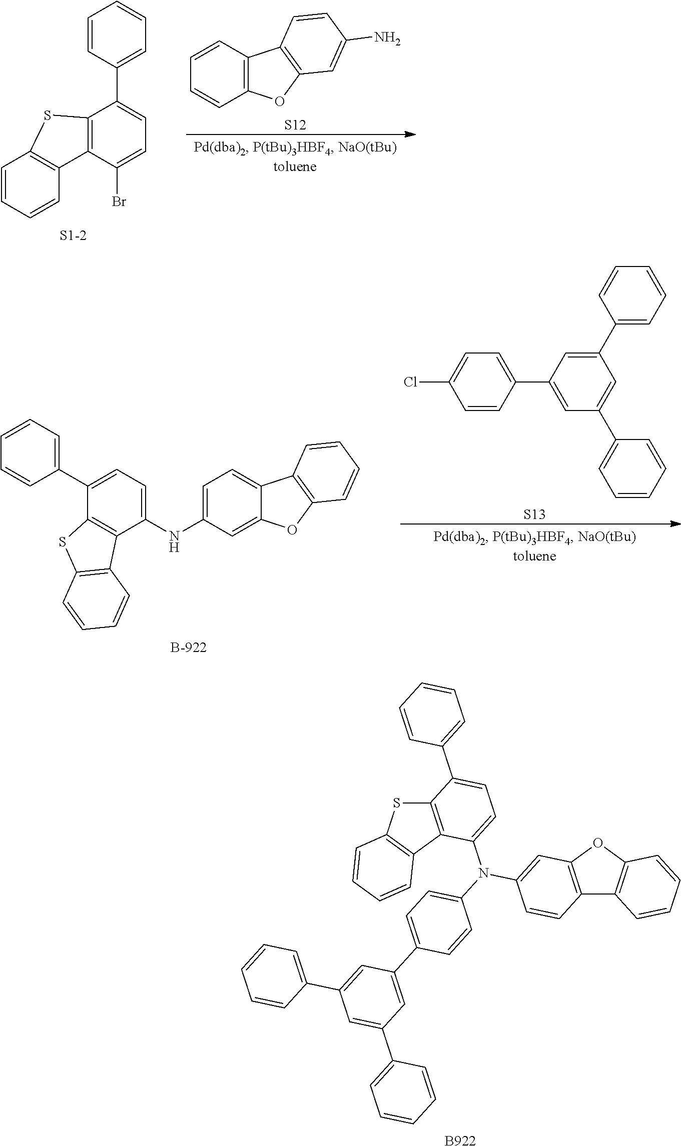

Compound B922 was synthesized by the same method as the synthetic method of Compound A185 except for utilizing Compound S1-2 instead of Compound S1, utilizing Compound S12 instead of Compound S2, and utilizing Compound S13 instead of Compound S3.

By FAB-MS measurement, mass number of m/z=745 was observed as a molecular ion peak, and Compound B922 was identified.

### 2. Manufacture and Evaluation of Light Emitting Elements

A light emitting element of an embodiment, including the amine compound of an embodiment in a hole transport layer were manufactured. Light emitting elements of Example 1 to Example 7 were manufactured utilizing the amine compounds of Compound A185, Compound A186, Compound A899, Compound A900, Compound A924, Compound B299, and Compound B922 as a material in the hole transport layer. Comparative Example 1 to Comparative Example 7 correspond to light emitting elements manufactured utilizing Comparative Compounds R1 to R7 as a material in the hole transport layer.

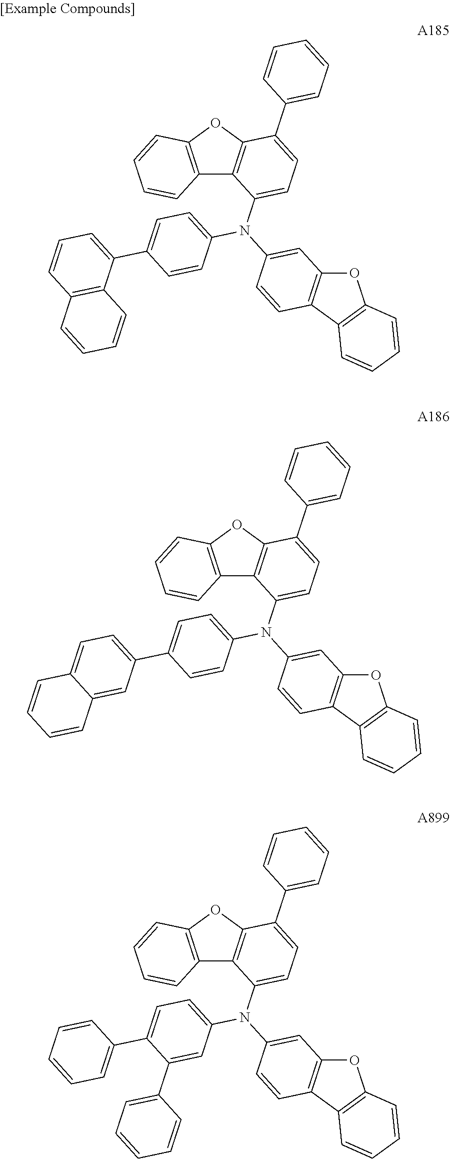 [SMILES: c1ccc(-c2ccc(N(c3ccc(-c4cccc5ccccc45)cc3)c3ccc4c(c3)oc3ccccc34)c3c2oc2ccccc23)cc1 ; c1ccc(-c2ccc(N(c3ccc(-c4ccc5ccccc5c4)cc3)c3ccc4c(c3)oc3ccccc34)c3c2oc2ccccc23)cc1 ; c1ccc(-c2ccc(N(c3ccc4c(c3)oc3ccccc34)c3ccc(-c4ccccc4)c4oc5ccccc5c34)cc2-c2ccccc2)cc1]

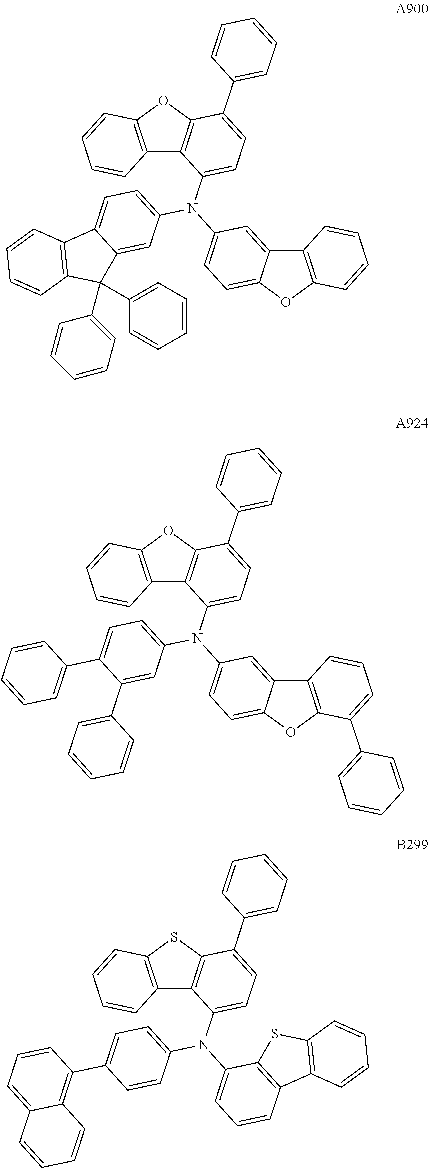 [SMILES: c1ccc(-c2ccc(N(c3ccc4c(c3)C(c3ccccc3)(c3ccccc3)c3ccccc3-4)c3ccc4oc5ccccc5c4c3)c3c2oc2ccccc23)cc1 ; c1ccc(-c2ccc(N(c3ccc4oc5c(-c6ccccc6)cccc5c4c3)c3ccc(-c4ccccc4)c4oc5ccccc5c34)cc2-c2ccccc2)cc1 ; c1ccc(-c2ccc(N(c3ccc(-c4cccc5ccccc45)cc3)c3cccc4c3sc3ccccc34)c3c2sc2ccccc23)cc1]

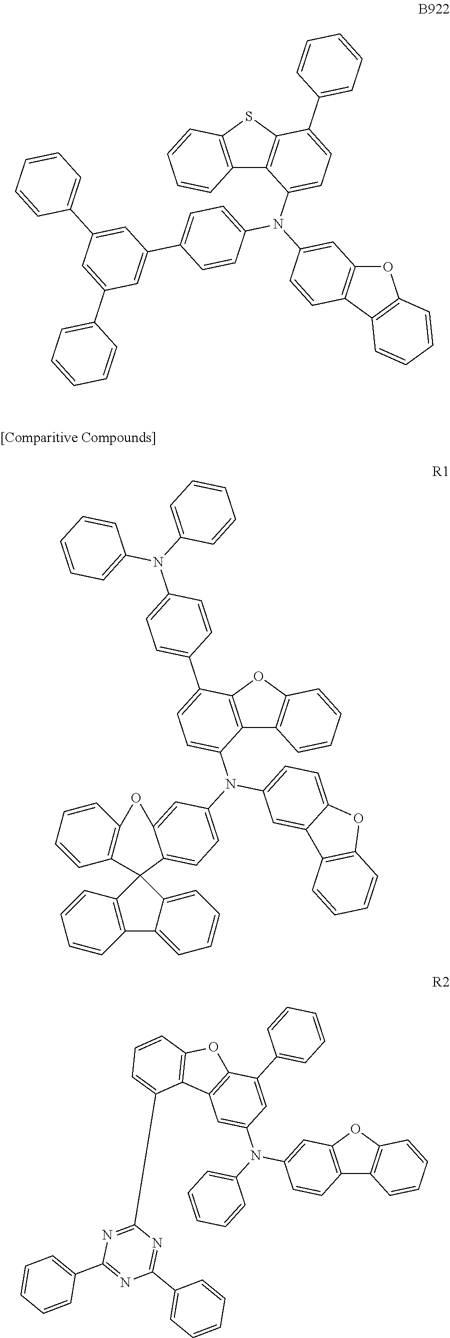 [SMILES: c1ccc(-c2cc(-c3ccccc3)cc(-c3ccc(N(c4ccc5c(c4)oc4ccccc45)c4ccc(-c5ccccc5)c5sc6ccccc6c45)cc3)c2)cc1 ; c1ccc(N(c2ccccc2)c2ccc(-c3ccc(N(c4ccc5c(c4)Oc4ccccc4C54c5ccccc5-c5ccccc54)c4ccc5oc6ccccc6c5c4)c4c3oc3ccccc34)cc2)cc1 ; c1ccc(-c2nc(-c3ccccc3)nc(-c3cccc4oc5c(-c6ccccc6)cc(N(c6ccccc6)c6ccc7c(c6)oc6ccccc67)cc5c34)n2)cc1]

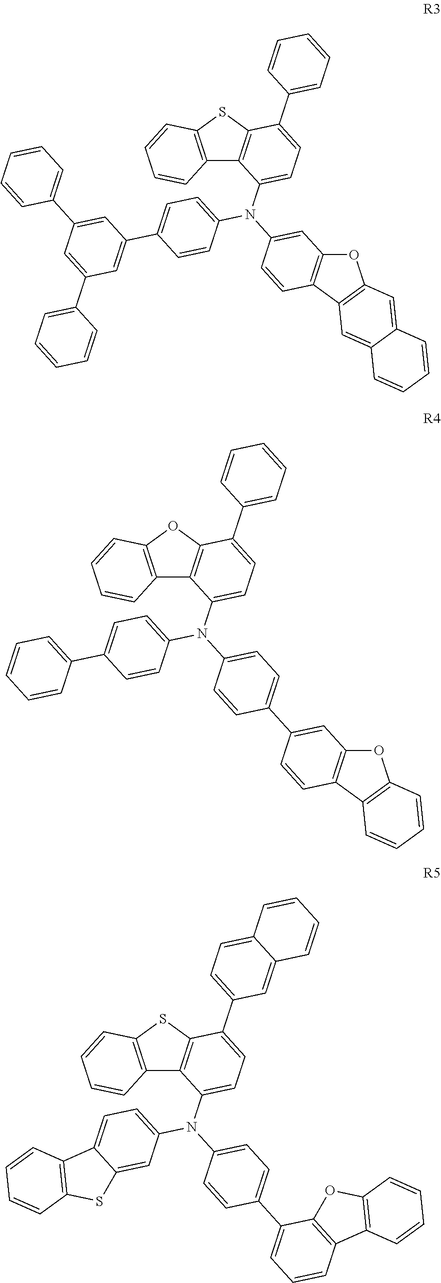 [SMILES: c1ccc(-c2cc(-c3ccccc3)cc(-c3ccc(N(c4ccc5c(c4)oc4cc6ccccc6cc45)c4ccc(-c5ccccc5)c5sc6ccccc6c45)cc3)c2)cc1 ; c1ccc(-c2ccc(N(c3ccc(-c4ccc5c(c4)oc4ccccc45)cc3)c3ccc(-c4ccccc4)c4oc5ccccc5c34)cc2)cc1 ; c1ccc2cc(-c3ccc(N(c4ccc(-c5cccc6c5oc5ccccc56)cc4)c4ccc5c(c4)sc4ccccc45)c4c3sc3ccccc34)ccc2c1]

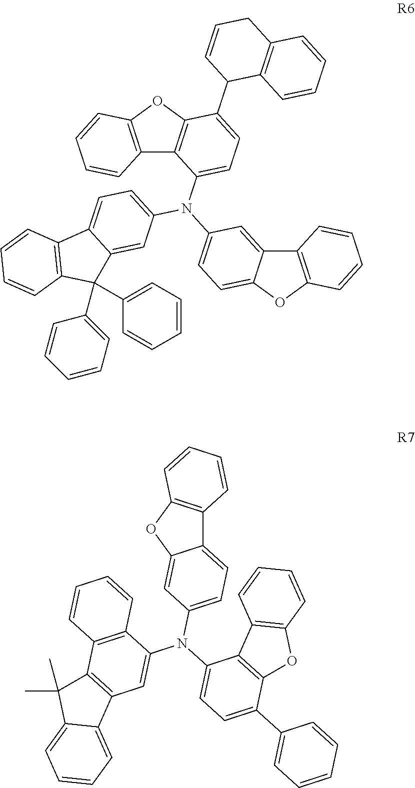 [SMILES: C1=CC(c2ccc(N(c3ccc4c(c3)C(c3ccccc3)(c3ccccc3)c3ccccc3-4)c3ccc4oc5ccccc5c4c3)c3c2oc2ccccc23)c2ccccc2C1 ; CC1(C)c2ccccc2-c2cc(N(c3ccc4c(c3)oc3ccccc34)c3ccc(-c4ccccc4)c4oc5ccccc5c34)c3ccccc3c21]

### 3. Manufacture of Light Emitting Elements

An ITO glass substrate with about 15 ohms per square centimeter (Ω/cm²) (about 1500 angstrom (Å)) obtained from the Corning Co. was cut into a size of 50 mm×50 mm×0.7 mm, washed with isopropyl alcohol and ultrapure water, cleansed utilizing ultrasonic waves for about 5 minutes, exposed to UV for about 30 minutes and treated with ozone. Then, 4,4′,4″-tris{N,-(2-naphthyl)-N-phenylamino}-triphenylamine (2-TNATA) was vacuum deposited to a thickness of about 600 Å to form a hole injection layer. After that, the Example Compound or Comparative Compound was vacuum deposited to a thickness of about 300 Å to form a hole transport layer.

On the hole transport layer, a blue fluorescence host of 9,10-di(naphthalen-2-yl)anthracene (ADN) and a fluorescence dopant of 2,5,8,11-tetra-t-butylperylene (TBP) were co-deposited in a ratio (e.g., amount) of about 97:3 to form an emission layer with a thickness of about 250 Å.

On the emission layer, an electron transport layer was formed to a thickness of about 250 Å utilizing tris(8-hydroxyquinolino)aluminum (Alq₃), and then, an electron injection layer was formed to a thickness of about 10 Å by depositing LiF. On the electron injection layer, a second electrode was formed to a thickness of about 1000 Å utilizing aluminum (Al).

In some embodiments, the compounds of the functional layers utilized for the manufacture of the light emitting elements are as follows.

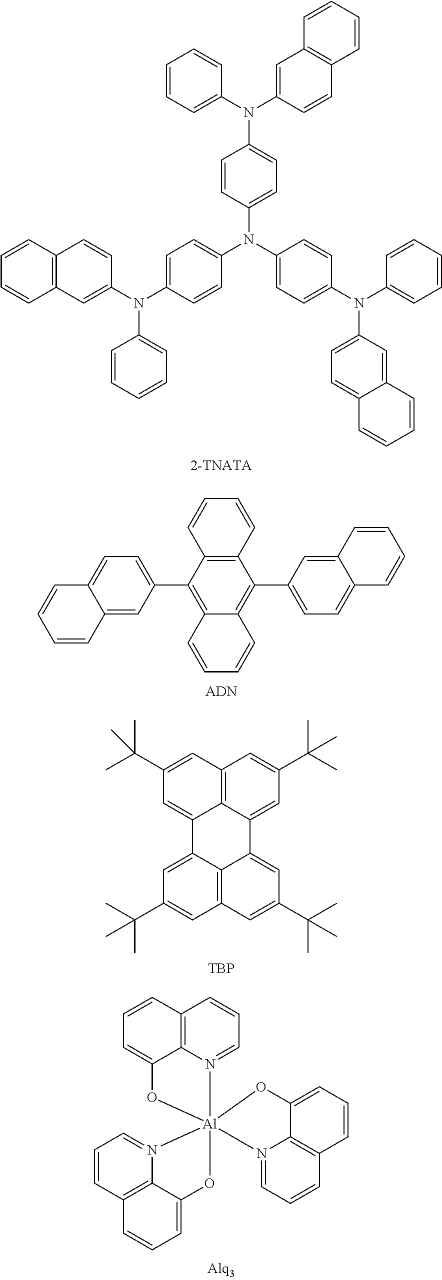

### 4. Evaluation of Light Emitting Elements

Table 1 shows evaluation results of the light emitting elements of Examples 1 to 7, and Comparative Examples 1 to 7. In Table 1, the luminance emission efficiency (i.e., Efficiency) is reported as candela per ampere, and half-life (i.e., Lifetime LT50) is reported in hours.

In the evaluation results of the properties of the Examples and Comparative Examples, shown in Table 1, the maximum luminance emission efficiency values were measured at a current density of about 10 milliamps per square centimeter (mA/cm²). The luminance half-life values were measured from an initial luminance of about 1000 candela per square meter (cd/m²) to a point where the luminance was reduced to half of the initial value at a current density of about 10 mA/cm². The evaluation of the current density and luminance emission efficiency of the light emitting elements was conducted utilizing a Source Meter of 2400 series, which is a product of Keithley Instruments Co., a luminance and color meter of CS-200, which is a product of Konica Minolta Co., Ltd., and PC Program LabVIEW 8.2 for measurement, which is a product of National Instruments Co., Ltd., in a dark room.

TABLE 1

| Light emitting | Hole transport | Voltage | Efficiency | Lifetime |
|---|---|---|---|---|
| element | layer | (V) | (cd/A) | LT50 (h) |
| | Example | | | |
| Example ID | compound ID | | | |
| 1 | A185 | 5.3 | 9.2 | 2460 |
| 2 | A186 | 5.4 | 8.8 | 2500 |
| 3 | A899 | 5.2 | 8.9 | 2450 |
| 4 | A900 | 5.3 | 8.8 | 2430 |
| 5 | A924 | 5.6 | 8.5 | 2380 |
| 6 | B299 | 5.2 | 8.7 | 2350 |
| 7 | B922 | 5.2 | 8.6 | 2250 |
| Comparative | Comparative | | | |
| Example ID | compound ID | | | |
| 1 | 1 | 6.0 | 6.2 | 1600 |
| 2 | 2 | 6.5 | 6.0 | 1300 |
| 3 | 3 | 5.9 | 7.4 | 1700 |
| 4 | 4 | 6.1 | 7.5 | 1900 |
| 5 | 5 | 6.0 | 7.2 | 1800 |
| 6 | 6 | 5.8 | 7.5 | 1100 |
| 7 | 7 | 5.9 | 7.5 | 1400 |

Referring to the results of Table 1, the Examples of the light emitting elements utilizing the amine compounds according to embodiments of the present disclosure, as a material in the hole transport layer, showed lower driving voltages, enhanced or higher luminance emission efficiency (i.e., Efficiency), and enhanced or longer element lifetime (e.g., Lifetime LT50 or half-life) when compared to the Comparative Examples. The Example Compounds are tertiary amine compounds (each) including a first substituent, a second substituent and a third substituent, connected to or with a core nitrogen atom and they include the first substituent having a dibenzoheterole structure connected to or with the core nitrogen atom at a carbon position 1. Accordingly, charge density may be enhanced or increased, hole transport capacity may be enhanced or improved, and efficiency (e.g., luminance emission efficiency) may be enhanced or improved. In some embodiments, because an aryl group is substituted (e.g., essentially substituted) at any one carbon position among carbon atoms at positions 2 to 4 in the first substituent, several beneficial effects may occur. For example, the amount of conjugation in the structure of the amine compound may be enhanced or increased, the stability of the molecular structure of the amine compound may be enhanced or improved, and/or, when utilized in a light emitting element, the element lifetime may be enhanced or improved. In some embodiments, because an aryl group is substituted at any one carbon position among carbon atoms at positions 2 to 4 in the first substituent, intermolecular stacking may be enhanced or improved (e.g., may occur easily), intermolecular distance may be reduced, and/or hole transport capacity may be enhanced or improved. Because the amine compound of an embodiment additionally includes a second substituent having a dibenzoheterole structure, hole transport capacity may be additionally enhanced or improved, and/or the stability of a radical cation state may be enhanced or improved. Accordingly, it could be expected that light emitting elements according to the Examples, that include such a compound of an embodiment as a hole transport material, may exhibit (e.g., show) high luminance emission efficiency and long element lifetime.

Comparative Example 1 showed a high driving voltage and degraded element lifetime and efficiency when compared to Examples 1 to 7. In the case of utilizing or applying Comparative Compound R1 in or to a light emitting element, it is thought that the luminance emission efficiency and lifetime were degraded. In some embodiments, Comparative Compound R1 has a benzidine structure in a molecular structure (a structure in which two nitrogen atoms at positions p and p′ of biphenyl are facing). Accordingly, it is thought that hole transport capacity was high, but increased reactivity with nearby (e.g., surrounding) molecules may have caused the element to be deteriorated during driving to degrade the luminance emission efficiency and lifetime.

Comparative Example 2 showed a high driving voltage and degraded element lifetime and efficiency when compared to Examples 1 to 7. In the case of Comparative Compound R2, different from the Example Compounds, a nitrogen-containing six-member heterocycle is substituted at a dibenzoheterole moiety. Accordingly, it is thought that electron transport capacity may have increased, and the charge balance of an element became poor, such that luminance emission efficiency and lifetime were degraded.

Comparative Example 3 showed a high driving voltage and degraded element lifetime and efficiency when compared to Examples 1 to 7. Comparative Compound R3 has a benzonaphthofuran moiety in a molecular structure, and the fused ring skeleton of the corresponding structure has a relatively high deposition temperature due to planarity. Accordingly, it is thought that the stability of a compound may have deteriorated, e.g., causing decomposition during deposition, and when utilized in, or applied to, a light emitting element, luminance emission efficiency and lifetime were degraded.

Comparative Examples 4 and 5 showed a high driving voltage and degraded element lifetime and efficiency when compared to Examples 1 to 7. In the cases of Comparative Compounds R4 and R₅, different from the Example Compounds, a dibenzofuran moiety corresponding to the second substituent does not make a direct linkage but is combined via a phenylene linker. Accordingly, effects of a HOMO level and charge mobility may have degraded, and when utilized in, or applied to, a light emitting element, it is thought that luminance emission efficiency and lifetime were degraded.

Comparative Example 6 showed a high driving voltage and degraded element lifetime and efficiency when compared to Examples 1 to 7. Comparative Compound R6 has a structure in which a substituent corresponding to Ar₁ of the first substituent is 1,4-hydronaphthyl. Accordingly, it is thought that a partially reduced naphthalene structure is configured to easily undergo oxidation reactions and would be unstable, and reduce element lifetime thereby.

Comparative Example 7 showed a high driving voltage and degraded element lifetime and efficiency when compared to Examples 1 to 7. Due to the planarity of a benzofluorene moiety included in Comparative Compound R7, the deposition temperature of a compound is relatively high. Accordingly, it is thought that the stability of the compound may have been degraded, e.g., due to decomposition during deposition, and when utilized in, or applied to, a light emitting element, luminance emission efficiency and lifetime were degraded.

In the case of applying the Comparative Compounds in a light emitting element, it could be confirmed that luminance emission efficiency (e.g., Efficiency) was degraded, and lifetime (e.g., Lifetime LT50 or half-life) was reduced when compared to the Example Compounds. For example, referring to Table 1, in the case of the light emitting element utilizing the amine compound according to an embodiment of the present disclosure, it could be confirmed that improvements to the light emitting element properties including luminance emission efficiency and element lifetime were achieved when compared to the Comparative Examples.

The light emitting element of an embodiment includes the amine compound of an embodiment and may show high efficiency (e.g., luminance emission efficiency) and long lifetime characteristics.

When the amine compound of an embodiment is utilized in a light emitting element, high efficiency (e.g., luminance emission efficiency) and long lifetime characteristics may be shown.

Although the embodiments of the present disclosure have been described, it will be understood by those skilled in the art that the present disclosure should not be limited to these embodiments, but one or more suitable changes and modifications can be made by one ordinary skilled in the art within the spirit and scope of the present disclosure as set forth in the following claims and equivalents thereof.

Accordingly, the technical scope of the present disclosure is not to be limited to the contents set forth in the detailed description of the specification, but is intended to be defined by the appended claims.

What is claimed is:

1. A light emitting element, comprising:
a first electrode;
a second electrode on the first electrode; and
at least one functional layer between the first electrode and the second electrode, and comprising an amine compound represented by Formula 1: 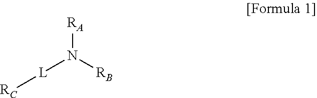
in Formula 1,
R_{A} is represented by Formula 2,
R_{B} is represented by Formula 3,
R_{C} is a substituted or unsubstituted aryl group of 6 to 30 ring-forming carbon atoms, or a substituted or unsubstituted heteroaryl group of 2 to 30 ring-forming carbon atoms,
L is a direct linkage, or a substituted or unsubstituted arylene group of 6 to 30 ring-forming carbon atoms,
R_{C} is not a substituted or unsubstituted benzonaphthofuran group, a substituted or unsubstituted benzonaphthothiophene group, or a substituted or unsubstituted benzofluorene group,
when R_{C} is a substituted or unsubstituted dibenzofuran group, or a substituted or unsubstituted dibenzothiophene group, L is a direct linkage, and
when R_{C} is a substituted or unsubstituted carbazole group, L is a direct linkage, or a substituted or unsubstituted phenylene group: 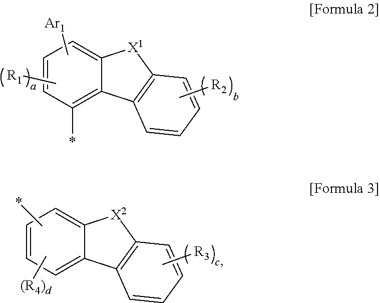
and
in Formula 2 and Formula 3,
Ar₁ is a substituted or unsubstituted aryl group of 6 to 30 ring-forming carbon atoms,
X₁ and X₂ are each independently O or S,
R₁ to R₄ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
wherein,
R₁ to R₄ are not each a substituted or unsubstituted nitrogen-containing six-member heterocycle,
Ar₁ is not a substituted or unsubstituted dihydronaphthyl group, or a substituted or unsubstituted p-aminophenyl group,
R₃ is not a substituted or unsubstituted fluorene group,
a is an integer of 0 to 2,
d is an integer of 0 to 3,
b and c are each independently an integer of 0 to 4, and
in Formula 2 when X₁ is O, Formula 3 is not represented by Formula 3-a:

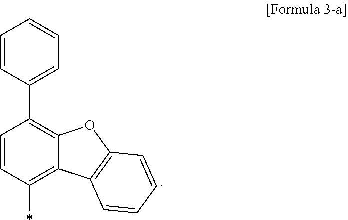 [SMILES: *c1ccc(-c2ccccc2)c2oc3ccccc3c12]

2. The light emitting element of claim 1, wherein the at least one functional layer comprises an emission layer, a hole transport region between the first electrode and the emission layer, and an electron transport region between the emission layer and the second electrode, and
wherein
the hole transport region comprises the amine compound represented by Formula 1.

3. The light emitting element of claim 2, wherein the hole transport region comprises a hole injection layer on the first electrode, and a hole transport layer on the hole injection layer, and
wherein,
the hole transport layer comprises the amine compound represented by Formula 1.

4. The light emitting element of claim 2, wherein the hole transport region comprises multiple layers, and wherein a layer of the multiple layers is adjacent to the emission layer and comprises the amine compound represented by Formula 1.

5. The light emitting element of claim 1, wherein the amine compound represented by Formula 1 is a monoamine compound.

6. The light emitting element of claim 1, wherein the amine compound represented by Formula 1 is represented by Formula 1-1: 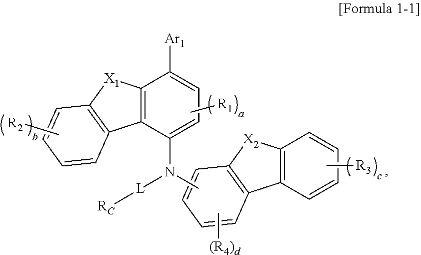
and
in Formula 1-1,
R_{C}, L, Ar₁, X₁, X₂, R₁ to R₄, and a to d are as defined in Formula 1, Formula 2 and Formula 3.

7. The light emitting element of claim 1, wherein the amine compound represented by Formula 1 is represented by Formula 1-2-1 or Formula 1-2-2: 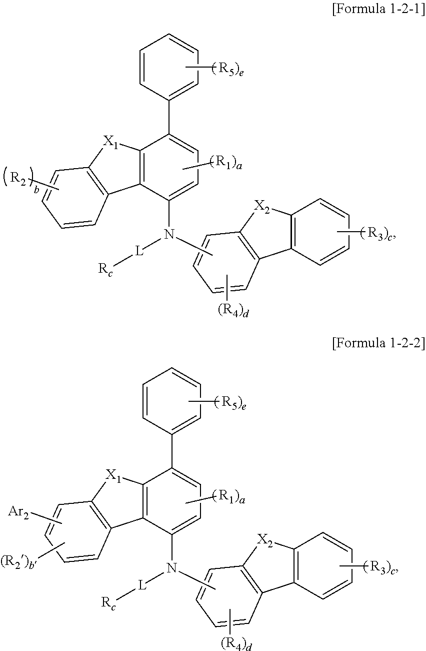
and
in Formula 1-2-1 and Formula 1-2-2,
R₂′ and R₅ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Ar₂ is a substituted or unsubstituted aryl group of 6 to 30 ring-forming carbon atoms, or a substituted or unsubstituted heteroaryl group of 2 to 30 ring-forming carbon atoms,
b′ is an integer of 0 to 3,
e is an integer of 0 to 5, and
R_{C}, L, X₁, X₂, R₁ to R₄, and a to d are as defined in Formula 1, Formula 2, and Formula 3.

8. The light emitting element of claim 1, wherein the substituent represented by Formula R_{C} is represented by one of Formula A-1 to Formula A-8: 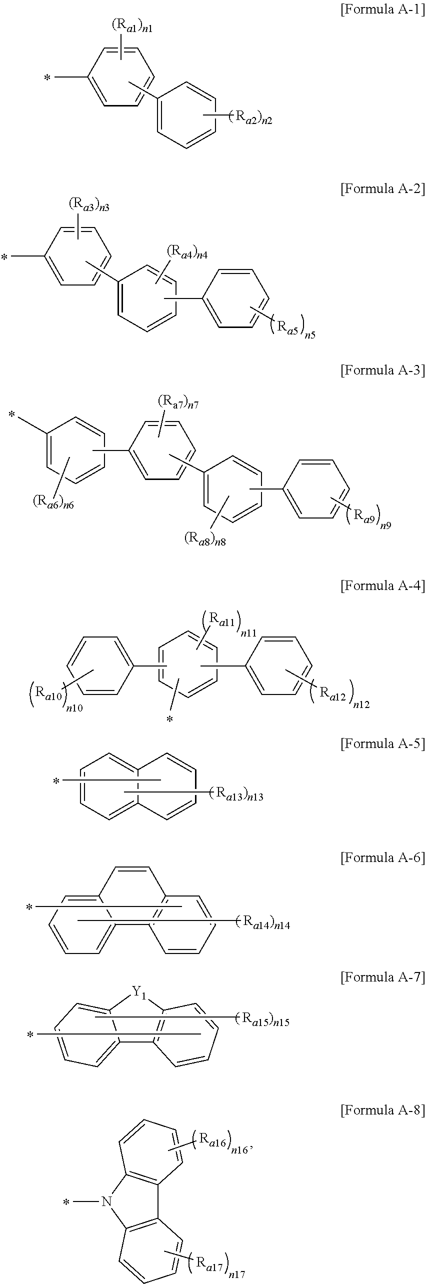
and
in Formula A-1 to Formula A-8,
Y₁ is O, S, CRₐ₁₈Rₐ₁₉, or NRₐ₂₀,
Rₐ₁ to Rₐ₁₉ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Rₐ₂₀ is a substituted or unsubstituted aryl group of 6 to 30 ring-forming carbon atoms, or a substituted or unsubstituted heteroaryl group of 2 to 30 ring-forming carbon atoms,
n1, n3, n4, and n6 to n8 are each independently an integer of 0 to 4,
n2, n5, n9, n10, and n12 are each independently an integer of 0 to 5,
n11 is an integer of 0 to 3,
n13 and n15 are each independently an integer of 0 to 7,
n14 is an integer of 0 to 9,
n16 and n17 are each independently an integer of 0 to 4, and
-* is a position connected with L of Formula 1.

9. The light emitting element of claim 1, wherein the amine compound represented by Formula 1 is represented by one of Formula 1-4-1 to Formula 1-4-3: 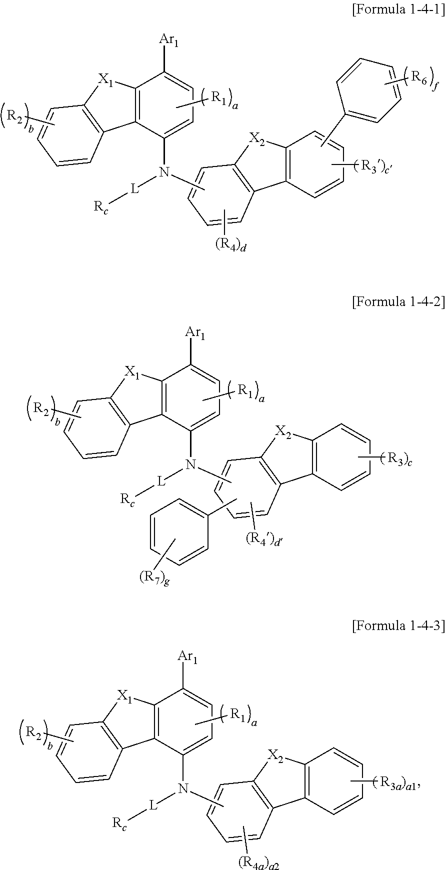
and
in Formula 1-4-1 to Formula 1-4-3,
R₃′, R₄′, R₆ and R₇ are each independently a hydrogen atom, a deuterium atom a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
R₃ₐ and R₄ₐ are each independently a hydrogen atom, or a deuterium atom,
f and g are each independently an integer of 0 to 5,
c′ and a2 are each independently an integer of 0 to 3,
d′ is an integer of 0 to 2,
a1 is an integer of 0 to 4, and
R_{C}, L, Ar₁, X₁, X₂, R₁ to R₄, and a to d are as defined in Formula 1, Formula 2, and Formula 3.

10. The light emitting element of claim 1, wherein the amine compound represented by Formula 1 is represented by one of Formula 1-5-1 or Formula 1-5-2: 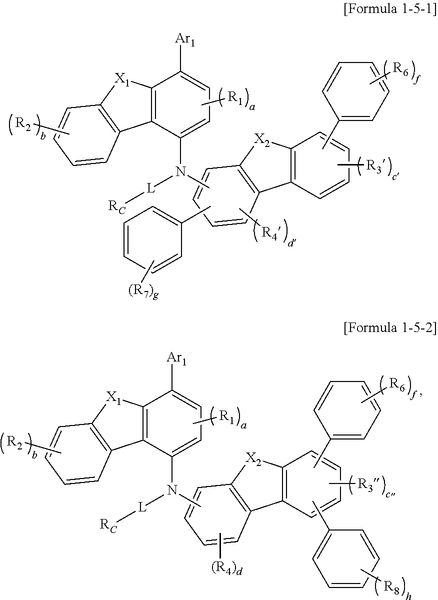
and
in Formula 1-5-1 and Formula 1-5-2,
R₃′, R₃″, R₄′, and R₆ to R₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
f to h are each independently an integer of 0 to 5,
c′ is an integer of 0 to 3,
c″ and d′ are each independently an integer of 0 to 2, and
R_{C}, L, Ar₁, X₁, X₂, R₁ R₂, R₄, a, b, and d are as defined in Formula 1, Formula 2, and Formula 3.

11. The light emitting element of claim 1, wherein the amine compound represented by Formula 1 is represented by one of Formula 1-6-1 to Formula 1-6-4: 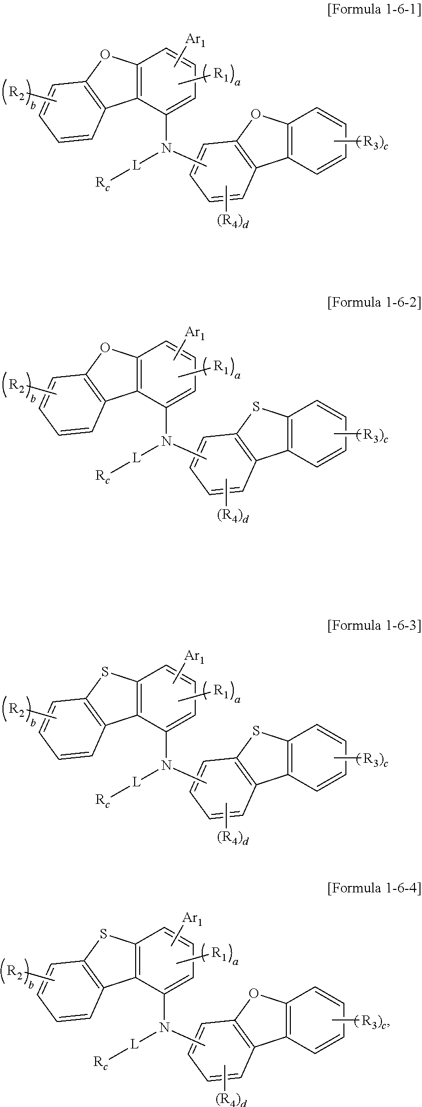
and
in Formula 1-6-1 to Formula 1-6-4,
R_{C}, L, Ar₁, R₁ to R₄, and a to d are as defined in Formula 1, Formula 2, and Formula 3.

12. The light emitting element of claim 1, wherein the amine compound represented by Formula 1 is represented by one of Formula 1-7-1 or Formula 1-7-2: 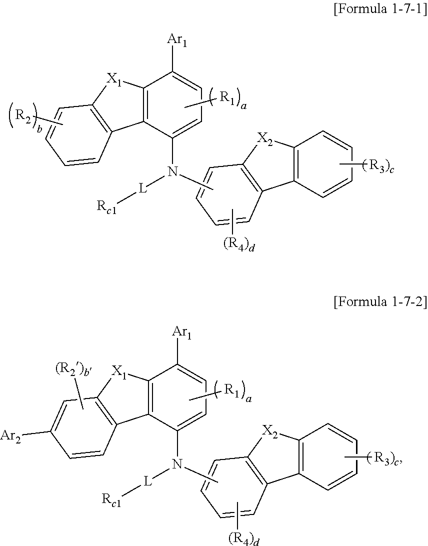
in Formula 1-7-1 and Formula 1-7-2,
R₂′ is a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Ar₂ is a substituted or unsubstituted aryl group of 6 to 30 ring-forming carbon atoms, or a substituted or unsubstituted heteroaryl group of 2 to 30 ring-forming carbon atoms,
b′ is an integer of 0 to 3, and
R_{C1} is selected from Compound Group A: 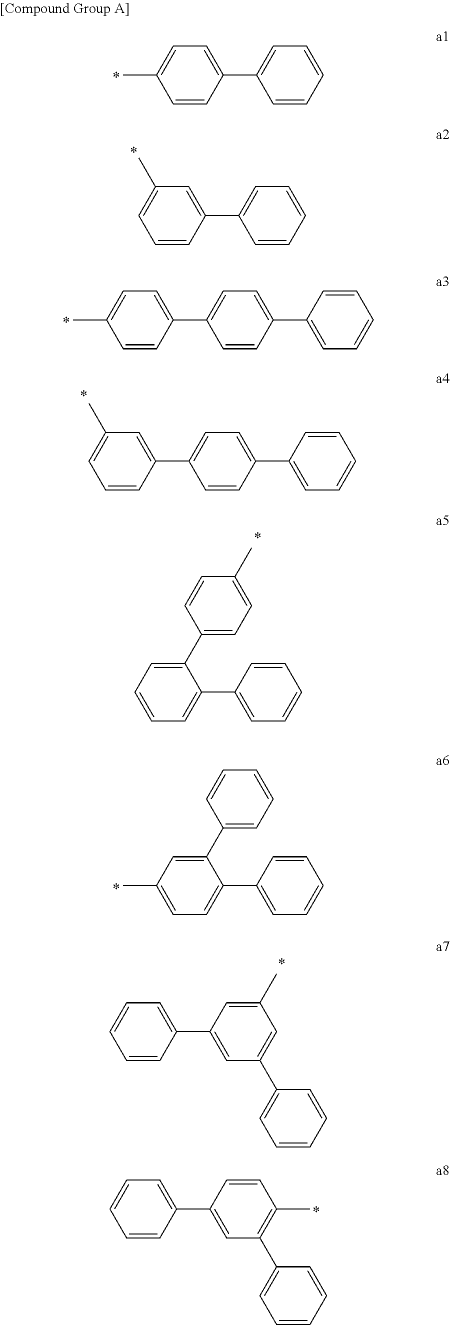 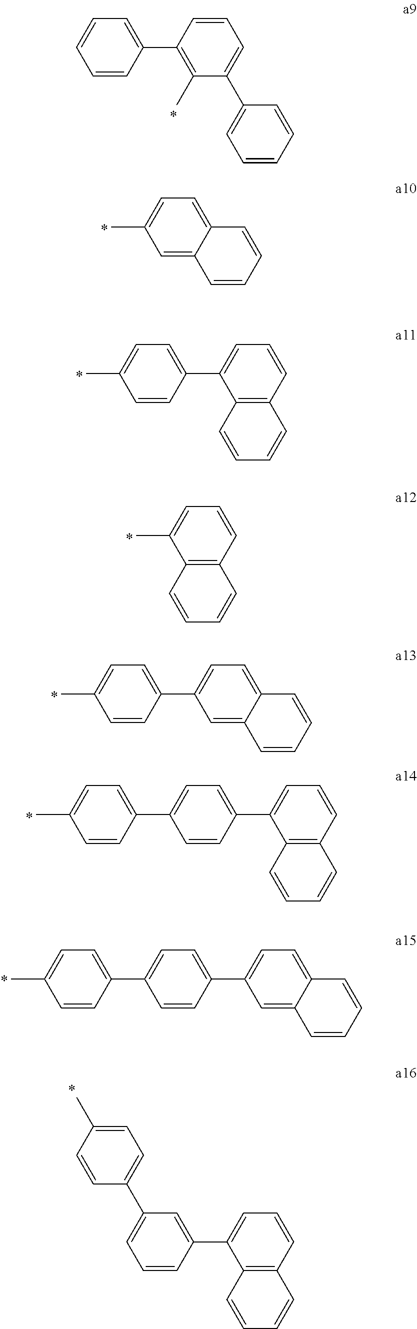 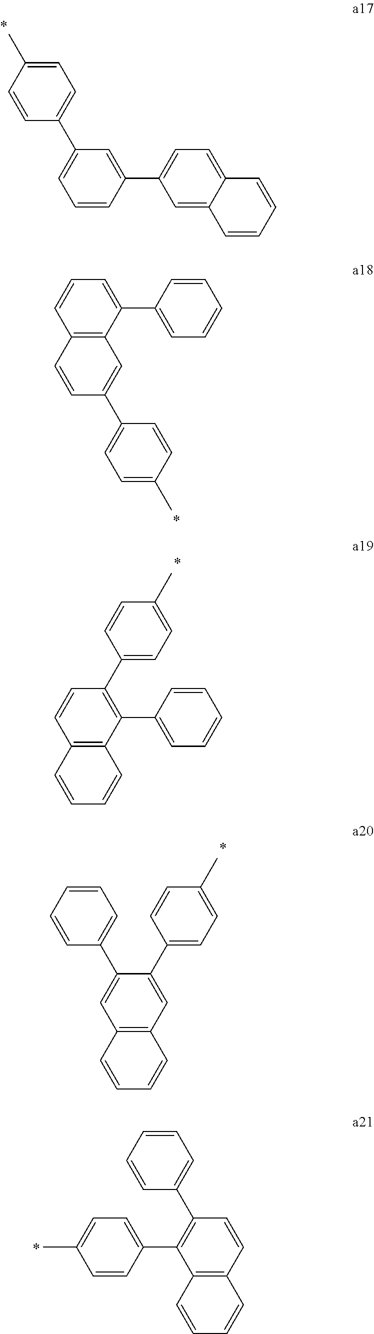 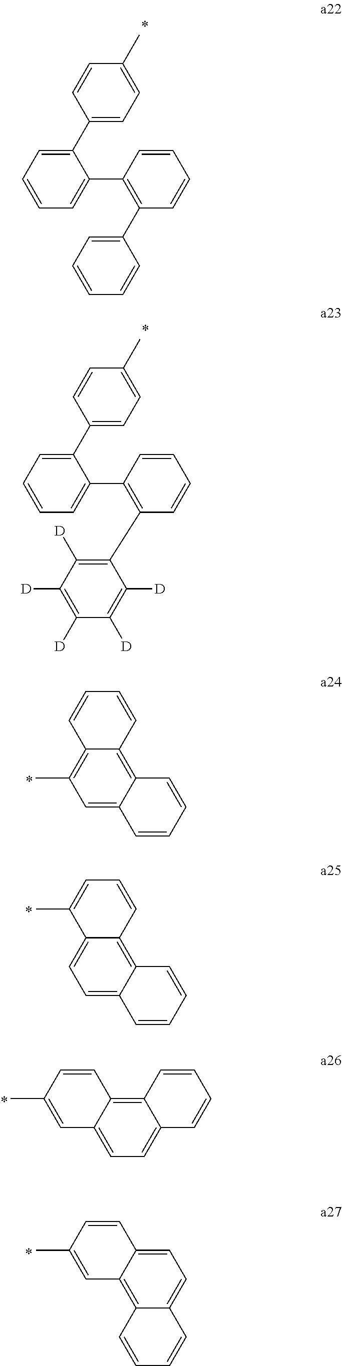 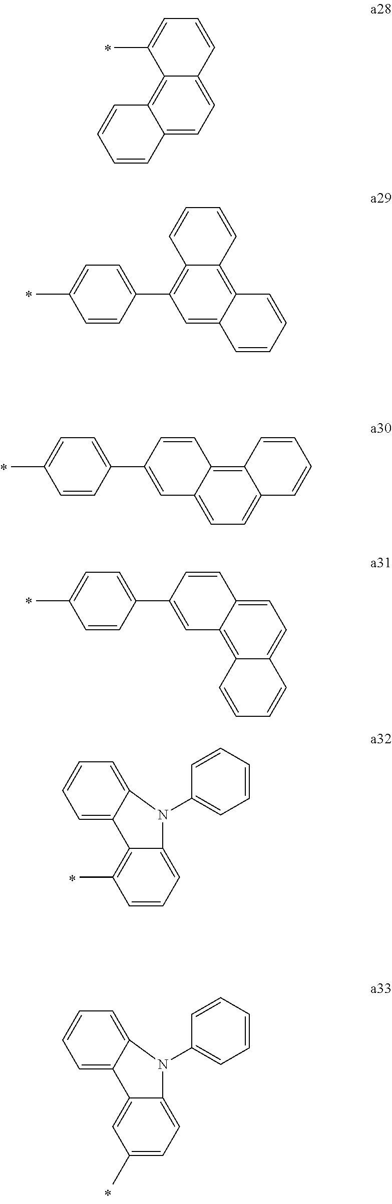 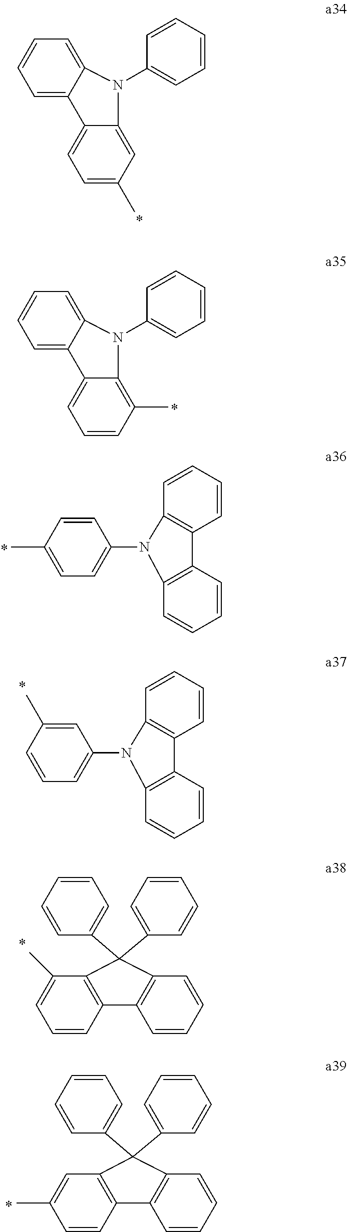 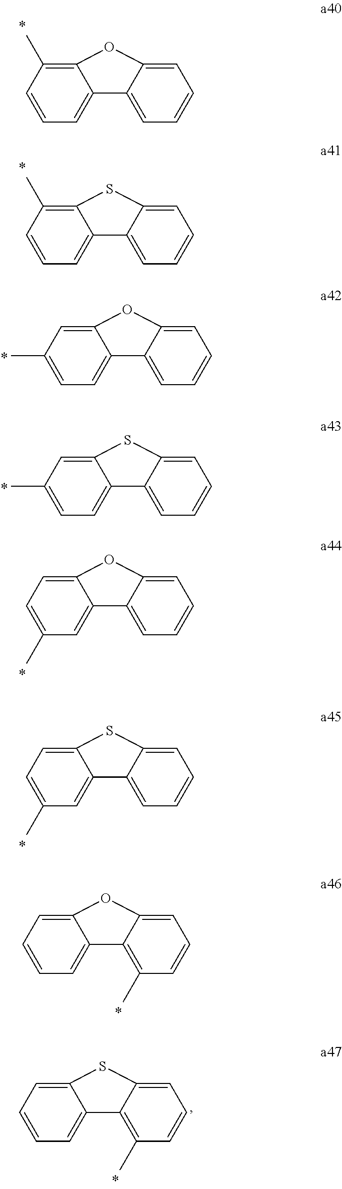
and
in Formula 1-7-1 and Formula 1-7-2,
Ar₁, X₁, X₂, R₁ to R₄, and a to d are as defined in Formula 1, Formula 2, and Formula 3.

13. The light emitting element of claim 1, wherein L is represented by one of Formula L-1 to Formula L-6: 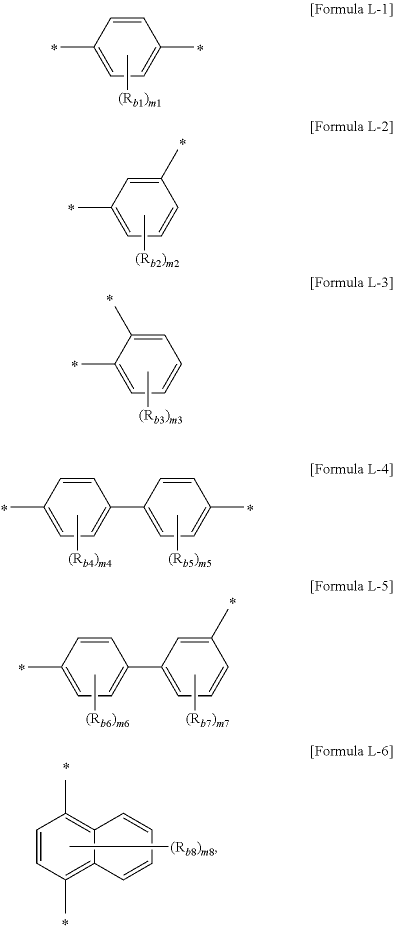
and
in Formula L-1 to Formula L-6,
R_{b1} to R_{b8} are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
m1 to m7 are each independently an integer of 0 to 4, and
m8 is an integer of 0 to 6.

14. The light emitting element of claim 1, wherein the amine compound represented by Formula 1 is selected from Compound Group 1:

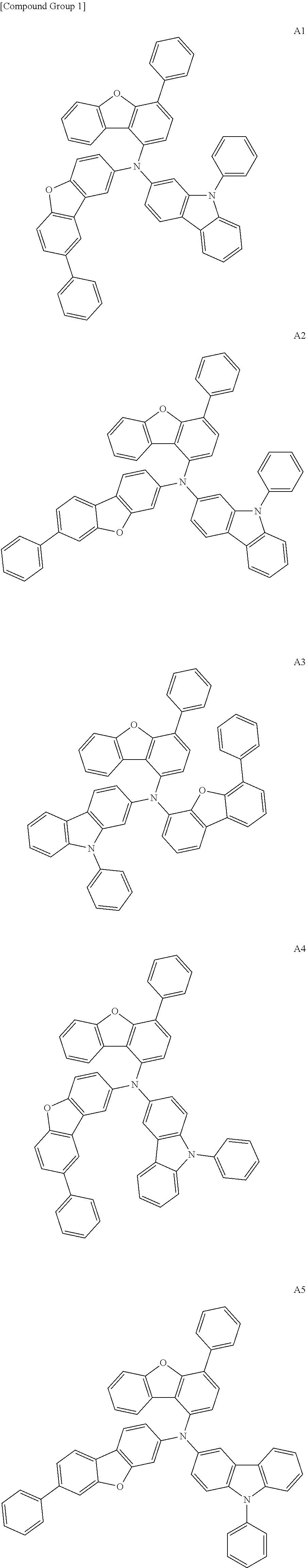 [SMILES: c1ccc(-c2ccc3oc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)oc2cc(N(c4ccc5c6ccccc6n(-c6ccccc6)c5c4)c4ccc(-c5ccccc5)c5oc6ccccc6c45)ccc23)cc1 ; c1ccc(-c2cccc3c2oc2c(N(c4ccc5c6ccccc6n(-c6ccccc6)c5c4)c4ccc(-c5ccccc5)c5oc6ccccc6c45)cccc23)cc1 ; c1ccc(-c2ccc3oc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)oc2cc(N(c4ccc5c(c4)c4ccccc4n5-c4ccccc4)c4ccc(-c5ccccc5)c5oc6ccccc6c45)ccc23)cc1]

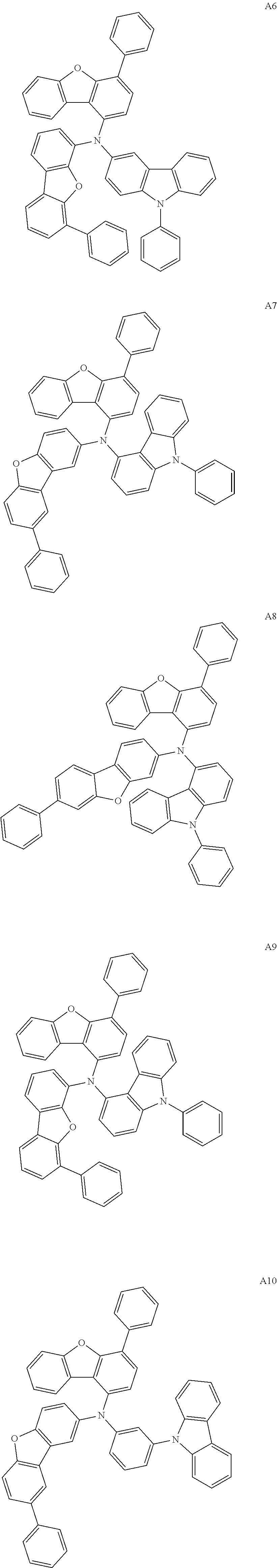 [SMILES: c1ccc(-c2cccc3c2oc2c(N(c4ccc5c(c4)c4ccccc4n5-c4ccccc4)c4ccc(-c5ccccc5)c5oc6ccccc6c45)cccc23)cc1 ; c1ccc(-c2ccc3oc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)oc2cc(N(c4ccc(-c5ccccc5)c5oc6ccccc6c45)c4cccc5c4c4ccccc4n5-c4ccccc4)ccc23)cc1 ; c1ccc(-c2cccc3c2oc2c(N(c4ccc(-c5ccccc5)c5oc6ccccc6c45)c4cccc5c4c4ccccc4n5-c4ccccc4)cccc23)cc1 ; c1ccc(-c2ccc3oc4ccc(N(c5cccc(-n6c7ccccc7c7ccccc76)c5)c5ccc(-c6ccccc6)c6oc7ccccc7c56)cc4c3c2)cc1]

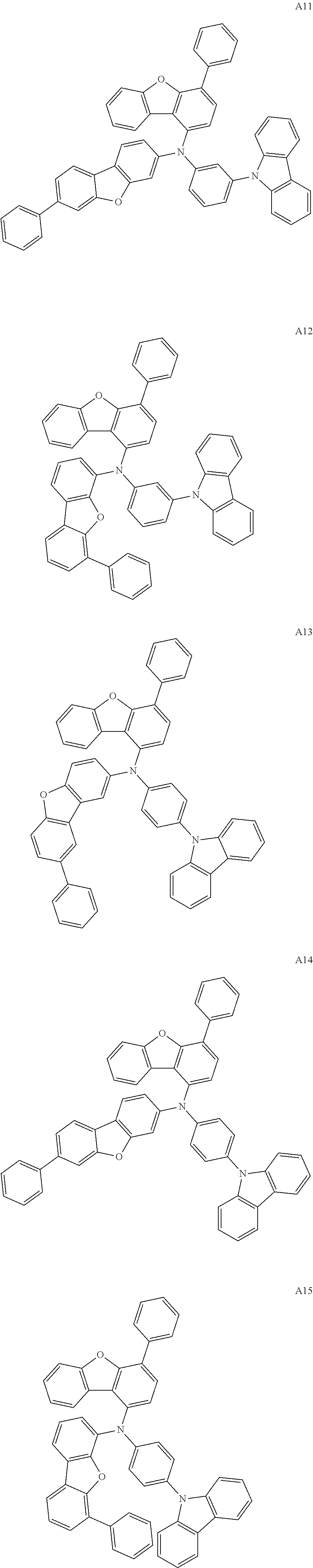 [SMILES: c1ccc(-c2ccc3c(c2)oc2cc(N(c4cccc(-n5c6ccccc6c6ccccc65)c4)c4ccc(-c5ccccc5)c5oc6ccccc6c45)ccc23)cc1 ; c1ccc(-c2cccc3c2oc2c(N(c4cccc(-n5c6ccccc6c6ccccc65)c4)c4ccc(-c5ccccc5)c5oc6ccccc6c45)cccc23)cc1 ; c1ccc(-c2ccc3oc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)oc2cc(N(c4ccc(-n5c6ccccc6c6ccccc65)cc4)c4ccc(-c5ccccc5)c5oc6ccccc6c45)ccc23)cc1 ; c1ccc(-c2cccc3c2oc2c(N(c4ccc(-n5c6ccccc6c6ccccc65)cc4)c4ccc(-c5ccccc5)c5oc6ccccc6c45)cccc23)cc1]

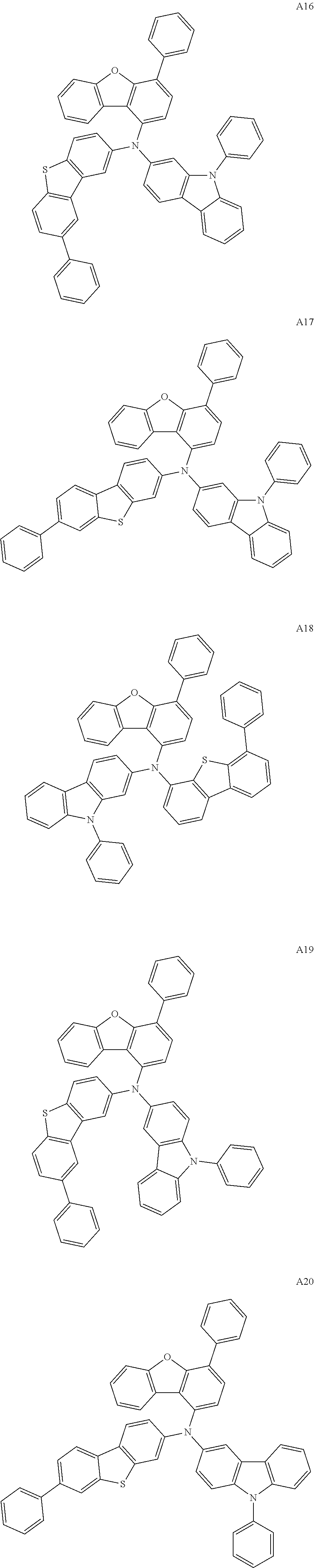 [SMILES: c1ccc(-c2ccc3sc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)sc2cc(N(c4ccc5c6ccccc6n(-c6ccccc6)c5c4)c4ccc(-c5ccccc5)c5oc6ccccc6c45)ccc23)cc1 ; c1ccc(-c2ccc(N(c3ccc4c5ccccc5n(-c5ccccc5)c4c3)c3cccc4c3sc3c(-c5ccccc5)cccc34)c3c2oc2ccccc23)cc1 ; c1ccc(-c2ccc3sc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)sc2cc(N(c4ccc5c(c4)c4ccccc4n5-c4ccccc4)c4ccc(-c5ccccc5)c5oc6ccccc6c45)ccc23)cc1]

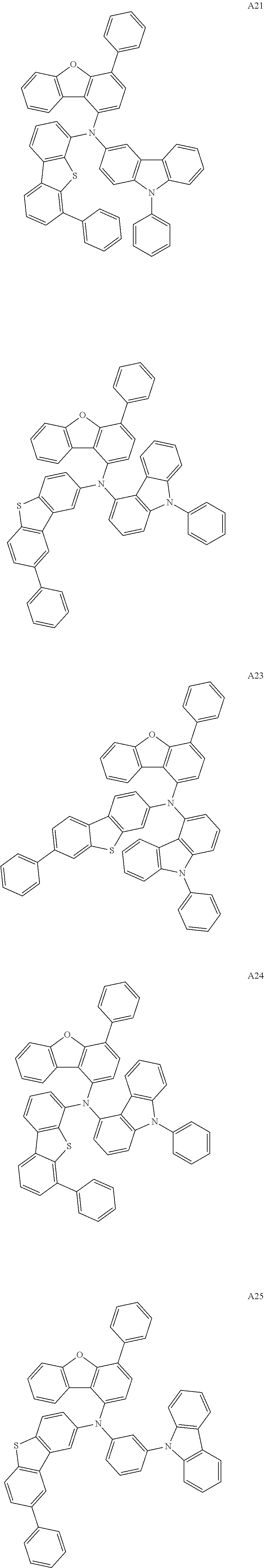 [SMILES: c1ccc(-c2ccc(N(c3ccc4c(c3)c3ccccc3n4-c3ccccc3)c3cccc4c3sc3c(-c5ccccc5)cccc34)c3c2oc2ccccc23)cc1 ; c1ccc(-c2ccc3sc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)sc2cc(N(c4ccc(-c5ccccc5)c5oc6ccccc6c45)c4cccc5c4c4ccccc4n5-c4ccccc4)ccc23)cc1 ; c1ccc(-c2ccc(N(c3cccc4c3sc3c(-c5ccccc5)cccc34)c3cccc4c3c3ccccc3n4-c3ccccc3)c3c2oc2ccccc23)cc1 ; c1ccc(-c2ccc3sc4ccc(N(c5cccc(-n6c7ccccc7c7ccccc76)c5)c5ccc(-c6ccccc6)c6oc7ccccc7c56)cc4c3c2)cc1]

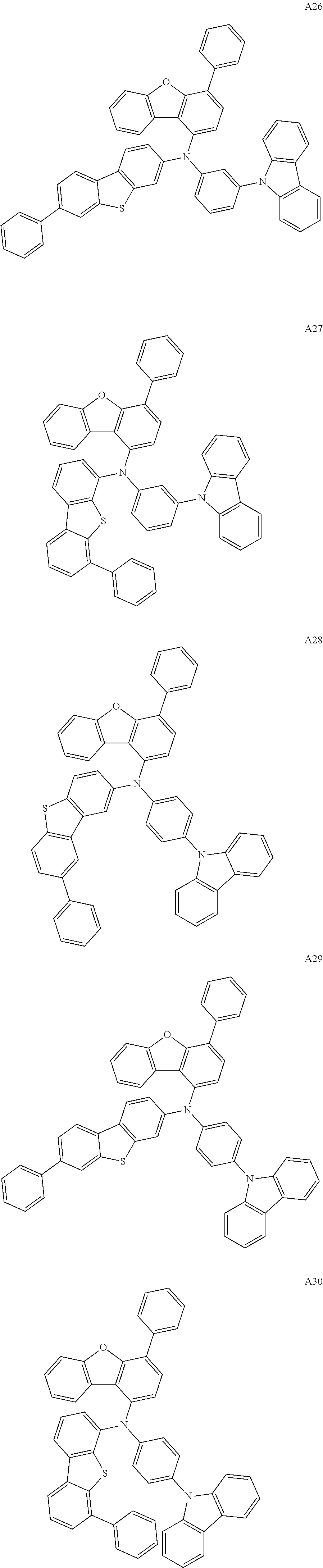 [SMILES: c1ccc(-c2ccc3c(c2)sc2cc(N(c4cccc(-n5c6ccccc6c6ccccc65)c4)c4ccc(-c5ccccc5)c5oc6ccccc6c45)ccc23)cc1 ; c1ccc(-c2ccc(N(c3cccc(-n4c5ccccc5c5ccccc54)c3)c3cccc4c3sc3c(-c5ccccc5)cccc34)c3c2oc2ccccc23)cc1 ; c1ccc(-c2ccc3sc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5oc6ccccc6c45)ccc23)cc1 ; c1ccc(-c2ccc(N(c3ccc(-n4c5ccccc5c5ccccc54)cc3)c3cccc4c3sc3c(-c5ccccc5)cccc34)c3c2oc2ccccc23)cc1]

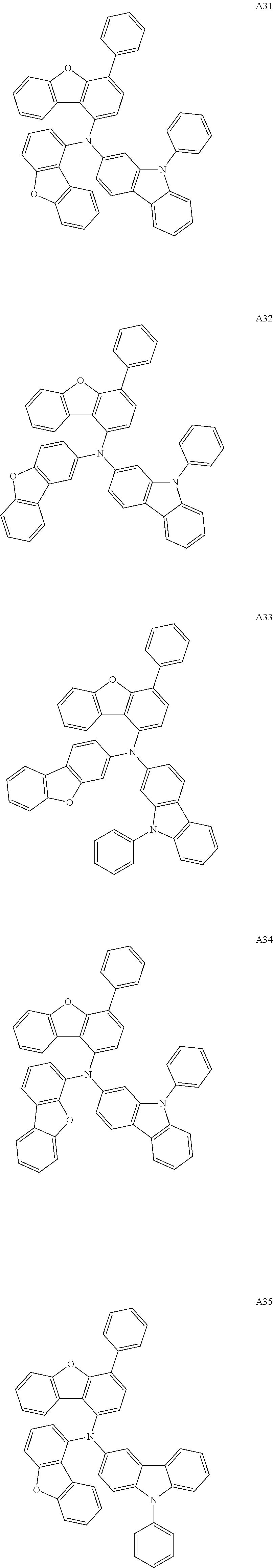 [SMILES: c1ccc(-c2ccc(N(c3ccc4c5ccccc5n(-c5ccccc5)c4c3)c3cccc4oc5ccccc5c34)c3c2oc2ccccc23)cc1 ; c1ccc(-c2ccc(N(c3ccc4oc5ccccc5c4c3)c3ccc4c5ccccc5n(-c5ccccc5)c4c3)c3c2oc2ccccc23)cc1 ; c1ccc(-c2ccc(N(c3ccc4c(c3)oc3ccccc34)c3ccc4c5ccccc5n(-c5ccccc5)c4c3)c3c2oc2ccccc23)cc1 ; c1ccc(-c2ccc(N(c3ccc4c5ccccc5n(-c5ccccc5)c4c3)c3cccc4c3oc3ccccc34)c3c2oc2ccccc23)cc1 ; c1ccc(-c2ccc(N(c3ccc4c(c3)c3ccccc3n4-c3ccccc3)c3cccc4oc5ccccc5c34)c3c2oc2ccccc23)cc1]

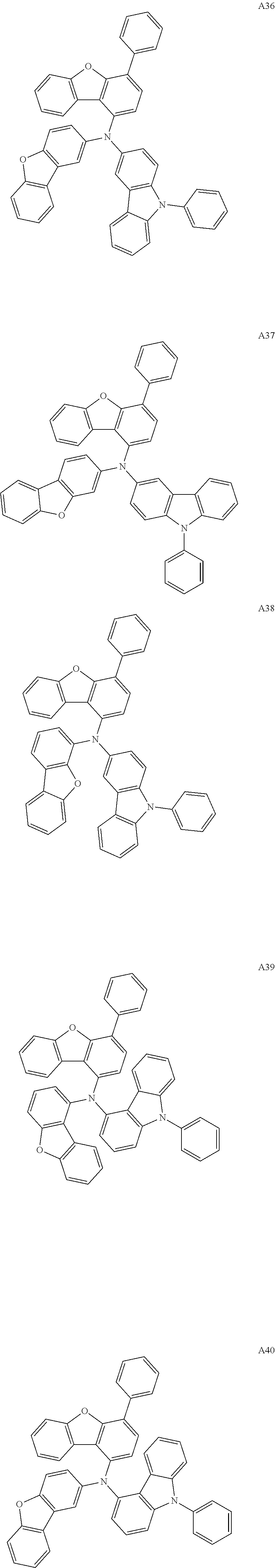 [SMILES: c1ccc(-c2ccc(N(c3ccc4oc5ccccc5c4c3)c3ccc4c(c3)c3ccccc3n4-c3ccccc3)c3c2oc2ccccc23)cc1 ; c1ccc(-c2ccc(N(c3ccc4c(c3)oc3ccccc34)c3ccc4c(c3)c3ccccc3n4-c3ccccc3)c3c2oc2ccccc23)cc1 ; c1ccc(-c2ccc(N(c3ccc4c(c3)c3ccccc3n4-c3ccccc3)c3cccc4c3oc3ccccc34)c3c2oc2ccccc23)cc1 ; c1ccc(-c2ccc(N(c3cccc4oc5ccccc5c34)c3cccc4c3c3ccccc3n4-c3ccccc3)c3c2oc2ccccc23)cc1 ; c1ccc(-c2ccc(N(c3ccc4oc5ccccc5c4c3)c3cccc4c3c3ccccc3n4-c3ccccc3)c3c2oc2ccccc23)cc1]

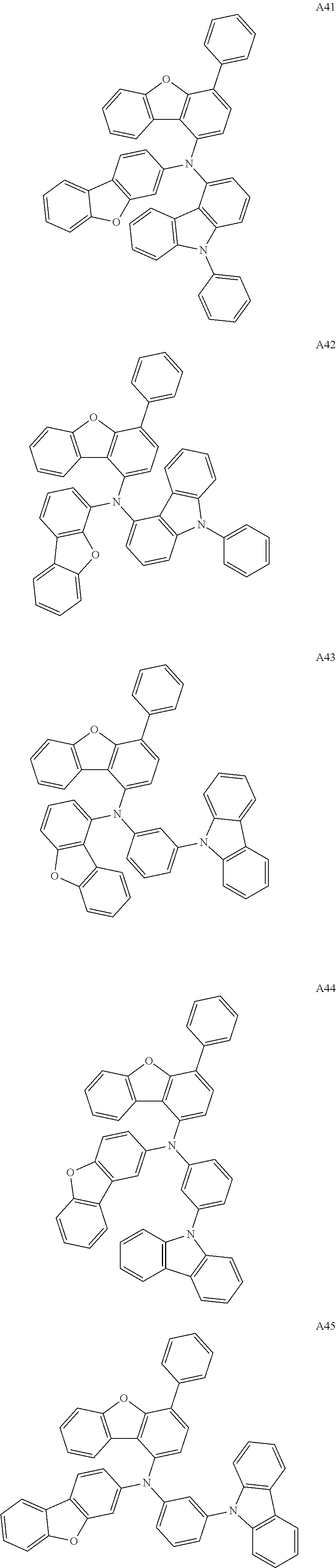

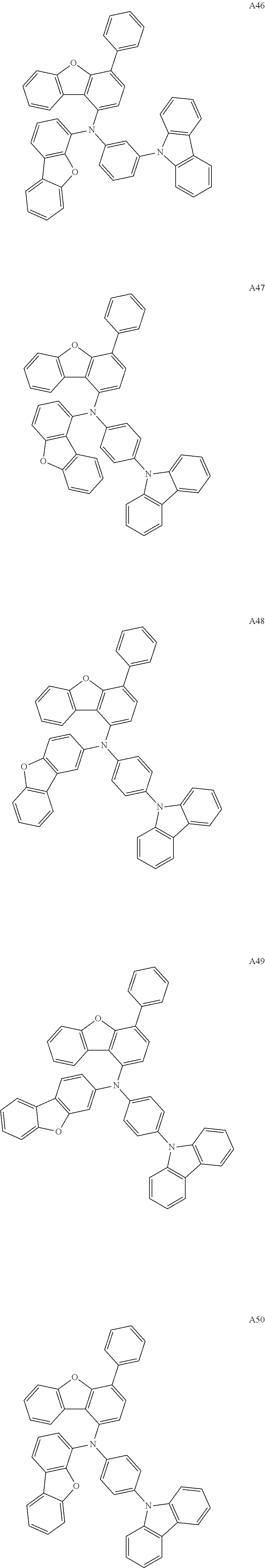 [SMILES: c1ccc(-c2ccc(N(c3cccc(-n4c5ccccc5c5ccccc54)c3)c3cccc4c3oc3ccccc34)c3c2oc2ccccc23)cc1 ; c1ccc(-c2ccc(N(c3ccc(-n4c5ccccc5c5ccccc54)cc3)c3cccc4oc5ccccc5c34)c3c2oc2ccccc23)cc1 ; c1ccc(-c2ccc(N(c3ccc(-n4c5ccccc5c5ccccc54)cc3)c3ccc4oc5ccccc5c4c3)c3c2oc2ccccc23)cc1 ; c1ccc(-c2ccc(N(c3ccc(-n4c5ccccc5c5ccccc54)cc3)c3ccc4c(c3)oc3ccccc34)c3c2oc2ccccc23)cc1 ; c1ccc(-c2ccc(N(c3ccc(-n4c5ccccc5c5ccccc54)cc3)c3cccc4c3oc3ccccc34)c3c2oc2ccccc23)cc1]

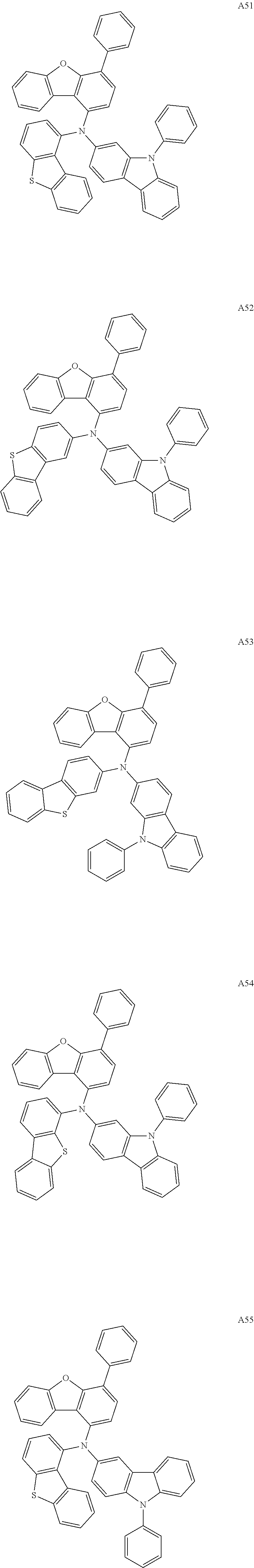 [SMILES: c1ccc(-c2ccc(N(c3ccc4c5ccccc5n(-c5ccccc5)c4c3)c3cccc4sc5ccccc5c34)c3c2oc2ccccc23)cc1 ; c1ccc(-c2ccc(N(c3ccc4sc5ccccc5c4c3)c3ccc4c5ccccc5n(-c5ccccc5)c4c3)c3c2oc2ccccc23)cc1 ; c1ccc(-c2ccc(N(c3ccc4c(c3)sc3ccccc34)c3ccc4c5ccccc5n(-c5ccccc5)c4c3)c3c2oc2ccccc23)cc1 ; c1ccc(-c2ccc(N(c3ccc4c5ccccc5n(-c5ccccc5)c4c3)c3cccc4c3sc3ccccc34)c3c2oc2ccccc23)cc1 ; c1ccc(-c2ccc(N(c3ccc4c(c3)c3ccccc3n4-c3ccccc3)c3cccc4sc5ccccc5c34)c3c2oc2ccccc23)cc1]

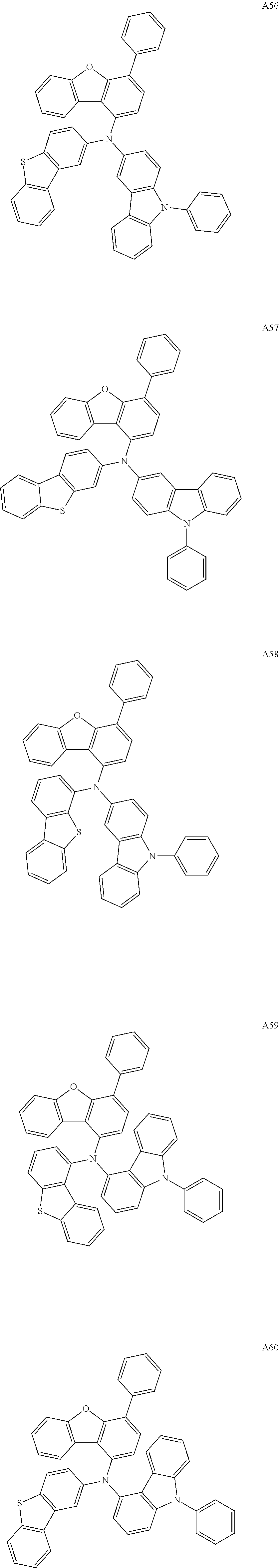 [SMILES: c1ccc(-c2ccc(N(c3ccc4sc5ccccc5c4c3)c3ccc4c(c3)c3ccccc3n4-c3ccccc3)c3c2oc2ccccc23)cc1 ; c1ccc(-c2ccc(N(c3ccc4c(c3)sc3ccccc34)c3ccc4c(c3)c3ccccc3n4-c3ccccc3)c3c2oc2ccccc23)cc1 ; c1ccc(-c2ccc(N(c3ccc4c(c3)c3ccccc3n4-c3ccccc3)c3cccc4c3sc3ccccc34)c3c2oc2ccccc23)cc1 ; c1ccc(-c2ccc(N(c3cccc4sc5ccccc5c34)c3cccc4c3c3ccccc3n4-c3ccccc3)c3c2oc2ccccc23)cc1 ; c1ccc(-c2ccc(N(c3ccc4sc5ccccc5c4c3)c3cccc4c3c3ccccc3n4-c3ccccc3)c3c2oc2ccccc23)cc1]

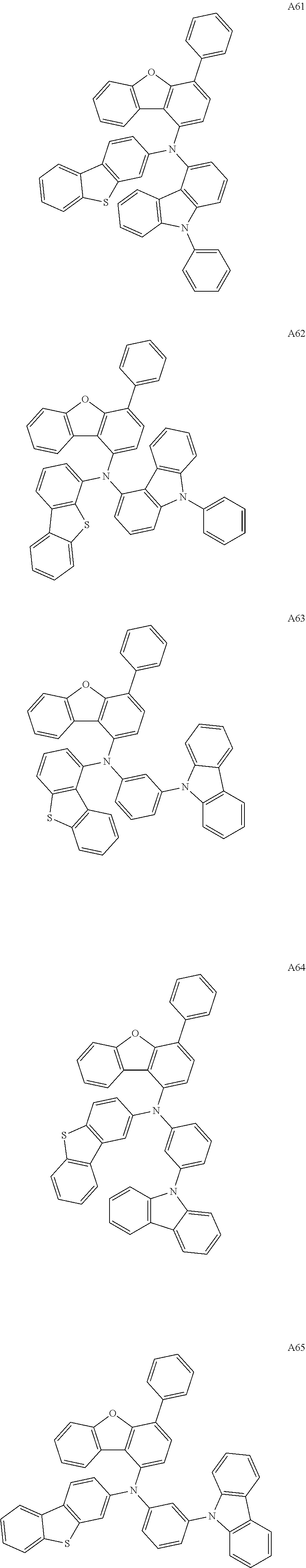 [SMILES: c1ccc(-c2ccc(N(c3ccc4c(c3)sc3ccccc34)c3cccc4c3c3ccccc3n4-c3ccccc3)c3c2oc2ccccc23)cc1 ; c1ccc(-c2ccc(N(c3cccc4c3sc3ccccc34)c3cccc4c3c3ccccc3n4-c3ccccc3)c3c2oc2ccccc23)cc1 ; c1ccc(-c2ccc(N(c3cccc(-n4c5ccccc5c5ccccc54)c3)c3cccc4sc5ccccc5c34)c3c2oc2ccccc23)cc1 ; c1ccc(-c2ccc(N(c3cccc(-n4c5ccccc5c5ccccc54)c3)c3ccc4sc5ccccc5c4c3)c3c2oc2ccccc23)cc1 ; c1ccc(-c2ccc(N(c3cccc(-n4c5ccccc5c5ccccc54)c3)c3ccc4c(c3)sc3ccccc34)c3c2oc2ccccc23)cc1]

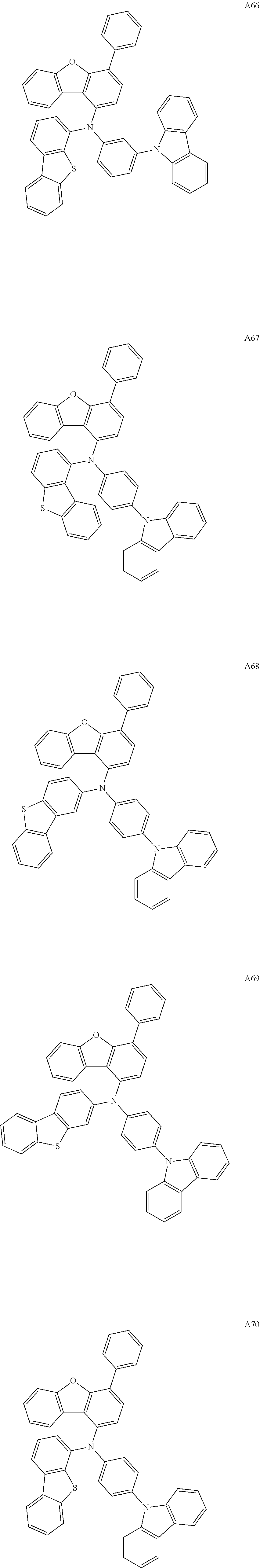 [SMILES: c1ccc(-c2ccc(N(c3cccc(-n4c5ccccc5c5ccccc54)c3)c3cccc4c3sc3ccccc34)c3c2oc2ccccc23)cc1 ; c1ccc(-c2ccc(N(c3ccc(-n4c5ccccc5c5ccccc54)cc3)c3cccc4sc5ccccc5c34)c3c2oc2ccccc23)cc1 ; c1ccc(-c2ccc(N(c3ccc(-n4c5ccccc5c5ccccc54)cc3)c3ccc4sc5ccccc5c4c3)c3c2oc2ccccc23)cc1 ; c1ccc(-c2ccc(N(c3ccc(-n4c5ccccc5c5ccccc54)cc3)c3ccc4c(c3)sc3ccccc34)c3c2oc2ccccc23)cc1 ; c1ccc(-c2ccc(N(c3ccc(-n4c5ccccc5c5ccccc54)cc3)c3cccc4c3sc3ccccc34)c3c2oc2ccccc23)cc1]

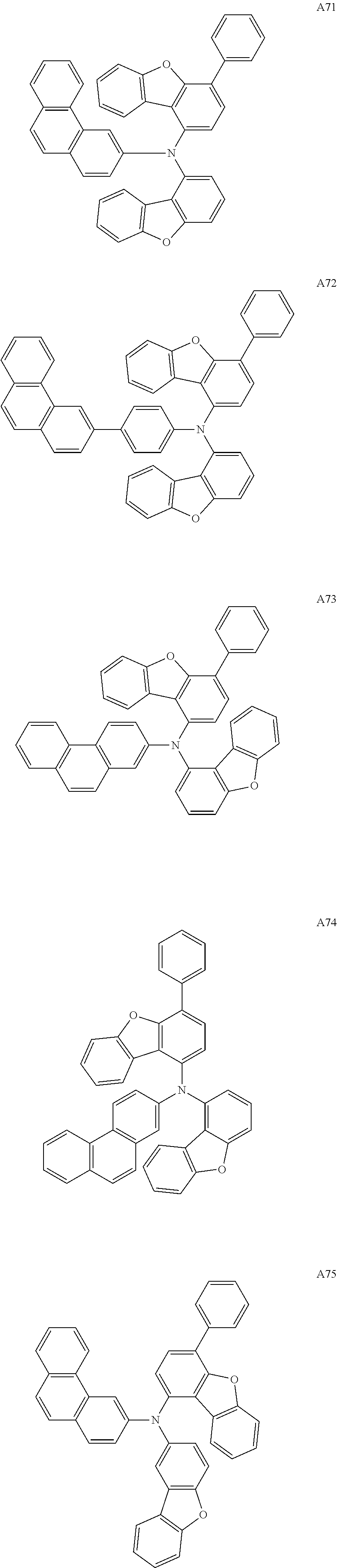 [SMILES: c1ccc(-c2ccc(N(c3ccc4ccc5ccccc5c4c3)c3cccc4oc5ccccc5c34)c3c2oc2ccccc23)cc1 ; c1ccc(-c2ccc(N(c3ccc(-c4ccc5ccc6ccccc6c5c4)cc3)c3cccc4oc5ccccc5c34)c3c2oc2ccccc23)cc1 ; c1ccc(-c2ccc(N(c3ccc4c(ccc5ccccc54)c3)c3cccc4oc5ccccc5c34)c3c2oc2ccccc23)cc1 ; c1ccc(-c2ccc(N(c3ccc4c(ccc5ccccc54)c3)c3cccc4oc5ccccc5c34)c3c2oc2ccccc23)cc1 ; c1ccc(-c2ccc(N(c3ccc4ccc5ccccc5c4c3)c3ccc4oc5ccccc5c4c3)c3c2oc2ccccc23)cc1]

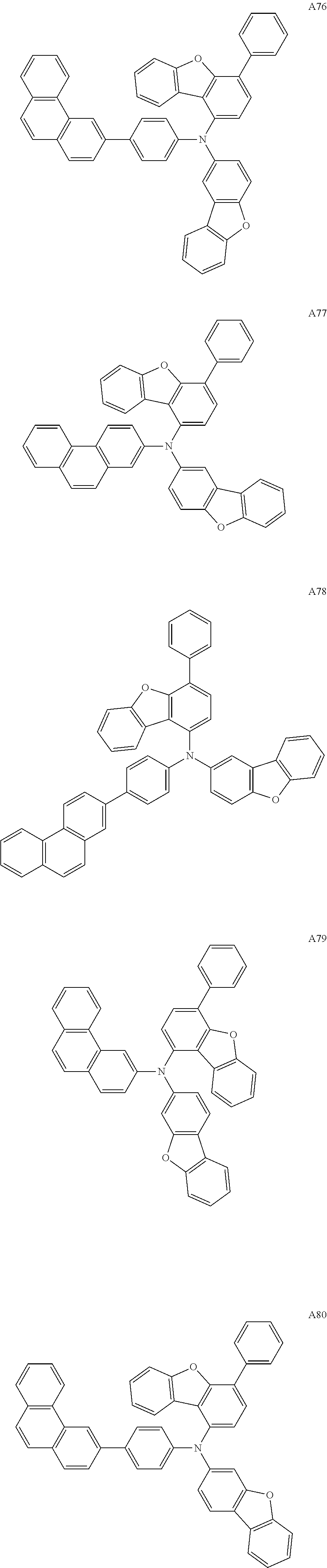 [SMILES: c1ccc(-c2ccc(N(c3ccc(-c4ccc5ccc6ccccc6c5c4)cc3)c3ccc4oc5ccccc5c4c3)c3c2oc2ccccc23)cc1 ; c1ccc(-c2ccc(N(c3ccc4c(ccc5ccccc54)c3)c3ccc4oc5ccccc5c4c3)c3c2oc2ccccc23)cc1 ; c1ccc(-c2ccc(N(c3ccc(-c4ccc5c(ccc6ccccc65)c4)cc3)c3ccc4oc5ccccc5c4c3)c3c2oc2ccccc23)cc1 ; c1ccc(-c2ccc(N(c3ccc4c(c3)oc3ccccc34)c3ccc4ccc5ccccc5c4c3)c3c2oc2ccccc23)cc1 ; c1ccc(-c2ccc(N(c3ccc(-c4ccc5ccc6ccccc6c5c4)cc3)c3ccc4c(c3)oc3ccccc34)c3c2oc2ccccc23)cc1]

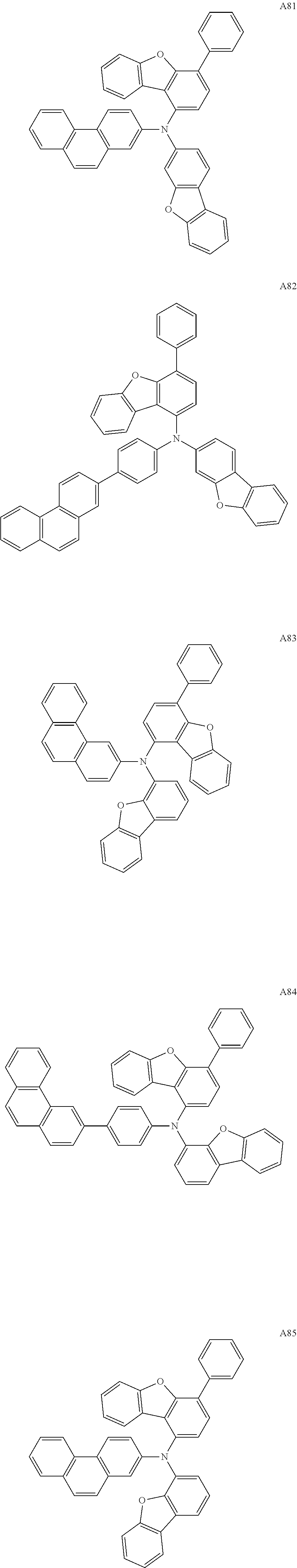 [SMILES: c1ccc(-c2ccc(N(c3ccc4c(ccc5ccccc54)c3)c3ccc4c(c3)oc3ccccc34)c3c2oc2ccccc23)cc1 ; c1ccc(-c2ccc(N(c3ccc(-c4ccc5c(ccc6ccccc65)c4)cc3)c3ccc4c(c3)oc3ccccc34)c3c2oc2ccccc23)cc1 ; c1ccc(-c2ccc(N(c3ccc4ccc5ccccc5c4c3)c3cccc4c3oc3ccccc34)c3c2oc2ccccc23)cc1 ; c1ccc(-c2ccc(N(c3ccc(-c4ccc5ccc6ccccc6c5c4)cc3)c3cccc4c3oc3ccccc34)c3c2oc2ccccc23)cc1 ; c1ccc(-c2ccc(N(c3ccc4c(ccc5ccccc54)c3)c3cccc4c3oc3ccccc34)c3c2oc2ccccc23)cc1]

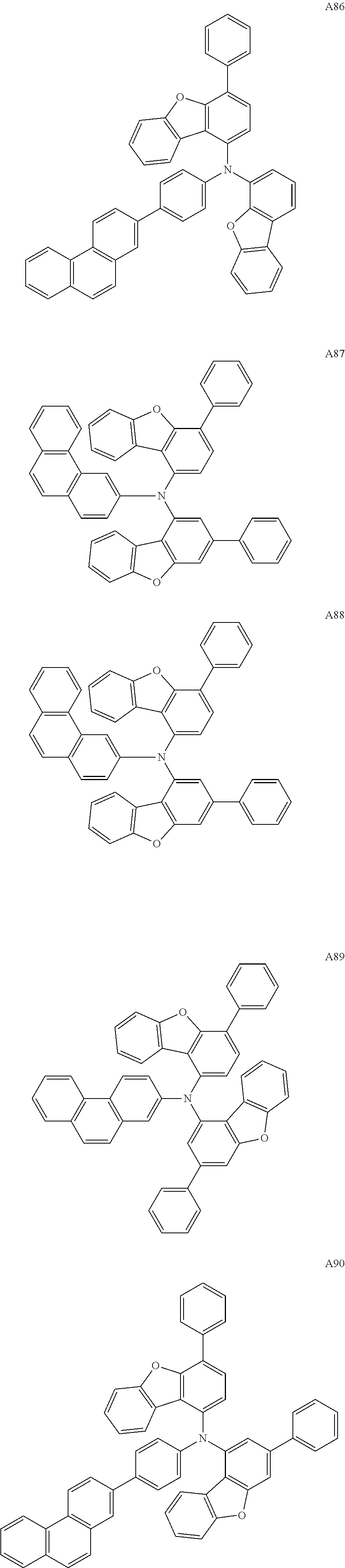 [SMILES: c1ccc(-c2ccc(N(c3ccc(-c4ccc5c(ccc6ccccc65)c4)cc3)c3cccc4c3oc3ccccc34)c3c2oc2ccccc23)cc1 ; c1ccc(-c2cc(N(c3ccc4ccc5ccccc5c4c3)c3ccc(-c4ccccc4)c4oc5ccccc5c34)c3c(c2)oc2ccccc23)cc1 ; c1ccc(-c2cc(N(c3ccc4ccc5ccccc5c4c3)c3ccc(-c4ccccc4)c4oc5ccccc5c34)c3c(c2)oc2ccccc23)cc1 ; c1ccc(-c2cc(N(c3ccc4c(ccc5ccccc54)c3)c3ccc(-c4ccccc4)c4oc5ccccc5c34)c3c(c2)oc2ccccc23)cc1 ; c1ccc(-c2cc(N(c3ccc(-c4ccc5c(ccc6ccccc65)c4)cc3)c3ccc(-c4ccccc4)c4oc5ccccc5c34)c3c(c2)oc2ccccc23)cc1]

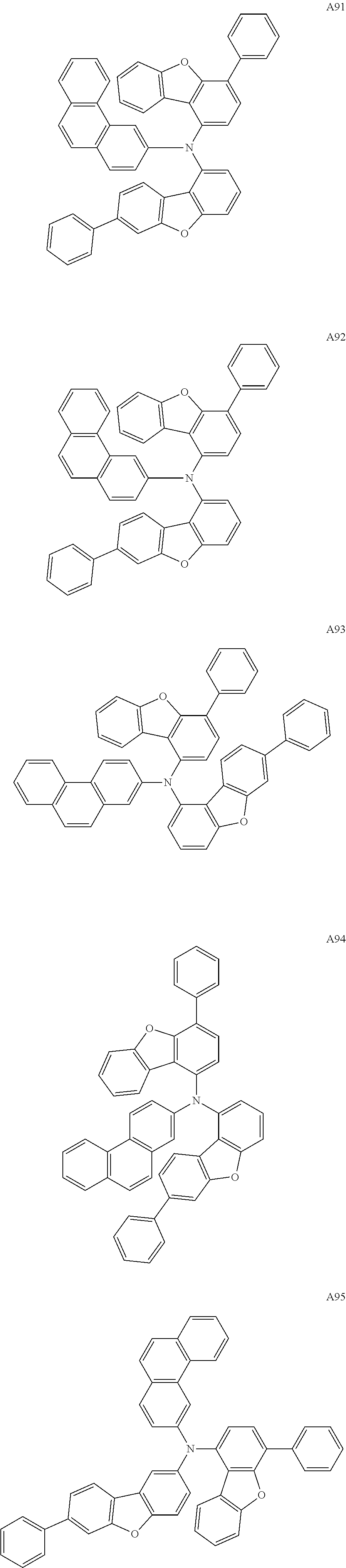 [SMILES: c1ccc(-c2ccc3c(c2)oc2cccc(N(c4ccc5ccc6ccccc6c5c4)c4ccc(-c5ccccc5)c5oc6ccccc6c45)c23)cc1 ; c1ccc(-c2ccc3c(c2)oc2cccc(N(c4ccc5ccc6ccccc6c5c4)c4ccc(-c5ccccc5)c5oc6ccccc6c45)c23)cc1 ; c1ccc(-c2ccc3c(c2)oc2cccc(N(c4ccc5c(ccc6ccccc65)c4)c4ccc(-c5ccccc5)c5oc6ccccc6c45)c23)cc1 ; c1ccc(-c2ccc3c(c2)oc2cccc(N(c4ccc5c(ccc6ccccc65)c4)c4ccc(-c5ccccc5)c5oc6ccccc6c45)c23)cc1 ; c1ccc(-c2ccc3c(c2)oc2ccc(N(c4ccc5ccc6ccccc6c5c4)c4ccc(-c5ccccc5)c5oc6ccccc6c45)cc23)cc1]

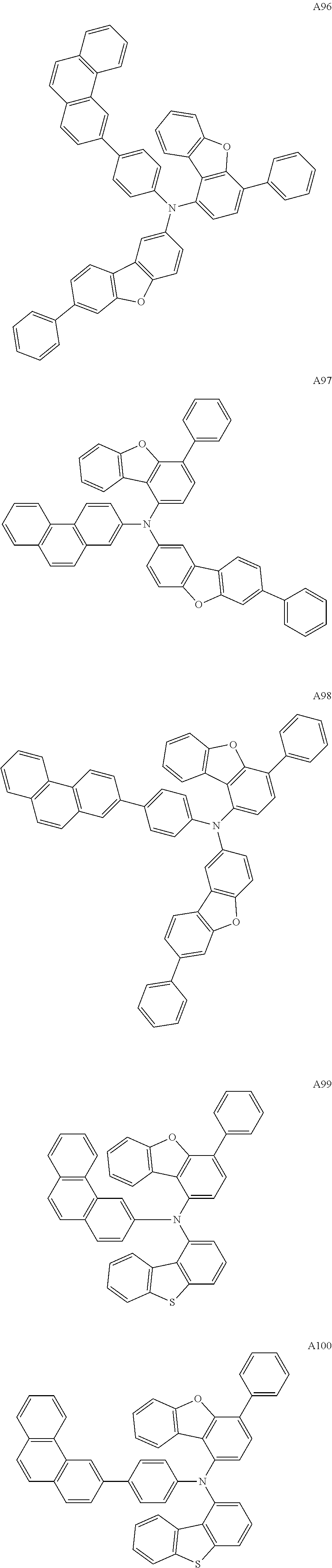 [SMILES: c1ccc(-c2ccc3c(c2)oc2ccc(N(c4ccc(-c5ccc6ccc7ccccc7c6c5)cc4)c4ccc(-c5ccccc5)c5oc6ccccc6c45)cc23)cc1 ; c1ccc(-c2ccc3c(c2)oc2ccc(N(c4ccc5c(ccc6ccccc65)c4)c4ccc(-c5ccccc5)c5oc6ccccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5oc6ccccc6c45)cc23)cc1 ; c1ccc(-c2ccc(N(c3ccc4ccc5ccccc5c4c3)c3cccc4sc5ccccc5c34)c3c2oc2ccccc23)cc1 ; c1ccc(-c2ccc(N(c3ccc(-c4ccc5ccc6ccccc6c5c4)cc3)c3cccc4sc5ccccc5c34)c3c2oc2ccccc23)cc1]

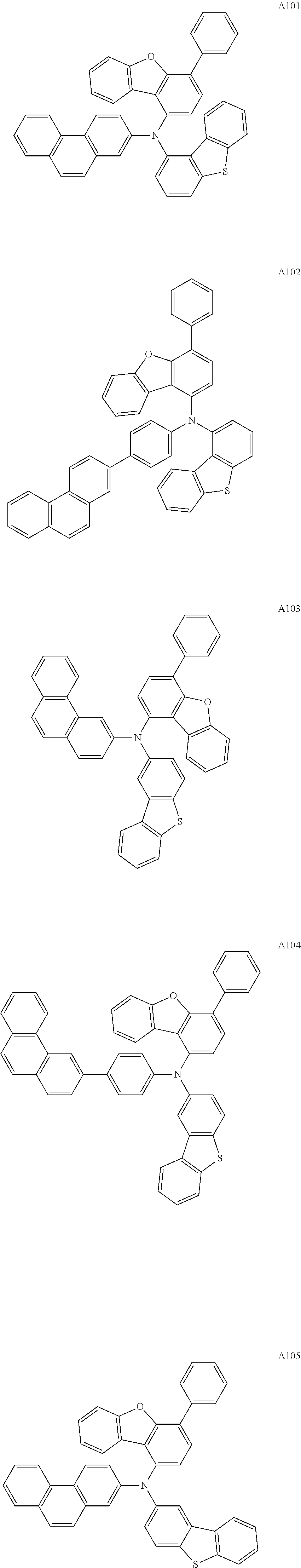 [SMILES: c1ccc(-c2ccc(N(c3ccc4c(ccc5ccccc54)c3)c3cccc4sc5ccccc5c34)c3c2oc2ccccc23)cc1 ; c1ccc(-c2ccc(N(c3ccc(-c4ccc5c(ccc6ccccc65)c4)cc3)c3cccc4sc5ccccc5c34)c3c2oc2ccccc23)cc1 ; c1ccc(-c2ccc(N(c3ccc4ccc5ccccc5c4c3)c3ccc4sc5ccccc5c4c3)c3c2oc2ccccc23)cc1 ; c1ccc(-c2ccc(N(c3ccc(-c4ccc5ccc6ccccc6c5c4)cc3)c3ccc4sc5ccccc5c4c3)c3c2oc2ccccc23)cc1 ; c1ccc(-c2ccc(N(c3ccc4c(ccc5ccccc54)c3)c3ccc4sc5ccccc5c4c3)c3c2oc2ccccc23)cc1]

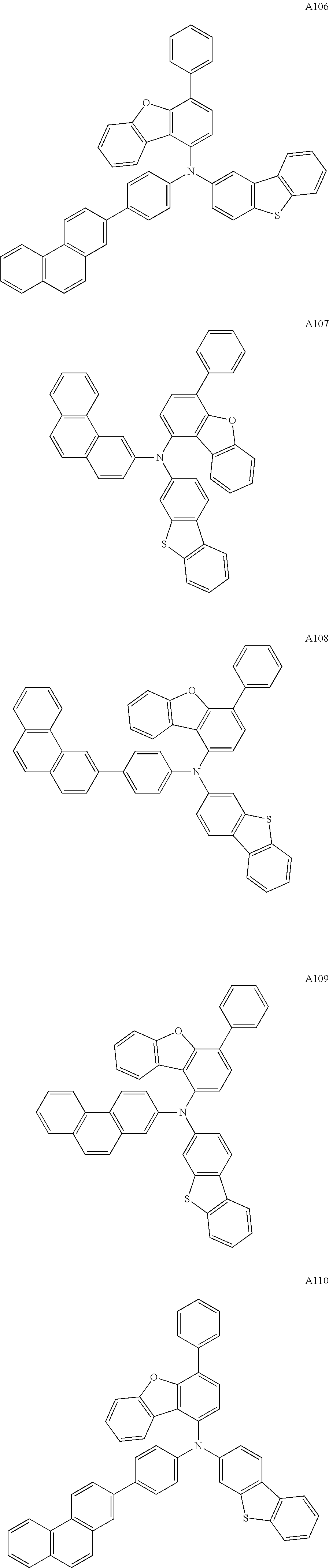 [SMILES: c1ccc(-c2ccc(N(c3ccc(-c4ccc5c(ccc6ccccc65)c4)cc3)c3ccc4sc5ccccc5c4c3)c3c2oc2ccccc23)cc1 ; c1ccc(-c2ccc(N(c3ccc4c(c3)sc3ccccc34)c3ccc4ccc5ccccc5c4c3)c3c2oc2ccccc23)cc1 ; c1ccc(-c2ccc(N(c3ccc(-c4ccc5ccc6ccccc6c5c4)cc3)c3ccc4c(c3)sc3ccccc34)c3c2oc2ccccc23)cc1 ; c1ccc(-c2ccc(N(c3ccc4c(ccc5ccccc54)c3)c3ccc4c(c3)sc3ccccc34)c3c2oc2ccccc23)cc1 ; c1ccc(-c2ccc(N(c3ccc(-c4ccc5c(ccc6ccccc65)c4)cc3)c3ccc4c(c3)sc3ccccc34)c3c2oc2ccccc23)cc1]

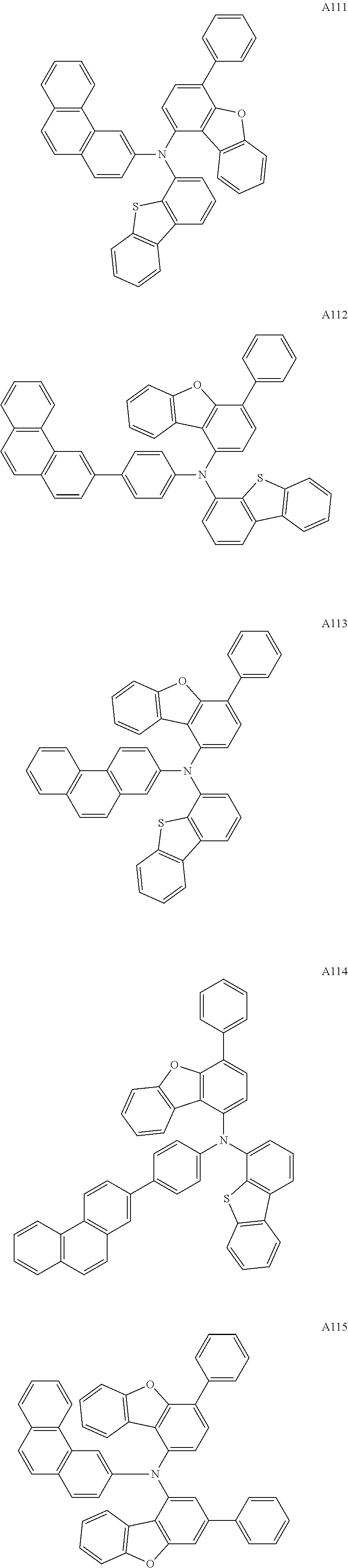 [SMILES: c1ccc(-c2ccc(N(c3ccc4ccc5ccccc5c4c3)c3cccc4c3sc3ccccc34)c3c2oc2ccccc23)cc1 ; c1ccc(-c2ccc(N(c3ccc(-c4ccc5ccc6ccccc6c5c4)cc3)c3cccc4c3sc3ccccc34)c3c2oc2ccccc23)cc1 ; c1ccc(-c2ccc(N(c3ccc4c(ccc5ccccc54)c3)c3cccc4c3sc3ccccc34)c3c2oc2ccccc23)cc1 ; c1ccc(-c2ccc(N(c3ccc(-c4ccc5c(ccc6ccccc65)c4)cc3)c3cccc4c3sc3ccccc34)c3c2oc2ccccc23)cc1 ; c1ccc(-c2cc(N(c3ccc4ccc5ccccc5c4c3)c3ccc(-c4ccccc4)c4oc5ccccc5c34)c3c(c2)oc2ccccc23)cc1]

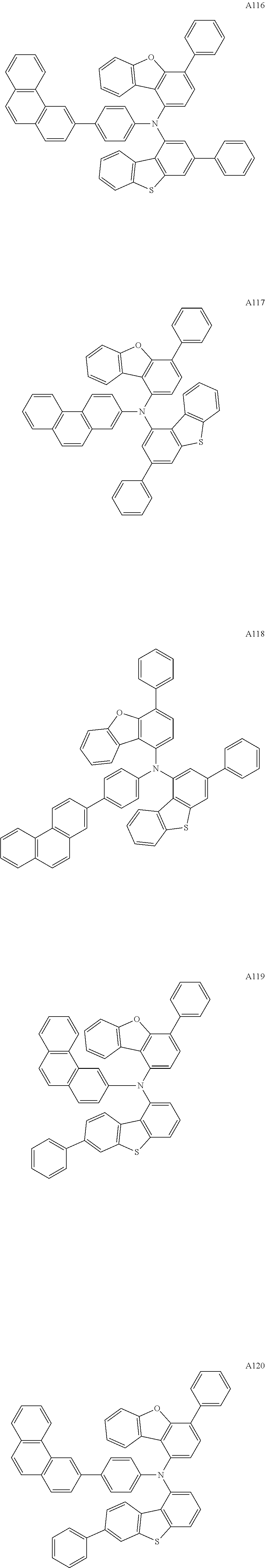 [SMILES: c1ccc(-c2cc(N(c3ccc(-c4ccc5ccc6ccccc6c5c4)cc3)c3ccc(-c4ccccc4)c4oc5ccccc5c34)c3c(c2)sc2ccccc23)cc1 ; c1ccc(-c2cc(N(c3ccc4c(ccc5ccccc54)c3)c3ccc(-c4ccccc4)c4oc5ccccc5c34)c3c(c2)sc2ccccc23)cc1 ; c1ccc(-c2cc(N(c3ccc(-c4ccc5c(ccc6ccccc65)c4)cc3)c3ccc(-c4ccccc4)c4oc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5oc6ccccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5oc6ccccc6c45)c23)cc1]

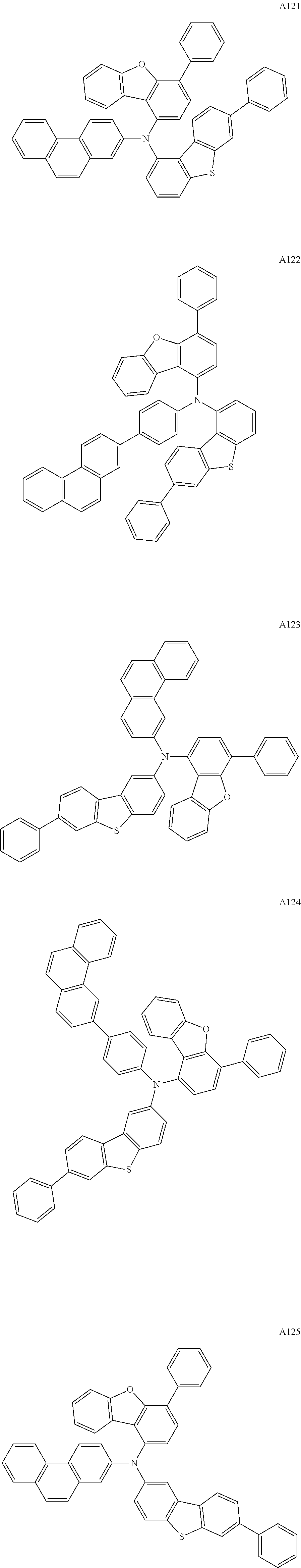 [SMILES: c1ccc(-c2ccc3c(c2)sc2cccc(N(c4ccc5c(ccc6ccccc65)c4)c4ccc(-c5ccccc5)c5oc6ccccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5oc6ccccc6c45)c23)cc1 ; c1ccc(-c2ccc3c(c2)sc2ccc(N(c4ccc5ccc6ccccc6c5c4)c4ccc(-c5ccccc5)c5oc6ccccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5oc6ccccc6c45)cc23)cc1 ; c1ccc(-c2ccc3c(c2)sc2ccc(N(c4ccc5c(ccc6ccccc65)c4)c4ccc(-c5ccccc5)c5oc6ccccc6c45)cc23)cc1]

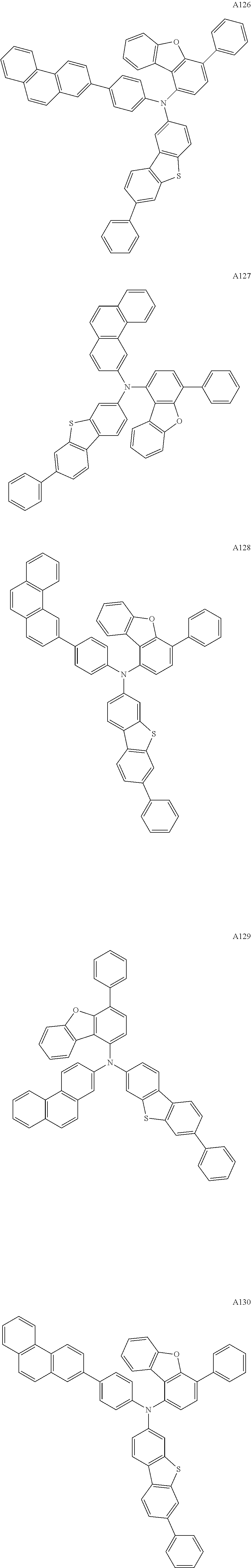 [SMILES: c1ccc(-c2ccc3c(c2)sc2ccc(N(c4ccc(-c5ccc6c(ccc7ccccc76)c5)cc4)c4ccc(-c5ccccc5)c5oc6ccccc6c45)cc23)cc1 ; c1ccc(-c2ccc3c(c2)sc2cc(N(c4ccc5ccc6ccccc6c5c4)c4ccc(-c5ccccc5)c5oc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5oc6ccccc6c45)ccc23)cc1 ; c1ccc(-c2ccc3c(c2)sc2cc(N(c4ccc5c(ccc6ccccc65)c4)c4ccc(-c5ccccc5)c5oc6ccccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5oc6ccccc6c45)ccc23)cc1]

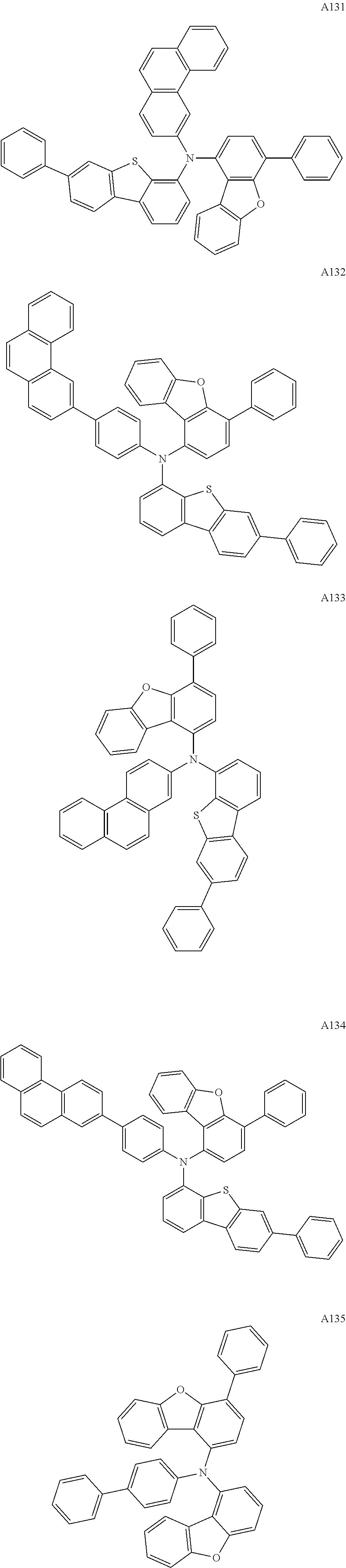 [SMILES: c1ccc(-c2ccc3c(c2)sc2c(N(c4ccc5ccc6ccccc6c5c4)c4ccc(-c5ccccc5)c5oc6ccccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5oc6ccccc6c45)cccc23)cc1 ; c1ccc(-c2ccc3c(c2)sc2c(N(c4ccc5c(ccc6ccccc65)c4)c4ccc(-c5ccccc5)c5oc6ccccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5oc6ccccc6c45)cccc23)cc1 ; c1ccc(-c2ccc(N(c3cccc4oc5ccccc5c34)c3ccc(-c4ccccc4)c4oc5ccccc5c34)cc2)cc1]

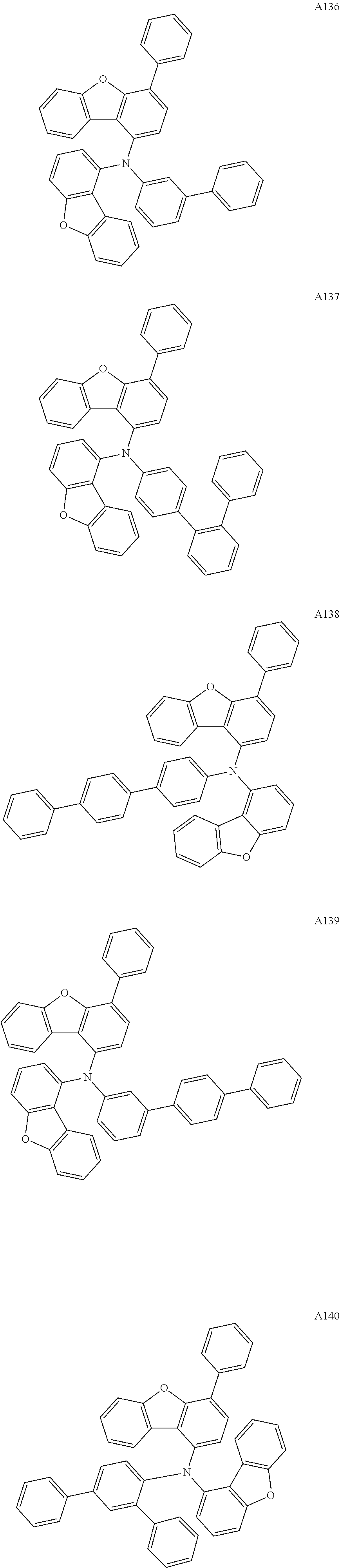 [SMILES: c1ccc(-c2cccc(N(c3cccc4oc5ccccc5c34)c3ccc(-c4ccccc4)c4oc5ccccc5c34)c2)cc1 ; c1ccc(-c2ccccc2-c2ccc(N(c3cccc4oc5ccccc5c34)c3ccc(-c4ccccc4)c4oc5ccccc5c34)cc2)cc1 ; c1ccc(-c2ccc(-c3ccc(N(c4cccc5oc6ccccc6c45)c4ccc(-c5ccccc5)c5oc6ccccc6c45)cc3)cc2)cc1 ; c1ccc(-c2ccc(-c3cccc(N(c4cccc5oc6ccccc6c45)c4ccc(-c5ccccc5)c5oc6ccccc6c45)c3)cc2)cc1 ; c1ccc(-c2ccc(N(c3cccc4oc5ccccc5c34)c3ccc(-c4ccccc4)c4oc5ccccc5c34)c(-c3ccccc3)c2)cc1]

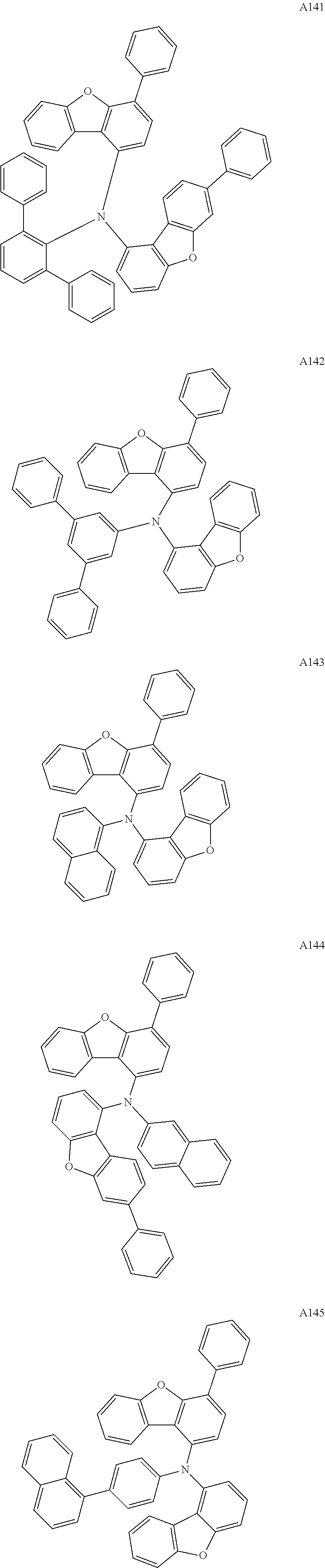 [SMILES: c1ccc(-c2ccc3c(c2)oc2cccc(N(c4c(-c5ccccc5)cccc4-c4ccccc4)c4ccc(-c5ccccc5)c5oc6ccccc6c45)c23)cc1 ; c1ccc(-c2cc(-c3ccccc3)cc(N(c3cccc4oc5ccccc5c34)c3ccc(-c4ccccc4)c4oc5ccccc5c34)c2)cc1 ; c1ccc(-c2ccc(N(c3cccc4ccccc34)c3cccc4oc5ccccc5c34)c3c2oc2ccccc23)cc1 ; c1ccc(-c2ccc3c(c2)oc2cccc(N(c4ccc5ccccc5c4)c4ccc(-c5ccccc5)c5oc6ccccc6c45)c23)cc1 ; c1ccc(-c2ccc(N(c3ccc(-c4cccc5ccccc45)cc3)c3cccc4oc5ccccc5c34)c3c2oc2ccccc23)cc1]

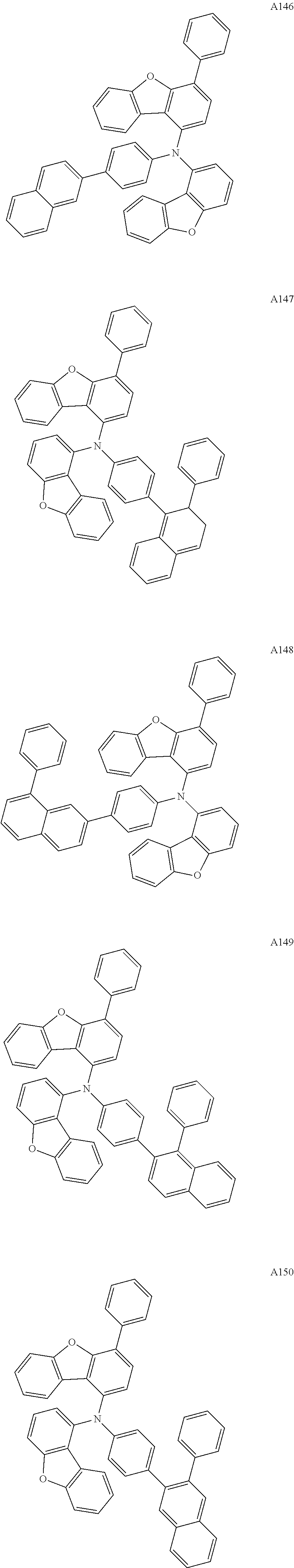 [SMILES: c1ccc(-c2ccc(N(c3ccc(-c4ccc5ccccc5c4)cc3)c3cccc4oc5ccccc5c34)c3c2oc2ccccc23)cc1 ; C1=c2ccccc2=C(c2ccc(N(c3cccc4oc5ccccc5c34)c3ccc(-c4ccccc4)c4oc5ccccc5c34)cc2)C(c2ccccc2)C1 ; c1ccc(-c2cccc3ccc(-c4ccc(N(c5cccc6oc7ccccc7c56)c5ccc(-c6ccccc6)c6oc7ccccc7c56)cc4)cc23)cc1 ; c1ccc(-c2c(-c3ccc(N(c4cccc5oc6ccccc6c45)c4ccc(-c5ccccc5)c5oc6ccccc6c45)cc3)ccc3ccccc23)cc1 ; c1ccc(-c2cc3ccccc3cc2-c2ccc(N(c3cccc4oc5ccccc5c34)c3ccc(-c4ccccc4)c4oc5ccccc5c34)cc2)cc1]

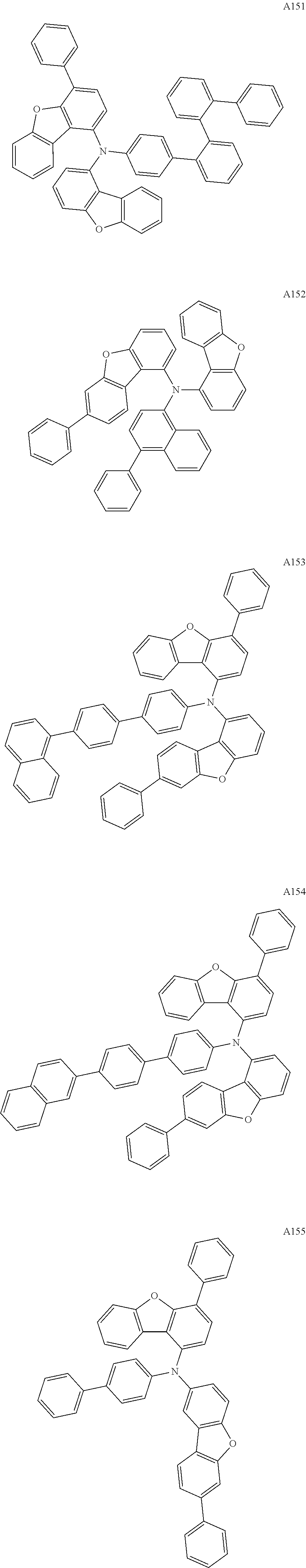 [SMILES: c1ccc(-c2ccccc2-c2ccccc2-c2ccc(N(c3cccc4oc5ccccc5c34)c3ccc(-c4ccccc4)c4oc5ccccc5c34)cc2)cc1 ; c1ccc(-c2ccc3c(c2)oc2cccc(N(c4ccc(-c5ccccc5)c5ccccc45)c4cccc5oc6ccccc6c45)c23)cc1 ; c1ccc(-c2ccc3c(c2)oc2cccc(N(c4ccc(-c5ccc(-c6cccc7ccccc67)cc5)cc4)c4ccc(-c5ccccc5)c5oc6ccccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4oc5cc(-c6ccccc6)ccc5c4c3)c3ccc(-c4ccccc4)c4oc5ccccc5c34)cc2)cc1]

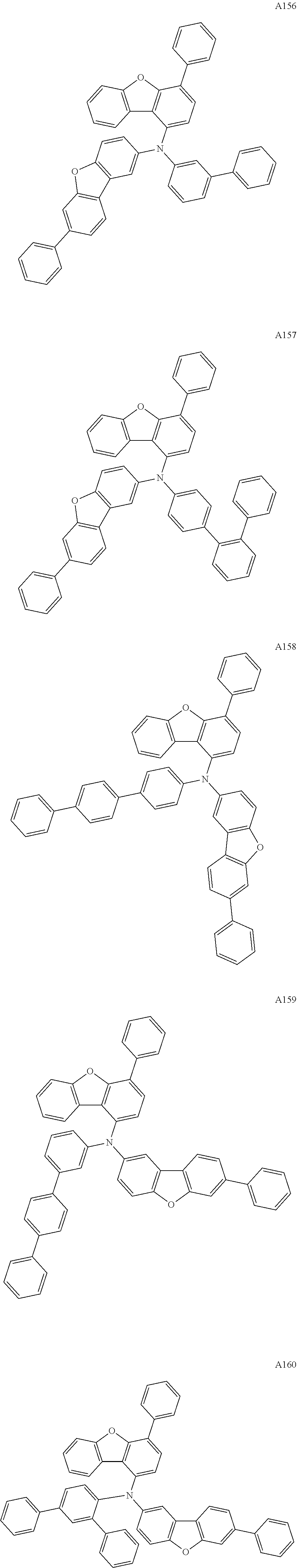 [SMILES: c1ccc(-c2cccc(N(c3ccc4oc5cc(-c6ccccc6)ccc5c4c3)c3ccc(-c4ccccc4)c4oc5ccccc5c34)c2)cc1 ; c1ccc(-c2ccc3c(c2)oc2ccc(N(c4ccc(-c5ccccc5-c5ccccc5)cc4)c4ccc(-c5ccccc5)c5oc6ccccc6c45)cc23)cc1 ; c1ccc(-c2ccc(-c3ccc(N(c4ccc5oc6cc(-c7ccccc7)ccc6c5c4)c4ccc(-c5ccccc5)c5oc6ccccc6c45)cc3)cc2)cc1 ; c1ccc(-c2ccc(-c3cccc(N(c4ccc5oc6cc(-c7ccccc7)ccc6c5c4)c4ccc(-c5ccccc5)c5oc6ccccc6c45)c3)cc2)cc1 ; c1ccc(-c2ccc(N(c3ccc4oc5cc(-c6ccccc6)ccc5c4c3)c3ccc(-c4ccccc4)c4oc5ccccc5c34)c(-c3ccccc3)c2)cc1]

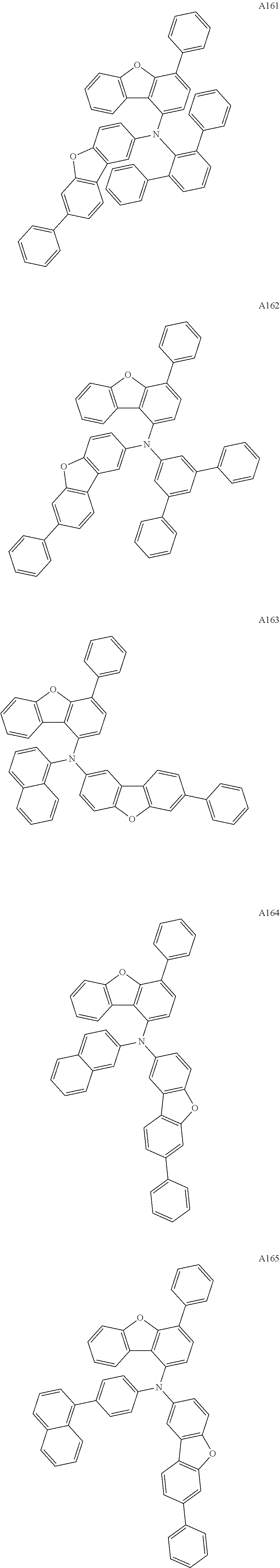 [SMILES: c1ccc(-c2ccc3c(c2)oc2ccc(N(c4c(-c5ccccc5)cccc4-c4ccccc4)c4ccc(-c5ccccc5)c5oc6ccccc6c45)cc23)cc1 ; c1ccc(-c2cc(-c3ccccc3)cc(N(c3ccc4oc5cc(-c6ccccc6)ccc5c4c3)c3ccc(-c4ccccc4)c4oc5ccccc5c34)c2)cc1 ; c1ccc(-c2ccc3c(c2)oc2ccc(N(c4cccc5ccccc45)c4ccc(-c5ccccc5)c5oc6ccccc6c45)cc23)cc1 ; c1ccc(-c2ccc3c(c2)oc2ccc(N(c4ccc5ccccc5c4)c4ccc(-c5ccccc5)c5oc6ccccc6c45)cc23)cc1 ; c1ccc(-c2ccc3c(c2)oc2ccc(N(c4ccc(-c5cccc6ccccc56)cc4)c4ccc(-c5ccccc5)c5oc6ccccc6c45)cc23)cc1]

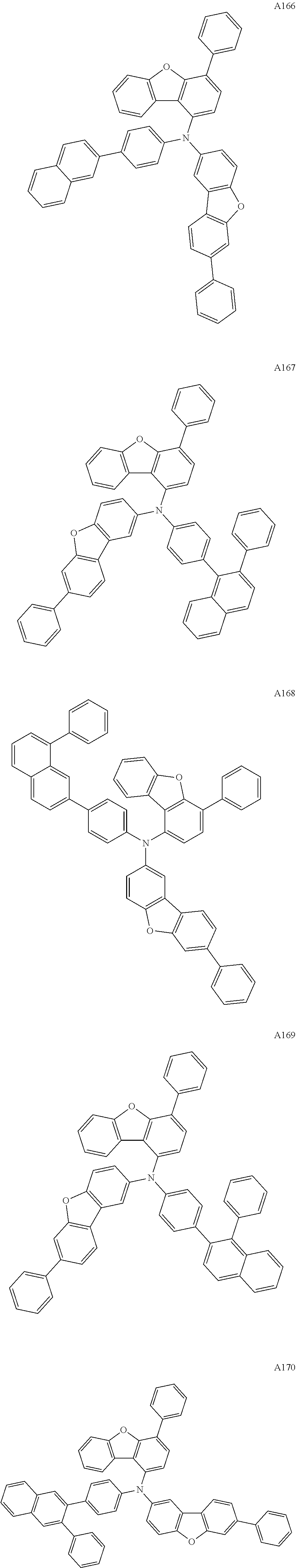

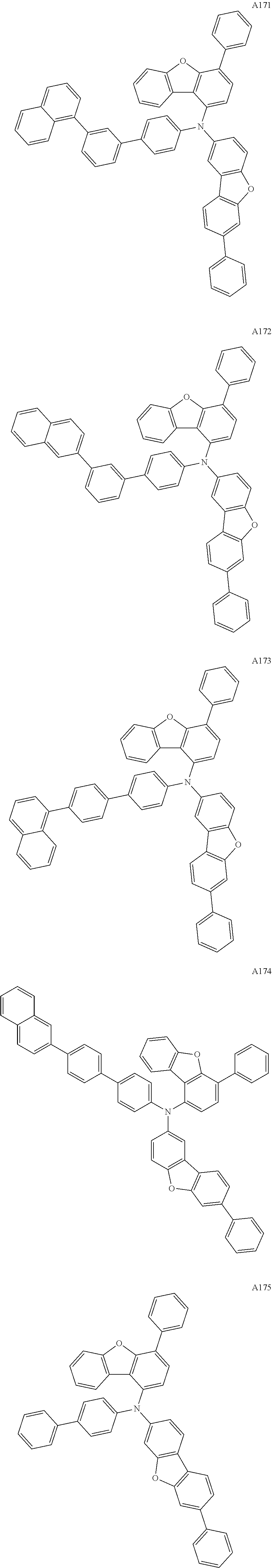

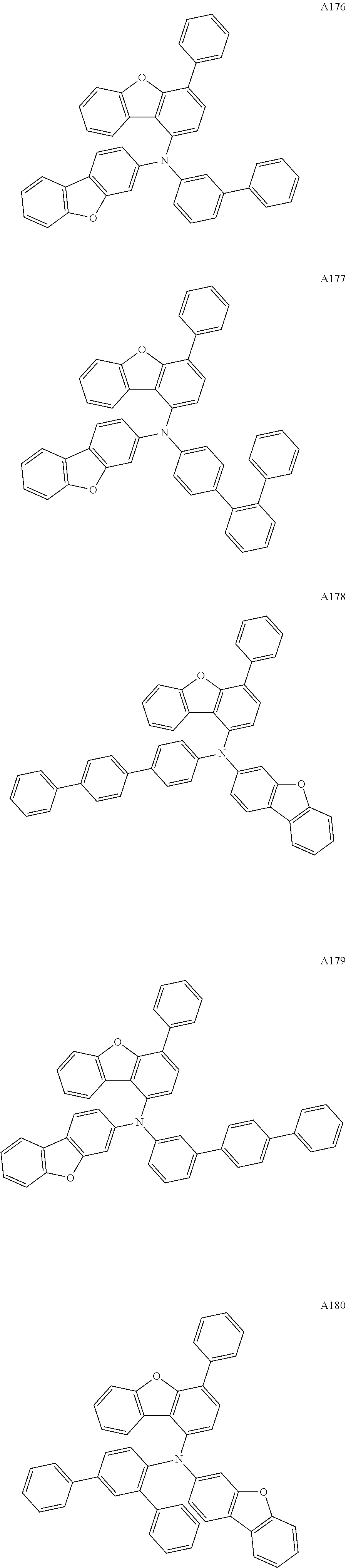 [SMILES: c1ccc(-c2cccc(N(c3ccc4c(c3)oc3ccccc34)c3ccc(-c4ccccc4)c4oc5ccccc5c34)c2)cc1 ; c1ccc(-c2ccccc2-c2ccc(N(c3ccc4c(c3)oc3ccccc34)c3ccc(-c4ccccc4)c4oc5ccccc5c34)cc2)cc1 ; c1ccc(-c2ccc(-c3ccc(N(c4ccc5c(c4)oc4ccccc45)c4ccc(-c5ccccc5)c5oc6ccccc6c45)cc3)cc2)cc1 ; c1ccc(-c2ccc(-c3cccc(N(c4ccc5c(c4)oc4ccccc45)c4ccc(-c5ccccc5)c5oc6ccccc6c45)c3)cc2)cc1 ; c1ccc(-c2ccc(N(c3ccc4c(c3)oc3ccccc34)c3ccc(-c4ccccc4)c4oc5ccccc5c34)c(-c3ccccc3)c2)cc1]

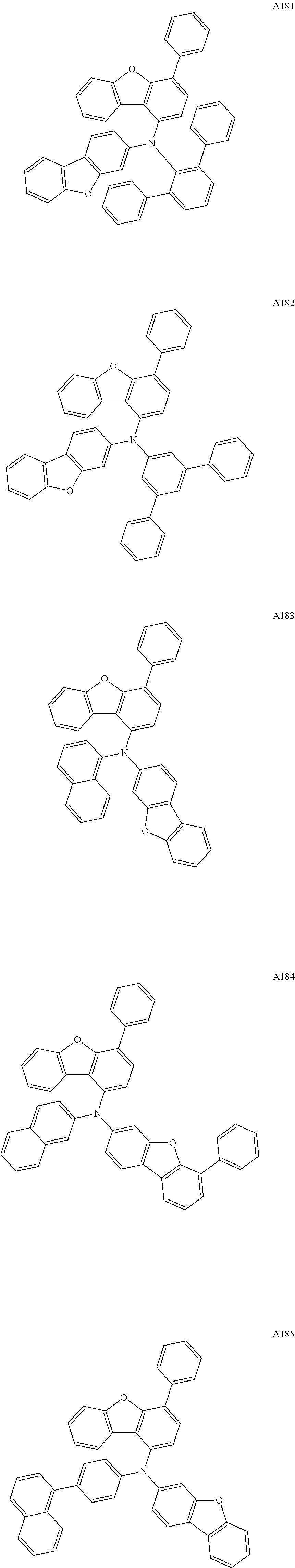 [SMILES: c1ccc(-c2cccc(-c3ccccc3)c2N(c2ccc3c(c2)oc2ccccc23)c2ccc(-c3ccccc3)c3oc4ccccc4c23)cc1 ; c1ccc(-c2cc(-c3ccccc3)cc(N(c3ccc4c(c3)oc3ccccc34)c3ccc(-c4ccccc4)c4oc5ccccc5c34)c2)cc1 ; c1ccc(-c2ccc(N(c3ccc4c(c3)oc3ccccc34)c3cccc4ccccc34)c3c2oc2ccccc23)cc1 ; c1ccc(-c2cccc3c2oc2cc(N(c4ccc5ccccc5c4)c4ccc(-c5ccccc5)c5oc6ccccc6c45)ccc23)cc1 ; c1ccc(-c2ccc(N(c3ccc(-c4cccc5ccccc45)cc3)c3ccc4c(c3)oc3ccccc34)c3c2oc2ccccc23)cc1]

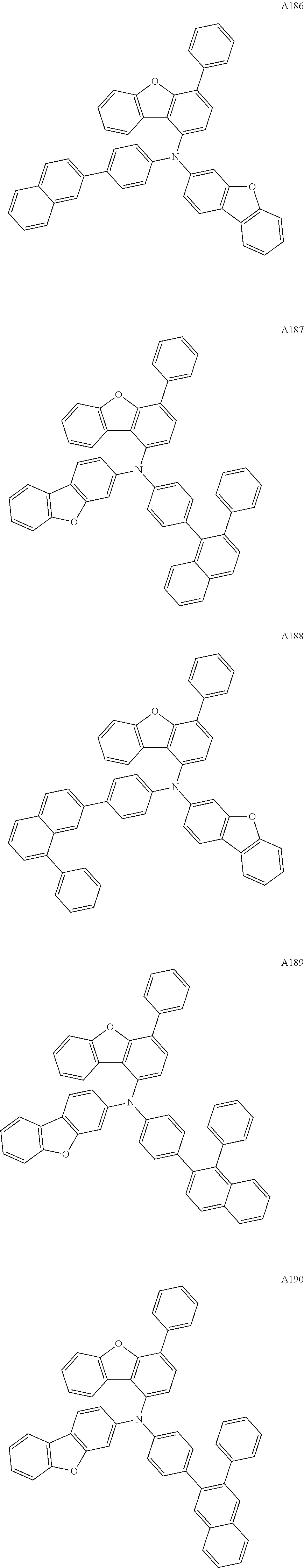 [SMILES: c1ccc(-c2ccc(N(c3ccc(-c4ccc5ccccc5c4)cc3)c3ccc4c(c3)oc3ccccc34)c3c2oc2ccccc23)cc1 ; c1ccc(-c2ccc3ccccc3c2-c2ccc(N(c3ccc4c(c3)oc3ccccc34)c3ccc(-c4ccccc4)c4oc5ccccc5c34)cc2)cc1 ; c1ccc(-c2cccc3ccc(-c4ccc(N(c5ccc6c(c5)oc5ccccc56)c5ccc(-c6ccccc6)c6oc7ccccc7c56)cc4)cc23)cc1 ; c1ccc(-c2c(-c3ccc(N(c4ccc5c(c4)oc4ccccc45)c4ccc(-c5ccccc5)c5oc6ccccc6c45)cc3)ccc3ccccc23)cc1 ; c1ccc(-c2cc3ccccc3cc2-c2ccc(N(c3ccc4c(c3)oc3ccccc34)c3ccc(-c4ccccc4)c4oc5ccccc5c34)cc2)cc1]

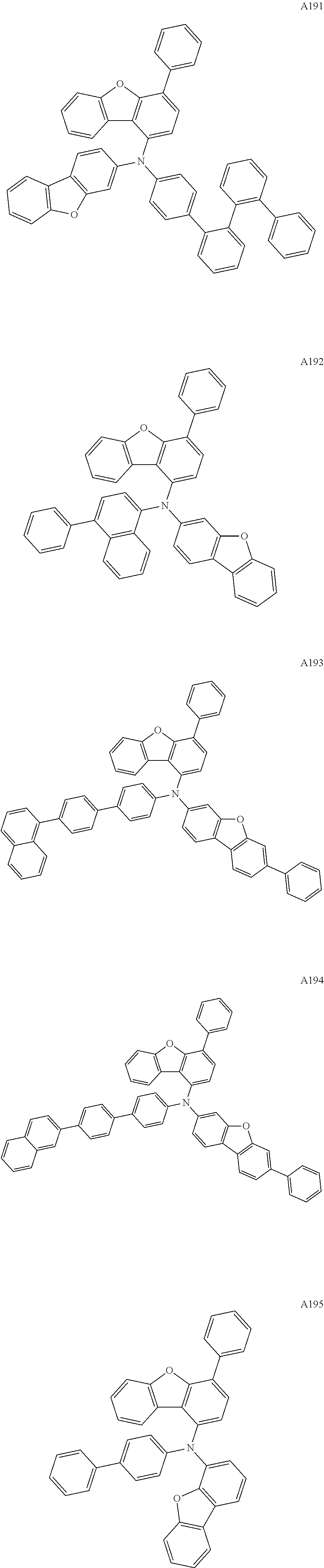 [SMILES: c1ccc(-c2ccccc2-c2ccccc2-c2ccc(N(c3ccc4c(c3)oc3ccccc34)c3ccc(-c4ccccc4)c4oc5ccccc5c34)cc2)cc1 ; c1ccc(-c2ccc(N(c3ccc4c(c3)oc3ccccc34)c3ccc(-c4ccccc4)c4oc5ccccc5c34)c3ccccc23)cc1 ; c1ccc(-c2ccc3c(c2)oc2cc(N(c4ccc(-c5ccc(-c6cccc7ccccc67)cc5)cc4)c4ccc(-c5ccccc5)c5oc6ccccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5oc6ccccc6c45)ccc23)cc1 ; c1ccc(-c2ccc(N(c3cccc4c3oc3ccccc34)c3ccc(-c4ccccc4)c4oc5ccccc5c34)cc2)cc1]

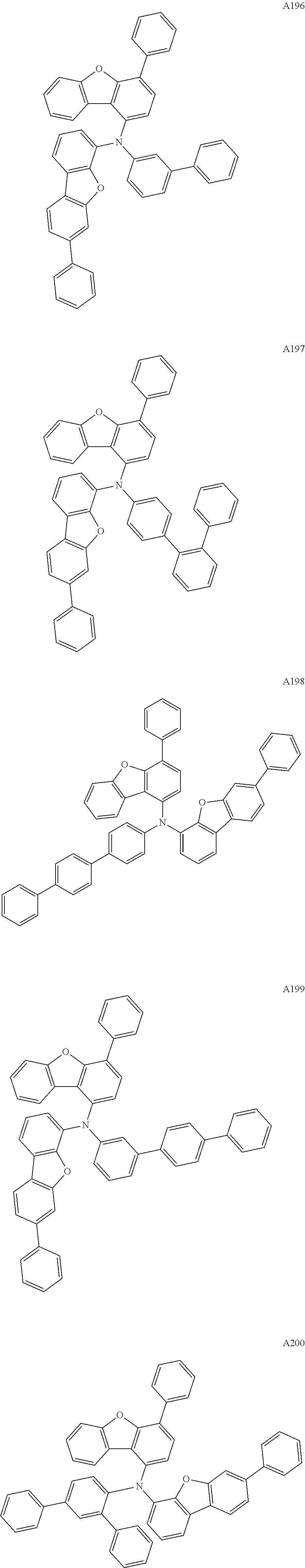 [SMILES: c1ccc(-c2cccc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4ccc(-c5ccccc5-c5ccccc5)cc4)c4ccc(-c5ccccc5)c5oc6ccccc6c45)cccc23)cc1 ; c1ccc(-c2ccc(-c3ccc(N(c4cccc5c4oc4cc(-c6ccccc6)ccc45)c4ccc(-c5ccccc5)c5oc6ccccc6c45)cc3)cc2)cc1 ; c1ccc(-c2ccc(-c3cccc(N(c4cccc5c4oc4cc(-c6ccccc6)ccc45)c4ccc(-c5ccccc5)c5oc6ccccc6c45)c3)cc2)cc1 ; c1ccc(-c2ccc(N(c3cccc4c3oc3cc(-c5ccccc5)ccc34)c3ccc(-c4ccccc4)c4oc5ccccc5c34)c(-c3ccccc3)c2)cc1]

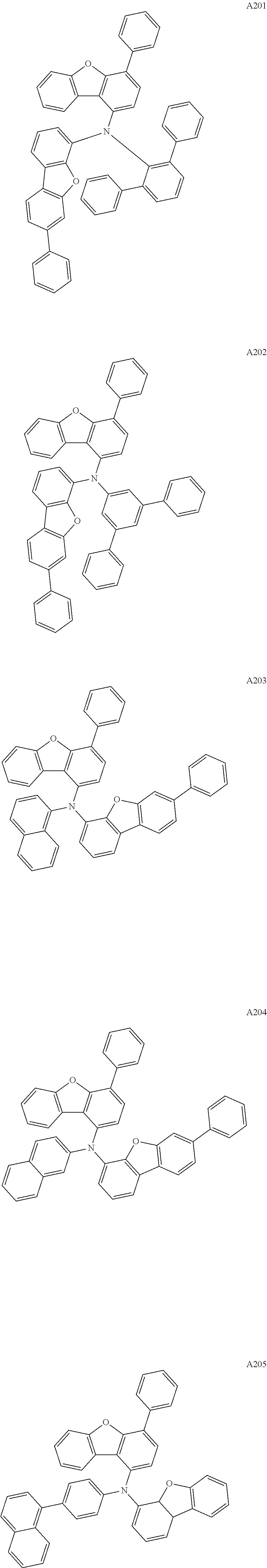 [SMILES: c1ccc(-c2ccc3c(c2)oc2c(N(c4c(-c5ccccc5)cccc4-c4ccccc4)c4ccc(-c5ccccc5)c5oc6ccccc6c45)cccc23)cc1 ; c1ccc(-c2cc(-c3ccccc3)cc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4cccc5ccccc45)c4ccc(-c5ccccc5)c5oc6ccccc6c45)cccc23)cc1 ; c1ccc(-c2ccc3c(c2)oc2c(N(c4ccc5ccccc5c4)c4ccc(-c5ccccc5)c5oc6ccccc6c45)cccc23)cc1 ; C1=CC2c3ccccc3OC2C(N(c2ccc(-c3cccc4ccccc34)cc2)c2ccc(-c3ccccc3)c3oc4ccccc4c23)=C1]

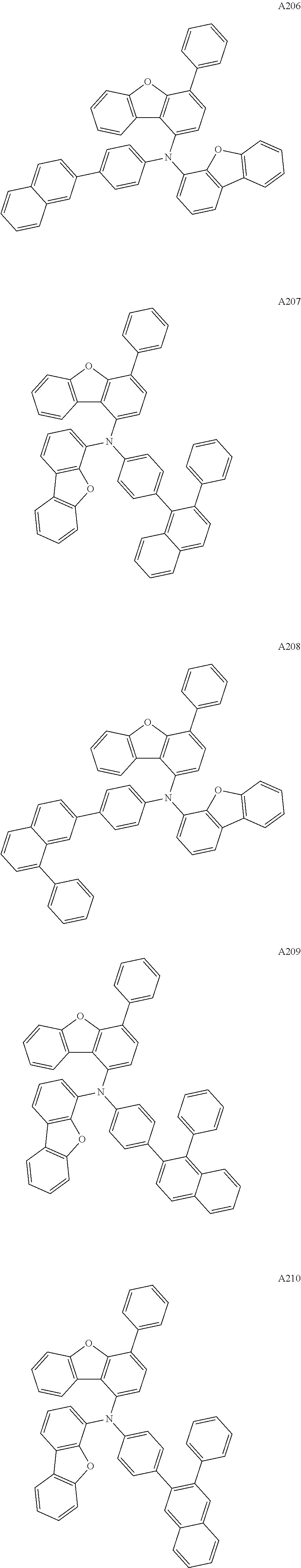 [SMILES: c1ccc(-c2ccc(N(c3ccc(-c4ccc5ccccc5c4)cc3)c3cccc4c3oc3ccccc34)c3c2oc2ccccc23)cc1 ; c1ccc(-c2ccc3ccccc3c2-c2ccc(N(c3cccc4c3oc3ccccc34)c3ccc(-c4ccccc4)c4oc5ccccc5c34)cc2)cc1 ; c1ccc(-c2cccc3ccc(-c4ccc(N(c5cccc6c5oc5ccccc56)c5ccc(-c6ccccc6)c6oc7ccccc7c56)cc4)cc23)cc1 ; c1ccc(-c2c(-c3ccc(N(c4cccc5c4oc4ccccc45)c4ccc(-c5ccccc5)c5oc6ccccc6c45)cc3)ccc3ccccc23)cc1 ; c1ccc(-c2cc3ccccc3cc2-c2ccc(N(c3cccc4c3oc3ccccc34)c3ccc(-c4ccccc4)c4oc5ccccc5c34)cc2)cc1]

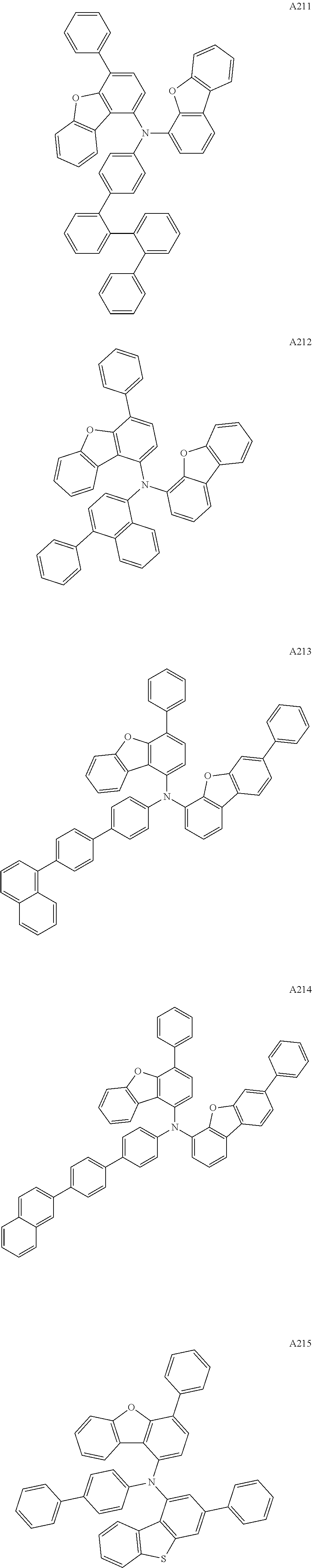 [SMILES: c1ccc(-c2ccccc2-c2ccccc2-c2ccc(N(c3cccc4c3oc3ccccc34)c3ccc(-c4ccccc4)c4oc5ccccc5c34)cc2)cc1 ; c1ccc(-c2ccc(N(c3cccc4c3oc3ccccc34)c3ccc(-c4ccccc4)c4oc5ccccc5c34)c3ccccc23)cc1 ; c1ccc(-c2ccc3c(c2)oc2c(N(c4ccc(-c5ccc(-c6cccc7ccccc67)cc5)cc4)c4ccc(-c5ccccc5)c5oc6ccccc6c45)cccc23)cc1 ; c1ccc(-c2ccc3c(c2)oc2c(N(c4ccc(-c5ccc(-c6ccc7ccccc7c6)cc5)cc4)c4ccc(-c5ccccc5)c5oc6ccccc6c45)cccc23)cc1 ; c1ccc(-c2ccc(N(c3cc(-c4ccccc4)cc4sc5ccccc5c34)c3ccc(-c4ccccc4)c4oc5ccccc5c34)cc2)cc1]

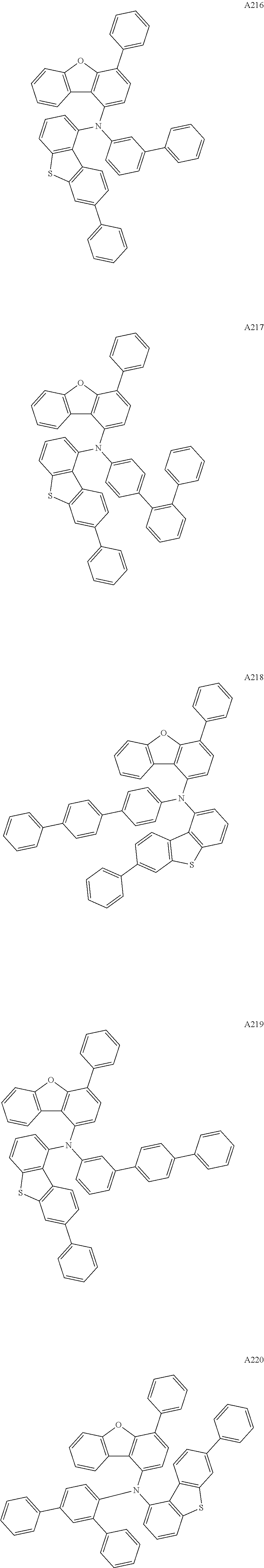 [SMILES: c1ccc(-c2cccc(N(c3cccc4sc5cc(-c6ccccc6)ccc5c34)c3ccc(-c4ccccc4)c4oc5ccccc5c34)c2)cc1 ; c1ccc(-c2ccc3c(c2)sc2cccc(N(c4ccc(-c5ccccc5-c5ccccc5)cc4)c4ccc(-c5ccccc5)c5oc6ccccc6c45)c23)cc1 ; c1ccc(-c2ccc(-c3ccc(N(c4cccc5sc6cc(-c7ccccc7)ccc6c45)c4ccc(-c5ccccc5)c5oc6ccccc6c45)cc3)cc2)cc1 ; c1ccc(-c2ccc(-c3cccc(N(c4cccc5sc6cc(-c7ccccc7)ccc6c45)c4ccc(-c5ccccc5)c5oc6ccccc6c45)c3)cc2)cc1 ; c1ccc(-c2ccc(N(c3cccc4sc5cc(-c6ccccc6)ccc5c34)c3ccc(-c4ccccc4)c4oc5ccccc5c34)c(-c3ccccc3)c2)cc1]

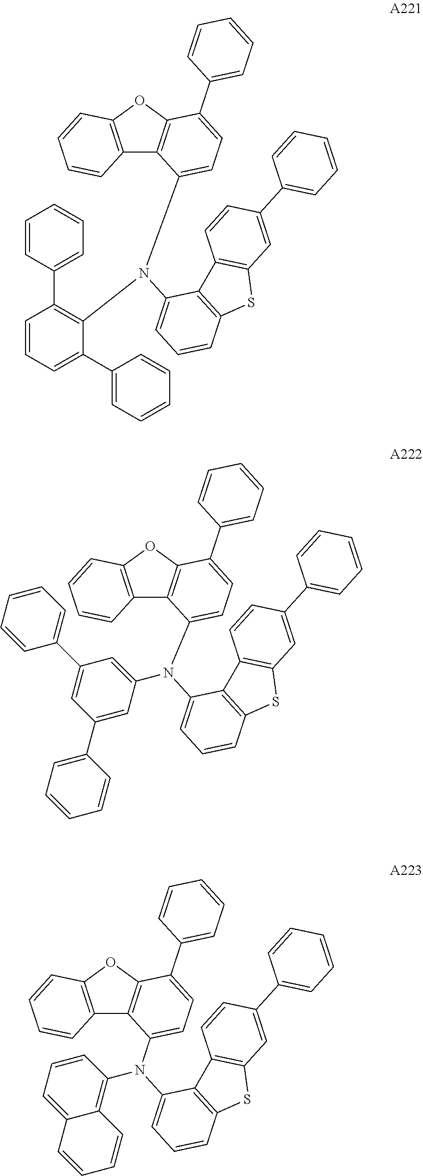 [SMILES: c1ccc(-c2ccc3c(c2)sc2cccc(N(c4c(-c5ccccc5)cccc4-c4ccccc4)c4ccc(-c5ccccc5)c5oc6ccccc6c45)c23)cc1 ; c1ccc(-c2cc(-c3ccccc3)cc(N(c3cccc4sc5cc(-c6ccccc6)ccc5c34)c3ccc(-c4ccccc4)c4oc5ccccc5c34)c2)cc1 ; c1ccc(-c2ccc3c(c2)sc2cccc(N(c4cccc5ccccc45)c4ccc(-c5ccccc5)c5oc6ccccc6c45)c23)cc1]

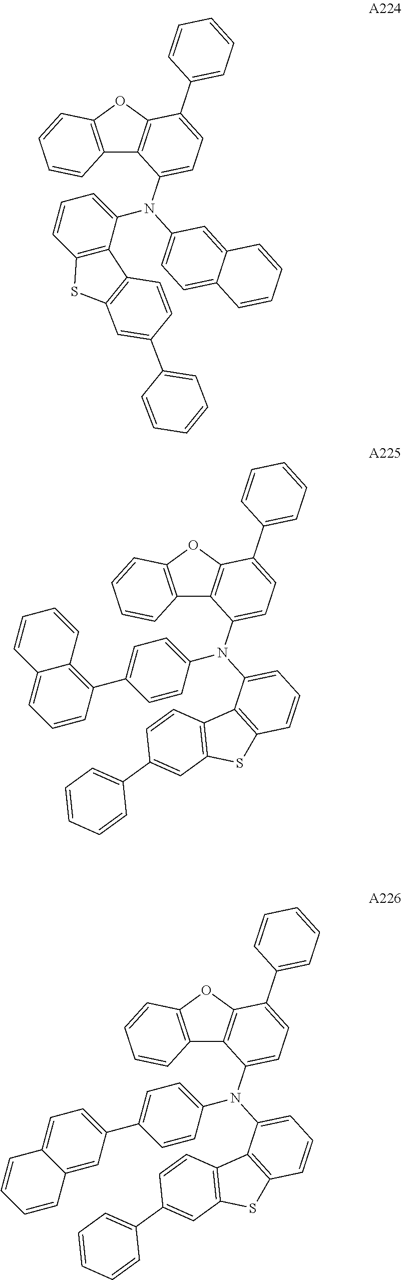 [SMILES: c1ccc(-c2ccc3c(c2)sc2cccc(N(c4ccc5ccccc5c4)c4ccc(-c5ccccc5)c5oc6ccccc6c45)c23)cc1 ; c1ccc(-c2ccc3c(c2)sc2cccc(N(c4ccc(-c5cccc6ccccc56)cc4)c4ccc(-c5ccccc5)c5oc6ccccc6c45)c23)cc1 ; c1ccc(-c2ccc3c(c2)sc2cccc(N(c4ccc(-c5ccc6ccccc6c5)cc4)c4ccc(-c5ccccc5)c5oc6ccccc6c45)c23)cc1]

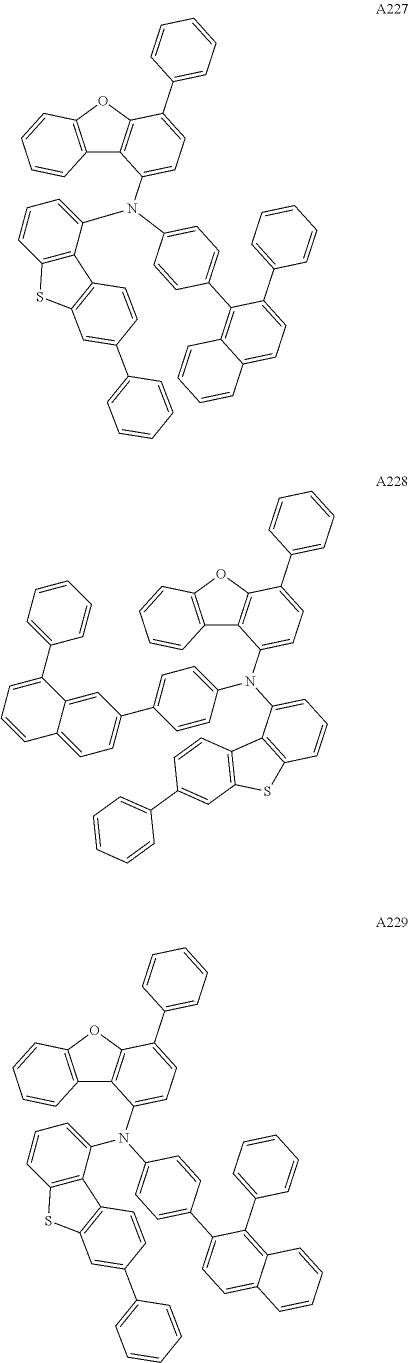 [SMILES: c1ccc(-c2ccc3c(c2)sc2cccc(N(c4ccc(-c5c(-c6ccccc6)ccc6ccccc56)cc4)c4ccc(-c5ccccc5)c5oc6ccccc6c45)c23)cc1 ; c1ccc(-c2ccc3c(c2)sc2cccc(N(c4ccc(-c5ccc6cccc(-c7ccccc7)c6c5)cc4)c4ccc(-c5ccccc5)c5oc6ccccc6c45)c23)cc1 ; c1ccc(-c2ccc3c(c2)sc2cccc(N(c4ccc(-c5ccc6ccccc6c5-c5ccccc5)cc4)c4ccc(-c5ccccc5)c5oc6ccccc6c45)c23)cc1]

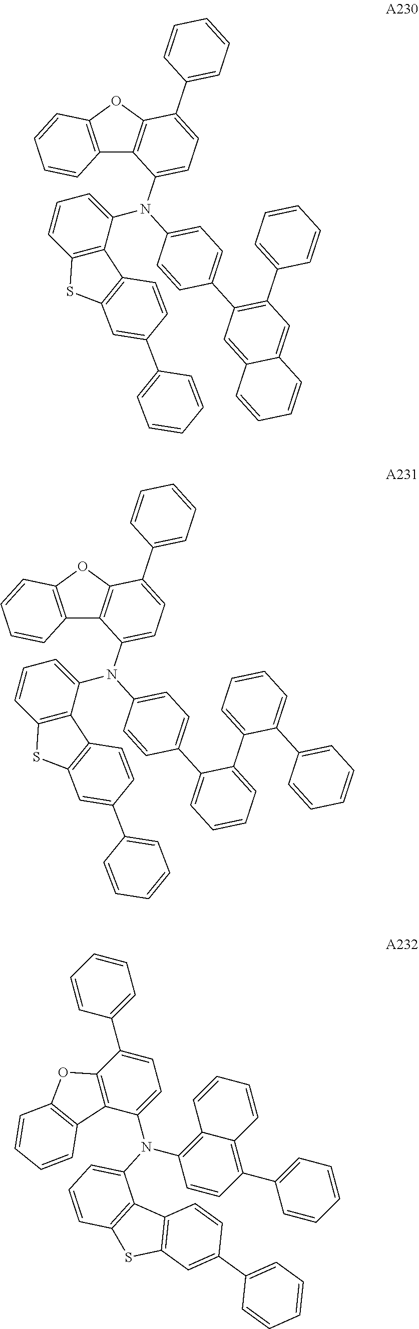 [SMILES: c1ccc(-c2ccc3c(c2)sc2cccc(N(c4ccc(-c5cc6ccccc6cc5-c5ccccc5)cc4)c4ccc(-c5ccccc5)c5oc6ccccc6c45)c23)cc1 ; c1ccc(-c2ccc3c(c2)sc2cccc(N(c4ccc(-c5ccccc5-c5ccccc5-c5ccccc5)cc4)c4ccc(-c5ccccc5)c5oc6ccccc6c45)c23)cc1 ; c1ccc(-c2ccc3c(c2)sc2cccc(N(c4ccc(-c5ccccc5)c5ccccc45)c4ccc(-c5ccccc5)c5oc6ccccc6c45)c23)cc1]

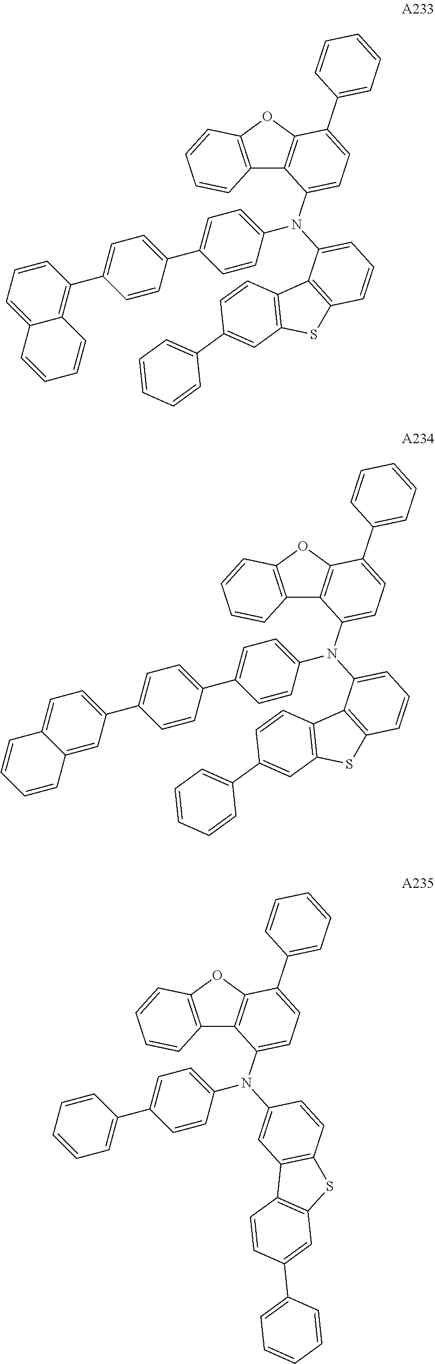 [SMILES: c1ccc(-c2ccc3c(c2)sc2cccc(N(c4ccc(-c5ccc(-c6cccc7ccccc67)cc5)cc4)c4ccc(-c5ccccc5)c5oc6ccccc6c45)c23)cc1 ; c1ccc(-c2ccc3c(c2)sc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4sc5cc(-c6ccccc6)ccc5c4c3)c3ccc(-c4ccccc4)c4oc5ccccc5c34)cc2)cc1]

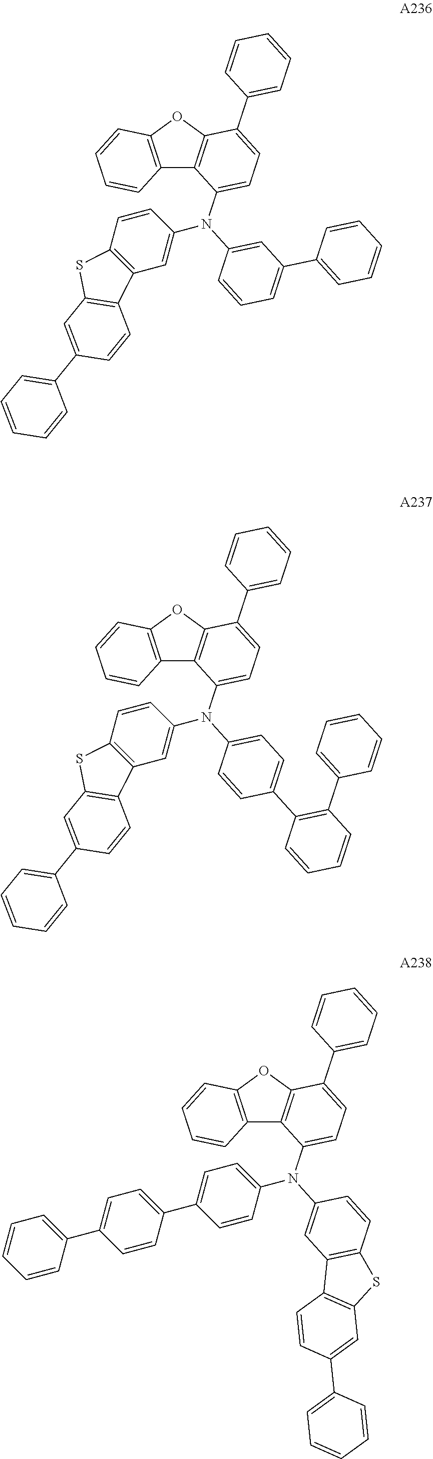 [SMILES: c1ccc(-c2cccc(N(c3ccc4sc5cc(-c6ccccc6)ccc5c4c3)c3ccc(-c4ccccc4)c4oc5ccccc5c34)c2)cc1 ; c1ccc(-c2ccc3c(c2)sc2ccc(N(c4ccc(-c5ccccc5-c5ccccc5)cc4)c4ccc(-c5ccccc5)c5oc6ccccc6c45)cc23)cc1 ; c1ccc(-c2ccc(-c3ccc(N(c4ccc5sc6cc(-c7ccccc7)ccc6c5c4)c4ccc(-c5ccccc5)c5oc6ccccc6c45)cc3)cc2)cc1]

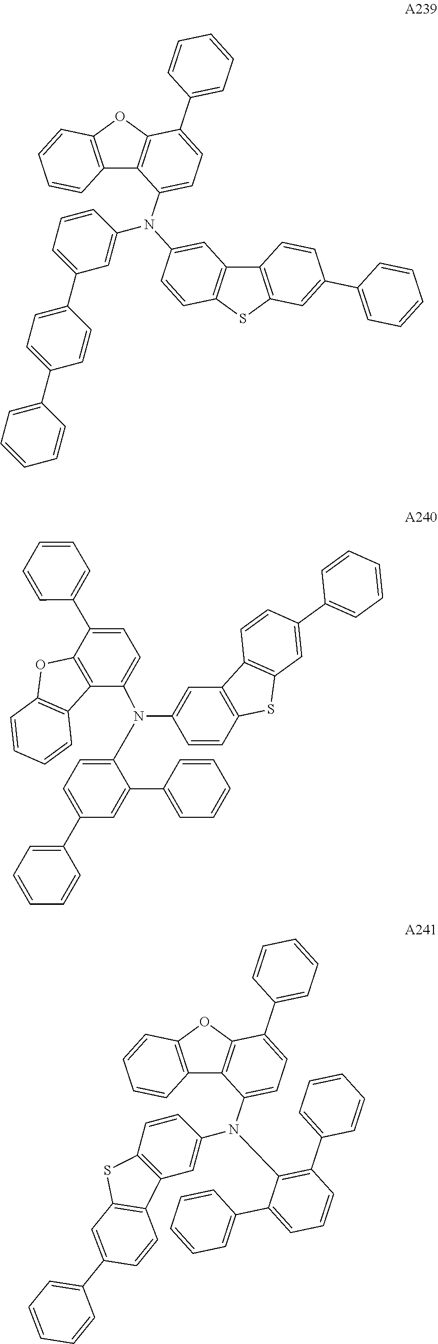 [SMILES: c1ccc(-c2ccc(-c3cccc(N(c4ccc5sc6cc(-c7ccccc7)ccc6c5c4)c4ccc(-c5ccccc5)c5oc6ccccc6c45)c3)cc2)cc1 ; c1ccc(-c2ccc(N(c3ccc4sc5cc(-c6ccccc6)ccc5c4c3)c3ccc(-c4ccccc4)c4oc5ccccc5c34)c(-c3ccccc3)c2)cc1 ; c1ccc(-c2ccc3c(c2)sc2ccc(N(c4c(-c5ccccc5)cccc4-c4ccccc4)c4ccc(-c5ccccc5)c5oc6ccccc6c45)cc23)cc1]

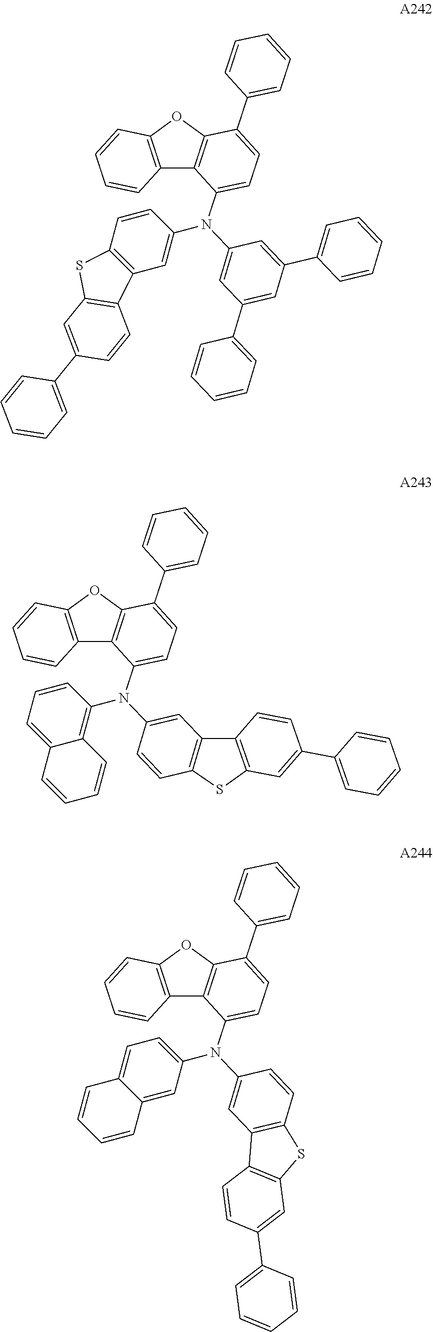 [SMILES: c1ccc(-c2cc(-c3ccccc3)cc(N(c3ccc4sc5cc(-c6ccccc6)ccc5c4c3)c3ccc(-c4ccccc4)c4oc5ccccc5c34)c2)cc1 ; c1ccc(-c2ccc3c(c2)sc2ccc(N(c4cccc5ccccc45)c4ccc(-c5ccccc5)c5oc6ccccc6c45)cc23)cc1 ; c1ccc(-c2ccc3c(c2)sc2ccc(N(c4ccc5ccccc5c4)c4ccc(-c5ccccc5)c5oc6ccccc6c45)cc23)cc1]

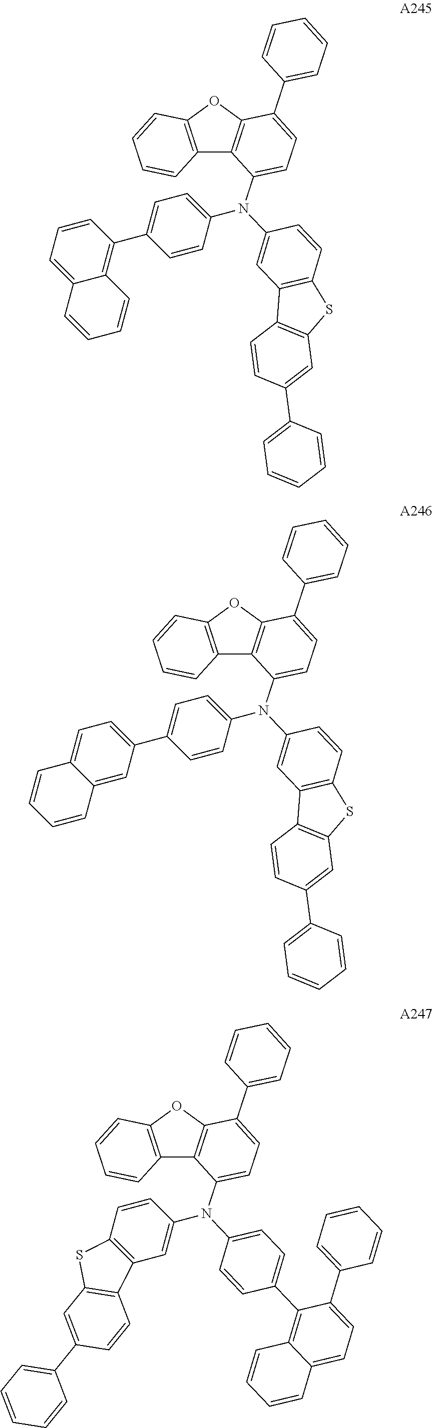

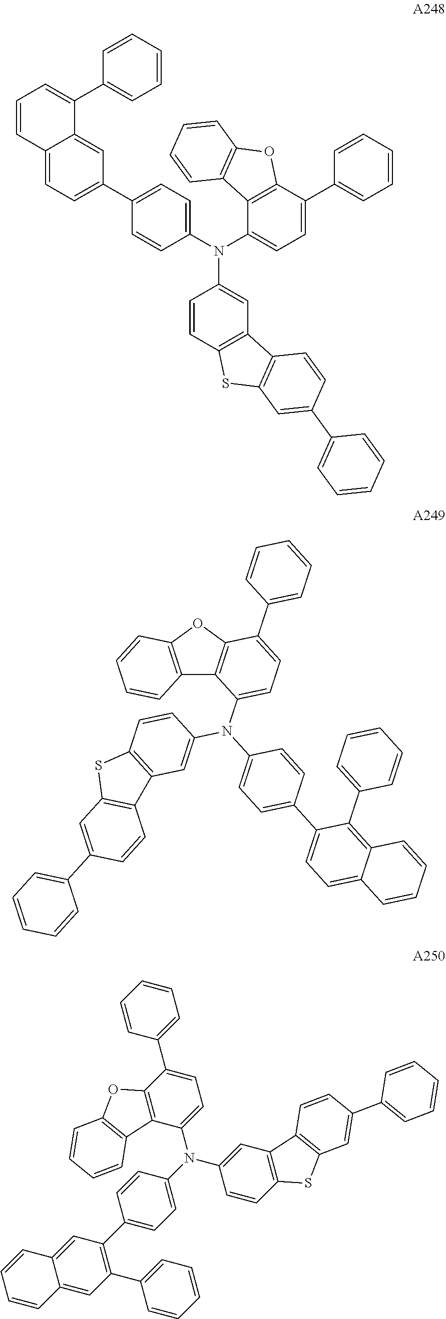 [SMILES: c1ccc(-c2ccc3c(c2)sc2ccc(N(c4ccc(-c5ccc6cccc(-c7ccccc7)c6c5)cc4)c4ccc(-c5ccccc5)c5oc6ccccc6c45)cc23)cc1 ; c1ccc(-c2ccc3c(c2)sc2ccc(N(c4ccc(-c5ccc6ccccc6c5-c5ccccc5)cc4)c4ccc(-c5ccccc5)c5oc6ccccc6c45)cc23)cc1 ; c1ccc(-c2ccc3c(c2)sc2ccc(N(c4ccc(-c5cc6ccccc6cc5-c5ccccc5)cc4)c4ccc(-c5ccccc5)c5oc6ccccc6c45)cc23)cc1]

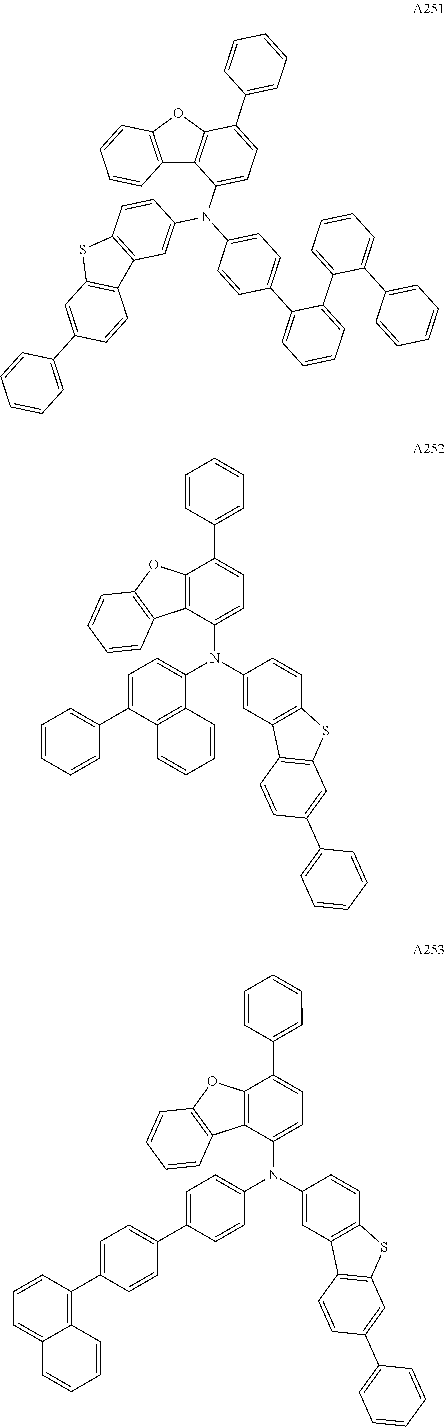 [SMILES: c1ccc(-c2ccc3c(c2)sc2ccc(N(c4ccc(-c5ccccc5-c5ccccc5-c5ccccc5)cc4)c4ccc(-c5ccccc5)c5oc6ccccc6c45)cc23)cc1 ; c1ccc(-c2ccc3c(c2)sc2ccc(N(c4ccc(-c5ccccc5)c5ccccc45)c4ccc(-c5ccccc5)c5oc6ccccc6c45)cc23)cc1 ; c1ccc(-c2ccc3c(c2)sc2ccc(N(c4ccc(-c5ccc(-c6cccc7ccccc67)cc5)cc4)c4ccc(-c5ccccc5)c5oc6ccccc6c45)cc23)cc1]

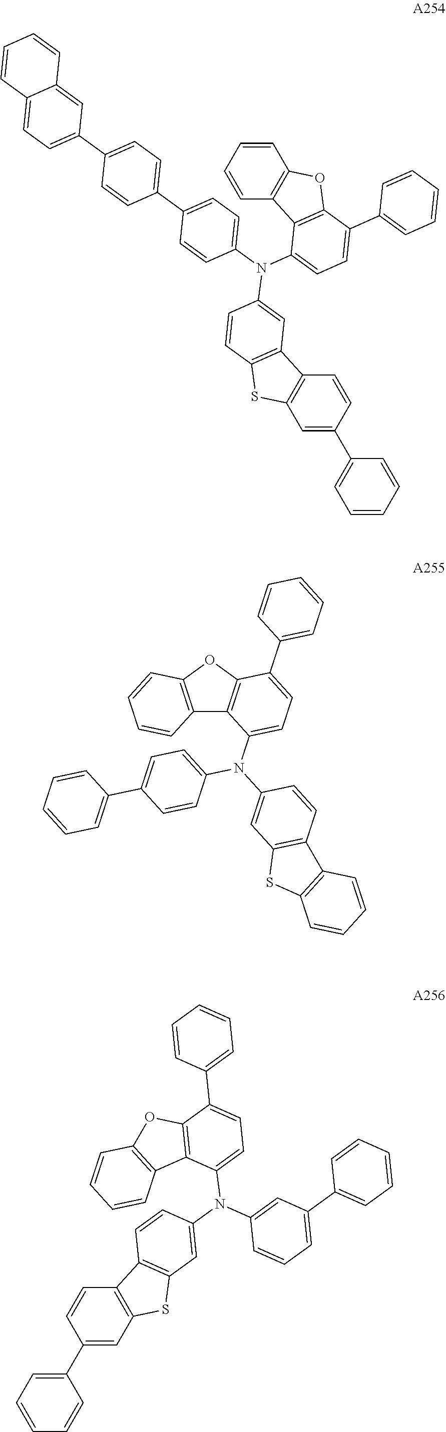 [SMILES: c1ccc(-c2ccc3c(c2)sc2ccc(N(c4ccc(-c5ccc(-c6ccc7ccccc7c6)cc5)cc4)c4ccc(-c5ccccc5)c5oc6ccccc6c45)cc23)cc1 ; c1ccc(-c2ccc(N(c3ccc4c(c3)sc3ccccc34)c3ccc(-c4ccccc4)c4oc5ccccc5c34)cc2)cc1 ; c1ccc(-c2cccc(N(c3ccc4c(c3)sc3cc(-c5ccccc5)ccc34)c3ccc(-c4ccccc4)c4oc5ccccc5c34)c2)cc1]

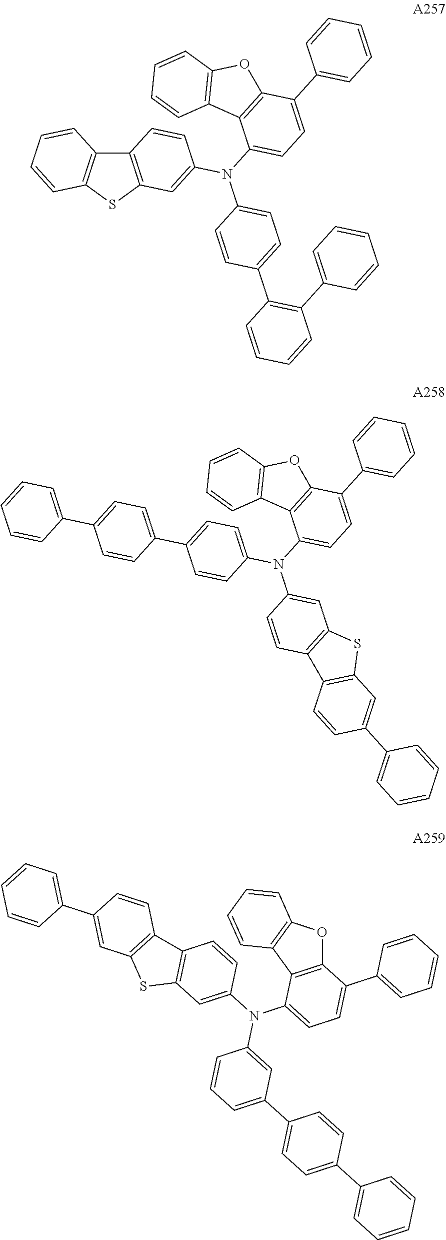 [SMILES: c1ccc(-c2ccccc2-c2ccc(N(c3ccc4c(c3)sc3ccccc34)c3ccc(-c4ccccc4)c4oc5ccccc5c34)cc2)cc1 ; c1ccc(-c2ccc(-c3ccc(N(c4ccc5c(c4)sc4cc(-c6ccccc6)ccc45)c4ccc(-c5ccccc5)c5oc6ccccc6c45)cc3)cc2)cc1 ; c1ccc(-c2ccc(-c3cccc(N(c4ccc5c(c4)sc4cc(-c6ccccc6)ccc45)c4ccc(-c5ccccc5)c5oc6ccccc6c45)c3)cc2)cc1]

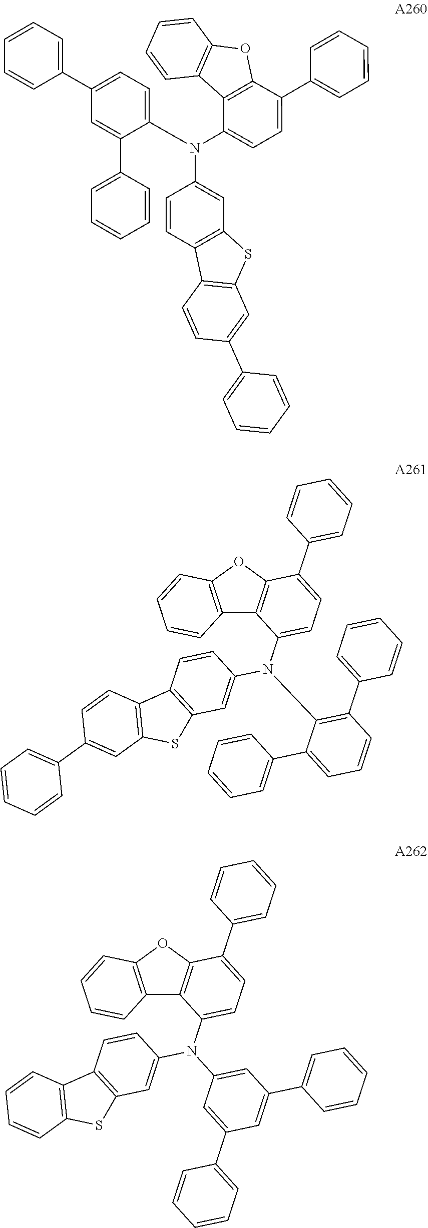 [SMILES: c1ccc(-c2ccc(N(c3ccc4c(c3)sc3cc(-c5ccccc5)ccc34)c3ccc(-c4ccccc4)c4oc5ccccc5c34)c(-c3ccccc3)c2)cc1 ; c1ccc(-c2ccc3c(c2)sc2cc(N(c4c(-c5ccccc5)cccc4-c4ccccc4)c4ccc(-c5ccccc5)c5oc6ccccc6c45)ccc23)cc1 ; c1ccc(-c2cc(-c3ccccc3)cc(N(c3ccc4c(c3)sc3ccccc34)c3ccc(-c4ccccc4)c4oc5ccccc5c34)c2)cc1]

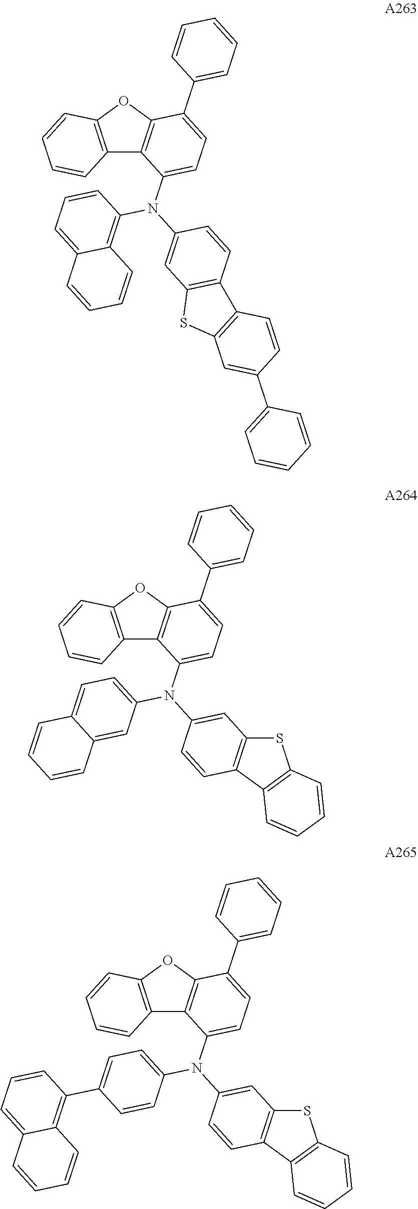 [SMILES: c1ccc(-c2ccc3c(c2)sc2cc(N(c4cccc5ccccc45)c4ccc(-c5ccccc5)c5oc6ccccc6c45)ccc23)cc1 ; c1ccc(-c2ccc(N(c3ccc4ccccc4c3)c3ccc4c(c3)sc3ccccc34)c3c2oc2ccccc23)cc1 ; c1ccc(-c2ccc(N(c3ccc(-c4cccc5ccccc45)cc3)c3ccc4c(c3)sc3ccccc34)c3c2oc2ccccc23)cc1]

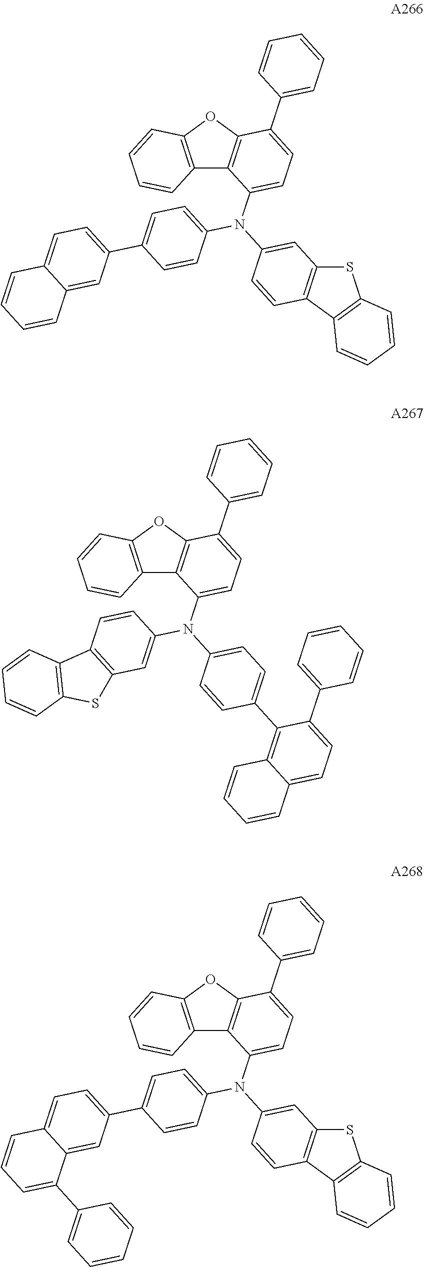 [SMILES: c1ccc(-c2ccc(N(c3ccc(-c4ccc5ccccc5c4)cc3)c3ccc4c(c3)sc3ccccc34)c3c2oc2ccccc23)cc1 ; c1ccc(-c2ccc3ccccc3c2-c2ccc(N(c3ccc4c(c3)sc3ccccc34)c3ccc(-c4ccccc4)c4oc5ccccc5c34)cc2)cc1 ; c1ccc(-c2cccc3ccc(-c4ccc(N(c5ccc6c(c5)sc5ccccc56)c5ccc(-c6ccccc6)c6oc7ccccc7c56)cc4)cc23)cc1]

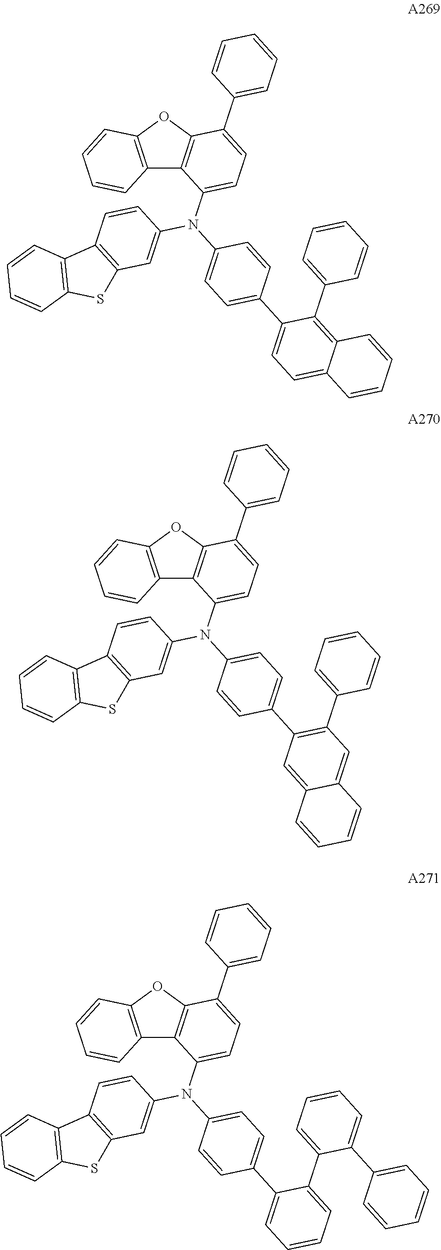 [SMILES: c1ccc(-c2c(-c3ccc(N(c4ccc5c(c4)sc4ccccc45)c4ccc(-c5ccccc5)c5oc6ccccc6c45)cc3)ccc3ccccc23)cc1 ; c1ccc(-c2cc3ccccc3cc2-c2ccc(N(c3ccc4c(c3)sc3ccccc34)c3ccc(-c4ccccc4)c4oc5ccccc5c34)cc2)cc1 ; c1ccc(-c2ccccc2-c2ccccc2-c2ccc(N(c3ccc4c(c3)sc3ccccc34)c3ccc(-c4ccccc4)c4oc5ccccc5c34)cc2)cc1]

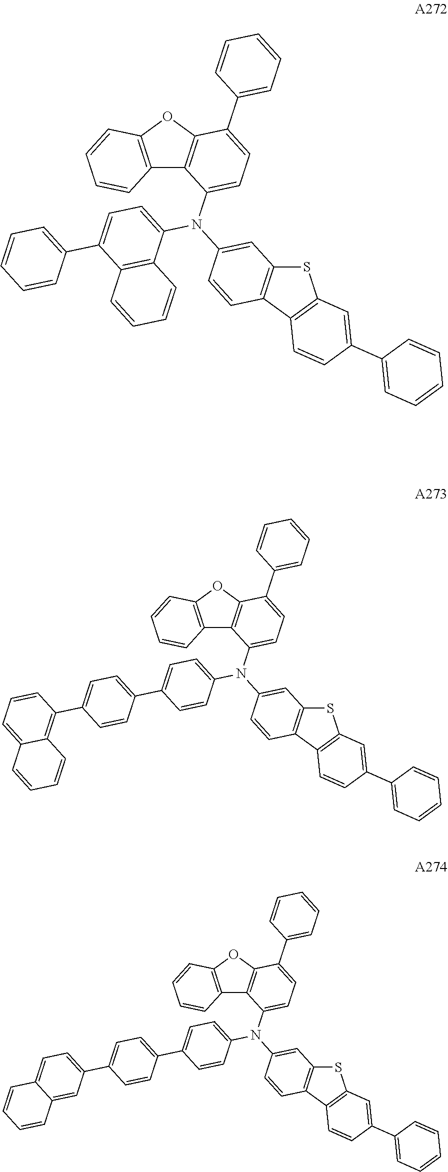 [SMILES: c1ccc(-c2ccc3c(c2)sc2cc(N(c4ccc(-c5ccccc5)c5ccccc45)c4ccc(-c5ccccc5)c5oc6ccccc6c45)ccc23)cc1 ; c1ccc(-c2ccc3c(c2)sc2cc(N(c4ccc(-c5ccc(-c6cccc7ccccc67)cc5)cc4)c4ccc(-c5ccccc5)c5oc6ccccc6c45)ccc23)cc1 ; c1ccc(-c2ccc3c(c2)sc2cc(N(c4ccc(-c5ccc(-c6ccc7ccccc7c6)cc5)cc4)c4ccc(-c5ccccc5)c5oc6ccccc6c45)ccc23)cc1]

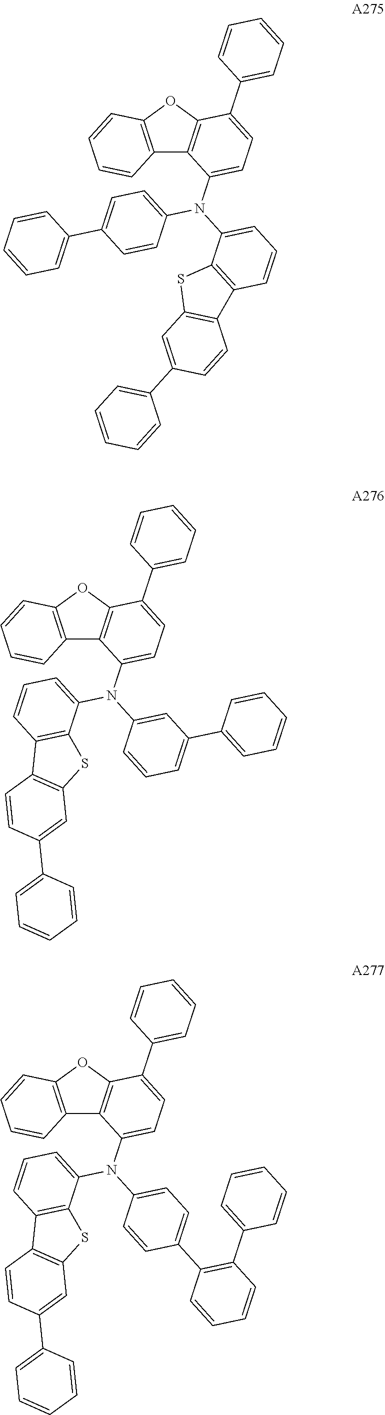 [SMILES: c1ccc(-c2ccc(N(c3cccc4c3sc3cc(-c5ccccc5)ccc34)c3ccc(-c4ccccc4)c4oc5ccccc5c34)cc2)cc1 ; c1ccc(-c2cccc(N(c3cccc4c3sc3cc(-c5ccccc5)ccc34)c3ccc(-c4ccccc4)c4oc5ccccc5c34)c2)cc1 ; c1ccc(-c2ccc3c(c2)sc2c(N(c4ccc(-c5ccccc5-c5ccccc5)cc4)c4ccc(-c5ccccc5)c5oc6ccccc6c45)cccc23)cc1]

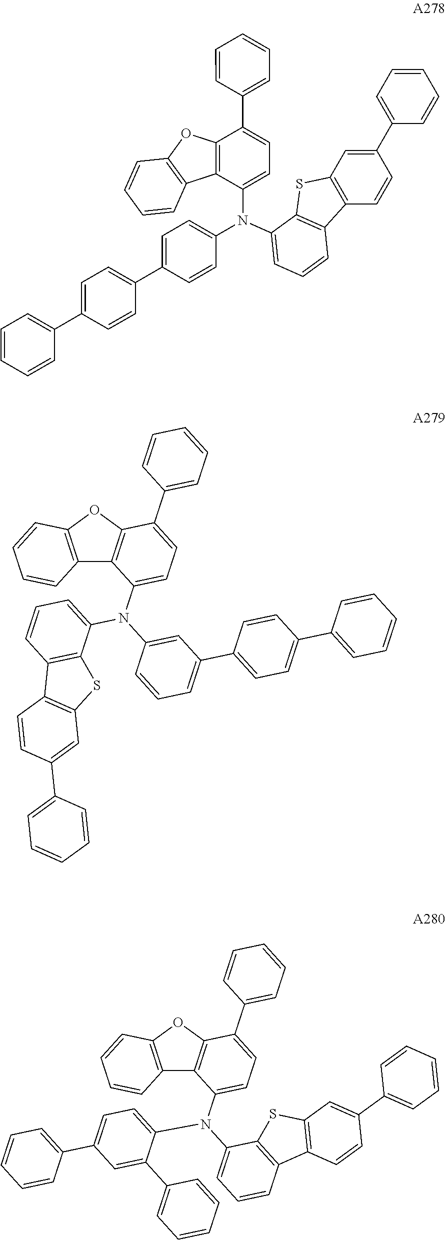 [SMILES: c1ccc(-c2ccc(-c3ccc(N(c4cccc5c4sc4cc(-c6ccccc6)ccc45)c4ccc(-c5ccccc5)c5oc6ccccc6c45)cc3)cc2)cc1 ; c1ccc(-c2ccc(-c3cccc(N(c4cccc5c4sc4cc(-c6ccccc6)ccc45)c4ccc(-c5ccccc5)c5oc6ccccc6c45)c3)cc2)cc1 ; c1ccc(-c2ccc(N(c3cccc4c3sc3cc(-c5ccccc5)ccc34)c3ccc(-c4ccccc4)c4oc5ccccc5c34)c(-c3ccccc3)c2)cc1]

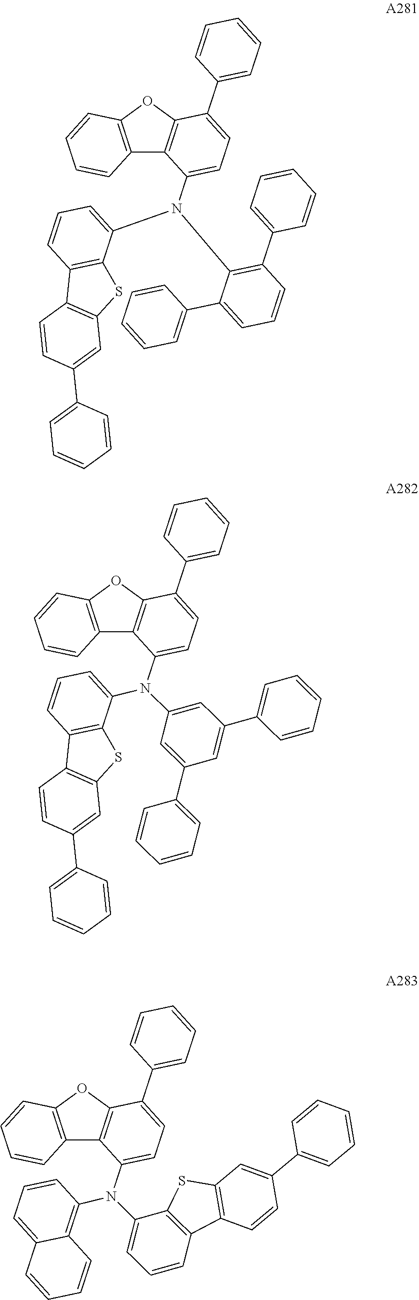 [SMILES: c1ccc(-c2ccc3c(c2)sc2c(N(c4c(-c5ccccc5)cccc4-c4ccccc4)c4ccc(-c5ccccc5)c5oc6ccccc6c45)cccc23)cc1 ; c1ccc(-c2cc(-c3ccccc3)cc(N(c3cccc4c3sc3cc(-c5ccccc5)ccc34)c3ccc(-c4ccccc4)c4oc5ccccc5c34)c2)cc1 ; c1ccc(-c2ccc3c(c2)sc2c(N(c4cccc5ccccc45)c4ccc(-c5ccccc5)c5oc6ccccc6c45)cccc23)cc1]

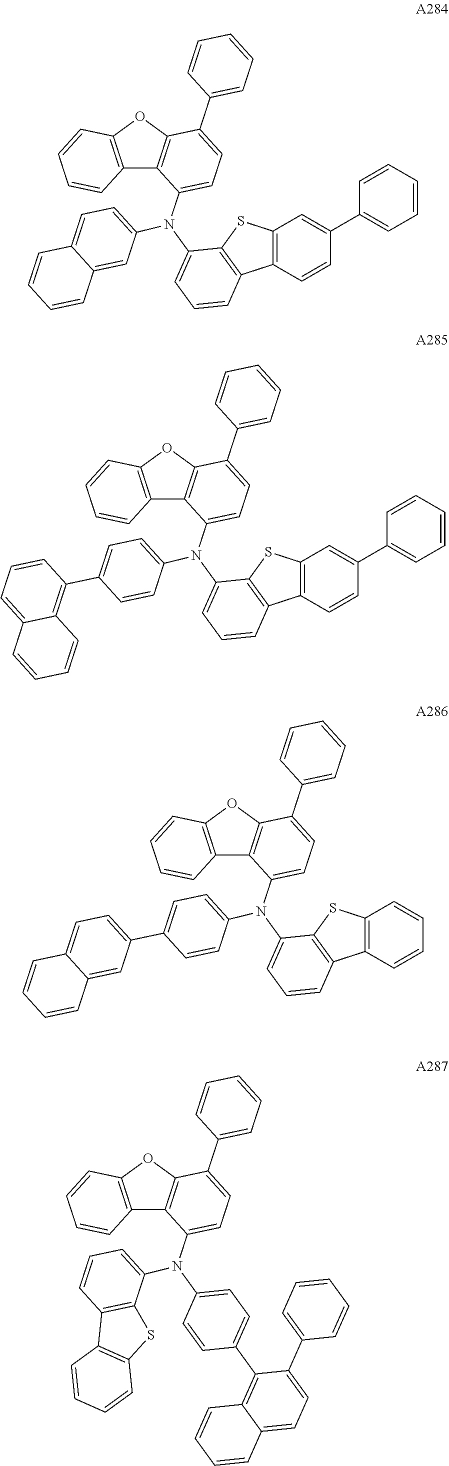 [SMILES: c1ccc(-c2ccc3c(c2)sc2c(N(c4ccc5ccccc5c4)c4ccc(-c5ccccc5)c5oc6ccccc6c45)cccc23)cc1 ; c1ccc(-c2ccc3c(c2)sc2c(N(c4ccc(-c5cccc6ccccc56)cc4)c4ccc(-c5ccccc5)c5oc6ccccc6c45)cccc23)cc1 ; c1ccc(-c2ccc(N(c3ccc(-c4ccc5ccccc5c4)cc3)c3cccc4c3sc3ccccc34)c3c2oc2ccccc23)cc1 ; c1ccc(-c2ccc3ccccc3c2-c2ccc(N(c3cccc4c3sc3ccccc34)c3ccc(-c4ccccc4)c4oc5ccccc5c34)cc2)cc1]

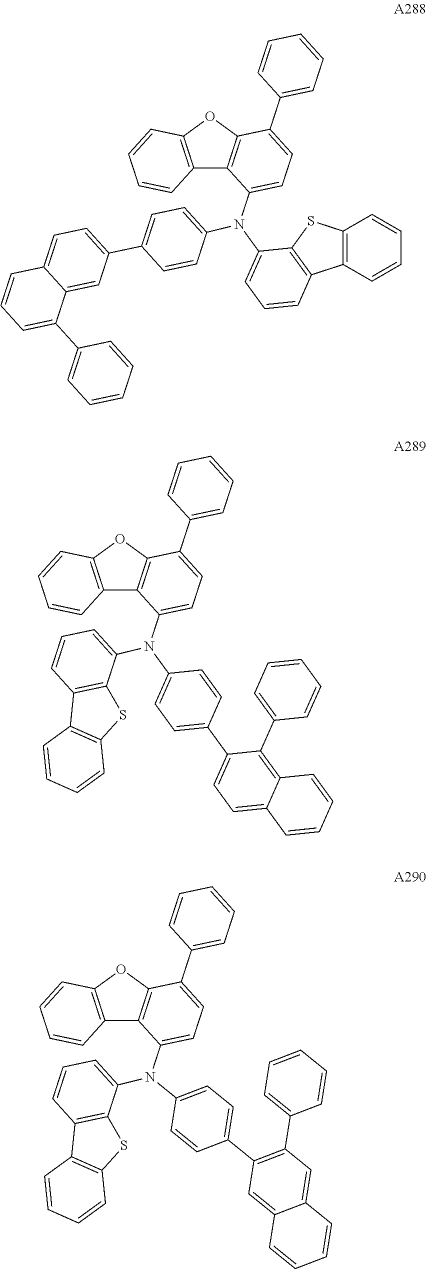 [SMILES: c1ccc(-c2cccc3ccc(-c4ccc(N(c5cccc6c5sc5ccccc56)c5ccc(-c6ccccc6)c6oc7ccccc7c56)cc4)cc23)cc1 ; c1ccc(-c2c(-c3ccc(N(c4cccc5c4sc4ccccc45)c4ccc(-c5ccccc5)c5oc6ccccc6c45)cc3)ccc3ccccc23)cc1 ; c1ccc(-c2cc3ccccc3cc2-c2ccc(N(c3cccc4c3sc3ccccc34)c3ccc(-c4ccccc4)c4oc5ccccc5c34)cc2)cc1]

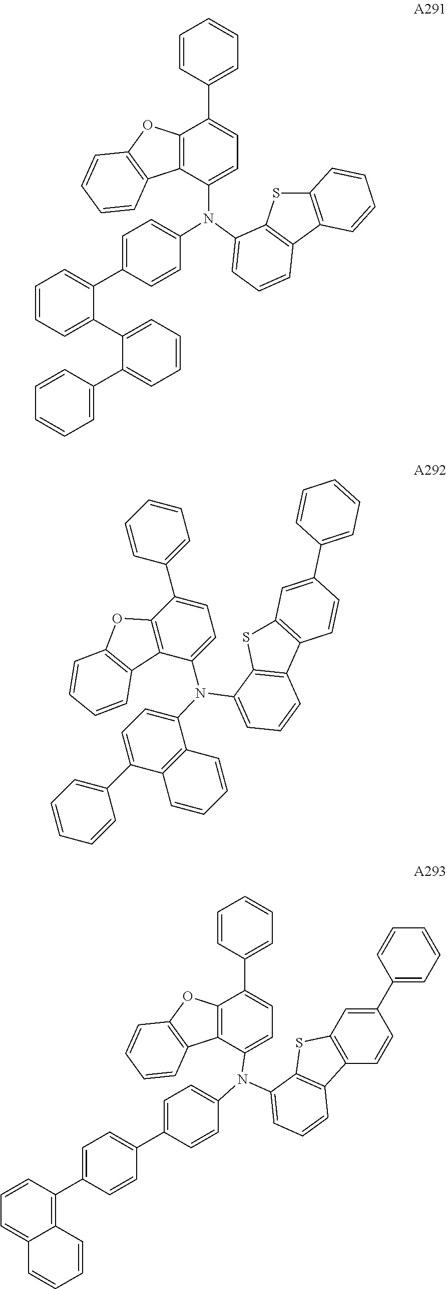 [SMILES: c1ccc(-c2ccccc2-c2ccccc2-c2ccc(N(c3cccc4c3sc3ccccc34)c3ccc(-c4ccccc4)c4oc5ccccc5c34)cc2)cc1 ; c1ccc(-c2ccc3c(c2)sc2c(N(c4ccc(-c5ccccc5)c5ccccc45)c4ccc(-c5ccccc5)c5oc6ccccc6c45)cccc23)cc1 ; c1ccc(-c2ccc3c(c2)sc2c(N(c4ccc(-c5ccc(-c6cccc7ccccc67)cc5)cc4)c4ccc(-c5ccccc5)c5oc6ccccc6c45)cccc23)cc1]

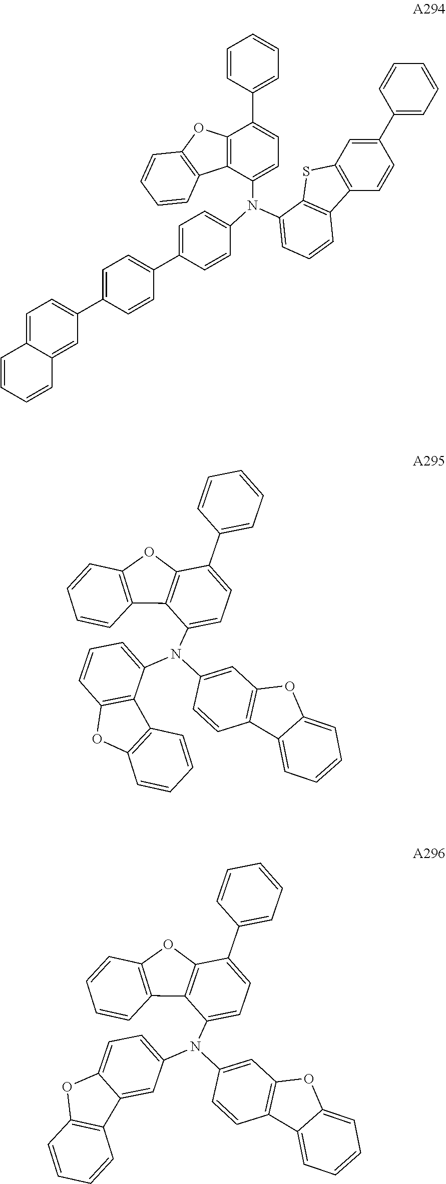 [SMILES: c1ccc(-c2ccc3c(c2)sc2c(N(c4ccc(-c5ccc(-c6ccc7ccccc7c6)cc5)cc4)c4ccc(-c5ccccc5)c5oc6ccccc6c45)cccc23)cc1 ; c1ccc(-c2ccc(N(c3ccc4c(c3)oc3ccccc34)c3cccc4oc5ccccc5c34)c3c2oc2ccccc23)cc1 ; c1ccc(-c2ccc(N(c3ccc4c(c3)oc3ccccc34)c3ccc4oc5ccccc5c4c3)c3c2oc2ccccc23)cc1]

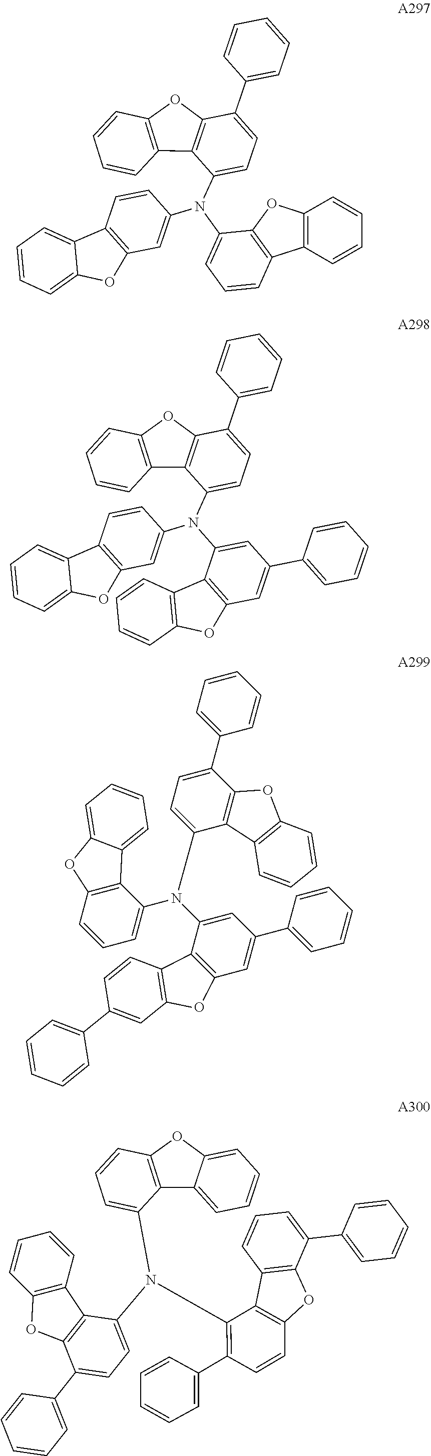

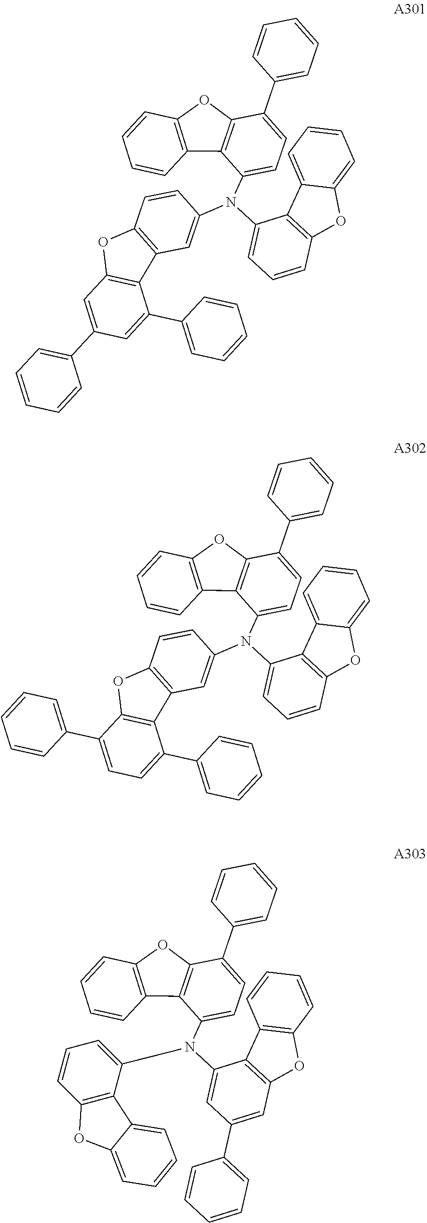 [SMILES: c1ccc(-c2cc(-c3ccccc3)c3c(c2)oc2ccc(N(c4cccc5oc6ccccc6c45)c4ccc(-c5ccccc5)c5oc6ccccc6c45)cc23)cc1 ; c1ccc(-c2ccc(-c3ccccc3)c3c2oc2ccc(N(c4cccc5oc6ccccc6c45)c4ccc(-c5ccccc5)c5oc6ccccc6c45)cc23)cc1 ; c1ccc(-c2cc(N(c3cccc4oc5ccccc5c34)c3ccc(-c4ccccc4)c4oc5ccccc5c34)c3c(c2)oc2ccccc23)cc1]

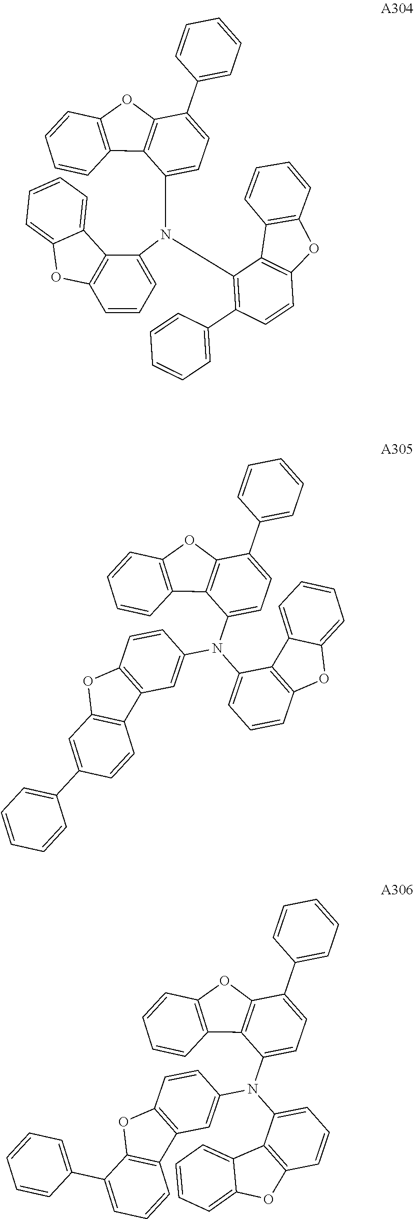 [SMILES: c1ccc(-c2ccc3oc4ccccc4c3c2N(c2cccc3oc4ccccc4c23)c2ccc(-c3ccccc3)c3oc4ccccc4c23)cc1 ; c1ccc(-c2ccc3c(c2)oc2ccc(N(c4cccc5oc6ccccc6c45)c4ccc(-c5ccccc5)c5oc6ccccc6c45)cc23)cc1 ; c1ccc(-c2cccc3c2oc2ccc(N(c4cccc5oc6ccccc6c45)c4ccc(-c5ccccc5)c5oc6ccccc6c45)cc23)cc1]

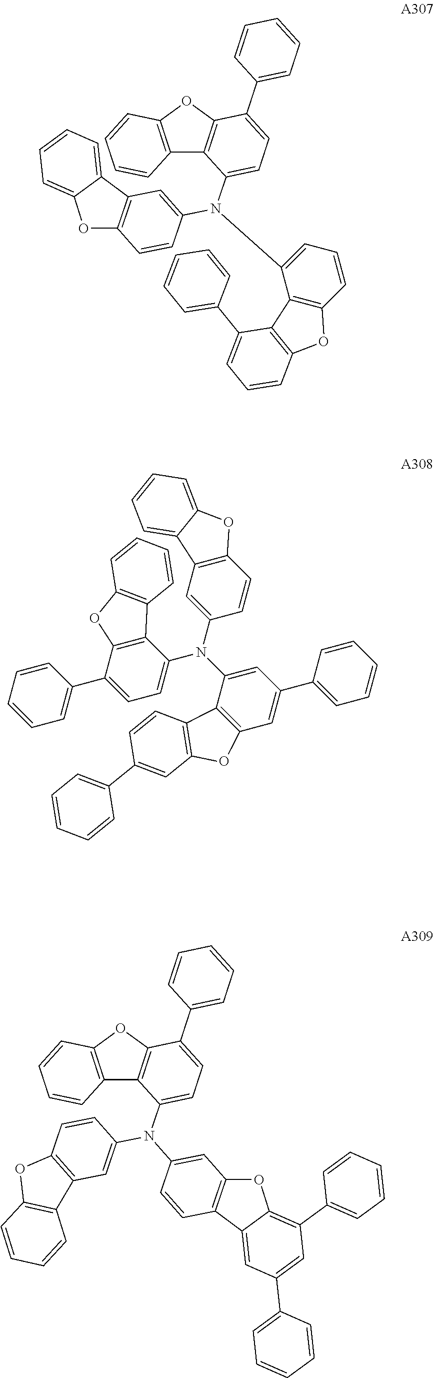 [SMILES: c1ccc(-c2ccc(N(c3ccc4oc5ccccc5c4c3)c3cccc4oc5cccc(-c6ccccc6)c5c34)c3c2oc2ccccc23)cc1 ; c1ccc(-c2ccc3c(c2)oc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3oc4cc(N(c5ccc6oc7ccccc7c6c5)c5ccc(-c6ccccc6)c6oc7ccccc7c56)ccc4c3c2)cc1]

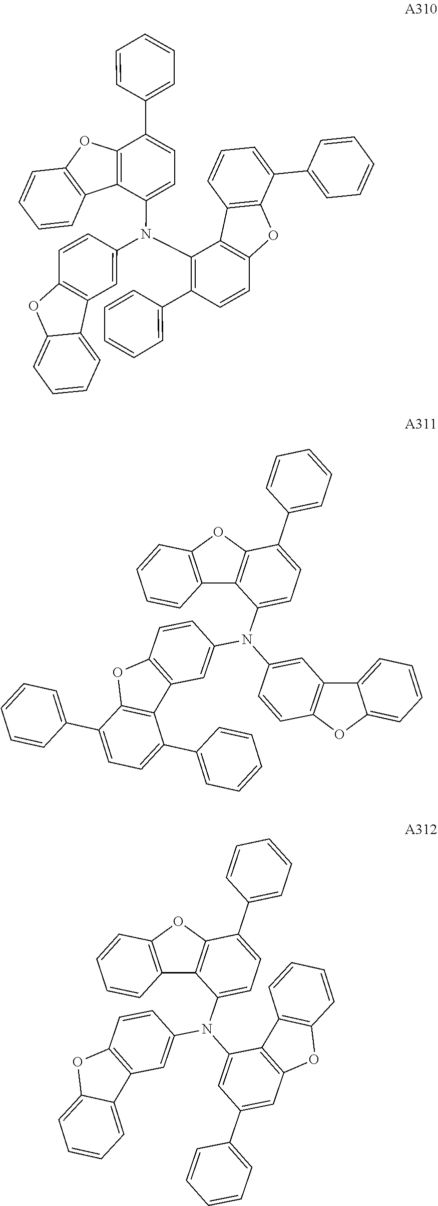 [SMILES: c1ccc(-c2ccc3oc4c(-c5ccccc5)cccc4c3c2N(c2ccc3oc4ccccc4c3c2)c2ccc(-c3ccccc3)c3oc4ccccc4c23)cc1 ; c1ccc(-c2ccc(-c3ccccc3)c3c2oc2ccc(N(c4ccc5oc6ccccc6c5c4)c4ccc(-c5ccccc5)c5oc6ccccc6c45)cc23)cc1 ; c1ccc(-c2cc(N(c3ccc4oc5ccccc5c4c3)c3ccc(-c4ccccc4)c4oc5ccccc5c34)c3c(c2)oc2ccccc23)cc1]

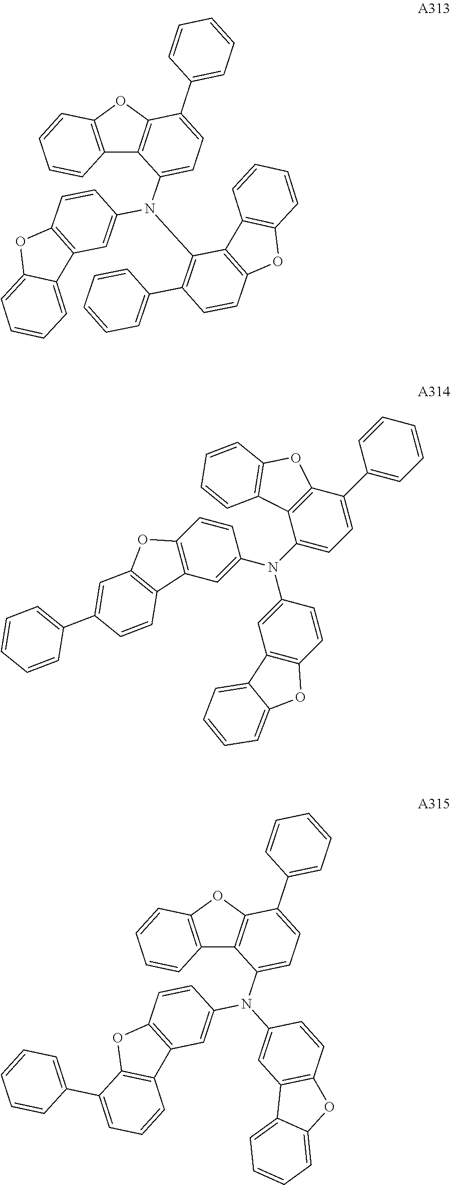 [SMILES: c1ccc(-c2ccc3oc4ccccc4c3c2N(c2ccc3oc4ccccc4c3c2)c2ccc(-c3ccccc3)c3oc4ccccc4c23)cc1 ; c1ccc(-c2ccc3c(c2)oc2ccc(N(c4ccc5oc6ccccc6c5c4)c4ccc(-c5ccccc5)c5oc6ccccc6c45)cc23)cc1 ; c1ccc(-c2cccc3c2oc2ccc(N(c4ccc5oc6ccccc6c5c4)c4ccc(-c5ccccc5)c5oc6ccccc6c45)cc23)cc1]

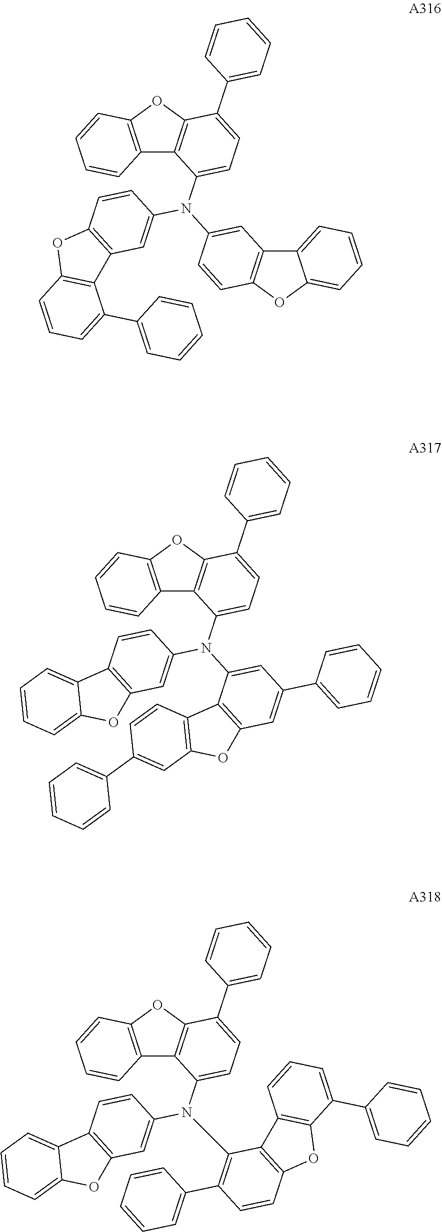 [SMILES: c1ccc(-c2ccc(N(c3ccc4oc5ccccc5c4c3)c3ccc4oc5cccc(-c6ccccc6)c5c4c3)c3c2oc2ccccc23)cc1 ; c1ccc(-c2ccc3c(c2)oc2cc(-c4ccccc4)cc(N(c4ccc5c(c4)oc4ccccc45)c4ccc(-c5ccccc5)c5oc6ccccc6c45)c23)cc1 ; c1ccc(-c2ccc3oc4c(-c5ccccc5)cccc4c3c2N(c2ccc3c(c2)oc2ccccc23)c2ccc(-c3ccccc3)c3oc4ccccc4c23)cc1]

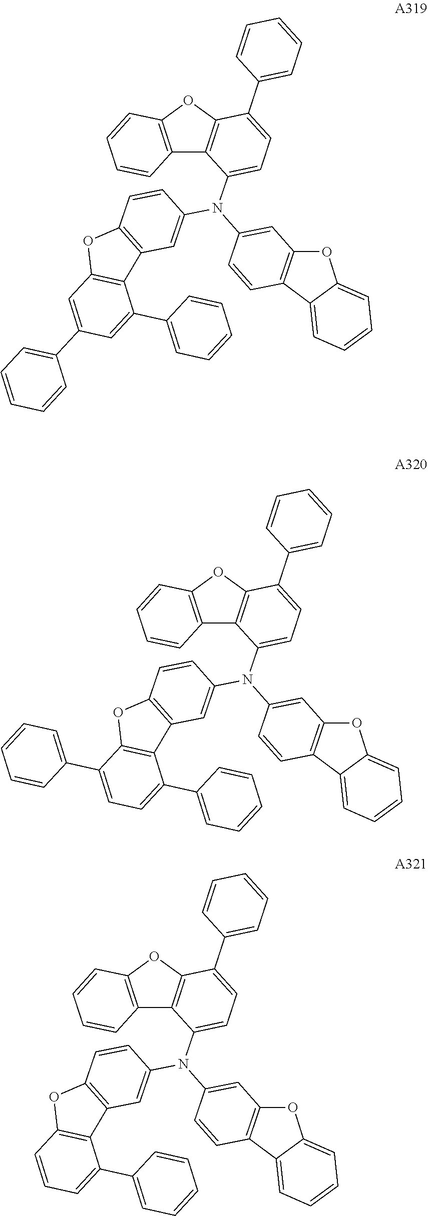

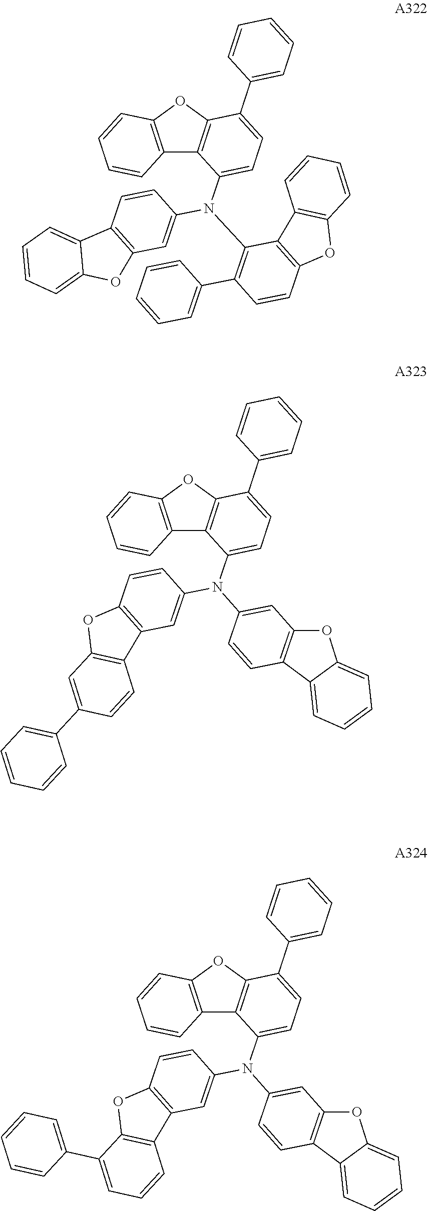 [SMILES: c1ccc(-c2ccc3oc4ccccc4c3c2N(c2ccc3c(c2)oc2ccccc23)c2ccc(-c3ccccc3)c3oc4ccccc4c23)cc1 ; c1ccc(-c2ccc3c(c2)oc2ccc(N(c4ccc5c(c4)oc4ccccc45)c4ccc(-c5ccccc5)c5oc6ccccc6c45)cc23)cc1 ; c1ccc(-c2cccc3c2oc2ccc(N(c4ccc5c(c4)oc4ccccc45)c4ccc(-c5ccccc5)c5oc6ccccc6c45)cc23)cc1]

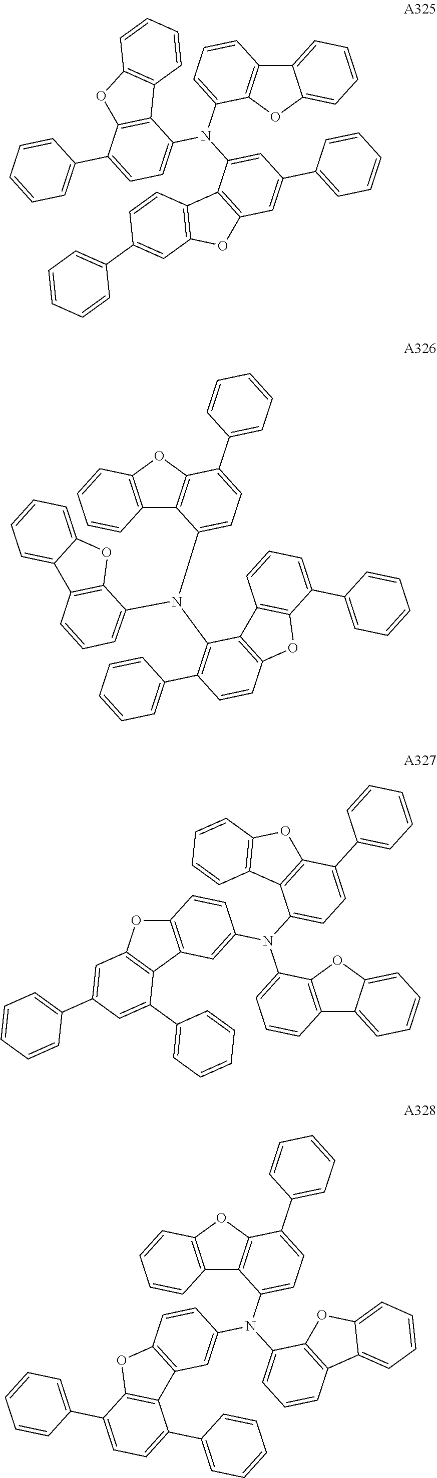 [SMILES: c1ccc(-c2ccc3c(c2)oc2cc(-c4ccccc4)cc(N(c4cccc5c4oc4ccccc45)c4ccc(-c5ccccc5)c5oc6ccccc6c45)c23)cc1 ; c1ccc(-c2ccc3oc4c(-c5ccccc5)cccc4c3c2N(c2cccc3c2oc2ccccc23)c2ccc(-c3ccccc3)c3oc4ccccc4c23)cc1 ; c1ccc(-c2cc(-c3ccccc3)c3c(c2)oc2ccc(N(c4cccc5c4oc4ccccc45)c4ccc(-c5ccccc5)c5oc6ccccc6c45)cc23)cc1 ; c1ccc(-c2ccc(-c3ccccc3)c3c2oc2ccc(N(c4cccc5c4oc4ccccc45)c4ccc(-c5ccccc5)c5oc6ccccc6c45)cc23)cc1]

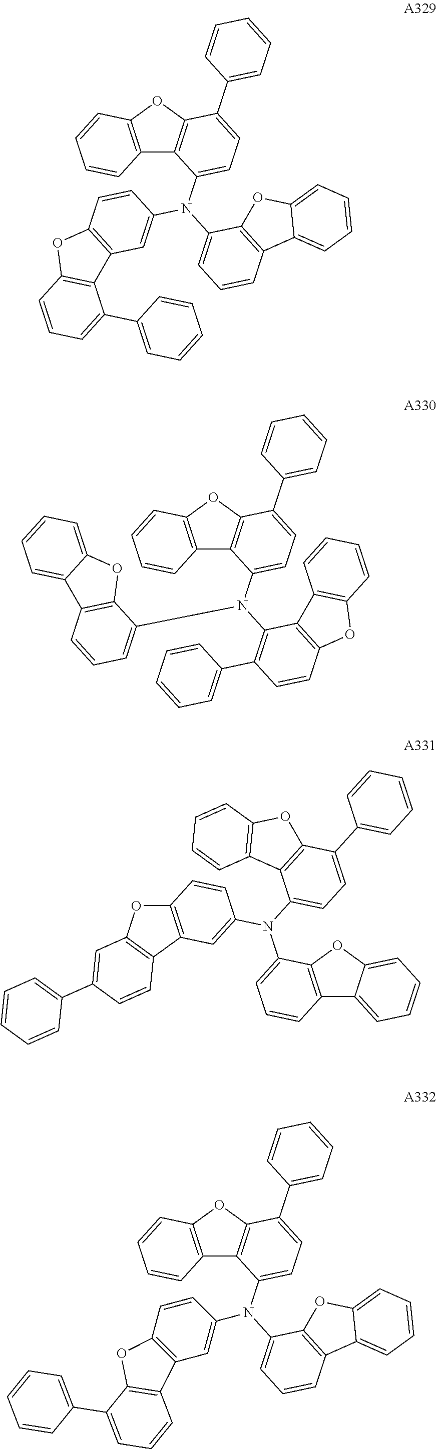 [SMILES: c1ccc(-c2ccc(N(c3ccc4oc5cccc(-c6ccccc6)c5c4c3)c3cccc4c3oc3ccccc34)c3c2oc2ccccc23)cc1 ; c1ccc(-c2ccc3oc4ccccc4c3c2N(c2cccc3c2oc2ccccc23)c2ccc(-c3ccccc3)c3oc4ccccc4c23)cc1 ; c1ccc(-c2ccc3c(c2)oc2ccc(N(c4cccc5c4oc4ccccc45)c4ccc(-c5ccccc5)c5oc6ccccc6c45)cc23)cc1 ; c1ccc(-c2cccc3c2oc2ccc(N(c4cccc5c4oc4ccccc45)c4ccc(-c5ccccc5)c5oc6ccccc6c45)cc23)cc1]

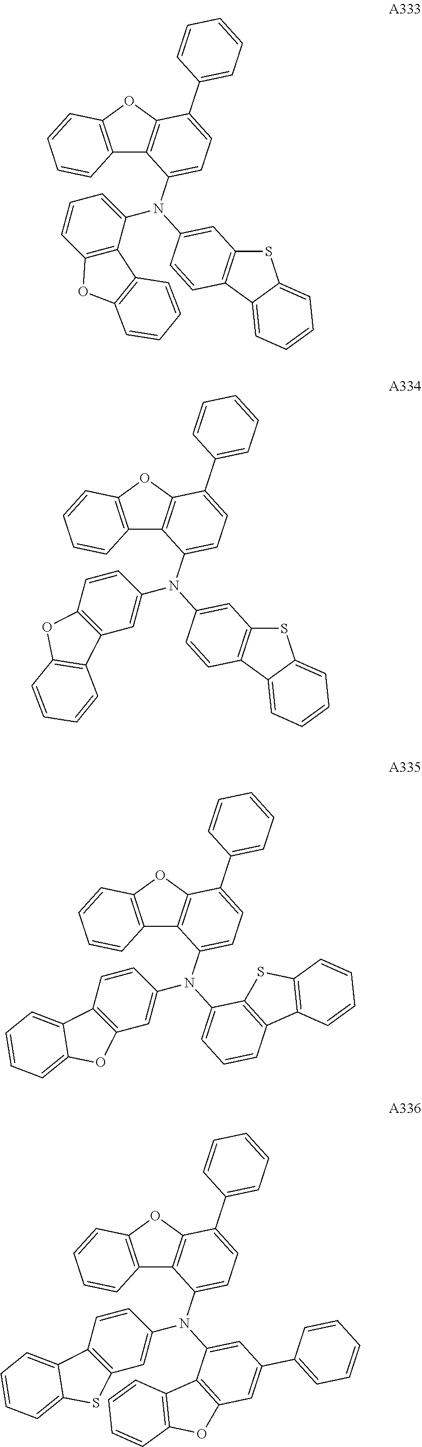 [SMILES: c1ccc(-c2ccc(N(c3ccc4c(c3)sc3ccccc34)c3cccc4oc5ccccc5c34)c3c2oc2ccccc23)cc1 ; c1ccc(-c2ccc(N(c3ccc4c(c3)sc3ccccc34)c3ccc4oc5ccccc5c4c3)c3c2oc2ccccc23)cc1 ; c1ccc(-c2ccc(N(c3ccc4c(c3)oc3ccccc34)c3cccc4c3sc3ccccc34)c3c2oc2ccccc23)cc1 ; c1ccc(-c2cc(N(c3ccc4c(c3)sc3ccccc34)c3ccc(-c4ccccc4)c4oc5ccccc5c34)c3c(c2)oc2ccccc23)cc1]

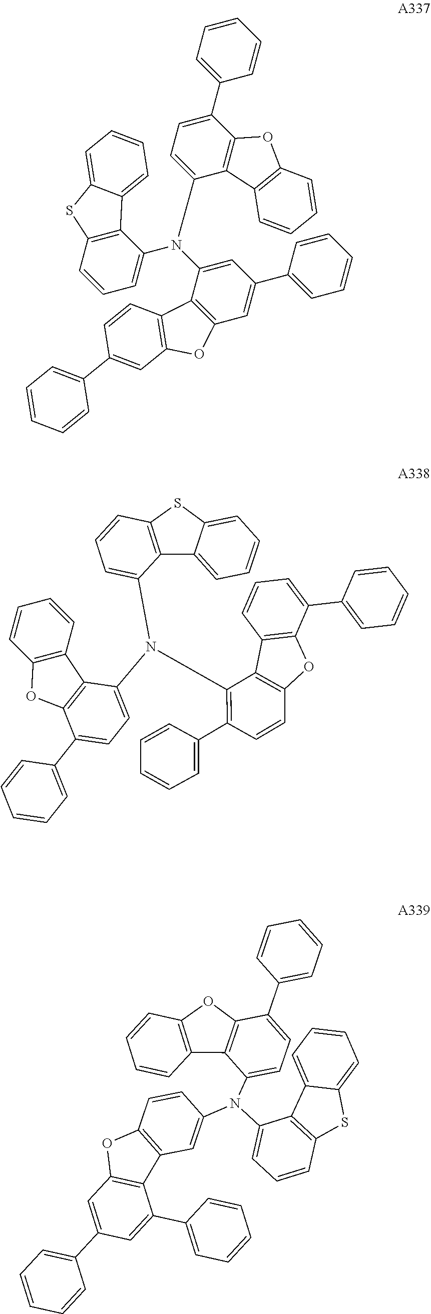 [SMILES: c1ccc(-c2ccc3c(c2)oc2cc(-c4ccccc4)cc(N(c4cccc5sc6ccccc6c45)c4ccc(-c5ccccc5)c5oc6ccccc6c45)c23)cc1 ; c1ccc(-c2ccc3oc4c(-c5ccccc5)cccc4c3c2N(c2cccc3sc4ccccc4c23)c2ccc(-c3ccccc3)c3oc4ccccc4c23)cc1 ; c1ccc(-c2cc(-c3ccccc3)c3c(c2)oc2ccc(N(c4cccc5sc6ccccc6c45)c4ccc(-c5ccccc5)c5oc6ccccc6c45)cc23)cc1]

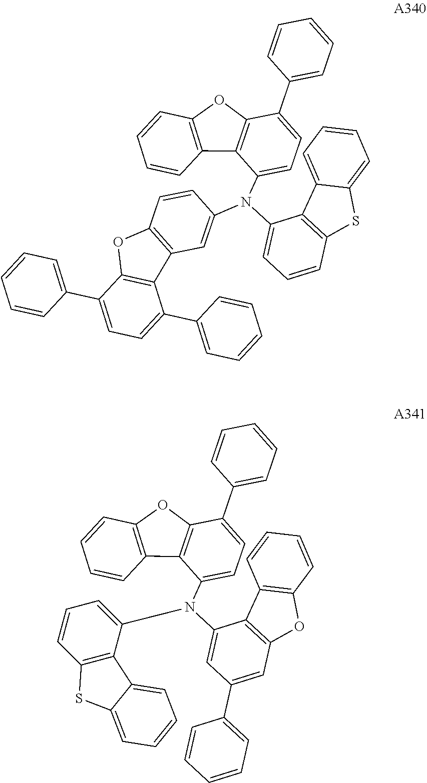 [SMILES: c1ccc(-c2ccc(-c3ccccc3)c3c2oc2ccc(N(c4cccc5sc6ccccc6c45)c4ccc(-c5ccccc5)c5oc6ccccc6c45)cc23)cc1 ; c1ccc(-c2cc(N(c3cccc4sc5ccccc5c34)c3ccc(-c4ccccc4)c4oc5ccccc5c34)c3c(c2)oc2ccccc23)cc1]

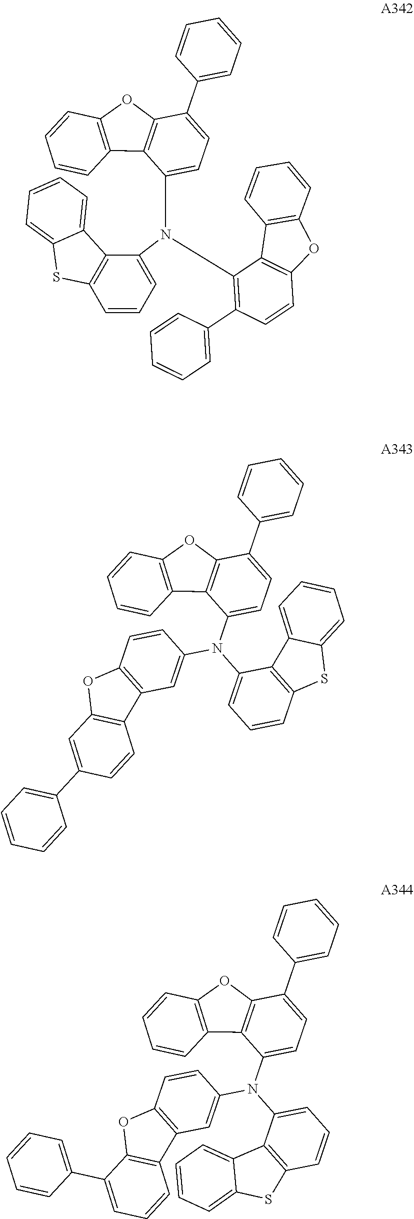 [SMILES: c1ccc(-c2ccc3oc4ccccc4c3c2N(c2cccc3sc4ccccc4c23)c2ccc(-c3ccccc3)c3oc4ccccc4c23)cc1 ; c1ccc(-c2ccc3c(c2)oc2ccc(N(c4cccc5sc6ccccc6c45)c4ccc(-c5ccccc5)c5oc6ccccc6c45)cc23)cc1 ; c1ccc(-c2cccc3c2oc2ccc(N(c4cccc5sc6ccccc6c45)c4ccc(-c5ccccc5)c5oc6ccccc6c45)cc23)cc1]

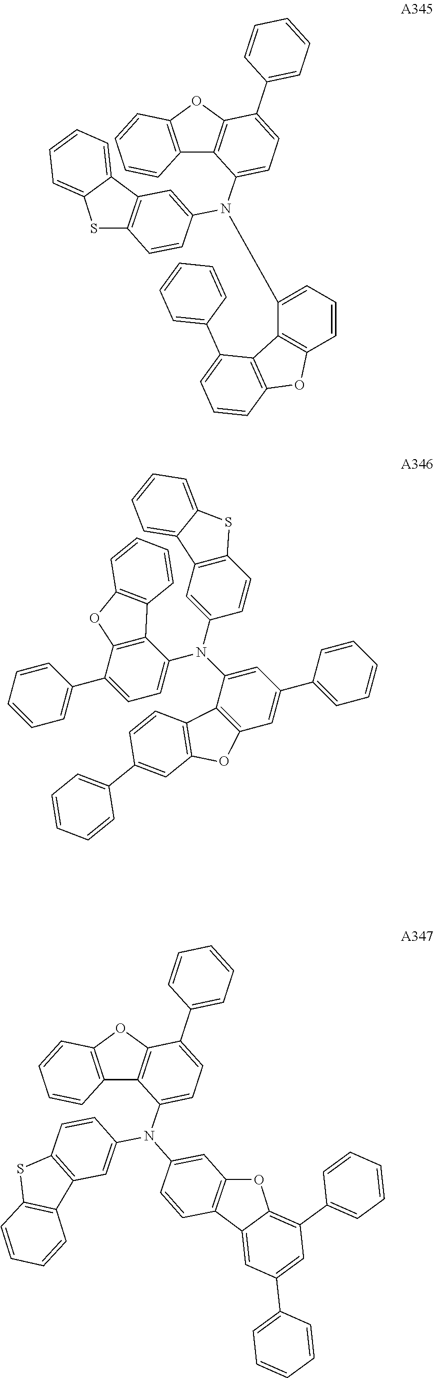 [SMILES: c1ccc(-c2ccc(N(c3ccc4sc5ccccc5c4c3)c3cccc4oc5cccc(-c6ccccc6)c5c34)c3c2oc2ccccc23)cc1 ; c1ccc(-c2ccc3c(c2)oc2cc(-c4ccccc4)cc(N(c4ccc5sc6ccccc6c5c4)c4ccc(-c5ccccc5)c5oc6ccccc6c45)c23)cc1 ; c1ccc(-c2cc(-c3ccccc3)c3oc4cc(N(c5ccc6sc7ccccc7c6c5)c5ccc(-c6ccccc6)c6oc7ccccc7c56)ccc4c3c2)cc1]

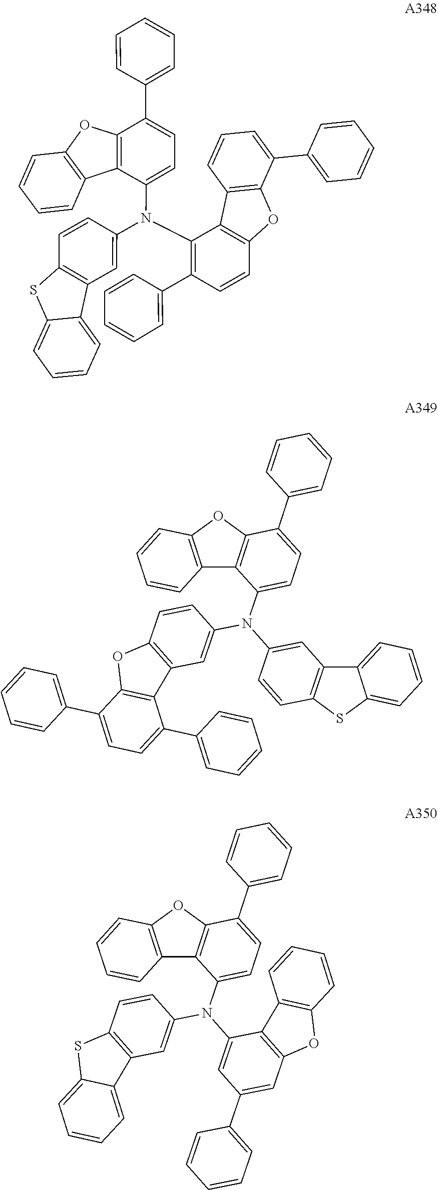 [SMILES: c1ccc(-c2ccc3oc4c(-c5ccccc5)cccc4c3c2N(c2ccc3sc4ccccc4c3c2)c2ccc(-c3ccccc3)c3oc4ccccc4c23)cc1 ; c1ccc(-c2ccc(-c3ccccc3)c3c2oc2ccc(N(c4ccc5sc6ccccc6c5c4)c4ccc(-c5ccccc5)c5oc6ccccc6c45)cc23)cc1 ; c1ccc(-c2cc(N(c3ccc4sc5ccccc5c4c3)c3ccc(-c4ccccc4)c4oc5ccccc5c34)c3c(c2)oc2ccccc23)cc1]

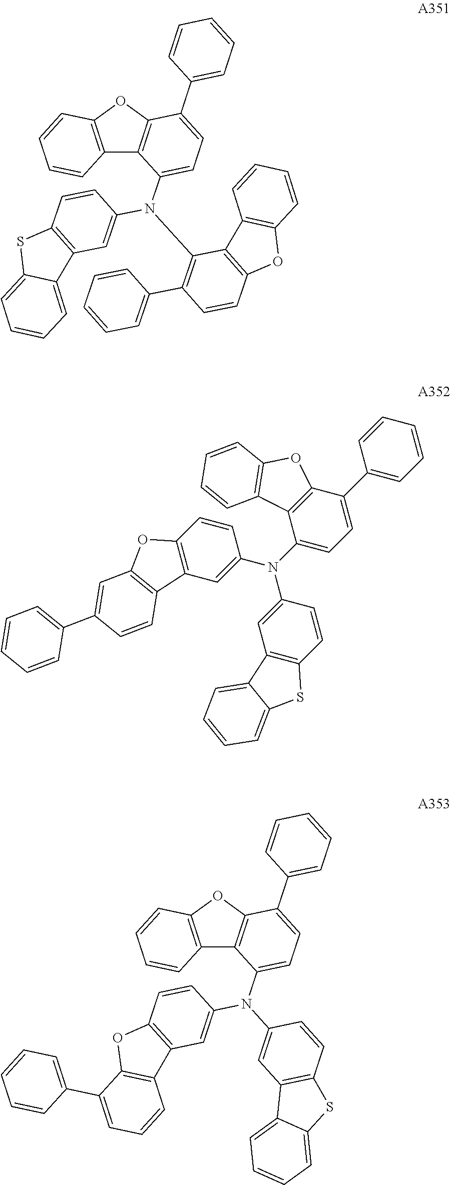 [SMILES: c1ccc(-c2ccc3oc4ccccc4c3c2N(c2ccc3sc4ccccc4c3c2)c2ccc(-c3ccccc3)c3oc4ccccc4c23)cc1 ; c1ccc(-c2ccc3c(c2)oc2ccc(N(c4ccc5sc6ccccc6c5c4)c4ccc(-c5ccccc5)c5oc6ccccc6c45)cc23)cc1 ; c1ccc(-c2cccc3c2oc2ccc(N(c4ccc5sc6ccccc6c5c4)c4ccc(-c5ccccc5)c5oc6ccccc6c45)cc23)cc1]

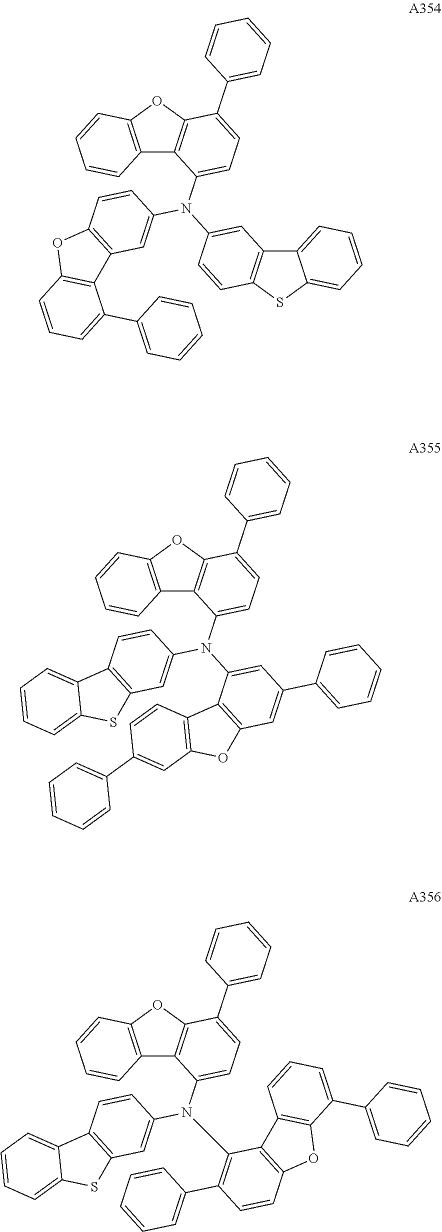 [SMILES: c1ccc(-c2ccc(N(c3ccc4sc5ccccc5c4c3)c3ccc4oc5cccc(-c6ccccc6)c5c4c3)c3c2oc2ccccc23)cc1 ; c1ccc(-c2ccc3c(c2)oc2cc(-c4ccccc4)cc(N(c4ccc5c(c4)sc4ccccc45)c4ccc(-c5ccccc5)c5oc6ccccc6c45)c23)cc1 ; c1ccc(-c2ccc3oc4c(-c5ccccc5)cccc4c3c2N(c2ccc3c(c2)sc2ccccc23)c2ccc(-c3ccccc3)c3oc4ccccc4c23)cc1]

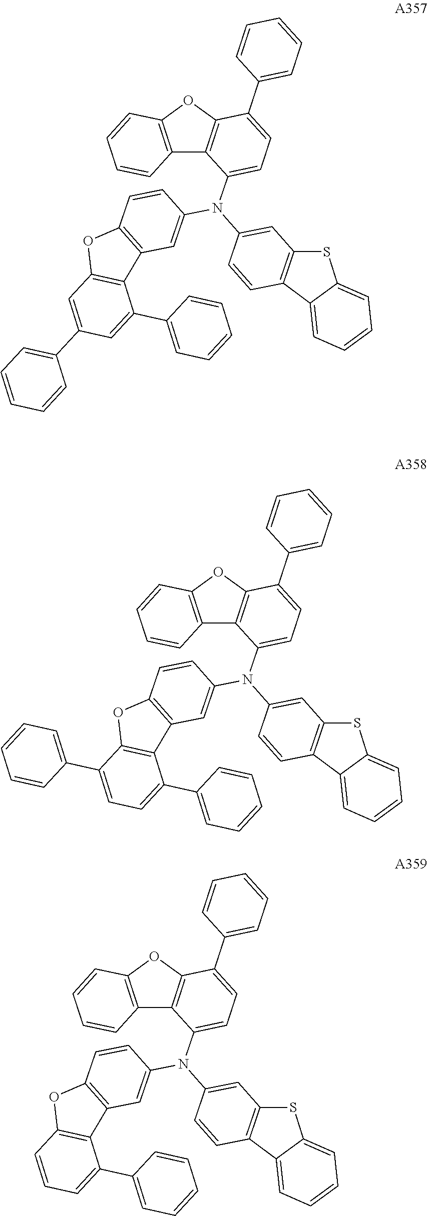

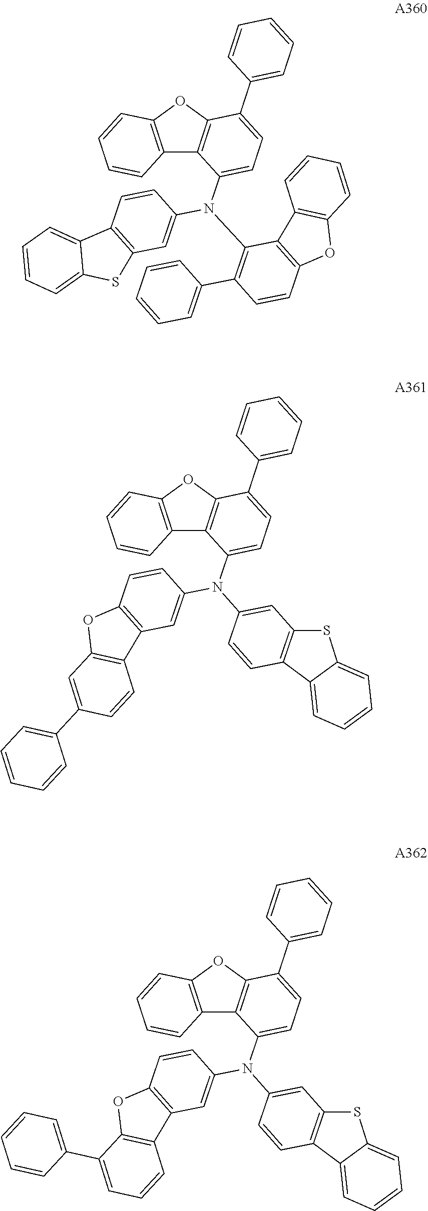 [SMILES: c1ccc(-c2ccc3oc4ccccc4c3c2N(c2ccc3c(c2)sc2ccccc23)c2ccc(-c3ccccc3)c3oc4ccccc4c23)cc1 ; c1ccc(-c2ccc3c(c2)oc2ccc(N(c4ccc5c(c4)sc4ccccc45)c4ccc(-c5ccccc5)c5oc6ccccc6c45)cc23)cc1 ; c1ccc(-c2cccc3c2oc2ccc(N(c4ccc5c(c4)sc4ccccc45)c4ccc(-c5ccccc5)c5oc6ccccc6c45)cc23)cc1]

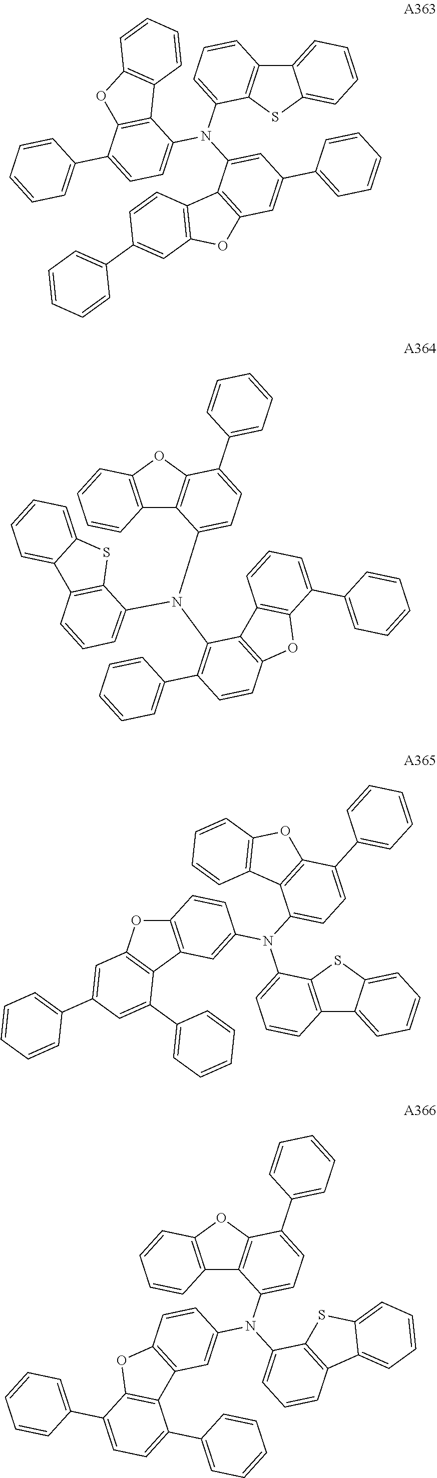 [SMILES: c1ccc(-c2ccc3c(c2)oc2cc(-c4ccccc4)cc(N(c4cccc5c4sc4ccccc45)c4ccc(-c5ccccc5)c5oc6ccccc6c45)c23)cc1 ; c1ccc(-c2ccc3oc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)oc2ccc(N(c4cccc5c4sc4ccccc45)c4ccc(-c5ccccc5)c5oc6ccccc6c45)cc23)cc1 ; c1ccc(-c2ccc(-c3ccccc3)c3c2oc2ccc(N(c4cccc5c4sc4ccccc45)c4ccc(-c5ccccc5)c5oc6ccccc6c45)cc23)cc1]

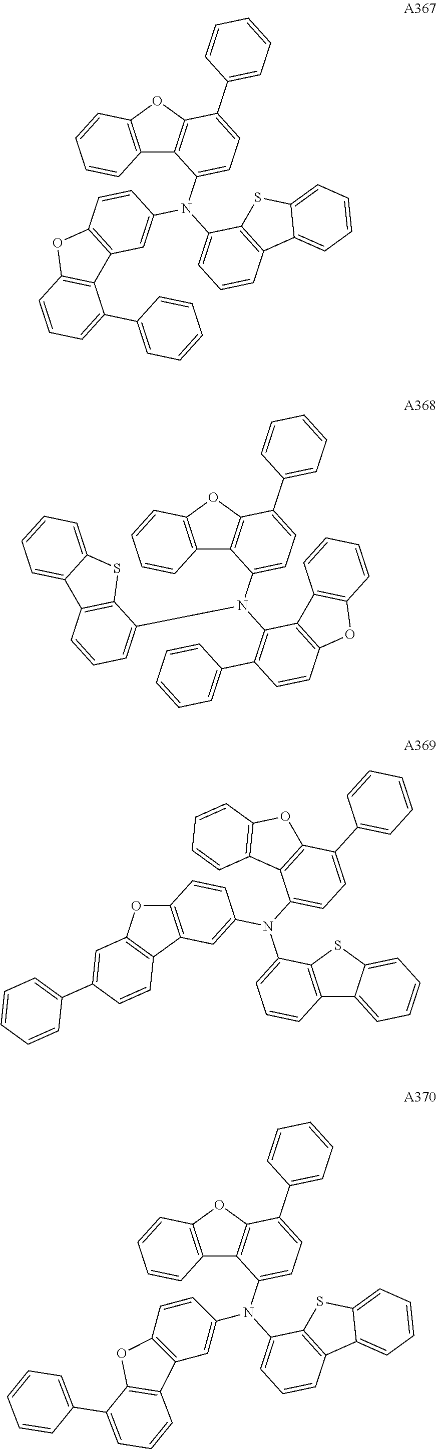 [SMILES: c1ccc(-c2ccc(N(c3ccc4oc5cccc(-c6ccccc6)c5c4c3)c3cccc4c3sc3ccccc34)c3c2oc2ccccc23)cc1 ; c1ccc(-c2ccc3oc4ccccc4c3c2N(c2cccc3c2sc2ccccc23)c2ccc(-c3ccccc3)c3oc4ccccc4c23)cc1 ; c1ccc(-c2ccc3c(c2)oc2ccc(N(c4cccc5c4sc4ccccc45)c4ccc(-c5ccccc5)c5oc6ccccc6c45)cc23)cc1 ; c1ccc(-c2cccc3c2oc2ccc(N(c4cccc5c4sc4ccccc45)c4ccc(-c5ccccc5)c5oc6ccccc6c45)cc23)cc1]

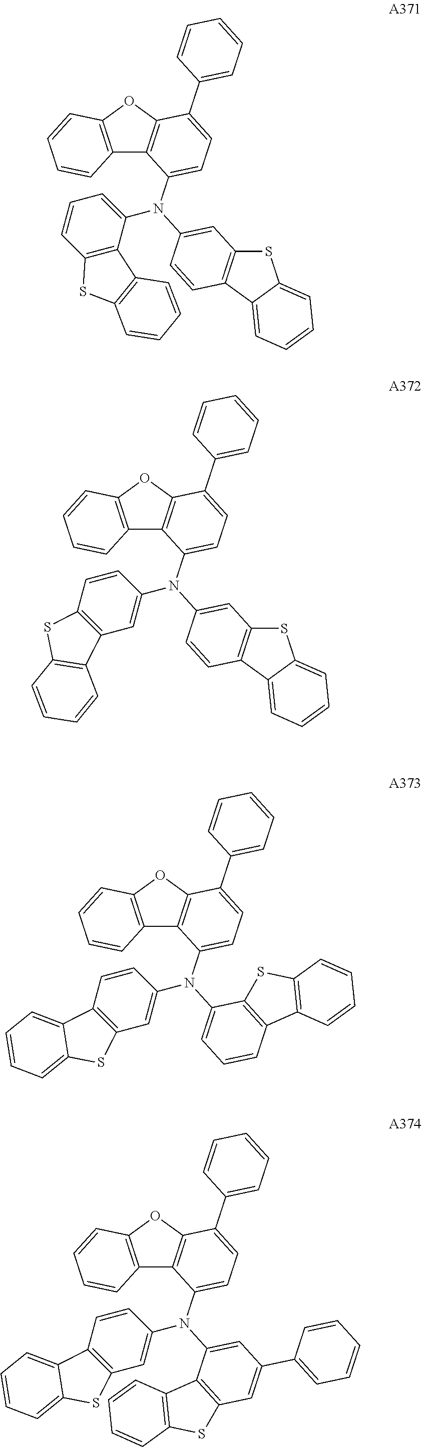 [SMILES: c1ccc(-c2ccc(N(c3ccc4c(c3)sc3ccccc34)c3cccc4sc5ccccc5c34)c3c2oc2ccccc23)cc1 ; c1ccc(-c2ccc(N(c3ccc4c(c3)sc3ccccc34)c3ccc4sc5ccccc5c4c3)c3c2oc2ccccc23)cc1 ; c1ccc(-c2ccc(N(c3ccc4c(c3)sc3ccccc34)c3cccc4c3sc3ccccc34)c3c2oc2ccccc23)cc1 ; c1ccc(-c2cc(N(c3ccc4c(c3)sc3ccccc34)c3ccc(-c4ccccc4)c4oc5ccccc5c34)c3c(c2)sc2ccccc23)cc1]

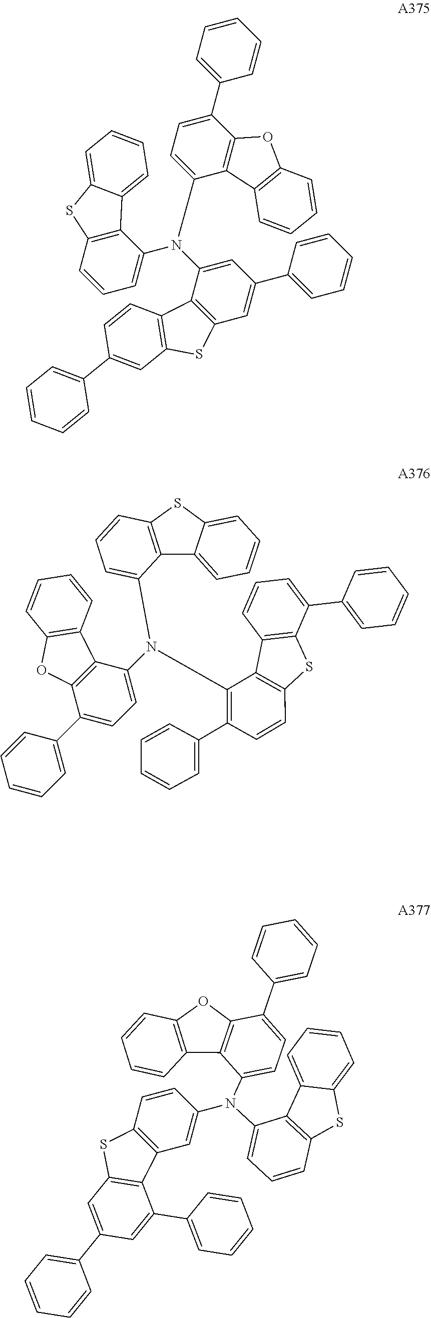 [SMILES: c1ccc(-c2ccc3c(c2)sc2cc(-c4ccccc4)cc(N(c4cccc5sc6ccccc6c45)c4ccc(-c5ccccc5)c5oc6ccccc6c45)c23)cc1 ; c1ccc(-c2ccc3sc4c(-c5ccccc5)cccc4c3c2N(c2cccc3sc4ccccc4c23)c2ccc(-c3ccccc3)c3oc4ccccc4c23)cc1 ; c1ccc(-c2cc(-c3ccccc3)c3c(c2)sc2ccc(N(c4cccc5sc6ccccc6c45)c4ccc(-c5ccccc5)c5oc6ccccc6c45)cc23)cc1]

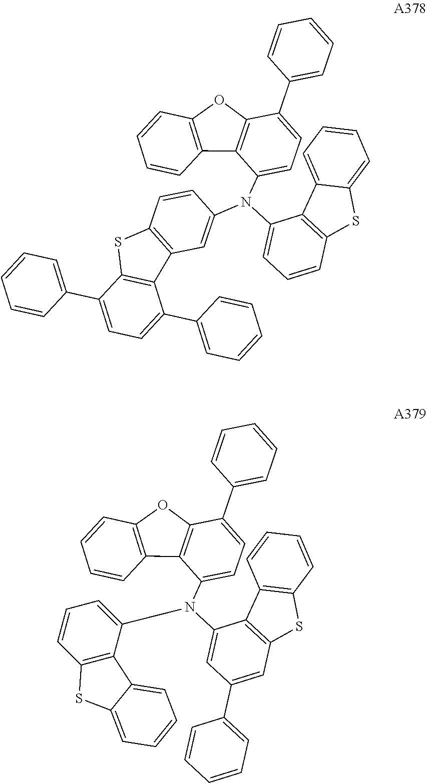 [SMILES: c1ccc(-c2ccc(N(c3ccc4sc5c(-c6ccccc6)ccc(-c6ccccc6)c5c4c3)c3cccc4sc5ccccc5c34)c3c2oc2ccccc23)cc1 ; c1ccc(-c2cc(N(c3cccc4sc5ccccc5c34)c3ccc(-c4ccccc4)c4oc5ccccc5c34)c3c(c2)sc2ccccc23)cc1]

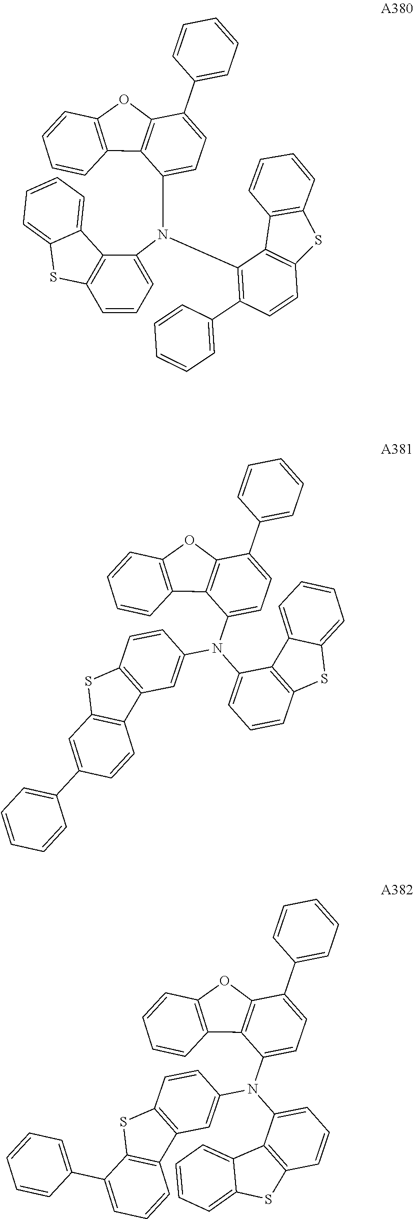 [SMILES: c1ccc(-c2ccc3sc4ccccc4c3c2N(c2cccc3sc4ccccc4c23)c2ccc(-c3ccccc3)c3oc4ccccc4c23)cc1 ; c1ccc(-c2ccc3c(c2)sc2ccc(N(c4cccc5sc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4sc5ccccc5c34)c3c2oc2ccccc23)cc1]

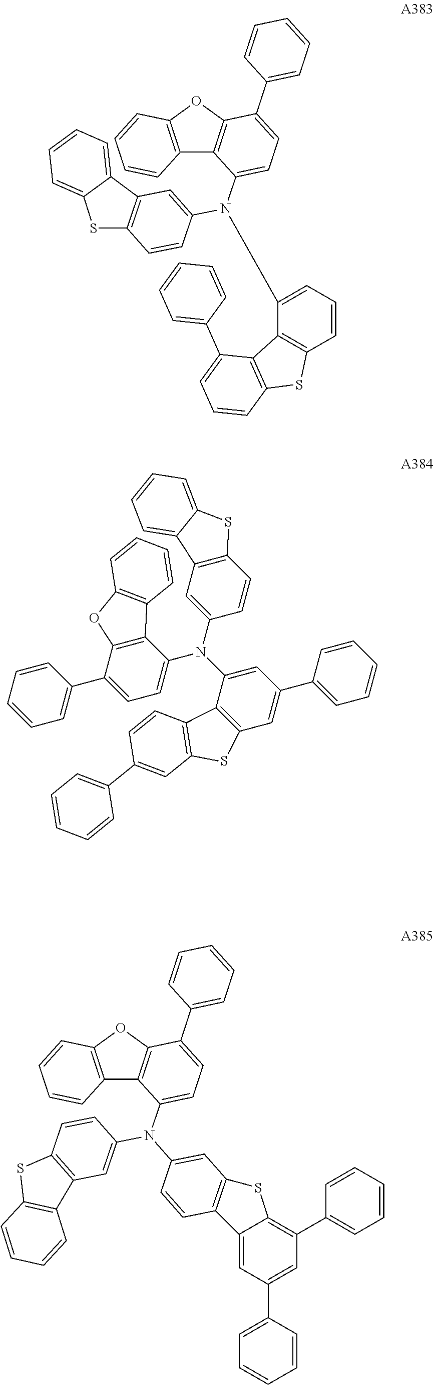 [SMILES: c1ccc(-c2ccc(N(c3ccc4sc5ccccc5c4c3)c3cccc4sc5cccc(-c6ccccc6)c5c34)c3c2oc2ccccc23)cc1 ; c1ccc(-c2ccc3c(c2)sc2cc(-c4ccccc4)cc(N(c4ccc5sc6ccccc6c5c4)c4ccc(-c5ccccc5)c5oc6ccccc6c45)c23)cc1 ; c1ccc(-c2cc(-c3ccccc3)c3sc4cc(N(c5ccc6sc7ccccc7c6c5)c5ccc(-c6ccccc6)c6oc7ccccc7c56)ccc4c3c2)cc1]

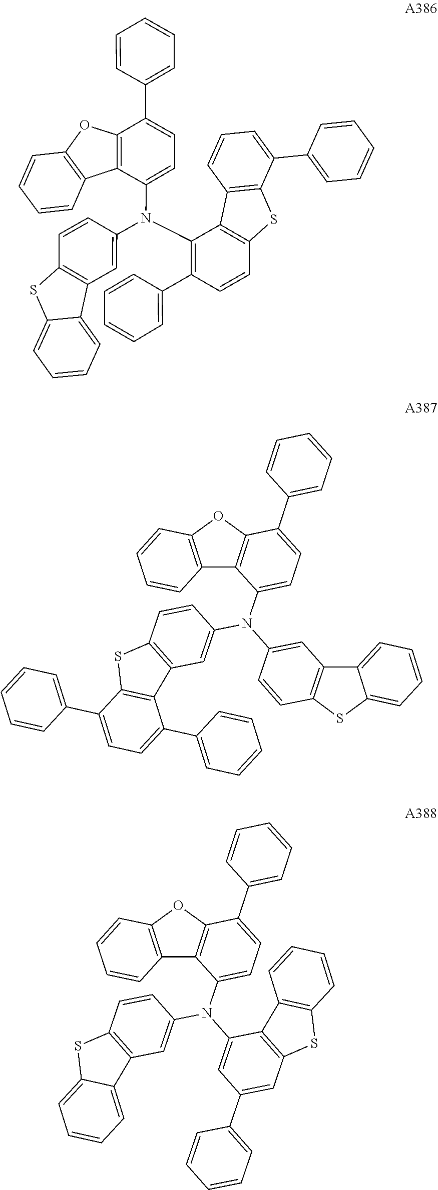 [SMILES: c1ccc(-c2ccc3sc4c(-c5ccccc5)cccc4c3c2N(c2ccc3sc4ccccc4c3c2)c2ccc(-c3ccccc3)c3oc4ccccc4c23)cc1 ; c1ccc(-c2ccc(N(c3ccc4sc5ccccc5c4c3)c3ccc4sc5c(-c6ccccc6)ccc(-c6ccccc6)c5c4c3)c3c2oc2ccccc23)cc1 ; c1ccc(-c2cc(N(c3ccc4sc5ccccc5c4c3)c3ccc(-c4ccccc4)c4oc5ccccc5c34)c3c(c2)sc2ccccc23)cc1]

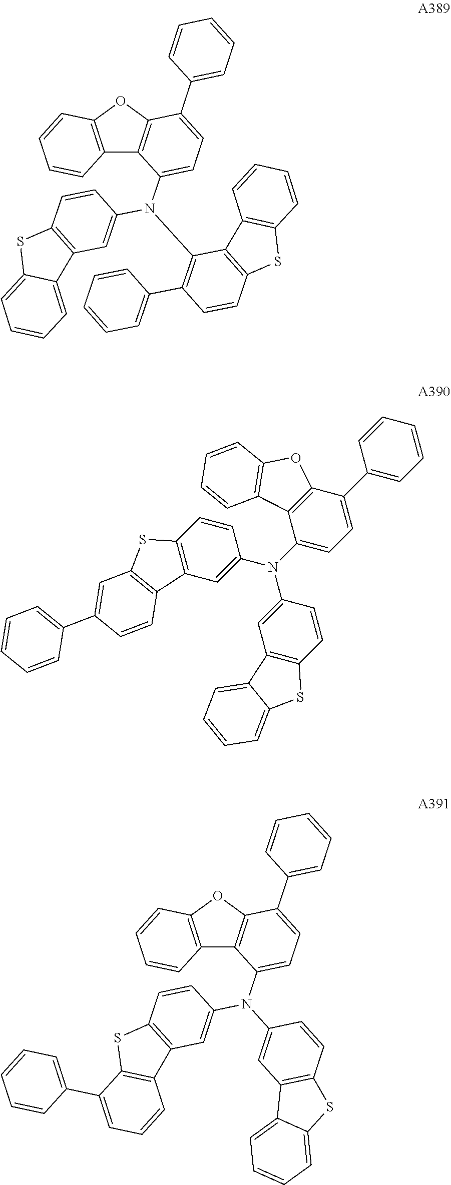 [SMILES: c1ccc(-c2ccc3sc4ccccc4c3c2N(c2ccc3sc4ccccc4c3c2)c2ccc(-c3ccccc3)c3oc4ccccc4c23)cc1 ; c1ccc(-c2ccc3c(c2)sc2ccc(N(c4ccc5sc6ccccc6c5c4)c4ccc(-c5ccccc5)c5oc6ccccc6c45)cc23)cc1 ; c1ccc(-c2ccc(N(c3ccc4sc5ccccc5c4c3)c3ccc4sc5c(-c6ccccc6)cccc5c4c3)c3c2oc2ccccc23)cc1]

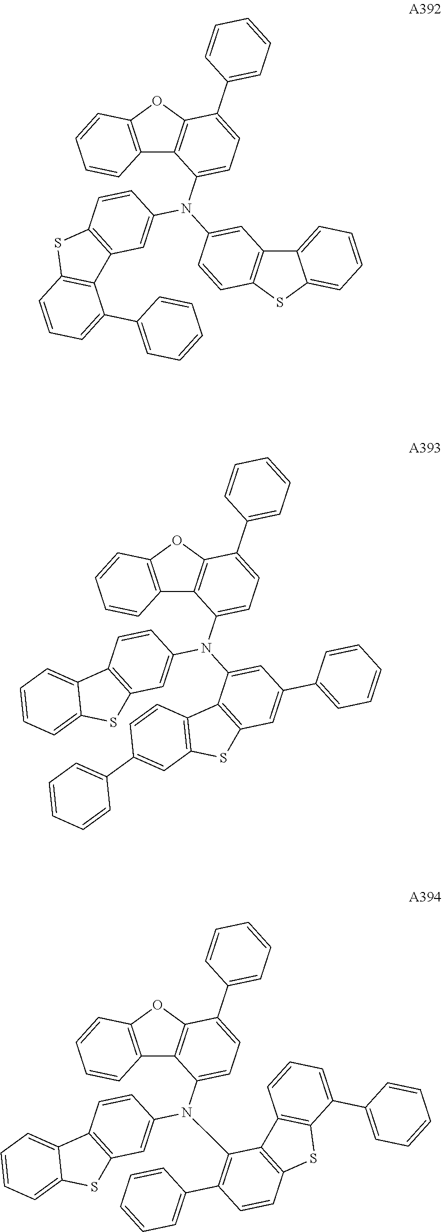 [SMILES: c1ccc(-c2ccc(N(c3ccc4sc5ccccc5c4c3)c3ccc4sc5cccc(-c6ccccc6)c5c4c3)c3c2oc2ccccc23)cc1 ; c1ccc(-c2ccc3c(c2)sc2cc(-c4ccccc4)cc(N(c4ccc5c(c4)sc4ccccc45)c4ccc(-c5ccccc5)c5oc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3oc4ccccc4c23)cc1]

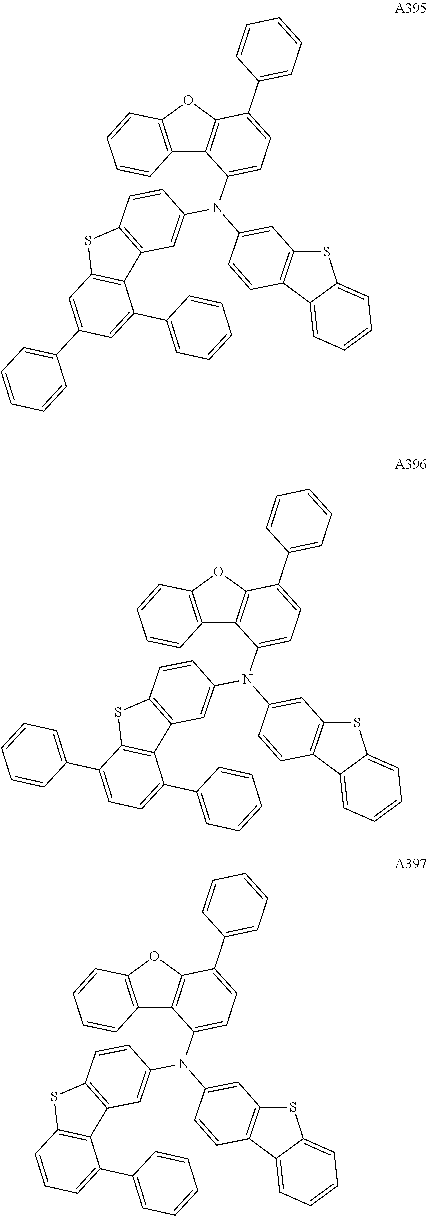

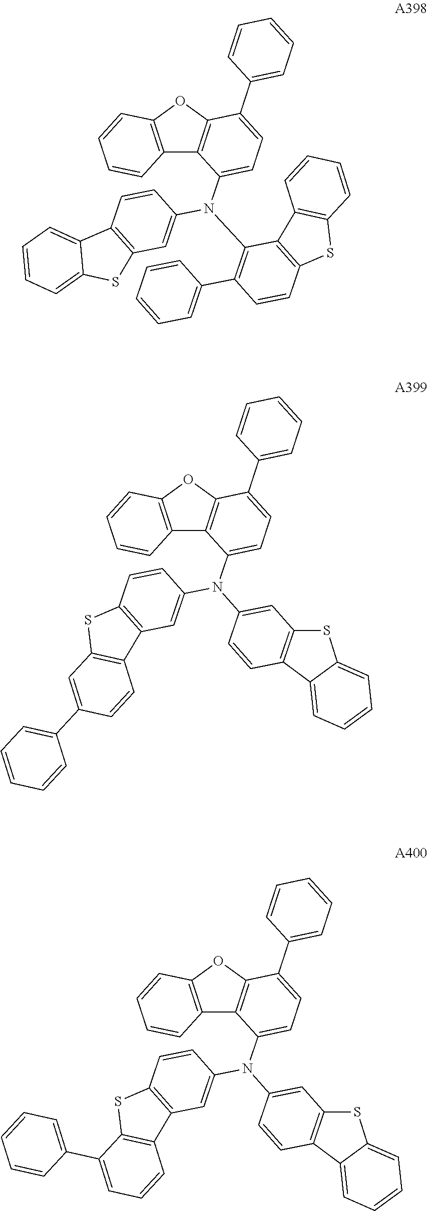 [SMILES: c1ccc(-c2ccc3sc4ccccc4c3c2N(c2ccc3c(c2)sc2ccccc23)c2ccc(-c3ccccc3)c3oc4ccccc4c23)cc1 ; c1ccc(-c2ccc3c(c2)sc2ccc(N(c4ccc5c(c4)sc4ccccc45)c4ccc(-c5ccccc5)c5oc6ccccc6c45)cc23)cc1 ; c1ccc(-c2ccc(N(c3ccc4c(c3)sc3ccccc34)c3ccc4sc5c(-c6ccccc6)cccc5c4c3)c3c2oc2ccccc23)cc1]

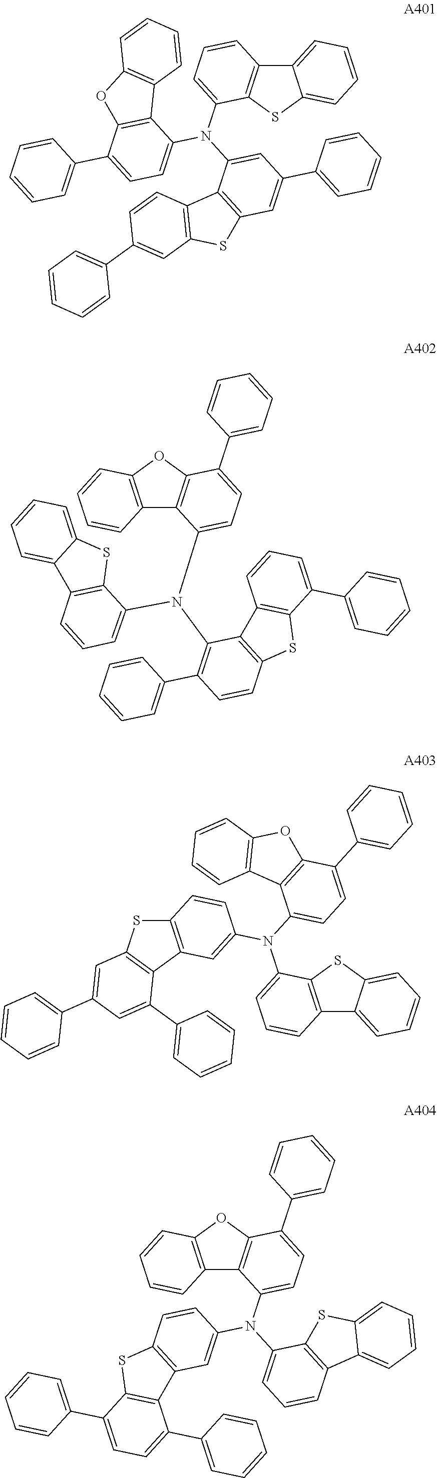 [SMILES: c1ccc(-c2ccc3c(c2)sc2cc(-c4ccccc4)cc(N(c4cccc5c4sc4ccccc45)c4ccc(-c5ccccc5)c5oc6ccccc6c45)c23)cc1 ; c1ccc(-c2ccc3sc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)sc2ccc(N(c4cccc5c4sc4ccccc45)c4ccc(-c5ccccc5)c5oc6ccccc6c45)cc23)cc1 ; c1ccc(-c2ccc(N(c3ccc4sc5c(-c6ccccc6)ccc(-c6ccccc6)c5c4c3)c3cccc4c3sc3ccccc34)c3c2oc2ccccc23)cc1]

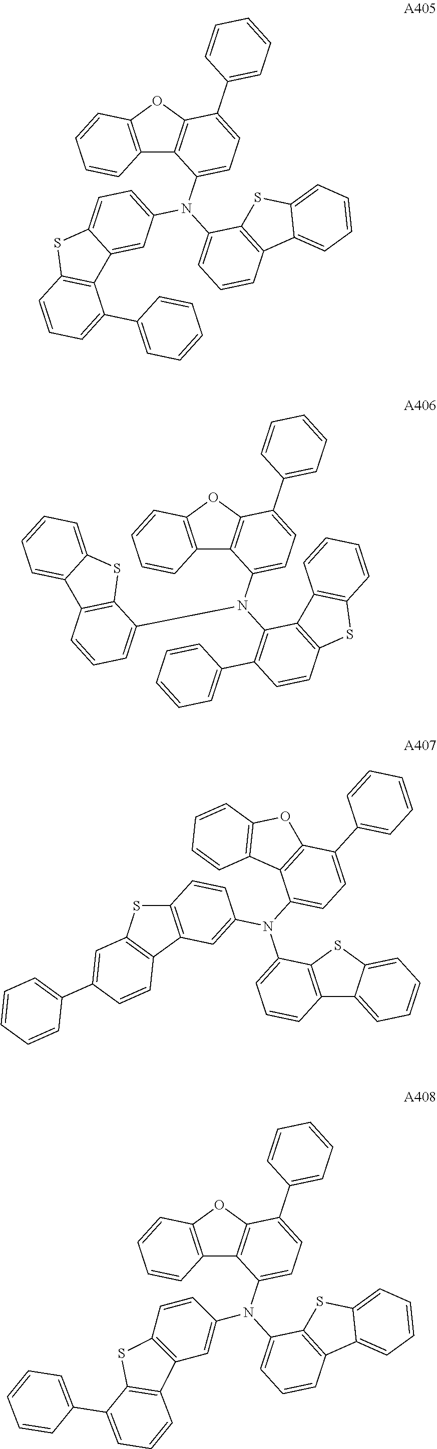 [SMILES: c1ccc(-c2ccc(N(c3ccc4sc5cccc(-c6ccccc6)c5c4c3)c3cccc4c3sc3ccccc34)c3c2oc2ccccc23)cc1 ; c1ccc(-c2ccc3sc4ccccc4c3c2N(c2cccc3c2sc2ccccc23)c2ccc(-c3ccccc3)c3oc4ccccc4c23)cc1 ; c1ccc(-c2ccc3c(c2)sc2ccc(N(c4cccc5c4sc4ccccc45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4c3sc3ccccc34)c3c2oc2ccccc23)cc1]

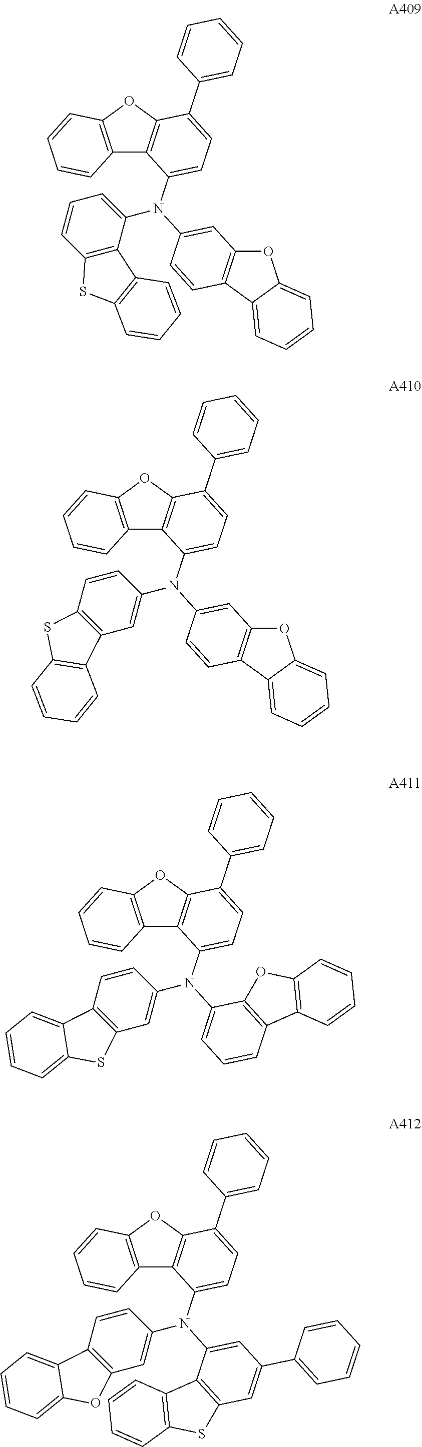 [SMILES: c1ccc(-c2ccc(N(c3ccc4c(c3)oc3ccccc34)c3cccc4sc5ccccc5c34)c3c2oc2ccccc23)cc1 ; c1ccc(-c2ccc(N(c3ccc4c(c3)oc3ccccc34)c3ccc4sc5ccccc5c4c3)c3c2oc2ccccc23)cc1 ; c1ccc(-c2ccc(N(c3ccc4c(c3)sc3ccccc34)c3cccc4c3oc3ccccc34)c3c2oc2ccccc23)cc1 ; c1ccc(-c2cc(N(c3ccc4c(c3)oc3ccccc34)c3ccc(-c4ccccc4)c4oc5ccccc5c34)c3c(c2)sc2ccccc23)cc1]

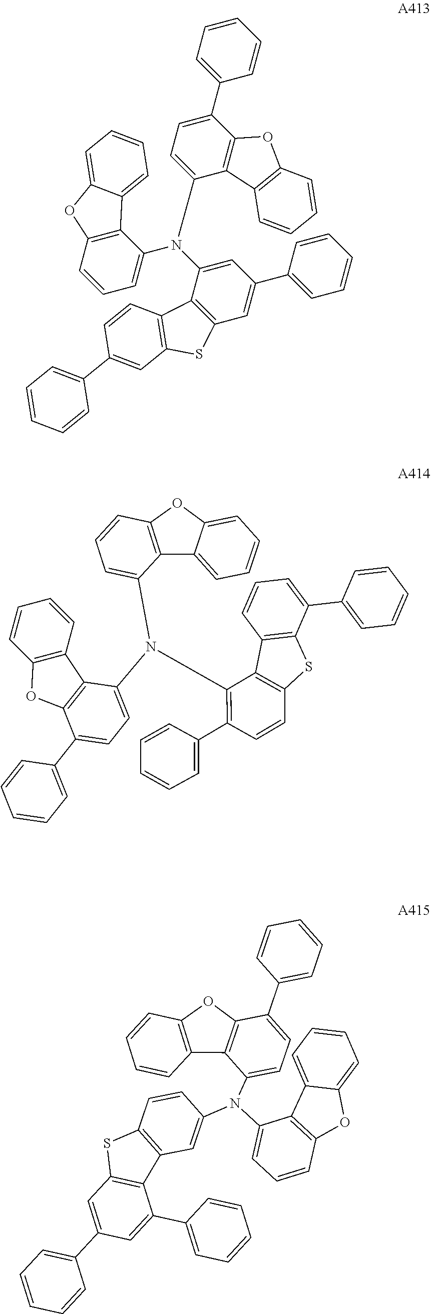 [SMILES: c1ccc(-c2ccc3c(c2)sc2cc(-c4ccccc4)cc(N(c4cccc5oc6ccccc6c45)c4ccc(-c5ccccc5)c5oc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3oc4ccccc4c23)cc1 ; c1ccc(-c2cc(-c3ccccc3)c3c(c2)sc2ccc(N(c4cccc5oc6ccccc6c45)c4ccc(-c5ccccc5)c5oc6ccccc6c45)cc23)cc1]

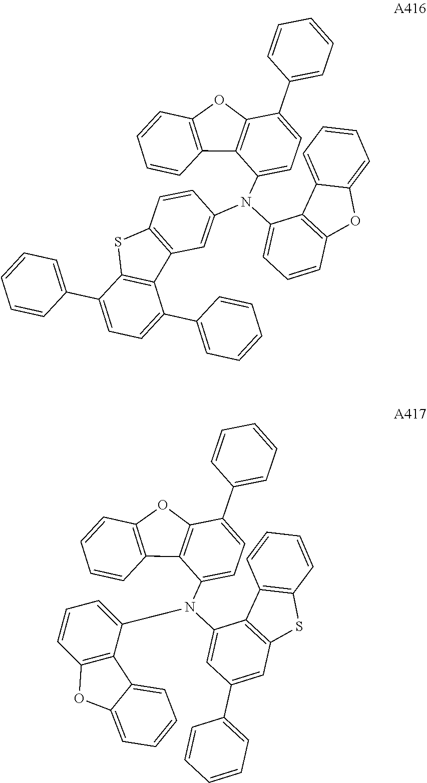 [SMILES: c1ccc(-c2ccc(N(c3ccc4sc5c(-c6ccccc6)ccc(-c6ccccc6)c5c4c3)c3cccc4oc5ccccc5c34)c3c2oc2ccccc23)cc1 ; c1ccc(-c2cc(N(c3cccc4oc5ccccc5c34)c3ccc(-c4ccccc4)c4oc5ccccc5c34)c3c(c2)sc2ccccc23)cc1]

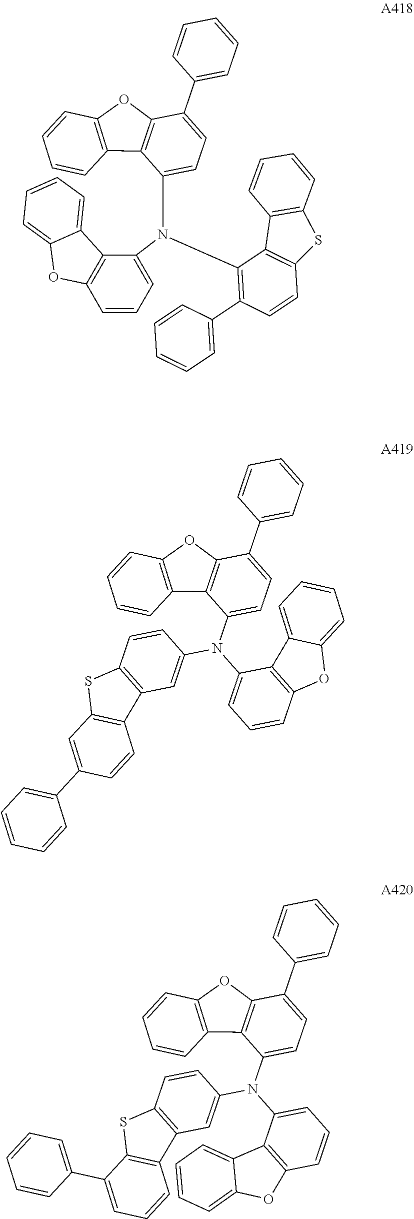 [SMILES: c1ccc(-c2ccc3sc4ccccc4c3c2N(c2cccc3oc4ccccc4c23)c2ccc(-c3ccccc3)c3oc4ccccc4c23)cc1 ; c1ccc(-c2ccc3c(c2)sc2ccc(N(c4cccc5oc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4oc5ccccc5c34)c3c2oc2ccccc23)cc1]

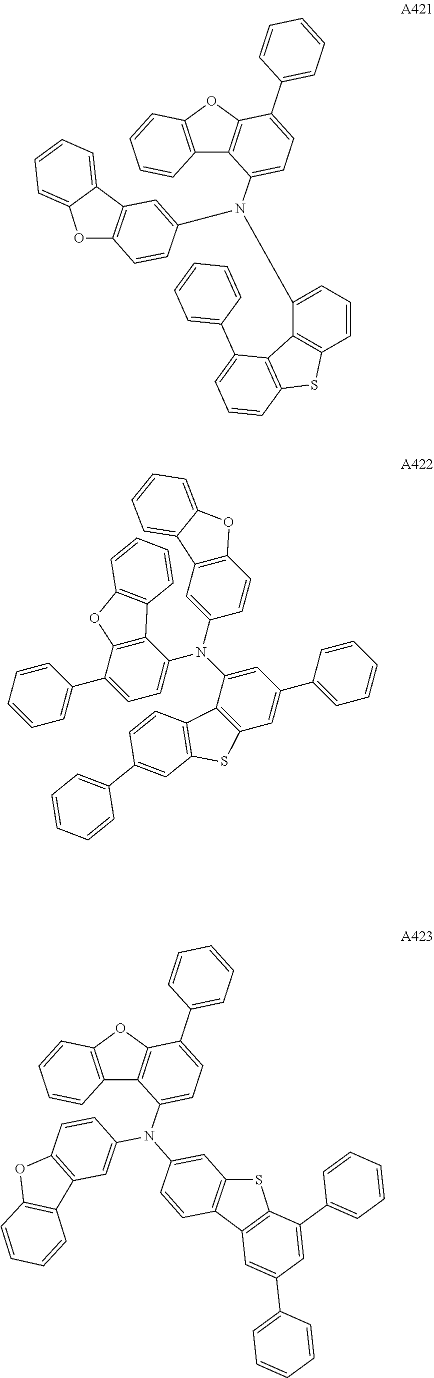 [SMILES: c1ccc(-c2ccc(N(c3ccc4oc5ccccc5c4c3)c3cccc4sc5cccc(-c6ccccc6)c5c34)c3c2oc2ccccc23)cc1 ; c1ccc(-c2ccc3c(c2)sc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3sc4cc(N(c5ccc6oc7ccccc7c6c5)c5ccc(-c6ccccc6)c6oc7ccccc7c56)ccc4c3c2)cc1]

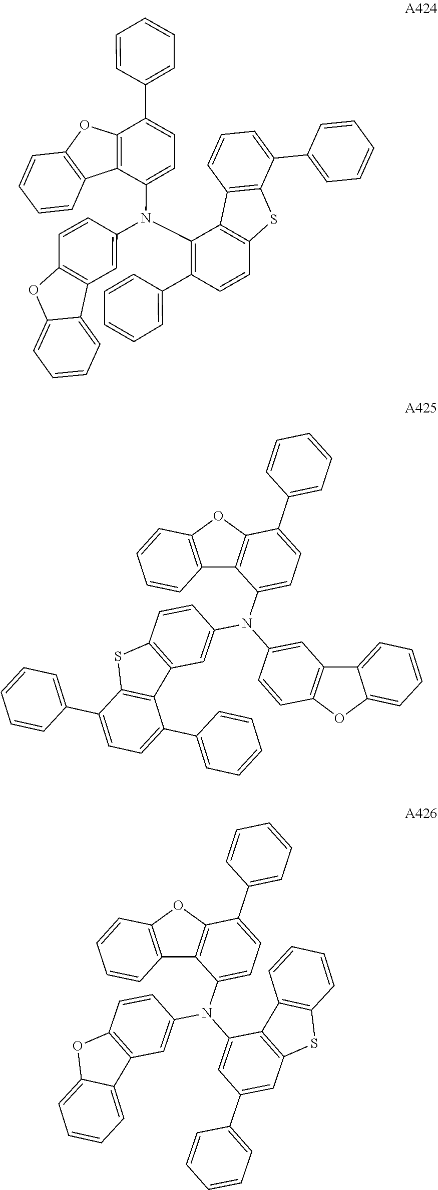 [SMILES: c1ccc(-c2ccc3sc4c(-c5ccccc5)cccc4c3c2N(c2ccc3oc4ccccc4c3c2)c2ccc(-c3ccccc3)c3oc4ccccc4c23)cc1 ; c1ccc(-c2ccc(N(c3ccc4oc5ccccc5c4c3)c3ccc4sc5c(-c6ccccc6)ccc(-c6ccccc6)c5c4c3)c3c2oc2ccccc23)cc1 ; c1ccc(-c2cc(N(c3ccc4oc5ccccc5c4c3)c3ccc(-c4ccccc4)c4oc5ccccc5c34)c3c(c2)sc2ccccc23)cc1]

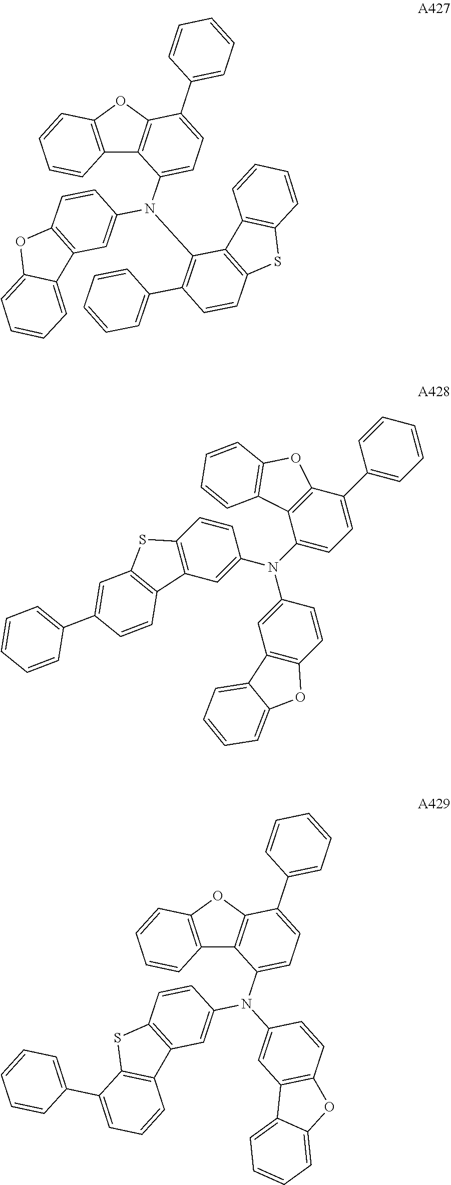 [SMILES: c1ccc(-c2ccc3sc4ccccc4c3c2N(c2ccc3oc4ccccc4c3c2)c2ccc(-c3ccccc3)c3oc4ccccc4c23)cc1 ; c1ccc(-c2ccc3c(c2)sc2ccc(N(c4ccc5oc6ccccc6c5c4)c4ccc(-c5ccccc5)c5oc6ccccc6c45)cc23)cc1 ; c1ccc(-c2ccc(N(c3ccc4oc5ccccc5c4c3)c3ccc4sc5c(-c6ccccc6)cccc5c4c3)c3c2oc2ccccc23)cc1]

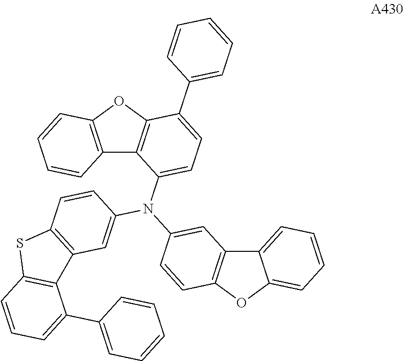 [SMILES: c1ccc(-c2ccc(N(c3ccc4oc5ccccc5c4c3)c3ccc4sc5cccc(-c6ccccc6)c5c4c3)c3c2oc2ccccc23)cc1]

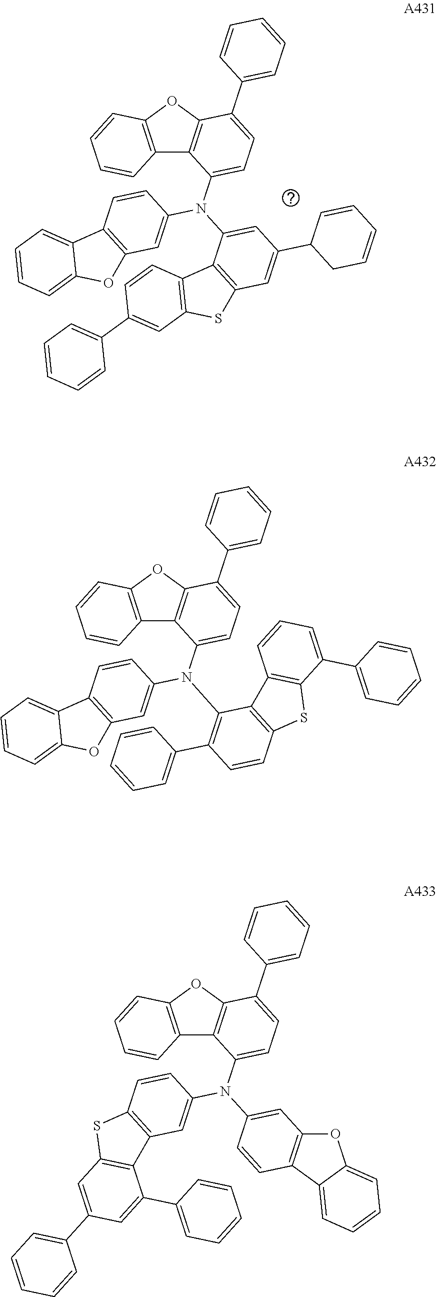 [SMILES: C1=CCC(c2cc(N(c3ccc4c(c3)oc3ccccc34)c3ccc(-c4ccccc4)c4oc5ccccc5c34)c3c(c2)sc2cc(-c4ccccc4)ccc23)C=C1 ; c1ccc(-c2ccc3sc4c(-c5ccccc5)cccc4c3c2N(c2ccc3c(c2)oc2ccccc23)c2ccc(-c3ccccc3)c3oc4ccccc4c23)cc1 ; c1ccc(-c2cc(-c3ccccc3)c3c(c2)sc2ccc(N(c4ccc5c(c4)oc4ccccc45)c4ccc(-c5ccccc5)c5oc6ccccc6c45)cc23)cc1]

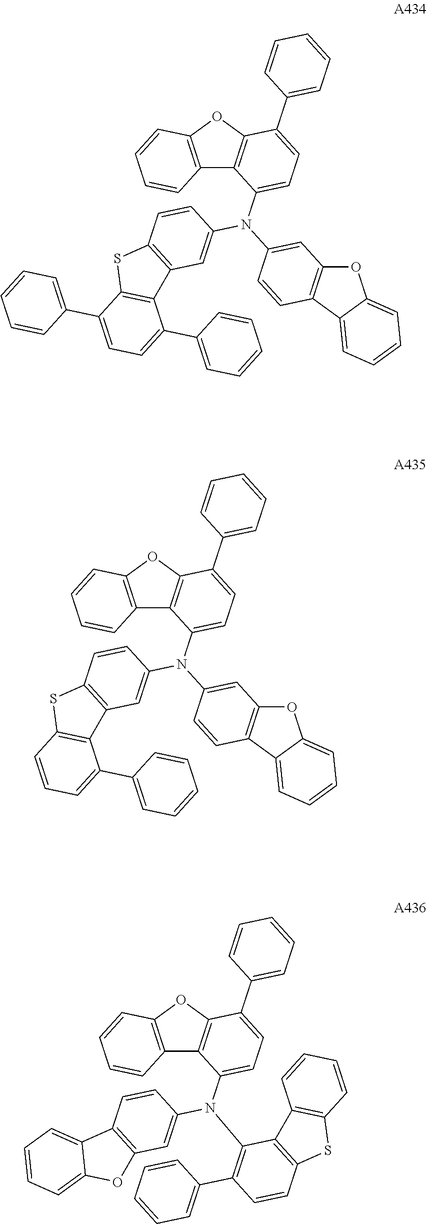 [SMILES: c1ccc(-c2ccc(N(c3ccc4c(c3)oc3ccccc34)c3ccc4sc5c(-c6ccccc6)ccc(-c6ccccc6)c5c4c3)c3c2oc2ccccc23)cc1 ; c1ccc(-c2ccc(N(c3ccc4c(c3)oc3ccccc34)c3ccc4sc5cccc(-c6ccccc6)c5c4c3)c3c2oc2ccccc23)cc1 ; c1ccc(-c2ccc3sc4ccccc4c3c2N(c2ccc3c(c2)oc2ccccc23)c2ccc(-c3ccccc3)c3oc4ccccc4c23)cc1]

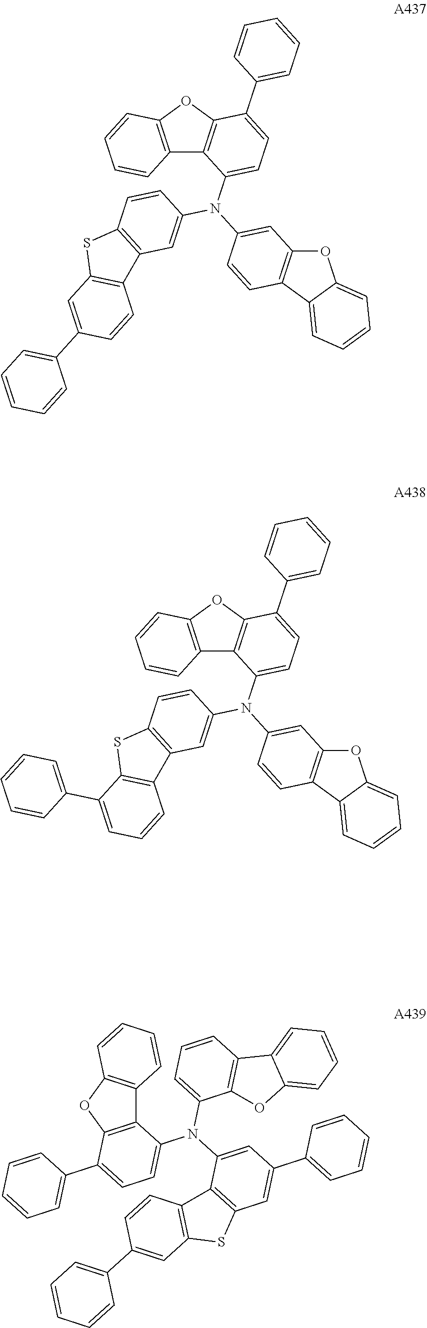 [SMILES: c1ccc(-c2ccc3c(c2)sc2ccc(N(c4ccc5c(c4)oc4ccccc45)c4ccc(-c5ccccc5)c5oc6ccccc6c45)cc23)cc1 ; c1ccc(-c2ccc(N(c3ccc4c(c3)oc3ccccc34)c3ccc4sc5c(-c6ccccc6)cccc5c4c3)c3c2oc2ccccc23)cc1 ; c1ccc(-c2ccc3c(c2)sc2cc(-c4ccccc4)cc(N(c4cccc5c4oc4ccccc45)c4ccc(-c5ccccc5)c5oc6ccccc6c45)c23)cc1]

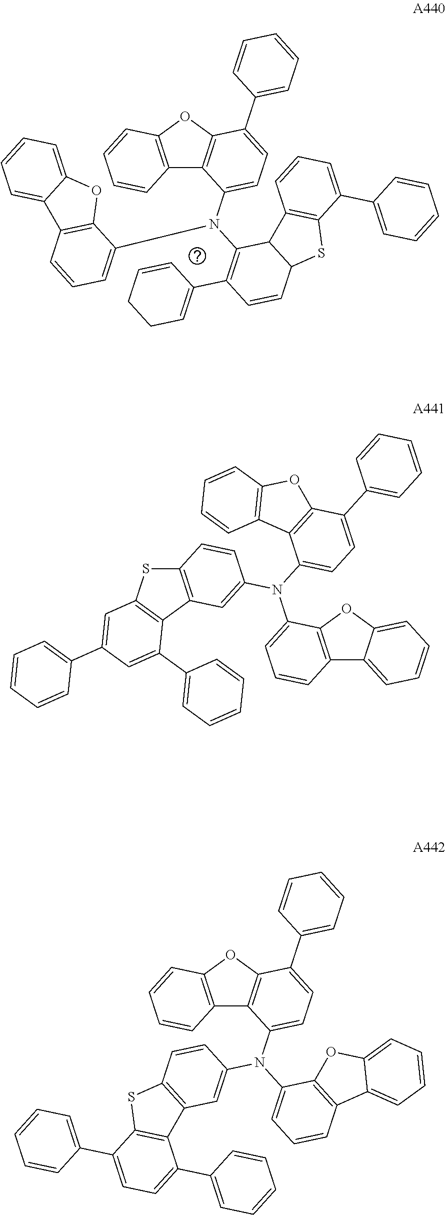 [SMILES: C1=CC(C2=C(N(c3cccc4c3oc3ccccc34)c3ccc(-c4ccccc4)c4oc5ccccc5c34)C3c4cccc(-c5ccccc5)c4SC3C=C2)=CCC1 ; c1ccc(-c2cc(-c3ccccc3)c3c(c2)sc2ccc(N(c4cccc5c4oc4ccccc45)c4ccc(-c5ccccc5)c5oc6ccccc6c45)cc23)cc1 ; c1ccc(-c2ccc(N(c3ccc4sc5c(-c6ccccc6)ccc(-c6ccccc6)c5c4c3)c3cccc4c3oc3ccccc34)c3c2oc2ccccc23)cc1]

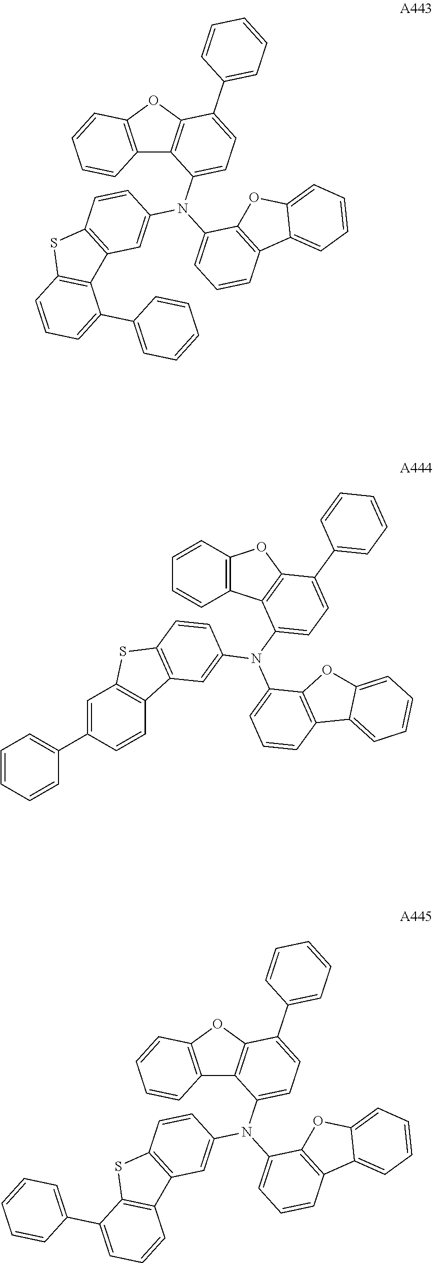 [SMILES: c1ccc(-c2ccc(N(c3ccc4sc5cccc(-c6ccccc6)c5c4c3)c3cccc4c3oc3ccccc34)c3c2oc2ccccc23)cc1 ; c1ccc(-c2ccc3c(c2)sc2ccc(N(c4cccc5c4oc4ccccc45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4c3oc3ccccc34)c3c2oc2ccccc23)cc1]

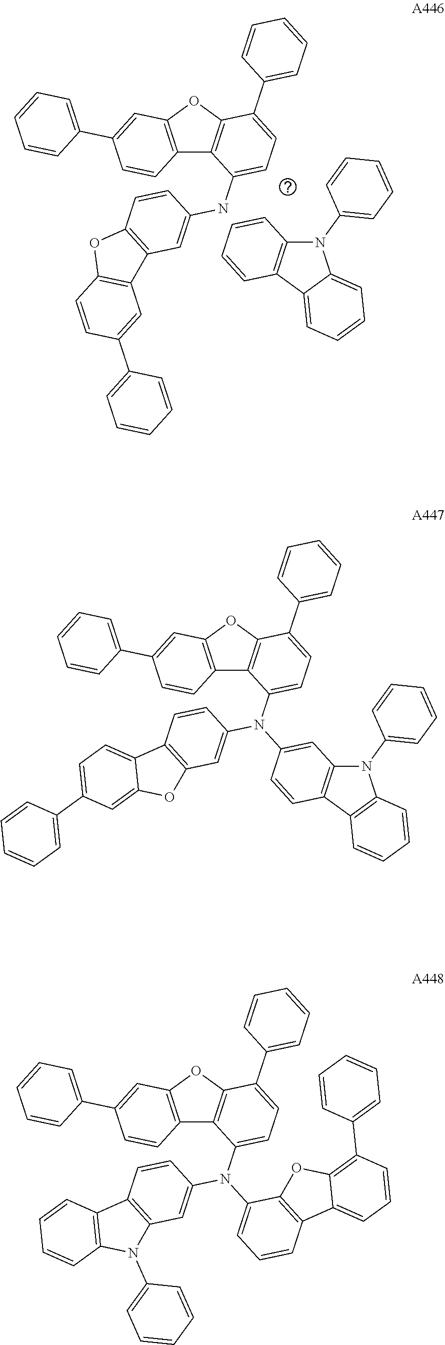

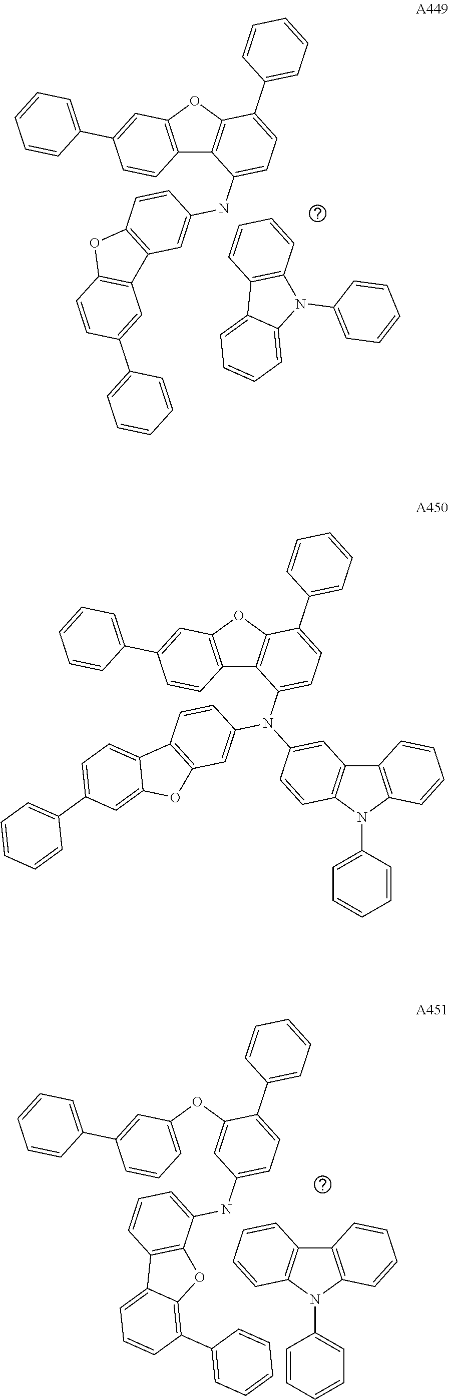

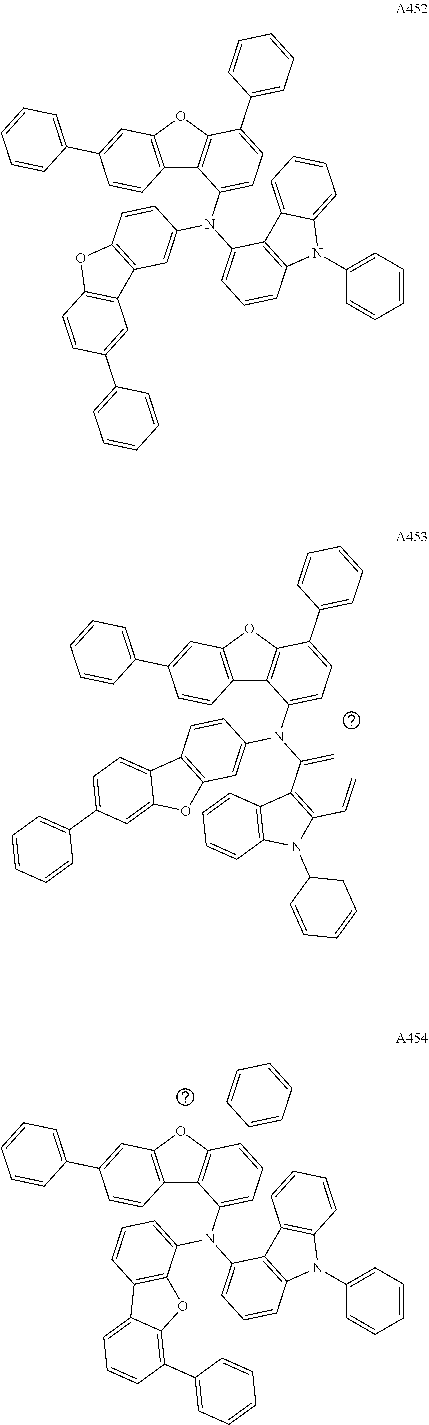

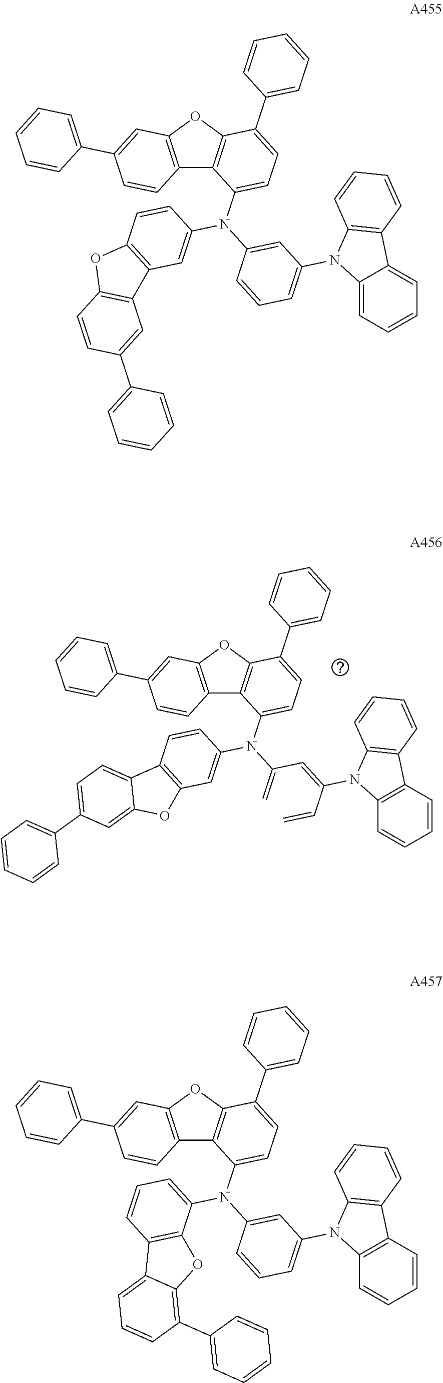 [SMILES: c1ccc(-c2ccc3c(c2)oc2c(-c4ccccc4)ccc(N(c4cccc(-n5c6ccccc6c6ccccc65)c4)c4ccc5oc6ccc(-c7ccccc7)cc6c5c4)c23)cc1 ; C=C/C(=C\C(=C)N(c1ccc2c(c1)oc1cc(-c3ccccc3)ccc12)c1ccc(-c2ccccc2)c2oc3cc(-c4ccccc4)ccc3c12)n1c2ccccc2c2ccccc21 ; c1ccc(-c2ccc3c(c2)oc2c(-c4ccccc4)ccc(N(c4cccc(-n5c6ccccc6c6ccccc65)c4)c4cccc5c4oc4c(-c6ccccc6)cccc45)c23)cc1]

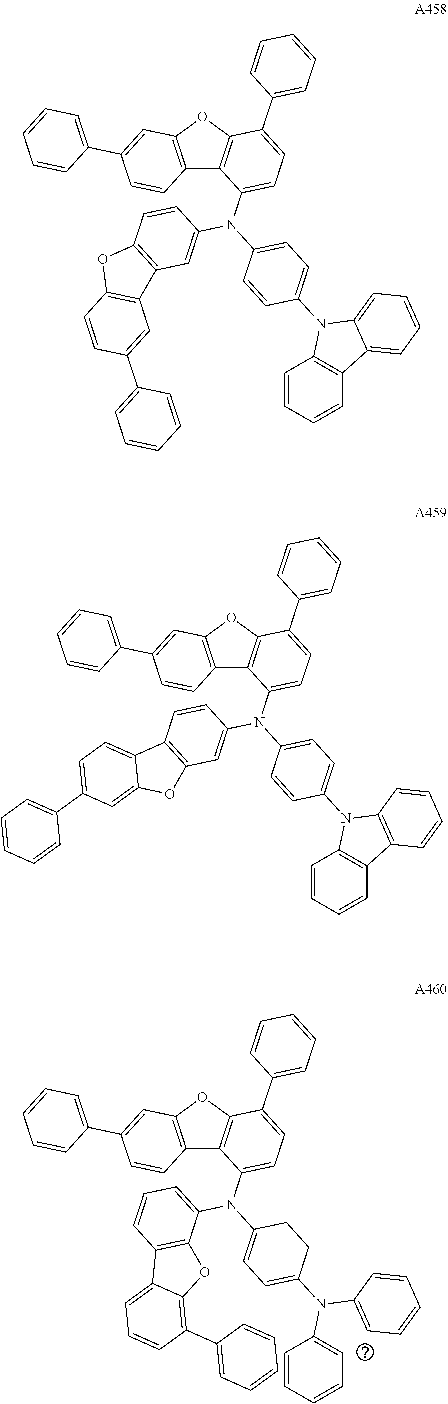 [SMILES: c1ccc(-c2ccc3c(c2)oc2c(-c4ccccc4)ccc(N(c4ccc(-n5c6ccccc6c6ccccc65)cc4)c4ccc5oc6ccc(-c7ccccc7)cc6c5c4)c23)cc1 ; c1ccc(-c2ccc3c(c2)oc2cc(N(c4ccc(-n5c6ccccc6c6ccccc65)cc4)c4ccc(-c5ccccc5)c5oc6cc(-c7ccccc7)ccc6c45)ccc23)cc1 ; C1=C(N(c2ccccc2)c2ccccc2)CCC(N(c2cccc3c2oc2c(-c4ccccc4)cccc23)c2ccc(-c3ccccc3)c3oc4cc(-c5ccccc5)ccc4c23)=C1]

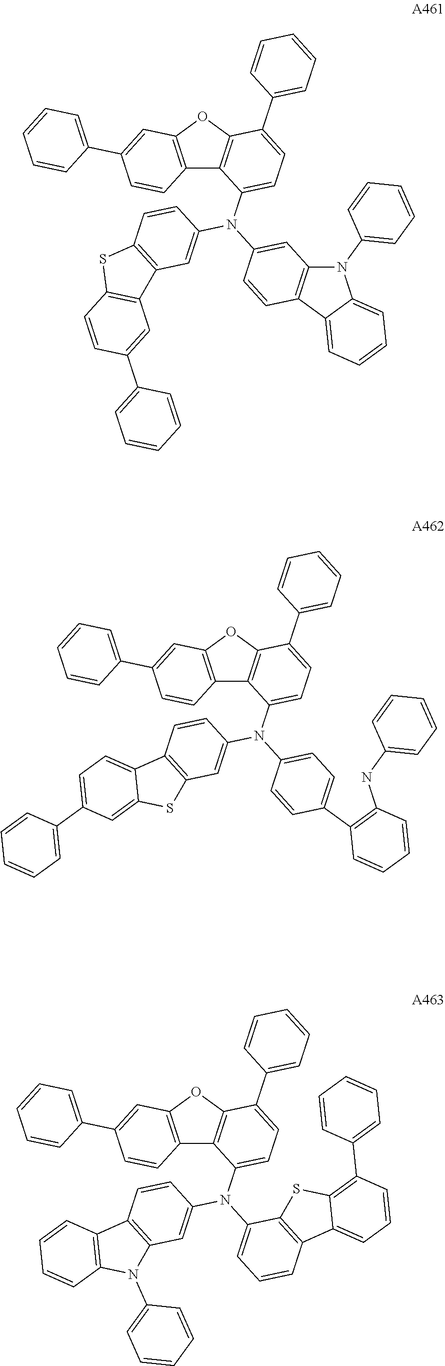 [SMILES: c1ccc(-c2ccc3c(c2)oc2c(-c4ccccc4)ccc(N(c4ccc5sc6ccc(-c7ccccc7)cc6c5c4)c4ccc5c6ccccc6n(-c6ccccc6)c5c4)c23)cc1 ; c1ccc(Nc2ccccc2-c2ccc(N(c3ccc4c(c3)sc3cc(-c5ccccc5)ccc34)c3ccc(-c4ccccc4)c4oc5cc(-c6ccccc6)ccc5c34)cc2)cc1 ; c1ccc(-c2ccc3c(c2)oc2c(-c4ccccc4)ccc(N(c4ccc5c6ccccc6n(-c6ccccc6)c5c4)c4cccc5c4sc4c(-c6ccccc6)cccc45)c23)cc1]

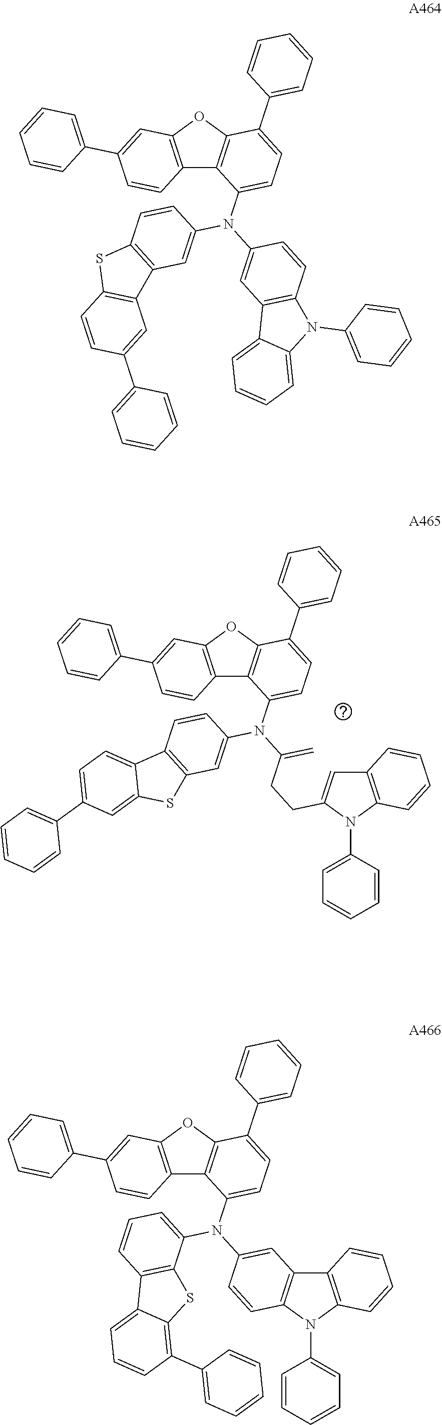 [SMILES: c1ccc(-c2ccc3c(c2)oc2c(-c4ccccc4)ccc(N(c4ccc5sc6ccc(-c7ccccc7)cc6c5c4)c4ccc5c(c4)c4ccccc4n5-c4ccccc4)c23)cc1 ; C=C(CCc1cc2ccccc2n1-c1ccccc1)N(c1ccc2c(c1)sc1cc(-c3ccccc3)ccc12)c1ccc(-c2ccccc2)c2oc3cc(-c4ccccc4)ccc3c12 ; c1ccc(-c2ccc3c(c2)oc2c(-c4ccccc4)ccc(N(c4ccc5c(c4)c4ccccc4n5-c4ccccc4)c4cccc5c4sc4c(-c6ccccc6)cccc45)c23)cc1]

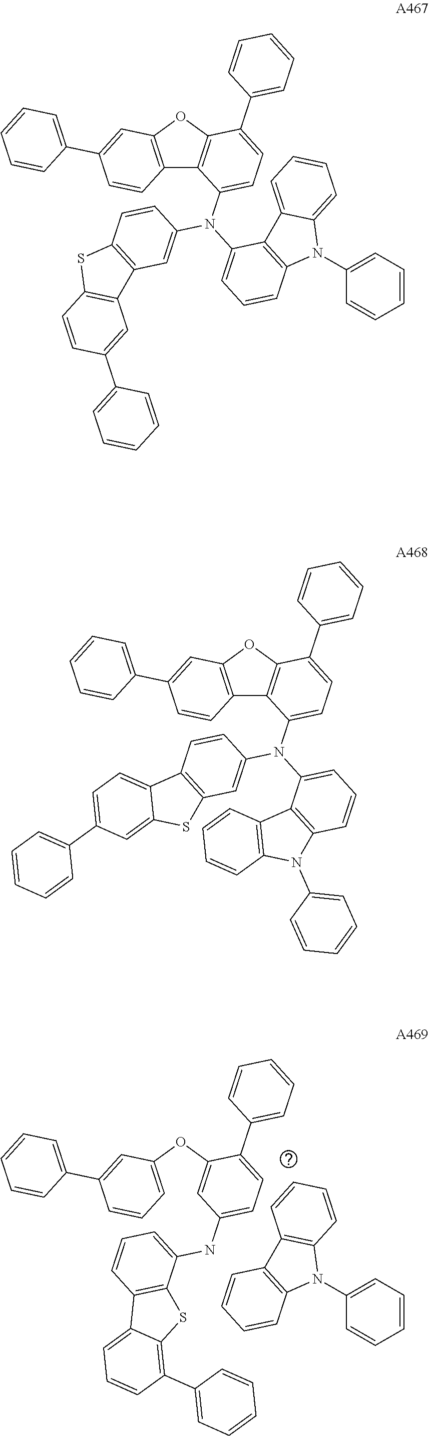

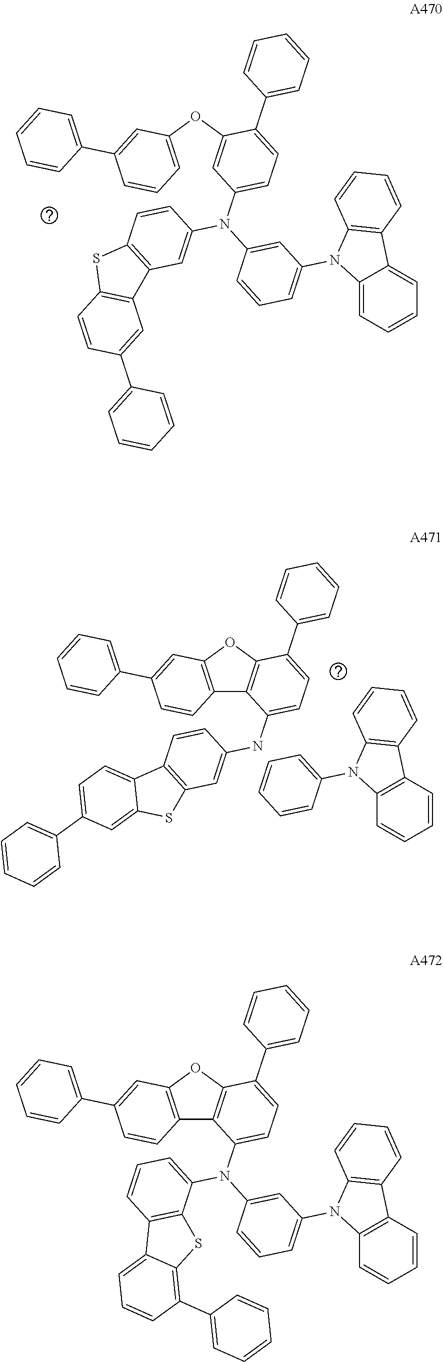

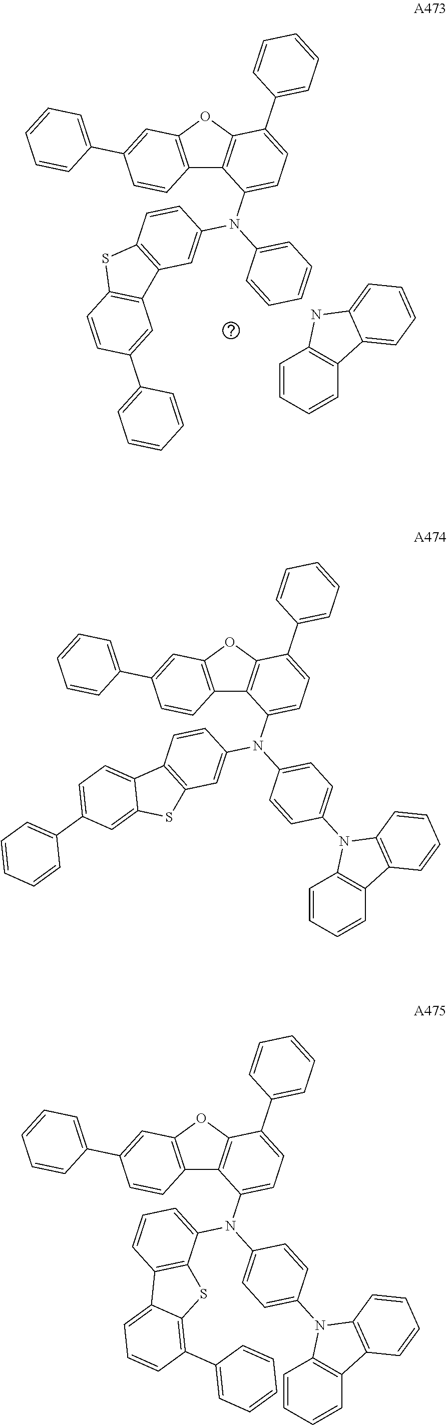

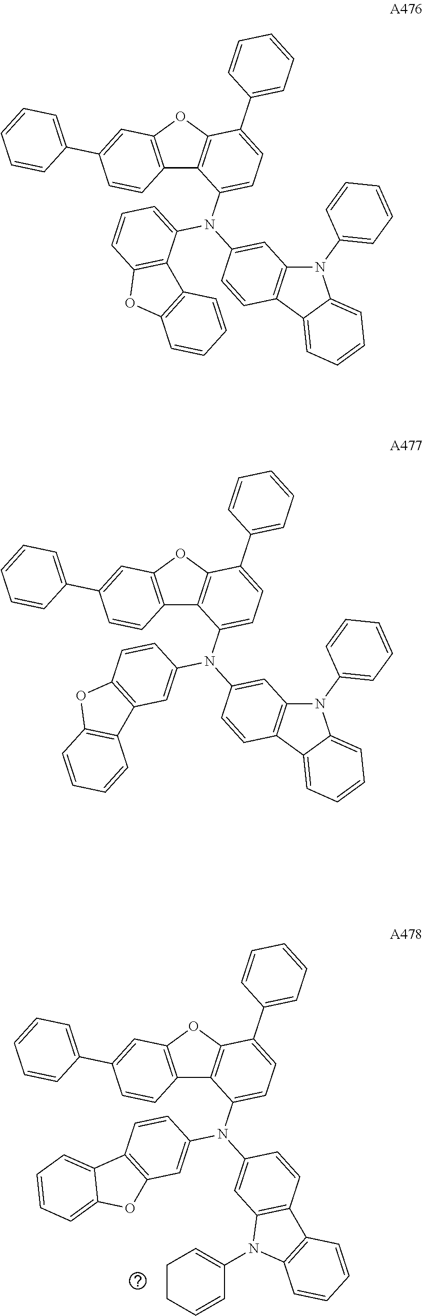 [SMILES: c1ccc(-c2ccc3c(c2)oc2c(-c4ccccc4)ccc(N(c4ccc5c6ccccc6n(-c6ccccc6)c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4ccc5c6ccccc6n(-c6ccccc6)c5c4)c23)cc1 ; C1=CC(n2c3ccccc3c3ccc(N(c4ccc5c(c4)oc4ccccc45)c4ccc(-c5ccccc5)c5oc6cc(-c7ccccc7)ccc6c45)cc32)=CCC1]

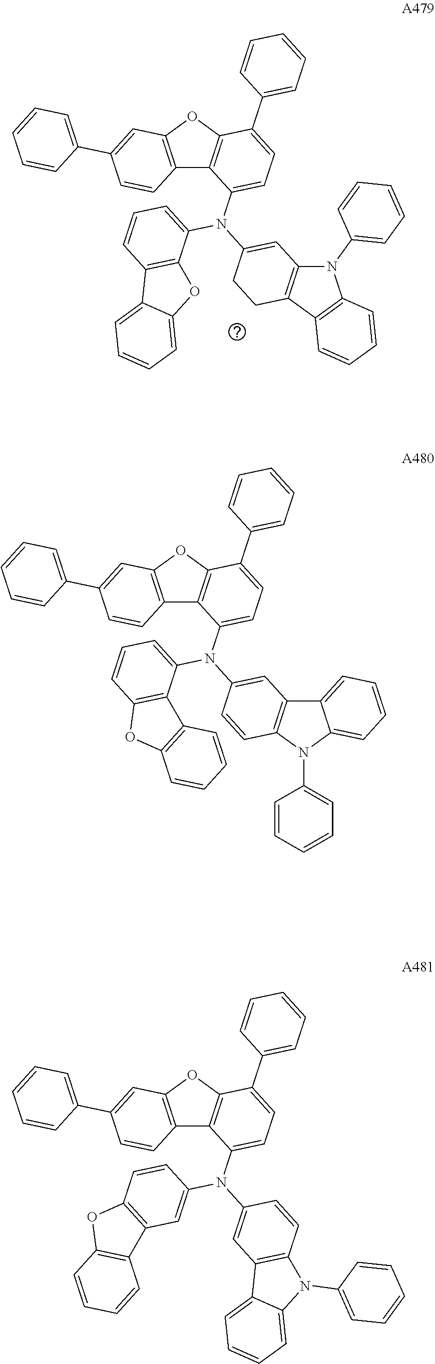 [SMILES: C1=C(N(c2cccc3c2oc2ccccc23)c2ccc(-c3ccccc3)c3oc4cc(-c5ccccc5)ccc4c23)CCc2c1n(-c1ccccc1)c1ccccc21 ; c1ccc(-c2ccc3c(c2)oc2c(-c4ccccc4)ccc(N(c4ccc5c(c4)c4ccccc4n5-c4ccccc4)c4cccc5oc6ccccc6c45)c23)cc1 ; c1ccc(-c2ccc3c(c2)oc2c(-c4ccccc4)ccc(N(c4ccc5oc6ccccc6c5c4)c4ccc5c(c4)c4ccccc4n5-c4ccccc4)c23)cc1]

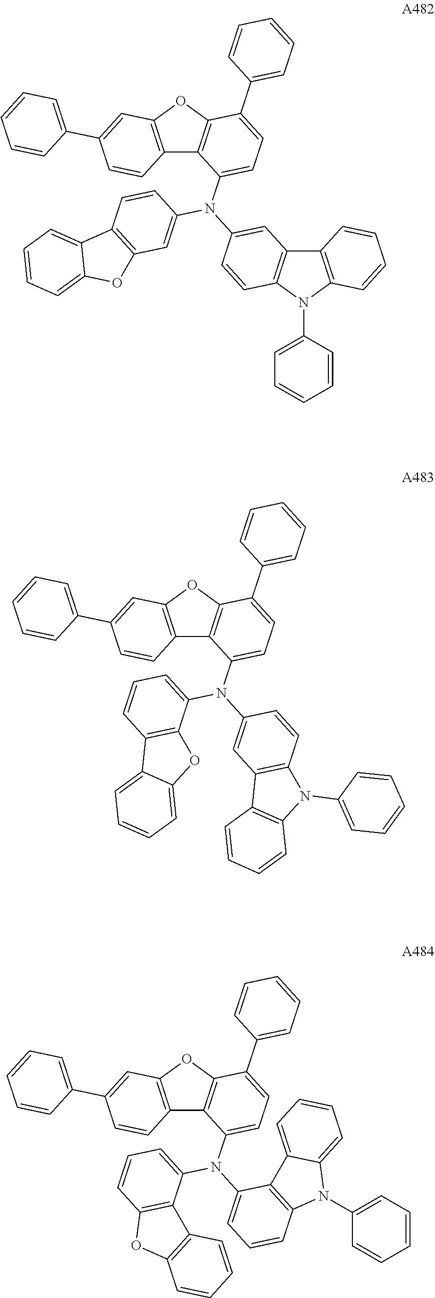 [SMILES: c1ccc(-c2ccc3c(c2)oc2c(-c4ccccc4)ccc(N(c4ccc5c(c4)oc4ccccc45)c4ccc5c(c4)c4ccccc4n5-c4ccccc4)c23)cc1 ; c1ccc(-c2ccc3c(c2)oc2c(-c4ccccc4)ccc(N(c4ccc5c(c4)c4ccccc4n5-c4ccccc4)c4cccc5c4oc4ccccc45)c23)cc1 ; c1ccc(-c2ccc3c(c2)oc2c(-c4ccccc4)ccc(N(c4cccc5oc6ccccc6c45)c4cccc5c4c4ccccc4n5-c4ccccc4)c23)cc1]

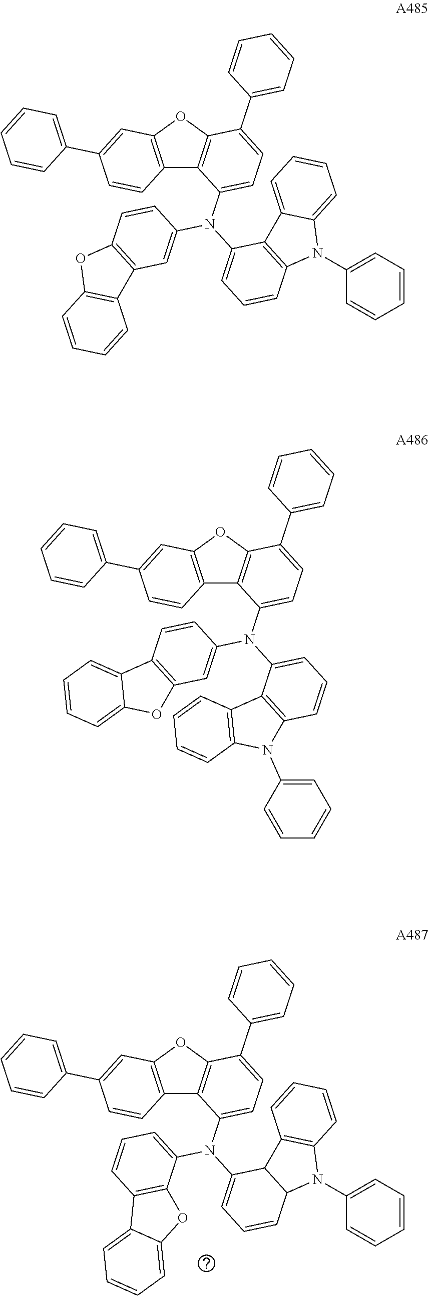 [SMILES: c1ccc(-c2ccc3c(c2)oc2c(-c4ccccc4)ccc(N(c4ccc5oc6ccccc6c5c4)c4cccc5c4c4ccccc4n5-c4ccccc4)c23)cc1 ; c1ccc(-c2ccc3c(c2)oc2c(-c4ccccc4)ccc(N(c4ccc5c(c4)oc4ccccc45)c4cccc5c4c4ccccc4n5-c4ccccc4)c23)cc1 ; C1=CC2C(C(N(c3cccc4c3oc3ccccc34)c3ccc(-c4ccccc4)c4oc5cc(-c6ccccc6)ccc5c34)=C1)c1ccccc1N2c1ccccc1]

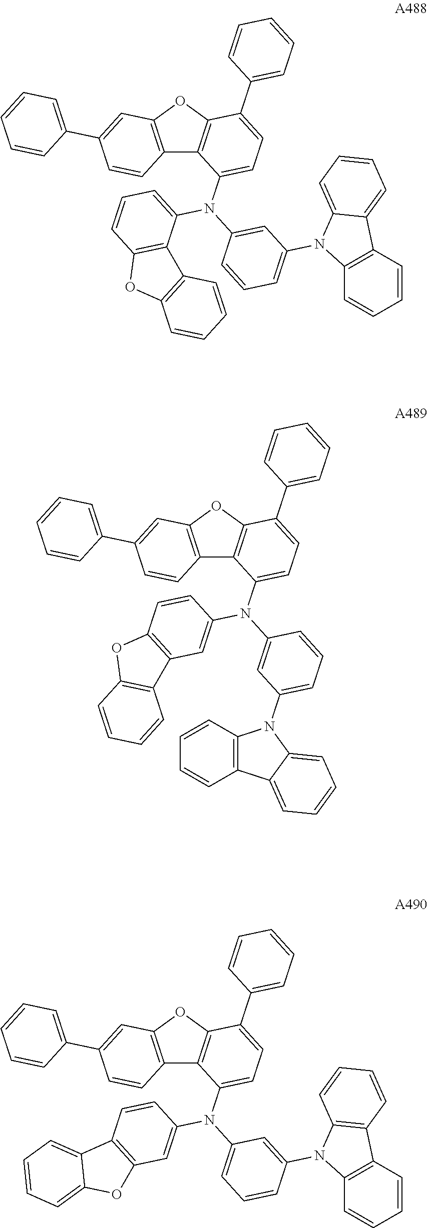 [SMILES: c1ccc(-c2ccc3c(c2)oc2c(-c4ccccc4)ccc(N(c4cccc(-n5c6ccccc6c6ccccc65)c4)c4cccc5oc6ccccc6c45)c23)cc1 ; c1ccc(-c2ccc3c(c2)oc2c(-c4ccccc4)ccc(N(c4cccc(-n5c6ccccc6c6ccccc65)c4)c4ccc5oc6ccccc6c5c4)c23)cc1 ; c1ccc(-c2ccc3c(c2)oc2c(-c4ccccc4)ccc(N(c4cccc(-n5c6ccccc6c6ccccc65)c4)c4ccc5c(c4)oc4ccccc45)c23)cc1]

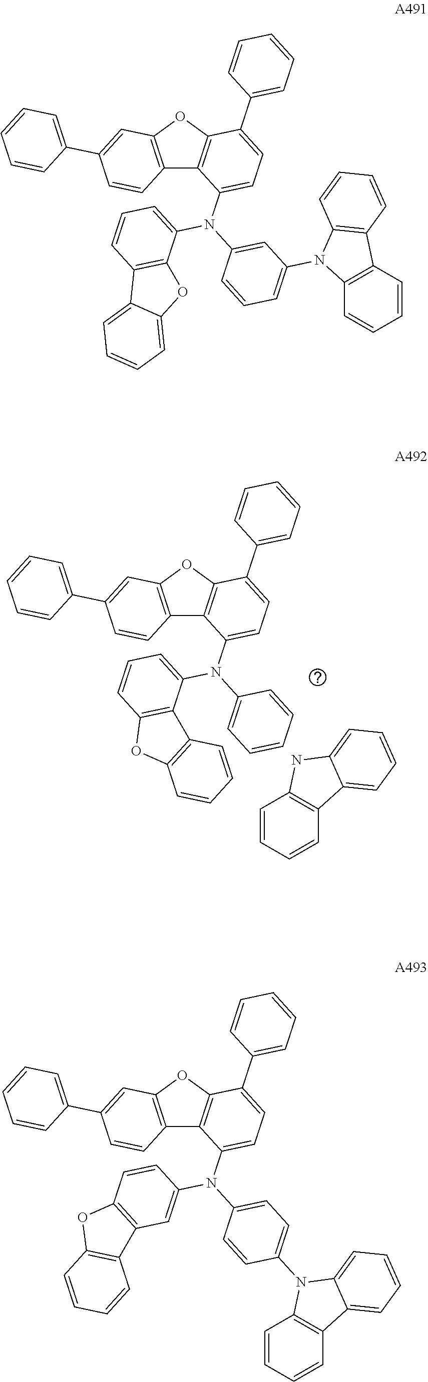

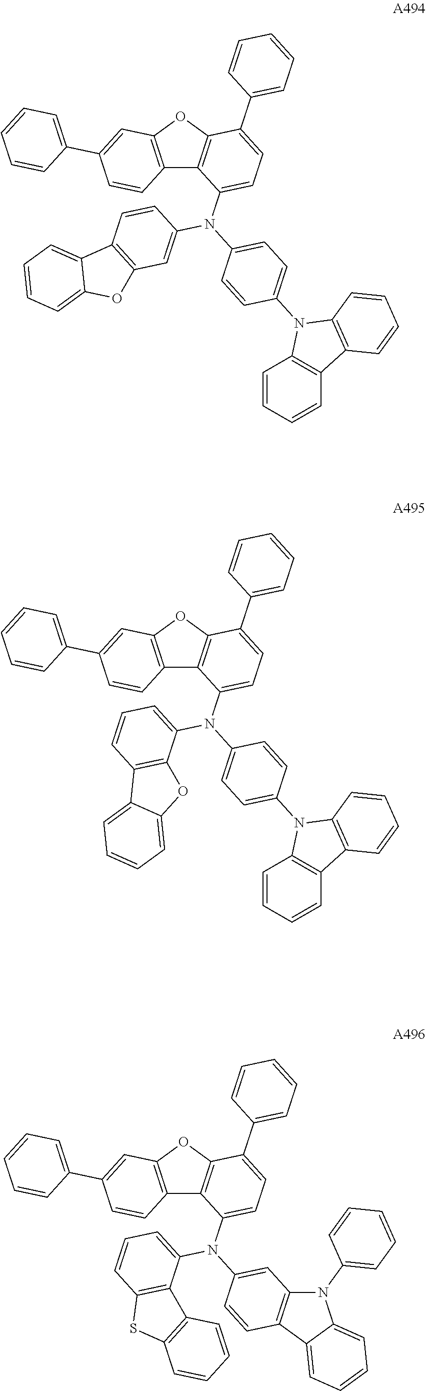 [SMILES: c1ccc(-c2ccc3c(c2)oc2c(-c4ccccc4)ccc(N(c4ccc(-n5c6ccccc6c6ccccc65)cc4)c4ccc5c(c4)oc4ccccc45)c23)cc1 ; c1ccc(-c2ccc3c(c2)oc2c(-c4ccccc4)ccc(N(c4ccc(-n5c6ccccc6c6ccccc65)cc4)c4cccc5c4oc4ccccc45)c23)cc1 ; c1ccc(-c2ccc3c(c2)oc2c(-c4ccccc4)ccc(N(c4ccc5c6ccccc6n(-c6ccccc6)c5c4)c4cccc5sc6ccccc6c45)c23)cc1]

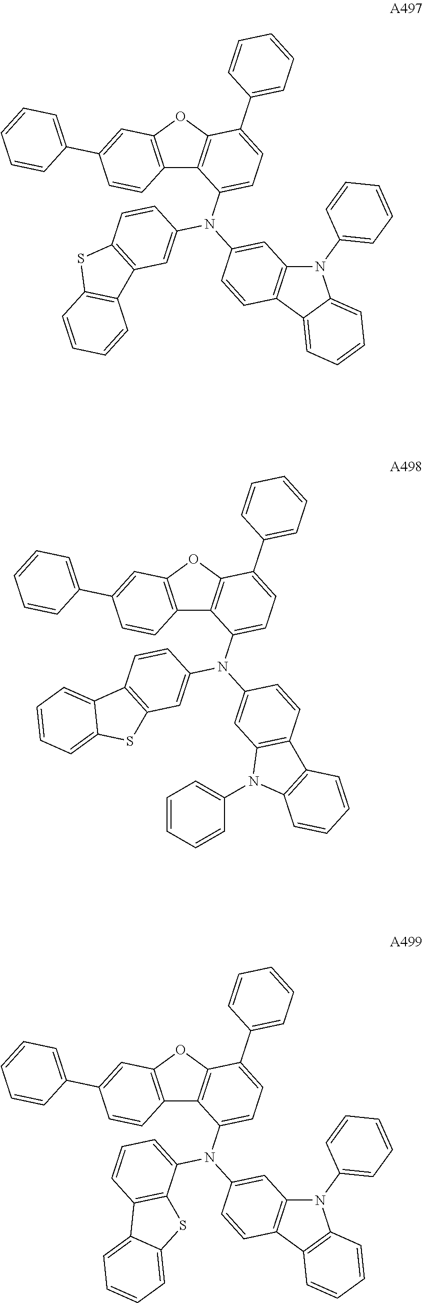 [SMILES: c1ccc(-c2ccc3c(c2)oc2c(-c4ccccc4)ccc(N(c4ccc5sc6ccccc6c5c4)c4ccc5c6ccccc6n(-c6ccccc6)c5c4)c23)cc1 ; c1ccc(-c2ccc3c(c2)oc2c(-c4ccccc4)ccc(N(c4ccc5c(c4)sc4ccccc45)c4ccc5c6ccccc6n(-c6ccccc6)c5c4)c23)cc1 ; c1ccc(-c2ccc3c(c2)oc2c(-c4ccccc4)ccc(N(c4ccc5c6ccccc6n(-c6ccccc6)c5c4)c4cccc5c4sc4ccccc45)c23)cc1]

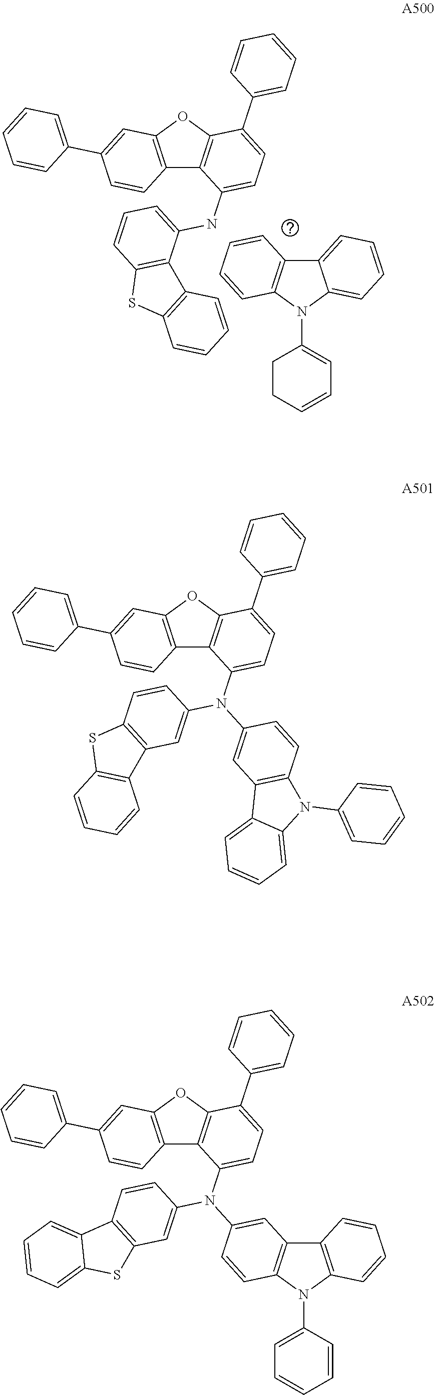

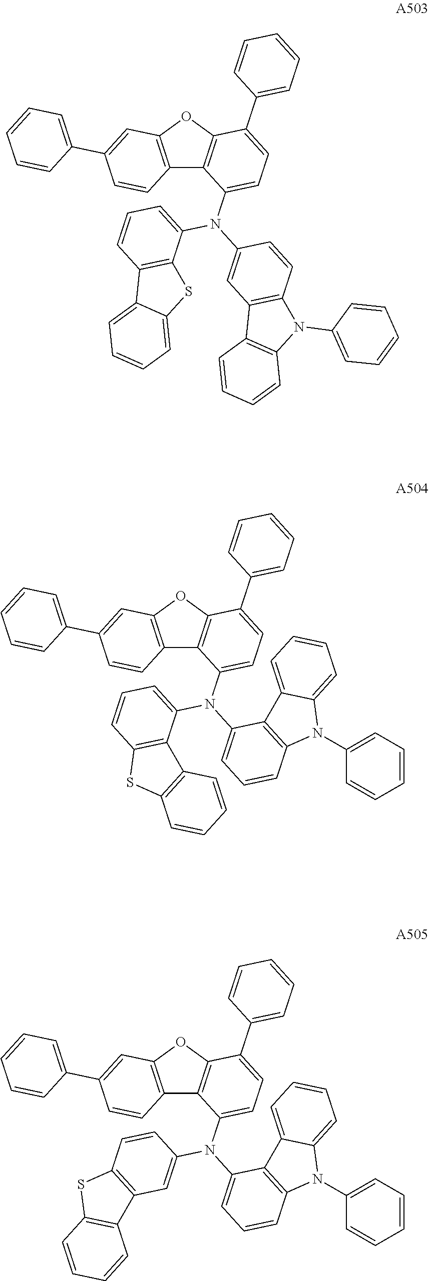 [SMILES: c1ccc(-c2ccc3c(c2)oc2c(-c4ccccc4)ccc(N(c4ccc5c(c4)c4ccccc4n5-c4ccccc4)c4cccc5c4sc4ccccc45)c23)cc1 ; c1ccc(-c2ccc3c(c2)oc2c(-c4ccccc4)ccc(N(c4cccc5sc6ccccc6c45)c4cccc5c4c4ccccc4n5-c4ccccc4)c23)cc1 ; c1ccc(-c2ccc3c(c2)oc2c(-c4ccccc4)ccc(N(c4ccc5sc6ccccc6c5c4)c4cccc5c4c4ccccc4n5-c4ccccc4)c23)cc1]

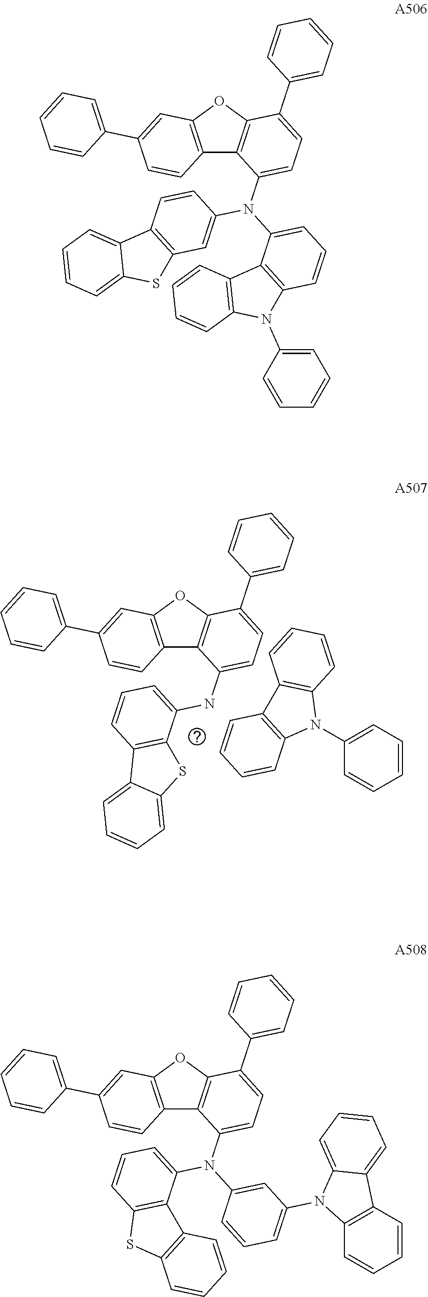

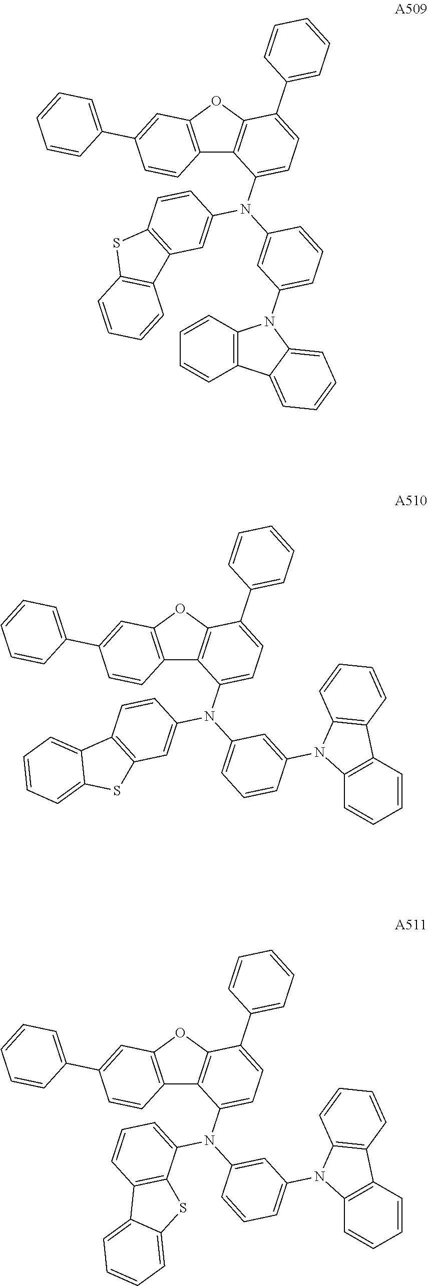 [SMILES: c1ccc(-c2ccc3c(c2)oc2c(-c4ccccc4)ccc(N(c4cccc(-n5c6ccccc6c6ccccc65)c4)c4ccc5sc6ccccc6c5c4)c23)cc1 ; c1ccc(-c2ccc3c(c2)oc2c(-c4ccccc4)ccc(N(c4cccc(-n5c6ccccc6c6ccccc65)c4)c4ccc5c(c4)sc4ccccc45)c23)cc1 ; c1ccc(-c2ccc3c(c2)oc2c(-c4ccccc4)ccc(N(c4cccc(-n5c6ccccc6c6ccccc65)c4)c4cccc5c4sc4ccccc45)c23)cc1]

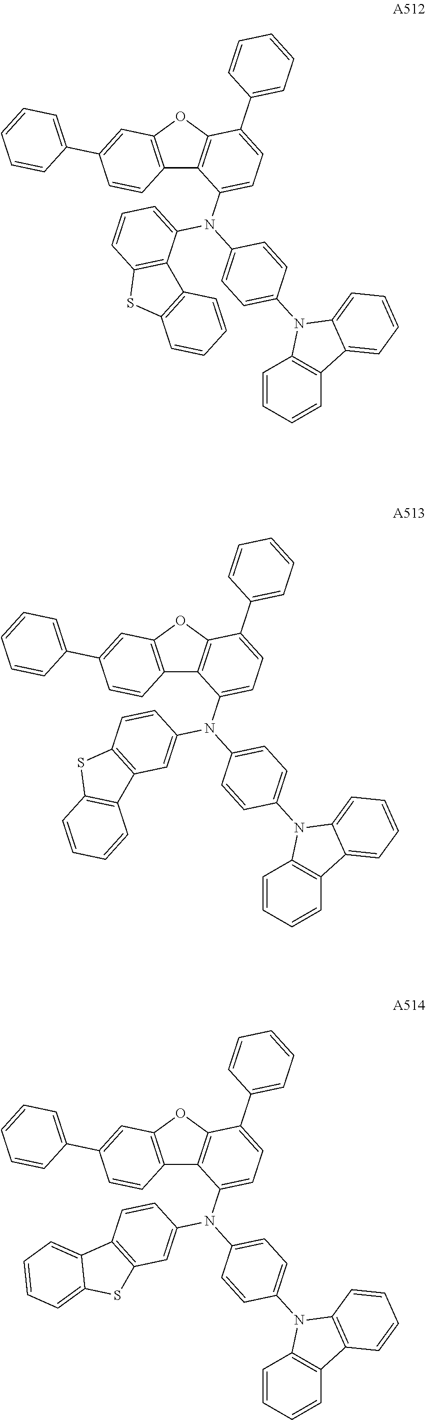 [SMILES: c1ccc(-c2ccc3c(c2)oc2c(-c4ccccc4)ccc(N(c4ccc(-n5c6ccccc6c6ccccc65)cc4)c4cccc5sc6ccccc6c45)c23)cc1 ; c1ccc(-c2ccc3c(c2)oc2c(-c4ccccc4)ccc(N(c4ccc(-n5c6ccccc6c6ccccc65)cc4)c4ccc5sc6ccccc6c5c4)c23)cc1 ; c1ccc(-c2ccc3c(c2)oc2c(-c4ccccc4)ccc(N(c4ccc(-n5c6ccccc6c6ccccc65)cc4)c4ccc5c(c4)sc4ccccc45)c23)cc1]

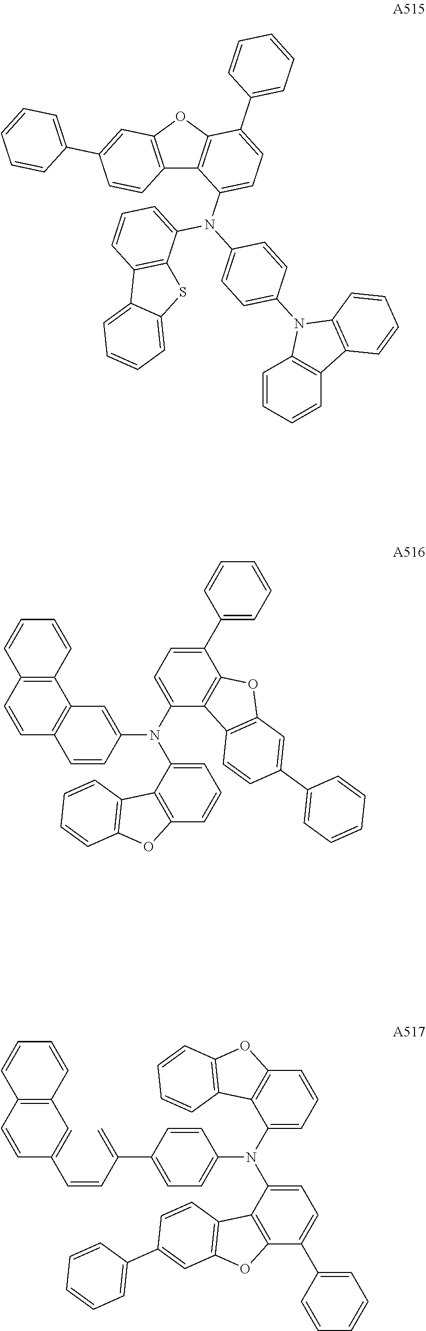 [SMILES: c1ccc(-c2ccc3c(c2)oc2c(-c4ccccc4)ccc(N(c4ccc(-n5c6ccccc6c6ccccc65)cc4)c4cccc5c4sc4ccccc45)c23)cc1 ; c1ccc(-c2ccc3c(c2)oc2c(-c4ccccc4)ccc(N(c4ccc5ccc6ccccc6c5c4)c4cccc5oc6ccccc6c45)c23)cc1 ; C=C(/C=C\c1ccc2ccccc2c1)c1ccc(N(c2cccc3oc4ccccc4c23)c2ccc(-c3ccccc3)c3oc4cc(-c5ccccc5)ccc4c23)cc1]

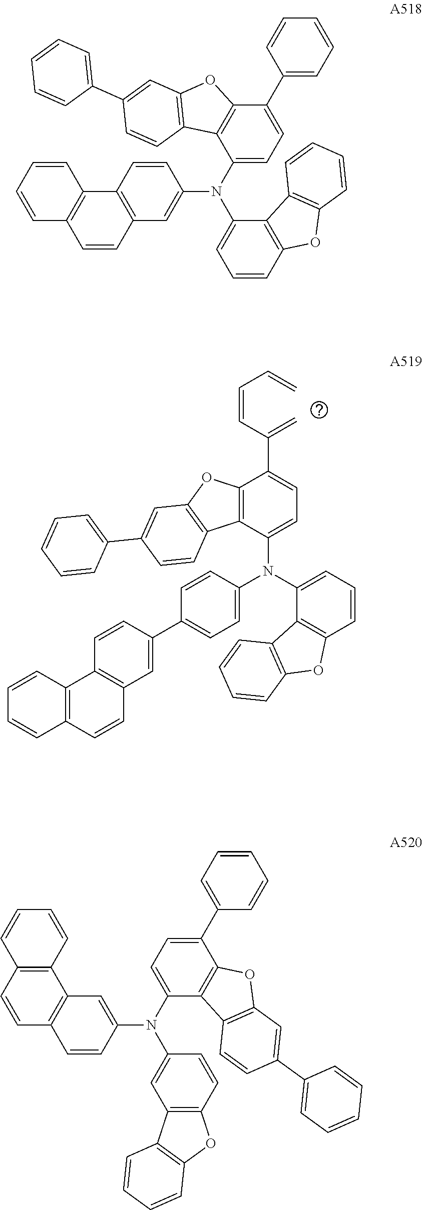 [SMILES: c1ccc(-c2ccc3c(c2)oc2c(-c4ccccc4)ccc(N(c4ccc5c(ccc6ccccc65)c4)c4cccc5oc6ccccc6c45)c23)cc1 ; C=C/C=C\C(=C)c1ccc(N(c2ccc(-c3ccc4c(ccc5ccccc54)c3)cc2)c2cccc3oc4ccccc4c23)c2c1oc1cc(-c3ccccc3)ccc12 ; c1ccc(-c2ccc3c(c2)oc2c(-c4ccccc4)ccc(N(c4ccc5ccc6ccccc6c5c4)c4ccc5oc6ccccc6c5c4)c23)cc1]

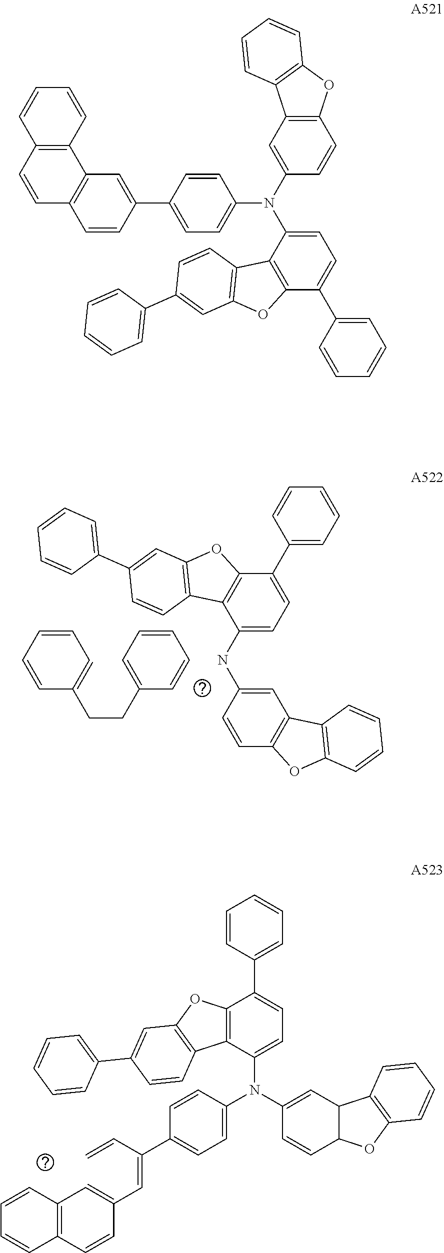

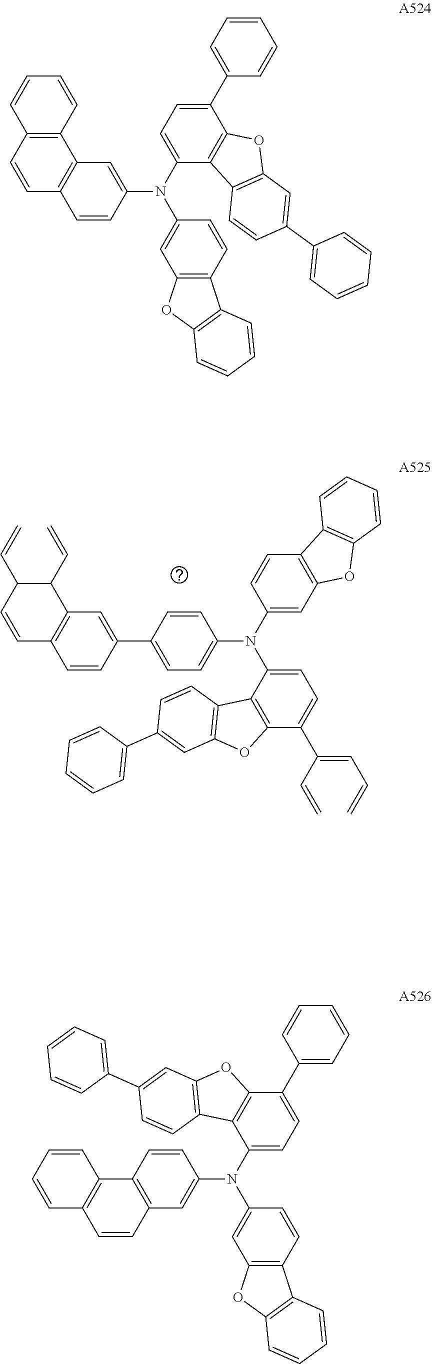 [SMILES: c1ccc(-c2ccc3c(c2)oc2c(-c4ccccc4)ccc(N(c4ccc5c(c4)oc4ccccc45)c4ccc5ccc6ccccc6c5c4)c23)cc1 ; C=C/C=C(\C=C)c1ccc(N(c2ccc(-c3ccc4c(c3)C(C=C)C(C=C)C=C4)cc2)c2ccc3c(c2)oc2ccccc23)c2c1oc1cc(-c3ccccc3)ccc12 ; c1ccc(-c2ccc3c(c2)oc2c(-c4ccccc4)ccc(N(c4ccc5c(ccc6ccccc65)c4)c4ccc5c(c4)oc4ccccc45)c23)cc1]

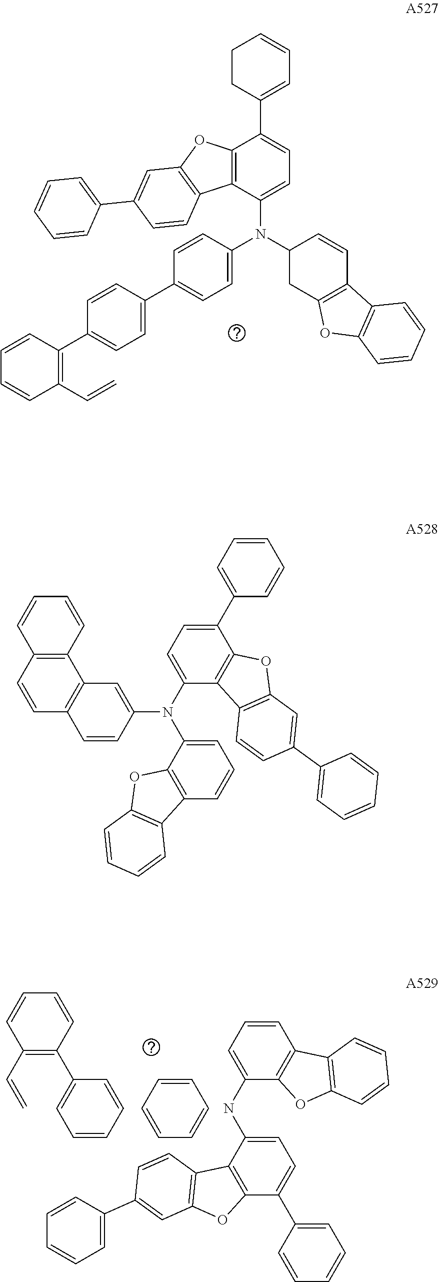

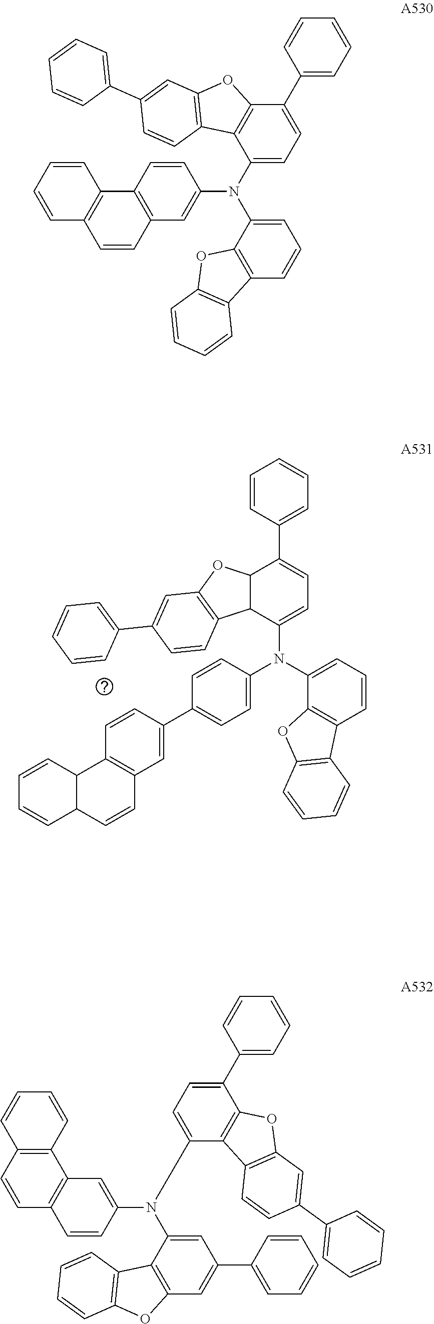 [SMILES: c1ccc(-c2ccc3c(c2)oc2c(-c4ccccc4)ccc(N(c4ccc5c(ccc6ccccc65)c4)c4cccc5c4oc4ccccc45)c23)cc1 ; C1=CC2C=Cc3cc(-c4ccc(N(C5=CC=C(c6ccccc6)C6Oc7cc(-c8ccccc8)ccc7C56)c5cccc6c5oc5ccccc56)cc4)ccc3C2C=C1 ; c1ccc(-c2ccc3c(c2)oc2c(-c4ccccc4)ccc(N(c4ccc5ccc6ccccc6c5c4)c4cc(-c5ccccc5)cc5oc6ccccc6c45)c23)cc1]

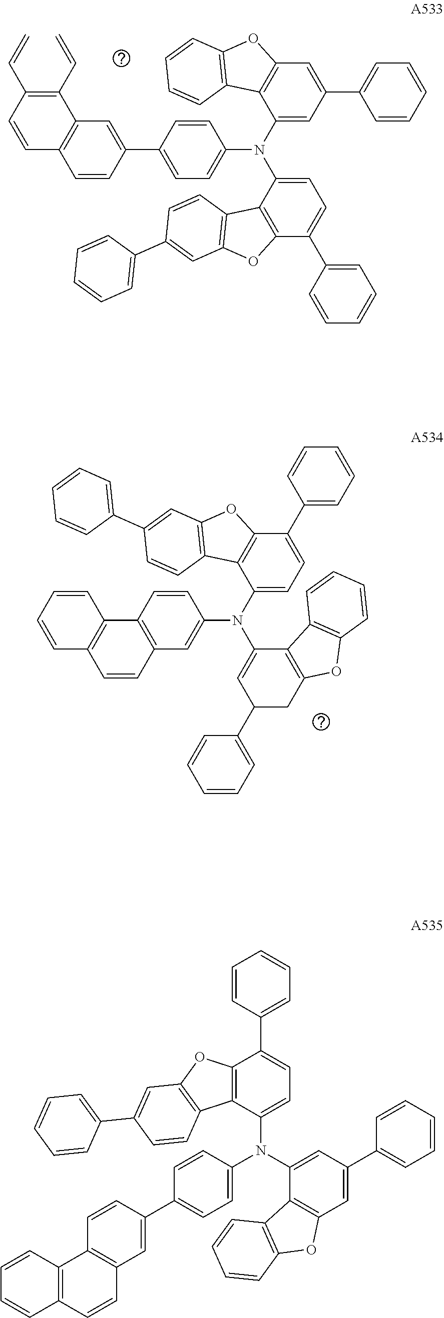 [SMILES: C=Cc1ccc2ccc(-c3ccc(N(c4cc(-c5ccccc5)cc5oc6ccccc6c45)c4ccc(-c5ccccc5)c5oc6cc(-c7ccccc7)ccc6c45)cc3)cc2c1C=C ; C1=C(N(c2ccc3c(ccc4ccccc43)c2)c2ccc(-c3ccccc3)c3oc4cc(-c5ccccc5)ccc4c23)c2c(oc3ccccc23)CC1c1ccccc1 ; c1ccc(-c2ccc3c(c2)oc2c(-c4ccccc4)ccc(N(c4ccc(-c5ccc6c(ccc7ccccc76)c5)cc4)c4cc(-c5ccccc5)cc5oc6ccccc6c45)c23)cc1]

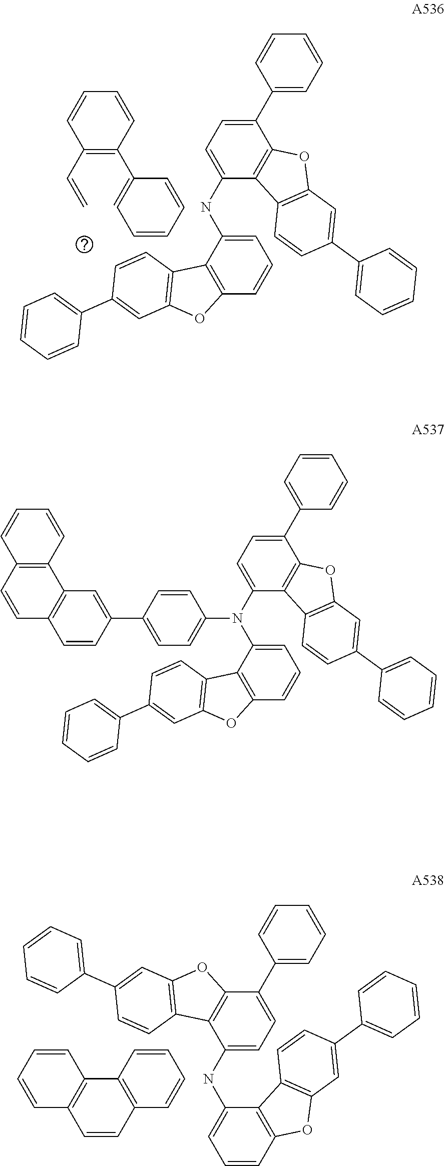

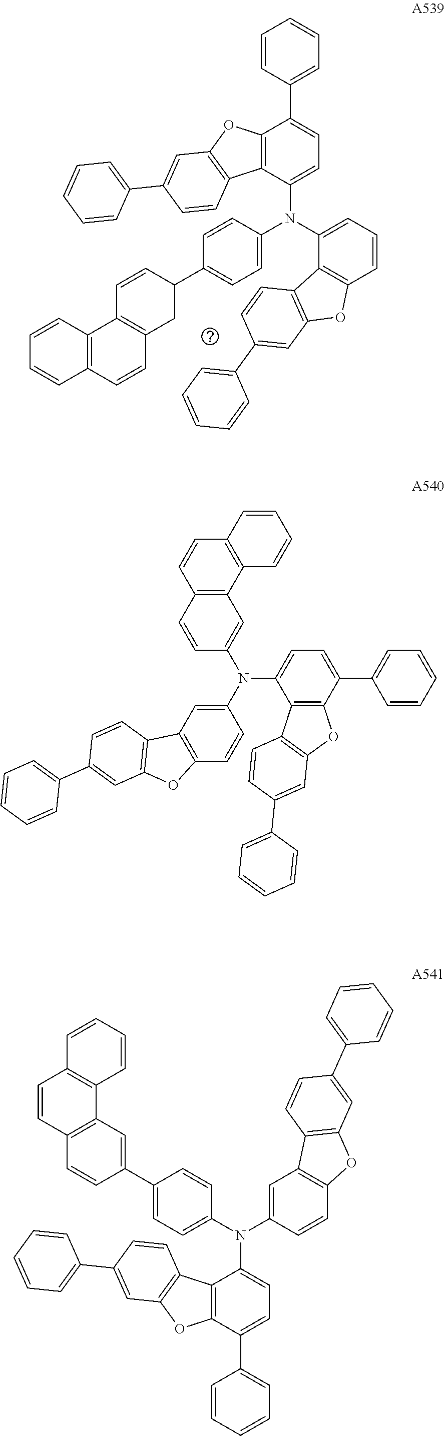 [SMILES: C1=CC(c2ccc(N(c3cccc4oc5cc(-c6ccccc6)ccc5c34)c3ccc(-c4ccccc4)c4oc5cc(-c6ccccc6)ccc5c34)cc2)Cc2ccc3ccccc3c21 ; c1ccc(-c2ccc3c(c2)oc2ccc(N(c4ccc5ccc6ccccc6c5c4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5oc6cc(-c7ccccc7)ccc6c45)cc23)cc1]

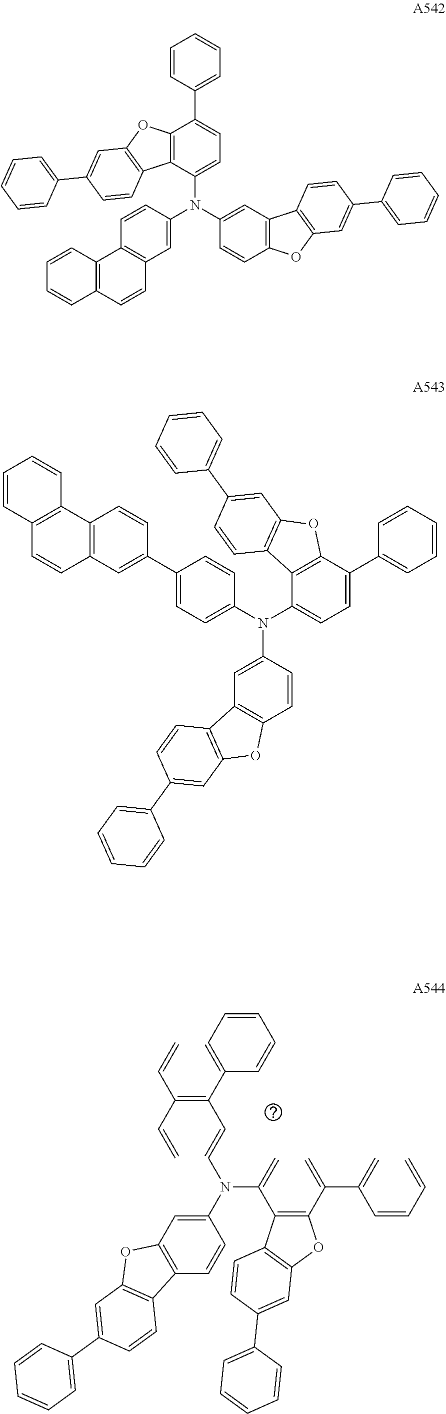 [SMILES: c1ccc(-c2ccc3c(c2)oc2ccc(N(c4ccc5c(ccc6ccccc65)c4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5oc6cc(-c7ccccc7)ccc6c45)cc23)cc1 ; C=C/C=C\C(=C)C(=C)c1oc2cc(-c3ccccc3)ccc2c1C(=C)N(/C=C/C(=C(C=C)C=C)c1ccccc1)c1ccc2c(c1)oc1cc(-c3ccccc3)ccc12]

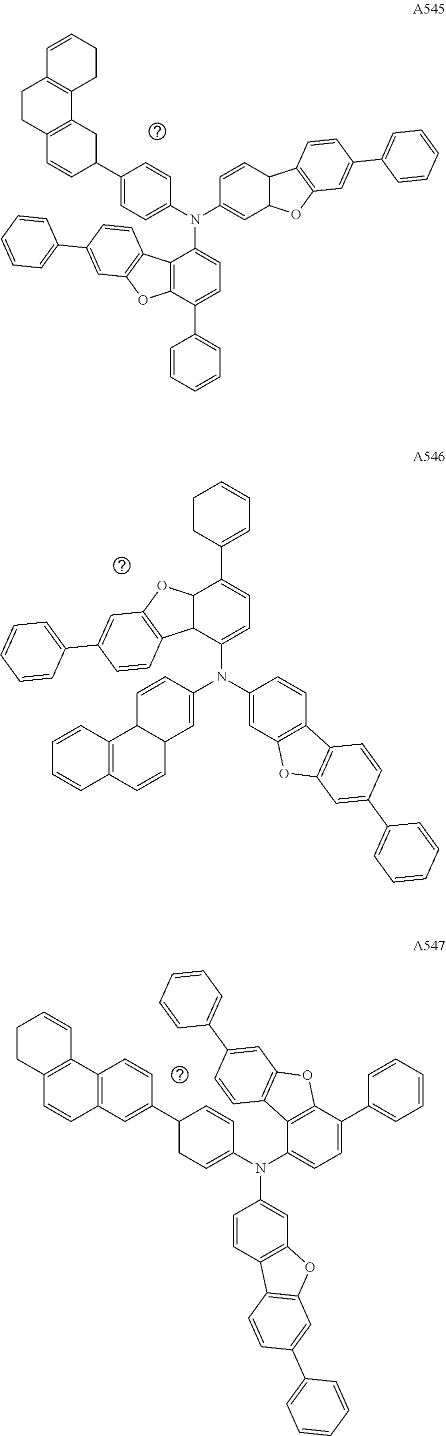 [SMILES: C1=CC2=C(CC1)C1=C(C=CC(c3ccc(N(C4=CC5Oc6cc(-c7ccccc7)ccc6C5C=C4)c4ccc(-c5ccccc5)c5oc6cc(-c7ccccc7)ccc6c45)cc3)C1)CC2 ; C1=CCCC(C2=CC=C(N(C3=CC4C=Cc5ccccc5C4C=C3)c3ccc4c(c3)oc3cc(-c5ccccc5)ccc34)C3c4ccc(-c5ccccc5)cc4OC23)=C1 ; C1=Cc2c(ccc3cc(C4C=CC(N(c5ccc6c(c5)oc5cc(-c7ccccc7)ccc56)c5ccc(-c6ccccc6)c6oc7cc(-c8ccccc8)ccc7c56)=CC4)ccc23)CC1]

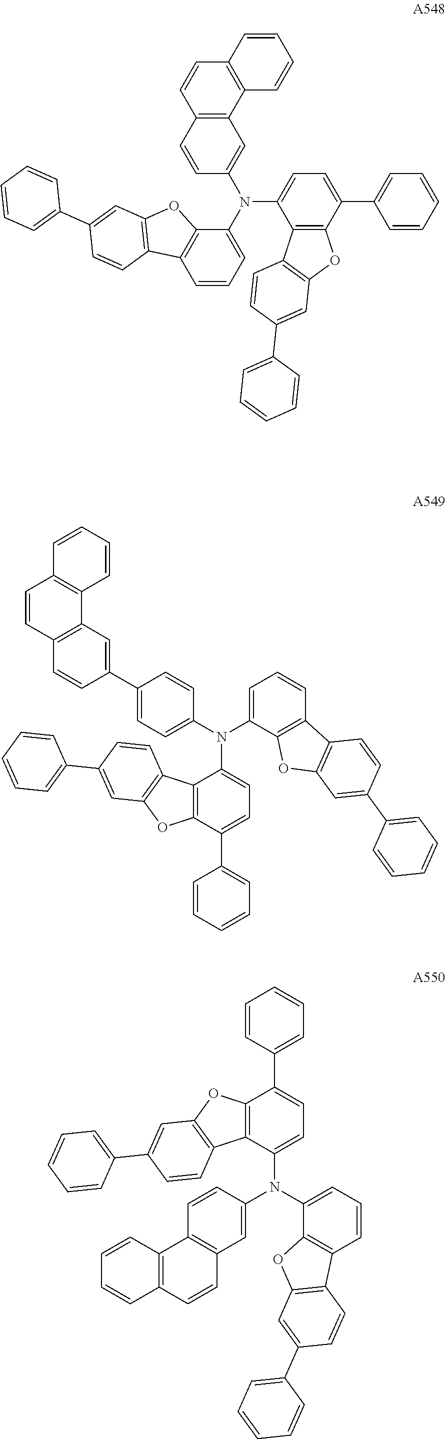 [SMILES: c1ccc(-c2ccc3c(c2)oc2c(N(c4ccc5ccc6ccccc6c5c4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5oc6cc(-c7ccccc7)ccc6c45)cccc23)cc1 ; c1ccc(-c2ccc3c(c2)oc2c(N(c4ccc5c(ccc6ccccc65)c4)c4ccc(-c5ccccc5)c5oc6cc(-c7ccccc7)ccc6c45)cccc23)cc1]

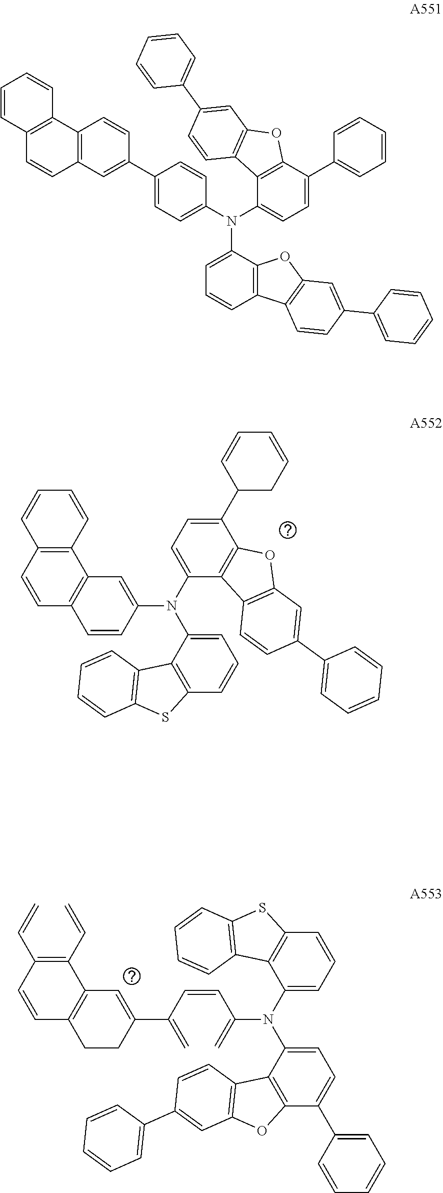 [SMILES: c1ccc(-c2ccc3c(c2)oc2c(N(c4ccc(-c5ccc6c(ccc7ccccc76)c5)cc4)c4ccc(-c5ccccc5)c5oc6cc(-c7ccccc7)ccc6c45)cccc23)cc1 ; C1=CCC(c2ccc(N(c3ccc4ccc5ccccc5c4c3)c3cccc4sc5ccccc5c34)c3c2oc2cc(-c4ccccc4)ccc23)C=C1 ; C=Cc1ccc2c(c1C=C)C=C(C(=C)/C=C\C(=C)N(c1cccc3sc4ccccc4c13)c1ccc(-c3ccccc3)c3oc4cc(-c5ccccc5)ccc4c13)CC2]

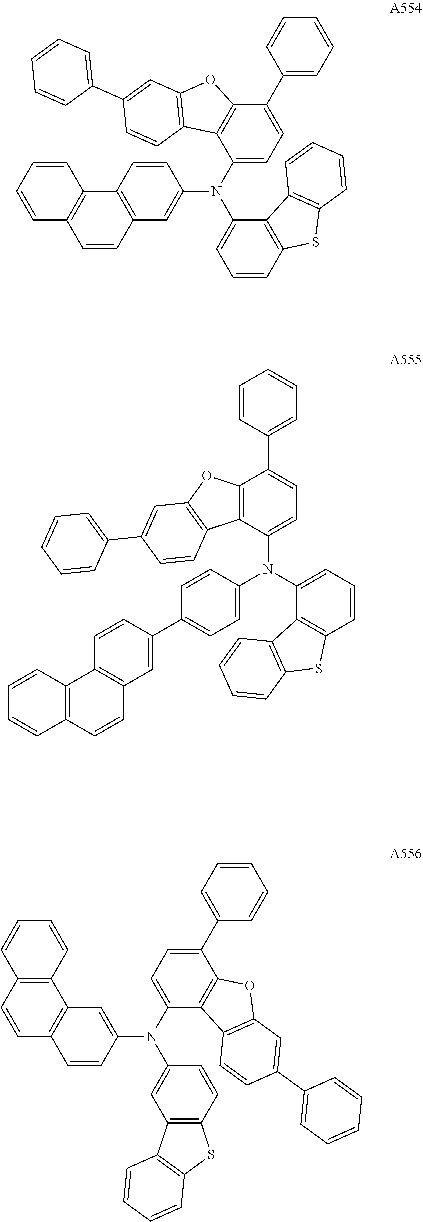 [SMILES: c1ccc(-c2ccc3c(c2)oc2c(-c4ccccc4)ccc(N(c4ccc5c(ccc6ccccc65)c4)c4cccc5sc6ccccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5sc6ccccc6c45)c23)cc1 ; c1ccc(-c2ccc3c(c2)oc2c(-c4ccccc4)ccc(N(c4ccc5ccc6ccccc6c5c4)c4ccc5sc6ccccc6c5c4)c23)cc1]

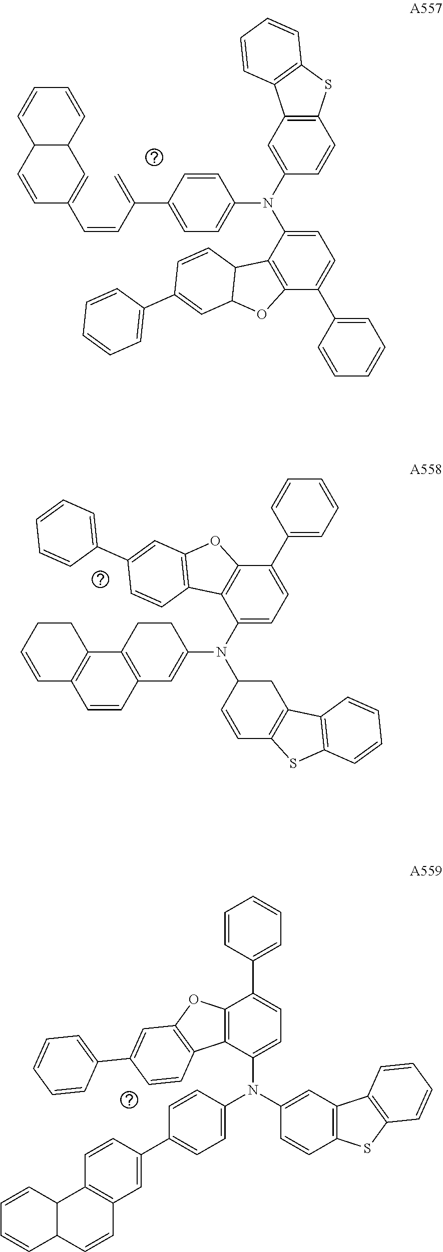 [SMILES: C=C(/C=C\C1=CC2C=CC=CC2C=C1)c1ccc(N(c2ccc3sc4ccccc4c3c2)c2ccc(-c3ccccc3)c3c2C2C=CC(c4ccccc4)=CC2O3)cc1 ; C1=Cc2ccc3c(c2CC1)CCC(N(c1ccc(-c2ccccc2)c2oc4cc(-c5ccccc5)ccc4c12)C1C=Cc2sc4ccccc4c2C1)=C3 ; C1=CC2C=Cc3cc(-c4ccc(N(c5ccc6sc7ccccc7c6c5)c5ccc(-c6ccccc6)c6oc7cc(-c8ccccc8)ccc7c56)cc4)ccc3C2C=C1]

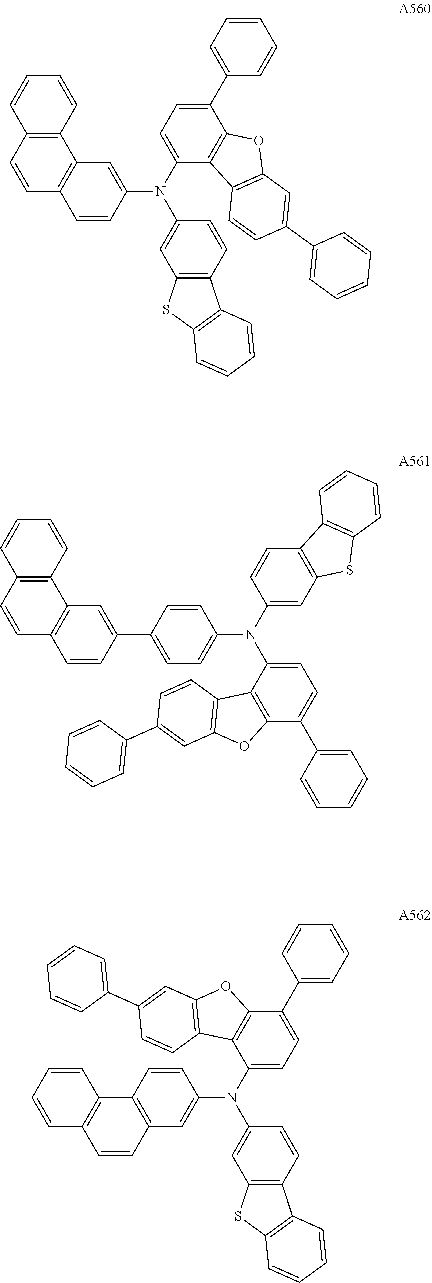 [SMILES: c1ccc(-c2ccc3c(c2)oc2c(-c4ccccc4)ccc(N(c4ccc5c(c4)sc4ccccc45)c4ccc5ccc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5c(c4)sc4ccccc45)c23)cc1 ; c1ccc(-c2ccc3c(c2)oc2c(-c4ccccc4)ccc(N(c4ccc5c(ccc6ccccc65)c4)c4ccc5c(c4)sc4ccccc45)c23)cc1]

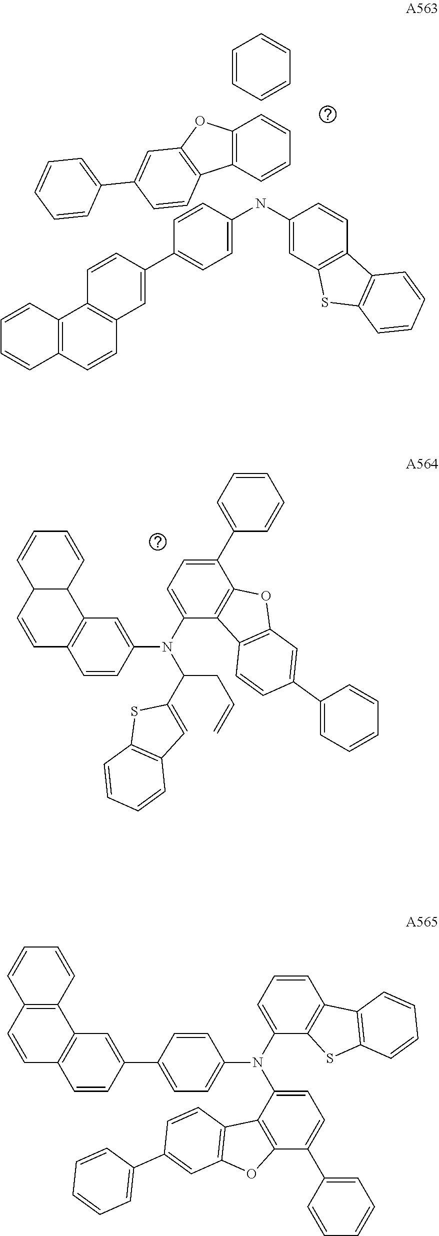

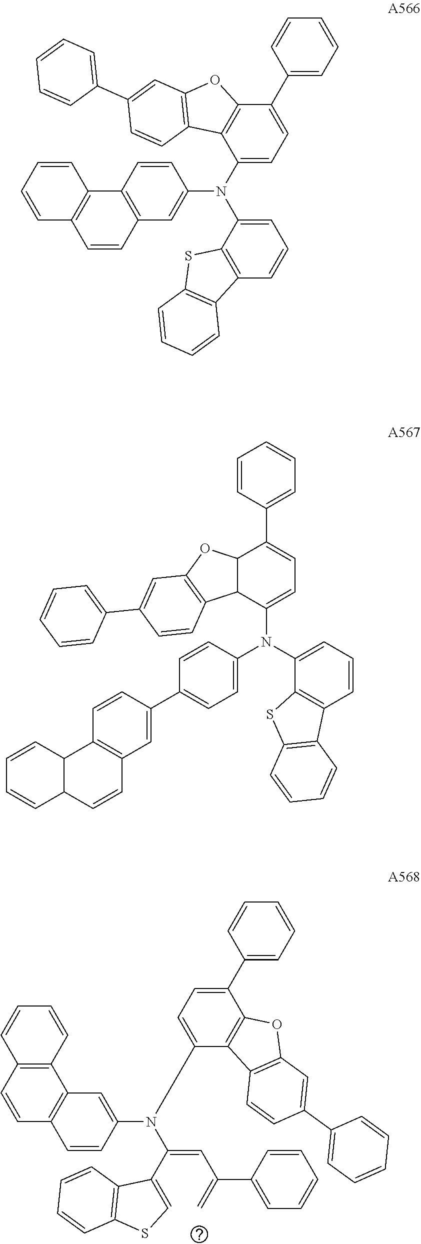 [SMILES: c1ccc(-c2ccc3c(c2)oc2c(-c4ccccc4)ccc(N(c4ccc5c(ccc6ccccc65)c4)c4cccc5c4sc4ccccc45)c23)cc1 ; C1=CC2C=Cc3cc(-c4ccc(N(C5=CC=C(c6ccccc6)C6Oc7cc(-c8ccccc8)ccc7C56)c5cccc6c5sc5ccccc56)cc4)ccc3C2C=C1 ; C=C(/C=C(\c1csc2ccccc12)N(c1ccc2ccc3ccccc3c2c1)c1ccc(-c2ccccc2)c2oc3cc(-c4ccccc4)ccc3c12)c1ccccc1]

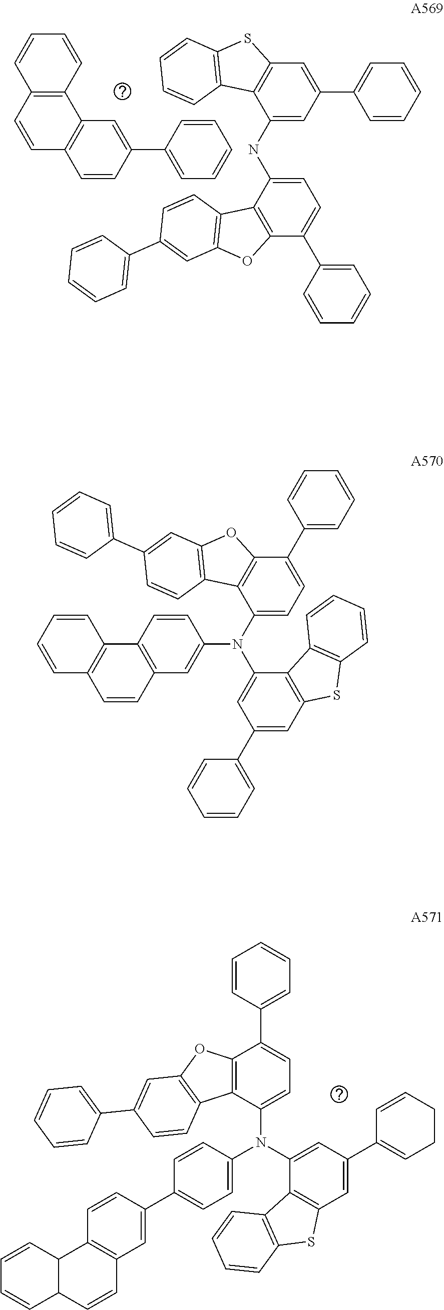

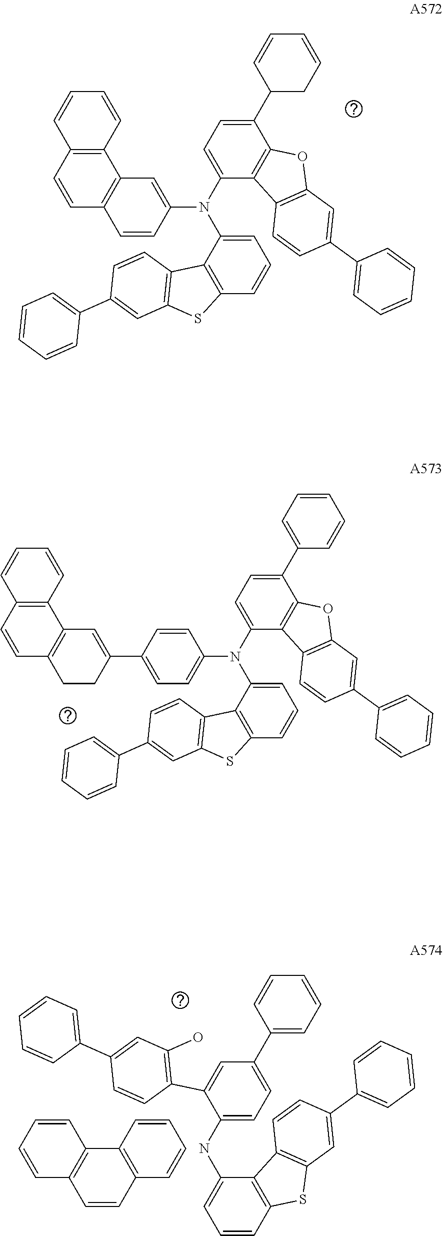

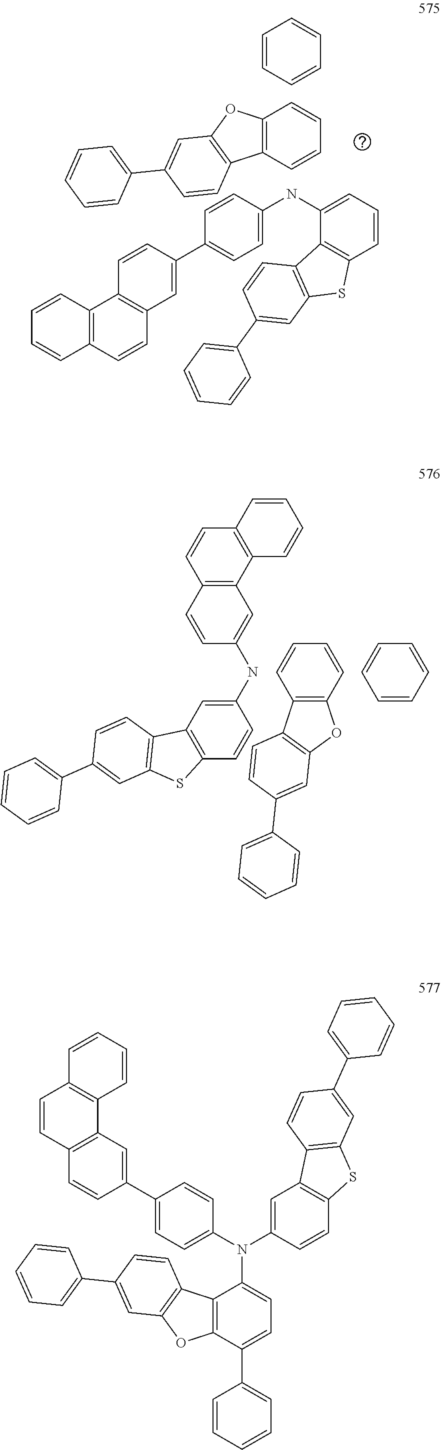

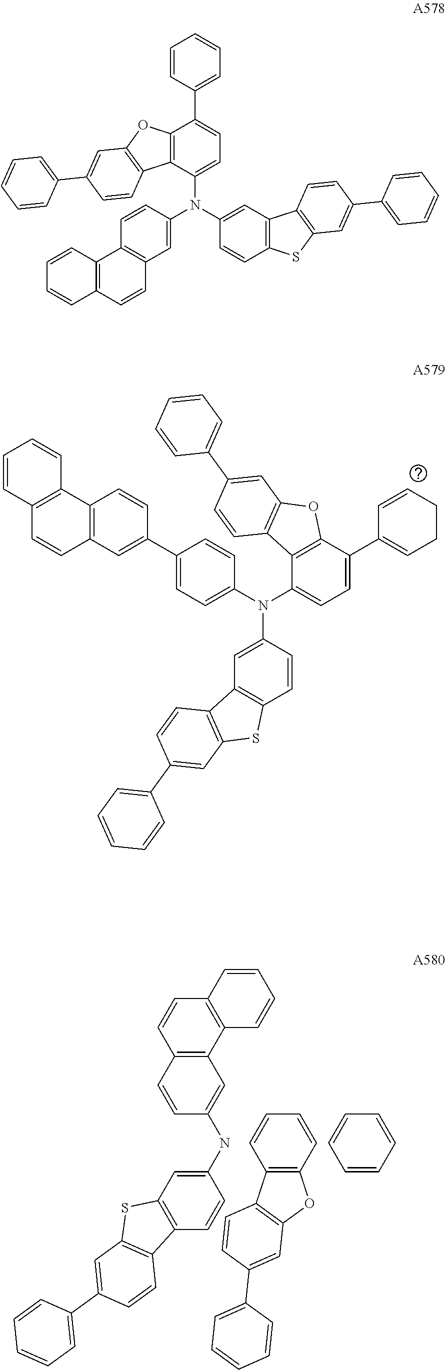

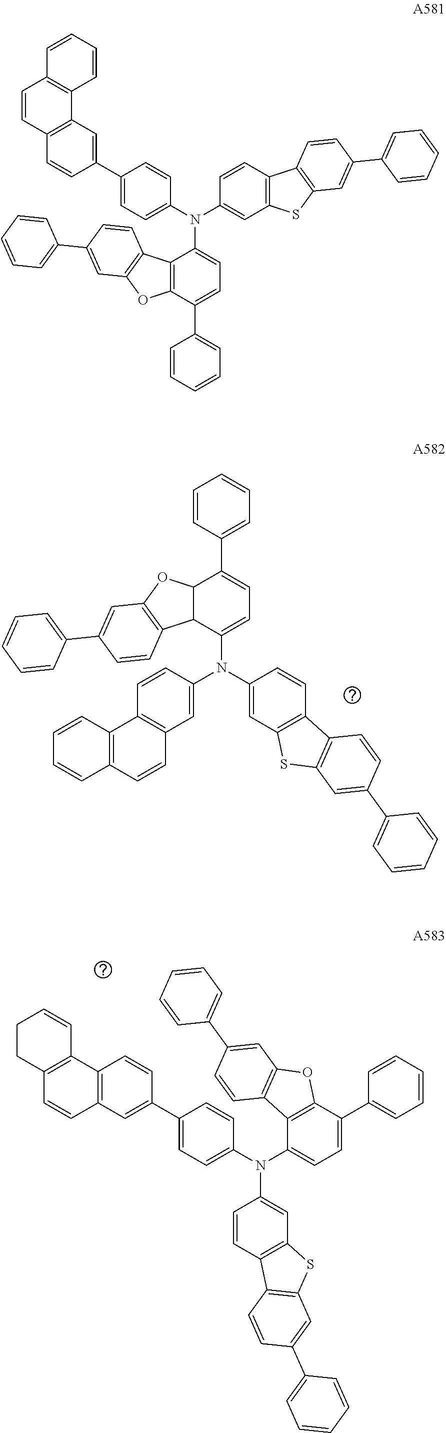 [SMILES: c1ccc(-c2ccc3c(c2)oc2c(-c4ccccc4)ccc(N(c4ccc(-c5ccc6ccc7ccccc7c6c5)cc4)c4ccc5c(c4)sc4cc(-c6ccccc6)ccc45)c23)cc1 ; C1=C(c2ccccc2)C2Oc3cc(-c4ccccc4)ccc3C2C(N(c2ccc3c(ccc4ccccc43)c2)c2ccc3c(c2)sc2cc(-c4ccccc4)ccc23)=C1 ; C1=Cc2c(ccc3cc(-c4ccc(N(c5ccc6c(c5)sc5cc(-c7ccccc7)ccc56)c5ccc(-c6ccccc6)c6oc7cc(-c8ccccc8)ccc7c56)cc4)ccc23)CC1]

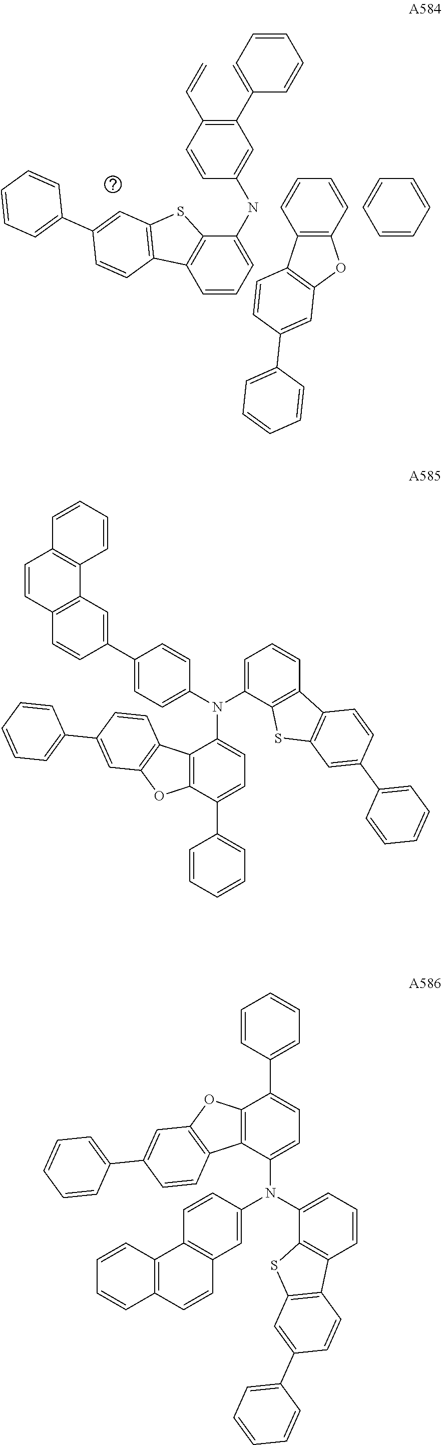

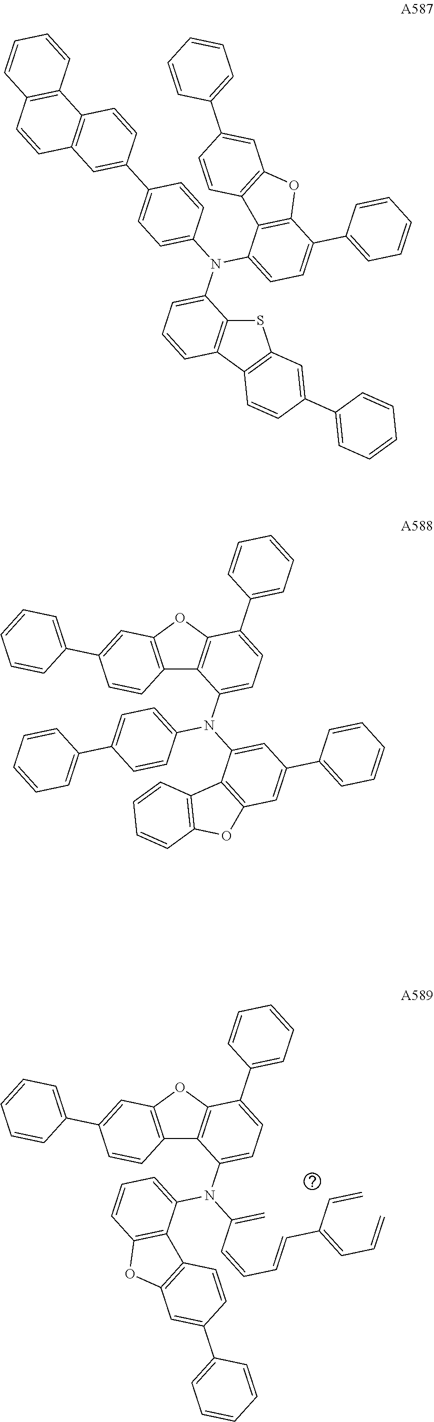 [SMILES: c1ccc(-c2ccc3c(c2)oc2c(-c4ccccc4)ccc(N(c4ccc(-c5ccc6c(ccc7ccccc76)c5)cc4)c4cccc5c4sc4cc(-c6ccccc6)ccc45)c23)cc1 ; c1ccc(-c2ccc(N(c3cc(-c4ccccc4)cc4oc5ccccc5c34)c3ccc(-c4ccccc4)c4oc5cc(-c6ccccc6)ccc5c34)cc2)cc1 ; C=C/C=C(C=C)/C=C/C=C\C(=C)N(c1cccc2oc3cc(-c4ccccc4)ccc3c12)c1ccc(-c2ccccc2)c2oc3cc(-c4ccccc4)ccc3c12]

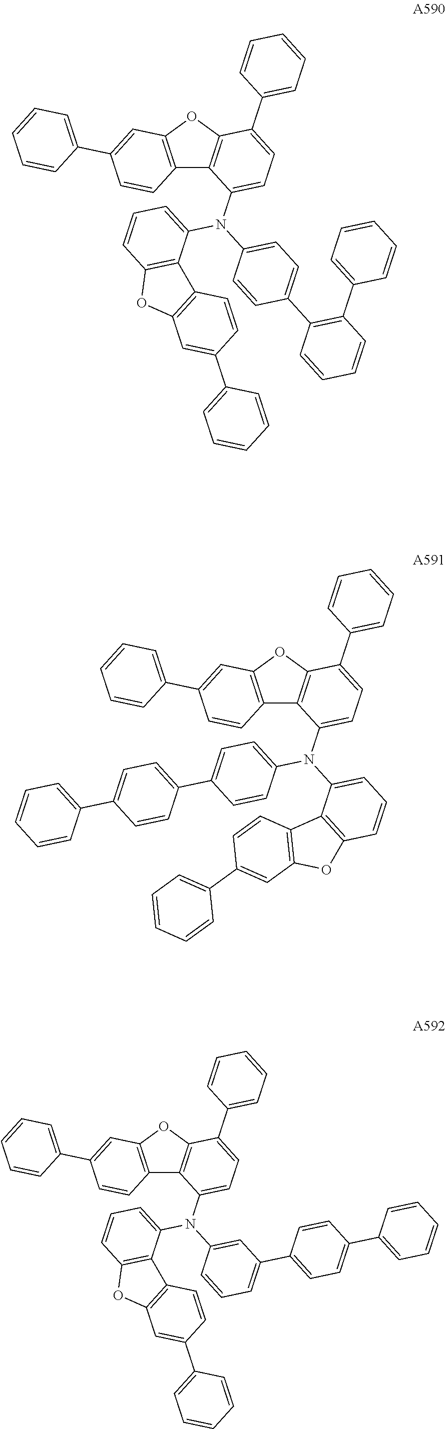

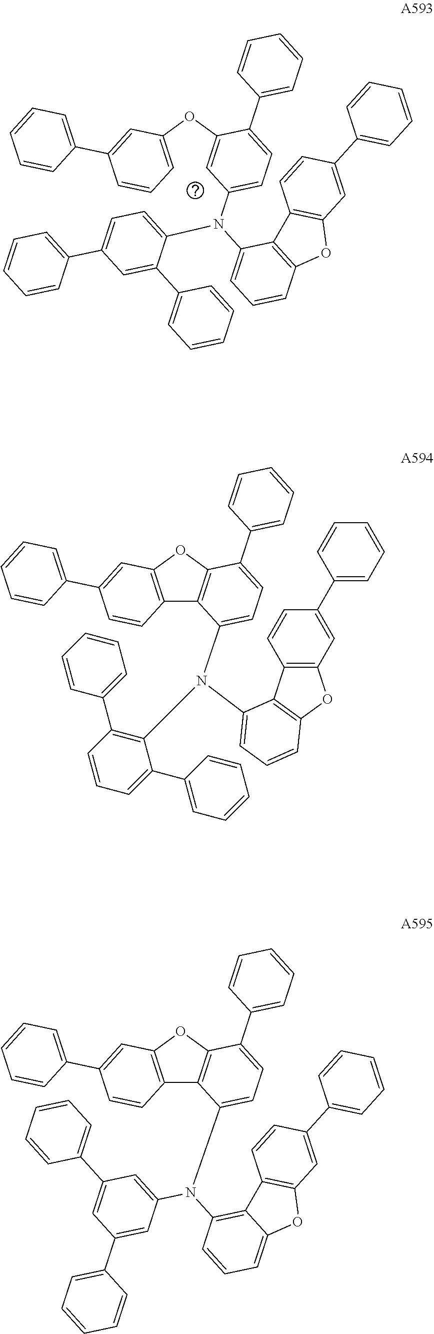 [SMILES: c1ccc(-c2cccc(Oc3cc(N(c4ccc(-c5ccccc5)cc4-c4ccccc4)c4cccc5oc6cc(-c7ccccc7)ccc6c45)ccc3-c3ccccc3)c2)cc1 ; c1ccc(-c2ccc3c(c2)oc2cccc(N(c4c(-c5ccccc5)cccc4-c4ccccc4)c4ccc(-c5ccccc5)c5oc6cc(-c7ccccc7)ccc6c45)c23)cc1 ; c1ccc(-c2cc(-c3ccccc3)cc(N(c3cccc4oc5cc(-c6ccccc6)ccc5c34)c3ccc(-c4ccccc4)c4oc5cc(-c6ccccc6)ccc5c34)c2)cc1]

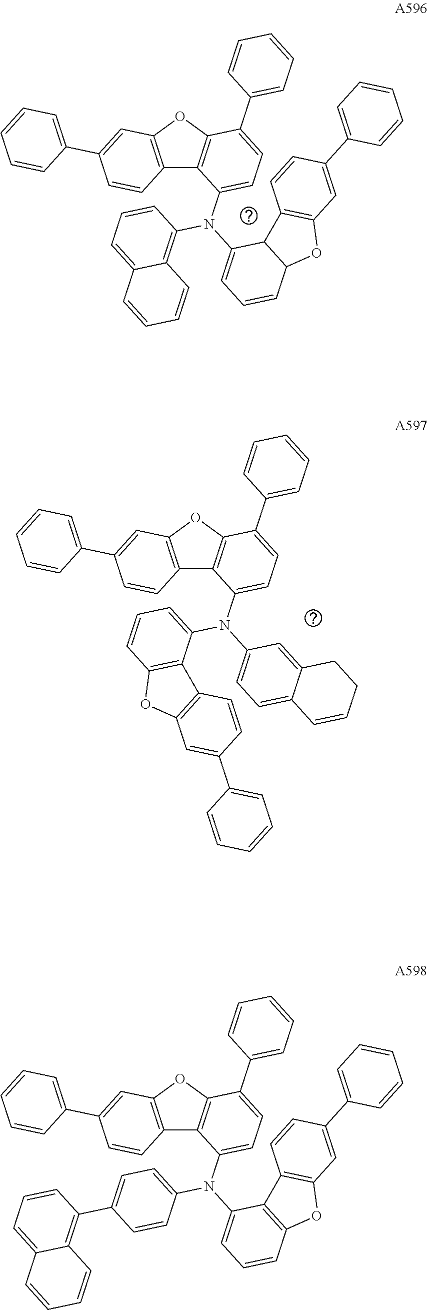 [SMILES: C1=CC2Oc3cc(-c4ccccc4)ccc3C2C(N(c2cccc3ccccc23)c2ccc(-c3ccccc3)c3oc4cc(-c5ccccc5)ccc4c23)=C1 ; C1=Cc2ccc(N(c3cccc4oc5cc(-c6ccccc6)ccc5c34)c3ccc(-c4ccccc4)c4oc5cc(-c6ccccc6)ccc5c34)cc2CC1 ; c1ccc(-c2ccc3c(c2)oc2cccc(N(c4ccc(-c5cccc6ccccc56)cc4)c4ccc(-c5ccccc5)c5oc6cc(-c7ccccc7)ccc6c45)c23)cc1]

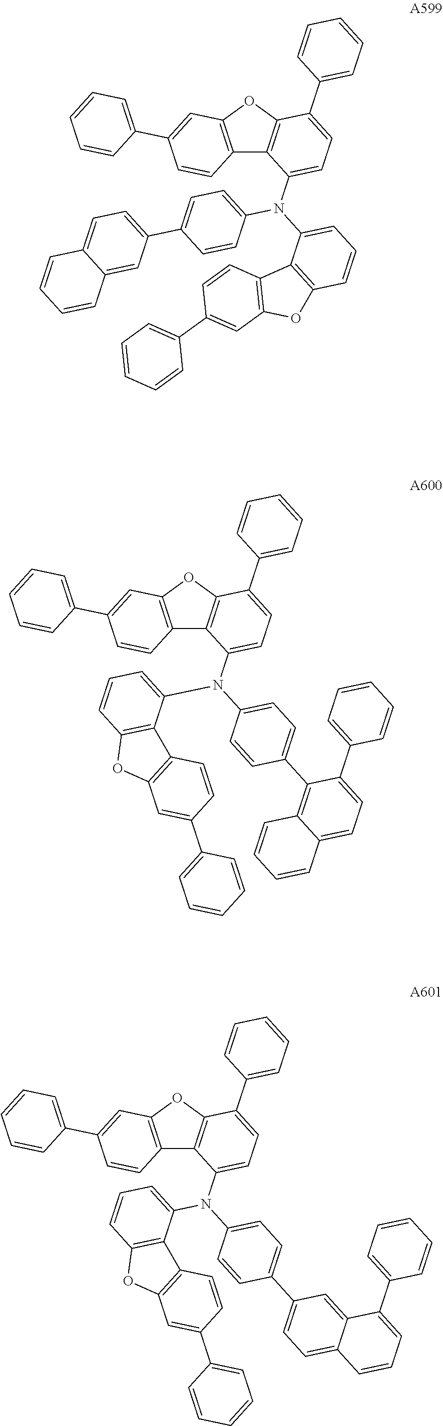 [SMILES: c1ccc(-c2ccc3c(c2)oc2cccc(N(c4ccc(-c5ccc6ccccc6c5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5c(-c6ccccc6)ccc6ccccc56)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6cccc(-c7ccccc7)c6c5)cc4)c4ccc(-c5ccccc5)c5oc6cc(-c7ccccc7)ccc6c45)c23)cc1]

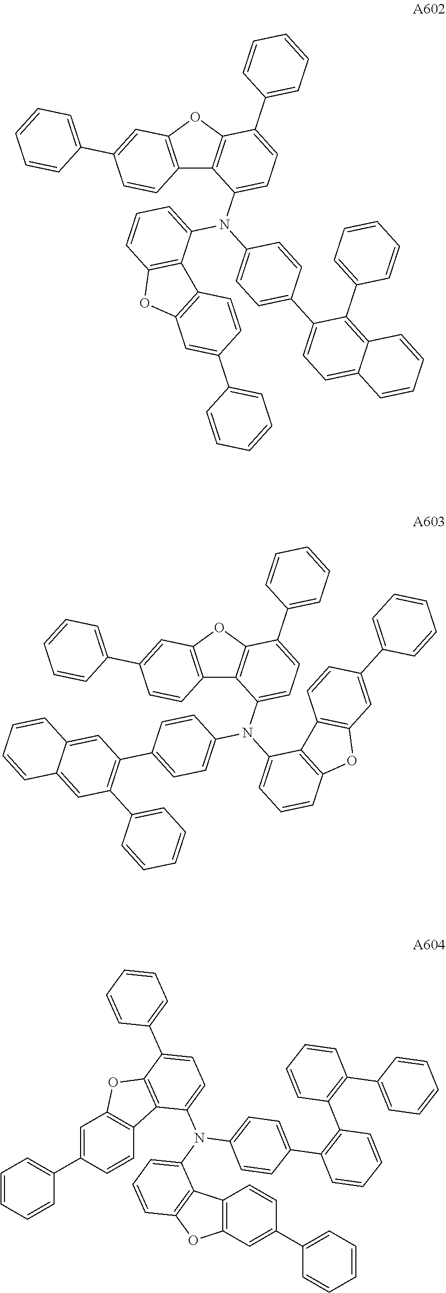 [SMILES: c1ccc(-c2ccc3c(c2)oc2cccc(N(c4ccc(-c5ccc6ccccc6c5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5cc6ccccc6cc5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccccc5-c5ccccc5-c5ccccc5)cc4)c4ccc(-c5ccccc5)c5oc6cc(-c7ccccc7)ccc6c45)c23)cc1]

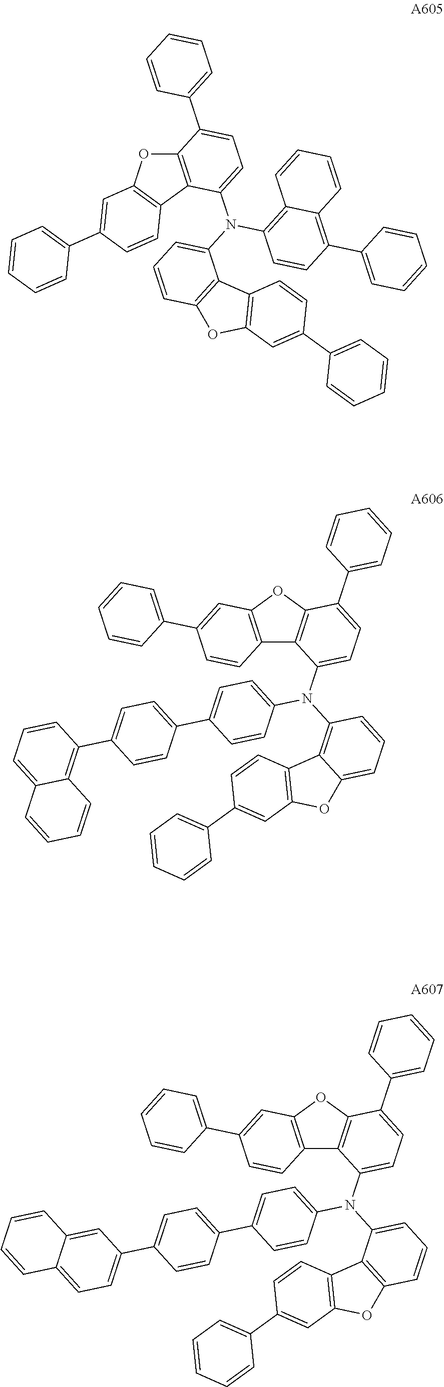 [SMILES: c1ccc(-c2ccc3c(c2)oc2cccc(N(c4ccc(-c5ccccc5)c5ccccc45)c4ccc(-c5ccccc5)c5oc6cc(-c7ccccc7)ccc6c45)c23)cc1 ; c1ccc(-c2ccc3c(c2)oc2cccc(N(c4ccc(-c5ccc(-c6cccc7ccccc67)cc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5oc6cc(-c7ccccc7)ccc6c45)c23)cc1]

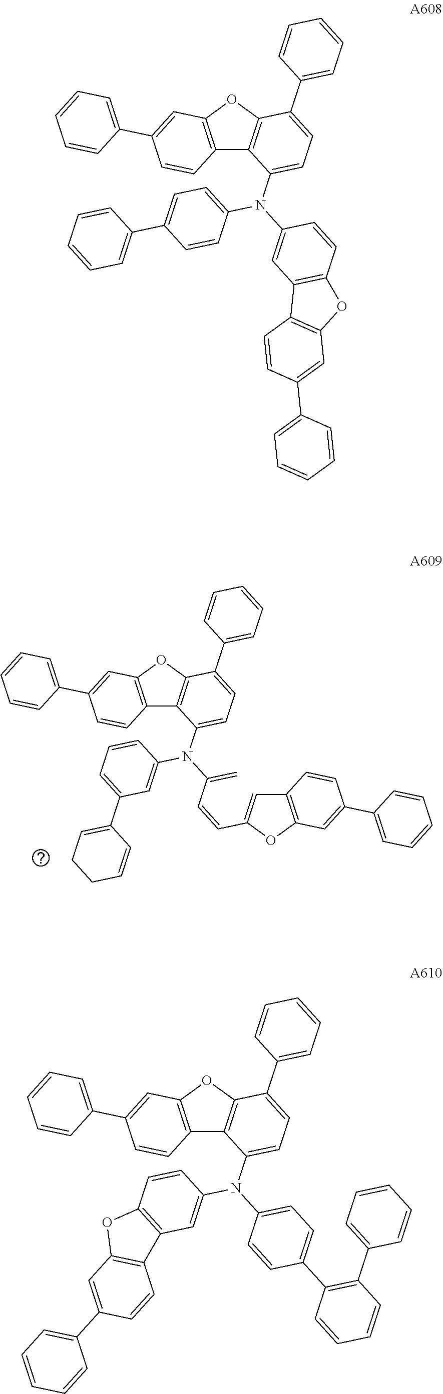 [SMILES: c1ccc(-c2ccc(N(c3ccc4oc5cc(-c6ccccc6)ccc5c4c3)c3ccc(-c4ccccc4)c4oc5cc(-c6ccccc6)ccc5c34)cc2)cc1 ; C=C(/C=C\c1cc2ccc(-c3ccccc3)cc2o1)N(c1cccc(C2=CCCC=C2)c1)c1ccc(-c2ccccc2)c2oc3cc(-c4ccccc4)ccc3c12 ; c1ccc(-c2ccc3c(c2)oc2ccc(N(c4ccc(-c5ccccc5-c5ccccc5)cc4)c4ccc(-c5ccccc5)c5oc6cc(-c7ccccc7)ccc6c45)cc23)cc1]

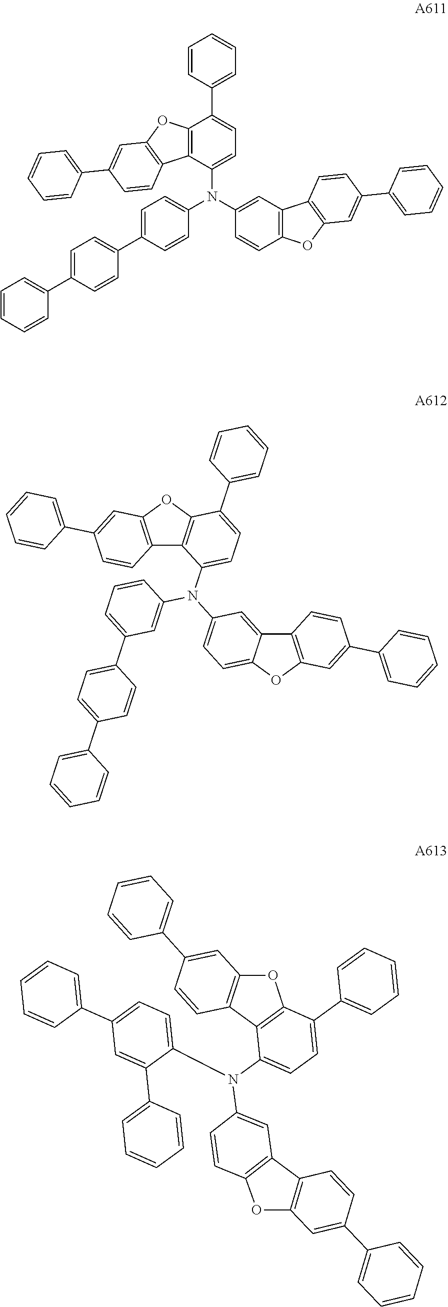 [SMILES: c1ccc(-c2ccc(-c3ccc(N(c4ccc5oc6cc(-c7ccccc7)ccc6c5c4)c4ccc(-c5ccccc5)c5oc6cc(-c7ccccc7)ccc6c45)cc3)cc2)cc1 ; c1ccc(-c2ccc(-c3cccc(N(c4ccc5oc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4oc5cc(-c6ccccc6)ccc5c4c3)c3ccc(-c4ccccc4)c4oc5cc(-c6ccccc6)ccc5c34)c(-c3ccccc3)c2)cc1]

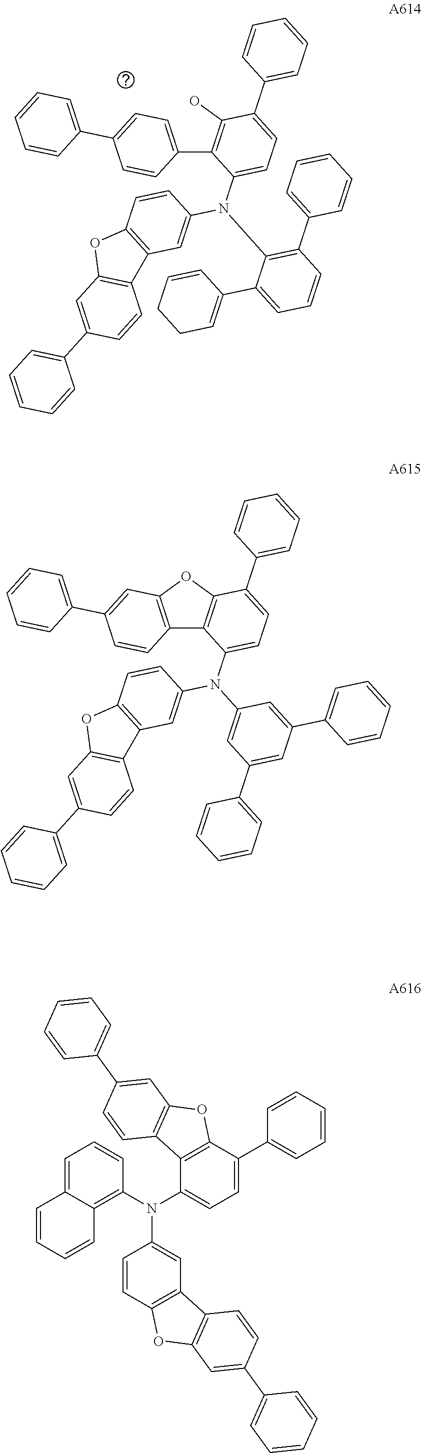 [SMILES: Oc1c(-c2ccccc2)ccc(N(c2ccc3oc4cc(-c5ccccc5)ccc4c3c2)c2c(C3=CCCC=C3)cccc2-c2ccccc2)c1-c1ccc(-c2ccccc2)cc1 ; c1ccc(-c2cc(-c3ccccc3)cc(N(c3ccc4oc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2ccc(N(c4cccc5ccccc45)c4ccc(-c5ccccc5)c5oc6cc(-c7ccccc7)ccc6c45)cc23)cc1]

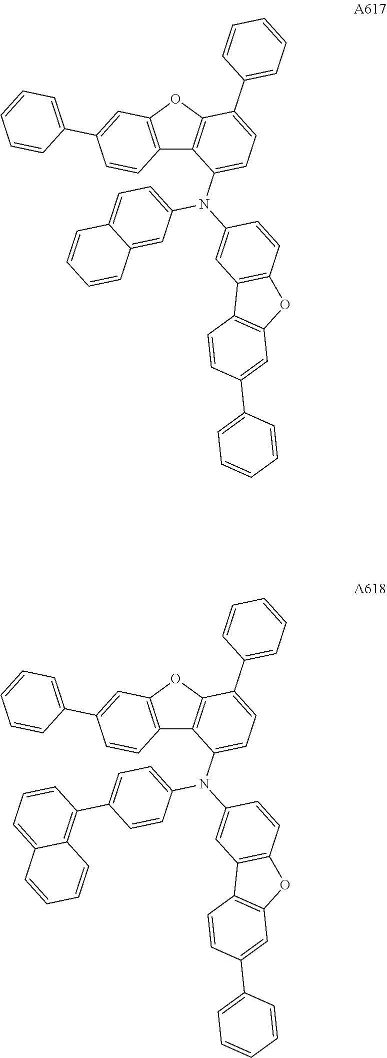 [SMILES: c1ccc(-c2ccc3c(c2)oc2ccc(N(c4ccc5ccccc5c4)c4ccc(-c5ccccc5)c5oc6cc(-c7ccccc7)ccc6c45)cc23)cc1 ; c1ccc(-c2ccc3c(c2)oc2ccc(N(c4ccc(-c5cccc6ccccc56)cc4)c4ccc(-c5ccccc5)c5oc6cc(-c7ccccc7)ccc6c45)cc23)cc1]

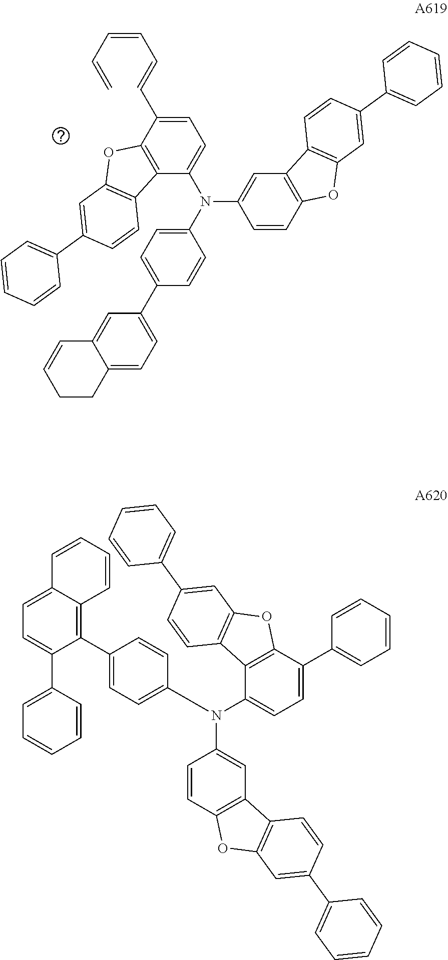 [SMILES: C=C/C=C\C=C\c1ccc(N(c2ccc(-c3ccc4c(c3)C=CCC4)cc2)c2ccc3oc4cc(-c5ccccc5)ccc4c3c2)c2c1oc1cc(-c3ccccc3)ccc12 ; c1ccc(-c2ccc3c(c2)oc2ccc(N(c4ccc(-c5c(-c6ccccc6)ccc6ccccc56)cc4)c4ccc(-c5ccccc5)c5oc6cc(-c7ccccc7)ccc6c45)cc23)cc1]

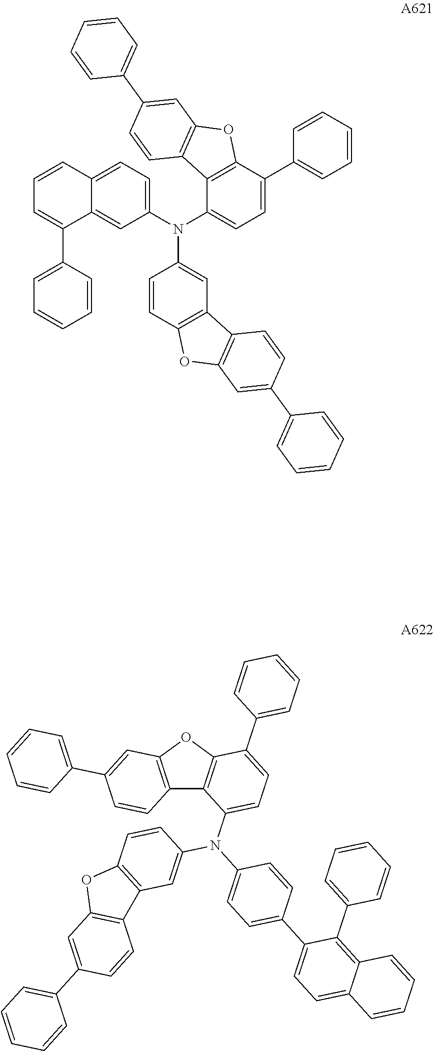 [SMILES: c1ccc(-c2ccc3c(c2)oc2ccc(N(c4ccc5cccc(-c6ccccc6)c5c4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6ccccc6c5-c5ccccc5)cc4)c4ccc(-c5ccccc5)c5oc6cc(-c7ccccc7)ccc6c45)cc23)cc1]

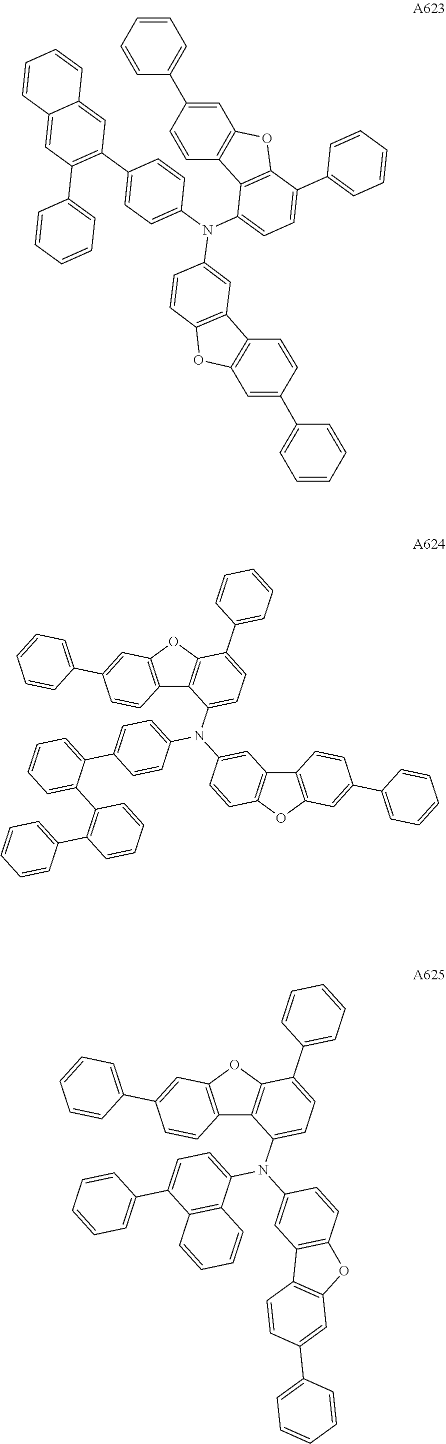 [SMILES: c1ccc(-c2ccc3c(c2)oc2ccc(N(c4ccc(-c5cc6ccccc6cc5-c5ccccc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccccc5-c5ccccc5-c5ccccc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccccc5)c5ccccc45)c4ccc(-c5ccccc5)c5oc6cc(-c7ccccc7)ccc6c45)cc23)cc1]

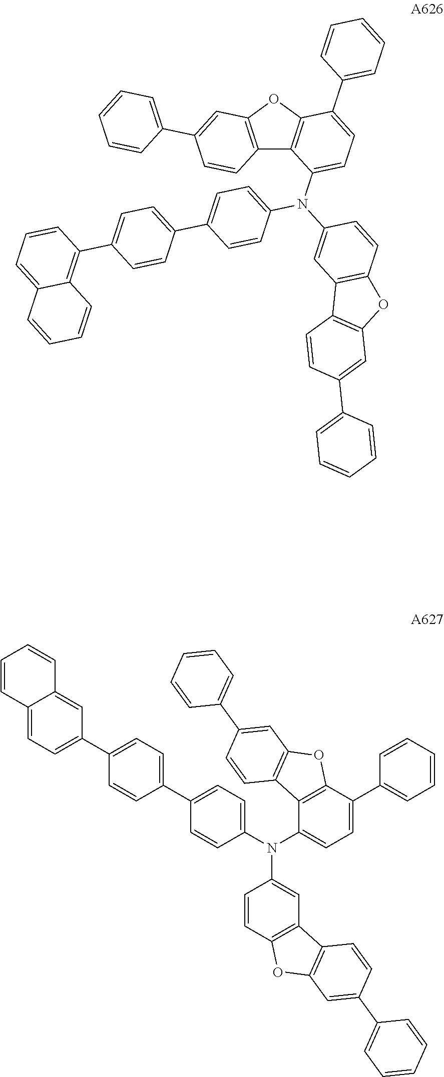 [SMILES: c1ccc(-c2ccc3c(c2)oc2ccc(N(c4ccc(-c5ccc(-c6cccc7ccccc67)cc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc(-c6ccc7ccccc7c6)cc5)cc4)c4ccc(-c5ccccc5)c5oc6cc(-c7ccccc7)ccc6c45)cc23)cc1]

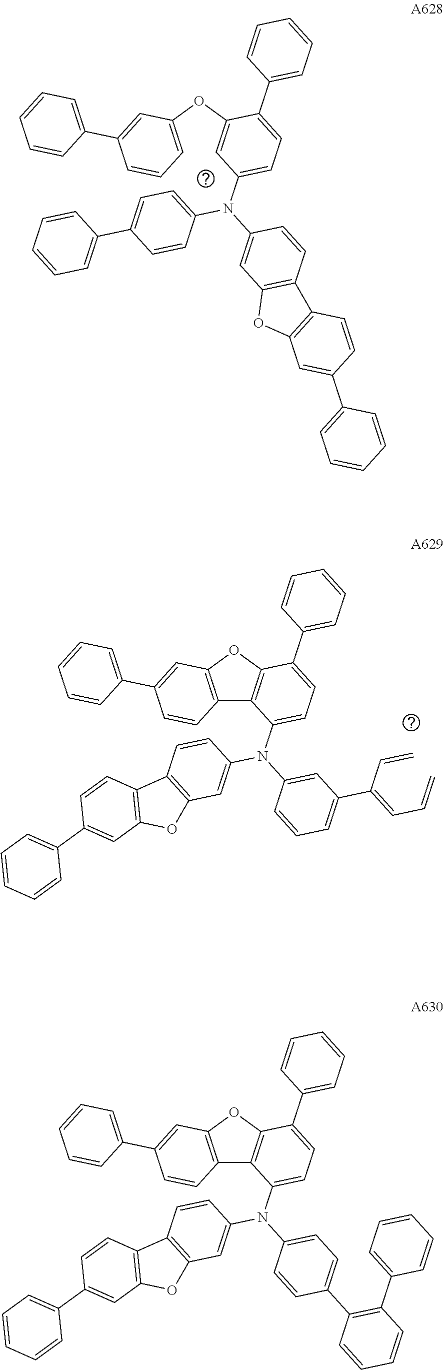 [SMILES: c1ccc(-c2ccc(N(c3ccc(-c4ccccc4)c(Oc4cccc(-c5ccccc5)c4)c3)c3ccc4c(c3)oc3cc(-c5ccccc5)ccc34)cc2)cc1 ; C=C/C=C(\C=C)c1cccc(N(c2ccc3c(c2)oc2cc(-c4ccccc4)ccc23)c2ccc(-c3ccccc3)c3oc4cc(-c5ccccc5)ccc4c23)c1 ; c1ccc(-c2ccc3c(c2)oc2cc(N(c4ccc(-c5ccccc5-c5ccccc5)cc4)c4ccc(-c5ccccc5)c5oc6cc(-c7ccccc7)ccc6c45)ccc23)cc1]

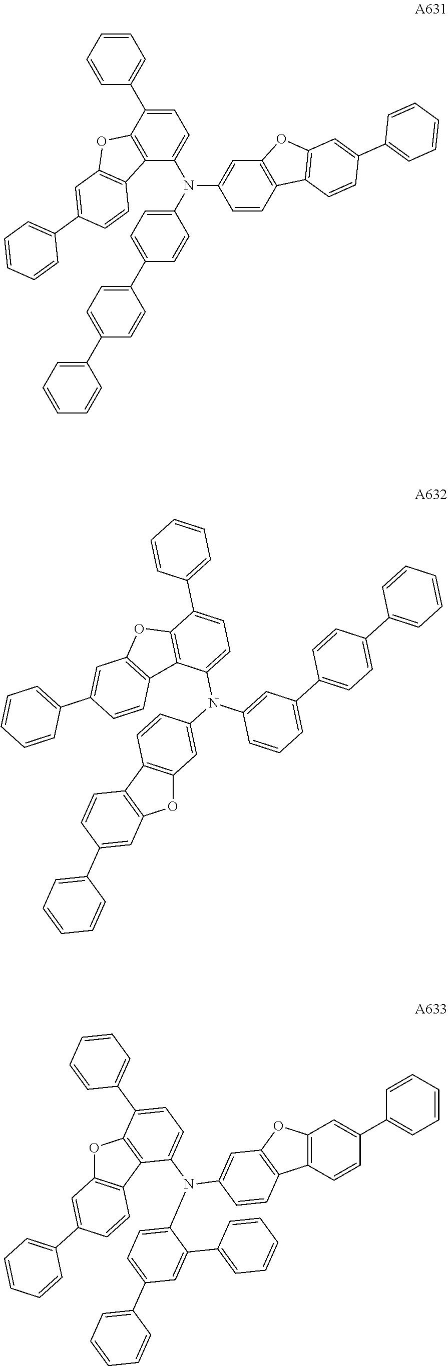 [SMILES: c1ccc(-c2ccc(-c3ccc(N(c4ccc5c(c4)oc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)oc4cc(-c6ccccc6)ccc45)c4ccc(-c5ccccc5)c5oc6cc(-c7ccccc7)ccc6c45)c3)cc2)cc1 ; c1ccc(-c2ccc(N(c3ccc4c(c3)oc3cc(-c5ccccc5)ccc34)c3ccc(-c4ccccc4)c4oc5cc(-c6ccccc6)ccc5c34)c(-c3ccccc3)c2)cc1]

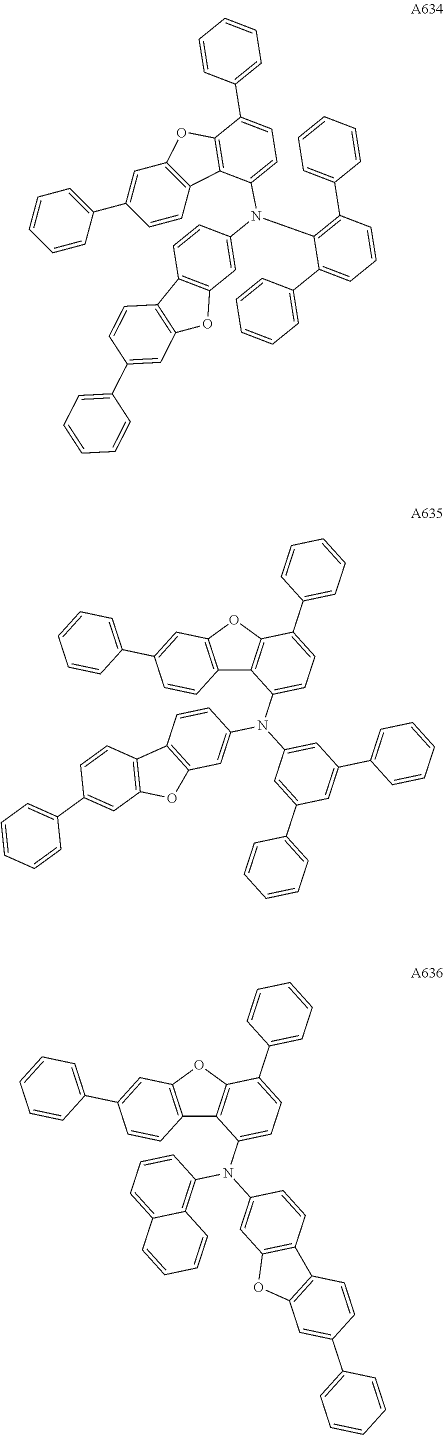 [SMILES: c1ccc(-c2ccc3c(c2)oc2cc(N(c4c(-c5ccccc5)cccc4-c4ccccc4)c4ccc(-c5ccccc5)c5oc6cc(-c7ccccc7)ccc6c45)ccc23)cc1 ; c1ccc(-c2cc(-c3ccccc3)cc(N(c3ccc4c(c3)oc3cc(-c5ccccc5)ccc34)c3ccc(-c4ccccc4)c4oc5cc(-c6ccccc6)ccc5c34)c2)cc1 ; c1ccc(-c2ccc3c(c2)oc2cc(N(c4cccc5ccccc45)c4ccc(-c5ccccc5)c5oc6cc(-c7ccccc7)ccc6c45)ccc23)cc1]

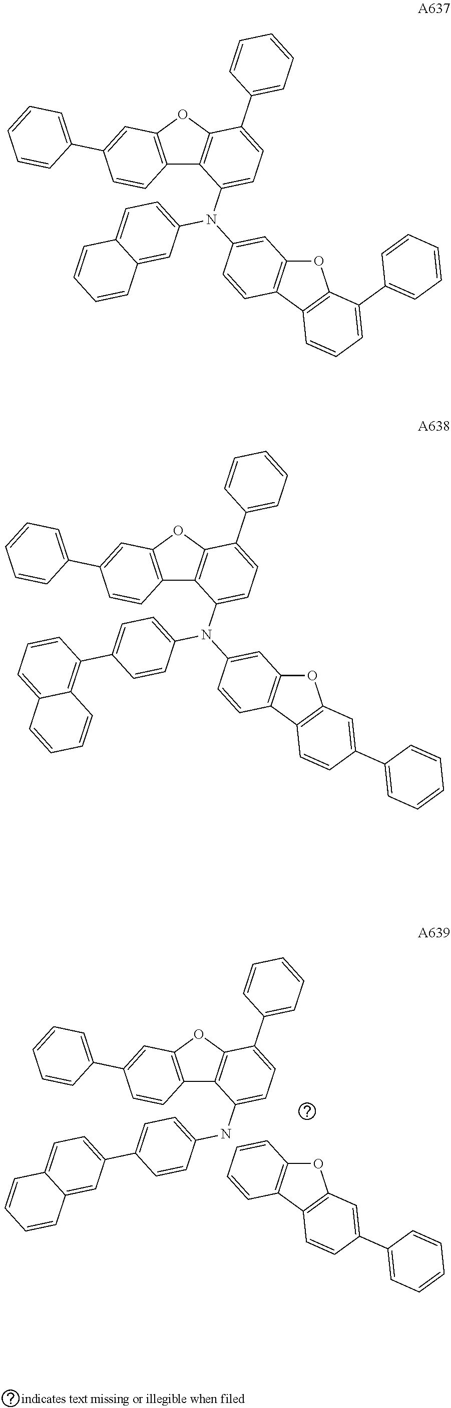

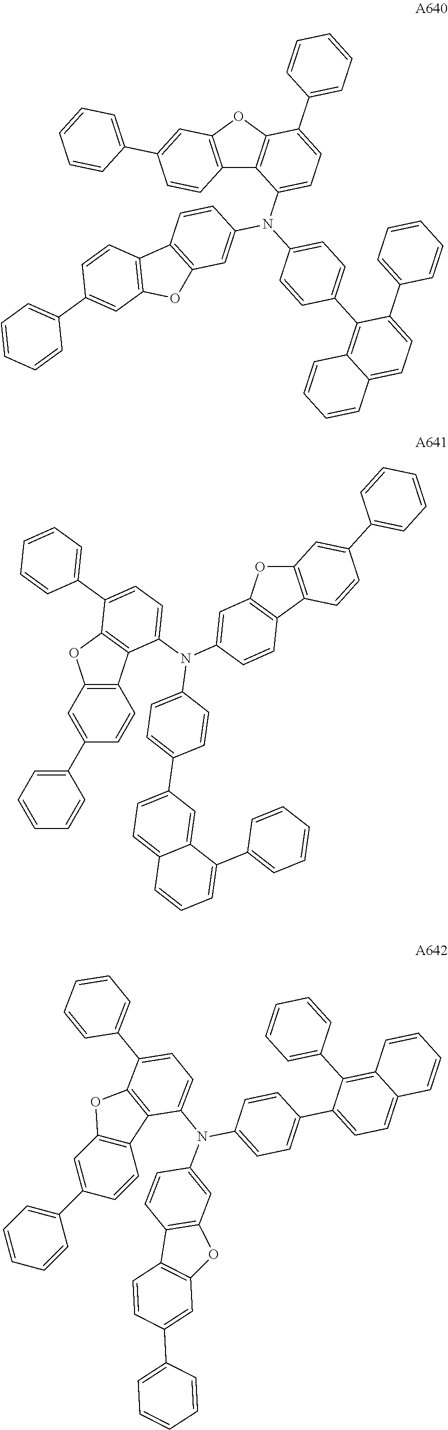 [SMILES: c1ccc(-c2ccc3c(c2)oc2cc(N(c4ccc(-c5c(-c6ccccc6)ccc6ccccc56)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6cccc(-c7ccccc7)c6c5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6ccccc6c5-c5ccccc5)cc4)c4ccc(-c5ccccc5)c5oc6cc(-c7ccccc7)ccc6c45)ccc23)cc1]

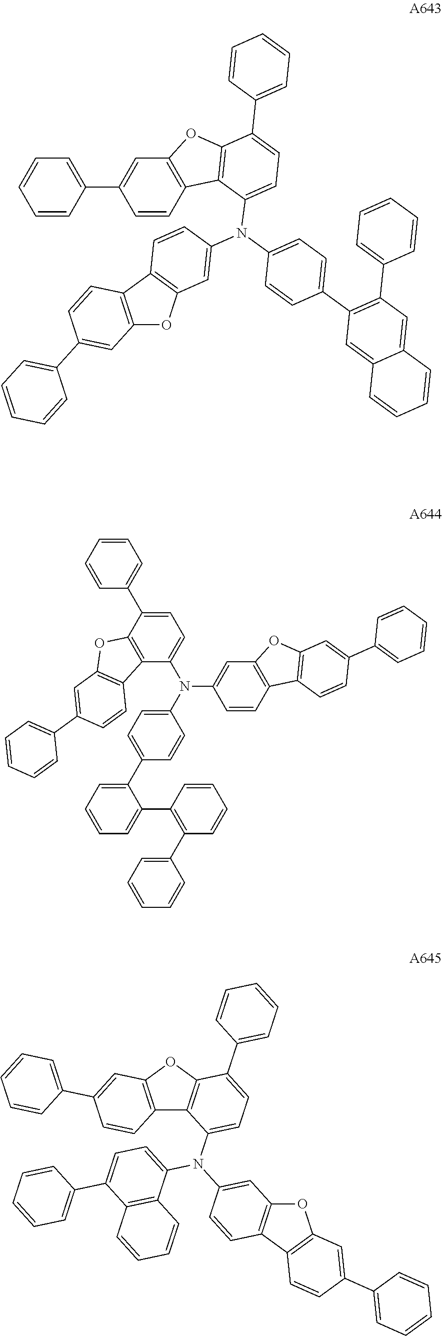 [SMILES: c1ccc(-c2ccc3c(c2)oc2cc(N(c4ccc(-c5cc6ccccc6cc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5-c5ccccc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5)c5ccccc45)c4ccc(-c5ccccc5)c5oc6cc(-c7ccccc7)ccc6c45)ccc23)cc1]

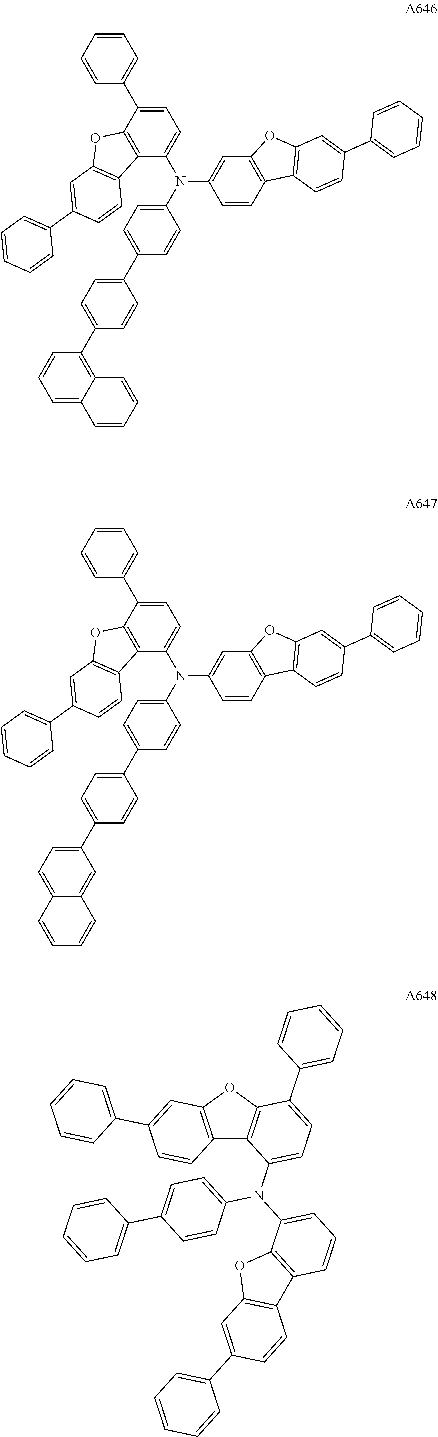 [SMILES: c1ccc(-c2ccc3c(c2)oc2cc(N(c4ccc(-c5ccc(-c6cccc7ccccc67)cc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5oc6cc(-c7ccccc7)ccc6c45)ccc23)cc1 ; c1ccc(-c2ccc(N(c3cccc4c3oc3cc(-c5ccccc5)ccc34)c3ccc(-c4ccccc4)c4oc5cc(-c6ccccc6)ccc5c34)cc2)cc1]

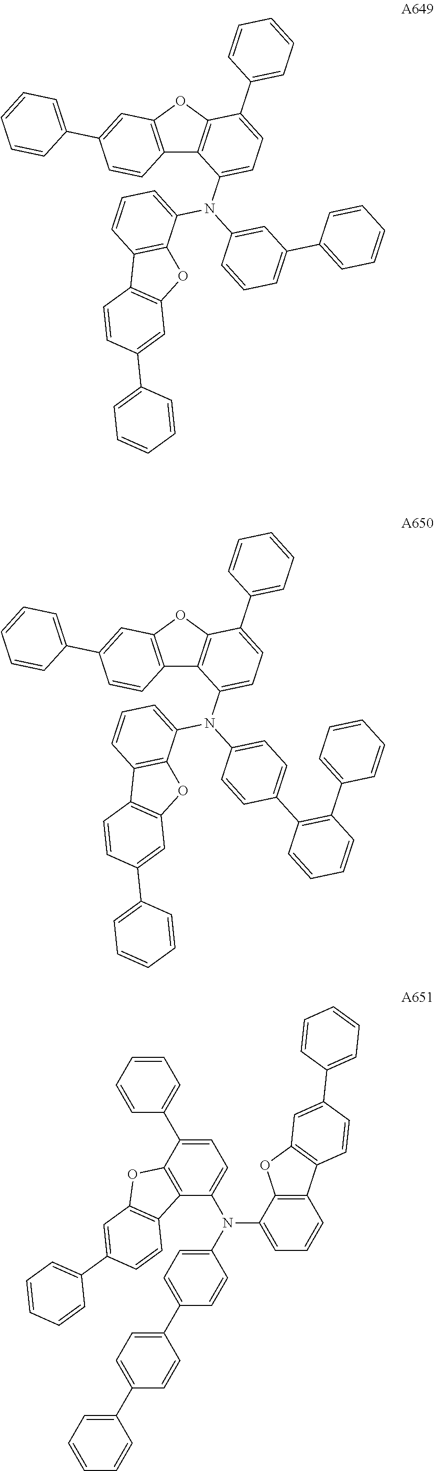 [SMILES: c1ccc(-c2cccc(N(c3cccc4c3oc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(N(c4ccc(-c5ccccc5-c5ccccc5)cc4)c4ccc(-c5ccccc5)c5oc6cc(-c7ccccc7)ccc6c45)cccc23)cc1 ; c1ccc(-c2ccc(-c3ccc(N(c4cccc5c4oc4cc(-c6ccccc6)ccc45)c4ccc(-c5ccccc5)c5oc6cc(-c7ccccc7)ccc6c45)cc3)cc2)cc1]

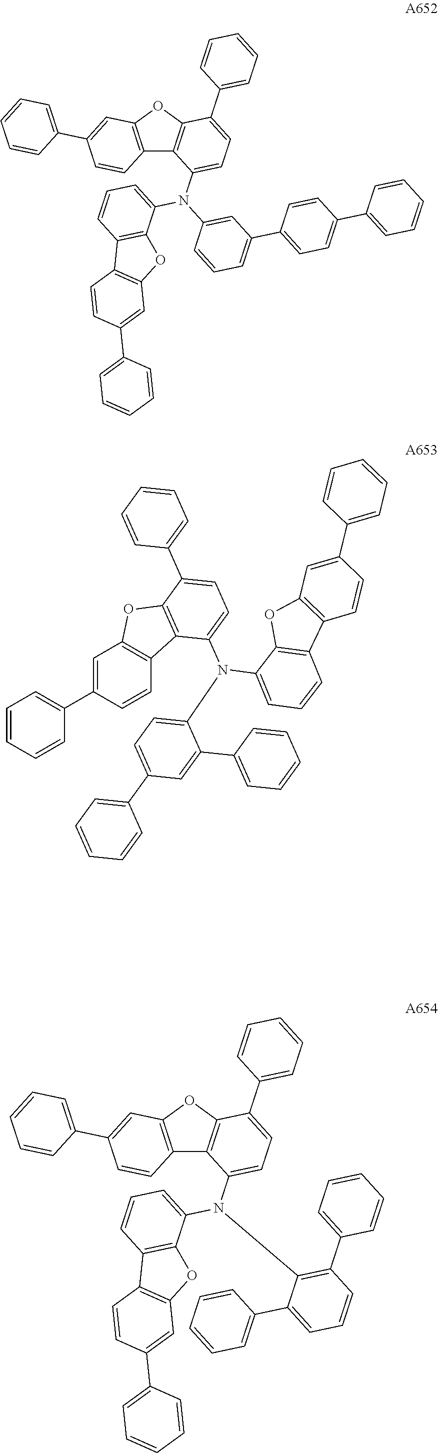 [SMILES: c1ccc(-c2ccc(-c3cccc(N(c4cccc5c4oc4cc(-c6ccccc6)ccc45)c4ccc(-c5ccccc5)c5oc6cc(-c7ccccc7)ccc6c45)c3)cc2)cc1 ; c1ccc(-c2ccc(N(c3cccc4c3oc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(N(c4c(-c5ccccc5)cccc4-c4ccccc4)c4ccc(-c5ccccc5)c5oc6cc(-c7ccccc7)ccc6c45)cccc23)cc1]

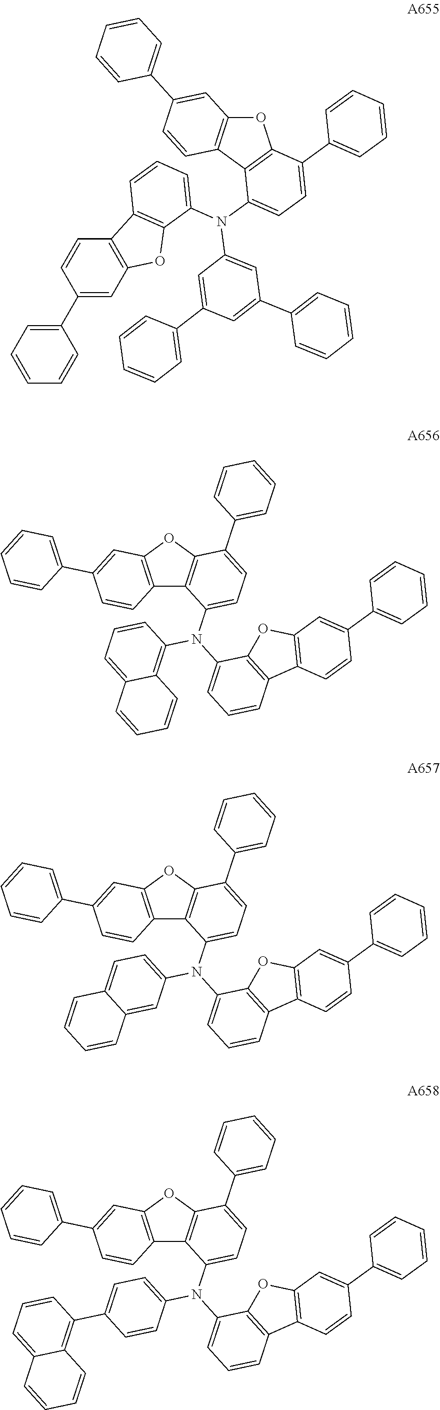 [SMILES: c1ccc(-c2cc(-c3ccccc3)cc(N(c3cccc4c3oc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(N(c4cccc5ccccc45)c4ccc(-c5ccccc5)c5oc6cc(-c7ccccc7)ccc6c45)cccc23)cc1 ; c1ccc(-c2ccc3c(c2)oc2c(N(c4ccc5ccccc5c4)c4ccc(-c5ccccc5)c5oc6cc(-c7ccccc7)ccc6c45)cccc23)cc1 ; c1ccc(-c2ccc3c(c2)oc2c(N(c4ccc(-c5cccc6ccccc56)cc4)c4ccc(-c5ccccc5)c5oc6cc(-c7ccccc7)ccc6c45)cccc23)cc1]

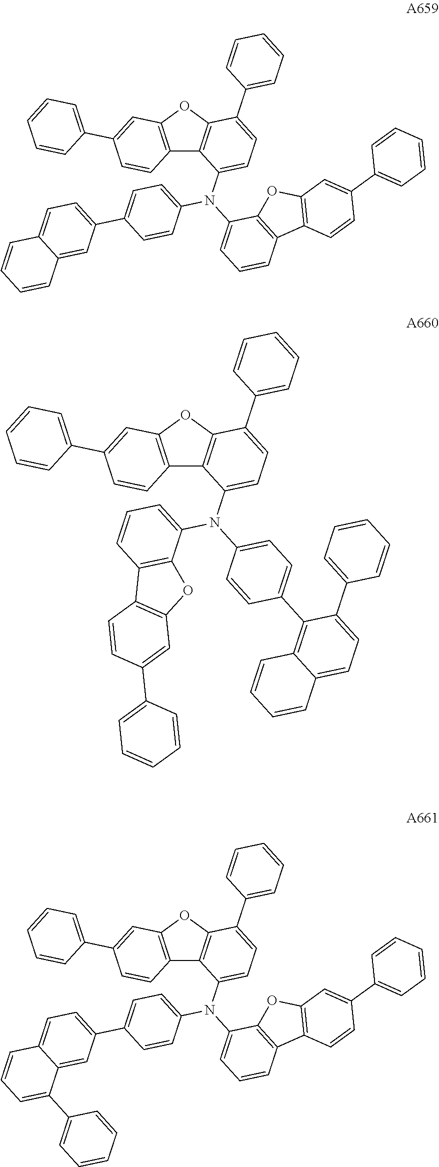 [SMILES: c1ccc(-c2ccc3c(c2)oc2c(N(c4ccc(-c5ccc6ccccc6c5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5c(-c6ccccc6)ccc6ccccc56)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6cccc(-c7ccccc7)c6c5)cc4)c4ccc(-c5ccccc5)c5oc6cc(-c7ccccc7)ccc6c45)cccc23)cc1]

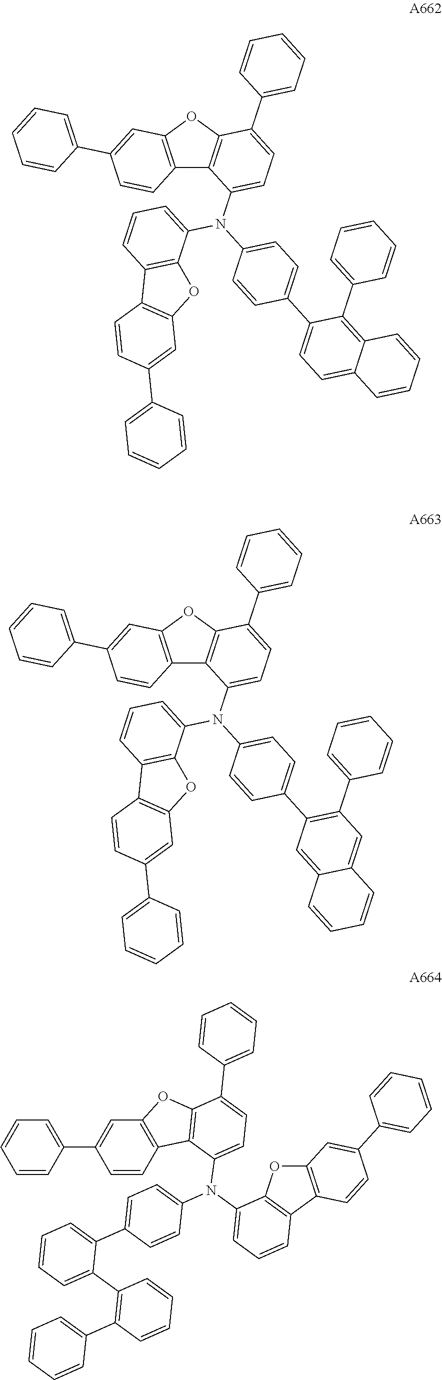 [SMILES: c1ccc(-c2ccc3c(c2)oc2c(N(c4ccc(-c5ccc6ccccc6c5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5cc6ccccc6cc5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccccc5-c5ccccc5-c5ccccc5)cc4)c4ccc(-c5ccccc5)c5oc6cc(-c7ccccc7)ccc6c45)cccc23)cc1]

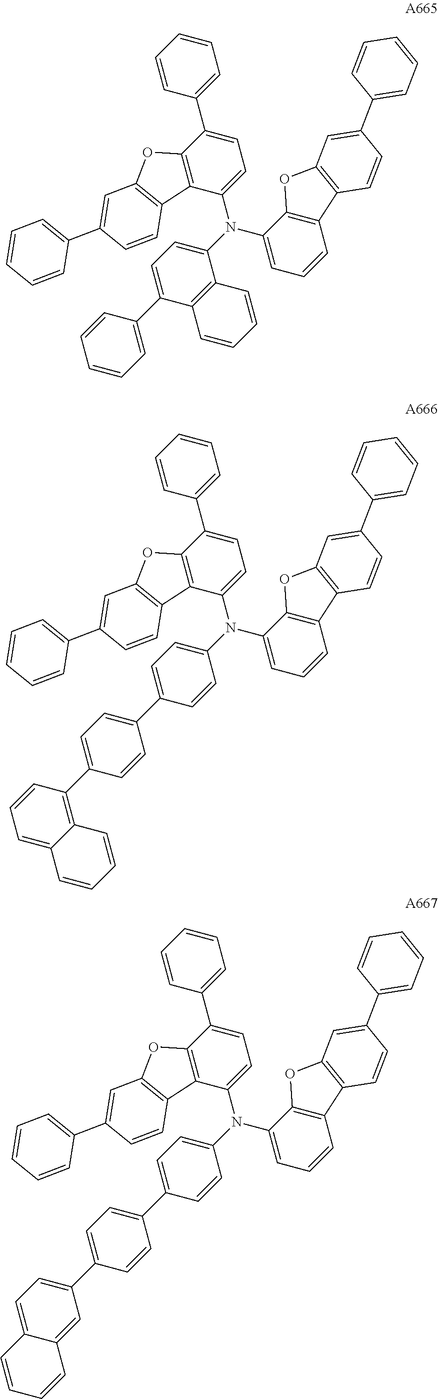 [SMILES: c1ccc(-c2ccc3c(c2)oc2c(N(c4ccc(-c5ccccc5)c5ccccc45)c4ccc(-c5ccccc5)c5oc6cc(-c7ccccc7)ccc6c45)cccc23)cc1 ; c1ccc(-c2ccc3c(c2)oc2c(N(c4ccc(-c5ccc(-c6cccc7ccccc67)cc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc(-c6ccc7ccccc7c6)cc5)cc4)c4ccc(-c5ccccc5)c5oc6cc(-c7ccccc7)ccc6c45)cccc23)cc1]

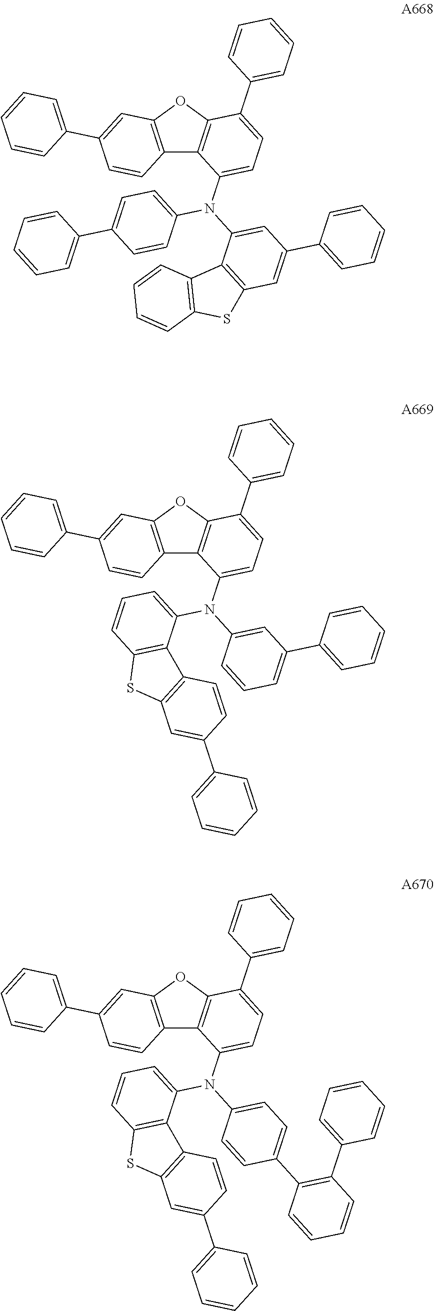 [SMILES: c1ccc(-c2ccc(N(c3cc(-c4ccccc4)cc4sc5ccccc5c34)c3ccc(-c4ccccc4)c4oc5cc(-c6ccccc6)ccc5c34)cc2)cc1 ; c1ccc(-c2cccc(N(c3cccc4sc5cc(-c6ccccc6)ccc5c34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4cccc5sc6cc(-c7ccccc7)ccc6c45)c23)cc1]

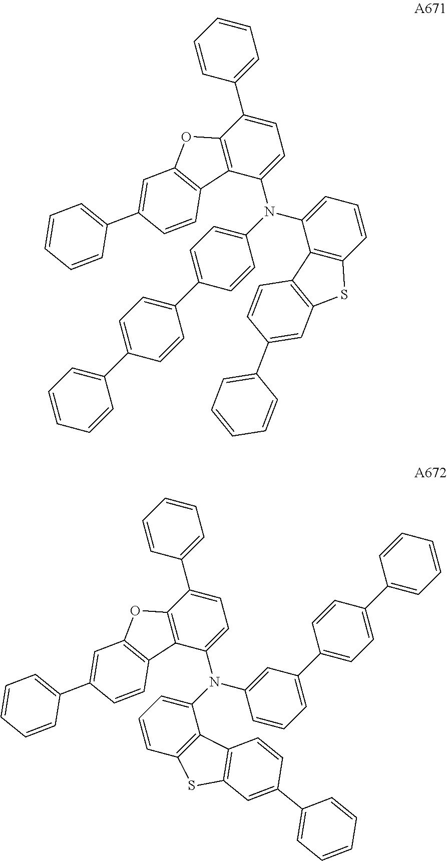 [SMILES: c1ccc(-c2ccc(-c3ccc(N(c4cccc5sc6cc(-c7ccccc7)ccc6c45)c4ccc(-c5ccccc5)c5oc6cc(-c7ccccc7)ccc6c45)cc3)cc2)cc1 ; c1ccc(-c2ccc(-c3cccc(N(c4cccc5sc6cc(-c7ccccc7)ccc6c45)c4ccc(-c5ccccc5)c5oc6cc(-c7ccccc7)ccc6c45)c3)cc2)cc1]

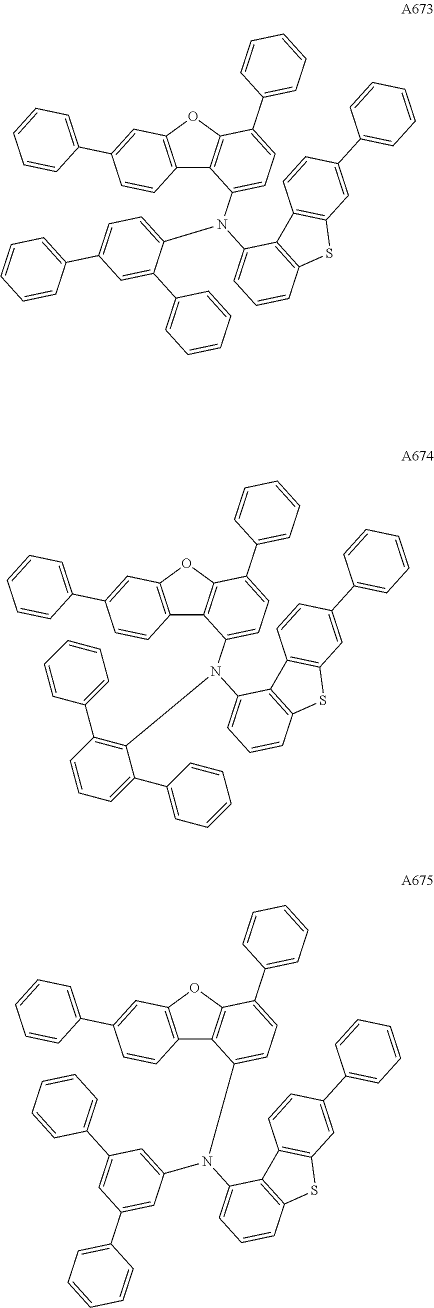 [SMILES: c1ccc(-c2ccc(N(c3cccc4sc5cc(-c6ccccc6)ccc5c34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5sc6cc(-c7ccccc7)ccc6c45)c23)cc1 ; c1ccc(-c2cc(-c3ccccc3)cc(N(c3cccc4sc5cc(-c6ccccc6)ccc5c34)c3ccc(-c4ccccc4)c4oc5cc(-c6ccccc6)ccc5c34)c2)cc1]

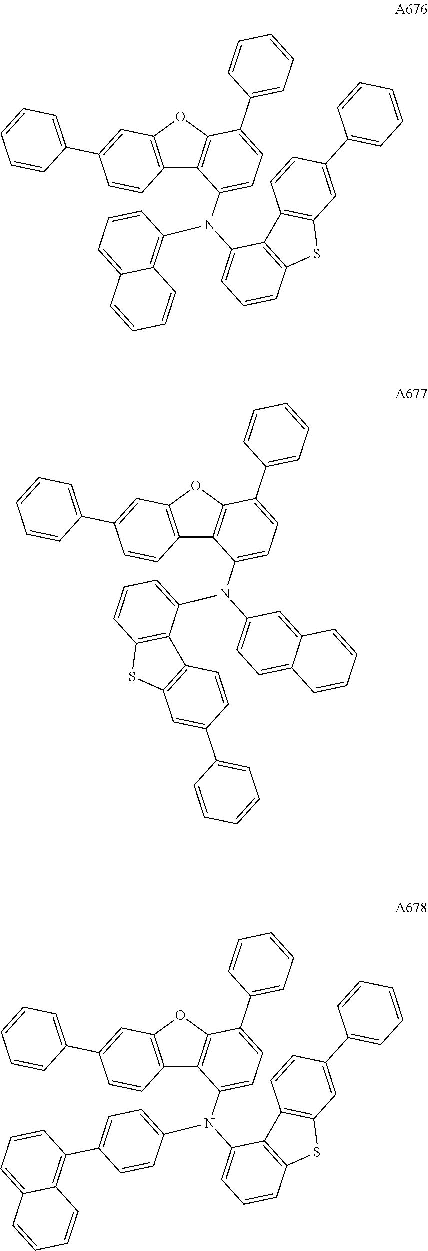 [SMILES: c1ccc(-c2ccc3c(c2)oc2c(-c4ccccc4)ccc(N(c4cccc5ccccc45)c4cccc5sc6cc(-c7ccccc7)ccc6c45)c23)cc1 ; c1ccc(-c2ccc3c(c2)oc2c(-c4ccccc4)ccc(N(c4ccc5ccccc5c4)c4cccc5sc6cc(-c7ccccc7)ccc6c45)c23)cc1 ; c1ccc(-c2ccc3c(c2)oc2c(-c4ccccc4)ccc(N(c4ccc(-c5cccc6ccccc56)cc4)c4cccc5sc6cc(-c7ccccc7)ccc6c45)c23)cc1]

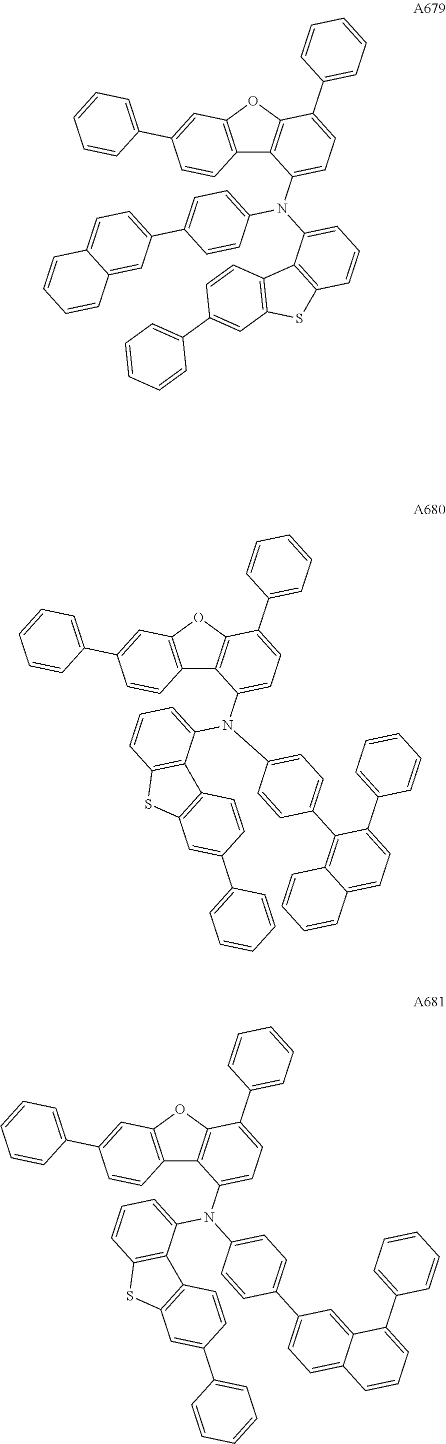 [SMILES: c1ccc(-c2ccc3c(c2)oc2c(-c4ccccc4)ccc(N(c4ccc(-c5ccc6ccccc6c5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5c(-c6ccccc6)ccc6ccccc56)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6cccc(-c7ccccc7)c6c5)cc4)c4cccc5sc6cc(-c7ccccc7)ccc6c45)c23)cc1]

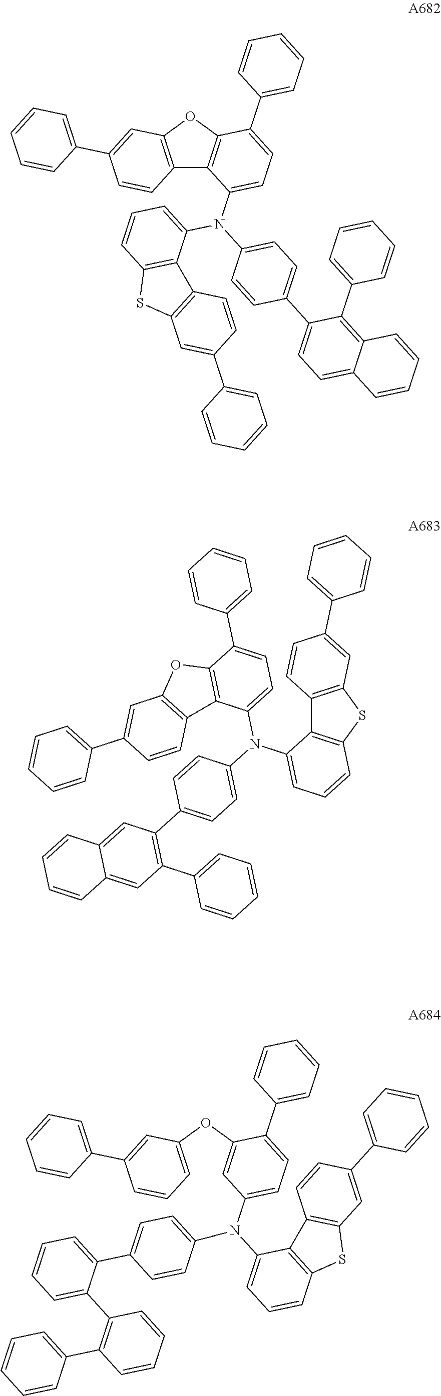 [SMILES: c1ccc(-c2ccc3c(c2)oc2c(-c4ccccc4)ccc(N(c4ccc(-c5ccc6ccccc6c5-c5ccccc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5cc6ccccc6cc5-c5ccccc5)cc4)c4cccc5sc6cc(-c7ccccc7)ccc6c45)c23)cc1 ; c1ccc(-c2cccc(Oc3cc(N(c4ccc(-c5ccccc5-c5ccccc5-c5ccccc5)cc4)c4cccc5sc6cc(-c7ccccc7)ccc6c45)ccc3-c3ccccc3)c2)cc1]

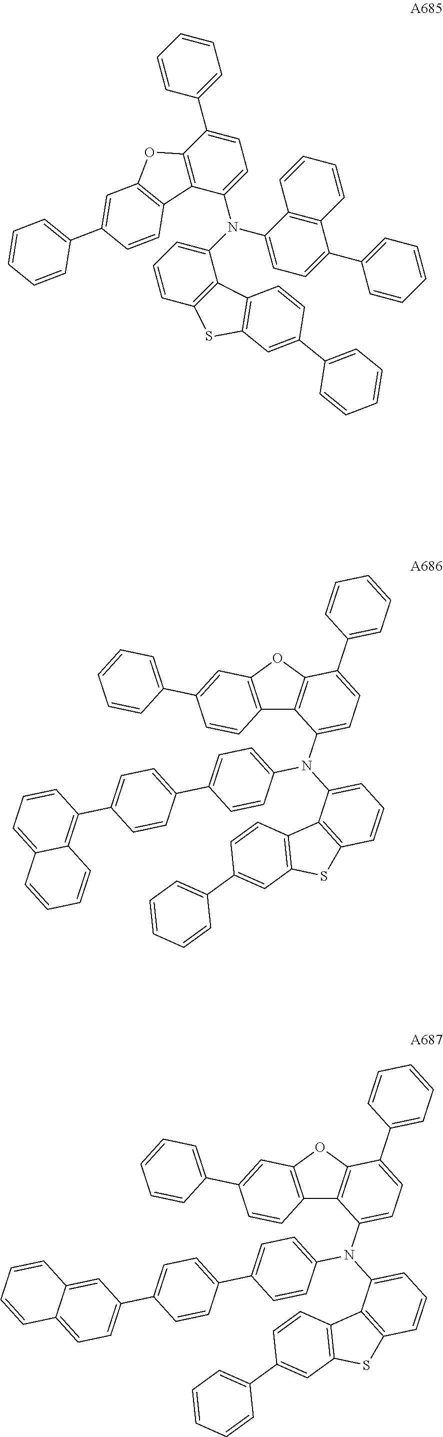 [SMILES: c1ccc(-c2ccc3c(c2)oc2c(-c4ccccc4)ccc(N(c4ccc(-c5ccccc5)c5ccccc45)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6cccc7ccccc67)cc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6ccc7ccccc7c6)cc5)cc4)c4cccc5sc6cc(-c7ccccc7)ccc6c45)c23)cc1]

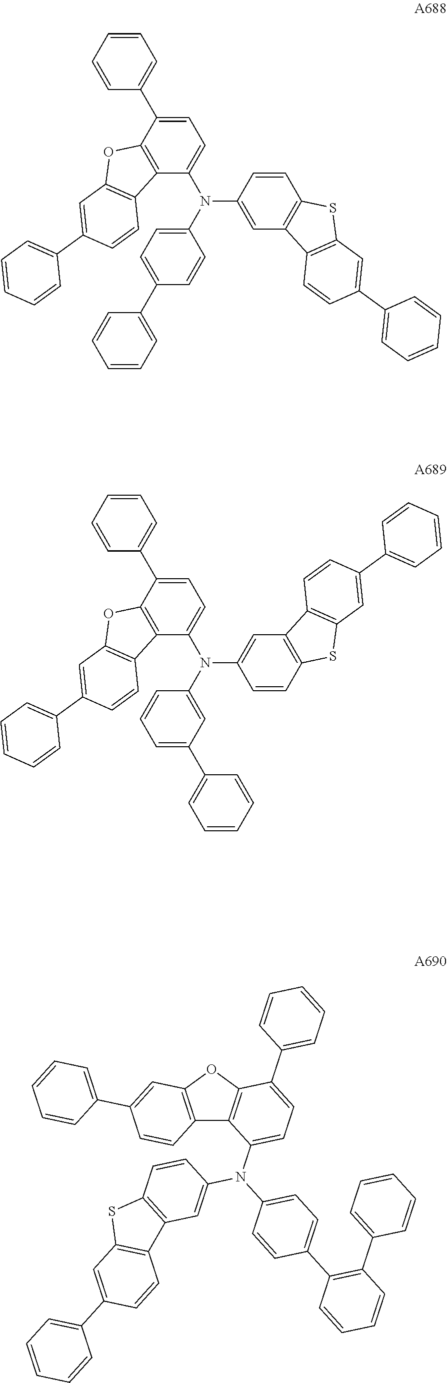 [SMILES: c1ccc(-c2ccc(N(c3ccc4sc5cc(-c6ccccc6)ccc5c4c3)c3ccc(-c4ccccc4)c4oc5cc(-c6ccccc6)ccc5c34)cc2)cc1 ; c1ccc(-c2cccc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4ccc5sc6cc(-c7ccccc7)ccc6c5c4)c23)cc1]

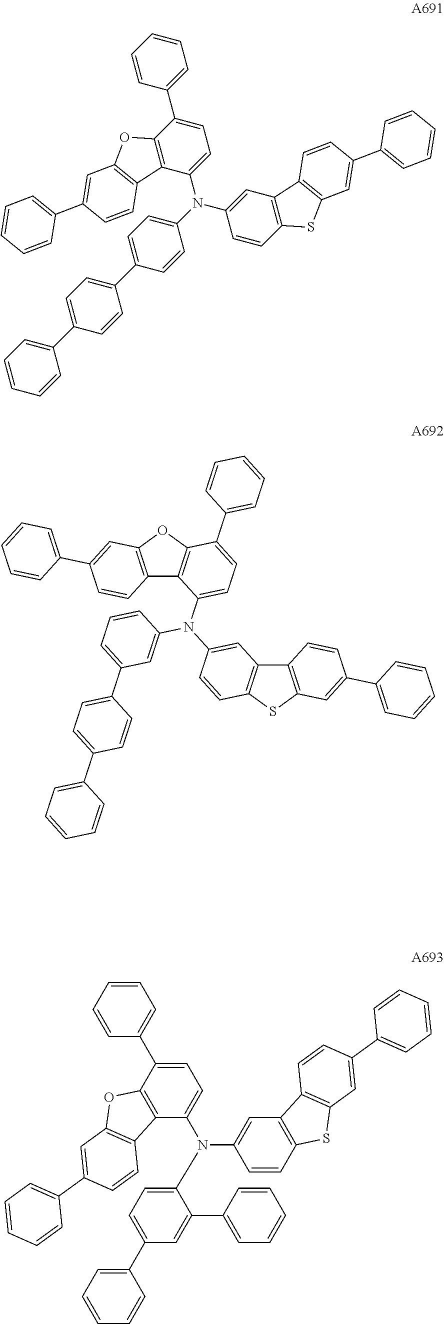 [SMILES: c1ccc(-c2ccc(-c3ccc(N(c4ccc5sc6cc(-c7ccccc7)ccc6c5c4)c4ccc(-c5ccccc5)c5oc6cc(-c7ccccc7)ccc6c45)cc3)cc2)cc1 ; c1ccc(-c2ccc(-c3cccc(N(c4ccc5sc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4sc5cc(-c6ccccc6)ccc5c4c3)c3ccc(-c4ccccc4)c4oc5cc(-c6ccccc6)ccc5c34)c(-c3ccccc3)c2)cc1]

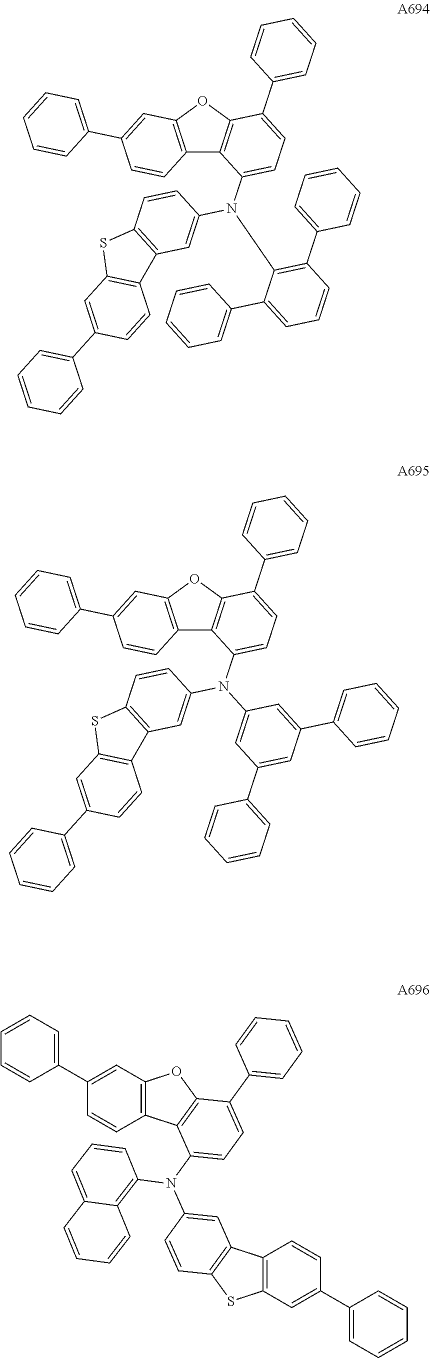 [SMILES: c1ccc(-c2ccc3c(c2)oc2c(-c4ccccc4)ccc(N(c4ccc5sc6cc(-c7ccccc7)ccc6c5c4)c4c(-c5ccccc5)cccc4-c4ccccc4)c23)cc1 ; c1ccc(-c2cc(-c3ccccc3)cc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5sc6cc(-c7ccccc7)ccc6c5c4)c4cccc5ccccc45)c23)cc1]

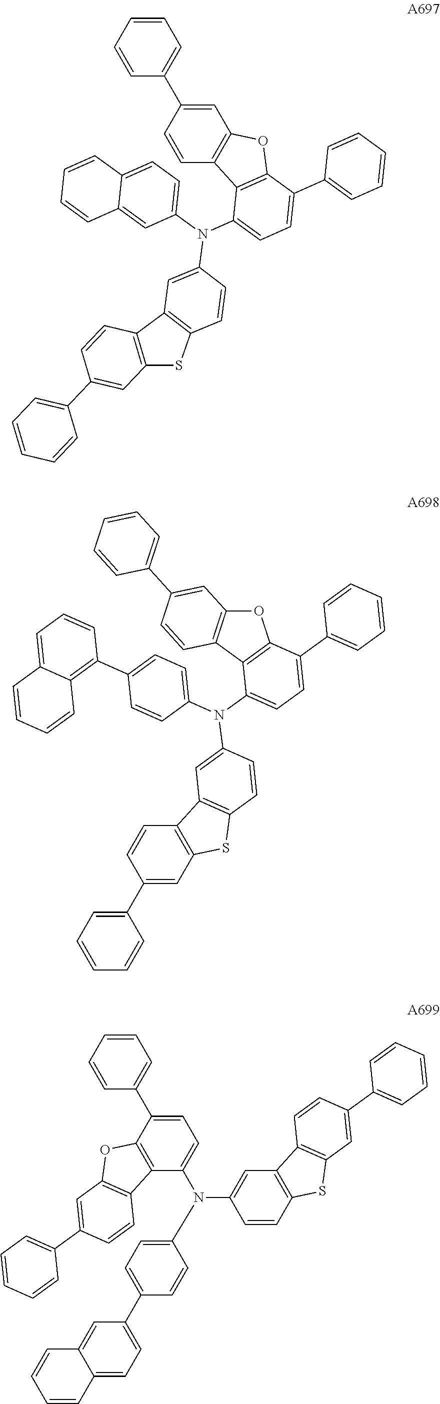 [SMILES: c1ccc(-c2ccc3c(c2)oc2c(-c4ccccc4)ccc(N(c4ccc5ccccc5c4)c4ccc5sc6cc(-c7ccccc7)ccc6c5c4)c23)cc1 ; c1ccc(-c2ccc3c(c2)oc2c(-c4ccccc4)ccc(N(c4ccc(-c5cccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5)cc4)c4ccc5sc6cc(-c7ccccc7)ccc6c5c4)c23)cc1]

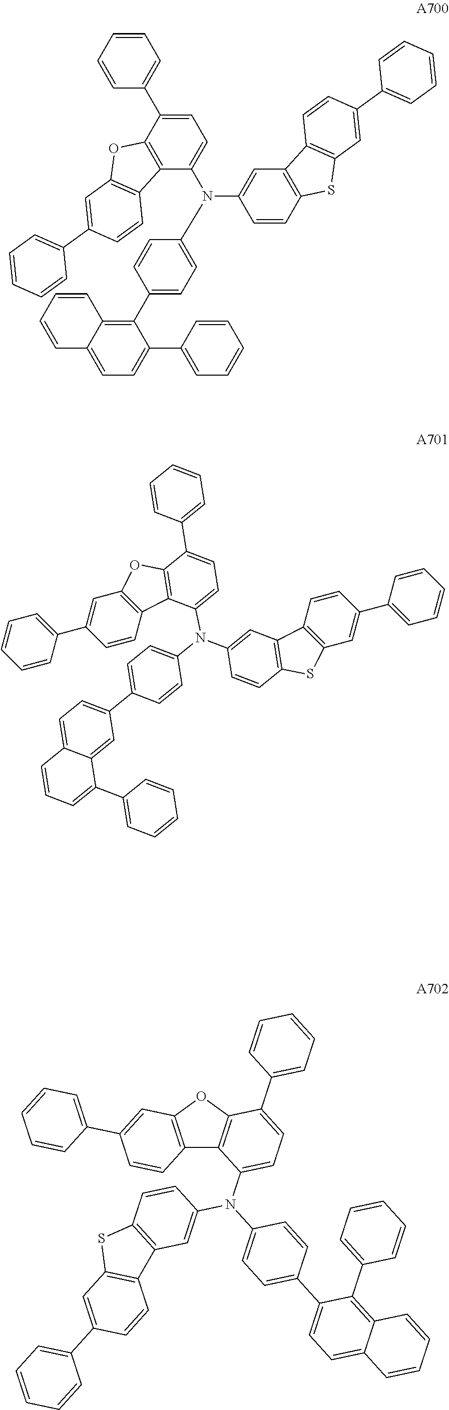 [SMILES: c1ccc(-c2ccc3c(c2)oc2c(-c4ccccc4)ccc(N(c4ccc(-c5c(-c6ccccc6)ccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6cccc(-c7ccccc7)c6c5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5-c5ccccc5)cc4)c4ccc5sc6cc(-c7ccccc7)ccc6c5c4)c23)cc1]

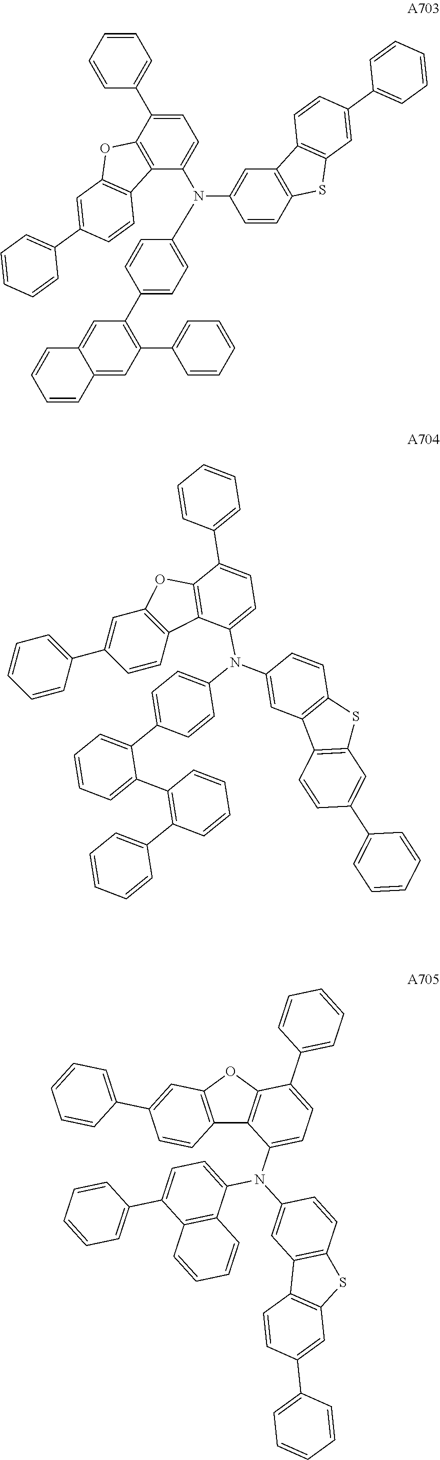 [SMILES: c1ccc(-c2ccc3c(c2)oc2c(-c4ccccc4)ccc(N(c4ccc(-c5cc6ccccc6cc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccccc5-c5ccccc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc5sc6cc(-c7ccccc7)ccc6c5c4)c4ccc(-c5ccccc5)c5ccccc45)c23)cc1]

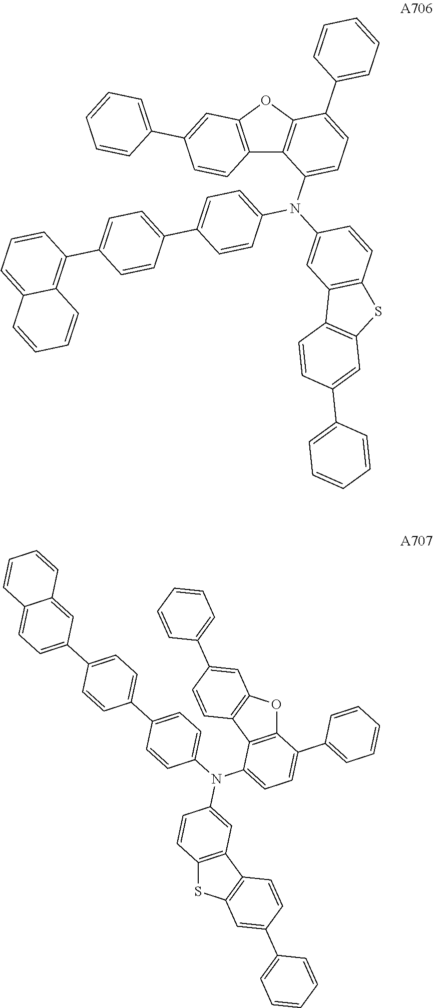 [SMILES: c1ccc(-c2ccc3c(c2)oc2c(-c4ccccc4)ccc(N(c4ccc(-c5ccc(-c6cccc7ccccc67)cc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5sc6cc(-c7ccccc7)ccc6c5c4)c23)cc1]

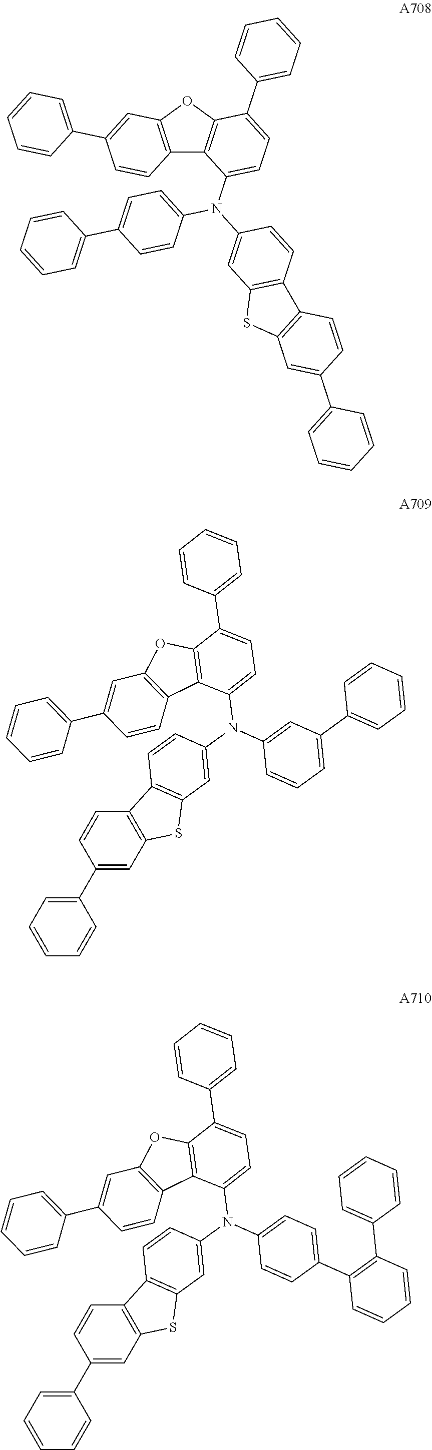 [SMILES: c1ccc(-c2ccc(N(c3ccc4c(c3)sc3cc(-c5ccccc5)ccc34)c3ccc(-c4ccccc4)c4oc5cc(-c6ccccc6)ccc5c34)cc2)cc1 ; c1ccc(-c2cccc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4ccc5c(c4)sc4cc(-c6ccccc6)ccc45)c23)cc1]

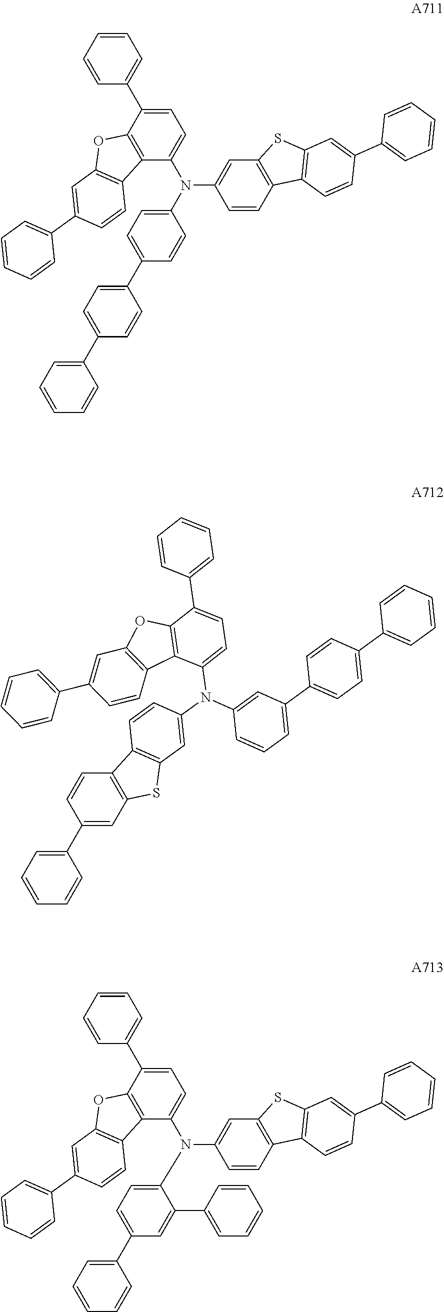 [SMILES: c1ccc(-c2ccc(-c3ccc(N(c4ccc5c(c4)sc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)sc4cc(-c6ccccc6)ccc45)c4ccc(-c5ccccc5)c5oc6cc(-c7ccccc7)ccc6c45)c3)cc2)cc1 ; c1ccc(-c2ccc(N(c3ccc4c(c3)sc3cc(-c5ccccc5)ccc34)c3ccc(-c4ccccc4)c4oc5cc(-c6ccccc6)ccc5c34)c(-c3ccccc3)c2)cc1]

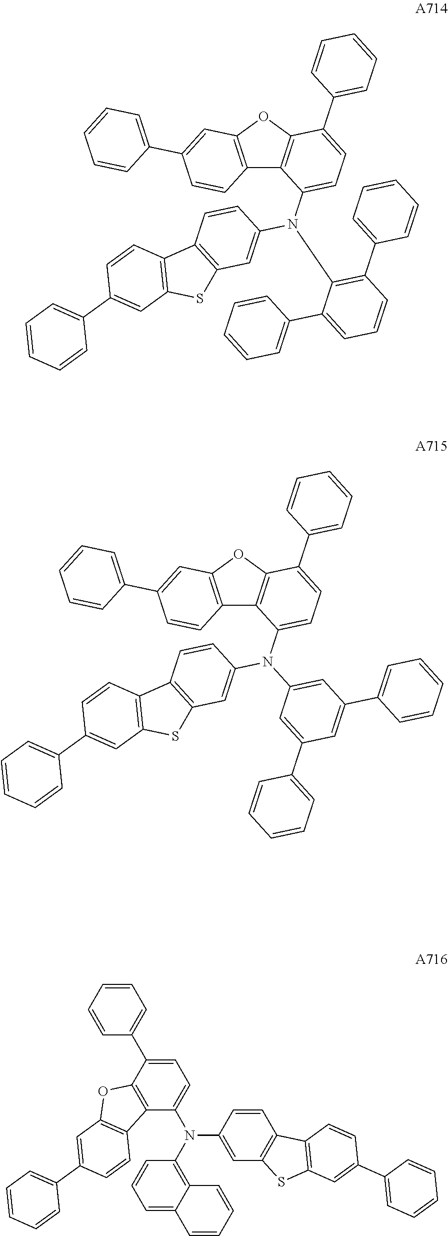 [SMILES: c1ccc(-c2ccc3c(c2)oc2c(-c4ccccc4)ccc(N(c4ccc5c(c4)sc4cc(-c6ccccc6)ccc45)c4c(-c5ccccc5)cccc4-c4ccccc4)c23)cc1 ; c1ccc(-c2cc(-c3ccccc3)cc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5c(c4)sc4cc(-c6ccccc6)ccc45)c4cccc5ccccc45)c23)cc1]

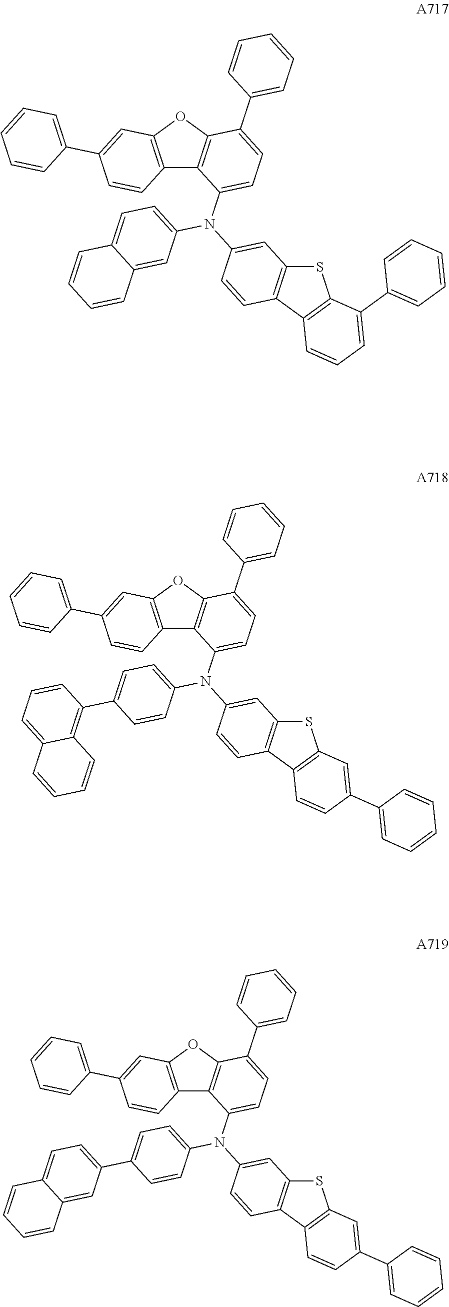 [SMILES: c1ccc(-c2ccc3c(c2)oc2c(-c4ccccc4)ccc(N(c4ccc5ccccc5c4)c4ccc5c(c4)sc4c(-c6ccccc6)cccc45)c23)cc1 ; c1ccc(-c2ccc3c(c2)oc2c(-c4ccccc4)ccc(N(c4ccc(-c5cccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5)cc4)c4ccc5c(c4)sc4cc(-c6ccccc6)ccc45)c23)cc1]

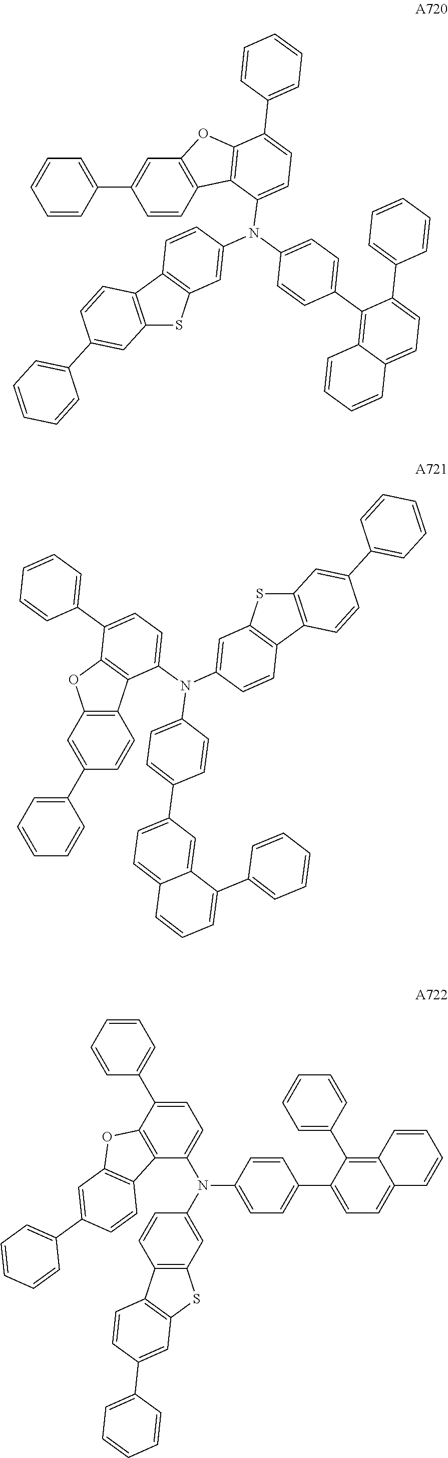 [SMILES: c1ccc(-c2ccc3c(c2)oc2c(-c4ccccc4)ccc(N(c4ccc(-c5c(-c6ccccc6)ccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6cccc(-c7ccccc7)c6c5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5-c5ccccc5)cc4)c4ccc5c(c4)sc4cc(-c6ccccc6)ccc45)c23)cc1]

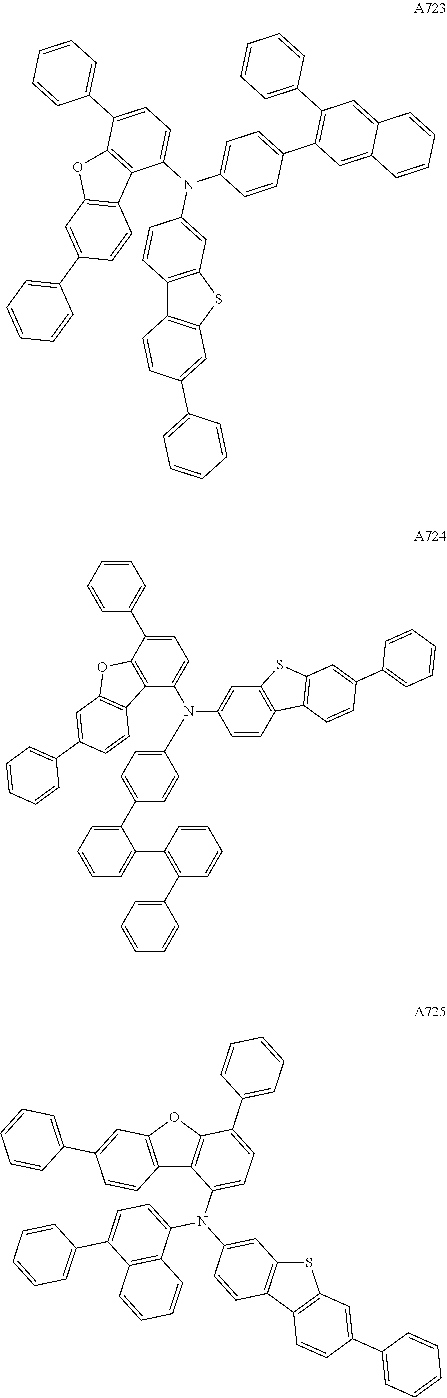 [SMILES: c1ccc(-c2ccc3c(c2)oc2c(-c4ccccc4)ccc(N(c4ccc(-c5cc6ccccc6cc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccccc5-c5ccccc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc5c(c4)sc4cc(-c6ccccc6)ccc45)c4ccc(-c5ccccc5)c5ccccc45)c23)cc1]

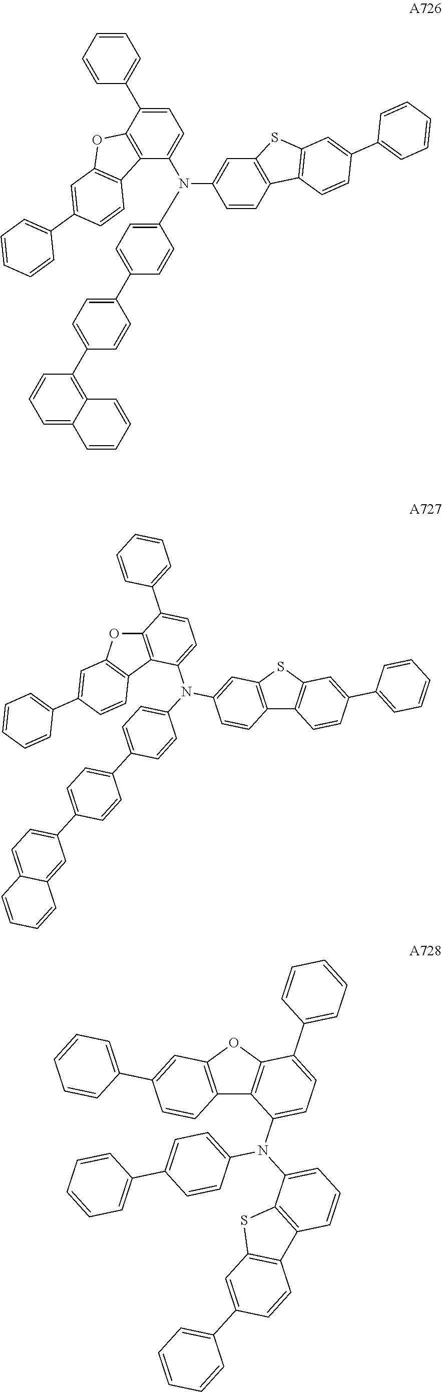 [SMILES: c1ccc(-c2ccc3c(c2)oc2c(-c4ccccc4)ccc(N(c4ccc(-c5ccc(-c6cccc7ccccc67)cc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5c(c4)sc4cc(-c6ccccc6)ccc45)c23)cc1 ; c1ccc(-c2ccc(N(c3cccc4c3sc3cc(-c5ccccc5)ccc34)c3ccc(-c4ccccc4)c4oc5cc(-c6ccccc6)ccc5c34)cc2)cc1]

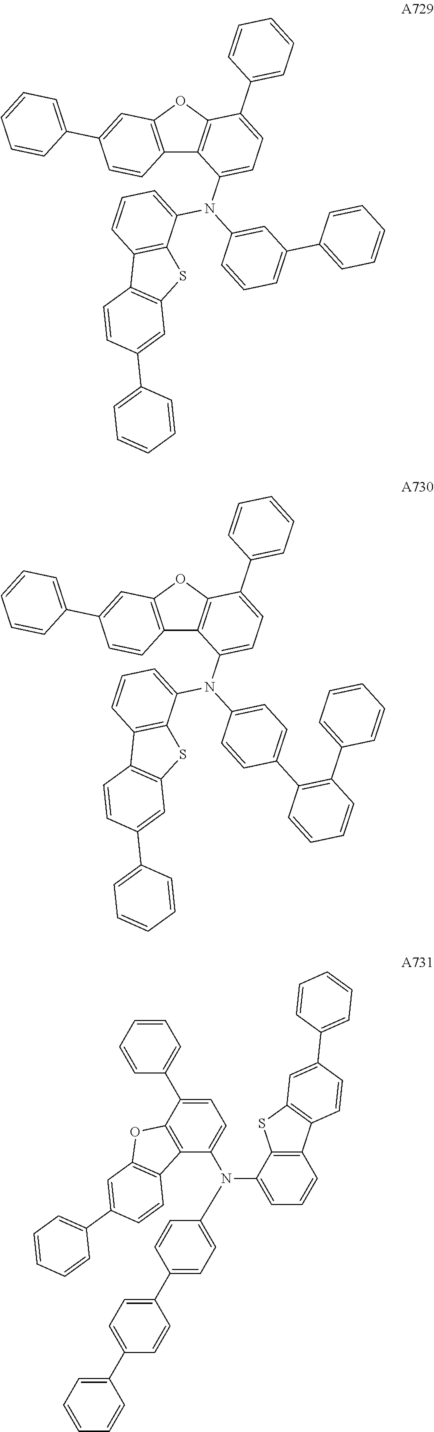 [SMILES: c1ccc(-c2cccc(N(c3cccc4c3sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4cccc5c4sc4cc(-c6ccccc6)ccc45)c23)cc1 ; c1ccc(-c2ccc(-c3ccc(N(c4cccc5c4sc4cc(-c6ccccc6)ccc45)c4ccc(-c5ccccc5)c5oc6cc(-c7ccccc7)ccc6c45)cc3)cc2)cc1]

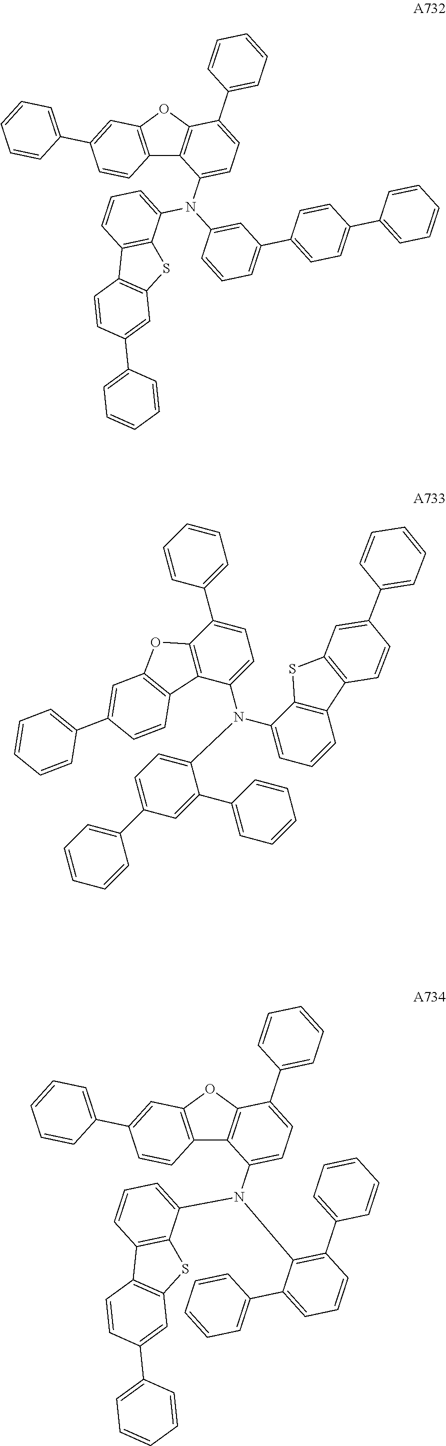 [SMILES: c1ccc(-c2ccc(-c3cccc(N(c4cccc5c4sc4cc(-c6ccccc6)ccc45)c4ccc(-c5ccccc5)c5oc6cc(-c7ccccc7)ccc6c45)c3)cc2)cc1 ; c1ccc(-c2ccc(N(c3cccc4c3sc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5c4sc4cc(-c6ccccc6)ccc45)c23)cc1]

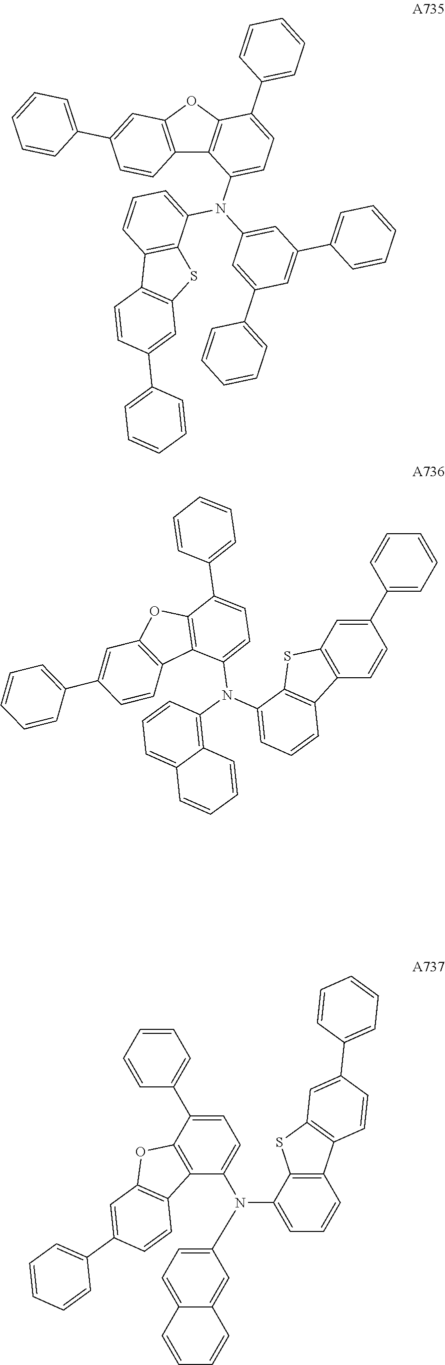 [SMILES: c1ccc(-c2cc(-c3ccccc3)cc(N(c3cccc4c3sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4cccc5ccccc45)c4cccc5c4sc4cc(-c6ccccc6)ccc45)c23)cc1 ; c1ccc(-c2ccc3c(c2)oc2c(-c4ccccc4)ccc(N(c4ccc5ccccc5c4)c4cccc5c4sc4cc(-c6ccccc6)ccc45)c23)cc1]

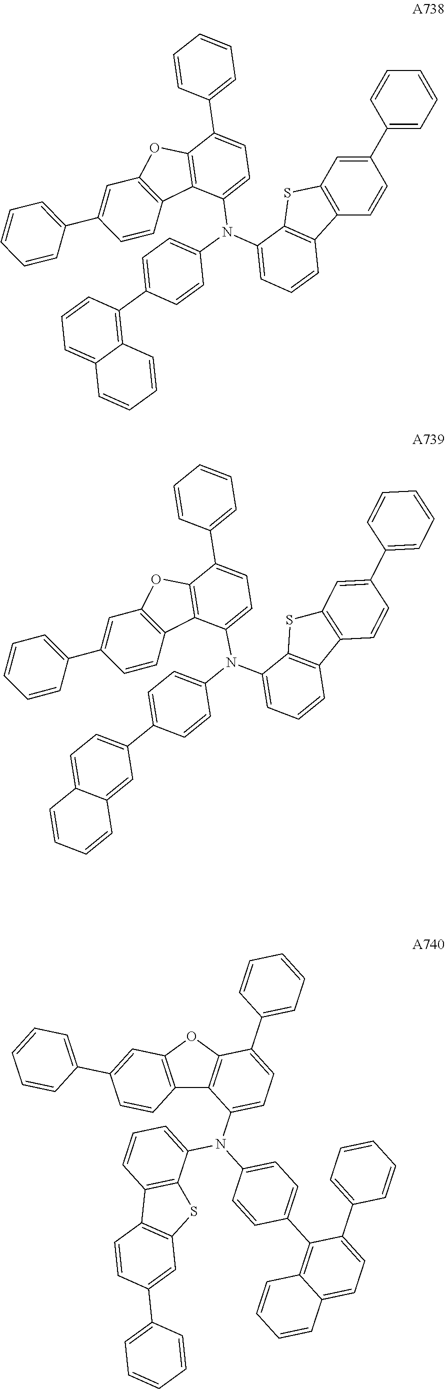 [SMILES: c1ccc(-c2ccc3c(c2)oc2c(-c4ccccc4)ccc(N(c4ccc(-c5cccc6ccccc56)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6ccccc6c5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5c(-c6ccccc6)ccc6ccccc56)cc4)c4cccc5c4sc4cc(-c6ccccc6)ccc45)c23)cc1]

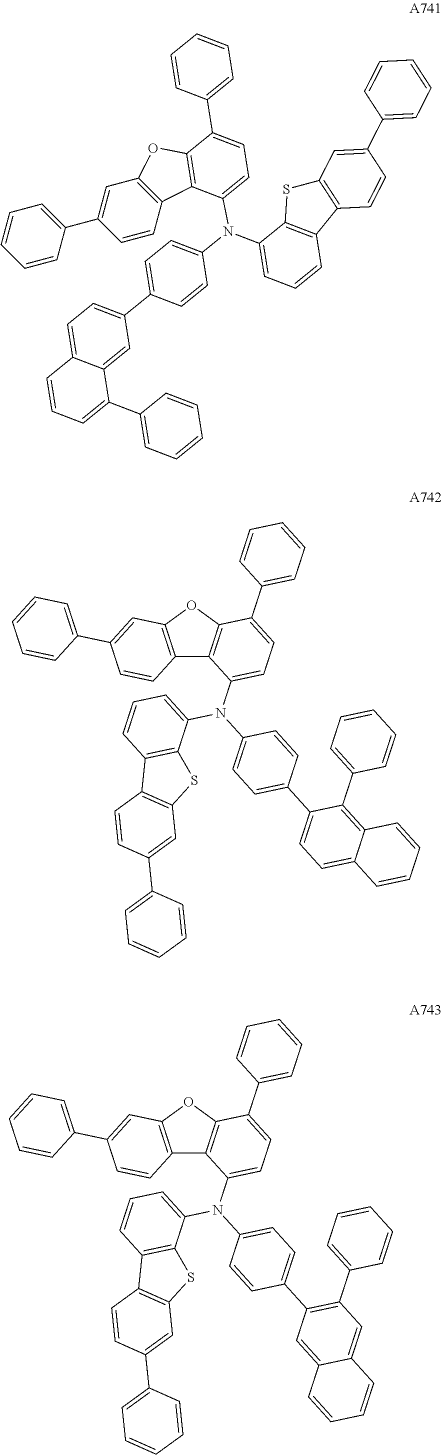 [SMILES: c1ccc(-c2ccc3c(c2)oc2c(-c4ccccc4)ccc(N(c4ccc(-c5ccc6cccc(-c7ccccc7)c6c5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6ccccc6c5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5cc6ccccc6cc5-c5ccccc5)cc4)c4cccc5c4sc4cc(-c6ccccc6)ccc45)c23)cc1]

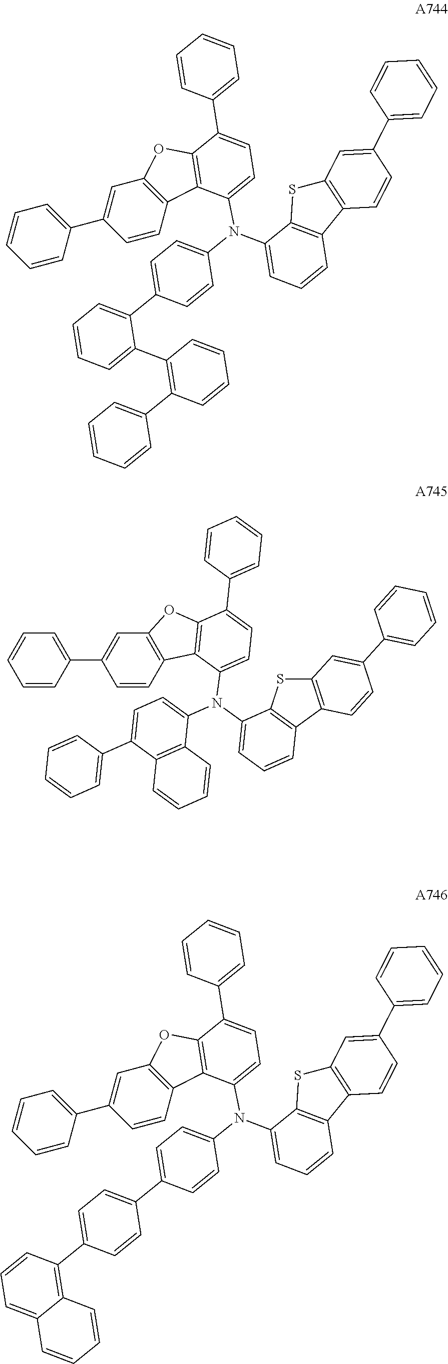 [SMILES: c1ccc(-c2ccc3c(c2)oc2c(-c4ccccc4)ccc(N(c4ccc(-c5ccccc5-c5ccccc5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccccc5)c5ccccc45)c4cccc5c4sc4cc(-c6ccccc6)ccc45)c23)cc1 ; c1ccc(-c2ccc3c(c2)oc2c(-c4ccccc4)ccc(N(c4ccc(-c5ccc(-c6cccc7ccccc67)cc5)cc4)c4cccc5c4sc4cc(-c6ccccc6)ccc45)c23)cc1]

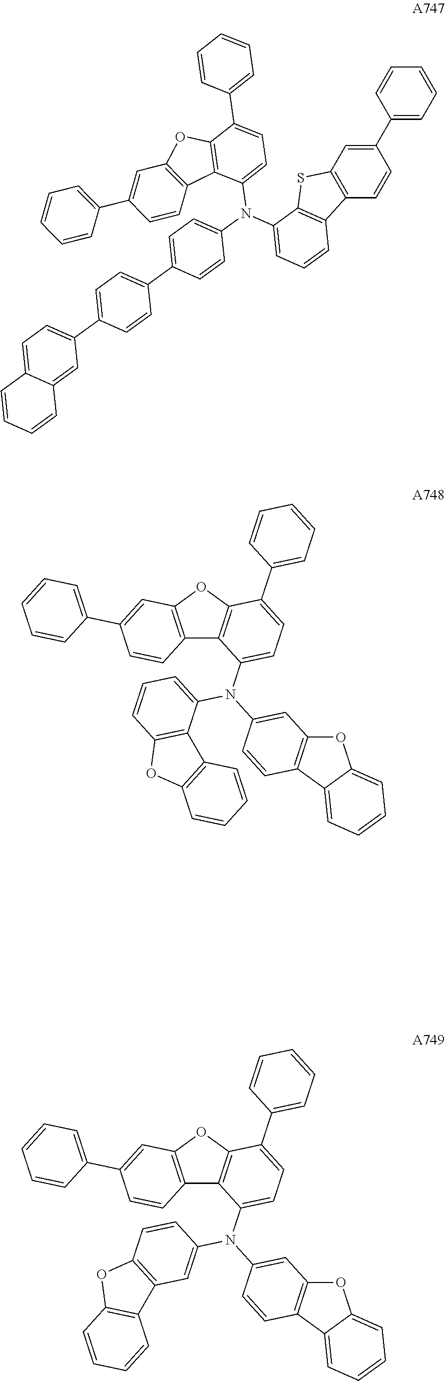 [SMILES: c1ccc(-c2ccc3c(c2)oc2c(-c4ccccc4)ccc(N(c4ccc(-c5ccc(-c6ccc7ccccc7c6)cc5)cc4)c4cccc5c4sc4cc(-c6ccccc6)ccc45)c23)cc1 ; c1ccc(-c2ccc3c(c2)oc2c(-c4ccccc4)ccc(N(c4ccc5c(c4)oc4ccccc45)c4cccc5oc6ccccc6c45)c23)cc1 ; c1ccc(-c2ccc3c(c2)oc2c(-c4ccccc4)ccc(N(c4ccc5c(c4)oc4ccccc45)c4ccc5oc6ccccc6c5c4)c23)cc1]

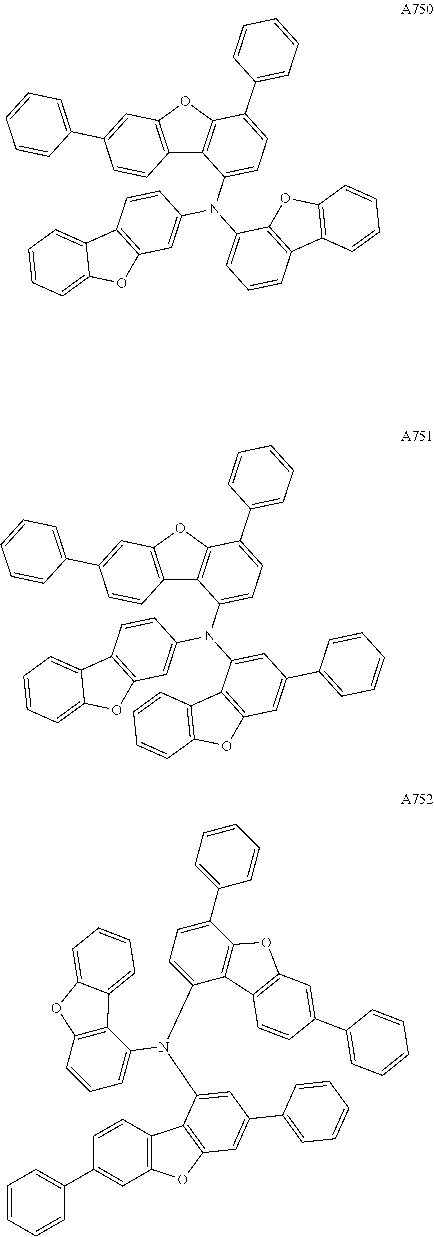 [SMILES: c1ccc(-c2ccc3c(c2)oc2c(-c4ccccc4)ccc(N(c4ccc5c(c4)oc4ccccc45)c4cccc5c4oc4ccccc45)c23)cc1 ; c1ccc(-c2ccc3c(c2)oc2c(-c4ccccc4)ccc(N(c4ccc5c(c4)oc4ccccc45)c4cc(-c5ccccc5)cc5oc6ccccc6c45)c23)cc1 ; c1ccc(-c2ccc3c(c2)oc2cc(-c4ccccc4)cc(N(c4cccc5oc6ccccc6c45)c4ccc(-c5ccccc5)c5oc6cc(-c7ccccc7)ccc6c45)c23)cc1]

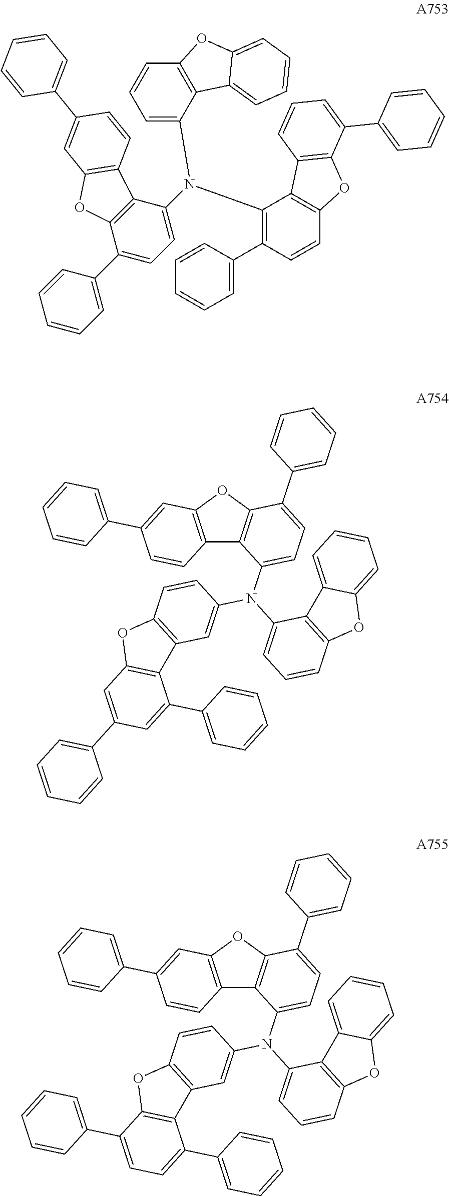 [SMILES: c1ccc(-c2ccc3c(c2)oc2c(-c4ccccc4)ccc(N(c4cccc5oc6ccccc6c45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5oc6ccccc6c45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5oc6ccccc6c45)c23)cc1]

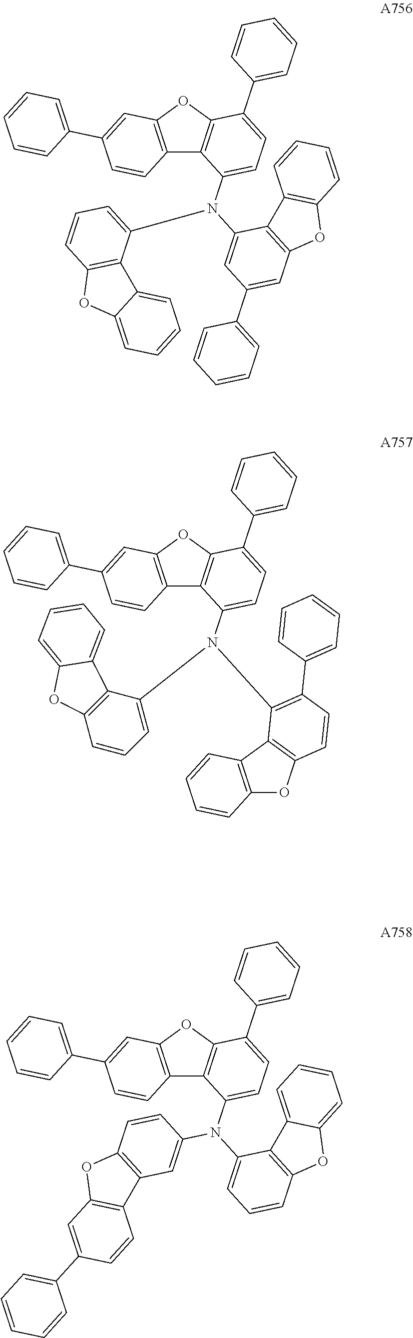 [SMILES: c1ccc(-c2ccc3c(c2)oc2c(-c4ccccc4)ccc(N(c4cccc5oc6ccccc6c45)c4cc(-c5ccccc5)cc5oc6ccccc6c45)c23)cc1 ; c1ccc(-c2ccc3c(c2)oc2c(-c4ccccc4)ccc(N(c4cccc5oc6ccccc6c45)c4c(-c5ccccc5)ccc5oc6ccccc6c45)c23)cc1 ; c1ccc(-c2ccc3c(c2)oc2ccc(N(c4cccc5oc6ccccc6c45)c4ccc(-c5ccccc5)c5oc6cc(-c7ccccc7)ccc6c45)cc23)cc1]

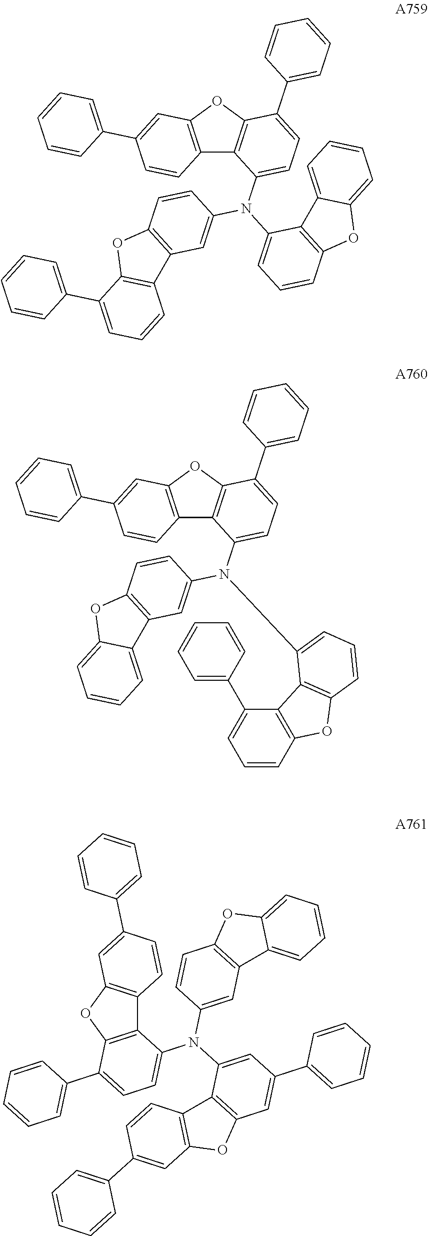 [SMILES: c1ccc(-c2ccc3c(c2)oc2c(-c4ccccc4)ccc(N(c4ccc5oc6c(-c7ccccc7)cccc6c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5oc6ccccc6c5c4)c4ccc(-c5ccccc5)c5oc6cc(-c7ccccc7)ccc6c45)c23)cc1]

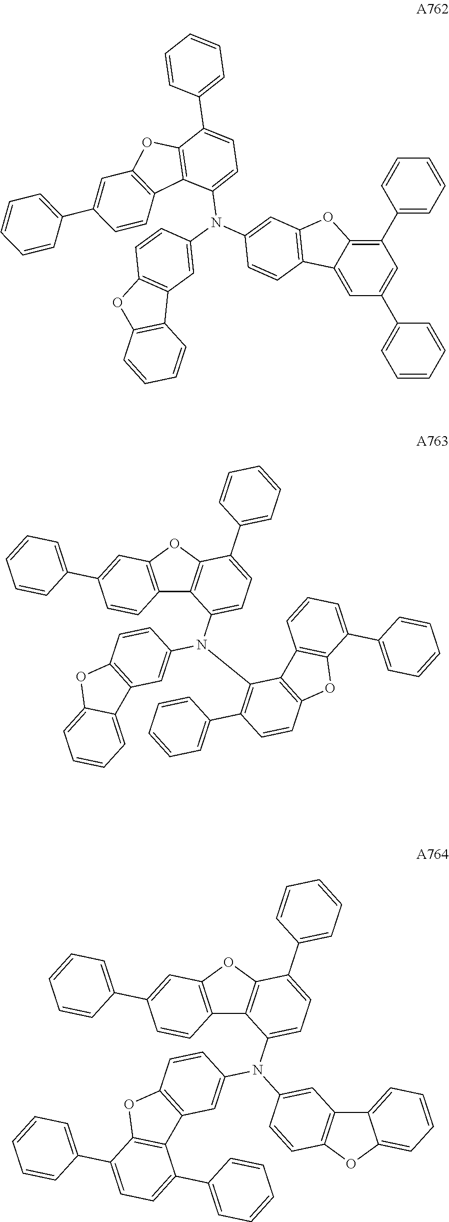 [SMILES: c1ccc(-c2ccc3c(c2)oc2c(-c4ccccc4)ccc(N(c4ccc5c(c4)oc4c(-c6ccccc6)cc(-c6ccccc6)cc45)c4ccc5oc6ccccc6c5c4)c23)cc1 ; c1ccc(-c2ccc3c(c2)oc2c(-c4ccccc4)ccc(N(c4ccc5oc6ccccc6c5c4)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6ccccc6c5c4)c4ccc5oc6c(-c7ccccc7)ccc(-c7ccccc7)c6c5c4)c23)cc1]

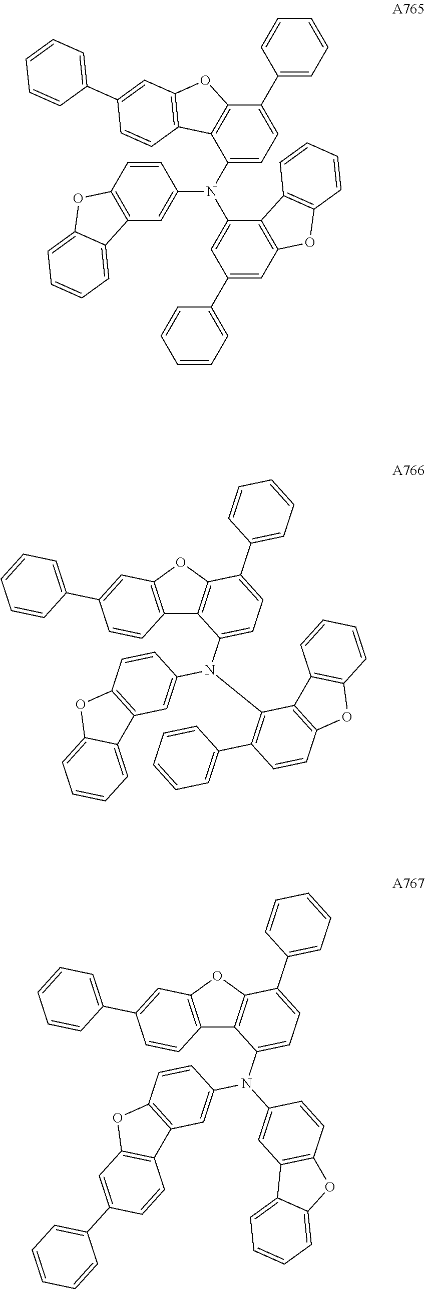 [SMILES: c1ccc(-c2ccc3c(c2)oc2c(-c4ccccc4)ccc(N(c4ccc5oc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc5oc6ccccc6c5c4)c4c(-c5ccccc5)ccc5oc6ccccc6c45)c23)cc1 ; c1ccc(-c2ccc3c(c2)oc2ccc(N(c4ccc5oc6ccccc6c5c4)c4ccc(-c5ccccc5)c5oc6cc(-c7ccccc7)ccc6c45)cc23)cc1]

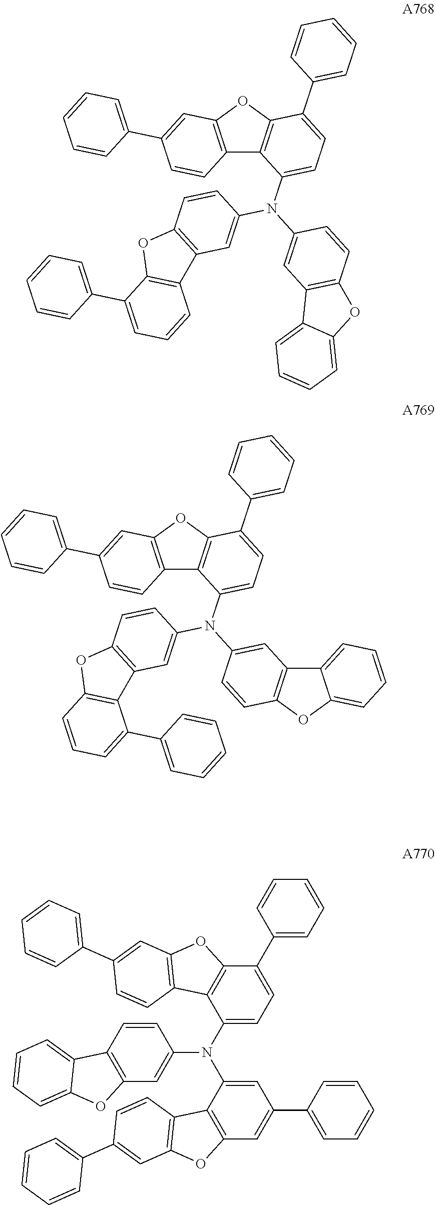 [SMILES: c1ccc(-c2ccc3c(c2)oc2c(-c4ccccc4)ccc(N(c4ccc5oc6ccccc6c5c4)c4ccc5oc6c(-c7ccccc7)cccc6c5c4)c23)cc1 ; c1ccc(-c2ccc3c(c2)oc2c(-c4ccccc4)ccc(N(c4ccc5oc6ccccc6c5c4)c4ccc5oc6cccc(-c7ccccc7)c6c5c4)c23)cc1 ; c1ccc(-c2ccc3c(c2)oc2cc(-c4ccccc4)cc(N(c4ccc5c(c4)oc4ccccc45)c4ccc(-c5ccccc5)c5oc6cc(-c7ccccc7)ccc6c45)c23)cc1]

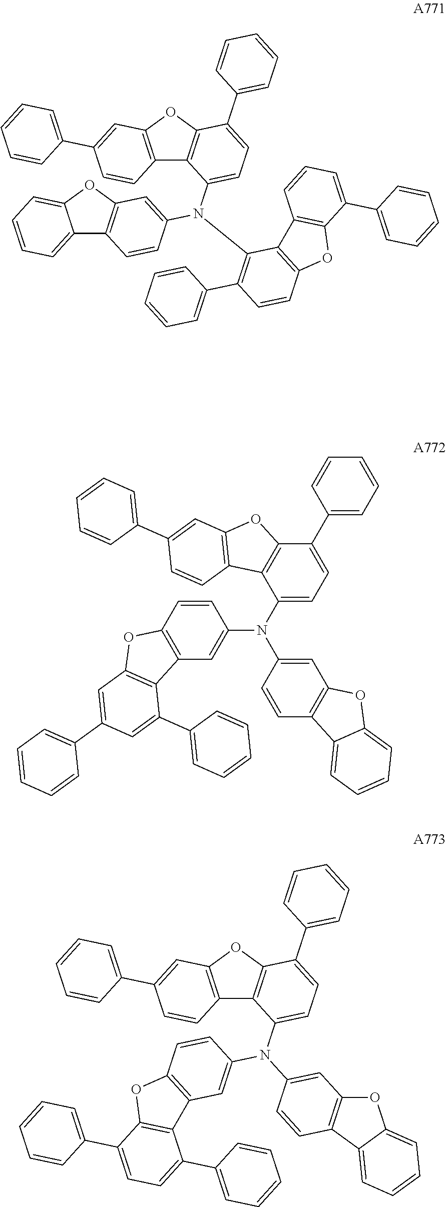 [SMILES: c1ccc(-c2ccc3c(c2)oc2c(-c4ccccc4)ccc(N(c4ccc5c(c4)oc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5c(c4)oc4ccccc45)c4ccc5oc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5oc6c(-c7ccccc7)ccc(-c7ccccc7)c6c5c4)c23)cc1]

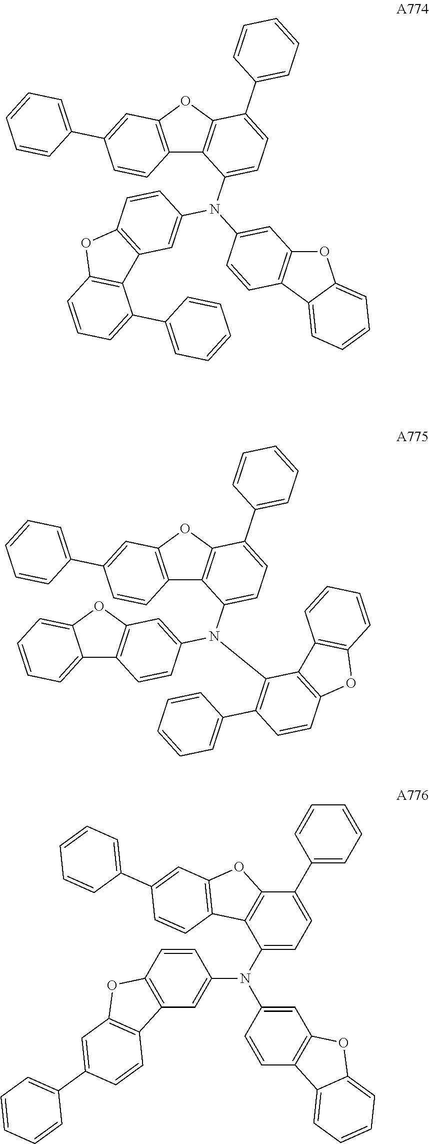 [SMILES: c1ccc(-c2ccc3c(c2)oc2c(-c4ccccc4)ccc(N(c4ccc5c(c4)oc4ccccc45)c4ccc5oc6cccc(-c7ccccc7)c6c5c4)c23)cc1 ; c1ccc(-c2ccc3c(c2)oc2c(-c4ccccc4)ccc(N(c4ccc5c(c4)oc4ccccc45)c4c(-c5ccccc5)ccc5oc6ccccc6c45)c23)cc1 ; c1ccc(-c2ccc3c(c2)oc2ccc(N(c4ccc5c(c4)oc4ccccc45)c4ccc(-c5ccccc5)c5oc6cc(-c7ccccc7)ccc6c45)cc23)cc1]

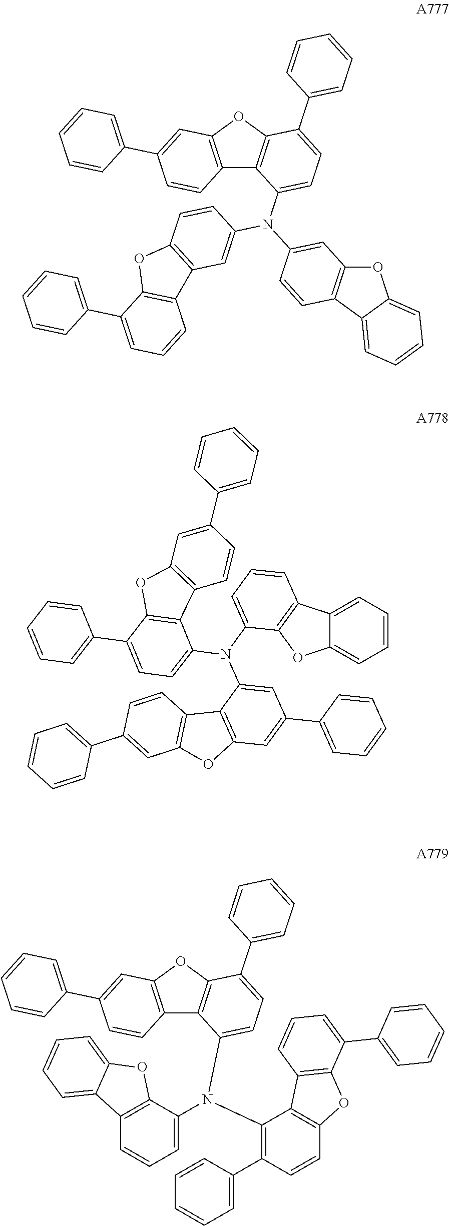 [SMILES: c1ccc(-c2ccc3c(c2)oc2c(-c4ccccc4)ccc(N(c4ccc5c(c4)oc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4oc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4cccc5c4oc4ccccc45)c4c(-c5ccccc5)ccc5oc6c(-c7ccccc7)cccc6c45)c23)cc1]

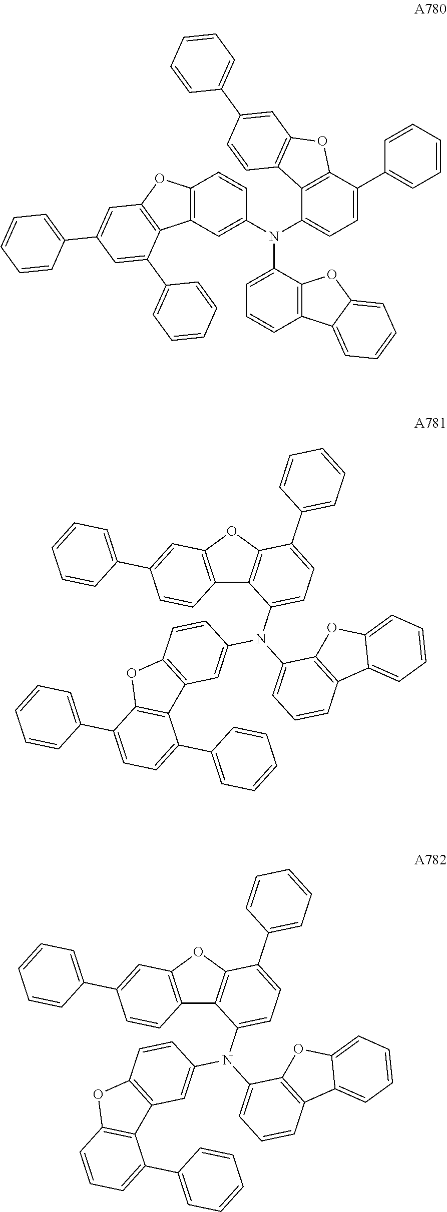 [SMILES: c1ccc(-c2ccc3c(c2)oc2c(-c4ccccc4)ccc(N(c4ccc5oc6cc(-c7ccccc7)cc(-c7ccccc7)c6c5c4)c4cccc5c4oc4ccccc45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5c4oc4ccccc45)c23)cc1 ; c1ccc(-c2ccc3c(c2)oc2c(-c4ccccc4)ccc(N(c4ccc5oc6cccc(-c7ccccc7)c6c5c4)c4cccc5c4oc4ccccc45)c23)cc1]

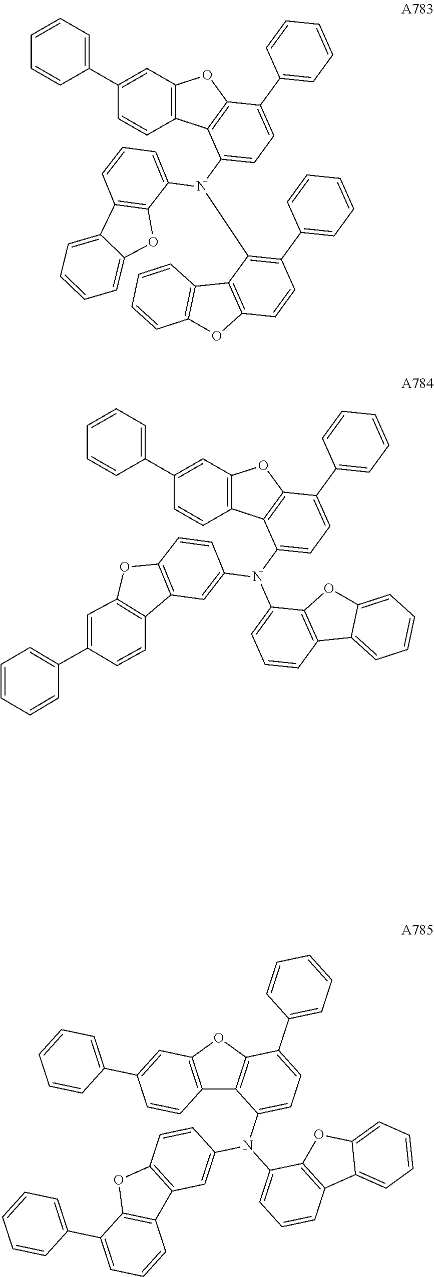 [SMILES: c1ccc(-c2ccc3c(c2)oc2c(-c4ccccc4)ccc(N(c4cccc5c4oc4ccccc45)c4c(-c5ccccc5)ccc5oc6ccccc6c45)c23)cc1 ; c1ccc(-c2ccc3c(c2)oc2ccc(N(c4cccc5c4oc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4cccc5c4oc4ccccc45)c23)cc1]

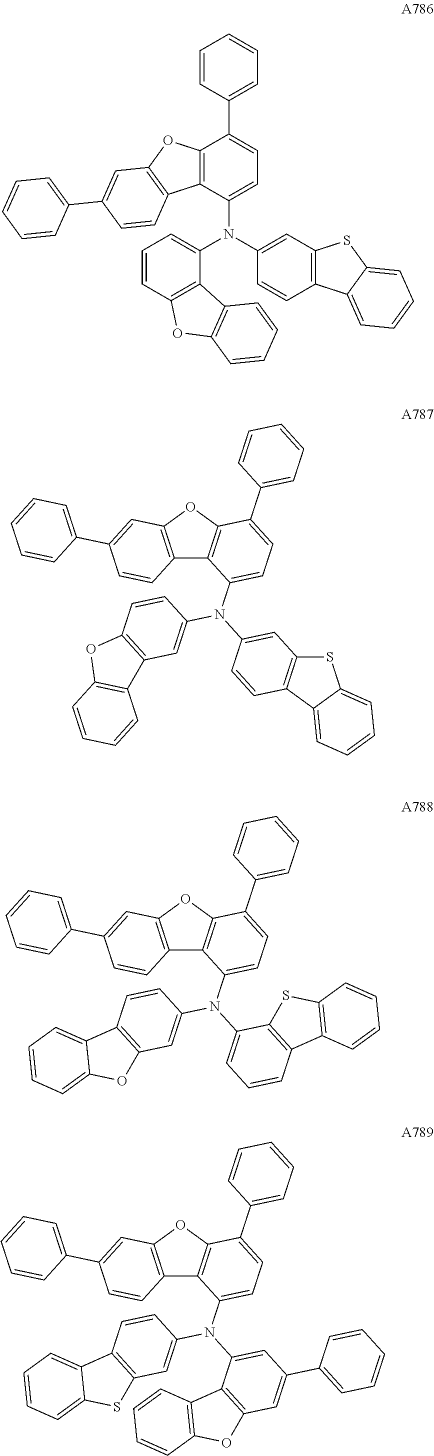 [SMILES: c1ccc(-c2ccc3c(c2)oc2c(-c4ccccc4)ccc(N(c4ccc5c(c4)sc4ccccc45)c4cccc5oc6ccccc6c45)c23)cc1 ; c1ccc(-c2ccc3c(c2)oc2c(-c4ccccc4)ccc(N(c4ccc5c(c4)sc4ccccc45)c4ccc5oc6ccccc6c5c4)c23)cc1 ; c1ccc(-c2ccc3c(c2)oc2c(-c4ccccc4)ccc(N(c4ccc5c(c4)oc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5oc6ccccc6c45)c23)cc1]

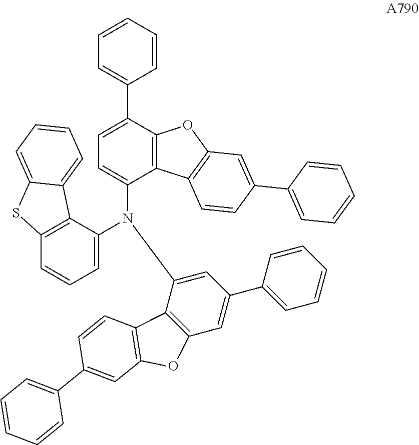 [SMILES: c1ccc(-c2ccc3c(c2)oc2cc(-c4ccccc4)cc(N(c4cccc5sc6ccccc6c45)c4ccc(-c5ccccc5)c5oc6cc(-c7ccccc7)ccc6c45)c23)cc1]

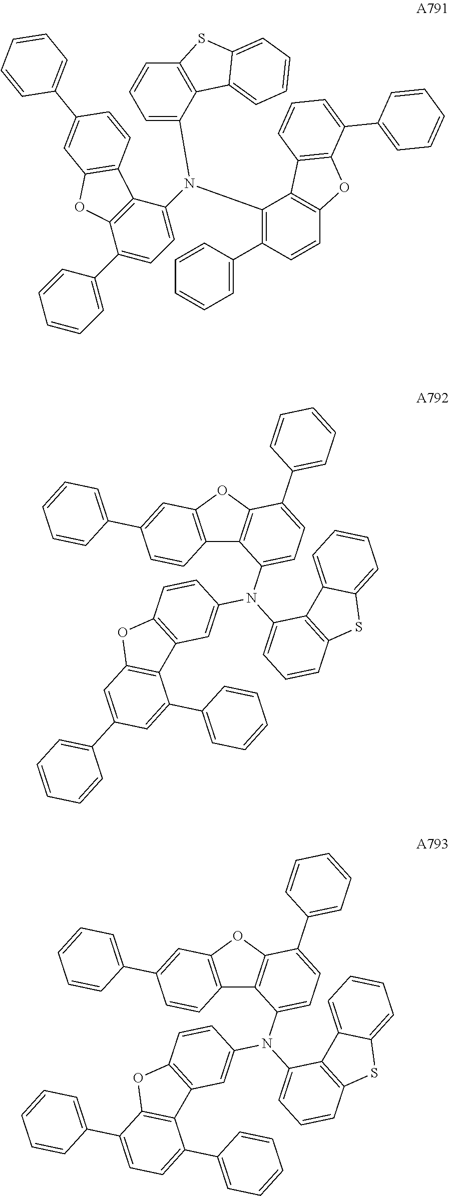 [SMILES: c1ccc(-c2ccc3c(c2)oc2c(-c4ccccc4)ccc(N(c4c(-c5ccccc5)ccc5oc6c(-c7ccccc7)cccc6c45)c4cccc5sc6ccccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5sc6ccccc6c45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5sc6ccccc6c45)c23)cc1]

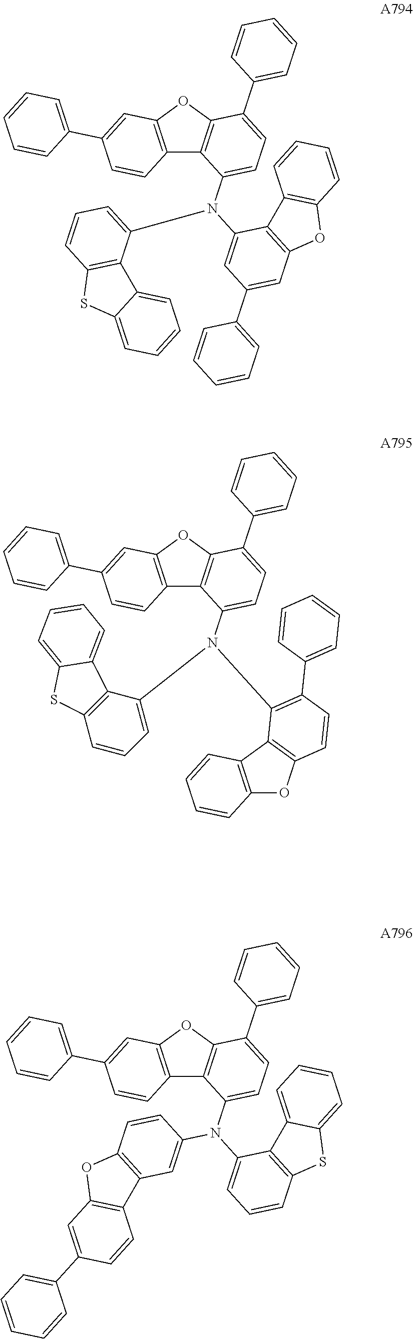 [SMILES: c1ccc(-c2ccc3c(c2)oc2c(-c4ccccc4)ccc(N(c4cc(-c5ccccc5)cc5oc6ccccc6c45)c4cccc5sc6ccccc6c45)c23)cc1 ; c1ccc(-c2ccc3c(c2)oc2c(-c4ccccc4)ccc(N(c4c(-c5ccccc5)ccc5oc6ccccc6c45)c4cccc5sc6ccccc6c45)c23)cc1 ; c1ccc(-c2ccc3c(c2)oc2ccc(N(c4cccc5sc6ccccc6c45)c4ccc(-c5ccccc5)c5oc6cc(-c7ccccc7)ccc6c45)cc23)cc1]

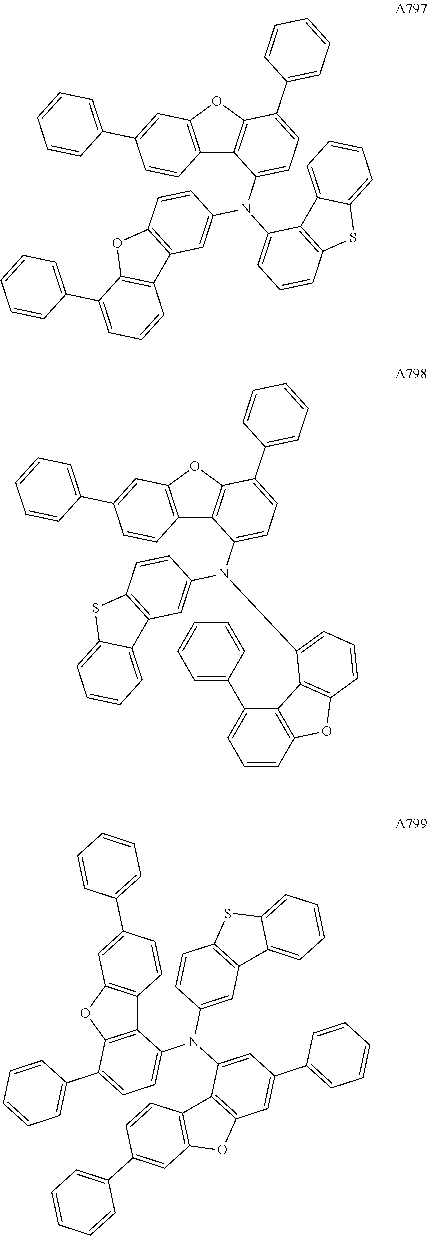 [SMILES: c1ccc(-c2ccc3c(c2)oc2c(-c4ccccc4)ccc(N(c4ccc5oc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5sc6ccccc6c5c4)c4ccc(-c5ccccc5)c5oc6cc(-c7ccccc7)ccc6c45)c23)cc1]

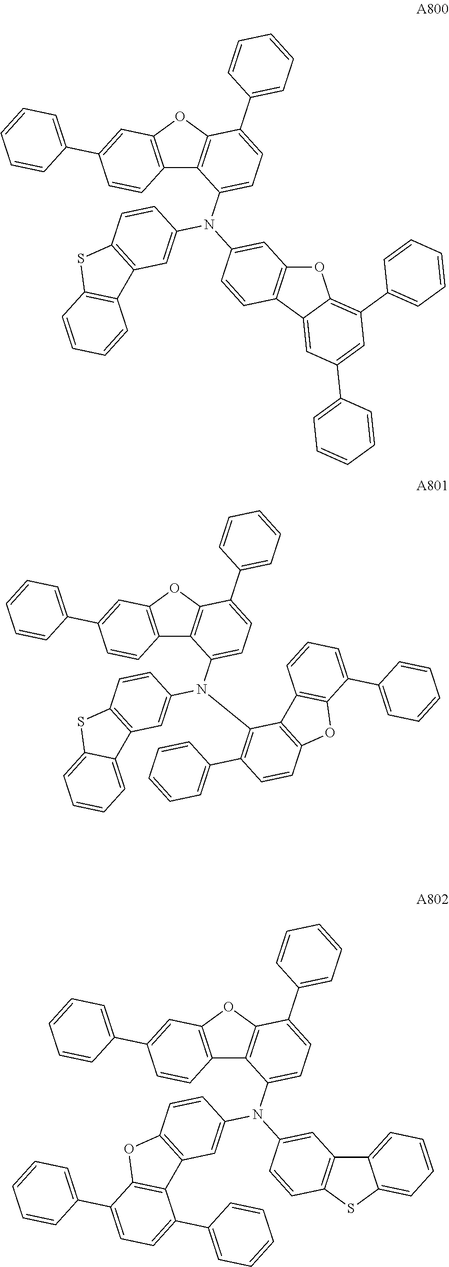 [SMILES: c1ccc(-c2ccc3c(c2)oc2c(-c4ccccc4)ccc(N(c4ccc5c(c4)oc4c(-c6ccccc6)cc(-c6ccccc6)cc45)c4ccc5sc6ccccc6c5c4)c23)cc1 ; c1ccc(-c2ccc3c(c2)oc2c(-c4ccccc4)ccc(N(c4ccc5sc6ccccc6c5c4)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5sc6ccccc6c5c4)c4ccc5oc6c(-c7ccccc7)ccc(-c7ccccc7)c6c5c4)c23)cc1]

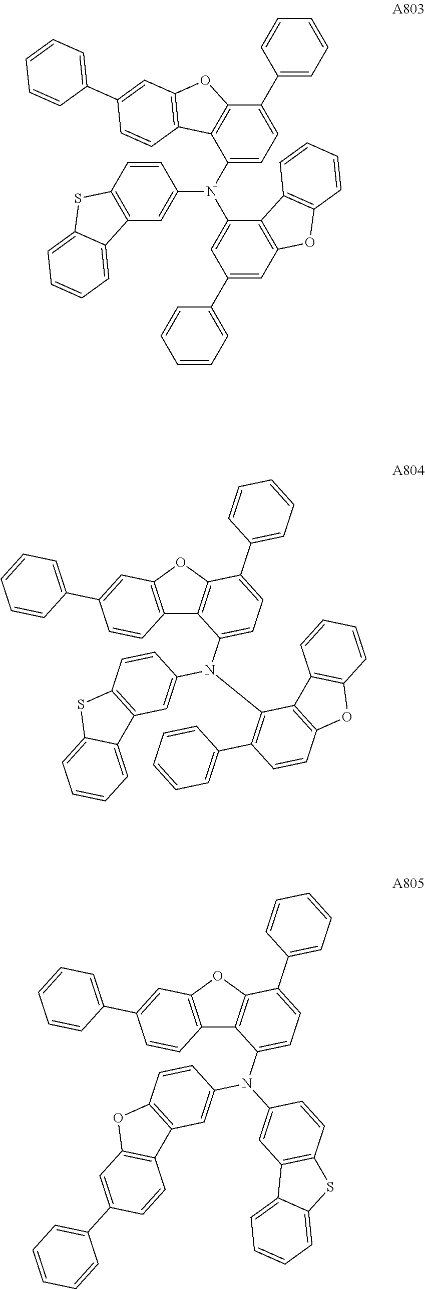 [SMILES: c1ccc(-c2ccc3c(c2)oc2c(-c4ccccc4)ccc(N(c4ccc5sc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc5sc6ccccc6c5c4)c4c(-c5ccccc5)ccc5oc6ccccc6c45)c23)cc1 ; c1ccc(-c2ccc3c(c2)oc2ccc(N(c4ccc5sc6ccccc6c5c4)c4ccc(-c5ccccc5)c5oc6cc(-c7ccccc7)ccc6c45)cc23)cc1]

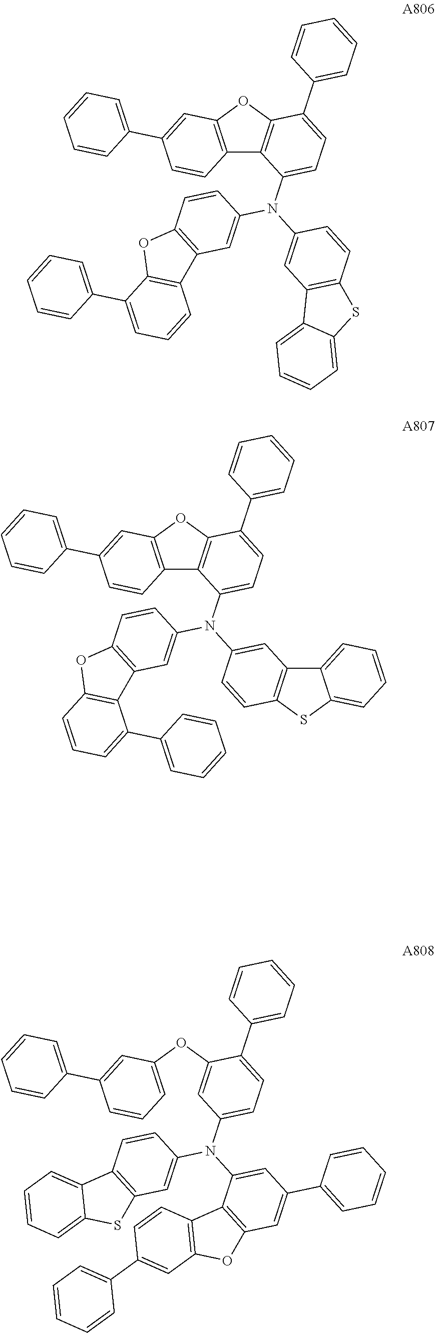 [SMILES: c1ccc(-c2ccc3c(c2)oc2c(-c4ccccc4)ccc(N(c4ccc5oc6c(-c7ccccc7)cccc6c5c4)c4ccc5sc6ccccc6c5c4)c23)cc1 ; c1ccc(-c2ccc3c(c2)oc2c(-c4ccccc4)ccc(N(c4ccc5sc6ccccc6c5c4)c4ccc5oc6cccc(-c7ccccc7)c6c5c4)c23)cc1 ; c1ccc(-c2cccc(Oc3cc(N(c4ccc5c(c4)sc4ccccc45)c4cc(-c5ccccc5)cc5oc6cc(-c7ccccc7)ccc6c45)ccc3-c3ccccc3)c2)cc1]

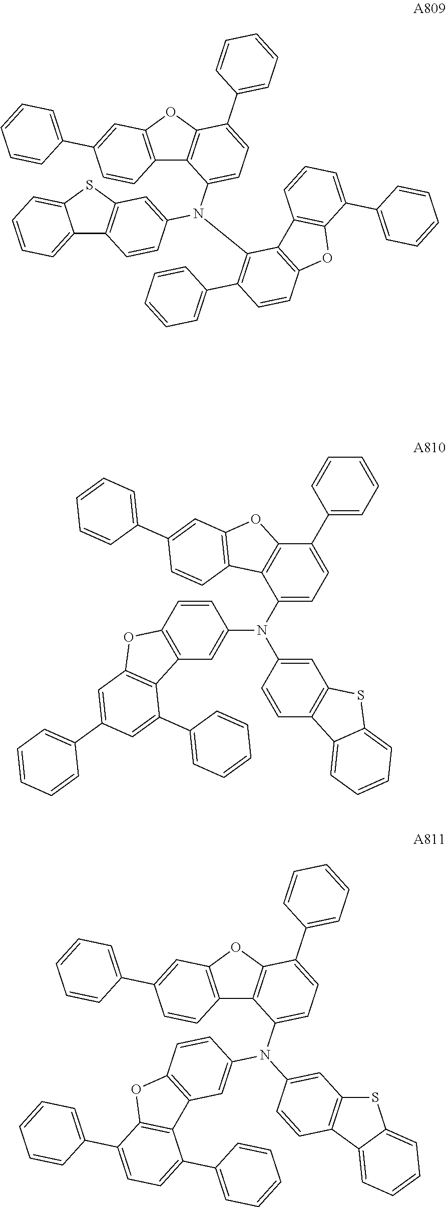 [SMILES: c1ccc(-c2ccc3c(c2)oc2c(-c4ccccc4)ccc(N(c4ccc5c(c4)sc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5oc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5oc6c(-c7ccccc7)ccc(-c7ccccc7)c6c5c4)c23)cc1]

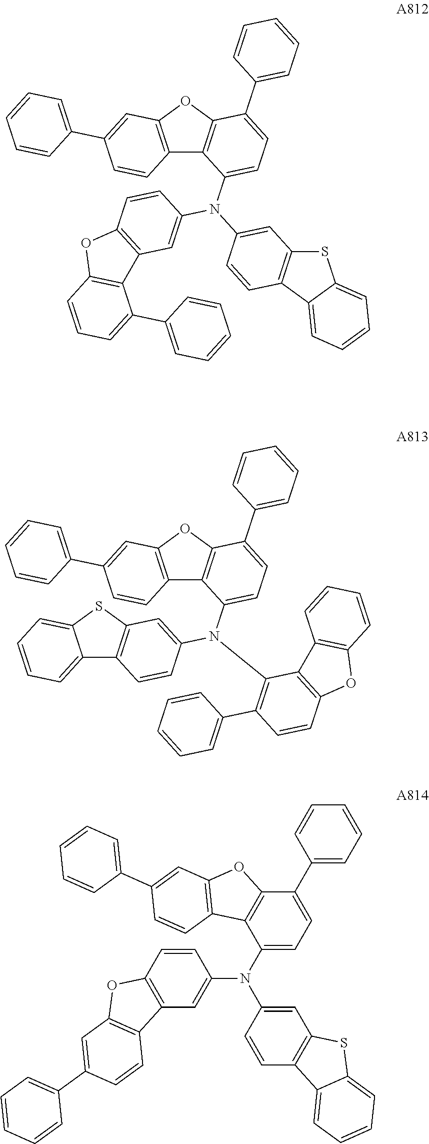 [SMILES: c1ccc(-c2ccc3c(c2)oc2c(-c4ccccc4)ccc(N(c4ccc5c(c4)sc4ccccc45)c4ccc5oc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5oc6ccccc6c45)c23)cc1 ; c1ccc(-c2ccc3c(c2)oc2ccc(N(c4ccc5c(c4)sc4ccccc45)c4ccc(-c5ccccc5)c5oc6cc(-c7ccccc7)ccc6c45)cc23)cc1]

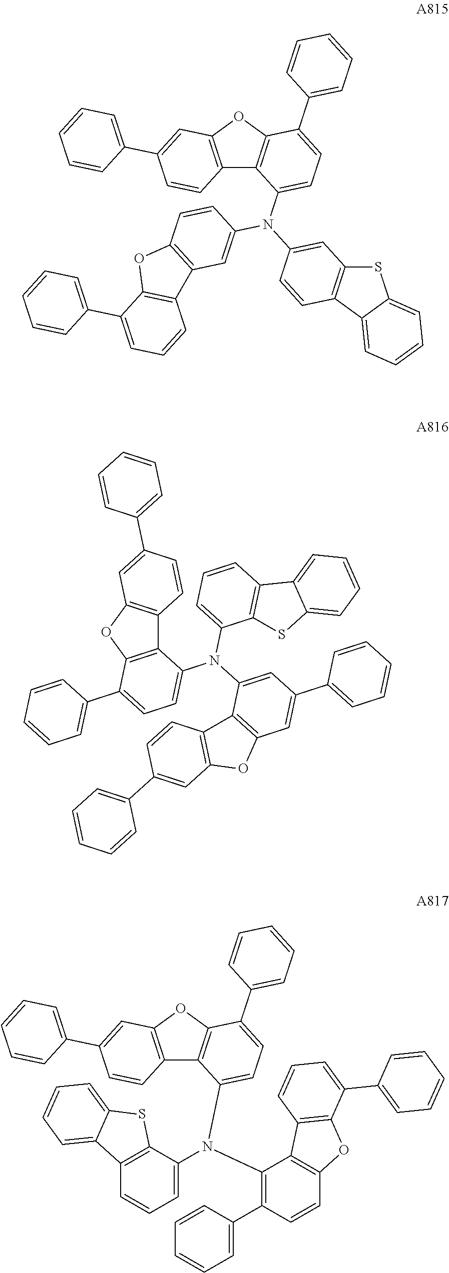 [SMILES: c1ccc(-c2ccc3c(c2)oc2c(-c4ccccc4)ccc(N(c4ccc5c(c4)sc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4sc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4cccc5c4sc4ccccc45)c4c(-c5ccccc5)ccc5oc6c(-c7ccccc7)cccc6c45)c23)cc1]

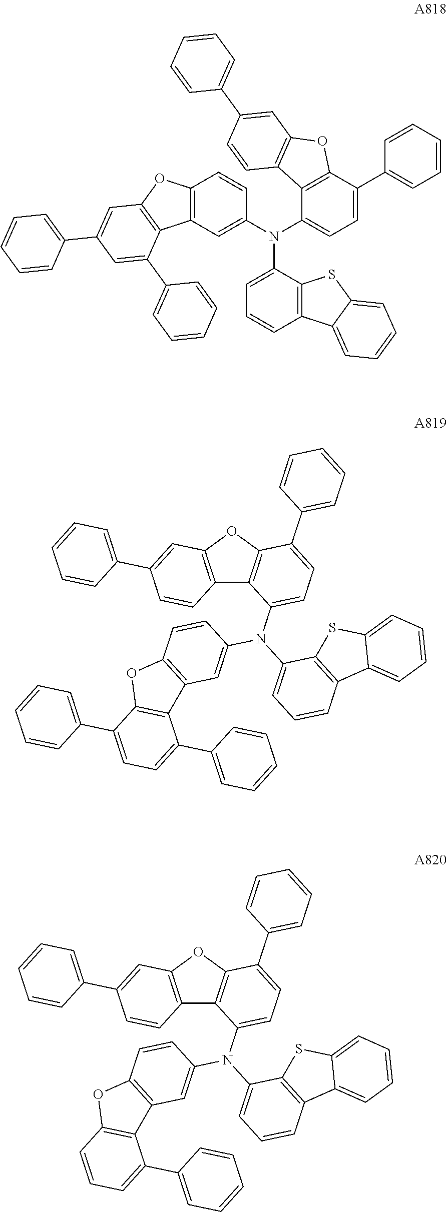 [SMILES: c1ccc(-c2ccc3c(c2)oc2c(-c4ccccc4)ccc(N(c4ccc5oc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5c4sc4ccccc45)c23)cc1 ; c1ccc(-c2ccc3c(c2)oc2c(-c4ccccc4)ccc(N(c4ccc5oc6cccc(-c7ccccc7)c6c5c4)c4cccc5c4sc4ccccc45)c23)cc1]

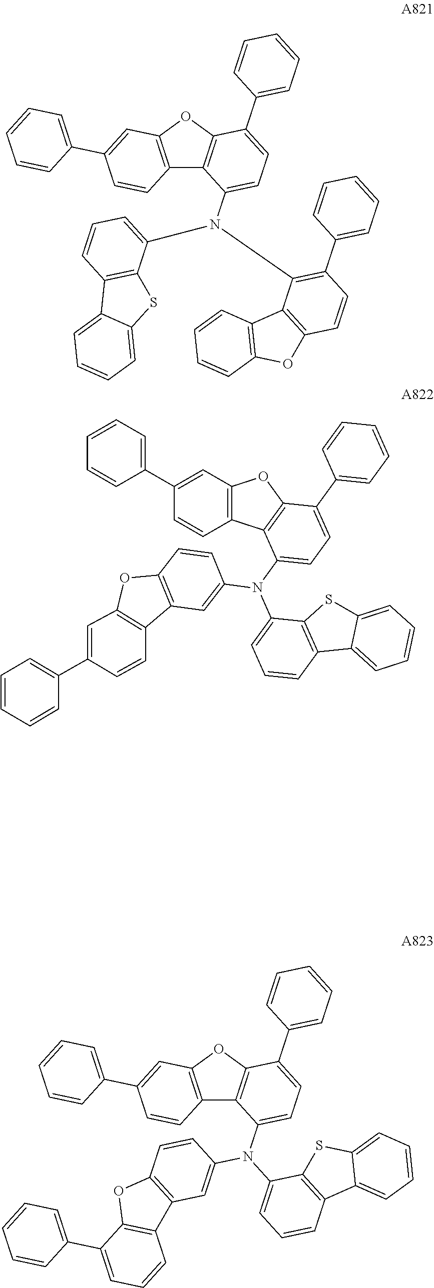

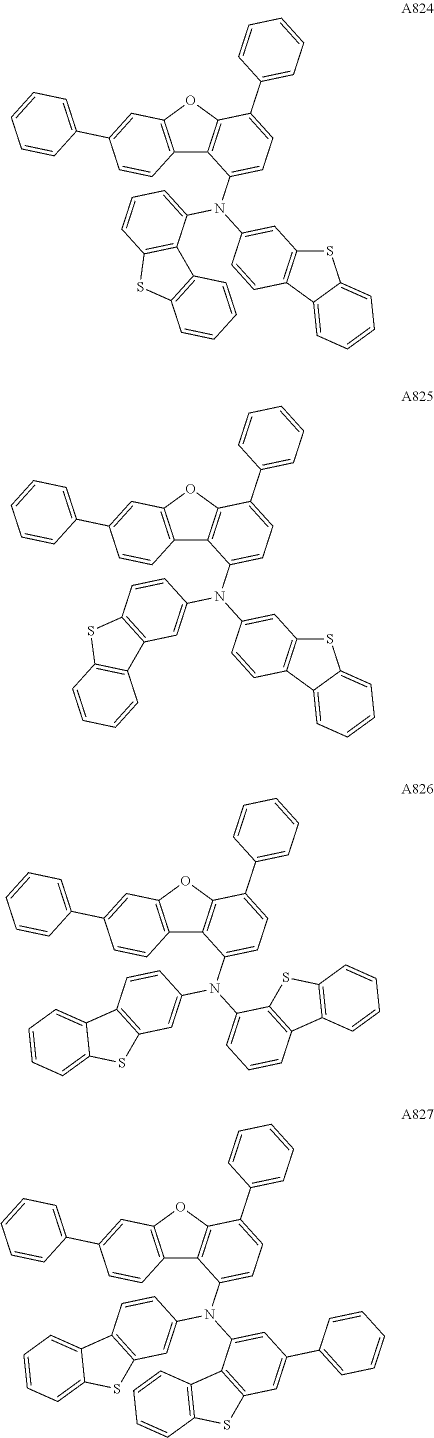 [SMILES: c1ccc(-c2ccc3c(c2)oc2c(-c4ccccc4)ccc(N(c4ccc5c(c4)sc4ccccc45)c4cccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6ccccc6c5c4)c23)cc1 ; c1ccc(-c2ccc3c(c2)oc2c(-c4ccccc4)ccc(N(c4ccc5c(c4)sc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5sc6ccccc6c45)c23)cc1]

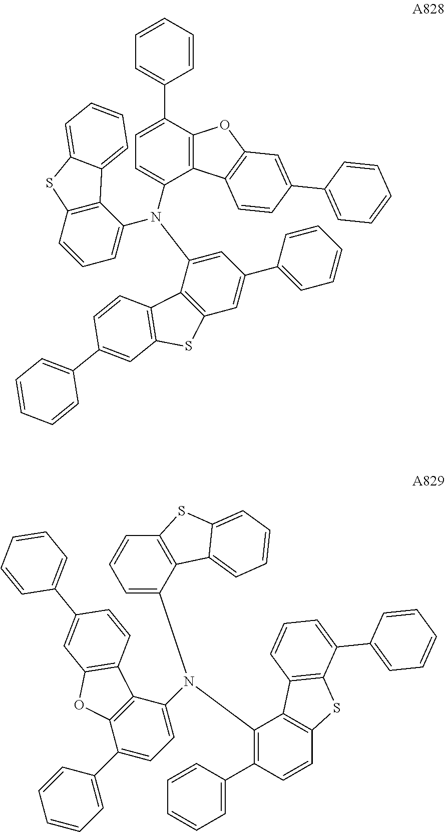 [SMILES: c1ccc(-c2ccc3c(c2)oc2c(-c4ccccc4)ccc(N(c4cccc5sc6ccccc6c45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5sc6ccccc6c45)c4c(-c5ccccc5)ccc5sc6c(-c7ccccc7)cccc6c45)c23)cc1]

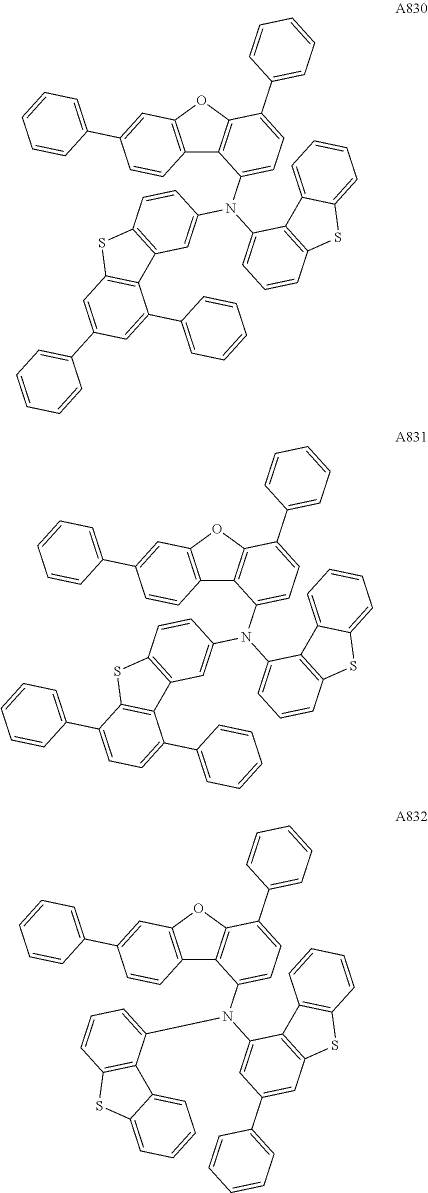 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5sc6ccccc6c45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5sc6ccccc6c45)c23)cc1 ; c1ccc(-c2ccc3c(c2)oc2c(-c4ccccc4)ccc(N(c4cccc5sc6ccccc6c45)c4cc(-c5ccccc5)cc5sc6ccccc6c45)c23)cc1]

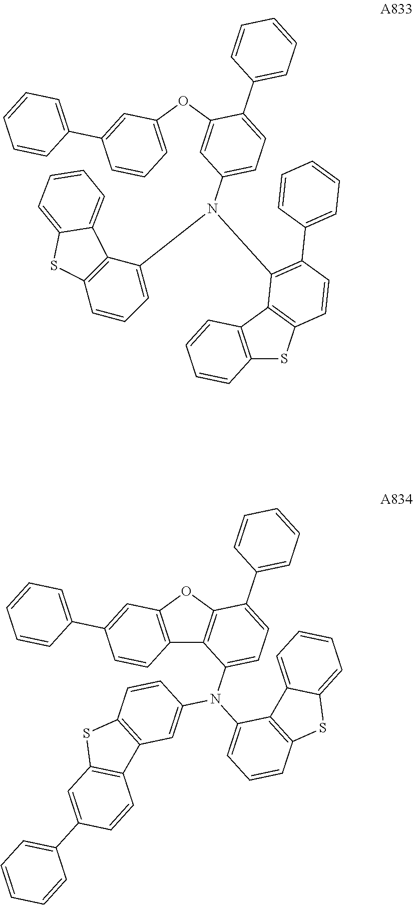 [SMILES: c1ccc(-c2cccc(Oc3cc(N(c4cccc5sc6ccccc6c45)c4c(-c5ccccc5)ccc5sc6ccccc6c45)ccc3-c3ccccc3)c2)cc1 ; c1ccc(-c2ccc3c(c2)oc2c(-c4ccccc4)ccc(N(c4ccc5sc6cc(-c7ccccc7)ccc6c5c4)c4cccc5sc6ccccc6c45)c23)cc1]

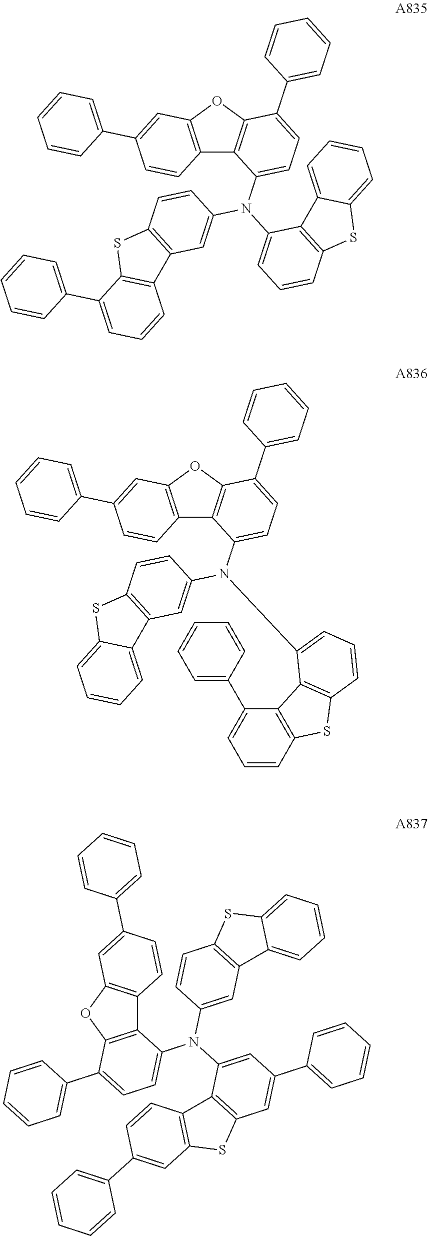 [SMILES: c1ccc(-c2ccc3c(c2)oc2c(-c4ccccc4)ccc(N(c4ccc5sc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5sc6cccc(-c7ccccc7)c6c45)c23)cc1 ; c1ccc(-c2ccc3c(c2)oc2c(-c4ccccc4)ccc(N(c4ccc5sc6ccccc6c5c4)c4cc(-c5ccccc5)cc5sc6cc(-c7ccccc7)ccc6c45)c23)cc1]

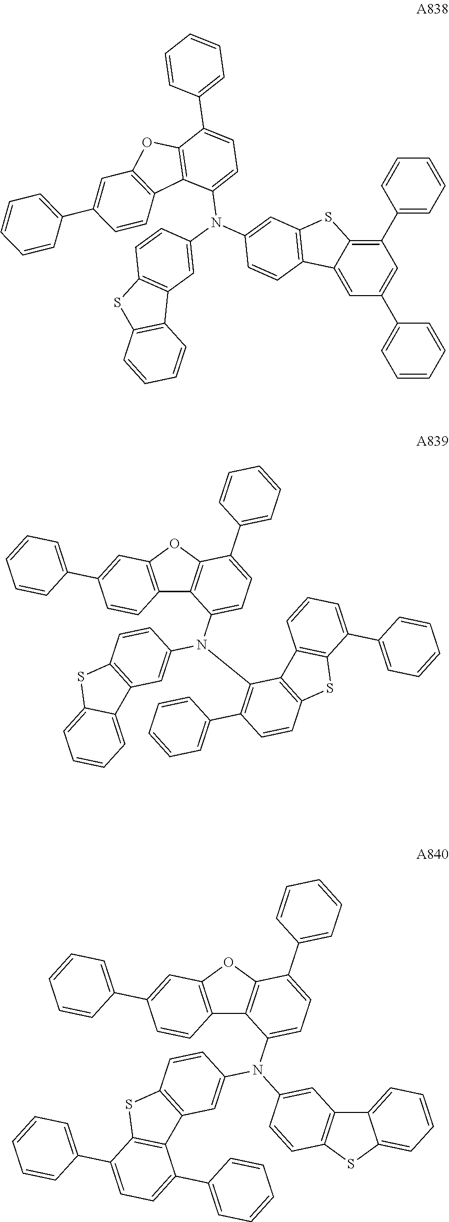 [SMILES: c1ccc(-c2ccc3c(c2)oc2c(-c4ccccc4)ccc(N(c4ccc5c(c4)sc4c(-c6ccccc6)cc(-c6ccccc6)cc45)c4ccc5sc6ccccc6c5c4)c23)cc1 ; c1ccc(-c2ccc3c(c2)oc2c(-c4ccccc4)ccc(N(c4ccc5sc6ccccc6c5c4)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6ccccc6c5c4)c4ccc5sc6c(-c7ccccc7)ccc(-c7ccccc7)c6c5c4)c23)cc1]

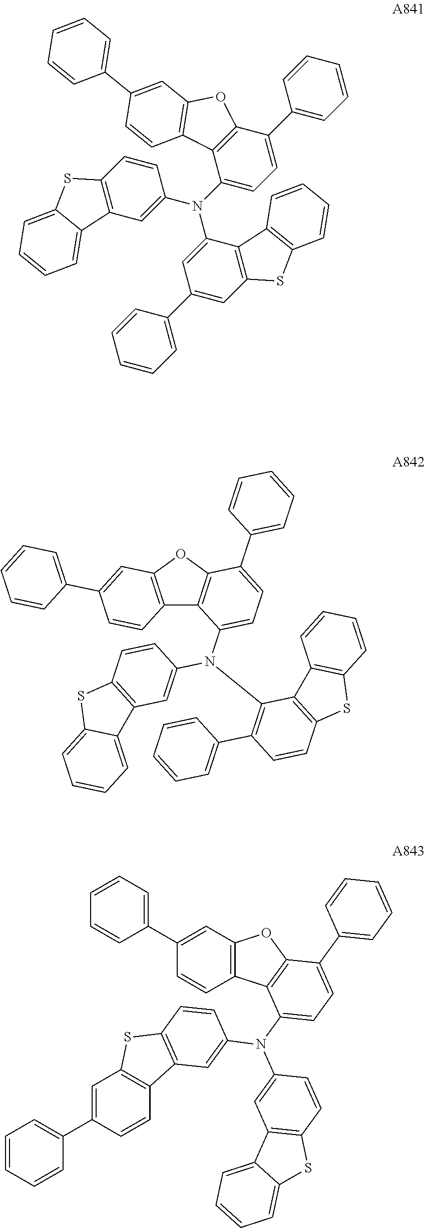 [SMILES: c1ccc(-c2ccc3c(c2)oc2c(-c4ccccc4)ccc(N(c4ccc5sc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc5sc6ccccc6c5c4)c4c(-c5ccccc5)ccc5sc6ccccc6c45)c23)cc1 ; c1ccc(-c2ccc3c(c2)oc2c(-c4ccccc4)ccc(N(c4ccc5sc6ccccc6c5c4)c4ccc5sc6cc(-c7ccccc7)ccc6c5c4)c23)cc1]

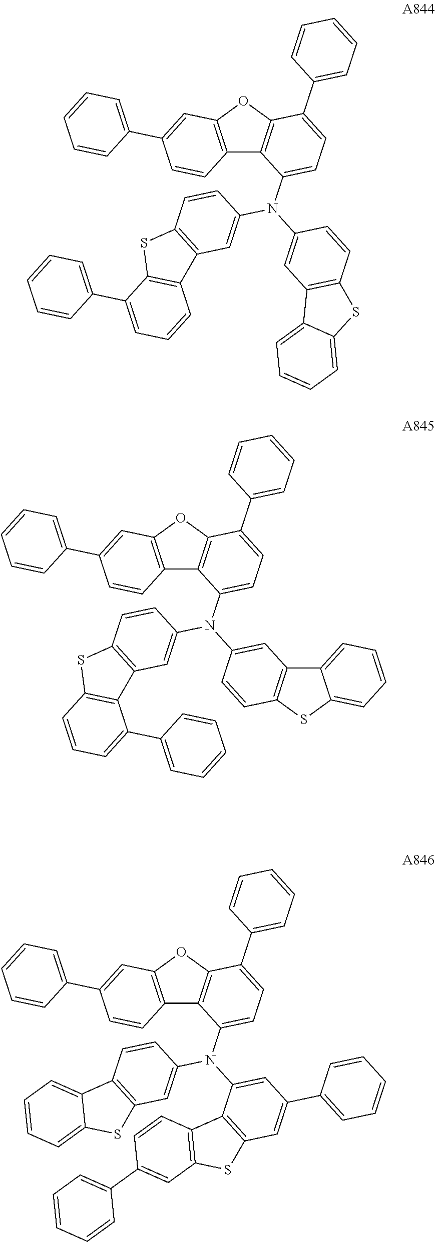 [SMILES: c1ccc(-c2ccc3c(c2)oc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)oc2c(-c4ccccc4)ccc(N(c4ccc5sc6ccccc6c5c4)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4cc(-c5ccccc5)cc5sc6cc(-c7ccccc7)ccc6c45)c23)cc1]

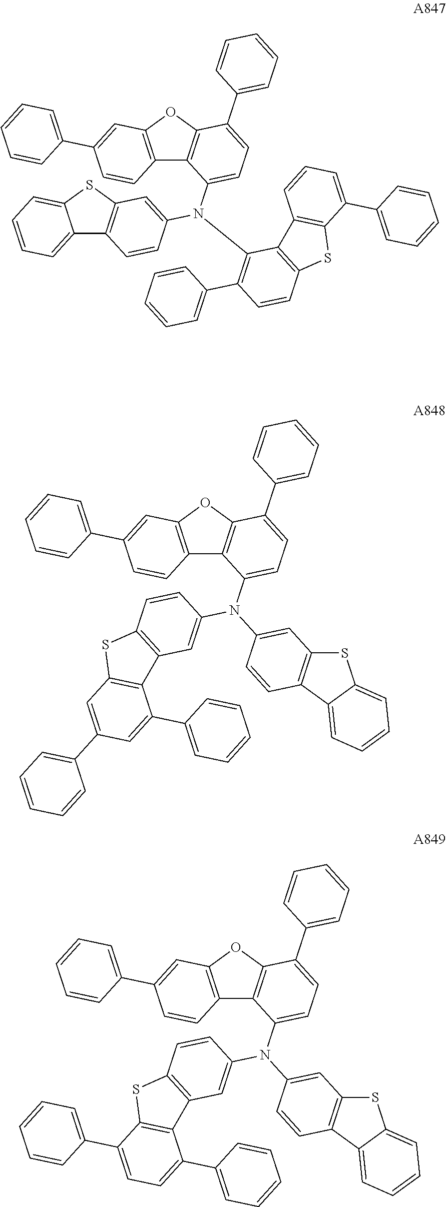 [SMILES: c1ccc(-c2ccc3c(c2)oc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5sc6c(-c7ccccc7)ccc(-c7ccccc7)c6c5c4)c23)cc1]

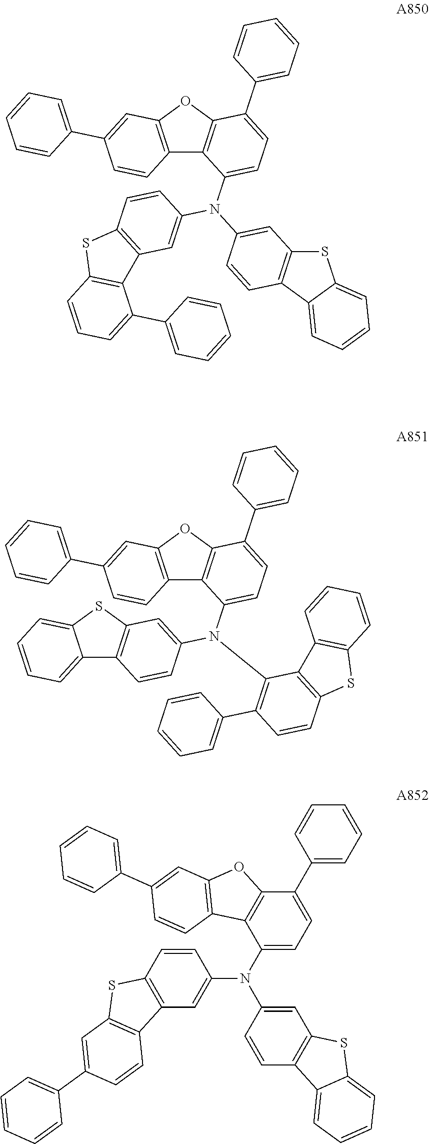 [SMILES: c1ccc(-c2ccc3c(c2)oc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)ccc6c5c4)c23)cc1]

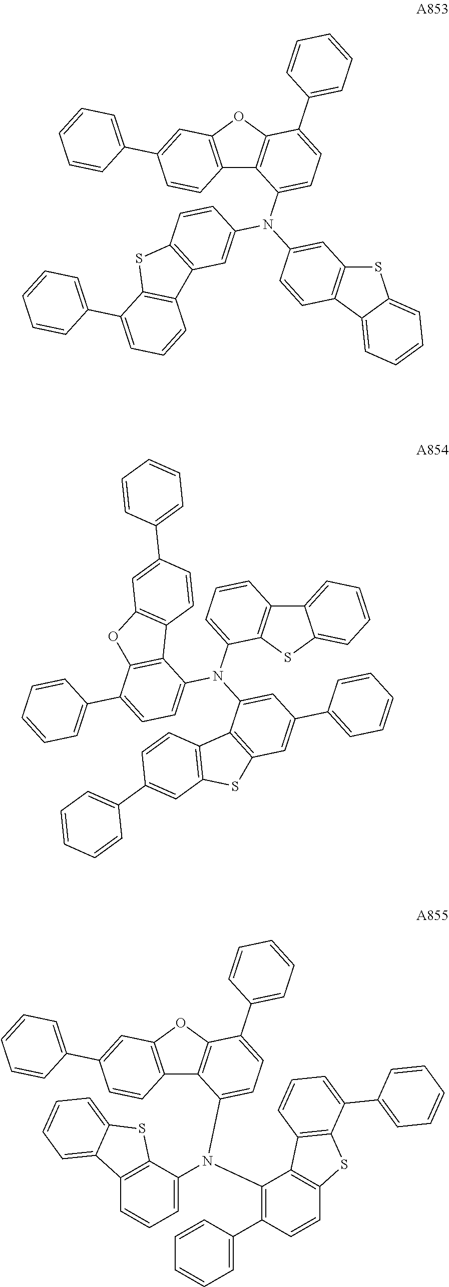 [SMILES: c1ccc(-c2ccc3c(c2)oc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)oc2c(-c4ccccc4)ccc(N(c4cccc5c4sc4ccccc45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5c4sc4ccccc45)c4c(-c5ccccc5)ccc5sc6c(-c7ccccc7)cccc6c45)c23)cc1]

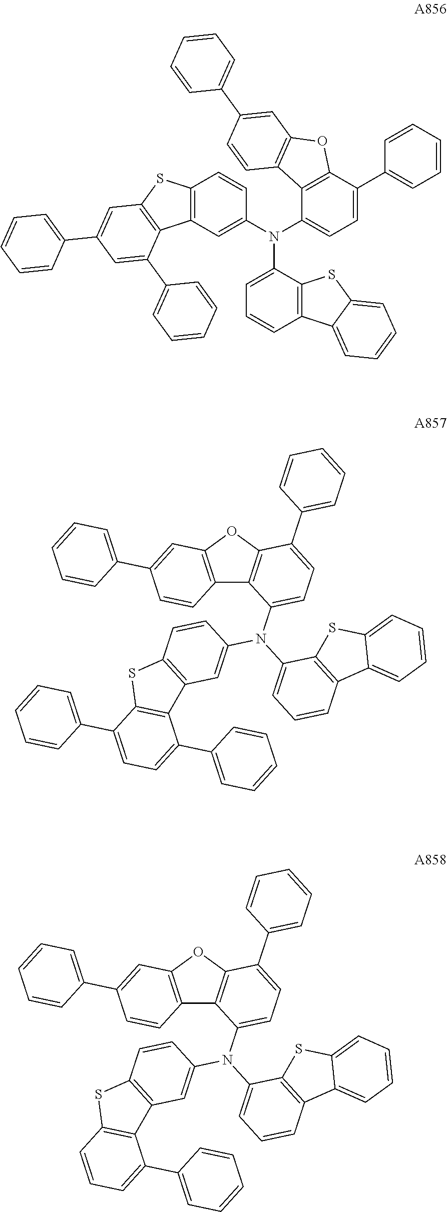 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6cccc(-c7ccccc7)c6c5c4)c4cccc5c4sc4ccccc45)c23)cc1]

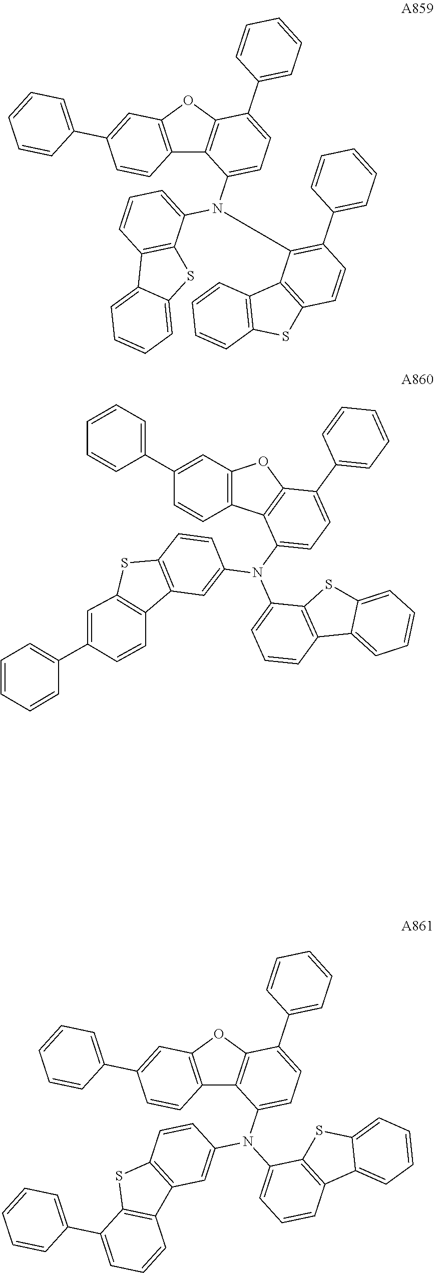 [SMILES: c1ccc(-c2ccc3c(c2)oc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5sc6cc(-c7ccccc7)ccc6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)cccc6c5c4)c4cccc5c4sc4ccccc45)c23)cc1]

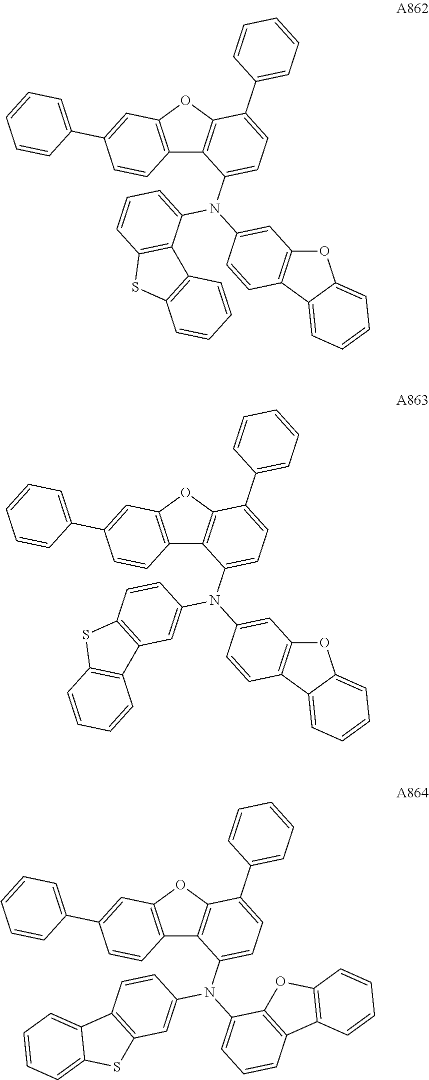 [SMILES: c1ccc(-c2ccc3c(c2)oc2c(-c4ccccc4)ccc(N(c4ccc5c(c4)oc4ccccc45)c4cccc5sc6ccccc6c45)c23)cc1 ; c1ccc(-c2ccc3c(c2)oc2c(-c4ccccc4)ccc(N(c4ccc5c(c4)oc4ccccc45)c4ccc5sc6ccccc6c5c4)c23)cc1 ; c1ccc(-c2ccc3c(c2)oc2c(-c4ccccc4)ccc(N(c4ccc5c(c4)sc4ccccc45)c4cccc5c4oc4ccccc45)c23)cc1]

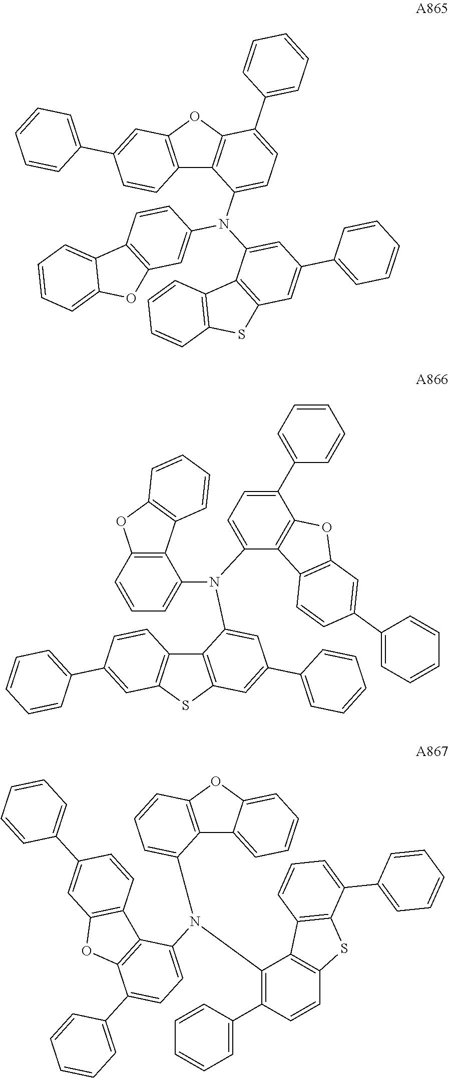 [SMILES: c1ccc(-c2ccc3c(c2)oc2c(-c4ccccc4)ccc(N(c4ccc5c(c4)oc4ccccc45)c4cc(-c5ccccc5)cc5sc6ccccc6c45)c23)cc1 ; c1ccc(-c2ccc3c(c2)oc2c(-c4ccccc4)ccc(N(c4cccc5oc6ccccc6c45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5oc6ccccc6c45)c4c(-c5ccccc5)ccc5sc6c(-c7ccccc7)cccc6c45)c23)cc1]

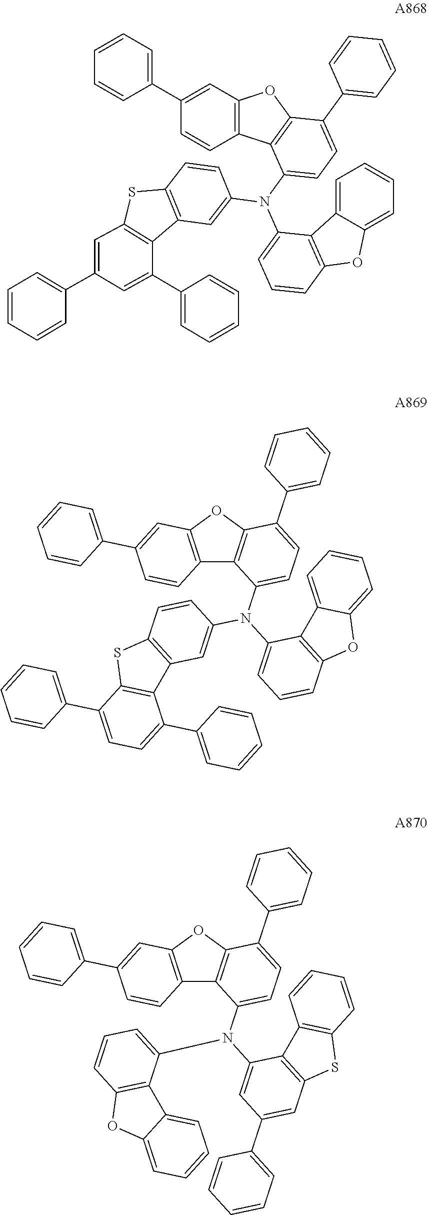 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5oc6ccccc6c45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5oc6ccccc6c45)c23)cc1 ; c1ccc(-c2ccc3c(c2)oc2c(-c4ccccc4)ccc(N(c4cccc5oc6ccccc6c45)c4cc(-c5ccccc5)cc5sc6ccccc6c45)c23)cc1]

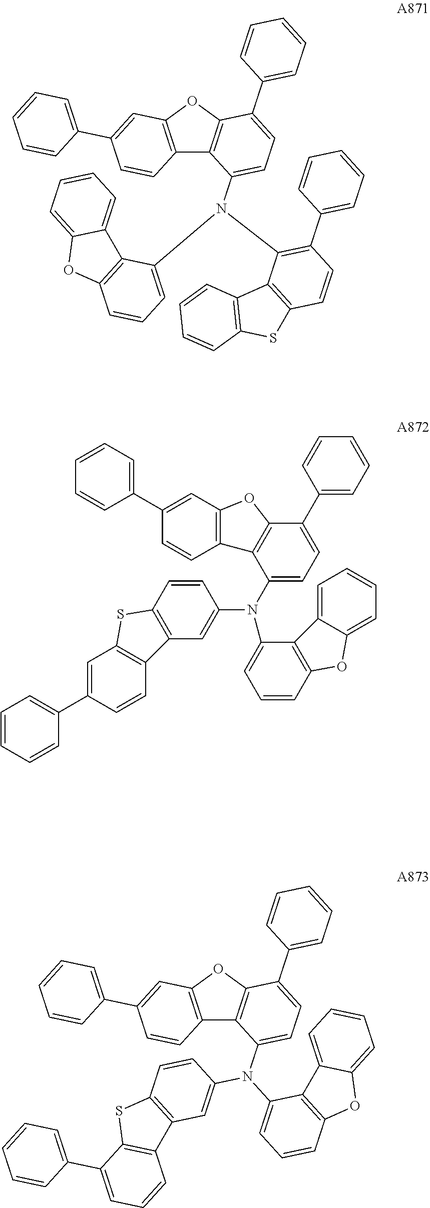 [SMILES: c1ccc(-c2ccc3c(c2)oc2c(-c4ccccc4)ccc(N(c4cccc5oc6ccccc6c45)c4c(-c5ccccc5)ccc5sc6ccccc6c45)c23)cc1 ; c1ccc(-c2ccc3c(c2)oc2c(-c4ccccc4)ccc(N(c4ccc5sc6cc(-c7ccccc7)ccc6c5c4)c4cccc5oc6ccccc6c45)c23)cc1 ; c1ccc(-c2ccc3c(c2)oc2c(-c4ccccc4)ccc(N(c4ccc5sc6c(-c7ccccc7)cccc6c5c4)c4cccc5oc6ccccc6c45)c23)cc1]

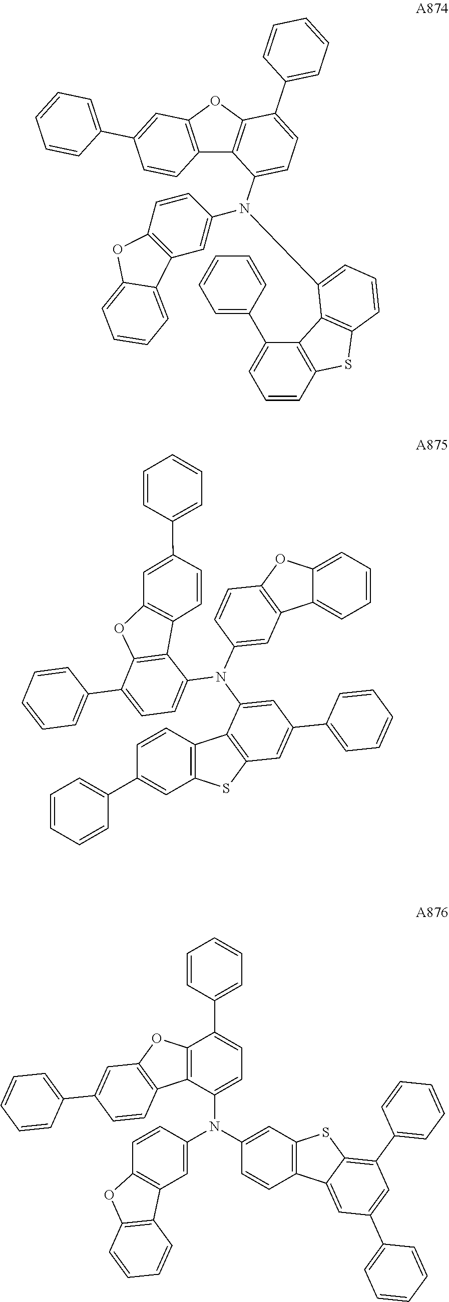 [SMILES: c1ccc(-c2ccc3c(c2)oc2c(-c4ccccc4)ccc(N(c4ccc5oc6ccccc6c5c4)c4cccc5sc6cccc(-c7ccccc7)c6c45)c23)cc1 ; c1ccc(-c2ccc3c(c2)oc2c(-c4ccccc4)ccc(N(c4ccc5oc6ccccc6c5c4)c4cc(-c5ccccc5)cc5sc6cc(-c7ccccc7)ccc6c45)c23)cc1 ; c1ccc(-c2ccc3c(c2)oc2c(-c4ccccc4)ccc(N(c4ccc5c(c4)sc4c(-c6ccccc6)cc(-c6ccccc6)cc45)c4ccc5oc6ccccc6c5c4)c23)cc1]

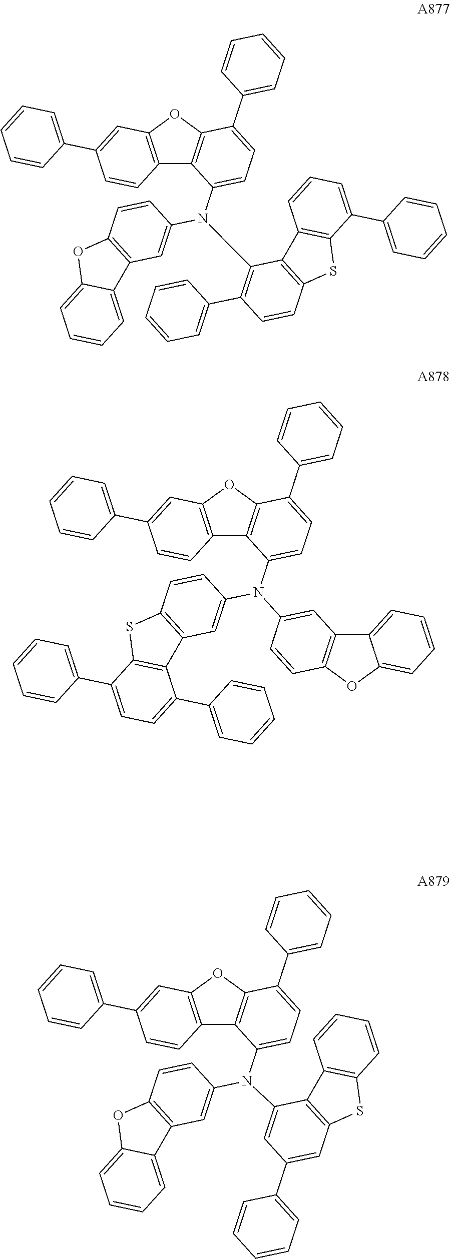 [SMILES: c1ccc(-c2ccc3c(c2)oc2c(-c4ccccc4)ccc(N(c4ccc5oc6ccccc6c5c4)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5oc6ccccc6c5c4)c4ccc5sc6c(-c7ccccc7)ccc(-c7ccccc7)c6c5c4)c23)cc1 ; c1ccc(-c2ccc3c(c2)oc2c(-c4ccccc4)ccc(N(c4ccc5oc6ccccc6c5c4)c4cc(-c5ccccc5)cc5sc6ccccc6c45)c23)cc1]

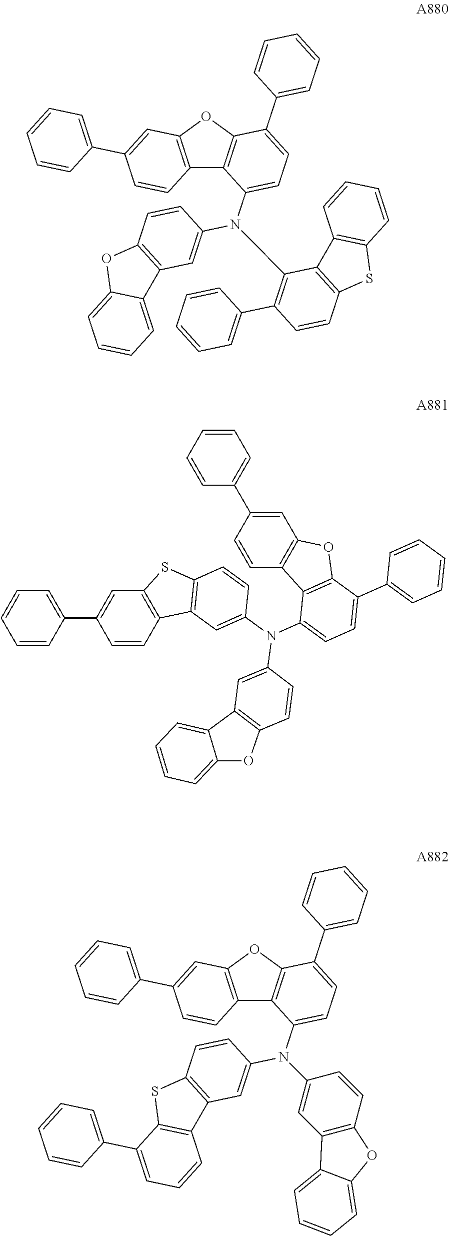 [SMILES: c1ccc(-c2ccc3c(c2)oc2c(-c4ccccc4)ccc(N(c4ccc5oc6ccccc6c5c4)c4c(-c5ccccc5)ccc5sc6ccccc6c45)c23)cc1 ; c1ccc(-c2ccc3c(c2)oc2c(-c4ccccc4)ccc(N(c4ccc5oc6ccccc6c5c4)c4ccc5sc6cc(-c7ccccc7)ccc6c5c4)c23)cc1 ; c1ccc(-c2ccc3c(c2)oc2c(-c4ccccc4)ccc(N(c4ccc5oc6ccccc6c5c4)c4ccc5sc6c(-c7ccccc7)cccc6c5c4)c23)cc1]

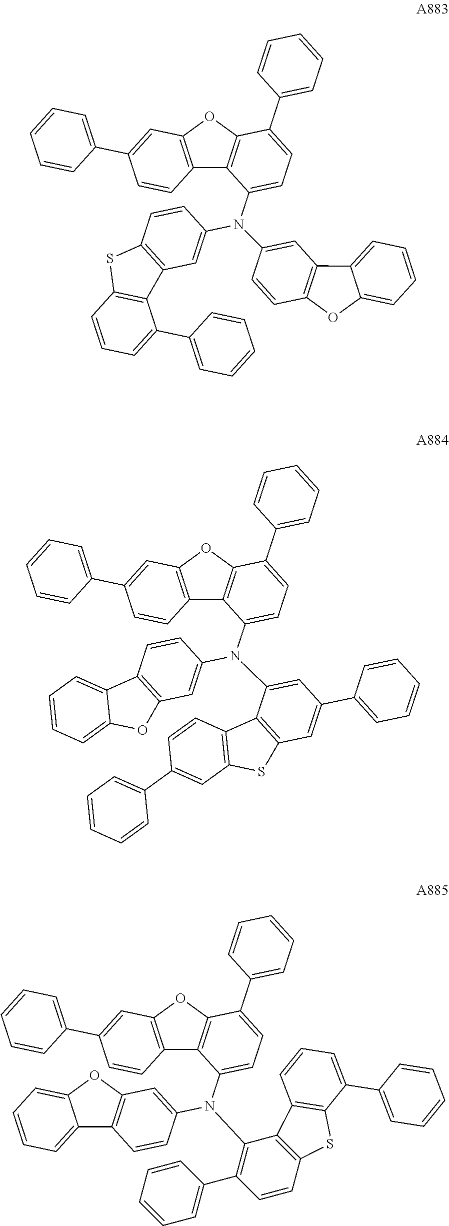 [SMILES: c1ccc(-c2ccc3c(c2)oc2c(-c4ccccc4)ccc(N(c4ccc5oc6ccccc6c5c4)c4ccc5sc6cccc(-c7ccccc7)c6c5c4)c23)cc1 ; c1ccc(-c2ccc3c(c2)oc2c(-c4ccccc4)ccc(N(c4ccc5c(c4)oc4ccccc45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4ccc5c(c4)oc4ccccc45)c4c(-c5ccccc5)ccc5sc6c(-c7ccccc7)cccc6c45)c23)cc1]

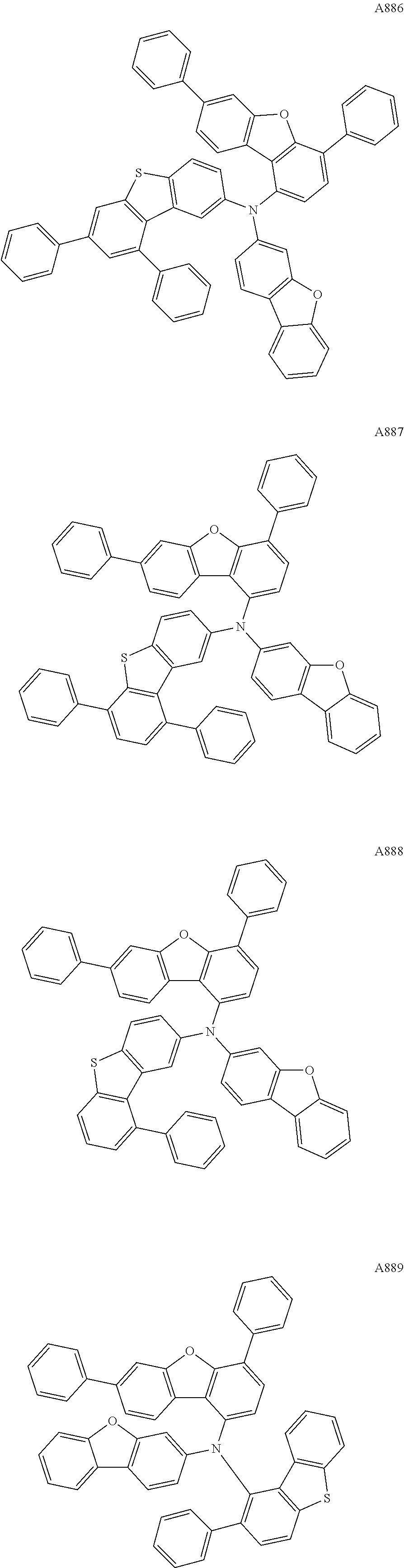 [SMILES: c1ccc(-c2ccc3c(c2)oc2c(-c4ccccc4)ccc(N(c4ccc5c(c4)oc4ccccc45)c4ccc5sc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5sc6c(-c7ccccc7)ccc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5sc6cccc(-c7ccccc7)c6c5c4)c23)cc1 ; c1ccc(-c2ccc3c(c2)oc2c(-c4ccccc4)ccc(N(c4ccc5c(c4)oc4ccccc45)c4c(-c5ccccc5)ccc5sc6ccccc6c45)c23)cc1]

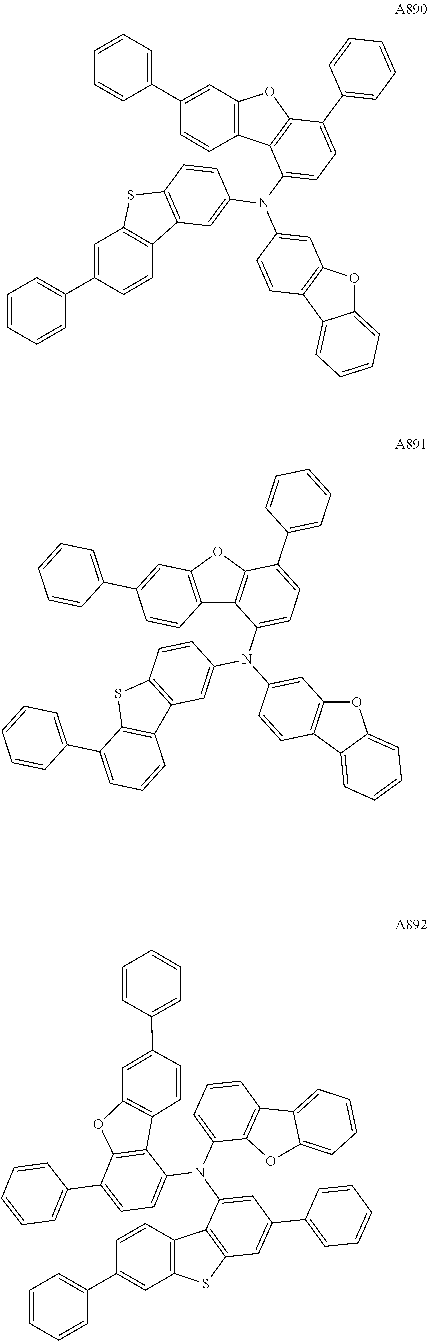 [SMILES: c1ccc(-c2ccc3c(c2)oc2c(-c4ccccc4)ccc(N(c4ccc5c(c4)oc4ccccc45)c4ccc5sc6cc(-c7ccccc7)ccc6c5c4)c23)cc1 ; c1ccc(-c2ccc3c(c2)oc2c(-c4ccccc4)ccc(N(c4ccc5c(c4)oc4ccccc45)c4ccc5sc6c(-c7ccccc7)cccc6c5c4)c23)cc1 ; c1ccc(-c2ccc3c(c2)oc2c(-c4ccccc4)ccc(N(c4cccc5c4oc4ccccc45)c4cc(-c5ccccc5)cc5sc6cc(-c7ccccc7)ccc6c45)c23)cc1]

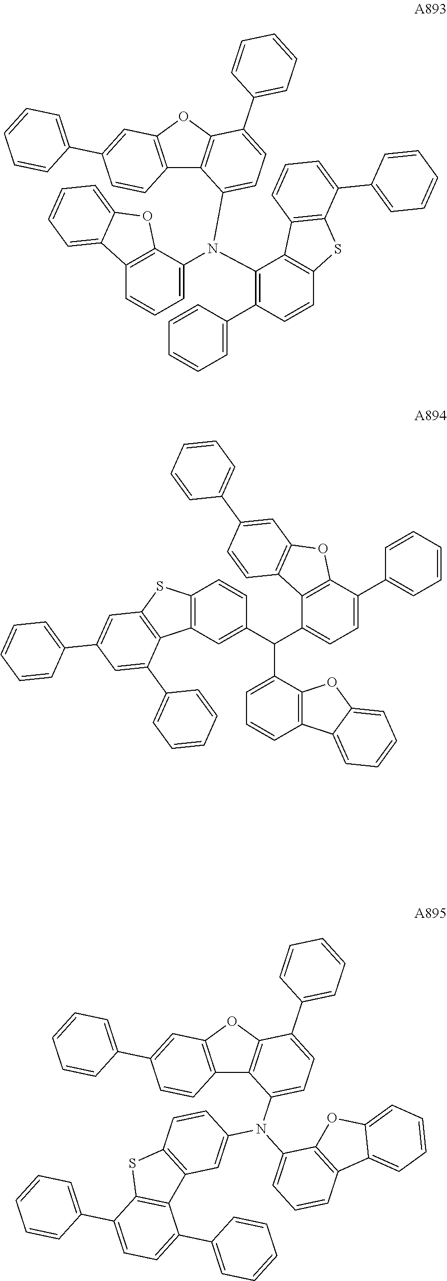 [SMILES: c1ccc(-c2ccc3c(c2)oc2c(-c4ccccc4)ccc(N(c4cccc5c4oc4ccccc45)c4c(-c5ccccc5)ccc5sc6c(-c7ccccc7)cccc6c45)c23)cc1 ; c1ccc(-c2ccc3c(c2)oc2c(-c4ccccc4)ccc(C(c4ccc5sc6cc(-c7ccccc7)cc(-c7ccccc7)c6c5c4)c4cccc5c4oc4ccccc45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5c4oc4ccccc45)c23)cc1]

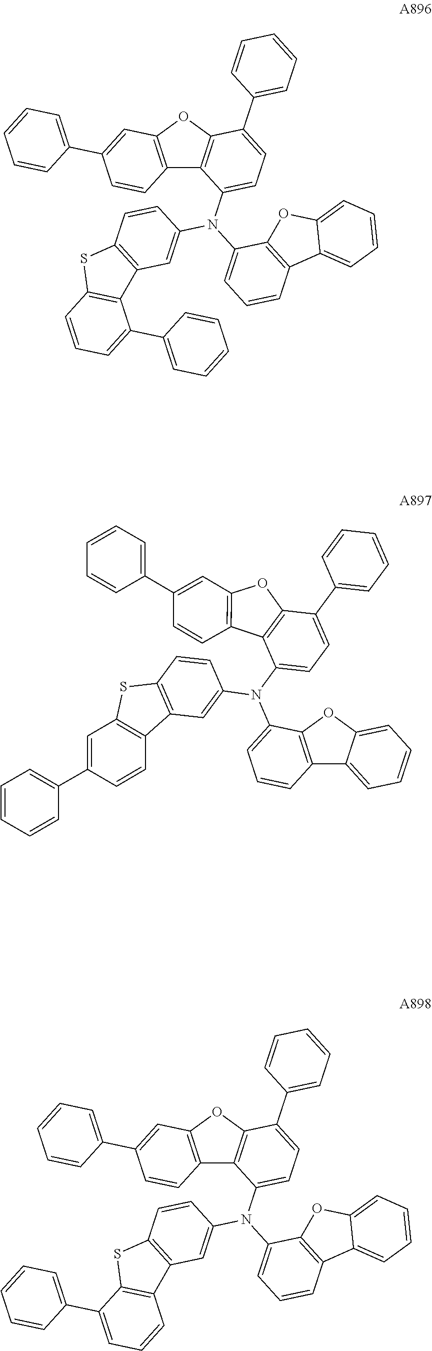 [SMILES: c1ccc(-c2ccc3c(c2)oc2c(-c4ccccc4)ccc(N(c4ccc5sc6cccc(-c7ccccc7)c6c5c4)c4cccc5c4oc4ccccc45)c23)cc1 ; c1ccc(-c2ccc3c(c2)oc2c(-c4ccccc4)ccc(N(c4ccc5sc6cc(-c7ccccc7)ccc6c5c4)c4cccc5c4oc4ccccc45)c23)cc1 ; c1ccc(-c2ccc3c(c2)oc2c(-c4ccccc4)ccc(N(c4ccc5sc6c(-c7ccccc7)cccc6c5c4)c4cccc5c4oc4ccccc45)c23)cc1]

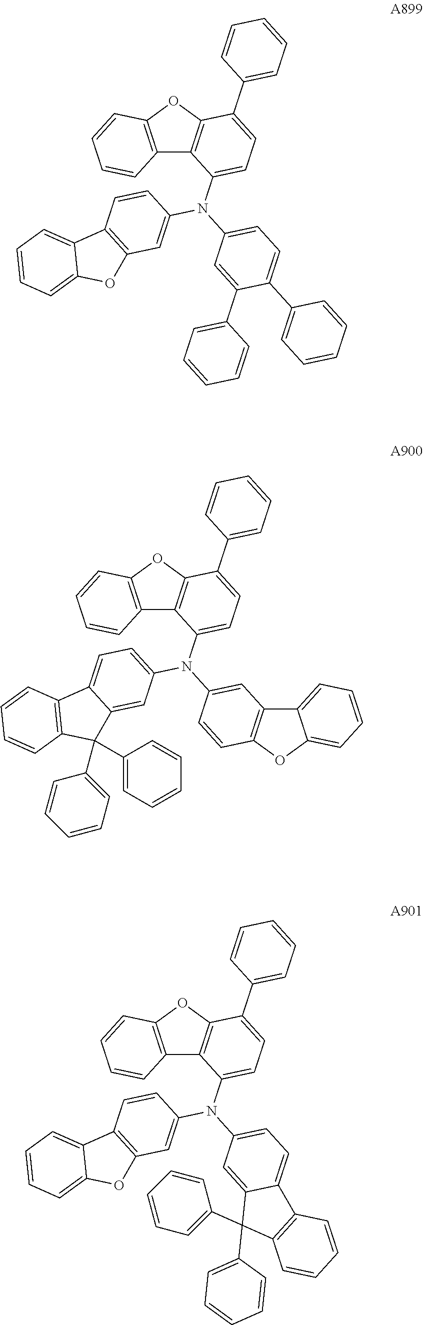 [SMILES: c1ccc(-c2ccc(N(c3ccc4c(c3)oc3ccccc34)c3ccc(-c4ccccc4)c4oc5ccccc5c34)cc2-c2ccccc2)cc1 ; c1ccc(-c2ccc(N(c3ccc4c(c3)C(c3ccccc3)(c3ccccc3)c3ccccc3-4)c3ccc4oc5ccccc5c4c3)c3c2oc2ccccc23)cc1 ; c1ccc(-c2ccc(N(c3ccc4c(c3)C(c3ccccc3)(c3ccccc3)c3ccccc3-4)c3ccc4c(c3)oc3ccccc34)c3c2oc2ccccc23)cc1]

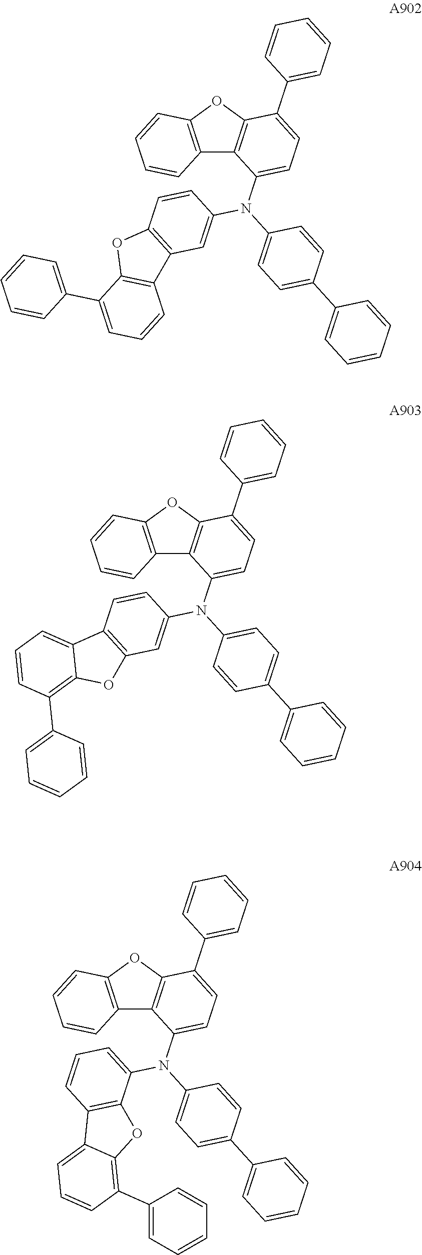 [SMILES: c1ccc(-c2ccc(N(c3ccc4oc5c(-c6ccccc6)cccc5c4c3)c3ccc(-c4ccccc4)c4oc5ccccc5c34)cc2)cc1 ; c1ccc(-c2ccc(N(c3ccc4c(c3)oc3c(-c5ccccc5)cccc34)c3ccc(-c4ccccc4)c4oc5ccccc5c34)cc2)cc1 ; c1ccc(-c2ccc(N(c3cccc4c3oc3c(-c5ccccc5)cccc34)c3ccc(-c4ccccc4)c4oc5ccccc5c34)cc2)cc1]

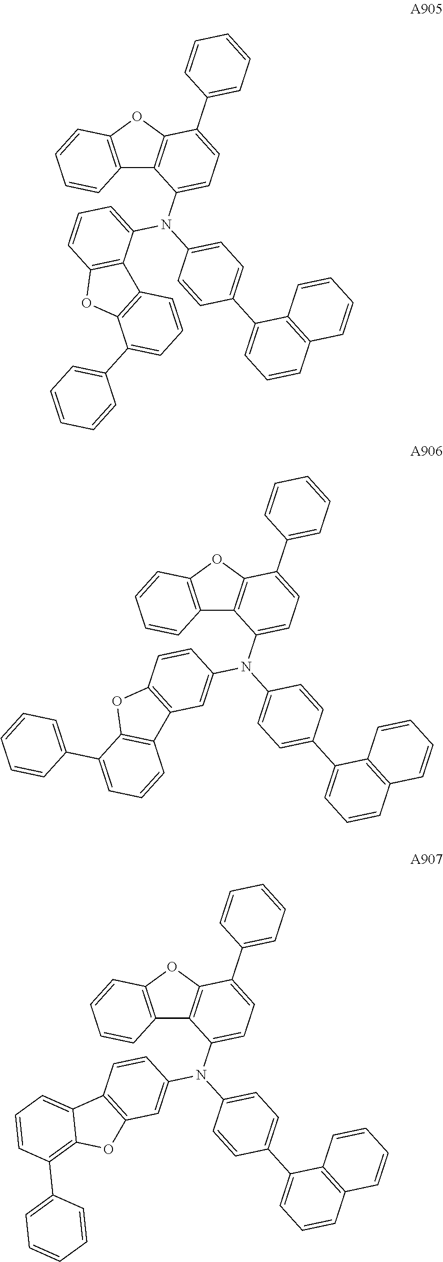 [SMILES: c1ccc(-c2cccc3c2oc2cccc(N(c4ccc(-c5cccc6ccccc56)cc4)c4ccc(-c5ccccc5)c5oc6ccccc6c45)c23)cc1 ; c1ccc(-c2cccc3c2oc2ccc(N(c4ccc(-c5cccc6ccccc56)cc4)c4ccc(-c5ccccc5)c5oc6ccccc6c45)cc23)cc1 ; c1ccc(-c2cccc3c2oc2cc(N(c4ccc(-c5cccc6ccccc56)cc4)c4ccc(-c5ccccc5)c5oc6ccccc6c45)ccc23)cc1]

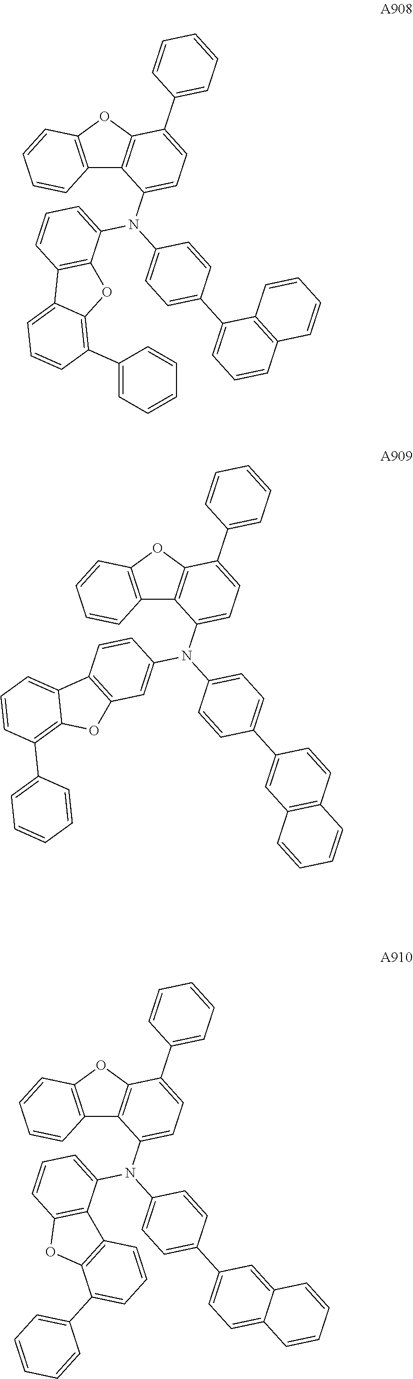 [SMILES: c1ccc(-c2cccc3c2oc2c(N(c4ccc(-c5cccc6ccccc56)cc4)c4ccc(-c5ccccc5)c5oc6ccccc6c45)cccc23)cc1 ; c1ccc(-c2cccc3c2oc2cc(N(c4ccc(-c5ccc6ccccc6c5)cc4)c4ccc(-c5ccccc5)c5oc6ccccc6c45)ccc23)cc1 ; c1ccc(-c2cccc3c2oc2cccc(N(c4ccc(-c5ccc6ccccc6c5)cc4)c4ccc(-c5ccccc5)c5oc6ccccc6c45)c23)cc1]

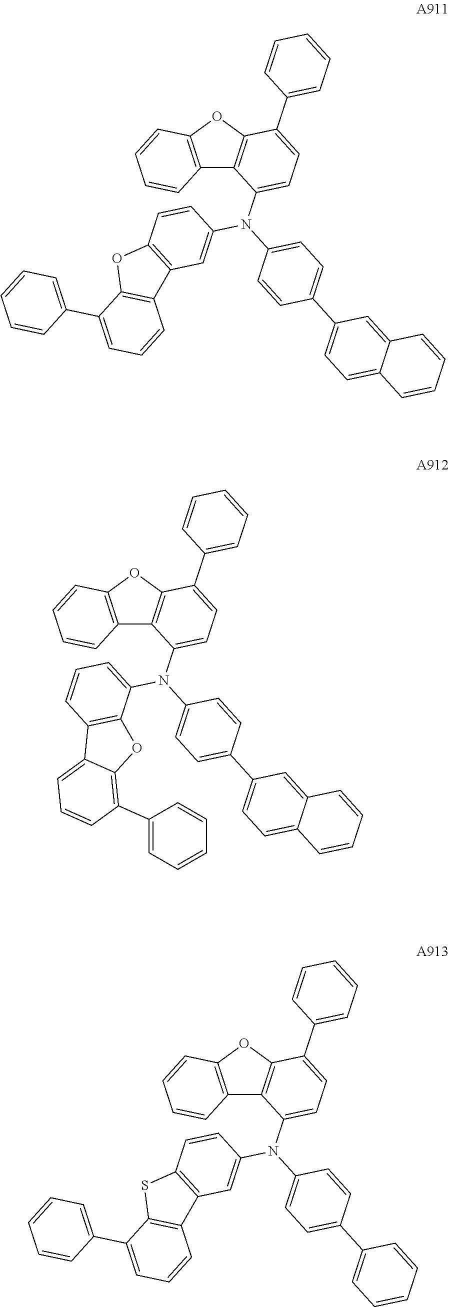 [SMILES: c1ccc(-c2cccc3c2oc2ccc(N(c4ccc(-c5ccc6ccccc6c5)cc4)c4ccc(-c5ccccc5)c5oc6ccccc6c45)cc23)cc1 ; c1ccc(-c2cccc3c2oc2c(N(c4ccc(-c5ccc6ccccc6c5)cc4)c4ccc(-c5ccccc5)c5oc6ccccc6c45)cccc23)cc1 ; c1ccc(-c2ccc(N(c3ccc4sc5c(-c6ccccc6)cccc5c4c3)c3ccc(-c4ccccc4)c4oc5ccccc5c34)cc2)cc1]

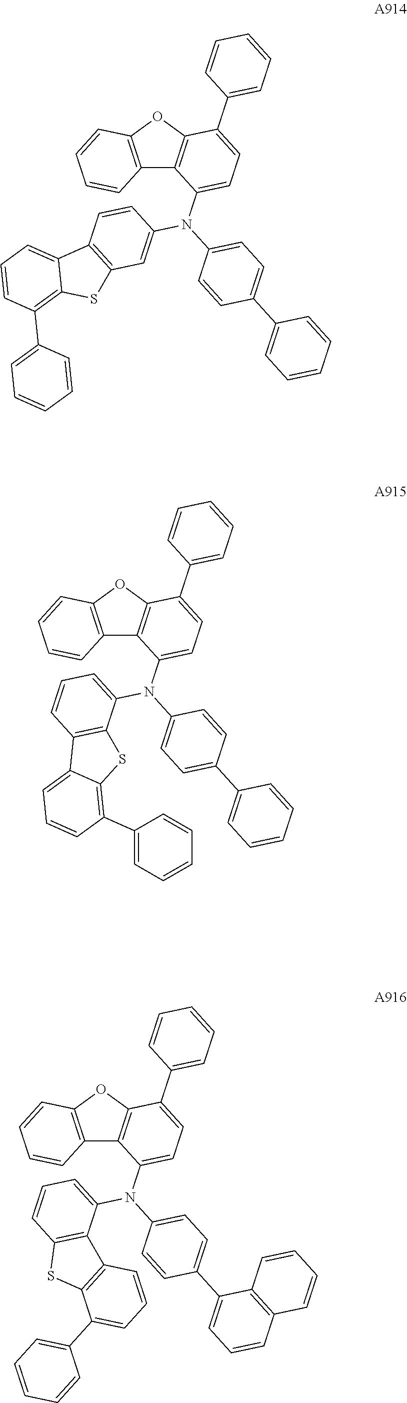 [SMILES: c1ccc(-c2ccc(N(c3ccc4c(c3)sc3c(-c5ccccc5)cccc34)c3ccc(-c4ccccc4)c4oc5ccccc5c34)cc2)cc1 ; c1ccc(-c2ccc(N(c3cccc4c3sc3c(-c5ccccc5)cccc34)c3ccc(-c4ccccc4)c4oc5ccccc5c34)cc2)cc1 ; c1ccc(-c2ccc(N(c3ccc(-c4cccc5ccccc45)cc3)c3cccc4sc5c(-c6ccccc6)cccc5c34)c3c2oc2ccccc23)cc1]

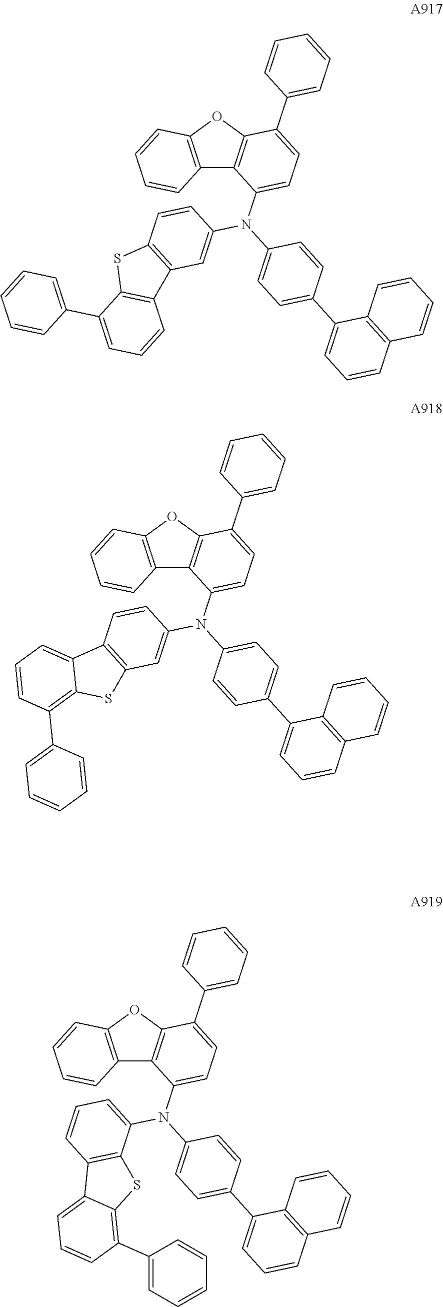 [SMILES: c1ccc(-c2ccc(N(c3ccc(-c4cccc5ccccc45)cc3)c3ccc4sc5c(-c6ccccc6)cccc5c4c3)c3c2oc2ccccc23)cc1 ; c1ccc(-c2ccc(N(c3ccc(-c4cccc5ccccc45)cc3)c3ccc4c(c3)sc3c(-c5ccccc5)cccc34)c3c2oc2ccccc23)cc1 ; c1ccc(-c2ccc(N(c3ccc(-c4cccc5ccccc45)cc3)c3cccc4c3sc3c(-c5ccccc5)cccc34)c3c2oc2ccccc23)cc1]

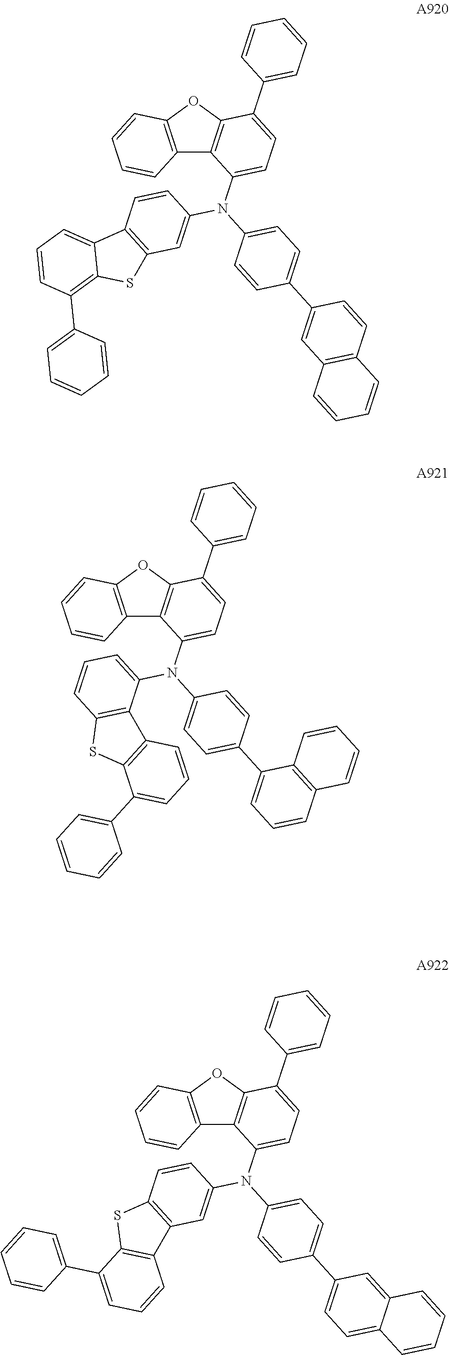 [SMILES: c1ccc(-c2ccc(N(c3ccc(-c4ccc5ccccc5c4)cc3)c3ccc4c(c3)sc3c(-c5ccccc5)cccc34)c3c2oc2ccccc23)cc1 ; c1ccc(-c2ccc(N(c3ccc(-c4cccc5ccccc45)cc3)c3cccc4sc5c(-c6ccccc6)cccc5c34)c3c2oc2ccccc23)cc1 ; c1ccc(-c2ccc(N(c3ccc(-c4ccc5ccccc5c4)cc3)c3ccc4sc5c(-c6ccccc6)cccc5c4c3)c3c2oc2ccccc23)cc1]

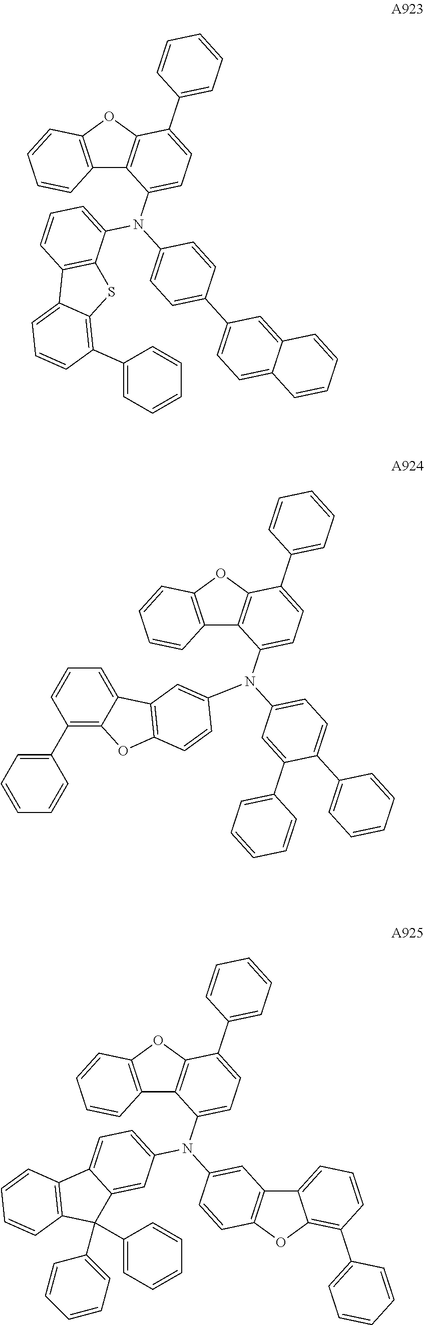 [SMILES: c1ccc(-c2ccc(N(c3ccc(-c4ccc5ccccc5c4)cc3)c3cccc4c3sc3c(-c5ccccc5)cccc34)c3c2oc2ccccc23)cc1 ; c1ccc(-c2ccc(N(c3ccc4oc5c(-c6ccccc6)cccc5c4c3)c3ccc(-c4ccccc4)c4oc5ccccc5c34)cc2-c2ccccc2)cc1 ; c1ccc(-c2cccc3c2oc2ccc(N(c4ccc5c(c4)C(c4ccccc4)(c4ccccc4)c4ccccc4-5)c4ccc(-c5ccccc5)c5oc6ccccc6c45)cc23)cc1]

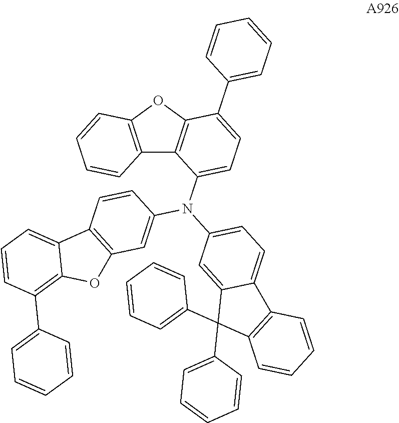 [SMILES: c1ccc(-c2cccc3c2oc2cc(N(c4ccc5c(c4)C(c4ccccc4)(c4ccccc4)c4ccccc4-5)c4ccc(-c5ccccc5)c5oc6ccccc6c45)ccc23)cc1]

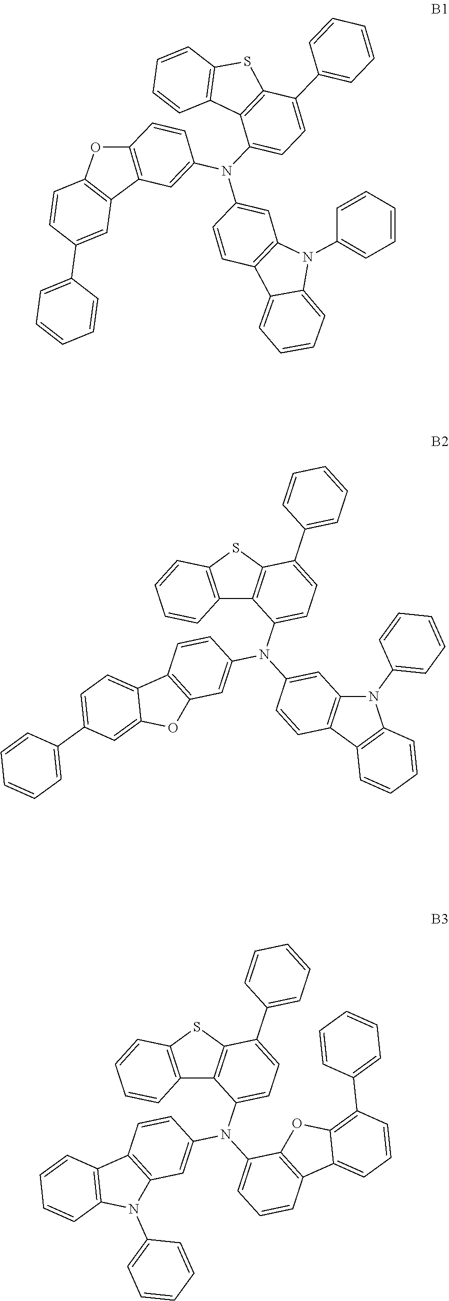 [SMILES: c1ccc(-c2ccc3oc4ccc(N(c5ccc6c7ccccc7n(-c7ccccc7)c6c5)c5ccc(-c6ccccc6)c6sc7ccccc7c56)cc4c3c2)cc1 ; c1ccc(-c2ccc3c(c2)oc2cc(N(c4ccc5c6ccccc6n(-c6ccccc6)c5c4)c4ccc(-c5ccccc5)c5sc6ccccc6c45)ccc23)cc1 ; c1ccc(-c2cccc3c2oc2c(N(c4ccc5c6ccccc6n(-c6ccccc6)c5c4)c4ccc(-c5ccccc5)c5sc6ccccc6c45)cccc23)cc1]

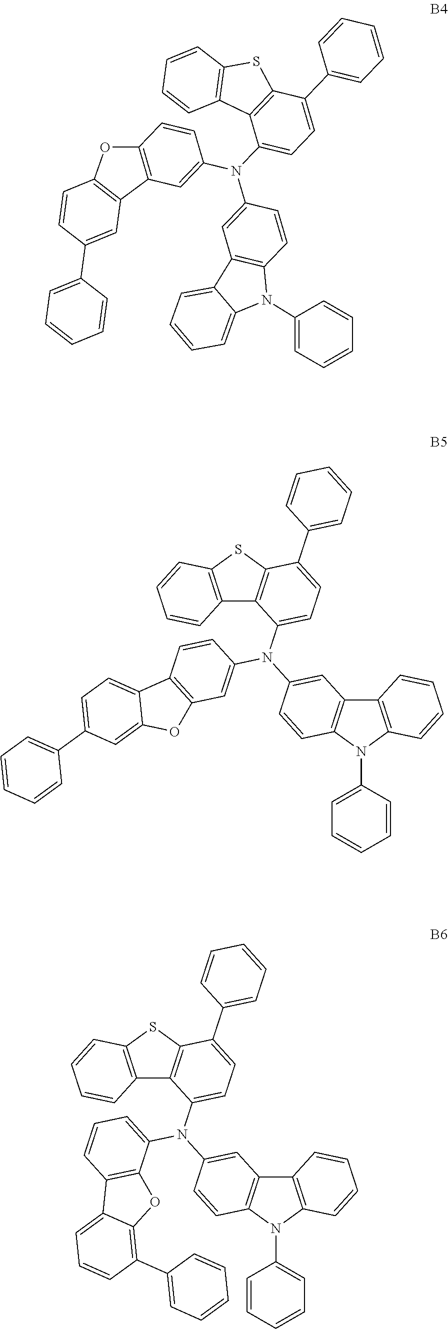 [SMILES: c1ccc(-c2ccc3oc4ccc(N(c5ccc6c(c5)c5ccccc5n6-c5ccccc5)c5ccc(-c6ccccc6)c6sc7ccccc7c56)cc4c3c2)cc1 ; c1ccc(-c2ccc3c(c2)oc2cc(N(c4ccc5c(c4)c4ccccc4n5-c4ccccc4)c4ccc(-c5ccccc5)c5sc6ccccc6c45)ccc23)cc1 ; c1ccc(-c2cccc3c2oc2c(N(c4ccc5c(c4)c4ccccc4n5-c4ccccc4)c4ccc(-c5ccccc5)c5sc6ccccc6c45)cccc23)cc1]

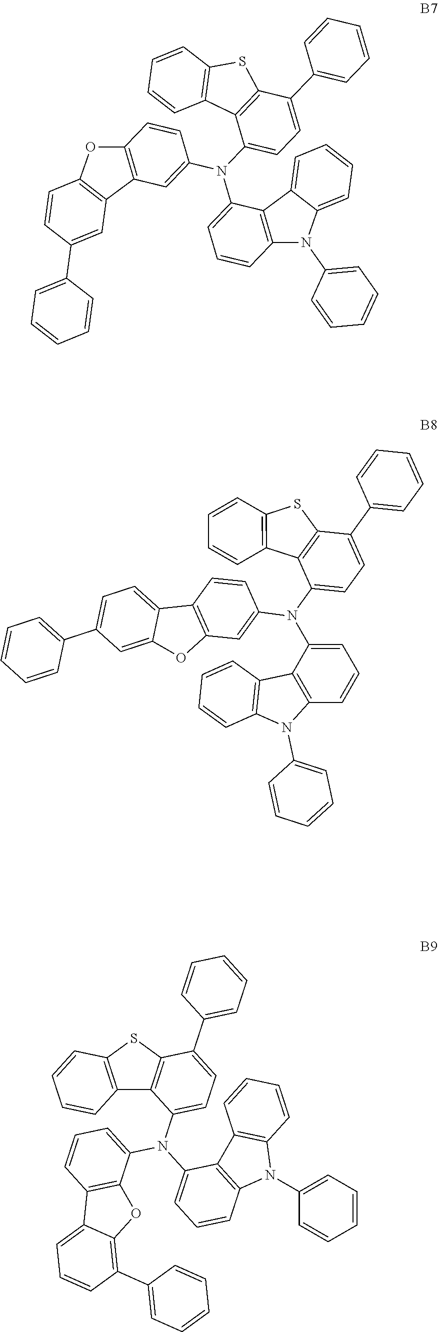 [SMILES: c1ccc(-c2ccc3oc4ccc(N(c5ccc(-c6ccccc6)c6sc7ccccc7c56)c5cccc6c5c5ccccc5n6-c5ccccc5)cc4c3c2)cc1 ; c1ccc(-c2ccc3c(c2)oc2cc(N(c4ccc(-c5ccccc5)c5sc6ccccc6c45)c4cccc5c4c4ccccc4n5-c4ccccc4)ccc23)cc1 ; c1ccc(-c2cccc3c2oc2c(N(c4ccc(-c5ccccc5)c5sc6ccccc6c45)c4cccc5c4c4ccccc4n5-c4ccccc4)cccc23)cc1]

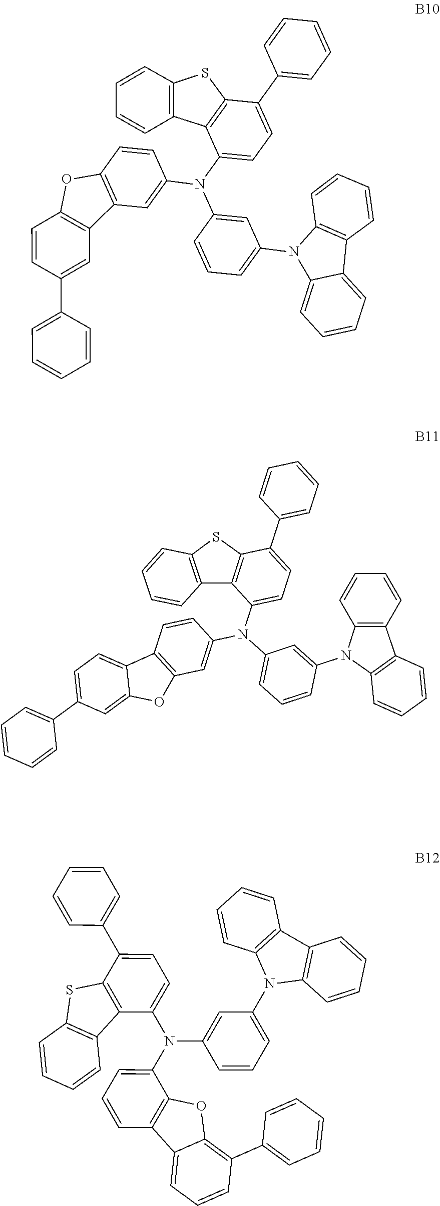 [SMILES: c1ccc(-c2ccc3oc4ccc(N(c5cccc(-n6c7ccccc7c7ccccc76)c5)c5ccc(-c6ccccc6)c6sc7ccccc7c56)cc4c3c2)cc1 ; c1ccc(-c2ccc3c(c2)oc2cc(N(c4cccc(-n5c6ccccc6c6ccccc65)c4)c4ccc(-c5ccccc5)c5sc6ccccc6c45)ccc23)cc1 ; c1ccc(-c2cccc3c2oc2c(N(c4cccc(-n5c6ccccc6c6ccccc65)c4)c4ccc(-c5ccccc5)c5sc6ccccc6c45)cccc23)cc1]

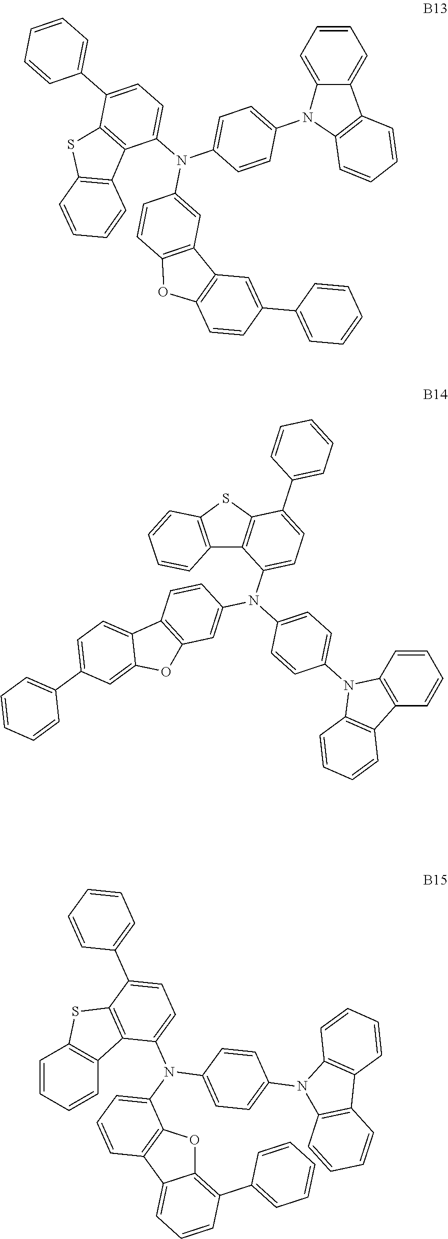 [SMILES: c1ccc(-c2ccc3oc4ccc(N(c5ccc(-n6c7ccccc7c7ccccc76)cc5)c5ccc(-c6ccccc6)c6sc7ccccc7c56)cc4c3c2)cc1 ; c1ccc(-c2ccc3c(c2)oc2cc(N(c4ccc(-n5c6ccccc6c6ccccc65)cc4)c4ccc(-c5ccccc5)c5sc6ccccc6c45)ccc23)cc1 ; c1ccc(-c2cccc3c2oc2c(N(c4ccc(-n5c6ccccc6c6ccccc65)cc4)c4ccc(-c5ccccc5)c5sc6ccccc6c45)cccc23)cc1]

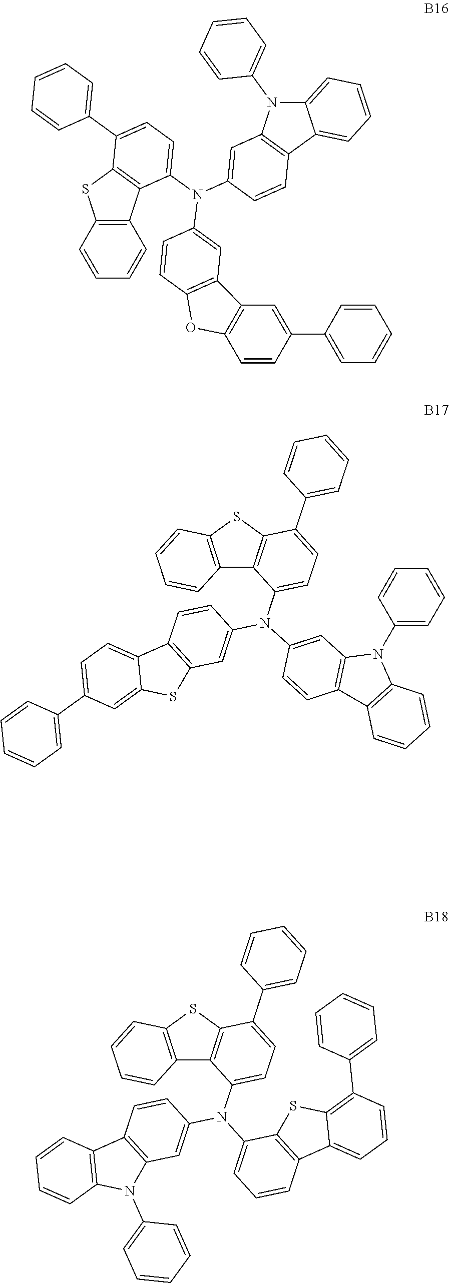 [SMILES: c1ccc(-c2ccc3oc4ccc(N(c5ccc6c7ccccc7n(-c7ccccc7)c6c5)c5ccc(-c6ccccc6)c6sc7ccccc7c56)cc4c3c2)cc1 ; c1ccc(-c2ccc3c(c2)sc2cc(N(c4ccc5c6ccccc6n(-c6ccccc6)c5c4)c4ccc(-c5ccccc5)c5sc6ccccc6c45)ccc23)cc1 ; c1ccc(-c2cccc3c2sc2c(N(c4ccc5c6ccccc6n(-c6ccccc6)c5c4)c4ccc(-c5ccccc5)c5sc6ccccc6c45)cccc23)cc1]

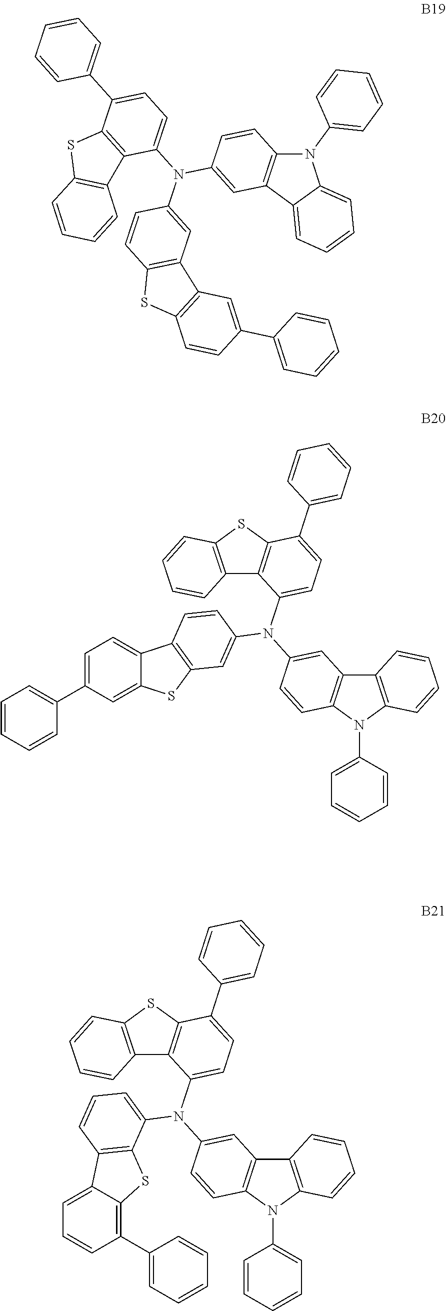 [SMILES: c1ccc(-c2ccc3sc4ccc(N(c5ccc6c(c5)c5ccccc5n6-c5ccccc5)c5ccc(-c6ccccc6)c6sc7ccccc7c56)cc4c3c2)cc1 ; c1ccc(-c2ccc3c(c2)sc2cc(N(c4ccc5c(c4)c4ccccc4n5-c4ccccc4)c4ccc(-c5ccccc5)c5sc6ccccc6c45)ccc23)cc1 ; c1ccc(-c2cccc3c2sc2c(N(c4ccc5c(c4)c4ccccc4n5-c4ccccc4)c4ccc(-c5ccccc5)c5sc6ccccc6c45)cccc23)cc1]

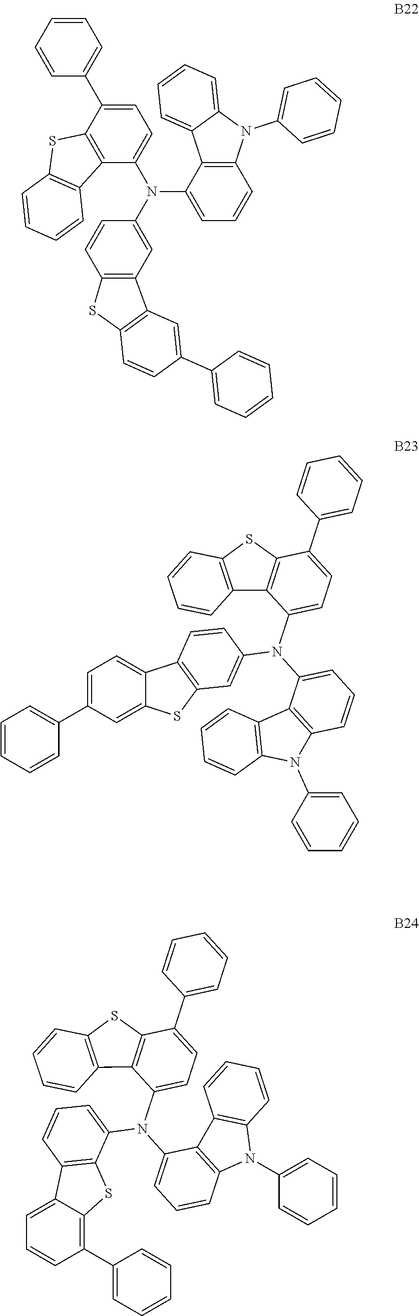 [SMILES: c1ccc(-c2ccc3sc4ccc(N(c5ccc(-c6ccccc6)c6sc7ccccc7c56)c5cccc6c5c5ccccc5n6-c5ccccc5)cc4c3c2)cc1 ; c1ccc(-c2ccc3c(c2)sc2cc(N(c4ccc(-c5ccccc5)c5sc6ccccc6c45)c4cccc5c4c4ccccc4n5-c4ccccc4)ccc23)cc1 ; c1ccc(-c2cccc3c2sc2c(N(c4ccc(-c5ccccc5)c5sc6ccccc6c45)c4cccc5c4c4ccccc4n5-c4ccccc4)cccc23)cc1]

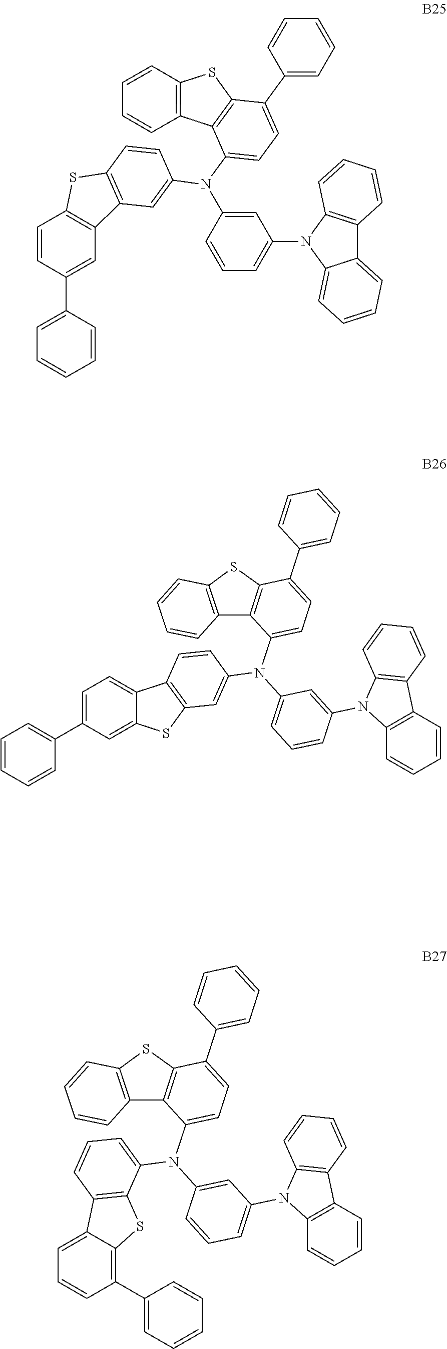 [SMILES: c1ccc(-c2ccc3sc4ccc(N(c5cccc(-n6c7ccccc7c7ccccc76)c5)c5ccc(-c6ccccc6)c6sc7ccccc7c56)cc4c3c2)cc1 ; c1ccc(-c2ccc3c(c2)sc2cc(N(c4cccc(-n5c6ccccc6c6ccccc65)c4)c4ccc(-c5ccccc5)c5sc6ccccc6c45)ccc23)cc1 ; c1ccc(-c2cccc3c2sc2c(N(c4cccc(-n5c6ccccc6c6ccccc65)c4)c4ccc(-c5ccccc5)c5sc6ccccc6c45)cccc23)cc1]

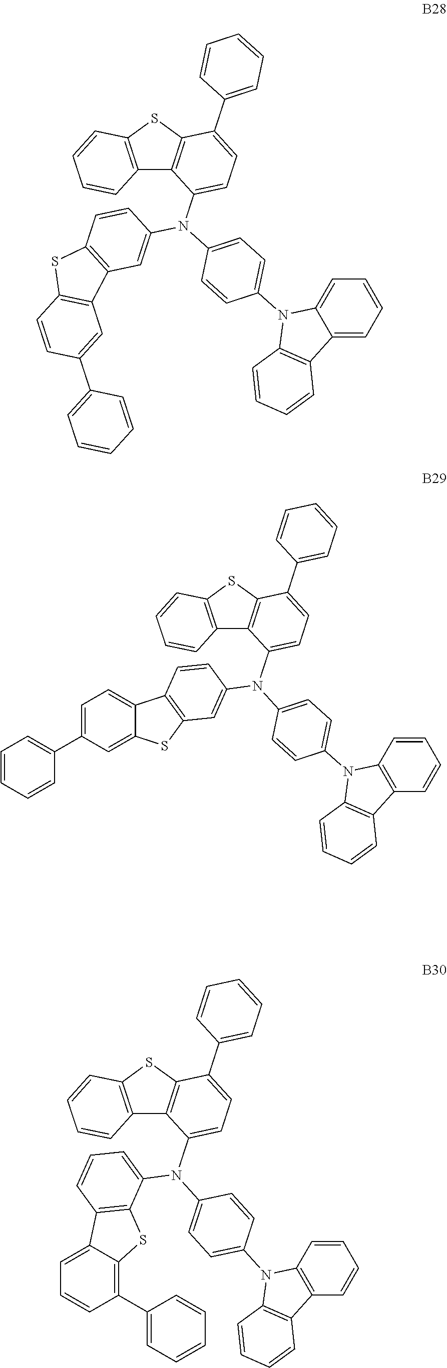 [SMILES: c1ccc(-c2ccc3sc4ccc(N(c5ccc(-n6c7ccccc7c7ccccc76)cc5)c5ccc(-c6ccccc6)c6sc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5sc6ccccc6c45)ccc23)cc1 ; c1ccc(-c2cccc3c2sc2c(N(c4ccc(-n5c6ccccc6c6ccccc65)cc4)c4ccc(-c5ccccc5)c5sc6ccccc6c45)cccc23)cc1]

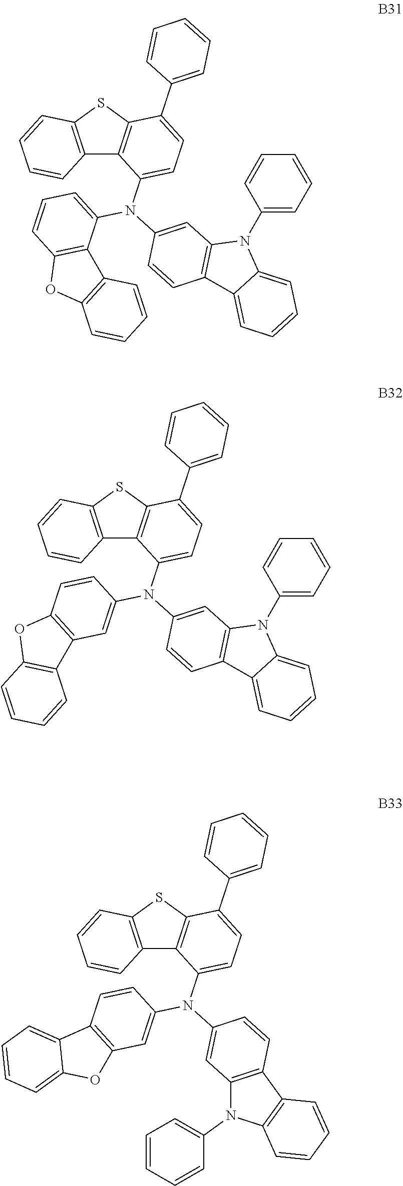 [SMILES: c1ccc(-c2ccc(N(c3ccc4c5ccccc5n(-c5ccccc5)c4c3)c3cccc4oc5ccccc5c34)c3c2sc2ccccc23)cc1 ; c1ccc(-c2ccc(N(c3ccc4oc5ccccc5c4c3)c3ccc4c5ccccc5n(-c5ccccc5)c4c3)c3c2sc2ccccc23)cc1 ; c1ccc(-c2ccc(N(c3ccc4c(c3)oc3ccccc34)c3ccc4c5ccccc5n(-c5ccccc5)c4c3)c3c2sc2ccccc23)cc1]

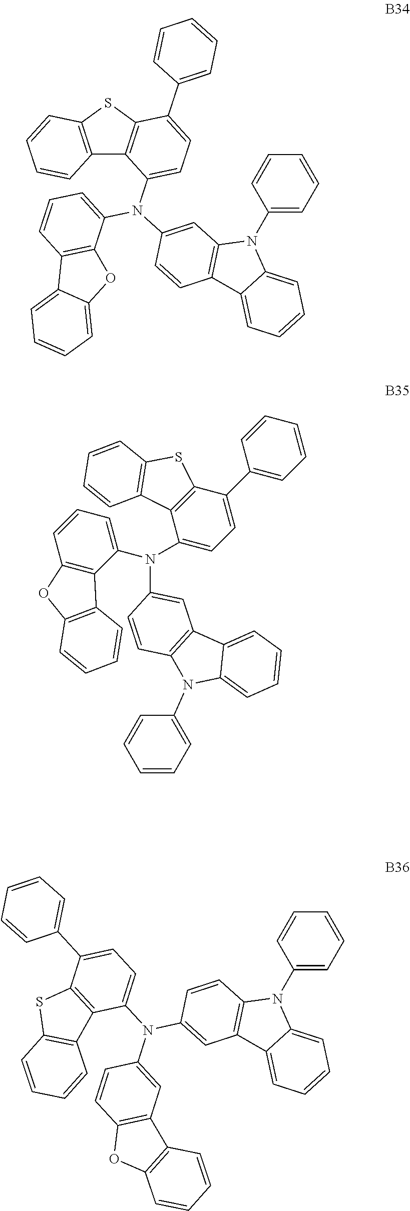 [SMILES: c1ccc(-c2ccc(N(c3ccc4c5ccccc5n(-c5ccccc5)c4c3)c3cccc4c3oc3ccccc34)c3c2sc2ccccc23)cc1 ; c1ccc(-c2ccc(N(c3ccc4c(c3)c3ccccc3n4-c3ccccc3)c3cccc4oc5ccccc5c34)c3c2sc2ccccc23)cc1 ; c1ccc(-c2ccc(N(c3ccc4oc5ccccc5c4c3)c3ccc4c(c3)c3ccccc3n4-c3ccccc3)c3c2sc2ccccc23)cc1]

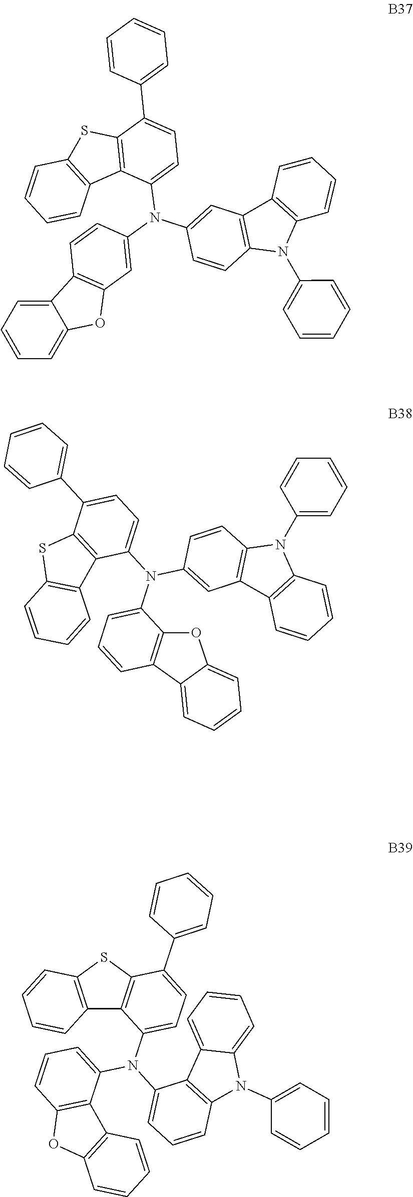 [SMILES: c1ccc(-c2ccc(N(c3ccc4c(c3)oc3ccccc34)c3ccc4c(c3)c3ccccc3n4-c3ccccc3)c3c2sc2ccccc23)cc1 ; c1ccc(-c2ccc(N(c3ccc4c(c3)c3ccccc3n4-c3ccccc3)c3cccc4c3oc3ccccc34)c3c2sc2ccccc23)cc1 ; c1ccc(-c2ccc(N(c3cccc4oc5ccccc5c34)c3cccc4c3c3ccccc3n4-c3ccccc3)c3c2sc2ccccc23)cc1]

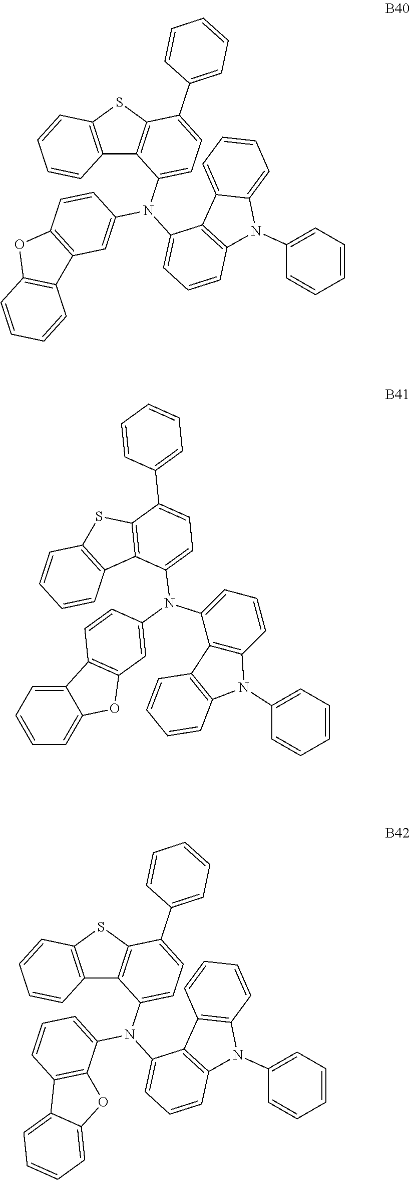 [SMILES: c1ccc(-c2ccc(N(c3ccc4oc5ccccc5c4c3)c3cccc4c3c3ccccc3n4-c3ccccc3)c3c2sc2ccccc23)cc1 ; c1ccc(-c2ccc(N(c3ccc4c(c3)oc3ccccc34)c3cccc4c3c3ccccc3n4-c3ccccc3)c3c2sc2ccccc23)cc1 ; c1ccc(-c2ccc(N(c3cccc4c3oc3ccccc34)c3cccc4c3c3ccccc3n4-c3ccccc3)c3c2sc2ccccc23)cc1]

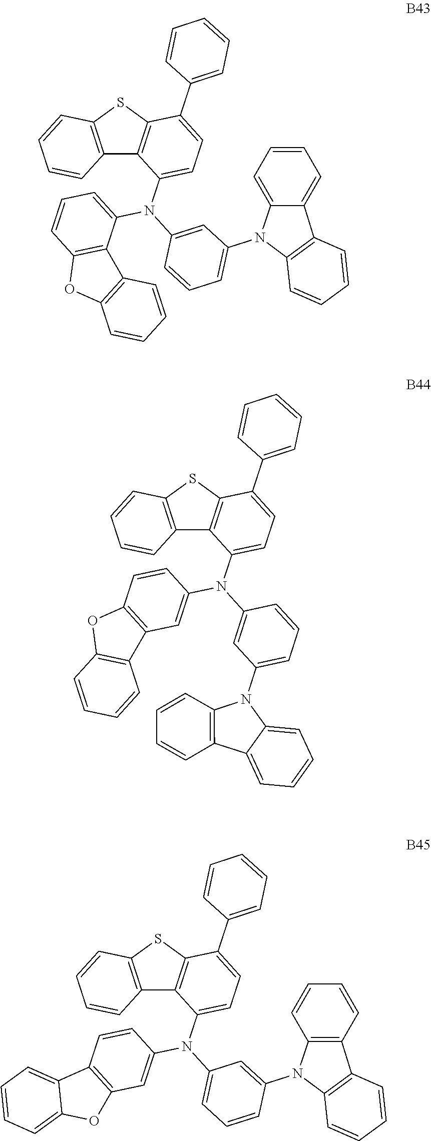 [SMILES: c1ccc(-c2ccc(N(c3cccc(-n4c5ccccc5c5ccccc54)c3)c3cccc4oc5ccccc5c34)c3c2sc2ccccc23)cc1 ; c1ccc(-c2ccc(N(c3cccc(-n4c5ccccc5c5ccccc54)c3)c3ccc4oc5ccccc5c4c3)c3c2sc2ccccc23)cc1 ; c1ccc(-c2ccc(N(c3cccc(-n4c5ccccc5c5ccccc54)c3)c3ccc4c(c3)oc3ccccc34)c3c2sc2ccccc23)cc1]

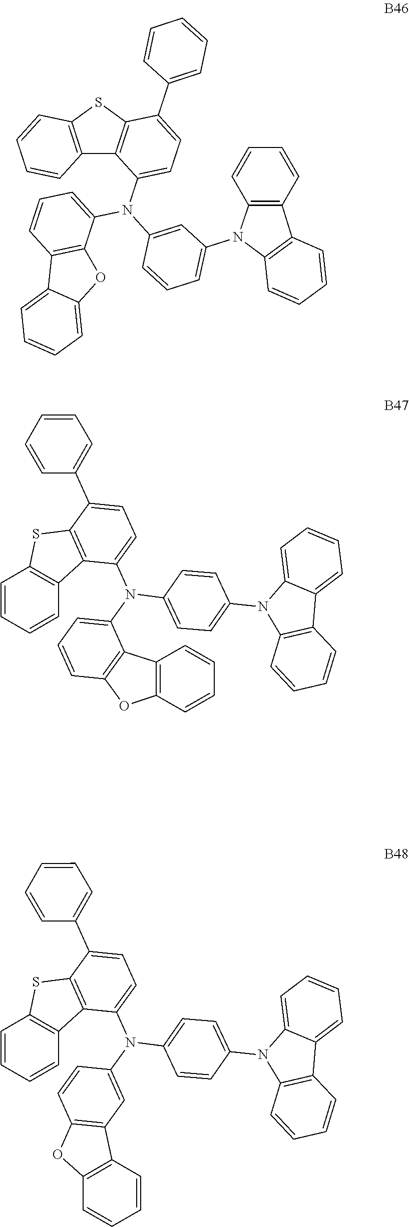 [SMILES: c1ccc(-c2ccc(N(c3cccc(-n4c5ccccc5c5ccccc54)c3)c3cccc4c3oc3ccccc34)c3c2sc2ccccc23)cc1 ; c1ccc(-c2ccc(N(c3ccc(-n4c5ccccc5c5ccccc54)cc3)c3cccc4oc5ccccc5c34)c3c2sc2ccccc23)cc1 ; c1ccc(-c2ccc(N(c3ccc(-n4c5ccccc5c5ccccc54)cc3)c3ccc4oc5ccccc5c4c3)c3c2sc2ccccc23)cc1]

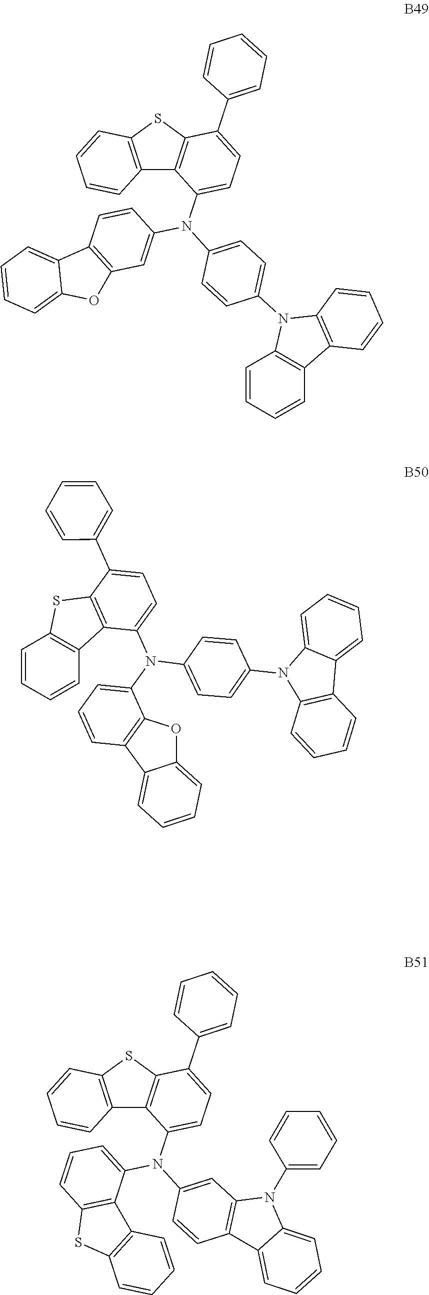 [SMILES: c1ccc(-c2ccc(N(c3ccc(-n4c5ccccc5c5ccccc54)cc3)c3ccc4c(c3)oc3ccccc34)c3c2sc2ccccc23)cc1 ; c1ccc(-c2ccc(N(c3ccc(-n4c5ccccc5c5ccccc54)cc3)c3cccc4c3oc3ccccc34)c3c2sc2ccccc23)cc1 ; c1ccc(-c2ccc(N(c3ccc4c5ccccc5n(-c5ccccc5)c4c3)c3cccc4sc5ccccc5c34)c3c2sc2ccccc23)cc1]

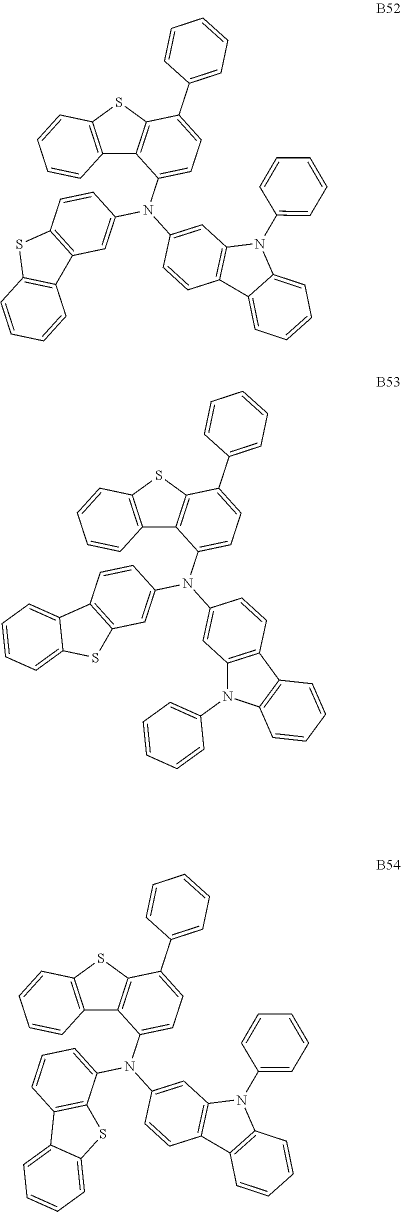 [SMILES: c1ccc(-c2ccc(N(c3ccc4sc5ccccc5c4c3)c3ccc4c5ccccc5n(-c5ccccc5)c4c3)c3c2sc2ccccc23)cc1 ; c1ccc(-c2ccc(N(c3ccc4c(c3)sc3ccccc34)c3ccc4c5ccccc5n(-c5ccccc5)c4c3)c3c2sc2ccccc23)cc1 ; c1ccc(-c2ccc(N(c3ccc4c5ccccc5n(-c5ccccc5)c4c3)c3cccc4c3sc3ccccc34)c3c2sc2ccccc23)cc1]

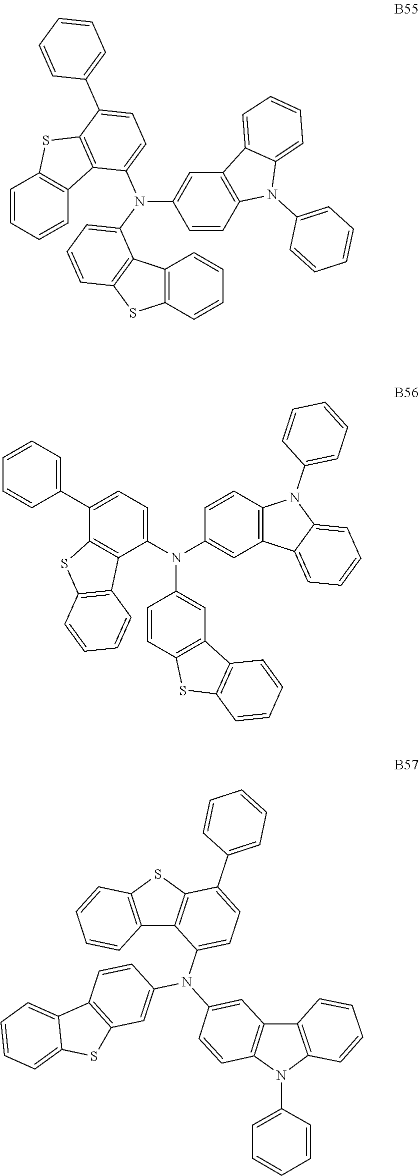 [SMILES: c1ccc(-c2ccc(N(c3ccc4c(c3)c3ccccc3n4-c3ccccc3)c3cccc4sc5ccccc5c34)c3c2sc2ccccc23)cc1 ; c1ccc(-c2ccc(N(c3ccc4sc5ccccc5c4c3)c3ccc4c(c3)c3ccccc3n4-c3ccccc3)c3c2sc2ccccc23)cc1 ; c1ccc(-c2ccc(N(c3ccc4c(c3)sc3ccccc34)c3ccc4c(c3)c3ccccc3n4-c3ccccc3)c3c2sc2ccccc23)cc1]

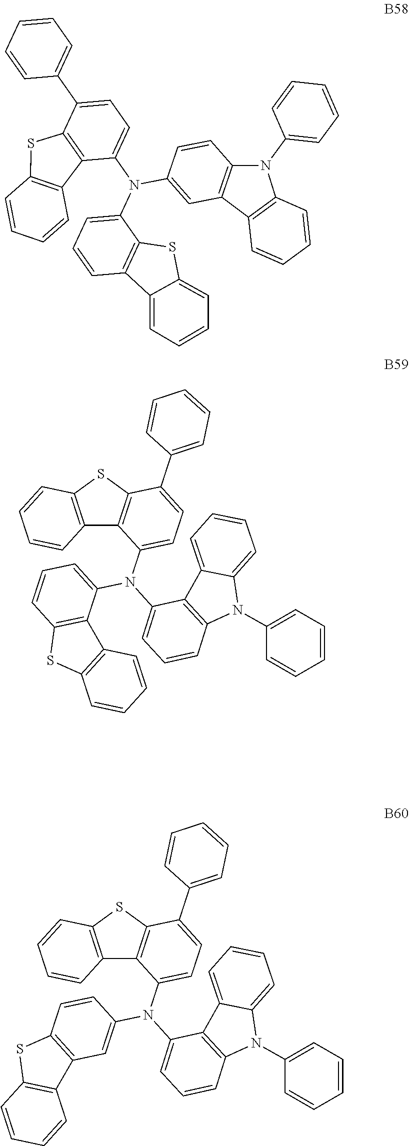

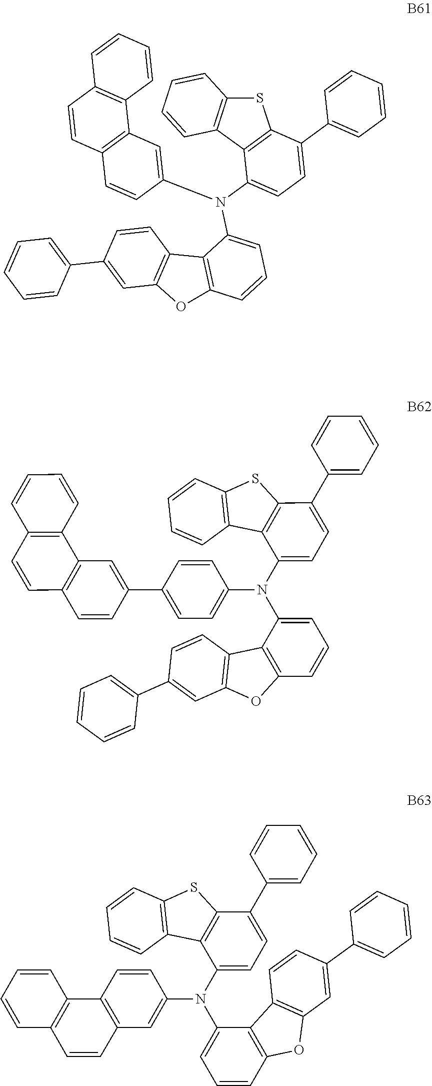 [SMILES: c1ccc(-c2ccc3c(c2)oc2cccc(N(c4ccc5ccc6ccccc6c5c4)c4ccc(-c5ccccc5)c5sc6ccccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5sc6ccccc6c45)c23)cc1 ; c1ccc(-c2ccc3c(c2)oc2cccc(N(c4ccc5c(ccc6ccccc65)c4)c4ccc(-c5ccccc5)c5sc6ccccc6c45)c23)cc1]

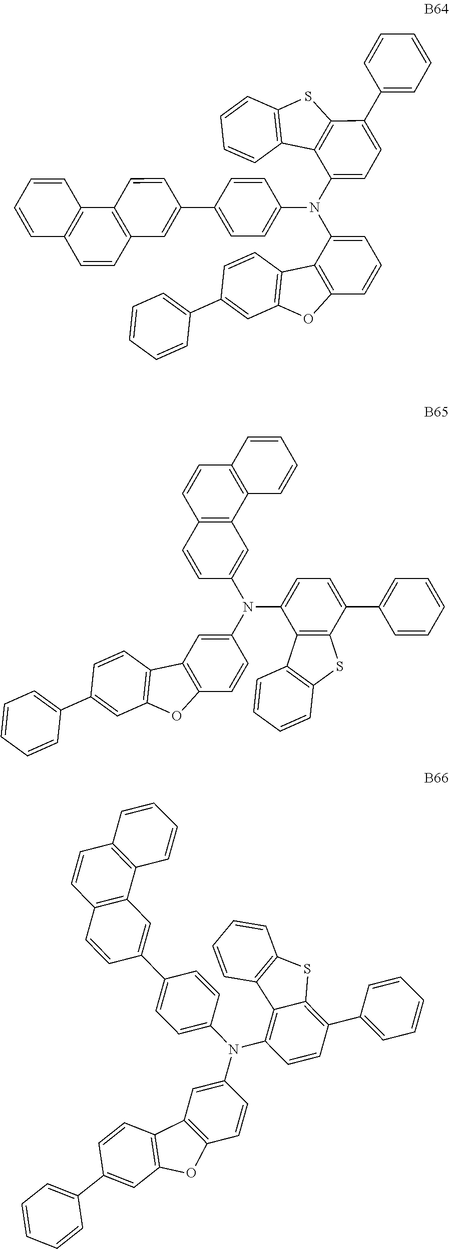 [SMILES: c1ccc(-c2ccc3c(c2)oc2cccc(N(c4ccc(-c5ccc6c(ccc7ccccc76)c5)cc4)c4ccc(-c5ccccc5)c5sc6ccccc6c45)c23)cc1 ; c1ccc(-c2ccc3c(c2)oc2ccc(N(c4ccc5ccc6ccccc6c5c4)c4ccc(-c5ccccc5)c5sc6ccccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5sc6ccccc6c45)cc23)cc1]

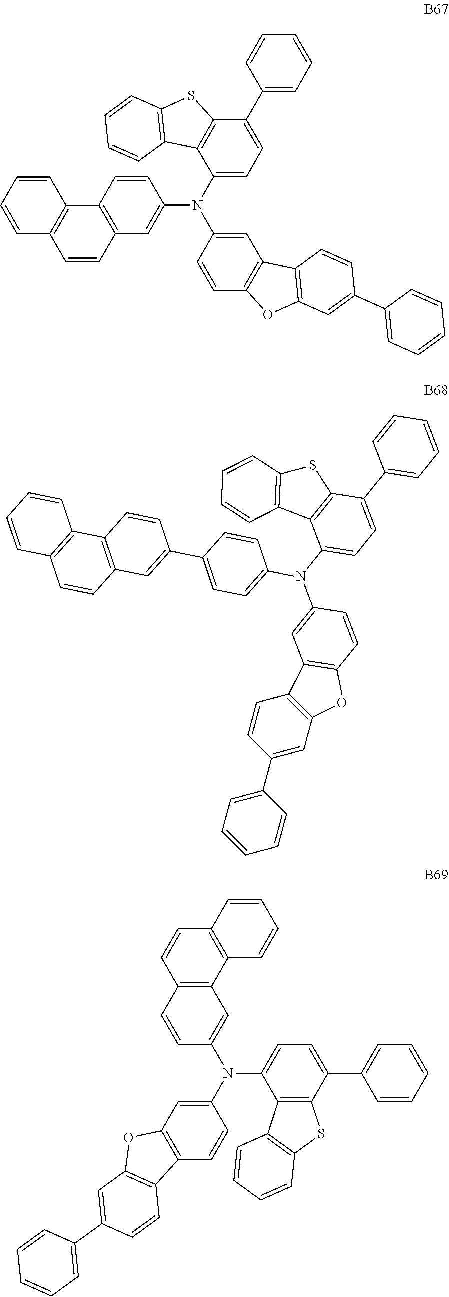 [SMILES: c1ccc(-c2ccc3c(c2)oc2ccc(N(c4ccc5c(ccc6ccccc65)c4)c4ccc(-c5ccccc5)c5sc6ccccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5sc6ccccc6c45)cc23)cc1 ; c1ccc(-c2ccc3c(c2)oc2cc(N(c4ccc5ccc6ccccc6c5c4)c4ccc(-c5ccccc5)c5sc6ccccc6c45)ccc23)cc1]

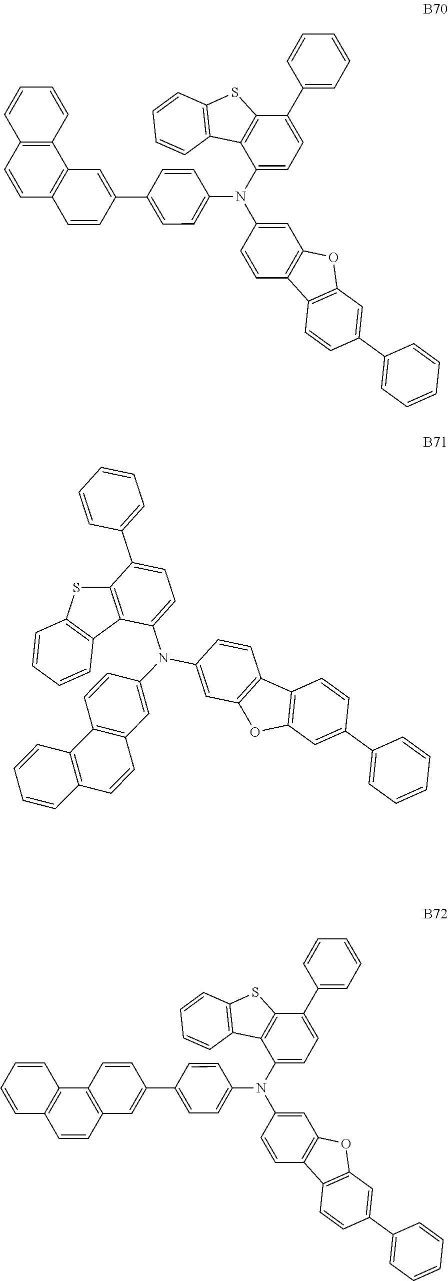 [SMILES: c1ccc(-c2ccc3c(c2)oc2cc(N(c4ccc(-c5ccc6ccc7ccccc7c6c5)cc4)c4ccc(-c5ccccc5)c5sc6ccccc6c45)ccc23)cc1 ; c1ccc(-c2ccc3c(c2)oc2cc(N(c4ccc5c(ccc6ccccc65)c4)c4ccc(-c5ccccc5)c5sc6ccccc6c45)ccc23)cc1 ; c1ccc(-c2ccc3c(c2)oc2cc(N(c4ccc(-c5ccc6c(ccc7ccccc76)c5)cc4)c4ccc(-c5ccccc5)c5sc6ccccc6c45)ccc23)cc1]

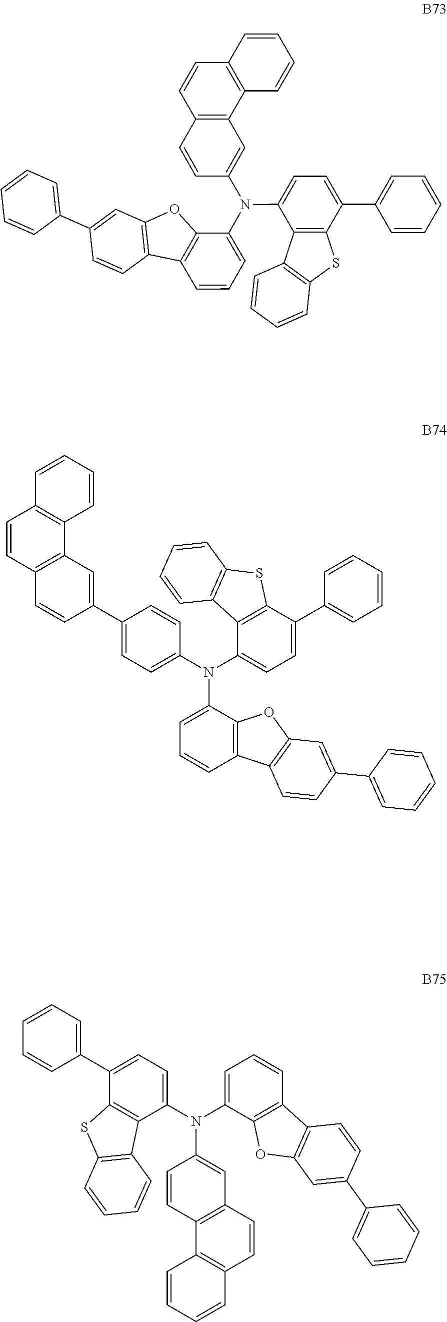 [SMILES: c1ccc(-c2ccc3c(c2)oc2c(N(c4ccc5ccc6ccccc6c5c4)c4ccc(-c5ccccc5)c5sc6ccccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)oc2c(N(c4ccc5c(ccc6ccccc65)c4)c4ccc(-c5ccccc5)c5sc6ccccc6c45)cccc23)cc1]

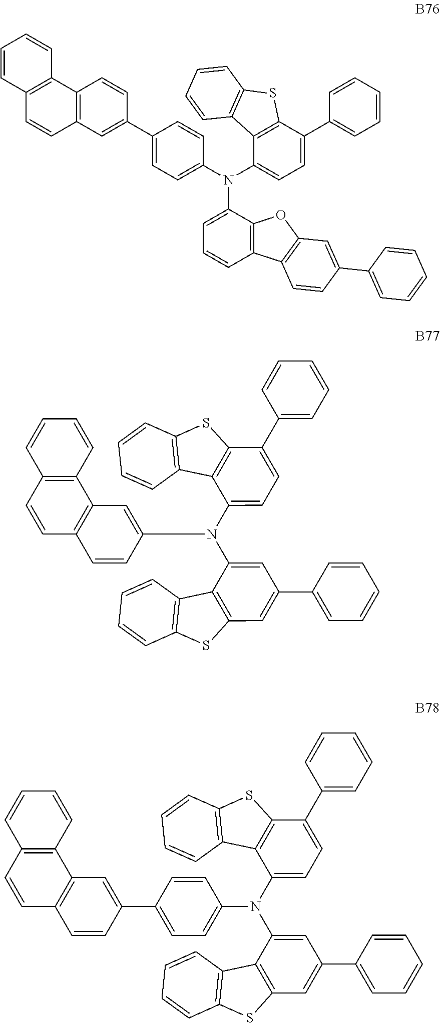 [SMILES: c1ccc(-c2ccc3c(c2)oc2c(N(c4ccc(-c5ccc6c(ccc7ccccc76)c5)cc4)c4ccc(-c5ccccc5)c5sc6ccccc6c45)cccc23)cc1 ; c1ccc(-c2cc(N(c3ccc4ccc5ccccc5c4c3)c3ccc(-c4ccccc4)c4sc5ccccc5c34)c3c(c2)sc2ccccc23)cc1 ; c1ccc(-c2cc(N(c3ccc(-c4ccc5ccc6ccccc6c5c4)cc3)c3ccc(-c4ccccc4)c4sc5ccccc5c34)c3c(c2)sc2ccccc23)cc1]

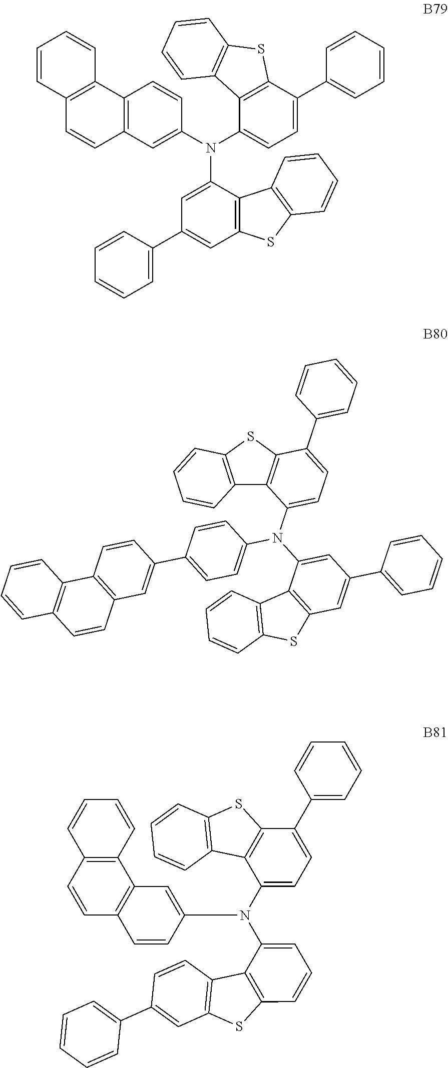 [SMILES: c1ccc(-c2cc(N(c3ccc4c(ccc5ccccc54)c3)c3ccc(-c4ccccc4)c4sc5ccccc5c34)c3c(c2)sc2ccccc23)cc1 ; c1ccc(-c2cc(N(c3ccc(-c4ccc5c(ccc6ccccc65)c4)cc3)c3ccc(-c4ccccc4)c4sc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5sc6ccccc6c45)c23)cc1]

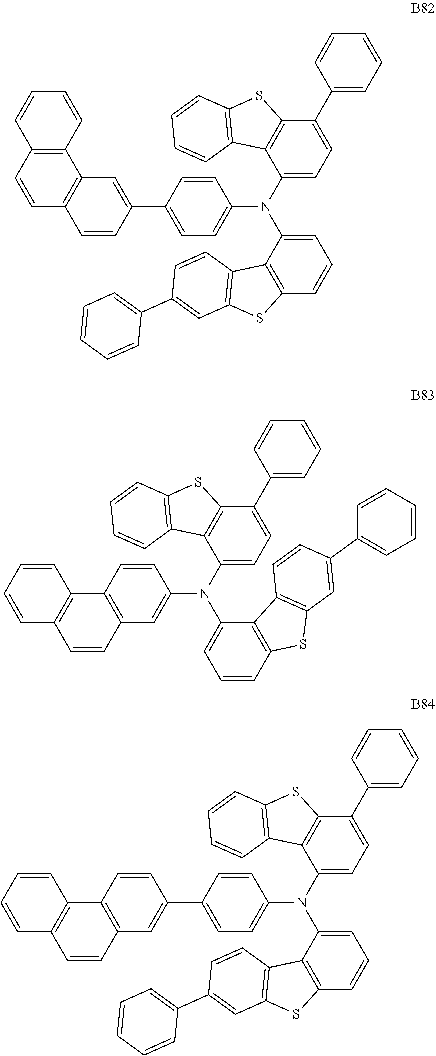 [SMILES: c1ccc(-c2ccc3c(c2)sc2cccc(N(c4ccc(-c5ccc6ccc7ccccc7c6c5)cc4)c4ccc(-c5ccccc5)c5sc6ccccc6c45)c23)cc1 ; c1ccc(-c2ccc3c(c2)sc2cccc(N(c4ccc5c(ccc6ccccc65)c4)c4ccc(-c5ccccc5)c5sc6ccccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5sc6ccccc6c45)c23)cc1]

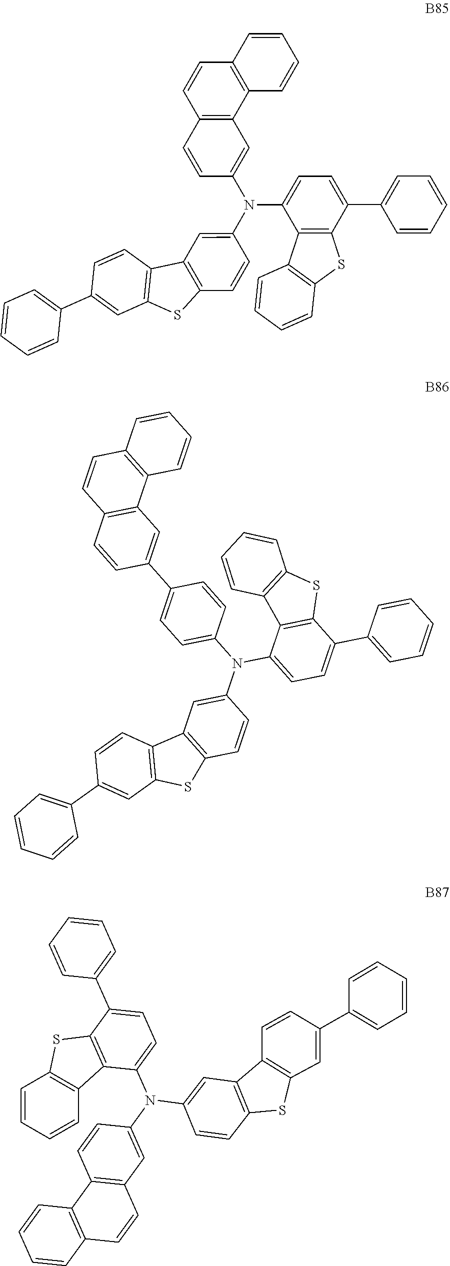 [SMILES: c1ccc(-c2ccc3c(c2)sc2ccc(N(c4ccc5ccc6ccccc6c5c4)c4ccc(-c5ccccc5)c5sc6ccccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5sc6ccccc6c45)cc23)cc1 ; c1ccc(-c2ccc3c(c2)sc2ccc(N(c4ccc5c(ccc6ccccc65)c4)c4ccc(-c5ccccc5)c5sc6ccccc6c45)cc23)cc1]

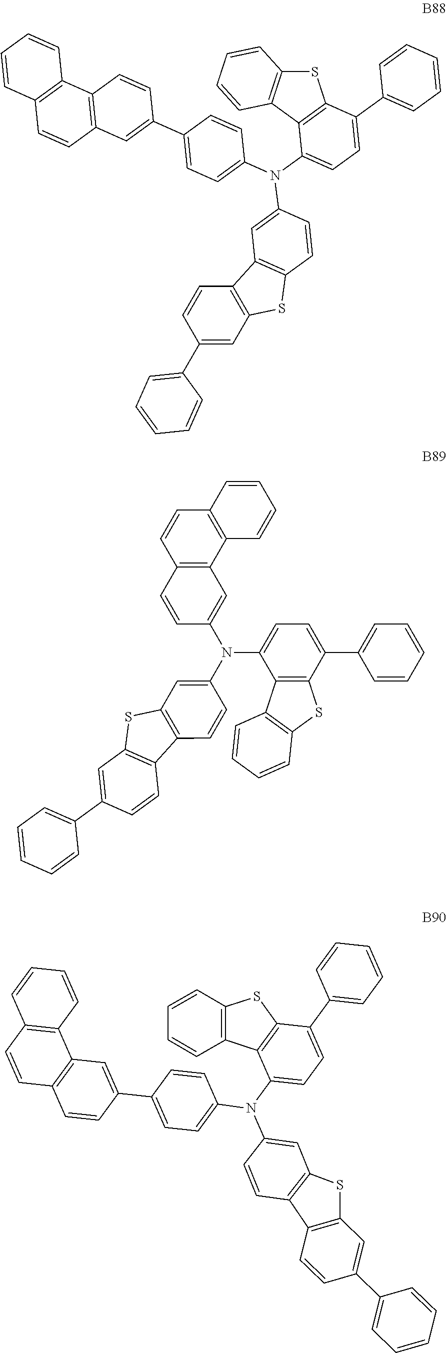 [SMILES: c1ccc(-c2ccc3c(c2)sc2ccc(N(c4ccc(-c5ccc6c(ccc7ccccc76)c5)cc4)c4ccc(-c5ccccc5)c5sc6ccccc6c45)cc23)cc1 ; c1ccc(-c2ccc3c(c2)sc2cc(N(c4ccc5ccc6ccccc6c5c4)c4ccc(-c5ccccc5)c5sc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5sc6ccccc6c45)ccc23)cc1]

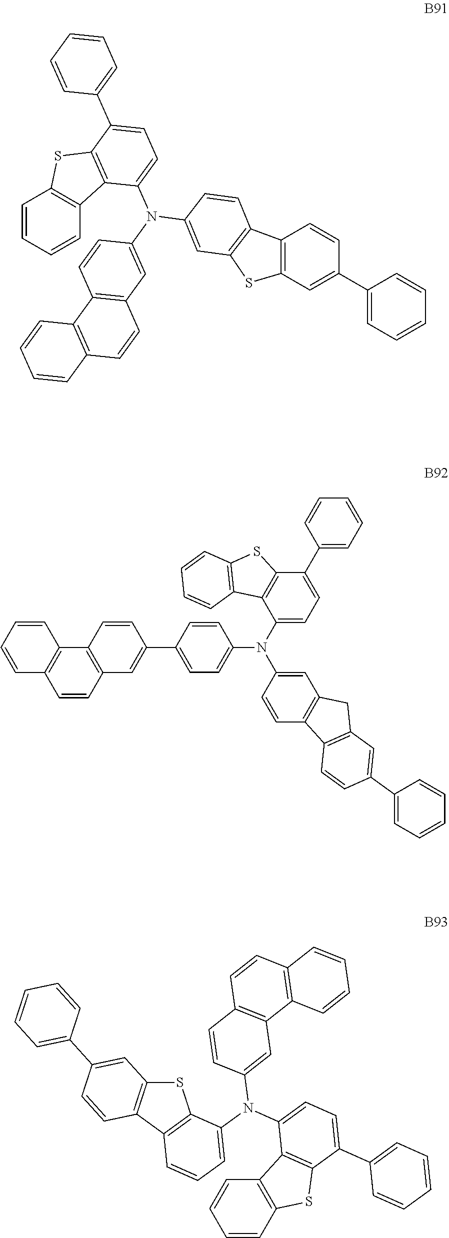 [SMILES: c1ccc(-c2ccc3c(c2)sc2cc(N(c4ccc5c(ccc6ccccc65)c4)c4ccc(-c5ccccc5)c5sc6ccccc6c45)ccc23)cc1 ; c1ccc(-c2ccc3c(c2)Cc2cc(N(c4ccc(-c5ccc6c(ccc7ccccc76)c5)cc4)c4ccc(-c5ccccc5)c5sc6ccccc6c45)ccc2-3)cc1 ; c1ccc(-c2ccc3c(c2)sc2c(N(c4ccc5ccc6ccccc6c5c4)c4ccc(-c5ccccc5)c5sc6ccccc6c45)cccc23)cc1]

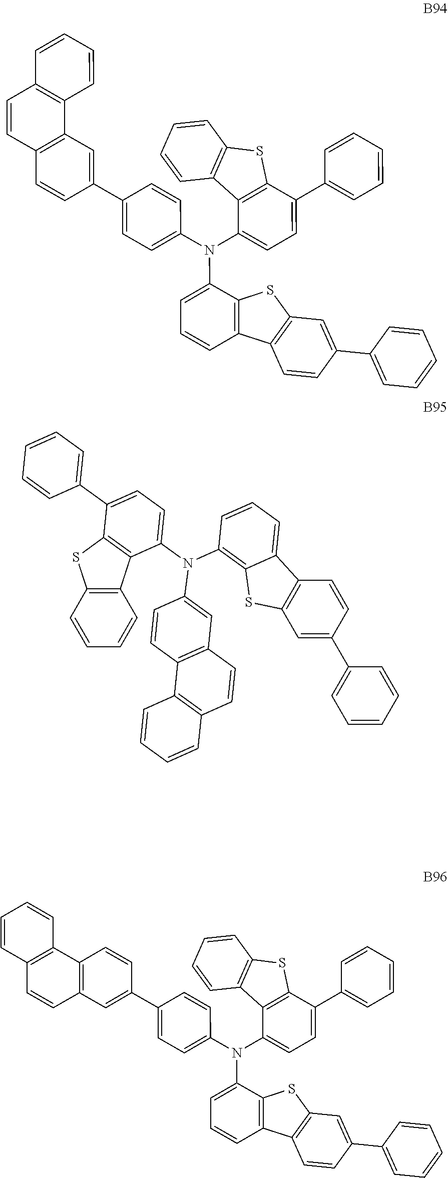 [SMILES: c1ccc(-c2ccc3c(c2)sc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)sc2c(N(c4ccc5c(ccc6ccccc65)c4)c4ccc(-c5ccccc5)c5sc6ccccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5sc6ccccc6c45)cccc23)cc1]

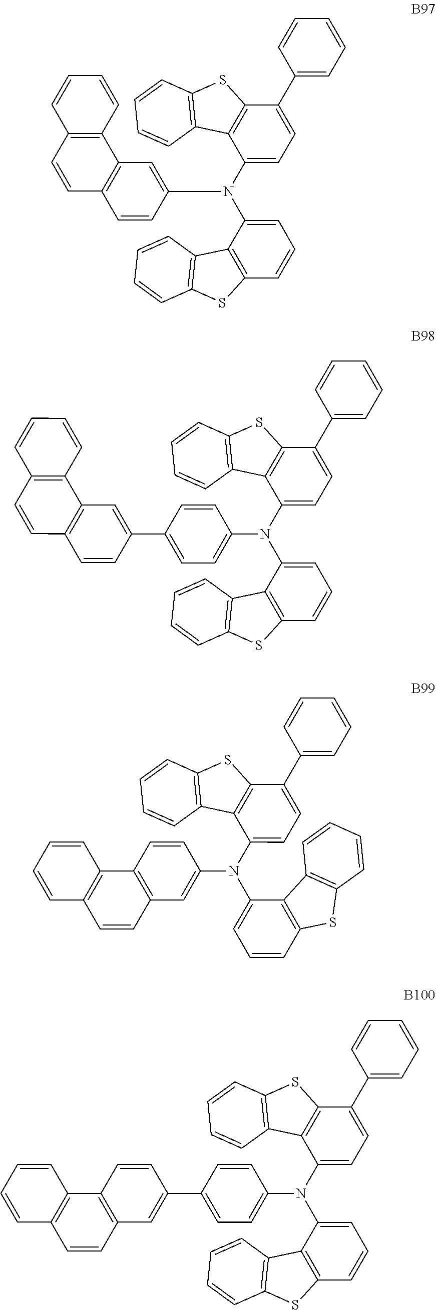 [SMILES: c1ccc(-c2ccc(N(c3ccc4ccc5ccccc5c4c3)c3cccc4sc5ccccc5c34)c3c2sc2ccccc23)cc1 ; c1ccc(-c2ccc(N(c3ccc(-c4ccc5ccc6ccccc6c5c4)cc3)c3cccc4sc5ccccc5c34)c3c2sc2ccccc23)cc1 ; c1ccc(-c2ccc(N(c3ccc4c(ccc5ccccc54)c3)c3cccc4sc5ccccc5c34)c3c2sc2ccccc23)cc1 ; c1ccc(-c2ccc(N(c3ccc(-c4ccc5c(ccc6ccccc65)c4)cc3)c3cccc4sc5ccccc5c34)c3c2sc2ccccc23)cc1]

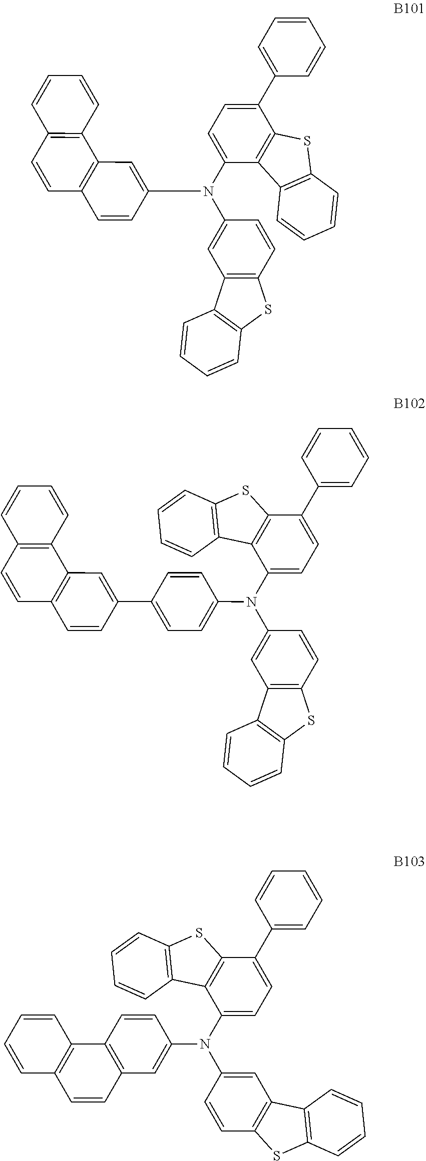 [SMILES: c1ccc(-c2ccc(N(c3ccc4ccc5ccccc5c4c3)c3ccc4sc5ccccc5c4c3)c3c2sc2ccccc23)cc1 ; c1ccc(-c2ccc(N(c3ccc(-c4ccc5ccc6ccccc6c5c4)cc3)c3ccc4sc5ccccc5c4c3)c3c2sc2ccccc23)cc1 ; c1ccc(-c2ccc(N(c3ccc4c(ccc5ccccc54)c3)c3ccc4sc5ccccc5c4c3)c3c2sc2ccccc23)cc1]

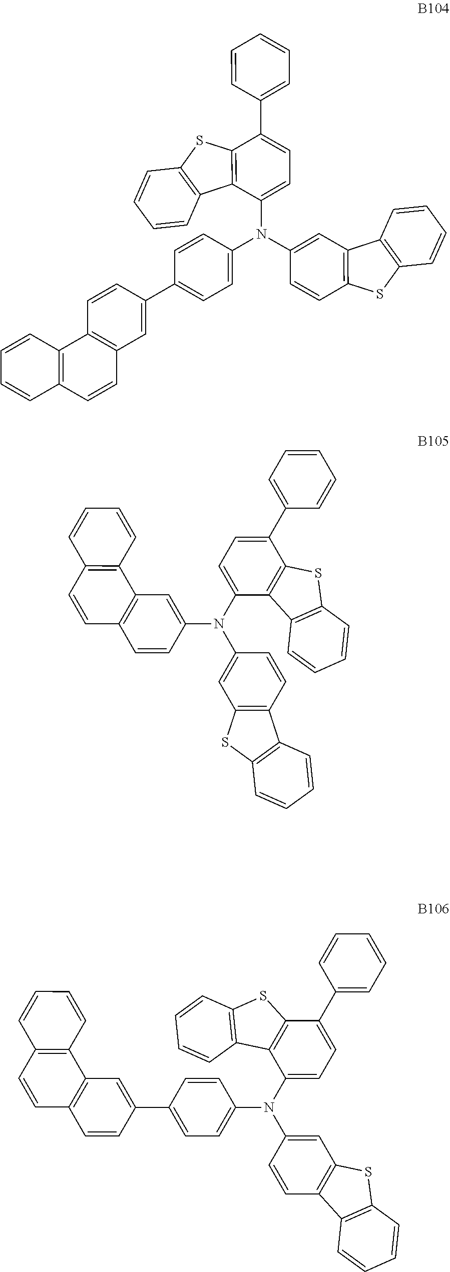 [SMILES: c1ccc(-c2ccc(N(c3ccc(-c4ccc5c(ccc6ccccc65)c4)cc3)c3ccc4sc5ccccc5c4c3)c3c2sc2ccccc23)cc1 ; c1ccc(-c2ccc(N(c3ccc4c(c3)sc3ccccc34)c3ccc4ccc5ccccc5c4c3)c3c2sc2ccccc23)cc1 ; c1ccc(-c2ccc(N(c3ccc(-c4ccc5ccc6ccccc6c5c4)cc3)c3ccc4c(c3)sc3ccccc34)c3c2sc2ccccc23)cc1]

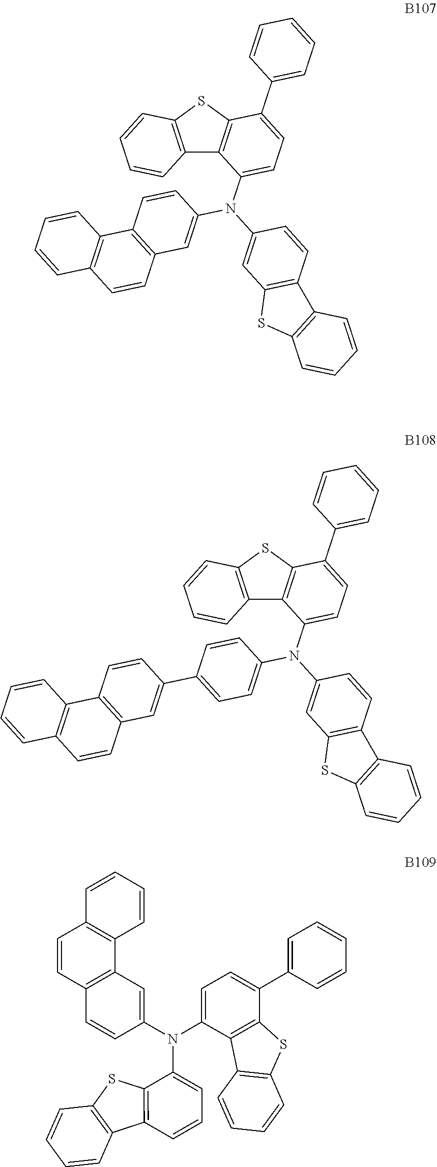 [SMILES: c1ccc(-c2ccc(N(c3ccc4c(ccc5ccccc54)c3)c3ccc4c(c3)sc3ccccc34)c3c2sc2ccccc23)cc1 ; c1ccc(-c2ccc(N(c3ccc(-c4ccc5c(ccc6ccccc65)c4)cc3)c3ccc4c(c3)sc3ccccc34)c3c2sc2ccccc23)cc1 ; c1ccc(-c2ccc(N(c3ccc4ccc5ccccc5c4c3)c3cccc4c3sc3ccccc34)c3c2sc2ccccc23)cc1]

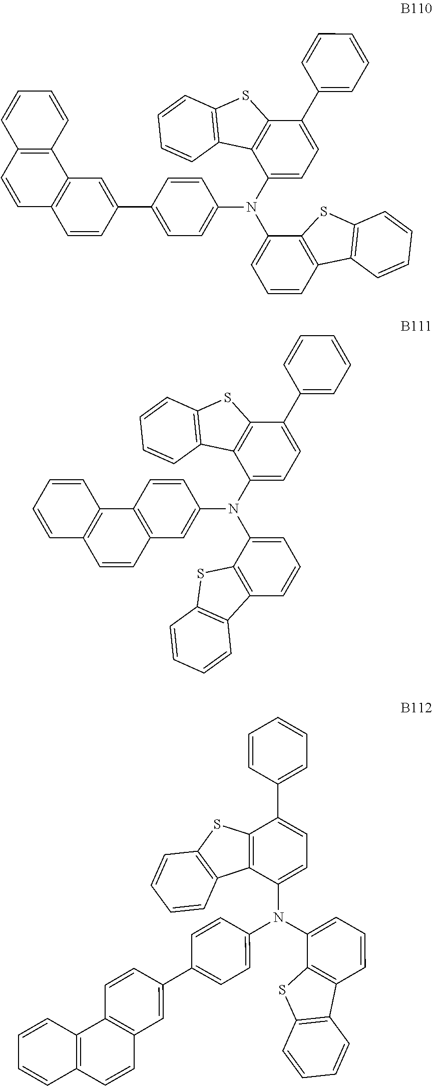

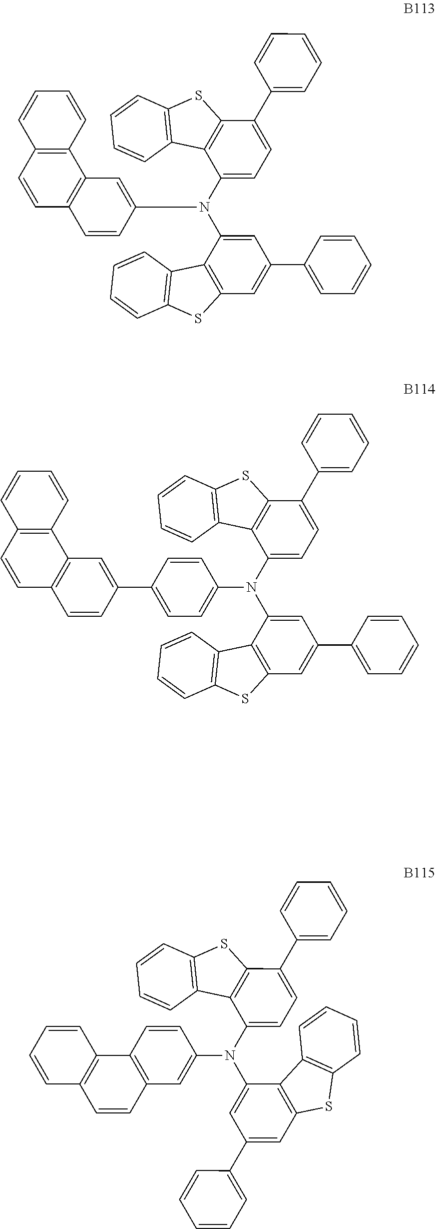 [SMILES: c1ccc(-c2cc(N(c3ccc4ccc5ccccc5c4c3)c3ccc(-c4ccccc4)c4sc5ccccc5c34)c3c(c2)sc2ccccc23)cc1 ; c1ccc(-c2cc(N(c3ccc(-c4ccc5ccc6ccccc6c5c4)cc3)c3ccc(-c4ccccc4)c4sc5ccccc5c34)c3c(c2)sc2ccccc23)cc1 ; c1ccc(-c2cc(N(c3ccc4c(ccc5ccccc54)c3)c3ccc(-c4ccccc4)c4sc5ccccc5c34)c3c(c2)sc2ccccc23)cc1]

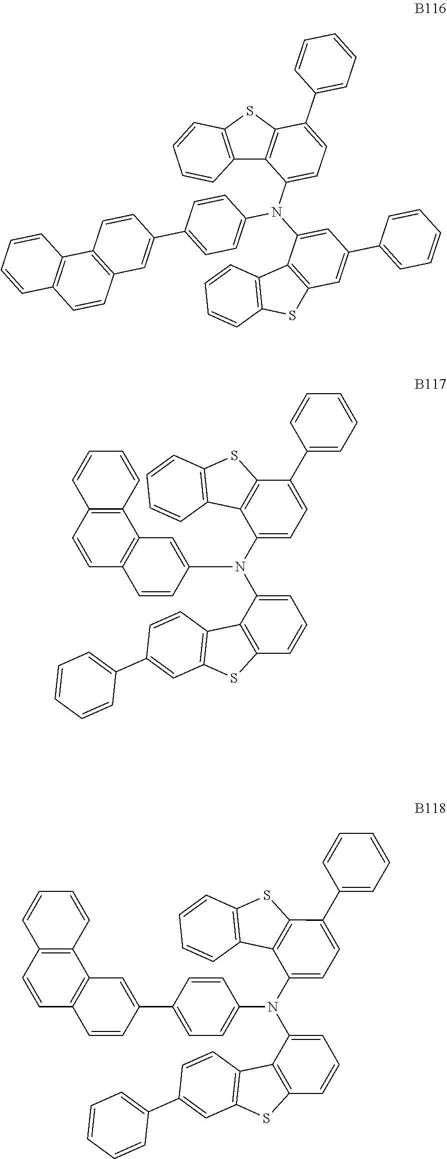 [SMILES: c1ccc(-c2cc(N(c3ccc(-c4ccc5c(ccc6ccccc65)c4)cc3)c3ccc(-c4ccccc4)c4sc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5sc6ccccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5sc6ccccc6c45)c23)cc1]

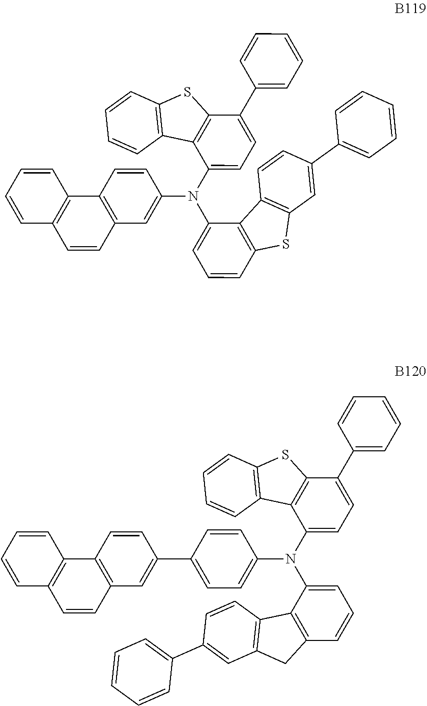 [SMILES: c1ccc(-c2ccc3c(c2)sc2cccc(N(c4ccc5c(ccc6ccccc65)c4)c4ccc(-c5ccccc5)c5sc6ccccc6c45)c23)cc1 ; c1ccc(-c2ccc3c(c2)Cc2cccc(N(c4ccc(-c5ccc6c(ccc7ccccc76)c5)cc4)c4ccc(-c5ccccc5)c5sc6ccccc6c45)c2-3)cc1]

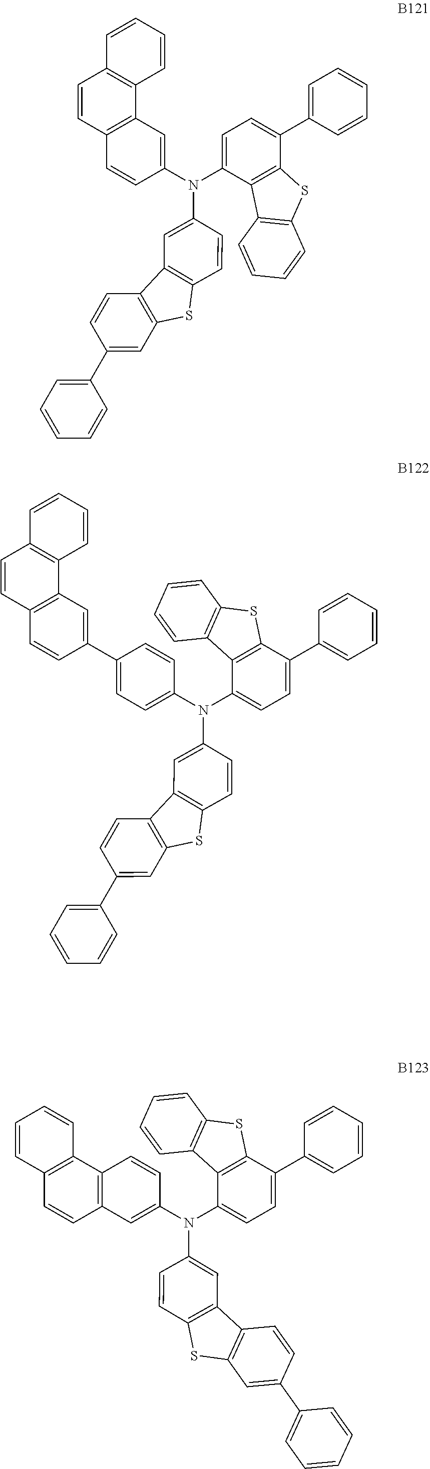 [SMILES: c1ccc(-c2ccc3c(c2)sc2ccc(N(c4ccc5ccc6ccccc6c5c4)c4ccc(-c5ccccc5)c5sc6ccccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5sc6ccccc6c45)cc23)cc1 ; c1ccc(-c2ccc3c(c2)sc2ccc(N(c4ccc5c(ccc6ccccc65)c4)c4ccc(-c5ccccc5)c5sc6ccccc6c45)cc23)cc1]

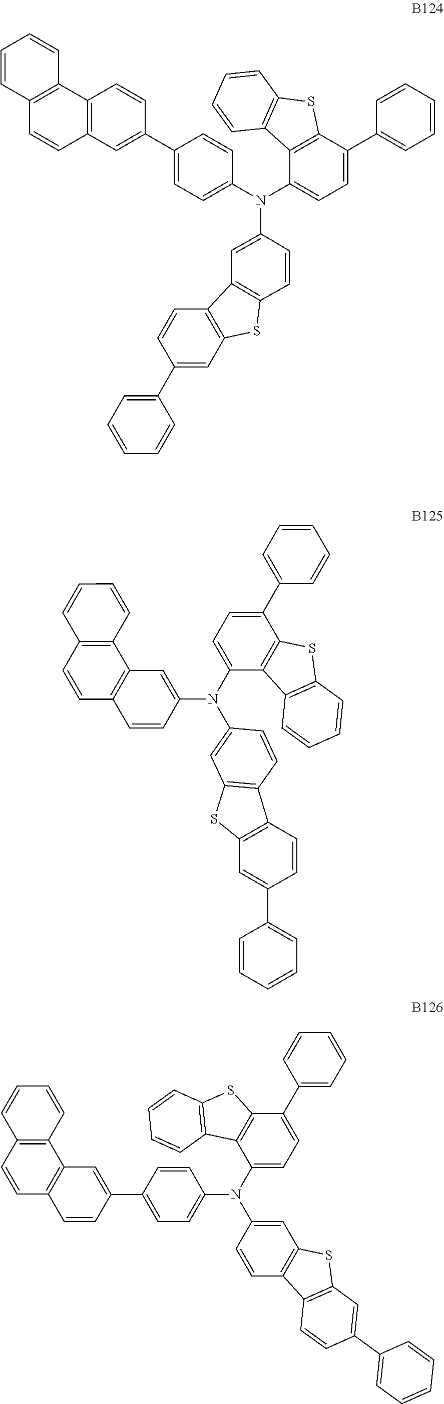 [SMILES: c1ccc(-c2ccc3c(c2)sc2ccc(N(c4ccc(-c5ccc6c(ccc7ccccc76)c5)cc4)c4ccc(-c5ccccc5)c5sc6ccccc6c45)cc23)cc1 ; c1ccc(-c2ccc3c(c2)sc2cc(N(c4ccc5ccc6ccccc6c5c4)c4ccc(-c5ccccc5)c5sc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5sc6ccccc6c45)ccc23)cc1]

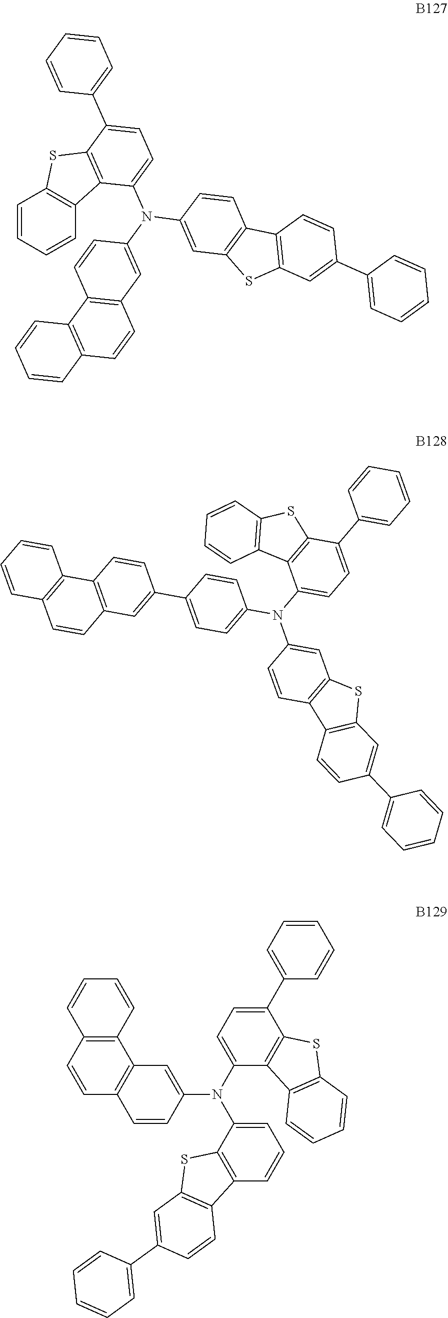 [SMILES: c1ccc(-c2ccc3c(c2)sc2cc(N(c4ccc5c(ccc6ccccc65)c4)c4ccc(-c5ccccc5)c5sc6ccccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5sc6ccccc6c45)ccc23)cc1 ; c1ccc(-c2ccc3c(c2)sc2c(N(c4ccc5ccc6ccccc6c5c4)c4ccc(-c5ccccc5)c5sc6ccccc6c45)cccc23)cc1]

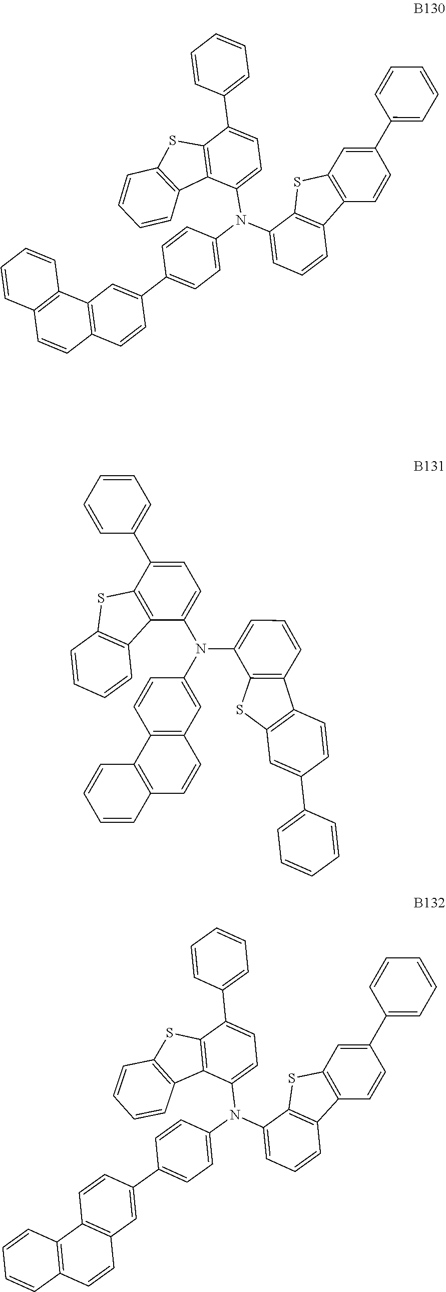 [SMILES: c1ccc(-c2ccc3c(c2)sc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)sc2c(N(c4ccc5c(ccc6ccccc65)c4)c4ccc(-c5ccccc5)c5sc6ccccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5sc6ccccc6c45)cccc23)cc1]

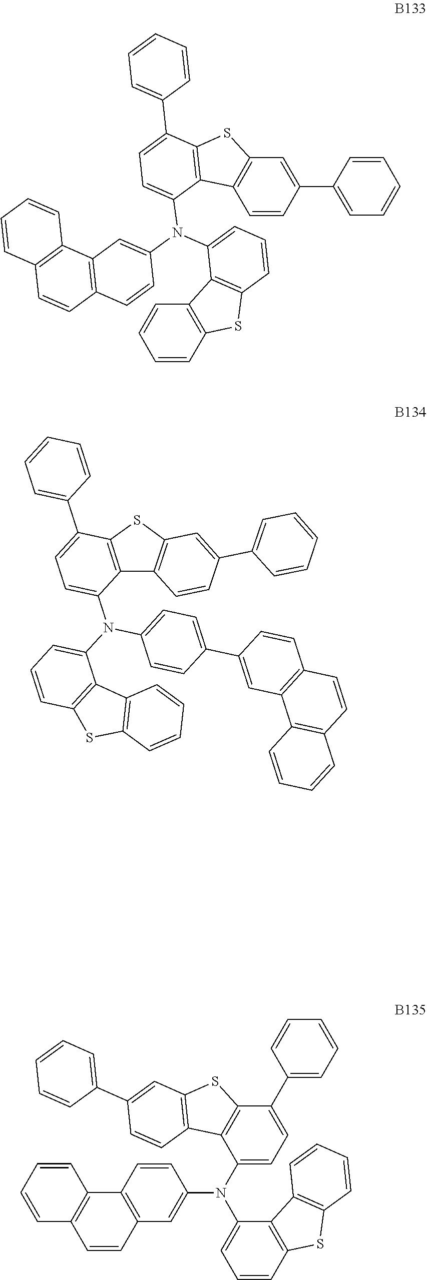 [SMILES: c1ccc(-c2ccc3c(c2)sc2c(-c4ccccc4)ccc(N(c4ccc5ccc6ccccc6c5c4)c4cccc5sc6ccccc6c45)c23)cc1 ; c1ccc(-c2ccc3c(c2)sc2c(-c4ccccc4)ccc(N(c4ccc(-c5ccc6ccc7ccccc7c6c5)cc4)c4cccc5sc6ccccc6c45)c23)cc1 ; c1ccc(-c2ccc3c(c2)sc2c(-c4ccccc4)ccc(N(c4ccc5c(ccc6ccccc65)c4)c4cccc5sc6ccccc6c45)c23)cc1]

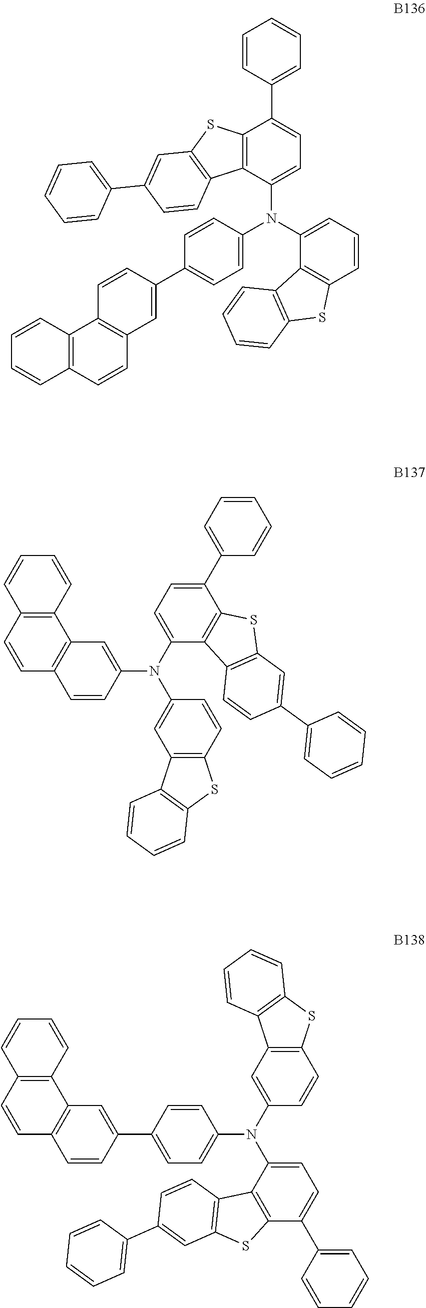 [SMILES: c1ccc(-c2ccc3c(c2)sc2c(-c4ccccc4)ccc(N(c4ccc(-c5ccc6c(ccc7ccccc76)c5)cc4)c4cccc5sc6ccccc6c45)c23)cc1 ; c1ccc(-c2ccc3c(c2)sc2c(-c4ccccc4)ccc(N(c4ccc5ccc6ccccc6c5c4)c4ccc5sc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5sc6ccccc6c5c4)c23)cc1]

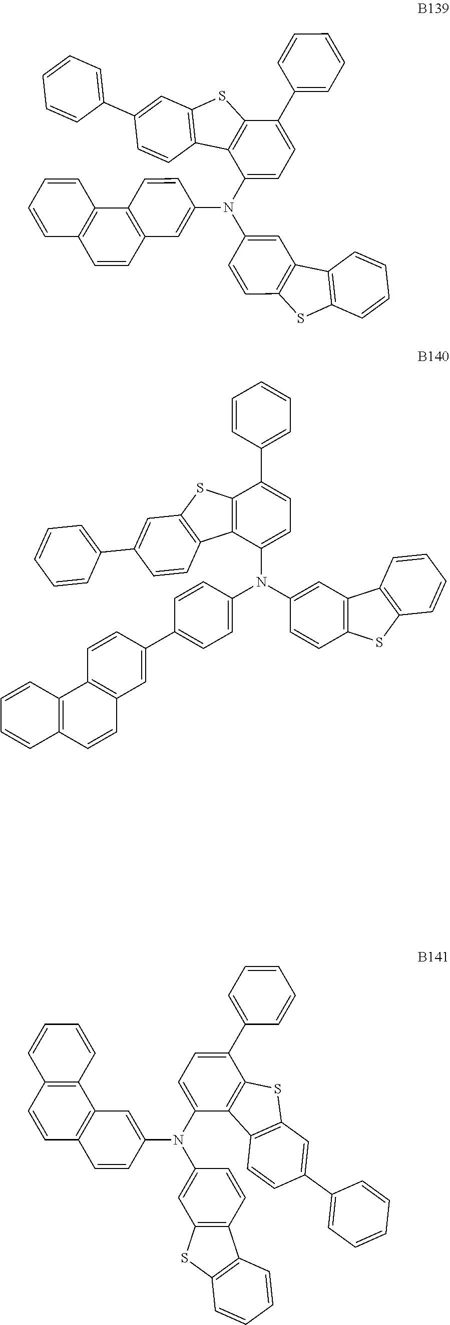 [SMILES: c1ccc(-c2ccc3c(c2)sc2c(-c4ccccc4)ccc(N(c4ccc5c(ccc6ccccc65)c4)c4ccc5sc6ccccc6c5c4)c23)cc1 ; c1ccc(-c2ccc3c(c2)sc2c(-c4ccccc4)ccc(N(c4ccc(-c5ccc6c(ccc7ccccc76)c5)cc4)c4ccc5sc6ccccc6c5c4)c23)cc1 ; c1ccc(-c2ccc3c(c2)sc2c(-c4ccccc4)ccc(N(c4ccc5c(c4)sc4ccccc45)c4ccc5ccc6ccccc6c5c4)c23)cc1]

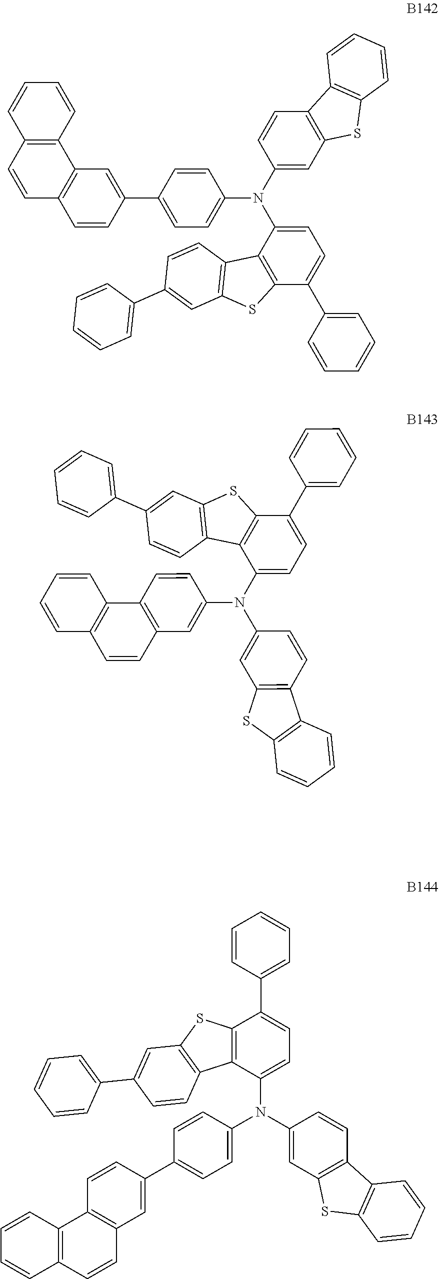 [SMILES: c1ccc(-c2ccc3c(c2)sc2c(-c4ccccc4)ccc(N(c4ccc(-c5ccc6ccc7ccccc7c6c5)cc4)c4ccc5c(c4)sc4ccccc45)c23)cc1 ; c1ccc(-c2ccc3c(c2)sc2c(-c4ccccc4)ccc(N(c4ccc5c(ccc6ccccc65)c4)c4ccc5c(c4)sc4ccccc45)c23)cc1 ; c1ccc(-c2ccc3c(c2)sc2c(-c4ccccc4)ccc(N(c4ccc(-c5ccc6c(ccc7ccccc76)c5)cc4)c4ccc5c(c4)sc4ccccc45)c23)cc1]

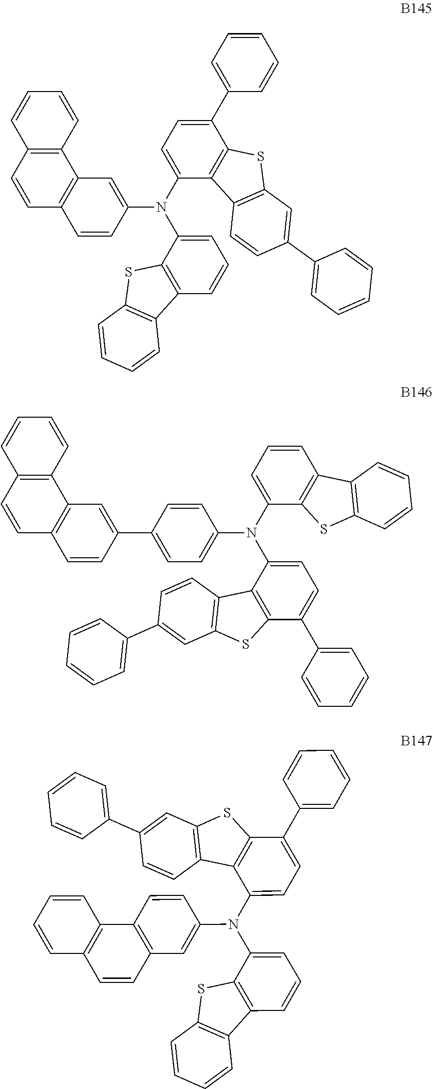

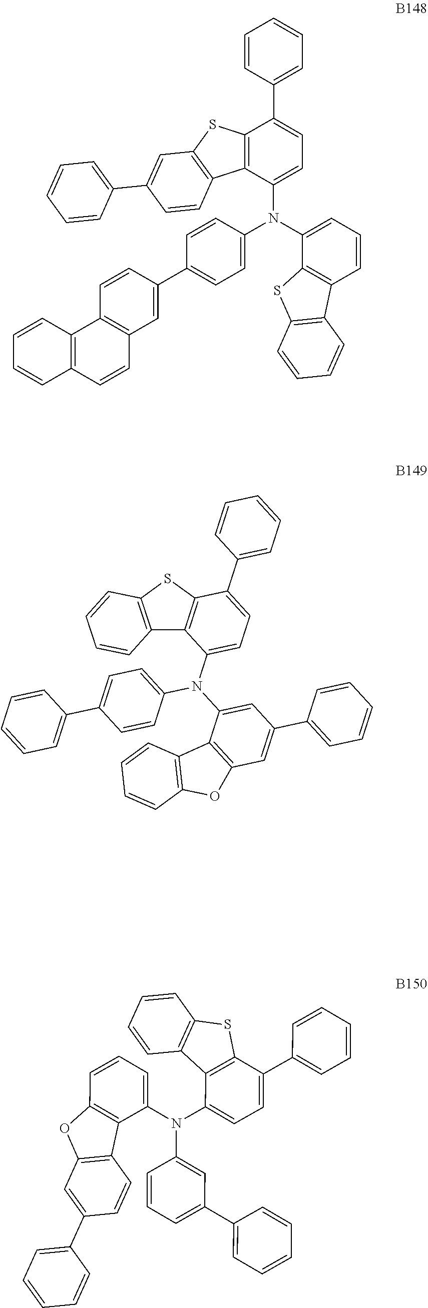 [SMILES: c1ccc(-c2ccc3c(c2)sc2c(-c4ccccc4)ccc(N(c4ccc(-c5ccc6c(ccc7ccccc76)c5)cc4)c4cccc5c4sc4ccccc45)c23)cc1 ; c1ccc(-c2ccc(N(c3cc(-c4ccccc4)cc4oc5ccccc5c34)c3ccc(-c4ccccc4)c4sc5ccccc5c34)cc2)cc1 ; c1ccc(-c2cccc(N(c3cccc4oc5cc(-c6ccccc6)ccc5c34)c3ccc(-c4ccccc4)c4sc5ccccc5c34)c2)cc1]

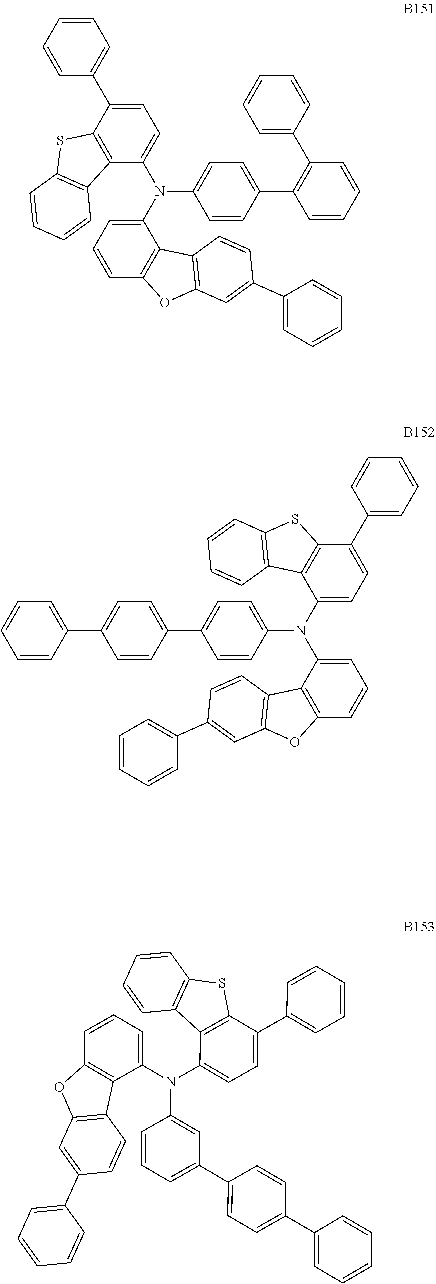 [SMILES: c1ccc(-c2ccc3c(c2)oc2cccc(N(c4ccc(-c5ccccc5-c5ccccc5)cc4)c4ccc(-c5ccccc5)c5sc6ccccc6c45)c23)cc1 ; c1ccc(-c2ccc(-c3ccc(N(c4cccc5oc6cc(-c7ccccc7)ccc6c45)c4ccc(-c5ccccc5)c5sc6ccccc6c45)cc3)cc2)cc1 ; c1ccc(-c2ccc(-c3cccc(N(c4cccc5oc6cc(-c7ccccc7)ccc6c45)c4ccc(-c5ccccc5)c5sc6ccccc6c45)c3)cc2)cc1]

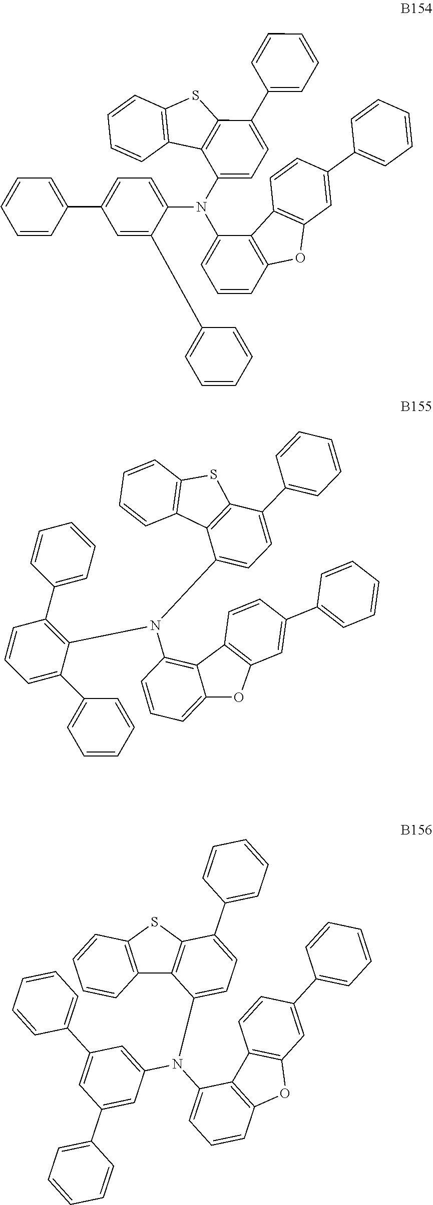 [SMILES: c1ccc(-c2ccc(N(c3cccc4oc5cc(-c6ccccc6)ccc5c34)c3ccc(-c4ccccc4)c4sc5ccccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5sc6ccccc6c45)c23)cc1 ; c1ccc(-c2cc(-c3ccccc3)cc(N(c3cccc4oc5cc(-c6ccccc6)ccc5c34)c3ccc(-c4ccccc4)c4sc5ccccc5c34)c2)cc1]

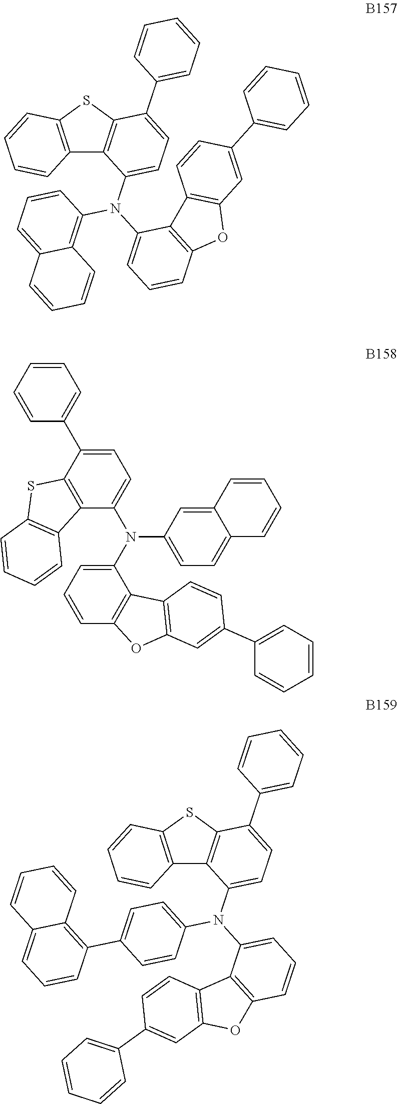

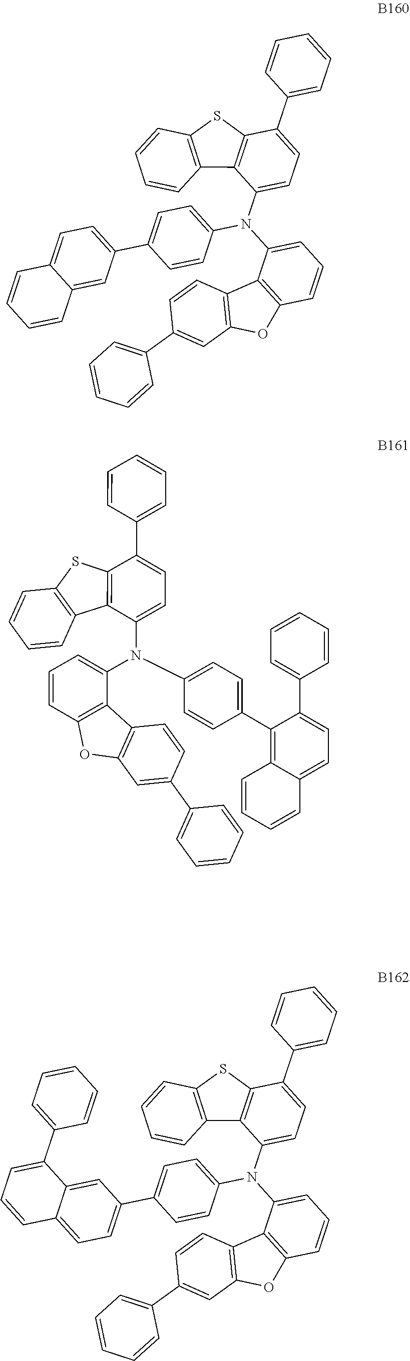 [SMILES: c1ccc(-c2ccc3c(c2)oc2cccc(N(c4ccc(-c5ccc6ccccc6c5)cc4)c4ccc(-c5ccccc5)c5sc6ccccc6c45)c23)cc1 ; c1ccc(-c2ccc3c(c2)oc2cccc(N(c4ccc(-c5c(-c6ccccc6)ccc6ccccc56)cc4)c4ccc(-c5ccccc5)c5sc6ccccc6c45)c23)cc1 ; c1ccc(-c2ccc3c(c2)oc2cccc(N(c4ccc(-c5ccc6cccc(-c7ccccc7)c6c5)cc4)c4ccc(-c5ccccc5)c5sc6ccccc6c45)c23)cc1]

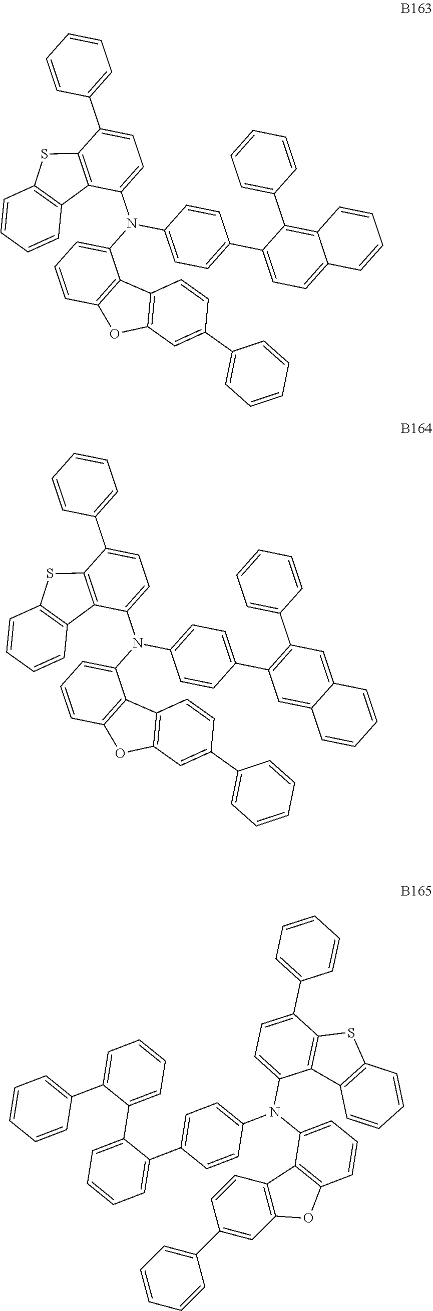 [SMILES: c1ccc(-c2ccc3c(c2)oc2cccc(N(c4ccc(-c5ccc6ccccc6c5-c5ccccc5)cc4)c4ccc(-c5ccccc5)c5sc6ccccc6c45)c23)cc1 ; c1ccc(-c2ccc3c(c2)oc2cccc(N(c4ccc(-c5cc6ccccc6cc5-c5ccccc5)cc4)c4ccc(-c5ccccc5)c5sc6ccccc6c45)c23)cc1 ; c1ccc(-c2ccc3c(c2)oc2cccc(N(c4ccc(-c5ccccc5-c5ccccc5-c5ccccc5)cc4)c4ccc(-c5ccccc5)c5sc6ccccc6c45)c23)cc1]

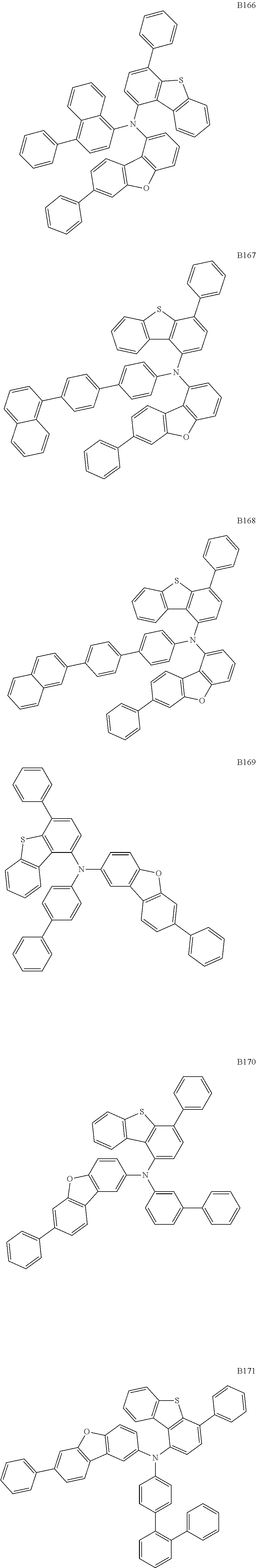

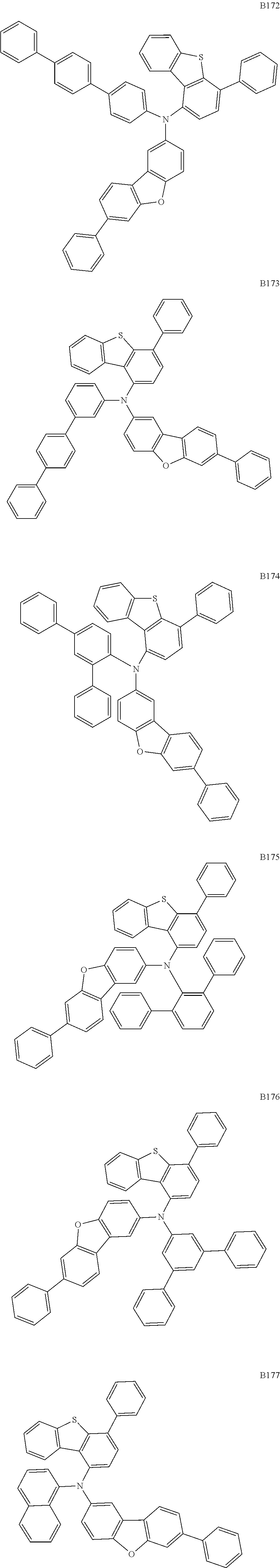

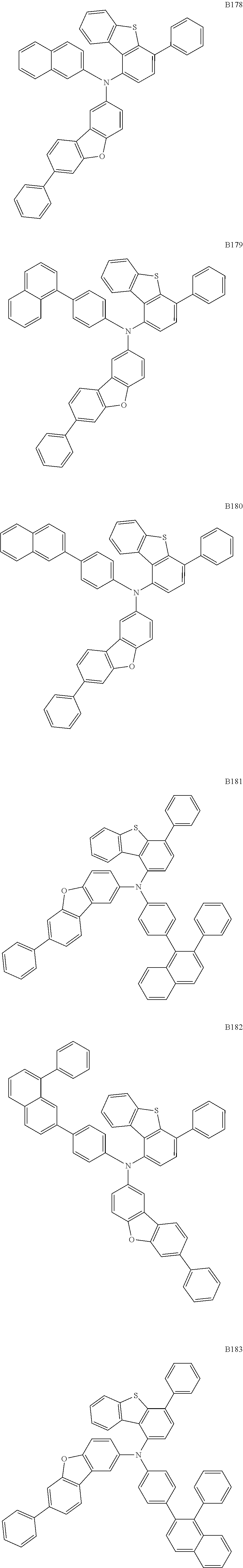

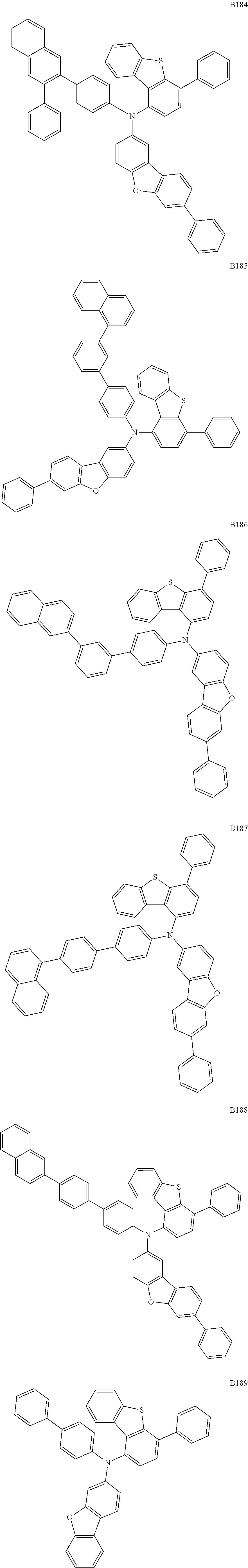

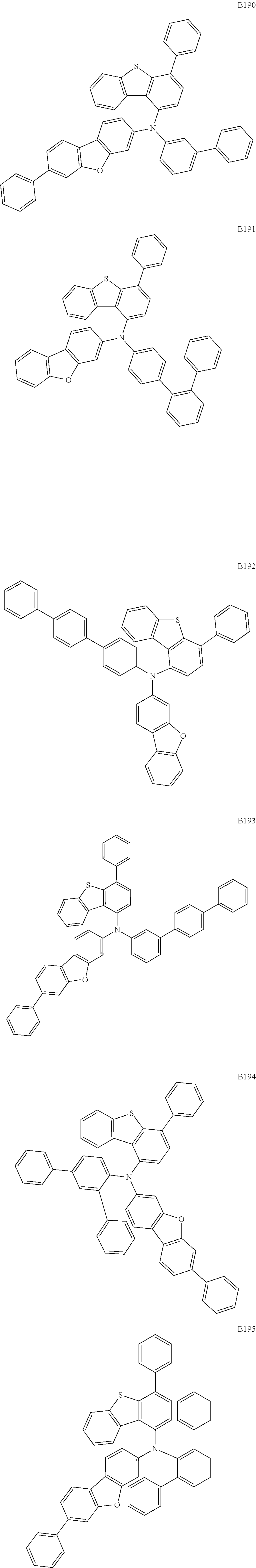

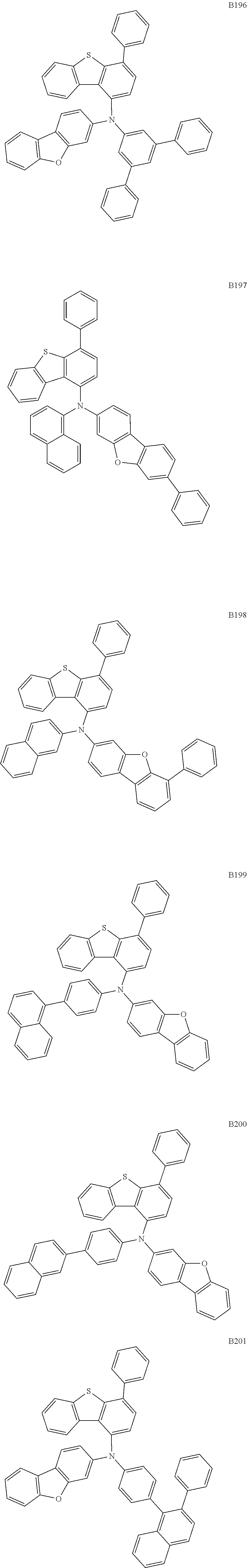

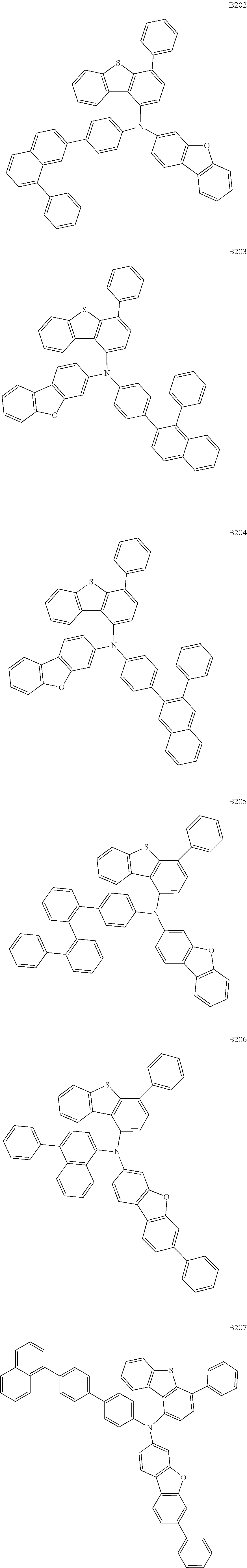

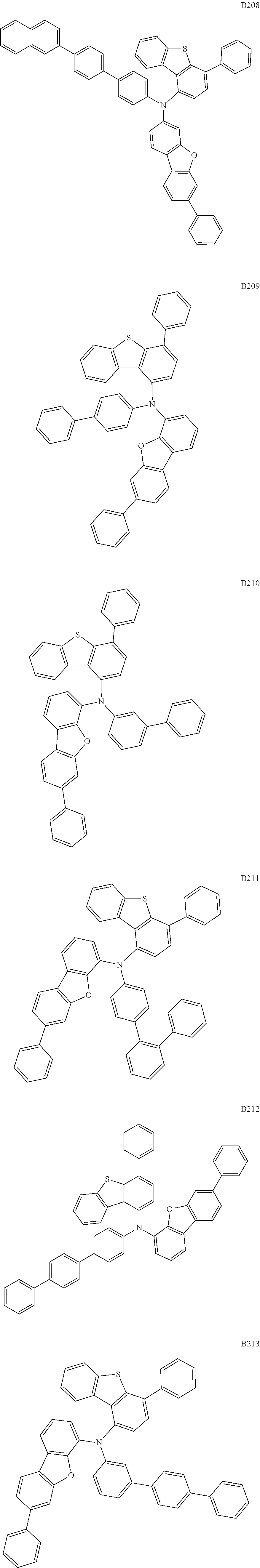

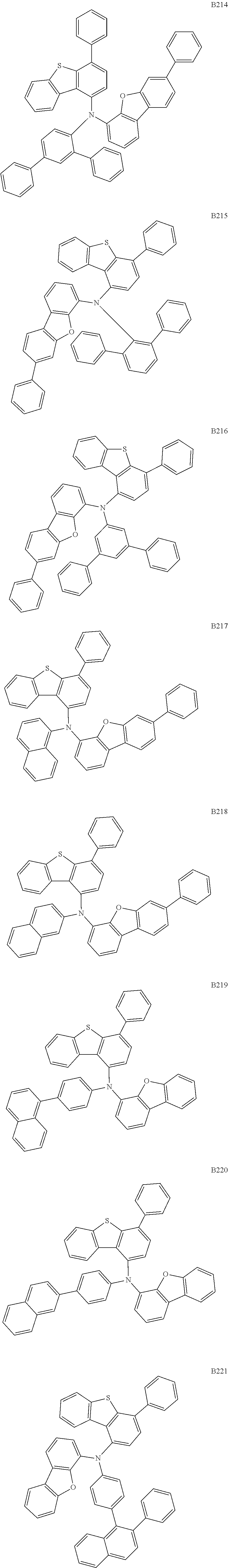

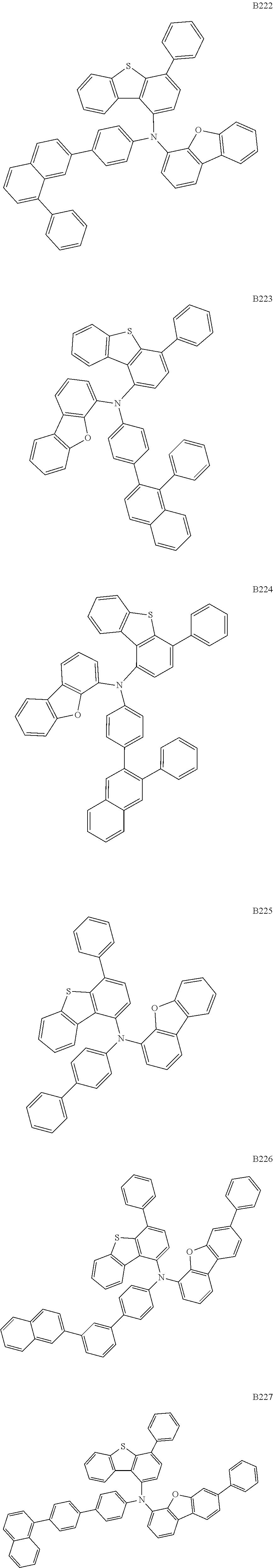

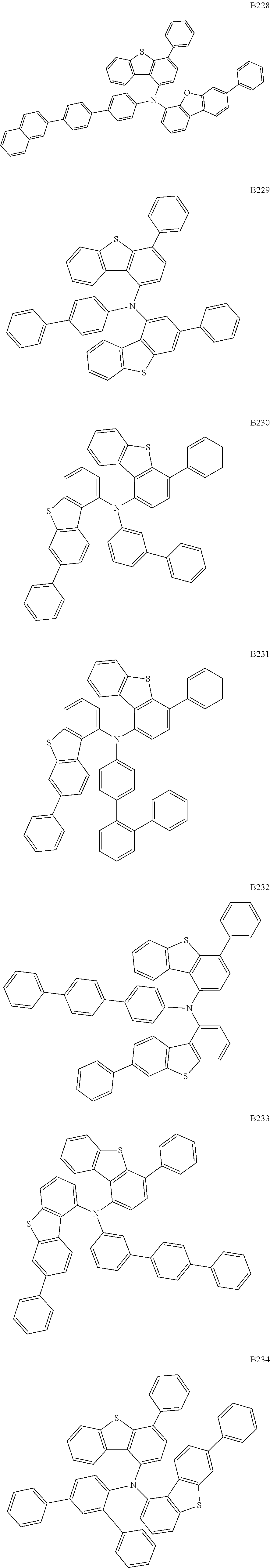

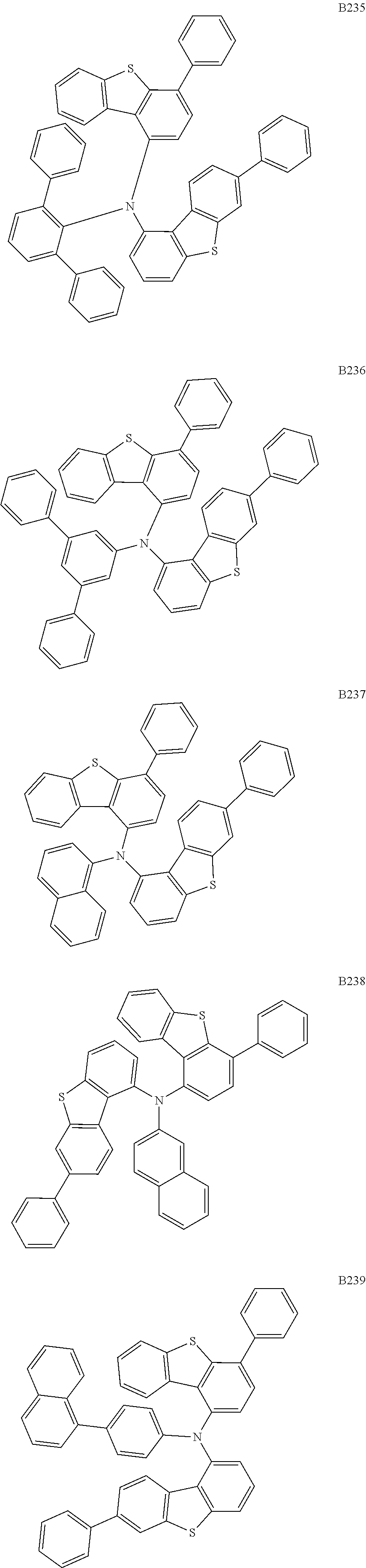 [SMILES: c1ccc(-c2ccc3c(c2)sc2cccc(N(c4c(-c5ccccc5)cccc4-c4ccccc4)c4ccc(-c5ccccc5)c5sc6ccccc6c45)c23)cc1 ; c1ccc(-c2cc(-c3ccccc3)cc(N(c3cccc4sc5cc(-c6ccccc6)ccc5c34)c3ccc(-c4ccccc4)c4sc5ccccc5c34)c2)cc1 ; c1ccc(-c2ccc3c(c2)sc2cccc(N(c4cccc5ccccc45)c4ccc(-c5ccccc5)c5sc6ccccc6c45)c23)cc1 ; c1ccc(-c2ccc3c(c2)sc2cccc(N(c4ccc5ccccc5c4)c4ccc(-c5ccccc5)c5sc6ccccc6c45)c23)cc1 ; c1ccc(-c2ccc3c(c2)sc2cccc(N(c4ccc(-c5cccc6ccccc56)cc4)c4ccc(-c5ccccc5)c5sc6ccccc6c45)c23)cc1]

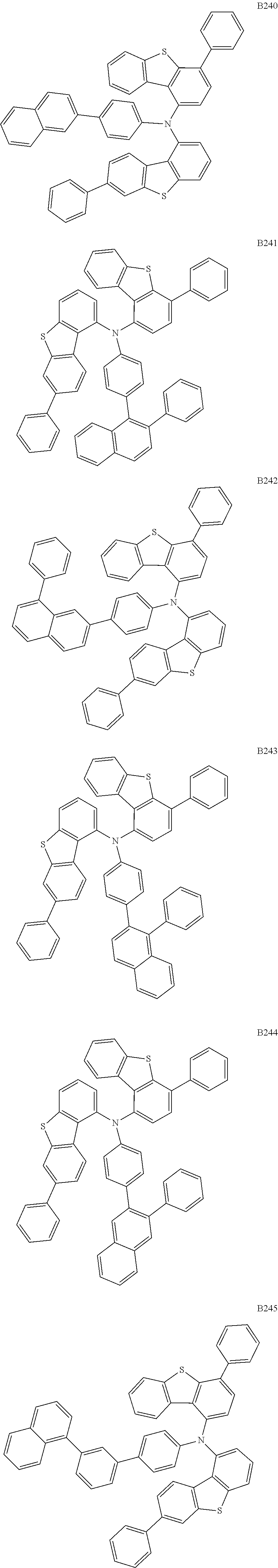

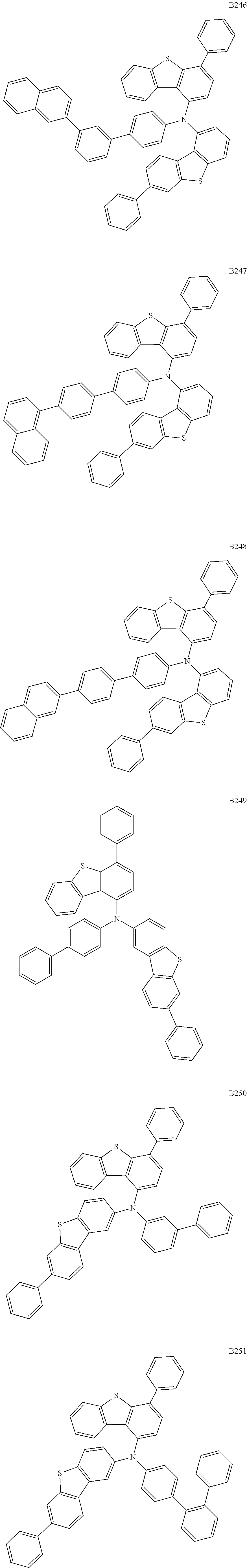

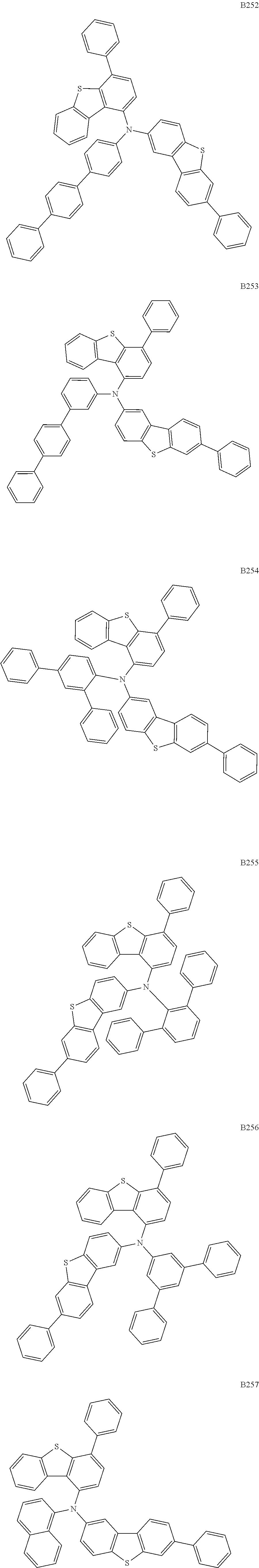

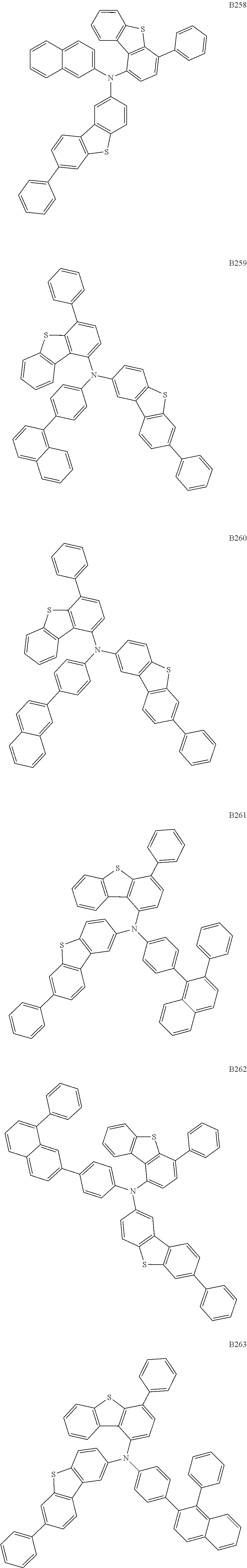

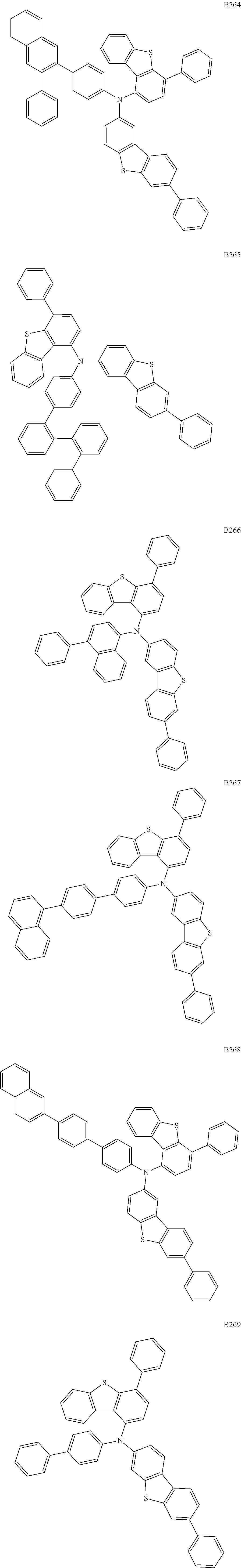

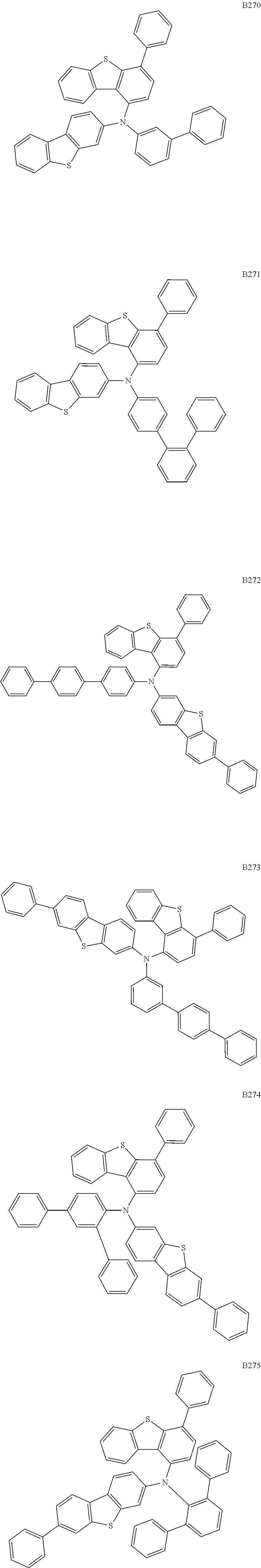

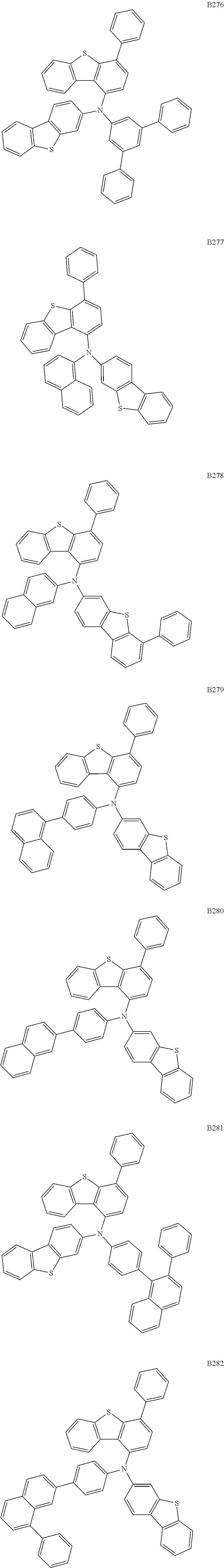

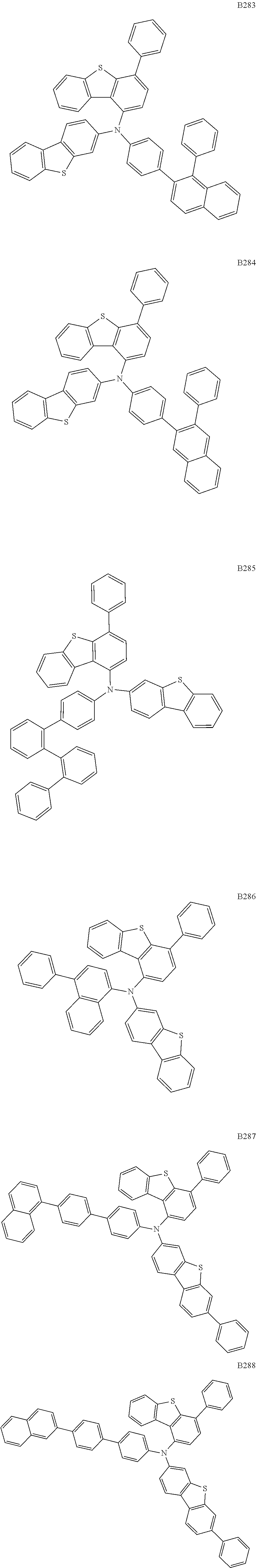

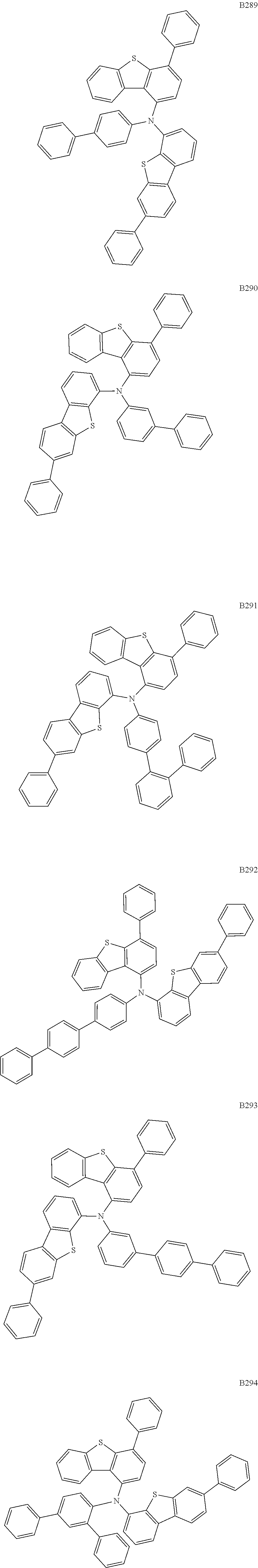

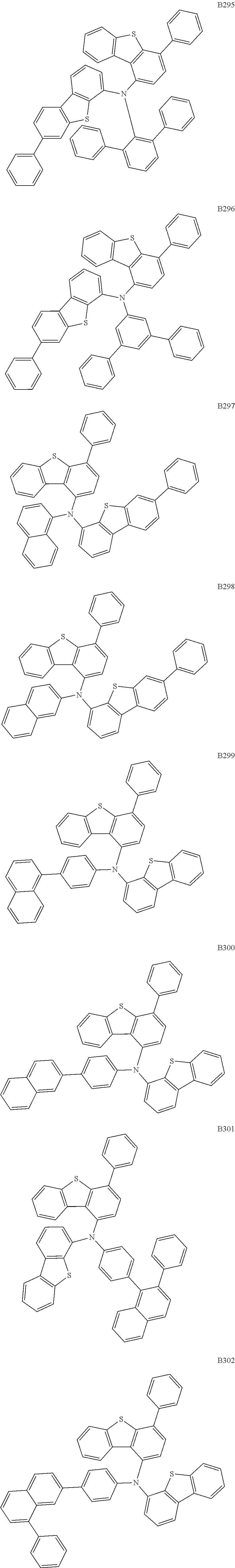

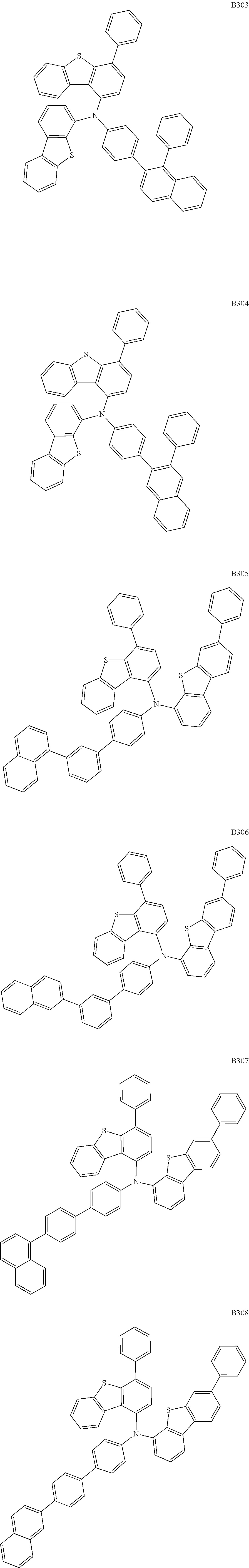

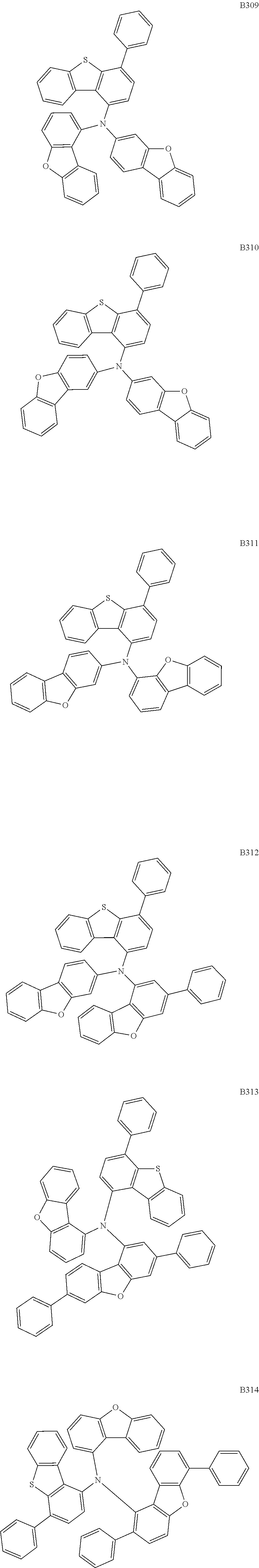

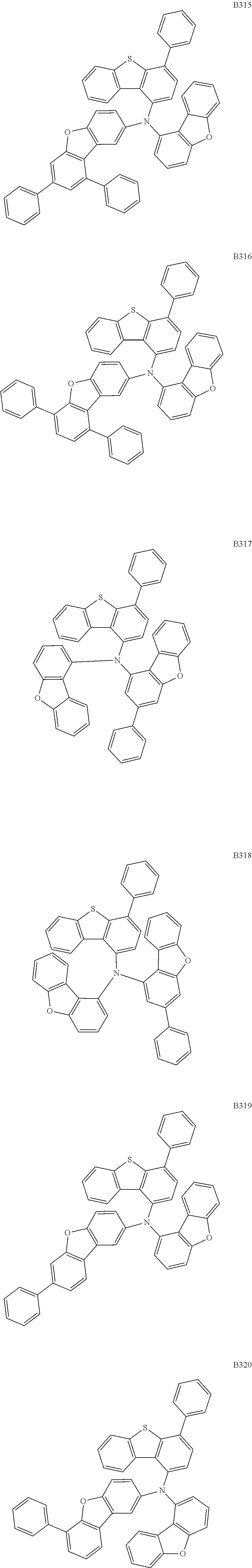

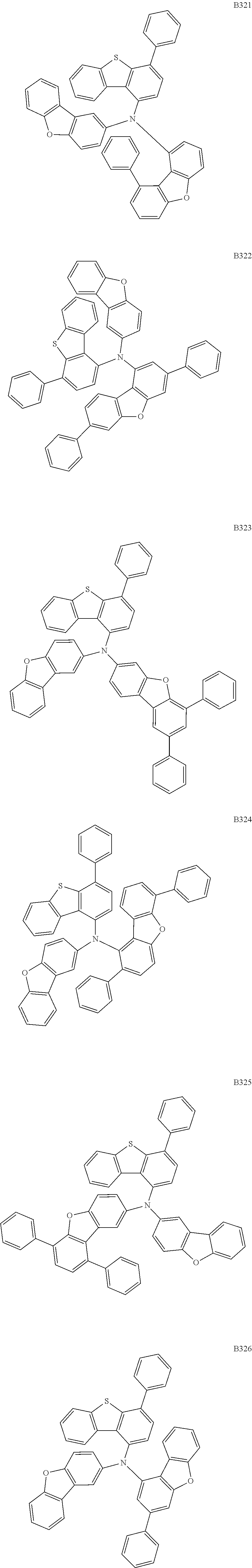

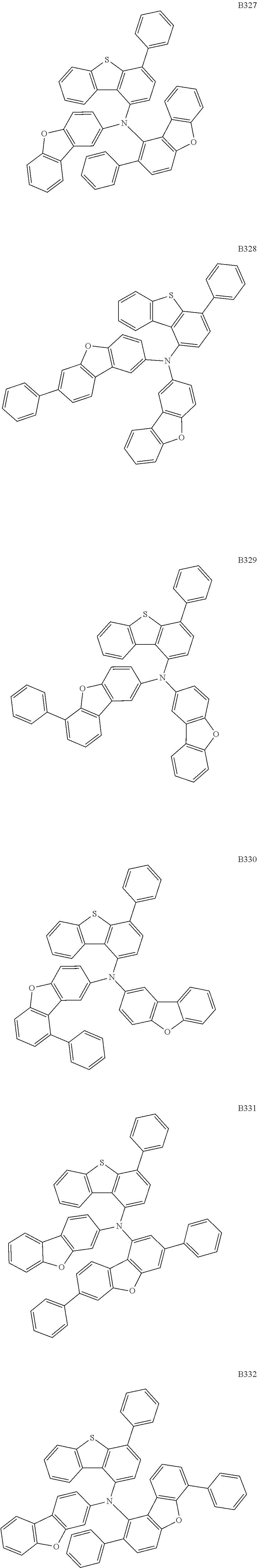

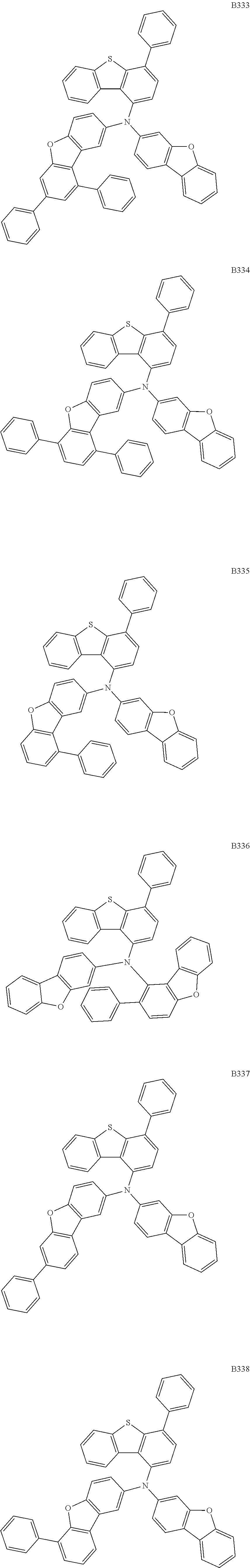

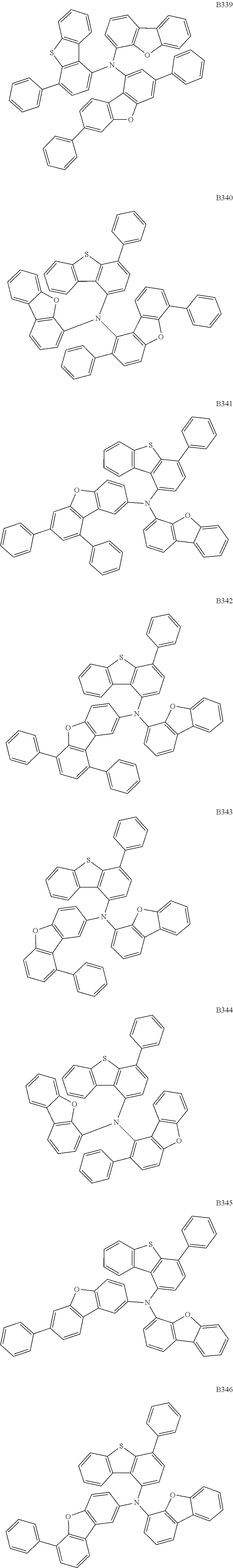

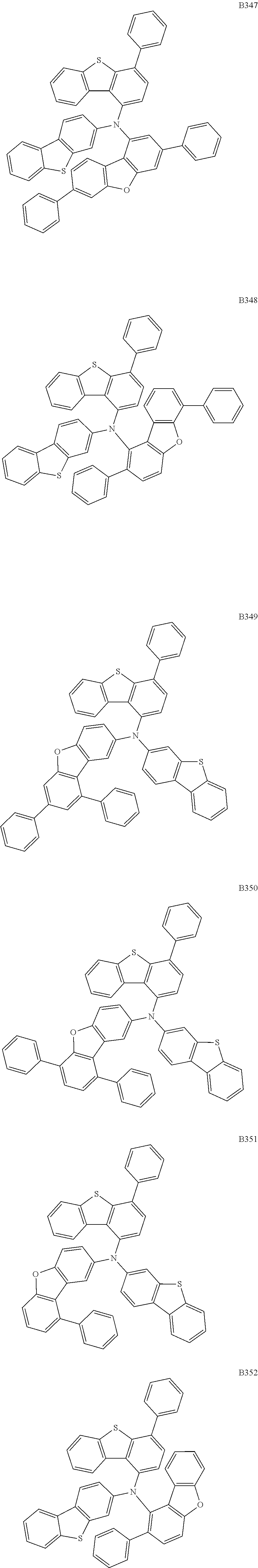

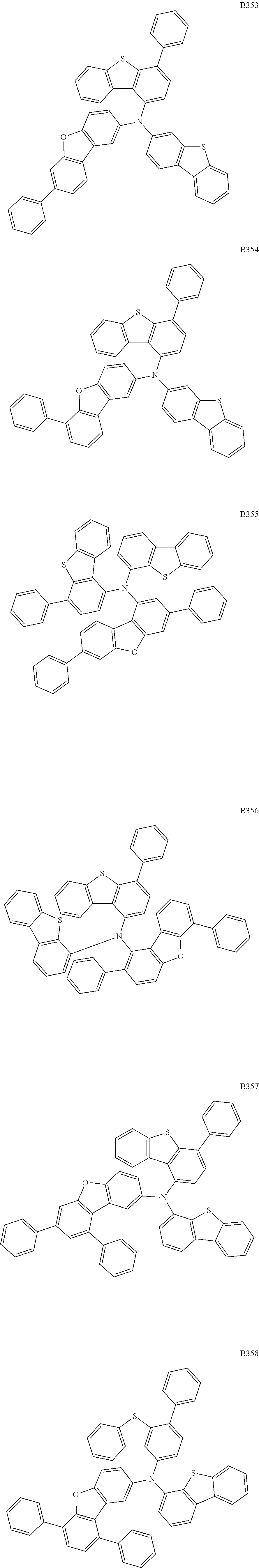

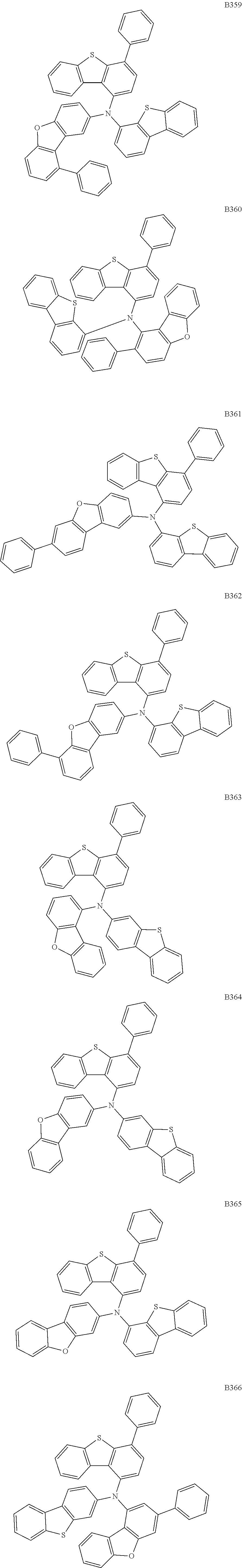

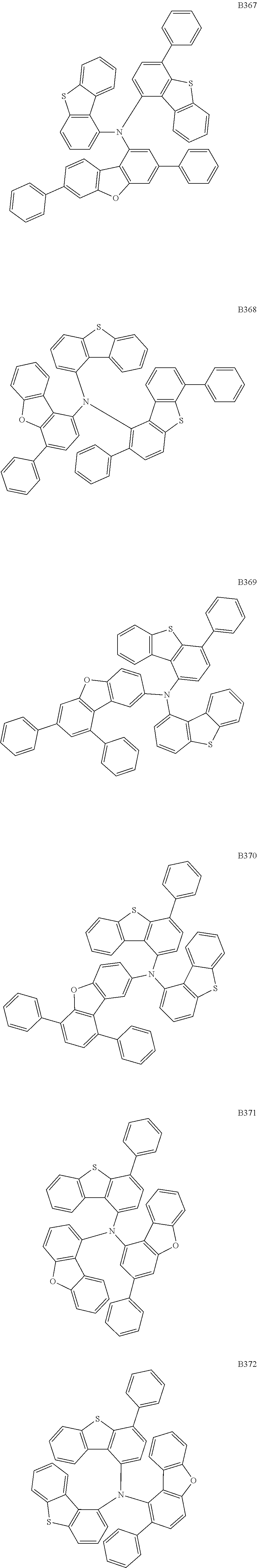

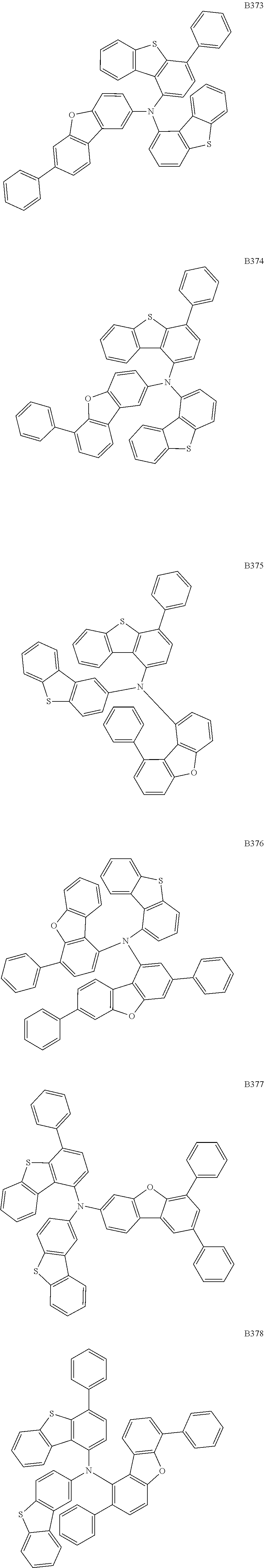

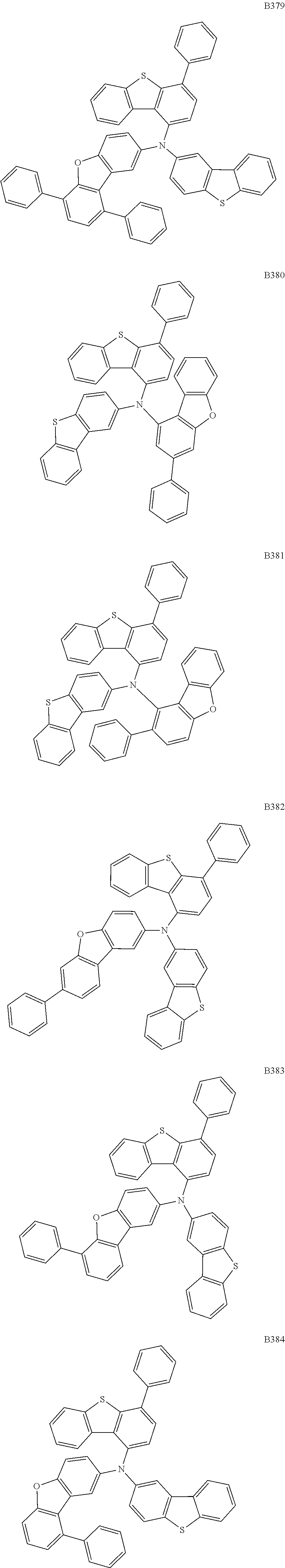

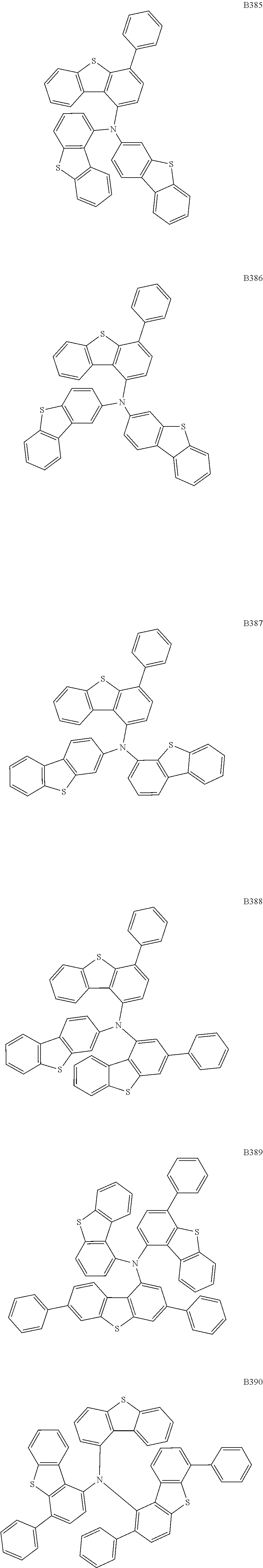

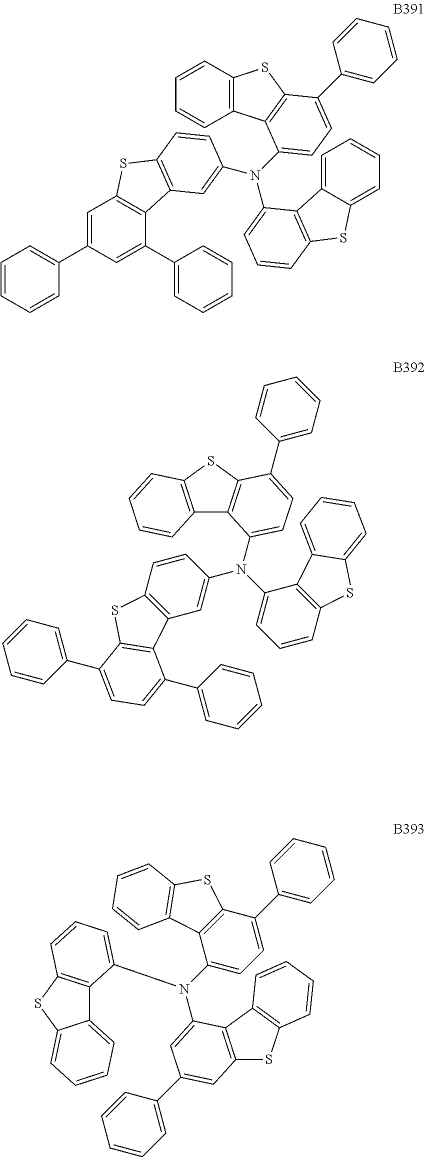 [SMILES: c1ccc(-c2cc(-c3ccccc3)c3c(c2)sc2ccc(N(c4cccc5sc6ccccc6c45)c4ccc(-c5ccccc5)c5sc6ccccc6c45)cc23)cc1 ; c1ccc(-c2ccc(-c3ccccc3)c3c2sc2ccc(N(c4cccc5sc6ccccc6c45)c4ccc(-c5ccccc5)c5sc6ccccc6c45)cc23)cc1 ; c1ccc(-c2cc(N(c3cccc4sc5ccccc5c34)c3ccc(-c4ccccc4)c4sc5ccccc5c34)c3c(c2)sc2ccccc23)cc1]

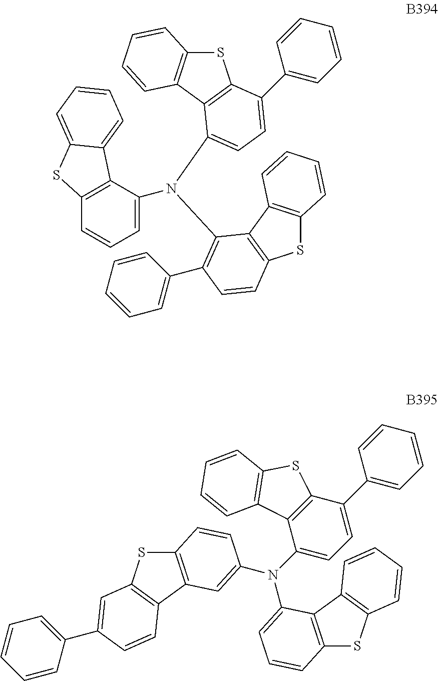 [SMILES: c1ccc(-c2ccc3sc4ccccc4c3c2N(c2cccc3sc4ccccc4c23)c2ccc(-c3ccccc3)c3sc4ccccc4c23)cc1 ; c1ccc(-c2ccc3c(c2)sc2ccc(N(c4cccc5sc6ccccc6c45)c4ccc(-c5ccccc5)c5sc6ccccc6c45)cc23)cc1]

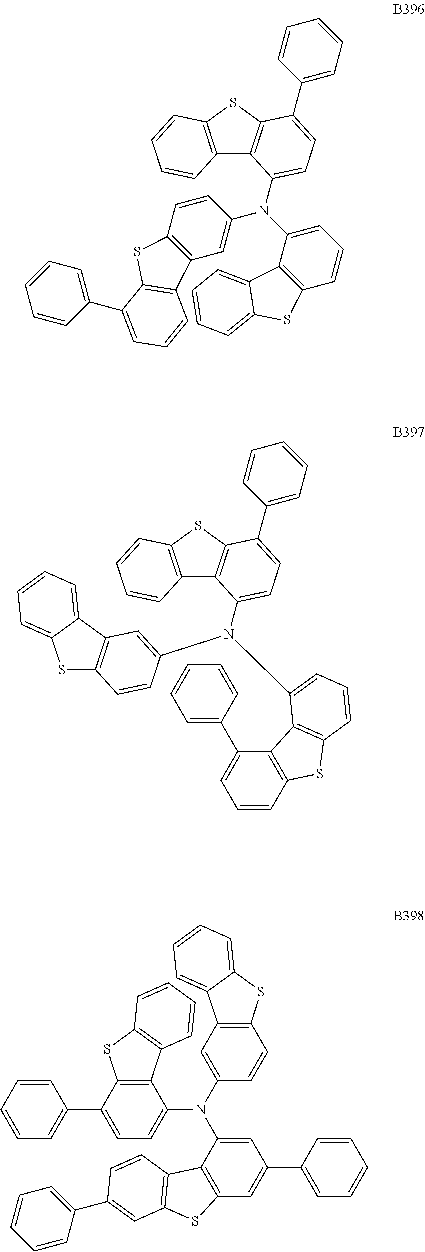 [SMILES: c1ccc(-c2cccc3c2sc2ccc(N(c4cccc5sc6ccccc6c45)c4ccc(-c5ccccc5)c5sc6ccccc6c45)cc23)cc1 ; c1ccc(-c2ccc(N(c3ccc4sc5ccccc5c4c3)c3cccc4sc5cccc(-c6ccccc6)c5c34)c3c2sc2ccccc23)cc1 ; c1ccc(-c2ccc3c(c2)sc2cc(-c4ccccc4)cc(N(c4ccc5sc6ccccc6c5c4)c4ccc(-c5ccccc5)c5sc6ccccc6c45)c23)cc1]

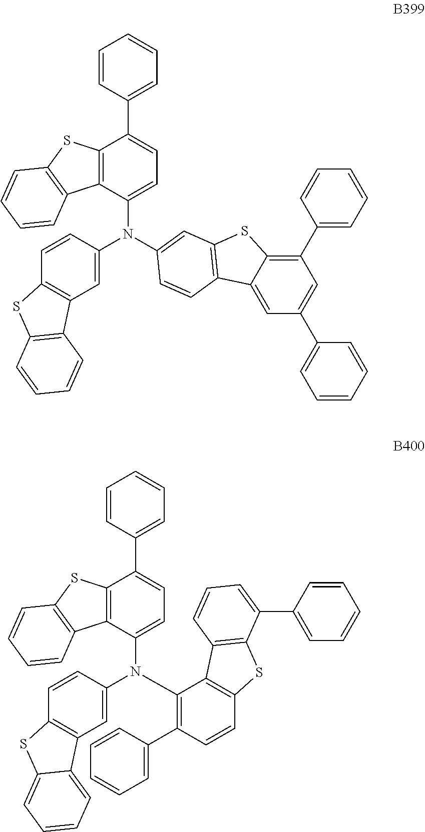 [SMILES: c1ccc(-c2cc(-c3ccccc3)c3sc4cc(N(c5ccc6sc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3sc4ccccc4c3c2)c2ccc(-c3ccccc3)c3sc4ccccc4c23)cc1]

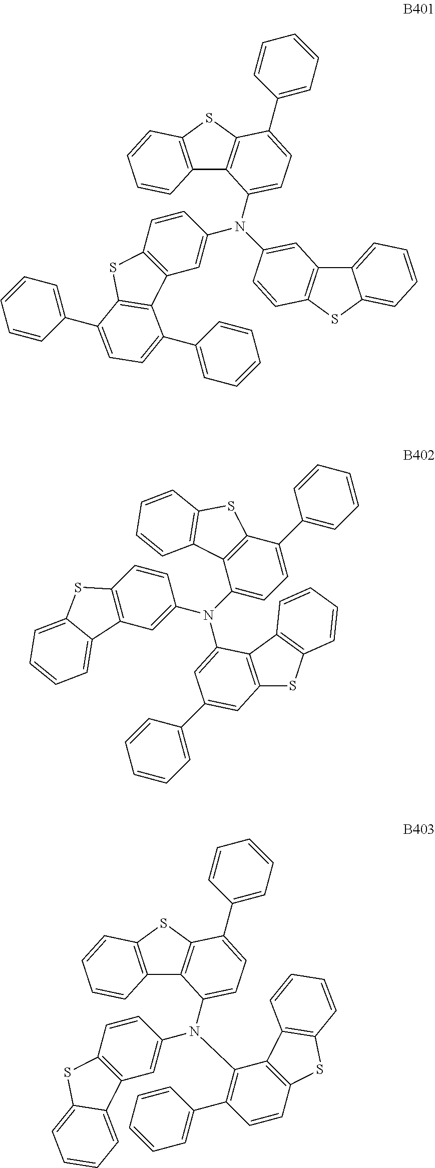 [SMILES: c1ccc(-c2ccc(-c3ccccc3)c3c2sc2ccc(N(c4ccc5sc6ccccc6c5c4)c4ccc(-c5ccccc5)c5sc6ccccc6c45)cc23)cc1 ; c1ccc(-c2cc(N(c3ccc4sc5ccccc5c4c3)c3ccc(-c4ccccc4)c4sc5ccccc5c34)c3c(c2)sc2ccccc23)cc1 ; c1ccc(-c2ccc3sc4ccccc4c3c2N(c2ccc3sc4ccccc4c3c2)c2ccc(-c3ccccc3)c3sc4ccccc4c23)cc1]

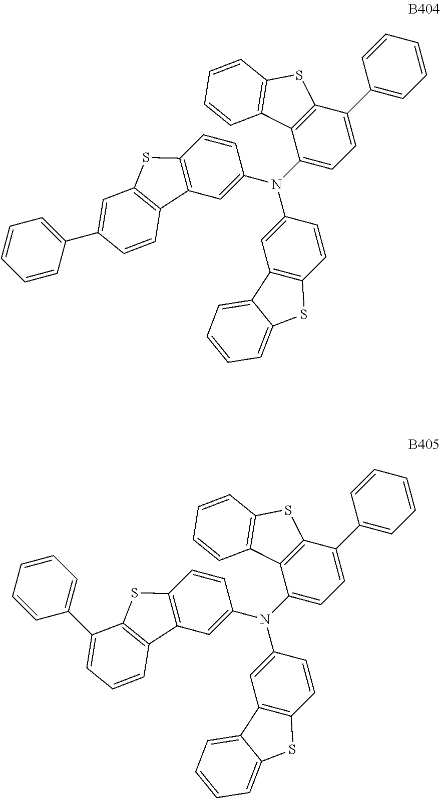 [SMILES: c1ccc(-c2ccc3c(c2)sc2ccc(N(c4ccc5sc6ccccc6c5c4)c4ccc(-c5ccccc5)c5sc6ccccc6c45)cc23)cc1 ; c1ccc(-c2cccc3c2sc2ccc(N(c4ccc5sc6ccccc6c5c4)c4ccc(-c5ccccc5)c5sc6ccccc6c45)cc23)cc1]

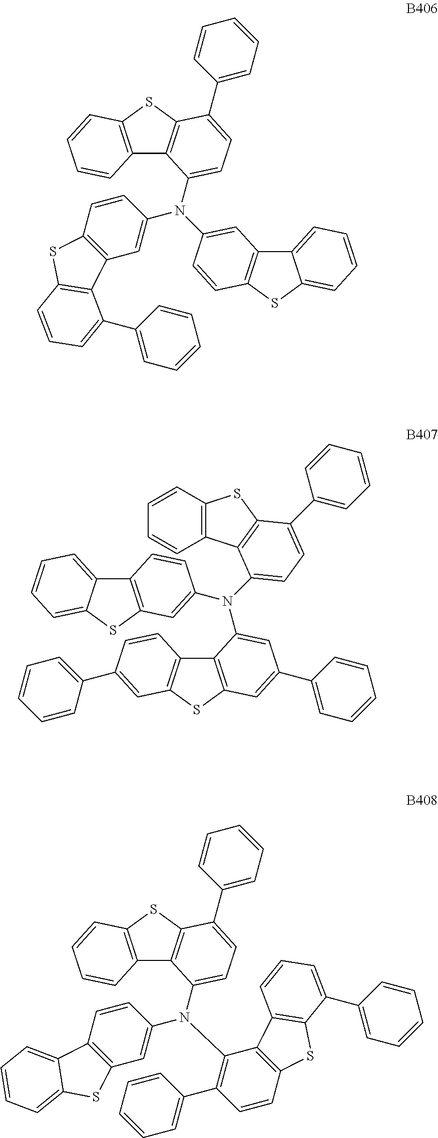 [SMILES: c1ccc(-c2ccc(N(c3ccc4sc5ccccc5c4c3)c3ccc4sc5cccc(-c6ccccc6)c5c4c3)c3c2sc2ccccc23)cc1 ; c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3sc4ccccc4c23)cc1]

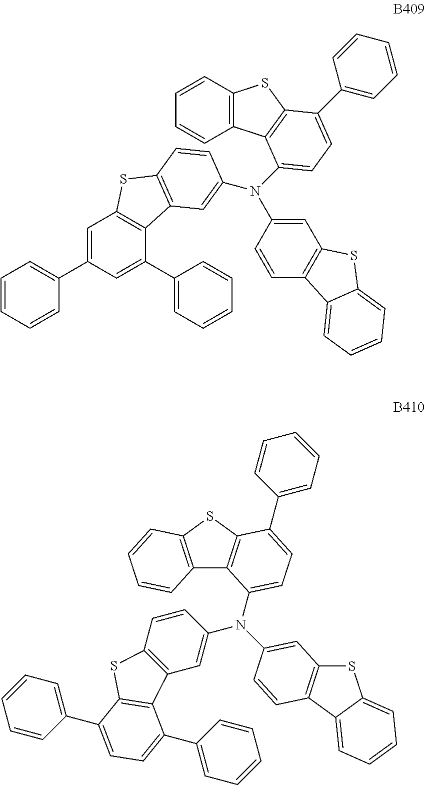 [SMILES: c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1 ; c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1]

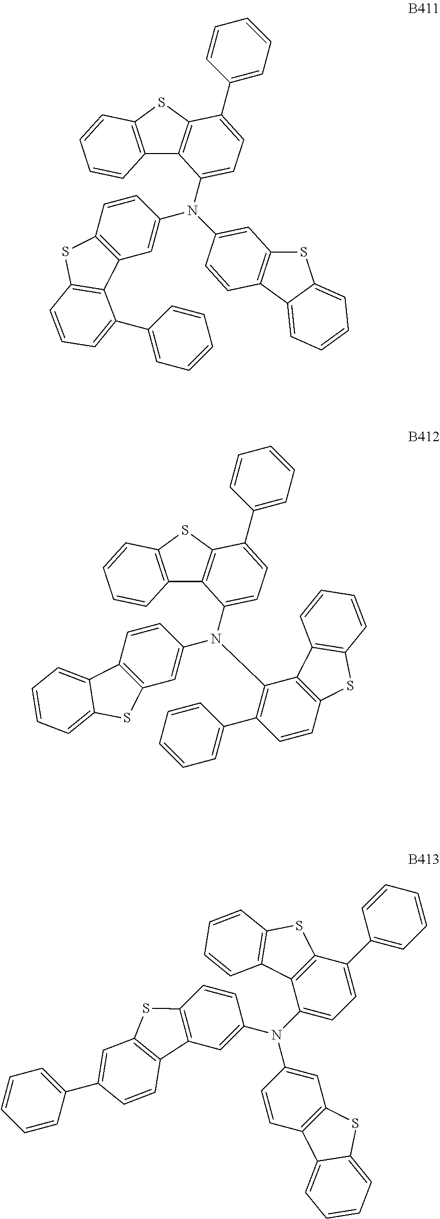 [SMILES: c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1 ; c1ccc(-c2ccc3c(c2)sc2ccc(N(c4ccc5c(c4)sc4ccccc45)c4ccc(-c5ccccc5)c5sc6ccccc6c45)cc23)cc1]

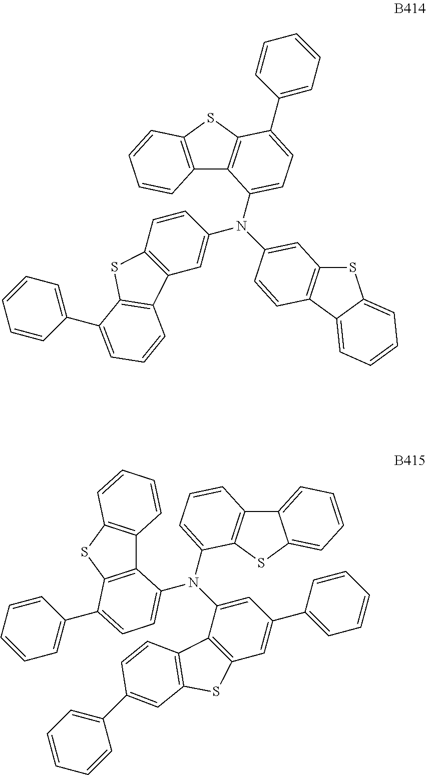 [SMILES: c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4ccc(-c5ccccc5)c5sc6ccccc6c45)c23)cc1]

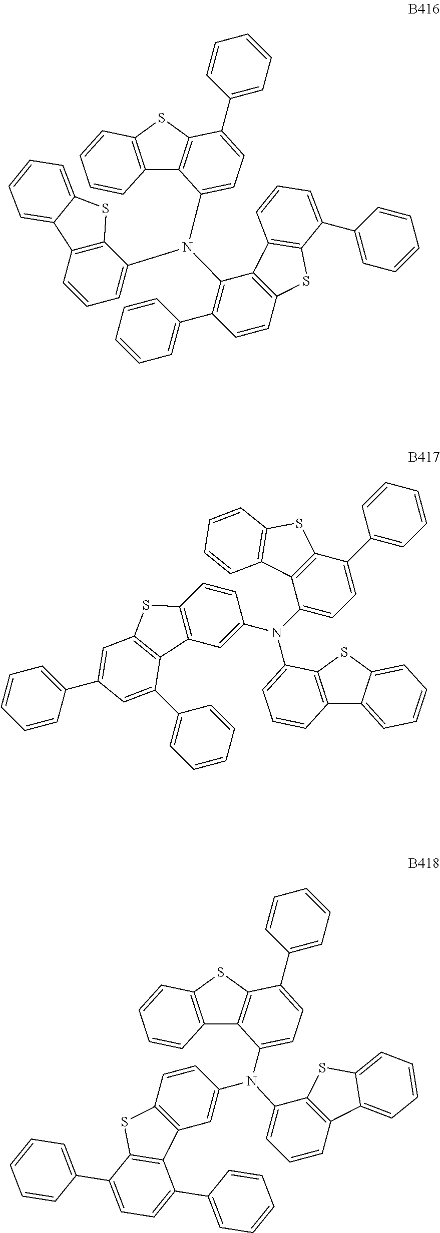 [SMILES: c1ccc(-c2ccc3sc4c(-c5ccccc5)cccc4c3c2N(c2cccc3c2sc2ccccc23)c2ccc(-c3ccccc3)c3sc4ccccc4c23)cc1 ; c1ccc(-c2cc(-c3ccccc3)c3c(c2)sc2ccc(N(c4cccc5c4sc4ccccc45)c4ccc(-c5ccccc5)c5sc6ccccc6c45)cc23)cc1 ; c1ccc(-c2ccc(-c3ccccc3)c3c2sc2ccc(N(c4cccc5c4sc4ccccc45)c4ccc(-c5ccccc5)c5sc6ccccc6c45)cc23)cc1]

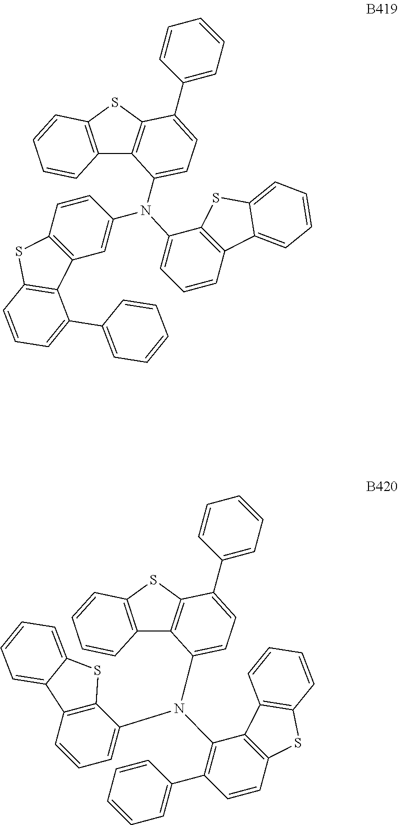 [SMILES: c1ccc(-c2ccc(N(c3ccc4sc5cccc(-c6ccccc6)c5c4c3)c3cccc4c3sc3ccccc34)c3c2sc2ccccc23)cc1 ; c1ccc(-c2ccc3sc4ccccc4c3c2N(c2cccc3c2sc2ccccc23)c2ccc(-c3ccccc3)c3sc4ccccc4c23)cc1]

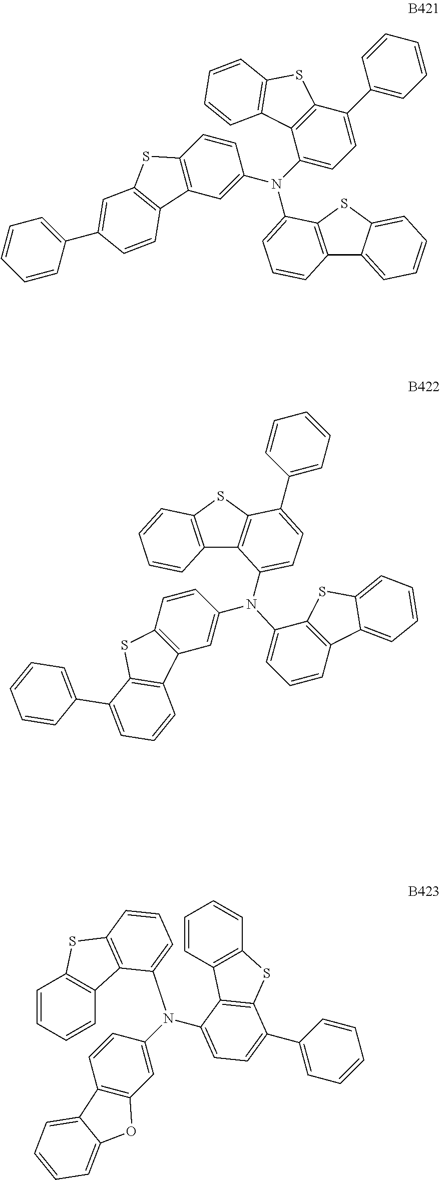 [SMILES: c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2cccc3c2sc2ccc(N(c4cccc5c4sc4ccccc45)c4ccc(-c5ccccc5)c5sc6ccccc6c45)cc23)cc1 ; c1ccc(-c2ccc(N(c3ccc4c(c3)oc3ccccc34)c3cccc4sc5ccccc5c34)c3c2sc2ccccc23)cc1]

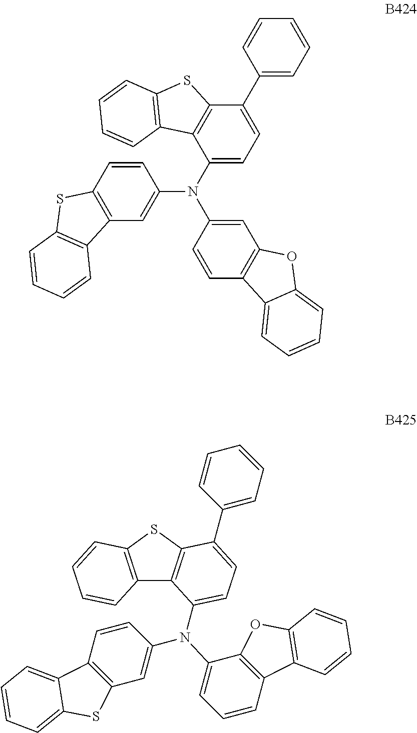 [SMILES: c1ccc(-c2ccc(N(c3ccc4c(c3)oc3ccccc34)c3ccc4sc5ccccc5c4c3)c3c2sc2ccccc23)cc1 ; c1ccc(-c2ccc(N(c3ccc4c(c3)sc3ccccc34)c3cccc4c3oc3ccccc34)c3c2sc2ccccc23)cc1]

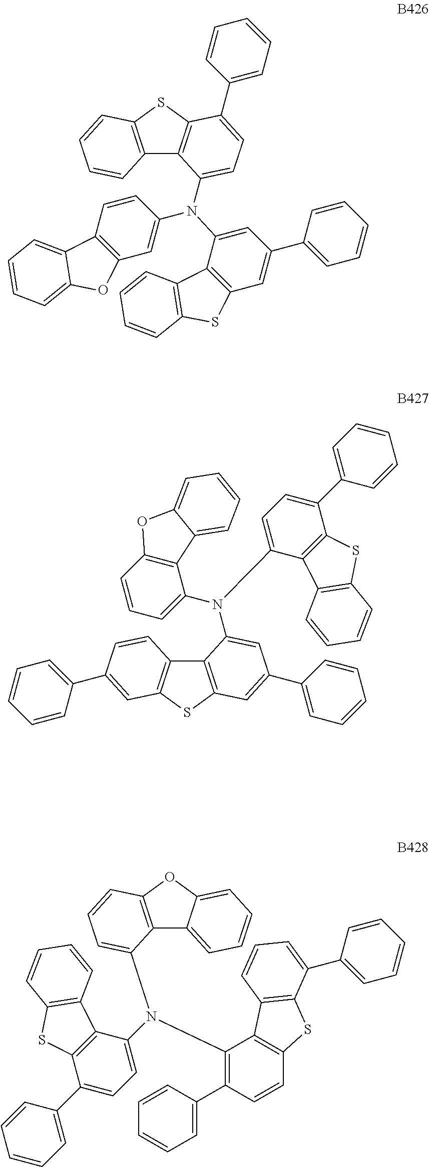 [SMILES: c1ccc(-c2cc(N(c3ccc4c(c3)oc3ccccc34)c3ccc(-c4ccccc4)c4sc5ccccc5c34)c3c(c2)sc2ccccc23)cc1 ; c1ccc(-c2ccc3c(c2)sc2cc(-c4ccccc4)cc(N(c4cccc5oc6ccccc6c45)c4ccc(-c5ccccc5)c5sc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3sc4ccccc4c23)cc1]

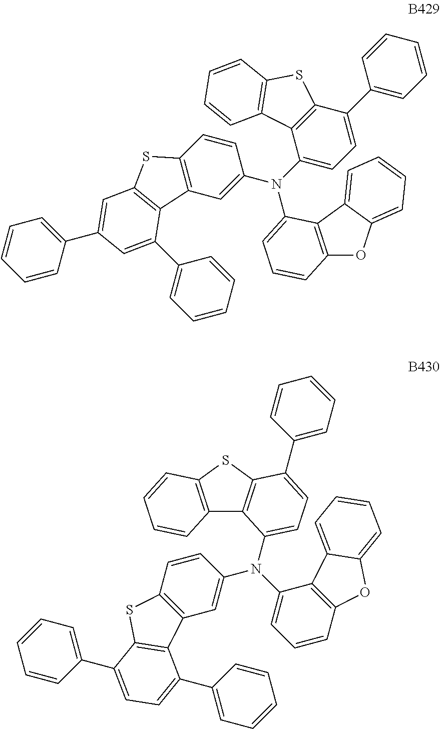 [SMILES: c1ccc(-c2cc(-c3ccccc3)c3c(c2)sc2ccc(N(c4cccc5oc6ccccc6c45)c4ccc(-c5ccccc5)c5sc6ccccc6c45)cc23)cc1 ; c1ccc(-c2ccc(-c3ccccc3)c3c2sc2ccc(N(c4cccc5oc6ccccc6c45)c4ccc(-c5ccccc5)c5sc6ccccc6c45)cc23)cc1]

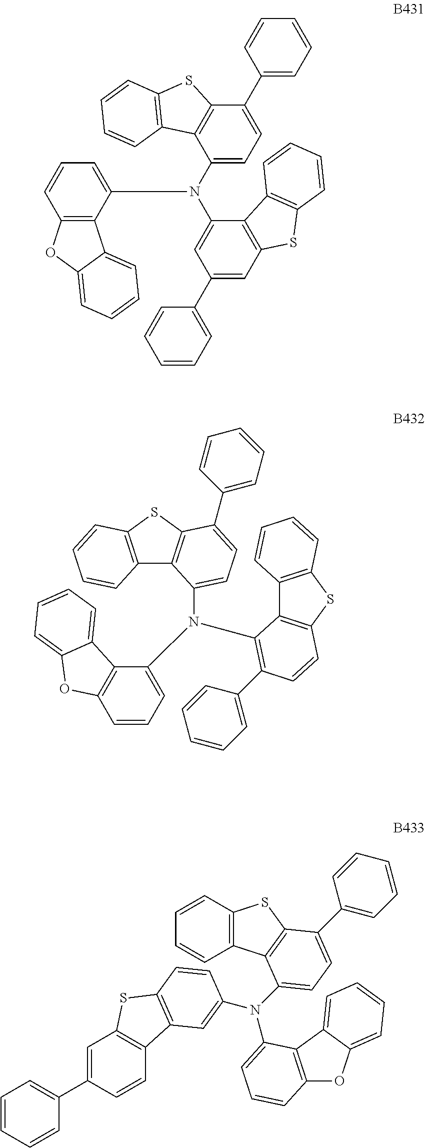 [SMILES: c1ccc(-c2cc(N(c3cccc4oc5ccccc5c34)c3ccc(-c4ccccc4)c4sc5ccccc5c34)c3c(c2)sc2ccccc23)cc1 ; c1ccc(-c2ccc3sc4ccccc4c3c2N(c2cccc3oc4ccccc4c23)c2ccc(-c3ccccc3)c3sc4ccccc4c23)cc1 ; c1ccc(-c2ccc3c(c2)sc2ccc(N(c4cccc5oc6ccccc6c45)c4ccc(-c5ccccc5)c5sc6ccccc6c45)cc23)cc1]

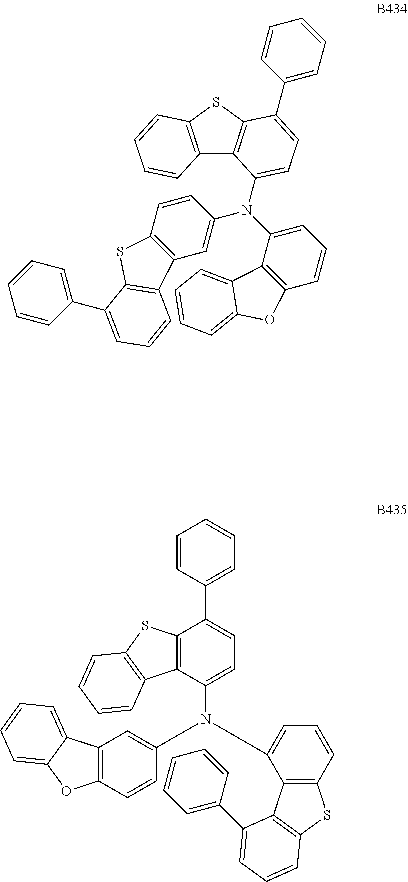 [SMILES: c1ccc(-c2cccc3c2sc2ccc(N(c4cccc5oc6ccccc6c45)c4ccc(-c5ccccc5)c5sc6ccccc6c45)cc23)cc1 ; c1ccc(-c2ccc(N(c3ccc4oc5ccccc5c4c3)c3cccc4sc5cccc(-c6ccccc6)c5c34)c3c2sc2ccccc23)cc1]

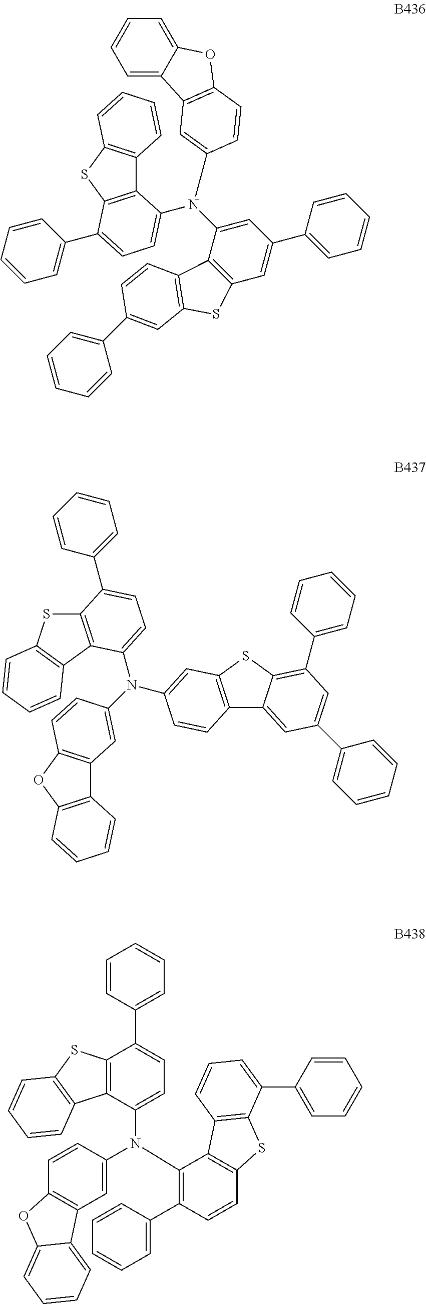 [SMILES: c1ccc(-c2ccc3c(c2)sc2cc(-c4ccccc4)cc(N(c4ccc5oc6ccccc6c5c4)c4ccc(-c5ccccc5)c5sc6ccccc6c45)c23)cc1 ; c1ccc(-c2cc(-c3ccccc3)c3sc4cc(N(c5ccc6oc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3oc4ccccc4c3c2)c2ccc(-c3ccccc3)c3sc4ccccc4c23)cc1]

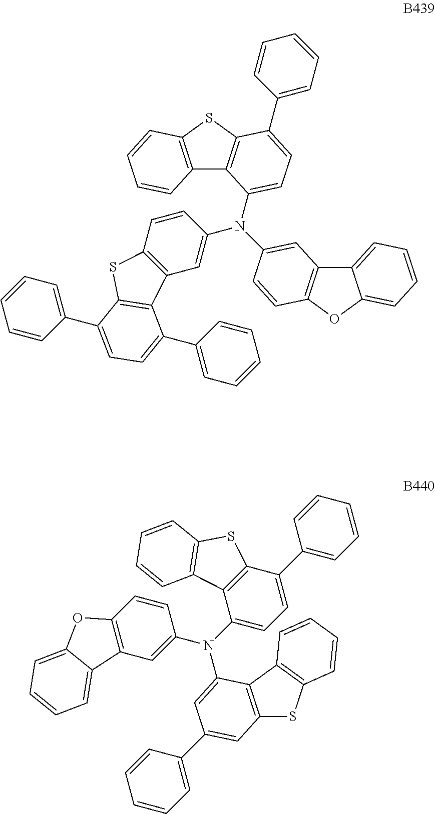 [SMILES: c1ccc(-c2ccc(-c3ccccc3)c3c2sc2ccc(N(c4ccc5oc6ccccc6c5c4)c4ccc(-c5ccccc5)c5sc6ccccc6c45)cc23)cc1 ; c1ccc(-c2cc(N(c3ccc4oc5ccccc5c4c3)c3ccc(-c4ccccc4)c4sc5ccccc5c34)c3c(c2)sc2ccccc23)cc1]

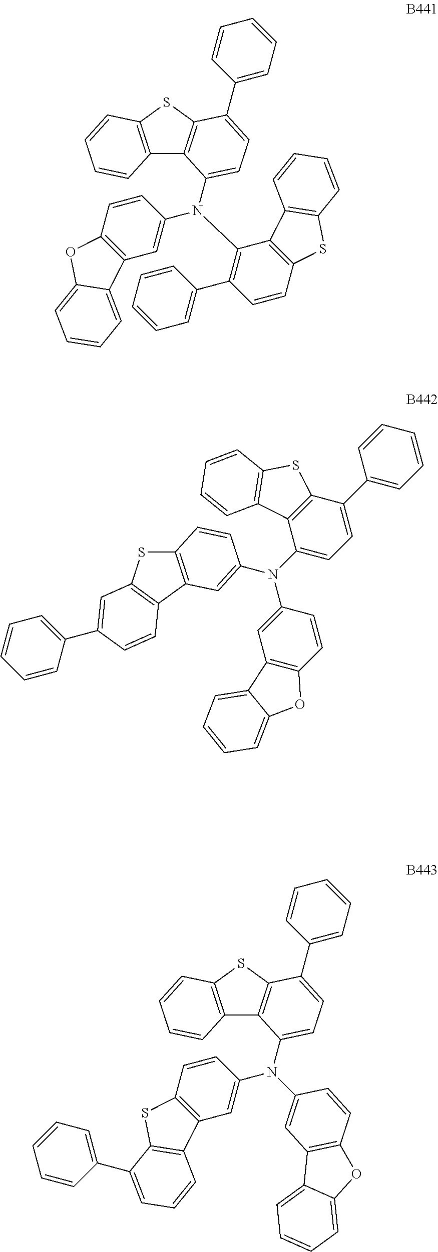 [SMILES: c1ccc(-c2ccc3sc4ccccc4c3c2N(c2ccc3oc4ccccc4c3c2)c2ccc(-c3ccccc3)c3sc4ccccc4c23)cc1 ; c1ccc(-c2ccc3c(c2)sc2ccc(N(c4ccc5oc6ccccc6c5c4)c4ccc(-c5ccccc5)c5sc6ccccc6c45)cc23)cc1 ; c1ccc(-c2cccc3c2sc2ccc(N(c4ccc5oc6ccccc6c5c4)c4ccc(-c5ccccc5)c5sc6ccccc6c45)cc23)cc1]

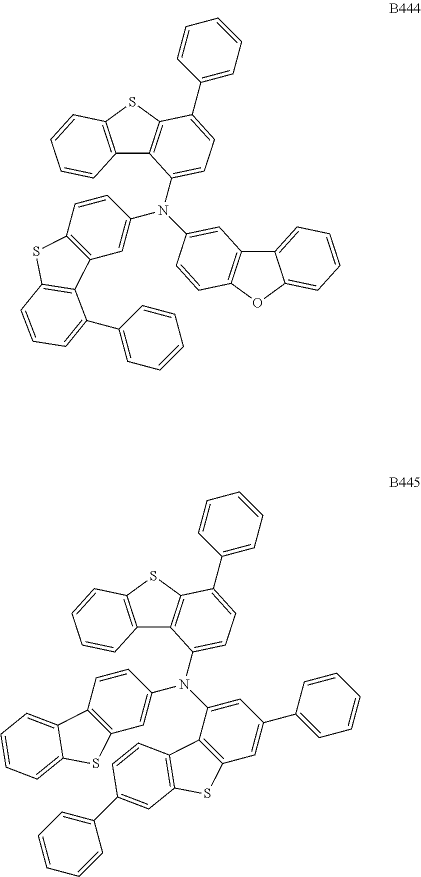 [SMILES: c1ccc(-c2ccc(N(c3ccc4oc5ccccc5c4c3)c3ccc4sc5cccc(-c6ccccc6)c5c4c3)c3c2sc2ccccc23)cc1 ; c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1]

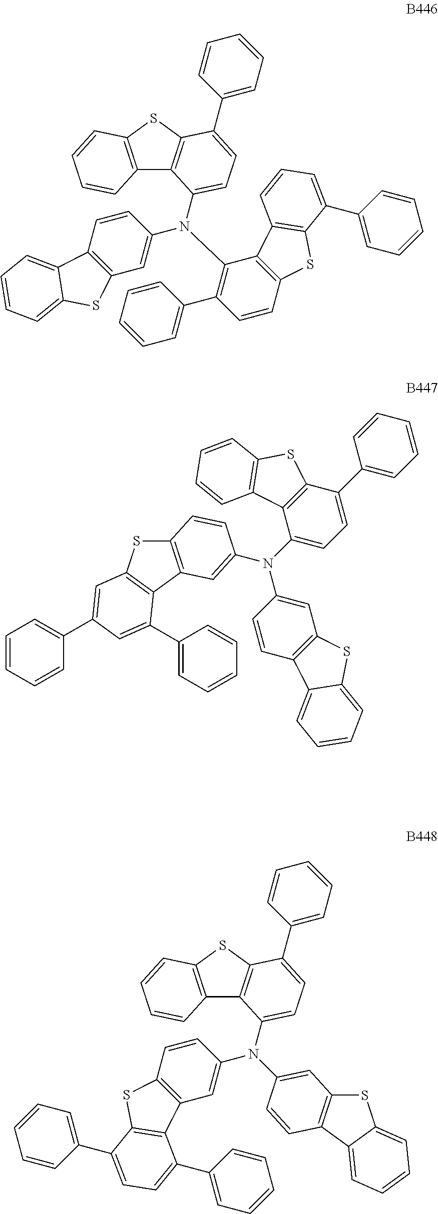 [SMILES: c1ccc(-c2ccc3sc4c(-c5ccccc5)cccc4c3c2N(c2ccc3c(c2)sc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1 ; c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1]

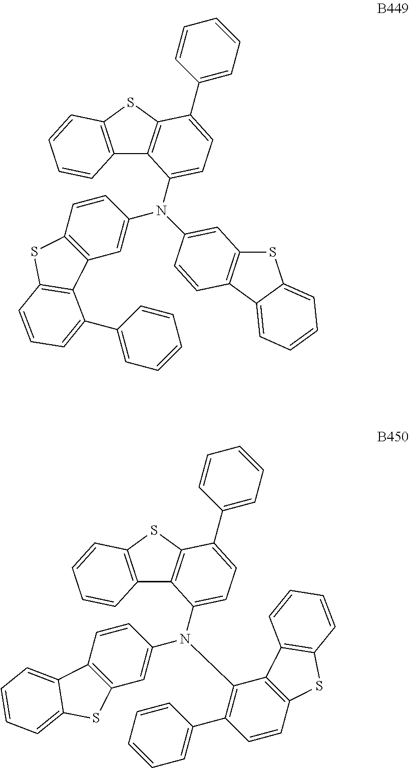 [SMILES: c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1]

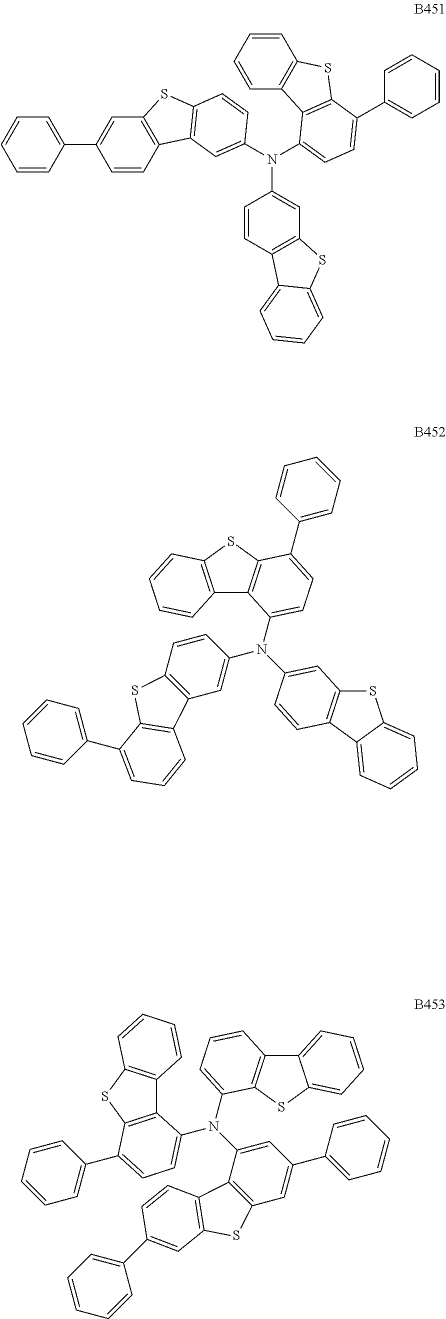 [SMILES: c1ccc(-c2ccc3c(c2)sc2ccc(N(c4ccc5c(c4)sc4ccccc45)c4ccc(-c5ccccc5)c5sc6ccccc6c45)cc23)cc1 ; c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4ccc(-c5ccccc5)c5sc6ccccc6c45)c23)cc1]

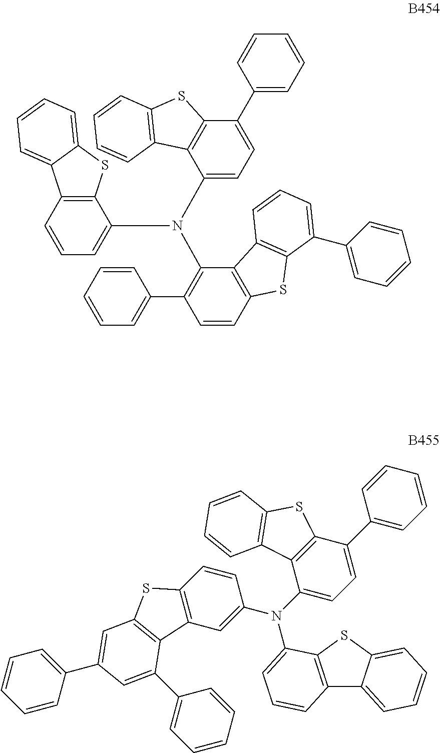 [SMILES: c1ccc(-c2ccc3sc4c(-c5ccccc5)cccc4c3c2N(c2cccc3c2sc2ccccc23)c2ccc(-c3ccccc3)c3sc4ccccc4c23)cc1 ; c1ccc(-c2cc(-c3ccccc3)c3c(c2)sc2ccc(N(c4cccc5c4sc4ccccc45)c4ccc(-c5ccccc5)c5sc6ccccc6c45)cc23)cc1]

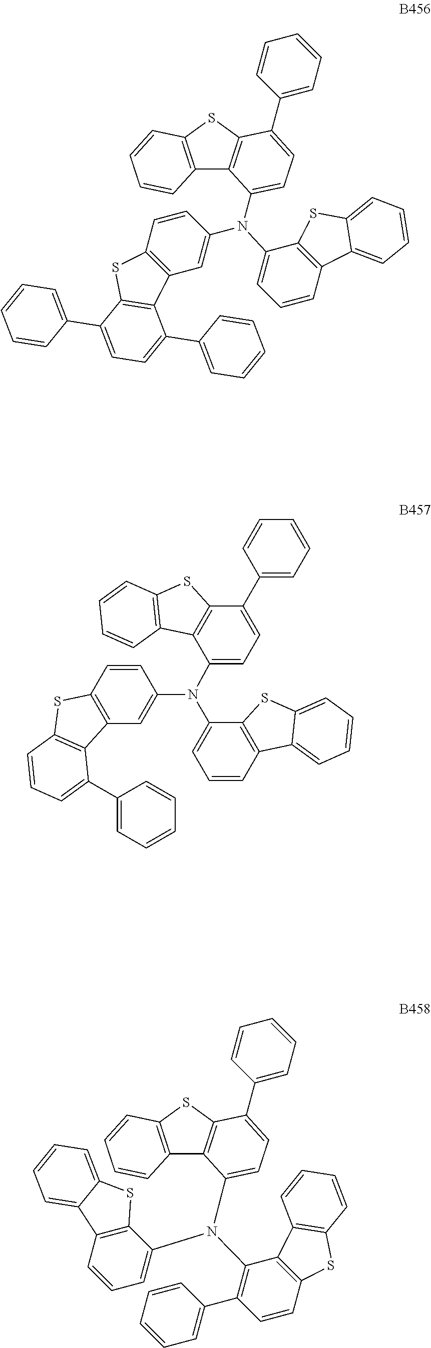 [SMILES: c1ccc(-c2ccc(-c3ccccc3)c3c2sc2ccc(N(c4cccc5c4sc4ccccc45)c4ccc(-c5ccccc5)c5sc6ccccc6c45)cc23)cc1 ; c1ccc(-c2ccc(N(c3ccc4sc5cccc(-c6ccccc6)c5c4c3)c3cccc4c3sc3ccccc34)c3c2sc2ccccc23)cc1 ; c1ccc(-c2ccc3sc4ccccc4c3c2N(c2cccc3c2sc2ccccc23)c2ccc(-c3ccccc3)c3sc4ccccc4c23)cc1]

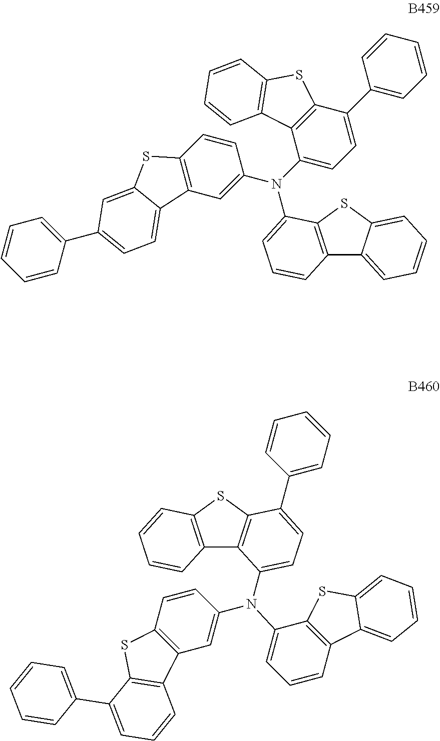 [SMILES: c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2cccc3c2sc2ccc(N(c4cccc5c4sc4ccccc45)c4ccc(-c5ccccc5)c5sc6ccccc6c45)cc23)cc1]

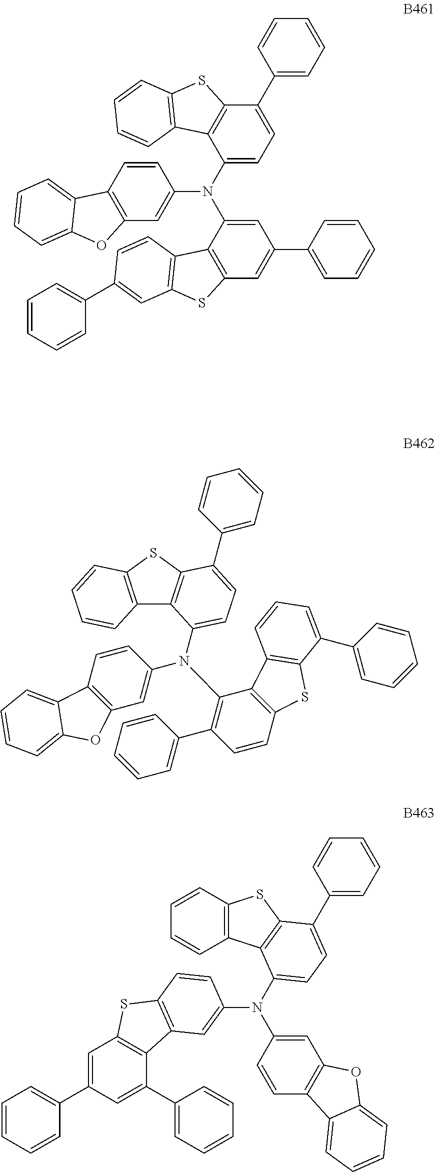 [SMILES: c1ccc(-c2ccc3c(c2)sc2cc(-c4ccccc4)cc(N(c4ccc5c(c4)oc4ccccc45)c4ccc(-c5ccccc5)c5sc6ccccc6c45)c23)cc1 ; c1ccc(-c2ccc3sc4c(-c5ccccc5)cccc4c3c2N(c2ccc3c(c2)oc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)oc4ccccc45)c4ccc(-c5ccccc5)c5sc6ccccc6c45)cc23)cc1]

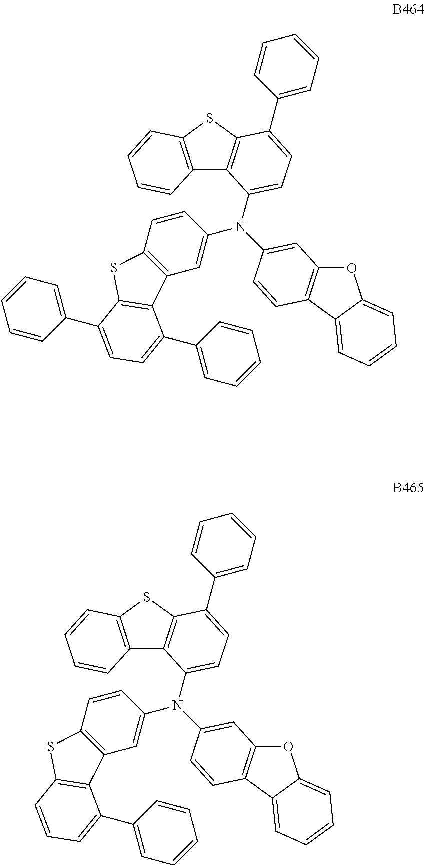 [SMILES: c1ccc(-c2ccc(-c3ccccc3)c3c2sc2ccc(N(c4ccc5c(c4)oc4ccccc45)c4ccc(-c5ccccc5)c5sc6ccccc6c45)cc23)cc1 ; c1ccc(-c2ccc(N(c3ccc4c(c3)oc3ccccc34)c3ccc4sc5cccc(-c6ccccc6)c5c4c3)c3c2sc2ccccc23)cc1]

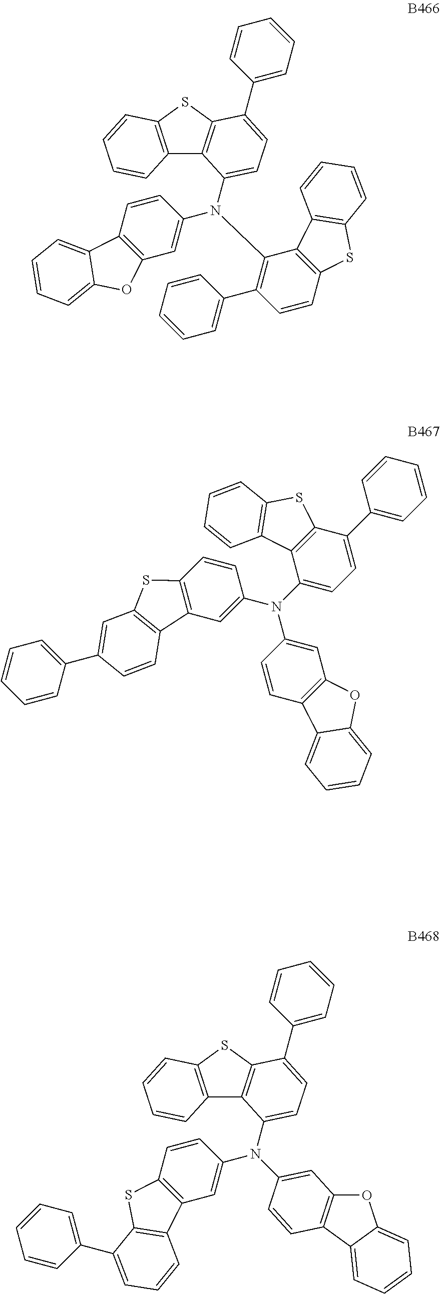 [SMILES: c1ccc(-c2ccc3sc4ccccc4c3c2N(c2ccc3c(c2)oc2ccccc23)c2ccc(-c3ccccc3)c3sc4ccccc4c23)cc1 ; c1ccc(-c2ccc3c(c2)sc2ccc(N(c4ccc5c(c4)oc4ccccc45)c4ccc(-c5ccccc5)c5sc6ccccc6c45)cc23)cc1 ; c1ccc(-c2cccc3c2sc2ccc(N(c4ccc5c(c4)oc4ccccc45)c4ccc(-c5ccccc5)c5sc6ccccc6c45)cc23)cc1]

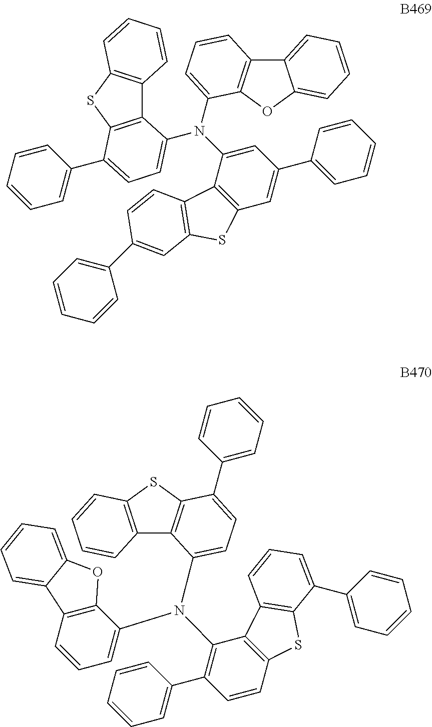 [SMILES: c1ccc(-c2ccc3c(c2)sc2cc(-c4ccccc4)cc(N(c4cccc5c4oc4ccccc45)c4ccc(-c5ccccc5)c5sc6ccccc6c45)c23)cc1 ; c1ccc(-c2ccc3sc4c(-c5ccccc5)cccc4c3c2N(c2cccc3c2oc2ccccc23)c2ccc(-c3ccccc3)c3sc4ccccc4c23)cc1]

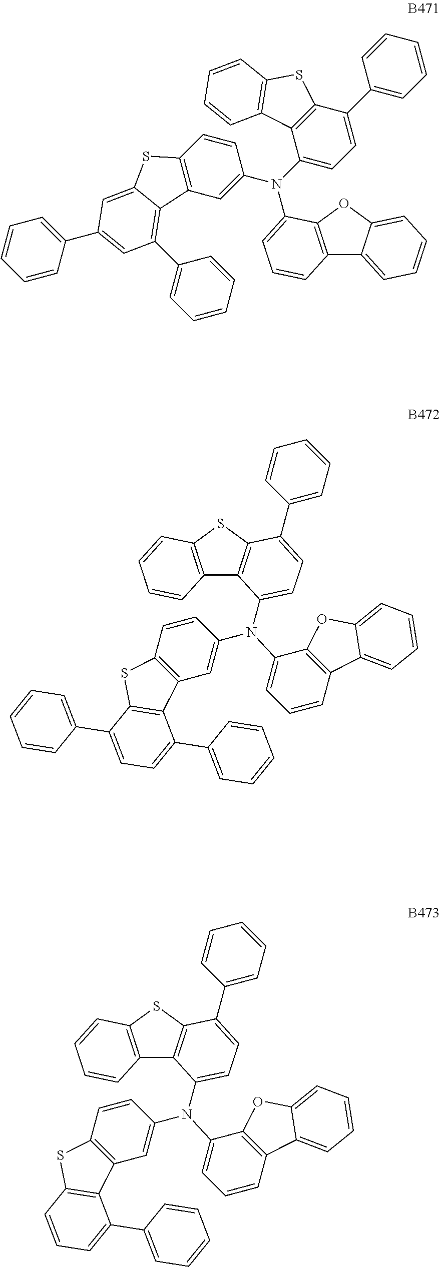 [SMILES: c1ccc(-c2cc(-c3ccccc3)c3c(c2)sc2ccc(N(c4cccc5c4oc4ccccc45)c4ccc(-c5ccccc5)c5sc6ccccc6c45)cc23)cc1 ; c1ccc(-c2ccc(-c3ccccc3)c3c2sc2ccc(N(c4cccc5c4oc4ccccc45)c4ccc(-c5ccccc5)c5sc6ccccc6c45)cc23)cc1 ; c1ccc(-c2ccc(N(c3ccc4sc5cccc(-c6ccccc6)c5c4c3)c3cccc4c3oc3ccccc34)c3c2sc2ccccc23)cc1]

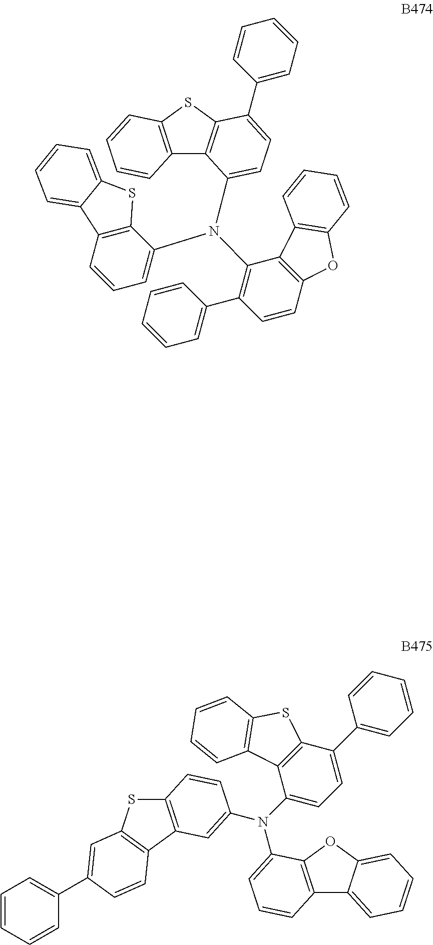 [SMILES: c1ccc(-c2ccc3oc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4oc4ccccc45)c4ccc(-c5ccccc5)c5sc6ccccc6c45)cc23)cc1]

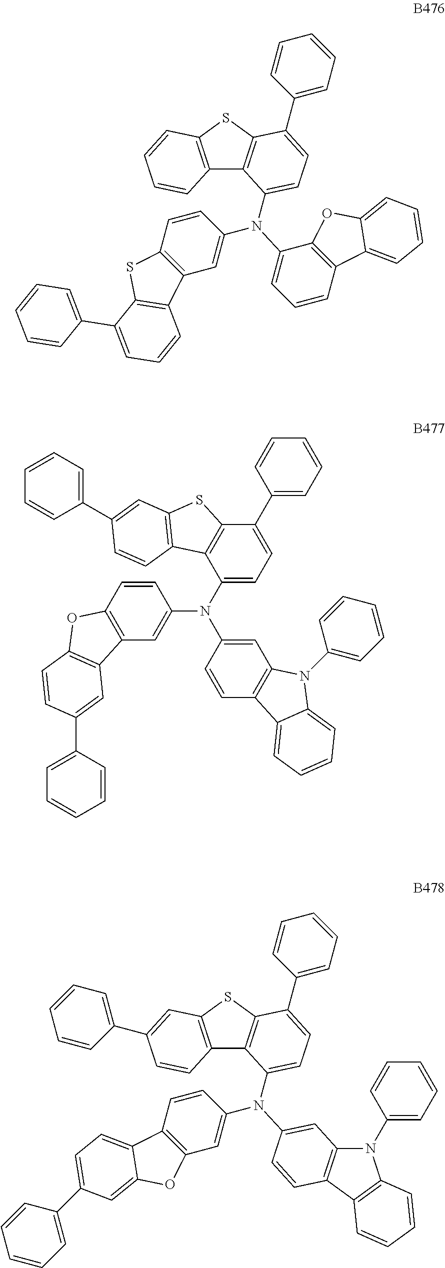 [SMILES: c1ccc(-c2cccc3c2sc2ccc(N(c4cccc5c4oc4ccccc45)c4ccc(-c5ccccc5)c5sc6ccccc6c45)cc23)cc1 ; c1ccc(-c2ccc3c(c2)sc2c(-c4ccccc4)ccc(N(c4ccc5oc6ccc(-c7ccccc7)cc6c5c4)c4ccc5c6ccccc6n(-c6ccccc6)c5c4)c23)cc1 ; c1ccc(-c2ccc3c(c2)oc2cc(N(c4ccc5c6ccccc6n(-c6ccccc6)c5c4)c4ccc(-c5ccccc5)c5sc6cc(-c7ccccc7)ccc6c45)ccc23)cc1]

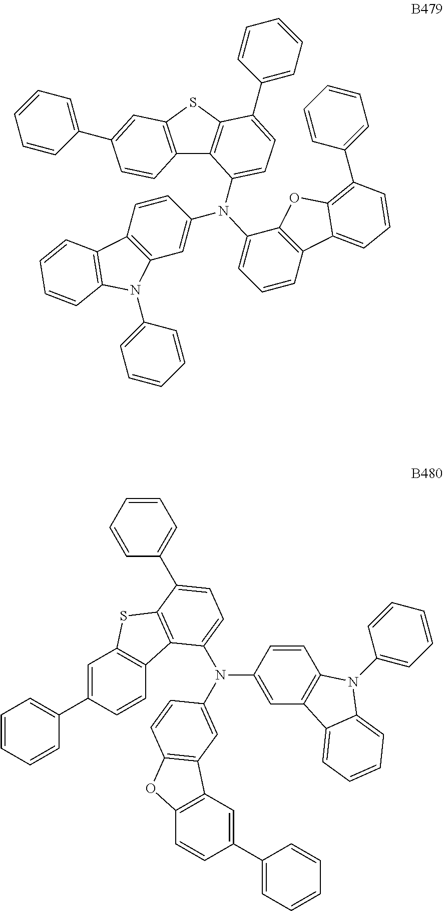 [SMILES: c1ccc(-c2ccc3c(c2)sc2c(-c4ccccc4)ccc(N(c4ccc5c6ccccc6n(-c6ccccc6)c5c4)c4cccc5c4oc4c(-c6ccccc6)cccc45)c23)cc1 ; c1ccc(-c2ccc3c(c2)sc2c(-c4ccccc4)ccc(N(c4ccc5oc6ccc(-c7ccccc7)cc6c5c4)c4ccc5c(c4)c4ccccc4n5-c4ccccc4)c23)cc1]

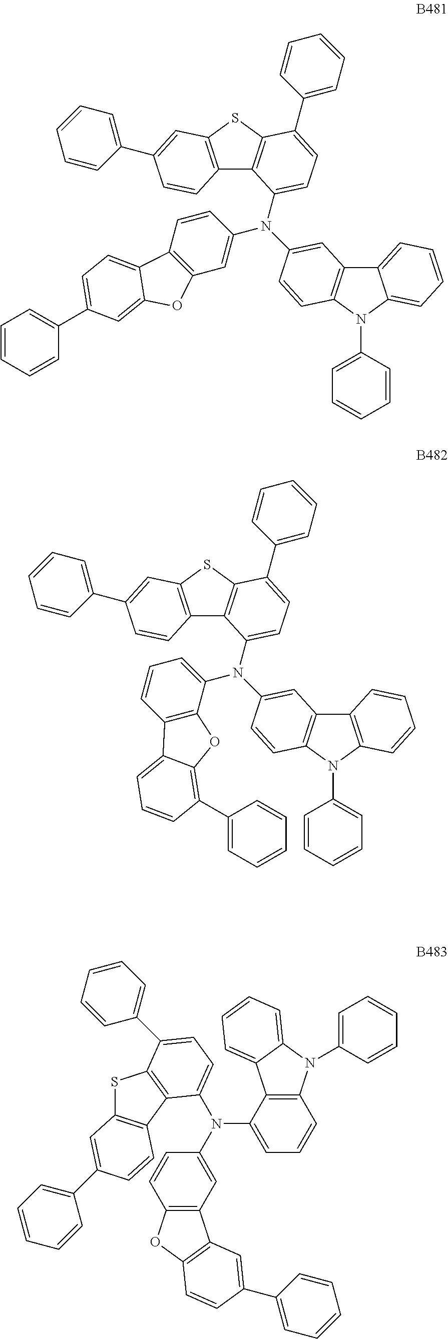 [SMILES: c1ccc(-c2ccc3c(c2)oc2cc(N(c4ccc5c(c4)c4ccccc4n5-c4ccccc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c(c4)c4ccccc4n5-c4ccccc4)c4cccc5c4oc4c(-c6ccccc6)cccc45)c23)cc1 ; c1ccc(-c2ccc3c(c2)sc2c(-c4ccccc4)ccc(N(c4ccc5oc6ccc(-c7ccccc7)cc6c5c4)c4cccc5c4c4ccccc4n5-c4ccccc4)c23)cc1]

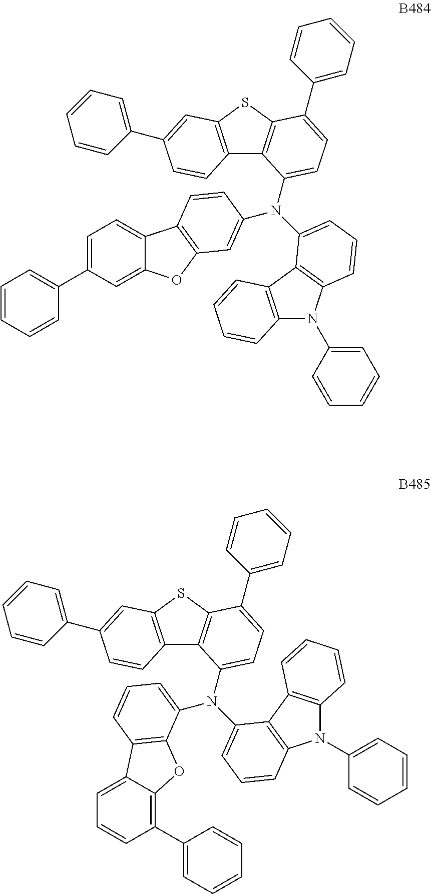 [SMILES: c1ccc(-c2ccc3c(c2)oc2cc(N(c4ccc(-c5ccccc5)c5sc6cc(-c7ccccc7)ccc6c45)c4cccc5c4c4ccccc4n5-c4ccccc4)ccc23)cc1 ; c1ccc(-c2ccc3c(c2)sc2c(-c4ccccc4)ccc(N(c4cccc5c4oc4c(-c6ccccc6)cccc45)c4cccc5c4c4ccccc4n5-c4ccccc4)c23)cc1]

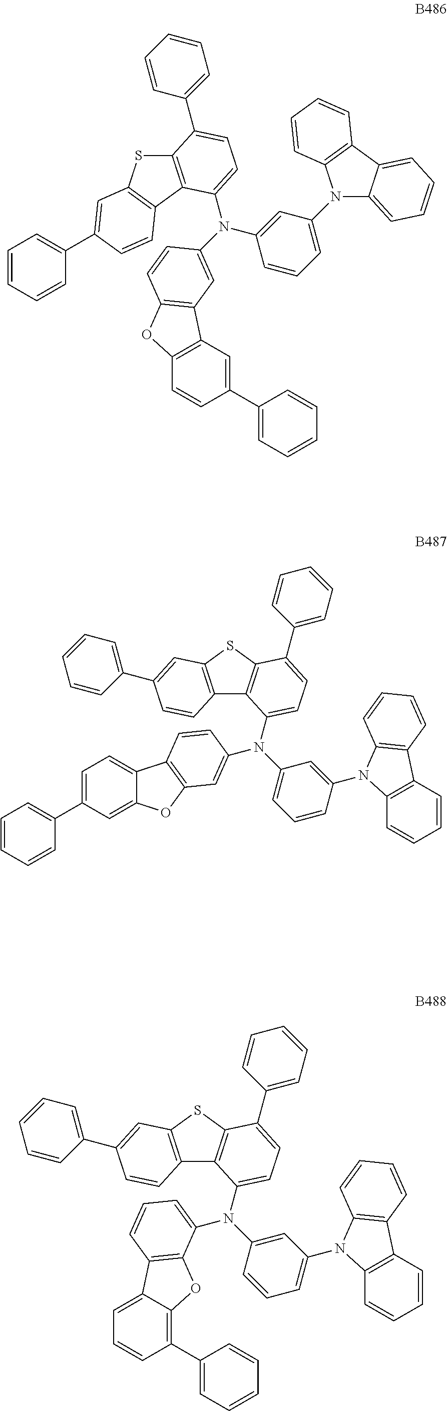 [SMILES: c1ccc(-c2ccc3c(c2)sc2c(-c4ccccc4)ccc(N(c4cccc(-n5c6ccccc6c6ccccc65)c4)c4ccc5oc6ccc(-c7ccccc7)cc6c5c4)c23)cc1 ; c1ccc(-c2ccc3c(c2)oc2cc(N(c4cccc(-n5c6ccccc6c6ccccc65)c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4cccc(-n5c6ccccc6c6ccccc65)c4)c4cccc5c4oc4c(-c6ccccc6)cccc45)c23)cc1]

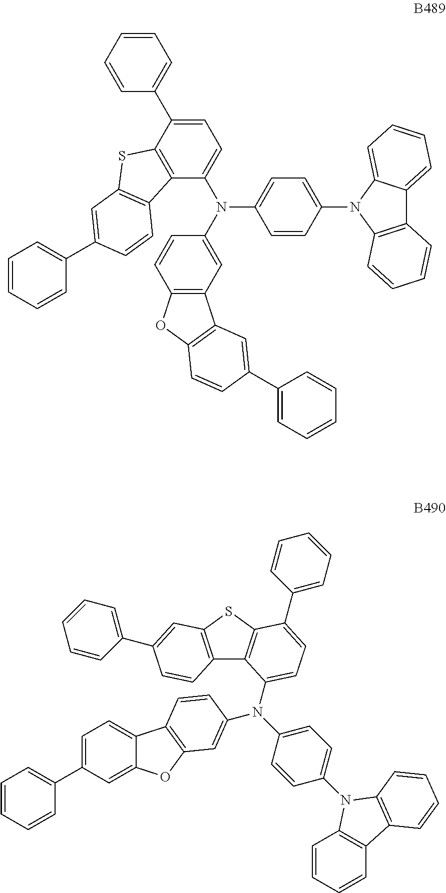 [SMILES: c1ccc(-c2ccc3c(c2)sc2c(-c4ccccc4)ccc(N(c4ccc(-n5c6ccccc6c6ccccc65)cc4)c4ccc5oc6ccc(-c7ccccc7)cc6c5c4)c23)cc1 ; c1ccc(-c2ccc3c(c2)oc2cc(N(c4ccc(-n5c6ccccc6c6ccccc65)cc4)c4ccc(-c5ccccc5)c5sc6cc(-c7ccccc7)ccc6c45)ccc23)cc1]

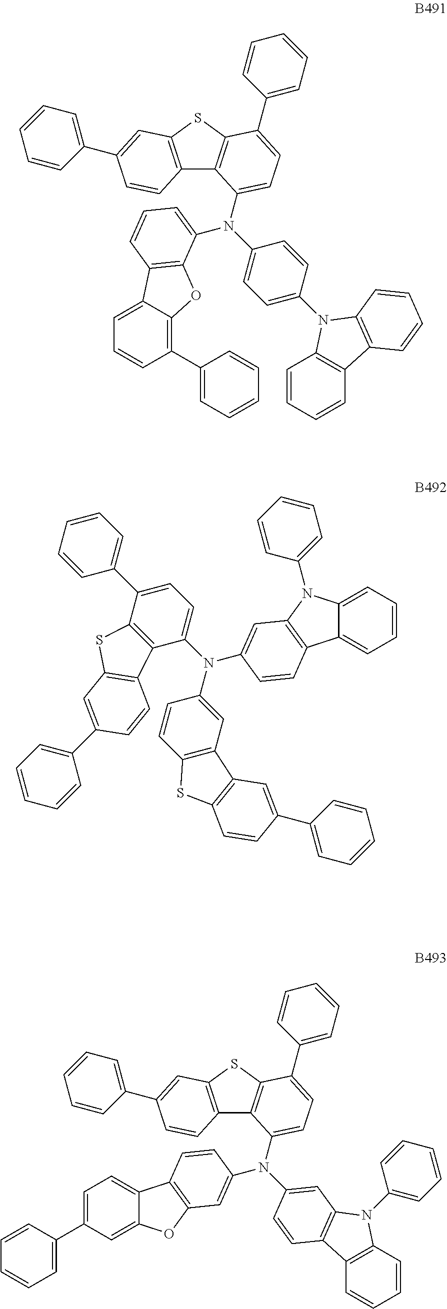 [SMILES: c1ccc(-c2ccc3c(c2)sc2c(-c4ccccc4)ccc(N(c4ccc(-n5c6ccccc6c6ccccc65)cc4)c4cccc5c4oc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4ccc5c6ccccc6n(-c6ccccc6)c5c4)c23)cc1 ; c1ccc(-c2ccc3c(c2)oc2cc(N(c4ccc5c6ccccc6n(-c6ccccc6)c5c4)c4ccc(-c5ccccc5)c5sc6cc(-c7ccccc7)ccc6c45)ccc23)cc1]

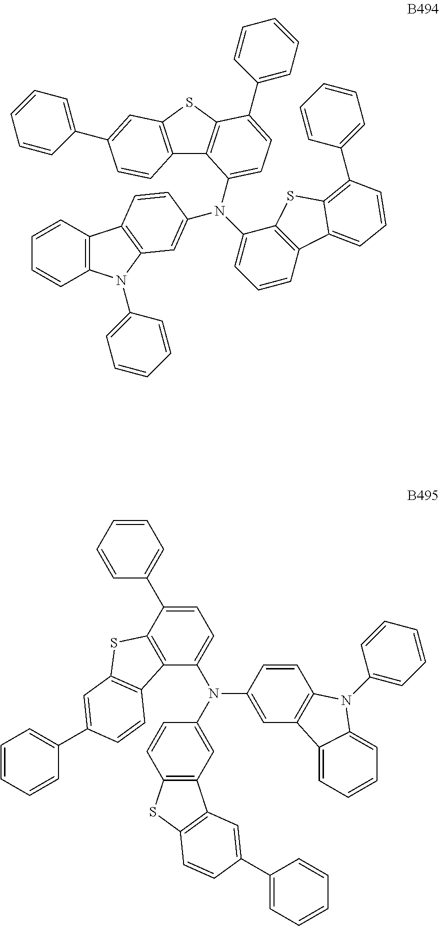 [SMILES: c1ccc(-c2ccc3c(c2)sc2c(-c4ccccc4)ccc(N(c4ccc5c6ccccc6n(-c6ccccc6)c5c4)c4cccc5c4sc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4ccc5c(c4)c4ccccc4n5-c4ccccc4)c23)cc1]

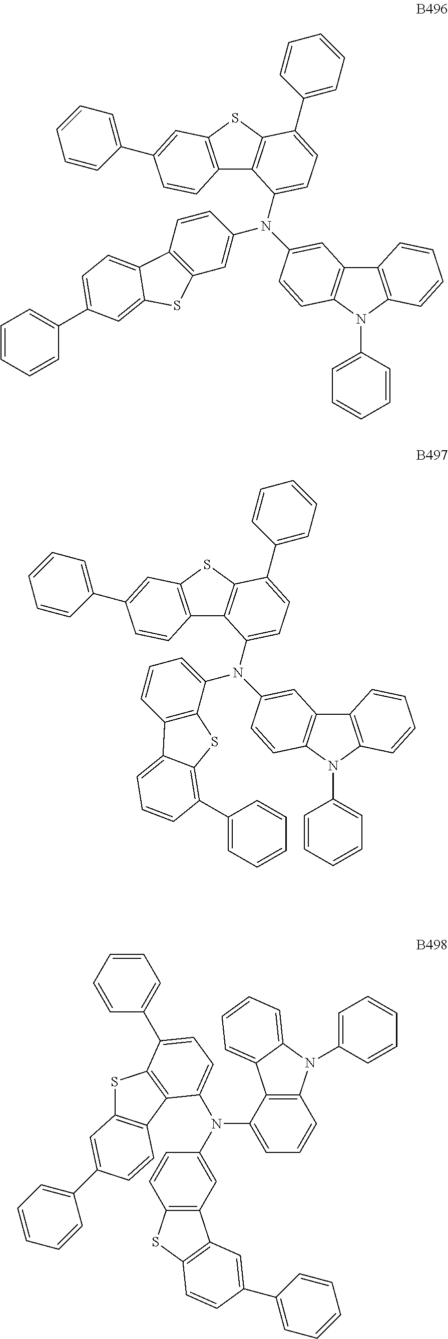 [SMILES: c1ccc(-c2ccc3c(c2)sc2cc(N(c4ccc5c(c4)c4ccccc4n5-c4ccccc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c(c4)c4ccccc4n5-c4ccccc4)c4cccc5c4sc4c(-c6ccccc6)cccc45)c23)cc1 ; c1ccc(-c2ccc3c(c2)sc2c(-c4ccccc4)ccc(N(c4ccc5sc6ccc(-c7ccccc7)cc6c5c4)c4cccc5c4c4ccccc4n5-c4ccccc4)c23)cc1]

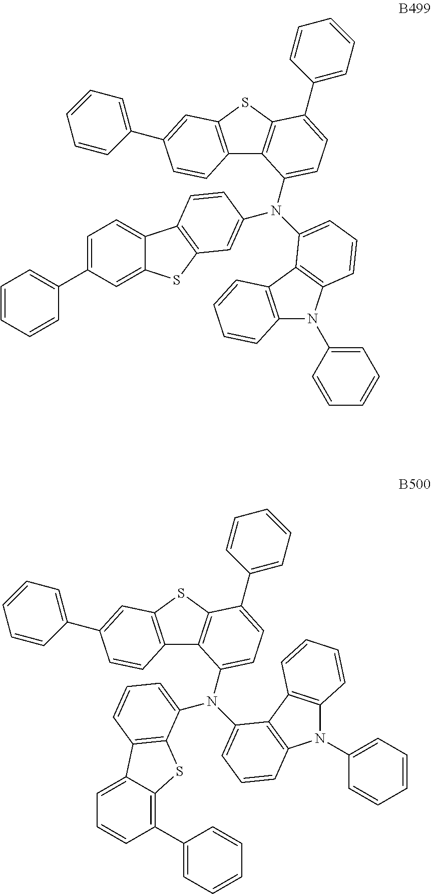 [SMILES: c1ccc(-c2ccc3c(c2)sc2cc(N(c4ccc(-c5ccccc5)c5sc6cc(-c7ccccc7)ccc6c45)c4cccc5c4c4ccccc4n5-c4ccccc4)ccc23)cc1 ; c1ccc(-c2ccc3c(c2)sc2c(-c4ccccc4)ccc(N(c4cccc5c4sc4c(-c6ccccc6)cccc45)c4cccc5c4c4ccccc4n5-c4ccccc4)c23)cc1]

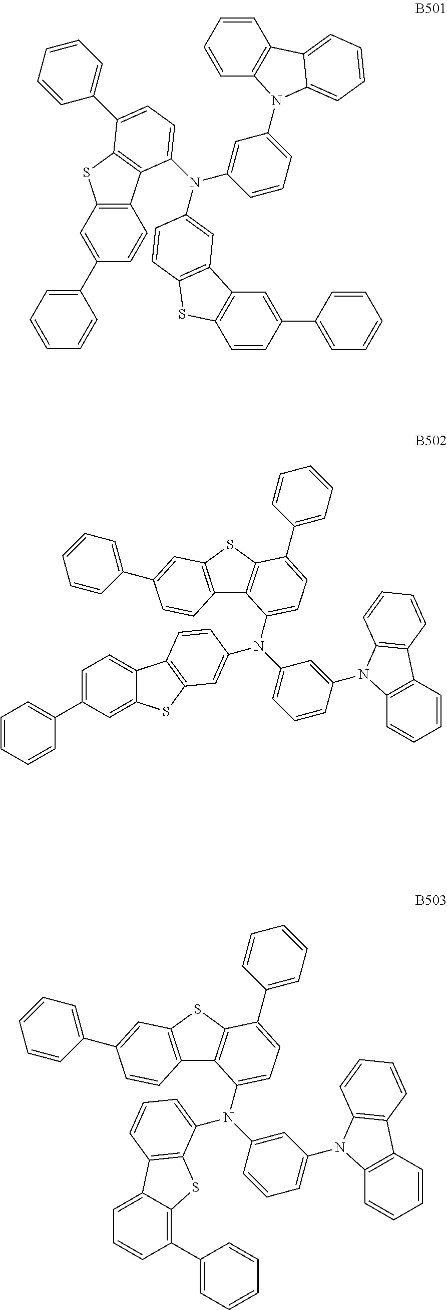 [SMILES: c1ccc(-c2ccc3c(c2)sc2c(-c4ccccc4)ccc(N(c4cccc(-n5c6ccccc6c6ccccc65)c4)c4ccc5sc6ccc(-c7ccccc7)cc6c5c4)c23)cc1 ; c1ccc(-c2ccc3c(c2)sc2cc(N(c4cccc(-n5c6ccccc6c6ccccc65)c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4cccc(-n5c6ccccc6c6ccccc65)c4)c4cccc5c4sc4c(-c6ccccc6)cccc45)c23)cc1]

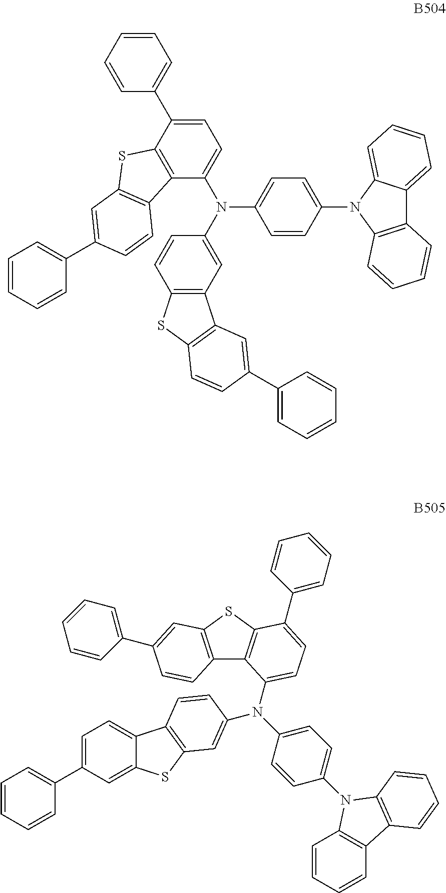 [SMILES: c1ccc(-c2ccc3c(c2)sc2c(-c4ccccc4)ccc(N(c4ccc(-n5c6ccccc6c6ccccc65)cc4)c4ccc5sc6ccc(-c7ccccc7)cc6c5c4)c23)cc1 ; c1ccc(-c2ccc3c(c2)sc2cc(N(c4ccc(-n5c6ccccc6c6ccccc65)cc4)c4ccc(-c5ccccc5)c5sc6cc(-c7ccccc7)ccc6c45)ccc23)cc1]

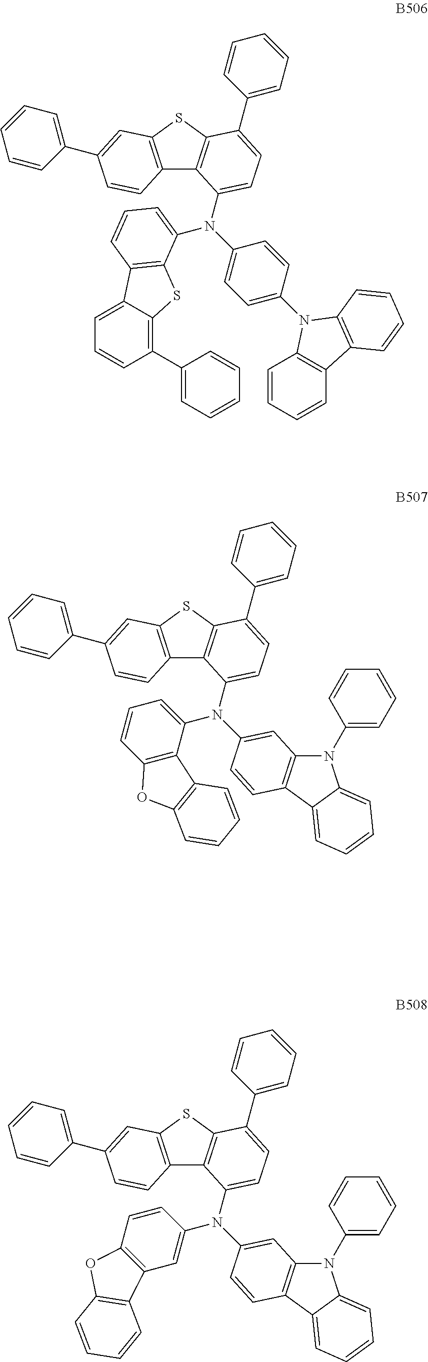 [SMILES: c1ccc(-c2ccc3c(c2)sc2c(-c4ccccc4)ccc(N(c4ccc(-n5c6ccccc6c6ccccc65)cc4)c4cccc5c4sc4c(-c6ccccc6)cccc45)c23)cc1 ; c1ccc(-c2ccc3c(c2)sc2c(-c4ccccc4)ccc(N(c4ccc5c6ccccc6n(-c6ccccc6)c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4ccc5c6ccccc6n(-c6ccccc6)c5c4)c23)cc1]

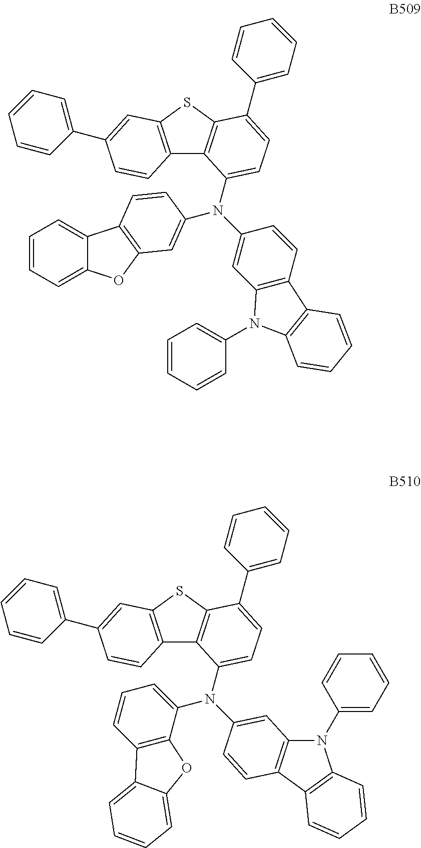 [SMILES: c1ccc(-c2ccc3c(c2)sc2c(-c4ccccc4)ccc(N(c4ccc5c(c4)oc4ccccc45)c4ccc5c6ccccc6n(-c6ccccc6)c5c4)c23)cc1 ; c1ccc(-c2ccc3c(c2)sc2c(-c4ccccc4)ccc(N(c4ccc5c6ccccc6n(-c6ccccc6)c5c4)c4cccc5c4oc4ccccc45)c23)cc1]

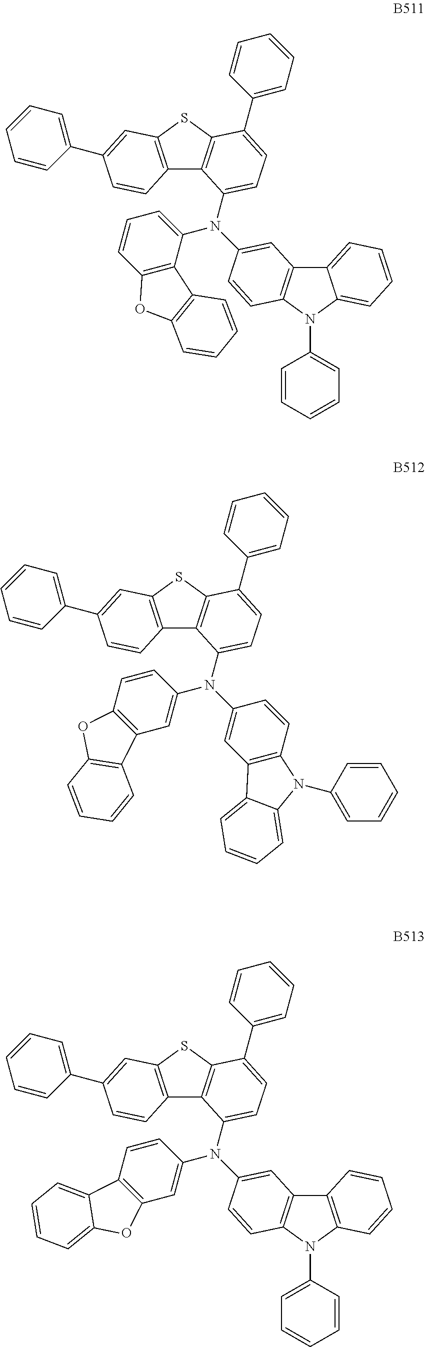 [SMILES: c1ccc(-c2ccc3c(c2)sc2c(-c4ccccc4)ccc(N(c4ccc5c(c4)c4ccccc4n5-c4ccccc4)c4cccc5oc6ccccc6c45)c23)cc1 ; c1ccc(-c2ccc3c(c2)sc2c(-c4ccccc4)ccc(N(c4ccc5oc6ccccc6c5c4)c4ccc5c(c4)c4ccccc4n5-c4ccccc4)c23)cc1 ; c1ccc(-c2ccc3c(c2)sc2c(-c4ccccc4)ccc(N(c4ccc5c(c4)oc4ccccc45)c4ccc5c(c4)c4ccccc4n5-c4ccccc4)c23)cc1]

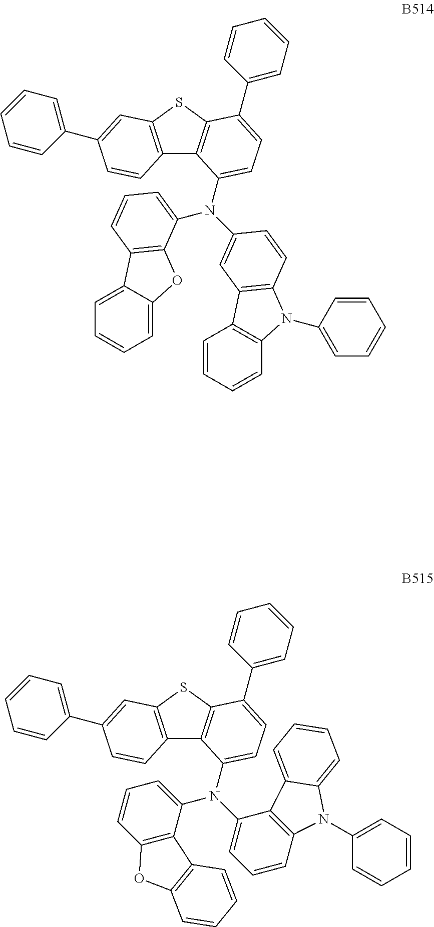 [SMILES: c1ccc(-c2ccc3c(c2)sc2c(-c4ccccc4)ccc(N(c4ccc5c(c4)c4ccccc4n5-c4ccccc4)c4cccc5c4oc4ccccc45)c23)cc1 ; c1ccc(-c2ccc3c(c2)sc2c(-c4ccccc4)ccc(N(c4cccc5oc6ccccc6c45)c4cccc5c4c4ccccc4n5-c4ccccc4)c23)cc1]

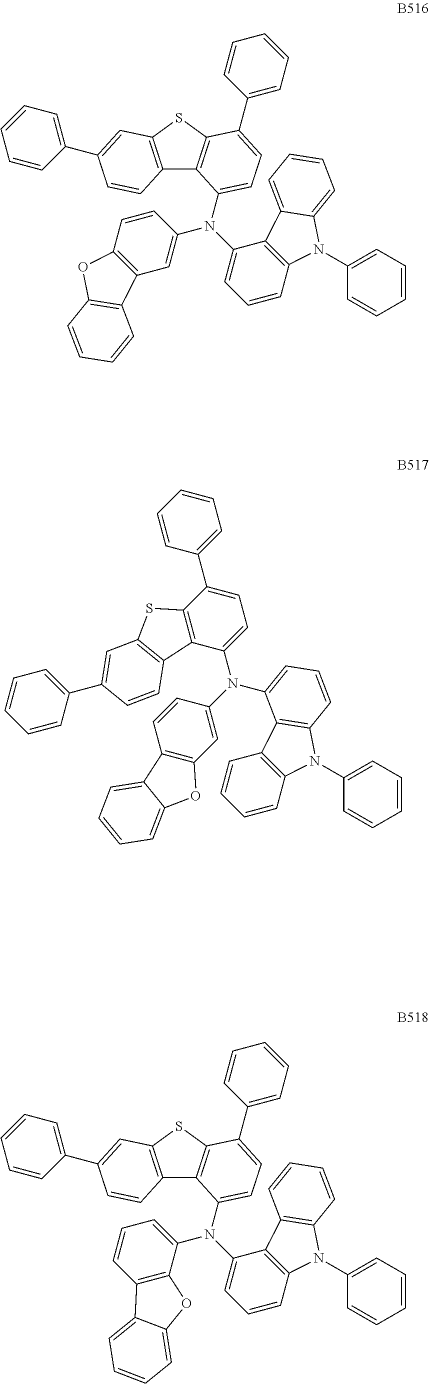 [SMILES: c1ccc(-c2ccc3c(c2)sc2c(-c4ccccc4)ccc(N(c4ccc5oc6ccccc6c5c4)c4cccc5c4c4ccccc4n5-c4ccccc4)c23)cc1 ; c1ccc(-c2ccc3c(c2)sc2c(-c4ccccc4)ccc(N(c4ccc5c(c4)oc4ccccc45)c4cccc5c4c4ccccc4n5-c4ccccc4)c23)cc1 ; c1ccc(-c2ccc3c(c2)sc2c(-c4ccccc4)ccc(N(c4cccc5c4oc4ccccc45)c4cccc5c4c4ccccc4n5-c4ccccc4)c23)cc1]

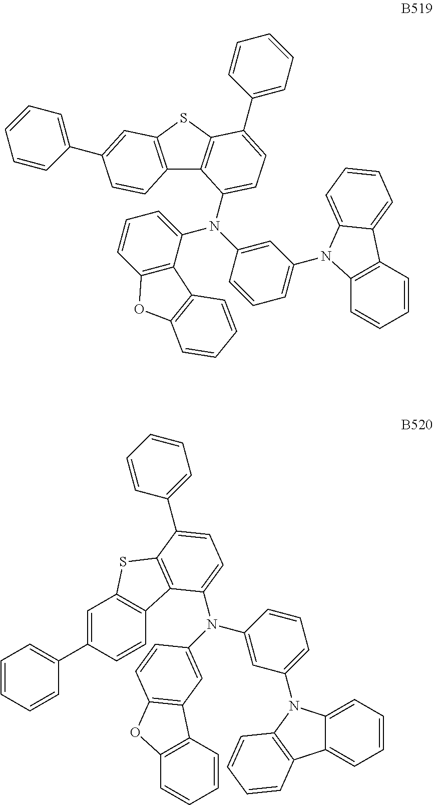 [SMILES: c1ccc(-c2ccc3c(c2)sc2c(-c4ccccc4)ccc(N(c4cccc(-n5c6ccccc6c6ccccc65)c4)c4cccc5oc6ccccc6c45)c23)cc1 ; c1ccc(-c2ccc3c(c2)sc2c(-c4ccccc4)ccc(N(c4cccc(-n5c6ccccc6c6ccccc65)c4)c4ccc5oc6ccccc6c5c4)c23)cc1]

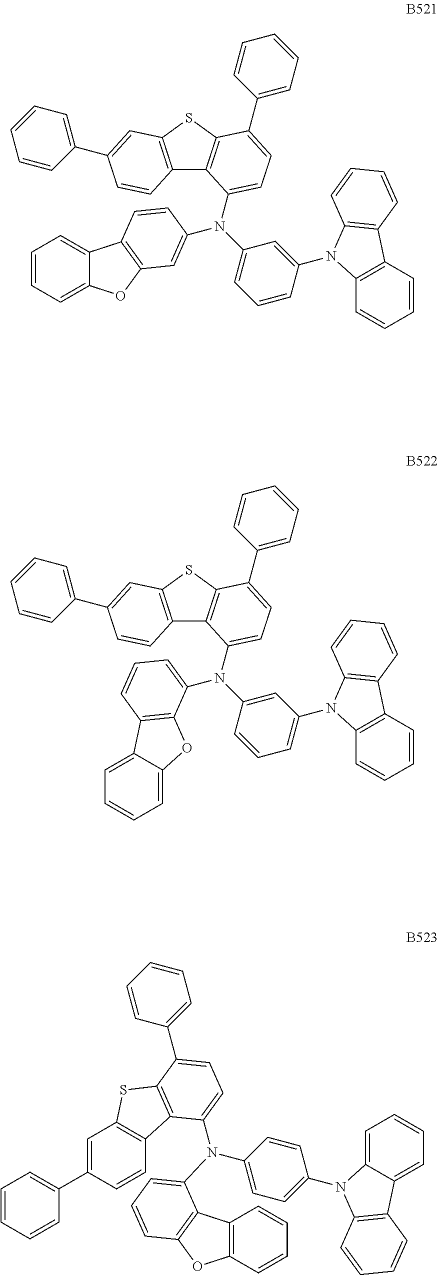 [SMILES: c1ccc(-c2ccc3c(c2)sc2c(-c4ccccc4)ccc(N(c4cccc(-n5c6ccccc6c6ccccc65)c4)c4ccc5c(c4)oc4ccccc45)c23)cc1 ; c1ccc(-c2ccc3c(c2)sc2c(-c4ccccc4)ccc(N(c4cccc(-n5c6ccccc6c6ccccc65)c4)c4cccc5c4oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5oc6ccccc6c45)c23)cc1]

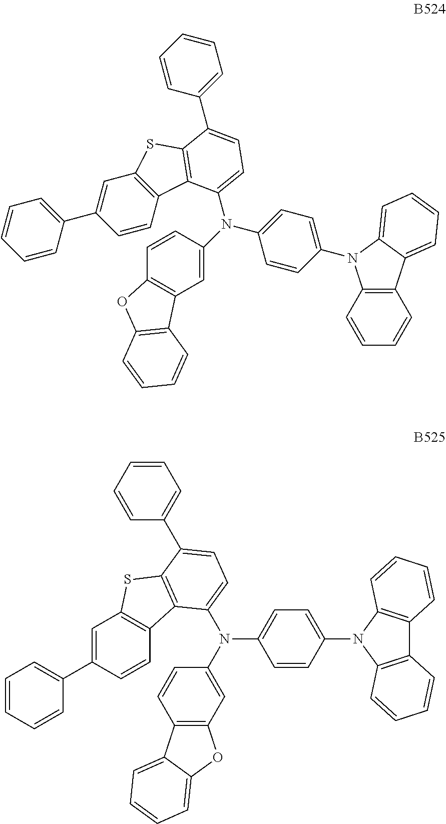 [SMILES: c1ccc(-c2ccc3c(c2)sc2c(-c4ccccc4)ccc(N(c4ccc(-n5c6ccccc6c6ccccc65)cc4)c4ccc5oc6ccccc6c5c4)c23)cc1 ; c1ccc(-c2ccc3c(c2)sc2c(-c4ccccc4)ccc(N(c4ccc(-n5c6ccccc6c6ccccc65)cc4)c4ccc5c(c4)oc4ccccc45)c23)cc1]

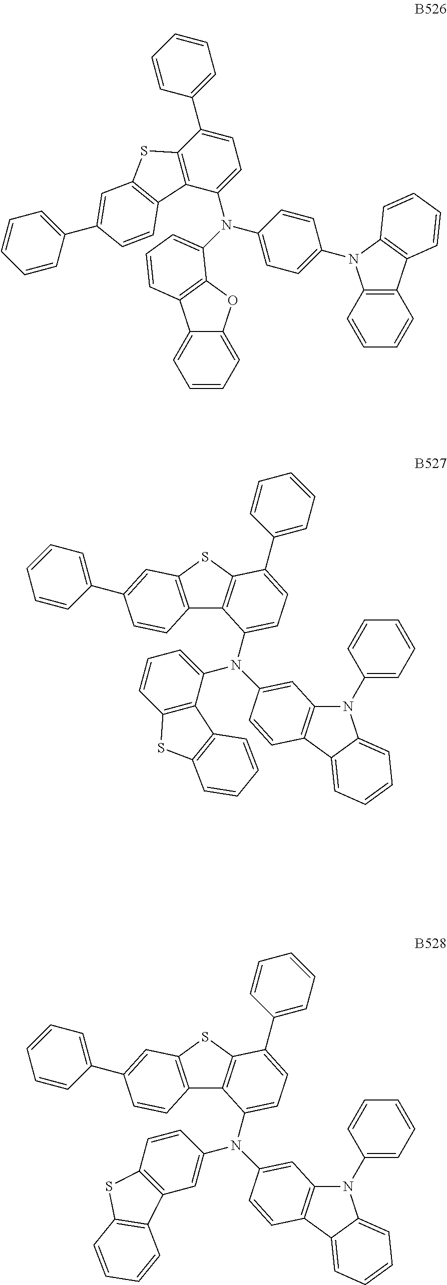 [SMILES: c1ccc(-c2ccc3c(c2)sc2c(-c4ccccc4)ccc(N(c4ccc(-n5c6ccccc6c6ccccc65)cc4)c4cccc5c4oc4ccccc45)c23)cc1 ; c1ccc(-c2ccc3c(c2)sc2c(-c4ccccc4)ccc(N(c4ccc5c6ccccc6n(-c6ccccc6)c5c4)c4cccc5sc6ccccc6c45)c23)cc1 ; c1ccc(-c2ccc3c(c2)sc2c(-c4ccccc4)ccc(N(c4ccc5sc6ccccc6c5c4)c4ccc5c6ccccc6n(-c6ccccc6)c5c4)c23)cc1]

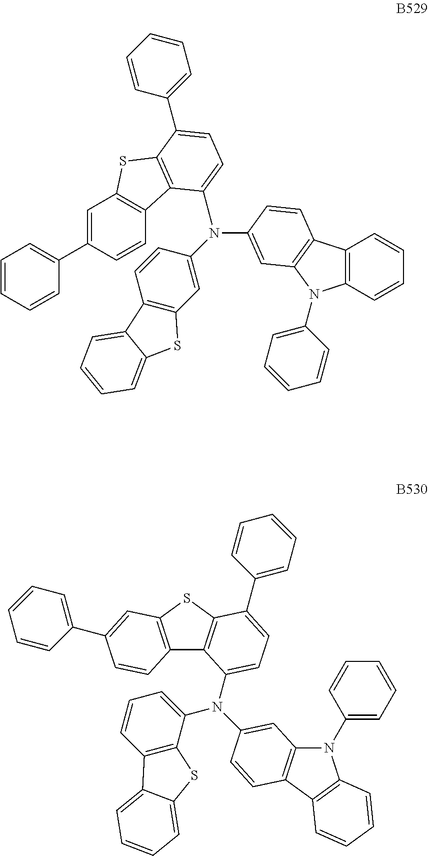 [SMILES: c1ccc(-c2ccc3c(c2)sc2c(-c4ccccc4)ccc(N(c4ccc5c(c4)sc4ccccc45)c4ccc5c6ccccc6n(-c6ccccc6)c5c4)c23)cc1 ; c1ccc(-c2ccc3c(c2)sc2c(-c4ccccc4)ccc(N(c4ccc5c6ccccc6n(-c6ccccc6)c5c4)c4cccc5c4sc4ccccc45)c23)cc1]

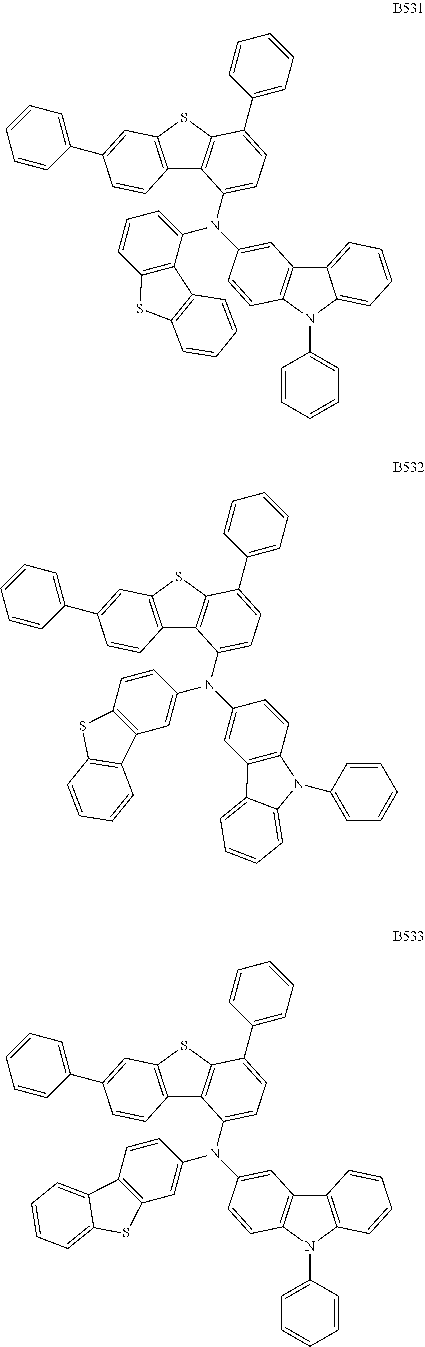 [SMILES: c1ccc(-c2ccc3c(c2)sc2c(-c4ccccc4)ccc(N(c4ccc5c(c4)c4ccccc4n5-c4ccccc4)c4cccc5sc6ccccc6c45)c23)cc1 ; c1ccc(-c2ccc3c(c2)sc2c(-c4ccccc4)ccc(N(c4ccc5sc6ccccc6c5c4)c4ccc5c(c4)c4ccccc4n5-c4ccccc4)c23)cc1 ; c1ccc(-c2ccc3c(c2)sc2c(-c4ccccc4)ccc(N(c4ccc5c(c4)sc4ccccc45)c4ccc5c(c4)c4ccccc4n5-c4ccccc4)c23)cc1]

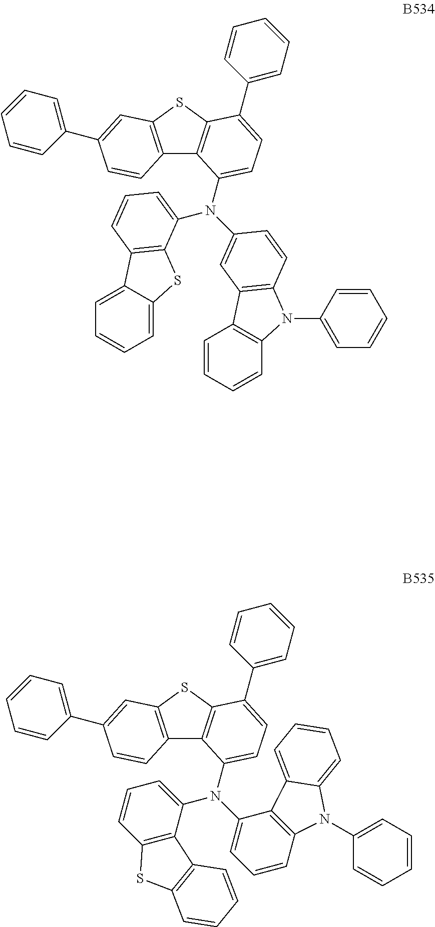 [SMILES: c1ccc(-c2ccc3c(c2)sc2c(-c4ccccc4)ccc(N(c4ccc5c(c4)c4ccccc4n5-c4ccccc4)c4cccc5c4sc4ccccc45)c23)cc1 ; c1ccc(-c2ccc3c(c2)sc2c(-c4ccccc4)ccc(N(c4cccc5sc6ccccc6c45)c4cccc5c4c4ccccc4n5-c4ccccc4)c23)cc1]

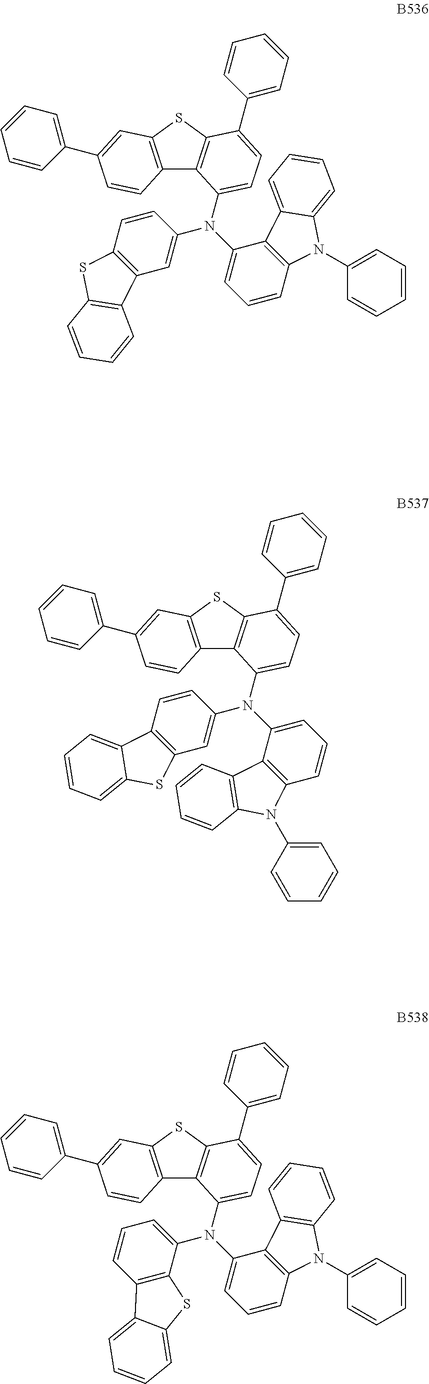 [SMILES: c1ccc(-c2ccc3c(c2)sc2c(-c4ccccc4)ccc(N(c4ccc5sc6ccccc6c5c4)c4cccc5c4c4ccccc4n5-c4ccccc4)c23)cc1 ; c1ccc(-c2ccc3c(c2)sc2c(-c4ccccc4)ccc(N(c4ccc5c(c4)sc4ccccc45)c4cccc5c4c4ccccc4n5-c4ccccc4)c23)cc1 ; c1ccc(-c2ccc3c(c2)sc2c(-c4ccccc4)ccc(N(c4cccc5c4sc4ccccc45)c4cccc5c4c4ccccc4n5-c4ccccc4)c23)cc1]

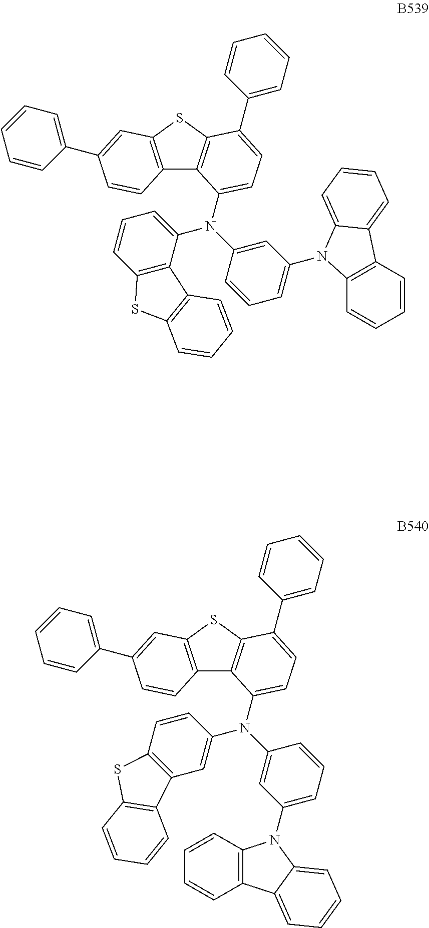 [SMILES: c1ccc(-c2ccc3c(c2)sc2c(-c4ccccc4)ccc(N(c4cccc(-n5c6ccccc6c6ccccc65)c4)c4cccc5sc6ccccc6c45)c23)cc1 ; c1ccc(-c2ccc3c(c2)sc2c(-c4ccccc4)ccc(N(c4cccc(-n5c6ccccc6c6ccccc65)c4)c4ccc5sc6ccccc6c5c4)c23)cc1]

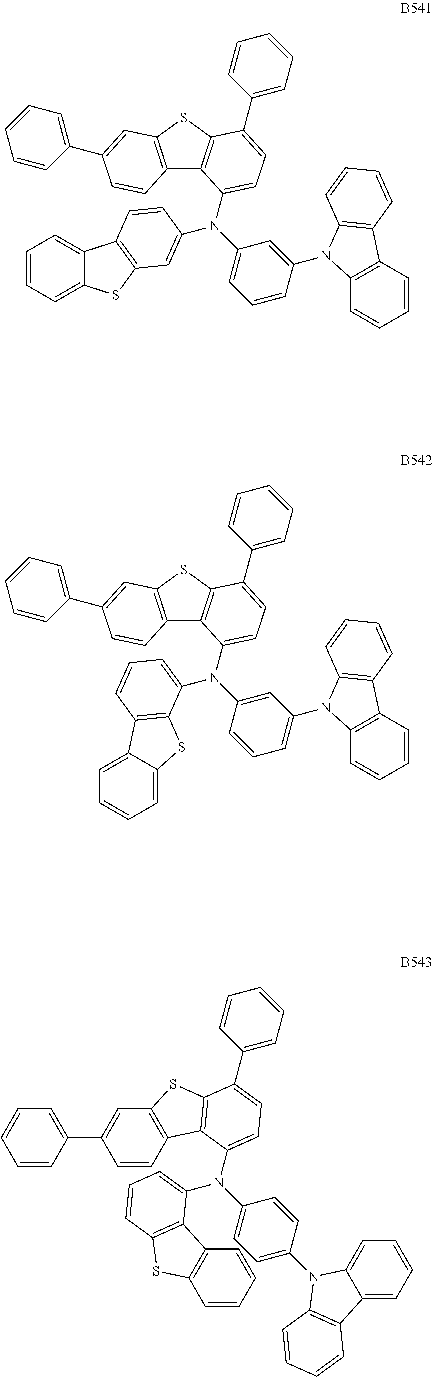 [SMILES: c1ccc(-c2ccc3c(c2)sc2c(-c4ccccc4)ccc(N(c4cccc(-n5c6ccccc6c6ccccc65)c4)c4ccc5c(c4)sc4ccccc45)c23)cc1 ; c1ccc(-c2ccc3c(c2)sc2c(-c4ccccc4)ccc(N(c4cccc(-n5c6ccccc6c6ccccc65)c4)c4cccc5c4sc4ccccc45)c23)cc1 ; c1ccc(-c2ccc3c(c2)sc2c(-c4ccccc4)ccc(N(c4ccc(-n5c6ccccc6c6ccccc65)cc4)c4cccc5sc6ccccc6c45)c23)cc1]

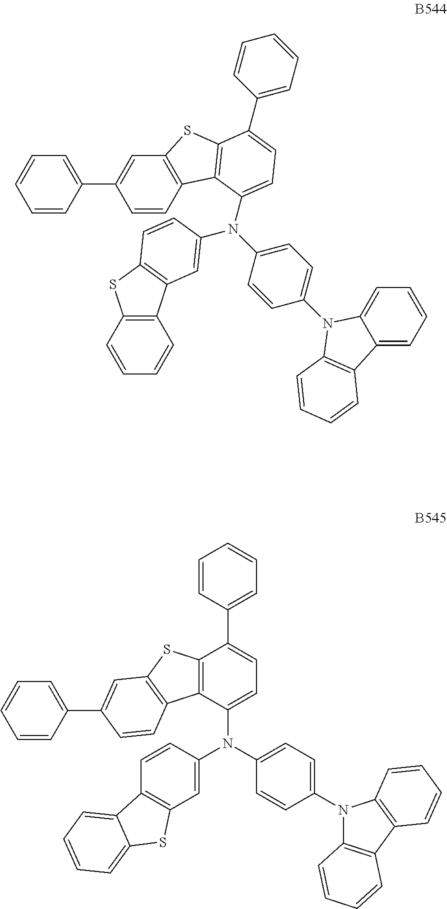 [SMILES: c1ccc(-c2ccc3c(c2)sc2c(-c4ccccc4)ccc(N(c4ccc(-n5c6ccccc6c6ccccc65)cc4)c4ccc5sc6ccccc6c5c4)c23)cc1 ; c1ccc(-c2ccc3c(c2)sc2c(-c4ccccc4)ccc(N(c4ccc(-n5c6ccccc6c6ccccc65)cc4)c4ccc5c(c4)sc4ccccc45)c23)cc1]

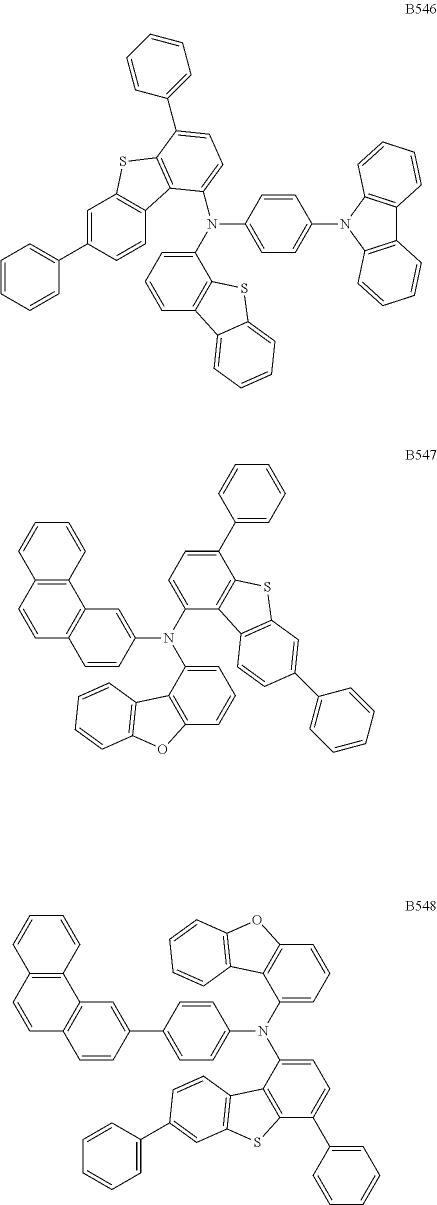 [SMILES: c1ccc(-c2ccc3c(c2)sc2c(-c4ccccc4)ccc(N(c4ccc(-n5c6ccccc6c6ccccc65)cc4)c4cccc5c4sc4ccccc45)c23)cc1 ; c1ccc(-c2ccc3c(c2)sc2c(-c4ccccc4)ccc(N(c4ccc5ccc6ccccc6c5c4)c4cccc5oc6ccccc6c45)c23)cc1 ; c1ccc(-c2ccc3c(c2)sc2c(-c4ccccc4)ccc(N(c4ccc(-c5ccc6ccc7ccccc7c6c5)cc4)c4cccc5oc6ccccc6c45)c23)cc1]

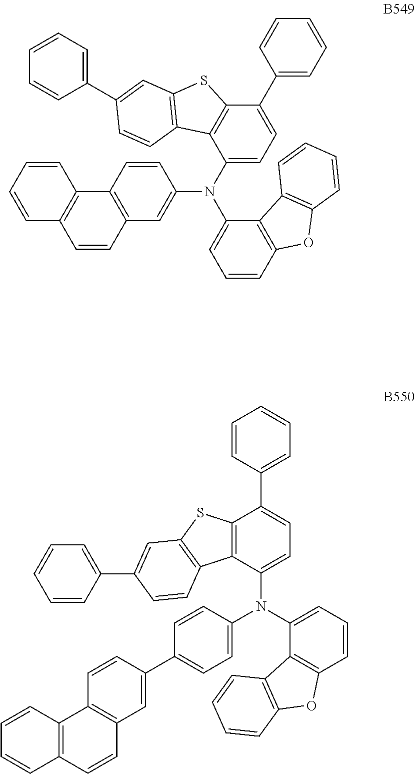 [SMILES: c1ccc(-c2ccc3c(c2)sc2c(-c4ccccc4)ccc(N(c4ccc5c(ccc6ccccc65)c4)c4cccc5oc6ccccc6c45)c23)cc1 ; c1ccc(-c2ccc3c(c2)sc2c(-c4ccccc4)ccc(N(c4ccc(-c5ccc6c(ccc7ccccc76)c5)cc4)c4cccc5oc6ccccc6c45)c23)cc1]

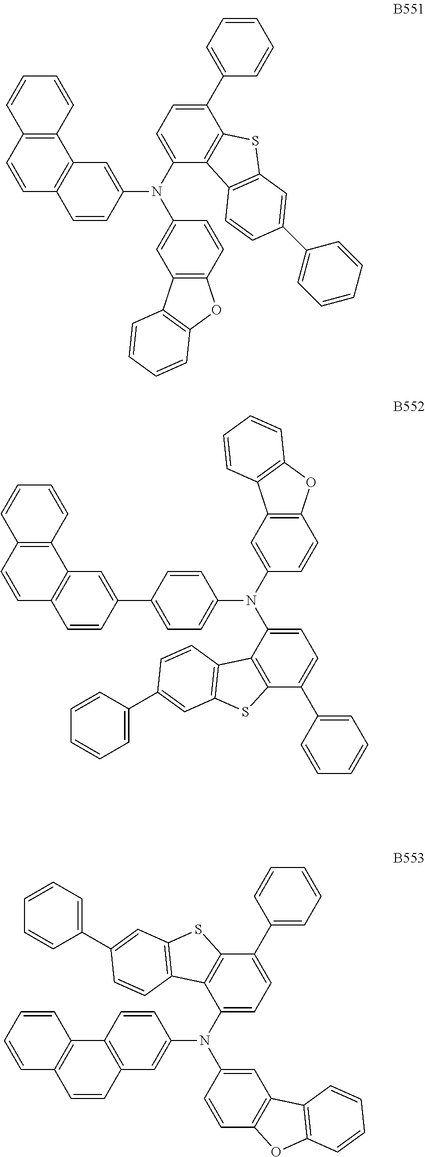 [SMILES: c1ccc(-c2ccc3c(c2)sc2c(-c4ccccc4)ccc(N(c4ccc5ccc6ccccc6c5c4)c4ccc5oc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5oc6ccccc6c5c4)c23)cc1 ; c1ccc(-c2ccc3c(c2)sc2c(-c4ccccc4)ccc(N(c4ccc5c(ccc6ccccc65)c4)c4ccc5oc6ccccc6c5c4)c23)cc1]

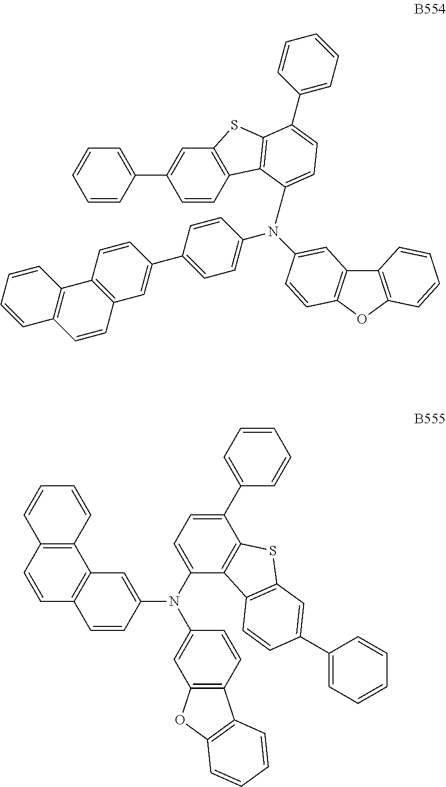 [SMILES: c1ccc(-c2ccc3c(c2)sc2c(-c4ccccc4)ccc(N(c4ccc(-c5ccc6c(ccc7ccccc76)c5)cc4)c4ccc5oc6ccccc6c5c4)c23)cc1 ; c1ccc(-c2ccc3c(c2)sc2c(-c4ccccc4)ccc(N(c4ccc5c(c4)oc4ccccc45)c4ccc5ccc6ccccc6c5c4)c23)cc1]

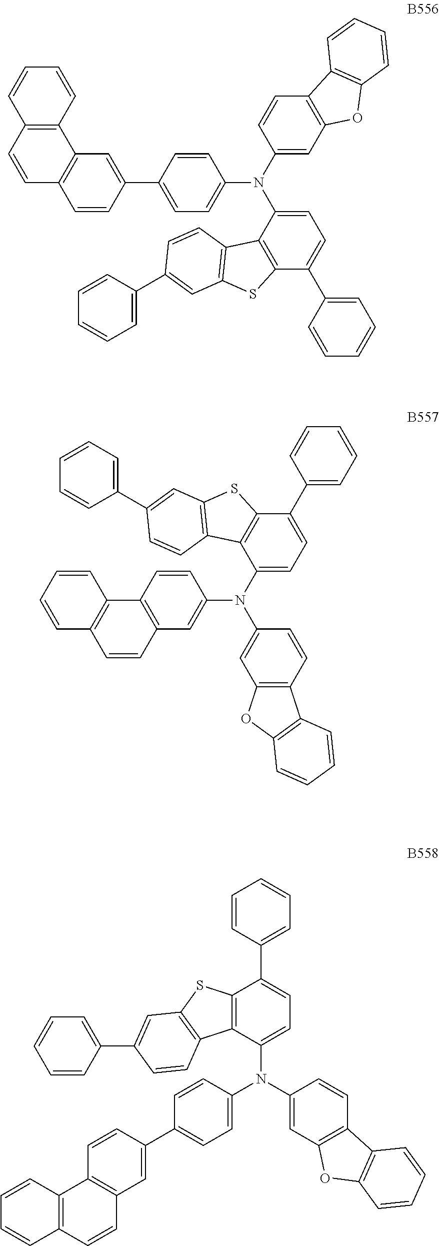 [SMILES: c1ccc(-c2ccc3c(c2)sc2c(-c4ccccc4)ccc(N(c4ccc(-c5ccc6ccc7ccccc7c6c5)cc4)c4ccc5c(c4)oc4ccccc45)c23)cc1 ; c1ccc(-c2ccc3c(c2)sc2c(-c4ccccc4)ccc(N(c4ccc5c(ccc6ccccc65)c4)c4ccc5c(c4)oc4ccccc45)c23)cc1 ; c1ccc(-c2ccc3c(c2)sc2c(-c4ccccc4)ccc(N(c4ccc(-c5ccc6c(ccc7ccccc76)c5)cc4)c4ccc5c(c4)oc4ccccc45)c23)cc1]

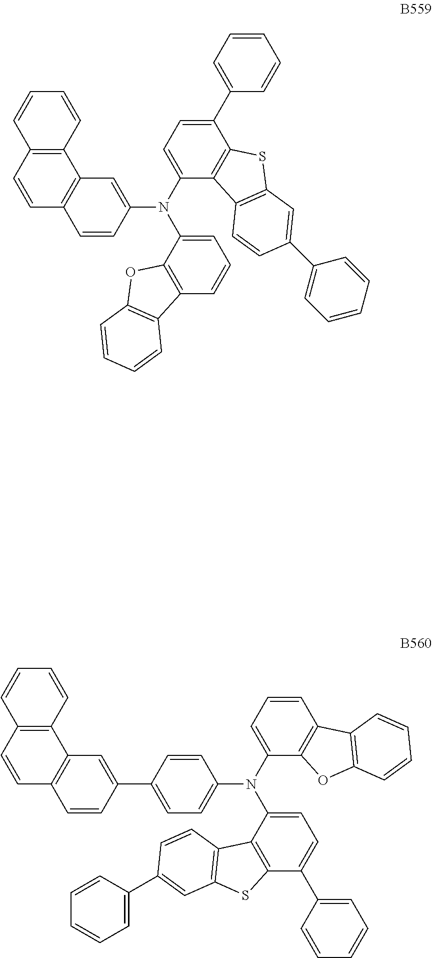 [SMILES: c1ccc(-c2ccc3c(c2)sc2c(-c4ccccc4)ccc(N(c4ccc5ccc6ccccc6c5c4)c4cccc5c4oc4ccccc45)c23)cc1 ; c1ccc(-c2ccc3c(c2)sc2c(-c4ccccc4)ccc(N(c4ccc(-c5ccc6ccc7ccccc7c6c5)cc4)c4cccc5c4oc4ccccc45)c23)cc1]

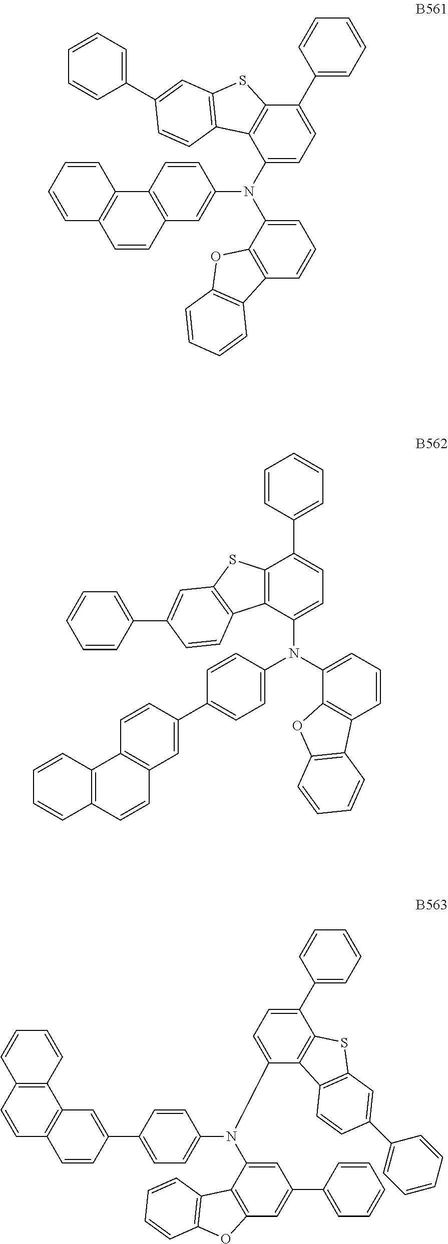 [SMILES: c1ccc(-c2ccc3c(c2)sc2c(-c4ccccc4)ccc(N(c4ccc5c(ccc6ccccc65)c4)c4cccc5c4oc4ccccc45)c23)cc1 ; c1ccc(-c2ccc3c(c2)sc2c(-c4ccccc4)ccc(N(c4ccc(-c5ccc6c(ccc7ccccc76)c5)cc4)c4cccc5c4oc4ccccc45)c23)cc1 ; c1ccc(-c2cc(N(c3ccc(-c4ccc5ccc6ccccc6c5c4)cc3)c3ccc(-c4ccccc4)c4sc5cc(-c6ccccc6)ccc5c34)c3c(c2)oc2ccccc23)cc1]

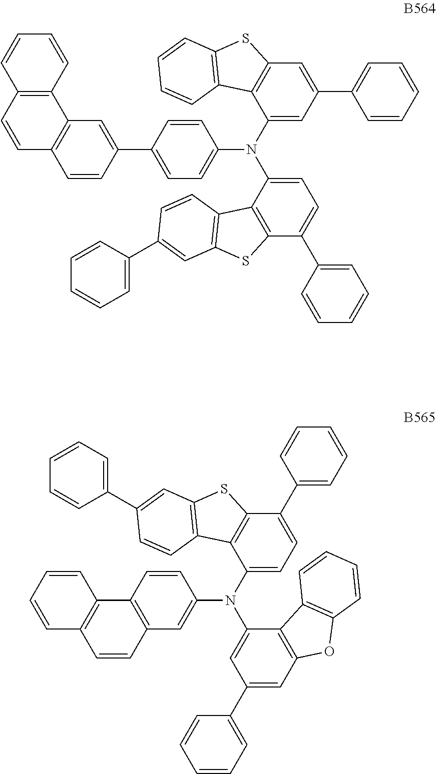 [SMILES: c1ccc(-c2ccc3c(c2)sc2c(-c4ccccc4)ccc(N(c4ccc(-c5ccc6ccc7ccccc7c6c5)cc4)c4cc(-c5ccccc5)cc5sc6ccccc6c45)c23)cc1 ; c1ccc(-c2cc(N(c3ccc4c(ccc5ccccc54)c3)c3ccc(-c4ccccc4)c4sc5cc(-c6ccccc6)ccc5c34)c3c(c2)oc2ccccc23)cc1]

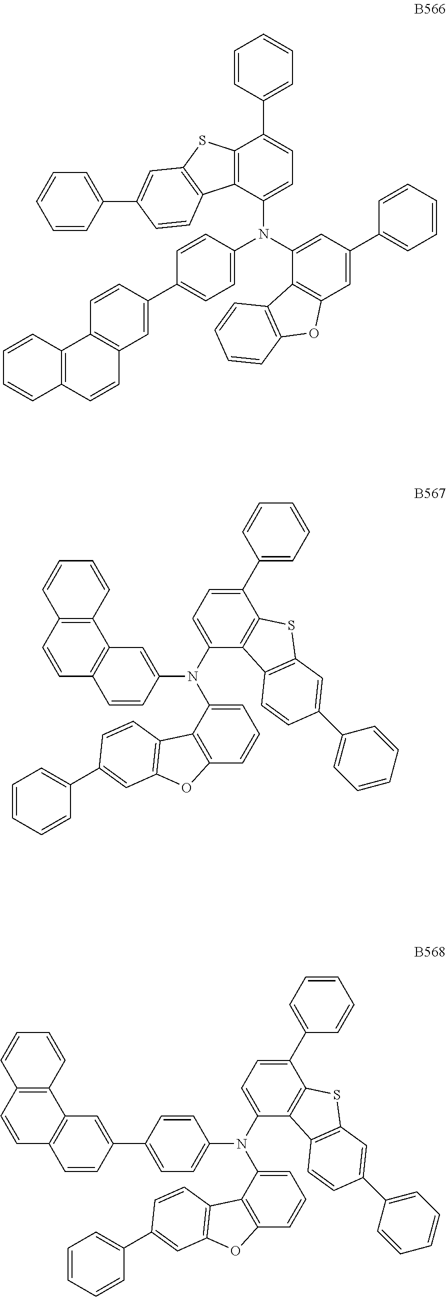 [SMILES: c1ccc(-c2cc(N(c3ccc(-c4ccc5c(ccc6ccccc65)c4)cc3)c3ccc(-c4ccccc4)c4sc5cc(-c6ccccc6)ccc5c34)c3c(c2)oc2ccccc23)cc1 ; c1ccc(-c2ccc3c(c2)oc2cccc(N(c4ccc5ccc6ccccc6c5c4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5sc6cc(-c7ccccc7)ccc6c45)c23)cc1]

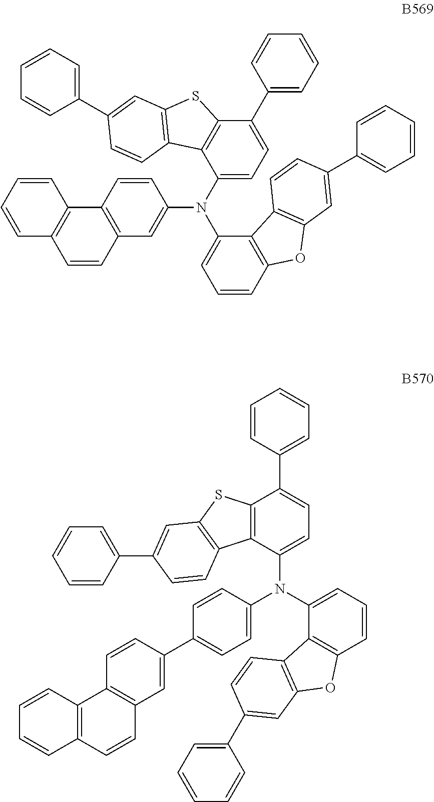 [SMILES: c1ccc(-c2ccc3c(c2)oc2cccc(N(c4ccc5c(ccc6ccccc65)c4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5sc6cc(-c7ccccc7)ccc6c45)c23)cc1]

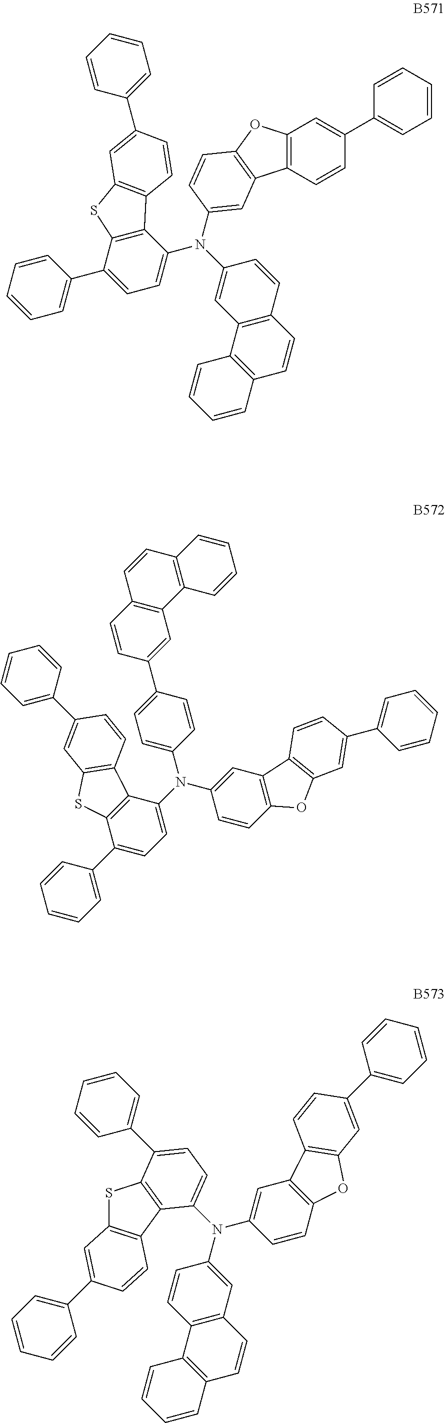 [SMILES: c1ccc(-c2ccc3c(c2)oc2ccc(N(c4ccc5ccc6ccccc6c5c4)c4ccc(-c5ccccc5)c5sc6cc(-c7ccccc7)ccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5sc6cc(-c7ccccc7)ccc6c45)cc23)cc1 ; c1ccc(-c2ccc3c(c2)oc2ccc(N(c4ccc5c(ccc6ccccc65)c4)c4ccc(-c5ccccc5)c5sc6cc(-c7ccccc7)ccc6c45)cc23)cc1]

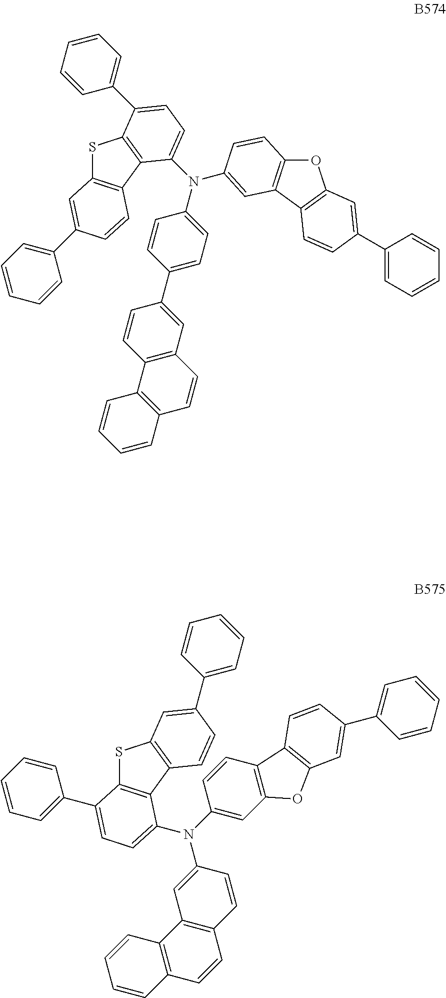 [SMILES: c1ccc(-c2ccc3c(c2)oc2ccc(N(c4ccc(-c5ccc6c(ccc7ccccc76)c5)cc4)c4ccc(-c5ccccc5)c5sc6cc(-c7ccccc7)ccc6c45)cc23)cc1 ; c1ccc(-c2ccc3c(c2)oc2cc(N(c4ccc5ccc6ccccc6c5c4)c4ccc(-c5ccccc5)c5sc6cc(-c7ccccc7)ccc6c45)ccc23)cc1]

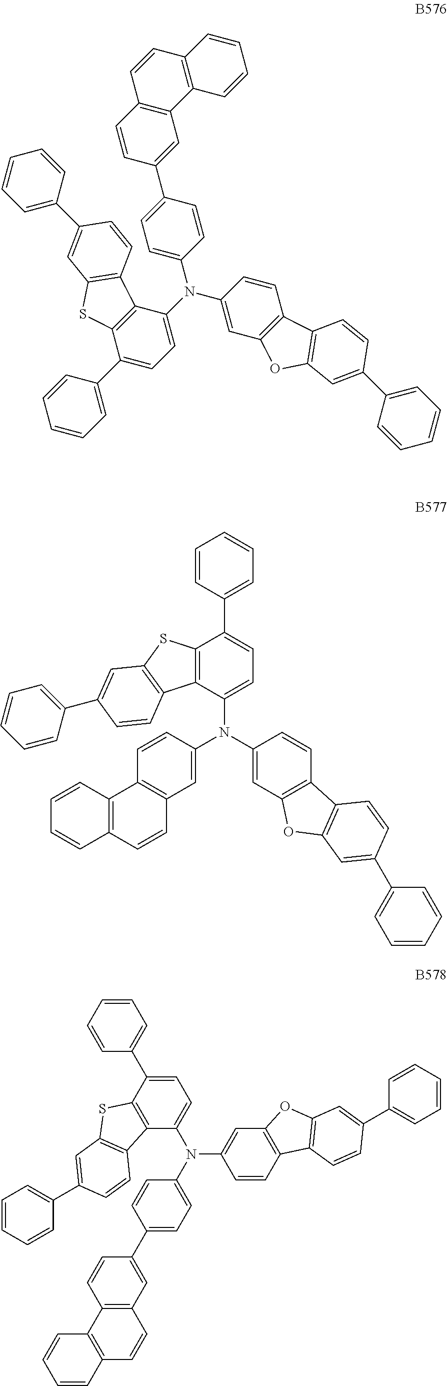 [SMILES: c1ccc(-c2ccc3c(c2)oc2cc(N(c4ccc(-c5ccc6ccc7ccccc7c6c5)cc4)c4ccc(-c5ccccc5)c5sc6cc(-c7ccccc7)ccc6c45)ccc23)cc1 ; c1ccc(-c2ccc3c(c2)oc2cc(N(c4ccc5c(ccc6ccccc65)c4)c4ccc(-c5ccccc5)c5sc6cc(-c7ccccc7)ccc6c45)ccc23)cc1 ; c1ccc(-c2ccc3c(c2)oc2cc(N(c4ccc(-c5ccc6c(ccc7ccccc76)c5)cc4)c4ccc(-c5ccccc5)c5sc6cc(-c7ccccc7)ccc6c45)ccc23)cc1]

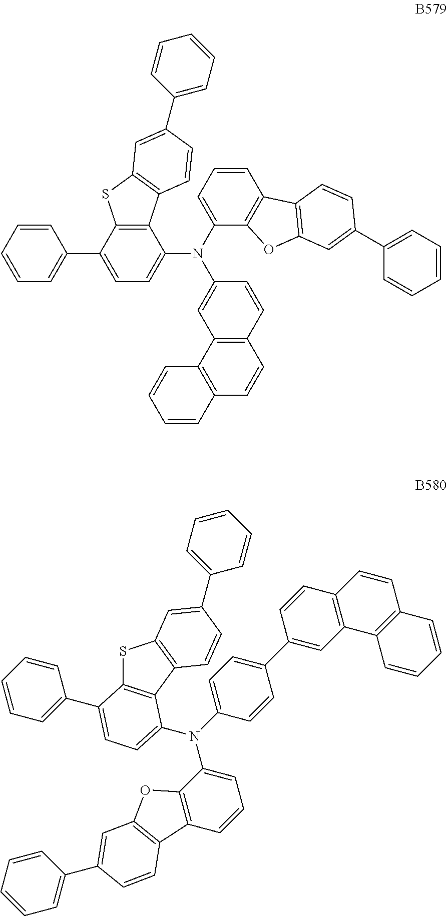 [SMILES: c1ccc(-c2ccc3c(c2)oc2c(N(c4ccc5ccc6ccccc6c5c4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5sc6cc(-c7ccccc7)ccc6c45)cccc23)cc1]

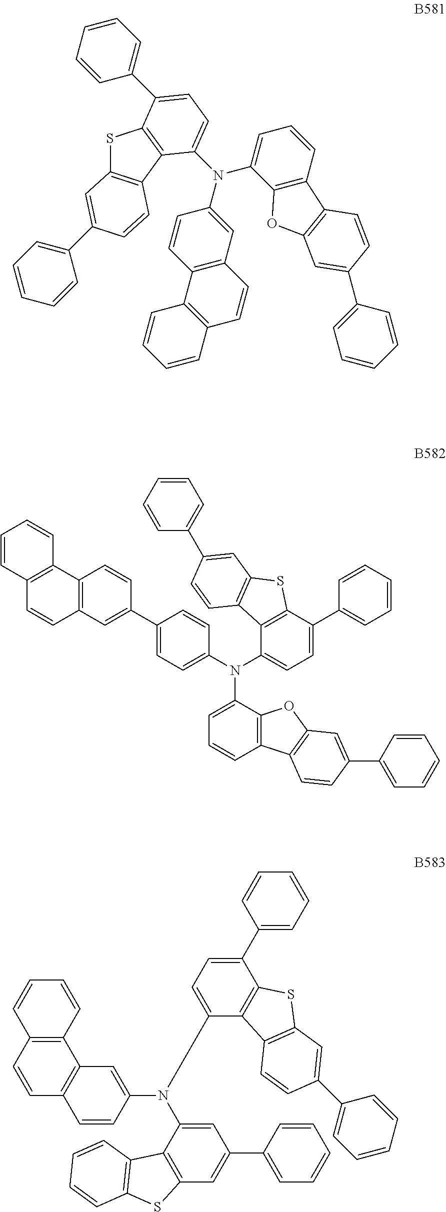 [SMILES: c1ccc(-c2ccc3c(c2)oc2c(N(c4ccc5c(ccc6ccccc65)c4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6c(ccc7ccccc76)c5)cc4)c4ccc(-c5ccccc5)c5sc6cc(-c7ccccc7)ccc6c45)cccc23)cc1 ; c1ccc(-c2ccc3c(c2)sc2c(-c4ccccc4)ccc(N(c4ccc5ccc6ccccc6c5c4)c4cc(-c5ccccc5)cc5sc6ccccc6c45)c23)cc1]

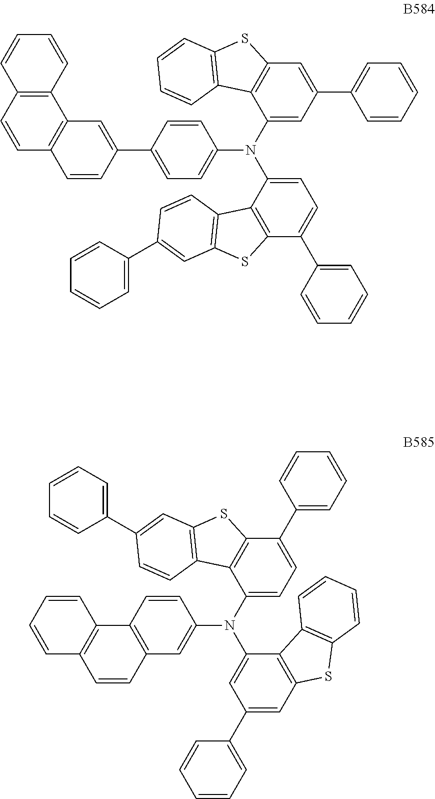 [SMILES: c1ccc(-c2ccc3c(c2)sc2c(-c4ccccc4)ccc(N(c4ccc(-c5ccc6ccc7ccccc7c6c5)cc4)c4cc(-c5ccccc5)cc5sc6ccccc6c45)c23)cc1 ; c1ccc(-c2ccc3c(c2)sc2c(-c4ccccc4)ccc(N(c4ccc5c(ccc6ccccc65)c4)c4cc(-c5ccccc5)cc5sc6ccccc6c45)c23)cc1]

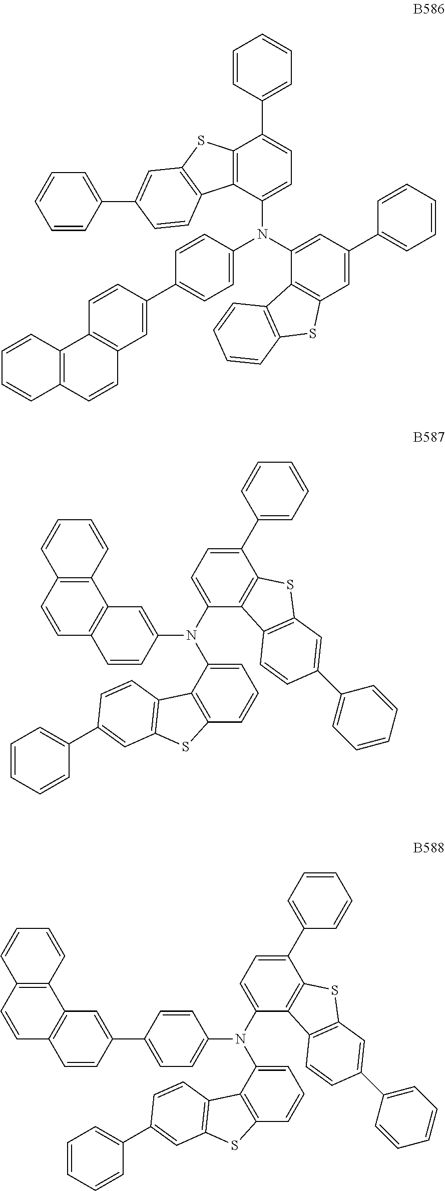 [SMILES: c1ccc(-c2ccc3c(c2)sc2c(-c4ccccc4)ccc(N(c4ccc(-c5ccc6c(ccc7ccccc76)c5)cc4)c4cc(-c5ccccc5)cc5sc6ccccc6c45)c23)cc1 ; c1ccc(-c2ccc3c(c2)sc2cccc(N(c4ccc5ccc6ccccc6c5c4)c4ccc(-c5ccccc5)c5sc6cc(-c7ccccc7)ccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5sc6cc(-c7ccccc7)ccc6c45)c23)cc1]

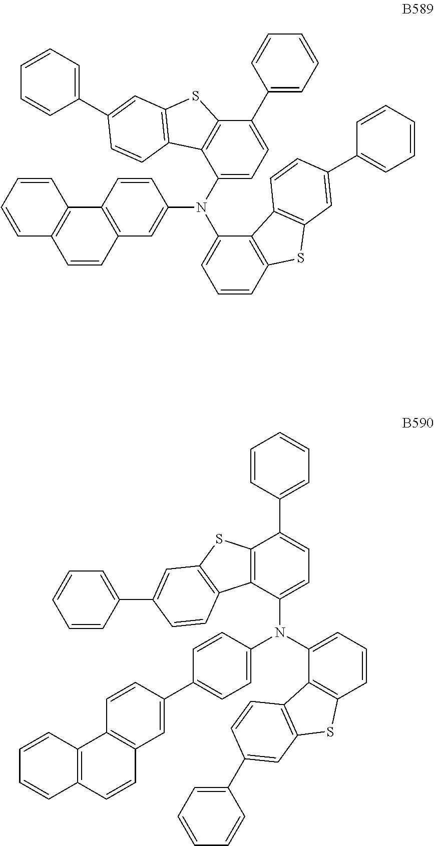 [SMILES: c1ccc(-c2ccc3c(c2)sc2cccc(N(c4ccc5c(ccc6ccccc65)c4)c4ccc(-c5ccccc5)c5sc6cc(-c7ccccc7)ccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5sc6cc(-c7ccccc7)ccc6c45)c23)cc1]

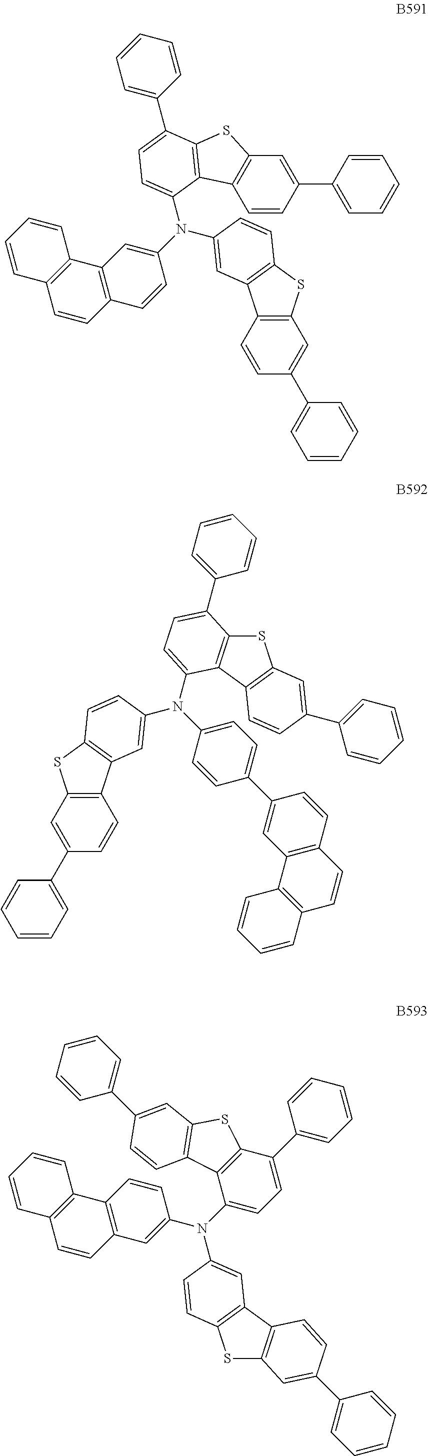 [SMILES: c1ccc(-c2ccc3c(c2)sc2ccc(N(c4ccc5ccc6ccccc6c5c4)c4ccc(-c5ccccc5)c5sc6cc(-c7ccccc7)ccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5sc6cc(-c7ccccc7)ccc6c45)cc23)cc1 ; c1ccc(-c2ccc3c(c2)sc2ccc(N(c4ccc5c(ccc6ccccc65)c4)c4ccc(-c5ccccc5)c5sc6cc(-c7ccccc7)ccc6c45)cc23)cc1]

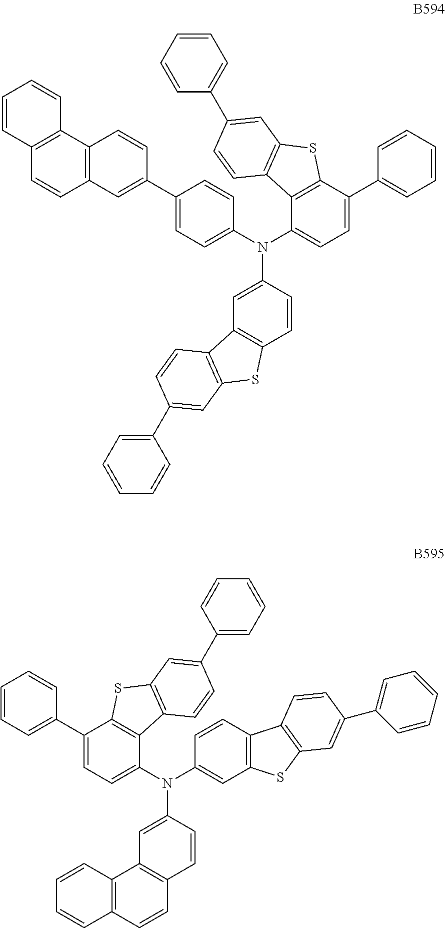 [SMILES: c1ccc(-c2ccc3c(c2)sc2ccc(N(c4ccc(-c5ccc6c(ccc7ccccc76)c5)cc4)c4ccc(-c5ccccc5)c5sc6cc(-c7ccccc7)ccc6c45)cc23)cc1 ; c1ccc(-c2ccc3c(c2)sc2cc(N(c4ccc5ccc6ccccc6c5c4)c4ccc(-c5ccccc5)c5sc6cc(-c7ccccc7)ccc6c45)ccc23)cc1]

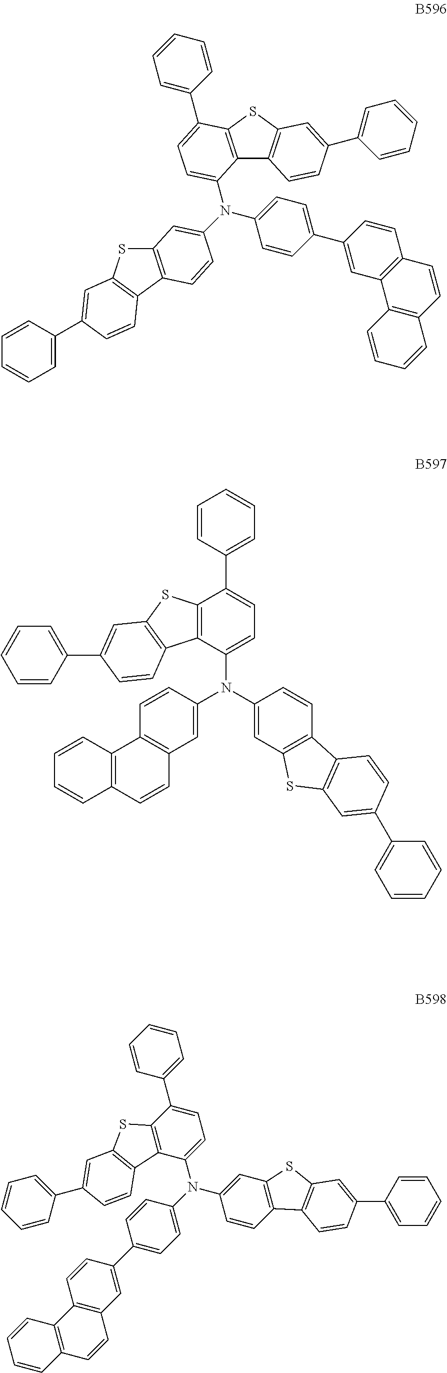 [SMILES: c1ccc(-c2ccc3c(c2)sc2cc(N(c4ccc(-c5ccc6ccc7ccccc7c6c5)cc4)c4ccc(-c5ccccc5)c5sc6cc(-c7ccccc7)ccc6c45)ccc23)cc1 ; c1ccc(-c2ccc3c(c2)sc2cc(N(c4ccc5c(ccc6ccccc65)c4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5sc6cc(-c7ccccc7)ccc6c45)ccc23)cc1]

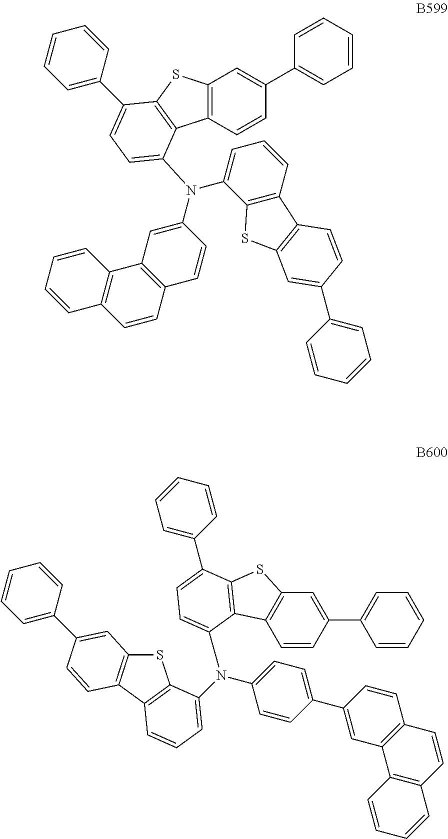 [SMILES: c1ccc(-c2ccc3c(c2)sc2c(N(c4ccc5ccc6ccccc6c5c4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5sc6cc(-c7ccccc7)ccc6c45)cccc23)cc1]

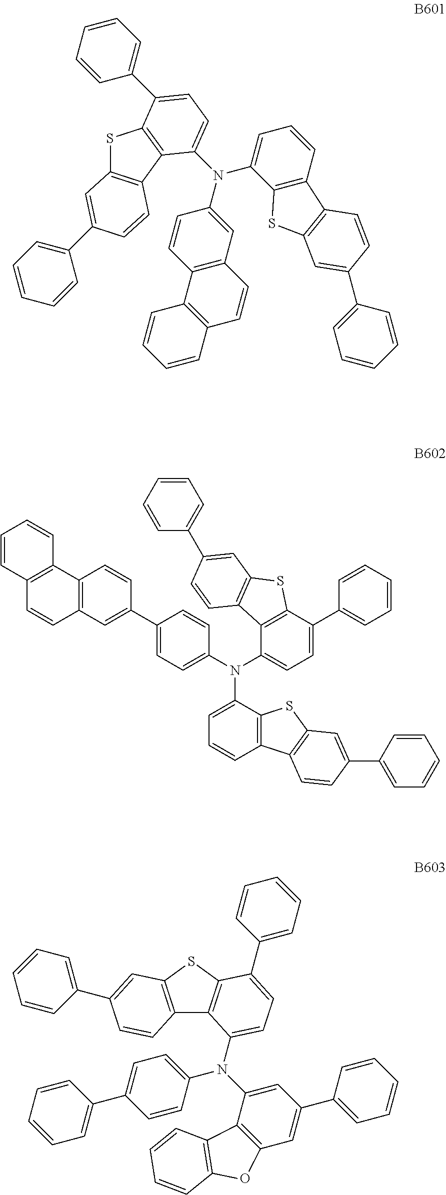 [SMILES: c1ccc(-c2ccc3c(c2)sc2c(N(c4ccc5c(ccc6ccccc65)c4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5sc6cc(-c7ccccc7)ccc6c45)cccc23)cc1 ; c1ccc(-c2ccc(N(c3cc(-c4ccccc4)cc4oc5ccccc5c34)c3ccc(-c4ccccc4)c4sc5cc(-c6ccccc6)ccc5c34)cc2)cc1]

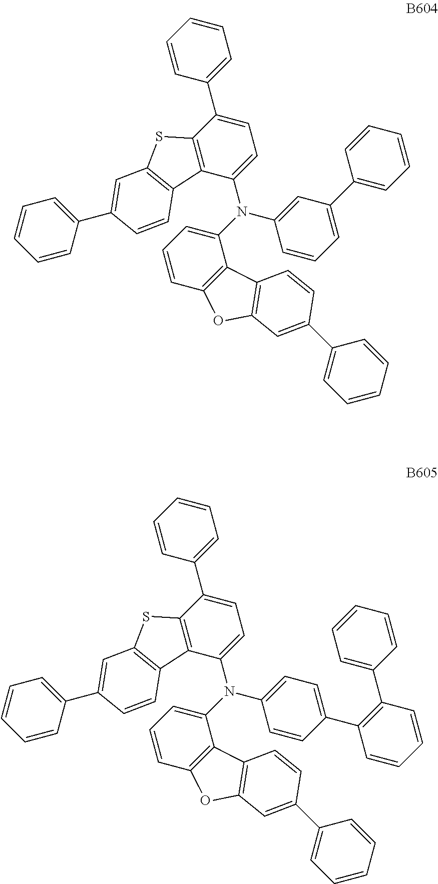 [SMILES: c1ccc(-c2cccc(N(c3cccc4oc5cc(-c6ccccc6)ccc5c34)c3ccc(-c4ccccc4)c4sc5cc(-c6ccccc6)ccc5c34)c2)cc1 ; c1ccc(-c2ccc3c(c2)oc2cccc(N(c4ccc(-c5ccccc5-c5ccccc5)cc4)c4ccc(-c5ccccc5)c5sc6cc(-c7ccccc7)ccc6c45)c23)cc1]

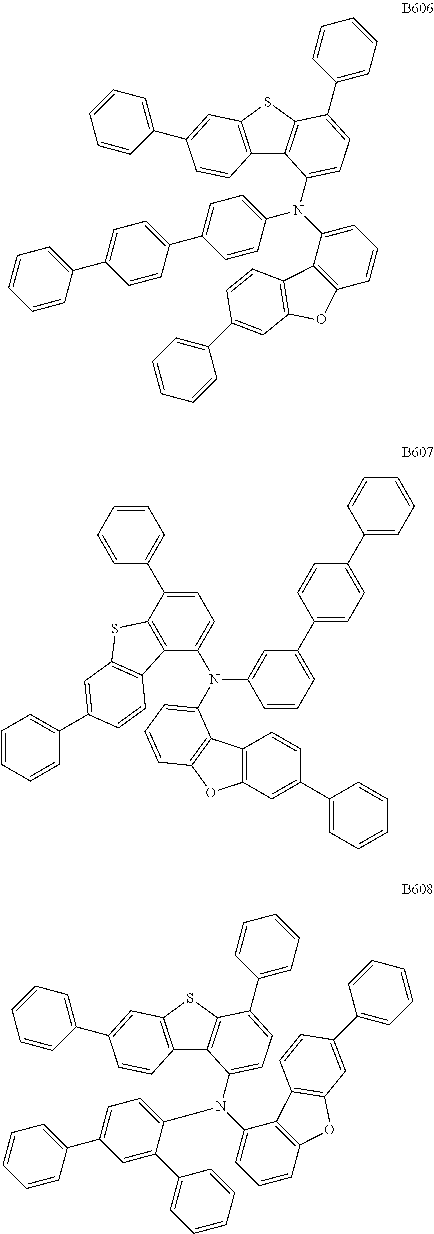 [SMILES: c1ccc(-c2ccc(-c3ccc(N(c4cccc5oc6cc(-c7ccccc7)ccc6c45)c4ccc(-c5ccccc5)c5sc6cc(-c7ccccc7)ccc6c45)cc3)cc2)cc1 ; c1ccc(-c2ccc(-c3cccc(N(c4cccc5oc6cc(-c7ccccc7)ccc6c45)c4ccc(-c5ccccc5)c5sc6cc(-c7ccccc7)ccc6c45)c3)cc2)cc1 ; c1ccc(-c2ccc(N(c3cccc4oc5cc(-c6ccccc6)ccc5c34)c3ccc(-c4ccccc4)c4sc5cc(-c6ccccc6)ccc5c34)c(-c3ccccc3)c2)cc1]

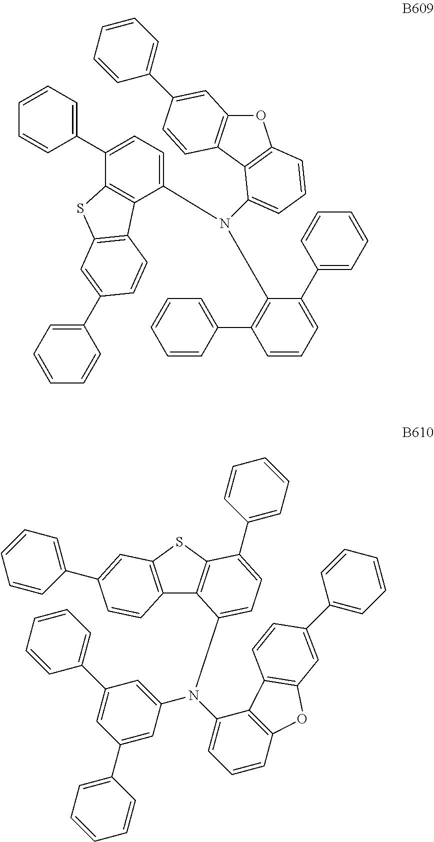 [SMILES: c1ccc(-c2ccc3c(c2)oc2cccc(N(c4c(-c5ccccc5)cccc4-c4ccccc4)c4ccc(-c5ccccc5)c5sc6cc(-c7ccccc7)ccc6c45)c23)cc1 ; c1ccc(-c2cc(-c3ccccc3)cc(N(c3cccc4oc5cc(-c6ccccc6)ccc5c34)c3ccc(-c4ccccc4)c4sc5cc(-c6ccccc6)ccc5c34)c2)cc1]

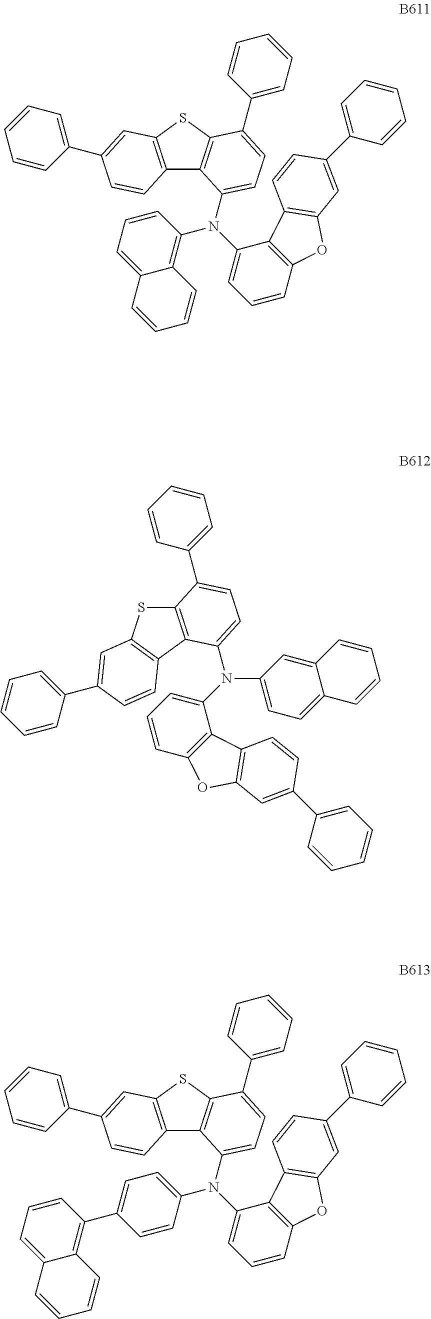 [SMILES: c1ccc(-c2ccc3c(c2)oc2cccc(N(c4cccc5ccccc45)c4ccc(-c5ccccc5)c5sc6cc(-c7ccccc7)ccc6c45)c23)cc1 ; c1ccc(-c2ccc3c(c2)oc2cccc(N(c4ccc5ccccc5c4)c4ccc(-c5ccccc5)c5sc6cc(-c7ccccc7)ccc6c45)c23)cc1 ; c1ccc(-c2ccc3c(c2)oc2cccc(N(c4ccc(-c5cccc6ccccc56)cc4)c4ccc(-c5ccccc5)c5sc6cc(-c7ccccc7)ccc6c45)c23)cc1]

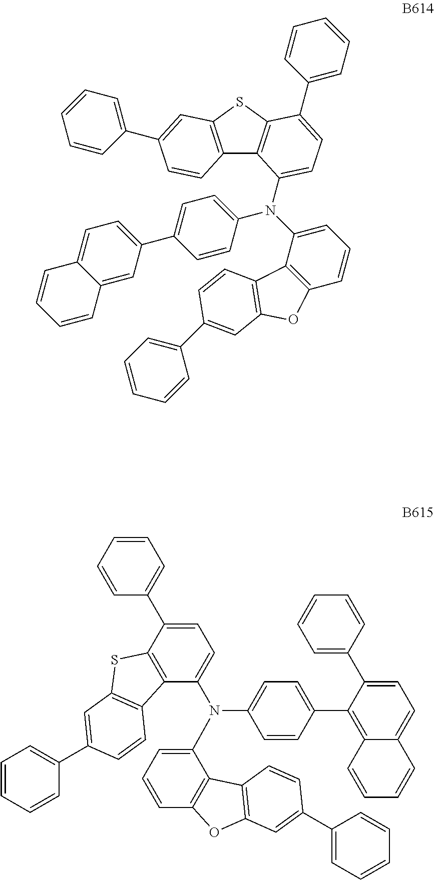 [SMILES: c1ccc(-c2ccc3c(c2)oc2cccc(N(c4ccc(-c5ccc6ccccc6c5)cc4)c4ccc(-c5ccccc5)c5sc6cc(-c7ccccc7)ccc6c45)c23)cc1 ; c1ccc(-c2ccc3c(c2)oc2cccc(N(c4ccc(-c5c(-c6ccccc6)ccc6ccccc56)cc4)c4ccc(-c5ccccc5)c5sc6cc(-c7ccccc7)ccc6c45)c23)cc1]

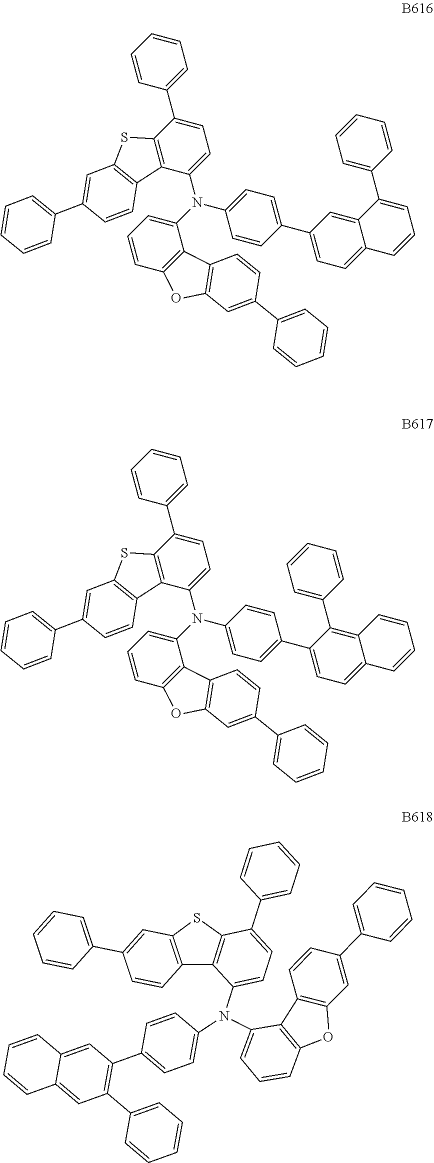 [SMILES: c1ccc(-c2ccc3c(c2)oc2cccc(N(c4ccc(-c5ccc6cccc(-c7ccccc7)c6c5)cc4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6ccccc6c5-c5ccccc5)cc4)c4ccc(-c5ccccc5)c5sc6cc(-c7ccccc7)ccc6c45)c23)cc1 ; c1ccc(-c2ccc3c(c2)oc2cccc(N(c4ccc(-c5cc6ccccc6cc5-c5ccccc5)cc4)c4ccc(-c5ccccc5)c5sc6cc(-c7ccccc7)ccc6c45)c23)cc1]

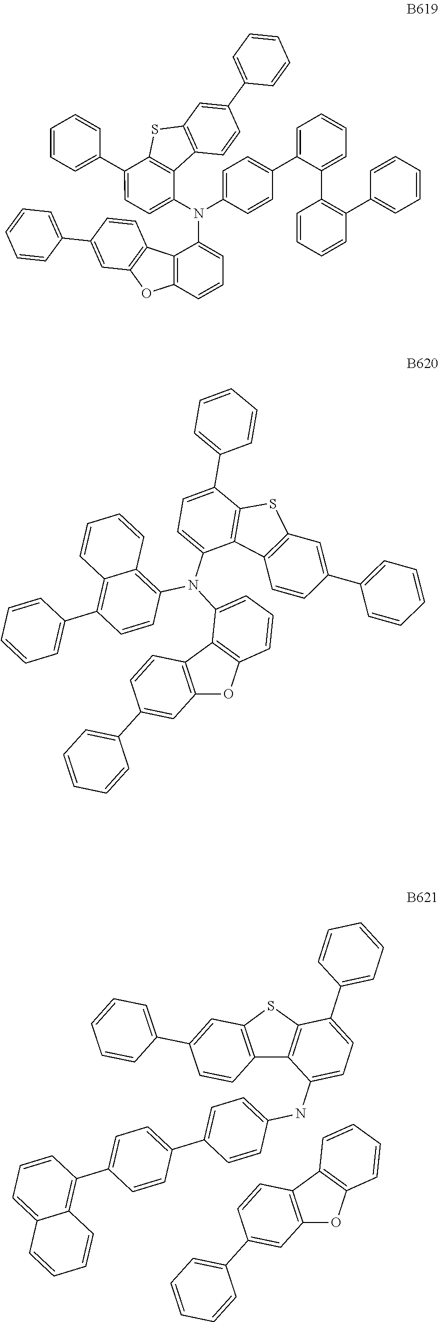

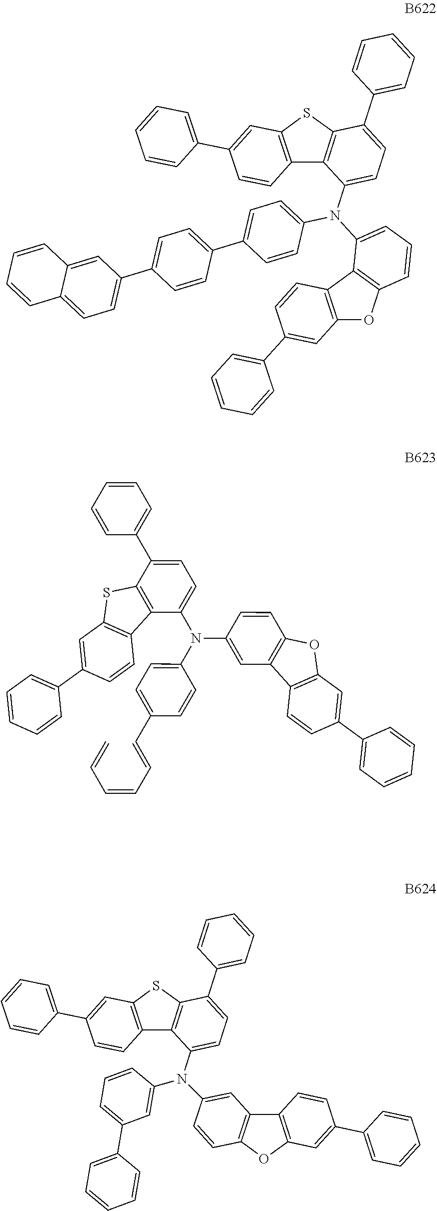

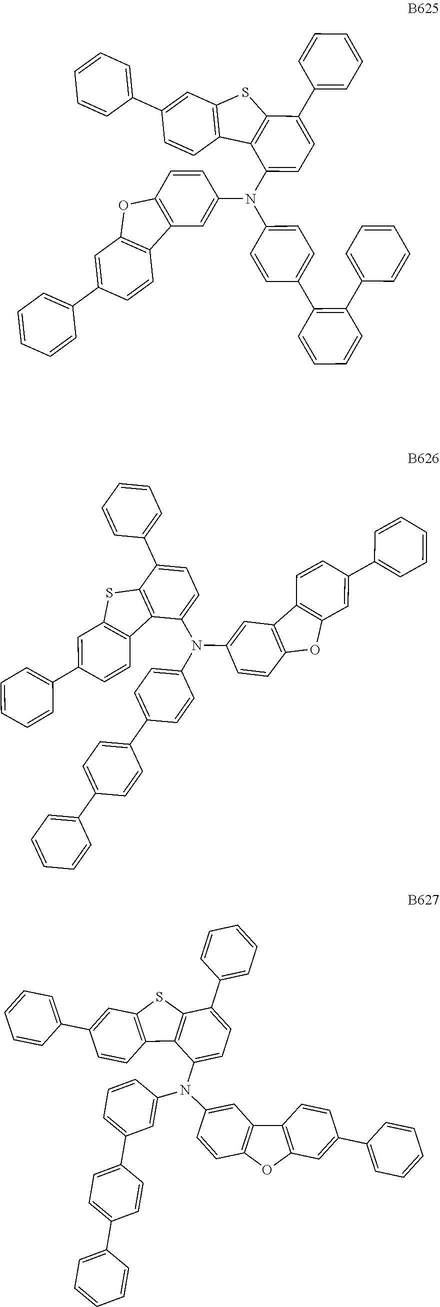 [SMILES: c1ccc(-c2ccc3c(c2)oc2ccc(N(c4ccc(-c5ccccc5-c5ccccc5)cc4)c4ccc(-c5ccccc5)c5sc6cc(-c7ccccc7)ccc6c45)cc23)cc1 ; c1ccc(-c2ccc(-c3ccc(N(c4ccc5oc6cc(-c7ccccc7)ccc6c5c4)c4ccc(-c5ccccc5)c5sc6cc(-c7ccccc7)ccc6c45)cc3)cc2)cc1 ; c1ccc(-c2ccc(-c3cccc(N(c4ccc5oc6cc(-c7ccccc7)ccc6c5c4)c4ccc(-c5ccccc5)c5sc6cc(-c7ccccc7)ccc6c45)c3)cc2)cc1]

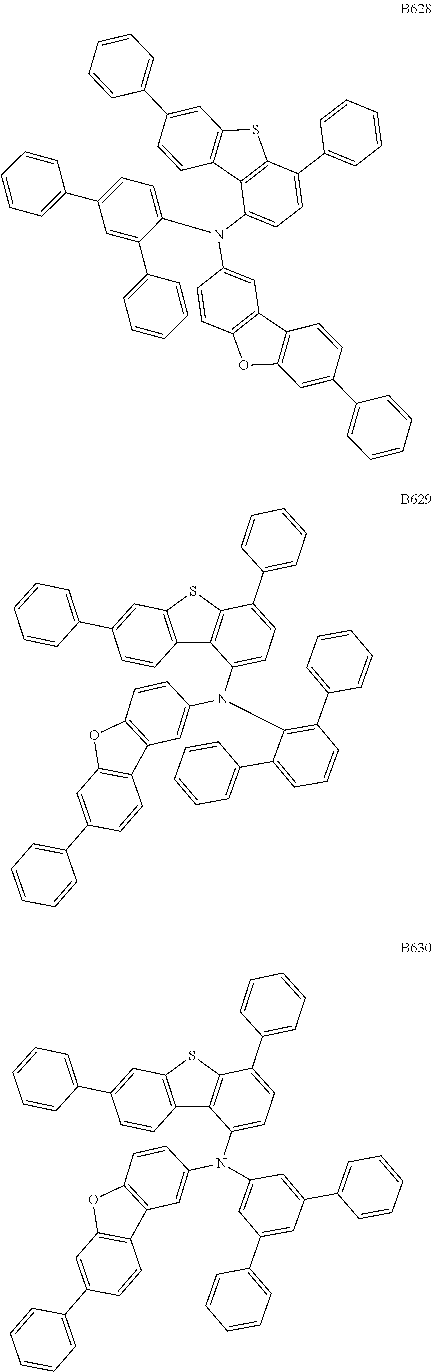 [SMILES: c1ccc(-c2ccc(N(c3ccc4oc5cc(-c6ccccc6)ccc5c4c3)c3ccc(-c4ccccc4)c4sc5cc(-c6ccccc6)ccc5c34)c(-c3ccccc3)c2)cc1 ; c1ccc(-c2ccc3c(c2)oc2ccc(N(c4c(-c5ccccc5)cccc4-c4ccccc4)c4ccc(-c5ccccc5)c5sc6cc(-c7ccccc7)ccc6c45)cc23)cc1 ; c1ccc(-c2cc(-c3ccccc3)cc(N(c3ccc4oc5cc(-c6ccccc6)ccc5c4c3)c3ccc(-c4ccccc4)c4sc5cc(-c6ccccc6)ccc5c34)c2)cc1]

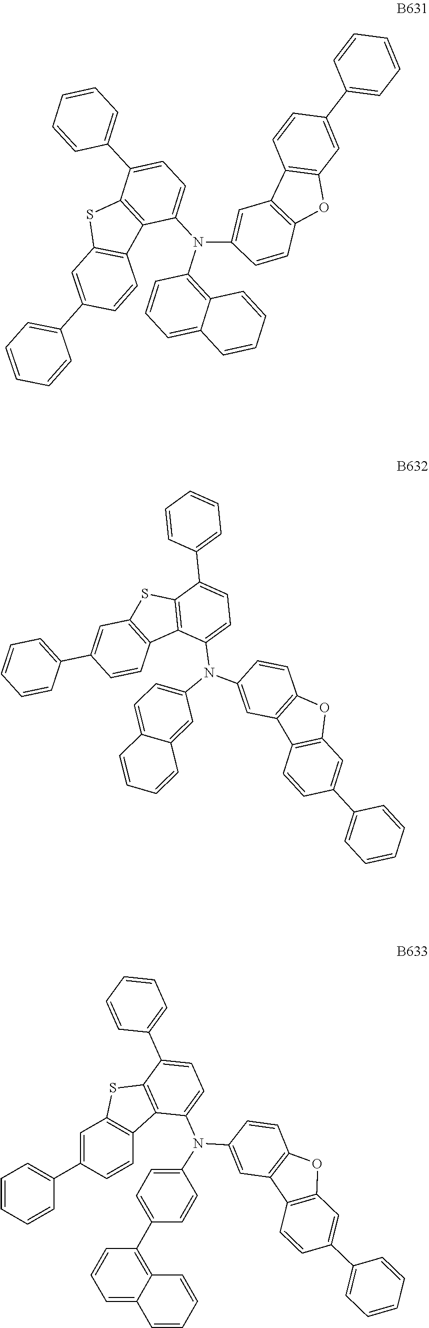 [SMILES: c1ccc(-c2ccc3c(c2)oc2ccc(N(c4cccc5ccccc45)c4ccc(-c5ccccc5)c5sc6cc(-c7ccccc7)ccc6c45)cc23)cc1 ; c1ccc(-c2ccc3c(c2)oc2ccc(N(c4ccc5ccccc5c4)c4ccc(-c5ccccc5)c5sc6cc(-c7ccccc7)ccc6c45)cc23)cc1 ; c1ccc(-c2ccc3c(c2)oc2ccc(N(c4ccc(-c5cccc6ccccc56)cc4)c4ccc(-c5ccccc5)c5sc6cc(-c7ccccc7)ccc6c45)cc23)cc1]

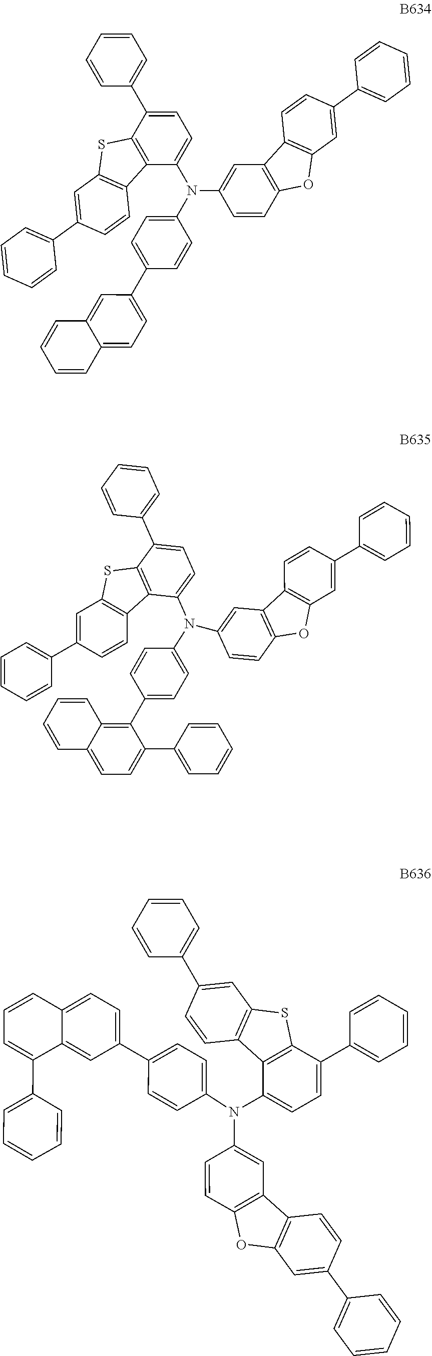 [SMILES: c1ccc(-c2ccc3c(c2)oc2ccc(N(c4ccc(-c5ccc6ccccc6c5)cc4)c4ccc(-c5ccccc5)c5sc6cc(-c7ccccc7)ccc6c45)cc23)cc1 ; c1ccc(-c2ccc3c(c2)oc2ccc(N(c4ccc(-c5c(-c6ccccc6)ccc6ccccc56)cc4)c4ccc(-c5ccccc5)c5sc6cc(-c7ccccc7)ccc6c45)cc23)cc1 ; c1ccc(-c2ccc3c(c2)oc2ccc(N(c4ccc(-c5ccc6cccc(-c7ccccc7)c6c5)cc4)c4ccc(-c5ccccc5)c5sc6cc(-c7ccccc7)ccc6c45)cc23)cc1]

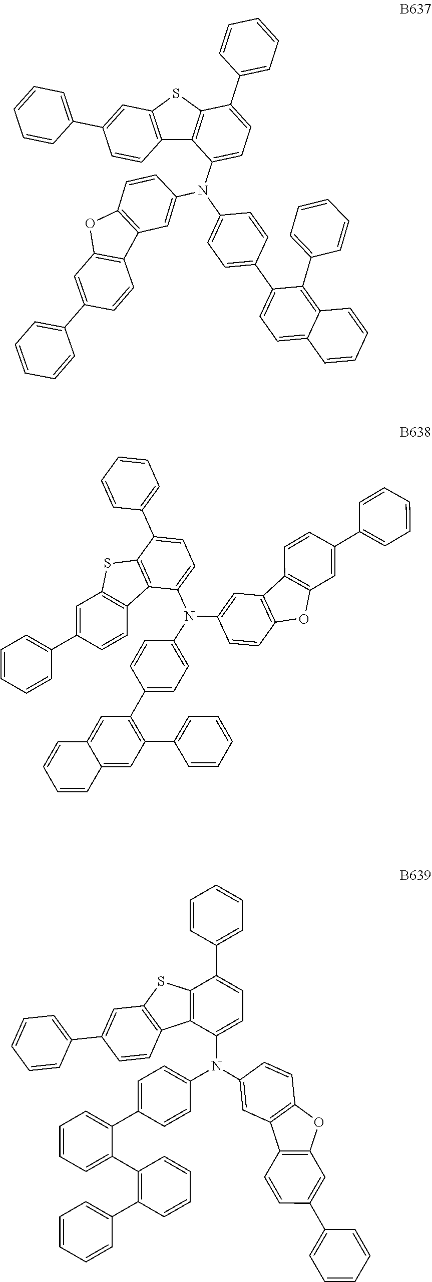 [SMILES: c1ccc(-c2ccc3c(c2)oc2ccc(N(c4ccc(-c5ccc6ccccc6c5-c5ccccc5)cc4)c4ccc(-c5ccccc5)c5sc6cc(-c7ccccc7)ccc6c45)cc23)cc1 ; c1ccc(-c2ccc3c(c2)oc2ccc(N(c4ccc(-c5cc6ccccc6cc5-c5ccccc5)cc4)c4ccc(-c5ccccc5)c5sc6cc(-c7ccccc7)ccc6c45)cc23)cc1 ; c1ccc(-c2ccc3c(c2)oc2ccc(N(c4ccc(-c5ccccc5-c5ccccc5-c5ccccc5)cc4)c4ccc(-c5ccccc5)c5sc6cc(-c7ccccc7)ccc6c45)cc23)cc1]

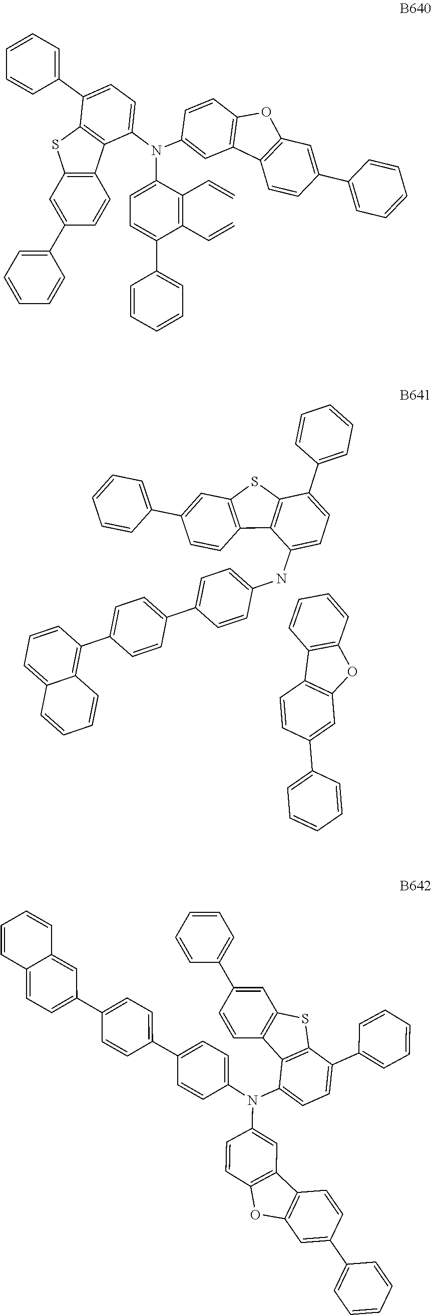

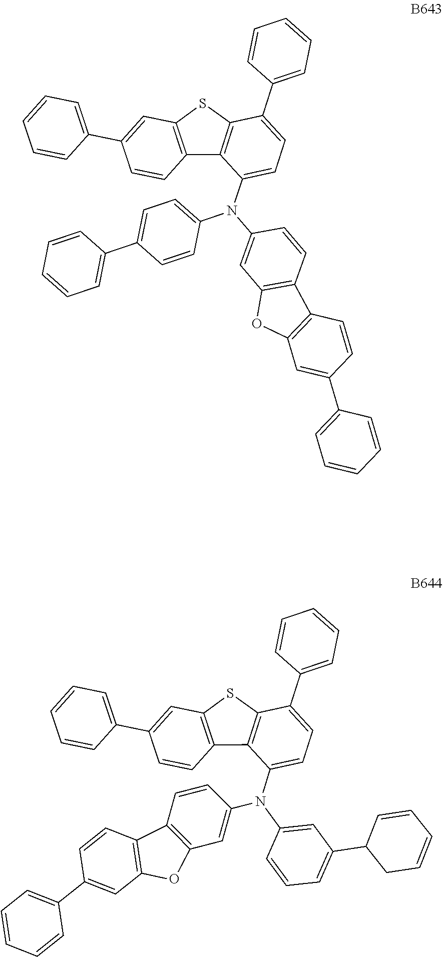 [SMILES: c1ccc(-c2ccc(N(c3ccc4c(c3)oc3cc(-c5ccccc5)ccc34)c3ccc(-c4ccccc4)c4sc5cc(-c6ccccc6)ccc5c34)cc2)cc1 ; C1=CCC(c2cccc(N(c3ccc4c(c3)oc3cc(-c5ccccc5)ccc34)c3ccc(-c4ccccc4)c4sc5cc(-c6ccccc6)ccc5c34)c2)C=C1]

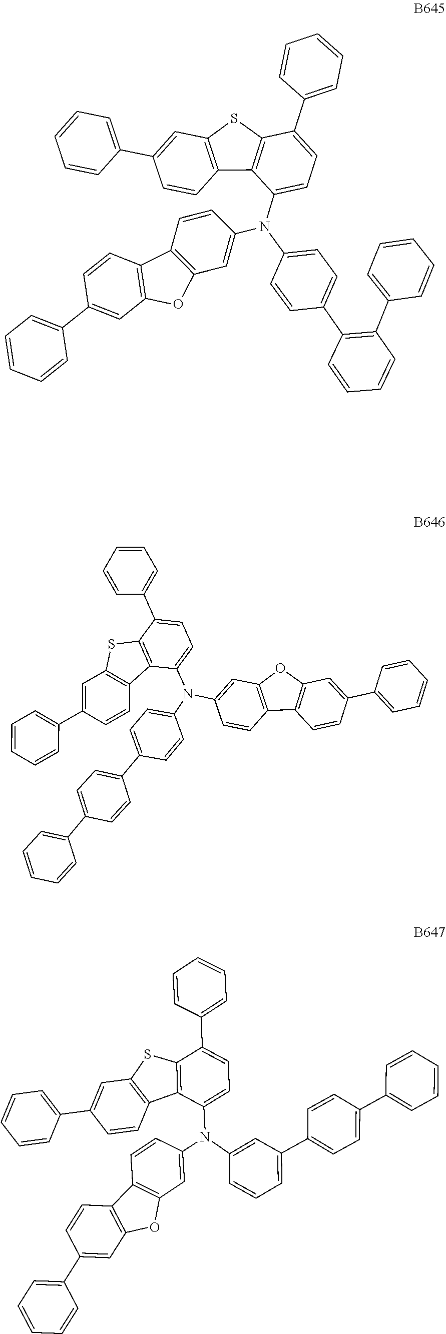 [SMILES: c1ccc(-c2ccc3c(c2)oc2cc(N(c4ccc(-c5ccccc5-c5ccccc5)cc4)c4ccc(-c5ccccc5)c5sc6cc(-c7ccccc7)ccc6c45)ccc23)cc1 ; c1ccc(-c2ccc(-c3ccc(N(c4ccc5c(c4)oc4cc(-c6ccccc6)ccc45)c4ccc(-c5ccccc5)c5sc6cc(-c7ccccc7)ccc6c45)cc3)cc2)cc1 ; c1ccc(-c2ccc(-c3cccc(N(c4ccc5c(c4)oc4cc(-c6ccccc6)ccc45)c4ccc(-c5ccccc5)c5sc6cc(-c7ccccc7)ccc6c45)c3)cc2)cc1]

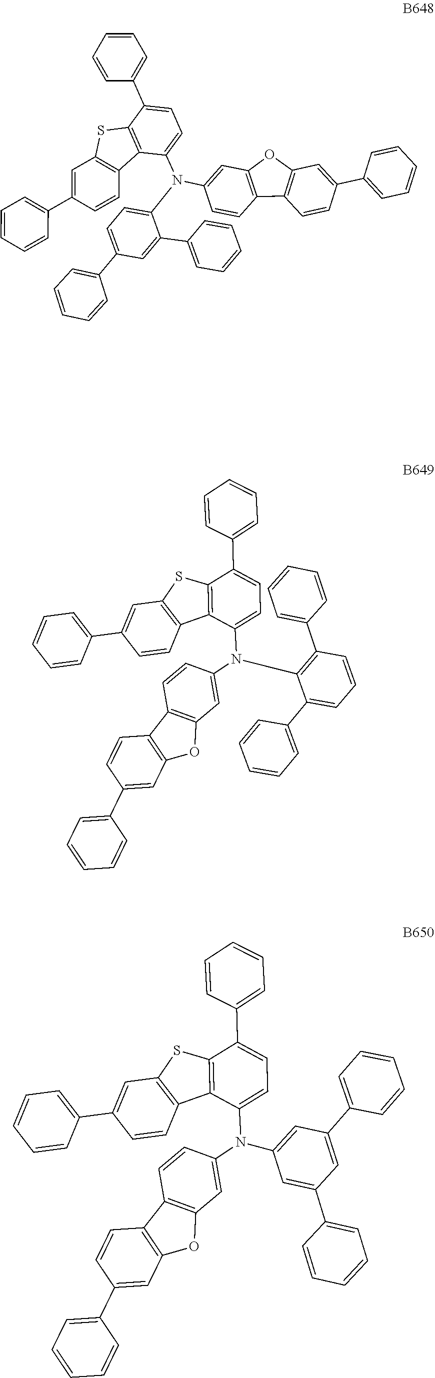 [SMILES: c1ccc(-c2ccc(N(c3ccc4c(c3)oc3cc(-c5ccccc5)ccc34)c3ccc(-c4ccccc4)c4sc5cc(-c6ccccc6)ccc5c34)c(-c3ccccc3)c2)cc1 ; c1ccc(-c2ccc3c(c2)oc2cc(N(c4c(-c5ccccc5)cccc4-c4ccccc4)c4ccc(-c5ccccc5)c5sc6cc(-c7ccccc7)ccc6c45)ccc23)cc1 ; c1ccc(-c2cc(-c3ccccc3)cc(N(c3ccc4c(c3)oc3cc(-c5ccccc5)ccc34)c3ccc(-c4ccccc4)c4sc5cc(-c6ccccc6)ccc5c34)c2)cc1]

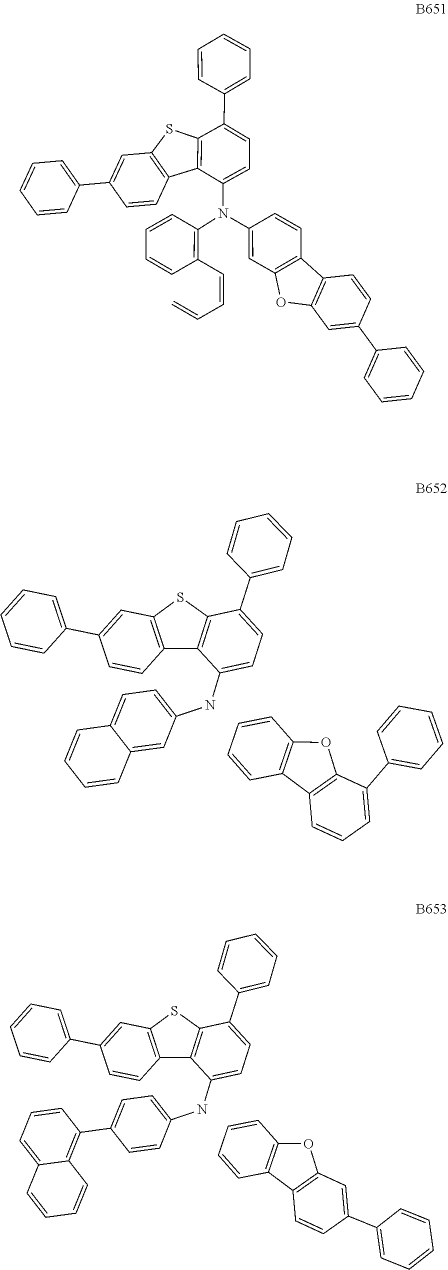

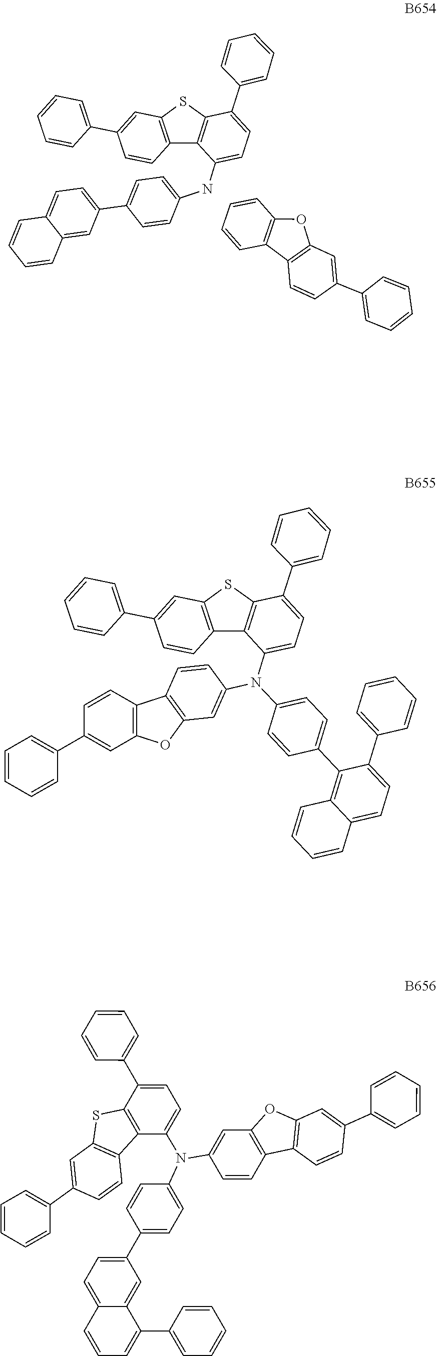

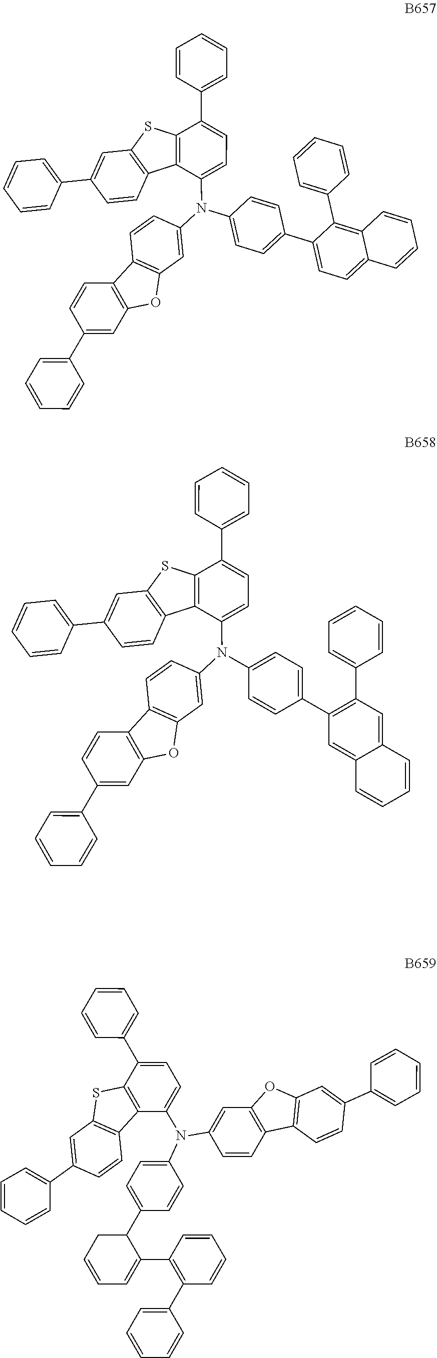 [SMILES: c1ccc(-c2ccc3c(c2)oc2cc(N(c4ccc(-c5ccc6ccccc6c5-c5ccccc5)cc4)c4ccc(-c5ccccc5)c5sc6cc(-c7ccccc7)ccc6c45)ccc23)cc1 ; c1ccc(-c2ccc3c(c2)oc2cc(N(c4ccc(-c5cc6ccccc6cc5-c5ccccc5)cc4)c4ccc(-c5ccccc5)c5sc6cc(-c7ccccc7)ccc6c45)ccc23)cc1 ; C1=CCC(c2ccc(N(c3ccc4c(c3)oc3cc(-c5ccccc5)ccc34)c3ccc(-c4ccccc4)c4sc5cc(-c6ccccc6)ccc5c34)cc2)C(c2ccccc2-c2ccccc2)=C1]

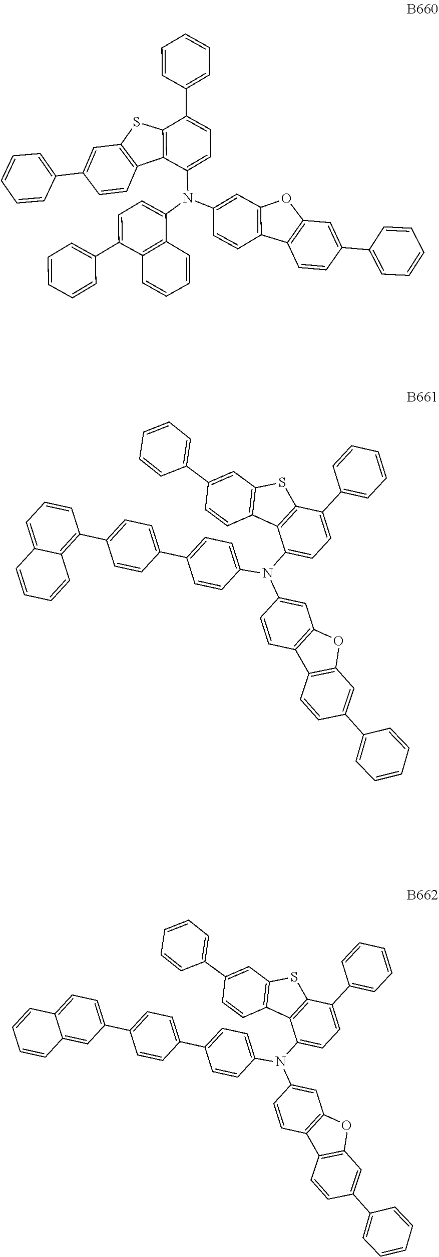 [SMILES: c1ccc(-c2ccc3c(c2)oc2cc(N(c4ccc(-c5ccccc5)c5ccccc45)c4ccc(-c5ccccc5)c5sc6cc(-c7ccccc7)ccc6c45)ccc23)cc1 ; c1ccc(-c2ccc3c(c2)oc2cc(N(c4ccc(-c5ccc(-c6cccc7ccccc67)cc5)cc4)c4ccc(-c5ccccc5)c5sc6cc(-c7ccccc7)ccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5sc6cc(-c7ccccc7)ccc6c45)ccc23)cc1]

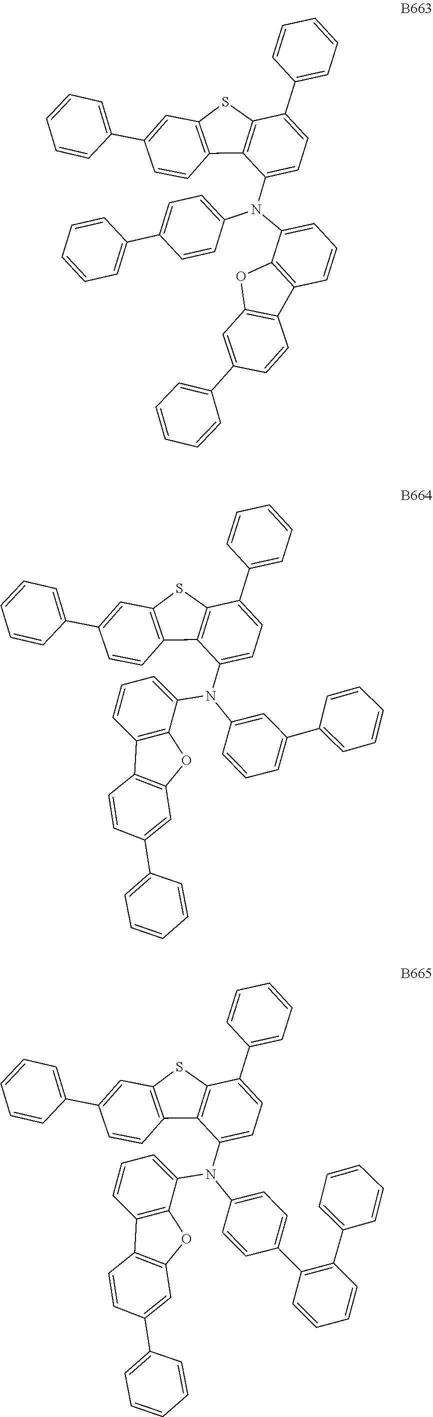 [SMILES: c1ccc(-c2ccc(N(c3cccc4c3oc3cc(-c5ccccc5)ccc34)c3ccc(-c4ccccc4)c4sc5cc(-c6ccccc6)ccc5c34)cc2)cc1 ; c1ccc(-c2cccc(N(c3cccc4c3oc3cc(-c5ccccc5)ccc34)c3ccc(-c4ccccc4)c4sc5cc(-c6ccccc6)ccc5c34)c2)cc1 ; c1ccc(-c2ccc3c(c2)oc2c(N(c4ccc(-c5ccccc5-c5ccccc5)cc4)c4ccc(-c5ccccc5)c5sc6cc(-c7ccccc7)ccc6c45)cccc23)cc1]

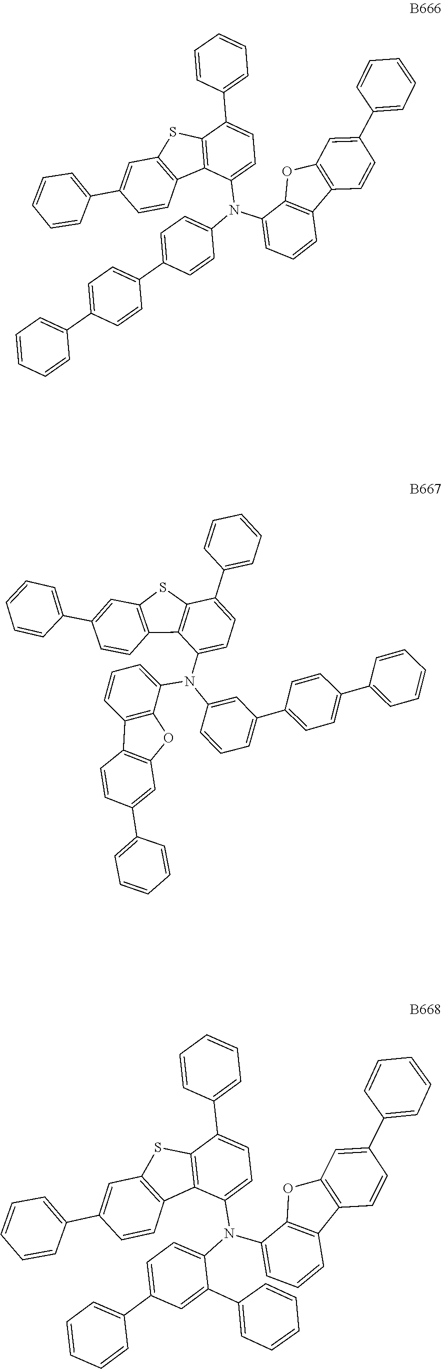 [SMILES: c1ccc(-c2ccc(-c3ccc(N(c4cccc5c4oc4cc(-c6ccccc6)ccc45)c4ccc(-c5ccccc5)c5sc6cc(-c7ccccc7)ccc6c45)cc3)cc2)cc1 ; c1ccc(-c2ccc(-c3cccc(N(c4cccc5c4oc4cc(-c6ccccc6)ccc45)c4ccc(-c5ccccc5)c5sc6cc(-c7ccccc7)ccc6c45)c3)cc2)cc1 ; c1ccc(-c2ccc(N(c3cccc4c3oc3cc(-c5ccccc5)ccc34)c3ccc(-c4ccccc4)c4sc5cc(-c6ccccc6)ccc5c34)c(-c3ccccc3)c2)cc1]

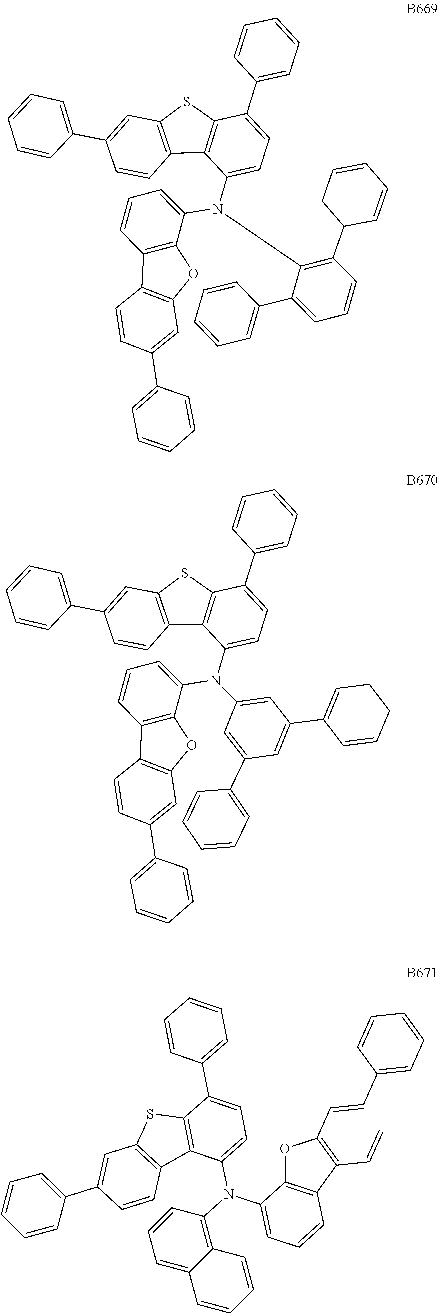 [SMILES: C1=CCC(c2cccc(-c3ccccc3)c2N(c2cccc3c2oc2cc(-c4ccccc4)ccc23)c2ccc(-c3ccccc3)c3sc4cc(-c5ccccc5)ccc4c23)C=C1 ; C1=CC(c2cc(-c3ccccc3)cc(N(c3cccc4c3oc3cc(-c5ccccc5)ccc34)c3ccc(-c4ccccc4)c4sc5cc(-c6ccccc6)ccc5c34)c2)=CCC1 ; C=Cc1c(/C=C/c2ccccc2)oc2c(N(c3cccc4ccccc34)c3ccc(-c4ccccc4)c4sc5cc(-c6ccccc6)ccc5c34)cccc12]

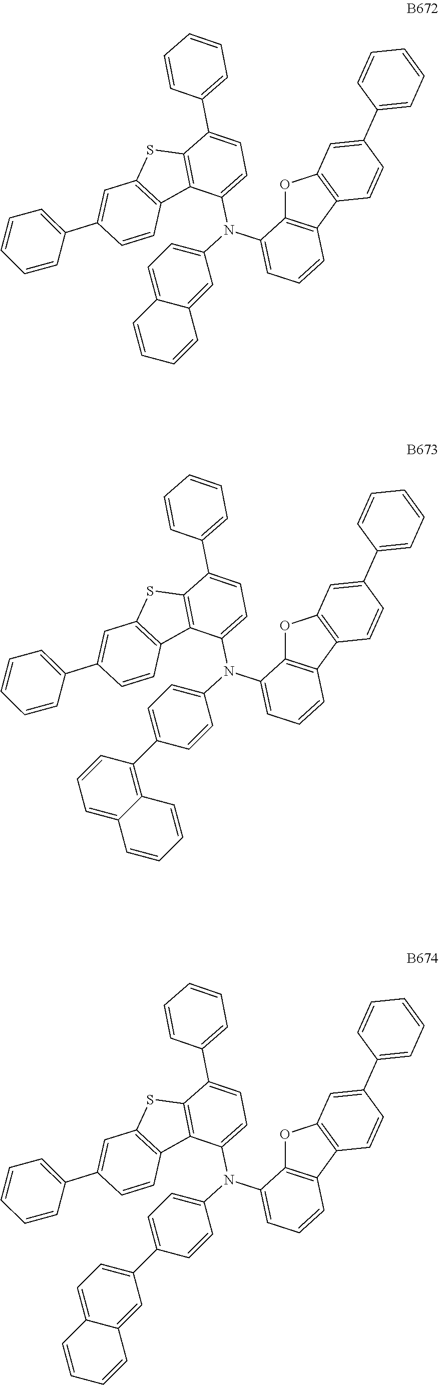 [SMILES: c1ccc(-c2ccc3c(c2)oc2c(N(c4ccc5ccccc5c4)c4ccc(-c5ccccc5)c5sc6cc(-c7ccccc7)ccc6c45)cccc23)cc1 ; c1ccc(-c2ccc3c(c2)oc2c(N(c4ccc(-c5cccc6ccccc56)cc4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6ccccc6c5)cc4)c4ccc(-c5ccccc5)c5sc6cc(-c7ccccc7)ccc6c45)cccc23)cc1]

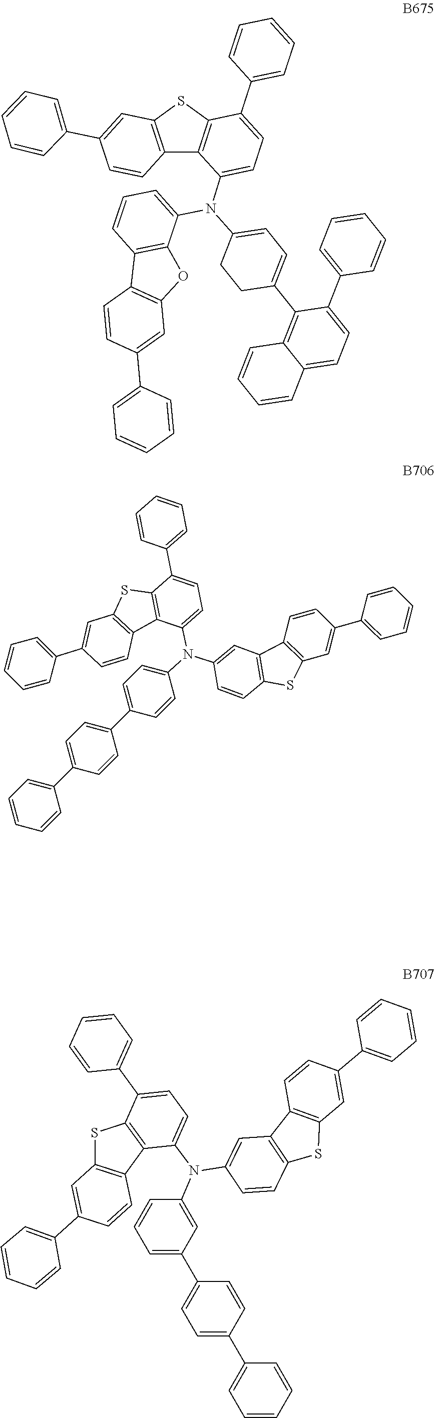 [SMILES: C1=C(c2c(-c3ccccc3)ccc3ccccc23)CCC(N(c2cccc3c2oc2cc(-c4ccccc4)ccc23)c2ccc(-c3ccccc3)c3sc4cc(-c5ccccc5)ccc4c23)=C1 ; c1ccc(-c2ccc(-c3ccc(N(c4ccc5sc6cc(-c7ccccc7)ccc6c5c4)c4ccc(-c5ccccc5)c5sc6cc(-c7ccccc7)ccc6c45)cc3)cc2)cc1 ; c1ccc(-c2ccc(-c3cccc(N(c4ccc5sc6cc(-c7ccccc7)ccc6c5c4)c4ccc(-c5ccccc5)c5sc6cc(-c7ccccc7)ccc6c45)c3)cc2)cc1]

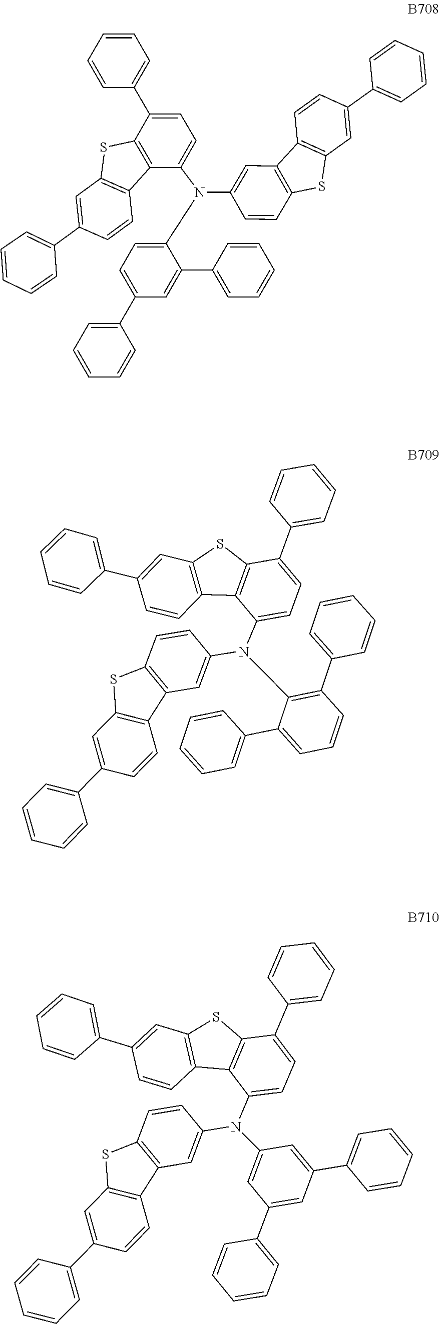 [SMILES: c1ccc(-c2ccc(N(c3ccc4sc5cc(-c6ccccc6)ccc5c4c3)c3ccc(-c4ccccc4)c4sc5cc(-c6ccccc6)ccc5c34)c(-c3ccccc3)c2)cc1 ; c1ccc(-c2ccc3c(c2)sc2ccc(N(c4c(-c5ccccc5)cccc4-c4ccccc4)c4ccc(-c5ccccc5)c5sc6cc(-c7ccccc7)ccc6c45)cc23)cc1 ; c1ccc(-c2cc(-c3ccccc3)cc(N(c3ccc4sc5cc(-c6ccccc6)ccc5c4c3)c3ccc(-c4ccccc4)c4sc5cc(-c6ccccc6)ccc5c34)c2)cc1]

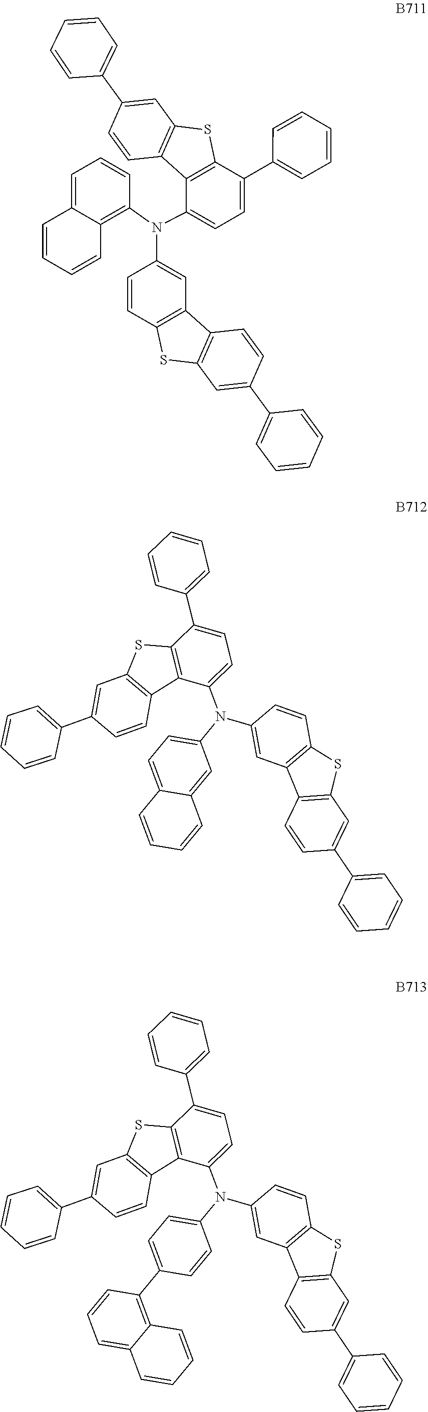 [SMILES: c1ccc(-c2ccc3c(c2)sc2ccc(N(c4cccc5ccccc45)c4ccc(-c5ccccc5)c5sc6cc(-c7ccccc7)ccc6c45)cc23)cc1 ; c1ccc(-c2ccc3c(c2)sc2ccc(N(c4ccc5ccccc5c4)c4ccc(-c5ccccc5)c5sc6cc(-c7ccccc7)ccc6c45)cc23)cc1 ; c1ccc(-c2ccc3c(c2)sc2ccc(N(c4ccc(-c5cccc6ccccc56)cc4)c4ccc(-c5ccccc5)c5sc6cc(-c7ccccc7)ccc6c45)cc23)cc1]

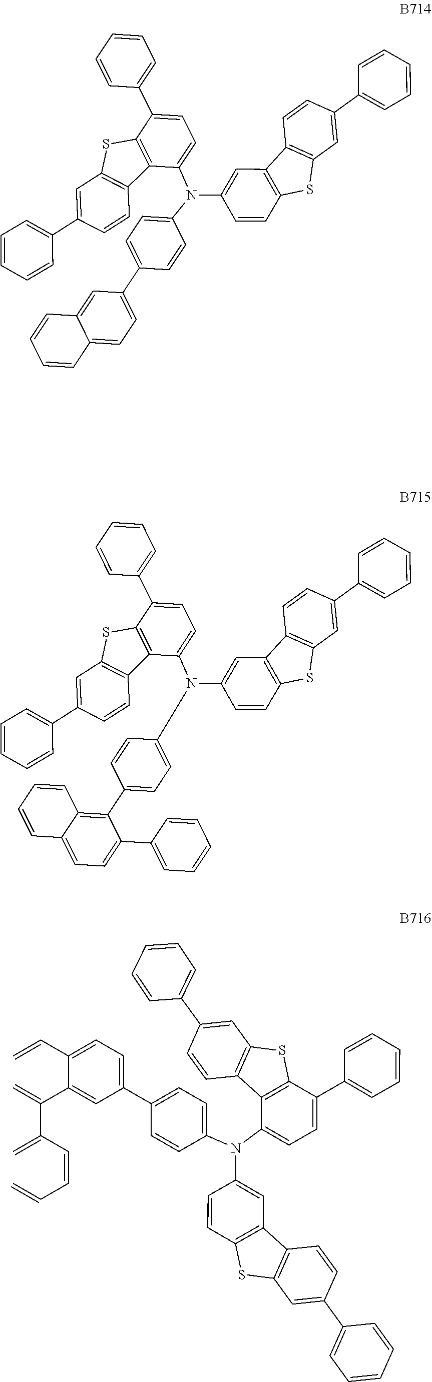 [SMILES: c1ccc(-c2ccc3c(c2)sc2ccc(N(c4ccc(-c5ccc6ccccc6c5)cc4)c4ccc(-c5ccccc5)c5sc6cc(-c7ccccc7)ccc6c45)cc23)cc1 ; c1ccc(-c2ccc3c(c2)sc2ccc(N(c4ccc(-c5c(-c6ccccc6)ccc6ccccc56)cc4)c4ccc(-c5ccccc5)c5sc6cc(-c7ccccc7)ccc6c45)cc23)cc1 ; C=C/C=C\C(=C)C(=C)c1cc(-c2ccc(N(c3ccc4sc5cc(-c6ccccc6)ccc5c4c3)c3ccc(-c4ccccc4)c4sc5cc(-c6ccccc6)ccc5c34)cc2)ccc1C=C]

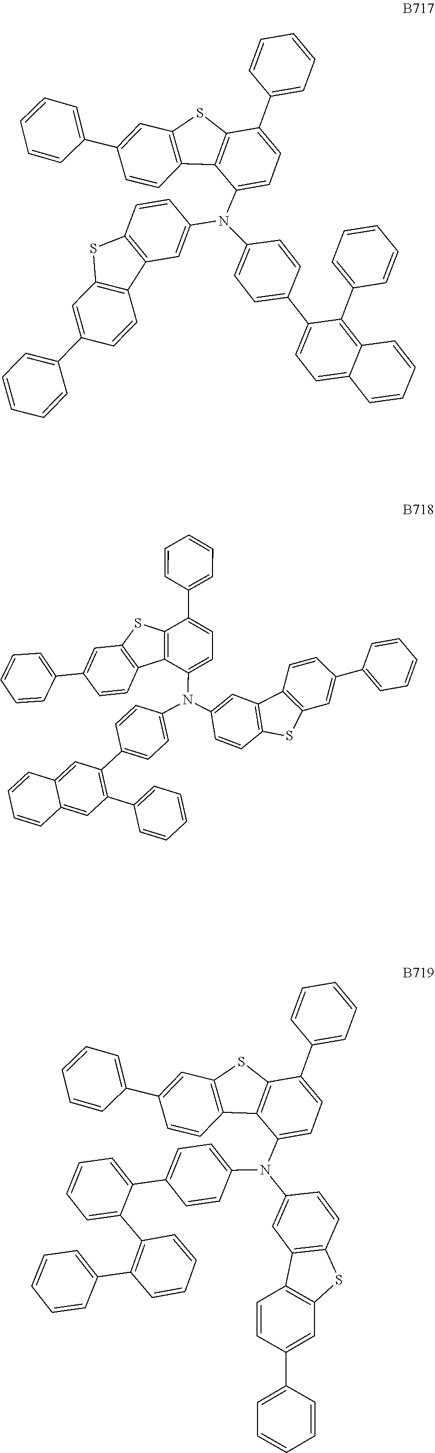

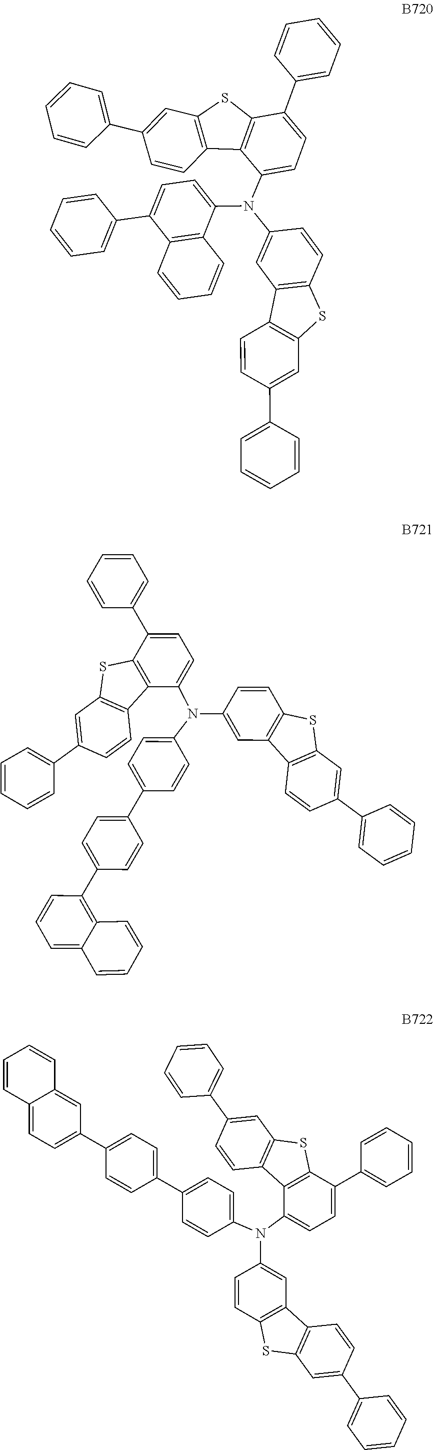

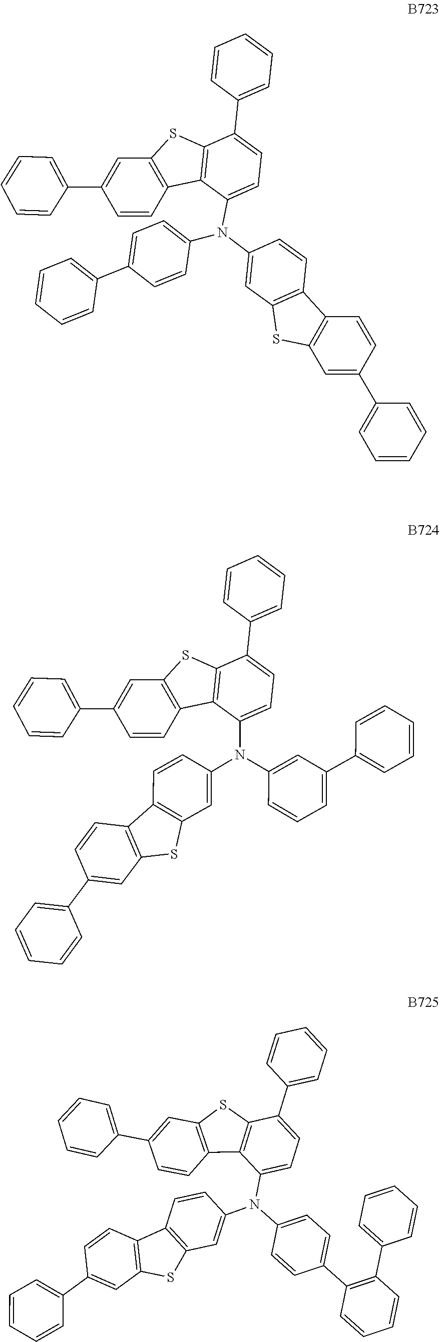

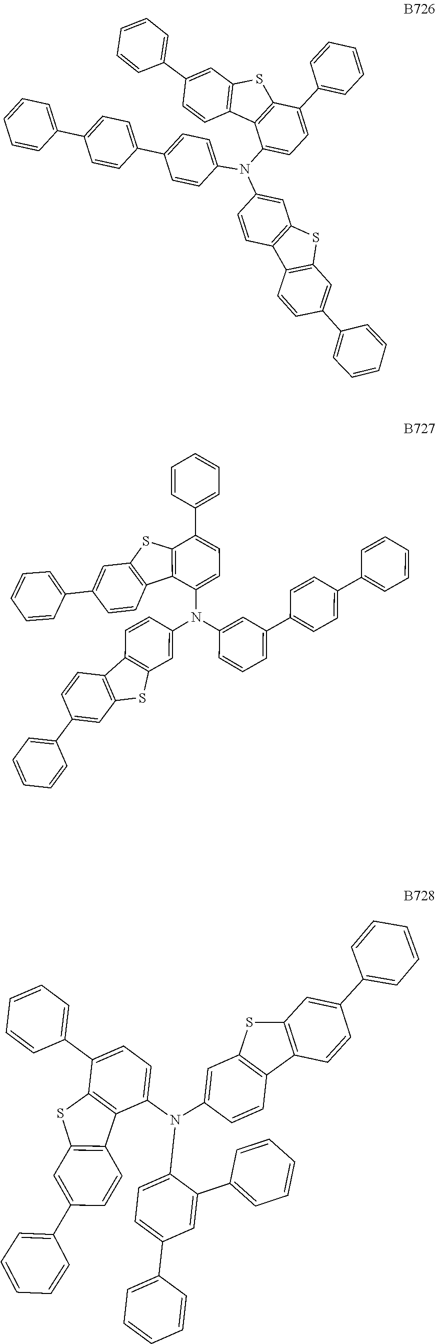

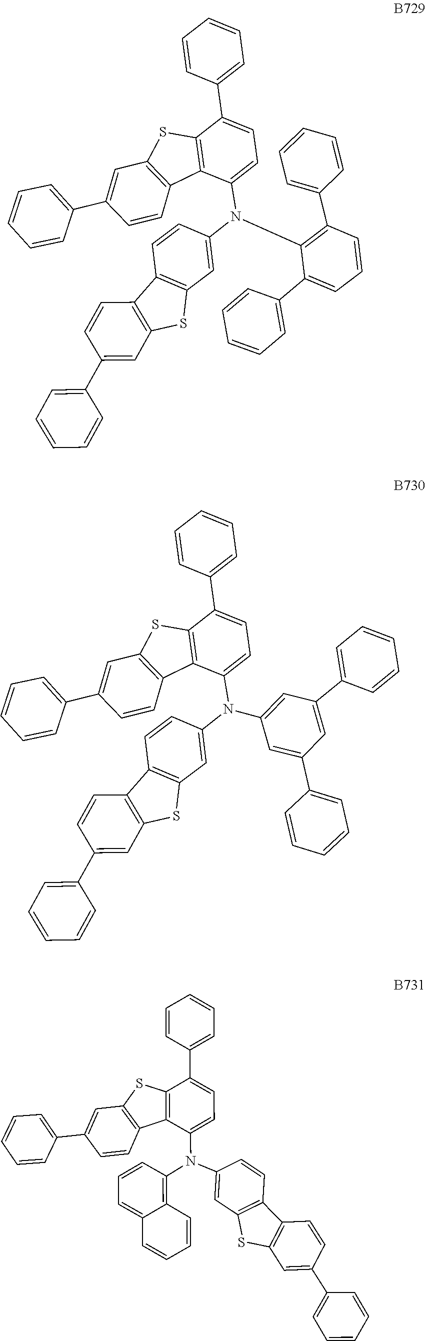 [SMILES: c1ccc(-c2ccc3c(c2)sc2cc(N(c4c(-c5ccccc5)cccc4-c4ccccc4)c4ccc(-c5ccccc5)c5sc6cc(-c7ccccc7)ccc6c45)ccc23)cc1 ; c1ccc(-c2cc(-c3ccccc3)cc(N(c3ccc4c(c3)sc3cc(-c5ccccc5)ccc34)c3ccc(-c4ccccc4)c4sc5cc(-c6ccccc6)ccc5c34)c2)cc1 ; c1ccc(-c2ccc3c(c2)sc2cc(N(c4cccc5ccccc45)c4ccc(-c5ccccc5)c5sc6cc(-c7ccccc7)ccc6c45)ccc23)cc1]

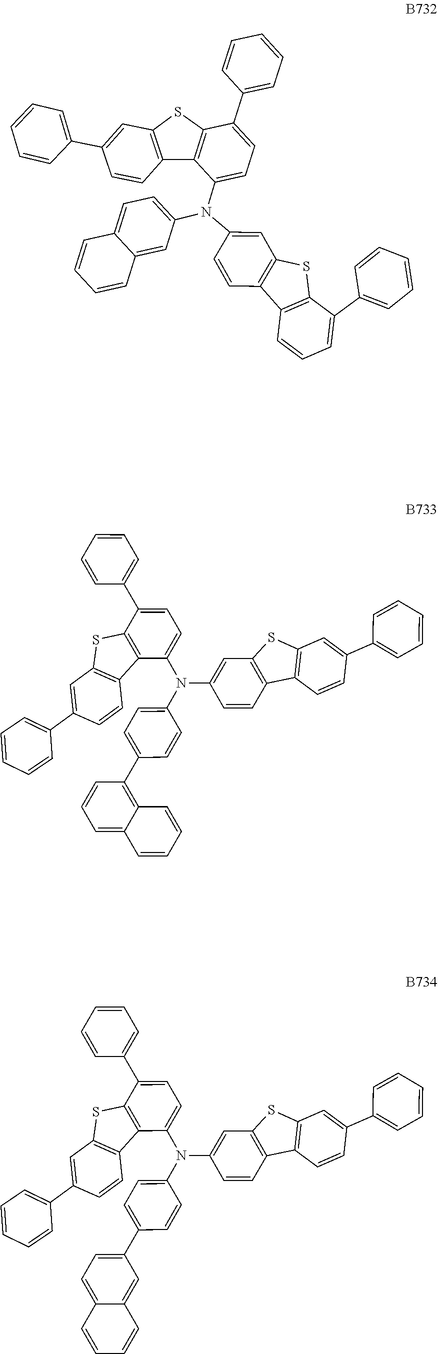 [SMILES: c1ccc(-c2ccc3c(c2)sc2c(-c4ccccc4)ccc(N(c4ccc5ccccc5c4)c4ccc5c(c4)sc4c(-c6ccccc6)cccc45)c23)cc1 ; c1ccc(-c2ccc3c(c2)sc2cc(N(c4ccc(-c5cccc6ccccc56)cc4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc6ccccc6c5)cc4)c4ccc(-c5ccccc5)c5sc6cc(-c7ccccc7)ccc6c45)ccc23)cc1]

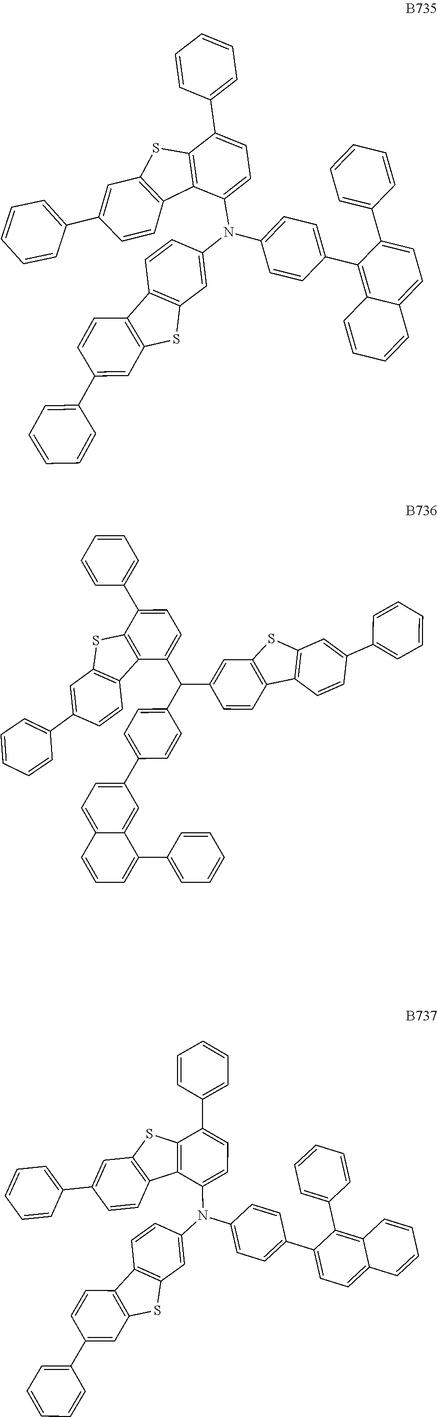 [SMILES: c1ccc(-c2ccc3c(c2)sc2cc(N(c4ccc(-c5c(-c6ccccc6)ccc6ccccc56)cc4)c4ccc(-c5ccccc5)c5sc6cc(-c7ccccc7)ccc6c45)ccc23)cc1 ; c1ccc(-c2ccc3c(c2)sc2cc(C(c4ccc(-c5ccc6cccc(-c7ccccc7)c6c5)cc4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc6ccccc6c5-c5ccccc5)cc4)c4ccc(-c5ccccc5)c5sc6cc(-c7ccccc7)ccc6c45)ccc23)cc1]

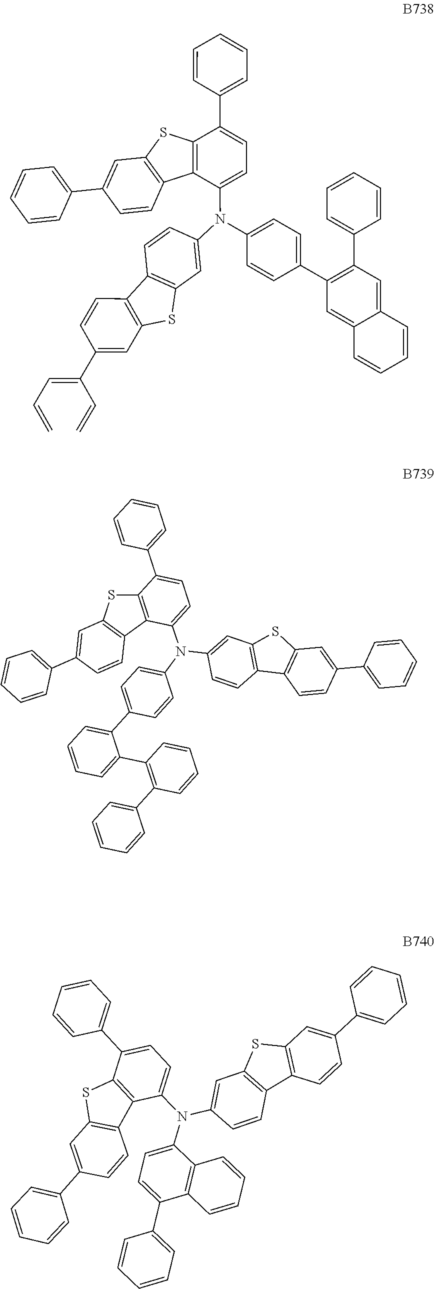 [SMILES: C=C/C=C(\C=C)c1ccc2c(c1)sc1cc(N(c3ccc(-c4cc5ccccc5cc4-c4ccccc4)cc3)c3ccc(-c4ccccc4)c4sc5cc(-c6ccccc6)ccc5c34)ccc12 ; c1ccc(-c2ccc3c(c2)sc2cc(N(c4ccc(-c5ccccc5-c5ccccc5-c5ccccc5)cc4)c4ccc(-c5ccccc5)c5sc6cc(-c7ccccc7)ccc6c45)ccc23)cc1 ; c1ccc(-c2ccc3c(c2)sc2cc(N(c4ccc(-c5ccccc5)c5ccccc45)c4ccc(-c5ccccc5)c5sc6cc(-c7ccccc7)ccc6c45)ccc23)cc1]

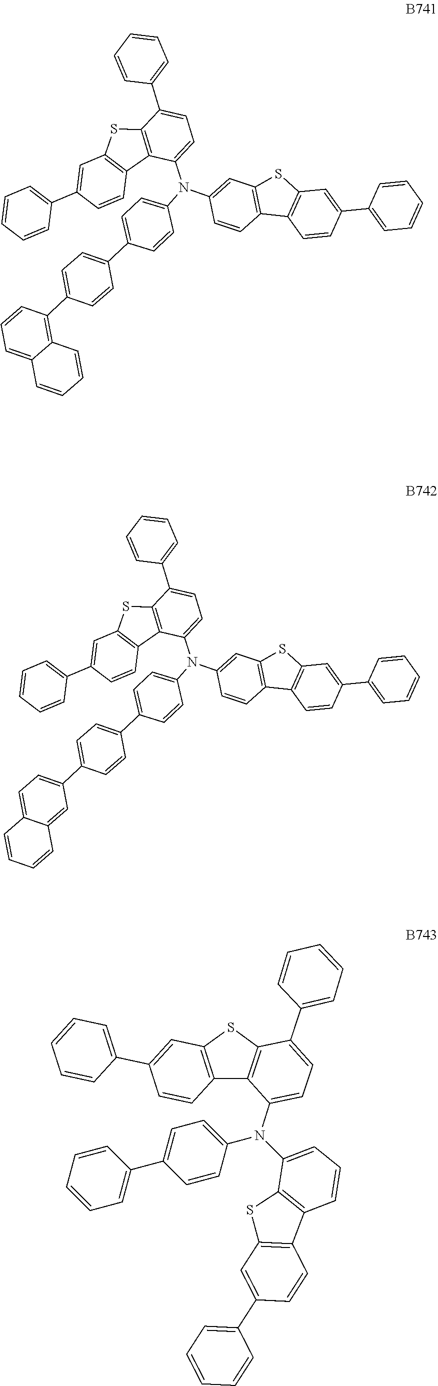 [SMILES: c1ccc(-c2ccc3c(c2)sc2cc(N(c4ccc(-c5ccc(-c6cccc7ccccc67)cc5)cc4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc(-c6ccc7ccccc7c6)cc5)cc4)c4ccc(-c5ccccc5)c5sc6cc(-c7ccccc7)ccc6c45)ccc23)cc1 ; c1ccc(-c2ccc(N(c3cccc4c3sc3cc(-c5ccccc5)ccc34)c3ccc(-c4ccccc4)c4sc5cc(-c6ccccc6)ccc5c34)cc2)cc1]

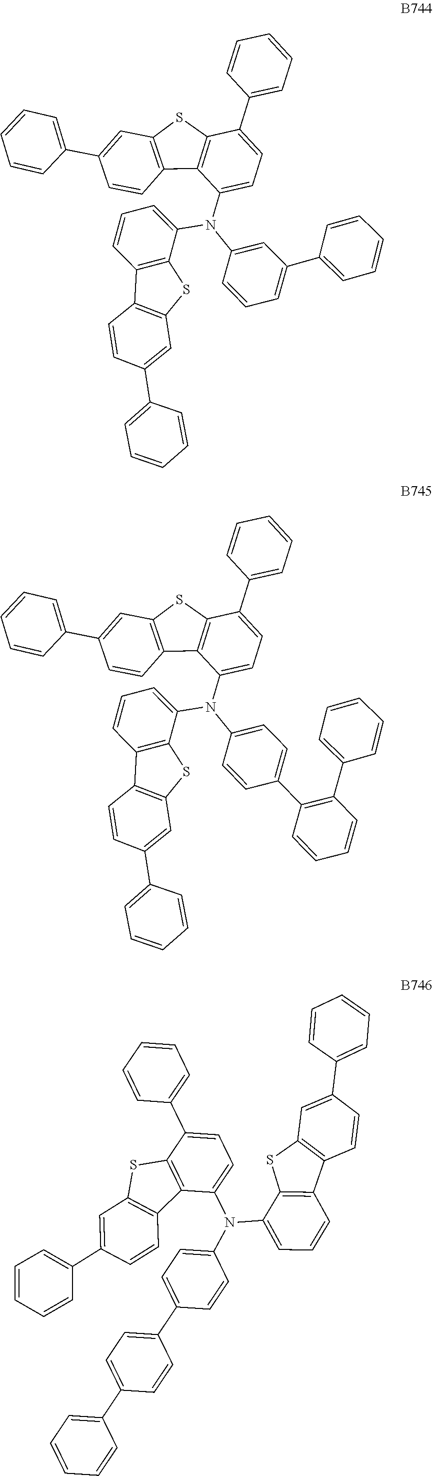 [SMILES: c1ccc(-c2cccc(N(c3cccc4c3sc3cc(-c5ccccc5)ccc34)c3ccc(-c4ccccc4)c4sc5cc(-c6ccccc6)ccc5c34)c2)cc1 ; c1ccc(-c2ccc3c(c2)sc2c(N(c4ccc(-c5ccccc5-c5ccccc5)cc4)c4ccc(-c5ccccc5)c5sc6cc(-c7ccccc7)ccc6c45)cccc23)cc1 ; c1ccc(-c2ccc(-c3ccc(N(c4cccc5c4sc4cc(-c6ccccc6)ccc45)c4ccc(-c5ccccc5)c5sc6cc(-c7ccccc7)ccc6c45)cc3)cc2)cc1]

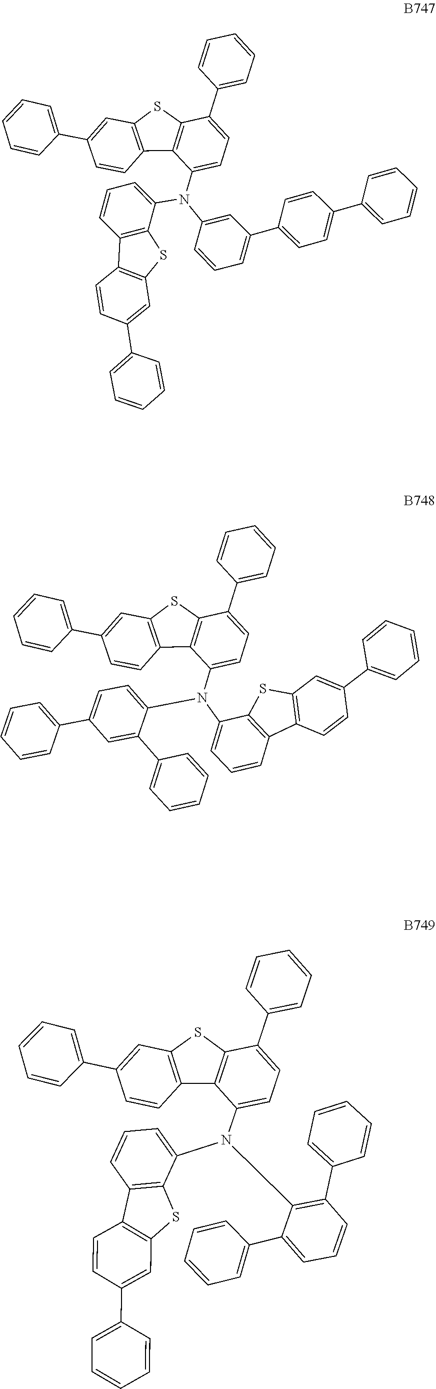 [SMILES: c1ccc(-c2ccc(-c3cccc(N(c4cccc5c4sc4cc(-c6ccccc6)ccc45)c4ccc(-c5ccccc5)c5sc6cc(-c7ccccc7)ccc6c45)c3)cc2)cc1 ; c1ccc(-c2ccc(N(c3cccc4c3sc3cc(-c5ccccc5)ccc34)c3ccc(-c4ccccc4)c4sc5cc(-c6ccccc6)ccc5c34)c(-c3ccccc3)c2)cc1 ; c1ccc(-c2ccc3c(c2)sc2c(N(c4c(-c5ccccc5)cccc4-c4ccccc4)c4ccc(-c5ccccc5)c5sc6cc(-c7ccccc7)ccc6c45)cccc23)cc1]

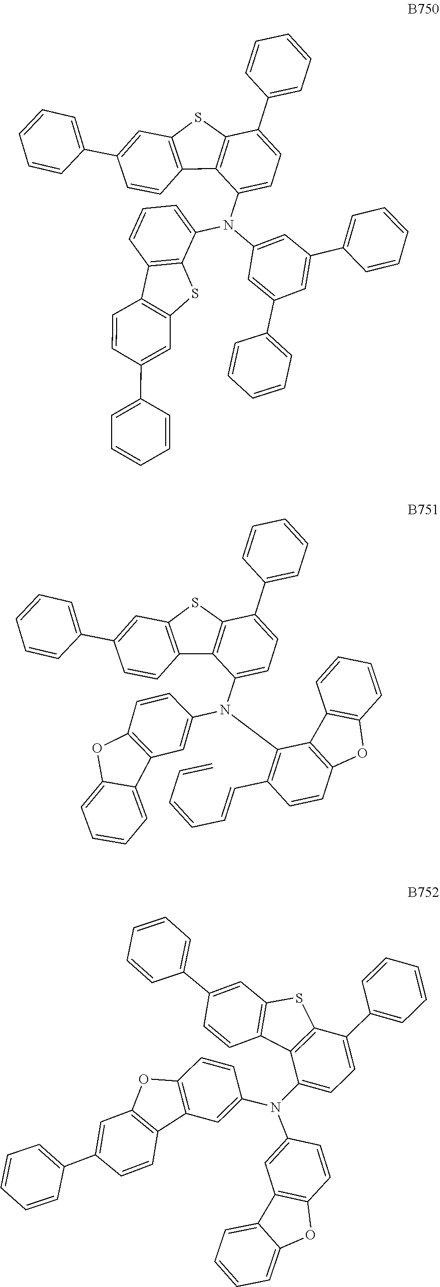 [SMILES: c1ccc(-c2cc(-c3ccccc3)cc(N(c3cccc4c3sc3cc(-c5ccccc5)ccc34)c3ccc(-c4ccccc4)c4sc5cc(-c6ccccc6)ccc5c34)c2)cc1 ; C=C/C=C\C=C\c1ccc2oc3ccccc3c2c1N(c1ccc2oc3ccccc3c2c1)c1ccc(-c2ccccc2)c2sc3cc(-c4ccccc4)ccc3c12 ; c1ccc(-c2ccc3c(c2)oc2ccc(N(c4ccc5oc6ccccc6c5c4)c4ccc(-c5ccccc5)c5sc6cc(-c7ccccc7)ccc6c45)cc23)cc1]

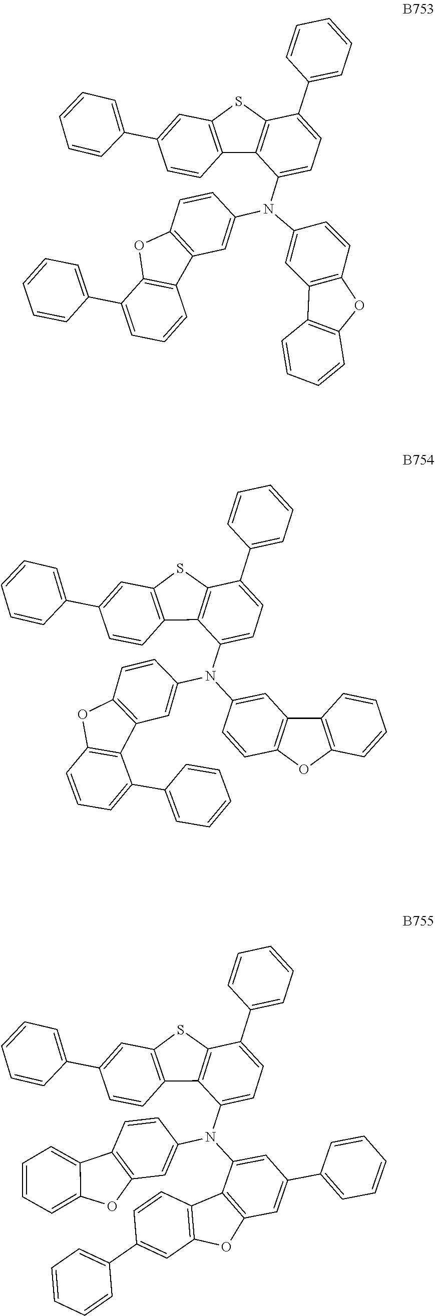 [SMILES: c1ccc(-c2ccc3c(c2)sc2c(-c4ccccc4)ccc(N(c4ccc5oc6ccccc6c5c4)c4ccc5oc6c(-c7ccccc7)cccc6c5c4)c23)cc1 ; c1ccc(-c2ccc3c(c2)sc2c(-c4ccccc4)ccc(N(c4ccc5oc6ccccc6c5c4)c4ccc5oc6cccc(-c7ccccc7)c6c5c4)c23)cc1 ; c1ccc(-c2ccc3c(c2)oc2cc(-c4ccccc4)cc(N(c4ccc5c(c4)oc4ccccc45)c4ccc(-c5ccccc5)c5sc6cc(-c7ccccc7)ccc6c45)c23)cc1]

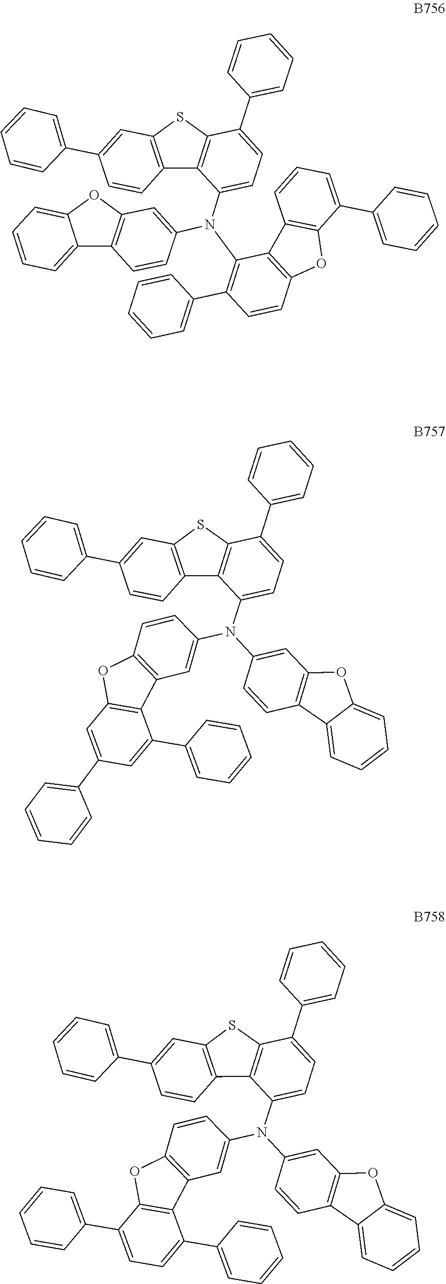 [SMILES: c1ccc(-c2ccc3c(c2)sc2c(-c4ccccc4)ccc(N(c4ccc5c(c4)oc4ccccc45)c4c(-c5ccccc5)ccc5oc6c(-c7ccccc7)cccc6c45)c23)cc1 ; c1ccc(-c2cc(-c3ccccc3)c3c(c2)oc2ccc(N(c4ccc5c(c4)oc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5c(c4)oc4ccccc45)c4ccc5oc6c(-c7ccccc7)ccc(-c7ccccc7)c6c5c4)c23)cc1]

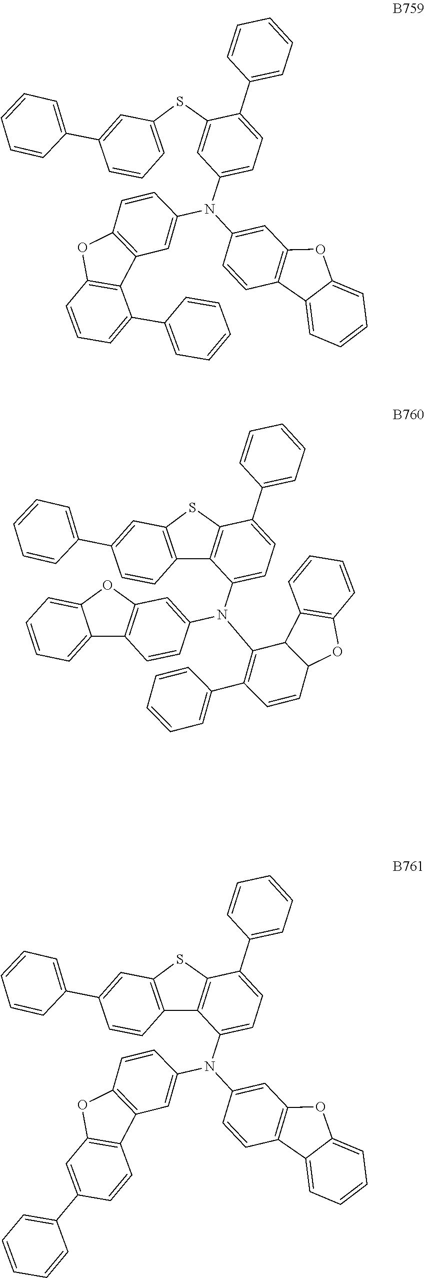 [SMILES: c1ccc(-c2cccc(Sc3cc(N(c4ccc5c(c4)oc4ccccc45)c4ccc5oc6cccc(-c7ccccc7)c6c5c4)ccc3-c3ccccc3)c2)cc1 ; C1=CC2Oc3ccccc3C2C(N(c2ccc3c(c2)oc2ccccc23)c2ccc(-c3ccccc3)c3sc4cc(-c5ccccc5)ccc4c23)=C1c1ccccc1 ; c1ccc(-c2ccc3c(c2)oc2ccc(N(c4ccc5c(c4)oc4ccccc45)c4ccc(-c5ccccc5)c5sc6cc(-c7ccccc7)ccc6c45)cc23)cc1]

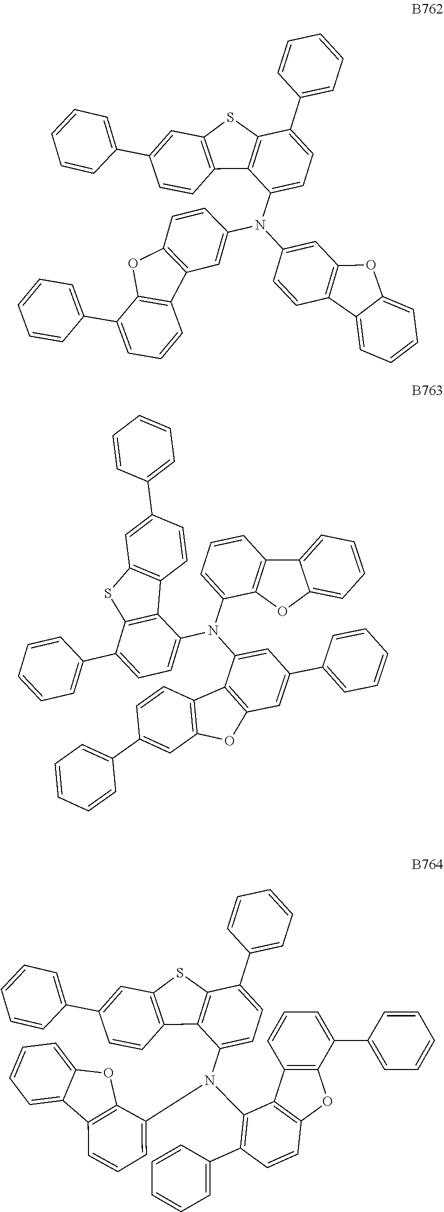 [SMILES: c1ccc(-c2ccc3c(c2)sc2c(-c4ccccc4)ccc(N(c4ccc5c(c4)oc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5c4oc4ccccc45)c4c(-c5ccccc5)ccc5oc6c(-c7ccccc7)cccc6c45)c23)cc1]

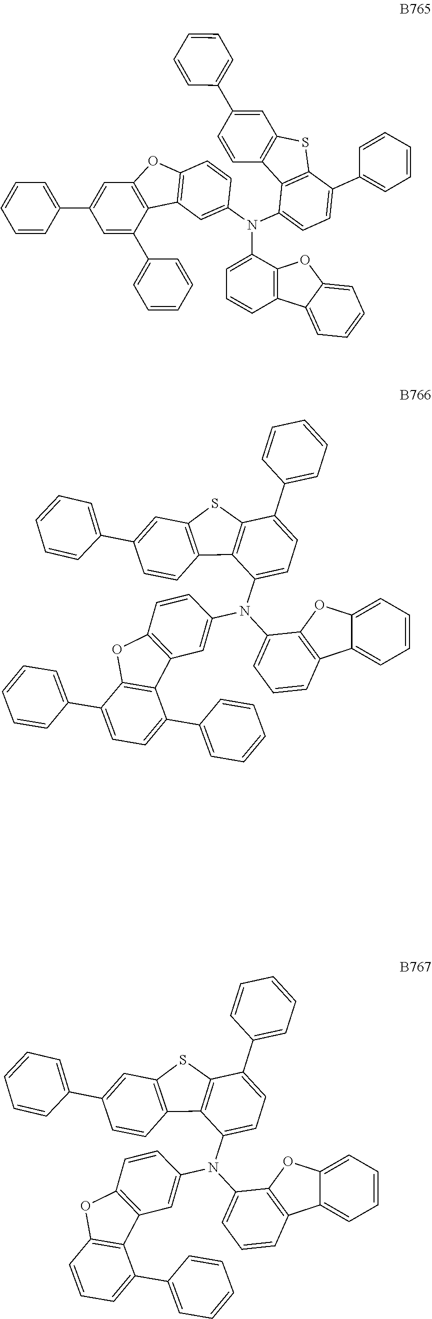 [SMILES: c1ccc(-c2cc(-c3ccccc3)c3c(c2)oc2ccc(N(c4cccc5c4oc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)ccc(-c7ccccc7)c6c5c4)c4cccc5c4oc4ccccc45)c23)cc1 ; c1ccc(-c2ccc3c(c2)sc2c(-c4ccccc4)ccc(N(c4ccc5oc6cccc(-c7ccccc7)c6c5c4)c4cccc5c4oc4ccccc45)c23)cc1]

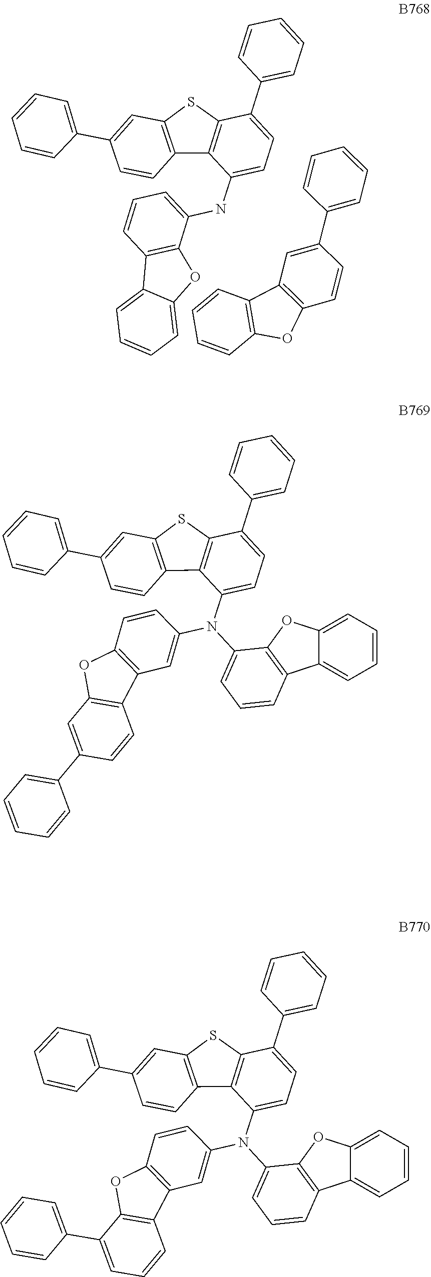

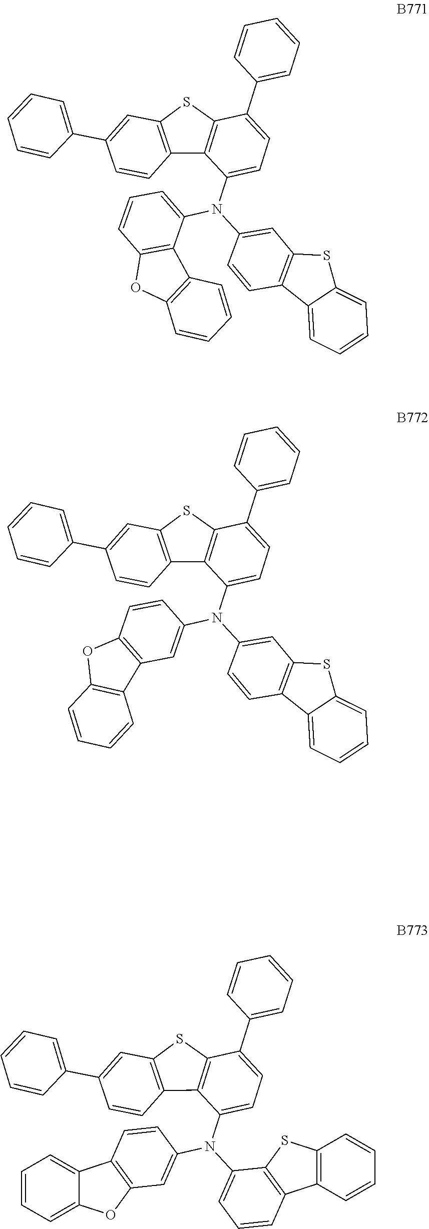 [SMILES: c1ccc(-c2ccc3c(c2)sc2c(-c4ccccc4)ccc(N(c4ccc5c(c4)sc4ccccc45)c4cccc5oc6ccccc6c45)c23)cc1 ; c1ccc(-c2ccc3c(c2)sc2c(-c4ccccc4)ccc(N(c4ccc5c(c4)sc4ccccc45)c4ccc5oc6ccccc6c5c4)c23)cc1 ; c1ccc(-c2ccc3c(c2)sc2c(-c4ccccc4)ccc(N(c4ccc5c(c4)oc4ccccc45)c4cccc5c4sc4ccccc45)c23)cc1]

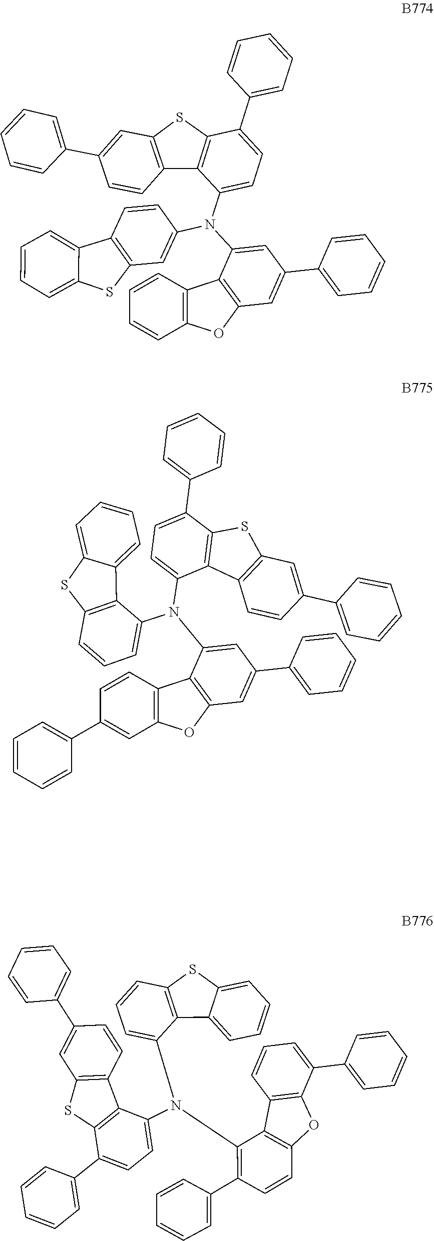

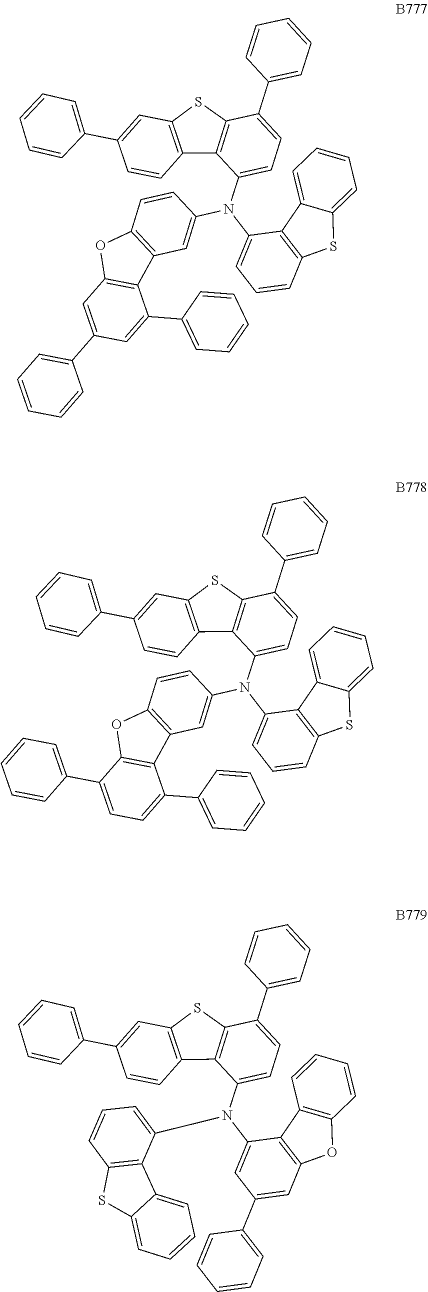 [SMILES: c1ccc(-c2cc(-c3ccccc3)c3c(c2)oc2ccc(N(c4cccc5sc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)ccc(-c7ccccc7)c6c5c4)c4cccc5sc6ccccc6c45)c23)cc1 ; c1ccc(-c2cc(N(c3cccc4sc5ccccc5c34)c3ccc(-c4ccccc4)c4sc5cc(-c6ccccc6)ccc5c34)c3c(c2)oc2ccccc23)cc1]

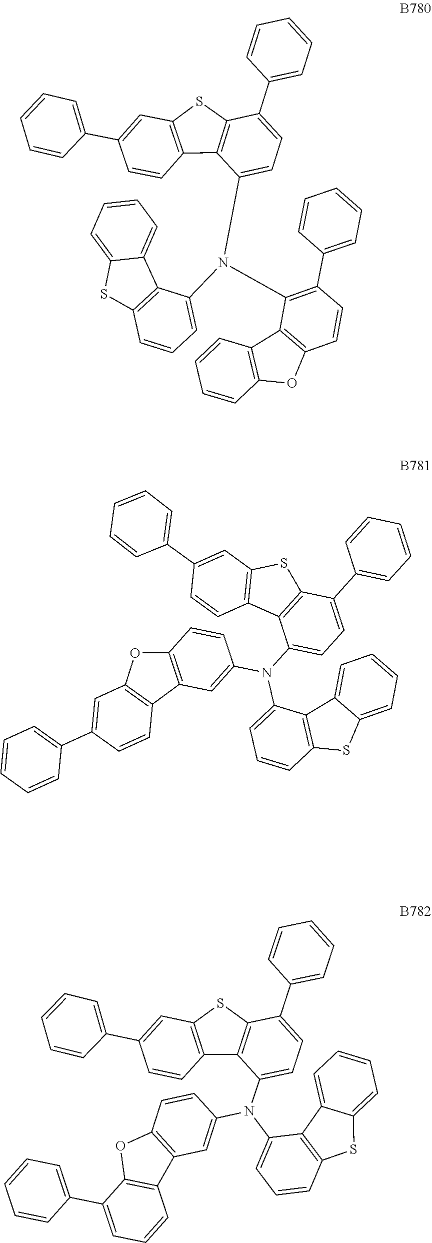 [SMILES: c1ccc(-c2ccc3c(c2)sc2c(-c4ccccc4)ccc(N(c4c(-c5ccccc5)ccc5oc6ccccc6c45)c4cccc5sc6ccccc6c45)c23)cc1 ; c1ccc(-c2ccc3c(c2)oc2ccc(N(c4cccc5sc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5sc6ccccc6c45)c23)cc1]

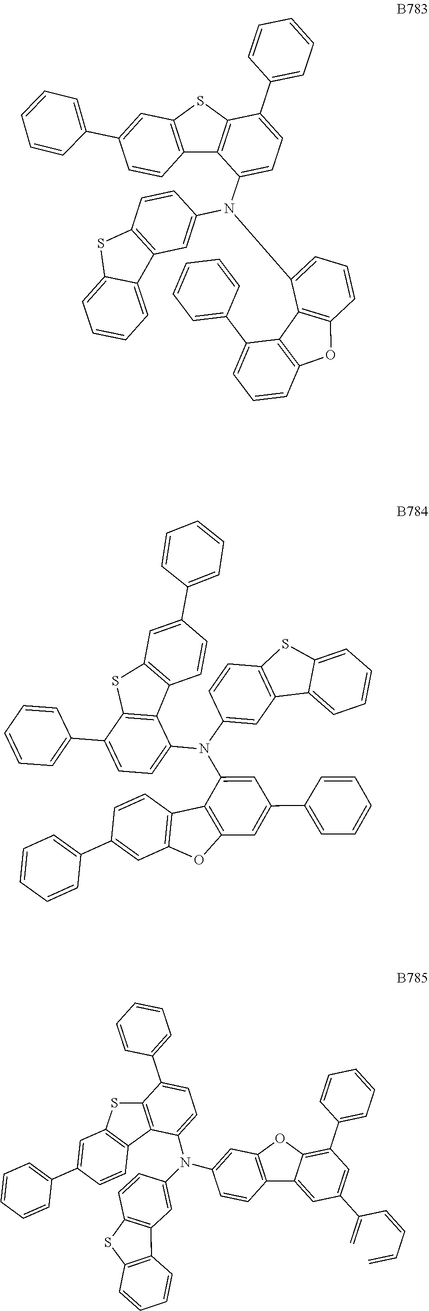 [SMILES: c1ccc(-c2ccc3c(c2)sc2c(-c4ccccc4)ccc(N(c4ccc5sc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5sc6ccccc6c5c4)c4ccc(-c5ccccc5)c5sc6cc(-c7ccccc7)ccc6c45)c23)cc1 ; C=C/C=C\C(=C)c1cc(-c2ccccc2)c2oc3cc(N(c4ccc5sc6ccccc6c5c4)c4ccc(-c5ccccc5)c5sc6cc(-c7ccccc7)ccc6c45)ccc3c2c1]

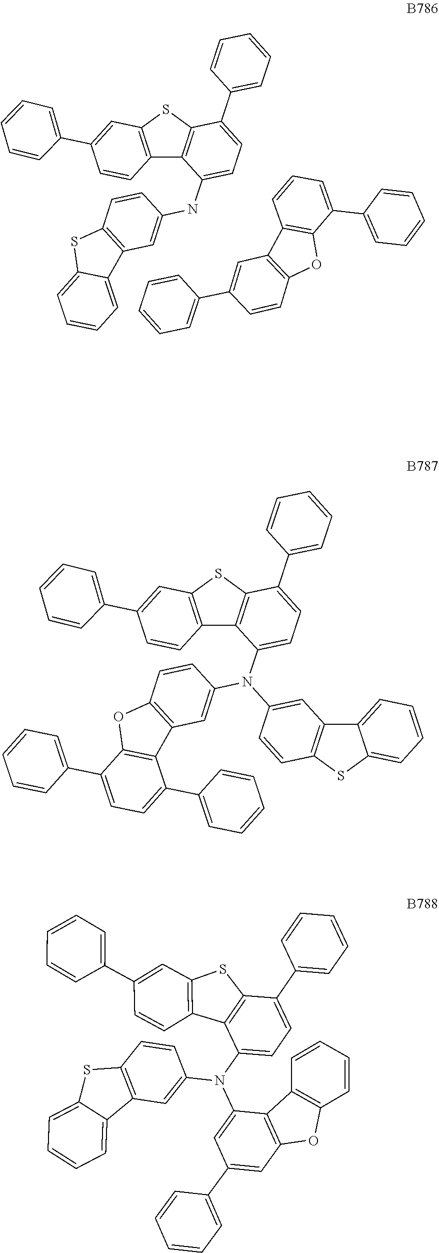

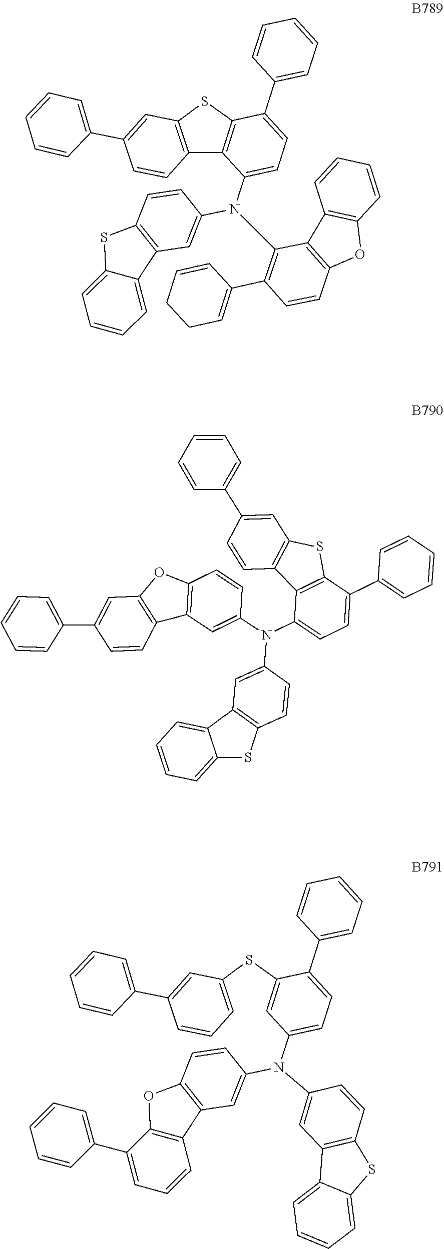 [SMILES: C1=CC(c2ccc3oc4ccccc4c3c2N(c2ccc3sc4ccccc4c3c2)c2ccc(-c3ccccc3)c3sc4cc(-c5ccccc5)ccc4c23)=CCC1 ; c1ccc(-c2ccc3c(c2)oc2ccc(N(c4ccc5sc6ccccc6c5c4)c4ccc(-c5ccccc5)c5sc6cc(-c7ccccc7)ccc6c45)cc23)cc1 ; c1ccc(-c2cccc(Sc3cc(N(c4ccc5oc6c(-c7ccccc7)cccc6c5c4)c4ccc5sc6ccccc6c5c4)ccc3-c3ccccc3)c2)cc1]

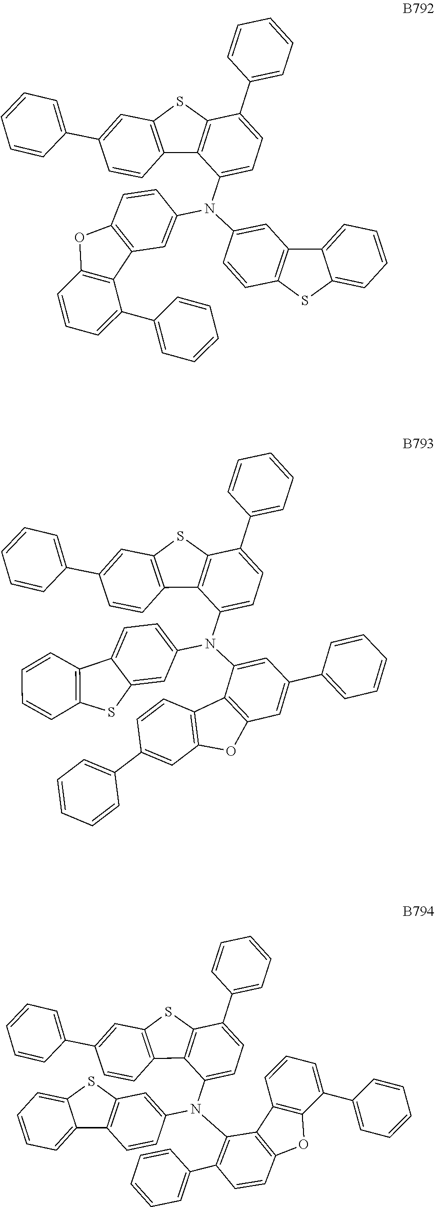 [SMILES: c1ccc(-c2ccc3c(c2)sc2c(-c4ccccc4)ccc(N(c4ccc5sc6ccccc6c5c4)c4ccc5oc6cccc(-c7ccccc7)c6c5c4)c23)cc1 ; c1ccc(-c2ccc3c(c2)oc2cc(-c4ccccc4)cc(N(c4ccc5c(c4)sc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4ccccc45)c4c(-c5ccccc5)ccc5oc6c(-c7ccccc7)cccc6c45)c23)cc1]

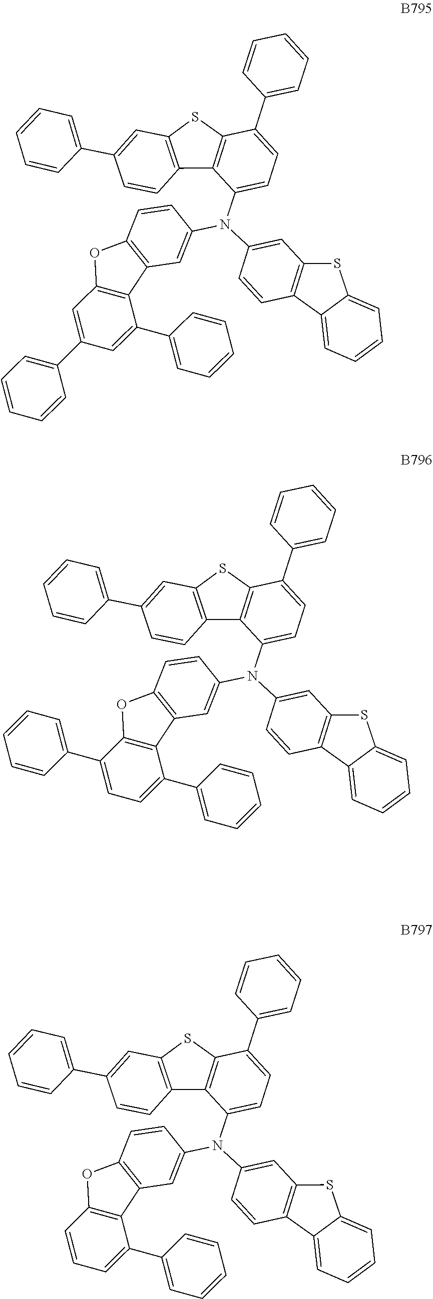 [SMILES: c1ccc(-c2cc(-c3ccccc3)c3c(c2)oc2ccc(N(c4ccc5c(c4)sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5c(c4)sc4ccccc45)c4ccc5oc6c(-c7ccccc7)ccc(-c7ccccc7)c6c5c4)c23)cc1 ; c1ccc(-c2ccc3c(c2)sc2c(-c4ccccc4)ccc(N(c4ccc5c(c4)sc4ccccc45)c4ccc5oc6cccc(-c7ccccc7)c6c5c4)c23)cc1]

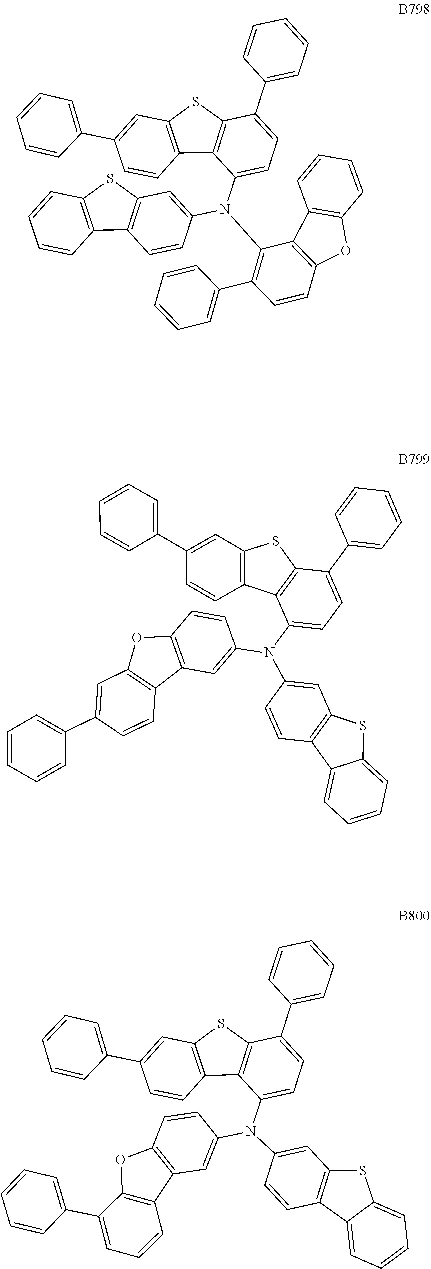 [SMILES: c1ccc(-c2ccc3c(c2)sc2c(-c4ccccc4)ccc(N(c4ccc5c(c4)sc4ccccc45)c4c(-c5ccccc5)ccc5oc6ccccc6c45)c23)cc1 ; c1ccc(-c2ccc3c(c2)oc2ccc(N(c4ccc5c(c4)sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5c(c4)sc4ccccc45)c4ccc5oc6c(-c7ccccc7)cccc6c5c4)c23)cc1]

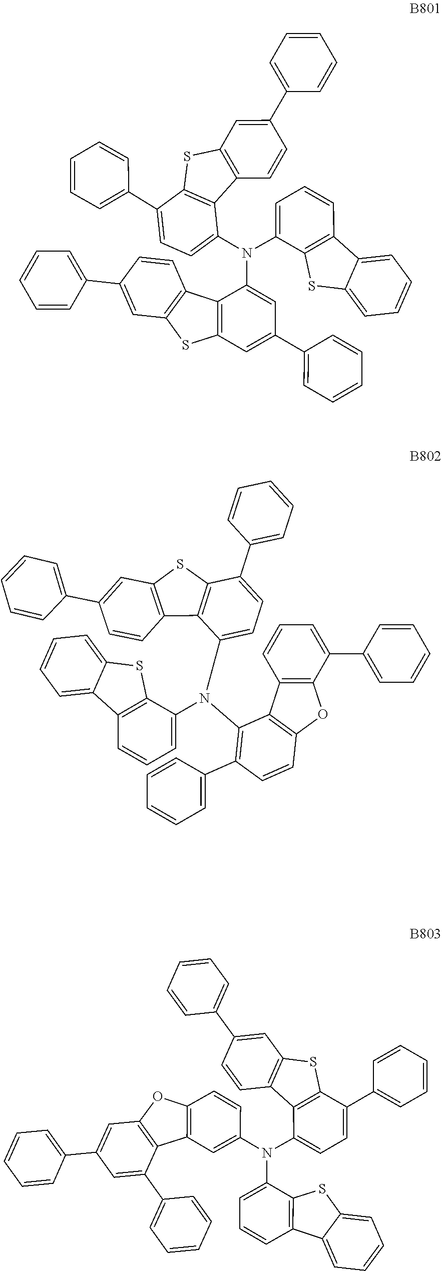 [SMILES: c1ccc(-c2ccc3c(c2)sc2cc(-c4ccccc4)cc(N(c4cccc5c4sc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5c4sc4ccccc45)c4c(-c5ccccc5)ccc5oc6c(-c7ccccc7)cccc6c45)c23)cc1 ; c1ccc(-c2cc(-c3ccccc3)c3c(c2)oc2ccc(N(c4cccc5c4sc4ccccc45)c4ccc(-c5ccccc5)c5sc6cc(-c7ccccc7)ccc6c45)cc23)cc1]

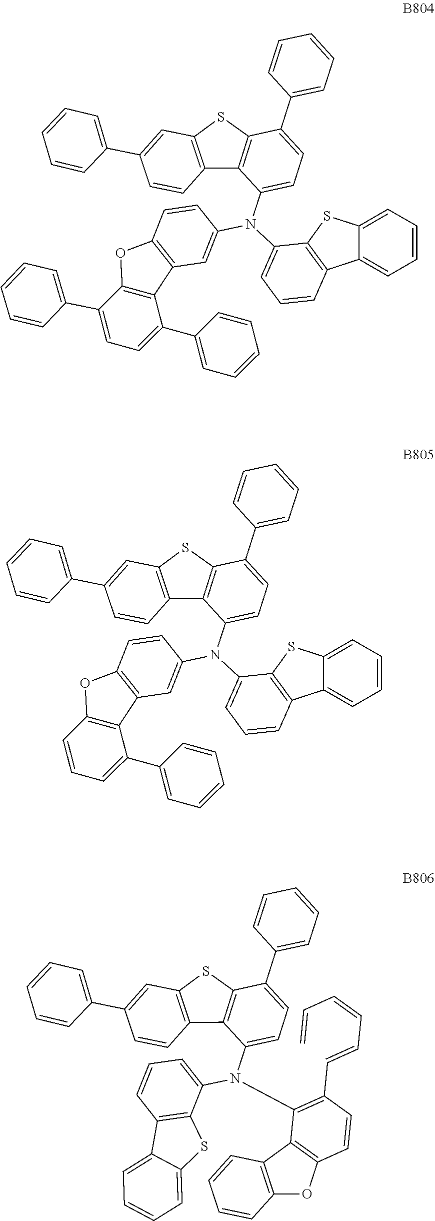 [SMILES: c1ccc(-c2ccc3c(c2)sc2c(-c4ccccc4)ccc(N(c4ccc5oc6c(-c7ccccc7)ccc(-c7ccccc7)c6c5c4)c4cccc5c4sc4ccccc45)c23)cc1 ; c1ccc(-c2ccc3c(c2)sc2c(-c4ccccc4)ccc(N(c4ccc5oc6cccc(-c7ccccc7)c6c5c4)c4cccc5c4sc4ccccc45)c23)cc1 ; C=C/C=C\C=C\c1ccc2oc3ccccc3c2c1N(c1cccc2c1sc1ccccc12)c1ccc(-c2ccccc2)c2sc3cc(-c4ccccc4)ccc3c12]

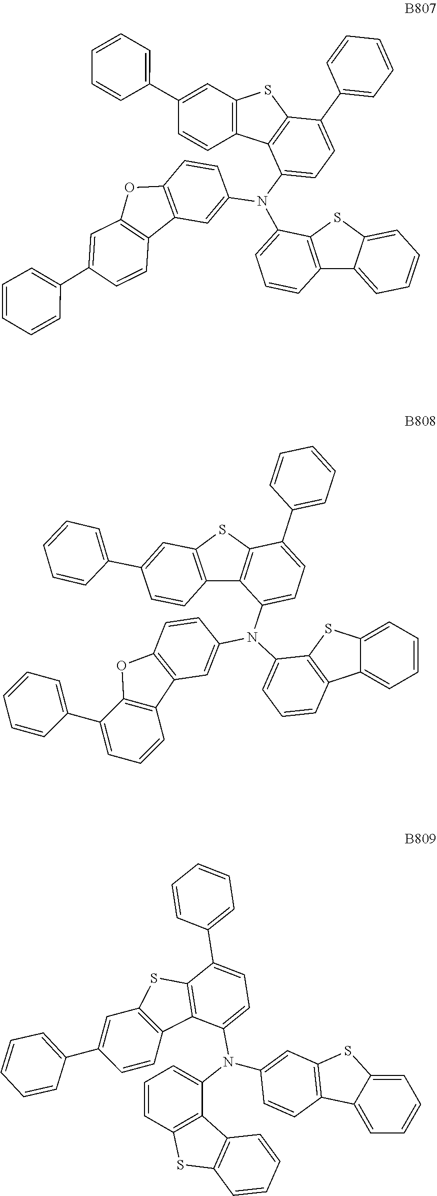 [SMILES: c1ccc(-c2ccc3c(c2)oc2ccc(N(c4cccc5c4sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5c4sc4ccccc45)c23)cc1 ; c1ccc(-c2ccc3c(c2)sc2c(-c4ccccc4)ccc(N(c4ccc5c(c4)sc4ccccc45)c4cccc5sc6ccccc6c45)c23)cc1]

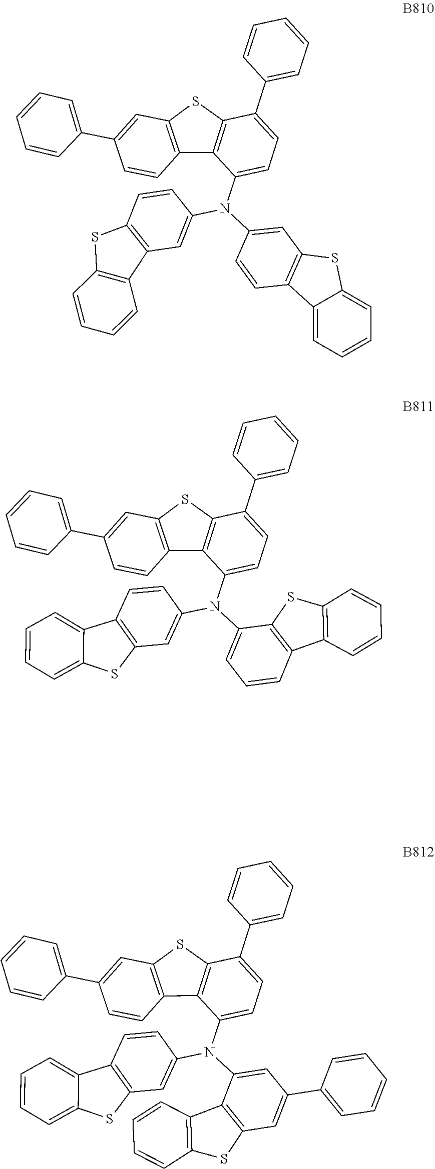 [SMILES: c1ccc(-c2ccc3c(c2)sc2c(-c4ccccc4)ccc(N(c4ccc5c(c4)sc4ccccc45)c4ccc5sc6ccccc6c5c4)c23)cc1 ; c1ccc(-c2ccc3c(c2)sc2c(-c4ccccc4)ccc(N(c4ccc5c(c4)sc4ccccc45)c4cccc5c4sc4ccccc45)c23)cc1 ; c1ccc(-c2ccc3c(c2)sc2c(-c4ccccc4)ccc(N(c4ccc5c(c4)sc4ccccc45)c4cc(-c5ccccc5)cc5sc6ccccc6c45)c23)cc1]

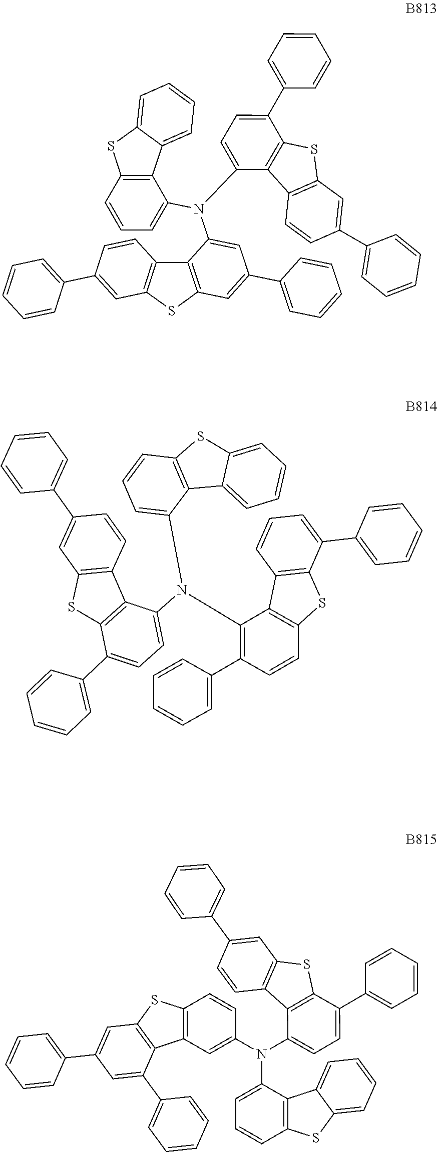 [SMILES: c1ccc(-c2ccc3c(c2)sc2cc(-c4ccccc4)cc(N(c4cccc5sc6ccccc6c45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5sc6ccccc6c45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5sc6ccccc6c45)c23)cc1]

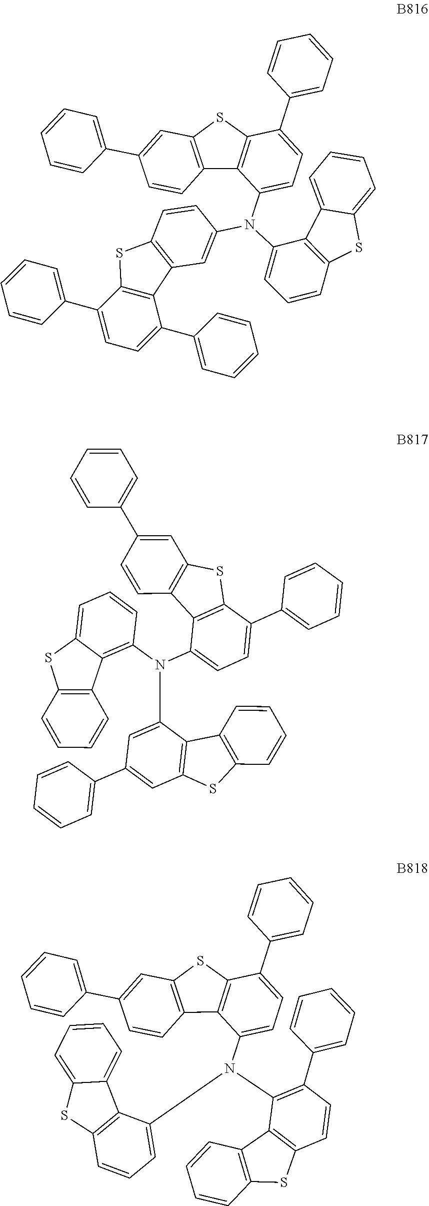 [SMILES: c1ccc(-c2ccc3c(c2)sc2c(-c4ccccc4)ccc(N(c4ccc5sc6c(-c7ccccc7)ccc(-c7ccccc7)c6c5c4)c4cccc5sc6ccccc6c45)c23)cc1 ; c1ccc(-c2ccc3c(c2)sc2c(-c4ccccc4)ccc(N(c4cccc5sc6ccccc6c45)c4cc(-c5ccccc5)cc5sc6ccccc6c45)c23)cc1 ; c1ccc(-c2ccc3c(c2)sc2c(-c4ccccc4)ccc(N(c4cccc5sc6ccccc6c45)c4c(-c5ccccc5)ccc5sc6ccccc6c45)c23)cc1]

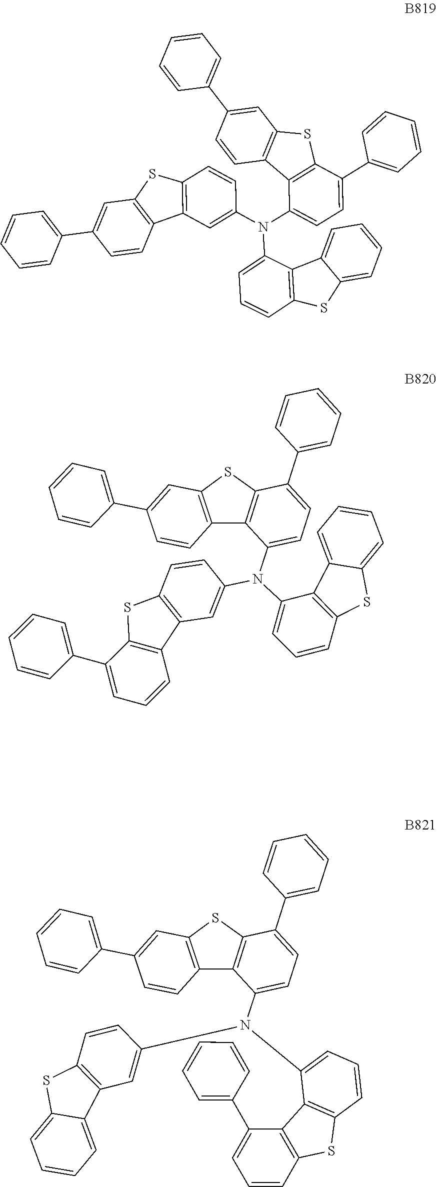 [SMILES: c1ccc(-c2ccc3c(c2)sc2ccc(N(c4cccc5sc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5sc6ccccc6c45)c23)cc1 ; c1ccc(-c2ccc3c(c2)sc2c(-c4ccccc4)ccc(N(c4ccc5sc6ccccc6c5c4)c4cccc5sc6cccc(-c7ccccc7)c6c45)c23)cc1]

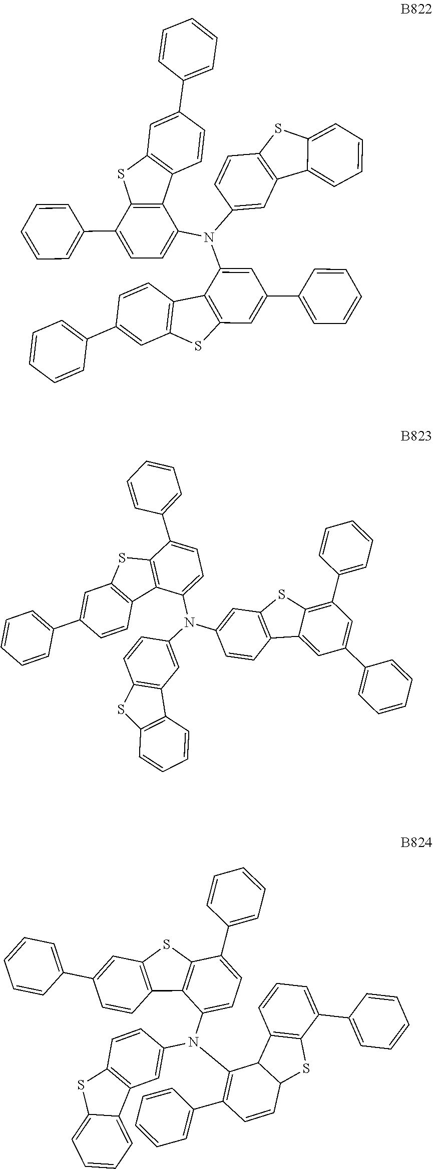 [SMILES: c1ccc(-c2ccc3c(c2)sc2cc(-c4ccccc4)cc(N(c4ccc5sc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4c(-c6ccccc6)cc(-c6ccccc6)cc45)c4ccc5sc6ccccc6c5c4)c23)cc1 ; C1=CC2Sc3c(-c4ccccc4)cccc3C2C(N(c2ccc3sc4ccccc4c3c2)c2ccc(-c3ccccc3)c3sc4cc(-c5ccccc5)ccc4c23)=C1c1ccccc1]

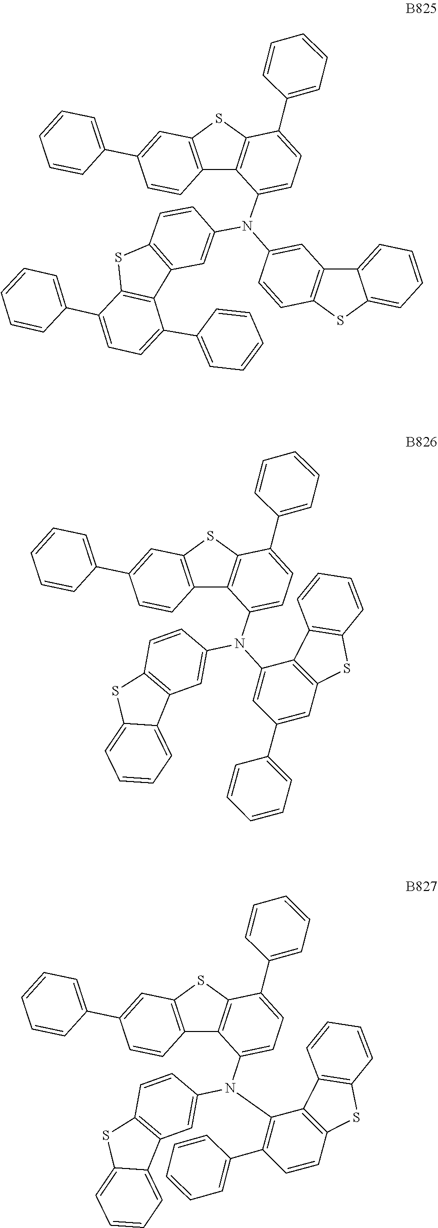

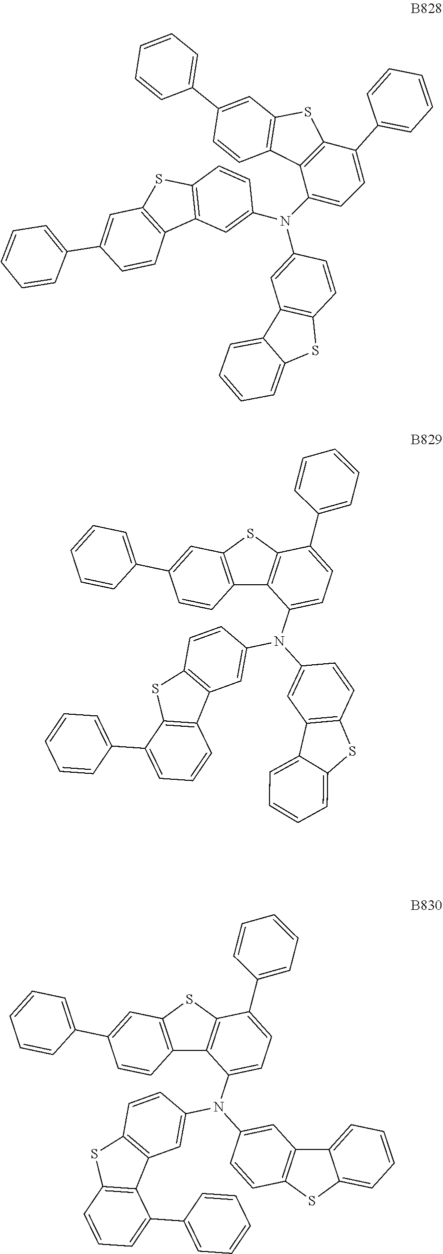 [SMILES: c1ccc(-c2ccc3c(c2)sc2ccc(N(c4ccc5sc6ccccc6c5c4)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)sc2c(-c4ccccc4)ccc(N(c4ccc5sc6ccccc6c5c4)c4ccc5sc6cccc(-c7ccccc7)c6c5c4)c23)cc1]

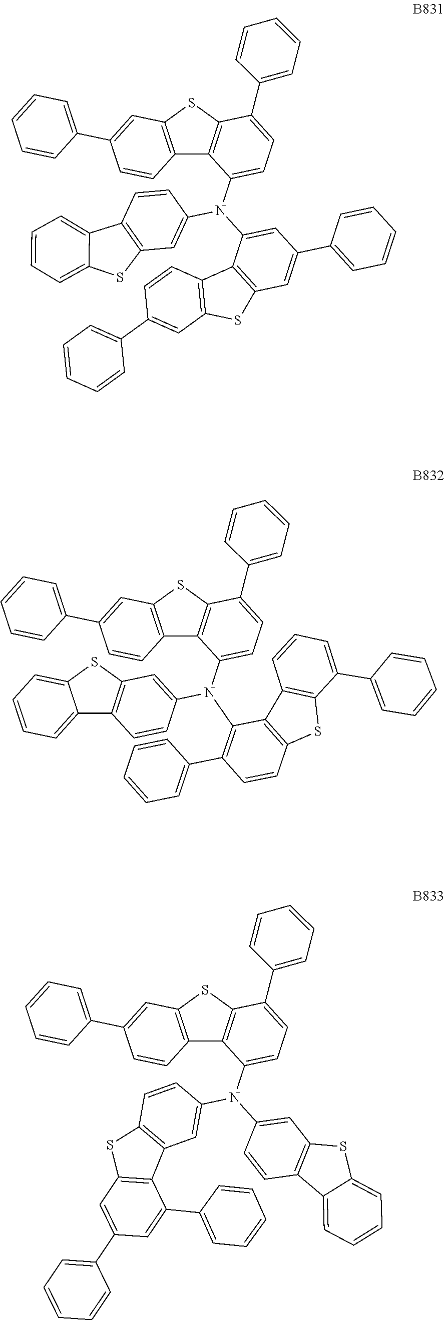 [SMILES: c1ccc(-c2ccc3c(c2)sc2cc(-c4ccccc4)cc(N(c4ccc5c(c4)sc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5c(c4)sc4ccccc45)c4ccc5sc6cc(-c7ccccc7)cc(-c7ccccc7)c6c5c4)c23)cc1]

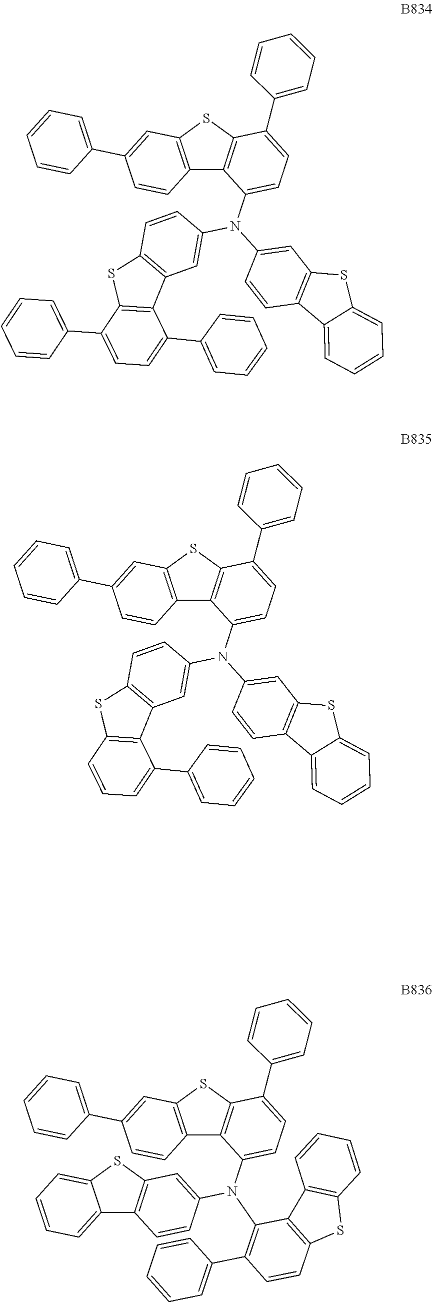 [SMILES: c1ccc(-c2ccc3c(c2)sc2c(-c4ccccc4)ccc(N(c4ccc5c(c4)sc4ccccc45)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)sc2c(-c4ccccc4)ccc(N(c4ccc5c(c4)sc4ccccc45)c4c(-c5ccccc5)ccc5sc6ccccc6c45)c23)cc1]

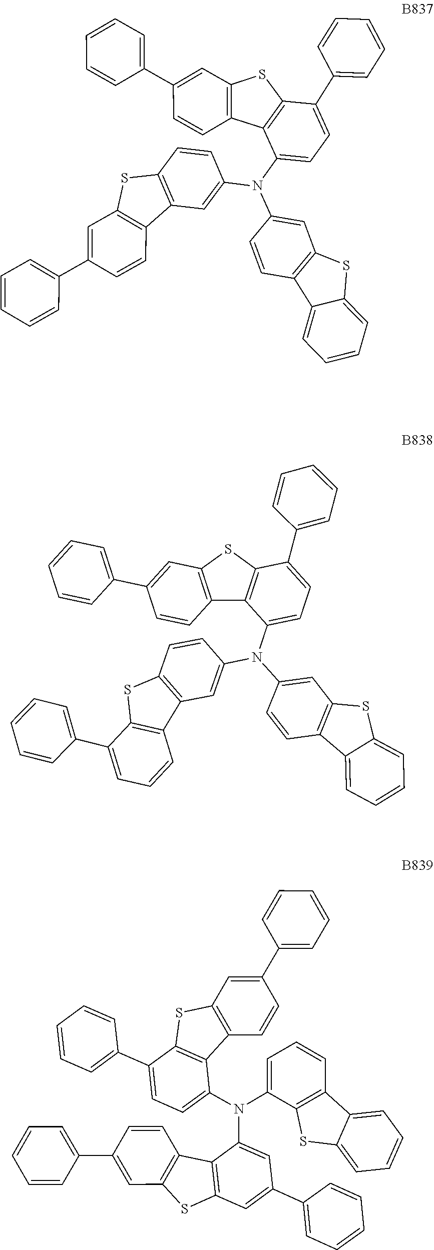 [SMILES: c1ccc(-c2ccc3c(c2)sc2ccc(N(c4ccc5c(c4)sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)sc2cc(-c4ccccc4)cc(N(c4cccc5c4sc4ccccc45)c4ccc(-c5ccccc5)c5sc6cc(-c7ccccc7)ccc6c45)c23)cc1]

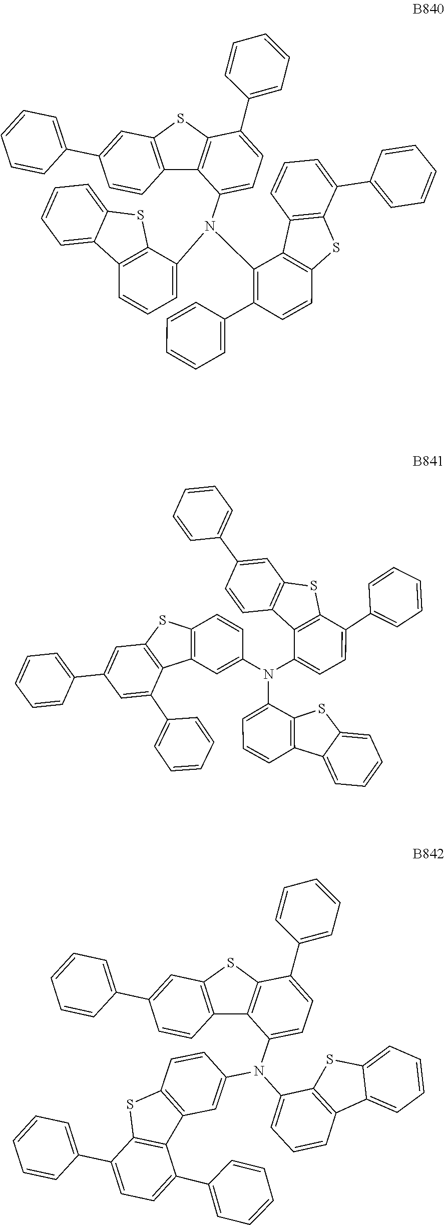 [SMILES: c1ccc(-c2ccc3c(c2)sc2c(-c4ccccc4)ccc(N(c4cccc5c4sc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5c4sc4ccccc45)c23)cc1]

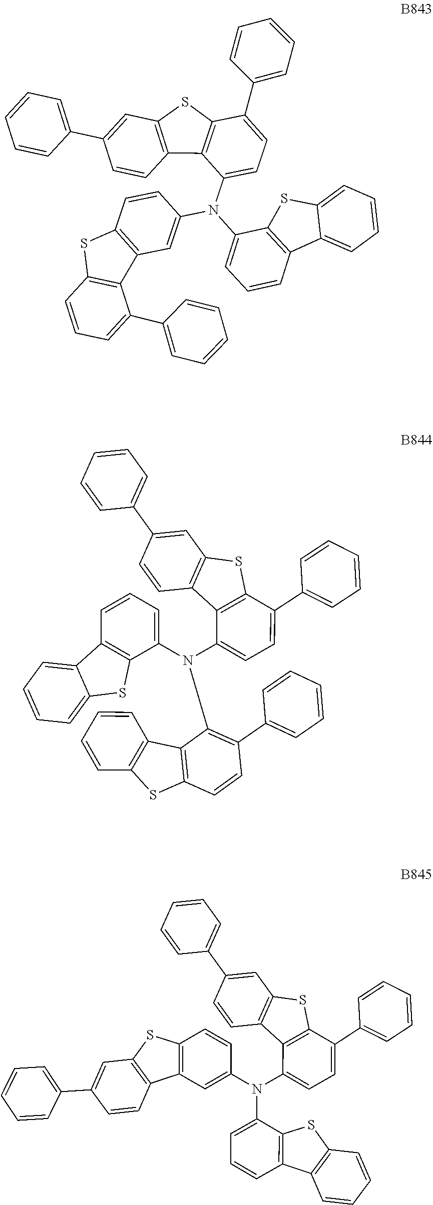 [SMILES: c1ccc(-c2ccc3c(c2)sc2c(-c4ccccc4)ccc(N(c4ccc5sc6cccc(-c7ccccc7)c6c5c4)c4cccc5c4sc4ccccc45)c23)cc1 ; c1ccc(-c2ccc3c(c2)sc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)sc2ccc(N(c4cccc5c4sc4ccccc45)c4ccc(-c5ccccc5)c5sc6cc(-c7ccccc7)ccc6c45)cc23)cc1]

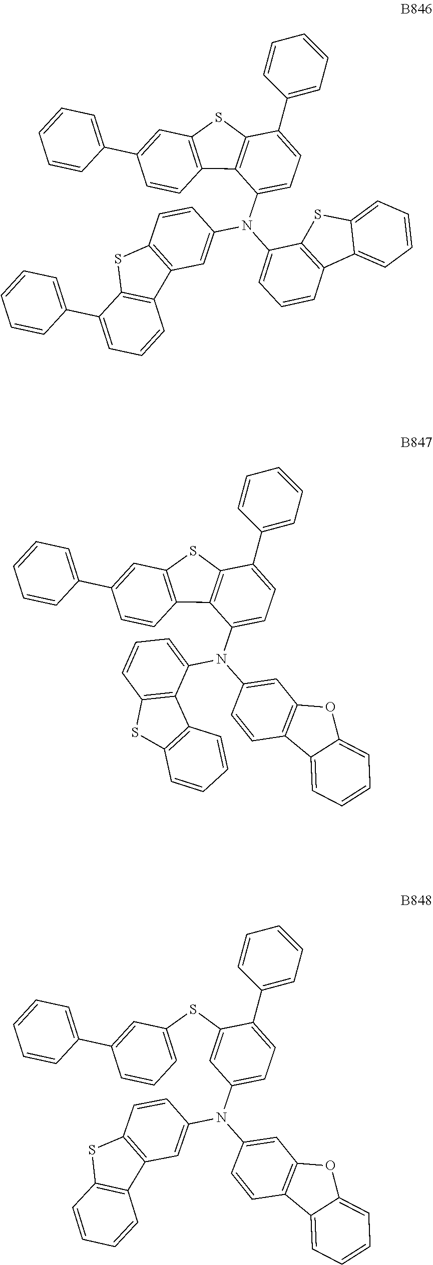 [SMILES: c1ccc(-c2ccc3c(c2)sc2c(-c4ccccc4)ccc(N(c4ccc5sc6c(-c7ccccc7)cccc6c5c4)c4cccc5c4sc4ccccc45)c23)cc1 ; c1ccc(-c2ccc3c(c2)sc2c(-c4ccccc4)ccc(N(c4ccc5c(c4)oc4ccccc45)c4cccc5sc6ccccc6c45)c23)cc1 ; c1ccc(-c2cccc(Sc3cc(N(c4ccc5c(c4)oc4ccccc45)c4ccc5sc6ccccc6c5c4)ccc3-c3ccccc3)c2)cc1]

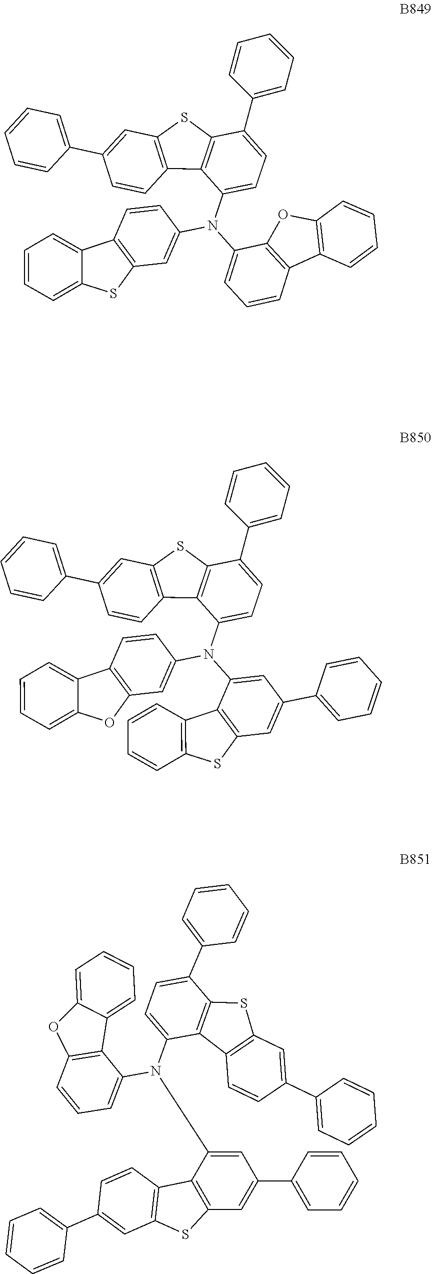 [SMILES: c1ccc(-c2ccc3c(c2)sc2c(-c4ccccc4)ccc(N(c4ccc5c(c4)sc4ccccc45)c4cccc5c4oc4ccccc45)c23)cc1 ; c1ccc(-c2ccc3c(c2)sc2c(-c4ccccc4)ccc(N(c4ccc5c(c4)oc4ccccc45)c4cc(-c5ccccc5)cc5sc6ccccc6c45)c23)cc1 ; c1ccc(-c2ccc3c(c2)sc2cc(-c4ccccc4)cc(N(c4cccc5oc6ccccc6c45)c4ccc(-c5ccccc5)c5sc6cc(-c7ccccc7)ccc6c45)c23)cc1]

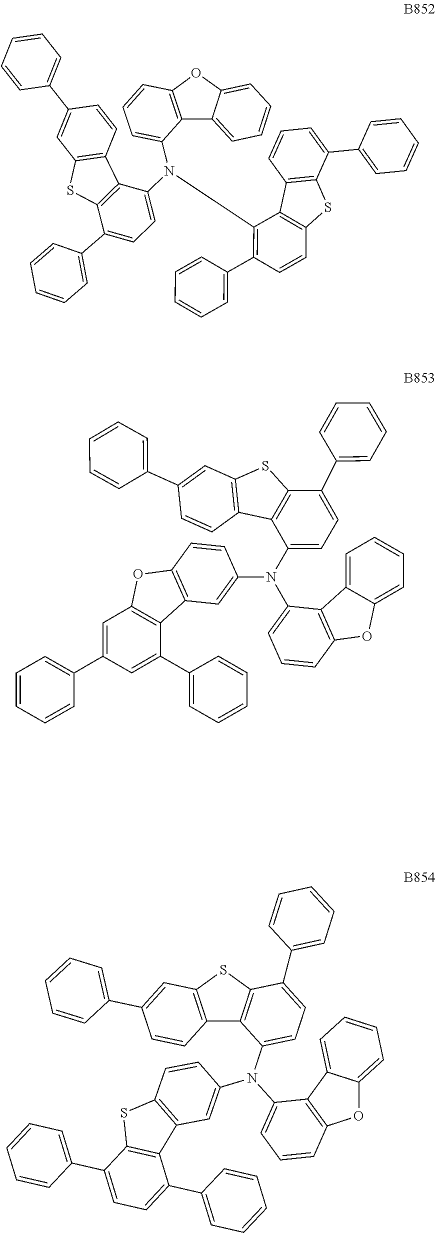 [SMILES: c1ccc(-c2ccc3c(c2)sc2c(-c4ccccc4)ccc(N(c4cccc5oc6ccccc6c45)c4c(-c5ccccc5)ccc5sc6c(-c7ccccc7)cccc6c45)c23)cc1 ; c1ccc(-c2cc(-c3ccccc3)c3c(c2)oc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5oc6ccccc6c45)c23)cc1]

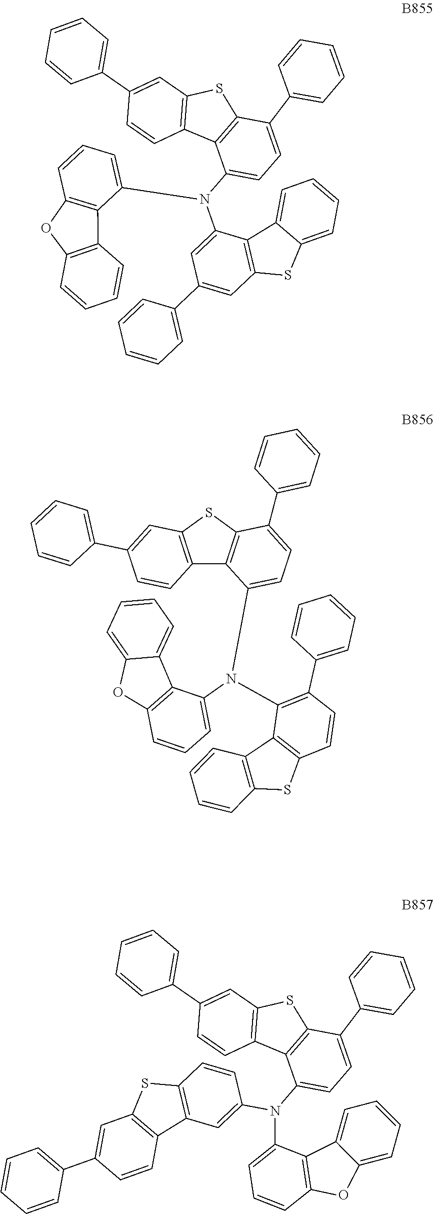 [SMILES: c1ccc(-c2ccc3c(c2)sc2c(-c4ccccc4)ccc(N(c4cccc5oc6ccccc6c45)c4cc(-c5ccccc5)cc5sc6ccccc6c45)c23)cc1 ; c1ccc(-c2ccc3c(c2)sc2c(-c4ccccc4)ccc(N(c4cccc5oc6ccccc6c45)c4c(-c5ccccc5)ccc5sc6ccccc6c45)c23)cc1 ; c1ccc(-c2ccc3c(c2)sc2ccc(N(c4cccc5oc6ccccc6c45)c4ccc(-c5ccccc5)c5sc6cc(-c7ccccc7)ccc6c45)cc23)cc1]

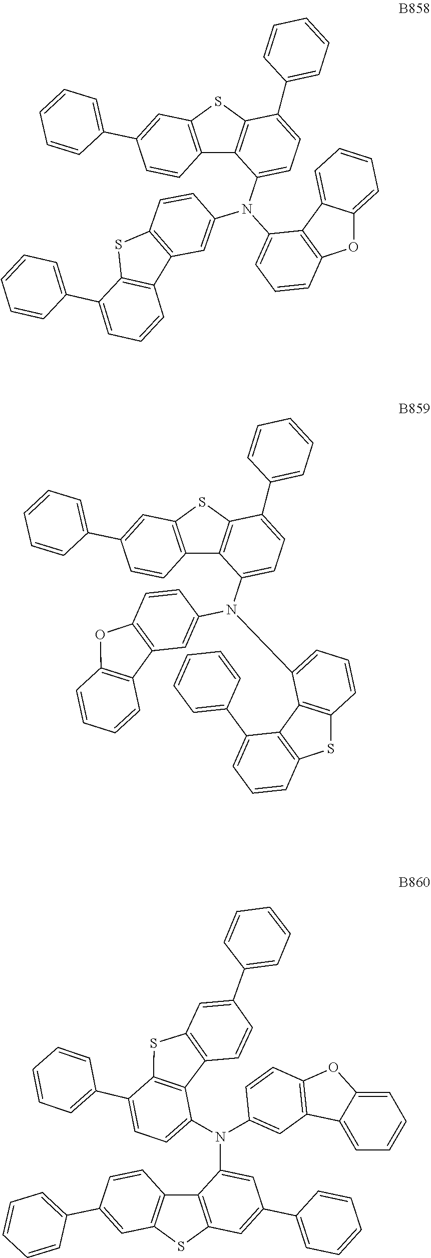 [SMILES: c1ccc(-c2ccc3c(c2)sc2c(-c4ccccc4)ccc(N(c4ccc5sc6c(-c7ccccc7)cccc6c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4cccc5sc6cccc(-c7ccccc7)c6c45)c23)cc1 ; c1ccc(-c2ccc3c(c2)sc2cc(-c4ccccc4)cc(N(c4ccc5oc6ccccc6c5c4)c4ccc(-c5ccccc5)c5sc6cc(-c7ccccc7)ccc6c45)c23)cc1]

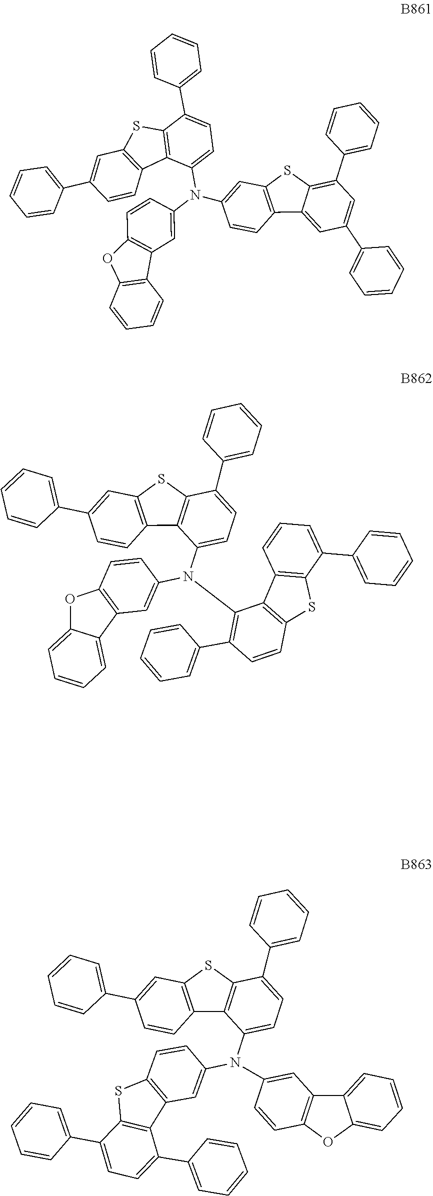 [SMILES: c1ccc(-c2ccc3c(c2)sc2c(-c4ccccc4)ccc(N(c4ccc5c(c4)sc4c(-c6ccccc6)cc(-c6ccccc6)cc45)c4ccc5oc6ccccc6c5c4)c23)cc1 ; c1ccc(-c2ccc3c(c2)sc2c(-c4ccccc4)ccc(N(c4ccc5oc6ccccc6c5c4)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5oc6ccccc6c5c4)c4ccc5sc6c(-c7ccccc7)ccc(-c7ccccc7)c6c5c4)c23)cc1]

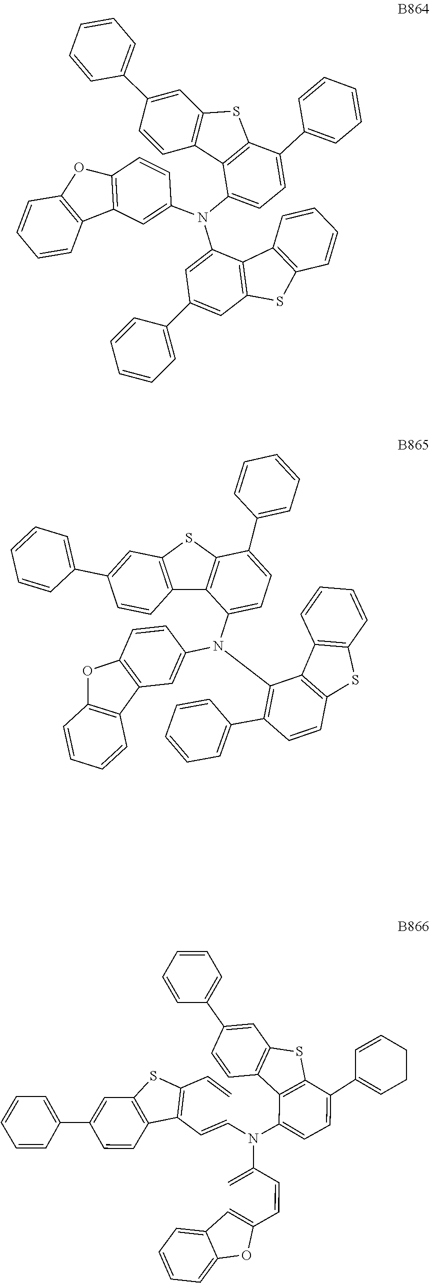 [SMILES: c1ccc(-c2ccc3c(c2)sc2c(-c4ccccc4)ccc(N(c4ccc5oc6ccccc6c5c4)c4cc(-c5ccccc5)cc5sc6ccccc6c45)c23)cc1 ; c1ccc(-c2ccc3c(c2)sc2c(-c4ccccc4)ccc(N(c4ccc5oc6ccccc6c5c4)c4c(-c5ccccc5)ccc5sc6ccccc6c45)c23)cc1 ; C=Cc1sc2cc(-c3ccccc3)ccc2c1/C=C/N(C(=C)/C=C\c1cc2ccccc2o1)c1ccc(C2=CCCC=C2)c2sc3cc(-c4ccccc4)ccc3c12]

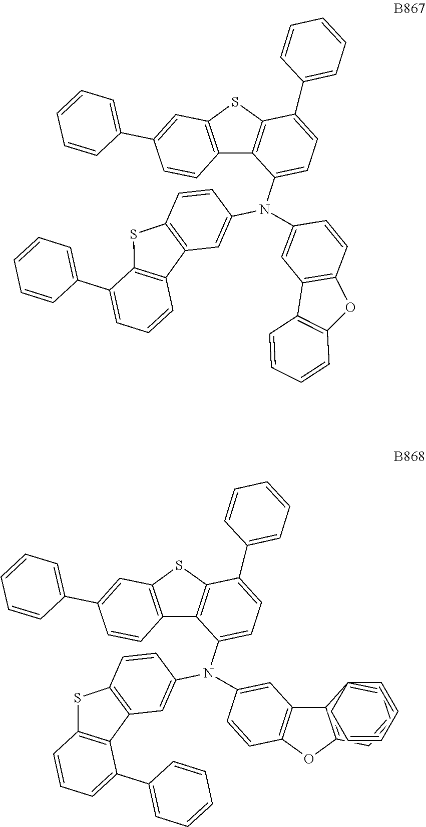

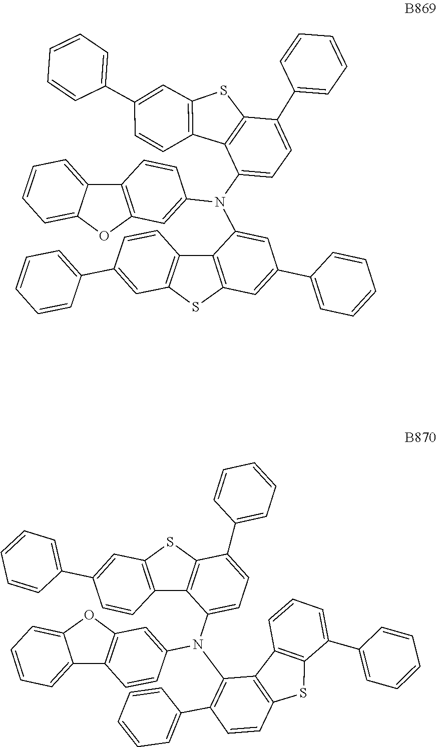 [SMILES: c1ccc(-c2ccc3c(c2)sc2cc(-c4ccccc4)cc(N(c4ccc5c(c4)oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)oc4ccccc45)c4c(-c5ccccc5)ccc5sc6c(-c7ccccc7)cccc6c45)c23)cc1]

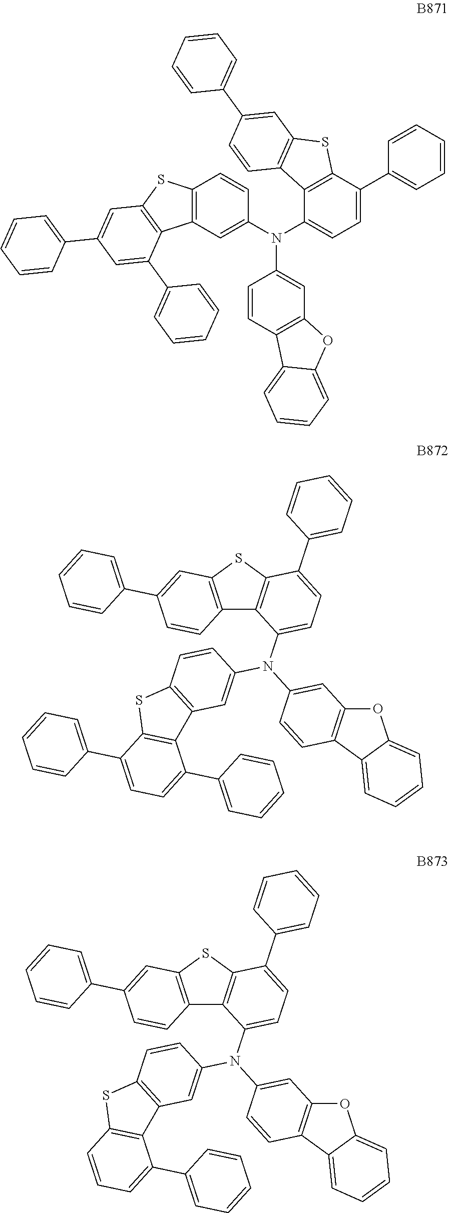 [SMILES: c1ccc(-c2ccc3c(c2)sc2c(-c4ccccc4)ccc(N(c4ccc5c(c4)oc4ccccc45)c4ccc5sc6cc(-c7ccccc7)cc(-c7ccccc7)c6c5c4)c23)cc1 ; c1ccc(-c2ccc3c(c2)sc2c(-c4ccccc4)ccc(N(c4ccc5c(c4)oc4ccccc45)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5c(c4)oc4ccccc45)c4ccc5sc6cccc(-c7ccccc7)c6c5c4)c23)cc1]

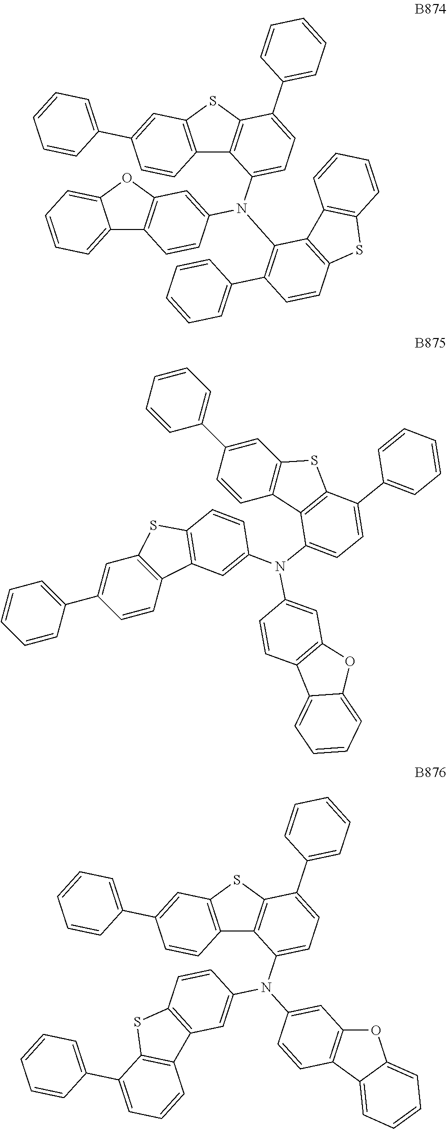

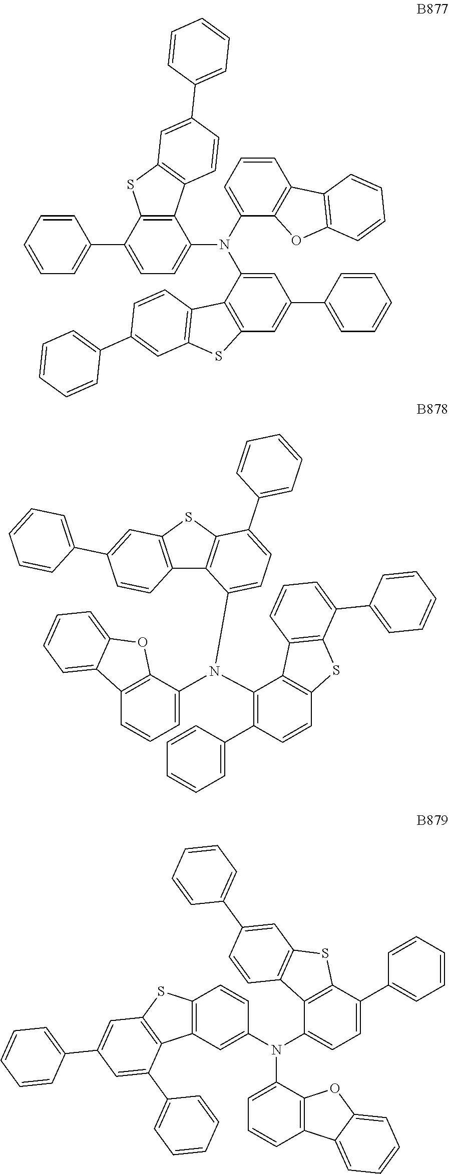 [SMILES: c1ccc(-c2ccc3c(c2)sc2cc(-c4ccccc4)cc(N(c4cccc5c4oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5c4oc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5c4oc4ccccc45)c23)cc1]

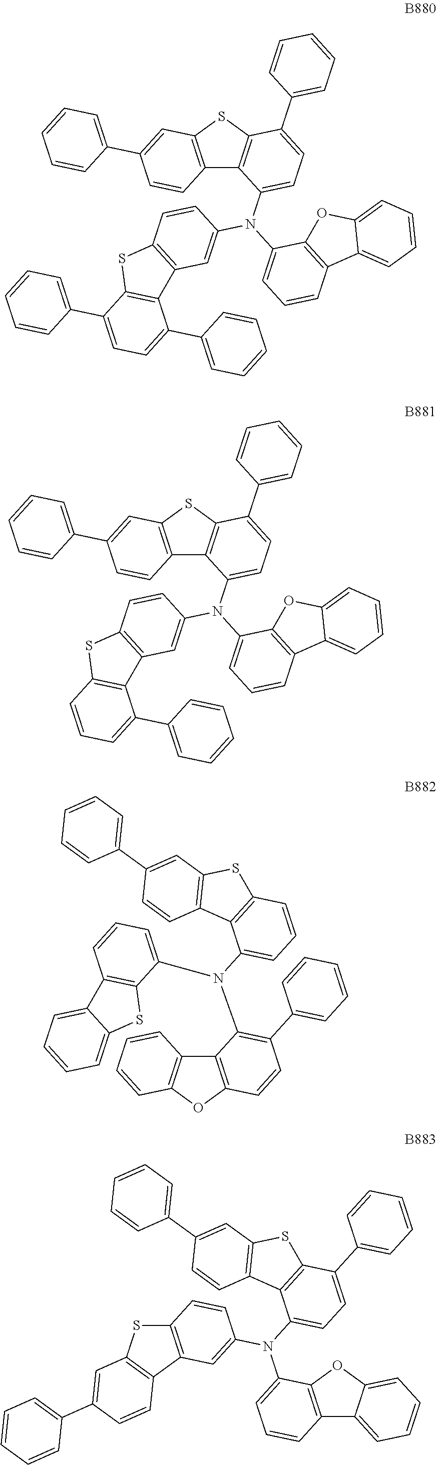 [SMILES: c1ccc(-c2ccc3c(c2)sc2c(-c4ccccc4)ccc(N(c4ccc5sc6c(-c7ccccc7)ccc(-c7ccccc7)c6c5c4)c4cccc5c4oc4ccccc45)c23)cc1 ; c1ccc(-c2ccc3c(c2)sc2c(-c4ccccc4)ccc(N(c4ccc5sc6cccc(-c7ccccc7)c6c5c4)c4cccc5c4oc4ccccc45)c23)cc1 ; c1ccc(-c2ccc3c(c2)sc2cccc(N(c4cccc5c4sc4ccccc45)c4c(-c5ccccc5)ccc5oc6ccccc6c45)c23)cc1 ; c1ccc(-c2ccc3c(c2)sc2ccc(N(c4cccc5c4oc4ccccc45)c4ccc(-c5ccccc5)c5sc6cc(-c7ccccc7)ccc6c45)cc23)cc1]

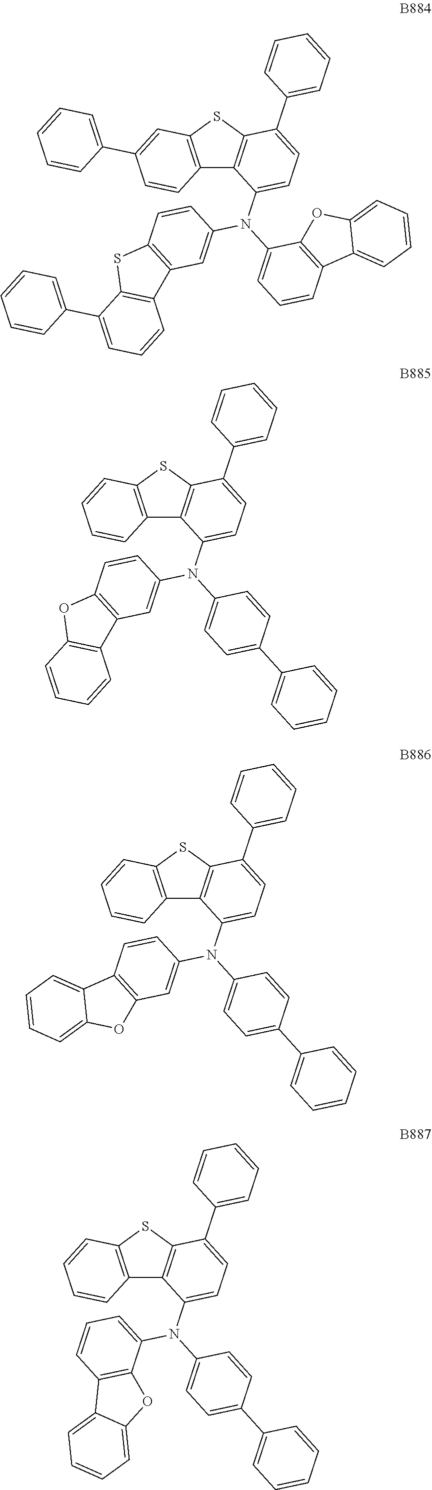

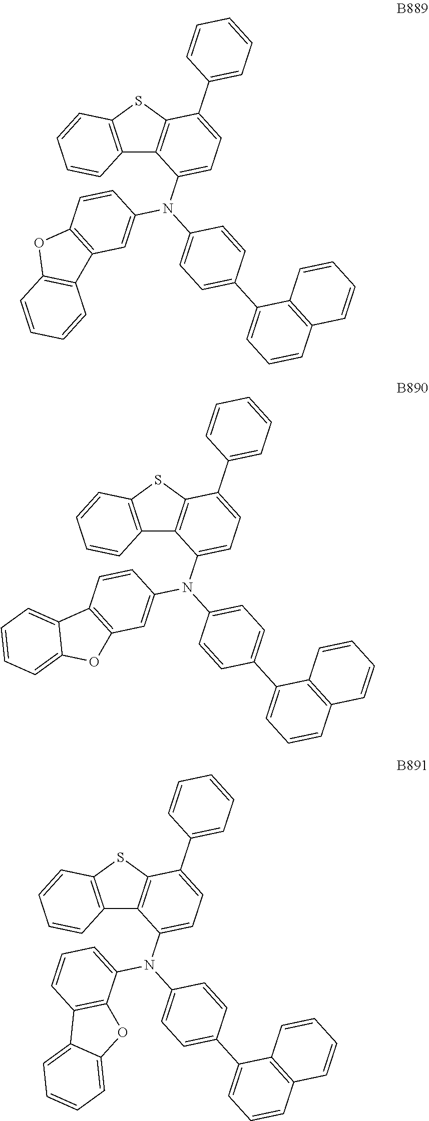

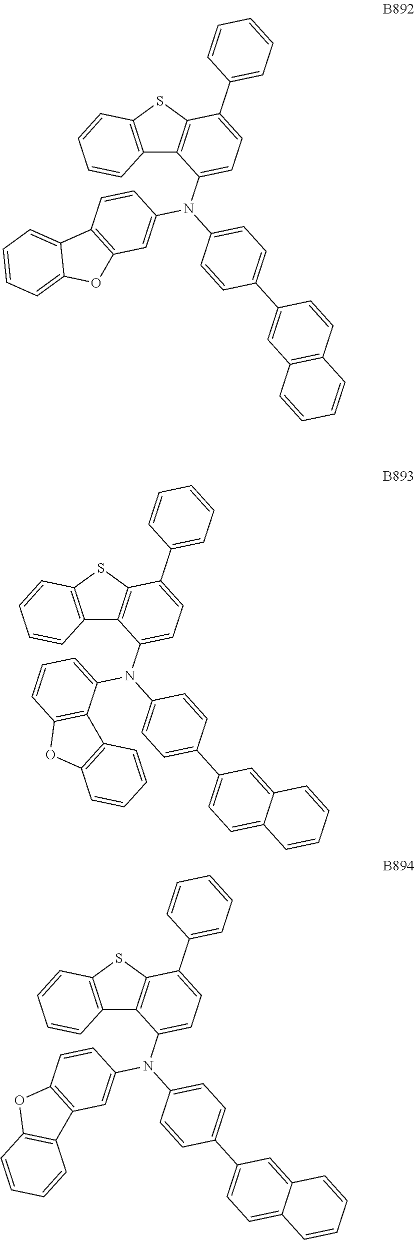 [SMILES: c1ccc(-c2ccc(N(c3ccc(-c4ccc5ccccc5c4)cc3)c3ccc4c(c3)oc3ccccc34)c3c2sc2ccccc23)cc1 ; c1ccc(-c2ccc(N(c3ccc(-c4ccc5ccccc5c4)cc3)c3cccc4oc5ccccc5c34)c3c2sc2ccccc23)cc1 ; c1ccc(-c2ccc(N(c3ccc(-c4ccc5ccccc5c4)cc3)c3ccc4oc5ccccc5c4c3)c3c2sc2ccccc23)cc1]

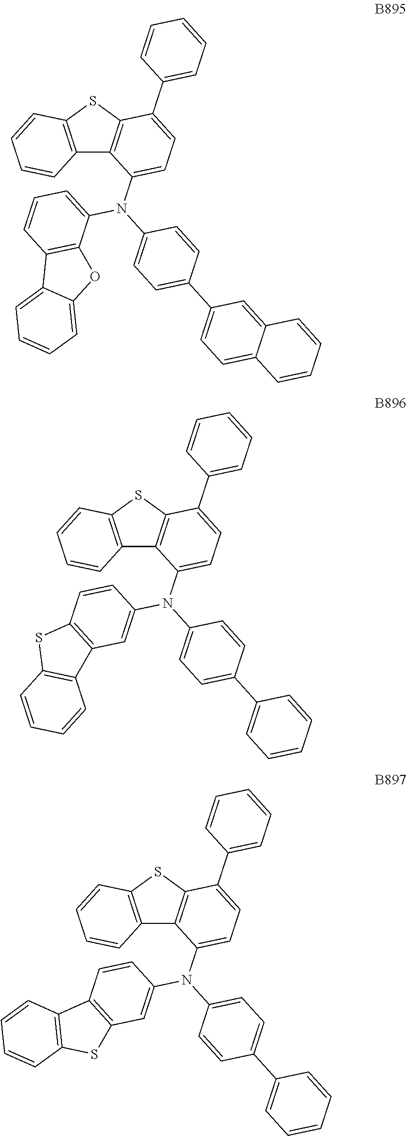

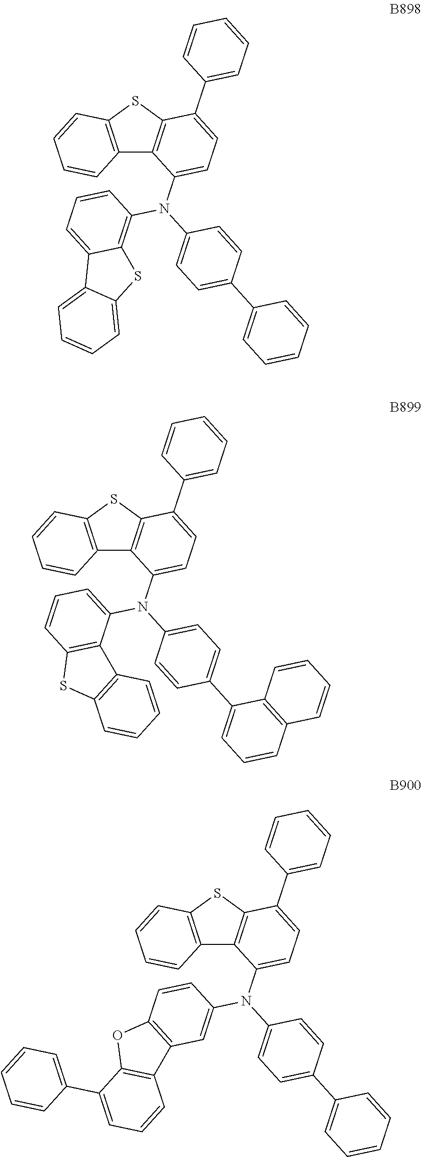 [SMILES: c1ccc(-c2ccc(N(c3cccc4c3sc3ccccc34)c3ccc(-c4ccccc4)c4sc5ccccc5c34)cc2)cc1 ; c1ccc(-c2ccc(N(c3ccc(-c4cccc5ccccc45)cc3)c3cccc4sc5ccccc5c34)c3c2sc2ccccc23)cc1 ; c1ccc(-c2ccc(N(c3ccc4oc5c(-c6ccccc6)cccc5c4c3)c3ccc(-c4ccccc4)c4sc5ccccc5c34)cc2)cc1]

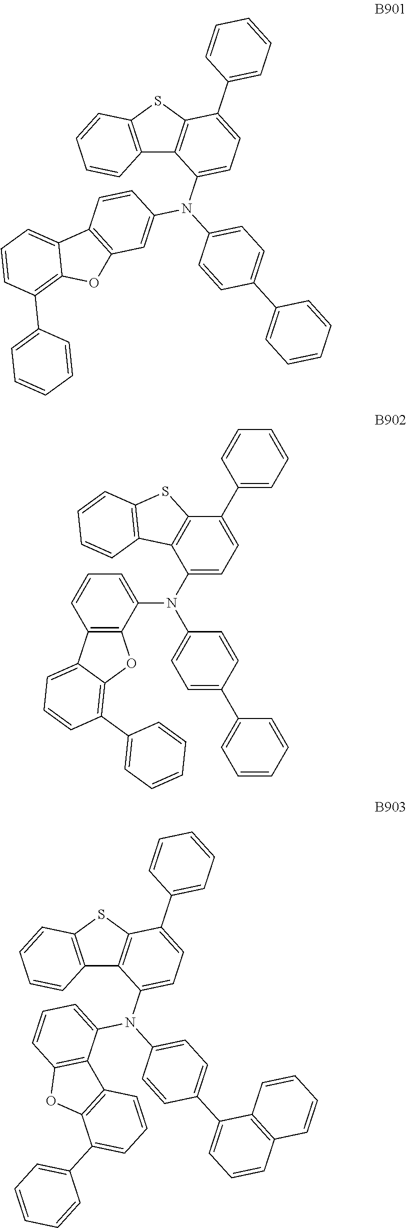 [SMILES: c1ccc(-c2ccc(N(c3ccc4c(c3)oc3c(-c5ccccc5)cccc34)c3ccc(-c4ccccc4)c4sc5ccccc5c34)cc2)cc1 ; c1ccc(-c2ccc(N(c3cccc4c3oc3c(-c5ccccc5)cccc34)c3ccc(-c4ccccc4)c4sc5ccccc5c34)cc2)cc1 ; c1ccc(-c2cccc3c2oc2cccc(N(c4ccc(-c5cccc6ccccc56)cc4)c4ccc(-c5ccccc5)c5sc6ccccc6c45)c23)cc1]

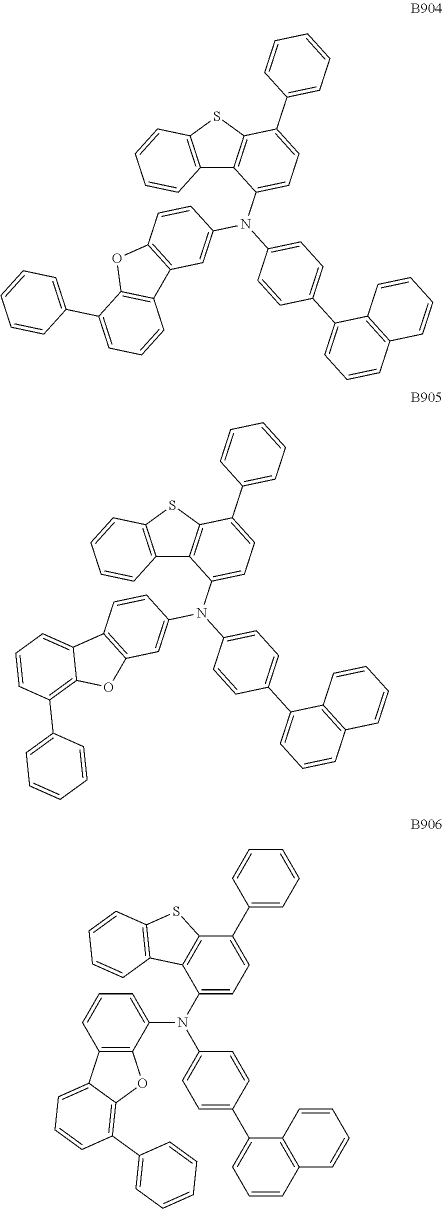 [SMILES: c1ccc(-c2cccc3c2oc2ccc(N(c4ccc(-c5cccc6ccccc56)cc4)c4ccc(-c5ccccc5)c5sc6ccccc6c45)cc23)cc1 ; c1ccc(-c2cccc3c2oc2cc(N(c4ccc(-c5cccc6ccccc56)cc4)c4ccc(-c5ccccc5)c5sc6ccccc6c45)ccc23)cc1 ; c1ccc(-c2cccc3c2oc2c(N(c4ccc(-c5cccc6ccccc56)cc4)c4ccc(-c5ccccc5)c5sc6ccccc6c45)cccc23)cc1]

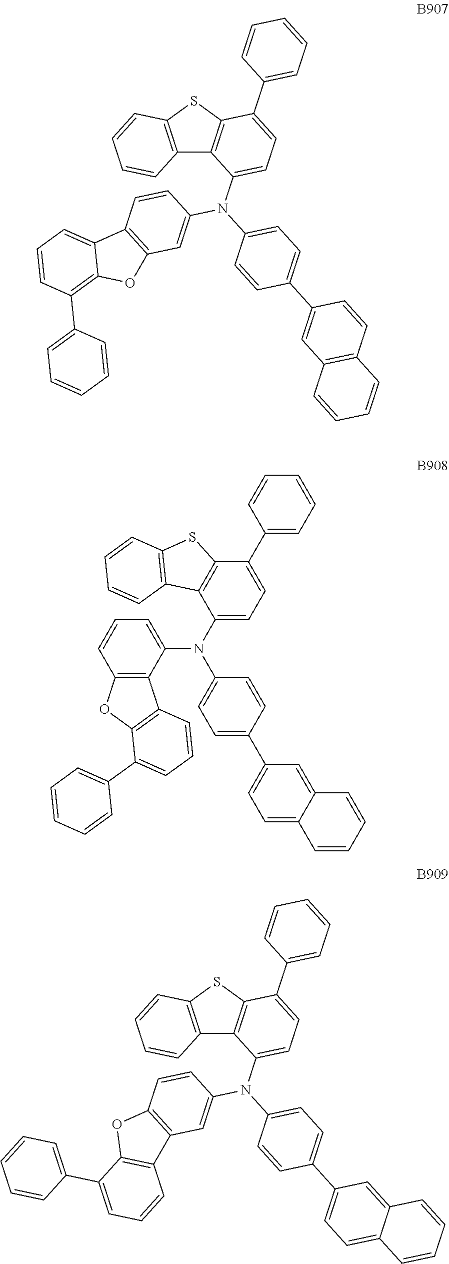 [SMILES: c1ccc(-c2cccc3c2oc2cc(N(c4ccc(-c5ccc6ccccc6c5)cc4)c4ccc(-c5ccccc5)c5sc6ccccc6c45)ccc23)cc1 ; c1ccc(-c2cccc3c2oc2cccc(N(c4ccc(-c5ccc6ccccc6c5)cc4)c4ccc(-c5ccccc5)c5sc6ccccc6c45)c23)cc1 ; c1ccc(-c2cccc3c2oc2ccc(N(c4ccc(-c5ccc6ccccc6c5)cc4)c4ccc(-c5ccccc5)c5sc6ccccc6c45)cc23)cc1]

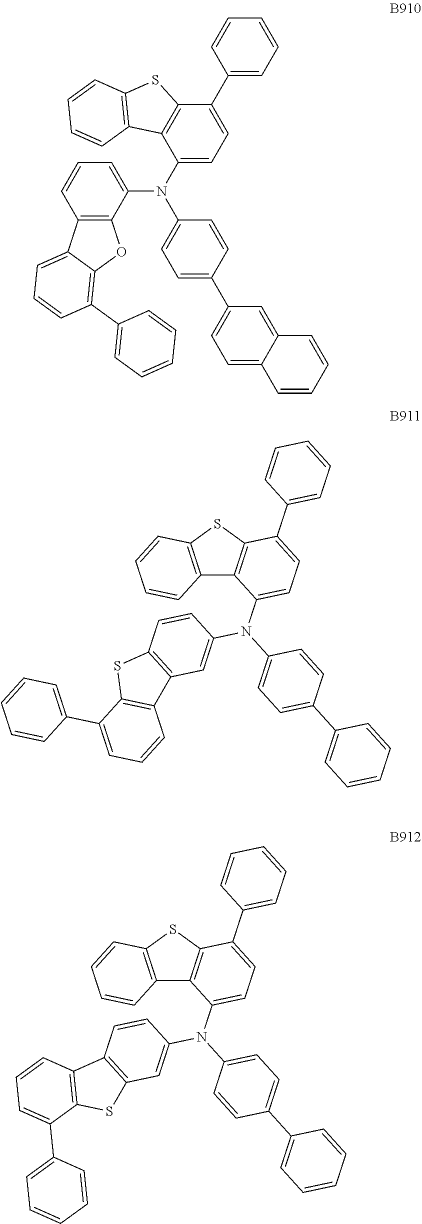 [SMILES: c1ccc(-c2cccc3c2oc2c(N(c4ccc(-c5ccc6ccccc6c5)cc4)c4ccc(-c5ccccc5)c5sc6ccccc6c45)cccc23)cc1 ; c1ccc(-c2ccc(N(c3ccc4sc5c(-c6ccccc6)cccc5c4c3)c3ccc(-c4ccccc4)c4sc5ccccc5c34)cc2)cc1 ; c1ccc(-c2ccc(N(c3ccc4c(c3)sc3c(-c5ccccc5)cccc34)c3ccc(-c4ccccc4)c4sc5ccccc5c34)cc2)cc1]

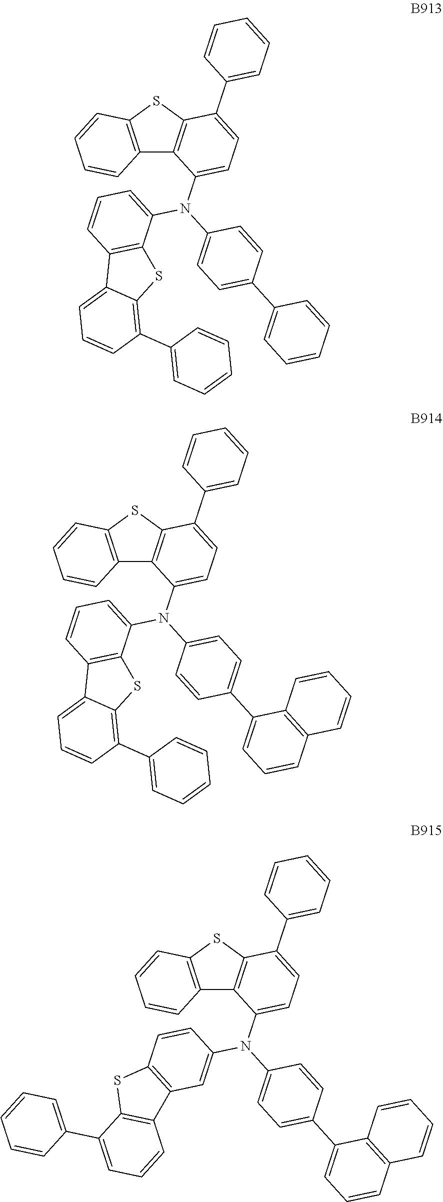

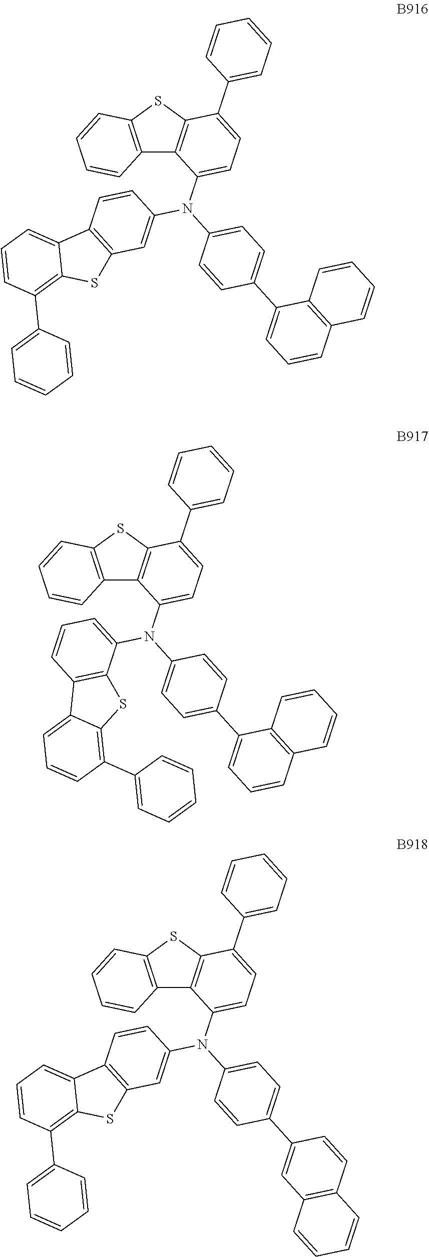 [SMILES: c1ccc(-c2cccc3c2sc2cc(N(c4ccc(-c5cccc6ccccc56)cc4)c4ccc(-c5ccccc5)c5sc6ccccc6c45)ccc23)cc1 ; c1ccc(-c2cccc3c2sc2c(N(c4ccc(-c5cccc6ccccc56)cc4)c4ccc(-c5ccccc5)c5sc6ccccc6c45)cccc23)cc1 ; c1ccc(-c2cccc3c2sc2cc(N(c4ccc(-c5ccc6ccccc6c5)cc4)c4ccc(-c5ccccc5)c5sc6ccccc6c45)ccc23)cc1]

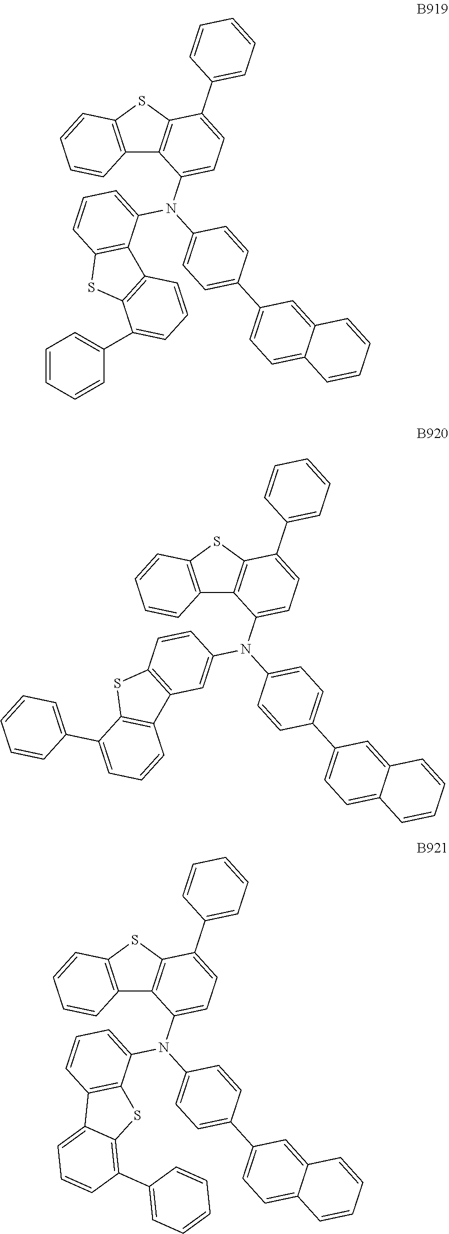 [SMILES: c1ccc(-c2cccc3c2sc2cccc(N(c4ccc(-c5ccc6ccccc6c5)cc4)c4ccc(-c5ccccc5)c5sc6ccccc6c45)c23)cc1 ; c1ccc(-c2cccc3c2sc2ccc(N(c4ccc(-c5ccc6ccccc6c5)cc4)c4ccc(-c5ccccc5)c5sc6ccccc6c45)cc23)cc1 ; c1ccc(-c2cccc3c2sc2c(N(c4ccc(-c5ccc6ccccc6c5)cc4)c4ccc(-c5ccccc5)c5sc6ccccc6c45)cccc23)cc1]

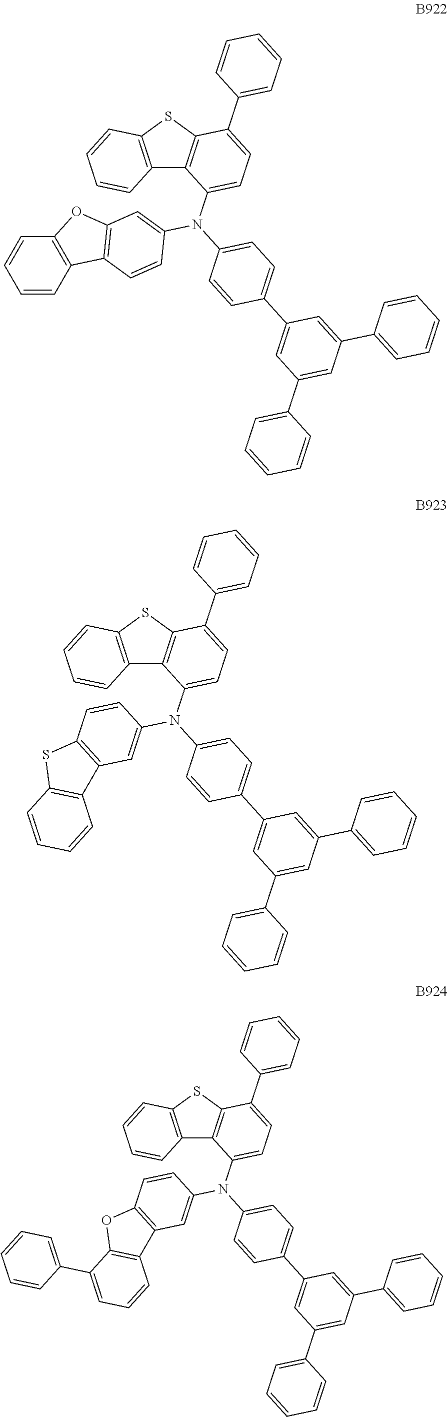 [SMILES: c1ccc(-c2cc(-c3ccccc3)cc(-c3ccc(N(c4ccc5c(c4)oc4ccccc45)c4ccc(-c5ccccc5)c5sc6ccccc6c45)cc3)c2)cc1 ; c1ccc(-c2cc(-c3ccccc3)cc(-c3ccc(N(c4ccc5sc6ccccc6c5c4)c4ccc(-c5ccccc5)c5sc6ccccc6c45)cc3)c2)cc1 ; c1ccc(-c2cc(-c3ccccc3)cc(-c3ccc(N(c4ccc5oc6c(-c7ccccc7)cccc6c5c4)c4ccc(-c5ccccc5)c5sc6ccccc6c45)cc3)c2)cc1]

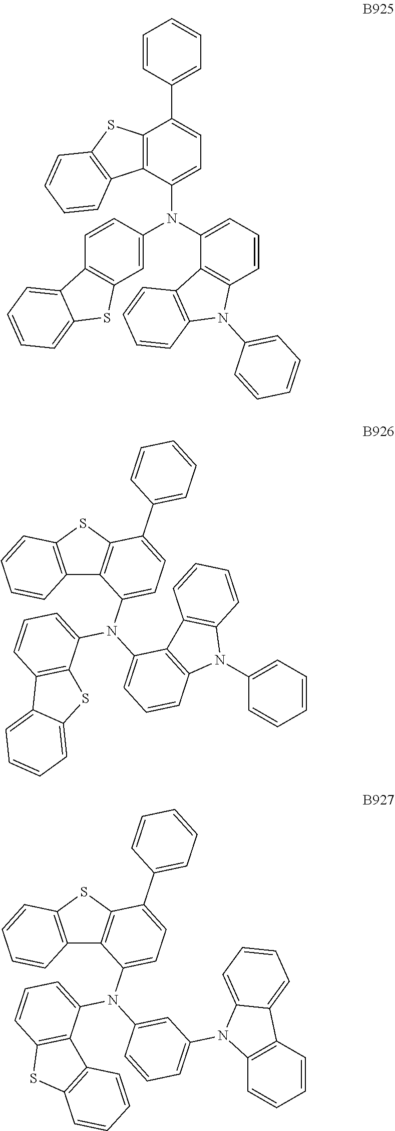 [SMILES: c1ccc(-c2ccc(N(c3ccc4c(c3)sc3ccccc34)c3cccc4c3c3ccccc3n4-c3ccccc3)c3c2sc2ccccc23)cc1 ; c1ccc(-c2ccc(N(c3cccc4c3sc3ccccc34)c3cccc4c3c3ccccc3n4-c3ccccc3)c3c2sc2ccccc23)cc1 ; c1ccc(-c2ccc(N(c3cccc(-n4c5ccccc5c5ccccc54)c3)c3cccc4sc5ccccc5c34)c3c2sc2ccccc23)cc1]

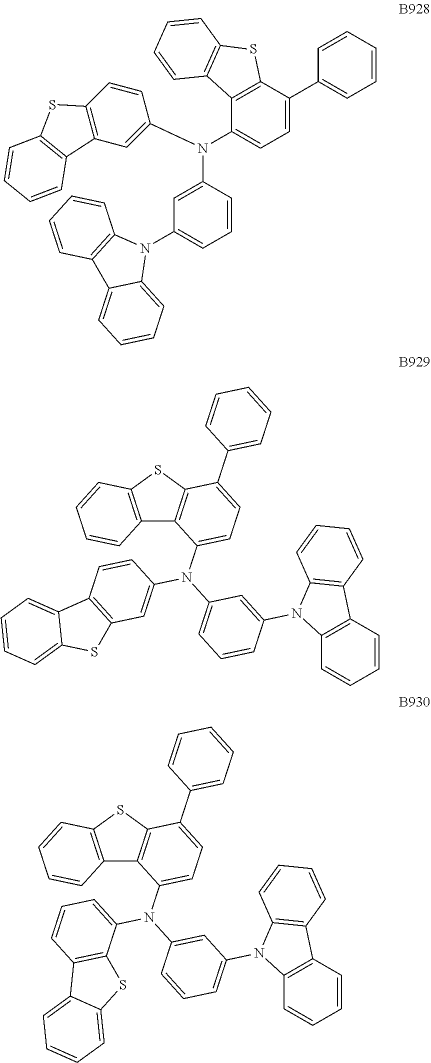 [SMILES: c1ccc(-c2ccc(N(c3cccc(-n4c5ccccc5c5ccccc54)c3)c3ccc4sc5ccccc5c4c3)c3c2sc2ccccc23)cc1 ; c1ccc(-c2ccc(N(c3cccc(-n4c5ccccc5c5ccccc54)c3)c3ccc4c(c3)sc3ccccc34)c3c2sc2ccccc23)cc1 ; c1ccc(-c2ccc(N(c3cccc(-n4c5ccccc5c5ccccc54)c3)c3cccc4c3sc3ccccc34)c3c2sc2ccccc23)cc1]

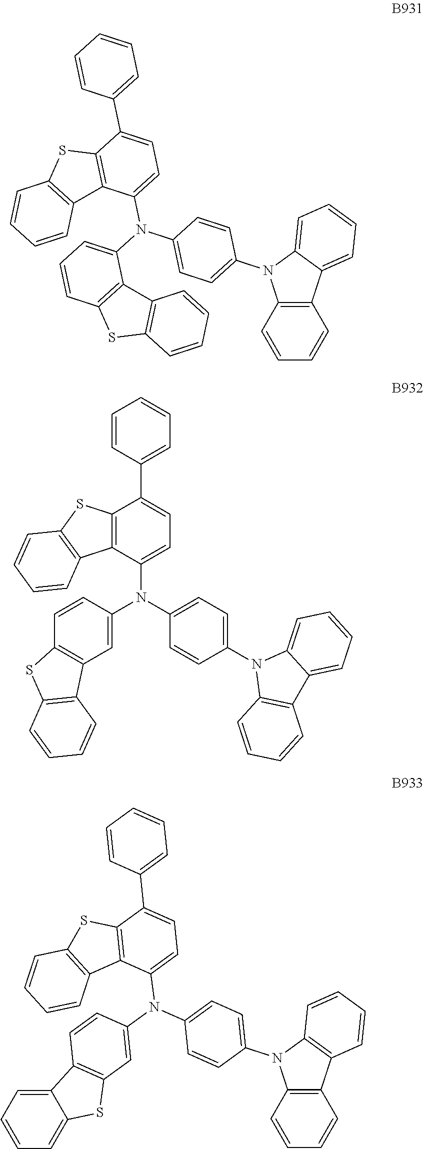 [SMILES: c1ccc(-c2ccc(N(c3ccc(-n4c5ccccc5c5ccccc54)cc3)c3cccc4sc5ccccc5c34)c3c2sc2ccccc23)cc1 ; c1ccc(-c2ccc(N(c3ccc(-n4c5ccccc5c5ccccc54)cc3)c3ccc4sc5ccccc5c4c3)c3c2sc2ccccc23)cc1 ; c1ccc(-c2ccc(N(c3ccc(-n4c5ccccc5c5ccccc54)cc3)c3ccc4c(c3)sc3ccccc34)c3c2sc2ccccc23)cc1]

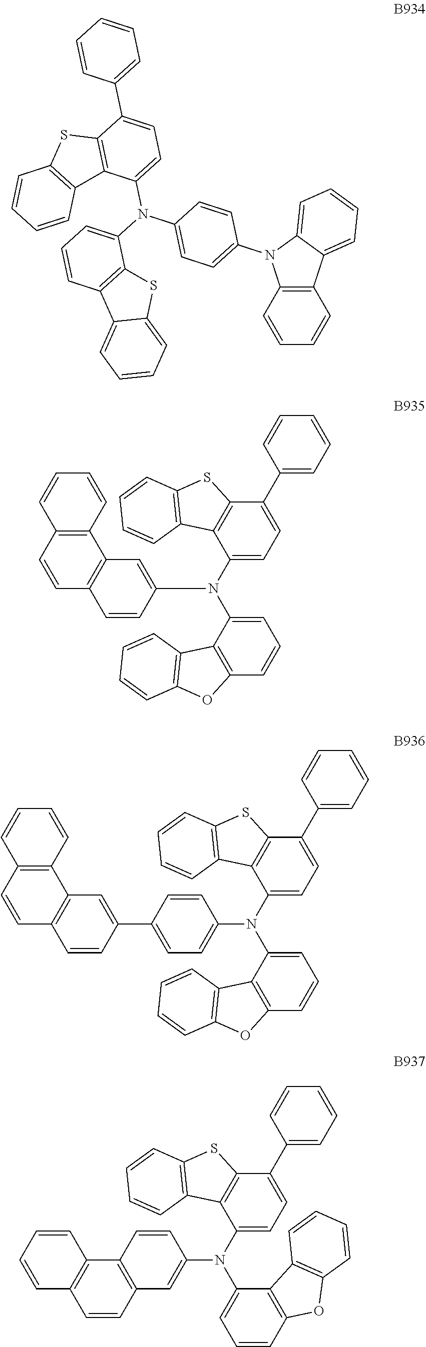 [SMILES: c1ccc(-c2ccc(N(c3ccc(-n4c5ccccc5c5ccccc54)cc3)c3cccc4c3sc3ccccc34)c3c2sc2ccccc23)cc1 ; c1ccc(-c2ccc(N(c3ccc4ccc5ccccc5c4c3)c3cccc4oc5ccccc5c34)c3c2sc2ccccc23)cc1 ; c1ccc(-c2ccc(N(c3ccc(-c4ccc5ccc6ccccc6c5c4)cc3)c3cccc4oc5ccccc5c34)c3c2sc2ccccc23)cc1 ; c1ccc(-c2ccc(N(c3ccc4c(ccc5ccccc54)c3)c3cccc4oc5ccccc5c34)c3c2sc2ccccc23)cc1]

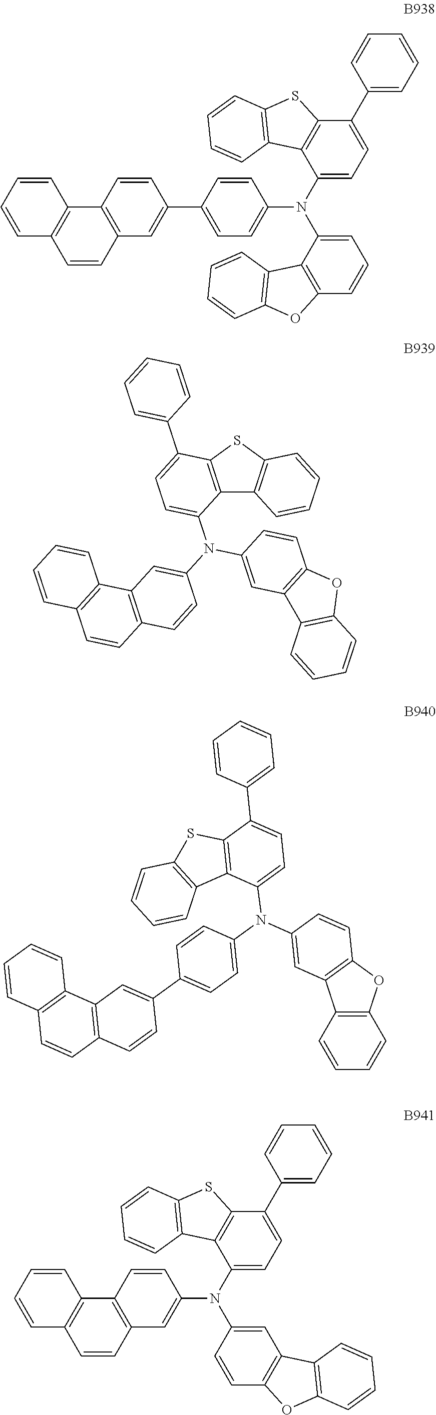 [SMILES: c1ccc(-c2ccc(N(c3ccc(-c4ccc5c(ccc6ccccc65)c4)cc3)c3cccc4oc5ccccc5c34)c3c2sc2ccccc23)cc1 ; c1ccc(-c2ccc(N(c3ccc4ccc5ccccc5c4c3)c3ccc4oc5ccccc5c4c3)c3c2sc2ccccc23)cc1 ; c1ccc(-c2ccc(N(c3ccc(-c4ccc5ccc6ccccc6c5c4)cc3)c3ccc4oc5ccccc5c4c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1]

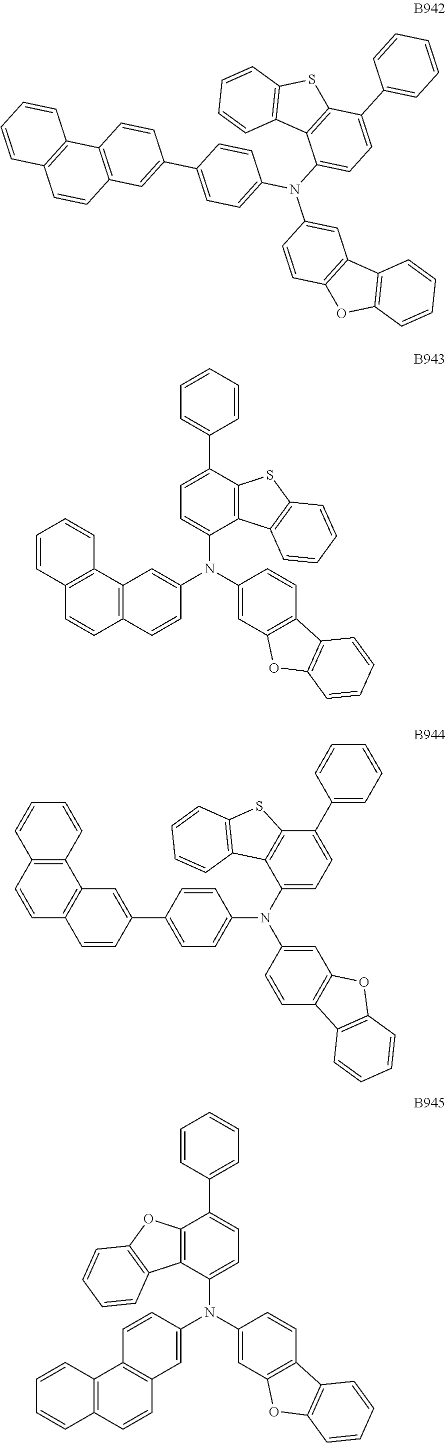

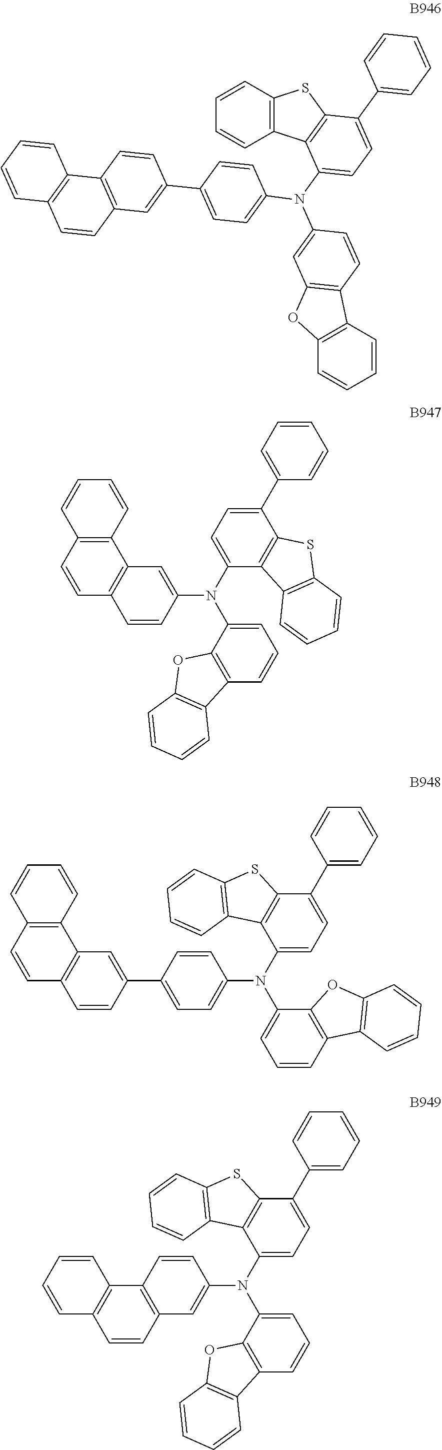

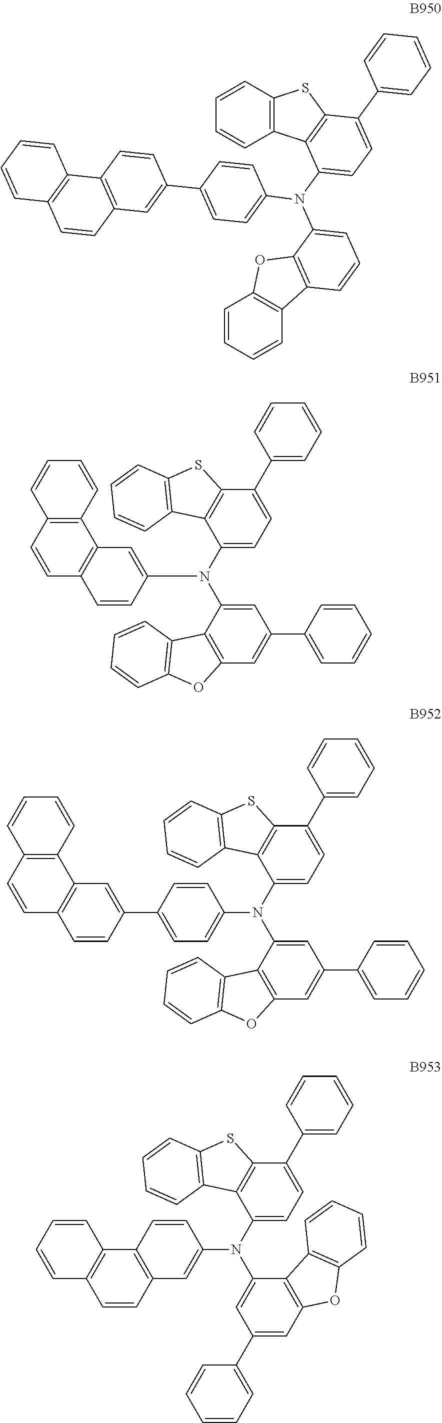 [SMILES: c1ccc(-c2ccc(N(c3ccc(-c4ccc5c(ccc6ccccc65)c4)cc3)c3cccc4c3oc3ccccc34)c3c2sc2ccccc23)cc1 ; c1ccc(-c2cc(N(c3ccc4ccc5ccccc5c4c3)c3ccc(-c4ccccc4)c4sc5ccccc5c34)c3c(c2)oc2ccccc23)cc1 ; c1ccc(-c2cc(N(c3ccc(-c4ccc5ccc6ccccc6c5c4)cc3)c3ccc(-c4ccccc4)c4sc5ccccc5c34)c3c(c2)oc2ccccc23)cc1 ; c1ccc(-c2cc(N(c3ccc4c(ccc5ccccc54)c3)c3ccc(-c4ccccc4)c4sc5ccccc5c34)c3c(c2)oc2ccccc23)cc1]

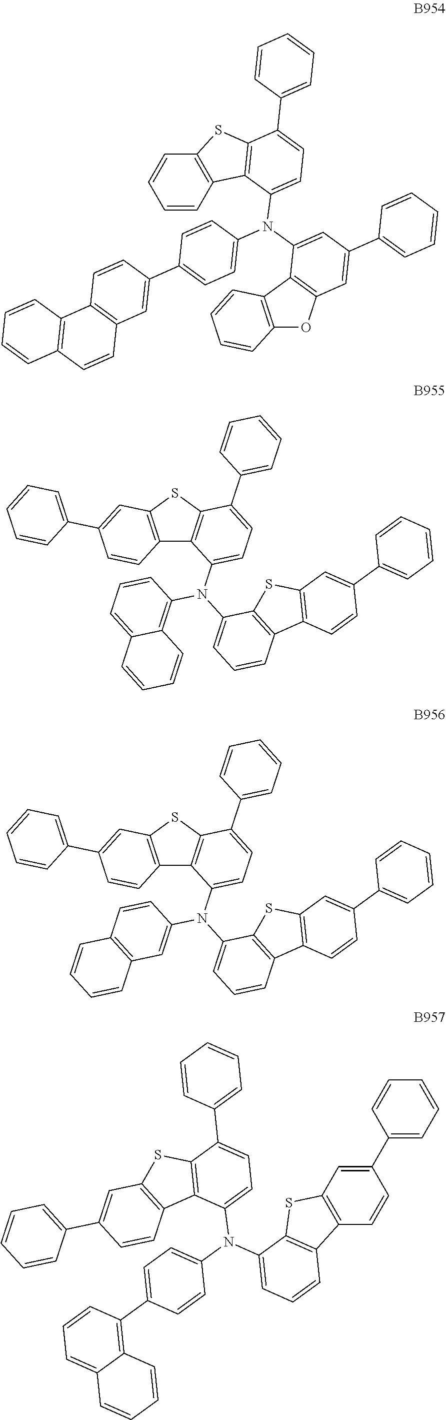 [SMILES: c1ccc(-c2cc(N(c3ccc(-c4ccc5c(ccc6ccccc65)c4)cc3)c3ccc(-c4ccccc4)c4sc5ccccc5c34)c3c(c2)oc2ccccc23)cc1 ; c1ccc(-c2ccc3c(c2)sc2c(N(c4cccc5ccccc45)c4ccc(-c5ccccc5)c5sc6cc(-c7ccccc7)ccc6c45)cccc23)cc1 ; c1ccc(-c2ccc3c(c2)sc2c(N(c4ccc5ccccc5c4)c4ccc(-c5ccccc5)c5sc6cc(-c7ccccc7)ccc6c45)cccc23)cc1 ; c1ccc(-c2ccc3c(c2)sc2c(N(c4ccc(-c5cccc6ccccc56)cc4)c4ccc(-c5ccccc5)c5sc6cc(-c7ccccc7)ccc6c45)cccc23)cc1]

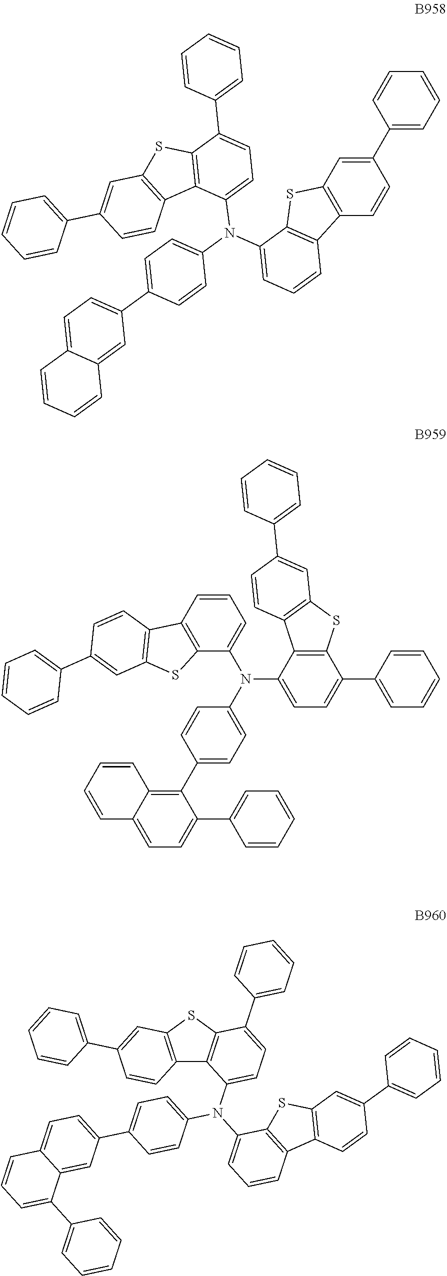 [SMILES: c1ccc(-c2ccc3c(c2)sc2c(N(c4ccc(-c5ccc6ccccc6c5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5c(-c6ccccc6)ccc6ccccc56)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6cccc(-c7ccccc7)c6c5)cc4)c4ccc(-c5ccccc5)c5sc6cc(-c7ccccc7)ccc6c45)cccc23)cc1]

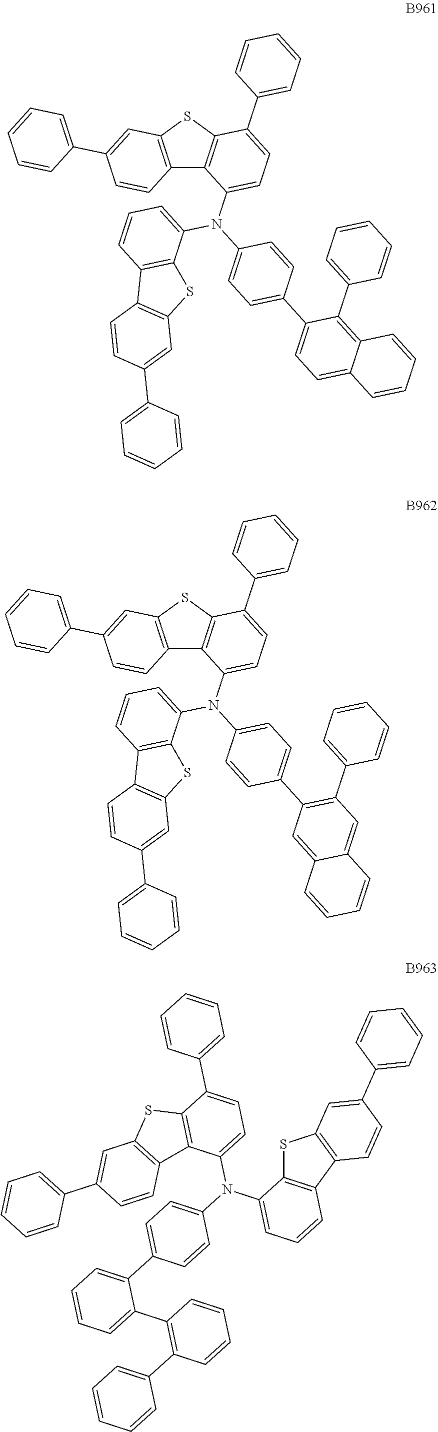 [SMILES: c1ccc(-c2ccc3c(c2)sc2c(N(c4ccc(-c5ccc6ccccc6c5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5cc6ccccc6cc5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccccc5-c5ccccc5-c5ccccc5)cc4)c4ccc(-c5ccccc5)c5sc6cc(-c7ccccc7)ccc6c45)cccc23)cc1]

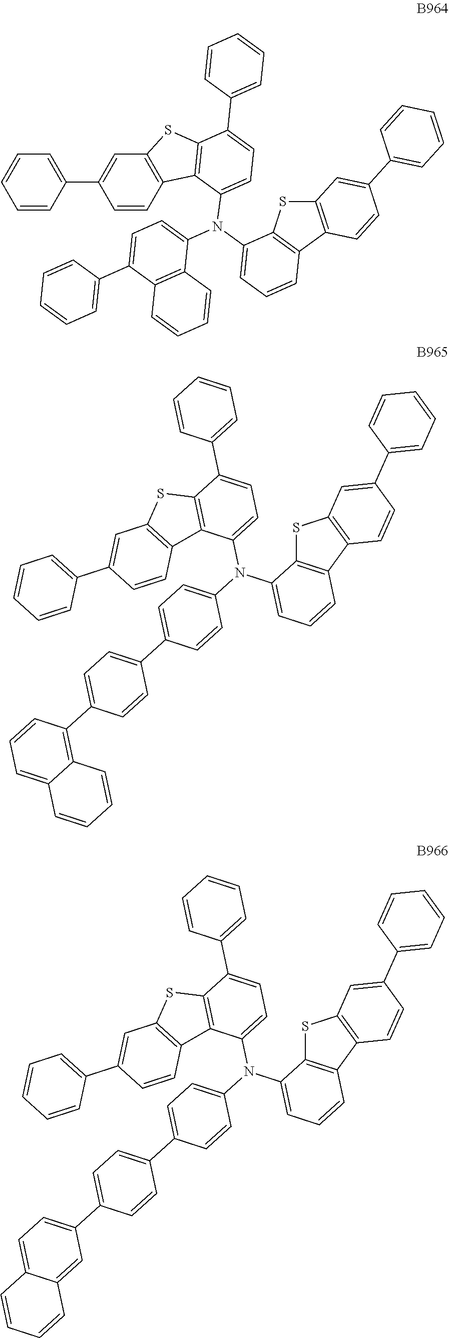

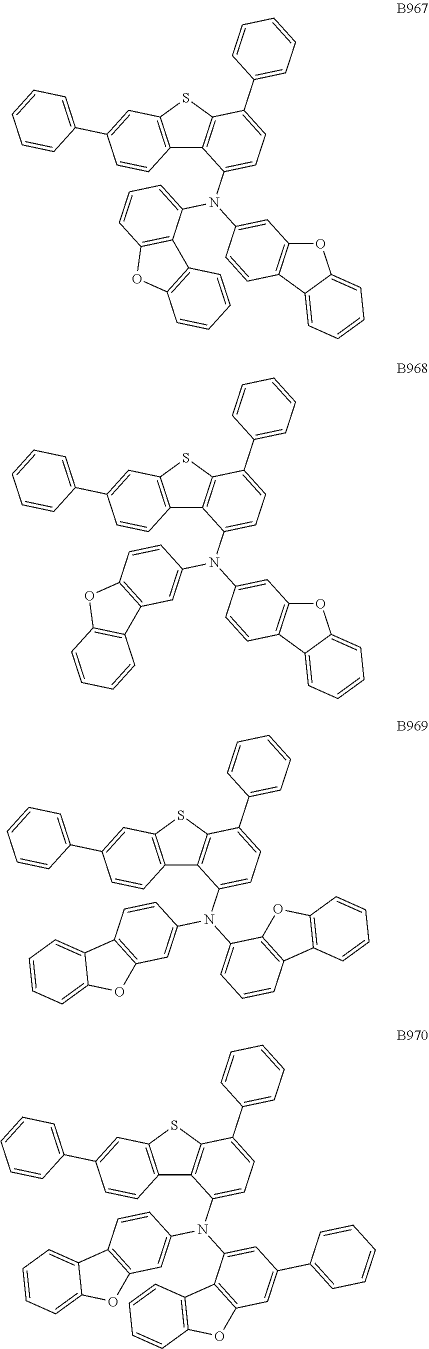 [SMILES: c1ccc(-c2ccc3c(c2)sc2c(-c4ccccc4)ccc(N(c4ccc5c(c4)oc4ccccc45)c4cccc5oc6ccccc6c45)c23)cc1 ; c1ccc(-c2ccc3c(c2)sc2c(-c4ccccc4)ccc(N(c4ccc5c(c4)oc4ccccc45)c4ccc5oc6ccccc6c5c4)c23)cc1 ; c1ccc(-c2ccc3c(c2)sc2c(-c4ccccc4)ccc(N(c4ccc5c(c4)oc4ccccc45)c4cccc5c4oc4ccccc45)c23)cc1 ; c1ccc(-c2cc(N(c3ccc4c(c3)oc3ccccc34)c3ccc(-c4ccccc4)c4sc5cc(-c6ccccc6)ccc5c34)c3c(c2)oc2ccccc23)cc1]

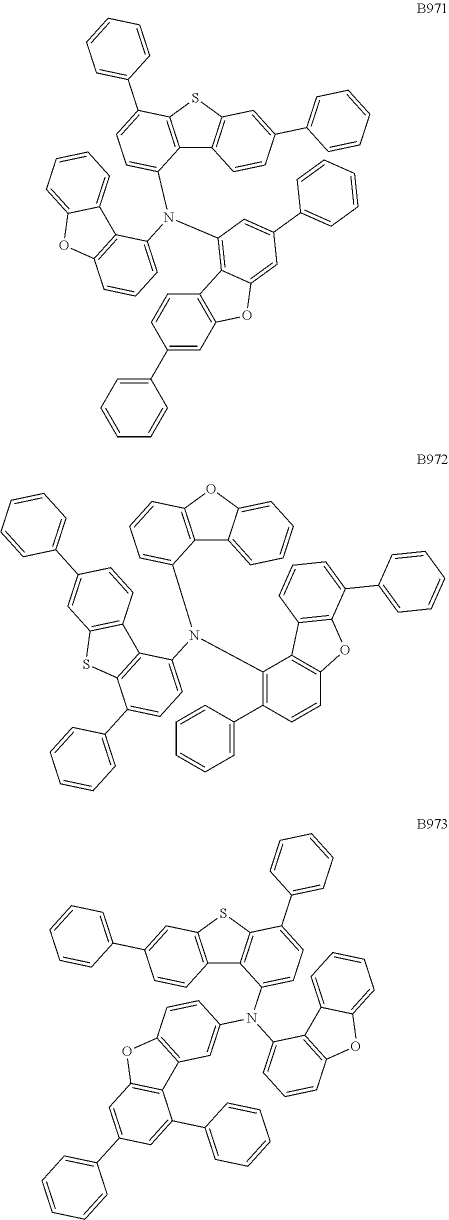 [SMILES: c1ccc(-c2ccc3c(c2)oc2cc(-c4ccccc4)cc(N(c4cccc5oc6ccccc6c45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5oc6ccccc6c45)c4c(-c5ccccc5)ccc5oc6c(-c7ccccc7)cccc6c45)c23)cc1 ; c1ccc(-c2cc(-c3ccccc3)c3c(c2)oc2ccc(N(c4cccc5oc6ccccc6c45)c4ccc(-c5ccccc5)c5sc6cc(-c7ccccc7)ccc6c45)cc23)cc1]

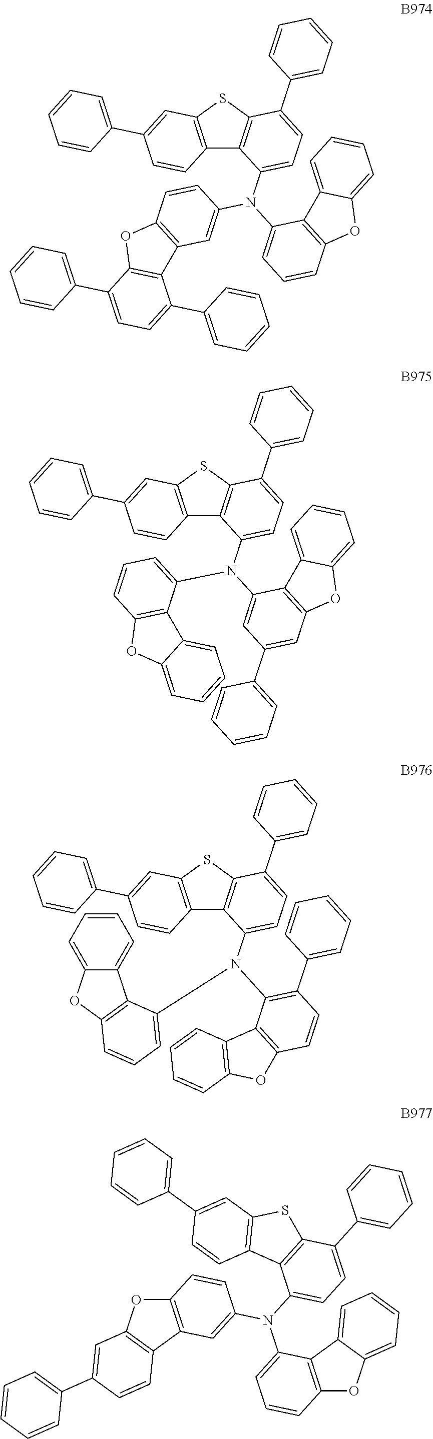

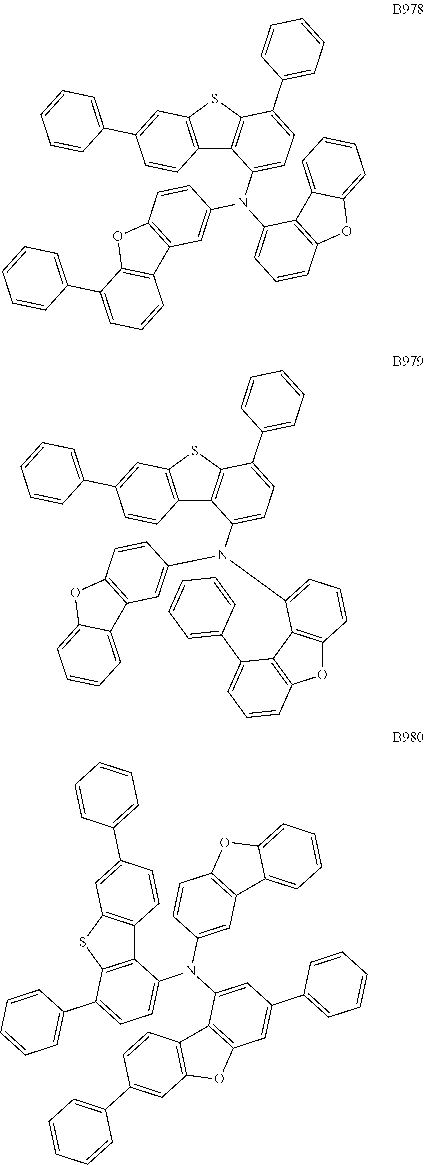 [SMILES: c1ccc(-c2ccc3c(c2)sc2c(-c4ccccc4)ccc(N(c4ccc5oc6c(-c7ccccc7)cccc6c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5oc6ccccc6c5c4)c4ccc(-c5ccccc5)c5sc6cc(-c7ccccc7)ccc6c45)c23)cc1]

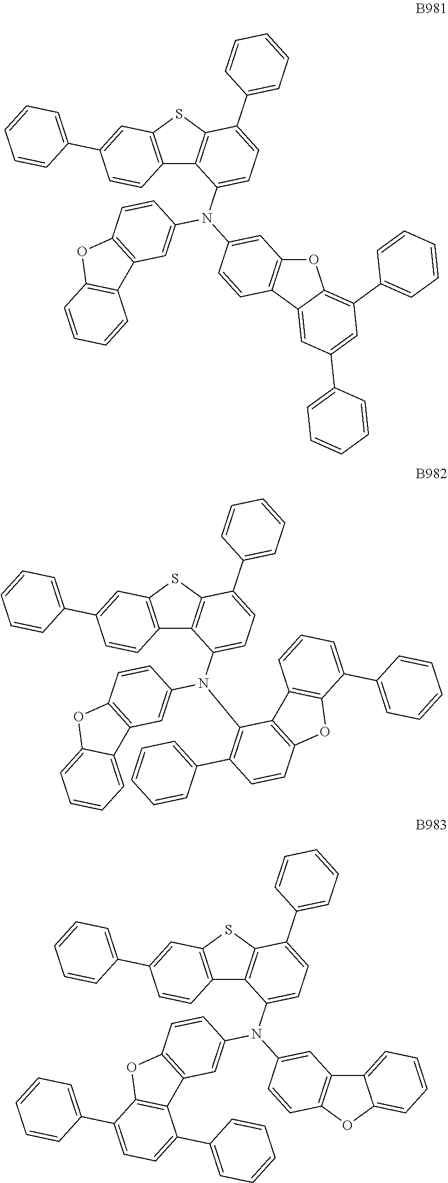 [SMILES: c1ccc(-c2ccc3c(c2)sc2c(-c4ccccc4)ccc(N(c4ccc5c(c4)oc4c(-c6ccccc6)cc(-c6ccccc6)cc45)c4ccc5oc6ccccc6c5c4)c23)cc1 ; c1ccc(-c2ccc3c(c2)sc2c(-c4ccccc4)ccc(N(c4ccc5oc6ccccc6c5c4)c4c(-c5ccccc5)ccc5oc6c(-c7ccccc7)cccc6c45)c23)cc1 ; c1ccc(-c2ccc3c(c2)sc2c(-c4ccccc4)ccc(N(c4ccc5oc6ccccc6c5c4)c4ccc5oc6c(-c7ccccc7)ccc(-c7ccccc7)c6c5c4)c23)cc1]

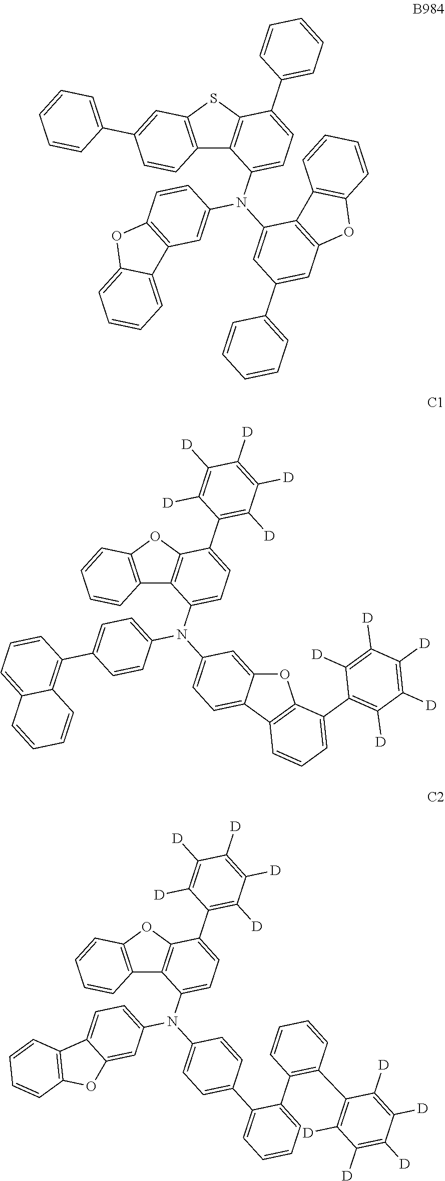 [SMILES: c1ccc(-c2cc(N(c3ccc4oc5ccccc5c4c3)c3ccc(-c4ccccc4)c4sc5cc(-c6ccccc6)ccc5c34)c3c(c2)oc2ccccc23)cc1 ; [2H]c1c([2H])c([2H])c(-c2cccc3c2oc2cc(N(c4ccc(-c5cccc6ccccc56)cc4)c4ccc(-c5c([2H])c([2H])c([2H])c([2H])c5[2H])c5oc6ccccc6c45)ccc23)c([2H])c1[2H] ; [2H]c1c([2H])c([2H])c(-c2ccccc2-c2ccccc2-c2ccc(N(c3ccc4c(c3)oc3ccccc34)c3ccc(-c4c([2H])c([2H])c([2H])c([2H])c4[2H])c4oc5ccccc5c34)cc2)c([2H])c1[2H]]

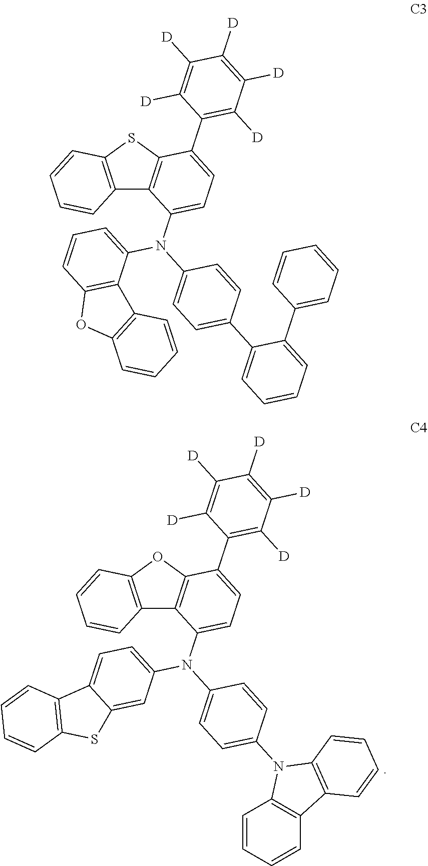

15. An amine compound represented by Formula 1: 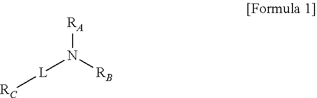
in Formula 1,
R_{A} is represented by Formula 2,
R_{B} is represented by Formula 3,
R_{C} is a substituted or unsubstituted aryl group of 6 to 30 ring-forming carbon atoms, or a substituted or unsubstituted heteroaryl group of 2 to 30 ring-forming carbon atoms,
L is a direct linkage, or a substituted or unsubstituted arylene group of 6 to 30 ring-forming carbon atoms,
R_{C} is not a substituted or unsubstituted benzonaphthofuran group, a substituted or unsubstituted benzonaphthothiophene group, or a substituted or unsubstituted benzofluorene group,
when R_{C} is a substituted or unsubstituted dibenzofuran group, or a substituted or unsubstituted dibenzothiophene group, L is a direct linkage, and
when R_{C} is a substituted or unsubstituted carbazole group, L is a direct linkage, or a substituted or unsubstituted phenylene group: 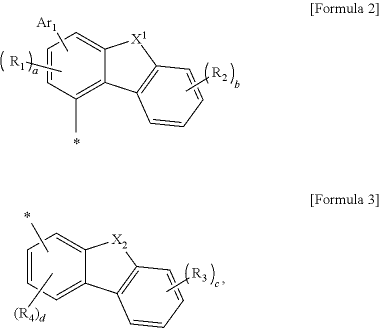
and
in Formula 2 and Formula 3,
Ar₁ is a substituted or unsubstituted aryl group of 6 to 30 ring-forming carbon atoms,
X₁ and X₂ are each independently O or S,
R₁ to R₄ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
wherein,
R₁ to R₄ are not each comprise a substituted or unsubstituted nitrogen-containing six-member heterocycle,
Ar₁ is not a substituted or unsubstituted dihydronaphthyl group, and a substituted or unsubstituted p-aminophenyl group,
R₃ is not a substituted or unsubstituted fluorene group,
a is an integer of 0 to 2,
d is an integer of 0 to 3,
b and c are each independently an integer of 0 to 4, and
in Formula 2 when X₁ is O, Formula 3 is not represented by Formula 3-a:

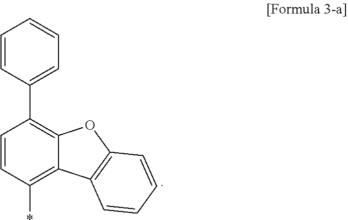 [SMILES: *c1ccc(-c2ccccc2)c2oc3ccccc3c12]

16. The amine compound of claim 15, wherein the amine compound represented by Formula 1 is represented by Formula 1-1: 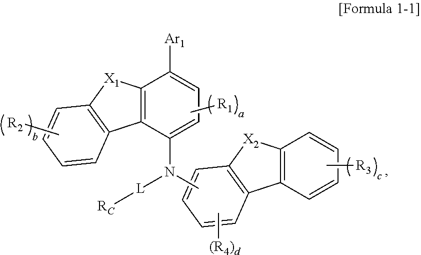
and
in Formula 1-1,
R_{C}, L, Ar₁, X₁, X₂, R₁ to R₄, and a to d are as defined in Formula 1, Formula 2 and Formula 3.

17. The amine compound of claim 15, wherein the amine compound represented by Formula 1 is represented by Formula 1-2-1 or Formula 1-2-2: 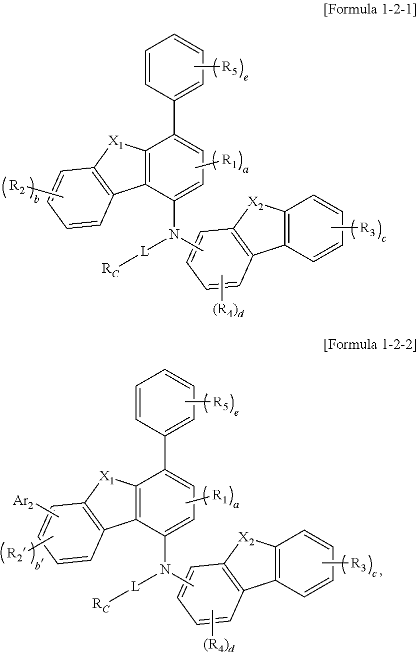
and
in Formula 1-2-1 and Formula 1-2-2,
R₂′ and R₅ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Ar₂ is a substituted or unsubstituted aryl group of 6 to 30 ring-forming carbon atoms, or a substituted or unsubstituted heteroaryl group of 2 to 30 ring-forming carbon atoms,
b′ is an integer of 0 to 3,
e is an integer of 0 to 5, and
R_{C}, L, X₁, X₂, R₁ to R₄, and a to d are as defined in Formula 1, Formula 2, and Formula 3.

18. The amine compound of claim 15, wherein the substituent represented by Formula R_{C} is represented by one of Formula A-1 to Formula A-8: 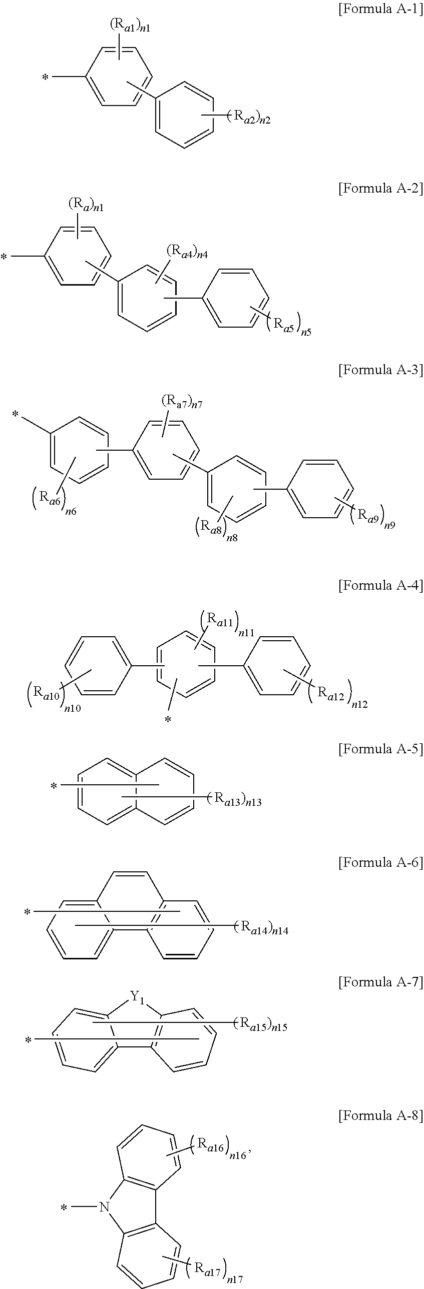
and
in Formula A-1 to Formula A-7,
Y₁ is O, S, CRₐ₁₈Rₐ₁₉, or NRₐ₂₀,
Rₐ₁ to Rₐ₁₉ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
Rₐ₂₀ is a substituted or unsubstituted aryl group of 6 to 30 ring-forming carbon atoms, or a substituted or unsubstituted heteroaryl group of 2 to 30 ring-forming carbon atoms,
n1, n3, n4, and n6 to n8 are each independently an integer of 0 to 4,
n2, n5, n9, n10, and n12 are each independently an integer of 0 to 5,
n11 is an integer of 0 to 3,
n13 and n15 are each independently an integer of 0 to 7,
n14 is an integer of 0 to 9,
n16 and n17 are each independently an integer of 0 to 4, and
-* is a position connected with L of Formula 1.

19. The amine compound of claim 15, wherein the amine compound represented by Formula 1 is represented by one of Formula 1-4-1 to Formula 1-4-3: 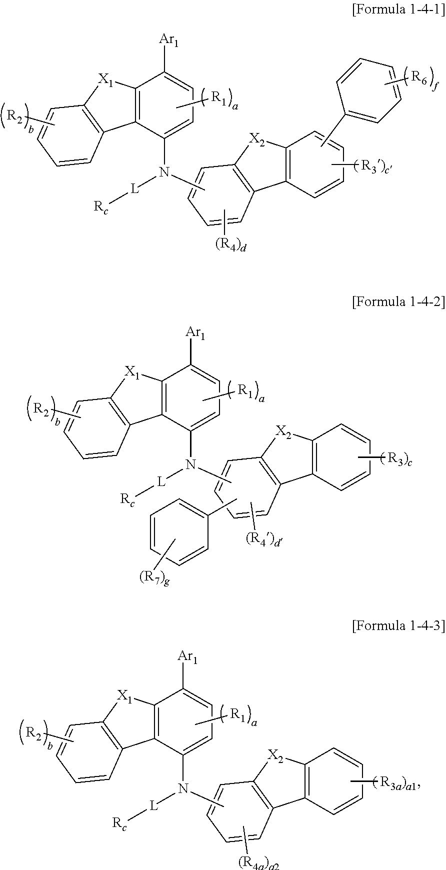
and
in Formula 1-4-1 to Formula 1-4-3,
R₃′, R₄′, R₆ and R₇ are each independently a hydrogen atom, a deuterium atom a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
R₃ₐ and R₄ₐ are each independently a hydrogen atom, or a deuterium atom,
f and g are each independently an integer of 0 to 5,
c′ and a2 are each independently an integer of 0 to 3,
d′ is an integer of 0 to 2,
a1 is an integer of 0 to 4, and
R_{C}, L, Ar₁, X₁, X₂, R₁ to R₄, and a to d are as defined in Formula 1, Formula 2, and Formula 3.

20. The amine compound of claim 15, wherein the amine compound represented by Formula 1 is represented by one of Formula 1-5-1 or Formula 1-5-2: 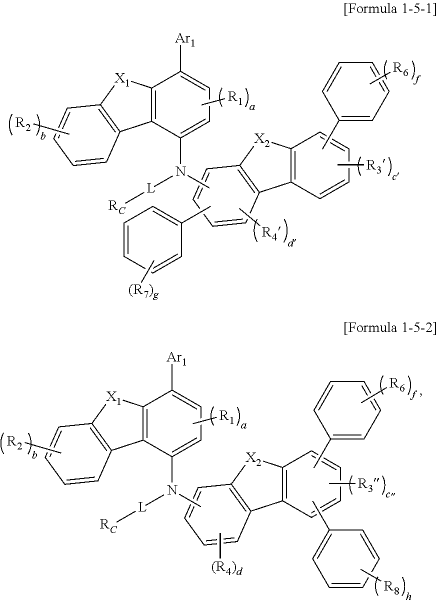
and
in Formula 1-5-1 and Formula 1-5-2,
R₃′, R₃″, R₄′, and R₆ to R₈ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
f to h are each independently an integer of 0 to 5,
c′ is an integer of 0 to 3,
c″ and d′ are each independently an integer of 0 to 2, and
R_{C}, L, Ar₁, X₁, X₂, R₁ R₂, R₄, a, b, and d are as defined in Formula 1, Formula 2, and Formula 3.

21. The amine compound of claim 15, wherein the amine compound represented by Formula 1 is represented by one of Formula 1-6-1 to Formula 1-6-4: 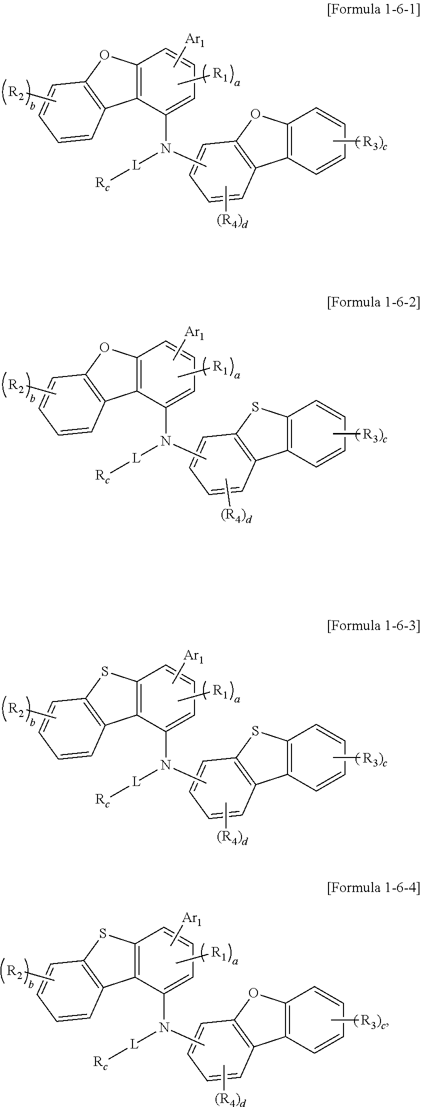
and
in Formula 1-6-1 to Formula 1-6-4,
R_{C}, L, Ar₁, R₁ to R₄, and a to d are as defined in Formula 1, Formula 2, and Formula 3.

22. The amine compound of claim 15, wherein the amine compound represented by Formula 1 is selected from Compound Group 1:

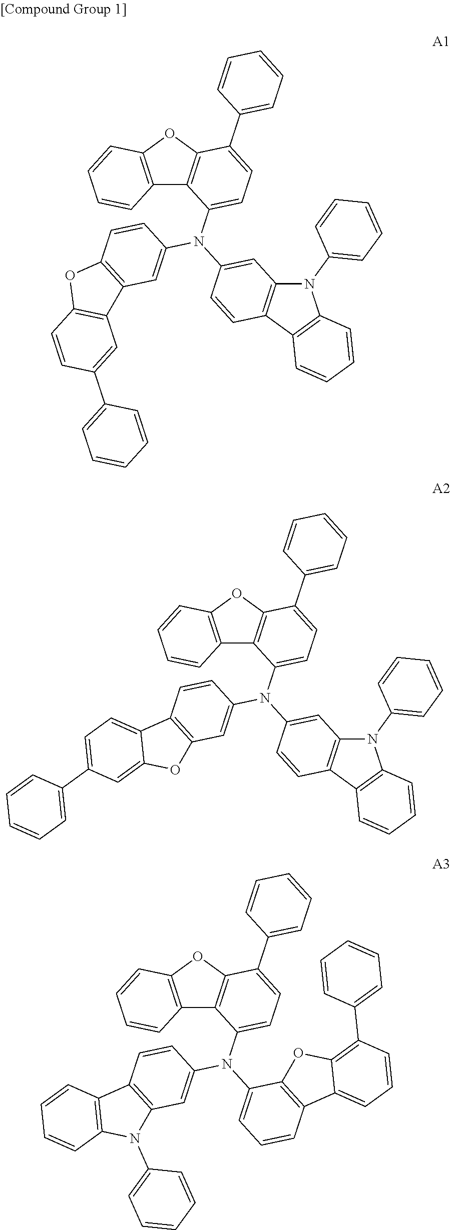 [SMILES: c1ccc(-c2ccc3oc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)oc2cc(N(c4ccc5c6ccccc6n(-c6ccccc6)c5c4)c4ccc(-c5ccccc5)c5oc6ccccc6c45)ccc23)cc1 ; c1ccc(-c2cccc3c2oc2c(N(c4ccc5c6ccccc6n(-c6ccccc6)c5c4)c4ccc(-c5ccccc5)c5oc6ccccc6c45)cccc23)cc1]

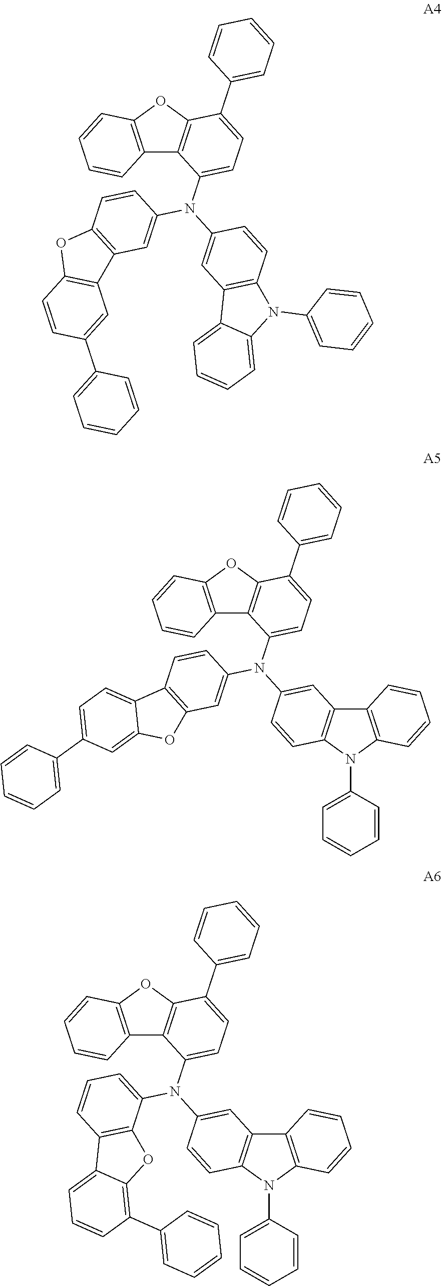 [SMILES: c1ccc(-c2ccc3oc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)oc2cc(N(c4ccc5c(c4)c4ccccc4n5-c4ccccc4)c4ccc(-c5ccccc5)c5oc6ccccc6c45)ccc23)cc1 ; c1ccc(-c2cccc3c2oc2c(N(c4ccc5c(c4)c4ccccc4n5-c4ccccc4)c4ccc(-c5ccccc5)c5oc6ccccc6c45)cccc23)cc1]

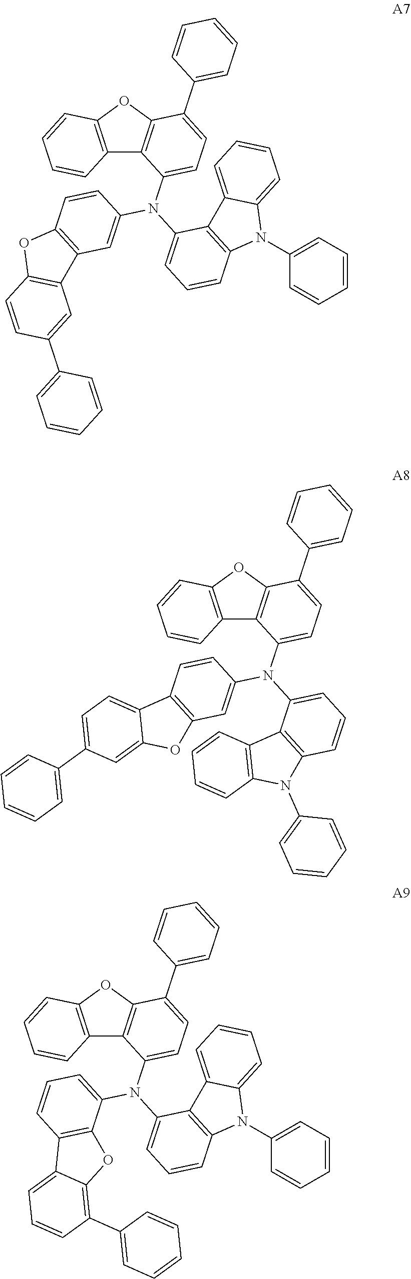 [SMILES: c1ccc(-c2ccc3oc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)oc2cc(N(c4ccc(-c5ccccc5)c5oc6ccccc6c45)c4cccc5c4c4ccccc4n5-c4ccccc4)ccc23)cc1 ; c1ccc(-c2cccc3c2oc2c(N(c4ccc(-c5ccccc5)c5oc6ccccc6c45)c4cccc5c4c4ccccc4n5-c4ccccc4)cccc23)cc1]

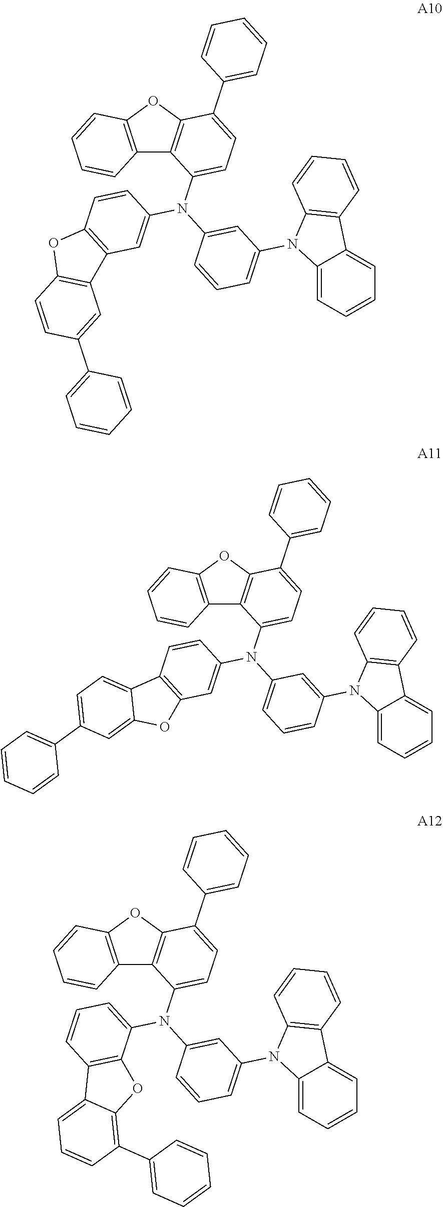 [SMILES: c1ccc(-c2ccc3oc4ccc(N(c5cccc(-n6c7ccccc7c7ccccc76)c5)c5ccc(-c6ccccc6)c6oc7ccccc7c56)cc4c3c2)cc1 ; c1ccc(-c2ccc3c(c2)oc2cc(N(c4cccc(-n5c6ccccc6c6ccccc65)c4)c4ccc(-c5ccccc5)c5oc6ccccc6c45)ccc23)cc1 ; c1ccc(-c2cccc3c2oc2c(N(c4cccc(-n5c6ccccc6c6ccccc65)c4)c4ccc(-c5ccccc5)c5oc6ccccc6c45)cccc23)cc1]

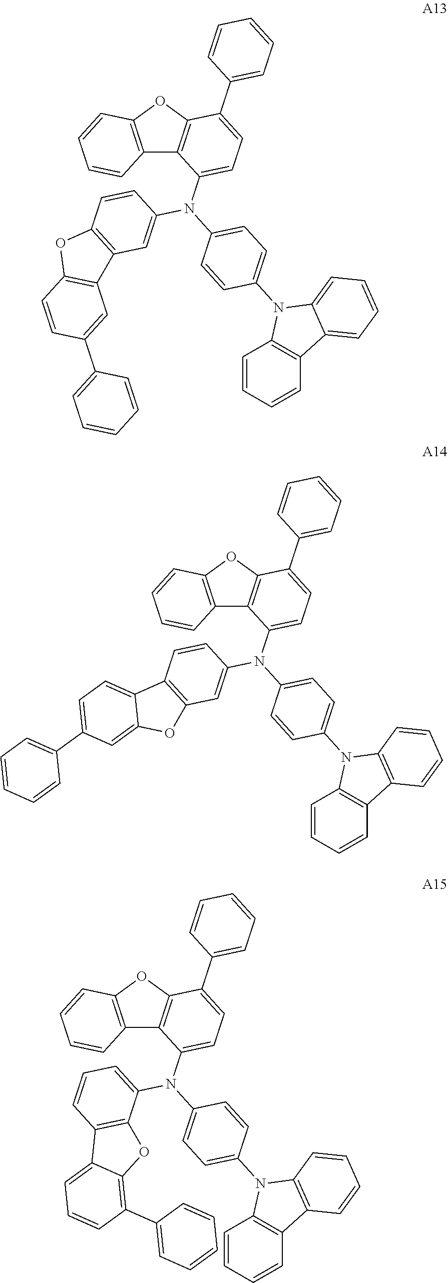 [SMILES: c1ccc(-c2ccc3oc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)oc2cc(N(c4ccc(-n5c6ccccc6c6ccccc65)cc4)c4ccc(-c5ccccc5)c5oc6ccccc6c45)ccc23)cc1 ; c1ccc(-c2cccc3c2oc2c(N(c4ccc(-n5c6ccccc6c6ccccc65)cc4)c4ccc(-c5ccccc5)c5oc6ccccc6c45)cccc23)cc1]

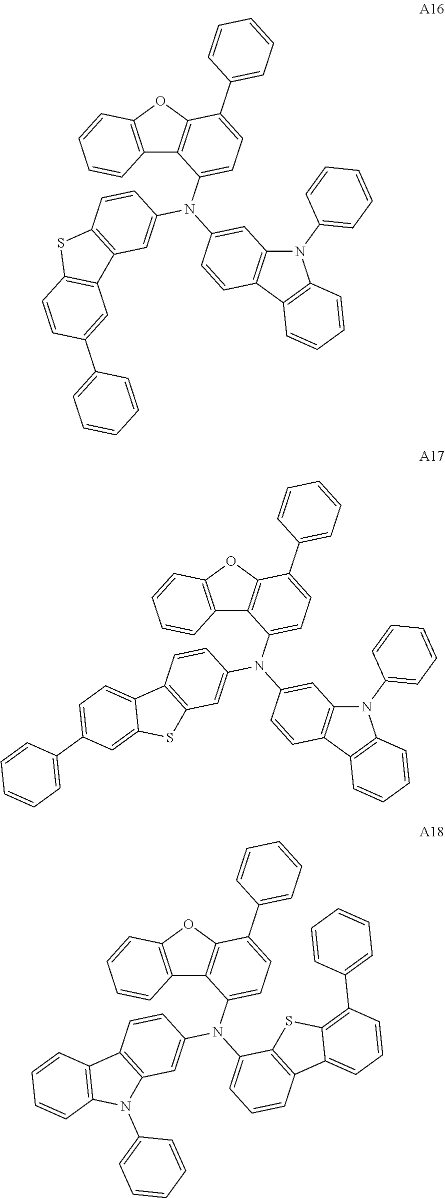 [SMILES: c1ccc(-c2ccc3sc4ccc(N(c5ccc6c7ccccc7n(-c7ccccc7)c6c5)c5ccc(-c6ccccc6)c6oc7ccccc7c56)cc4c3c2)cc1 ; c1ccc(-c2ccc3c(c2)sc2cc(N(c4ccc5c6ccccc6n(-c6ccccc6)c5c4)c4ccc(-c5ccccc5)c5oc6ccccc6c45)ccc23)cc1 ; c1ccc(-c2ccc(N(c3ccc4c5ccccc5n(-c5ccccc5)c4c3)c3cccc4c3sc3c(-c5ccccc5)cccc34)c3c2oc2ccccc23)cc1]

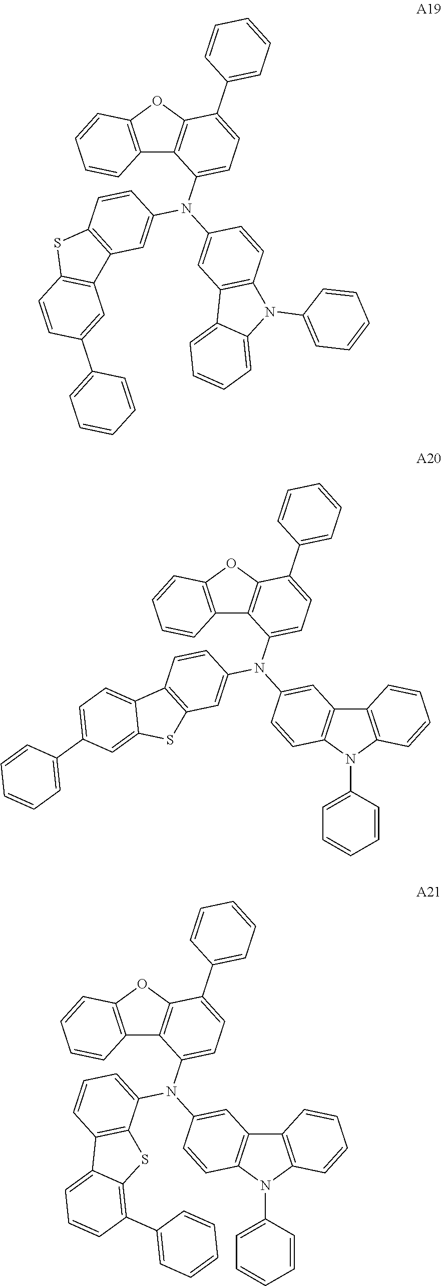 [SMILES: c1ccc(-c2ccc3sc4ccc(N(c5ccc6c(c5)c5ccccc5n6-c5ccccc5)c5ccc(-c6ccccc6)c6oc7ccccc7c56)cc4c3c2)cc1 ; c1ccc(-c2ccc3c(c2)sc2cc(N(c4ccc5c(c4)c4ccccc4n5-c4ccccc4)c4ccc(-c5ccccc5)c5oc6ccccc6c45)ccc23)cc1 ; c1ccc(-c2ccc(N(c3ccc4c(c3)c3ccccc3n4-c3ccccc3)c3cccc4c3sc3c(-c5ccccc5)cccc34)c3c2oc2ccccc23)cc1]

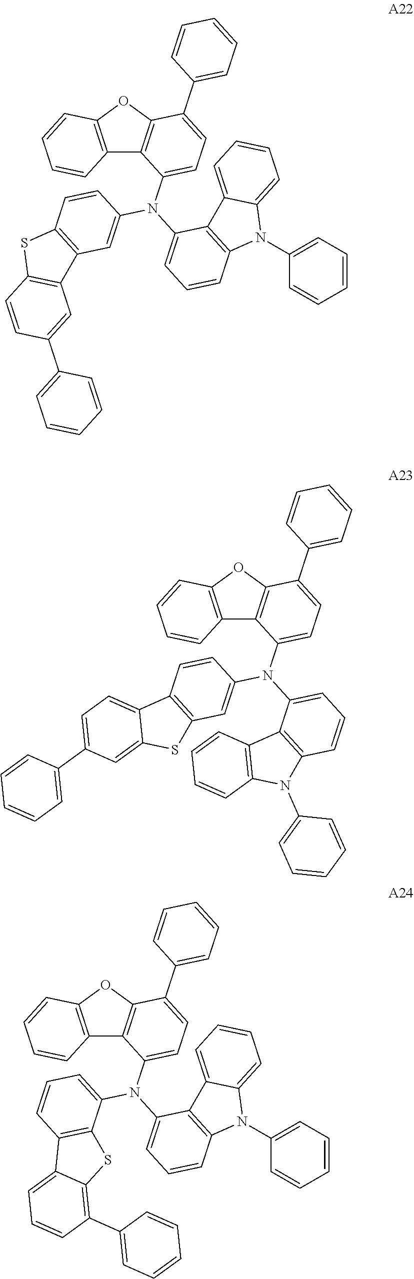 [SMILES: c1ccc(-c2ccc3sc4ccc(N(c5ccc(-c6ccccc6)c6oc7ccccc7c56)c5cccc6c5c5ccccc5n6-c5ccccc5)cc4c3c2)cc1 ; c1ccc(-c2ccc3c(c2)sc2cc(N(c4ccc(-c5ccccc5)c5oc6ccccc6c45)c4cccc5c4c4ccccc4n5-c4ccccc4)ccc23)cc1 ; c1ccc(-c2ccc(N(c3cccc4c3sc3c(-c5ccccc5)cccc34)c3cccc4c3c3ccccc3n4-c3ccccc3)c3c2oc2ccccc23)cc1]

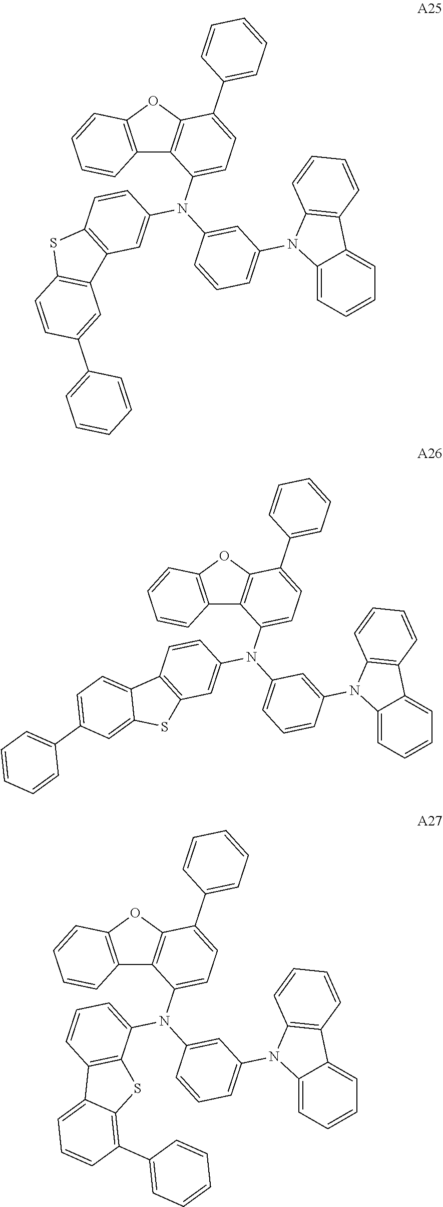 [SMILES: c1ccc(-c2ccc3sc4ccc(N(c5cccc(-n6c7ccccc7c7ccccc76)c5)c5ccc(-c6ccccc6)c6oc7ccccc7c56)cc4c3c2)cc1 ; c1ccc(-c2ccc3c(c2)sc2cc(N(c4cccc(-n5c6ccccc6c6ccccc65)c4)c4ccc(-c5ccccc5)c5oc6ccccc6c45)ccc23)cc1 ; c1ccc(-c2ccc(N(c3cccc(-n4c5ccccc5c5ccccc54)c3)c3cccc4c3sc3c(-c5ccccc5)cccc34)c3c2oc2ccccc23)cc1]

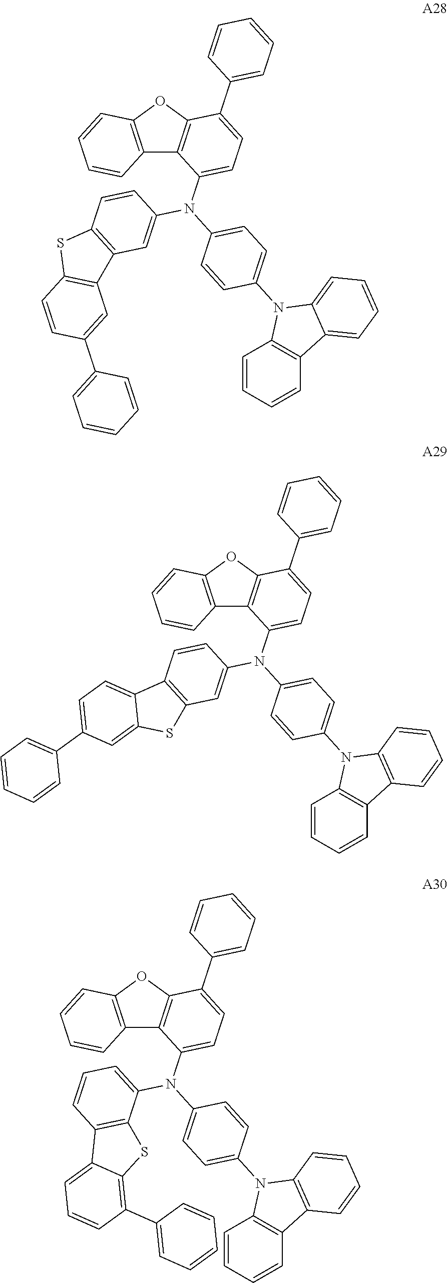 [SMILES: c1ccc(-c2ccc3sc4ccc(N(c5ccc(-n6c7ccccc7c7ccccc76)cc5)c5ccc(-c6ccccc6)c6oc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5oc6ccccc6c45)ccc23)cc1 ; c1ccc(-c2ccc(N(c3ccc(-n4c5ccccc5c5ccccc54)cc3)c3cccc4c3sc3c(-c5ccccc5)cccc34)c3c2oc2ccccc23)cc1]

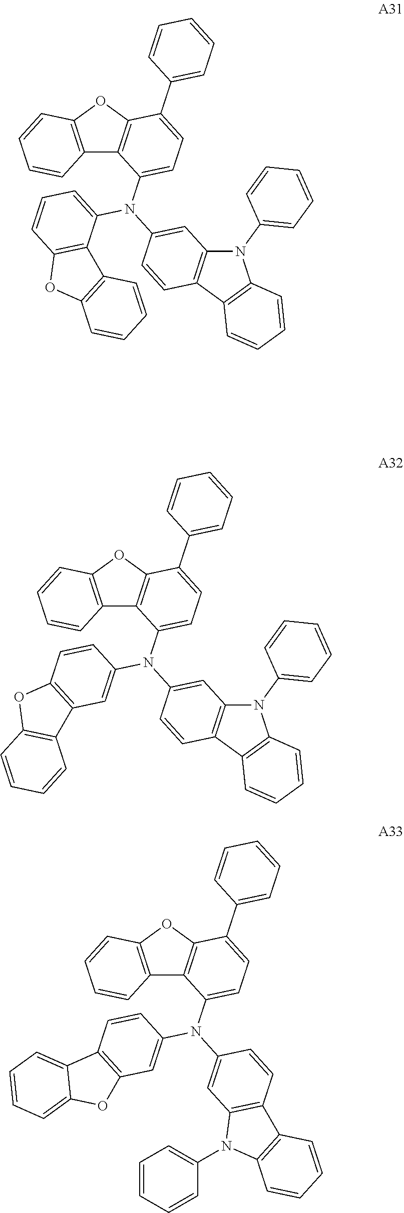

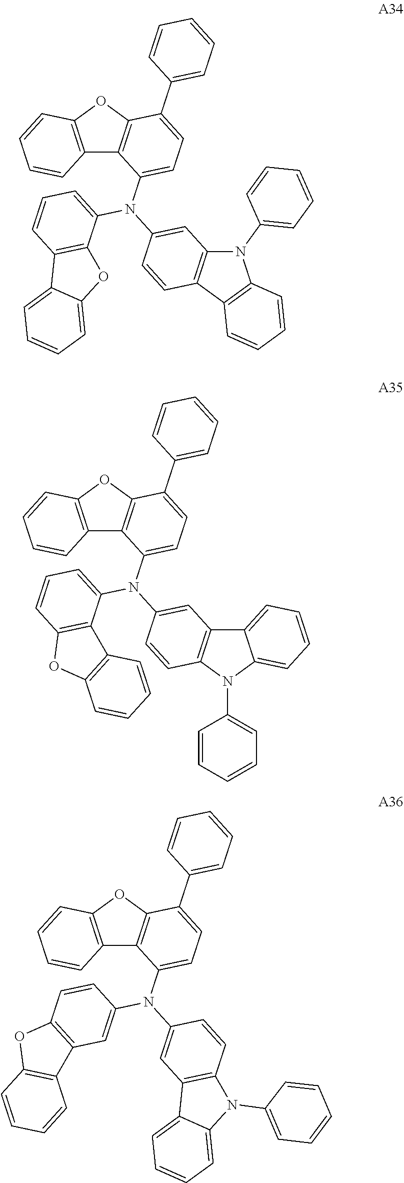

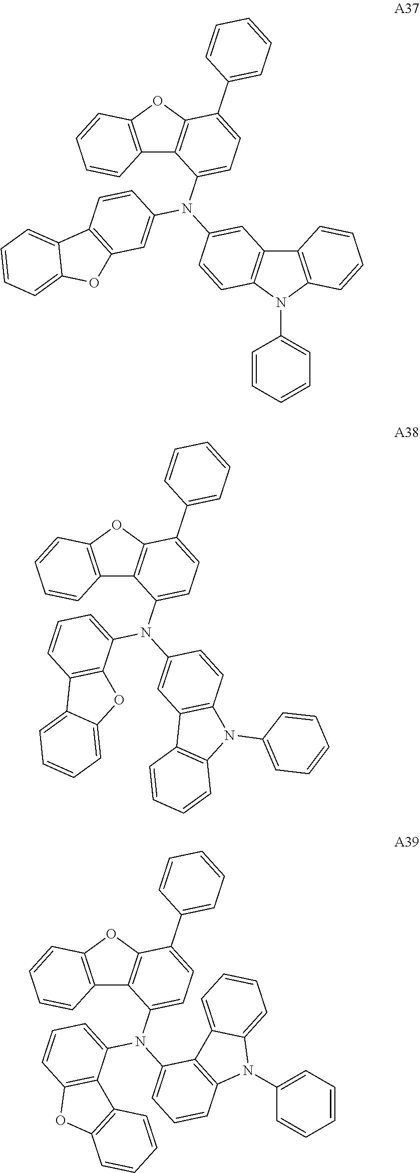

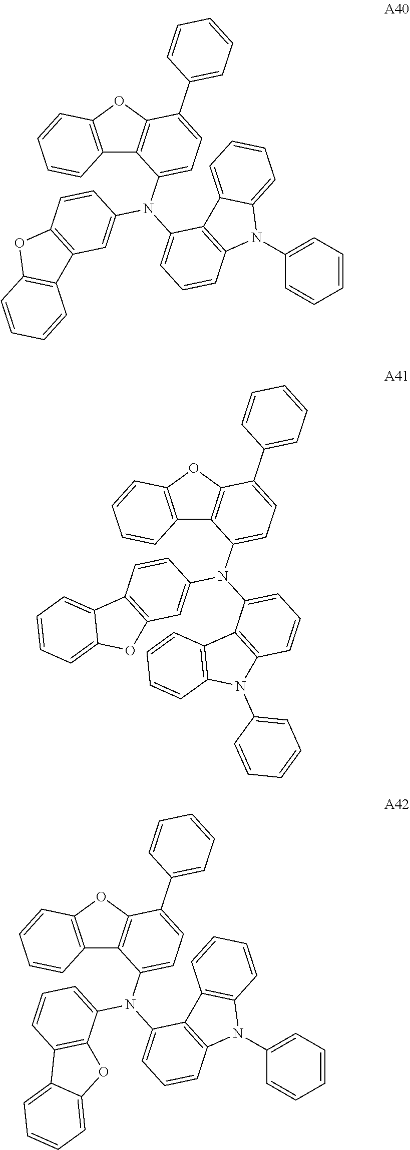 [SMILES: c1ccc(-c2ccc(N(c3ccc4oc5ccccc5c4c3)c3cccc4c3c3ccccc3n4-c3ccccc3)c3c2oc2ccccc23)cc1 ; c1ccc(-c2ccc(N(c3ccc4c(c3)oc3ccccc34)c3cccc4c3c3ccccc3n4-c3ccccc3)c3c2oc2ccccc23)cc1 ; c1ccc(-c2ccc(N(c3cccc4c3oc3ccccc34)c3cccc4c3c3ccccc3n4-c3ccccc3)c3c2oc2ccccc23)cc1]

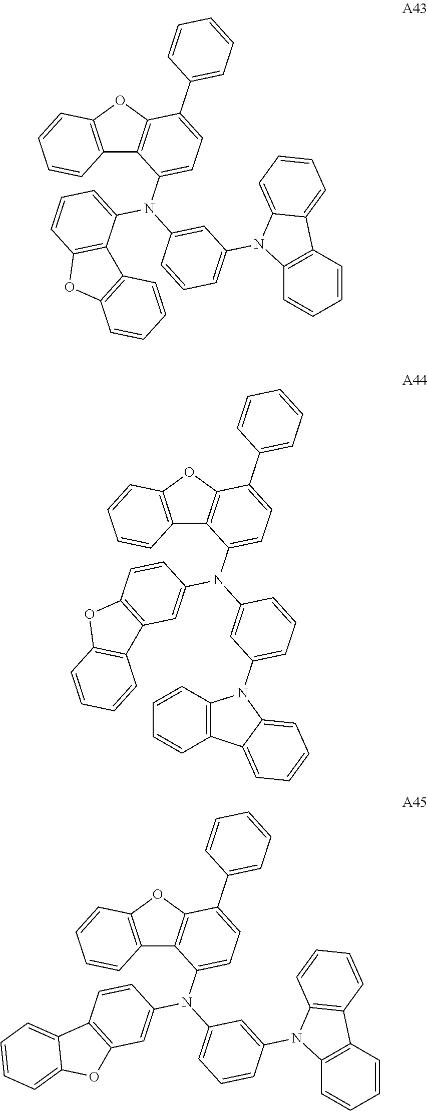

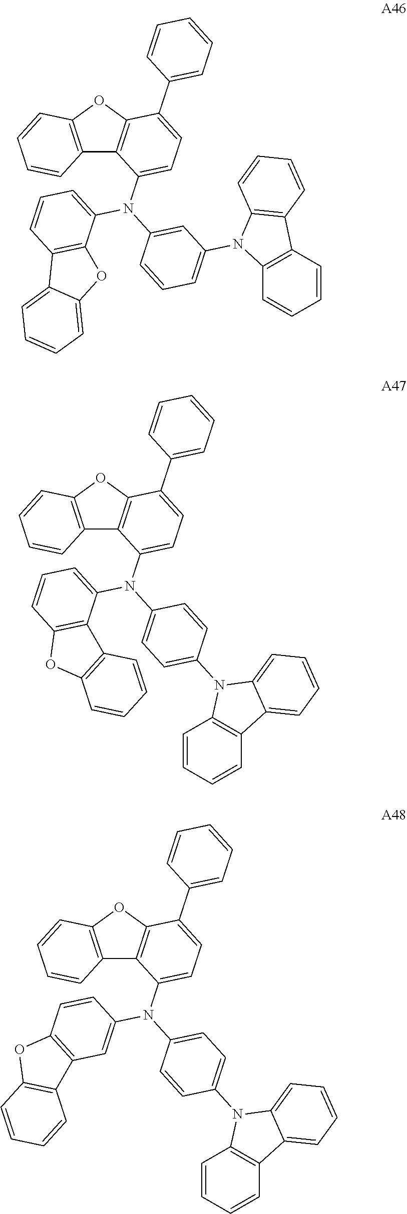

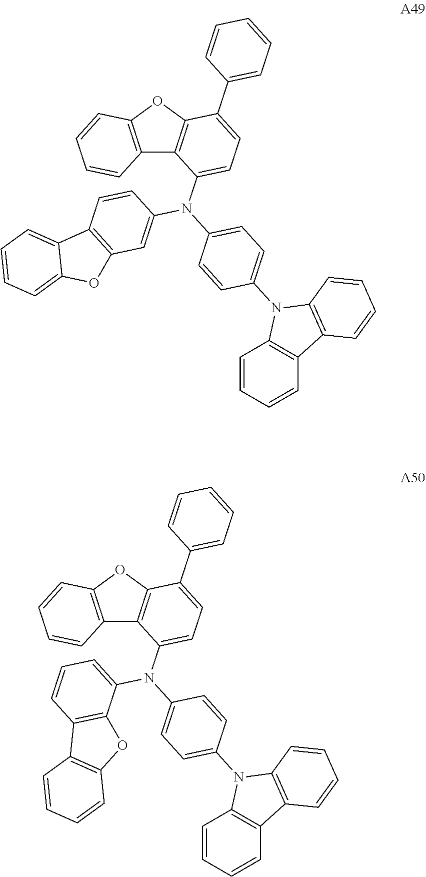 [SMILES: c1ccc(-c2ccc(N(c3ccc(-n4c5ccccc5c5ccccc54)cc3)c3ccc4c(c3)oc3ccccc34)c3c2oc2ccccc23)cc1 ; c1ccc(-c2ccc(N(c3ccc(-n4c5ccccc5c5ccccc54)cc3)c3cccc4c3oc3ccccc34)c3c2oc2ccccc23)cc1]

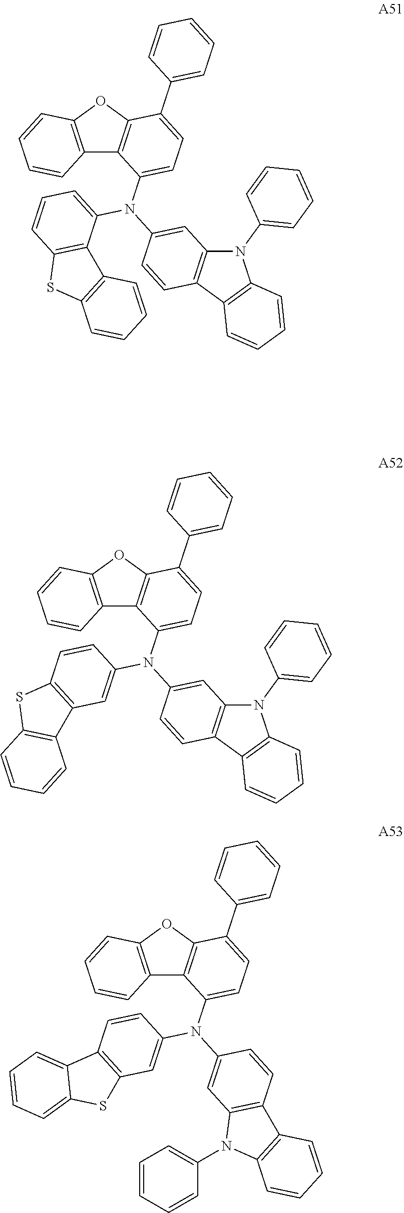

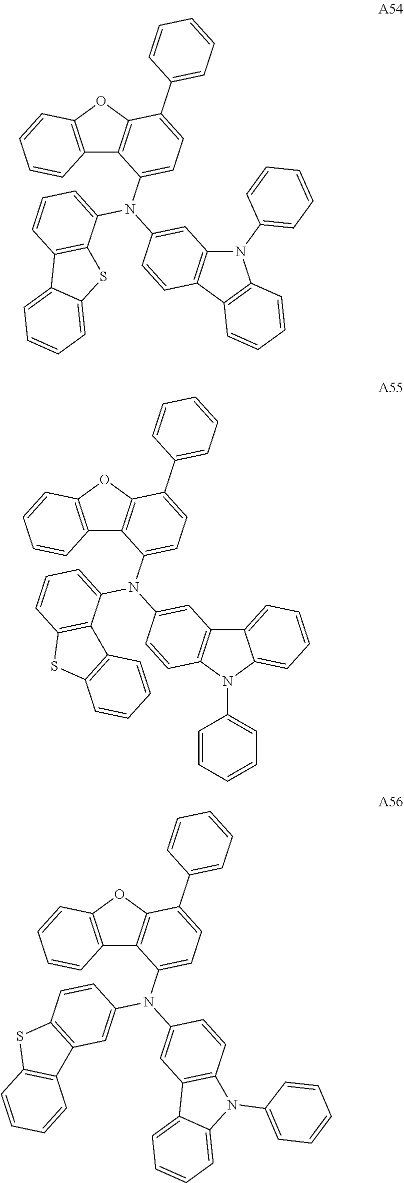

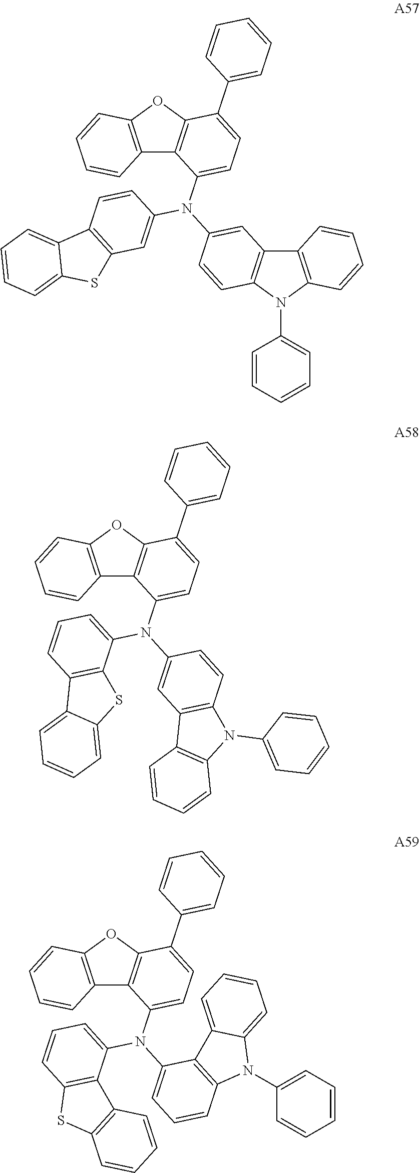

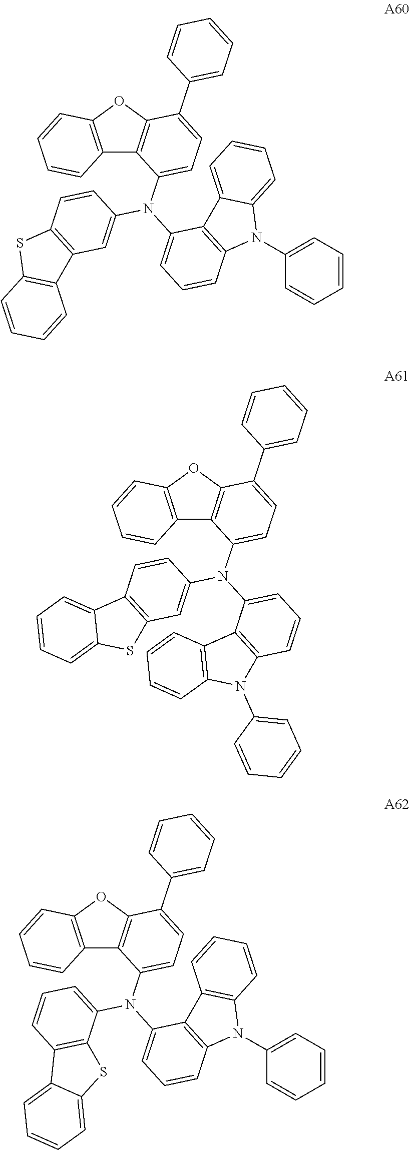 [SMILES: c1ccc(-c2ccc(N(c3ccc4sc5ccccc5c4c3)c3cccc4c3c3ccccc3n4-c3ccccc3)c3c2oc2ccccc23)cc1 ; c1ccc(-c2ccc(N(c3ccc4c(c3)sc3ccccc34)c3cccc4c3c3ccccc3n4-c3ccccc3)c3c2oc2ccccc23)cc1 ; c1ccc(-c2ccc(N(c3cccc4c3sc3ccccc34)c3cccc4c3c3ccccc3n4-c3ccccc3)c3c2oc2ccccc23)cc1]

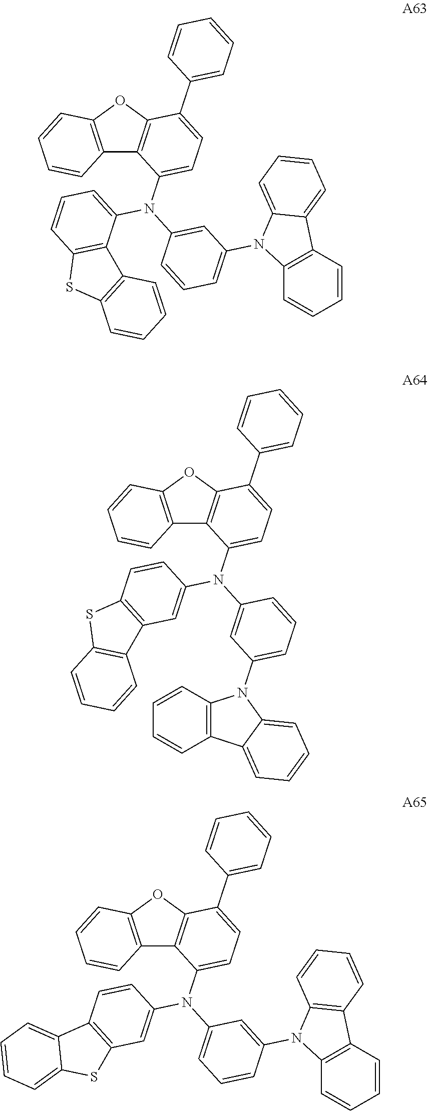

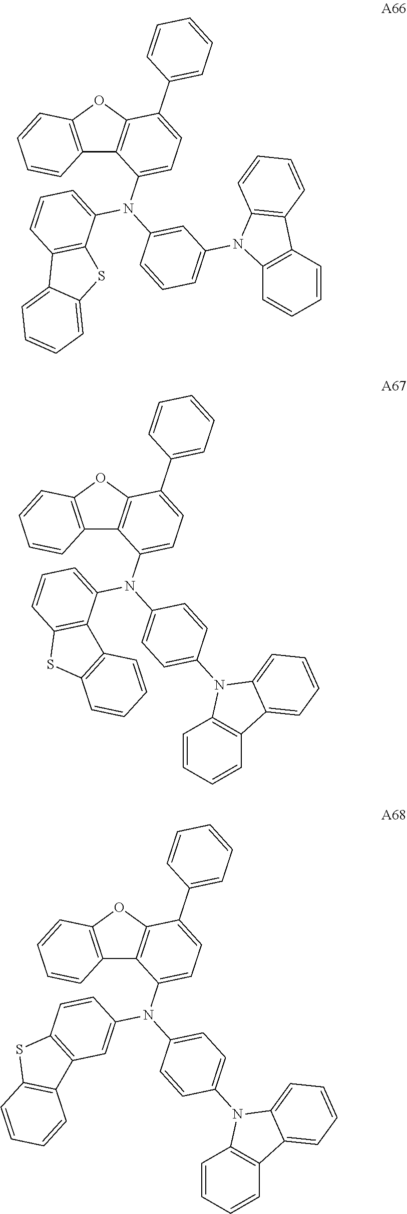

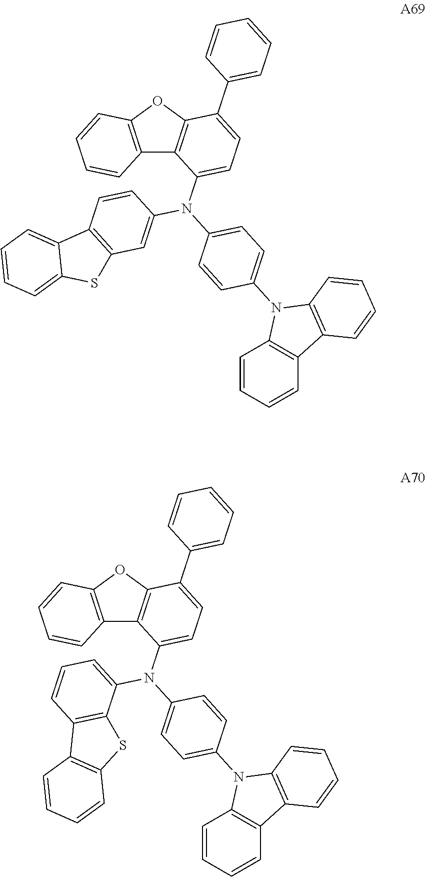 [SMILES: c1ccc(-c2ccc(N(c3ccc(-n4c5ccccc5c5ccccc54)cc3)c3ccc4c(c3)sc3ccccc34)c3c2oc2ccccc23)cc1 ; c1ccc(-c2ccc(N(c3ccc(-n4c5ccccc5c5ccccc54)cc3)c3cccc4c3sc3ccccc34)c3c2oc2ccccc23)cc1]

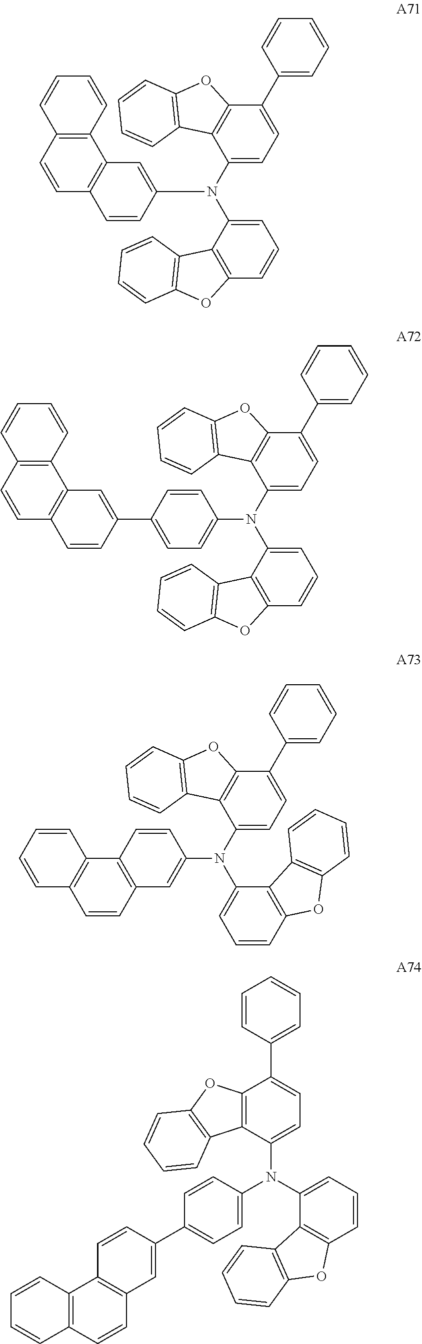

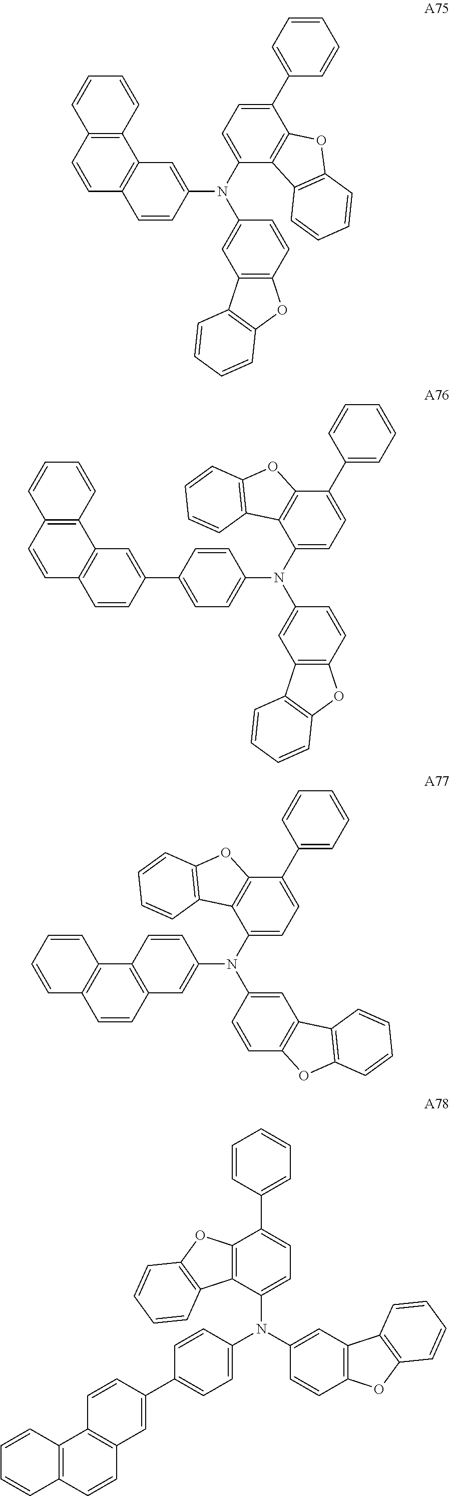

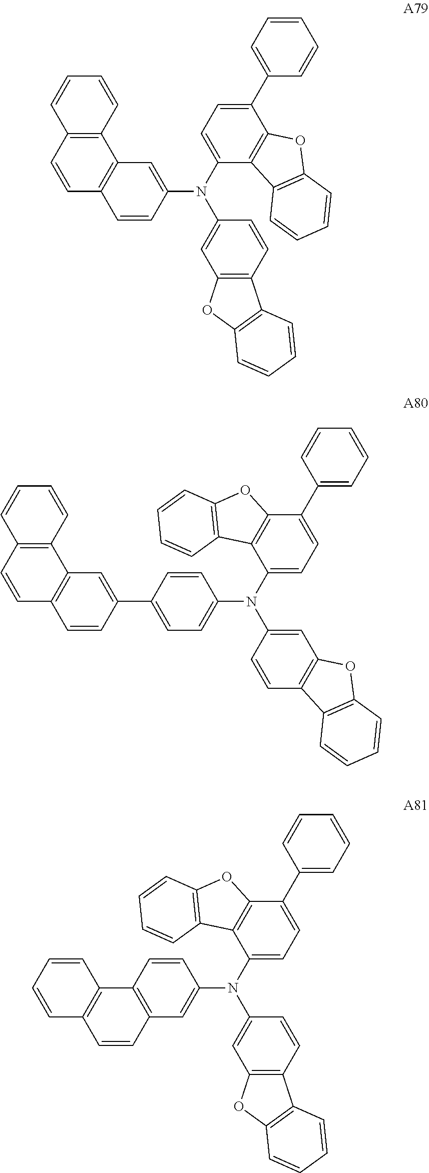 [SMILES: c1ccc(-c2ccc(N(c3ccc4c(c3)oc3ccccc34)c3ccc4ccc5ccccc5c4c3)c3c2oc2ccccc23)cc1 ; c1ccc(-c2ccc(N(c3ccc(-c4ccc5ccc6ccccc6c5c4)cc3)c3ccc4c(c3)oc3ccccc34)c3c2oc2ccccc23)cc1 ; c1ccc(-c2ccc(N(c3ccc4c(ccc5ccccc54)c3)c3ccc4c(c3)oc3ccccc34)c3c2oc2ccccc23)cc1]

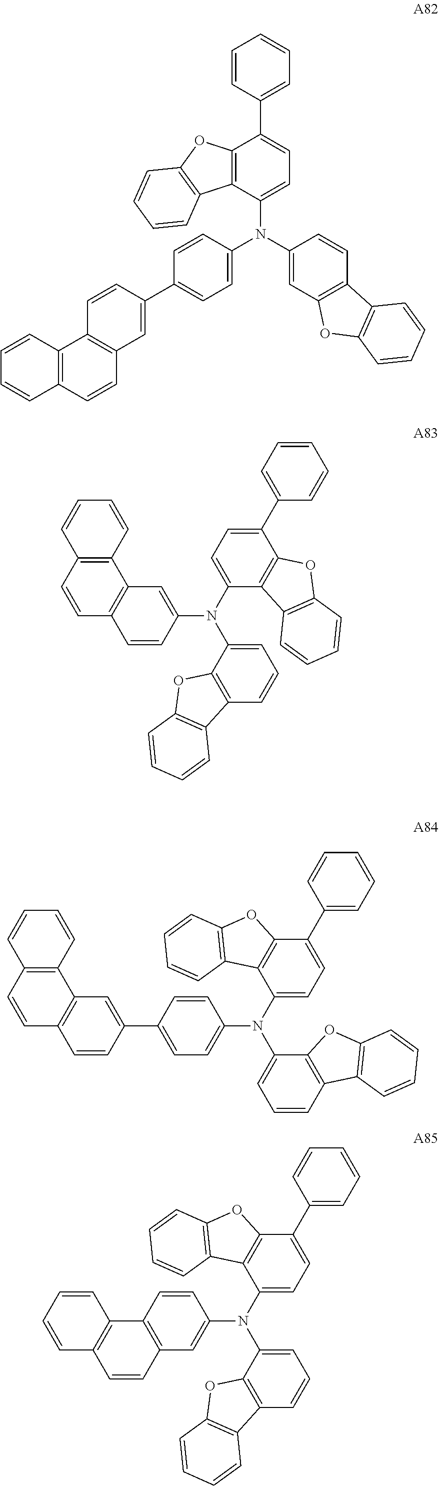

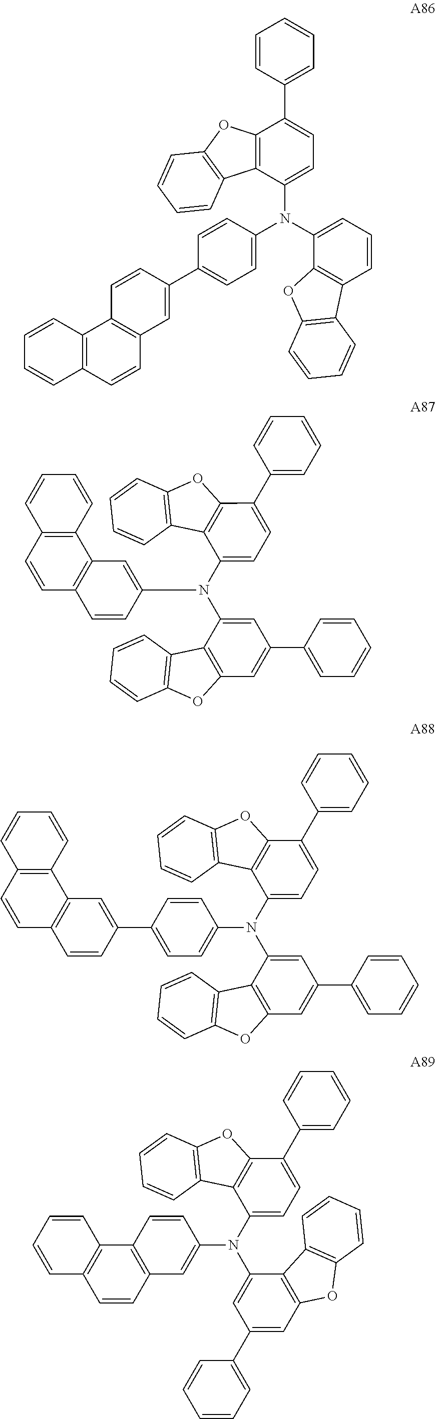

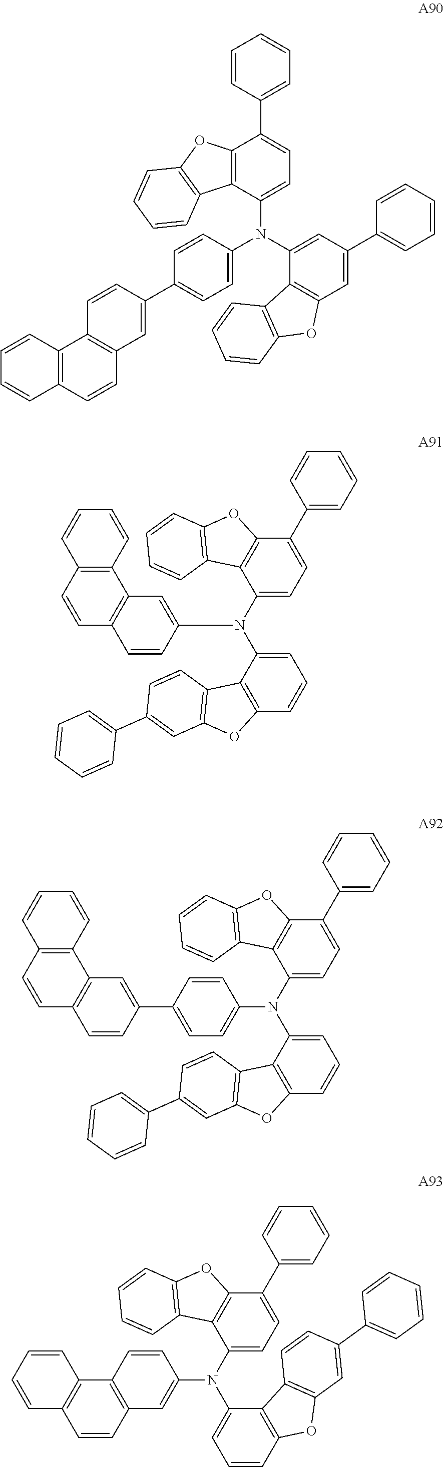 [SMILES: c1ccc(-c2cc(N(c3ccc(-c4ccc5c(ccc6ccccc65)c4)cc3)c3ccc(-c4ccccc4)c4oc5ccccc5c34)c3c(c2)oc2ccccc23)cc1 ; c1ccc(-c2ccc3c(c2)oc2cccc(N(c4ccc5ccc6ccccc6c5c4)c4ccc(-c5ccccc5)c5oc6ccccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5oc6ccccc6c45)c23)cc1 ; c1ccc(-c2ccc3c(c2)oc2cccc(N(c4ccc5c(ccc6ccccc65)c4)c4ccc(-c5ccccc5)c5oc6ccccc6c45)c23)cc1]

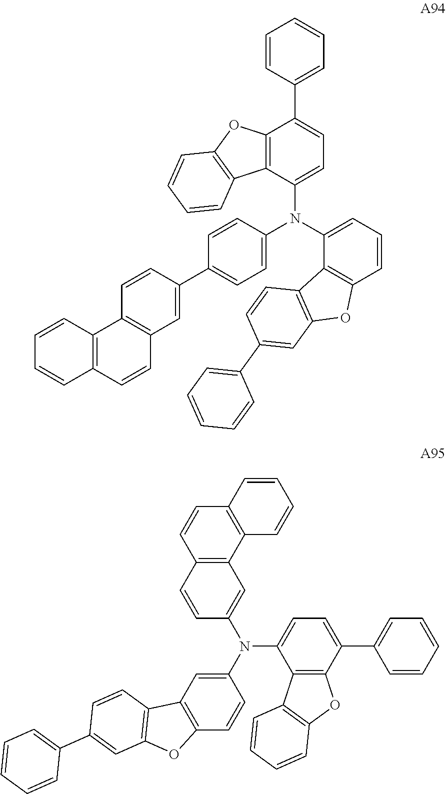 [SMILES: c1ccc(-c2ccc3c(c2)oc2cccc(N(c4ccc(-c5ccc6c(ccc7ccccc76)c5)cc4)c4ccc(-c5ccccc5)c5oc6ccccc6c45)c23)cc1 ; c1ccc(-c2ccc3c(c2)oc2ccc(N(c4ccc5ccc6ccccc6c5c4)c4ccc(-c5ccccc5)c5oc6ccccc6c45)cc23)cc1]

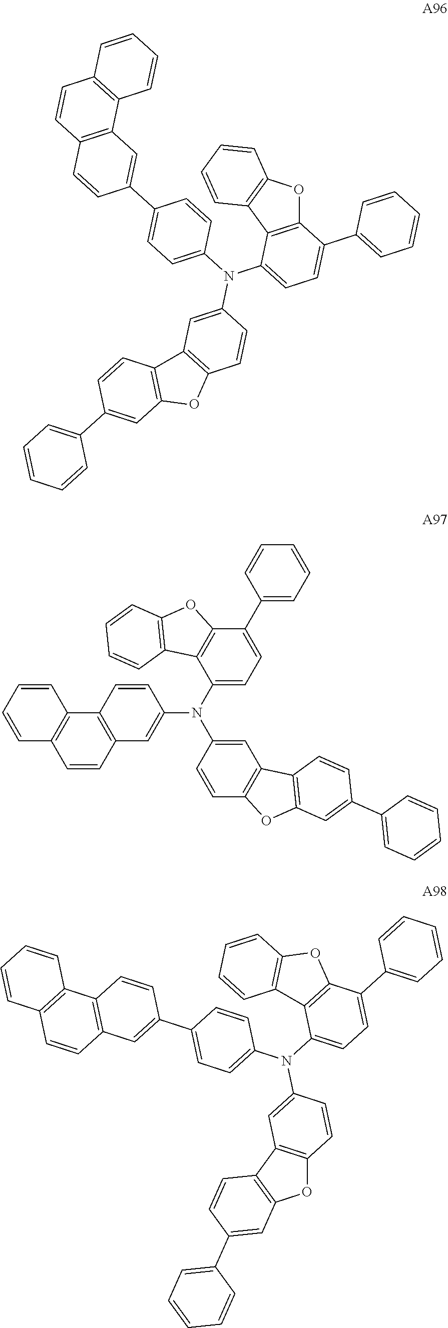 [SMILES: c1ccc(-c2ccc3c(c2)oc2ccc(N(c4ccc(-c5ccc6ccc7ccccc7c6c5)cc4)c4ccc(-c5ccccc5)c5oc6ccccc6c45)cc23)cc1 ; c1ccc(-c2ccc3c(c2)oc2ccc(N(c4ccc5c(ccc6ccccc65)c4)c4ccc(-c5ccccc5)c5oc6ccccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5oc6ccccc6c45)cc23)cc1]

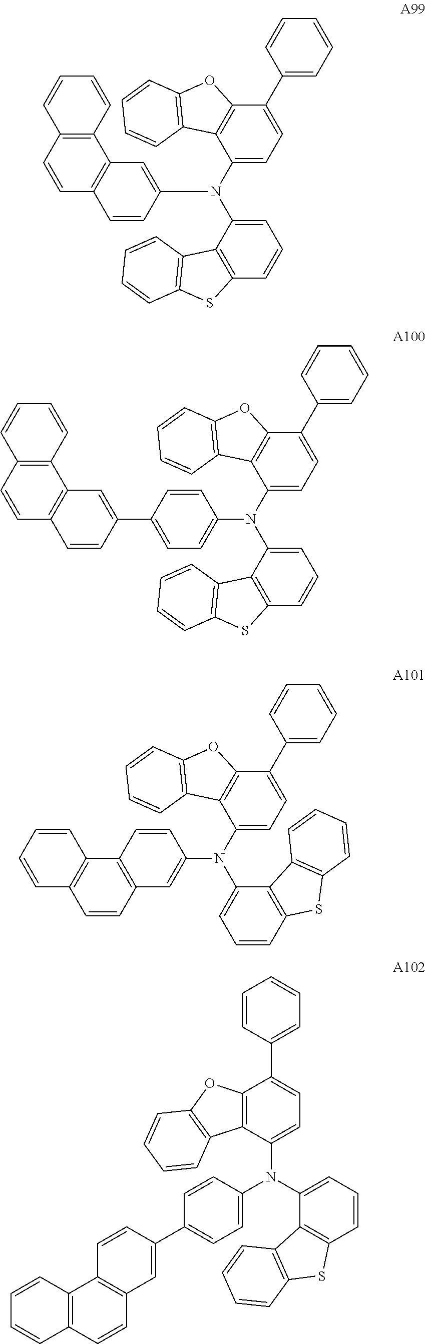

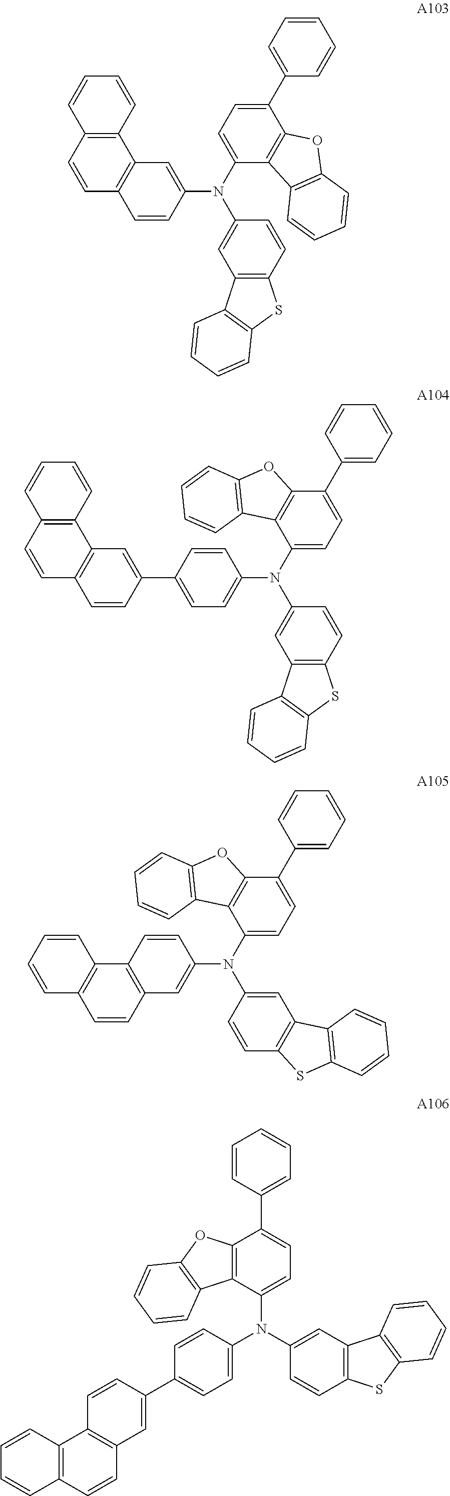

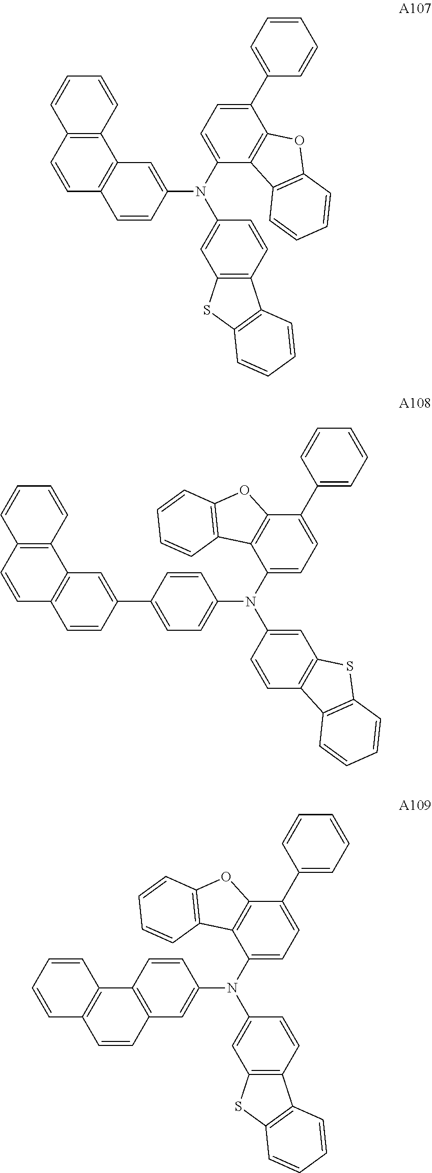 [SMILES: c1ccc(-c2ccc(N(c3ccc4c(c3)sc3ccccc34)c3ccc4ccc5ccccc5c4c3)c3c2oc2ccccc23)cc1 ; c1ccc(-c2ccc(N(c3ccc(-c4ccc5ccc6ccccc6c5c4)cc3)c3ccc4c(c3)sc3ccccc34)c3c2oc2ccccc23)cc1 ; c1ccc(-c2ccc(N(c3ccc4c(ccc5ccccc54)c3)c3ccc4c(c3)sc3ccccc34)c3c2oc2ccccc23)cc1]

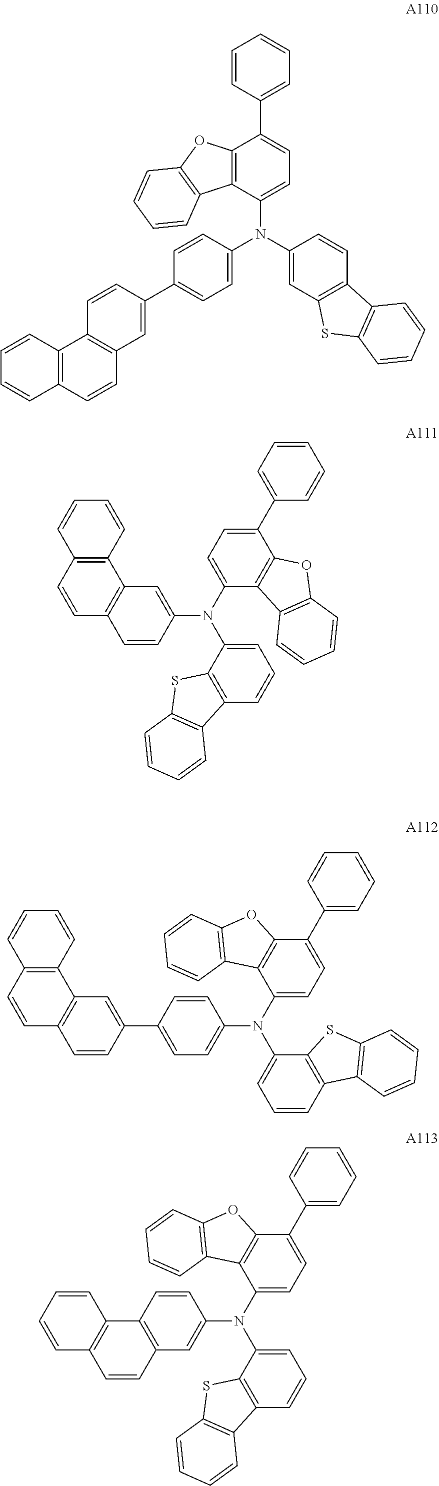

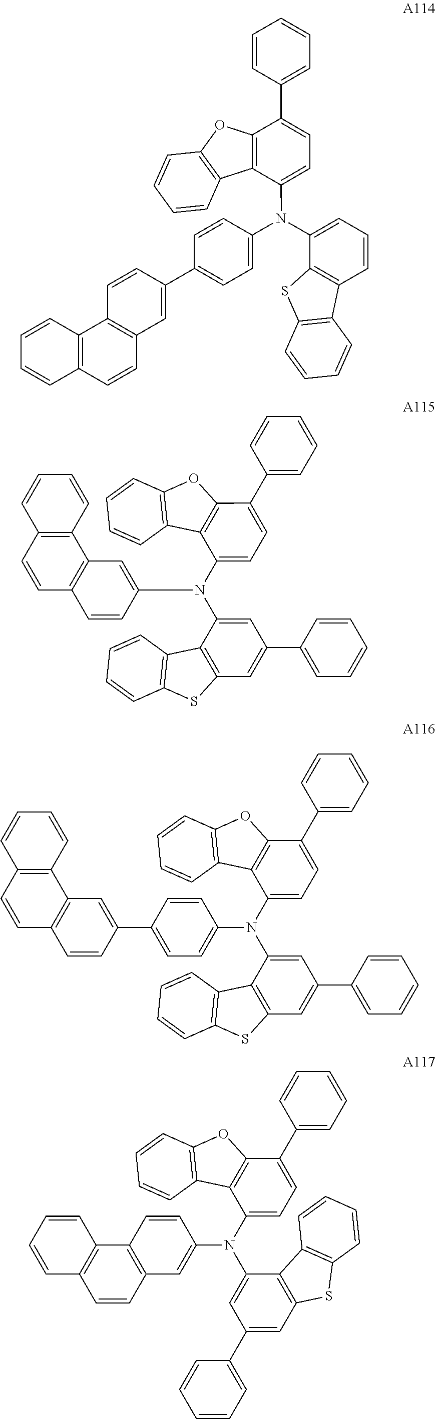

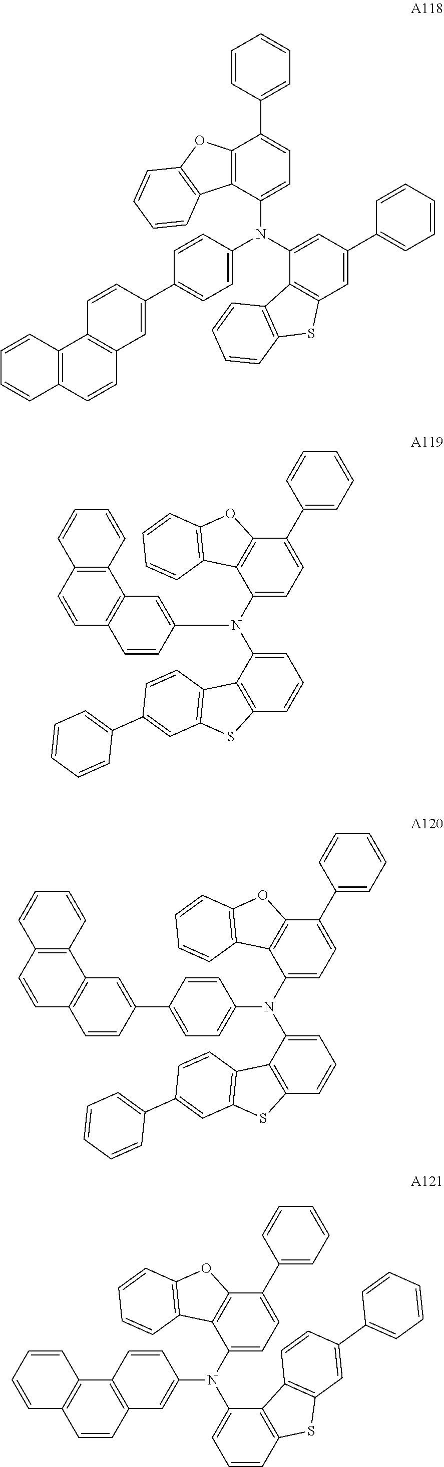 [SMILES: c1ccc(-c2cc(N(c3ccc(-c4ccc5c(ccc6ccccc65)c4)cc3)c3ccc(-c4ccccc4)c4oc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5oc6ccccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5oc6ccccc6c45)c23)cc1 ; c1ccc(-c2ccc3c(c2)sc2cccc(N(c4ccc5c(ccc6ccccc65)c4)c4ccc(-c5ccccc5)c5oc6ccccc6c45)c23)cc1]

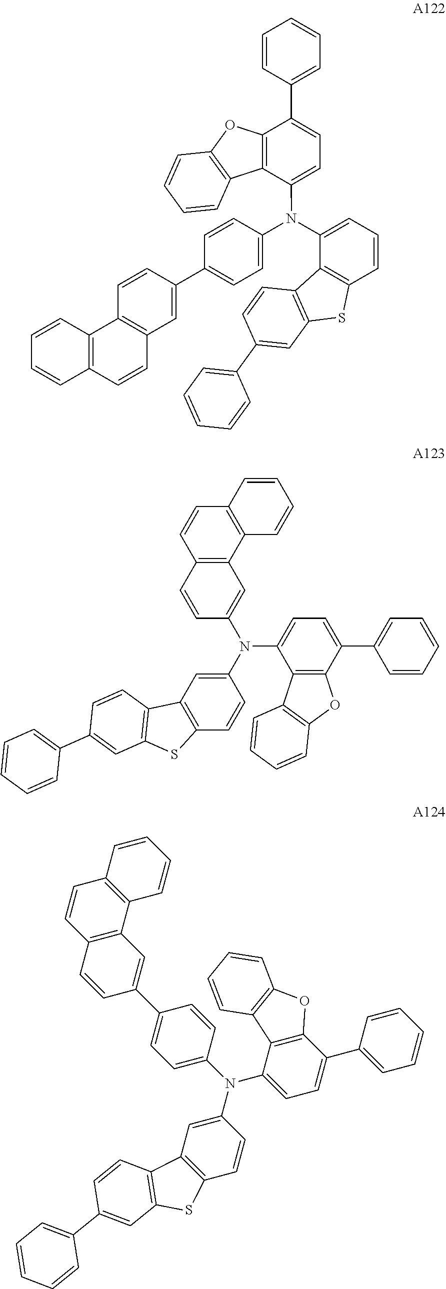 [SMILES: c1ccc(-c2ccc3c(c2)sc2cccc(N(c4ccc(-c5ccc6c(ccc7ccccc76)c5)cc4)c4ccc(-c5ccccc5)c5oc6ccccc6c45)c23)cc1 ; c1ccc(-c2ccc3c(c2)sc2ccc(N(c4ccc5ccc6ccccc6c5c4)c4ccc(-c5ccccc5)c5oc6ccccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5oc6ccccc6c45)cc23)cc1]

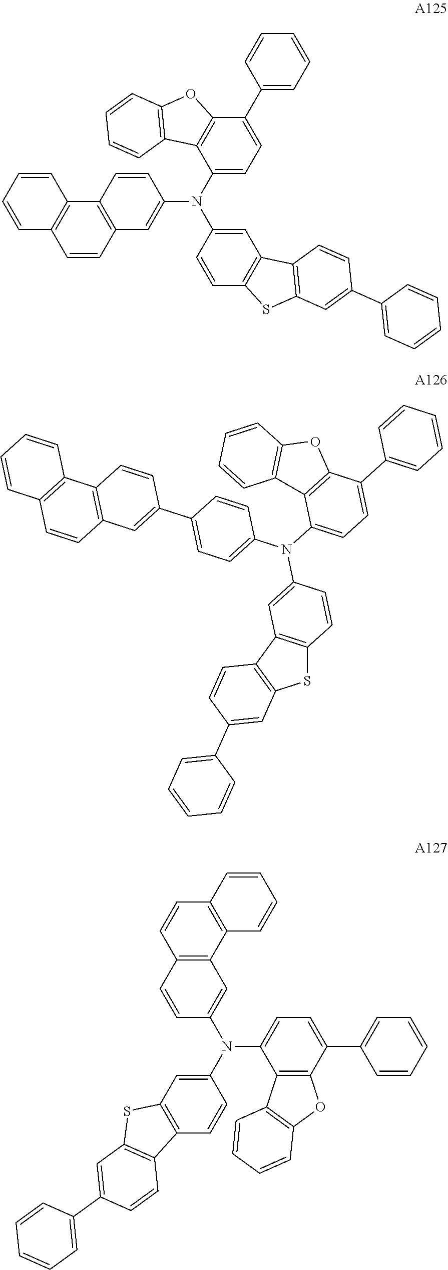 [SMILES: c1ccc(-c2ccc3c(c2)sc2ccc(N(c4ccc5c(ccc6ccccc65)c4)c4ccc(-c5ccccc5)c5oc6ccccc6c45)cc23)cc1 ; c1ccc(-c2ccc3c(c2)sc2ccc(N(c4ccc(-c5ccc6c(ccc7ccccc76)c5)cc4)c4ccc(-c5ccccc5)c5oc6ccccc6c45)cc23)cc1 ; c1ccc(-c2ccc3c(c2)sc2cc(N(c4ccc5ccc6ccccc6c5c4)c4ccc(-c5ccccc5)c5oc6ccccc6c45)ccc23)cc1]

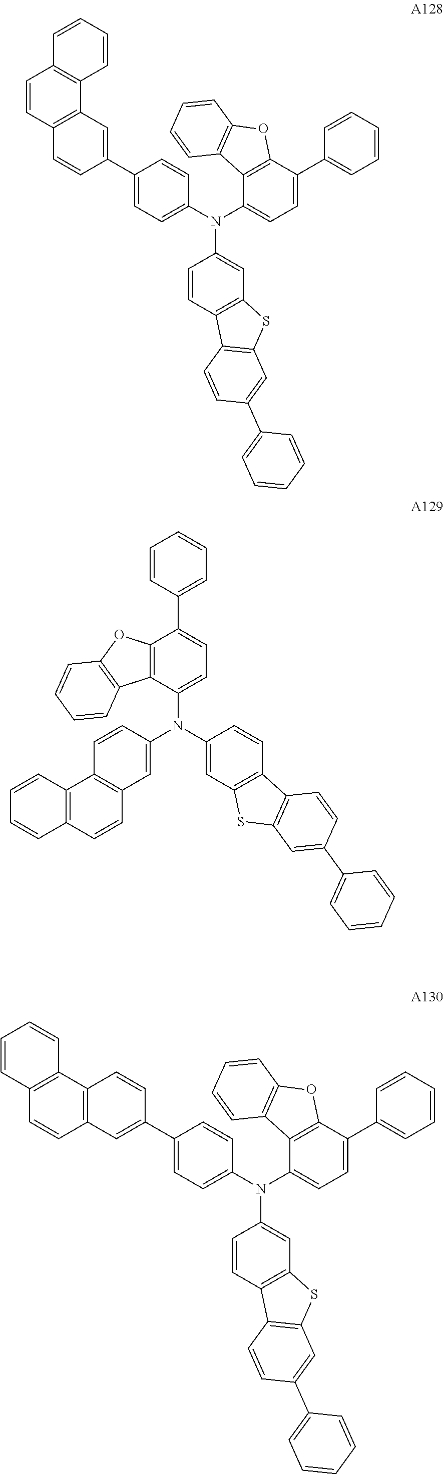 [SMILES: c1ccc(-c2ccc3c(c2)sc2cc(N(c4ccc(-c5ccc6ccc7ccccc7c6c5)cc4)c4ccc(-c5ccccc5)c5oc6ccccc6c45)ccc23)cc1 ; c1ccc(-c2ccc3c(c2)sc2cc(N(c4ccc5c(ccc6ccccc65)c4)c4ccc(-c5ccccc5)c5oc6ccccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5oc6ccccc6c45)ccc23)cc1]

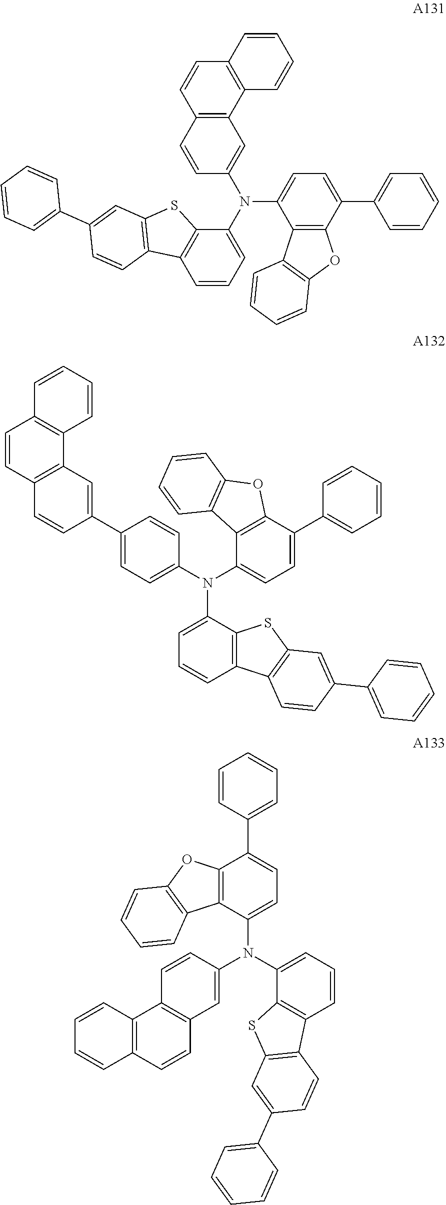 [SMILES: c1ccc(-c2ccc3c(c2)sc2c(N(c4ccc5ccc6ccccc6c5c4)c4ccc(-c5ccccc5)c5oc6ccccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5oc6ccccc6c45)cccc23)cc1 ; c1ccc(-c2ccc3c(c2)sc2c(N(c4ccc5c(ccc6ccccc65)c4)c4ccc(-c5ccccc5)c5oc6ccccc6c45)cccc23)cc1]

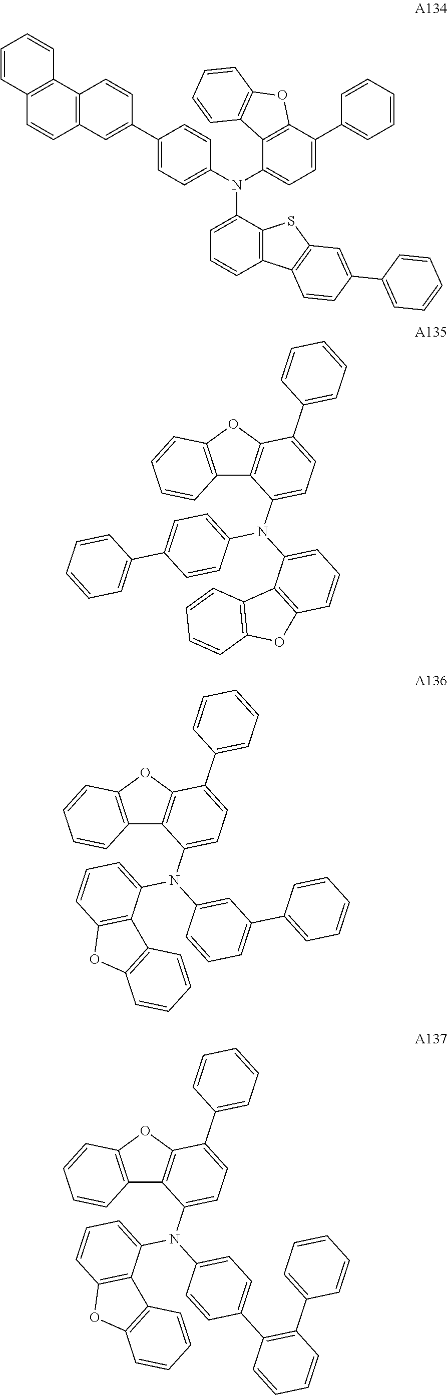

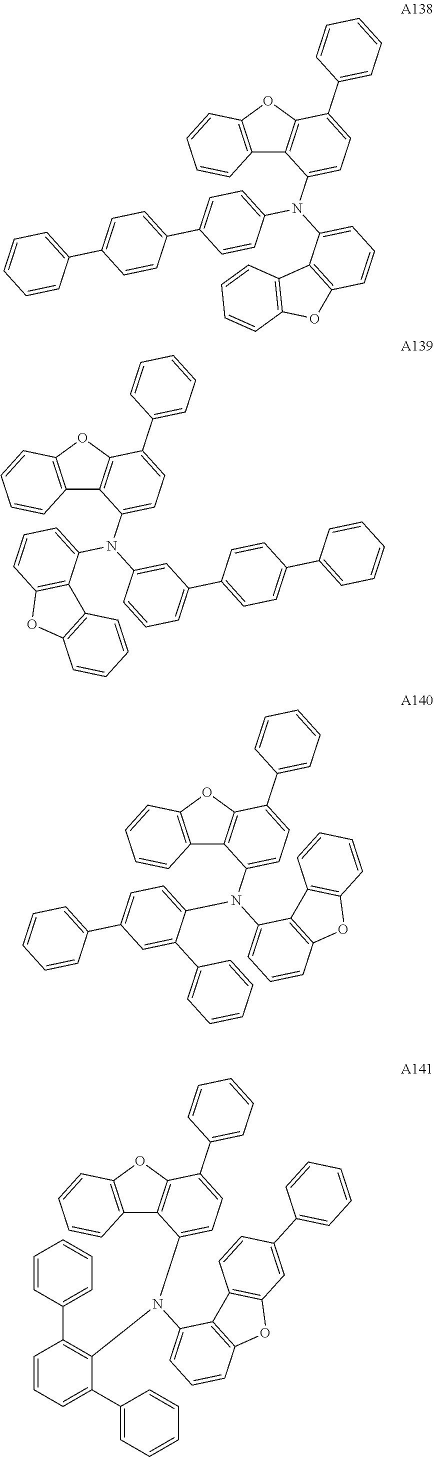 [SMILES: c1ccc(-c2ccc(-c3ccc(N(c4cccc5oc6ccccc6c45)c4ccc(-c5ccccc5)c5oc6ccccc6c45)cc3)cc2)cc1 ; c1ccc(-c2ccc(-c3cccc(N(c4cccc5oc6ccccc6c45)c4ccc(-c5ccccc5)c5oc6ccccc6c45)c3)cc2)cc1 ; c1ccc(-c2ccc(N(c3cccc4oc5ccccc5c34)c3ccc(-c4ccccc4)c4oc5ccccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5oc6ccccc6c45)c23)cc1]

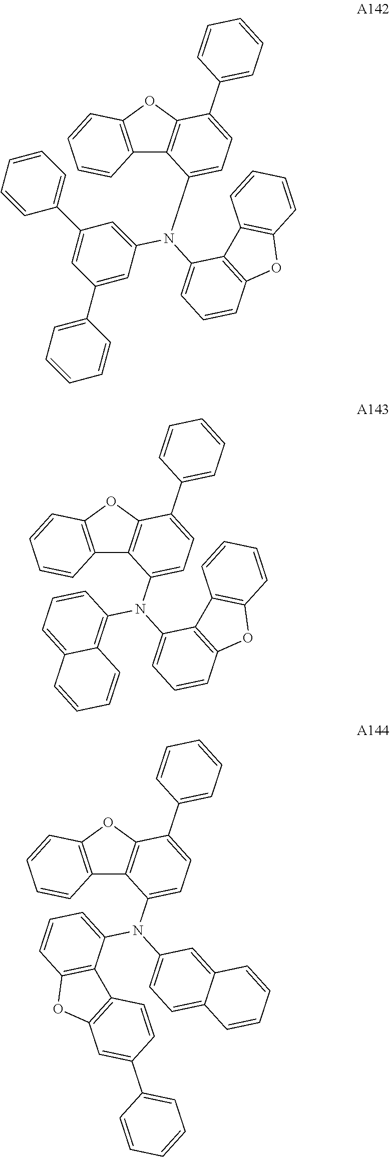 [SMILES: c1ccc(-c2cc(-c3ccccc3)cc(N(c3cccc4oc5ccccc5c34)c3ccc(-c4ccccc4)c4oc5ccccc5c34)c2)cc1 ; c1ccc(-c2ccc(N(c3cccc4ccccc34)c3cccc4oc5ccccc5c34)c3c2oc2ccccc23)cc1 ; c1ccc(-c2ccc3c(c2)oc2cccc(N(c4ccc5ccccc5c4)c4ccc(-c5ccccc5)c5oc6ccccc6c45)c23)cc1]

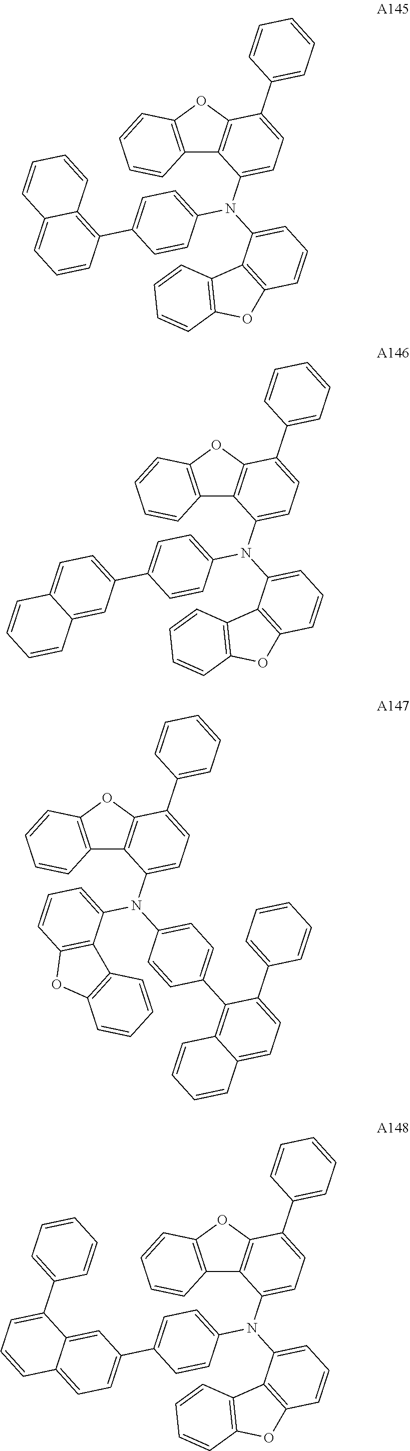

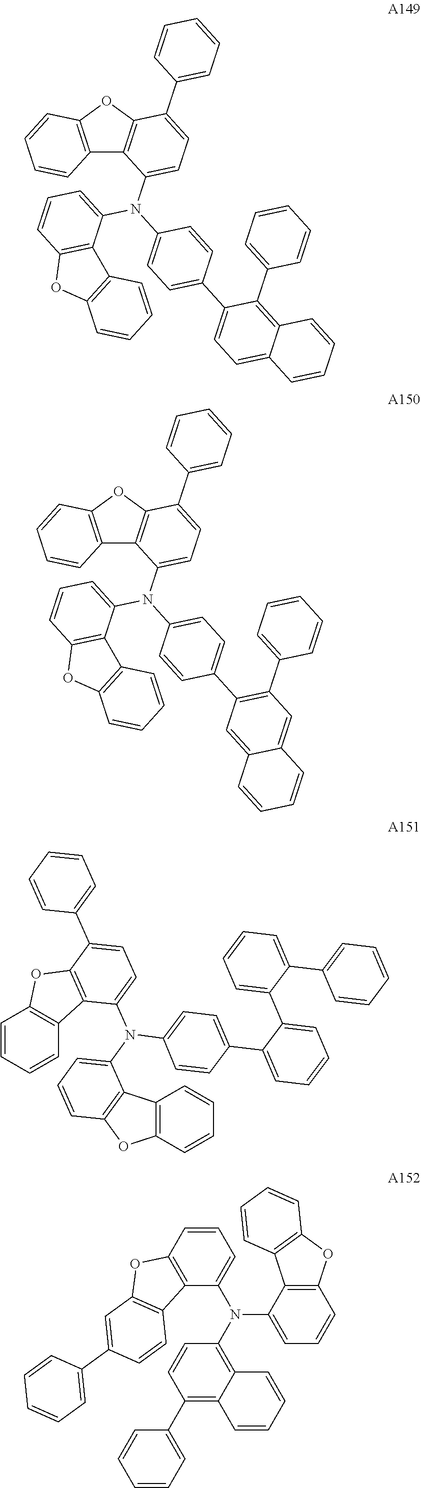 [SMILES: c1ccc(-c2c(-c3ccc(N(c4cccc5oc6ccccc6c45)c4ccc(-c5ccccc5)c5oc6ccccc6c45)cc3)ccc3ccccc23)cc1 ; c1ccc(-c2cc3ccccc3cc2-c2ccc(N(c3cccc4oc5ccccc5c34)c3ccc(-c4ccccc4)c4oc5ccccc5c34)cc2)cc1 ; c1ccc(-c2ccccc2-c2ccccc2-c2ccc(N(c3cccc4oc5ccccc5c34)c3ccc(-c4ccccc4)c4oc5ccccc5c34)cc2)cc1 ; c1ccc(-c2ccc3c(c2)oc2cccc(N(c4ccc(-c5ccccc5)c5ccccc45)c4cccc5oc6ccccc6c45)c23)cc1]

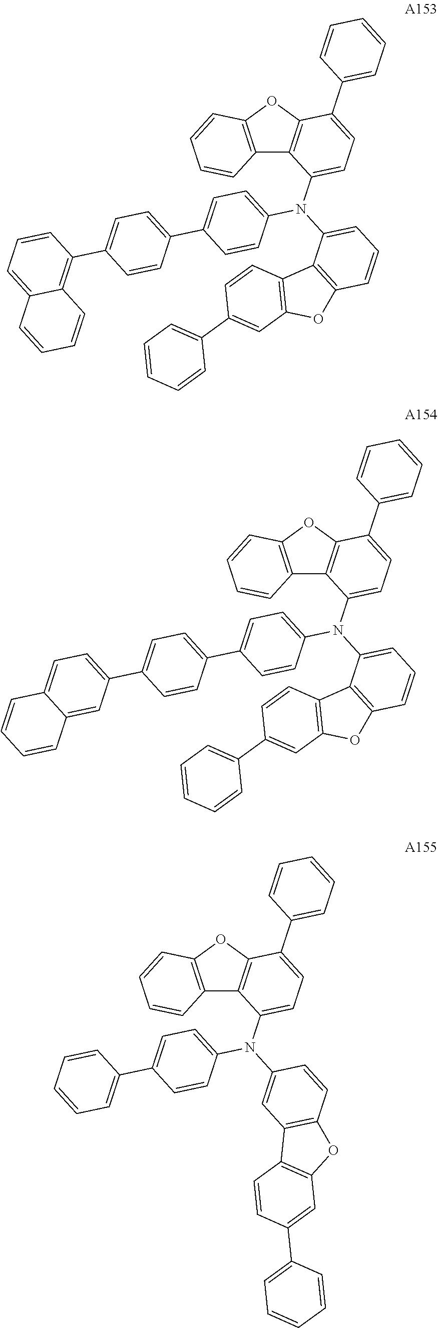 [SMILES: c1ccc(-c2ccc3c(c2)oc2cccc(N(c4ccc(-c5ccc(-c6cccc7ccccc67)cc5)cc4)c4ccc(-c5ccccc5)c5oc6ccccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4oc5cc(-c6ccccc6)ccc5c4c3)c3ccc(-c4ccccc4)c4oc5ccccc5c34)cc2)cc1]

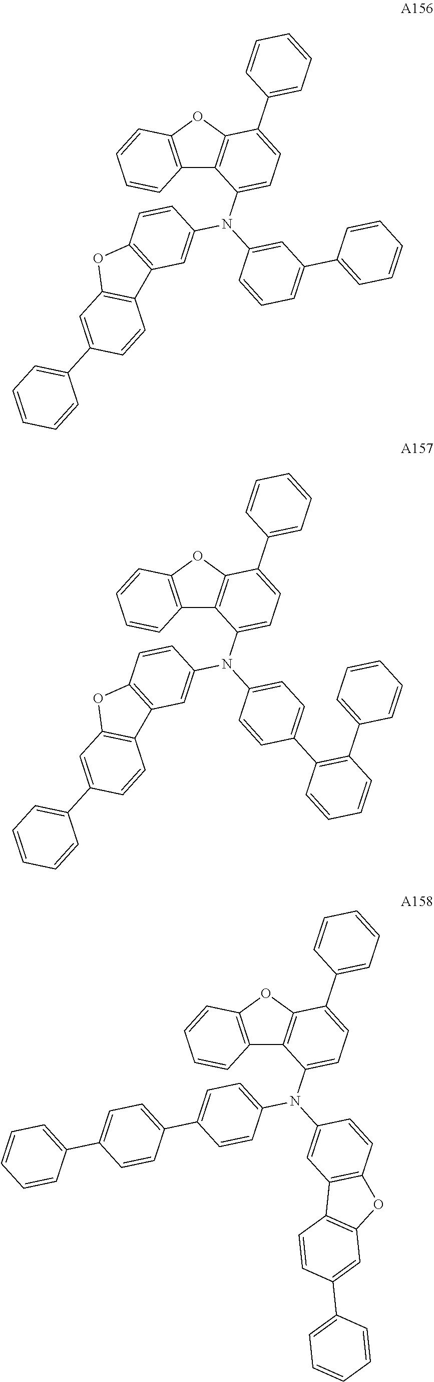 [SMILES: c1ccc(-c2cccc(N(c3ccc4oc5cc(-c6ccccc6)ccc5c4c3)c3ccc(-c4ccccc4)c4oc5ccccc5c34)c2)cc1 ; c1ccc(-c2ccc3c(c2)oc2ccc(N(c4ccc(-c5ccccc5-c5ccccc5)cc4)c4ccc(-c5ccccc5)c5oc6ccccc6c45)cc23)cc1 ; c1ccc(-c2ccc(-c3ccc(N(c4ccc5oc6cc(-c7ccccc7)ccc6c5c4)c4ccc(-c5ccccc5)c5oc6ccccc6c45)cc3)cc2)cc1]

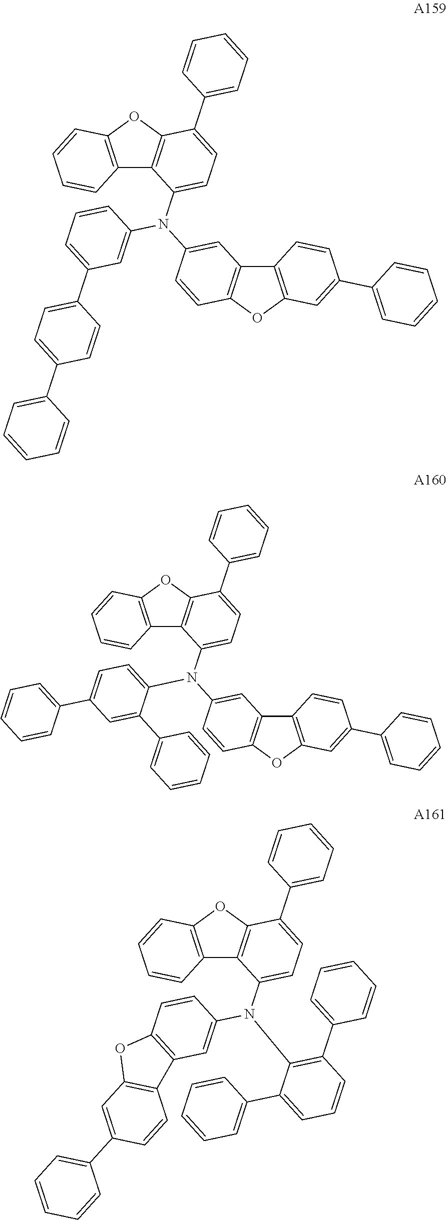 [SMILES: c1ccc(-c2ccc(-c3cccc(N(c4ccc5oc6cc(-c7ccccc7)ccc6c5c4)c4ccc(-c5ccccc5)c5oc6ccccc6c45)c3)cc2)cc1 ; c1ccc(-c2ccc(N(c3ccc4oc5cc(-c6ccccc6)ccc5c4c3)c3ccc(-c4ccccc4)c4oc5ccccc5c34)c(-c3ccccc3)c2)cc1 ; c1ccc(-c2ccc3c(c2)oc2ccc(N(c4c(-c5ccccc5)cccc4-c4ccccc4)c4ccc(-c5ccccc5)c5oc6ccccc6c45)cc23)cc1]

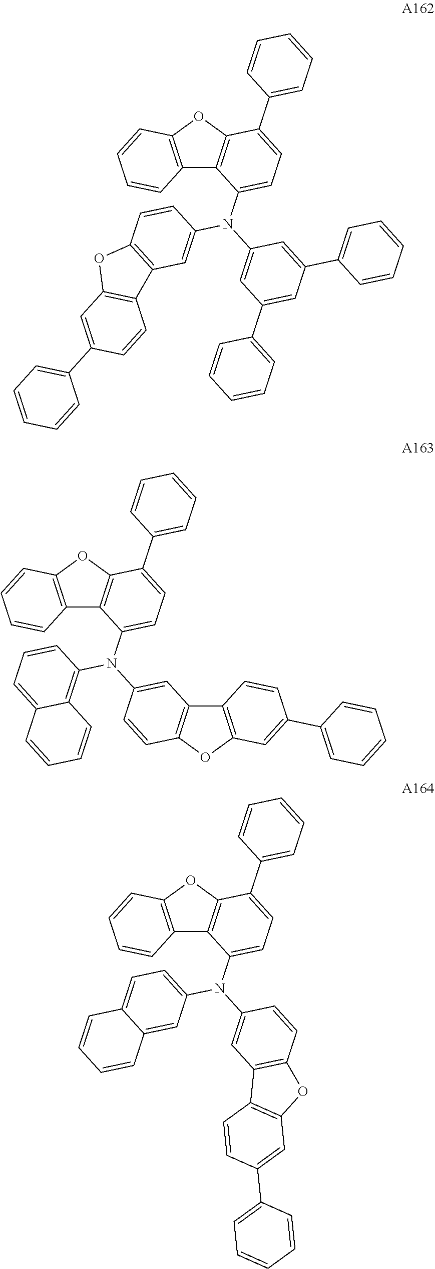 [SMILES: c1ccc(-c2cc(-c3ccccc3)cc(N(c3ccc4oc5cc(-c6ccccc6)ccc5c4c3)c3ccc(-c4ccccc4)c4oc5ccccc5c34)c2)cc1 ; c1ccc(-c2ccc3c(c2)oc2ccc(N(c4cccc5ccccc45)c4ccc(-c5ccccc5)c5oc6ccccc6c45)cc23)cc1 ; c1ccc(-c2ccc3c(c2)oc2ccc(N(c4ccc5ccccc5c4)c4ccc(-c5ccccc5)c5oc6ccccc6c45)cc23)cc1]

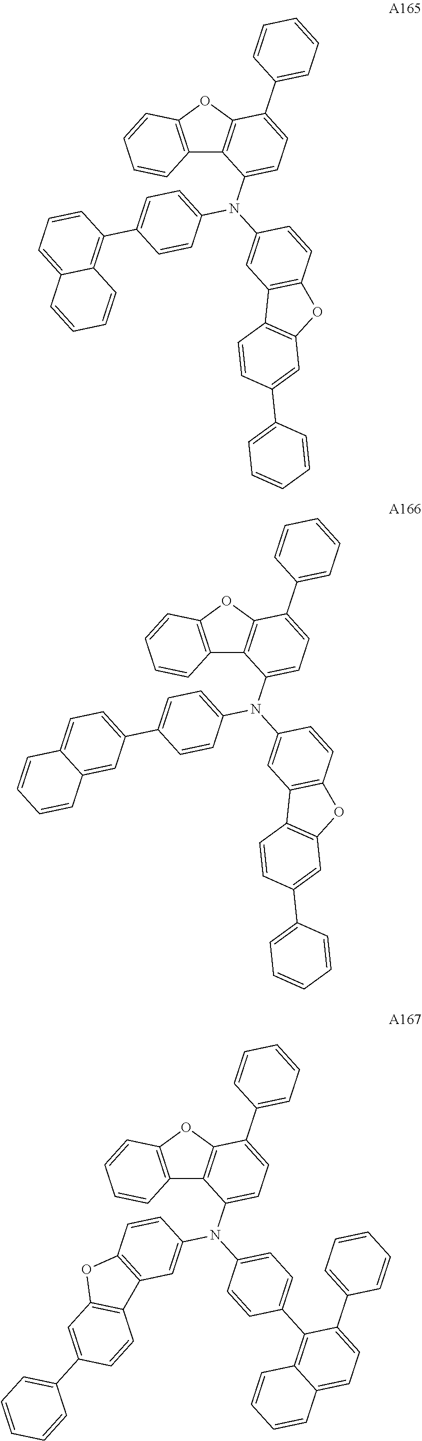

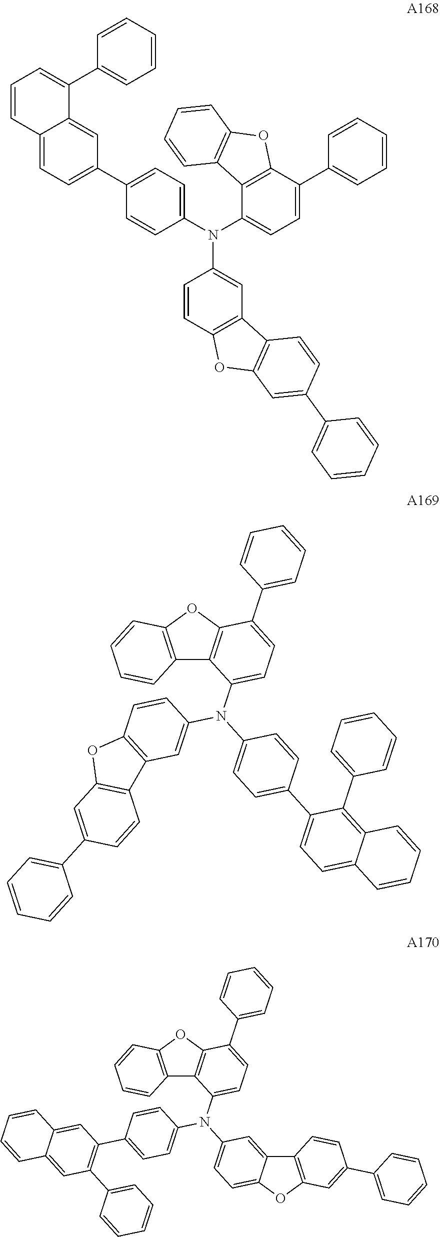 [SMILES: c1ccc(-c2ccc3c(c2)oc2ccc(N(c4ccc(-c5ccc6cccc(-c7ccccc7)c6c5)cc4)c4ccc(-c5ccccc5)c5oc6ccccc6c45)cc23)cc1 ; c1ccc(-c2ccc3c(c2)oc2ccc(N(c4ccc(-c5ccc6ccccc6c5-c5ccccc5)cc4)c4ccc(-c5ccccc5)c5oc6ccccc6c45)cc23)cc1 ; c1ccc(-c2ccc3c(c2)oc2ccc(N(c4ccc(-c5cc6ccccc6cc5-c5ccccc5)cc4)c4ccc(-c5ccccc5)c5oc6ccccc6c45)cc23)cc1]

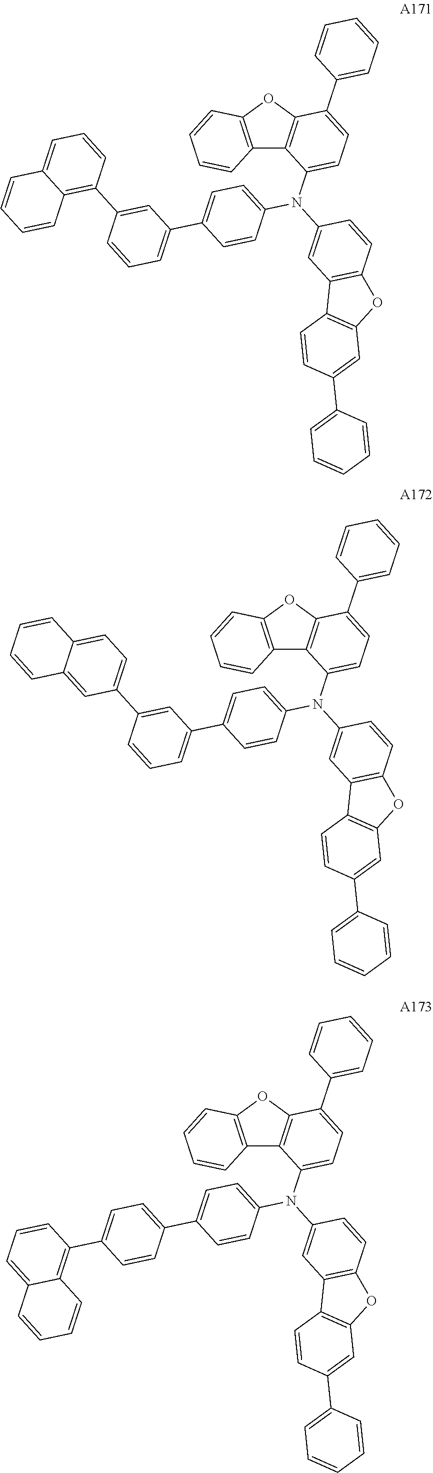

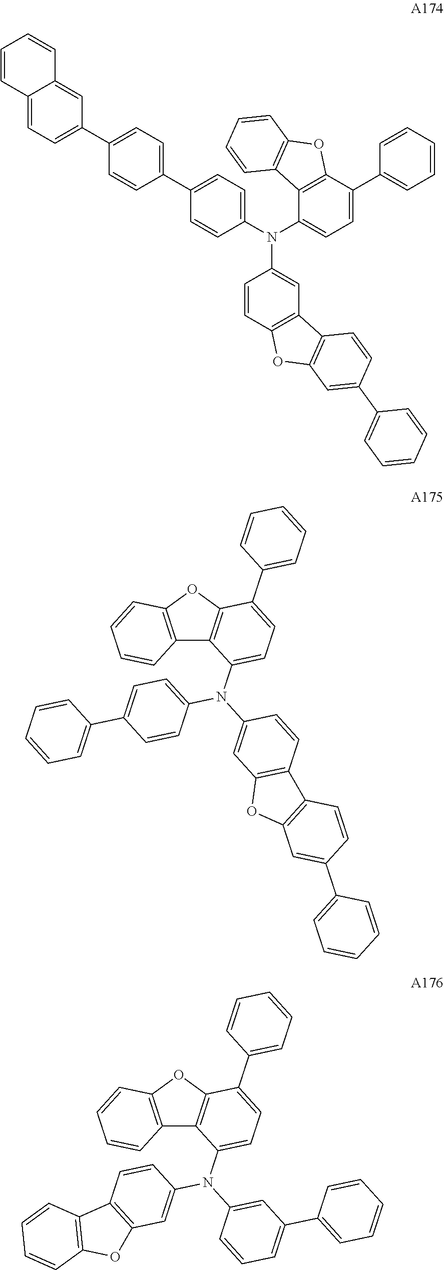 [SMILES: c1ccc(-c2ccc3c(c2)oc2ccc(N(c4ccc(-c5ccc(-c6ccc7ccccc7c6)cc5)cc4)c4ccc(-c5ccccc5)c5oc6ccccc6c45)cc23)cc1 ; c1ccc(-c2ccc(N(c3ccc4c(c3)oc3cc(-c5ccccc5)ccc34)c3ccc(-c4ccccc4)c4oc5ccccc5c34)cc2)cc1 ; c1ccc(-c2cccc(N(c3ccc4c(c3)oc3ccccc34)c3ccc(-c4ccccc4)c4oc5ccccc5c34)c2)cc1]

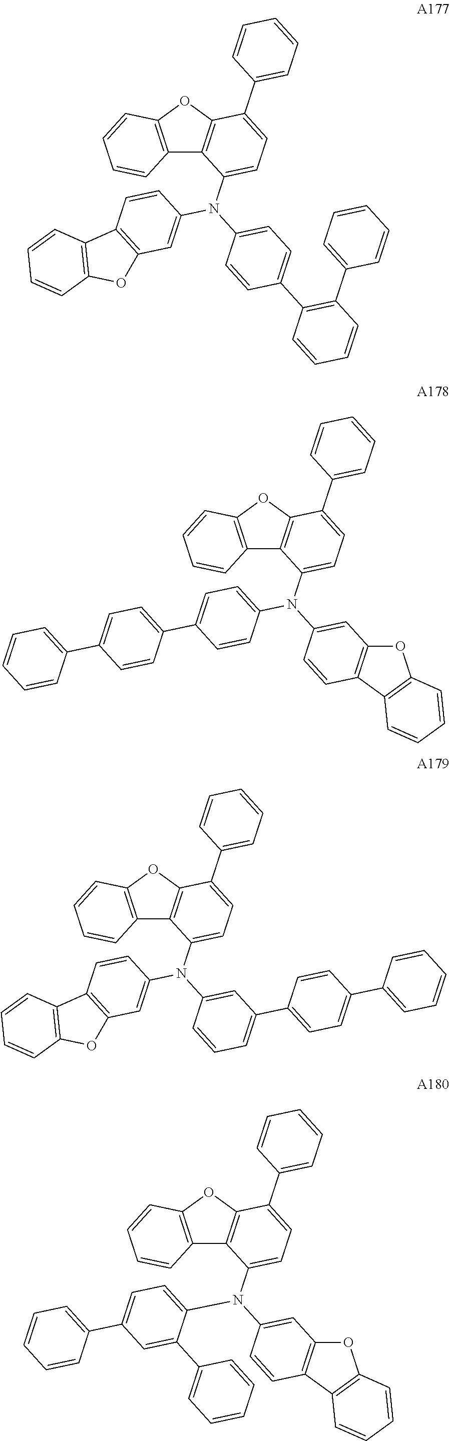 [SMILES: c1ccc(-c2ccccc2-c2ccc(N(c3ccc4c(c3)oc3ccccc34)c3ccc(-c4ccccc4)c4oc5ccccc5c34)cc2)cc1 ; c1ccc(-c2ccc(-c3ccc(N(c4ccc5c(c4)oc4ccccc45)c4ccc(-c5ccccc5)c5oc6ccccc6c45)cc3)cc2)cc1 ; c1ccc(-c2ccc(-c3cccc(N(c4ccc5c(c4)oc4ccccc45)c4ccc(-c5ccccc5)c5oc6ccccc6c45)c3)cc2)cc1 ; c1ccc(-c2ccc(N(c3ccc4c(c3)oc3ccccc34)c3ccc(-c4ccccc4)c4oc5ccccc5c34)c(-c3ccccc3)c2)cc1]

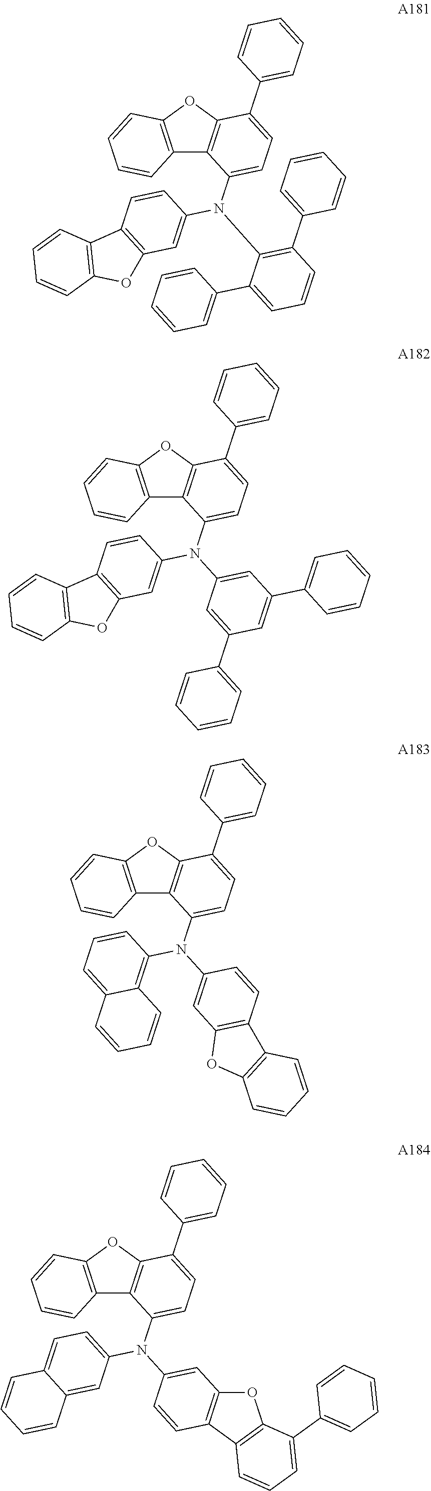

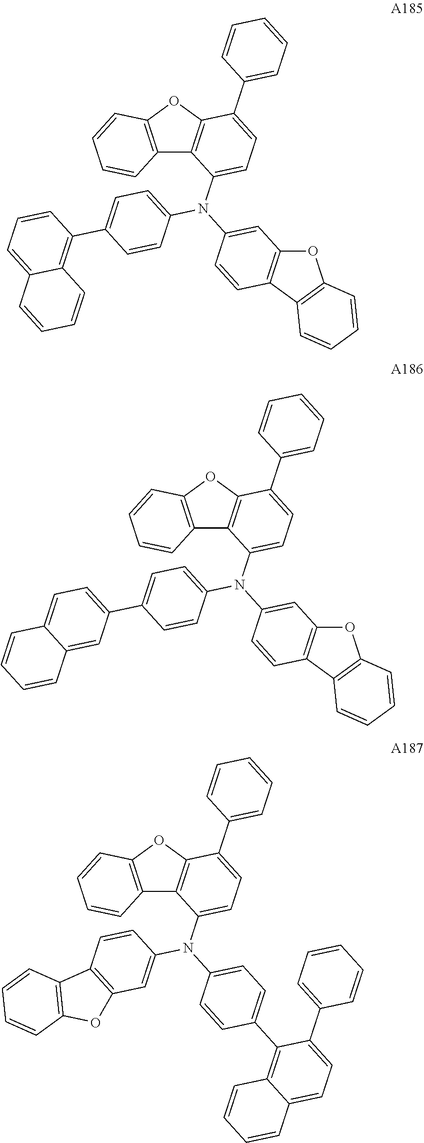 [SMILES: c1ccc(-c2ccc(N(c3ccc(-c4cccc5ccccc45)cc3)c3ccc4c(c3)oc3ccccc34)c3c2oc2ccccc23)cc1 ; c1ccc(-c2ccc(N(c3ccc(-c4ccc5ccccc5c4)cc3)c3ccc4c(c3)oc3ccccc34)c3c2oc2ccccc23)cc1 ; c1ccc(-c2ccc3ccccc3c2-c2ccc(N(c3ccc4c(c3)oc3ccccc34)c3ccc(-c4ccccc4)c4oc5ccccc5c34)cc2)cc1]

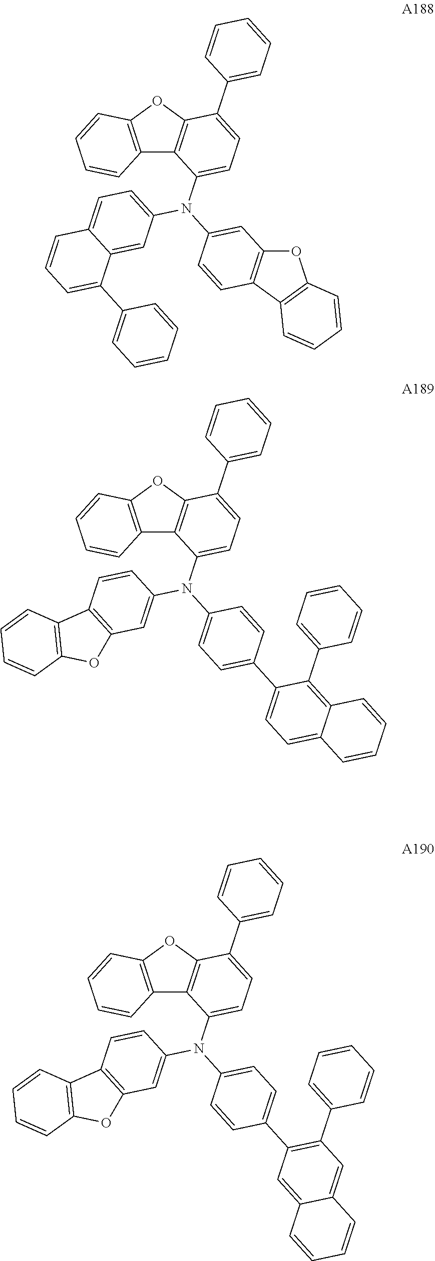 [SMILES: c1ccc(-c2cccc3ccc(N(c4ccc5c(c4)oc4ccccc45)c4ccc(-c5ccccc5)c5oc6ccccc6c45)cc23)cc1 ; c1ccc(-c2c(-c3ccc(N(c4ccc5c(c4)oc4ccccc45)c4ccc(-c5ccccc5)c5oc6ccccc6c45)cc3)ccc3ccccc23)cc1 ; c1ccc(-c2cc3ccccc3cc2-c2ccc(N(c3ccc4c(c3)oc3ccccc34)c3ccc(-c4ccccc4)c4oc5ccccc5c34)cc2)cc1]

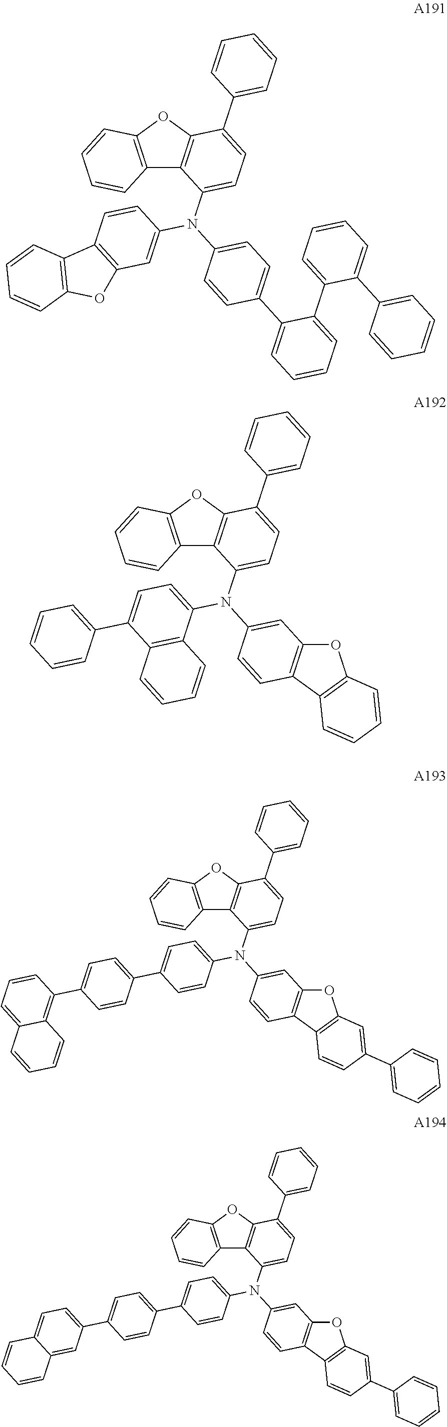

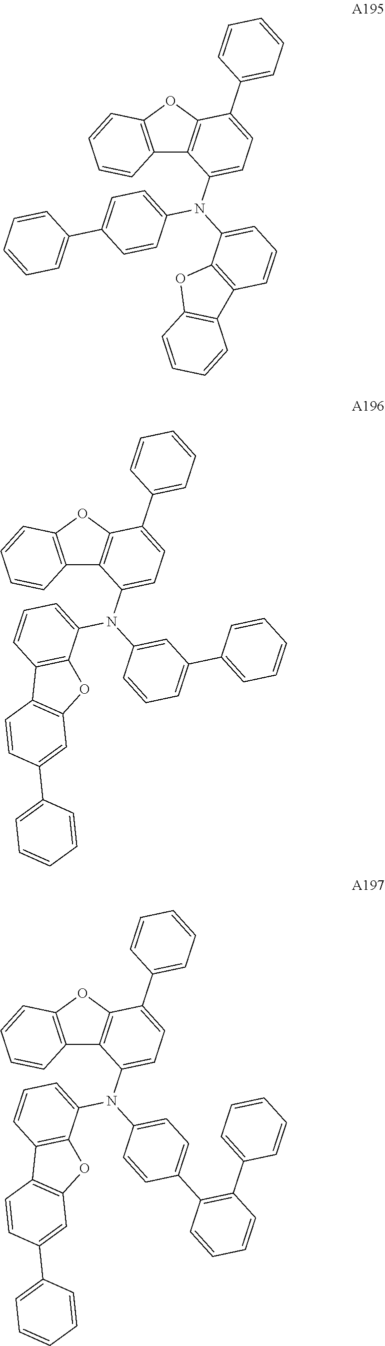 [SMILES: c1ccc(-c2ccc(N(c3cccc4c3oc3ccccc34)c3ccc(-c4ccccc4)c4oc5ccccc5c34)cc2)cc1 ; c1ccc(-c2cccc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4ccc(-c5ccccc5-c5ccccc5)cc4)c4ccc(-c5ccccc5)c5oc6ccccc6c45)cccc23)cc1]

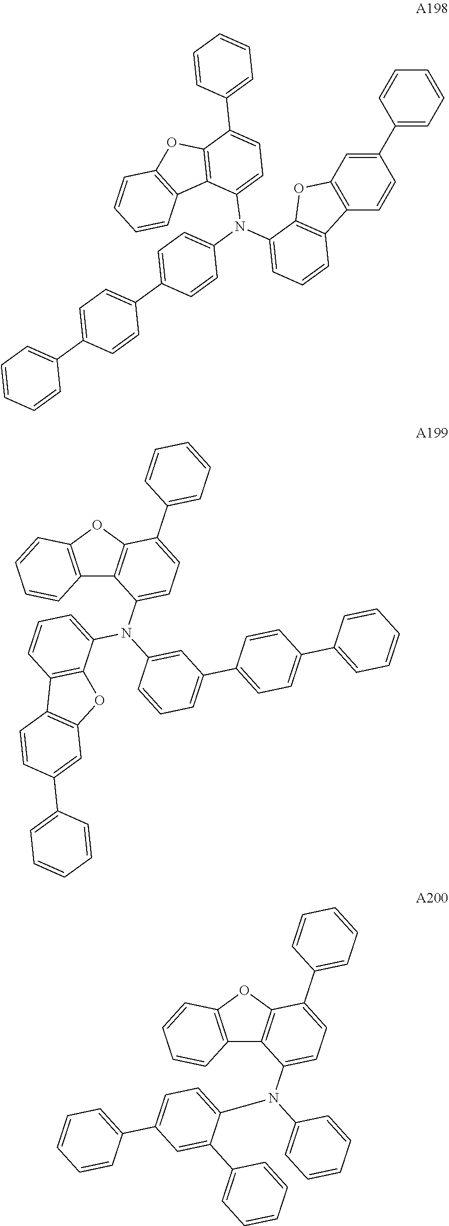 [SMILES: c1ccc(-c2ccc(-c3ccc(N(c4cccc5c4oc4cc(-c6ccccc6)ccc45)c4ccc(-c5ccccc5)c5oc6ccccc6c45)cc3)cc2)cc1 ; c1ccc(-c2ccc(-c3cccc(N(c4cccc5c4oc4cc(-c6ccccc6)ccc45)c4ccc(-c5ccccc5)c5oc6ccccc6c45)c3)cc2)cc1 ; c1ccc(-c2ccc(N(c3ccccc3)c3ccc(-c4ccccc4)c4oc5ccccc5c34)c(-c3ccccc3)c2)cc1]

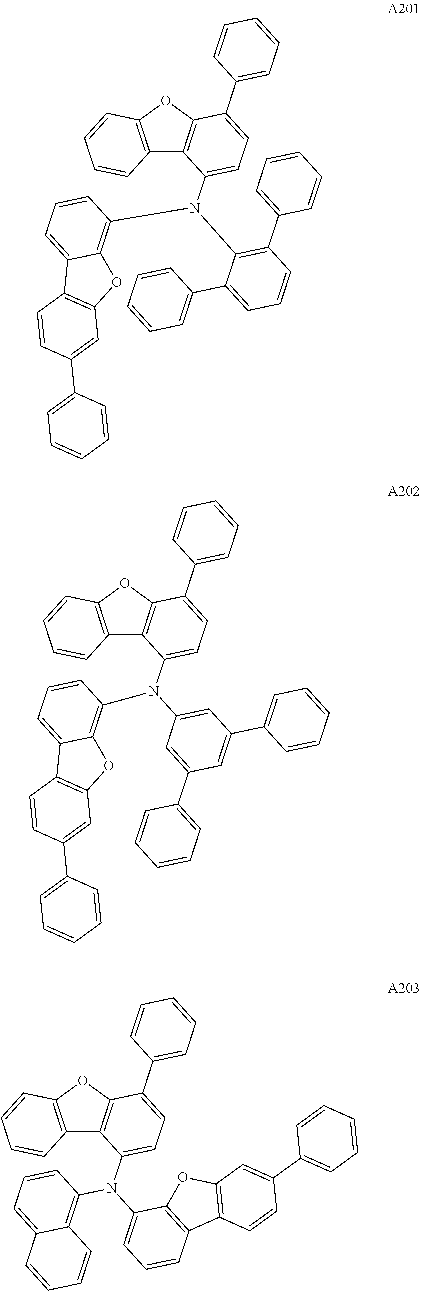 [SMILES: c1ccc(-c2ccc3c(c2)oc2c(N(c4c(-c5ccccc5)cccc4-c4ccccc4)c4ccc(-c5ccccc5)c5oc6ccccc6c45)cccc23)cc1 ; c1ccc(-c2cc(-c3ccccc3)cc(N(c3cccc4c3oc3cc(-c5ccccc5)ccc34)c3ccc(-c4ccccc4)c4oc5ccccc5c34)c2)cc1 ; c1ccc(-c2ccc3c(c2)oc2c(N(c4cccc5ccccc45)c4ccc(-c5ccccc5)c5oc6ccccc6c45)cccc23)cc1]

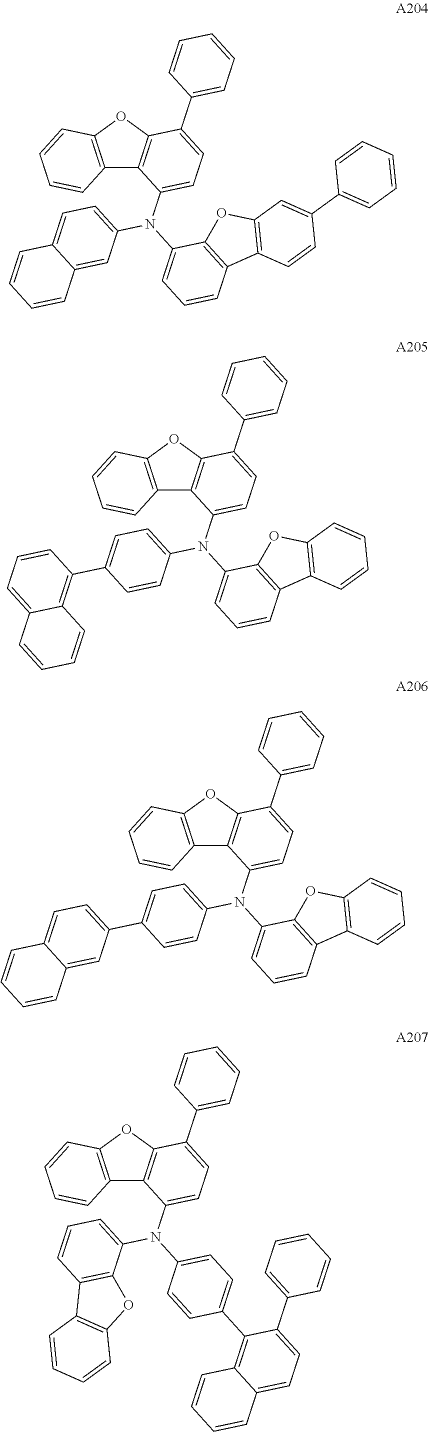 [SMILES: c1ccc(-c2ccc3c(c2)oc2c(N(c4ccc5ccccc5c4)c4ccc(-c5ccccc5)c5oc6ccccc6c45)cccc23)cc1 ; c1ccc(-c2ccc(N(c3ccc(-c4cccc5ccccc45)cc3)c3cccc4c3oc3ccccc34)c3c2oc2ccccc23)cc1 ; c1ccc(-c2ccc(N(c3ccc(-c4ccc5ccccc5c4)cc3)c3cccc4c3oc3ccccc34)c3c2oc2ccccc23)cc1 ; c1ccc(-c2ccc3ccccc3c2-c2ccc(N(c3cccc4c3oc3ccccc34)c3ccc(-c4ccccc4)c4oc5ccccc5c34)cc2)cc1]

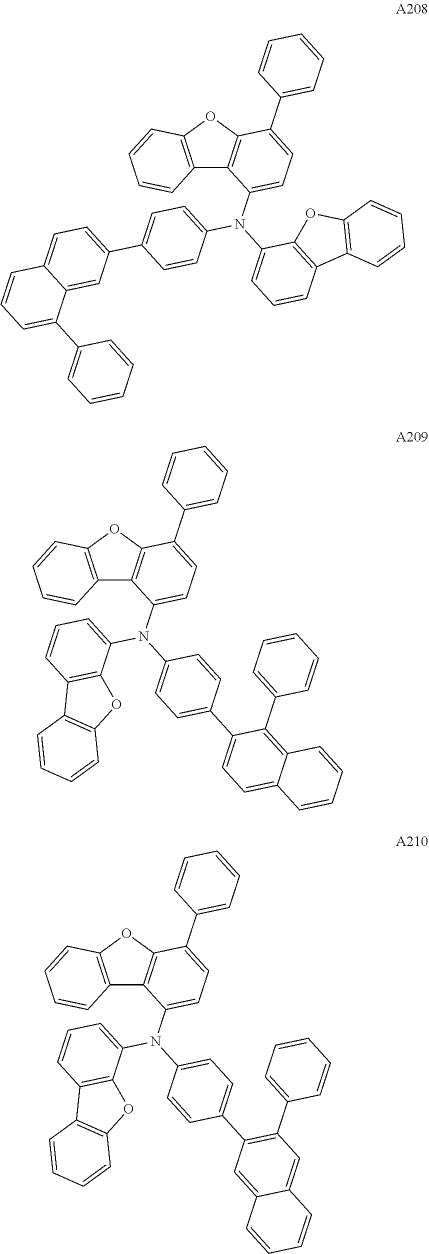 [SMILES: c1ccc(-c2cccc3ccc(-c4ccc(N(c5cccc6c5oc5ccccc56)c5ccc(-c6ccccc6)c6oc7ccccc7c56)cc4)cc23)cc1 ; c1ccc(-c2c(-c3ccc(N(c4cccc5c4oc4ccccc45)c4ccc(-c5ccccc5)c5oc6ccccc6c45)cc3)ccc3ccccc23)cc1 ; c1ccc(-c2cc3ccccc3cc2-c2ccc(N(c3cccc4c3oc3ccccc34)c3ccc(-c4ccccc4)c4oc5ccccc5c34)cc2)cc1]

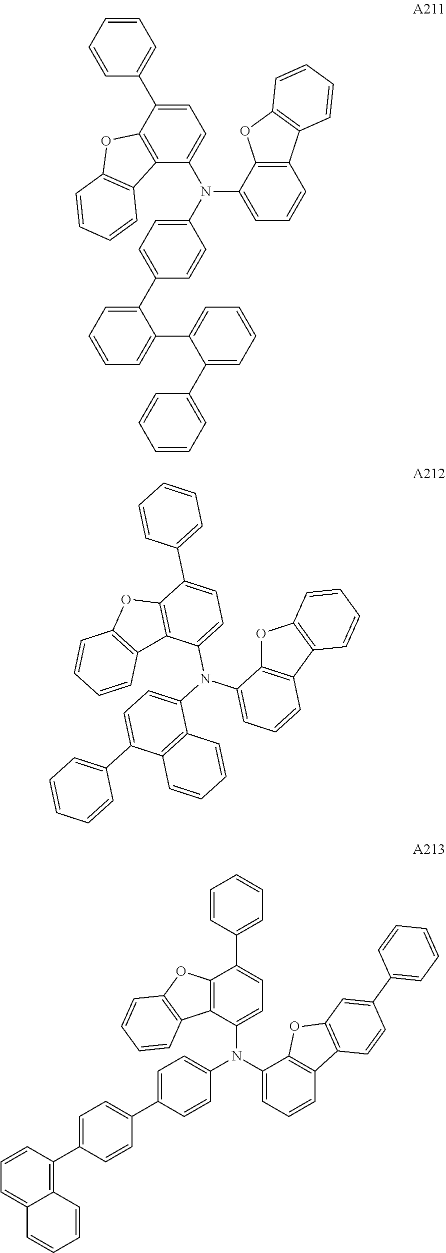

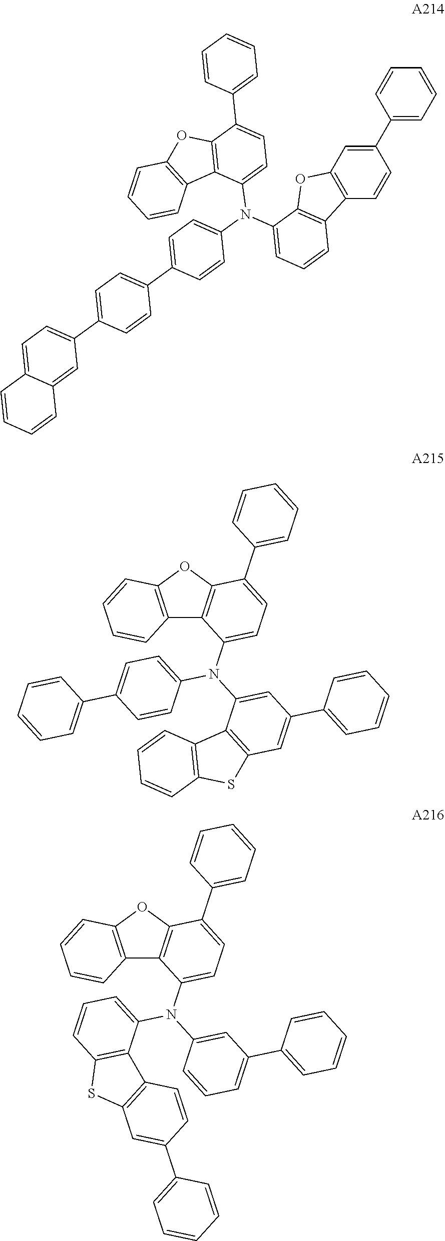

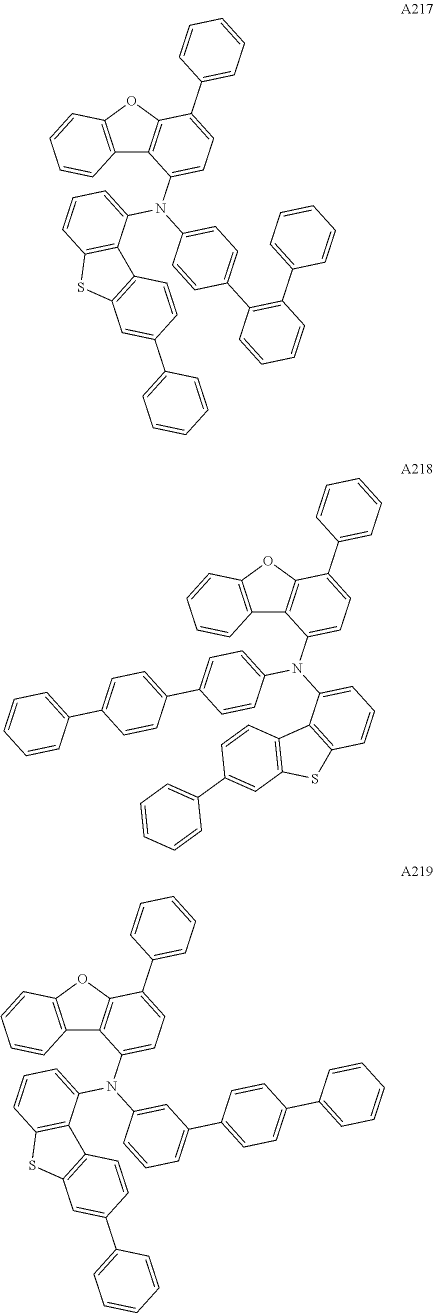

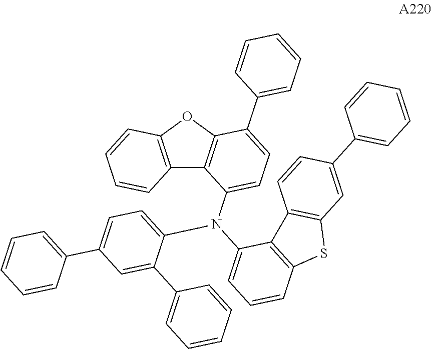 [SMILES: c1ccc(-c2ccc(N(c3cccc4sc5cc(-c6ccccc6)ccc5c34)c3ccc(-c4ccccc4)c4oc5ccccc5c34)c(-c3ccccc3)c2)cc1]

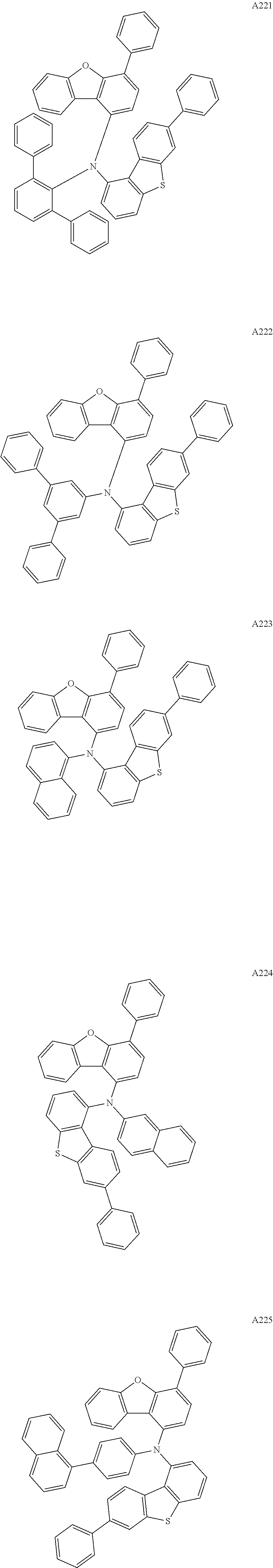 [SMILES: c1ccc(-c2ccc3c(c2)sc2cccc(N(c4c(-c5ccccc5)cccc4-c4ccccc4)c4ccc(-c5ccccc5)c5oc6ccccc6c45)c23)cc1 ; c1ccc(-c2cc(-c3ccccc3)cc(N(c3cccc4sc5cc(-c6ccccc6)ccc5c34)c3ccc(-c4ccccc4)c4oc5ccccc5c34)c2)cc1 ; c1ccc(-c2ccc3c(c2)sc2cccc(N(c4cccc5ccccc45)c4ccc(-c5ccccc5)c5oc6ccccc6c45)c23)cc1 ; c1ccc(-c2ccc3c(c2)sc2cccc(N(c4ccc5ccccc5c4)c4ccc(-c5ccccc5)c5oc6ccccc6c45)c23)cc1 ; c1ccc(-c2ccc3c(c2)sc2cccc(N(c4ccc(-c5cccc6ccccc56)cc4)c4ccc(-c5ccccc5)c5oc6ccccc6c45)c23)cc1]

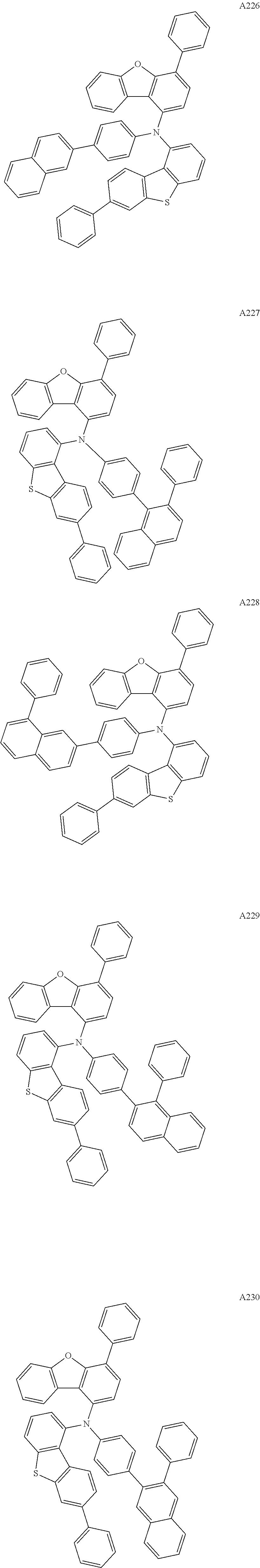

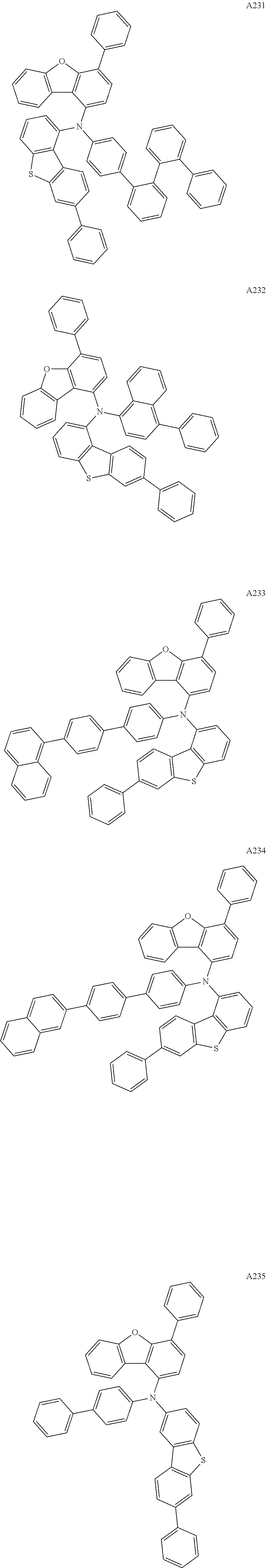 [SMILES: c1ccc(-c2ccc3c(c2)sc2cccc(N(c4ccc(-c5ccccc5-c5ccccc5-c5ccccc5)cc4)c4ccc(-c5ccccc5)c5oc6ccccc6c45)c23)cc1 ; c1ccc(-c2ccc3c(c2)sc2cccc(N(c4ccc(-c5ccccc5)c5ccccc45)c4ccc(-c5ccccc5)c5oc6ccccc6c45)c23)cc1 ; c1ccc(-c2ccc3c(c2)sc2cccc(N(c4ccc(-c5ccc(-c6cccc7ccccc67)cc5)cc4)c4ccc(-c5ccccc5)c5oc6ccccc6c45)c23)cc1 ; c1ccc(-c2ccc3c(c2)sc2cccc(N(c4ccc(-c5ccc(-c6ccc7ccccc7c6)cc5)cc4)c4ccc(-c5ccccc5)c5oc6ccccc6c45)c23)cc1 ; c1ccc(-c2ccc(N(c3ccc4sc5cc(-c6ccccc6)ccc5c4c3)c3ccc(-c4ccccc4)c4oc5ccccc5c34)cc2)cc1]

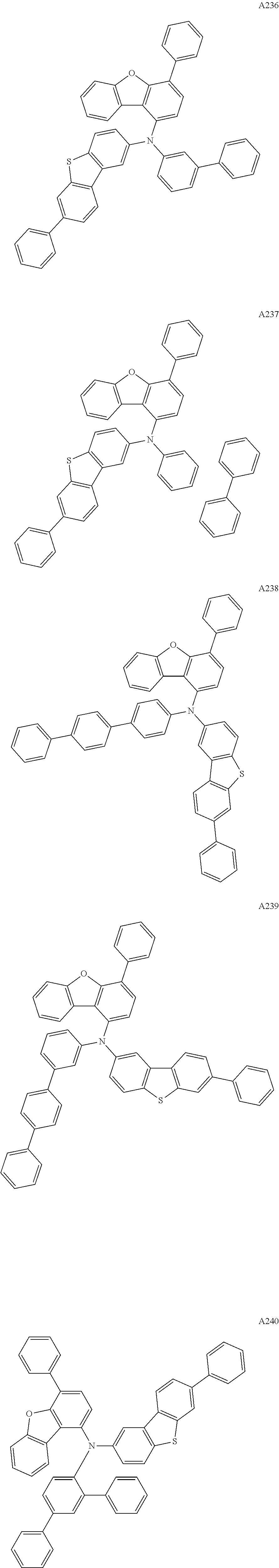

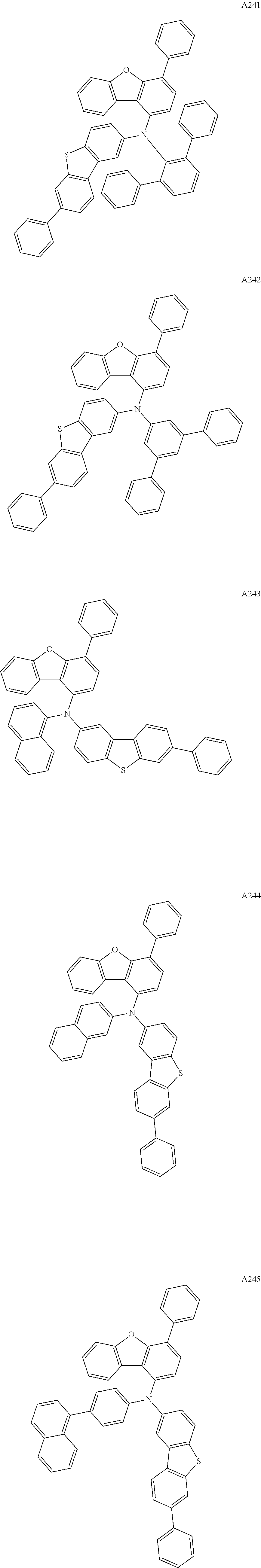 [SMILES: c1ccc(-c2ccc3c(c2)sc2ccc(N(c4c(-c5ccccc5)cccc4-c4ccccc4)c4ccc(-c5ccccc5)c5oc6ccccc6c45)cc23)cc1 ; c1ccc(-c2cc(-c3ccccc3)cc(N(c3ccc4sc5cc(-c6ccccc6)ccc5c4c3)c3ccc(-c4ccccc4)c4oc5ccccc5c34)c2)cc1 ; c1ccc(-c2ccc3c(c2)sc2ccc(N(c4cccc5ccccc45)c4ccc(-c5ccccc5)c5oc6ccccc6c45)cc23)cc1 ; c1ccc(-c2ccc3c(c2)sc2ccc(N(c4ccc5ccccc5c4)c4ccc(-c5ccccc5)c5oc6ccccc6c45)cc23)cc1 ; c1ccc(-c2ccc3c(c2)sc2ccc(N(c4ccc(-c5cccc6ccccc56)cc4)c4ccc(-c5ccccc5)c5oc6ccccc6c45)cc23)cc1]

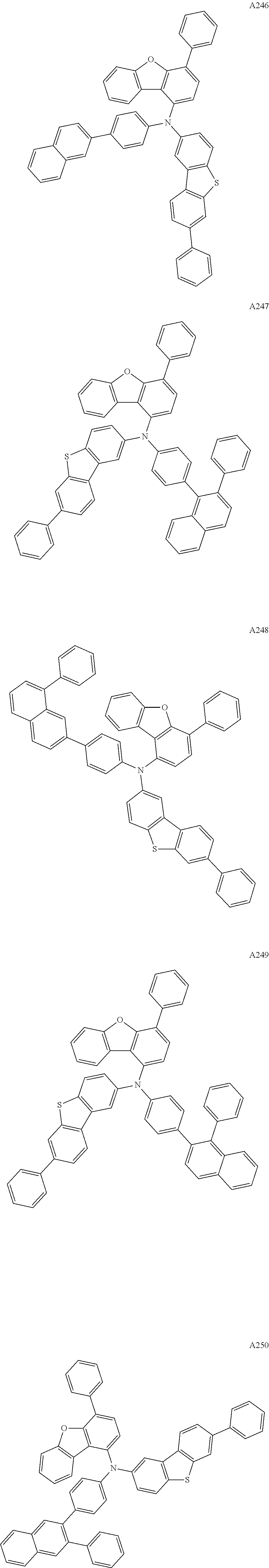

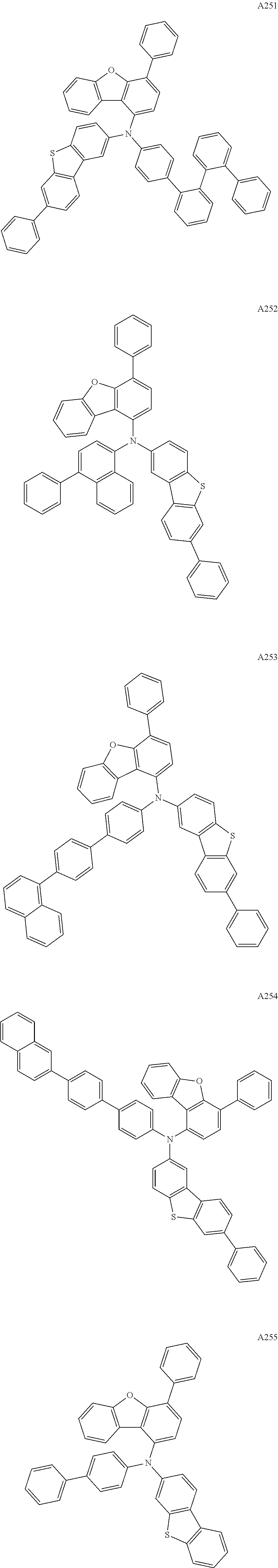 [SMILES: c1ccc(-c2ccc3c(c2)sc2ccc(N(c4ccc(-c5ccccc5-c5ccccc5-c5ccccc5)cc4)c4ccc(-c5ccccc5)c5oc6ccccc6c45)cc23)cc1 ; c1ccc(-c2ccc3c(c2)sc2ccc(N(c4ccc(-c5ccccc5)c5ccccc45)c4ccc(-c5ccccc5)c5oc6ccccc6c45)cc23)cc1 ; c1ccc(-c2ccc3c(c2)sc2ccc(N(c4ccc(-c5ccc(-c6cccc7ccccc67)cc5)cc4)c4ccc(-c5ccccc5)c5oc6ccccc6c45)cc23)cc1 ; c1ccc(-c2ccc3c(c2)sc2ccc(N(c4ccc(-c5ccc(-c6ccc7ccccc7c6)cc5)cc4)c4ccc(-c5ccccc5)c5oc6ccccc6c45)cc23)cc1 ; c1ccc(-c2ccc(N(c3ccc4c(c3)sc3ccccc34)c3ccc(-c4ccccc4)c4oc5ccccc5c34)cc2)cc1]

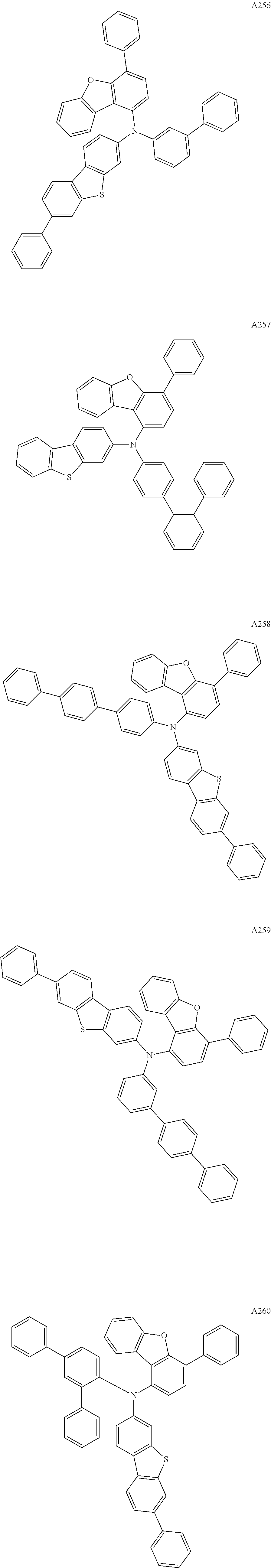 [SMILES: c1ccc(-c2cccc(N(c3ccc4c(c3)sc3cc(-c5ccccc5)ccc34)c3ccc(-c4ccccc4)c4oc5ccccc5c34)c2)cc1 ; c1ccc(-c2ccccc2-c2ccc(N(c3ccc4c(c3)sc3ccccc34)c3ccc(-c4ccccc4)c4oc5ccccc5c34)cc2)cc1 ; c1ccc(-c2ccc(-c3ccc(N(c4ccc5c(c4)sc4cc(-c6ccccc6)ccc45)c4ccc(-c5ccccc5)c5oc6ccccc6c45)cc3)cc2)cc1 ; c1ccc(-c2ccc(-c3cccc(N(c4ccc5c(c4)sc4cc(-c6ccccc6)ccc45)c4ccc(-c5ccccc5)c5oc6ccccc6c45)c3)cc2)cc1 ; c1ccc(-c2ccc(N(c3ccc4c(c3)sc3cc(-c5ccccc5)ccc34)c3ccc(-c4ccccc4)c4oc5ccccc5c34)c(-c3ccccc3)c2)cc1]

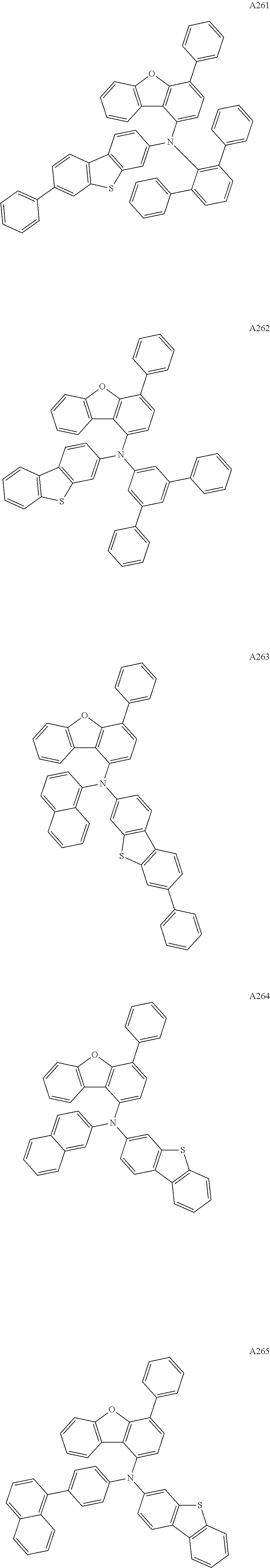 [SMILES: c1ccc(-c2ccc3c(c2)sc2cc(N(c4c(-c5ccccc5)cccc4-c4ccccc4)c4ccc(-c5ccccc5)c5oc6ccccc6c45)ccc23)cc1 ; c1ccc(-c2cc(-c3ccccc3)cc(N(c3ccc4c(c3)sc3ccccc34)c3ccc(-c4ccccc4)c4oc5ccccc5c34)c2)cc1 ; c1ccc(-c2ccc3c(c2)sc2cc(N(c4cccc5ccccc45)c4ccc(-c5ccccc5)c5oc6ccccc6c45)ccc23)cc1 ; c1ccc(-c2ccc(N(c3ccc4ccccc4c3)c3ccc4c(c3)sc3ccccc34)c3c2oc2ccccc23)cc1 ; c1ccc(-c2ccc(N(c3ccc(-c4cccc5ccccc45)cc3)c3ccc4c(c3)sc3ccccc34)c3c2oc2ccccc23)cc1]

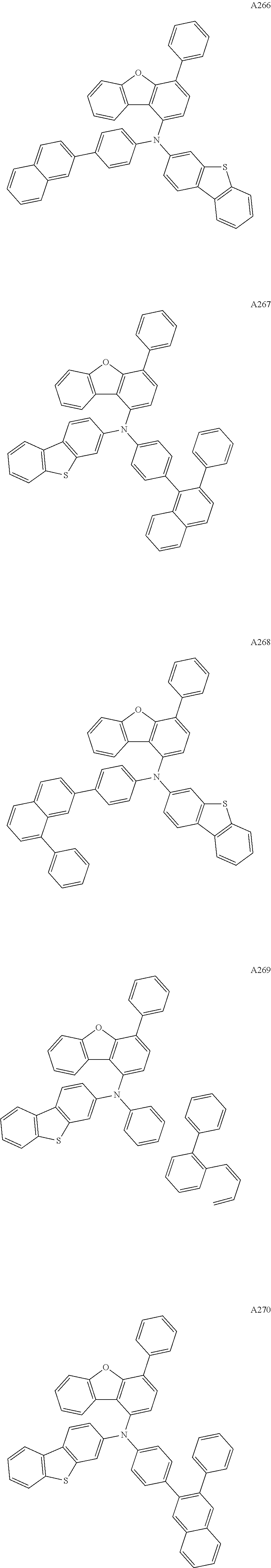

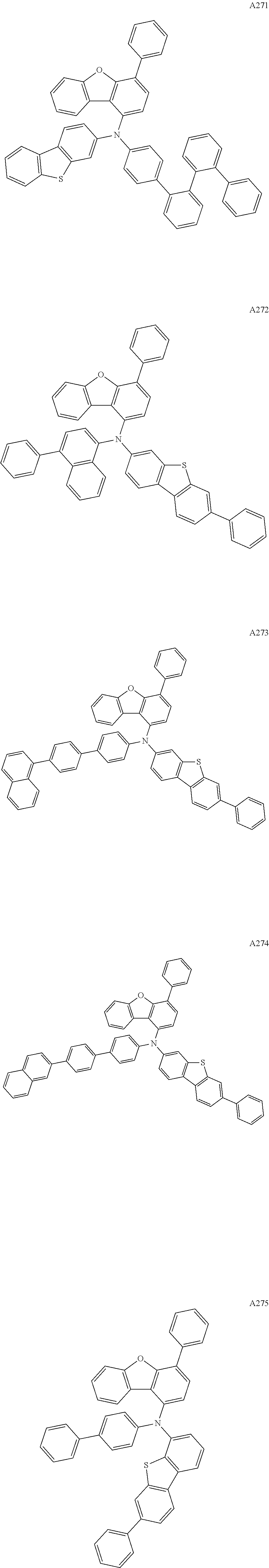 [SMILES: c1ccc(-c2ccccc2-c2ccccc2-c2ccc(N(c3ccc4c(c3)sc3ccccc34)c3ccc(-c4ccccc4)c4oc5ccccc5c34)cc2)cc1 ; c1ccc(-c2ccc3c(c2)sc2cc(N(c4ccc(-c5ccccc5)c5ccccc45)c4ccc(-c5ccccc5)c5oc6ccccc6c45)ccc23)cc1 ; c1ccc(-c2ccc3c(c2)sc2cc(N(c4ccc(-c5ccc(-c6cccc7ccccc67)cc5)cc4)c4ccc(-c5ccccc5)c5oc6ccccc6c45)ccc23)cc1 ; c1ccc(-c2ccc3c(c2)sc2cc(N(c4ccc(-c5ccc(-c6ccc7ccccc7c6)cc5)cc4)c4ccc(-c5ccccc5)c5oc6ccccc6c45)ccc23)cc1 ; c1ccc(-c2ccc(N(c3cccc4c3sc3cc(-c5ccccc5)ccc34)c3ccc(-c4ccccc4)c4oc5ccccc5c34)cc2)cc1]

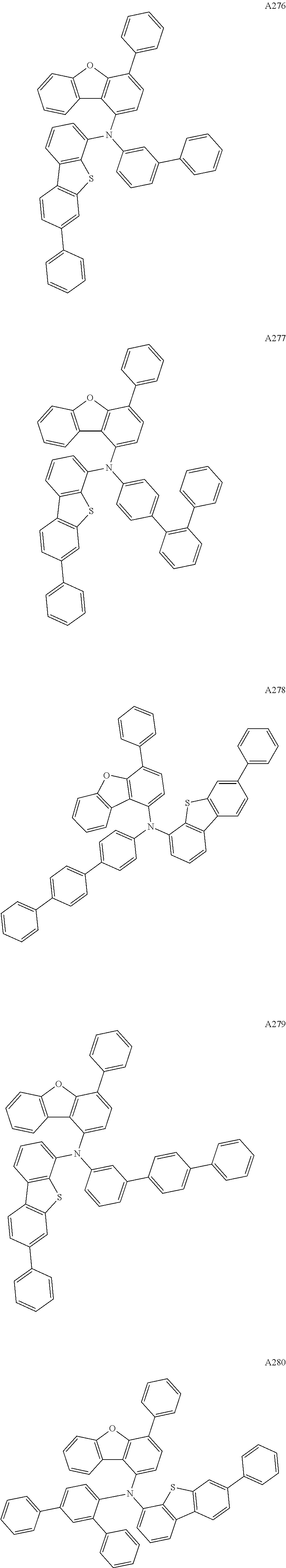 [SMILES: c1ccc(-c2cccc(N(c3cccc4c3sc3cc(-c5ccccc5)ccc34)c3ccc(-c4ccccc4)c4oc5ccccc5c34)c2)cc1 ; c1ccc(-c2ccc3c(c2)sc2c(N(c4ccc(-c5ccccc5-c5ccccc5)cc4)c4ccc(-c5ccccc5)c5oc6ccccc6c45)cccc23)cc1 ; c1ccc(-c2ccc(-c3ccc(N(c4cccc5c4sc4cc(-c6ccccc6)ccc45)c4ccc(-c5ccccc5)c5oc6ccccc6c45)cc3)cc2)cc1 ; c1ccc(-c2ccc(-c3cccc(N(c4cccc5c4sc4cc(-c6ccccc6)ccc45)c4ccc(-c5ccccc5)c5oc6ccccc6c45)c3)cc2)cc1 ; c1ccc(-c2ccc(N(c3cccc4c3sc3cc(-c5ccccc5)ccc34)c3ccc(-c4ccccc4)c4oc5ccccc5c34)c(-c3ccccc3)c2)cc1]

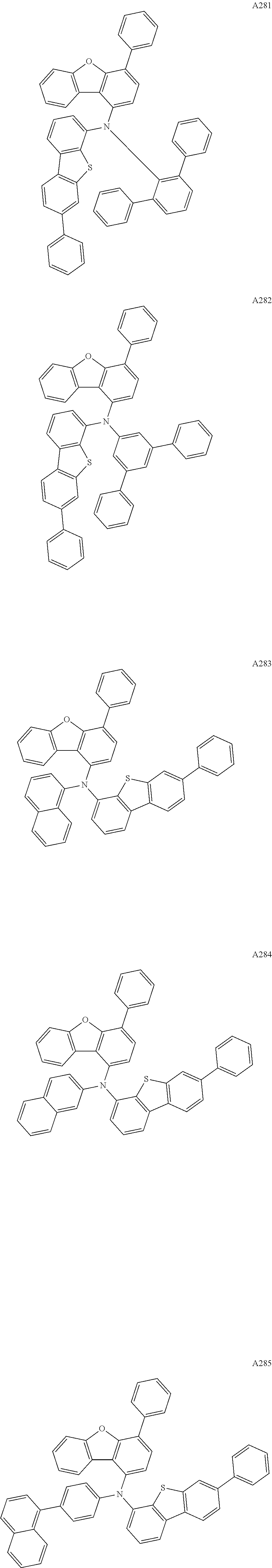 [SMILES: c1ccc(-c2ccc3c(c2)sc2c(N(c4c(-c5ccccc5)cccc4-c4ccccc4)c4ccc(-c5ccccc5)c5oc6ccccc6c45)cccc23)cc1 ; c1ccc(-c2cc(-c3ccccc3)cc(N(c3cccc4c3sc3cc(-c5ccccc5)ccc34)c3ccc(-c4ccccc4)c4oc5ccccc5c34)c2)cc1 ; c1ccc(-c2ccc3c(c2)sc2c(N(c4cccc5ccccc45)c4ccc(-c5ccccc5)c5oc6ccccc6c45)cccc23)cc1 ; c1ccc(-c2ccc3c(c2)sc2c(N(c4ccc5ccccc5c4)c4ccc(-c5ccccc5)c5oc6ccccc6c45)cccc23)cc1 ; c1ccc(-c2ccc3c(c2)sc2c(N(c4ccc(-c5cccc6ccccc56)cc4)c4ccc(-c5ccccc5)c5oc6ccccc6c45)cccc23)cc1]

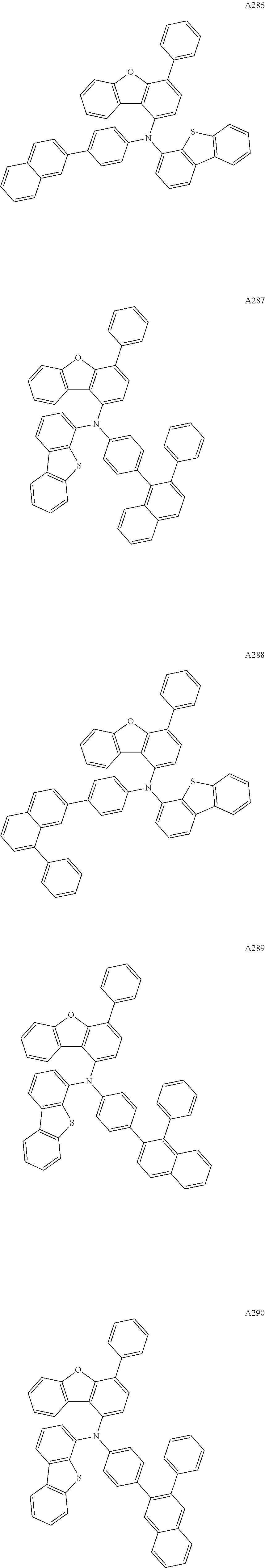 [SMILES: c1ccc(-c2ccc(N(c3ccc(-c4ccc5ccccc5c4)cc3)c3cccc4c3sc3ccccc34)c3c2oc2ccccc23)cc1 ; c1ccc(-c2ccc3ccccc3c2-c2ccc(N(c3cccc4c3sc3ccccc34)c3ccc(-c4ccccc4)c4oc5ccccc5c34)cc2)cc1 ; c1ccc(-c2cccc3ccc(-c4ccc(N(c5cccc6c5sc5ccccc56)c5ccc(-c6ccccc6)c6oc7ccccc7c56)cc4)cc23)cc1 ; c1ccc(-c2c(-c3ccc(N(c4cccc5c4sc4ccccc45)c4ccc(-c5ccccc5)c5oc6ccccc6c45)cc3)ccc3ccccc23)cc1 ; c1ccc(-c2cc3ccccc3cc2-c2ccc(N(c3cccc4c3sc3ccccc34)c3ccc(-c4ccccc4)c4oc5ccccc5c34)cc2)cc1]

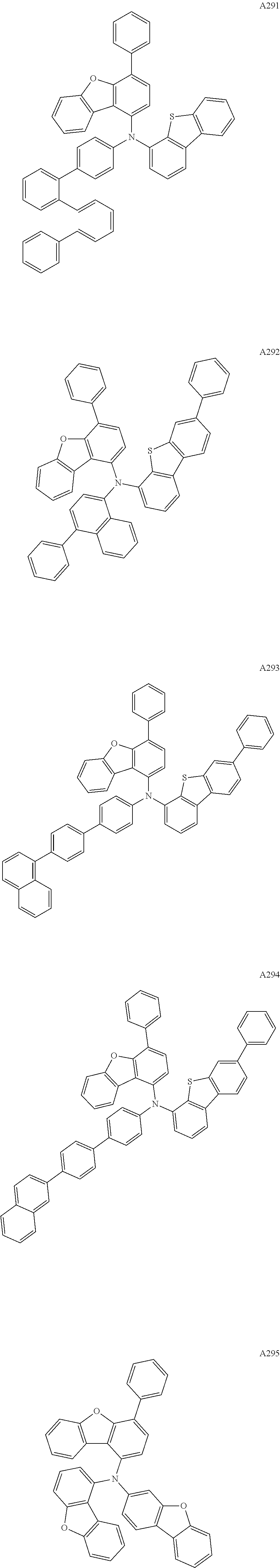 [SMILES: C(=C/C=C/c1ccccc1-c1ccc(N(c2cccc3c2sc2ccccc23)c2ccc(-c3ccccc3)c3oc4ccccc4c23)cc1)/C=C/c1ccccc1 ; c1ccc(-c2ccc3c(c2)sc2c(N(c4ccc(-c5ccccc5)c5ccccc45)c4ccc(-c5ccccc5)c5oc6ccccc6c45)cccc23)cc1 ; c1ccc(-c2ccc3c(c2)sc2c(N(c4ccc(-c5ccc(-c6cccc7ccccc67)cc5)cc4)c4ccc(-c5ccccc5)c5oc6ccccc6c45)cccc23)cc1 ; c1ccc(-c2ccc3c(c2)sc2c(N(c4ccc(-c5ccc(-c6ccc7ccccc7c6)cc5)cc4)c4ccc(-c5ccccc5)c5oc6ccccc6c45)cccc23)cc1 ; c1ccc(-c2ccc(N(c3ccc4c(c3)oc3ccccc34)c3cccc4oc5ccccc5c34)c3c2oc2ccccc23)cc1]

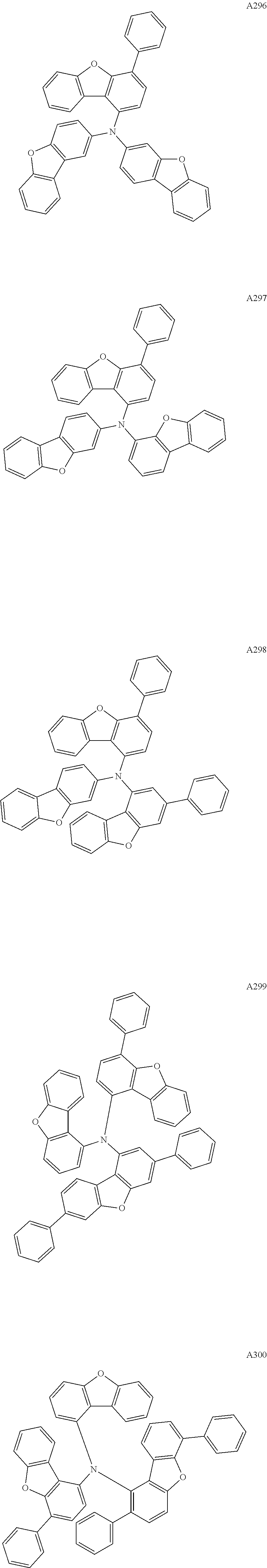 [SMILES: c1ccc(-c2ccc(N(c3ccc4c(c3)oc3ccccc34)c3ccc4oc5ccccc5c4c3)c3c2oc2ccccc23)cc1 ; c1ccc(-c2ccc(N(c3ccc4c(c3)oc3ccccc34)c3cccc4c3oc3ccccc34)c3c2oc2ccccc23)cc1 ; c1ccc(-c2cc(N(c3ccc4c(c3)oc3ccccc34)c3ccc(-c4ccccc4)c4oc5ccccc5c34)c3c(c2)oc2ccccc23)cc1 ; c1ccc(-c2ccc3c(c2)oc2cc(-c4ccccc4)cc(N(c4cccc5oc6ccccc6c45)c4ccc(-c5ccccc5)c5oc6ccccc6c45)c23)cc1 ; c1ccc(-c2ccc3oc4c(-c5ccccc5)cccc4c3c2N(c2cccc3oc4ccccc4c23)c2ccc(-c3ccccc3)c3oc4ccccc4c23)cc1]

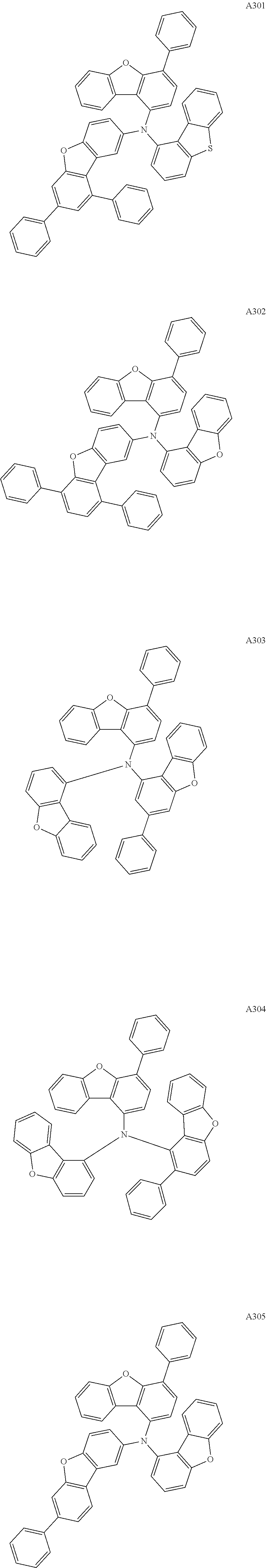 [SMILES: c1ccc(-c2cc(-c3ccccc3)c3c(c2)oc2ccc(N(c4cccc5sc6ccccc6c45)c4ccc(-c5ccccc5)c5oc6ccccc6c45)cc23)cc1 ; c1ccc(-c2ccc(-c3ccccc3)c3c2oc2ccc(N(c4cccc5oc6ccccc6c45)c4ccc(-c5ccccc5)c5oc6ccccc6c45)cc23)cc1 ; c1ccc(-c2cc(N(c3cccc4oc5ccccc5c34)c3ccc(-c4ccccc4)c4oc5ccccc5c34)c3c(c2)oc2ccccc23)cc1 ; c1ccc(-c2ccc3oc4ccccc4c3c2N(c2cccc3oc4ccccc4c23)c2ccc(-c3ccccc3)c3oc4ccccc4c23)cc1 ; c1ccc(-c2ccc3c(c2)oc2ccc(N(c4cccc5oc6ccccc6c45)c4ccc(-c5ccccc5)c5oc6ccccc6c45)cc23)cc1]

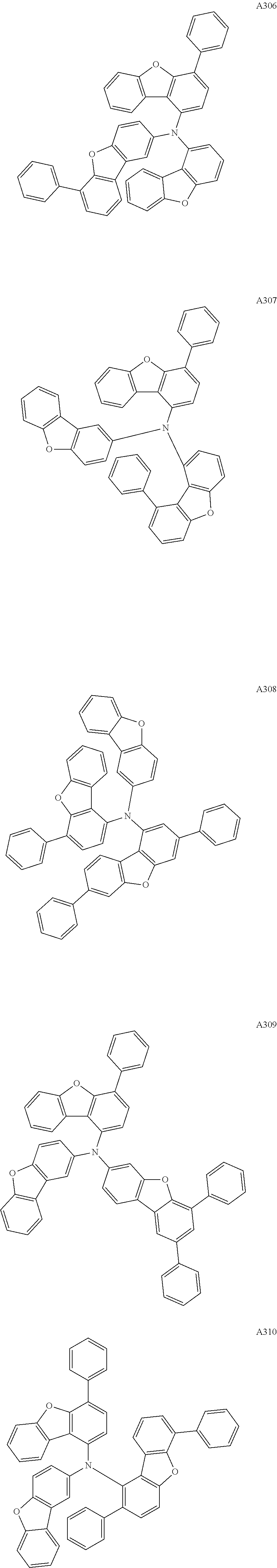 [SMILES: c1ccc(-c2cccc3c2oc2ccc(N(c4cccc5oc6ccccc6c45)c4ccc(-c5ccccc5)c5oc6ccccc6c45)cc23)cc1 ; c1ccc(-c2ccc(N(c3ccc4oc5ccccc5c4c3)c3cccc4oc5cccc(-c6ccccc6)c5c34)c3c2oc2ccccc23)cc1 ; c1ccc(-c2ccc3c(c2)oc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3oc4cc(N(c5ccc6oc7ccccc7c6c5)c5ccc(-c6ccccc6)c6oc7ccccc7c56)ccc4c3c2)cc1 ; c1ccc(-c2ccc3oc4c(-c5ccccc5)cccc4c3c2N(c2ccc3oc4ccccc4c3c2)c2ccc(-c3ccccc3)c3oc4ccccc4c23)cc1]

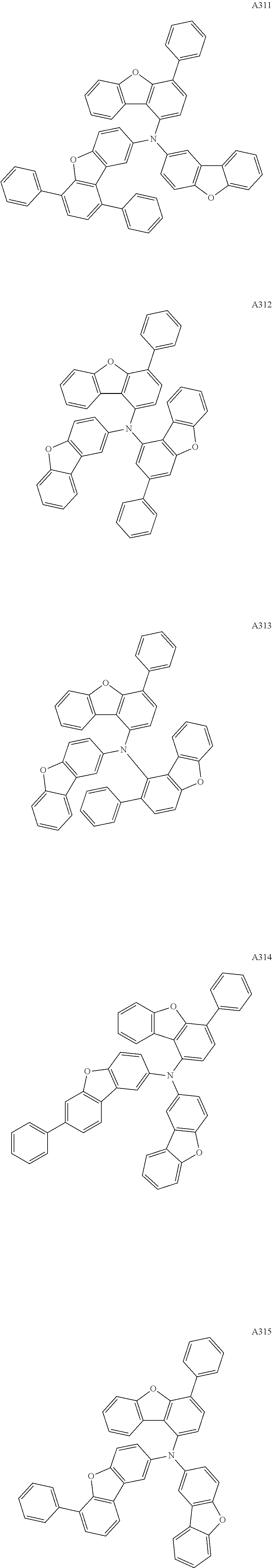 [SMILES: c1ccc(-c2ccc(-c3ccccc3)c3c2oc2ccc(N(c4ccc5oc6ccccc6c5c4)c4ccc(-c5ccccc5)c5oc6ccccc6c45)cc23)cc1 ; c1ccc(-c2cc(N(c3ccc4oc5ccccc5c4c3)c3ccc(-c4ccccc4)c4oc5ccccc5c34)c3c(c2)oc2ccccc23)cc1 ; c1ccc(-c2ccc3oc4ccccc4c3c2N(c2ccc3oc4ccccc4c3c2)c2ccc(-c3ccccc3)c3oc4ccccc4c23)cc1 ; c1ccc(-c2ccc3c(c2)oc2ccc(N(c4ccc5oc6ccccc6c5c4)c4ccc(-c5ccccc5)c5oc6ccccc6c45)cc23)cc1 ; c1ccc(-c2cccc3c2oc2ccc(N(c4ccc5oc6ccccc6c5c4)c4ccc(-c5ccccc5)c5oc6ccccc6c45)cc23)cc1]

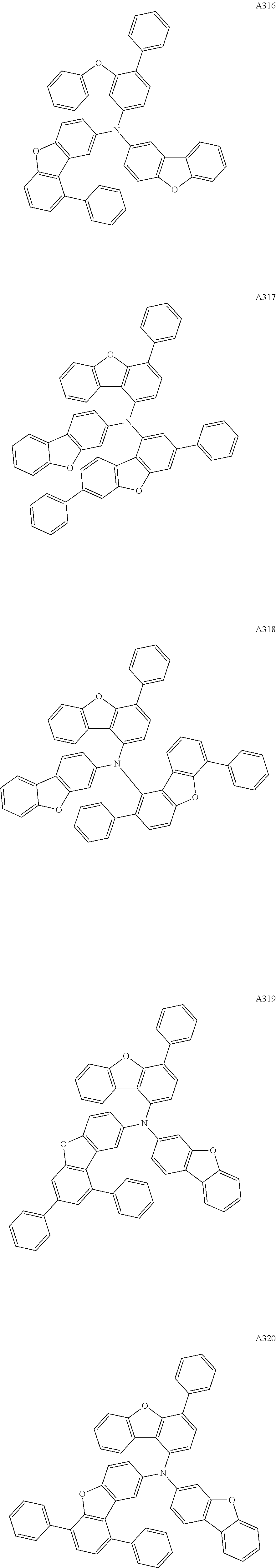 [SMILES: c1ccc(-c2ccc(N(c3ccc4oc5ccccc5c4c3)c3ccc4oc5cccc(-c6ccccc6)c5c4c3)c3c2oc2ccccc23)cc1 ; c1ccc(-c2ccc3c(c2)oc2cc(-c4ccccc4)cc(N(c4ccc5c(c4)oc4ccccc45)c4ccc(-c5ccccc5)c5oc6ccccc6c45)c23)cc1 ; c1ccc(-c2ccc3oc4c(-c5ccccc5)cccc4c3c2N(c2ccc3c(c2)oc2ccccc23)c2ccc(-c3ccccc3)c3oc4ccccc4c23)cc1 ; c1ccc(-c2cc(-c3ccccc3)c3c(c2)oc2ccc(N(c4ccc5c(c4)oc4ccccc45)c4ccc(-c5ccccc5)c5oc6ccccc6c45)cc23)cc1 ; c1ccc(-c2ccc(-c3ccccc3)c3c2oc2ccc(N(c4ccc5c(c4)oc4ccccc45)c4ccc(-c5ccccc5)c5oc6ccccc6c45)cc23)cc1]

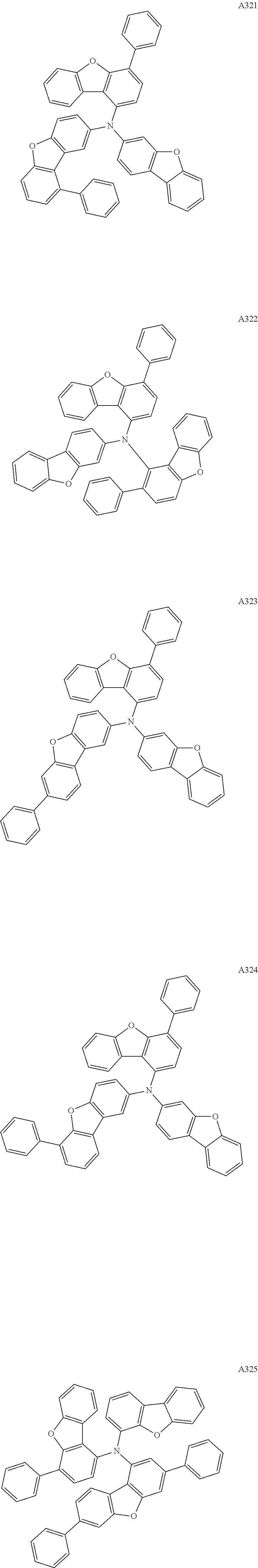 [SMILES: c1ccc(-c2ccc(N(c3ccc4c(c3)oc3ccccc34)c3ccc4oc5cccc(-c6ccccc6)c5c4c3)c3c2oc2ccccc23)cc1 ; c1ccc(-c2ccc3oc4ccccc4c3c2N(c2ccc3c(c2)oc2ccccc23)c2ccc(-c3ccccc3)c3oc4ccccc4c23)cc1 ; c1ccc(-c2ccc3c(c2)oc2ccc(N(c4ccc5c(c4)oc4ccccc45)c4ccc(-c5ccccc5)c5oc6ccccc6c45)cc23)cc1 ; c1ccc(-c2cccc3c2oc2ccc(N(c4ccc5c(c4)oc4ccccc45)c4ccc(-c5ccccc5)c5oc6ccccc6c45)cc23)cc1 ; c1ccc(-c2ccc3c(c2)oc2cc(-c4ccccc4)cc(N(c4cccc5c4oc4ccccc45)c4ccc(-c5ccccc5)c5oc6ccccc6c45)c23)cc1]

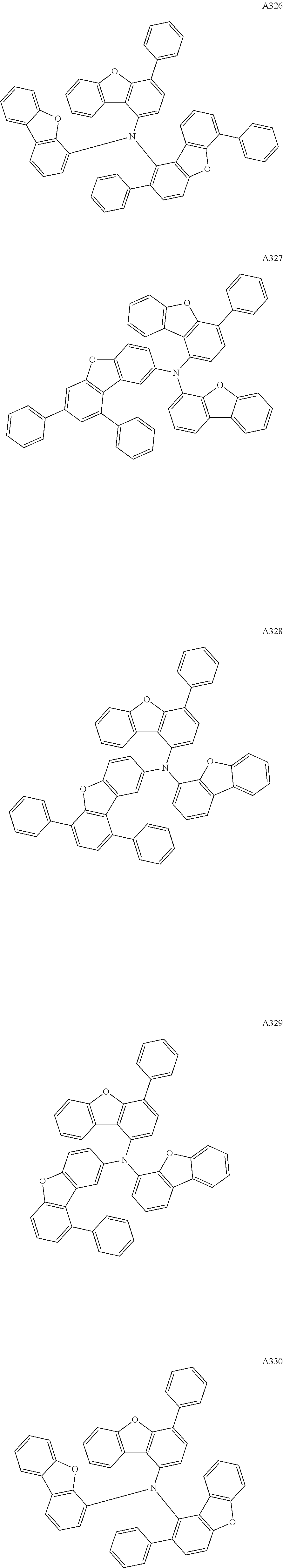 [SMILES: c1ccc(-c2ccc3oc4c(-c5ccccc5)cccc4c3c2N(c2cccc3c2oc2ccccc23)c2ccc(-c3ccccc3)c3oc4ccccc4c23)cc1 ; c1ccc(-c2cc(-c3ccccc3)c3c(c2)oc2ccc(N(c4cccc5c4oc4ccccc45)c4ccc(-c5ccccc5)c5oc6ccccc6c45)cc23)cc1 ; c1ccc(-c2ccc(-c3ccccc3)c3c2oc2ccc(N(c4cccc5c4oc4ccccc45)c4ccc(-c5ccccc5)c5oc6ccccc6c45)cc23)cc1 ; c1ccc(-c2ccc(N(c3ccc4oc5cccc(-c6ccccc6)c5c4c3)c3cccc4c3oc3ccccc34)c3c2oc2ccccc23)cc1 ; c1ccc(-c2ccc3oc4ccccc4c3c2N(c2cccc3c2oc2ccccc23)c2ccc(-c3ccccc3)c3oc4ccccc4c23)cc1]

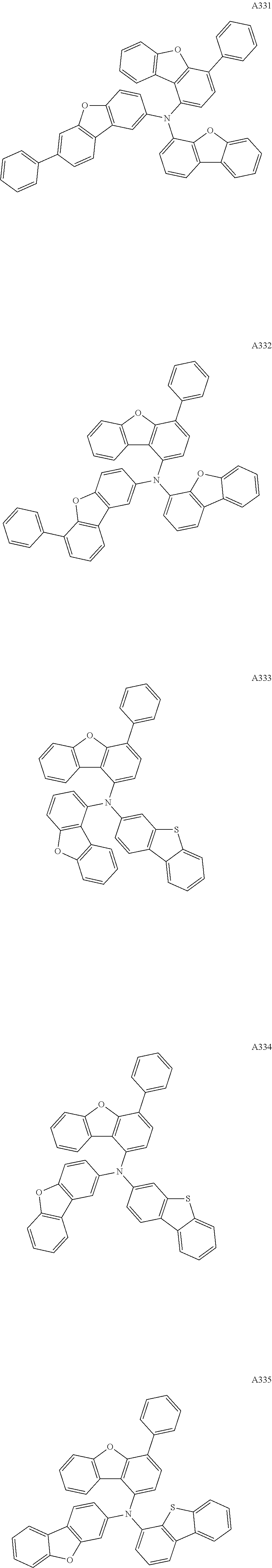 [SMILES: c1ccc(-c2ccc3c(c2)oc2ccc(N(c4cccc5c4oc4ccccc45)c4ccc(-c5ccccc5)c5oc6ccccc6c45)cc23)cc1 ; c1ccc(-c2cccc3c2oc2ccc(N(c4cccc5c4oc4ccccc45)c4ccc(-c5ccccc5)c5oc6ccccc6c45)cc23)cc1 ; c1ccc(-c2ccc(N(c3ccc4c(c3)sc3ccccc34)c3cccc4oc5ccccc5c34)c3c2oc2ccccc23)cc1 ; c1ccc(-c2ccc(N(c3ccc4c(c3)sc3ccccc34)c3ccc4oc5ccccc5c4c3)c3c2oc2ccccc23)cc1 ; c1ccc(-c2ccc(N(c3ccc4c(c3)oc3ccccc34)c3cccc4c3sc3ccccc34)c3c2oc2ccccc23)cc1]

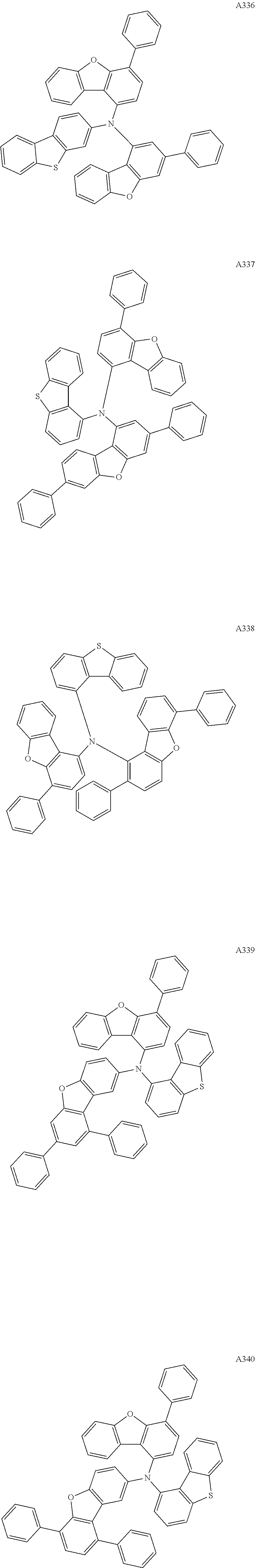 [SMILES: c1ccc(-c2cc(N(c3ccc4c(c3)sc3ccccc34)c3ccc(-c4ccccc4)c4oc5ccccc5c34)c3c(c2)oc2ccccc23)cc1 ; c1ccc(-c2ccc3c(c2)oc2cc(-c4ccccc4)cc(N(c4cccc5sc6ccccc6c45)c4ccc(-c5ccccc5)c5oc6ccccc6c45)c23)cc1 ; c1ccc(-c2ccc3oc4c(-c5ccccc5)cccc4c3c2N(c2cccc3sc4ccccc4c23)c2ccc(-c3ccccc3)c3oc4ccccc4c23)cc1 ; c1ccc(-c2cc(-c3ccccc3)c3c(c2)oc2ccc(N(c4cccc5sc6ccccc6c45)c4ccc(-c5ccccc5)c5oc6ccccc6c45)cc23)cc1 ; c1ccc(-c2ccc(-c3ccccc3)c3c2oc2ccc(N(c4cccc5sc6ccccc6c45)c4ccc(-c5ccccc5)c5oc6ccccc6c45)cc23)cc1]

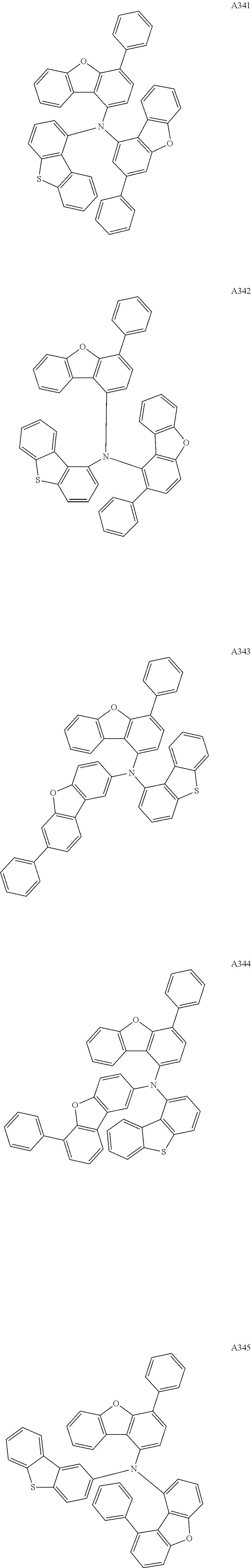 [SMILES: c1ccc(-c2cc(N(c3cccc4sc5ccccc5c34)c3ccc(-c4ccccc4)c4oc5ccccc5c34)c3c(c2)oc2ccccc23)cc1 ; c1ccc(-c2ccc3oc4ccccc4c3c2N(c2cccc3sc4ccccc4c23)c2ccc(-c3ccccc3)c3oc4ccccc4c23)cc1 ; c1ccc(-c2ccc3c(c2)oc2ccc(N(c4cccc5sc6ccccc6c45)c4ccc(-c5ccccc5)c5oc6ccccc6c45)cc23)cc1 ; c1ccc(-c2cccc3c2oc2ccc(N(c4cccc5sc6ccccc6c45)c4ccc(-c5ccccc5)c5oc6ccccc6c45)cc23)cc1 ; c1ccc(-c2ccc(N(c3ccc4sc5ccccc5c4c3)c3cccc4oc5cccc(-c6ccccc6)c5c34)c3c2oc2ccccc23)cc1]

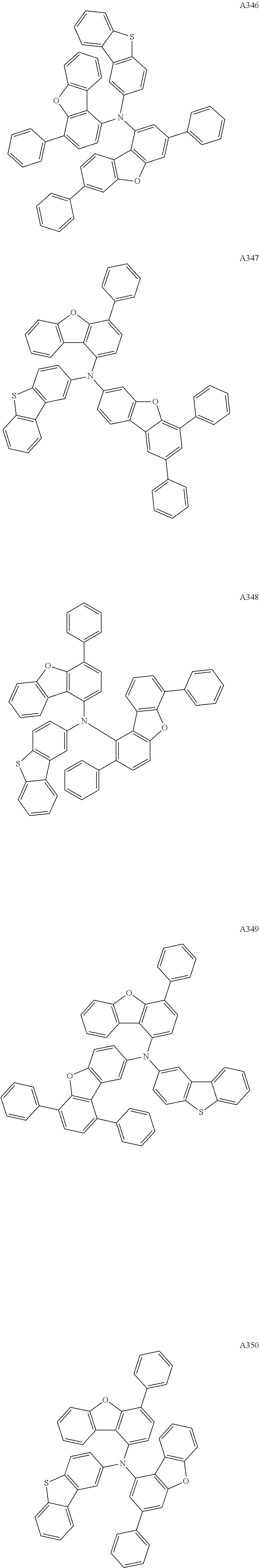 [SMILES: c1ccc(-c2ccc3c(c2)oc2cc(-c4ccccc4)cc(N(c4ccc5sc6ccccc6c5c4)c4ccc(-c5ccccc5)c5oc6ccccc6c45)c23)cc1 ; c1ccc(-c2cc(-c3ccccc3)c3oc4cc(N(c5ccc6sc7ccccc7c6c5)c5ccc(-c6ccccc6)c6oc7ccccc7c56)ccc4c3c2)cc1 ; c1ccc(-c2ccc3oc4c(-c5ccccc5)cccc4c3c2N(c2ccc3sc4ccccc4c3c2)c2ccc(-c3ccccc3)c3oc4ccccc4c23)cc1 ; c1ccc(-c2ccc(-c3ccccc3)c3c2oc2ccc(N(c4ccc5sc6ccccc6c5c4)c4ccc(-c5ccccc5)c5oc6ccccc6c45)cc23)cc1 ; c1ccc(-c2cc(N(c3ccc4sc5ccccc5c4c3)c3ccc(-c4ccccc4)c4oc5ccccc5c34)c3c(c2)oc2ccccc23)cc1]

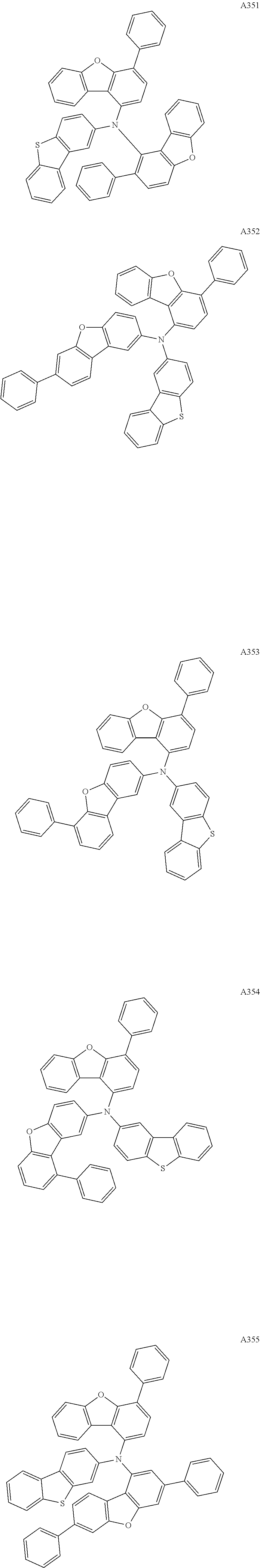 [SMILES: c1ccc(-c2ccc3oc4ccccc4c3c2N(c2ccc3sc4ccccc4c3c2)c2ccc(-c3ccccc3)c3oc4ccccc4c23)cc1 ; c1ccc(-c2ccc3c(c2)oc2ccc(N(c4ccc5sc6ccccc6c5c4)c4ccc(-c5ccccc5)c5oc6ccccc6c45)cc23)cc1 ; c1ccc(-c2cccc3c2oc2ccc(N(c4ccc5sc6ccccc6c5c4)c4ccc(-c5ccccc5)c5oc6ccccc6c45)cc23)cc1 ; c1ccc(-c2ccc(N(c3ccc4sc5ccccc5c4c3)c3ccc4oc5cccc(-c6ccccc6)c5c4c3)c3c2oc2ccccc23)cc1 ; c1ccc(-c2ccc3c(c2)oc2cc(-c4ccccc4)cc(N(c4ccc5c(c4)sc4ccccc45)c4ccc(-c5ccccc5)c5oc6ccccc6c45)c23)cc1]

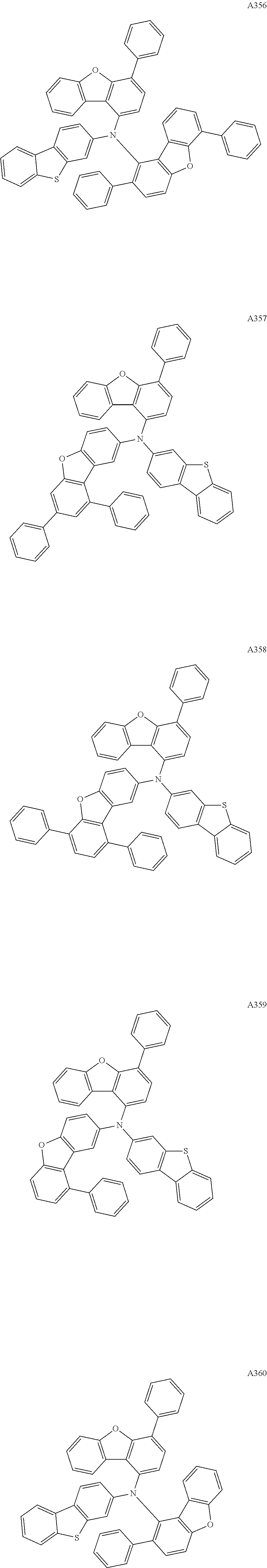 [SMILES: c1ccc(-c2ccc3oc4c(-c5ccccc5)cccc4c3c2N(c2ccc3c(c2)sc2ccccc23)c2ccc(-c3ccccc3)c3oc4ccccc4c23)cc1 ; c1ccc(-c2cc(-c3ccccc3)c3c(c2)oc2ccc(N(c4ccc5c(c4)sc4ccccc45)c4ccc(-c5ccccc5)c5oc6ccccc6c45)cc23)cc1 ; c1ccc(-c2ccc(-c3ccccc3)c3c2oc2ccc(N(c4ccc5c(c4)sc4ccccc45)c4ccc(-c5ccccc5)c5oc6ccccc6c45)cc23)cc1 ; c1ccc(-c2ccc(N(c3ccc4c(c3)sc3ccccc34)c3ccc4oc5cccc(-c6ccccc6)c5c4c3)c3c2oc2ccccc23)cc1 ; c1ccc(-c2ccc3oc4ccccc4c3c2N(c2ccc3c(c2)sc2ccccc23)c2ccc(-c3ccccc3)c3oc4ccccc4c23)cc1]

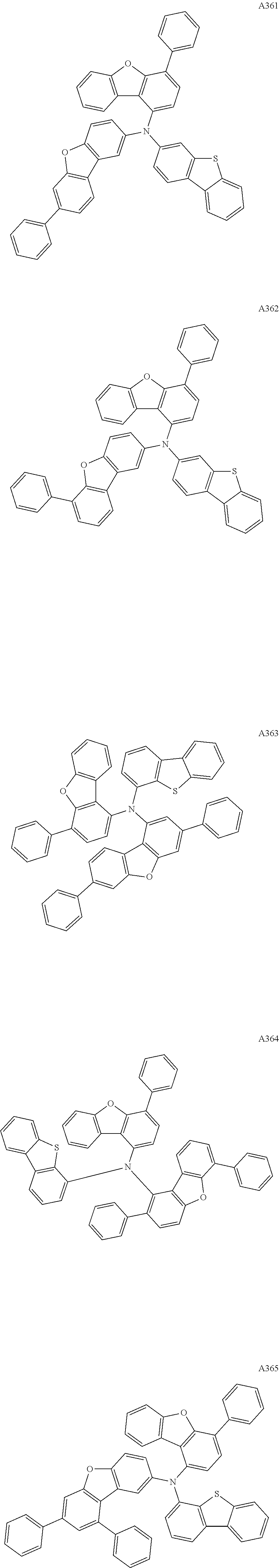 [SMILES: c1ccc(-c2ccc3c(c2)oc2ccc(N(c4ccc5c(c4)sc4ccccc45)c4ccc(-c5ccccc5)c5oc6ccccc6c45)cc23)cc1 ; c1ccc(-c2cccc3c2oc2ccc(N(c4ccc5c(c4)sc4ccccc45)c4ccc(-c5ccccc5)c5oc6ccccc6c45)cc23)cc1 ; c1ccc(-c2ccc3c(c2)oc2cc(-c4ccccc4)cc(N(c4cccc5c4sc4ccccc45)c4ccc(-c5ccccc5)c5oc6ccccc6c45)c23)cc1 ; c1ccc(-c2ccc3oc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)oc2ccc(N(c4cccc5c4sc4ccccc45)c4ccc(-c5ccccc5)c5oc6ccccc6c45)cc23)cc1]

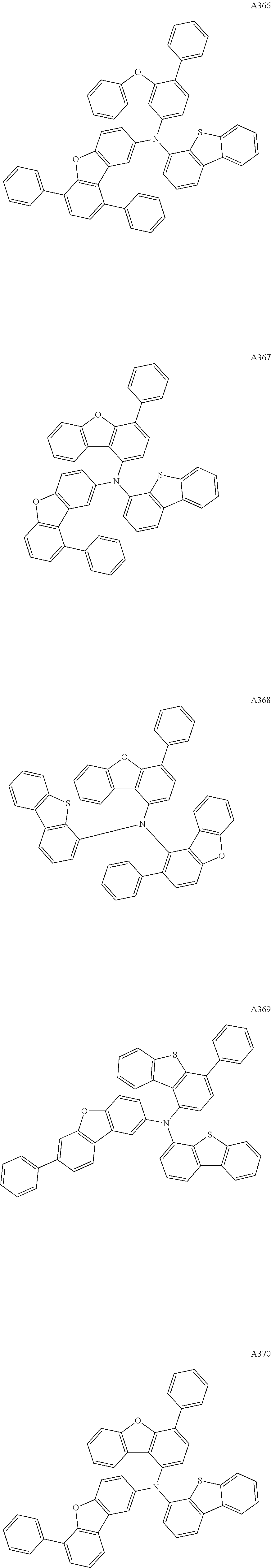 [SMILES: c1ccc(-c2ccc(-c3ccccc3)c3c2oc2ccc(N(c4cccc5c4sc4ccccc45)c4ccc(-c5ccccc5)c5oc6ccccc6c45)cc23)cc1 ; c1ccc(-c2ccc(N(c3ccc4oc5cccc(-c6ccccc6)c5c4c3)c3cccc4c3sc3ccccc34)c3c2oc2ccccc23)cc1 ; c1ccc(-c2ccc3oc4ccccc4c3c2N(c2cccc3c2sc2ccccc23)c2ccc(-c3ccccc3)c3oc4ccccc4c23)cc1 ; c1ccc(-c2ccc3c(c2)oc2ccc(N(c4cccc5c4sc4ccccc45)c4ccc(-c5ccccc5)c5sc6ccccc6c45)cc23)cc1 ; c1ccc(-c2cccc3c2oc2ccc(N(c4cccc5c4sc4ccccc45)c4ccc(-c5ccccc5)c5oc6ccccc6c45)cc23)cc1]

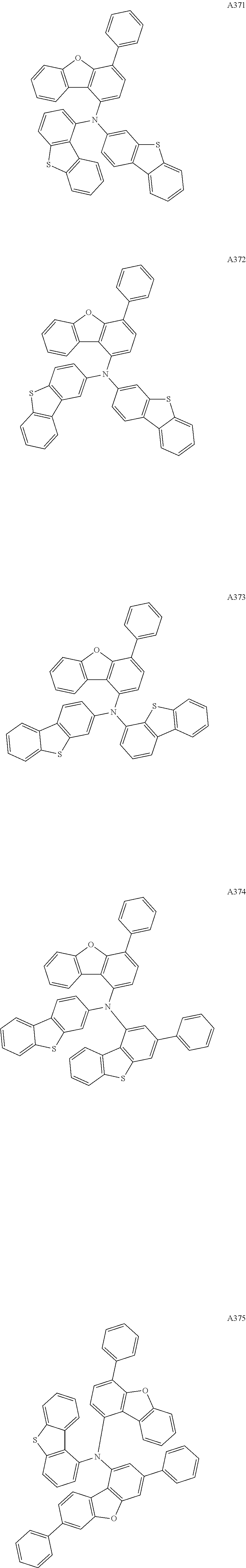 [SMILES: c1ccc(-c2ccc(N(c3ccc4c(c3)sc3ccccc34)c3cccc4sc5ccccc5c34)c3c2oc2ccccc23)cc1 ; c1ccc(-c2ccc(N(c3ccc4c(c3)sc3ccccc34)c3ccc4sc5ccccc5c4c3)c3c2oc2ccccc23)cc1 ; c1ccc(-c2ccc(N(c3ccc4c(c3)sc3ccccc34)c3cccc4c3sc3ccccc34)c3c2oc2ccccc23)cc1 ; c1ccc(-c2cc(N(c3ccc4c(c3)sc3ccccc34)c3ccc(-c4ccccc4)c4oc5ccccc5c34)c3c(c2)sc2ccccc23)cc1 ; c1ccc(-c2ccc3c(c2)oc2cc(-c4ccccc4)cc(N(c4cccc5sc6ccccc6c45)c4ccc(-c5ccccc5)c5oc6ccccc6c45)c23)cc1]

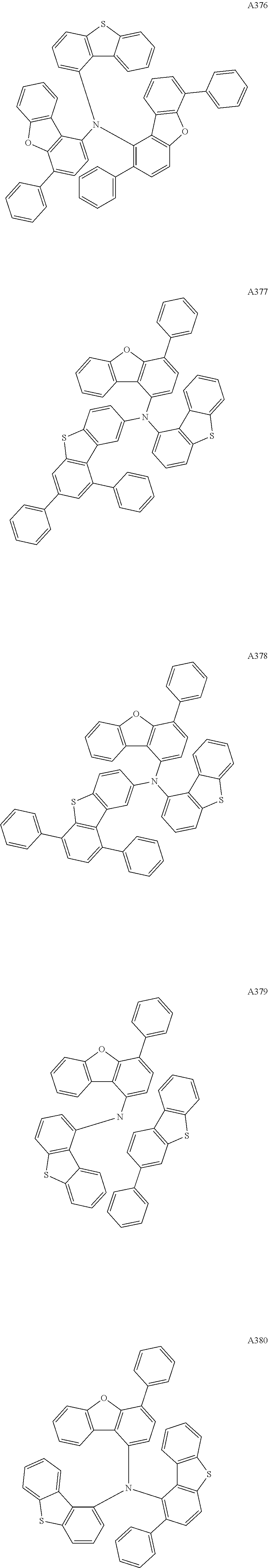

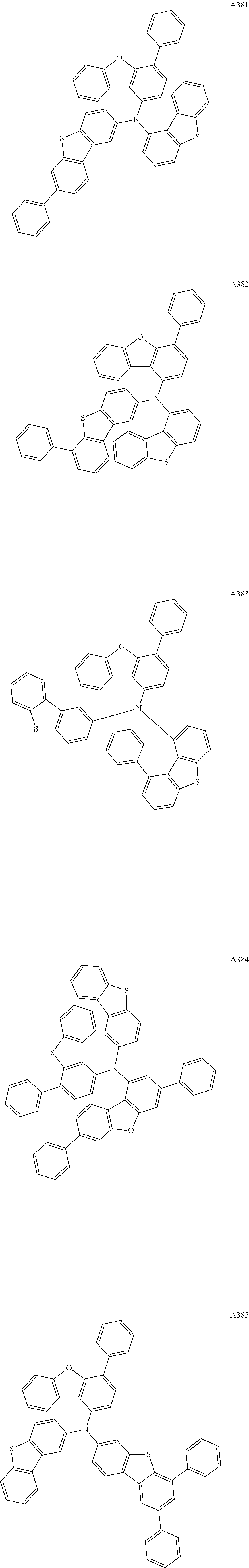 [SMILES: c1ccc(-c2ccc3c(c2)sc2ccc(N(c4cccc5sc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4sc5ccccc5c34)c3c2oc2ccccc23)cc1 ; c1ccc(-c2ccc(N(c3ccc4sc5ccccc5c4c3)c3cccc4sc5cccc(-c6ccccc6)c5c34)c3c2oc2ccccc23)cc1 ; c1ccc(-c2ccc3c(c2)oc2cc(-c4ccccc4)cc(N(c4ccc5sc6ccccc6c5c4)c4ccc(-c5ccccc5)c5sc6ccccc6c45)c23)cc1 ; c1ccc(-c2cc(-c3ccccc3)c3sc4cc(N(c5ccc6sc7ccccc7c6c5)c5ccc(-c6ccccc6)c6oc7ccccc7c56)ccc4c3c2)cc1]

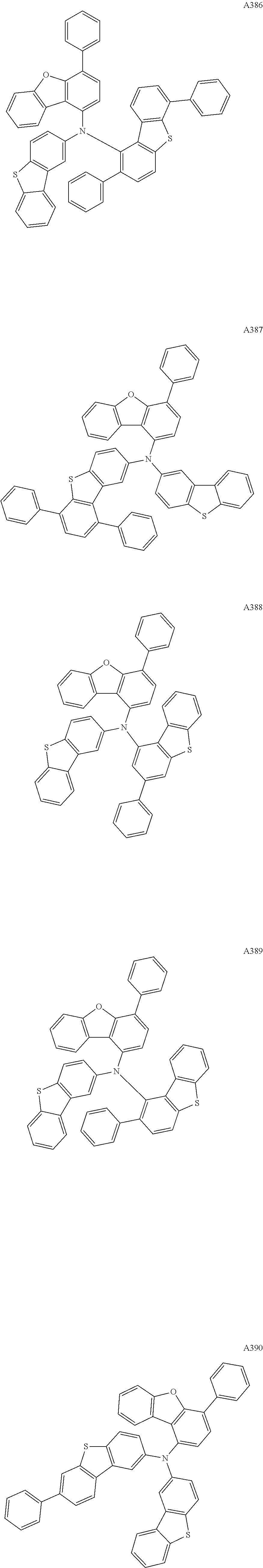 [SMILES: c1ccc(-c2ccc3sc4c(-c5ccccc5)cccc4c3c2N(c2ccc3sc4ccccc4c3c2)c2ccc(-c3ccccc3)c3oc4ccccc4c23)cc1 ; c1ccc(-c2ccc(N(c3ccc4sc5ccccc5c4c3)c3ccc4sc5c(-c6ccccc6)ccc(-c6ccccc6)c5c4c3)c3c2oc2ccccc23)cc1 ; c1ccc(-c2cc(N(c3ccc4sc5ccccc5c4c3)c3ccc(-c4ccccc4)c4oc5ccccc5c34)c3c(c2)sc2ccccc23)cc1 ; c1ccc(-c2ccc3sc4ccccc4c3c2N(c2ccc3sc4ccccc4c3c2)c2ccc(-c3ccccc3)c3oc4ccccc4c23)cc1 ; c1ccc(-c2ccc3c(c2)sc2ccc(N(c4ccc5sc6ccccc6c5c4)c4ccc(-c5ccccc5)c5oc6ccccc6c45)cc23)cc1]

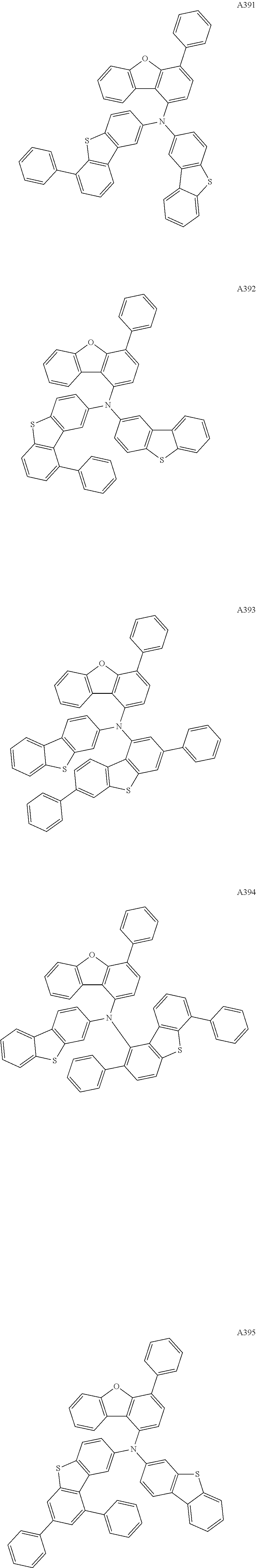 [SMILES: c1ccc(-c2ccc(N(c3ccc4sc5ccccc5c4c3)c3ccc4sc5c(-c6ccccc6)cccc5c4c3)c3c2oc2ccccc23)cc1 ; c1ccc(-c2ccc(N(c3ccc4sc5ccccc5c4c3)c3ccc4sc5cccc(-c6ccccc6)c5c4c3)c3c2oc2ccccc23)cc1 ; c1ccc(-c2ccc3c(c2)sc2cc(-c4ccccc4)cc(N(c4ccc5c(c4)sc4ccccc45)c4ccc(-c5ccccc5)c5oc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3oc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5oc6ccccc6c45)cc23)cc1]

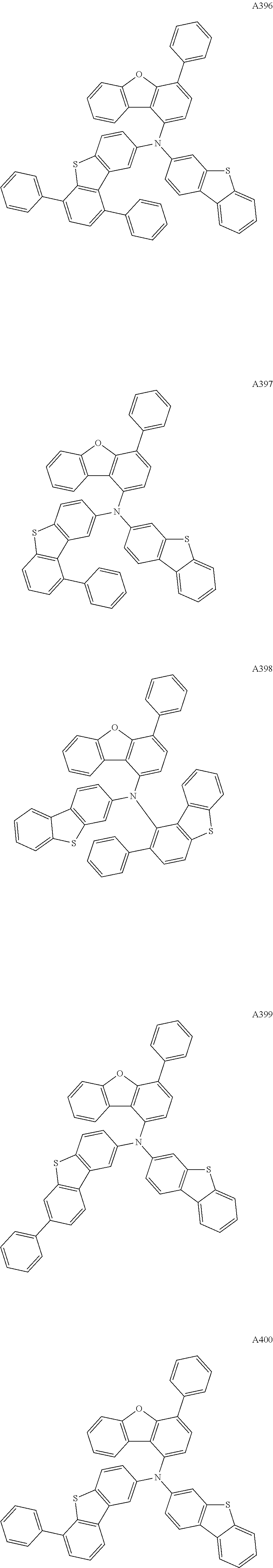 [SMILES: c1ccc(-c2ccc(N(c3ccc4c(c3)sc3ccccc34)c3ccc4sc5c(-c6ccccc6)ccc(-c6ccccc6)c5c4c3)c3c2oc2ccccc23)cc1 ; c1ccc(-c2ccc(N(c3ccc4c(c3)sc3ccccc34)c3ccc4sc5cccc(-c6ccccc6)c5c4c3)c3c2oc2ccccc23)cc1 ; c1ccc(-c2ccc3sc4ccccc4c3c2N(c2ccc3c(c2)sc2ccccc23)c2ccc(-c3ccccc3)c3oc4ccccc4c23)cc1 ; c1ccc(-c2ccc3c(c2)sc2ccc(N(c4ccc5c(c4)sc4ccccc45)c4ccc(-c5ccccc5)c5oc6ccccc6c45)cc23)cc1 ; c1ccc(-c2ccc(N(c3ccc4c(c3)sc3ccccc34)c3ccc4sc5c(-c6ccccc6)cccc5c4c3)c3c2oc2ccccc23)cc1]

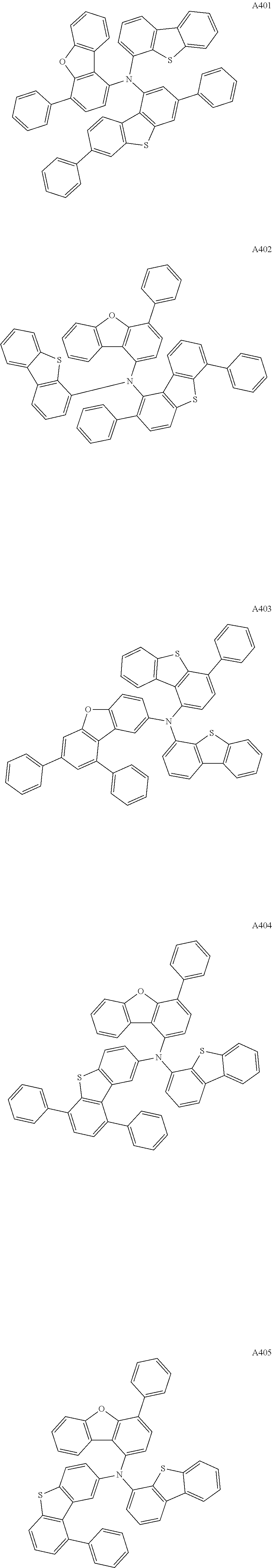 [SMILES: c1ccc(-c2ccc3c(c2)sc2cc(-c4ccccc4)cc(N(c4cccc5c4sc4ccccc45)c4ccc(-c5ccccc5)c5oc6ccccc6c45)c23)cc1 ; c1ccc(-c2ccc3sc4c(-c5ccccc5)cccc4c3c2N(c2cccc3c2sc2ccccc23)c2ccc(-c3ccccc3)c3oc4ccccc4c23)cc1 ; c1ccc(-c2cc(-c3ccccc3)c3c(c2)oc2ccc(N(c4cccc5c4sc4ccccc45)c4ccc(-c5ccccc5)c5sc6ccccc6c45)cc23)cc1 ; c1ccc(-c2ccc(N(c3ccc4sc5c(-c6ccccc6)ccc(-c6ccccc6)c5c4c3)c3cccc4c3sc3ccccc34)c3c2oc2ccccc23)cc1 ; c1ccc(-c2ccc(N(c3ccc4sc5cccc(-c6ccccc6)c5c4c3)c3cccc4c3sc3ccccc34)c3c2oc2ccccc23)cc1]

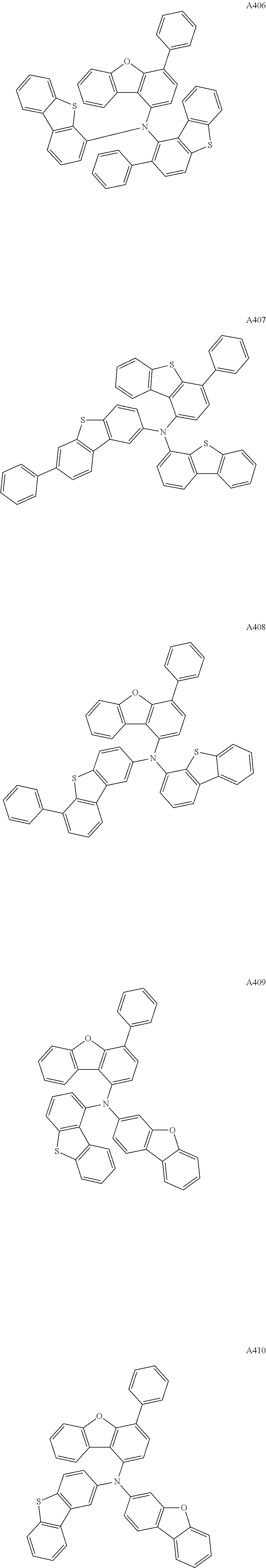 [SMILES: c1ccc(-c2ccc3sc4ccccc4c3c2N(c2cccc3c2sc2ccccc23)c2ccc(-c3ccccc3)c3oc4ccccc4c23)cc1 ; c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2ccc(N(c3ccc4sc5c(-c6ccccc6)cccc5c4c3)c3cccc4c3sc3ccccc34)c3c2oc2ccccc23)cc1 ; c1ccc(-c2ccc(N(c3ccc4c(c3)oc3ccccc34)c3cccc4sc5ccccc5c34)c3c2oc2ccccc23)cc1 ; c1ccc(-c2ccc(N(c3ccc4c(c3)oc3ccccc34)c3ccc4sc5ccccc5c4c3)c3c2oc2ccccc23)cc1]

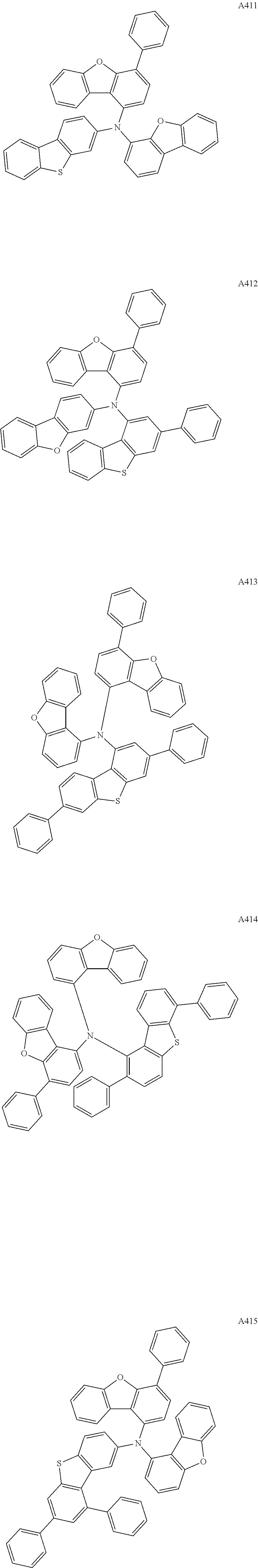 [SMILES: c1ccc(-c2ccc(N(c3ccc4c(c3)sc3ccccc34)c3cccc4c3oc3ccccc34)c3c2oc2ccccc23)cc1 ; c1ccc(-c2cc(N(c3ccc4c(c3)oc3ccccc34)c3ccc(-c4ccccc4)c4oc5ccccc5c34)c3c(c2)sc2ccccc23)cc1 ; c1ccc(-c2ccc3c(c2)sc2cc(-c4ccccc4)cc(N(c4cccc5oc6ccccc6c45)c4ccc(-c5ccccc5)c5oc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3oc4ccccc4c23)cc1 ; c1ccc(-c2cc(-c3ccccc3)c3c(c2)sc2ccc(N(c4cccc5oc6ccccc6c45)c4ccc(-c5ccccc5)c5oc6ccccc6c45)cc23)cc1]

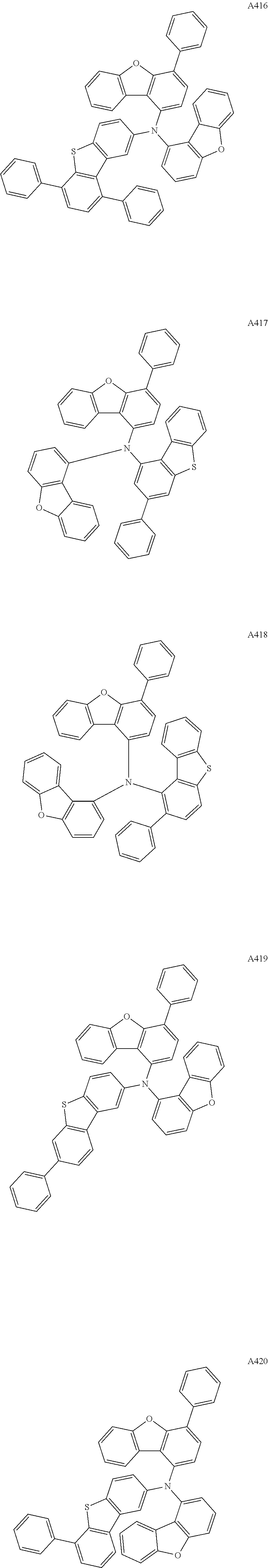 [SMILES: c1ccc(-c2ccc(N(c3ccc4sc5c(-c6ccccc6)ccc(-c6ccccc6)c5c4c3)c3cccc4oc5ccccc5c34)c3c2oc2ccccc23)cc1 ; c1ccc(-c2cc(N(c3cccc4oc5ccccc5c34)c3ccc(-c4ccccc4)c4oc5ccccc5c34)c3c(c2)sc2ccccc23)cc1 ; c1ccc(-c2ccc3sc4ccccc4c3c2N(c2cccc3oc4ccccc4c23)c2ccc(-c3ccccc3)c3oc4ccccc4c23)cc1 ; c1ccc(-c2ccc3c(c2)sc2ccc(N(c4cccc5oc6ccccc6c45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4oc5ccccc5c34)c3c2oc2ccccc23)cc1]

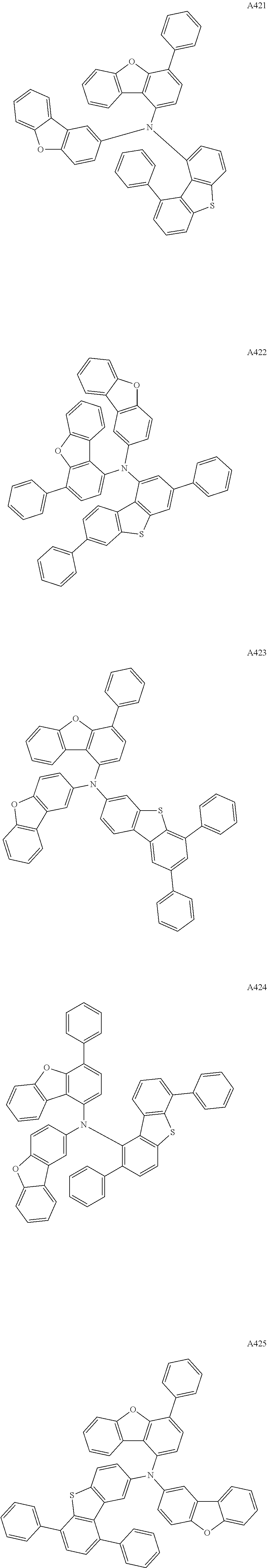 [SMILES: c1ccc(-c2ccc(N(c3ccc4oc5ccccc5c4c3)c3cccc4sc5cccc(-c6ccccc6)c5c34)c3c2oc2ccccc23)cc1 ; c1ccc(-c2ccc3c(c2)sc2cc(-c4ccccc4)cc(N(c4ccc5oc6ccccc6c5c4)c4ccc(-c5ccccc5)c5oc6ccccc6c45)c23)cc1 ; c1ccc(-c2cc(-c3ccccc3)c3sc4cc(N(c5ccc6oc7ccccc7c6c5)c5ccc(-c6ccccc6)c6oc7ccccc7c56)ccc4c3c2)cc1 ; c1ccc(-c2ccc3sc4c(-c5ccccc5)cccc4c3c2N(c2ccc3oc4ccccc4c3c2)c2ccc(-c3ccccc3)c3oc4ccccc4c23)cc1 ; c1ccc(-c2ccc(N(c3ccc4oc5ccccc5c4c3)c3ccc4sc5c(-c6ccccc6)ccc(-c6ccccc6)c5c4c3)c3c2oc2ccccc23)cc1]

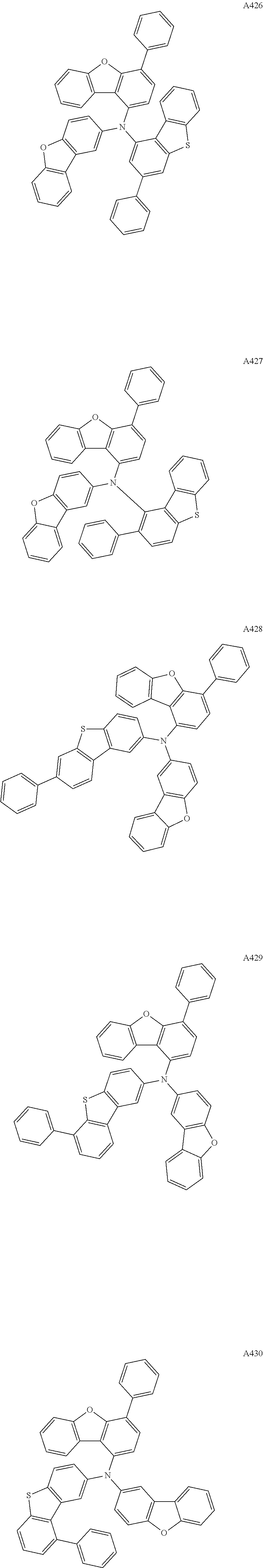 [SMILES: c1ccc(-c2cc(N(c3ccc4oc5ccccc5c4c3)c3ccc(-c4ccccc4)c4oc5ccccc5c34)c3c(c2)sc2ccccc23)cc1 ; c1ccc(-c2ccc3sc4ccccc4c3c2N(c2ccc3oc4ccccc4c3c2)c2ccc(-c3ccccc3)c3oc4ccccc4c23)cc1 ; c1ccc(-c2ccc3c(c2)sc2ccc(N(c4ccc5oc6ccccc6c5c4)c4ccc(-c5ccccc5)c5oc6ccccc6c45)cc23)cc1 ; c1ccc(-c2ccc(N(c3ccc4oc5ccccc5c4c3)c3ccc4sc5c(-c6ccccc6)cccc5c4c3)c3c2oc2ccccc23)cc1 ; c1ccc(-c2ccc(N(c3ccc4oc5ccccc5c4c3)c3ccc4sc5cccc(-c6ccccc6)c5c4c3)c3c2oc2ccccc23)cc1]

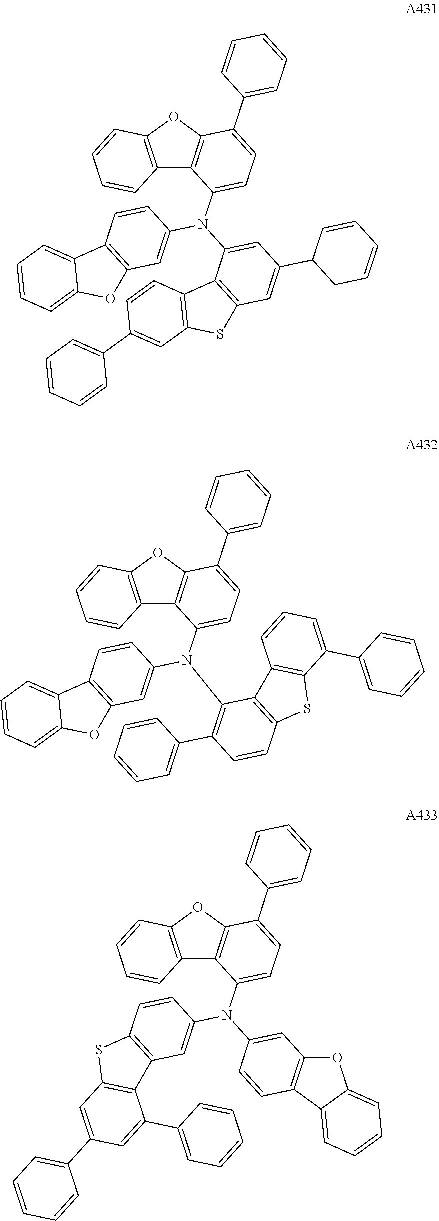 [SMILES: C1=CCC(c2cc(N(c3ccc4c(c3)oc3ccccc34)c3ccc(-c4ccccc4)c4oc5ccccc5c34)c3c(c2)sc2cc(-c4ccccc4)ccc23)C=C1 ; c1ccc(-c2ccc3sc4c(-c5ccccc5)cccc4c3c2N(c2ccc3c(c2)oc2ccccc23)c2ccc(-c3ccccc3)c3oc4ccccc4c23)cc1 ; c1ccc(-c2cc(-c3ccccc3)c3c(c2)sc2ccc(N(c4ccc5c(c4)oc4ccccc45)c4ccc(-c5ccccc5)c5oc6ccccc6c45)cc23)cc1]

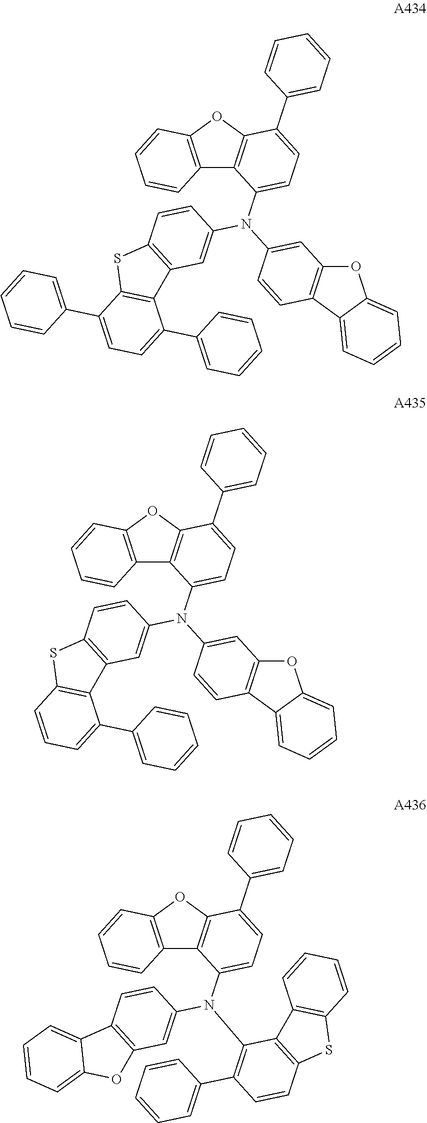 [SMILES: c1ccc(-c2ccc(N(c3ccc4c(c3)oc3ccccc34)c3ccc4sc5c(-c6ccccc6)ccc(-c6ccccc6)c5c4c3)c3c2oc2ccccc23)cc1 ; c1ccc(-c2ccc(N(c3ccc4c(c3)oc3ccccc34)c3ccc4sc5cccc(-c6ccccc6)c5c4c3)c3c2oc2ccccc23)cc1 ; c1ccc(-c2ccc3sc4ccccc4c3c2N(c2ccc3c(c2)oc2ccccc23)c2ccc(-c3ccccc3)c3oc4ccccc4c23)cc1]

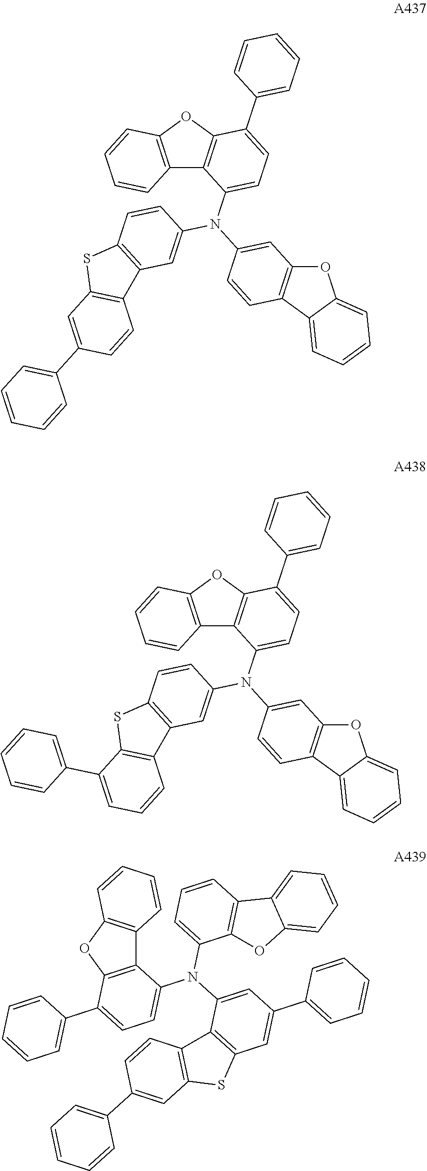 [SMILES: c1ccc(-c2ccc3c(c2)sc2ccc(N(c4ccc5c(c4)oc4ccccc45)c4ccc(-c5ccccc5)c5oc6ccccc6c45)cc23)cc1 ; c1ccc(-c2ccc(N(c3ccc4c(c3)oc3ccccc34)c3ccc4sc5c(-c6ccccc6)cccc5c4c3)c3c2oc2ccccc23)cc1 ; c1ccc(-c2ccc3c(c2)sc2cc(-c4ccccc4)cc(N(c4cccc5c4oc4ccccc45)c4ccc(-c5ccccc5)c5oc6ccccc6c45)c23)cc1]

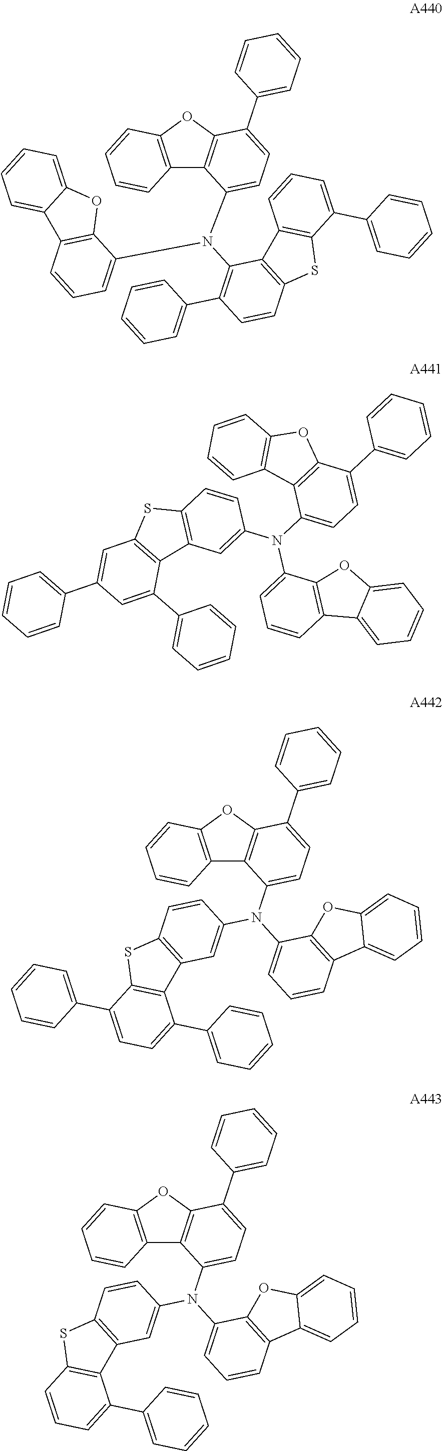

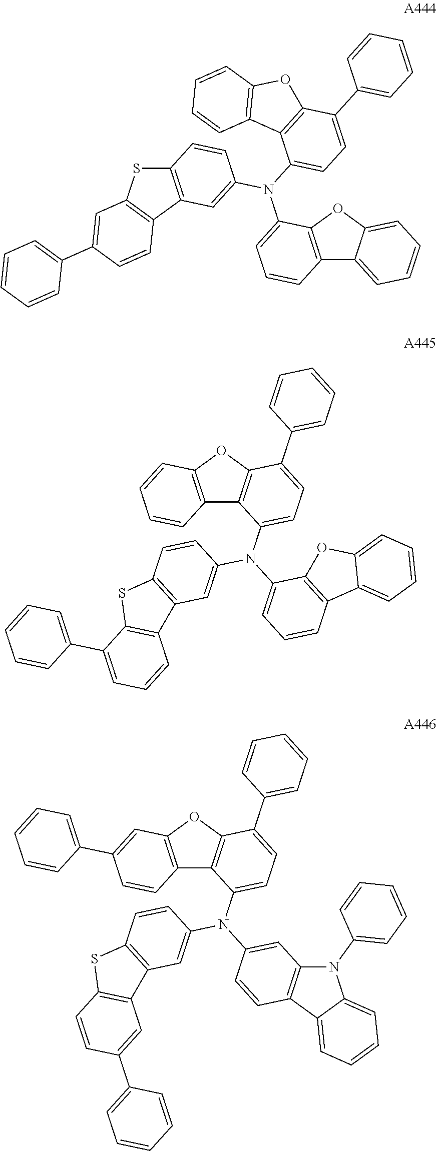 [SMILES: c1ccc(-c2ccc3c(c2)sc2ccc(N(c4cccc5c4oc4ccccc45)c4ccc(-c5ccccc5)c5oc6ccccc6c45)cc23)cc1 ; c1ccc(-c2ccc(N(c3ccc4sc5c(-c6ccccc6)cccc5c4c3)c3cccc4c3oc3ccccc34)c3c2oc2ccccc23)cc1 ; c1ccc(-c2ccc3c(c2)oc2c(-c4ccccc4)ccc(N(c4ccc5sc6ccc(-c7ccccc7)cc6c5c4)c4ccc5c6ccccc6n(-c6ccccc6)c5c4)c23)cc1]

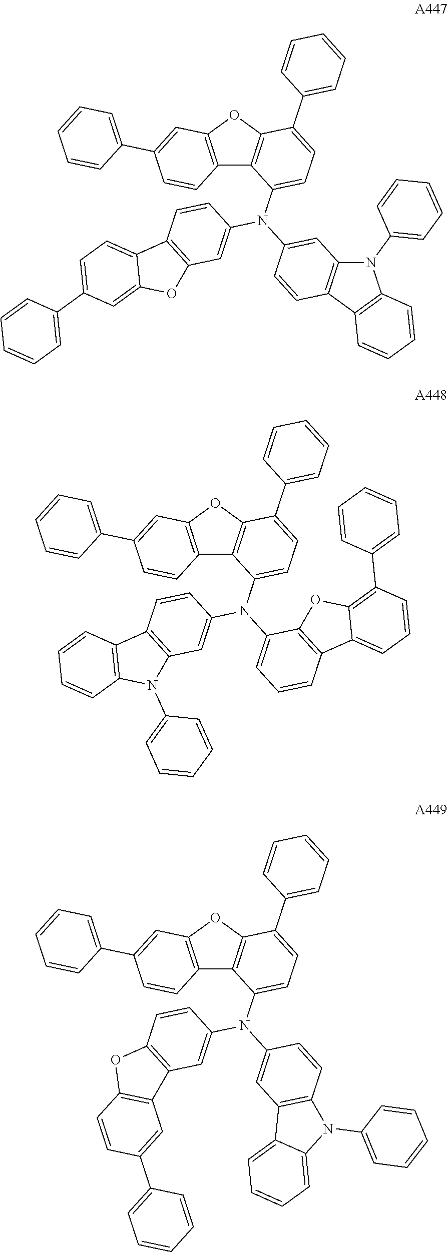 [SMILES: c1ccc(-c2ccc3c(c2)oc2cc(N(c4ccc5c6ccccc6n(-c6ccccc6)c5c4)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4ccc5c6ccccc6n(-c6ccccc6)c5c4)c4cccc5c4oc4c(-c6ccccc6)cccc45)c23)cc1 ; c1ccc(-c2ccc3c(c2)oc2c(-c4ccccc4)ccc(N(c4ccc5oc6ccc(-c7ccccc7)cc6c5c4)c4ccc5c(c4)c4ccccc4n5-c4ccccc4)c23)cc1]

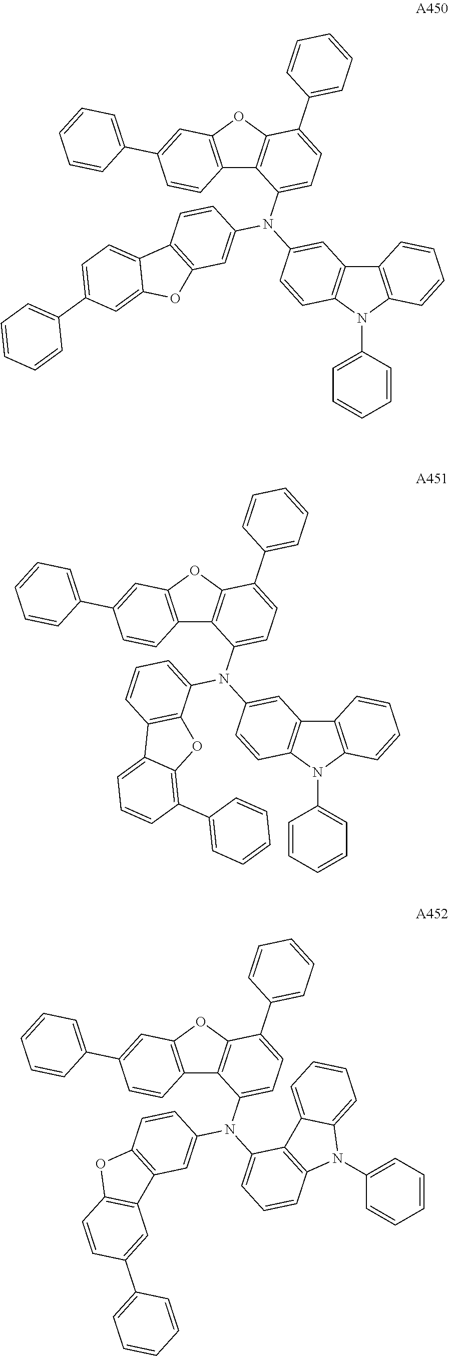 [SMILES: c1ccc(-c2ccc3c(c2)oc2cc(N(c4ccc5c(c4)c4ccccc4n5-c4ccccc4)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4ccc5c(c4)c4ccccc4n5-c4ccccc4)c4cccc5c4oc4c(-c6ccccc6)cccc45)c23)cc1 ; c1ccc(-c2ccc3c(c2)oc2c(-c4ccccc4)ccc(N(c4ccc5oc6ccc(-c7ccccc7)cc6c5c4)c4cccc5c4c4ccccc4n5-c4ccccc4)c23)cc1]

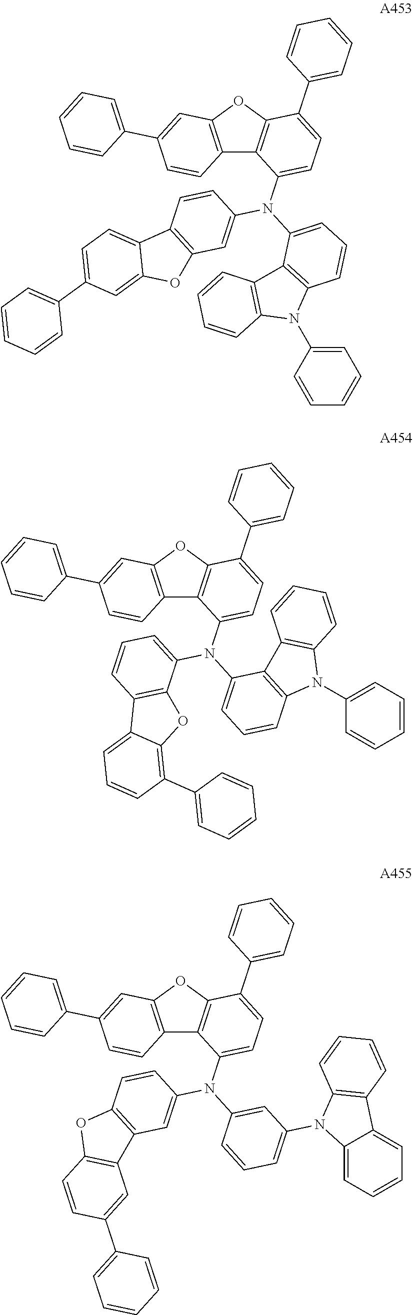 [SMILES: c1ccc(-c2ccc3c(c2)oc2cc(N(c4ccc(-c5ccccc5)c5oc6cc(-c7ccccc7)ccc6c45)c4cccc5c4c4ccccc4n5-c4ccccc4)ccc23)cc1 ; c1ccc(-c2ccc3c(c2)oc2c(-c4ccccc4)ccc(N(c4cccc5c4oc4c(-c6ccccc6)cccc45)c4cccc5c4c4ccccc4n5-c4ccccc4)c23)cc1 ; c1ccc(-c2ccc3c(c2)oc2c(-c4ccccc4)ccc(N(c4cccc(-n5c6ccccc6c6ccccc65)c4)c4ccc5oc6ccc(-c7ccccc7)cc6c5c4)c23)cc1]

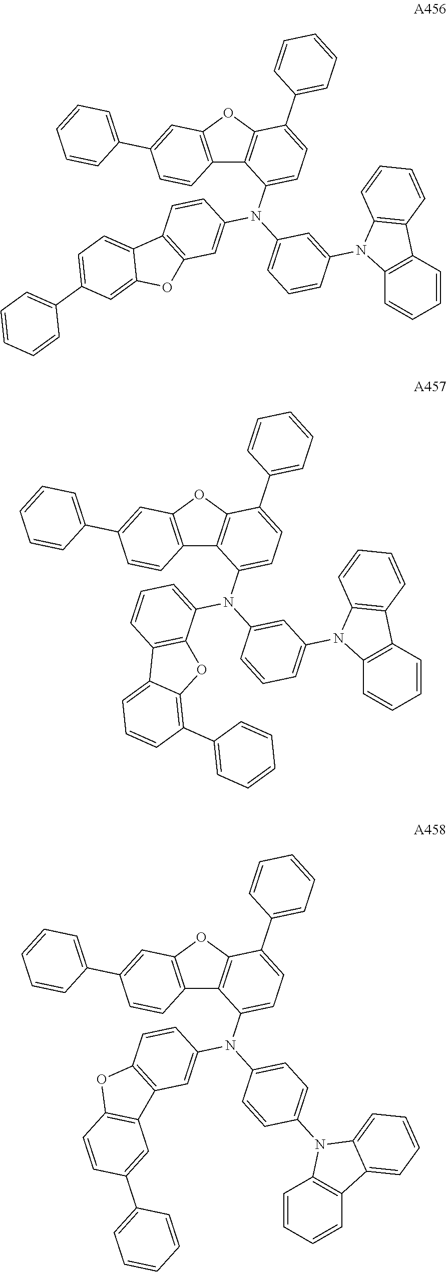 [SMILES: c1ccc(-c2ccc3c(c2)oc2cc(N(c4cccc(-n5c6ccccc6c6ccccc65)c4)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4cccc(-n5c6ccccc6c6ccccc65)c4)c4cccc5c4oc4c(-c6ccccc6)cccc45)c23)cc1 ; c1ccc(-c2ccc3c(c2)oc2c(-c4ccccc4)ccc(N(c4ccc(-n5c6ccccc6c6ccccc65)cc4)c4ccc5oc6ccc(-c7ccccc7)cc6c5c4)c23)cc1]

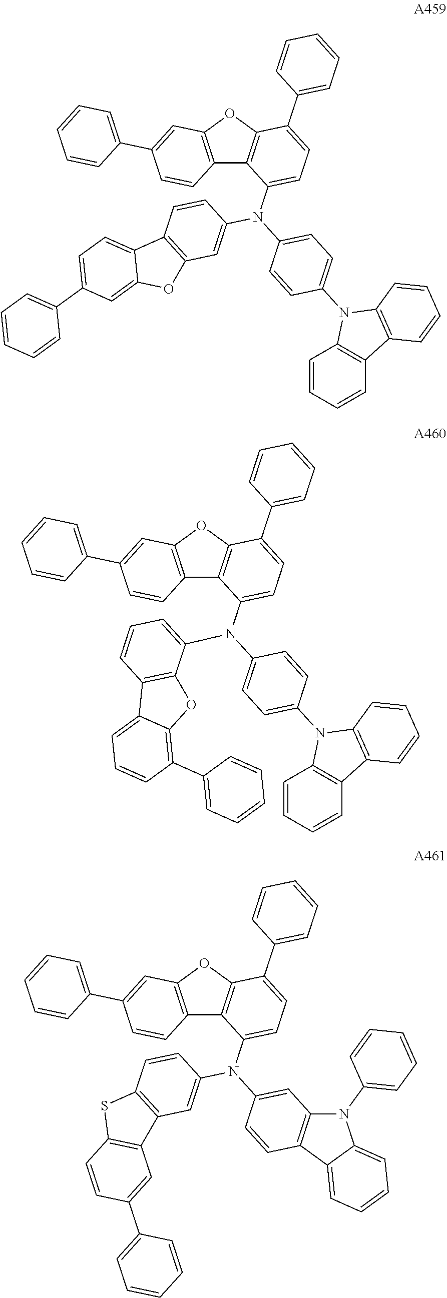

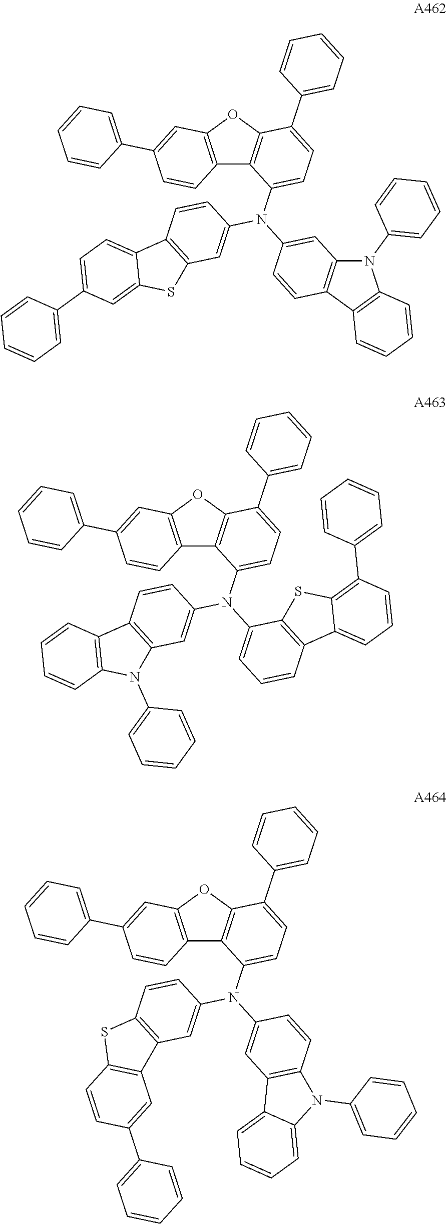

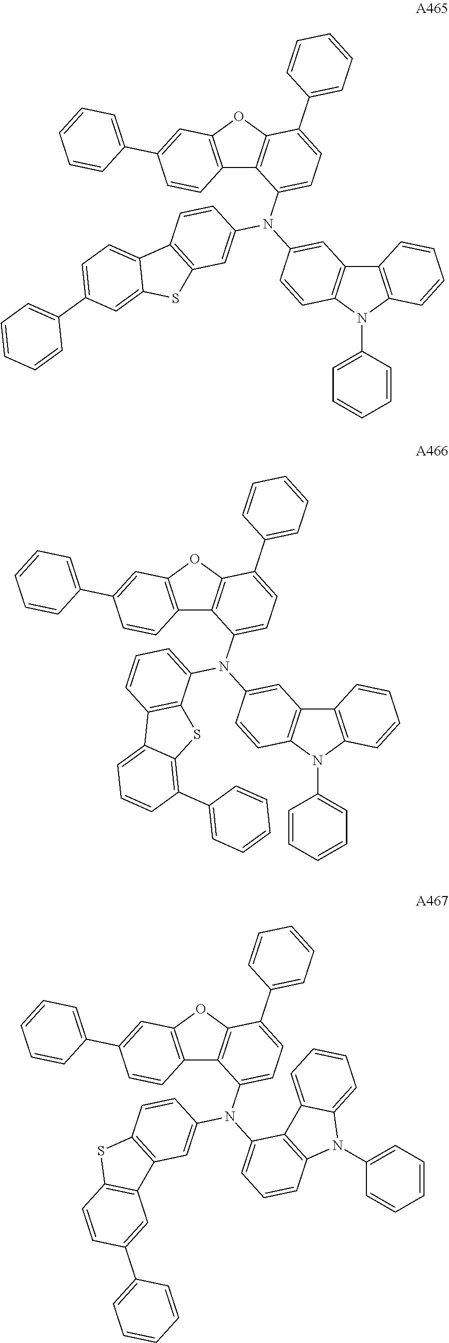 [SMILES: c1ccc(-c2ccc3c(c2)oc2c(-c4ccccc4)ccc(N(c4ccc5c(c4)sc4cc(-c6ccccc6)ccc45)c4ccc5c(c4)c4ccccc4n5-c4ccccc4)c23)cc1 ; c1ccc(-c2ccc3c(c2)oc2c(-c4ccccc4)ccc(N(c4ccc5c(c4)c4ccccc4n5-c4ccccc4)c4cccc5c4sc4c(-c6ccccc6)cccc45)c23)cc1 ; c1ccc(-c2ccc3c(c2)oc2c(-c4ccccc4)ccc(N(c4ccc5sc6ccc(-c7ccccc7)cc6c5c4)c4cccc5c4c4ccccc4n5-c4ccccc4)c23)cc1]

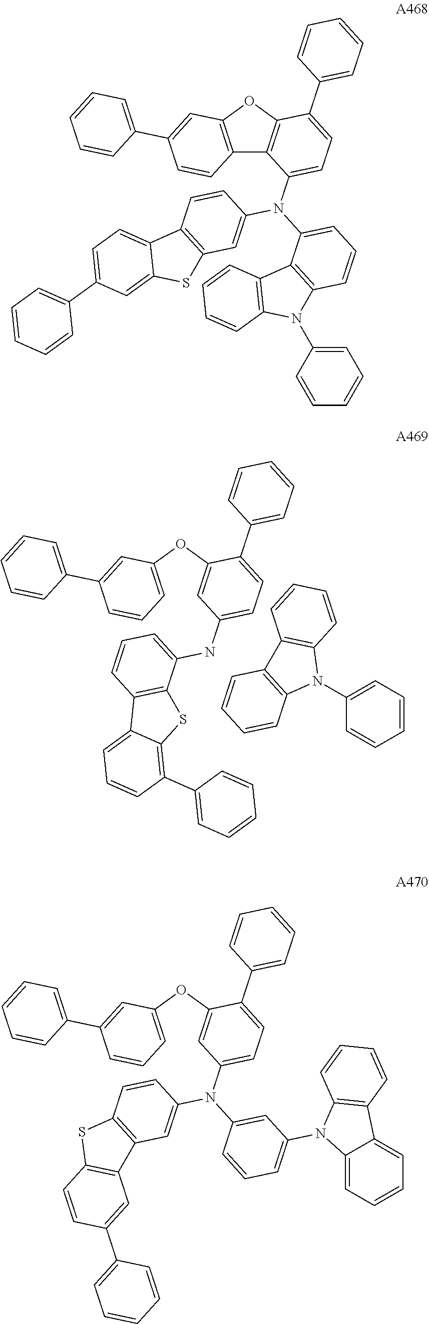

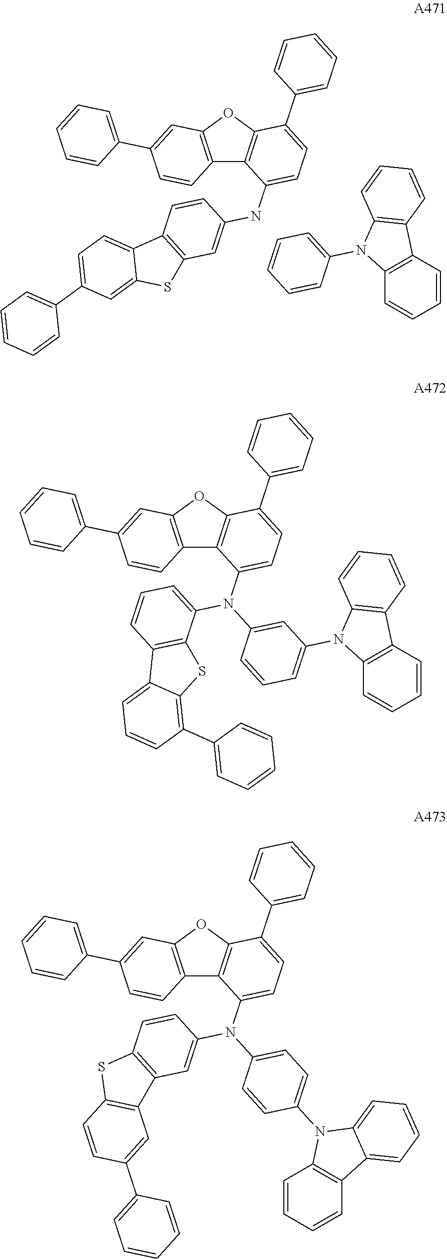

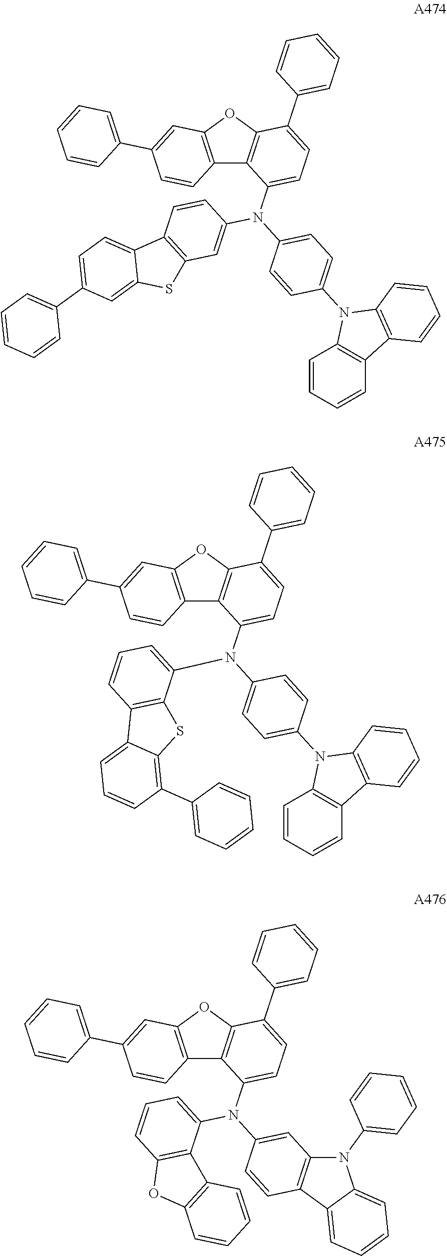 [SMILES: c1ccc(-c2ccc3c(c2)oc2c(-c4ccccc4)ccc(N(c4ccc(-n5c6ccccc6c6ccccc65)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-n5c6ccccc6c6ccccc65)cc4)c4cccc5c4sc4c(-c6ccccc6)cccc45)c23)cc1 ; c1ccc(-c2ccc3c(c2)oc2c(-c4ccccc4)ccc(N(c4ccc5c6ccccc6n(-c6ccccc6)c5c4)c4cccc5oc6ccccc6c45)c23)cc1]

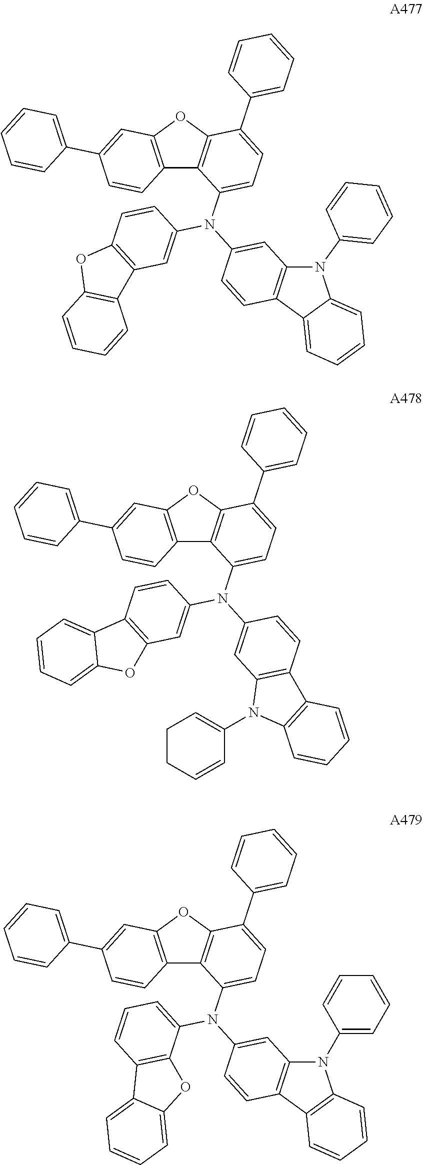 [SMILES: c1ccc(-c2ccc3c(c2)oc2c(-c4ccccc4)ccc(N(c4ccc5oc6ccccc6c5c4)c4ccc5c6ccccc6n(-c6ccccc6)c5c4)c23)cc1 ; C1=CC(n2c3ccccc3c3ccc(N(c4ccc5c(c4)oc4ccccc45)c4ccc(-c5ccccc5)c5oc6cc(-c7ccccc7)ccc6c45)cc32)=CCC1 ; c1ccc(-c2ccc3c(c2)oc2c(-c4ccccc4)ccc(N(c4ccc5c6ccccc6n(-c6ccccc6)c5c4)c4cccc5c4oc4ccccc45)c23)cc1]

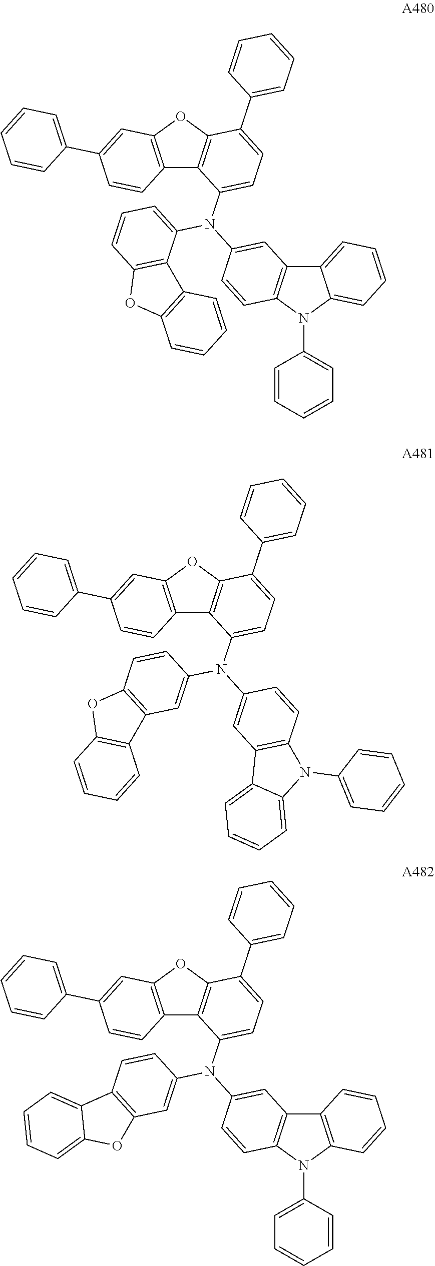

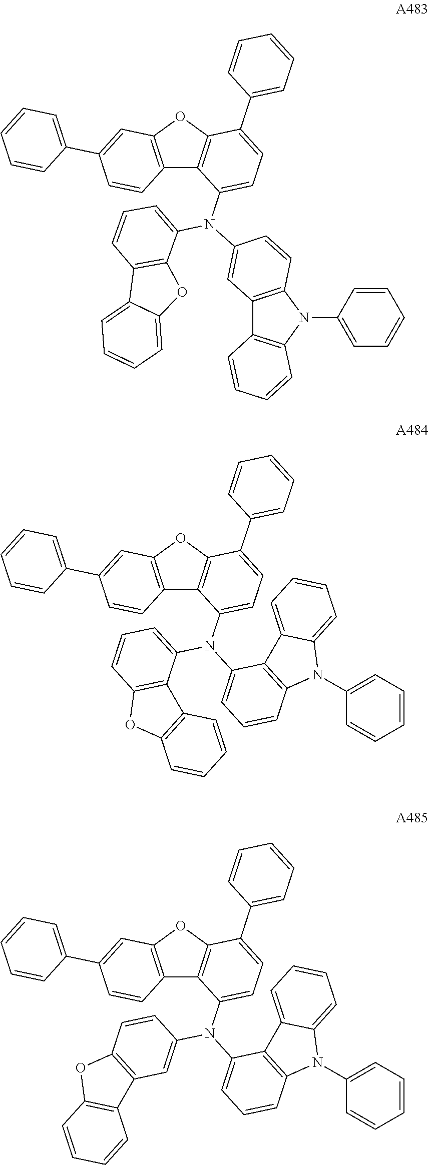 [SMILES: c1ccc(-c2ccc3c(c2)oc2c(-c4ccccc4)ccc(N(c4ccc5c(c4)c4ccccc4n5-c4ccccc4)c4cccc5c4oc4ccccc45)c23)cc1 ; c1ccc(-c2ccc3c(c2)oc2c(-c4ccccc4)ccc(N(c4cccc5oc6ccccc6c45)c4cccc5c4c4ccccc4n5-c4ccccc4)c23)cc1 ; c1ccc(-c2ccc3c(c2)oc2c(-c4ccccc4)ccc(N(c4ccc5oc6ccccc6c5c4)c4cccc5c4c4ccccc4n5-c4ccccc4)c23)cc1]

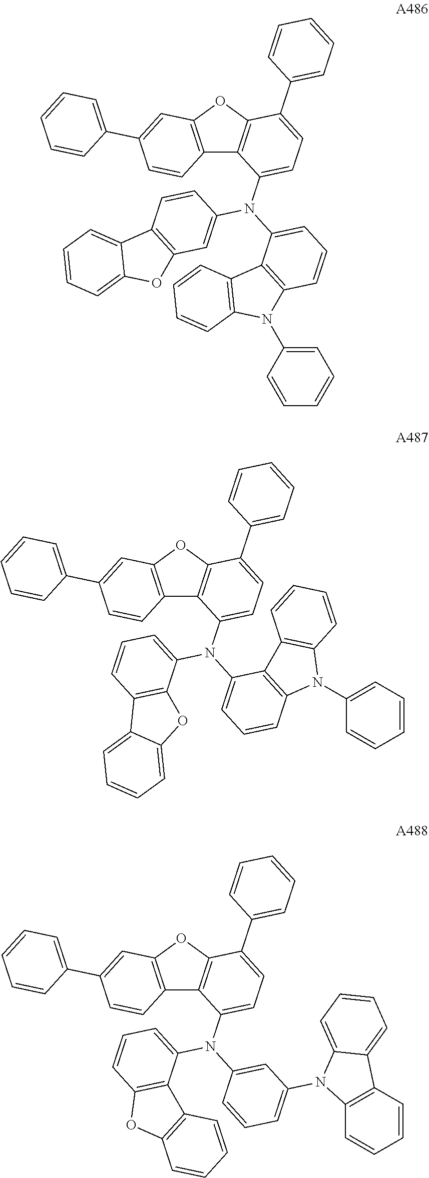 [SMILES: c1ccc(-c2ccc3c(c2)oc2c(-c4ccccc4)ccc(N(c4ccc5c(c4)oc4ccccc45)c4cccc5c4c4ccccc4n5-c4ccccc4)c23)cc1 ; c1ccc(-c2ccc3c(c2)oc2c(-c4ccccc4)ccc(N(c4cccc5c4oc4ccccc45)c4cccc5c4c4ccccc4n5-c4ccccc4)c23)cc1 ; c1ccc(-c2ccc3c(c2)oc2c(-c4ccccc4)ccc(N(c4cccc(-n5c6ccccc6c6ccccc65)c4)c4cccc5oc6ccccc6c45)c23)cc1]

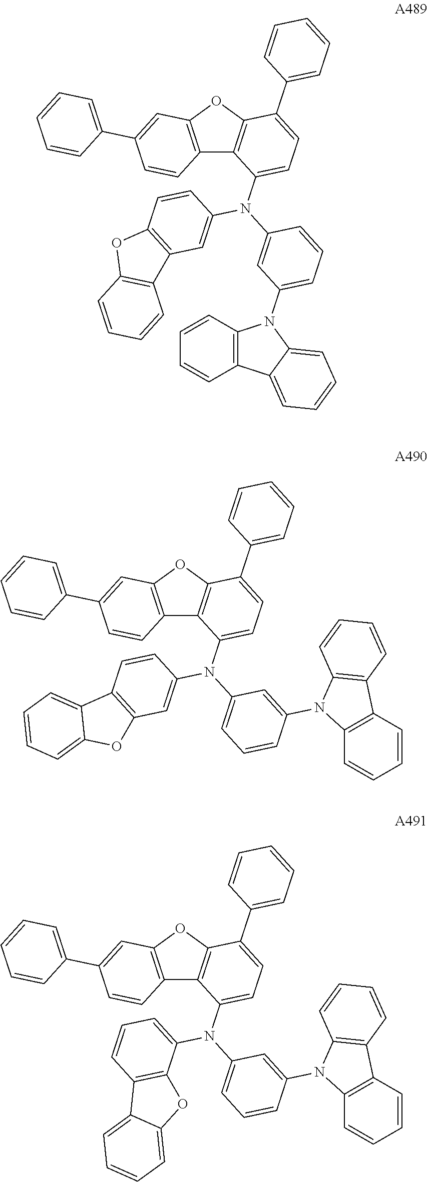 [SMILES: c1ccc(-c2ccc3c(c2)oc2c(-c4ccccc4)ccc(N(c4cccc(-n5c6ccccc6c6ccccc65)c4)c4ccc5oc6ccccc6c5c4)c23)cc1 ; c1ccc(-c2ccc3c(c2)oc2c(-c4ccccc4)ccc(N(c4cccc(-n5c6ccccc6c6ccccc65)c4)c4ccc5c(c4)oc4ccccc45)c23)cc1 ; c1ccc(-c2ccc3c(c2)oc2c(-c4ccccc4)ccc(N(c4cccc(-n5c6ccccc6c6ccccc65)c4)c4cccc5c4oc4ccccc45)c23)cc1]

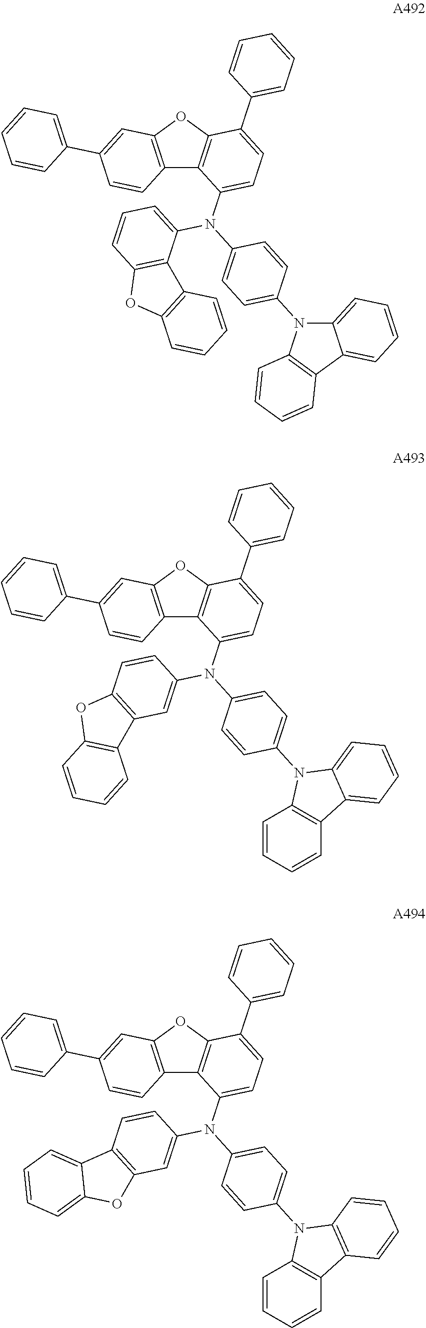 [SMILES: c1ccc(-c2ccc3c(c2)oc2c(-c4ccccc4)ccc(N(c4ccc(-n5c6ccccc6c6ccccc65)cc4)c4cccc5oc6ccccc6c45)c23)cc1 ; c1ccc(-c2ccc3c(c2)oc2c(-c4ccccc4)ccc(N(c4ccc(-n5c6ccccc6c6ccccc65)cc4)c4ccc5oc6ccccc6c5c4)c23)cc1 ; c1ccc(-c2ccc3c(c2)oc2c(-c4ccccc4)ccc(N(c4ccc(-n5c6ccccc6c6ccccc65)cc4)c4ccc5c(c4)oc4ccccc45)c23)cc1]

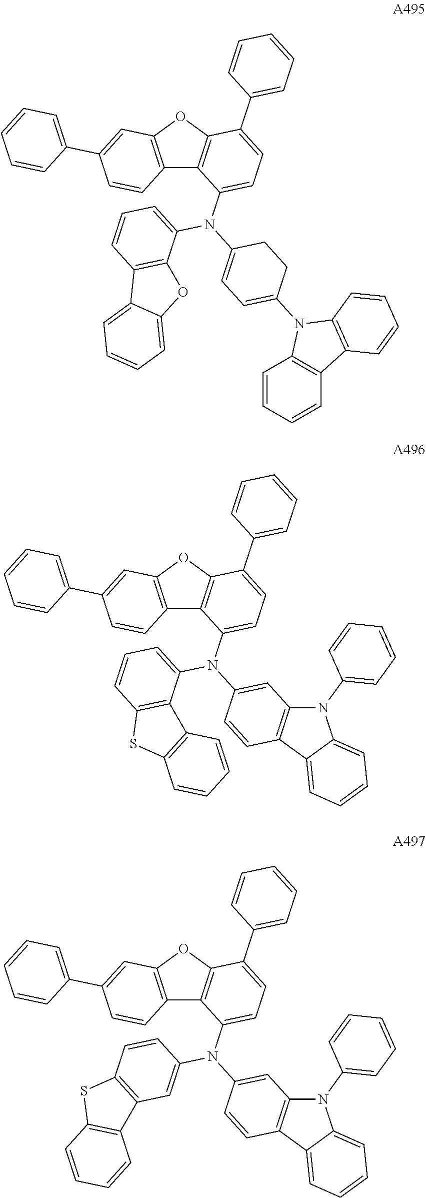 [SMILES: C1=C(N(c2cccc3c2oc2ccccc23)c2ccc(-c3ccccc3)c3oc4cc(-c5ccccc5)ccc4c23)CCC(n2c3ccccc3c3ccccc32)=C1 ; c1ccc(-c2ccc3c(c2)oc2c(-c4ccccc4)ccc(N(c4ccc5c6ccccc6n(-c6ccccc6)c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4ccc5c6ccccc6n(-c6ccccc6)c5c4)c23)cc1]

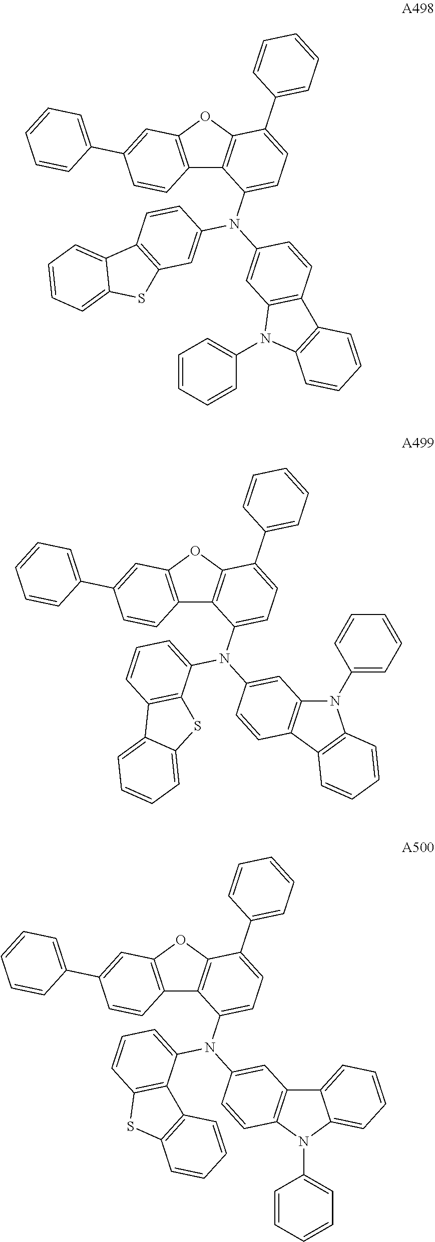 [SMILES: c1ccc(-c2ccc3c(c2)oc2c(-c4ccccc4)ccc(N(c4ccc5c(c4)sc4ccccc45)c4ccc5c6ccccc6n(-c6ccccc6)c5c4)c23)cc1 ; c1ccc(-c2ccc3c(c2)oc2c(-c4ccccc4)ccc(N(c4ccc5c6ccccc6n(-c6ccccc6)c5c4)c4cccc5c4sc4ccccc45)c23)cc1 ; c1ccc(-c2ccc3c(c2)oc2c(-c4ccccc4)ccc(N(c4ccc5c(c4)c4ccccc4n5-c4ccccc4)c4cccc5sc6ccccc6c45)c23)cc1]

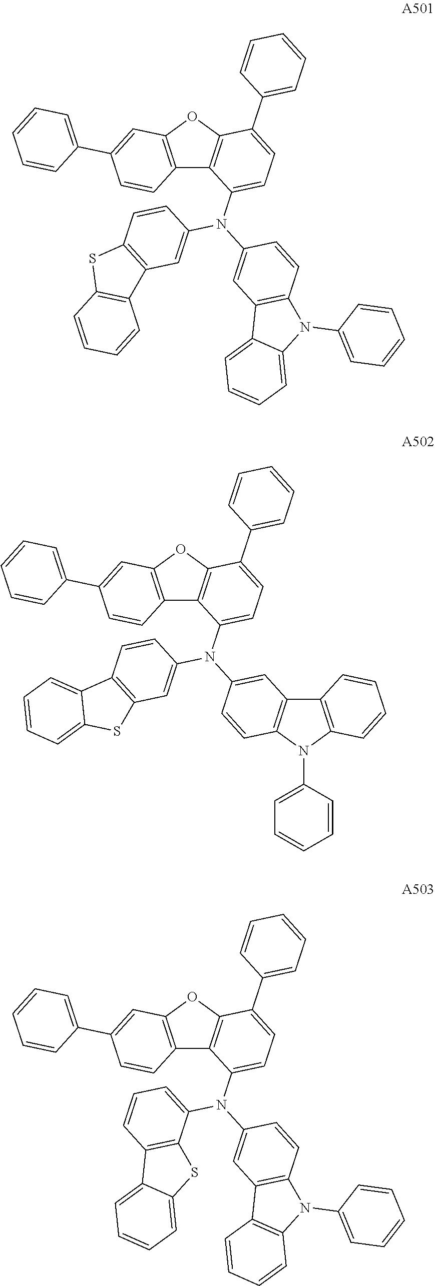 [SMILES: c1ccc(-c2ccc3c(c2)oc2c(-c4ccccc4)ccc(N(c4ccc5sc6ccccc6c5c4)c4ccc5c(c4)c4ccccc4n5-c4ccccc4)c23)cc1 ; c1ccc(-c2ccc3c(c2)oc2c(-c4ccccc4)ccc(N(c4ccc5c(c4)sc4ccccc45)c4ccc5c(c4)c4ccccc4n5-c4ccccc4)c23)cc1 ; c1ccc(-c2ccc3c(c2)oc2c(-c4ccccc4)ccc(N(c4ccc5c(c4)c4ccccc4n5-c4ccccc4)c4cccc5c4sc4ccccc45)c23)cc1]

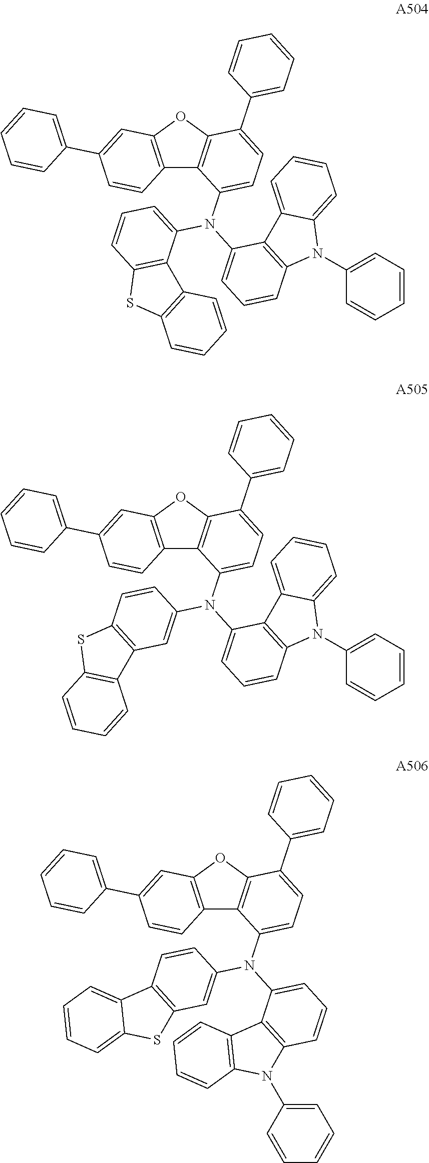 [SMILES: c1ccc(-c2ccc3c(c2)oc2c(-c4ccccc4)ccc(N(c4cccc5sc6ccccc6c45)c4cccc5c4c4ccccc4n5-c4ccccc4)c23)cc1 ; c1ccc(-c2ccc3c(c2)oc2c(-c4ccccc4)ccc(N(c4ccc5sc6ccccc6c5c4)c4cccc5c4c4ccccc4n5-c4ccccc4)c23)cc1 ; c1ccc(-c2ccc3c(c2)oc2c(-c4ccccc4)ccc(N(c4ccc5c(c4)sc4ccccc45)c4cccc5c4c4ccccc4n5-c4ccccc4)c23)cc1]

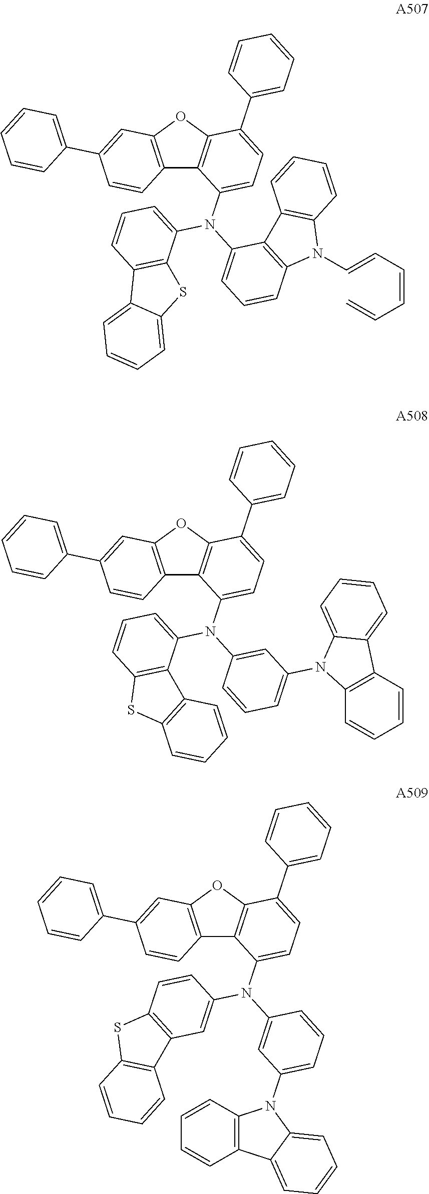 [SMILES: C=C/C=C\C=C\n1c2ccccc2c2c(N(c3cccc4c3sc3ccccc34)c3ccc(-c4ccccc4)c4oc5cc(-c6ccccc6)ccc5c34)cccc21 ; c1ccc(-c2ccc3c(c2)oc2c(-c4ccccc4)ccc(N(c4cccc(-n5c6ccccc6c6ccccc65)c4)c4cccc5sc6ccccc6c45)c23)cc1 ; c1ccc(-c2ccc3c(c2)oc2c(-c4ccccc4)ccc(N(c4cccc(-n5c6ccccc6c6ccccc65)c4)c4ccc5sc6ccccc6c5c4)c23)cc1]

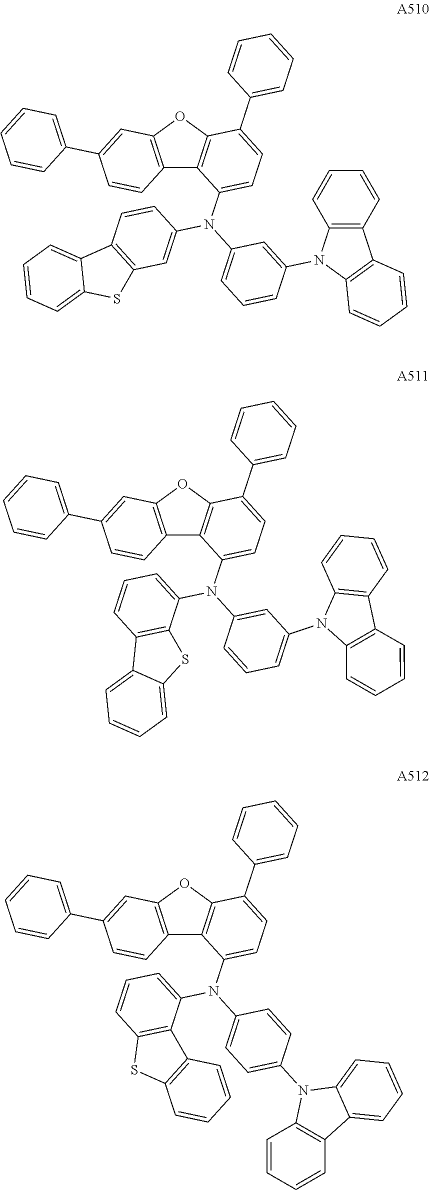 [SMILES: c1ccc(-c2ccc3c(c2)oc2c(-c4ccccc4)ccc(N(c4cccc(-n5c6ccccc6c6ccccc65)c4)c4ccc5c(c4)sc4ccccc45)c23)cc1 ; c1ccc(-c2ccc3c(c2)oc2c(-c4ccccc4)ccc(N(c4cccc(-n5c6ccccc6c6ccccc65)c4)c4cccc5c4sc4ccccc45)c23)cc1 ; c1ccc(-c2ccc3c(c2)oc2c(-c4ccccc4)ccc(N(c4ccc(-n5c6ccccc6c6ccccc65)cc4)c4cccc5sc6ccccc6c45)c23)cc1]

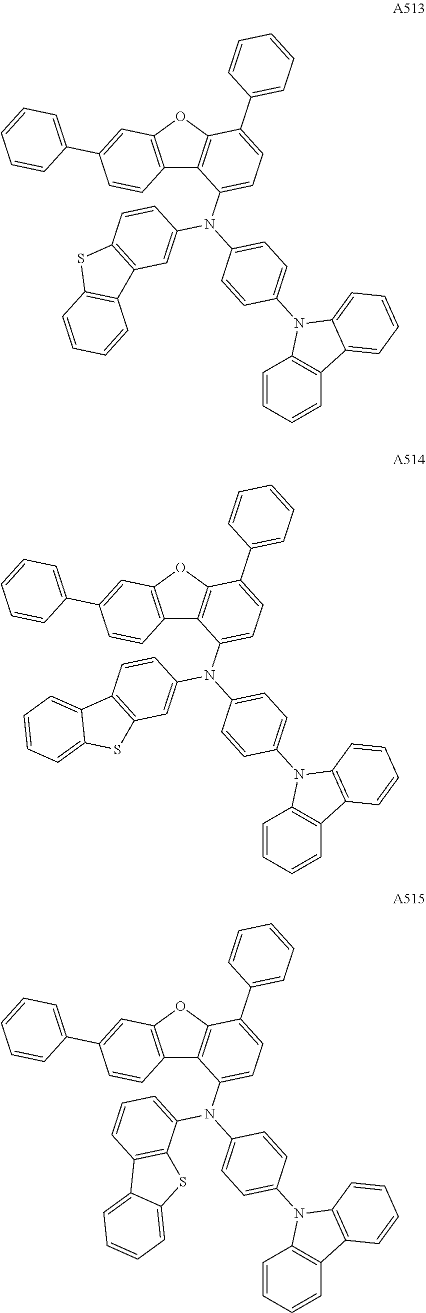 [SMILES: c1ccc(-c2ccc3c(c2)oc2c(-c4ccccc4)ccc(N(c4ccc(-n5c6ccccc6c6ccccc65)cc4)c4ccc5sc6ccccc6c5c4)c23)cc1 ; c1ccc(-c2ccc3c(c2)oc2c(-c4ccccc4)ccc(N(c4ccc(-n5c6ccccc6c6ccccc65)cc4)c4ccc5c(c4)sc4ccccc45)c23)cc1 ; c1ccc(-c2ccc3c(c2)oc2c(-c4ccccc4)ccc(N(c4ccc(-n5c6ccccc6c6ccccc65)cc4)c4cccc5c4sc4ccccc45)c23)cc1]

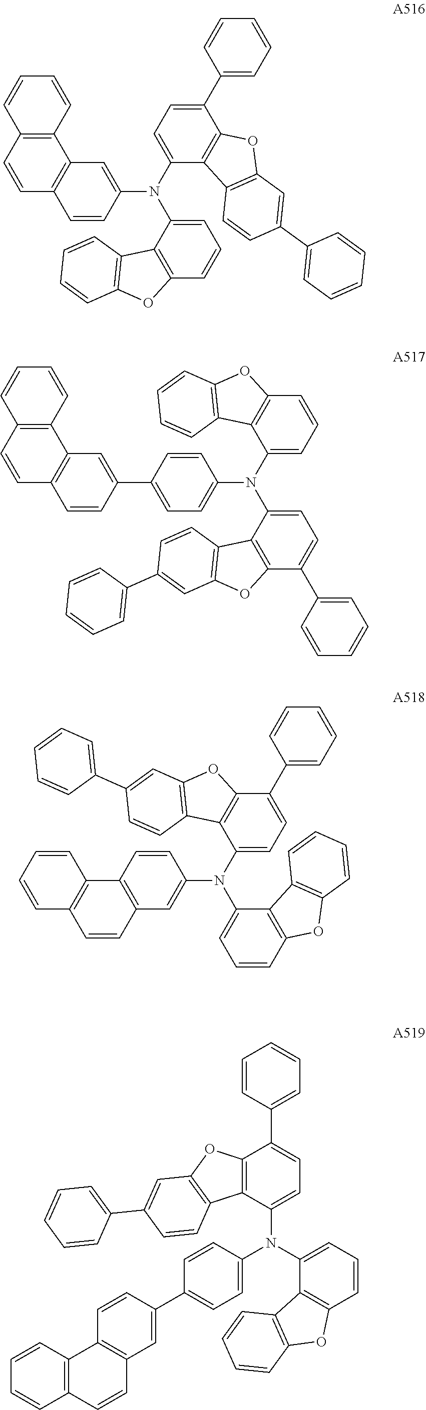 [SMILES: c1ccc(-c2ccc3c(c2)oc2c(-c4ccccc4)ccc(N(c4ccc5ccc6ccccc6c5c4)c4cccc5oc6ccccc6c45)c23)cc1 ; c1ccc(-c2ccc3c(c2)oc2c(-c4ccccc4)ccc(N(c4ccc(-c5ccc6ccc7ccccc7c6c5)cc4)c4cccc5oc6ccccc6c45)c23)cc1 ; c1ccc(-c2ccc3c(c2)oc2c(-c4ccccc4)ccc(N(c4ccc5c(ccc6ccccc65)c4)c4cccc5oc6ccccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5oc6ccccc6c45)c23)cc1]

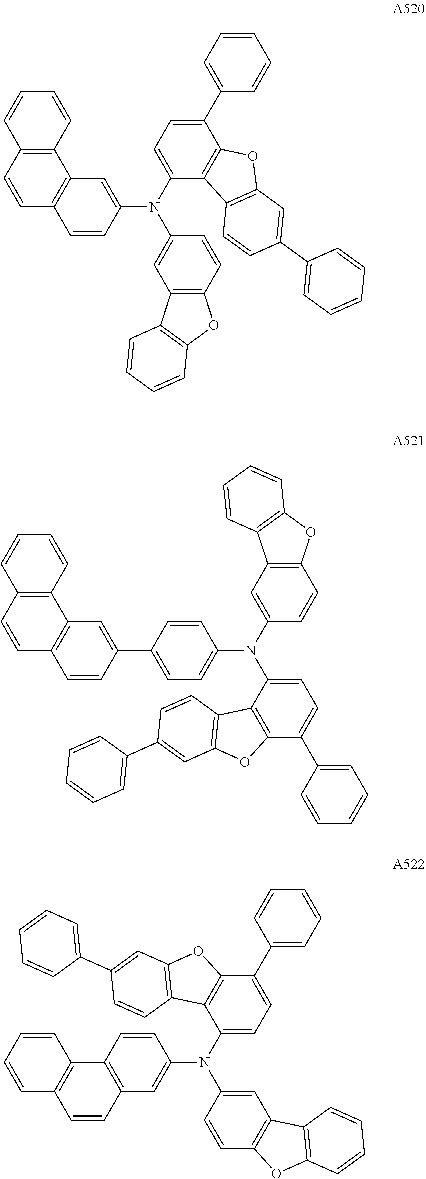 [SMILES: c1ccc(-c2ccc3c(c2)oc2c(-c4ccccc4)ccc(N(c4ccc5ccc6ccccc6c5c4)c4ccc5oc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5oc6ccccc6c5c4)c23)cc1 ; c1ccc(-c2ccc3c(c2)oc2c(-c4ccccc4)ccc(N(c4ccc5c(ccc6ccccc65)c4)c4ccc5oc6ccccc6c5c4)c23)cc1]

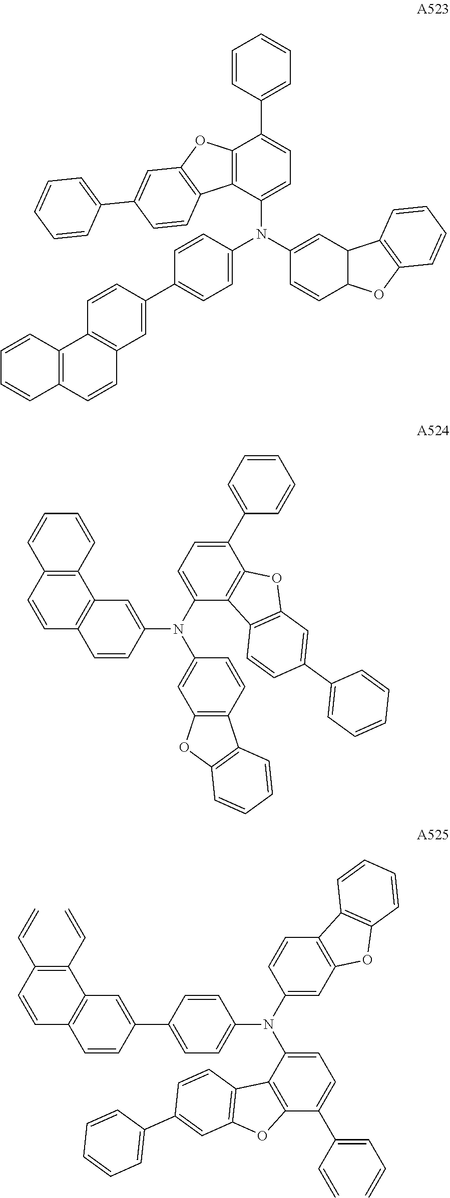 [SMILES: C1=CC2Oc3ccccc3C2C=C1N(c1ccc(-c2ccc3c(ccc4ccccc43)c2)cc1)c1ccc(-c2ccccc2)c2oc3cc(-c4ccccc4)ccc3c12 ; c1ccc(-c2ccc3c(c2)oc2c(-c4ccccc4)ccc(N(c4ccc5c(c4)oc4ccccc45)c4ccc5ccc6ccccc6c5c4)c23)cc1 ; C=C/C=C(\C=C)c1ccc(N(c2ccc(-c3ccc4ccc(C=C)c(C=C)c4c3)cc2)c2ccc3c(c2)oc2ccccc23)c2c1oc1cc(-c3ccccc3)ccc12]

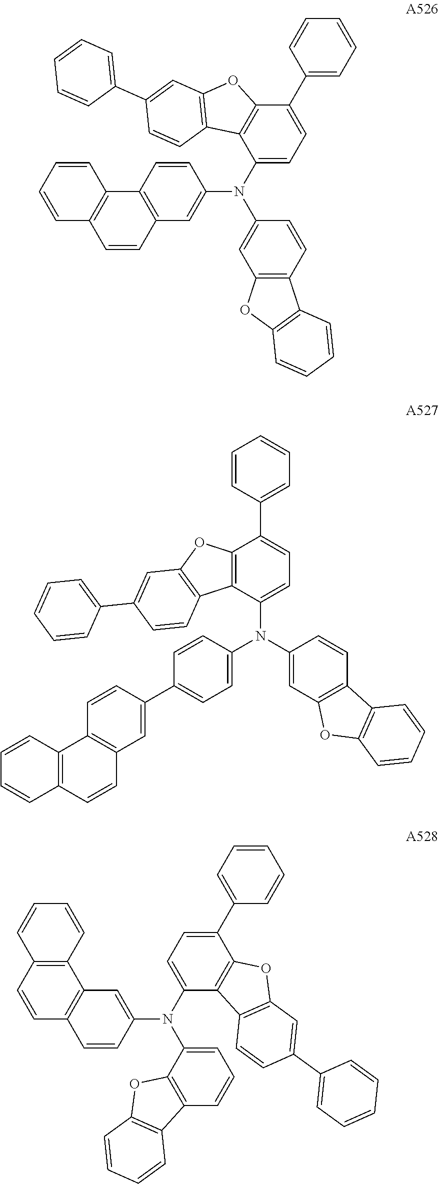 [SMILES: c1ccc(-c2ccc3c(c2)oc2c(-c4ccccc4)ccc(N(c4ccc5c(ccc6ccccc65)c4)c4ccc5c(c4)oc4ccccc45)c23)cc1 ; c1ccc(-c2ccc3c(c2)oc2c(-c4ccccc4)ccc(N(c4ccc(-c5ccc6c(ccc7ccccc76)c5)cc4)c4ccc5c(c4)oc4ccccc45)c23)cc1 ; c1ccc(-c2ccc3c(c2)oc2c(-c4ccccc4)ccc(N(c4ccc5ccc6ccccc6c5c4)c4cccc5c4oc4ccccc45)c23)cc1]

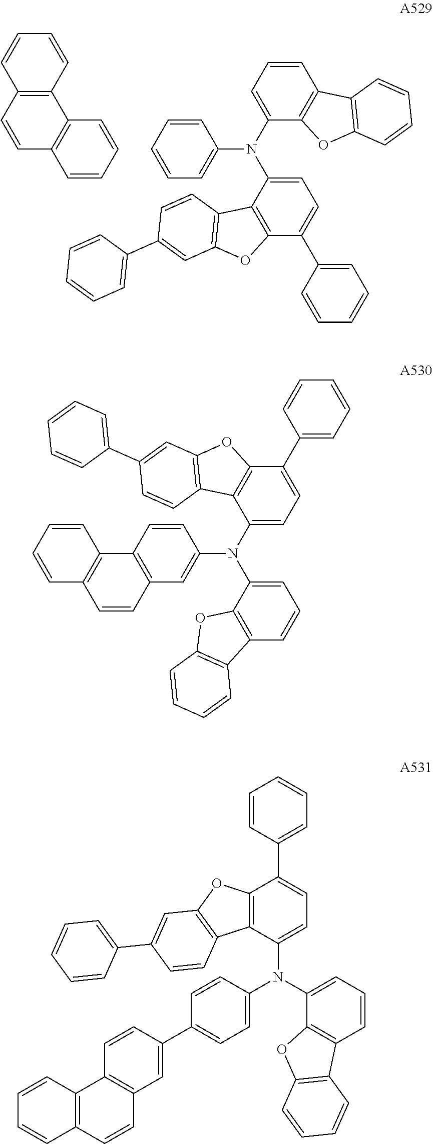

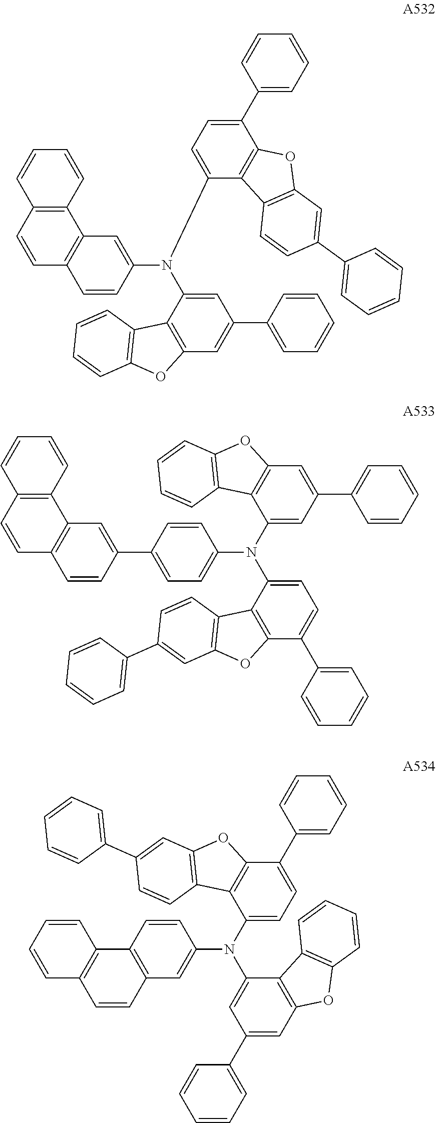 [SMILES: c1ccc(-c2ccc3c(c2)oc2c(-c4ccccc4)ccc(N(c4ccc5ccc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc(-c5ccc6ccc7ccccc7c6c5)cc4)c4cc(-c5ccccc5)cc5oc6ccccc6c45)c23)cc1 ; c1ccc(-c2ccc3c(c2)oc2c(-c4ccccc4)ccc(N(c4ccc5c(ccc6ccccc65)c4)c4cc(-c5ccccc5)cc5oc6ccccc6c45)c23)cc1]

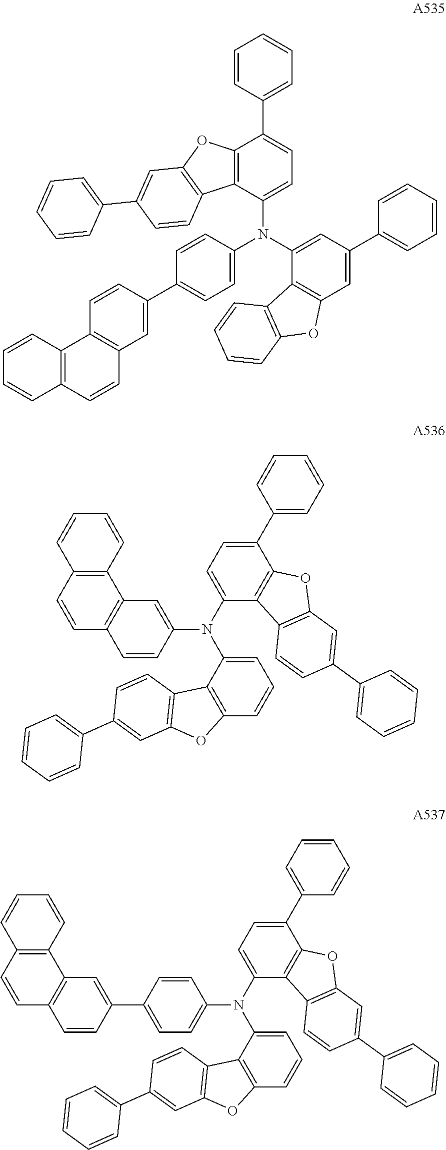 [SMILES: c1ccc(-c2ccc3c(c2)oc2c(-c4ccccc4)ccc(N(c4ccc(-c5ccc6c(ccc7ccccc76)c5)cc4)c4cc(-c5ccccc5)cc5oc6ccccc6c45)c23)cc1 ; c1ccc(-c2ccc3c(c2)oc2cccc(N(c4ccc5ccc6ccccc6c5c4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5oc6cc(-c7ccccc7)ccc6c45)c23)cc1]

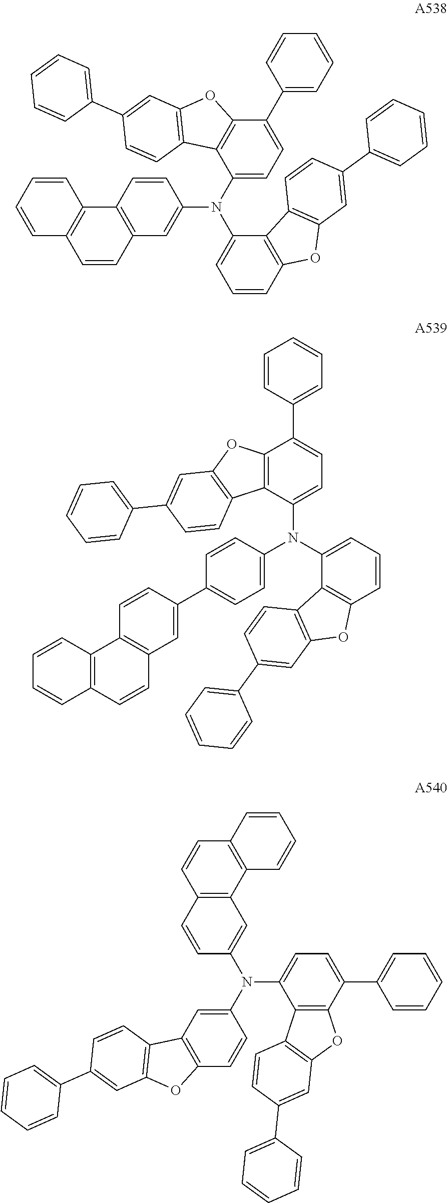 [SMILES: c1ccc(-c2ccc3c(c2)oc2cccc(N(c4ccc5c(ccc6ccccc65)c4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5oc6cc(-c7ccccc7)ccc6c45)c23)cc1 ; c1ccc(-c2ccc3c(c2)oc2ccc(N(c4ccc5ccc6ccccc6c5c4)c4ccc(-c5ccccc5)c5oc6cc(-c7ccccc7)ccc6c45)cc23)cc1]

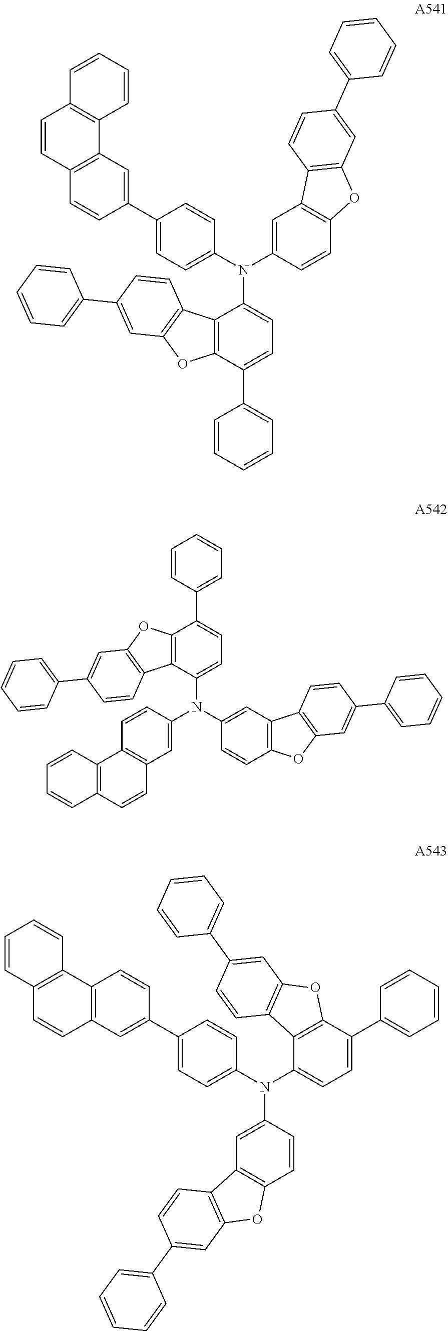 [SMILES: c1ccc(-c2ccc3c(c2)oc2ccc(N(c4ccc(-c5ccc6ccc7ccccc7c6c5)cc4)c4ccc(-c5ccccc5)c5oc6cc(-c7ccccc7)ccc6c45)cc23)cc1 ; c1ccc(-c2ccc3c(c2)oc2ccc(N(c4ccc5c(ccc6ccccc65)c4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6c(ccc7ccccc76)c5)cc4)c4ccc(-c5ccccc5)c5oc6cc(-c7ccccc7)ccc6c45)cc23)cc1]

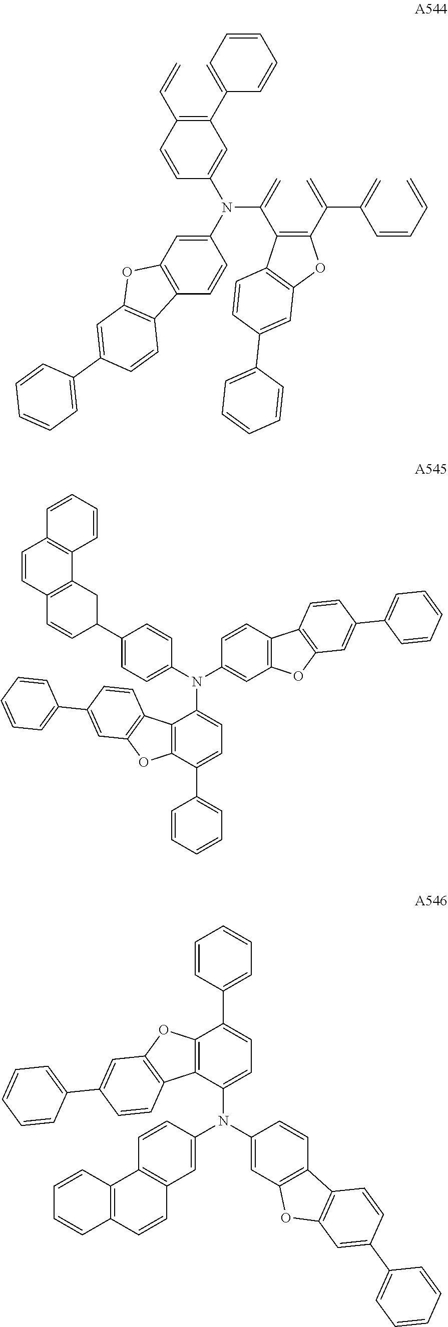 [SMILES: C=C/C=C\C(=C)C(=C)c1oc2cc(-c3ccccc3)ccc2c1C(=C)N(c1ccc(C=C)c(-c2ccccc2)c1)c1ccc2c(c1)oc1cc(-c3ccccc3)ccc12 ; C1=CC(c2ccc(N(c3ccc4c(c3)oc3cc(-c5ccccc5)ccc34)c3ccc(-c4ccccc4)c4oc5cc(-c6ccccc6)ccc5c34)cc2)Cc2c1ccc1ccccc21 ; c1ccc(-c2ccc3c(c2)oc2cc(N(c4ccc5c(ccc6ccccc65)c4)c4ccc(-c5ccccc5)c5oc6cc(-c7ccccc7)ccc6c45)ccc23)cc1]

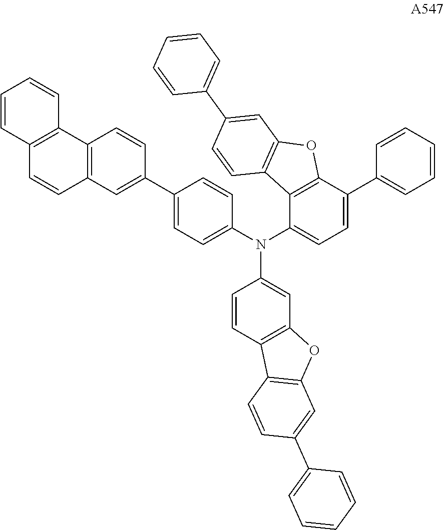 [SMILES: c1ccc(-c2ccc3c(c2)oc2cc(N(c4ccc(-c5ccc6c(ccc7ccccc76)c5)cc4)c4ccc(-c5ccccc5)c5oc6cc(-c7ccccc7)ccc6c45)ccc23)cc1]

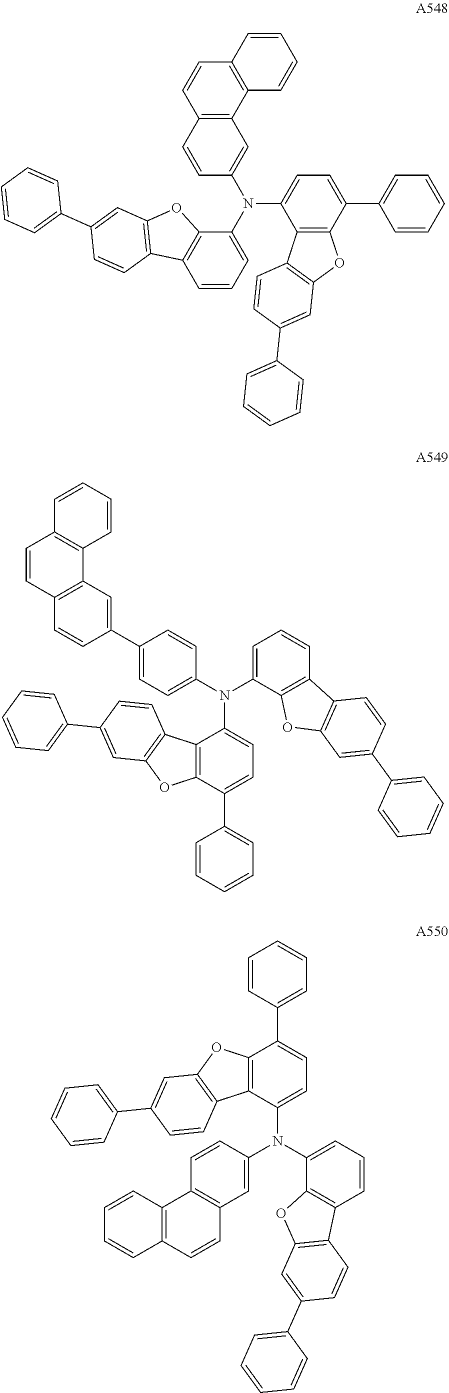 [SMILES: c1ccc(-c2ccc3c(c2)oc2c(N(c4ccc5ccc6ccccc6c5c4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6ccc7ccccc7c6c5)cc4)c4ccc(-c5ccccc5)c5oc6cc(-c7ccccc7)ccc6c45)cccc23)cc1 ; c1ccc(-c2ccc3c(c2)oc2c(N(c4ccc5c(ccc6ccccc65)c4)c4ccc(-c5ccccc5)c5oc6cc(-c7ccccc7)ccc6c45)cccc23)cc1]

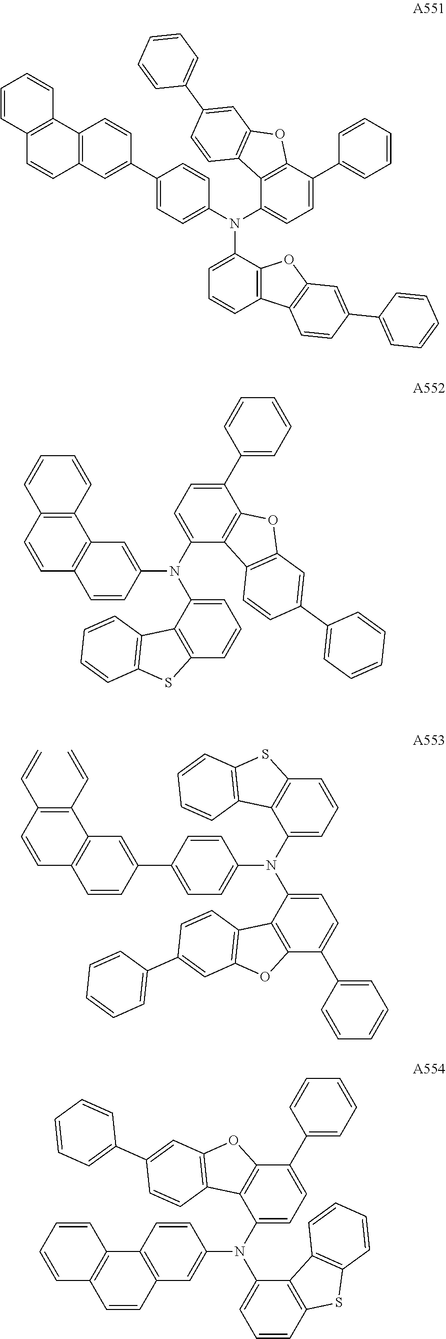

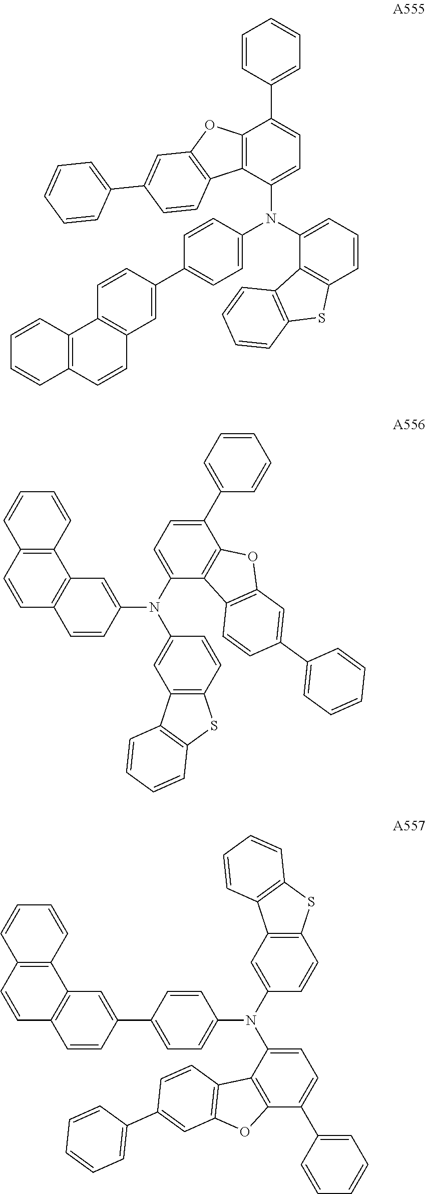 [SMILES: c1ccc(-c2ccc3c(c2)oc2c(-c4ccccc4)ccc(N(c4ccc(-c5ccc6c(ccc7ccccc76)c5)cc4)c4cccc5sc6ccccc6c45)c23)cc1 ; c1ccc(-c2ccc3c(c2)oc2c(-c4ccccc4)ccc(N(c4ccc5ccc6ccccc6c5c4)c4ccc5sc6ccccc6c5c4)c23)cc1 ; c1ccc(-c2ccc3c(c2)oc2c(-c4ccccc4)ccc(N(c4ccc(-c5ccc6ccc7ccccc7c6c5)cc4)c4ccc5sc6ccccc6c5c4)c23)cc1]

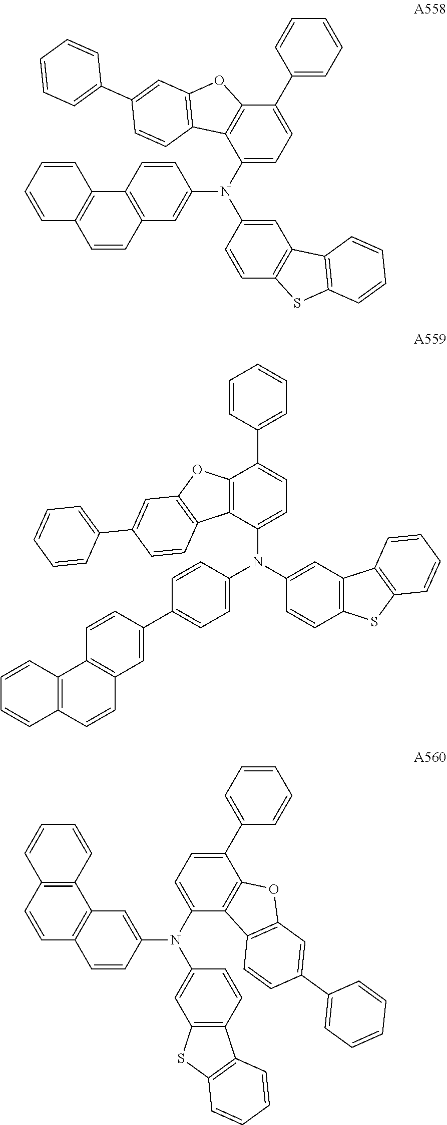 [SMILES: c1ccc(-c2ccc3c(c2)oc2c(-c4ccccc4)ccc(N(c4ccc5c(ccc6ccccc65)c4)c4ccc5sc6ccccc6c5c4)c23)cc1 ; c1ccc(-c2ccc3c(c2)oc2c(-c4ccccc4)ccc(N(c4ccc(-c5ccc6c(ccc7ccccc76)c5)cc4)c4ccc5sc6ccccc6c5c4)c23)cc1 ; c1ccc(-c2ccc3c(c2)oc2c(-c4ccccc4)ccc(N(c4ccc5c(c4)sc4ccccc45)c4ccc5ccc6ccccc6c5c4)c23)cc1]

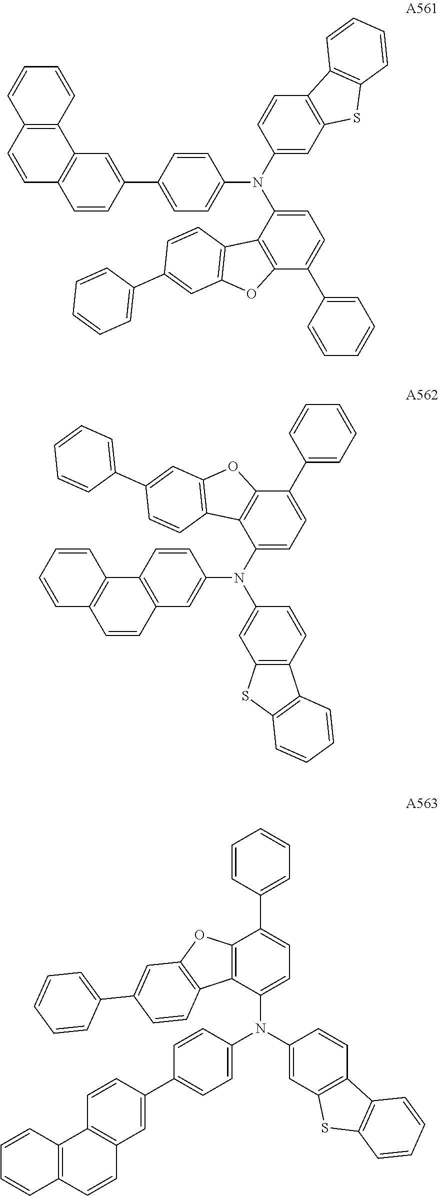 [SMILES: c1ccc(-c2ccc3c(c2)oc2c(-c4ccccc4)ccc(N(c4ccc(-c5ccc6ccc7ccccc7c6c5)cc4)c4ccc5c(c4)sc4ccccc45)c23)cc1 ; c1ccc(-c2ccc3c(c2)oc2c(-c4ccccc4)ccc(N(c4ccc5c(ccc6ccccc65)c4)c4ccc5c(c4)sc4ccccc45)c23)cc1 ; c1ccc(-c2ccc3c(c2)oc2c(-c4ccccc4)ccc(N(c4ccc(-c5ccc6c(ccc7ccccc76)c5)cc4)c4ccc5c(c4)sc4ccccc45)c23)cc1]

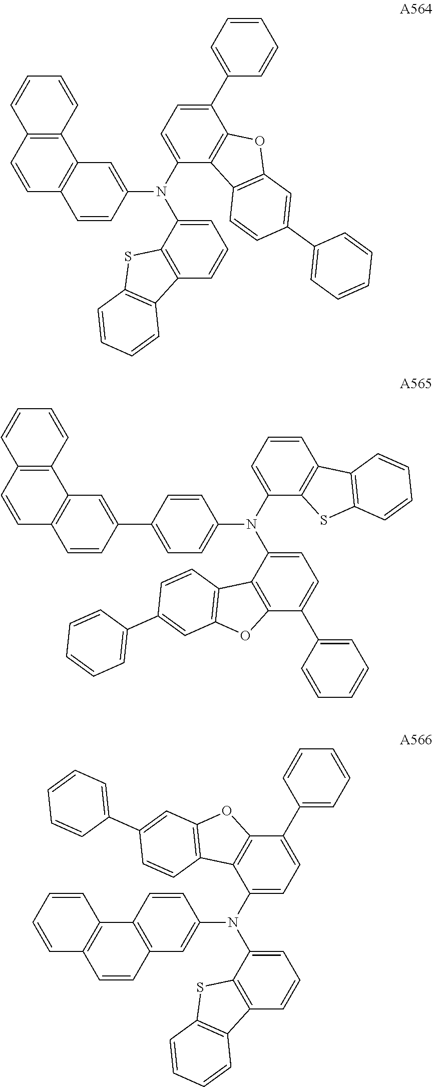 [SMILES: c1ccc(-c2ccc3c(c2)oc2c(-c4ccccc4)ccc(N(c4ccc5ccc6ccccc6c5c4)c4cccc5c4sc4ccccc45)c23)cc1 ; c1ccc(-c2ccc3c(c2)oc2c(-c4ccccc4)ccc(N(c4ccc(-c5ccc6ccc7ccccc7c6c5)cc4)c4cccc5c4sc4ccccc45)c23)cc1 ; c1ccc(-c2ccc3c(c2)oc2c(-c4ccccc4)ccc(N(c4ccc5c(ccc6ccccc65)c4)c4cccc5c4sc4ccccc45)c23)cc1]

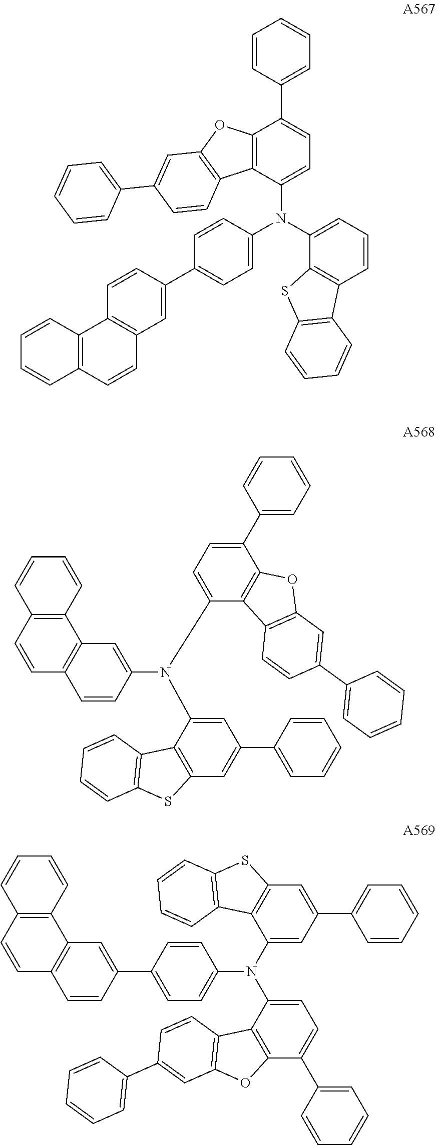 [SMILES: c1ccc(-c2ccc3c(c2)oc2c(-c4ccccc4)ccc(N(c4ccc(-c5ccc6c(ccc7ccccc76)c5)cc4)c4cccc5c4sc4ccccc45)c23)cc1 ; c1ccc(-c2ccc3c(c2)oc2c(-c4ccccc4)ccc(N(c4ccc5ccc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc(-c5ccc6ccc7ccccc7c6c5)cc4)c4cc(-c5ccccc5)cc5sc6ccccc6c45)c23)cc1]

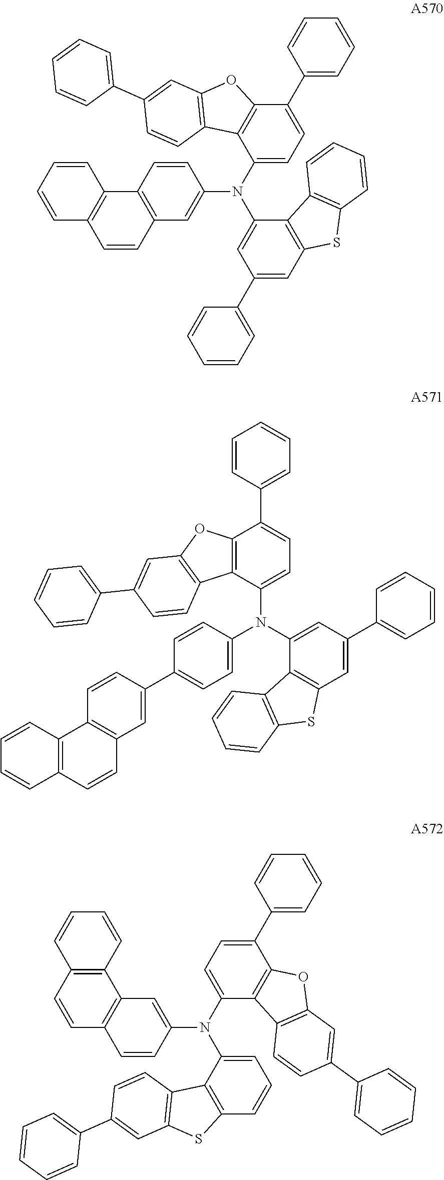 [SMILES: c1ccc(-c2ccc3c(c2)oc2c(-c4ccccc4)ccc(N(c4ccc5c(ccc6ccccc65)c4)c4cc(-c5ccccc5)cc5sc6ccccc6c45)c23)cc1 ; c1ccc(-c2ccc3c(c2)oc2c(-c4ccccc4)ccc(N(c4ccc(-c5ccc6c(ccc7ccccc76)c5)cc4)c4cc(-c5ccccc5)cc5sc6ccccc6c45)c23)cc1 ; c1ccc(-c2ccc3c(c2)oc2c(-c4ccccc4)ccc(N(c4ccc5ccc6ccccc6c5c4)c4cccc5sc6cc(-c7ccccc7)ccc6c45)c23)cc1]

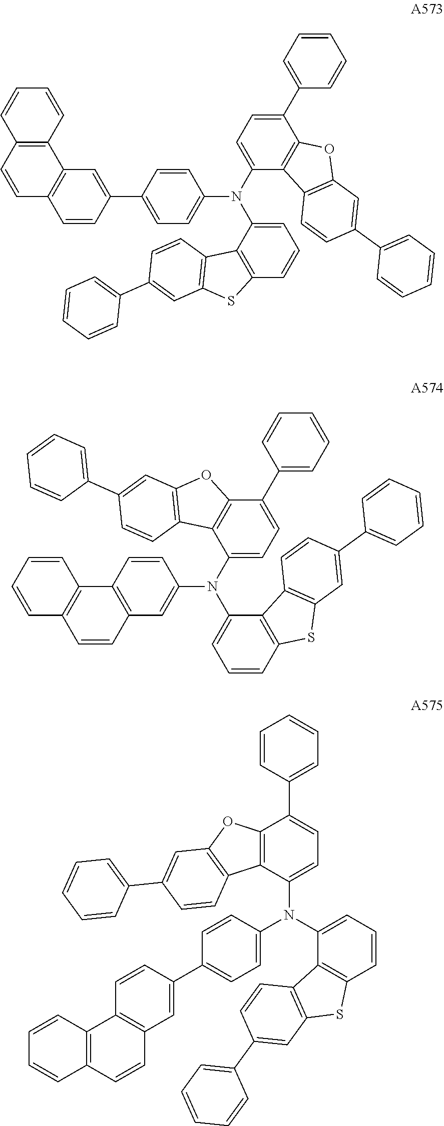 [SMILES: c1ccc(-c2ccc3c(c2)oc2c(-c4ccccc4)ccc(N(c4ccc(-c5ccc6ccc7ccccc7c6c5)cc4)c4cccc5sc6cc(-c7ccccc7)ccc6c45)c23)cc1 ; c1ccc(-c2ccc3c(c2)oc2c(-c4ccccc4)ccc(N(c4ccc5c(ccc6ccccc65)c4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6c(ccc7ccccc76)c5)cc4)c4cccc5sc6cc(-c7ccccc7)ccc6c45)c23)cc1]

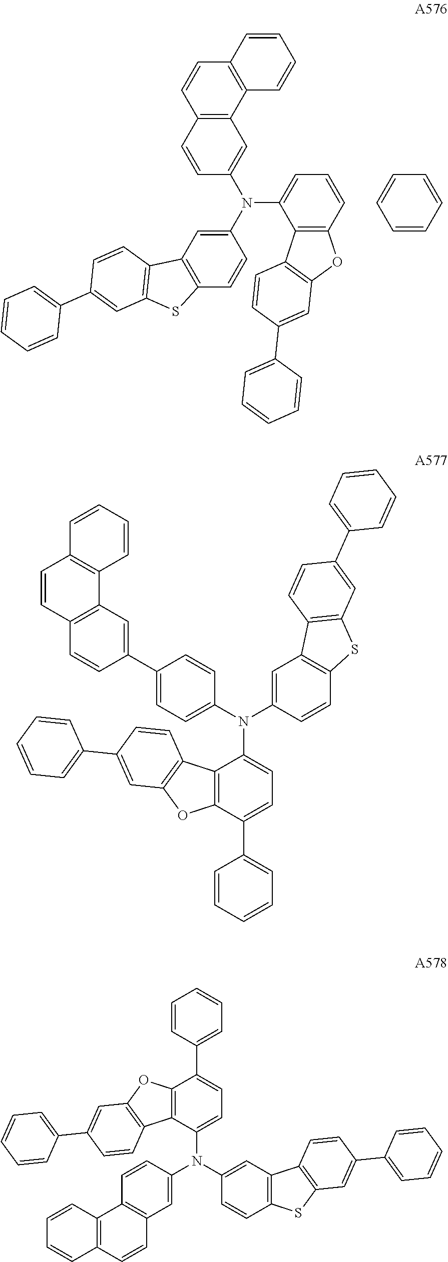

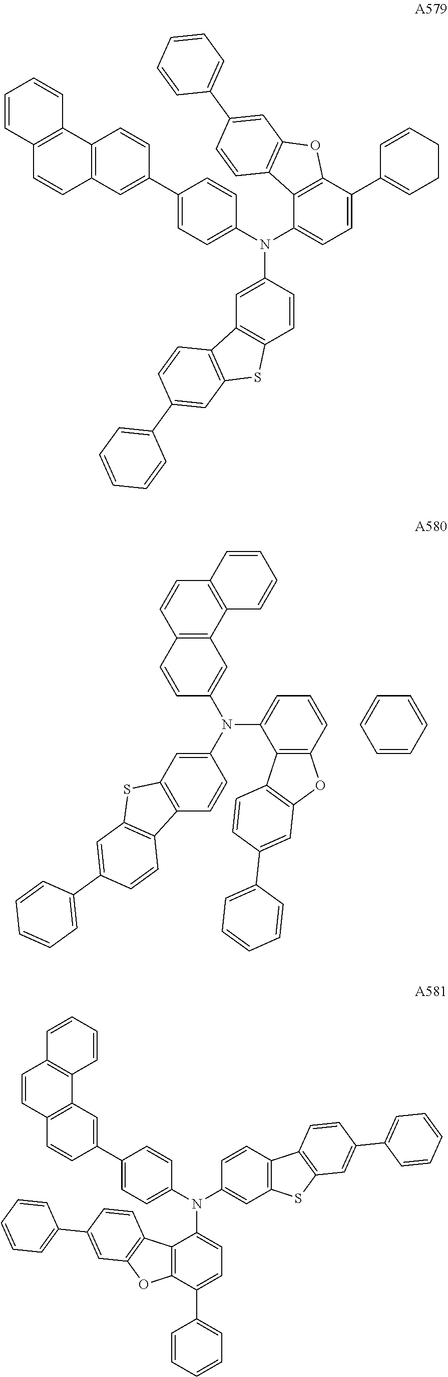

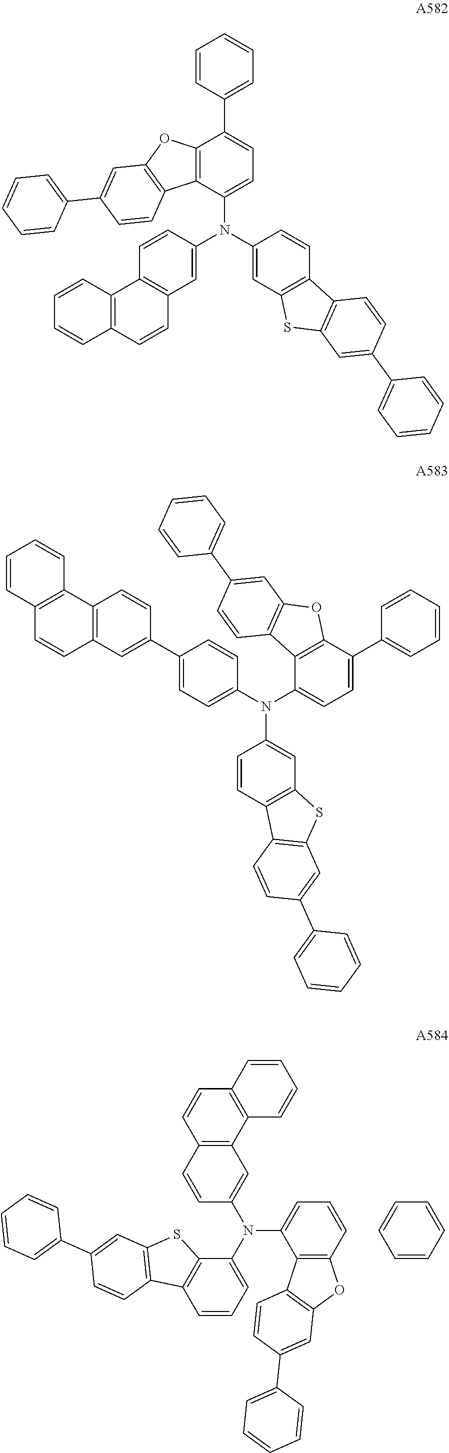

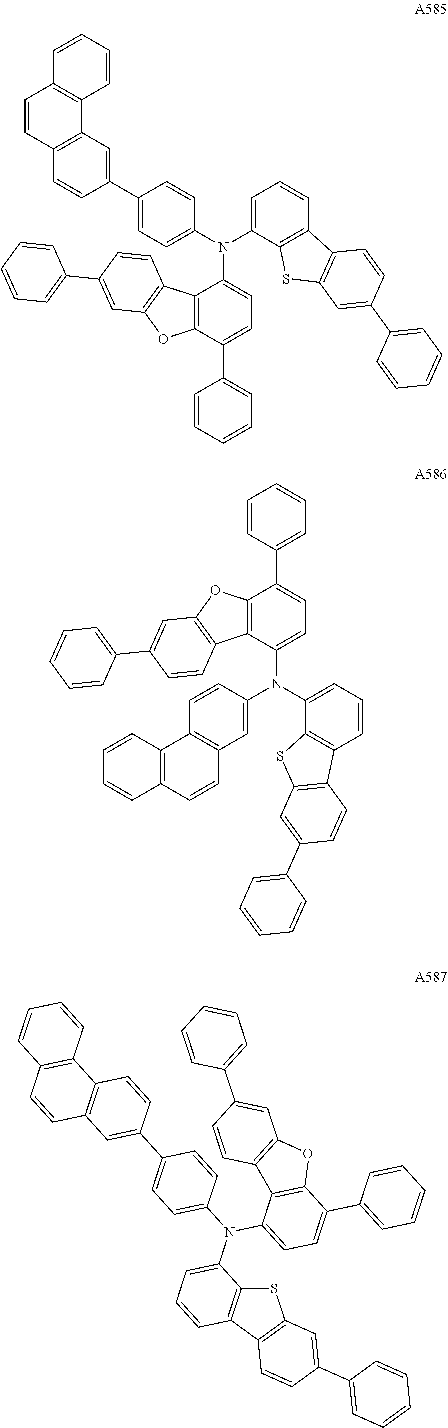 [SMILES: c1ccc(-c2ccc3c(c2)oc2c(-c4ccccc4)ccc(N(c4ccc(-c5ccc6ccc7ccccc7c6c5)cc4)c4cccc5c4sc4cc(-c6ccccc6)ccc45)c23)cc1 ; c1ccc(-c2ccc3c(c2)oc2c(-c4ccccc4)ccc(N(c4ccc5c(ccc6ccccc65)c4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6c(ccc7ccccc76)c5)cc4)c4cccc5c4sc4cc(-c6ccccc6)ccc45)c23)cc1]

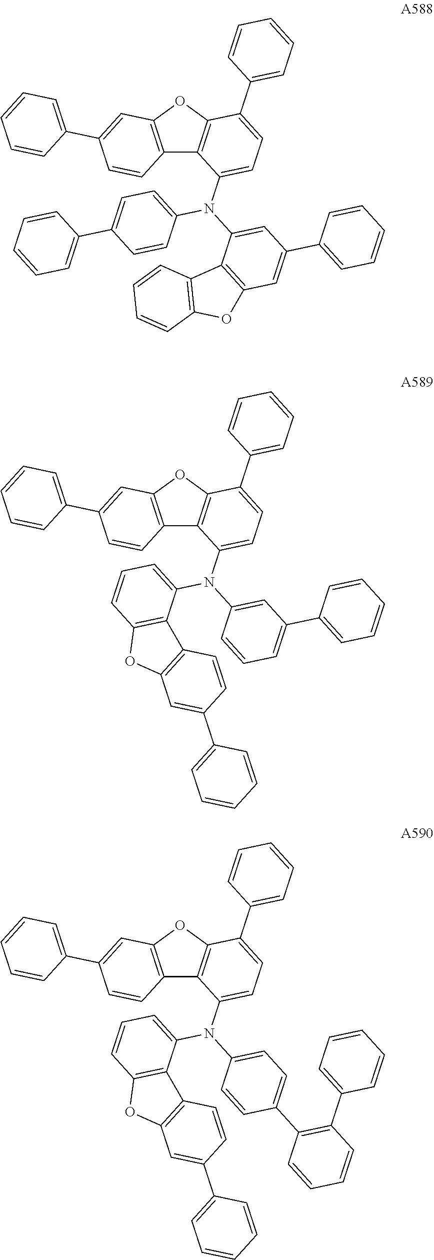

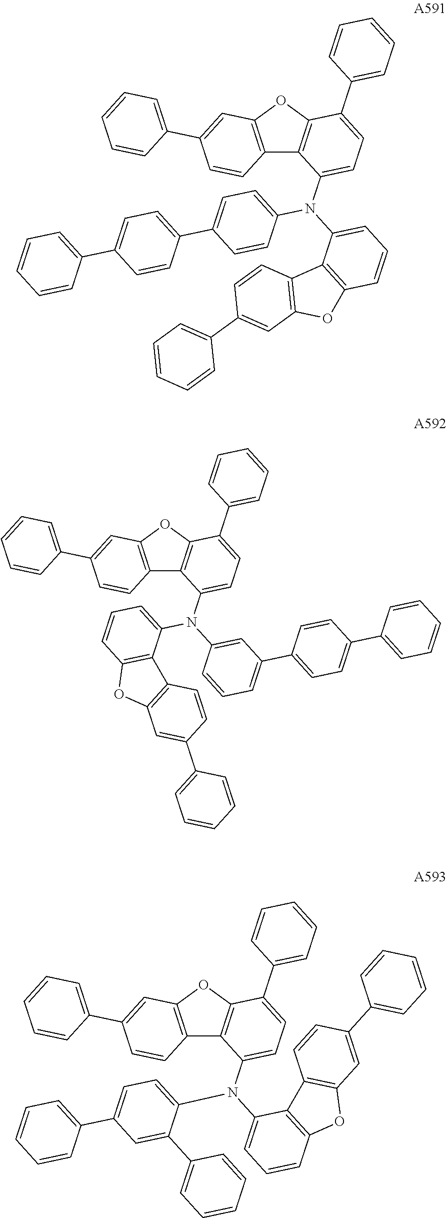 [SMILES: c1ccc(-c2ccc(-c3ccc(N(c4cccc5oc6cc(-c7ccccc7)ccc6c45)c4ccc(-c5ccccc5)c5oc6cc(-c7ccccc7)ccc6c45)cc3)cc2)cc1 ; c1ccc(-c2ccc(-c3cccc(N(c4cccc5oc6cc(-c7ccccc7)ccc6c45)c4ccc(-c5ccccc5)c5oc6cc(-c7ccccc7)ccc6c45)c3)cc2)cc1 ; c1ccc(-c2ccc(N(c3cccc4oc5cc(-c6ccccc6)ccc5c34)c3ccc(-c4ccccc4)c4oc5cc(-c6ccccc6)ccc5c34)c(-c3ccccc3)c2)cc1]

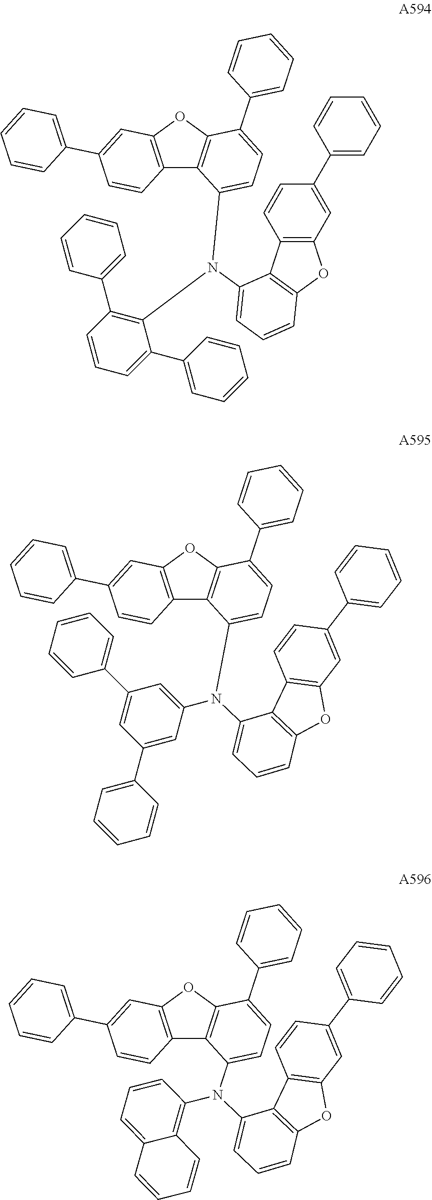 [SMILES: c1ccc(-c2ccc3c(c2)oc2cccc(N(c4c(-c5ccccc5)cccc4-c4ccccc4)c4ccc(-c5ccccc5)c5oc6cc(-c7ccccc7)ccc6c45)c23)cc1 ; c1ccc(-c2cc(-c3ccccc3)cc(N(c3cccc4oc5cc(-c6ccccc6)ccc5c34)c3ccc(-c4ccccc4)c4oc5cc(-c6ccccc6)ccc5c34)c2)cc1 ; c1ccc(-c2ccc3c(c2)oc2cccc(N(c4cccc5ccccc45)c4ccc(-c5ccccc5)c5oc6cc(-c7ccccc7)ccc6c45)c23)cc1]

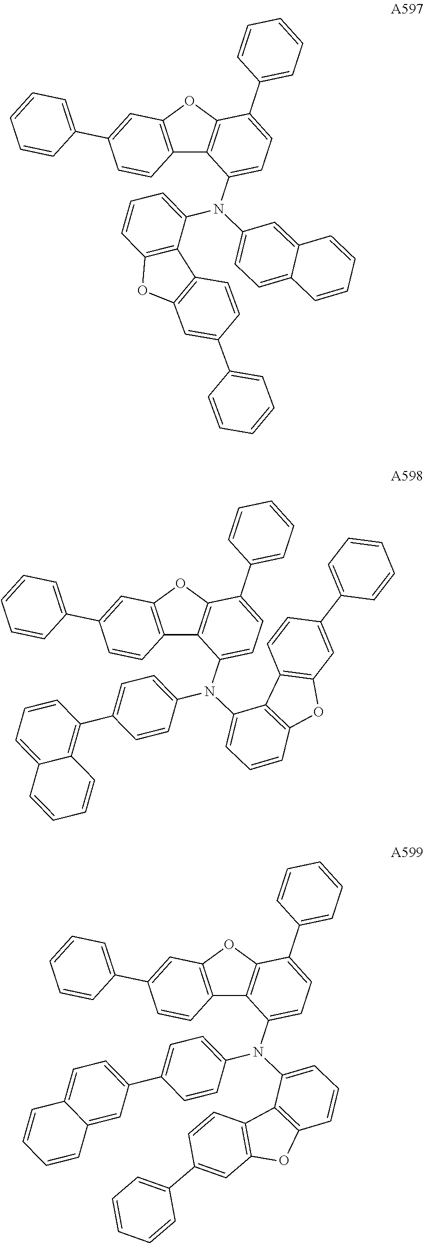 [SMILES: c1ccc(-c2ccc3c(c2)oc2cccc(N(c4ccc5ccccc5c4)c4ccc(-c5ccccc5)c5oc6cc(-c7ccccc7)ccc6c45)c23)cc1 ; c1ccc(-c2ccc3c(c2)oc2cccc(N(c4ccc(-c5cccc6ccccc56)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc6ccccc6c5)cc4)c4ccc(-c5ccccc5)c5oc6cc(-c7ccccc7)ccc6c45)c23)cc1]

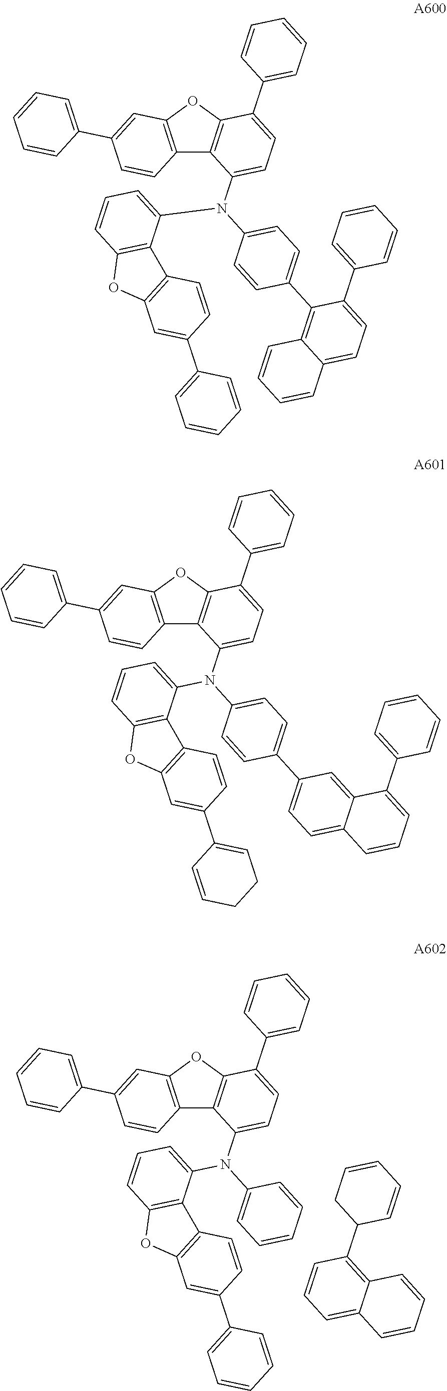

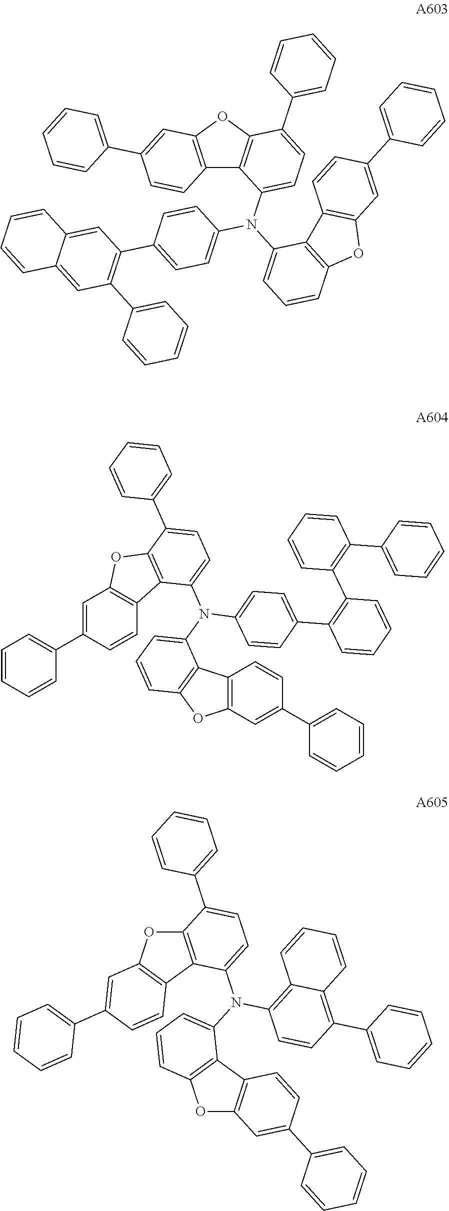 [SMILES: c1ccc(-c2ccc3c(c2)oc2cccc(N(c4ccc(-c5cc6ccccc6cc5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccccc5-c5ccccc5-c5ccccc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccccc5)c5ccccc45)c4ccc(-c5ccccc5)c5oc6cc(-c7ccccc7)ccc6c45)c23)cc1]

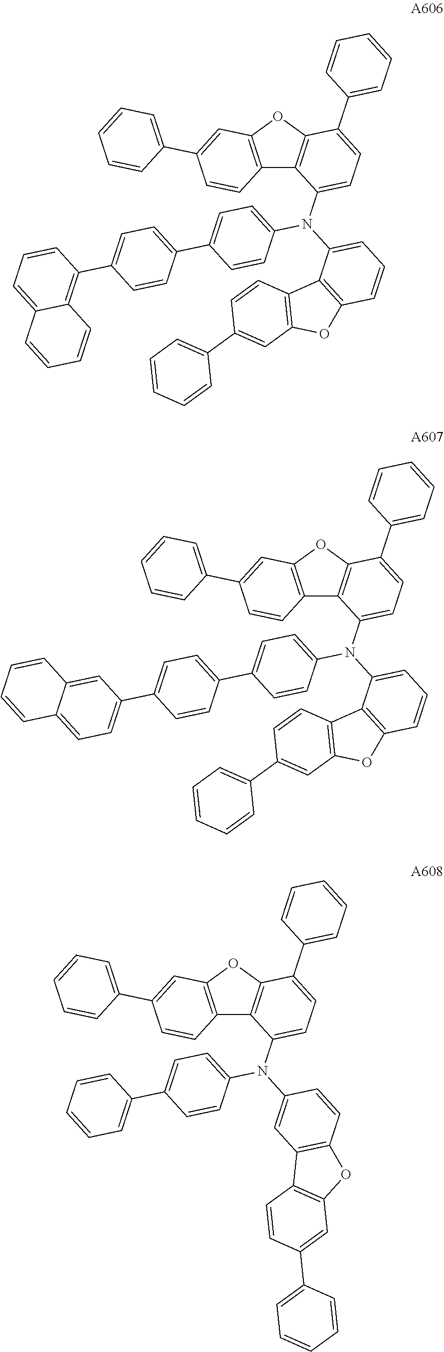 [SMILES: c1ccc(-c2ccc3c(c2)oc2cccc(N(c4ccc(-c5ccc(-c6cccc7ccccc67)cc5)cc4)c4ccc(-c5ccccc5)c5oc6cc(-c7ccccc7)ccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5oc6cc(-c7ccccc7)ccc6c45)c23)cc1 ; c1ccc(-c2ccc(N(c3ccc4oc5cc(-c6ccccc6)ccc5c4c3)c3ccc(-c4ccccc4)c4oc5cc(-c6ccccc6)ccc5c34)cc2)cc1]

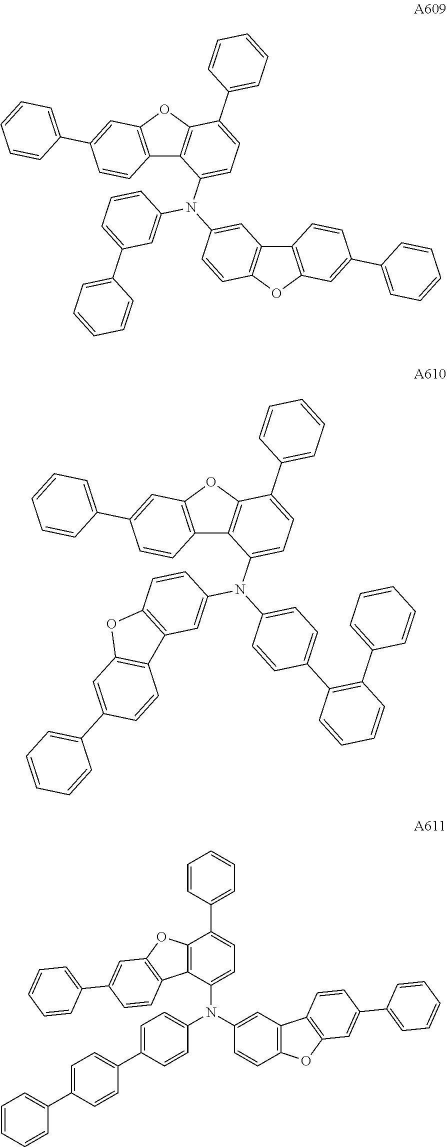 [SMILES: c1ccc(-c2cccc(N(c3ccc4oc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2ccc(N(c4ccc(-c5ccccc5-c5ccccc5)cc4)c4ccc(-c5ccccc5)c5oc6cc(-c7ccccc7)ccc6c45)cc23)cc1 ; c1ccc(-c2ccc(-c3ccc(N(c4ccc5oc6cc(-c7ccccc7)ccc6c5c4)c4ccc(-c5ccccc5)c5oc6cc(-c7ccccc7)ccc6c45)cc3)cc2)cc1]

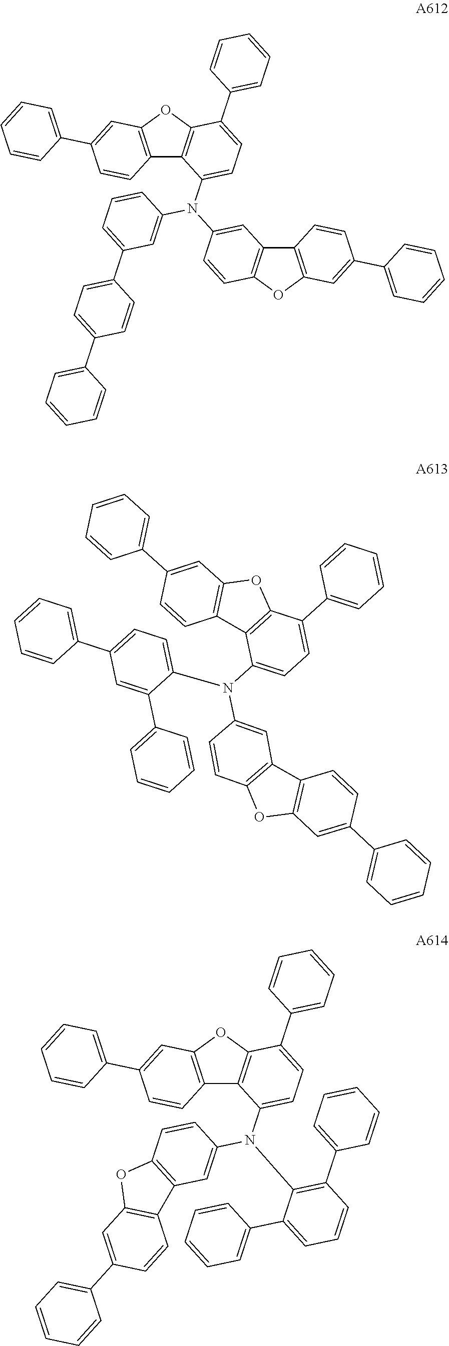 [SMILES: c1ccc(-c2ccc(-c3cccc(N(c4ccc5oc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4oc5cc(-c6ccccc6)ccc5c4c3)c3ccc(-c4ccccc4)c4oc5cc(-c6ccccc6)ccc5c34)c(-c3ccccc3)c2)cc1 ; c1ccc(-c2ccc3c(c2)oc2ccc(N(c4c(-c5ccccc5)cccc4-c4ccccc4)c4ccc(-c5ccccc5)c5oc6cc(-c7ccccc7)ccc6c45)cc23)cc1]

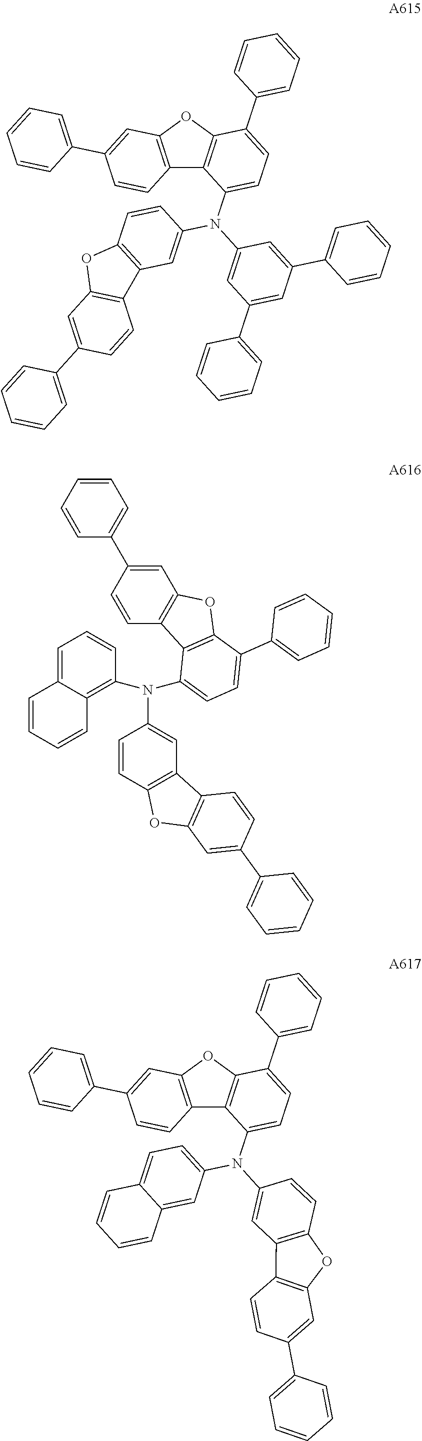

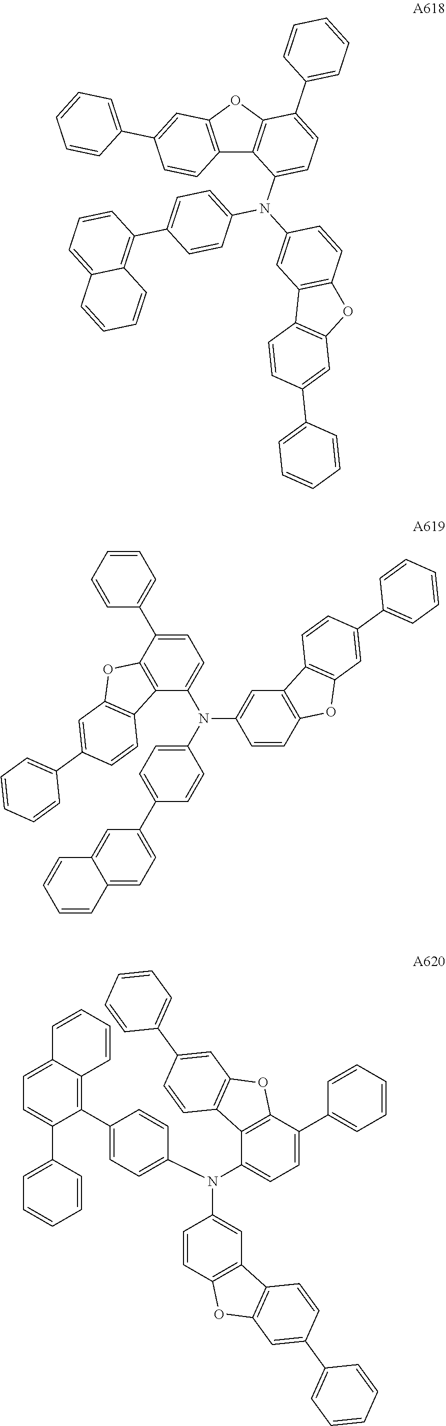 [SMILES: c1ccc(-c2ccc3c(c2)oc2ccc(N(c4ccc(-c5cccc6ccccc56)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6ccccc6c5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5c(-c6ccccc6)ccc6ccccc56)cc4)c4ccc(-c5ccccc5)c5oc6cc(-c7ccccc7)ccc6c45)cc23)cc1]

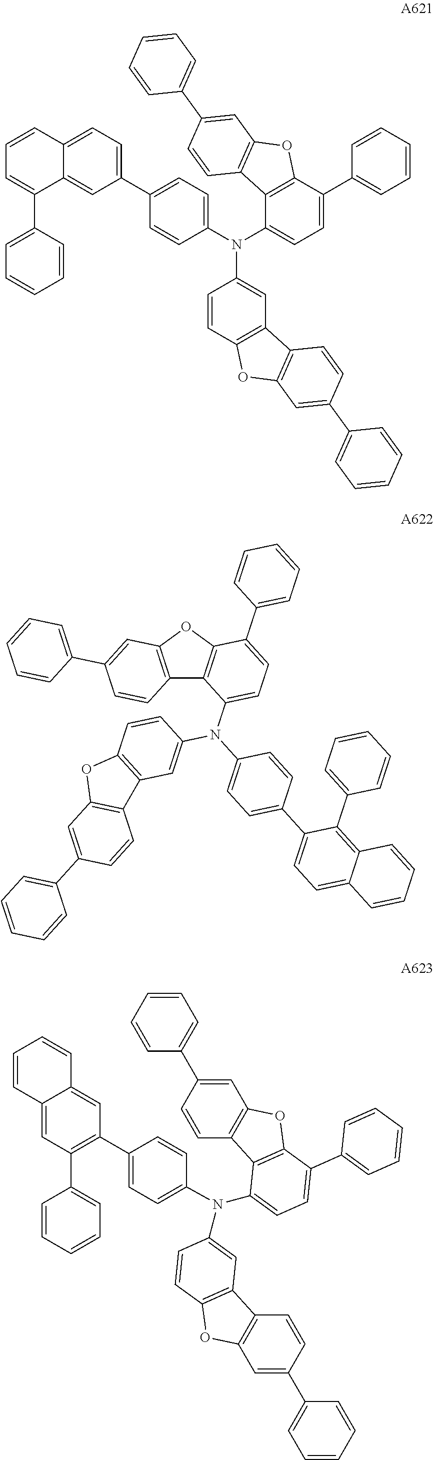 [SMILES: c1ccc(-c2ccc3c(c2)oc2ccc(N(c4ccc(-c5ccc6cccc(-c7ccccc7)c6c5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccc6ccccc6c5-c5ccccc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5cc6ccccc6cc5-c5ccccc5)cc4)c4ccc(-c5ccccc5)c5oc6cc(-c7ccccc7)ccc6c45)cc23)cc1]

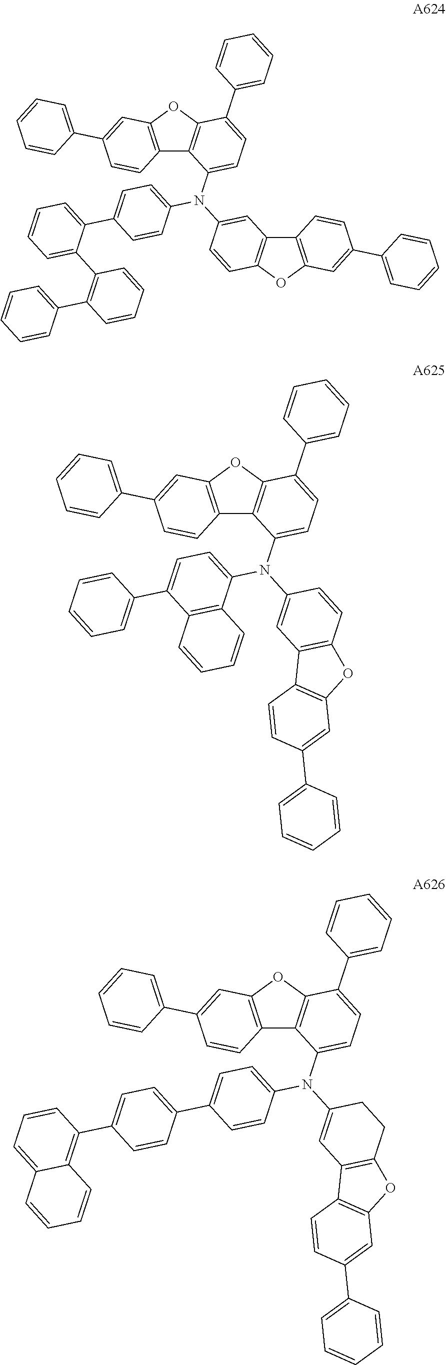 [SMILES: c1ccc(-c2ccc3c(c2)oc2ccc(N(c4ccc(-c5ccccc5-c5ccccc5-c5ccccc5)cc4)c4ccc(-c5ccccc5)c5oc6cc(-c7ccccc7)ccc6c45)cc23)cc1 ; c1ccc(-c2ccc3c(c2)oc2ccc(N(c4ccc(-c5ccccc5)c5ccccc45)c4ccc(-c5ccccc5)c5oc6cc(-c7ccccc7)ccc6c45)cc23)cc1 ; C1=C(N(c2ccc(-c3ccc(-c4cccc5ccccc45)cc3)cc2)c2ccc(-c3ccccc3)c3oc4cc(-c5ccccc5)ccc4c23)CCc2oc3cc(-c4ccccc4)ccc3c21]

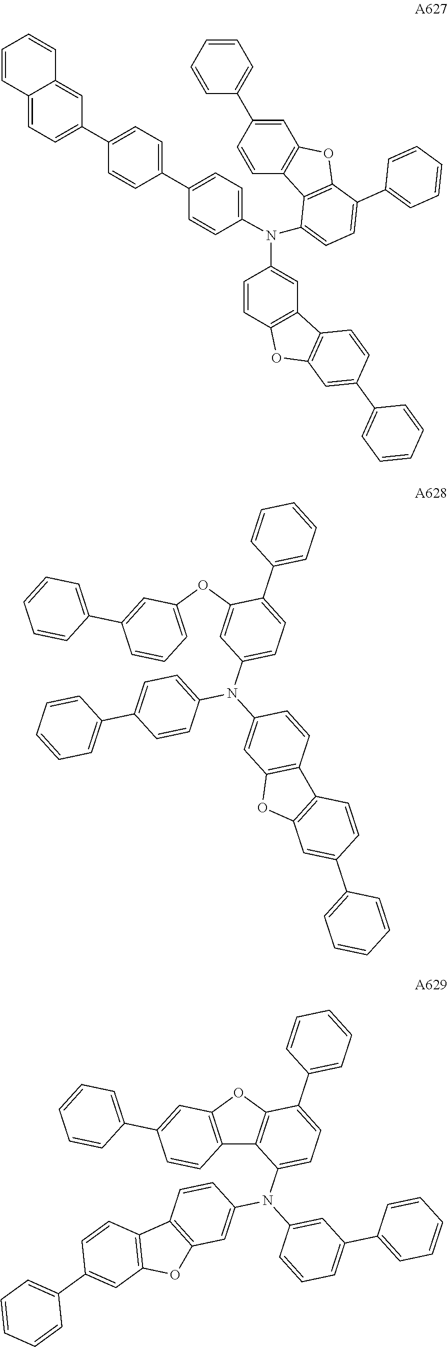 [SMILES: c1ccc(-c2ccc3c(c2)oc2ccc(N(c4ccc(-c5ccc(-c6ccc7ccccc7c6)cc5)cc4)c4ccc(-c5ccccc5)c5oc6cc(-c7ccccc7)ccc6c45)cc23)cc1 ; c1ccc(-c2ccc(N(c3ccc(-c4ccccc4)c(Oc4cccc(-c5ccccc5)c4)c3)c3ccc4c(c3)oc3cc(-c5ccccc5)ccc34)cc2)cc1 ; c1ccc(-c2cccc(N(c3ccc4c(c3)oc3cc(-c5ccccc5)ccc34)c3ccc(-c4ccccc4)c4oc5cc(-c6ccccc6)ccc5c34)c2)cc1]

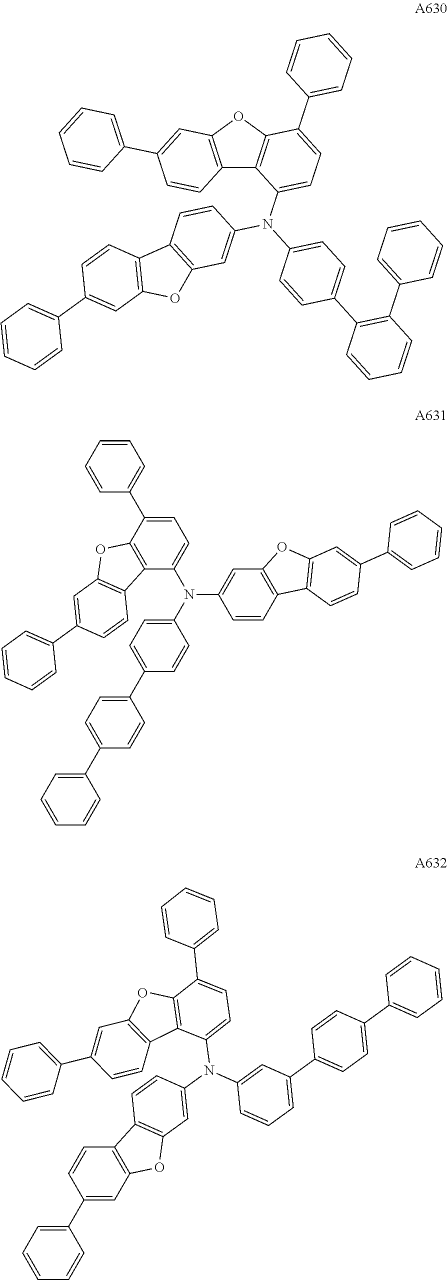 [SMILES: c1ccc(-c2ccc3c(c2)oc2cc(N(c4ccc(-c5ccccc5-c5ccccc5)cc4)c4ccc(-c5ccccc5)c5oc6cc(-c7ccccc7)ccc6c45)ccc23)cc1 ; c1ccc(-c2ccc(-c3ccc(N(c4ccc5c(c4)oc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)oc4cc(-c6ccccc6)ccc45)c4ccc(-c5ccccc5)c5oc6cc(-c7ccccc7)ccc6c45)c3)cc2)cc1]

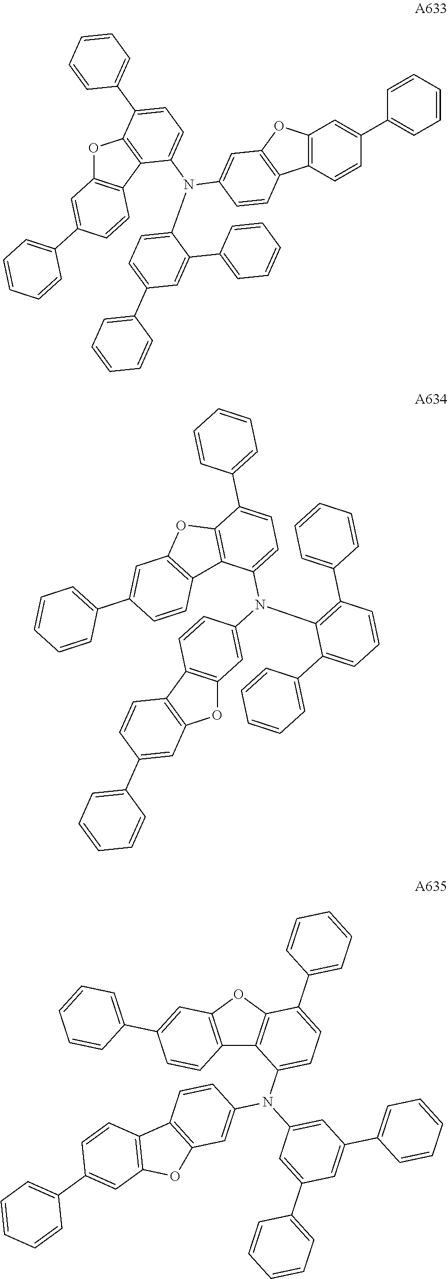 [SMILES: c1ccc(-c2ccc(N(c3ccc4c(c3)oc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2cc(N(c4c(-c5ccccc5)cccc4-c4ccccc4)c4ccc(-c5ccccc5)c5oc6cc(-c7ccccc7)ccc6c45)ccc23)cc1 ; c1ccc(-c2cc(-c3ccccc3)cc(N(c3ccc4c(c3)oc3cc(-c5ccccc5)ccc34)c3ccc(-c4ccccc4)c4oc5cc(-c6ccccc6)ccc5c34)c2)cc1]

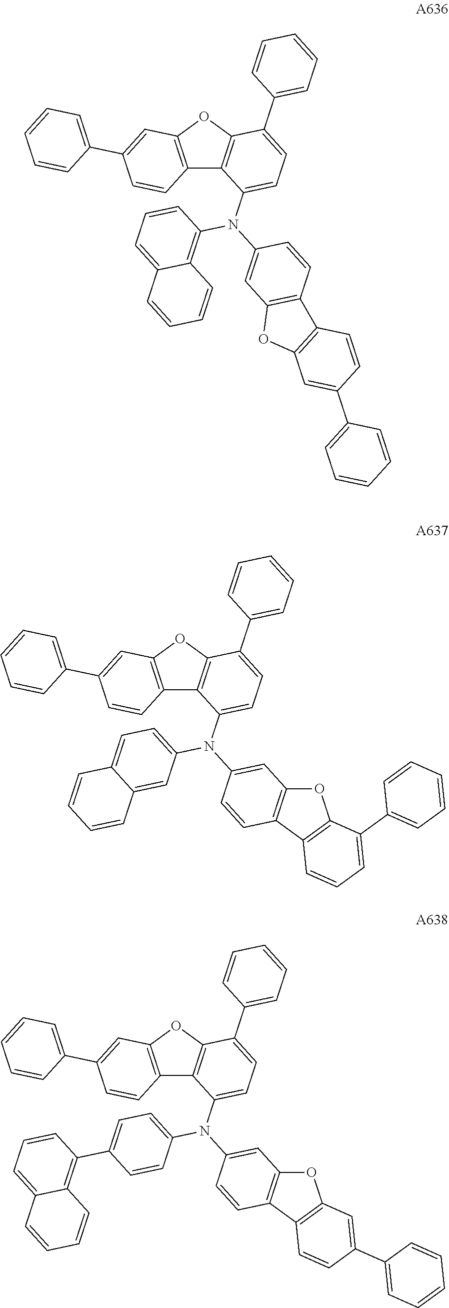 [SMILES: c1ccc(-c2ccc3c(c2)oc2cc(N(c4cccc5ccccc45)c4ccc(-c5ccccc5)c5oc6cc(-c7ccccc7)ccc6c45)ccc23)cc1 ; c1ccc(-c2ccc3c(c2)oc2c(-c4ccccc4)ccc(N(c4ccc5ccccc5c4)c4ccc5c(c4)oc4c(-c6ccccc6)cccc45)c23)cc1 ; c1ccc(-c2ccc3c(c2)oc2cc(N(c4ccc(-c5cccc6ccccc56)cc4)c4ccc(-c5ccccc5)c5oc6cc(-c7ccccc7)ccc6c45)ccc23)cc1]

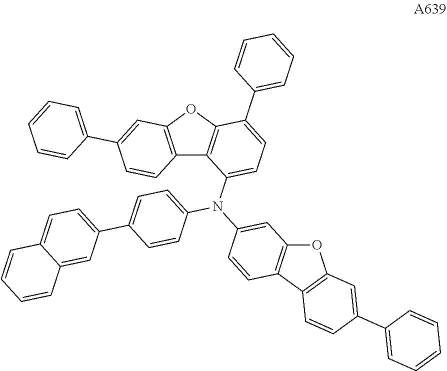 [SMILES: c1ccc(-c2ccc3c(c2)oc2cc(N(c4ccc(-c5ccc6ccccc6c5)cc4)c4ccc(-c5ccccc5)c5oc6cc(-c7ccccc7)ccc6c45)ccc23)cc1]

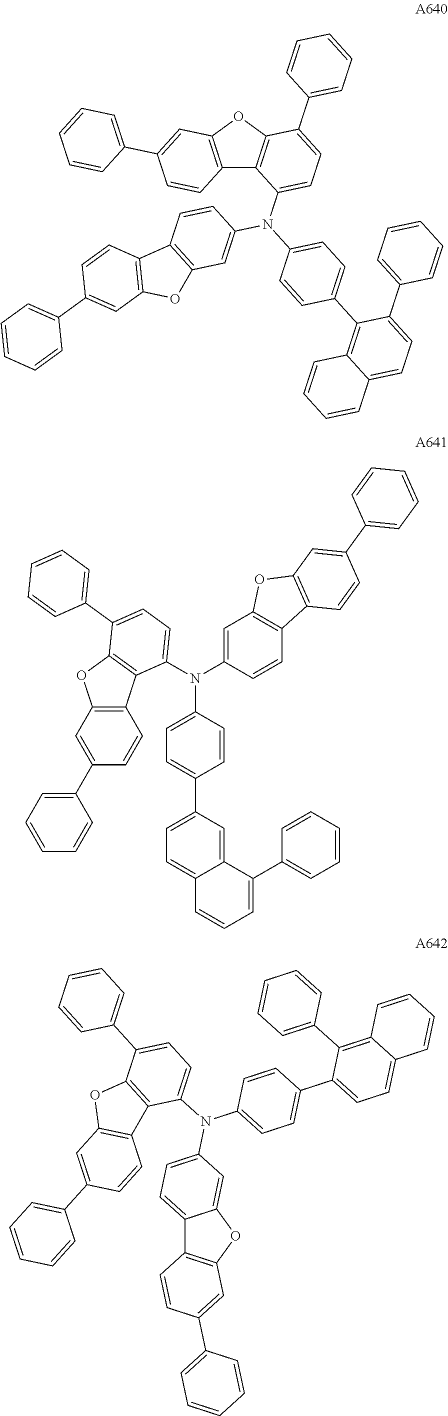 [SMILES: c1ccc(-c2ccc3c(c2)oc2cc(N(c4ccc(-c5c(-c6ccccc6)ccc6ccccc56)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6cccc(-c7ccccc7)c6c5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc6ccccc6c5-c5ccccc5)cc4)c4ccc(-c5ccccc5)c5oc6cc(-c7ccccc7)ccc6c45)ccc23)cc1]

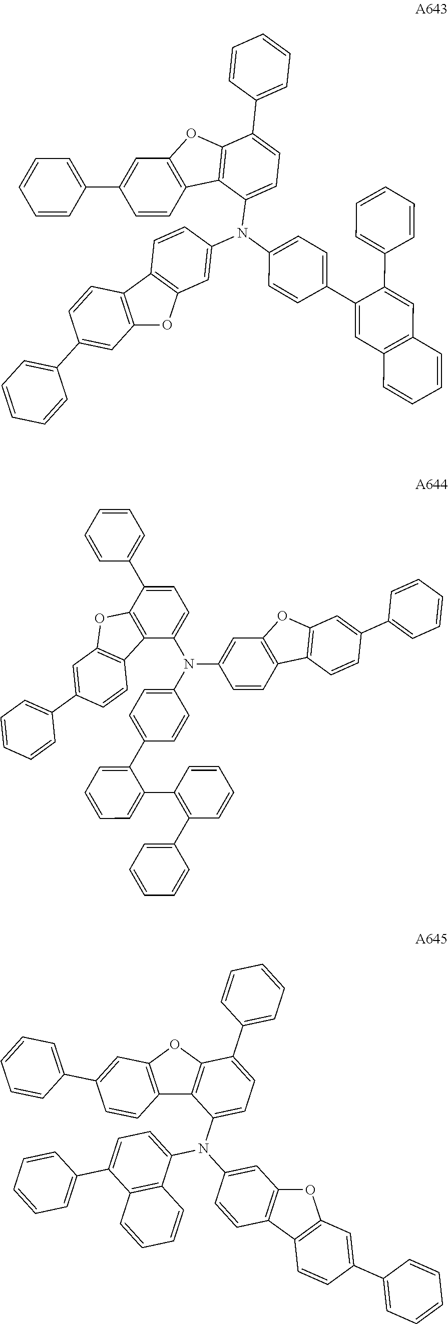 [SMILES: c1ccc(-c2ccc3c(c2)oc2cc(N(c4ccc(-c5cc6ccccc6cc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5-c5ccccc5-c5ccccc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccccc5)c5ccccc45)c4ccc(-c5ccccc5)c5oc6cc(-c7ccccc7)ccc6c45)ccc23)cc1]

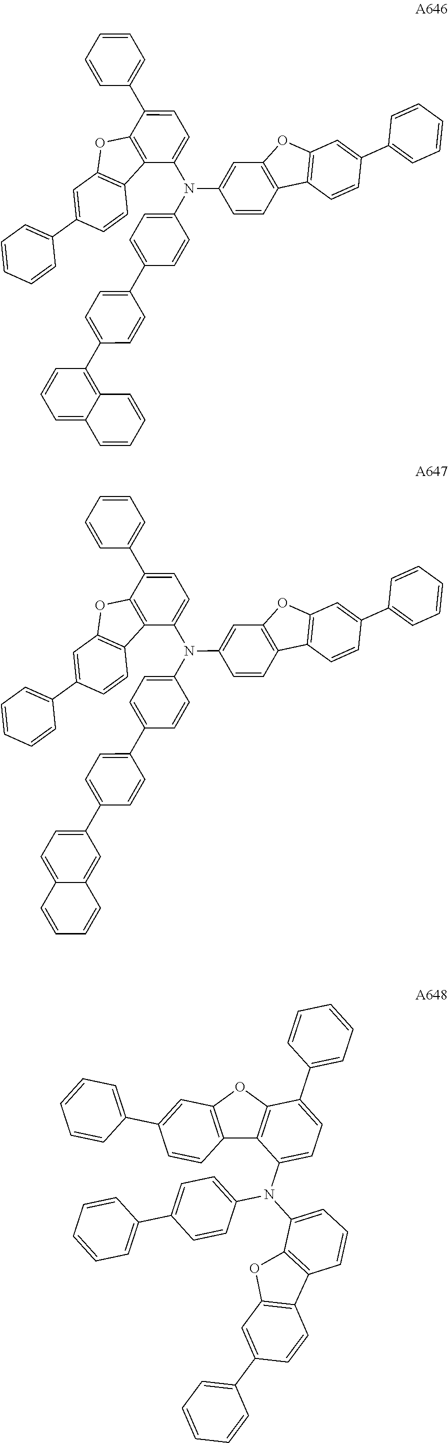 [SMILES: c1ccc(-c2ccc3c(c2)oc2cc(N(c4ccc(-c5ccc(-c6cccc7ccccc67)cc5)cc4)c4ccc(-c5ccccc5)c5oc6cc(-c7ccccc7)ccc6c45)ccc23)cc1 ; c1ccc(-c2ccc3c(c2)oc2cc(N(c4ccc(-c5ccc(-c6ccc7ccccc7c6)cc5)cc4)c4ccc(-c5ccccc5)c5oc6cc(-c7ccccc7)ccc6c45)ccc23)cc1 ; c1ccc(-c2ccc(N(c3cccc4c3oc3cc(-c5ccccc5)ccc34)c3ccc(-c4ccccc4)c4oc5cc(-c6ccccc6)ccc5c34)cc2)cc1]

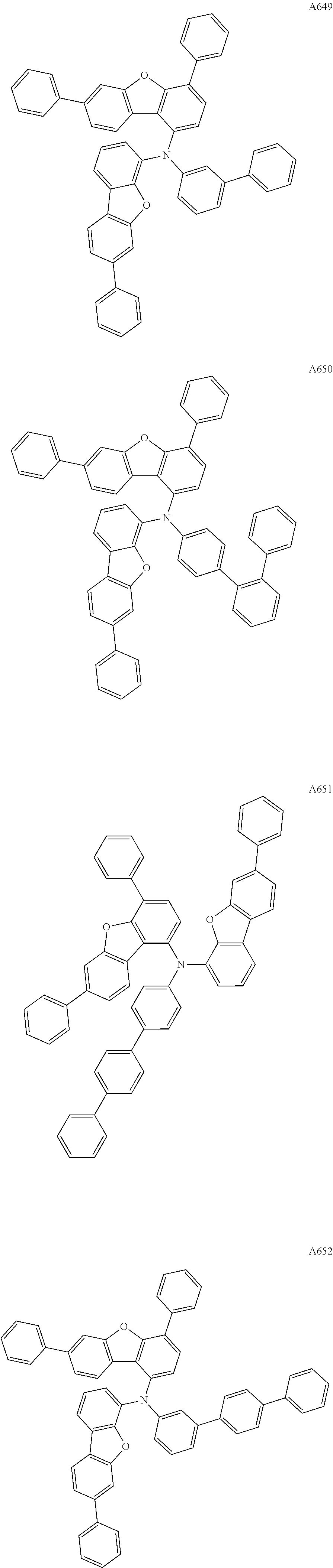 [SMILES: c1ccc(-c2cccc(N(c3cccc4c3oc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(N(c4ccc(-c5ccccc5-c5ccccc5)cc4)c4ccc(-c5ccccc5)c5oc6cc(-c7ccccc7)ccc6c45)cccc23)cc1 ; c1ccc(-c2ccc(-c3ccc(N(c4cccc5c4oc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4cccc5c4oc4cc(-c6ccccc6)ccc45)c4ccc(-c5ccccc5)c5oc6cc(-c7ccccc7)ccc6c45)c3)cc2)cc1]

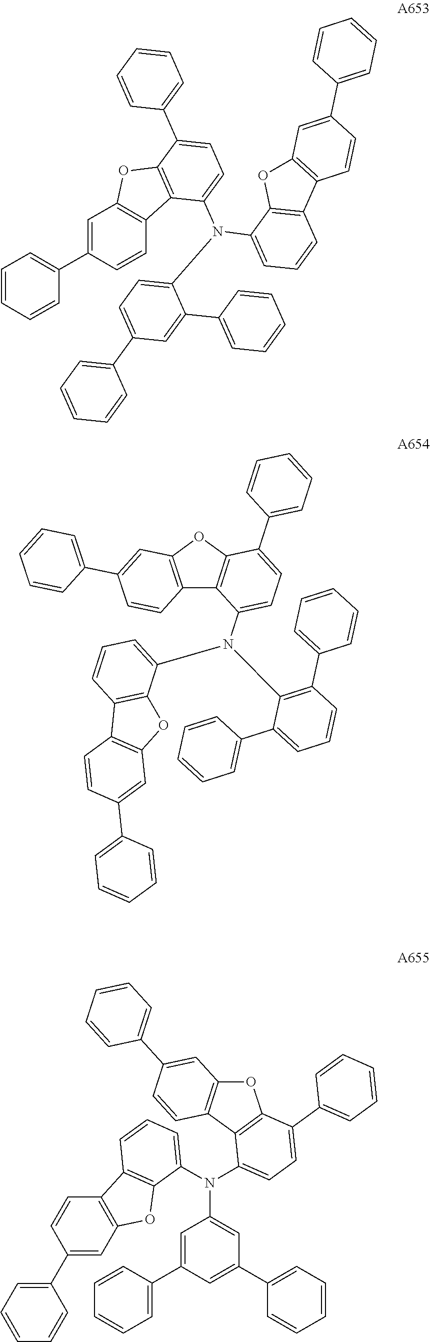 [SMILES: c1ccc(-c2ccc(N(c3cccc4c3oc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(N(c4c(-c5ccccc5)cccc4-c4ccccc4)c4ccc(-c5ccccc5)c5oc6cc(-c7ccccc7)ccc6c45)cccc23)cc1 ; c1ccc(-c2cc(-c3ccccc3)cc(N(c3cccc4c3oc3cc(-c5ccccc5)ccc34)c3ccc(-c4ccccc4)c4oc5cc(-c6ccccc6)ccc5c34)c2)cc1]

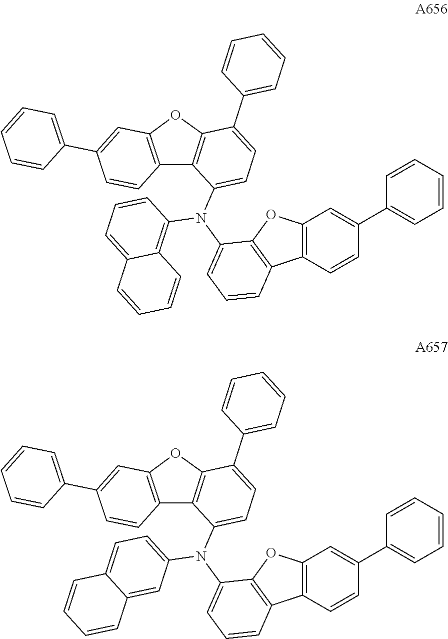 [SMILES: c1ccc(-c2ccc3c(c2)oc2c(N(c4cccc5ccccc45)c4ccc(-c5ccccc5)c5oc6cc(-c7ccccc7)ccc6c45)cccc23)cc1 ; c1ccc(-c2ccc3c(c2)oc2c(N(c4ccc5ccccc5c4)c4ccc(-c5ccccc5)c5oc6cc(-c7ccccc7)ccc6c45)cccc23)cc1]

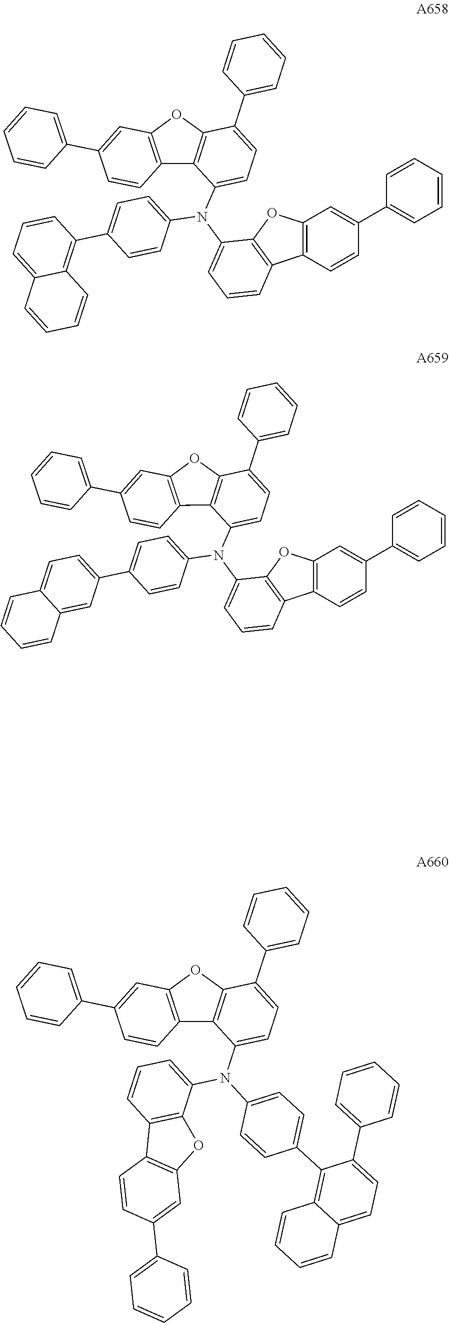 [SMILES: c1ccc(-c2ccc3c(c2)oc2c(N(c4ccc(-c5cccc6ccccc56)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6ccccc6c5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5c(-c6ccccc6)ccc6ccccc56)cc4)c4ccc(-c5ccccc5)c5oc6cc(-c7ccccc7)ccc6c45)cccc23)cc1]

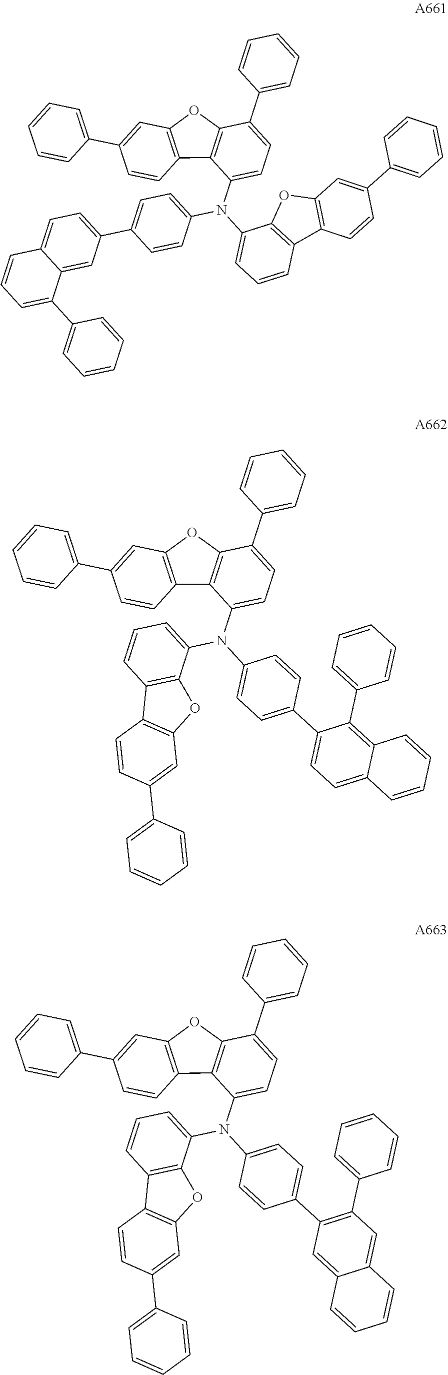 [SMILES: c1ccc(-c2ccc3c(c2)oc2c(N(c4ccc(-c5ccc6cccc(-c7ccccc7)c6c5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccc6ccccc6c5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5cc6ccccc6cc5-c5ccccc5)cc4)c4ccc(-c5ccccc5)c5oc6cc(-c7ccccc7)ccc6c45)cccc23)cc1]

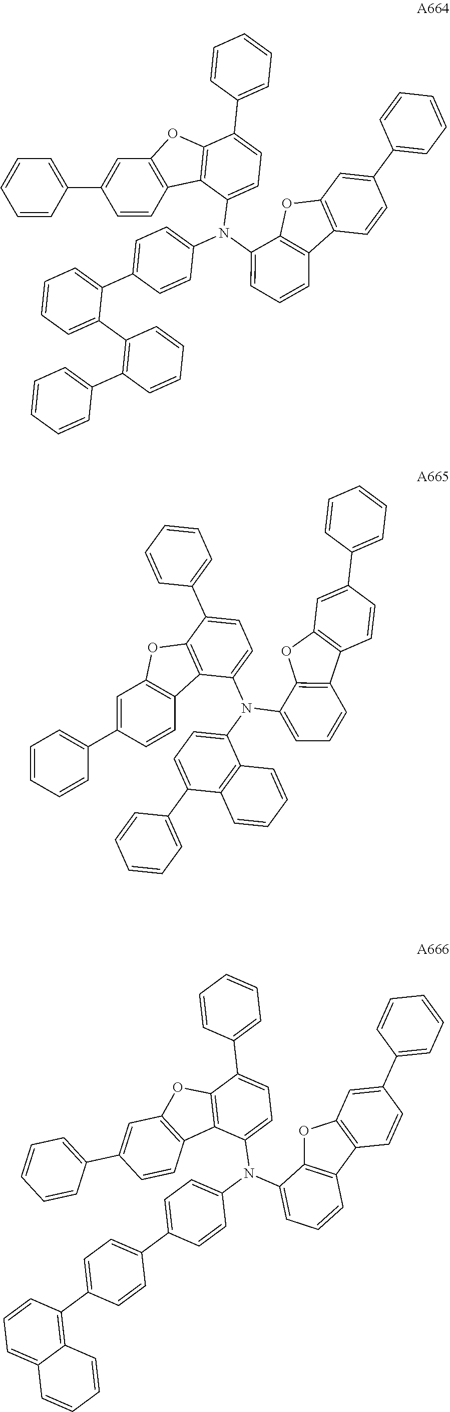 [SMILES: c1ccc(-c2ccc3c(c2)oc2c(N(c4ccc(-c5ccccc5-c5ccccc5-c5ccccc5)cc4)c4ccc(-c5ccccc5)c5oc6cc(-c7ccccc7)ccc6c45)cccc23)cc1 ; c1ccc(-c2ccc3c(c2)oc2c(N(c4ccc(-c5ccccc5)c5ccccc45)c4ccc(-c5ccccc5)c5oc6cc(-c7ccccc7)ccc6c45)cccc23)cc1 ; c1ccc(-c2ccc3c(c2)oc2c(N(c4ccc(-c5ccc(-c6cccc7ccccc67)cc5)cc4)c4ccc(-c5ccccc5)c5oc6cc(-c7ccccc7)ccc6c45)cccc23)cc1]

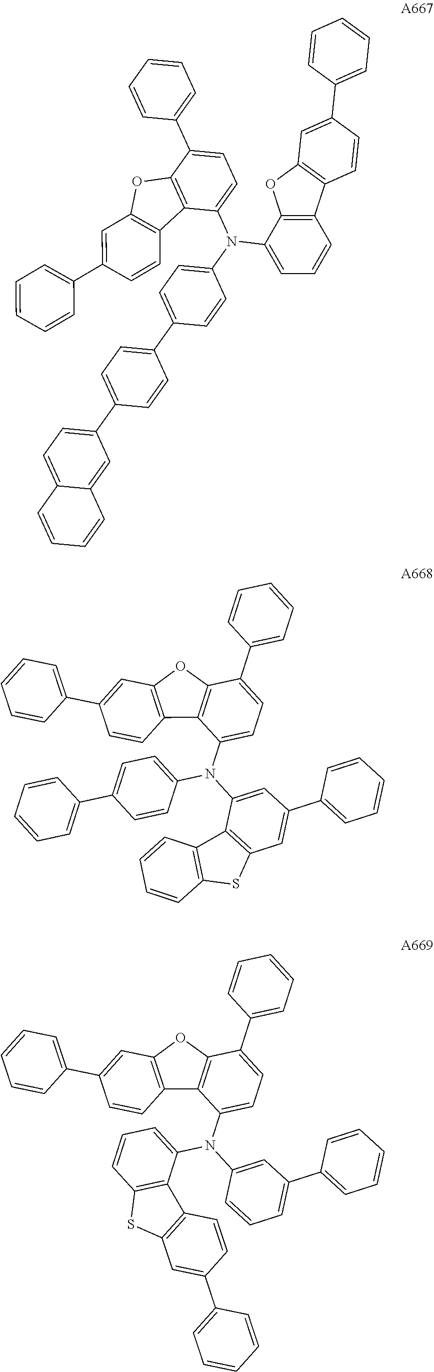 [SMILES: c1ccc(-c2ccc3c(c2)oc2c(N(c4ccc(-c5ccc(-c6ccc7ccccc7c6)cc5)cc4)c4ccc(-c5ccccc5)c5oc6cc(-c7ccccc7)ccc6c45)cccc23)cc1 ; c1ccc(-c2ccc(N(c3cc(-c4ccccc4)cc4sc5ccccc5c34)c3ccc(-c4ccccc4)c4oc5cc(-c6ccccc6)ccc5c34)cc2)cc1 ; c1ccc(-c2cccc(N(c3cccc4sc5cc(-c6ccccc6)ccc5c34)c3ccc(-c4ccccc4)c4oc5cc(-c6ccccc6)ccc5c34)c2)cc1]

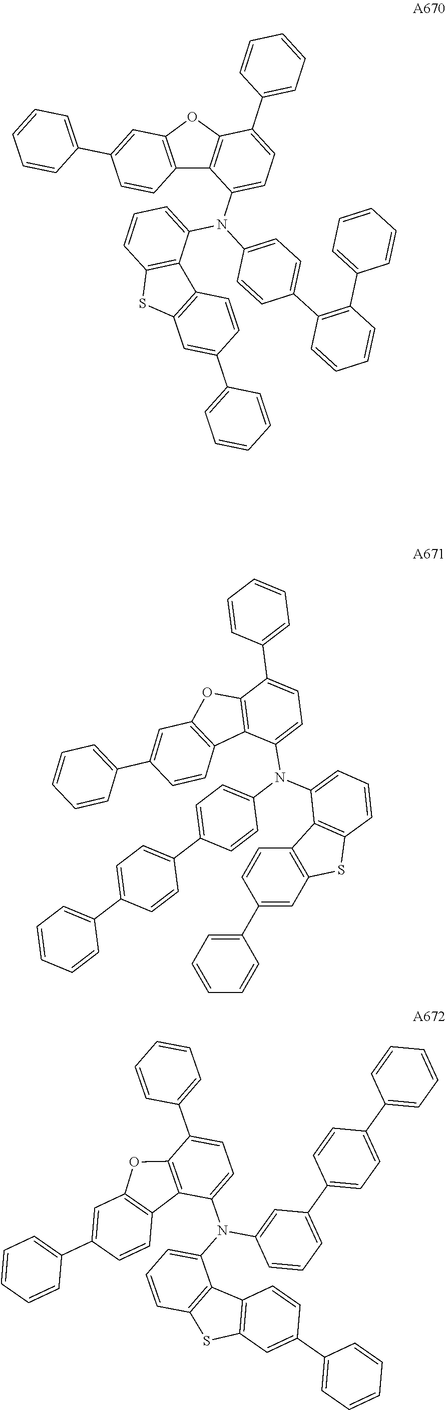 [SMILES: c1ccc(-c2ccc3c(c2)oc2c(-c4ccccc4)ccc(N(c4ccc(-c5ccccc5-c5ccccc5)cc4)c4cccc5sc6cc(-c7ccccc7)ccc6c45)c23)cc1 ; c1ccc(-c2ccc(-c3ccc(N(c4cccc5sc6cc(-c7ccccc7)ccc6c45)c4ccc(-c5ccccc5)c5oc6cc(-c7ccccc7)ccc6c45)cc3)cc2)cc1 ; c1ccc(-c2ccc(-c3cccc(N(c4cccc5sc6cc(-c7ccccc7)ccc6c45)c4ccc(-c5ccccc5)c5oc6cc(-c7ccccc7)ccc6c45)c3)cc2)cc1]

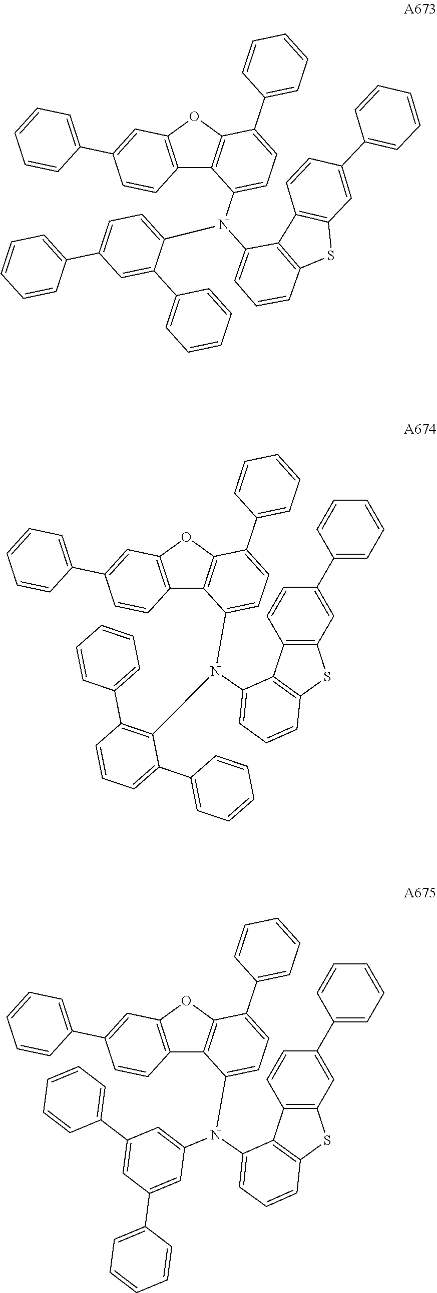 [SMILES: c1ccc(-c2ccc(N(c3cccc4sc5cc(-c6ccccc6)ccc5c34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5sc6cc(-c7ccccc7)ccc6c45)c23)cc1 ; c1ccc(-c2cc(-c3ccccc3)cc(N(c3cccc4sc5cc(-c6ccccc6)ccc5c34)c3ccc(-c4ccccc4)c4oc5cc(-c6ccccc6)ccc5c34)c2)cc1]

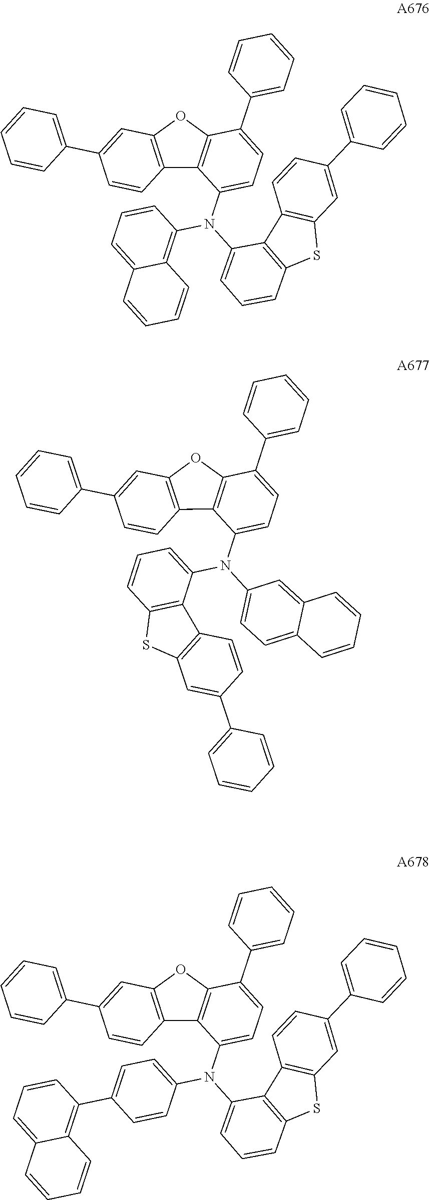 [SMILES: c1ccc(-c2ccc3c(c2)oc2c(-c4ccccc4)ccc(N(c4cccc5ccccc45)c4cccc5sc6cc(-c7ccccc7)ccc6c45)c23)cc1 ; c1ccc(-c2ccc3c(c2)oc2c(-c4ccccc4)ccc(N(c4ccc5ccccc5c4)c4cccc5sc6cc(-c7ccccc7)ccc6c45)c23)cc1 ; c1ccc(-c2ccc3c(c2)oc2c(-c4ccccc4)ccc(N(c4ccc(-c5cccc6ccccc56)cc4)c4cccc5sc6cc(-c7ccccc7)ccc6c45)c23)cc1]

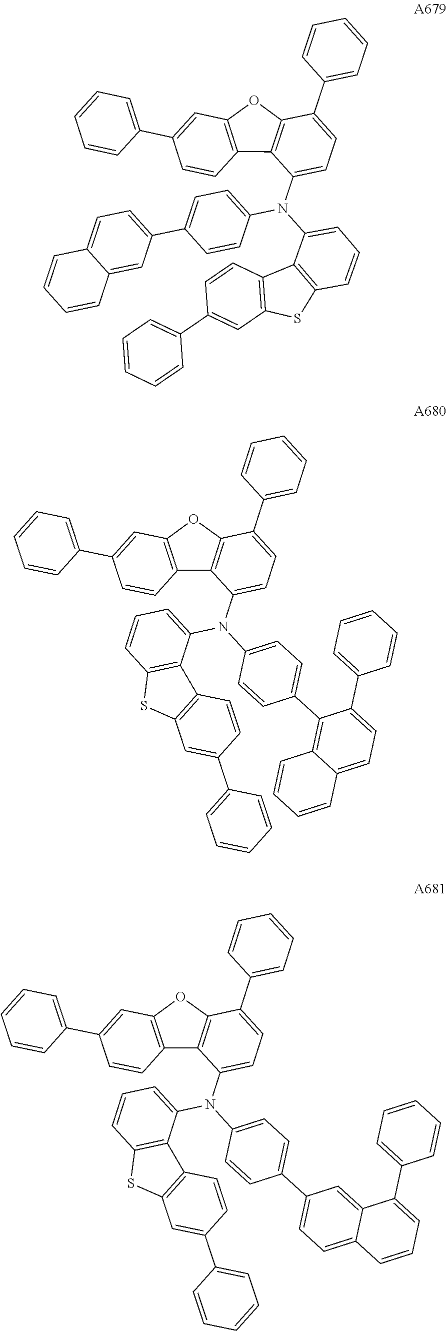 [SMILES: c1ccc(-c2ccc3c(c2)oc2c(-c4ccccc4)ccc(N(c4ccc(-c5ccc6ccccc6c5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5c(-c6ccccc6)ccc6ccccc56)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc6cccc(-c7ccccc7)c6c5)cc4)c4cccc5sc6cc(-c7ccccc7)ccc6c45)c23)cc1]

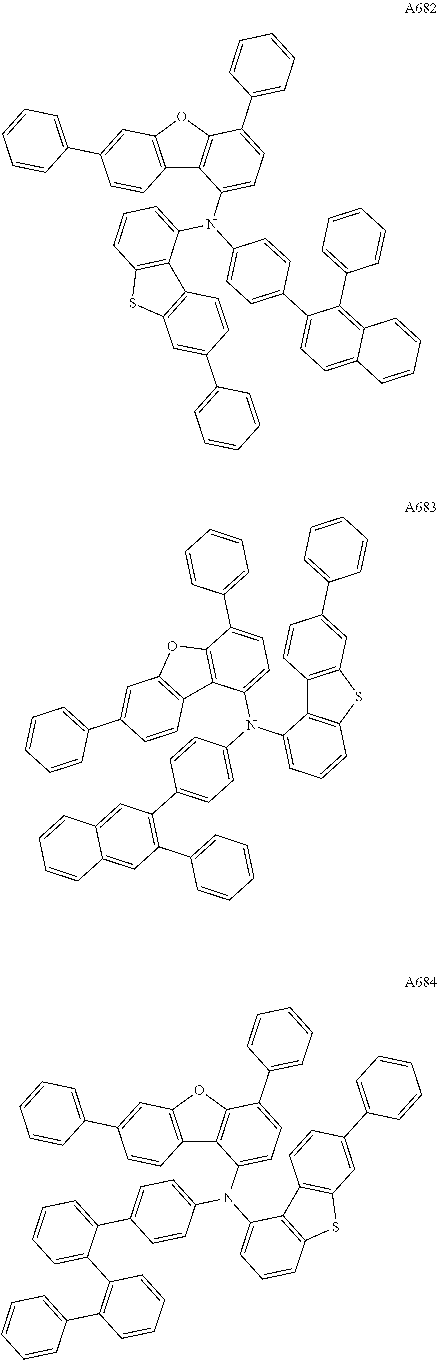 [SMILES: c1ccc(-c2ccc3c(c2)oc2c(-c4ccccc4)ccc(N(c4ccc(-c5ccc6ccccc6c5-c5ccccc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5cc6ccccc6cc5-c5ccccc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccccc5-c5ccccc5-c5ccccc5)cc4)c4cccc5sc6cc(-c7ccccc7)ccc6c45)c23)cc1]

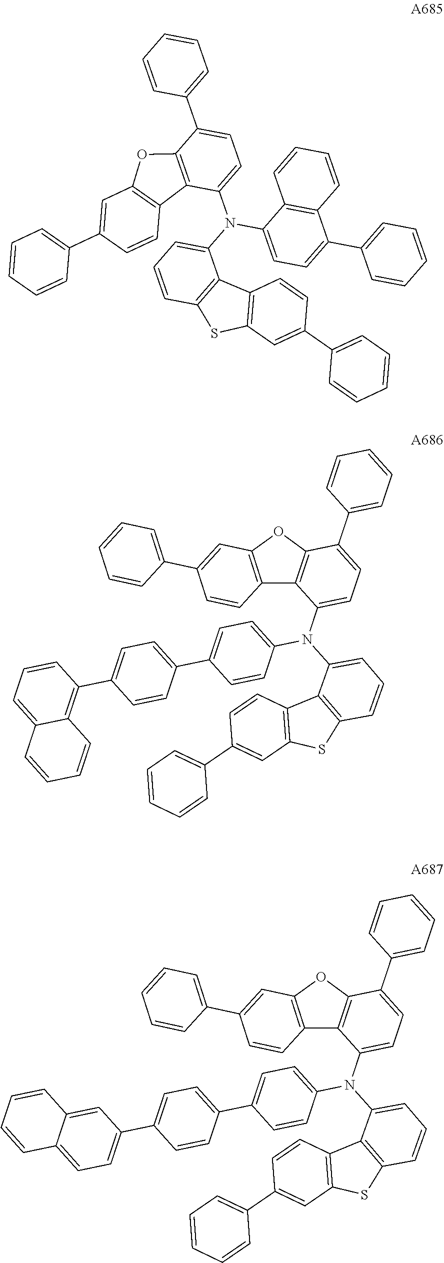 [SMILES: c1ccc(-c2ccc3c(c2)oc2c(-c4ccccc4)ccc(N(c4ccc(-c5ccccc5)c5ccccc45)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6cccc7ccccc67)cc5)cc4)c4cccc5sc6cc(-c7ccccc7)ccc6c45)c23)cc1 ; c1ccc(-c2ccc3c(c2)oc2c(-c4ccccc4)ccc(N(c4ccc(-c5ccc(-c6ccc7ccccc7c6)cc5)cc4)c4cccc5sc6cc(-c7ccccc7)ccc6c45)c23)cc1]

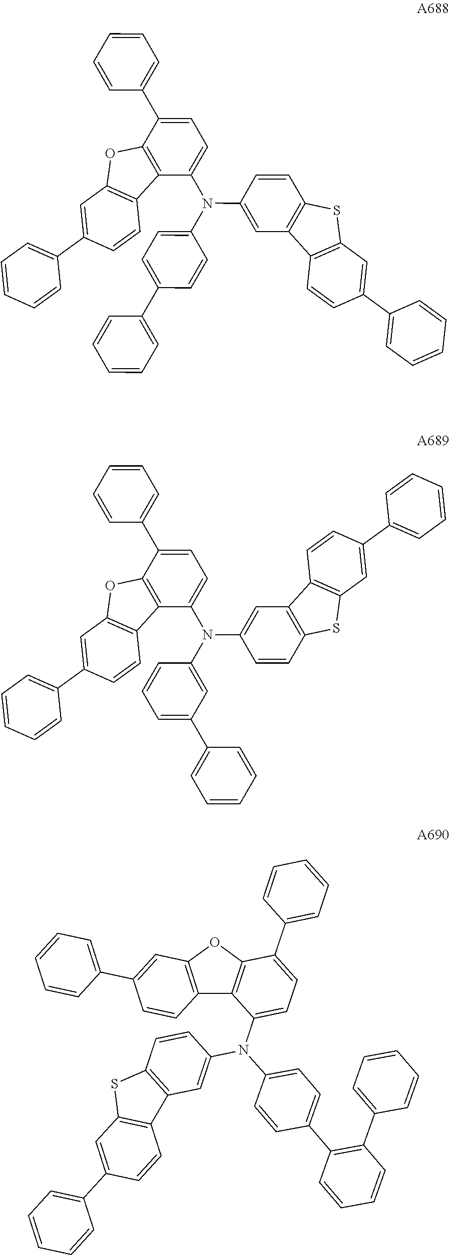 [SMILES: c1ccc(-c2ccc(N(c3ccc4sc5cc(-c6ccccc6)ccc5c4c3)c3ccc(-c4ccccc4)c4oc5cc(-c6ccccc6)ccc5c34)cc2)cc1 ; c1ccc(-c2cccc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc(-c5ccccc5-c5ccccc5)cc4)c4ccc5sc6cc(-c7ccccc7)ccc6c5c4)c23)cc1]

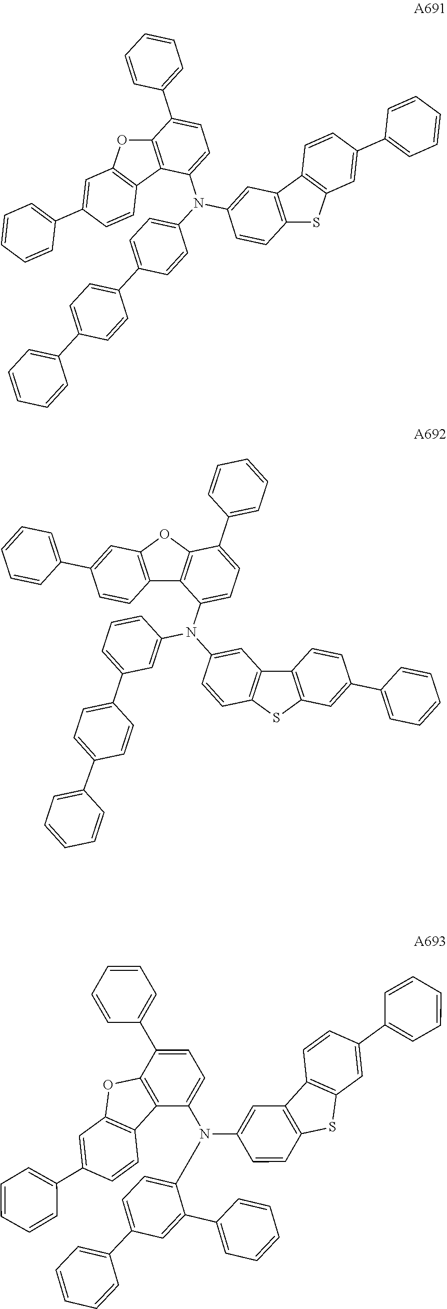 [SMILES: c1ccc(-c2ccc(-c3ccc(N(c4ccc5sc6cc(-c7ccccc7)ccc6c5c4)c4ccc(-c5ccccc5)c5oc6cc(-c7ccccc7)ccc6c45)cc3)cc2)cc1 ; c1ccc(-c2ccc(-c3cccc(N(c4ccc5sc6cc(-c7ccccc7)ccc6c5c4)c4ccc(-c5ccccc5)c5oc6cc(-c7ccccc7)ccc6c45)c3)cc2)cc1 ; c1ccc(-c2ccc(N(c3ccc4sc5cc(-c6ccccc6)ccc5c4c3)c3ccc(-c4ccccc4)c4oc5cc(-c6ccccc6)ccc5c34)c(-c3ccccc3)c2)cc1]

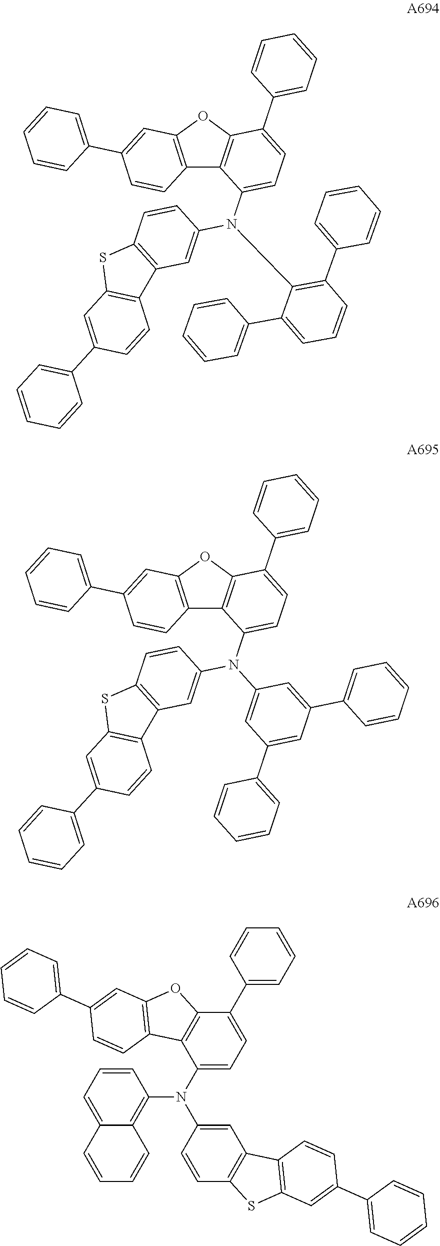 [SMILES: c1ccc(-c2ccc3c(c2)oc2c(-c4ccccc4)ccc(N(c4ccc5sc6cc(-c7ccccc7)ccc6c5c4)c4c(-c5ccccc5)cccc4-c4ccccc4)c23)cc1 ; c1ccc(-c2cc(-c3ccccc3)cc(N(c3ccc4sc5cc(-c6ccccc6)ccc5c4c3)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5sc6cc(-c7ccccc7)ccc6c5c4)c4cccc5ccccc45)c23)cc1]

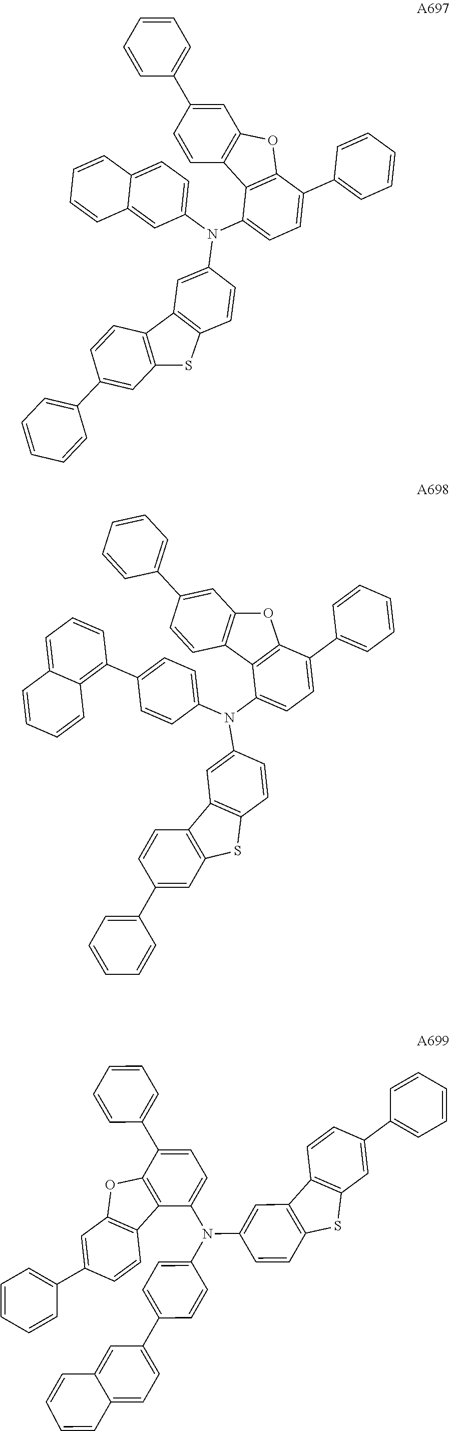 [SMILES: c1ccc(-c2ccc3c(c2)oc2c(-c4ccccc4)ccc(N(c4ccc5ccccc5c4)c4ccc5sc6cc(-c7ccccc7)ccc6c5c4)c23)cc1 ; c1ccc(-c2ccc3c(c2)oc2c(-c4ccccc4)ccc(N(c4ccc(-c5cccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5)cc4)c4ccc5sc6cc(-c7ccccc7)ccc6c5c4)c23)cc1]

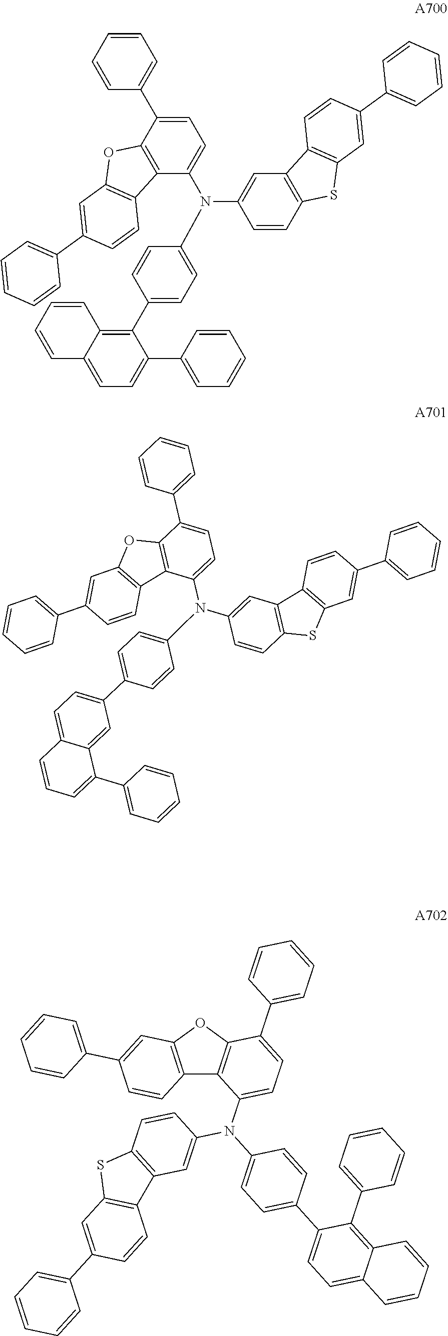 [SMILES: c1ccc(-c2ccc3c(c2)oc2c(-c4ccccc4)ccc(N(c4ccc(-c5c(-c6ccccc6)ccc6ccccc56)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6cccc(-c7ccccc7)c6c5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc6ccccc6c5-c5ccccc5)cc4)c4ccc5sc6cc(-c7ccccc7)ccc6c5c4)c23)cc1]

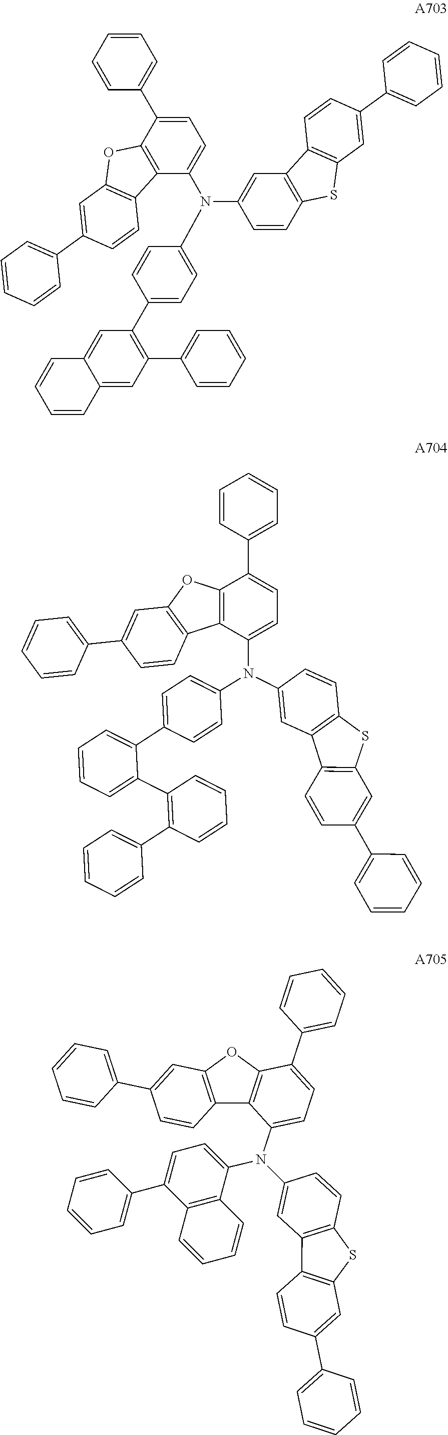 [SMILES: c1ccc(-c2ccc3c(c2)oc2c(-c4ccccc4)ccc(N(c4ccc(-c5cc6ccccc6cc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccccc5-c5ccccc5-c5ccccc5)cc4)c4ccc5sc6cc(-c7ccccc7)ccc6c5c4)c23)cc1 ; c1ccc(-c2ccc3c(c2)oc2c(-c4ccccc4)ccc(N(c4ccc5sc6cc(-c7ccccc7)ccc6c5c4)c4ccc(-c5ccccc5)c5ccccc45)c23)cc1]

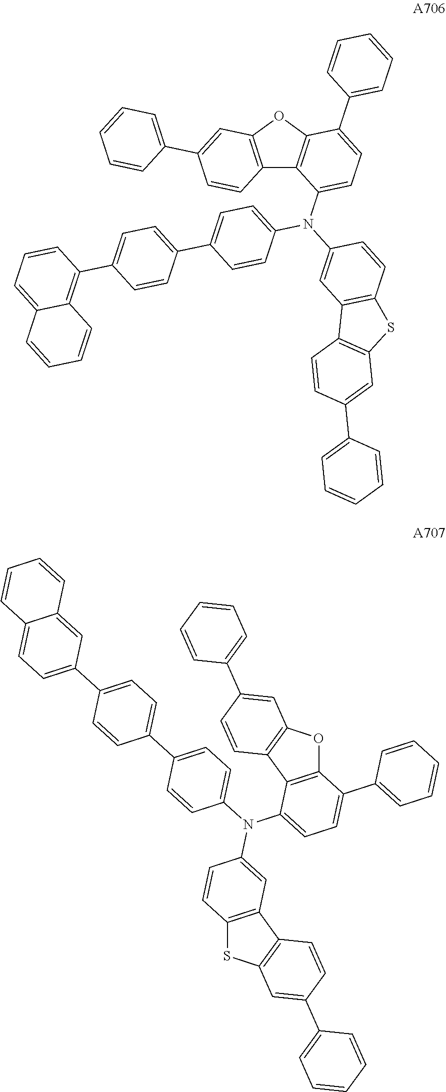 [SMILES: c1ccc(-c2ccc3c(c2)oc2c(-c4ccccc4)ccc(N(c4ccc(-c5ccc(-c6cccc7ccccc67)cc5)cc4)c4ccc5sc6cc(-c7ccccc7)ccc6c5c4)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5sc6cc(-c7ccccc7)ccc6c5c4)c23)cc1]

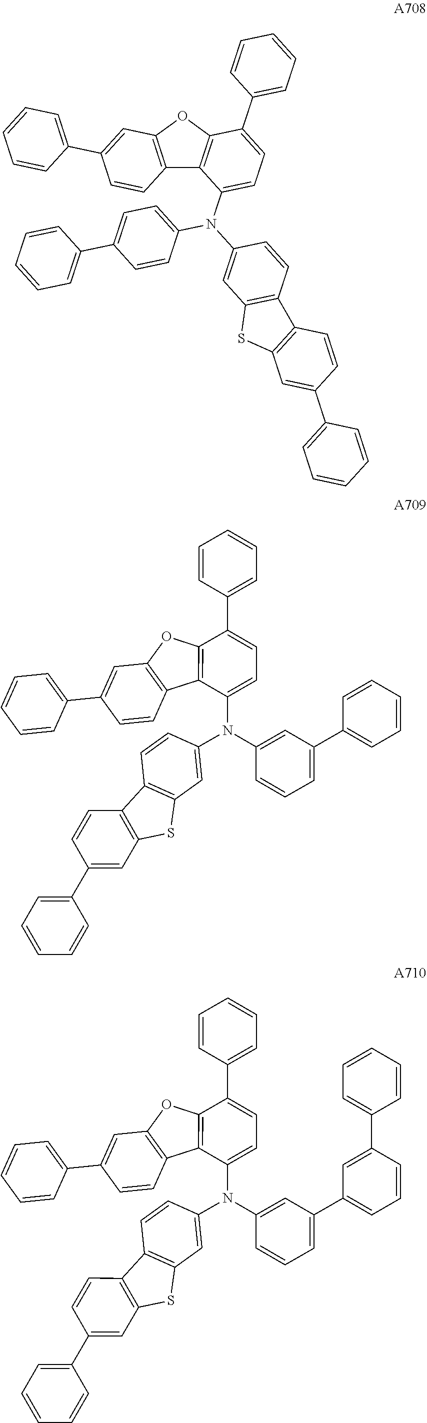 [SMILES: c1ccc(-c2ccc(N(c3ccc4c(c3)sc3cc(-c5ccccc5)ccc34)c3ccc(-c4ccccc4)c4oc5cc(-c6ccccc6)ccc5c34)cc2)cc1 ; c1ccc(-c2cccc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2cccc(-c3cccc(N(c4ccc5c(c4)sc4cc(-c6ccccc6)ccc45)c4ccc(-c5ccccc5)c5oc6cc(-c7ccccc7)ccc6c45)c3)c2)cc1]

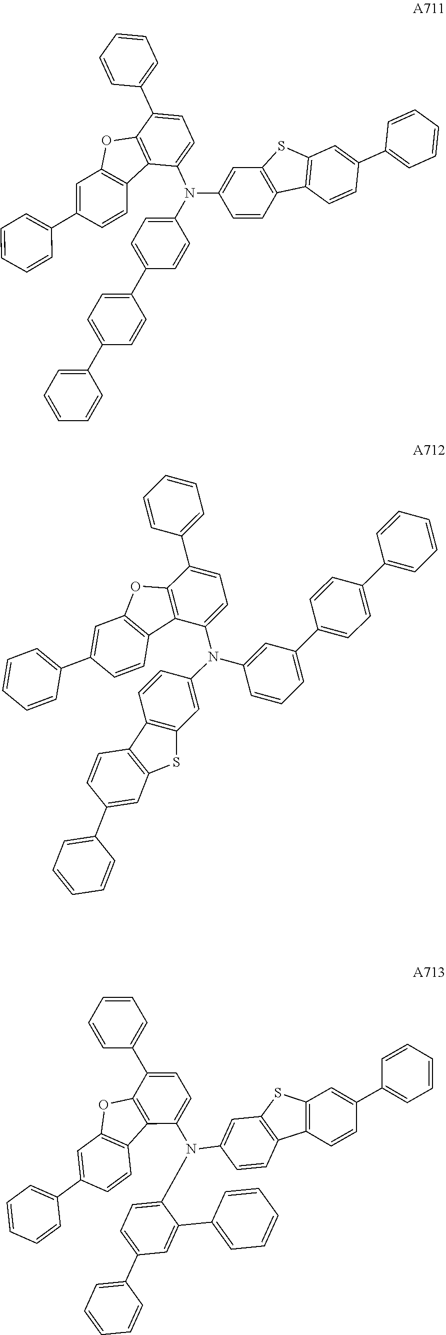 [SMILES: c1ccc(-c2ccc(-c3ccc(N(c4ccc5c(c4)sc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4ccc5c(c4)sc4cc(-c6ccccc6)ccc45)c4ccc(-c5ccccc5)c5oc6cc(-c7ccccc7)ccc6c45)c3)cc2)cc1 ; c1ccc(-c2ccc(N(c3ccc4c(c3)sc3cc(-c5ccccc5)ccc34)c3ccc(-c4ccccc4)c4oc5cc(-c6ccccc6)ccc5c34)c(-c3ccccc3)c2)cc1]

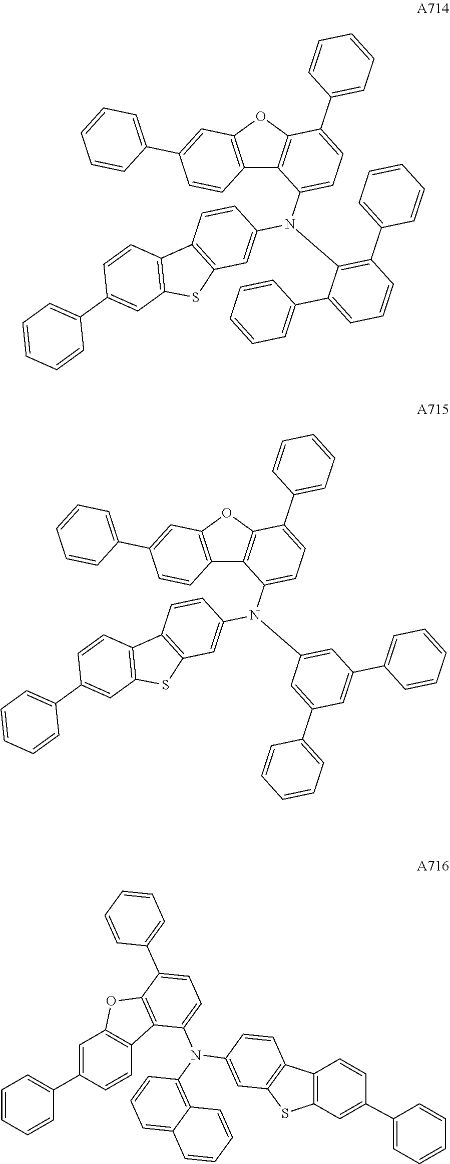 [SMILES: c1ccc(-c2ccc3c(c2)oc2c(-c4ccccc4)ccc(N(c4ccc5c(c4)sc4cc(-c6ccccc6)ccc45)c4c(-c5ccccc5)cccc4-c4ccccc4)c23)cc1 ; c1ccc(-c2cc(-c3ccccc3)cc(N(c3ccc4c(c3)sc3cc(-c5ccccc5)ccc34)c3ccc(-c4ccccc4)c4oc5cc(-c6ccccc6)ccc5c34)c2)cc1 ; c1ccc(-c2ccc3c(c2)oc2c(-c4ccccc4)ccc(N(c4ccc5c(c4)sc4cc(-c6ccccc6)ccc45)c4cccc5ccccc45)c23)cc1]

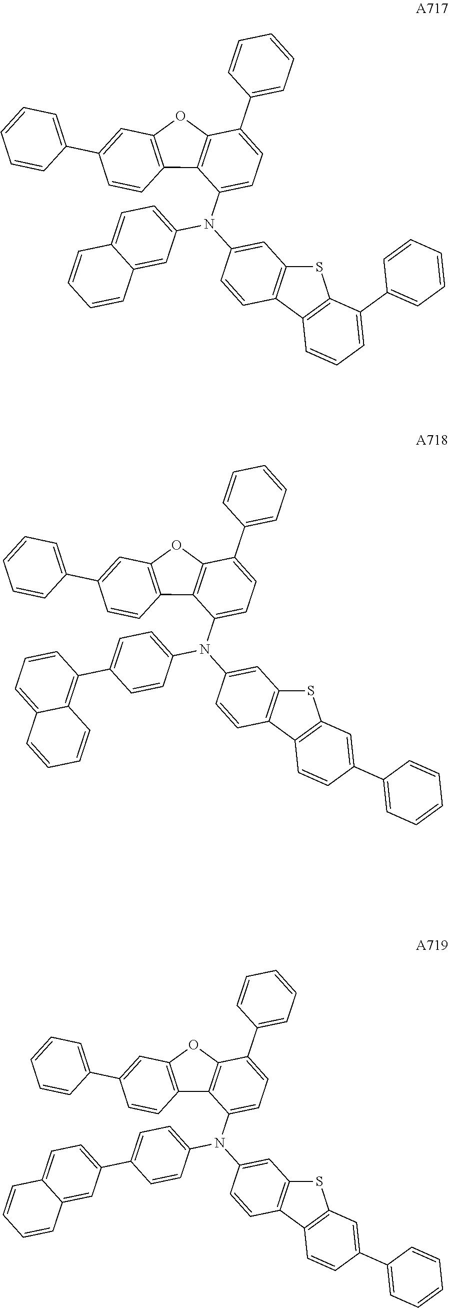 [SMILES: c1ccc(-c2ccc3c(c2)oc2c(-c4ccccc4)ccc(N(c4ccc5ccccc5c4)c4ccc5c(c4)sc4c(-c6ccccc6)cccc45)c23)cc1 ; c1ccc(-c2ccc3c(c2)oc2c(-c4ccccc4)ccc(N(c4ccc(-c5cccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5)cc4)c4ccc5c(c4)sc4cc(-c6ccccc6)ccc45)c23)cc1]

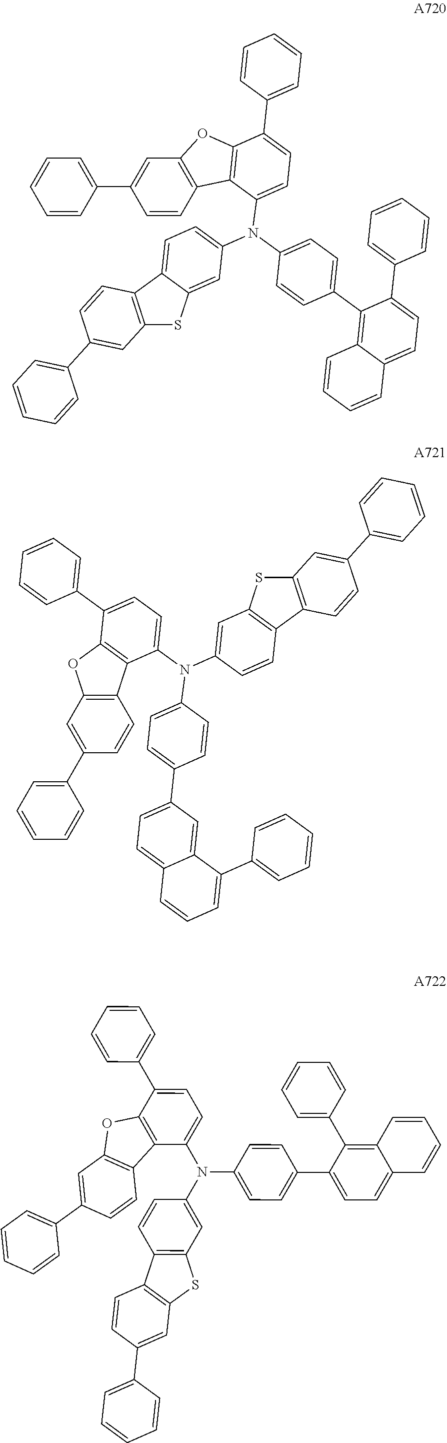 [SMILES: c1ccc(-c2ccc3c(c2)oc2c(-c4ccccc4)ccc(N(c4ccc(-c5c(-c6ccccc6)ccc6ccccc56)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6cccc(-c7ccccc7)c6c5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc6ccccc6c5-c5ccccc5)cc4)c4ccc5c(c4)sc4cc(-c6ccccc6)ccc45)c23)cc1]

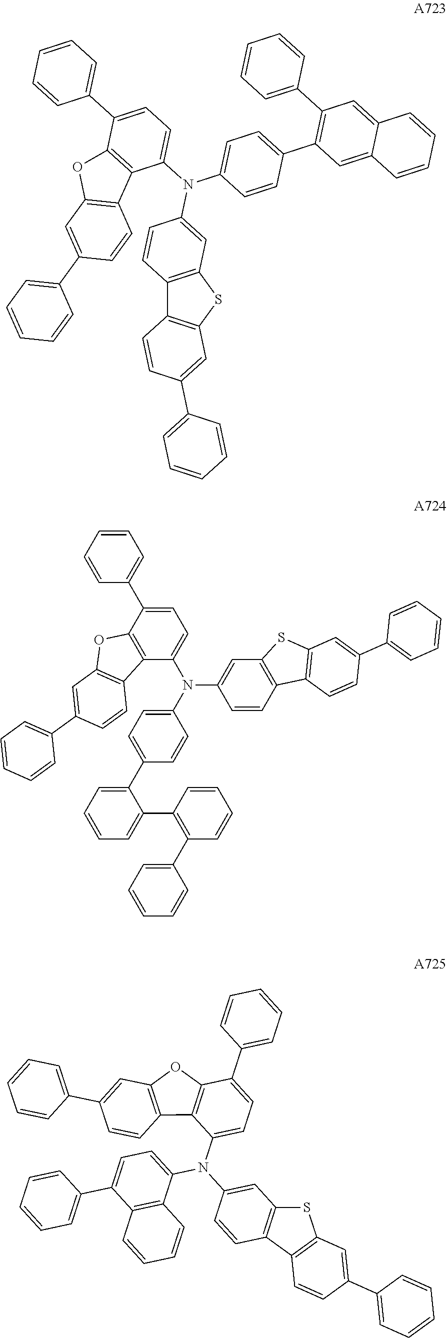 [SMILES: c1ccc(-c2ccc3c(c2)oc2c(-c4ccccc4)ccc(N(c4ccc(-c5cc6ccccc6cc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccccc5-c5ccccc5-c5ccccc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc5c(c4)sc4cc(-c6ccccc6)ccc45)c4ccc(-c5ccccc5)c5ccccc45)c23)cc1]

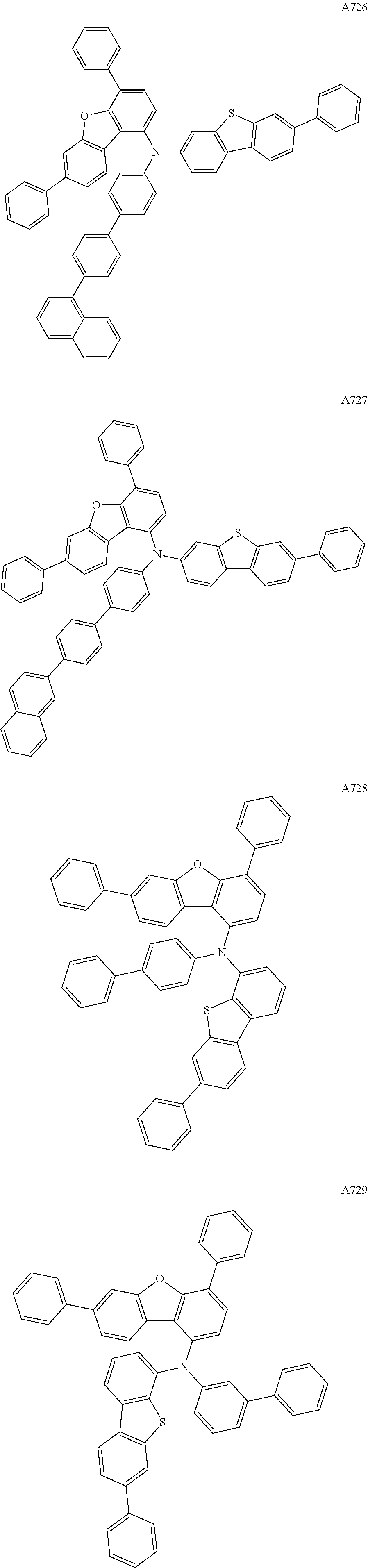 [SMILES: c1ccc(-c2ccc3c(c2)oc2c(-c4ccccc4)ccc(N(c4ccc(-c5ccc(-c6cccc7ccccc67)cc5)cc4)c4ccc5c(c4)sc4cc(-c6ccccc6)ccc45)c23)cc1 ; c1ccc(-c2ccc3c(c2)oc2c(-c4ccccc4)ccc(N(c4ccc(-c5ccc(-c6ccc7ccccc7c6)cc5)cc4)c4ccc5c(c4)sc4cc(-c6ccccc6)ccc45)c23)cc1 ; c1ccc(-c2ccc(N(c3cccc4c3sc3cc(-c5ccccc5)ccc34)c3ccc(-c4ccccc4)c4oc5cc(-c6ccccc6)ccc5c34)cc2)cc1 ; c1ccc(-c2cccc(N(c3cccc4c3sc3cc(-c5ccccc5)ccc34)c3ccc(-c4ccccc4)c4oc5cc(-c6ccccc6)ccc5c34)c2)cc1]

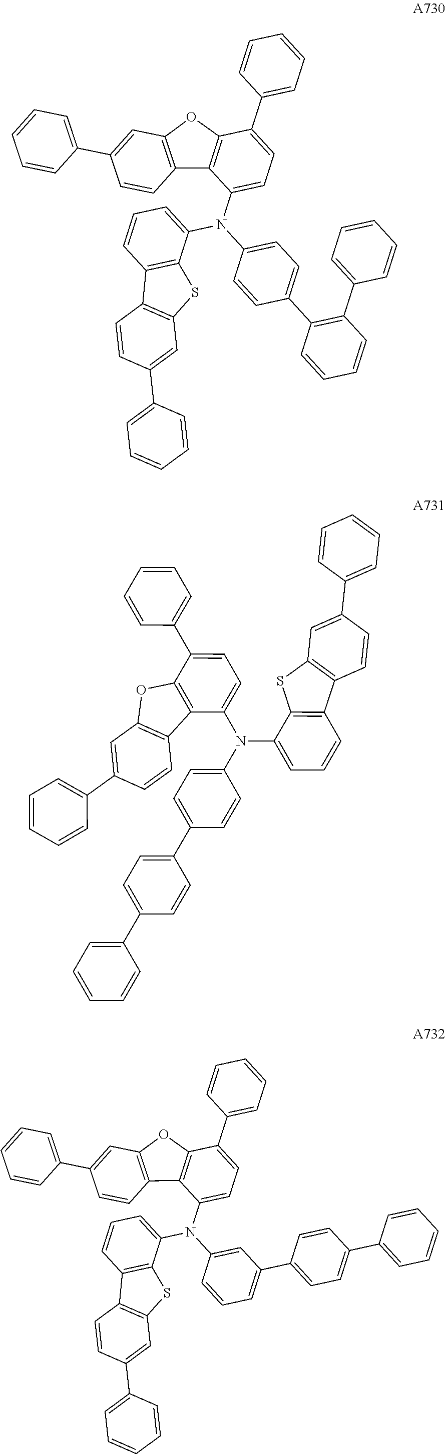 [SMILES: c1ccc(-c2ccc3c(c2)oc2c(-c4ccccc4)ccc(N(c4ccc(-c5ccccc5-c5ccccc5)cc4)c4cccc5c4sc4cc(-c6ccccc6)ccc45)c23)cc1 ; c1ccc(-c2ccc(-c3ccc(N(c4cccc5c4sc4cc(-c6ccccc6)ccc45)c4ccc(-c5ccccc5)c5oc6cc(-c7ccccc7)ccc6c45)cc3)cc2)cc1 ; c1ccc(-c2ccc(-c3cccc(N(c4cccc5c4sc4cc(-c6ccccc6)ccc45)c4ccc(-c5ccccc5)c5oc6cc(-c7ccccc7)ccc6c45)c3)cc2)cc1]

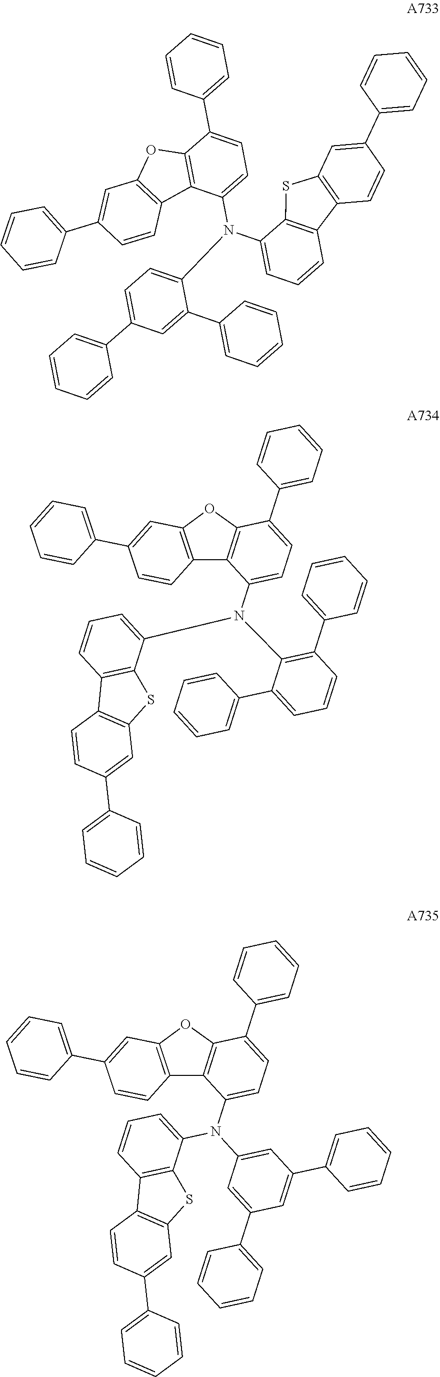 [SMILES: c1ccc(-c2ccc(N(c3cccc4c3sc3cc(-c5ccccc5)ccc34)c3ccc(-c4ccccc4)c4oc5cc(-c6ccccc6)ccc5c34)c(-c3ccccc3)c2)cc1 ; c1ccc(-c2ccc3c(c2)oc2c(-c4ccccc4)ccc(N(c4c(-c5ccccc5)cccc4-c4ccccc4)c4cccc5c4sc4cc(-c6ccccc6)ccc45)c23)cc1 ; c1ccc(-c2cc(-c3ccccc3)cc(N(c3cccc4c3sc3cc(-c5ccccc5)ccc34)c3ccc(-c4ccccc4)c4oc5cc(-c6ccccc6)ccc5c34)c2)cc1]

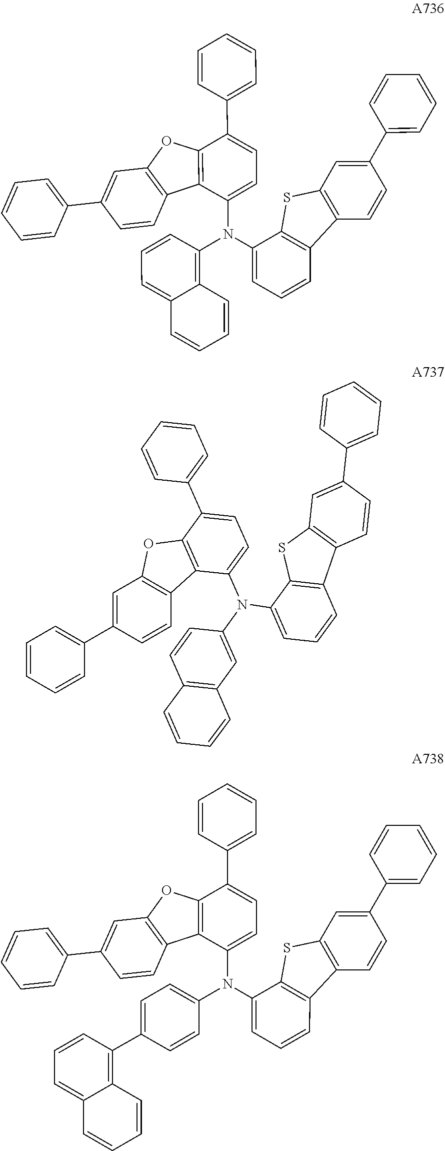

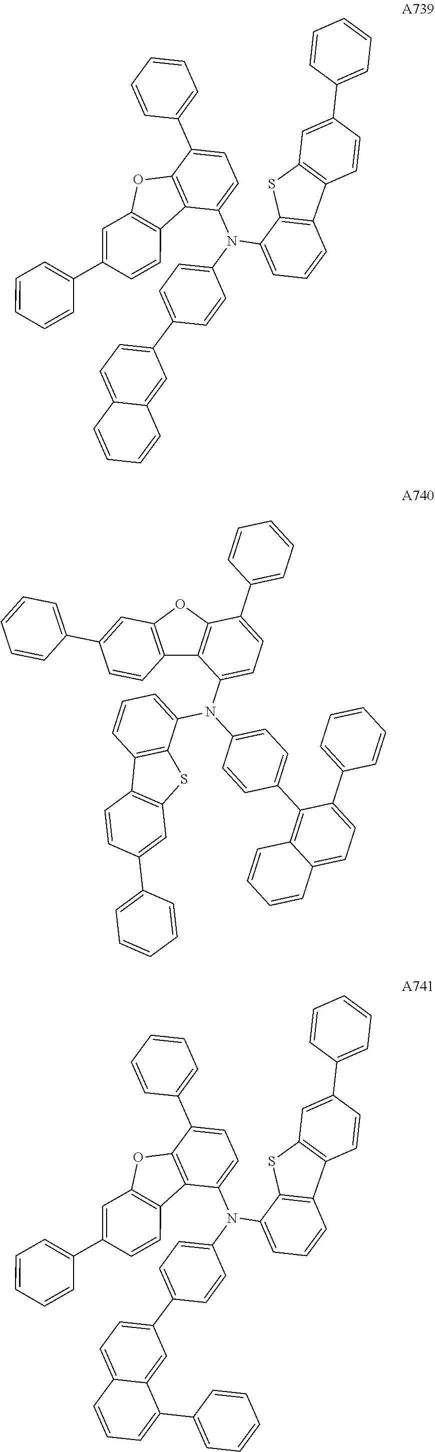 [SMILES: c1ccc(-c2ccc3c(c2)oc2c(-c4ccccc4)ccc(N(c4ccc(-c5ccc6ccccc6c5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5c(-c6ccccc6)ccc6ccccc56)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc6cccc(-c7ccccc7)c6c5)cc4)c4cccc5c4sc4cc(-c6ccccc6)ccc45)c23)cc1]

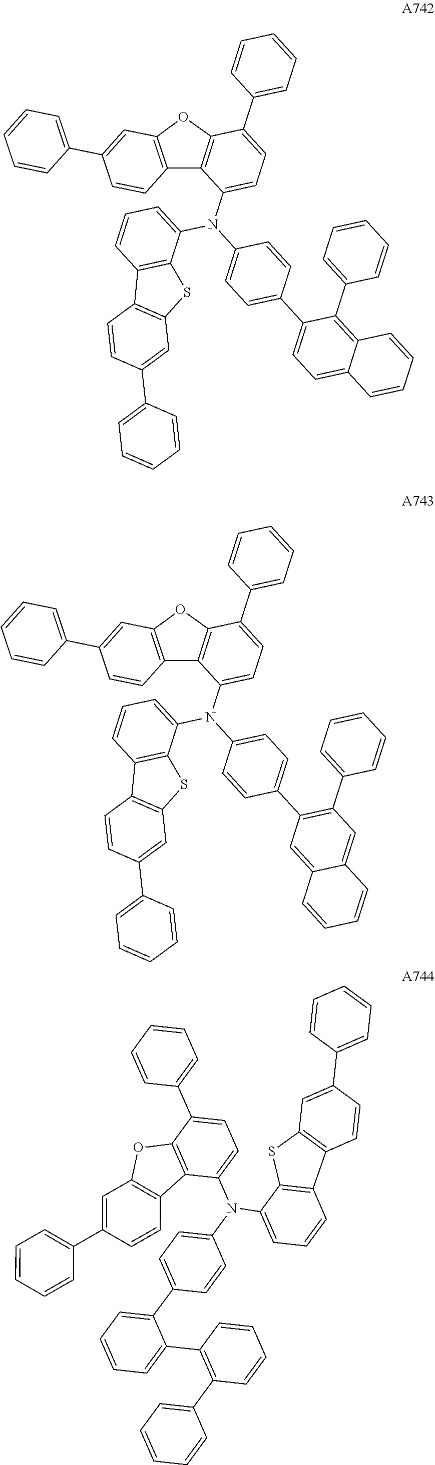 [SMILES: c1ccc(-c2ccc3c(c2)oc2c(-c4ccccc4)ccc(N(c4ccc(-c5ccc6ccccc6c5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5cc6ccccc6cc5-c5ccccc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccccc5-c5ccccc5-c5ccccc5)cc4)c4cccc5c4sc4cc(-c6ccccc6)ccc45)c23)cc1]

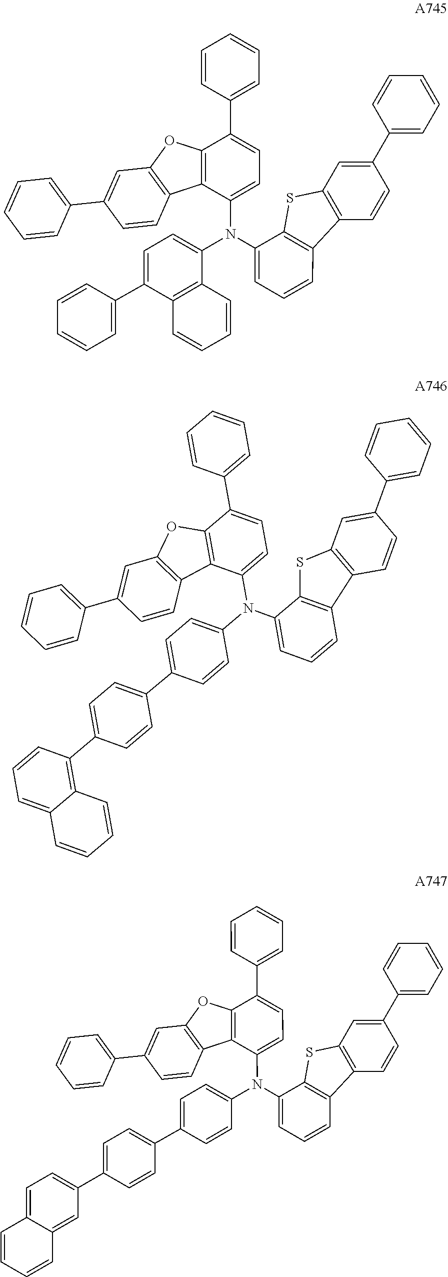 [SMILES: c1ccc(-c2ccc3c(c2)oc2c(-c4ccccc4)ccc(N(c4ccc(-c5ccccc5)c5ccccc45)c4cccc5c4sc4cc(-c6ccccc6)ccc45)c23)cc1 ; c1ccc(-c2ccc3c(c2)oc2c(-c4ccccc4)ccc(N(c4ccc(-c5ccc(-c6cccc7ccccc67)cc5)cc4)c4cccc5c4sc4cc(-c6ccccc6)ccc45)c23)cc1 ; c1ccc(-c2ccc3c(c2)oc2c(-c4ccccc4)ccc(N(c4ccc(-c5ccc(-c6ccc7ccccc7c6)cc5)cc4)c4cccc5c4sc4cc(-c6ccccc6)ccc45)c23)cc1]

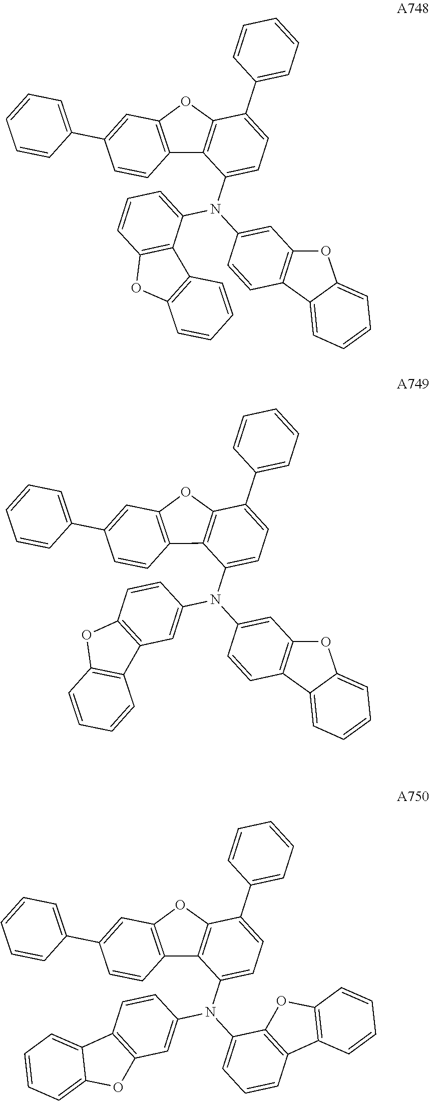 [SMILES: c1ccc(-c2ccc3c(c2)oc2c(-c4ccccc4)ccc(N(c4ccc5c(c4)oc4ccccc45)c4cccc5oc6ccccc6c45)c23)cc1 ; c1ccc(-c2ccc3c(c2)oc2c(-c4ccccc4)ccc(N(c4ccc5c(c4)oc4ccccc45)c4ccc5oc6ccccc6c5c4)c23)cc1 ; c1ccc(-c2ccc3c(c2)oc2c(-c4ccccc4)ccc(N(c4ccc5c(c4)oc4ccccc45)c4cccc5c4oc4ccccc45)c23)cc1]

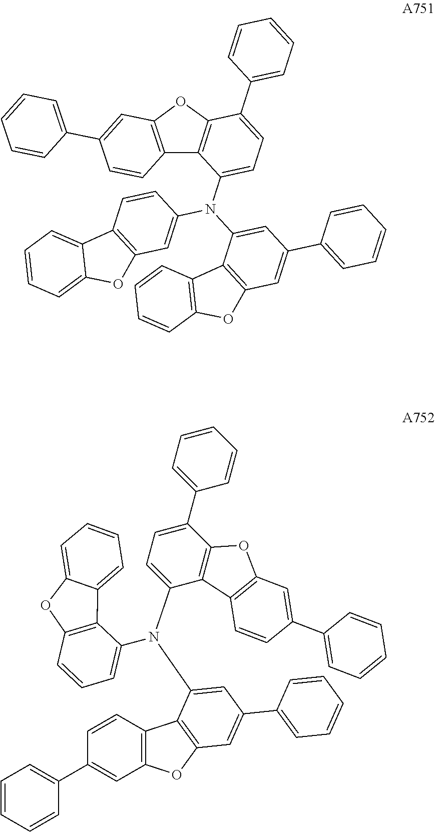 [SMILES: c1ccc(-c2ccc3c(c2)oc2c(-c4ccccc4)ccc(N(c4ccc5c(c4)oc4ccccc45)c4cc(-c5ccccc5)cc5oc6ccccc6c45)c23)cc1 ; c1ccc(-c2ccc3c(c2)oc2cc(-c4ccccc4)cc(N(c4cccc5oc6ccccc6c45)c4ccc(-c5ccccc5)c5oc6cc(-c7ccccc7)ccc6c45)c23)cc1]

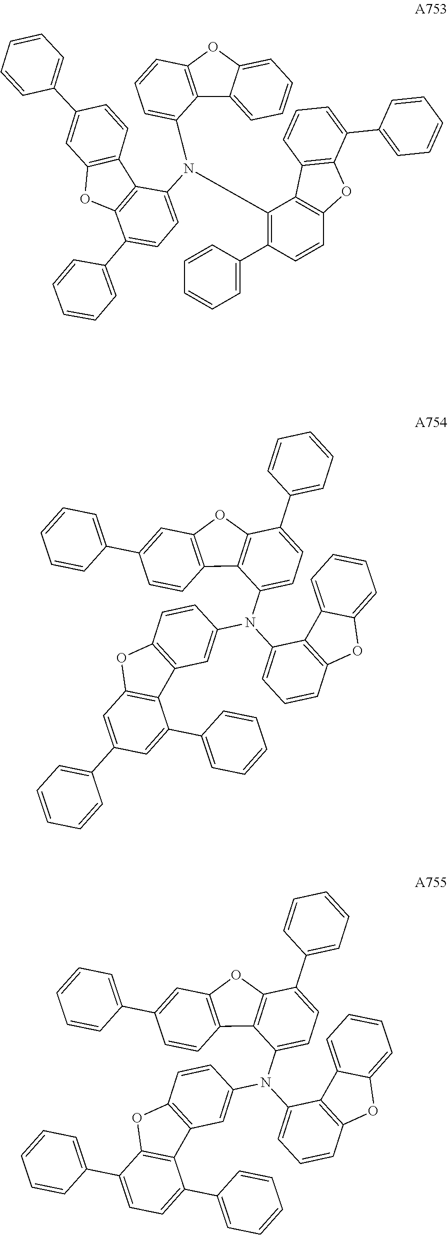 [SMILES: c1ccc(-c2ccc3c(c2)oc2c(-c4ccccc4)ccc(N(c4cccc5oc6ccccc6c45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5oc6ccccc6c45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5oc6ccccc6c45)c23)cc1]

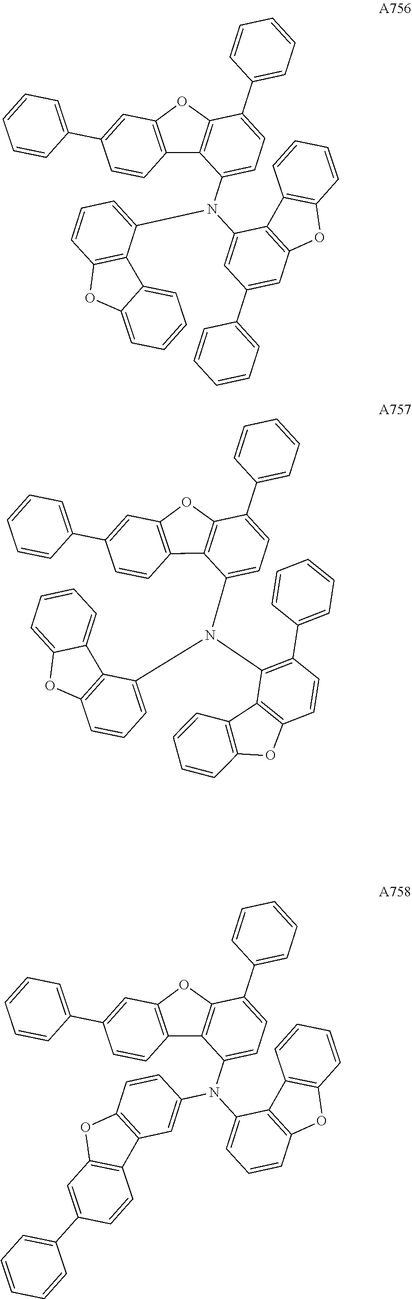 [SMILES: c1ccc(-c2ccc3c(c2)oc2c(-c4ccccc4)ccc(N(c4cccc5oc6ccccc6c45)c4cc(-c5ccccc5)cc5oc6ccccc6c45)c23)cc1 ; c1ccc(-c2ccc3c(c2)oc2c(-c4ccccc4)ccc(N(c4cccc5oc6ccccc6c45)c4c(-c5ccccc5)ccc5oc6ccccc6c45)c23)cc1 ; c1ccc(-c2ccc3c(c2)oc2ccc(N(c4cccc5oc6ccccc6c45)c4ccc(-c5ccccc5)c5oc6cc(-c7ccccc7)ccc6c45)cc23)cc1]

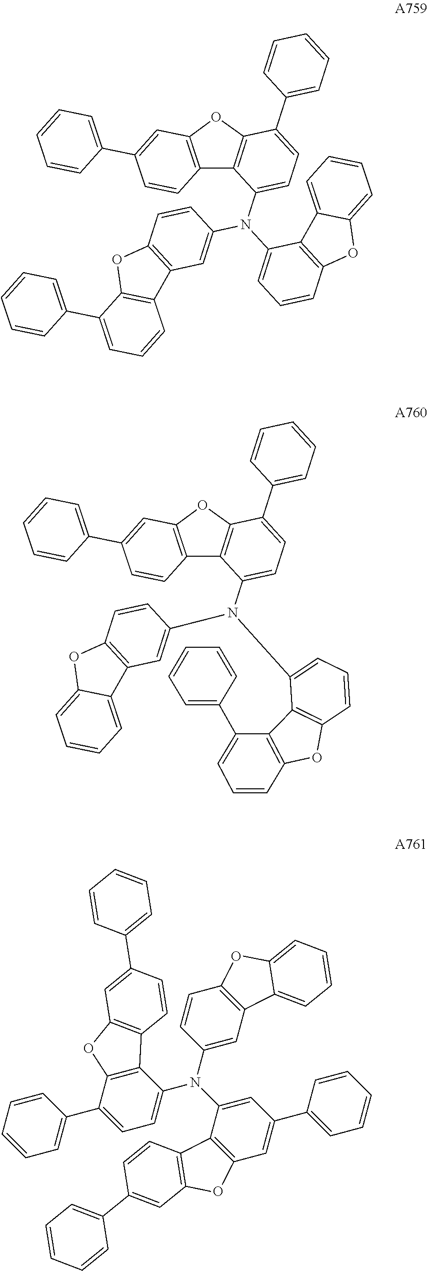 [SMILES: c1ccc(-c2ccc3c(c2)oc2c(-c4ccccc4)ccc(N(c4ccc5oc6c(-c7ccccc7)cccc6c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5oc6ccccc6c5c4)c4ccc(-c5ccccc5)c5oc6cc(-c7ccccc7)ccc6c45)c23)cc1]

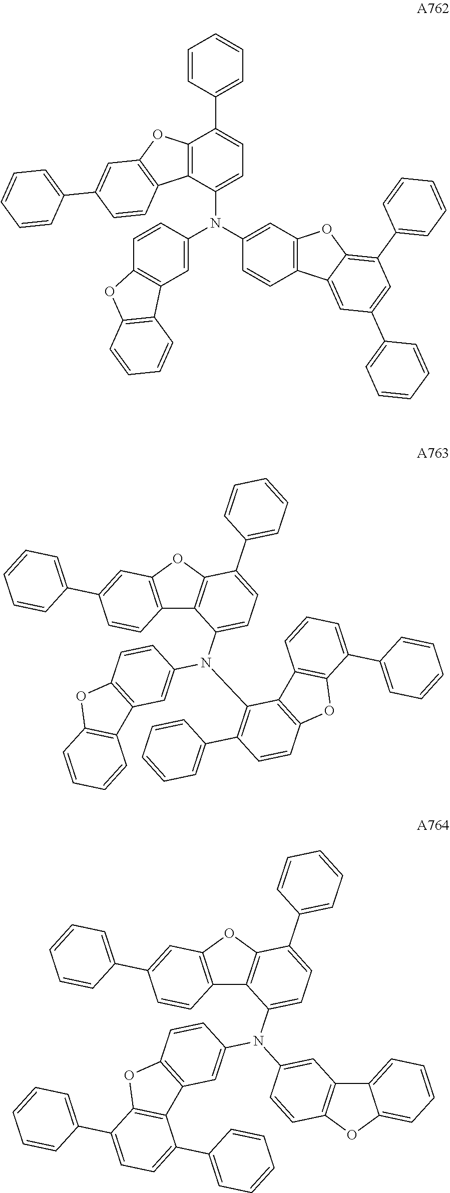 [SMILES: c1ccc(-c2ccc3c(c2)oc2c(-c4ccccc4)ccc(N(c4ccc5c(c4)oc4c(-c6ccccc6)cc(-c6ccccc6)cc45)c4ccc5oc6ccccc6c5c4)c23)cc1 ; c1ccc(-c2ccc3c(c2)oc2c(-c4ccccc4)ccc(N(c4ccc5oc6ccccc6c5c4)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6ccccc6c5c4)c4ccc5oc6c(-c7ccccc7)ccc(-c7ccccc7)c6c5c4)c23)cc1]

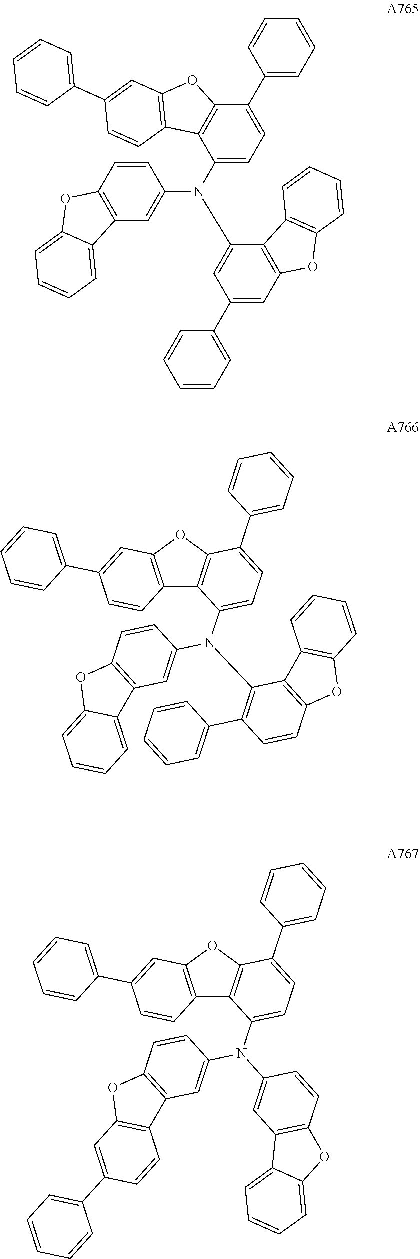 [SMILES: c1ccc(-c2ccc3c(c2)oc2c(-c4ccccc4)ccc(N(c4ccc5oc6ccccc6c5c4)c4cc(-c5ccccc5)cc5oc6ccccc6c45)c23)cc1 ; c1ccc(-c2ccc3c(c2)oc2c(-c4ccccc4)ccc(N(c4ccc5oc6ccccc6c5c4)c4c(-c5ccccc5)ccc5oc6ccccc6c45)c23)cc1 ; c1ccc(-c2ccc3c(c2)oc2ccc(N(c4ccc5oc6ccccc6c5c4)c4ccc(-c5ccccc5)c5oc6cc(-c7ccccc7)ccc6c45)cc23)cc1]

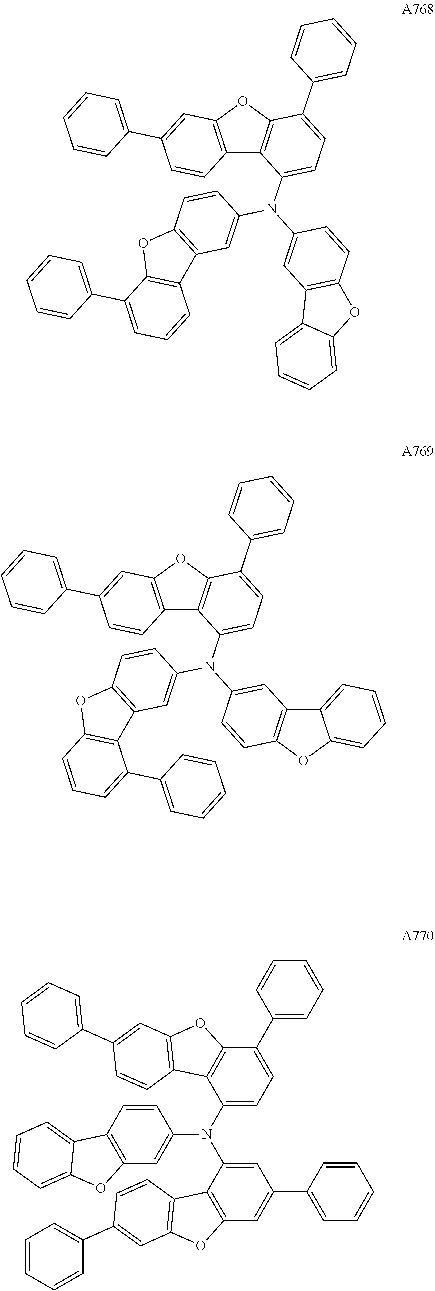 [SMILES: c1ccc(-c2ccc3c(c2)oc2c(-c4ccccc4)ccc(N(c4ccc5oc6ccccc6c5c4)c4ccc5oc6c(-c7ccccc7)cccc6c5c4)c23)cc1 ; c1ccc(-c2ccc3c(c2)oc2c(-c4ccccc4)ccc(N(c4ccc5oc6ccccc6c5c4)c4ccc5oc6cccc(-c7ccccc7)c6c5c4)c23)cc1 ; c1ccc(-c2ccc3c(c2)oc2cc(-c4ccccc4)cc(N(c4ccc5c(c4)oc4ccccc45)c4ccc(-c5ccccc5)c5oc6cc(-c7ccccc7)ccc6c45)c23)cc1]

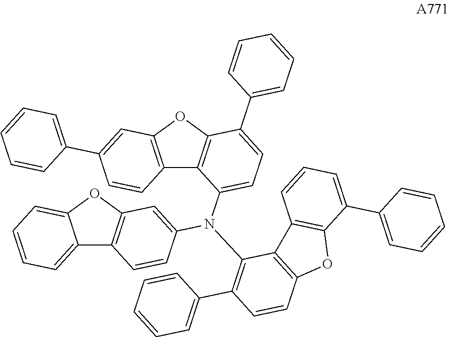 [SMILES: c1ccc(-c2ccc3c(c2)oc2c(-c4ccccc4)ccc(N(c4ccc5c(c4)oc4ccccc45)c4c(-c5ccccc5)ccc5oc6c(-c7ccccc7)cccc6c45)c23)cc1]

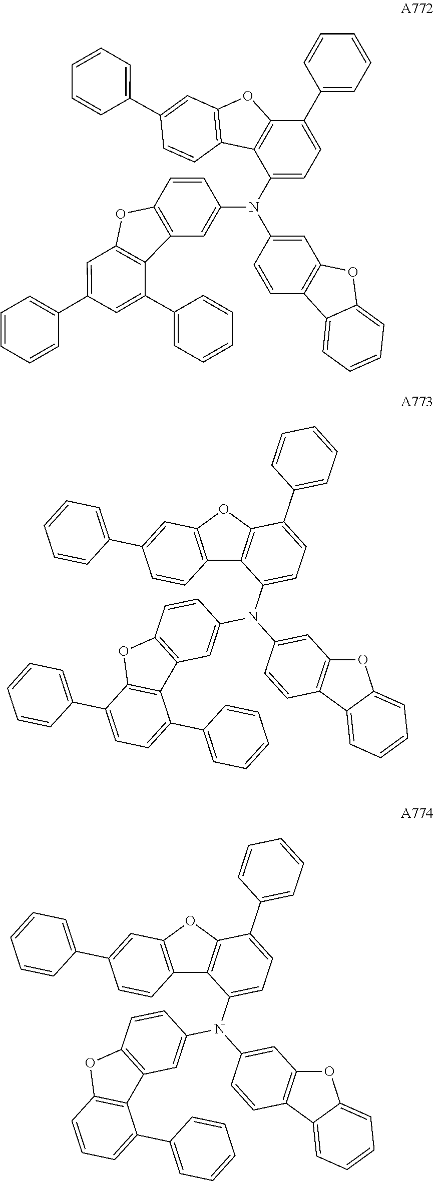 [SMILES: c1ccc(-c2ccc3c(c2)oc2c(-c4ccccc4)ccc(N(c4ccc5c(c4)oc4ccccc45)c4ccc5oc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5oc6c(-c7ccccc7)ccc(-c7ccccc7)c6c5c4)c23)cc1 ; c1ccc(-c2ccc3c(c2)oc2c(-c4ccccc4)ccc(N(c4ccc5c(c4)oc4ccccc45)c4ccc5oc6cccc(-c7ccccc7)c6c5c4)c23)cc1]

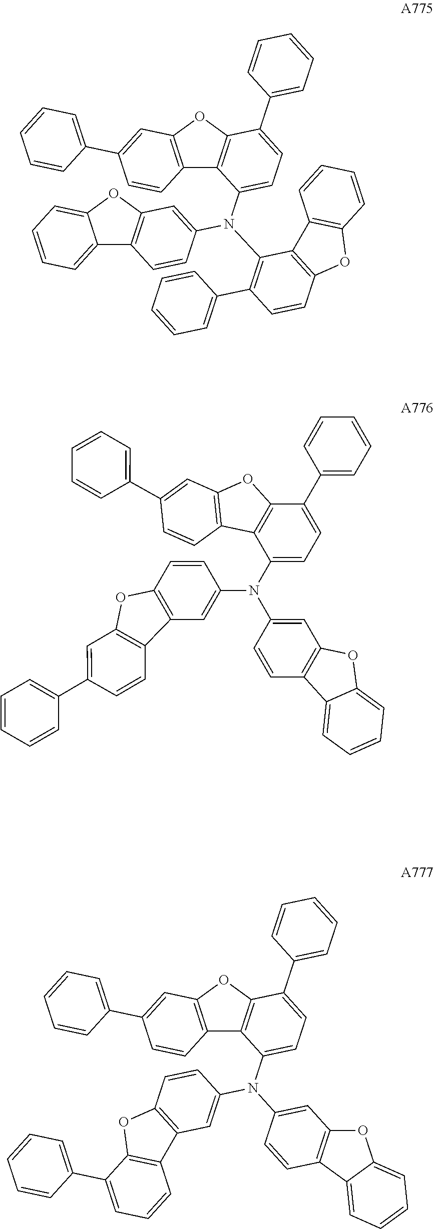 [SMILES: c1ccc(-c2ccc3c(c2)oc2c(-c4ccccc4)ccc(N(c4ccc5c(c4)oc4ccccc45)c4c(-c5ccccc5)ccc5oc6ccccc6c45)c23)cc1 ; c1ccc(-c2ccc3c(c2)oc2ccc(N(c4ccc5c(c4)oc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5c(c4)oc4ccccc45)c4ccc5oc6c(-c7ccccc7)cccc6c5c4)c23)cc1]

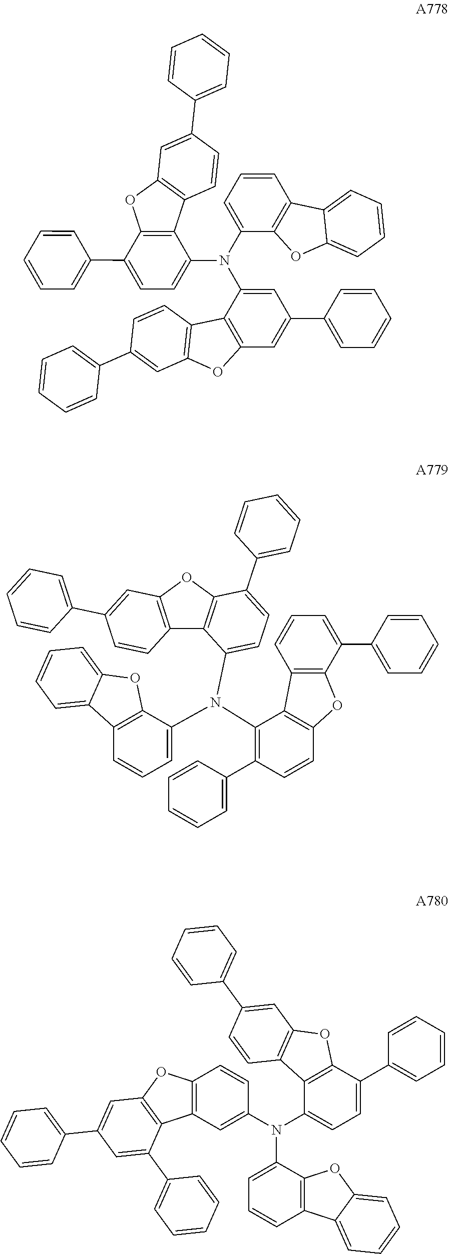 [SMILES: c1ccc(-c2ccc3c(c2)oc2cc(-c4ccccc4)cc(N(c4cccc5c4oc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4cccc5c4oc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5c4oc4ccccc45)c23)cc1]

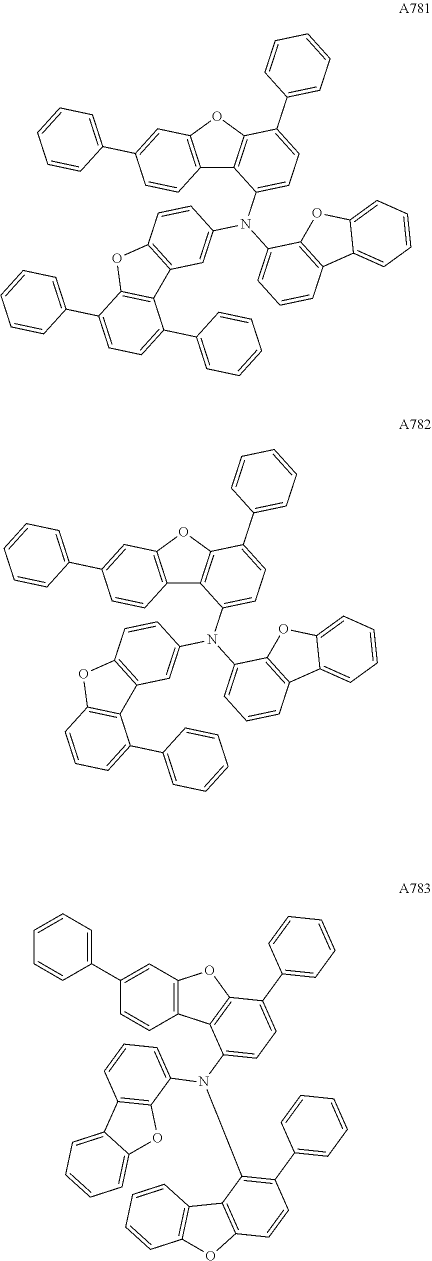 [SMILES: c1ccc(-c2ccc3c(c2)oc2c(-c4ccccc4)ccc(N(c4ccc5oc6c(-c7ccccc7)ccc(-c7ccccc7)c6c5c4)c4cccc5c4oc4ccccc45)c23)cc1 ; c1ccc(-c2ccc3c(c2)oc2c(-c4ccccc4)ccc(N(c4ccc5oc6cccc(-c7ccccc7)c6c5c4)c4cccc5c4oc4ccccc45)c23)cc1 ; c1ccc(-c2ccc3c(c2)oc2c(-c4ccccc4)ccc(N(c4cccc5c4oc4ccccc45)c4c(-c5ccccc5)ccc5oc6ccccc6c45)c23)cc1]

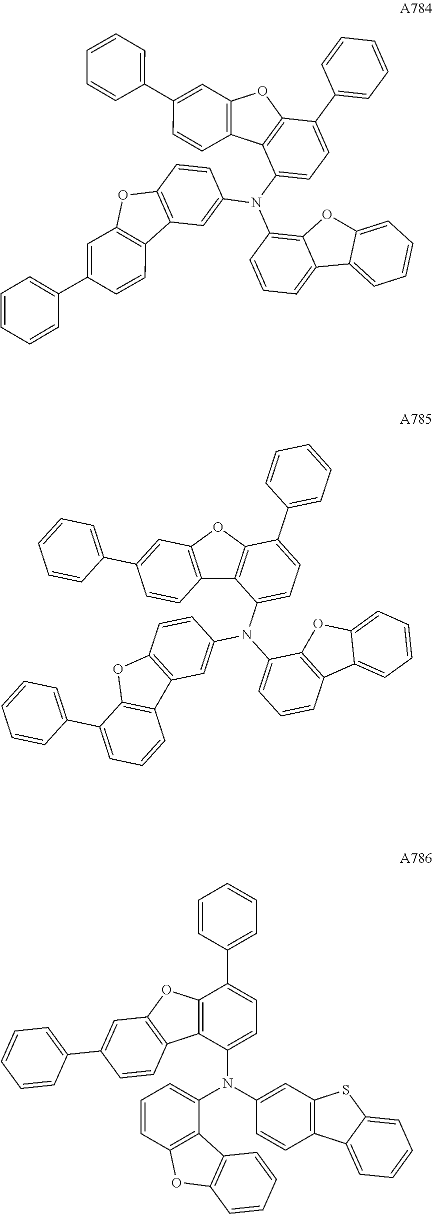 [SMILES: c1ccc(-c2ccc3c(c2)oc2ccc(N(c4cccc5c4oc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4cccc5c4oc4ccccc45)c23)cc1 ; c1ccc(-c2ccc3c(c2)oc2c(-c4ccccc4)ccc(N(c4ccc5c(c4)sc4ccccc45)c4cccc5oc6ccccc6c45)c23)cc1]

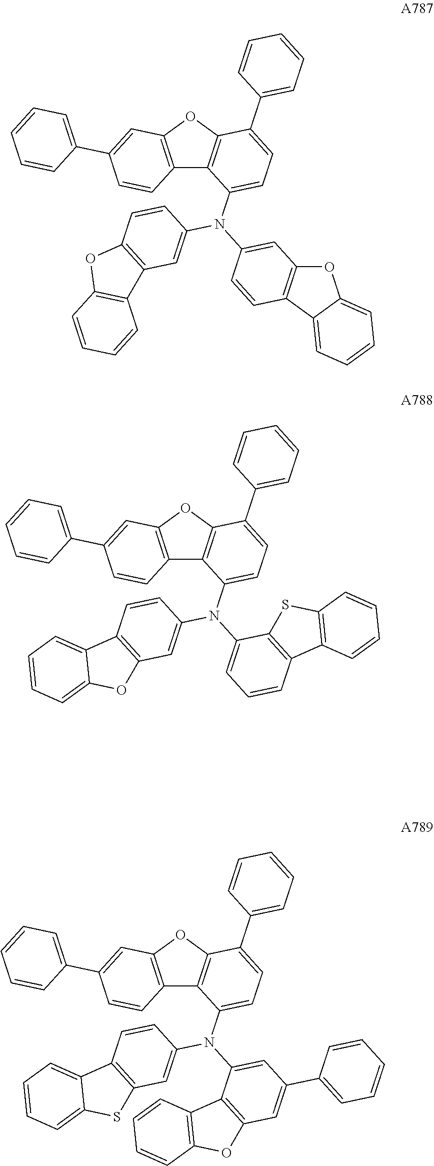 [SMILES: c1ccc(-c2ccc3c(c2)oc2c(-c4ccccc4)ccc(N(c4ccc5c(c4)oc4ccccc45)c4ccc5oc6ccccc6c5c4)c23)cc1 ; c1ccc(-c2ccc3c(c2)oc2c(-c4ccccc4)ccc(N(c4ccc5c(c4)oc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5oc6ccccc6c45)c23)cc1]

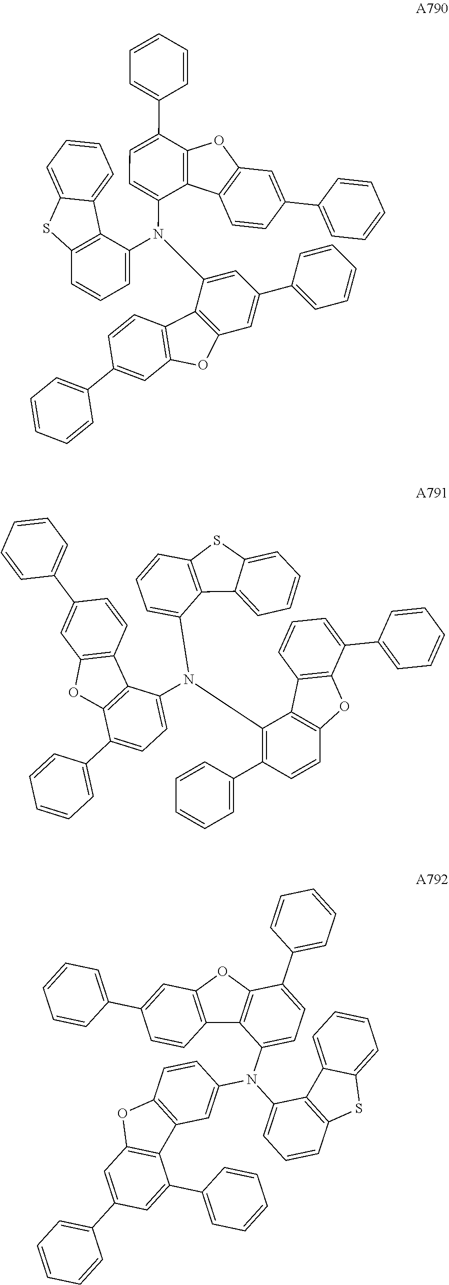 [SMILES: c1ccc(-c2ccc3c(c2)oc2cc(-c4ccccc4)cc(N(c4cccc5sc6ccccc6c45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4c(-c5ccccc5)ccc5oc6c(-c7ccccc7)cccc6c45)c4cccc5sc6ccccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5sc6ccccc6c45)c23)cc1]

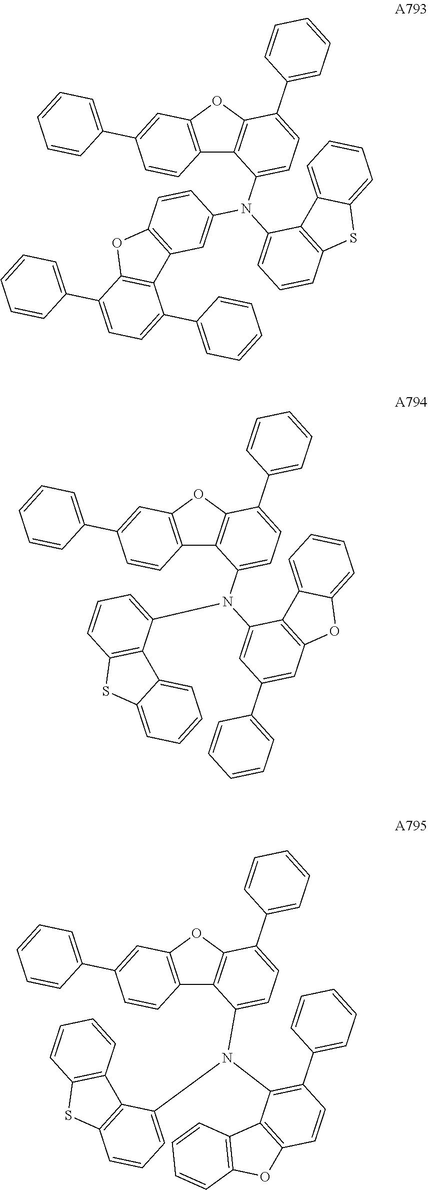 [SMILES: c1ccc(-c2ccc3c(c2)oc2c(-c4ccccc4)ccc(N(c4ccc5oc6c(-c7ccccc7)ccc(-c7ccccc7)c6c5c4)c4cccc5sc6ccccc6c45)c23)cc1 ; c1ccc(-c2ccc3c(c2)oc2c(-c4ccccc4)ccc(N(c4cc(-c5ccccc5)cc5oc6ccccc6c45)c4cccc5sc6ccccc6c45)c23)cc1 ; c1ccc(-c2ccc3c(c2)oc2c(-c4ccccc4)ccc(N(c4c(-c5ccccc5)ccc5oc6ccccc6c45)c4cccc5sc6ccccc6c45)c23)cc1]

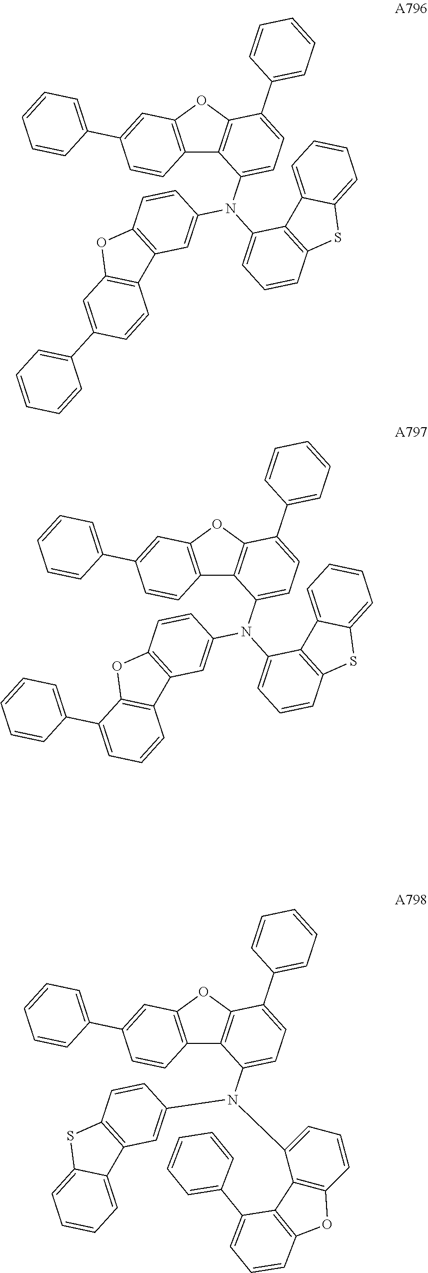 [SMILES: c1ccc(-c2ccc3c(c2)oc2ccc(N(c4cccc5sc6ccccc6c45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5oc6cccc(-c7ccccc7)c6c45)c23)cc1]

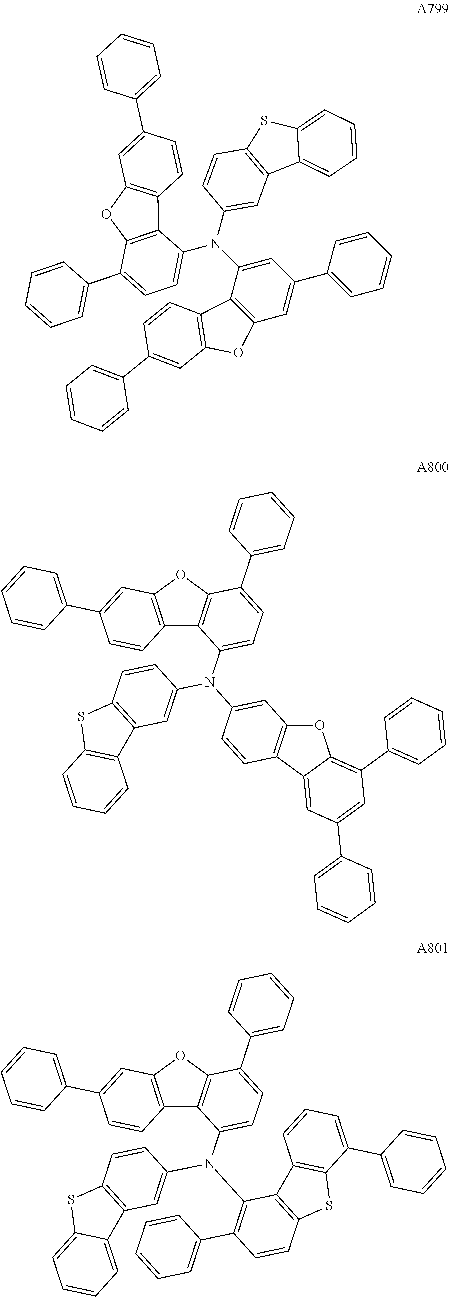

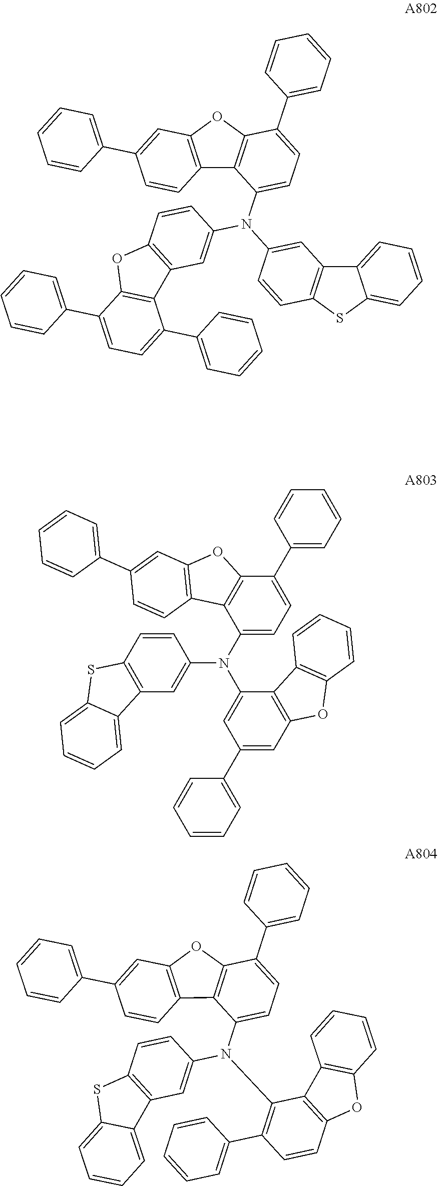

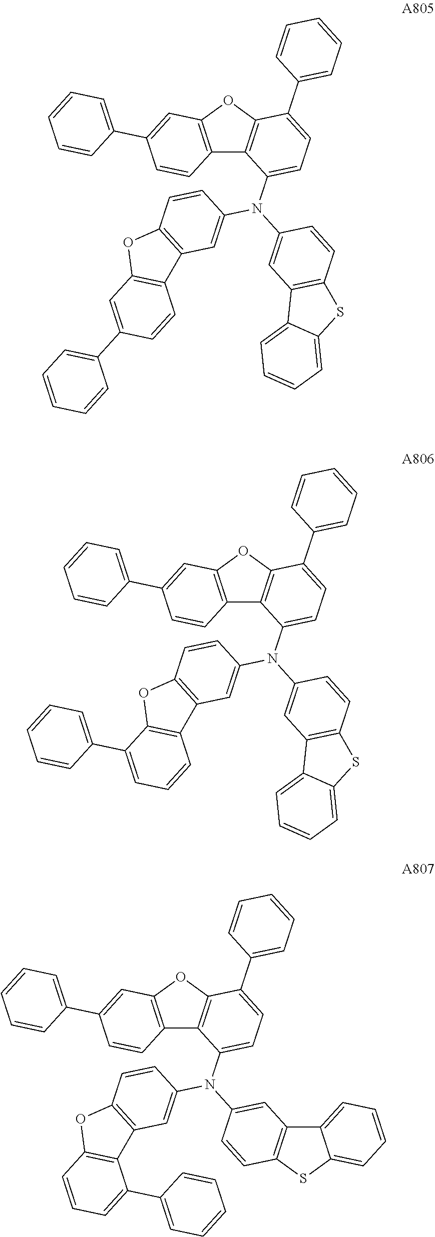 [SMILES: c1ccc(-c2ccc3c(c2)oc2ccc(N(c4ccc5sc6ccccc6c5c4)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5oc6c(-c7ccccc7)cccc6c5c4)c4ccc5sc6ccccc6c5c4)c23)cc1 ; c1ccc(-c2ccc3c(c2)oc2c(-c4ccccc4)ccc(N(c4ccc5sc6ccccc6c5c4)c4ccc5oc6cccc(-c7ccccc7)c6c5c4)c23)cc1]

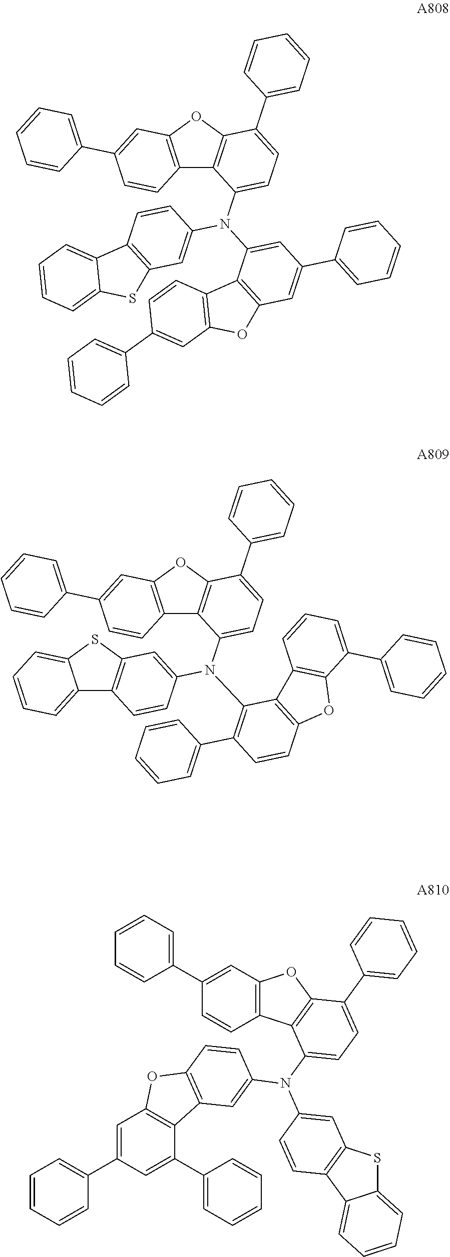 [SMILES: c1ccc(-c2ccc3c(c2)oc2cc(-c4ccccc4)cc(N(c4ccc5c(c4)sc4ccccc45)c4ccc(-c5ccccc5)c5oc6cc(-c7ccccc7)ccc6c45)c23)cc1 ; c1ccc(-c2ccc3c(c2)oc2c(-c4ccccc4)ccc(N(c4ccc5c(c4)sc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5oc6cc(-c7ccccc7)cc(-c7ccccc7)c6c5c4)c23)cc1]

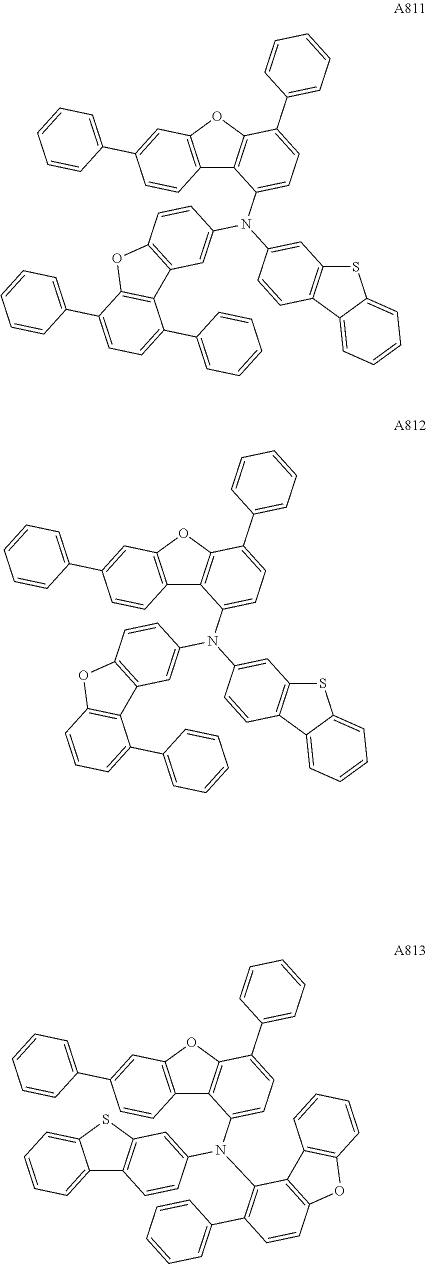 [SMILES: c1ccc(-c2ccc3c(c2)oc2c(-c4ccccc4)ccc(N(c4ccc5c(c4)sc4ccccc45)c4ccc5oc6c(-c7ccccc7)ccc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5oc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5oc6ccccc6c45)c23)cc1]

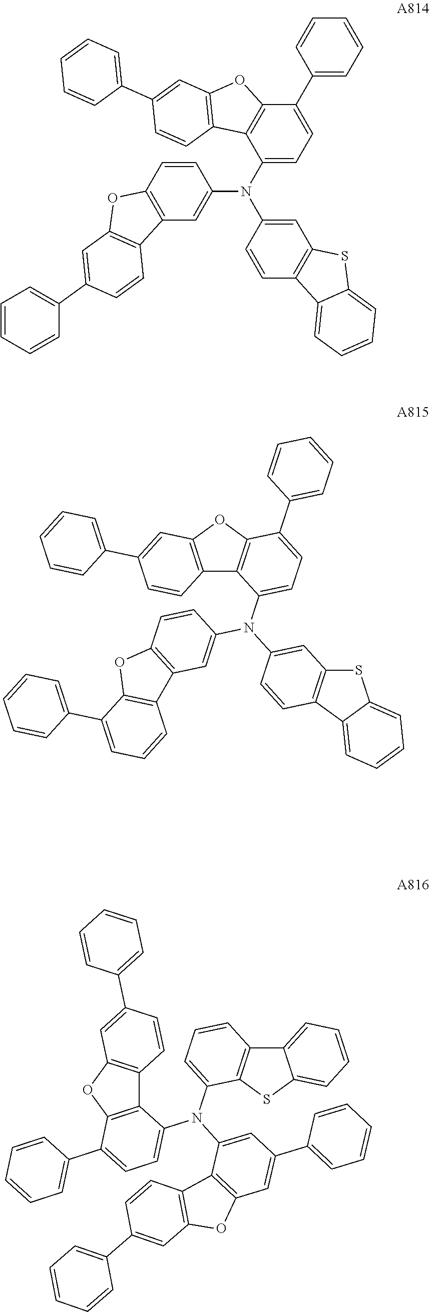 [SMILES: c1ccc(-c2ccc3c(c2)oc2ccc(N(c4ccc5c(c4)sc4ccccc45)c4ccc(-c5ccccc5)c5oc6cc(-c7ccccc7)ccc6c45)cc23)cc1 ; c1ccc(-c2ccc3c(c2)oc2c(-c4ccccc4)ccc(N(c4ccc5c(c4)sc4ccccc45)c4ccc5oc6c(-c7ccccc7)cccc6c5c4)c23)cc1 ; c1ccc(-c2ccc3c(c2)oc2cc(-c4ccccc4)cc(N(c4cccc5c4sc4ccccc45)c4ccc(-c5ccccc5)c5oc6cc(-c7ccccc7)ccc6c45)c23)cc1]

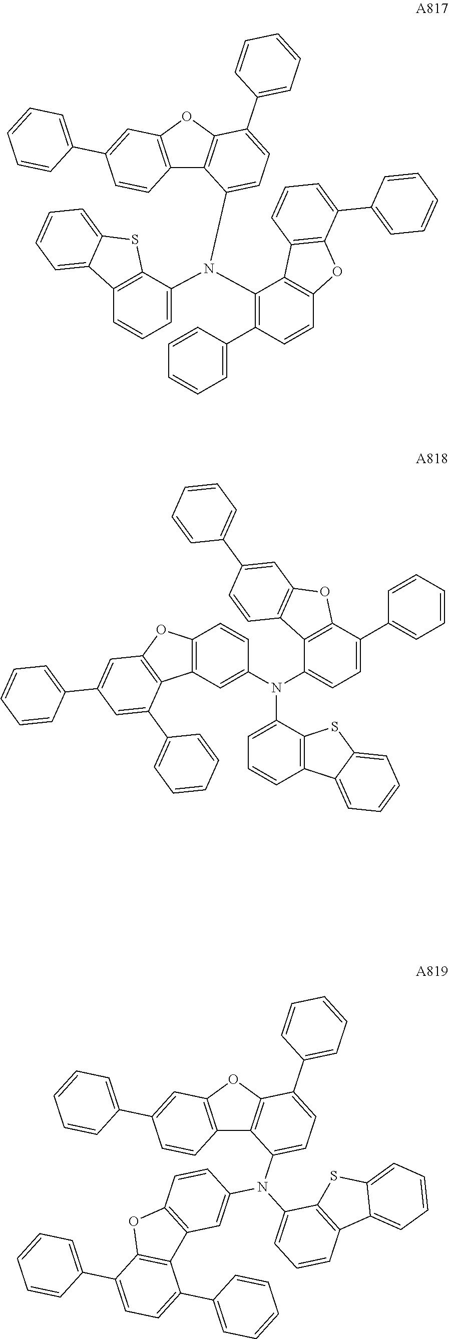 [SMILES: c1ccc(-c2ccc3c(c2)oc2c(-c4ccccc4)ccc(N(c4cccc5c4sc4ccccc45)c4c(-c5ccccc5)ccc5oc6c(-c7ccccc7)cccc6c45)c23)cc1 ; c1ccc(-c2ccc3c(c2)oc2c(-c4ccccc4)ccc(N(c4ccc5oc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5oc6c(-c7ccccc7)ccc(-c7ccccc7)c6c5c4)c4cccc5c4sc4ccccc45)c23)cc1]

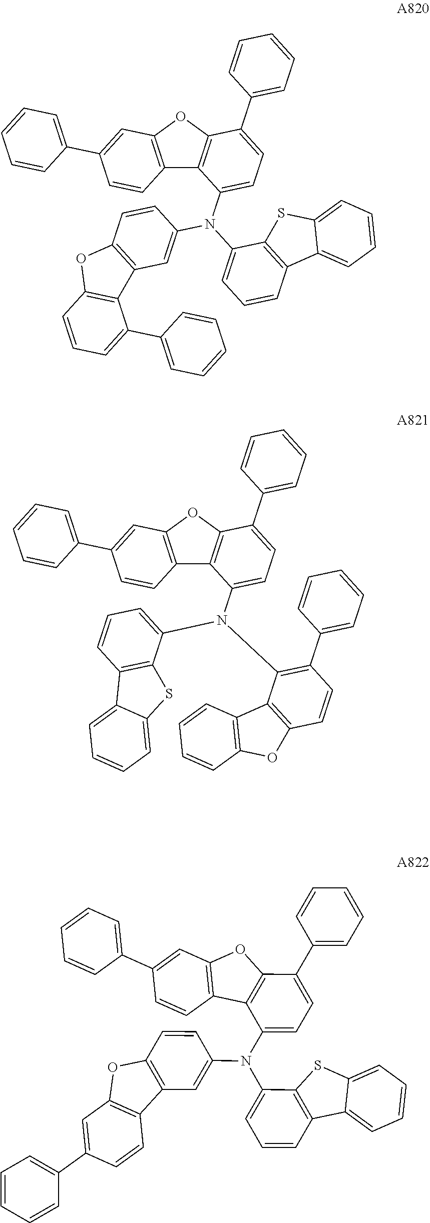 [SMILES: c1ccc(-c2ccc3c(c2)oc2c(-c4ccccc4)ccc(N(c4ccc5oc6cccc(-c7ccccc7)c6c5c4)c4cccc5c4sc4ccccc45)c23)cc1 ; c1ccc(-c2ccc3c(c2)oc2c(-c4ccccc4)ccc(N(c4cccc5c4sc4ccccc45)c4c(-c5ccccc5)ccc5oc6ccccc6c45)c23)cc1 ; c1ccc(-c2ccc3c(c2)oc2ccc(N(c4cccc5c4sc4ccccc45)c4ccc(-c5ccccc5)c5oc6cc(-c7ccccc7)ccc6c45)cc23)cc1]

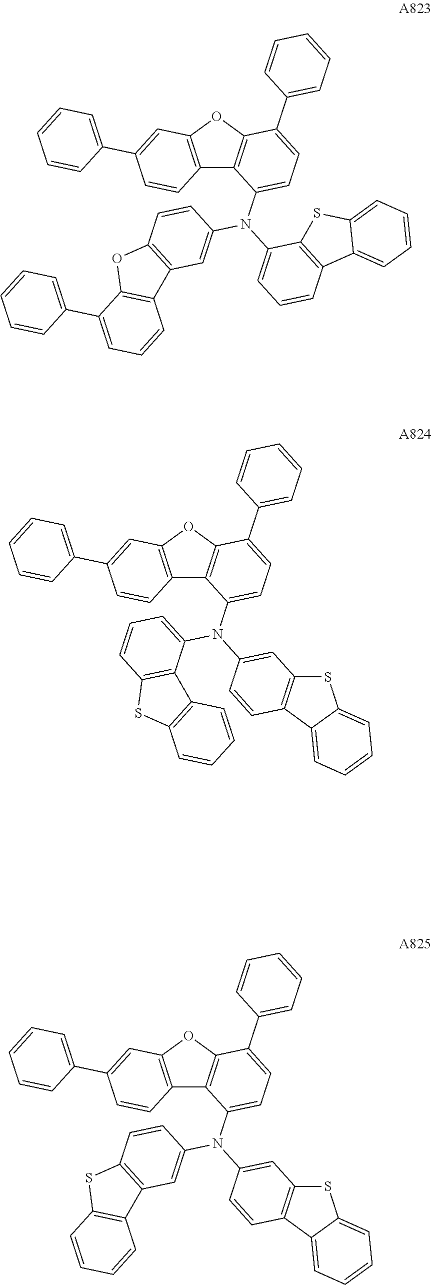 [SMILES: c1ccc(-c2ccc3c(c2)oc2c(-c4ccccc4)ccc(N(c4ccc5oc6c(-c7ccccc7)cccc6c5c4)c4cccc5c4sc4ccccc45)c23)cc1 ; c1ccc(-c2ccc3c(c2)oc2c(-c4ccccc4)ccc(N(c4ccc5c(c4)sc4ccccc45)c4cccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6ccccc6c5c4)c23)cc1]

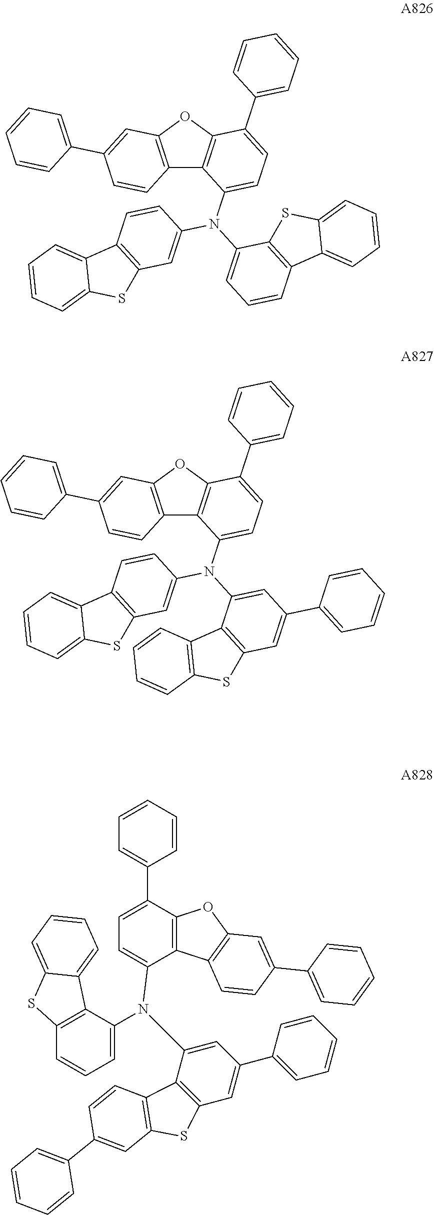 [SMILES: c1ccc(-c2ccc3c(c2)oc2c(-c4ccccc4)ccc(N(c4ccc5c(c4)sc4ccccc45)c4cccc5c4sc4ccccc45)c23)cc1 ; c1ccc(-c2ccc3c(c2)oc2c(-c4ccccc4)ccc(N(c4ccc5c(c4)sc4ccccc45)c4cc(-c5ccccc5)cc5sc6ccccc6c45)c23)cc1 ; c1ccc(-c2ccc3c(c2)oc2c(-c4ccccc4)ccc(N(c4cccc5sc6ccccc6c45)c4cc(-c5ccccc5)cc5sc6cc(-c7ccccc7)ccc6c45)c23)cc1]

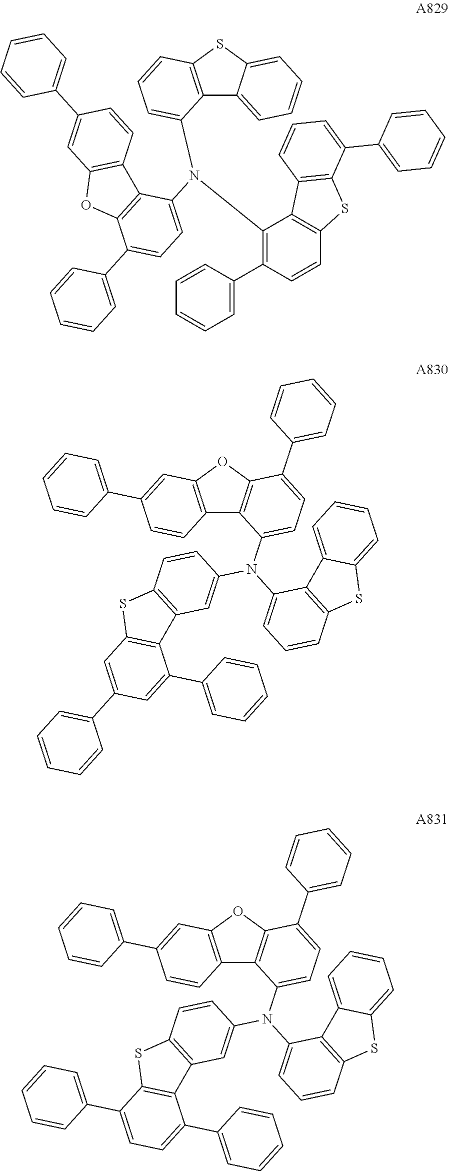 [SMILES: c1ccc(-c2ccc3c(c2)oc2c(-c4ccccc4)ccc(N(c4cccc5sc6ccccc6c45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6cc(-c7ccccc7)cc(-c7ccccc7)c6c5c4)c4cccc5sc6ccccc6c45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5sc6ccccc6c45)c23)cc1]

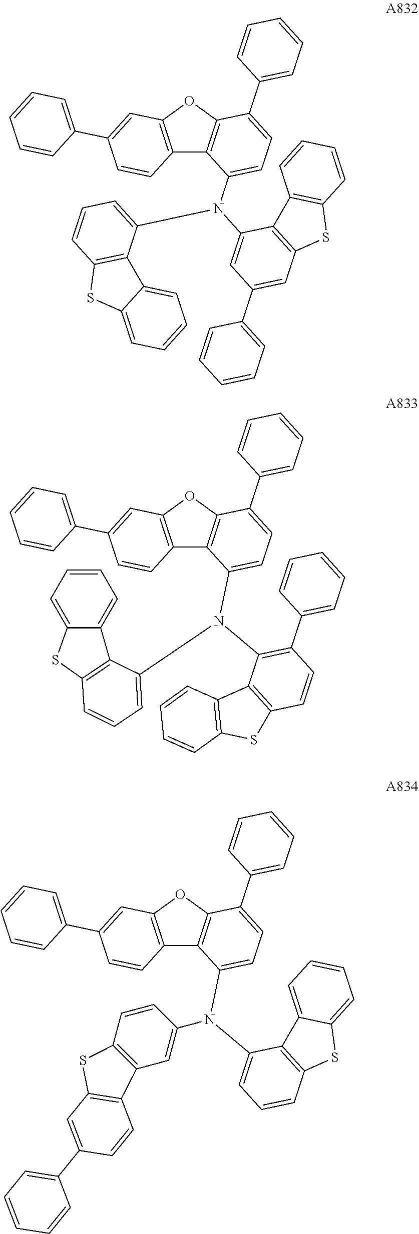

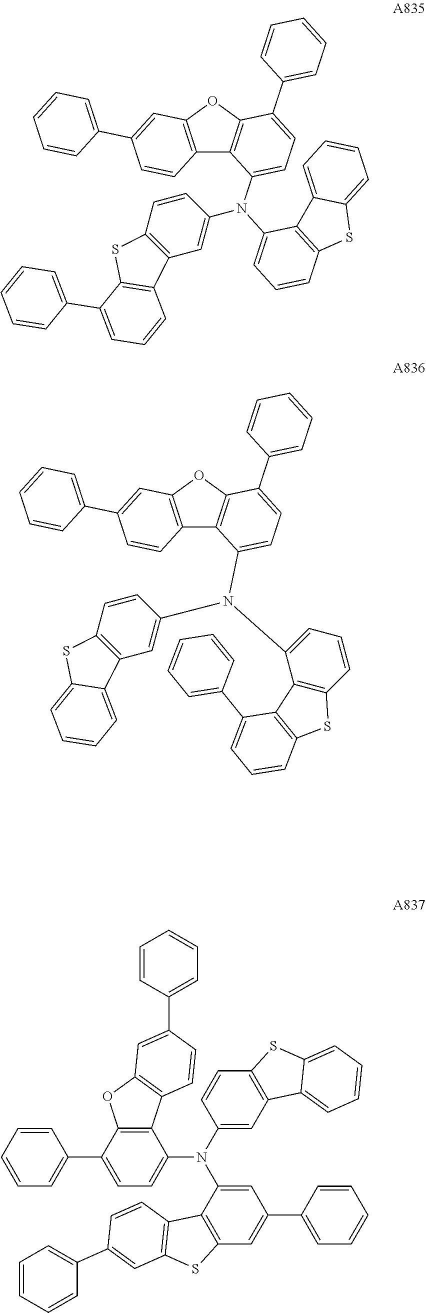 [SMILES: c1ccc(-c2ccc3c(c2)oc2c(-c4ccccc4)ccc(N(c4ccc5sc6c(-c7ccccc7)cccc6c5c4)c4cccc5sc6ccccc6c45)c23)cc1 ; c1ccc(-c2ccc3c(c2)oc2c(-c4ccccc4)ccc(N(c4ccc5sc6ccccc6c5c4)c4cccc5sc6cccc(-c7ccccc7)c6c45)c23)cc1 ; c1ccc(-c2ccc3c(c2)oc2c(-c4ccccc4)ccc(N(c4ccc5sc6ccccc6c5c4)c4cc(-c5ccccc5)cc5sc6cc(-c7ccccc7)ccc6c45)c23)cc1]

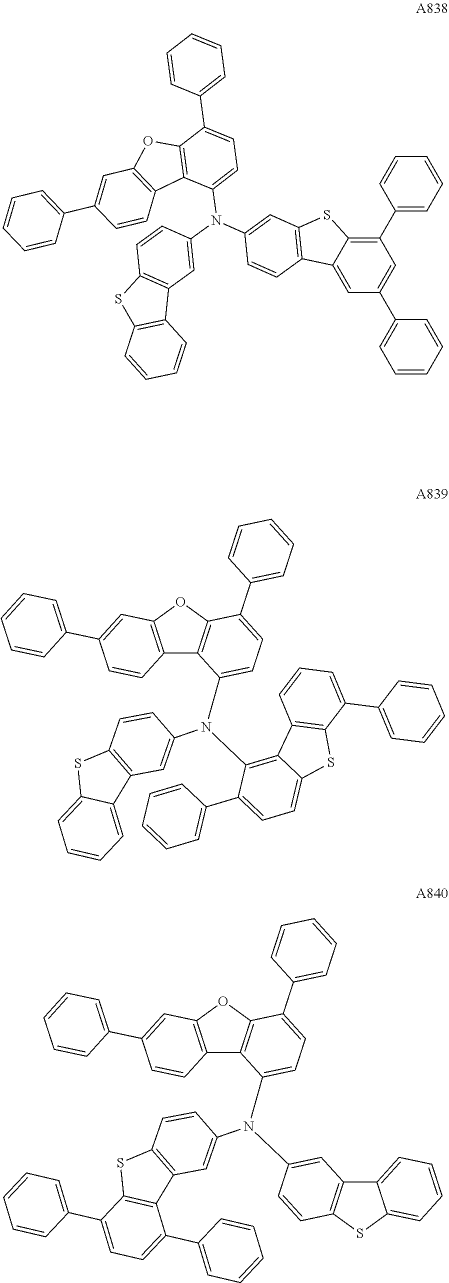 [SMILES: c1ccc(-c2ccc3c(c2)oc2c(-c4ccccc4)ccc(N(c4ccc5c(c4)sc4c(-c6ccccc6)cc(-c6ccccc6)cc45)c4ccc5sc6ccccc6c5c4)c23)cc1 ; c1ccc(-c2ccc3c(c2)oc2c(-c4ccccc4)ccc(N(c4ccc5sc6ccccc6c5c4)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6ccccc6c5c4)c4ccc5sc6c(-c7ccccc7)ccc(-c7ccccc7)c6c5c4)c23)cc1]

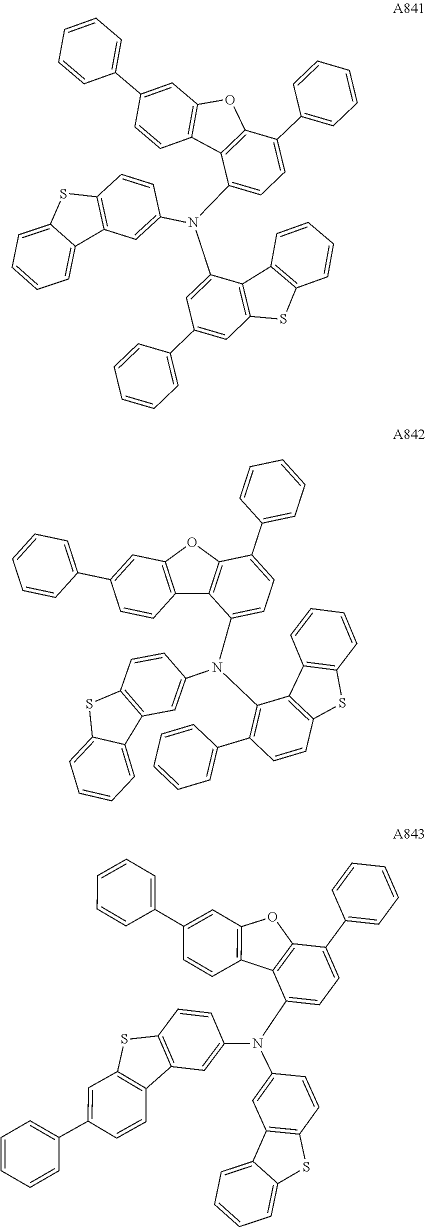 [SMILES: c1ccc(-c2ccc3c(c2)oc2c(-c4ccccc4)ccc(N(c4ccc5sc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc5sc6ccccc6c5c4)c4c(-c5ccccc5)ccc5sc6ccccc6c45)c23)cc1 ; c1ccc(-c2ccc3c(c2)oc2c(-c4ccccc4)ccc(N(c4ccc5sc6ccccc6c5c4)c4ccc5sc6cc(-c7ccccc7)ccc6c5c4)c23)cc1]

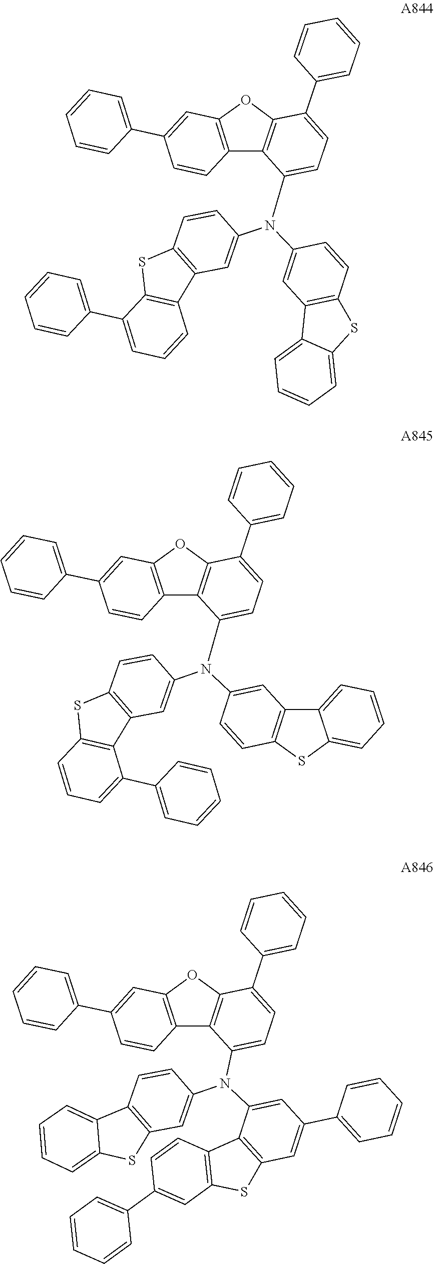 [SMILES: c1ccc(-c2ccc3c(c2)oc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)oc2c(-c4ccccc4)ccc(N(c4ccc5sc6ccccc6c5c4)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4cc(-c5ccccc5)cc5sc6cc(-c7ccccc7)ccc6c45)c23)cc1]

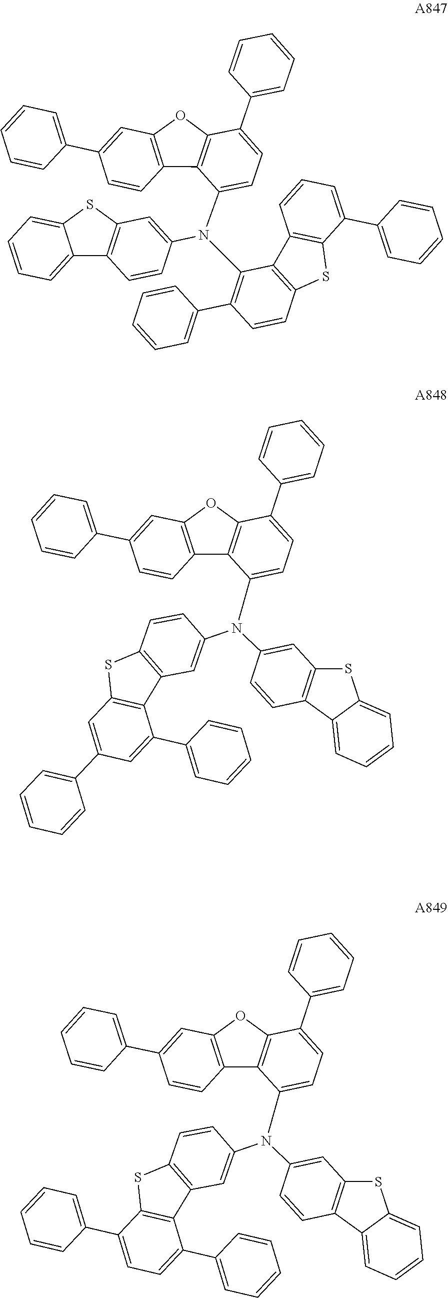 [SMILES: c1ccc(-c2ccc3c(c2)oc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)cc(-c7ccccc7)c6c5c4)c23)cc1 ; c1ccc(-c2ccc3c(c2)oc2c(-c4ccccc4)ccc(N(c4ccc5c(c4)sc4ccccc45)c4ccc5sc6c(-c7ccccc7)ccc(-c7ccccc7)c6c5c4)c23)cc1]

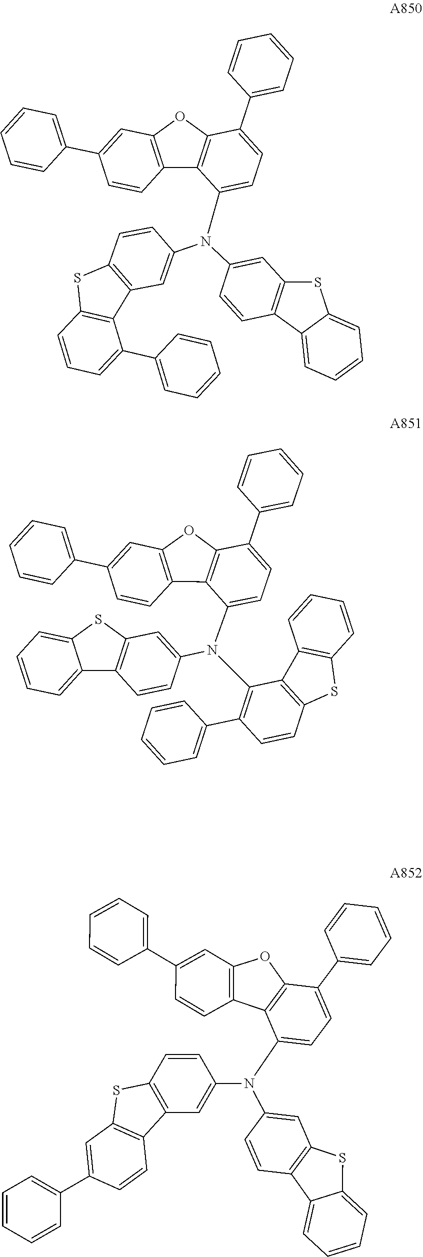 [SMILES: c1ccc(-c2ccc3c(c2)oc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)oc2c(-c4ccccc4)ccc(N(c4ccc5c(c4)sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5c(c4)sc4ccccc45)c4ccc5sc6cc(-c7ccccc7)ccc6c5c4)c23)cc1]

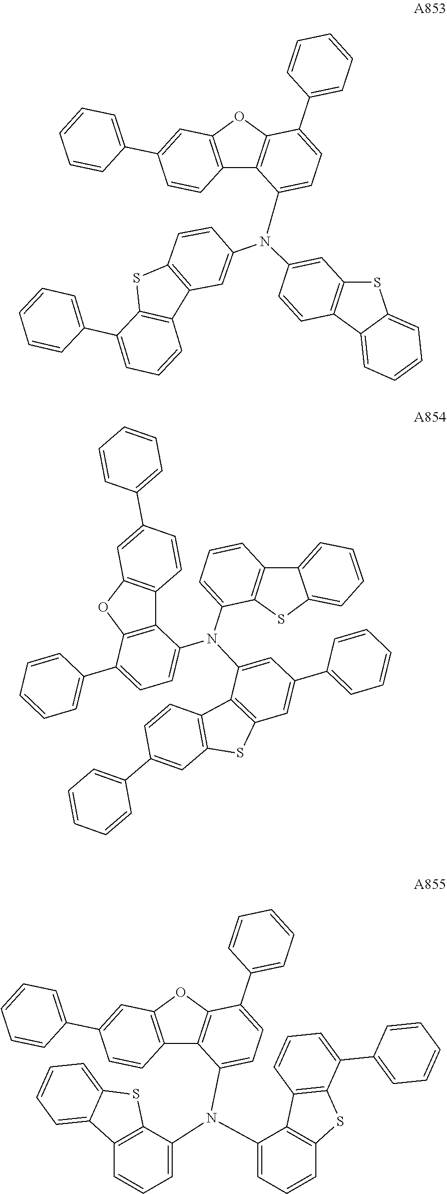 [SMILES: c1ccc(-c2ccc3c(c2)oc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)oc2c(-c4ccccc4)ccc(N(c4cccc5c4sc4ccccc45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5c4sc4ccccc45)c4cccc5sc6c(-c7ccccc7)cccc6c45)c23)cc1]

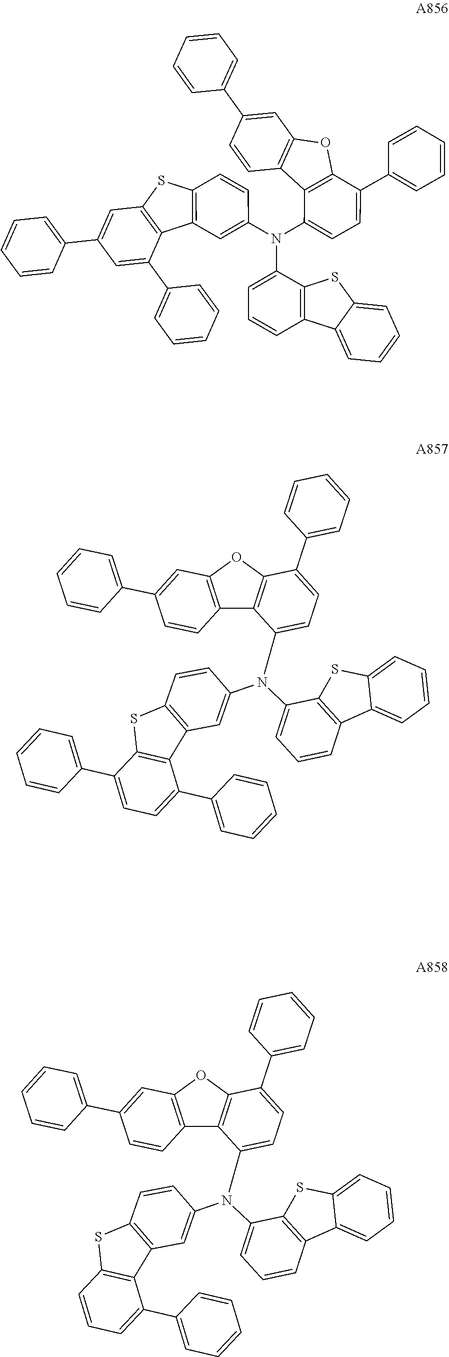 [SMILES: c1ccc(-c2ccc3c(c2)oc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5c4sc4ccccc45)c23)cc1 ; c1ccc(-c2ccc3c(c2)oc2c(-c4ccccc4)ccc(N(c4ccc5sc6cccc(-c7ccccc7)c6c5c4)c4cccc5c4sc4ccccc45)c23)cc1]

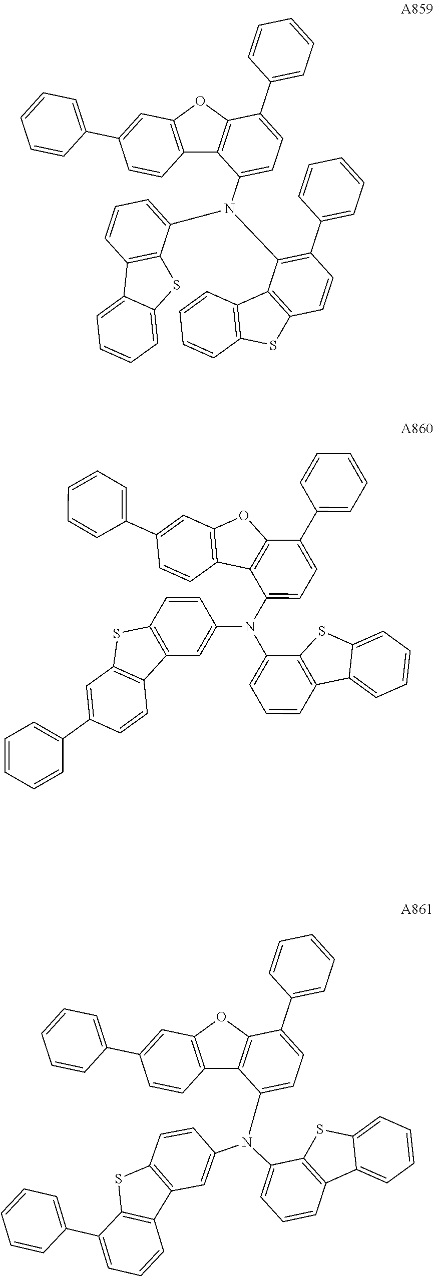 [SMILES: c1ccc(-c2ccc3c(c2)oc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)oc2c(-c4ccccc4)ccc(N(c4ccc5sc6cc(-c7ccccc7)ccc6c5c4)c4cccc5c4sc4ccccc45)c23)cc1 ; c1ccc(-c2ccc3c(c2)oc2c(-c4ccccc4)ccc(N(c4ccc5sc6c(-c7ccccc7)cccc6c5c4)c4cccc5c4sc4ccccc45)c23)cc1]

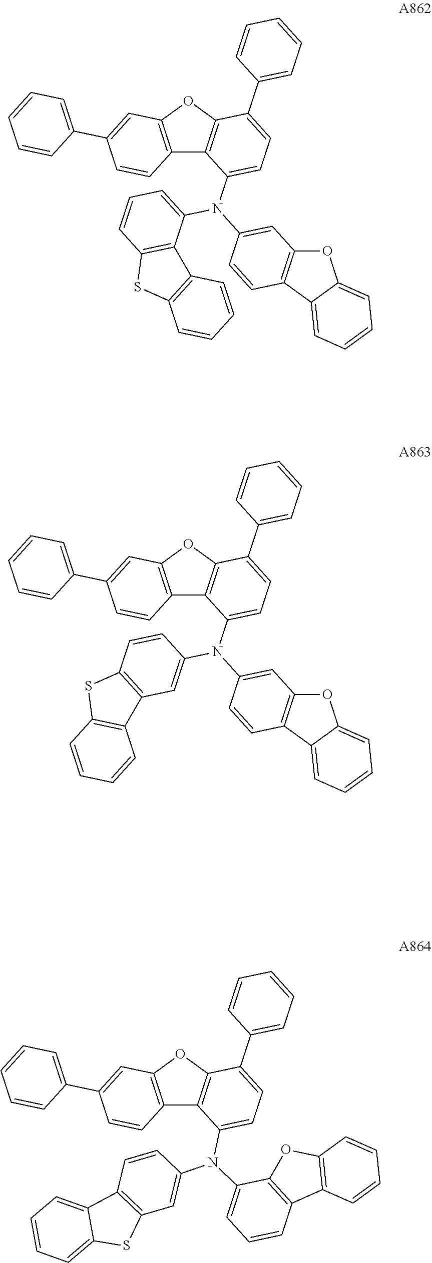 [SMILES: c1ccc(-c2ccc3c(c2)oc2c(-c4ccccc4)ccc(N(c4ccc5c(c4)oc4ccccc45)c4cccc5sc6ccccc6c45)c23)cc1 ; c1ccc(-c2ccc3c(c2)oc2c(-c4ccccc4)ccc(N(c4ccc5c(c4)oc4ccccc45)c4ccc5sc6ccccc6c5c4)c23)cc1 ; c1ccc(-c2ccc3c(c2)oc2c(-c4ccccc4)ccc(N(c4ccc5c(c4)sc4ccccc45)c4cccc5c4oc4ccccc45)c23)cc1]

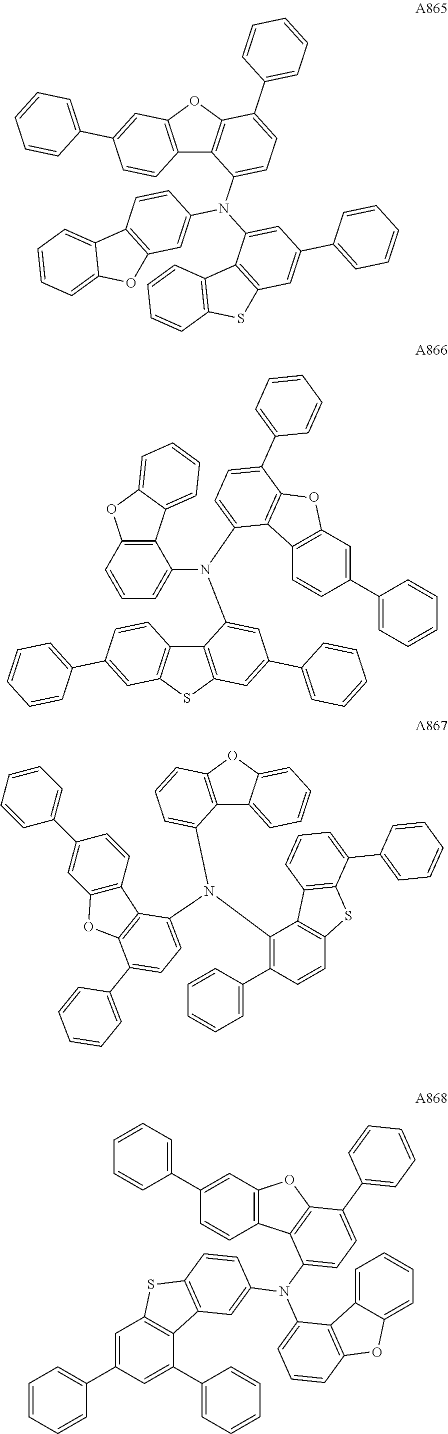 [SMILES: c1ccc(-c2ccc3c(c2)oc2c(-c4ccccc4)ccc(N(c4ccc5c(c4)oc4ccccc45)c4cc(-c5ccccc5)cc5sc6ccccc6c45)c23)cc1 ; c1ccc(-c2ccc3c(c2)oc2c(-c4ccccc4)ccc(N(c4cccc5oc6ccccc6c45)c4cc(-c5ccccc5)cc5sc6cc(-c7ccccc7)ccc6c45)c23)cc1 ; c1ccc(-c2ccc3c(c2)oc2c(-c4ccccc4)ccc(N(c4cccc5oc6ccccc6c45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6cc(-c7ccccc7)cc(-c7ccccc7)c6c5c4)c4cccc5oc6ccccc6c45)c23)cc1]

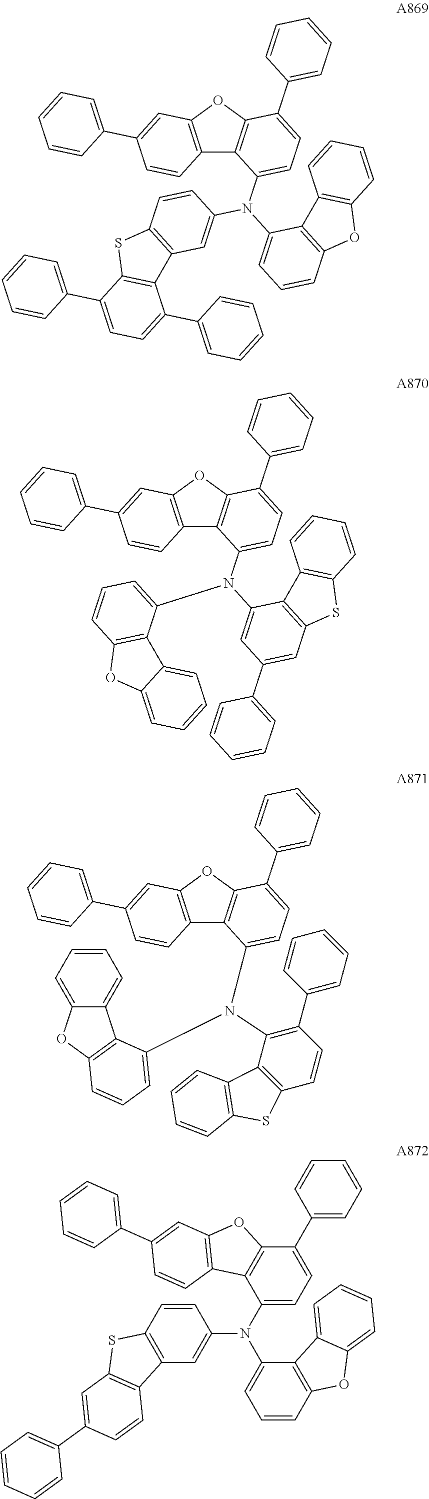

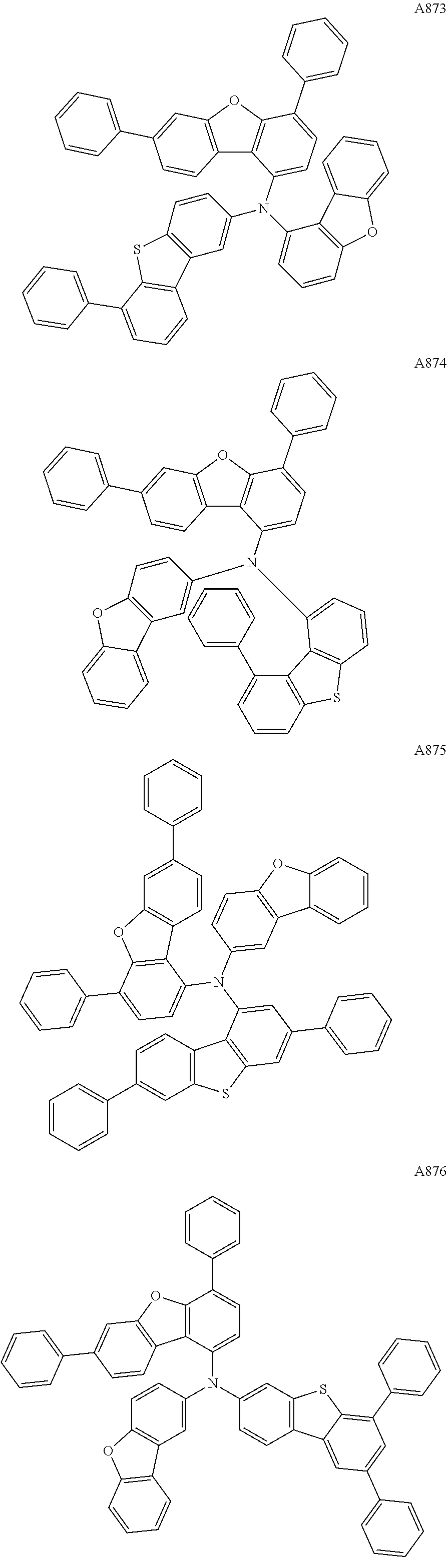 [SMILES: c1ccc(-c2ccc3c(c2)oc2c(-c4ccccc4)ccc(N(c4ccc5sc6c(-c7ccccc7)cccc6c5c4)c4cccc5oc6ccccc6c45)c23)cc1 ; c1ccc(-c2ccc3c(c2)oc2c(-c4ccccc4)ccc(N(c4ccc5oc6ccccc6c5c4)c4cccc5sc6cccc(-c7ccccc7)c6c45)c23)cc1 ; c1ccc(-c2ccc3c(c2)oc2c(-c4ccccc4)ccc(N(c4ccc5oc6ccccc6c5c4)c4cc(-c5ccccc5)cc5sc6cc(-c7ccccc7)ccc6c45)c23)cc1 ; c1ccc(-c2ccc3c(c2)oc2c(-c4ccccc4)ccc(N(c4ccc5c(c4)sc4c(-c6ccccc6)cc(-c6ccccc6)cc45)c4ccc5oc6ccccc6c5c4)c23)cc1]

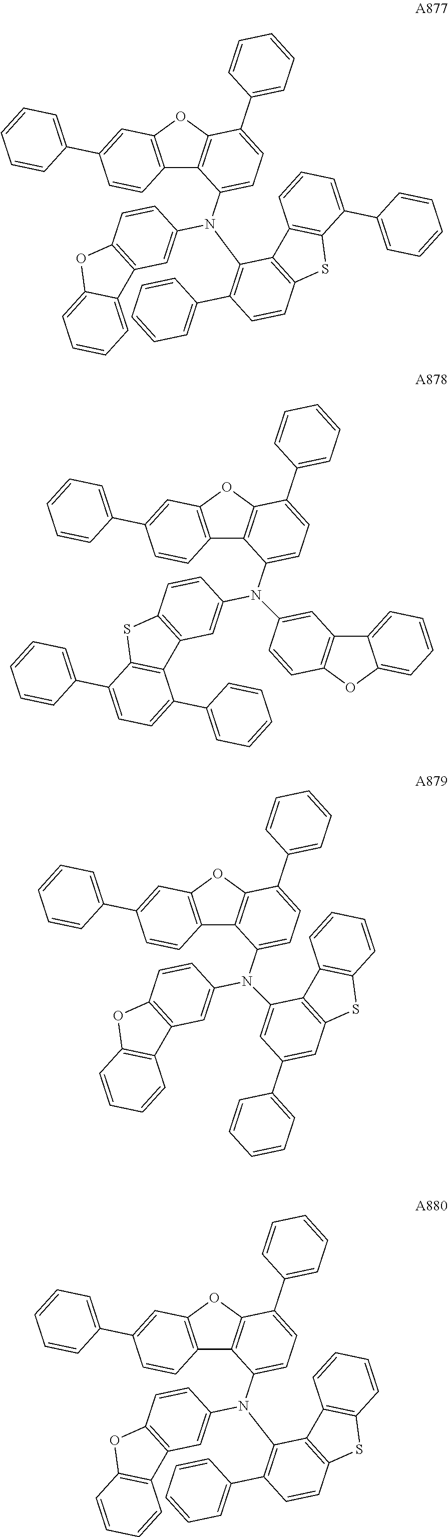 [SMILES: c1ccc(-c2ccc3c(c2)oc2c(-c4ccccc4)ccc(N(c4ccc5oc6ccccc6c5c4)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5oc6ccccc6c5c4)c4ccc5sc6c(-c7ccccc7)ccc(-c7ccccc7)c6c5c4)c23)cc1 ; c1ccc(-c2ccc3c(c2)oc2c(-c4ccccc4)ccc(N(c4ccc5oc6ccccc6c5c4)c4cc(-c5ccccc5)cc5sc6ccccc6c45)c23)cc1 ; c1ccc(-c2ccc3c(c2)oc2c(-c4ccccc4)ccc(N(c4ccc5oc6ccccc6c5c4)c4c(-c5ccccc5)ccc5sc6ccccc6c45)c23)cc1]

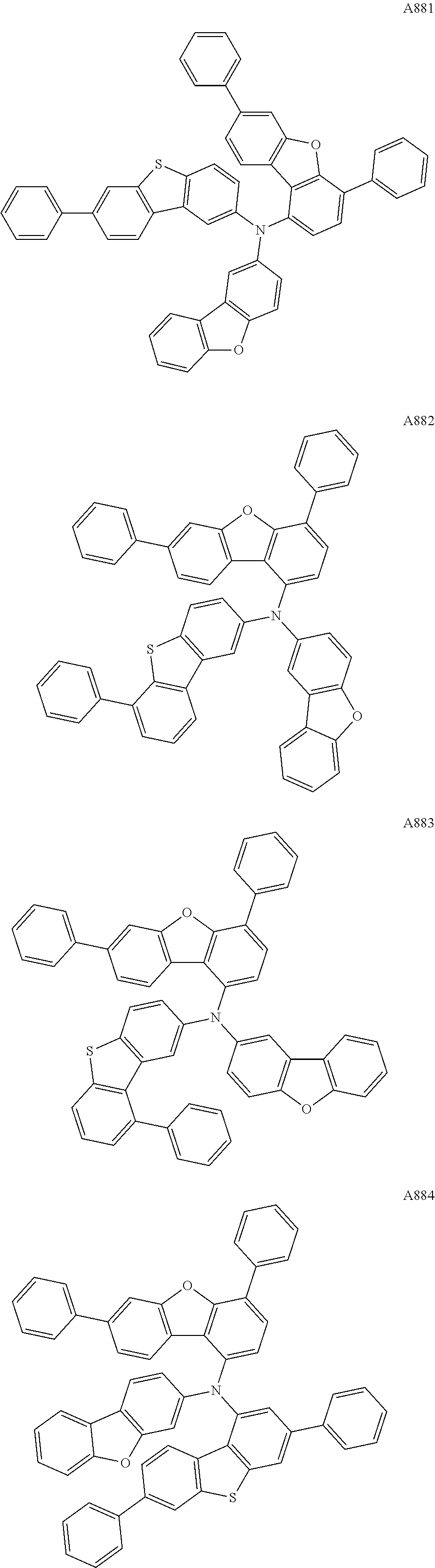 [SMILES: c1ccc(-c2ccc3c(c2)oc2c(-c4ccccc4)ccc(N(c4ccc5oc6ccccc6c5c4)c4ccc5sc6cc(-c7ccccc7)ccc6c5c4)c23)cc1 ; c1ccc(-c2ccc3c(c2)oc2c(-c4ccccc4)ccc(N(c4ccc5oc6ccccc6c5c4)c4ccc5sc6c(-c7ccccc7)cccc6c5c4)c23)cc1 ; c1ccc(-c2ccc3c(c2)oc2c(-c4ccccc4)ccc(N(c4ccc5oc6ccccc6c5c4)c4ccc5sc6cccc(-c7ccccc7)c6c5c4)c23)cc1 ; c1ccc(-c2ccc3c(c2)oc2c(-c4ccccc4)ccc(N(c4ccc5c(c4)oc4ccccc45)c4cc(-c5ccccc5)cc5sc6cc(-c7ccccc7)ccc6c45)c23)cc1]

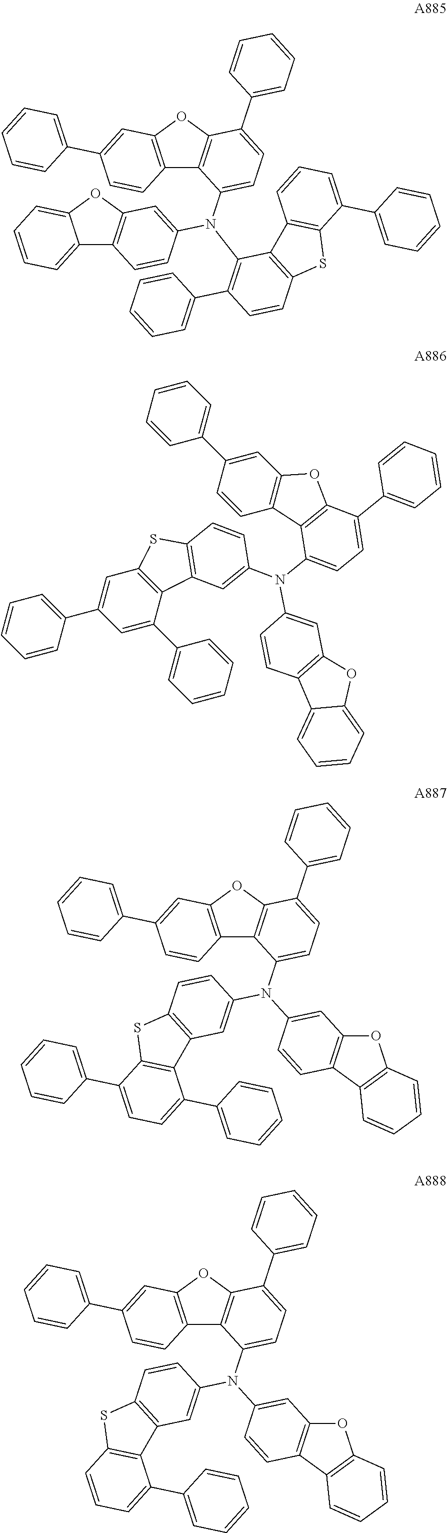

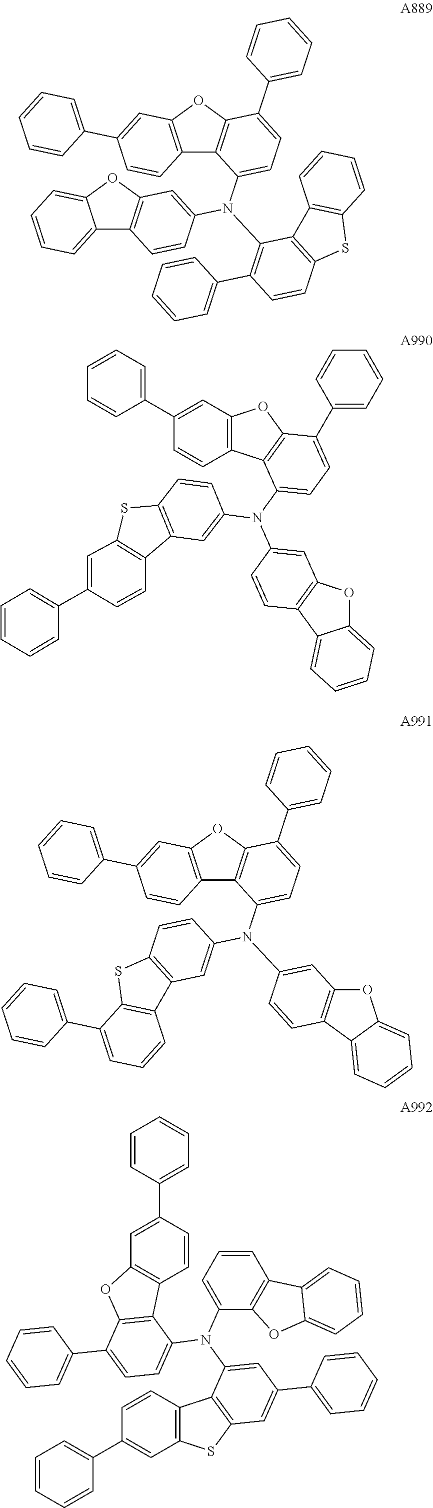 [SMILES: c1ccc(-c2ccc3c(c2)oc2c(-c4ccccc4)ccc(N(c4ccc5c(c4)oc4ccccc45)c4c(-c5ccccc5)ccc5sc6ccccc6c45)c23)cc1 ; c1ccc(-c2ccc3c(c2)oc2c(-c4ccccc4)ccc(N(c4ccc5c(c4)oc4ccccc45)c4ccc5sc6cc(-c7ccccc7)ccc6c5c4)c23)cc1 ; c1ccc(-c2ccc3c(c2)oc2c(-c4ccccc4)ccc(N(c4ccc5c(c4)oc4ccccc45)c4ccc5sc6c(-c7ccccc7)cccc6c5c4)c23)cc1 ; c1ccc(-c2ccc3c(c2)oc2c(-c4ccccc4)ccc(N(c4cccc5c4oc4ccccc45)c4cc(-c5ccccc5)cc5sc6cc(-c7ccccc7)ccc6c45)c23)cc1]

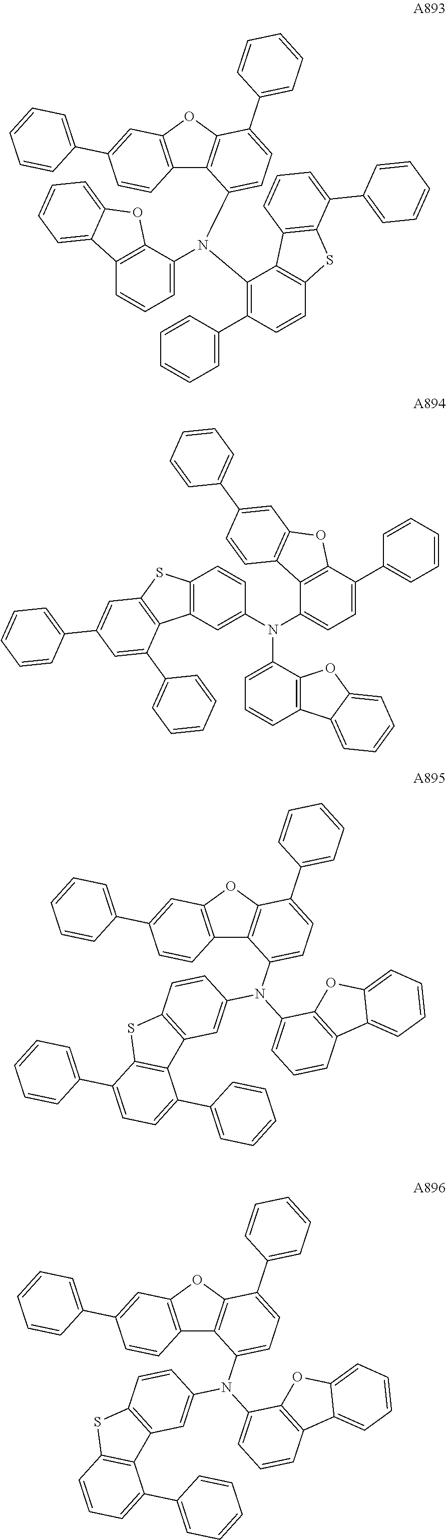 [SMILES: c1ccc(-c2ccc3c(c2)oc2c(-c4ccccc4)ccc(N(c4cccc5c4oc4ccccc45)c4c(-c5ccccc5)ccc5sc6c(-c7ccccc7)cccc6c45)c23)cc1 ; c1ccc(-c2ccc3c(c2)oc2c(-c4ccccc4)ccc(N(c4ccc5sc6cc(-c7ccccc7)cc(-c7ccccc7)c6c5c4)c4cccc5c4oc4ccccc45)c23)cc1 ; c1ccc(-c2ccc3c(c2)oc2c(-c4ccccc4)ccc(N(c4ccc5sc6c(-c7ccccc7)ccc(-c7ccccc7)c6c5c4)c4cccc5c4oc4ccccc45)c23)cc1 ; c1ccc(-c2ccc3c(c2)oc2c(-c4ccccc4)ccc(N(c4ccc5sc6cccc(-c7ccccc7)c6c5c4)c4cccc5c4oc4ccccc45)c23)cc1]

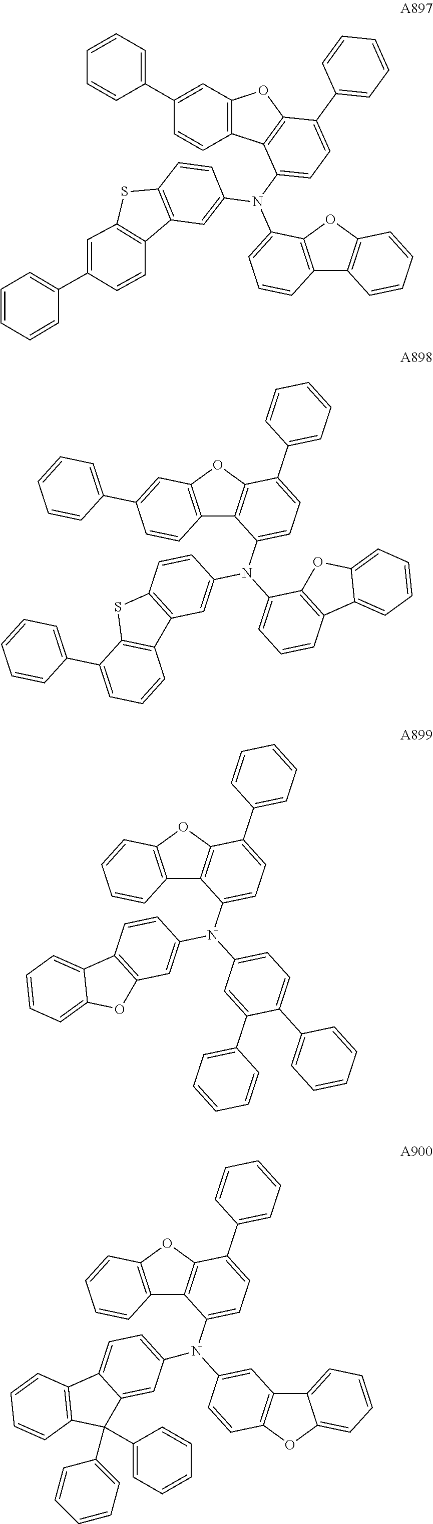 [SMILES: c1ccc(-c2ccc3c(c2)oc2c(-c4ccccc4)ccc(N(c4ccc5sc6cc(-c7ccccc7)ccc6c5c4)c4cccc5c4oc4ccccc45)c23)cc1 ; c1ccc(-c2ccc3c(c2)oc2c(-c4ccccc4)ccc(N(c4ccc5sc6c(-c7ccccc7)cccc6c5c4)c4cccc5c4oc4ccccc45)c23)cc1 ; c1ccc(-c2ccc(N(c3ccc4c(c3)oc3ccccc34)c3ccc(-c4ccccc4)c4oc5ccccc5c34)cc2-c2ccccc2)cc1 ; c1ccc(-c2ccc(N(c3ccc4c(c3)C(c3ccccc3)(c3ccccc3)c3ccccc3-4)c3ccc4oc5ccccc5c4c3)c3c2oc2ccccc23)cc1]

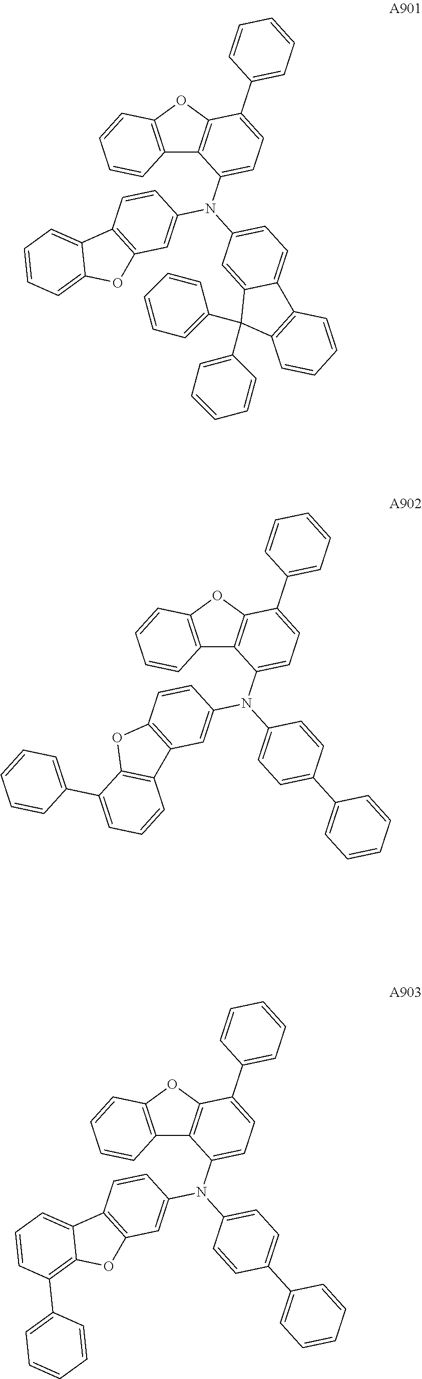 [SMILES: c1ccc(-c2ccc(N(c3ccc4c(c3)C(c3ccccc3)(c3ccccc3)c3ccccc3-4)c3ccc4c(c3)oc3ccccc34)c3c2oc2ccccc23)cc1 ; c1ccc(-c2ccc(N(c3ccc4oc5c(-c6ccccc6)cccc5c4c3)c3ccc(-c4ccccc4)c4oc5ccccc5c34)cc2)cc1 ; c1ccc(-c2ccc(N(c3ccc4c(c3)oc3c(-c5ccccc5)cccc34)c3ccc(-c4ccccc4)c4oc5ccccc5c34)cc2)cc1]

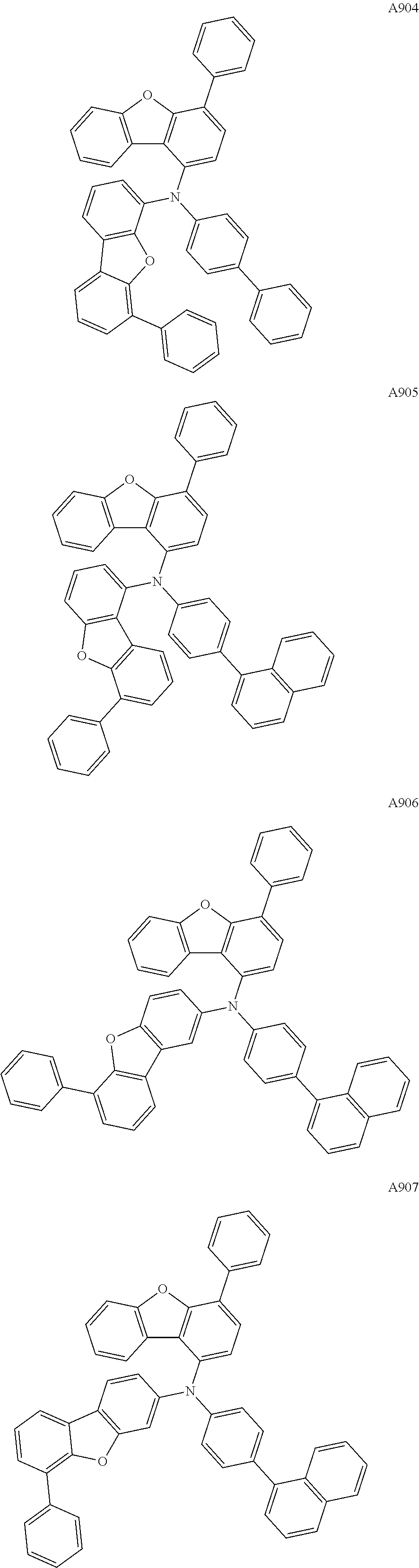

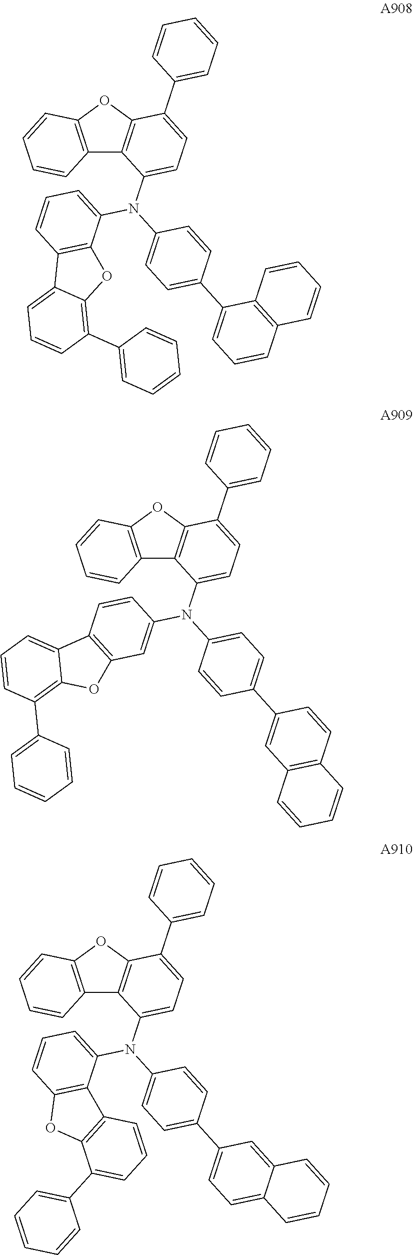 [SMILES: c1ccc(-c2cccc3c2oc2c(N(c4ccc(-c5cccc6ccccc56)cc4)c4ccc(-c5ccccc5)c5oc6ccccc6c45)cccc23)cc1 ; c1ccc(-c2cccc3c2oc2cc(N(c4ccc(-c5ccc6ccccc6c5)cc4)c4ccc(-c5ccccc5)c5oc6ccccc6c45)ccc23)cc1 ; c1ccc(-c2cccc3c2oc2cccc(N(c4ccc(-c5ccc6ccccc6c5)cc4)c4ccc(-c5ccccc5)c5oc6ccccc6c45)c23)cc1]

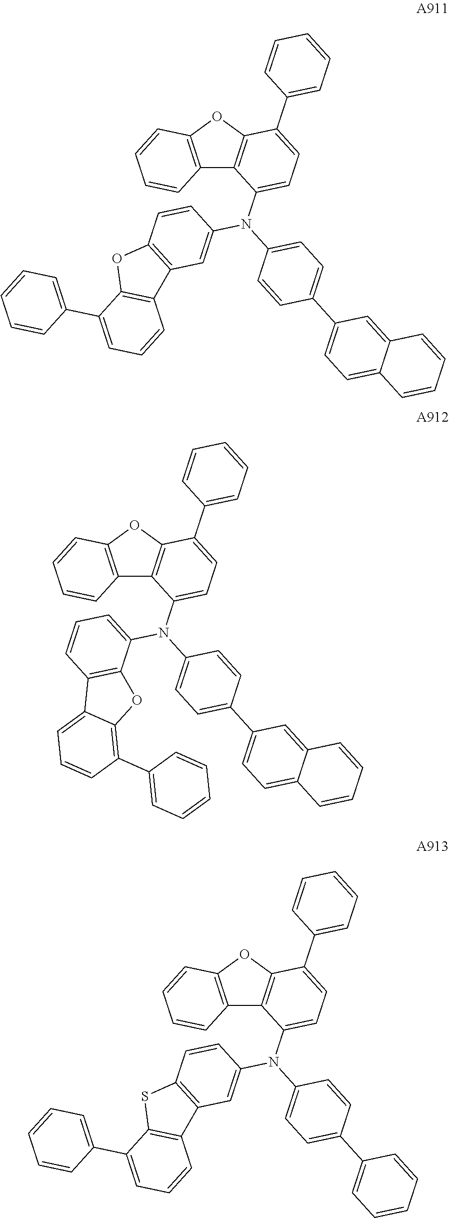 [SMILES: c1ccc(-c2cccc3c2oc2ccc(N(c4ccc(-c5ccc6ccccc6c5)cc4)c4ccc(-c5ccccc5)c5oc6ccccc6c45)cc23)cc1 ; c1ccc(-c2cccc3c2oc2c(N(c4ccc(-c5ccc6ccccc6c5)cc4)c4ccc(-c5ccccc5)c5oc6ccccc6c45)cccc23)cc1 ; c1ccc(-c2ccc(N(c3ccc4sc5c(-c6ccccc6)cccc5c4c3)c3ccc(-c4ccccc4)c4oc5ccccc5c34)cc2)cc1]

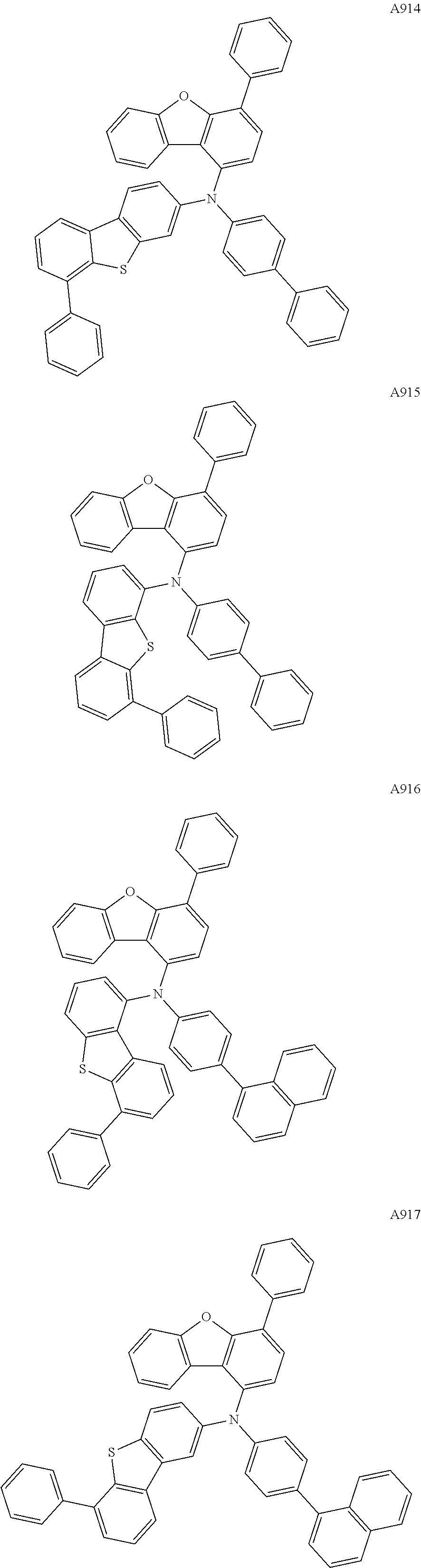 [SMILES: c1ccc(-c2ccc(N(c3ccc4c(c3)sc3c(-c5ccccc5)cccc34)c3ccc(-c4ccccc4)c4oc5ccccc5c34)cc2)cc1 ; c1ccc(-c2ccc(N(c3cccc4c3sc3c(-c5ccccc5)cccc34)c3ccc(-c4ccccc4)c4oc5ccccc5c34)cc2)cc1 ; c1ccc(-c2ccc(N(c3ccc(-c4cccc5ccccc45)cc3)c3cccc4sc5c(-c6ccccc6)cccc5c34)c3c2oc2ccccc23)cc1 ; c1ccc(-c2ccc(N(c3ccc(-c4cccc5ccccc45)cc3)c3ccc4sc5c(-c6ccccc6)cccc5c4c3)c3c2oc2ccccc23)cc1]

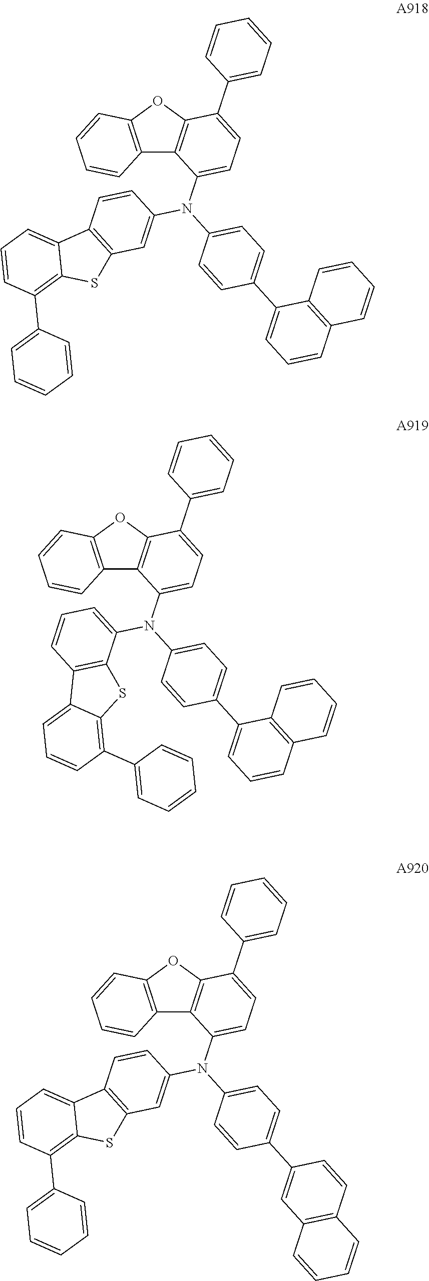 [SMILES: c1ccc(-c2ccc(N(c3ccc(-c4cccc5ccccc45)cc3)c3ccc4c(c3)sc3c(-c5ccccc5)cccc34)c3c2oc2ccccc23)cc1 ; c1ccc(-c2ccc(N(c3ccc(-c4cccc5ccccc45)cc3)c3cccc4c3sc3c(-c5ccccc5)cccc34)c3c2oc2ccccc23)cc1 ; c1ccc(-c2ccc(N(c3ccc(-c4ccc5ccccc5c4)cc3)c3ccc4c(c3)sc3c(-c5ccccc5)cccc34)c3c2oc2ccccc23)cc1]

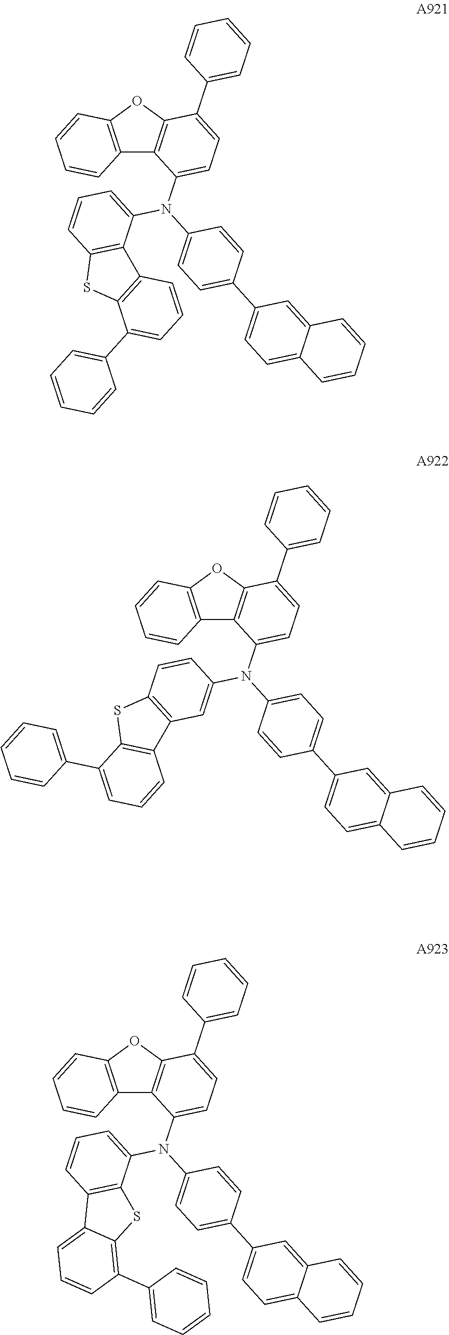 [SMILES: c1ccc(-c2ccc(N(c3ccc(-c4ccc5ccccc5c4)cc3)c3cccc4sc5c(-c6ccccc6)cccc5c34)c3c2oc2ccccc23)cc1 ; c1ccc(-c2ccc(N(c3ccc(-c4ccc5ccccc5c4)cc3)c3ccc4sc5c(-c6ccccc6)cccc5c4c3)c3c2oc2ccccc23)cc1 ; c1ccc(-c2ccc(N(c3ccc(-c4ccc5ccccc5c4)cc3)c3cccc4c3sc3c(-c5ccccc5)cccc34)c3c2oc2ccccc23)cc1]

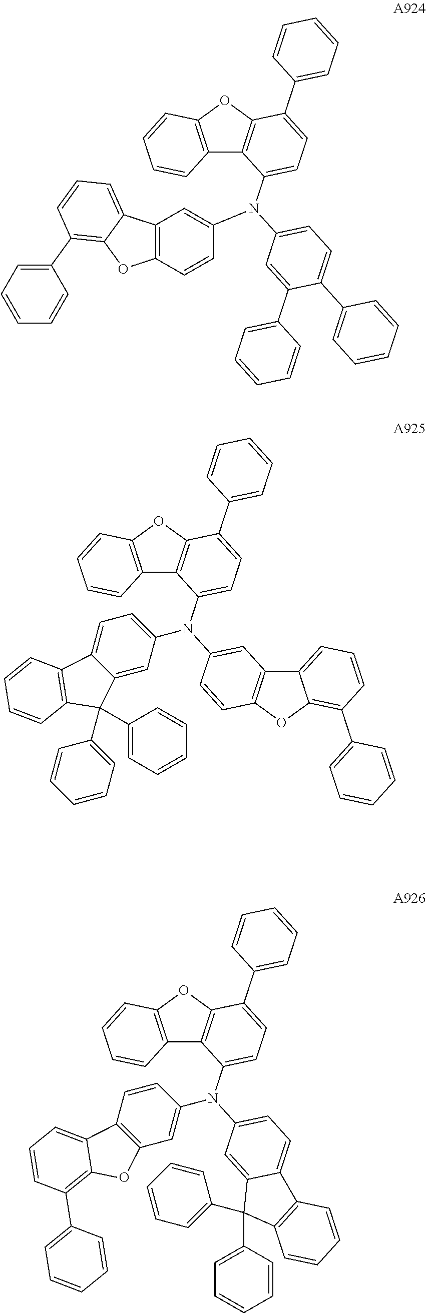 [SMILES: c1ccc(-c2ccc(N(c3ccc4oc5c(-c6ccccc6)cccc5c4c3)c3ccc(-c4ccccc4)c4oc5ccccc5c34)cc2-c2ccccc2)cc1 ; c1ccc(-c2cccc3c2oc2ccc(N(c4ccc5c(c4)C(c4ccccc4)(c4ccccc4)c4ccccc4-5)c4ccc(-c5ccccc5)c5oc6ccccc6c45)cc23)cc1 ; c1ccc(-c2cccc3c2oc2cc(N(c4ccc5c(c4)C(c4ccccc4)(c4ccccc4)c4ccccc4-5)c4ccc(-c5ccccc5)c5oc6ccccc6c45)ccc23)cc1]

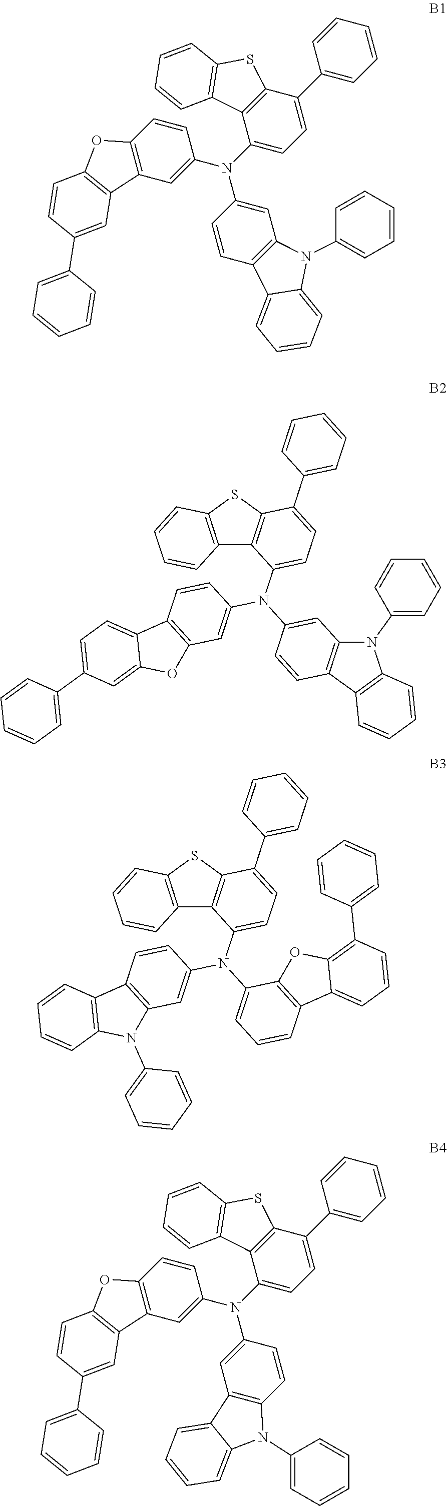 [SMILES: c1ccc(-c2ccc3oc4ccc(N(c5ccc6c7ccccc7n(-c7ccccc7)c6c5)c5ccc(-c6ccccc6)c6sc7ccccc7c56)cc4c3c2)cc1 ; c1ccc(-c2ccc3c(c2)oc2cc(N(c4ccc5c6ccccc6n(-c6ccccc6)c5c4)c4ccc(-c5ccccc5)c5sc6ccccc6c45)ccc23)cc1 ; c1ccc(-c2cccc3c2oc2c(N(c4ccc5c6ccccc6n(-c6ccccc6)c5c4)c4ccc(-c5ccccc5)c5sc6ccccc6c45)cccc23)cc1 ; c1ccc(-c2ccc3oc4ccc(N(c5ccc6c(c5)c5ccccc5n6-c5ccccc5)c5ccc(-c6ccccc6)c6sc7ccccc7c56)cc4c3c2)cc1]

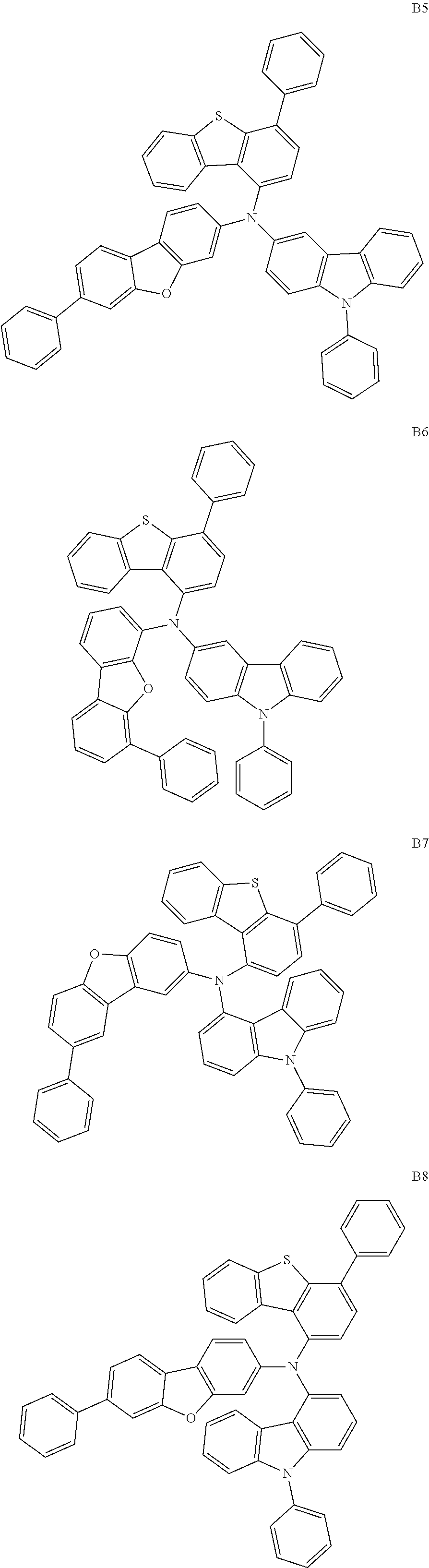 [SMILES: c1ccc(-c2ccc3c(c2)oc2cc(N(c4ccc5c(c4)c4ccccc4n5-c4ccccc4)c4ccc(-c5ccccc5)c5sc6ccccc6c45)ccc23)cc1 ; c1ccc(-c2cccc3c2oc2c(N(c4ccc5c(c4)c4ccccc4n5-c4ccccc4)c4ccc(-c5ccccc5)c5sc6ccccc6c45)cccc23)cc1 ; c1ccc(-c2ccc3oc4ccc(N(c5ccc(-c6ccccc6)c6sc7ccccc7c56)c5cccc6c5c5ccccc5n6-c5ccccc5)cc4c3c2)cc1 ; c1ccc(-c2ccc3c(c2)oc2cc(N(c4ccc(-c5ccccc5)c5sc6ccccc6c45)c4cccc5c4c4ccccc4n5-c4ccccc4)ccc23)cc1]

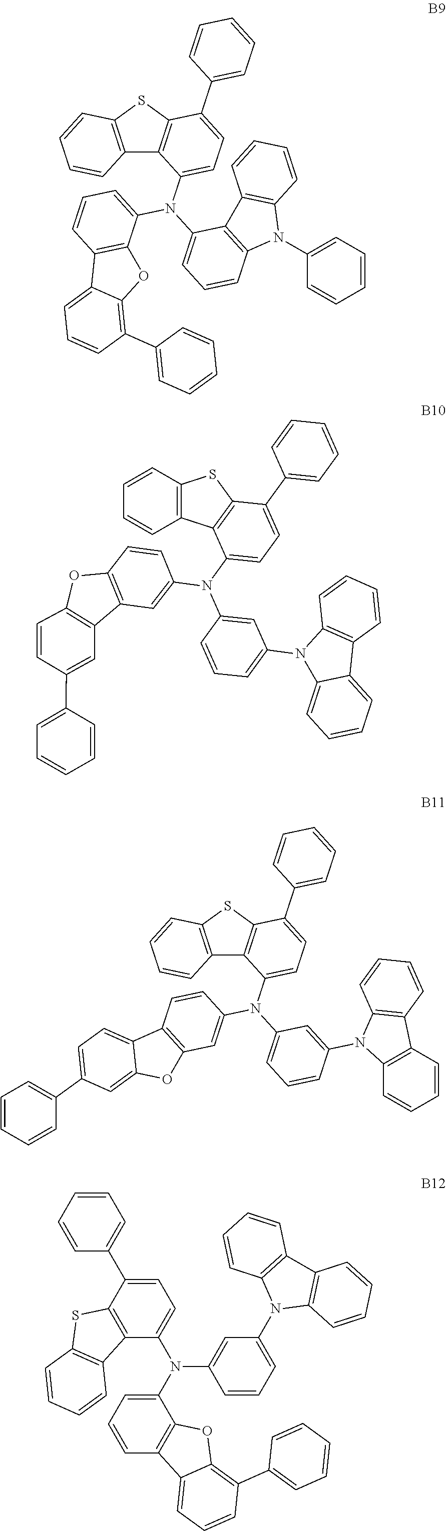 [SMILES: c1ccc(-c2cccc3c2oc2c(N(c4ccc(-c5ccccc5)c5sc6ccccc6c45)c4cccc5c4c4ccccc4n5-c4ccccc4)cccc23)cc1 ; c1ccc(-c2ccc3oc4ccc(N(c5cccc(-n6c7ccccc7c7ccccc76)c5)c5ccc(-c6ccccc6)c6sc7ccccc7c56)cc4c3c2)cc1 ; c1ccc(-c2ccc3c(c2)oc2cc(N(c4cccc(-n5c6ccccc6c6ccccc65)c4)c4ccc(-c5ccccc5)c5sc6ccccc6c45)ccc23)cc1 ; c1ccc(-c2cccc3c2oc2c(N(c4cccc(-n5c6ccccc6c6ccccc65)c4)c4ccc(-c5ccccc5)c5sc6ccccc6c45)cccc23)cc1]

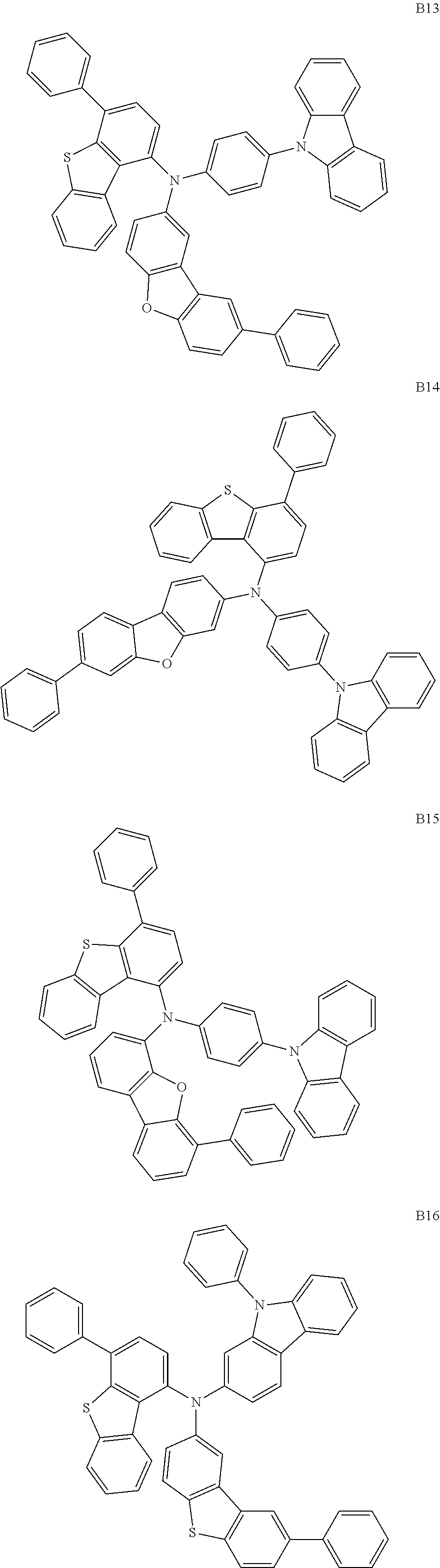

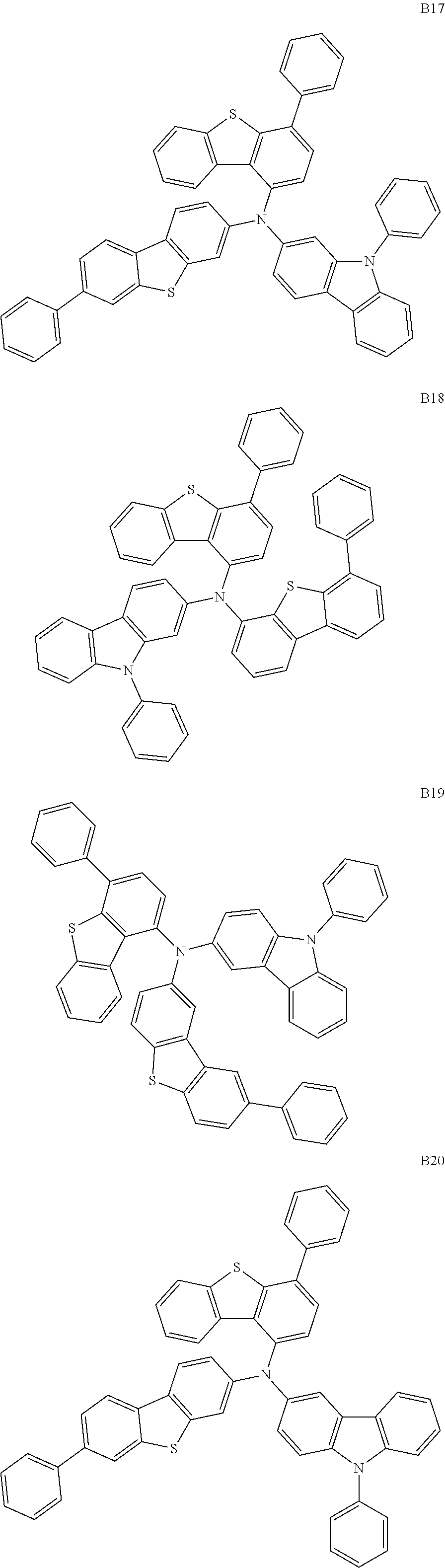

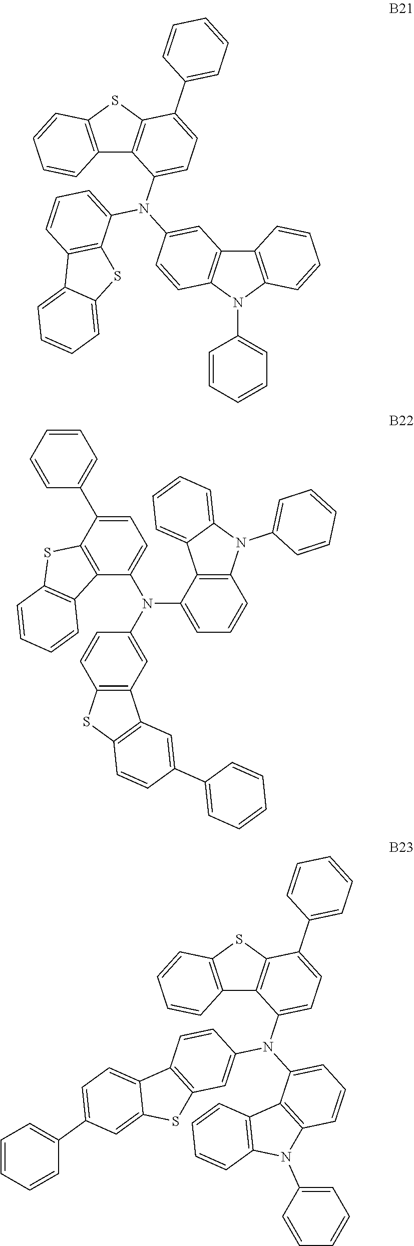 [SMILES: c1ccc(-c2ccc(N(c3ccc4c(c3)c3ccccc3n4-c3ccccc3)c3cccc4c3sc3ccccc34)c3c2sc2ccccc23)cc1 ; c1ccc(-c2ccc3sc4ccc(N(c5ccc(-c6ccccc6)c6sc7ccccc7c56)c5cccc6c5c5ccccc5n6-c5ccccc5)cc4c3c2)cc1 ; c1ccc(-c2ccc3c(c2)sc2cc(N(c4ccc(-c5ccccc5)c5sc6ccccc6c45)c4cccc5c4c4ccccc4n5-c4ccccc4)ccc23)cc1]

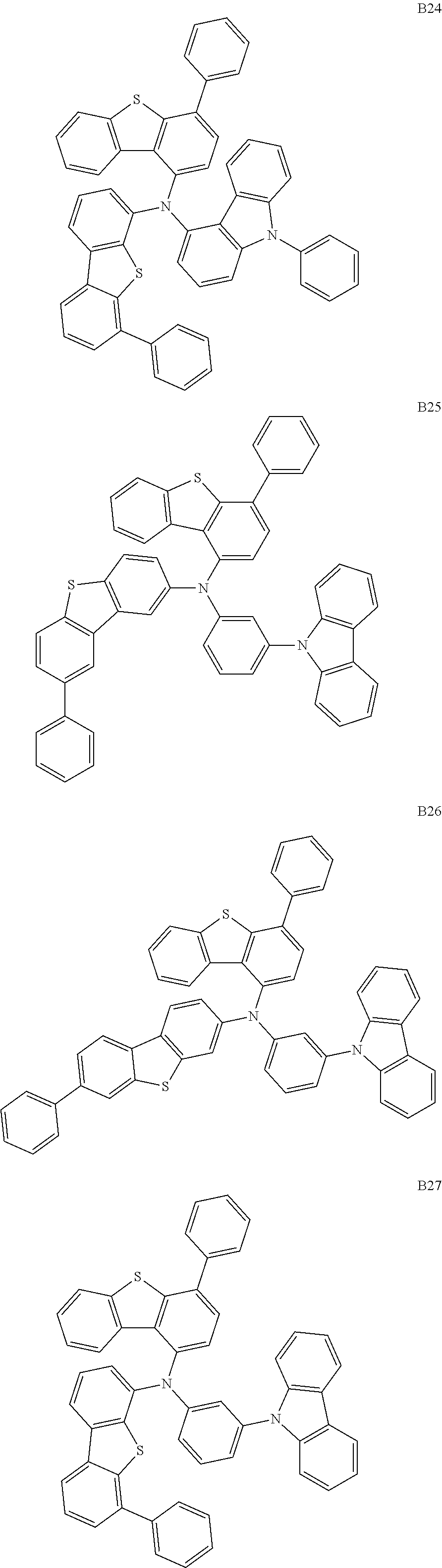 [SMILES: c1ccc(-c2cccc3c2sc2c(N(c4ccc(-c5ccccc5)c5sc6ccccc6c45)c4cccc5c4c4ccccc4n5-c4ccccc4)cccc23)cc1 ; c1ccc(-c2ccc3sc4ccc(N(c5cccc(-n6c7ccccc7c7ccccc76)c5)c5ccc(-c6ccccc6)c6sc7ccccc7c56)cc4c3c2)cc1 ; c1ccc(-c2ccc3c(c2)sc2cc(N(c4cccc(-n5c6ccccc6c6ccccc65)c4)c4ccc(-c5ccccc5)c5sc6ccccc6c45)ccc23)cc1 ; c1ccc(-c2cccc3c2sc2c(N(c4cccc(-n5c6ccccc6c6ccccc65)c4)c4ccc(-c5ccccc5)c5sc6ccccc6c45)cccc23)cc1]

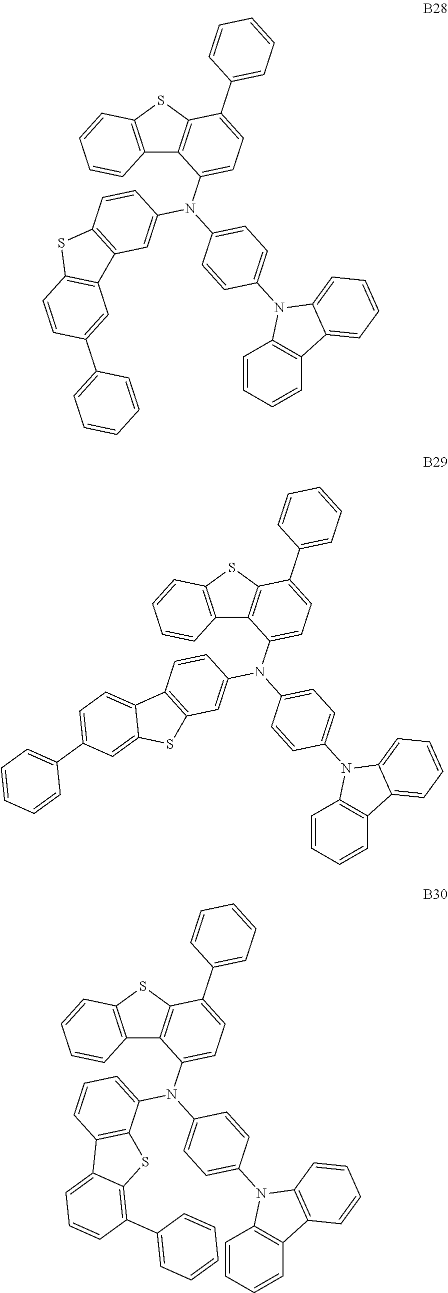 [SMILES: c1ccc(-c2ccc3sc4ccc(N(c5ccc(-n6c7ccccc7c7ccccc76)cc5)c5ccc(-c6ccccc6)c6sc7ccccc7c56)cc4c3c2)cc1 ; c1ccc(-c2ccc3c(c2)sc2cc(N(c4ccc(-n5c6ccccc6c6ccccc65)cc4)c4ccc(-c5ccccc5)c5sc6ccccc6c45)ccc23)cc1 ; c1ccc(-c2cccc3c2sc2c(N(c4ccc(-n5c6ccccc6c6ccccc65)cc4)c4ccc(-c5ccccc5)c5sc6ccccc6c45)cccc23)cc1]

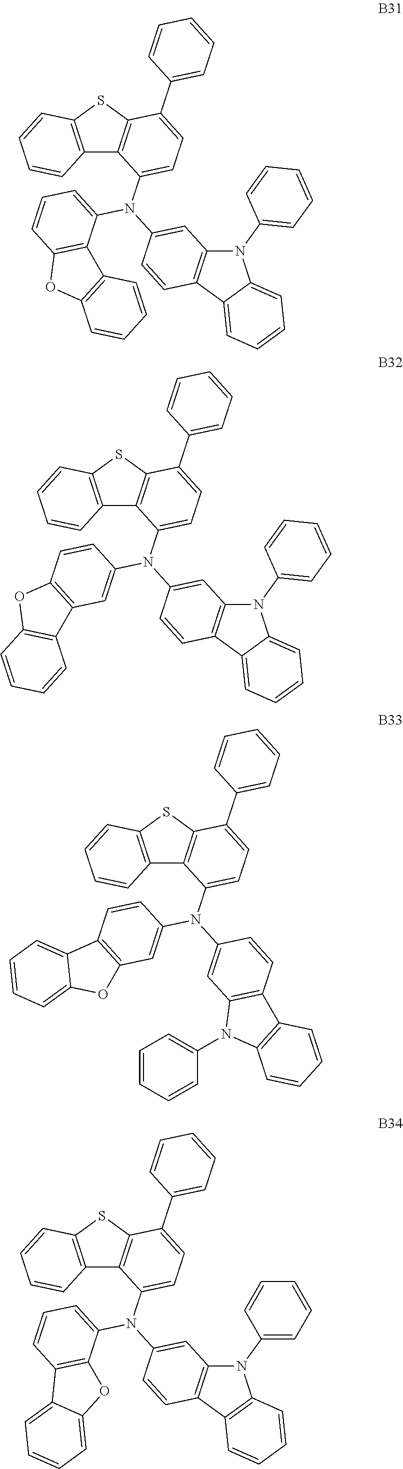

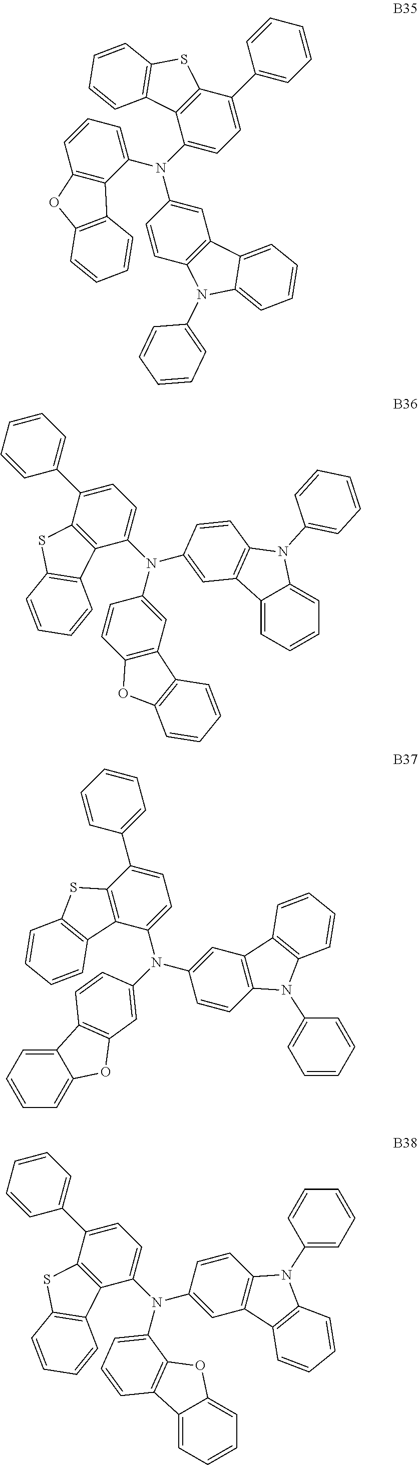

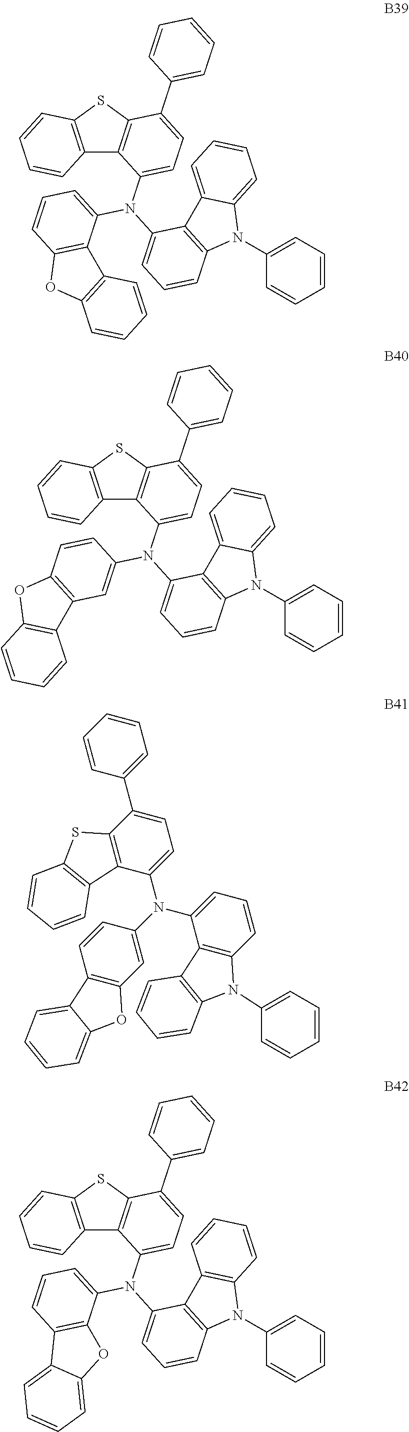 [SMILES: c1ccc(-c2ccc(N(c3cccc4oc5ccccc5c34)c3cccc4c3c3ccccc3n4-c3ccccc3)c3c2sc2ccccc23)cc1 ; c1ccc(-c2ccc(N(c3ccc4oc5ccccc5c4c3)c3cccc4c3c3ccccc3n4-c3ccccc3)c3c2sc2ccccc23)cc1 ; c1ccc(-c2ccc(N(c3ccc4c(c3)oc3ccccc34)c3cccc4c3c3ccccc3n4-c3ccccc3)c3c2sc2ccccc23)cc1 ; c1ccc(-c2ccc(N(c3cccc4c3oc3ccccc34)c3cccc4c3c3ccccc3n4-c3ccccc3)c3c2sc2ccccc23)cc1]

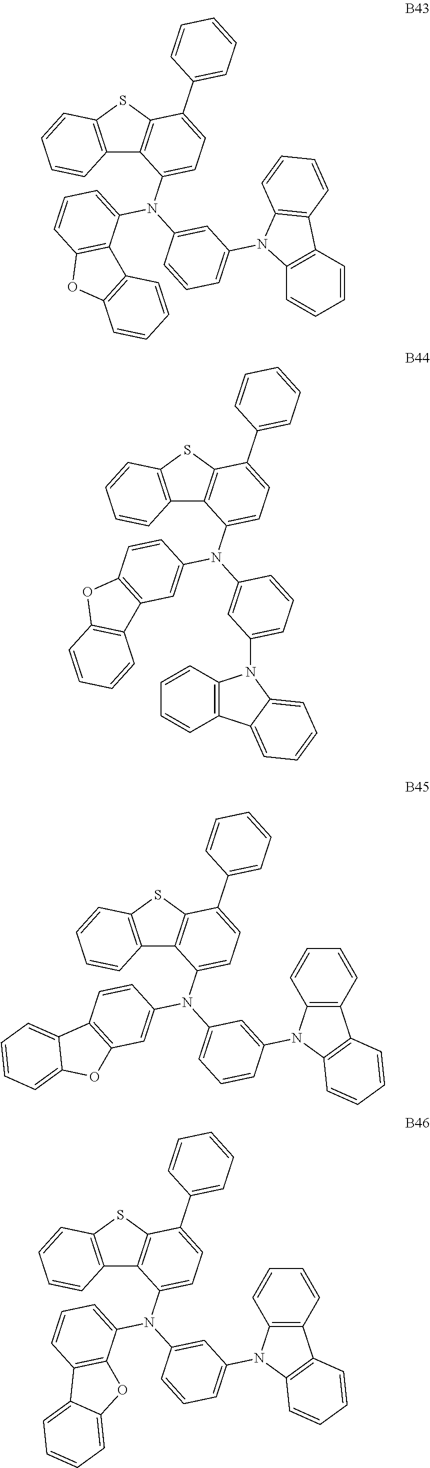 [SMILES: c1ccc(-c2ccc(N(c3cccc(-n4c5ccccc5c5ccccc54)c3)c3cccc4oc5ccccc5c34)c3c2sc2ccccc23)cc1 ; c1ccc(-c2ccc(N(c3cccc(-n4c5ccccc5c5ccccc54)c3)c3ccc4oc5ccccc5c4c3)c3c2sc2ccccc23)cc1 ; c1ccc(-c2ccc(N(c3cccc(-n4c5ccccc5c5ccccc54)c3)c3ccc4c(c3)oc3ccccc34)c3c2sc2ccccc23)cc1 ; c1ccc(-c2ccc(N(c3cccc(-n4c5ccccc5c5ccccc54)c3)c3cccc4c3oc3ccccc34)c3c2sc2ccccc23)cc1]

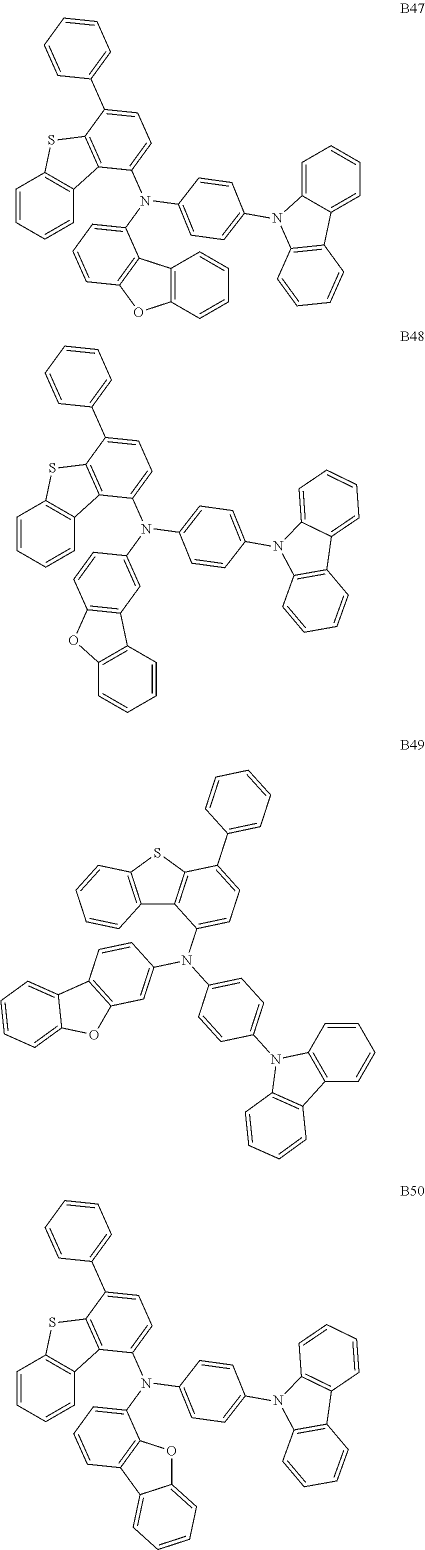 [SMILES: c1ccc(-c2ccc(N(c3ccc(-n4c5ccccc5c5ccccc54)cc3)c3cccc4oc5ccccc5c34)c3c2sc2ccccc23)cc1 ; c1ccc(-c2ccc(N(c3ccc(-n4c5ccccc5c5ccccc54)cc3)c3ccc4oc5ccccc5c4c3)c3c2sc2ccccc23)cc1 ; c1ccc(-c2ccc(N(c3ccc(-n4c5ccccc5c5ccccc54)cc3)c3ccc4c(c3)oc3ccccc34)c3c2sc2ccccc23)cc1 ; c1ccc(-c2ccc(N(c3ccc(-n4c5ccccc5c5ccccc54)cc3)c3cccc4c3oc3ccccc34)c3c2sc2ccccc23)cc1]

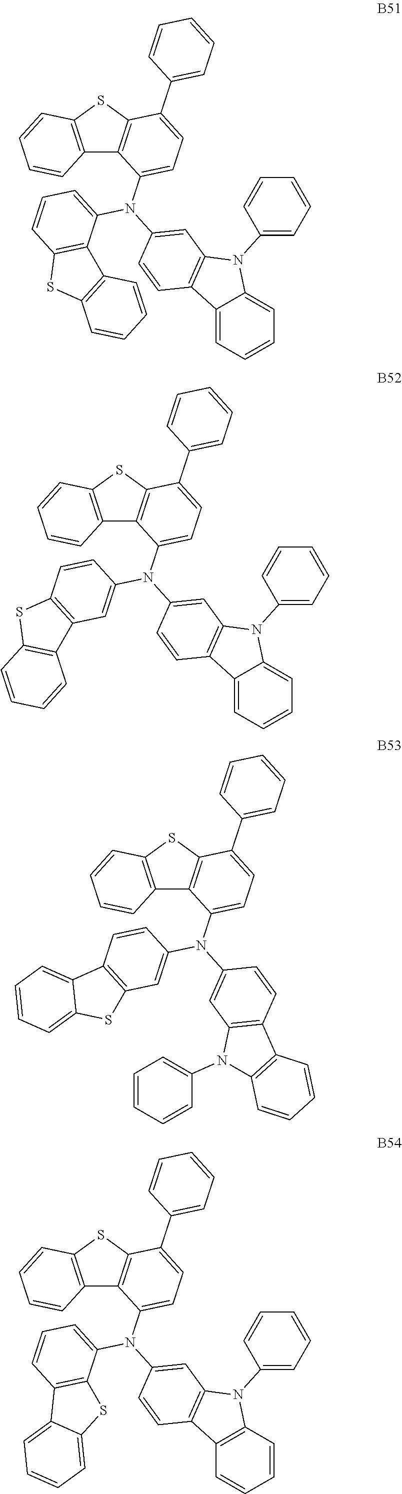

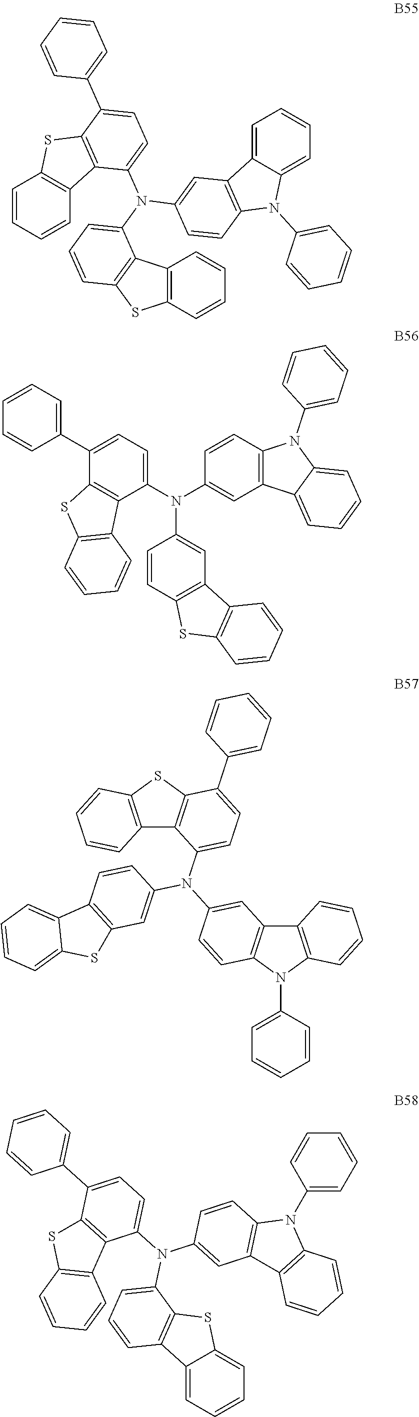

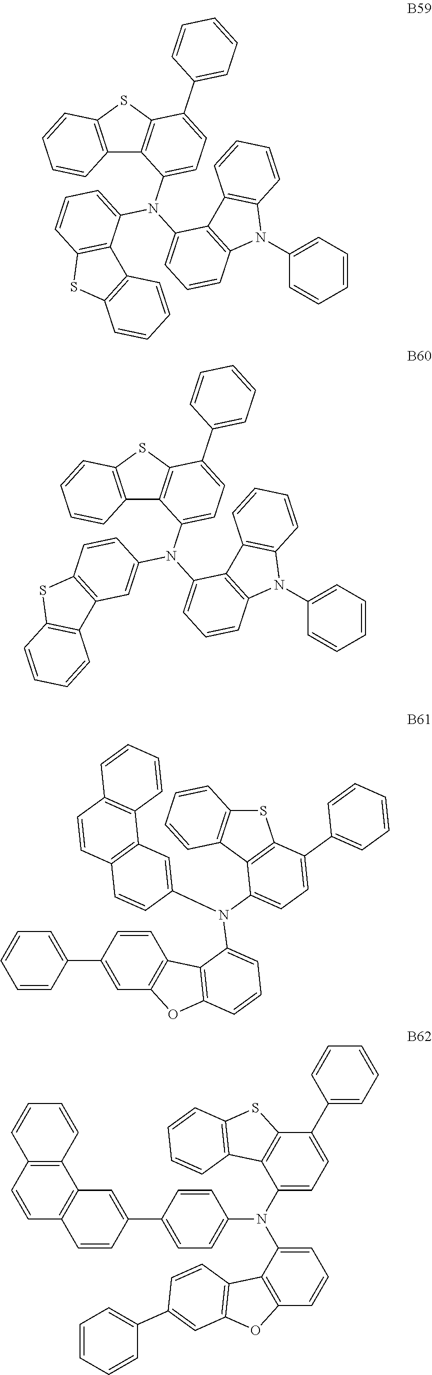 [SMILES: c1ccc(-c2ccc(N(c3cccc4sc5ccccc5c34)c3cccc4c3c3ccccc3n4-c3ccccc3)c3c2sc2ccccc23)cc1 ; c1ccc(-c2ccc(N(c3ccc4sc5ccccc5c4c3)c3cccc4c3c3ccccc3n4-c3ccccc3)c3c2sc2ccccc23)cc1 ; c1ccc(-c2ccc3c(c2)oc2cccc(N(c4ccc5ccc6ccccc6c5c4)c4ccc(-c5ccccc5)c5sc6ccccc6c45)c23)cc1 ; c1ccc(-c2ccc3c(c2)oc2cccc(N(c4ccc(-c5ccc6ccc7ccccc7c6c5)cc4)c4ccc(-c5ccccc5)c5sc6ccccc6c45)c23)cc1]

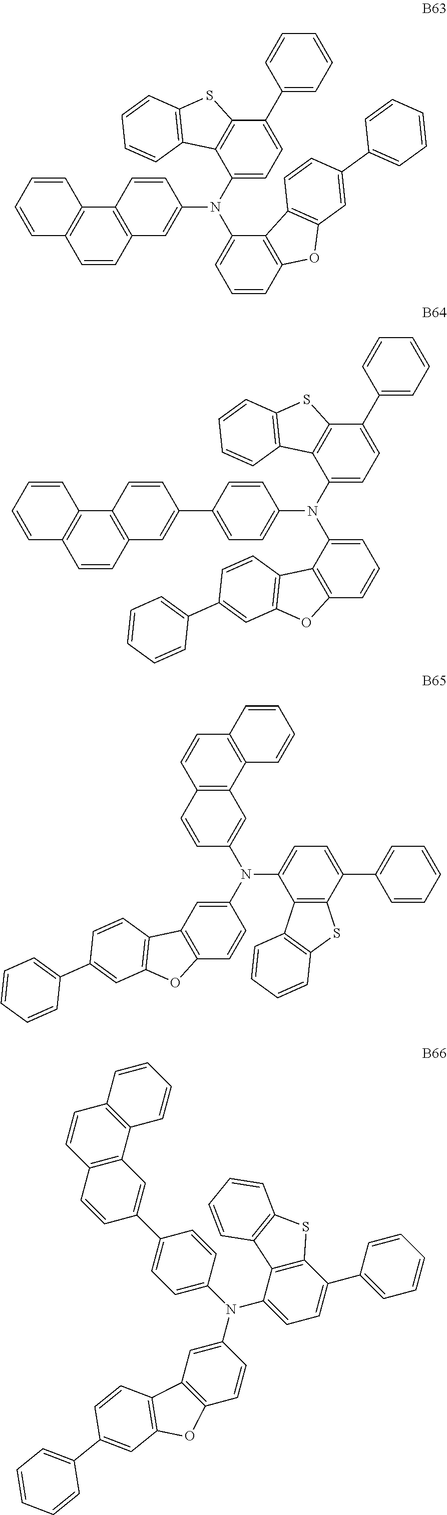 [SMILES: c1ccc(-c2ccc3c(c2)oc2cccc(N(c4ccc5c(ccc6ccccc65)c4)c4ccc(-c5ccccc5)c5sc6ccccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5sc6ccccc6c45)c23)cc1 ; c1ccc(-c2ccc3c(c2)oc2ccc(N(c4ccc5ccc6ccccc6c5c4)c4ccc(-c5ccccc5)c5sc6ccccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5sc6ccccc6c45)cc23)cc1]

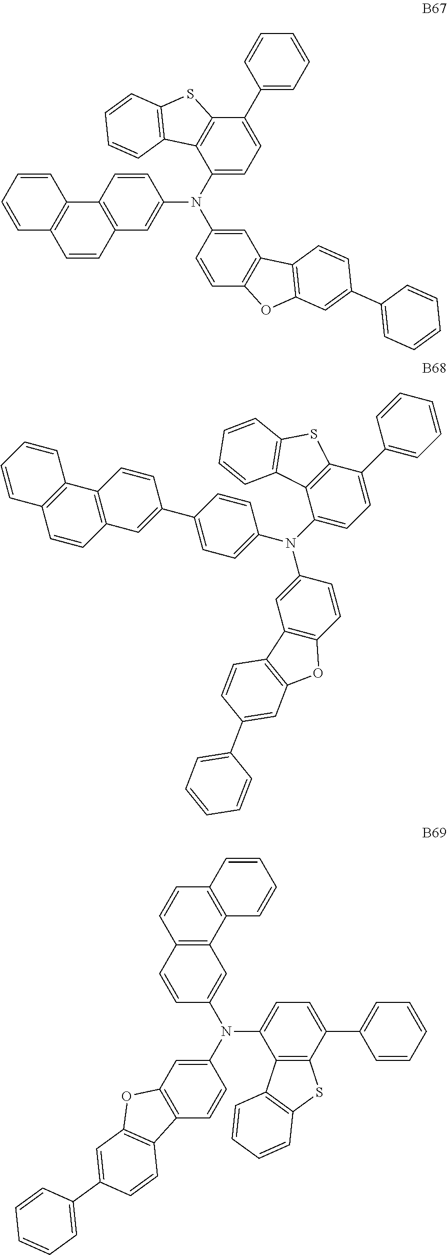

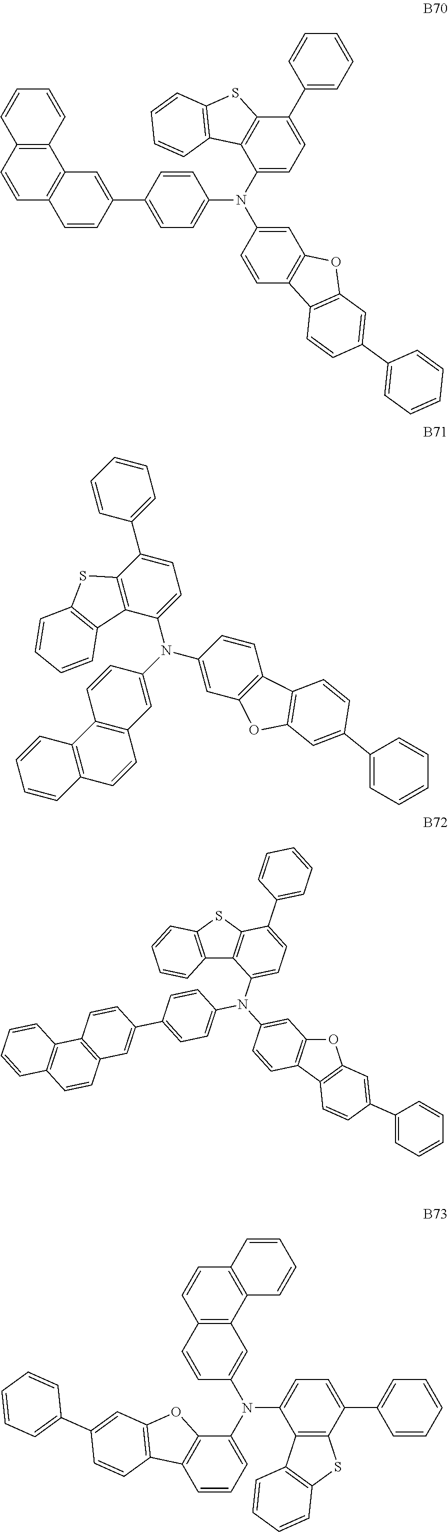 [SMILES: c1ccc(-c2ccc3c(c2)oc2cc(N(c4ccc(-c5ccc6ccc7ccccc7c6c5)cc4)c4ccc(-c5ccccc5)c5sc6ccccc6c45)ccc23)cc1 ; c1ccc(-c2ccc3c(c2)oc2cc(N(c4ccc5c(ccc6ccccc65)c4)c4ccc(-c5ccccc5)c5sc6ccccc6c45)ccc23)cc1 ; c1ccc(-c2ccc3c(c2)oc2cc(N(c4ccc(-c5ccc6c(ccc7ccccc76)c5)cc4)c4ccc(-c5ccccc5)c5sc6ccccc6c45)ccc23)cc1 ; c1ccc(-c2ccc3c(c2)oc2c(N(c4ccc5ccc6ccccc6c5c4)c4ccc(-c5ccccc5)c5sc6ccccc6c45)cccc23)cc1]

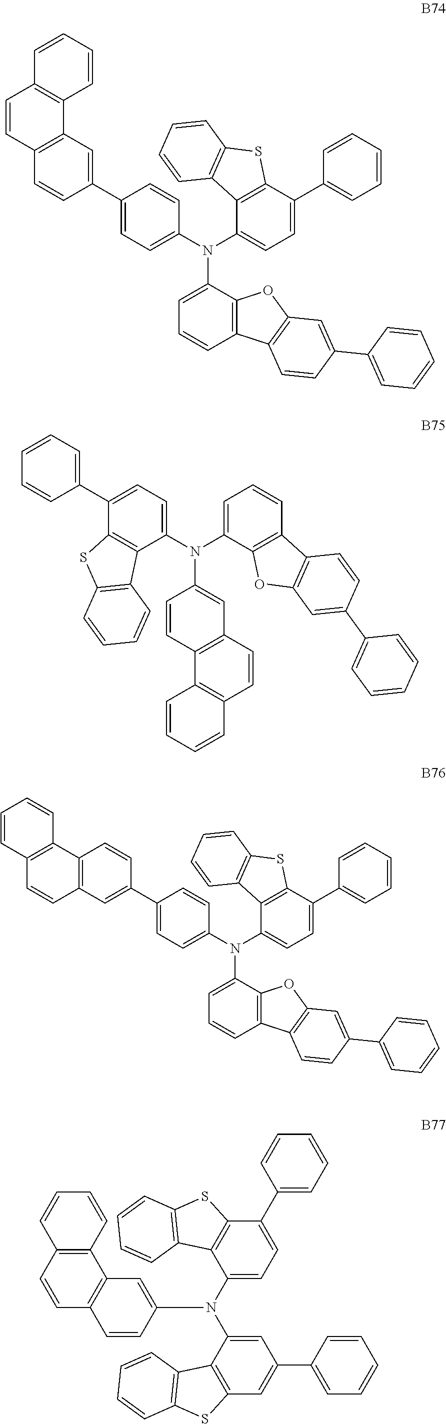 [SMILES: c1ccc(-c2ccc3c(c2)oc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)oc2c(N(c4ccc5c(ccc6ccccc65)c4)c4ccc(-c5ccccc5)c5sc6ccccc6c45)cccc23)cc1 ; c1ccc(-c2ccc3c(c2)oc2c(N(c4ccc(-c5ccc6c(ccc7ccccc76)c5)cc4)c4ccc(-c5ccccc5)c5sc6ccccc6c45)cccc23)cc1 ; c1ccc(-c2cc(N(c3ccc4ccc5ccccc5c4c3)c3ccc(-c4ccccc4)c4sc5ccccc5c34)c3c(c2)sc2ccccc23)cc1]

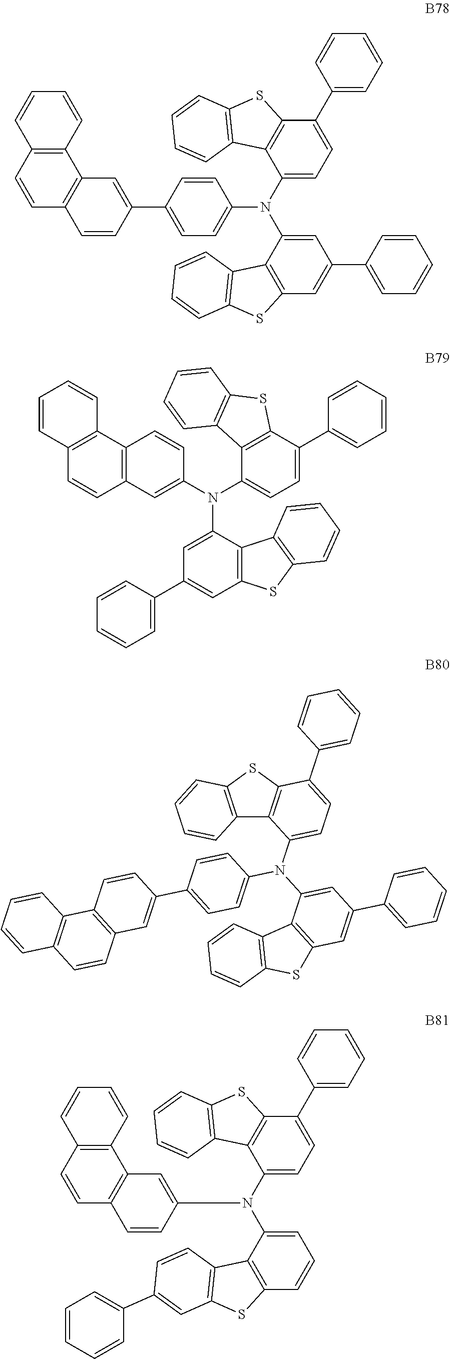 [SMILES: c1ccc(-c2cc(N(c3ccc(-c4ccc5ccc6ccccc6c5c4)cc3)c3ccc(-c4ccccc4)c4sc5ccccc5c34)c3c(c2)sc2ccccc23)cc1 ; c1ccc(-c2cc(N(c3ccc4c(ccc5ccccc54)c3)c3ccc(-c4ccccc4)c4sc5ccccc5c34)c3c(c2)sc2ccccc23)cc1 ; c1ccc(-c2cc(N(c3ccc(-c4ccc5c(ccc6ccccc65)c4)cc3)c3ccc(-c4ccccc4)c4sc5ccccc5c34)c3c(c2)sc2ccccc23)cc1 ; c1ccc(-c2ccc3c(c2)sc2cccc(N(c4ccc5ccc6ccccc6c5c4)c4ccc(-c5ccccc5)c5sc6ccccc6c45)c23)cc1]

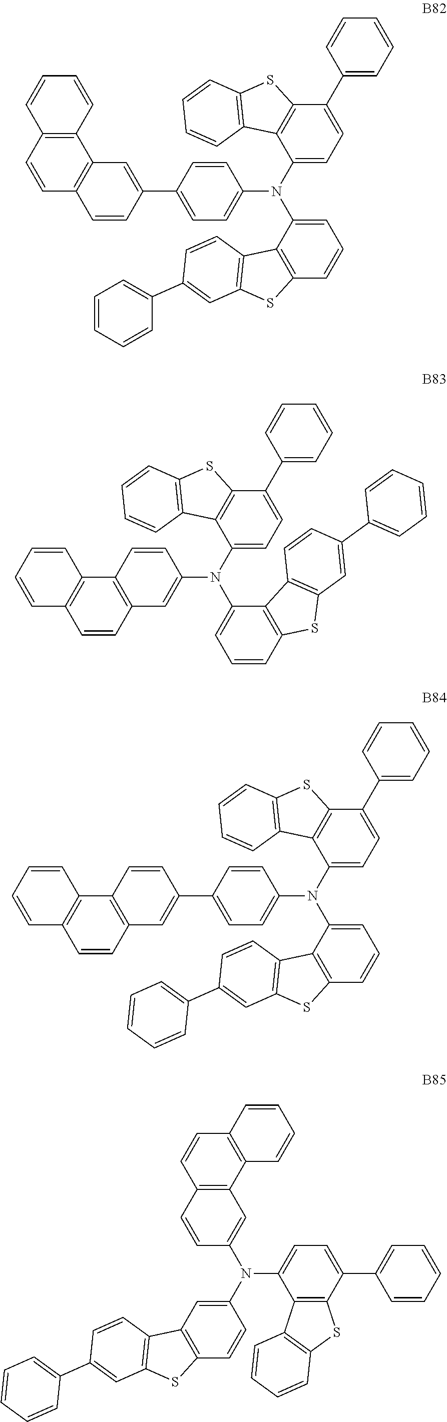 [SMILES: c1ccc(-c2ccc3c(c2)sc2cccc(N(c4ccc(-c5ccc6ccc7ccccc7c6c5)cc4)c4ccc(-c5ccccc5)c5sc6ccccc6c45)c23)cc1 ; c1ccc(-c2ccc3c(c2)sc2cccc(N(c4ccc5c(ccc6ccccc65)c4)c4ccc(-c5ccccc5)c5sc6ccccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5sc6ccccc6c45)c23)cc1 ; c1ccc(-c2ccc3c(c2)sc2ccc(N(c4ccc5ccc6ccccc6c5c4)c4ccc(-c5ccccc5)c5sc6ccccc6c45)cc23)cc1]

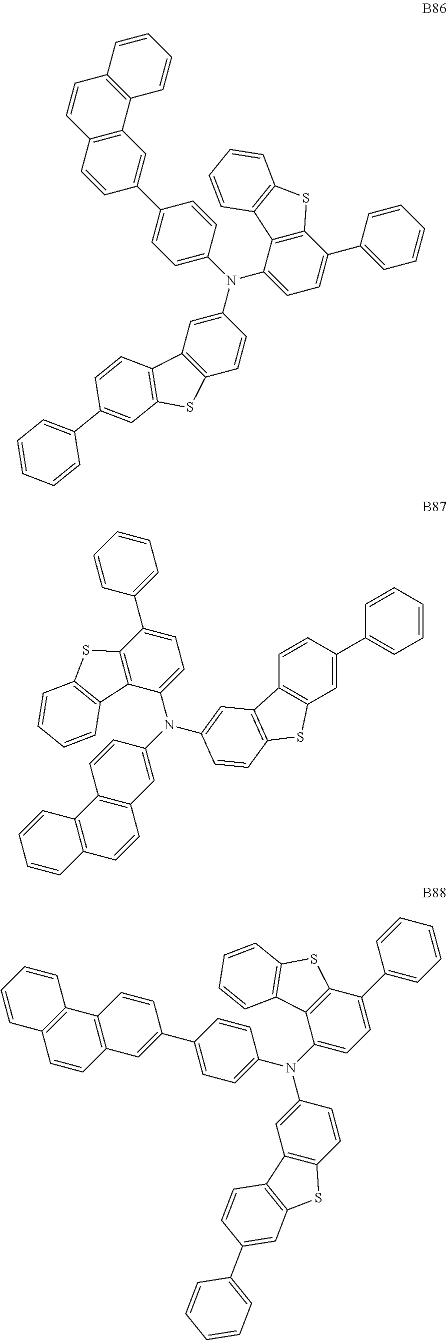 [SMILES: c1ccc(-c2ccc3c(c2)sc2ccc(N(c4ccc(-c5ccc6ccc7ccccc7c6c5)cc4)c4ccc(-c5ccccc5)c5sc6ccccc6c45)cc23)cc1 ; c1ccc(-c2ccc3c(c2)sc2ccc(N(c4ccc5c(ccc6ccccc65)c4)c4ccc(-c5ccccc5)c5sc6ccccc6c45)cc23)cc1 ; c1ccc(-c2ccc3c(c2)sc2ccc(N(c4ccc(-c5ccc6c(ccc7ccccc76)c5)cc4)c4ccc(-c5ccccc5)c5sc6ccccc6c45)cc23)cc1]

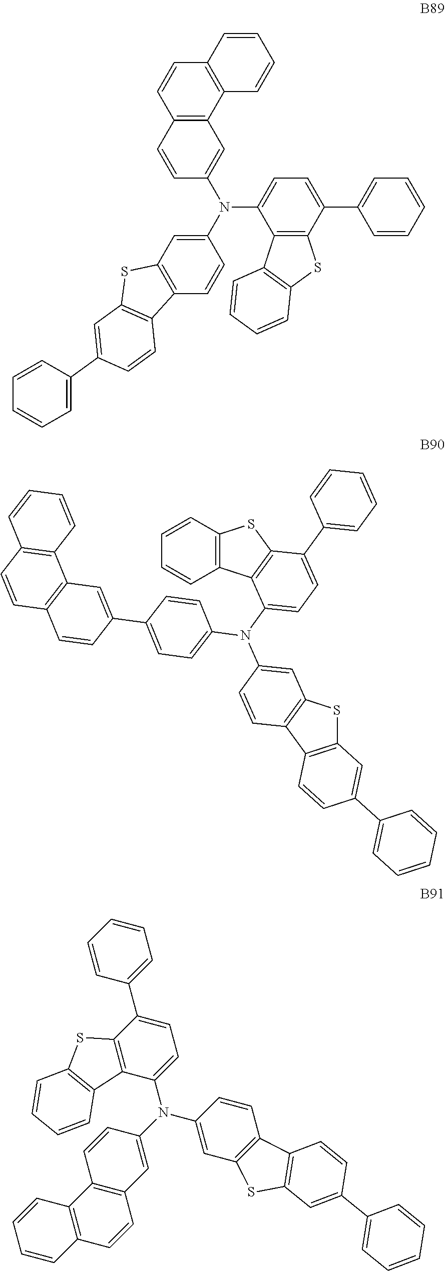 [SMILES: c1ccc(-c2ccc3c(c2)sc2cc(N(c4ccc5ccc6ccccc6c5c4)c4ccc(-c5ccccc5)c5sc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5sc6ccccc6c45)ccc23)cc1 ; c1ccc(-c2ccc3c(c2)sc2cc(N(c4ccc5c(ccc6ccccc65)c4)c4ccc(-c5ccccc5)c5sc6ccccc6c45)ccc23)cc1]

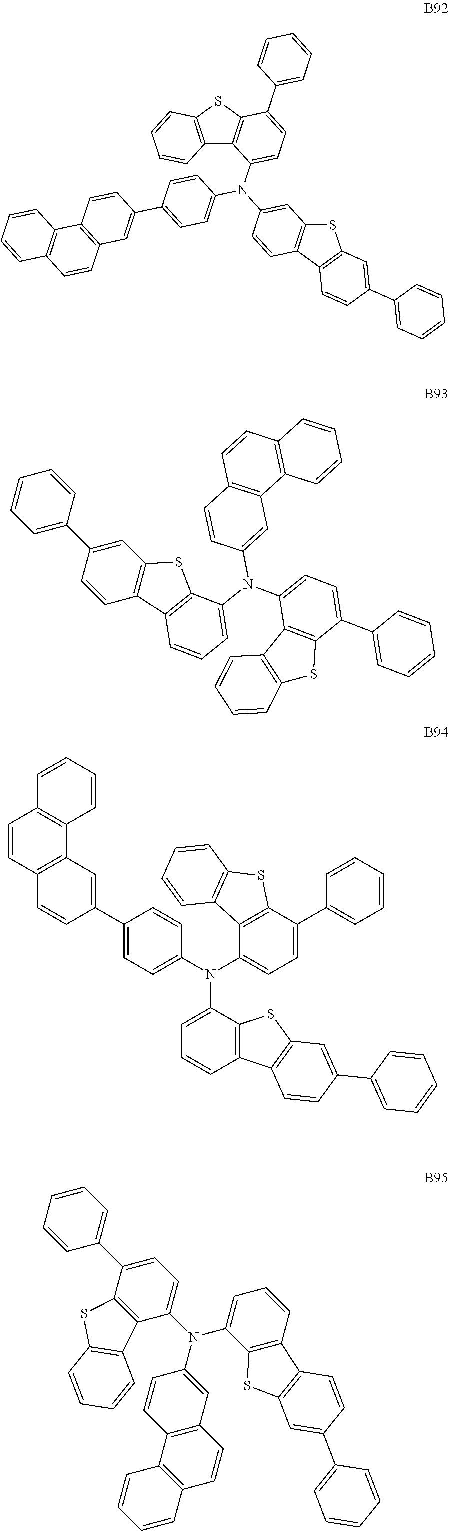 [SMILES: c1ccc(-c2ccc3c(c2)sc2cc(N(c4ccc(-c5ccc6c(ccc7ccccc76)c5)cc4)c4ccc(-c5ccccc5)c5sc6ccccc6c45)ccc23)cc1 ; c1ccc(-c2ccc3c(c2)sc2c(N(c4ccc5ccc6ccccc6c5c4)c4ccc(-c5ccccc5)c5sc6ccccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)sc2c(N(c4ccc5c(ccc6ccccc65)c4)c4ccc(-c5ccccc5)c5sc6ccccc6c45)cccc23)cc1]

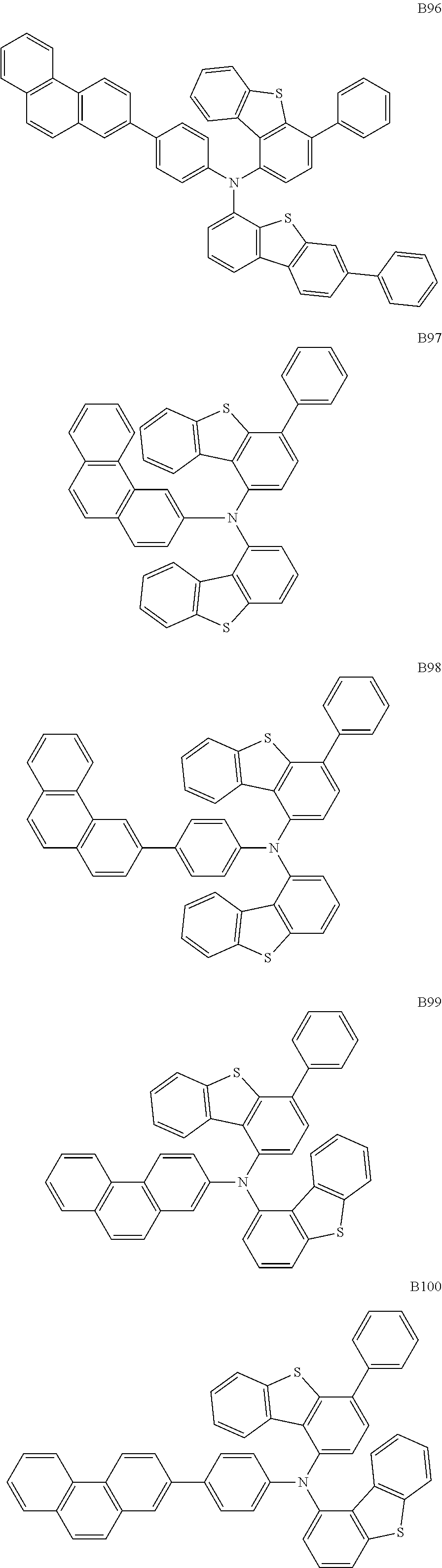 [SMILES: c1ccc(-c2ccc3c(c2)sc2c(N(c4ccc(-c5ccc6c(ccc7ccccc76)c5)cc4)c4ccc(-c5ccccc5)c5sc6ccccc6c45)cccc23)cc1 ; c1ccc(-c2ccc(N(c3ccc4ccc5ccccc5c4c3)c3cccc4sc5ccccc5c34)c3c2sc2ccccc23)cc1 ; c1ccc(-c2ccc(N(c3ccc(-c4ccc5ccc6ccccc6c5c4)cc3)c3cccc4sc5ccccc5c34)c3c2sc2ccccc23)cc1 ; c1ccc(-c2ccc(N(c3ccc4c(ccc5ccccc54)c3)c3cccc4sc5ccccc5c34)c3c2sc2ccccc23)cc1 ; c1ccc(-c2ccc(N(c3ccc(-c4ccc5c(ccc6ccccc65)c4)cc3)c3cccc4sc5ccccc5c34)c3c2sc2ccccc23)cc1]

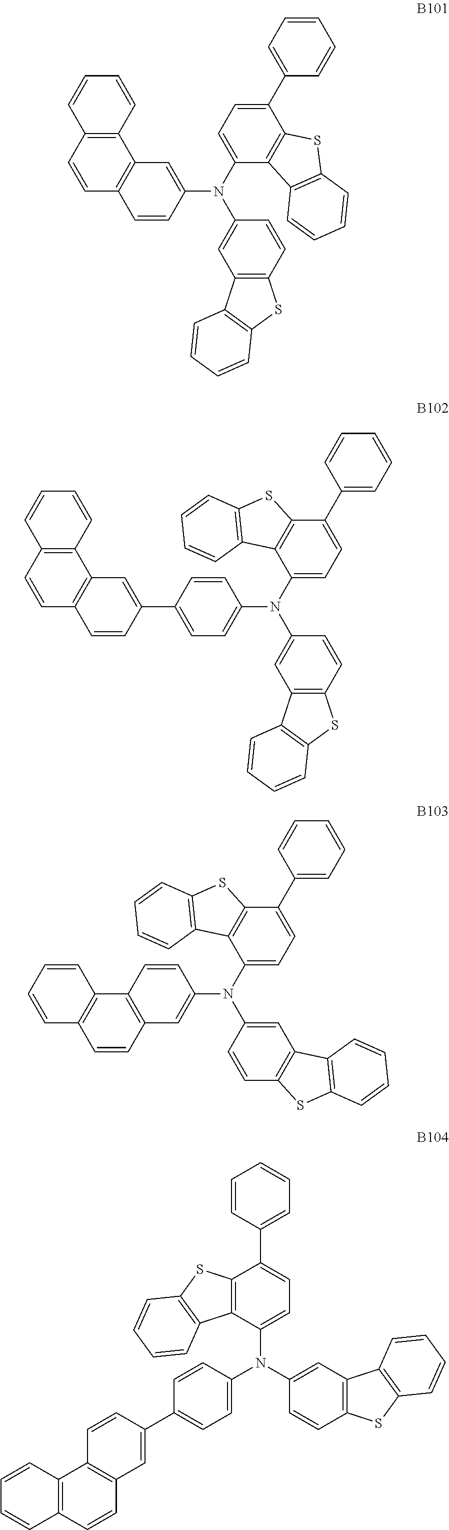

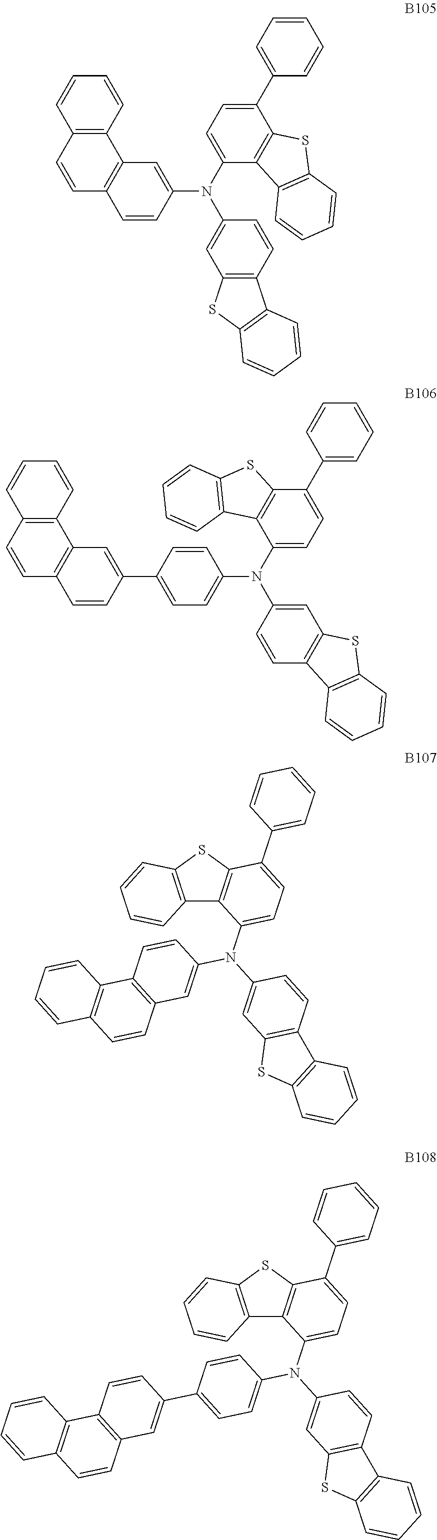 [SMILES: c1ccc(-c2ccc(N(c3ccc4c(c3)sc3ccccc34)c3ccc4ccc5ccccc5c4c3)c3c2sc2ccccc23)cc1 ; c1ccc(-c2ccc(N(c3ccc(-c4ccc5ccc6ccccc6c5c4)cc3)c3ccc4c(c3)sc3ccccc34)c3c2sc2ccccc23)cc1 ; c1ccc(-c2ccc(N(c3ccc4c(ccc5ccccc54)c3)c3ccc4c(c3)sc3ccccc34)c3c2sc2ccccc23)cc1 ; c1ccc(-c2ccc(N(c3ccc(-c4ccc5c(ccc6ccccc65)c4)cc3)c3ccc4c(c3)sc3ccccc34)c3c2sc2ccccc23)cc1]

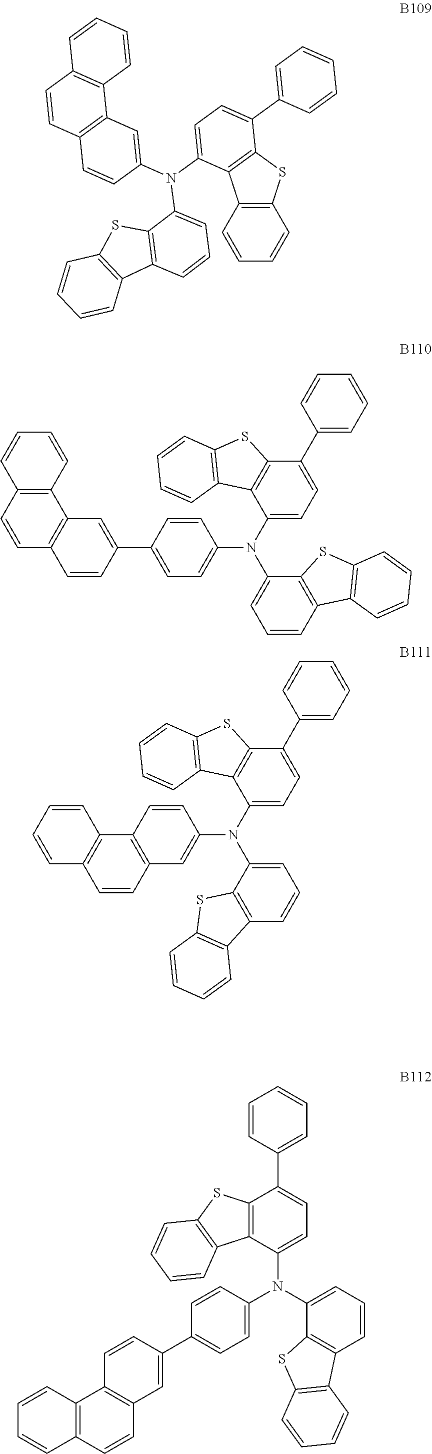

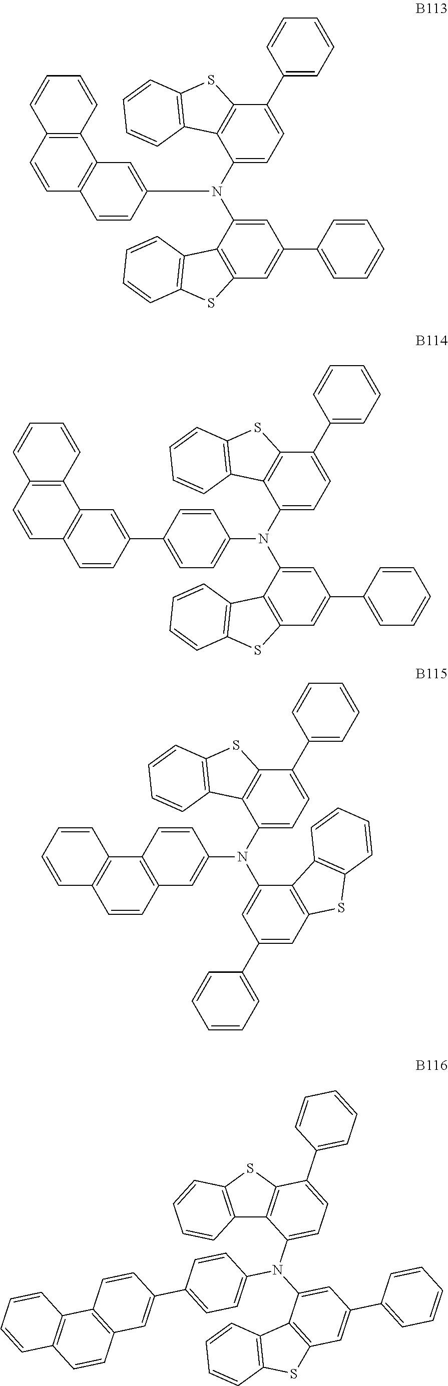 [SMILES: c1ccc(-c2cc(N(c3ccc4ccc5ccccc5c4c3)c3ccc(-c4ccccc4)c4sc5ccccc5c34)c3c(c2)sc2ccccc23)cc1 ; c1ccc(-c2cc(N(c3ccc(-c4ccc5ccc6ccccc6c5c4)cc3)c3ccc(-c4ccccc4)c4sc5ccccc5c34)c3c(c2)sc2ccccc23)cc1 ; c1ccc(-c2cc(N(c3ccc4c(ccc5ccccc54)c3)c3ccc(-c4ccccc4)c4sc5ccccc5c34)c3c(c2)sc2ccccc23)cc1 ; c1ccc(-c2cc(N(c3ccc(-c4ccc5c(ccc6ccccc65)c4)cc3)c3ccc(-c4ccccc4)c4sc5ccccc5c34)c3c(c2)sc2ccccc23)cc1]

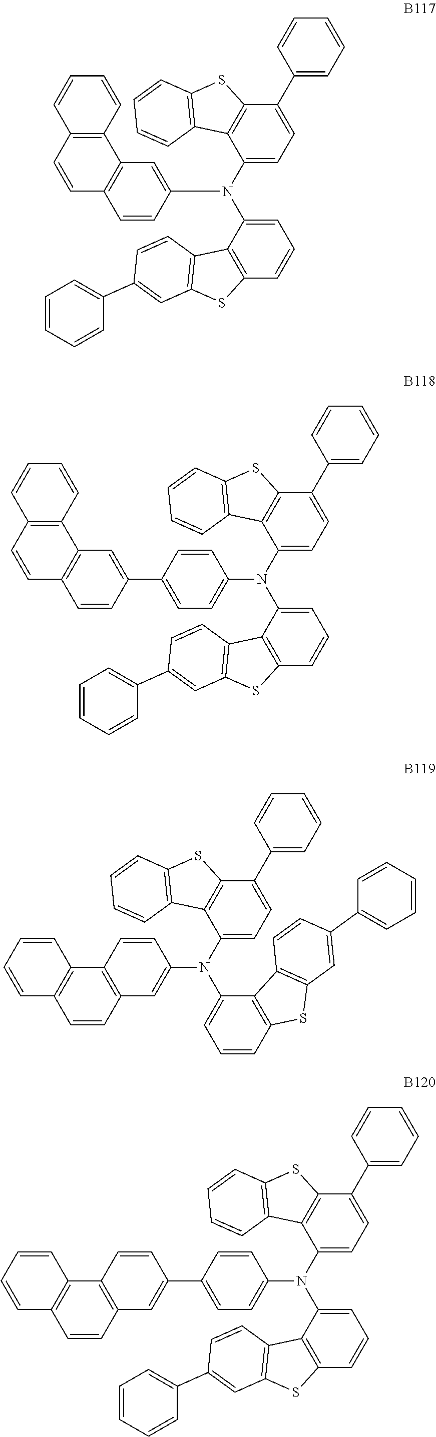 [SMILES: c1ccc(-c2ccc3c(c2)sc2cccc(N(c4ccc5ccc6ccccc6c5c4)c4ccc(-c5ccccc5)c5sc6ccccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5sc6ccccc6c45)c23)cc1 ; c1ccc(-c2ccc3c(c2)sc2cccc(N(c4ccc5c(ccc6ccccc65)c4)c4ccc(-c5ccccc5)c5sc6ccccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5sc6ccccc6c45)c23)cc1]

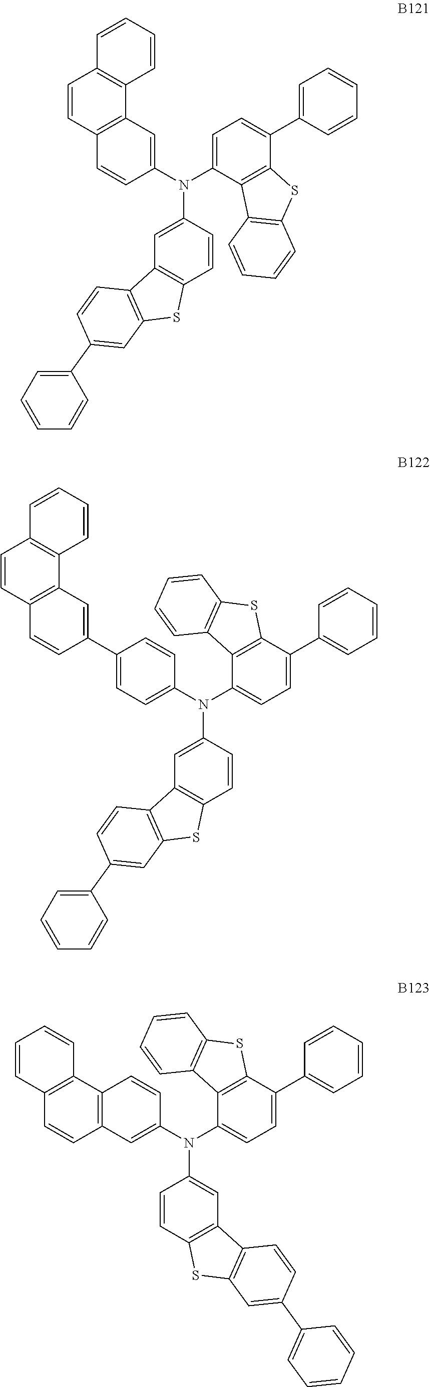 [SMILES: c1ccc(-c2ccc3c(c2)sc2ccc(N(c4ccc5ccc6ccccc6c5c4)c4ccc(-c5ccccc5)c5sc6ccccc6c45)cc23)cc1 ; c1ccc(-c2ccc3c(c2)sc2ccc(N(c4ccc(-c5ccc6ccc7ccccc7c6c5)cc4)c4ccc(-c5ccccc5)c5sc6ccccc6c45)cc23)cc1 ; c1ccc(-c2ccc3c(c2)sc2ccc(N(c4ccc5c(ccc6ccccc65)c4)c4ccc(-c5ccccc5)c5sc6ccccc6c45)cc23)cc1]

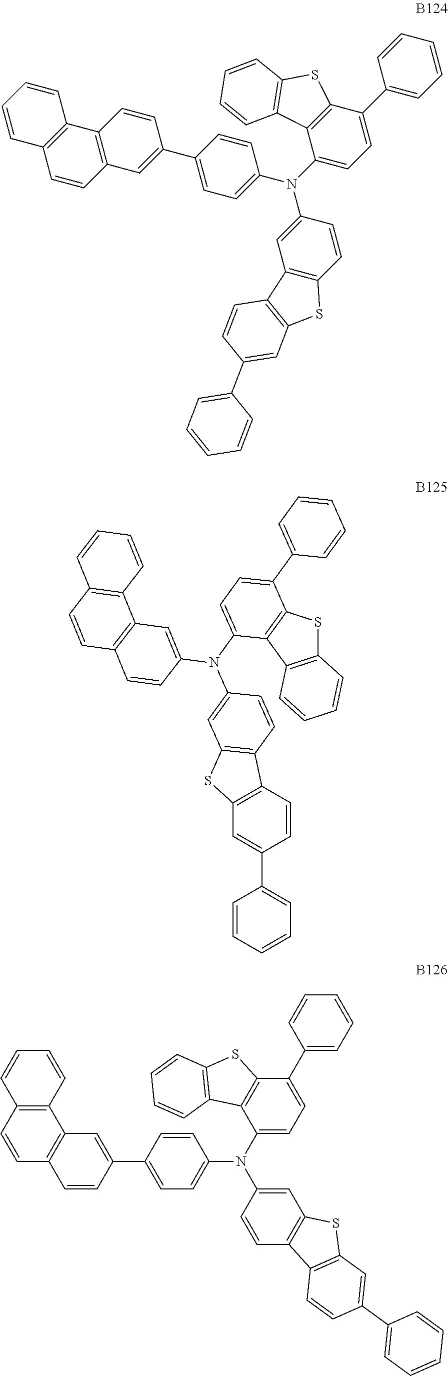 [SMILES: c1ccc(-c2ccc3c(c2)sc2ccc(N(c4ccc(-c5ccc6c(ccc7ccccc76)c5)cc4)c4ccc(-c5ccccc5)c5sc6ccccc6c45)cc23)cc1 ; c1ccc(-c2ccc3c(c2)sc2cc(N(c4ccc5ccc6ccccc6c5c4)c4ccc(-c5ccccc5)c5sc6ccccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5sc6ccccc6c45)ccc23)cc1]

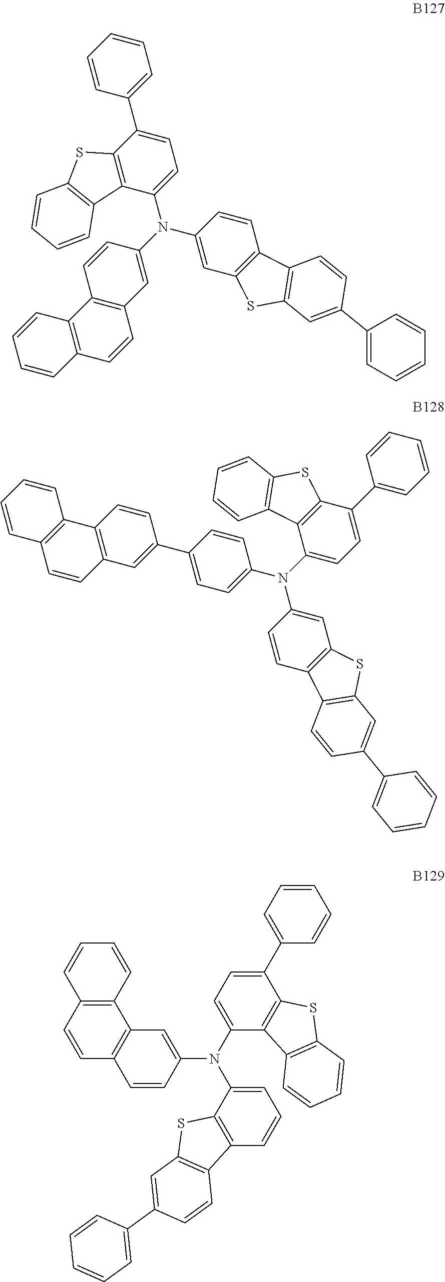 [SMILES: c1ccc(-c2ccc3c(c2)sc2cc(N(c4ccc5c(ccc6ccccc65)c4)c4ccc(-c5ccccc5)c5sc6ccccc6c45)ccc23)cc1 ; c1ccc(-c2ccc3c(c2)sc2cc(N(c4ccc(-c5ccc6c(ccc7ccccc76)c5)cc4)c4ccc(-c5ccccc5)c5sc6ccccc6c45)ccc23)cc1 ; c1ccc(-c2ccc3c(c2)sc2c(N(c4ccc5ccc6ccccc6c5c4)c4ccc(-c5ccccc5)c5sc6ccccc6c45)cccc23)cc1]

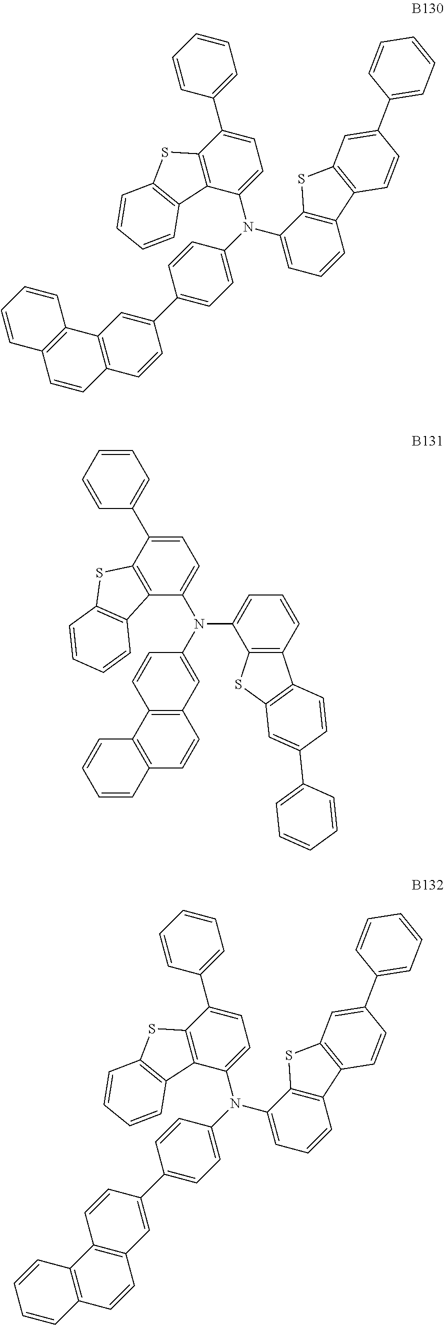 [SMILES: c1ccc(-c2ccc3c(c2)sc2c(N(c4ccc(-c5ccc6ccc7ccccc7c6c5)cc4)c4ccc(-c5ccccc5)c5sc6ccccc6c45)cccc23)cc1 ; c1ccc(-c2ccc3c(c2)sc2c(N(c4ccc5c(ccc6ccccc65)c4)c4ccc(-c5ccccc5)c5sc6ccccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5sc6ccccc6c45)cccc23)cc1]

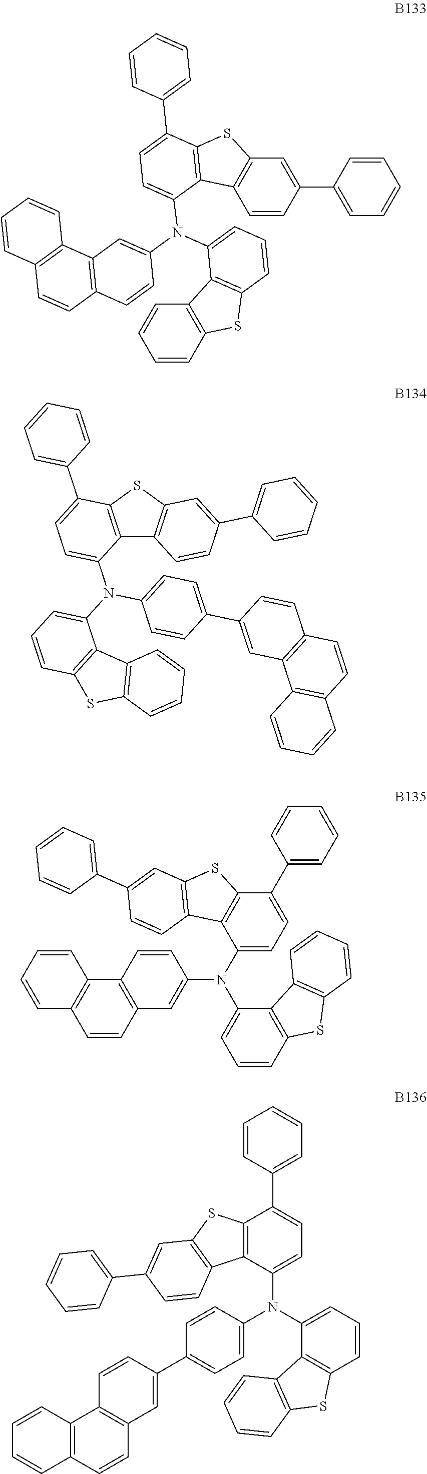

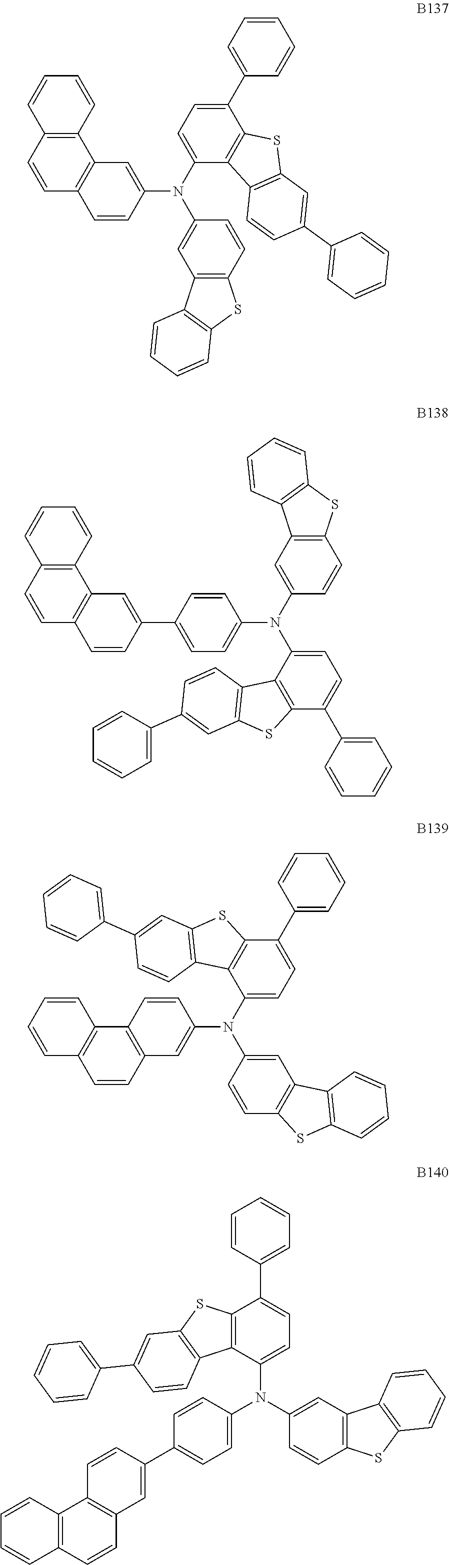 [SMILES: c1ccc(-c2ccc3c(c2)sc2c(-c4ccccc4)ccc(N(c4ccc5ccc6ccccc6c5c4)c4ccc5sc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5sc6ccccc6c5c4)c23)cc1 ; c1ccc(-c2ccc3c(c2)sc2c(-c4ccccc4)ccc(N(c4ccc5c(ccc6ccccc65)c4)c4ccc5sc6ccccc6c5c4)c23)cc1 ; c1ccc(-c2ccc3c(c2)sc2c(-c4ccccc4)ccc(N(c4ccc(-c5ccc6c(ccc7ccccc76)c5)cc4)c4ccc5sc6ccccc6c5c4)c23)cc1]

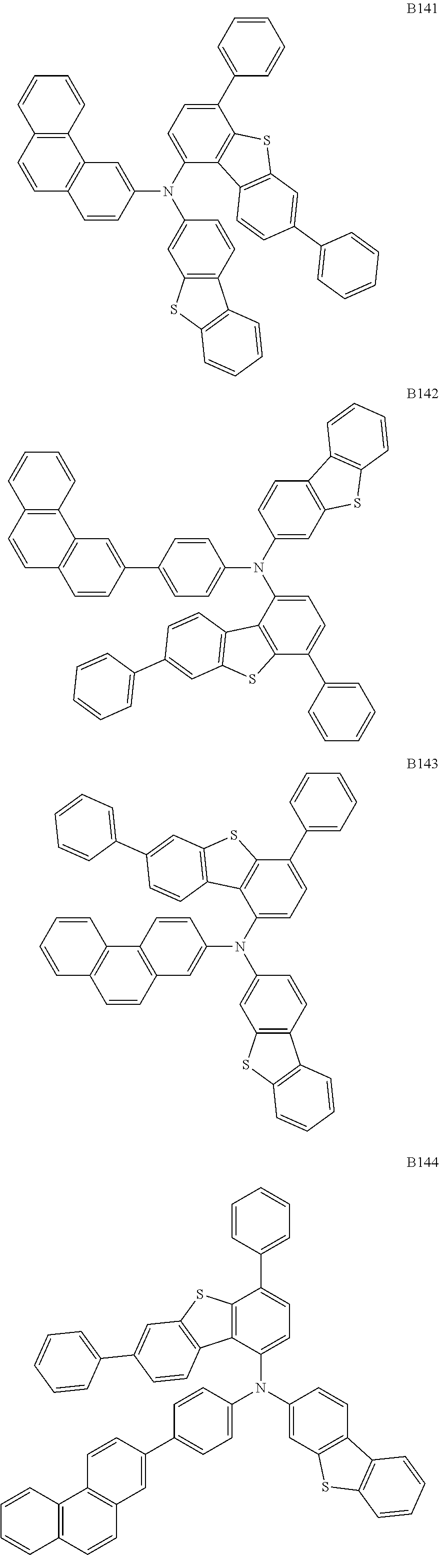

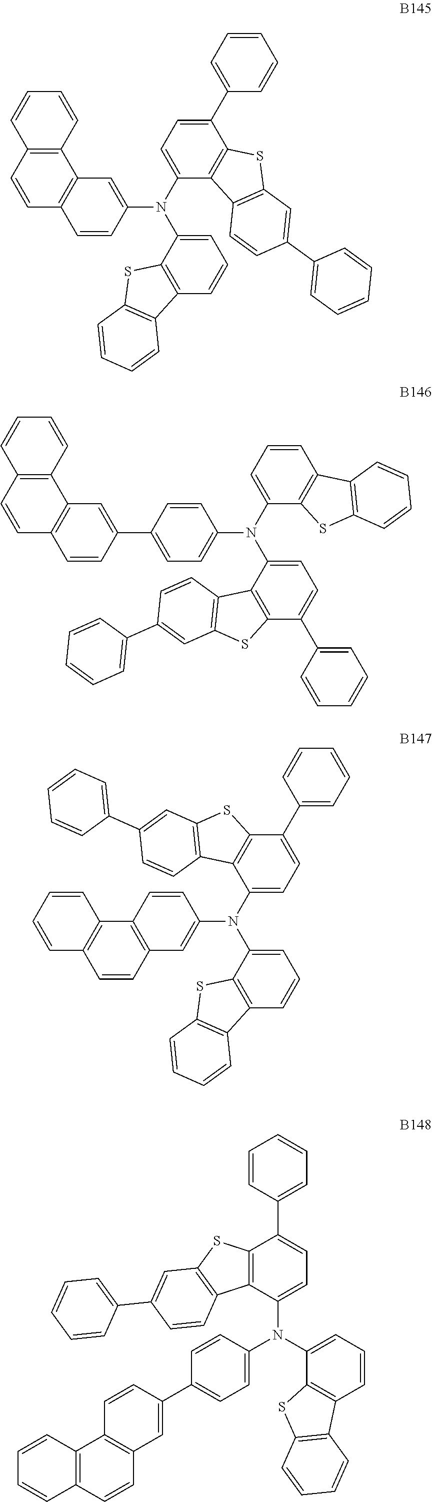

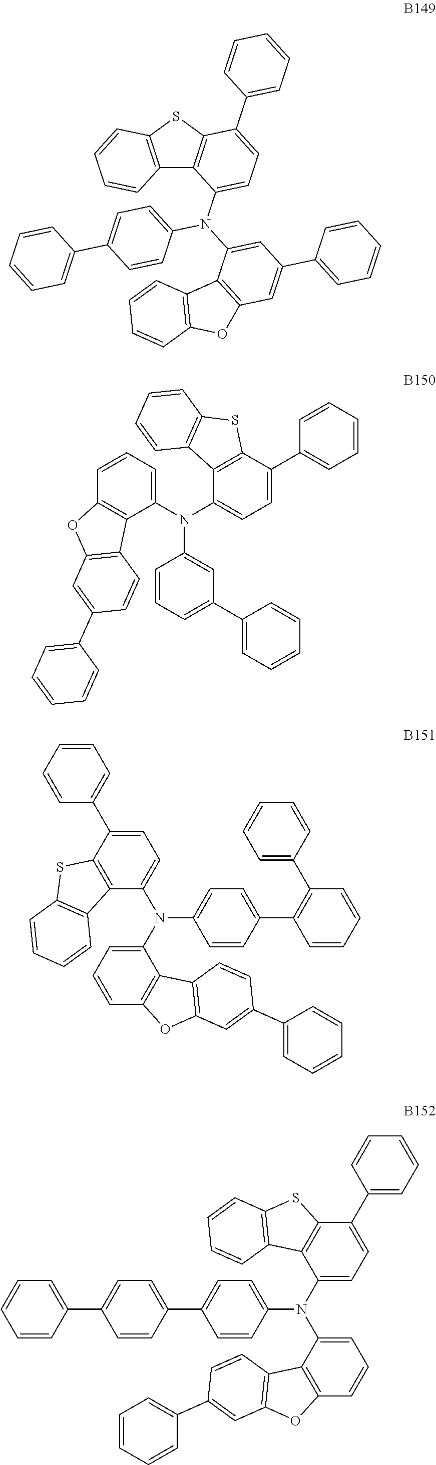

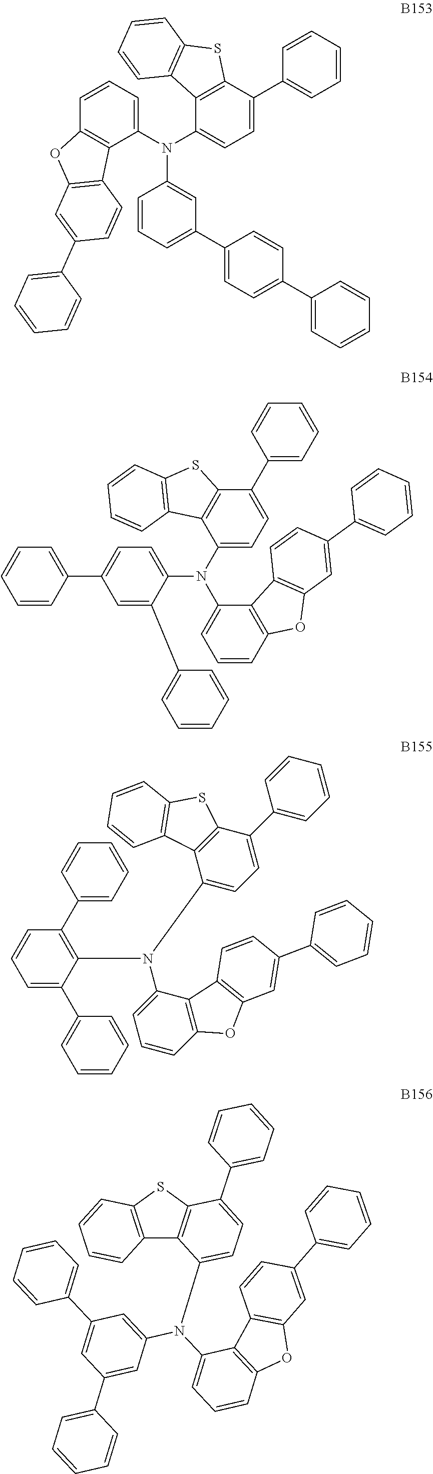 [SMILES: c1ccc(-c2ccc(-c3cccc(N(c4cccc5oc6cc(-c7ccccc7)ccc6c45)c4ccc(-c5ccccc5)c5sc6ccccc6c45)c3)cc2)cc1 ; c1ccc(-c2ccc(N(c3cccc4oc5cc(-c6ccccc6)ccc5c34)c3ccc(-c4ccccc4)c4sc5ccccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5sc6ccccc6c45)c23)cc1 ; c1ccc(-c2cc(-c3ccccc3)cc(N(c3cccc4oc5cc(-c6ccccc6)ccc5c34)c3ccc(-c4ccccc4)c4sc5ccccc5c34)c2)cc1]

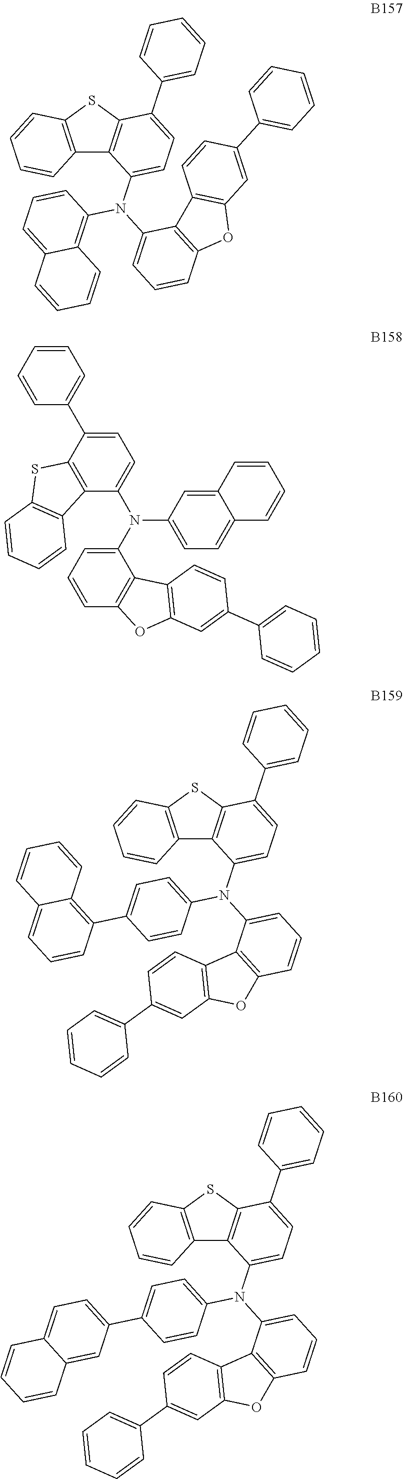

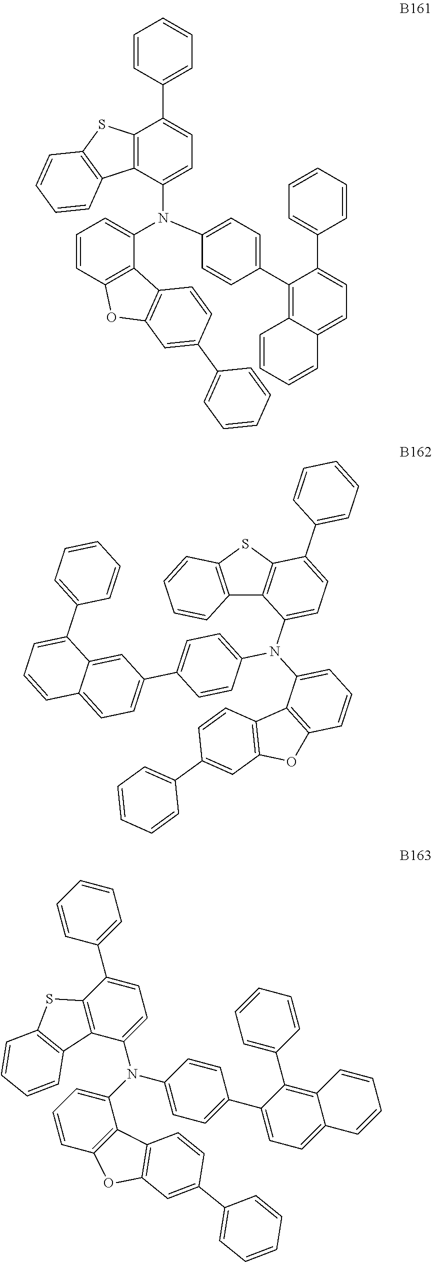 [SMILES: c1ccc(-c2ccc3c(c2)oc2cccc(N(c4ccc(-c5c(-c6ccccc6)ccc6ccccc56)cc4)c4ccc(-c5ccccc5)c5sc6ccccc6c45)c23)cc1 ; c1ccc(-c2ccc3c(c2)oc2cccc(N(c4ccc(-c5ccc6cccc(-c7ccccc7)c6c5)cc4)c4ccc(-c5ccccc5)c5sc6ccccc6c45)c23)cc1 ; c1ccc(-c2ccc3c(c2)oc2cccc(N(c4ccc(-c5ccc6ccccc6c5-c5ccccc5)cc4)c4ccc(-c5ccccc5)c5sc6ccccc6c45)c23)cc1]

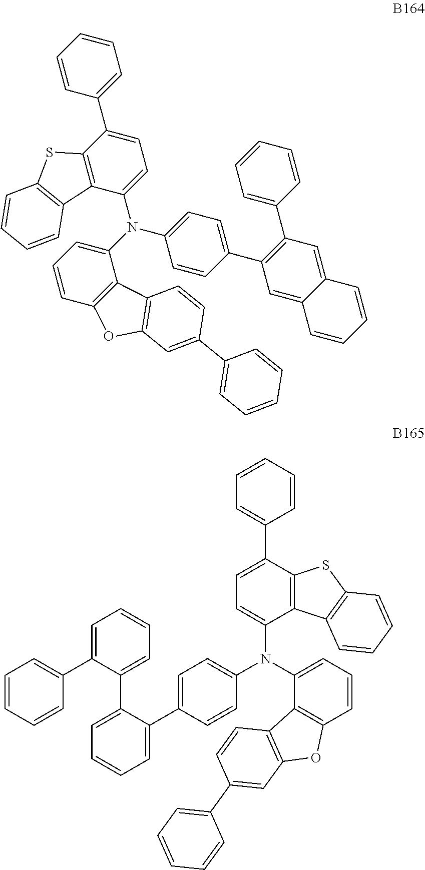 [SMILES: c1ccc(-c2ccc3c(c2)oc2cccc(N(c4ccc(-c5cc6ccccc6cc5-c5ccccc5)cc4)c4ccc(-c5ccccc5)c5sc6ccccc6c45)c23)cc1 ; c1ccc(-c2ccc3c(c2)oc2cccc(N(c4ccc(-c5ccccc5-c5ccccc5-c5ccccc5)cc4)c4ccc(-c5ccccc5)c5sc6ccccc6c45)c23)cc1]

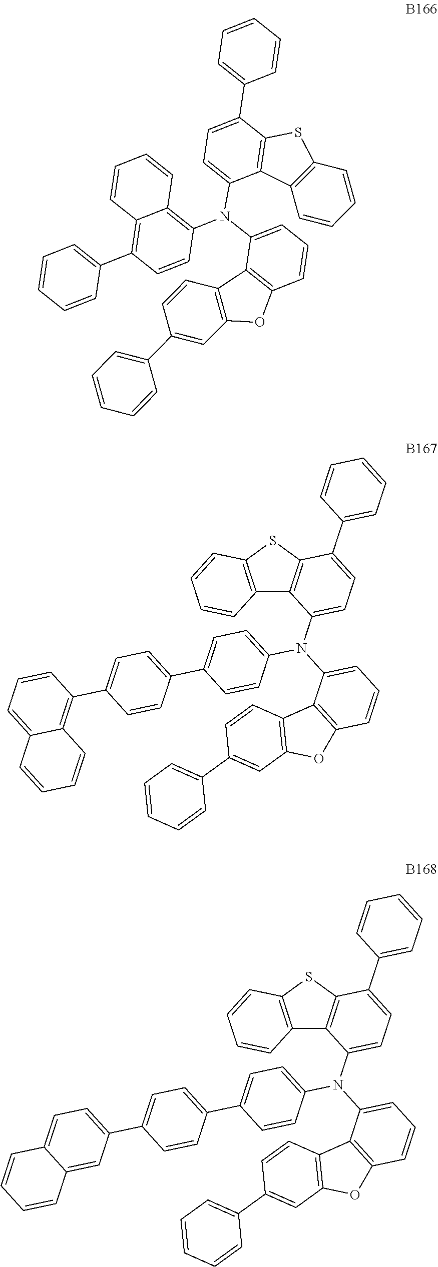 [SMILES: c1ccc(-c2ccc3c(c2)oc2cccc(N(c4ccc(-c5ccccc5)c5ccccc45)c4ccc(-c5ccccc5)c5sc6ccccc6c45)c23)cc1 ; c1ccc(-c2ccc3c(c2)oc2cccc(N(c4ccc(-c5ccc(-c6cccc7ccccc67)cc5)cc4)c4ccc(-c5ccccc5)c5sc6ccccc6c45)c23)cc1 ; c1ccc(-c2ccc3c(c2)oc2cccc(N(c4ccc(-c5ccc(-c6ccc7ccccc7c6)cc5)cc4)c4ccc(-c5ccccc5)c5sc6ccccc6c45)c23)cc1]

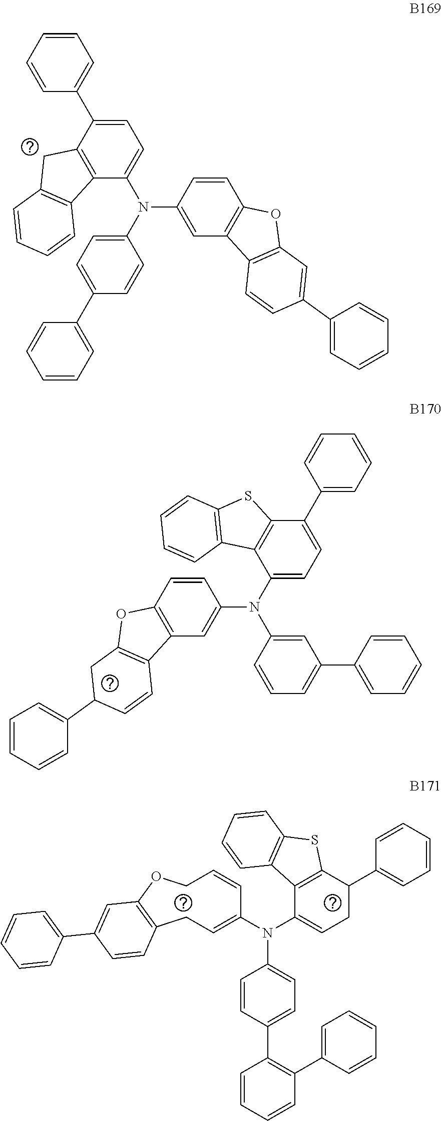 [SMILES: c1ccc(-c2ccc(N(c3ccc4oc5cc(-c6ccccc6)ccc5c4c3)c3ccc(-c4ccccc4)c4c3-c3ccccc3C4)cc2)cc1 ; C1=CC(c2ccccc2)Cc2oc3ccc(N(c4cccc(-c5ccccc5)c4)c4ccc(-c5ccccc5)c5sc6ccccc6c45)cc3c21 ; C1=C(N(C2=C/Cc3ccc(-c4ccccc4)cc3OC/C=C\2)c2ccc(-c3ccccc3-c3ccccc3)cc2)c2c(sc3ccccc23)C(c2ccccc2)C1]

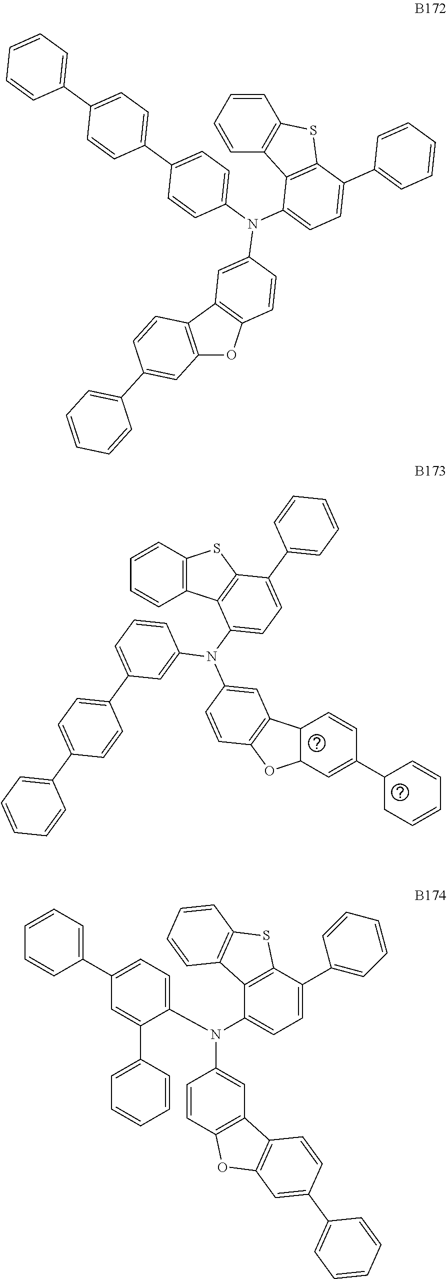 [SMILES: c1ccc(-c2ccc(-c3ccc(N(c4ccc5oc6cc(-c7ccccc7)ccc6c5c4)c4ccc(-c5ccccc5)c5sc6ccccc6c45)cc3)cc2)cc1 ; C1=CCC(C2=CC3Oc4ccc(N(c5cccc(-c6ccc(-c7ccccc7)cc6)c5)c5ccc(-c6ccccc6)c6sc7ccccc7c56)cc4C3C=C2)C=C1 ; c1ccc(-c2ccc(N(c3ccc4oc5cc(-c6ccccc6)ccc5c4c3)c3ccc(-c4ccccc4)c4sc5ccccc5c34)c(-c3ccccc3)c2)cc1]

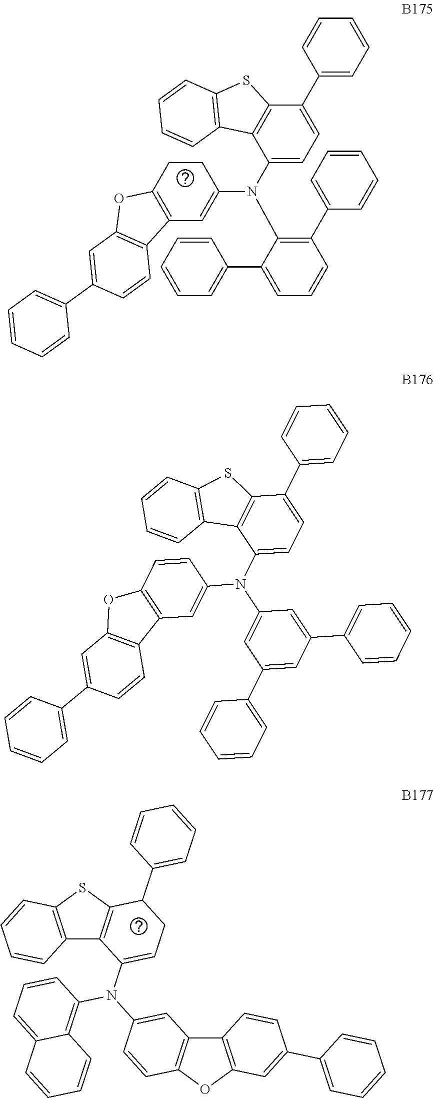 [SMILES: C1=C(N(c2c(-c3ccccc3)cccc2-c2ccccc2)c2ccc(-c3ccccc3)c3sc4ccccc4c23)CCc2oc3cc(-c4ccccc4)ccc3c21 ; c1ccc(-c2cc(-c3ccccc3)cc(N(c3ccc4oc5cc(-c6ccccc6)ccc5c4c3)c3ccc(-c4ccccc4)c4sc5ccccc5c34)c2)cc1 ; C1=C(N(c2ccc3oc4cc(-c5ccccc5)ccc4c3c2)c2cccc3ccccc23)c2c(sc3ccccc23)C(c2ccccc2)C1]

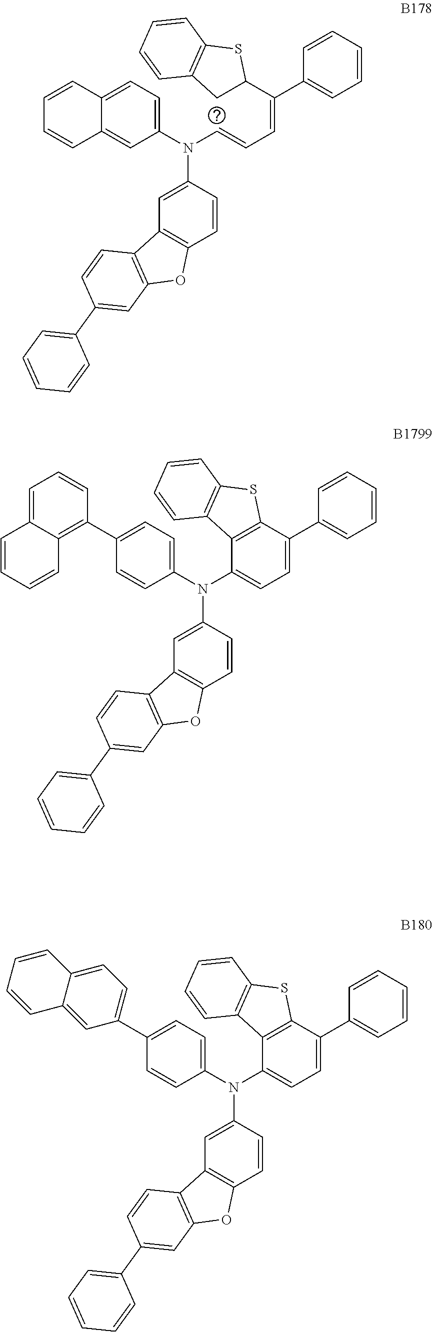 [SMILES: C(/C=C/N(c1ccc2ccccc2c1)c1ccc2oc3cc(-c4ccccc4)ccc3c2c1)=C(\c1ccccc1)C1Cc2ccccc2S1 ; c1ccc(-c2ccc3c(c2)oc2ccc(N(c4ccc(-c5cccc6ccccc56)cc4)c4ccc(-c5ccccc5)c5sc6ccccc6c45)cc23)cc1 ; c1ccc(-c2ccc3c(c2)oc2ccc(N(c4ccc(-c5ccc6ccccc6c5)cc4)c4ccc(-c5ccccc5)c5sc6ccccc6c45)cc23)cc1]

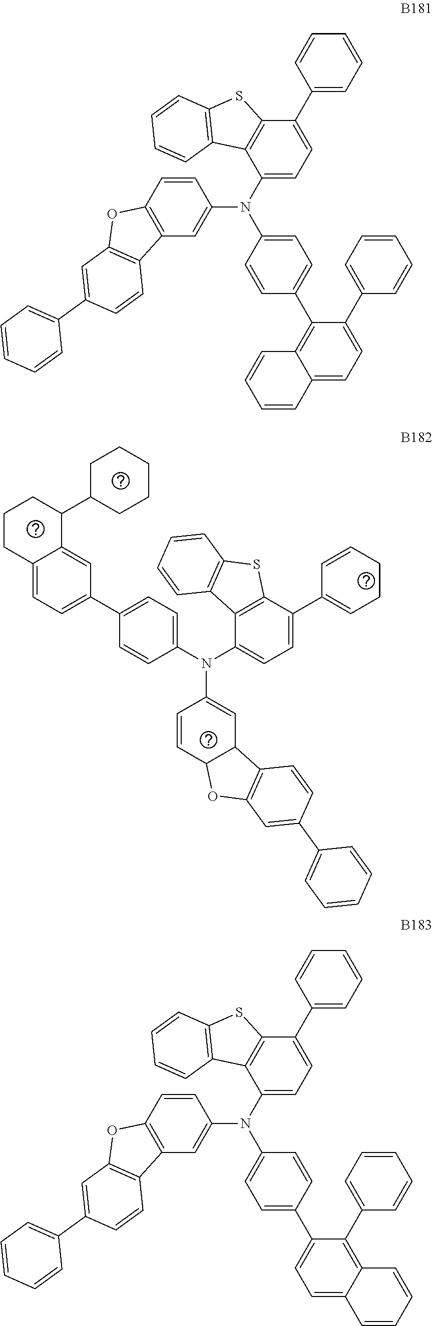

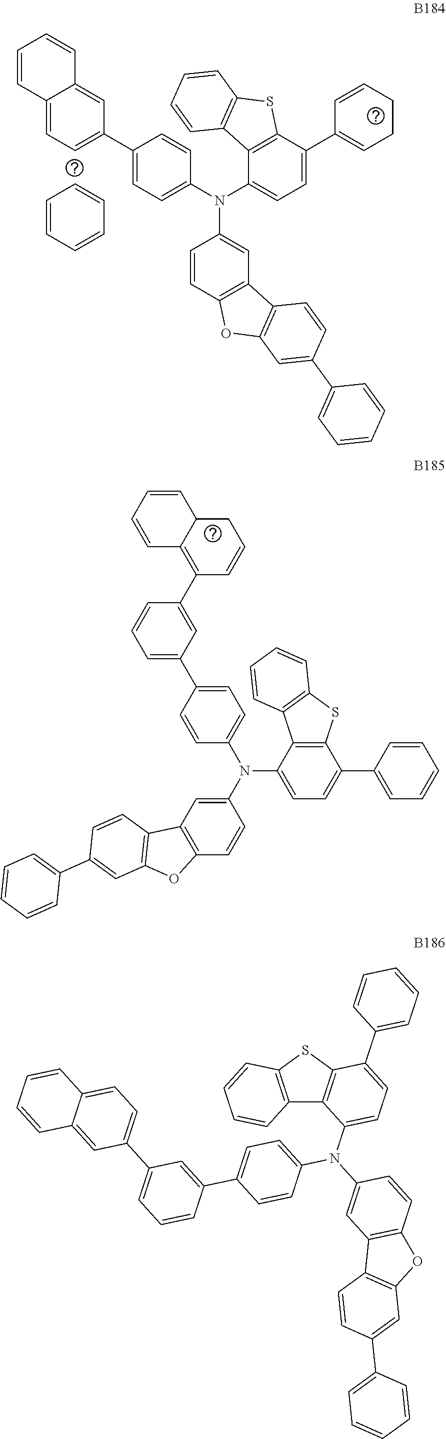

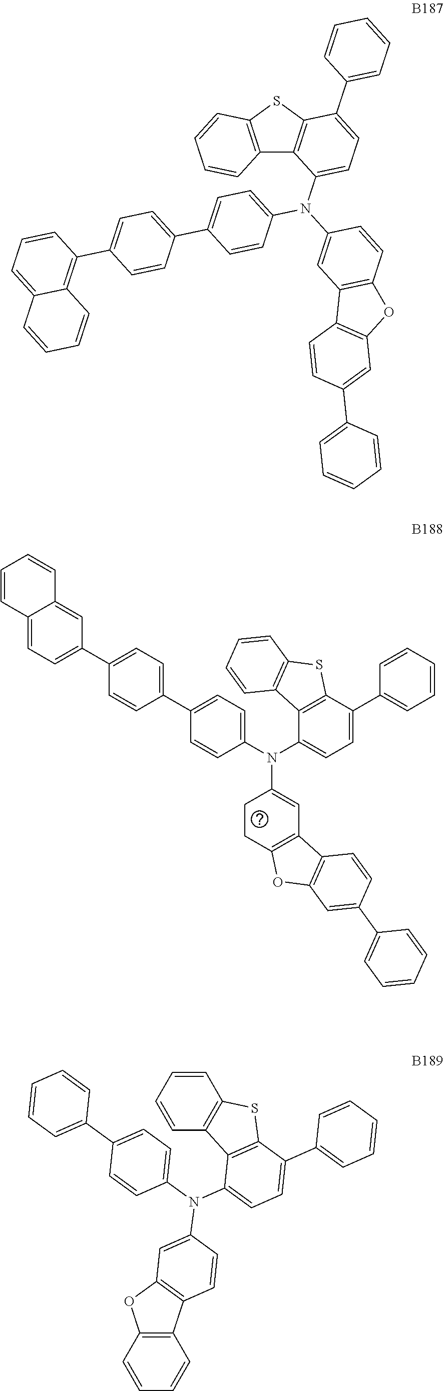 [SMILES: c1ccc(-c2ccc3c(c2)oc2ccc(N(c4ccc(-c5ccc(-c6cccc7ccccc67)cc5)cc4)c4ccc(-c5ccccc5)c5sc6ccccc6c45)cc23)cc1 ; C1=C(N(c2ccc(-c3ccc(-c4ccc5ccccc5c4)cc3)cc2)c2ccc(-c3ccccc3)c3sc4ccccc4c23)CCc2oc3cc(-c4ccccc4)ccc3c21 ; c1ccc(-c2ccc(N(c3ccc4c(c3)oc3ccccc34)c3ccc(-c4ccccc4)c4sc5ccccc5c34)cc2)cc1]

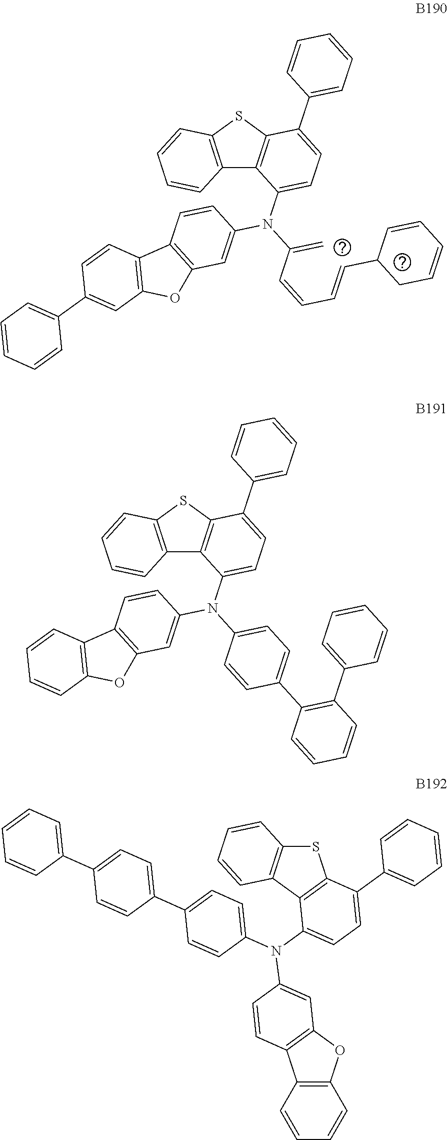 [SMILES: C=C(/C=C\C=C\C1C=CC=CC1)N(c1ccc2c(c1)oc1cc(-c3ccccc3)ccc12)c1ccc(-c2ccccc2)c2sc3ccccc3c12 ; c1ccc(-c2ccccc2-c2ccc(N(c3ccc4c(c3)oc3ccccc34)c3ccc(-c4ccccc4)c4sc5ccccc5c34)cc2)cc1 ; c1ccc(-c2ccc(-c3ccc(N(c4ccc5c(c4)oc4ccccc45)c4ccc(-c5ccccc5)c5sc6ccccc6c45)cc3)cc2)cc1]

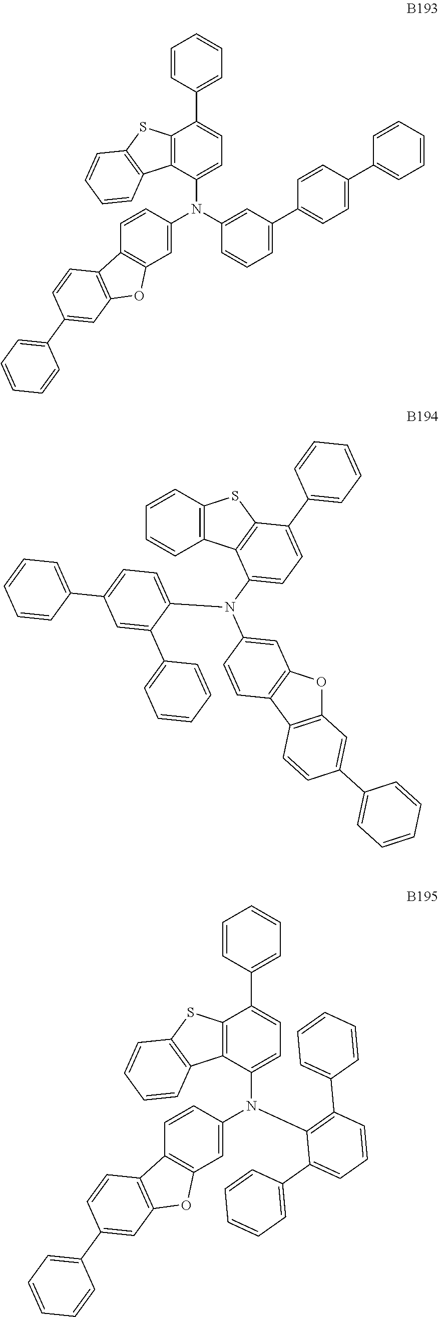 [SMILES: c1ccc(-c2ccc(-c3cccc(N(c4ccc5c(c4)oc4cc(-c6ccccc6)ccc45)c4ccc(-c5ccccc5)c5sc6ccccc6c45)c3)cc2)cc1 ; c1ccc(-c2ccc(N(c3ccc4c(c3)oc3cc(-c5ccccc5)ccc34)c3ccc(-c4ccccc4)c4sc5ccccc5c34)c(-c3ccccc3)c2)cc1 ; c1ccc(-c2ccc3c(c2)oc2cc(N(c4c(-c5ccccc5)cccc4-c4ccccc4)c4ccc(-c5ccccc5)c5sc6ccccc6c45)ccc23)cc1]

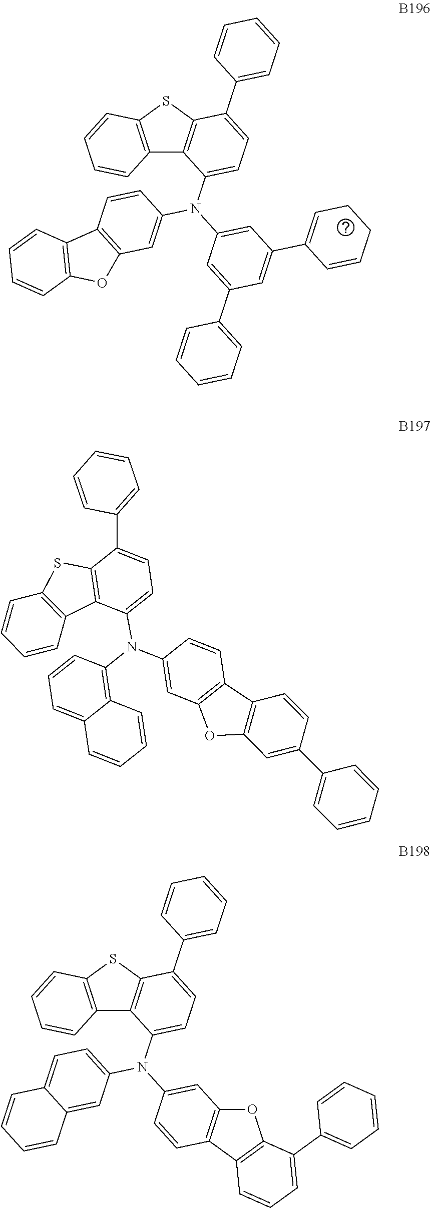 [SMILES: C1=CC(c2cc(-c3ccccc3)cc(N(c3ccc4c(c3)oc3ccccc34)c3ccc(-c4ccccc4)c4sc5ccccc5c34)c2)=CCC1 ; c1ccc(-c2ccc3c(c2)oc2cc(N(c4cccc5ccccc45)c4ccc(-c5ccccc5)c5sc6ccccc6c45)ccc23)cc1 ; c1ccc(-c2cccc3c2oc2cc(N(c4ccc5ccccc5c4)c4ccc(-c5ccccc5)c5sc6ccccc6c45)ccc23)cc1]

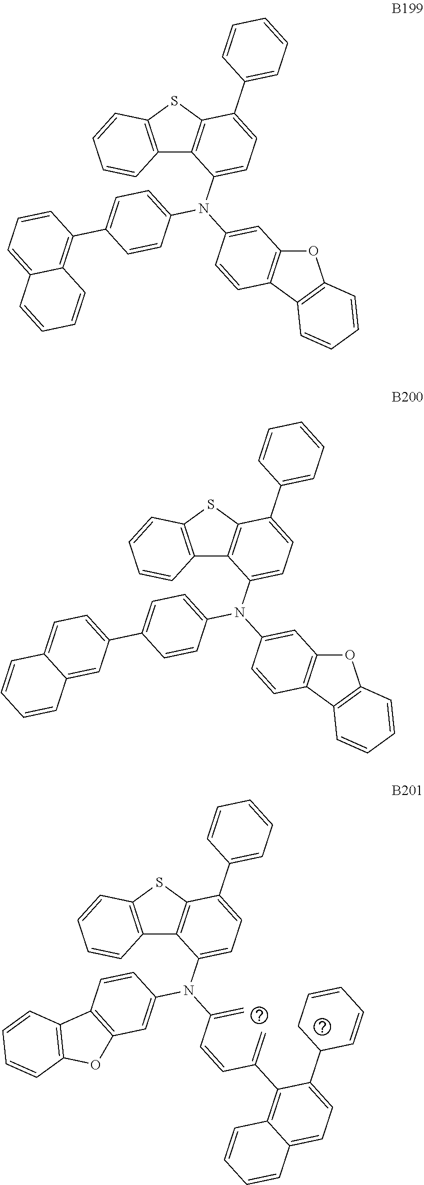 [SMILES: c1ccc(-c2ccc(N(c3ccc(-c4cccc5ccccc45)cc3)c3ccc4c(c3)oc3ccccc34)c3c2sc2ccccc23)cc1 ; c1ccc(-c2ccc(N(c3ccc(-c4ccc5ccccc5c4)cc3)c3ccc4c(c3)oc3ccccc34)c3c2sc2ccccc23)cc1 ; C=C(/C=C\C(=C)N(c1ccc2c(c1)oc1ccccc12)c1ccc(-c2ccccc2)c2sc3ccccc3c12)c1c(C2C=CC=CC2)ccc2ccccc12]

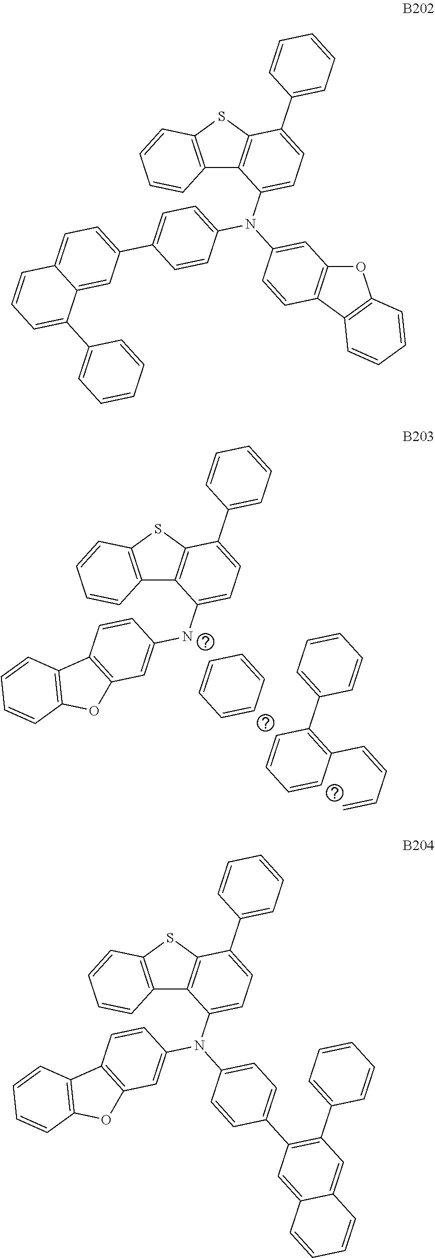

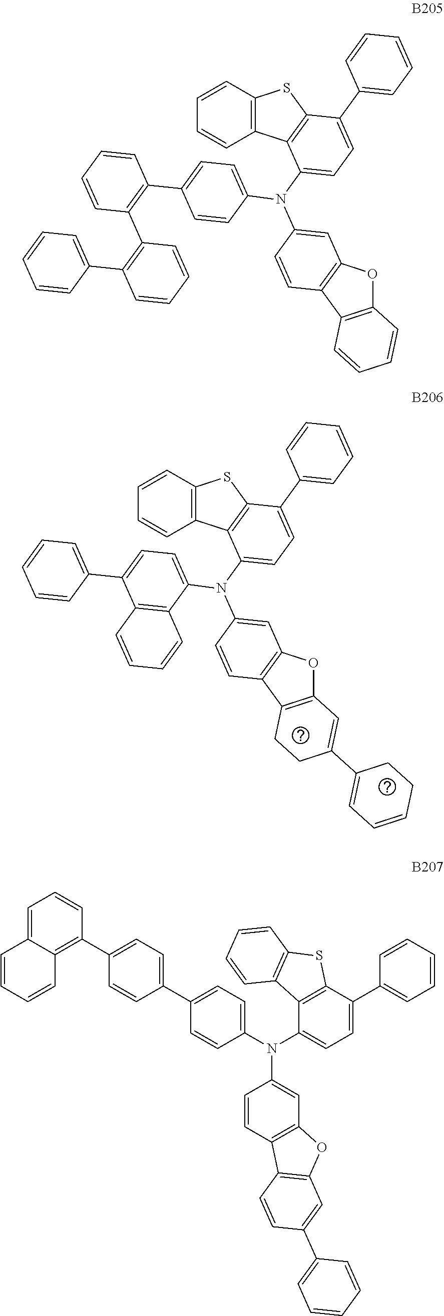 [SMILES: c1ccc(-c2ccccc2-c2ccccc2-c2ccc(N(c3ccc4c(c3)oc3ccccc34)c3ccc(-c4ccccc4)c4sc5ccccc5c34)cc2)cc1 ; C1=CCCC(C2=Cc3oc4cc(N(c5ccc(-c6ccccc6)c6ccccc56)c5ccc(-c6ccccc6)c6sc7ccccc7c56)ccc4c3CC2)=C1 ; c1ccc(-c2ccc3c(c2)oc2cc(N(c4ccc(-c5ccc(-c6cccc7ccccc67)cc5)cc4)c4ccc(-c5ccccc5)c5sc6ccccc6c45)ccc23)cc1]

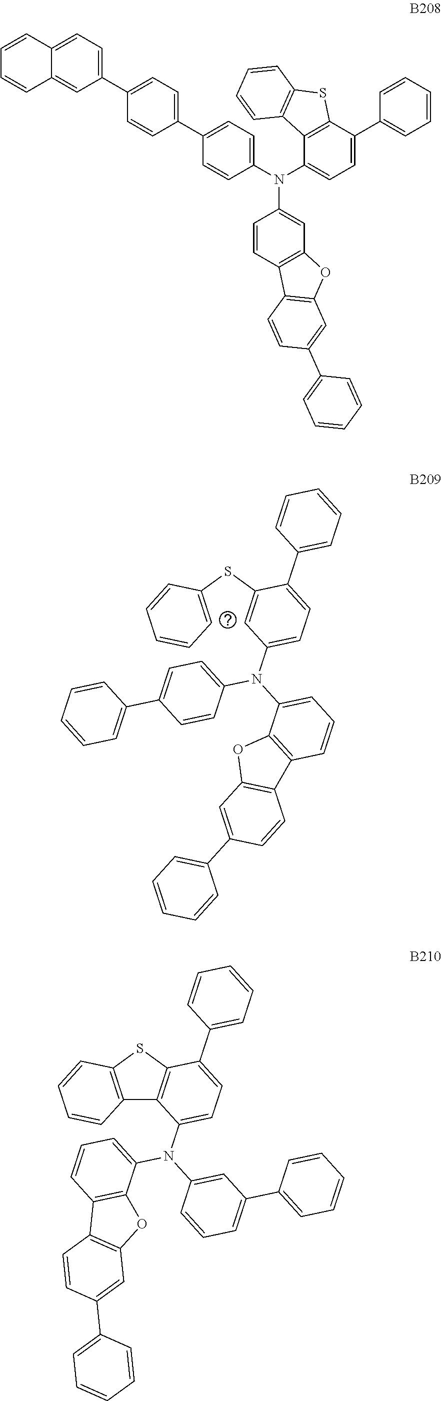 [SMILES: c1ccc(-c2ccc3c(c2)oc2cc(N(c4ccc(-c5ccc(-c6ccc7ccccc7c6)cc5)cc4)c4ccc(-c5ccccc5)c5sc6ccccc6c45)ccc23)cc1 ; c1ccc(Sc2cc(N(c3ccc(-c4ccccc4)cc3)c3cccc4c3oc3cc(-c5ccccc5)ccc34)ccc2-c2ccccc2)cc1 ; c1ccc(-c2cccc(N(c3cccc4c3oc3cc(-c5ccccc5)ccc34)c3ccc(-c4ccccc4)c4sc5ccccc5c34)c2)cc1]

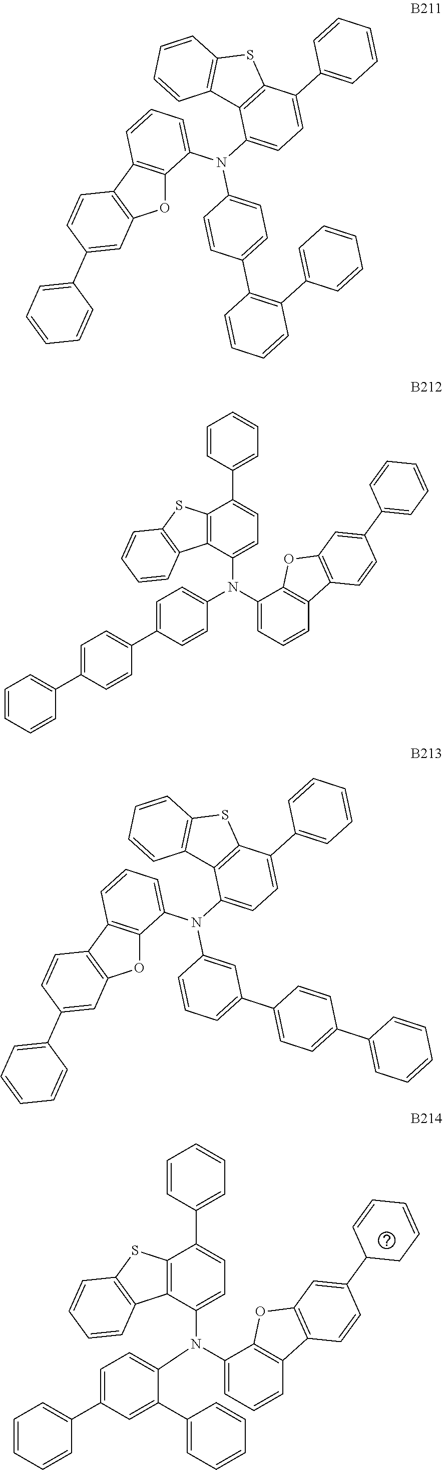 [SMILES: c1ccc(-c2ccc3c(c2)oc2c(N(c4ccc(-c5ccccc5-c5ccccc5)cc4)c4ccc(-c5ccccc5)c5sc6ccccc6c45)cccc23)cc1 ; c1ccc(-c2ccc(-c3ccc(N(c4cccc5c4oc4cc(-c6ccccc6)ccc45)c4ccc(-c5ccccc5)c5sc6ccccc6c45)cc3)cc2)cc1 ; c1ccc(-c2ccc(-c3cccc(N(c4cccc5c4oc4cc(-c6ccccc6)ccc45)c4ccc(-c5ccccc5)c5sc6ccccc6c45)c3)cc2)cc1 ; C1=CCC(c2ccc3c(c2)oc2c(N(c4ccc(-c5ccccc5)cc4-c4ccccc4)c4ccc(-c5ccccc5)c5sc6ccccc6c45)cccc23)C=C1]

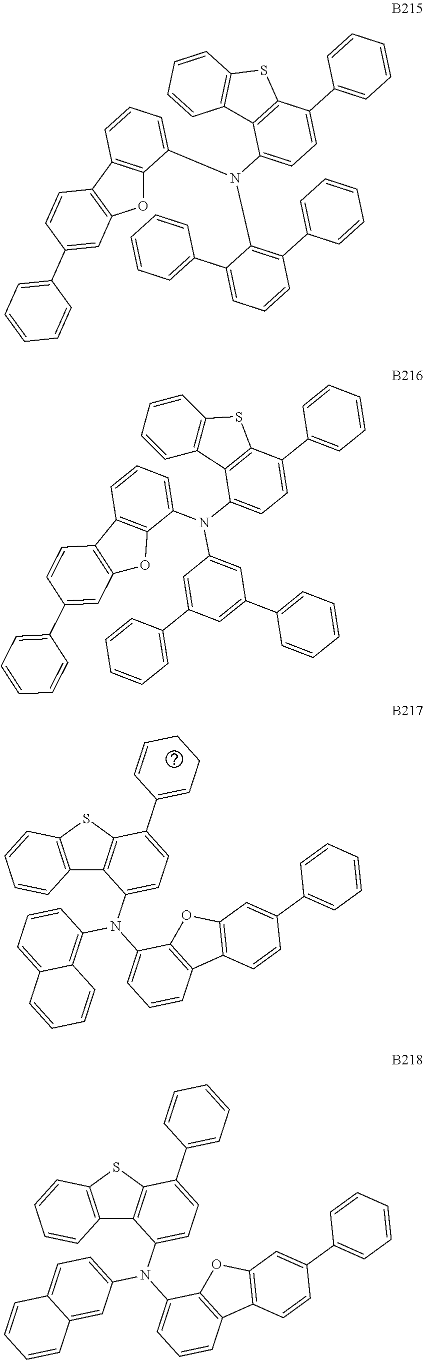 [SMILES: c1ccc(-c2ccc3c(c2)oc2c(N(c4c(-c5ccccc5)cccc4-c4ccccc4)c4ccc(-c5ccccc5)c5sc6ccccc6c45)cccc23)cc1 ; c1ccc(-c2cc(-c3ccccc3)cc(N(c3cccc4c3oc3cc(-c5ccccc5)ccc34)c3ccc(-c4ccccc4)c4sc5ccccc5c34)c2)cc1 ; C1=CC(c2ccc(N(c3cccc4ccccc34)c3cccc4c3oc3cc(-c5ccccc5)ccc34)c3c2sc2ccccc23)=CCC1 ; c1ccc(-c2ccc3c(c2)oc2c(N(c4ccc5ccccc5c4)c4ccc(-c5ccccc5)c5sc6ccccc6c45)cccc23)cc1]

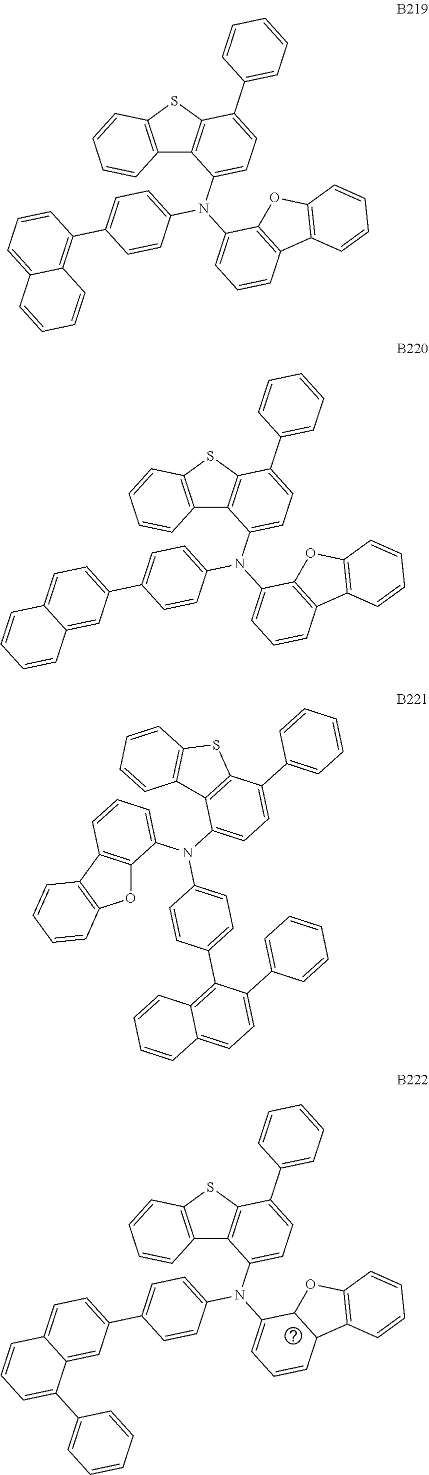 [SMILES: c1ccc(-c2ccc(N(c3ccc(-c4cccc5ccccc45)cc3)c3cccc4c3oc3ccccc34)c3c2sc2ccccc23)cc1 ; c1ccc(-c2ccc(N(c3ccc(-c4ccc5ccccc5c4)cc3)c3cccc4c3oc3ccccc34)c3c2sc2ccccc23)cc1 ; c1ccc(-c2ccc3ccccc3c2-c2ccc(N(c3cccc4c3oc3ccccc34)c3ccc(-c4ccccc4)c4sc5ccccc5c34)cc2)cc1 ; C1=CC2c3ccccc3OC2C(N(c2ccc(-c3ccc4cccc(-c5ccccc5)c4c3)cc2)c2ccc(-c3ccccc3)c3sc4ccccc4c23)=C1]

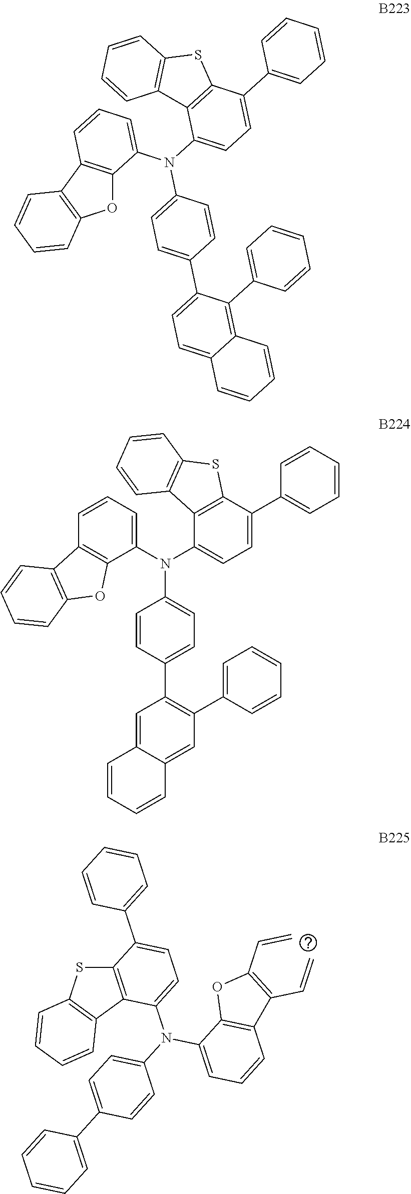

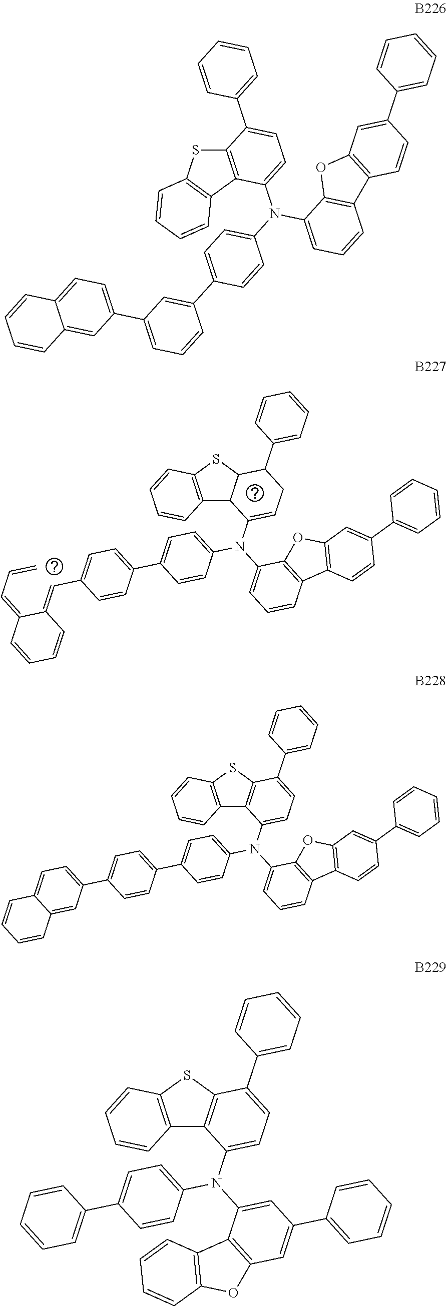 [SMILES: c1ccc(-c2ccc3c(c2)oc2c(N(c4ccc(-c5cccc(-c6ccc7ccccc7c6)c5)cc4)c4ccc(-c5ccccc5)c5sc6ccccc6c45)cccc23)cc1 ; C=C/C=c1/cccc/c1=C\c1ccc(-c2ccc(N(C3=CCC(c4ccccc4)C4Sc5ccccc5C34)c3cccc4c3oc3cc(-c5ccccc5)ccc34)cc2)cc1 ; c1ccc(-c2ccc3c(c2)oc2c(N(c4ccc(-c5ccc(-c6ccc7ccccc7c6)cc5)cc4)c4ccc(-c5ccccc5)c5sc6ccccc6c45)cccc23)cc1 ; c1ccc(-c2ccc(N(c3cc(-c4ccccc4)cc4oc5ccccc5c34)c3ccc(-c4ccccc4)c4sc5ccccc5c34)cc2)cc1]

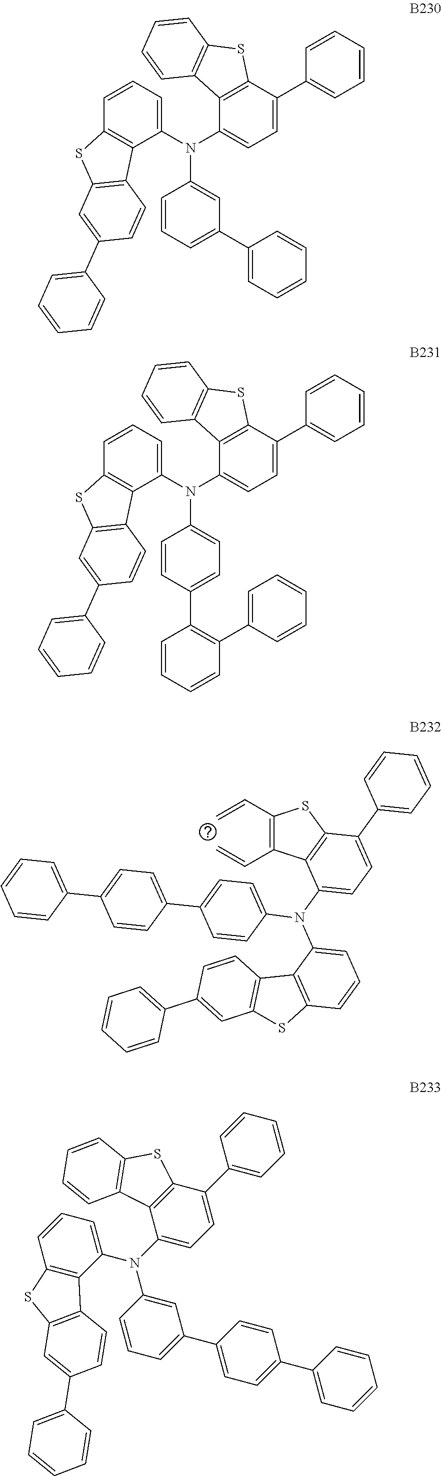 [SMILES: c1ccc(-c2cccc(N(c3cccc4sc5cc(-c6ccccc6)ccc5c34)c3ccc(-c4ccccc4)c4sc5ccccc5c34)c2)cc1 ; c1ccc(-c2ccc3c(c2)sc2cccc(N(c4ccc(-c5ccccc5-c5ccccc5)cc4)c4ccc(-c5ccccc5)c5sc6ccccc6c45)c23)cc1 ; C=Cc1sc2c(-c3ccccc3)ccc(N(c3ccc(-c4ccc(-c5ccccc5)cc4)cc3)c3cccc4sc5cc(-c6ccccc6)ccc5c34)c2c1C=C ; c1ccc(-c2ccc(-c3cccc(N(c4cccc5sc6cc(-c7ccccc7)ccc6c45)c4ccc(-c5ccccc5)c5sc6ccccc6c45)c3)cc2)cc1]

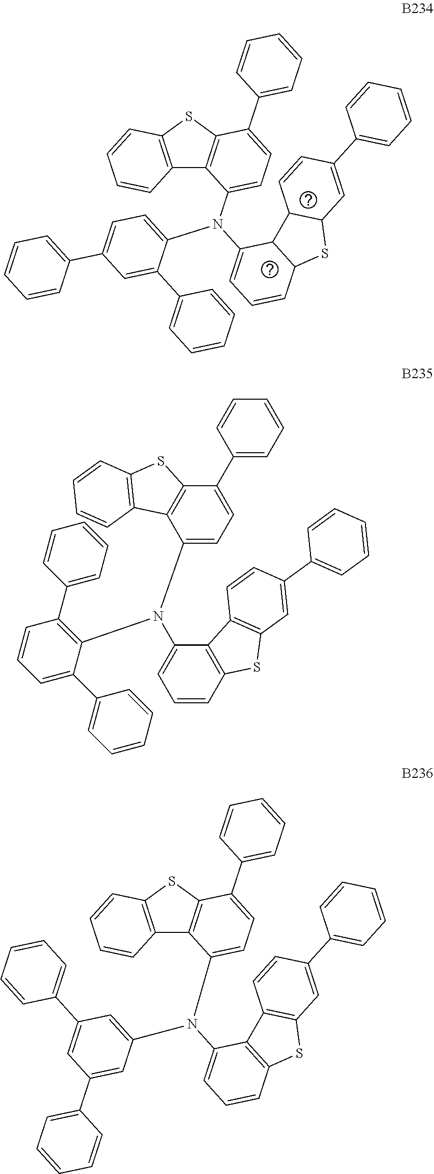 [SMILES: C1=CC2SC3C=C(c4ccccc4)C=CC3C2C(N(c2ccc(-c3ccccc3)cc2-c2ccccc2)c2ccc(-c3ccccc3)c3sc4ccccc4c23)=C1 ; c1ccc(-c2ccc3c(c2)sc2cccc(N(c4c(-c5ccccc5)cccc4-c4ccccc4)c4ccc(-c5ccccc5)c5sc6ccccc6c45)c23)cc1 ; c1ccc(-c2cc(-c3ccccc3)cc(N(c3cccc4sc5cc(-c6ccccc6)ccc5c34)c3ccc(-c4ccccc4)c4sc5ccccc5c34)c2)cc1]

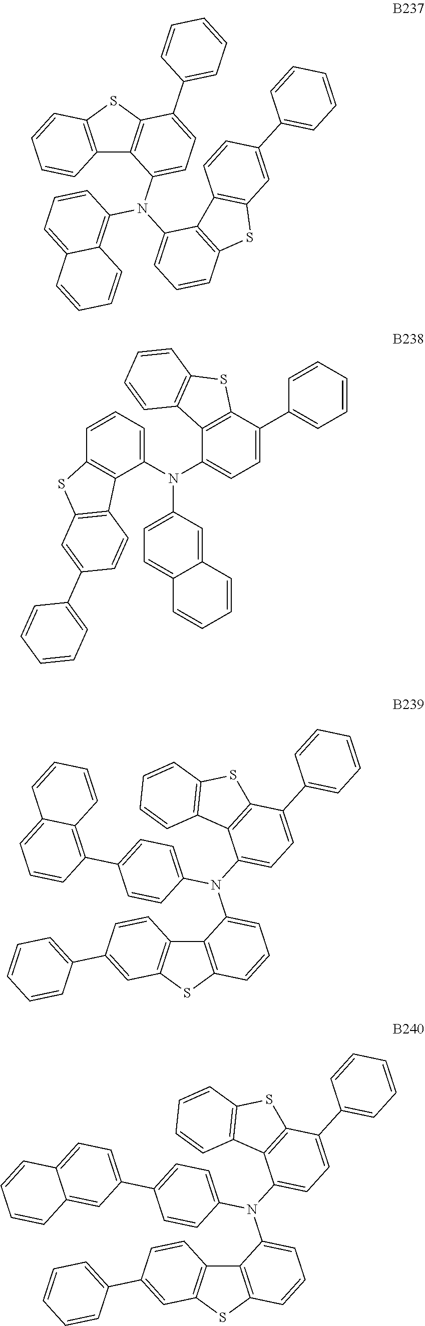 [SMILES: c1ccc(-c2ccc3c(c2)sc2cccc(N(c4cccc5ccccc45)c4ccc(-c5ccccc5)c5sc6ccccc6c45)c23)cc1 ; c1ccc(-c2ccc3c(c2)sc2cccc(N(c4ccc5ccccc5c4)c4ccc(-c5ccccc5)c5sc6ccccc6c45)c23)cc1 ; c1ccc(-c2ccc3c(c2)sc2cccc(N(c4ccc(-c5cccc6ccccc56)cc4)c4ccc(-c5ccccc5)c5sc6ccccc6c45)c23)cc1 ; c1ccc(-c2ccc3c(c2)sc2cccc(N(c4ccc(-c5ccc6ccccc6c5)cc4)c4ccc(-c5ccccc5)c5sc6ccccc6c45)c23)cc1]

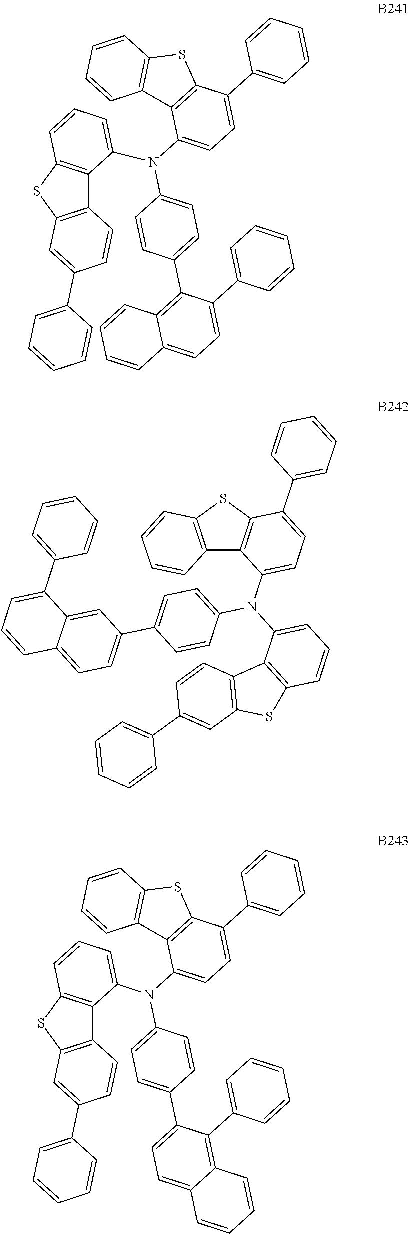 [SMILES: c1ccc(-c2ccc3c(c2)sc2cccc(N(c4ccc(-c5c(-c6ccccc6)ccc6ccccc56)cc4)c4ccc(-c5ccccc5)c5sc6ccccc6c45)c23)cc1 ; c1ccc(-c2ccc3c(c2)sc2cccc(N(c4ccc(-c5ccc6cccc(-c7ccccc7)c6c5)cc4)c4ccc(-c5ccccc5)c5sc6ccccc6c45)c23)cc1 ; c1ccc(-c2ccc3c(c2)sc2cccc(N(c4ccc(-c5ccc6ccccc6c5-c5ccccc5)cc4)c4ccc(-c5ccccc5)c5sc6ccccc6c45)c23)cc1]

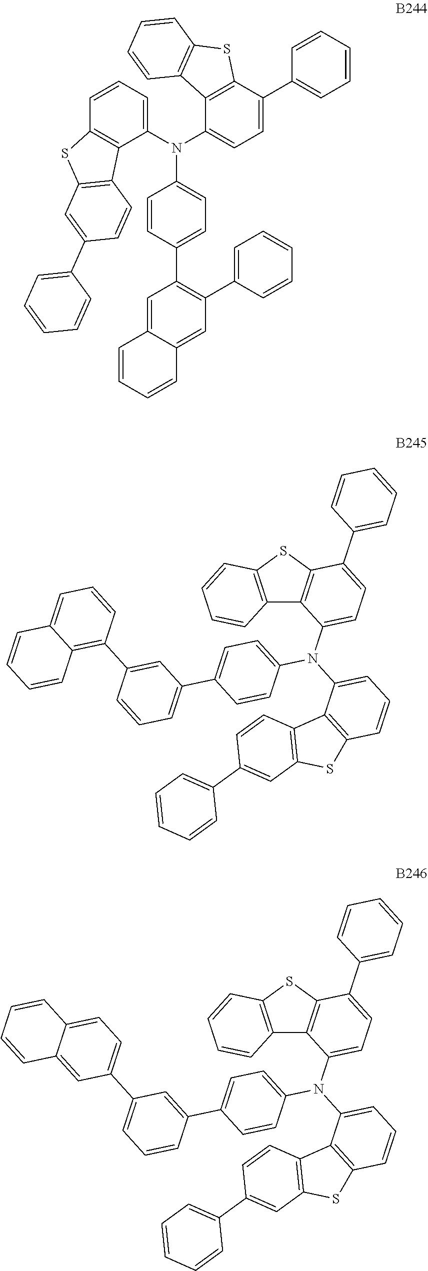 [SMILES: c1ccc(-c2ccc3c(c2)sc2cccc(N(c4ccc(-c5cc6ccccc6cc5-c5ccccc5)cc4)c4ccc(-c5ccccc5)c5sc6ccccc6c45)c23)cc1 ; c1ccc(-c2ccc3c(c2)sc2cccc(N(c4ccc(-c5cccc(-c6cccc7ccccc67)c5)cc4)c4ccc(-c5ccccc5)c5sc6ccccc6c45)c23)cc1 ; c1ccc(-c2ccc3c(c2)sc2cccc(N(c4ccc(-c5cccc(-c6ccc7ccccc7c6)c5)cc4)c4ccc(-c5ccccc5)c5sc6ccccc6c45)c23)cc1]

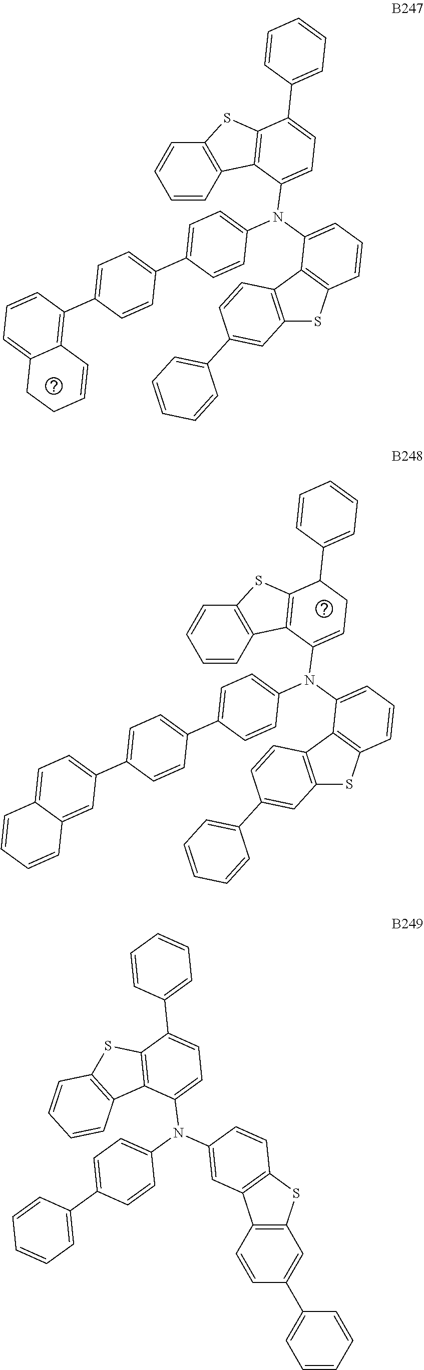 [SMILES: C1=Cc2c(cccc2-c2ccc(-c3ccc(N(c4cccc5sc6cc(-c7ccccc7)ccc6c45)c4ccc(-c5ccccc5)c5sc6ccccc6c45)cc3)cc2)CC1 ; C1=C(N(c2ccc(-c3ccc(-c4ccc5ccccc5c4)cc3)cc2)c2cccc3sc4cc(-c5ccccc5)ccc4c23)c2c(sc3ccccc23)C(c2ccccc2)C1 ; c1ccc(-c2ccc(N(c3ccc4sc5cc(-c6ccccc6)ccc5c4c3)c3ccc(-c4ccccc4)c4sc5ccccc5c34)cc2)cc1]

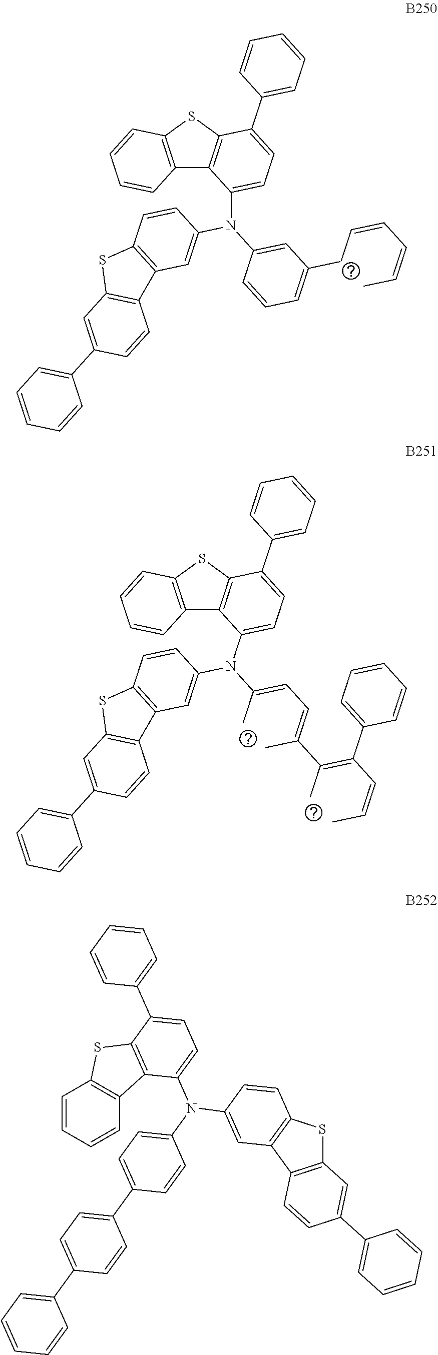 [SMILES: C/C=C\C=C/Cc1cccc(N(c2ccc3sc4cc(-c5ccccc5)ccc4c3c2)c2ccc(-c3ccccc3)c3sc4ccccc4c23)c1 ; C\C=C/C(=C(C)/C(C)=C/C=C(\C)N(c1ccc2sc3cc(-c4ccccc4)ccc3c2c1)c1ccc(-c2ccccc2)c2sc3ccccc3c12)c1ccccc1 ; c1ccc(-c2ccc(-c3ccc(N(c4ccc5sc6cc(-c7ccccc7)ccc6c5c4)c4ccc(-c5ccccc5)c5sc6ccccc6c45)cc3)cc2)cc1]

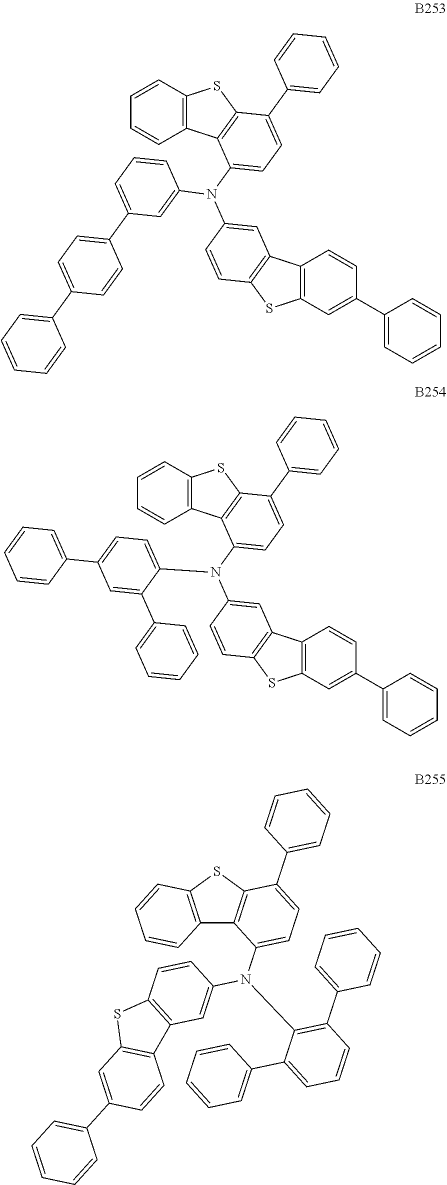 [SMILES: c1ccc(-c2ccc(-c3cccc(N(c4ccc5sc6cc(-c7ccccc7)ccc6c5c4)c4ccc(-c5ccccc5)c5sc6ccccc6c45)c3)cc2)cc1 ; c1ccc(-c2ccc(N(c3ccc4sc5cc(-c6ccccc6)ccc5c4c3)c3ccc(-c4ccccc4)c4sc5ccccc5c34)c(-c3ccccc3)c2)cc1 ; c1ccc(-c2ccc3c(c2)sc2ccc(N(c4c(-c5ccccc5)cccc4-c4ccccc4)c4ccc(-c5ccccc5)c5sc6ccccc6c45)cc23)cc1]

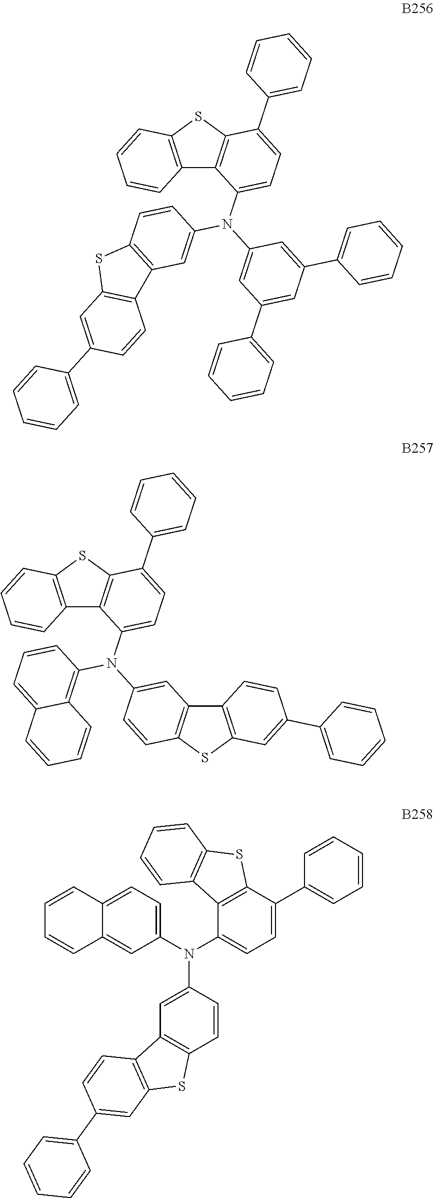 [SMILES: c1ccc(-c2cc(-c3ccccc3)cc(N(c3ccc4sc5cc(-c6ccccc6)ccc5c4c3)c3ccc(-c4ccccc4)c4sc5ccccc5c34)c2)cc1 ; c1ccc(-c2ccc3c(c2)sc2ccc(N(c4cccc5ccccc45)c4ccc(-c5ccccc5)c5sc6ccccc6c45)cc23)cc1 ; c1ccc(-c2ccc3c(c2)sc2ccc(N(c4ccc5ccccc5c4)c4ccc(-c5ccccc5)c5sc6ccccc6c45)cc23)cc1]

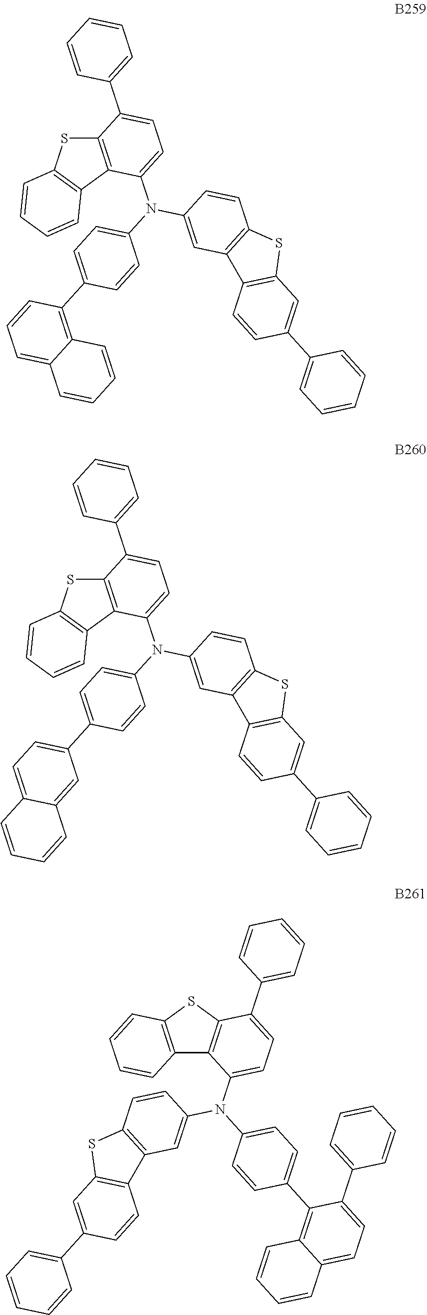 [SMILES: c1ccc(-c2ccc3c(c2)sc2ccc(N(c4ccc(-c5cccc6ccccc56)cc4)c4ccc(-c5ccccc5)c5sc6ccccc6c45)cc23)cc1 ; c1ccc(-c2ccc3c(c2)sc2ccc(N(c4ccc(-c5ccc6ccccc6c5)cc4)c4ccc(-c5ccccc5)c5sc6ccccc6c45)cc23)cc1 ; c1ccc(-c2ccc3c(c2)sc2ccc(N(c4ccc(-c5c(-c6ccccc6)ccc6ccccc56)cc4)c4ccc(-c5ccccc5)c5sc6ccccc6c45)cc23)cc1]

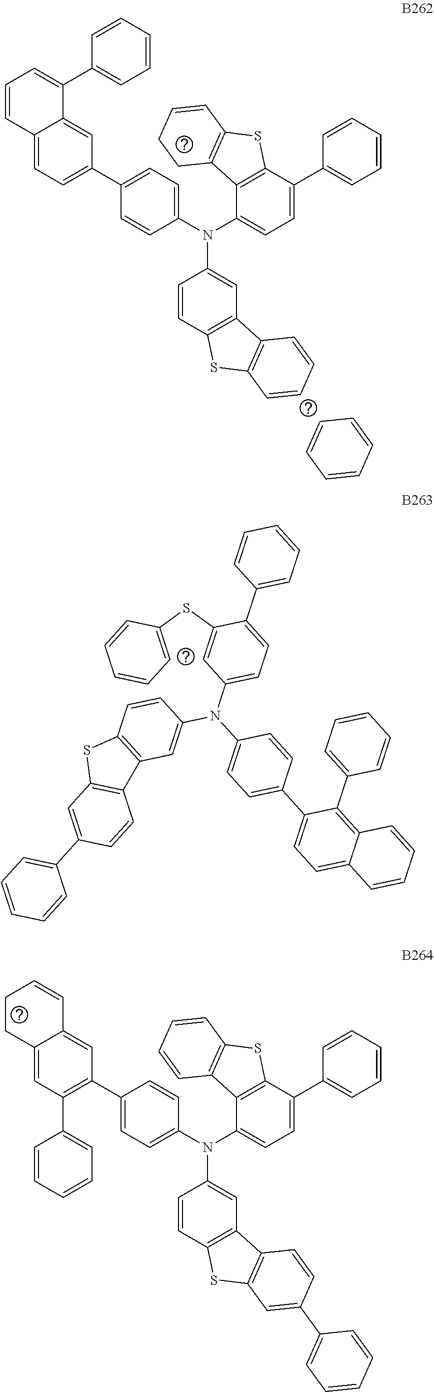

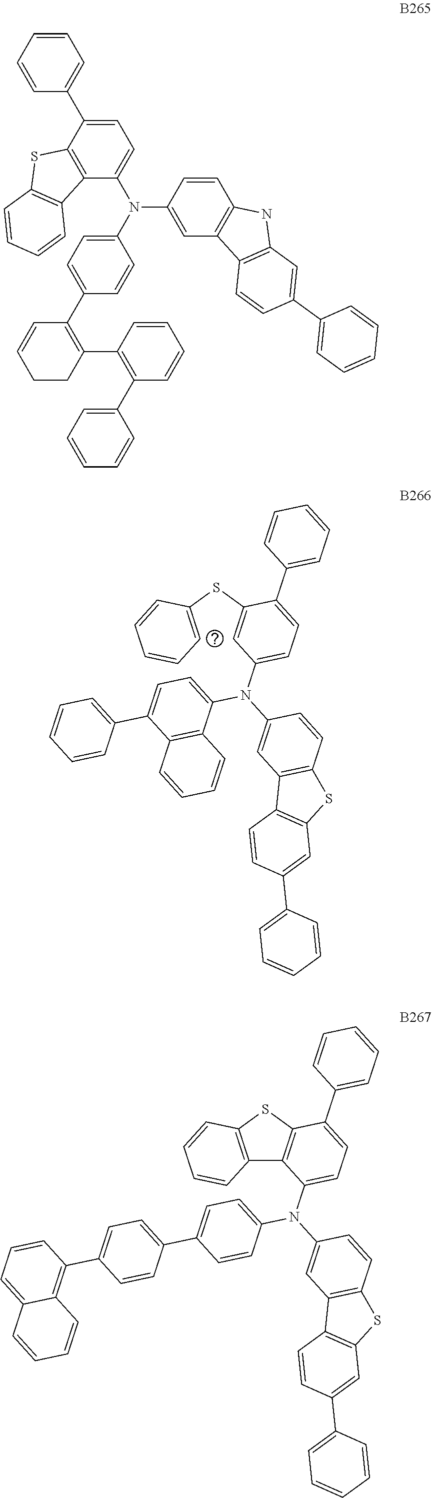 [SMILES: C1=CC(c2ccc(N(c3ccc4[nH]c5cc(-c6ccccc6)ccc5c4c3)c3ccc(-c4ccccc4)c4sc5ccccc5c34)cc2)=C(c2ccccc2-c2ccccc2)CC1 ; c1ccc(Sc2cc(N(c3ccc4sc5cc(-c6ccccc6)ccc5c4c3)c3ccc(-c4ccccc4)c4ccccc34)ccc2-c2ccccc2)cc1 ; c1ccc(-c2ccc3c(c2)sc2ccc(N(c4ccc(-c5ccc(-c6cccc7ccccc67)cc5)cc4)c4ccc(-c5ccccc5)c5sc6ccccc6c45)cc23)cc1]

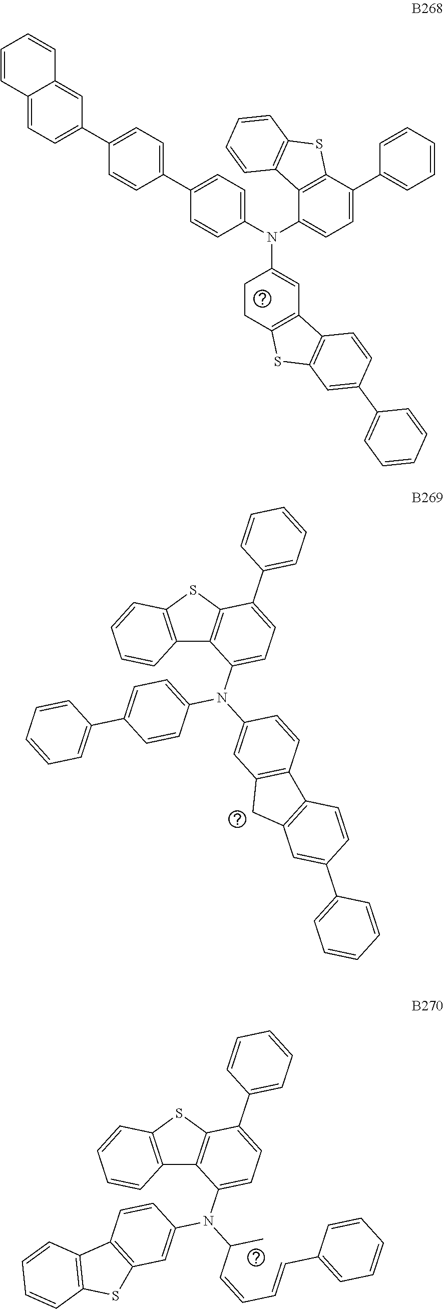 [SMILES: C1=C(N(c2ccc(-c3ccc(-c4ccc5ccccc5c4)cc3)cc2)c2ccc(-c3ccccc3)c3sc4ccccc4c23)CCc2sc3cc(-c4ccccc4)ccc3c21 ; c1ccc(-c2ccc(N(c3ccc4c(c3)Cc3cc(-c5ccccc5)ccc3-4)c3ccc(-c4ccccc4)c4sc5ccccc5c34)cc2)cc1 ; CC(/C=C\C=C\c1ccccc1)N(c1ccc2c(c1)sc1ccccc12)c1ccc(-c2ccccc2)c2sc3ccccc3c12]

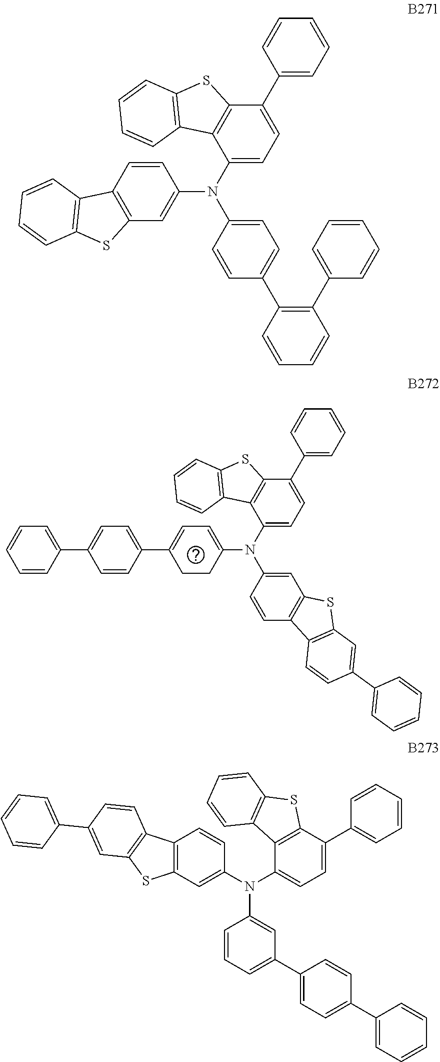 [SMILES: c1ccc(-c2ccccc2-c2ccc(N(c3ccc4c(c3)sc3ccccc34)c3ccc(-c4ccccc4)c4sc5ccccc5c34)cc2)cc1 ; C1=C(c2ccc(-c3ccccc3)cc2)CCC(N(c2ccc3c(c2)sc2cc(-c4ccccc4)ccc23)c2ccc(-c3ccccc3)c3sc4ccccc4c23)=C1 ; c1ccc(-c2ccc(-c3cccc(N(c4ccc5c(c4)sc4cc(-c6ccccc6)ccc45)c4ccc(-c5ccccc5)c5sc6ccccc6c45)c3)cc2)cc1]

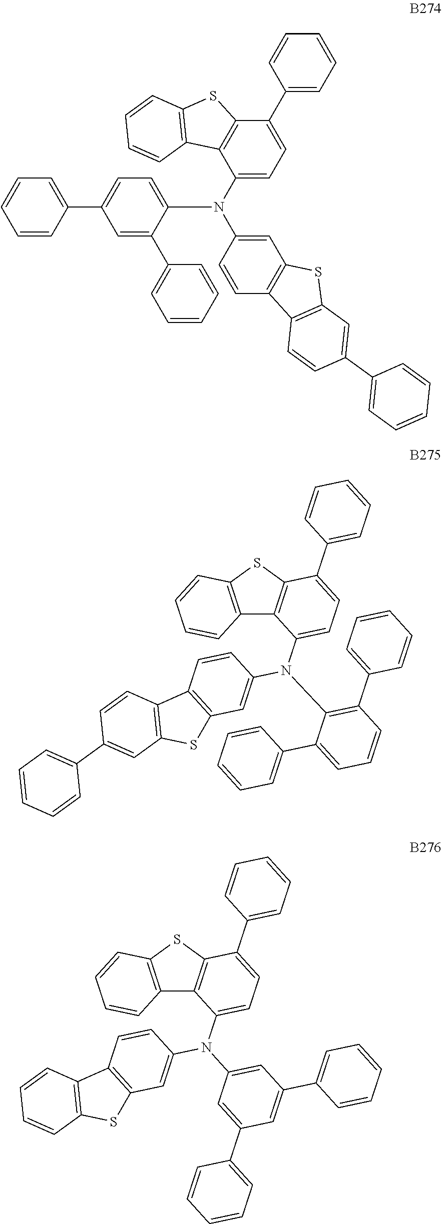 [SMILES: c1ccc(-c2ccc(N(c3ccc4c(c3)sc3cc(-c5ccccc5)ccc34)c3ccc(-c4ccccc4)c4sc5ccccc5c34)c(-c3ccccc3)c2)cc1 ; c1ccc(-c2ccc3c(c2)sc2cc(N(c4c(-c5ccccc5)cccc4-c4ccccc4)c4ccc(-c5ccccc5)c5sc6ccccc6c45)ccc23)cc1 ; c1ccc(-c2cc(-c3ccccc3)cc(N(c3ccc4c(c3)sc3ccccc34)c3ccc(-c4ccccc4)c4sc5ccccc5c34)c2)cc1]

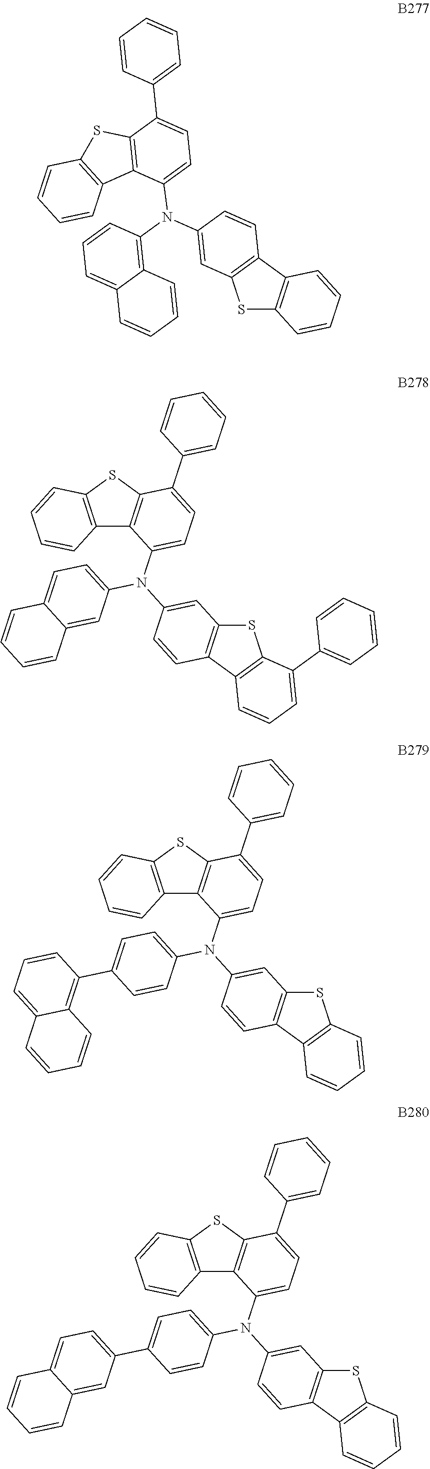

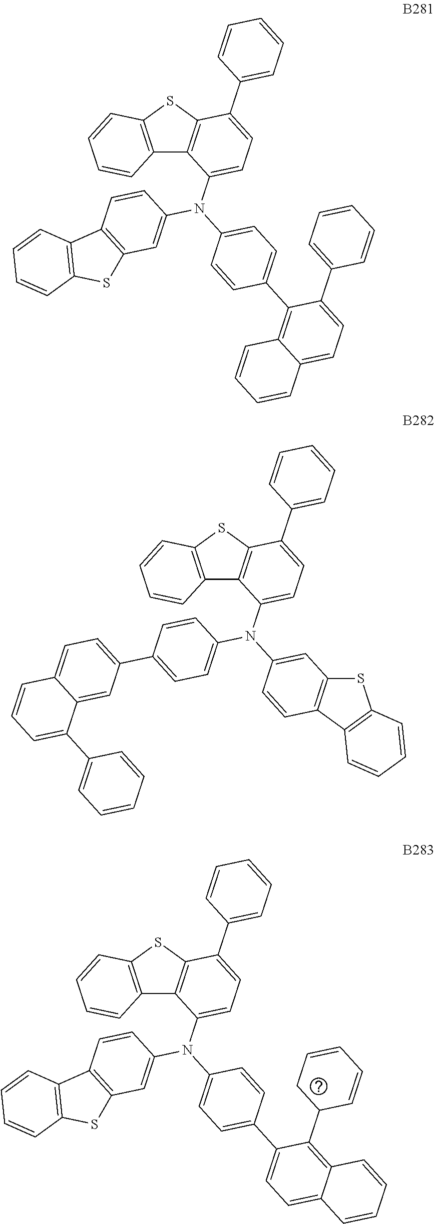 [SMILES: c1ccc(-c2ccc3ccccc3c2-c2ccc(N(c3ccc4c(c3)sc3ccccc34)c3ccc(-c4ccccc4)c4sc5ccccc5c34)cc2)cc1 ; c1ccc(-c2cccc3ccc(-c4ccc(N(c5ccc6c(c5)sc5ccccc56)c5ccc(-c6ccccc6)c6sc7ccccc7c56)cc4)cc23)cc1 ; C1=CCC(c2c(-c3ccc(N(c4ccc5c(c4)sc4ccccc45)c4ccc(-c5ccccc5)c5sc6ccccc6c45)cc3)ccc3ccccc23)C=C1]

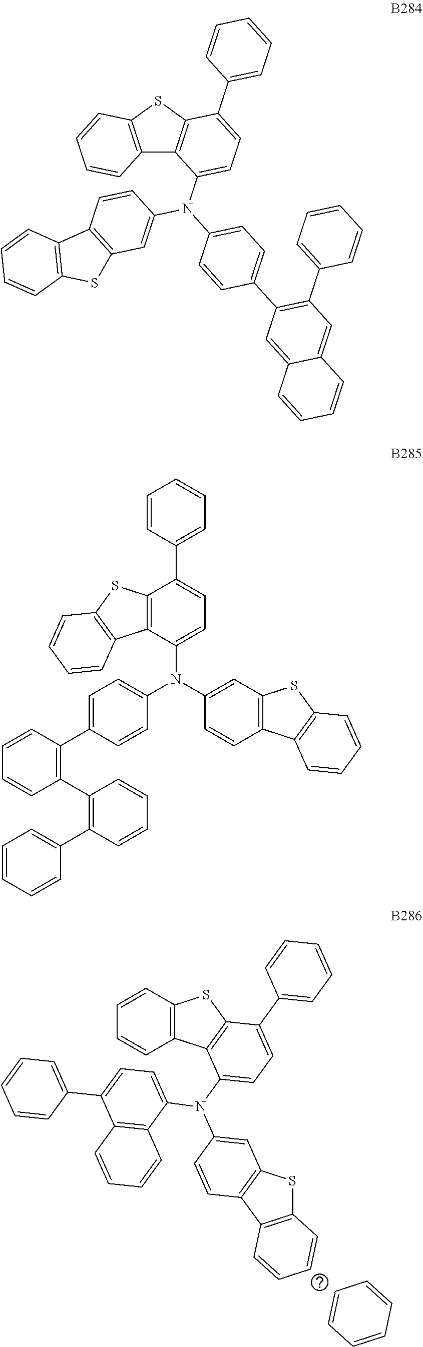

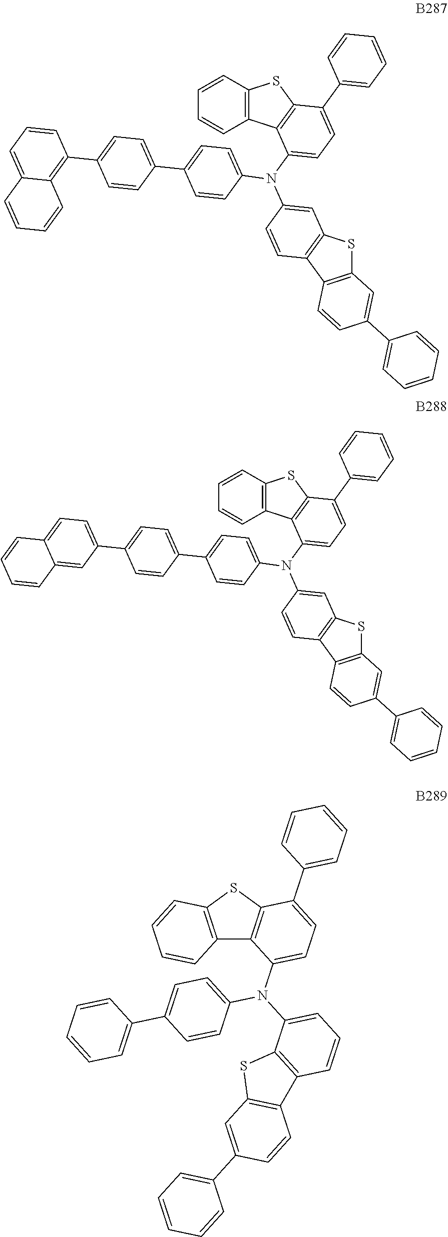 [SMILES: c1ccc(-c2ccc3c(c2)sc2cc(N(c4ccc(-c5ccc(-c6cccc7ccccc67)cc5)cc4)c4ccc(-c5ccccc5)c5sc6ccccc6c45)ccc23)cc1 ; c1ccc(-c2ccc3c(c2)sc2cc(N(c4ccc(-c5ccc(-c6ccc7ccccc7c6)cc5)cc4)c4ccc(-c5ccccc5)c5sc6ccccc6c45)ccc23)cc1 ; c1ccc(-c2ccc(N(c3cccc4c3sc3cc(-c5ccccc5)ccc34)c3ccc(-c4ccccc4)c4sc5ccccc5c34)cc2)cc1]

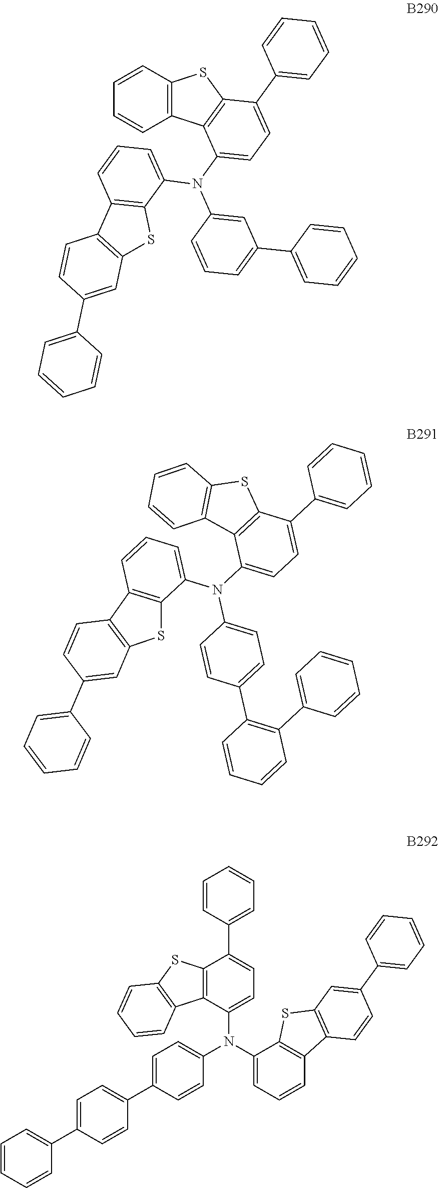 [SMILES: c1ccc(-c2cccc(N(c3cccc4c3sc3cc(-c5ccccc5)ccc34)c3ccc(-c4ccccc4)c4sc5ccccc5c34)c2)cc1 ; c1ccc(-c2ccc3c(c2)sc2c(N(c4ccc(-c5ccccc5-c5ccccc5)cc4)c4ccc(-c5ccccc5)c5sc6ccccc6c45)cccc23)cc1 ; c1ccc(-c2ccc(-c3ccc(N(c4cccc5c4sc4cc(-c6ccccc6)ccc45)c4ccc(-c5ccccc5)c5sc6ccccc6c45)cc3)cc2)cc1]

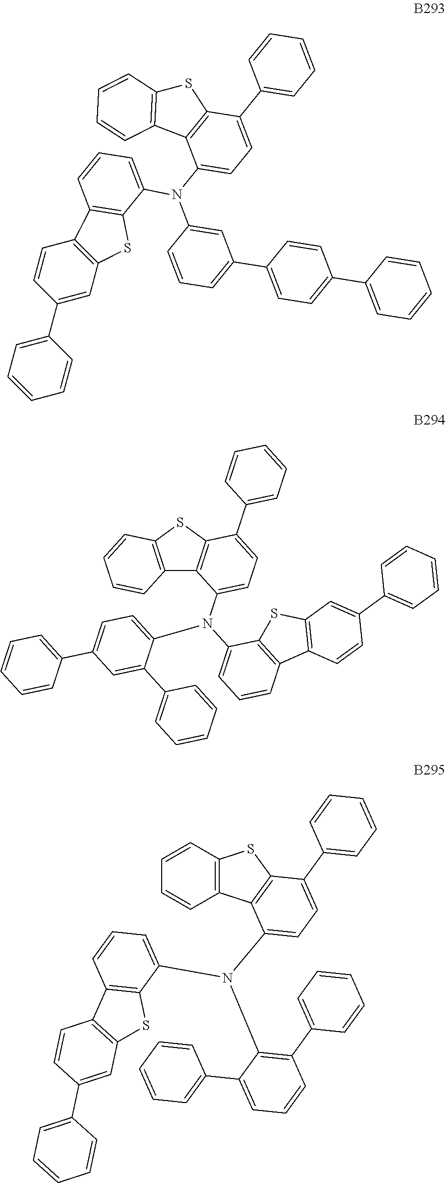 [SMILES: c1ccc(-c2ccc(-c3cccc(N(c4cccc5c4sc4cc(-c6ccccc6)ccc45)c4ccc(-c5ccccc5)c5sc6ccccc6c45)c3)cc2)cc1 ; c1ccc(-c2ccc(N(c3cccc4c3sc3cc(-c5ccccc5)ccc34)c3ccc(-c4ccccc4)c4sc5ccccc5c34)c(-c3ccccc3)c2)cc1 ; c1ccc(-c2ccc3c(c2)sc2c(N(c4c(-c5ccccc5)cccc4-c4ccccc4)c4ccc(-c5ccccc5)c5sc6ccccc6c45)cccc23)cc1]

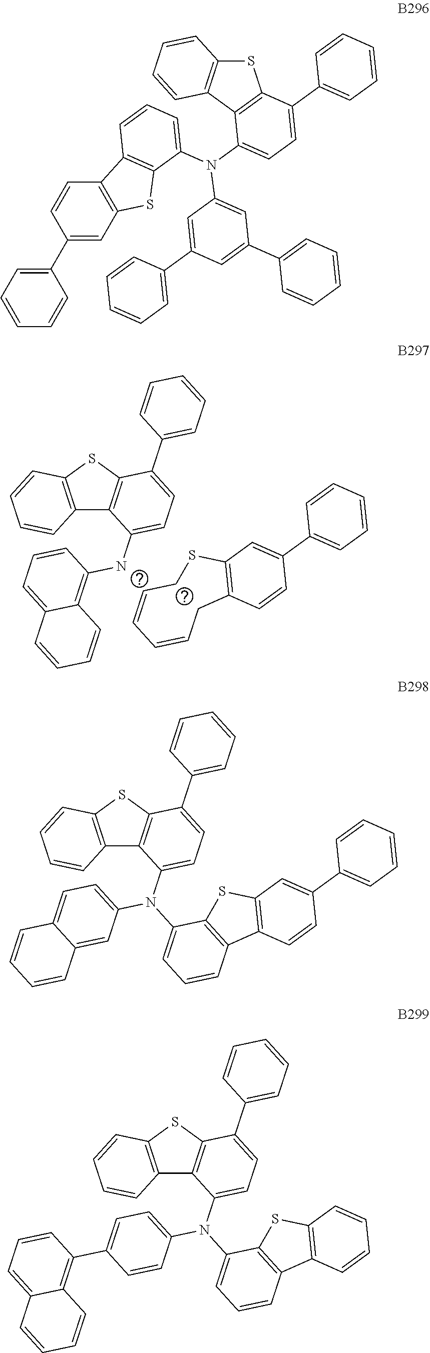

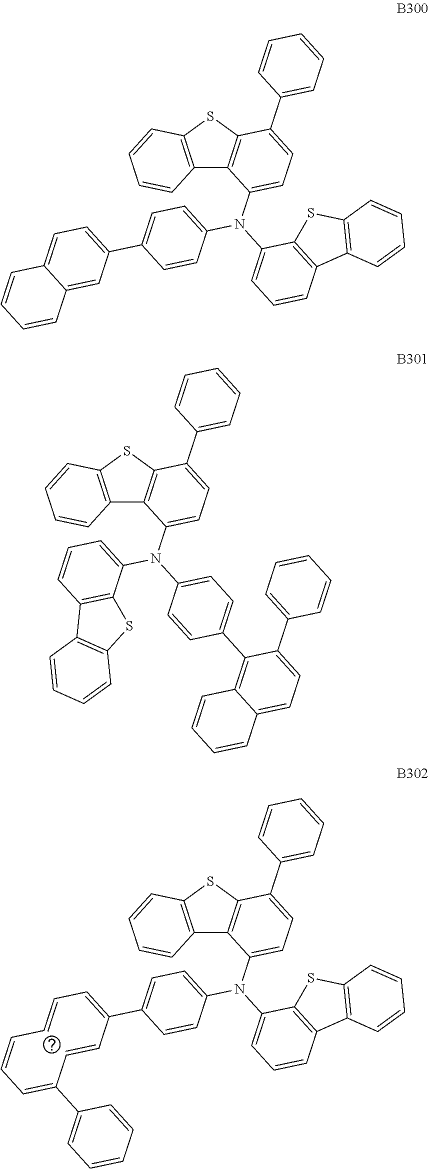 [SMILES: c1ccc(-c2ccc(N(c3ccc(-c4ccc5ccccc5c4)cc3)c3cccc4c3sc3ccccc34)c3c2sc2ccccc23)cc1 ; c1ccc(-c2ccc3ccccc3c2-c2ccc(N(c3cccc4c3sc3ccccc34)c3ccc(-c4ccccc4)c4sc5ccccc5c34)cc2)cc1 ; c1ccc(-c2ccccccc(-c3ccc(N(c4cccc5c4sc4ccccc45)c4ccc(-c5ccccc5)c5sc6ccccc6c45)cc3)cc2)cc1]

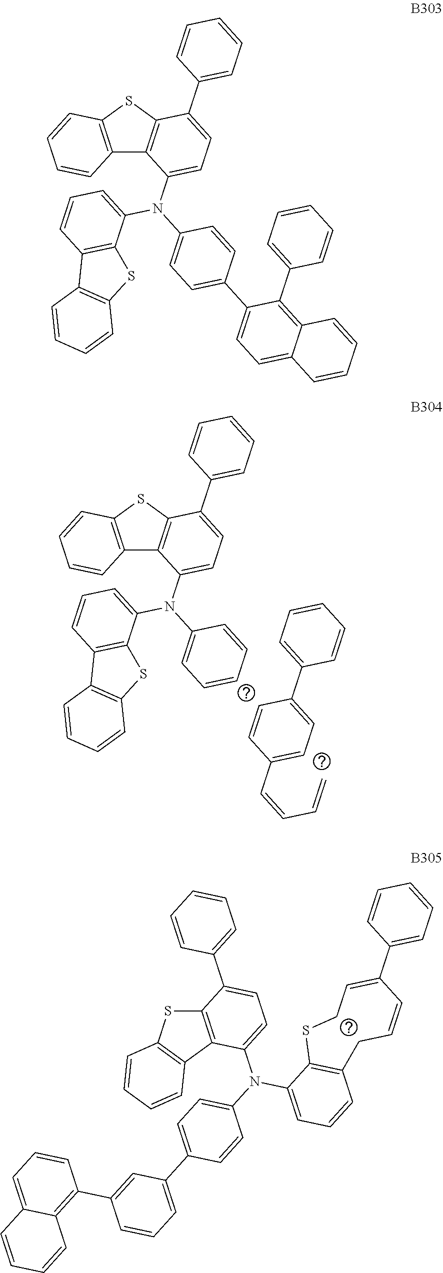

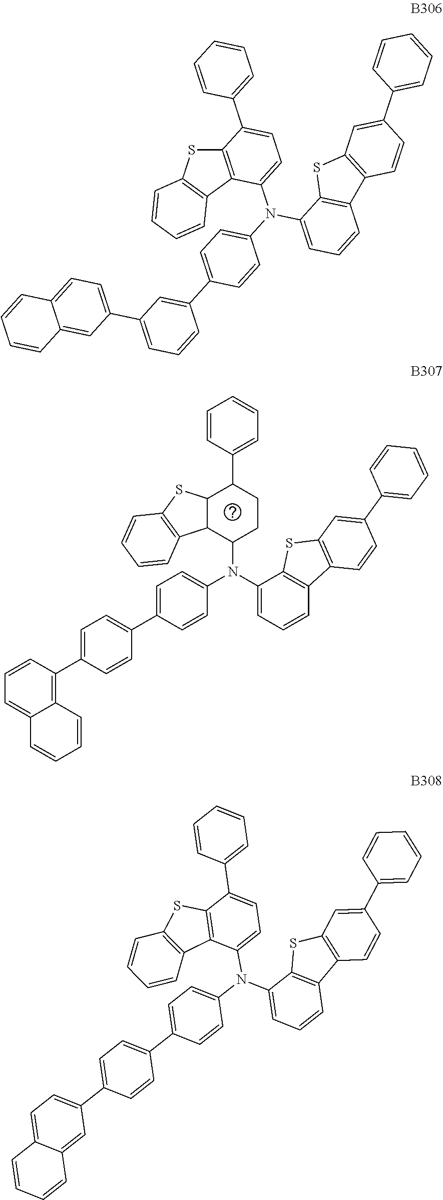 [SMILES: c1ccc(-c2ccc3c(c2)sc2c(N(c4ccc(-c5cccc(-c6ccc7ccccc7c6)c5)cc4)c4ccc(-c5ccccc5)c5sc6ccccc6c45)cccc23)cc1 ; c1ccc(-c2ccc3c(c2)sc2c(N(c4ccc(-c5ccc(-c6cccc7ccccc67)cc5)cc4)C4CCC(c5ccccc5)C5Sc6ccccc6C54)cccc23)cc1 ; c1ccc(-c2ccc3c(c2)sc2c(N(c4ccc(-c5ccc(-c6ccc7ccccc7c6)cc5)cc4)c4ccc(-c5ccccc5)c5sc6ccccc6c45)cccc23)cc1]

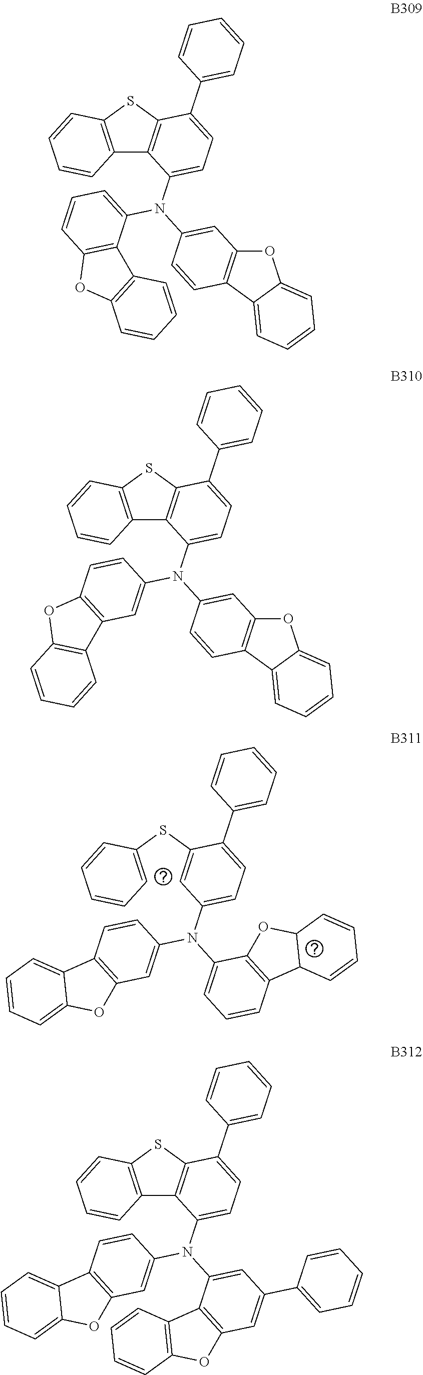 [SMILES: c1ccc(-c2ccc(N(c3ccc4c(c3)oc3ccccc34)c3cccc4oc5ccccc5c34)c3c2sc2ccccc23)cc1 ; c1ccc(-c2ccc(N(c3ccc4c(c3)oc3ccccc34)c3ccc4oc5ccccc5c4c3)c3c2sc2ccccc23)cc1 ; C1=CC2Oc3c(cccc3N(c3ccc(-c4ccccc4)c(Sc4ccccc4)c3)c3ccc4c(c3)oc3ccccc34)C2C=C1 ; c1ccc(-c2cc(N(c3ccc4c(c3)oc3ccccc34)c3ccc(-c4ccccc4)c4sc5ccccc5c34)c3c(c2)oc2ccccc23)cc1]

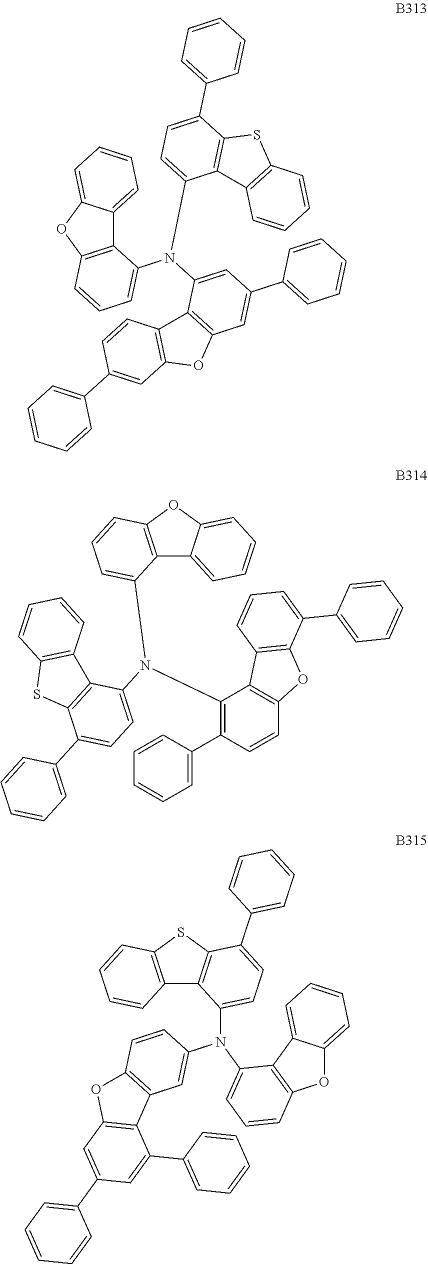 [SMILES: c1ccc(-c2ccc3c(c2)oc2cc(-c4ccccc4)cc(N(c4cccc5oc6ccccc6c45)c4ccc(-c5ccccc5)c5sc6ccccc6c45)c23)cc1 ; c1ccc(-c2ccc3oc4c(-c5ccccc5)cccc4c3c2N(c2cccc3oc4ccccc4c23)c2ccc(-c3ccccc3)c3sc4ccccc4c23)cc1 ; c1ccc(-c2cc(-c3ccccc3)c3c(c2)oc2ccc(N(c4cccc5oc6ccccc6c45)c4ccc(-c5ccccc5)c5sc6ccccc6c45)cc23)cc1]

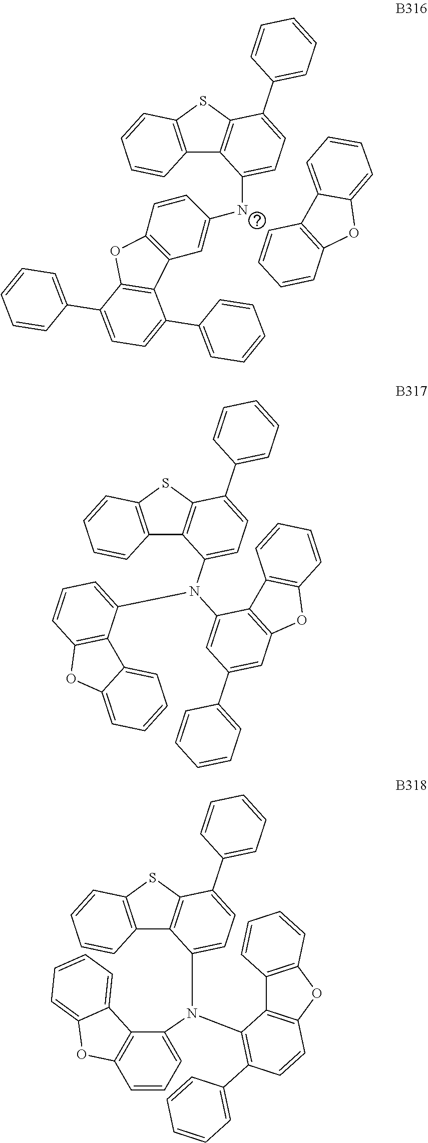

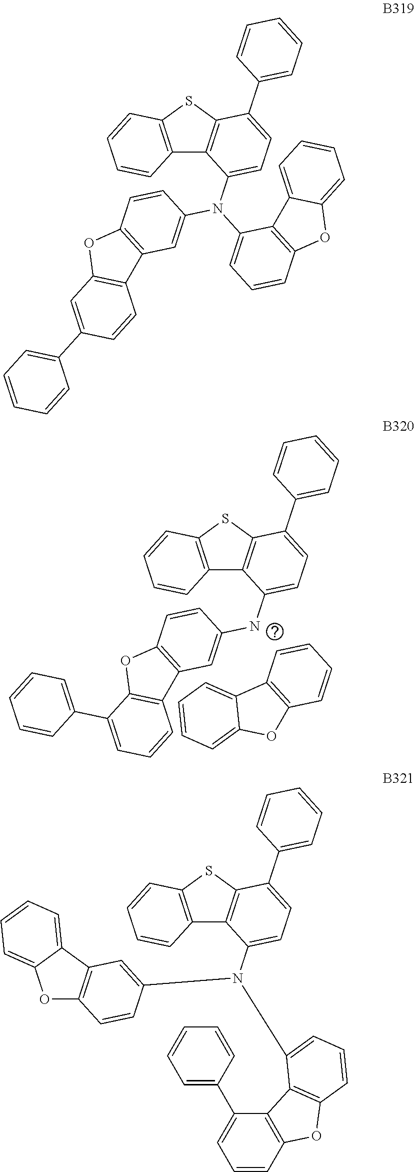

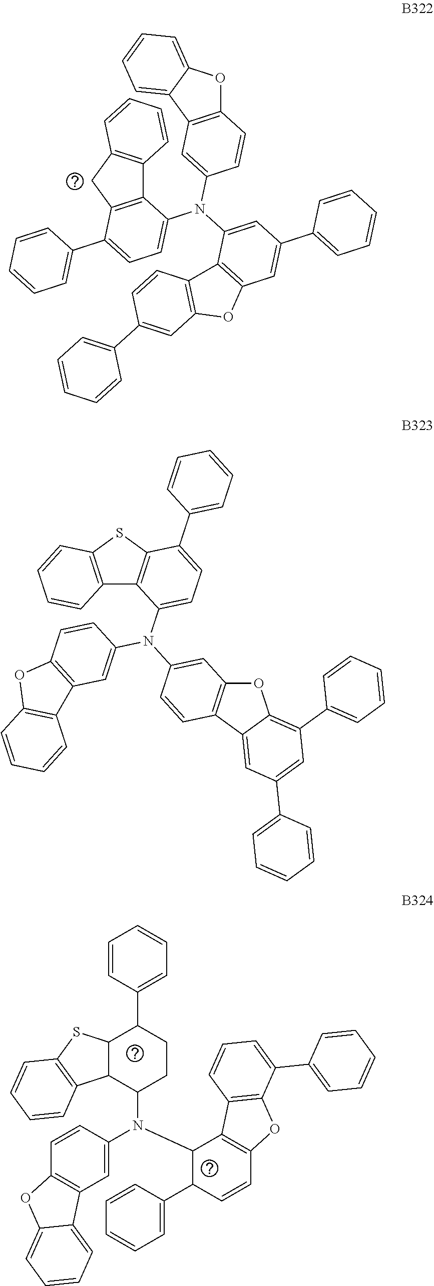 [SMILES: c1ccc(-c2ccc3c(c2)oc2cc(-c4ccccc4)cc(N(c4ccc5oc6ccccc6c5c4)c4ccc(-c5ccccc5)c5c4-c4ccccc4C5)c23)cc1 ; c1ccc(-c2cc(-c3ccccc3)c3oc4cc(N(c5ccc6oc7ccccc7c6c5)c5ccc(-c6ccccc6)c6sc7ccccc7c56)ccc4c3c2)cc1 ; C1=CC(c2ccccc2)C(N(c2ccc3oc4ccccc4c3c2)C2CCC(c3ccccc3)C3Sc4ccccc4C32)c2c1oc1c(-c3ccccc3)cccc21]

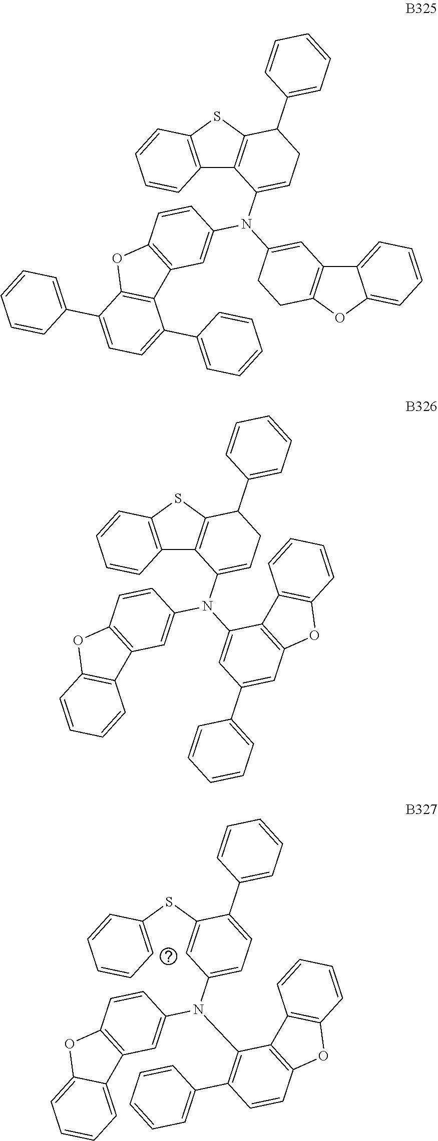 [SMILES: C1=C(N(C2=CCC(c3ccccc3)c3sc4ccccc4c32)c2ccc3oc4c(-c5ccccc5)ccc(-c5ccccc5)c4c3c2)CCc2oc3ccccc3c21 ; C1=C(N(c2ccc3oc4ccccc4c3c2)c2cc(-c3ccccc3)cc3oc4ccccc4c23)c2c(sc3ccccc23)C(c2ccccc2)C1 ; c1ccc(Sc2cc(N(c3ccc4oc5ccccc5c4c3)c3c(-c4ccccc4)ccc4oc5ccccc5c34)ccc2-c2ccccc2)cc1]

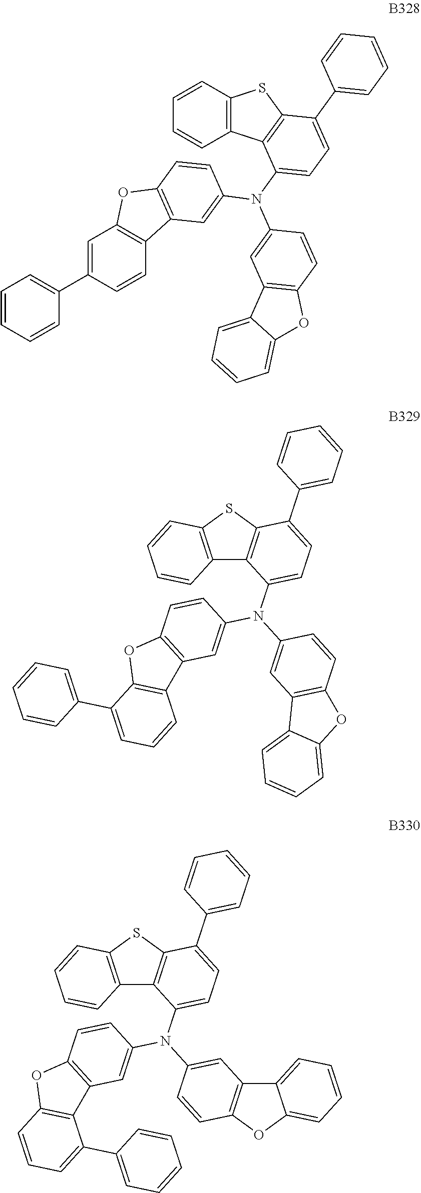 [SMILES: c1ccc(-c2ccc3c(c2)oc2ccc(N(c4ccc5oc6ccccc6c5c4)c4ccc(-c5ccccc5)c5sc6ccccc6c45)cc23)cc1 ; c1ccc(-c2cccc3c2oc2ccc(N(c4ccc5oc6ccccc6c5c4)c4ccc(-c5ccccc5)c5sc6ccccc6c45)cc23)cc1 ; c1ccc(-c2ccc(N(c3ccc4oc5ccccc5c4c3)c3ccc4oc5cccc(-c6ccccc6)c5c4c3)c3c2sc2ccccc23)cc1]

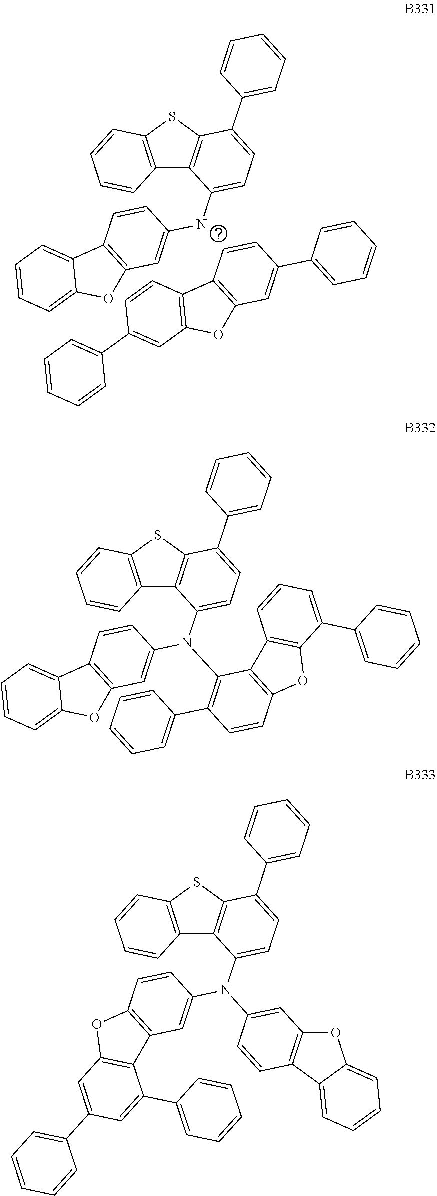

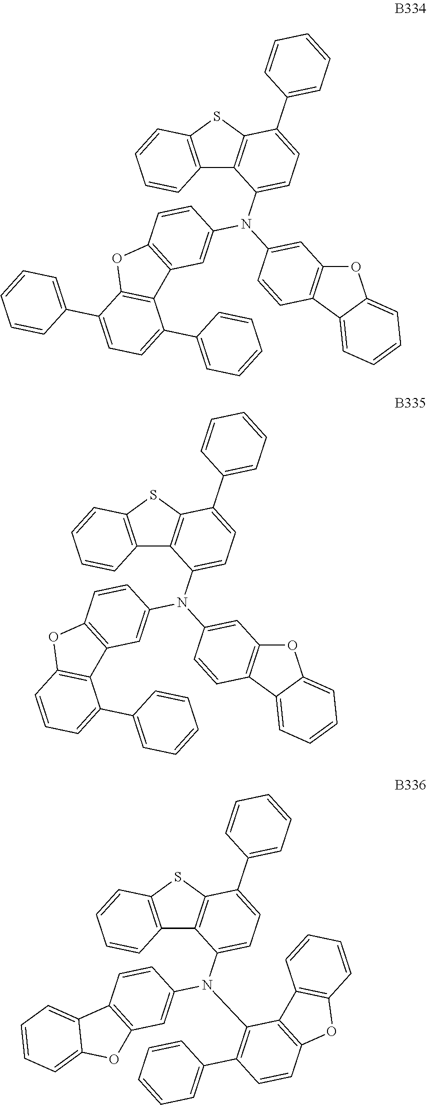

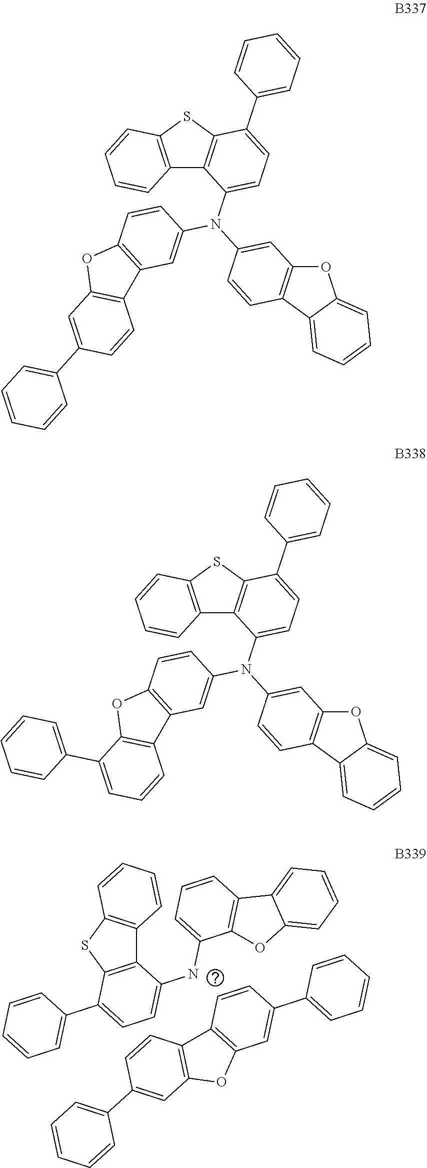

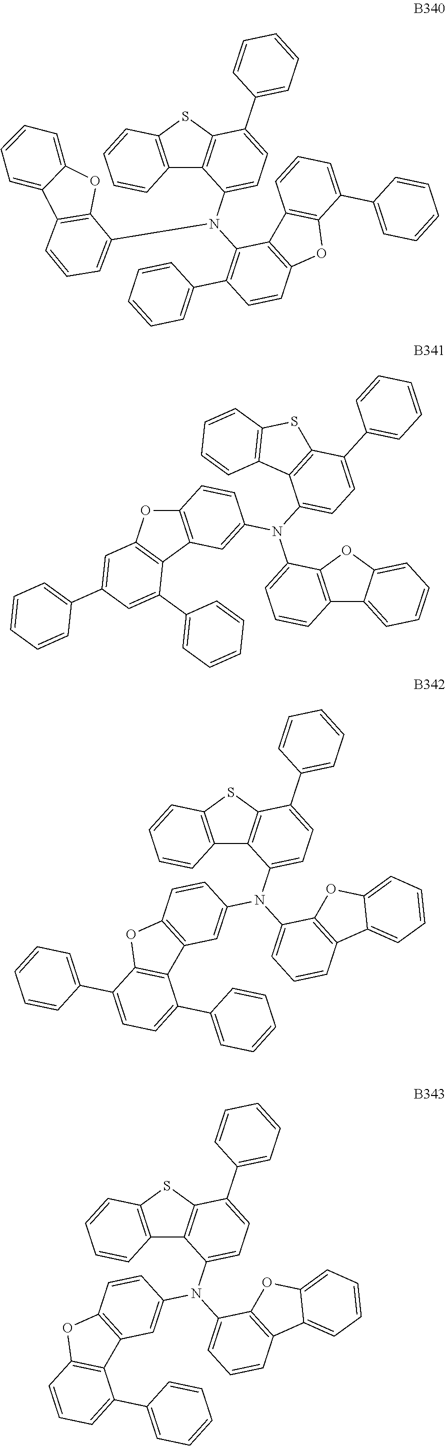 [SMILES: c1ccc(-c2ccc3oc4c(-c5ccccc5)cccc4c3c2N(c2cccc3c2oc2ccccc23)c2ccc(-c3ccccc3)c3sc4ccccc4c23)cc1 ; c1ccc(-c2cc(-c3ccccc3)c3c(c2)oc2ccc(N(c4cccc5c4oc4ccccc45)c4ccc(-c5ccccc5)c5sc6ccccc6c45)cc23)cc1 ; c1ccc(-c2ccc(-c3ccccc3)c3c2oc2ccc(N(c4cccc5c4oc4ccccc45)c4ccc(-c5ccccc5)c5sc6ccccc6c45)cc23)cc1 ; c1ccc(-c2ccc(N(c3ccc4oc5cccc(-c6ccccc6)c5c4c3)c3cccc4c3oc3ccccc34)c3c2sc2ccccc23)cc1]

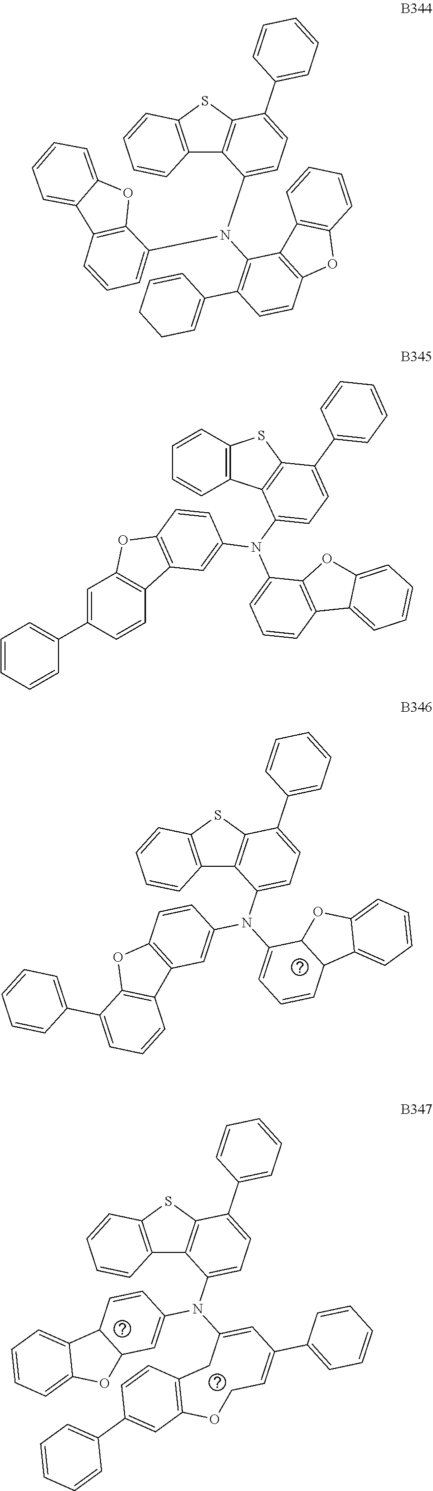 [SMILES: C1=CC(c2ccc3oc4ccccc4c3c2N(c2cccc3c2oc2ccccc23)c2ccc(-c3ccccc3)c3sc4ccccc4c23)=CCC1 ; c1ccc(-c2ccc3c(c2)oc2ccc(N(c4cccc5c4oc4ccccc45)c4ccc(-c5ccccc5)c5sc6ccccc6c45)cc23)cc1 ; C1=CC2c3ccccc3OC2C(N(c2ccc3oc4c(-c5ccccc5)cccc4c3c2)c2ccc(-c3ccccc3)c3sc4ccccc4c23)=C1 ; C1=CC2c3ccccc3OC2C=C1N(/C1=C/C(c2ccccc2)=C\COc2cc(-c3ccccc3)ccc2C1)c1ccc(-c2ccccc2)c2sc3ccccc3c12]

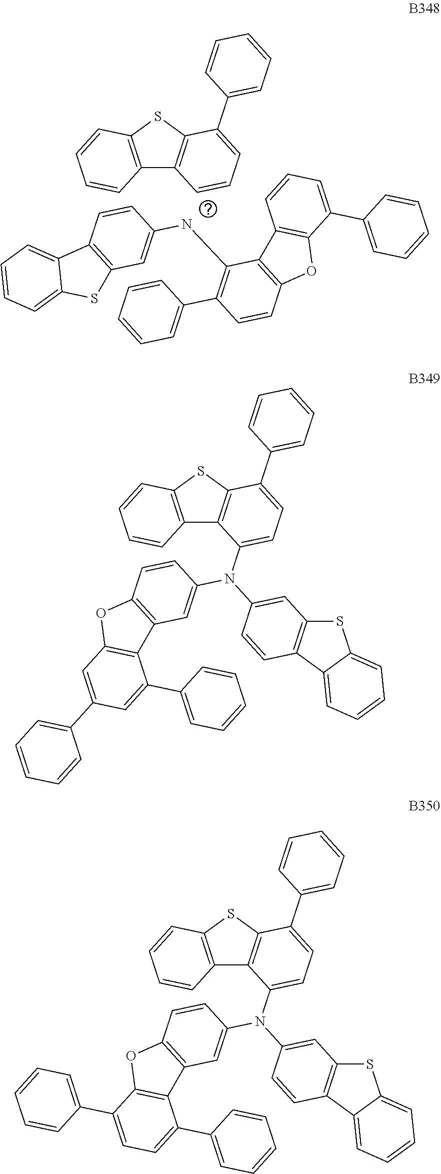

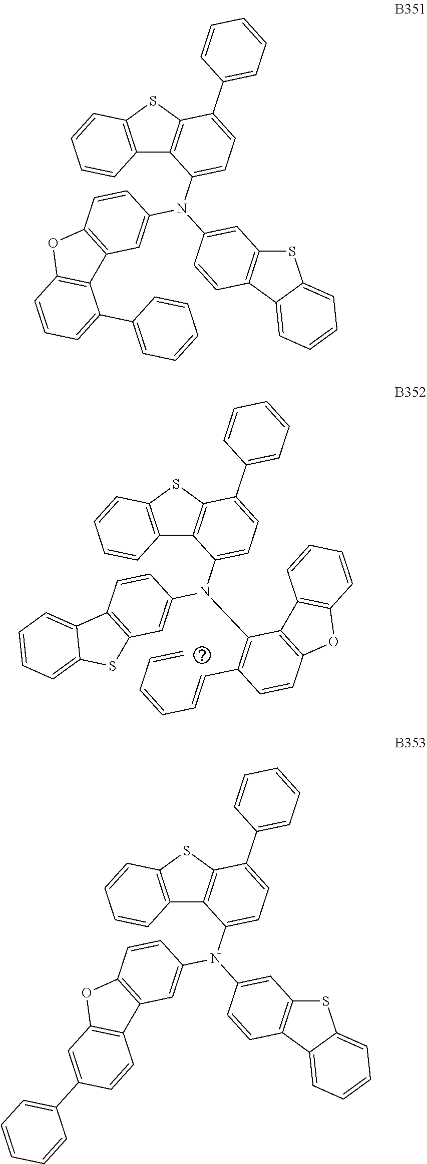 [SMILES: c1ccc(-c2ccc(N(c3ccc4c(c3)sc3ccccc34)c3ccc4oc5cccc(-c6ccccc6)c5c4c3)c3c2sc2ccccc23)cc1 ; C=C/C=C\C=C\c1ccc2oc3ccccc3c2c1N(c1ccc2c(c1)sc1ccccc12)c1ccc(-c2ccccc2)c2sc3ccccc3c12 ; c1ccc(-c2ccc3c(c2)oc2ccc(N(c4ccc5c(c4)sc4ccccc45)c4ccc(-c5ccccc5)c5sc6ccccc6c45)cc23)cc1]

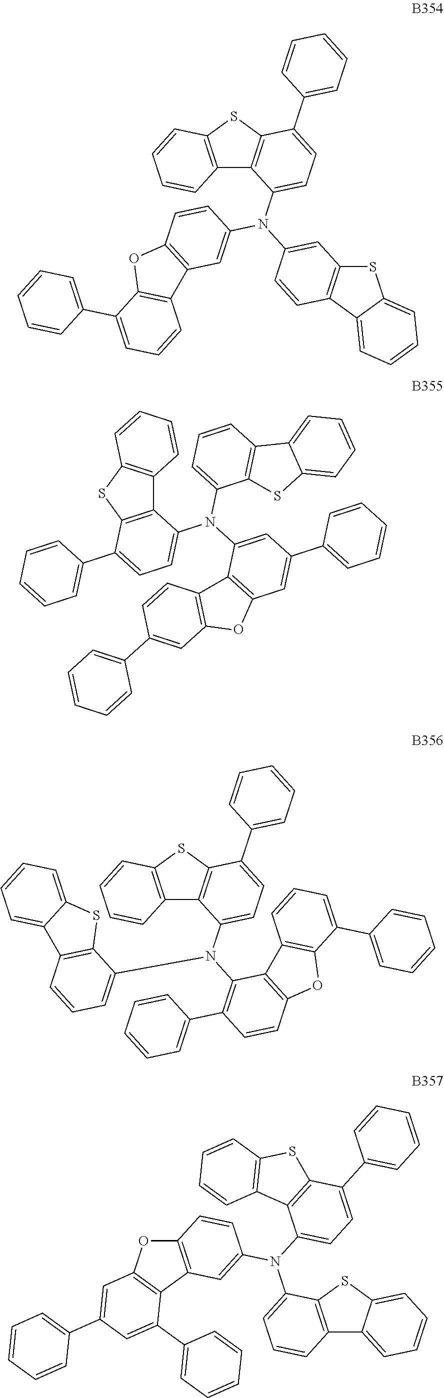 [SMILES: c1ccc(-c2cccc3c2oc2ccc(N(c4ccc5c(c4)sc4ccccc45)c4ccc(-c5ccccc5)c5sc6ccccc6c45)cc23)cc1 ; c1ccc(-c2ccc3c(c2)oc2cc(-c4ccccc4)cc(N(c4cccc5c4sc4ccccc45)c4ccc(-c5ccccc5)c5sc6ccccc6c45)c23)cc1 ; c1ccc(-c2ccc3oc4c(-c5ccccc5)cccc4c3c2N(c2cccc3c2sc2ccccc23)c2ccc(-c3ccccc3)c3sc4ccccc4c23)cc1 ; c1ccc(-c2cc(-c3ccccc3)c3c(c2)oc2ccc(N(c4cccc5c4sc4ccccc45)c4ccc(-c5ccccc5)c5sc6ccccc6c45)cc23)cc1]

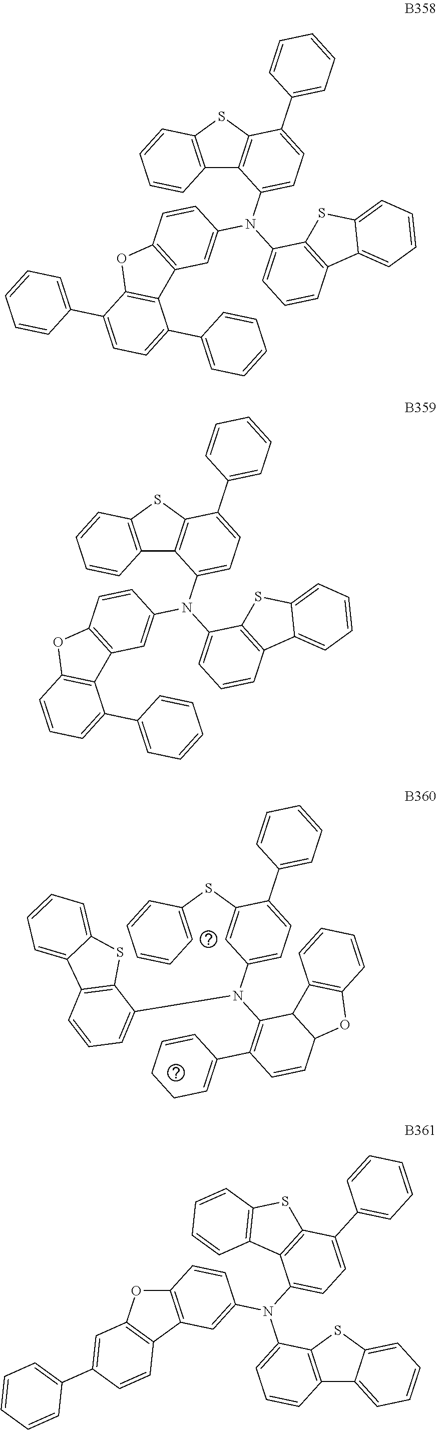

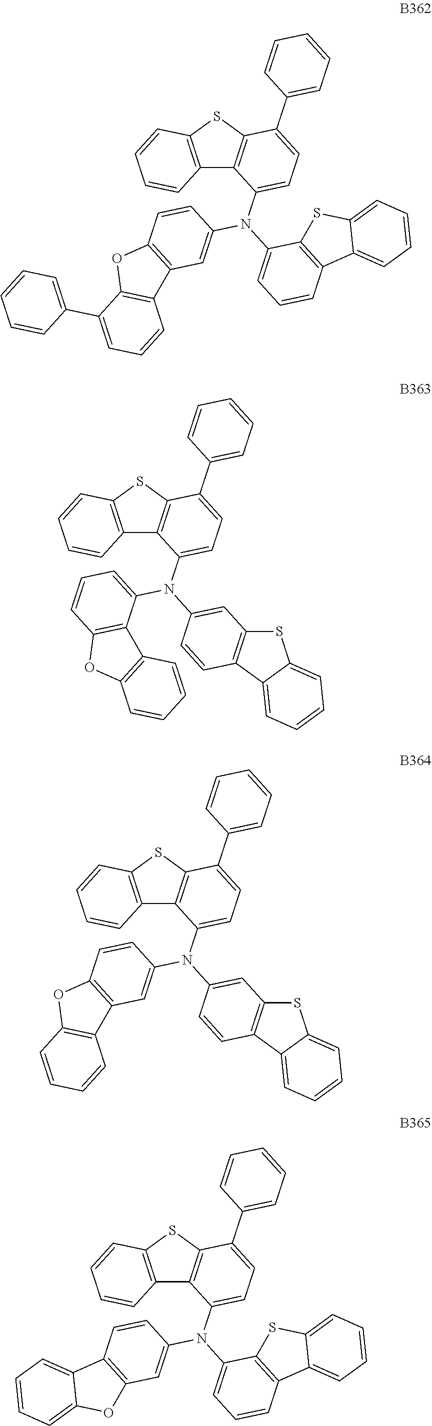 [SMILES: c1ccc(-c2cccc3c2oc2ccc(N(c4cccc5c4sc4ccccc45)c4ccc(-c5ccccc5)c5sc6ccccc6c45)cc23)cc1 ; c1ccc(-c2ccc(N(c3ccc4c(c3)sc3ccccc34)c3cccc4oc5ccccc5c34)c3c2sc2ccccc23)cc1 ; c1ccc(-c2ccc(N(c3ccc4c(c3)sc3ccccc34)c3ccc4oc5ccccc5c4c3)c3c2sc2ccccc23)cc1 ; c1ccc(-c2ccc(N(c3ccc4c(c3)oc3ccccc34)c3cccc4c3sc3ccccc34)c3c2sc2ccccc23)cc1]

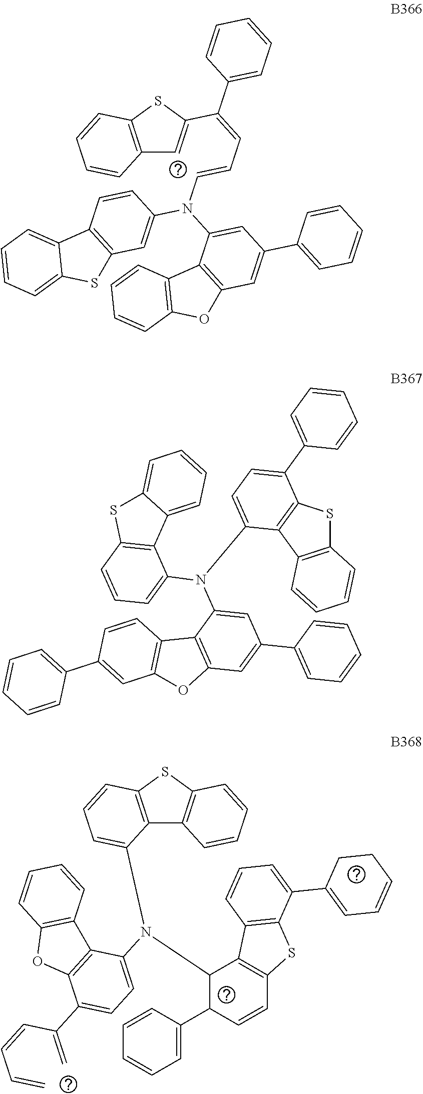 [SMILES: C(/C=C/N(c1ccc2c(c1)sc1ccccc12)c1cc(-c2ccccc2)cc2oc3ccccc3c12)=C(\c1ccccc1)c1cc2ccccc2s1 ; c1ccc(-c2ccc3c(c2)oc2cc(-c4ccccc4)cc(N(c4cccc5sc6ccccc6c45)c4ccc(-c5ccccc5)c5sc6ccccc6c45)c23)cc1 ; C=C/C=C\C(=C)c1ccc(N(c2cccc3sc4ccccc4c23)C2c3c(sc4c(C5=CC=CCC5)cccc34)C=CC2c2ccccc2)c2c1oc1ccccc12]

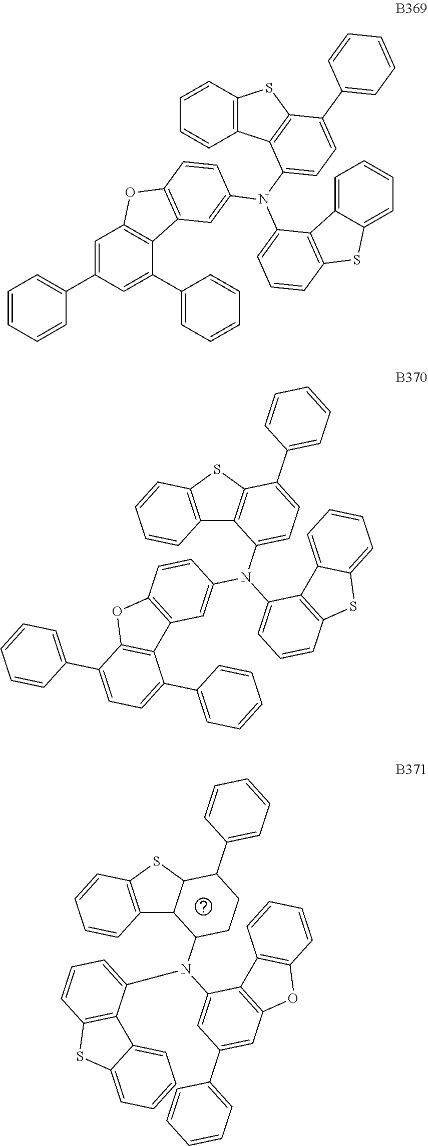 [SMILES: c1ccc(-c2cc(-c3ccccc3)c3c(c2)oc2ccc(N(c4cccc5sc6ccccc6c45)c4ccc(-c5ccccc5)c5sc6ccccc6c45)cc23)cc1 ; c1ccc(-c2ccc(-c3ccccc3)c3c2oc2ccc(N(c4cccc5sc6ccccc6c45)c4ccc(-c5ccccc5)c5sc6ccccc6c45)cc23)cc1 ; c1ccc(-c2cc(N(c3cccc4sc5ccccc5c34)C3CCC(c4ccccc4)C4Sc5ccccc5C43)c3c(c2)oc2ccccc23)cc1]

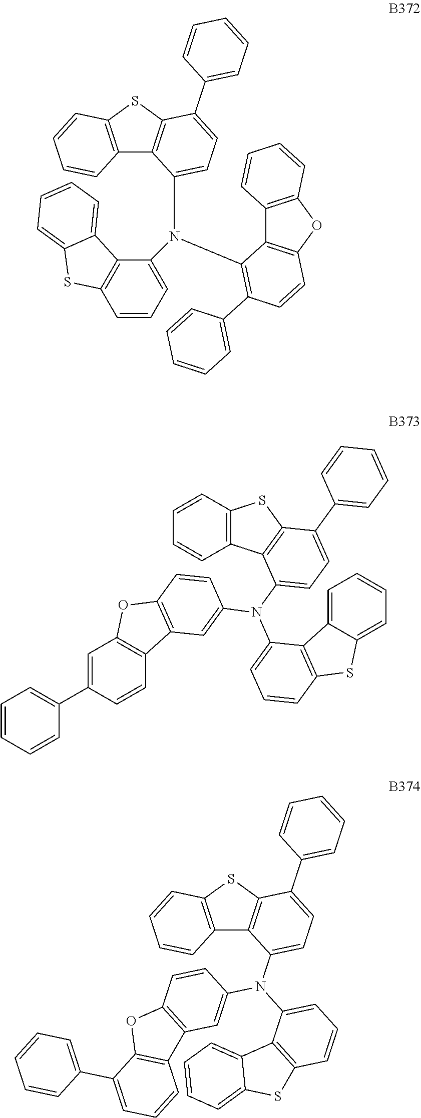 [SMILES: c1ccc(-c2ccc3oc4ccccc4c3c2N(c2cccc3sc4ccccc4c23)c2ccc(-c3ccccc3)c3sc4ccccc4c23)cc1 ; c1ccc(-c2ccc3c(c2)oc2ccc(N(c4cccc5sc6ccccc6c45)c4ccc(-c5ccccc5)c5sc6ccccc6c45)cc23)cc1 ; c1ccc(-c2cccc3c2oc2ccc(N(c4cccc5sc6ccccc6c45)c4ccc(-c5ccccc5)c5sc6ccccc6c45)cc23)cc1]

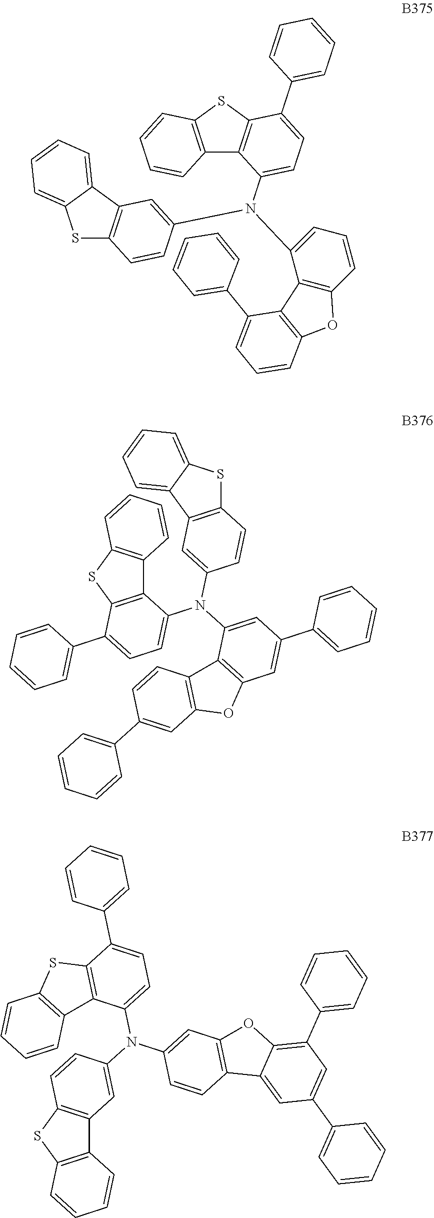 [SMILES: c1ccc(-c2ccc(N(c3ccc4sc5ccccc5c4c3)c3cccc4oc5cccc(-c6ccccc6)c5c34)c3c2sc2ccccc23)cc1 ; c1ccc(-c2ccc3c(c2)oc2cc(-c4ccccc4)cc(N(c4ccc5sc6ccccc6c5c4)c4ccc(-c5ccccc5)c5sc6ccccc6c45)c23)cc1 ; c1ccc(-c2cc(-c3ccccc3)c3oc4cc(N(c5ccc6sc7ccccc7c6c5)c5ccc(-c6ccccc6)c6sc7ccccc7c56)ccc4c3c2)cc1]

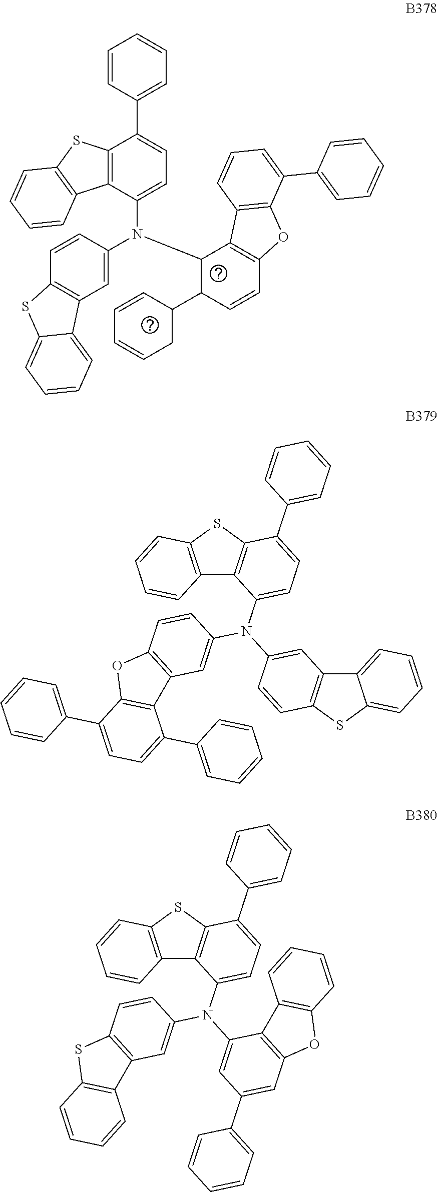 [SMILES: C1=CCC(C2C=Cc3oc4c(-c5ccccc5)cccc4c3C2N(c2ccc3sc4ccccc4c3c2)c2ccc(-c3ccccc3)c3sc4ccccc4c23)C=C1 ; c1ccc(-c2ccc(-c3ccccc3)c3c2oc2ccc(N(c4ccc5sc6ccccc6c5c4)c4ccc(-c5ccccc5)c5sc6ccccc6c45)cc23)cc1 ; c1ccc(-c2cc(N(c3ccc4sc5ccccc5c4c3)c3ccc(-c4ccccc4)c4sc5ccccc5c34)c3c(c2)oc2ccccc23)cc1]

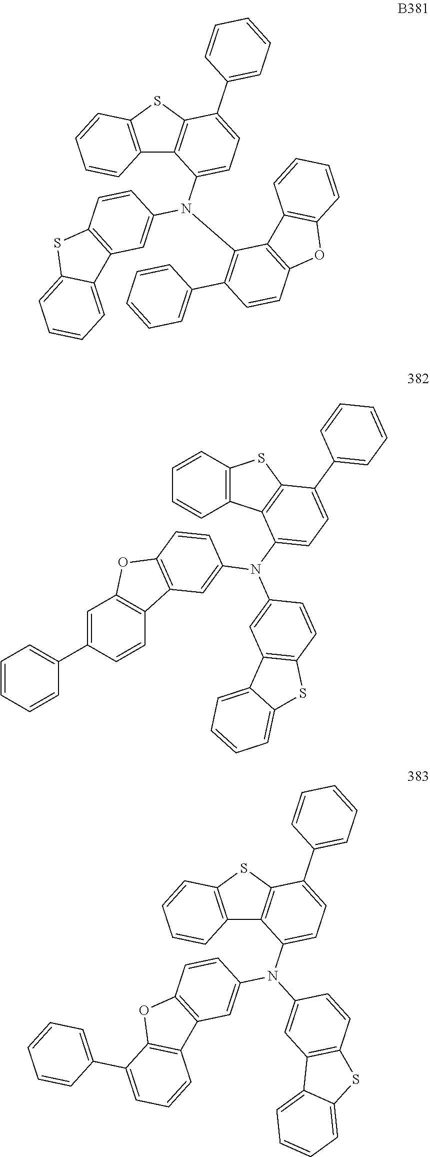 [SMILES: c1ccc(-c2ccc3oc4ccccc4c3c2N(c2ccc3sc4ccccc4c3c2)c2ccc(-c3ccccc3)c3sc4ccccc4c23)cc1 ; c1ccc(-c2ccc3c(c2)oc2ccc(N(c4ccc5sc6ccccc6c5c4)c4ccc(-c5ccccc5)c5sc6ccccc6c45)cc23)cc1 ; c1ccc(-c2cccc3c2oc2ccc(N(c4ccc5sc6ccccc6c5c4)c4ccc(-c5ccccc5)c5sc6ccccc6c45)cc23)cc1]

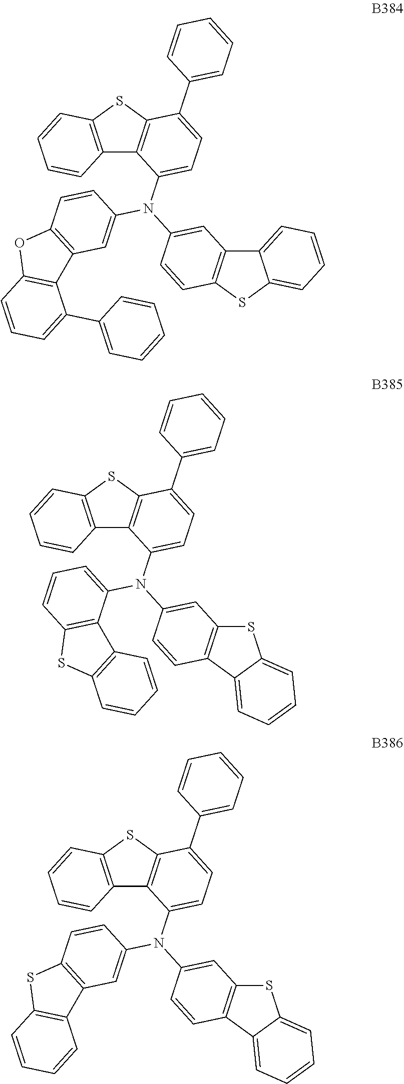

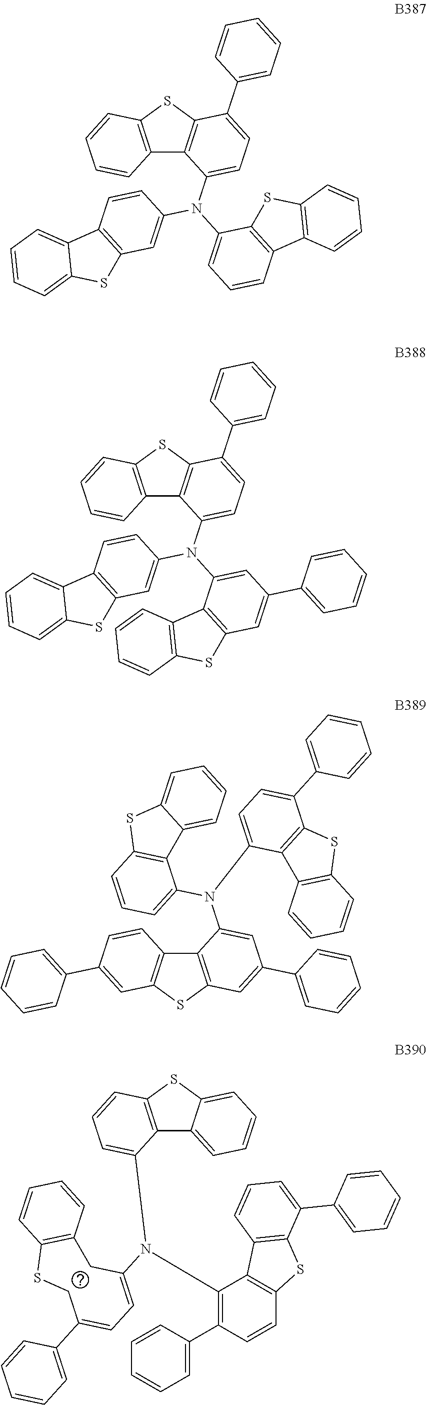 [SMILES: c1ccc(-c2ccc(N(c3ccc4c(c3)sc3ccccc34)c3cccc4c3sc3ccccc34)c3c2sc2ccccc23)cc1 ; c1ccc(-c2cc(N(c3ccc4c(c3)sc3ccccc34)c3ccc(-c4ccccc4)c4sc5ccccc5c34)c3c(c2)sc2ccccc23)cc1 ; c1ccc(-c2ccc3c(c2)sc2cc(-c4ccccc4)cc(N(c4cccc5sc6ccccc6c45)c4ccc(-c5ccccc5)c5sc6ccccc6c45)c23)cc1 ; C1=C(/c2ccccc2)CSc2ccccc2C\C(N(c2cccc3sc4ccccc4c23)c2c(-c3ccccc3)ccc3sc4c(-c5ccccc5)cccc4c23)=C/1]

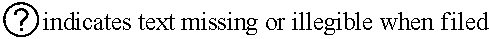

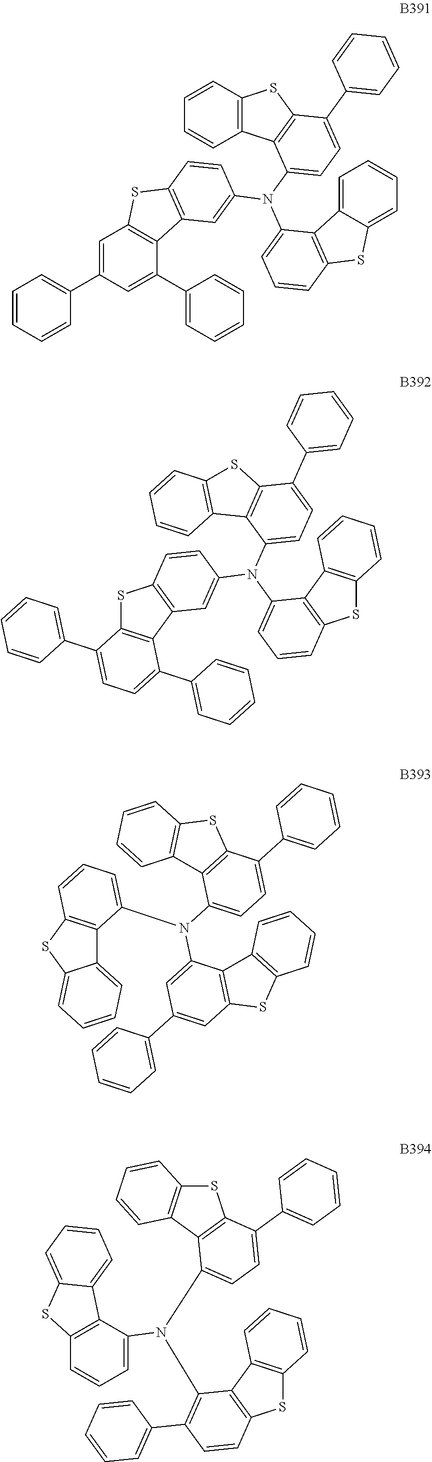 [SMILES: c1ccc(-c2cc(-c3ccccc3)c3c(c2)sc2ccc(N(c4cccc5sc6ccccc6c45)c4ccc(-c5ccccc5)c5sc6ccccc6c45)cc23)cc1 ; c1ccc(-c2ccc(-c3ccccc3)c3c2sc2ccc(N(c4cccc5sc6ccccc6c45)c4ccc(-c5ccccc5)c5sc6ccccc6c45)cc23)cc1 ; c1ccc(-c2cc(N(c3cccc4sc5ccccc5c34)c3ccc(-c4ccccc4)c4sc5ccccc5c34)c3c(c2)sc2ccccc23)cc1 ; c1ccc(-c2ccc3sc4ccccc4c3c2N(c2cccc3sc4ccccc4c23)c2ccc(-c3ccccc3)c3sc4ccccc4c23)cc1]

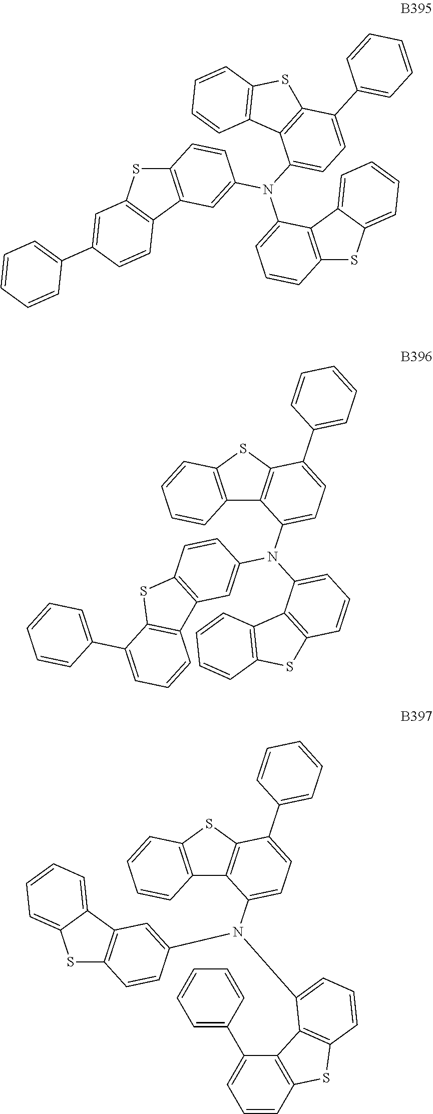 [SMILES: c1ccc(-c2ccc3c(c2)sc2ccc(N(c4cccc5sc6ccccc6c45)c4ccc(-c5ccccc5)c5sc6ccccc6c45)cc23)cc1 ; c1ccc(-c2cccc3c2sc2ccc(N(c4cccc5sc6ccccc6c45)c4ccc(-c5ccccc5)c5sc6ccccc6c45)cc23)cc1 ; c1ccc(-c2ccc(N(c3ccc4sc5ccccc5c4c3)c3cccc4sc5cccc(-c6ccccc6)c5c34)c3c2sc2ccccc23)cc1]

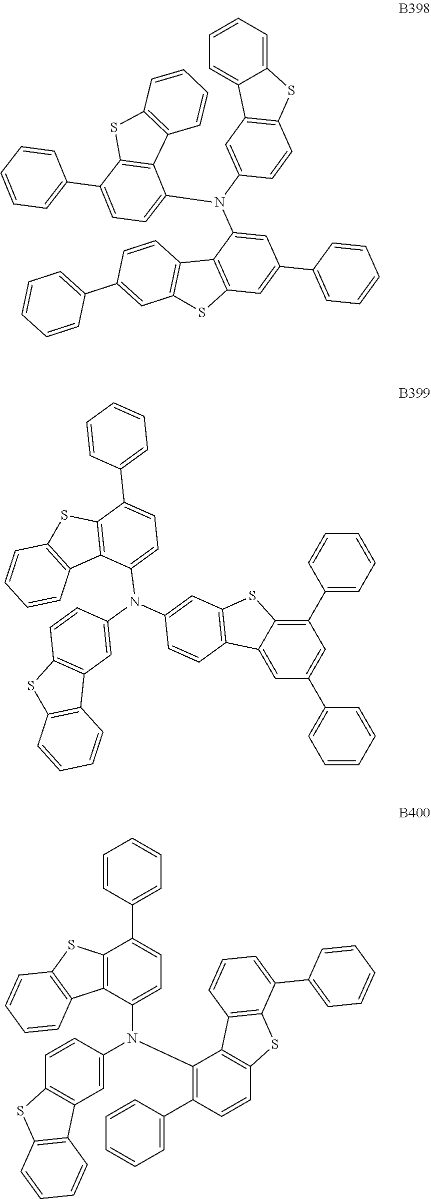 [SMILES: c1ccc(-c2ccc3c(c2)sc2cc(-c4ccccc4)cc(N(c4ccc5sc6ccccc6c5c4)c4ccc(-c5ccccc5)c5sc6ccccc6c45)c23)cc1 ; c1ccc(-c2cc(-c3ccccc3)c3sc4cc(N(c5ccc6sc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3sc4ccccc4c3c2)c2ccc(-c3ccccc3)c3sc4ccccc4c23)cc1]

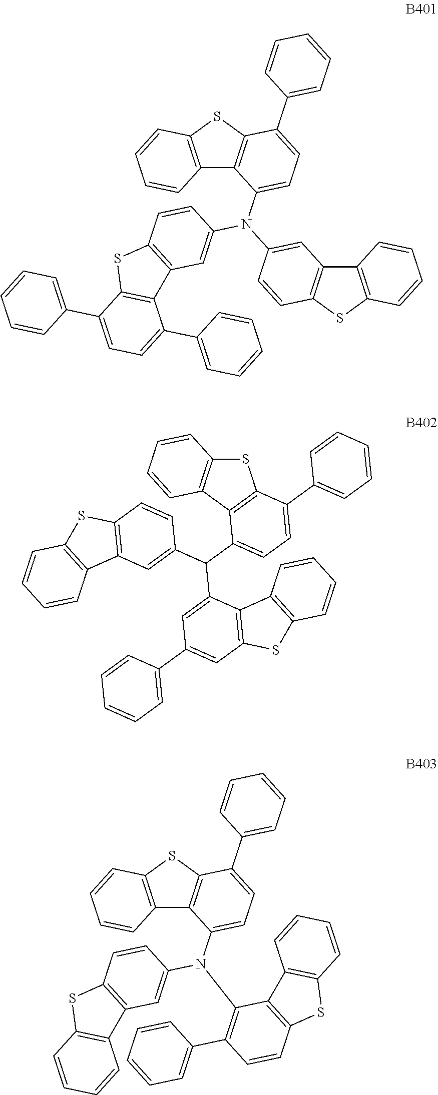 [SMILES: c1ccc(-c2ccc(-c3ccccc3)c3c2sc2ccc(N(c4ccc5sc6ccccc6c5c4)c4ccc(-c5ccccc5)c5sc6ccccc6c45)cc23)cc1 ; c1ccc(-c2cc(C(c3ccc4sc5ccccc5c4c3)c3ccc(-c4ccccc4)c4sc5ccccc5c34)c3c(c2)sc2ccccc23)cc1 ; c1ccc(-c2ccc3sc4ccccc4c3c2N(c2ccc3sc4ccccc4c3c2)c2ccc(-c3ccccc3)c3sc4ccccc4c23)cc1]

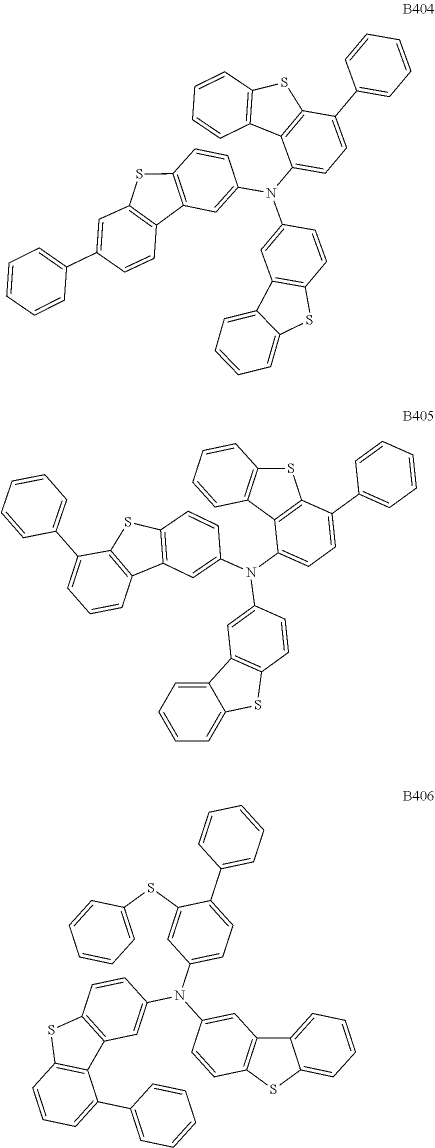 [SMILES: c1ccc(-c2ccc3c(c2)sc2ccc(N(c4ccc5sc6ccccc6c5c4)c4ccc(-c5ccccc5)c5sc6ccccc6c45)cc23)cc1 ; c1ccc(-c2cccc3c2sc2ccc(N(c4ccc5sc6ccccc6c5c4)c4ccc(-c5ccccc5)c5sc6ccccc6c45)cc23)cc1 ; c1ccc(Sc2cc(N(c3ccc4sc5ccccc5c4c3)c3ccc4sc5cccc(-c6ccccc6)c5c4c3)ccc2-c2ccccc2)cc1]

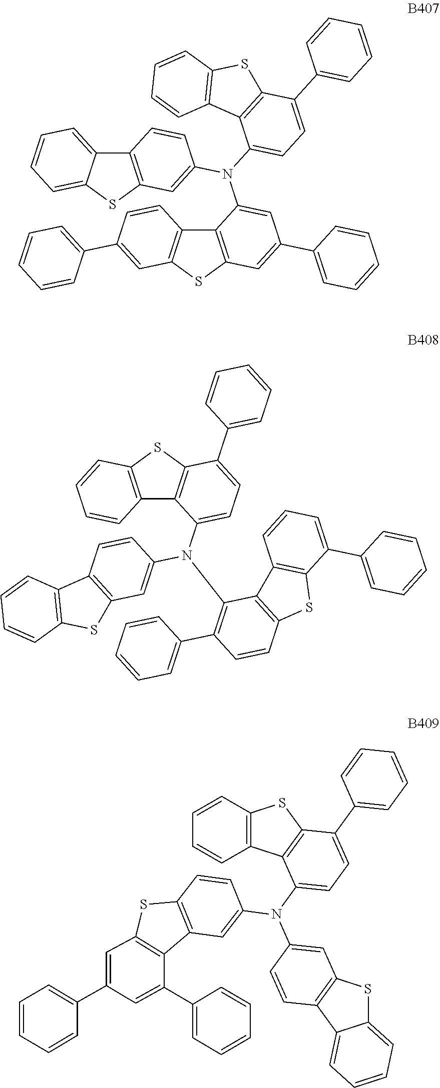 [SMILES: c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1 ; c1ccc(-c2ccc3sc4c(-c5ccccc5)cccc4c3c2N(c2ccc3c(c2)sc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1]

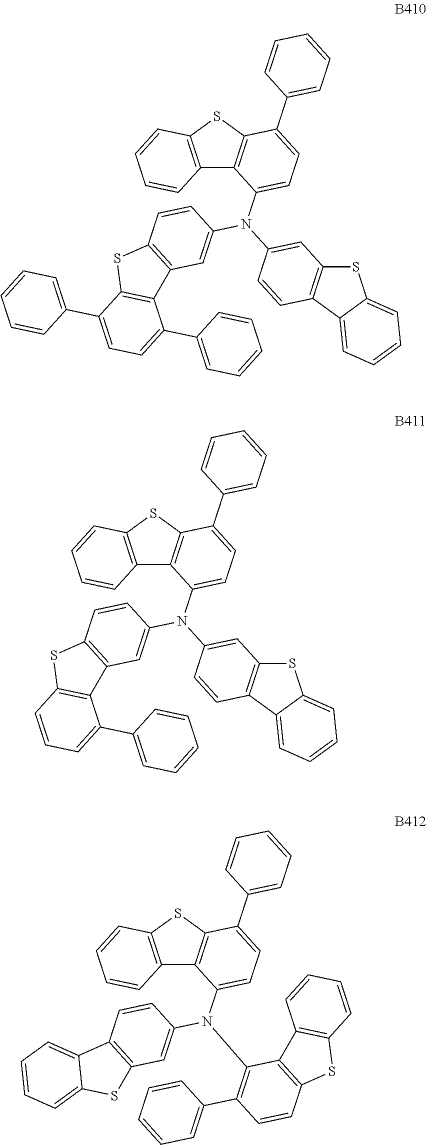 [SMILES: c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1 ; c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1]

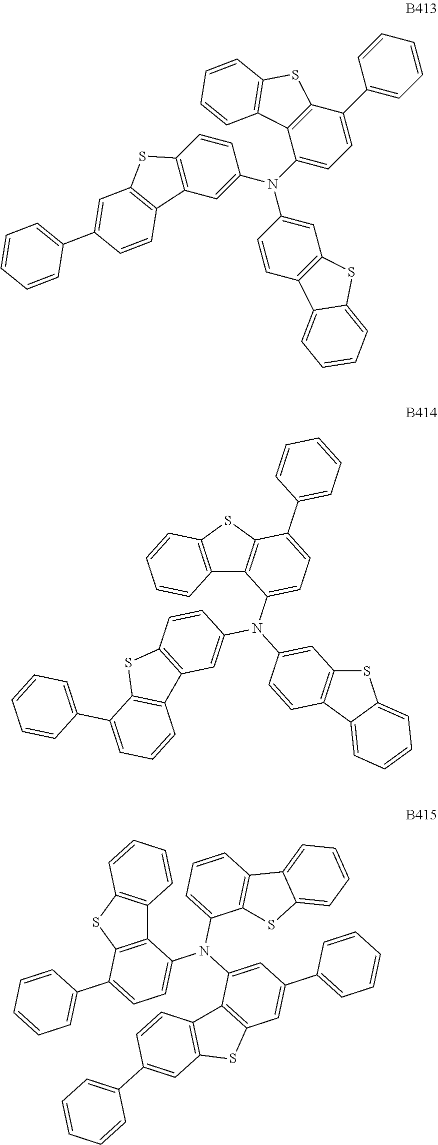 [SMILES: c1ccc(-c2ccc3c(c2)sc2ccc(N(c4ccc5c(c4)sc4ccccc45)c4ccc(-c5ccccc5)c5sc6ccccc6c45)cc23)cc1 ; c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4ccc(-c5ccccc5)c5sc6ccccc6c45)c23)cc1]

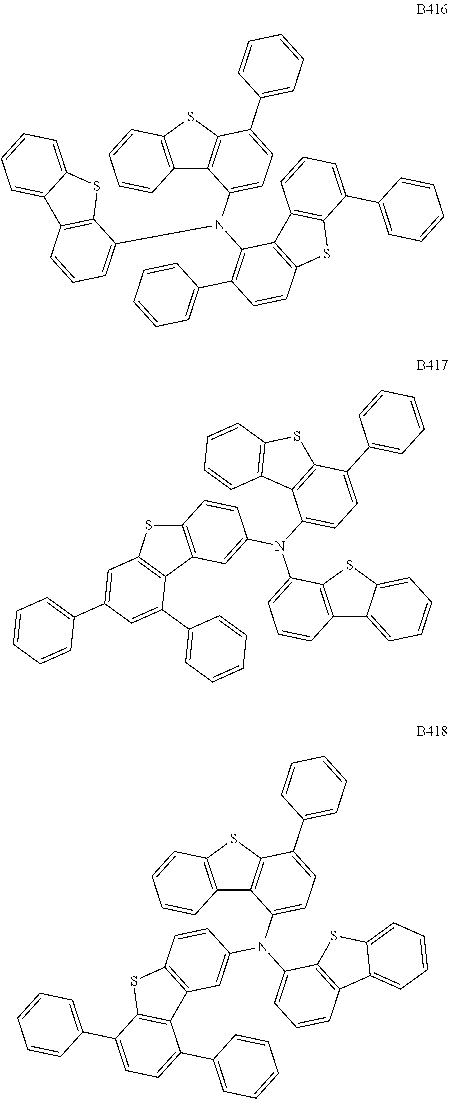 [SMILES: c1ccc(-c2ccc3sc4c(-c5ccccc5)cccc4c3c2N(c2cccc3c2sc2ccccc23)c2ccc(-c3ccccc3)c3sc4ccccc4c23)cc1 ; c1ccc(-c2cc(-c3ccccc3)c3c(c2)sc2ccc(N(c4cccc5c4sc4ccccc45)c4ccc(-c5ccccc5)c5sc6ccccc6c45)cc23)cc1 ; c1ccc(-c2ccc(-c3ccccc3)c3c2sc2ccc(N(c4cccc5c4sc4ccccc45)c4ccc(-c5ccccc5)c5sc6ccccc6c45)cc23)cc1]

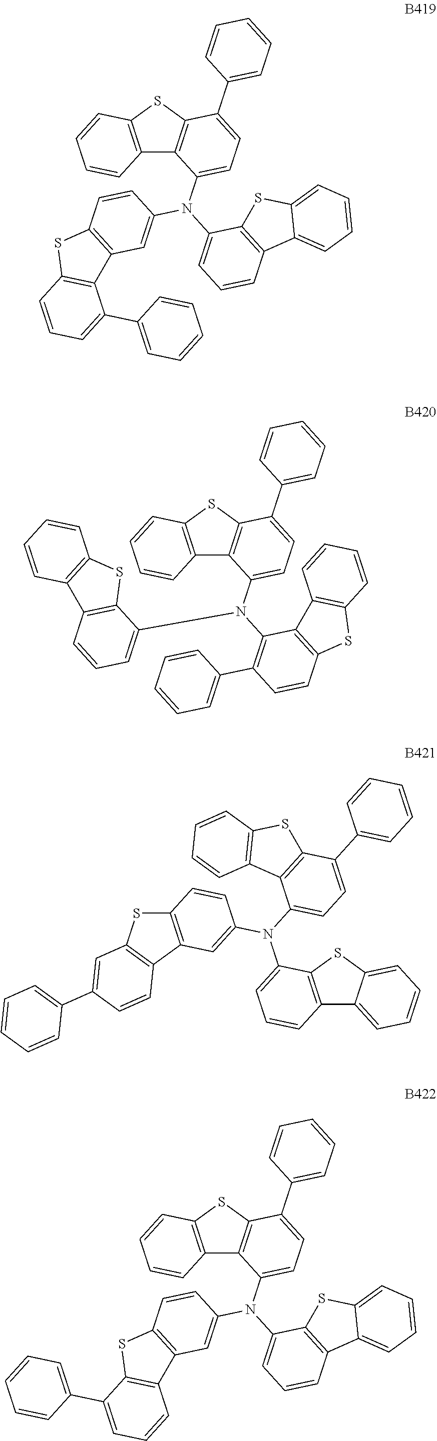 [SMILES: c1ccc(-c2ccc(N(c3ccc4sc5cccc(-c6ccccc6)c5c4c3)c3cccc4c3sc3ccccc34)c3c2sc2ccccc23)cc1 ; c1ccc(-c2ccc3sc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2cccc3c2sc2ccc(N(c4cccc5c4sc4ccccc45)c4ccc(-c5ccccc5)c5sc6ccccc6c45)cc23)cc1]

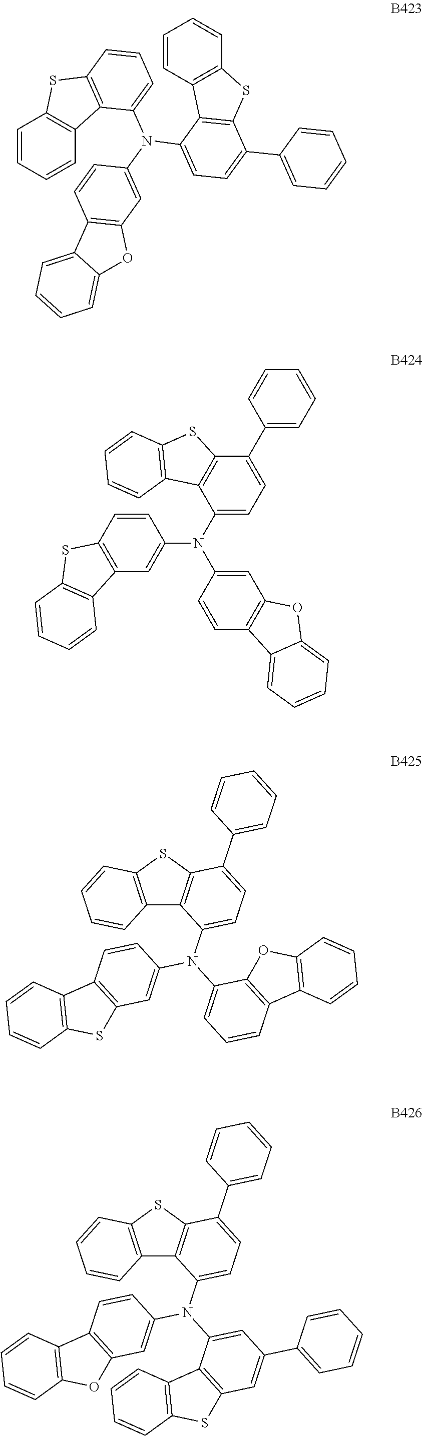 [SMILES: c1ccc(-c2ccc(N(c3ccc4c(c3)oc3ccccc34)c3cccc4sc5ccccc5c34)c3c2sc2ccccc23)cc1 ; c1ccc(-c2ccc(N(c3ccc4c(c3)oc3ccccc34)c3ccc4sc5ccccc5c4c3)c3c2sc2ccccc23)cc1 ; c1ccc(-c2ccc(N(c3ccc4c(c3)sc3ccccc34)c3cccc4c3oc3ccccc34)c3c2sc2ccccc23)cc1 ; c1ccc(-c2cc(N(c3ccc4c(c3)oc3ccccc34)c3ccc(-c4ccccc4)c4sc5ccccc5c34)c3c(c2)sc2ccccc23)cc1]

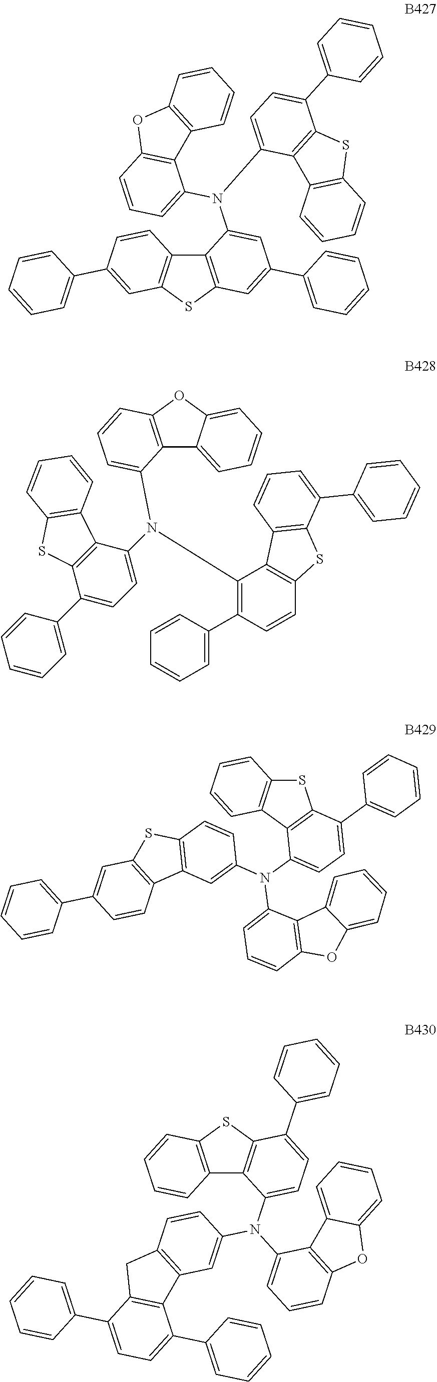 [SMILES: c1ccc(-c2ccc3c(c2)sc2cc(-c4ccccc4)cc(N(c4cccc5oc6ccccc6c45)c4ccc(-c5ccccc5)c5sc6ccccc6c45)c23)cc1 ; c1ccc(-c2ccc3sc4c(-c5ccccc5)cccc4c3c2N(c2cccc3oc4ccccc4c23)c2ccc(-c3ccccc3)c3sc4ccccc4c23)cc1 ; c1ccc(-c2ccc3c(c2)sc2ccc(N(c4cccc5oc6ccccc6c45)c4ccc(-c5ccccc5)c5sc6ccccc6c45)cc23)cc1 ; c1ccc(-c2ccc(-c3ccccc3)c3c2Cc2ccc(N(c4cccc5oc6ccccc6c45)c4ccc(-c5ccccc5)c5sc6ccccc6c45)cc2-3)cc1]

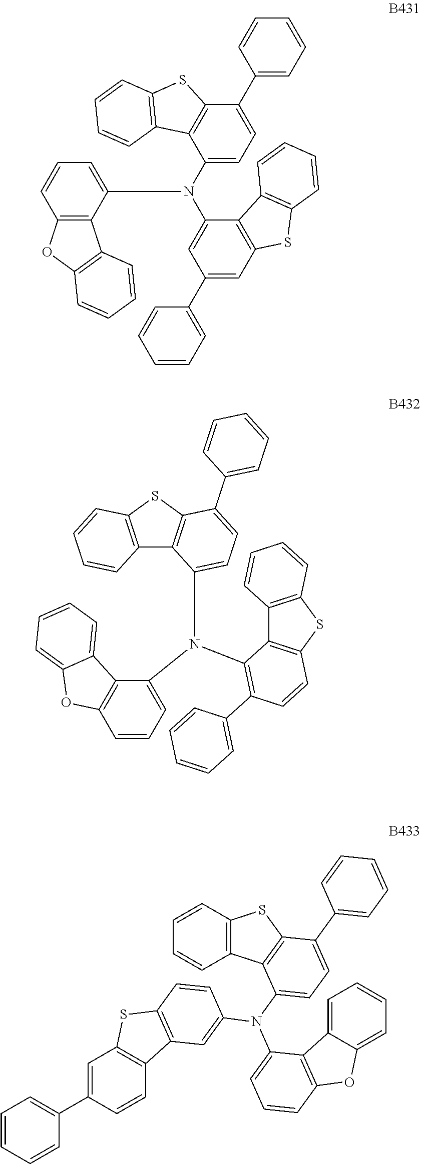 [SMILES: c1ccc(-c2cc(N(c3cccc4oc5ccccc5c34)c3ccc(-c4ccccc4)c4sc5ccccc5c34)c3c(c2)sc2ccccc23)cc1 ; c1ccc(-c2ccc3sc4ccccc4c3c2N(c2cccc3oc4ccccc4c23)c2ccc(-c3ccccc3)c3sc4ccccc4c23)cc1 ; c1ccc(-c2ccc3c(c2)sc2ccc(N(c4cccc5oc6ccccc6c45)c4ccc(-c5ccccc5)c5sc6ccccc6c45)cc23)cc1]

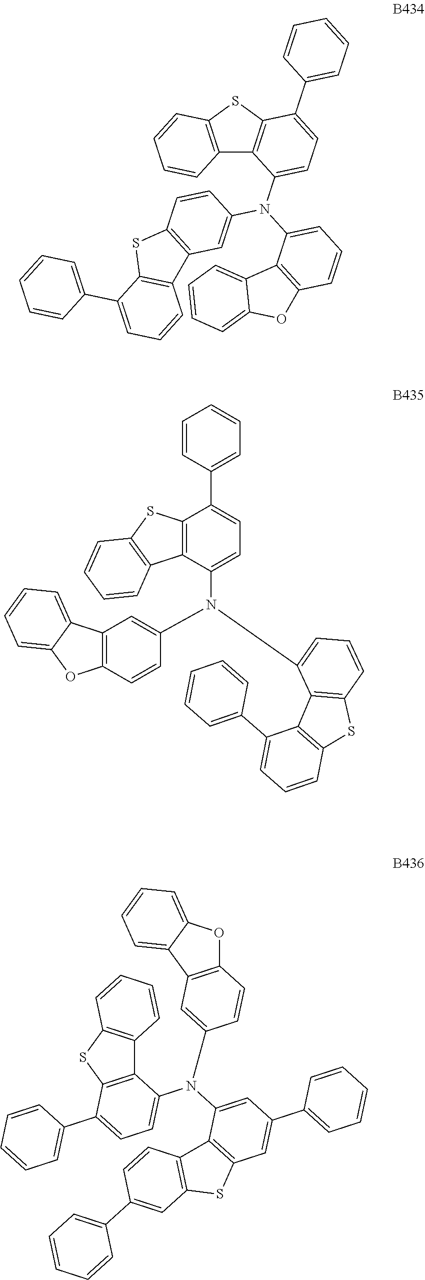 [SMILES: c1ccc(-c2cccc3c2sc2ccc(N(c4cccc5oc6ccccc6c45)c4ccc(-c5ccccc5)c5sc6ccccc6c45)cc23)cc1 ; c1ccc(-c2ccc(N(c3ccc4oc5ccccc5c4c3)c3cccc4sc5cccc(-c6ccccc6)c5c34)c3c2sc2ccccc23)cc1 ; c1ccc(-c2ccc3c(c2)sc2cc(-c4ccccc4)cc(N(c4ccc5oc6ccccc6c5c4)c4ccc(-c5ccccc5)c5sc6ccccc6c45)c23)cc1]

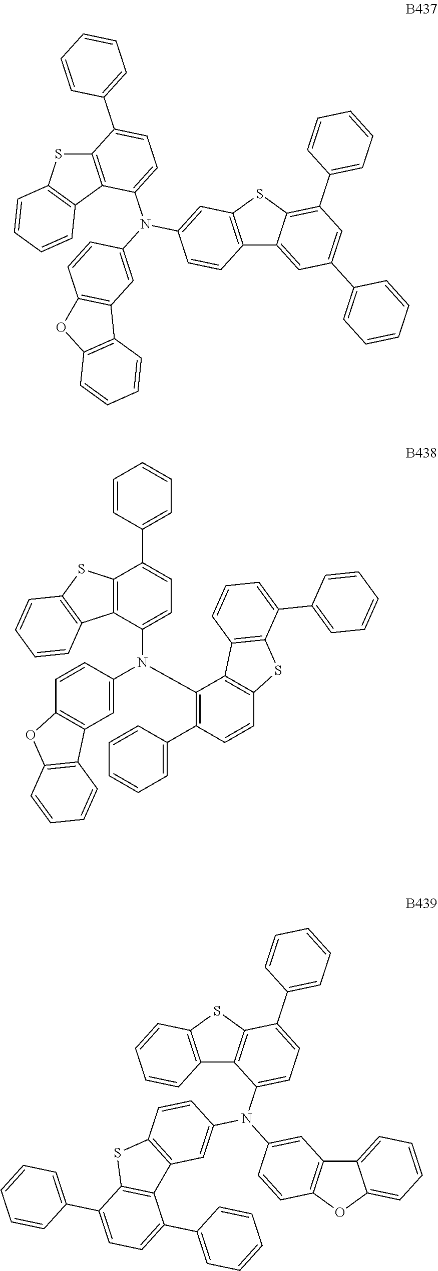 [SMILES: c1ccc(-c2cc(-c3ccccc3)c3sc4cc(N(c5ccc6oc7ccccc7c6c5)c5ccc(-c6ccccc6)c6sc7ccccc7c56)ccc4c3c2)cc1 ; c1ccc(-c2ccc3sc4c(-c5ccccc5)cccc4c3c2N(c2ccc3oc4ccccc4c3c2)c2ccc(-c3ccccc3)c3sc4ccccc4c23)cc1 ; c1ccc(-c2ccc(-c3ccccc3)c3c2sc2ccc(N(c4ccc5oc6ccccc6c5c4)c4ccc(-c5ccccc5)c5sc6ccccc6c45)cc23)cc1]

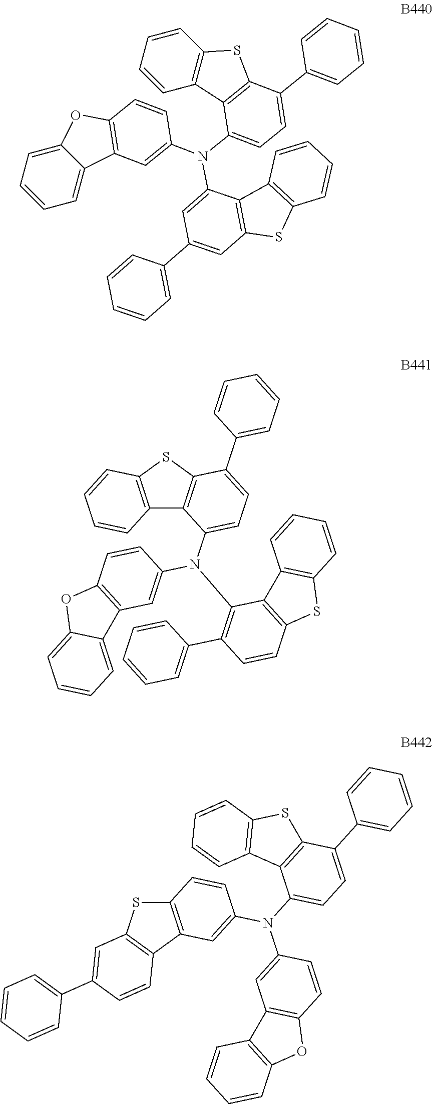 [SMILES: c1ccc(-c2cc(N(c3ccc4oc5ccccc5c4c3)c3ccc(-c4ccccc4)c4sc5ccccc5c34)c3c(c2)sc2ccccc23)cc1 ; c1ccc(-c2ccc3sc4ccccc4c3c2N(c2ccc3oc4ccccc4c3c2)c2ccc(-c3ccccc3)c3sc4ccccc4c23)cc1 ; c1ccc(-c2ccc3c(c2)sc2ccc(N(c4ccc5oc6ccccc6c5c4)c4ccc(-c5ccccc5)c5sc6ccccc6c45)cc23)cc1]

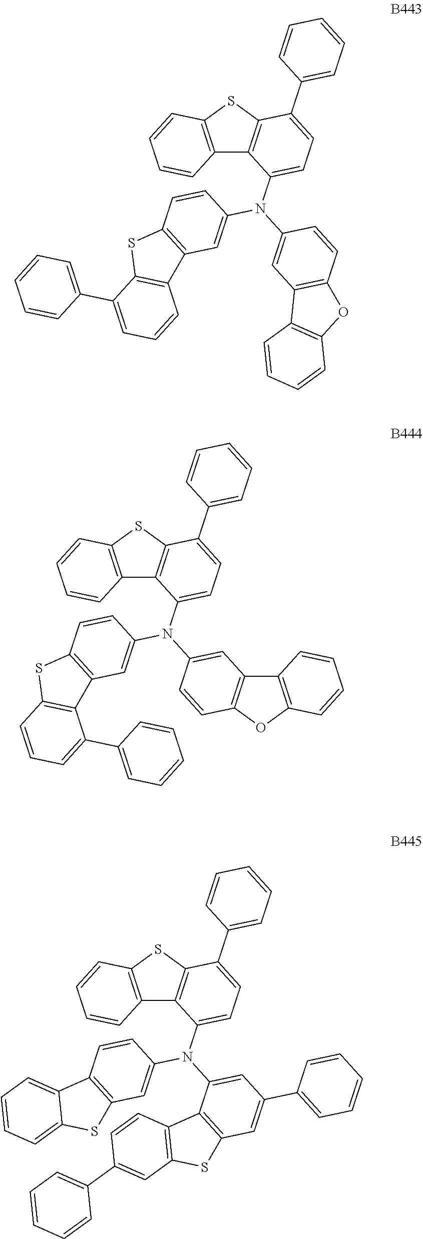 [SMILES: c1ccc(-c2cccc3c2sc2ccc(N(c4ccc5oc6ccccc6c5c4)c4ccc(-c5ccccc5)c5sc6ccccc6c45)cc23)cc1 ; c1ccc(-c2ccc(N(c3ccc4oc5ccccc5c4c3)c3ccc4sc5cccc(-c6ccccc6)c5c4c3)c3c2sc2ccccc23)cc1 ; c1ccc(-c2ccc3c(c2)sc2cc(-c4ccccc4)cc(N(c4ccc5c(c4)sc4ccccc45)c4ccc(-c5ccccc5)c5sc6ccccc6c45)c23)cc1]

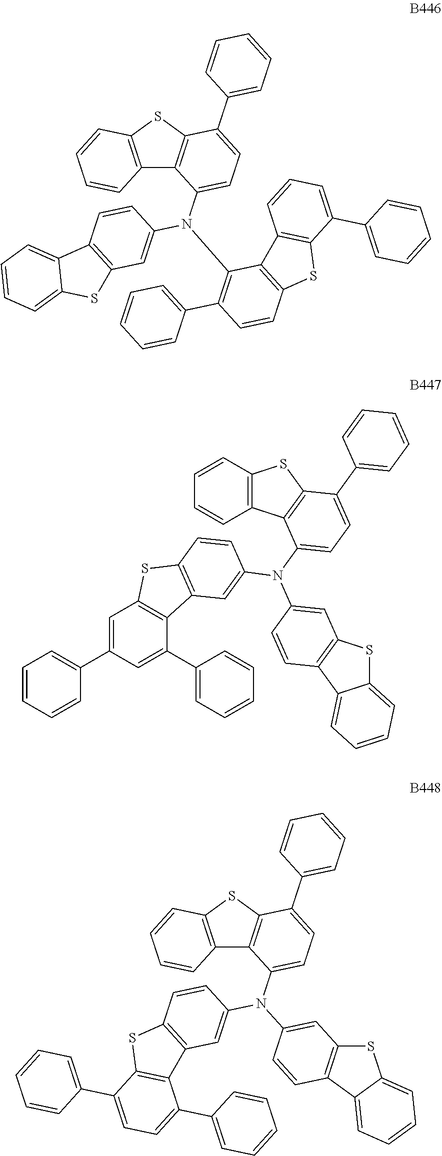 [SMILES: c1ccc(-c2ccc3sc4c(-c5ccccc5)cccc4c3c2N(c2ccc3c(c2)sc2ccccc23)c2ccc(-c3ccccc3)c3sc4ccccc4c23)cc1 ; c1ccc(-c2cc(-c3ccccc3)c3c(c2)sc2ccc(N(c4ccc5c(c4)sc4ccccc45)c4ccc(-c5ccccc5)c5sc6ccccc6c45)cc23)cc1 ; c1ccc(-c2ccc(-c3ccccc3)c3c2sc2ccc(N(c4ccc5c(c4)sc4ccccc45)c4ccc(-c5ccccc5)c5sc6ccccc6c45)cc23)cc1]

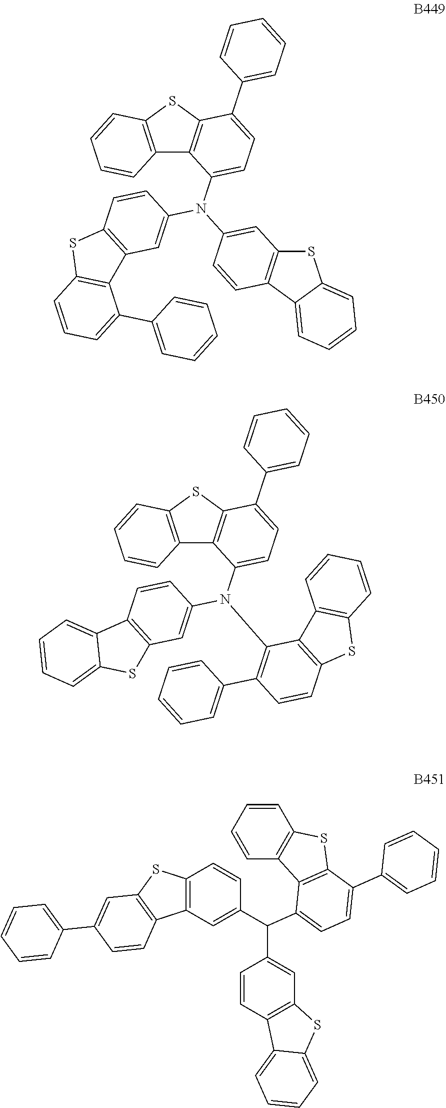 [SMILES: c1ccc(-c2ccc(N(c3ccc4c(c3)sc3ccccc34)c3ccc4sc5cccc(-c6ccccc6)c5c4c3)c3c2sc2ccccc23)cc1 ; c1ccc(-c2ccc3sc4ccccc4c3c2N(c2ccc3c(c2)sc2ccccc23)c2ccc(-c3ccccc3)c3sc4ccccc4c23)cc1 ; c1ccc(-c2ccc3c(c2)sc2ccc(C(c4ccc5c(c4)sc4ccccc45)c4ccc(-c5ccccc5)c5sc6ccccc6c45)cc23)cc1]

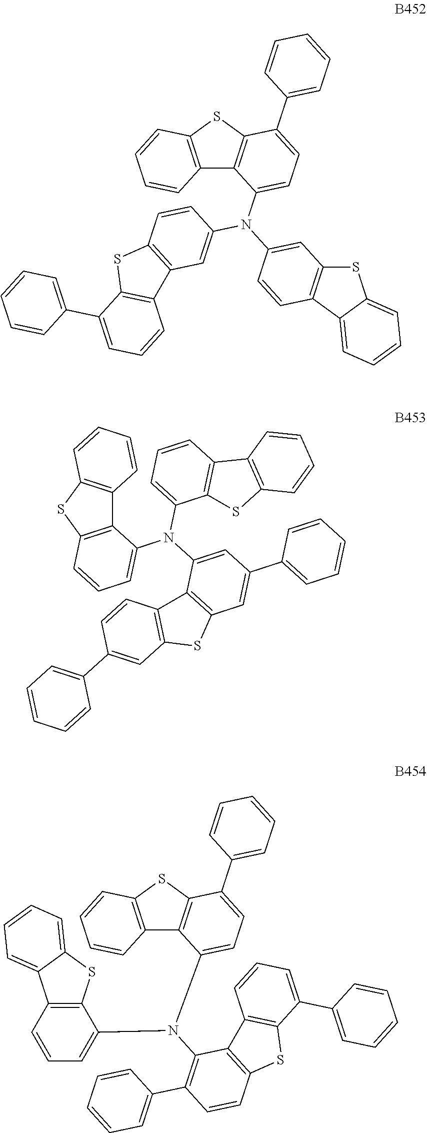 [SMILES: c1ccc(-c2cccc3c2sc2ccc(N(c4ccc5c(c4)sc4ccccc45)c4ccc(-c5ccccc5)c5sc6ccccc6c45)cc23)cc1 ; c1ccc(-c2ccc3c(c2)sc2cc(-c4ccccc4)cc(N(c4cccc5c4sc4ccccc45)c4cccc5sc6ccccc6c45)c23)cc1 ; c1ccc(-c2ccc3sc4c(-c5ccccc5)cccc4c3c2N(c2cccc3c2sc2ccccc23)c2ccc(-c3ccccc3)c3sc4ccccc4c23)cc1]

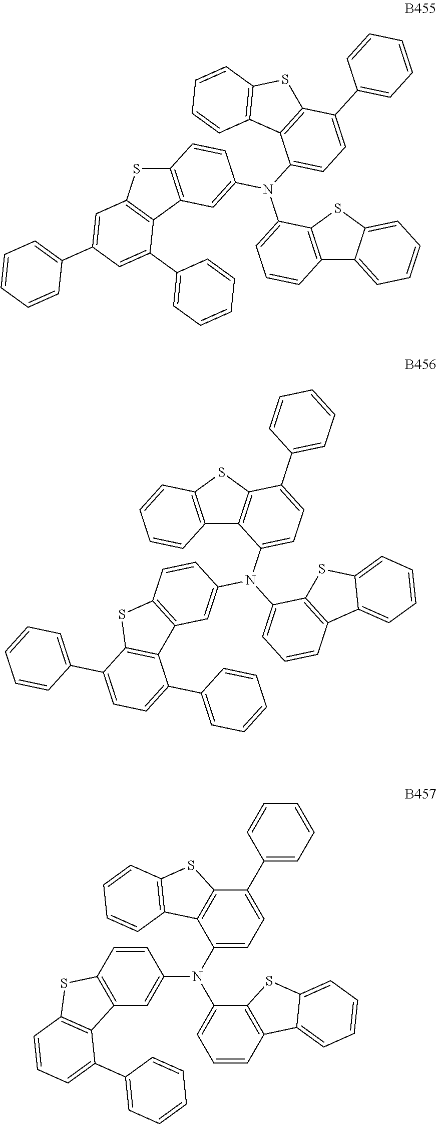 [SMILES: c1ccc(-c2cc(-c3ccccc3)c3c(c2)sc2ccc(N(c4cccc5c4sc4ccccc45)c4ccc(-c5ccccc5)c5sc6ccccc6c45)cc23)cc1 ; c1ccc(-c2ccc(-c3ccccc3)c3c2sc2ccc(N(c4cccc5c4sc4ccccc45)c4ccc(-c5ccccc5)c5sc6ccccc6c45)cc23)cc1 ; c1ccc(-c2ccc(N(c3ccc4sc5cccc(-c6ccccc6)c5c4c3)c3cccc4c3sc3ccccc34)c3c2sc2ccccc23)cc1]

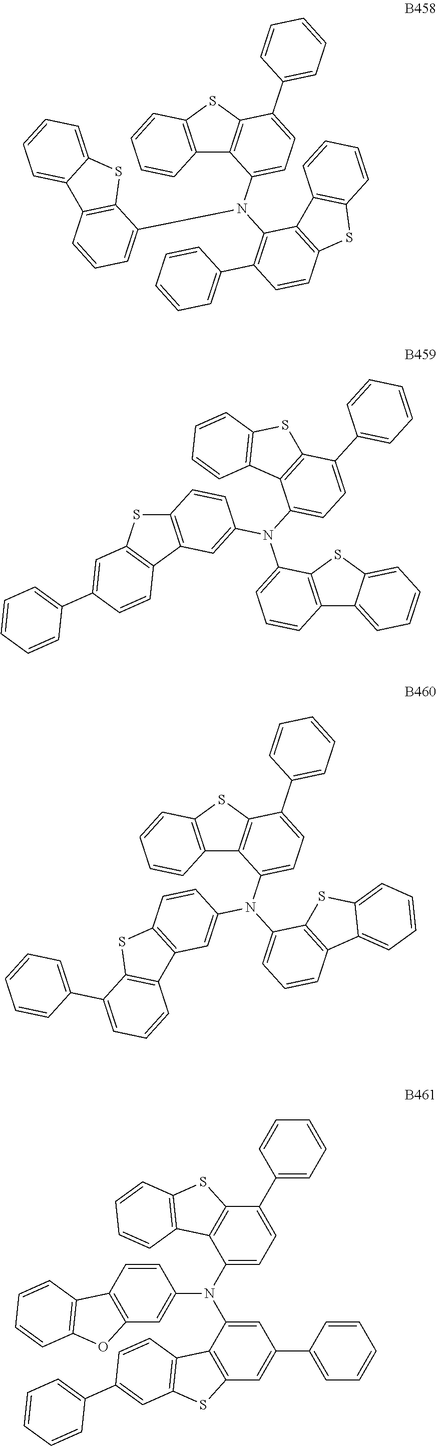 [SMILES: c1ccc(-c2ccc3sc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4sc4ccccc45)c4ccc(-c5ccccc5)c5sc6ccccc6c45)cc23)cc1 ; c1ccc(-c2cccc3c2sc2ccc(N(c4cccc5c4sc4ccccc45)c4ccc(-c5ccccc5)c5sc6ccccc6c45)cc23)cc1 ; c1ccc(-c2ccc3c(c2)sc2cc(-c4ccccc4)cc(N(c4ccc5c(c4)oc4ccccc45)c4ccc(-c5ccccc5)c5sc6ccccc6c45)c23)cc1]

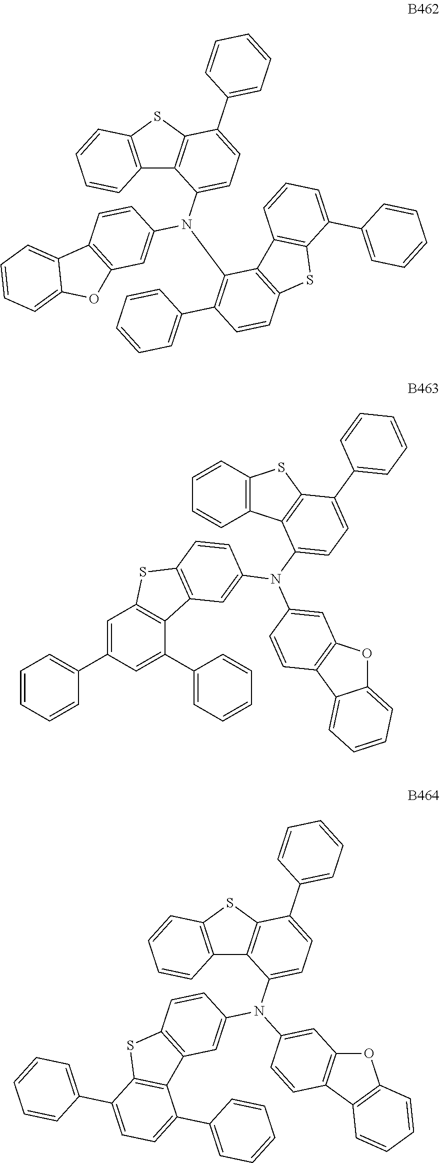

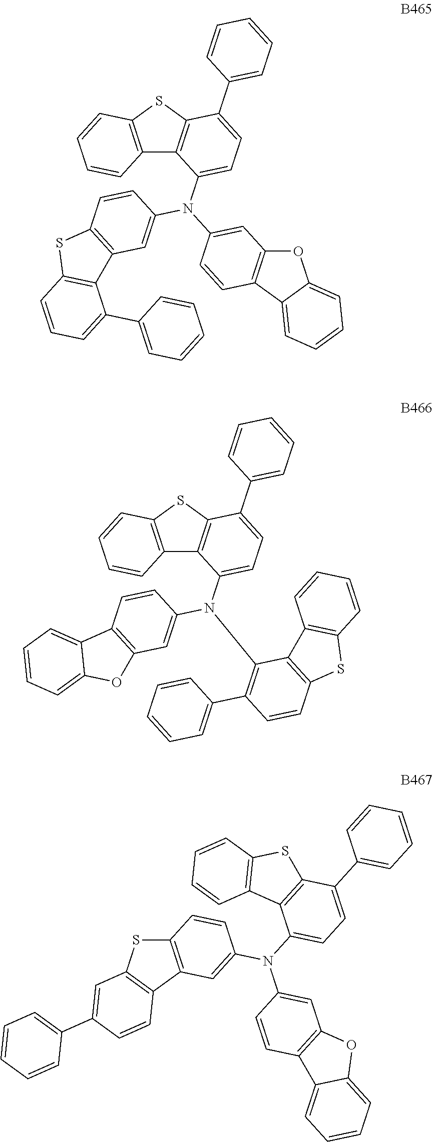

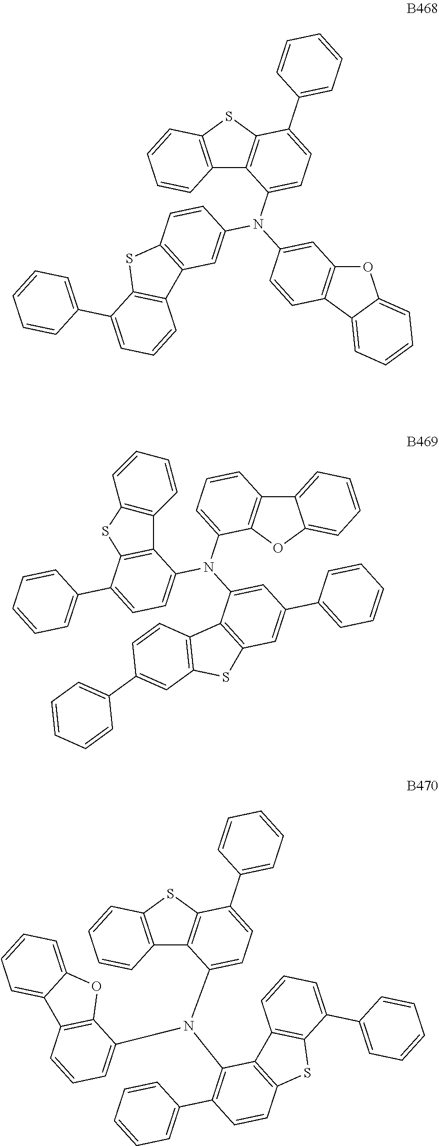

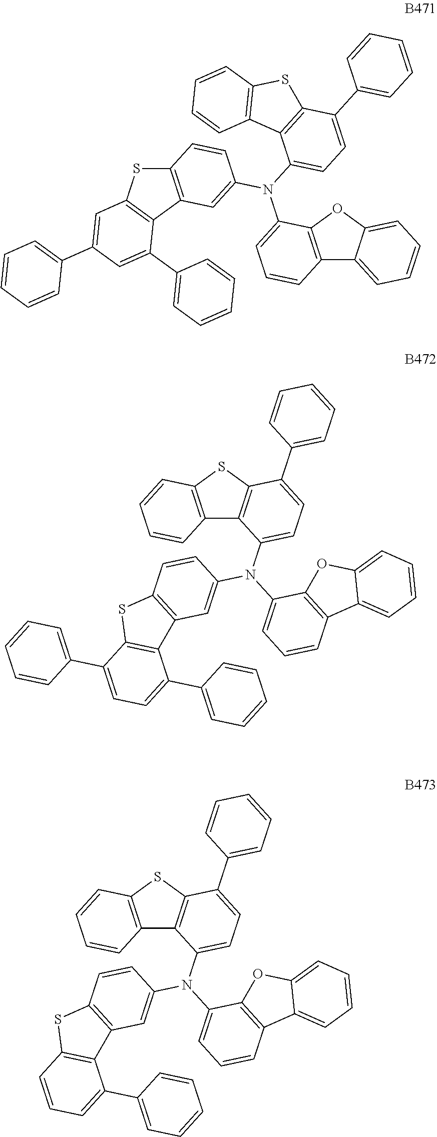 [SMILES: c1ccc(-c2cc(-c3ccccc3)c3c(c2)sc2ccc(N(c4cccc5c4oc4ccccc45)c4ccc(-c5ccccc5)c5sc6ccccc6c45)cc23)cc1 ; c1ccc(-c2ccc(-c3ccccc3)c3c2sc2ccc(N(c4cccc5c4oc4ccccc45)c4ccc(-c5ccccc5)c5sc6ccccc6c45)cc23)cc1 ; c1ccc(-c2ccc(N(c3ccc4sc5cccc(-c6ccccc6)c5c4c3)c3cccc4c3oc3ccccc34)c3c2sc2ccccc23)cc1]

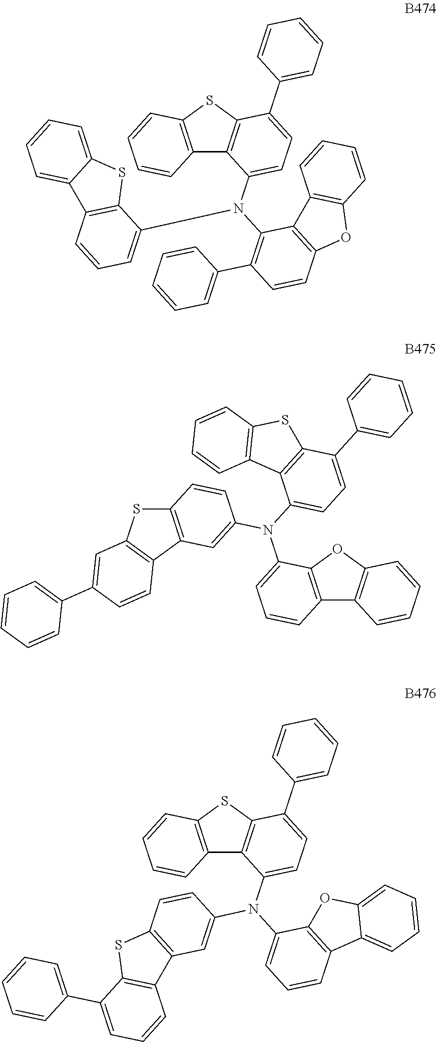 [SMILES: c1ccc(-c2ccc3oc4ccccc4c3c2N(c2cccc3c2sc2ccccc23)c2ccc(-c3ccccc3)c3sc4ccccc4c23)cc1 ; c1ccc(-c2ccc3c(c2)sc2ccc(N(c4cccc5c4oc4ccccc45)c4ccc(-c5ccccc5)c5sc6ccccc6c45)cc23)cc1 ; c1ccc(-c2cccc3c2sc2ccc(N(c4cccc5c4oc4ccccc45)c4ccc(-c5ccccc5)c5sc6ccccc6c45)cc23)cc1]

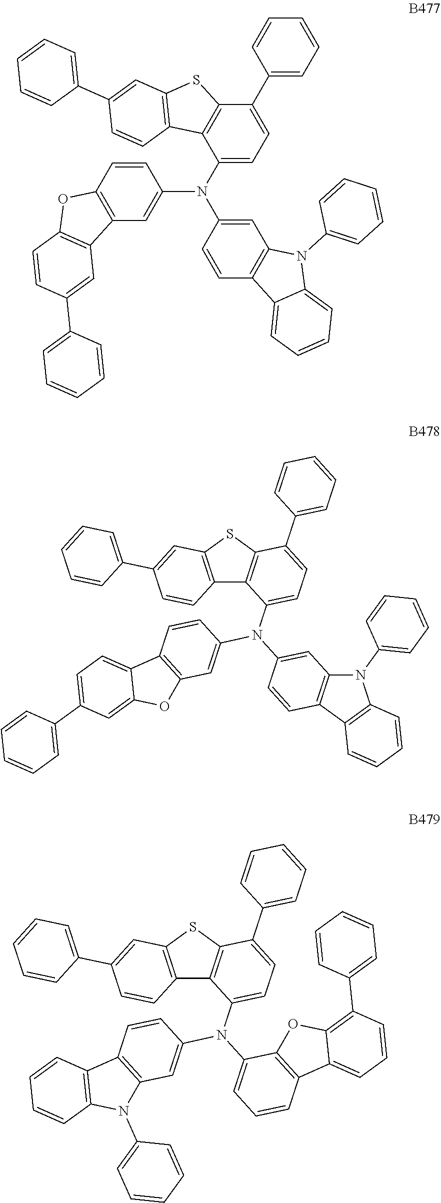 [SMILES: c1ccc(-c2ccc3c(c2)sc2c(-c4ccccc4)ccc(N(c4ccc5oc6ccc(-c7ccccc7)cc6c5c4)c4ccc5c6ccccc6n(-c6ccccc6)c5c4)c23)cc1 ; c1ccc(-c2ccc3c(c2)oc2cc(N(c4ccc5c6ccccc6n(-c6ccccc6)c5c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c6ccccc6n(-c6ccccc6)c5c4)c4cccc5c4oc4c(-c6ccccc6)cccc45)c23)cc1]

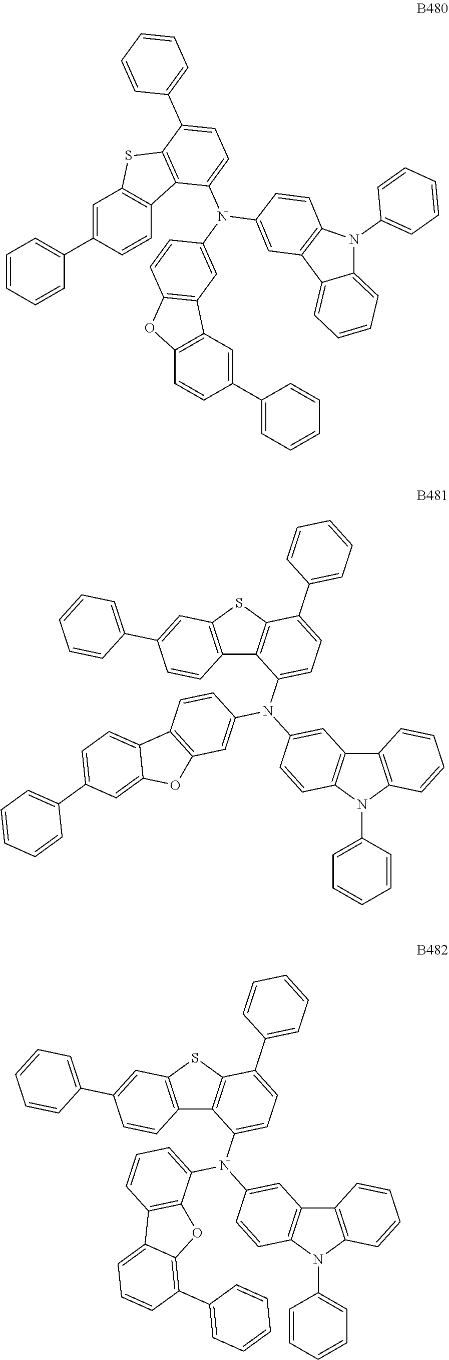 [SMILES: c1ccc(-c2ccc3c(c2)sc2c(-c4ccccc4)ccc(N(c4ccc5oc6ccc(-c7ccccc7)cc6c5c4)c4ccc5c(c4)c4ccccc4n5-c4ccccc4)c23)cc1 ; c1ccc(-c2ccc3c(c2)oc2cc(N(c4ccc5c(c4)c4ccccc4n5-c4ccccc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c(c4)c4ccccc4n5-c4ccccc4)c4cccc5c4oc4c(-c6ccccc6)cccc45)c23)cc1]

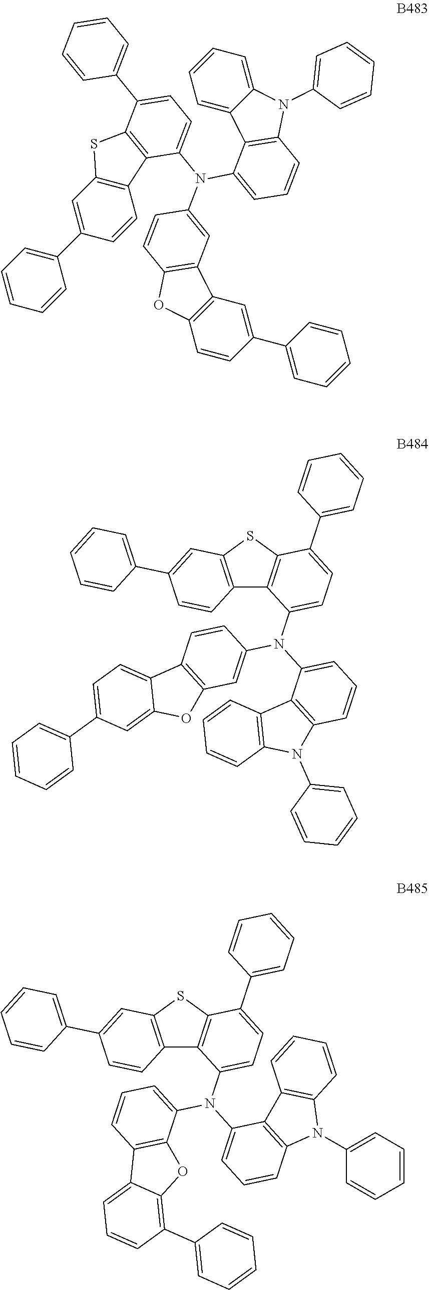 [SMILES: c1ccc(-c2ccc3c(c2)sc2c(-c4ccccc4)ccc(N(c4ccc5oc6ccc(-c7ccccc7)cc6c5c4)c4cccc5c4c4ccccc4n5-c4ccccc4)c23)cc1 ; c1ccc(-c2ccc3c(c2)oc2cc(N(c4ccc(-c5ccccc5)c5sc6cc(-c7ccccc7)ccc6c45)c4cccc5c4c4ccccc4n5-c4ccccc4)ccc23)cc1 ; c1ccc(-c2ccc3c(c2)sc2c(-c4ccccc4)ccc(N(c4cccc5c4oc4c(-c6ccccc6)cccc45)c4cccc5c4c4ccccc4n5-c4ccccc4)c23)cc1]

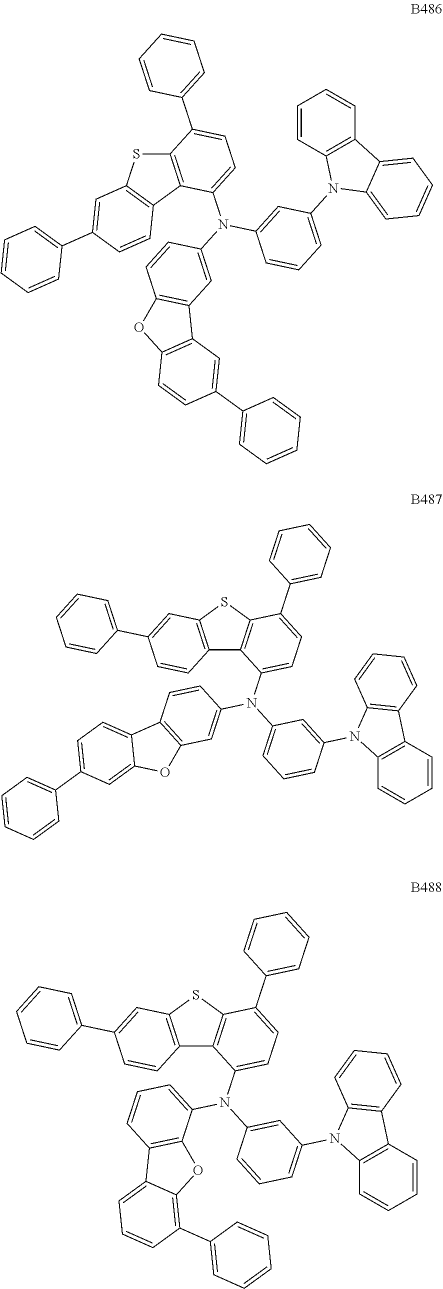 [SMILES: c1ccc(-c2ccc3c(c2)sc2c(-c4ccccc4)ccc(N(c4cccc(-n5c6ccccc6c6ccccc65)c4)c4ccc5oc6ccc(-c7ccccc7)cc6c5c4)c23)cc1 ; c1ccc(-c2ccc3c(c2)oc2cc(N(c4cccc(-n5c6ccccc6c6ccccc65)c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4cccc(-n5c6ccccc6c6ccccc65)c4)c4cccc5c4oc4c(-c6ccccc6)cccc45)c23)cc1]

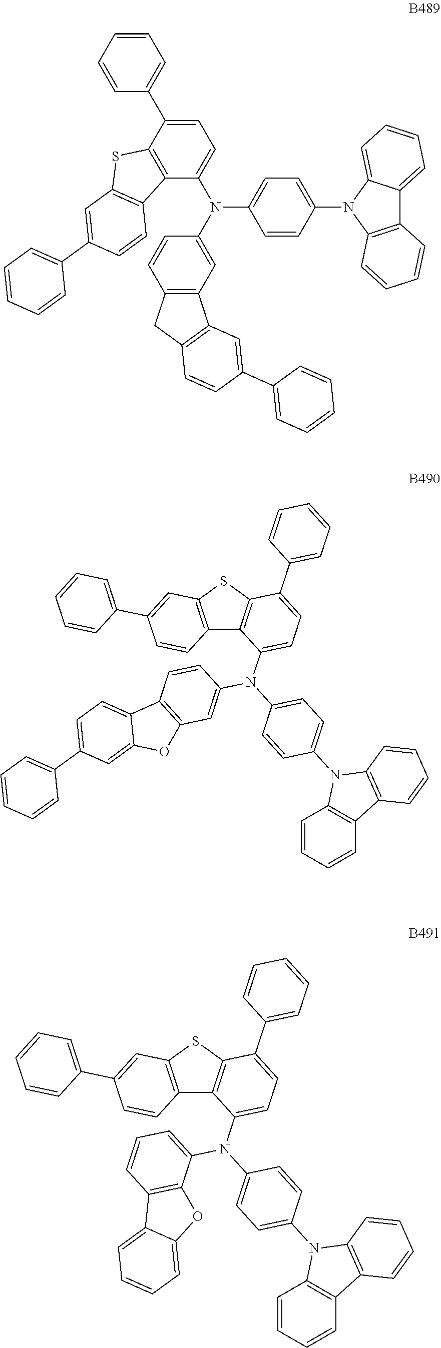 [SMILES: c1ccc(-c2ccc3c(c2)-c2cc(N(c4ccc(-n5c6ccccc6c6ccccc65)cc4)c4ccc(-c5ccccc5)c5sc6cc(-c7ccccc7)ccc6c45)ccc2C3)cc1 ; c1ccc(-c2ccc3c(c2)oc2cc(N(c4ccc(-n5c6ccccc6c6ccccc65)cc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc(-n5c6ccccc6c6ccccc65)cc4)c4cccc5c4oc4ccccc45)c23)cc1]

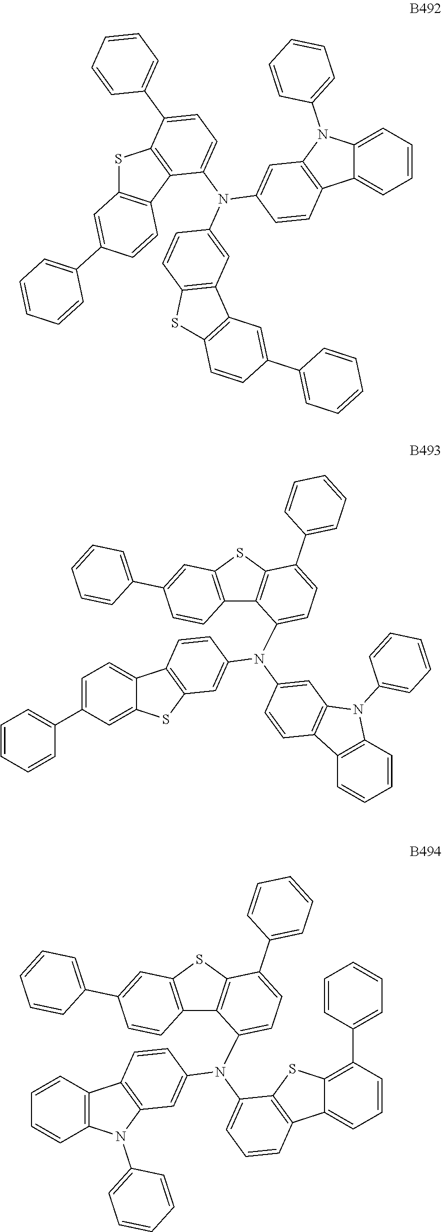 [SMILES: c1ccc(-c2ccc3c(c2)sc2c(-c4ccccc4)ccc(N(c4ccc5sc6ccc(-c7ccccc7)cc6c5c4)c4ccc5c6ccccc6n(-c6ccccc6)c5c4)c23)cc1 ; c1ccc(-c2ccc3c(c2)sc2cc(N(c4ccc5c6ccccc6n(-c6ccccc6)c5c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c6ccccc6n(-c6ccccc6)c5c4)c4cccc5c4sc4c(-c6ccccc6)cccc45)c23)cc1]

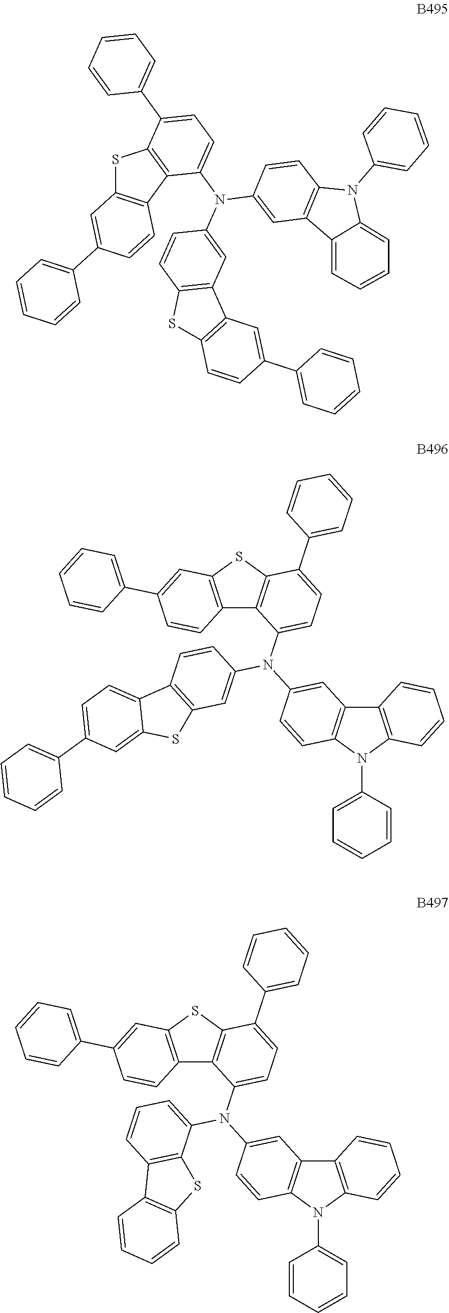 [SMILES: c1ccc(-c2ccc3c(c2)sc2c(-c4ccccc4)ccc(N(c4ccc5sc6ccc(-c7ccccc7)cc6c5c4)c4ccc5c(c4)c4ccccc4n5-c4ccccc4)c23)cc1 ; c1ccc(-c2ccc3c(c2)sc2cc(N(c4ccc5c(c4)c4ccccc4n5-c4ccccc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc5c(c4)c4ccccc4n5-c4ccccc4)c4cccc5c4sc4ccccc45)c23)cc1]

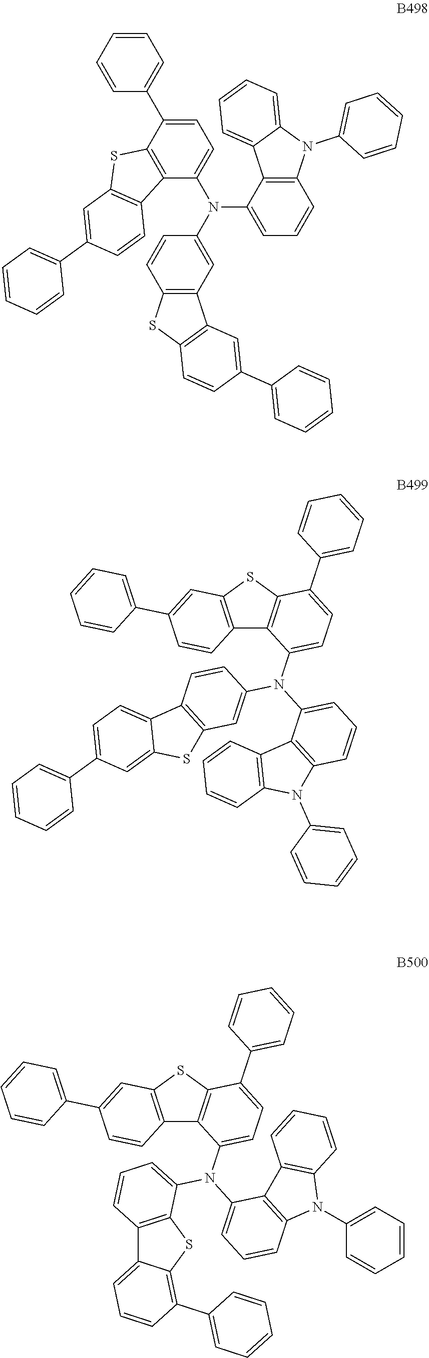 [SMILES: c1ccc(-c2ccc3c(c2)sc2c(-c4ccccc4)ccc(N(c4ccc5sc6ccc(-c7ccccc7)cc6c5c4)c4cccc5c4c4ccccc4n5-c4ccccc4)c23)cc1 ; c1ccc(-c2ccc3c(c2)sc2cc(N(c4ccc(-c5ccccc5)c5sc6cc(-c7ccccc7)ccc6c45)c4cccc5c4c4ccccc4n5-c4ccccc4)ccc23)cc1 ; c1ccc(-c2ccc3c(c2)sc2c(-c4ccccc4)ccc(N(c4cccc5c4sc4c(-c6ccccc6)cccc45)c4cccc5c4c4ccccc4n5-c4ccccc4)c23)cc1]

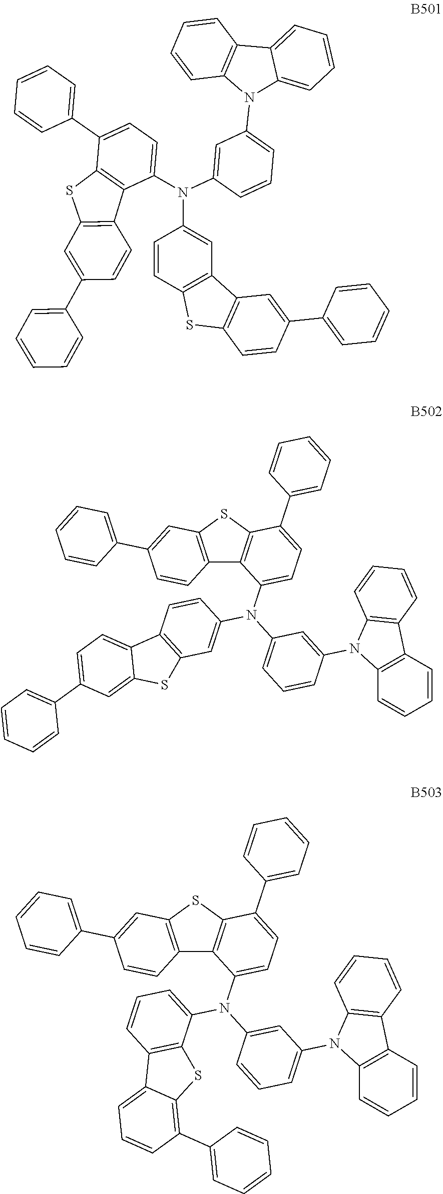 [SMILES: c1ccc(-c2ccc3c(c2)sc2c(-c4ccccc4)ccc(N(c4cccc(-n5c6ccccc6c6ccccc65)c4)c4ccc5sc6ccc(-c7ccccc7)cc6c5c4)c23)cc1 ; c1ccc(-c2ccc3c(c2)sc2cc(N(c4cccc(-n5c6ccccc6c6ccccc65)c4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4cccc(-n5c6ccccc6c6ccccc65)c4)c4cccc5c4sc4c(-c6ccccc6)cccc45)c23)cc1]

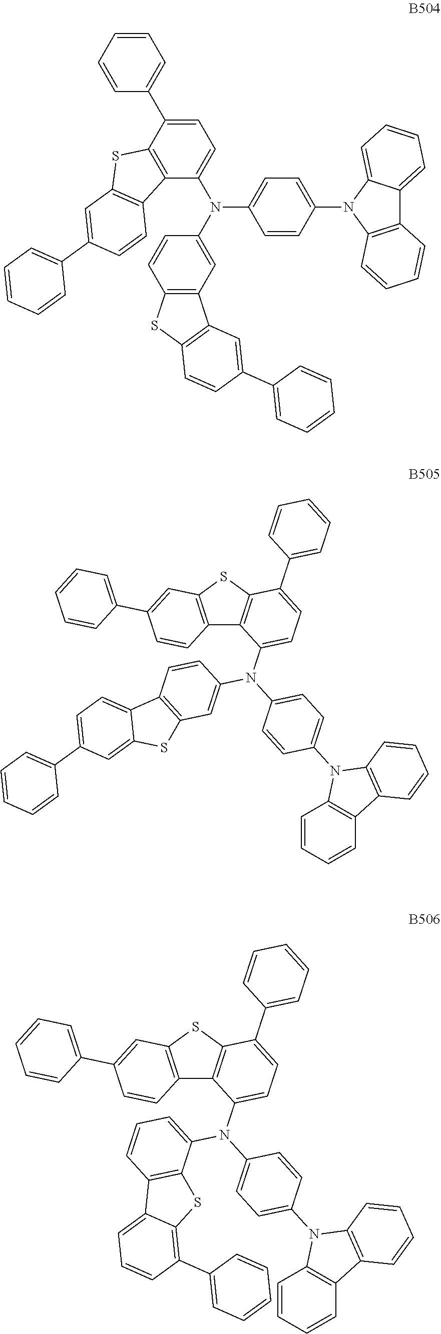 [SMILES: c1ccc(-c2ccc3c(c2)sc2c(-c4ccccc4)ccc(N(c4ccc(-n5c6ccccc6c6ccccc65)cc4)c4ccc5sc6ccc(-c7ccccc7)cc6c5c4)c23)cc1 ; c1ccc(-c2ccc3c(c2)sc2cc(N(c4ccc(-n5c6ccccc6c6ccccc65)cc4)c4ccc(-c5ccccc5)c5sc6cc(-c7ccccc7)ccc6c45)ccc23)cc1 ; c1ccc(-c2ccc3c(c2)sc2c(-c4ccccc4)ccc(N(c4ccc(-n5c6ccccc6c6ccccc65)cc4)c4cccc5c4sc4c(-c6ccccc6)cccc45)c23)cc1]

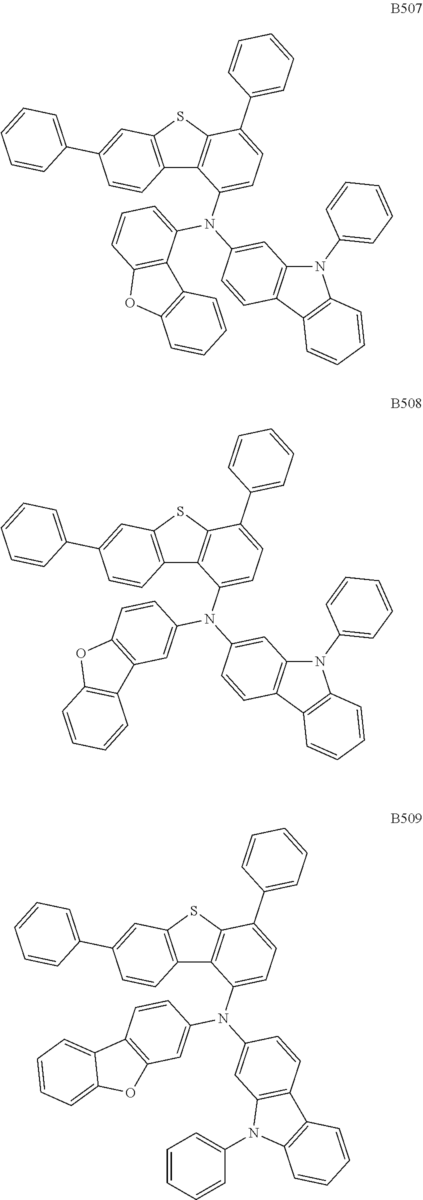 [SMILES: c1ccc(-c2ccc3c(c2)sc2c(-c4ccccc4)ccc(N(c4ccc5c6ccccc6n(-c6ccccc6)c5c4)c4cccc5oc6ccccc6c45)c23)cc1 ; c1ccc(-c2ccc3c(c2)sc2c(-c4ccccc4)ccc(N(c4ccc5oc6ccccc6c5c4)c4ccc5c6ccccc6n(-c6ccccc6)c5c4)c23)cc1 ; c1ccc(-c2ccc3c(c2)sc2c(-c4ccccc4)ccc(N(c4ccc5c(c4)oc4ccccc45)c4ccc5c6ccccc6n(-c6ccccc6)c5c4)c23)cc1]

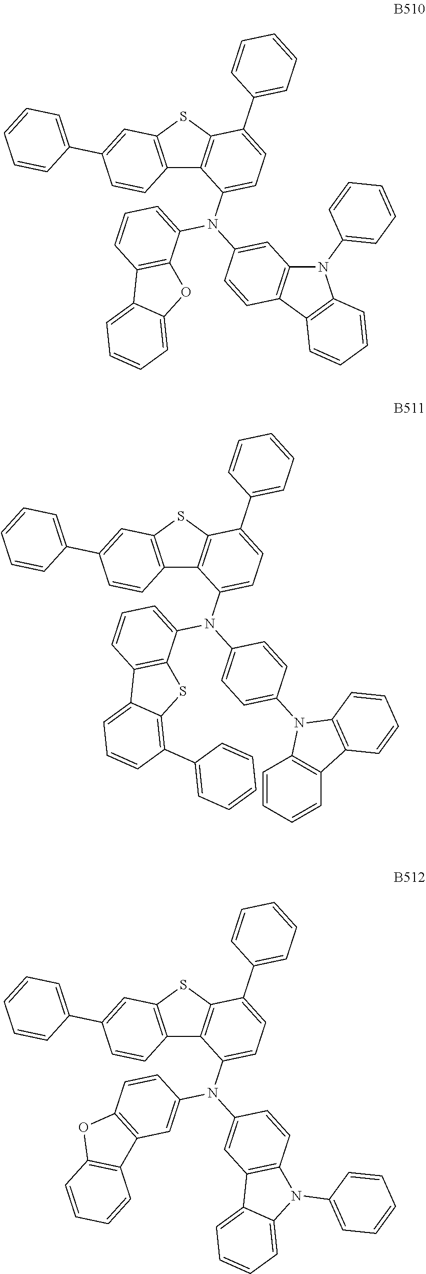 [SMILES: c1ccc(-c2ccc3c(c2)sc2c(-c4ccccc4)ccc(N(c4ccc5c6ccccc6n(-c6ccccc6)c5c4)c4cccc5c4oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5c4sc4c(-c6ccccc6)cccc45)c23)cc1 ; c1ccc(-c2ccc3c(c2)sc2c(-c4ccccc4)ccc(N(c4ccc5oc6ccccc6c5c4)c4ccc5c(c4)c4ccccc4n5-c4ccccc4)c23)cc1]

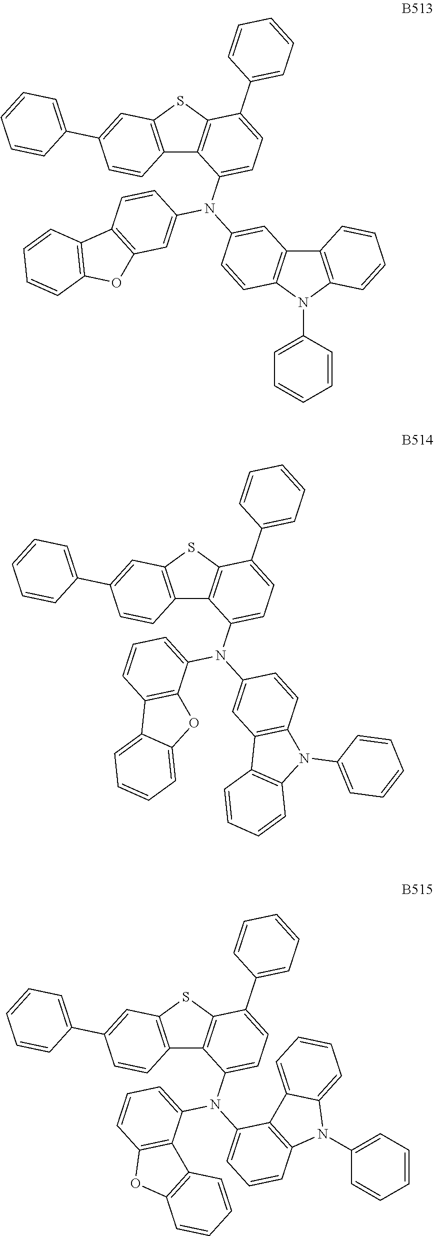 [SMILES: c1ccc(-c2ccc3c(c2)sc2c(-c4ccccc4)ccc(N(c4ccc5c(c4)oc4ccccc45)c4ccc5c(c4)c4ccccc4n5-c4ccccc4)c23)cc1 ; c1ccc(-c2ccc3c(c2)sc2c(-c4ccccc4)ccc(N(c4ccc5c(c4)c4ccccc4n5-c4ccccc4)c4cccc5c4oc4ccccc45)c23)cc1 ; c1ccc(-c2ccc3c(c2)sc2c(-c4ccccc4)ccc(N(c4cccc5oc6ccccc6c45)c4cccc5c4c4ccccc4n5-c4ccccc4)c23)cc1]

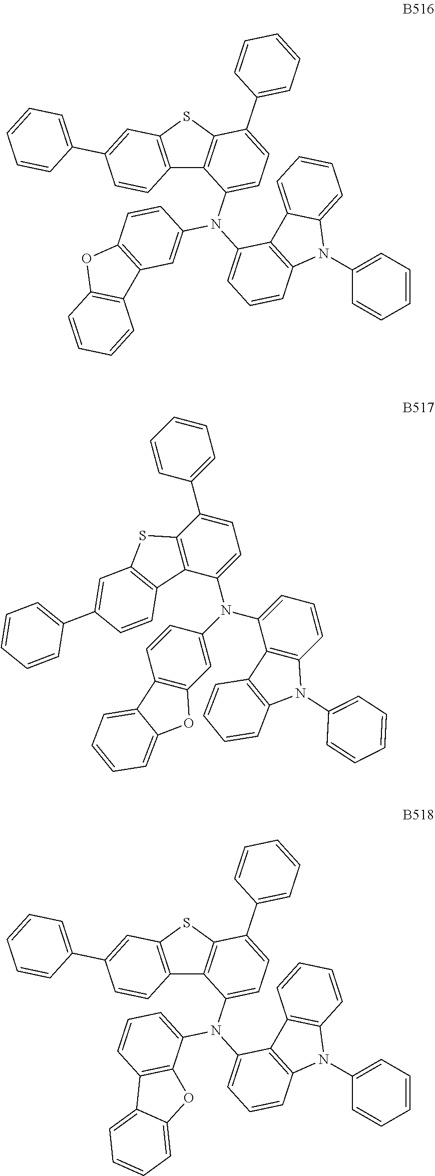 [SMILES: c1ccc(-c2ccc3c(c2)sc2c(-c4ccccc4)ccc(N(c4ccc5oc6ccccc6c5c4)c4cccc5c4c4ccccc4n5-c4ccccc4)c23)cc1 ; c1ccc(-c2ccc3c(c2)sc2c(-c4ccccc4)ccc(N(c4ccc5c(c4)oc4ccccc45)c4cccc5c4c4ccccc4n5-c4ccccc4)c23)cc1 ; c1ccc(-c2ccc3c(c2)sc2c(-c4ccccc4)ccc(N(c4cccc5c4oc4ccccc45)c4cccc5c4c4ccccc4n5-c4ccccc4)c23)cc1]

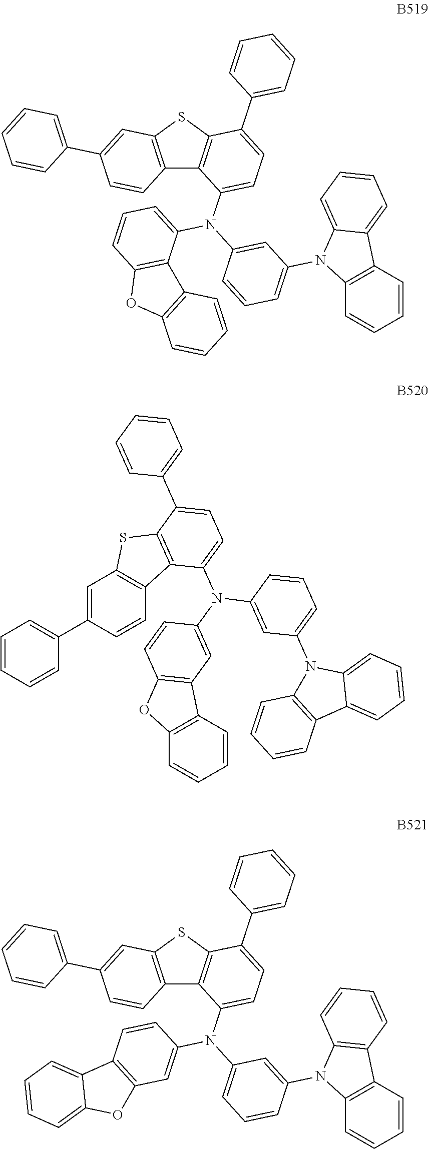 [SMILES: c1ccc(-c2ccc3c(c2)sc2c(-c4ccccc4)ccc(N(c4cccc(-n5c6ccccc6c6ccccc65)c4)c4cccc5oc6ccccc6c45)c23)cc1 ; c1ccc(-c2ccc3c(c2)sc2c(-c4ccccc4)ccc(N(c4cccc(-n5c6ccccc6c6ccccc65)c4)c4ccc5oc6ccccc6c5c4)c23)cc1 ; c1ccc(-c2ccc3c(c2)sc2c(-c4ccccc4)ccc(N(c4cccc(-n5c6ccccc6c6ccccc65)c4)c4ccc5c(c4)oc4ccccc45)c23)cc1]

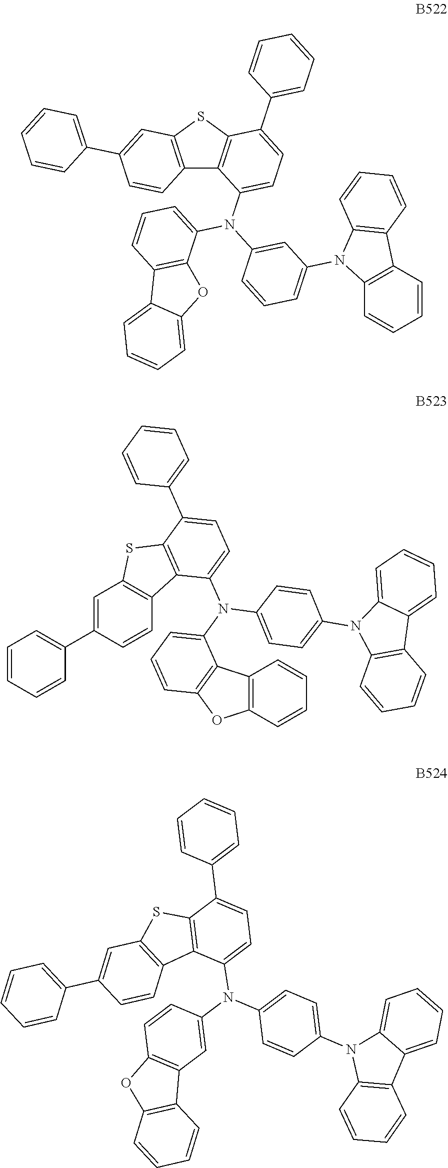 [SMILES: c1ccc(-c2ccc3c(c2)sc2c(-c4ccccc4)ccc(N(c4cccc(-n5c6ccccc6c6ccccc65)c4)c4cccc5c4oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5oc6ccccc6c45)c23)cc1 ; c1ccc(-c2ccc3c(c2)sc2c(-c4ccccc4)ccc(N(c4ccc(-n5c6ccccc6c6ccccc65)cc4)c4ccc5oc6ccccc6c5c4)c23)cc1]

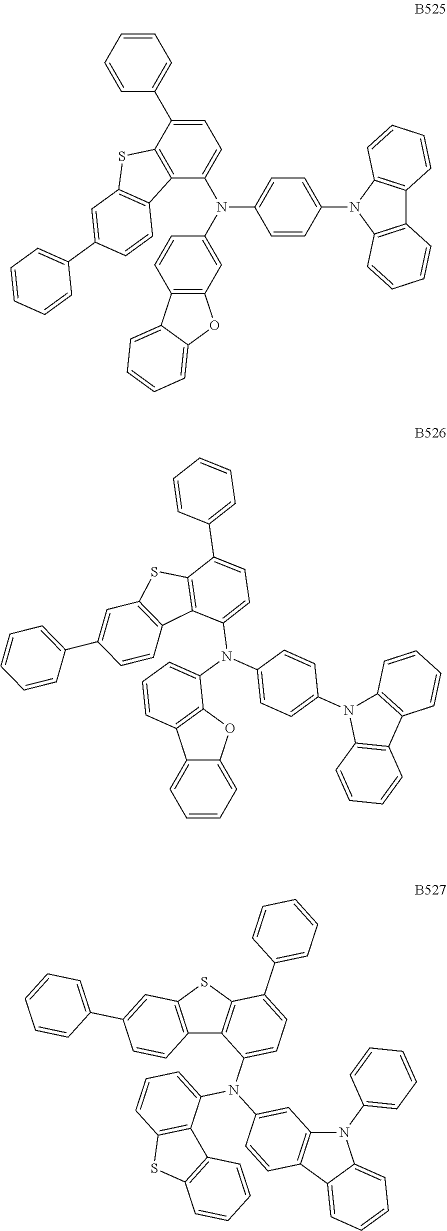 [SMILES: c1ccc(-c2ccc3c(c2)sc2c(-c4ccccc4)ccc(N(c4ccc(-n5c6ccccc6c6ccccc65)cc4)c4ccc5c(c4)oc4ccccc45)c23)cc1 ; c1ccc(-c2ccc3c(c2)sc2c(-c4ccccc4)ccc(N(c4ccc(-n5c6ccccc6c6ccccc65)cc4)c4cccc5c4oc4ccccc45)c23)cc1 ; c1ccc(-c2ccc3c(c2)sc2c(-c4ccccc4)ccc(N(c4ccc5c6ccccc6n(-c6ccccc6)c5c4)c4cccc5sc6ccccc6c45)c23)cc1]

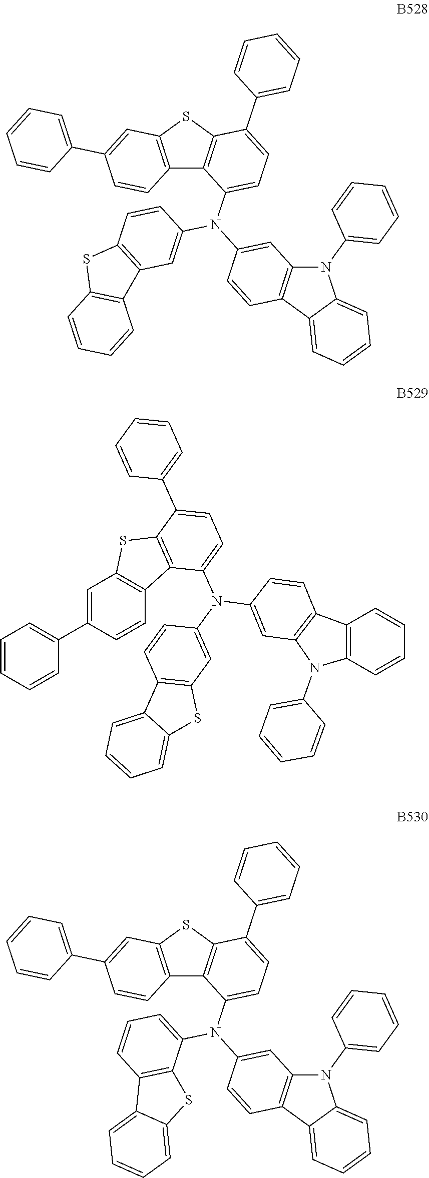 [SMILES: c1ccc(-c2ccc3c(c2)sc2c(-c4ccccc4)ccc(N(c4ccc5sc6ccccc6c5c4)c4ccc5c6ccccc6n(-c6ccccc6)c5c4)c23)cc1 ; c1ccc(-c2ccc3c(c2)sc2c(-c4ccccc4)ccc(N(c4ccc5c(c4)sc4ccccc45)c4ccc5c6ccccc6n(-c6ccccc6)c5c4)c23)cc1 ; c1ccc(-c2ccc3c(c2)sc2c(-c4ccccc4)ccc(N(c4ccc5c6ccccc6n(-c6ccccc6)c5c4)c4cccc5c4sc4ccccc45)c23)cc1]

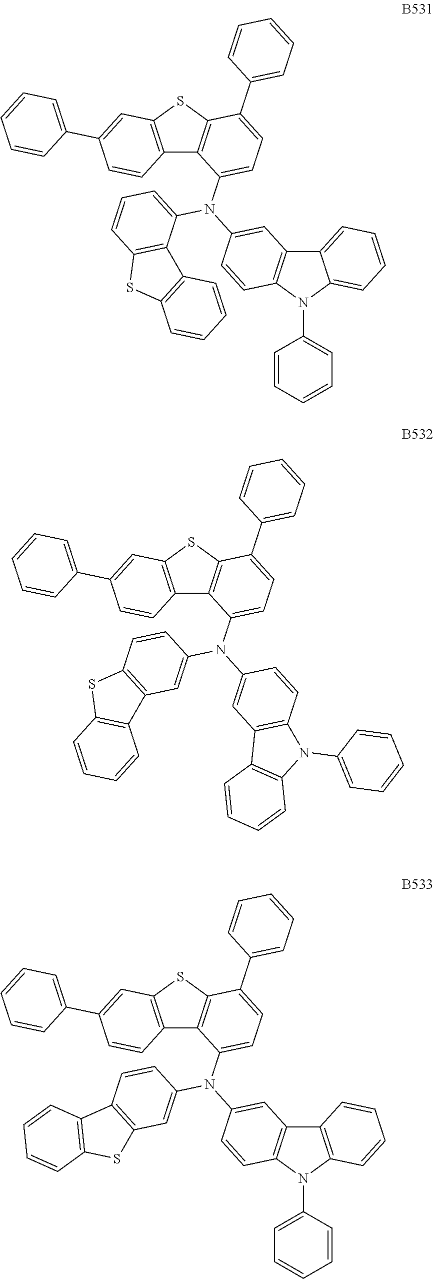 [SMILES: c1ccc(-c2ccc3c(c2)sc2c(-c4ccccc4)ccc(N(c4ccc5c(c4)c4ccccc4n5-c4ccccc4)c4cccc5sc6ccccc6c45)c23)cc1 ; c1ccc(-c2ccc3c(c2)sc2c(-c4ccccc4)ccc(N(c4ccc5sc6ccccc6c5c4)c4ccc5c(c4)c4ccccc4n5-c4ccccc4)c23)cc1 ; c1ccc(-c2ccc3c(c2)sc2c(-c4ccccc4)ccc(N(c4ccc5c(c4)sc4ccccc45)c4ccc5c(c4)c4ccccc4n5-c4ccccc4)c23)cc1]

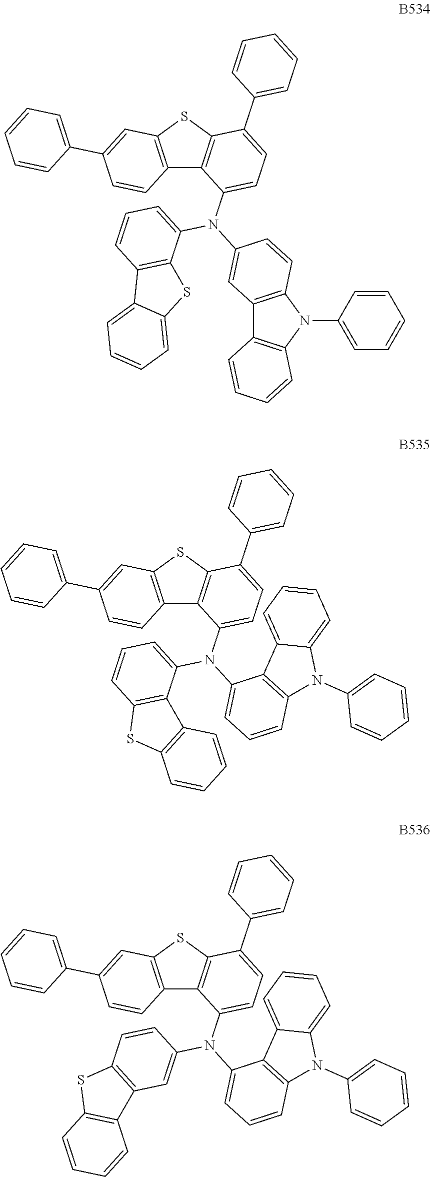 [SMILES: c1ccc(-c2ccc3c(c2)sc2c(-c4ccccc4)ccc(N(c4ccc5c(c4)c4ccccc4n5-c4ccccc4)c4cccc5c4sc4ccccc45)c23)cc1 ; c1ccc(-c2ccc3c(c2)sc2c(-c4ccccc4)ccc(N(c4cccc5sc6ccccc6c45)c4cccc5c4c4ccccc4n5-c4ccccc4)c23)cc1 ; c1ccc(-c2ccc3c(c2)sc2c(-c4ccccc4)ccc(N(c4ccc5sc6ccccc6c5c4)c4cccc5c4c4ccccc4n5-c4ccccc4)c23)cc1]

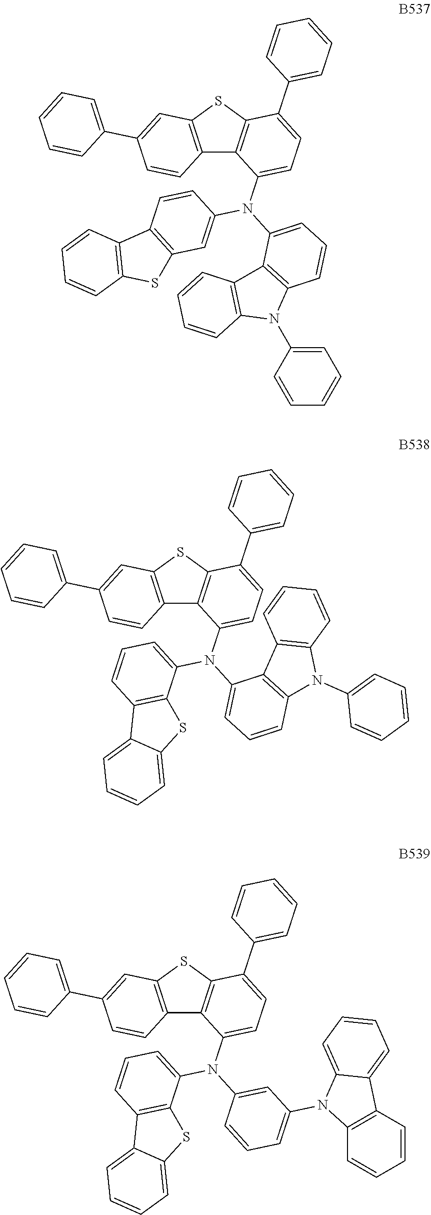 [SMILES: c1ccc(-c2ccc3c(c2)sc2c(-c4ccccc4)ccc(N(c4ccc5c(c4)sc4ccccc45)c4cccc5c4c4ccccc4n5-c4ccccc4)c23)cc1 ; c1ccc(-c2ccc3c(c2)sc2c(-c4ccccc4)ccc(N(c4cccc5c4sc4ccccc45)c4cccc5c4c4ccccc4n5-c4ccccc4)c23)cc1 ; c1ccc(-c2ccc3c(c2)sc2c(-c4ccccc4)ccc(N(c4cccc(-n5c6ccccc6c6ccccc65)c4)c4cccc5c4sc4ccccc45)c23)cc1]

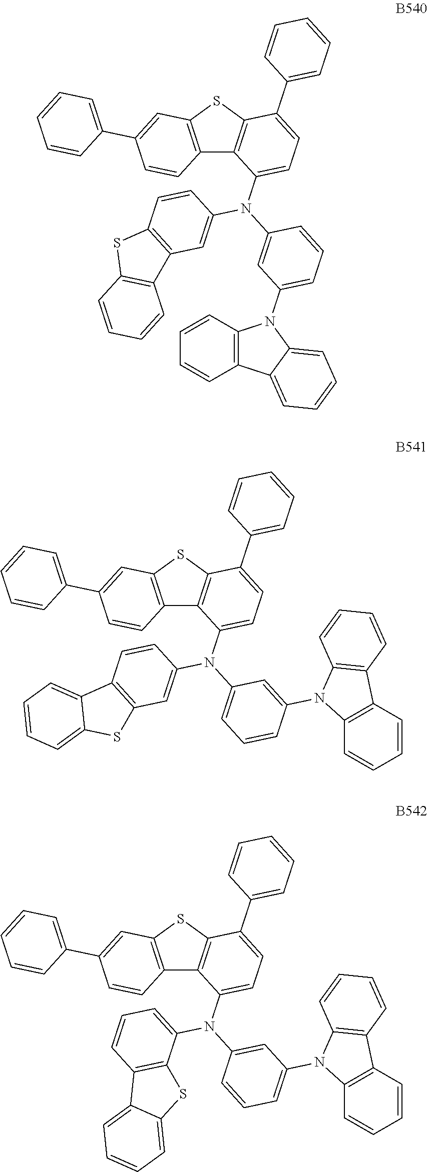 [SMILES: c1ccc(-c2ccc3c(c2)sc2c(-c4ccccc4)ccc(N(c4cccc(-n5c6ccccc6c6ccccc65)c4)c4ccc5sc6ccccc6c5c4)c23)cc1 ; c1ccc(-c2ccc3c(c2)sc2c(-c4ccccc4)ccc(N(c4cccc(-n5c6ccccc6c6ccccc65)c4)c4ccc5c(c4)sc4ccccc45)c23)cc1 ; c1ccc(-c2ccc3c(c2)sc2c(-c4ccccc4)ccc(N(c4cccc(-n5c6ccccc6c6ccccc65)c4)c4cccc5c4sc4ccccc45)c23)cc1]

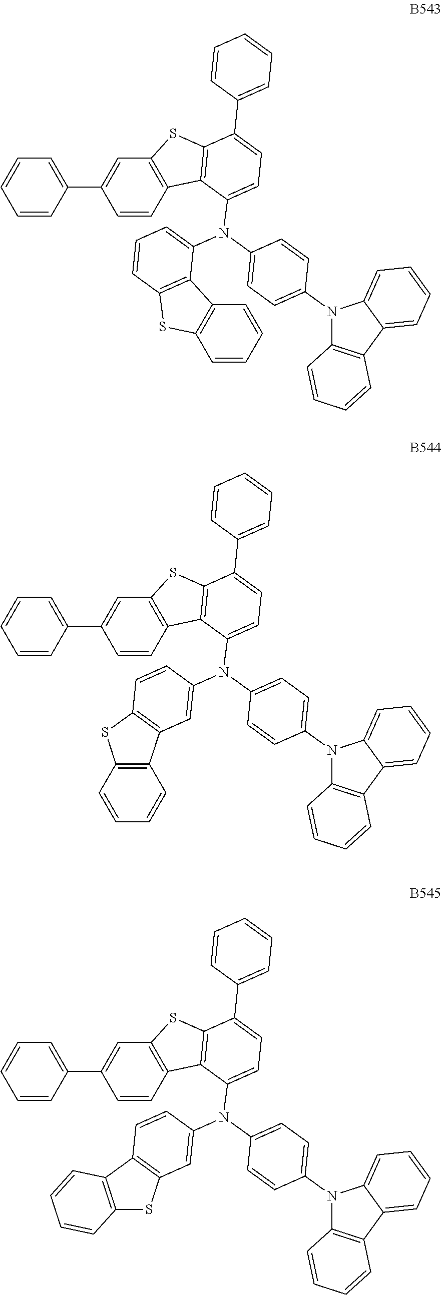 [SMILES: c1ccc(-c2ccc3c(c2)sc2c(-c4ccccc4)ccc(N(c4ccc(-n5c6ccccc6c6ccccc65)cc4)c4cccc5sc6ccccc6c45)c23)cc1 ; c1ccc(-c2ccc3c(c2)sc2c(-c4ccccc4)ccc(N(c4ccc(-n5c6ccccc6c6ccccc65)cc4)c4ccc5sc6ccccc6c5c4)c23)cc1 ; c1ccc(-c2ccc3c(c2)sc2c(-c4ccccc4)ccc(N(c4ccc(-n5c6ccccc6c6ccccc65)cc4)c4ccc5c(c4)sc4ccccc45)c23)cc1]

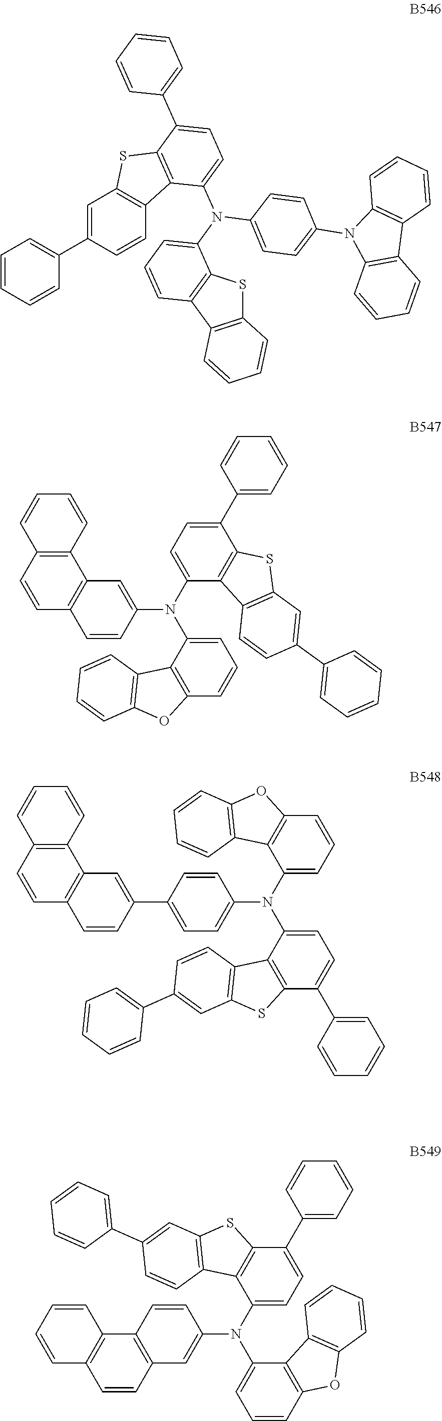 [SMILES: c1ccc(-c2ccc3c(c2)sc2c(-c4ccccc4)ccc(N(c4ccc(-n5c6ccccc6c6ccccc65)cc4)c4cccc5c4sc4ccccc45)c23)cc1 ; c1ccc(-c2ccc3c(c2)sc2c(-c4ccccc4)ccc(N(c4ccc5ccc6ccccc6c5c4)c4cccc5oc6ccccc6c45)c23)cc1 ; c1ccc(-c2ccc3c(c2)sc2c(-c4ccccc4)ccc(N(c4ccc(-c5ccc6ccc7ccccc7c6c5)cc4)c4cccc5oc6ccccc6c45)c23)cc1 ; c1ccc(-c2ccc3c(c2)sc2c(-c4ccccc4)ccc(N(c4ccc5c(ccc6ccccc65)c4)c4cccc5oc6ccccc6c45)c23)cc1]

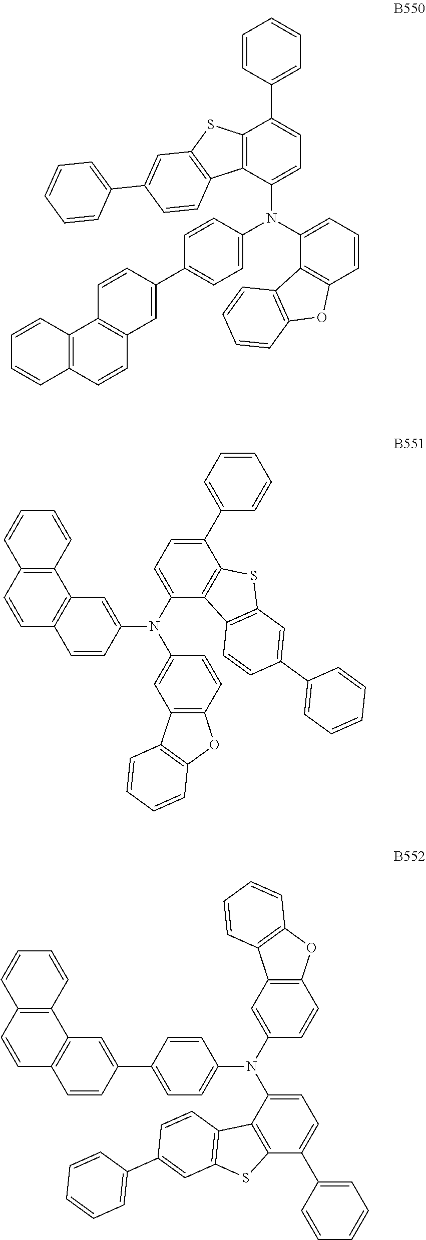 [SMILES: c1ccc(-c2ccc3c(c2)sc2c(-c4ccccc4)ccc(N(c4ccc(-c5ccc6c(ccc7ccccc76)c5)cc4)c4cccc5oc6ccccc6c45)c23)cc1 ; c1ccc(-c2ccc3c(c2)sc2c(-c4ccccc4)ccc(N(c4ccc5ccc6ccccc6c5c4)c4ccc5oc6ccccc6c5c4)c23)cc1 ; c1ccc(-c2ccc3c(c2)sc2c(-c4ccccc4)ccc(N(c4ccc(-c5ccc6ccc7ccccc7c6c5)cc4)c4ccc5oc6ccccc6c5c4)c23)cc1]

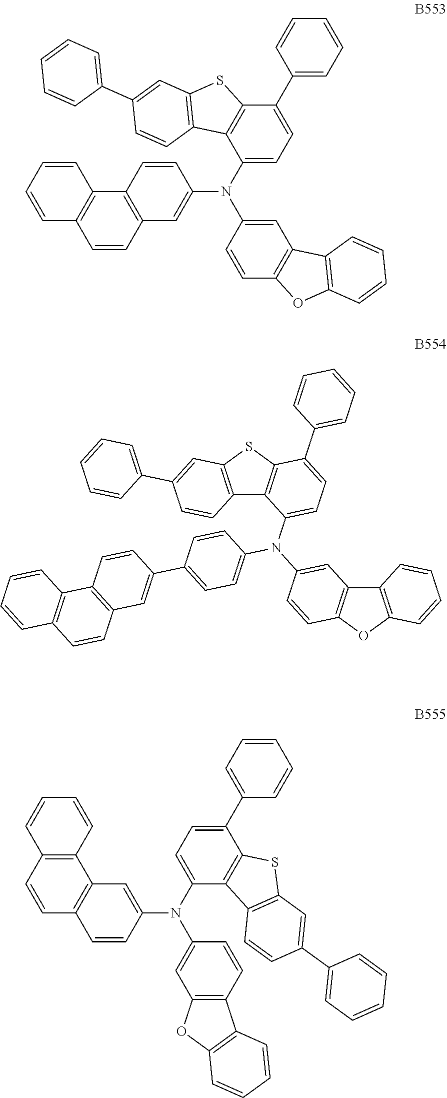 [SMILES: c1ccc(-c2ccc3c(c2)sc2c(-c4ccccc4)ccc(N(c4ccc5c(ccc6ccccc65)c4)c4ccc5oc6ccccc6c5c4)c23)cc1 ; c1ccc(-c2ccc3c(c2)sc2c(-c4ccccc4)ccc(N(c4ccc(-c5ccc6c(ccc7ccccc76)c5)cc4)c4ccc5oc6ccccc6c5c4)c23)cc1 ; c1ccc(-c2ccc3c(c2)sc2c(-c4ccccc4)ccc(N(c4ccc5c(c4)oc4ccccc45)c4ccc5ccc6ccccc6c5c4)c23)cc1]

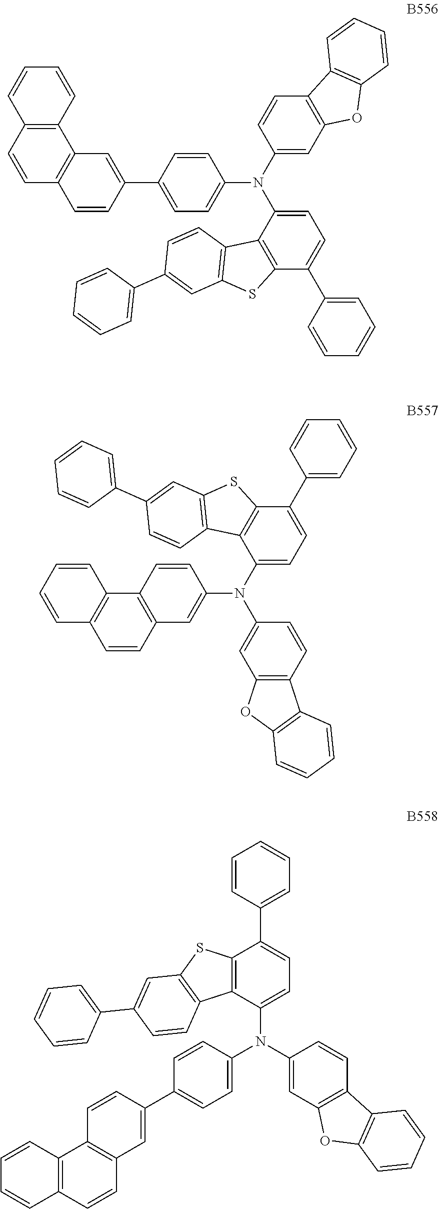 [SMILES: c1ccc(-c2ccc3c(c2)sc2c(-c4ccccc4)ccc(N(c4ccc(-c5ccc6ccc7ccccc7c6c5)cc4)c4ccc5c(c4)oc4ccccc45)c23)cc1 ; c1ccc(-c2ccc3c(c2)sc2c(-c4ccccc4)ccc(N(c4ccc5c(ccc6ccccc65)c4)c4ccc5c(c4)oc4ccccc45)c23)cc1 ; c1ccc(-c2ccc3c(c2)sc2c(-c4ccccc4)ccc(N(c4ccc(-c5ccc6c(ccc7ccccc76)c5)cc4)c4ccc5c(c4)oc4ccccc45)c23)cc1]

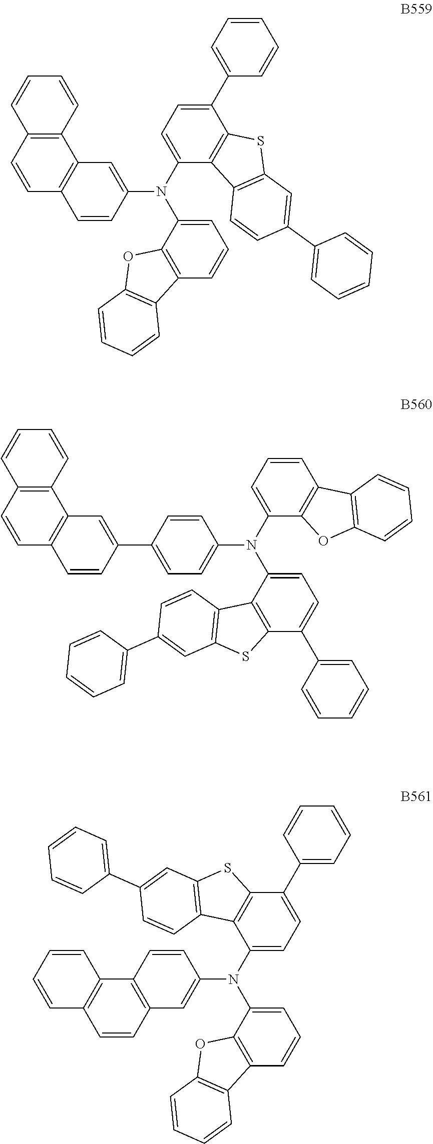 [SMILES: c1ccc(-c2ccc3c(c2)sc2c(-c4ccccc4)ccc(N(c4ccc5ccc6ccccc6c5c4)c4cccc5c4oc4ccccc45)c23)cc1 ; c1ccc(-c2ccc3c(c2)sc2c(-c4ccccc4)ccc(N(c4ccc(-c5ccc6ccc7ccccc7c6c5)cc4)c4cccc5c4oc4ccccc45)c23)cc1 ; c1ccc(-c2ccc3c(c2)sc2c(-c4ccccc4)ccc(N(c4ccc5c(ccc6ccccc65)c4)c4cccc5c4oc4ccccc45)c23)cc1]

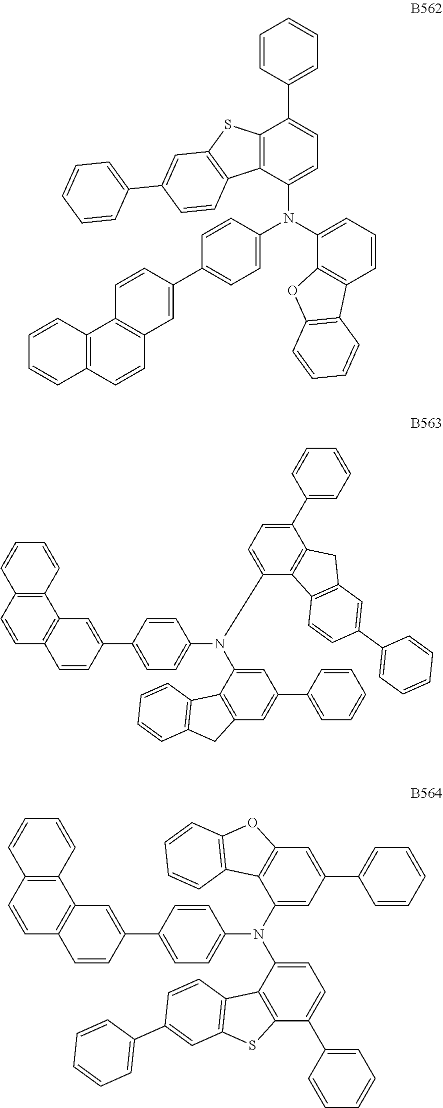 [SMILES: c1ccc(-c2ccc3c(c2)sc2c(-c4ccccc4)ccc(N(c4ccc(-c5ccc6c(ccc7ccccc76)c5)cc4)c4cccc5c4oc4ccccc45)c23)cc1 ; c1ccc(-c2ccc3c(c2)Cc2c(-c4ccccc4)ccc(N(c4ccc(-c5ccc6ccc7ccccc7c6c5)cc4)c4cc(-c5ccccc5)cc5c4-c4ccccc4C5)c2-3)cc1 ; c1ccc(-c2cc(N(c3ccc(-c4ccc5ccc6ccccc6c5c4)cc3)c3ccc(-c4ccccc4)c4sc5cc(-c6ccccc6)ccc5c34)c3c(c2)oc2ccccc23)cc1]

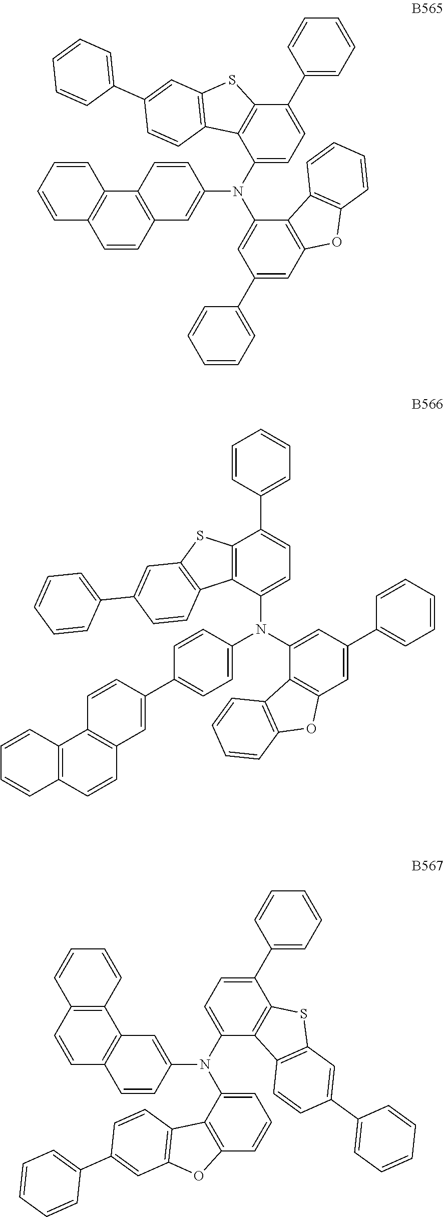 [SMILES: c1ccc(-c2cc(N(c3ccc4c(ccc5ccccc54)c3)c3ccc(-c4ccccc4)c4sc5cc(-c6ccccc6)ccc5c34)c3c(c2)oc2ccccc23)cc1 ; c1ccc(-c2cc(N(c3ccc(-c4ccc5c(ccc6ccccc65)c4)cc3)c3ccc(-c4ccccc4)c4sc5cc(-c6ccccc6)ccc5c34)c3c(c2)oc2ccccc23)cc1 ; c1ccc(-c2ccc3c(c2)oc2cccc(N(c4ccc5ccc6ccccc6c5c4)c4ccc(-c5ccccc5)c5sc6cc(-c7ccccc7)ccc6c45)c23)cc1]

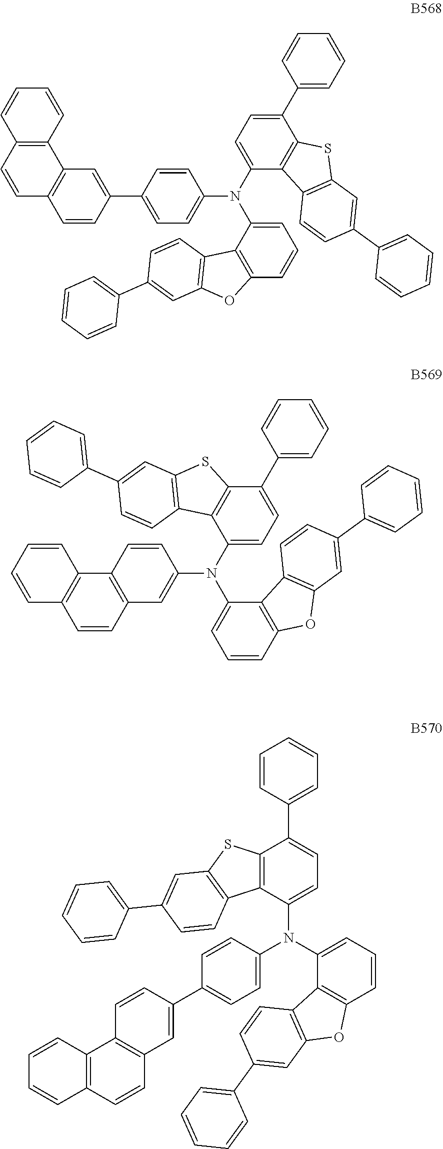 [SMILES: c1ccc(-c2ccc3c(c2)oc2cccc(N(c4ccc(-c5ccc6ccc7ccccc7c6c5)cc4)c4ccc(-c5ccccc5)c5sc6cc(-c7ccccc7)ccc6c45)c23)cc1 ; c1ccc(-c2ccc3c(c2)oc2cccc(N(c4ccc5c(ccc6ccccc65)c4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6c(ccc7ccccc76)c5)cc4)c4ccc(-c5ccccc5)c5sc6cc(-c7ccccc7)ccc6c45)c23)cc1]

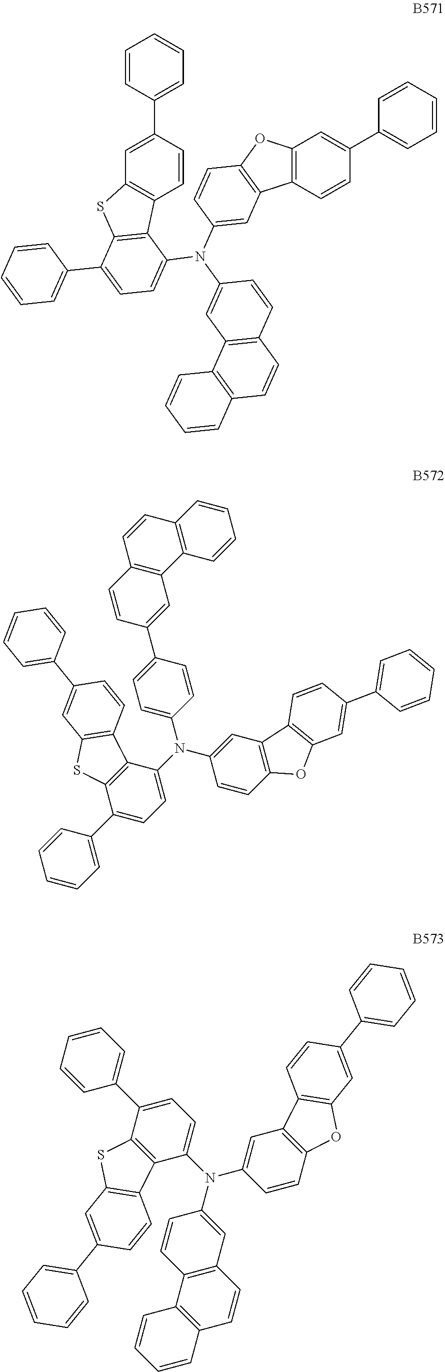 [SMILES: c1ccc(-c2ccc3c(c2)oc2ccc(N(c4ccc5ccc6ccccc6c5c4)c4ccc(-c5ccccc5)c5sc6cc(-c7ccccc7)ccc6c45)cc23)cc1 ; c1ccc(-c2ccc3c(c2)oc2ccc(N(c4ccc(-c5ccc6ccc7ccccc7c6c5)cc4)c4ccc(-c5ccccc5)c5sc6cc(-c7ccccc7)ccc6c45)cc23)cc1 ; c1ccc(-c2ccc3c(c2)oc2ccc(N(c4ccc5c(ccc6ccccc65)c4)c4ccc(-c5ccccc5)c5sc6cc(-c7ccccc7)ccc6c45)cc23)cc1]

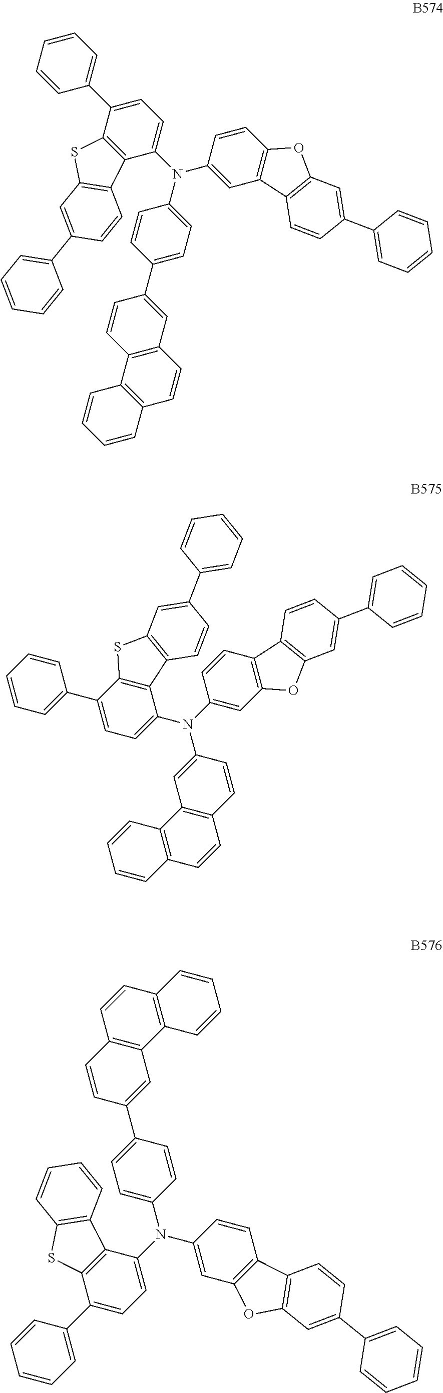 [SMILES: c1ccc(-c2ccc3c(c2)oc2ccc(N(c4ccc(-c5ccc6c(ccc7ccccc76)c5)cc4)c4ccc(-c5ccccc5)c5sc6cc(-c7ccccc7)ccc6c45)cc23)cc1 ; c1ccc(-c2ccc3c(c2)oc2cc(N(c4ccc5ccc6ccccc6c5c4)c4ccc(-c5ccccc5)c5sc6cc(-c7ccccc7)ccc6c45)ccc23)cc1 ; c1ccc(-c2ccc3c(c2)oc2cc(N(c4ccc(-c5ccc6ccc7ccccc7c6c5)cc4)c4ccc(-c5ccccc5)c5sc6ccccc6c45)ccc23)cc1]

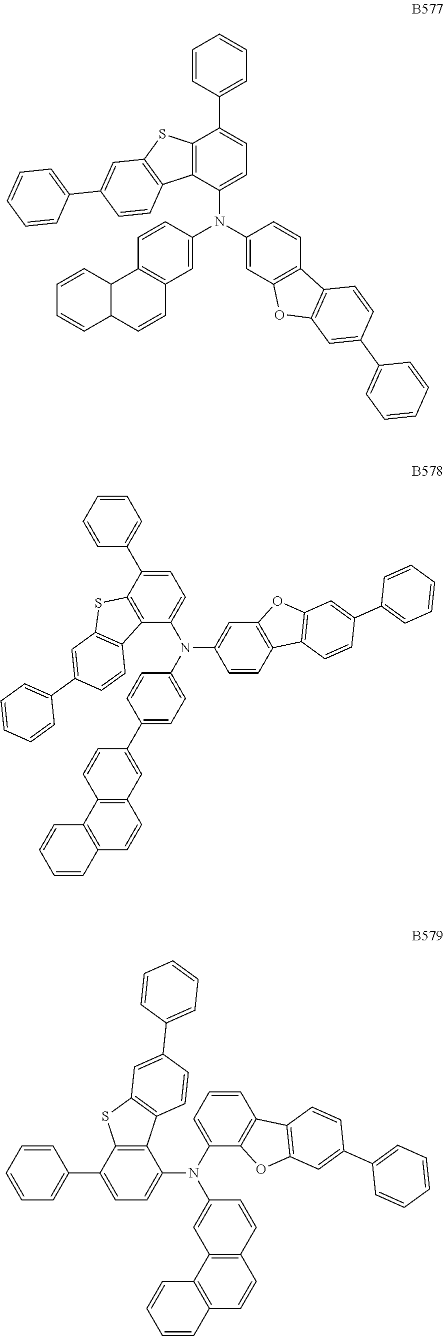

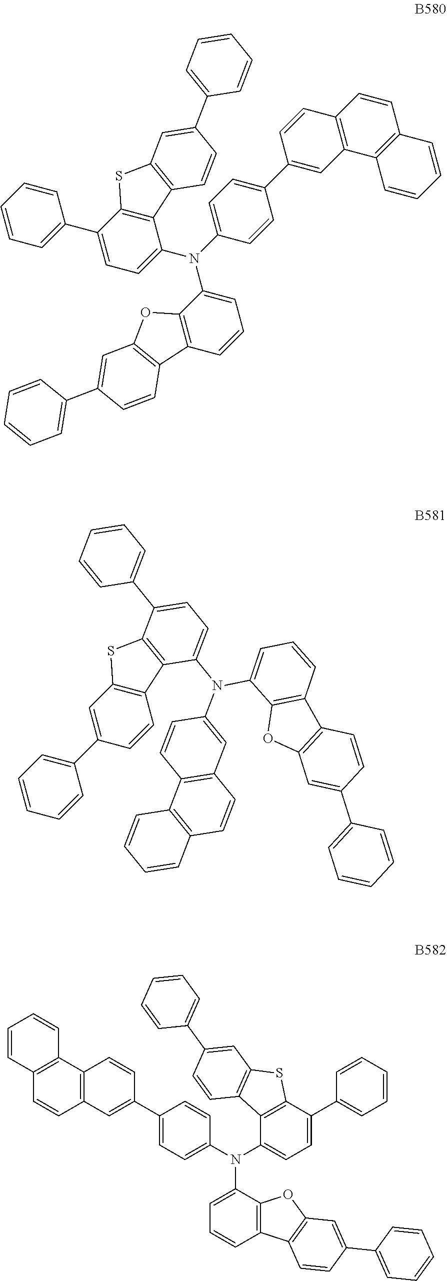 [SMILES: c1ccc(-c2ccc3c(c2)oc2c(N(c4ccc(-c5ccc6ccc7ccccc7c6c5)cc4)c4ccc(-c5ccccc5)c5sc6cc(-c7ccccc7)ccc6c45)cccc23)cc1 ; c1ccc(-c2ccc3c(c2)oc2c(N(c4ccc5c(ccc6ccccc65)c4)c4ccc(-c5ccccc5)c5sc6cc(-c7ccccc7)ccc6c45)cccc23)cc1 ; c1ccc(-c2ccc3c(c2)oc2c(N(c4ccc(-c5ccc6c(ccc7ccccc76)c5)cc4)c4ccc(-c5ccccc5)c5sc6cc(-c7ccccc7)ccc6c45)cccc23)cc1]

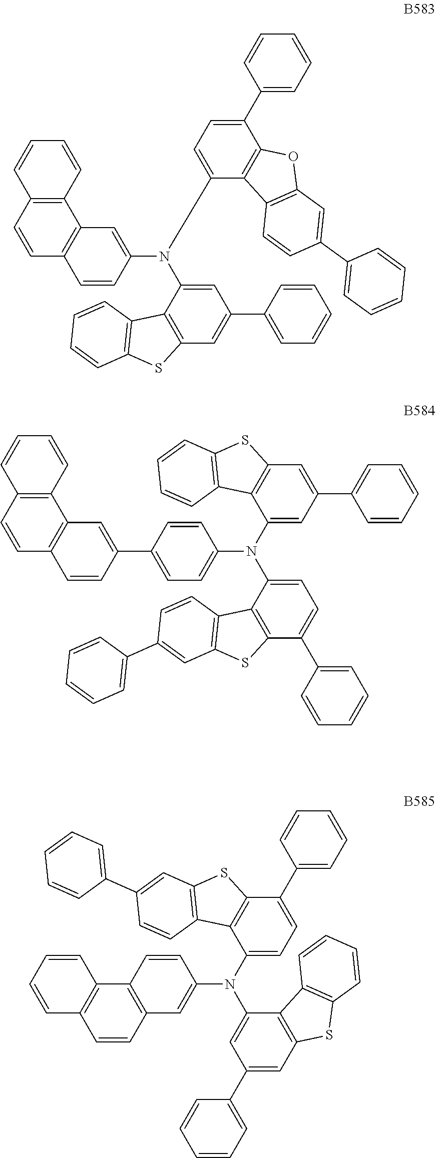 [SMILES: c1ccc(-c2ccc3c(c2)oc2c(-c4ccccc4)ccc(N(c4ccc5ccc6ccccc6c5c4)c4cc(-c5ccccc5)cc5sc6ccccc6c45)c23)cc1 ; c1ccc(-c2ccc3c(c2)sc2c(-c4ccccc4)ccc(N(c4ccc(-c5ccc6ccc7ccccc7c6c5)cc4)c4cc(-c5ccccc5)cc5sc6ccccc6c45)c23)cc1 ; c1ccc(-c2ccc3c(c2)sc2c(-c4ccccc4)ccc(N(c4ccc5c(ccc6ccccc65)c4)c4cc(-c5ccccc5)cc5sc6ccccc6c45)c23)cc1]

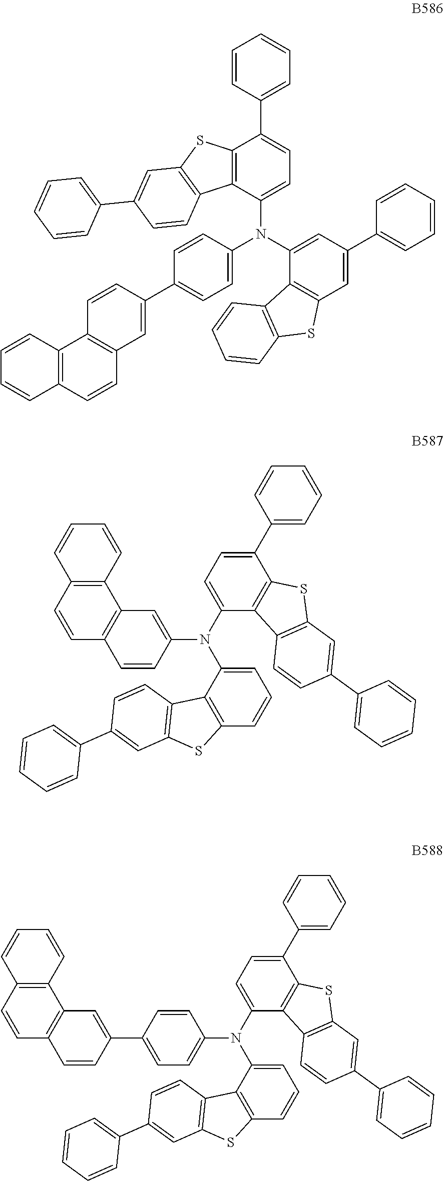 [SMILES: c1ccc(-c2ccc3c(c2)sc2c(-c4ccccc4)ccc(N(c4ccc(-c5ccc6c(ccc7ccccc76)c5)cc4)c4cc(-c5ccccc5)cc5sc6ccccc6c45)c23)cc1 ; c1ccc(-c2ccc3c(c2)sc2cccc(N(c4ccc5ccc6ccccc6c5c4)c4ccc(-c5ccccc5)c5sc6cc(-c7ccccc7)ccc6c45)c23)cc1 ; c1ccc(-c2ccc3c(c2)sc2cccc(N(c4ccc(-c5ccc6ccc7ccccc7c6c5)cc4)c4ccc(-c5ccccc5)c5sc6cc(-c7ccccc7)ccc6c45)c23)cc1]

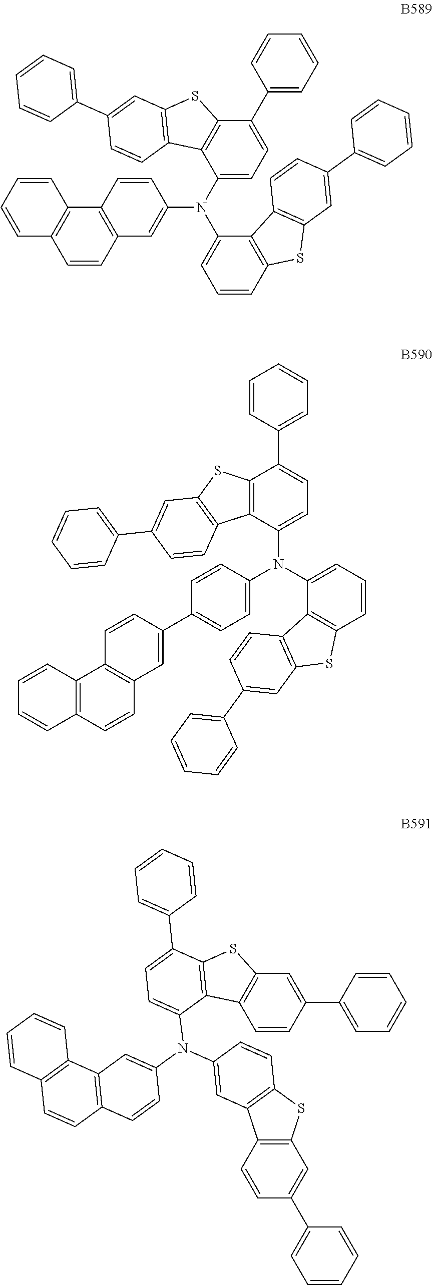 [SMILES: c1ccc(-c2ccc3c(c2)sc2cccc(N(c4ccc5c(ccc6ccccc65)c4)c4ccc(-c5ccccc5)c5sc6cc(-c7ccccc7)ccc6c45)c23)cc1 ; c1ccc(-c2ccc3c(c2)sc2cccc(N(c4ccc(-c5ccc6c(ccc7ccccc76)c5)cc4)c4ccc(-c5ccccc5)c5sc6cc(-c7ccccc7)ccc6c45)c23)cc1 ; c1ccc(-c2ccc3c(c2)sc2ccc(N(c4ccc5ccc6ccccc6c5c4)c4ccc(-c5ccccc5)c5sc6cc(-c7ccccc7)ccc6c45)cc23)cc1]

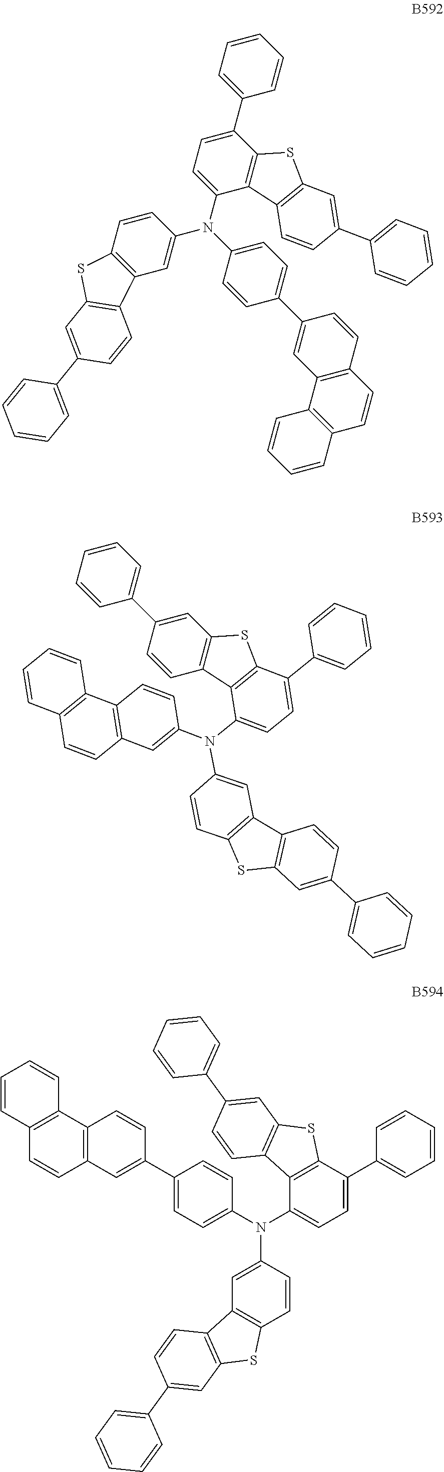 [SMILES: c1ccc(-c2ccc3c(c2)sc2ccc(N(c4ccc(-c5ccc6ccc7ccccc7c6c5)cc4)c4ccc(-c5ccccc5)c5sc6cc(-c7ccccc7)ccc6c45)cc23)cc1 ; c1ccc(-c2ccc3c(c2)sc2ccc(N(c4ccc5c(ccc6ccccc65)c4)c4ccc(-c5ccccc5)c5sc6cc(-c7ccccc7)ccc6c45)cc23)cc1 ; c1ccc(-c2ccc3c(c2)sc2ccc(N(c4ccc(-c5ccc6c(ccc7ccccc76)c5)cc4)c4ccc(-c5ccccc5)c5sc6cc(-c7ccccc7)ccc6c45)cc23)cc1]

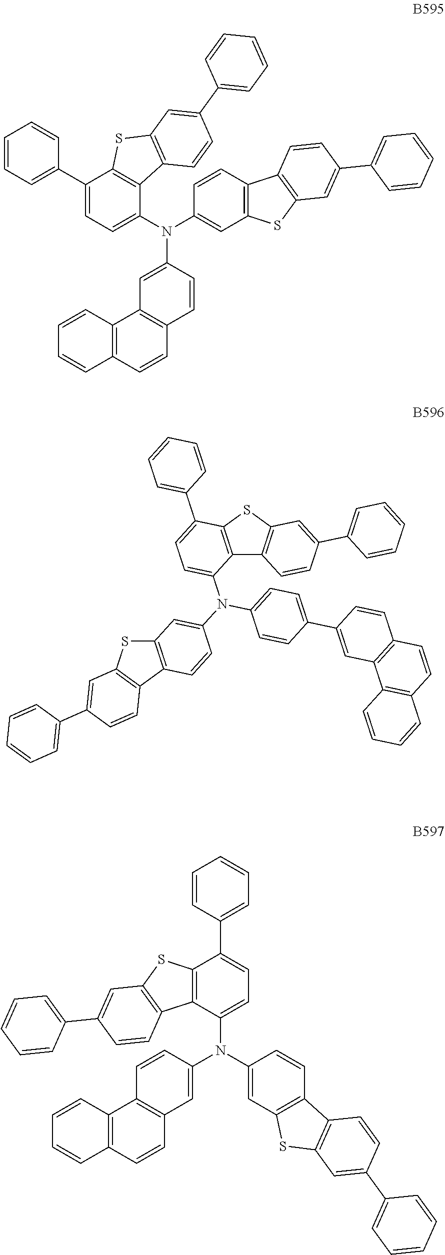 [SMILES: c1ccc(-c2ccc3c(c2)sc2cc(N(c4ccc5ccc6ccccc6c5c4)c4ccc(-c5ccccc5)c5sc6cc(-c7ccccc7)ccc6c45)ccc23)cc1 ; c1ccc(-c2ccc3c(c2)sc2cc(N(c4ccc(-c5ccc6ccc7ccccc7c6c5)cc4)c4ccc(-c5ccccc5)c5sc6cc(-c7ccccc7)ccc6c45)ccc23)cc1 ; c1ccc(-c2ccc3c(c2)sc2cc(N(c4ccc5c(ccc6ccccc65)c4)c4ccc(-c5ccccc5)c5sc6cc(-c7ccccc7)ccc6c45)ccc23)cc1]

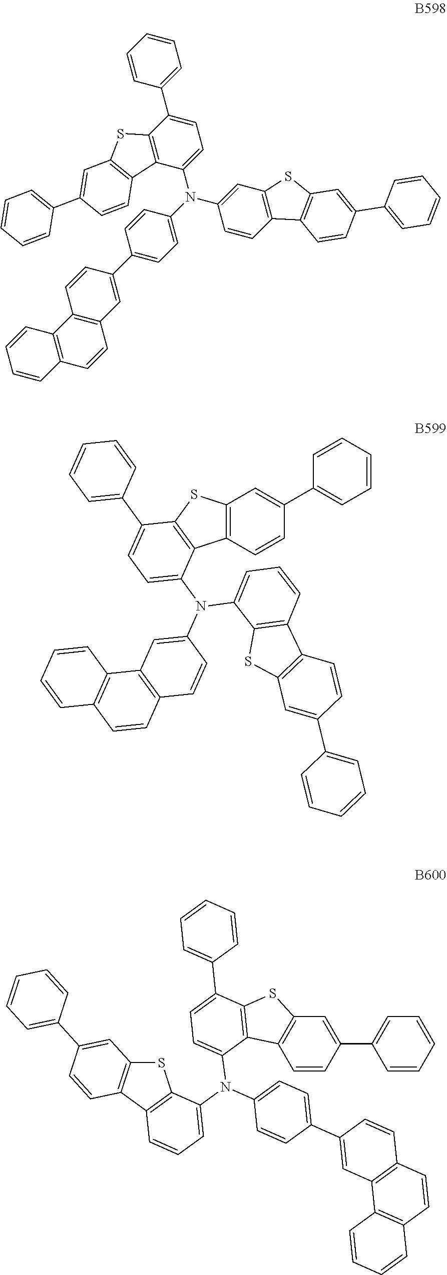 [SMILES: c1ccc(-c2ccc3c(c2)sc2cc(N(c4ccc(-c5ccc6c(ccc7ccccc76)c5)cc4)c4ccc(-c5ccccc5)c5sc6cc(-c7ccccc7)ccc6c45)ccc23)cc1 ; c1ccc(-c2ccc3c(c2)sc2c(N(c4ccc5ccc6ccccc6c5c4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6ccc7ccccc7c6c5)cc4)c4ccc(-c5ccccc5)c5sc6cc(-c7ccccc7)ccc6c45)cccc23)cc1]

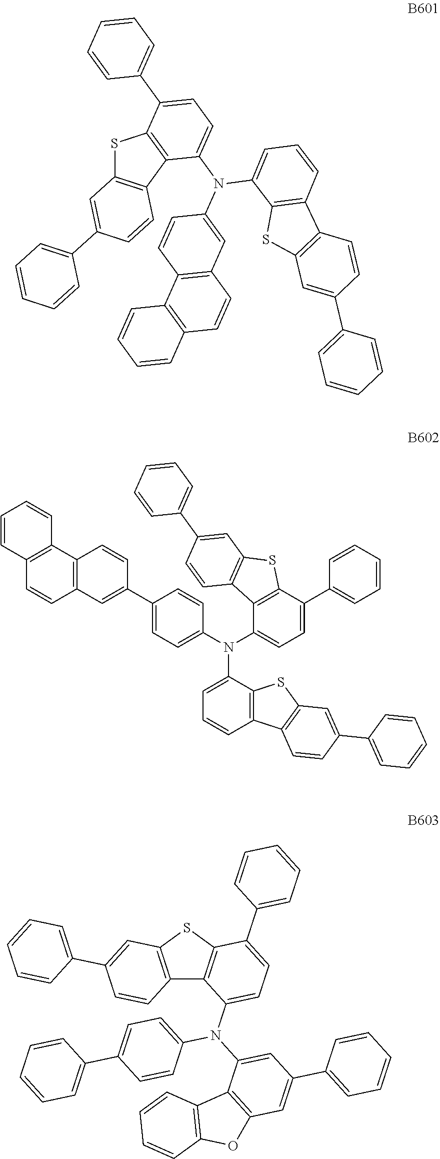 [SMILES: c1ccc(-c2ccc3c(c2)sc2c(N(c4ccc5c(ccc6ccccc65)c4)c4ccc(-c5ccccc5)c5sc6cc(-c7ccccc7)ccc6c45)cccc23)cc1 ; c1ccc(-c2ccc3c(c2)sc2c(N(c4ccc(-c5ccc6c(ccc7ccccc76)c5)cc4)c4ccc(-c5ccccc5)c5sc6cc(-c7ccccc7)ccc6c45)cccc23)cc1 ; c1ccc(-c2ccc(N(c3cc(-c4ccccc4)cc4oc5ccccc5c34)c3ccc(-c4ccccc4)c4sc5cc(-c6ccccc6)ccc5c34)cc2)cc1]

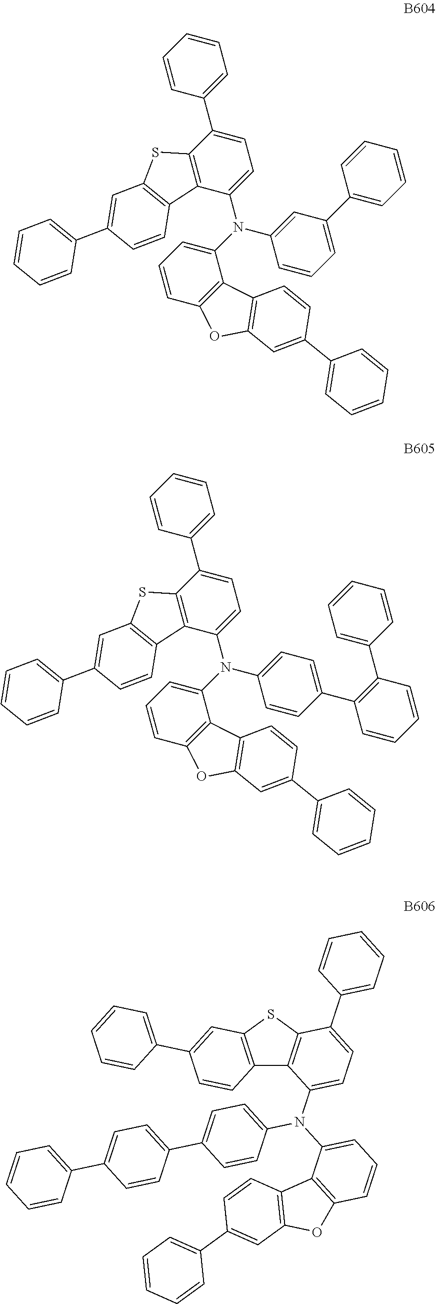 [SMILES: c1ccc(-c2cccc(N(c3cccc4oc5cc(-c6ccccc6)ccc5c34)c3ccc(-c4ccccc4)c4sc5cc(-c6ccccc6)ccc5c34)c2)cc1 ; c1ccc(-c2ccc3c(c2)oc2cccc(N(c4ccc(-c5ccccc5-c5ccccc5)cc4)c4ccc(-c5ccccc5)c5sc6cc(-c7ccccc7)ccc6c45)c23)cc1 ; c1ccc(-c2ccc(-c3ccc(N(c4cccc5oc6cc(-c7ccccc7)ccc6c45)c4ccc(-c5ccccc5)c5sc6cc(-c7ccccc7)ccc6c45)cc3)cc2)cc1]

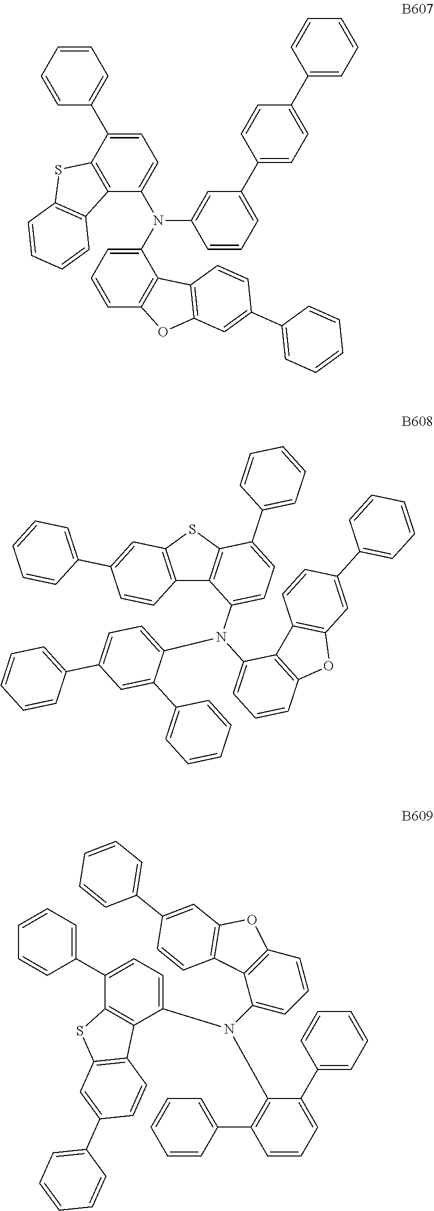 [SMILES: c1ccc(-c2ccc(-c3cccc(N(c4cccc5oc6cc(-c7ccccc7)ccc6c45)c4ccc(-c5ccccc5)c5sc6ccccc6c45)c3)cc2)cc1 ; c1ccc(-c2ccc(N(c3cccc4oc5cc(-c6ccccc6)ccc5c34)c3ccc(-c4ccccc4)c4sc5cc(-c6ccccc6)ccc5c34)c(-c3ccccc3)c2)cc1 ; c1ccc(-c2ccc3c(c2)oc2cccc(N(c4c(-c5ccccc5)cccc4-c4ccccc4)c4ccc(-c5ccccc5)c5sc6cc(-c7ccccc7)ccc6c45)c23)cc1]

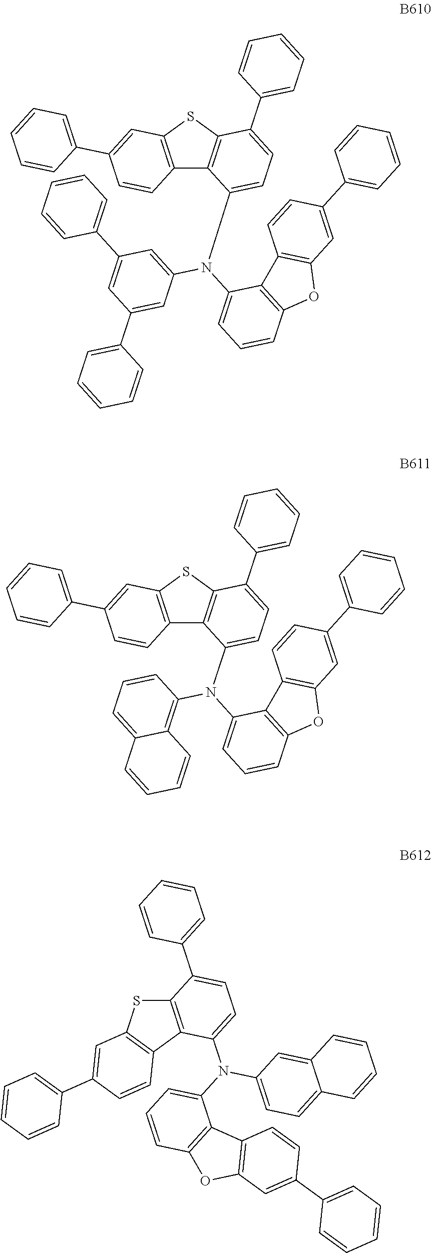 [SMILES: c1ccc(-c2cc(-c3ccccc3)cc(N(c3cccc4oc5cc(-c6ccccc6)ccc5c34)c3ccc(-c4ccccc4)c4sc5cc(-c6ccccc6)ccc5c34)c2)cc1 ; c1ccc(-c2ccc3c(c2)oc2cccc(N(c4cccc5ccccc45)c4ccc(-c5ccccc5)c5sc6cc(-c7ccccc7)ccc6c45)c23)cc1 ; c1ccc(-c2ccc3c(c2)oc2cccc(N(c4ccc5ccccc5c4)c4ccc(-c5ccccc5)c5sc6cc(-c7ccccc7)ccc6c45)c23)cc1]

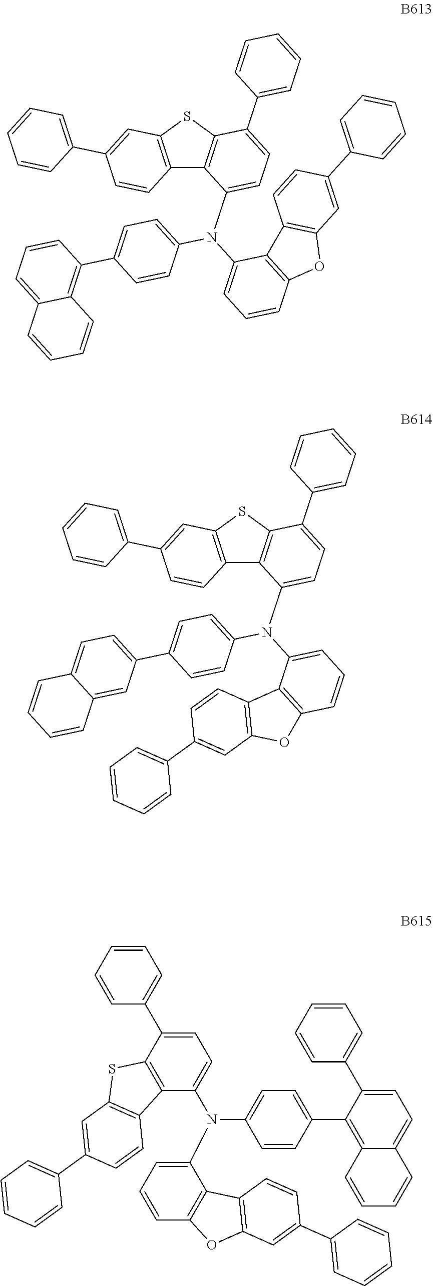 [SMILES: c1ccc(-c2ccc3c(c2)oc2cccc(N(c4ccc(-c5cccc6ccccc56)cc4)c4ccc(-c5ccccc5)c5sc6cc(-c7ccccc7)ccc6c45)c23)cc1 ; c1ccc(-c2ccc3c(c2)oc2cccc(N(c4ccc(-c5ccc6ccccc6c5)cc4)c4ccc(-c5ccccc5)c5sc6cc(-c7ccccc7)ccc6c45)c23)cc1 ; c1ccc(-c2ccc3c(c2)oc2cccc(N(c4ccc(-c5c(-c6ccccc6)ccc6ccccc56)cc4)c4ccc(-c5ccccc5)c5sc6cc(-c7ccccc7)ccc6c45)c23)cc1]

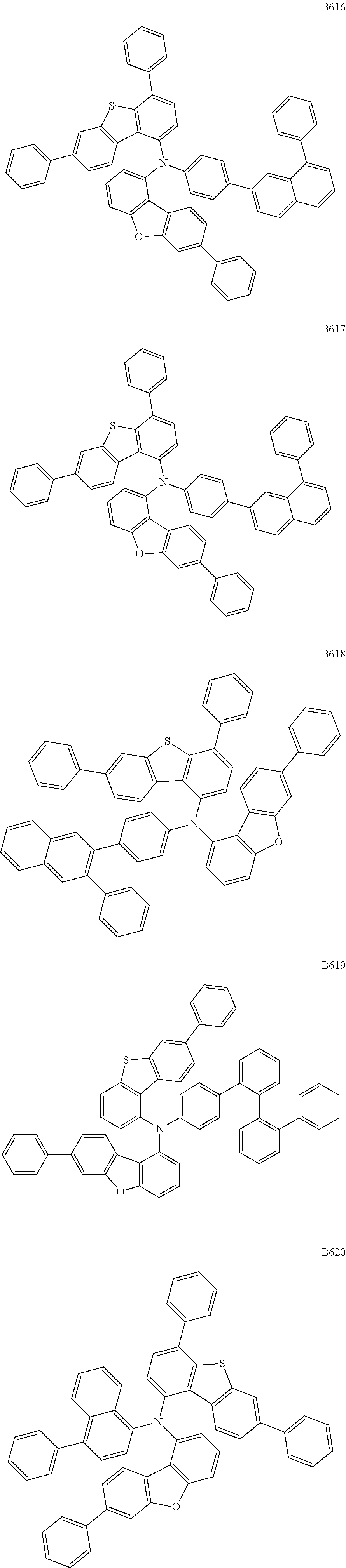

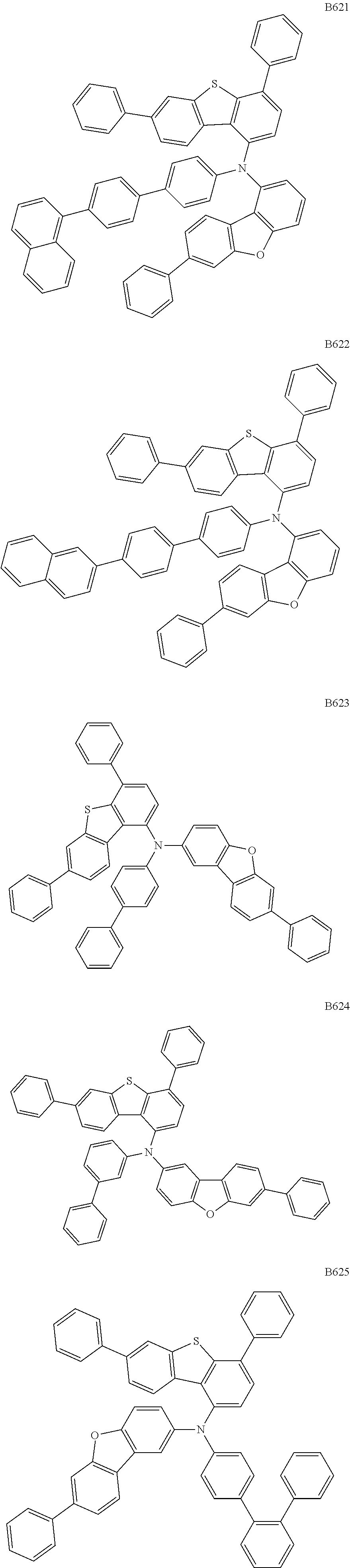

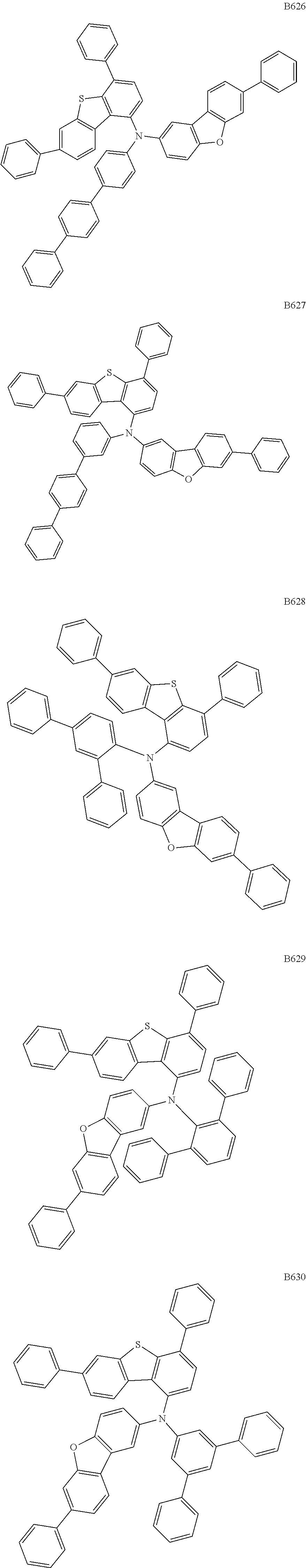

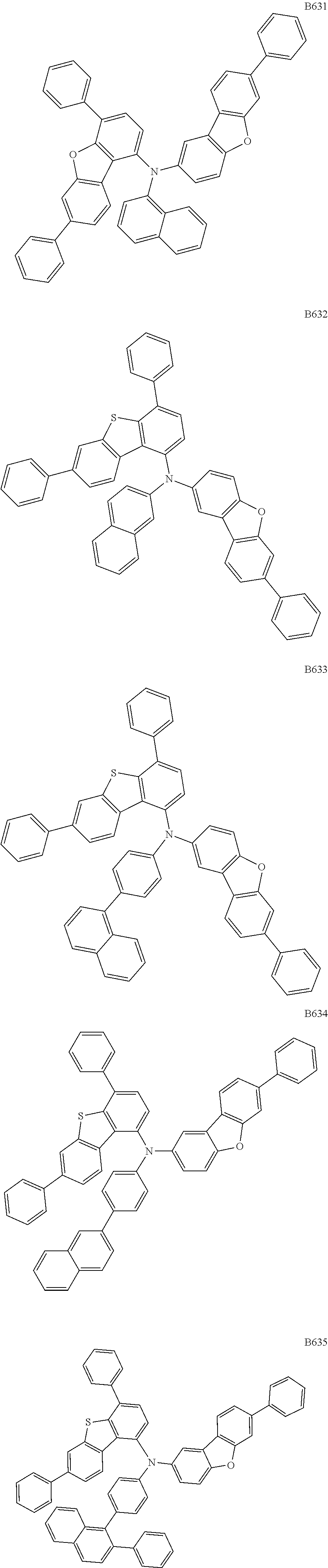

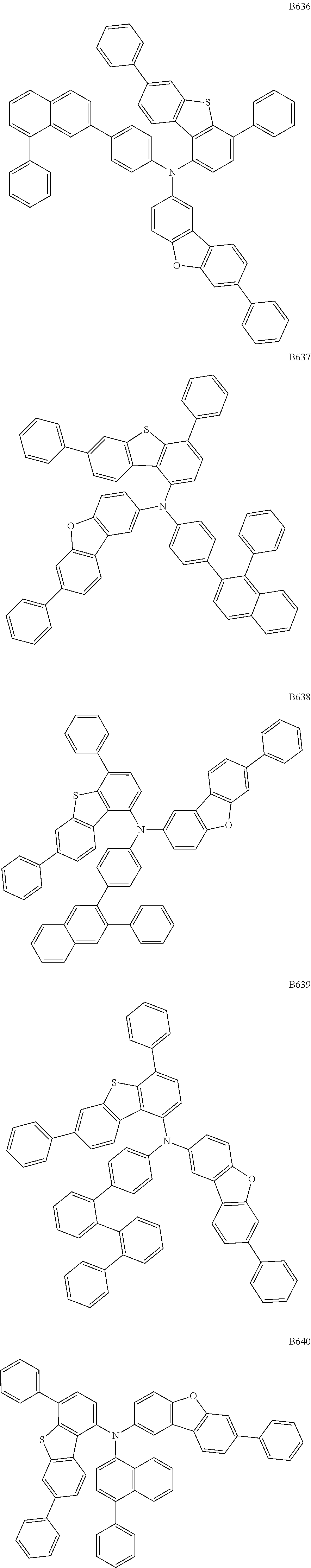

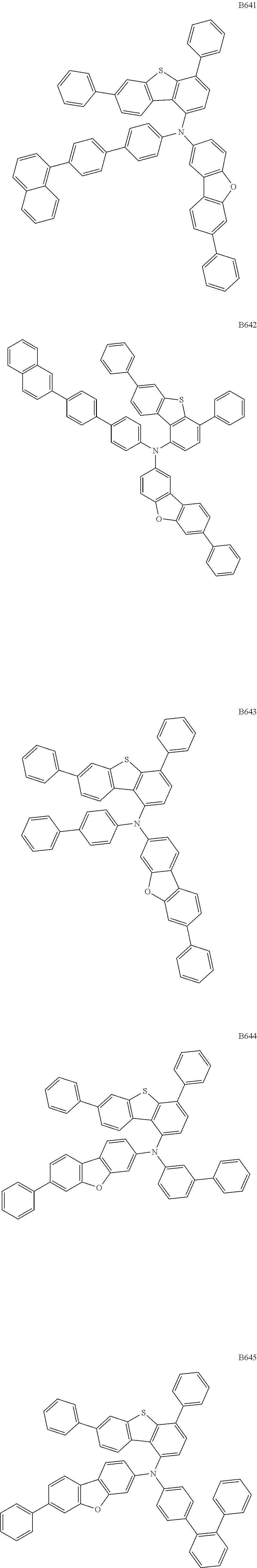

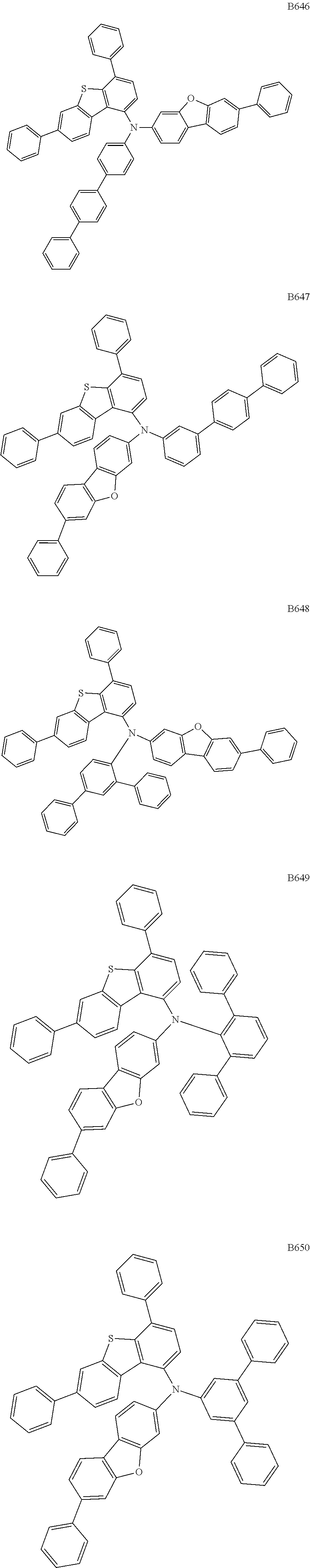

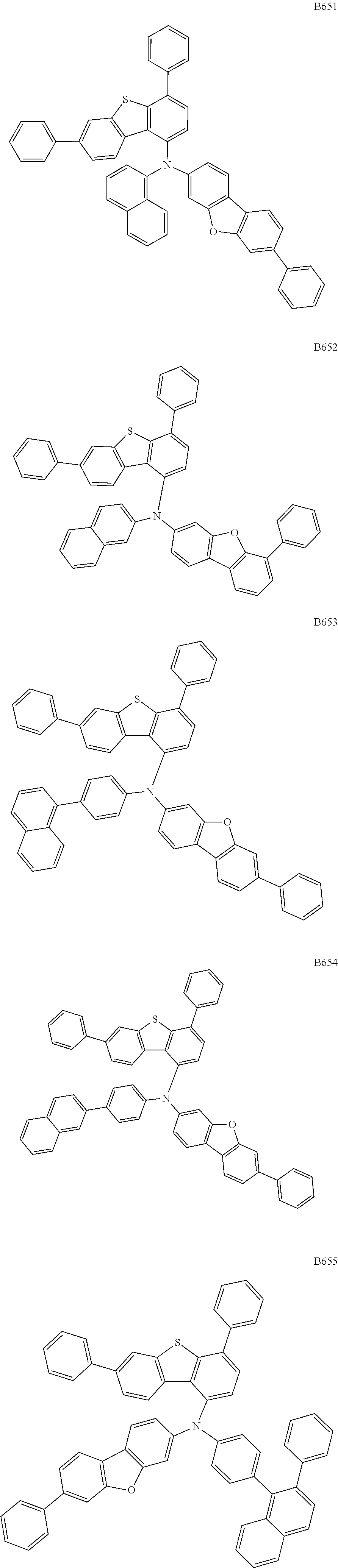

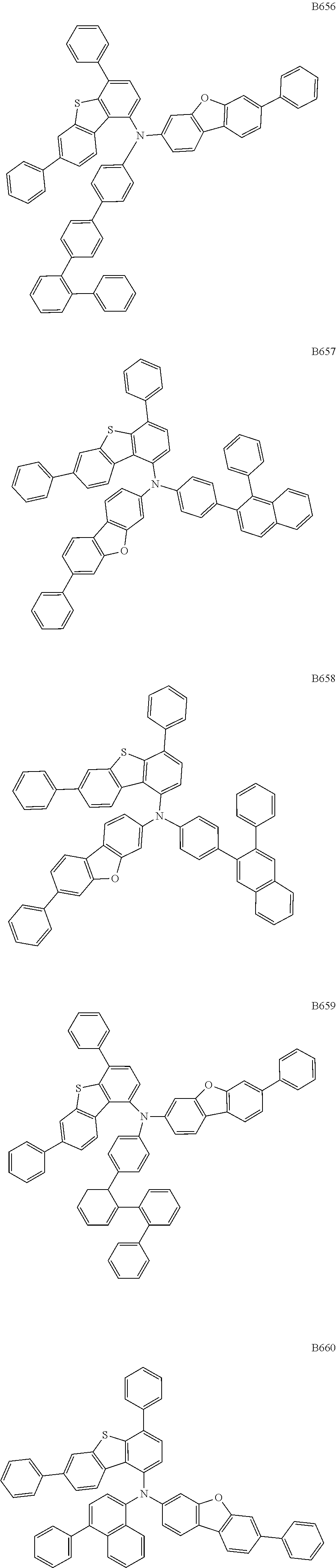

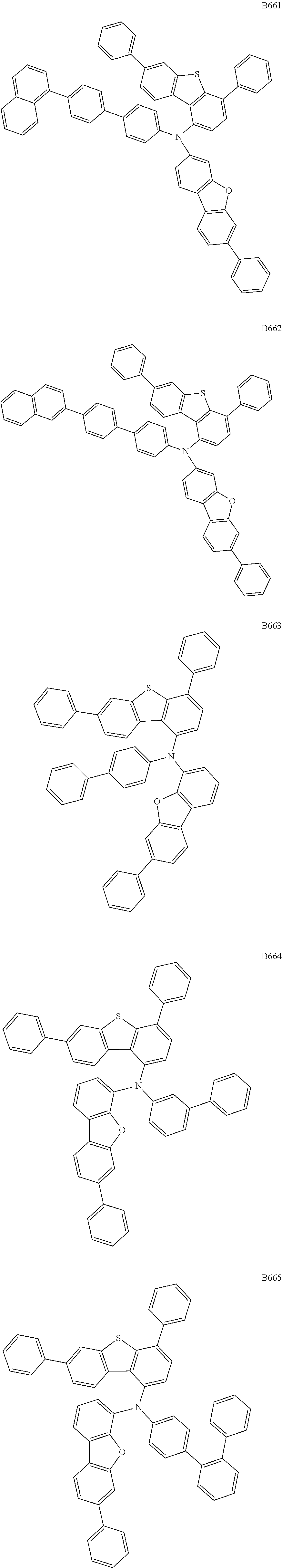

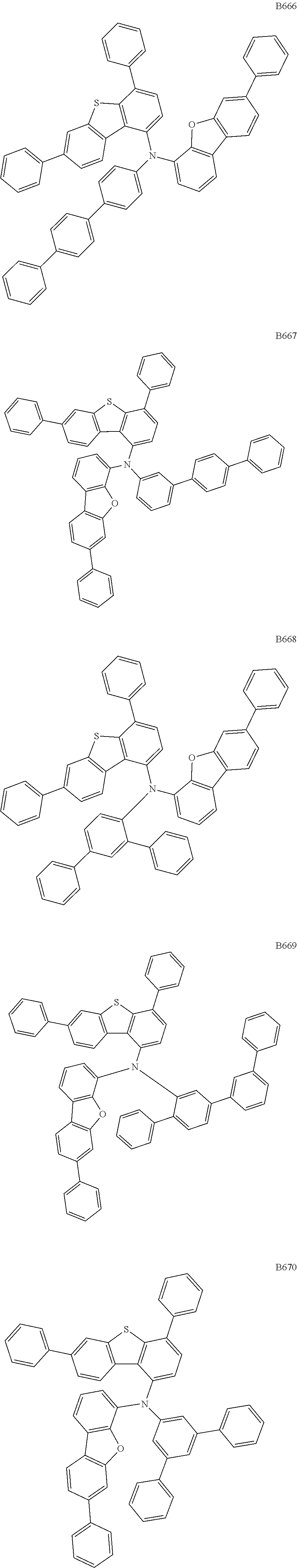

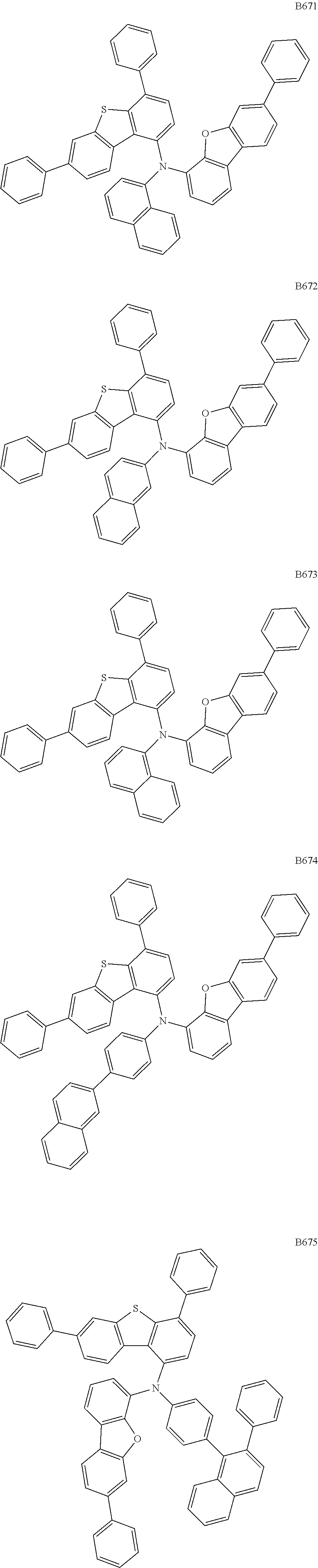

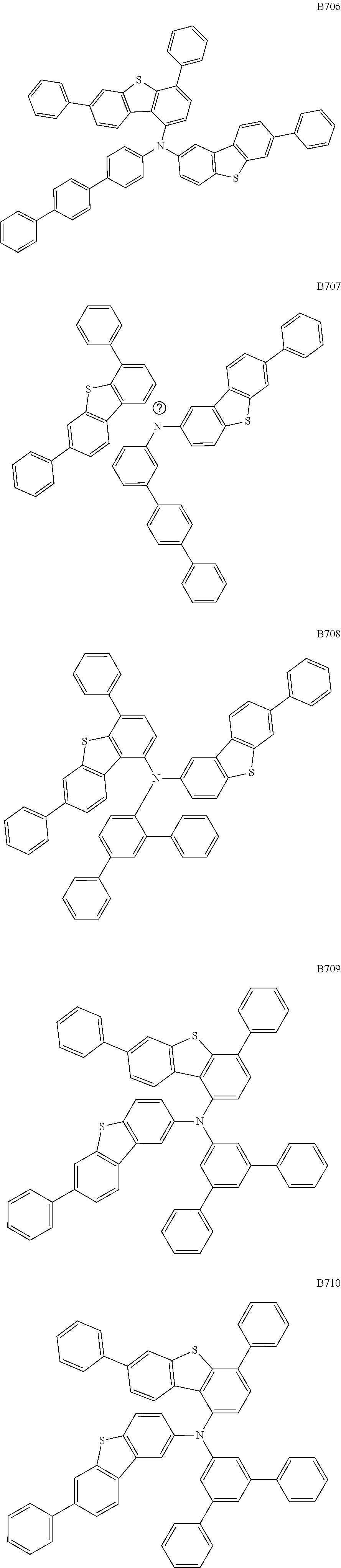

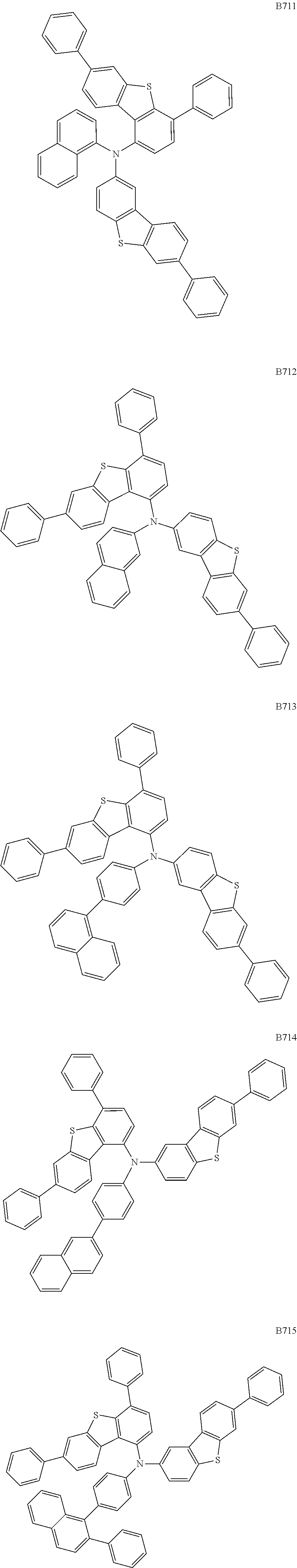

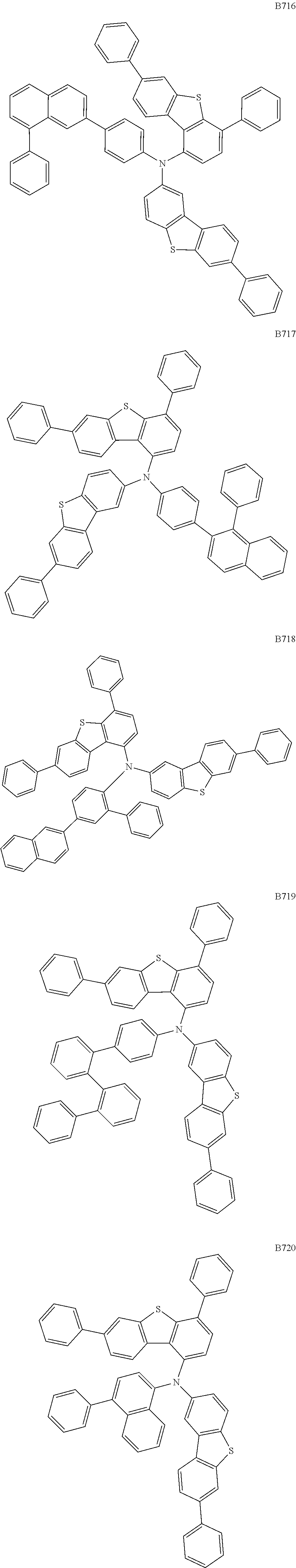

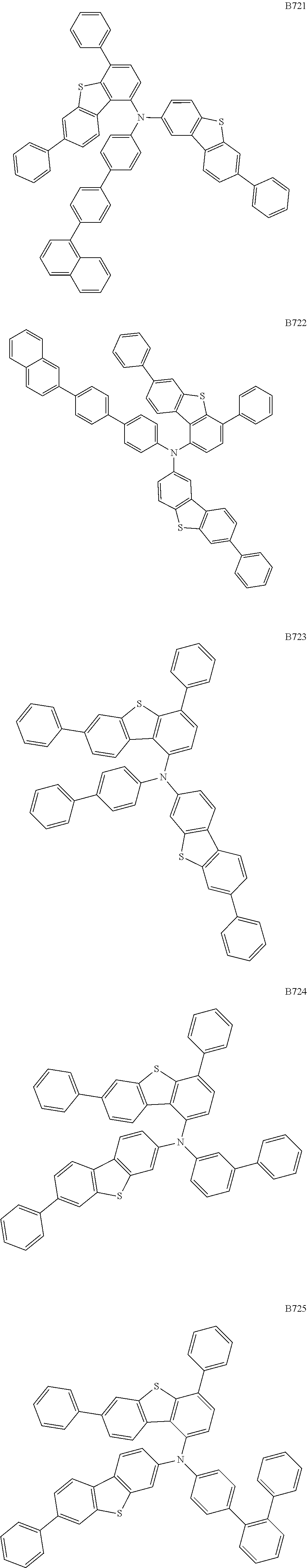

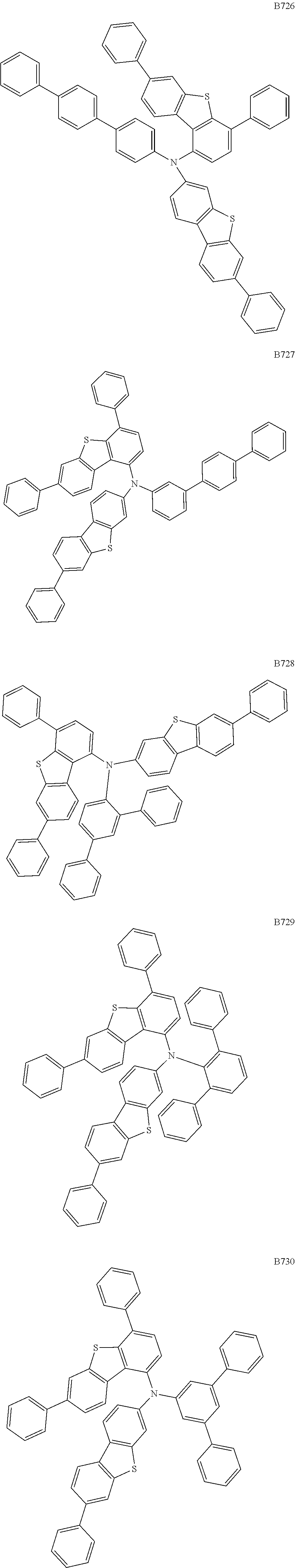

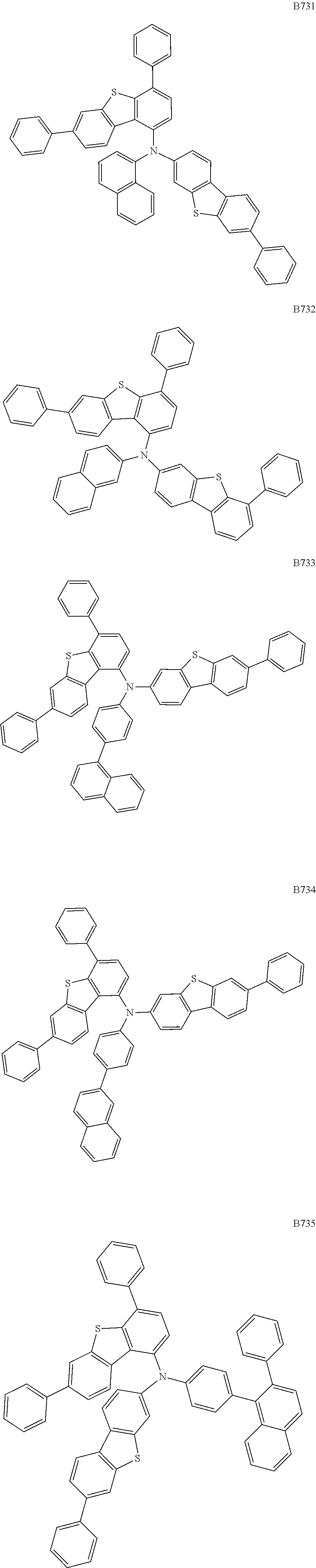

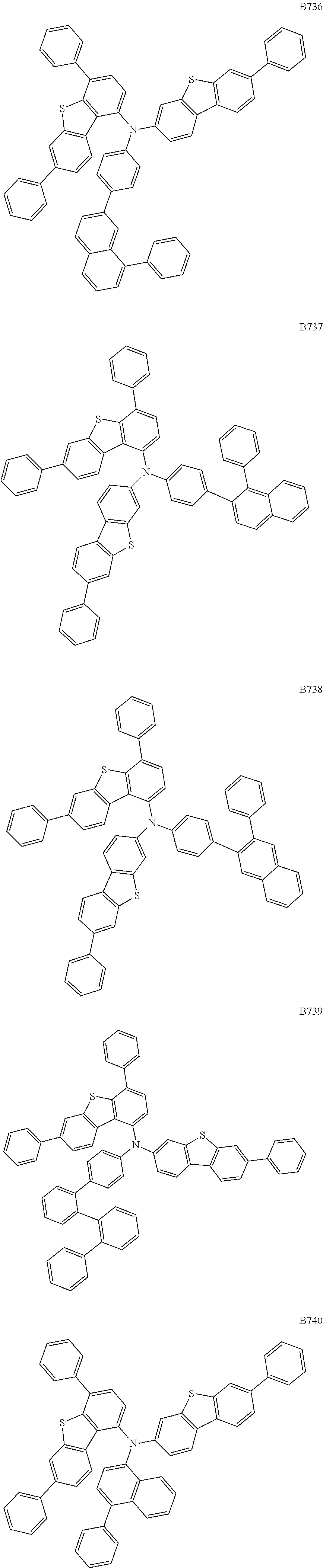

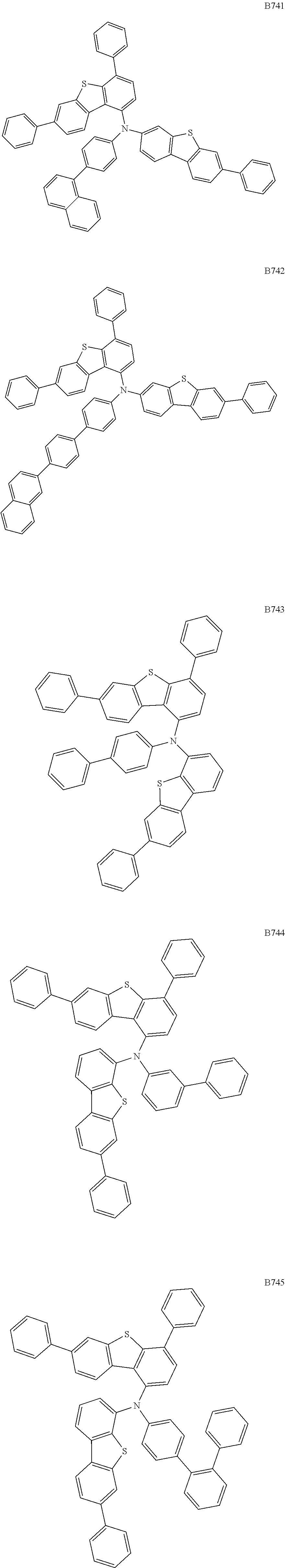

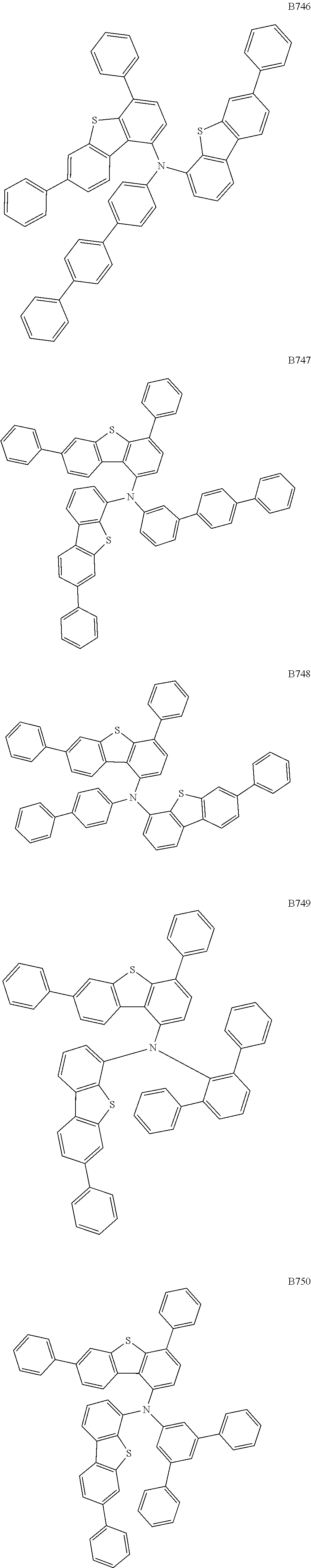

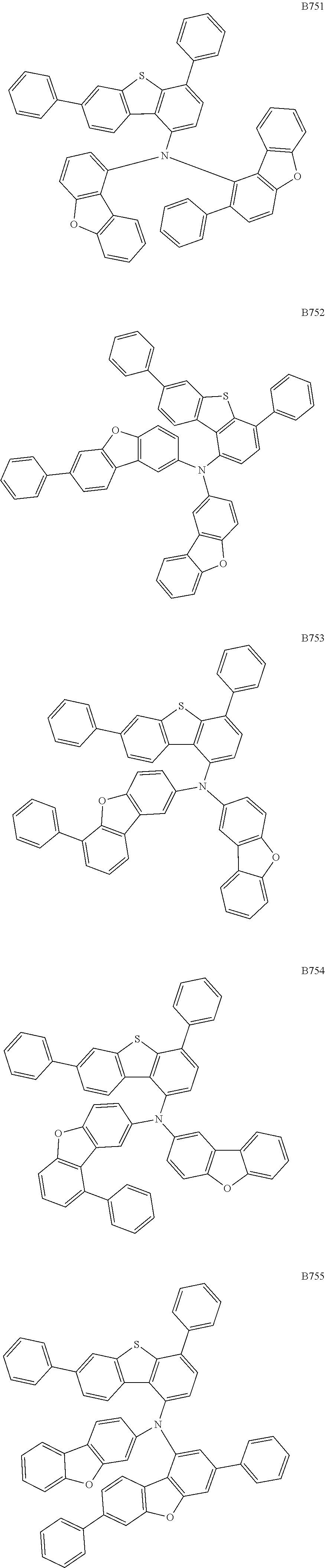

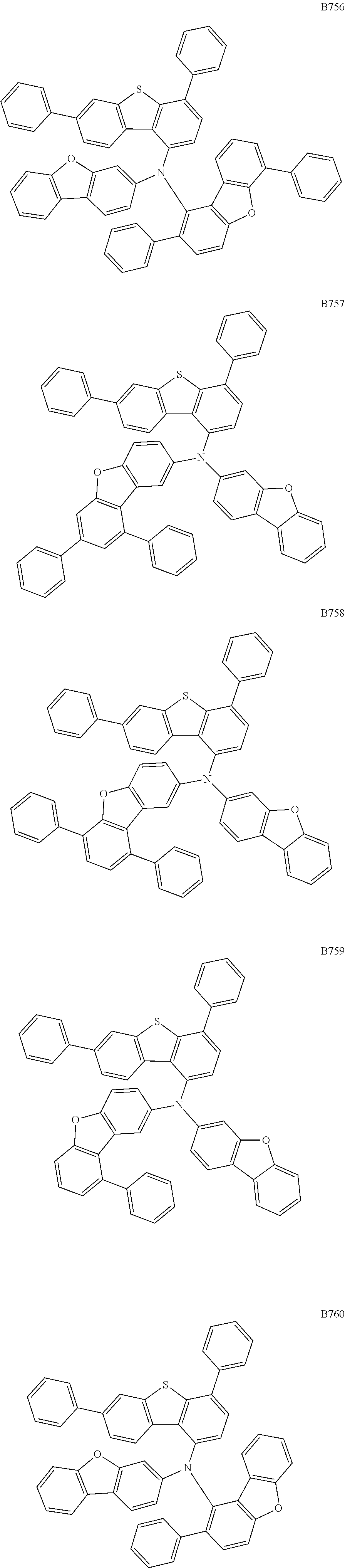

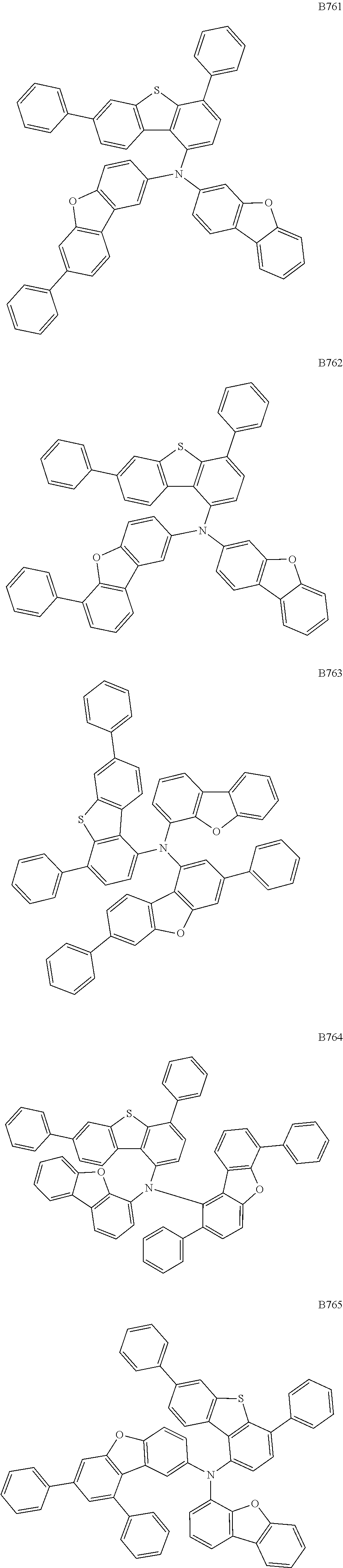

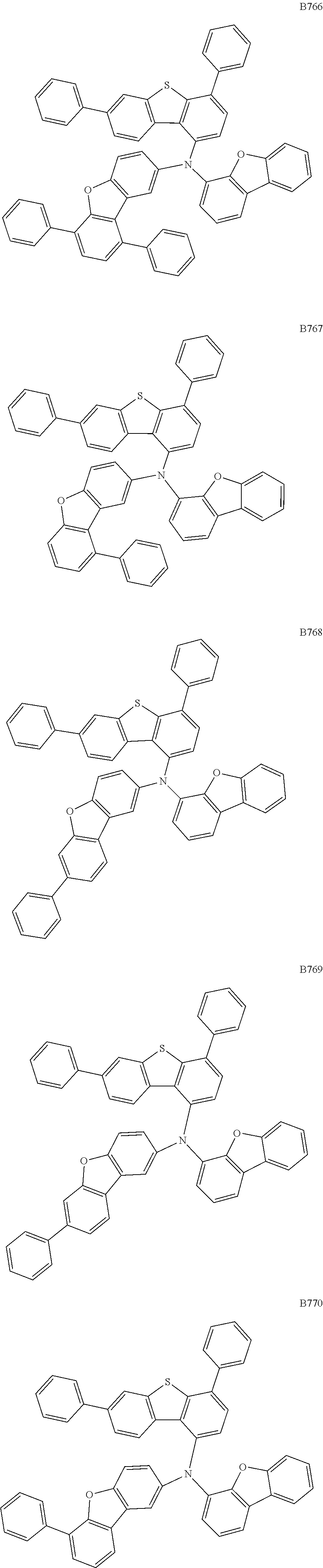

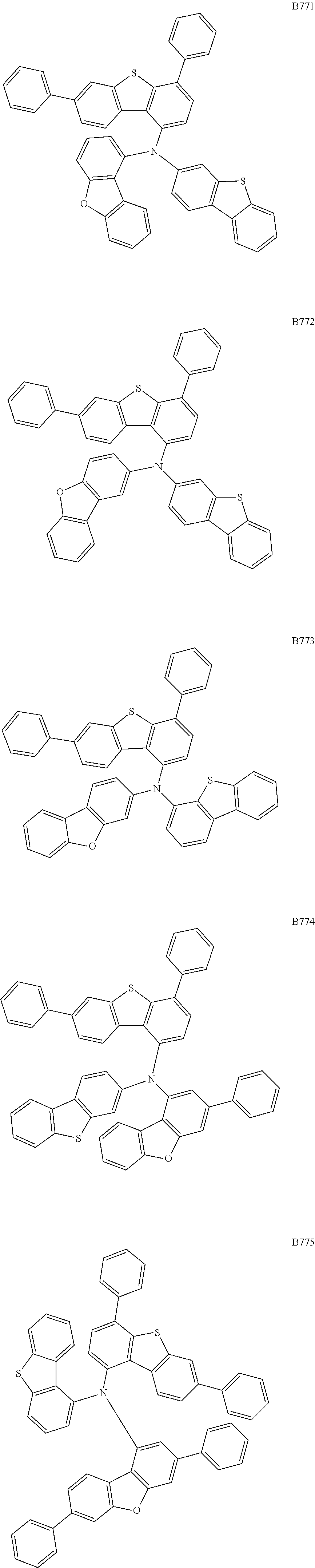 [SMILES: c1ccc(-c2ccc3c(c2)sc2c(-c4ccccc4)ccc(N(c4ccc5c(c4)sc4ccccc45)c4cccc5oc6ccccc6c45)c23)cc1 ; c1ccc(-c2ccc3c(c2)sc2c(-c4ccccc4)ccc(N(c4ccc5c(c4)sc4ccccc45)c4ccc5oc6ccccc6c5c4)c23)cc1 ; c1ccc(-c2ccc3c(c2)sc2c(-c4ccccc4)ccc(N(c4ccc5c(c4)oc4ccccc45)c4cccc5c4sc4ccccc45)c23)cc1 ; c1ccc(-c2cc(N(c3ccc4c(c3)sc3ccccc34)c3ccc(-c4ccccc4)c4sc5cc(-c6ccccc6)ccc5c34)c3c(c2)oc2ccccc23)cc1 ; c1ccc(-c2ccc3c(c2)oc2cc(-c4ccccc4)cc(N(c4cccc5sc6ccccc6c45)c4ccc(-c5ccccc5)c5sc6cc(-c7ccccc7)ccc6c45)c23)cc1]

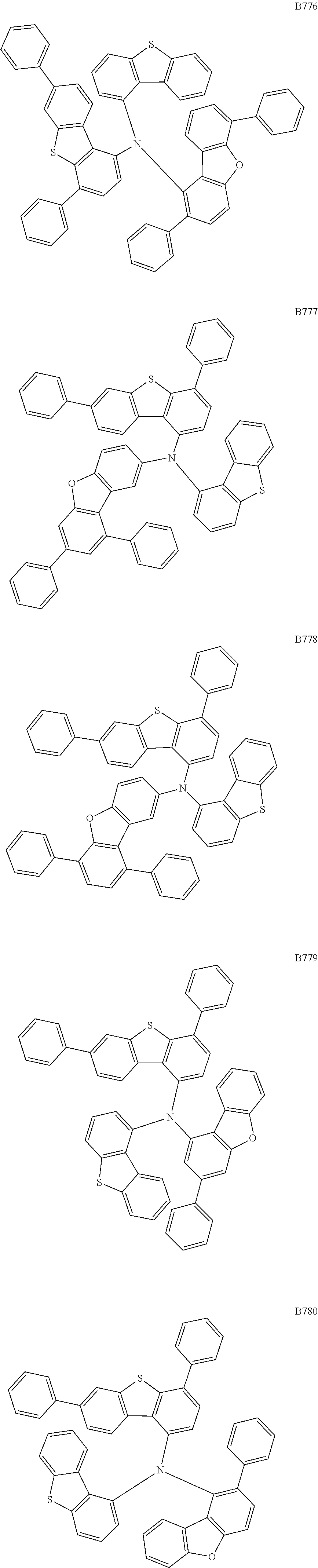

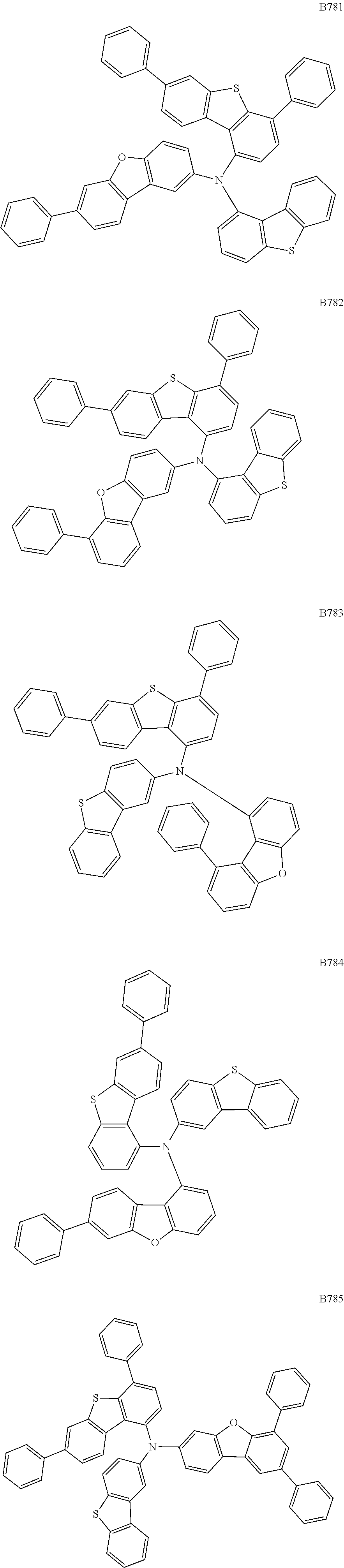 [SMILES: c1ccc(-c2ccc3c(c2)oc2ccc(N(c4cccc5sc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5sc6ccccc6c45)c23)cc1 ; c1ccc(-c2ccc3c(c2)sc2c(-c4ccccc4)ccc(N(c4ccc5sc6ccccc6c5c4)c4cccc5oc6cccc(-c7ccccc7)c6c45)c23)cc1 ; c1ccc(-c2ccc3c(c2)oc2cccc(N(c4ccc5sc6ccccc6c5c4)c4cccc5sc6cc(-c7ccccc7)ccc6c45)c23)cc1 ; c1ccc(-c2ccc3c(c2)sc2c(-c4ccccc4)ccc(N(c4ccc5c(c4)oc4c(-c6ccccc6)cc(-c6ccccc6)cc45)c4ccc5sc6ccccc6c5c4)c23)cc1]

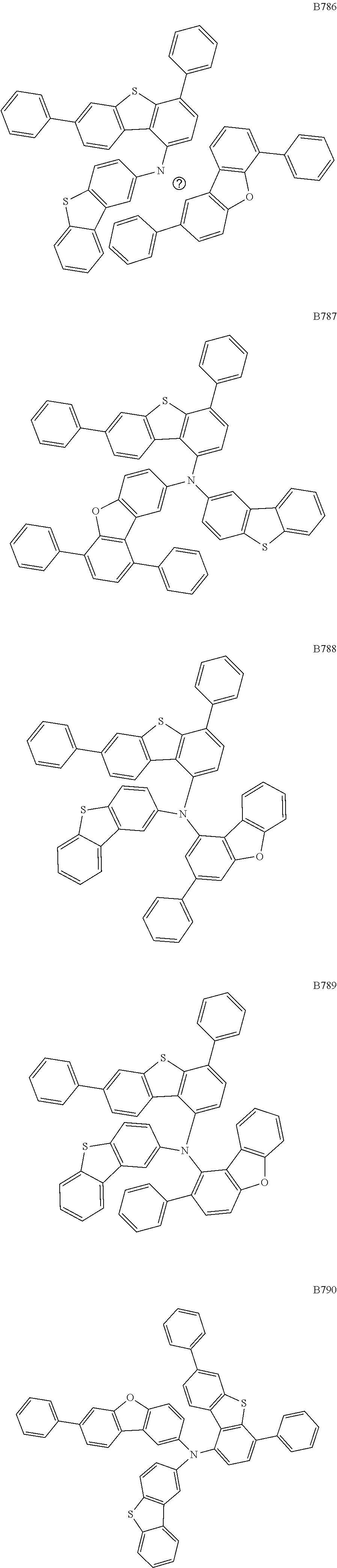

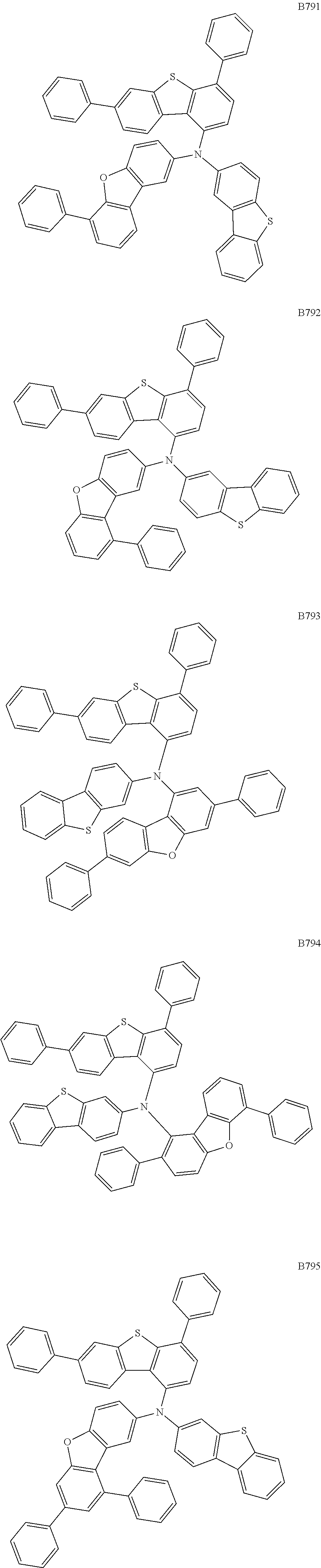

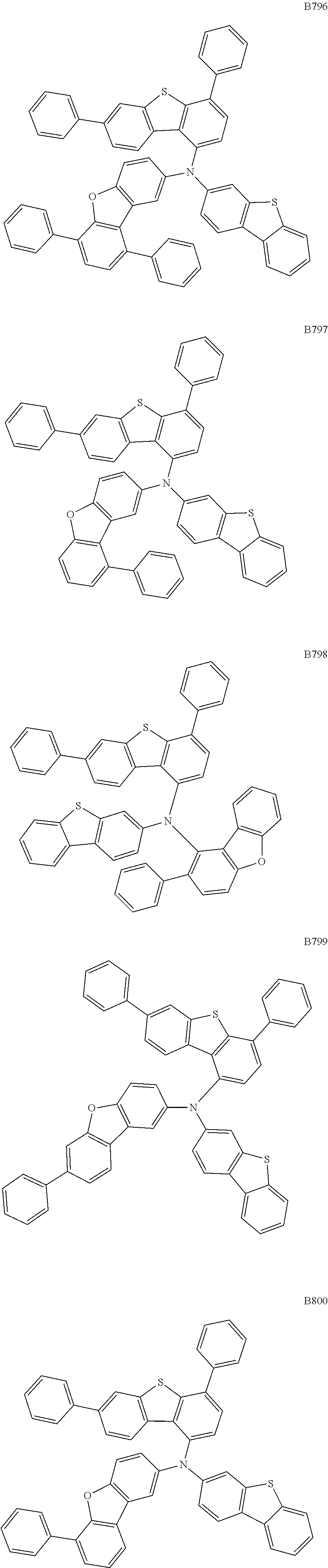

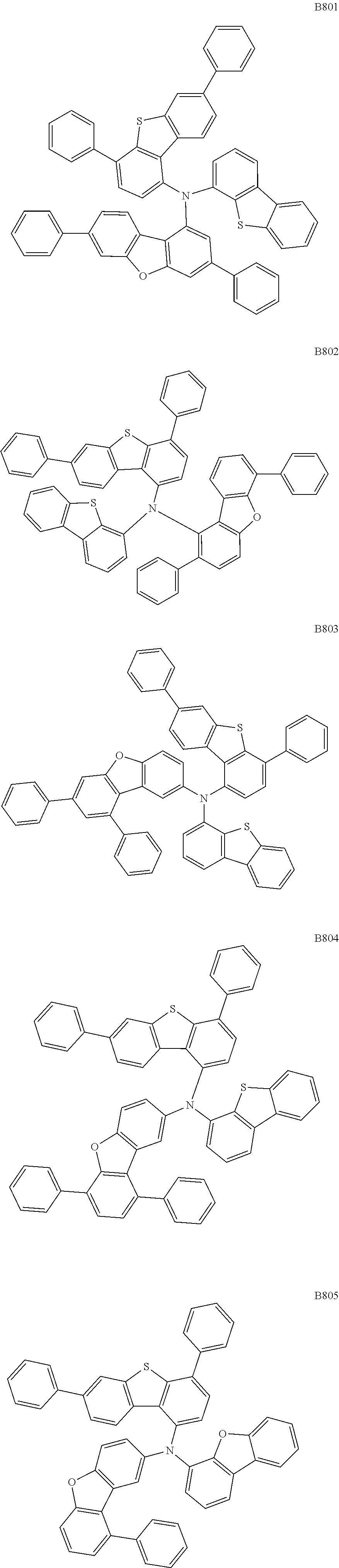

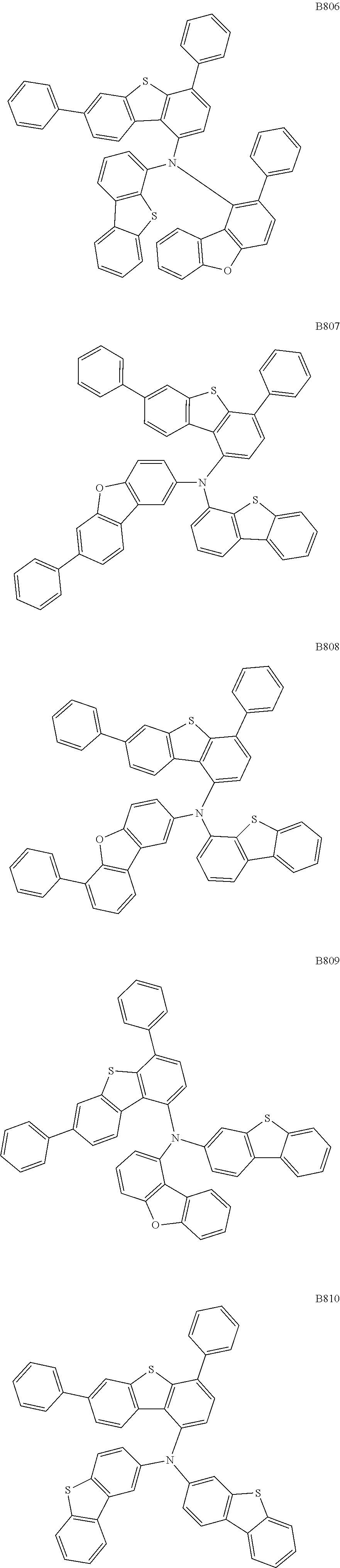 [SMILES: c1ccc(-c2ccc3c(c2)sc2c(-c4ccccc4)ccc(N(c4cccc5c4sc4ccccc45)c4c(-c5ccccc5)ccc5oc6ccccc6c45)c23)cc1 ; c1ccc(-c2ccc3c(c2)oc2ccc(N(c4cccc5c4sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5c4sc4ccccc45)c23)cc1 ; c1ccc(-c2ccc3c(c2)sc2c(-c4ccccc4)ccc(N(c4ccc5c(c4)sc4ccccc45)c4cccc5oc6ccccc6c45)c23)cc1 ; c1ccc(-c2ccc3c(c2)sc2c(-c4ccccc4)ccc(N(c4ccc5c(c4)sc4ccccc45)c4ccc5sc6ccccc6c5c4)c23)cc1]

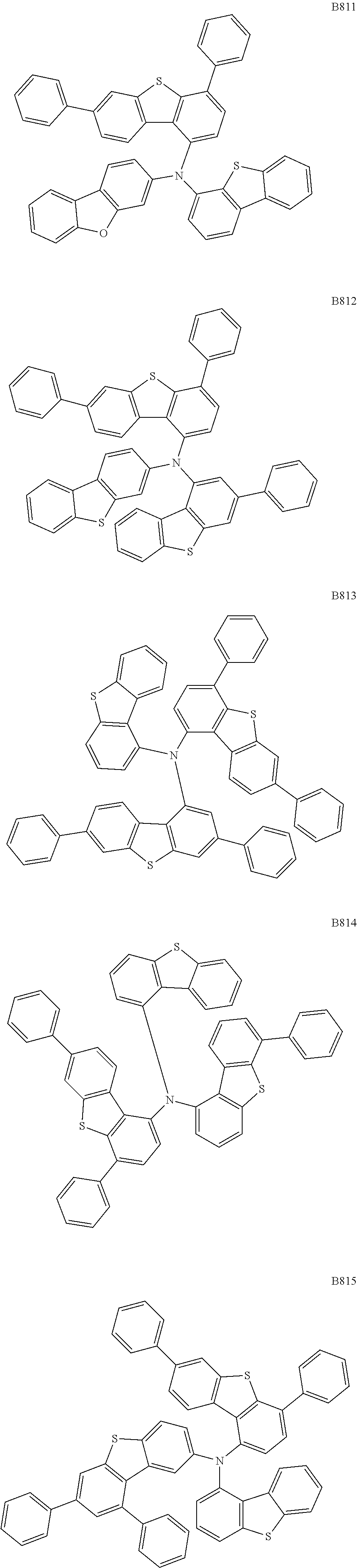

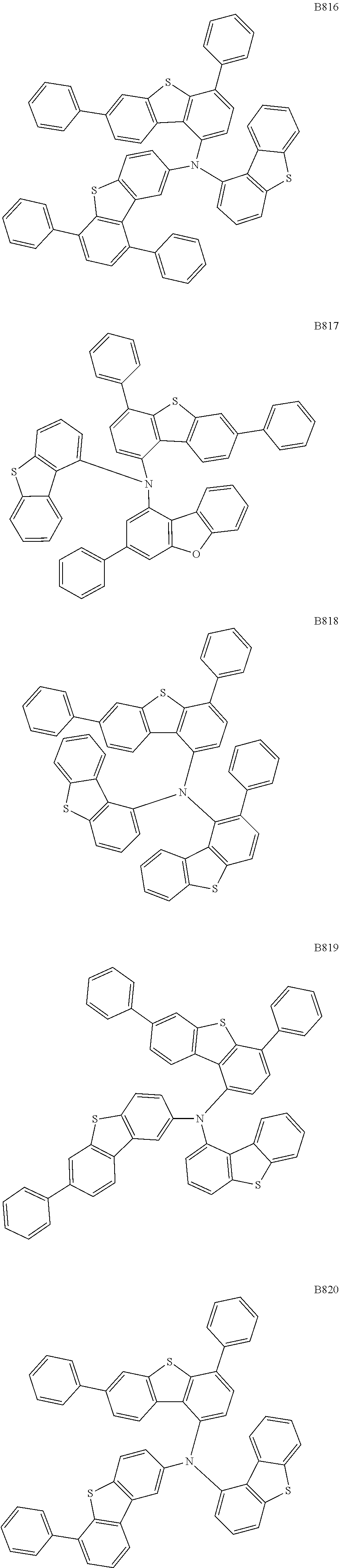

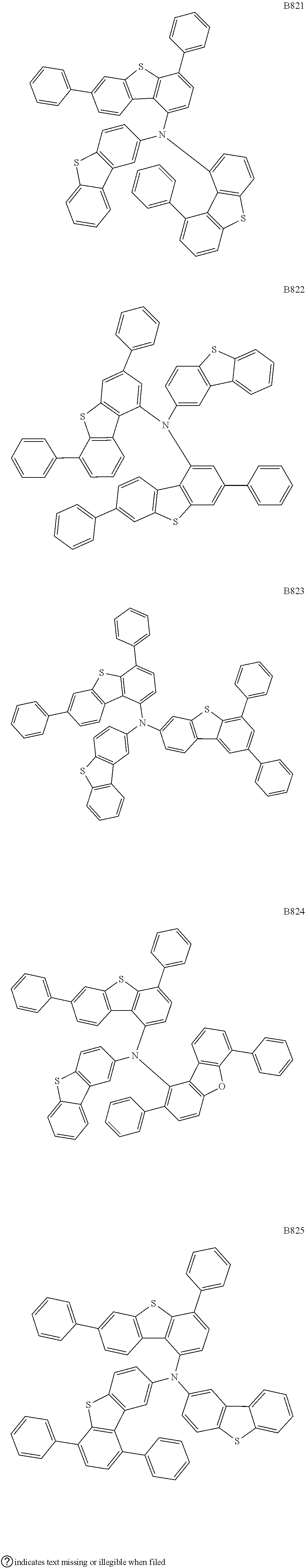

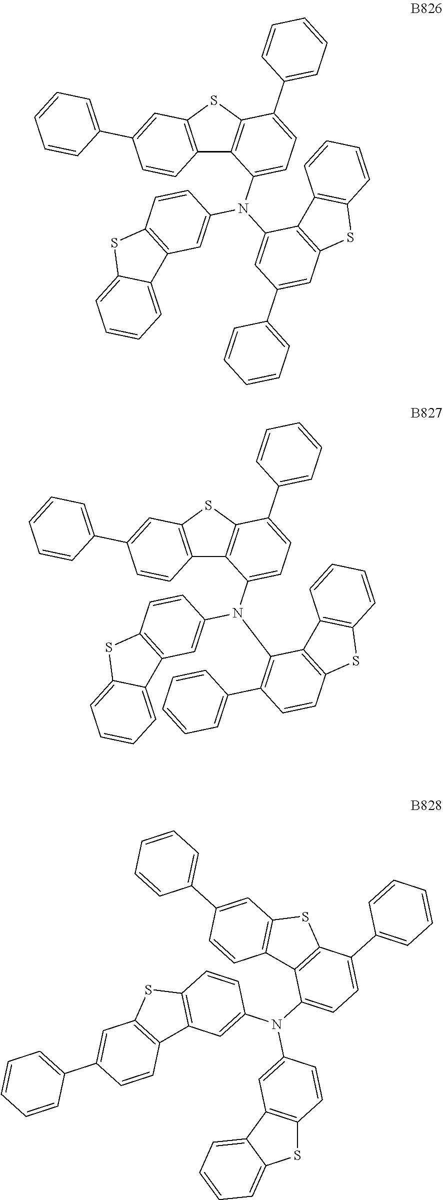 [SMILES: c1ccc(-c2ccc3c(c2)sc2c(-c4ccccc4)ccc(N(c4ccc5sc6ccccc6c5c4)c4cc(-c5ccccc5)cc5sc6ccccc6c45)c23)cc1 ; c1ccc(-c2ccc3c(c2)sc2c(-c4ccccc4)ccc(N(c4ccc5sc6ccccc6c5c4)c4c(-c5ccccc5)ccc5sc6ccccc6c45)c23)cc1 ; c1ccc(-c2ccc3c(c2)sc2ccc(N(c4ccc5sc6ccccc6c5c4)c4ccc(-c5ccccc5)c5sc6cc(-c7ccccc7)ccc6c45)cc23)cc1]

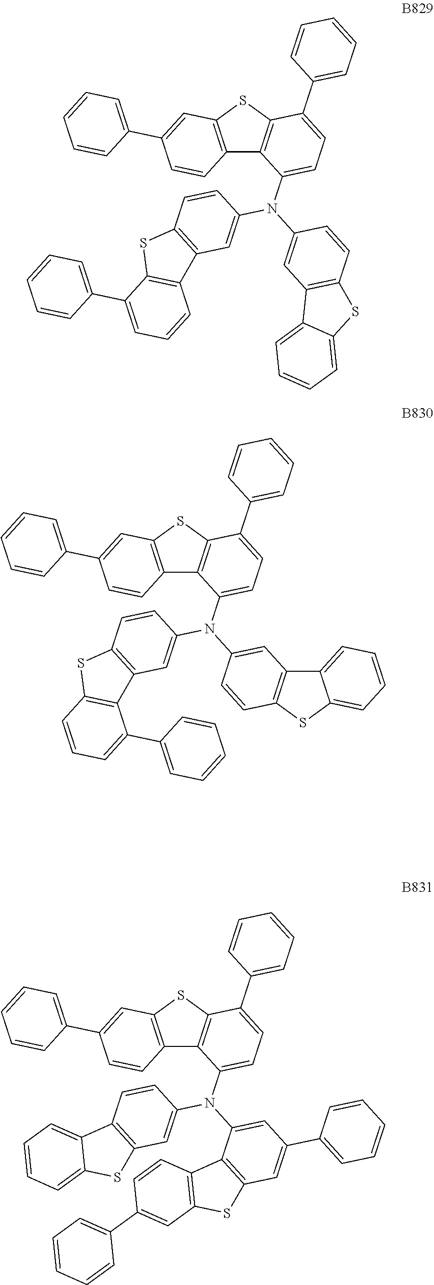 [SMILES: c1ccc(-c2ccc3c(c2)sc2c(-c4ccccc4)ccc(N(c4ccc5sc6ccccc6c5c4)c4ccc5sc6c(-c7ccccc7)cccc6c5c4)c23)cc1 ; c1ccc(-c2ccc3c(c2)sc2c(-c4ccccc4)ccc(N(c4ccc5sc6ccccc6c5c4)c4ccc5sc6cccc(-c7ccccc7)c6c5c4)c23)cc1 ; c1ccc(-c2ccc3c(c2)sc2cc(-c4ccccc4)cc(N(c4ccc5c(c4)sc4ccccc45)c4ccc(-c5ccccc5)c5sc6cc(-c7ccccc7)ccc6c45)c23)cc1]

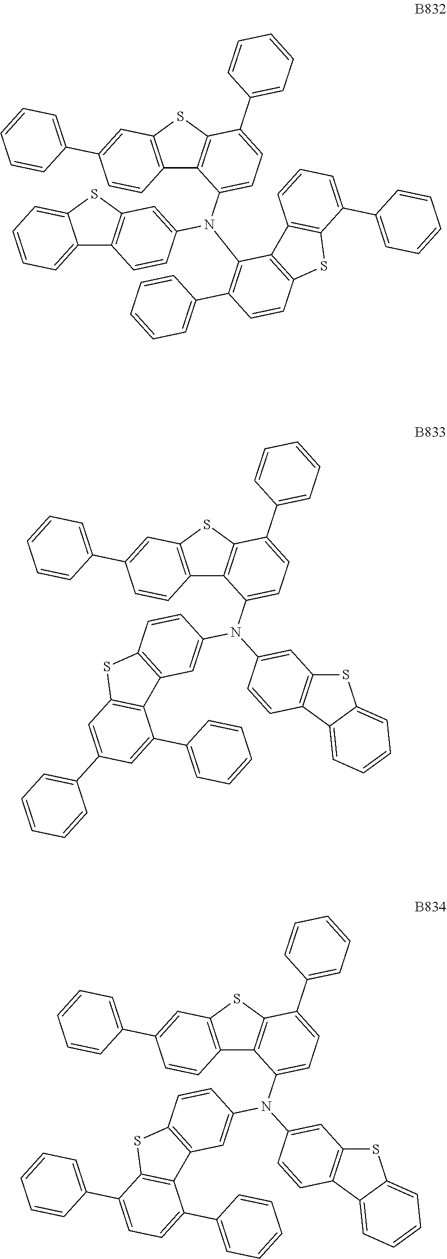 [SMILES: c1ccc(-c2ccc3c(c2)sc2c(-c4ccccc4)ccc(N(c4ccc5c(c4)sc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5c(c4)sc4ccccc45)c4ccc5sc6cc(-c7ccccc7)cc(-c7ccccc7)c6c5c4)c23)cc1 ; c1ccc(-c2ccc3c(c2)sc2c(-c4ccccc4)ccc(N(c4ccc5c(c4)sc4ccccc45)c4ccc5sc6c(-c7ccccc7)ccc(-c7ccccc7)c6c5c4)c23)cc1]

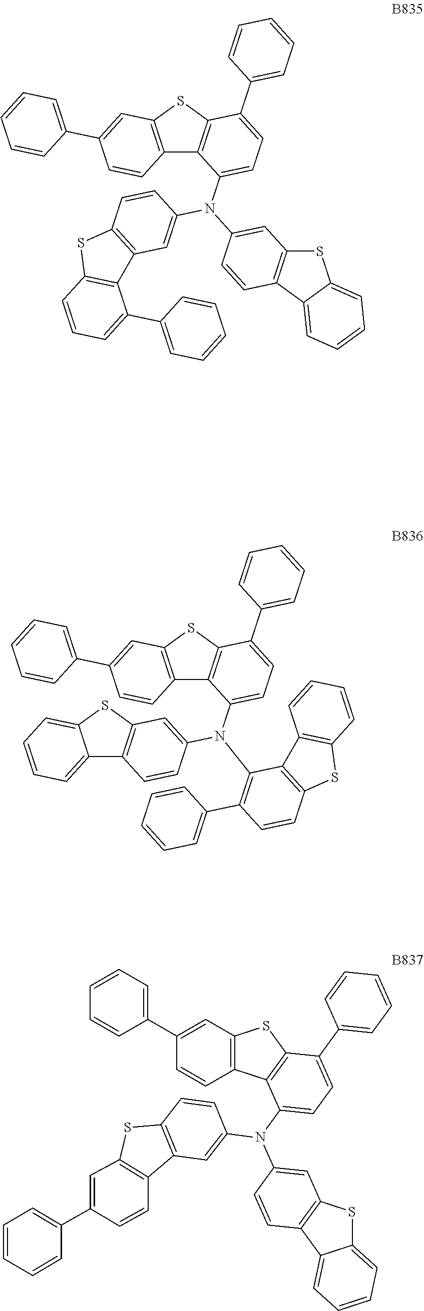 [SMILES: c1ccc(-c2ccc3c(c2)sc2c(-c4ccccc4)ccc(N(c4ccc5c(c4)sc4ccccc45)c4ccc5sc6cccc(-c7ccccc7)c6c5c4)c23)cc1 ; c1ccc(-c2ccc3c(c2)sc2c(-c4ccccc4)ccc(N(c4ccc5c(c4)sc4ccccc45)c4c(-c5ccccc5)ccc5sc6ccccc6c45)c23)cc1 ; c1ccc(-c2ccc3c(c2)sc2ccc(N(c4ccc5c(c4)sc4ccccc45)c4ccc(-c5ccccc5)c5sc6cc(-c7ccccc7)ccc6c45)cc23)cc1]

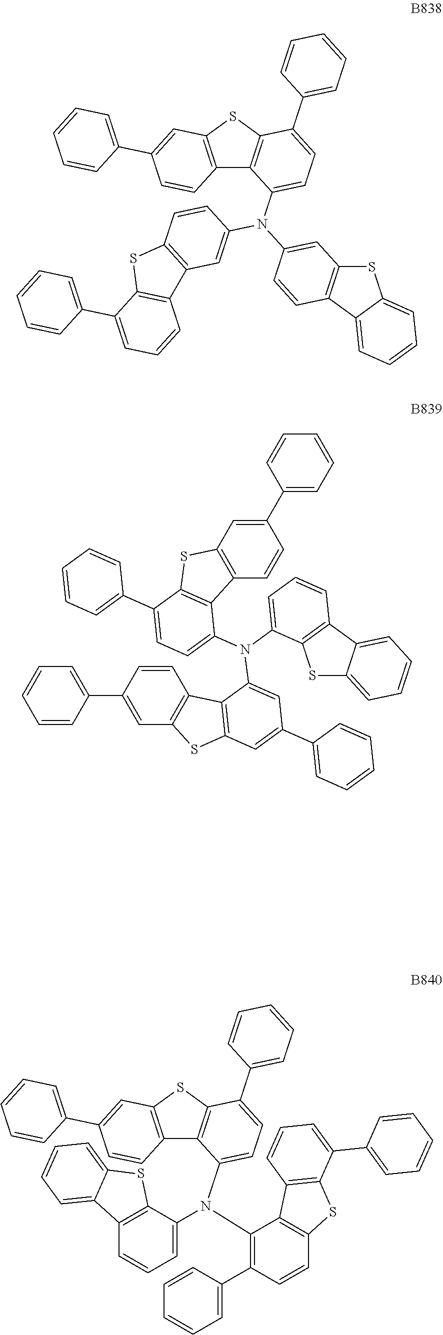 [SMILES: c1ccc(-c2ccc3c(c2)sc2c(-c4ccccc4)ccc(N(c4ccc5c(c4)sc4ccccc45)c4ccc5sc6c(-c7ccccc7)cccc6c5c4)c23)cc1 ; c1ccc(-c2ccc3c(c2)sc2cc(-c4ccccc4)cc(N(c4cccc5c4sc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5c4sc4ccccc45)c4c(-c5ccccc5)ccc5sc6c(-c7ccccc7)cccc6c45)c23)cc1]

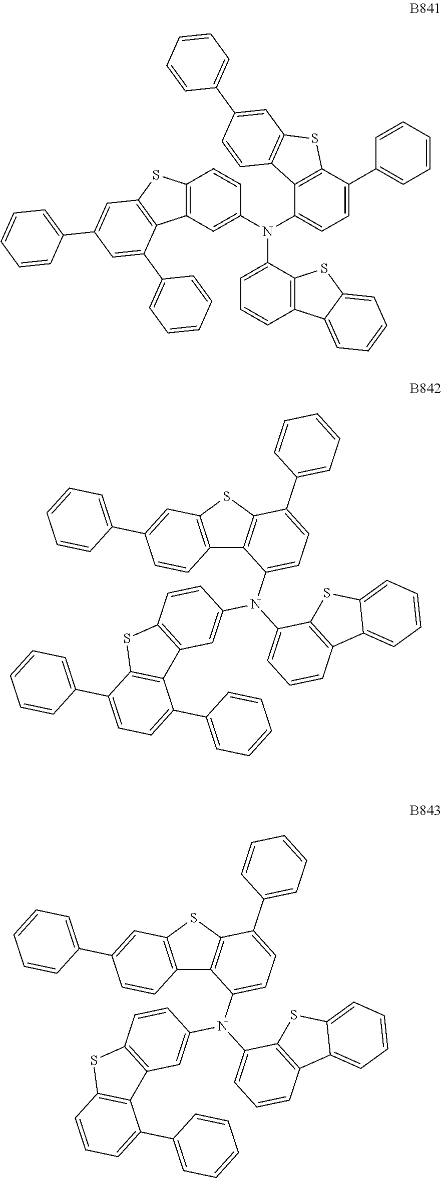 [SMILES: c1ccc(-c2ccc3c(c2)sc2c(-c4ccccc4)ccc(N(c4ccc5sc6cc(-c7ccccc7)cc(-c7ccccc7)c6c5c4)c4cccc5c4sc4ccccc45)c23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5c4sc4ccccc45)c23)cc1 ; c1ccc(-c2ccc3c(c2)sc2c(-c4ccccc4)ccc(N(c4ccc5sc6cccc(-c7ccccc7)c6c5c4)c4cccc5c4sc4ccccc45)c23)cc1]

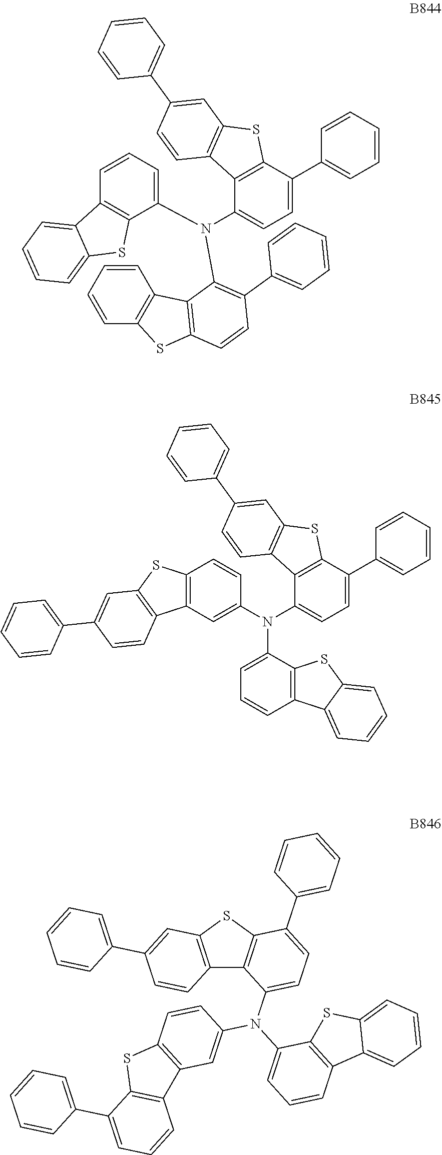 [SMILES: c1ccc(-c2ccc3c(c2)sc2c(-c4ccccc4)ccc(N(c4cccc5c4sc4ccccc45)c4c(-c5ccccc5)ccc5sc6ccccc6c45)c23)cc1 ; c1ccc(-c2ccc3c(c2)sc2ccc(N(c4cccc5c4sc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5c4sc4ccccc45)c23)cc1]

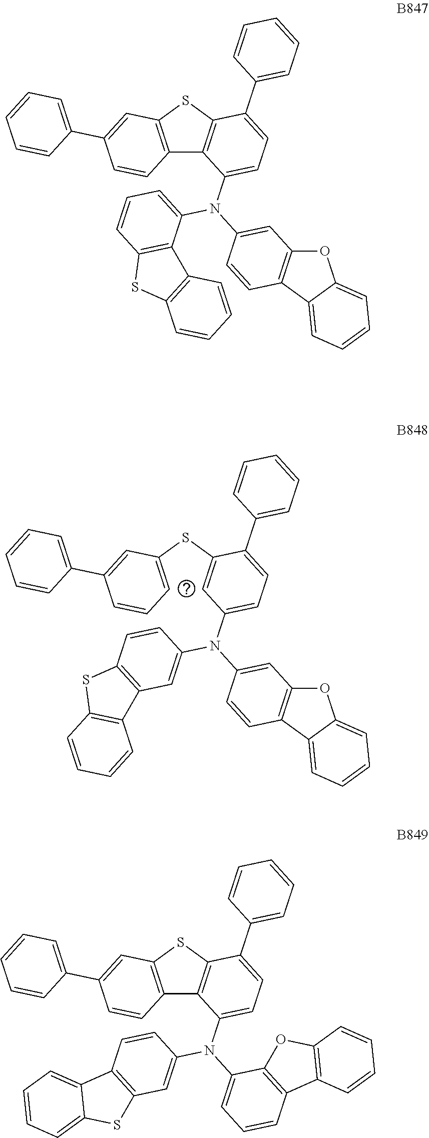 [SMILES: c1ccc(-c2ccc3c(c2)sc2c(-c4ccccc4)ccc(N(c4ccc5c(c4)oc4ccccc45)c4cccc5sc6ccccc6c45)c23)cc1 ; c1ccc(-c2cccc(Sc3cc(N(c4ccc5c(c4)oc4ccccc45)c4ccc5sc6ccccc6c5c4)ccc3-c3ccccc3)c2)cc1 ; c1ccc(-c2ccc3c(c2)sc2c(-c4ccccc4)ccc(N(c4ccc5c(c4)sc4ccccc45)c4cccc5c4oc4ccccc45)c23)cc1]

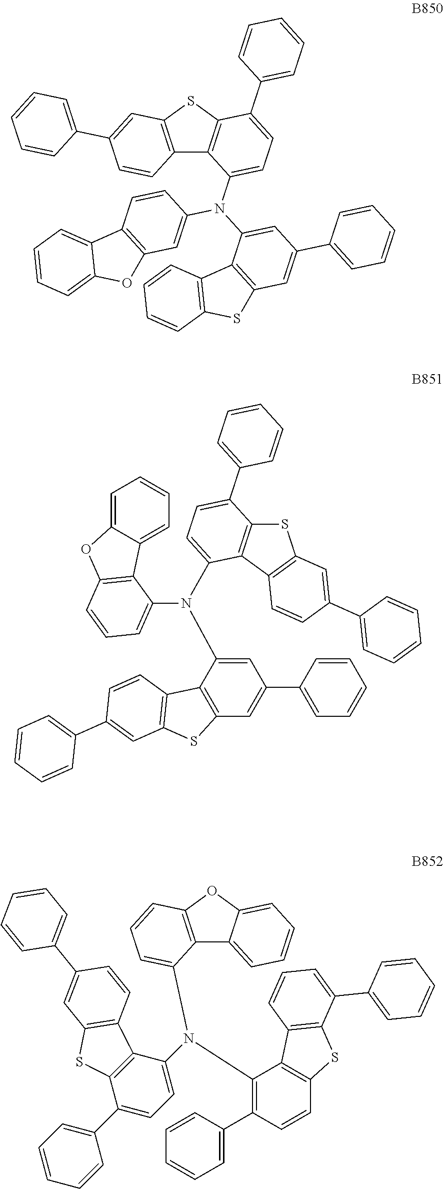 [SMILES: c1ccc(-c2ccc3c(c2)sc2c(-c4ccccc4)ccc(N(c4ccc5c(c4)oc4ccccc45)c4cc(-c5ccccc5)cc5sc6ccccc6c45)c23)cc1 ; c1ccc(-c2ccc3c(c2)sc2cc(-c4ccccc4)cc(N(c4cccc5oc6ccccc6c45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5oc6ccccc6c45)c4c(-c5ccccc5)ccc5sc6c(-c7ccccc7)cccc6c45)c23)cc1]

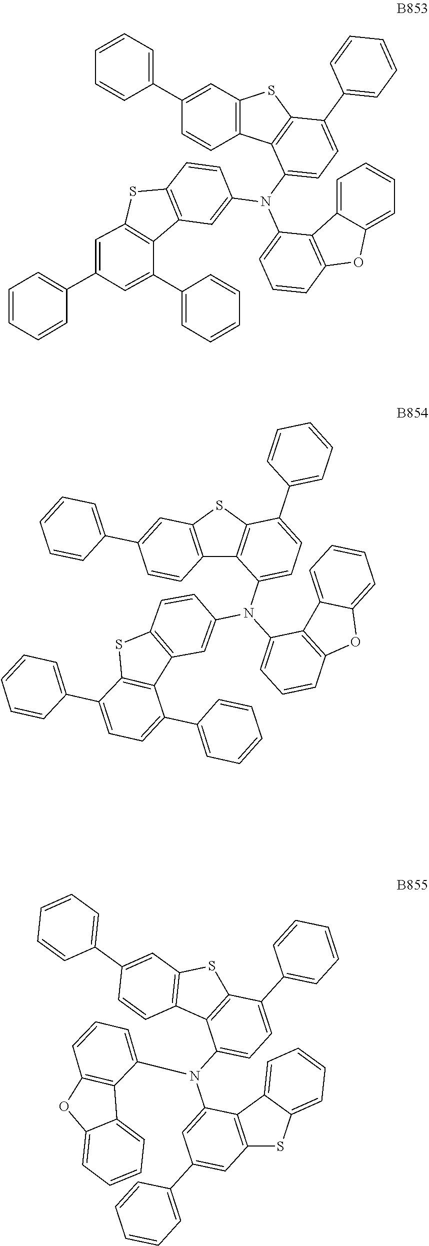 [SMILES: c1ccc(-c2ccc3c(c2)sc2c(-c4ccccc4)ccc(N(c4ccc5sc6cc(-c7ccccc7)cc(-c7ccccc7)c6c5c4)c4cccc5oc6ccccc6c45)c23)cc1 ; c1ccc(-c2ccc3c(c2)sc2c(-c4ccccc4)ccc(N(c4ccc5sc6c(-c7ccccc7)ccc(-c7ccccc7)c6c5c4)c4cccc5oc6ccccc6c45)c23)cc1 ; c1ccc(-c2ccc3c(c2)sc2c(-c4ccccc4)ccc(N(c4cccc5oc6ccccc6c45)c4cc(-c5ccccc5)cc5sc6ccccc6c45)c23)cc1]

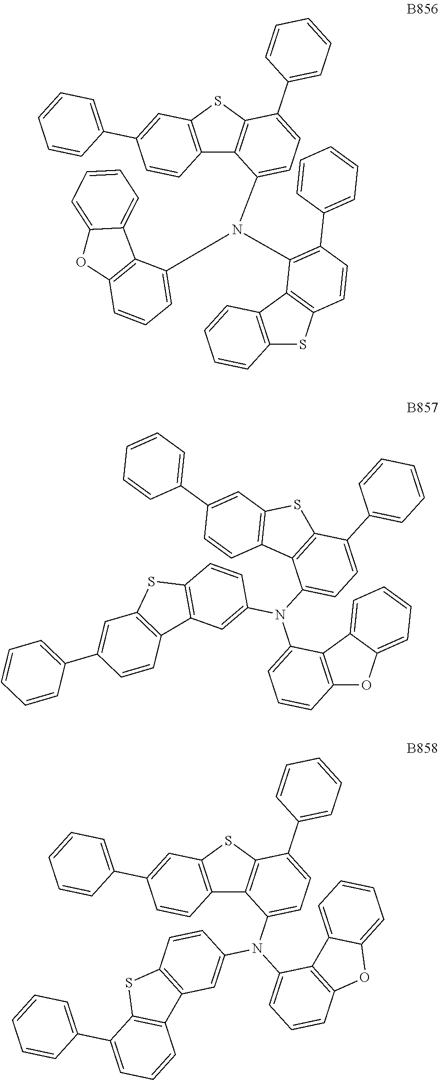 [SMILES: c1ccc(-c2ccc3c(c2)sc2c(-c4ccccc4)ccc(N(c4cccc5oc6ccccc6c45)c4c(-c5ccccc5)ccc5sc6ccccc6c45)c23)cc1 ; c1ccc(-c2ccc3c(c2)sc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5oc6ccccc6c45)c23)cc1]

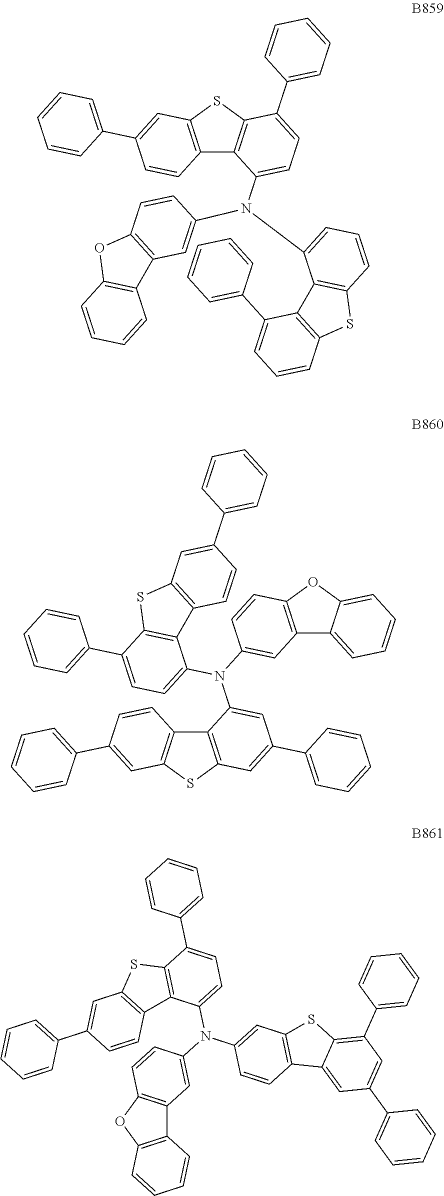 [SMILES: c1ccc(-c2ccc3c(c2)sc2c(-c4ccccc4)ccc(N(c4ccc5oc6ccccc6c5c4)c4cccc5sc6cccc(-c7ccccc7)c6c45)c23)cc1 ; c1ccc(-c2ccc3c(c2)sc2cc(-c4ccccc4)cc(N(c4ccc5oc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)sc4c(-c6ccccc6)cc(-c6ccccc6)cc45)c4ccc5oc6ccccc6c5c4)c23)cc1]

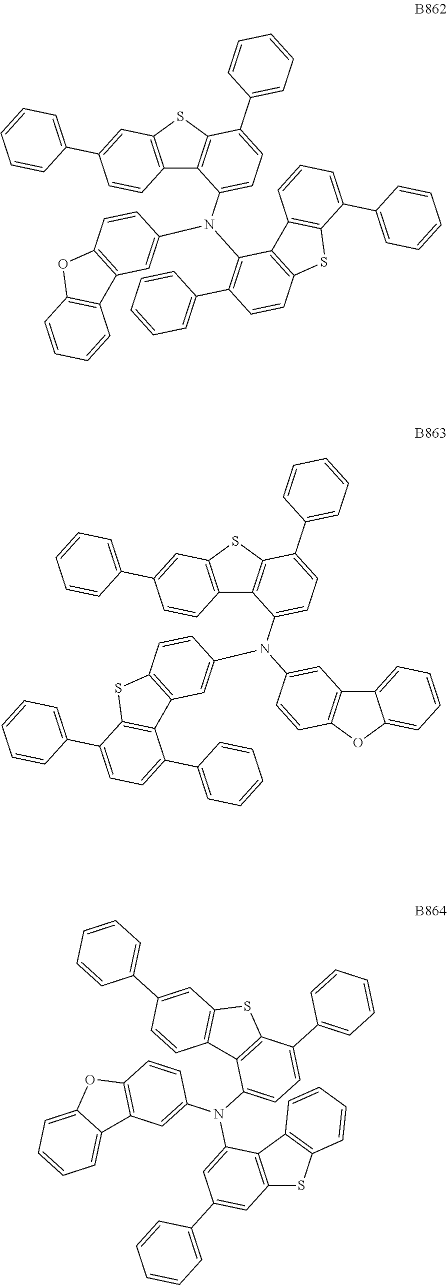 [SMILES: c1ccc(-c2ccc3c(c2)sc2c(-c4ccccc4)ccc(N(c4ccc5oc6ccccc6c5c4)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5oc6ccccc6c5c4)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5oc6ccccc6c5c4)c4cc(-c5ccccc5)cc5sc6ccccc6c45)c23)cc1]

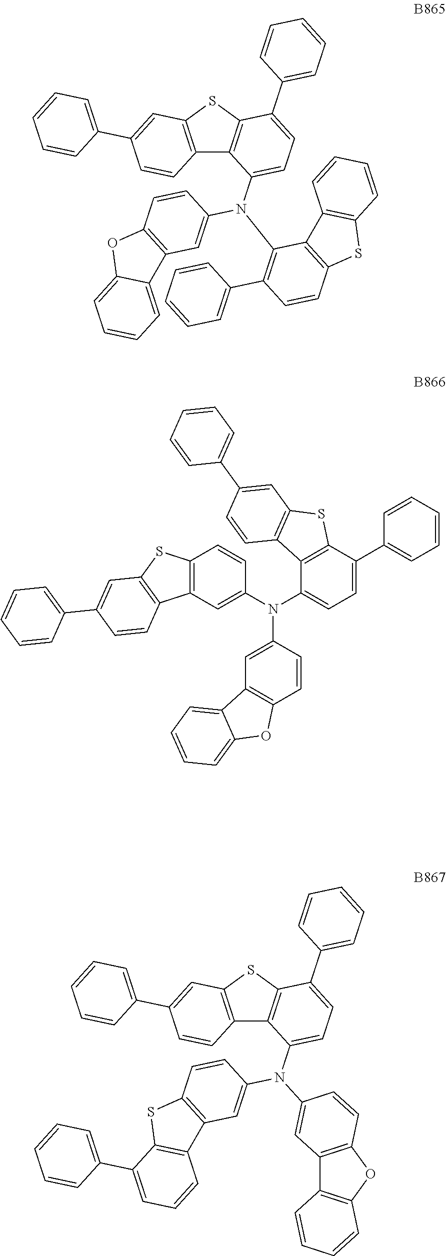 [SMILES: c1ccc(-c2ccc3c(c2)sc2c(-c4ccccc4)ccc(N(c4ccc5oc6ccccc6c5c4)c4c(-c5ccccc5)ccc5sc6ccccc6c45)c23)cc1 ; c1ccc(-c2ccc3c(c2)sc2ccc(N(c4ccc5oc6ccccc6c5c4)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6ccccc6c5c4)c4ccc5sc6c(-c7ccccc7)cccc6c5c4)c23)cc1]

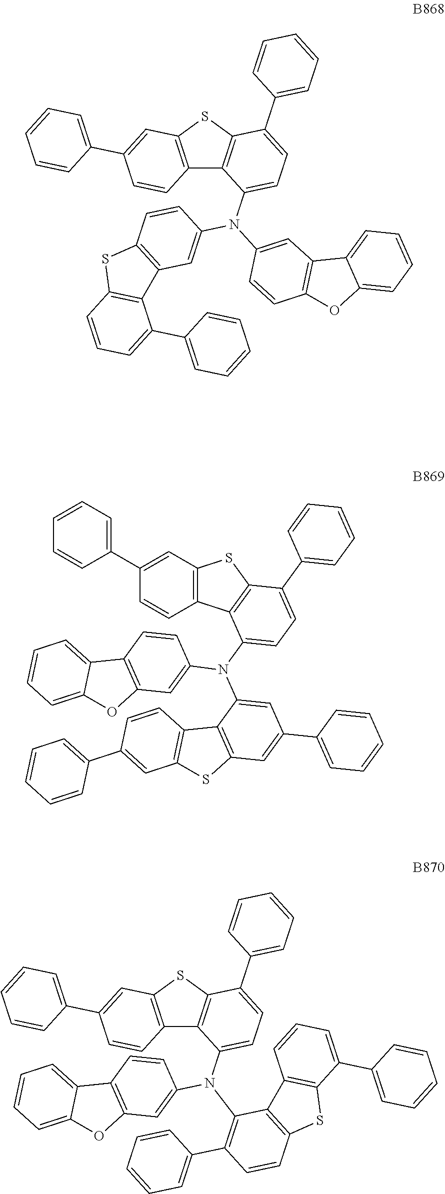 [SMILES: c1ccc(-c2ccc3c(c2)sc2c(-c4ccccc4)ccc(N(c4ccc5oc6ccccc6c5c4)c4ccc5sc6cccc(-c7ccccc7)c6c5c4)c23)cc1 ; c1ccc(-c2ccc3c(c2)sc2cc(-c4ccccc4)cc(N(c4ccc5c(c4)oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)oc4ccccc45)c4c(-c5ccccc5)ccc5sc6c(-c7ccccc7)cccc6c45)c23)cc1]

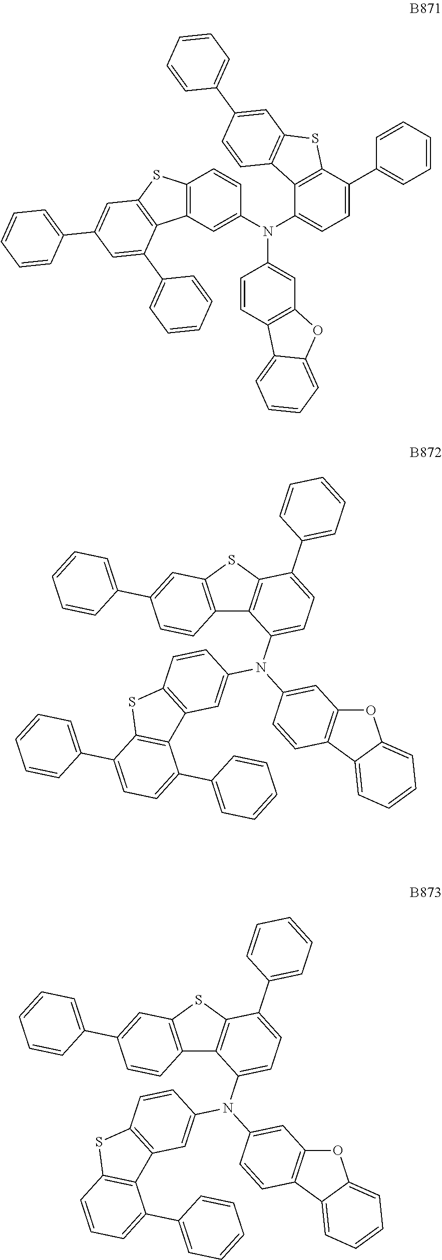 [SMILES: c1ccc(-c2ccc3c(c2)sc2c(-c4ccccc4)ccc(N(c4ccc5c(c4)oc4ccccc45)c4ccc5sc6cc(-c7ccccc7)cc(-c7ccccc7)c6c5c4)c23)cc1 ; c1ccc(-c2ccc3c(c2)sc2c(-c4ccccc4)ccc(N(c4ccc5c(c4)oc4ccccc45)c4ccc5sc6c(-c7ccccc7)ccc(-c7ccccc7)c6c5c4)c23)cc1 ; c1ccc(-c2ccc3c(c2)sc2c(-c4ccccc4)ccc(N(c4ccc5c(c4)oc4ccccc45)c4ccc5sc6cccc(-c7ccccc7)c6c5c4)c23)cc1]

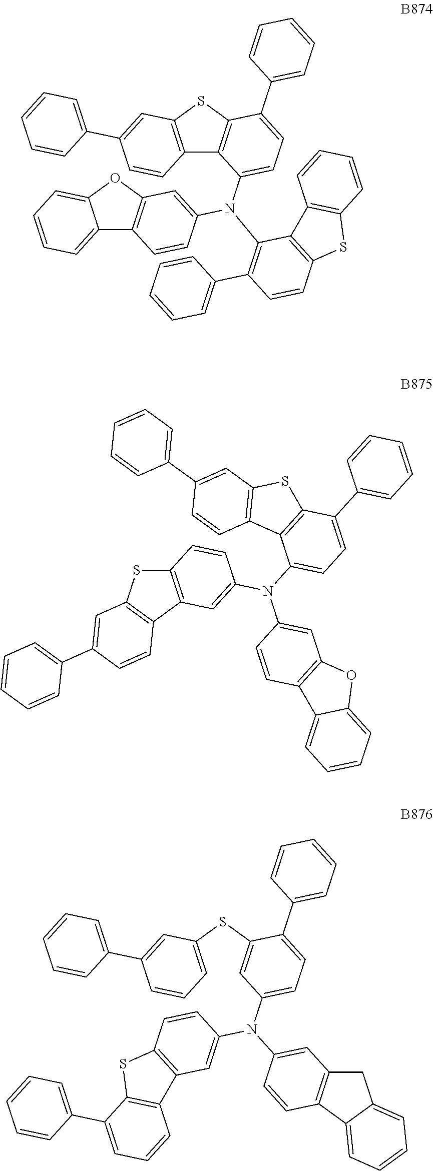 [SMILES: c1ccc(-c2ccc3c(c2)sc2c(-c4ccccc4)ccc(N(c4ccc5c(c4)oc4ccccc45)c4c(-c5ccccc5)ccc5sc6ccccc6c45)c23)cc1 ; c1ccc(-c2ccc3c(c2)sc2ccc(N(c4ccc5c(c4)oc4ccccc45)c4ccc(-c5ccccc5)c5sc6cc(-c7ccccc7)ccc6c45)cc23)cc1 ; c1ccc(-c2cccc(Sc3cc(N(c4ccc5c(c4)Cc4ccccc4-5)c4ccc5sc6c(-c7ccccc7)cccc6c5c4)ccc3-c3ccccc3)c2)cc1]

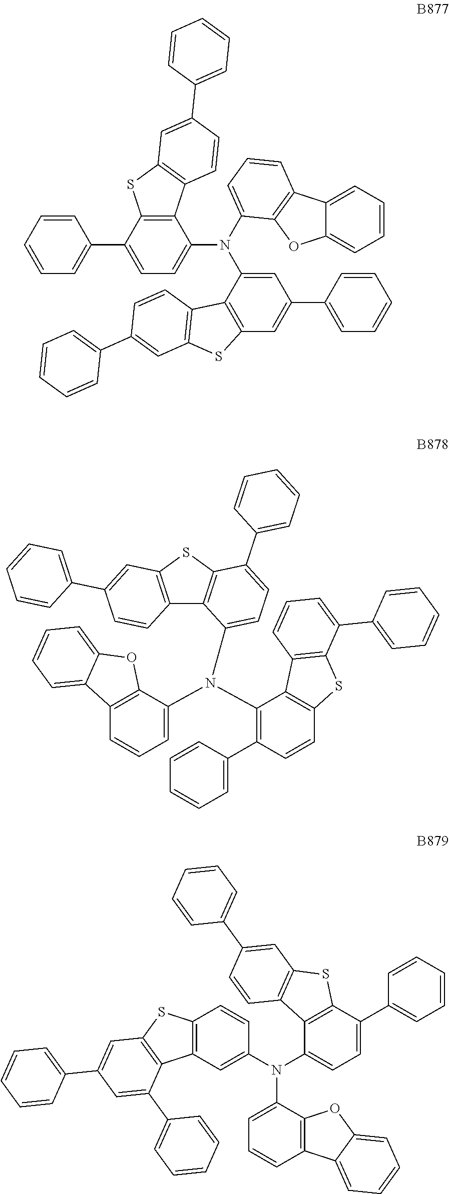 [SMILES: c1ccc(-c2ccc3c(c2)sc2cc(-c4ccccc4)cc(N(c4cccc5c4oc4ccccc45)c4ccc(-c5ccccc5)c5sc6cc(-c7ccccc7)ccc6c45)c23)cc1 ; c1ccc(-c2ccc3c(c2)sc2c(-c4ccccc4)ccc(N(c4cccc5c4oc4ccccc45)c4c(-c5ccccc5)ccc5sc6c(-c7ccccc7)cccc6c45)c23)cc1 ; c1ccc(-c2ccc3c(c2)sc2c(-c4ccccc4)ccc(N(c4ccc5sc6cc(-c7ccccc7)cc(-c7ccccc7)c6c5c4)c4cccc5c4oc4ccccc45)c23)cc1]

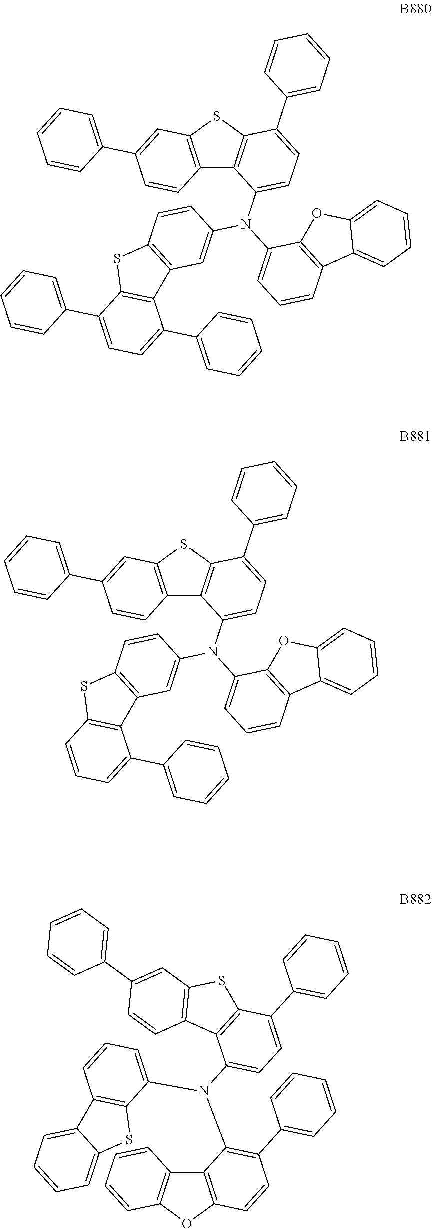 [SMILES: c1ccc(-c2ccc3c(c2)sc2c(-c4ccccc4)ccc(N(c4ccc5sc6c(-c7ccccc7)ccc(-c7ccccc7)c6c5c4)c4cccc5c4oc4ccccc45)c23)cc1 ; c1ccc(-c2ccc3c(c2)sc2c(-c4ccccc4)ccc(N(c4ccc5sc6cccc(-c7ccccc7)c6c5c4)c4cccc5c4oc4ccccc45)c23)cc1 ; c1ccc(-c2ccc3c(c2)sc2c(-c4ccccc4)ccc(N(c4cccc5c4sc4ccccc45)c4c(-c5ccccc5)ccc5oc6ccccc6c45)c23)cc1]

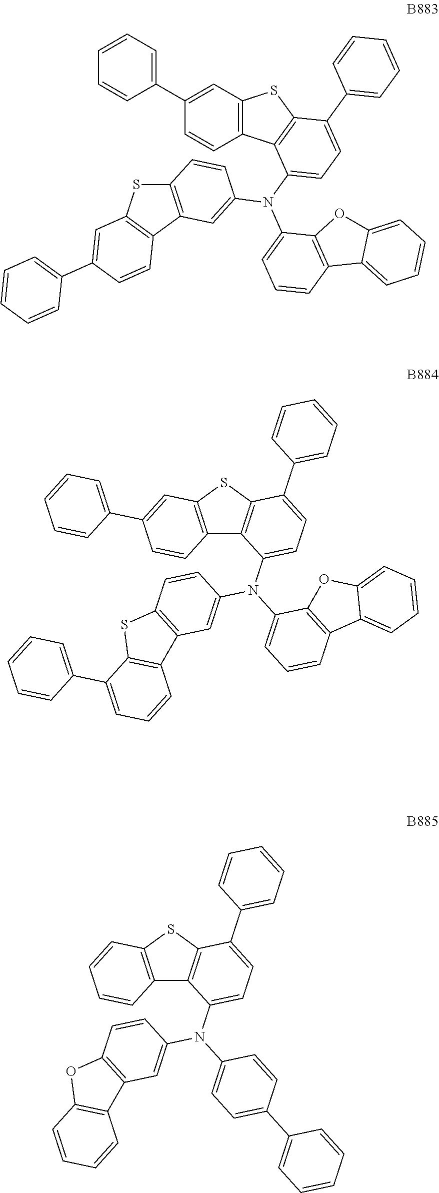 [SMILES: c1ccc(-c2ccc3c(c2)sc2ccc(N(c4cccc5c4oc4ccccc45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5sc6c(-c7ccccc7)cccc6c5c4)c4cccc5c4oc4ccccc45)c23)cc1 ; c1ccc(-c2ccc(N(c3ccc4oc5ccccc5c4c3)c3ccc(-c4ccccc4)c4sc5ccccc5c34)cc2)cc1]

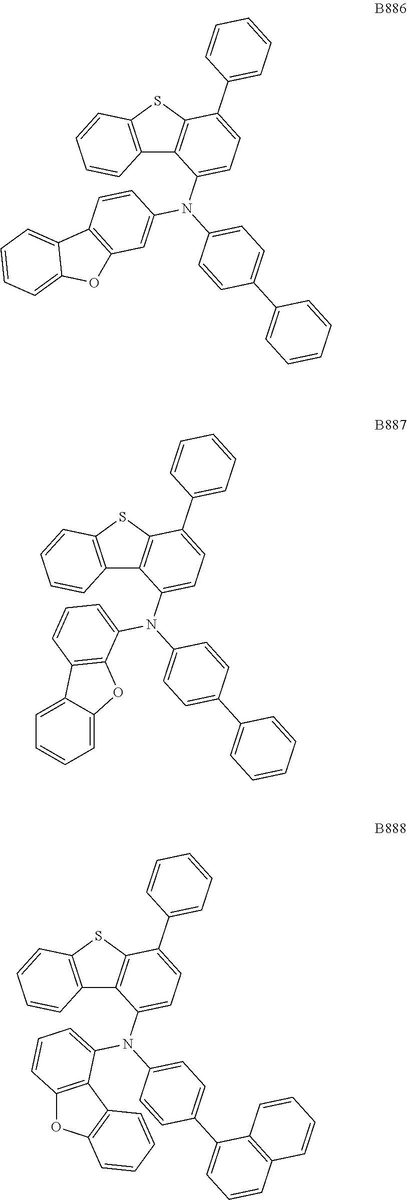 [SMILES: c1ccc(-c2ccc(N(c3ccc4c(c3)oc3ccccc34)c3ccc(-c4ccccc4)c4sc5ccccc5c34)cc2)cc1 ; c1ccc(-c2ccc(N(c3cccc4c3oc3ccccc34)c3ccc(-c4ccccc4)c4sc5ccccc5c34)cc2)cc1 ; c1ccc(-c2ccc(N(c3ccc(-c4cccc5ccccc45)cc3)c3cccc4oc5ccccc5c34)c3c2sc2ccccc23)cc1]

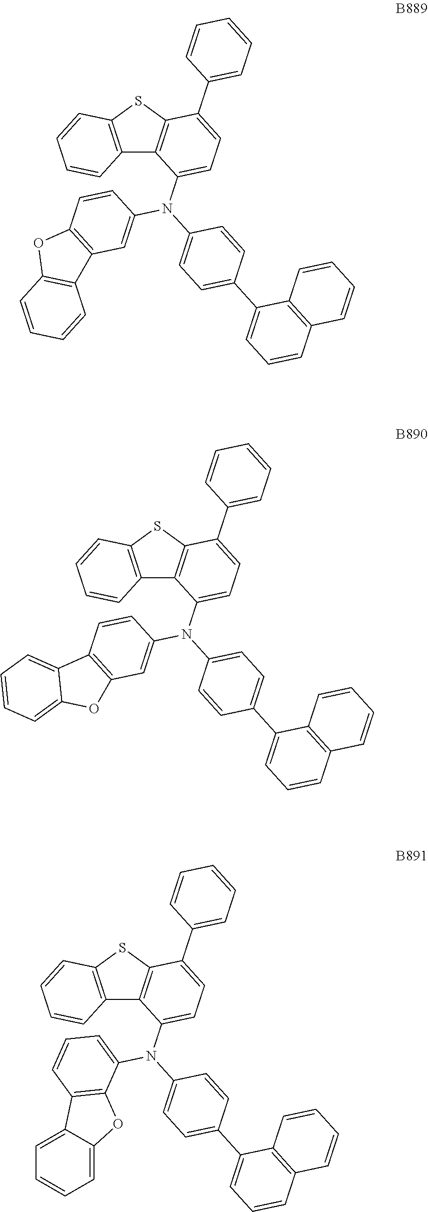 [SMILES: c1ccc(-c2ccc(N(c3ccc(-c4cccc5ccccc45)cc3)c3ccc4oc5ccccc5c4c3)c3c2sc2ccccc23)cc1 ; c1ccc(-c2ccc(N(c3ccc(-c4cccc5ccccc45)cc3)c3ccc4c(c3)oc3ccccc34)c3c2sc2ccccc23)cc1 ; c1ccc(-c2ccc(N(c3ccc(-c4cccc5ccccc45)cc3)c3cccc4c3oc3ccccc34)c3c2sc2ccccc23)cc1]

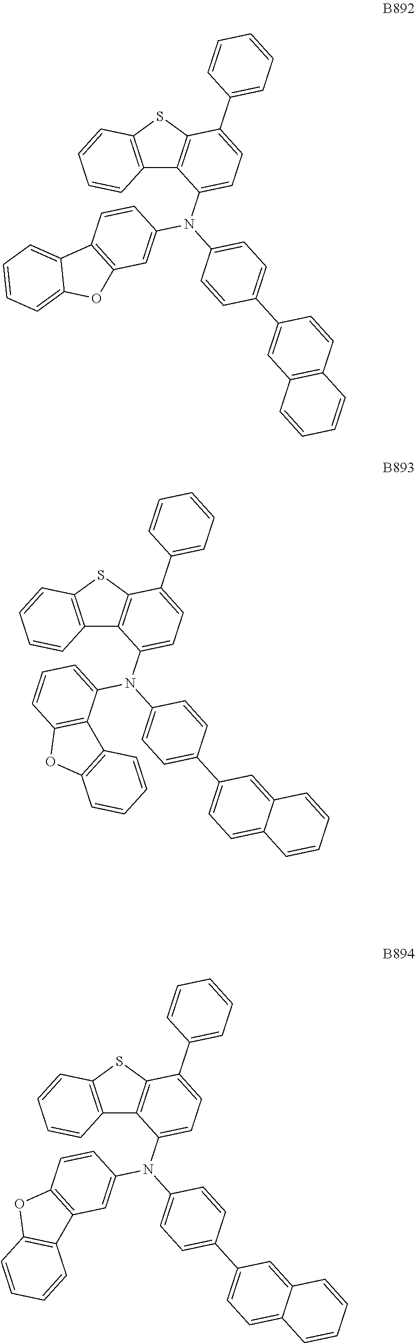 [SMILES: c1ccc(-c2ccc(N(c3ccc(-c4ccc5ccccc5c4)cc3)c3ccc4c(c3)oc3ccccc34)c3c2sc2ccccc23)cc1 ; c1ccc(-c2ccc(N(c3ccc(-c4ccc5ccccc5c4)cc3)c3cccc4oc5ccccc5c34)c3c2sc2ccccc23)cc1 ; c1ccc(-c2ccc(N(c3ccc(-c4ccc5ccccc5c4)cc3)c3ccc4oc5ccccc5c4c3)c3c2sc2ccccc23)cc1]

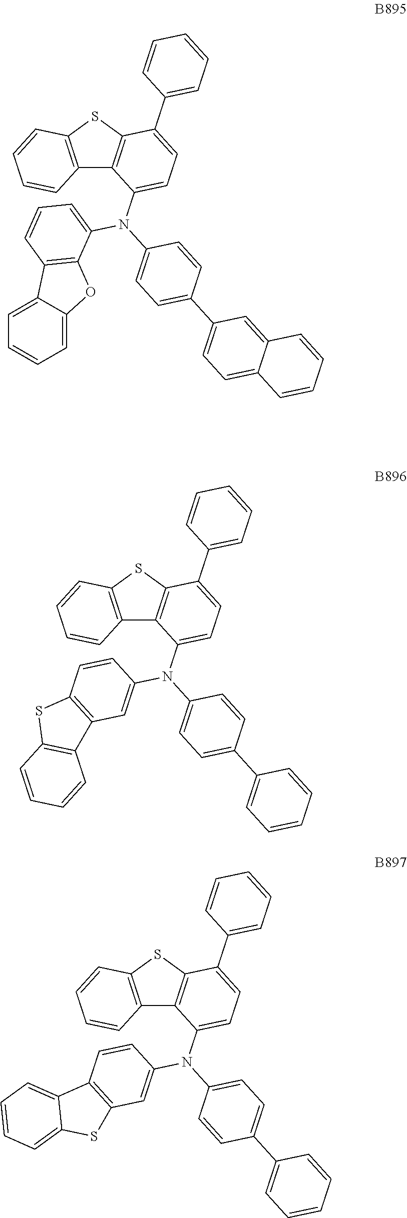

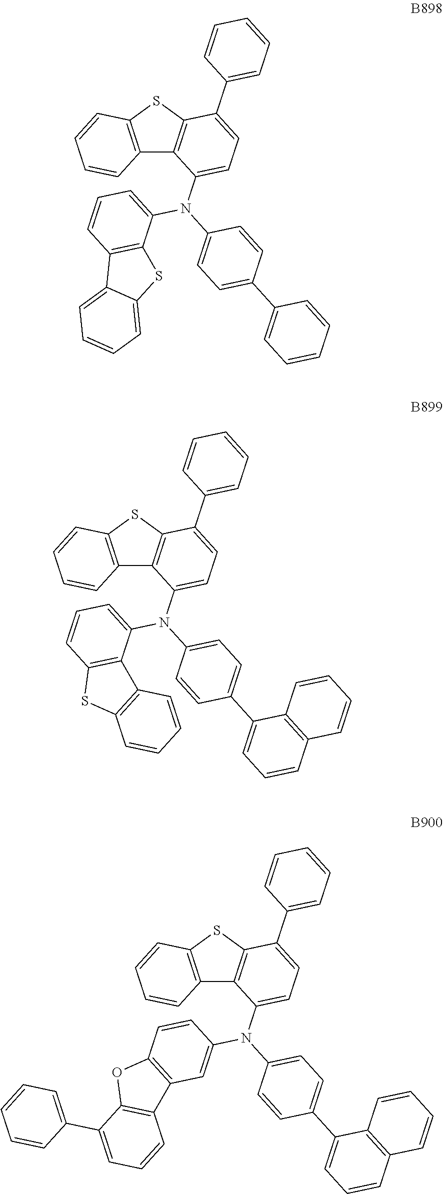 [SMILES: c1ccc(-c2ccc(N(c3cccc4c3sc3ccccc34)c3ccc(-c4ccccc4)c4sc5ccccc5c34)cc2)cc1 ; c1ccc(-c2ccc(N(c3ccc(-c4cccc5ccccc45)cc3)c3cccc4sc5ccccc5c34)c3c2sc2ccccc23)cc1 ; c1ccc(-c2cccc3c2oc2ccc(N(c4ccc(-c5cccc6ccccc56)cc4)c4ccc(-c5ccccc5)c5sc6ccccc6c45)cc23)cc1]

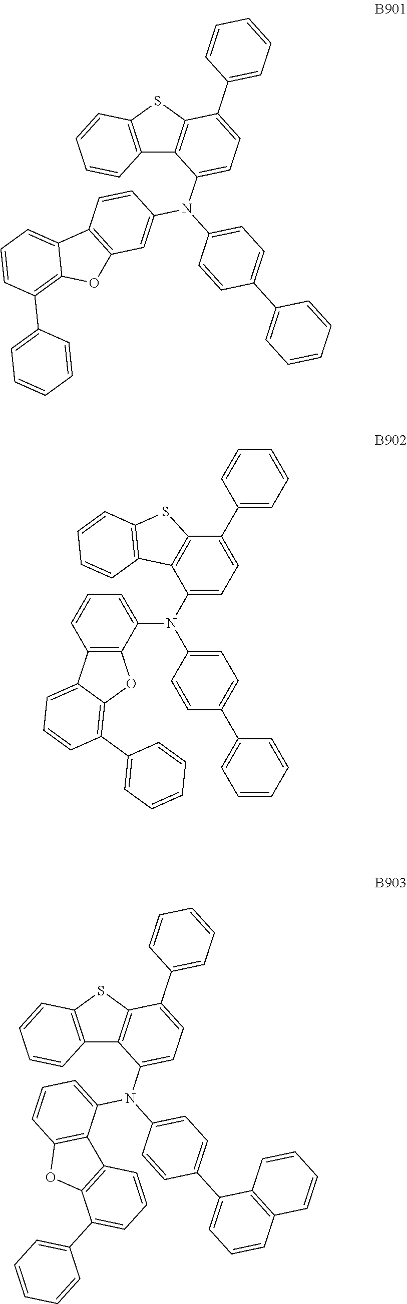 [SMILES: c1ccc(-c2ccc(N(c3ccc4c(c3)oc3c(-c5ccccc5)cccc34)c3ccc(-c4ccccc4)c4sc5ccccc5c34)cc2)cc1 ; c1ccc(-c2ccc(N(c3cccc4c3oc3c(-c5ccccc5)cccc34)c3ccc(-c4ccccc4)c4sc5ccccc5c34)cc2)cc1 ; c1ccc(-c2cccc3c2oc2cccc(N(c4ccc(-c5cccc6ccccc56)cc4)c4ccc(-c5ccccc5)c5sc6ccccc6c45)c23)cc1]

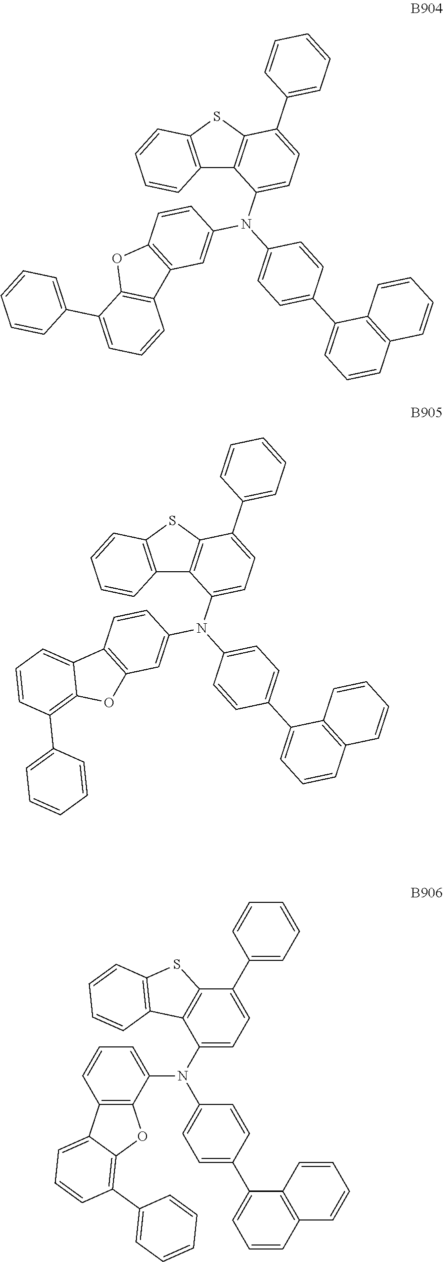 [SMILES: c1ccc(-c2cccc3c2oc2ccc(N(c4ccc(-c5cccc6ccccc56)cc4)c4ccc(-c5ccccc5)c5sc6ccccc6c45)cc23)cc1 ; c1ccc(-c2cccc3c2oc2cc(N(c4ccc(-c5cccc6ccccc56)cc4)c4ccc(-c5ccccc5)c5sc6ccccc6c45)ccc23)cc1 ; c1ccc(-c2cccc3c2oc2c(N(c4ccc(-c5cccc6ccccc56)cc4)c4ccc(-c5ccccc5)c5sc6ccccc6c45)cccc23)cc1]

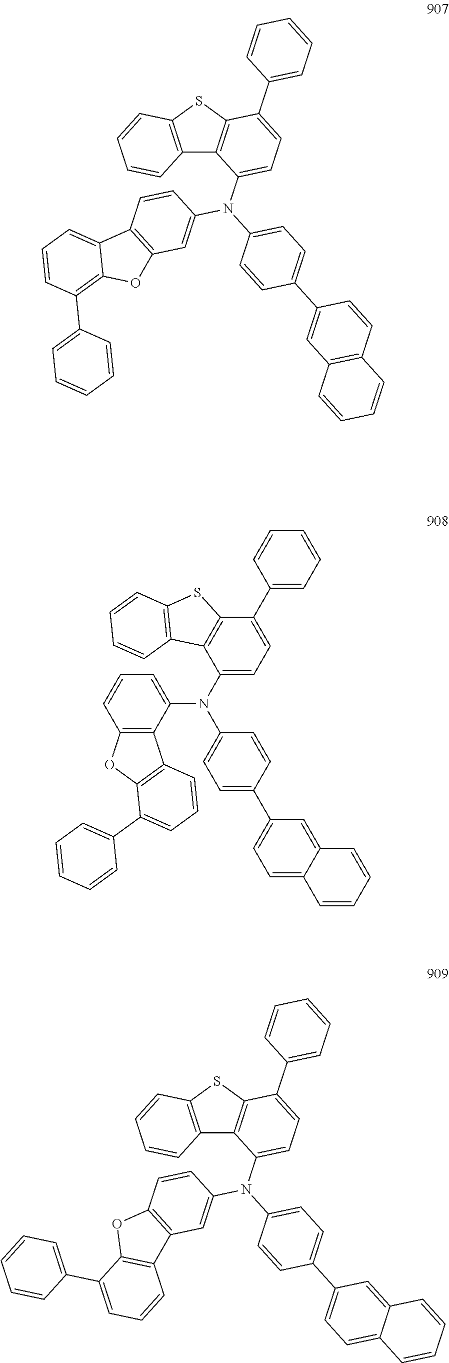 [SMILES: c1ccc(-c2cccc3c2oc2cc(N(c4ccc(-c5ccc6ccccc6c5)cc4)c4ccc(-c5ccccc5)c5sc6ccccc6c45)ccc23)cc1 ; c1ccc(-c2cccc3c2oc2cccc(N(c4ccc(-c5ccc6ccccc6c5)cc4)c4ccc(-c5ccccc5)c5sc6ccccc6c45)c23)cc1 ; c1ccc(-c2cccc3c2oc2ccc(N(c4ccc(-c5ccc6ccccc6c5)cc4)c4ccc(-c5ccccc5)c5sc6ccccc6c45)cc23)cc1]

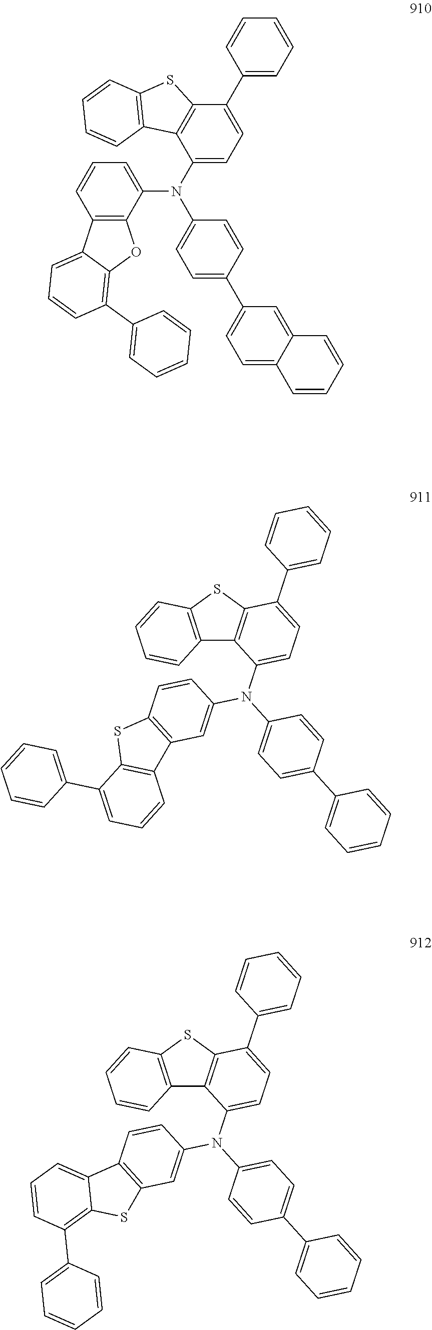 [SMILES: c1ccc(-c2cccc3c2oc2c(N(c4ccc(-c5ccc6ccccc6c5)cc4)c4ccc(-c5ccccc5)c5sc6ccccc6c45)cccc23)cc1 ; c1ccc(-c2ccc(N(c3ccc4sc5c(-c6ccccc6)cccc5c4c3)c3ccc(-c4ccccc4)c4sc5ccccc5c34)cc2)cc1 ; c1ccc(-c2ccc(N(c3ccc4c(c3)sc3c(-c5ccccc5)cccc34)c3ccc(-c4ccccc4)c4sc5ccccc5c34)cc2)cc1]

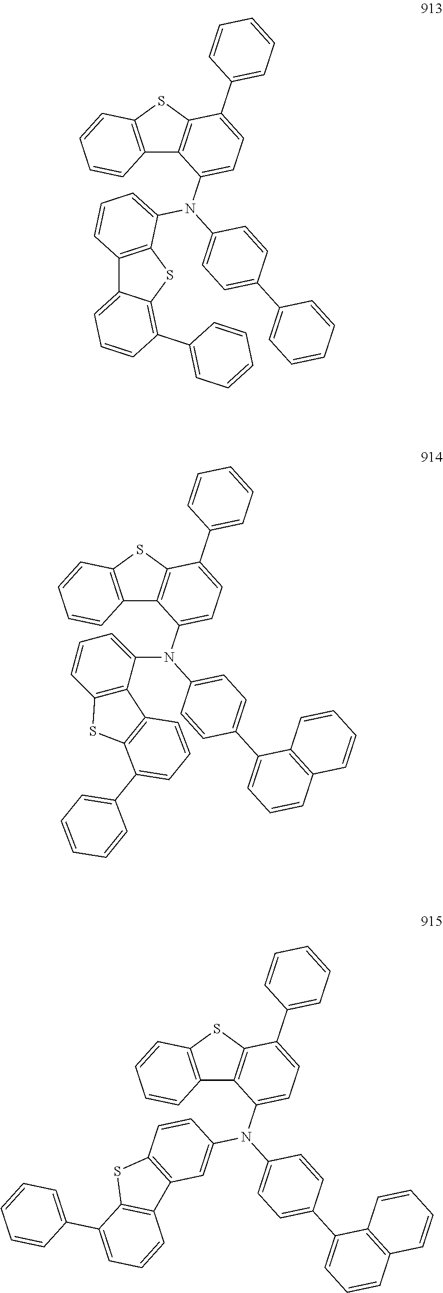 [SMILES: c1ccc(-c2ccc(N(c3cccc4c3sc3c(-c5ccccc5)cccc34)c3ccc(-c4ccccc4)c4sc5ccccc5c34)cc2)cc1 ; c1ccc(-c2cccc3c2sc2cccc(N(c4ccc(-c5cccc6ccccc56)cc4)c4ccc(-c5ccccc5)c5sc6ccccc6c45)c23)cc1 ; c1ccc(-c2cccc3c2sc2ccc(N(c4ccc(-c5cccc6ccccc56)cc4)c4ccc(-c5ccccc5)c5sc6ccccc6c45)cc23)cc1]

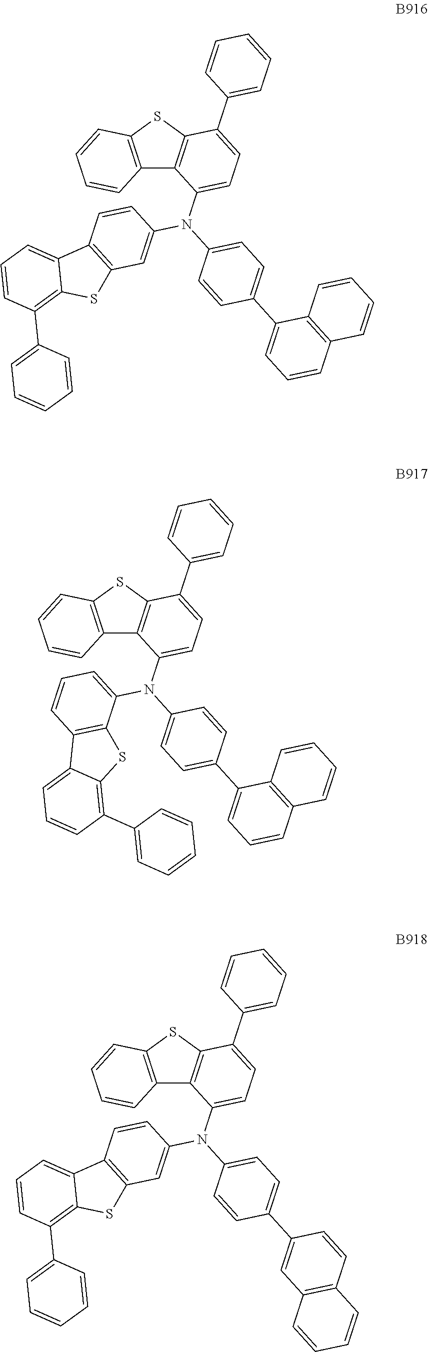 [SMILES: c1ccc(-c2cccc3c2sc2cc(N(c4ccc(-c5cccc6ccccc56)cc4)c4ccc(-c5ccccc5)c5sc6ccccc6c45)ccc23)cc1 ; c1ccc(-c2cccc3c2sc2c(N(c4ccc(-c5cccc6ccccc56)cc4)c4ccc(-c5ccccc5)c5sc6ccccc6c45)cccc23)cc1 ; c1ccc(-c2cccc3c2sc2cc(N(c4ccc(-c5ccc6ccccc6c5)cc4)c4ccc(-c5ccccc5)c5sc6ccccc6c45)ccc23)cc1]

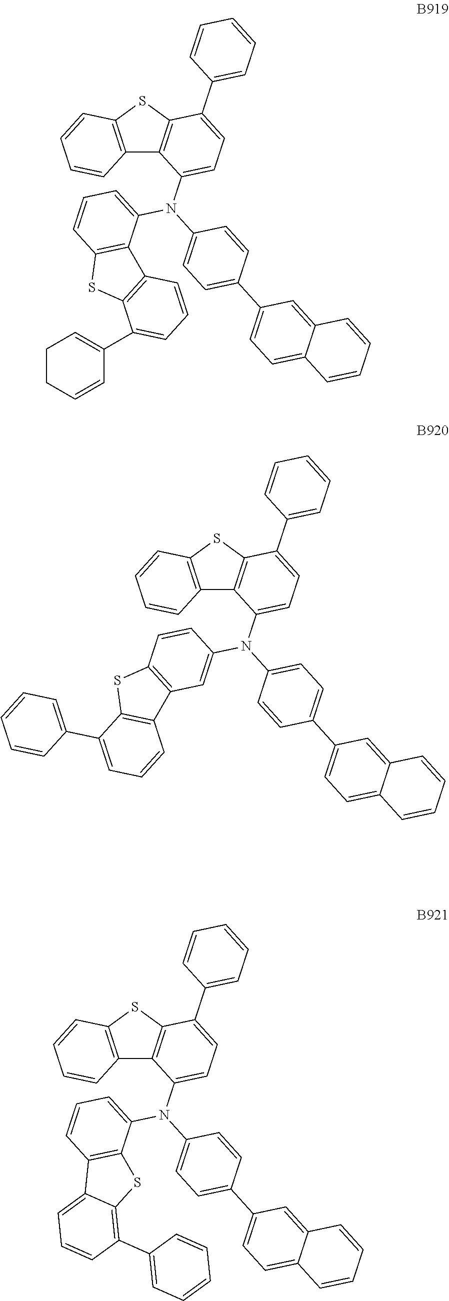 [SMILES: C1=CC(c2cccc3c2sc2cccc(N(c4ccc(-c5ccc6ccccc6c5)cc4)c4ccc(-c5ccccc5)c5sc6ccccc6c45)c23)=CCC1 ; c1ccc(-c2cccc3c2sc2ccc(N(c4ccc(-c5ccc6ccccc6c5)cc4)c4ccc(-c5ccccc5)c5sc6ccccc6c45)cc23)cc1 ; c1ccc(-c2cccc3c2sc2c(N(c4ccc(-c5ccc6ccccc6c5)cc4)c4ccc(-c5ccccc5)c5sc6ccccc6c45)cccc23)cc1]

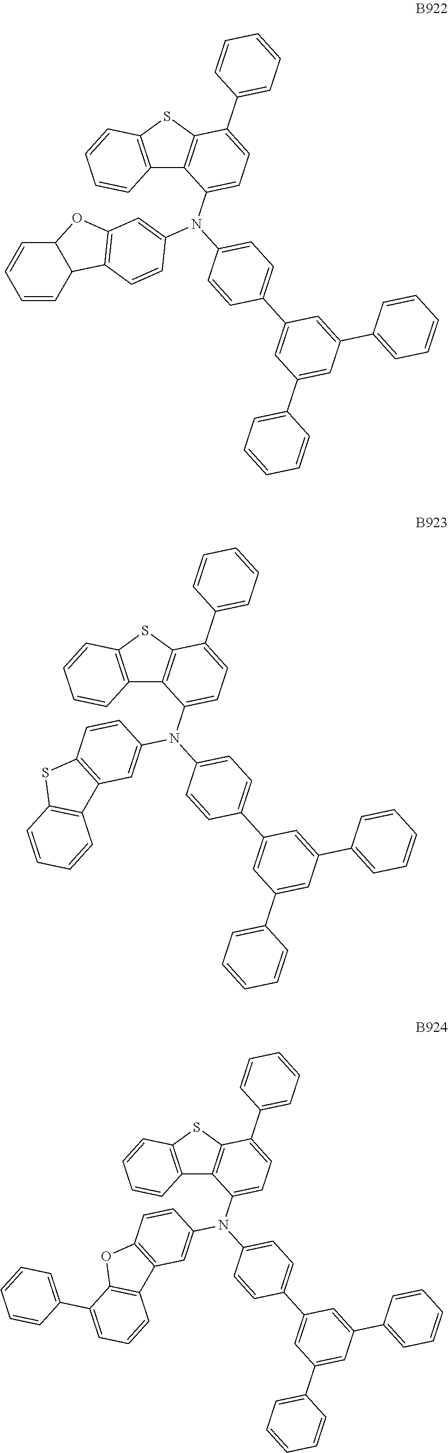 [SMILES: C1=CC2Oc3cc(N(c4ccc(-c5cc(-c6ccccc6)cc(-c6ccccc6)c5)cc4)c4ccc(-c5ccccc5)c5sc6ccccc6c45)ccc3C2C=C1 ; c1ccc(-c2cc(-c3ccccc3)cc(-c3ccc(N(c4ccc5sc6ccccc6c5c4)c4ccc(-c5ccccc5)c5sc6ccccc6c45)cc3)c2)cc1 ; c1ccc(-c2cc(-c3ccccc3)cc(-c3ccc(N(c4ccc5oc6c(-c7ccccc7)cccc6c5c4)c4ccc(-c5ccccc5)c5sc6ccccc6c45)cc3)c2)cc1]

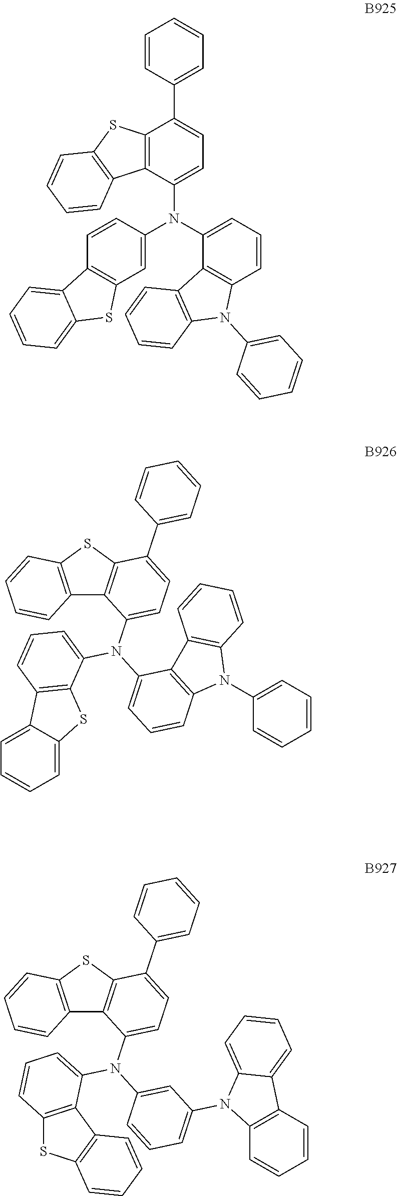 [SMILES: c1ccc(-c2ccc(N(c3ccc4c(c3)sc3ccccc34)c3cccc4c3c3ccccc3n4-c3ccccc3)c3c2sc2ccccc23)cc1 ; c1ccc(-c2ccc(N(c3cccc4c3sc3ccccc34)c3cccc4c3c3ccccc3n4-c3ccccc3)c3c2sc2ccccc23)cc1 ; c1ccc(-c2ccc(N(c3cccc(-n4c5ccccc5c5ccccc54)c3)c3cccc4sc5ccccc5c34)c3c2sc2ccccc23)cc1]

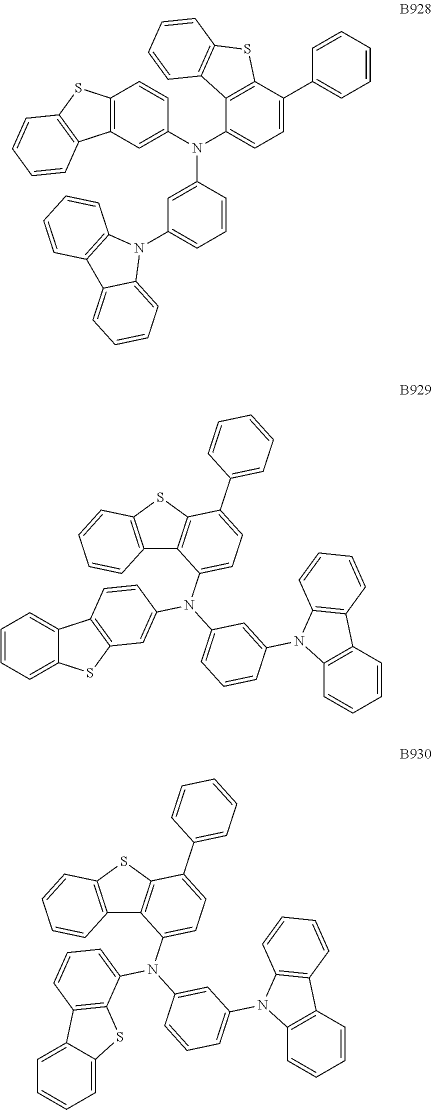 [SMILES: c1ccc(-c2ccc(N(c3cccc(-n4c5ccccc5c5ccccc54)c3)c3ccc4sc5ccccc5c4c3)c3c2sc2ccccc23)cc1 ; c1ccc(-c2ccc(N(c3cccc(-n4c5ccccc5c5ccccc54)c3)c3ccc4c(c3)sc3ccccc34)c3c2sc2ccccc23)cc1 ; c1ccc(-c2ccc(N(c3cccc(-n4c5ccccc5c5ccccc54)c3)c3cccc4c3sc3ccccc34)c3c2sc2ccccc23)cc1]

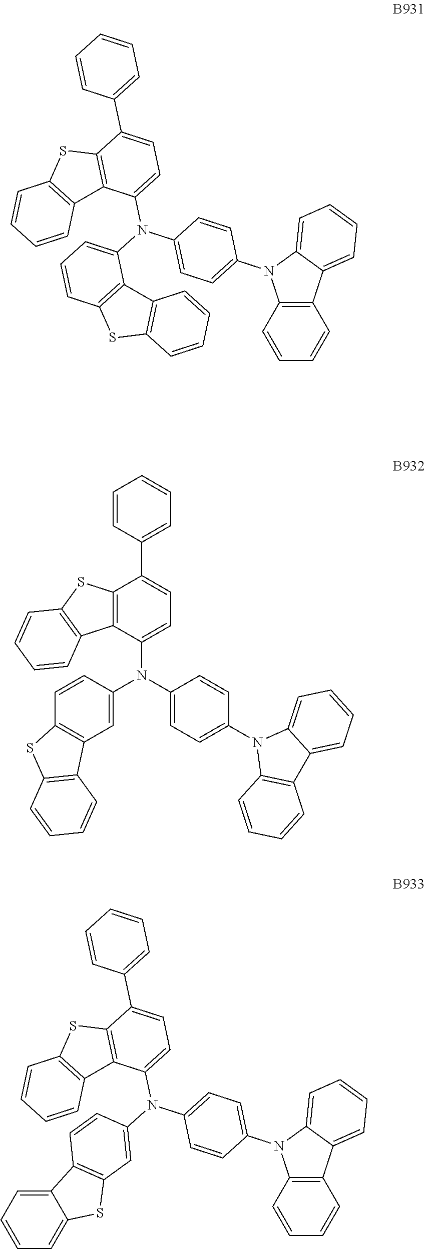 [SMILES: c1ccc(-c2ccc(N(c3ccc(-n4c5ccccc5c5ccccc54)cc3)c3cccc4sc5ccccc5c34)c3c2sc2ccccc23)cc1 ; c1ccc(-c2ccc(N(c3ccc(-n4c5ccccc5c5ccccc54)cc3)c3ccc4sc5ccccc5c4c3)c3c2sc2ccccc23)cc1 ; c1ccc(-c2ccc(N(c3ccc(-n4c5ccccc5c5ccccc54)cc3)c3ccc4c(c3)sc3ccccc34)c3c2sc2ccccc23)cc1]

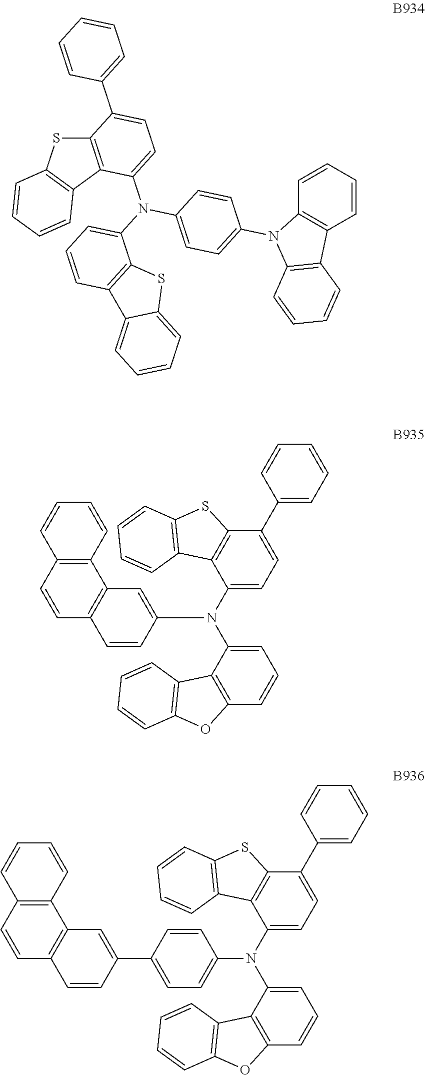 [SMILES: c1ccc(-c2ccc(N(c3ccc(-n4c5ccccc5c5ccccc54)cc3)c3cccc4c3sc3ccccc34)c3c2sc2ccccc23)cc1 ; c1ccc(-c2ccc(N(c3ccc4ccc5ccccc5c4c3)c3cccc4oc5ccccc5c34)c3c2sc2ccccc23)cc1 ; c1ccc(-c2ccc(N(c3ccc(-c4ccc5ccc6ccccc6c5c4)cc3)c3cccc4oc5ccccc5c34)c3c2sc2ccccc23)cc1]

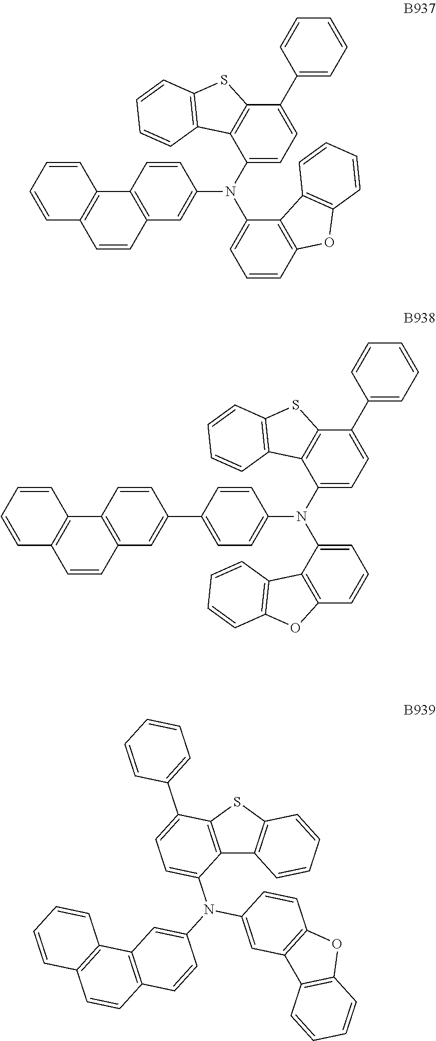 [SMILES: c1ccc(-c2ccc(N(c3ccc4c(ccc5ccccc54)c3)c3cccc4oc5ccccc5c34)c3c2sc2ccccc23)cc1 ; c1ccc(-c2ccc(N(c3ccc(-c4ccc5c(ccc6ccccc65)c4)cc3)c3cccc4oc5ccccc5c34)c3c2sc2ccccc23)cc1 ; c1ccc(-c2ccc(N(c3ccc4ccc5ccccc5c4c3)c3ccc4oc5ccccc5c4c3)c3c2sc2ccccc23)cc1]

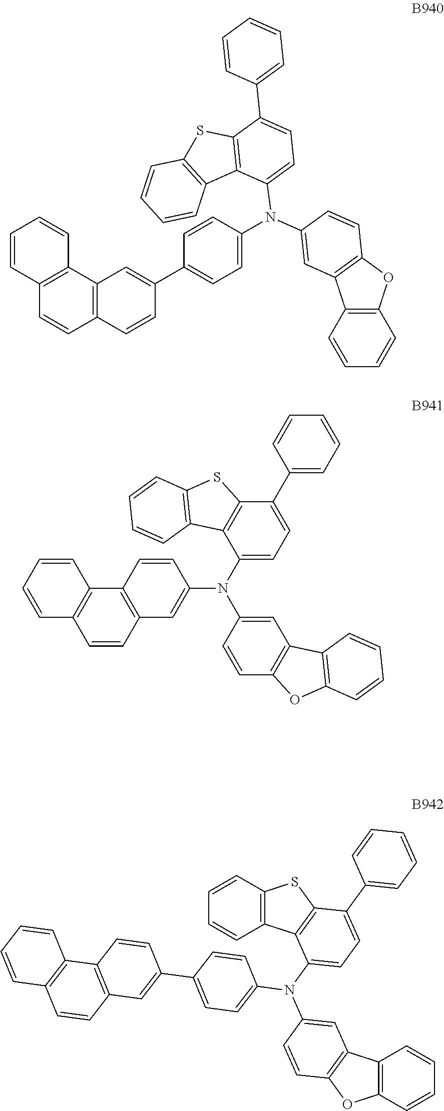 [SMILES: c1ccc(-c2ccc(N(c3ccc(-c4ccc5ccc6ccccc6c5c4)cc3)c3ccc4oc5ccccc5c4c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1 ; c1ccc(-c2ccc(N(c3ccc(-c4ccc5c(ccc6ccccc65)c4)cc3)c3ccc4oc5ccccc5c4c3)c3c2sc2ccccc23)cc1]

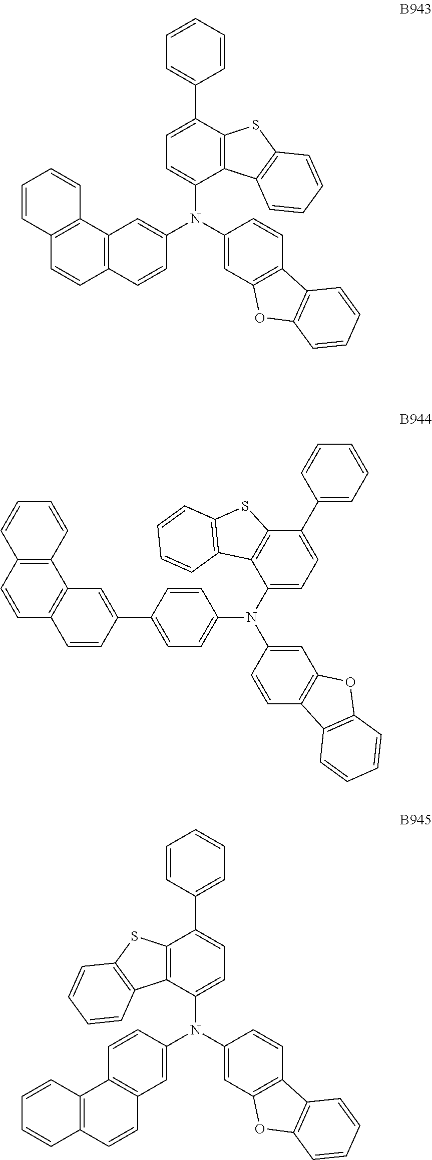 [SMILES: c1ccc(-c2ccc(N(c3ccc4c(c3)oc3ccccc34)c3ccc4ccc5ccccc5c4c3)c3c2sc2ccccc23)cc1 ; c1ccc(-c2ccc(N(c3ccc(-c4ccc5ccc6ccccc6c5c4)cc3)c3ccc4c(c3)oc3ccccc34)c3c2sc2ccccc23)cc1 ; c1ccc(-c2ccc(N(c3ccc4c(ccc5ccccc54)c3)c3ccc4c(c3)oc3ccccc34)c3c2sc2ccccc23)cc1]

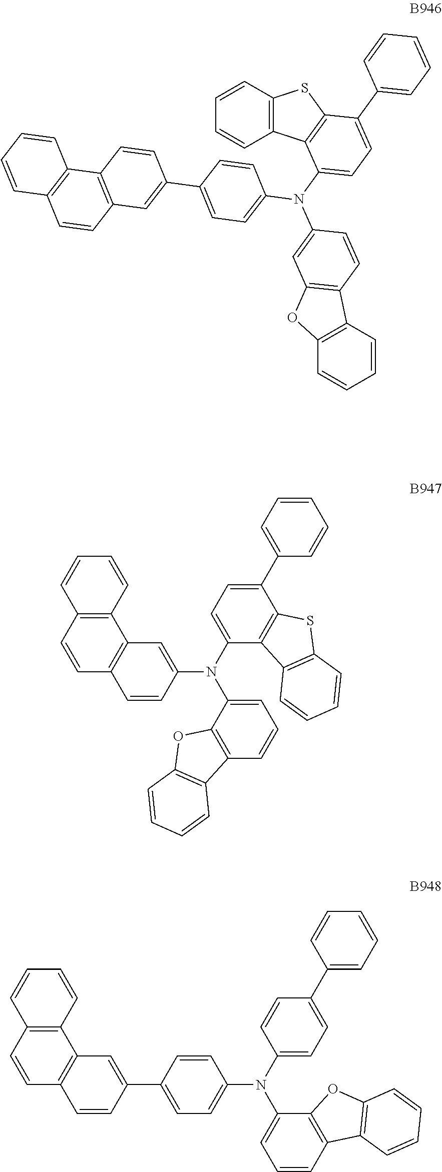 [SMILES: c1ccc(-c2ccc(N(c3ccc(-c4ccc5c(ccc6ccccc65)c4)cc3)c3ccc4c(c3)oc3ccccc34)c3c2sc2ccccc23)cc1 ; c1ccc(-c2ccc(N(c3ccc4ccc5ccccc5c4c3)c3cccc4c3oc3ccccc34)c3c2sc2ccccc23)cc1 ; c1ccc(-c2ccc(N(c3ccc(-c4ccc5ccc6ccccc6c5c4)cc3)c3cccc4c3oc3ccccc34)cc2)cc1]

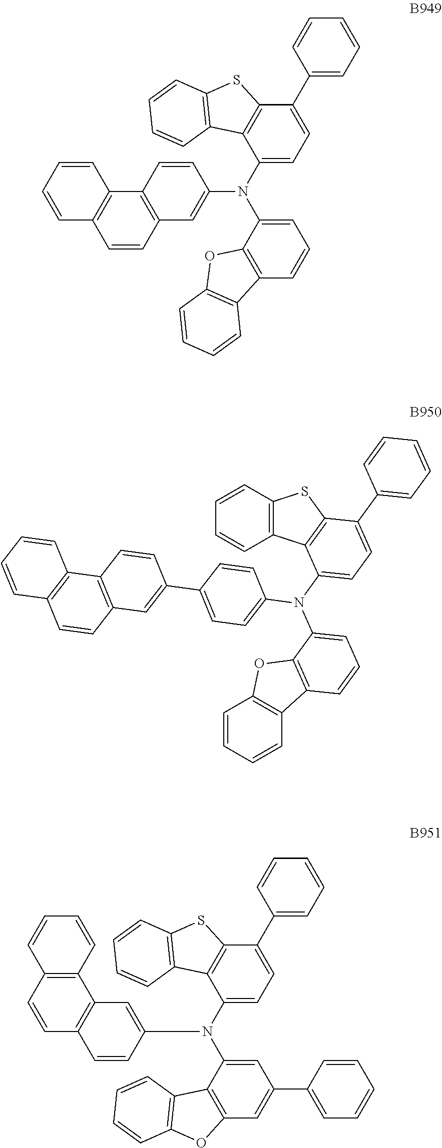 [SMILES: c1ccc(-c2ccc(N(c3ccc4c(ccc5ccccc54)c3)c3cccc4c3oc3ccccc34)c3c2sc2ccccc23)cc1 ; c1ccc(-c2ccc(N(c3ccc(-c4ccc5c(ccc6ccccc65)c4)cc3)c3cccc4c3oc3ccccc34)c3c2sc2ccccc23)cc1 ; c1ccc(-c2cc(N(c3ccc4ccc5ccccc5c4c3)c3ccc(-c4ccccc4)c4sc5ccccc5c34)c3c(c2)oc2ccccc23)cc1]

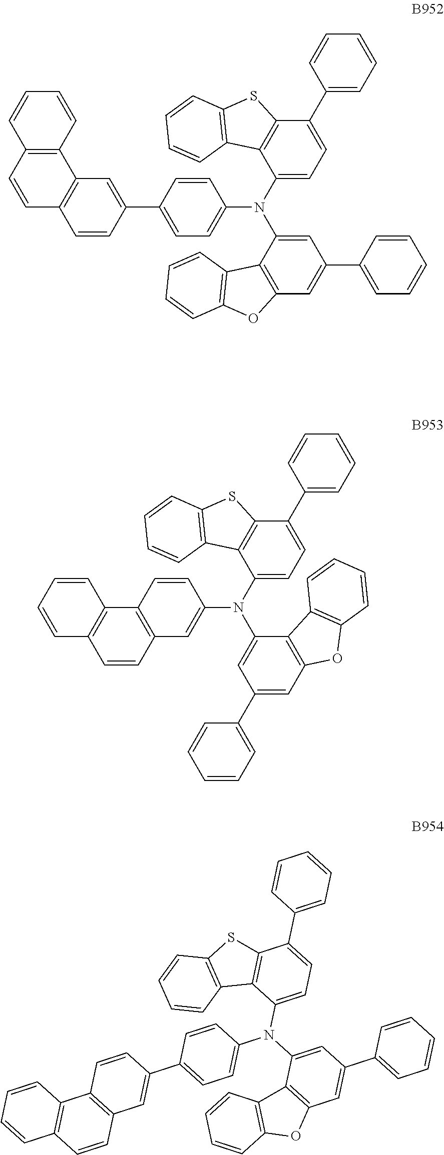 [SMILES: c1ccc(-c2cc(N(c3ccc(-c4ccc5ccc6ccccc6c5c4)cc3)c3ccc(-c4ccccc4)c4sc5ccccc5c34)c3c(c2)oc2ccccc23)cc1 ; c1ccc(-c2cc(N(c3ccc4c(ccc5ccccc54)c3)c3ccc(-c4ccccc4)c4sc5ccccc5c34)c3c(c2)oc2ccccc23)cc1 ; c1ccc(-c2cc(N(c3ccc(-c4ccc5c(ccc6ccccc65)c4)cc3)c3ccc(-c4ccccc4)c4sc5ccccc5c34)c3c(c2)oc2ccccc23)cc1]

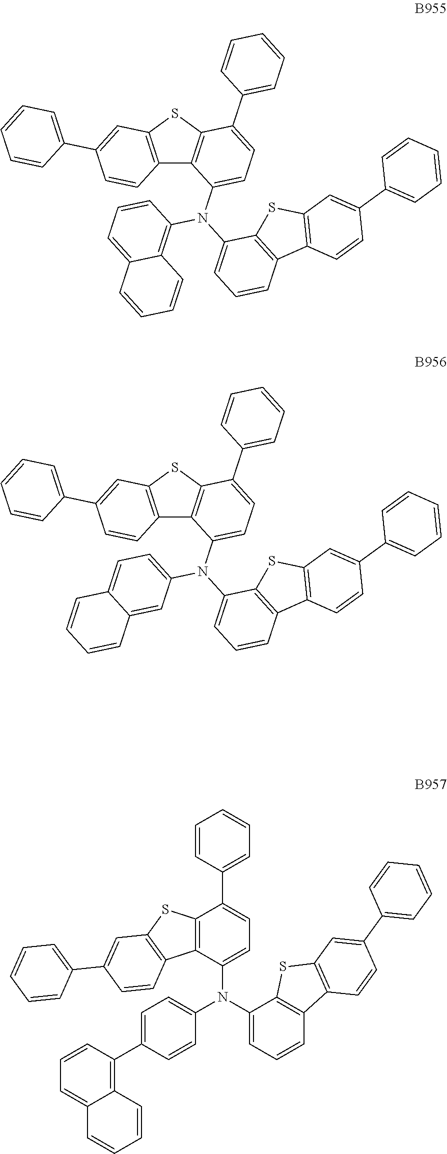 [SMILES: c1ccc(-c2ccc3c(c2)sc2c(N(c4cccc5ccccc45)c4ccc(-c5ccccc5)c5sc6cc(-c7ccccc7)ccc6c45)cccc23)cc1 ; c1ccc(-c2ccc3c(c2)sc2c(N(c4ccc5ccccc5c4)c4ccc(-c5ccccc5)c5sc6cc(-c7ccccc7)ccc6c45)cccc23)cc1 ; c1ccc(-c2ccc3c(c2)sc2c(N(c4ccc(-c5cccc6ccccc56)cc4)c4ccc(-c5ccccc5)c5sc6cc(-c7ccccc7)ccc6c45)cccc23)cc1]

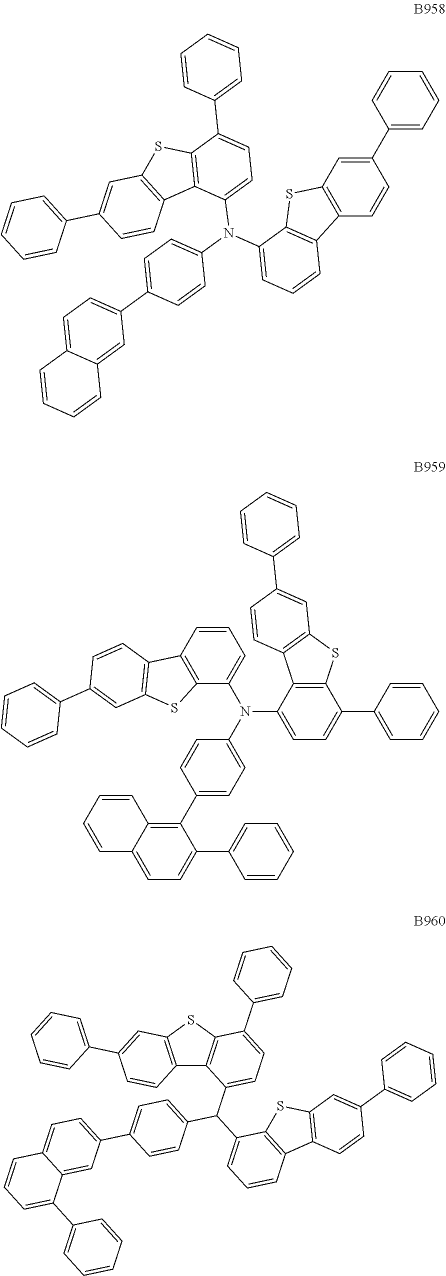 [SMILES: c1ccc(-c2ccc3c(c2)sc2c(N(c4ccc(-c5ccc6ccccc6c5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5c(-c6ccccc6)ccc6ccccc56)cc4)c4ccc(-c5ccccc5)c5sc6cc(-c7ccccc7)ccc6c45)cccc23)cc1 ; c1ccc(-c2ccc3c(c2)sc2c(C(c4ccc(-c5ccc6cccc(-c7ccccc7)c6c5)cc4)c4ccc(-c5ccccc5)c5sc6cc(-c7ccccc7)ccc6c45)cccc23)cc1]

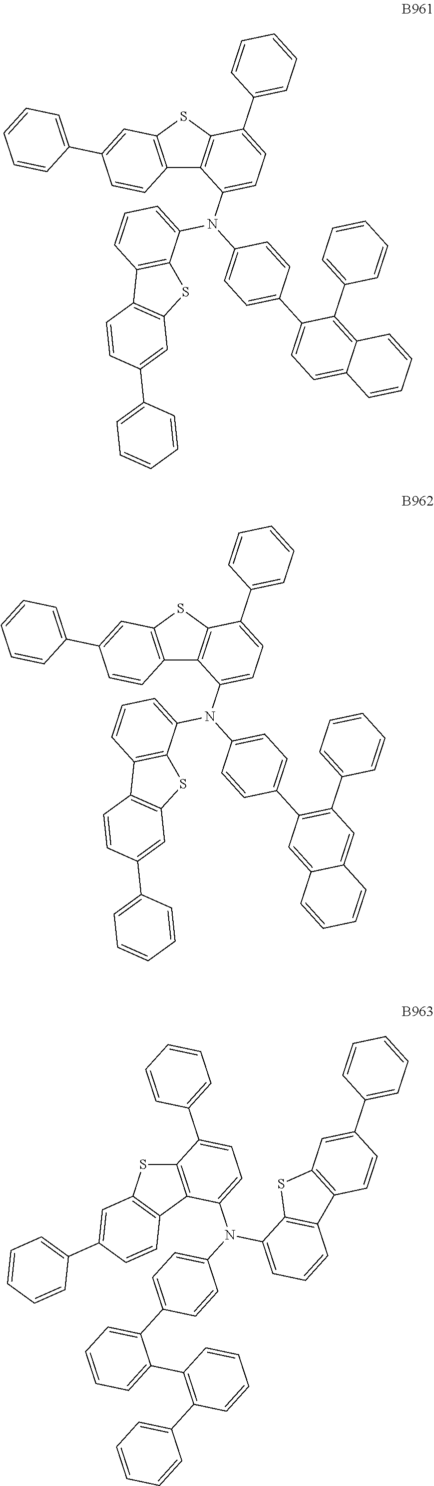 [SMILES: c1ccc(-c2ccc3c(c2)sc2c(N(c4ccc(-c5ccc6ccccc6c5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5cc6ccccc6cc5-c5ccccc5)cc4)c4ccc(-c5ccccc5)c5sc6cc(-c7ccccc7)ccc6c45)cccc23)cc1 ; c1ccc(-c2ccc3c(c2)sc2c(N(c4ccc(-c5ccccc5-c5ccccc5-c5ccccc5)cc4)c4ccc(-c5ccccc5)c5sc6cc(-c7ccccc7)ccc6c45)cccc23)cc1]

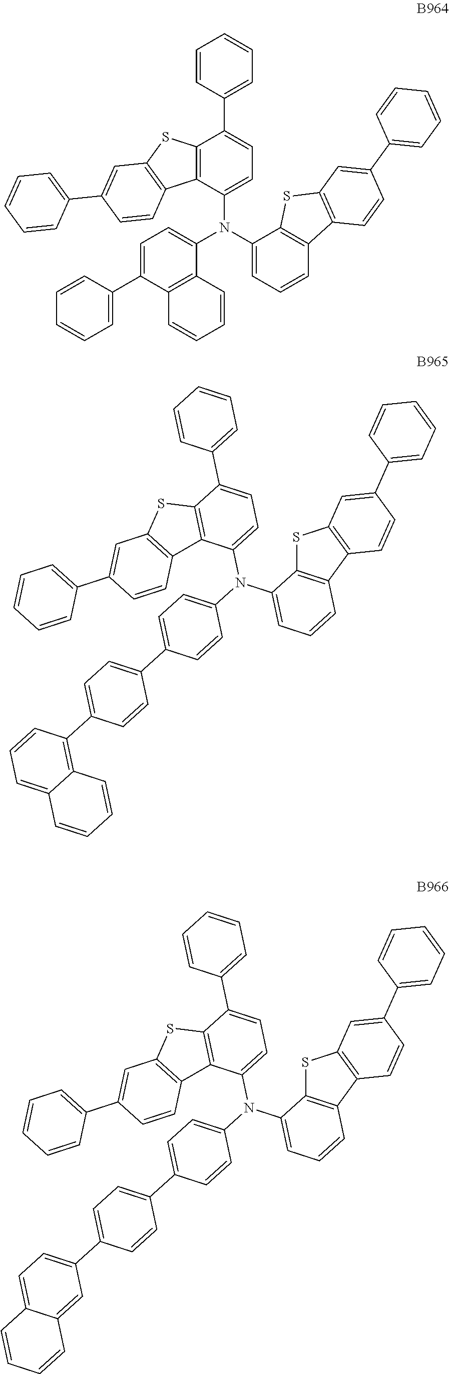

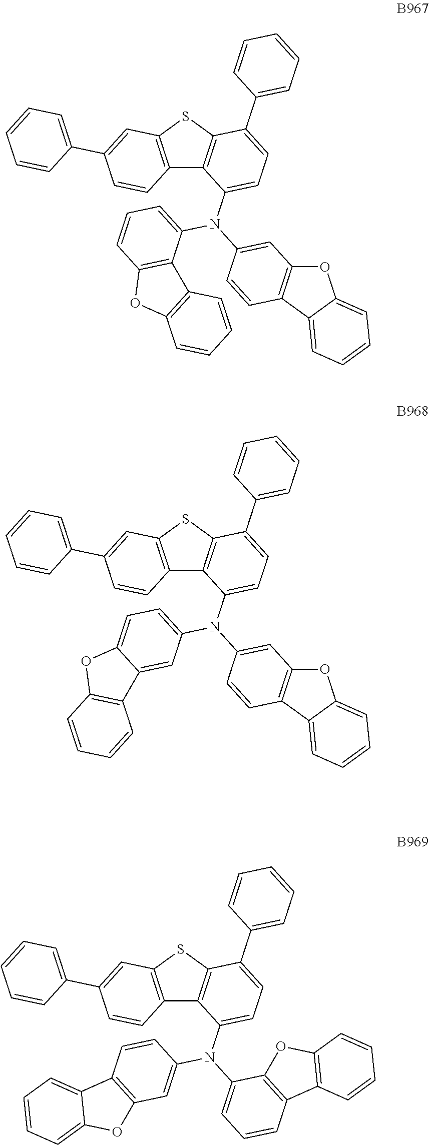 [SMILES: c1ccc(-c2ccc3c(c2)sc2c(-c4ccccc4)ccc(N(c4ccc5c(c4)oc4ccccc45)c4cccc5oc6ccccc6c45)c23)cc1 ; c1ccc(-c2ccc3c(c2)sc2c(-c4ccccc4)ccc(N(c4ccc5c(c4)oc4ccccc45)c4ccc5oc6ccccc6c5c4)c23)cc1 ; c1ccc(-c2ccc3c(c2)sc2c(-c4ccccc4)ccc(N(c4ccc5c(c4)oc4ccccc45)c4cccc5c4oc4ccccc45)c23)cc1]

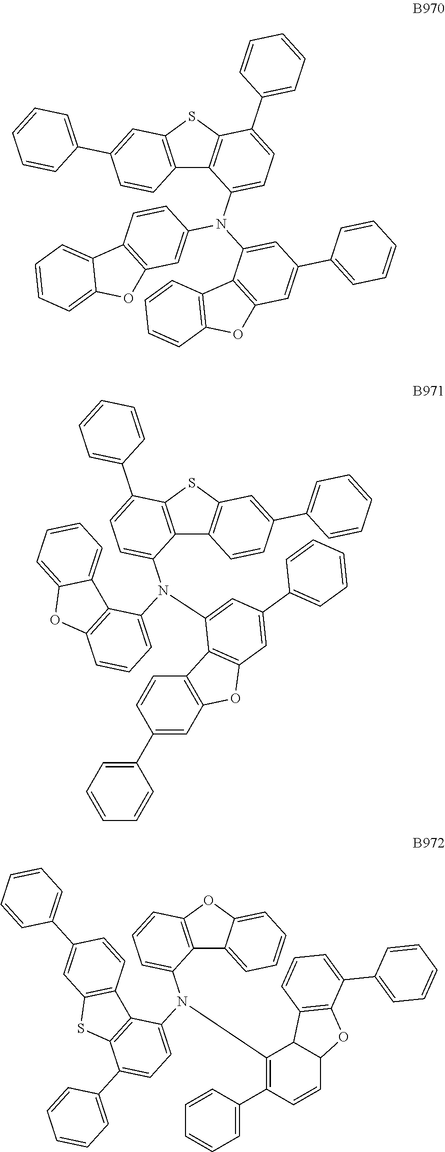 [SMILES: c1ccc(-c2cc(N(c3ccc4c(c3)oc3ccccc34)c3ccc(-c4ccccc4)c4sc5cc(-c6ccccc6)ccc5c34)c3c(c2)oc2ccccc23)cc1 ; c1ccc(-c2ccc3c(c2)oc2cc(-c4ccccc4)cc(N(c4cccc5oc6ccccc6c45)c4ccc(-c5ccccc5)c5sc6cc(-c7ccccc7)ccc6c45)c23)cc1 ; C1=CC2Oc3c(-c4ccccc4)cccc3C2C(N(c2cccc3oc4ccccc4c23)c2ccc(-c3ccccc3)c3sc4cc(-c5ccccc5)ccc4c23)=C1c1ccccc1]

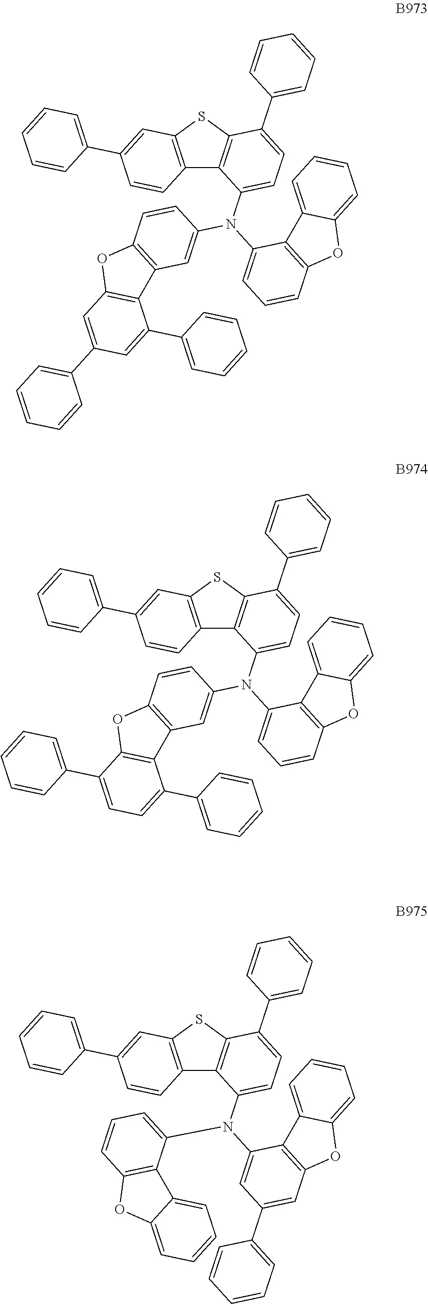 [SMILES: c1ccc(-c2cc(-c3ccccc3)c3c(c2)oc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)ccc(-c7ccccc7)c6c5c4)c4cccc5oc6ccccc6c45)c23)cc1 ; c1ccc(-c2cc(N(c3cccc4oc5ccccc5c34)c3ccc(-c4ccccc4)c4sc5cc(-c6ccccc6)ccc5c34)c3c(c2)oc2ccccc23)cc1]

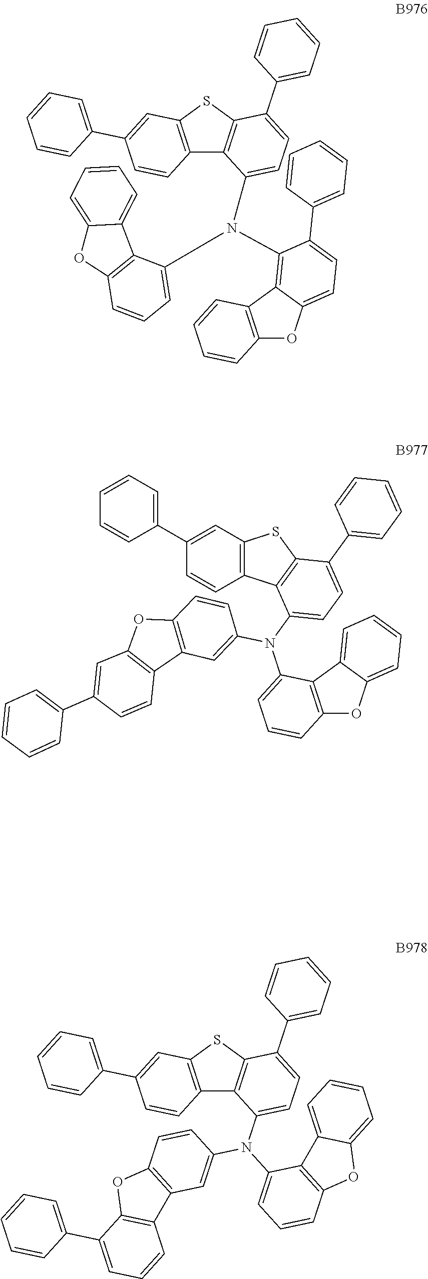 [SMILES: c1ccc(-c2ccc3c(c2)sc2c(-c4ccccc4)ccc(N(c4cccc5oc6ccccc6c45)c4c(-c5ccccc5)ccc5oc6ccccc6c45)c23)cc1 ; c1ccc(-c2ccc3c(c2)oc2ccc(N(c4cccc5oc6ccccc6c45)c4ccc(-c5ccccc5)c5sc6cc(-c7ccccc7)ccc6c45)cc23)cc1 ; c1ccc(-c2ccc3c(c2)sc2c(-c4ccccc4)ccc(N(c4ccc5oc6c(-c7ccccc7)cccc6c5c4)c4cccc5oc6ccccc6c45)c23)cc1]

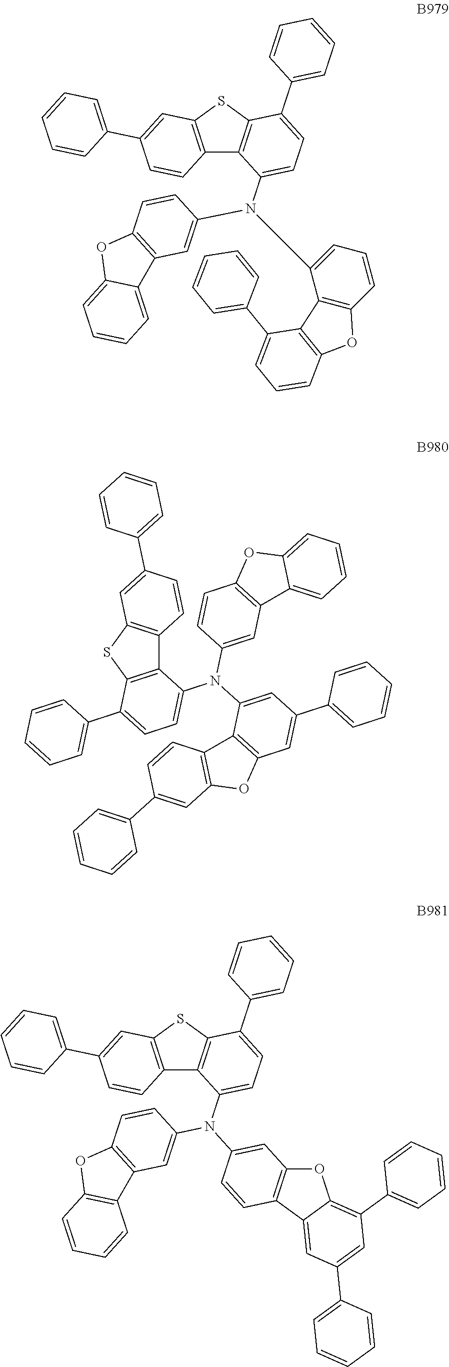 [SMILES: c1ccc(-c2ccc3c(c2)sc2c(-c4ccccc4)ccc(N(c4ccc5oc6ccccc6c5c4)c4cccc5oc6cccc(-c7ccccc7)c6c45)c23)cc1 ; c1ccc(-c2ccc3c(c2)oc2cc(-c4ccccc4)cc(N(c4ccc5oc6ccccc6c5c4)c4ccc(-c5ccccc5)c5sc6cc(-c7ccccc7)ccc6c45)c23)cc1 ; c1ccc(-c2ccc3c(c2)sc2c(-c4ccccc4)ccc(N(c4ccc5c(c4)oc4c(-c6ccccc6)cc(-c6ccccc6)cc45)c4ccc5oc6ccccc6c5c4)c23)cc1]

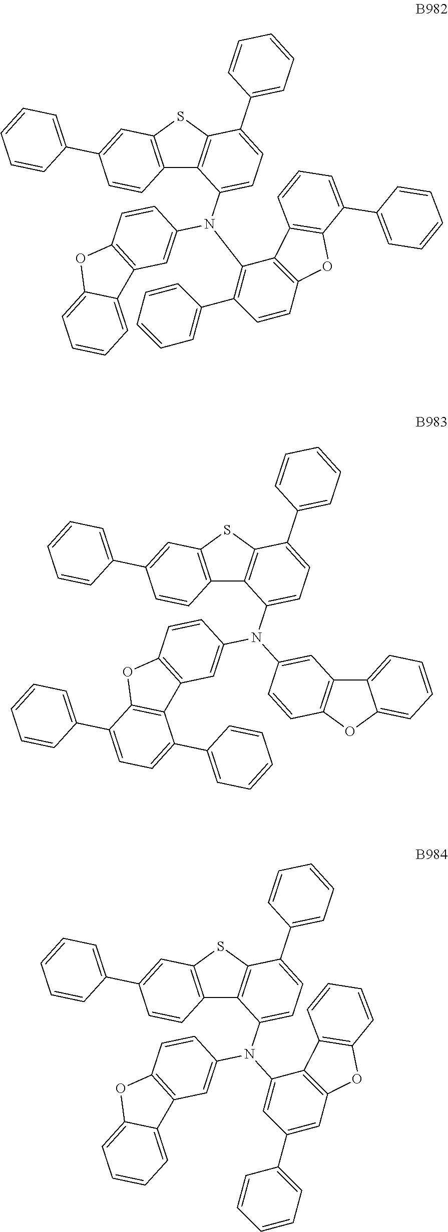 [SMILES: c1ccc(-c2ccc3c(c2)sc2c(-c4ccccc4)ccc(N(c4ccc5oc6ccccc6c5c4)c4c(-c5ccccc5)ccc5oc6c(-c7ccccc7)cccc6c45)c23)cc1 ; c1ccc(-c2ccc3c(c2)sc2c(-c4ccccc4)ccc(N(c4ccc5oc6ccccc6c5c4)c4ccc5oc6c(-c7ccccc7)ccc(-c7ccccc7)c6c5c4)c23)cc1 ; c1ccc(-c2cc(N(c3ccc4oc5ccccc5c4c3)c3ccc(-c4ccccc4)c4sc5cc(-c6ccccc6)ccc5c34)c3c(c2)oc2ccccc23)cc1]

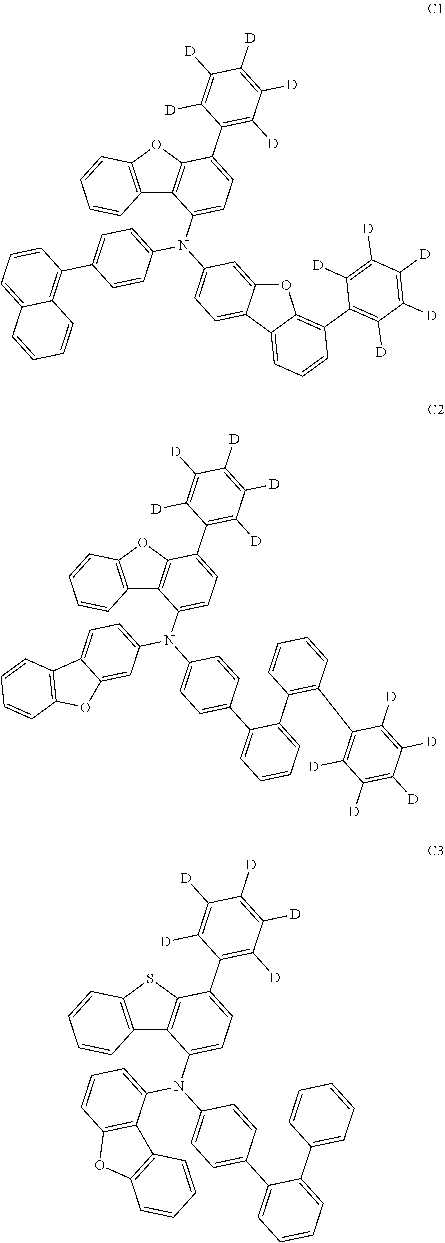 [SMILES: [2H]c1c([2H])c([2H])c(-c2cccc3c2oc2cc(N(c4ccc(-c5cccc6ccccc56)cc4)c4ccc(-c5c([2H])c([2H])c([2H])c([2H])c5[2H])c5oc6ccccc6c45)ccc23)c([2H])c1[2H] ; [2H]c1c([2H])c([2H])c(-c2ccccc2-c2ccccc2-c2ccc(N(c3ccc4c(c3)oc3ccccc34)c3ccc(-c4c([2H])c([2H])c([2H])c([2H])c4[2H])c4oc5ccccc5c34)cc2)c([2H])c1[2H] ; [2H]c1c([2H])c([2H])c(-c2ccc(N(c3ccc(-c4ccccc4-c4ccccc4)cc3)c3cccc4oc5ccccc5c34)c3c2sc2ccccc23)c([2H])c1[2H]]

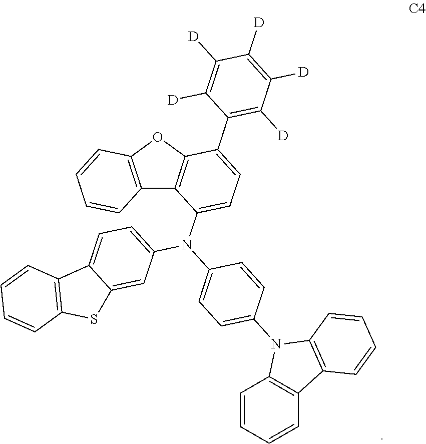 [SMILES: [2H]c1c([2H])c([2H])c(-c2ccc(N(c3ccc(-n4c5ccccc5c5ccccc54)cc3)c3ccc4c(c3)sc3ccccc34)c3c2oc2ccccc23)c([2H])c1[2H]]

23. A display device, comprising:
a base layer;
a circuit layer on the base substrate; and
a display element layer on the circuit layer and including a light emitting element;
wherein the light emitting element comprises
a first electrode;
a second electrode on the first electrode; and
at least one functional layer between the first electrode and the second electrode, and comprising an amine compound represented by the following Formula 1: 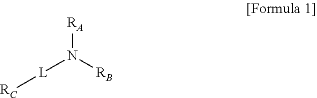
in Formula 1,
R_{A} is represented by Formula 2,
R_{B} is represented by Formula 3,
R_{C} is a substituted or unsubstituted aryl group of 6 to 30 ring-forming carbon atoms, or a substituted or unsubstituted heteroaryl group of 2 to 30 ring-forming carbon atoms,
L is a direct linkage, or a substituted or unsubstituted arylene group of 6 to 30 ring-forming carbon atoms,
R_{C} is not a substituted or unsubstituted benzonaphthofuran group, a substituted or unsubstituted benzonaphthothiophene group, or a substituted or unsubstituted benzofluorene group,
when R_{C} is a substituted or unsubstituted dibenzofuran group, or a substituted or unsubstituted dibenzothiophene group, L is a direct linkage, and
when R_{C} is a substituted or unsubstituted carbazole group, L is a direct linkage, or a substituted or unsubstituted phenylene group: 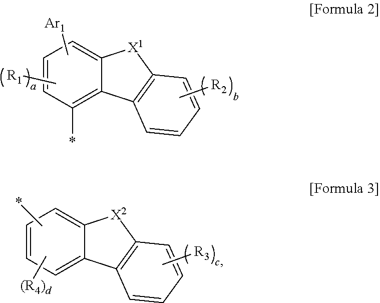
and
in Formula 2 and Formula 3,
Ar₁ is a substituted or unsubstituted aryl group of 6 to 30 ring-forming carbon atoms,
X₁ and X₂ are each independently O or S,
R₁ to R₄ are each independently a hydrogen atom, a deuterium atom, a halogen atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms,
wherein,
R₁ to R₄ are not each a substituted or unsubstituted nitrogen-containing six-member heterocycle,
Ar₁ is not a substituted or unsubstituted dihydronaphthyl group, or a substituted or unsubstituted p-aminophenyl group,
R₃ is not a substituted or unsubstituted fluorene group,
a is an integer of 0 to 2,
d is an integer of 0 to 3,
b and c are each independently an integer of 0 to 4, and
in Formula 2 when X₁ is O, Formula 3 is not represented by Formula 3-a:

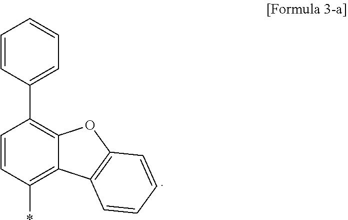 [SMILES: *c1ccc(-c2ccccc2)c2oc3ccccc3c12]

24. The display device of claim 23, wherein the light emitting element further comprises a capping layer, and
wherein the refractive index of the capping layer in a wavelength range of about 550 nm to about 660 nm is about 1.6 or more.

25. The display device of claim 23, further comprising:
a light controlling layer dispose on the display element layer and having a quantum dot,
wherein the light emitting element emits a first color light, and
wherein the light controlling layer comprises:
a first light controlling part comprising a first quantum dot which converts the first color light into second color light in a longer wavelength region than the first color light;
a second light controlling part comprising a second quantum dot which converts the first color light into third color light in a longer wavelength region than each of the first color light and the second color light; and
a third light controlling part which transmits the first color light therethrough.

26. The display device of claim 25, further comprising:
a color filter layer disposed on the light controlling layer,
wherein the color filter layer comprises:
a first filter which transmits the second color light;
a second filter which transmits the third color light; and
a third filter which transmits the first color light.
